(19) United States
(12) Patent Application Publication
(10) Pub. No.: US 2024/0285956 A1
(43) Pub. Date: Aug. 29, 2024

(54) METHODS AND DEVICES FOR SECURE COMMUNICATION WITH AND OPERATION OF AN IMPLANT

(71) Applicant: Peter Forsell, Bouveret (CH)

(72) Inventor: Peter Forsell, Bouveret (CH)

(21) Appl. No.: 18/443,345

(22) Filed: Feb. 16, 2024

Related U.S. Application Data
(63) Continuation-in-part of application No. PCT/EP2022/073863, filed on Aug. 26, 2022.
(63) Continuation-in-part of application No. PCT/EP2023/053987, filed on Feb. 16, 2023.

(30) Foreign Application Priority Data
Aug. 30, 2021 (WO) PCT/EP2021/073893
Feb. 18, 2022 (SE) 2250226-4

(51) Int. Cl. A61N 1/372; A61N 1/378; H04B 1/38

(52) U.S. Cl. CPC A61N 1/37254; A61N 1/37223; A61N 1/3787; H04B 1/38

(57) ABSTRACT

The present disclosure relates to an apparatus for powering an implant for a human patient and a method for powering an implant for a human patient. Wherein said apparatus comprises an implantable energy source for providing energy to the implant, an energy provider connected to the implantable energy source and connected to an energy consuming part of the implant.

This application is a continuation in part of International Application No. PCT/EP2023/053987, filed Aug. 26, 2022, and a continuation in part of International Application No. PCT/EP2022/073863, filed Aug. 26, 2022, which designates the US and claims priority to Swedish Application No 2250226-4, filed Feb. 18, 2022 and to International Application No. PCT/EP2021/073893 filed Aug. 30, 2021, the entire contents of each of which are hereby incorporated by reference.

### TECHNICAL FIELD

This disclosure generally relates to an implant, and in particular to methods and devices which facilitate secure communication with and operation of the implant.

### BACKGROUND

A medical implant is designed to be subcutaneously implanted in a patient's body. The new generation of implants is getting more advanced, and some implants may obtain, read and/or store data. This data can consist of various information, relating to for example different physiological parameters of the patient's body. For some implants, this data can be transferred via wires or wirelessly communicated to other external or internal devices. Some data may contain sensitive information and therefore require a reliable communication approach in order to avoid unauthorized recipients to gain access to it. Other sensitive data being transferred to and/or from the implant may contain information on program updates or control data for programs controlling the implant. Such data must also be protected to ensure that the implant is only controlled by authorized users.

There is thus a need for improvements within this context.

### SUMMARY

In view of the above, it is thus an object of the present invention to overcome or at least mitigate the problems discussed above.

An external device configured for communication with the implantable medical device according to any of the embodiments herein, when implanted in a patient, is further provided. The external device comprises at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol.

According to one embodiment, the second wireless transceiver comprises a Bluetooth transceiver.

According to one embodiment, the external device is further configured to communicate with a second external device using the at least one wireless transceiver.

According to one embodiment, the external device is configured for determining a distance between the external device and the implantable medical device by determining the RSSI.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the first network protocol is less than a communication range of the second network protocol.

According to one embodiment, a frequency band of the first network protocol differs from a frequency band of the second network protocol.

According to one embodiment, the external device is configured to authenticate the implantable medical device if the determined distance between the external device and the implantable medical device is less than a predetermined threshold value. The external device may be configured to allow the transfer of data between the external device and the implantable medical device after the implantable medical device has been authenticated.

According to one embodiment, the external device is a wearable external device.

According to one embodiment, the external device is a handset.

An implantable medical device configured for communication with an external device according to one of the embodiments herein is further provided. The implantable medical device comprising at least one first wireless transceiver configured for communication with the external device using a first network protocol, for determining a distance between the external device and the implantable medical device, and at least one second wireless transceiver configured for communication with the external device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol.

According to one embodiment, the second wireless transceiver comprises a Bluetooth transceiver.

According to one embodiment, the implantable medical device is further configured to communicate with a second external device using said at least one wireless transceiver.

According to one embodiment, the implantable medical device is configured for determining a distance between the external device and the implantable medical device by determining the RSSI.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the first network protocol may be less than the communication range of the second network protocol.

The frequency band of the first network protocol may differ from a frequency band of the second network protocol.

According to one embodiment, the implantable medical device is configured to authenticate the external device if the determined distance between the external device and the implantable medical device is less than a predetermined threshold value.

According to one embodiment, the implantable medical device may be configured to allow the transfer of data between the implantable medical device and the external device after the external device has been authenticated.

An external device configured for communication with an implantable medical device according to any one of the embodiments disclosed herein is further provided. The external device comprising a wireless communication unit configured for wireless transmission of control commands to the implantable medical device and configured for wireless communication with a display device, and a computing unit configured for running a control software for creating the control commands for the operation of the implantable medical device. The computing unit may be configured to transmit a control interface to a display device configured to display the control interface to a user, receive user input from the display device, and transform the user input into the control commands for wireless transmission to the implantable medical device.

In one embodiment, the wireless communication unit comprises a wireless transceiver for wireless transmission of control commands to the implantable medical device, and wireless transmission of the control interface to the display device.

According to one embodiment, the wireless communication unit comprises a first wireless transceiver for wireless transmission of control commands to the implantable medical device, and a second wireless transceiver for wireless transmission of the control interface to the display device.

The wireless communication unit may in one embodiment be configured for wireless communication with the display device using a standard network protocol.

In one embodiment, the wireless communication unit is configured for wireless communication with the implantable medical device using a proprietary network protocol.

The wireless communication unit may comprise a Bluetooth transceiver, which may be comprised in one of the first and second wireless transceiver.

According to one embodiment, the wireless communication unit comprises a UWB transceiver, which may be comprised in one of the first and second wireless transceiver.

The wireless communication unit may comprise at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver is configured for transcutaneous energy transfer for at least one of: powering an energy consuming component of the implantable medical device, and charging an implantable energy storage unit.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the first wireless transceiver may be less than a communication range of the second wireless transceiver

The frequency band of the first network protocol may differ from a frequency band of the second network protocol.

According to one embodiment, the external device is configured to authenticate the implantable medical device if a distance between the external device and the implantable medical device is less than a predetermined threshold value.

According to one embodiment, the external device is configured to be authenticated by the implantable medical device if a distance between the external device and the implantable medical device is less than a predetermined threshold value.

According to one embodiment, the external device is configured to authenticate the display device if a distance between the external device and the display device is less than a predetermined threshold value.

According to one embodiment, the external device is configured to be authenticated by the implantable medical device if a distance between the external device and the display device is less than a predetermined threshold value.

The external device may be configured to allow the transfer of data between the external device and the implantable medical device, and/or the external device and the display device, on the basis of the authentication.

According to one embodiment, the computing unit is configured to encrypt at least one of the control interface and the control commands.

A display device for communication with an external device for communication with an implantable medical device is further provided. The display device comprises a wireless communication unit configured for wirelessly receiving an implant control interface from the external device and configured for wirelessly transmitting implant control user input to the external device. The display device further comprising a display for displaying the received implant control interface, and an input device for receiving implant control input from the user.

According to one embodiment, the display device further comprises an auxiliary wireless communication unit configured to be disabled to enable at least one of: wirelessly receiving the implant control interface from the external device, and wirelessly transmitting implant control user input to the external device.

According to one embodiment, the wireless communication unit is configured for wireless communication with the external device using a standard network protocol.

According to one embodiment, the wireless communication unit is configured for wireless communication with the external device using a proprietary network protocol.

According to one embodiment, the wireless communication unit comprises a Bluetooth transceiver.

According to one embodiment, the wireless communication unit comprises a UWB transceiver.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

The communication range of the wireless communication unit of the display device may be less than a communication range of the auxiliary wireless communication unit.

According to one embodiment, the display device is configured to authenticate the external device if a distance between the display device and the external device is less than a predetermined threshold value.

According to one embodiment, the display device is configured to be authenticated by the external device if a distance between the display device and the external device is less than a predetermined threshold value.

According to one embodiment, the display device is configured to allow the transfer of data between the display device and the external device on the basis of the authentication.

The display device may be a wearable external device or a handset.

A communication system for enabling communication between a display device and an implantable medical device is further provided. The communication system comprising a display device, a server, and an external device. The display device comprises a wireless communication unit configured for wirelessly receiving an implant control interface from the server, the implant control interface being provided by the external device, the wireless communication unit further being configured for wirelessly transmitting implant control user input to the server, destined for the external device, a display for displaying the received implant control interface, and an input device for receiving implant control input from the user. The server of the communication system comprises: a wireless communication unit configured for wirelessly receiving an implant control interface from the external device and wirelessly transmitting the implant control interface to the display device, the wireless communication unit further being configured for wirelessly receiving implant control user input from the display device and wirelessly transmitting the implant control user input to the external device. The external device of the communication system comprises a wireless communication unit configured for wireless transmission of control commands to the implantable medical device and configured for wireless communication with the server, and a computing unit configured for: running a control software for creating the control
commands for the operation of the implantable medical device, transmit a control interface to the server, destined for the display device, receive implant control user input generated at the
display device, from the server, and transform the user input into the control commands for wireless transmission to the implantable medical device.

According to one embodiment, the computing unit of the communication system is configured to encrypt at least one of the control interface and the control commands.

According to one embodiment, the display device is configured to encrypt the user input.

According to one embodiment, the server is configured to encrypt at least one of the user input received from the display device and the control interface received from the external device.

According to one embodiment, the computing unit is configured to encrypt the control interface and the display device is configured to decrypt the encrypted control interface.

According to one embodiment, the server is configured to act as a router, transferring the encrypted control interface from the external device to the display device without decryption.

A display device for communication with an external device for communication with an implantable medical device is further provided. The display device comprising a wireless communication unit, a display, and an input device for receiving implant control input from the user. The display device is configured to run a first application for wireless communication with a server, and to run a second application for wireless communication with the external device for transmission of the implant control input to the external device for the communication with the implantable medical device, wherein the second application is configured to be accessed through the first application. The display device may comprise a first log-in function and a second log-in function, and wherein the first log-in function gives the user access to the first application and wherein the first and second log-in function in combination gives the user access to the second application.

According to one embodiment, the first log-in is a PIN-based log-in.

According to one embodiment, at least one of the first and second log-in is a log-in based on a biometric input or a hardware key.

According to one embodiment, the display device further comprises an auxiliary wireless communication unit, and the auxiliary wireless communication unit is configured to be disabled to enable wireless communication with the external device.

According to one embodiment, the display device is configured to wirelessly receive an implant control interface from the external device to be displayed on the display.

According to one embodiment of the display device, the wireless communication unit is configured for wireless communication with the external device using a standard network protocol.

According to one embodiment of the display device, the wireless communication unit is configured for wireless communication with the external device using a proprietary network protocol.

According to one embodiment of the display device, the wireless communication unit is configured for wireless communication with the external device using a first network protocol and with the server using a second network protocol.

According to one embodiment of the display device, the wireless communication unit is configured for wireless communication with the external device using a first frequency band and with the server using a second frequency band.

According to one embodiment of the display device, the wireless communication unit comprises a Bluetooth transceiver.

According to one embodiment of the display device, the wireless communication unit comprises a UWB transceiver.

According to one embodiment, the standard network protocol is one of, or a combination of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the communication range of the wireless communication unit is less than a communication range of the auxiliary wireless communication unit.

According to one embodiment, the wireless communication unit comprises a first wireless transceiver for communication with the external device and a second wireless transceiver for communication with the server.

The second wireless transceiver may be configured to be disabled to enable wireless communication using the first wireless transceiver.

According to one embodiment, the display device is configured to authenticate the external device if a distance between the display device and the external device is less than a predetermined threshold value, and the display device is configured to be authenticated by the external device if a distance between the display device and the external device is less than a predetermined threshold value.

According to one embodiment, the display device is configured to allow the transfer of data between the display device and the external device on the basis of the authentication.

The display device may be a wearable external device or a handset.

According to one embodiment of the display device, the second application may be configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, or an error.

According to one embodiment, the display device is configured to encrypt the user input.

According to one embodiment, the display is configured to encrypt the user input for decryption by the implantable medical device.

According to one embodiment, the display device is configured to decrypt the control interface received from the external device, for displaying the control interface on the display.

According to one embodiment, at least one of the first and second application is configured to receive data from an auxiliary external device and present the received data to the user.

At least one of the first and second application may be configured to receive data from an auxiliary external device comprising a scale for determining the weight of the user.

According to one embodiment, at least one of the first and second application may be configured to receive data related to the weight of the user from an auxiliary external device comprising a scale.

According to one embodiment, the display device is configured to: wirelessly transmit the data related to the weight of the user to the external device, or wirelessly transmit an instruction derived from the data related to the weight of the user, or wirelessly transmit an instruction derived from a combination of the data related to the weight of the user and the implant control input received from the user.

A communication system for enabling communication between a display device and an implantable medical device is further provided. The communication system comprises a display device, a server, and an external device. The display device comprises: a wireless communication unit configured for wirelessly receiving an implant control interface from the external device, the wireless communication unit further being configured for wirelessly transmitting implant control user input to the external device. The display device further comprises a display for displaying the received implant control interface, and an input device for receiving implant control input from the user, wherein the display device is configured to run a first application for wireless communication with the server, and to run a second application for wireless communication with the external device for transmission of the implant control input to the external device for the communication with the implantable medical device. The external device comprises a wireless communication unit configured for wireless transmission of control commands based on the implant control input to the implantable medical device and configured for wireless communication with the display device.

According to one embodiment, the display device comprises a first log-in function and a second log-in function, and wherein the first log-in function gives the user access to the first application and wherein the first and second log-in function in combination gives the user access to the second application.

The second application may be configured to receive data related to a parameter of the implanted medical device, and the second application may be configured to receive data related to a sensor value received from the implanted medical device.

According to one embodiment, the second application is configured to receive data related to a parameter related to at least one of: a battery status, a temperature, a time, or an error.

According to one embodiment, the display device is configured to encrypt the user input.

According to one embodiment, the display is configured to encrypt the user input for decryption by the implantable medical device.

According to one embodiment, the external device is configured to act as a router, transferring the encrypted user input from the display device to the implantable medical device without decryption.

According to one embodiment, the external device is configured to encrypt at least one of the control interface and the control commands.

According to one embodiment, the external device is configured to encrypt the control interface and wherein the display device is configured to decrypt the encrypted control interface.

A computer program product configured to run in a display device comprising a wireless communication unit, a display for displaying the received implant control interface, and an input device for receiving implant control input from a user is further provided. The computer program product comprising a first application for communication with a server, and a second application for communication with an external device for transmission of the implant control input to the external device for the communication with an implantable medical device, wherein the second application is configured to be accessed through the first application. The computer program product further comprises a first log-in function, and a second log-in function, wherein the first log-in function gives the user access to the first application and the first and second log-in function in combination gives the user access to the second application.

According to one embodiment of the computer program product, the second application is configured to receive data related to a parameter of the implanted medical device.

According to one embodiment of the computer program product, the second application is configured to receive data related to a sensor value received from the implanted medical device.

According to one embodiment of the computer program product, the second application is configured to receive data related to a parameter related to at least one of: a battery status, a temperature, a time, or an error.

A communication system for enabling communication between a display device, an external device, a server and an implantable medical device is further provided. The communication system comprising: a server, a display device, an external device, and an implantable medical device. The display device comprises: a wireless communication unit for wirelessly communicating with at least one of the external device and the server, a display, and an input device for receiving input from the user. The external device comprises: a wireless communication unit configured for wireless transmission of control commands to the implantable medical device and configured for wireless communication with at least one of the display device and the server. The server comprises: a wireless communication unit configured for wireless communication with at least one of the display device and the external device.

The implantable medical device comprises: a wireless communication unit configured for wireless communication with the external device. The implantable medical device comprises an encryption unit which is configured to encrypt data destined for the server, transmit the data to the server via the external device, wherein the external device acts as a router transferring the data without full decryption, or the implantable medical device comprises an encryption unit and is configured to: encrypt data destined for the display device, transmit the data to the display device via the external device, wherein the external device acts as a router transferring the data without full decryption, or the server comprises an encryption unit and is configured to: encrypt data destined for the implantable medical device, transmit the data to the implantable medical device via the external device, wherein the external device acts as a router transferring the data without full decryption, or the server comprises an encryption unit and is configured to: encrypt data destined for the implantable medical device, transmit the data to the implantable medical device via the display device and the external device, wherein the display device and the external device acts as a router transferring the data without full decryption, or the display device comprises an encryption unit and is configured to: encrypt data destined for the implantable medical device, transmit the data to the implantable medical device via the external device, wherein the external device acts as a router transferring the data without full decryption, or the display device comprises an encryption unit and is configured to: encrypt data destined for the implantable medical device, transmit the data to the implantable medical device via the server and the external device, wherein the server and the external device acts as a router transferring the data without full decryption.

According to one embodiment, the display device is configured to wirelessly receive an implant control interface from the external device to be displayed on the display.

According to one embodiment of the communication system, at least two of: the wireless communication unit of the server, the wireless communication unit of the display device, the wireless communication unit of the external device, and the wireless communication unit of the implantable medical device—is configured for wireless communication using a standard network protocol.

According to one embodiment, the at least two of: the wireless communication unit of the server, the wireless communication unit of the display device, the wireless communication unit of the external device, and the wireless communication unit of the implantable medical device—is configured for wireless communication using a proprietary network protocol.

According to one embodiment, the wireless communication unit of the external device is configured to: use a first network protocol for communication with the implantable medical device and use a second network protocol for communication with the server, or use a first network protocol for communication with the implantable medical device and use a second network protocol for communication with the display device.

According to one embodiment, the wireless communication unit of the external device is configured to: use a first frequency band for communication with the implantable medical device and use a second frequency band for communication with the server, or use a first frequency band for communication with the implantable medical device and use a second frequency band for communication with the display device.

According to one embodiment, the wireless communication unit of the display device is configured to use a first network protocol for communication with the external device and use a second network protocol for communication with the server.

According to one embodiment, the wireless communication unit of the display device is configured to use a first frequency band for communication with the external device and use a second frequency band for communication with the server.

According to one embodiment, the wireless communication unit of the server is configured to use a first network protocol for communication with the external device and use a second network protocol for communication with the display device.

According to one embodiment, the wireless communication unit of the server is configured to use a first frequency band for communication with the external device and use a second frequency band for communication with the display device.

According to one embodiment, the wireless communication unit of at least one of the server, the display device, the external device, and the implantable medical device comprises a Bluetooth transceiver.

According to one embodiment, the wireless communication unit of at least one of the server, the display device, the external device, and the implantable medical device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and a GSM type protocol.

According to one embodiment, the wireless communication unit of the external device comprises a first wireless transceiver for wireless communication with the implantable medical device, and a second wireless transceiver for wireless communication with the server, and wherein the second wireless transceiver has a longer effective range than the first wireless transceiver.

According to one embodiment, the wireless communication unit of the external device comprises a first wireless transceiver for wireless communication with the implantable medical device, and a second wireless transceiver for wireless communication with the display device, and wherein the second wireless transceiver has a longer effective range than the first wireless transceiver.

According to one embodiment, the wireless communication unit of the display device comprises a first wireless transceiver for wireless communication with the external device, and a second wireless transceiver for wireless communication with the server, and wherein the second wireless transceiver has a longer effective range than the first wireless transceiver.

According to one embodiment, the second wireless transceiver has an effective range being one of: 2 times, 4 times, 8 times 20 times, 50 times or 100 times longer than the first wireless transceiver.

According to one embodiment, the second wireless transceiver is configured to be disabled to enable wireless communication using the first wireless transceiver.

According to one embodiment, of the communication system, at least one of:
the display device is configured to authenticate the external device if a distance between the display device and the external device is less than a predetermined threshold value,
the display device is configured to be authenticated by the external device if a distance between the display device and the external device is less than a predetermined threshold value,
the display device is configured to authenticate the implantable medical device if a distance between the display device and the implantable medical device is less than a predetermined threshold value,
the display device is configured to be authenticated by the implantable medical device if a distance between the display device and the implantable medical device is less than a predetermined threshold value,
the external device is configured to authenticate the display device if a distance between the external device and the display device is less than a predetermined threshold value,
the external device is configured to be authenticated by the display device if a distance between the external device and the display device is less than a predetermined threshold value,
the external device is configured to authenticate the implantable medical device if a distance between the external device and the implantable medical device is less than a predetermined threshold value, and
the external device is configured to be authenticated by the implantable medical device if a distance between the external device and the implantable medical device is less than a predetermined threshold value.

According to one embodiment of the communication system, the display device may be configured to allow the transfer of data between the display device and the external device on the basis of the authentication.

According to one embodiment of the communication system, the external device is configured to allow the transfer of data between the display device and the external device on the basis of the authentication.

According to one embodiment of the communication system, the external device is configured to allow the transfer of data between the external device and the implantable medical device on the basis of the authentication.

According to one embodiment of the communication system, the display device is a wearable external device or a handset.

According to one embodiment of the communication system, the data encrypted by the implantable medical device is related to at least one of: a battery status, a temperature, a time, or an error.

A server for use in the communication system according to any one of the embodiments above is further provided. claims 1-24.

A display device for use in the communication system according to any one of the embodiments above is further provided.

An external device for use in the communication system according to any one of the embodiments above is further provided.

An implantable medical device for use in the communication system according to any one of the embodiments above is further provided.

Any embodiment, part of embodiment, method, or part of method may be combined in any applicable way.

According to one embodiment, the body engaging portion is a constriction device configured to constrict a luminary organ of a patient, and the body engaging portion may comprise an implantable constriction device.

According to one embodiment, the implantable constriction device comprises an implantable constriction device for constricting a luminary organ of the patient. The luminary organ could be an intestine of the patient, such as a colon or rectum of the patient or a region of a stoma of the patient.

According to one embodiment, the luminary organ could be a blood vessel of the patient. The implantable constriction device may be a device for constricting the venous blood flow leading from an erectile tissue for promoting the engorgement of the erectile tissue, or may be a device configured to constrict a portion of a blood vessel having an aneurysm.

According to one embodiment, the luminary organ could be the vas deference of the patient.

According to one embodiment, the body engaging portion could comprise an implantable element for actively emptying the urinary bladder of the patient, and the implantable element for actively emptying the urinary bladder of the patient could be configured to empty the bladder of the patient by compressing the urinary bladder from the outside thereof.

The implantable medical device/implant described and disclosed herein could comprises at least one of the following implantable components and/or functions:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
   an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
   an implant configured for stimulating the ampulla vas deference and creating temporary constriction.

### Aspect 244SE Implantable Reset Switch—Implant Comprising a Reset Function—Embodiments of the Aspect 244SE of the Disclosure

In a first part of aspect 244SE, an implant is provided. The implant comprises an internal computing unit configured to control a function of said implant. The internal computing unit comprises an internal memory configured to store: a first control program for controlling said function, and a second, updatable, control program for controlling said function of said implant. The implant comprises an internal communication unit comprising said internal computing unit and configured to communicate with an external device. The internal computing unit is configured to receive updates to the second control program via said internal communication unit. The implant further comprises a reset function of, connected to, or transmitted to said internal computing unit, said reset function being configured to make said internal computing unit switch from running said second control program to running said first control program.

According to some embodiments of the first part of the aspect 244SE, the reset function is configured to make said internal computing unit delete said second control program from said internal memory.

According to some embodiments of the first part of the aspect 244SE, the internal communication unit comprises an internal wireless transceiver for communicating wirelessly with said external device.

According to some embodiments of the first part of the aspect 244SE, the internal communication unit is configured to be in electrical connection with said external device and communicate with said external device using a body of a patient, in which the implant is implanted, as a conductor.

According to some embodiments of the first part of the aspect 244SE, the reset function is configured to be operated by palpating a skin of a patient in which the implant is implanted.

According to some embodiments of the first part of the aspect 244SE, the reset function is configured to be operated by penetration of a skin of a patient in which the implant is implanted.

According to some embodiments of the first part of the aspect 244SE, the reset function is configured to be operated by magnetic force from outside a body of a patient in which the implant is implanted.

According to some embodiments of the first part of the aspect 244SE, the reset function is configured to be operated by said magnetic force being applied for a duration of time exceeding 2 seconds.

According to some embodiments of the first part of the aspect 244SE, the reset function is configured to be operated by said magnetic force being applied for a duration of time exceeding 5 seconds.

According to some embodiments of the first part of the aspect 244SE, the reset function is configured to be operated by said magnetic force being applied for a duration of time exceeding 10 seconds.

According to some embodiments of the first part of the aspect 244SE, the implant further comprising a feedback unit configured to provide feedback related to said internal computing unit switching from running said second control program to running said first control program.

According to some embodiments of the first part of the aspect 244SE, the feedback unit is configured to provide visual feedback.

According to some embodiments of the first part of the aspect 244SE, the feedback unit is configured to provide audible feedback.

According to some embodiments of the first part of the aspect 244SE, the feedback unit is configured to provide tactile feedback.

According to some embodiments of the first part of the aspect 244SE, the feedback unit is configured to provide feedback in the form of a wireless signal.

According to some embodiments of the first part of the aspect 244SE, the internal memory is configured to store a third control program for controlling said function of said implant, wherein said internal computing unit is configured to update the second control program to the third control program.

According to some embodiments of the first part of the aspect 244SE, the implant has a first power supply for running the first control program, and a second power supply, different from the first power supply, for running the second control program.

According to some embodiments of the first part of the aspect 244SE, the first power supply comprises a first internal energy storage, and wherein the second power supply comprises a second internal energy storage.

According to some embodiments of the first part of the aspect 244SE, the first power supply comprises a first energy receiver, and wherein the second power supply comprises a second energy receiver.

According to some embodiments of the first part of the aspect 244SE, the first energy receiver is configured to receive energy via a RFID pulse.

According to some embodiments of the first part of the aspect 244SE, the implant further comprising a feedback unit, configured to provide feedback related to said internal computing unit switching from running said second control program to running said first control program, wherein said feedback pertains to an amount of energy received via the RFID pulse.

In a second part of the aspect 244SE, a method for switching between a first and a second control program for controlling a function of an implant is provided.

The implant comprising: an internal computing unit configured to control a function of said implant, said internal computing unit comprises an internal memory configured to store: a first control program for controlling said function, and a second, updatable, control program for controlling said function of said implant.

The implant further comprising an internal communication unit comprising said internal computing unit and configured to communicate with an external device, wherein said internal computing unit is configured to receive updates to the second control program via said internal communication unit, and a reset function of, or connected to said internal computing unit, said reset function being configured to make said internal computing unit switch from running said second control program to running said first control program.

The method comprising the steps of activating said reset function, and instructing, by the reset function, said internal computing unit to switch from running said second control program to running said first control program.

According to some embodiments of the second part of the aspect 244SE, the method further comprising the step of: deleting, by the internal computing unit, said second control program from said internal memory.

According to some embodiments of the second part of the aspect 244SE, the internal memory is configured to store a third control program for controlling said function of said implant, wherein said internal computing unit is configured to update the second control program to the third control program, the method further comprising the steps of: updating the second control program to the third control program.

According to some embodiments of the second part of the aspect 244SE, the method further comprising the step of switching, by the internal computing unit, from running said first control program to running said first second program after updating the second control program.

According to some embodiments of the second part of the aspect 244SE, the internal communication unit is configured to be in electrical connection with said external device, and communicate with said external device using a body of a patient in which the implant is implanted as a conductor, the method further comprising the steps of: communicating, from said external device, to the internal communication unit, an update of the second control program, switching, by the internal computing unit, from running said first control program to running said first second program after updating the second control program.

According to some embodiments of the second part of the aspect 244SE, the step of activating said reset function comprises: palpating a skin of a patient in which the implant is implanted.

According to some embodiments of the second part of the aspect 244SE, wherein the step of activating said reset function comprises penetration of a skin of a patient in which the implant is implanted.

According to some embodiments of the second part of the aspect 244SE, the step of activating said reset function comprises applying a magnetic force from outside a body of a patient in which the implant is implanted.

According to some embodiments of the second part of the aspect 244SE, the method further comprising the step of providing feedback, by a feedback unit of the implant, said feedback related to said internal computing unit switching from running said second control program to running said first control program.

According to some embodiments of the second part of the aspect 244SE, the implant has a first power supply for running the first control program, and a second power supply, different from the first power supply, for running the second control program, wherein the first power supply comprises a first energy receiver, and wherein the second power supply comprises a second energy receiver, the method further comprising the steps of: providing, by an energy transmitter of the external device, energy to the first energy receiver.

According to some embodiments of the second part of the aspect 244SE, the step of providing, by the energy transmitter of the external device, energy to the first energy receiver comprises providing energy using a RFID pulse.

According to some embodiments of the second part of the aspect 244SE, the implant has a feedback unit, configured to provide feedback related to said internal computing unit, the method further comprising: providing, by the feedback unit, feedback to the said energy transmitter, wherein said feedback pertains to an amount of energy received via the RFID pulse, and adjusting, by the energy transmitter, a parameter of a subsequent RFID pulse based on the feedback.

According to some embodiments of the second part of the aspect 244SE, the parameter of the subsequent RFID pulse comprises at least one an energy level, a pulse frequency, and a pulse amplitude.

The above method according to the aspect 244SE may be implemented in software, which may be a computer program product comprising a computer-readable storage medium with instructions adapted to carry out the method when executed by a device having processing capability. The device having processing capacity being a computing unit in an external device or in the implant.

According to some embodiments of the first part of the aspect 244SE the reset function is a reset switch.

According to some embodiments of the second part of the aspect 244SE the reset function is a reset switch.

According to some embodiments of the first part of the aspect 244SE the internal computing unit is further configured for
receiving, from said external device, an update of the second control program,
updating the second control program,
switching, by the internal computing unit, from running said first control program to running said second program after updating the second control program.

According to some embodiments of the first part of the aspect 244SE the reset function is triggered by an update of the first or second control program.

According to some embodiments of the first part of the aspect 244SE the reset function is triggered by a malfunction of the first or second control program.

According to some embodiments of the first part of the aspect 244SE the reset function is triggered by a malfunction of an active device of the implant.

According to some embodiments of the first part of the aspect 244SE said reset function is configured to be operated by NFC.

According to some embodiments of the first part of the aspect 244SE the reset function is configured to trigger implant diagnostics to be transmitted from the implant to the external device.

According to some embodiments of the first part of the aspect 244SE said reset function is configured to be operated by said magnetic force being applied at least two times.

According to some embodiments of the first part of the aspect 244SE the first energy receiver is configured to receive energy conductively or inductively.

According to some embodiments of the first part of the aspect 244SE the reset function is configured to be triggered if the first energy receiver is receiving energy.

According to some embodiments of the first part of the aspect 244SE the first control program is configured to be running, powered by conductively or inductively received energy.

According to some embodiments of the first part of the aspect 244SE said amount of energy received via the RFID pulse is encoded in a variable pulse feedback signal provided by the feedback unit.

According to some embodiments of the first part of the aspect 244SE the implant comprises at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA, an implant replacing an organ of the patient or part of an organ of the patient or the function thereof,
a vascular treatment device, and
an implant adapted to move fluid inside the body of the patient.

According to some embodiments of the first part of the aspect 244SE the implant comprises an internal control unit adapted to be involved in at least a part of the actions performed by the implant in at least a part of any one of the other embodiments of the aspect 244SE.

### Aspect 245SE 2-Part Key—Encrypted Communication Between Implant and External Device—Embodiments of the Second Aspect of the Disclosure

In a first part of aspect 245SE, a method of communication between an external device and an implant is provided. The method is performed when the implant is implanted in a patient and the external device positioned external to the body of the patient. The external device is adapted to be in electrical connection with the implant, using the body as a conductor. The implant and the external device each comprise a wireless transceiver. The method comprising, confirming the electrical connection between the implant and the external device, transmitting data from the external device to the implant wirelessly or through the electrical connection, and, as a result of the confirmation, using the received data for instructing the implant.

According to some embodiments of the first part of aspect 245SE, the step of transmitting data from the external device to the implant wirelessly comprises transmitting encrypted data wirelessly.

According to some embodiments of the first part of aspect 245SE, the method further comprising: transmitting a key from the external device to the implant using the electrical connection, receiving the key at the implant, and using the key for decrypting the encrypted data.

According to some embodiments of the first part of aspect 245SE, the method further comprising: transmitting a second key from the external device to the implant wirelessly, receiving the second key at the implant, deriving a combined key from the key and second key, and decrypting the encrypted data using the combined key.

According to some embodiments of the first part of aspect 245SE, the method further comprising: transmitting a third key from a second external device, separate from the external device, to the implant wirelessly, receiving the third key at the implant, deriving a combined key from the key and the third key, and decrypting the encrypted data using the combined key.

According to some embodiments of the first part of aspect 245SE, the method further comprising: transmitting a third key from a second external device, separate from the external device, to the implant wirelessly, receiving the third key at the implant, deriving a combined key from the key, the second key and the third key, and decrypting the encrypted data using the combined key.

According to some embodiments of the first part of aspect 245SE, the external device is a wearable external device.

According to some embodiments of the first part of aspect 245SE, the external device is a handset.

According to some embodiments of the first part of aspect 245SE, the second external device is a handset.

According to some embodiments of the first part of aspect 245SE, the second external device is a server.

According to some embodiments of the first part of aspect 245SE, the second external device is cloud based.

According to some embodiments of the first part of aspect 245SE, the step of transmitting a third key from a second external device, separate from the external device, to the implant wirelessly, comprises routing the third key through the external device.

According to some embodiments of the first part of aspect 245SE, the step of transmitting data comprises transmitting data comprising operation instructions to the implant.

According to some embodiments of the first part of aspect 245SE, the method further comprises using the received data to perform at least one of the steps of: updating a control program running in the implant, and operating the implant using the operation instructions.

According to some embodiments of the first part of aspect 245SE, the method further comprises placing a conductive member, configured to be in connection with the external device, in electrical connection with a skin of the patient for conductive communication with the implant.

According to some embodiments of the first part of aspect 245SE the step of transmitting data from the external device to the implant comprises: performing data transmission through the electrical connection involving at least one of transmitting: encrypted data, pulses, positive or negative transients, different frequencies, and using a capacitive coupling.

In a second part of aspect 245SE, an implant adapted for communication with an external device when implanted in a patient is provided. The implant comprises: a first internal transceiver configured to be in electrical connection with the external device, using the body as a conductor, a wireless receiver configured to receive wireless communication from the external device to receive data from the external device. The implant further comprises a computing unit configured to: confirm the electrical connection between the external device and the first internal transceiver and accept wireless communication from the external device on the basis of the confirmation.

According to some embodiments of the second part of aspect 245SE, the wireless receiver is configured to receive wireless communication comprising encrypted data, and wherein the computing unit is further configured to decrypt the encrypted data received wirelessly from the external device.

According to some embodiments of the second part of aspect 245SE, the first internal transceiver is further configured to receive a key from the external device, and wherein the computing unit is further configured to use the key for decrypting the encrypted data.

According to some embodiments of the second part of aspect 245SE, the wireless transceiver is further configured to receive a second key from the external device, and wherein the computing unit is further configured to derive a combined key from the key and the second key and use the derived combined key for decrypting the encrypted data.

According to some embodiments of the second part of aspect 245SE, the wireless transceiver is further configured to receive a third key from a second external device, and wherein the computing unit is further configured to derive a combined key from the key and the third key and use the derived combined key for decrypting the encrypted data.

According to some embodiments of the second part of aspect 245SE, the implant comprises a second wireless receiver for receiving wireless communication from a second external device.

According to some embodiments of the second part of aspect 245SE, the computing unit is further configured to use the received data to perform at least one of: update a control program running in the implant, and operate the implant using the operation instructions.

According to some embodiments of the second part of aspect 245SE the implant comprises at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA,
an implant replacing an organ of the patient or part of an organ of the patient or the function thereof,
a vascular treatment device, and
an implant adapted to move fluid inside the body of the patient.

According to some embodiments of the second part of aspect 245SE the implant comprises an internal control unit adapted to be involved in at least a part of the actions performed by the implant in at least a part of any one of the above embodiments of aspect 245SE.

In a third part of aspect 245SE, an external device adapted for communication with an implant when implanted in a patient is provided. The external device comprises: a first external transmitter configured to be in electrical connection with the implant, using the body as a conductor, and a wireless transmitter configured to transmit wireless communication to the implant.

According to some embodiments of the third part of aspect 245SE, the wireless transmitter is configured to transmit wireless communication comprising encrypted data.

According to some embodiments of the third part of aspect 245SE, the first external transmitter is further configured to transmit a key to the implant, the key being a key for decrypting the encrypted data.

According to some embodiments of the third part of aspect 245SE, the wireless transmitter is further configured to transmit a second key to the implant, the second key being configured to be used in combination with the key for decrypting the encrypted data.

According to some embodiments of the third part of aspect 245SE, the external device is further configured to receive secondary wireless communication from a second external device and transmit the secondary wireless communication to the implant.

According to some embodiments of the third part of aspect 245SE, the external device is a wearable external device.

According to some embodiments of the third part of aspect 245SE, the external device is a handset.

In a fourth part of aspect 245SE, there is provided a system comprising the implant and the external device of aspect 245SE.

According to some embodiments of the fourth part of aspect 245SE, wherein the implant is implanted in a patient, the system further comprising a conductive member configured to be in electrical connection with the external device, wherein the conductive member is configured to be placed in electrical connection with a skin of the patient for conductive communication with the implant.

In a fifth part of aspect 245SE, there is provided computer program product of, or adapted to be run on, an external device adapted for communication with an implant when implanted in a patient, the external device comprising:
a first external transmitter configured to be in electrical connection with the implant, using the body as a conductor,
wherein the computer program product is configured to cause a wireless transmitter to transmit wireless communication comprising encrypted data to the implant.

According to some embodiments of the fifth part of aspect 245SE the computer program product is configured to cause the wireless transmitter is to transmit wireless communication comprising encrypted data.

According to some embodiments of the fifth part of aspect 245SE the computer program product is configured to cause the first external transmitter to transmit a key to the implant, the key being a key for decrypting the encrypted data.

According to some embodiments of the fifth part of aspect 245SE the computer program product is configured to cause the wireless transmitter to transmit a second key to the implant, the second key being configured to be used in combination with the key for decrypting the encrypted data.

According to some embodiments of the fifth part of aspect 245SE the computer program product is configured to cause the external to receive secondary wireless communication from a second external device, and transmit data received in the secondary wireless communication to the implant.

In a fifth part of aspect 245SE, there is provided computer program product of, or adapted to be run on an implant, when implanted in a patient, adapted for communication with an external device, the implant comprising:
a first external transmitter configured to be in electrical connection with the implant, using the body as a conductor,
wherein the computer program product is configured to cause a wireless transmitter to transmit wireless communication to the external device.

### Aspect 246SE 3-Part Key—Multi-Party Encrypted Communication Between Implant and External Device—Embodiments of Aspect 246SE of the Disclosure

In first part of aspect 246SE, a method of communication between an external device and an implant is provided. In this method, the implant is implanted in a patient and the external device positioned external to the body of the patient, wherein the implant and the external device each comprise a wireless transceiver. The method comprising:
receiving, at the implant, a first key from an external device,
receiving, at the implant, by a wireless transmission, a second key, the second key being generated by a second external device, separate from the external device or by a another external device being a generator of the second key on behalf of the second external device, the second key being received at the implant from anyone of, the external device, the second external device, and the generator of the second key,
deriving a combined key by combining the first key and the second key with a third key held by the implant,
transmitting, by a wireless transmission, encrypted data from the external device to the implant, and
decrypting the encrypted data, in the implant, using the combined key.

According to some embodiments of the first part of aspect 246SE, the external device is adapted to be in electrical connection with the implant, using the body as a conductor, wherein the implant is receiving the first key using the electrical connection.
receiving, at the implant, a fourth key from a third external device, the third external device being separate from the external device,
deriving a combined key by combining the first, second and fourth key with the third key held by the implant, and
decrypting the encrypted data, in the implant, using the combined key.

According to some embodiments of the first part of aspect 246SE, the encrypted data originates from the second or third external device.

According to some embodiments of aspect 246SE, the method further comprises altering an operation of the implant comprises controlling or switching an active unit of the implant.

According to some embodiments of the first part of aspect 246SE, the method further comprises confirming the electrical connection between the implant and the external device, and as a result of the confirmation, altering an operation of the implant based on the decrypted data.

According to some embodiments of the first part of aspect 246SE, the confirmation of the electrical connection comprises:
measuring a parameter of the patient, by the implant,
measuring the parameter of the patient, by the external device,
comparing the parameter measured by the implant to the parameter measured by the external device, and
authenticating the connection based on the comparison.

According to some embodiments of the first part of aspect 246SE, the method further comprises the steps of:
measuring a parameter of the patient, by the implant,
measuring the parameter of the patient, by the external device,
comparing the parameter measured by the implant to the parameter measured by the external device,
authenticating the connection between the implant and the external device based on the comparison,
as a result of the confirmation, altering an operation of the implant based on the decrypted data.

According to some embodiments of the first part of aspect 246SE, the external device is a wearable external device.

According to some embodiments of the first part of aspect 246SE, the external device is a handset.

According to some embodiments of the first part of aspect 246SE, the second and/or third external device is a handset.

According to some embodiments of the first part of aspect 246SE, the second and/or third external device is a server.

According to some embodiments of the first part of aspect 246SE, the second and/or third external device is cloud based.

According to some embodiments of the first part of aspect 246SE, the first key is routed through the external device from the second external device.

According to some embodiments of the first part of aspect 246SE, the fourth key is routed through the external device from the third external device.

According to some embodiments of the first part of aspect 246SE, the method further comprises at least one of the steps of:
based on the decrypted data, updating a control program running in the implant, and
operating the implant using operation instructions in the decrypted data.

According to some embodiments of aspect 246SE, one or more of the first, second and third key comprises a biometric key.

In a second part of aspect 246SE, there is provided a method for encrypted communication between an external device and an implant, the method comprising:
receiving, at the external device, a first key, the first key being generated by a second external device, separate from the external device or by another external device being a generator of the second key on behalf of the second external device, the first key being received from anyone of the second external device and the generator of the second key,
receiving, at the external device, a second key from the implant,
deriving a combined key by combining the first key and the second key with a third key held by the external device,
transmitting encrypted data from the implant to the external device, and
decrypting the encrypted data, in the external device, using the combined key.

According to some embodiments of the second part of aspect 246SE, the method further comprises:
receiving, at the external device, a fourth key from a third external device, the third external device being separate from the external device,
deriving a combined key by combining the first, second and fourth key with the third key held by the external device, and
decrypting the encrypted data, in the external device, using the combined key.

According to some embodiments of the second part of aspect 246SE, the external device is a wearable external device.

According to some embodiments of the second part of aspect 246SE, the external device is a handset.

According to some embodiments of the second part of aspect 246SE, the second and/or third external device is a handset.

According to some embodiments of the second part of aspect 246SE, the second and/or third external device is a server.

According to some embodiments of the second part of aspect 246SE, the second and/or third external device is cloud based.

According to some embodiments of the second part of aspect 246SE, one or more of the first, second and third key comprises a biometric key.

According to some embodiments of the second part of aspect 246SE, the method further comprising authentication of the communication between the implant and the external device comprising the steps of:
measuring a parameter of the patient, by the implant,
measuring the parameter of the patient, by the external device,
comparing the parameter measured by the implant to the parameter measured by the external device, and
authenticating the connection between the implant and the external device based on the comparison,
as a result of authentication, decrypting the encrypted data, in the external device, using the combined key.

According to some embodiments of the first or second part of aspect 246SE, the method further comprises placing a conductive member, configured to be in connection with the external device, in electrical connection with a skin of the patient for conductive communication with the implant.

In a third part of aspect 246SE, there is provided an implant adapted for communication with an external device, when the implant is implanted in a patient, the implant comprising: a wireless transceiver configured to receive wireless communication, and configured for:
receiving a first key from the external device,
receiving a second key, the second key being generated by a second external device, separate from the external device or by a another external device being a generator of the second key on behalf of the second external device, the second key being received at the implant from anyone of, the external device, the second external device, and the generator of the second key, from the external device,
receiving encrypted data,

The implant further comprising a computing unit configured for:
deriving a combined key by combining the first and second keys with a third key held by the implant,
decrypting the encrypted data using the combined key.

According to some embodiments of the third part of aspect 246SE, the wireless transceiver is configured for:
receiving a fourth key from a third external device,
wherein the computing unit is configured for:
deriving a combined key by combining the first, second and fourth key with the third key held by the implant, and
decrypting the encrypted data using the combined key.

According to some embodiments of the third part of aspect 246SE, the computing unit is configured for altering an operation of the implant based on the decrypted data.

According to some embodiments of the third part of aspect 246SE, the computing unit is configured for controlling or switching an active unit of the implant.

According to some embodiments of the third part of aspect 246SE, the computing unit is configured for: confirming a connection between the implant and the external device, and as a result of the confirmation, altering an operation of the implant based on the decrypted data.

According to some embodiments of the third part of aspect 246SE, the confirmation of the electrical connection comprises:
measuring a parameter of the patient, by the implant,
receiving a measured parameter of the patient, from the external device,
comparing the parameter measured by the implant to the parameter measured by the external device, and
performing confirmation of the connection based on the comparison.

According to some embodiments of the third part of aspect 246SE, the computing unit is configured for at least one of: based on the decrypted data, updating a control program running in the implant, and operating the implant using operation instructions in the decrypted data.

According to some embodiments of the third part of aspect 246SE, the third key comprises a biometric key.

In a fourth part of aspect 246SE, there is provided an external device adapted for communication with an implant, when the implant is implanted in a patient, the external device comprising a wireless transceiver configured to receive wireless communication, and configured for:
receiving a first key, the first key being generated by a second external device, separate from the external device or by another external device being a generator of the second key on behalf of the second external device, the first key being received from anyone of the second external device and the generator of the second key,
receiving a second key from the implant,
receiving encrypted data from the implant,

The external device further comprises a computing unit configured for:
deriving a combined key by combining the first and second keys with a third key held by the external device,
decrypting the encrypted data using the combined key.

According to some embodiments of the fourth part of aspect 246SE, the wireless transceiver is configured for:
receiving a fourth key from a third external device,
wherein the computing unit is configured for:
deriving a combined key by combining the first, second and fourth key with the third key held by the external device, and
decrypting the encrypted data using the combined key.

According to some embodiments of the fourth part of aspect 246SE, the external device is a wearable external device.

According to some embodiments of the fourth part of aspect 246SE, the external device is a handset.

According to some embodiments of the fourth part of aspect 246SE, the computing unit is configured to confirm the communication between the implant and the external device, wherein the confirmation comprises:
measuring a parameter of the patient, by the external device,
receiving a measured parameter of the patient, from the implant,
comparing the parameter measured by the implant to the parameter measured by the external device,
performing confirmation of the connection based on the comparison, and
as a result of the confirmation, decrypting the encrypted data, in the external device, using the combined key.

According to some embodiments of the fourth part of aspect 246SE, the third key comprises a biometric key.

In a fifth part of aspect 246SE, there is provided a system comprising an implant according to the third part of aspect 246SE and an external device according to the fourth part of aspect 246SE, wherein the implant is implanted in a patient, the system further comprising a conductive member configured to be in electrical connection with the external device, wherein the conductive member is configured to be placed in electrical connection with a skin of the patient for conductive communication with the implant.

In a sixth part of aspect 246SE, there is provided a computer program product comprising a computer-readable storage medium with instructions adapted to carry out the method the first or second part of aspect 246SE, and/or with instructions adapted to carry out anyone of the implant actions from of aspect 246SE, when executed by an external device having processing capability.

According to some embodiments of the first part of aspect 246SE the first key is received at the implant from the external device, by a wireless transmission.

According to some embodiments of the first part of aspect 246SE the first key is transmitted by the external device.

According to some embodiments of the third part of aspect 246SE the encrypted data is received from the external device or the second external device or another external device via the internet.

According to some embodiments of the third part of aspect 246SE the third external device is a server comprising a database, the database comprising data pertaining to control program updates and/or instructions.

According to some embodiments of the third part of aspect 246SE the database may communicate with a caregiver and/or the implant According to some embodiments of the third part of aspect 246SE the database may communicate with a caregiver and/or the implant via the external device.

According to some embodiments of the third part of aspect 246SE the implant comprises at least one of
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA,
an implant replacing an organ of the patient or part of an organ of the patient or the function thereof,
a vascular treatment device, and
an implant adapted to move fluid inside the body of the patient.

In a seventh part of aspect 246SE, there is provided a computer program product of, or adapted to be run on, an external device adapted for communication with an implant, when the implant is implanted in a patient, the external device comprising:
a. a wireless transceiver configured to receive wireless communication, wherein the computer program product is configured to cause the wireless transceiver to:
   i. receive a first key, the first key being generated by a second external device, separate from the external device or by another external device being a generator of the second key on behalf of the second external device, the first key being received from anyone of the second external device and the generator of the second key,
   ii. receive a second key from the implant,
   iii. receive encrypted data from the implant,
b. a computing unit, wherein the computer program product is configured cause the computing unit to:
   i. derive a combined key by combining the first and second keys with a third key held by the external device,
   ii. decrypt the encrypted data using the combined key.

According to some embodiments of the seventh part of aspect 246SE the computer program product is configured to cause the wireless transceiver to:
a. receive a fourth key from a third external device,
wherein the computing unit is configured to:
b. derive a combined key by combining the first, second and fourth key with the third key held by the external device, and
c. decrypt the encrypted data using the combined key.

According to some embodiments of the seventh part of aspect 246SE the computer program product is configured to cause the computing unit to confirm the communication between the implant and the external device, wherein the confirmation comprises:
a. measuring a parameter of the patient, by the external device,
b. receiving a measured parameter of the patient, from the implant,
c. comparing the parameter measured by the implant to the parameter measured by the external device,
d. performing confirmation of the connection based on the comparison, and
e. as a result of the confirmation, decrypting the encrypted data, in the external device, using the combined key.

According to some embodiments of the seventh part of aspect 246SE the third key comprises a biometric key.

In an eighth part of aspect 246SE, there is provided a computer program product adapted to be run on, an implant adapted for communication with an external device, when the implant is implanted in a patient, the implant comprising:
a. a wireless transceiver configured to receive wireless communication, wherein the computer program product is configured to cause the wireless transceiver to:
   i. receive a first key, the first key being generated by a second external device, separate from the external device or by another external device being a generator of the second key on behalf of the second external device, the first key being received from anyone of the second external device and the generator of the second key,
   ii. receive a second key from the external device,
   iii. receive encrypted data from the external device,
b. a computing unit, wherein the software is configured to cause the computing unit to:
   i. derive a combined key by combining the first and second keys with a third key held by the implant,
   ii. decrypt the encrypted data using the combined key.

According to some embodiments of the eighth part of aspect 246SE the computer program product is configured to cause the wireless transceiver to:
a. receive a fourth key from a third external device,
wherein the computing unit is configured to:
b. derive a combined key by combining the first, second and fourth key with the third key held by the external device, and
c. decrypt the encrypted data using the combined key.

According to some embodiments of the eighth part of aspect 246SE the computer program product is configured to cause the computing unit to confirm the communication between the implant and the external device, wherein the confirmation comprises:
a. measuring a parameter of the patient, by the external device,
b. receiving a measured parameter of the patient, from the implant,
c. comparing the parameter measured by the implant to the parameter measured by the external device,
d. performing confirmation of the connection based on the comparison, and
e. as a result of the confirmation, decrypting the encrypted data, in the external device, using the combined key.

According to some embodiments of the eighth part of aspect 246SE the third key comprises a biometric key.

In a ninth part of aspect 246SE, there is provided a computer program product comprising a computer-readable storage medium with instructions adapted to carry out the method of the aspect 244SE and/or with instructions adapted to carry out anyone of the implant actions of aspect 245SE, when executed by an implant having processing capability.

According to some embodiments of the second part of aspect 246SE, and/or with ability to use any of the methods of the first part of aspect 246SE, and/or with ability to communicate with an external device of the third part of aspect 246SE, and or with ability to use anyone of the computer program product of the sixth, eighth, or ninth parts of aspect 246SE.

In a tenth part of aspect 246SE, there is provided a method for communication between an external device and an implant, when the implant is implanted in a patient and the external device positioned external to the body of the patient, wherein the implant and the external device each comprise a wireless transceiver, the method comprising:
receiving, at the implant, a first key from an external device,
deriving a combined key by combining the first key and a key held by the implant,
transmitting, by a wireless or electrical transmission, encrypted data from the external device to the implant, and
decrypting the encrypted data, in the implant, using the combined key.

### Aspect 247SE Electrical Connection—Conductive Member in Electrical Connection with the External Device—Embodiments of Aspect 247SE of the Disclosure

In a first part of aspect 247SE, a system for communication between an external device and an implant implanted in a patient is provided. The system comprises a conductive member configured to be in connection with the external device, the conductive member being configured to be placed in electrical connection with a skin of the patient for electrical or conductive communication with the implant.

According to some embodiments of the first part of aspect 247SE, the conductive member comprises a conductive interface for connecting the conductive member to the external device.

According to some embodiments of the first part of aspect 247SE, the external device is configured to transmit a conductive communication to the implant.

According to some embodiments of the first part of aspect 247SE, the implant is configured to transmit a conductive communication to the external device.

According to some embodiments of the first part of aspect 247SE, the external device and/or the conductive member comprises a verification unit configured to receive authentication input from a user, for authenticating the conductive communication between the implant and the external device.

According to some embodiments of the first part of aspect 247SE, the authentication input is a code.

According to some embodiments of the first part of aspect 247SE, the authentication input is based on a biometric technique selected from the list of: a fingerprint, a palm vein structure, image recognition, face recognition, iris recognition, a retinal scan, a hand geometry, and genome comparison.

According to some embodiments of the first part of aspect 247SE, the conductive member comprises a fingerprint reader, wherein the verification unit is configured to receive a fingerprint from the conductive member. Any other means for collecting biometric data is equally possible.

According to some embodiments of the first part of aspect 247SE, the implant comprises: a first sensor for measuring a parameter of the patient, by the implant, and an internal computing unit configured for:
receiving a parameter of the patient, from the external device,
comparing the parameter measured by the implant to a parameter measured by the external device, and
performing authentication of the conductive communication based on the comparison.

According to some embodiments of the first part of aspect 247SE, the implant being connected to a sensation generator, the implant being configured for:
storing authentication data, related to a sensation generated by the sensation generator,
receiving input authentication data from the external device, and wherein the implant comprises an internal computing unit configured for:
comparing the authentication data to the input authentication data, and
performing authentication of the conductive communication based on the comparison.

According to some embodiments of the first part of aspect 247SE, the external device is a handset or a wearable device.

According to some embodiments of the first part of aspect 247SE, the conductive communication comprises a key or a part of the key to be used for decrypting encrypted data received by the external device or the implant.

According to some embodiments of the first part of aspect 247SE, the external device is configured to transmit a first part of the key to the implant using the conductive communication, and to wirelessly transmit a second part of the key to the implant, wherein the implant is adapted to decrypt the encrypted data, using a combined key derived from the received first and second parts of the key.

According to some embodiments of the first part of aspect 247SE, the implant comprises an internal computing unit configured to operate the implant using operation instructions, wherein the conductive communication comprises instructions for operating the implant. In some embodiments, the operation of the implant is only conducted upon positive authentication of the conductive communication as described above.

According to some embodiments of the first part of aspect 247SE, the implant comprises an internal computing unit configured to update a control program running in the implant, wherein the conductive communication comprises instructions for updating the control program. In some embodiments, the updating of the control program of the implant is only conducted upon positive authentication of the conductive communication as described above.

According to some embodiments of the first part of aspect 247SE, the conductive communication comprises feedback parameters relating to functionality of the implant.

According to some embodiments of the first part of aspect 247SE, the implant comprises a sensor for sensing at least one physiological parameter of the patient, wherein the conductive communication comprises said at least one physiological parameter of the patient.

In a second part of aspect 247SE, a method for communication between an external device and an implant implanted in a patient is provided. The method comprises placing a conductive member, configured to be in connection with the external device, in electrical connection with a skin of the patient for conductive communication with the implant.

According to some embodiments of the second part of aspect 247SE, the conductive member comprises a conductive interface for connecting the conductive member to the external device.

According to some embodiments of the second part of aspect 247SE, the method comprises transmitting a conductive communication to the implant by the external device.

According to some embodiments of the second part of aspect 247SE, the method comprises transmitting a conductive communication to the external device by the implant.

According to some embodiments of the second part of aspect 247SE, the method comprises receiving of an authentication input from a user by a verification unit of the external device and authenticating the conductive communication between the implant and the external device using the authentication input.

According to some embodiments of the second part of aspect 247SE, the authentication input is a code.

According to some embodiments of the second part of aspect 247SE, the authentication input is based on a biometric technique selected from the list of: a fingerprint, a palm vein structure, image recognition, face recognition, iris recognition, a retinal scan, a hand geometry, and genome comparison.

According to some embodiments of the second part of aspect 247SE, the conductive member comprises a fingerprint reader, wherein the method comprises receiving a fingerprint from the conductive member by the verification unit.

According to some embodiments of the second part of aspect 247SE, the method comprises measuring a parameter of the patient by a sensor of the implant, receiving, by an internal computing unit of the implant, a parameter of the patient from the external device, comparing, by the internal computing unit of the implant, the parameter measured by the implant to the parameter measured by the external device, and performing, by the internal computing unit of the implant, authentication of the conductive communication based on the comparison.

According to some embodiments of the second part of aspect 247SE, the method comprises: generating, by a sensation generator, a sensation detectable by a sense of the patient, storing, by the implant, authentication data, related to the generated sensation, providing, by the patient, input to the external device, resulting in input authentication data, and authenticating the conductive communication based on a comparison of the input authentication data and the authentication data.

According to some embodiments of the second part of aspect 247SE, the external device is a handset or a wearable device.

According to some embodiments of the second part of aspect 247SE, the conductive communication comprises a key or a part of the key to be used for decrypting encrypted data received by the external device or the implant.

According to some embodiments of the second part of aspect 247SE, the method comprises: transmitting, by the external device, a first part of the key to the implant using the conductive communication, transmitting, by the external device, a second part of the key to the implant using a wireless connection, deriving a combined key from the received first and second parts of the key, and decrypting, by the implant, the encrypted data, using the combined key.

According to some embodiments of the second part of aspect 247SE, the method comprises operating the implant using operation instructions, by an internal computing unit of the implant, wherein the conductive communication comprises instructions for operating the implant.

According to some embodiments of the second part of aspect 247SE, the method comprises updating a control program running in the implant, by an internal computing unit of the implant, wherein the conductive communication comprises instructions for updating the control program.

According to some embodiments of the second part of aspect 247SE, the conductive communication comprises feedback parameters relating to functionality of the implant.

According to some embodiments of the second part of aspect 247SE, the method comprises sensing of at least one physiological parameter of the patient, by a sensor of the implant, wherein the conductive communication comprises said at least one physiological parameter of the patient.

In a third part of aspect 247SE, an implant implanted in a patient is provided. The implant comprises an internal computing unit configured to operate the implant based on an authentication input and/or using operating instructions, wherein the authentication input and/or the operating instructions are received by conductive communication with an external device.

According to some embodiments of the third part of aspect 247SE, the internal computing unit is further configured to update a control program running in the implant, wherein the conductive communication comprises instructions for updating the control program.

According to some embodiments of the third part of aspect 247SE, the implant further comprising a sensor for measuring a parameter of the patient and wherein the internal computing unit is further configured for:
receiving a parameter of the patient, from the external device,
comparing the parameter measured by the implant to a parameter measured by the external device,
performing authentication of the conductive communication based on the comparison; and upon an authenticated conductive communication, operating the implant using the operating instructions.

According to some embodiments of the third part of aspect 247SE, the implant being connected to a sensation generator, the implant being configured for:
storing authentication data, related to a sensation generated by the sensation generator,
receiving input authentication data from the external device, and
wherein the implant comprises an internal computing unit configured for:
comparing the authentication data to the input authentication data, and
performing authentication of the conductive communication based on the comparison,
upon an authenticated conductive communication, operating the implant using the operating instructions.

According to some embodiments of the third part of aspect 247SE, the implant further comprising a sensor for sensing at least one physiological parameter of the patient, wherein the conductive communication comprises said at least one physiological parameter of the patient.

In a fourth part of aspect 247SE, an external device adapted for communication with an implant, when the implant is implanted in a patient, is provided. The external device is configured to be placed in electrical connection with a conductive member, for conductive communication with the implant.

According to some embodiments of the fourth part of aspect 247SE, the external device comprises a conductive interface for connecting with the conductive member.

According to some embodiments of the fourth part of aspect 247SE, the external device being configured to transmit a conductive communication to the implant when in electrical connection with the conductive member.

According to some embodiments of the fourth part of aspect 247SE, the external device being configured to receive conductive communication from the implant when in electrical connection with the conductive member.

According to some embodiments of the fourth part of aspect 247SE, the external device comprising a verification unit configured to receive authentication input from a user, for authenticating the conductive communication between the implant and the external device.

According to some embodiments of the fourth part of aspect 247SE, the external device being a handset or a wearable device.

According to some embodiments of the fourth part of aspect 247SE, the conductive communication comprises a key or a part of the key to be used for decrypting encrypted data received by the external device or the implant.

According to some embodiments of the fourth part of aspect 247SE, the external device is configured to transmit a first part of the key to the implant using the conductive communication, and to wirelessly transmit a second part of the key to the implant, and to encrypt data to be sent to the implant such that it can be decrypted using a combined key derived from the first and second parts of the key.

According to some embodiments of the fourth part of aspect 247SE, the external device being configured to transmit instructions for updating a control program of the implant using the conductive communication.

According to some embodiments of the fourth part of aspect 247SE, the external device being configured to transmit operation instructions of the implant using the conductive communication.

In a fifth part of aspect 247SE, a conductive member configured to be in connection with an external device for communication between the external device and an implant implanted in a patient is provided. The conductive member being configured to be in electrical connection with a skin of the patient or any other part of a body of the patient.

According to some embodiments of the fifth part of aspect 247SE, the conductive member comprising a conductive interface for connecting the conductive member to the external device.

According to some embodiments of the fifth part of aspect 247SE, the conductive member comprising a fingerprint reader, wherein the conductive member is configured to transmit a fingerprint read by the fingerprint reader to the external device.

According to some embodiments of the fifth part of aspect 247SE, the conductive member being in the form of a case of the external device, the case comprising a capacitive area configured to be in electrical connection with a skin of the patient.

According to some embodiments of the fifth part of aspect 247SE, the external device is a mobile phone, wherein the conductive member is in the form of a mobile phone case.

According to some embodiments of the fifth part of aspect 247SE the conductive member is arranged as an arm or wrist band being integrally formed with, or connected to, the external device.

According to some embodiments of the first part of aspect 247SE the conductive member is configured to be in conductive or electrical connection with the external device.

According to some embodiments of the first part of aspect 247SE the conductive member is configured to be in wireless connection with the external device.

According to some embodiments of the first part of aspect 247SE the conductive member is configured to be a screen of the external device, the screen being configured to receive data using electric charge.

According to some embodiments of the first part of aspect 247SE wherein the conductive member comprises the verification unit.

According to some embodiments of the first part of aspect 247SE the external device comprises the verification unit.

According to some embodiments of the first part of aspect 247SE wherein the establishment of conductive communication is configured to authenticate or partially authenticate the conductive communication between the implant and the external device.

According to some embodiments the implant of the third part of aspect 247SE, and/or with ability to use any of the methods of the second part of aspect 247SE, and/or with ability to be part of any of the systems of the first part of aspect 247SE, and/or with ability to communicate via the conductive member according to any of the fifth part of aspect 247SE, and/or with ability to communicate with the external device of the fourth part of aspect 247SE, and/or with ability to use the computer program product of the sixth part of aspect 247SE, and/or with ability to use an internal control unit, wherein the implant comprises at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA,
an implant replacing an organ of the patient or part of an organ of the patient or the function thereof,
a vascular treatment device, and
an implant adapted to move fluid inside the body of the patient.

According to some embodiments of the fourth part of aspect 247SE the external device is a smartwatch.

In a sixth part of aspect 247SE, there is provided computer program product of, or adapted to be run on, an external device adapted for communication with an implant, when the implant is implanted in a patient, wherein the external device is configured to be placed in electrical connection with a conductive member, wherein the computer program product is configured to cause the conductive member to have conductive communication with the implant.

According to some embodiments of the sixth part of aspect 247SE the computer program product is configured to cause the external device to transmit a conductive communication to the implant when in electrical connection with the conductive member.

According to some embodiments of the sixth part of aspect 247SE the computer program product is configured to cause the external device to receive conductive communication from the implant when in electrical connection with the conductive member.

According to some embodiments of the sixth part of aspect 247SE the computer program product is configured to cause a verification unit of the external device to receive authentication input from a user, for authenticating the conductive communication between the implant and the external device.

According to some embodiments of the sixth part of aspect 247SE the conductive communication comprises a key or a part of the key to be used for decrypting encrypted data received by the external device or the implant.

According to some embodiments of the sixth part of aspect 247SE the computer program product is configured to cause the external device to transmit a first part of the key to the implant using the conductive communication, and to wirelessly transmit a second part of the key to the implant, and to encrypt data to be sent to the implant such that it can be decrypted using a combined key derived from the first and second parts of the key.

According to some embodiments of the sixth part of aspect 247SE the computer program product is configured to cause the external device to transmit instructions for updating a control program of the implant using the conductive communication.

According to some embodiments of the sixth part of aspect 247SE the computer program product is configured to cause the external device to transmit operation instructions of the implant using the conductive communication.

In a seventh part of aspect 247SE, there is provided computer program product of, or adapted to be run on, an implant adapted for communication with an external device adapted to be placed in electrical connection with a conductive member, when the implant is implanted in a patient, wherein the computer program product used by a computing unit on the implant is configured to cause the implant to have communication with the conductive member using the body as a signal transmitter.

According to some embodiments of the seventh part of aspect 247SE the computer program product is configured to cause the implant to transmit a conductive communication to the external device when in electrical connection with the conductive member.

According to some embodiments of the seventh part of aspect 247SE the computer program product is configured to cause the implant to receive conductive communication from the external device when in electrical connection with the conductive member.

According to some embodiments of the seventh part of aspect 247SE the computer program product is configured to cause a verification unit of the implant to receive authentication input from a user, for authenticating the conductive communication between the implant and the external device.

According to some embodiments of the seventh part of aspect 247SE the conductive communication comprises a key or a part of the key to be used for decrypting encrypted data received by the implant.

According to some embodiments of the seventh part of aspect 247SE the computer program product is configured to receive from the external device a first part of the key to the implant using the conductive communication, and to receive wirelessly a second part of the key to the implant, and to encrypt data sent to the implant such that it can be decrypted using a combined key derived from the first and second parts of the key.

According to some embodiments of the seventh part of aspect 247SE the computer program product is configured to receive instructions for updating a control program at the implant from the external device using the conductive communication.

According to some embodiments of the seventh part of aspect 247SE the computer program product is configured to receive operation instructions at the implant from the external device using the conductive communication.

According to some embodiments of the first part of aspect 247SE the external device is configured to transmit a conductive communication to the implant.

According to some embodiments of the first part of aspect 247SE the implant is configured to transmit a conductive communication to the external device.

According to some embodiments of the first part of aspect 247SE the external device and/or the conductive member comprises a verification unit configured to receive authentication input from a user, for authenticating the conductive communication between the implant and the external device.

According to some embodiments of the first part of aspect 247SE the authentication input is a code.

According to some embodiments of the first part of aspect 247SE the authentication input is based on a biometric technique selected from the list of: a fingerprint, a palm vein structure, image recognition, face recognition, iris recognition, a retinal scan, a hand geometry, and genome comparison.

According to some embodiments of the first part of aspect 247SE the implant comprises:
a. a sensor for measuring a parameter of the patient, by the implant
b. an internal computing unit configured for:
   i. receiving a parameter of the patient, from the external device,
   ii. comparing the parameter measured by the implant to the parameter measured by the external device, and
   iii. performing authentication of the conductive communication based on the comparison

According to some embodiments of the first part of aspect 247SE the implant being connected to a sensation generator, the implant being configured for:
a. storing authentication data, related to a sensation generated by the sensation generator,
b. receiving input authentication data from the external device, and wherein the implant comprises an internal computing unit configured for:
   i. comparing the authentication data to the input authentication data, and
   ii. performing authentication of the conductive communication based on the comparison.

According to some embodiments of the first part of aspect 247SE the external device is a handset or a wearable device.

According to some embodiments of the first part of aspect 247SE the conductive communication comprises a key or a part of the key to be used for decrypting encrypted data received by the external device or the implant.

According to some embodiments of the first part of aspect 247SE the external device is configured to transmit a first part of the key to the implant using the conductive communication, and to wirelessly transmit a second part of the key to the implant, wherein the implant is adapted to decrypt the encrypted data, using a combined key derived from the received first and second parts of the key.

According to some embodiments of the first part of aspect 247SE the implant comprises an internal computing unit configured to operate the implant using operation instructions, wherein the conductive communication comprises instructions for operating the implant.

According to some embodiments of the first part of aspect 247SE the implant comprises an internal computing unit configured to update a control program running in the implant, wherein the conductive communication comprises instructions for updating the control program.

According to some embodiments of the first part of aspect 247SE the conductive communication comprises feedback parameters relating to functionality of the implant.

According to some embodiments of the first part of aspect 247SE the implant comprises a sensor for sensing at least one physiological parameter of the patient, wherein the conductive communication comprises said at least one physiological parameter of the patient.

According to some embodiments, the implant according to the third part of aspect 247SE, and/or with ability to use any of the methods of the second part of aspect 247SE, and/or with ability to be part of any system of the first part of aspect 247SE, and/or with ability to communicate via the conductive member of the fifth part of aspect 247SE, and/or with ability to communicate with the external device of the fourth part of aspect 247SE, and/or with ability to use the computer program product of the sixth or seventh parts of aspect 247SE, comprising an internal control unit adapted to be involved in at least a part of the actions performed by the implant in at least a part of any one of the other embodiments.

### Aspect 248SE Device Synchronization Sensation—Authenticating a Connection Between an Implant and the External Device by Generating Sensations—Embodiments of Aspect 248SE of the Disclosure

In a first part of aspect 248SE, there is provided a method of authenticating a connection between an implant implanted in a patient, and an external device. The method comprising:
generating, by a sensation generator, a sensation detectable by a sense of the patient,
storing, by the implant, authentication data, related to the generated sensation,
providing, by the patient, input to the external device, resulting in input authentication data, and
authenticating the connection based on an analysis of the input authentication data and the authentication data.

According to some embodiments of the first part of aspect 248SE the method further comprises the step of communicating further data between the implant and the external device following positive authentication.

According to some embodiments of the first part of aspect 248SE authentication data comprises a timestamp of the sensation and wherein the input authentication data comprises a timestamp of the input from the patient.

According to some embodiments of the first part of aspect 248SE authenticating the connection comprises: calculating a time difference between the timestamp of the sensation and the timestamp of the input from the patient, and upon determining that the time difference is less than a threshold, authenticating the connection

According to some embodiments of the first part of aspect 248SE authentication data comprises a number of times that the sensation is generated by the sensation generator, and wherein the input authentication data comprises an input from the patient relating to a number of times the patient detected the sensation, wherein authenticating the connection comprises: upon determining that the number of times of the authentication data and the input authentication data are equal, authenticating the connection.

According to some embodiments of the first part of aspect 248SE the sensation comprises a plurality of sensation components.

According to some embodiments of the first part of aspect 248SE the sensation or sensation components comprise a vibration.

According to some embodiments of the first part of aspect 248SE the sensation or sensation components comprise a sound.

According to some embodiments of the first part of aspect 248SE the sensation or sensation components comprise a photonic signal.

According to some embodiments of the first part of aspect 248SE the sensation or sensation components comprise a light signal.

According to some embodiments of the first part of aspect 248SE the sensation or sensation components comprise an electric signal.

According to some embodiments of the first part of aspect 248SE the sensation or sensation components comprise a heat signal.

According to some embodiments of the first part of aspect 248SE the sensation generator is contained within the implant.

According to some embodiments of the first part of aspect 248SE the communication between the implant and the external device is a wireless communication.

According to some embodiments of the first part of aspect 248SE the communication between the implant and the external device is a conductive communication.

According to some embodiments of the first part of aspect 248SE the method further comprises the step of:
transmitting the input authentication data from the external device to the implant,
wherein the analysis is performed by the implant.

According to some embodiments of the first part of aspect 248SE the method further comprises the step of:
transmitting the authentication data from the implant to the external device,
wherein the analysis is performed by the external device.

According to some embodiments of the first part of aspect 248SE the implant comprises a motor for controlling a physical function in the body of the patient, wherein the motor being the sensation generator.

According to some embodiments of the first part of aspect 248SE the sensation is a vibration created by running the motor.

According to some embodiments of the first part of aspect 248SE the sensation is a sound created by running the motor.

According to some embodiments of the first part of aspect 248SE the analysis is performed by the implant, the method further comprising the step of:
continuously requesting by the external device, or receiving at the external device, information of an authentication status of the connection between the implant and the external device, and upon determining, at the external device, that the connection is authenticated, transmitting further data from the external device to the implant.

According to some embodiments of the first part of aspect 248SE the further data comprises at least one of:
data for updating a control program running in the implant, and
operation instructions for operating the implant.

According to some embodiments of the first part of aspect 248SE the analysis is performed by the external device, the method further comprising the step of:
continuously requesting by the implant, or receiving at the implant, information of an authentication status of the connection between the implant and the external device, and upon determining, at the implant, that the connection is authenticated, transmitting further data from the implant to the external device.

According to some embodiments of the first part of aspect 248SE the further data comprises data sensed by a sensor connected to the implant.

In a second part of aspect 248SE, there is provided an implant, implanted in a patient, adapted for connection with an external device, the implant connected to a sensation generator, the implant being configured for: storing authentication data, related to a sensation generated by the sensation generator,
receiving input authentication data from the external device, and wherein the implant comprises an internal computing unit configured for:
analyzing the authentication data and the input authentication data, and
performing authentication of the connection based on the analysis.

According to some embodiments of the second part of aspect 248SE the implant is further configured for communicating further data to the external device following positive authentication.

According to some embodiments of the second part of aspect 248SE the authentication data comprises a timestamp of the sensation and wherein the input authentication data comprises a timestamp of the input from the patient.

According to some embodiments of the second part of aspect 248SE authenticating the connection comprises: calculating a time difference between the timestamp of the sensation and the timestamp of the input from the patient, and upon determining that the time difference is less than a threshold, authenticating the connection.

According to some embodiments of the second part of aspect 248SE the authentication data comprises a number of times that the sensation is generated by the sensation generator, and wherein the input authentication data comprises an input from the patient relating to a number of times the patient detected the sensation, wherein authenticating the connection comprises: upon determining that the number of times of the authentication data and the input authentication data are equal, authenticating the connection.

According to some embodiments of the second part of aspect 248SE the sensation generator is contained within the implant.

According to some embodiments of the second part of aspect 248SE the sensation generator is configured to create the sensation comprising a plurality of sensation components.

According to some embodiments of the second part of aspect 248SE the sensation generator is configured to create the sensation or sensation components by vibration of the sensation generator.

According to some embodiments of the second part of aspect 248SE the sensation generator is configured to create the sensation or sensation components by playing a sound.

According to some embodiments of the second part of aspect 248SE the sensation generator is configured to create the sensation or sensation components by providing a photonic signal.

According to some embodiments of the second part of aspect 248SE the sensation generator is configured to create the sensation or sensation components by providing a light signal.

According to some embodiments of the second part of aspect 248SE the sensation generator is configured to create the sensation or sensation components by providing an electric signal.

According to some embodiments of the second part of aspect 248SE the sensation generator is configured to create the sensation or sensation components by providing a heat signal.

According to some embodiments of the second part of aspect 248SE the communication between the implant and the external device is a wireless communication.

According to some embodiments of the second part of aspect 248SE the communication between the implant and the external device is a conductive communication.

According to some embodiments of the second part of aspect 248SE the implant comprises a motor for controlling a physical function in the body of the patient, wherein the motor being the sensation generator.

According to some embodiments of the second part of aspect 248SE the sensation is a vibration created by running the motor.

According to some embodiments of the second part of aspect 248SE the sensation is a sound created by running the motor.

In a third part of aspect 248SE, there is provided an external device, adapted for connection with an implant, implanted in a patient, the external device comprising:
an interface for receiving, by the patient, input to the external device, resulting in input authentication data,
a receiver for receiving authentication data from the implant, the authentication data relating to a generated sensation of a sensation generator connected to the implant;
an external computing unit configured for:
i. analyzing the authentication data and the input authentication data, and
ii. performing authentication of the connection based on the analysis.

According to some embodiments of the third part of aspect 248SE the external device is further configured for communicating further data to the implant following positive authentication.

According to some embodiments of the third part of aspect 248SE the authentication data comprises a timestamp and wherein the input authentication data comprises a timestamp of the input from the patient.

According to some embodiments of the third part of aspect 248SE authenticating the connection comprises: calculating a time difference between the timestamp of the authentication data and the timestamp of the input from the patient, and upon determining that the time difference is less than a threshold, authenticating the connection.

According to some embodiments of the third part of aspect 248SE the authentication data comprises a number of times that the sensation is generated by the sensation generator, and wherein the input authentication data comprises an input from the patient relating to a number of times the patient detected the sensation, wherein authenticating the connection comprises: upon determining that the number of times of the authentication data and the input authentication data are equal, authenticating the connection.

According to some embodiments of the third part of aspect 248SE the communication between the implant and the external device is a wireless communication.

According to some embodiments of the third part of aspect 248SE the communication between the implant and the external device is a conductive communication.

According to some embodiments of the third part of aspect 248SE the external device further comprises a conductive member configured to be in electrical connection with the external device, wherein the conductive member is configured to be placed in electrical connection with a skin of the patient for conductive communication with the implant.

According to some embodiments of the first part of aspect 248SE the method further comprises transmitting further data between the implant and the external device, wherein the further data is used or acted upon, only after authentication of the connection is performed.

According to some embodiments of the second part of aspect 248SE the implant comprises at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

In a fourth part of aspect 248SE there is provided a computer program product of, or adapted to be run on, an external device, adapted for connection with an implant, implanted in a patient, the external device comprising:
a. an interface for receiving, by the patient, input to the external device, resulting in input authentication data,
b. a receiver for receiving authentication data from the implant, the authentication data relating to a generated sensation of a sensation generator being part of the implant or external device,
c. an external computing unit, wherein the computer program product is configured to cause the external computing unit to:
   i. analyze the authentication data and the input authentication data, and
   ii. perform authentication of the connection based on the analysis.

According to some embodiments of the fourth part of aspect 248SE the computer program product is configured to cause the external device to communicate further data to the implant following positive authentication.

According to some embodiments of the fourth part of aspect 248SE the authentication data comprises a timestamp and wherein the input authentication data comprises a timestamp of the input from the patient.

According to some embodiments of the fourth part of aspect 248SE authenticating the connection comprises: calculating a time difference between the timestamp of the authentication data and the timestamp of the input from the patient, and upon determining that the time difference is less than a threshold, authenticating the connection.

According to some embodiments of the fourth part of aspect 248SE the authentication data comprises a number of times that the sensation is generated by the sensation generator, and wherein the input authentication data comprises an input from the patient relating to a number of times the patient detected the sensation, wherein authenticating the connection comprises: upon determining that the number of times of the authentication data and the input authentication data are equal, authenticating the connection.

According to some embodiments of the fourth part of aspect 248SE the communication between the implant and the external device is a wireless communication.

According to some embodiments of the fourth part of aspect 248SE the communication between the implant and the external device is a conductive communication.

In a fifth part of aspect 248SE there is provided a computer program product adapted to be run on, an implant, implanted in a patient, adapted for connection with an external device, the implant comprising:
a. an interface for receiving, by the patient, input to the implant, resulting in input authentication data,
b. a receiver for receiving authentication data from the external device, the authentication data relating to a generated sensation of a sensation generator of the implant or the external device,
c. a computing unit, wherein the computer program product is configured to cause the computing unit to:
   i. analyze the authentication data and the input authentication data, and
   ii. perform authentication of the connection based on the analysis.

According to some embodiments of the fifth part of aspect 248SE the computer program product is configured to cause the implant to accept further communication with further data received by the implant following positive authentication.

According to some embodiments of the fifth part of aspect 248SE the authentication data comprises a timestamp and wherein the input authentication data comprises a timestamp of the input from the patient.

According to some embodiments of the fifth part of aspect 248SE authenticating the connection comprises: calculating a time difference between the timestamp of the authentication data and the timestamp of the input from the patient, and upon determining that the time difference is less than a threshold, authenticating the connection.

According to some embodiments of the fifth part of aspect 248SE the authentication data comprises a number of times that the sensation is generated by the sensation generator, and wherein the input authentication data comprises an input from the patient relating to a number of times the patient detected the sensation, wherein authenticating the connection comprises: upon determining that the number of times of the authentication data and the input authentication data are equal, authenticating the connection.

According to some embodiments of the fifth part of aspect 248SE the further communication between the implant and the external device is a wireless communication.

According to some embodiments of the fifth part of aspect 248SE the communication between the implant and the external device is a wireless communication.

According to some embodiments of the fifth part of aspect 248SE the communication between the implant and the external device is a conductive communication.

According to some embodiments of the fifth part of aspect 248SE the further communication between the implant and the external device is a wireless communication.

The implant according to the second part of aspect 248SE and/or with ability to use any of the methods of the first part of aspect 248SE, and/or with ability to perform the authentication process in any of third part of aspect 248SE and/or with ability to use any of the computer program products of the fourth part of aspect 248SE, may comprise an internal control unit adapted to be involved in at least a part of the actions performed by the implant in at least a part of any one of the embodiments of the above.

### Aspect 249SE Prior Verified Communication—Verifying Authenticity of Instructions Sent from the External Device to the Implant—Embodiments of Aspect 249SE of the Disclosure

In a first part of aspect 249SE, a method of communicating instructions from an external device to an implant implanted in a patient is provided. The method comprising establishing a connection between the external device and the implant, combining a first set of instructions with a previously transmitted set of instructions, forming a first combined set of instructions, transmitting the first combined set of instructions to the implant.

The method further comprising, at the implant, verifying the authenticity of the first combined set of instructions, by: extracting the previously transmitted set of instructions from the first combined set of instructions, comparing the extracted previously transmitted set of instructions with previously received instructions stored in the implant, and upon determining that the extracted previously transmitted set of instructions equals the previously received instructions stored in the implant, running the first set of instructions at the implant and storing the first combined set of instructions in the implant, to be used for verifying a subsequent received set of instructions.

According to some embodiments of the first part of aspect 249SE, step of verifying the authenticity of the first combined set of instructions further comprises upon determining that the extracted previously transmitted set of instructions differs from the previously received instructions stored in the implant, providing feedback related to an unauthorized attempt to instruct the implant.

According to some embodiments of the first part of aspect 249SE, the step of comparing the extracted previously transmitted set of instructions with previously received instructions stored in the implant comprises calculating a difference between the extracted previously transmitted set of instructions with the previously received instructions stored in the implant, and comparing the difference with a threshold value, wherein the extracted previously transmitted set of instructions is determined to equal the previously received instructions stored in the implant in the case of the difference value not exceeding the threshold value.

According to some embodiments of the first part of aspect 249SE, the combined set of instructions comprises the first set of instructions and a cryptographic hash of the previously transmitted set of instructions, wherein the method further comprises, at the implant, calculating a cryptographic hash of the previously received instructions stored in the implant and comparing the calculated cryptographic hash to the cryptographic hash included in the first combined set of instructions.

According to some embodiments of the first part of aspect 249SE, the method further comprises the steps of combining a second set of instructions with the first combined set of instructions, forming a second combined set of instructions, wherein the second combined set of instructions comprises a cryptographic hash of the first combined set of instructions, and transmitting the second combined set of instructions to the implant. The authenticity of the second combined set of instructions is verified at the implant by calculating a cryptographic hash of the first combined set of instructions stored in the implant, and comparing the calculated cryptographic hash with the cryptographic hash included in the received second combined set of instructions. Upon determining that the calculated cryptographic hash of the first combined set of instructions equals the cryptographic hash included in the received second combined set, the second set of instructions is run at the implant, and the second combined set of instruction is stored in the implant, to be used for verifying a subsequent received set of instructions.

According to some embodiments of the first part of aspect 249SE, wherein the first combined set of instructions is transmitted to the implant using a proprietary network protocol.

According to some embodiments of the first part of aspect 249SE, the first combined set of instructions is transmitted to the implant using a standard network protocol.

In a second part of aspect 249SE, a method of communicating instructions from an external device to an implant implanted in a patient is provided. The method comprises the steps of establishing a connection between the external device and the implant, confirming the connection between the implant and the external device, receiving a set of instructions from the external device, as a result of the confirmation, verifying the authenticity of the set of instructions and storing the set of instructions in the implant to be used for verifying authenticity of a subsequently received set of instructions, and transmitting further sets of instructions from the external device to the implant according any embodiment the first part of aspect 249SE.

According to some embodiments of the second part of aspect 249SE, the step of confirming the connection between the implant and the external device comprises: measuring a parameter of the patient, by implant, measuring a parameter of the patient, by external device, comparing the parameter measured by the implant to the parameter measured by the external device, and performing authentication of the connection based on the comparison.

According to some embodiments of the second part of aspect 249SE, the step of confirming the connection between the implant and the external device comprises: generating, by a sensation generator, a sensation detectable by a sense of the patient, by the implant, authentication data, related to the generated sensation, providing, by the patient, input to the external device, resulting in input authentication data, and authenticating the connection based on a comparison of the input authentication data and the authentication data.

In a third part of aspect 249SE, a method of communicating instructions from an external device to an implant implanted in a patient is provided. The method comprising: placing a conductive member, configured to be in connection with the external device, in electrical connection with a skin of the patient for conductive communication with the implant, transmitting, via the electrical connection using conductive communication, a set of instructions from the external device, receiving, at the implant the set of instructions from the external device, storing the set of instructions in the implant to be used for verifying authenticity of a subsequently received set of instructions, and transmitting further sets of instructions from the external device to the implant according any embodiment the first part of aspect 249SE.

According to some embodiments of the third part of aspect 249SE, the method further comprising: a. prior to transmitting, via the electrical connection using conductive communication, a set of instructions from the external device, receiving of an authentication input from a user by a verification unit of the external device, and authenticating the conductive communication between the implant and the external device using the authentication input, as a result of the authentication, transmitting, via the electrical connection using conductive communication, the set of instructions from the external device.

In a fourth part of aspect 249SE, a method of communicating instructions from an external device to an implant implanted in a patient is provided. The method comprises the steps of: receiving, at the implant a set of instructions from a second external device, storing the set of instructions in the implant to be used for verifying authenticity of a subsequently received set of instructions from the external device, and transmitting further sets of instructions from the external device to the implant according to any embodiment the first part of aspect 249SE.

According to some embodiments of the fourth part of aspect 249SE, the second external device transmits the set of instructions using a proprietary network protocol.

According to some embodiments of the fourth part of aspect 249SE, the set of instructions received by the implant from the second external device is encrypted, wherein the method further comprising decrypting the set of instructions and storing the decrypted set of instructions in the implant to be used for verifying authenticity of a subsequently received set of instructions from the external device.

According to some embodiments of either one of the first to fourth part of aspect 249SE, the implant comprises a reset switch, wherein the method further comprising comprises the steps of: activating said reset switch and deleting previously received instructions stored in the implant.

According to some embodiments of either one of the first to fourth part of aspect 249SE, the method further comprises: storing a set of instructions in the implant to be used for verifying authenticity of a subsequently received set of instructions from the external device using any one of the embodiments of the second to fourth part of aspect 249SE.

In a fifth part of aspect 249SE, an implant comprising a transceiver configured to establish a connection with an external device when the implant is implanted in a patient is provided. The implant further comprising a computing unit configured to verify the authenticity of instructions received at the transceiver by: extracting a previously transmitted set of instructions from a first combined set of instructions received by the transceiver, comparing the extracted previously transmitted set of instructions with previously received instructions stored in the implant, upon determining that the extracted previously transmitted set of instructions equals the previously received instructions stored in the implant, running the first set of instructions at the implant.

According to some embodiments of the fifth part of aspect 249SE, the computing unit is configured to: upon determining that the extracted previously transmitted set of instructions differs from the previously received instructions stored in the implant, provide feedback, via a feedback unit of the implant, related to an unauthorized attempt to instruct the implant.

According to some embodiments of the fifth part of aspect 249SE, computing unit is configured to compare the extracted previously transmitted set of instructions with previously received instructions stored in the implant by calculating a difference between the extracted previously transmitted set of instructions with previously received instructions stored in the implant, and compare the difference with a threshold value, wherein the extracted previously transmitted set of instructions is determined to equal the previously received instructions stored in the implant in the case of the difference value not exceeding the threshold value.

According to some embodiments of the fifth part of aspect 249SE, the first combined set of instructions comprises the first set of instructions and a cryptographic hash of the previously transmitted set of instructions, wherein the computing unit is configured to calculate a cryptographic hash of previously received instructions stored in the implant and compare the calculated cryptographic hash to the cryptographic hash included in the first combined set of instructions.

According to some embodiments of the fifth part of aspect 249SE, the computing unit is further configured to verify the authenticity of a second combined set of instructions, the second combined set of instructions comprising a cryptographic hash of the first combined set of instructions, the second combined set of instructions received at the transceiver by: extracting the first combined set of instructions from the second combined set of instructions, calculating a cryptographic hash of the first combined set of instructions and comparing the calculated cryptographic hash with the cryptographic hash included in the received second combined set of instructions, calculating a cryptographic hash of previously received instructions stored in the implant and comparing this to the cryptographic hash included in the extracted first combined set of instructions. Upon determining, by the computing unit, that the cryptographic hash of the first combined set of instructions equals the cryptographic hash included in the received second combined set, and that the cryptographic hash of previously received instructions stored in the implant equals the cryptographic hash included in the extracted first combined set of instructions, the second set of instructions is run at the implant.

According to some embodiments of the fifth part of aspect 249SE, the first combined set of instructions is received at the implant using a proprietary network protocol.

According to some embodiments of the fifth part of aspect 249SE, the first combined set of instructions is received at the implant using a standard network protocol.

In a sixth part of aspect 249SE, an implant comprising a transceiver configured to establish a connection with an external device when the implant is implanted in a patient is provided. The implant further comprising a computing unit configured to verify the authenticity of instructions received at the transceiver by:
a. establishing a connection with the external device,
b. confirming the connection,
c. receiving a set of instructions from the external device,
d. as a result of the confirmation, verifying the authenticity of the set of instructions and storing the set of instructions in the implant to be used for verifying authenticity of a subsequently received set of instructions. The computing unit is further configured to verify the authenticity of further sets of instructions received by the transceiver according to any embodiment of the fifth part of aspect 249SE.

According to some embodiments of the sixth part of aspect 249SE, the computing unit is configured to confirm the connection by: receiving a measured parameter of the patient, the parameter measured by a sensor connected to the implant, receiving a measured parameter of the patient from the external device, comparing the parameter measured by the implant to the parameter measured by the external device, and performing authentication of the connection based on the comparison.

In a seventh part of aspect 249SE, an implant comprising a transceiver configured to establish a connection with an external device when the implant is implanted in a patient is provided. The implant further comprising a computing unit configured to verify the authenticity of instructions received at the transceiver by: receiving, via an electrical connection using conductive communication from the external device, a set of instructions from the external device, storing the set of instructions in the implant to be used for verifying authenticity of a subsequently received set of instructions, wherein the computing unit is configured to verify the authenticity of further sets of instructions received by the transceiver according to any embodiment of the fifth part of aspect 249SE.

In an eight part of aspect 249SE, an implant comprising a transceiver configured to establish a connection with an external device, and a connection with a second external device, when the implant is implanted in a patient is provided. The implant further comprising a computing unit configured to verify the authenticity of instructions received at the transceiver from the external device by: receiving, at the implant a set of instructions from the second external device, storing the set of instructions in the implant to be used for verifying authenticity of a subsequently received set of instructions from the external device, wherein the computing unit is configured to verify the authenticity of further sets of instructions received by the transceiver according to any embodiment of the fifth part of aspect 249SE.

According to some embodiments of the eight part of aspect 249SE, the transceiver is configured to receive the set of instructions from the second external device using a proprietary network protocol.

According to some embodiments of the eight part of aspect 249SE, the set of instructions received by the implant from the second external device is encrypted, wherein the computing unit is configured to decrypt the set of instructions and store the decrypted set of instructions in the implant to be used for verifying authenticity of a subsequently received set of instructions from the external device.

According to some embodiments of any one of the fifth to eight part of aspect 249SE, the implant further comprising a reset switch, wherein the reset switch is configured to delete previously received instructions stored in the implant when being activated.

According to some embodiments of any one of the fifth to eight part of aspect 249SE, the reset switch is further configured to extract factory settings stored in the implant when being activated, wherein the factory settings comprises data to be used for verifying authenticity of a subsequently received set of instructions from the external device, wherein said activation of the reset switch causes said data to be stored in the implant as a set of instructions to be used for verifying authenticity of a subsequently received set of instructions from the external device.

In a ninth part of aspect 249SE, there is provided a system comprising an implant according to embodiments of any one of the fifth part to the eight part of aspect 249SE and an external device. The external device comprises a computing unit configured for: combining a first set of instructions with a previously transmitted set of instructions, forming a combined set of instructions, and transmitting the combined set of instructions to the implant.

In a tenth part of aspect 249SE, there is provided a system comprising an implant according to embodiments of the eight part of aspect 249SE, an external device and a second external device. The external device is configured to receive a set of instructions from the second external device, store said set of instructions, wherein the external device comprises a computing unit configured to combining a first set of instructions with a said stored set of instructions, forming a combined set of instructions, transmitting the combined set of instructions to the implant.

In an eleventh part of aspect 249SE, there is provided a computer program product comprising a computer-readable storage medium with instructions adapted to carry out at least parts of embodiments of any one of the first to third part of aspect 249SE when executed by a device having processing capability.

In a twelfth part of aspect 249SE, there is provided a computer program product configured to be used by the implant of any of embodiment of the first to fourth parts of aspect 249SE, when executed by the implant or external device having processing capability.

In a thirteenth part of aspect 249SE, there is provided a computer program product comprising a computer-readable storage medium with instructions adapted to carry out at least parts of any of embodiments of the fifth to eighth parts of aspect 249SE when executed by the implant or external device having processing capability

According to some embodiments the implant of any one of the fifth to eighth parts of aspect 249SE, and/or with ability to use any of the methods of the first to fourth parts of aspect 249SE, and/or of the system of the ninth or tenth parts of aspect 249SE, and/or able to use a computer program product of the eleventh to thirteenth parts of aspect 249SE, wherein the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments the implant of any one of the fifth to eighth parts of aspect 249SE, and/or with ability to use any of the methods of the first to fourth parts of aspect 249SE, and/or of the system of the ninth or tenth parts of aspect 249SE, and/or able to use a computer program product of the eleventh to thirteenth parts of aspect 249SE comprises an internal control unit adapted to be involved in at least a part of the actions performed by the implant in at least a part of any one of the other embodiments of aspect 249SE.

In a fifteenth part of aspect 249SE there is provided a system comprising an implant of the fifth to eighth parts of the sixth aspect, an external device and a second external device, wherein the external device is configured to:
a. receive a set of instructions from the second external device,
b. store said set of instructions, wherein the external device comprises a computing unit configured to:
c. combining a first set of instructions with a said stored set of instructions, forming a combined set of instructions,
d. transmitting the combined set of instructions to the implant.

### Aspect 250SE Dual Protocols—Two Wireless Communication Protocols for Communication—Embodiments of Aspect 250SE of the Disclosure

In a first part of aspect 250SE, an external device configured for communication with an implant when implanted in a patient, is provided. The external device is further configured to communication with a second external device. The external device comprising at least one wireless transceiver configured for wireless communication with the second external device and the implant, wherein the wireless transceiver is configured to communicate with the implant using a proprietary network protocol, wherein the at least one wireless transceiver is configured to communicate with the second external device using a standard network protocol.

According to some embodiments of the first part of aspect 250SE, the at least one wireless transceiver comprises a first wireless transceiver configured for communicating with the second external device, and a second wireless transceiver) configured for communicating with the implant.

According to some embodiments of the first part of aspect 250SE, the external device further comprising a computing unit adapted for configuring the at least one wireless transceiver to communicate with the implant using the proprietary network protocol and adapted for configuring the at least one wireless transceiver to communicate with the second external device using the standard network protocol.

According to some embodiments of the first part of aspect 250SE, the standard network protocol is one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments of the first part of aspect 250SE, a communication range of the proprietary network protocol is less than a communication range of the standard network protocol.

According to some embodiments of the first part of aspect 250SE, a frequency band of the proprietary network protocol differs from a frequency band of the standard network protocol.

According to some embodiments of the first part of aspect 250SE, the frequency band of the proprietary network protocol is 13.56 MHz, wherein the standard network protocol in one from the list of
WLAN type protocol;
Bluetooth type protocol.
BLE type protocol
3G/4G/5G type protocol
GSM type protocol.

According to some embodiments of the first part of aspect 250SE, wherein the external device comprises: a sensor for measuring a parameter of the patient, by the external device, and an external computing unit configured for:
i. receiving a parameter of the patient, from the implant,
ii. comparing the parameter measured by the external device to the parameter measured by the implant, and
iii. performing authentication of a wireless connection with the implant based on the comparison.

According to some embodiments of the first part of aspect 250SE, the sensor is configured to measure a pulse of the patient.

According to some embodiments of the first part of aspect 250SE, the sensor is configured to measure a respiration rate of the patient.

According to some embodiments of the first part of aspect 250SE, the sensor is configured to measure a temperature of the patient.

According to some embodiments of the first part of aspect 250SE, the sensor is configured to measure at least one sound of the patient.

According to some embodiments of the first part of aspect 250SE, the sensor is configured to measure at least one physical movement of the patient.

According to some embodiments of the first part of aspect 250SE, the measured parameter, by the external device is provided with a timestamp and the measured parameter received from the implant is provided with a timestamp, wherein the comparison of the parameter measured at the implant to the parameter measured by the external device comprises comparing the timestamp of the measured parameter received from the implant to the timestamp of the measured parameter by the external device.

According to some embodiments of the first part of aspect 250SE, the external computing unit is configured to calculate a difference value between the parameter measured by the implant and the parameter measured by the external device, and wherein the external computing unit is further configured to authenticate the wireless connection if the difference value is less than a predetermined threshold difference value, and to not authenticate the wireless connection if the difference value equals or exceeds the predetermined threshold difference value.

According to some embodiments of the first part of aspect 250SE, the external device comprises a clock, configured for synchronization with a clock of the implant.

According to some embodiments of the first part of aspect 250SE, external device comprising an interface for receiving, by the patient, input to the external device, resulting in input authentication data, and a receiver for receiving authentication data from the implant, the authentication data relating to a generated sensation of a sensation generator connected to the implant. The external computing unit is configured for:
i. comparing the authentication data to the input authentication data, and
ii. performing authentication of the connection based on the comparison.

According to some embodiments of the first part of aspect 250SE, the external device is one from the list of: a wearable external device, and a handset.

According to some embodiments of the first part of aspect 250SE, the external device is configured to be placed in electrical connection with a conductive member, for conductive communication with the implant.

In a second part of aspect 250SE, a method for communicating with an implant when implanted in a patient, and with a second external device, is provided. The method comprising: establishing wireless communication between at least one wireless transceiver of an external device and a second external device and the implant, wherein the communication between the external device and the implant uses a proprietary network protocol, and wherein the wireless communication between the external device and the second external device uses a standard network protocol.

According to some embodiments of the second part of aspect 250SE, the wireless communication between the external device and the second external device is performed by a first wireless transceiver of the at least one wireless transceiver and, wherein the wireless communication between the external device and the implant is performed by a second wireless transceiver of the at least one wireless transceiver.

According to some embodiments of the second part of aspect 250SE, the method further comprising the step of configuring, by a computing unit of the external device, the at least one wireless transceiver to communicate between the external device and the implant using a proprietary network protocol, and to communicate between the external device and the second external device using a standard network protocol.

According to some embodiments of the second part of aspect 250SE, the standard network protocol is one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments of the second part of aspect 250SE, a communication range of the proprietary network protocol is less than a communication range of the standard network protocol.

According to some embodiments of the second part of aspect 250SE, a frequency band of the proprietary network protocol differs from a frequency band of the standard network protocol.

According to some embodiments of the second part of aspect 250SE, the frequency band of the proprietary network protocol is 13.56 MHz, wherein the standard network protocol in one from the list of
WLAN type protocol;
Bluetooth type protocol
BLE type protocol
3G/4G/5G type protocol
GSM type protocol.

According to some embodiments of the second part of aspect 250SE, the wireless communication between the external device and the implant is authenticated by the steps of:
i. measuring a parameter of the patient, by the external device
ii. receiving a parameter of the patient, from the implant,
iii. comparing the parameter measured by the external device to the parameter measured by the implant, and
iv. performing authentication of a wireless connection based on the comparison.

According to some embodiments of the second part of aspect 250SE, the parameter of the patient is a pulse of the patient.

According to some embodiments of the second part of aspect 250SE, the parameter of the patient is a respiration rate of the patient.

According to some embodiments of the second part of aspect 250SE, the parameter of the patient is a temperature of the patient.

According to some embodiments of the second part of aspect 250SE, the parameter of the patient is at least one sound of the patient.

According to some embodiments of the second part of aspect 250SE, the parameter of the patient is at least one physical movement of the patient.

According to some embodiments of the second part of aspect 250SE, the measured parameter, by the external device is provided with a timestamp and the measured parameter received from the implant is provided with a timestamp, wherein the comparison of the parameter measured at the implant to the parameter measured by the external device comprises comparing the timestamp of the measured parameter received from the implant to the timestamp of the measured parameter by the external device.

According to some embodiments of the second part of aspect 250SE, the step of comparing the parameter measured by the implant to the parameter measured by the external device comprises calculating a difference value between the parameter measured by the implant and the parameter measured by the external device, wherein the step of performing authentication comprises: authenticating the wireless connection if the difference value is less than a predetermined threshold difference value, and not authenticating the wireless connection if the difference value equals or exceeds the predetermined threshold difference value.

According to some embodiments of the second part of aspect 250SE, the method further comprises synchronization of a clock of the external device with a clock of the implant.

According to some embodiments of the second part of aspect 250SE, method comprising placing a conductive member, configured to be in connection with the external device, in electrical connection with a skin of the patient for conductive communication with the implant.

According to some embodiments of the second part of aspect 250SE, the wireless communication between the external device and the implant is authenticated by the steps of:
generating, by a sensation generator, a sensation detectable by a sense of the patient,
storing, by the implant, authentication data, related to the generated sensation,
providing, by the patient, input to the external device, resulting in input authentication data,
authenticating the wireless communication based on a comparison of the input authentication data and the authentication data.

According to some embodiments of the second part of aspect 250SE, the method comprising the step of communicating data between the implant and the external device using the proprietary network protocol following positive authentication.

According to some embodiments of the second part of aspect 250SE, the method comprising establishing communication between the second externa device and a third external device, wherein the communication between the second externa device and the third external device is authenticated using a verification process at the third external device.

According to some embodiments of the second part of aspect 250SE, the third external device is operated by a caretaker of the patient.

According to some embodiments of the second part of aspect 250SE, the method comprising the step of authenticating the wireless communication between the external device and the second external device using a verification process at the second external device, wherein communication between the external device and the second external device requires the communication to be authenticated.

According to some embodiments of the second part of aspect 250SE, the second external device is operated by a caretaker of the patient.

In a third part of aspect 250SE, a computer-readable storage medium is provided. The computer-readable storage medium comprises instructions adapted to carry out the method of any embodiment of the second part of aspect 250SE when executed by a device having processing capability.

In a fourth part of aspect 250SE, a system comprising an external device according to any embodiment of the first part of aspect 250SE, and an implant implanted in the patient, is provided. The implant comprises a wireless receiver configured for receiving communication using the proprietary network protocol.

According to some embodiments of the fourth part of aspect 250SE, the wireless receiver of the implant is configured for only receiving communication using the proprietary network protocol.

According to some embodiments of the fourth part of aspect 250SE, an antenna of the wireless receiver of the implant is configured to only receive in a first frequency band, wherein the frequency band of the proprietary network protocol is included in the first frequency band.

According to some embodiments of the fourth part of aspect 250SE, the frequency band of the standard network protocol is not included in the first frequency band.

According to some embodiments of the fourth part of aspect 250SE, the implant comprises a computing unit configured to only altering an operation of the implant based on data received using the proprietary network protocol.

According to some embodiments of the fourth part of aspect 250SE, the system further comprising a second external device.

According to some embodiments of the fourth part of aspect 250SE, the second external device comprises an interface for authentication of the communication with external device, wherein communication between the external device and the second external device requires the communication to be authenticated.

According to some embodiments of the fourth part of aspect 250SE, the system further comprising a third external device configured to communicate with the second external device.

According to some embodiments of the fourth part of aspect 250SE, the third external device comprises an interface for authentication of the communication with the second external device, wherein communication between the third external device and the second external device requires the communication to be authenticated.

According to some embodiments of the fourth part of aspect 250SE, the third external device comprises an interface for authentication of the communication with the second external device, wherein communication between the third external device and the second external device requires the communication to be authenticated.

According to some embodiments of the fourth part of aspect 250SE, the third external device is operated by a caretaker of the patient.

According to some embodiments of the first part of aspect 250SE the external device is configured to communicate further data via the conductive communication with the implant.

In a fifth part of aspect 250SE, there is provided a computer program product of, or adapted to run on, an external device configured for communication with an implant when implanted in a patient, and with a second external device, the external device comprising
at least one wireless transceiver configured for wireless communication with the second external device and the implant, wherein the computer program product is configured to cause the at least one wireless transceiver to communicate with the implant using a proprietary network protocol,
wherein the computer program product is configured to cause the at least one wireless transceiver to communicate with the second external device using a standard network protocol.

According to some embodiments of the fifth part of aspect 250SE the at least one wireless transceiver comprises a first wireless transceiver and a second wireless transceiver, wherein the computer program product is configured to cause the first wireless transceiver to communicate with the second external device, and wherein the computer program product is configured to cause the second wireless transceiver to communicate with the implant.

According to some embodiments of the fifth part of aspect 250SE, The external device comprises a computing unit adapted for configuring the computer program product to cause the at least one wireless transceiver to communicate with the implant using the proprietary network protocol and adapted for configuring the computer program product to cause the at least one wireless transceiver to communicate with the second external device using the standard network protocol.

According to some embodiments of the fifth part of aspect 250SE the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.

According to some embodiments of the fifth part of aspect 250SEa communication range of the proprietary network protocol is less than a communication range of the standard network protocol.

According to some embodiments of the fifth part of aspect 250SEa frequency band of the proprietary network protocol differs from a frequency band of the standard network protocol.

According to some embodiments of the fifth part of aspect 250SE the frequency band of the proprietary network protocol is 13.56 MHz, wherein the standard network protocol in one from the list of:
WLAN type protocol,
Bluetooth type protocol,
BLE type protocol,
3G/4G/5G type protocol, and
GSM type protocol.

According to some embodiments of the fifth part of aspect 250SE the external device comprises:
a sensor for measuring a parameter of the patient, by the external device, and
an external computing unit, wherein the computer program product is configured to cause the external computing unit to:
i. receive a parameter of the patient, from the implant,
ii. compare the parameter measured by the external device to the parameter measured by the implant, and
iii. perform authentication of a wireless connection with the implant based on the comparison,

According to some embodiments of the fifth part of aspect 250SE the computer program product is configured to cause the sensor to measure a pulse of the patient.

According to some embodiments of the fifth part of aspect 250SE the computer program product is configured to cause the sensor to measure a respiration rate of the patient.

According to some embodiments of the fifth part of aspect 250SE the computer program product is configured to cause the sensor to measure a temperature of the patient.

According to some embodiments of the fifth part of aspect 250SE the computer program product is configured to cause the sensor to measure at least one sound of the patient.

According to some embodiments of the fifth part of aspect 250SE the computer program product is configured to cause the sensor to measure at least one physical movement of the patient.

According to some embodiments of the fifth part of aspect 250SE the measured parameter, by the external device is provided with a timestamp and the measured parameter received from the implant is provided with a timestamp, wherein the comparison of the parameter measured at the implant to the parameter measured by the external device comprises comparing the timestamp of the measured parameter received from the implant to the timestamp of the measured parameter by the external device.

According to some embodiments of the fifth part of aspect 250SE the computer program product is configured to cause the external computing unit to calculate a difference value between the parameter measured by the implant and the parameter measured by the external device, and wherein the computer program product is configured to cause the external computing unit to authenticate the wireless connection if the difference value is less than a predetermined threshold difference value, and to not authenticate the wireless connection if the difference value equals or exceeds the predetermined threshold difference value.

According to some embodiments of the fifth part of aspect 250SE the computer program product is configured to cause a clock of the external device, to be synchronized with a clock of the implant.

According to some embodiments of the fifth part of aspect 250SE the external device comprises:
an interface for receiving, by the patient, input to the external device, resulting in input authentication data,
a receiver for receiving authentication data from the implant, the authentication data relating to a generated sensation of a sensation generator connected to the implant, and
an external computing unit, wherein the computer program product is configured to cause the external computing unit to:
i. compare the authentication data to the input authentication data, and
ii. perform authentication of the connection based on the comparison.

According to some embodiments of the fifth part of aspect 250SE the external device is configured to be placed in electrical connection with a conductive member, for conductive communication with the implant, and wherein the computer program product is configured to cause the external device to communicate further data via the conductive communication with the implant.

According to some embodiments, the implant of the system of the fourth part of aspect 250SE, and/or with ability to communicate with the external device of the first part of aspect 250SE, and/or with ability to use any of the methods of the second part of aspect 250SE, and/or with ability to use a computer program product of the third or fifth parts of aspect 250SE, wherein the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments, the implant of the system of the fourth part of aspect 250SE, and/or with ability to communicate with the external device of the first part of aspect 250SE, and/or with ability to use any of the methods of the second part of aspect 250SE, and/or with ability to use a computer program product of the third or fifth parts of aspect 250SE, comprises an internal control unit adapted to be involved in at least a part of the actions performed by the implant in at least a part of any one of the embodiments of aspect 250SE.

### Aspect 251SE 2-Part Key NFC—Two Communication Methods for Sending Encryption Keys—Embodiments of Aspect 251SE of the Disclosure

In a first part of aspect 251SE, a method for encrypted communication between an external device and an implant implanted in a patient is provided. The external device is adapted to communicate with the implant using two separate communication methods. A communication range of a first communication method is less than a communication range of a second communication method. The method for encrypted communication comprises sending a first part of a key from the external device to the implant, using the first communication method. The method for encrypted communication comprises sending a second part of the key from the external device to the implant, using the second communication method. The method for encrypted communication comprises sending encrypted data from the external device to the implant using the second communication method. The method for encrypted communication comprises deriving, in the implant a combined key from the first part of the key and second part of the key. The method for encrypted communication comprises decrypting the encrypted data, in the implant, using the combined key.

According to some embodiments of the first part of aspect 251SE the first communication method comprises RFID, Bluetooth, BLE, NFC, NFC-V, Infrared based communication, or Ultrasound based communication.

According to some embodiments of the first part of aspect 251SE the communication range of the first communication method is less than 10 meters.

According to some embodiments of the first part of aspect 251SE the communication range of the first communication method is less than 2 meters.

According to some embodiments of the first part of aspect 251SE a center frequency of a frequency band of the first communication method is 13.56 MHz or 27.12 MHz

According to some embodiments of the first part of aspect 251SE the implant comprises a passive receiver for receiving the first part of the key.

According to some embodiments of the first part of aspect 251SE the passive receiver of the implant comprises a loop antenna.

According to some embodiments of the first part of aspect 251SE the method comprises limiting the communication range of the first communication method by adjusting the frequency and/or phase of the transmitted information.

According to some embodiments of the first part of aspect 251SE the method further comprises wirelessly receiving, at the implant, a third part of the key from a second external device. The combined key is now derived from the first part of the key, the second part of the key and the third part of the key.

According to some embodiments of the first part of aspect 251SE the external device is adapted to be in electrical connection with the implant, using the body as a conductor. The method may then further comprise confirming the electrical connection between the implant and the external device and, as a result of the confirmation, decrypting the encrypted data in the implant and using the decrypted data for instructing the implant.

According to some embodiments of the first part of aspect 251SE the second communication method comprises WLAN, Bluetooth, BLE, 3G/4G/5G, or GSM.

According to some embodiments of the first part of aspect 251SE the encrypted data comprises instructions for updating a control program running in the implant, wherein the implant comprises a computing unit configured to update a control program running in the implant using the decrypted data.

According to some embodiments of the first part of aspect 251SE the encrypted data comprises instructions for operating the implant, wherein the implant comprises a computing unit configured to operate the implant using the decrypted data.

According to some embodiments of the first part of aspect 251SE the method may further comprise the steps of Generating, by a sensation generator, a sensation detectable by a sense of the patient.

Storing, by the implant, authentication data, related to the generated sensation.

Providing, by the patient, input to the external device, resulting in input authentication data.

Authenticating the first or second communication method based on a comparison of the input authentication data and the authentication data.

As a result of positive authentication of the first or second communication method, decrypting the encrypted data in the implant and using the decrypted data for instructing the implant.

According to some embodiments of the first part of aspect 251SE the method may further comprise the step of transmitting the input authentication data from the external device to the implant, wherein the comparison is performed by the implant.

According to some embodiments of the first part of aspect 251SE the method may further comprise the steps of:

Measuring a parameter of the patient, by the implant.

Measuring the parameter of the patient, by the external device.

Comparing the parameter measured by the implant to the parameter measured by the external device.

Authenticating the first or second communication method based on the comparison.

As a result of positive authentication of the first or second communication method, decrypting the encrypted data in the implant and using the decrypted data for instructing the implant.

According to some embodiments of the first part of aspect 251SE the method may further comprise the step of transmitting the parameter measured by the external device from the external device to the implant, wherein the comparison is performed by the implant.

According to some embodiments of the first part of aspect 251SE the method further comprises placing a conductive member, configured to be in connection with the external device, in electrical connection with a skin of the patient for conductive communication with the implant.

According to some embodiments of the first part of aspect 251SE the communication is cancelled or amplified for at least one point by destructive or constructive interference respectively.

According to some embodiments of the first part of aspect 251SE the communication has a wavelength, λ and the method comprises transmitting the communication from a first point located at a distance, D, away from the at least one point. The communication may be cancelled for the at least one point by transmitting the communication from a second point located at a distance D±½Zλ from the at least one point, wherein Z is any integer, or alternatively, the communication may be amplified for the at least one point by transmitting the communication from a second point located at a distance D±Zλ from the at least one point.

According to some embodiments of the first part of aspect 251SE the method further comprises transmitting the communication from a first point with a phase, P. The communication may be cancelled for the at least one point by transmitting the communication from a second point with a phase P±Zπ, wherein Z is any integer, or alternatively, the communication may be amplified for the at least one point by transmitting the communication from the second point with a phase P±2Zπ. For this, a distance between the first point and the at least one point equals the distance between the second point and the at least one point plus or minus any integer times a wavelength, λ, of the communication.

According to some embodiments of the first part of aspect 251SE the first point is a first transmitter and the second point is a second transmitter.

According to some embodiments of the first part of aspect 251SE the first point and the second point may be moved with respect to each other such that the at least one point is spatially shifted.

According to some embodiments of the first part of aspect 251SE the first point is associated with the implant and the second point is associated with the external device.

According to some embodiments of the first part of aspect 251SE the first point is a first slit and the second point is a second slit. The first and second slits may be adapted to receive the same communication from a single transmitter.

According to some embodiments of the first part of aspect 251SE a phase, P, of the communication is alternated as to spatially shift the at least one point.

According to some embodiments of the first part of aspect 251SE the method further comprises the steps of Transmitting the communication by the external device via the first and second points.

Measuring by the implant the interference for at least two points.

Comparing the measured interference with reference data pertaining to an authorized external device.

Authenticating the communication based on the results from comparing the measured interference with the reference data.

In a second part of aspect 251SE, there is provided an external device configured for encrypted communication with an implant implanted in a patient. The external device comprises a first and a second wireless transceiver for communication with the implant using two separate communication methods. A communication range of a first communication method is less than a communication range of the second communication method. The first wireless transceiver is configured to send a first part of a key to the implant, using the first communication method. The second wireless transceiver is configured to send a second part of a key to the implant, using the second communication method. The second wireless transceiver is further configured to send encrypted data the implant using the second communication method. The encrypted data is configured to be decrypted by a combined key derived from the first part of the key and second part of the key.

According to some embodiments of the second part of aspect 251SE the first communication method implemented by the first wireless transceiver comprises RFID, Bluetooth, BLE, NFC, NFC-V, Infrared based communication, or Ultrasound based communication.

According to some embodiments of the second part of aspect 251SE the communication range of the first communication method is less than 10 meters.

According to some embodiments of the second part of aspect 251SE the communication range of the first communication method is less than 2 meters.

According to some embodiments of the second part of aspect 251SE a center frequency of a frequency band of the first communication method is 13.56 MHz or 27.12 MHz.

According to some embodiments of the second part of aspect 251SE the first wireless transceiver comprises a loop antenna for transmission of data using the first communication method.

According to some embodiments of the second part of aspect 251SE the first wireless transceiver is configured to limit the communication range of the first communication method by adjusting a frequency and/or a phase of the communication.

According to some embodiments of the second part of aspect 251SE the second communication method implemented by the second wireless transceiver comprises WLAN, Bluetooth, BLE, 3G/4G/5G, or GSM.

According to some embodiments of the second part of aspect 251SE the encrypted data comprises instructions for updating a control program running in the implant.

According to some embodiments of the second part of aspect 251SE the encrypted data comprises instructions for operating the implant.

According to some embodiments of the second part of aspect 251SE the communication has a wavelength, λ. The external device transmits the communication from a first point located at a distance, D, away from at least one point. The communication may be cancelled for the at least one point by transmitting the communication from a second point located at a distance D±½Zλ from the at least one point, wherein Z is any integer, or alternatively, the communication may be amplified for the at least one point by transmitting the communication from a second point located at a distance D±Zλ from the at least one point.

According to some embodiments of the second part of aspect 251SE the communication has a phase, P, and wherein the external device transmits the communication from a first point. The communication may be cancelled for at least one point by transmitting the communication from a second point with a phase P±Zπ, wherein Z is any integer, or alternatively, the communication may be amplified for the at least one point by transmitting the communication from a second point with a phase P±2Zπ. For this, a distance between the first point and the at least one point may equal the distance between the second point and the at least one point plus or minus any integer times a wavelength, λ, of the communication.

According to some embodiments of the second part of aspect 251SE the first point is a first transmitter and the second point is a second transmitter.

According to some embodiments of the second part of aspect 251SE the first point and the second point may be moved with respect to each other such that the at least one point is spatially shifted.

According to some embodiments of the second part of aspect 251SE the first point is associated with the implant and the second point is associated with the external device.

According to some embodiments of the second part of aspect 251SE the first point is a first slit and the second point is a second slit. The first and second slits are adapted to receive the same communication from a single transmitter.

According to some embodiments of the second part of aspect 251SE a phase, P, of the communication is alternated as to spatially shift the at least one point.

In a third part of aspect 251SE, there is provided an implant configured for encrypted communication with an external device, when implanted in a patient. The implant comprises a first and a second wireless receiver for communication with the external device using two separate communication methods. A communication range of a first communication method is less than a communication range of the second communication method. The first wireless receiver is configured to receive a first part of a key from the external device, using the first communication method. The second wireless receiver is configured to receive a second part of a key from the external device, using the second communication method. The second wireless receiver is further configured to receive encrypted data from the external device using the second communication method. The implant further comprises a computing unit configured to derive a combined key from the first part of the key and the second part of the key, and decrypt the encrypted data using the combined key.

According to some embodiments of the third part of aspect 251SE the first communication method implemented by the first wireless receiver comprises RFID, Bluetooth, BLE, NFC, NFC-V, Infrared based communication, or Ultrasound based communication.

According to some embodiments of the third part of aspect 251SE the communication range of the first communication method is less than 10 meters.

According to some embodiments of the third part of aspect 251SE the communication range of the first communication method is less than 2 meters.

According to some embodiments of the third part of aspect 251SE a center frequency of a frequency band of the first communication method is 13.56 MHz or 27.12 MHz.

According to some embodiments of the third part of aspect 251SE the first wireless receiver is a passive receiver for receiving the first part of the key.

According to some embodiments of the third part of aspect 251SE the passive receiver comprises a loop antenna.

According to some embodiments of the third part of aspect 251SE the implant is configured to wirelessly receive a third part of the key from a second external device. The computing unit may be configured to derive the combined key from the first part of the key, the second part of the key and the third part of the key.

According to some embodiments of the third part of aspect 251SE the implant is in electrical connection with the external device, using the body as a conductor. The implant further comprises an authentication unit configured to confirm the electrical connection between the implant and the external device. The computing unit is configured for, as a result of the confirmation, decrypting the encrypted data and using the decrypted data for instructing the implant.

According to some embodiments of the third part of aspect 251SE the second communication method implemented by the second wireless receiver comprises WLAN, Bluetooth, BLE, 3G/4G/5G, or GSM.

According to some embodiments of the third part of aspect 251SE the encrypted data comprises instructions for updating a control program running in the implant, wherein the computing unit is configured to update a control program running in the implant using the decrypted data.

According to some embodiments of the third part of aspect 251SE the encrypted data comprises instructions for operating the implant, wherein the computing unit is configured to operate the implant using the decrypted data.

According to some embodiments of the third part of aspect 251SE the implant further comprises a first sensor for measuring a parameter of the patient. The computing unit may be further configured for:

Receiving a parameter of the patient, from the external device.

Comparing the parameter measured by the implant to the parameter measured by the external device.

Authenticating the first or second communication method based on the comparison.

As a result of positive authentication of the first or second communication method, decrypting the encrypted data in the implant and using the decrypted data for instructing the implant.

According to some embodiments of the third part of aspect 251SE the implant may be connected to a sensation generator. The implant may be configured for storing authentication data, related to a sensation generated by the sensation generator, and receiving input authentication data from the external device. The implant may further comprise an internal computing unit configured for: Authenticating the first or second communication method based on the comparison.

As a result of positive authentication of the first or second communication method, decrypting the encrypted data in the implant and using the decrypted data for instructing the implant.

According to some embodiments of the third part of aspect 251SE the implant may be further configured for: Receiving the communication from a first and a second point of the external device.

Measuring the interference for at least two points.

Comparing the measured interference with reference data pertaining to an authorized external device.

Authenticating the communication based on the results from comparing the measured interference with the reference data.

In a fourth part of aspect 251SE, there is provided a system comprising an external device of the second part of aspect 251SE in communication with an implant of the third part of aspect 251SE.

According to some embodiments of the fourth part of aspect 251SE the system further comprises a conductive member configured to be in electrical connection with the external device. The conductive member may be configured to be placed in electrical connection with a skin of the patient for conductive communication with the implant.

According to some embodiments of the fourth part of aspect 251SE the system further comprises a second external device. The second external device may be configured for communication with the external device. The external device may be configured for receiving the encrypted data from the second external device and relaying the encrypted data to the implant using the second communication method.

According to some embodiments of the fourth part of aspect 251SE second external device comprises an interface for authentication of the communication with the external device. Communication between the external device and the second external device may require the communication to be authenticated.

According to some embodiments of the fourth part of aspect 251SE the second external device is operated by a healthcare provider of the patient.

According to some embodiments of the first part of aspect 251SE the method further comprises confirming, by the patient, the communication between the external device and the implant.

According to some embodiments of the first part of aspect 251SE the method further comprises sending a third part of the key from the external device to the implant, using a conductive communication method, wherein the combined key is derived from the first part of the key, the second part of the key and the third part of the key.

In a fifth part of aspect 251SE, there is provided a system comprising an external device according to the second part of aspect 251SE, further comprising a conductive member configured to be placed in electrical connection with a skin of a patient for conductive communication with an implant implanted in the patient.

According to some embodiments of the fifth part of aspect 251SE the conductive member is integrally connected to the external device.

According to some embodiments of the fifth part of aspect 251SE the conductive member comprises a wireless communication interface and is communicatively connected to the external device.

According to some embodiments of the third part of aspect 251SE the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

In a sixth part of aspect 251SE, there is provided a computer program product of, or arranged to run on, an external device configured for encrypted communication with an implant implanted in a patient, the external device comprising a first and a second wireless transceiver for communication with the implant using two separate communication methods, wherein a communication range of a first communication method is less than a communication range of the second communication method, wherein the computer program product is configured to cause the first wireless transceiver to send a first part of a key to the implant, using the first communication method, wherein the computer program product is configured to cause the second wireless transceiver to send a second part of a key to the implant, using the second communication method, wherein the computer program product is configured to cause the second wireless transceiver to send encrypted data the implant using the second communication method, wherein the encrypted data is configured to be decrypted by a combined key derived from the first part of the key and the second part of the key.

According to some embodiments of the sixth part of aspect 251SE the first communication method implemented by the first wireless transceiver comprises one from a list of
RFID,
Bluetooth,
BLE,
NFC,
NFC-V,
Infrared based communication, and
Ultrasound based communication.

According to some embodiments of the sixth part of aspect 251SE the communication range of the first communication method is less than 10 meters.

According to some embodiments of the sixth part of aspect 251SE the communication range of the first communication method is less than 2 meters.

According to some embodiments of the sixth part of aspect 251SE a frequency of a frequency band of the first communication method is 13.56 MHz or 27.12 MHz.

According to some embodiments of the sixth part of aspect 251SE the computer program product is configured to cause the first wireless transceiver to limit the communication range of the first communication method by adjusting the frequency and/or phase of the transmitted information.

According to some embodiments of the sixth part of aspect 251SE the second communication method implemented by the second wireless transceiver comprises one from a list of:
WLAN,
Bluetooth,
BLE,
3G/4G/5G, and
GSM.

According to some embodiments of the sixth part of aspect 251SE the encrypted data comprises instructions for updating a control program running in the implant.

According to some embodiments of the sixth part of aspect 251SE the encrypted data comprises instructions for operating the implant.

According to some embodiments of the sixth part of aspect 251SE the communication has a wavelength, λ, and wherein the computer program product is configured to cause the external device to transmit the communication from a first point located at a distance, D, away from at least one point, wherein the communication is cancelled for the at least one point by having the computer program product being configured to cause the external device to:
transmit the communication from a second point located at a distance D±½Zλ from the at least one point, wherein Z is any integer;
or alternatively,
wherein the communication is amplified for the at least one point by having the computer program product being configured to cause the external device to:
   transmit the communication from a second point located at a distance D±Zλ from the at least one point.

According to some embodiments of the sixth part of aspect 251SE the communication has a phase, P, and wherein the computer program product is configured to cause the external device to transmit the communication from a first point, wherein the communication is cancelled for at least one point by having the computer program product being configured to cause the external device to:
transmit the communication from a second point with a phase P±Zπ, wherein Z is any integer;
or alternatively,
wherein the communication is amplified for the at least one point by having the computer program product being configured to cause the external device to:
   transmit the communication from the second point with a phase P±2Zπ,
wherein a distance between the first point and the at least one point equals the distance between the second point and the at least one point plus or minus any integer times a wavelength, λ, of the communication.

According to some embodiments of the sixth part of aspect 251SE the first point is a first transmitter and the second point is a second transmitter.

According to some embodiments of the sixth part of aspect 251SE the computer program product is configured to cause the first point and the second point to be moved with respect to each other such that the at least one point is spatially shifted.

According to some embodiments of the sixth part of aspect 251SE the first point is associated with the implant and wherein the second point is associated with the external device.

According to some embodiments of the sixth part of aspect 251SE the first point is a first slit and the second point is a second slit, the first and second slits being adapted to receive the same communication from a single transmitter.

According to some embodiments of the sixth part of aspect 251SE the computer program product is configured to cause a phase, P, of the communication to be alternated as to spatially shift the at least one point.

According to some embodiments the implant according to at least a part of any one of embodiments of the first, fifth, or sixth parts of aspect 251SE, comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments the implant according to, or presented in, any one of the embodiments of aspect 251SE, comprises an internal control unit adapted to be involved in at least a part of the actions performed by the implant in at least a part of any one of the embodiments of aspect 251SE.

### Aspect 252SE Dual Systems—Two Communication Systems for Communication Between Implant and External Device—Embodiments of Aspect 252SE of the Disclosure

In a first part of aspect 252SE, there is provided a method for communication between an external device and an implant when implanted in a patient, the method comprising: using a first communication system for sending data from the external device to the implant, and using a second, different, communication system for receiving, at the external device, data from the implant.

According to embodiments of the first part of aspect 252SE, the implant comprises a computing unit configured for: receiving, at the implant, a first key from an external device, deriving a combined key using the first key and a second key held by the implant, decrypting the data using the combined key, and using the decrypted data for instructing the implant.

According to embodiments of the first part of aspect 252SE, the method further comprises: receiving, at the implant a third key being generated by a second external device, separate from the external device or by another external device being a generator of the second key on behalf of the second external device, the third key being received at the implant from anyone of, the external device, the second external device, and the generator of the second key, and deriving the combined key using the first and third keys and the second key held by the implant.

According to embodiments of the first part of aspect 252SE, the method further comprises: confirming the connection via the first communication system between the implant and the external device, and as a result of the confirmation, instructing the implant based on data sent from the external device.

According to embodiments of the first part of aspect 252SE, the method further comprises: confirming the connection, via the first communication system, between the implant and the external device, and as a result of the confirmation, instructing the implant based on the decrypted data.

According to embodiments of the first part of aspect 252SE, the method further comprises: altering, by the computing unit, the operation of the implant based on the data decrypted in the implant.

According to embodiments of the first part of aspect 252SE, the first communication system is configured for wireless communication using a first network protocol, wherein the second communication system is configured for wireless communication using a second network protocol.

According to embodiments of the first part of aspect 252SE, the first or second network protocol is a proprietary network protocol, wherein the other network protocol is a standard network protocol.

According to embodiments of the first part of aspect 252SE, the method further comprises: using a third communication system, the third communication system being different than the first and second communication system, for sending data from a second external device, separate from the external device, to the implant.

According to embodiments of the first part of aspect 252SE, the data received at the external device from the implant comprises feedback signals from the implant including one or more from the list of: physiological or physical sensor parameters related to the status of the body of the patient, and physical or functional parameters related to status of the implant.

In a second part of aspect 252SE, there is provided an external device configured for communication with an implant when implanted in a patient, the external device comprising
a first communication system for sending data to the implant, and
a second, different, communication system for receiving data from the implant.

According to embodiments of the second part of aspect 252SE, the external device is configured for sending a first key to the implant using the first communication system, the first key being needed for decrypting the data.

According to embodiments of the second part of aspect 252SE, the external device is configured for sending a third key to the implant using the first communication system, the third key being generated by a second external device, separate from the external device or by another external device being a generator of the second key on behalf of the second external device, the third key being received at the external device and sent to the implant using the first communication system.

According to embodiments of the second part of aspect 252SE, the external device is further configured to: confirming the connection, via the first communication system, between the implant and the external device, wherein the external device is configured to communicate further data to the implant following positive confirmation.

According to embodiments of the second part of aspect 252SE, the further data comprises at least one of data for updating a control program running in the implant, and operation instructions for operating the implant.

According to embodiments of the second part of aspect 252SE, the first communication system is configured for wireless communication using a first network protocol, wherein the second communication system is configured for wireless communication using a second network protocol.

According to embodiments of the second part of aspect 252SE, the first or second network protocol is a proprietary network protocol, wherein the other network protocol is a standard network protocol.

According to embodiments of the second part of aspect 252SE, the data received at the external device is encrypted.

According to embodiments of the second part of aspect 252SE, the second communication system is configured to receive a first key from the implant, wherein the external device comprises a computing unit configured for: deriving a combined key using the first key with a second key held by the external device, and decrypting the encrypted data received from the implant using the combined key.

According to embodiments of the second part of aspect 252SE, the data received from the implant via the second communication system comprises feedback signals from the implant including one or more from the list of physiological or physical sensor parameters related to the status of the body of the patient, and physical or functional parameters related to status of the implant.

In a third part of aspect 252SE, there is provided an implant configured for communication with an external device, when the implant is implanted in a patient, the implant comprising:
a first communication system for receiving data from the external device,
a second, different, communication system for: sending data to the external device.

According to embodiments of the third part of aspect 252SE, the first communication system is further configured for receiving, by the first communication system, a first key from the implant, wherein the implant further comprises a computing unit configured to:
deriving a combined key using the first key with a second key held by the implant,
decrypting the data using the combined key,
using the decrypted data for instructing the implant.

According to embodiments of the third part of aspect 252SE, the implant is configured for receiving, from the external device or a second external device separate from the external device, a third key wherein the computing unit is configured to deriving the combined key using the first, second and third keys, and decrypting the data, in the implant, using the combined key.

According to embodiments of the third part of aspect 252SE, the implant further comprising a computing unit configured for:
confirming the connection via the first communication system between the implant and the external device, and as a result of the confirmation, instructing the implant based on the data sent from the external device.

According to embodiments of the third part of aspect 252SE, the computing unit is configured for altering the operation of the implant based on the data decrypted in the implant.

According to embodiments of the third part of aspect 252SE, the first communication system is configured for wireless communication using a first network protocol, wherein the second communication system is configured for wireless communication using a second network protocol.

According to embodiments of the third part of aspect 252SE, wherein the first or second network protocol is a proprietary network protocol, wherein the other network protocol is a standard network protocol.

According to embodiments of the third part of aspect 252SE, the data transmitted to the external device is encrypted, wherein the implant is further configured to transmit a first part of a key to the external device, the first part of the key being a part of a combined key to be used for decrypting the transmitted encrypted data.

According to embodiments of the third part of aspect 252SE, the data transmitted to the external device comprises feedback signals from the implant including one or more from the list of: physiological or physical sensor parameters related to the status of the body of the patient, and physical or functional parameters related to status of the implant.

According to embodiments of the first part of aspect 252SE, the data sent from the external device to the implant is encrypted data.

According to embodiments of the first part of aspect 252SE, the first communication system is a conductive communication system.

According to embodiments of the second part of aspect 252SE, the data sent to the implant is encrypted data.

According to embodiments of the second part of aspect 252SE, the first communication system is a conductive communication system.

According to embodiments of the third part of aspect 252SE, the data received from the external device is encrypted data.

According to embodiments of the third part of aspect 252SE, the first communication system is a conductive communication system.

According to embodiments the implant according to at least a part of; any one of method embodiments of the first part of aspect 252SE, and/or any of the implant embodiments of the third part of aspect 252SE, and/or any one of the computer product embodiments of the fourth part of aspect 252SE, wherein the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter, an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

In a fourth part of aspect 252SE, there is provided a computer program product of, or arranged to run on, an external device configured for communication with an implant when implanted in a patient, the external device comprising
a. a first communication system, wherein the computer program product is configured to cause the first communication system to be used for sending data to the implant,
b. a second, different, communication system wherein the computer program product is configured to cause the second communication system to be used for receiving data from the implant.

According to embodiments of the fourth part of aspect 252SE the computer program product is configured to cause the external device to send a first key to the implant using the first communication system, the first key being needed for decrypting the data.

According to embodiments of the fourth part of aspect 252SE the computer program product is configured to cause the external device to send a third key to the implant using the first communication system, the third key being generated by a second external device, separate from the external device or by another external device being a generator of the second key on behalf of the second external device, the third key being received at the external device and sent to the implant using the first communication system.

According to embodiments of the fourth part of aspect 252SE the computer program product is configured to confirm the connection, via the first communication system, between the implant and the external device, wherein the computer program product is further configured to cause the external device to communicate further data to the implant following positive confirmation.

According to embodiments of the fourth part of aspect 252SE the further data comprises at least one of:
a. data for updating a control program running in the implant, and
a. operation instructions for operating the implant.

According to embodiments of the fourth part of aspect 252SE the first communication system is configured for wireless communication using a first network protocol, wherein the second communication system is configured for wireless communication using a second network protocol.

According to embodiments of the fourth part of aspect 252SE the first or second network protocol is a proprietary network protocol, wherein the other network protocol is a standard network protocol.

According to embodiments of the fourth part of aspect 252SE the data received at the external device is encrypted.

According to embodiments of the fourth part of aspect 252SE the second communication system is configured to receive a first key from the implant, wherein the external device comprises a computing unit wherein the computer program product is configured to cause the computing unit to:
derive a combined key using the first key and a second key held by the external device, and
decrypt the encrypted data received from the implant using the combined key.

According to embodiments of the fourth part of aspect 252SE the data received from the implant via the second communication system comprises feedback signals from the implant including one or more from the list of: physiological or physical sensor parameters related to the status of the body of the patient, and physical or functional parameters related to status of the implant.

According to embodiments of the fourth part of aspect 252SE the data sent to the implant is encrypted data.

According to embodiments of the fourth part of aspect 252SE the first communication system is a conductive communication system.

According to embodiments the implant according to at least a part of; any one of method embodiments of the first part of aspect 252SE, and/or any of the implant embodiments of the third part of aspect 252SE, and/or any one of the computer product embodiments of the fourth part of aspect 252SE comprising an internal control unit adapted to be involved in at least a part of the actions performed by the implant in at least a part of any one of the other embodiments of aspect 252SE.

### Aspect 253SE Passive Proxy—Passive Proxy—Embodiments of Aspect 253SE of the Disclosure

In a first part of tenth aspect, there is provided an external device for relaying communication between a second external device and an implant implanted in a patient. The external device comprises a wireless transceiver configured for wireless communication with the second external device and the implant. The wireless transceiver is configured to receive an instruction from the second external device. The wireless transceiver is configured to transmit the instruction to the implant. The external device further comprises a verification unit. The verification unit is configured to:
upon authentication of the relaying functionality of the external device, cause the wireless transceiver to transmit the instruction to the implant; and
   upon non-authentication or failed authentication of the relaying functionality of the external device, cause the external device to hold the instructions.

According to some embodiments of the first part of aspect 253SE the user is the patient in which the implant is implanted.

According to some embodiments of the first part of aspect 253SE the authentication input is a parameter of the patient.

According to some embodiments of the first part of aspect 253SE the user is a caregiver.

According to some embodiments of the first part of aspect 253SE the authentication input is a parameter of the caregiver.

According to some embodiments of the first part of aspect 253SE the authentication input is a code.

According to some embodiments of the first part of aspect 253SE the wireless transceiver is configured to receive the instruction from the second external device communicated using a first network protocol.

According to some embodiments of the first part of aspect 253SE the wireless transceiver is configured to transmit the instruction to the implant communicated using a second network protocol.

According to some embodiments of the first part of aspect 253SE the first network protocol is a standard network protocol from the list of:
Radio-frequency type protocol
Radio-frequency identification (RFID) type protocol
Wireless local-area network (WLAN)
Bluetooth
Bluetooth low energy (BLE)
Near-field communication (NFC)
3G/4G/5G
GSM

According to some embodiments of the first part of aspect 253SE the second network protocol is a proprietary network protocol.

According to some embodiments of the first part of aspect 253SE the instruction received at the external device is encrypted. The external device may be configured to transmit the instruction to the implant without decrypting the instruction

According to some embodiments of the first part of aspect 253SE the second external device comprises an instruction provider adapted to receive instructions from a caregiver generating at least one component of the instruction.

According to some embodiments of the first part of aspect 253SE the external device is further adapted to receive authentication input from the caregiver, comprising at least one of a code and a parameter of the caregiver.

According to some embodiments of the first part of aspect 253SE a code is generated by the instruction provider.

According to some embodiments of the first part of aspect 253SE the authentication input comprises a single use code.

According to some embodiments of the first part of aspect 253SE the external device is configured to be placed in electrical connection with a conductive member, for conductive communication with the implant.

In a second part of tenth aspect, there is provided a method for relaying communication between a second external device and an implant implanted in a patient via a wireless transceiver of an external device.

The method comprises the steps of: Receiving, by the wireless transceiver, an instruction from the second external device communicated using a first network protocol.

Receiving, by a verification unit, authentication input from a user.

Authenticating a relaying functionality of the external device based on the authentication input.

Upon authentication of the relaying functionality of the external device, transmitting, by the wireless transceiver, the instruction to the implant using a second network protocol,

Upon non-authentication or failed authentication of the relaying functionality of the external device, holding the instructions at the external device. According to some embodiments of the second part of aspect 253SE the user is the patient in which the implant is implanted and wherein the implant is using a second network protocol to transmit that the relaying functionality of the external device is authenticated.

According to some embodiments of the second part of aspect 253SE the authentication input is a parameter of the patient.

According to some embodiments of the second part of aspect 253SE the user is a caregiver.

According to some embodiments of the second part of aspect 253SE the authentication input is a parameter of the caregiver.

According to some embodiments of the second part of aspect 253SE the authentication input is a code.

According to some embodiments of the second part of aspect 253SE the first network protocol is a standard network protocol from the list of:
Radio-frequency type protocol
RFID type protocol
WLAN
Bluetooth
BLE
NFC
3G/4G/5G
GSM

According to some embodiments of the second part of aspect 253SE the second network protocol is a proprietary network protocol.

According to some embodiments of the second part of aspect 253SE the instruction received at the external device is encrypted. The step of transmitting the instruction to the implant may then be performed without decrypting the instruction at the external device.

According to some embodiments of the second part of aspect 253SE the method further comprises the steps of: Receiving, by an instruction provider of the second external device, instructions from a caregiver.

Generating at least one component of the instruction.

According to some embodiments of the second part of aspect 253SE the method further comprises providing, by the caregiver, authentication input comprising at least one of a code and a parameter of the caregiver.

According to some embodiments of the second part of aspect 253SE the method further comprises generation of a code by the instruction provider.

According to some embodiments of the second part of aspect 253SE the authentication input comprises a single use code.

According to some embodiments of the second part of aspect 253SE the method further comprises placing a conductive member, configured to be in connection with the external device, in electrical connection with a skin of the patient for conductive communication with the implant.

In a third part of tenth aspect, there is provided a system comprising an external device according to the first part of aspect 253SE and an implant implanted in a patient. The system further comprises a conductive member configured to be in electrical connection with the external device. The conductive member is configured to be placed in electrical connection with a skin of the patient for conductive communication with the implant.

According to some embodiments of the first part of aspect 253SE the external device is configured to decrypt the communication from the second external device at the external device and wherein the external device is further configured to transmit the decrypted communication to the implant via a short range communication method.

In a fourth part of aspect 253SE, there is provided a computer program product of, or arranged to run on, an external device for relaying communication between a second external device and an implant implanted in a patient, the external device comprising:
a wireless transceiver configured for wireless communication with the second external device and the implant, wherein the computer program product is configured to cause the wireless transceiver to receive an instruction from the second external device, wherein the computer program product is configured to cause the wireless transceiver to transmit the instruction to the implant, and
a verification unit configured to receive authentication input from a user, for authenticating a relaying functionality of the external device, wherein the computer program product is configured to:
   upon authentication of the relaying functionality of the external device, cause the wireless transceiver to transmit the instruction to the implant; and
   upon non-authentication or failed authentication of the relaying functionality of the external device, cause the external device to hold the instructions.

According to some embodiments of the fourth part of aspect 253SE the user is the patient in which the implant is implanted.

According to some embodiments of the fourth part of aspect 253SE the authentication input is a parameter of the patient.

According to some embodiments of the fourth part of aspect 253SE the authentication input is a code.

According to some embodiments of the fourth part of aspect 253SE the user is a caregiver.

According to some embodiments of the fourth part of aspect 253SE the authentication input is a parameter of the caregiver.

According to some embodiments of the fourth part of aspect 253SE the authentication input is a code.

According to some embodiments of the fourth part of aspect 253SE the wireless transceiver is configured to receive the instruction from the second external device communicated using a first network protocol.

According to some embodiments of the fourth part of aspect 253SE the wireless transceiver is configured to transmit the instruction to the implant communicated using a second network protocol.

According to some embodiments of the fourth part of aspect 253SE the first network protocol is a standard network protocol from the list of: Radio-frequency type protocol,
RFID type protocol,
WLAN,
Bluetooth,
BLE,
NFC,
3G/4G/5G, and
GSM.

According to some embodiments of the fourth part of aspect 253SE the second network protocol is a proprietary network protocol.

According to some embodiments of the fourth part of aspect 253SE the instruction received at the external device is encrypted, and wherein the computer program product is configured to cause the external device to transmit the instruction to the implant without decrypting the instruction.

According to some embodiments of the fourth part of aspect 253SE the second external device comprises an instruction provider wherein the computer program product is configured to cause the instruction provider to receive instructions from a caregiver generating at least one component of the instruction.

According to some embodiments of the fourth part of aspect 253SE the computer program product is further configured to cause the external device to receive authentication input from the caregiver, comprising at least one of a code and a parameter of the caregiver.

According to some embodiments of the fourth part of aspect 253SE the computer program product is configured to cause a code to be generated by the instruction provider.

According to some embodiments of the fourth part of aspect 253SE the authentication input comprises a single use code.

According to some embodiments of the fourth part of aspect 253SE the computer program product is configured to cause the external device to decrypt the communication from the second external device at the external device and wherein the computer program product is further configured to cause the external device to transmit the decrypted communication to the implant via a short range communication method.

According to some embodiments the implant according to at least a part of any one of embodiments of aspect 253SE, comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments the implant according to, or presented in, any one of the embodiments of aspect 253SE, comprises an internal control unit adapted to be involved in at least a part of the actions performed by the implant in at least a part of any one of the embodiments of aspect 251SE.

### Aspect 254SE Automatic Update—Automatic Update of Control Program of Implant—Embodiments of Aspect 254SE of the Disclosure

In a first part of aspect 254SE, there is provided a method for updating a control program adapted to run in a computing unit of an implant when implanted in a patient, the method comprising: receiving data by the computing unit, and updating, by the computing unit, the control program on the basis of the received data.

According to embodiments of the first part of aspect 254SE, the method further comprises: transmitting data from the implant to an external device, updating the control program, at the external device, on the basis of the received data, wherein the data received by the computing unit comprises the updated control program.

According to embodiments of the first part of aspect 254SE, the data transmitted from the implant comprises at least one physiological parameter of the patient.

According to embodiments of the first part of aspect 254SE, the data transmitted from the implant comprises at least one functional parameter of the implant.

According to embodiments of the first part of aspect 254SE, the method further comprises: sensing at least one parameter using an implantable sensor, wherein the received data by the computing unit comprises said at least one sensed parameter, and updating, by the computing unit, the control program on the basis of the at least one sensed parameter.

According to embodiments of the first part of aspect 254SE, the at least one parameter comprises at least one physiological parameter of the patient.

According to embodiments of the first part of aspect 254SE, the at least one parameter comprises at least one functional parameter of the implant.

According to embodiments of the first part of aspect 254SE, the method further comprises: the patient or a caregiver of the patient controlling the computing unit using at least one of an implantable manual receiver, an implantable switch and a remote control, the patient or caregiver providing feedback related to the operation of the implant, wherein the data received by the computing unit comprises said feedback, and the computing unit updating the control program on the basis of the patient feedback.

According to embodiments of the first part of aspect 254SE, the method further comprises: receiving feedback from at least one of, the patient in whom the implant is implanted and at least one sensor, in response to the control program controlling the implant, and updating, by the computing unit, the control program on the basis of the received feedback.

According to embodiments of the first part of aspect 254SE, the data received by the computing unit comprises said feedback.

According to embodiments of the first part of aspect 254SE, the method further comprises the steps of—updating the control program, at an external device, on the basis of the said feedback, wherein the data received by the computing unit comprises the updated control program.

According to embodiments of the first part of aspect 254SE, the step of updating the control program comprises adjusting at least one parameter of the implant.

According to embodiments of the first part of aspect 254SE, the method further comprises the steps of transmitting the received feedback to an external device, and wherein the received data by the computing unit comprises calibration parameters transmitted from the external device, said calibration parameters based on the feedback provided to the external device.

According to embodiments of the first part of aspect 254SE, the method further comprises the steps of—receiving authentication input from a user for authenticating the updating of the control program, as a result of the authentication input, updating the control program by the computing unit.

According to embodiments of the first part of aspect 254SE, the implant is wirelessly connected to an external device, the external device configured to relay communication between a second external device and an implant, the method comprising the steps of receiving, by a wireless transceiver in the external device, an instruction from the second external device communicated using a first network protocol, receiving, by a verification unit of the external device, authentication input from a user, authenticating the relay functionality of the external device based on the authentication input, and transmitting, by the wireless transceiver, the instruction to the implant, only if the relaying functionality of the external device is authenticated, using a second network protocol, wherein the data received by the computing unit comprises the instructions.

According to embodiments of the first part of aspect 254SE, the instructions comprises one of the updated control program, and calibration parameters of the implant.

According to embodiments of the first part of aspect 254SE, the authentication input is a parameter of the patient.

According to embodiments of the first part of aspect 254SE, the authentication input is a code.

According to embodiments of the first part of aspect 254SE, the first network protocol is a standard network protocol from the list of a Radio Frequency type protocol, a RFID type protocol, a WLAN type protocol, a Bluetooth type protocol, a BLE type protocol, a NFC type protocol, a 3G/4G/5G type protocol, a GSM type protocol.

According to embodiments of the first part of aspect 254SE, the second network protocol is a proprietary network protocol.

According to embodiments of the first part of aspect 254SE, the data received by the computing unit is encrypted, the method further comprising the steps of receiving, by the computing unit, at least one key, and decrypting the encrypted data using the at least one key.

In a second part of aspect 254SE, there is provided an implant, configured to update a control program adapted to run in a computing unit of the implant when implanted in a patient, the computing unit being configured for: receiving data, and updating the control program on basis of the received data. According to embodiments of the second part of aspect 254SE, the implant is further configured for: transmitting data, using a transceiver, from the implant to an external device, as a response to the transmitted data, receiving, by the transceiver, an updated control program from the external device.

According to embodiments of the second part of aspect 254SE, the implant further comprises a sensor for sensing at least one physiological parameter of the patient, wherein the data transmitted from the implant comprises at least one physiological parameter of the implant.

According to embodiments of the second part of aspect 254SE, the data transmitted from the implant comprises at least one functional parameter of the implant.

According to embodiments of the second part of aspect 254SE, the implant is further in communication with an implantable sensor adapted to sense at least one parameter, wherein the received data by the computing unit comprises said at least one sensed parameter, wherein the computing unit is configured for updating, the control program on the basis of the at least one sensed parameter.

According to embodiments of the second part of aspect 254SE, the at least one parameter comprises at least one physiological parameter of the implant.

According to embodiments of the second part of aspect 254SE, the at least one parameter comprises at least one functional parameter of the implant.

According to embodiments of the second part of aspect 254SE, the computing unit is configured to be controlled by at least one of an implantable manual receiver, an implantable switch or a remote control to received feedback from the patient, wherein the computing unit is configured to update the control program on the basis of the patient feedback.

According to embodiments of the second part of aspect 254SE, the implant is configured to receive feedback from at least one of, the patient in whom the implant is implanted and at least one sensor, in response to the control program controlling the implant, wherein the computing unit is configured to update the control program on the basis of the received feedback.

According to embodiments of the second part of aspect 254SE, the implant is configured to transmit the received feedback to an external device, and as a response there to, receiving data by the computing unit comprising calibration parameters transmitted from the external device, said calibration parameters based on the feedback provided to the external device.

According to embodiments of the second part of aspect 254SE, the computing unit is configured to update the control program by adjusting at least one parameter of the implant.

According to embodiments of the second part of aspect 254SE, the computing unit is configured to receive authentication input from a user for authenticating the updating of the control program, and as a result of the authentication input, update the control program by the computing unit.

According to embodiments of the second part of aspect 254SE, wherein the data received by the computing unit is encrypted, wherein the computing unit is further configured for: receiving at least one key, decrypting the encrypted data using the at least one key.

In a third part of aspect 254SE, there is provided a system comprising an implant according to the second part of aspect 254SE, wirelessly connected to an external device, the external device configured to relay communication between a second external device and an implant, the external device comprising:
a wireless transceiver configured for wireless communication with the second external device and the implant, the wireless transceiver configured to receive an instruction from the second external device communicated using a first network protocol, wherein the wireless transceiver is configured to transmit the instruction to the implant using a second network protocol, and
a verification unit configured to receive authentication input from a user, for authenticating the relaying functionality of the external device, wherein the wireless transceiver is configured to transmit the instruction to the implant only if the relaying functionality of the external device is authenticated, wherein the data received by the computing unit of the implant comprises the instructions.

According to embodiments of the third part of aspect 254SE, the instructions comprises one of the updated control program, and calibration parameters of the implant.

According to embodiments of the third part of aspect 254SE, the authentication input is a parameter of the patient.

According to embodiments of the third part of aspect 254SE, the authentication input is a code.

According to embodiments of the third part of aspect 254SE, the first network protocol is a standard network protocol from the list of: a Radio Frequency type protocol, a RFID type protocol, a WLAN type protocol, a Bluetooth type protocol, a BLE type protocol, a NFC type protocol, a 3G/4G/5G type protocol, a GSM type protocol.

According to embodiments of the third part of aspect 254SE, the second network protocol is a proprietary network protocol.

According to embodiments of the first part of aspect 254SE a first communication system is used for receiving data by the computing unit of the implant, and wherein a second communication system is used for transmitting data from the implant to the external device.

According to embodiments of the first part of aspect 254SE the method further comprises relaying data to the second external device and receiving the updated control program at the second external device.

According to embodiments of the first part of aspect 254SE a caregiver transmits data to the implant from a second external device directly or via the external device.

According to embodiments of the first part of aspect 254SE a connection between the implant and the external device is authenticated by a conductive communication or connection between the implant and the external device.

According to embodiments of the second part of aspect 254SE the implant comprises at least one of:

According to some embodiments the implant according to at least a part of any one of embodiments of aspect 254SE, such as the second part of aspect 254SE, comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments the implant according to, or presented in, any one of the embodiments of aspect 254SE, e.g. the second part of aspect 254SE, comprises an internal control unit adapted to be involved in at least a part of the actions performed by the implant in at least a part of any one of the embodiments of aspect 254SE.

### Aspect 255SE Information from Implant—Information from Implant—Embodiments of Aspect 255SE of the Disclosure

In a first part of aspect 255SE there is provided an implant. The implant comprises at least one sensor for sensing at least one physiological parameter of the patient and a communication unit configured to transmit the sensed parameter from the body of the patient to an external device. The implant comprises at least one vascular portion configured to be placed in proximity to a blood vessel of the patient, and the vascular portion comprises the sensor. The sensor is a sensor configured to sense at least one parameter related to the blood of the patient.

According to some embodiments of the first part of aspect 255SE the vascular portion comprises at least one needle for extracting blood from the blood vessel for transport to the at least one sensor.

According to some embodiments of the first part of aspect 255SE the vascular portion further comprises a needle operating device configured to displace the needle such that the needle can change from extracting blood at a first site to extracting blood at a second site.

According to some embodiments of the first part of aspect 255SE the sensor is an optical sensor configured to optically sense at least one parameter of the blood of the patient.

According to some embodiments of the first part of aspect 255SE the sensor is configured for spectrophotometry.

According to some embodiments of the first part of aspect 255SE the optical sensor is configured to sense visible light.

According to some embodiments of the first part of aspect 255SE the optical sensor is configured to sense UV light.

According to some embodiments of the first part of aspect 255SE the optical sensor is configured to sense IR radiation.

According to some embodiments of the first part of aspect 255SE the at least one sensor is configured to sense at least one of: oxygen saturation, blood pressure, a parameter related to the function of the liver, a parameter related to the existence of cancer, a parameter related to the bile function, glucose, lactate, pyruvate, prostate-specific antigen, cholesterol level, potassium, sodium, cortisol, adrenalin, ethanol, parameters relating to blood composition, platelets, white blood cells, red blood cells, viscosity, a parameter relating to flux, a parameter relating to the direction of flow, a parameter relating to flow velocity, blood plasma concentration, a parameter relating to hormones, a parameter relating to enzyme activity, calcium, iron, iron-binding capacity, transferrin, ferritin, ammonia, copper, ceruloplasmin, phosphate, zinc, magnesium, pH, oxygen partial pressure, carbon dioxide, bicarbonate, protein(s), a parameter relating to blood lipids, tumor markers, vitamins, toxins, antibodies, and electrolytes, a drug level, the level of a drug transposed into different a substance, a treatment marker level, an antigen level, an antibody level, an immunoglobin level.

According to some embodiments of the first part of aspect 255SE the at least one sensor is configured to sense at least one of: a parameter related to the effect of a therapeutic treatment and the presence of a pharmaceutical or a substance caused by the pharmaceutical.

According to some embodiments of the first part of aspect 255SE the at least one sensor is configured to sense the presence of at least one of: an antibiotic pharmaceutical, a chemotherapy pharmaceutical and insulin or a substance caused by anyone of the preceding.

According to some embodiments of the first part of aspect 255SE the at least one sensor is configured to sense a parameter related the effect of at least one of: a cancer treatment and an antibiotic treatment.

In a second part of aspect 255SE there is provided an implant, wherein the implant comprises at least one sensor for sensing at least one physiological parameter of the patient and a communication unit configured to transmit the sensed parameter from the body of the patient to an external device. The implant comprises at least one food passageway portion configured to be placed in proximity to the food passageway of the patient, and wherein the food passageway portion comprises the sensor, and wherein the sensor is a sensor configured to sense at least one parameter related to the food passageway of the patient.

According to some embodiments of the second part of aspect 255SE the sensor is a sensor configured to sense at least one of intestinal activity, activity of the stomach and activity of the esophagus.

According to some embodiments of the second part of aspect 255SE the sensor is at least one of an accelerometer, a motility sensor, and a strain sensor.

According to some embodiments of the second part of aspect 255SE the sensor is a sensor configured to sense an electrical parameter.

According to some embodiments of the second part of aspect 255SE the sensor is a sensor configured to sense any parameter relating to the contents of at least one of: an intestine, the stomach, and the esophagus.

According to some embodiments of the second part of aspect 255SE the food passageway portion comprises at least one needle for extracting contents from the food passageway for transport to the at least one sensor.

According to some embodiments of the second part of aspect 255SE the food passageway portion further comprises a needle operating device configured to displace the needle such that the needle can change from extracting contents from the food passageway at a first site to extracting contents of the food passageway at a second site.

According to some embodiments of the second part of aspect 255SE the sensor is an optical sensor configured to optically sense at least one parameter of the food passageway of the patient.

According to some embodiments of the second part of aspect 255SE the optical sensor is configured for spectrophotometry.

According to some embodiments of the second part of aspect 255SE the optical sensor is configured to sense visible light.

According to some embodiments of the second part of aspect 255SE the optical sensor is configured to sense UV light.

According to some embodiments of the second part of aspect 255SE the optical sensor is configured to sense IR radiation.

According to some embodiments of the second part of aspect 255SE the sensor is a sensor configured to directly or indirectly and precisely or approximately sense the passage of food down the food passageway, including at least one of solid food passing down the food passageway, liquid passing down the food passageway, and the number of swallowing of contents passing down the food passageway of at least one of: an intestine, the stomach and the esophagus.

According to some embodiments of the second part of aspect 255SE the sensor is an audio sensor configured to sense a sound parameter of the food passageway of the patient.

According to some embodiments of the second part of aspect 255SE the sensor is an audio sensor configured to sense a sound parameter of the intestine of the patient.

In a third part of aspect 255SE there is provided an implant, wherein the implant comprises at least one sensor for sensing at least one physiological parameter of the patient and a communication unit configured to transmit the sensed parameter from the body of the patient to an external device. The at least one sensor is an ultrasound sensor configured to sense the at least one parameter of the patient using ultrasound.

According to some embodiments of the third part of aspect 255SE the implant comprises a cardiac portion, and the cardiac portion comprises the ultrasound sensor, and the ultrasound sensor is configured to sense at least one parameter related to the heart of the patient.

According to some embodiments of the third part of aspect 255SE the ultrasound sensor is configured to sense the blood flow in the heart.

According to some embodiments of the third part of aspect 255SE the ultrasound sensor is configured to sense the presence of fluid in the pericardial cavity.

According to some embodiments of the third part of aspect 255SE the ultrasound sensor is configured to sense the presence of an assembly of fluid in the body of the patient.

According to some embodiments of the third part of aspect 255SE the ultrasound sensor is configured to sense the level of urine in the urinary bladder.

In a fourth part of aspect 255SE there is provided an implant, wherein the implant comprises at least one sensor for sensing at least one physiological parameter of the patient and a communication unit configured to transmit the sensed parameter from the body of the patient to an external device, wherein the implant comprises a cardiac portion. The cardiac portion comprises the sensor. The sensor is configured to sense at least one parameter related to the heart of the patient.

According to some embodiments of the fourth part of aspect 255SE the sensor is configured to sense at least one parameter related to the electrical activity of the heart.

According to some embodiments of the fourth part of aspect 255SE the sensor is configured to sense at least one sound parameter related to the heart.

In a fifth part of aspect 255SE there is provided an implant, wherein the implant comprises at least one sensor for sensing at least one physiological parameter of the patient and a communication unit configured to transmit the sensed parameter from the body of the patient to an external device, wherein the implant comprises a pulmonary portion. The pulmonary portion comprises the sensor, and the sensor is configured to sense at least one parameter related to the lungs of the patient.

According to some embodiments of the fifth part of aspect 255SE the sensor is a sensor configured to sense respiratory activity.

According to some embodiments of the fifth part of aspect 255SE the sensor is at least one of an accelerometer, a motility sensor, and a strain sensor.

According to some embodiments of the fifth part of aspect 255SE the sensor is an optical sensor configured to optically sense at least one parameter of the lungs of the patient.

According to some embodiments of the fifth part of aspect 255SE the sensor is an audio sensor configured to sense a sound parameter of the lungs of the patient.

In a sixth part of aspect 255SE there is provided an implant, wherein the implant comprises at least one sensor for sensing at least one physiological parameter of the patient and a communication unit configured to transmit the sensed parameter from the body of the patient to an external device, wherein the implant comprises a urinary portion. The urinary portion comprises the sensor, and the sensor is configured to sense at least one parameter related to the urine bladder of the patient.

According to some embodiments of the sixth part of aspect 255SE the sensor is an optical sensor configured to optically sense at least one parameter of the urine bladder of the patient.

According to some embodiments of the sixth part of aspect 255SE the sensor is a sensor configured to sense activity of the urinary bladder.

According to some embodiments of the sixth part of aspect 255SE the sensor is at least one of an accelerometer, a motility sensor, and a strain sensor.

In a seventh part of aspect 255SE there is provided an implant, wherein the implant comprises at least one sensor for sensing at least one physiological parameter of the patient and a communication unit configured to transmit the sensed parameter from the body of the patient to an external device, wherein the at least one sensor is an audio sensor configured to sense the at least one audio parameter of the patient.

According to some embodiments of the seventh part of aspect 255SE the sensor is a sensor configured to sense an audio parameter related to an activity of the gastrointestinal system.

According to some embodiments of the seventh part of aspect 255SE the sensor is a sensor configured to sense an audio parameter related to an activity of the lungs of the patient.

According to some embodiments of the seventh part of aspect 255SE the sensor is a sensor configured to sense an audio parameter related to an activity of the heart of the patient.

According to some embodiments of the seventh part of aspect 255SE the sensor is a sensor configured to sense an audio parameter related to the voice of the patient.

In an eighth part of aspect 255SE there is provided an implant, wherein the implant comprises at least one sensor for sensing at least one physiological parameter of the patient and a communication unit configured to transmit the sensed parameter from the body of the patient to an external device, wherein the at least one sensor is an audio sensor configured to sense the at least one audio parameter of the patient.

According to some embodiments of the eighth part of aspect 255SE the sensor is a sensor configured to sense an audio parameter related to an activity of the gastrointestinal system.

According to some embodiments of the eighth part of aspect 255SE the sensor is a sensor configured to sense an audio parameter related to an activity of the lungs of the patient.

According to some embodiments of the eighth part of aspect 255SE the sensor is a sensor configured to sense an audio parameter related to an activity of the heart of the patient.

According to some embodiments of the eighth part of aspect 255SE the sensor is a sensor configured to sense an audio parameter related to the voice of the patient.

In a ninth part of aspect 255SE there is provided a system comprising an implant, implanted in a patient, an external device, and a second external device. The external device is configured to transmit data pertaining to the sensed parameter to the second external device. The external device is configured to add information to the data pertaining to the sensed parameter before transmitting to the second external device.

According to some embodiments of the ninth part of aspect 255SE the external device comprises a sensor for recording the information to be added to the data pertaining to the sensed parameter.

According to some embodiments of the ninth part of aspect 255SE the sensor comprises a thermometer or a geographical positioning sensor such as a global navigation satellite system, GNSS, receiver.

According to some embodiments of the ninth part of aspect 255SE the external device is configured to automatically add the information to the data pertaining to the sensed parameter.

According to some embodiments of the ninth part of aspect 255SE the external device is configured to, upon a manual input from a user, add the information to the data pertaining to the sensed parameter.

According to some embodiments of the ninth part of aspect 255SE the information added comprises at least one of:
a weight of the patient,
a height of the patient,
a body temperature of the patient,
eating habits of the patient,
physical exercise habits of the patient,
toilet habits of the patient,
an outside or external temperature of the patient, and
geographic position data of the patient.

According to some embodiments of the first through eighth parts of aspect 255SE the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to some embodiments of the first through eighth parts of aspect 255SE the implant comprises an internal control unit adapted to be involved in at least a part of the actions performed by the implant in at least a part of any one of the embodiments of aspect 255SE.

### Aspect 256SE Device Synchronization Patient Parameter—Authenticating a Connection Between an Implant and the External Device Using a Patient Parameter—Embodiments of Aspect 256SE of the Disclosure

In a first part of aspect 256SE, there is provided a method of authenticating a connection between an implant implanted in a patient, and an external device. The method comprises establishing a connection between the external device and the implant, measuring a parameter of the patient, by the implant, measuring the parameter of the patient, by the external device, comparing the parameter measured by the implant to the parameter measured by the external device, and performing authentication of the connection based on the comparison.

According to some embodiments of the first part of aspect 256SE the method further comprises the step of transmitting the parameter measured by the external device from the external device to the implant, wherein the comparison is performed by the implant.

According to some embodiments of the first part of aspect 256SE the method further comprises the step of transmitting the parameter measured by the implant from the implant to the external device, wherein the comparison is performed by the external device.

According to some embodiments of the first part of aspect 256SE the parameter of the patient is related to a pulse of the patient.

According to some embodiments of the first part of aspect 256SE the parameter of the patient is related to a blood oxygen saturation of a patient.

According to some embodiments of the first part of aspect 256SE the parameter of the patient is related to a respiration rate of the patient.

According to some embodiments of the first part of aspect 256SE the parameter of the patient is related to a temperature of the patient.

According to some embodiments of the first part of aspect 256SE the parameter of the patient is related to at least one sound of the patient.

According to some embodiments of the first part of aspect 256SE the parameter of the patient is related to at least one physical movement of the patient.

According to some embodiments of the first part of aspect 256SE the measured parameter at the implant is provided with a timestamp and the measured parameter at the external device is provided with a timestamp. The step of comparing the parameter measured at the implant to the parameter measured by the external device comprises comparing the timestamp provided by the implant to the timestamp provided by the external device.

According to some embodiments of the first part of aspect 256SE the method further comprises the step of synchronizing a clock of the implant with a clock of the external device.

According to some embodiments of the first part of aspect 256SE the step of comparing the parameter measured by the implant to the parameter measured by the external device comprises calculating a difference value between the parameter measured by the implant and the parameter measured by the external device. The step of performing authentication comprises authenticating the connection if the difference value is less than a predetermined threshold difference value, and not authenticating the connection if the difference value equals or exceeds the predetermined threshold difference value.

According to some embodiments of the first part of aspect 256SE the method further comprises placing a conductive member, configured to be in connection with the external device, in electrical connection with a skin of the patient for conductive communication with the implant.

According to some embodiments of the first part of aspect 256SE the communication between the implant and the external device is a wireless communication.

According to some embodiments of the first part of aspect 256SE the communication between the implant and the external device is a conductive communication.

According to some embodiments of the first part of aspect 256SE further comprising the step of communicating further data between the implant and the external device following positive authentication.

According to some embodiments of the first part of aspect 256SE the method further comprising determining a cryptographic hash based on the parameter as measured by at least one of the external device and the implant, wherein the further data comprises the cryptographic hash.

According to some embodiments of the first part of aspect 256SE the further data is communicated from the external device to the implant, wherein the further data comprises at least one of data for updating a control program running in the implant, and operation instructions for operating the implant.

According to some embodiments of the first part of aspect 256SE wherein the further data is communicated from the implant to the external device, wherein the further data comprises data sensed by a sensor connected to the implant.

According to some embodiments of the first part of aspect 256SE wherein the comparison is performed by the implant. The method further comprises the step of continuously requesting by the external device, or receiving at the external device, information of an authentication status of the connection between the implant and the external device, and upon determining, at the external device, that the connection is authenticated, transmitting further data from the external device to the implant.

According to some embodiments of the first part of aspect 256SE the comparison is performed by the external device. The method further comprises the step of continuously requesting by the implant, or receiving at the implant, information of an authentication status of the connection between the implant and the external device, and upon determining, at the implant, that the connection is authenticated, transmitting further data from the implant to the external device.

In a second part of aspect 256SE, there is provided an implant, implanted in a patient, adapted for connection with an external device. The implant comprises a first sensor for measuring a parameter of the patient. The implant further comprises an internal computing unit. The internal computing unit is configured for receiving a parameter of the patient, from the external device. The internal computing unit is further configured for comparing the parameter measured by the implant to the parameter measured by the external device. The internal computing unit is further configured for performing authentication of the connection based on the comparison.

According to some embodiments of the second part of aspect 256SE the first sensor is configured to measure a pulse of the patient.

According to some embodiments of the second part of aspect 256SE the first sensor is configured to measure a respiration rate of the patient.

According to some embodiments of the second part of aspect 256SE the first sensor is configured to measure a temperature of the patient.

According to some embodiments of the second part of aspect 256SE the first sensor is configured to measure at least one sound of the patient.

According to some embodiments of the second part of aspect 256SE the first sensor is configured to measure at least one physical movement of the patient.

According to some embodiments of the second part of aspect 256SE the measured parameter, by the implant is provided with a timestamp. The measured parameter received from the external device is provided with a timestamp. The comparison of the parameter measured at the implant to the parameter measured by the external device comprises comparing the timestamp of the measured parameter by the implant to the timestamp of the measured parameter received from the external device.

According to some embodiments of the second part of aspect 256SE the implant comprises a clock, configured for synchronization with a clock of the external device.

According to some embodiments of the second part of aspect 256SE the internal computing unit is configured to calculate a difference value between the parameter measured by the implant and the parameter measured by the external device. The internal computing unit is further configured to authenticate the connection if the difference value is less than a predetermined threshold difference value, and to not authenticate the connection if the difference value equals or exceeds the predetermined threshold difference value

According to some embodiments of the second part of aspect 256SE the communication between the implant and the external device is a wireless communication.

According to some embodiments of the second part of aspect 256SE the communication between the implant and the external device is a conductive communication.

According to some embodiments of the second part of aspect 256SE the implant is configured to communicate further data to the external device following positive authentication.

According to some embodiments of the second part of aspect 256SE the implant is further configured to determine a cryptographic hash based on the parameter as measured by at least one of the external device and the implant, wherein the further data comprises the cryptographic hash.

According to some embodiments of the second part of aspect 256SE the further data comprises data sensed by the sensor or another sensor connected to the implant.

In a third part of aspect 256SE, there is provided an external device, adapted for connection with an implant, implanted in a patient. The external device comprises a second sensor for measuring a parameter of the patient, by the external device. The external device further comprises an external computing unit. The external computing unit is configured for receiving a parameter of the patient, from the implant. The external computing unit is further configured for comparing the parameter measured by the external device to the parameter measured by the implant. The external computing unit is further configured for performing authentication of the connection based on the comparison.

According to some embodiments of the third part of aspect 256SE the second sensor is configured to measure a pulse of the patient.

According to some embodiments of the third part of aspect 256SE the second sensor is configured to measure a respiration rate of the patient.

According to some embodiments of the third part of aspect 256SE the second sensor is configured to measure a temperature of the patient.

According to some embodiments of the third part of aspect 256SE the second sensor is configured to measure at least one sound of the patient.

According to some embodiments of the third part of aspect 256SE the second sensor is configured to measure at least one physical movement by the patient.

According to some embodiments of the third part of aspect 256SE the measured parameter, by the external device is provided with a timestamp. The measured parameter received from the implant is provided with a timestamp. The comparison of the parameter measured at the implant to the parameter measured by the external device comprises comparing the timestamp of the measured parameter received from the implant to the timestamp of the measured parameter by the external device.

According to some embodiments of the third part of aspect 256SE the external device comprises a clock, configured for synchronization with a clock of the implant.

According to some embodiments of the third part of aspect 256SE the external computing unit is configured to calculate a difference value between the parameter measured by the implant and the parameter measured by the external device. The external computing unit is further configured to authenticate the connection if the difference value is less than a predetermined threshold difference value, and to not authenticate the connection if the difference value equals or exceeds the predetermined threshold difference value.

According to some embodiments of the third part of aspect 256SE the external device is configured to communicate further data to the implant following positive authentication.

According to some embodiments of the third part of aspect 256SE the implant is further configured to determine a cryptographic hash based on the parameter as measured by at least one of the external device and the implant, wherein the further data comprises the cryptographic hash.

According to some embodiments of the third part of aspect 256SE the further data comprises at least one of: data for updating a control program running in the implant, and operation instructions for operating the implant.

According to some embodiments of the third part of aspect 256SE the external device further comprises a conductive member configured to be in electrical connection with the external device. The conductive member is configured to be placed in electrical connection with a skin of the patient for conductive communication with the implant.

In a fourth part of aspect 256SE, there is provided a computer program product comprising a computer-readable storage medium with instructions adapted to carry out at least parts of the method of the first part of aspect 256SE, when executed by a device having processing capability.

According to embodiments of the second part of aspect 256SE the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

According to embodiments of the second part of aspect 256SE the implant comprises an internal control unit adapted to be involved in at least a part of the actions performed by the implant in at least a part of any one of the embodiments of aspect 256SE.

In a fifth part of aspect 256SE, there is provided a computer program product of, or adapted to run on, an external device, adapted for connection with an implant, implanted in a patient, the external device comprising:
c. a second sensor wherein the computer program product is configured to cause the second sensor to measure a parameter of the patient by the external device, and
d. an external computing unit, wherein the computer program product is configured to cause the external computing unit to:
   i. receive a parameter of the patient, from the implant,
   ii. compare the parameter measured by the external device to the parameter measured by the implant, and
   iii. perform authentication of the connection based on the comparison.

According to embodiments of the fifth part of aspect 256SE the parameter of the patient comprises a pulse of the patient.

According to embodiments of the fifth part of aspect 256SE the parameter of the patient comprises a respiration rate of the patient.

According to embodiments of the fifth part of aspect 256SE the parameter of the patient comprises a temperature of the patient.

According to embodiments of the fifth part of aspect 256SE the parameter of the patient comprises at least one sound of the patient.

According to embodiments of the fifth part of aspect 256SE the parameter of the patient comprises at least one physical movement by the patient.

According to embodiments of the fifth part of aspect 256SE the measured parameter, by the external device is provided with a timestamp and the measured parameter received from the implant is provided with a timestamp, wherein the comparison of the parameter measured at the implant to the parameter measured by the external device comprises comparing the timestamp of the measured parameter received from the implant to the timestamp of the measured parameter by the external device.

According to embodiments of the fifth part of aspect 256SE the computer program product is configured to cause a clock of the external device, to be synchronized with a clock of the implant.

According to embodiments of the fifth part of aspect 256SE the computer program product is configured to cause the external computing unit to calculate a difference value between the parameter measured by the implant and the parameter measured by the external device, and wherein the computer program product is further configured to cause the external computing unit to authenticate the connection if the difference value is less than a predetermined threshold difference value, and to not authenticate the connection if the difference value equals or exceeds the predetermined threshold difference value.

According to embodiments of the fifth part of aspect 256SE the computer program product is configured to cause the external device to communicate further data to the implant following positive authentication.

According to embodiments of the fifth part of aspect 256SE the computer program product is further configured to determine a cryptographic hash based on the parameter as measured by at least one of the external device and the implant, wherein the further data comprises the cryptographic hash.

According to embodiments of the fifth part of aspect 256SE the further data comprises at least one of:
a. data for updating a control program running in the implant, and
b. operation instructions for operating the implant.

In a sixth part of aspect 256SE, there is provided an implant for authenticating a connection between an implant implanted in a patient, and an external device, the implant comprising:
a. a sensor adapted to detect a sensation related to the body, as authentication data
b. a storing unit adapted to store the authentication data related to the sensation,
c. a receiver adapted to receive input from the external device related to the sensation, resulting in input authentication data, and
d. authenticating the connection based on an analysis of the input authentication data and the authentication data.

According to embodiments of the sixth part of aspect 256SE the authentication data and/or input authentication data is configured to pertain to a pulse of the patient.

According to embodiments of the sixth part of aspect 256SE the authentication data and/or input authentication data is configured to pertain toa respiration rate of the patient.

According to embodiments of the sixth part of aspect 256SE the authentication data and/or input authentication data is configured to pertain to a temperature of the patient.

According to embodiments of the sixth part of aspect 256SE the authentication data is configured to pertain to at least one sound of the patient.

According to embodiments of the sixth part of aspect 256SE the authentication data and/or input authentication data is configured to pertain to at least one physical movement of the patient.

According to embodiments of the sixth part of aspect 256SE the authentication data and/or input authentication data are provided with a timestamp, wherein the comparison of the authentication data measured at the implant to the input authentication data measured by the external device comprises comparing the timestamp of the measured parameter by the implant to the timestamp of the measured parameter received from the external device.

According to embodiments of the sixth part of aspect 256SE the implant comprises a clock, configured for synchronization with a clock of the external device.

According to embodiments of the sixth part of aspect 256SE the implant comprises an internal computing unit configured to calculate a difference value between the authentication data measured by the implant and the input authentication data measured by the external device, and wherein the internal computing unit is further configured to authenticate the connection if the difference value is less than a predetermined threshold difference value, and to not authenticate the connection if the difference value equals or exceeds the predetermined threshold difference value.

According to embodiments of the sixth part of aspect 256SE the communication between the implant and the external device is a wireless communication.

According to embodiments of the sixth part of aspect 256SE the communication between the implant and the external device is a conductive communication.

According to embodiments of the sixth part of aspect 256SE the communication between the implant and the external device is a conductive communication adapted to transport the input authentication data to the implant.

According to embodiments of the sixth part of aspect 256SE the communication between the implant and the external device is a conductive communication adapted to transport the authentication data to the external device.

In a seventh part of aspect 256SE there is provided a method of authenticating a connection between an implant implanted in a patient, and an external device, the method comprising:
a. using a sensation generated by the body and detectable by the implant and the external device,
b. storing, by the implant, authentication data, related to the sensation,
c. providing to the implant input from the external device about the sensation, resulting in input authentication data, and
d. authenticating the connection based on an analysis of the input authentication data and the authentication data.

### Aspect 257SE Device Synchronization Sensation Unit—Sensation Unit for Authenticating a Connection Between an Implant and the External Device—Embodiments of Aspect 257SE of the Disclosure

In a first part of aspect 257SE, a method of authenticating a connection between an implant, implanted in a patient, and an external device is provided. The method comprises generating, by a sensation generator of the implant, a sensation detectable by a sense of the patient. The method further comprises storing, by the implant, authentication data, related to the generated sensation. The method further comprises providing, by the patient, input to the external device, resulting in input authentication data. The method further comprises authenticating the connection based on a comparison of the input authentication data and the authentication data.

Similarly, to the first part of aspect 248SE, a main advantage of this way of authenticating a connection is that only the patient may be able to experience the sensation. Thus, only the patient may be able to authenticate the connection by providing authentication input corresponding to the sensation generation.

According to some embodiments of the first part of aspect 257SE the method further comprises the step of communicating the authentication data from the sensation generator to the implant using a wireless communication.

According to some embodiments of the first part of aspect 257SE the method further comprises the step of communicating the authentication data from the sensation generator to the implant using a wired communication.

According to some embodiments of the first part of aspect 257SE the method further comprises the step of communicating further data between the implant and the external device following positive authentication.

According to some embodiments of the first part of aspect 257SE the authentication data comprises a timestamp of the sensation and wherein the input authentication data comprises a timestamp of the input from the patient

According to some embodiments of the first part of aspect 257SE the step of authenticating the connection comprises calculating a time difference between the timestamp of the sensation and the timestamp of the input from the patient, and upon determining that the time difference is less than a threshold, authenticating the connection.

According to some embodiments of the first part of aspect 257SE the authentication data comprises a number of times that the sensation is generated by the sensation generator, and wherein the input authentication data comprises an input from the patient relating to a number of times the patient detected the sensation. Authenticating the connection may then comprise upon determining that the number of times of the authentication data and the input authentication data are equal, authenticating the connection.

According to some embodiments of the first part of aspect 257SE the sensation may comprise a plurality of sensation components.

According to some embodiments of the first part of aspect 257SE the sensation or sensation components may comprise a vibration.

According to some embodiments of the first part of aspect 257SE the sensation or sensation components may comprise a sound.

According to some embodiments of the first part of aspect 257SE the sensation or sensation components may comprise a photonic signal.

According to some embodiments of the first part of aspect 257SE the sensation or sensation components may comprise a light signal.

According to some embodiments of the first part of aspect 257SE the sensation or sensation components may comprise an electric signal.

According to some embodiments of the first part of aspect 257SE the sensation or sensation components may comprise a heat signal.

According to some embodiments of the first part of aspect 257SE the communication between the implant and the external device may be a wireless communication.

According to some embodiments of the first part of aspect 257SE the communication between the implant and the external device may be a conductive communication.

According to some embodiments of the first part of aspect 257SE the method further comprises the step of transmitting the input authentication data from the external device to the implant, wherein the comparison is performed by the implant.

According to some embodiments of the first part of aspect 257SE the method further comprises the step of transmitting the authentication data from the implant to the external device, wherein the comparison is performed by the external device.

According to some embodiments of the first part of aspect 257SE the comparison is performed by the implant and the method further comprises the step of continuously requesting by the external device, or receiving at the external device, information of an authentication status of the connection between the implant and the external device. The method may further comprise upon determining, at the external device, that the connection is authenticated, transmitting further data from the external device to the implant.

According to some embodiments of the first part of aspect 257SE the further data comprises at least data for updating a control program running in the implant, or operation instructions for operating the implant.

According to some embodiments of the first part of aspect 257SE the comparison is performed by the external device and the method further comprises the step of continuously requesting by the implant, or receiving at the implant, information of an authentication status of the connection between the implant and the external device. The method may further comprise upon determining, at the implant, that the connection is authenticated, transmitting further data from the implant to the external device.

According to some embodiments of the first part of aspect 257SE the further data comprises data sensed by a sensor connected to the implant.

According to some embodiments of the first part of aspect 257SE the sensation generator is adapted to be implanted in the patient.

According to some embodiments of the first part of aspect 257SE the sensation generator is configured to be worn in contact with the skin of the patient.

According to some embodiments of the first part of aspect 257SE the sensation generator is configured generate the sensation without being in physical contact with the patient.

In a second part of aspect 257SE, an implant, implanted in a patient and adapted for connection with an external device is provided. The implant may comprise a sensation generator. The implant may be configured for receiving authentication data related to a sensation generated by the sensation generator from the sensation generator. The implant may be further configured for storing the authentication data. The implant may be further configured for receiving input authentication data from the external device. The implant may comprise an internal communication unit. The internal communication unit may be configured for comparing the authentication data to the input authentication data. The internal communication unit may be further configured for performing authentication of the connection based on the comparison.

According to some embodiments of the second part of aspect 257SE the implant may further comprise a wireless communication system configured for receiving the authentication data from the sensation generator.

According to some embodiments of the second part of aspect 257SE the implant may further comprise a wired transceiver configured for receiving the authentication data from the sensation generator.

According to some embodiments of the second part of aspect 257SE the implant may further comprise a wired transceiver configured for receiving the authentication data from the sensation generator.

According to some embodiments of the second part of aspect 257SE the implant may further be configured for communicating further data to the external device following positive authentication.

According to some embodiments of the second part of aspect 257SE the authentication data comprises a timestamp of the sensation and wherein the input authentication data comprises a timestamp of the input from the patient.

According to some embodiments of the second part of aspect 257SE authenticating the connection comprises calculating a time difference between the timestamp of the sensation and the timestamp of the input from the patient, and upon determining that the time difference is less than a threshold, authenticating the connection.

According to some embodiments of the second part of aspect 257SE the authentication data may comprise a number of times that the sensation is generated by the sensation generator. The input authentication data may comprise an input from the patient relating to a number of times the patient detected the sensation. Authenticating the connection may comprise upon determining that the number of times of the authentication data and the input authentication data are equal, authenticating the connection.

According to some embodiments of the second part of aspect 257SE the communication between the implant and the external device is a wireless communication.

According to some embodiments of the second part of aspect 257SE the communication between the implant and the external device is a conductive communication.

In a third part of aspect 257SE, a sensation generator, adapted to generate a sensation detectable by a sense of the patient, is provided. The sensation generator being configured to, upon request, generate the sensation and transmit authentication data, related to the generated sensation, to an implant, when implanted in a patient.

According to some embodiments of the third part of aspect 257SE the sensation generator may further be configured to transmit the authentication data to the implant using wireless communication.

According to some embodiments of the third part of aspect 257SE the sensation generator may further be configured to transmit the authentication data to the implant using wired communication.

According to some embodiments of the third part of aspect 257SE the sensation generator is further configured to receive the request from the implant.

According to some embodiments of the third part of aspect 257SE the sensation generator is further configured to receive the request from an external device.

According to some embodiments of the third part of aspect 257SE the sensation generator is further configured to create the sensation comprising a plurality of sensation components.

According to some embodiments of the third part of aspect 257SE the sensation generator is further configured to create the sensation or sensation components by vibration of the sensation generator.

According to some embodiments of the third part of aspect 257SE the sensation generator is further configured to create the sensation or sensation components by producing a sound.

According to some embodiments of the third part of aspect 257SE the sensation generator is further configured to create the sensation or sensation components by providing a photonic signal.

According to some embodiments of the third part of aspect 257SE the sensation generator is further configured to create the sensation or sensation components by providing a light signal.

According to some embodiments of the third part of aspect 257SE the sensation generator is further configured to create the sensation or sensation components by providing an electric signal.

According to some embodiments of the third part of aspect 257SE the sensation generator is further configured to create the sensation or sensation components by providing a heat signal.

According to some embodiments of the third part of aspect 257SE the sensation generator is adapted to be implanted in the patient.

According to some embodiments of the third part of aspect 257SE the sensation generator is further configured to be worn in contact with the skin of the patient.

According to some embodiments of the third part of aspect 257SE the sensation generator is further configured to generate the sensation without being in physical contact with the patient.

In a fourth part of aspect 257SE, there is provided a system comprising a sensation generator according to the third part of aspect 257SE, an implant according to the second part of aspect 257SE and an external device. The system may be configured for performing methods according to the first part of aspect 257SE.

According to embodiments of the second part of, or other parts of, aspect 257SE the implant comprises at least one of:
a pacemaker unit, or an implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

In a fifth part of aspect 257SE there is provided a computer program product comprising a computer-readable storage medium with instructions adapted to carry out at least parts of any one of the embodiments of aspect 257SE, when executed by a the implant or external device having processing capability.

According to embodiments of the second part of, or other parts of, aspect 257SE the implant comprises an internal control unit adapted to be involved in at least a part of the actions performed by the implant in at least a part of any one of the embodiments of aspect 257SE.

According to embodiments of the second part of, or other parts of, aspect 257SE the implant comprises an internal computing unit adapted to be involved in at least a part of the actions performed by the implant in at least a part of any one of the other embodiments of aspect 257SE, or
wherein the internal computing unit is adapted to be involved in at least a part of the actions performed by the implant in at least a part of any one of the other embodiments of aspect 257SE.

### Aspect 258SE Device Synchronization Sensation—Authenticating a Connection Between an Implant and the External Device by Using Sensations—Embodiments of Aspect 258SE of the Disclosure

In a first part of aspect 258SE, there is provided a method of authenticating a connection between an implant implanted in a patient, and an external device. The method comprising:
a. using a sensation generated by a body of the patient or a sensation generator, the sensation being detectable by the implant and the external device,
b. storing, by the implant and by the external device authentication data, related to the sensation,
c. providing at least one of; input from the external device to the implant and input from the implant to the external about the sensation, resulting in input authentication data, and
d. authenticating the connection based on an analysis of the input authentication data and the authentication data.

According to some embodiments of the first part of aspect 258SE the method further comprises the step of communicating further data between the implant and the external device following positive authentication.

According to some embodiments of the first part of aspect 258SE the authentication data comprises a characteristic of the sensation, wherein the input authentication data comprises a second characteristic of the sensation, and wherein authenticating the connection comprises: comparing the characteristic of the sensation with the second characteristic of the sensation.

According to some embodiments of the first part of aspect 258SE the authentication data comprises a timestamp of the sensation, wherein the input authentication data comprises a second timestamp of the sensation, and wherein authenticating the connection comprises: comparing the time stamp of the sensation with the second timestamp of the sensation.

According to some embodiments of the first part of aspect 258SE the authentication data comprises a number of times that the sensation is generated by the sensation generator, and wherein the input authentication data comprises an input from the patient relating to a number of times the patient detected the sensation to be stored in the external device, wherein authenticating the connection comprises: upon determining that the number of times of the authentication data and the input authentication data are equal, authenticating the connection.

According to some embodiments of the first part of aspect 258SE the sensation comprises a plurality of sensation components.

According to some embodiments of the first part of aspect 258SE the sensation or sensation components comprise a vibration.

According to some embodiments of the first part of aspect 258SE the sensation or sensation components comprise a sound.

According to some embodiments of the first part of aspect 258SE the sensation or sensation components comprise a photonic signal.

According to some embodiments of the first part of aspect 258SE the sensation or sensation components comprise a light signal.

According to some embodiments of the first part of aspect 258SE the sensation or sensation components comprise an electric signal.

According to some embodiments of the first part of aspect 258SE the sensation or sensation components comprise a heat signal.

According to some embodiments of the first part of aspect 258SE the sensation generator is contained within the implant.

According to some embodiments of the first part of aspect 258SE the communication between the implant and the external device is a wireless communication or a conductive communication.

According to some embodiments of the first part of aspect 258SE the communication between the implant and the external device is both a wireless communication and a conductive communication.

According to some embodiments of the first part of aspect 258SE the method further comprises the step of:
transmitting the input authentication data from the external device to the implant,
wherein the analysis is performed by the implant.

According to some embodiments of the first part of aspect 258SE the method further comprises the step of:
transmitting the authentication data from the implant to the external device,
wherein the analysis is performed by the external device.

According to some embodiments of the first part of aspect 258SE the implant comprises a motor for controlling a physical function in the body of the patient, wherein the motor being the sensation generator.

According to some embodiments of the first part of aspect 258SE the sensation is a vibration created by running the motor.

According to some embodiments of the first part of aspect 258SE the sensation is a sound created by running the motor.

According to some embodiments of the first part of aspect 258SE the analysis is performed by the implant, the method further comprising the step of:
continuously requesting by the external device, or receiving at the external device, information of an authentication status of the connection between the implant and the external device, and upon determining, at the external device, that the connection is authenticated, transmitting further data from the external device to the implant.

According to some embodiments of the first part of aspect 258SE the further data comprises at least one of:
a. data for updating a control program running in the implant, and
b. operation instructions for operating the implant.

According to some embodiments of the first part of aspect 258SE the analysis is performed by the external device, the method further comprising the step of: continuously requesting by the implant, or receiving at the implant, information of an authentication status of the connection between the implant and the external device, and upon determining, at the implant, that the connection is authenticated, transmitting further data from the implant to the external device.

According to some embodiments of the first part of aspect 258SE the further data comprises data sensed by a sensor connected to the implant.

In a second part of aspect 258SE, there is provided an implant, implanted in a patient, adapted for connection with an external device, the implant connected to a sensation generator or a sensor for recording a sensation generated by the body of a the patient, the implant being configured for:
a. storing authentication data, related to a sensation generated by the sensation generator or by the body of the patient,
b. receiving input authentication data from the external device, and wherein the implant comprises an internal computing unit configured for:
   i. analyzing the authentication data and the input authentication data, and
   ii. performing authentication of the connection based on the analysis.

According to some embodiments of the second part of aspect 258SE the implant is further configured for communicating further data to the external device following positive authentication.

According to some embodiments of the second part of aspect 258SE the authentication data comprises a characteristic of the sensation, wherein the input authentication data comprises a second characteristic of the sensation, and wherein authenticating the connection comprises: comparing the characteristic of the sensation with the second characteristic of the sensation.

According to some embodiments of the second part of aspect 258SE authentication data comprises a timestamp of the sensation, wherein the input authentication data comprises a second timestamp of the sensation, and wherein authenticating the connection comprises: comparing the time stamp of the sensation with the second timestamp of the sensation.

According to some embodiments of the second part of aspect 258SE the authentication data comprises a number of times that the sensation is generated by the sensation generator, and wherein the input authentication data comprises an input from the patient relating to a number of times the patient detected the sensation to be stored in the external device, wherein authenticating the connection comprises: upon determining that the number of times of the authentication data and the input authentication data are equal, authenticating the connection.

According to some embodiments of the second part of aspect 258SE the sensation generator is contained within the implant.

According to some embodiments of the second part of aspect 258SE the sensation generator is configured to create the sensation comprising a plurality of sensation components.

According to some embodiments of the second part of aspect 258SE the sensation generator is configured to create the sensation or sensation components by vibration of the sensation generator.

According to some embodiments of the second part of aspect 258SE the sensation generator is configured to create the sensation or sensation components by playing a sound.

According to some embodiments of the second part of aspect 258SE the sensation generator is configured to create the sensation or sensation components by providing a photonic signal.

According to some embodiments of the second part of aspect 258SE the sensation generator is configured to create the sensation or sensation components by providing a light signal.

According to some embodiments of the second part of aspect 258SE the sensation generator is configured to create the sensation or sensation components by providing an electric signal.

According to some embodiments of the second part of aspect 258SE the sensation generator is configured to create the sensation or sensation components by providing a heat signal.

According to some embodiments of the second part of aspect 258SE the communication between the implant and the external device is both a wireless communication and a conductive communication.

According to some embodiments of the second part of aspect 258SE the implant comprises a motor for controlling a physical function in the body of the patient, wherein the motor being the sensation generator.

According to some embodiments of the second part of aspect 258SE the implant comprises a motor for controlling a physical function in the body of the patient, wherein the motor being the sensation generator.

According to some embodiments of the second part of aspect 258SE the sensation is a vibration created by running the motor.

According to some embodiments of the second part of aspect 258SE the sensation is a sound created by running the motor.

In a third part of aspect 258SE, there is provided an external device, adapted for connection with an implant, implanted in a patient, the external device comprising:
a. an interface for receiving, by the patient, input to the external device, resulting in input authentication data,
b. a receiver for receiving authentication data from the implant, the authentication data relating to a generated sensation of a sensation generator connected to the implant or to a measured sensation generated by a body of the patient;
c. an external computing unit configured for:
   i. analyzing the authentication data and the input authentication data, and
   ii. performing authentication of the connection based on the analysis.

According to some embodiments of the third part of aspect 258SE the external device is further configured for communicating further data to the implant following positive authentication.

According to some embodiments of the third part of aspect 258SE the authentication data comprises a characteristic of the sensation, wherein the input authentication data comprises a second characteristic of the sensation, and wherein authenticating the connection comprises: comparing the characteristic of the sensation with the second characteristic of the sensation.

According to some embodiments of the third part of aspect 258SE authentication data comprises a timestamp of the sensation, wherein the input authentication data comprises a second timestamp of the sensation, and wherein authenticating the connection comprises: comparing the time stamp of the sensation with the second timestamp of the sensation.

According to some embodiments of the third part of aspect 258SE the authentication data comprises a number of times that the sensation is generated by the sensation generator, and wherein the input authentication data comprises an input from the patient relating to a number of times the patient detected the sensation to be stored in the external device, wherein authenticating the connection comprises: upon determining that the number of times of the authentication data and the input authentication data are equal, authenticating the connection.

According to some embodiments of the third part of aspect 258SE the communication between the implant and the external device is a wireless communication or a conductive communication.

According to some embodiments of the third part of aspect 258SE the communication between the implant and the external device is both a wireless communication and a conductive communication.

According to some embodiments of the third part of aspect 258SE the external device further comprises a conductive member configured to be in electrical connection with the external device, wherein the conductive member is configured to be placed in electrical connection with a skin of the patient for conductive communication with the implant.

According to some embodiments of the first part of aspect 258SE the method further comprises transmitting further data between the implant and the external device, wherein the further data is used or acted upon, only after authentication of the connection is performed.

According to some embodiments of the second part of aspect 258SE the implant comprises at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

In a fourth part of aspect 258SE there is provided a computer program product of, or adapted to be run on, an external device, adapted for connection with an implant, implanted in a patient, the external device comprising:
a. an interface for receiving, by the patient, input to the external device, resulting in input authentication data,
b. a receiver for receiving authentication data from the implant, the authentication data relating to a generated sensation of a sensation generator or to a measured sensation generated by a body of the patient, the receiver being part of the implant or external device,
c. an external computing unit,
wherein the computer program product is configured to cause the external computing unit to:
   i. analyze the authentication data and the input authentication data, and
   ii. perform authentication of the connection based on the analysis.

According to some embodiments of the fourth part of aspect 258SE the computer program product is configured to cause the external device to communicate further data to the implant following positive authentication.

According to some embodiments of the fourth part of aspect 258SE the authentication data comprises a characteristic of the sensation, wherein the input authentication data comprises a second characteristic of the sensation, and wherein authenticating the connection comprises: comparing the characteristic of the sensation with the second characteristic of the sensation.

According to some embodiments of the fourth part of aspect 258SE authentication data comprises a timestamp of the sensation, wherein the input authentication data comprises a second timestamp of the sensation, and wherein authenticating the connection comprises: comparing the time stamp of the sensation with the second timestamp of the sensation.

According to some embodiments of the fourth part of aspect 258SE the authentication data comprises a number of times that the sensation is generated by the sensation generator, and wherein the input authentication data comprises an input from the patient relating to a number of times the patient detected the sensation to be stored in the external device, wherein authenticating the connection comprises: upon determining that the number of times of the authentication data and the input authentication data are equal, authenticating the connection.

According to some embodiments of the fourth part of aspect 258SE the communication between the implant and the external device is a wireless communication or a conductive communication.

According to some embodiments of the fourth part of aspect 258SE the communication between the implant and the external device is both a wireless communication and a conductive communication.

In a fifth part of aspect 258SE there is provided a computer program product adapted to be run on, an implant, implanted in a patient, adapted for connection with an external device, the implant comprising:
a. an interface for receiving, by the patient, input to the implant, resulting in input authentication data,
b. a receiver for receiving authentication data from the external device, the authentication data relating to a generated of a sensation generator of the implant or the external device or to a measured sensation generated by a body of the patient,
c. a computing unit, wherein the computer program product is configured to cause the computing unit to:
   i. analyze the authentication data and the input authentication data, and
   ii. perform authentication of the connection based on the analysis.

According to some embodiments of the fifth part of aspect 258SE the computer program product is configured to cause the implant to accept further communication with further data received by the implant following positive authentication.

According to some embodiments of the fifth part of aspect 258SE the authentication data comprises a characteristic of the sensation, wherein the input authentication data comprises a second characteristic of the sensation, and wherein authenticating the connection comprises: comparing the characteristic of the sensation with the second characteristic of the sensation.

According to some embodiments of the fifth part of aspect 258SE authentication data comprises a timestamp of the sensation, wherein the input authentication data comprises a second timestamp of the sensation, and wherein authenticating the connection comprises: comparing the time stamp of the sensation with the second timestamp of the sensation.

According to some embodiments of the fifth part of aspect 258SE the authentication data comprises a number of times that the sensation is generated by the sensation generator, and wherein the input authentication data comprises an input from the patient relating to a number of times the patient detected the sensation to be stored in the external device, wherein authenticating the connection comprises: upon determining that the number.

According to some embodiments of the fifth part of aspect 258SE the further communication between the implant and the external device is a wireless communication.

According to some embodiments of the fifth part of aspect 258SE the communication between the implant and the external device is a wireless communication.

According to some embodiments of the fifth part of aspect 258SE the communication between the implant and the external device is a wireless communication or a conductive communication.

According to some embodiments of the fifth part of aspect 258SE the communication between the implant and the external device is both a wireless communication and a conductive communication.

The implant according to the second part of aspect 258SE and/or with ability to use any of the methods of the first part of aspect 258SE, and/or with ability to perform the authentication process in any of third part of aspect 258SE and/or with ability to use any of the computer program products of the fourth part of aspect 258SE, may comprise an internal control unit adapted to be involved in at least a part of the actions performed by the implant in at least a part of any one of the embodiments of the above.

The embodiments of aspect 258SE may have close similarities with the embodiments of aspect 248SE. When referring to one or the other within this document, it should be understood that both may be considered for reference.

### Aspect 307SE Communication Remote Control—Remote Wake Signal—Embodiments of Aspect 307SE of the Disclosure

According to a first part of aspect 307SE, a system for controlling a medical implant implanted in a patient is provided. The system comprises an internal control unit adapted to be arranged within the patient's body and communicatively coupled to the medical implant. The internal control unit may comprise a processing unit having a sleep mode and an active mode, and a sensor configured to detect a wake signal. The system further comprises an external control unit adapted to be arranged outside of the patient's body, the external control unit comprises a signal provider configured to provide the wake signal, wherein the internal control unit is further configured to set the processing unit to the active mode in response to the sensor detecting the wake signal.

According to some embodiments of the first part of aspect 307SE, the signal provider is an acoustic source configured to provide an acoustic signal as the wake signal.

According to some embodiments of the first part of aspect 307SE, the signal provider is a magnetic source configured to provide a magnetic signal as the wake signal.

According to some embodiments of the first part of aspect 307SE, the sensor is configured to detect the received signal strength of a signal, and the internal control unit is further configured to set the processing unit to the active mode in response to the sensor detecting a signal exceeding a threshold signal strength.

According to some embodiments of the first part of aspect 307SE, the sensor is configured to provide a control signal indicative of a wake signal, the internal control unit is configured to set the processing unit to the active mode in response to the control signal, and the internal control unit is configured to control a supply of energy to the processing unit in response to the control signal.

According to some embodiments of the first part of aspect 307SE, the wake signal comprises a predetermined signal pattern, and the internal control unit is further configured to set the processing unit to the active mode in response to the sensor detecting the predetermined signal pattern.

According to some embodiments of the first part of aspect 307SE, the magnetic source comprises a first coil.

According to some embodiments of the first part of aspect 307SE, the magnetic source further comprises a second coil arranged perpendicular to the first coil, whereby to collectively provide a substantially even magnetic field.

According to some embodiments of the first part of aspect 307SE, the first coil and/or the second coil is configured to provide a signal as a magnetic field with a frequency of 9 to 315 kilohertz, kHz. The frequency may be less than or equal to 125 kHz, preferably less than 58 kHz. In some examples, the frequency is less than 50 kHz, preferably less than 20 kHz, more preferably less than 10 kHz.

According to some embodiments of the first part of aspect 307SE, the magnetic source comprises a magnet. The magnet may, in some examples, be a permanent magnet.

According to some embodiments of the first part of aspect 307SE, the magnetic source has an off state in which the magnetic source does provides a magnetic field and an on state in which the magnetic source provides a magnetic field. The magnetic source may, in some examples, further comprises a shielding means for preventing, when the magnetic source is in the off state, the magnetic source from providing a magnetic field.

According to some embodiments of the first part of aspect 307SE, the sensor comprises a hall effect sensor, a fluxgate sensor, an ultra-sensitive magnetic field sensor or a magneto-resistive sensor. In some examples, the sensor may comprise a third coil having an iron core.

According to some embodiments of the first part of aspect 307SE, the internal control unit comprises a first communication unit for receiving and/or transmitting data from and/or to the external control unit, and the external control unit comprises a second communication unit for transmitting and/or receiving data to and/or from the internal control unit.

According to some embodiments of the first part of aspect 307SE, the sensor may be comprised in the first communication unit.

According to some embodiments of the first part of aspect 307SE, the system further comprises a frequency detector, communicatively coupled to the internal control unit and configured to detect a frequency for data communication between the first communication unit and the second communication unit. The frequency detector may comprise an antenna.

According to some embodiments of the first part of aspect 307SE, when the system comprises a first communication unit and a second communication unit, the first communication unit and the second communication unit may be configured for data communication using magnetic induction via the first coil.

According to some embodiments of the first part of aspect 307SE, the first communication unit comprises a high-sensitivity magnetic field detector.

According to some embodiments of the first part of aspect 307SE, the first communication unit comprises a fourth coil for communicating with the second communication unit via the first coil.

According to some embodiments of the first part of aspect 307SE, the system further comprises an implantable energy source electrically connected to the first communication unit, wherein the implantable energy source is adapted to be charged by the external control unit via the first communication unit.

According to some embodiments of the first part of aspect 307SE, the implantable energy source is configured to be charged via magnetic induction between the first coil and the fourth coil.

According to some embodiments of the first part of aspect 307SE, the internal control unit is configured to control the charging of the implantable energy source by controlling a receipt of electrical power from the external control unit at the first communication unit.

According to some embodiments of the first part of aspect 307SE, the internal control unit is further configured to control the charging of the implantable energy source by controlling a transmission of electrical power from the external control unit to the first communication unit.

According to some embodiments of the first part of aspect 307SE, the system further comprises a sensation generator configured to generate a sensation detectable by a sense of the patient, the sensation generator being communicatively coupled to the internal control unit or the external control unit and being configured to, upon request, generate the sensation when the medical implant is implanted in the patient. In some examples, the sensation generator is configured to receive the request from the internal control unit of the medical implant. The sensation generator may be configured to receive the request from an external device. In some embodiments, the generated sensation may comprise a plurality of sensation components. The sensation generator may be configured to create the sensation or sensation components by at least one of a vibration of the sensation generator; producing a sound; providing a photonic signal; providing a light signal; providing an electric signal; and a heat signal. The sensation generator may be configured to be implanted in the patient, and/or be configured to be worn in contact with the skin of the patient. In some examples, the sensation generator is configured generate the sensation without being in physical contact with the patient.

According to some embodiments of the first part of aspect 307SE, the external control unit comprises a wireless remote control.

According to some embodiments of the first part of aspect 307SE, the wireless remote control comprises an external signal transmitter, and the internal control unit is further configured to receive one or more control signals transmitted by the external signal transmitter and to control an operation of the medical implant based at least in part on said signal, when the processing unit is in the active state.

According to some embodiments of the first part of aspect 307SE, the one or more control signals is selected from the group consisting of:
a sound signal;
an ultrasound signal;
an electromagnetic signal;
an infrared signal;
a visible light signal;
an ultraviolet light signal;
a laser signal;
a microwave signal;
a radio wave signal;
an X-ray radiation signal; and
a gamma radiation signal.

According to a second part of aspect 307SE, a method for controlling an implant implanted in a patient is provided. The method comprises:
monitoring for signals by a sensor comprised in an internal control unit communicatively coupled to the medical implant;
providing, from a signal provider comprised in an external control unit, a wake signal, the external control unit being adapted to be arranged outside of the patient's body;
setting, by the internal control unit and in response to a detected wake signal, a mode of a processing unit comprised in the internal control unit from a sleep mode to an active mode.

According to some embodiments of the second part of aspect 307SE, the method further comprises:
detecting, using a frequency detector, a frequency for data communication between a first communication unit and a second communication unit, the first communication unit being associated with the internal control unit and the second communication unit being associated with the external control unit,
wherein the frequency detector is communicatively coupled to the internal control unit.

According to some embodiments of the second part of aspect 307SE, the method further comprises:
determining, using the frequency detector, the frequency for data communication; and
initiating data communication between the first communication unit and the second communication unit.

According to some embodiments of the second part of aspect 307SE, the data communication comprises one or more control instructions for controlling the medical implant.

According to some embodiments of the second part of aspect 307SE, the method further comprises generating, using a sensation generator communicatively coupled to the internal control unit, a sensation detectable by a sense of the patient.

According to some embodiments of the second part of aspect 307SE, the data communications further comprise a request to generate the sensation.

According to some embodiments of the second part of aspect 307SE, the sensation is generated in response to a sensor measurement from the implant.

According to a third part of aspect 307SE, an implant is provided. The implant comprises a control unit, and the control unit comprises a processing unit having a sleep mode and an active mode; and a sensor configured to detect a wake signal. The control unit is configured to set the processing unit to the active mode in response to the sensor detecting the wake signal.

According to some embodiments of the third part of aspect 307SE, the sensor is a piezoelectric sensor for detecting acoustic signals.

According to some embodiments of the second part of aspect 307SE, the sensor is a magnetic sensor for detecting magnetic signals.

According to some embodiments of the second part of aspect 307SE, the sensor is configured to detect the received signal strength of a signal.

According to some embodiments of the second part of aspect 307SE, the control unit is further configured to set the processing unit to the active mode in response to the sensor detecting a signal exceeding a threshold signal strength.

According to some embodiments of the second part of aspect 307SE, the wake signal comprises a predetermined signal pattern, and the control unit is further configured to set the processing unit to the active mode in response to the sensor detecting the predetermined signal pattern.

According to some embodiments of the second part of aspect 307SE, the sensor is a hall effect sensor, a fluxgate sensor, an ultra-sensitive magnetic field sensor or a magneto-resistive sensor.

According to some embodiments of the second part of aspect 307SE, the sensor comprises a first coil.

According to some embodiments of the second part of aspect 307SE, the implant further comprises a communication unit for data communication.

According to some embodiments of the second part of aspect 307SE, the sensor is comprised in the first communication unit.

According to some embodiments of the second part of aspect 307SE, the implant further comprises a frequency detector, communicatively coupled to the control unit and configured to detect a frequency for the data communication. The frequency detector may comprise an antenna.

According to some embodiments of the second part of aspect 307SE, the communication unit comprises a high-sensitivity magnetic field detector.

According to some embodiments of the second part of aspect 307SE, the communication unit comprises a fourth coil for communicating with an external communication unit.

According to some embodiments of the second part of aspect 307SE, the implant further comprises an implantable energy source electrically connected to the communication unit, wherein the implantable energy source is adapted to be wirelessly charged by an external charging unit.

According to some embodiments of the second part of aspect 307SE, the implantable energy source is configured to be charged via magnetic induction of the first coil.

According to some embodiments of the second part of aspect 307SE, the implantable energy source is configured to be charged via piezoelectric operation of the piezoelectric sensor.

According to some embodiments of the second part of aspect 307SE, the internal control unit is configured to control the charging of the implantable energy source by controlling a receipt of electrical power at the communication unit.

According to some embodiments of the second part of aspect 307SE, the implant further comprises a sensation generator configured to generate a sensation detectable by a sense of the patient, the sensation generator being communicatively coupled to the control unit and being configured to, upon request, generate the sensation when the medical implant is implanted in the patient.

According to some embodiments of the second part of aspect 307SE, the sensation generator is configured to receive the request from the control unit of the medical implant.

According to some embodiments of the second part of aspect 307SE, the sensation generator is configured to receive the request via the communication unit.

According to some embodiments of the second part of aspect 307SE, the generated sensation comprises a plurality of sensation components.

According to some embodiments of the second part of aspect 307SE, the sensation generator may be configured to create the sensation or sensation components by at least one of:
a vibration of the sensation generator;
producing a sound;
providing a photonic signal;
providing a light signal;
providing an electric signal; and
a heat signal.

According to some embodiments of the second part of aspect 307SE, the piezoelectric sensor is the sensation generator or is comprised in the sensation generator, and the sensation or a sensation component comprises a vibration of the sensation generator or producing a sound, and the vibration of the sensation generator or the production of the sound is generated by electric stimulation of the piezoelectric sensor.

The implant according to the third part of aspect 307SE or the system according to the first part of aspect 307SE, and/or with ability to use any of the methods of the second part of aspect 307SE, and/or with ability to perform the authentication process in any of third part of aspect 258SE, may comprise an internal control unit adapted to be involved in at least a part of the actions performed by the implant in at least a part of any one of the embodiments of the above.

The embodiments of aspect 307SE may have close similarities with the embodiments of aspect 315SE. When referring to one or the other within this document, it should be understood that both may be considered for reference.

### Aspect 308SE Energy Power-Supply Capacitor—Energy Burst Provider—Embodiments of Aspect 308SE of the Disclosure

According to a first part of aspect 308SE, an apparatus for powering an implant for a human patient is provided. The apparatus comprises an implantable energy source for providing energy to the implant, an energy provider connected to the implantable energy source and connected to an energy consuming part of the implant, the energy provider being configured to store energy to provide a burst of energy to the energy consuming part, wherein the energy provider is configured to be charged by the implantable energy source and to provide the energy consuming part with electrical power during startup of the energy consuming part.

According to some embodiments, the discharging from the implantable energy source of the energy consuming part is slower than the energy needed for the energy consuming part.

According to some embodiments, the discharging from the implantable energy source during startup of the energy consuming part is slower than the energy needed for startup of the energy consuming part.

According to some embodiments, a maximum energy consumption of the energy consuming part is higher than the maximum energy capable of being delivered by the implantable energy source without causing damage to the implantable energy source, and wherein the energy provider is adapted to deliver energy to the energy consuming part the energy consuming part.

According to some embodiments, a maximum energy consumption of the energy consuming part is higher than the maximum energy capable of being delivered by the implantable energy source without causing damage to the implantable energy source, and wherein the energy provider is adapted to deliver an energy burst corresponding to difference between the required energy consumption and the maximum energy capable of being delivered by the implantable energy source.

According to some embodiments, the implantable energy source is a re-chargeable battery.

According to some embodiments, the implantable energy source is a solid-state battery.

According to some embodiments, the battery is a trionychoid battery.

According to some embodiments, the implantable energy source is connected to the energy consuming part and configured to power the energy consuming part after it has been started using the energy provider.

According to some embodiments, the energy provider is a capacitor.

According to some embodiments, the energy provider is a start capacitor.

According to some embodiments, the energy provider is a run capacitor.

According to some embodiments, the energy provider is a dual run capacitor.

According to some embodiments, the apparatus further comprises a second energy provider configured to be charged by the implantable energy source and to provide the energy consuming part with electrical power.

According to some embodiments, the energy provider is a supercapacitor.

According to some embodiments, the energy consuming part is a motor for operating a device or function of the implant.

According to some embodiments, the energy consuming part is at least one of
a device for providing electrical stimulation to a tissue portion of the body of the patient,
a CPU for encrypting information
a transmitting and/or receiving unit for communication with an external unit
a measurement unit or a sensor
a data collection unit
a solenoid
a piezo-electrical element
a memory metal unit.

According to some embodiments, the energy consuming part is motor for powering a hydraulic pump.

According to some embodiments, the energy consuming part is a feedback unit.

According to some embodiments, the feedback unit is a vibrator.

According to some embodiments, the energy consuming part is configured to operate a valve comprised in the implant.

According to some embodiments, the energy consuming part is a control unit for controlling at least a part of the implant.

According to some embodiments, the control unit has a sleep mode and an operational mode, wherein the apparatus at least is configured to provide the control unit with electrical power for transitioning from the sleep mode to the operational mode.

According to some embodiments, the apparatus is further comprising:
an external energy source configured be arranged outside of the patient's body and configured to provide energy to the implantable energy source,
an implantable charger configured to be electrically connected to the implantable energy source and enable charging of the implantable energy source by the external energy source.

According to some embodiments, the charger is configured to control the charging of the implantable energy source by controlling a receipt of electrical power from the external energy source at the implantable charger.

According to some embodiments, the internal charger is configured to control the charging of the implantable energy source by controlling a transmission of electrical power from the external energy source to the implantable charger.

According to some embodiments, the apparatus is further comprising an energy source indicator, wherein the energy source indicator is further configured to indicate a functional status of the implantable energy source.

According to some embodiments, the functional status indicates at least one of charge level and temperature of the implantable energy source.

According to some embodiments, the controller is further configured to include the functional status in a signal transmitted to the outside of the body.

According to some embodiments, the charger comprises an electromagnetic coil configured to receive electrical power wirelessly from the external energy source.

According to some embodiments, the implantable charger or the external energy source is configured to receive the functional status from the energy source indicator and control the charging of the implantable energy source based on the functional status.

According to a part of aspect 308SE, an apparatus for powering an implant for a human patient is provided. The apparatus comprises a first implantable energy source for providing energy to an energy consuming part of the implant, a second implantable energy source connected to the implantable energy source and connected to the energy consuming part, wherein the second implantable energy source is configured to be charged by the implantable energy source and to provide the energy consuming part with electrical power during startup of the energy consuming part, wherein the second implantable energy source has a higher energy density than the first implantable energy source.

According to some embodiments, the second implantable energy source has a higher maximum energy output per time unit.

According to some embodiments, the first implantable energy source is a non-chargeable battery, and wherein the second implantable energy source is a chargeable energy storage.

According to a third part of aspect 308SE, a method for powering an implant for a human patient is provided. The method comprising the steps of:
initiating an energy consuming part of the implant, the energy consuming part being connected to an implantable energy source;
providing an initial burst of energy to the energy consuming part using an energy provider connected to the implantable energy source and to the energy consuming part, the energy provider being adapted to provide a burst of energy to the energy consuming part; and
subsequently powering the energy consuming part using the implantable energy source.

According to some embodiments, a maximum energy consumption of the energy consuming part is higher than the maximum energy capable of being delivered by the implantable energy source without causing damage to the implantable energy source, and
wherein the energy provider is adapted to deliver an energy burst corresponding to difference between the required energy consumption and the maximum energy capable of being delivered by the implantable energy source.

According to some embodiments, the method further comprises the step of:
charging the energy provider using the implantable energy source.

According to some embodiments, the initiating an energy consuming part comprises transitioning a control unit of the implant from a sleep mode to an operational or active mode.

According to some embodiments, the method further comprises
wirelessly charging the implantable energy source, the implantable energy source being connected to an internal charger, by controlling a receipt of electrical power from an external energy source at the implantable charger.

According to some embodiments, the method further comprises
wirelessly charging the implantable energy source, the implantable energy source being connected to an internal charger, by transmission of electrical power from an external energy source by the implantable charger.

### Aspect 309SE eHealth Broadcasting Data—Broadcasting Sensor Data from Implant—Embodiments of Aspect 309SE of the Disclosure

According to a first part of aspect 309SE an implant for transmitting sensor data is provided. The implant comprises:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant.

According to some embodiments, the communication unit is configured to broadcast the information using a short to mid-range transmitting protocol.

According to some embodiments, the information is broadcasted using at least one of: Radio Frequency type protocol, RFID type protocol, WLAN type protocol, Bluetooth type protocol, BLE type protocol, NFC type protocol, 3G/4G/5G type protocol. GSM type protocol, or Bluetooth 5.

According to some embodiments, the implant further comprises a control unit connected to the sensor and to the communication unit, wherein the control unit is configured to anonymize the information.

According to some embodiments, the implant further comprises a control unit connected to the sensor and to the communication unit, wherein the control unit is configured to encrypt the information.

According to some embodiments, the communication unit further is configured to broadcast the information periodically.

According to some embodiments, the implant further comprises a control unit configured to cause the communication unit to broadcast the information in response to a second parameter being above a predetermined threshold.

According to some embodiments, the sensed parameter is a pressure (such as a pressure at a sphincter or an organ of a patient, or a pressure at a hydraulic reservoir of the implant), a predetermined temperature interval or threshold (such as a temperature of the patient, or a temperature of a processing unit, a control unit, a power supply, or another part of the implant).

According to some embodiments, the implant further comprises an implantable energy source and an energy source indicator, wherein the energy source indicator is configured to indicate a functional status of the implantable energy source.

According to some embodiments, the functional status indicates at least one of charge level and temperature of the implantable energy source.

According to some embodiments, the functional parameter is a parameter relating to the internal control unit.

According to some embodiments, a system comprising the implant according to any preceding embodiment, and an external device comprising a receiver for receiving data from the implant and a transmitter for transmitting data is provided. The external device is configured to receive the broadcasted information, encrypt the received information using a key and transmit the encrypted received information.

According to some embodiments, the internal device is configured to transmit the data using the body of the patient as a conductor, and the external device is configured to receive the data via the body.

According to some embodiments, the communication unit of the implant is configured to transmit the data wirelessly to the external device.

According to a second part of aspect 309SE, a method for transmitting data from an implant comprising a processor and a communication unit is provided. The method comprising:
obtaining sensor measurement data via a sensor connected to or comprised in the implant, the sensor measurement relating to at least one physiological parameter of the patient or a functional parameter of the implant, and
transmitting, by a communication unit, the sensor measurement data in response to the sensor measurement being above a predetermined threshold,
wherein the sensor is configured to periodically sense the parameter.

According to some embodiments, the transmitting comprises broadcasting the sensor measurement data to an external device.

According to some embodiments, the broadcasting is performed using a short to mid-range transmitting protocol.

According to some embodiments, the transmitting comprises using at least one of a:
Radio Frequency type protocol
RFID type protocol
WLAN type protocol
Bluetooth type protocol
BLE type protocol
NFC type protocol
3G/4G/5G type protocol
GSM type protocol.

According to some embodiments, the method further comprises anonymizing, by the processor, the sensor measurement data before it is transmitted.

According to some embodiments, the method further comprises encrypting the sensor measurement data, using an encryptor comprised in the processing unit, before it is transmitted.

According to some embodiments, the obtaining and the transmitting is performed periodically.

According to some embodiments, the sensor measurement data is transmitted in response to a second parameter being above a predetermined threshold.

According to some embodiments, the parameter is a pressure, such as a pressure at a sphincter or an organ of a patient or a pressure at a hydraulic reservoir of the implant, a predetermined temperature interval or threshold, such as a temperature of the patient, or a temperature of a processing unit, a control unit, a power supply, or another part of the implant.

According to some embodiments, the implant comprises an implantable energy source and an energy source indicator, and wherein the energy source indicator is configured to indicate a functional status of the implantable energy source, and wherein the sensor measurement comprises data related to the energy source indicator.

According to some embodiments, the functional parameter is a parameter relating to the internal control unit, such as, for example, a free memory or free storage, available processing power, a temperature, or a battery indicator.

According to some embodiments, the method further comprises
receiving the sensor measurement data at an external device, and
at the external device, encrypting the sensor measurement data using a key to obtain encrypted data, and
transmitting the encrypted data.

According to some embodiments, the transmitting is performed wirelessly.

According to some embodiments, the internal communication unit comprises a conductive member, and the transmitting comprises transmitting, via the conductive member, the sensor measurement data using the body as a conductor.

According to some embodiments, the transmitting comprises transmitting the sensor measurement to an internal processor configured to cause a sensation generator to cause a sensation detectable by the patient in which the implant is implanted.

According to a third part, a computer program product comprising a computer-readable storage medium with instructions adapted to carry out the method of any one of the second part of aspect 309SE and/or with instructions adapted to carry out an action in any of the implant embodiments of the first part of aspect 309SE, when executed by a computing unit in an external device having processing capability is provided.

### Aspect 310SE eHealth Double Encryption—Double Encryption—Embodiments of Aspect 310SE of the Disclosure

According to a first part of aspect 310SE, a system for transmitting data between an implant and an external device is provided. The system comprises:
an implant comprising:
a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device.

According to some embodiments, the encryption unit is configured to encrypt the data to be transmitted using a second key.

According to some embodiments, the first key or the second key is implant specific information, a secret key associated with the external device, an identifier of the implant or an identifier of the communication unit.

According to some embodiments, the second key is a key transmitted by the external device to the internal device.

According to some embodiments, the second key is a combined key comprising a third key received by the implant form the external device.

According to some embodiments, the first key is a combined key comprising a fourth key, wherein the fourth key is received by the external device from a verification unit connected to or comprised in the external device.

According to some embodiments, the verification unit is configured to receive authentication input from a user, for authenticating the communication between the implant and the external device.

According to some embodiments, the authentication input is a code.

According to some embodiments, the authentication input is based on a biometric technique selected from the list of a fingerprint, a palm vein structure, image recognition, face recognition, iris recognition, a retinal scan, a hand geometry, and genome comparison.

According to some embodiments, the verification unit is configured to receive a fingerprint from a fingerprint reader.

According to some embodiments, the information is broadcasted using a short to mid-range transmitting protocol.

According to some embodiments, the information is transmitted using at least one of:
Radio Frequency type protocol
RFID type protocol
WLAN type protocol
Bluetooth type protocol
BLE type protocol
NFC type protocol
3G/4G/5G type protocol
GSM type protocol.
Bluetooth 5

According to some embodiments, the internal device comprises a first conductive member and the external device comprises a second conductive member, wherein the first and the second conductive members are configured to transmit the data using the body as a conductor.

According to some embodiments, the communication unit is configured to encrypt the data before transmitting the data.

According to some embodiments, the external device is configured to decrypt the received data and encrypt it before transmitting the data to the third device.

According to some embodiments, the external device is configured to transmit a request for data to the communication unit, and the communication unit is configured to in response to a request for data transmit the data to the external device.

According to some embodiments, the communication unit further is configured to broadcast the information periodically.

According to some embodiments, the system is further comprising an internal control unit configured to cause the communication unit to broadcast the information in response to a second parameter being above a predetermined threshold.

According to a second part of aspect 310SE, a method for encrypted communication between an implant, when implanted in a patient's body, and an external device is provided. The method comprising:
encrypting, by the implant, data relating to the implant or the operation thereof;
transmitting, by a first communication unit comprised in the implant, the data;
receiving, by a second communication unit comprised the external device, the data;
encrypting, by the external device, the data using an encryption key to obtain encrypted data; and
transmitting the encrypted data to a third external device.

According to some embodiments, the encrypting, by the implant, comprises encrypting the data using a second key.

According to some embodiments, the first or the second key is implant specific information, a secret key associated with the external device, an identifier of the implant or an identifier of the communication unit.

According to some embodiments, the second key is a key transmitted by the external device to the internal device.

According to some embodiments, the second key is a combined key comprising a third key; and the method further comprises: receiving, at the implant via a conductive member or wirelessly, the third key from the external device.

According to some embodiments, the method is further comprising:
receiving, at the external device, a fourth key from a verification unit connected to or comprised in the external device,
wherein the verification unit is configured to receive authentication input from a user, for authenticating the communication between the implant and the external device, and
wherein the first key is a combined key comprising a fourth key.

According to some embodiments, the authentication input is a code.

According to some embodiments, the authentication input is based on a biometric technique selected from the list of a fingerprint, a palm vein structure, image recognition, face recognition, iris recognition, a retinal scan, a hand geometry, and genome comparison.

According to some embodiments, the verification unit is configured to receive a fingerprint from a fingerprint reader.

According to a third part of aspect 310SE, a computer program product is provided. The computer program product comprising a computer-readable storage medium with instructions adapted to carry out the method of any one of embodiments of the second part of aspect 310SE and/or with instructions adapted to carry out an action in any of the embodiments of the first part of aspect 310SE, when executed by a computing unit in an external device having processing capability.

### Aspect 311SE eHealth Data Integrity—Verifying Data Integrity from/to Implant and from/to External Device—Embodiments of Aspect 311SE of the Disclosure

According to a first part of aspect 311SE, a method for evaluating a functional parameter of an implant implanted in a patient, the implant comprising a processor, a sensor for measuring the functional parameter, and an internal communication unit, is provided. The method comprising:
measuring, using the sensor, the functional parameter to obtain measurement data,
establishing a connection between the internal communication unit and an external device configured to receive data from the implant,
determining, by the processor, a cryptographic hash or a metadata relating to the measurement data and adapted to be used by the external device to verify the integrity of the received data, and
transmitting the cryptographic hash or metadata, and
transmitting, from the communication unit, the measurement data.

According to some embodiments, the method is further comprising, at the external device,
receiving the transmitted cryptographic hash or metadata,
receiving the measurement data, and
verifying the integrity of the measurement data with the cryptographic hash, metadata or information relating to the functional parameter.

According to some embodiments, the cryptographic hash or metadata comprises a cryptographic hash, and wherein the verifying the integrity of the measurement data comprises:
calculating a second cryptographic hash for the received measurement data using a same cryptographic hash algorithm as the processor, and
determining that the measurement data has been correctly received based on that the cryptographic hash and the second cryptographic hash are equal.

According to some embodiments, the cryptographic hash algorithm comprises one of MD5, SHA1, or SHA 256.

According to some embodiments, the cryptographic hash is a signature obtained by using a private key of the implant, and wherein the verifying, by the external device, comprises verifying the signature using a public key corresponding to the private key.

According to some embodiments, the cryptographic hash or metadata comprises a metadata, and wherein the verifying the integrity of the data comprises:
obtaining a second metadata for the received measurement data relating to the functional parameter, and
determining that the data has been correctly received based on that metadata and the second metadata are equal.

According to some embodiments, the metadata comprises: a length of the data, a timestamp, or a sensor measurement.

According to some embodiments, the method is further comprising, at the external device, evaluating the measurement data relating to the functional parameter.

According to some embodiments, the sensor is a pressure sensor, an electrical sensor, a clock, a temperature sensor, a motion sensor, an optical sensor, a sonic sensor, an ultrasonic sensor.

According to some embodiments, the functional parameter is at least one of a temperature, a pressure, a battery status indicator, a time period length, or a pressure at a sphincter.

According to some embodiments, the method is further comprising, at the external device, to determining, based on the evaluating, that the implant is functioning correctly.

According to some embodiments, the method is further comprising, at the external device, determining based on the evaluating that the implant is not functioning correctly.

According to some embodiments, the method is further comprising
sending, from the external device, a corrective command to the implant,
receiving the corrective command at the implant, and
correcting the functioning of the implant according to the corrective command.

According to some embodiments, the transmitting of the measurement data is transmitted in a plurality of data packets,
wherein the cryptographic mash or metadata comprises a plurality of cryptographic hashes or metadata each corresponding to a respective data packet, and
wherein the transmitting of each the cryptographic hashes or metadata is performed for each of the corresponding data packets.

According to some embodiments, the method is for evaluating a pressure at a sphincter of the patient.

According to a second part of aspect 311SE, a method of communicating instructions from an external device to an implant implanted in a patient is provided. The method comprising:
establishing a first connection between the external device and the implant,
establishing a second connection between a second external device and the implant,
transmitting, from the external device, a first set of instructions to the implant over the first connection,
transmitting, from the second external device, a first cryptographic hash or metadata corresponding to the first set of instructions to the implant,
at the implant, verifying the integrity of the first set of instructions and the first cryptographic hash, based on the first cryptographic hash.

According to some embodiments, the verifying of the integrity of the first set of instructions comprises a cyclic redundancy check.

According to some embodiments, the cryptographic hash or metadata comprises a cryptographic hash, and wherein the verifying the integrity of the first set of instructions comprises:
calculating a second cryptographic hash for the received first set of instructions using a same cryptographic hash algorithm as the processor, and
determining that the first set of instructions has been correctly received based on that the cryptographic hash and the second cryptographic hash are equal.

According to some embodiments, the cryptographic hash algorithm comprises one of: MD5, SHA1, or SHA 256.

According to some embodiments, the cryptographic hash is a signature obtained by using a private key of the implant, and wherein the verifying comprises verifying the signature using a public key corresponding to the private key.

According to some embodiments, the cryptographic hash or metadata comprises a metadata, and wherein the verifying the integrity of the data comprises:
obtaining a second metadata for the received first set of instructions, and
determining that the first set of instructions has been correctly received based on that metadata and the second metadata are equal.

According to some embodiments, the metadata comprises at least one of a length of the data, or a timestamp.

According to some embodiments, the external device is separate from the second external device.

According to some embodiments, communication using the second connection is performed using a different protocol than a protocol used for communication using the first communication channel.

According to some embodiments, the first connection is a wireless connection and the second connection is an electrical connection.

According to some embodiments, the second connection is an electrical connection using the patient's body as a conductor.

According to some embodiments, the method is further comprising:
transmitting, by the implant, information relating to the received first set of instructions,
receiving, by the external device, the information, and
verifying, by the external device, that the information corresponds to the first set of instructions sent by the external device.

According to some embodiments, the information comprises a length of the first set of instructions.

According to some embodiments, the method is further comprising:
at the implant, verifying the authenticity of the first set of instructions by
   i. calculating a second cryptographic hash for the first set of instructions,
   ii. comparing the second cryptographic hash with the first cryptographic hash,
   iii. determining that the first set of instructions are authentic based on that the second cryptographic hash is equal to the first cryptographic hash
upon verification of the authenticity of the first set of instructions, storing them at the implant.

According to some embodiments, the first set of instructions comprises a cryptographic hash corresponding to a previous set of instructions.

According to some embodiments, the method is further comprising:
measuring, by the implant using a first sensor, a parameter relating to the body of the patient to obtain a first measurement,
measuring, by the external device using a second sensor, the parameter relating to the body of the patient to obtain a second measurement,
wherein the first set of instructions comprises the second measurement relating to the body of the patient, and wherein the verification of the authenticity of the first set of instructions comprises comparing the first and the second measurements.

According to some embodiments, the first and second parameters relate to a pulse of the patient, a respiration rate of the patient, a temperature of the patient, a sound of the patient, or a physical movement of the patient.

According to some embodiments, the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.

According a second part of aspect 311SE, a system for communication instructions is provided, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash.

According to some embodiments, the internal controller is configured to verify the integrity of the first set of instructions using a cyclic redundancy check.

According to some embodiments, the cryptographic hash or metadata comprises a cryptographic hash, and wherein the internal controller is configured to verifying the integrity of the first set of instructions by:
calculating a second cryptographic hash for the received first set of instructions using a same cryptographic hash algorithm as the processor, and
determining that the first set of instructions has been correctly received based on that the cryptographic hash and the second cryptographic hash are equal.

According to some embodiments, the cryptographic hash algorithm comprises at least one of MD5, SHA1 or SHA256.

According to some embodiments, the cryptographic hash is a signature obtained by using a private key of the implant, and wherein the internal controller is configured to verifying the first set of instructions by the signature using a public key corresponding to the private key.

According to some embodiments, the cryptographic hash or metadata comprises a metadata, and wherein the internal controller is configured to verifying the integrity of the data by:
obtaining a second metadata for the received first set of instructions, and
determining that the first set of instructions has been correctly received based on that metadata and the second metadata are equal.

According to some embodiments, the metadata comprises at least one of: a length of the data, and/or a timestamp.

According to some embodiments, the external device is separate from the second external device.

According to some embodiments, the internal controller is configured to communicate with the second external device using a different protocol than a protocol used for communication with the external device.

According to some embodiments, the internal communication unit comprises a wireless transceiver for communication with the external device, and a conductive member for communicating with the second external device, wherein the second external device comprises a second conductive member.

According to some embodiments, the communication between the internal communication unit and the second external device is performed using the patient's body as a conductor.

According to some embodiments, the internal controller is configured to transmit information relating to the received first set of instructions to the external device, and the external device is configured to confirm that the information relates to the first set of instructions transmitted by the external device.

According to some embodiments, the internal controller is configured to:
calculating a second cryptographic hash for the first set of instructions,
comparing the second cryptographic hash with the first cryptographic hash,
determining that the first set of instructions are authentic based on that the second cryptographic hash is equal to the first cryptographic hash, and
upon verification of the authenticity of the first set of instructions, storing them at the implant.

According to some embodiments, the external device is configured to transmit the first set of instructions, and wherein the first set of instructions comprises a cryptographic hash corresponding to a previous set of instructions.

According to some embodiments, the internal controller is connected to or comprising a first sensor adapted to obtain a measurement of a parameter relating to the body of the patient,
the external device is connected to or comprising a second sensor adapted to obtain a measurement of the parameter relating to the body of the patient,
wherein the first set of instructions comprises the second measurement, and wherein the internal controller is configured to verify the authenticity of the first set of instructions at least based on a comparison of the first and second measurements.

According to some embodiments, the first and second parameters relate to a pulse of the patient, a respiration rate of the patient, a temperature of the patient, a sound of the patient, or a physical movement of the patient.

According to some embodiments, the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.

According to a third part of aspect 311 SE, a computer program product is provided. The computer program product comprises a computer-readable storage medium with instructions adapted to carry out the method of any one of the embodiments of the first part of aspect 307SE and/or with instructions adapted to carry out an action in any of the system embodiments of the second part of aspect 311SE, when executed by a computing unit in an external device having processing capability.

### Aspect 312SE eHealth Programming Predefined Steps—Programming Via Predefined Steps—Embodiments of Aspect 312SE of the Disclosure

According to a first part of aspect 312SE, a programmable or updatable implant is provided. The implant comprises:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps.

According to some embodiments, the predefined program steps comprise setting a variable related to a pressure, a time, a minimum or maximum temperature, a current, a voltage, an intensity, a frequency, an amplitude of electrical stimulation, a feedback mode (such as sensoric or other), a post-operative mode or a normal mode, a catheter mode, a fibrotic tissue mode (for example, semi-open), an time open after urination, a time open after urination before bed-time.

According to some embodiments, the verification function is configured to reject the update in response to the update comprising program steps not comprised in the set of predefined program steps.

According to some embodiments, the verification function is configured to allow the update in response to the update only comprising program steps comprised in the set of predefined program steps.

According to some embodiments, the internal communication unit is configured to communicate with the external device via a first wireless connection for receiving the update to the second control program, and a second connection for performing an authentication of the communication with the external device.

According to some embodiments, the second connection is a wireless short-range connection.

According to some embodiments, the authentication second connection is an electrical connection using the patient's body as a conductor.

According to some embodiments, the internal computing unit is further configured to, upon verification, installing the update.

According to some embodiments, the internal computing unit has a sleep mode and an active mode, and the implant further comprises a sensor configured to detect a wake signal, and wherein the implant is configured to in response to a detected wake signal set the internal computing unit to the active mode.

According to some embodiments, the sensor is configured to detect an acoustic signal as wake signal or wherein the sensor is configured to detect a magnetic signal as the wake signal.

According to some embodiments, the sensor is configured to detect the received signal strength of a signal, and the implant is further configured to set the internal computing unit to the active mode in response to the sensor detecting a signal exceeding a threshold signal strength.

According to some embodiments, the implant is further comprising a second internal computing unit, and wherein the implant is configured to set the internal computing unit to the active mode via the second internal computing unit.

According to some embodiments, the internal computing unit in the sleep mode is substantially without power, and wherein setting the internal computing unit in the active mode comprises providing the internal computing unit with power.

According to some embodiments, the implant comprises an energy controller for controlling the power supplied to the internal computing unit.

According to some embodiments, the sensor is configured to provide the energy controller with a second wake signal in response to detecting the wake signal, and wherein the energy controller is configured to set the computing unit in the active mode in response to the second wake signal.

According to some embodiments, the sensor is configured to detect the received signal strength of a signal, and the internal control unit is further configured to set the internal computing unit to the active mode in response to the sensor detecting a signal exceeding a threshold signal strength.

According to some embodiments, the wake signal comprises a predetermined signal pattern, and the implant is further configured to set the processing unit to the active mode in response to the sensor detecting the predetermined signal pattern.

According to some embodiments, the sensor is a hall effect sensor, a fluxgate sensor, an ultra-sensitive magnetic field sensor or a magneto-resistive sensor.

According to some embodiments, the sensor comprises a third coil having an iron core.

According to some embodiments, the sensor is comprised in the internal communication unit.

According to some embodiments, the implant is further comprising a frequency detector, communicatively coupled to the internal computing unit, and configured to detect a frequency for data communication between the internal communication unit and an external device configured to transmit a frequency indicator signal.

According to some embodiments, the frequency detector comprises an antenna.

According to some embodiments, the internal communication unit comprises a coil or a high-sensitivity magnetic field detector for communicating with the external device.

According to some embodiments, the implant is further comprising a sensation generator configured to generate a sensation detectable by a sense of the patient, the sensation generator being communicatively coupled to the internal control unit and being configured to, upon request, generate the sensation when the implant is implanted in the patient.

According to some embodiments, the sensation generator is configured to receive the request from the internal control unit of the implant.

According to some embodiments, the sensation generator is configured to create the sensation or sensation components by at least one of:
a vibration of the sensation generator;
producing a sound;
providing a photonic signal;
providing a light signal;
providing an electric signal; and
a heat signal.

According to some embodiments, the sensation generator is configured to be implanted in the patient.

According to some embodiments, the sensation generator is configured to be worn in contact with the skin of the patient.

According to some embodiments, the sensation generator is configured generate the sensation without being in physical contact with the patient.

According to a second part of aspect 312SE, a method for programming an implant by an external device is provided. The implant comprising an internal computing unit configured to control a function of said implant and an internal memory configured to store: a first control program for controlling the internal computing unit, a second, updatable or configurable, control program for controlling said function of said implant, and a set of predefined program steps for updating the second control program, the external device being configured to communicate with the implant via a first connection. The method comprising:
providing, at the internal computing unit, a set of predefined program steps for updating the second control program;
transmitting, by the external device, an update comprising a subset of the predefined program steps over the first connection;
receiving, at the internal computing unit, the update,
verifying, by the internal computing unit, that the update comprises a subset of the predefined program steps, and
upon verification of the instructions, running the update at the implant.

According to some embodiments, the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.

According to some embodiments, the verifying comprises rejecting the update in response to the update comprising program steps not comprised in the set of predefined program steps.

According to some embodiments, the verifying comprises allowing the update in response to the update only comprising program steps comprised in the set of predefined program steps.

According to some embodiments, the method is further comprising:
authenticating the communication between the implant and the external device over a second connection.

According to some embodiments, the second connection is a wireless short-range connection.

According to some embodiments, the second connection is an electrical connection using the patient's body as a conductor.

According to some embodiments, the method is further comprising, upon verification, installing the update.

According to some embodiments, the method is further comprising:
monitoring for signals by a sensor connected to the internal computing unit;
providing, from a signal provider comprised in the external control unit, a wake signal;
setting, by the internal computing unit and in response to a detected wake signal, a mode of a portion of the internal control unit from a sleep mode to an active mode.

According to some embodiments, the portion of the internal computing unit is the first control program or the second control program.

According to some embodiments, the method is further comprising
detecting, using a frequency detector, a frequency for the first communication channel between a first communication unit and a second communication unit, the first communication unit being associated with the internal control unit and the second communication unit being associated with the external device,
wherein the frequency detector is communicatively coupled to the internal computing unit.

According to some embodiments, the method is further comprising:
determining, using the frequency detector, the frequency for the first communication channel.

According to some embodiments, the method is further comprising:
generating, using a sensation generator communicatively coupled to the internal control unit, a sensation detectable by a sense of the patient in response to verifying the update, in response to running the update or in response to the update being installed at the implant.

According to some embodiments, the generating comprises at least one of:
providing a vibration of the sensation generator;
producing a sound;
providing a photonic signal;
providing a light signal;
providing an electric signal; and
providing a heat signal.

According to a third part of aspect 312SE, a computer program product is provided. The computer program product comprises a computer-readable storage medium with instructions adapted to carry out the method of any one of the embodiments of the second part of aspect 312SE and/or with instructions adapted to carry out an action in any of the implant embodiments of the first part of aspect 312SE, when executed by a computing unit in an external device having processing capability.

### Aspect 313SE eHealth Watchdog—Safety Reset Function—Embodiments of Aspect 313SE of the Disclosure

According to a first part of aspect 313SE, a programmable or updatable implant is provided. The implant comprises:
an internal processor comprising:
a first control program for controlling a function of the implant, and
   a first reset function, said first reset function being configured to restart or reset said first control program in response to:
a timer of the first reset function has not been reset, or
a malfunction in the first control program.

According to some embodiments, the first control program comprises a second reset function for resetting the timer of the first reset function.

According to some embodiments, the first reset function comprises a timer and the second reset function is configured to reset the timer.

According to some embodiments, the reset function comprises a first reset function and a second reset function, wherein the first reset function is configured to trigger a corrective function for correcting the first control program, and wherein the second reset function is configured to restart the first control program after the corrective function has been triggered.

According to some embodiments, the first or second reset function is configured to invoke a hardware reset by activating an internal or external pulse generator which is configured to create a reset pulse for the internal computing unit or the first control program.

According to some embodiments, the internal computing unit is configured to have an active mode and a sleep mode, and wherein the first reset function is configured to have an active mode and a sleep mode corresponding to the active mode and the sleep mode of the internal computing unit.

According to some embodiments, the implant is further comprising a sensor for measuring a physiological parameter of the patient or a parameter of the implant, and wherein the sensor is configured to invoke the reset function in response to the parameter being above or below a predetermined value.

According to some embodiments, the sensor is a pressure sensor adapted to measure a pressure in a part of the implant.

According to some embodiments, the pressure sensor is configured to measure a pressure in a reservoir or a restriction device of the implant.

According to some embodiments, the sensor is a pressure sensor adapted to measure a pressure in an organ of the patient's body.

According to some embodiments, the reset function is configured to be invoked by an electrical reset pulse, and wherein the sensor is adapted to invoke the reset function by activating an internal or external pulse generator which is configured to create a reset pulse for the reset function.

According to some embodiments, the physiological parameter of the patient or a parameter of the implant is a temperature.

According to some embodiments, the reset function comprises invoking a second control program comprising a safety measure.

According to some embodiments, the safety measure comprises controlling a function of the implant.

According to some embodiments, the internal computing unit is configured to invoke the reset function periodically.

According to some embodiments, periodically comprises every 24 hours.

According to some embodiments, the internal computing unit further comprises a monitoring function for monitoring a function of the implant or the first control program, and wherein the reset function is configured to in response to an incorrect or absent response for the monitoring program, reset or restart the first control program.

According to some embodiments, the internal computing unit has an active mode and a sleep mode, the sleep mode having a lower energy consumption than the active mode, and wherein the implant further comprises an internal control unit connected to the internal computing unit and adapted to control the mode of the internal computing unit.

According to some embodiments, the implant further comprises a second sensor for measuring a physiological parameter of the patient or a parameter of the implant, the second sensor being connected to the internal control unit, and, in response to a sensor measurement differing from, exceeding or being less than a predetermined value, setting the internal computing unit in the active mode.

According to some embodiments, the sensor is configured to measure the physical parameter periodically.

According to some embodiments, the sensor and the second sensor is the same sensor.

According to some embodiments, the sensor is a pressure sensor.

According to some embodiments, the sensor is adapted to measure a pressure in one or more of: an organ of a patient; a reservoir; and a restriction device.

According to some embodiments, the implant further comprises a third sensor for detecting a wake signal from an external device, the second sensor being connected to the internal control unit, and, in response to a measurement differing from, exceeding or being less than a predetermined value, setting the internal computing unit in the active mode.

According to some embodiments, the signal is a magnetic signal or an acoustic signal.

According to some embodiments, the sensor is configured to detect the received signal strength of a signal; and the internal control unit is further configured to set the processing unit to the active mode in response to the sensor detecting a signal exceeding a threshold signal strength.

According to some embodiments, the wake signal comprises a predetermined signal pattern; and the internal control unit is further configured to set the processing unit to the active mode in response to the sensor detecting the predetermined signal pattern.

According to some embodiments, the sensor comprises a hall effect sensor, a fluxgate sensor, an ultra-sensitive magnetic field sensor, a magneto-resistive sensor, a coil, or a coil having an iron core.

According to some embodiments, the internal control unit comprises a first communication unit for receiving and/or transmitting data from and/or to the external control unit; and the external control unit comprises a second communication unit for transmitting and/or receiving data to and/or from the internal control unit.

According to some embodiments, the implant is further comprising a frequency detector, communicatively coupled to the internal control unit, and configured to detect a frequency for data communication between the first communication unit and the second communication unit.

According to some embodiments, the frequency detector comprises an antenna.

According to a second part of the twenty-eighth aspect, a method for controlling a control program of an implant, when implanted in a patient, is provided. The implant comprising a processor for running the first control program, and the method comprising: executing the first control program at the internal computing unit; executing a first reset function; resetting or restarting the first control program by the first reset function in response a detection of a malfunction in the first control program.

According to some embodiments, the resetting or restarting of the first control program comprises triggering a corrective function for correcting the first control program.

According to some embodiments, the method is further comprising:
periodically resetting, by the first control program, the first reset function,
wherein the detecting of a malfunction comprises determining that the first reset function has not been reset for a predetermined period of time.

According to some embodiments, the detecting of a malfunction comprises detecting that a sensor measurement relating to a physiological parameter of the patient or a parameter of the implant being less than, exceeding or differing from a predetermined value.

According to some embodiments, the sensor measurement relates to a pressure in a part of the implant.

According to some embodiments, the sensor measurement is related to a pressure in a reservoir or a restriction device of the implant.

According to some embodiments, the sensor measurement is related to a pressure in an organ of the patient's body.

According to some embodiments, the physiological parameter of the patient or a parameter of the implant is a temperature.

According to some embodiments, the reset function comprises invoking a second control program comprising a safety measure.

According to some embodiments, the safety measure comprises controlling a function of the implant.

According to some embodiments, periodically comprises every 24 hours.

According to some embodiments, the method is further comprising:
monitoring a function of the implant or the first control program,
and wherein the reset function is configured to in response to an incorrect or absent response for the monitoring program, reset or restart the first control program.

According to a third part of the twenty-eighth aspect, a computer program product comprising a computer-readable storage medium with instructions adapted to carry out the method of any one of embodiments of the second part of the twenty-eighth aspect, and/or with instructions adapted to carry out an action in any of the implant embodiments of the first part of the twenty-eighth aspect, when executed by a computing unit in an external device having processing capability, is provided.

### Aspect 314SE eHealth Logging—Update Confirmation—Embodiments of Aspect 314SE of the Disclosure

According to a first part of aspect 314SE, a method for updating a control program of an internal computing unit comprised in an implant is provided. The implant is adapted for communication with a first external device and a second external device, and the method comprises:
receiving, by the internal computing unit, an update or configuration to the control program from the first external device, wherein the update is received using a first communication channel;
installing, by the internal computing unit, the update; and
transmitting, by the internal computing unit, logging data relating to the receipt of the update or configuration and/or logging data relating to an installation of the update to the second external device using the second communication channel;
wherein the first and the second communication channels are different communication channels.

According to some embodiments, the update or configuration comprises a set of instructions for the control program.

According to some embodiments, the steps comprises a subset of a set of predefined steps.

According to some embodiments, the method is further comprising
confirming, by a user or by an external control unit, that the update or configuration is correct based on the received logging data.

According to some embodiments, the logging data is related to the receipt of the update or configuration, and the internal computing unit is configured to install the update or configuration in response to receipt of a confirmation that the logging data relates to a correct set of instructions.

According to some embodiments, the method is further comprising:
installing, in response to the confirmation that the update or configuration is correct, the update or configuration.

According to some embodiments, the logging data is related to the installation of the update or configuration.

According to some embodiments, the method is further comprising:
activating the installation in response to the confirmation that the update or configuration is correct.

According to some embodiments, the update or configuration comprises a plurality of steps, and the receiving of the update or configuration further comprises receiving the plurality of steps in two or more subsets.

According to some embodiments, the method is further comprising confirming, by a user or by an external device, that each of the subsets are correct.

According to some embodiments, the method is further comprising
confirming that the installation is complete by producing a sound or a vibration detectable by the user.

According to some embodiments, the configuration or update comprises a value for a predetermined parameter.

According to some embodiments, the method is further comprising receiving, by the first external device, an update or a configuration to the control program by a user.

According to some embodiments, the method is further comprising:
selecting, by a user of the first external device, a step from a set of predetermined steps, to be comprised in the update or configuration, and/or
setting, by a user of the first external device, a value for a parameter to be comprised in the update or configuration.

According to some embodiments, the communication over the first communication channel is performed using a first network protocol, and communication over the second communication channel is performed using a second network protocol, the first and second protocols being different.

According to some embodiments, the network protocol is at least one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments, the second network protocol is at least one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments, the method is, after transmitting the logging data to the second external device, further comprising the step of:
verifying the update via a confirmation from the second external device via the second communication channel.

According to a second part of aspect 314SE, an implant adapted for communication with a first external device and a second external device, when the implant is adapted to be implanted in a patient, is provided. The implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels.

According to some embodiments, the update or configuration comprises a set of instructions for the control program.

According to some embodiments, the steps comprises a subset of a set of predefined steps.

According to some embodiments, the second external device is configured to confirm that the update or configuration is correct based on the received logging data.

According to some embodiments, the logging data is related to the receipt of the update or configuration, and the internal computing unit is configured to install the update or configuration in response to receipt of a confirmation that the logging data relates to a correct set of instructions.

According to some embodiments, the logging data is related to the installation of the update or configuration, and wherein the internal computing unit is configured to activate the installation in response to a confirmation that the update or configuration is correct.

According to some embodiments, the update or configuration comprises a plurality of steps, and the update or configuration is received by the internal computing unit in two or more sub steps.

According to some embodiments, the method is further comprising a sensation generator adapted to create a sensation detectable by the user.

According to some embodiments, the internal computing unit is configured to cause the sensation generator to create a sensation detectable by the user in response to the update or configuration being received, in response to the update or configuration being installer or in response to the update or configuration being confirmed.

According to some embodiments, the sensation generator is a vibrator or a speaker.

According to some embodiments, the configuration or update comprises a value for a predetermined parameter.

According to some embodiments, the configuration or update comprises a step from a set of predetermined steps.

According to some embodiments, the communication over the first communication channel is performed using a first network protocol, and communication over the second communication channel is performed using a second network protocol, the first and second protocols being different.

According to some embodiments, the network protocol is at least one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments, the second network protocol is at least one from the list of:
Radio Frequency type protocol
RFID type protocol
WLAN type protocol
Bluetooth type protocol
BLE type protocol
NFC type protocol
3G/4G/5G type protocol
GSM type protocol.

According to some embodiments, the second communication channel is an electrical connection.

According to a third part of aspect 314SE, a computer program product comprising a computer-readable storage medium with instructions adapted to carry out the method of any one of embodiments of the first part of aspect 314SE and/or with instructions adapted to carry out an action in any of the implant embodiments of the second part of aspect 314SE, when executed by a computing unit in an external device having processing capability, is provided.

### Aspect 315SE eHealth Sleeping Internal Control Unit—Sleep Mode for Internal Controller—Embodiments of Aspect 315SE of the Disclosure

According to first part of aspect 315SE, an implant having a controller with a sleep mode is provided. The implant comprises:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode.

According to some embodiments, the sensor is configured to measure periodically.

According to some embodiments, the sensor is a mechanical sensor.

According to some embodiments, the sensor comprises a pressure sensor, a piezoelectric sensor, or a bimetal.

According to some embodiments, the sensor is configured to measure a physiological parameter of the patient; and the sensor is a pressure sensor.

According to some embodiments, the pressure sensor is adapted to measure a pressure in one or more of: an organ of a patient; a reservoir; and a restriction device.

According to some embodiments, the sensor is configured to measure a parameter of the implant; and the sensor is adapted to measure one or more of: a battery status of a battery of the implant and a temperature of the implant.

According to some embodiments, the sensor is an analog sensor or a digital sensor.

According to some embodiments, the implant is further comprising a sensation generator configured to, upon request, generate a sensation detectable by a sense of the patient.

According to some embodiments, the sensation generator is configured to receive the request from the controller of the implant.

According to some embodiments, the request is generated by the controller in response to the sensor measurement having a value outside of the predetermined interval.

According to some embodiments, the sensation generator is configured to receive the request from an external controller.

According to some embodiments, the generated sensation comprises a plurality of sensation components.

According to some embodiments, the sensation generator is configured to create the sensation or sensation components by at least one of: a vibration of the sensation generator; producing a sound; providing a photonic signal; providing a light signal; providing an electric signal; and providing a heat signal.

According to some embodiments, the implant is further comprising an active unit, communicatively coupled to the processor, for performing controlling or monitoring a bodily function in the patient.

According to some embodiments, the sensor is configured to measure a physiological parameter of the patient; and the active unit is configured to perform the controlling or monitoring in response to a sensor measurement having a value outside of the predetermined interval, after the processor has been set in the active state. By “a value outside of a predetermined interval” it may be meant that the value is outside of an interval determined by a control unit, that the value is less than (or less than or equal) to a predetermined threshold, and/or that the value is exceeding (or exceeding or equal to) a predetermined threshold.

According to some embodiments, the controller further comprises a communication unit communicatively coupled to the processor, and the processor is configured to transmit data relating to the measurement via the communication unit.

According to some embodiments, the implant is further comprising a frequency detector, communicatively coupled to the controller and configured to detect a frequency for data communication to or from the communication unit.

According to some embodiments, the frequency detector comprises an antenna.

According to a second part of aspect 315SE, a system is provided. The system comprising:
the implant according to any of the embodiments of the first part of aspect 315SE; and
an external controller, adapted to be arranged outside of the patient's body, configured to communicate with the communication unit.

According to some embodiments, the external controller is a wireless remote control.

According to some embodiments, the communication unit is further configured to:
receive one or more control signals from the external controller, and
control an operation of the implant based on the one or more control signals, when the processor is in the active state.

According to some embodiments, the one or more control signals is selected from the group consisting of:
a sound signal;
an ultrasound signal;
an electromagnetic signal;
an infrared signal;
a visible light signal;
an ultraviolet light signal;
a laser signal;
a microwave signal;
a radio wave signal;
an X-ray radiation signal; and
a gamma radiation signal.

According to some embodiments, the system is further comprising a frequency detector, communicatively coupled to the external controller, and configured to detect a frequency for data communication between the communication unit and the external controller.

According to some embodiments, the frequency detector comprises an antenna.

According to some embodiments, the system is further comprising an external sensation generator adapted to be arranged outside of the patient's body and to, upon request, generate a sensation detectable by a sense of the patient.

According to some embodiments, the external controller is configured to generate the request.

According to some embodiments, the external sensation generator is configured to be worn in contact with the skin of the patient.

According to some embodiments, the external sensation generator is configured to generate the sensation without being in physical contact with the patient.

According to a third part of aspect 315SE, a method for controlling an implant implanted in a patient is provided. The method comprising:
measuring, with a passive sensor of a controller connected to or comprised in the implant, a physiological parameter of the patient or a parameter of the implant; and
in response to a sensor measurement having an value outside of a predetermined interval, setting, by the controller, a processor of the controller from a sleep mode to an active mode. By “a value outside of a predetermined interval” it may be meant that the value is outside of an interval, that the value is less than (or less than or equal) to a predetermined threshold, and/or that the value is exceeding (or exceeding or equal to) a predetermined threshold.

According to some embodiments, the measuring is carried out periodically.

According to some embodiments, the method is further comprising generating, with a sensation generator comprised in or connected to the implant, a sensation detectable by a sense of the patient.

According to some embodiments, the method is further comprising generating, by the controller, a request to generate a sensation with the sensation generator in response to the sensor measurement having a value outside of a predetermined interval.

According to some embodiments, the method is further comprising: performing, with an active unit comprised in or connected to the implant, a medical intervention in the patient.

According to some embodiments, the method is further comprising:
performing the medical intervention in response to a sensor measurement having a value outside of a predetermined interval, after setting the processor in the active state.

According to some embodiments, the method is further comprising:
detecting, using a frequency detector, a frequency for data communication to or from a communication unit, the frequency detector being communicatively coupled to the controller.

According to some embodiments, the detecting is initiated in response to setting the processor in the active state.

According to some embodiments, the method is further comprising:
exchanging data communications between the communication unit and an external controller, adapted to be arranged outside of the patient's body,
wherein the data communications comprise at least one of:
data relating to the measurement, and
one or more control signals transmitted by the external controller.

According to some embodiments, the method is further comprising:
controlling an operation of the implant based on the one or more control signals, when the processor is in the active state.

According to some embodiments, the method is further comprising:
detecting, using an external frequency detector, a frequency for data communication between the communication unit and the external controller, the external frequency detector being communicatively coupled to the external controller.

According to a fourth part of aspect 315SE, a computer program product comprising a computer-readable storage medium with instructions adapted to carry out the method of any one of embodiments of the third part of aspect 315SE, and/or with instructions adapted to carry out an action in any of the implant embodiments of the first part or any of the system embodiments of the second part, when executed by a computing unit in an external device having processing capability, is provided.

### Aspect 316SE eHealth Relay Instructions—Relaying of Instructions—Embodiments of Aspect 316SE

According to a first part of aspect 316SE, a method for transmitting an instruction from a first external device to an implant is provided. The method comprising:
transmitting an instruction for the implant from the first external device to a second external device, the instruction relating to a function of the implant,
encrypting, at the second external device and using a first encryption key, the instruction into an encrypted instruction, and
transmitting the encrypted instruction from the second external device to the implant,
decrypting, at the implant, the instructions using a second encryption key corresponding to the first encryption key.

According to some embodiments, the transmitting of the encrypted instruction from the second external device to the implant comprises:
transmitting the encrypted instruction from the second external device to the first external device, and
transmitting the encrypted instruction from the first external device to the implant.

According to some embodiments, the transmitting of the encrypted instruction from the second external device to the implant comprises:
transmitting the encrypted instruction from the second external device to a third external device, and
transmitting the encrypted instruction from the third external device to the implant.

According to some embodiments, the second external device is an encryption device communicatively coupled to the first external device, and wherein the communication of the instruction between the second external device and the implant is relayed through the first external device.

According to some embodiments, the method is further comprising, at the implant, running the instruction.

According to some embodiments, the method is further comprising receiving, at the first external device, the instruction.

According to some embodiments, the method is further comprising displaying, at the external device, a user interface for receiving the instruction.

According to some embodiments, the implant comprises a set of a predefined program steps, and wherein the method further comprises verifying, by the implant, that the received instruction is comprised in the predefined program steps.

According to some embodiments, the verifying comprises rejecting the instruction in response to the instruction not being comprised in the set of predefined program steps.

According to some embodiments, the verifying comprises allowing the instruction in response to the instruction being comprised in the set of predefined program steps.

According to some embodiments, the first external device and the implant are configured to communicate over a wireless connection.

According to some embodiments, the wireless connection comprises using at least one of the following protocols:
Radio Frequency type protocol
RFID type protocol
WLAN type protocol
Bluetooth type protocol
BLE type protocol
NFC type protocol
3G/4G/5G type protocol
GSM type protocol
Bluetooth 5.

According to some embodiments, the transmitting of data between the first external device and the second external device is performed a wireless connection.

According to some embodiments, the method is further comprising authenticating the connection between the first external device and the implant over which the encrypted instruction is to be transmitted.

According to some embodiments, the implant comprises an internal control unit for controlling a function of the implant, and wherein the internal control unit is configured to run the instruction.

According to a second part of aspect 316SE, A system for transmitting an instruction from a first external device to an implant is provided. The system comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant,
wherein the implant is configured to received and decrypt the instruction.

According to some embodiments, the second external device is configured to transmit the encrypted instruction by transmitting the encrypted instruction to the first external device, and wherein the first external device is configured to transmit the encrypted instruction to the implant.

According to some embodiments, the second external device is configured to transmit the encrypted instruction by transmitting the encrypted instruction to a third external device, and wherein the third external device is configured to transmit the encrypted instruction to the implant

According to some embodiments, the second external device is an encryption device communicatively coupled to the first external device, and wherein any communication between the implant and the second external device is relayed through the first external device.

According to some embodiments, the internal control unit is configured to run the decrypted instruction for controlling a function of the implant.

According to some embodiments, the first external device is configured to display a user interface for receiving the instruction.

According to some embodiments, the implant comprises a set of a predefined program steps, and wherein the implant is configured to verify that the received instruction is comprised in the predefined program steps.

According to some embodiments, the implant is configured to reject the instruction in response to the instruction not being comprised in the set of predefined program steps.

According to some embodiments, the implant is configured to allow the instruction in response to the instruction being comprised in the set of predefined program steps.

According to some embodiments, the first external device and the implant are configured to communicate over a wireless connection.

According to a third part of aspect 316SE, a computer program product comprising a computer-readable storage medium with instructions adapted to carry out the method of any one of embodiments of the first part and/or with instructions adapted to carry out an action in any of the system embodiments of the second part, when executed by a computing unit in an external device having processing capability, is provided.

### Aspect 317SE Energy General Microphone—Microphone Sensor—Embodiments of Aspect 317SE of the Disclosure

According to a first part of aspect 317SE, a controller for controlling an energized implant is provided. According to a first part of this aspect there is provided an implantable controller for controlling an energized implant, when implanted in a patient, the controller comprises a computing unit and at least one microphone, wherein the at least one microphone is configured to register a sound related to at least one of a bodily function, and a function of the implant.

According to some embodiments of the first part of aspect 317SE, the implantable controller further comprises at least one implantable housing for sealing against fluid, and wherein the computing unit and the microphone are placed inside of the housing.

According to some embodiments of the first part of aspect 317SE, wherein the computing unit is configured to derive a pulse of the patient from the registered sound related to a bodily function.

According to some embodiments of the first part of aspect 317SE, the computing unit is configured to derive information related to the patient urinating from the registered sound related to a bodily function.

According to some embodiments of the first part of aspect 317SE, the computing unit is configured to derive information related to a bowel activity of the patient from the registered sound related to a bodily function.

According to some embodiments of the first part of aspect 317SE, the computing unit is configured to derive information related to a functional status of the implant from the registered sound related to a function of the implant.

According to some embodiments of the first part of aspect 317SE, the computing unit is configured to derive information related to the functional status of an operation device of the implant, from the registered sound related to a function of the implant.

According to some embodiments of the first part of aspect 317SE, the computing unit is configured to derive information related to the functional status of at least one of: a motor, a pump and a transmission of the operation device of the implant from, the registered sound related to a function of the implant.

According to some embodiments of the first part of aspect 317SE, the implantable controller further comprises a transceiver, and wherein the controller is configured to transmit a parameter derived from the sound registered by the at least one microphone using the transceiver.

According to a second part of aspect 317SE there is provided a method of authenticating an energized implant implanted in a patent, performed in a system comprising the energized implant and an external device, the energized implant comprising at least one microphone, and a transmitter, and the external device comprising a receiver and a computing unit. The method comprises: registering a sound related to at least one of a bodily function and a function of the implant, using the at least one microphone, transmitting a signal derived from the registered sound, using the transmitter, receiving, in the external device, the signal derived from the registered sound, using the receiver, and comparing, in the external device, a parameter derived from the received signal with a reference parameter, using the computing unit.

According to some embodiments of the second part of aspect 317SE, the method further comprises the step of authenticating the energized implant on the basis of the comparison.

According to some embodiments of the second part of aspect 317SE, the method further comprises receiving, at the receiver of the external device, a parameter to be used as reference parameter.

According to some embodiments of the second part of aspect 317SE the step of receiving a parameter to be used as reference parameter comprises receiving the parameter from a sensor external to the patient.

According to some embodiments of the second part of aspect 317SE the registered sound is related to a pulse of the patient, and wherein the reference parameter is related to the pulse of the patient.

According to a third part of aspect 317SE there is provided a method of authenticating an energized implant implanted in a patent, performed in a system comprising the energized implant and an external device, the energized implant comprising at least one microphone, a receiver, and a computing unit, and the external device comprising a transmitter. The method comprising the steps of registering a sound related to at least one of a bodily function and a function of the implant, using the at least one microphone,
deriving a parameter from the sound using the computing unit, receiving, in the energized implant, a reference parameter, from the external device, using the receiver, and comparing, in the energized implant, the parameter derived from the sound with the received reference parameter, using the computing unit.

According to some embodiments of the third part of aspect 317SE, the method further comprises the step of authenticating the energized implant on the basis of the comparison.

According to some embodiments of the third part of aspect 317SE, the method further comprises receiving, at a receiver of the external device, a parameter to be used as reference parameter.

According to some embodiments of the third part of aspect 317SE, the step of receiving a parameter to be used as reference parameter comprises receiving the parameter from a sensor external to the patient.

According to some embodiments of the third part of aspect 317SE, the registered sound is related to a pulse of the patient, and wherein the reference parameter is related to the pulse of the patient.

### Aspect 318SE Energy Appetite Control Microphone—Microphone Sensor for Appetite Control—Embodiments of Aspect 318SE of the Disclosure

According to a first part of aspect 318SE there is provided an implantable controller for controlling an energized implant for stretching the stomach wall of a patient to thereby create satiety, when implanted in a patient. The controller comprises at least one microphone configured to register a sound related to the patient swallowing, and a computing unit configured to derive a parameter related to the patient swallowing from the sound.

According to some embodiments of the first part of aspect 318SE, the computing unit is configured to derive a parameter related to the size and/or shape and/or viscosity of a swallowed contents.

According to some embodiments of the first part of aspect 318SE, the computing unit is configured to determine if a swallowed content is a liquid or a solid.

According to some embodiments of the first part of aspect 318SE, the computing unit is configured to determine an accumulated amount of swallowed content over a time period.

According to some embodiments of the first part of aspect 318SE, the implantable controller further comprises a transmitter, and wherein the controller is configured to transmit the parameter derived from the sound registered by the at least one microphone using the transmitter.

According to some embodiments of the first part of aspect 318SE, the implantable controller further comprises a receiver wherein the controller is configured to receive a signal from an external device.

According to some embodiments of the first part of aspect 318SE, the computing unit is further configured to generate a control signal for controlling the energized implant for stretching the stomach wall of a patient on the basis of the derived parameter related to the patient swallowing, or the signal received from the external device, or a combination of the derived parameter related to the patient swallowing and the signal received from the external device.

According to a second part of aspect 318SE there is provided a system for controlling an energized implant for stretching the stomach wall of a patient to thereby create satiety, the system comprising an implantable controller for controlling the energized implant and an external device. The implantable controller comprising at least one microphone configured to register a sound related to the patient swallowing, a computing unit configured to derive a parameter related to the patient swallowing from the registered sound, a transmitter configured to transmit the derived parameter, and a receiver configured to receive control signals from the external device. Wherein the external device comprises a receiver configured to receive a parameter derived from a sound related to the patient swallowing, a computing unit configured to generate a control signal on the basis of the received parameter, and a transmitter configured to transmit the control signal to the implantable controller for controlling the energized implant for stretching the stomach wall of a patient to thereby create satiety.

According to some embodiments of the second part of aspect 318SE, the computing unit of the external device is configured to derive a parameter related to the size and/or shape and/or viscosity of a swallowed contents on the basis of the received parameter derived from the sound related to the patient swallowing.

According to some embodiments of the second part of aspect 318SE, wherein the computing unit of the external device is configured to determine if a swallowed content is a liquid or a solid on the basis of the received parameter derived from the sound related to the patient swallowing.

According to some embodiments of the second part of aspect 318SE, the computing unit of the external device is configured to determine an accumulated amount of swallowed content over a time period.

According to some embodiments of the second part of aspect 318SE, the computing unit of the external device is configured to generate the control signal on the basis of the accumulated amount of swallowed content over a time period.

According to a third part of aspect 318SE there is provided a method in an implantable controller for controlling an energized implant for stretching the stomach wall of a patient to thereby create satiety, when implanted in a patient, the implantable controller comprises at least one microphone and a computing unit. The method comprises the steps of registering a sound related to the patient swallowing, using the at least one microphone, and deriving a parameter related to the patient swallowing from the sound, using the computing unit.

According to some embodiments of the third part of aspect 318SE, the method further comprises deriving a parameter related to the size and/or shape and/or viscosity of a swallowed contents, using the computing unit.

According to some embodiments of the third part of aspect 318SE, the method comprises determining if a swallowed content is a liquid or a solid, using the computing unit.

According to some embodiments of the third part of aspect 318SE, the method further comprises determining an accumulated amount of swallowed content over a time period, using the computing unit.

According to some embodiments of the third part of aspect 318SE, wherein the implantable controller further comprises a transmitter, and wherein the method further comprises transmitting a parameter derived from the sound registered by the at least one microphone, to an external device, using the transmitter.

According to some embodiments of the third part of aspect 318SE, the implantable controller further comprises a receiver, and wherein the method further comprises receiving a signal from an external device.

According to some embodiments of the third part of aspect 318SE, the method further comprises generating a control signal for controlling the energized implant for stretching the stomach wall of a patient, using the computing unit, on the basis of: the derived parameter related to the patient swallowing, or the signal received from the external device, or a combination of the derived parameter related to the patient swallowing and the signal received from the external device.

According to a fourth part of aspect 318SE there is provided a method of authenticating an implantable controller for controlling an energized implant for stretching the stomach wall of a patient to thereby create satiety, performed in a system comprising the energized implant and an external device, the energized implant comprising at least one microphone, and a transmitter, and the external device comprising a receiver and a computing unit. The method comprising the steps of registering a sound related to the patient swallowing, using the at least one microphone, and transmitting a signal derived from the registered sound, using the transmitter, receiving, in the external device, the signal derived from the registered sound, using the receiver, and comparing, in the external device, a parameter derived from the received signal with a reference parameter, using the computing unit.

According to some embodiments of the fourth part of aspect 318SE, the method further comprises the step of authenticating the energized implant on the basis of the comparison.

According to some embodiments of the fourth part of aspect 318SE, the method further comprises receiving, at the receiver of the external device, a parameter to be used as reference parameter.

According to some embodiments of the fourth part of aspect 318SE, the step of receiving a parameter to be used as reference parameter comprises receiving the parameter from a sensor external to the patient.

According to some embodiments of the fourth part of aspect 318SE, the step of receiving the parameter from a sensor external to the patient comprises receiving the parameter from a sensor configured to sense the patient swallowing.

According to some embodiments of the fourth part of aspect 318SE, the step of receiving a parameter to be used as reference parameter comprises receiving input from the patient.

An external device configured for communication with an implantable medical device, when implanted in a patient, is provided. The external device comprises at least one first wireless transceiver configured for communication with the implantable medical device using a first network protocol, for determining a distance between the external device and the implantable medical device, and at least one second wireless transceiver configured for communication with the implantable medical device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of powering an energy consuming component of the implantable medical device and charging an implantable energy storage unit.

According to one embodiment, the second network protocol is a standard network protocol. The standard network protocol may be one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the second wireless transceiver comprises a Bluetooth transceiver.

According to one embodiment, the external device is further configured to communicate with a second external device using said at least one wireless transceiver.

According to one embodiment, the external device is configured for determining a distance between the external device and the implantable medical device by determining the RSSI.

According to one embodiment, a communication range of the first network protocol is less than a communication range of the second network protocol.

According to one embodiment, a frequency band of the first network protocol differs from a frequency band of the second network protocol.

According to one embodiment, the external device is configured to authenticate the implantable medical device if the determined distance between the external device and the implantable medical device is less than a predetermined threshold value.

According to one embodiment, the external device is configured to allow the transfer of data between the external device and the implantable medical device after the implantable medical device has been authenticated.

According to one embodiment, the external device is one from the list of: a wearable external device, and a handset.

An implantable medical device configured for communication with an external device is provided. The implantable medical device comprises at least one first wireless transceiver configured for communication with the external device using a first network protocol, for determining a distance between the external device and the implantable medical device, and at least one second wireless transceiver configured for communication with the external device using a second network protocol, for transferring data between the external device and the implantable medical device.

According to one embodiment, the first wireless transceiver comprises an UWB transceiver.

According to one embodiment, the first wireless transceiver is configured for transcutaneous energy transfer for at least one of: powering an energy consuming component of the implantable medical device, and charging an implantable energy storage unit.

According to one embodiment, the second network protocol is a standard network protocol, such as selected from the list of Radio Frequency type protocol, RFID type protocol, WLAN type protocol, Bluetooth type protocol, BLE type protocol, NFC type protocol, 3G/4G/5G type protocol, and GSM type protocol.

According to one embodiment, the second wireless transceiver comprises a Bluetooth transceiver.

According to one embodiment, the implantable medical device is further configured to communicate with a second external device using said at least one wireless transceiver.

According to one embodiment, the implantable medical device is configured for determining a distance between the external device and the implantable medical device by determining the RSSI.

According to one embodiment, a communication range of the first network protocol is less than a communication range of the second network protocol.

According to one embodiment, a frequency band of the first network protocol differs from a frequency band of the second network protocol.

According to one embodiment, the implantable medical device is configured to authenticate the external device if the determined distance between the external device and the implantable medical device is less than a predetermined threshold value.

According to one embodiment, the implantable medical device is configured to allow the transfer of data between the implantable medical device and the external device after the external device has been authenticated.

According to one embodiment, the implantable medical device comprises at least one of: an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries,
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
   an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
   an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A patient external device configured for communication with an implantable medical device, when implanted in a patient, is provided. The patient external device comprises a wireless communication unit configured for wireless transmission of control commands to the implantable medical device and configured for wireless communication with a patient display device, and a computing unit configured for running a control software for creating the control commands for the operation of the implantable medical device. The computing unit is configured to transmit a control interface as a remote display portal to a patient display device configured to display the control interface to a user, receive user input from the patient display device, and transform the user input into the control commands for wireless transmission to the implantable medical device.

According to one embodiment, the wireless communication unit comprises a wireless transceiver for wireless transmission of control commands to the implantable medical device, and wireless transmission of the control interface as the remote display portal to the patient display device.

According to one embodiment, the wireless communication unit comprises a first wireless transceiver for wireless transmission of control commands to the implantable medical device, and a second wireless transceiver for wireless transmission of the control interface to the patient display device.

According to one embodiment, the wireless communication unit is configured for wireless communication with the patient display device using a standard network protocol.

According to one embodiment, the wireless communication unit is configured for wireless communication with the implantable medical device using a proprietary network protocol.

According to one embodiment, the wireless communication unit comprises a Bluetooth transceiver.

According to one embodiment, at least one of the first and second wireless transceiver comprises a Bluetooth transceiver.

According to one embodiment, the wireless communication unit comprises a UWB transceiver.

According to one embodiment, at least one of the first and second wireless transceiver comprises a UWB transceiver.

According to one embodiment, the wireless communication unit comprises at least one first wireless transceiver configured for communication with the implantable medical device using a first network protocol, for determining a distance between the patient external device and the implantable medical device, and at least one second wireless transceiver configured for communication with the implantable medical device using a second network protocol, for transferring data between the patient external device and the implantable medical device.

According to one embodiment, the first wireless transceiver is configured for transcutaneous energy transfer for at least one of: powering an energy consuming component of the implantable medical device and charging an implantable energy storage unit.

According to one embodiment, the standard network protocol is one from the list of Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the first wireless transceiver is less than a communication range of the second wireless transceiver.

According to one embodiment, at least one of
the patient external device is configured to authenticate the implantable medical device if a distance between the patient external device and the implantable medical device is less than a predetermined threshold value,
the patient external device is configured to be authenticated by the implantable medical device if a distance between the patient external device and the implantable medical device is less than a predetermined threshold value,
the patient external device is configured to authenticate the patient display device if a distance between the patient external device and the patient display device is less than a predetermined threshold value, and
the patient external device is configured to be authenticated by the implantable medical device if a distance between the patient external device and the patient display device is less than a predetermined threshold value.

According to one embodiment, the patient external device is configured to allow the transfer of data between at least one of the patient external device and the implantable medical device, and the patient external device and the patient display device, on the basis of the authentication.

According to one embodiment, the computing unit is configured to encrypt at least one of the control interface and the control commands.

According to one embodiment, the implantable medical device comprises at least one of an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
a device to stimulate the brain for a several position to a focused point,
an artificial stomach replacing the function of the natural stomach,
an implant configured for adjusting the position of a female's urinary tract or bladder neck,
an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A patient display device for communication with a patient remote external device for communication with an implantable medical device is provided. The patient display device comprises a wireless communication unit configured for wirelessly receiving an implant control interface as a remote display portal from the patient remote external device and configured for wirelessly transmitting implant control user input to the patient remote external device, a display for displaying the received implant control interface, and an input device for receiving implant control input from the user.

According to one embodiment, the patient display device further comprises an auxiliary wireless communication unit. The auxiliary wireless communication unit is configured to be disabled to enable at least one of: wirelessly receiving the implant control interface as the remote display portal from the patient remote external device, and wirelessly transmitting implant control user input to the patient remote external device.

According to one embodiment, the wireless communication unit is configured for wireless communication with the patient remote external device using a standard network protocol. The standard network protocol may be one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the wireless communication unit is configured for wireless communication with the patient remote external device using a proprietary network protocol.

According to one embodiment, the wireless communication unit comprises a Bluetooth transceiver.

According to one embodiment, the wireless communication unit comprises a UWB transceiver.

According to one embodiment, a communication range of the wireless communication unit is less than a communication range of the auxiliary wireless communication unit.

According to one embodiment, the patient display device is configured to authenticate the patient remote external device if a distance between the patient display device and the patient remote external device is less than a predetermined threshold value, or to be authenticated by the patient remote external device if a distance between the patient display device and the patient remote external device is less than a predetermined threshold value.

According to one embodiment, the patient display device is configured to allow the transfer of data between the patient display device and the patient remote external device on the basis of the authentication.

According to one embodiment, the patient display device is a wearable external device or a handset.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device and an implantable medical device, when implanted, is provided. The communication system comprises: a patient display device,
a server, and a patient remote external device. The patient display device comprises a wireless communication unit configured for wirelessly receiving an implant control interface as a remote display portal being provided by the patient remote external device. The wireless communication unit is further configured for wirelessly transmitting implant control user input to the server, destined for the patient remote external device. The system further comprises a display for displaying the received remote display portal, and an input device for receiving implant control input from the user, wherein the patient remote external device comprises a wireless communication unit configured for wireless transmission of control commands to the implantable medical device, and a computing unit. The computing unit is configured for running a control software for creating the control commands for the operation of the implantable medical device, transmitting a control interface to the patient display device, receiving implant control user input generated at the patient display device, from the server, and transforming the user input into the control commands for wireless transmission to the implantable medical device.

According to one embodiment, the computing unit is configured to encrypt at least one of the control interface and the control commands.

According to one embodiment, the patient display device is configured to encrypt the user input.

According to one embodiment, the server is configured to encrypt at least one of the user input received from the patient display device and the control interface received from the patient remote external device.

According to one embodiment, the computing unit is configured to encrypt the control interface and the patient display device is configured to decrypt the encrypted control interface.

According to one embodiment, the server is configured to act as a router, transferring the encrypted control interface from the patient remote external device to the patient display device without decryption.

According to one embodiment of the communication system or patient display device the implantable medical device comprises at least one of:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
   an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
a device to stimulate the brain for a several position to a focused point,
an artificial stomach replacing the function of the natural stomach,
an implant configured for adjusting the position of a female's urinary tract or bladder neck,
an implant configured for stimulating the ampulla vas deference and creating temporary constriction.

According to one embodiment, the communication system further comprises a server. The server may comprise a wireless communication unit configured for wirelessly receiving an implant control interface received from the patient remote external device and wirelessly transmitting the implant control interface as a remote display portal to the patient display device. The wireless communication unit is further configured for wirelessly receiving implant control user input from a patient EID external device and wirelessly transmitting the implant control user input to the patient display device.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A patient display device for communication with a patient external device for communication with an implantable medical device, when implanted, is provided. The patient display device comprises a wireless communication unit, a display, and an input device for receiving implant control input from the user. The patient display device is configured to run a first application for wireless communication with a server and/or DDI, and run a second application for wireless communication with the patient external device for transmission of the implant control input to a remote display portal of the patient external device for the communication with the implantable medical device, wherein the second application is configured to be accessed through the first application. The patient display device comprises a first log-in function and a second log-in function, wherein the first log-in function gives the user access to the first application and wherein the first and second log-in function in combination gives the user access to the second application. The first log-in function may be configured to use at least one of a password, pin code, fingerprint, voice and face recognition. A second log-in function within the first application may be configured to use a private key from the user to authenticate, for a defined time period, a second hardware key of the patient external device.

According to one embodiment, the first log-in is a PIN-based log-in.

According to one embodiment, at least one of the first and second log-in is a log-in based on a biometric input or a hardware key.

According to one embodiment, the patient display device further comprises an auxiliary wireless communication unit, and wherein the auxiliary wireless communication unit is configured to be disabled to enable wireless communication with the patient external device.

According to one embodiment, the patient display device is configured to wirelessly receive an implant control interface as a remote display portal from the patient external device to be displayed on the display.

According to one embodiment, the wireless communication unit is configured for wireless communication with the patient external device using a standard network protocol.

According to one embodiment, the wireless communication unit is configured for wireless communication with the patient external device using a proprietary network protocol.

According to one embodiment, the wireless communication unit is configured for wireless communication with the patient external device using a first network protocol and with the server using a second network protocol.

According to one embodiment, the wireless communication unit is configured for wireless communication with the patient external device using a first frequency band and with the server using a second frequency band.

According to one embodiment, the wireless communication unit comprises a Bluetooth transceiver.

According to one embodiment, the wireless communication unit comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, a communication range of the wireless communication unit is less than a communication range of the auxiliary wireless communication unit.

According to one embodiment, the wireless communication unit comprises a first wireless transceiver for communication with the patient external device and a second wireless transceiver for communication with the server.

According to one embodiment, the second wireless transceiver is configured to be disabled to enable wireless communication using the first wireless transceiver.

According to one embodiment, the patient display device is configured to authenticate the patient external device if a distance between the patient display device and the patient external device is less than a predetermined threshold value, or to be authenticated by the patient external device if a distance between the patient display device and the patient external device is less than a predetermined threshold value.

According to one embodiment, the patient display device is configured to allow the transfer of data between the patient display device and the patient external device on the basis of the authentication.

According to one embodiment, the patient display device is a wearable external device or a handset.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, and an error.

According to one embodiment, the patient display device is configured to encrypt the user input.

According to one embodiment, the display is configured to encrypt the user input for decryption by the implantable medical device.

According to one embodiment, the patient display device is configured to decrypt the control interface received from the patient external device, for displaying the control interface on the display.

According to one embodiment, at least one of the first and second application is configured to receive data from an auxiliary external device and present the received data to the user.

According to one embodiment, at least one of the first and second application is configured to receive data from an auxiliary external device comprising a scale for determining the weight of the user.

According to one embodiment, at least one of the first and second application is configured to receive data related to the weight of the user from an auxiliary external device comprising a scale.

According to one embodiment, the patient display device is configured to: wirelessly transmit the data related to the weight of the user to the patient external device, or wirelessly transmit an instruction derived from the data related to the weight of the user, or wirelessly transmit an instruction derived from a combination of the data related to the weight of the user and the implant control input received from the user.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device and an implantable medical device, when implanted, is provided. The communication system comprises a patient display device,
a server or DDI, and a patient remote external device. The patient display device comprises a wireless communication unit configured for wirelessly receiving an implant control interface as a remote display portal from the patient remote external device, the wireless communication unit further being configured for wirelessly transmitting implant control user input to the patient remote external device, a display for displaying the received implant control interface as a remote display portal, and an input device for receiving implant control input from the user. The patient display device is configured to run a first application for wireless communication with the server, and to run a second application for wireless communication with the patient remote external device for transmission of the implant control input to the remote display portal of the patient remote external device for the communication with the implantable medical device. The patient remote external device comprises a wireless communication unit configured for wireless transmission of control commands based on the implant control input to the implantable medical device and configured for wireless communication with the patient display device.

According to one embodiment, the patient display device comprises a first log-in function and a second log-in function, and wherein the first log-in function gives the user access to the first application and wherein the first and second log-in function in combination gives the user access to the second application.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, or an error.

According to one embodiment, the patient display device is configured to encrypt the user input.

According to one embodiment, the display is configured to encrypt the user input for decryption by the implantable medical device.

According to one embodiment, the patient remote external device is configured to act as a router, transferring the encrypted user input from the patient display device to the implantable medical device without decryption.

According to one embodiment, the patient remote external device is configured to encrypt at least one of the control interface and the control commands.

According to one embodiment, the patient remote external device is configured to encrypt the control interface and wherein the patient display device is configured to decrypt the encrypted control interface.

A computer program product is provided, configured to run in a patient display device comprising a wireless communication unit, a display for displaying the received implant control interface as a remote display portal, and an input device for receiving implant control input from a user. The computer program product comprises:
a first application for communication with a server or DDI,
a second application for communication with an patient remote external device for transmission of the implant control input via the remote display portal of the patient remote external device for the communication with an implantable medical device, wherein the second application is configured to be accessed through the first application,
a first log-in function using at least one of a password, pin code, fingerprint, or face recognition, and
a second log-in function within the first application, using a private key from the user to authenticate for a defined time period a second hardware key of the patient remote external device. The first log-in function gives the user access to the first application and the first and second log-in function in combination gives the user access to the second application.

According to one embodiment, the second application is configured to receive data related to a parameter of the implanted medical device.

According to one embodiment, the second application is configured to receive data related to a sensor value received from the implanted medical device.

According to one embodiment, the second application is configured to receive data related to a parameter related to at least one of: a battery status, a temperature, a time, or an error.

According to one embodiment of the communication system, patient display device or computer program product, the implantable medical device comprises at least one of: an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries. an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A communication system for enabling communication between a patient display device, a patient external device, a server and an implantable medical device, is provided. The communication system comprises a server,
a patient display device, a patient external device, and an implantable medical device. The patient display device comprises a wireless communication unit for wirelessly communicating with at least one of the patient external device and the server, a display, and an input device for receiving input from the user. The patient external device comprises a wireless communication unit configured for wireless transmission of control commands to the implantable medical device and configured for wireless communication with at least one of the patient display device and the server. Further, the server comprises a wireless communication unit configured for wireless communication with at least one of the patient display device and the patient external device, wherein the implantable medical device comprises a wireless communication unit configured for wireless communication with the patient external device. The implantable medical device further comprises an encryption unit and is configured to: encrypt data destined for the server, transmit the data to the server via the patient external device, wherein the patient external device acts as a router transferring the data without full decryption. In an example, the implantable medical device comprises an encryption unit and is configured to: encrypt data destined for the patient display device, transmit the data to the patient display device via the patient external device, wherein the patient external device acts as a router transferring the data without full decryption. In an example, the server comprises an encryption unit and is configured to: encrypt data destined for the implantable medical device, transmit the data to the implantable medical device via the patient external device, wherein the patient external device acts as a router transferring the data without full decryption, In an example, the server comprises an encryption unit and is configured to: encrypt data destined for the implantable medical device, transmit the data to the implantable medical device via the patient display device and the patient external device, wherein the patient display device and the patient external device acts as a router transferring the data without full decryption. In an example, the patient display device comprises an encryption unit and is configured to: encrypt data destined for the implantable medical device, transmit the data to the implantable medical device via the patient external device, wherein the patient external device acts as a router transferring the data without full decryption. In an example, the patient display device comprises an encryption unit and is configured to: encrypt data destined for the implantable medical device, transmit the data to the implantable medical device via the server and the patient external device, wherein the server and the patient external device acts as a router transferring the data without full decryption.

According to one embodiment, the patient display device is configured to wirelessly receive an implant control interface from the patient external device to be displayed on the display.

According to one embodiment, at least two of: the wireless communication unit of the server, the wireless communication unit of the patient display device, the wireless communication unit of the patient external device, and the wireless communication unit of the implantable medical device, are configured for wireless communication using a standard network protocol.

According to one embodiment, wherein at least two of: the wireless communication unit of the server, the wireless communication unit of the patient display device, the wireless communication unit of the patient external device, and the wireless communication unit of the implantable medical device, are configured for wireless communication using a proprietary network protocol.

According to one embodiment, the wireless communication unit of the patient external device is configured to use a first network protocol for communication with the implantable medical device and use a second network protocol for communication with the server, or use a first network protocol for communication with the implantable medical device and use a second network protocol for communication with the patient display device.

According to one embodiment, the wireless communication unit of the patient external device is configured to use a first frequency band for communication with the implantable medical device and use a second frequency band for communication with the server, or use a first frequency band for communication with the implantable medical device and use a second frequency band for communication with the patient display device.

According to one embodiment, the wireless communication unit of the patient display device is configured to use a first network protocol for communication with the patient external device and use a second network protocol for communication with the server.

According to one embodiment, the wireless communication unit of the patient display device is configured to use a first frequency band for communication with the patient external device and use a second frequency band for communication with the server.

According to one embodiment, the wireless communication unit of the server is configured to use a first network protocol for communication with the patient external device and use a second network protocol for communication with the patient display device.

According to one embodiment, the wireless communication unit of the server is configured to use a first frequency band for communication with the patient external device and use a second frequency band for communication with the patient display device.

According to one embodiment, the wireless communication unit of at least one of the server, the patient display device, the patient external device, and the implantable medical device comprises a Bluetooth transceiver.

According to one embodiment, the wireless communication unit of at least one of the server, the patient display device, the patient external device, and the implantable medical device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the wireless communication unit of the patient external device comprises a first wireless transceiver for wireless communication with the implantable medical device, and a second wireless transceiver for wireless communication with the server, and wherein the second wireless transceiver has a longer effective range than the first wireless transceiver.

According to one embodiment, the wireless communication unit of the patient external device comprises a first wireless transceiver for wireless communication with the implantable medical device, and a second wireless transceiver for wireless communication with the patient display device, and wherein the second wireless transceiver has a longer effective range than the first wireless transceiver.

According to one embodiment, the wireless communication unit of the patient display device comprises a first wireless transceiver for wireless communication with the patient external device, and a second wireless transceiver for wireless communication with the server, and wherein the second wireless transceiver has a longer effective range than the first wireless transceiver.

According to one embodiment, the second wireless transceiver has an effective range being one of: 2 times, 4 times, 8 times 20 times, 50 times or 100 times longer than the first wireless transceiver.

According to one embodiment, the second wireless transceiver is configured to be disabled to enable wireless communication using the first wireless transceiver.

According to one embodiment, at least one of:
the patient display device is configured to authenticate the patient external device if a distance between the patient display device and the patient external device is less than a predetermined threshold value,
the patient display device is configured to be authenticated by the patient external device if a distance between the patient display device and the patient external device is less than a predetermined threshold value,
the patient display device is configured to authenticate the implantable medical device if a distance between the patient display device and the implantable medical device is less than a predetermined threshold value,
the patient display device is configured to be authenticated by the implantable medical device if a distance between the patient display device and the implantable medical device is less than a predetermined threshold value,
the patient external device is configured to authenticate the patient display device if a distance between the patient external device and the patient display device is less than a predetermined threshold value,
the patient external device is configured to be authenticated by the patient display device if a distance between the patient external device and the patient display device is less than a predetermined threshold value,
the patient external device is configured to authenticate the implantable medical device if a distance between the patient external device and the implantable medical device is less than a predetermined threshold value, and
the patient external device is configured to be authenticated by the implantable medical device if a distance between the patient external device and the implantable medical device is less than a predetermined threshold value.

According to one embodiment, the patient display device is configured to allow the transfer of data between the patient display device and the patient external device on the basis of the authentication.

According to one embodiment, the patient external device is configured to allow the transfer of data between the patient display device and the patient external device on the basis of the authentication.

According to one embodiment, the patient external device is configured to allow the transfer of data between the patient external device and the implantable medical device on the basis of the authentication.

According to one embodiment, the patient display device is a wearable patient external device or a handset.

According to one embodiment, the data encrypted by the implantable medical device is related to at least one of a battery status, a temperature, a time, or an error.

A server for use in the communication system according to any one of the above embodiments is provided.

A patient display device for use in the communication system according to any one of the above embodiments is provided.

A patient external device for use in the communication system according to any one of the above embodiments is provided.

An implantable medical device for use in the communication system according to any one of the above embodiments is provided.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system configured for changing pre-programmed treatment settings of an implantable medical device, when implanted in a patient, from a distant remote location in relation to the patient, is provided. The system comprises at least one health care provider, HCP, EID external device, and a HCP private key device. HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication. The HCP EID external device comprises at least one of: a reading slot or comparable for the HCP private key device, a RFID communication, and other close distance wireless activation communication or electrical direct contact. The HCP EID external device further comprises at least one wireless transceiver configured for communication with a data infrastructure server, DDI, through a first network protocol. Further, the system comprises a data infrastructure server, DDI, adapted to receive command from said HCP EID external device and to relay the received command without modifying said command to a patient EID external device, wherein the DDI comprises one wireless transceiver configured for communication with said patient external device, and a patient EID external device adapted to receive the command relayed by the DDI, further adapted to send this command to the implanted medical device, further adapted to receive a command from the HCP EID external device via the DDI to change said pre-programmed treatment settings of the implanted medical device, and further adapted to be activated and authenticated and allowed to perform said command by the patient providing a patient private key device adapted to be provided to the patient EID external device by the patient via at least one of: a reading slot or comparable for the patient private key device, a RFID communication or other close distance wireless activation communication or electrical direct contact. The patient EID external device comprises at least one of a reading slot or comparable for the HCP private key device, a RFID communication, and other close distance wireless activation communication or electrical direct contact. The patient EID external device further comprises at least one wireless transceiver configured for communication with the implanted medical device through a second network protocol. Further, the implanted medical device is configured to treat the patient or perform a bodily function.

According to one embodiment, at least one of the patient private key device or HCP private key device comprises a hardware key.

According to one embodiment, the private key device is at least one of, a smartcard, a key-ring device, a watch an arm or wrist band a neckless or any shaped device.

According to one embodiment of the system, at least two of: the HCP EID external device, the patient EID external device, the HCP private key device, the patient private key device, and the DDI are configured for wireless communication using a standard network protocol.

According to one embodiment, at least two of: the HCP EID external device, the patient EID external device, the HCP private key device, the patient private key device, and the DDI are configured for wireless communication using a proprietary network protocol.

According to one embodiment, the patient EID external device is configured to use a first network protocol for communication with the implantable medical device and use a second network protocol for communication with the DDI.

According to one embodiment, the patient EID external device is configured to use a first frequency band for communication with the implantable medical device and use a second frequency band for communication with the DDI.

According to one embodiment, the DDI is configured to use a first frequency band for communication with the patient EID external device and a second frequency band for communication with the patient private key device.

According to one embodiment, at least one of the HCP EID external device, the patient EID external device, the HCP private key device, the patient private key device and the DDI comprises a Bluetooth transceiver.

According to one embodiment, at least one of the HCP EID external device, the patient EID external device, the HCP private key device, the patient private key device and the DDI comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the DDI, and wherein the second wireless transceiver has longer effective range than the first wireless transceiver.

According to one embodiment, the patient private key device comprises a first wireless transceiver for wireless communication with the HCP EID external device, and a second wireless transceiver for wireless communication with the DDI, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is configured to allow transfer of data between the EID external device and the implantable medical device on the basis of an authentication of the patient EID external device.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for changing pre-programmed treatment settings of an implantable medical device, when implanted in a patient, by a health care provider, HCP, in the physical presence of the patient. The system comprises at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing an HCP private key device comprising a HCP private key. The HCP private key device comprises at least one of: a smart card, a keyring device, a watch, an arm or wrist band, a necklace, and any shaped device. The HCP EID external device is adapted to be involved in at least one of: receiving information from the implant, receiving information from a patient remote external device, actuating the implanted medical device, changing pre-programmed settings, and updating software of the implantable medical device, when implanted. The HCP EID external device is further adapted to be activated, authenticated, and allowed to perform said command also by the patient. The system further comprises a patient private key device comprising a patient private key, wherein the patient private key device comprising at least one of: a smart card, a keyring device, a watch, an arm or wrist band, a necklace, and any shaped device. The HCP private key and the patient private key are required for performing said actions by the HCP EID external device to at least one of: receive information from the implant, to receive information from a patient remote external device, to actuate the implanted medical device, to change pre-programmed settings, and to update software of the implantable medical device, when the implantable medical device is implanted.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of reading slot or comparable for the HCP private key device,
a RFID communication and a close distance wireless activation communication unit, or electrical direct contact.

According to one embodiment, the HCP EID external device is adapted to receive a command from a HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, at least two of: the HCP EID external device, the patient EID external device, the HCP private key device, and the patient private key device, are configured for wireless communication using a standard network protocol.

According to one embodiment, at least two of: the HCP EID external device, the patient EID external device, the HCP private key device, and the patient private key device, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the HCP EID external device, the patient EID external device, the HCP private key device, and the patient private key device comprises a Bluetooth transceiver.

According to one embodiment, at least one of the HCP EID external device, the patient EID external device, the HCP private key device, and the patient private key device comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is configured to allow transfer of data between the EID external device and the implantable medical device on the basis of an authentication of the patient EID external device.

According to one embodiment, the patient EID external device is a wearable patient external device or a handset.

According to one embodiment, the data encrypted by the implantable medical device is related to at least one of: a battery status, a temperature, a time, or an error.

A system is provided, configured to change pre-programmed and pre-selected treatment actions of an implantable medical device, when implanted in a patient, by command from the patient. The system comprises an implantable medical device, a patient remote external device, a wireless transceiver configured for communication with the implantable medical device, when the medical device is implanted, through a second network protocol, and a remote display portal. The remote display portal is configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device.

According to one embodiment, the wireless transceiver, the remote display portal, and the remote display portal are comprised in the patient remote external device.

According to one embodiment, the system further comprises the patient display device, which may comprise a supporting application, a display which hosts the Remote Display Portal, and a patient display device private key.

According to one embodiment, the remote display portal is capable of generating a command to be signed by the patient display device private key.

According to one embodiment, the patient remote external device is adapted to accept input from the patient via said patient display device through its remote display portal.

According to one embodiment, the patient remote external device comprises a graphical user interface arranged on a touch-responsive display exposing buttons to express actuation functions of the implanted medical device.

According to one embodiment, the system is configured to allow the patient to actuate the implant at home through the patient remote external device by means of an authorization granted by a patient private key.

According to one embodiment, the patient private key comprises at least one of: a smart card, a keyring device, a watch, an arm or wrist band, a necklace, and any shaped device.

According to one embodiment, the system is configured to allow the patient to actuate the implantable medical device, when implanted, at home through the patient remote external device, using an authorization granted by the patient private key.

According to one embodiment, system further comprises a patient EID external device comprising at least one of: a reading slot or comparable for the patient private key device, a RFID communication, and a close distance wireless activation communication, or electrical direct contact.

According to one embodiment, the patient EID external device is adapted to be synchronized with the patient remote external device.

According to one embodiment, the patient EID external device further comprises at least one of: a wireless transceiver configured for communication with the patient, a remote external device, and a wired connector for communication with the patient remote external device.

According to one embodiment, the patient EID external device is adapted to generate an authorization to be signed by the patient private key to be installed into at least one of: the patient remote external device through the patient EID external device, and the implantable medical device.

According to one embodiment, the system comprises a patient display device comprising a supporting application capable of displaying the remote display portal with content delivered from the patient remote external device.

According to one embodiment, the remote display portal and patient remote external device are adapted to expose buttons to express the will to actuate the functions of the implanted medical device by the patient through the patient remote external device.

According to one embodiment, the patient display device comprises at least one of: a display which hosts the remote display portal, and a patient display device private key.

According to one embodiment, the remote display portal is capable of generating a command to be signed by the patient private key.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for providing information from an implantable medical device, when implanted in a patient, from a distant remote location in relation to the patient. The system comprises at least one patient EID external device adapted to receive information from the implant, adapted to send such information further on to a server or dedicated data infrastructure, DDI, further adapted to be activated and authenticated and allowed to receive said information by the implanted medical device by the patient providing a private key. Further, the system comprises a patient private key device comprising the private key adapted to be provided to the patient EID external device via at least one of: a reading slot or comparable for the patient private key device, a RFID communication or other close distance wireless activation communication or direct electrical connection. The patient EID external device comprises at least one of: a reading slot or comparable for the patient private key device, an RFID communication, and other close distance wireless activation communication or direct electrical contact. Further, the patient EID external device comprises at least one wireless transceiver configured for communication with the DDI, through a first network protocol.

According to one embodiment, the at least one patient EID external device is adapted to receive information from the implant, through a second network protocol.

According to one embodiment, the system comprises the DDI, wherein the DD1 is adapted to receive information from said patient EID external device, and wherein the DDI comprises a wireless transceiver configured for communication with said patient EID external device.

According to one embodiment, the patient EID external device is adapted to receive a command relayed by the DDI, to further send the command to the implanted medical device to change said pre-programmed treatment settings of the implanted medical device, and further adapted to be activated and authenticated and allowed to perform said command by the patient providing the patient private key.

According to one embodiment, the patient private key device is adapted to provide the patient private key to the patient EID external device by the patient via at least one of; a reading slot or comparable for the patient private key device, an RFID communication or other close distance wireless activation communication, or electrical direct contact.

According to one embodiment, the patient EID external device comprises at least one of: a reading slot or comparable for the HCP private key device, a RFID communication, and other close distance wireless activation communication, or direct electrical contact.

According to one embodiment, the patient EID external device further comprising at least one wireless transceiver configured for communication with the implanted medical device through a second network protocol.

According to one embodiment, the system comprises the implantable medical device, which may be adapted to, when implanted, treat the patient or perform a bodily function.

According to one embodiment, the patient private key comprises at least one of: a smart card, a keyring device, a watch, an arm band or wrist band, a necklace, and any shaped device.

According to one embodiment, at least two of: the patient EID external device, the IDD, and the patient private key device, are configured for wireless communication using a standard network protocol.

According to one embodiment, at least two of: the patient EID external device, the IDD, and the patient private key device, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the patient EID external device, the patient private key device and the IDD comprises a Bluetooth transceiver.

According to one embodiment, at least one of the patient EID external device, the patient private key device and the IDD comprises a UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of a new private key device, wherein the HCP or HCP admin have such master private key device adapted to able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, comprising, an implantable medical device adapted to, when implanted in a patient, to communicate with an external device, the external device comprising at least one of a patient remote external device or a patient EID external device. The system further comprises the patient EID external device adapted to communicate with and send commands to the implantable medical device when implanted, to change pre-programmed settings, and a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact. Further the system comprises a data infrastructure server, DDI, adapted to send commands to the patient EID external device for further transport to the implanted medical device, to inactivate the authority and authenticating function of the patient private key.

According to one embodiment, the at least one patient remote external device comprises a patient remote external device private key, wherein the DDI via the patient EID external device is able to inactivate the authority and authenticating function of the patient remote external device, thereby inactivating the patient remote external device.

According to one embodiment, the patient EID external device comprises at least one wireless transceiver configured for communication with the DD1 via a first network protocol.

According to one embodiment, the system comprises the DDI, wherein the DDI is adapted to receive command from a HCP EID external device, and to send the received command to the patient EID external device, wherein the DDI comprises a wireless transceiver configured for communication with said patient external device.

According to one embodiment, the patient EID external device is adapted to receive the command from the DDI, wherein the command originates from a health care provider, HCP, and wherein the patient EID is adapted to inactivate the patient private key and to send the command to the implanted medical device.

According to one embodiment, the patient EID external device is adapted to receive the command from the DDI, wherein the command originates from a health care provider, HCP, wherein the patient EID external device is adapted to receive the command from the HCP via the DDI to inactivate the patient remote external device comprising a patient remote external device private key, and wherein the patient EID external device is further adapted to send this command to the implanted medical device.

According to one embodiment, the patient EID external device further comprises at least one wireless transceiver configured for communication with the implanted medical device through a second network protocol.

According to one embodiment, at least one of the patient private key and a patient remote external device private key comprises a hardware key.

According to one embodiment, the private key device is at least one of, a smartcard, a key-ring device, a watch an arm or wrist band a neckless or any shaped device.

According to one embodiment, at least two of: the patient remote external device, the patient EID external device, the patient private key device, and the DDI, are configured for wireless communication using a standard network protocol.

According to one embodiment, wherein at least two of: the patient remote external device, the patient EID external device, the patient private key device, and the DDI, are configured for wireless communication using a proprietary network protocol.

According to one embodiment, the patient EID external device is configured to use a first network protocol for communication with the implantable medical device and use a second network protocol for communication with the patient private key device.

According to one embodiment, the patient EID external device is configured to use a first frequency band for communication with the implantable medical device and use a second frequency band for communication with the patient private key device.

According to one embodiment, at least one of the patient remote external device, the patient EID external device, the patient private key device, and the DDI, comprise a Bluetooth transceiver.

According to one embodiment, at least one of the patient remote external device, the patient EID external device, the patient private key device, and the DDI, comprise an UWB transceiver.

According to one embodiment, the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to one embodiment, the patient EID external device comprises a first wireless transceiver for wireless communication with the implantable medical device, and a second wireless transceiver for wireless communication with the patient private key device, and wherein the second wireless transceiver has longer effective range than the first wireless transceiver.

According to one embodiment, the second wireless transceiver has an effective range being one of: 2 times, 4 times, 8 time, 20 times, 50 times or 100 times longer than the effective range of the first wireless transceiver.

According to one embodiment, the second wireless transceiver is configured to be disabled to enable wireless communication using the first wireless transceiver.

According to one embodiment, the patient EID external device is a wearable patient external device or a handset.

According to one embodiment, the data encrypted by the implantable medical device is related to at least one of: a battery status, a temperature, a time, or an error.

According to one embodiment, the system comprises a master private key device configured to allow issuance of new private key device, wherein the HCP or HCP admin have such master private key device adapted to be able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallows solid food or is drinking fluid, wherein said food sensor is connected to the control unit of a medical device to cause an action to stretch the stomach after a determined amount of food intake.

A system is provided, configured for changing pre-programmed treatment settings in steps of an implantable medical device, when implanted in a patient, by a health care provider, HCP, either in the physical presence of the patient or remotely with the patient on distance. The system comprises at least one HCP EID external device adapted to receive a command directly or indirectly from the HCP to change said pre-programmed treatment settings in steps of the implantable medical device, when implanted. The HCP EID external device is further adapted to be activated, authenticated, and allowed to perform said command by the HCP providing a HCP private key device comprising a HCP private key. The HCP private key comprises at least one of: a smart card, a keyring device, a watch, an arm or wrist band, a necklace, and any shaped device. The system further comprises a patient private key device comprising a patient private key, comprising at least one of: a smart card, a keyring device, a watch, an arm or wrist band, a necklace, and any shaped device. Both the HCP and patient private key is required for performing said action by the HCP EID external device to change the pre-programmed settings in the implant and to update software of the implantable medical device, when the implantable medical device is implanted. The patient private key is adapted to activate, be authenticated, and allowed to perform said command provided by the HCP, either via the HCP EID external device or when the action is performed remotely via a patient EID external device.

According to one embodiment, the system comprises a master private key device that allow issuance of new private key device wherein the HCP or HCP admin have such master private key device adapted to be able to replace and pair a new patient private key device or HCP private key device into the system, through the HCP EID external device.

According to one embodiment, the patient remote external device and the patient EID external device are an integrated unit.

According to one embodiment, the HCP dedicated device and the HCP EID external device are an integrated unit.

According to one embodiment, the system further comprises a measurement device or sensor adapted to deliver a measurement to at least one of the DDI, patent EID external device and a patient display device.

According to one embodiment, the system further comprises a food sensor adapted to measure at least if the patient swallows solid food or is drinking fluid, wherein said food sensor is configured to be connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

A system is provided, configured for changing pre-programmed treatment settings in steps of an implantable medical device, when implanted in a patient, by a health care provider, HCP, with the patient on remote on distance. The system comprises at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP. The action by the HCP EID external device to change pre-programmed settings in the implant and to update software of the implantable medical device, when the implantable medical device is implanted, is adapted to be authenticated by a HCP private key device and a patient private key device.

According to one embodiment, the HCP private key device comprising a HCP private key, comprising at least one of a smart card, a keyring device, a watch, an arm or wrist band, a necklace, and any shaped device.

According to one embodiment, the patient private key device comprises a patient private key, comprising at least one of a smart card, a keyring device, a watch, an arm or wrist band, a necklace, and any shaped device.

According to one embodiment, the patient private key is adapted to activate, be authenticated, and allowed to perform said command provided by the HCP, either via the HCP EID external device or when the action is performed remotely via a patient EID external device.

According to one embodiment, the system further comprises a dedicated data infrastructure, DDI, the patient EID external device, and the HCP EID external device, wherein the communication between the patient EID external device and the HCP EID external device is performed via the DDI.

According to one embodiment, the system comprises a master private key device that allows issuance of new private key device wherein the HCP or HCP admin have such master private key device adapted to be able to replace and pair a new patient private key device or HCP private key device into the system.

According to one embodiment, the patient remote external device and the patient EID external device are an integrated unit.

According to one embodiment, the HCP dedicated device and the HCP EID external device are an integrated unit.

According to one embodiment, the system further comprises a measurement device or sensor adapted to deliver a measurement to at least one of the DDI, patent EID external device and patient display device.

According to one embodiment, the system comprises a food sensor, adapted to measure at least if the patient swallow solid food or is drinking fluid, wherein said food sensor is connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of: a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

A system is provided, which is configured for changing pre-programmed treatment settings of an implantable medical device, when implanted in a patient, from a distant remote location in relation to the patient. The system comprises at least one health care provider, HCP, external device adapted to receive a command from the HCP to change said pre-programmed treatment settings of an implanted medical device. The HCP external device is further adapted to be activated and authenticated and allowed to perform said command by the HCP providing a HCP private key device adapted to be provided to an HCP EID external device via at least one of; a reading slot or comparable for the HCP private key device, a RFID communication or other close distance wireless activation communication. The HCP EID external device comprises at least one of: a reading slot or comparable for the HCP private key device, a RFID communication, and other close distance wireless activation communication or electrical direct contact. The HCP EID external device further comprises at least one wireless transceiver configured for communication with a patient EID external device, through a first network protocol. The system comprises the patient EID external device, the patient EID external device being adapted to receive command from said HCP external device, and to relay the received command without modifying said command to the implanted medical device. The patient EID external device comprises one wireless transceiver configured for communication with said patient external device, wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.

According to one embodiment, at least one of the patient private key device or HCP private key device comprises a hardware key.

According to one embodiment, the private key device is at least one of, a smartcard, a key-ring device, a watch an arm or wrist band a neckless or any shaped device.

According to one embodiment, the system comprises a master private key device that allow issuance of new private key device wherein the HCP or HCP admin have such master private key device adapted to be able to replace and pair a new patient private key device or HCP private key device into the system, through the HCP EID external device.

According to one embodiment, the patient remote external device and the patient EID external device is an integrated unit.

According to one embodiment, the HCP dedicated device and the HCP EID external device are an integrated unit.

According to one embodiment, the system comprises a measurement device or sensor adapted to deliver a measurement to at least one of the DDI, patent EID external device and a patient display device.

According to one embodiment, the system comprises a food sensor, adapted to measure at least if the patient swallow solid food or is drinking fluid, wherein said food sensor is connected to the control unit of a medical device to cause an action to stretch the stomach after a determined amount of food intake.

According to one embodiment, the HCP EID external device further comprises a wireless transceiver configured for communication with the implanted medical device through a second network protocol.

According to one embodiment, the HCP private key device is adapted to be provided to the at least one HCP external device via at least one of; a reading slot or comparable for the HCP private key device, a RFID communication, and a close distance wireless activation communication unit, or electrical direct contact.

According to one embodiment, the HCP EID external device comprises at least one of: reading slot or comparable for the HCP private key device, a RFID communication, and a close distance wireless activation communication unit, or electrical direct contact.

According to one embodiment, the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.

According to one embodiment, the HCP EID external device and the HCP private key device are configured for wireless communication using a standard network protocol.

According to one embodiment, the HCP EID external device and the HCP private key device are configured for wireless communication using a proprietary network protocol.

According to one embodiment, the HCP EID external device is configured to use a first network protocol for communication with the implantable medical device and use a second network protocol for communication with the HCP private key device.

According to one embodiment, the HPC EID external device is configured to use a first frequency band for communication with the implantable medical device and use a second frequency band for communication with the HCP private key device.

According to one embodiment, at least one of the HCP EID external device and the HCP private key device comprises a Bluetooth transceiver.

According to one embodiment, at least one of the HCP EID external device and the HCP private key device comprises a UWB transceiver.

### Aspect 330SE eHealth General Communication Housing

An external device configured for communication with an implantable medical device when implanted in a patient is further provided. The external device comprising, a display device, a housing unit configured to mechanically, disconnectably connect to the display device, the housing comprises a first communication unit for receiving communication from the display device, and a second communication unit for wirelessly transmitting communication to the implantable medical device.

According to one embodiment, the external device comprises a handheld electronic device.

According to one embodiment, the external device is configured for communicating with the implantable medical device for changing the operational state of an implantable medical device. The advantage of the embodiment is that the operational state of the implantable medical device can be changed remotely.

According to one embodiment, the first communication unit is a wireless communication unit for wireless communication with the display device. The advantage of the embodiment is that the display device can be communicated with, without having to have electric wires.

According to one embodiment, the first communication unit is configured to communicate wirelessly with the display device using a first communication frequency, the second communication unit is configured to communicate wirelessly with the implantable medical device using a second communication frequency, and the first and second communication frequencies are different. The advantage of the embodiment is that the likelihood of interferences is reduced.

According to one embodiment, the second communication unit is configured to communicate wirelessly with the implantable medical device using electromagnetic waves at a frequency below 100 kHz.

According to one embodiment, the second communication unit is configured to communicate wirelessly with the implantable medical device using electromagnetic waves at a frequency below 40 kHz. The advantage of the embodiment is that titanium which is commonly used for medical devices is transparent for electromagnetic waves below 40 kHz.

According to one embodiment, the first communication unit is configured to communicate wirelessly with the display device using electromagnetic waves at a frequency above 100 kHz. The advantage of the embodiment is that the frequency spectrum below 100 kHz remains noise free for the communication with the medical implantable device.

According to one embodiment, the first communication unit is configured to communicate wirelessly with the display device using a first communication protocol, the second communication unit is configured to communicate wirelessly with the implantable medical device using a second communication protocol, and the first and second communication protocols are different. The advantage of the embodiment is that the protocol can be independently chosen for the communication of the first and second communication unit, depending on which protocol better suits the needs of the communication units.

According to one embodiment, the housing unit comprises, a first antenna configured for wireless communication with the display device, and a second antenna configured for wireless communication with the implantable medical device. The advantage of the embodiment is that the antenna can be independently chosen for the communication of the first and second communication unit, depending on which antenna better suits the needs of the communication units.

According to one embodiment, the first communication unit is a wire-based communication unit for wire-based communication with the display device. The advantage of the embodiment is that the communication of the first communication unit is reliable and secure.

According to one embodiment, the display device comprises, a first communication unit for communication with the housing unit, and a second communication unit for wireless communication with a second external device. The advantage of the embodiment is that an additional external device can be communicated with, thereby introducing redundancy and reliability.

According to one embodiment, the second communication unit of the display device is configured for communicating with the second external device over the Internet. The advantage of the embodiment is that the display device can communicate with devices far away.

According to one embodiment, the first communication unit of the display device is a wireless communication unit for wireless communication with the housing unit. The advantage of the embodiment is that the communication unit can be connected to the housing unit without the use of wires.

According to one embodiment, the first communication unit of the display device is configured to communicate wirelessly with the housing unit using a first communication frequency, the second communication unit of the display device is configured to communicate wirelessly with the second external device using a second communication frequency, and the first and second communication frequencies are different. The advantage of the embodiment is that the likelihood of interferences is reduced and the signal to interference and noise ratio is increased.

According to one embodiment, the first communication unit of the display device is configured to communicate wirelessly with the housing unit using a first communication protocol, the second communication unit of the display device is configured to communicate wirelessly with the second external device using a second communication protocol, and the first and second communication protocols are different. The advantage of the embodiment is that the protocol can be independently chosen for the communication of the first and second communication unit, depending on which protocol better suits the needs of the communication units.

According to one embodiment, the display device comprises, a first antenna configured for wireless communication with the housing, and a second antenna configured for wireless communication with the second external device. The advantage of the embodiment is that the antenna can be independently chosen for the communication of the first and second communication unit, depending on which antenna better suits the needs of the communication units.

According to one embodiment, the first communication unit is a wire-based communication unit for wire-based communication with the housing unit. The advantage of the embodiment is that the communication of the first communication unit is reliable and secure.

According to one embodiment, the display device is configured to display a user interface to the patient. The advantage of the embodiment is that the patient can use his familiar display device to communicate with the housing unit.

According to one embodiment, the housing unit is configured to transmit information pertaining to the display of the user interface to the display device. The advantage of the embodiment is that the patient can receive information using his familiar display device.

According to one embodiment, the display device is configured to, receive input pertaining to communication to or from the implantable medical device from the patient, and transmit signals based on the received input to the housing unit. The advantage of the embodiment is that the patient can use his familiar display device to communicate with the housing unit.

According to one embodiment, the display device comprises a touch screen configured to display the user interface and receive the input from the patient. The advantage of the embodiment is that the patient can use a familiar way of interacting with the information.

According to one embodiment, the housing unit is configured to display a user interface to the patient. The advantage of the embodiment is that the housing unit can receive user input.

According to one embodiment, the first communication unit of the housing unit is configured to receive communication from the implantable medical device pertaining to input from the patient, and wirelessly transmit signals based on the received input to the implantable medical device, using the second communication unit. The advantage of the embodiment is that the housing unit acts as an extra node in the communication between the display device and the medical implantable device, allowing it to monitor the communication.

According to one embodiment, the second communication unit of the housing unit is configured for wireless communication with the implantable medical device using a standard network protocol. The advantage of the embodiment is that the implementation of the communication units is cheap and the protocols reliable.

According to one embodiment, the standard network protocol is one from the list of, Radio Frequency type protocol, RFID type protocol, WLAN type protocol, Bluetooth type protocol, BLE type protocol, NFC type protocol, 3G/4G/5G type protocol, and GSM type protocol.

According to one embodiment, the second communication unit of the housing unit comprises a Bluetooth transceiver.

According to one embodiment, the second communication unit of the housing unit is configured for wireless communication with the implantable medical device using a proprietary network protocol.

The advantage of the embodiment is that the housing unit is compatible with implantable medical devices that use proprietary network protocols.

According to one embodiment, the second communication unit of the housing unit comprises a UWB transceiver. The advantage is that high data rates can be communicated via the second communication unit.

According to one embodiment, the first communication unit of the housing unit is configured for wireless communication with the display device using a standard network protocol. The advantage of the embodiment is that the implementation of the communication units is cheap and the protocols reliable.

According to one embodiment, the standard network protocol is an NFC type protocol. The advantage of the embodiment is that the distance between the communicating devices is limited, protecting against eavesdropping attacks.

According to one embodiment, the first communication unit of the housing unit is configured for wireless communication with the display device using a proprietary network protocol. The advantage of the embodiment is that the housing unit is compatible with implantable medical devices that use proprietary network protocols.

According to one embodiment, the communication range of the first communication unit of the housing unit is less than a communication range of the second communication unit of the housing unit. The advantage of the embodiment is that energy is saved by selecting the first communication unit when its range suffices.

According to one embodiment, a communication range of the first communication unit of the display device is less than a communication range of the second communication unit of the display device. The advantage of the embodiment is that energy is saved by selecting the first communication unit when it's range suffices.

According to one embodiment, at least one of the housing unit and the display device is configured to allow communication between the housing unit and the display device on the basis of a distance between the housing unit and the display device. The advantage of the embodiment is that the distance is used as a safety and authorization factor.

According to one embodiment, at least one of the housing unit and the display device is configured to allow communication between the housing unit and the display device on the basis of the housing unit being mechanically connected to the display device. The advantage of the embodiment is that the safety against a man in the middle attacks is increased.

According to one embodiment, the housing unit is configured to allow communication between the housing unit and the implantable medical device on the basis of a distance between the housing unit and the implantable medical device. The advantage of the embodiment is that the distance is used as a safety and authorization factor.

According to one embodiment, the housing unit further comprises an encryption unit configured to encrypt communication received from the display device. The advantage of the embodiment is that the encrypted communication is protected against unwanted third-party access.

According to one embodiment, the housing unit is further adapted to transmit the encrypted communication, using the second communication unit, to the implantable medical device. The advantage of the embodiment is that the encrypted communication is protected against unwanted third-party access.

According to one embodiment, the second communication unit of the display device is configured to be disabled to enable at least one of, communication between the display device and the housing unit, and communication between the housing unit and the implantable medical device.

The display device in any of the embodiment herein could be a wearable device or a handset. The advantage of the embodiment is that the device is mobile and can be used where needed.

According to one embodiment, the housing unit comprises a case for the wearable device or handset. The advantage of the embodiment is that the wearable device or handset can be protected from mechanical trauma.

According to one embodiment, the implantable medical device is an implantable medical device configured to exert a force on a body portion of the patient and the implantable medical device may comprise an electrical motor and a controller for controlling the electrical motor. The advantage of the embodiment is that the motor and the motor controller enables manipulation of the patient's body in a controlled fashion.

The implantable medical device may comprise at least one of, an external heart compression device, an apparatus assisting the pump function of a heart of the patient, an apparatus assisting the pump function comprising a turbine bump placed within a patient's blood vessel for assisting the pump function of the heart, an operable artificial heart valve, an operable artificial heart valve for increasing the blood flow to the coronary arteries, an implantable drug delivery device, an implantable drug delivery device for injecting directly into a blood vessel and change the position of the injection site, all from within the patient's body, an implantable drug delivery device for injecting potency enhancing drugs into an erectile tissue of the patient, a hydraulic, mechanic, and/or electric constriction implant, an operable volume filling device, an operable gastric band, an operable implant for stretching the stomach wall of the patient for creating satiety, an implant configured to sense the frequency of the patient ingesting food, an operable cosmetic implant, an operable cosmetic implant for adjust the shape and/or size in the breast region of a patient, an implant controlling medical device for the emptying of a urinary bladder, an implant hindering urinary leakage, an implant hindering anal incontinence, an implant controlling the emptying of fecal matter, an implant monitoring an aneurysm, an implant for hindering the expansion of an aneurysm, an implant lubricating a joint, an implant for affecting the blood flow to an erectile tissue of the patient, an implant for simulating the engorgement of an erectile tissue, an implant with a reservoir for holding bodily fluids, an implant storing and/or emptying a bodily reservoir or a surgically created reservoir, an implant communicating with a database outside the body, an implant able to be programmed from outside the body, an implant able to be programmed from outside the body with a wireless signal, an implant treating impotence, an implant controlling the flow of eggs in the uterine tube, an implant controlling the flow of sperms in the uterine tube, an implant controlling the flow of sperms in the vas deferens, an implant for hindering the transportation of the sperm in the vas deferens, an implant treating osteoarthritis, an implant performing a test of parameters inside the body, an implant controlling specific treatment parameters from inside the body, an implant controlling bodily parameters from inside the body, an implant controlling the blood pressure, an implant controlling the blood pressure by affecting the dilatation of the renal artery, an implant controlling a drug treatment parameter, an implant controlling a parameter in the blood, an implant for adjusting or replacing any bone part of a body of the patient, an implant replacing an organ of the patient or part of an organ of the patient or the function thereof, a vascular treatment device, an implant adapted to move fluid inside the body of the patient, an implant configured to sense a parameter related to the patient swallowing, an implant configured to exercise a muscle with electrical or mechanical stimulation, an implant configured for emptying an intestine portion on command, an operable implant configured to be invaginated in the stomach of the patient to reduce the volume of the stomach substantially more than the volume of the device, an implant configured for emptying the urinary bladder from within the patient's body by compressing the bladder, an implant configured for draining fluid from within the patient's body, an implant configured for the active lubrication of a joint with an added lubrication fluid, an implant configured for removing clots and particles from the patient's blood stream, an implant configured for elongating or straightening a bone in the patient, to reduce scoliosis, a device to stimulate the brain for a several position to a focused point, an artificial stomach replacing the function of the natural stomach, an implant configured for adjusting the position of a female's urinary tract or bladder neck, an implant configured for stimulating the ampulla vas deference and creating temporary constriction.

A housing unit configured for communication with an implantable medical device when implanted in a patient is further provided. The housing unit being configured to mechanically connect to a display device and comprising, a first communication unit for communication with the display device, a second communication unit for wireless communication with the implantable medical device.

According to one embodiment, the display device is a wearable device or a handset and the housing unit comprises a case for the wearable device or handset.

According to one embodiment, the first communication unit is a wireless communication unit for wireless communication with the display device.

According to one embodiment, the first communication unit is configured to communicate wirelessly with the display device using a first communication frequency, the second communication unit is configured to communicate wirelessly with the implantable medical device using a second communication frequency, and the first and second communication frequencies are different.

According to one embodiment, the housing unit is configured to transmit information pertaining to the display of a user interface to the display device.

According to one embodiment, the housing unit is configured to receive patient input from the display device.

According to one embodiment, the housing unit is configured to display a user interface to the patient.

According to one embodiment, the housing unit is configured allow communication between the housing unit and the display device on the basis of a distance between the housing unit and the display device.

According to one embodiment, the housing unit is configured allow communication between the housing unit and the display device on the basis of the housing unit being mechanically connected to the display device.

According to one embodiment, the housing unit is configured allow communication between the housing unit and the implantable medical device on the basis of a distance between the housing unit and the implantable medical device.

According to one embodiment, the housing unit further comprises an encryption unit configured to encrypt communication received from the display device.

According to one embodiment, the housing unit is further adapted to transmit the encrypted communication, using the second communication unit, to the implantable medical device.

According to one embodiment, the minimum bounding box of the housing unit and the display device when mechanically connected, is no more than, 10% wider, 10% longer or 100% higher, than the minimum bounding box of the display device.

According to one embodiment, the housing unit comprises one or more switches configured to, when the housing is not mechanically connected to the display device, be used by the patient.

According to one embodiment, the switches are at least partly covered by the display device, when the display device is mechanically connected to the housing unit.

According to one embodiment, at least a part of the housing bends to mechanically connect to the display device.

According to one embodiment, at least a part of the housing is configured to clasp the display device.

According to one embodiment, the housing is configured to cover at least one side of the display device, when mechanically connected to the display device.

According to one embodiment, the housing is configured to be mechanically connected to the display device by a device mechanically connected to the housing and the display device.

### Aspect 331SE eHealth General Security Module

An implantable controller for an implantable medical device is further provided. The implantable controller comprises a wireless transceiver for communicating wirelessly with an external device, a security module, and a central unit configured to be in communication with the wireless transceiver, the security module and the implantable medical device. The wireless transceiver is configured to receive communication from the external device including at least one instruction to the implantable medical device and transmit the received communication to the central unit. The central unit is configured to send secure communication to the security module, derived from the received communication from the external device, and the security module is configured to decrypt at least a portion of the secure communication and/or verify the authenticity of the secure communication. The security module is configured to transmit a response communication to the central unit, and the central unit is configured to communicate the at least one instruction to the implantable medical device, the at least one instruction being based on the response communication, or a combination of the response communication and the received communication from the external device.

According to one embodiment, the security module comprises a set of rules for accepting communication from the central unit.

According to one embodiment, the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.

According to one embodiment, the set of rules comprises a rule stipulating that communication from the central unit is only accepted when the wireless transceiver has been placed in the off-mode for a specific time period.

According to one embodiment, the central unit is configured to verify a digital signature of the received communication from the external device.

According to one embodiment, the set of rules comprises a rule stipulating that communication from the central unit is only accepted when the digital signature of the received communication has been verified by the central unit.

According to one embodiment, the central unit is configured to verify the size of the received communication from the external device.

According to one embodiment, the set of rules comprises a rule stipulating that communication from the central unit is only accepted when the size of the received communication has been verified by the central unit.

The wireless transceiver of any of the preceding embodiments may be configured to receive a message from the external device being encrypted with at least a first and second layer of encryption and the central unit may be configured to decrypt a first layer of decryption and transmit at least a portion of the message comprising the second layer of encryption to the security model. The security module may be configured to decrypt the second layer of encryption and transmit a response communication to the central unit based on the portion of the message decrypted by the security module.

According to one embodiment, the central unit may be configured to decrypt a portion of the message comprising a digital signature, such that the digital signature can be verified by the central unit.

According to one embodiment, the central unit is configured to decrypt a portion of the message comprising message size information, such that the message size can be verified by the central unit.

According to one embodiment, the central unit is configured to decrypt a first and second portion of the message, and the first portion comprises a checksum for verifying the authenticity of the second portion.

According to one embodiment, the response communication transmitted from the security module comprises a checksum, and the central unit may be configured to verify the authenticity of at least a portion of the message decrypted by the central unit using the received checksum.

According to one embodiment, the set of rules comprises a rule related to the rate of data transfer between the central unit and the security module.

The security module in any of the embodiments herein may be configured to decrypt a portion of the message comprising a digital signature, encrypted with the second layer of encryption, such that the digital signature can be verified by the security module.

The central unit could be configured such that it is only capable of decrypting a portion of the receive communication from the external device when the wireless transceiver is placed in the off-mode.

According to one embodiment, the central unit is only capable of communicating the at least one instruction to the implantable medical device when the wireless transceiver is placed in the off-mode.

According to one embodiment, the implantable controller is configured to receive, using the wireless transceiver, a message from the external device comprising a first un-encrypted portion and a second encrypted portion, decrypt the encrypted portion, and use the decrypted portion to verify the authenticity of the un-encrypted portion.

According to one embodiment, the central unit is configured to transmit the encrypted portion to the security module, receive a response communication from the security module, based on information contained in the encrypted portion being decrypted by the security module, and use the response communication to verify the authenticity of the un-encrypted portion.

According to one embodiment, the un-encrypted portion comprises at least a portion of the at least one instruction to the implantable medical device.

The implantable controller may be configured to receive, using the wireless transceiver, a message from the external device comprising information related to at least one of: a physiological parameter of the patient and a physical parameter of the implanted medical device, and use the received information to verify the authenticity of the message.

The physiological parameter of the patient may comprise at least one of: a temperature, a heart rate and a saturation value.

The physical or functional parameter of the implanted medical device may comprise at least one of: a current setting or value of the implanted medical device, a prior instruction sent to the implanted medical device or an ID of the implanted medical device.

According to one embodiment, the portion of the message comprising the information is encrypted, and the central unit is configured to transmit the encrypted portion to the security module and receive a response communication from the security module, based on the information having been decrypted by the security module.

According to one embodiment, the security module comprises a hardware security module comprising at least one hardware-based key. The hardware-based key may correspond to a hardware-based key in the external device, which may be a hardware-based key on a key-card connectable to the external device.

According to one embodiment, the security module comprises a software security module comprising at least one software-based key. The software-based key may correspond to a software-based key in the external device. The software-based key could correspond to a software-based key on a key-card connectable to the external device. The security module may in any of the embodiments comprise a combination of a software-based key and a hardware-based key.

The implantable controller may in any of the preceding embodiments comprise at least one crypto processor.

The wireless transceiver may in any of the embodiments be configured to receive communication from a handheld external device.

According to one embodiment, the at least one instruction to the implantable medical device may comprise an instruction for changing an operational state of the implantable medical device.

The wireless transceiver may be configured to communicate wirelessly with the external device using electromagnetic waves at a frequency below 100 kHz or at a frequency below 40 kHz.

According to one embodiment, the wireless transceiver is configured to communicate wirelessly with the external device using a first communication protocol, and the central unit is configured to communicate with the security module using a second different communication protocol.

The wireless transceiver may in any of the embodiments herein be configured to communicate wirelessly with the external device using a standard network protocol. The standard network protocol may be selected from a list comprising RFID type protocols, WLAN type protocols, Bluetooth type protocols, BLE type protocols, NFC type protocols, 3G/4G/5G type protocols, and GSM type protocols.

The wireless transceiver may in some embodiments be configured to communicate wirelessly with the external device using a proprietary network protocol.

According to one embodiment, the wireless transceiver comprises a UWB transceiver.

According to one embodiment, the security module and/or the central unit and/or the wireless transceiver are comprised in the controller.

The external unit in any of the embodiments herein could be a wearable device or a handset. The advantage of the embodiment is that the device is mobile and can be used where needed.

According to one embodiment, the implantable medical device is an implantable medical device configured to exert a force on a body portion of the patient and the implantable medical device may comprise an electrical motor and a controller for controlling the electrical motor. The advantage of the embodiment is that the motor and the motor controller enables manipulation of the patient's body in a controlled fashion.

The implantable medical device may comprise at least one of, an external heart compression device, an apparatus assisting the pump function of a heart of the patient, an apparatus assisting the pump function comprising a turbine bump placed within a patient's blood vessel for assisting the pump function of the heart, an operable artificial heart valve, an operable artificial heart valve for increasing the blood flow to the coronary arteries, an implantable drug delivery device, an implantable drug delivery device for injecting directly into a blood vessel and change the position of the injection site, all from within the patient's body, an implantable drug delivery device for injecting potency enhancing drugs into an erectile tissue of the patient, a hydraulic, mechanic, and/or electric constriction implant, an operable volume filling device, an operable gastric band, an operable implant for stretching the stomach wall of the patient for creating satiety, an implant configured to sense the frequency of the patient ingesting food, an operable cosmetic implant, an operable cosmetic implant for adjust the shape and/or size in the breast region of a patient, an implant controlling medical device for the emptying of a urinary bladder, an implant hindering urinary leakage, an implant hindering anal incontinence, an implant controlling the emptying of fecal matter, an implant monitoring an aneurysm, an implant for hindering the expansion of an aneurysm, an implant lubricating a joint, an implant for affecting the blood flow to an erectile tissue of the patient, an implant for simulating the engorgement of an erectile tissue, an implant with a reservoir for holding bodily fluids, an implant storing and/or emptying a bodily reservoir or a surgically created reservoir, an implant communicating with a database outside the body, an implant able to be programmed from outside the body, an implant able to be programmed from outside the body with a wireless signal, an implant treating impotence, an implant controlling the flow of eggs in the uterine tube, an implant controlling the flow of sperms in the uterine tube, an implant controlling the flow of sperms in the vas deferens, an implant for hindering the transportation of the sperm in the vas deferens, an implant treating osteoarthritis, an implant performing a test of parameters inside the body, an implant controlling specific treatment parameters from inside the body, an implant controlling bodily parameters from inside the body, an implant controlling the blood pressure, an implant controlling the blood pressure by affecting the dilatation of the renal artery, an implant controlling a drug treatment parameter, an implant controlling a parameter in the blood, an implant for adjusting or replacing any bone part of a body of the patient, an implant replacing an organ of the patient or part of an organ of the patient or the function thereof, a vascular treatment device, an implant adapted to move fluid inside the body of the patient, an implant configured to sense a parameter related to the patient swallowing, an implant configured to exercise a muscle with electrical or mechanical stimulation, an implant configured for emptying an intestine portion on command, an operable implant configured to be invaginated in the stomach of the patient to reduce the volume of the stomach substantially more than the volume of the device, an implant configured for emptying the urinary bladder from within the patient's body by compressing the bladder, an implant configured for draining fluid from within the patient's body, an implant configured for the active lubrication of a joint with an added lubrication fluid, an implant configured for removing clots and particles from the patient's blood stream, an implant configured for elongating or straightening a bone in the patient, to reduce scoliosis, a device to stimulate the brain for a several position to a focused point, an artificial stomach replacing the function of the natural stomach, an implant configured for adjusting the position of a female's urinary tract or bladder neck, an implant configured for stimulating the ampulla vas deference and creating temporary constriction.

An implantable medical device comprising a receiving unit is further provided. The implantable medical device comprises at least one coil configured for receiving transcutaneously transferred energy, a measurement unit configured to measure a parameter related to the energy received by the coil, a variable impedance electrically connected to the coil, a switch placed between the variable impedance and the coil for switching off the electrical connection between the variable impedance and the coil. The implantable medical device further comprises a controller configured to control at least one of the variable impedance for varying the impedance and thereby tune the coil based on the measured parameter, and the switch for switching off the electrical connection between the variable impedance and the coil in response to the measured parameter exceeding a threshold value.

According to one embodiment, the controller is configured to vary the variable impedance in response to the measured parameter exceeding a threshold value.

According to one embodiment, the measurement unit is configured to measure a parameter related to the energy received by the coil over a time period.

According to one embodiment, the measurement unit is configured to measure a parameter related to a change in energy received by the coil.

According to one embodiment, the first switch is placed at a first end portion of the coil, and the implantable medical device further comprises a second switch placed at a second end portion of the coil, such that the coil can be completely disconnected from other portions of the implantable medical device.

According to one embodiment, the receiving unit is configured to receive transcutaneously transferred energy in pulses according to a pulse pattern, and the measurement unit is configured to measure a parameter related to the pulse pattern.

According to one embodiment, the controller is configured to control the variable impedance in response to the pulse pattern deviating from a predefined pulse pattern.

According to one embodiment, the controller is configured to control the switch for switching off the electrical connection between the variable impedance and the coil in response to the pulse pattern deviating from a predefined pulse pattern.

According to one embodiment, the measurement unit is configured to measure a temperature in the implantable medical device or in the body of the patient, and the controller is configured to control the first and second switch in response to the measured temperature.

According to one embodiment, the variable impedance comprises a resistor and a capacitor, a resistor and an inductor and/or an inductor and a capacitor.

The variable impedance may comprise a digitally tuned capacitor. The variable impedance may comprise a digital potentiometer. The variable impedance may comprise a variable inductor.

According to one embodiment, the variation of the impedance is configured to lower the active power that is received by the receiving unit.

According to one embodiment, the variable impedance is placed in series with the coil.

According to one embodiment, the variable impedance is placed parallel to the coil.

According to one embodiment, the implantable medical device further comprises an energy storage unit connected to the receiving unit. The energy storage unit is configured for storing energy received by the receiving unit.

The implantable medical device according to any one of the preceding embodiments may further comprise an energy consuming part. The energy consuming part of the implantable medical device may be configured to exert a force on a body portion of the patient. According to one embodiment, the energy consuming part of the implantable medical device comprises an electrical motor, and the controller is configured for controlling the electrical motor.

According to one embodiment, the energy consuming part comprises at least one of an external heart compression device, an apparatus assisting the pump function of a heart of the patient, an apparatus assisting the pump function comprising a turbine bump placed within a patient's blood vessel for assisting the pump function of the heart, an operable artificial heart valve, an operable artificial heart valve for increasing the blood flow to the coronary arteries, an implantable drug delivery device, an implantable drug delivery device for injecting directly into a blood vessel and change the position of the injection site, all from within the patient's body, an implantable drug delivery device for injecting potency enhancing drugs into an erectile tissue of the patient, a hydraulic, mechanic, and/or electric constriction implant, an operable volume filling device, an operable gastric band, an operable implant for stretching the stomach wall of the patient for creating satiety, an implant configured to sense the frequency of the patient ingesting food, an operable cosmetic implant, an operable cosmetic implant for adjust the shape and/or size in the breast region of a patient, an implant controlling medical device for the emptying of a urinary bladder, an implant hindering urinary leakage, an implant hindering anal incontinence, an implant controlling the emptying of fecal matter, an implant monitoring an aneurysm, an implant for hindering the expansion of an aneurysm, an implant lubricating a joint, an implant for affecting the blood flow to an erectile tissue of the patient, an implant for simulating the engorgement of an erectile tissue, an implant with a reservoir for holding bodily fluids, an implant storing and/or emptying a bodily reservoir or a surgically created reservoir, an implant communicating with a database outside the body, an implant able to be programmed from outside the body, an implant able to be programmed from outside the body with a wireless signal, an implant treating impotence, an implant controlling the flow of eggs in the uterine tube, an implant controlling the flow of sperms in the uterine tube, an implant controlling the flow of sperms in the vas deferens, an implant for hindering the transportation of the sperm in the vas deferens, an implant treating osteoarthritis, an implant performing a test of parameters inside the body, an implant controlling specific treatment parameters from inside the body, an implant controlling bodily parameters from inside the body, an implant controlling the blood pressure, an implant controlling the blood pressure by affecting the dilatation of the renal artery, an implant controlling a drug treatment parameter, an implant controlling a parameter in the blood, an implant for adjusting or replacing any bone part of a body of the patient, an implant replacing an organ of the patient or part of an organ of the patient or the function thereof, a vascular treatment device, an implant adapted to move fluid inside the body of the patient, an implant configured to sense a parameter related to the patient swallowing, an implant configured to exercise a muscle with electrical or mechanical stimulation, an implant configured for emptying an intestine portion on command, an operable implant configured to be invaginated in the stomach of the patient to reduce the volume of the stomach substantially more than the volume of the device, an implant configured for emptying the urinary bladder from within the patient's body by compressing the bladder, an implant configured for draining fluid from within the patient's body, an implant configured for the active lubrication of a joint with an added lubrication fluid, an implant configured for removing clots and particles from the patient's blood stream, an implant configured for elongating or straightening a bone in the patient, to reduce scoliosis, a device to stimulate the brain for a several position to a focused point, an artificial stomach replacing the function of the natural stomach, an implant configured for adjusting the position of a female's urinary tract or bladder neck, an implant configured for stimulating the ampulla vas deference and creating temporary constriction.

### A: Pop-Rivet—Fixation and Housing of Control Units/Controllers and Operation Devices

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Embodiments of implantable devices for fixation and housing of implantable control units/controllers for controlling and communicating with at least one powered/operable implant, as well as for fixation and housing of operation devices for operating elements of powered/operable implantable medical device, will be described in the following. Such implantable devices for fixation and housing may be referred to as remote units or implantable energized medical devices.

According to one embodiment of the inventive concept, these and other objects are achieved in full, or at least in part, by an implantable energized medical device configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion, wherein the connecting portion is configured to connect the first portion to the second portion, wherein: the first, second, third and fourth planes are parallel to each other, the third cross-sectional area is smaller than the second and fourth cross-sectional areas, such that the first portion, second portion and connecting portion are prevented from travelling through the hole in the tissue portion in a direction perpendicular to the first, second and third planes, and the first portion is detachably connected to at least one of the connecting portion and the second portion.

In some embodiments, the third cross-sectional area is smaller than the first cross-sectional area.

In some embodiments, the third cross-sectional area is equal to or larger than the first cross-sectional area.

In some embodiments, the connecting portion comprises a flange comprising the fourth cross-sectional area, such that the flange is prevented from travelling through the hole in the tissue portion in a direction perpendicular to the first, second and third planes.

In some embodiments, the flange protrudes in a direction parallel to the first, second, third and fourth planes, and perpendicular to a central extension of the connecting portion.

In some embodiments, the flange comprises the third surface configured to engage the first tissue surface of the first side of the tissue portion.

In some embodiments, the connecting portion comprises at least one protruding element comprising the fourth cross-sectional area, such that the at least one protruding element is prevented from travelling through the hole in the tissue portion, such that the second portion and the connecting portion can be held in position by the tissue portion of the patient also when the first portion is disconnected from the connecting portion.

In some embodiments, the at least one protruding element protrudes in a direction parallel to the first, second, third and fourth planes, and perpendicular to a central extension of the connecting portion.

In some embodiments, the at least one protruding element comprises the third surface configured to engage the first tissue surface of the first side of the tissue portion.

In some embodiments, the connecting portion comprises at least two protruding elements comprising the fourth cross-sectional area.

In some embodiments, the at least two protruding elements are symmetrically arranged about a central axis of the connecting portion.

In some embodiments, the at least two protruding elements are asymmetrically arranged about a central axis of the connecting portion.

In some embodiments, at least one of the first, second and third surfaces comprises at least one of ribs, barbs, hooks, a friction enhancing surface treatment, and a friction enhancing material, to facilitate the implantable energized medical device being held in position by the tissue portion.

In some embodiments, the connecting portion comprises a hollow portion.

In some embodiments, the hollow portion provides a passage between the first and second portions.

In some embodiments, the first portion is detachably connected to the connecting portion by at least one of a mechanical connection and a magnetic connection.

In some embodiments, the first portion is detachably connected to the connecting portion by at least one of threads and corresponding grooves, a screw, a self-locking element, a twist and lock fitting, and a spring-loaded locking mechanism.

In some embodiments, the at least one protruding element has a height in a direction perpendicular to the fourth plane being less than a height of the first portion in said direction.

In some embodiments, the at least one protruding element has a height in said direction perpendicular to the fourth plane being less than half of said height of the first portion in said direction.

In some embodiments, the at least one protruding element has a height in said direction perpendicular to the fourth plane being less than a quarter of said height of the first portion in said direction.

In some embodiments, the at least one protruding element has a height in said direction perpendicular to the fourth plane being less than a tenth of said height of the first portion in said direction.

In some embodiments, the at least one protruding element has a diameter in the fourth plane being one of: less than a diameter of the first portion in the first plane, equal to a diameter of the first portion in the first plane, and larger than a diameter of the first portion in the first plane.

In some embodiments, the at least one protruding element has a cross-sectional area in the fourth plane being one of: less than a cross-sectional area of the first portion in the first plane, equal to a cross-sectional area of the first portion in the first plane, and larger than a cross-sectional area of the first portion in the first plane.

In some embodiments, the at least one protruding element has a height in said direction perpendicular to the fourth plane being less than half of a height of the connecting portion in said direction.

In some embodiments, the at least one protruding element has a height in said direction perpendicular to the fourth plane being less than a quarter of said height of the connecting portion in said direction.

In some embodiments, the at least one protruding element has a height in said direction perpendicular to the fourth plane being less than a tenth of said height of the connecting portion in said direction.

According to one embodiment of the inventive concept, it is provided an implantable energized medical device configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, and a connecting interface between the connecting portion and the second portion is excentric with respect to the second portion.

In some embodiments, the connecting interface between the connecting portion and the second portion is excentric, with respect to the second portion, in a first direction, but not in a second direction being perpendicular to the first direction.

In some embodiments, the connecting interface between the connecting portion and the second portion is excentric, with respect to the second portion, in a first direction and in a second direction being perpendicular to the first direction.

In some embodiments, the first direction and second direction are parallel to the second plane.

In some embodiments, the second portion has a first end and a second end opposing the first end, wherein the second portion has a length between the first and second end.

In some embodiments, the first end and second end are separated in a direction parallel to the second plane.

In some embodiments, the second portion is curved along the length.

In some embodiments, the second portion is curved in said first direction and said second direction being perpendicular to the first direction.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the second portion has said length in a direction being different to a central extension of the connecting portion.

In some embodiments, the second portion has a proximal region, an intermediate region, and a distal region.

In some embodiments, the proximal region extends from the first end to an interface between the connecting portion and the second portion, the intermediate region is defined by the connecting interface between the connecting portion and the second portion, and the distal region extends from the interface between the connecting portion and the second portion to the second end.

In some embodiments, the proximal region is shorter than the distal region with respect to the length of the second portion.

In some embodiments, the proximal region and the intermediate region together are shorter than the distal region with respect to the length of the second portion.

In some embodiments, the proximal region and the distal region comprises the second surface configured to engage the second surface of the second side of the tissue portion.

In some embodiments, the second portion has a length x and a width y along respective length and width directions being perpendicular to each other and substantially parallel to the second plane, wherein the connecting interface between the connecting portion and the second portion is contained within a region extending from x>0 to x<x/2 and/or y>0 to y<y/2, x and y and 0 being respective end points of the second portion along said length and width directions.

In some embodiments, the second portion is tapered from the first end to the second end.

In some embodiments, the second portion is tapered from each of the first end and second end towards the intermediate region of the second portion.

In some embodiments, the first portion has a maximum dimension being in the range of 10 to 40 mm, such as in the range of 10 to 30 mm, such as in the range of 15 to 25 mm.

In some embodiments, the first portion has a diameter being in the range of 10 to 40 mm, such as in the range of 10 to 30 mm, such as in the range of 15 to 25 mm.

In some embodiments, the connecting portion has a maximum dimension in the third plane in the range of 2 to 20 mm, such as in the range of 2 to 15 mm, such as in the range of 5 to 10 mm.

In some embodiments, the second portion has a maximum dimension being in the range of 30 to 90 mm, such as in the range of 30 to 70 mm, such as in the range of 35 to 60 mm.

In some embodiments, the first portion has one or more of a spherical shape, an ellipsoidal shape, a polyhedral shape, an elongated shape, and a flat disk shape.

In some embodiments, the connecting portion has one of an oval cross-section, an elongated cross-section, and a circular cross-section, in a plane parallel to the third plane.

In some embodiments, the distal region is configured to be directed downwards in a standing patient.

In some embodiments, the first portion comprises a proximal region extending from an first end to an interface between the connecting portion and the first portion, an intermediate region defined by an connecting interface between the connecting portion and the first portion, and a distal region extending from the interface between the connecting portion and the first portion to a second end of the first portion.

In some embodiments, the first portion has a first height, and the second portion has a second height, both heights being in a direction perpendicular to the first and second planes, wherein the first height is smaller than the second height.

In some embodiments, the first height is less than ⅔ of the second height, such as less than ½ of the second height, such as less than ⅓ of the second height.

In some embodiments, the second end of the second portion comprises connections for connecting to an implant being located in a caudal direction from a location of the implantable energized medical device in the patient.

In some embodiments, the first end of the second portion comprises connections for connecting to an implant being located in a cranial direction from a location of the implantable energized medical device in the patient.

According to one embodiment of the inventive concept, it is provided an implantable energized medical device configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion has a third cross-sectional area in a third plane and is configured to connect the first portion to the second portion, wherein: the first, second and third planes are parallel to each other, the third cross-sectional area is smaller than the first and second cross-sectional areas, such that the first portion and second portion are prevented from travelling through the hole in the tissue portion in a direction perpendicular to the first, second and third planes, the first cross-sectional area has a first cross-sectional distance and a second cross-sectional distance, the first and second cross-sectional distances being perpendicular to each other and the first cross-sectional distance being longer than the second cross-sectional distance, the second cross-sectional area has a first cross-sectional distance and a second cross-sectional distance, the first and second cross-sectional distances being perpendicular to each other and the first cross-sectional distance being longer than the second cross-sectional distance, the first cross-sectional distance of the first cross-sectional area and the first cross-sectional distance of the second cross-sectional area are rotationally displaced in relation to each other with an angle exceeding 45° to facilitate insertion of the second portion through the hole in the tissue portion.

In some embodiments, the first cross-sectional distance of the first cross-sectional area and the first cross-sectional distance of the second cross-sectional area are rotationally displaced in relation to each other with an angle exceeding 60° to facilitate insertion of the second portion through the hole in the tissue portion.

In some embodiments, the first cross-sectional distance of the first cross-sectional area and the first cross-sectional distance of the second cross-sectional area are substantially perpendicular to each other to facilitate insertion of the second portion through the hole in the tissue portion.

In some embodiments, the first cross-sectional distance of the first cross-sectional area and the first cross-sectional distance of the second cross-sectional area are rotationally displaced in relation to each other with an angle exceeding 45° and being less than 135°.

In some embodiments, the cross-sectional area of the first portion is elongated.

In some embodiments, the cross-sectional area of the second portion is elongated.

In some embodiments, the connecting portion is connected eccentrically to the second portion.

In some embodiments, the first cross-sectional distance of the second portion is divided into a first, second and third equal length-portions, and wherein the connecting portion is connected to the second portion along the first length-portion of the first cross-sectional distance.

In some embodiments, the first cross-sectional area of the first portion is elongated.

In some embodiments, the second cross-sectional area of the second portion is elongated.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, and the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

According to one embodiment of the inventive concept it is provided an implantable energized medical device configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the implantable energized medical device further comprises at least one sensor for providing input to at least one of the first and second controller.

In some embodiments, the sensor is a sensor configured to sense a physical parameter of the implantable energized medical device.

In some embodiments, the sensor is a sensor configured to sense at least one of: a temperature of the implantable energized medical device or of a body engaging portion, a parameter related to the power consumption of the implantable energized medical device or of a body engaging portion, a parameter related to a status of at least one of the first and second energy storage unit, a parameter related to the wireless transfer of energy from a source external to the body of the patient, and a hydraulic pressure.

In some embodiments, the sensor is a sensor configured to sense a physiological parameter of the patient.

In some embodiments, the sensor is a sensor configured to sense at least one of: a parameter related to the patient swallowing, a local temperature, a systemic temperature, blood saturation, blood oxygenation, blood pressure, a parameter related to an ischemia marker, and pH.

In some embodiments, the sensor configured to sense a parameter related to the patient swallowing comprises at least one of: a motility sensor, a sonic sensor, an optical sensor, and a strain sensor.

In some embodiments, the sensor configured to sense pH is configured to sense the acidity in the stomach.

In some embodiments, the controller is configured to transmit information based on sensor input to a device external to the body of the patient.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the velocity and increase the force of the movement generated by the electrical motor.

In some embodiments, the transmission is configured to transfer a week force with a high velocity into a stronger force with lower velocity.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the implantable energized medical device further comprises a capacitor connected to at least one of the first and second energy storage unit and connected to the electrical motor, wherein the capacitor is configured to: be charged by at least one of the first and second energy storage units, and provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

According to one embodiment of the inventive concept it is provided an implantable energized medical device configured to be held in position by a tissue portion of a patient, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, at least one of the first portion and the second portion comprises at least one coil embedded in a ceramic material, the at least one coil being configured for at least one of receiving energy transmitted wirelessly, transmitting energy wirelessly, receiving wireless communication, and transmitting wireless communication.

In some embodiments, the first portion comprises a first wireless energy receiver configured to receive energy transmitted wirelessly from an external wireless energy transmitter.

In some embodiments, the first portion comprises a first wireless communication receiver.

In some embodiments, the first portion comprises a coil embedded in a ceramic material, hereinafter referred to as a first coil.

In some embodiments, the first wireless energy receiver comprises the first coil.

In some embodiments, the first wireless communication receiver comprises the first coil.

In some embodiments, the first portion comprises a distal end and a proximal end with respect to the connecting portion, along a direction perpendicular to the first plane.

In some embodiments, the first coil is arranged at the distal end of the first portion.

In some embodiments, the first portion comprises an internal wireless energy transmitter.

In some embodiments, the first portion comprises a first wireless communication transmitter.

In some embodiments, the first portion comprises a coil embedded in a ceramic material, hereinafter referred to as a second coil.

In some embodiments, the internal wireless energy transmitter comprises the second coil.

In some embodiments, the first wireless communication transmitter comprises the second coil.

In some embodiments, the second coil is arranged at the proximal end of the first portion.

In some embodiments, the first wireless energy receiver and the internal wireless energy transmitter comprises a single coil embedded in a ceramic material.

In some embodiments, the first wireless communication receiver and the first wireless communication transmitter comprises a single coil embedded in a ceramic material.

In some embodiments, the first wireless energy receiver, the internal wireless energy transmitter, the first wireless communication receiver, and the internal wireless communication transmitter comprises a single coil embedded in a ceramic material.

In some embodiments, the second portion comprises a second wireless energy receiver.

In some embodiments, the second portion comprises a coil embedded in a ceramic material, hereinafter referred to as a third coil, wherein the second wireless energy receiver comprises the third coil.

In some embodiments, the second portion comprises a distal end and a proximal end with respect to the connecting portion, along a direction perpendicular to the first plane.

In some embodiments, the third coil is arranged at the proximal end of the second portion.

In some embodiments, the first portion comprises a first energy storage unit.

In some embodiments, the second portion comprises a second energy storage unit.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first energy storage unit is configured to store less energy than the second energy storage unit, and configured to be charged faster than the second energy storage unit.

In some embodiments, the first energy storage unit has lower energy density than the second energy storage unit.

In some embodiments, the implantable energized medical device further comprises a housing configured to enclose at least the first portion, and wherein a first portion of the housing is made from titanium and a second portion of the housing is made from a ceramic material.

In some embodiments, the housing made from a ceramic material comprises the at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises the at least one coil embedded in the ceramic material.

In some embodiments, the first, second and third planes are parallel to a major extension plane of the tissue.

In some embodiments, the connecting portion further comprises a fourth cross-sectional area in a fourth plane, wherein the fourth plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the fourth plane is parallel to a major extension plane of the tissue.

In some embodiments, a connecting interface between the connecting portion and the second portion is excentric with respect to the second portion.

According to an embodiment of the inventive concept, an implantable device for exerting a force on a body portion of a patient is provided, wherein the implantable device comprises: an implantable energized medical device and an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting a luminary organ of the patient.

In some embodiments, the implantable hydraulic constriction device comprises an implantable hydraulic constriction device for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

In some embodiments, the implantable hydraulic constriction device comprises an implantable hydraulic constriction device for constricting a blood vessel of the patient.

In some embodiments, the implantable hydraulic constriction device for constricting a blood vessel of the patient is configured to constrict the venous blood flow leading from an erectile tissue for promoting the engorgement of the erectile tissue.

In some embodiments, the implantable hydraulic constriction device comprises an implantable hydraulic constriction device for constricting a vas deference of the patient.

In some embodiments, the implantable element configured to exert a force on a body portion of the patient is an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the implantable element configured to exert a force on a body portion of the patient is an implantable element for actively stretching a stomach wall of the patient to create a feeling of satiety.

According to one embodiment of the inventive concept, it is provided a method of implanting an implantable energized medical device, the method comprising: placing a second portion of an implantable energized medical device between a peritoneum and a layer of muscular tissue of the abdominal wall, placing a first portion of the implantable energized medical device between the skin of the patient and a layer of muscular tissue of the abdominal wall, wherein the first and second portions are configured to be connected by a connecting portion extending through at least one layer of muscular tissue of the abdominal wall, placing a body engaging portion of the implantable energized medical device in connection with a tissue or an organ of the patient which is to be affected by the implantable energized medical device, and placing a transferring member, configured to transfer at least one of energy and force from the second portion to the body engaging portion, at least partially between a peritoneum and a layer of muscular tissue of the abdominal wall, such that at least ⅓ of the length of the transferring member is placed on the outside of the peritoneum.

In some embodiments, the transferring member is configured to transfer mechanical force from the second portion to the body engaging portion.

In some embodiments, the transferring member is configured to transfer hydraulic force from the second portion to the body engaging portion.

In some embodiments, the transferring member is configured to transfer electrical energy force from the second portion to the body engaging portion.

In some embodiments, the transferring member is configured to transfer data between the second portion and the body engaging portion.

In some embodiments, the step of placing the transferring member comprises placing the transferring member at least partially between the peritoneum and the layer of muscular tissue of the abdominal wall, such that at least ½ of the length of the transferring member is placed on the outside of the peritoneum of the patient.

In some embodiments, the step of placing the transferring member comprises placing the transferring member at least partially between the peritoneum and the layer of muscular tissue of the abdominal wall, such that at least ⅔ of the length of the transferring member is placed on the outside of the peritoneum of the patient.

In some embodiments, the step of placing the transferring member comprises placing the transferring member entirely outside of the peritoneum of the patient.

In some embodiments, the step of placing the transferring member comprises placing the transferring member such that it extends from the second portion to an area between the rib cage and the peritoneum of the patient, outside of the peritoneum.

In some embodiments, the step of placing the transferring member comprises placing the transferring member such that it extends from the second portion to an area between the stomach and the thoracic diaphragm of the patient.

In some embodiments, the step of placing the transferring member comprises placing the transferring member such that it extends from the second portion to the stomach of the patient.

In some embodiments, the step of placing the transferring member comprises placing the transferring member such that it extends from the second portion to the esophagus of the patient.

In some embodiments, the step of placing the transferring member comprises placing the transferring member such that it extends from the second portion to the retroperitoneal space.

In some embodiments, the step of placing the transferring member comprises placing the transferring member such that it extends from the second portion to an area of the kidneys.

In some embodiments, the step of placing the transferring member comprises placing the transferring member such that it extends from the second portion to the renal arteries.

In some embodiments, the step of placing the transferring member comprises placing the transferring member such that it extends from the second portion to the subperitoneal space, outside of the peritoneum.

In some embodiments, the step of placing the transferring member comprises placing the transferring member such that it extends from the second portion to the urinary bladder, outside of the peritoneum.

In some embodiments, the step of placing the transferring member comprises placing the transferring member such that it extends from the second portion to the urethra, outside of the peritoneum.

In some embodiments, the step of placing the second portion of the implantable energized medical device between the peritoneum and the layer of muscular tissue of the abdominal wall comprises placing the second portion between a first and second layer of muscular tissue of the abdominal wall.

In some embodiments, the step of placing the second portion comprises placing a second portion comprising an electrical motor.

In some embodiments, the step of placing the second portion comprises placing a second portion comprising a hydraulic pump.

In some embodiments, the step of placing the second portion comprises placing a second portion comprising an energy storage unit.

In some embodiments, the step of placing the second portion comprises placing a second portion comprising a receiver for receiving at least one of: energy and communication, wirelessly.

In some embodiments, the step of placing the first portion comprises placing a first portion comprising a transmitter for transmitting at least one of: energy and communication, wirelessly.

In some embodiments, the step of placing the second portion comprises placing a second portion comprising a controller involved in the control of the powered medical device.

In some embodiments, the second portion is elongated and has a length axis extending substantially in the direction of the elongation of the second portion, and wherein the step of placing the second portion comprises placing the second portion such that the length axis is substantially parallel with the cranial-caudal axis of the patient.

In some embodiments, the second portion is elongated and has a length axis extending substantially in the direction of the elongation of the second portion, and wherein the step of placing the second portion comprises placing the second portion such that the length axis is substantially perpendicular with the cranial-caudal axis of the patient.

In some embodiments, the second portion is elongated and has a length axis extending substantially in the direction of the elongation of the second portion, and wherein the step of placing the second portion comprises entering a hole in a layer of muscular tissue of the stomach wall in the direction of the length axis of the second portion and pivoting or angling the second portion after the hole has been entered.

In some embodiments, the step of placing the first portion of the implantable energized medical device between the skin of the patient and a layer of muscular tissue of the abdominal wall comprises placing the first portion in the subcutaneous tissue.

In some embodiments, the step of placing the first portion of the implantable energized medical device between the skin of the patient and a layer of muscular tissue of the abdominal wall comprises placing the first portion between a first and second layer of muscular tissue of the abdominal wall.

In some embodiments, the step of placing the first portion comprises placing a first portion comprising an energy storage unit.

In some embodiments, the step of placing the first portion comprises placing a first portion comprising a receiver for receiving at least one of energy and communication, wirelessly.

In some embodiments, the step of placing the first portion comprises placing a first portion comprising a transmitter for transmitting at least one of energy and communication, wirelessly.

In some embodiments, the step of placing the first portion comprises placing a first portion comprising a controller involved in the control of the powered medical device.

In some embodiments, the first portion is elongated and has a length axis extending substantially in the direction of the elongation of the first portion, and wherein the step of placing the first portion comprises placing the first portion such that the length axis is substantially parallel with the cranial-caudal axis of the patient.

In some embodiments, the first portion is elongated and has a length axis extending substantially in the direction of the elongation of the first portion, and wherein the step of placing the first portion comprises placing the first portion such that the length axis is substantially perpendicular with the cranial-caudal axis of the patient.

In some embodiments, the first portion is elongated and has a first portion length axis extending substantially in the direction of the elongation of the first portion, and the second portion is elongated and has a second portion length axis extending substantially in the direction of the elongation of the second portion, and wherein the step of placing the first and second portions comprises placing the first and second portions such that the first portion length axis and the second portion length axis are placed at an angle in relation to each other exceeding 30°.

In some embodiments, the step of placing the first and second portions comprises placing the first and second portions such that the first portion length axis and the second portion length axis are placed at an angle in relation to each other exceeding 45°.

In some embodiments, the method further comprises the step of placing the connecting portion through at least one layer of muscular tissue of the abdominal wall.

In some embodiments, the first portion, the second portion and the connecting portion are portions of a single unit.

In some embodiments, the method further comprises the step of connecting the first portion to the connecting portion, in situ.

In some embodiments, the method further comprises the step of connecting the second portion to the connecting portion, in situ.

In some embodiments, the method further comprises the step of connecting the transferring member to the first portion.

In some embodiments, the method further comprises the step of connecting the transferring member to the body engaging portion.

In some embodiments, the body engaging portion comprises a medical device for stretching the stomach wall such that a sensation of satiety is created.

In some embodiments, the body engaging portion comprises a constriction device configured to constrict a luminary organ of a patient.

In some embodiments, the body engaging portion comprises an implantable constriction device.

In some embodiments, the implantable constriction device comprises an implantable constriction device for constricting a luminary organ of the patient.

In some embodiments, the implantable constriction device comprises an implantable constriction device for constricting an intestine of the patient.

In some embodiments, the implantable constriction device comprises an implantable constriction device for constricting a colon or rectum of the patient.

In some embodiments, the implantable constriction device comprises an implantable constriction device for constricting the intestine at a region of a stoma of the patient.

In some embodiments, the implantable constriction device comprises an implantable constriction device for constricting a blood vessel of the patient.

In some embodiments, the implantable constriction device for constricting a blood vessel of the patient is configured to constrict the venous blood flow leading from an erectile tissue for promoting the engorgement of the erectile tissue.

In some embodiments, the implantable constriction device for constricting a blood vessel of the patient is configured to constrict the blood flow in the renal artery to affect the patients systemic blood pressure.

In some embodiments, the implantable constriction device comprises an implantable constriction device for constricting a vas deference of the patient.

In some embodiments, the body engaging portion comprises an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the body engaging comprises an element for electrically stimulating a tissue portion of a patient.

According to one embodiment of the inventive concept, it is provided a kit for assembling an implantable energized medical device configured to be held in position by a tissue portion of a patient, the kit comprising: a group of one or more first portions, a group of one or more second portions, a group of one or more connecting portions, wherein at least one of said groups comprises at least two different types of said respective portions; wherein the medical device is a modular device and, when assembled, comprises a selection, from said groups, of one first portion, one second portion, and one connecting portion, wherein: the first portion is configured to be placed on a first side of the tissue portion, the first portion having a first cross-sectional area in a first plane and comprising a first surface configured to face a first tissue surface of the first side of the tissue portion, the second portion is configured to be placed on a second side of the tissue portion, the second side opposing the first side, the second portion having a second cross-sectional area in a second plane and comprising a second surface configured to engage a second tissue surface of the second side of the tissue portion, and the connecting portion is configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, and the third cross-sectional area is smaller than the first and second cross-sectional areas, such that the first portion and second portion are prevented from travelling through the hole in the tissue portion in a direction perpendicular to the first, second and third planes.

In general, any of the embodiments of the implantable energized medical device disclosed herein may form part of such kit, and any features of such embodiments may be combined to form part of such kit.

In some embodiments, the group of one or more first portions comprises a first portion comprising a first energy storage unit.

In some embodiments, the group of one or more first portions comprises a first portion comprising a first wireless energy receiver unit for receiving energy transmitted wirelessly by an external wireless energy transmitter.

In some embodiments, the first energy storage unit is connected to the first wireless energy receiver, wherein the first wireless energy receiver is configured to receive energy transmitted wirelessly by the external wireless energy transmitter and store the received energy in the first energy storage unit.

In some embodiments, the first wireless energy receiver is configured to be physically connected to a second energy storage unit in the second portion.

In some embodiments, the group of one or more first portions comprises a first portion comprising an internal wireless energy transmitter.

In some embodiments, the group of one or more second portions comprises a second portion comprising a second wireless energy receiver configured to receive energy transmitted wirelessly by the internal wireless energy transmitter.

In some embodiments, the internal wireless energy transmitter is configured to transmit energy wirelessly to the second wireless energy receiver.

In some embodiments, the group of one or more second portions comprises a second portion comprising a second energy storage unit connected to the second wireless energy receiver.

In some embodiments, the second wireless energy receiver is configured to receive energy transmitted wirelessly by the internal wireless energy transmitter and store the received energy in the second energy storage unit.

In some embodiments, the group of one or more first portions comprises a first portion being formed as one integral unit with a connecting portion.

In some embodiments, the group of one or more second portions comprises a second portion being formed as one integral unit with a connecting portion.

In some embodiments, one of the group of one or more first, second or connecting portions comprises a first portion, second portion and connecting portion being formed as one integral unit.

In some embodiments, the group of one or more first portions comprises a first portion having a first height along a direction being perpendicular to the first plane, and a first portion having a second height along said direction being perpendicular to the first plane, wherein the second height is larger than the first height.

In some embodiments, the group of one or more first portions comprises a first portion having a first width and/or length along a direction being parallel to the first plane, and a first portion having a second width and/or length along said direction being parallel to the first plane, wherein the second width and/or length is larger than the first width and/or length.

In some embodiments, the group of one or more second portions comprises a second portion having a first height along a direction being perpendicular to the second plane, and a second portion having a second height along said direction being perpendicular to the second plane, wherein the second height is larger than the first height.

In some embodiments, the group of one or more second portions comprises a second portion having a first width and/or length along a direction being parallel to the second plane, and a second portion having a second width and/or length along said direction being parallel to the second plane, wherein the second width and/or length is larger than the first width and/or length.

In some embodiments, the group of one or more connecting portions comprises a connecting portion having a first height along a direction being perpendicular to the third plane, and a connecting portion having a second height along said direction being perpendicular to the third plane, wherein the second height is larger than the first height.

In some embodiments, the group of one or more connecting portions comprises a connecting portion having a first width and/or length along a direction being parallel to the third plane, and a connecting portion having a second width and/or length along said direction being parallel to the third plane, wherein the second width and/or length is larger than the first width and/or length.

In some embodiments, the group of one or more first portions comprises a first portion comprising an injection port for injecting fluid into the first portion.

In some embodiments, the group of one or more connecting portions comprises a connecting portion comprising a hydraulic fluid conduit for hydraulically connecting the first portion to the second portion.

In some embodiments, the group of one or more first portions comprises a first portion comprising a first controller comprising at least one processing unit.

In some embodiments, the group of one or more second portions comprise a second portion comprising a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the group of first portions comprises a first portion comprising a combined coil, wherein the combined coil is configured to receive wireless energy wirelessly from an external wireless energy transmitter, and transmit wireless energy wirelessly to the second wireless receiver of the second portion.

In some embodiments, at least one of the coils are embedded in a ceramic material.

In some embodiments, the group of one or more first portions comprises a first portion comprising a push button and/or a capacitive button for controlling a function of the implantable energized medical device.

According to one embodiment of the inventive concept, it is provided an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the connecting portion and second portion are configured to form a connecting interface between the connecting portion and the second portion, and the second portion extends along a first direction being parallel to the second plane, wherein the second portion has a lengthwise cross-sectional area along the first direction, wherein a second lengthwise cross-sectional area is smaller than a first lengthwise cross-sectional area and wherein the first lengthwise cross-sectional area is located closer to said connecting interface with regard to the first direction.

In some embodiments, the second portion has a first end and a second end opposing the first end along the first direction, wherein the second portion has a length between the first and second end, and wherein the second portion has an intermediate region and a distal region, wherein the intermediate region is defined by the connecting interface between the connecting portion and the second portion, and the distal region extends from the connecting interface between the connecting portion and the second portion to the second end.

In some embodiments, the lengthwise cross-sectional area of the second portion decreases continuously from an end of the intermediate region towards the second end.

In some embodiments, the lengthwise cross-sectional area of the second portion decreases linearly from an end of the intermediate region towards the second end.

In some embodiments, the lengthwise cross-sectional area of the second portion decreases stepwise from an end of the intermediate region towards the second end.

In some embodiments, the distal region of the second portion is conically shaped.

In some embodiments, the second portion has rotational symmetry along the first direction.

In some embodiments, the second surface of the second portion is substantially perpendicular to a central extension of the connecting portion.

In some embodiments, the second surface of the second portion is substantially parallel to the second plane.

In some embodiments, the second surface of the second portion is substantially flat and configured to form a contact area to the second tissue surface, and wherein the second portion further comprises a lower surface facing away from the first portion configured to taper towards the second end.

In some embodiments, the second portion has a proximal region, wherein the proximal region extends from the first end to the connecting interface between the connecting portion and the second portion.

In some embodiments, the lengthwise cross-sectional area of the second portion decreases continuously from an end of the intermediate region towards the first end.

In some embodiments, the lengthwise cross-sectional area of the second portion decreases linearly from an end of the intermediate region towards the first end.

In some embodiments, the lengthwise cross-sectional area of the second portion decreases stepwise from an end of the intermediate region towards the first end.

In some embodiments, the proximal region of the second portion is conically shaped.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the second portion has said length in a direction being different to a central extension of the connecting portion.

In some embodiments, the connecting interface between the connecting portion and the second portion is excentric with respect to the second portion.

In some embodiments, the connecting interface between the connecting portion and the second portion is excentric, with respect to the second portion, in the first direction, but not in a second direction being perpendicular to the first direction.

In some embodiments, the connecting interface between the connecting portion and the second portion is excentric, with respect to the second portion, in the first direction and in a second direction being perpendicular to the first direction.

In some embodiments, the second direction is parallel to the second plane.

In some embodiments, the proximal region and the distal region comprises the second surface configured to engage the second surface of the second side of the tissue portion.

In some embodiments, the second portion is tapered from the first end to the second end.

In some embodiments, the second portion is tapered from the intermediate region of the second portion to each of the first end and second end.

In some embodiments, the first portion has a maximum dimension being in the range of 10 to 40 mm, such as in the range of 10 to 30 mm, such as in the range of 15 to 25 mm.

In some embodiments, the first portion has a diameter being in the range of 10 to 40 mm, such as in the range of 10 to 30 mm, such as in the range of 15 to 25 mm.

In some embodiments, the connecting portion has a maximum dimension in the third plane in the range of 2 to 20 mm, such as in the range of 2 to 15 mm, such as in the range of 5 to 10 mm.

In some embodiments, the second portion has a maximum dimension being in the range of 30 to 90 mm, such as in the range of 30 to 70 mm, such as in the range of 35 to 60 mm.

In some embodiments, the first portion has one or more of a spherical shape, an ellipsoidal shape, a polyhedral shape, an elongated shape, and a flat disk shape.

In some embodiments, the connecting portion has one of an oval cross-section, an elongated cross-section, and a circular cross-section, in a plane parallel to the third plane.

In some embodiments, the distal region is configured to be directed downwards in a standing patient.

In some embodiments, the first portion has a first height, and the second portion has a second height, both heights being in a direction perpendicular to the first and second planes, wherein the first height is smaller than the second height.

In some embodiments, the first height is less than ⅔ of the second height, such as less than ½ of the second height, such as less than ⅓ of the second height.

In some embodiments, the second end of the second portion comprises connections for connecting to an implant being located in a caudal direction from a location of the implantable energized medical device in the patient.

In some embodiments, the first end of the second portion comprises connections for connecting to an implant being located in a cranial direction from a location of the implantable energized medical device in the patient.

In some embodiments, the connecting portion further comprises a fourth cross-sectional area in a fourth plane, wherein the fourth plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the first surface is configured to engage the first tissue surface of the first side of the tissue portion.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises at least one sensor for providing input to at least one of the first and second controller.

In some embodiments, the sensor is a sensor configured to sense a physical parameter of the implantable energized medical device.

In some embodiments, the sensor is a sensor configured to sense at least one of a temperature of the implantable energized medical device or of a body engaging portion, a parameter related to the power consumption of the implantable energized medical device or of a body engaging portion, a parameter related to a status of at least one of the first and second energy storage unit, a parameter related to the wireless transfer of energy from a source external to the body of the patient, and a hydraulic pressure.

In some embodiments, the sensor is a sensor configured to sense a physiological parameter of the patient.

In some embodiments, the sensor is a sensor configured to sense at least one of: a parameter related to the patient swallowing, a local temperature, a systemic temperature, blood saturation, blood oxygenation, blood pressure, a parameter related to an ischemia marker, and pH.

In some embodiments, the sensor configured to sense a parameter related to the patient swallowing comprises at least one of: a motility sensor, a sonic sensor, an optical sensor, and a strain sensor.

In some embodiments, the sensor configured to sense pH is configured to sense the acidity in the stomach.

In some embodiments, the controller is configured to transmit information based on sensor input to a device external to the body of the patient.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the velocity and increase the force of the movement generated by the electrical motor.

In some embodiments, the transmission is configured to transfer a week force with a high velocity into a stronger force with lower velocity.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the implantable energized medical device further comprises a capacitor connected to at least one of the first and second energy storage unit and connected to the electrical motor, wherein the capacitor is configured to: be charged by at least one of the first and second energy storage units, and provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical device further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

In some embodiments, the fourth plane is parallel to a major extension plane of the tissue.

According to an embodiment of the present inventive concept, an implantable device for exerting a force on a body portion of a patient is provided, the implantable device comprising: an implantable energized medical device and an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting a luminary organ of the patient.

In some embodiments, the implantable hydraulic constriction device comprises an implantable hydraulic constriction device for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

In some embodiments, the implantable hydraulic constriction device comprises an implantable hydraulic constriction device for constricting a blood vessel of the patient.

In some embodiments, the implantable hydraulic constriction device for constricting a blood vessel of the patient is configured to constrict the venous blood flow leading from an erectile tissue for promoting the engorgement of the erectile tissue.

In some embodiments, the implantable hydraulic constriction device comprises an implantable hydraulic constriction device for constricting a vas deference of the patient.

In some embodiments, the implantable element configured to exert a force on a body portion of the patient is an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the implantable element configured to exert a force on a body portion of the patient is an implantable element for actively stretching a stomach wall of the patient to create a feeling of satiety.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the first and second cross-sectional area, such that the first portion, second portion and connecting portion are prevented from travelling through the hole in the tissue portion in a direction perpendicular to the first, second and third planes, and the first portion is configured to be movable in relation to the connecting portion, and/or comprises a first element and a second element, the first element being configured to be moved in relation to the second element to increase an area of the first surface.

In some embodiments, the connecting portion is configured to extend along a central extension between the first portion and the second portion, and wherein the first portion is configured to be moveable to assume several positions along a direction perpendicular to the central extension.

In some embodiments, the first portion is configured to be fixed in the several positions by a locking mechanism arranged on either or both of the first portion and connecting portion.

In some embodiments, the first element is configured to assume a first state, wherein the first element is arranged on top of the second element or within the second element, and a second state, wherein the first element is arranged adjacent to the second element.

In some embodiments, the first element is hingedly connected to the second element.

In some embodiments, the first element and the second element are integrally formed, and wherein the first portion is flexible to allow the first element to fold over the second element to assume the first state.

In some embodiments, the second element comprises a slot, and wherein the first element is configured to be partially or fully housed within the slot in the first state, and wherein the first element is configured to protrude from the slot in the second state.

In some embodiments, the first element comprises a slot, and wherein the second element is configured to be partially or fully housed within the slot in a first state of the second element, and wherein the second element is configured to protrude from the slot in a second state of the second element.

In some embodiments, the first element is configured to rotate about an axis being parallel to said central extension.

In some embodiments, the first element is configured to rotate up to a maximum of 180 degrees about the axis.

In some embodiments, the first element is configured to rotate up to a maximum of 90 degrees about the axis.

In some embodiments, the second element is configured to be connected to the connecting portion.

In some embodiments, the first element is configured to be moved in relation to the second element to protrude or to further protrude beyond an edge of the second element to increase an area of the first surface.

In some embodiments, the second element is movable in relation to the first element to increase an area of the first surface.

In some embodiments, the first element and the second element are configured to be moved from a first state, wherein ends of the first and second elements respectively point in a direction substantially perpendicular to the first plane, to a second state, wherein said ends of the first and second ends point in one or more directions being substantially parallel to the first plane.

In some embodiments, the first element and the second element are configured to assume an upright position extending away from the connecting portion, and to be moved towards a sideways position being substantially perpendicular to the upright position.

In some embodiments, the connecting portion comprises a protruding element and the first portion comprises a slot, wherein the protruding element is configured to slide within the slot along a predetermined path.

In some embodiments, the protruding element is configured to be interlocked within the slot such that the protruding element can only be removed from the slot in a preconfigured position.

In some embodiments, the protruding element is configured to be interlocked within the slot such that the protruding element is permanently enclosed within the slot, or wherein the protruding element is configured to be interlocked within the slot such that the protruding element is permanently enclosed within the slot.

In some embodiments, the connecting portion further comprises a fourth cross-sectional area in a fourth plane, wherein the fourth plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the connecting portion comprises a flange comprising the fourth cross-sectional area, such that the flange is prevented from travelling through the hole in the tissue portion in a direction perpendicular to the first, second and third planes.

In some embodiments, the flange protrudes in a direction parallel to the first, second, third and fourth planes, and perpendicular to a central extension of the connecting portion.

In some embodiments, the flange comprises the third surface configured to engage the first tissue surface of the first side of the tissue portion.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, and the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises at least one sensor for providing input to at least one of the first and second controller.

In some embodiments, the sensor is a sensor configured to sense a physical parameter of the implantable energized medical device.

In some embodiments, the sensor is a sensor configured to sense at least one of: a temperature of the implantable energized medical device or of a body engaging portion, a parameter related to the power consumption of the implantable energized medical device or of a body engaging portion, a parameter related to a status of at least one of the first and second energy storage unit, a parameter related to the wireless transfer of energy from a source external to the body of the patient, and a hydraulic pressure.

In some embodiments, the sensor is a sensor configured to sense a physiological parameter of the patient.

In some embodiments, the sensor is a sensor configured to sense at least one of: a parameter related to the patient swallowing, a local temperature, a systemic temperature, blood saturation, blood oxygenation, blood pressure, a parameter related to an ischemia marker, and pH.

In some embodiments, the sensor configured to sense a parameter related to the patient swallowing comprises at least one of: a motility sensor, a sonic sensor, an optical sensor, and a strain sensor.

In some embodiments, the sensor configured to sense pH is configured to sense the acidity in the stomach.

In some embodiments, the controller is configured to transmit information based on sensor input to a device external to the body of the patient.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the velocity and increase the force of the movement generated by the electrical motor.

In some embodiments, the transmission is configured to transfer a week force with a high velocity into a stronger force with lower velocity.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the implantable energized medical device further comprises a capacitor connected to at least one of the first and second energy storage unit and connected to the electrical motor, wherein the capacitor is configured to:
be charged by at least one of the first and second energy storage units, and
provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical device further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

In some embodiments, the fourth plane is parallel to a major extension plane of the tissue.

In some embodiments, the third cross-sectional area is smaller than the first cross-sectional area.

In some embodiments, the third cross-sectional area is equal to or larger than the first cross-sectional area.

An implantable device for exerting a force on a body portion of a patient is provided, the device comprising: an implantable energized medical device, and an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting a luminary organ of the patient.

In some embodiments, the implantable hydraulic constriction device comprises an implantable hydraulic constriction device for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

In some embodiments, the implantable hydraulic constriction device comprises an implantable hydraulic constriction device for constricting a blood vessel of the patient.

In some embodiments, the implantable hydraulic constriction device for constricting a blood vessel of the patient is configured to constrict the venous blood flow leading from an erectile tissue for promoting the engorgement of the erectile tissue.

In some embodiments, the implantable hydraulic constriction device comprises an implantable hydraulic constriction device for constricting a vas deference of the patient.

In some embodiments, the implantable element configured to exert a force on a body portion of the patient is an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the implantable element configured to exert a force on a body portion of the patient is an implantable element for actively stretching a stomach wall of the patient to create a feeling of satiety.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive electromagnetic waves at a frequency above a frequency level, and/or to transmit electromagnetic waves at a frequency below the frequency level, wherein the second portion is configured to receive and/or transmit electromagnetic waves at a frequency below the frequency level, and wherein the frequency level is 100 kHz.

In some embodiments, wherein the first portion is configured to transmit electromagnetic waves at the frequency below the frequency level to the second portion.

In some embodiments, the first portion is configured to transmit electromagnetic waves at the frequency above the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter above the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device above the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is configured to receive and/or transmit electromagnetic waves at a frequency below the frequency level, and wherein the frequency level is 100 kHz.

In some embodiments, the second portion is configured to receive and/or transmit electromagnetic waves at a frequency below the frequency level.

In some embodiments, the first portion is configured to transmit electromagnetic waves at the frequency below the frequency level to the second portion.

In some embodiments, the first portion is configured to transmit electromagnetic waves at the frequency below the frequency level to an external device.

In some embodiments, the frequency level is 40 kHz or 20 kHz.

In some embodiments, the electromagnetic waves comprise wireless energy and/or wireless communication.

In some embodiments, the first portion comprises a first wireless energy receiver for receiving energy transmitted wirelessly by an external wireless energy transmitter below the frequency level, and an internal wireless energy transmitter configured to transmit energy wirelessly to the second portion below the frequency level, and the second portion comprises a second wireless energy receiver configured to receive energy transmitted wirelessly by the internal wireless energy transmitter below the frequency level.

In some embodiments, the first portion comprises a first controller comprising at least one processing unit.

In some embodiments, the second portion comprises a second controller comprising at least one processing unit.

In some embodiments, the first controller is connected to a first wireless communication receiver in the first portion for receiving wireless communication from an external device below the frequency level, the first controller is connected to a first wireless communication transmitter in the first portion for transmitting wireless communication to a second wireless communication receiver in the second portion below the frequency level.

In some embodiments, the second controller is connected to the second wireless communication receiver for receiving wireless communication from the first portion below the frequency level.

In some embodiments, the first portion comprises an outer casing made from a polymer material.

In some embodiments, the first portion comprises an outer casing made from titanium.

In some embodiments, the outer casing forms a complete enclosure, such that electromagnetic waves received and transmitted by the first portion must travel through the casing.

In some embodiments, the second portion comprises an outer casing made from titanium.

In some embodiments, the outer casing forms a complete enclosure, such that electromagnetic waves received and transmitted by the second portion must travel through the casing.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, the first portion is made from a polymer material, the second portion comprises a casing made from titanium, wherein the casing forms a complete enclosure.

In some embodiments, the casing of the second portion forms a complete enclosure such that the entirety of the outer surface of the second portion is covered by the casing, when the second portion is connected to the connecting portion.

In some embodiments, the first portion comprises a casing made from the polymer material.

In some embodiments, the casing of the first portion forms a complete enclosure such that the entirety of the outer surface of the first portion is covered by the casing.

In some embodiments, the connecting portion comprises a connection arranged to connect to the first and second portion respectively and carry electrical signals and/or energy.

In some embodiments, the connection is arranged in a core of the connecting portion such that it is encapsulated by outer material of the connecting portion.

In some embodiments, the connecting portion comprises a ceramic material.

In some embodiments, the connection is encapsulated within the ceramic material.

In some embodiments, the first portion comprises a first connection configured to connect to the connection of the connecting portion.

In some embodiments, the second portion comprises a second connection configured to connect to the connection of the connection portion.

In some embodiments, the casing of the second portion is hermetically sealed.

In some embodiments, the second connection is arranged such that the hermetical seal of the second portion is kept intact.

In some embodiments, the casing of the first portion is hermetically sealed.

An implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and a third surface configured to engage the first tissue surface of the first side of the tissue portion, wherein the connecting portion is configured to connect the first portion to the second portion, wherein: the first, second, and third planes are parallel to each other, the third cross-sectional area is smaller than the second cross-sectional area, such that the first portion, second portion and connecting portion are prevented from travelling through the hole in the tissue portion in a direction perpendicular to the first, second and third planes, and wherein the connecting portion is configured to extend between the first portion and the second portion along a central extension axis, and wherein the second portion is configured to extend in a length direction being divergent with the central extension axis, and wherein the connecting portion has a substantially constant cross-sectional area along the central extension axis, or wherein the connecting portion has a decreasing cross-sectional area in a direction from the first portion towards the second portion along the central extension axis, and/or wherein the second portion has a substantially constant cross-sectional area along the length direction, or wherein the second portion has a decreasing cross-sectional area in the length direction.

In some embodiments, the third cross-sectional area is smaller than the first cross-sectional area.

In some embodiments, the connecting portion is tapered in the direction from the first portion towards the second portion along the central extension axis.

In some embodiments, the connecting portion has a circular or oval cross-section along the central extension axis with a decreasing diameter in the direction from the first portion towards the second portion.

In some embodiments, the second portion is tapered in the length direction.

In some embodiments, the connecting portion has a circular or oval cross-section in the length direction with a decreasing diameter in the length direction.

In some embodiments, the length direction extends from an interface between the connecting portion and the second portion towards an end of the second portion.

In some embodiments, the length direction extends in a direction substantially perpendicular to the central extension axis.

In some embodiments, the connecting portion comprises a protruding element and the first portion comprises a slot, wherein the protruding element is configured to slide within the slot along a predetermined path.

In some embodiments, the protruding element is configured to be interlocked within the slot such that the protruding element can only be removed from the slot in a preconfigured position.

In some embodiments, the protruding element is configured to be interlocked within the slot such that the protruding element is permanently enclosed within the slot, or wherein the protruding element is configured to be interlocked within the slot such that the protruding element is permanently enclosed within the slot.

In some embodiments, the connecting portion further comprises a fourth cross-sectional area in a fourth plane, wherein the fourth plane is parallel to the first, second and third planes, and wherein the third cross-sectional area is smaller than the fourth cross-sectional area.

In some embodiments, the connecting portion comprises a protruding element comprising the fourth cross-sectional area.

In some embodiments, the connecting portion comprises a flange comprising the fourth cross-sectional area, such that the flange is prevented from travelling through the hole in the tissue portion in a direction perpendicular to the first, second and third planes.

In some embodiments, the flange protrudes in a direction parallel to the first, second, third and fourth planes, and perpendicular to a central extension of the connecting portion.

In some embodiments, the flange comprises the third surface configured to engage the first tissue surface of the first side of the tissue portion.

In some embodiments, the first portion comprises a first wireless energy receiver configured to receive energy transmitted wirelessly from an external wireless energy transmitter.

In some embodiments, the first portion comprises an internal wireless energy transmitter.

In some embodiments, the second portion comprises a second wireless energy receiver.

In some embodiments, the first portion comprises a first energy storage unit.

In some embodiments, the second portion comprises a second energy storage unit.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter, and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the internal wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises at least one sensor for providing input to at least one of the first and second controller.

In some embodiments, the sensor is a sensor configured to sense a physical parameter of the implantable energized medical device.

In some embodiments, the sensor is a sensor configured to sense at least one of: a temperature of the implantable energized medical device or of a body engaging portion, a parameter related to the power consumption of the implantable energized medical device or of a body engaging portion, a parameter related to a status of at least one of the first and second energy storage unit, a parameter related to the wireless transfer of energy from a source external to the body of the patient, and a hydraulic pressure.

In some embodiments, the sensor is a sensor configured to sense a physiological parameter of the patient.

In some embodiments, sensor is a sensor configured to sense at least one of: a parameter related to the patient swallowing, a local temperature, a systemic temperature, blood saturation, blood oxygenation, blood pressure, a parameter related to an ischemia marker, and pH.

In some embodiments, the sensor configured to sense a parameter related to the patient swallowing comprises at least one of: a motility sensor, a sonic sensor, an optical sensor, and a strain sensor.

In some embodiments, the sensor configured to sense pH is configured to sense the acidity in the stomach.

In some embodiments, the controller is configured to transmit information based on sensor input to a device external to the body of the patient.

In some embodiments, the second portion comprises at least a portion of an operation device for operating an implantable body engaging portion.

In some embodiments, the second portion comprises at least one electrical motor.

In some embodiments, the second portion comprises a transmission configured to reduce the velocity and increase the force of the movement generated by the electrical motor.

In some embodiments, the transmission is configured to transfer a week force with a high velocity into a stronger force with lower velocity.

In some embodiments, the transmission is configured to transfer a rotating force into a linear force.

In some embodiments, the transmission comprises a gear system.

In some embodiments, the second portion comprises a magnetic coupling for transferring mechanical work from the electrical motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

In some embodiments, the implantable energized medical device further comprises a capacitor connected to at least one of the first and second energy storage unit and connected to the electrical motor, wherein the capacitor is configured to: be charged by at least one of the first and second energy storage units, and provide the electrical motor with electrical power.

In some embodiments, at least one of the first and second portion comprises a sensation generator adapted to generate a sensation detectable by a sense of the patient.

In some embodiments, the second portion comprises a force transferring element configured to mechanically transfer force from the second portion to an implanted body engaging portion.

In some embodiments, the second portion comprises a force transferring element configured to hydraulically transfer force from the second portion to an implanted body engaging portion.

In some embodiments, the second portion comprises at least one lead for transferring electrical energy and/or information from the second portion to an implanted body engaging portion.

In some embodiments, the first portion comprises an injection port for injecting fluid into the first portion.

In some embodiments, the connecting portion comprises a conduit for transferring a fluid from the first portion to the second portion.

In some embodiments, the conduit is arranged to extend through the hollow portion of the connecting portion.

In some embodiments, the second portion comprises a first and a second chamber separated from each other, wherein the first chamber comprises a first liquid and the second chamber comprises a second liquid, and wherein the second liquid is a hydraulic liquid configured to transfer force to an implantable element configured to exert force on the body portion of the patient.

In some embodiments, a wall portion of the first chamber is resilient to allow an expansion of the first chamber.

In some embodiments, the second portion comprises a first hydraulic system in fluid connection with a first hydraulically operable implantable element configured to exert force on the body portion of the patient, and a second hydraulic system in fluid connection with a second hydraulically operable implantable element configured to exert force on the body portion of the patient, wherein the first and second hydraulically operable implantable elements are adjustable independently from each other.

In some embodiments, the first hydraulic system comprises a first hydraulic pump and the second hydraulic systems comprises a second hydraulic pump.

In some embodiments, each of the first and second hydraulic systems comprises a reservoir for holding hydraulic fluid.

In some embodiments, the implantable energized medical device further comprises a first pressure sensor configured to sense a pressure in the first hydraulic system, and a second pressure sensor configured to sense a pressure in the second hydraulic system.

In some embodiments, the first surface is configured to engage the first tissue surface of the first side of the tissue portion.

In some embodiments, the first, second and third planes are parallel to a major extension plane of the tissue.

In some embodiments, the fourth plane is parallel to a major extension plane of the tissue.

In some embodiments, the third cross-sectional area is smaller than the first cross-sectional area.

In some embodiments, the third cross-sectional area is equal to or larger than the first cross-sectional area.

An implantable device for exerting a force on a body portion of a patient is provided, the device comprising: an implantable energized medical device, an implantable element configured to exert a force on a body portion of the patient.

In some embodiments, the implantable element configured to exert a force on a body portion of the patient is an implantable hydraulic constriction device.

In some embodiments, the implantable hydraulic constriction device is configured for constricting a luminary organ of the patient.

In some embodiments, the implantable hydraulic constriction device comprises an implantable hydraulic constriction device for constricting an intestine of the patient.

In some embodiments, the implantable hydraulic constriction device comprises an implantable hydraulic constriction device for constricting a colon or rectum of the patient.

In some embodiments, the implantable hydraulic constriction device comprises an implantable hydraulic constriction device for constricting the intestine at a region of a stoma of the patient.

In some embodiments, the implantable hydraulic constriction device comprises an implantable hydraulic constriction device for constricting a blood vessel of the patient.

In some embodiments, the implantable hydraulic constriction device for constricting a blood vessel of the patient is configured to constrict the venous blood flow leading from an erectile tissue for promoting the engorgement of the erectile tissue.

In some embodiments, the implantable hydraulic constriction device comprises an implantable hydraulic constriction device for constricting a vas deference of the patient.

In some embodiments, the implantable element configured to exert a force on a body portion of the patient is an implantable element for actively emptying the urinary bladder of the patient.

In some embodiments, the implantable element for actively emptying the urinary bladder of the patient is configured to empty the bladder of the patient by compressing the urinary bladder from the outside thereof.

In some embodiments, the implantable element configured to exert a force on a body portion of the patient is an implantable element for actively stretching a stomach wall of the patient to create a feeling of satiety.

The term “body tissue” referred to in the present disclosure may be one or several body tissue groups or layers in a patient, such as muscle tissue, connective tissue, bone, etc.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, a connecting portion configured to be placed through a hole in the tissue portion extending between the first and second sides of the tissue portion, the connecting portion having a third cross-sectional area in a third plane and being configured to connect the first portion to the second portion, and a hermetic seal arrangement configured to enclose the connecting portion so as to prevent fluid from the patient to enter the connecting portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, and the connecting portion comprises a flexible structure enabling the connecting portion to flex.

In some embodiments, the flexible structure is configured to allow the connecting portion to flex in more than one direction.

In some embodiments, the flexible structure is configured to allow the connecting portion to flex in all directions.

In some embodiments, the flexible structure comprises a bellows.

In some embodiments, the bellows is a metallic bellows.

In some embodiments, the metallic bellows is welded.

In some embodiments, the bellows is a titanium bellows.

In some embodiments, the bellows form part of the hermetic seal arrangement.

In some embodiments, the flexible structure comprises elevated and lowered portions enabling said flexing of the connecting portion.

In some embodiments, the elevated and lowered portions are configured to enable the connecting portion to be compressed and/or expanded.

In some embodiments, the flexible structure has a substantially cylindrical shape.

In some embodiments, the flexible structure is configured to seal against the first portion and/or the second portion.

In some embodiments, the connecting portion and the second portion are hermetically sealed from the first portion.

In some embodiments, the hermetic seal arrangement encloses the connecting portion and the second portion so as to hermetically seal the connecting portion and the second portion from the first portion.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, wherein the connecting portion and the second portion are configured to form a unit having a central axis extending from a first end of said unit to a second end of said unit, the first end being proximal to the first portion and the second end being distal to the first portion, wherein a physical footprint of said unit perpendicular to the central axis decreases continuously or stepwise from the first end to the second end of said unit.

In some embodiments, said physical footprint comprises a cross-sectional area perpendicular to the central axis.

In some embodiments, the connecting portion and the second portion are one of: configured to reversibly connect to each other to form said unit; or configured to irreversibly connect to each other to form said unit; or configured as a single body forming said unit.

In some embodiments, said unit comprises an angled section forming a bend in said unit.

In some embodiments, the bend is between 15° and 165°, such as between 30° and 150°, such as between 45° and 135°, such as substantially 90°.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, wherein the connecting portion is configured to extend between the first portion and the second portion along a central extension axis, and wherein the second portion is configured to extend in a length direction being divergent with the central extension axis, and wherein the connecting portion has a decreasing cross-sectional area in a direction from the first portion towards the second portion along the central extension axis, wherein a largest cross-sectional area of the second portion in the length direction is smaller than a smallest cross-sectional area of the connecting portion in said direction from the first portion towards the second portion along the central extension axis, and wherein the second portion further has a decreasing cross-sectional area in the length direction from a first end of the second portion proximal to the connecting portion to a second end of the second portion distal to the connecting portion.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, a connecting portion configured to be placed through a hole in the tissue portion extending between the first and second sides of the tissue portion, the connecting portion having a third cross-sectional area in a third plane and being configured to connect the first portion to the second portion, and an electric motor, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, at least part of the electric motor is arranged within the connecting portion.

In some embodiments, the electric motor is arranged within the connecting portion within an imaginary boundary defined by the first surface of the first portion extending through the connecting portion.

In some embodiments, the electric motor is arranged within the connecting portion within an imaginary boundary defined by the second surface of the second portion extending through the connecting portion.

In some embodiments, the electric motor is fully arranged in the connecting portion within imaginary boundaries defined by the first surface of the first portion extending through the connecting portion and the second surface of the second portion extending through the connecting portion respectively.

In some embodiments, the electric motor is arranged such that its longest dimension extends in a direction substantially perpendicular to the first, second and third cross-sectional areas.

In some embodiments, the electric motor is arranged such that its longest dimension extends in a direction between the first portion and the second portion.

In some embodiments, the worm drive is configured to transfer mechanical force from the electric motor to an implantable body engaging portion being external to the implantable energized medical device.

In some embodiments, the electric motor extends through the connecting portion into the first portion and/or the second portion.

In some embodiments, the electric motor extends through an imaginary boundary defined by the first surface of the first portion extending through the connecting portion.

In some embodiments, the electric motor extends through an imaginary boundary defined by the second surface of the second portion extending through the connecting portion.

In some embodiments, the electric motor extends through imaginary boundaries defined by the first surface of the first portion extending through the connecting portion and the second surface of the second portion extending through the connecting portion respectively.

In some embodiments, the implantable energized medical device further comprises a gear arrangement operatively connected to the electric motor wherein the gear arrangement is partly or fully arranged in one of the first portion and the second portion.

In some embodiments, the gear arrangement is arranged within the connecting portion within an imaginary boundary defined by the first surface of the first portion extending through the connecting portion.

In some embodiments, the gear arrangement is arranged within the connecting portion within an imaginary boundary defined by the second surface of the second portion extending through the connecting portion.

In some embodiments, the gear arrangement is fully arranged in the connecting portion within imaginary boundaries defined by the first surface of the first portion extending through the connecting portion and the second surface of the second portion extending through the connecting portion respectively.

In some embodiments, the gear arrangement extends through the connecting portion into the first portion and/or the second portion.

In some embodiments, the gear arrangement extends through an imaginary boundary defined by the first surface of the first portion extending through the connecting portion.

In some embodiments, the gear arrangement extends through an imaginary boundary defined by the second surface of the second portion extending through the connecting portion.

In some embodiments, the gear arrangement extends through imaginary boundaries defined by the first surface of the first portion extending through the connecting portion and the second surface of the second portion extending through the connecting portion respectively.

In some embodiments, the gear arrangement is configured to transfer mechanical force from the electric motor to an implantable body engaging portion being external to the implantable energized medical device.

In some embodiments, the gear arrangement is a worm drive or comprises a worm drive.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion comprising a first surface configured to face a first tissue surface of the first side of the tissue portion, the first portion being further configured to connect, directly or indirectly, to a second portion placed on a second side of the tissue portion opposing the first side, wherein the first portion comprises an internal wireless energy transmitter configured to transmit energy wirelessly to the second portion.

In some embodiments, the first portion is configured to connect, directly or indirectly, to the second portion, via a connecting portion configured to extend through a hole in the tissue portion, the hole extending between the first side of the tissue portion and the second side of the tissue portion.

In some embodiments, the implantable energized medical device further comprises the connecting portion.

In some embodiments, the connecting portion is integrally formed with the first portion.

In some embodiments, the connecting portion is a separate component with regard to the first portion, the connecting portion being configured to be connected to the first portion.

In some embodiments, the first portion has a first cross-sectional area in a first plane and the connecting portion has a second cross-sectional area in a second plane, wherein the first and second planes are parallel to each other, wherein the second cross-sectional area is smaller than the first cross-sectional area, such that the first portion and the second portion are prevented from travelling through the hole in the tissue portion in a direction perpendicular to the first and second planes.

In some embodiments, the first portion is configured to detachably connect, directly or indirectly, to the second portion.

In some embodiments, the first portion comprises a first wireless energy receiver for receiving energy transmitted wirelessly by an external wireless energy transmitter.

In some embodiments, the first portion comprises a first energy storage unit connected to the first wireless energy receiver.

In some embodiments, the first energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to a second wireless energy receiver in the second portion.

In some embodiments, the first portion comprises a first controller comprising at least one processing unit.

In some embodiments, the first controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the connecting portion comprises a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the first portion is excentric with respect to the first portion.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion, and—a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; an implantable reservoir configured to hold a fluid; an implantable pump configured to transfer fluid to and from the implantable reservoir and the body engaging implant respectively via a conduit; and an implantable electric motor connected to the implantable energy storage unit, the implantable electric motor being configured to operate the implantable pump; wherein the implantable energy storage unit, the implantable reservoir, the implantable pump and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable energy storage unit arranged in the first portion, the connecting portion or the second portion; wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable reservoir configured to hold a fluid; an implantable pump configured to transfer fluid to and from the implantable reservoir and the body engaging implant respectively via a conduit; and an implantable electric motor connected to the implantable energy storage unit, the implantable electric motor being configured to operate the implantable pump; wherein the implantable reservoir, the implantable pump and the implantable electric motor are arranged externally to the implantable energized medical device.

A system is provided, the system comprising
an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable electric motor arranged in the first portion, the connecting portion or the second portion; wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; an implantable reservoir configured to hold a fluid; an implantable pump configured to transfer fluid to and from the implantable reservoir and the body engaging implant respectively via a conduit; wherein the implantable electric motor is connected to the implantable energy storage unit, and the implantable electric motor is configured to operate the implantable pump; wherein the implantable energy storage unit, the implantable reservoir and the implantable pump are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable reservoir configured to hold a fluid, the implantable reservoir being arranged in the first portion, the connecting portion or the second portion wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes a body engaging implant configured to at least one of stretch, contract, expand, stimulate, and exert a force on body tissue or a body organ; an implantable energy storage unit; an implantable pump configured to transfer fluid to and from the reservoir and the body engaging implant respectively via a conduit; and an implantable electric motor connected to the implantable energy storage unit, the implantable electric motor being configured to operate the implantable pump; wherein the implantable energy storage unit, the implantable pump and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable pump arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; an implantable reservoir configured to hold a fluid; and an implantable electric motor connected to the implantable energy storage unit, the implantable electric motor being configured to operate the implantable pump; wherein the implantable pump is configured to transfer fluid to and from the implantable reservoir and the body engaging implant respectively via a conduit; wherein the implantable energy storage unit, the implantable reservoir, and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion, and—a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; and an implantable electric motor connected to the implantable energy storage unit, the implantable electric motor being configured to operate the body engaging implant; wherein the implantable energy storage unit and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable energy storage unit arranged in the first portion, the connecting portion or the second portion, and—an implantable electric motor arranged in the first portion, the connecting portion or the second portion, the implantable electric motor being connected to the implantable energy storage unit, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable reservoir configured to hold a fluid; and an implantable pump configured to transfer fluid to and from the implantable reservoir and the body engaging implant respectively via a conduit; wherein the implantable electric motor is configured to operate the implantable pump; wherein the implantable reservoir and the implantable pump are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable reservoir configured to hold a fluid arranged in the first portion, the connecting portion or the second portion, and—an implantable electric motor arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit, and an implantable pump configured to transfer fluid to and from the implantable reservoir and the body engaging implant respectively via a conduit; wherein the implantable electric motor is connected to the implantable energy storage unit and configured to operate the implantable pump; wherein the implantable energy storage unit and the implantable pump are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable reservoir configured to hold a fluid, the implantable reservoir being arranged in the first portion, the connecting portion or the second portion, and—an implantable pump arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; and an implantable electric motor connected to the implantable energy storage unit, the implantable electric motor being configured to operate the implantable pump; wherein the implantable pump is configured to transfer fluid to and from the implantable reservoir and the body engaging implant respectively via a conduit; and wherein the implantable energy storage unit and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable pump arranged in the first portion, the connecting portion or the second portion, and—an implantable energy storage unit arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable reservoir configured to hold a fluid; an implantable electric motor connected to the implantable energy storage unit, the implantable electric motor being configured to operate the implantable pump; wherein the implantable pump is configured to transfer fluid to and from the implantable reservoir and the body engaging implant respectively via a conduit wherein the implantable reservoir and the implantable electric motor are arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable electric motor arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit; wherein the implantable electric motor is connected to the implantable energy storage unit, the implantable electric motor being configured to operate the body engaging implant; wherein the implantable energy storage unit is arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable energy storage unit arranged in the first portion, the connecting portion or the second portion,—an implantable electric motor arranged in the first portion, the connecting portion or the second portion, the implantable electric motor being connected to the implantable energy storage unit, and—an implantable reservoir configured to hold a fluid, the implantable reservoir being arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable pump configured to transfer fluid to and from the implantable reservoir and the body engaging implant respectively via a conduit; wherein the implantable electric motor is configured to operate the implantable pump; wherein the implantable pump is arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable reservoir configured to hold a fluid arranged in the first portion, the connecting portion or the second portion,—an implantable electric motor arranged in the first portion, the connecting portion or the second portion, and—an implantable pump arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable energy storage unit, and wherein the implantable pump is configured to transfer fluid to and from the implantable reservoir and the body engaging implant respectively via a conduit; wherein the implantable electric motor is connected to the implantable energy storage unit and configured to operate the implantable pump; wherein the implantable energy storage unit is arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable reservoir configured to hold a fluid, the implantable reservoir being arranged in the first portion, the connecting portion or the second portion,—an implantable pump arranged in the first portion, the connecting portion or the second portion, and—an implantable energy storage unit arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable electric motor connected to the implantable energy storage unit, the implantable electric motor being configured to operate the implantable pump; wherein the implantable pump is configured to transfer fluid to and from the implantable reservoir and the body engaging implant respectively via a conduit; and wherein the implantable electric motor is arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion,—an implantable pump arranged in the first portion, the connecting portion or the second portion,—an implantable energy storage unit arranged in the first portion, the connecting portion or the second portion, and—an electric motor arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable reservoir configured to hold a fluid; wherein the implantable electric motor is connected to the implantable energy storage unit, the implantable electric motor being configured to operate the implantable pump; wherein the implantable pump is configured to transfer fluid to and from the implantable reservoir and the body engaging implant respectively via a conduit wherein the implantable reservoir is arranged externally to the implantable energized medical device.

A system is provided, the system comprising an implantable energized medical device configured to be held in position by a tissue portion of a patient, the medical device comprising:—a first portion configured to be placed on a first side of the tissue portion, the first portion having a first cross-sectional area in a first plane and comprising a first surface configured to face a first tissue surface of the first side of the tissue portion,—a second portion configured to be placed on a second side of the tissue portion, the second side opposing the first side, the second portion having a second cross-sectional area in a second plane and comprising a second surface configured to engage a second tissue surface of the second side of the tissue portion,—a connecting portion configured to be placed through a hole in the tissue portion extending between the first and second sides of the tissue portion, the connecting portion having a third cross-sectional area in a third plane and being configured to connect the first portion to the second portion, and—an implantable energy storage unit arranged in the first portion, the connecting portion or the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes; a body engaging implant configured to at least one of stretch, contract, expand, stimulate, and exert a force on body tissue or a body organ; an implantable electric motor connected to the implantable energy storage unit, the implantable electric motor being configured to operate the body engaging implant; wherein the implantable electric motor is arranged externally to the implantable energized medical device.

In some embodiments, the implantable energized medical device further comprises a first wireless communication receiver configured to receive communication signals from outside the patient's body.

In some embodiments, the implantable energized medical device further comprises a second wireless communication transmitter arranged in the second portion, wherein the second wireless communication transmitter is configured to transmit communication signals to the first wireless communication receiver.

In some embodiments, the implantable energized medical device further comprises a first wireless communication transmitter arranged in the first portion, the first wireless communication transmitter being configured to transmit communication signals outside of the patient's body.

In some embodiments, the implantable energized medical device further comprises a second wireless communication receiver arranged in the second portion, wherein the first wireless communication transmitter is configured to transmit communication signals to the second wireless communication receiver.

In some embodiments, the implantable energized medical device further comprises a wireless energy receiver configured to receive energy transmitted wirelessly from outside the patient's body and deliver the received energy to the implantable energy storage unit.

In some embodiments, the implantable energized medical device further comprises a control unit configured to control at least one of the body engaging implant, the implantable energy storage unit, the implantable pump, and the implantable electric motor.

In some embodiments, the implantable electric motor is operatively connected to the implantable pump via a rotatable shaft.

In some embodiments, the implantable electric motor is operatively connected to the implantable pump via a magnetic coupling.

In some embodiments, the system further comprises a gear arrangement arranged in the implantable energized medical device and operatively connected to the electric motor, the gear arrangement being configured to reduce the velocity and increase the force of movement generated by the electric motor.

In some embodiments, the system further comprises a gear arrangement arranged externally to the implantable energized medical device and operatively connected to the electric motor, the gear arrangement being configured to reduce the velocity and increase the force of movement generated by the electric motor.

In some embodiments, the system further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the system further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the system further comprises a gear arrangement, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the pump is an hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion is configured to be placed subcutaneously in the patient, and wherein the first portion comprises a connecting interface arrangement for transferring wired energy and/or wired communication signals and/or fluid to an additional implant in the patient.

In some embodiments, a height of the first portion measured in a plane perpendicular to the first plane is 15 mm or less, such as 10 mm or less, such as 7 mm or less, such as 5 mm or less.

In some embodiments, the connecting interface arrangement comprises a port for transferring fluid from the first portion to said additional implant.

In some embodiments, the implantable energized medical device further comprises at least one conduit or tube for transferring said fluid, wherein the at least one conduit or tube is connected to the port.

In some embodiments, the implantable energized medical device further comprises at least one wire for energy and/or communication signals connected to the connecting interface arrangement.

In some embodiments, the height of the first portion is a maximum height.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes, the first portion and the second portion are configured to be placed subcutaneously in the patient, such that the implantable energized medical device can be placed with either of the first portion and the second portion on the first side of the tissue portion.

In some embodiments, a height of the second portion measured in a plane perpendicular to the second plane is 15 mm or less, such as 10 mm or less, such as 7 mm or less, such as 5 mm or less.

In some embodiments, the first portion has a length in a plane parallel to the first plane, wherein the second portion has a length in a plane parallel to the second plane, and wherein the length of the first portion differ no more than 30% with regard to the length of the second portion, such as wherein the length of the first portion differ no more than 15% with regard to the length of the second portion, such as wherein the length of the first portion differ no more than 5% with regard to the length of the second portion, such as wherein the length of the first portion differ no more than 1% with regard to the length of the second portion.

In some embodiments, the first portion has a width in a plane parallel to the first plane, wherein the second portion has a width in a plane parallel to the second plane, and wherein the width of the first portion differ no more than 30% with regard to the width of the second portion, such as wherein the width of the first portion differ no more than 15% with regard to the width of the second portion, such as wherein the width of the first portion differ no more than 5% with regard to the width of the second portion, such as wherein the width of the first portion differ no more than 1% with regard to the width of the second portion.

In some embodiments, the first portion has a height in a plane perpendicular to the first plane, and wherein the height of the first portion differ no more than 30% with regard to the height of the second portion, such as wherein the height of the first portion differ no more than 15% with regard to the height of the second portion, such as wherein the height of the first portion differ no more than 5% with regard to the height of the second portion, such as wherein the height of the first portion differ no more than 1% with regard to the height of the second portion.

In some embodiments, a height of the first portion measured in a plane perpendicular to the first plane is 15 mm or less, such as 10 mm or less, such as 7 mm or less, such as 5 mm or less.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first portion comprises a first energy storage unit connected to the first wireless energy receiver.

In some embodiments, the second portion comprises a second energy storage unit connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement and an electric motor, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

In some embodiments, the second portion comprises at least one hydraulic pump.

In some embodiments, the hydraulic pump comprises a pump comprising at least one compressible hydraulic reservoir.

According to an embodiment of the inventive concept, an implantable energized medical device configured to be held in position by a tissue portion of a patient is provided, the medical device comprising: a first portion configured to be placed on a first side of the tissue portion, the first portion having a first cross-sectional area in a first plane and comprising a first surface configured to face a first tissue surface of the first side of the tissue portion, a second portion configured to be placed on a second side of the tissue portion, the second side opposing the first side, the second portion having a second cross-sectional area in a second plane and comprising a second surface configured to engage a second tissue surface of the second side of the tissue portion, and a connecting portion configured to be placed through a hole in the tissue portion extending between the first and second sides of the tissue portion, the connecting portion having a third cross-sectional area in a third plane and being configured to connect the first portion to the second portion, wherein the second portion comprises or forms a reservoir for holding a fluid; the implantable energized medical device further comprising: a sealed container configured to protrude into the reservoir; an actuator connected to the sealed container, the actuator being configured to expand or retract the sealed container to change the volume of the sealed container for pumping fluid to or from the reservoir; wherein: the first, second, and third planes are parallel to each other, the third cross-sectional area is smaller than the first and second cross-sectional areas, such that the first portion and second portion are prevented from travelling through the hole in the tissue portion in a direction perpendicular to the first, second and third planes.

In some embodiments, the actuator comprises an electric motor.

In some embodiments, the actuator is arranged in the connecting portion.

In some embodiments, the actuator is partly or fully arranged inside the sealed container.

In some embodiments, the second portion comprises a port in fluid communication with the reservoir for transferring fluid between the reservoir and an additional implant in the patient.

In some embodiments, the implantable energized medical device further comprises a conduit connected to the port, the conduit being configured to transfer fluid between the reservoir and the additional implant.

In some embodiments, the implantable energized medical device further comprises an injection port for introducing fluid, the injection port being arranged in the first portion.

In some embodiments, the implantable energized medical device further comprises an internal conduit connecting the injection port to the reservoir.

In some embodiments, the sealed container is a bellows.

In some embodiments, the bellows is a metallic bellows.

In some embodiments, at least a portion of the sealed container configured to be in contact with fluid comprises metal.

In some embodiments, the volume of the sealed container can be altered such that the volume of the sealed container is more than 60% of the maximum volume of the reservoir.

In some embodiments, the sealed container comprises at least one flexible portion, and wherein the flexible portion enable at least one of compression and expansion of the sealed container.

In some embodiments, the sealed container comprises at least one elastic portion, and wherein the elastic portion enable at least one of compression and expansion of the sealed container.

In some embodiments, the implantable energized medical device further comprises a first energy storage unit and/or a second energy storage unit for powering the actuator.

In some embodiments, the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.

In some embodiments, the first energy storage unit is connected to the first wireless energy receiver.

In some embodiments, the second portion comprises the second energy storage unit, wherein the second energy storage unit is connected to the second wireless energy receiver.

In some embodiments, at least one of the first and second energy storage unit is a solid-state battery.

In some embodiments, the solid-state battery is a thionyl-chloride battery.

In some embodiments, the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.

In some embodiments, the first portion comprises a first controller comprising at least one processing unit.

In some embodiments, the second portion comprises a second controller comprising at least one processing unit.

In some embodiments, the first controller and/or the second controller is configured to control the actuator.

In some embodiments, at least one of the first and second controller is connected to a wireless transceiver for communicating wirelessly with an external device.

In some embodiments, the first controller is connected to a first wireless communication receiver in the first portion for receiving wireless communication from an external device, the first controller is connected to a first wireless communication transmitter in the first portion for transmitting wireless communication to a second wireless communication receiver in the second portion.

In some embodiments, the second controller is connected to the second wireless communication receiver for receiving wireless communication from the first portion.

In some embodiments, the first wireless energy receiver comprises a first coil and the wireless energy transmitter comprises a second coil.

In some embodiments, the first portion comprises a combined coil, wherein the combined coil is configured to receive energy wirelessly from an external wireless energy transmitter, and transmit energy wirelessly to the second wireless receiver of the second portion.

In some embodiments, at least one of the coils are embedded in a ceramic material.

In some embodiments, the implantable energized medical device further comprises a housing configured to enclose at least the first portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the implantable energized medical device further comprises a housing configured to enclose at least the second portion, and wherein a first portion of the housing is made from titanium and a second portion of the housing is made from a ceramic material.

In some embodiments, the portion of the housing made from a ceramic material comprises at least one coil embedded in the ceramic material.

In some embodiments, the first portion is detachably connected to at least one of the second portion and the connecting portion.

In some embodiments, the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.

In some embodiments, a connecting interface between the connecting portion and the second portion is excentric with respect to the second portion.

In some embodiments, a connecting interface between the connecting portion and the first portion is excentric with respect to the first portion.

In some embodiments, the second portion has a first end and a second end opposing the first end, wherein the second portion has a length between the first and second end.

In some embodiments, the first end and second end are separated in a direction parallel to the second plane.

In some embodiments, the first and second ends comprise an elliptical point respectively.

In some embodiments, the first and second ends comprise a hemispherical end cap respectively.

In some embodiments, the second portion has at least one circular cross-section along the length between the first and second end.

In some embodiments, the second portion has at least one oval cross-section along the length between the first and second end.

In some embodiments, the second portion has at least one elliptical cross-section along the length between the first and second end.

In some embodiments, the implantable energized medical device further comprises a gear arrangement, wherein the gear arrangement is configured to reduce the velocity and increase the force of the movement generated by the electric motor.

In some embodiments, the gear arrangement is configured to transfer a force with a high velocity into a stronger force with lower velocity.

In some embodiments, the gear arrangement is configured to transfer a rotating force into a linear force.

In some embodiments, the gear arrangement comprises a gear system.

In some embodiments, the second portion comprises a magnetic coupling for transferring mechanical work from the electric motor through one of: a barrier separating a first chamber of the second portion from a second chamber of the second portion, a housing enclosing at least the second portion.

### B: Artificial Intestine Sections

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating artificial intestine sections, examples of which will now be described.

The essence of the present disclosure relating to an artificial intestine section provides a system for treating a patient having a disorder related to the patient's intestine. The system comprises an artificial intestine section adapted to being implanted inside a patient's body, preferably along with an accumulator for accumulating energy. The intestine section may have a first open end portion and a second open end portion in flow communication with one another, wherein at least the first open end portion and possibly also the second open end portion is adapted to being connected to a surgically created opening in the patient's intestine. Furthermore, the accumulator may be adapted to be charged wirelessly with energy and to be arranged so as to supply energy directly or indirectly to at least one energy consuming part of said artificial intestine section.

### Lateral Connection to Intestine

Preferably, at least the first open end portion and possibly also the second open end portion of the artificial intestine section is adapted to being connected to a lateral opening surgically created in a wall of the patient's intestine. Thus, rather than connecting the artificial intestine section to the cross-sectional end of the patient's intestine, it is connected to a lateral opening in the patient's intestinal wall, and for this purpose the respective open end portion of the artificial intestine section is specifically adapted.

By connecting the artificial intestine section laterally to the intestine, forces caused by the peristaltic movement of the intestine and acting on the artificial intestine section of the intestine are largely avoided. More specifically, where the artificial intestine section is connected to the cross-sectional opening of the intestine, the peristaltic waves of the intestine tend to pull the intestine off of the connection between the intestine and the artificial intestine section. As compared to this, where the artificial intestine section is attached to an opening in the lateral wall of the intestine, the peristaltic waves pass the artificial intestine section substantially without any impact on the connection between the intestinal wall and the artificial intestine section.

### Structure of Lateral Attachment

In order to securely attach the artificial intestine section to the lateral opening, at least the first open end portion may comprise a shoulder portion formed around the end portion for lateral connection to the patient's intestinal wall. Preferably, at least a part of the shoulder portion extends laterally from the artificial intestine section by 3 mm to 20 mm. Furthermore, the shoulder portion preferably has a curved cross section, so as to generally conform to the intestinal wall when laterally attached thereto. An open end portion adapted in this way can advantageously be attached to the intestinal wall from the outside thereof.

According to an improved embodiment, the shoulder portion may be split into an upper and a lower shoulder portion with a gap between the upper and lower shoulder portions for accommodating intestinal wall tissue therein. The lower shoulder portion, if suitably adapted, can then be placed inside the patient's intestine through the surgically created lateral wall opening, whereas the upper shoulder portion will be placed outside the intestinal wall.

In order to allow the lateral wall opening to be easily stretched over the lower shoulder portion when the lower shoulder portion is advanced there through and yet in order to have a large contact area between the intestinal wall and the shoulder portion, the upper shoulder portion may be made larger than the lower shoulder portion. Thus, the surface area of the upper shoulder portion contacting the intestinal wall is also larger than the surface area of the lower shoulder portion contacting the intestinal wall.

The open end portion for lateral connection to the patient's intestinal wall may be adapted to being connected to the patient's intestinal wall by gluing. For instance, it may have a particular rough surface structure for the glue to better adhere. Also, the open end portion may be adapted to being connected to the patient's intestinal wall by sewing. For instance, a certain area of the shoulder portion may be perforated for stitching through the perforations or may be made from a material which is easy to penetrate with a needles. Similarly, the open end portion may specifically be adapted to being connected to the patient's intestinal wall by stapling.

### Frontal Connection to Intestine

While it is generally preferred to connect the first and possibly also the second open end portion of the artificial intestine section laterally to an opening in the patient's intestinal wall, it is as well possible to adapt the open end portion for being connected to a cross-sectional opening surgically created in the patient's intestine.

### Structure of Frontal Attachment (Bulge/Sleeve)

In this case, the open end portion of the artificial intestine section preferably comprises
a conduit having an outer surface with at least one bulge extending outwardly from the conduit's outer surface in a circumferential direction of the conduit about at least a part of the conduit's circumference, and
a blocking ring loosely fitting over the outer surface of the conduit with a clearance between the outer surface and the blocking ring for mounting intestinal tissue within the clearance, said blocking ring having an inner cross sectional diameter which is smaller than or substantially identical to an outer cross sectional diameter of the at least one bulge so as to prevent the blocking ring from slipping over the bulge when intestinal tissue is mounted within the clearance.

The artificial intestine section may then be affixed to the cross-sectional opening of the intestine part by inserting the artificial intestine section having a bulge formed on the outside thereof into the cross-sectional opening of the intestine part so that the intestine part extends over the bulge from one side of the bulge and advancing a blocking ring over the intestine part towards the bulge from the respective other side of the bulge such that the intestine part is located intermediate the outer surface of the artificial intestine section and the blocking ring.

Alternatively, the open end portion may comprise a conduit having an outer surface and a flexible sleeve adapted to axially extend and closely fit around at least part of said outer surface of the conduit. The flexible sleeve can be mounted on said outer surface either folded or rolled upon itself or so as to be foldable upon itself. In either case, the open end portion of the artificial intestine section may then be affixed to the cross-sectional opening of the intestine part by inserting the open end portion of the artificial intestine section into the cross-sectional opening of the intestine part and placing the flexible sleeve so as to extend over both the intestine part and open end portion of the artificial intestine section such that the intestine part is located intermediate the sleeve and the outer surface of the artificial intestine section.

Where the flexible sleeve is mounted on the outer surface of the open end portion of the artificial intestine piece so as to be foldable upon itself, the step of placing the flexible sleeve so as to extend over both the intestine part and open end portion of the artificial intestine section comprises folding the flexible sleeve upon itself such that the intestine part is located intermediate the folded sleeve.

The bulge and sleeve may also be provided on one conduit so as to be used in combination.

The afore-mentioned conduit of the artificial intestine section with a sleeve or with a bulge serve to improve the strength of the connection against axial forces which may e.g. result from the peristaltic movement of the intestine and tend to pull on the intestine.

Furthermore, the conduit of the open end portion preferably comprises a multilayer material. For instance, it is advantageous when the open end portion comprises a porous ingrowth layer that allows ingrowth of living tissue. The ingrowth layer may have a net-like structure and is most preferably made from Dacron®.

### Through-Flow Arrangement

According to the afore-described structures of the first and second open end portions of the artificial intestine section, it is possible to connect both the first and second open end portions to a surgically created cross-sectional opening in the patient's intestine, so as to form an intermediate intestine section, or to connect both the first and second open end portions to a surgically created lateral opening in a wall of the patient's intestine, so as to form a by-pass intestine section.

Alternatively, the second open end portion may be adapted to being connected to a surgically created stoma or to the patient's rectum or anus or to tissue adjacent the patient's anus, so as to form an intestine end section.

Further alternatively, the second open end portion may be adapted to being connected to a portion of the patient's small intestine or to a portion of the patient's large intestine, as the case may be, and this portion of the patient's intestine may then lead to the surgically created stoma or to the patient's rectum or anus or to tissue adjacent the patient's anus.

### Material

Preferably, at least the first open end portion is made from a biocompatible material. The biocompatible material of the open end portion may comprise at least one material of the following group of materials: titanium, stainless steel, ceramics, bio-compatible polymer material. More specifically, the biocompatible polymer material may comprise at least one polymer of the following group of polymers: polytetrafluoroethylene, silicone, polyurethane, expanded polytetrafluoroethylene (ePTFE).

Intest. Content Interacting Device as an Energy Consuming Part

In a preferred embodiment, the at least one energy consuming part of the artificial intestine section comprises at least one element adapted to directly or indirectly interact with intestinal contents contained in the artificial intestine section between the first and second open end portions thereof.

### Flow Control Device as an Energy Consuming Part

In a more advanced embodiment, the at least one element may comprise a flow control device adapted to control flow of intestinal contents from the artificial intestine section through the second open end portion. The flow control device is preferably adapted to prevent flow of intestinal contents from the artificial intestine section through the second open end portion.

### Exit Valve as Flow Control Device

The flow control device preferably comprises at least one valve, including an exit valve preventing intestinal contents flow through the second open end portion in its closed position. Preferably, the exit valve is a normally closed valve so that no energy is needed to keep the valve closed during the system's inactive periods.

### Entry Valve as an Addititonal Part of the Flow Control Device

In addition, the flow control device may comprise an entry valve allowing intestinal contents to flow towards the reservoir in its open position. This can be advantageous particularly during the emptying of the reservoir, when the entry valve should be closed. Therefore, the entry valve is preferably a normally open valve. Accordingly, the exit valve and the entry valve are preferably adapted to cooperate such that when one of the two valves is closed, the respective other valve is open, and vice versa.

### Valve Types

As regards the various valve types that may be employed, the at least one valve may e.g. comprise a compartment with a variable volume adapted to open and close the valve by changing the compartment's volume. Advantageously, the at least one valve comprises at least one passage for filling and emptying the compartment with hydraulic fluid. The compartment preferably has at least one flexible wall defining an opening for a conduit to pass through, the opening being adapted to close upon increase of the compartment's volume.

According to a different embodiment, the at least one valve may be a flap valve. The flap valve may for instance comprise a rotatable disc.

Extra Valve Separate from Artificial Intestine Piece

While the valve or valves preferably make an integral part of the artificial intestine section, the artificial intestine section may further comprise one or more extra valves adapted to control flow of intestinal contents in a natural section of the patient's intestine upstream and/or downstream the artificial intestine section. The extra valve may be rigidly connected to the artificial section but may as well form a completely separate part. The extra valve is adapted to being implanted inside the patient's body outside a section of the patient's natural intestine and comprises at least one element adapted to act on the natural intestine section from the outside thereof so as to prevent intestinal contents flow through the natural intestine section. This valve arrangement does not require any surgery on the respective part of the natural intestine when the valve is implanted.

The extra valve may comprise at least one electrical stimulation device adapted to electrically stimulate muscle or neural tissue of an intestine section so as to cause at least partial contraction of the natural intestine section. This is a very gender way of constricting the intestine. The stimulation device preferably comprises at least one electrode adapted to apply electric pulses to the natural intestine section.

It is particularly advantageous to make use of a stimulation device which is adapted to stimulate different portions of the intestine section over time. Thus, different portions of the intestine section can be constricted by stimulation at different times in any predetermined stimulation pattern, thereby giving the intestine portions currently not stimulated time to recover and, thus, improving the blood circulation in the respective intestine section.

Furthermore, the stimulation device can specifically be adapted to stimulate, over time, the different portions of the intestine section in a wave like manner in a direction opposite to natural intestinal contents flow. As a result, the valve counteracts the natural intestinal contents flow, thereby improving the valve's closing function.

Alternatively, or preferably in addition to the stimulation device, the at least one valve may comprise a constriction device implanted in the patient's body for at least partly constricting the natural intestine section mechanically from outside the natural intestine section. Where the stimulation device is combined with the constriction device, the stimulation device and the constriction device preferably act on the same intestine section. In that case, it is advantageous if the constriction device in its normal condition constricts the natural intestine section only partly, in order not to damage the intestine overtime. Complete constriction and, thus, closing of the intestine may then be obtained by additionally stimulating the natural intestine section in a manner as described before.

In addition, when constriction of the intestine section caused by the constriction device is released, the stimulation device may, if accordingly adapted, be used to pump intestinal contents along the natural intestine section by, over time, stimulating the different portions of the natural intestine section in a wave like manner in a direction of natural intestinal contents flow. In this situation, the valve may incorporate the additional function of a pump for actively supporting the discharge of feces from the human body.

### Pump as an Energy Consuming Part

The at least one energy consuming part of the artificial intestine section may comprise a pump for advancing intestinal contents through the second open end portion to outside the artificial intestine section.

Pump with Reservoir

In addition to the pump, the artificial intestine section may comprise a reservoir between the first and second open end portions for receiving and temporarily collecting therein intestinal contents supplied through the first open end portion. In this case, the pump is preferably adapted for emptying the reservoir through the second open end portion. A variety of different structures may be realized.

For instance, the reservoir may be formed by a bellow, said bellow having an end wall closing the bellow at one end thereof. The end wall may then make part of the pump such that a volume of the bellow is reduced upon advancement of said end wall. Preferably, bellow is made of a resilient material so as to urge the bellow into a normally expanded position.

In another embodiment, the pump may comprise a movable piston, with a front end of the piston extending into the reservoir such that a volume of the reservoir is reduced upon advancement of the piston. Preferably, the piston is spring loaded so as to urge the piston into a normally retracted position.

Alternatively, the pump may be adapted for being permanently arranged inside the reservoir.

In a further alternative, the reservoir may have a flexible wall and the pump is adapted for emptying the reservoir by squeezing the reservoir. In this case, the pump may e.g. include a constriction device adapted to alternately constrict and release sections of the reservoir so as to pump intestinal contents along the reservoir by, over time, constricting different sections of the reservoir in a wave like manner. More specifically, the reservoir may have a tube-like form and a roller pump may be used as the pump acting on the tube-like reservoir from the outside thereof.

### Motor as an Energy Consuming Part

Where the valves or pump or any other energy consuming part of the artificial intestine section is not or not only manually drivable, the artificial intestine section may comprise at least one motor arranged for automatically driving at least one energy consuming part of the artificial intestine section. The motor is preferably arranged to be driven by electric or electromagnetic energy.

A motor in the sense of the present disclosure is a device that transforms energy other than mechanical energy into mechanical energy. While a pump in the sense of the present disclosure is a device for advancing liquid or pasty material, a pump may at the same time be a motor in certain circumstances, such as where the transformation of energy into mechanical energy causes advancement of the liquid or pasty material without any intervening mechanical means such as a piston, bellow or the like.

For instance, the at least one motor can be arranged for driving at least one of the valve or valves, respectively, between its closed and open position. Also, the at least one motor can be arranged for driving the pump.

A manually operable switch may be provided for activating the at least one motor, the switch being preferably arranged for subcutaneous implantation so as to be operable from outside the patient's body.

### Energy Transmission

Where energy is not transmitted wirelessly, galvanic coupling elements may be provided at least between the accumulator and the energy consuming part, in particular the motor, for transmitting energy to the motor in contacting fashion.

The energy may alternatively be transmitted wirelessly from the accumulator to the motor. Thus, the energy source may comprise a wireless energy transmitter adapted to wirelessly transmit energy from the accumulator to the energy consuming part.

Preferably, in order to reduce the number of parts and possibly increase the system's efficiency, the energy consuming part, in particular the motor, can be adapted to directly transform the wirelessly transmitted energy from the accumulator into kinetic energy. In the alternative, the energy consuming part will have to comprise a transforming device for transforming the wirelessly transmitted energy from the accumulator into electric energy.

Similarly, the system preferably comprises an implantable energy transforming device for transforming the wirelessly transmitted energy from outside the patient's body into energy to be stored in the accumulator of the implanted system and further comprises a wireless energy transmitter adapted to wirelessly transmit energy from outside the patient's body to said implantable energy transforming device.

It is further preferred to set up the system such that the energy consuming part is driven with the electric energy, as said energy transforming device transforms the wireless energy into the electric energy.

The energy transmitter can be adapted to generate an electromagnetic field, a magnetic field or an electrical field. The wireless energy may be transmitted by the energy transmission device by at least one wireless signal. More specifically, the energy transmitter may be adapted to transmit the energy by at least one wireless energy signal, which may comprise an electromagnetic wave signal, including at least one of an infrared light signal, a visible light signal, an ultra violet light signal, a laser signal, a microwave signal, an X-ray radiation signal, and a gamma radiation signal. Also, the wireless energy signal may comprise a sound or ultrasound wave signal. Furthermore, the wireless energy signal may comprise a digital or analog signal or a combination thereof.

### Energy Transmission Feedback

A feedback subsystem, which can make part of a control device described subsequently, can advantageously be provided to wirelessly send feedback information related to the energy to be stored in the accumulator from inside the human body to the outside thereof. The feedback information is then used for adjusting the amount of wireless energy transmitted by the energy transmitter. Such feedback information may relate to an energy balance which is defined as the balance between an amount of wireless energy received inside the human body and an amount of energy consumed by the at least one energy consuming part. Alternatively, the feedback information may relate to an energy balance which is defined as the balance between a rate of wireless energy received inside the human body and a rate of energy consumed by the at least one energy consuming part.

### Accumulator

The accumulator preferably comprises a rechargeable battery. It may alternatively or in addition comprise a capacitor. The accumulator may be adapted for being implanted inside the patient's body either fixedly connected to the artificial intestine section or distant to the artificial intestine section.

### Primary Energy Source

A primary energy source may be provided for charging the accumulator with energy from outside the patient's body. The primary energy source is preferably adapted to being mounted on the patient's body.

### Control Unit

It is advantageous to provide a control unit adapted to directly or indirectly control one or more elements of the system, such as for controlling opening of the exit valve and/or closing of the entry valve, in particular in a manner such that when one of the two valves is closed, the respective other valve is open, and vice versa. The control unit can also be adapted to control actuation of the pump.

The control unit is preferably operable by the patient, e.g. particularly in order to empty the reservoir.

At least part of the control unit may be adapted to be implantable in the patient's body. For instance, a manually operable switch may be provided for activating the control unit, the switch preferably being arranged for subcutaneous implantation so as to be operable from outside the patient's body. Also, the control unit may comprise a first part adapted for implantation in the patient's body and a second part adapted to cooperate with the first part from outside the patient's body. In this case, the control unit can be adapted to transmit data from the external second part of the control unit to the implanted first part of the control unit in the same manner as energy is transmitted by said wireless energy transmitter from outside the patient's body to said implantable energy transforming device.

That is, the second part of the control unit may be adapted to wirelessly transmit a control signal to the implantable first part of the control unit for controlling the at least one energy consuming part from outside the patient's body. Also, the implantable first part of the control unit may be programmable via the second part of the control unit. Furthermore, the implantable first part of the control unit may be adapted to transmit a feedback signal to the second part of the control unit.

### Sensor

Furthermore, a physical parameter sensor adapted to directly or indirectly sense a physical parameter of the patient can be provided. The physical parameter sensor may be adapted to sense at least one of the following physical parameters of the patient: a pressure within the artificial intestine section, a pressure within the patient's natural intestine, an expansion of the artificial intestine section, a distension of an intestinal wall of the patient's natural intestine, a movement of the patient's intestinal wall.

Similarly, a functional parameter sensor adapted to directly or indirectly sense a functional parameter of the system can be provided, wherein the functional parameter sensor may be adapted to sense at least one of the following functional parameters of the system: a pressure against a part of the system such as the artificial intestine section, a distension of a part of the system such as a wall of the artificial intestine section, an electrical parameter such as voltage, current or energy balance, a position or movement of a movable part of the system.

Preferably, an indicator is coupled to the sensor or sensors, the indicator being adapted to provide a signal when a sensor senses a value for the parameter beyond a predetermined threshold value. The sensor signal may comprise at least one of the following types of signals: a sound signal, a visual signal.

Intest. Content Interacting Device as an Energy Consuming Part (Flow Control Device/Exit Valve/Entry Valve/Pump/Reservoir)

As mentioned before, the artificial intestine section or system may comprise at least one energy consuming part adapted to directly or indirectly interact with intestinal contents contained in the artificial intestine section between the first and second open end portions thereof. This element will be implanted along with the artificial intestine section. As also mentioned before, the energy consuming part may comprise a flow control device adapted to control flow of intestinal contents from the artificial intestine section through the second open end portion.

Again, the flow control device may comprise an exit valve preventing intestinal contents flow through the second open end portion in its closed position and may additionally comprise an entry valve allowing intestinal contents to flow through the first open end portion into the artificial intestine section in its open position.

Alternatively or in addition, as also mentioned before, the flow control device may comprise a pump for advancing intestinal contents through the second open end portion to outside the artificial intestine section.

If a reservoir is provided between the first and second open end portions for receiving and temporarily collecting therein intestinal contents supplied through the first open end portion, the pump may be adapted to empty the reservoir through the second open end portion.

Furthermore, where the pump comprises a manually operable switch for activating the pump, the method of implantation may further comprise the step of implanting the switch subcutaneously so as to be operable from outside the patient's body.

### Motor

Again, at least one motor may be implanted along with the artificial intestine section and may be arranged for automatically driving one or more energy consuming part of the flow control device. Where the motor comprises a manually operable switch for activating the motor, the method of implantation may further comprise the step of implanting the switch subcutaneously so as to be operable from outside the patient's body.

### Energy Transmission

Where energy is transmitted wirelessly, for instance from outside the patient's body to inside the patient's body either to an energy consuming part and/or more specifically to be stored in the accumulator or from the accumulator to the energy consuming part, it may further be necessary to implant an energy transforming device for transforming the wireless energy into electric energy. Alternatively or in addition, galvanic coupling elements may be implanted, e.g. for transmitting energy to the energy consuming part in contacting fashion from the implanted energy source.

### Accumulator

Preferably, at least one rechargeable battery is provided as the accumulator. Alternatively or in addition thereto, at least one capacitor may be provided as the accumulator.

### Control Unit

Furthermore, as mentioned previously, at least a part of a control unit may be implanted inside the patient's body adapted to directly or indirectly control one or more of the elements that have also been implanted in the patient's body. Where the control unit comprises a manually operable switch for activating the control unit, the method of implantation may further comprise the step of implanting said switch subcutaneously so as to be operable from outside the patient's body.

### Sensor

As mentioned before, one or more physical and/or functional parameter sensors may be implanted to directly or indirectly sense physical and/or functional parameters inside the patient and in the system implanted inside the patient. Where the sensor is a pressure sensor, it may be placed in the artificial intestine section or the patient's natural intestine so as to sense the pressure within the artificial intestine section or patient's natural intestine, respectively. Where the sensor is a tension sensor, it may be placed in contact with the artificial intestine section or the patient's natural intestine so as to sense an expansion of the artificial intestine section or patient's natural intestine, respectively. Where the sensor is a movement sensor, it may be placed in contact with the artificial intestine section or the patient's natural intestine so as to sense movement of the artificial intestine section or patient's natural intestine, respectively. The functional sensor may be adapted to measure at least one of the following functional parameters: an electrical parameter such as voltage, current or energy balance or a stimulation parameter in relation to the system.

### C: Intestinal Reservoir with Implantable Flow Control Device

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating implantable flow control devices which control the flow of intestinal contents in relation to an intestinal reservoir, examples of which will now be described.

The essence of the present disclosure relating to an intestinal reservoir with an implantable flow control device provides a system that acts on an intestinal reservoir, i.e. on a reservoir which is formed from surgically modified intestine that has been cut along a mutual contact line of laterally adjacent sections of a bent portion of intestine and connected so that the resulting upper and lower halves of the intestine form an intestinal wall of the reservoir. The system comprises an artificial flow control device implantable in the patient's body and adapted to control flow of the intestinal contents from said reservoir. The flow control device comprises at least one pump adapted to act on said intestinal wall so as to reduce the reservoir's volume in order to empty the reservoir.

Since the reservoir is made from tissue of the intestine, it is not necessary to connect any artificial piece to living tissue of the patient's natural intestine. Rather is the pump adapted to act on the intestinal wall of the reservoir and can therefore be implanted within the patient's body preferably outside the reservoir. Due to the pump being implanted, an external collecting device need not be attached, removed and cleaned when emptying of the reservoir is desired.

Three preferred basic principles of such pump will be described hereinafter, a mechanical type pump, a hydraulic type pump and an electrical stimulation type pump. These pumps can be combined to cooperate, if desired.

### Integrated and Adjacent Location of Pump

According to a first preferred embodiment, components of the pump are adapted for implantation in surgically created folds of said intestinal wall of the reservoir. This is particularly advantageous in relation to the electrical stimulation type pump, as will be described hereinafter.

According to a second preferred embodiment, components of the pump are adapted for implantation inside the patient's body adjacent the reservoir. This is preferable in relation to all three pump types.

In either case, the components of the pump will not come into contact with intestinal contents. However, it is not entirely excluded that a pump is used in context with the present disclosure that is adapted to be at least partly placed inside the intestinal reservoir.

### Electrical Stimulation Type Pump

The electrical stimulation type pump comprises at least one electrical stimulation device adapted to electrically stimulate a muscle or neural tissue of said intestinal wall so as to cause at least partial contraction thereof. This is a very gender way of constricting the reservoir. The stimulation device is preferably adapted to apply electric pulses to the intestinal wall. For this purpose, the stimulation device preferably comprises at least on electrode adapted to generate the electric pulses.

It is particularly advantageous to make use of a stimulation device which is adapted to stimulate different portions of the intestinal wall over time. Thus, different parts of the reservoir can be constricted by stimulation of different intestinal wall portions at different times in any predetermined stimulation pattern. This way, the pump can be adapted to empty the intestinal reservoir by pumping intestinal contents along a section of the reservoir by, over time, stimulating different portions of said intestinal wall in a direction of natural intestinal contents flow.

For this purpose, the pump may comprise a plurality of said electrical stimulation devices with a longitudinal, rod-like or plate like shape. A plurality of electrodes may be arranged in one or more rows along the length of the electrical stimulation devices. The longitudinal stimulation devices can then be arranged side by side with little or no distance, when implanted, so as to cover substantially the entire intestinal reservoir on one side, preferably on two opposing sides, of the reservoir. This structure and arrangement is particularly suitable for advancing intestinal contents through the reservoir in a controlled manner. For instance, beginning at one end of the reservoir, the “entry”, mutually adjacent portions of the intestinal wall and, thus, mutually adjacent sections of the intestinal reservoir can be stimulated consecutively along the length of the reservoir until the respective other end of the reservoir is reached, the “exit”, so that the entire reservoir is in a constricted state at the end of the emptying process. Alternatively, the mutually adjacent portions of the intestinal wall can be stimulated in a wave-like manner, i.e. the preceding portion can relax once the next following portion has been stimulated sufficiently. However, the electrical stimulation devices may likewise be activated all at the same time, thereby simultaneously urging away intestinal contents from all areas of the reservoir. In this case it is particularly important for the system to comprise an entry valve at the upstream end of the intestinal reservoir which has to be closed to prevent back-flow when the electrical stimulation type pump is activated.

The length of the stimulation devices should be sufficient to substantially span the entire width of the intestinal reservoir, in order to facilitate handling and reduce the number of parts. The perfect length depends on the size of the intestinal reservoir, but is preferably more than 10 cm.

Handling of a plurality of electrical stimulation devices can be improved when they are embedded in a flexible web, e.g. integrated within a biocompatible, non-degradable polymer film, such as a polytetrafluoroethylene film. The flexibility of the web allows the electrical stimulation devices to follow movements of the intestinal reservoir, in particular when sections thereof are constricted individually due to selective electric stimulation.

Instead of embedding the longitudinal electrical stimulation devices in a flexible web, they may be adapted for implantation in surgically created folds of the intestinal wall of the reservoir. The open side of the folds may be closed by sewing, bonding and/or stapling the tissue of the intestinal wall together so as to form bags for the longitudinal electrical stimulation devices, either before or preferably after the stimulation devices have been put in place. The stimulation devices need not necessarily be longitudinal but may have any other shape, whereby the folds or bags formed from the intestinal wall are suitably formed to accommodate them.

Alternatively, instead of providing a plurality of longitudinal electrical stimulation devices, the electrical stimulation device may be formed as an integral unit on at least one side of the reservoir. This makes handling and manufacture even easier. However, an integral unit is relatively stiff, if not entirely rigid, which might be less comfortable for the patient as compared to e.g. the afore mentioned flexible web. Preferably, the integral unit has a size sufficiently large to span an area of 10 cm×10 cm or larger.

Independent of whether the electrical stimulation devices are rod-like, plate-like, combined in a flexible web or provided in the form of a stiff or rigid integral unit, they are preferably so adapted that they can be arranged in two planes in order to accommodate the intestinal reservoir between these two planes.

Electrical Stimulation Device Combined with Constriction Device

Alternatively, or preferably in addition to the at least one electrical stimulation device, the pump may comprise a constriction type pump implanted in the patient's body for at least partly constricting the intestinal reservoir mechanically or hydraulically by acting from outside on the intestinal wall. Similar to the above described electrical stimulation type pump, the constriction type pump may be adapted to pump intestinal contents along the reservoir by, over time, constricting different sections of the reservoir consecutively or in a wave-like manner in a direction of natural intestinal contents flow. Where electrical stimulation devices of the electrical stimulation type pump are combined with constriction devices of the constriction type pump, the stimulation devices and the constriction devices preferably act on the same portions of the intestinal wall so as to pump the intestinal contents along the reservoir by, overtime, electrically stimulating different portions of said intestinal wall and simultaneously constricting respective sections of the reservoir in the direction of natural intestinal contents flow. It is advantageous when the constriction type pump in operation constricts the intestinal reservoir only partly, in order not to damage the intestinal tissue. Complete constriction and, thus, emptying of the reservoir may then be obtained by additionally stimulating the intestinal wall portions electrically in a manner as described before.

It should be noted that, due to the surgical modifications, the intestinal reservoir itself has lost its natural peristaltic capabilities. Therefore, according to a preferred embodiment, the electrical stimulation type pump is adapted to pump intestinal contents along the reservoir in a direction of natural intestinal contents flow by, over time, stimulating different portions of the intestinal wall in a wave-like (peristaltic) manner when constriction of the reservoir caused by the constriction type pump is released. Thereby, the filling of the intestinal reservoir with intestinal contents supplied to the reservoir is improved. This is even useful in cases where the electrical stimulation type pump is not combined with a constriction type pump or with any other pump device. In either case, an exit valve should be provided at the downstream end of the intestinal reservoir, which has to be closed while the reservoir is filling up, to prevent that intestinal contents may escape from the reservoir unintentionally.

From the foregoing it becomes clear that the constriction type pump is adapted to act on said intestinal wall from the outside of the reservoir so as to empty the reservoir by squeezing the reservoir. This can be achieved either hydraulically or mechanically, i.e. by means of a hydraulic type pump or a mechanic type pump.

### Hydraulic Type Pump

According to a preferred embodiment, a hydraulic type pump comprises an electrically driven hydraulic pump, a hydraulically acting member for acting on the intestinal wall of the intestinal reservoir from the outside thereof, and an artificial reservoir, wherein the electrically driven hydraulic pump is adapted to pump hydraulic fluid from the artificial reservoir to the hydraulically acting member. The hydraulically acting member may be tube-like or bag-like to accommodate the reservoir therein. This facilitates implantation and ensures proper placement of the hydraulically acting member relative to the intestinal reservoir over long time.

Preferably, the hydraulically acting member comprises a plurality of hydraulic chambers, each chamber acting on a different section of the intestinal reservoir. By filling the chambers in a predetermined sequence, emptying of the reservoir can be controlled.

In a relatively simple structure, each hydraulic chamber is hydraulically interconnected with two other hydraulic chambers, except the first and last chambers which are hydraulically connected to only one other hydraulic chamber and to the artificial reservoir. Thus, fluid can flow from the artificial reservoir sequentially through the hydraulic chambers and back into the artificial reservoir. Preferably, the hydraulic chambers are interconnected by holes acting as throttles for the fluid. This way, fluid will slowly through the chambers, thereby filling the first chambers before the last chambers, so that intestinal contents in the intestinal reservoir are slowly squeezed out of the intestinal reservoir.

However, more sophisticated structures may be used, e.g. involving one or more actively controlled valves between interconnected chambers instead of the passively acting throttles. Also, instead of interconnecting each hydraulic chamber with respective other two of the hydraulic chambers, one or all of the hydraulic chambers may be arranged either such that they are hydraulically isolated from the respective other chambers and connected only to the hydraulic pump via individual hydraulic control lines, or such that they can be hydraulically isolated from the respective other chambers by individually controlling respective valves within the hydraulic path.

The electrically driven hydraulic pump is preferably adapted to evacuate the hydraulically acting member by applying negative pressure, once the intestinal reservoir has been emptied. New filling of the reservoir with intestinal contents is thereby facilitated.

Alternatively, instead of using negative pressure, one can also rely on passive evacuation of the hydraulically acting member. That is, as the intestinal reservoir fills with intestinal contents again, the fluid within the hydraulically acting member surrounding the intestinal reservoir is automatically urged back into the reservoir. This process can be advantageously supported by means of one or more valves between the hydraulically acting member and the artificial reservoir, which, when in an appropriate operational position, allows fluid to passively flow from the hydraulically acting member back into the artificial reservoir when the reservoir fills with intestinal contents and which, when in an appropriate other position, prevents the fluid to flow from the hydraulically acting member back into the artificial reservoir when the intestinal reservoir is being emptied.

### Mechanical Type Pump

According to another preferred embodiment, a mechanical type pump comprises at least one mechanically acting member for acting on the intestinal wall from the outside of the intestinal reservoir and an electrical motor adapted to drive the mechanically acting member for emptying the intestinal reservoir.

A preferred structure for the mechanically acting member comprises at least one roller adapted to be rolled over the intestinal reservoir for emptying the reservoir from the outside thereof. For instance, two rollers may act simultaneously on opposite outer surface sides of the intestinal reservoir so as to squeeze the reservoir. Alternatively, one roller may act on one outer surface side of the intestinal reservoir against a counteracting plate arranged on an opposite side of the reservoir. For each roller, two tracks may be provided, one on each lateral side of the intestinal reservoir, for guiding the roller or rollers when driven by the electrical motor. Thus, the length of the rollers must be sufficient to bridge the width of the intestinal reservoir. Therefore, similar to the length of the electrical stimulation devices mentioned before, the rollers should have a length of about 10 cm or more and the tracks should preferably also have a length of about 10 cm or more. Preferably, the tracks each have a bent end portion so arranged that it directs away from the reservoir, when implanted. When the roller or rollers are positioned at the track's bent end portion, the intestinal reservoir is not constricted and, thus, intestinal contents can freely enter the intestinal reservoir.

### Valve as Part of the Flow Control Device

As mentioned earlier, in addition to the at least one pump, the flow control device is advantageously provided further with one or more valves for controlling flow to and/or from the reservoir. These valves are preferably implanted inside the patient's body outside a section of the patient's intestine and may comprise at least one element adapted to act on the intestine section from the outside thereof so as to act on and, in particular, prevent intestinal contents flow through the intestine section. This valve arrangement is advantageous inasmuch its installment does not require any surgery on the respective part of the intestine.

For instance, an exit valve should be provided downstream of the intestinal reservoir preventing intestinal contents to flow from the reservoir in its closed position. Preferably, the exit valve is a normally closed valve so that no energy is needed to keep the valve closed during the system's inactive periods.

In addition, an entry valve may be provided allowing intestinal contents to flow towards the intestinal reservoir in its open position. This can be advantageous particularly during the emptying of the reservoir in order to prevent back flow, i.e. when the entry valve is closed. Therefore, the entry valve is preferably a normally open valve. Accordingly, the exit valve and the entry valve are preferably adapted to cooperate such that when one of the two valves is closed, the respective other valve is open, and vice versa.

### Exit Valve

The exit valve may comprise a hydraulic or mechanical constriction device for constricting the intestine section so as to keep the intestine section closed. For instance, a hydraulic constriction device may comprise a compartment with a variable volume adapted to open and close the valve by changing the compartment's volume. The compartment preferably has at least one flexible wall defining an opening for the intestine section to pass through, the opening being adapted to close upon increase of the compartment's volume.

Alternatively, or in addition to the hydraulic or mechanical constriction device, the exit valve may comprise at least one electrical stimulation device adapted to electrically stimulate muscle or neural tissue of the intestine section so as to cause at least partial contraction of the intestine section in order to prevent intestinal contents flow through the intestine section. Similar to the stimulation devices of the electrical stimulation type pump described before, the stimulation device of the exit valve may comprise at least one electrode adapted to apply electric pulses to the intestine section. It is particularly advantageous to make use of an electrical stimulation device which is adapted to stimulate different portions of the intestine section over time. Thus, different portions of the intestine section can be constricted by stimulation at different times in any predetermined stimulation pattern, thereby giving the intestine portions currently not stimulated time to recover and, thus, improving the blood circulation in the respective intestine section.

Furthermore, the electrical stimulation device can specifically be adapted to stimulate, over time, the different portions of the intestine section in a wave like manner in a direction opposite to natural intestinal contents flow. As a result, the valve counteracts the natural intestinal contents flow, thereby improving the valve's closing function.

In particular, where the exit valve comprises a constriction device and an electrical stimulation device in combination, the stimulation device and the constriction device can act on the same intestine section in order to keep the intestine section closed. In this case, the constriction device is preferably adapted to only partly constrict the intestine section in the valve's normal (closed) condition, whereas the stimulation device is adapted to stimulate, over time, different portions of the intestine section in a wave like manner in a direction opposite to natural intestinal contents flow so as to urge out-flowing intestinal contents back towards the intestinal reservoir. This is a very gentle way of preventing intestinal contents from exiting the intestinal reservoir.

In addition, the electrical stimulation device is preferably adapted such that it can likewise be used to pump intestinal contents along the intestine section in a direction of natural intestinal contents flow by, over time, stimulating different portions of the intestine section in a wave-like manner. Emptying of the intestinal reservoir can be supported in this manner. During this process, the partial constriction of the intestine section caused by the valve's hydraulic or mechanical constriction device is released.

### Entry Valve

The entry valve may be simpler in construction than the exit valve, as the entry valve is preferably a normally open valve. Thus, there is only little danger that the tissue of the intestine section could be damaged due to an unduly long time period of interrupted blood flow. Therefore, the entry valve to be implanted upstream from the reservoir in order to control flow of intestinal contents into the reservoir may substantially consist of a hydraulic or mechanical constriction device for constricting said intestine section at the time when the intestinal reservoir is being emptied.

### Motor

It was already mentioned before, that the pump may comprise a motor for automatically driving the pump. The motor is preferably arranged to be driven by electric or electromagnetic energy. The same or a different motor can be arranged for driving the valve or valves, respectively, between the closed and open positions. A motor in the sense of the present disclosure is a device that transforms energy other than mechanical energy into mechanical energy.

Preferably, the motor comprises a servo drive. The effect of the servo drive is that a motor with relatively little power and, thus, a relatively small motor can be used, while the time needed to perform the work increases proportionally. However, since the time for emptying the intestinal reservoir is not very critical, this trade off can be accepted.

A manually operable actuator, e.g. a switch, may be provided for activating the pump or the at least one motor, respectively, from outside the patient's body. The switch is preferably arranged for subcutaneous implantation so as to be easily operable from outside the patient's body.

### Energy Supply

An energy source may be provided for supplying energy directly or indirectly to at least one energy consuming part of the system, in particular for driving the pump. Preferably, the energy source includes a battery or an accumulator, such as one or more of a rechargeable battery and a capacitor, as an energy storage means. The energy storage means is advantageously adapted for being implanted inside the patient's body.

Energy is preferably transmitted wirelessly. Thus, where the energy source is provided for supplying energy directly or indirectly to at least one energy consuming part of the system, the energy source may comprise a wireless energy transmitter adapted to wirelessly transmit energy from outside the patient's body to the at least one energy consuming part. Alternatively, where the energy source includes a battery or an accumulator, in particular one which is implanted in the patient's body, the energy source may comprise a wireless energy transmitter adapted to wirelessly transmit energy from outside the patient's body to the energy storage means.

### Control Unit

It is advantageous to provide a control unit adapted to directly or indirectly control one or more elements of the system, such as for controlling not only the actuation of the pump but also the opening of the exit valve and/or closing of the entry valve, in particular in a manner such that when one of the two valves is closed, the respective other valve is open, and vice versa. The control unit is preferably operable by the patient, e.g. particularly in order to empty the reservoir, such as by actuating the afore-mentioned subcutaneously implantable switch.

Also, the control unit may comprise a first part adapted for implantation in the patient's body and a second part adapted to cooperate with the first part from outside the patient's body. In this case, the control unit can be adapted to transmit data from the external second part of the control unit to the implanted first part of the control unit in the same manner as energy is transmitted to the at least one energy consuming part.

### DA: Implantable Infusion Devices (Basic Aspects)

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating implantable infusion devices, examples of which will now be described.

Thus, the following disclosure relates to an implantable infusion device as well as to a drug delivery system which comprises both the implantable infusion device and at least one extracorporeal component for cooperation from outside a patient's body with the implanted infusion device. The infusion device is particularly suitable for long term applications, i.e. for applications where the patient receives drugs by infusion at predetermined time intervals over months or years. This is typically the case with cytostatica treatment during chemo therapy, insulin treatment in case of diabetes, and the like. Thus, the implantable infusion device is configured to stay at the implantation site for long term use.

The infusion device comprises an infusion needle and a drive unit coupled to the infusion needle and arranged for advancing the tip end of the infusion needle to penetrate any fibrosis when the device is implanted in the patient's body. At least the infusion needle and the drive unit are designed for implantation in the patient's body. Other components such as an energy source, a control unit, a data processing device and/or even a reservoir and/or pump for the drug may be extracorporeal to complete the drug delivery system. However, it is preferred that the aforementioned components be implanted as well and, therefore, belong to the implantable infusion device, most preferably forming an integral part with the remaining components of the implantable infusion device so as to be implantable as a unitary piece.

The infusion device may rest at the implantation site over a long time either for single use or for multiple use. For instance, where it is likely that a patient will suffer an allergic attack, such as heavy allergic reactions affecting e.g. the respiratory tract, in the near future or possibly only within a year or two, the infusion device may be implanted in the patient's body for single use at the appropriate time. Over time, fibrosis will grow on the infusion device. However, at the time of use, the infusion needle may be advanced by the drive unit so as to penetrate any fibrosis, thereby allowing immediate drug delivery through the tip end of the infusion needle into the patient's body. Where the infusion device is implanted adjacent a blood vessel, the tip end of the infusion needle may be advanced into the blood vessel without any risk of formation of thrombosis prior to use.

Where the infusion device is implanted for multiple use, the drive unit is preferably configured for advancing and retracting the tip end of the infusion needle. Thus, each time when the drug is delivered to the patient, the infusion needle will be advanced, the drug injected and the infusion needle retracted again.

Preferably, the infusion needle is disposed within a body of the infusion device with the tip end of the infusion needle being arranged for passing through an outer wall of the body. This prevents any fibrosis from growing into the infusion needle, in which case the infusion needle would still be blocked even after penetrating any fibrosis that has built up in front of the needle.

While it is imaginable that the outer wall be opened for allowing the infusion needle to pass therethrough, it is preferred to arrange the needle for penetrating the outer wall. For that purpose, the outer wall may be made at least partly from a self-sealing material in respect of penetrations resulting from the infusion needle. While the entire body may be made from the self-sealing material, it is advantageous for stability reasons if the self-sealing material forms at least one window area in the outer wall, the window area being positioned for penetration by the tip end of the infusion needle. The window area may be formed by a self-sealing penetration membrane which is preferably integrated in the outer wall by press fitting it into the outer wall.

Typically, the self-sealing material would be made from a polymer material which preferably comprises silicon. Other biocompatible polymer materials may be employed as well.

The self-sealing material may also be a composite material. A particularly preferred embodiment of such composite material comprises at least one outer shape-giving layer and a self-sealing soft material contained within the outer layer. Thus, the outer layer forms a shell for the soft material.

The outer layer may be made from a biocompatible polymer, such as one of those polymers mentioned above, and the self-sealing soft material may be a gel.

While the drive unit of the infusion device may be separate from the body housing the infusion needle, it is preferred to also dispose the drive unit entirely within the body of the infusion device so that the two components can be implanted as a single module.

According to one particularly preferred aspect, the tip end of the infusion needle is laterally movable so as to vary the injection site. For instance, when the infusion device is implanted in a patient's body, it may be placed adjacent a blood vessel after free-dissecting the blood vessel. As set out above, frequent penetration of the same portion of the blood vessel would cause irritation and after a while penetration would become difficult or even impossible. Alternatively letting the infusion needle rest in place within the blood vessel would cause thrombophlebitis followed by thrombosis. Variation of the injection site by laterally displacing the needle at appropriate times may overcome such problems.

For this purpose, the drive unit may comprise a moveable carriage on which the infusion needle is mounted for lateral displacement of the tip end of the infusion needle. The movable carriage may e. g. comprise a turntable and/or a shuttle bus, such as in the form of a slide.

Preferably, the drive unit is configured so as to laterally displace the tip end of the infusion needle each time when said tip end is advanced and/or retracted. Thus, lateral displacement and advancement/retraction of the tip end of the infusion needle are coordinated. The lateral displacement of the tip end of the infusion needle may take place before and/or after an injection.

The mechanism may be such that after a certain number of lateral displacements or after lateral displacement over a predefined distance, the tip end of the infusion needle is laterally returned to its initial position so that the next number of infusions will take place again at locations that have previously been penetrated by the needle. This is particularly suitable where the tip end of the infusion needle is positioned for the penetration of a limited window area or where the infusion device is implanted adjacent a blood vessel.

The infusion needle of the infusion device preferably has a tube-like body closed at the tip end and having a laterally arranged delivery exit port for the delivery of drugs into the patient's body. Therefore, the needle will not cut out any material but will simply divide it during penetration. Thus, when the needle penetrates any material, such as fibrosis and/or the self-sealing penetration membrane, there will be no material entering and blocking the drug delivery passageway.

A reservoir is provided to be coupled to the infusion needle. Typically an infusion liquid will be contained in the reservoir. The reservoir may be arranged separate from the body of the implantable infusion device either for remote implantation within the patient's body or outside the patient's body.

In the latter case, the external reservoir may be connected to the implanted infusion device via a stationary conduit. Refilling an external reservoir is generally easier than refilling an implanted reservoir and, therefore, this is advantageous where a substantial amount of drugs will have to be administered to the patient.

At least a section of a periphery of the reservoir may be made from a flexible material permitting volume changes of the reservoir by deformation of the flexible material as infusion liquid is filled into or drawn out of the reservoir. Thus, the reservoir may be of balloon type. The flexible material may comprise a polymer membrane. A bellow construction is preferable having pre-bent crises to reduce long term degradation.

According to a particular embodiment, drawing liquid from the reservoir may cause a pressure decrease in at least part of the reservoir so that a negative pressure is attained as compared to the pressure in front of the infusion needle. For instance, the reservoir may comprise a gas chamber and a liquid chamber, said chambers being separated by a membrane, e. g. the polymer membrane. When liquid is drawn from the liquid chamber, the pressure in the gas chamber will decrease accordingly.

The reservoir may have an injection port for injecting liquid from outside the human body into the implanted reservoir. That way, the reservoir implanted in the patient's body along with the infusion device may be kept small since the reservoir can be refilled easily at appropriate time intervals.

Preferably, the injection port comprises a self-sealing material in respect of penetrations caused by a replenishing syringe that would be typically used to refill the reservoir through the patient's skin. It is preferable to implant the infusion device, or at least the self-sealing injection port of the reservoir, subcutaneously in the patient's body so that it is easily accessible for refill by means of the syringe.

Where the reservoir forms part of the body of the infusion device, at least a section of a periphery of the reservoir, such as the self-sealing injection port and/or the volume change permitting flexible material, may at least partially constitute the outer wall of the body of the infusion device.

While the reservoir may be compressed automatically or preferably manually in order to inject drugs through the needle into the patient's body, it is preferred to couple a pump between the reservoir and the infusion needle for pumping the drugs from the reservoir to the infusion needle. By means of the pump, it is easy to measure out an exact dose of the drug.

While the type of pump is not critical in the case of extracorporeal use thereof in connection with an external reservoir, one specific type of pump is particularly preferred when the pump is implanted along with the implantable infusion device. More particularly, an implantable pump preferably comprises a valve device having a first and a second valve member, each of the said first and second valve members having a smooth service facing each other so as to form a sealing contact between the first and second valve members and further having different liquid channels that can be brought into alignment by displacement of the two smooth surfaces relative to one another while maintaining the sealing contact. This type of pump is described in great detail in WO 2004/012806 A1 referred to above. The first and second valve members are preferably made from a ceramic material for its excellent sealing capabilities over a long period of time and its inertness to many substances.

The pump may be a membrane type pump, as also described in WO 2004/012806 A1, but is not restricted to this type of pump. The membrane type pump may comprise a membrane displaceable by a piston as the piston moves, the piston being coupled to the valve device so as to slidably displace the first and second valve members relative to one another as the piston moves. Where the infusion needle is disposed within a body of the infusion device, the pump may also be contained in the body of the infusion device or it may be separate from the body of the infusion device for remote implantation within the patient's body.

The pump and/or the drive unit for advancement, retraction and possibly for lateral displacement of the tip end of the infusion needle may be actuated manually. This is particularly practical where the pump is provided extracorporeally separate from the implanted infusion device. Where the pump is implanted along with the infusion device, it may be actuated by mechanical remote control or, more preferably, by a pressure sensitive switch arranged so as to be manually operable when implanted subcutaneously in the patient's body.

Preferably, manual actuation of either the pump or the drive unit simultaneously causes actuation of the other, i.e. the drive unit or the pump. For instance, the pressure built up by the pump may cause the drive unit to advance the infusion needle and when the infusion liquid has been delivered through the tip end of the needle into the patient's body, the pressure relief in the pump will allow a return spring or other resilient means to retract the infusion needle. A mechanical stepper may cause the infusion needle to be laterally displaced upon each advancement and/or retraction.

Instead of manual actuation of the pump and/or drive unit, at least one motor may be provided. The motor may be arranged e. g. for electrically, magnetically or electromagnetically actuating the pump and/or drive unit or for hydraulically actuating the pump and/or drive unit. Preferably, the motor is arranged for actuating either the pump or the drive unit, thereby causing simultaneous actuation of the other, i.e. the drive unit or the pump. A motor may also be provided for actuation of any other energy consuming part of the infusion device.

The term “motor” in the sense of the present disclosure includes anything that employs energy other than manual power and either automatically transforms such energy into kinetic or hydraulic or another type of energy or directly uses such energy to activate the pump, drive unit and/or other part of the infusion device and drug delivery system. As such, it is possible that part of the drive unit also forms a part of the motor, e.g. in the case of an electromagnetically actuated drive unit.

Where the motor forms part of the infusion device and, therefore, is implanted within the patient's body along with the infusion device either separate from the body of the infusion device for remote implantation within the patient's body or contained in the body of the infusion device, coupling elements may be provided either for conductive or for wireless energy transfer from outside the device to the motor. For instance, the motor may be arranged for being wirelessly driven by an external electromagnetic field.

An external energy source for use outside the patient's body, such as a primary energy source or a battery, in particular a rechargeable battery, that may be mounted on the patient's skin may be used to provide energy to the pump and/or drive unit and/or any other energy consuming part of the infusion device. The energy source may in particular be connected to the at least one motor for actuating these components. An external energy source for wireless energy transfer may be adapted to create an external field, such as an electromagnetic field, magnetic field or electrical field, or create a wave signal, such as an electromagnetic wave or sound wave signal.

Where the energy is wirelessly transferred to the implanted infusion device, a transforming device for transforming the wirelessly transferred energy into electric energy may be provided. Such transforming device is preferably adapted to be placed directly under the patient's skin so as to minimize the distance and the amount of tissue between the transforming device and the energy supply means outside the patient's body.

An energy transmission device for wireless energy transfer from the energy source and/or energy storage means to the transforming device may be adapted to generate an electromagnetic field.

Alternatively or in addition, the energy transmission device for wireless energy transfer may be adapted to generate a magnetic field. Also, the energy transmission device for wireless energy transfer may be adapted to generate an electrical field. The wireless energy may also be transmitted by the energy transmission device by at least one wave signal. Such signal may comprise an electromagnetic wave signal, including at least one of an infrared light signal, a visible light signal, an ultraviolet light signal, a laser signal, a microwave signal, a radio wave signal, an X-ray radiation signal and a γ-radiation signal. Also, the wave signal may comprise a sound or ultrasound wave signal. Furthermore, the wireless energy may be transmitted as a digital or analog signal or a combination thereof.

Instead of or in addition to an external energy source, the implantable infusion device may itself be provided with an energy source. Such energy source may be part of or may be contained within the body of the infusion device. However, it may also be provided separate from the body of the infusion device for remote implantation within the patient's body.

Such implantable energy source preferably comprises energy storage means, such as a long-life battery or, more preferably, an accumulator. The accumulator has the advantage of being rechargeable. Preferably, the accumulator comprises a rechargeable battery and/or a capacitor.

Again, coupling elements for conductive or wireless energy transfer from a primary energy source outside the device to the accumulator may be provided for charging the accumulator from outside the patient's body when the device is implanted in the patient's body. Similarly, the accumulator may comprise coupling elements for conductive and/or wireless energy supply to the at least one motor of the infusion device.

While the at least one motor may be provided with actuating means for manual activation of the motor, it is preferred to provide a control unit for controlling the at least one motor. The control unit may also be used to control the pump, drive unit and/or any other energy consuming part of the infusion device and, where the device includes an internal or external energy source, may even be used to control such energy source. The control unit may be adjusted to the patient's individual needs, such that the appropriate amount of medicine will be administered at appropriate time intervals. Automatic administration will substantially relieve the patient.

Preferably, the control unit has a data transfer port for data transfer between an external data processing device outside the patient's body and the control unit implanted in the patient's body, regardless of whether the control unit is contained in the body of the infusion device or whether it is implanted within the patient's body remote from the body of the infusion device. Said data transfer port allows for supervising the control unit to adapt the infusion device to changing needs of the patient. Preferably, the data transfer port is a wireless transfer port for the data transfer, so as to provide easy data exchange between the control unit and the data processing device, e. g. during a visit at the doctor's. Most preferably, the control unit is programmable to further increase its adaptation flexibility.

The control unit—with or without the data transfer port—may also be provided extracorporeally, e. g. mounted on the patient's skin. An external control unit has the advantage of being easily accessible in case of any failure. It is preferably adapted for wireless remote control of the at least one motor implanted with the infusion device.

A control signal transmission device may be provided for wireless transmission of an extracorporeal control signal to an implanted motor. Similarly, a data transmission interface for wirelessly transmitting data from outside the patient's body to a control unit implanted inside the patient's body may be provided. Again, the wireless control signal and/or data transmission may comprise one of the aforementioned wave signals, being digital or analog or a combination thereof.

More preferably, the control signal may be transmitted in the same manner as the energy is transmitted to the motor. For instance, the control signal may be transmitted by modulation of the energy signal, the energy signal thereby serving as a carrier wave signal for the digital or analog control signal. More particularly, the control signal may be a frequency, phase and/or amplitude modulated signal.

Apart from or as a part of the control unit, feedback may be provided on parameters relevant for the treatment of the patient. Such parameters may be either physical parameters of the patient and/or process parameters of the device. For that purpose, at least one feedback sensor is provided for detecting such parameters. For instance, the feedback sensor may be adapted to detect one or more parameters related to any of the following: kind of blood cells, drug level, glucose level, oxygen level, ph level, flow volume in blood vessel, pressure, electrical parameters, distension, distance etc.

The feedback sensors may be connected to the control unit and the control unit may comprise a control program for controlling drug delivery in response to one or more signals of the feedback sensors. In addition or alternatively, feedback data may be transferred from the control unit to the external data processing device. Such feedback data may be useful for the doctor's diagnosis.

The infusion device, as discussed above, may be implanted in the patient's body at various locations. For instance, implantation of the infusion device—or a part thereof—in the patient's abdomen or thorax might be the proper choice where the infusion device or, e. g., its reservoir is relatively voluminous. In that case, one might argue that it may be preferable to implant the infusion device with a completely filled reservoir as it might be difficult to refill the reservoir in the abdomen. However, a subcutaneously positioned injection port connected via a tube to the reservoir may be suitable in this case.

Alternatively, as discussed above, the infusion device may be implanted subcutaneously. Subcutaneous implantation increases the possibilities of wireless energy and/or data transfer between the infusion device and an extracorporeal component of the drug delivery system. Also, refilling the reservoir through an injection port by means of a replenishing needle penetrating through the patient's skin is substantially facilitated when the infusion device is implanted subcutaneously. By means of the replenishing needle, the reservoir may be filled with a volume of infusion liquid of a predetermined dose. It should be understood, however, that depending upon the circumstances any part of the infusion device may be placed in the abdomen or thorax and other parts subcutaneously.

Depending on the individual treatment, it may be advantageous to implant the infusion device within fat tissue or intramuscularly or adjacent a blood vessel or the gastro-intestinal or urinary tract, such as the patient's kidneys, so that the infusion liquid will be injected into the tissue, the muscles or directly into the blood vessel, gastro-intestinal tract or urinary tract. The advantages that may be obtained by a proper choice of location of the infusion device are various and may include better resorption of the drugs when delivered directly so that the drugs will act more quickly and/or may be delivered at a higher dose.

### DB: Implantable Infusion Device (Specific Aspects/Stimulation of Penis Erection)

A system including an implantable control unit/controller for controlling A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can also be used for controlling, communicating with and/or operating implantable infusion devices for the infusion of drugs which will now be described.

The essence of the present disclosure relating to the implantable infusion device provides a drug delivery device for injecting a drug into the patient's body. The device is particularly suitable for, but not limited to, the stimulation of penis erection because it is fully implantable and may be used for delivering a drug in relation to the patient's penis. This will greatly improve the patient's comfort as he no longer needs to pierce himself with the infusion needle, which for many people is not an easy task. More specifically, the drug may be supplied, more specifically injected, into any portion of the patient's body, specifically into both the right and left corpus cavernosum and/or the two deep arteries thereof and/or possibly muscle tissue regulating blood flow through the right and left corpus cavernosum and/or tissue in close proximity to the left and right corpus cavernosum. According to a first, most simple embodiment, the drug delivery device comprises a catheter adapted to be implanted outside the corpora cavernosa in close proximity thereto so as to supply the drugs through the catheter. Alternatively, the catheter may be adapted to be implanted in at least one of the corpora cavernosa so as to supply the drugs directly into the corpus cavernosum through the catheter. The latter alternative is more complicated to implant, but more efficient in use.

According to a preferred, somewhat more complex embodiment, the drug delivery device comprises at least one infusion needle for injecting the drugs, wherein the infusion needle is permanently implanted at an appropriate location inside the patient's body. Due to the permanent implantation of the infusion needle, the injection will always occur at the proper location, said location being selected such that the drug is most effective. While there are many conceivable technical variations for injecting the drug through the infusion needle into the patient's body, such injections are definitely more convenient for the patient once the infusion needle has been implanted as compared to the alternative of injecting the drug into the penis manually from outside the penis. In particular, the infusion needle may be integrated in the above-mentioned catheter.

The infusion needle of the drug delivery device may be adapted to be placed outside the corpora cavernosa in close proximity thereto and may further be adapted to change its position outside the corpora cavernosa, when the drug delivery device is implanted. Thus, whenever the needle's position is changed outside the corpora cavernosa, drugs are delivered through the infusion needle into a region outside the corpora cavernosa. Alternatively, the at least one infusion needle being placed outside the corpora cavernosa may be adapted to be advanced into and retracted from the at least one of the corpora cavernosa. In this case, whenever the needle is advanced into the corpus cavernosum, the drugs are delivered through the infusion needle into the corpus cavernosum. This latter alternative is more efficient in use.

Most preferably, one or more infusion needles are arranged at least with the tip end or ends thereof disposed within (at least one) housing so as to penetrate the housing's outer wall or walls in at least one penetration area, more preferably in two or more different penetration areas. Arranging the infusion needle's tip end in a housing prevents any fibrosis from growing into the infusion needle. Preferably, the needle or needles are fully accommodated in said housing. Where more than one needle is provided, the needles—or at least the tip ends thereof—may be arranged in separate housings. The housing or housings are adapted for implantation inside the patient's body adjacent the two corpora cavernosa and/or the two deep arteries thereof and/or adjacent muscle tissue regulating blood flow through the patient's left and right corpus cavernosum and/or adjacent tissue in close proximity to the two corpora cavernosa. Where the at least one infusion needle is arranged for penetrating the at least one housing in two or more different penetration areas, the distance between the different penetration areas to be penetrated by the at least one infusion needle is selected such that the respective parts of the patient's body are pierced whenever the drug is to be injected. As will be described below, two or more infusion needles may be provided in a single housing or in different housings in order to inject the drug in the two or more different penetration areas, or a single infusion needle may be provided in a single housing along with an appropriate drive unit for displacing the tip end of the infusion needle so as to penetrate the single housing's outer wall in the respective different penetration areas.

Furthermore, according to the present disclosure, at least one drive unit, which is also adapted for implantation inside the patient's body, is coupled to the at least one infusion needle so as to—at least—advance and retract the tip end of the at least one infusion needle in such a way that it penetrates at least two of said different penetration areas within the housing's or housings' outer wall, so as to allow for stimulation of penis erection by injecting the substance through said at least two different penetration areas via the at least one infusion needle. For instance, the at least one infusion needle may be arranged for penetrating the at least two different penetration areas either simultaneously (e.g. where a plurality of needles, i.e. at least two needles, are provided) or in immediate time succession (e.g. where a single needle is provided).

Preferably, a single command or single action from the patient is sufficient for injecting the substance through the at least two penetration areas, either due to a corresponding mechanical structure of the drive unit or due to a suitably configured control unit controlling the drive unit. This will make the handling of the system easy for the patient.

Where two different penetration areas are pierced in immediate time succession, the time delay between the penetration of the first and the second of the penetration areas is preferably as short as possible, more preferably less than 120 seconds, and most preferably less than 60 seconds. This can be achieved by means of a properly controlled drive unit. A longer time delay would be inconvenient for the patient. Therefore, it is preferred that, once the infusion needle has been retracted from a first of the two penetration areas, it is immediately advanced to the second of the penetration areas.

While it is possible according to one aspect of the present disclosure to actively open the outer wall or walls of the at least one housing for allowing the infusion needle to penetrate the outer wall, it is preferred according to another aspect of the present disclosure to arrange the needle so as to penetrate the outer wall by piercing through the outer wall. For that purpose, the outer wall may either comprise flaps to be pushed aside by the infusion needle as the infusion needle is advanced, or the outer wall may be made at least in the penetration areas from a material which is self-sealing in respect of penetrations resulting from the at least one infusion needle. While the entire housing may be made from the self-sealing material, it is advantageous for stability reasons if the self-sealing material forms at least one window area in the outer wall, the window area being positioned for penetration by the tip end of the at least one infusion needle. The window area may be formed by a self-sealing penetration membrane which is preferably integrated in the outer wall by press fitting it into the outer wall.

Typically, the self-sealing material would be made from a polymer material which preferably comprises silicone. Other biocompatible polymer materials, such as polyurethane and the like, may be employed as well.

The self-sealing material may also be a composite material. A particularly preferred embodiment of such composite material comprises at least one outer shape-giving layer and a self-sealing soft material contained within the outer layer. Thus, the outer layer forms a shell for the soft material.

The outer layer may be made from a biocompatible polymer, such as one of those polymers mentioned above, and preferably the self-sealing soft material may be a gel.

Instead of a self-sealing material, the part of the outer wall to be penetrated by the infusion needle may comprise one or more flaps in the penetration areas through which the infusion needle or needles can pass. This can reduce the force needed for the infusion needle to penetrate the outer wall, as compared to the penetration of a self-sealing membrane. The flap is preferably arranged to be pushed aside by the infusion needle upon advancement of the infusion needle.

Alternatively, the outer wall may comprise at least one door in the penetration areas. A drive is connected to the door for actively opening the door so as to allow for the infusion needle to be advanced through the opened door. Again, the door may comprise a flap, such as a resilient, normally closed flap. It is particularly preferred if the drive connected to the door forms part of the drive unit coupled to the infusion needle. More specifically, the arrangement may be such that advancement of the infusion needle by means of the drive unit simultaneously causes the drive to open the door.

Where a single housing is provided for the at least one infusion needle (or at least for the tip ends thereof) or where two or more penetration areas are arranged in a single housing, the penetration areas may be arranged in the housing so that they can be placed either adjacent to both the right and left corpus cavernosum of the patient's penis and/or the two deep arteries of the right and left corpus cavernosum and/or adjacent to muscle tissue regulating blood flow through the right and left corpus cavernosum and/or in sufficiently close proximity to another type of tissue allowing both the first and second corpus cavernosum to become turgid when the particular drug is injected thereinto.

The at least one infusion needle preferably has a tube-like body closed at the tip end and provided with a laterally arranged delivery exit port for delivery of the drug into the particular body part. Therefore, the needle will not cut out any material but will simply divide it during penetration. Thus, when the needle penetrates any material, such as fibrosis and/or the self-sealing penetration membrane, there will be no material entering and blocking the drug delivery passageway.

As mentioned above, a separate infusion needle may be provided for each of the two or more penetration areas. Thus, where injection is desired to occur in only two different areas to provoke penis erection, two separate infusion needles may be advanced through the corresponding penetration area of the respective housing—preferably simultaneously—and retracted again after injection.

According to a preferred embodiment of the present disclosure, a plurality of two or more infusion needles are provided for each of the different penetration areas and arranged for penetrating different penetration sites within each of said different penetration areas. This allows for penetrating different sites within each penetration area at different times, thereby giving the human tissue time to recover from the piercing by the infusion needle. This can be achieved with a drive unit suitably configured to advance and retract one infusion needle in each of said different penetration areas at one time, and to advance and retract a different infusion needle in each of said different penetration areas at a different time.

The system may further comprise at least one reservoir adapted for implantation inside the patient's body, the reservoir being in fluid connection with the at least one infusion needle so as to supply to the infusion needle the substance to be injected into the patient's body. Also, at least one pump, which is also adapted for implantation inside the patient's body, may be provided to advance the substance from the reservoir to the at least one infusion needle.

Since it is preferred for reasons of space constraints to implant the reservoir remote from the injection areas, it can be advantageous to employ long infusion needles that are flexibly bendable. The tip ends of such infusion needles would then be arranged within at least one first housing so as to penetrate the outer wall thereof upon advancement of the long infusion needle. The at least one first housing accommodating the tip end of such long infusion needle or needles can be arranged in close proximity to the injection area so that the tip end can penetrate the outer wall of the first housing upon advancement of the long infusion needle. The respective other end of the infusion needle or needles, i.e. the infusion needle's rear end, is arranged in at least one second housing which can be implanted inside the patient's body remote from the first housing in an area with less space constraints. The injection needle would be sufficiently long to bridge the distance from the second housing for remote implantation to the first housing and further through the first housing up to the outer wall of the first housing to be penetrated by the needle. The long and flexibly bendable infusion needle may be guided within a suitable sheath. Since the rear end of the long infusion needle or needles is remotely implanted within the patient's body, other components of the system cooperating with rear end of the long infusion needle may also be implanted remote from the injection area, such as the reservoir for storing the substance to be injected, the pump for advancing the drug from the reservoir to the infusion needle and further through the needle into the patient's body, and a motor for actuating the system's active parts. In a particularly preferred embodiment, the tip end of the long infusion needle is advanced by advancing the entire needle from the infusion needle's rear end. In this context, it is particularly advantageous to also arrange at least a part of the drive unit, preferably the entire drive unit, for advancing and retracting the tip end of the infusion needle remote from the injection area, preferably within the second housing and even more preferably in a common housing with the remotely implanted reservoir. More preferably, most or all of the active parts, such as a motor, pump and the like, may be accommodated in the remotely implanted second housing, whereas the first housing only includes passive elements.

A drive unit according to the present disclosure includes not only the drive itself, such as an electric motor, but also those components which are involved in transforming the driving energy provided by the drive into movement of the at least one needle, such as transmission gears and the like.

For instance, in the case of the long flexibly bendable infusion needle, the drive unit may be such that the infusion needle is advanced and/or retracted by turning the infusion needle or by turning an element cooperating with the infusion needle. More specifically, the drive unit for advancing and retracting the infusion needle may comprise a screw drive connection. For instance, the drive of the drive unit may turn a screw threadingly engaged with a rack coupled to the infusion needle so that rotation of the screw will cause the infusion needle to be advanced or retracted. The screw and rack of the screw drive connection are preferably accommodated in the remotely implanted second housing but may also be arranged in the housing accommodating the tip end of the needle. Instead of the screw, the infusion needle itself may be rotated by means of a suitable drive so that threading on the needle engaging a fixedly mounted rack causes the infusion needle to advance or retract upon rotation of the infusion needle. Between the first and second housings, the infusion needle is preferably guided in a sheath, so as to reduce friction and prevent growth of fibrosis that might hinder movement of the needle.

According to a particularly preferred aspect of the present disclosure, the tip end or ends of the at least one infusion needle are laterally movable, so as to vary the penetration sites within a particular penetration area of the at least one housing's outer wall, thereby varying the injection site within the particular injection area in the patient's body. As set out above, frequent piercing of the same body part may cause irritation, eventually making further piercing difficult or even impossible. Variation of the injection site by laterally displacing the needle upon each injection cycle may overcome such problems. Accordingly, the at least one drive unit in the common housing is preferably configured to laterally displace the tip end or ends of the at least one infusion needle to different penetration sites within a penetration area. More specifically, where two or more infusion needles are provided for penetrating two or more different penetration areas, the drive unit is preferably configured to laterally displace the tip ends of the infusion needles simultaneously. This can be achieved e.g. by jointly mounting the tip ends of the infusion needles on a movable carriage of the drive unit, such as a turntable and/or a shuttle, possibly in the form of a slide. Thus, the drive unit for advancing and retracting the tip end or ends of the at least one infusion needle is preferably configured so as to also laterally displace a tip end each time the tip end is advanced or retracted.

Thus, the lateral displacement and the advancement/retraction of the tip end of an infusion needle is coordinated. The lateral displacement of the tip end of the infusion needle may take place before and/or after an injection. The mechanism may be such that after a certain number of lateral displacements or after lateral displacement over a predefined distance, the tip end of the infusion needle is laterally returned to its initial position so that the next number of infusions will take place again at locations that have previously been penetrated by the needle. It is even preferred to configure the drive unit such that the tip end of the infusion needle is displaced in at least two different lateral directions within one penetration area. For instance, when the infusion needle has laterally returned to its initial position, the next number of infusions may take place somewhat laterally offset above or below the first number of penetration sites. This permits a two-dimensional array of penetration sites to be obtained. In particular, the drive unit may be configured to displace the tip end of the infusion needle along a curved path, which will result in a three-dimensional array of penetration sites if the needle is displaceable in different lateral directions.

Where two or more infusion needles are provided for penetrating two or more different penetration areas, the infusion needles or, in the case of the afore-mentioned long and flexibly bendable infusion needles, at least the tip ends thereof may be contained in a common housing in spaced apart relationship, with the drive unit being configured to advance and retract the tip ends of the infusion needles so as to penetrate the outer wall of the common housing in said at least two different penetration areas, again preferably simultaneously. The infusion needles—or at least the tip ends thereof—may be arranged one above the other within the common housing. Generally speaking, it is preferable in such a situation that the direction of lateral displacement of the tip ends of the infusion needle within different penetration areas is different from, in particular perpendicular to, the direction of distance between the different penetration areas. Alternatively, where the infusion needles are arranged with great lateral distance between each other, the direction of lateral displacement of the tip ends of the infusion needles within each of two different penetration areas may generally be the same as the direction of distance between the two different penetration areas. Placing the two or more injection needles—or at least the tip ends thereof—in a common housing simplifies the procedure of fixing the needles in place close to the injection areas. Furthermore, a single drive unit may be used for advancing and retracting the tip ends of the plurality of infusion needles, this making the entire system less voluminous. The use of a single drive unit is particularly advantageous where a major part of the drive unit is also contained in the common housing, i.e. where a major part of the drive unit is also to be implanted close to the very constrained injection area.

Instead of providing two or more infusion needles for penetrating two or more different penetration areas, it is likewise possible to provide a single infusion needle or the tip end of a single needle within the housing and to implant the housing within the patient's body adjacent the two or more injection areas. In this case, the drive unit may be configured so as to laterally displace the tip end of the single one infusion needle between various lateral positions such that the infusion needle can penetrate the housing's outer wall in the different penetration areas. The distance of lateral displacement of the single infusion needle between the different penetration areas would amount to 3 mm, 4 mm, 5 mm or even more upon each successive injection. Such successive injections are preferably in immediate time succession, preferably not exceeding 120 seconds between two injections, more preferably not exceeding 60 seconds. Most preferably, the drive unit will be adapted to initiate advancement of the one infusion needle to a second one of the plurality of penetration areas once it has been retracted from a first one of the penetration areas.

An implantable infusion device comprising a single, laterally displaceable infusion needle contained within a housing so as to penetrate the housing's outer wall at different penetration sites is generally known from WO 2007/051563. However, this prior art infusion device is not aimed at being used for the stimulation of penis erection. Also the prior art device is neither intended nor configured for injecting drugs simultaneously or quasi-simultaneously in immediate time succession in two or more different injection areas. The drive unit of the prior art device is instead configured to administer the drug at a different penetration site of a single injection area at each time of operation. For instance, the prior art device may be placed along a blood vessel so as to inject drugs at different injection sites within a single injection area of the blood vessel. Thus, the distance of lateral displacement of the tip end of the infusion needle between one injection and a next following injection is not configured in the prior art device such that different injection areas within the patient's body could be reached.

Turning back to the present disclosure, it is again preferable, when the patient desires to achieve another penis erection at a later point of time, that the single infusion needle does not penetrate the same penetration site within the particular penetration area of the housing's outer wall, but that the drive unit is configured to laterally displace the tip end of the one infusion needle to different penetration sites within each of the different penetration areas. Again, the direction of lateral displacement of the tip end of the one infusion needle within each of the different penetration areas may either be the same as the direction of lateral displacement of the tip end of the infusion needle between the different penetration areas, or may be different from, in particular perpendicular to, the direction of lateral displacement of the tip end of the infusion needle between the different penetration areas. Depending upon the particular configuration of the system, this may be achieved with a single, multifunctional drive unit or with a plurality of different drive units suitably arranged to work in coordinated fashion. Even a combination of these alternatives is possible. For instance, when after a number of infusion cycles the single infusion needle has laterally returned to its initial position, the next number of infusions within the same penetration area may take place somewhat laterally offset above or below the first number of penetration sites. Thus, a two-dimensional array of penetration sites can be obtained in each penetration area, thereby keeping the maximum dimensions of the penetration areas at a minimum.

Where the housing or at least the window area thereof is formed spherically, even a three-dimensional array of penetration sites through the housing's outer wall can be obtained by means of a suitably adapted drive unit for the needle displacement. This greatly increases the system's flexibility of use.

Regardless of the number of needles involved and regardless of the particular penetration site array to be achieved, it is preferable to configure the drive unit such that the lateral displacement of the tip end of the infusion needle or needles is achieved automatically during advancement and/or retraction of the tip end of the needle or needles. For instance, where the infusion needle is mounted on a movable carriage for the lateral displacement of the tip end of the needle, such as on a turntable or a shuttle, e.g. in the form of a slide, the drive unit may comprise a stepper which is adapted to automatically advance the movable carriage a predefined distance upon each advancement and/or retraction of the infusion needle.

Now, turning to the reservoir, it should be considered that long term storage is not possible with many currently available drugs, this being particularly true of drugs stimulating penis erection. Where long term storage is desired, the drug to be injected would typically be provided as a first substance and mixed with a second substance for injection shortly before the injection is performed. Therefore, according to a preferred embodiment of the present disclosure, the reservoir of the system comprises at least one first compartment, e.g. for accommodating an infusion liquid such as a saline solution, and at least one second compartment, e.g. containing a drug, in particular a drug in dry form, for mixing with the infusion liquid of the first compartment. The drug may be in powder form and, more specifically, may be a freeze-dried drug. In particular, the drug contained in the second compartment would be a drug for stimulating penis erection. A mixing chamber may be provided for mixing the substance from the first compartment with the substance from one or more of the at least one second compartment.

The number of the second compartments may be huge, such as 50 or more, in particular 100 or more. This would not constitute a particular problem in terms of space constraints since the amount of drugs required for each stimulation of penis erection is extremely little and would amount to a few micrograms. Furthermore, the reservoir may be adapted for implantation within the patient's body remote from the housing containing the needle, such as close to the symphyseal bone. There is a lot of space available above the patient's symphyseal bone, and the drugs could be delivered to the tip end of the needle through an appropriate conduit. If desired, one can inject pure saline solution after the drug injection has been completed so as to clean the conduit and needle from any drug residue. Such cleaning injection could be done through a different penetration area of the housing's outer wall into tissue of the patient which would not affect penis stimulation.

Preferably, the second compartments containing the drug are liquid-tightly sealed against the first compartment, with a mechanism being provided for individually opening a connection between the second compartments and the first compartment.

According to a preferred embodiment, the second compartments are mounted in a plate so as to open towards a first side of the plate and the opening mechanism is adapted to act on the second compartments from a second side of the plate opposite the first side of the plate so that the compartments open to the first side of the plate. Thus, the second compartments may be pushed from their rear side (second side of the plate) so as to open frontward into e.g. a mixing chamber in which the content of the opened second compartments mixes with the content of the first compartment of the reservoir, such as with saline solution. More specifically, the second compartments may be mounted in the plate as displaceable drug containers and the opening mechanism may be adapted to displace the drug containers such that they deliver their drug contents in the manner described.

Alternatively, the plate may be rotatable so as to allow the drug containers to be brought into alignment with a conduit upon rotation of the plate. Thus, when the drug is brought into alignment with such conduit, it may be mixed with e.g. saline solution pumped through the conduit towards the infusion needle.

According to another preferred embodiment, the second compartments are mounted on a tape wound up on a reel. A plurality of rows of the second compartments may be arranged on the tape in side-by-side relationship in a direction different to the winding direction of the tape. This way, the length of the tape can be reduced. It is particularly preferable if the tape is contained in a replaceable cassette. Thus, when all of the second compartments of the tape are emptied, the tape can be easily replaced by replacing the cassette.

As mentioned above, while the reservoir may generally be part of the housing accommodating the at least one infusion needle, it is preferred to arrange the reservoir separate from the housing for remote implantation within the patient's body.

At least a section of a periphery of the first compartment of the reservoir may be made from a flexible material permitting volume changes of the first compartment by deformation of the flexible material as infusion liquid is filled into or drawn out of the reservoir. Thus, the reservoir may be of balloon type. The flexible material may comprise a polymer membrane. A bellows construction is preferable having pre-bent creases to reduce long term degradation.

According to a particular embodiment, drawing liquid from the reservoir may cause a pressure decrease in at least part of the reservoir so that a negative pressure is attained as compared to the pressure in front of the infusion needle. For instance, the first compartment of the reservoir may comprise a gas chamber and a liquid chamber, said chambers being separated by a membrane, e.g. the polymer membrane. When liquid is drawn from the liquid chamber, the pressure in the gas chamber will decrease accordingly.

The reservoir may have an injection port for injecting liquid from outside the human body into the implanted reservoir. That way, the reservoir implanted in the patient's body along with the drug delivery device may be kept relatively small since the reservoir can be refilled easily at appropriate time intervals, possibly with a doctor's aid.

Preferably, the injection port comprises a self-sealing material in respect of penetrations caused by a replenishing syringe that would be typically used to refill the reservoir through the patient's skin. It is preferable to implant the self-sealing injection port of the reservoir subcutaneously in the patient's body so that it is easily accessible for refill by means of the syringe.

The conduit or conduits for connecting the remotely implanted reservoir with the infusion needle or needles should have a length sufficient to bridge the distance between the patient's symphyseal bone and the inferior fascia of the patient's urogenital diaphragm, where the housing is preferably to be placed. Accordingly, the conduit should have a length of 10 cm or more.

While it has already been pointed out that drugs, in particular the drugs for stimulating penis erection, may degrade upon long term storage, another important influence on drug degradation is the storage temperature. Some drugs have to be stored in a refrigerator at low or at least moderate temperature. A preferred embodiment of the present disclosure therefore provides for a cooling device for keeping the content within at least one compartment of the reservoir at a temperature below 37° C. This can be achieved with relatively little energy supply if the amount of drugs to be cooled is extremely little, as explained above, and if furthermore the drug compartment within the reservoir is thermally insulated. For instance, the reservoir may be comprised in an insulation chamber.

It is preferred to provide the cooling device with a heat exchanger for exchanging with the patient's body heat generated by the cooling device. Such heat exchanger may be implanted within the patient's body remote from the cooling device to safely dissipate the heat energy in an area where it cannot adversely affect the content of the reservoir.

The cooling device can be of a variety of different types. According to a first embodiment, the cooling device may contain at least two different chemicals reacting with each other, thereby consuming thermal energy which energy is drawn from the contents within the reservoir so that a cooling effect on the contents is achieved. The two chemicals may be provided in separate chambers and a flow control device may be provided to bring together certain amounts of the two different chemicals so as to control the amount of thermal energy drawn from the contents within the reservoir.

According to a second embodiment, the cooling device may comprise at least one Peltier element. A Peltier element is an electrothermal converter causing a temperature difference to occur when an electric current is flowing through the element, based on the Peltier effect. While one part of the Peltier element cools down, a different part thereof heats up. Such heat may again be removed by means of a heat exchanger or simply by providing the particular part generating the heat with an enlarged surface so that the heat is directly dissipated into the adjacent body part of the patient.

According to a third embodiment, the cooling device may be of a refrigerator-type construction. That is, heat exchanging pipes within a chamber to be cooled and heat exchanging pipes outside the chamber for dissipating the heat energy absorbed in the cooling chamber are provided along with a compressor for compressing the refrigerant gas when it exits the cooling chamber and an expansion valve for expanding the refrigerant gas before it re-enters the cooling chamber.

Turning now to the pump for advancing the infusion liquid from the reservoir to the infusion needle or needles, such pump may be a manually driven pump or an automatically driven pump. The manually driven pump may be formed from a balloon which may be manually compressed if suitably arranged under the patient's skin. The balloon type pump may at the same time serve as a reservoir for the infusion liquid, in particular for the saline solution. Preferably, however, an automatically driven pump is used. While the type of pump is not critical, one specific type of pump is particularly preferred. More particularly, an implantable pump preferably comprises a valve device having a first and a second valve member, each having a smooth surface facing each other so as to form a sealing contact between the first and second valve members and further having different liquid channels that can be brought into alignment by displacement of the two smooth surfaces relative to one another while maintaining the sealing contact. This type of pump is described in great detail in WO 2004/012806 A1. The first and second valve members are preferably made from a ceramic material for its excellent sealing capabilities over a long period of time and its inertness to many substances.

The pump may be a membrane type pump, as also described in WO 2004/012806 A1, but is not restricted to this type of pump. The membrane type pump may comprise a membrane displaceable by a piston as the piston moves, the piston being coupled to the valve device so as to slidably displace the first and second valve members relative to one another as the piston moves. Preferably, the pump will be implanted separate from the housing accommodating the needle or needles for remote implantation within the patient's body.

Due to the space constraints within the patient's body in the area where injection is to take place, it is advantageous to implant as many components of the system as possible remote from the housing accommodating the infusion needle or needles. In this context, the drive unit may comprise a mechanical drive element for transmitting kinetic energy from a remote location within the patient's body to the at least one infusion needle. The mechanical drive element may comprise a rotating shaft by which a considerable distance can be bridged within the patient's body. The rotating shaft may, upon rotation about its axis of rotation, cause movement of the infusion needle either directly or indirectly. More specifically, the rotating shaft may be in the form of a worm screw which, when turned, causes the infusion needle or needles to advance and retract and/or causes the infusion needle or needles to move laterally upon each advancement/retraction. Individual rotating shafts or worm screws may be provided for each individual infusion needle and/or for advancing and retracting the tip end of the infusion needle or needles on the one hand and laterally displacing the tip end of the infusion needle or needles on the other hand. Most preferably, the rotating shaft or worm screw is flexibly bendable, so that it can be freely arranged within the patient's body.

Alternatively or in addition, the drive unit may comprise at least one wire directly or indirectly cooperating with the infusion needle so as to cause movement of the infusion needle upon actuation of the wire. Thus, the wire may be pulled at one end thereof which is located within the patient's body remote from the injection sites. Preferably, the wire extends through the same conduit which connects the infusion needle or needles with the reservoir. More specifically, pulling the wire may cause the tip end of the infusion needle or needles to displace laterally from a first to a second of the different penetration areas or from a first penetration site to a second penetration site within a single one of the different penetration areas. A single pulling wire may be sufficient to cause movement of the infusion needle in one direction, whereas a spring element or any other pre-tensioning means may be provided to urge the infusion needle back to the initial starting position or to a different starting position. Alternatively, two pulling wires may be provided to move the infusion needle back and forth in a single dimension.

According to a preferred embodiment, the infusion needle is arranged for two-dimensional lateral displacement. This can be achieved by means of two pulling wires, preferably cooperating again with spring elements or other pre-tensioning means to provide a counterforce to be overcome by pulling the wires. Alternatively, three pulling wires may be provided to laterally displace the tip end of the infusion needle back and forth along at least two directions within a two-dimensional plane.

A pulling wire may also be arranged to advance or retract the infusion needle by pulling the wire. Again, a spring element or other pre-tensioning means may be provided to urge the infusion needle back to its initial starting position or to a different starting position.

Alternatively, the drive unit may comprise a hydraulic drive for transmitting hydraulic energy from a remote location within the patient's body to the at least one infusion needle for advancing the tip end thereof and/or for laterally displacing the tip end thereof. The infusion liquid itself may be used as the hydraulic medium providing the hydraulic energy, or a secondary liquid different from the infusion liquid may be used.

Further alternatively, the drive unit may comprise one or more electric motors inside the housing accommodating the at least one infusion needle. In this case, energy may be transmitted from a remote location within the patient's body to the at least one motor by means of appropriate wiring. Again, as in the two afore-described alternatives, a single motor may be provided for advancing and retracting the tip end of the infusion needle or needles and for laterally displacing the tip end of the infusion needle or needles, or individual motors may be provided for each individual infusion needle and/or for advancing the tip ends of the infusion needle or needles on the one hand and laterally displacing the infusion needle or needles on the other hand.

Even further alternatively, the drive unit may comprise an electromagnetic drive for laterally displacing and/or for advancing and retracting the tip end of the infusion needle or needles. For instance, the electromagnetic drive may comprise a group of electromagnets composed of a plurality of laterally spaced apart electromagnet first parts and at least one electromagnet second part, the electromagnet second part cooperating with an energized one of the electromagnet first parts. The electromagnet second part is fixedly connected to the infusion needle or needles either directly or indirectly so that upon energization of one or more of the electromagnet first parts the electromagnet second part and, thus, the infusion needle or needles will be caused to move. The arrangement of the electromagnet first parts and second part may be such that the electromagnet first parts are arranged in a first plane and the electromagnet second part is movable in front of or behind the first plane. Alternatively, the electromagnet first parts may face each other, thereby defining a first plane between them, and the electromagnet second part may be movable within the first plane. Depending on which one or ones of the electromagnet first parts are energized, the electromagnet second part with the infusion needle or needles fixed thereto will move accordingly. The electromagnet first parts preferably each include a magnetic coil.

In either one of the aforementioned alternatives, it is advantageous to transmit the driving energy through the conduit that connects the at least one infusion needle with the remotely implanted reservoir. That is, in the case of a mechanical drive element in the form of a wire or rotating shaft, the wire/shaft and the infusion liquid may be guided through a common conduit. The common conduit may comprise two separate paths, one for the shaft or wire and one for the infusion liquid. Such a common conduit facilitates the handling and arrangement of the system during implantation. Similarly, the wiring for transmitting electric energy to the motor or to the electromagnetic drive may be guided through a conduit connecting the infusion needle or needles with the reservoir.

Where the pump and/or drive unit is not actuated manually, a drive in the form of a motor may be arranged e.g. for electrically, magnetically or electromagnetically actuating the pump and/or drive unit or for hydraulically actuating the pump and/or drive unit. The motor is preferably arranged for actuating either the pump or the drive unit, thereby causing simultaneous actuation of the other, e.g. the drive unit or the pump. A motor may also be provided for actuation of any other energy consuming part of the drug delivery device. More specifically, a plurality of motors may be provided, e.g. an individual motor for each infusion needle and/or an individual motor for displacing the tip end of the infusion needle in a lateral direction on the one hand and for advancing the tip end of the infusion needle through the housing's outer wall on the other hand.

Again, for reasons of space constraints in the area of implantation of the housing accommodating the infusion needle or needles, it is advantageous to remotely implant the motor within the patient's body separate from the housing. Again, actuating means may be provided for manual activation of the motor or motors, such actuating means preferably being adapted for subcutaneous implantation.

The term “motor” according to the present disclosure includes anything that employs energy other than manual power and either automatically transforms such energy into kinetic or hydraulic or another type of energy or directly uses such energy to activate the pump, drive unit and/or other part of the overall system. As such, it is possible that part of the drive unit also forms part of the motor, e.g. in the case of an electromagnetically actuated drive unit.

Coupling elements may be provided either for conductive or for wireless energy transfer from outside the patient's body to the motor. For instance, the motor may be arranged for being wirelessly driven by an external electromagnetic field.

An energy source for providing energy to at least one of the pump, the drive unit and the drive (motor) for driving the drive unit, and any other energy consuming part of the system may be provided. For instance, an external energy source for use outside the patient's body, such as a primary energy source or a battery, in particular a rechargeable battery, that may be mounted on the patient's skin, may be used to provide energy to the pump and/or drive unit and/or any other energy consuming part of the system. The energy source may in particular be connected to the at least one motor for actuating these components. An external energy source for wireless energy transfer may be adapted to create an external field, such as an electromagnetic field, magnetic field or electrical field, or create a wave signal, such as an electromagnetic wave or sound wave signal.

Where the energy is wirelessly transferred to the implanted components, a transforming device for transforming the wirelessly transferred energy into electric energy may be provided. Such transforming device is preferably adapted to be placed directly under the patient's skin so as to minimize the distance and the amount of tissue between the transforming device and the energy supply means outside the patient's body.

Instead of or in addition to an external energy source, the system may comprise an internal power supply for implantation within a patient's body. While such implantable power supply may be part of or may be contained within the housing accommodating the infusion needle or needles, it is preferred to provide the implantable energy source separate from the housing for remote implantation within the patient's body. Such implantable power supply preferably comprises energy storage means for long term storage of energy, such as a long-life battery or, more preferably, an accumulator. The accumulator has the advantage of being rechargeable. Preferably, the accumulator comprises a rechargeable battery and/or a capacitor.

Again, coupling elements for conductive or wireless energy transfer from an external power supply outside the patient's body to the accumulator may be provided for charging the accumulator from outside the patient's body when the device is implanted in the patient's body. Similarly, the accumulator may comprise coupling elements for conductive and/or wireless energy supply to the at least one motor of the drug delivery device.

A feedback subsystem, which may be part of a control unit described below, can advantageously be provided to send feedback information relating to the energy to be stored in the energy storage means from inside the human body to the outside thereof. The feedback information signal may be sent wirelessly. The feedback information is used for adjusting the amount of energy supply, in particular the amount of wireless energy transmitted by the energy transmitter. The feedback information preferably includes information on a parameter related to the charging process for controlling the charging process. It may specifically relate to an energy balance which is defined as the balance between an amount of wireless energy received inside the human body and an amount of energy consumed by the at least one energy consuming part. Alternatively, the parameter may relate to an energy balance which is defined as the balance between a rate of wireless energy received inside the human body and a rate of energy consumed by the at least one energy consuming part. The consumed energy preferably includes the energy that is consumed by the process of charging the implantable power supply.

Preferably, a control unit is provided for controlling an amount of infusion liquid to be administered through the at least one injection needle. A single command from the patient to the control unit, such as a single actuation of a press button or other type of switch, is sufficient for causing the control unit to control the injection of the drugs at two different locations within the patient's body. The control unit may be provided for controlling at least one of the pump, the drive unit and the motor and any other energy consuming part of the system and, where the system includes an internal or external energy source, said energy source. Again, the control unit is preferably separate from the housing accommodating the infusion needle or needles so as to be implantable within the patient's body. The control unit may be adjusted such that the appropriate amount of drugs will be administered at the appropriate time to the particular one of the injection sites. Automatic administration will substantially relieve the patient.

Preferably, the control unit has a data transfer port for data transfer between an external data processing device outside the patient's body and the control unit implanted in the patient's body, regardless of whether the control unit is contained in the housing accommodating the infusion needle or needles or whether it is implanted within the patient's body remote from said housing. The data transfer port allows for monitoring the control unit to adapt the system to changing needs of the patient. Preferably, the data transfer port is a wireless transfer port for the data transfer, so as to provide easy data exchange between the control unit and the external data processing device, e.g. during a visit to the doctor. Most preferably, the control unit is programmable to further increase its adaption flexibility. Instead of or in addition to the external data processing device, the control unit may comprise an external component for manual operation by the patient for setting into operation the control unit.

Apart from or as a part of the control unit, feedback may be provided on parameters relevant for the treatment. Such parameters may be either physical parameters of the patient and/or process parameters of the system. For this purpose, at least one feedback sensor is provided for detecting such parameters. For instance, the feedback sensor may be adapted to detect one or more parameters relating to any of the following: drug level, flow volume in blood vessel, pressure, electrical parameters, distension, distance, etc.

The feedback sensors may be connected to the control unit and the control unit may comprise a control program for controlling drug delivery in response to one or more signals from the feedback sensors. In addition or alternatively, feedback data may be transferred from the control unit to the external data processing device. Such feedback data may be useful for the doctor's diagnosis.

The penetration areas of the wall or walls of the housing or housings within which the infusion needle or needles are disposed may be arranged in the patient's body at various locations. For instance, they may be arranged adjacent the left and right corpora cavernosa and/or the two deep arteries running through the left and right corpora cavernosa and/or muscle tissue regulating blood flow through the patient's left and right corpora cavernosa and/or another kind of tissue in close proximity to the left and right corpora cavernosa.

A holder may be used to secure the corpora cavernosa to the housing or housings so that the housing rests in place.

Other components of the system are preferably remotely implanted, such as adjacent the patient's symphyseal bone. As discussed above, some components of the system may be implanted subcutaneously. Subcutaneous implantation increases the possibilities of wireless energy and/or data transfer between the implanted and the extracorporal parts of the system. Also, refilling the reservoir through an injection port by means of a replenishing needle penetrating through the patient's skin is substantially facilitated when an injection port of the reservoir is implanted subcutaneously. In particular, the compartment of the reservoir containing the saline solution might need to be refilled frequently, whereas the other compartments comprising individual small doses of the drug would need no refill. It should be understood, however, that depending upon the circumstances any implantable component of the system may be placed in the abdomen or even in the thorax.

Activating means for direct manual operation by the patient may also be provided to be implanted subcutaneously, e.g. for setting into operation one or more of the aforementioned motors or for simply setting into operation the control unit of the system. Such activating means may be in the form of a subcutaneously implantable switch manually operable by the patient from outside the patient's body.

The various aforementioned features of the present disclosure may be combined in any way if such combination is not clearly contradictory. The present disclosure will now be described in more detail in respect of preferred embodiments and with reference to the accompanying drawings. Again, individual features of the various embodiments may be combined or exchanged unless such combination or exchange is clearly contradictory to the overall function of the device.

### Long, Flexibly Bendable Infusion Needles

In particular, while most of the implantable infusion device embodiments addressed above relate to systems having one or more infusion needles entirely accommodated within one or more housings, it is likewise possible in all of these embodiments to employ long, flexibly bendable needles having only the front ends thereof accommodated in said housing or housings and having the respective rear ends disposed in one or more second housings remotely implanted within the patient's body. Embodiments comprising long, flexibly bendable infusion needles will be addressed hereinafter.

Accordingly, the at least one implantable infusion needle is long and flexibly bendable, with the respective tip end or ends being disposed within (at least one) first housing so as to penetrate the first housing's outer wall or walls in at least one penetration area, preferably in two or more different penetration areas. As will be described below, two or more infusion needles may be provided in order to inject the drug in the two or more different penetration areas, or a single infusion needle may be provided along with an appropriate drive unit for displacing the tip end of the infusion needle so as to penetrate the first housing's outer wall in the respective different penetration areas. Arranging the infusion needle's tip end in said first housing prevents any fibrosis from growing into the infusion needle.

The use of one or more long flexible needles is chosen for reasons of space constraints in the area where the injection of the drugs has to occur. The at least one first housing accommodating the tip end of such long infusion needle or needles can be kept small and, therefore, can be arranged conveniently in close proximity to a constrained injection area, where the tip end can penetrate the outer wall of the first housing upon advancement of the long infusion needle. The respective other end of the long, flexibly bendable infusion needle or needles, i.e. the infusion needle's rear end, is arranged in a second housing which can be implanted inside the patient's body remote from the first housing in an area with fewer space constraints. The injection needle is sufficiently long to bridge the distance from the second housing for remote implantation to the first housing and further through the first housing up to the outer wall of the first housing to be penetrated by the needle. Thus, since the respective other end of the infusion needle or needles is remotely implanted within the patient's body, other components of the system cooperating with the rear end of the infusion needle may also be implanted remote from the injection area, such as a reservoir for storing the substance to be injected, a pump for advancing the substance from the reservoir to the infusion needle and further through the needle into the patient's body, a motor for actuating the system's active parts, at least one drive unit for—at least—advancing (and possibly retracting) the infusion needle or needles in such a way that it penetrates at least one penetration area—preferably two or more different penetration areas—so as to allow for injecting the substance through said penetration area or areas via the at least one infusion needle, and so forth. In a particularly preferred embodiment, the tip end of the infusion needle is advanced by advancing the entire needle from the infusion needle's rear end. It is particularly advantageous to arrange at least a part of the drive unit, preferably the entire drive unit, for advancing the infusion needle within the second housing. More preferably, a reservoir and/or most or all of the active parts, such as a motor, a pump and the like, may also be implanted remotely within the patient's body and, more preferably, may be accommodated in the remotely implanted second housing, whereas the first housing only includes passive elements, possibly including passive elements of the drive unit. In this context it is to be understood that a drive unit according to the present invention includes not only the drive itself, such as an electric motor, but also those components which are involved in transforming the driving energy provided by the drive into movement of the at least one needle, such as transmission gears and the like. Other components such as an energy source, a control unit and/or a data processing device may be extracorporal to complete the system. However, it is preferred that the aforementioned components be implanted as well, preferably forming an integral part with the remaining components of the system so as to be implantable as a unitary piece with the second housing.

### E: Lubrication of a Synovial Joint

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control of an implantable medical device for lubrication of a synovial joint. Examples of such devices for lubrication of joints will now be described.

An improved technique for lubricating a damaged or worn out joint of a human or mammal patient that, on the one hand, sufficiently lubricates the joint and, on the other hand, has a minimal infection risk, is herein presented.

The implantable lubrication device at least comprises, firstly, a reservoir that stores a lubricating fluid and, secondly, a fluid connection that introduces the stored lubricating fluid into the damaged joint when the lubrication device is implanted in a patient's body. The lubricating device can be completely implanted into the patient's body such that a damaged joint can post-operatively be appropriately lubricated from within the patient's body. This significantly reduces the infection risk for the patient and permits a post-operative supply of lubricating fluid to the damaged joint, continuously, intermittently, periodically or as required, e.g. depending on a fluid level within the joint.

An implanted lubrication system according to the present invention comprises the implanted lubrication device and a lubricating fluid stored in its reservoir which is introduced into the joint by means of its fluid connection.

Further compulsory or optional components of the implantable lubrication device, such as a reservoir, a pump or motor, an energy source, a control unit, may also be fully implemented within a patient's body. Such components may belong to the implantable infusion device or form an integral part of the implantable lubrication system separate from the actual implantable infusion device. Since the implantable lubrication device is entirely implantable in the patient's body, i.e. the implanted lubrication system provides both a functionality for storing and a functionality for transporting lubricating fluid within the patient's body, the complete flow path of the lubricating fluid for lubricating the joint lies within the patient's body. Hence, there is no longer a need for extracorporeal injections into the joint.

The fluid connection comprises a fluid connection device that connects the reservoir of the implantable infusion device to the joint, thus establishing a flow path for the lubricating fluid from the reservoir into the joint. The fluid connection device is also fully implantable and preferably consists of a flexible tube or the like that is suitable for post-operatively transporting the lubricating fluid stored in the reservoir to the joint.

Further, the fluid connection comprises an infusion member connected to the fluid connection device. The infusion member may be introduced into the patient's body in close relation to or inside the joint during a surgery, such that, post-operatively, the lubricating fluid can be introduced into the joint. It may be arranged to intermittently inject lubricating fluid into the joint, e.g. periodically or if the fluid level falls below a predetermined threshold, e.g. upon actuation by a drive mechanism and dependent on sensor data. Alternatively and preferably, the infusion member may also be arranged to continuously inject lubricating fluid into the joint, e.g. a predetermined amount of lubricating fluid per time unit, e.g. one drop per hour or the like.

An intermittent or periodical injection may be achieved e.g. by an infusion needle that is placed in close relation to the joint during a surgery such that post-operatively it may be intermittently advanced into the joint in the right position and retracted thereafter by a drive mechanism, thereby allowing intermittent lubrication of the joint through a tip end of the infusion needle. The drive mechanism is configured for advancing and retracting the tip end of the infusion needle into and out of the joint. While the drive mechanism may be separate from the infusion needle and/or the fluid connection device, it is nevertheless arranged as an integral part of the implantable lubrication device such that it is entirely implanted into the patient's body.

Alternatively, the infusion member may comprise an infusion tube that is permanently placed in the joint in order to continuously introduce lubricating fluid into the joint. In this case a separate drive mechanism for advancing/retracting an infusion needle is not needed, since the infusion tube may be of a reasonably soft material that does not, or not appreciably, disturb the normal operation of the joint. Therefore, the infusion tube may lie within the joint on a permanent basis, such that lubricating fluid may be continuously inserted into the joint space.

Preferably, the reservoir of the implantable lubrication device comprises a reservoir coupled to the fluid connection device for storing the lubricating fluid. Typically the lubricating liquid is contained in the reservoir. The reservoir may be arranged as a separate part of the implantable lubrication device, which has to be separately implanted in the patient's body. In order to establish a proper fluid flow of lubricating fluid into the joint, the reservoir may be adapted to change its volume for creating an adequate pressure within the fluid connection device and the infusion member to transport the lubricating fluid into the joint.

Therefore, at least a portion of a periphery of the reservoir may comprise a flexible outer wall for changing the volume of the reservoir by deformation of the flexible material as the lubricating fluid is filled into or drawn out of the reservoir and for causing a fluid flow from the reservoir to the joint through the fluid connection device.

Thus, the reservoir may be of balloon type. The flexible material may comprise a polymer membrane. A bellows construction is preferable having pre-bent creases to reduce long term degradation. Drawing liquid from the reservoir into the fluid connection device and to the joint may cause a pressure decrease in at least part of the reservoir so that a negative pressure is attained as compared to the pressure in front of the infusion needle or the infusion tube at the joint end of the fluid connection device. For instance, the reservoir may comprise a gas chamber and a liquid chamber, said chambers being separated by a membrane, e. g. the polymer membrane, and act as a spring for changing the volume of the reservoir, such that the pressure in the gas chamber will decrease when lubricating liquid is drawn from the liquid chamber into the fluid connection device.

The reservoir may also have a refill injection port for refilling lubricating liquid from outside the human body into the implanted reservoir. The reservoir implanted in the patient's body along with the fluid connecting device may thus be kept small since the reservoir can be refilled easily at appropriate time intervals. Preferably, the injection port comprises a self-sealing material in respect of penetrations caused by a replenishing syringe that would be typically used to refill the reservoir through the patient's skin. It is preferable to implant the reservoir of the lubrication device, or at least the self-sealing injection port of the reservoir, subcutaneously in the patient's body so that it is easily accessible for refill by means of the syringe.

While the reservoir may be compressed manually in order to introduce lubricating fluid through the fluid connection device and the infusion needle or infusion tube into the patient's joint, it is preferred to connect a pump to said fluid connection device and couple it between the reservoir and the infusion member for pumping the lubricating fluid from the reservoir into the joint. By means of the pump, it is easy to measure out an exact dose of the lubricating fluid and thereby supply an appropriate amount of lubricating fluid into the joint in a continuous or intermittent way.

The implantable pump preferably comprises a valve device having a first and a second valve member, each of the said first and second valve members having a smooth surface facing each other so as to form a sealing contact between the first and second valve members and further having different liquid channels that can be brought into alignment by displacement of the two smooth surfaces relative to one another while maintaining the sealing contact. This type of pump is described in great detail in WO 2004/012806 A1. The first and second valve members are preferably made from a ceramic material for its excellent sealing capabilities over a long period of time and its inertness to many substances. The pump may be a membrane type pump, as also described in WO 2004/012806 A1, but is not restricted to this type of pump. The membrane type pump may comprise a membrane displaceable by a piston as the piston moves, the piston being coupled to the valve device so as to slidably displace the first and second valve members relative to one another as the piston moves.

Preferably, manual actuation of either the pump or the drive mechanism simultaneously causes actuation of the other, i.e. the drive mechanism or the pump. For instance, the pressure built up by the pump may cause the drive mechanism to advance the infusion needle and when the infusion liquid has been delivered through the tip end of the needle into the patient's body, the pressure relief in the pump will allow a return spring or other resilient means to retract the infusion needle.

The implanted pump may be actuated by mechanical remote control, by a pressure sensitive switch arranged so as to be manually operable when implanted subcutaneously in the patient's body, or by a sensor mechanism that measures the fluid level in the joint and actuates the pump (and the drive mechanism for advancement and/or retraction of the infusion needle) and actuates the pump if the measured fluid level falls below a predetermined threshold. Preferably, actuation of either the pump or the drive mechanism simultaneously causes actuation of the other, i.e. the drive mechanism or the pump. For instance, the pressure built up by the pump may cause the drive mechanism to advance the infusion needle and when the lubricating liquid has been delivered through the tip end of the infusion needle into the patient's body, the pressure relief in the pump will allow a return spring or other resilient means to retract the infusion needle.

For actuating the pump and, if utilized, a drive mechanism for advancing and/or retracting an infusion needle into/out of the joint, and for directly or indirectly causing a lubricating fluid flow into the joint within said lubrication device, at least one motor may be provided. The motor may be arranged e.g. for electrically, magnetically or electromagnetically actuating the pump and/or drive mechanism or for hydraulically actuating the pump and/or drive mechanism. Preferably, the motor is arranged for actuating either the pump or the drive mechanism, thereby causing simultaneous actuation of the other, i.e. the drive mechanism or the pump. A motor may also be provided for actuation of any other energy consuming part of the infusion device.

The term “motor” in the sense of the present invention includes anything that employs energy other than manual power and either automatically transforms such energy into kinetic or hydraulic or another type of energy or directly uses such energy to activate the pump, drive mechanism and/or other parts of the implanted lubricating device. As such, it is possible that part of the drive mechanism also forms a part of the motor, e.g. in the case of an electromagnetically actuated drive mechanism.

The motor forms part of the lubricating device and is implanted within the patient's body either separate from the body of the lubricating device for remote implantation within the patient's body or contained in the body of the lubrication device. Coupling elements may be provided either for conductive or for wireless energy transfer from outside the device to the motor. For instance, the motor may be arranged for being wirelessly driven by an external electromagnetic field. It is also possible to use an external energy source for use outside the patient's body, such as a primary energy source or a battery, in particular a rechargeable battery, that is mounted on the patient's skin to provide energy to the pump and/or drive mechanism and/or any other energy consuming part of the lubrication device. The energy source may in particular be connected to the at least one motor for actuating these components. An external energy source for wireless energy transfer may be adapted to create an external field, such as an electromagnetic field, magnetic field or electric field, or create a wave signal, such as an electromagnetic wave or sound wave signal.

Where the energy is wirelessly transferred to the implanted lubrication device, a transforming device for transforming the wirelessly transferred energy into electric energy may be provided. Such transforming device is preferably adapted to be placed directly under the patient's skin so as to minimize the distance and the amount of tissue between the transforming device and the energy supply means outside the patient's body.

An energy transmission device for wireless energy transfer from the energy source and/or energy storage means to the transforming device may be adapted to generate an electromagnetic field. Alternatively or in addition, the energy transmission device for wireless energy transfer may be adapted to generate a magnetic field. Also, the energy transmission device for wireless energy transfer may be adapted to generate an electric field. The wireless energy may also be transmitted by the energy transmission device by at least one wave signal. Such signal may comprise an electromagnetic wave signal, including at least one of an infrared light signal, a visible light signal, an ultraviolet light signal, a laser signal, a microwave signal, a radio wave signal, an X-ray radiation signal and a γ-radiation signal. Also, the wave signal may comprise a sound or ultrasound wave signal. Furthermore, the wireless energy may be transmitted as a digital or analog signal or a combination thereof.

Instead of or in addition to an external energy source, the implantable lubrication device may itself be provided with an energy source. Such energy source may be part of or may be contained within the body of the lubricating device. However, it may also be provided separate from the body of the lubricating device for remote implantation within the patient's body.

Such implantable energy source preferably comprises energy storage means, such as a long-life battery or, more preferably, an accumulator. The accumulator has the advantage of being rechargeable. Preferably, the accumulator comprises a rechargeable battery and/or a capacitor.

Again, coupling elements for conductive or wireless energy transfer from a primary energy source outside the device to the accumulator may be provided for charging the accumulator from outside the patient's body when the device is implanted in the patient's body. Similarly, the accumulator may comprise coupling elements for conductive and/or wireless energy supply to the at least one motor of the infusion device.

While the at least one motor may be provided with actuating means for manual activation of the motor, it is preferred to also provide a control unit for controlling the at least one motor. The control unit may also be used to control the pump, drive mechanism and/or any other energy consuming part of the implanted lubricating device and, where the device includes an internal or external energy source, may even be used to control such energy source. The control unit may be adjusted to the patient's individual needs, such that the appropriate amount of medicine will be administered at appropriate time intervals.

Automatic administration will substantially relieve the patient.

Preferably, the control unit has a data transfer port for data transfer between an external data processing device outside the patient's body and the control unit implanted in the patient's body, regardless of whether the control unit is contained in the body of the lubrication device or whether it is implanted within the patient's body remote from the body of the lubricating device. Said data transfer port allows for supervising the control unit to adapt the infusion device to changing needs of the patient. Preferably, the data transfer port is a wireless transfer port for the data transfer, so as to provide easy data exchange between the control unit and the data processing device, e. g. during a visit at the doctor's. Most preferably, the control unit is programmable to further increase its adaptation flexibility.

The control unit—with or without the data transfer port—may also be provided extracorporeally, e. g. mounted on the patient's skin. An external control unit has the advantage of being easily accessible in case of any failure. It is preferably adapted for wireless remote control of the at least one motor implanted with the infusion device.

A control signal transmission device may be provided for wireless transmission of an extracorporeal control signal to an implanted motor. Similarly, a data transmission interface for wirelessly transmitting data from outside the patient's body to a control unit implanted inside the patient's body may be provided. Again, the wireless control signal and/or data transmission may comprise one of the aforementioned wave signals, being digital or analog or a combination thereof. More preferably, the control signal may be transmitted in the same manner as the energy is transmitted to the motor. For instance, the control signal may be transmitted by modulation of the energy signal, the energy signal thereby serving as a carrier wave signal for the digital or analog control signal. More particularly, the control signal may be a frequency, phase and/or amplitude modulated signal.

Apart from or as a part of the control unit, feedback may be provided on parameters relevant for the treatment of the patient. Such parameters may be either physical parameters of the patient and/or process parameters of the device. For that purpose, at least one feedback sensor is provided for detecting such parameters. For instance, the feedback sensor may detect the level of lubricating fluid within the joint or other parameters relating to the condition of the joint and its lubrication. The feedback sensors may be connected to the control unit and the control unit may comprise a control program for controlling delivery of lubricating fluid to the joint in response to one or more signals of the feedback sensors. In addition or alternatively, feedback data may be transferred from the control unit to the external data processing device. Such feedback data may be useful for the doctor's diagnosis.

Preferably, the fluid connection device consists of two fluid connection portions each being connected to the reservoir and having an infusion member at its open end to be inserted into the joint space. The two fluid connection portions may be arranged within the patient's body such that, post-operatively, they form a circular flow path for the lubricating fluid via the joint, i.e. from the pump and/or reservoir to the joint (via a first fluid connection portion) and back to the pump and/or reservoir (via a second fluid connection portion). Under the pressure created by a pump or a flexible outer wall of a reservoir the lubricating fluid may circulate intermittently or continuously through the circular flow path, the second fluid connection portion picking up the lubricating fluid in the joint space that has been inserted thereinto via the first fluid connection portion.

Since due to the circular flow path the lubricating fluid is at least partly re-used after passing the joint, the fluid is soiled in the course of time by impurities or other foreign particles which may reduce the quality and desired effects of the lubricating fluid. The circular fluid connection device of the implantable lubrication device, therefore, may also comprise a filtering device having a filter connected into the circular flow path to remove impure particles from the soiled circulating lubricating fluid. Preferably, the filtering device is adapted to regularly clean the filter and to remove the particles filtered out of the lubricating fluid. These removed impurities or foreign particles may then be deposited into a sealed deposition space or may be given back to the patient's body, e.g. to the surrounding tissue or into a blood vessel or the like.

The lubricating device may be implanted in the patient's body at various locations, preferably as near as possible to the damaged joint to be lubricated. For instance, implantation of the lubrication device—or a part thereof—in the thigh for lubricating the femur ball or knee joint is possible. When the lubricating device or, e.g., its reservoir is relatively voluminous, it may be preferable to implant the lubricating device with a completely filled reservoir as it might be difficult to refill the reservoir in the abdomen. However, a subcutaneously positioned refill injection port connected via a tube to the reservoir may be suitable in this case. Alternatively, the lubrication device may also be implanted subcutaneously. Subcutaneous implantation increases the possibilities of wireless energy and/or data transfer to/from the lubricating device, if desired. Also, refilling the reservoir through a refill injection port by means of a replenishing needle penetrating through the patient's skin is substantially facilitated when the lubricating device is implanted subcutaneously. Depending on the individual treatment, it may be advantageous to implant the lubricating device within fat tissue or intramuscularly or adjacent a joint so that the lubricating fluid can be injected into the particular joint.

Apart from the lubrication device with its various components described above, an implanted lubrication system according to the present invention comprises an appropriate lubricating fluid that is adapted to be stored in the reservoir and to be introduced into the joint by the implanted fluid connection. Preferably, the lubricating fluid is resorbable and bio-compatible in order to ensure resorption of and biological and chemical interaction with the synthetic lubricating fluid by the patient's body in the same way as with a physiological lubricating fluid. Preferably, the lubricating fluid is a hyaluronic acid or the like.

In one embodiment the implantable medical device is adapted to lubricate at least one artificial contacting surface carrying weight in a joint, when implanted in said human or mammal body, said artificial contacting surface replacing at least the surface of at least one of a mammal's joint at least two contacting surfaces, said medical device further comprising, at least one outlet adapted to receive lubricating fluid from said a reservoir, and wherein
said medical device is adapted to be operable by an artificial operation device to distribute lubricated fluid from said reservoir and transport it to said at least one artificial contacting surface.

The implantable medical device may have said reservoir and the joint spaced apart, comprising a conduit for fluid connection between said reservoir and the joint.

The implantable medical device may have the reservoir adapted to be placed subcutaneously or in a cavity in the body in a region of the patient selected from a group of regions consisting of:
a. the abdominal region,
b. the inguinal region,
c. the pelvic region, and
d. the thigh region.

The implantable medical device may thus be placed in the abdomen.

The refill injection port may be adapted to be implanted subcutaneously or in connection with bone.

The implantable medical device may be adapted to lubricate one artificial contacting surface and an opposite contacting surface of the hip or knee joint of a human or mammal patient.

The knee joint having a medial and lateral contacting weight carrying surface, wherein said implantable medical device may be adapted to lubricate said artificial contacting surface on the medial side of the knee joint of a human or mammal patient.

The knee joint having a medial and lateral contacting weight carrying surface, wherein said implantable medical device may be adapted to lubricate said artificial contacting surface of the lateral side on the knee Joint of a human or mammal patient.

A mammal joint having at least two contacting surfaces. The medical device is adapted to lubricate at least one artificial contacting surface which has replaced at least the surface of at least one of the mammal's joint contacting surfaces in said joint. Furthermore the medical device comprises at least one inlet adapted to receive a lubricating fluid from a reservoir.

Normally at least one channel is at least partly integrated in the artificial contacting surface in connection with the at least one inlet for distributing the lubricating fluid to the surface of the artificial contacting surface. The medical device could be adapted to be operable by an operation device to distribute lubricated fluid from a reservoir. The possibility to inject a lubricating fluid intermittently or when needed reduces the friction in the joint and enables an optimal level of lubrication in the joint.

According to one embodiment of the implantable medical device, it could be adapted to distribute the lubricating fluid to the surface of the artificial contacting surface on two or more portions of the artificial contacting surface for lubricating the artificial contacting surface. The distribution in more than one portion could enable a more even distribution of the lubricating fluid.

According to another embodiment the medical device the reservoir adapted to hold the lubricating fluid could be an implantable reservoir placed in a cavity of the body, subcutaneously or in connection with bone.

The implantable medical device could further comprise an operation device adapted to transport a lubricating fluid from said reservoir to the artificial contacting surface for lubricating the artificial contacting surface.

According to one embodiment a reservoir could be adapted to hold the lubricating fluid and the operation device according to any of the embodiments herein could be adapted to transport the lubricating fluid from the reservoir to the artificial contacting surface for lubricating the artificial contacting surface. The operation device could be powered and could comprise a pump adapted to pump fluid from the reservoir to the artificial contacting surface for lubricating the artificial contacting surface.

The operation device, according to any of the embodiments herein could comprise a reservoir, pre-loaded with pressurized lubrication fluid.

According to another embodiment, the implantable medical device could further comprise an implantable injection port adapted to allow, by injection into the injection port, to pre-load the reservoir with pressurized lubricating fluid.

The implantable medical device could, according to one embodiment, further comprise a valve adapted to close the connection between the reservoir and the artificial contacting surface. The reservoir could be adapted to be placed in a unit separate from the artificial contacting surface and adapted to be connected to the artificial contacting surface with a conduit. The reservoir could comprise a moveable wall portion adapted to move and change the volume of the reservoir, the wall portion could be a powered wall portion which could comprise a motor.

According to another embodiment, the implantable medical device could comprise at least one outlet and at least one further channel at least partly integrated in the artificial contacting surface. The medical device could be adapted to allow circulation of a lubricating fluid; out from the artificial contacting surface through the outlet and in to the artificial contacting surface through the inlet. The circling of the fluid could be performed by means of an operation device adapted to circulate the lubricating fluid. The circling system could comprise a reservoir adapted to add fluid to the circulating lubricating fluid, and/or a filter to clean the circulating lubricating fluid.

The operation device according to any of the embodiments could be adapted to intermittently transport a lubricating fluid to the artificial contacting surface.

The implantable medical device could according to one embodiment comprise a sensor adapted to sense a physical parameter inside the joint, or a pressure or volume of the lubricating fluid, or a functional parameter of the operation device to control the operation device to adjust the flow of lubricating fluid to the artificial contacting surface.

The reservoir according to any of the embodiments could be connected to the artificial contacting surface through a conduit. The inlet could comprise a connection part, for connecting the conduit to any part of the medical device. The conduit, according to any of the embodiments could comprise a plurality of portions, which could be adapted to be connected to each other through an inter-connecting part. A first portion of the conduit could be in connection with the medical device, and the second portion of the conduit could be in connection with the reservoir. The conduit could according to one embodiment be adapted to pass through a bone of the body for long-term keeping a passage way open through the bone, allowing the lubricating fluid to reach the artificial contacting surface. According to another embodiment the conduit is adapted to pass through a joint capsule of the body for long-term keeping a passage way open through the joint capsule, allowing the lubricating fluid to reach the artificial contacting surface and according to yet another embodiment the conduit is adapted to pass through the pelvic bone from the opposite said of the acetabulum and into the hip joint.

The implantable medical device could be adapted to lubricate a hip joint of a patient, in which case the artificial contacting surface of the medical device could be adapted to at least partly replace a contacting surface of the Acetabulum, and/or the Caput femur.

The implantable medical device could according to one embodiment lubricate a second artificial contacting surface. According to one embodiment the first artificial contacting surface comprises a convex shape towards a center of the hip joint and the second artificial contacting surface comprises a concave shape towards the center of the hip joint. The first artificial contacting surface is according to this opposite embodiment adapted to be fixated to the pelvic bone of the human patient, and the second artificial contacting surface is adapted to be fixated to the femoral bone of the human patient. The artificial contacting surface could be adapted to be introduced into the hip joint through a hole in the pelvic bone, from the abdominal side of the pelvic bone, an operational method which allows the hip joint capsule to be kept intact.

The reservoir could according to one embodiment be adapted to be placed inside, or at least partly inside of a bone of the patient, the bone could for example be the femoral bone, the pelvic bone or the collum femur of the patient.

According to another embodiment, the reservoir could be adapted to be placed subcutaneously or in a cavity in the body, which could be a cavity in a region selected from a group of regions consisting of: the abdominal region, the inguinal region, the pelvic region, and the thigh region.

The implantable medical device could according to one embodiment comprise an injection port for filling of the reservoir. The injection port could comprise a self-sealing membrane, which for example could be a Parylene coated silicone membrane. The injection port could be adapted to be implanted subcutaneously, in connection with bone or in a cavity of the body.

The reservoir could be adapted to place the lubrication fluid under pressure. For achieving the pressure the reservoir could be adapted to be spring loaded, comprise a chamber adapted to hold a compressed gas or comprise an elastic wall adapted to create the pressure. According to one embodiment the reservoir comprises a Parylene coated silicone elastic wall.

According to another embodiment, the implantable medical device is adapted to lubricate a knee joint of a patient. The artificial contacting surface to be lubricated could according to one embodiment be adapted to at least partly replace a contacting surface of the femoral bone, which could be a contacting surface of the Tibia bone and/or the femoral bone.

According to one embodiment the medical device is adapted to lubricate at least one of the medial or lateral part of the contacting surface of tibia of the knee joint and according to another embodiment the implantable medical device is adapted to lubricate at least one of the medial or lateral part of the contacting surface of the femoral bone of the knee joint. In yet another embodiment the medical device is adapted to lubricate both the contacting surface of the femoral bone of the knee joint and the contacting surface of the tibia bone of the knee joint.

According to one embodiment the reservoir according to any of the embodiments is adapted to be refilled from outside of the human body, the refilling could be performed through an implantable injection port.

According to one embodiment, the reservoir is adapted to hold a pressure, which is possible to increase through injection of a lubricating fluid through the injection port.

The implantable medical device according to any of the embodiments could be adapted to be a part of a system, which further could comprise at least one switch implantable in the patient for manually and non-invasively controlling the implantable medical device. The energized system enables an operation device to operate the lubrication performed by the medical device.

The system could according to one embodiment further comprise a hydraulic device having an implantable hydraulic reservoir, which could be hydraulically connected to the implantable medical device. The implantable medical device could be adapted to be non-invasively regulated by manually pressing the hydraulic reservoir.

According to another embodiment, the system could further comprise a wireless remote control for non-invasively controlling the implantable medical device. The wireless remote control could comprise at least one external signal transmitter and/or receiver, further comprising an internal signal receiver and/or transmitter implantable in the patient for receiving signals transmitted by the external signal transmitter or transmitting signals to the external signal receiver. The wireless remote control could further be adapted to transmit at least one wireless control signal for controlling the implantable medical device. The wireless control signal could comprise a frequency, amplitude, or phase modulated signal or a combination thereof. The wireless remote control could further be adapted to transmit an electromagnetic carrier wave signal for carrying the control signal.

According to another embodiment the system could comprise a wireless energy-transmission device for non-invasively energizing the implantable energy consuming components of the implantable medical device with wireless energy. The wireless energy could comprise a wave signal, selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal, gamma radiation signal, an electric field, a magnetic field, a combined electric and magnetic field.

A control signal in the system could comprise an electric field, a magnetic field, a combined electric and magnetic field. The signal could comprise an analogue signal, a digital signal, or a combination of an analogue and digital signal. For powering the energy consuming components of the implantable medical device, the implantable system could comprise an implantable internal energy source. According to another embodiment the system comprises an external energy source for transferring energy in a wireless mode, wherein the internal energy source is chargeable by the energy transferred in the wireless mode.

According to a further embodiment the system could further comprise a sensor or a measuring device sensing or measuring a functional parameter correlated to the transfer of energy for charging the internal energy source, and a feedback device for sending feedback information from inside the patient's body to the outside thereof, the feedback information could be related to the functional parameter sensed by the sensor or measured by the measuring device.

According to yet another embodiment, the system could further comprise a feedback device for sending feedback information from inside the patient's body to the outside thereof, the feedback information being related to at least one of a physical parameter of the patient and a functional parameter related to the implantable medical device.

The system could according to one embodiment further comprise a sensor and/or a measuring device and an implantable internal control unit for controlling the implantable medical device in response to information being related to at least one of a physical parameter of the patient sensed by the sensor or measured by the measuring device and a functional parameter related to the implantable medical device sensed by the sensor or measured by the measuring device. The physical parameter could according to one embodiment be a pressure or a motility movement.

The system could according to one embodiment comprise an external data communicator and an implantable internal data communicator communicating with the external data communicator, the internal communicator feeds data related to the implantable medical device or the patient to the external data communicator and/or the external data communicator feeds data to the internal data communicator.

The system according to any of the embodiments herein, could further comprise a motor or a pump for operating the implantable medical device, or a hydraulic operation device for operating the implantable medical device. The operation device could comprise a servo designed to decrease the force needed for the operation device to operate the implantable medical device instead the operation device acting a longer way, increasing the time for a determined action.

According to one embodiment the system could further comprise an operation device for operating the implantable medical device. The wireless energy could be used in its wireless state to directly power the operation device to create kinetic energy for the operation of the implantable medical device, as the wireless energy is being transmitted by the energy-transmission device. The system could also comprise an energy-transforming device for transforming the wireless energy transmitted by the energy-transmission device from a first form into a second form energy.

The energy-transforming device could be adapted to directly power implantable energy consuming components of the implantable medical device with the second form energy, as the energy-transforming device transforms the first form energy transmitted by the energy-transmission device into the second form energy. The second form energy could comprise at least one of a direct current, pulsating direct current and an alternating current. The energy of the first or second form could comprise at least one of magnetic energy, kinetic energy, sound energy, chemical energy, radiant energy, electromagnetic energy, photo energy, nuclear energy thermal energy, non-magnetic energy, non-kinetic energy, non-chemical energy, non-sonic energy, non-nuclear energy and non-thermal energy.

For protecting the system or the parts of the system, the system could further comprise an implantable electrical component including at least one voltage level guard and/or at least one constant current guard. A control device could be arranged to control the transmission of wireless energy from the energy-transmission device, and an implantable internal energy receiver for receiving the transmitted wireless energy, the internal energy receiver could be connected to implantable energy consuming components of the implantable medical device for directly or indirectly supplying received energy thereto, the system could further comprise a determination device adapted to determine an energy balance between the energy received by the internal energy receiver and the energy used for the implantable energy consuming components of the implantable medical device, the control device could be adapted to control the transmission of wireless energy from the external energy-transmission device, based on the energy balance determined by the determination device.

The determination device could be adapted to detect a change in the energy balance, the control device could be adapted to control the transmission of wireless energy based on the detected energy balance change. The determination device could in turn be adapted to detect a difference between energy received by the internal energy receiver and energy used for the implantable energy consuming components of the implantable medical device, and the control device could be adapted to control the transmission of wireless energy based on the detected energy difference.

The energy-transmission device could comprise a coil placed externally to the human body, which in turn could further comprise an implantable energy receiver to be placed internally in the human body and an electric circuit connected to power the external coil with electrical pulses to transmit the wireless energy, the electrical pulses having leading and trailing edges, the electric circuit adapted to vary first time intervals between successive leading and trailing edges and/or second time intervals between successive trailing and leading edges of the electrical pulses to vary the power of the transmitted wireless energy, the energy receiver receiving the transmitted wireless energy having a varied power. The electric circuit could be adapted to deliver the electrical pulses to remain unchanged except varying the first and/or second time intervals.

The system could according to one embodiment have an electric circuit having a time constant which is adapted to vary the first and second time intervals only in the range of the first time constant, so that when the lengths of the first and/or second time intervals are varied, the transmitted power over the coil is varied.

The implantable internal energy receiver for receiving wireless energy could comprise an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off.

The system could also comprise an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factors between the first and second coils.

In the embodiments in which the system comprises an external second coil, the external second coil could be adapted to be moved in relation to the internal first coil to establish the optimal placement of the second coil, in which the coupling factor is maximized. The external second coil could also be adapted to calibrate the amount of transferred energy to achieve the feedback information in the determination device, before the coupling factor is maximized.

According to a second aspect, a method of implanting the medical device according to any of the embodiments herein is further provided. The method comprises the steps of creating an opening reaching from outside of the human body into a joint, providing the artificial contacting surface to the joint, fixating the artificial contacting surface to the joint, implanting the reservoir in the human body, and lubricating the artificial contacting surface with use of a lubricating fluid contained in the reservoir.

The step of lubricating the joint contacting surface or the artificial contacting surface with use of a lubricating fluid contained in the reservoir could comprise implanting an operation device adapted to transport the fluid from the reservoir to the artificial contacting surface. According to another embodiment the step of lubricating the artificial contacting surface with use of a lubricating fluid contained in the reservoir comprises providing an energy source for powering the operation device.

According to yet another embodiment the step of lubricating the joint contacting surface or artificial contacting surface with use of a lubricating fluid contained in the reservoir could comprise powering the operation device using the energy source.

The step of implanting a reservoir in the human body could, according to one embodiment, comprise the step of implanting an operation device being integrated in the reservoir, allowing the step of lubricating the artificial contacting surface with use of a lubricating fluid contained in the reservoir, using the operation device transporting the fluid from the reservoir to the artificial contacting surface.

Implanting the reservoir, according to any of the embodiments could comprise the step of implanting the reservoir at least partially inside of a bone of the patient, which could be the femoral bone of the patient, the tibia bone of the patient and/or the pelvic bone of the patient.

The step of providing the artificial contacting surface could comprise the step of providing the artificial contacting surface from the abdominal side of the pelvic bone.

The step of implanting the reservoir in the human body could comprise the step of implanting the reservoir subcutaneously. Placing the reservoir subcutaneously allows simple access to the reservoir and eliminates the need for a long conduit between an injection port and the reservoir.

The step of implanting the reservoir subcutaneously could comprise the step of implanting the reservoir in at least one of the regions of the patient selected from a group of regions consisting of the abdominal region, the inguinal region, the pelvic region, the thigh region, and the calf region.

A further step of implanting an injection port for filling of the reservoir could be performed. The implantation of an injection port could comprise the step of implanting the injection port in connection with bone.

According to one embodiment, the medical device comprises an artificial contacting surface adapted to carry weight in a joint of a patient, the artificial contacting surface could comprise at least one channel for transporting a lubricating fluid, the method comprises the steps of implanting the medical device in a joint of the human patient, implanting a conduit adapted to be connected to the medical device, implanting an operation device for transporting a lubricating fluid inside the conduit, implanting a reservoir adapted to hold a lubricating fluid, and at least postoperatively transporting, by the operation device, the lubricating fluid from the reservoir to the artificial contacting surface in the conduit and further through the channel in the artificial contacting surface, thereby applying the lubricating fluid to the artificial contacting surface.

Generally, the lubrication device may be implanted during a conventional surgery or by endoscopic or laparoscopic methods. Further, one has to differentiate between methods for implanting a lubrication device having an infusion needle for intermittent introduction of lubricating fluid and methods for implanting a lubrication device having an infusion tube for continuous introduction of lubricating fluid.

In a method of treating a human or mammal joint, e.g. a human hip or knee joint osteoarthritis, by providing a lubricating fluid to the joint by means of the implantable lubrication device, a proper location including an area of the joint is dissected free in the patient's body by surgery, which may especially include cutting the patient's skin and dissecting a suitable place for a reservoir to store the lubricating fluid. Then, the lubrication device is placed at the dissected-free proper location in such a way that the fluid connection may post-operatively introduce lubricating fluid into the joint. For this purpose, a hole is created in the joint capsule at the dissected-free area of the joint and an infusion tube is introduced into the hole such that an open end of the infusion tube is placed in continuous communication with the joint in order to post-operatively inject lubricating fluid stored in the reservoir into the joint on a continuous basis. That is, the infusion tube is inserted in the hole such that, firstly, the opening end of the infusion tube is kept in permanent communication with the joint to be lubricated and, secondly, the infusion tube is in contact with the fluid connection device and thus with the reservoir. After placement of the lubrication device, the patient's body is closed such that the lubrication device is entirely implanted in the patient's body. This process may preferably be performed in layers and by means of sutures or staples or adhesives or the like. Finally, after the implantation process, the lubricating fluid is post-operatively introduced into the reservoir such that by operation of the implanted lubrication device the joint is adequately lubricated.

Alternatively, if the fluid connection comprises an intermittently operating infusion needle as the infusion member, the placement of the lubrication device at the dissected-free proper location and the area of the joint is realized by placing the infusion needle in such a close relation to the dissected area of the joint that a drive mechanism of the infusion needle may introduce and retract the infusion needle intermittently into/out of the joint such that lubricating fluid stored in the reservoir is intermittently injected into the joint. That is, the infusion needle is placed in close relation to the dissected-free area of the joint such that it may by intermittently introduced into the joint for lubricating the joint and retracted thereafter by an appropriate drive mechanism connected to a drive mechanism or the like.

Another method of treating a human or mammal patient by means of the implantable lubrication device utilizes endoscopic or laparoscopic techniques for creating an area of the joint via which lubricating fluid may be injected into the joint by the infusion member. This area of the joint is provided by, first, expanding a cavity in close relation to the joint by inserting a needle-like or a tube-like instrument in the patient's body and introducing a gas through the needle/tube-like instrument to fill gas into the tissue and thereby expand the cavity near the joint. Thereafter, at least two laparoscopic/endoscopic trocars are placed in the cavity and a camera and at least one dissecting tool are inserted through the laparoscopic trocars. The area of the joint is then dissected with the inserted dissecting tool. Also, a proper location for the remaining components of the lubrication device is dissected free, e.g. the reservoir, a pump or motor, or the like. The lubrication device is then placed at the proper location, whereas the fluid connection with the infusion member is arranged at the laparoscopically dissected area of the joint such that lubricating fluid is introduced into the joint. After placement of the lubrication device, the patient's body is closed with the effect that the lubrication device is entirely implanted in the patient's body. Thereafter, the lubricating fluid can be post-operatively introduced into the reservoir such that said joint is adequately lubricated through the fluid connection device and the infusion member.

Using the laparoscopic approach, again, a lubrication device having either an infusion tube or an infusion needle may be implanted. In the former case, the reservoir is placed at the proper location and a hole is created in the joint capsule at the laparoscopically dissected area of the joint and the infusion tube is inserted into the hole such that that an open end of the tube is placed in continuous communication with the joint and the stored lubricating fluid may continuously be injected into the joint. In the latter case, after placing the reservoir at the proper location, an infusion needle and a drive mechanism are placed in close relation to the laparoscopically dissected area of the joint such that the drive mechanism may intermittently introduce (and retract) the infusion needle into (and out of) the joint in order to allow the stored lubricating fluid to be intermittently injected into the joint.

Closing the patient's body, or particularly the skin, may for instance include suturing, taping and other suitable techniques. The lubrication device may be placed subcutaneously in the patient's body or within fat tissue or intramuscularly. If appropriate, the lubrication device may also be placed within or adjacent the patient's gastro-intestinal or urinary tract. When it is placed adjacent the tract, it may be secured to the gastro-intestinal or urinary tract by means of a holder connected to the lubrication device. As a further alternative, the lubrication device may be placed in the patient's thorax or in the patient's abdomen. For instance, a reservoir may be placed in the abdomen or thorax cavity. Alternatively, the lubrication device or part thereof, such as a reservoir, may be implanted by open surgery, in which case the thorax or abdominal wall is opened for placing the lubrication device at the proper location within the patient's thorax or abdomen and, afterwards, the skin and other layers of tissue are closed, such as by suturing, being preferably sutured in layers. Replenishing of the reservoir preferably comprises the step of injecting a volume of lubrication liquid through the injection port connected to and/or integrated in the periphery of the reservoir, e.g. a reservoir.

Functional hip movements are to be understood as movements of the hip that at least partly correspond to the natural movements of the hip. On some occasions the natural movements of the hip joint might be somewhat limited or altered after hip joint surgery, which makes the functional hip movements of a hip joint with artificial surfaces somewhat different than the functional hip movements of a natural hip joint.

The functional position of an implantable medical hip device or prosthesis is the position in which the hip joint can perform functional hip movements. The final position is to be understood as a functional position in which the medical device needs no further position change.

Functional knee movements are to be understood as movements of the knee that at least partly correspond to the natural movements of the knee. On some occasions the natural movements of the knee joint might be somewhat limited or altered after knee joint surgery, which makes the functional knee movements of a knee joint with artificial surfaces somewhat different than the functional knee movements of a natural knee joint.

The functional position of an implantable medical knee device or prosthesis is the position in which the knee joint can perform functional knee movements.

Functional knee joint is a knee joint that can perform functional knee movements either with or without an implanted medical device or prosthesis.

Full functional size is to be understood as the size of the medical knee device when said medical device is implanted in the knee joint.

Arthroscopy is to be understood as key hole surgery performed in a joint, since the arthroscopic procedure could be performed in the abdomen of the patient some of the steps ofthis arthroscopic procedure is more laparoscopic, however for the purpose of this invention the two terms arthroscopy and laparoscopy is used synonymously and for the purpose of this invention the main purpose of these methods are is that they are minimally invasive.

The medical device according to any of the embodiments could comprise at least one material selected from a group consisting of: polytetrafluoroethylene (PTFE), perfluoroalkoxy (PFA) and fluorinated ethylene propylene (FEP). It is furthermore conceivable that the material comprises a metal alloy, such as cobalt-chromium-molybdenum or titanium or stainless steel, or polyethylene, such as cross-linked polyethylene or gas sterilized polyethylene. The use of ceramic material is also conceivable, in the artificial contacting surfaces or the entire medical device such as zirconium or zirconium dioxide ceramics or alumina ceramics. The part of the medical device in contact with human bone for fixation of the medical device to human bone could comprise a poorhouse structure which could be a porous micro or nano-structure adapted to promote the growth-in of human bone in the medical device for fixating the medical device. The porous structure could be achieved by applying a hydroxy-apatite (HA) coating, or a rough open-pored titanium coating, which could be produced by air plasma spraying, a combination comprising a rough open-pored titanium coating and a HA top layer is also conceivable. The contacting parts could be made of a self-lubricated material such as a waxy polymer, such as PTFE, PFA, FEP, PE or UHMWPE, or a powder metallurgy material which could be infused with a lubricant, which preferably is a biocompatible lubricant such as a Hyaluronic acid derivate. It is also conceivable that the material of contacting parts or surfaces of the medical device herein is adapted to be constantly or intermittently lubricated. According to some embodiments the parts or portions of the medical device could comprise a combination of metal materials and/or carbon fibers and/or boron, a combination of metal and plastic materials, a combination of metal and carbon based material, a combination of carbon and plastic based material, a combination of flexible and stiff materials, a combination of elastic and less elastic materials, Corian or acrylic polymers.

### F: Control and Adjustments of a Bone Adjustment in a Mammal

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control and adjustments of a bone adjustment in a mammal. Examples of such devices for bone adjustments will now be described.

The inventions concern a device for bone adjustment in a mammal, which according to an embodiment comprises two or more anchoring devices for attaching to a bone in said mammal, and an adjustment device for exerting force on said anchoring devices to adjust the distance between or orientation of at least two of said anchoring devices, wherein said anchoring devices and said adjustment device are implanted intramedullary in said mammal and wherein said adjustment device is constructed to postoperatively adjust said distance.

In a device according to said embodiment, the adjustment is the lengthening of a bone, the healing of a fracture, the changing of a bone angle, the reshaping of a bone, the compression of a bone, the torsion of a bone or a combination thereof.

In a device according to any of the embodiments presented herein, said two or more anchoring devices are adapted to engage the bone from the inside of the intramedullar cavity. Preferably said at least two anchoring devices are chosen from a screw, an adhesive, a barb construction, a saw-tooth construction, an expandable element, combinations thereof or other mechanical connecting members.

In a device according to any of the embodiments presented herein, the force exerted by the adjustment device is a longitudinal force, extending the length of the bone.

According to an embodiment, said longitudinal force is directed to the end portions of the medullar cavity.

According to another embodiment, the force exerted by the adjustment device is a longitudinal force, adjusting the angle or curvature of the bone.

According to yet another embodiment, the force exerted by the device applies torque to the bone, adjusting the torsion of the bone along its longitudinal axis.

According to another embodiment, freely combinable with any of the embodiments herein, said device is flexible.

According to another embodiment, the adjustment device comprises a hydraulic device for said bone adjustment, to control the amount of force exerted by the device onto said anchoring devices. Preferably said hydraulic device comprises a cylinder and piston.

According to another embodiment, the hydraulic device comprises a mechanical multi step locking mechanism, locking the hydraulic device in its new position after adjustment. Said mechanical multi step locking mechanism may comprise at least one of a sprint, an elongated structure using the principle of saw teeth, flanges, barbs or a bonnet band, a nut, a gearbox, or a spring loaded locking principle.

According to another embodiment, the hydraulic device comprises hydraulic fluid and a reservoir containing said fluid, adapted to move said fluid to said adjustment device. Preferably said hydraulic fluid is moved from said reservoir to said adjustment device by using a pre-pressurized reservoir or a pump.

According to another embodiment, the hydraulic device comprises a device positioning system such as a fluid volume or flow measurement or any other sensor input to see the position of the adjustment device.

According to another embodiment, the device comprises a control device. Preferably said control device follows a program of incremental changes, set before the device is implanted. Alternatively, said control device follows a program of incremental changes, communicated to the control device after implantation and/or during the treatment. X-ray or ultrasonic follow-up may indicate further treatment, decided by the responsible orthopaedic surgeon.

According to another embodiment, said control device comprises an external control unit and an implantable receiver suitable for wireless communication with said external control unit, having a transmitter located outside the body.

According to another embodiment, said control device controls incremental changes of the adjustment device, communicated to the receiver after implantation and/or during the treatment by using said external control unit.

According to another embodiment, freely combinable with any one of the embodiments presented herein, said hydraulic adjustment device is adapted to being stabilized when the bone adjustment is completed. In this embodiment, the hydraulic adjustment device can be filled with a material which stabilizes the position of the adjustment device and permanents the distance between the anchoring devices.

Preferably said material is chosen from curable foam, a curable gel, a polymer or polymer mixture which solidifies, crosslinks or otherwise attains and retains a stable volume. Alternatively, the hydraulic fluid used in said device is a material chosen from a curable foam, a curable gel, a polymer or polymer mixture which solidifies, crosslinks or otherwise attains and retains a stable volume when the curing, solidification, crosslinking or other reaction is initiated by the user. According to another embodiment, said material chosen from a curable foam, a curable gel, a polymer or polymer mixture which solidifies, crosslinks or otherwise attains and retains a stable volume, is added to the device, partially or completely replacing the hydraulic fluid.

When said material solidifies, further movement of the device is prevented, and the device can be left in place if desired. By the verb “fill” as in “the device can be filled” is meant that the inside of the device, e.g. the inside of a hydraulic device, such as the hydraulic chamber, piston, bellows or the like, is filled. In a mechanical device, the housing surrounding any movable parts is filled, preventing further movement of said parts.

According to another embodiment, the adjustment device comprises a mechanical device for said bone adjustment. Preferably said adjustment device is operated by an operation device, such as motor.

According to another embodiment, the adjustment device comprises a control device, wherein the operation device is controlled by said control device.

According to another embodiment, the motor comprises a motor or device positioning system such as a tachometer or any other sensor input to see the position of the adjustment device.

According to another embodiment, the mechanical device for said bone adjustment comprises at least one nut and screw.

According to another embodiment, the mechanical device for said bone adjustment comprises at least one gearbox.

According to yet another embodiment, the mechanical device for said bone adjustment comprises a servo mechanism or mechanical amplifier.

According to any one of the embodiments herein, the device is adapted for exerting an intermittent and/or oscillating force. It is here noted that a method for distractive osteogenesis, where the fractured bone is subjected to an intermittent and/or oscillating force using an implanted hydraulic device, is a particularly preferred embodiment of the invention. The device according to the invention is especially suitable for such treatment, as it requires minimal or no manual adjustment, and can exert its force or torque according to a preset program or according to instructions transmitted wirelessly to the device.

According to any one of the embodiments herein, the device comprises a locking device which allows extension of the device but substantially prevents contraction.

Another embodiment of the inventions is a method for bone adjustment in a mammal, wherein a hydraulic or mechanical device according to any one of the above embodiments is used and implanted in the body of said mammal.

According to another embodiment, the device is implanted intramedullary in the body of said mammal, exerting a force to anchoring devices anchored to the inside of said bone.

According to another embodiment of the method, said bone adjustment is the lengthening of a bone, the healing of a fracture, the changing of a bone angle, the reshaping of a bone, the torsion of a bone or a combination thereof.

According to one embodiment, said adjustment is a step in a treatment to correct a limb discrepancy caused by a congenital condition, deformation or previous trauma. For a better understanding of the field, a non-exclusive list of examples is given in Table 1 below. It is conceived that the device and method according to the invention can be applied by a skilled person to any of these, given that the necessary modifications with regard to size, force and location are made. Such modifications however appear to be within the realm of a skilled person without an inventive effort.

### Table 1. Examples of Conditions Contemplated as Possible to Treat with a Device and Method According to the Invention

Congenital deformities (birth defects), such as congenital short femur; fibular hemimelia (absence of the fibula, which is one of the two bones between the knee and the ankle); hemiatrophy (atrophy of half of the body); and Ollier's disease (also known as multiple endochondromatosis, dyschondroplasia, and endochondromatosis).
Developmental deformities, such as neurofibromatosis (a rare condition which causes overgrowth in one leg); and bow legs, resulting from rickets (rachitis) or secondary arthritis.
Post-traumatic injuries, such as growth plates fractures; malunion or non-union (when bones do not completely join, or join in a faulty position after a fracture); shortening and deformity; and bone defects.
Infections and diseases, such as osteomyelitis (a bone infection, usually caused by bacteria); septic arthritis (infections or bacterial arthritis); and poliomyelitis (a viral disease which may result in the atrophy of muscles, causing permanent deformity).
Reconstruction after removal of tumours.
Short stature, such as achondroplasia (a form of dwarfism where arms and legs are very short, but torso is more normal in size); and constitutional short stature.

According to another embodiment, said adjustment is reshaping or lengthening of a bone involving distraction osteogenesis treatment. Distraction osteogenesis can also be applied to cosmetic lengthening of limbs, although healthy patients are currently often discouraged to undertake such treatments due to the risk of complications, and the cost and suffering involved. It is underlined that the embodiments of the present invention are applicable to both therapeutic and cosmetic uses.

According to yet another embodiment, said adjustment is the reshaping or lengthening of a bone as a step of correcting a congenital deformation.

According to a further embodiment, said adjustment is the reshaping or lengthening of a bone as a step of a cosmetic treatment.

According to any of the above embodiments of the method, reshaping is one of changing the angle or curvature of a bone, changing the torsion of a bone, changing the angle between the diaphysis and the epiphysis, changing the thickness of a bone or a combination thereof.

Another embodiment of the inventions is a method for bone adjustment in a mammal wherein a device is implanted intramedullary in the body of said mammal, wherein said device is a hydraulic device exerting a force to anchoring devices anchored in said bone and a control device which controls the amount of force exerted by the device.

Another embodiment of the inventions is a method for bone adjustment in a mammal wherein a device is implanted intramedullary in the body of said mammal, wherein said device is a mechanical device exerting a force to anchoring devices anchored in said bone and a control device which controls the amount of force exerted by the device.

In any of the above methods, said control device follows a program of incremental changes, set before the device is implanted. Alternatively, said control device follows a program of incremental changes, communicated to the control device after implantation and/or during the treatment.

According to any of the embodiments of the method, said device is stabilized when the treatment is completed. Preferably said device is stabilized by filling the device with a material which stabilizes the position of the adjustment device and permanents the distance between the anchoring devices. In this method said material is chosen from a curable foam, a curable gel, a polymer or polymer mixture which solidifies, crosslinks or otherwise attains and retains a stable volume.

Alternatively, the device is a hydraulic device and the hydraulic fluid is a material chosen from a curable foam, a curable gel, a polymer or polymer mixture which solidifies, crosslinks or otherwise attains and retains a stable volume when the curing, solidification, crosslinking or other reaction is initiated by the user.

According to another embodiment, the device is a hydraulic device and a material chosen from a curable foam, a curable gel, a polymer or polymer mixture which solidifies, crosslinks or otherwise attains and retains a stable volume, is added to said device, partially or completely replacing the hydraulic fluid.

Another embodiment of the inventions is a method for distractive osteogenesis where the fractured bone is subjected to an intermittent and/or oscillating force using an implanted hydraulic or mechanical device.

Another embodiment of the inventions is a method for treating a bone dysfunction of a mammal patient by providing a device for bone adjustment comprising at least two anchoring devices according to any one of embodiments of a device according to the inventions, the method comprising the steps of
i. inserting a needle or tube-like instrument into a cavity of said mammal patient;
ii. inflating said cavity by introducing a fluid through said needle or tube-like instrument and thereby expanding said cavity;
iii. placing at least two laparoscopic trocars in said cavity;
iv. inserting a camera through one of said laparoscopic trocars into said cavity;
v. inserting at least one dissecting tool through one of said at least two laparoscopic trocars;
vi. dissecting an area of the dysfunctional bone;
vii. placing the device for bone adjustment and anchoring devices in the medullar cavity of said bone;
viii. anchoring said anchoring devices in contact with said bone;
ix. closing the mammal body preferably in layers; and
x. non-invasively adjusting said bone postoperatively.

Another embodiment of the inventions is a method of treating a bone dysfunction of a mammal patient by providing a device for bone adjustment comprising at least two anchoring devices according to any one the embodiments of the device according to the inventions, comprising the steps of:
i. cutting the skin of said human patient;
ii. dissecting an area of the dysfunctional bone;
iii. placing the device in the medullar cavity of said bone;
iv. anchoring said anchoring devices in contact with said bone;
v. closing the mammal body preferably in layers; and
vi. non-invasively adjusting said bone postoperatively.

According to a further embodiment, the method of treating a mammal patient further comprises the step of withdrawing the instruments.

According to a further embodiment, the method of treating a mammal patient further comprises the step of closing the skin using sutures or staples.

According to a further embodiment, the step of dissecting includes dissecting an area of the arm or leg comprising, dissecting an area of at least one of the following bones; clavicula, scapula, humerus, radius, ulna, pelvic bone, femur, tibia, fibula or calcaneus.

According to a further embodiment, the step of dissecting includes dissecting an area of the arm or leg comprising, dissecting an area at least one of the following joints; shoulder, elbow, hip, knee, hand and foot.

According to a further embodiment of the method, an opening into the medullar cavity is made by drilling.

Another embodiment of the inventions relates to a system comprising an apparatus or device according to any one of the embodiments presented herein.

According to a further embodiment, said system further comprises at least one switch implantable in the patient for manually and non-invasively controlling the apparatus.

According to a further embodiment, said system further comprises a hydraulic device having an implantable hydraulic reservoir, which is hydraulically connected to the apparatus, wherein the apparatus is adapted to be non-invasively regulated by manually pressing the hydraulic reservoir.

According to a further embodiment, said system further comprises a wireless remote control for non-invasively controlling the apparatus.

According to an embodiment, said wireless remote control comprises at least one external signal transmitter and/or receiver, further comprising an internal signal receiver and/or transmitter implantable in the patient for receiving signals transmitted by the external signal transmitter or transmitting signals to the external signal receiver.

According to an embodiment, said wireless remote control transmits at least one wireless control signal for controlling the apparatus.

According to an embodiment, said wireless control signal comprises a frequency, amplitude, or phase modulated signal or a combination thereof.

According to an embodiment, said wireless remote control transmits an electromagnetic carrier wave signal for carrying the control signal.

According to yet another embodiment, said system further comprises a wireless energy-transmission device for non-invasively energizing implantable energy consuming components of the apparatus with wireless energy.

According to an embodiment, said wireless energy comprises a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal.

According to an embodiment, said wireless energy comprises one of the following: an electric field, a magnetic field, a combined electric and magnetic field.

According to an embodiment, said control signal comprises one of the following: an electric field, a magnetic field, a combined electric and magnetic field.

According to a further embodiment, said the signal comprises an analogue signal, a digital signal, or a combination of an analogue and digital signal.

According to yet another embodiment, said system further comprises an implantable internal energy source for powering implantable energy consuming components of the apparatus.

According to yet another embodiment, said system further comprises an external energy source for transferring energy in a wireless mode, wherein the internal energy source is chargeable by the energy transferred in the wireless mode.

According to yet another embodiment, said system further comprises a sensor or measuring device sensing or measuring a functional parameter correlated to the transfer of energy for charging the internal energy source, and a feedback device for sending feedback information from inside the patient's body to the outside thereof, the feedback information being related to the functional parameter sensed by the sensor or measured by the measuring device.

According to yet another embodiment, said system further comprises a feedback device for sending feedback information from inside the patient's body to the outside thereof, the feedback information being related to at least one of a physical parameter of the patient and a functional parameter related to the apparatus.

According to yet another embodiment, said system further comprises a sensor and/or a measuring device and an implantable internal control unit for controlling the apparatus in response to information being related to at least one of a physical parameter of the patient sensed by the sensor or measured by the measuring device and a functional parameter related to the apparatus sensed by the sensor or measured by the measuring device.

According to another embodiment, said physical parameter is a pressure or a motility movement.

According to yet another embodiment, said system further comprises an external data communicator and an implantable internal data communicator communicating with the external data communicator, wherein the internal communicator feeds data related to the apparatus or the patient to the external data communicator and/or the external data communicator feeds data to the internal data communicator.

According to yet another embodiment, said system further comprises a motor or a pump for operating the apparatus.

According to yet another embodiment, said system further comprises a hydraulic operation device for operating the apparatus.

According to yet another embodiment, said system further comprises an operation device for operating the apparatus, wherein the operation device comprises a servo designed to decrease the force needed for the operation device to operate the apparatus instead the operation device acting a longer way, increasing the time for a determined action.

According to yet another embodiment, said system further comprises an operation device for operating the apparatus, wherein the wireless energy is used in its wireless state to directly power the operation device to create kinetic energy for the operation of the apparatus, as the wireless energy is being transmitted by the energy-transmission device.

According to yet another embodiment, said system further comprises an energy-transforming device for transforming the wireless energy transmitted by the energy-transmission device from a first form into a second form of energy.

According to an embodiment, said energy-transforming device directly powers implantable energy consuming components of the apparatus with the second form energy, as the energy-transforming device transforms the first form energy transmitted by the energy-transmission device into the second form energy.

According to an embodiment, said second form energy comprises at least one of a direct current, pulsating direct current and an alternating current.

According to yet another embodiment, said system further comprises an implantable accumulator, wherein the second form energy is used at least partly to charge the accumulator.

According to an embodiment, said energy of the first or second form comprises at least one of magnetic energy, kinetic energy, sound energy, chemical energy, radiant energy, electromagnetic energy, photo energy, nuclear energy thermal energy, non-magnetic energy, non-kinetic energy, non-chemical energy, non-sonic energy, non-nuclear energy and non-thermal energy.

According to yet another embodiment, said system further comprises implantable electrical components including at least one voltage level guard and/or at least one constant current guard.

According to yet another embodiment, said system further comprises a control device for controlling the transmission of wireless energy from the energy-transmission device, and an implantable internal energy receiver for receiving the transmitted wireless energy, the internal energy receiver being connected to implantable energy consuming components of the apparatus for directly or indirectly supplying received energy thereto, the system further comprising a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

According to an embodiment, said determination device is adapted to detect a change in the energy balance, and the control device controls the transmission of wireless energy based on the detected energy balance change.

According to a further embodiment, the determination device is adapted to detect a difference between energy received by the internal energy receiver and energy used for the implantable energy consuming components of the apparatus, and the control device controls the transmission of wireless energy based on the detected energy difference.

According to a further embodiment, the energy-transmission device comprises a coil placed externally to the human body, further comprising an implantable energy receiver to be placed internally in the human body and an electric circuit connected to power the external coil with electrical pulses to transmit the wireless energy, the electrical pulses having leading and trailing edges, the electric circuit adapted to vary first time intervals between successive leading and trailing edges and/or second time intervals between successive trailing and leading edges of the electrical pulses to vary the power of the transmitted wireless energy, the energy receiver receiving the transmitted wireless energy having a varied power.

According to a further embodiment, the electric circuit is adapted to deliver the electrical pulses to remain unchanged except varying the first and/or second time intervals.

According to a further embodiment, the electric circuit has a time constant and is adapted to vary the first and second time intervals only in the range of the first time constant, so that when the lengths of the first and/or second time intervals are varied, the transmitted power over the coil is varied.

According to a further embodiment, the system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off.

According to a further embodiment, the system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factors between the first and second coils.

According to an embodiment, the transmitted energy may be regulated depending on the obtained coupling factor.

According to a further embodiment, said external second coil is adapted to be moved in relation to the internal first coil to establish the optimal placement of the second coil, in which the coupling factor is maximized.

According to a further embodiment, said external second coil is adapted to calibrate the amount of transferred energy to achieve the feedback information in the determination device, before the coupling factor is maximized.

According to yet another embodiment, the mechanical device comprises a mechanical multi step locking mechanism, locking the mechanical device in its new position after adjustment.

According to yet another embodiment, the mechanical multi step locking mechanism comprises at least one of a sprint, an elongated structure using the principle of saw teeth, flanges, barbs or a bonnet band, a nut, a gearbox, or a spring loaded locking principle.

According to a further embodiment of the system, the device comprises a control device.

According to another embodiment, said control device follows a program of incremental changes, set before the device is implanted.

According to another embodiment, said control device follows a program of incremental changes, communicated to the control device after implantation and/or during the treatment.

According to another embodiment, said control device comprises an external control unit and an implantable receiver suitable for wireless communication with said external control unit, having a transmitter located outside the body.

According to another embodiment, said control device controls incremental changes of the adjustment device, communicated to the receiver after implantation and/or during the treatment by using said external control unit.

According to a preferred embodiment, said device is flexible to allow introduction into the medullar cavity. Alternatively or in combination therewith, said device is at least partly elastic.

Alternatively or in combination therewith, said device comprises a spring. Alternatively or in combination therewith, said device is adapted to regain its shape after having been bent.

According to another embodiment, freely combinable with the other embodiments presented herein, the anchoring device comprises a thread for engaging and stabilizing the anchoring device in relation to the bone.

According to a further embodiment, the anchoring device comprises an expandable part expanding at least partially perpendicular to the longitudinal extension of the elongated device for engaging and stabilizing the anchoring device in relation to the bone.

According to a further embodiment, the adjustment device is adapted to comprise torsion of a bone. Alternatively, or in combination, said adjustment device is adapted to change the angle of a bone.

According to a further embodiment, said adjustment device comprises at least two parts, wherein the parts are adapted to rotate in relation to each other. Preferably said relative rotation is anchored by said at least two anchoring devices.

According to another embodiment, freely combinable with the other embodiments presented herein, said adjustment device is adapted to change the angle of a bone.

According to further embodiment, freely combinable with the other embodiments presented herein, said adjustment device comprises at least two parts, wherein the parts are adapted to be positioned at an angle in relation to each other.

According to a further embodiment, said two or more anchoring devices are adapted to engage and carry weight purely on the inside of the bone.

According to yet another embodiment, said two or more anchoring devices are adapted to engage with and carry weight to the bone without penetrating to the outside of the bone.

According to yet another embodiment, said two or more anchoring devices are adapted to engage and carry weight purely on the outside of the bone.

According to another embodiment, freely combinable with any embodiment presented herein, said device comprises a sensor directly or indirectly sensing the position of the adjustment device.

According to a further embodiment, the device comprises a feedback transmitter adapted to transmit information received directly or indirectly from said sensor out from the human body, said transmitted information adapted to be received by an external control unit and relating to the position of the adjustment device.

According to another embodiment of the device, said operation device is a motor operated as a three-phase motor. Alternatively, said operation device is a motor operated as a two- or more phase motor.

According to another embodiment, freely combinable with any embodiment presented herein, said device comprises a gearbox connected to the motor, a motor package, wherein the outgoing speed from the motor package is lower than the speed by said motor alone, accomplished by said gearbox.

According to another embodiment, freely combinable with any embodiment presented herein, said device comprises an electrical speed controller connected to the motor, a motor package, wherein the outgoing speed of the motor in said motor package is decreased by said electrical speed controller.

According to any of the above embodiments, the motor is a rotational motor and the outgoing speed of the motor package is decreased to less than 100 turns per second, alternatively decreased to less than 10 turns per second, alternatively to less than 1 turn per second, or alternatively to less than 0.1 turn per second, or alternatively to less than 0.01 turn per second, or alternatively to less than 0.001 turn per second.

According to another embodiment, freely combinable with any embodiment presented herein, said device comprises an electrical speed controller connected to the motor, a motor package, wherein the outgoing speed of the motor of said motor package is controlled by said electrical speed controller.

According to any of the above embodiments, the motor is a linear motor and the outgoing speed of the motor package is less than 1 mm per second, alternatively less than 0.1 mm per second, or alternatively less than 0.01 mm per second, or alternatively less than 0.001 mm per second, or alternatively less than 0.0001 mm per second, or less than 0.00001 mm per second.

One advantage of the device and method according to the inventions is that the need for manual adjustment of the device is removed. In a traditional, external apparatus, the distance of the elements is adjusted manually, in increments. It is conceived that each incremental adjustment is associated with pain, whereas an automatic, constant adjustment may be better tolerated.

Another advantage is that the implanted device can exert an oscillating or intermittent force without the need for manual adjustments, or the need of connecting the patient to an external apparatus.

Yet another advantage is that the number and size of visible elements outside the body is minimized. This is an important aesthetic and practical improvement, which also has social and psychological benefits, as the patient becomes less exposed and attracts less attention. A provision of an implanted device may also help the patient to feel intact and healthier, thus promoting a positive mental attitude and promoting healing.

It should be noted that the above embodiments, and features appearing in the individual embodiments, are freely combinable.

### G: Drain—Assisting Control Fluid Movement Devices

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control fluid movement devices, such as drainage devices or hydraulic treatment devices.

It is another object of the present invention to provide a fluid movement device that enables a patient to more easily move around while still being attached to the drain.

It is yet another object to provide a fluid movement device that can be efficiently secured.

At least one of the above objects is obtained by the method, apparatus, device and system as set out in the appended claims. Thus, by providing an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.

The apparatus for drainage of a body fluid or movement of hydraulic treatment fluid in a human or mammal patient in accordance with the present invention comprises a fluid movement device for pumping hydraulic treatment fluid or body fluid. The fluid movement device is powered by an energy source and may be powered by any suitable means such as an electrical or a hydraulic motor. At least one connecting tube is connected to the fluid movement device so that the fluid movement device and the tube form a drainage or hydraulic arrangement. The arrangement is adapted to be implanted inside the body of the patient, and placed so that the tube interconnects one part of the body with another part of the body and where fluid movement device is adapted to suck body fluid from the one part of the body via the tube to the other part of the body. Hereby an implantable fluid movement device is obtained which can pump body or hydraulic fluid from a treatment area to another part of the body where the fluid can be absorbed or transported out from the body in a normal way.

The implantable fluid movement device in accordance with the present invention can be used to move body fluid between different parts of the body depending on the type of body fluid being drained. For example and without limitation the fluid movement device can be adapted to drain urine from the urine accumulating renal part of the kidney, and moving the urine via at least one tube to the urine bladder. The fluid movement device can also be adapted to drain liquid from the hydrocephalus in the brain area, and moving it to the abdomen. The fluid movement device can also be adapted to drain liquid from ascites in the abdomen, and moving it to the lymphatic system of the body or to the urine bladder. Also, the fluid movement device can also be adapted to drain liquid from the thoraxial cavity, and moving the liquid to the abdomen.

Depending on the type of treatment and where the body fluid is sucked from and to where in the body the fluid is delivered the tubes used may be shaped to suit the particular treatment.

In accordance with one embodiment a method of securing a connecting tube for use in an implantable device is provided. The tube is adapted to move body fluid from one part of the body, via the at least one connecting tube to another part of the body, the connecting tube having a distal end adapted to be located in the bladder of the human or mammal patient for drainage of a body fluid or hydraulic treatment fluid from a treatment area of the human or mammal patient into the bladder, the method comprising the steps of:
opening a hole in the bladder,
placing the end of the tube in the bladder.
securing the tube on the outside of the bladder by invaginating the tube using sutures or staples, thus creating a tunnel around the tube, wherein said tube comprising a net material secured to said tube, in the part of the tube end at a distance from said tube end, the further method comprising,
placing the net material in connection to the opening of the invaginated tunnel, and
securing the net material to the outside of the bladder.

The bladder can be the urine bladder or the peritoneum. The same method can also be used for securely fastening a tube into other organs.

In accordance with one embodiment a tube adapted to be inserted in a luminal or bladder organ of a patient, said tube adapted to enter said organ in a tube passageway. The tube comprises a combined securing and sealing device adapted for long term closing of the tube passageway and for long term securing the tube onto an organ. The combined securing and sealing device can comprise a patch comprising a net mounted onto the tube. The net can be adapted to a seal of overgrowth of human fibrotic tissue over the whole net and the patched part of said organ, thereby completely sealing said net and attaching said net to said organ, thus sealing around said tubular passageway. In accordance with one embodiment a net structure is provide with openings less than 2.5 mm, preferable 0.5 mm, to allow said tissue overgrowth.

A specific embodiment of a hydraulic treatment system is presented below.

In one embodiment an apparatus for treating urinary retention of a patient by discharging urine from the urinary bladder, comprising an expandable member adapted to be implanted inside the urinary bladder of the patient, and an implantable control device for controlling the volume of the expandable member, the control device being adapted to be connected to the expandable member through the wall of the urinary bladder is provided. As a result of the expansion of the expandable member urine is discharged from urinary bladder through the urethra.

The expandable member is preferably releasably attached to the control device with quick coupling, such as snap-lock fitting or the similar and further member is designed with a capacity to assume a shape which admits its transportation through urethra. The expandable member can comprise a bellow or a similar structure undergoing controlled expansion and collapse.

Further, the expandable member is hydraulically controlled and comprises a cavity for hydraulic fluid and the control device comprises a reservoir for hydraulic fluid. The expandable member and the control device are accordingly adapted to be hydraulically connected through the wall of urinary bladder. For this purpose, the control device preferably comprises a tube to establish hydraulic connection and for transporting the hydraulic fluid between the reservoir and the cavity.

The control device of the apparatus can comprises an operation device for transporting hydraulic fluid to and from the cavity and the reservoir. In one mode of operation, the expandable member is adapted to be emptied by the pressure exerted by urine of the urinary bladder to transport the hydraulic fluid from the cavity to the reservoir. The operation device is capable of transporting hydraulic fluid to cavity of the expandable member to obtain a suitable urinary pressure for discharging urine. Also a urinary pressure of at least 50 cm water pressure for discharging urine can be provided.

The operation device can be powered, In one embodiment the operation device is a powered pump. Further, the operation device can comprise or being connected to an injection port, to calibrate the amount of hydraulic fluid. The operation device can also be manually operated by an injection port which is operated from outside the body by filling or emptying said injection port.

In addition the apparatus can comprise implantable restriction devices adapted to close the ureters when discharging urine from the urinary bladder in order to prevent any urinary backflow towards the kidneys. Preferably, these restriction devices open and close by the activity of the operation device.

The apparatus can also comprise a restriction device adapted to open and close the urethra to assist patients having an impaired urinary sphincter function.

The control device can further comprise a control assembly adapted to be implanted subcutaneously or in the abdominal cavity in the patient for connection to other parts of the control device. The control assembly comprises a source of energy for powering the operation device and other energy consuming parts of the control device. These parts are further described in the context of the system according to invention comprising the recited apparatus. The control assembly can further comprise an injection port for receiving hydraulic fluid, connected to the reservoir.

The apparatus can also comprise an implantable pressure sensor for measuring the urinary pressure in the urinary bladder direct or indirect, such as measuring the pressure inside the implantable member.

The hydraulic fluid can comprise an agent for counteracting microbial growth, such as an antibiotic.

In order to further assist urinary discharge, the control device can further comprise an implantable device for electrically stimulating muscles of the urinary bladder to contract the same, to co-operate with the expandable member to discharge urine from the urine bladder.

Also, the electrically stimulating device can comprise a plurality of electrode strips attached to muscles of the urinary bladder. In an alternative, the apparatus can comprise a second hydraulic connection between the expandable member and the reservoir. The second connection is dimensioned so that the pumps pumping volume capacity is clearly much larger than the emptying capacity of said second connection, when open. According to this alternative arrangement, the expandable member is adapted to be emptied by the pressure exerted by urine of the urinary bladder to transport the hydraulic fluid from the cavity to the reservoir by said second connection.

The present invention also relates to a method implanting the described apparatus, which comprises inserting a needle-like tube into the abdomen of the patient; filling the abdomen with gas through said tube, thereby expanding the abdominal cavity; placing at least two laparoscopic trocars in the patient's body and inserting a camera through one of said trocars into the abdomen; inserting at least one dissecting tool through a trocar and dissecting an area of at least one portion of the urinary bladder of patient; incising an opening in the urinary bladder wall; placing an expandable member inside the urinary bladder; placing a control device outside the urinary bladder; and interconnecting the expandable member and the control device with an interconnection device. The method also comprises tunnelling by suturing the urinary bladder wall to itself in order to immobilize the interconnecting device in position penetrating the urinary bladder wall while establishing a hydraulic connection between a cavity of the expandable member and a reservoir of the control device. Further, the method comprises placing net adapted to support in-growth of tissue with so it at least partially covers the tunnelling.

The present invention further extends to a method of operating the apparatus according to any that comprises activating a control assembly of the control device; increasing the volume of the expandable member; and discharging urine through the urethra. The method can further comprises the step activating the restriction devices to temporarily close the ureters and/or a step comprising activating the restriction device to temporarily release its restriction of the urethra or the neck of the urine bladder. In the method a control assembly can receive a signal from a pressure sensor measuring the urinary pressure in the urinary bladder or expandable member, said control assembly comprising an alarm system adapted to present an alarm signal for the patient, being able to activate said control assembly with a signal from a control unit controlled from external to the patient, such as a wireless remote control or an subcutaneously implantable switch. The method can further comprise the step of activating a pump for transporting hydraulic fluid from said reservoir to the expandable member.

The present invention sin yet another embodiment extends to a method of replacing an expandable member in the previous described apparatus for treating urinary retention comprising the steps of inserting an instrument adapted to operate on the expandable member through the urethra;
releasing the expandable member from the control device; displacing the collapsed expandable member with the instrument; and transporting the collapsed expandable member through the urethra and out of the body. Further, the method comprises inserting a new, collapsed expandable member through the urethra; displacing the expandable member to a coupling position with control device; and attaching the expandable member to the control device with a quick coupling.

The present invention also relates to a system treating urinary incontinence comprising the previously described apparatus. Parts or components of system are described in the following sections of the description and should be regarded as applicable with any apparatus described above. In a one embodiment, the system comprises at least one switch implantable in the patient for manually and non-invasively controlling the apparatus In another embodiment, the system comprises a wireless remote control for non-invasively controlling the apparatus.

In one embodiment, the system comprises a hydraulic operation device for operating the apparatus.

In one embodiment, the system comprises comprising a motor or a pump for operating the apparatus.

### H: Assisting the Pump Function of the Heart

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for assisting the pump function of the heart, examples of such devices for assisting the pump function of the heart will now be described.

According to one embodiment an implantable device for improving the pump function of the heart of a human patient by applying an external force on the heart muscle, said device comprising at least one heart contacting organ, periodically exerting force onto the heart following the heart contractions and adding force thereto, said implantable device adapted to have a drive unit to create kinetic movement to be used by the heart contacting organ, wherein said implantable device comprising a fixation device adapted to be mounted in a stable position to human bone allowing said drive unit and kinetic movement to get necessary contra force, wherein said drive unit further comprising a respiration movement compensator for compensating for the respiratory movement of the heart in relation to the stable bone position, wherein said drive unit is adapted to allow a movement to compensate for the respiratory movement in relation between said heart contacting organ and said bone.

Said respiration movement compensator may comprise a hydraulic, mechanical or pneumatic construction or a combination thereof, for to compensate for the respiratory movement.

The respiration movement compensator may comprise at least one of; a suspension involving a compressible cuff of air, for to compensate for the respiratory movement, a spring suspension, for to compensate for the respiratory movement and a guided movement using only frictional resistance, for to compensate for the respiratory movement.

In yet another embodiment the drive unit is adapted to be placed at least partly in the abdomen allowing the heart contacting organ to reach the heart, for creating said kinetic movement of the heart contacting organ, wherein preferable said drive unit is adapted to entering from the abdomen through the diaphragm muscle.

In another embodiment said fixation device is adapted to be mounted on the outside of the sternum, wherein said drive unit comprising an arm for passing subcutaneously from the outside of the sternum into the abdomen adapted to hold the drive unit, wherein said drive unit entering through the diaphragm muscle holding said heart contacting organ.

In another embodiment said drive unit further comprising a fibrotic tissue movement structure adapted to allow the respiratory movement of the heart in relation to the stable bone position, without interference from surrounding fibrotic tissue, when implanted in the body.

The fibrotic tissue movement structure may comprise a bellow allowing movement without stretching surrounding fibrosis, when implanted.

In yet another embodiment the heart contacting organ can change from exerting force to a first area of the heart to exerting force to a second area of the heart, after said implantable device has been implanted in said human patient, wherein said at least one heart contacting organ preferable comprises at least one hydraulic or pneumatic cushion.

In another embodiment the heart contacting organ further comprises a mechanical element, adapted to be movable to change the position of said force exerted on the heart of the human heart after said implantable device has been implanted in the human patient.

The implantable device may include a plate, and wherein said at least one hydraulic or pneumatic cushion is placed in connection to said plate, and wherein said plate enables movement of said cushion in relation to said plate to change the position of said hydraulic or pneumatic cushion and thereby change the position of said force exerted on the heart of the human patient after said implantable device has been implanted in the human patient.

The heart assistant device may be adapted to; pass through a laparoscopic trocar in the patient's body and/or pass through an opening in the diaphragm muscle from the abdominal side.

Preferable said drive unit is adapted to supply wireless or magnetic energy and said heart assistant device adapted to receive said wireless or magnetic energy to cause movements of said heart assistant device.

The heart assistant device may include an energy receiver or energy source adapted to be placed in the abdomen.

The heart assistant device preferable, comprising an electric wire adapted to connect said heart assistant device or drive unit to an internal energy source, said wire adapted to pass into the right atrium of the heart and further up in the venous blood vessel system, exiting the blood vessel system in or closer to the subcutaneous area, wherein said internal energy source is adapted to be connected to said wire via the subcutaneous area.

The heart assistant device could comprise; an internal control unit, a sensor sensing physiological electrical pulses or muscle contractions of the heart, wherein the control unit controls said heart assistant device according to the sensed information.

The heart assistant could further comprise an internal energy source, comprising an internal control unit adapted to transmit energy pulses to said electrode for achieving heart muscle contractions and controlling heart contractions, wherein said control unit is adapted to coordinate the heart assistant device with the heart contractions.

An object is to provide a device and a method for sturdy fixation of a heart help device. A sturdy and secure fixation will alleviate the heart from the weight of that of the heart pump device, driving members, energizing units and control logic.

According to one embodiment an implantable heart help device adapted for implantation in a human patient is provided. The implantable heart help device comprises a fixating member adapted to fixate said device to a part of the human body comprising bone.

According to one embodiment the implantable heart help device further comprises a second fixating member.

According to one embodiment the at least one fixating member comprises at least one plate adapted be fixated to the sternum. It is also conceivable that the first fixating member comprises a first plate adapted be fixated to the sternum, and the second fixating member comprises a second plate adapted be fixated to the sternum.

According to one embodiment the first fixating member is located on the anterior side of the sternum, and the second fixating member is located on the posterior side of the sternum.

According to one embodiment the first plate is adapted to be fixated to the anterior side of the sternum, and the second fixating member is adapted to be fixated to the posterior side of the sternum.

According to one embodiment the first plate is adapted to be fixated to said second plate using at least one screw.

According to one embodiment the at least one fixating member is adapted to be fixated to the cortex of the sternum of said human patient.

According to one embodiment the at least one fixating member is adapted to be fixated to the sternum of the human patient using at least one screw.

According to one embodiment the fixating member is contact with at least one arm, which in turn could operable.

According to one embodiment the fixating member is adapted to fixate the device to the anterior side of said sternum. The fixation could be done using at least one screw which could be place in the anterior cortex of the sternum. However it is also conceivable that said at least one screw is adapted to fixate said device to both the anterior cortex and the posterior cortex of said sternum.

According to one embodiment the at least one fixating member is adapted to fixate said device to the posterior side of said sternum. The fixation could be done using at least one screw which could be place in the posterior cortex of the sternum. However it is also conceivable that said at least one fixating member is adapted to fixate said device to both the posterior and the anterior cortex of said sternum.

According to one embodiment the fixating member is adapted to fixate said implantable heart help device to the sternum in more than one point.

According to one embodiment the fixating member is adapted to fixate said implantable heart help device using at least one screw and/or at least one pop-rivet and/or at least one through-going arrangement.

### Material

According to one embodiment the fixating member could comprise ceramic material, stainless steel and/or titanium.

### Pressing Position

According to one embodiment the second fixating member is displaceable in relation to the first fixating member.

According to one embodiment the first or second fixating member comprises at least one displaceable part for calibrating the location of the applied external force to the heart of a human patient.

According to one embodiment the second fixating member is displaceable in relation to the first fixating member for changing the position of the implantable heart help device.

According to one embodiment, the device comprises at least two displaceable members, the first member could comprise coils and the second member could comprise magnets.

According to one embodiment, the device comprises at least two displaceable members, the first member could comprise coils and the second member comprise magnets.

According to one embodiment the movement is created through successive energizing of the coils.

According to one embodiment the second fixating member is displaceable in three dimensions.

According to one embodiment the second fixating member comprises a locking function adapted to lock the second fixating member in a position.

According to one embodiment at least one displaceable member comprise at least one operable joint.

According to one embodiment the heart help device is adapted to exert an external force on the left ventricle.

According to one embodiment the heart help device is adapted to exert an external force on two different sides of the left ventricle.

According to one embodiment the heart help device is adapted to exert an external force on the right ventricle.

According to one embodiment the heart help device is adapted to exert an external force on two different sides of the right ventricle.

According to one embodiment the device is movable to change the position of the external force exerted on the heart.

According to one embodiment the second fixating member is operable using an implantable motor.

According to one embodiment the motor is an electrical motor.

According to one embodiment the motor is a servo motor.

According to one embodiment the motor is a hydraulic motor.

According to one embodiment the motor is a pneumatic motor.

According to one embodiment the second fixating member is operable from outside of the human body.

### The Heart Help Device

According to one embodiment the implantable heart help device exerts force on the outside of the heart muscle.

According to one embodiment, the implantable heart help device is an implantable device for improving the pump function of the heart of a human patient by applying an external force on the heart muscle, said device comprising at least one pump device comprising:
a rotating member having a rotating center,
a driving member attached to the rotating member and adapted to perform an eccentric movement in relation to the rotating center of the rotating member,
the driving member direct or indirect being in contact with a heart contacting organ, and wherein
the rotating movement of the rotating member is arranged to provide the driving member to exert an external force on the heart muscle.

According to one embodiment the driving member is adapted transport said eccentric movement from the rotating member to the heart contacting organ.

According to one embodiment the driving members is adapted to transport the eccentric movement from the rotating member to the heart contacting organ using at least one element selected from a group consisting of: wire, chain, belt, rod, shaft and flexible shaft.

According to one embodiment the rotating member is adapted for continuous movement.

According to one embodiment the implantable heart help device is an implantable device for improving the pump function of the heart of a human patient by applying an external force on the heart muscle, said device comprising at least one pump device comprising:
a first part having a first surface, and
a second part having a second surface, wherein
the first part is displaceable in relation to the second part,
the first and second surfaces abut each other, at least partially, and
the second part exerts, directly or indirectly, force on an external part of the heart muscle.

According to one embodiment the medical device further comprises a heart contacting organ in direct contact with said heart and in direct or indirect contact with at least one of: the first part and the second part.

According to one embodiment the first surface is substantially parallel to the second surface.

According to one embodiment the first part comprises coils and the second part comprises magnets.

According to one embodiment a movement is created through successive energizing of said coils.

According to one embodiment the first part is rotationally movable in relation to the second part.

According to one embodiment the first part is rotationally movable in relation to the second part.

According to one embodiment said first part is reciprocally movable in relation to said second part.

According to one embodiment the implantable heart help device is an implantable device for improving the pump function of the heart of a human patient by applying an external force on the heart muscle, said device comprising at least one pump device having a pump function comprising:
a piston adapted for reciprocating movement,
an operating device for operating the piston,
a heart contacting organ,
wherein the movement of the piston direct or indirect is
transported to said heart contacting organ to assist the pump function of the heart through said heart contacting organ.

According to one embodiment the piston is adapted for reciprocating movement using pressurized fluid in both movement directions.

According to one embodiment, further comprising two pressurized chambers, the first chamber adapted to have a high pressure and the second chamber having a low pressure. Said piston is adapted to use the large pressure chamber for moving said piston in both directions with pressurized high pressure fluid, further adapted to use the low pressure chamber for emptying the opposite side of the piston, when moved by the high pressure fluid, and further comprising a valve system to direct the low and high pressure chambers respectively to the right side of said piston.

According to one embodiment the piston comprises said heart contacting organ.

According to one embodiment the piston is arranged in a sleeve.

Drive According to one embodiment the pump device is operated by pressurized fluid in one direction and by vacuum in the opposite direction.

According to one embodiment, further comprises a pressurized fluid system.

According to one embodiment the pressurized fluid system further comprises a valve system.

According to one embodiment the pressurized fluid system further comprises a pressurized chamber.

According to one embodiment the pressurized fluid presses the piston adapted for reciprocating movement so that the piston affects the heart contacting organ.

According to one embodiment the pressurized fluid system a support pump, operated magnets and coils, which supplies a magnetic motor.

According to one embodiment the magnetic motor is operated by successive energizing of coils in connection with magnets.

According to one embodiment the implantable heart help device is an implantable device for improving the pump function of the heart of a human patient by applying an external force on the heart muscle, said implantable device comprising at least one implantable pump device comprising:
a fluid,
a first reservoir having a first volume and at least one movable wall portion, for varying said first volume, and
a second reservoir being in fluid connection with said first reservoir, wherein
said implantable pump device is adapted to allow free flow of fluid between said first reservoir and said second reservoir, and wherein
the first reservoir, the second reservoir and the fluid connection forms a fully implantable closed pump device, and wherein
the fully implantable closed pump device is adapted to transfer the first fluid volume and thereby transfer force from the first reservoir to the second reservoir, wherein the volume change in the second reservoir is adapted to directly or indirectly affect the heart muscle, and
an implantable operating device directly or indirectly driven by wireless energy for operating the movable wall to displace the fluid between the first and second reservoir, thereby affecting said heart muscle from the outside thereof.

According to one embodiment the implantable device further comprises a second implantable pump device comprising:
a fluid,
a first reservoir having a first volume and at least one movable wall portion, for varying said first volume, and
a second reservoir being in fluid connection with said first reservoir, wherein
said implantable pump device is adapted to allow free flow of fluid between said first reservoir and said second reservoir, and wherein
the first reservoir, said second reservoir and said fluid connection forms a fully implantable closed pump device, and wherein
the fully implantable closed pump device is adapted to transfer said first fluid volume and thereby transfer force from said first reservoir to the second reservoir wherein the volume change in the second reservoir is adapted to directly or indirectly affect the heart muscle, and
an implantable operating device directly or indirectly driven by wireless energy for operating the movable wall to displace the fluid between the first and second reservoir, thereby affecting said heart muscle from the outside thereof.

An implantable device for improving the pump function of the heart of a human patient by applying an external force on the heart muscle, said implantable device comprising at least one implantable pump device comprising:
a fluid,
a first reservoir having a first volume and at least one movable piston, for varying said first volume, and
a second reservoir being in fluid connection with said first reservoir, wherein
the implantable pump device is adapted to allow free flow of fluid between said first reservoir and said second reservoir, and wherein
the first reservoir, said second reservoir and said fluid connection forms a fully implantable closed pump device, and wherein
the fully implantable closed pump device is adapted to transfer the first fluid volume and thereby transfer force from the first reservoir to the second reservoir wherein the volume change in the second reservoir is adapted to directly or indirectly affect the heart muscle, and
an implantable operating device directly or indirectly driven by wireless energy for operating the movable wall to displace the fluid between the first and second reservoir, thereby affecting said heart muscle from the outside thereof.

According to one embodiment said implantable device further comprises a second implantable pump device comprising:
a fluid,
a first reservoir having a first volume and at least one movable piston, for varying said first volume, and
a second reservoir being in fluid connection with said first reservoir, wherein said implantable pump device is adapted to allow free flow of fluid between said first reservoir and said second reservoir, and wherein said first reservoir, said second reservoir and said fluid connection forms a fully implantable closed pump device, and wherein said fully implantable closed pump device is adapted to transfer said first fluid volume and thereby transfer force from said first reservoir to said second reservoir wherein the volume change in the second reservoir is adapted to directly or indirectly affect the heart muscle, and
an implantable operating device directly or indirectly driven by wireless energy for operating the piston to displace the fluid between the first and second reservoir, thereby affecting said heart muscle from the outside thereof.

According to one embodiment said implantable heart help device is an LVAD device.

According to one embodiment the implantable heart help device is an artificial heart device.

According to one embodiment the heart help device comprise ceramics.

According to one embodiment the heart help device comprise titanium.

According to one embodiment the heart help device comprise titanium.

### Method

In the preferred embodiment a method of surgically placing an active heart assistant device outside a patient's heart via a laparoscopic thoracic approach, the method comprising the steps of:
inserting a needle or a tube like instrument into the thorax of the patient's body,
using the needle or a tube like instrument to fill the thorax with gas thereby expanding the thoracic cavity,
placing at least two laparoscopic trocars in the patient's body,
inserting a camera through one of the laparoscopic trocars into the thorax,
inserting at least one dissecting tool through one of said at least two laparoscopic trocars and dissecting an intended placement area of the patient's heart,
placing the heart assistant device in the placement area in the thorax as one or more pieces comprising;
placing the heart contacting organ affecting the blood stream,
placing a drive unit creating kinetic movement to be used by the heart contacting organ,
mounting a fixation device in a stable position to human bone allowing said_drive unit and kinetic movement to get necessary contra force,
placing a respiration movement compensator for compensating for the respiratory movement of the heart in relation to the stable bone position, and
placing and connecting an implanted energy receiver or an internal source of energy for powering the heart assistant device to perform at least one of the following method steps;
at least partly compressing the heart and at least partly relaxing the heart assistant device to support the heart's pumping mechanism from the outside thereof.

In another embodiment an operation method for surgically placing an active heart assistant device in relation to a patient's heart, the method comprising the steps of
cutting the patient's skin,
opening the thoracic cavity,
dissecting a placement area where to place the heart assistant device inside in relation to the heart,
placing the heart assistant device in the placement area in the thorax as one or more pieces comprising;
placing the heart contacting organ affecting the blood stream,
placing a drive unit creating kinetic movement to be used by the heart contacting organ,
mounting a fixation device in a stable position to human bone allowing said_drive unit and kinetic movement to get necessary contra force,
placing a respiration movement compensator for compensating for the respiratory movement of the heart in relation to the stable bone position, and
placing and connecting an implanted energy receiver or an internal source of energy for powering the heart assistant device to perform at least one of the following method steps;
at least partly compressing the heart and at least partly relaxing the heart assistant device to support the heart's pumping mechanism from the outside thereof.

In yet another embodiment a method of surgically placing an active heart assistant device in relation to a patient's heart via a laparoscopic abdominal approach, the method comprising the steps of
inserting a needle or a tube like instrument into the abdomen of the patient's body,
using the needle or a tube like instrument to fill the abdomen with gas thereby expanding the abdominal cavity,
placing at least two laparoscopic trocars in the patient's abdomen
inserting a camera through one of the laparoscopic trocars into the abdomen,
inserting at least one dissecting tool through one of said at least two laparoscopic trocars and
dissecting and creating an opening in the diaphragm muscle,
dissecting an intended placement area of the patient's heart through said opening,
placing the heart assistant device in the placement area in the thorax as one or more pieces comprising;
placing the heart contacting organ affecting the blood stream,
placing a drive unit creating kinetic movement to be used by the heart contacting organ,
mounting a fixation device in a stable position to human bone allowing said_drive unit and kinetic movement to get necessary contra force,
placing a respiration movement compensator for compensating for the respiratory movement of the heart in relation to the stable bone position, and
placing and connecting an implanted energy receiver or an internal source of energy for powering the heart assistant device to perform at least one of the following method steps; at least partly compressing the heart and at least partly relaxing the heart assistant device to support the heart's pumping mechanism from the outside thereof.

Alternatively an operation method for surgically placing an active heart assistant device in relation to a patient's heart, the method comprising the steps of:
cutting the patient's skin,
opening the abdominal cavity,
dissecting and creating an opening in the diaphragm muscle,
dissecting a placement area where to place the heart assistant device through said opening,
placing the heart assistant device in the placement area in the thorax as one or more pieces comprising;
placing the heart contacting organ affecting the blood stream,
placing a drive unit creating kinetic movement to be used by the heart contacting organ,
mounting a fixation device in a stable position to human bone allowing said_drive unit and kinetic movement to get necessary contra force,
placing a respiration movement compensator for compensating for the respiratory movement of the heart in relation to the stable bone position, and
placing and connecting an implanted energy receiver or an internal source of energy for powering the heart assistant device to perform at least one of the following method steps; at least partly compressing the heart and at least partly relaxing the heart assistant device to support the heart's pumping mechanism from the outside thereof.

The four operation methods above, wherein the step of placing the heart assistant device additionally may comprise the step of:
supplying kinetic power from said drive unit to said heart assistant device causing movement of said heart contacting organ.

The four operation methods additionally may comprise the method step of:
connecting the drive unit with an implantable energy receiver or an internal energy source for powering said drive unit.

The operation method for surgically placing a heart assistant device in a patient's heart or blood vessel combining the methods with a thoraxial approach and an abdominal approach is a preferred embodiment.

The operation method, wherein the drive unit further comprising a stator and a rotor adapted to be driving at least a part of the heart assistant device with rotational energy is yet another alternative, the method further comprising the steps of
placing said stator and rotor in the abdomen or thorax, wherein said rotor is connecting to said heart assistant device,
supplying energy to said stator to rotate said rotor and thereby causing kinetic energy to be transported to said heart assistant device.

The operation method may comprise that an opening is performed from the abdomen through the thoracic diaphragm for placing the energy receiver or energy source in the abdomen.

The operation method, wherein said opening is performed in the thoracic diaphragm, is preferable positioned at the place where the pericardium is attached to the thoracic diaphragm.

In yet another method the heart assistant device or drive unit is using energy, direct or indirect, from an external energy source, supplying energy non-invasively, without any penetration through the patient's skin, for powering the heart assistant device or drive unit.

Alternatively said heart assistant device or drive unit is connected to an internal energy source via a cable, the method of placement further comprising;
dissecting and placing a wire connected to the heart assistant device or drive unit into the right atrium of the heart and further up in the venous blood vessel system,
exiting the blood vessel system in or closer to the subcutaneous area, such as in the vena subclavia, vena jugularis or vena brachialis placing an internal energy source in the subcutaneous area or close thereto or in the thorax or abdomen,
supplying from an external energy source energy non-invasively, without any penetration through the patient's skin, to power the internal energy source for indirect or direct power the heart assistant device or drive unit.

The operation method of placement may further comprise;
placing an electrode in the right atrium or ventricle of the heart
placing the wire to the electrode via the right atrium of the heart and further up in the venous blood vessel system,
exiting the blood vessel system in or closer to the subcutaneous area, such as in the vena subclavia, vena jugularis or vena brachialis,
placing an internal control unit in the subcutaneous area or close thereto or in the thorax or abdomen, the method further comprising at least one of the following steps;
transmitting energy pulses from said electrode for controlling heart contractions, and
coordinating the heart assistant device or drive unit.

In yet another embodiment the operation method of placement further comprising;
placing an electrode in the right atrium or ventricle of the heart
placing the wire to the electrode via the right atrium of the heart and further up in the venous blood vessel system,
exiting the blood vessel system in or closer to the subcutaneous area, such as in the vena subclavia, vena jugularis or vena brachialis,
placing an internal control unit in the subcutaneous area or close thereto or in the thorax or abdomen, the method further comprising at least one of the following steps;
receiving sensor input relating to electrical pulses or muscle contractions of the heart,
coordinating the heart assistant device or drive unit based on said sensor input.

A method of fixating an implantable heart help device in a human patient, for improving the pump function of the heart of a human patient by applying an external force on the heart muscle is further provided. The device comprises at least one heart contacting organ said device adapted to be fixated to the sternum of a human patient, said method comprising the steps of: cutting the skin of said human patient, dissecting an area of the sternum, fixating said implantable heart help device to said sternum, placing the movable heart contacting organ onto the heart of the patient, placing an operating device, operating said heart contacting organ to periodically exert force on the outside of said heart, withholding force from the sternum, mounted on said sternum, connecting a source of energy for powering said implantable device for improving the pump function of the heart.

According to one embodiment, the method further comprises the step of calibrating said implantable heart help device, so that the heart contacting organ is placed in an advantageous position.

According to one embodiment, the method further comprises the step of calibrating postoperatively non-invasively.

A surgical method related to placing an implantable device for improving the pump function of the heart of a human patient by applying an external force on the heart muscle is further provided. The device comprising at least one heart contacting organ said device adapted to be fixated to the sternum of a human patient, the method comprising the steps of: cutting the skin of said human patient, inserting a needle or a tube like instrument into the thorax of the patient's body, using the needle or a tube like instrument to fill the thorax with gas thereby expanding the thoracic cavity, placing at least two laparoscopic trocars in the patient's body, inserting a camera through one of the laparoscopic trocars into the thorax, inserting at least one dissecting tool through one of said at least two laparoscopic trocars and dissecting an intended placement area in the area of the sternum of the patient, fixating said implantable device onto the sternum with a first fixation device, placing the movable heart contacting organ onto the heart of the patient, placing an operating device, operating said heart contacting organ to periodically exert force on the outside of said heart, withholding force from the sternum, mounted on said sternum, connecting a source of energy for powering said implantable device for improving the pump function of the heart.

A surgical method of calibrating the position of a heart help device fixated to the sternum of a human patient is further provided, comprising the steps of: cutting the skin of the human body, and calibrating the position of said heart contacting organ.

According to one embodiment, the method further comprises the step of calibrating said implantable heart help device, so that the heart contacting organ is placed in an advantageous position.

According to one embodiment, the method further comprising the step of calibrating postoperatively non-invasively According to one embodiment the implantable heart help device is an implantable heart help device that exerts force on the outside of the heart muscle.

According to one embodiment the implantable heart help device is an implantable device for improving the pump function of the heart of a human patient by applying an external force on the heart muscle. The device comprising at least one pump device comprising: a rotating member having a rotating center, a driving member attached to said rotating member and adapted to perform an eccentric movement in relation to the rotating center of said rotating member. The driving member being in contact with a heart contacting organ, and wherein the rotating movement of the rotating member is arranged to provide the driving member to exert an external force on the heart muscle.

According to one embodiment the implantable heart help device is an implantable device for improving the pump function of the heart of a human patient by applying an external force on the heart muscle. The device comprising at least one pump device comprising: a first part having a first surface, and a second part having a second surface, wherein said first part is displaceable in relation to the second part. The first and second surfaces abut each other, at least partially, and said second part exerts, directly or indirectly, force on an external part of said heart muscle.

According to one embodiment the implantable heart help device is an implantable device for improving the pump function of the heart of a human patient by applying an external force on the heart muscle. The device comprising at least one pump device having a pump function comprising: a piston adapted for reciprocating movement, an operating device for operating the piston, a heart contacting organ. The movement of the piston assists the pump function of the heart through said heart contacting organ.

According to one embodiment the implantable heart help device is an implantable device for improving the pump function of the heart of a human patient by applying an external force on the heart muscle. The implantable device comprising at least one implantable pump device comprising: a fluid, a first reservoir having a first volume and at least one movable wall portion, for varying said first volume, and a second reservoir being in fluid connection with said first reservoir. The implantable pump device is adapted to allow free flow of fluid between said first reservoir and said second reservoir, and the first reservoir, the second reservoir and said fluid connection forms a fully implantable closed pump device. Furthermore the fully implantable closed pump device is adapted to transfer force from said first reservoir to said second reservoir.

According to one embodiment the implantable heart help device is an LVAD device.

According to one embodiment the implantable heart help device is an artificial heart device.

### Method

A method of fixating an implantable heart help device in a human patient is further provided. The method comprises the steps of: cutting the skin of said human patient, dissecting an area of the sternum, and fixating said implantable heart help device to the sternum. The method could further comprise the step of calibrating the implantable heart help device, so that it is placed in an advantageous position.

A surgical method of calibrating the position of a heart help device fixated to the sternum of a human patient is further provided. The method comprises the steps of: cutting the skin of said human patient, and calibrating the position of said heart help device.

A laparoscopic method of calibrating the position of a heart help device fixated to the sternum of a human patient is provided. The method comprising the steps of: making an incision in the skin of the human body shorter than 30 millimeters, and calibrating the position of said heart help device.

### Energizing

According to one embodiment of the present invention the device is a part of a system that may comprise a switch for manually and non-invasively controlling the device. The switch is according to one embodiment an electric switch and designed for subcutaneous implantation.

According to another embodiment the system further comprises a hydraulic device having a hydraulic reservoir, which is hydraulically connected to the device. The device could be manually regulated by pressing the hydraulic reservoir or automatically operated using a wireless remote control.

The wireless remote control system comprises, according to one embodiment, at least one external signal transmitter and an internal signal receiver implantable in the patient for receiving signals transmitted by the external signal transmitter. The system could operate using a frequency, amplitude, or phase modulated signal or a combination thereof.

According to one embodiment the wireless control signal comprises an analogue or a digital signal, or a combination of an analogue and digital signal. It is also conceivable that the signal comprises an electric or magnetic field, or a combined electric and magnetic field. According to another embodiment the wireless remote control further transmits a carrier signal for carrying the wireless control signal, said signal could comprise a digital, analogue or a combination of digital and analogue signals.

For supplying the system with energy it comprises, according to one embodiment, a wireless energy-transmission device for non-invasively energizing said device. According to said embodiment the energy-transmission device transmits energy by at least one wireless energy signal, which for example comprises a wave signal such as an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal.

It is further conceivable that the energy signal comprises an electric or magnetic field, or a combined electric and magnetic field, which can be transmitted using a carrier signal such as a digital, analogue or a combination of digital and analogue signals.

According to one embodiment the system further comprises an energy source for powering said device, which can be an implantable or external energy source or a combination thereof, in which case the internal and external energy sources can be in electric communication.

In an embodiment in which the system comprises an internal energy source, a sensor sensing a functional parameter correlated to the transfer of energy for charging the internal energy source may be provided, it is furthermore conceivable that a feedback device for sending feedback information from the inside to the outside of the patient's is provided.

According to another embodiment the system further comprises a sensor sensing a parameter such as a functional or physical parameter. Said functional parameter is, according to one embodiment, correlated to the transfer of energy for charging an internal energy source implantable in the patient. Said embodiment could furthermore comprise a feedback device for sending feedback information from inside to the outside of the patient's body and an implantable internal control unit for controlling the sensing. Above mentioned physical parameter could be one of body temperature, blood pressure, blood flow, heartbeats and breathing, and the sensor could be a pressure or motility sensor.

According to one embodiment the system could further comprise an external data communicator and an implantable internal data communicator communicating with the external data communicator, wherein the internal communicator feeds data related to said device or the patient to the external data communicator and/or the external data communicator feeds data to the internal data communicator. It is also conceivable that the system further comprises an operation device for operating said device, such as a motor or a pump, which can be electrically, hydraulically or pneumatically operated.

According to another embodiment the system has an energy-transmission device for transmitting wireless energy, wherein the wireless energy is used to directly power the operation device through for example creating kinetic energy for the operation of said device.

In embodiments where the system comprises an energy-transmission device for transmitting wireless energy, an energy-transforming device for transforming the wireless energy from a first form into a second form may be provided. Said energy-transforming device may directly power by the second form of energy. The energy could be in the form of a direct current or pulsating direct current, or a combination of a direct current and pulsating direct current, or an alternating current or a combination of a direct and alternating current, it is also conceivable that the energy is in the form of magnetic energy, kinetic energy, sound energy, chemical energy, radiant energy, electromagnetic energy, photo energy, nuclear energy or thermal energy. The system may further comprise an implantable accumulator for storing energy.

To prevent damage of the system it is conceivable that it comprises implantable electrical components including at least one voltage level guard and/or at least one constant current guard.

The invention also relates to a method of fixation of a heart help device wherein said fixation is achieved through the attaching of said heart help device to the sternum of a human patient.

### I: Male Sexual Impotence Treatment Prosthesis

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a male sexual impotence treatment prosthesis, examples of such male sexual impotence treatment prosthesis will now be described.

The present invention relates to a male sexual impotence treatment prosthesis apparatus, comprising an operable prosthesis implantable in the cavities of the corpora cavernosa of an impotent patient to provide erect penile condition, when the prosthesis is operated.

Male sexual impotence is a widespread problem. Many different solutions to this problem have been tried. In accordance with a prior system currently practiced a hydraulic inflatable/contractible silicon prosthesis is implanted in the cavities of the corpora cavernosa of the penis. In fluid connection with this prosthesis is a reservoir implanted retroperitonially and a pump therefore in the scrotum. By manually pumping the pump the prosthesis is filled with fluid from the reservoir to achieve erect penile condition or is emptied of fluid, which returns to the reservoir, to achieve flaccid penile condition. However, there are several more or less severe disadvantages of this solution. A problem that often occurs is that thick, hard fibrosis is created around the pump, which makes the system useless sooner or later.

Another solution to achieve erection is to restrict the blood flow leaving the penis. For example, U.S. Pat. Nos. 4,829,990, 4,958,630 and 5,048,511 disclose two hydraulically operated inflatable cuffs wrapped around the respective crura or penile exit veins. A disadvantage of such a solution is that it involves complicated surgery. U.S. Pat. No. 4,828,544 discloses another example on this solution, in which an artificial fistula system is surgically implanted and provides a primary fistula between the femoral artery and the femoral vein and a secondary fistula for leading blood from the primary fistula to the penis. An inflatable balloon engages the primary fistula between the secondary fistula and the vein. The balloon is in fluid connection with a manually compressible reservoir implanted in the scrotum. Again, implantation of this artificial fistula system requires delicate surgery.

Yet another solution is to inject a substance in the penile vein system to achieve erection. However, injections are painful and complicated for the patient.

Various impotence treatment devices in which fluid is distributed from a reservoir to an inflatable implanted prosthesis are disclosed in U.S. Pat. Nos. 3,855,122, 3,954,102, 4,009,711, 4,201,202, 4,235,227, 4,318,396 and 5,250,020.

U.S. Pat. No. 4,424,807 discloses another solution in which inflatable hydraulic cylindrical elements are implanted relatively deep into the corpus cavernosum.

The object of the present invention is to provide a new convenient male impotence prosthesis apparatus, which does not require manual manipulation of a combined reservoir/pump mechanism as in prior art placed in the scrotum of the patient, when the patient wants to achieve erection.

This object is obtained by an apparatus of the kind stated initially characterized by an energy transmission device for wireless transmission of energy from outside the patient's body to inside the patient's body for use in connection with the operation of the prosthesis, when the prosthesis is implanted.

As a result, the advantage is achieved that the male impotence prosthesis apparatus provides simple and effective energy transmission, which ensures an extended and reliable functionality of the apparatus, possibly for the rest of the patient's natural life, and at least many years.

Generally, the prosthesis is adapted to control the penis to change, preferably steplessly, between flaccid and erect penile condition. This gives the advantage that the patient is enabled to make fine adjustments of the prosthesis to achieve the desired erection without feeling pain.

Generally, the apparatus comprises an energy transforming device implantable in the patient for transforming the energy wirelessly transmitted by the energy transmission device from a first form into a second form, preferably different than the first form.

The energy transforming device may comprise at least one semiconductor type of component or a circuitry of such semiconductor components. The semiconductor component may comprise a transistor or microchip or similar electronic components. However, the semiconductor component may not comprise rectifying diodes.

In accordance with a main embodiment of the invention, the energy transforming device comprises at least one element having a positive region and a negative region and adapted to create an energy field between the positive and negative regions when exposed to the energy of the first form transmitted by the energy transmission device, so that the energy field provides the energy of the second form. Typically, the above-mentioned semiconductor component may include such an element.

In accordance with a preferred embodiment of the invention, the element comprises an electrical junction element capable of inducing an electric field between the positive and negative regions when exposed to the energy of the first form transmitted by the energy transmission device, whereby the energy of the second form comprises electric energy.

Consequently, the penile prosthesis suitably is electrically operated, whereby the positive and negative regions of the electrical junction element supply electric energy for the operation of the penile prosthesis. The apparatus suitably comprises implantable electric conductors connected to the positive and negative regions of the electrical junction element, whereby the electrical junction element is capable of supplying an electric current, such as a direct current, a pulsating direct current, a combination of a direct and pulsating direct current, an alternating current or a combination of a direct and alternating current, via the conductors. Furthermore, the electrical junction element may be capable of supplying a frequency, amplitude, or frequency and amplitude modulated analog, digital, or a combination of analog and digital signal, which is used in connection with control of the penile prosthesis.

The element, preferably in the form of an electrical semiconductor junction element, should be designed to generate an output current exceeding 1 μA when exposed to the energy of the first form transmitted by the energy transmission device. Suitably the electrical junction element forms a flat and thin sheet and has a volume of less than 2000 cm³ to be suited for subcutaneous implantation, so that the electrical junction element can be located just behind the skin of the patient. Alternatively, it would be possible to implant the element in the thorax or cephalic region of the patient, or in an orifice of the patient's body and under the mucosa or intraluminar outside the mucosa of the orifice. Of course, all the components of the energy transforming device including the electrical junction element in contact with the patient's body should be of biocompatible material.

For in vitro appliances, a particular type of an electrical semiconductor junction element has been commonly used, namely a so-called p-n (positive/negative) junction element, typically in the form of solar cells. A solar cell transfers solar energy in the form of visible light into electric energy in the form of direct current. For example, a p-n junction element may comprise two layers of semiconductor, one p-type (positive) and the other n-type (negative), sandwiched together to form a “p-n junction”. This p-n junction induces an electric field across the element when absorbing quanta of light (photons).

To be more precise, the quanta of light transfer their energy to some of the semiconductor's electrons, which are then able to move about through the material. For each such negatively charged electron, a corresponding positive charge—a “hole”—is created. In an ordinary semiconductor, these electrons and holes recombine after a short time and their energy is wasted as heat. However, when the electrons and holes are swept across the p-n junction in opposite directions by the action of the electric field, the separation of charge induces a voltage across the p-n junction element. By connecting the p-n junction element to an external circuit, the electrons are able to flow thereby creating a current.

Surprisingly, it has been proved that although both the skin and subcutis absorb energy from an external light beam directed against the skin portion behind which a properly designed p-n junction element is located, the light energy transmitted through the skin can induce a current from the p-n junction element strong enough (minimum 1 μA) to enable the operation of the electrically operated penile prosthesis. Thus, such a p-n junction element is now for the first time used for in vivo applications.

The apparatus may comprise an implantable pulse generator for generating electrical pulses from the energy of the second form produced by the energy field.

Generally, the energy transforming device is adapted to transform the energy of the first form directly or indirectly into the energy of the second form.

In accordance with a preferred embodiment of the invention, the energy of the second form comprises electric energy and the energy transforming device comprises a capacitor, which may be adapted to produce electric pulses from the transformed electric energy. Preferably, the capacitor may be adapted to produce the pulses as the energy transforming device transforms the energy of the first form transmitted by the energy transmission device into the electric energy of the second form. The capacitor should be small to facilitate implantation thereof; i.e. its capacity may not be more than 0.1 μF.

The apparatus may comprise an implantable stabilizer for stabilizing the energy of the second form. Where the energy of the second form comprises electric current the stabilizer may comprise at least one capacitor of the type described above.

In most embodiments of the invention, the apparatus comprises implantable electrical components. Where the electrical components include a capacitor of the type described above or an accumulator, at least one, preferably a single, voltage level guard may advantageously be provided, wherein the charge and discharge of the capacitor or accumulator is controlled by use of the voltage level guard. As a result, there is no need for any implanted current detector and/or charge level detector for the control of the capacitor, which makes the apparatus simple and reliable.

In a particular embodiment of the invention, the wireless energy of the first form comprises sound waves and the energy of the second form comprises electric energy, wherein the energy transforming device is adapted to directly transform the for example sound waves into electric energy.

It should be understood that the energy consuming parts of the apparatus for example a motor or pump may be or may not be energized with the unchanged wirelessly transmitted energy as this being transmitted as well as being or not being energized with energy different than the transmitted energy for example transformed into electrical energy but still directly used for energizing the energy consuming parts of the apparatus as the transmitted energy is transmitted. Alternatively the energy consuming parts of the apparatus may be energized from a implanted source of energy or storage device, which still may be loaded with wireless energy. In all these aspects it is preferable to be able to wirelessly control the release of energy and get an feedback of the result of the performed function of the device. Direct use of transmitted energy may be unreliable without a feedback what has happened, has the energy reached its goal?

The apparatus may comprise an implantable motor or pump for operating the penile prosthesis, wherein the motor or pump is powered by the transformed energy.

In accordance with a main aspect of the invention, the energy transmission device may be adapted to transmit wireless energy for direct use in connection with the operation of the penile prosthesis, as the wireless energy is being transmitted. The advantage of directly using energy as it is transmitted is that the apparatus can be of a very simple design and the few components involved makes the apparatus extremely reliable. For example, the energy transmission device may be adapted to directly power the motor or pump with wireless energy. The wireless energy may comprise a magnetic field or electromagnetic waves, suitably in the form of a signal, for direct power of the motor or pump. All the various functions of the motor and associated components described in the present specification may be used where applicable.

As an alternative to the above-noted main aspect of the invention, the energy transforming device may be adapted to supply the energy of the second form for direct use in connection with the operation of the penile prosthesis, as the energy of the first form is being transformed into the energy of the second form. Consequently, the energy transforming device may be adapted to directly power the motor or pump with the energy of the second form.

Generally, the energy transforming device directly operates the penile prosthesis with the energy of the second form in a non-magnetic, non-thermal or non-mechanical manner.

Where the apparatus comprises a motor, which may be adapted to directly or intermittently operate the penile prosthesis, the energy transforming device may power the motor with the energy of the second form. Suitably, the penile prosthesis is operable to perform a reversible function and the motor is capable of reversing said function.

In accordance with another embodiment of the invention, the penile prosthesis comprises a hydraulic penile prosthesis, and the apparatus comprises an implantable pump for operating the hydraulic penile prosthesis, wherein the energy transforming device supplies the energy of the second form for driving the pump. Preferably, the pump is not a plunger type of pump, but may comprise a peristaltic or membrane pump.

The energy transforming device preferably is capable of generating as the energy of the second form a current exceeding 1 μA, when transferring the energy of the first form transmitted by the energy transmission device.

The apparatus may comprise an implantable adjustment device for adjusting the penile prosthesis to change between erect and flaccid penile states. In accordance with a first alternative the adjustment device is adapted to mechanically adjust the penile prosthesis. In accordance with a second alternative the adjustment device is adapted to hydraulically adjust the penile prosthesis by using implanted hydraulic means. Such hydraulic means may not use hydraulic fluid of the kind having a viscosity that substantially increases when exposed to heat or a magnetic field.

The apparatus of the present invention is not limited to the use of visible light for the wireless transmission of energy. Thus, in accordance with a broad aspect of the invention, the energy transmission device transmits energy by at least one wireless signal, preferably containing radiant energy.

The wireless signal may comprises a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined. Alternatively, the wave signal may comprise a sound wave signal, such as an ultrasonic signal. Generally, the wireless signal may comprise a digital, analog or a digital and analog signal.

The energy of the first form transmitted by the energy transmission device may comprise an electric or magnetic field transmitted in pulses, for example digital pulses. Furthermore, the energy transforming device may transform the energy of the first form, which may comprise polarized energy, into a direct current, pulsating direct current, a combination of a direct and pulsating direct current, an alternating current or a combination of a direct and alternating current. Alternatively, the energy of the first form may comprise kinetic energy.

The energy of the second form may comprise a frequency, amplitude or frequency and amplitude modulated analog, digital or combined analog and digital signal.

The penile prosthesis may be non-inflatable, i.e. with no hydraulic fluid involved for the adjustments of the penile prosthesis. This eliminates problems with fluid leaking from the penile prosthesis.

The apparatus suitably comprises implantable electric conductors connected to the energy transforming device, whereby the energy transforming device is capable of supplying an electric current, such as direct current, a pulsating direct current, a combination of a direct and pulsating direct current, an alternating current or a combination of a direct and alternating current, via the conductors. Furthermore, the energy transforming device may be capable of supplying a frequency, amplitude, or frequency and amplitude modulated analog, digital, or a combination of analog and digital signal, which is used in connection with control of the penile prosthesis.

In accordance with a main embodiment of the invention, the apparatus comprises an implantable operation device for operating the penile prosthesis and a control device for controlling the operation device, wherein the energy transforming device powers the operation device with the energy of the second form. The operation device preferably comprises a motor, for example an electric linear motor or an electric rotary motor that is controlled by the control device to rotate a desired number of revolutions.

Optionally, an implantable gearing may be connected to the motor. The electric motor may have electrically conductive parts made of plastics. Alternatively, the motor may comprise a hydraulic or pneumatic fluid motor, wherein the control device controls the fluid flow through the fluid motor. Motors currently available on the market are getting smaller and smaller. Furthermore, there is a great variety of control methods and miniaturized control equipment available. For example, the number of revolutions of a rotary motor may be analyzed by a Hall-element just a few mm in size.

In accordance with another embodiment of the invention, the penile prosthesis comprises hydraulic means and the operation device is adapted to conduct a hydraulic fluid in the hydraulic means. The operation device comprises a fluid conduit connected to the hydraulic means of the penile prosthesis, and a reservoir for fluid, wherein the reservoir forms part of the conduit. The reservoir may form a fluid chamber with a variable volume, and the operation device may be adapted to distribute fluid from the chamber to the hydraulic means of the penile prosthesis by reduction of the volume of the chamber and to withdraw fluid from the hydraulic means to the chamber by expansion of the volume of the chamber. The operation device suitably comprises an implantable motor used for reducing and expanding the volume of the chamber. Also, the operation device may comprise a pump for pumping the hydraulic fluid in the hydraulic means of the penile prosthesis. All of the hydraulic components involved are preferably devoid of any non-return valve. This is of great advantage, because with valves involved there is always a risk of malfunction due to improperly working valves.

The control device may be adapted to reverse the operation device by shifting polarity of the energy of the second form. Where the operation device comprises an electric motor the energy of the second form suitably comprises electric energy.

In accordance with yet another embodiment of the invention, the penile prosthesis is operable to perform a reversible function, such as erecting the penis and then reverse by making the penis flaccid, and there is a reversing device implanted in the patient for reversing the function performed by the penile prosthesis. Such a reversing function preferably involves erecting the penis and making the penis flaccid by the penile prosthesis, suitably in a stepless manner. In this connection, the control device suitably controls the reversing device, which may include a switch, to reverse the function performed by the penile prosthesis. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch it may be operable by the energy of the second form. In this case, the control device suitably controls the operation of the switch by shifting polarity of the energy of the second form supplied to the switch. The switch may comprise an electric switch and the source of energy may supply electric energy for the operation of the switch.

In accordance with an advantageous embodiment of the invention, the apparatus further comprises an energy storage device implanted in the patient for storing the energy of the second form and for supplying energy in connection with the operation of the penile prosthesis. The implanted energy storage device preferably comprises an electric source of energy, such as an accumulator, a rechargeable battery or a combination of an accumulator and rechargeable battery.

The apparatus may further comprise a switch implantable in the patient for switching the operation of the penile prosthesis and a source of energy implantable in the patient. Such a source of energy preferably is a battery. Alternatively, the source of energy is an accumulator that also may store the energy of the second form.

In accordance with a first alternative, the switch is operated by the energy of the second form supplied by the energy storage device to switch from an off mode, in which the source of energy is not in use, to an on mode, in which the source of energy supplies energy for the operation of the penile prosthesis. In this case, the implanted source of energy may comprise a battery, preferably having a lifetime of at least 10 years, or an accumulator. However, other kinds of sources are also conceivable, such as a nuclear source of energy or a chemical source of energy (fuel cells).

In accordance with a second alternative, the apparatus further comprises a remote control for controlling the supply of energy of the implanted source of energy, wherein the switch is operated by the energy of the second form supplied by the energy storage device to switch from an off mode, in which the remote control is prevented from controlling the source of energy and the source of energy is not in use, to a standby mode, in which the remote control is permitted to control the source of energy to supply energy for the operation of the penile prosthesis.

In accordance with a third alternative, the energy storage device is omitted, wherein the switch is operated by the energy of the second form supplied by the energy transforming device to switch from an off mode, in which the remote control is prevented from controlling the source of energy and the source of energy is not in use, to a standby mode, in which the remote control is permitted to control the source of energy to supply energy for the operation of the penile prosthesis.

In accordance with a fourth alternative, also the remote control is omitted, wherein the switch is operated by the energy of the second form supplied by the energy transforming device to switch from an off mode, in which the source of energy is not in use, to an on mode, in which the source of energy supplies energy for the operation of the penile prosthesis. Where applicable, in the described embodiments the switch may switch when the energy transmission device is transmitting wireless energy, preferably while the transferred energy of the second form is stabilized by an implanted capacitor, which may temporarily (for a few seconds) store the energy of the second form.

In the above noted third and fourth alternatives, the energy transmission device may be substituted for the energy transforming device, whereby the switch is operated by the energy of the first form.

The switch mentioned above may comprise an electronic switch or, where applicable, a mechanical switch.

The advantage of using a switch above all is increased control safety; i.e. interfering signals in the patient's surroundings cannot affect the implanted penile prosthesis. Furthermore, the lifetime of the implanted source of energy will be significantly prolonged, since the energy consumption of the apparatus will be reduced to a minimum. During the above-mentioned standby mode, the remote control uses energy from the implanted source of energy. By means of the energy transmission device energy may be transmitted to activate the switch to connect the implanted source of energy only when energy is required in connection with the operation of the penile prosthesis.

All of the above embodiments may be combined with at least one implantable sensor for sensing at least one physical parameter of the patient, wherein the control device may control the penile prosthesis in response to signals from the sensor. For example, the sensor may sense ejaculation or comprise a pressure sensor for directly or indirectly sensing the pressure in the urethra. The control device may comprise an internal control unit implanted in the patient for, preferably directly, controlling the penile prosthesis in response to signals from the sensor. In response to signals from the sensor, for example ejaculation, pressure, or any other important physical parameter, the internal control unit may send information thereon to outside the patient's body. The control unit may also automatically control the penile prosthesis in response to signals from the sensor. For example, when the penis is in erect state the control unit may control the penile prosthesis to make the penis flaccid in response to the sensor sensing an abnormally high pressure against the penile prosthesis.

Alternatively, the control device may comprise an external control unit outside the patient's body for, suitably directly, controlling the penile prosthesis in response to signals from the sensor. The external control unit may store information on the physical parameter sensed by the sensor and may be manually operated to control the penile prosthesis based on the stored information. In addition, there may be at least one implantable sender for sending information on the physical parameter sensed by the sensor.

An external data communicator may be provided outside the patient's body and an internal data communicator may be implanted in the patient for communicating with the external communicator. The internal communicator may feed data related to the patient, or related to the penile prosthesis, back to the external communicator. Alternatively or in combination, the external communicator may feed data to the internal communicator. The internal communicator may suitably feed data related to at least one physical signal of the patient.

The apparatus may further comprise an implantable programmable control unit for controlling the penile prosthesis, preferably over time in accordance with an activity schedule program. This will advance the apparatus and make possible an adaptation of the apparatus to the individual patients.

Many of the above embodiments are suitably remote controlled. Thus, the apparatus advantageously comprises a wireless remote control transmitting at least one wireless control signal for controlling the penile prosthesis. With such a remote control it will be possible to adapt the function of the apparatus to the patient's need. The control signal may comprise a frequency, amplitude or frequency or amplitude modulated signal. Furthermore, the control signal may comprise an analogue or a digital signal, or a combination of an analogue and digital signal.

The wireless remote control may be capable of obtaining information on the condition of the implanted penile prosthesis and of controlling the penile prosthesis in response to the information. Also, the remote control may be capable of sending information related to the penile prosthesis from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient.

The wireless remote control may transmit a carrier signal for carrying the control signal, wherein the carrier signal is frequency, amplitude or frequency and amplitude modulated. The carrier signal may also comprise digital, analog or a combination of digital and analog signals. Such signals may comprise wave signals. Also the control signal used with the carrier signal may be frequency, amplitude or frequency and amplitude modulated, and be digital, analog or combined digital and analog.

The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

The energy transmission device may function different from or similar to the energy transforming device. For example, the energy transmission and transforming devices function differently when the energy transmission device comprises a coil used for transmitting the energy of the first form and the energy transforming device comprises an electrical junction element for transforming the transmitted energy into the energy of the second form. The energy transmission and transforming devices function similar to each other when the energy transmission device comprises a coil used for transmitting the energy of the first form and the energy transforming device also comprises a coil for transforming the transmitted energy into the energy of the second form.

In accordance with an alternative embodiment of the invention, the apparatus comprises an activatable source of energy implantable in the patient, wherein the source of energy is activated by wireless energy transmitted by the energy transmission device, to supply energy which is used in connection with the operation of the penile prosthesis.

The implantable penile prosthesis suitably is embedded in a soft or gel-like material. For example, a silicone material having hardness less than 20 Shore.

Preferable the present invention provides an a male sexual impotence treatment prosthesis, comprising an prosthesis device implanted in the corpora cavernosa of the patients penis, who suffers from impotence and an adjustment device which temporarily achieve an erected status of the penis and an powered operation device which is able to perform a reversible function to adjust said adjustment device.

In another embodiment the male sexual impotence treatment apparatus, comprising a hydraulic adjustment device, and further comprising a reservoir implantable in the patient and containing hydraulic fluid, and a conduit providing fluid connection between the reservoir and the hydraulic adjustment device, characterized in that the operation device being adapted to operate the hydraulic adjustment device by distributing hydraulic fluid through the conduit between the reservoir and the hydraulic adjustment device, the conduit and hydraulic adjustment device being devoid of any non-return valve to permit free flow of hydraulic fluid in both directions in the conduit.

Another alternative is a pump pumping in only one direction and an adjustable valve to change the direction of fluid to either increase or decrease the amount of fluid in the reservoir. This valve may be manipulated either manually, mechanically, magnetically, or hydraulically.

Alternatively, or in combination with a powered operation device, the servo means may be used, which enables manual manipulation without need for strong manipulation forces. The servo means may comprise hydraulic means, electric control means, magnetic means, or mechanical means, which may be activated by manual manipulating means. Using a servo system will save the use of force when adjusting the adjustment device, which may be of importance in many applications.

The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible and/or include a gearing. Where the operation device comprises a motor, the reversing device is adapted to reverse the motor.

The main embodiment described above including the reservoir may alternatively be equipped with a servo means comprising a reverse servo. The term “reverse servo” is to be understood as a mechanism that transfers a strong force acting on a moving element having a short stroke into a weak force acting on another moving element having a long stroke; i.e. the reverse function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device.

The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the normal servo means and the specific reverse servo may be used in connection with all of the various components and solutions described in the present specification.

Thus, the reverse servo may be adapted to provide relative displacement between the first and second wall portions of the reservoir, suitably in response to the pressure in the reservoir, in order to change the volume of the chamber of the reservoir.

Generally, the servo means, including the reverse servo, comprises a pressure controlled servo means. The alarm mentioned above may alternatively be adapted to generate an alarm signal in response to the lapse of a predetermined time period during which the pressure controlling the servo means exceeds a predetermined high value.

The reverse servo may comprise magnetic means, electric means or manual manipulation means or a combination thereof. Preferably, however, the reverse servo comprises hydraulic means.

In accordance with a particular embodiment of the invention, the reverse servo further comprises a servo reservoir defining a chamber containing servo fluid, and the operation device comprise first and second wall portions of the servo reservoir, which are displaceable relative to each other to change the volume of the chamber of the servo reservoir. The first and second wall portions of the servo reservoir may be displaceable relative to each other by magnetic means, hydraulic means, or electric control means.

Where the reverse servo comprises hydraulic means it may further comprise a fluid supply reservoir connected to the servo reservoir in a closed system and containing a further predetermined amount of fluid. The fluid supply reservoir defines a chamber for the further predetermined amount of fluid and the operation device is adapted to change the volume of the chamber and thereby control the amount of fluid in the servo reservoir. The fluid supply reservoir comprises first and second wall portions, which are displaceable relative to each other to change the volume of the chamber of the fluid supply reservoir. Suitable, the fluid supply reservoir increases the amount of fluid in the servo reservoir in response to a predetermined first displacement of the first wall portion of the fluid supply reservoir relative to the second wall portion of the fluid supply reservoir and decreases the amount of fluid in the servo reservoir in response to a predetermined second displacement of the first wall portion of the fluid supply reservoir relative to the second wall portion of the fluid supply reservoir.

All the above described various components, such as the motor, pump and capacitor, may be combined in the different embodiments where applicable. Also the various functions described in connection with the above embodiments may be used in different applications, where applicable.

All the various ways of transferring, transforming and controlling energy presented in the present specification may be practiced by using all of the various components and solutions described.

The present invention also provides a method for implanting the male impotence prosthesis apparatus described above comprising the steps of cutting an opening in an impotent patient's mucosa in an orifice of the patient's body, and implanting the energy transforming device of the apparatus in the patient's body through the opening. Alternatively, the cutting step may comprise cutting an opening in the patient's skin and the implanting step may comprise implanting the energy transforming device in the patient's body through the opening.

There is also provided a laparoscopical implanting method, in accordance with a first alternative, comprising the steps of providing the male impotence prosthesis apparatus described above, placing at least two laparoscopic cannula within an impotent patient's body, and implanting the energy transforming device of the apparatus in the patient's body by using the at least two laparoscopic cannula.

### J: Affecting an Aneurysm on a Blood Vessel

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for affecting an aneurysm on a blood vessel, examples of such devices for affecting an aneurysm on a blood vessel will now be described.

It is an object of the present invention to overcome or at least reduce some of the problems associated with treatment and monitoring of an aneurysm.

This object and others are obtained by the method, system and device as set out in the appended claims. Thus, by providing a member around the aneurysm, the aneurysm can be treated and monitored.

In accordance with one embodiment a device comprising an implantable member adapted to hold fluid is provided. The member is adapted to be placed in connection with a blood vessel having the aneurysm and to exercise a pressure on the aneurysm. Hereby the aneurysm can be prevented from bursting.

In accordance with one embodiment the device is adapted to prevent or reduce an expansion of said aneurysm.

In accordance with one embodiment the device is adapted to be postoperatively adjusted.

In accordance with one embodiment the device is adapted to perform self-adjustments of the pressure applied onto said aneurysm within a predetermined treatment interval.

In accordance with one embodiment the device can also comprise a control unit and a sensor. The control unit is then adapted to control pressure adjustments of based on a signal generated by the sensor.

In accordance with one embodiment the device can also comprise a pressure regulator adapted to regulate the pressure in the member.

In accordance with one embodiment the implantable member can be Y-shaped and adapted to be placed at the Aorta Bifurcation In accordance with one embodiment the device can be configured to apply a pressure that is equal or less than the diastolic blood pressure of a treated patient.

In accordance with one embodiment the device can be adapted to increase the pressure on the blood vessel when the aneurysm expands.

In accordance with one embodiment the device can also comprise a sensor for sensing an expansion of the aneurysm.

In accordance with one embodiment the device can also comprise an electrical pulse generator adapted to provide electrical signals for stimulation of the aneurysm wall via electrodes located on the inside of the implantable member.

In a preferred embodiment, the system comprises at least one switch implantable in the patient for manually and non-invasively controlling the device.

In another preferred embodiment, the system comprises a wireless remote control for non-invasively controlling the device.

In a preferred embodiment, the system comprises a hydraulic operation device for operating the device.

In one embodiment, the system comprises comprising a motor or a pump for operating the device.

The invention also extends to methods for implanting the device and to a computer program product adapted to control the device.

Any feature in any of the four combinations of features in the combination embodiments described below may be used in any combination and furthermore in combination with any other feature or embodiment described in any of the other FIGURES or FIGURE text or descriptions in this application.

### First Combination Embodiments Includes Electrical Stimulation Comprising

A medical device including a stimulation device for treating a vascular aneurysm of a human or mammal patient comprising:
at least one implantable electrode adapted to placed in close connection to the aneurysm, the at least one electrode being adapted to provide an electrical stimulation pulse on a wall portion of the aneurysm.

At least one electrode is adapted to stimulate multiple stimulation points. Alternatively at least two electrodes are provided and wherein groups of stimulation points are controllable to be individually stimulated.

A pulse generator adapted to generate positive and negative electrical stimulation pulses.

Electrical stimulation pulses, which may have a constant current and preferable the stimulation device deliver the electrical stimulation pulse as pulse train stimulation with breaks to allow the vessel to rest.

A stimulation device that deliver the electrical stimulation pulses at different time intervals.

A device preferable delivering the electrical stimulation pulse as a pulse width modulated stimulation pulse.

A stimulation device preferable deliver the electrical stimulation pulse during the systolic phase.

A stimulation device further comprising a monitoring system for detecting an expansion of the aneurysm. Also to avoid any fast expansion and burst leading to death.

If so said monitoring system may increase intensity and or position of the stimulation, when detecting an expansion of the aneurysm.

A method of treating an aneurysm of a mammal patient by providing the medical device according to any feature disclosed herein, comprising the steps of:
inserting a needle or a tube like instrument into the patient's abdominal cavity,
using the needle or tube like instrument to fill a part of the patient's body with gas and thereby expanding said abdominal cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing said medical device, comprising a stimulation device, onto said the aneurysmic blood vessel, and
stimulating said aneurysm to increase the tonus of the aneurysm wall.

An alternative method of treating an aneurysm of a mammal patient by providing the medical device including any feature disclosed herein, comprising the steps of:
inserting a needle or a tube like instrument into the patient's thoraxial cavity,
using the needle or tube like instrument to fill a part of the patient's body with gas and thereby expanding said thoraxial cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing said medical device, comprising a stimulation device, onto said the aneurysmic blood vessel, and
stimulating said aneurysm to increase the tonus of the aneurysm wall.

An alternative method of treating an aneurysm of a mammal patient by providing the medical device including any feature disclosed herein, said method comprising the steps of:
cutting the skin in the abdominal or thoraxial wall of said mammal patient,
dissecting an area of the aneurysm,
placing said medical device, comprising a stimulation device, onto said aneurysm, and
stimulating said aneurysm to increase the tonus of the aneurysm wall.

A method of treating an aneurysm of a mammal patient by providing the medical device including any feature disclosed herein, said method comprising the steps of:
cutting the skin of said mammal patient,
dissecting an area of the aneurysm,
placing said medical device, comprising a stimulation device, onto said aneurysm, and
stimulating said aneurysm to increase the tonus of the aneurysm wall.

Additionally a computer program product comprising computer program segments that when executed on a computer causes the computer to generate a pattern of signals for an implantable electrode adapted to be placed in close connection to an aneurysm, the at least one electrode being adapted to provide an electrical stimulation pulse on a wall portion of the aneurysm.

A device including a digital storage medium comprising the computer program product.

### Second Combination Embodiments Includes a Hydraulic System Putting Pressure on the Aneurysm Comprising

A device for treating an aneurysm of a human or mammal patient comprising:
An implantable member adapted to hold fluid, wherein said member is adapted to be placed in connection with a blood vessel having the aneurysm, the member being adapted to exercise a pressure on the aneurysm of said blood vessel.

A device preferable adapted to prevent or reduce an expansion of said aneurysm.

A device adapted to be postoperatively adjusted. The device is normally non-invasively adjustable.

A device preferable adapted to perform self-adjustments of the pressure applied onto said aneurysm within a predetermined treatment interval.

A device normally comprising a control unit and a sensor, the control unit being adapted to control pressure adjustments of based on a signal generated by the sensor.

The sensor may comprise any type of sensor. Preferable a pressure regulator is adapted to regulate the pressure in the member, wherein the pressure regulator preferable is adapted to even out the difference in pressure in the implantable member during the systolic and diastolic phase for reducing the pressure difference or providing a substantially even outside pressure on the aneurysm. The pressure regulator may comprise pressure tank.

An implantable member which is alternatively Y-shaped, wherein the implantable Y-shaped member normally is adapted to be placed at the Aorta Bifurcation A pressure regulator in one embodiment comprises an expandable first reservoir.

The expandable first reservoir preferable is spring loaded.

A device wherein the pressure regulator in a preferred embodiment comprises a pump.

A device further comprising a second reservoir and a pump adapted to move liquid between the first and second reservoirs.

A device wherein preferable said first reservoir has a predetermined optimal pressure regulation volume treatment interval and wherein said pump is adapted to pump liquid from the first to the second reservoir to keep said first reservoir within said regulation interval, when said aneurysm expands and to pump liquid from said implantable member into said first reservoir.

A device preferable provides a pressure equal or less than the diastolic blood pressure of a treated patient.

A device preferable adapted to increase the pressure on the blood vessel when the aneurysm expands.

A device comprising a control device adapted to increase the pressure on the blood vessel when the aneurysm expands more than a predetermined value, preferable during a time period.

A control unit adapted to control the expansion of said aneurysm by controlling the pressure applied on the blood vessel when the aneurysm expands.

A device preferable further comprising a sensor for sensing an expansion of the aneurysm.

A device preferable further comprising a volume control unit adapted to directly or indirectly control the volume in the implantable member based on a signal generated by the sensor for controlling an expansion of the aneurysm, wherein normally said volume control unit controls the volume in the implantable member based on a signal indicative of: flow of fluid from said implantable member or pressure in said fluid filled in said implantable member.

A device wherein the implantable member is divided into a plurality of sub-reservoirs.

A device wherein the sub-reservoirs are provided axially along the blood vessel or radially along the blood vessel.

A device wherein preferable at least one reservoir is located above said aneurysm and one reservoir is located below said aneurysm.

A device further comprising a logic circuitry for determining when the aneurysm is expanding based on the signal from the sensor.

A device further comprising an electrical pulse generator adapted to provide electrical signals for stimulation of the aneurysm wall via electrodes located on the inside of the implantable member.

A control unit adapted to vary to position of the electrical stimulation signals for stimulation of the aneurysm.

A method of treating an aneurysm of a mammal patient by providing the medical device according to any feature disclosed herein, comprising the steps of:
inserting a needle or a tube like instrument into the patient's abdominal cavity,
using the needle or tube like instrument to fill a part of the patient's body with gas and thereby expanding said abdominal cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and adjusting the pressure the device exerts onto said aneurysm.

An alternative method of treating an aneurysm of a mammal patient by providing the medical device including any feature disclosed herein, comprising the steps of:
inserting a needle or a tube like instrument into the patient's thoraxial cavity,
using the needle or tube like instrument to fill a part of the patient's body with gas and thereby expanding said thoraxial cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and
adjusting the pressure said device exerts onto said aneurysm.

An alternative method of treating an aneurysm of a mammal patient by providing the medical device including any feature disclosed herein, said method comprising the steps of:
cutting the skin in the abdominal or thoraxial wall of said mammal patient,
dissecting an area of the aneurysm,
placing said device onto said aneurysm, and
starting the stimulation device and adapted to adjust any parameter related to said stimulation.
adjusting the pressure said device exerts onto said aneurysm.
adjusting the pressure said device exerts onto said aneurysm.

A computer program product comprising computer program segments that when executed on a computer causes the computer to control the pressure applied by an implantable member adapted to hold fluid and adapted to be placed in connection with a blood vessel having an aneurysm. A digital storage medium comprising the computer program product.

### Third Combination Embodiments Includes a Mechanical System Putting Pressure on the Aneurysm Including any Feature in any Combination, Comprising

A device for treating a vascular aneurysm of a human or mammal patient comprising:
An implantable member adapted to be placed in connection with a blood vessel having an aneurysm for providing a pressure from outside the blood vessel, the device being adapted to be adjusted postoperatively.

A device preferable adapted to prevent or reduce an expansion of said aneurysm.

A device adapted to monitor an expansion of said aneurysm.

The device is preferable adjustable non-invasively.

A device adapted to perform self-adjustments of the pressure applied onto said aneurysm within a predetermined treatment interval.

A device comprising an control unit and a sensor, wherein the control unit is adapted to control the pressure applied onto said aneurysm based on said signal generated by the sensor.

A device, wherein the surface of the member facing the blood vessel is adapted to exercise pressure on the blood vessel.

A device, wherein the pressure on the blood vessel is mechanically exercised.

A, wherein the mechanically exercised pressure is controlled hydraulically.

A device, wherein mechanical pressure on the blood vessel is directly or indirectly exercised by a motor or a pump.

A, wherein the implantable member is generally cylindrical A device, wherein the implantable member comprises a number of segments being individually adjustable.

A device, wherein the implantable member is a Y-shaped member

A device, wherein the implantable Y-shaped member is adapted to be placed at the Aorta Bifurcation.

A pressure regulating system adapted to even out the difference in pressure in the implantable reservoir in the systolic and diastolic phase to reduce the differences or to achieve a substantially even pressure affecting said aneurysm from the outside of said blood vessel.

A device, wherein the implantable member is an elastic member.

A device, wherein the elastic member is a band.

A device, wherein the elastic member is adapted to apply a pressure onto said aneurysm and has an expansion interval wherein the pressure applied is substantially constant or within an interval for treating and reducing expansion of the aneurysm.

A device, wherein the implantable member is spring loaded.

A device according to claim 1, wherein the implantable member is hydraulically operated.

A device, wherein the implantable member is pneumatically operated

A device, wherein the implantable member is adapted to exert an essentially constant pressure or a pressure reducing the pressure difference, caused by the changes in blood pressure in said blood vessel, on the aneurysm.

A device, wherein the provided pressure is equal or less than the diastolic blood pressure of a treated patient.

A device further comprising a control unit adapted to increase the pressure on the blood vessel when the aneurysm expands.

A device comprising a control device adapted to increase the pressure on the blood vessel when the aneurysm expands more than a predetermined value.

A device comprising a control device adapted to increase the pressure on the blood vessel when the aneurysm expands more than a predetermined value during a time period.

A device, further comprising a sensor or a measuring device for sensing an expansion of the aneurysm.

A device, further comprising logic circuitry for determining when the aneurysm is expanding based on a signal from a sensor or measuring device.

A device, further comprising an electrical pulse generator adapted to provide stimulation of the aneurysm wall via electrodes located on the inside of the implantable member.

A method of treating an aneurysm of a mammal patient by providing the medical device according to any feature disclosed herein, comprising the steps of
inserting a needle or a tube like instrument into the patient's abdominal cavity,
using the needle or tube like instrument to fill a part of the patient's body with gas and thereby expanding said abdominal cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and adjusting the pressure the device exerts onto said aneurysm.

An alternative method of treating an aneurysm of a mammal patient by providing the medical device including any feature disclosed herein, comprising the steps of:
inserting a needle or a tube like instrument into the patient's thoraxial cavity,
using the needle or tube like instrument to fill a part of the patient's body with gas and thereby expanding said thoraxial cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and
adjusting the pressure said device exerts onto said aneurysm.

An alternative method of treating an aneurysm of a mammal patient by providing the medical device including any feature disclosed herein, said method comprising the steps of:
cutting the skin in the abdominal or thoraxial wall of said mammal patient,
dissecting an area of the aneurysm,
placing said device onto said aneurysm, and
starting the stimulation device and adapted to adjust any parameter related to said stimulation.
adjusting the pressure said device exerts onto said aneurysm.
adjusting the pressure said device exerts onto said aneurysm.

A computer program product comprising computer program segments that when executed on a computer causes the computer to control the pressure applied by an implantable member adapted to be placed in connection with a blood vessel having an aneurysm.

A digital storage medium comprising the computer program product.

### Fourth Combination Embodiments Includes a Monitoring/Sensor System Putting Pressure on the Aneurysm Including any Feature in any Combination, Comprising

A device for monitoring an aneurysm of a human or mammal patient comprising:

A sensor placed in relation to a wall portion of the aneurysm for generating a signal corresponding to a parameter related to the aneurysm or the treatment of the aneurism.

A device, wherein the parameter corresponds to the size of the aneurysm.

A device, wherein the parameter corresponds to the diameter of the aneurysm.

A device wherein the sensor is a gauge sensor.

A device wherein the parameter corresponds to a pressure.

A device wherein the pressure corresponds to a pressure from a hydraulic cuff provided around the aneurysm.

A device wherein the pressure corresponds to a pressure from a mechanical implantable member provided around the aneurysm.

A device wherein the pressure corresponds to a pressure in a blood vessel.

A device wherein the sensor is adapted to measure the pressure exerted on an implantable member provided around the aneurysm.

A device wherein the sensor is adapted to measure the volume of a hydraulic implantable member.

A method of treating an aneurysm of a mammal patient by providing the medical device, comprising the steps of:
inserting a needle or a tube like instrument into the patient's abdominal cavity,
using the needle or tube like instrument to fill a part of the patient's body with gas and thereby expanding said abdominal cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and
monitoring the expansion of the aneurysm by measuring the expansion the aneurysm exerts onto the device.

A method of treating an aneurysm of a mammal patient by providing the medical device, comprising the steps of:
inserting a needle or a tube like instrument into the patient's thoraxial cavity,
using the needle or tube like instrument to fill a part of the patient's body with gas and thereby expanding said thoraxial cavity,
placing at least two laparoscopic trocars in said cavity,
inserting a camera through one of the laparoscopic trocars into said cavity,
inserting at least one dissecting tool through one of said at least two laparoscopic trocars,
dissecting an area of an aneurysm of a blood vessel,
placing the device onto said the aneurysmic blood vessel, and
monitoring the expansion of the aneurysm by measuring the expansion the aneurysm exerts onto the device.

A method of treating an aneurysm of a mammal patient by providing the medical device, said method comprising the steps of:
cutting the skin in the abdominal or thoraxial wall of said mammal patient,
dissecting an area of the aneurysm,
placing said device onto said aneurysm, and
monitoring the expansion of the aneurysm by measuring the expansion the aneurysm exerts onto the device.

A method of treating an aneurysm of a mammal patient by providing the medical device, said method comprising the steps of:
cutting the skin of said mammal patient,
dissecting an area of the aneurysm,
placing said device onto said aneurysm, and
monitoring the expansion of the aneurysm by measuring the expansion the aneurysm exerts onto the device.

### K: Removing Blood Clots in the Vascular System

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for removing blood clots in the vascular system of a patient. Examples of such devices for removing blood clots in the vascular system of a patient will now be described.

An object is to provide a device, a system, and a method for removing blood clots in the vascular system of a patient.

The inventor has realized that a blood clot removal device can be implanted in a patient instead of being external of the patient.

According to a first aspect of the present invention there is provided a blood clot removal device for removing blood clots from the vascular system of a patient, the blood clot removal device being implantable in the patient's body and comprising a blood flow passageway to be connected to the patient's vascular system to allow circulation of the patient's blood through the blood flow passageway, a filter provided in the blood flow passageway for collecting blood clots occurring in the blood flowing through the blood flow passageway, and a cleaning device for moving blood clots collected by the filter out of the blood flow passageway.

In a preferred embodiment, the blood clot removal device comprises at least one fresh replacement filter, wherein the cleaning device is adapted to replace the filter, which is in the blood flow passageway together with collected blood clots, with the fresh filter. An operation device may be provided to operate the cleaning device to exchange the filter.

The operation device may be adapted to operate the cleaning device to clean the exchanged filter, for being ready for later reinstallation in the blood flow passageway. The cleaning device may be adapted to slice, push or scratch away any particles from the filter. Alternatively, the cleaning device may be adapted to clean the filter by sucking away any particles from the filter

The filter may be adapted to move blood clots to a location within the body of the patient, and may be adapted to take into account the fibrotic capsula covering the cleaning device when implanted.

The cleaning device may comprise a rotating cassette adapted to rotate the filter out from the blood flow passageway. The rotating cassette may move the filter out from the blood flow passageway together with particles collected by the filter and move a fresh replacement filter to the blood flow passageway. The cassette may contain one or two or more replacement filters, for example three replacement filters, wherein the cassette is adapted to revolve, for example by means of a motor, to replace the filter in the fluid passageway with one of the additional replacement filters. The cleaning device may be adapted to allow the replacement filters to be inserted into the cassette while replacing dirty filters therein. A motor may be provided for actively inserting one of the replacement filters into said cassette, to replace a dirty filter therein.

The blood clot removal device may comprise a reservoir for fresh and dirty filters.

The cleaning device preferably is adapted to move blood clots away from the blood flow passageway to a place free inside the patient's body, where the patient's body itself will take care of the blood clots. Alternatively, a collecting volume, such as a bag, may be provided for collecting blood clots that has been mechanically cleaned from the filter. Most likely such a bag will then be placed inside the body.

With the filter remaining in the blood flow passageway, the cleaning device may be adapted to slice, push or scratch away any clots from the filter, or to suck away any clots from the filter.

In one embodiment, the cleaning device comprises a first piston, with preferably is provided with a first recess in an outer end portion thereof to collect blood clots removed from the filter. By providing the first piston with a plurality of channels for accommodating the filter in an extended position of the first piston, it can surround the filter, ensuring essentially complete removal of blood clots therefrom. This is preferably effected if the first piston is movable in a direction perpendicular to the direction of the blood flow passageway.

The movement of the first piston can be controlled by a source of pressurized air, ensuring rapid acceleration of the first piston and thereby short cleaning cycles. The movement of the first piston can alternatively be controlled by an electric motor, a solenoid or the like.

The filter is of biocompatible material in order to avoid unnecessary interference with the blood environment.

In one embodiment, the blood clot removal device is used in a system comprising a heart pump connected to the blood flow passageway, wherein a first tube preferably connects the heart pump to the left ventricle of the patient's heart and a second tube connects the heart pump connected to the patient's aorta.

The filter preferably comprises a plurality of strips, which may be equally spaced to form a filter for blood clots. In order to achieve a filtering function, the distance between two adjacent strips is preferably less than 2 millimeters, and even more preferably less than 1.0 millimeter. The distance depends on which size blood clots you want to avoid.

In one embodiment, a second piston is provided across the blood flow passageway from the first piston, wherein the second piston is movable in a direction essentially perpendicular to the direction of the blood flow passageway and spring biased in the direction of the first piston. If an outer end portion of the second piston is provided with a second recess, the first piston and the second piston cooperate to catch blood clots for further removal. This further removal can be accomplished by means of a third piston, which is movable in a direction perpendicular to both the direction of the blood flow passageway and the direction of movement of the first piston and of the second piston.

In a preferred embodiment, the blood flow passageway of the blood clot removal device has an essentially square cross-sectional shape, which provides for a laminated flow of the blood, particularly if the square shape is combined with a filter comprising parallel strips.

The blood clot removal device is in a preferred embodiment insertable in a blood vessel of the patient, preferably via surgery.

The blood clot removal device can be adapted to be placed in the patient's abdomen or thorax.

The blood clot removal device is preferably comprised in a system for removing blood clots of a patient. This system can comprise a switch, preferably a subcutaneous switch being adapted to manually and non-invasively control any function of the blood clot removal device.

The system for removing blood clots preferably comprises a hydraulic device having a hydraulic reservoir, wherein the blood clot removal device is adapted to non-invasively be regulated by manually pressing the hydraulic reservoir.

A wireless remote control can non-invasively regulate any function of the blood clot removal device. Even more important any function of the device may be programmable by such a remote control.

Also, a wireless energy transmitter can non-invasively energize the blood clot removal device.

An energy source is preferably adapted to power the blood clot removal device. The energy source can comprise an internal energy source, such as an internal energy source adapted to receive energy from an external energy source transmitting energy in a wireless mode. The internal energy source can then be charged from the energy in the wireless mode.

The system preferably comprises a feedback device for sending information from inside the patient's body to the outside thereof to give feedback information related to at least one functional parameter of the device or a physical parameter of the patient, thereby optimizing the performance of the system. One preferred functional parameter of the device is correlated to the transfer of energy for charging the internal energy source.

The system preferably comprises an operation device for operating the blood clot removal device. This operation device can comprise a motor or a pump, an electrically powered operation device, a hydraulic operation device, or an electric motor.

To improve the performance of the system for removing blood clots, a physical parameter sensor, such as a pressure sensor, is provided for sensing a physical parameter of the patient. An internal control unit can act in response to the physical parameter sensed by the sensor.

A functional parameter sensor sensing a functional parameter of the blood clot removal device can also be provided. An internal control unit acting in response to the functional parameter sensed by the sensor can also be provided.

A method of using the system for removing blood clots is also provided, wherein at least one function of the blood clot removal device is regulated from outside the patient's body. The regulation is in a preferred embodiment non-invasively by manually pressing a subcutaneous switch. In an alternative embodiment, non-invasively regulation are performed by manually pressing a hydraulic reservoir connected to the blood clot removal device.

Alternatively, the system for removing blood clots comprises a wireless remote control, wherein non-invasively regulation is performed using said remote control.

In a preferred embodiment, the system for removing blood clots comprises a wireless energy transmitter, wherein non-invasively regulation is performed using said energy transmitter.

Preferably, an energy source is used for powering and adjusting any function of the blood clot removal device. The energy source may comprise an internal energy source, which preferably is associated with an external energy source adapted to transmit wireless energy. Energy is preferably transmitted from the external energy source to charge the internal energy source. Feedback information is preferably sent from inside the body to the outside thereof to give feedback related to the functional parameters of the device or physical parameters of the patient. The functional parameter of the device is correlated to the transfer of energy for charging the internal energy source.

In one embodiment, wireless energy is transmitted for powering the operation device.

In a preferred embodiment, the method of using a system for removing blood clots comprises the steps of: implanting an implantable source of energy in the patient, providing an external source of energy, controlling the external source of energy to release wireless energy, charging non-invasively the implantable source of energy with the wireless energy, controlling the implantable source of energy from outside the patient's body, and releasing energy for use in connection with operation of the blood clot removal device. The wireless energy is preferably stored in the implantable source of energy.

In another preferred embodiment, the method of using a system for removing blood clots comprises the steps of: providing an external source of energy outside the patient's body, and controlling the external source of energy from outside the patient's body to release wireless energy, and using released wireless energy for operating the operation device. The wireless energy is preferably transformed into electrical energy inside the patient's body using an implanted energy-transforming device and using the electrical energy when operating the blood clot removal device.

In one embodiment, the electrical energy is used directly in connection with operation of the blood clot removal device, as a transforming device transforms the wireless energy into the electrical energy.

In another embodiment, the external source of energy is controlling from outside the patient's body to release non-magnetic wireless energy, and released non-magnetic wireless energy is used for operating the blood clot removal device.

In yet an alternative embodiment, the external source of energy is controlled from outside the patient's body to release electromagnetic wireless energy, and released electromagnetic wireless energy is used for operating the blood clot removal device.

A method for placing a blood clot removal device, comprising a surgical method via a laparoscopic abdominal approach, comprises the steps of: inserting a needle or tube like instrument into the abdomen of the patient's body, using the needle or tube like instrument to fill the patient's abdomen with gas thereby expanding the patient's abdominal cavity, placing at least two laparoscopic trocars in the patient's body, inserting a camera through one of the trocars into the patient's abdomen, inserting at least one dissecting tool through a trocar and dissecting the intended placement area of the patient, placing at least one blood clot removal device in any part of the vascular system in the abdomen, moving blood clots away from the vascular system post operatively to a different part of the body, and powering the device with a source of energy. The blood clots can be moved either to a place that is free in the abdomen, to a place that is free in the abdomen, or to a place that is encapsulated in a closed bag in the abdomen

The blood clot removal device is preferably programmable from outside the patient's body. By adding the steps of sensing a physical parameter of the patient or a functional parameter of the device, and sending sensing information to an internal control unit adapted for regulating said blood clot removal device, performance is improved.

A method of placing a blood clot removal device, comprising a surgical method via a laparoscopic thoraxial approach, comprises the steps of: inserting a needle or tube like instrument into the thorax of the patient's body, using the needle or tube like instrument to fill the thorax with gas thereby expanding the thoraxical cavity, placing at least two laparoscopic trocars in the patient's body, inserting a camera through one of the trocars into the thorax, inserting at least one dissecting tool through a trocar and dissecting the intended area of the patient, placing at least one blood clot removal device in any part of the vascular system in the thorax, moving blood clots away from vascular system post operatively to a different part of the body, and powering the device with a source of energy. The blood clots can be moved either to a place that is free in the thorax, to a place that is free in the abdomen, or to a place that is encapsulated in a closed bag in the thorax

The device is preferably programmed from outside the body of the patient.

A method for surgically treating a patient needing a blood clot removal device in the vascular system in the patient's abdomen comprises the steps of: cutting an opening in the patient's abdominal wall, dissecting an area of the vascular system, placing a blood clot removal device inside the vascular system, and suturing said abdominal wall. In one embodiment, blood clots are moved away from the vascular system into an encapsulated closed bag in the patient's abdomen by means of the blood clot removal device. In another embodiment, blood clots are moved to the free abdomen.

A method for surgically treating a patient needing a blood clot removal device in the vascular system in the thorax comprises the steps of: cutting an opening in the thorax wall, dissecting the area of the vascular system, placing a blood clot removal device inside the vascular system, and suturing said thorax wall. A step of moving blood clots away from the vascular system can comprise moving blood clots either to a place that is free in the thorax, to a place that is free in the abdomen, or to a place that is encapsulated in a closed bag in the thorax

A method using an system for removing blood clots, for postoperatively and non-invasively regulating the blood clot removal device, comprises the steps of moving any blood clots, which have been accumulated in the vascular system of the patient's body, away from the vascular system, and placing the blood clots outside the vascular system. This can be accomplished by an energy source, preferably repeatedly according to a pre-programmed time-schedule. The steps of moving any blood clots away from the vascular system and placing the blood clots outside the vascular system are preferably repeated and at least partly controlled by an internal control unit getting input from a sensor sensing any physical parameter of the patient or any functional parameter of the device. The accumulation of blood clots in the filter may be visualized with light sensors, sensors measuring any electrical parameter, any blood flow measurement, pressure difference before and after the filter or any other kind of sensor.

An operation method for surgically placing a blood clot removal device comprises the steps of cutting the patient's skin, dissecting a placement area where to place the blood clot removal device inside the vascular system in the abdomen or thorax or retroperitoneal or subcutaneously or any limb of the patient, placing the blood clot removal device in the placement area, removing, postoperatively and non-invasively without penetrating the patent's skin, any blood clots from the vascular system to outside thereof, and using energy from an energy source without any penetration through the patient's skin to power the blood clot removal device.

### L: Treating Urinary Retention of a Mammal

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for treating urinary retention of a mammal patient, examples of such devices for treating urinary retention of a mammal patient will now be described.

In general terms, the present invention relates to an apparatus for treating urinary retention of a mammal patient, comprising an implantable powered member adapted exert a force from the outside on a selected part of the urinary bladder in order to discharge urine from the urinary bladder. The apparatus further comprises a control device for controlling the operation of the powered member. The force of the powered member is exerted at least partly against a support structure which is adapted to support against at least one of, a bone, such as the pelvic bone, pubic bone or sacrum or spinal cord, other human tissue such as peritoneum, the abdominal or pelvic wall or the urine bladder itself.

The control device preferably comprises a source of energy for operating the powered member and other energy consuming parts of the apparatus. Arrangements for energizing and controlling the apparatus in the context of a system comprising the apparatus will be disclosed below. The control device preferably is adapted to be implanted at least partly subcutaneously or in the abdomen or in the pelvic region. The control device comprises a control assembly adapted to be implanted both subcutaneously and/or in the abdominal cavity, said control assembly comprising at least two parts adapted to be connected, when implanted.

In order to actuate the urinary bladder from the outside, the powered member comprises a contacting part adapted to contact a surface part of the urinary bladder. The powered member comprises at least one operable pressurizer connected to the contacting part in an arrangement, wherein operating the pressurizer provides compression or release of the urinary bladder. For this purpose, the powered member can be hydraulically or mechanically operated to provide compression or release of the urinary bladder.

In one embodiment, the pressurizer comprises at least one movable arm extending from an operation device to the contacting part of the powered member. The operation device is adapted to displace the movable arm towards the urinary bladder in order to discharge urine from the urinary bladder. The operation device is fixated to human tissue, preferably in this embodiment, to the pubic bone. Further in this embodiment, the operation device comprises a motor, preferably an electric motor adapted to displace the movable arm. The contacting part is adapted be fixated to the upper part of urinary bladder and the contacting part preferably is designed to extend radially from a point essentially in line with the urinary bladder apex.

In another embodiment, the pressurizer comprises a reservoir for hydraulic fluid, and the contacting part comprises an expandable cavity hydraulically connected to the reservoir. The pressurizer comprises a pump for transporting the hydraulic fluid from the reservoir to expand the expandable cavity thereby compressing the urinary bladder. Further, the pressurizer is adapted to have the hydraulic fluid transported from the expandable cavity to the reservoir by the urinary pressure in the urinary bladder, when the pump is not active. In order to accomplish transportation back from the cavity to the reservoir, an arrangement can be provided wherein a second connection between the expandable cavity and the reservoir adapted to admit transportation hydraulic fluid from the expandable cavity to the reservoir by the urinary pressure in the urinary bladder, when the pump is not active. Preferably, the flow capacity of the second connection is smaller than the pump flow, allowing said second connection to stand open. Alternatively to this arrangement, the pump can transport hydraulic fluid from the expandable cavity to the reservoir in order to release the urinary bladder.

In still another embodiment, the operable pressurizer comprises an operation device attached to a support device adapted to be fixated to the urinary bladder wall. The operable pressurizer comprises an actuator operably connected to the operation device comprising a motor to perform an actuating movement to actuate the contacting part to compress the urinary bladder. Preferably, the operation device comprises a pivot for accomplishing a pivotal movement of actuator. The support device is generally ring-shaped or having an intermittent ring-shape and extends along the periphery of the urinary bladder.

The apparatus as embodied in previous sections further can comprise a device for electrically stimulating the muscles of the urinary bladder to contract. Such a stimulating device can comprise a plurality of electrode strips attached to the muscles of the urinary bladder.

The apparatus as embodied in previous sections can also comprise an implantable pair of restriction devices, wherein the control device controls the restriction devices adapted to close the ureters when discharging urine from the urinary bladder.

The apparatus as embodied in previous sections can also comprise an artificial urinary sphincter, wherein a restriction device, controlled by the control device performs as a urinary sphincter.

The apparatus as embodied in previous sections can also comprise a sensor for measuring any parameter related to the urinary pressure or volume of the urinary bladder. The sensor is capable of sending a signal to the control device, which thereby activates and deactivates the powered member. In general terms the present invention relates to an apparatus for treating urinary retention of a patient by discharging urine from the urinary bladder, comprising an expandable member adapted to be implanted inside the urinary bladder of the patient, and an implantable control device for controlling the volume of the expandable member. The control device is adapted to be connected to the expandable member through the wall of the urinary bladder. As a result of the expansion of the expandable member urine is discharged from urinary bladder through the urethra. Further to this general embodiment, the control device comprises a powered operation device for assisting the expandable member to discharge urine from the urinary bladder, and the apparatus further comprises an external energy transmission device capable of wirelessly transmitting energy from the outside of the patient's body to the inside of the patient's body to be used in connection with operating the powered operation device and other energy consuming implantable parts of the apparatus.

It will be evident from the present invention to be described as follows that the term “control device” has a meaning including both hydraulic and electric components of the apparatus assisting with the urinary discharge from the bladder. These components include both implantable components and components intended to be outside the patient's body. In this context it should be observed that the control device could be deviated into a hydraulic control device and an electric control device. The electric control device may then include power supply and electrical control functions as well as a wireless energy receiver. The external control device could also be described as the external control unit for transmitting wireless energy and receiving feedback information from implanted components. Therefore in any place in this document describing the control device this term could be replaced by any of the forgoing, whenever relevant. In one embodiment the control device comprises an internal control unit comprising at least one of a subcutaneously placed switch, an electronic circuit, a motor or a pump, wherein said internal control unit is operable from the outside of the patient's body.

The expandable member preferably is releasably attached to the control device with a detachable coupling. For this purpose the expandable member, preferably is provided with a first mating part fitting with a second mating part of the control device. The mating parts can be a combination of male/female parts which together establish a releasable coupling that readily attaches or detaches the expandable member and the control device. Preferably, the mating parts together provide a snap lock coupling that simplifies the replacement of the expandable member through the urethra. Accordingly, the expandable member is designed with a capacity to assume an essentially cylindrical elongated shape which admits its transportation through urethra assisted with a suitable surgical instrument. The expandable member, as inserted for implantation can comprise a bellows or a similar structure undergoing controlled expansion and collapse. Preferably, the expandable member is hydraulically controlled and comprises a cavity for hydraulic fluid and the control device comprises a bladder operating reservoir for hydraulic fluid. The expandable member and the control device are accordingly adapted to be hydraulically connected through the wall of urinary bladder. For this purpose, the control device preferably comprises a tube to establish hydraulic connection and for transporting the hydraulic fluid between the bladder operating reservoir and the cavity. The quick coupling can in one embodiment be connectable to the hydraulic connection and its mating part establish a connection between the expandable member and the bladder operating reservoir so hydraulic fluid can be transported to and from the expandable member for discharging urine and when the urinary bladder is refilled,

The powered operation device transports hydraulic fluid to and from the cavity and the bladder operating reservoir. In one mode of operation, the expandable member is adapted to be emptied by the pressure exerted by urine of the urinary bladder to transport the hydraulic fluid from the cavity to the bladder operating reservoir. The operation device is capable of transporting hydraulic fluid to cavity of the expandable member to obtain a suitable urinary pressure for discharging urine. Preferably a urinary pressure of at least 50 cm water pressure for discharging urine is obtainable.

Preferably the operation device is a powered pump. Further, the operation device can comprise or being connected to an injection port, to calibrate the amount of hydraulic fluid. The operation device can also be manually operated by an injection port which is operated from outside the body by filling or emptying said injection port.

In addition, the apparatus can comprise implantable restriction devices adapted to close the ureters when discharging urine from the urinary bladder in order to prevent any urinary backflow towards the kidneys. The restriction devices preferably are adapted to open and close the ureters are hydraulically operable by hydraulic fluid. In a suitable embodiment the operating hydraulic fluid is displaced from the bladder operating reservoir. For this purpose, the bladder operating reservoir can comprise a sealed expandable/collapsible section for the restriction device operating hydraulic fluid. Preferably, these restriction devices open and close by the activity of the operation device.

The apparatus can also comprise a restriction device adapted to open and close the urethra to assist patients having an impaired urinary sphincter function. Restriction devices suitable for the urinary tract and wireless control of such devices are further described in European Patents Nos. EP 1253880; EP and EP 1263355, incorporated here as references.

### M: Breast Implant System

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a breast implant system. Examples of such breast implant systems will now be described.

It is therefore a primary object of the present invention to provide breast implants or, more specifically, a breast implant system, that offers new possibilities for varying the shape of the breast implant after implantation.

A breast implant system according to the present invention comprises a plurality of chambers, including one or more first fluid chambers and one or more second fluid chambers. At least the first fluid chambers are to be implanted in the human body to form part of a breast implant. The second fluid chambers are either implanted also to form part of the breast implant, or are implanted inside the patient's body remote from the breast implant. The first fluid chambers are interconnected with the second fluid chambers, such that fluid can be exchanged between the first and second fluid chambers, so as to change their respective fluid content. In specific embodiments, further (third) chambers can be present, which may or may not be in fluid connection with the first or/and second fluid chambers and which need not necessarily contain any fluid but may even contain non-flowing material.

A fluid in the sense of the present invention encompasses any flowable material that can be exchanged between the first and second fluid chambers, in particular a gas, a liquid, a gel, a flowable foam or any combination thereof.

The exchange of fluid between the first and second implanted fluid chambers, resulting in a change of the respective fluid content in the fluid chambers, allows the shape of the breast implant to be varied without the need to administer or remove any fluid through the patient's skin. This is very convenient for the patient, as there is no valve sticking out through the skin and there is no need to perform minor surgery for accessing any valve located underneath the skin. This does not preclude there also being provided in the present invention an injection port arranged subcutaneously in order to refill the content of any of the chambers or to release any excess fluid, should need arise.

It is thus possible to implant the breast implant system of the present invention completely in the patient's body (apart from any external wireless remote-control elements, if present, as will be described below).

As mentioned above, one or more of the second fluid chambers or even all chambers of the breast implant system may form part of the breast implant. Thus, according to a preferred embodiment, the shape of the breast implant can be changed without changing the overall volume of the breast implant, namely in the case where a second fluid chamber forms part of the breast implant along with the first fluid chamber, and fluid is exchanged between this second fluid chamber and the first fluid chamber. For instance, the patient may simply compress one of the fluid chambers within the breast implant from outside the breast to urge fluid to flow into a neighboring fluid chamber within the breast implant, and vice versa, until achieving a proper fluid distribution among the various fluid chambers so as to give the breast implant the personally most preferred shape.

One or more third fluid chambers may further be provided in the breast implant to participate when fluid is exchanged between the first and second fluid chamber. For instance, the third fluid chamber may be interposed between the first and second fluid chambers and may comprise means, such as a pump, to support the fluid exchange between the first and second fluid chambers.

The first and second fluid chambers may be separated from one another by a non-stretchable wall, which can be flexible or rigid, but have stretchable exterior walls. The non-stretchable separating wall guarantees that upon compression of the chambers, fluid will flow from the compressed fluid chamber through the non-stretchable wall into an adjacent chamber with a stretchable exterior wall. Provided that the flow path between the two adjacent fluid chambers includes a valve which opens in the one and/or the other direction only when a predetermined pressure difference is exceeded, the non-stretchable separating wall hinders the high pressure in one chamber from being transmitted by the separating wall to the fluid in the adjacent chamber. Thus, it is possible to have different pressures in different fluid chambers. This way, the number of shape variations of the breast implant is increased.

According to other embodiments of the invention, not only the shape of the breast implant is varied but also the volume thereof. This can be achieved by adding fluid to or removing fluid from the first fluid chambers in the breast implant from outside the breast implant. In this case the mass of the breast implant changes. However, according to a specific embodiment of the present invention, the shape and also the volume of the breast implant are changed with the mass of the breast implant remaining constant. This can be achieved with different types of fluid in the fluid chambers, i.e. at least one fluid chamber comprising a compressible fluid and at least one other fluid chamber comprising an incompressible fluid. More specifically, the first fluid chamber within the breast implant may comprise an incompressible fluid and the second fluid chamber within the breast implant a compressible fluid, and a transfer of the incompressible fluid from the first fluid chamber to the second fluid chamber will result in an increase of pressure within the second fluid chamber and, thus, in a decrease of volume of the compressible fluid contained in the second fluid chamber. Again, this effect will be enhanced when a non-stretchable wall, which may be flexible or rigid, is arranged between the first and second chambers. More preferably, the non-stretchable wall may completely enclose the fluid chamber comprising the compressible fluid. For instance, the fluid chamber comprising the compressible fluid may be placed within the fluid chamber comprising the incompressible fluid.

When an incompressible fluid is transferred into the chamber comprising a compressible fluid, it may be difficult to ensure that upon the next shape variation of the breast implant only the incompressible fluid and no compressible fluid is moved back into the chamber with incompressible fluid. It is therefore preferred to provide the compressible fluid in an isolated chamber. In this case, both the first and second fluid chambers in the breast implant comprise an incompressible fluid and the exchange of the incompressible fluid between the first and second fluid chambers results in an increase of pressure within the third fluid chamber that also forms part of the breast implant and that is filled with the compressible fluid. Thus, the volume of the third fluid chamber will decrease accordingly, and so will the overall volume of the breast implant.

This way, the shape of the breast implant can be changed from flat to high and vice versa, this involving a volume change of the breast implant, while the weight of the breast implant is not affected.

Where at least one of the second fluid chambers is implanted remote from the breast implant in the patient's body, and where the shape of the breast implant is changed by exchanging fluid between the first fluid chamber or chambers in the breast implant and the remotely implanted second fluid chamber, such shape variation will involve a change of the breast implant's volume (and weight). Thus, the size of the breast implant will increase accordingly.

There may be one or more first fluid chambers provided in the breast implant and/or there may be one or more second fluid chambers remotely implanted. Each of the remotely implanted second fluid chambers can be connected to one or more of the first fluid chambers in the breast implant. Also, each of the first fluid chambers in the breast implant may be connected to one or more of the remotely implanted second fluid chambers. Depending on how the first and second fluid chambers are interconnected, a great variety of shape variations can be achieved with the breast implant system. One or more valves can be provided to control fluid flow between the fluid chambers.

Again, a third chamber isolated from the first and second fluid chamber can be provided and may have different functions. For instance, the third chamber may form part of the breast implant and may contain a compressible fluid, the arrangement being such that fluid exchange between the first and second fluid chambers containing an incompressible fluid results in a change of pressure within the at least one third chamber containing the compressible fluid, thereby causing a change of volume of the breast implant.

In another embodiment of the invention, the third chamber is a fluid chamber cooperating with the first and second fluid chambers such that when fluid is exchanged between the first and second fluid chambers the fluid in the third fluid chamber is caused to move, the amount of fluid moved in the third fluid chamber being different to the amount of fluid exchange between the first and second fluid chambers. This can also be referred to as a servo system.

According to one preferred embodiment of such servo system, the third fluid chamber comprises sub-chambers which are interconnected. At least one of the first and second fluid chambers is operatively connected to at least one of the sub-chambers and is adapted to expand this sub-chamber when fluid is exchanged between the first and second fluid chambers. Fluid will then flow between the sub-chambers into the expanded sub-chamber, and the amount of fluid flow can be substantially different to the amount of fluid exchange between the first and second fluid chambers. There are various alternative ways of realizing such servo system in the breast implant system of the present invention. In this context, it is preferable to provide a spring element to urge the third fluid chamber or at least one of the sub-chambers thereof into a state of minimum or maximum volume, i.e. into a normally small or a normally large state. Energy is then only needed to exchange fluid between the first and second fluid chambers in one direction, whereas the necessary force required to exchange the fluid in the opposite direction is provided by the spring force, which spring force can be released up to an appropriate amount according to the patient's preferences.

The servo system can be designed as a reverse servo system to the extent that only a little amount of fluid needs to be exchanged between the sub-chambers of the third fluid chamber in order to achieve a relatively large amount of fluid exchange between the first and second chambers. This means that a relatively large force but small stroke is needed to achieve the relatively large amount of fluid exchange between the first and second fluid chambers. This is particularly convenient where one of the sub-chambers of the third fluid chamber is provided for subcutaneous implantation so as to be manually compressible by the patient from the outside of the patient's body. Thus, the subcutaneously arranged compressible sub-chamber may have a relatively small volume and will therefore not adversely affect the patient's visual appearance, with the negative side effect that the patient will have to apply a relatively large force on the relatively small subcutaneous sub-chamber in order to achieve the desired, relatively large volume change in the breast implant.

In cases where the breast implant system does not include a servo system or, thus, any sub-chambers of any third fluid chamber, the remotely implantable second sub-chamber may itself be arranged for subcutaneous implantation so as to be operable by the patient from outside the patient's body.

Instead of any fluid chamber being arranged under the patient's skin for manual operation by the patient, it may be implanted deeper in the patient's body, such as in the patient's abdominal cavity or inside the patient's chest area, where it is preferably implanted outside the patient's thorax, in particular under the pectoralis muscle. In this context the breast implant system would comprise a pump for pumping the fluid between the fluid chambers inside the breast implant and those fluid chambers remote from the breast implant. Of course, a pump can also be useful in the case where fluid is exchanged between two or more fluid chambers inside the breast implant.

When implanting one or more of the second fluid chambers of the system under the pectoralis muscle, it may be implanted either underneath the patient's minor pectoralis muscle next to the patient's thorax or between the patient's minor and major pectoralis muscles. In either case, the second fluid chamber is preferably wide and flat as compared to the first fluid chamber of the breast implant, i.e. it has a substantially larger surface-to-volume ratio than the first fluid chamber. This way, a substantial subjective volume change of the patient's breast can be achieved by exchanging fluid between the first and second fluid chambers. The arrangement of the second fluid chamber between the major and minor pectoralis muscles can be more convenient for the patient as compared to the arrangement next to the patient's thorax.

According to a preferred aspect of the present invention, the breast implant is made to be light weight. Therefore, at least one of the plurality of chambers is at least partly filled with a gas or with another light-weight material that has a density substantially lower than the density of water. The chamber filled with the light weight material need not necessarily be in fluid connection with the first and second fluid chambers. It may be isolated from the first and second fluid chambers, as in the previously mentioned embodiment with a fluid chamber comprising a compressible fluid.

Instead of a gas, a non-flowable foam may be provided, and the foam may be filled with gas. The non-flowable foam may likewise be filled with a material resembling human material, such as a collagen. Also, a part of the non-flowable foam may form closed cells which are preferably filled with gas or with another light weight material, such as collagen. Another part of the non-flowable foam may be an open cell foam which may be arranged to absorb at least part of the fluid to be exchanged between the first and second fluid chambers.

The chamber with the light weight material may also include a soft material, such as silicone or any other gel-like material, with bubbles dispersed therein. Again, the bubbles may be filled with a gas and/or material that resembles human material, such as collagen.

According to another aspect of the breast implant system, in order to provide stiffness giving the breast implant a basic contour which is maintained throughout any shape changes of the breast implant, a rigid back wall may be provided to be placed adjacent the patient's thorax. At least the first chamber forming part of the breast implant is fixedly connected to the rigid back wall. The first fluid chamber and the rigid back wall may together conveniently form an enclosed space.

Problems can arise with fibrosis forming on the outer surface of the breast implant. Such fibrosis becomes relatively strong and may obstruct volume changes of the breast implant. It is therefore preferable to design the breast implant such that the outer walls thereof do not change their outer surface area upon an increase or decrease of the breast implant's volume. For instance, the breast implant may have a flexible outer wall, which is preferably non-stretchable, and the flexible outer wall may be formed like a bowl, so that the shape of the bowl can be changed without changing the outer surface area thereof. Again, the flexible outer wall may be mounted on a stiff frame to guarantee a basic contour of the breast implant.

In a specific embodiment, the flexible outer wall is provided with one or more folds or pleats so as to allow a flexible movement of the outer wall by the folds or pleats being unfolded upon an increase of an inner volume of the breast implant. Since fibrosis tends to build up in the inner corners of a zig-zag-shaped wall, it is preferred that the folds—in cross section—comprise trapezoidal sections.

However, it is not excluded that the flexible outer wall is at least partly stretchable so as to allow, where possible, stretching of the outer wall upon an increase of an inner volume of the breast implant.

In order to improve the overall appearance of the breast implant, the part of the outer wall facing away from the patient's chest may comprise a compartment filled with a soft material, such as a foam or a silicone. This gives the breast implant the look and feel of a natural breast and can also serve to level out any unevenness caused by different fillings and/or different pressures in the breast implant's fluid chambers.

After a while it may become necessary to remove fluid from one or more of the fluid chambers or, more probably, to add fluid thereto. In particular when gas is contained in one or more of the fluid chambers, it is possible that part of the gas will get lost over time due to leakage. Therefore, in order to maintain a desired balance in the fluid chambers, the breast implant system according to a preferred embodiment includes at least one injection port for implantation under the patient's skin so as to allow fluid to be added to or removed from the fluid chambers by injection from outside the patient's body. Two or more injection ports may be provided and may be connected to the fluid chambers via individual fluid connections so as to allow fluid to be individually added to or removed from specific fluid chambers. The injection port or ports can further be used to adjust the pressure in the breast implant system. For instance, when a patient has achieved a particular distribution of fluid among the first and second fluid chambers that feels best, it is convenient for the patient to release any excess pressure from the system by selectively removing fluid from the system through the injection port or ports.

Alternatively, the pressure in the fluid chambers may be equalized using a pressure relief valve. Such pressure relief valve may be provided at least for controlling the pressure in one or more of the first fluid chambers forming part of the breast implant.

As mentioned above, a pump may be provided in the breast implant system for pumping fluid between the first and second fluid chambers. In this context, at least one reservoir—i.e. a third fluid chamber—may be provided remote from the breast implant and connected to the pump so as to allow fluid to be exchanged at least between the first and second fluid chambers by pumping fluid with said pump from the first fluid chamber into the reservoir and from the reservoir into the second fluid chamber, and vice versa. Alternatively, the pump may be arranged between the first fluid chambers in the breast implant and a remotely implanted second fluid chamber. Instead of the pump being provided outside the first and second fluid chambers, it may be contained within one of the first and second fluid chambers.

As mentioned above, the pump may also be implanted subcutaneously so as to be manually operable through the skin. In this case, a purely hydraulic or purely pneumatic pump may be used.

However, where the pump is not manually operable, the breast implant system may comprise at least one motor for automatically driving the pump. In this case, the pump may be of the hydraulic, pneumatic or mechanical type. Also, a manually operable switch for activating the motor may be arranged subcutaneously for operation by the patient from outside the patient's body.

The motor itself may be arranged to be driven by electric or electromagnetic energy, by an electric or magnetic pulsating field or by ultrasonic energy.

The breast implant system may further comprise an energy source for supplying the energy directly or indirectly to at least one energy consuming part of the system, in particular to the aforementioned motor for driving the pump. Such energy source may include energy storage means, such as a battery or an accumulator, in particular one or more of a rechargeable battery and a capacitor.

The energy source, when provided outside the patient's body, preferably comprises a wireless energy transmitter for wirelessly transmitting energy from outside the patient's body to the implanted energy storage means.

The breast implant system preferably further comprises an implantable energy transforming device for transforming wirelessly transmitted energy into electric energy. The electric energy is stored in the energy storage means and/or is used to drive the energy consuming part (motor and pump) as the energy transforming device transforms the wireless energy into the electric energy. Alternatively, the energy consuming part may be adapted to directly transform the wirelessly transmitted energy into kinetic energy.

It is further preferable to provide a feedback subsystem adapted to wirelessly send feedback information relating to the energy to be stored in the aforementioned energy storage means from inside the human body to the outside thereof. The feedback information is used by the breast implant system to adjust the amount of wireless energy transmitted by the energy transmitter from outside the patient's body. The feedback information provided by the feedback system may be related to an energy balance which is defined as the balance between an amount of wireless energy received inside the human body and an amount of energy consumed by the at least one energy consuming part. Alternatively, the feedback information may relate to an energy balance which is defined as the balance between a rate of wireless energy received inside the human body and a rate of energy consumed by the at least one energy consuming part.

It is further preferred to provide the breast implant system with a control unit to directly or indirectly control one or more elements of the breast implant system. For instance, the control unit may be primarily adapted to control the exchange of fluid at least between the first and second fluid chambers. Preferably, such controlling action is carried out non-invasively from outside the patient's body, such as by wireless remote control. In this case, a part of the control unit is implanted in the patient's body whereas another part is not implanted. In particular in the case where the control unit is completely implanted in the patient's body, a manually operable switch for activating the control unit may be arranged subcutaneously so as to be operable from outside the patient's body.

Where one part of the control unit is provided outside the patient's body and the other part is implanted in the patient's body, the external part of the control unit may be used to program the implanted part of the control unit, preferably wirelessly. Also, the implantable part of the control unit may be adapted to transmit a feedback signal to the external part of the control unit.

The breast implant system of the present invention may be implanted in the patient's body by open surgery or laparoscopic surgery. Open surgery would include the following implantation steps:
cutting an opening in the skin in the breast area,
dissecting the area,
placing at least a part of the adjustable breast implant system in the dissected area, and
suturing the skin.

A laparoscopic method of implantation would include the following steps:
inserting a needle like tube into the breast area of the patient's body,
using the needle like tube to fill the breast area with gas thereby expanding a cavity,
advancing at least two laparoscopic trocars in the patient's body,
inserting a camera through one of the trocars,
inserting at least one dissecting tool through another one of the trocars and dissecting an area of at least one portion of the patient's breast area, and
placing at least a part of the adjustable breast implant system in the dissected area.

As mentioned in the outset, no part of the breast implant system should penetrate the skin when the skin is sutured.

It is an object of the present invention to provide a breast implant, or, more specifically, a breast implant system that allows for an easy manipulation of the breast implant when varying the shape thereof.

A breast implant system according to the present invention comprises at least one first element and at least one second element to be implanted in the patient's body to form part of a breast implant. The first element and the second element (or elements) each comprise an outer wall defining a volume which is filled or fillable with a filling material, i.e. they are provided as filled hollow bodies. Alternatively, either or both of the first and second elements may be provided in granular form. The first element is displaceable relative to the second element. That is, the first element can be moved, when implanted in the patient's breast, between different spots within the breast to change the outer shape of the breast. Thus, by simple manipulation from outside the patient's breast, the two or more elements can be rearranged within the breast. For instance, the two elements may be pad-like or cushion-like. When one element is positioned at least partly below or beside the respective other element, the breast will have a relatively flat shape. By manually manipulating the breast from outside, the
two elements may be relocated within the patient's breast, so that they lie on top of each other in the most extreme situation, in which case the breast will assume a relatively steep shape.

While in a most simple configuration the two (or three or even more) elements are freely moveable relative to each other, it is advantageous to limit their movability within certain boundaries. Therefore, at least one of the elements may be fixedly mounted. For instance, it is described in WO 2007/004213 A2 how an implant may be fixed to the substantially horizontal rib bones by means of straps having anchoring ends provided with bone anchoring elements. By means of those straps, one or more of the elements can be mounted to remain substantially stationary, whereas one or more of the remaining elements may be freely displaceable relative to the fixedly mounted element or elements. This configuration contributes to the breast maintaining a certain basic shape that can be changed in a limited way by relocating the remaining element or elements.

Alternatively, or, preferably, in addition to the stationary mounting of one or more elements, it is advantageous to interconnect the first and second elements, or at least some of them, to limit their relative displaceability. This constructional aspect further contributes to the breast maintaining a basic shape that can be modified manually from outside the patient's breast within certain limits.

It is even more advantageous when the first and second elements are mounted to be movable only between predetermined spots. This will minimize the efforts that have to be taken during the reshaping step, and will simplify the overall procedure. For instance, it will be easy for the user to change the shape of the breast implant from one extreme to another extreme, e.g. from flat to high, because the extreme positions are defined by the limits of movement of the elements. For instance, the elements may be mounted to each other by straps or may be otherwise interconnected, such as gluing together a section of the flexible walls of the pad-like or cushion-like elements. Alternatively or in addition, they may be hinged to the bone structure, as mentioned above.

However, most preferably the elements are contained in a casing in order to isolate them from living tissue. Living tissue will soon overgrow the elements if not contained in a casing and, thus, hinder manipulation and relocation of the elements within the breast. Within such casing, at least a first one of the elements would be movable between different spots.

At least a second one of the elements may form part of a wall of the casing so that it assumes a relatively fixed position within the breast. Only the first element would have to be moved between different spots within the casing by manipulation from outside the patient's breast. Again, such freely movable element may be displaced, e.g., from a position below or beside the fixedly mounted element, to a position on top thereof, so as to reshape the breast from relatively flat to relatively high. The casing itself has a flexible and preferably also stretchable wall, i.e. an elastic wall, to follow any shape changes caused by an internal relocation of the elements.

The flexibility or elasticity of the casing's outer wall is also important in respect of fibrosis covering said outer wall, when the breast implant is implanted in the body. The shape of the casing must be sufficiently flexible to allow lengthening of the functional length thereof without interfering with the fibrosis. For instance, the casing's outer wall may have at least one wrinkle or cease, similar to that of a bellows, to allow lengthening of the outer wall without lengthening fibrosis covering the outer wall when implanted.

The second element may form part of the wall of the casing, or more preferably, part of a rigid back plate of the breast implant. It is particularly advantageous to fixedly mount the second element within the casing, when the first and second elements are somehow interconnected in a manner that limits their relative movement. By mounting the second element fixedly to the casing, predetermined shapes of the breast implant are easy to obtain, because only the first element needs to be displaced, and the displacement can only occur within a predetermined range, due to the first element being connected to the fixedly mounted second element.

According to another embodiment, either, or both, of the first and second elements are mounted within the casing to be movable only between predetermined spots. Thus, the respective element is not fixedly mounted within the casing, but there is a range of spots between which it can be moved. This allows a general structure to be maintained, while permitting certain changes to be made within such structure.

It is even possible to fixedly mount the second element to a wall of the casing, and to mount the first element to be movable within the casing only between predetermined spots. For instance, the first element may be somehow connected to the second element, or may be connected somehow to a wall of the casing, or both. With this arrangement, it is possible to provide a structure in which one or more first element (or elements) is movable from a spot located at least partly beside the second element (which can likewise be said to be a spot above or below the second element, depending upon the perspective), to a spot on top of the second element, so as to change the outer shape of the breast implant, from relatively flat to relatively high.

It should be clear from the above that there are many possibilities of providing and arranging the first and second elements. This includes in particular that two, three, or even more of at least one of the first and second elements may be present.

Alternatively, all of the elements may be moveable within the casing between different spots. In particular, where the elements are not pad-like or cushion-like, but are small and present in an amount of ten or more, hundred or more, or even thousands, it is not necessary to limit their displaceability within the casing.

The actual purpose is in each case to change the outer shape of the breast implant post-operatively, and, in particular, non-invasively.

As mentioned before, the breast implant preferably has a rigid back wall arranged to being placed adjacent the patient's thorax. The back wall provides stiffness and gives the breast implant a basic contour that is maintained throughout any shape changes of the breast implant. The rigid back wall advantageously forms part of the casing. Again, the casing, i.e. preferably the rigid back wall thereof, may be mounted to the patient's bone structure in the manner described before.

In addition to or independent from any rigid back wall, the casing may have a structure comprising at least one compartment filled with a soft material, such as a foam or silicone. This gives the breast implant the look and feel of a natural breast, and can also serve to level out any unevenness caused by different fillings, and/or different pressures, in the elements contained in the breast implant's casing, or caused by an uneven distribution of the elements within the casing.

According to a particularly preferred embodiment, there is further provided a reservoir that comprises a lubricating fluid. The reservoir is connected to the casing to allow the lubricating fluid to be supplied to, and removed from, the interior of the casing. Surface friction between an outer surface of an element contained in the casing, and an adjacent surface of a different element contained in the casing, and/or of the casing itself, and/or of a different component of the breast implant, such as the rigid back plate, can be reduced by supplying lubricating fluid from the reservoir into the casing.

Thus, when one wishes to change the shape of the breast implant, lubricating fluid is first supplied from the reservoir to the casing, so that it can flow between contacting surfaces, thereby reducing surface friction. Next, the shape of the breast implant is changed in any desired manner, e.g., by manually rearranging the elements contained in the casing from outside the breast implant. It should be noted that—although preferred—the invention is not limited to fully implanted breast implant systems, i.e., components thereof may be provided outside the patient's body, such as the lubricating fluid reservoir and the like. Once the shape of the breast implant has been changed by changing the position of one or more elements contained in the casing, the lubricating fluid can be removed from the casing, so that surface friction of the outer surface of the element or elements will increase. The increased surface friction offers the important advantage that the shape of the breast implant obtained by the manipulation of the element or elements within the casing will substantially be maintained over a long period of time. This is particularly important in embodiments where the element or elements are not fixedly connected to a supporting structure, but are, e.g., freely movable within the casing.

Generally, the lubricating fluid may be a liquid or a gas. Where the lubricating fluid is a gas, the casing can be set under gas pressure to inflate the casing, and thereby facilitate manipulation of the element or elements contained in the casing. However, because it is typically dangerous to handle gas within the human body, the lubricating fluid used in connection with the present invention is preferably a liquid or a gel, in particular an isotonic liquid or gel. The use of an isotonic liquid will not cause any harm to the patient in case of any leakage.

The breast implant system of the present invention is preferably a fully implantable system (except possibly for a wireless remote control or wireless energy transfer from outside the patient to the implanted system), with no need for medical practitioners to intervene, in particular without any need for surgery, when the shape of the breast implant is altered. As such, it is preferred to implant the lubricating fluid reservoir within the patient's body along with the breast implant. Because implantation of the breast implant requires surgery in the patient's chest area, the lubrication fluid reservoir may advantageously also be implanted in this area and, more specifically, outside the thorax just like the breast implant. In order not to negatively influence the outer shape of the breast implant, it is preferred that the reservoir is adapted for implantation below the minor pectoralis muscle, or between the major and the minor pectoralis muscles.

However, in order to prevent the lubricating fluid reservoir from having any influence on the outer shape of the breast implant, it may be advantageous to adapt the reservoir so that it can be implanted within the patient's body, remotely from the breast implant, in which case at least one conduit will have to be provided between the remotely implantable reservoir and the casing, in order to allow for fluid exchange between them.

Implanting the lubricating fluid reservoir remotely from the breast implant may also be advantageous for reasons other than the visual appearance of the breast implant.

For instance, the lubricating fluid reservoir may be adapted to be implanted in the armpit, or underneath the patient's arm, where it is hidden but can easily be accessed, such as by the patient manually compressing the reservoir, and, thus, urging lubricating fluid to flow into the casing.

Alternatively, the lubricating fluid reservoir may be adapted for implantation in the patient's abdominal cavity, because the abdominal cavity generally offers more space for implantation, which is particularly advantageous if further components such as a pump and/or a motor and the like are to be implanted along with the reservoir.

It is even possible to implant only a part of the reservoir remotely from the breast implant, while another part is implanted as part of the breast implant, or next to the breast implant. For instance, where a servo system is used to exchange fluid between the reservoir and the casing, as will be described in more detail below, a first part of the reservoir may be implanted, e.g., in the armpit, or underneath the patient's arm, or in the patient's abdominal cavity, in such a way that it is easily accessible by the patient, in particular subcutaneously, and a more voluminous part of the reservoir may be adapted for implantation somewhere else in the patient's body, e.g., next to the breast implant.

As mentioned before, subcutaneous implantation of at least a part of the reservoir can be made, such that manually compressing the subcutaneously implanted part causes fluid to flow from the reservoir into the casing. Advantageously, there is at least one valve provided between the reservoir and the casing, restricting flow in the one and/or in the other direction. For instance, a one-way or two-way valve may be provided to prevent lubricating fluid from flowing back to the reservoir while the size and/or shape of the reservoir is being manipulated. Once a desired size/shape has been achieved, the user may compress the breast implant using both hands, thereby increasing the internal pressure in the casing to a value at which the valve opens, and the lubricating fluid will then flow back into the reservoir. Because only a small amount of lubricating fluid is needed to achieve the friction reducing effect, this procedure will not substantially affect the size/shape of the breast implant. Preferably, the valve is a two-way valve, which opens towards the casing when the (manual) pressure imparted on the reservoir exceeds a predetermined pressure, and opens towards the reservoir when a pressure applied on the casing from outside the breast implant exceeds a predetermined pressure.

In a preferred embodiment, the casing includes hundreds of small elements, and these elements may be granular or spherical, in particular, balls. Preferably, these elements should adhere together in the absence of the lubricating fluid. For instance, the elements may be formed as balls not completely filled, so that they have a slack outer surface. The contacting surfaces of adjacent balls is, thus, relatively large, so that the surface friction resisting relative movement between adjacent balls is likewise relatively large.

In the absence of lubricating fluid, the mutual positions of the elements within the casing is unlikely to change substantially over time, whereas the mutual positions of the elements can be easily changed in the presence of the lubricating fluid, e.g., by manual manipulation from outside the breast implant.

It is even within the scope of the present invention that more than one casing is provided in the breast implant. In particular, one or more casings, into which lubricating fluid can be supplied, may be contained within a larger casing, into which the lubricating fluid can likewise be supplied. Thus, the internal casing or casings may constitute “elements” within the larger casing. This way, it is possible to manipulate the elements within the internal casings, which may contain hundreds of small elements as described above, and to manipulate the internal casings within the larger casing, e.g., by rearranging their mutual position within the larger casing.

According to another preferred embodiment of the invention, the element or elements within the casing may have the form of a cushion or pad, as briefly mentioned before, similar to those typically used for increasing the female breast for aesthetic purposes. Such cushions or pads are filled with a material, and have a flexible outer wall, the wall forming the outer surface of the elements. For the reasons already set out before, the flexible outer wall of the cushions or pads are preferably slack to increase the contacting surface area, and, thus, increase surface friction with other cushions or pads in the absence of the lubricating fluid.

The filling material of the pad or cushion may be granular but preferably is a soft material, in particular a fluid. As such, the filling material may comprise gas, liquid, gel, foam or any other flowable material, or a combination of the aforementioned materials. Most advantageously, the filling material is a silicone liquid or a silicone gel, as the consistency thereof comes closest to that of a natural breast.

So far it has been described how the shape of a breast can be changed by relocating, within the patient's breast, one or more of the elements of the breast implant system. The variability of changing the breast implant's shape can be further improved by inter-exchanging content between two or more elements. Of course, these elements need to be interconnected somehow to enable the content exchange. More specifically, such content exchange is preferably such that, by simply manually compressing the one or the other element, fluid is urged to flow from the one element into the other element. For instance, two adjacent elements may be interconnected, e.g., glued together, in a certain area and there may be integrated in such area at least one pressure relief valve which opens at a predetermined pressure. This way, the overall structure can be held relatively simple. More advantageously, the pressure relief valve is a two-way pressure relief valve that opens in the one direction, or the other direction, depending on the side where the predetermined pressure is applied.

Instead of inter-exchanging fluid directly between the first and second elements, an intermediate reservoir may be interposed between the two elements, such that fluid can be exchanged between the intermediate reservoir and the first and second elements, so as to change their respective content. In other words, fluid squeezed out of one element into the intermediate reservoir will cause an equivalent amount of fluid to be urged from the intermediate reservoir into the respective other element. The intermediate reservoir may be arranged, e.g., in a rigid back plate of the breast implant described before.

So far, embodiments have been described wherein the shape of the breast can be changed, e.g., from flat to high, but the size, i.e., the volume of the breast implant, remains constant. According to an even further preferred embodiment, however, the volume of the breast implant can also be changed. For this purpose, at least one reservoir different from the lubricating fluid reservoir is provided in fluid connection with at least one element contained in the casing, such that fluid can be exchanged between this (further) reservoir and the respective element. The further reservoir may be adapted for implantation below the minor pectoralis muscle, or between the major and the minor pectoralis muscles, similar to the lubricating fluid reservoir. It may be placed even more remotely from the breast implant in which case, again, a conduit will have to be provided for the fluid exchange. In addition, a servo system may be added to facilitate the fluid exchange between the further reservoir and the respective element.

In this context, the term “element” may include a chamber having a flexible outer wall and being filled with a fluid, in particular a liquid, and being specifically adapted for supplying fluid to and removing fluid from such chamber in order to ultimately change the size and/or shape of the overall breast implant. The term “element”, in this case, may likewise cover a plurality of permanently interconnected chambers. However, where the element or elements have a constant volume, i.e., not being adapted for exchanging fluid content, the content of the elements need not necessarily be a fluid but may likewise be granular. It is not even necessary in such case that the elements have a flexible outer wall. For instance, a great number of small elements, such as 100 or more, may be contained in the casing of the breast implant system, as already mentioned before. These small elements may or may not be entirely stiff. If these elements are contained in a casing of the breast implant according to the previously described preferred embodiment, their purpose is to fill the casing and impart on the casing a particular outer shape which, in turn, defines the outer shape of the breast implant. Thus, by changing the volume of the one or more elements within the casing, and/or by rearranging the element or elements within the casing, the shape and/or size of the breast implant will be affected accordingly.

As previously mentioned, the use of a servo system may facilitate the exchange of fluid between the lubricating fluid reservoir and the casing (and likewise between a “further” reservoir and one or more of the elements contained in the casing). A servo system, in the sense of the present invention, is to be understood as a system in which an amount of fluid is displaced between sub-chambers of the lubricating fluid reservoir, which amount is different to the amount of fluid exchanged between the lubricating fluid reservoir and the casing (or the “further” reservoir and one or more of the elements contained in the casing).

There are various alternative ways of realizing such servo system in the breast implant system of the present invention. In this context, it is preferable to provide a spring element to urge the lubricating fluid reservoir, or at least one of the sub-chambers thereof, into a state of minimum or maximum volume, i.e., into a normally small or a normally large state. Energy, such as a manual compressing force on a subcutaneously implanted sub-chamber, is then only needed to exchange fluid between the casing and the reservoir in one direction, whereas the necessary force required to exchange the fluid in the opposite direction is provided by the spring force. In the same manner, fluid can be exchanged between a further reservoir and one or more elements contained in the casing, in order to change the size and/or shape of the breast implant.

The servo system can be designed as a reverse servo system, to the extent that only a small amount of fluid needs to be exchanged between the sub-chambers of the reservoir in order to achieve a relatively large amount of fluid exchange between the reservoir and the casing. This means that a relatively large force, but small stroke, is needed to achieve the relatively large amount of fluid exchange between the reservoir and the casing. This is particularly convenient where one of the sub-chambers of the reservoir is provided for subcutaneous implantation to be manually compressible by the patient from outside the patient's body. Thus, the subcutaneously arranged compressible sub-chamber may have a relatively small volume, and will therefore not adversely affect the patient's visual appearance, however, with the negative side effect that the patient will have to apply a relatively large force to the relatively small subcutaneous sub-chamber in order to achieve the desired, relatively large fluid exchange. Such sub-chamber can be placed advantageously under the patient's arm.

With time, it may become necessary to add lubricating fluid to the reservoir, or to add to a further reservoir fluid used for inflating and deflating one or more elements contained in the casing. In particular, when gas is contained in one or another reservoir, it is possible that part of the gas will escape over time due to leakage. Therefore, in order to maintain a desired balance in the reservoir or reservoirs, the breast implant system, according to a preferred embodiment, includes at least one injection port for implantation under the patient's skin, so as to allow fluid to be added to, or removed from, any reservoir by injection from outside the patient's body. Thus, the injection port is primarily provided for calibrating purposes. Preferably, the system is adapted to maintain shape and size of the breast implant while fluid is added to or removed from the reservoir via said injection port. Then, after fluid has been added or withdrawn from said injection port, the breast implant is adapted to change shape or size to a larger or lesser extent in a calibration procedure.

The injection port or ports can further be used to adjust the pressure in the breast implant system. For instance, when a patient has achieved a particular distribution of fluid among fluid-filled elements contained in the casing, it is convenient for the patient to release any excess pressure from the system by selectively removing fluid from the system through the injection port or ports.

A pump may be provided in the breast implant system for exchanging lubricating fluid between the reservoir and the casing. The same pump, or a different pump, may likewise be used for pumping fluid between fluid-filled elements contained in the casing, or for pumping fluid into and out of one or more fluid-filled elements contained in the casing. The further reservoir may alternatively be connected to the pump, so as to allow for fluid exchange between fluid-filled elements in the casing by pumping fluid with said pump, from a first element into the further reservoir, and from the further reservoir into the second element, and vice versa.

The pump or pumps may be adapted for implantation under the patient's skin, to be manually operable through the skin. In this case, a purely hydraulic or purely pneumatic pump may be used.

However, where the pump is not manually operable, the breast implant system may comprise at least one motor for automatically driving the pump. In this case, the pump may be of the hydraulic, pneumatic, or mechanical type. In addition, a manually operable switch for activating the motor may be arranged subcutaneously for operation by the patient from outside the patient's body. The motor itself may be arranged to be driven by electric or electromagnetic energy, by an electric or magnetic pulsating field, or by ultrasonic energy.

The breast implant system may further comprise an energy source for supplying the energy, directly or indirectly, to at least one energy consuming part of the system, in particular, to the aforementioned motor for driving the pump. Such energy source may include energy storage means, such as a battery or an accumulator, in particular one or more of a rechargeable battery and a capacitor. The energy source, when provided outside the patient's body, preferably comprises a wireless energy transmitter for wirelessly transmitting energy from outside the patient's body, directly or indirectly, to the energy consuming part, or to an implanted energy storage means.

The breast implant system preferably further comprises an implantable energy-transforming device for transforming wirelessly transmitted energy into electric energy. The electric energy is stored in the energy storage means, and/or is used to drive the energy consuming part, such as the motor and the pump, as the energy-transforming device transforms the wireless energy into the electric energy. Alternatively, the energy consuming part may be adapted to directly transform the wirelessly transmitted energy into kinetic energy.

It is further preferred to provide the breast implant system with a control unit to directly or indirectly control one or more elements of the breast implant system, in particular the pump and the motor. For instance, the control unit may be primarily adapted to control the exchange of lubricating fluid between the reservoir and the casing, and/or to control fluid flow into, out of, or between one or more elements contained in the casing. Preferably, such controlling action is carried out non-invasively from outside the patient's body, such as by wireless remote control. In this case, a part of the control unit is implanted in the patient's body, whereas another part is not implanted. In particular, in the case where the control unit is completely implanted in the patient's body, a manually operable switch for activating the control unit may be arranged subcutaneously to be operable from outside the patient's body.

Where one part of the control unit is provided outside the patient's body, and the other part is implanted in the patient's body, the external part of the control unit may be used to program the implanted part of the control unit, preferably wirelessly. In addition, the implantable part of the control unit may be adapted to transmit feedback signals to the external part of the control unit. Such signals may relate to: functional and/or physical parameters of the system, and/or patient; and/or may relate to the energy stored in the energy storage means; and/or to an energy balance of the system.

### N: Treating Obesity and/or Reflux

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for treating obesity and/or reflux, such as volume filling devices, stretching devices, and/or movement restriction devices. Examples of such devices for treating obesity and/or reflux will now be described.

According to one embodiment, an apparatus comprising at least one volume filling device adapted to be at least substantially invaginated by a stomach wall portion of the patient is provided.

The volume filling device may be adapted to be placed outside of the stomach wall with the outer surface of the volume filling device resting against the outside of the stomach wall, such that the volume of the food cavity is reduced in size by a volume substantially exceeding the volume of the volume filling device. The volume filling device may have a maximum circumference of at least 30 millimeters and may have a volume of less than 0.0002 m³ whereby an apparatus for treating obesity is obtained.

By invaginating a volume filling device by the stomach wall on the outside thereof this device is protected from the stomach acids and will thus remain functioning for a very long time.

Preferably, the at least one volume filling device is adapted to be at least substantially invaginated by a stomach wall portion of the patient by suturing a first part of a stomach wall serosa towards a second, different part of a stomach wall serosa, the serosa being the outer layer of the stomach wall.

The stomach is mounted in two sails extending on the lesser and greater curvature of the stomach, called the greater or majus omentum and lesser or minus omentum, respectively. The lesser omentum is also called the hepatogastric omentum including the hepatogastric ligament. Preferably, the volume filling device has a shape adapted for permitting the device to be invaginated without penetrating the hepatogastric ligament of the patient.

Preferably, the volume filling device has an outer surface that includes a biocompatible material.

Preferably, the volume filling device is adapted to be completely invaginated by the stomach wall of the patient and to be placed outside the stomach wall via a gastroscopic instrument. To this end the volume filling device may comprise an attachment device adapted to co-operate with a gripping instrument. Suitably, the volume filling device is adapted to be non-invasively adjustable postoperatively.

The apparatus may comprise a fixation device, suitably two or more fixation devices, adapted to be involved in the fixation of the volume filling device to the stomach wall. The volume filling device may comprise a holding device adapted to be held by an instrument, suitably two or more holding devices, to simplify the implantation of the device.

At least a part of the volume filling device may be made of a material which is not destructible by stomach acid. The volume filling device may be destructible by acids, for example hydrochloric acid.

In an embodiment, the volume filling device is inflatable to an expanded state and comprises an enclosure wall defining a chamber, wherein the volume filling device is inflated with a gel or fluid supplied into the chamber. At least one tube may be connected to the volume filling device for supplying gel or fluid to the chamber. An injection port connectible with the tube may be provided. Alternatively, the volume filling member may be provided with an inlet port for a fluid or a gel connectible to a gastroscopic instrument, wherein the inlet port comprises a fluid connection adapted to interconnect the inflatable device and the gastroscopic instrument.

The volume filling device may include a homogenous material, such as gel having a shore value of less than 15. The device may also be a solid body.

The volume filling device may comprise a rigid, elastic or flexible outer surface. Where the outer surface is rigid, it is rigid enough to maintain non-deformed when subject to forces created by stomach movements. The volume filling device may comprise a flexible non-elastic material.

In accordance with a first general design of the volume filling device, the device has a maximum circumference as seen in a plane perpendicular to an axis through the device. The circumferences of the device as seen in other planes perpendicular to said axis are equal to the maximum circumference or decrease as seen along said axis in the direction from the maximum circumference. For example, the device may be substantially egg shaped, spherically shaped, or substantially shaped like an egg with an indented middle section or like a bent egg.

In accordance with a second general design of the device, the circumference of the device as seen in a plane perpendicular to an axis through the device increases and decreases at least two times as the plane is displaced along said axis, or decreases and increases at least one time as the plane is displaced along said axis. For example, the device may be substantially shaped like a kidney.

The volume filling device preferably has an elongated, rounded, bent and/or curved shape.

The volume filling device has a circumference of at least 60, 90, 120, 150, 180 or 220 mm.

By providing a volume filling device with a volume of less than 0.0002 m³, several advantages are obtained. Such a volume filling device is suitable for invagination with a good result between the lesser and greater curvatures, including sails with blood vessels and nerves, holding the stomach in place. The lesser and greater curvatures have a mutual distance in the magnitude of 1 decimeter or slightly more. Taking into account that the circumference of the device is 3.14 times the diameter, the full distance between the lesser and greater curvature is occupied at small sizes of the volume filling device. With current biocompatible material it is more or less impossible to keep a gas inside the volume filling device without losing gas to the surroundings. Thus, the volume filling device is suitably filled with a fluid, giving it substantial weight at large volumes. A volume filling device comprised in the inventive apparatus, having a volume of less than 0.0002 m³, can be carried by the stomach wall without any problems.

The volume filling device may comprise at least two interconnectable portions adapted to be placed inside the stomach as separate portions.

The volume filling device may comprise an elastic or flexible material, a bio-compatible material and/or silicone.

Suitably, the volume filling device is provided with at least one layer. For example, a metal layer, a Parylene layer, a polytetrafluoroethylene layer or a polyurethane layer. The layers may comprise multiple layers in any order. Suitably, one of the layers may be made of made of metal, silicon or PTFE. The volume filling device may comprise an outer surface layer of polyurethane, Teflon®, or polytetrafluoroethylene, or a combination thereof.

The volume filling device may comprise a fluid adapted to be transformed into solid state or fixed form. Such a fluid may be liquid polyurethane or iso-tonic. The fluid may comprises large molecules, such as iodine molecules, to prevent diffusion.

Suitably, the volume filling device is deformable to a maximum diameter, so as to be insertable into a laparoscopic trocar.

Preferably, the volume filling device is adapted to be kept in place by stomach-to-stomach sutures or staples to invaginate the device in the stomach wall. Advantageously, the volume filling device has varying circumference to better be kept in place invaginated in the stomach wall of the patient. The stomach-to-stomach sutures or staples may be provided with fixation portions exhibiting a structure adapted to be in contact with the stomach wall to promote growth in of human tissue to secure long term placement of the volume filling device attached to the stomach wall. The structure may comprise a net like structure.

In embodiment of the invention, the apparatus comprises a stretching device placed outside the stomach wall and adapted to stretch a portion of the stomach wall, thereby affecting the patient's appetite. Where the volume filling device is inflatable, the apparatus may comprise a fluid connection interconnecting the stretching device and the volume filling device.

In an embodiment, the apparatus comprises a stretching device comprising at least one operable stretching device implantable in an obese patient and adapted to stretch a portion of the patient's stomach wall and an operation device for operating the stretching device when implanted to stretch the stomach wall portion such that satiety is created.

In an embodiment, the apparatus comprises at least one operable stretching device implantable in the patient and adapted to stretch a portion of the patient's stomach wall, and an implantable control unit for automatically controlling the operable stretching device, when the control unit and stretching device are implanted, to stretch the stomach wall portion in connection with the patient eating such that satiety is created.

In an embodiment, the apparatus comprises a stretching device comprising at least one operable stretching device implantable in an obese patient and adapted to stretch a portion of the patient's stomach wall, wherein said stretching device comprising an expandable stretching reservoir and an operation device for operating the stretching device when implanted to stretch the stomach wall portion, wherein the volume filling device is inflatable and in fluid connection with said stretching reservoir, wherein said operation device comprises a pump for pumping fluid between said main reservoir and said stretching reservoir to stretch said stomach wall portion such that satiety is created. A control device may be provided for controlling said stretching device including said pump. The control device may comprise a wireless remote control adapted to control the stretching device from the outside of the patient's body, or an implantable control unit for controlling said stretching device. Alternatively, the control device may comprise a subcutaneously placed switch or reservoir adapted to control the stretching device from the outside of the patient's body. A sensor or sensing device to be implanted in the patient body may be provided, wherein the implantable control unit is adapted to control the stretching device from the inside of the patient's body using information from said a sensor or sensing device, adapted to sense, direct or indirect, the food intake of the patient.

In an embodiment, the volume filling device comprises a main volume filling reservoir, a stretching device comprising at least one operable stretching device implantable in an obese patient and adapted to stretch a portion of the patient's stomach wall, wherein said stretching device comprising an expandable reservoir, adapted to be invaginated in the stomach wall at the upper part of the stomach, higher up than the inflatable main volume filling device when the patient is standing, wherein the volume filling device is inflatable and in fluid connection with said stretching reservoir, wherein normal contractions of the stomach wall, related to food intake, cause fluid to flow from said invaginated main volume filling reservoir lower placed onto the stomach wall adapted to cause said stretching reservoir to stretch said stomach wall portion such that satiety is created. The fluid connection between the main volume filling device reservoir and the stretching reservoir comprises a non-return valve. The fluid connection between the main volume filling device reservoir and the stretching reservoir comprises a release function adapted to release the volume in the stretching reservoir back to the main volume filling device reservoir. Said release function may comprise a fluid return connection of a substantially smaller area than said fluid connection, to slowly release back fluid to said main volume filling device reservoir from the stretching reservoir to release said stretching of the stomach wall portion. A further manual control device comprising a subcutaneously placed reservoir adapted to control the stretching device from the outside of the patient's body may be provided to further affect the stretching device to stretch the stomach wall portion.

In an embodiment, the a main volume filling device reservoir adapted to be inflatable may be provided, wherein the volume filling device further comprises an expandable structure, adapted to expand, when the device is invaginated in the stomach wall, wherein said structure comprising a bellow adapted to take into account the fibrosis surrounding the device when implanted, such that the movement of the bellow is substantially un-affected of said fibrosis.

In an embodiment, the apparatus comprises a stretching device comprising at least one operable stretching device implantable in an obese patient and adapted to stretch a portion of the patient's stomach wall and wherein the stretching device comprising an expandable structure, adapted to expand and stretch the stomach wall portion, when the device is invaginated in the stomach wall, wherein said structure comprising a special bellow adapted to take into account the fibrosis surrounding the device when implanted, such that the movement of the bellow is substantially un-affected of said fibrosis. An operation device for operating the stretching device may be provided to stretch the stomach wall portion such that satiety is created. The apparatus may comprise an implantable control unit for automatically controlling the operable stretching device, when the control unit and stretching device are implanted, to stretch the stomach wall portion in connection with the patient eating such that satiety is created.

In an embodiment, the apparatus comprises a stretching device comprising at least one operable stretching device implantable in an obese patient and adapted to stretch a portion of the patient's stomach wall such that satiety is created. The control device may comprise a wireless remote control adapted to control the stretching device from the outside of the patient's body or an implantable control unit for controlling said stretching device. Alternatively, said control device may comprise a subcutaneously placed switch or reservoir adapted to control the stretching device from the outside of the patient's body. A sensor or sensing device adapted to be implanted in the patient body may be provided, wherein the implantable control unit is adapted to control the stretching device from the inside of the patient's body using information from said sensor or sensing device, adapted to sense, direct or indirect, the food intake of the patient.

In an embodiment, the apparatus is further adapted to treat reflux disease. To this end, it further comprises an implantable movement restriction device adapted to be at least partly invaginated by the patient's stomach fundus wall and having an outer surface that includes a biocompatible material, wherein a substantial part of the outer surface of the movement restriction device is adapted to rest against the stomach wall without injuring the latter in a position between the patient's diaphragm and at least a portion of the lower part of the invaginated stomach fundus wall, such that movement of the cardiac notch of the patient's stomach towards the patient's diaphragm is restricted, when the movement restriction device is invaginated, to thereby prevent the cardia from sliding through the patient's diaphragm opening into the patient's thorax, so as to maintain the supporting pressure against the patient's cardia sphincter muscle exerted from the patient's abdomen, the movement restriction device having a size of at least 125 mm3 and a circumference of at least 15 mm.

In another embodiment, the apparatus is further adapted to treat reflux disease. To this end, it further comprises an implantable movement restriction device having an outer surface including a biocompatible material, wherein the movement restriction device is adapted to rest with at least a part of its outer surface against the patient's stomach fundus wall, in a position between the patient's diaphragm and the fundus wall, such that movement of the cardiac notch of the patient's stomach towards the patient's diaphragm is restricted, when the movement restriction device is implanted in the patient, to thereby prevent the cardia from sliding through the patient's diaphragm opening into the patient's thorax, so as to maintain the supporting pressure against the patient's cardia sphincter muscle exerted from the patient's abdomen, wherein the movement restriction device having a size of at least 125 mm3 and a circumference of at least 15 mm, and an a fixation device adapted to secure the movement restriction device in said position, when the movement restriction device is implanted.

In another embodiment, the apparatus is further adapted to treat reflux disease. To this end, it further comprises an implantable movement restriction device adapted to be at least partly invaginated by the patient's stomach fundus wall and having an outer surface that includes a biocompatible material, wherein a substantial part of the outer surface of the movement restriction device is adapted to rest against the stomach wall without injuring the latter in a position between the patient's diaphragm and at least a portion of the lower part of the invaginated stomach fundus wall, such that movement of the cardiac notch of the patient's stomach towards the patient's diaphragm is restricted, when the movement restriction device is invaginated, to thereby prevent the cardia from sliding through the patient's diaphragm opening into the patient's thorax, so as to maintain the supporting pressure against the patient's cardia sphincter muscle exerted from the patient's abdomen, the movement restriction device having a size of at least 125 mm3 and a circumference of at least 15 mm, further comprising a stretching device comprising at least one operable stretching device implantable in the obese patient and adapted to stretch a portion of the patient's stomach wall such that satiety is created.

In another embodiment, the apparatus is further adapted to treat reflux disease. To this end, it further comprises an implantable movement restriction device having an outer surface including a biocompatible material, wherein the movement restriction device is adapted to rest with at least a part of its outer surface against the patient's stomach fundus wall, in a position between the patient's diaphragm and the fundus wall, such that movement of the cardiac notch of the patient's stomach towards the patient's diaphragm is restricted, when the movement restriction device is implanted in the patient, to thereby prevent the cardia from sliding through the patient's diaphragm opening into the patient's thorax, so as to maintain the supporting pressure against the patient's cardia sphincter muscle exerted from the patient's abdomen, wherein the movement restriction device having a size of at least 125 mm3 and a circumference of at least 15 mm, and a fixation device adapted to secure the movement restriction device in said position, when the movement restriction device is implanted, further comprising a stretching device comprising at least one operable stretching device implantable in the obese patient and adapted to stretch a portion of the patient's stomach wall such that satiety is created.

In an embodiment, the apparatus further comprises a stretching device comprising three or more mechanical parts engaged with different parts of the stomach wall, one part each, wherein said engagement includes suturing or stapling to the stomach wall or invaginating the mechanical parts in the stomach wall part with stomach to stomach sutures, wherein the three or more mechanical parts are adapted to move in relation to each other adapted to stretch three different wall portions, the stretching device further adapted to having said wall portions stretched independently from each other both regarding force used for stretching the stomach wall portion as well as, time periods the stretching is applied, and when the stretching is applied.

In an embodiment, the apparatus further comprises a stretching device comprising two or more hydraulic parts engaged with different parts of the stomach wall, one part each, wherein said engagement includes suturing or stapling to hydraulic part to the stomach wall or invaginating the hydraulic parts in the stomach wall part, with stomach to stomach sutures, wherein the two or more hydraulic parts are adapted to move in relation to each other adapted to stretch three different wall portions, the stretching device further adapted to having said wall portions stretched independently from each other both regarding force used for stretching the stomach wall portion as well as, time periods the stretching is applied, and when the stretching is applied.

In an embodiment, the apparatus further comprises a stretching device is engaged with a part of the stomach wall, including suturing or stapling the stretching device to the stomach wall or invaginating the stretching device in the stomach wall part, with stomach to stomach sutures, wherein the stretching device is further adapted to stretch a stomach wall portion controlling force used for stretching the stomach wall portion as well as, time periods the stretching is applied, and when the stretching is applied.

In an embodiment, the apparatus further comprises a stretching device comprising two parts engaged with different parts of the stomach wall, one part each, wherein said engagement includes suturing or stapling the parts to the stomach wall or invaginating the parts in the stomach wall part, with stomach to stomach sutures, wherein the stretching device further adapted to have different wall portions stretched independently from each other controlling force used for stretching the stomach wall portion as well as, time periods the stretching is applied, and when the stretching is applied.

In an embodiment, the apparatus further comprises an external control unit for controlling the volume filling device from the outside of the patient's body. The external control unit may comprise a wireless remote control adapted to control the device from the outside of the patient's body. Alternatively, the external control unit may comprise a subcutaneously placed switch or reservoir adapted to control the device from the outside of the patient's body.

In an embodiment, the apparatus further comprises a sensor or sensing device adapted to be implanted in the patient body, wherein the implantable control unit is adapted to control the device from the inside of the patient's body using information from said a sensor or sensing device, adapted to sense, direct or indirect, the food intake of the patient.

In accordance with another aspect of the present invention, there is provided an apparatus for treating obesity of an obese patient having a stomach with a food cavity, the apparatus comprising at least one volume filling device adapted to be at least substantially invaginated by a stomach wall portion of the patient and having an outer surface that includes a biocompatible material, wherein the volume filling device is adapted to be placed inside the stomach with the outer surface of the volume filling device resting against the inside of the stomach wall, such that the volume of the food cavity is reduced in size by a volume substantially exceeding the volume of the volume filling device, the volume filling device having a maximum circumference of at least 30 millimeters.

In a preferred embodiment, the system comprises at least one switch implantable in the patient for manually and non-invasively controlling the apparatus.

In another preferred embodiment, the system comprises a wireless remote control for non-invasively controlling the apparatus.

In a preferred embodiment, the system comprises a hydraulic operation device for operating the apparatus.

In one embodiment, the system comprises a motor or a pump for operating the apparatus.

### O, P: Egg and/or Embryo Control of a Female Patient to Avoid or Promote Pregnancy

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for pregnancy control of a female patient. The control unit/controller, control systems, communication systems, housing/fixation units and methods could also be used for controlling, communicating with and/or operating a device for embryo control of a female patient. Examples of such devices for pregnancy/embryo control of a female patient will now be described.

The object of the present invention is to provide a system and a method for pregnancy control and/or embryo of a female patient.

The inventive system allows the egg from the ovary to be retained in the oviduct to promote or to avoid pregnancy. When it is sought for promoting pregnancy, the inventive system allows the egg from the ovary to be retained in the oviduct for a couple of days to be able to achieve the right timing for egg release and thereby increasing the likelihood to get pregnant with up to 10 times. A respective (restriction) device is placed on the two oviducts to restrict and release the oviduct, thereby effecting the above mentioned retaining of the egg.

In accordance with a first aspect of the present invention, there is provided a system for treating a female patient to avoid or promote pregnancy comprising a (restriction) device adapted to postoperatively restrict and release an oviduct of the patient. To reduce any possible risk of the egg being to the wall of the oviduct the restriction device may be combined with a movement device. Such a device may comprise a vibrating device or a stimulating device. The stimulation device may create a peristaltic like wave in the oviduct. For example, a vibrating device may be provided, which causes vibrations in a hydraulic fluid or mechanically causes vibrations in the wall of the oviduct. Another embodiment is an electrical stimulation device causing contractions or stimulation waves in the upstream direction of the oviduct. The device may also be replaced by a device not permitting the flow of the egg down to the uterus but without any full restriction. This may be created by using a device causing a peristaltic like wave in the oviduct in the direction of the ovary, thus without fully closing the oviduct instead preventing further transport of the egg down to the uterus by the wave, but still allowing the sperm to reach the egg. Any possible device without restriction at all or in any combinations of partly restriction or vibration or stimulation may be used. This will then allow the egg to be accumulated in the oviduct and allowing conception to take place over a longer period of time.

In accordance with a second aspect of the invention, there is provided a method of avoiding or promoting pregnancy of a female patient, comprising the steps of restricting an oviduct of the patient to provide a restriction to accumulate at least one egg released from the ovary in the oviduct for a predetermined period of time, and releasing the restriction to admit the at least on egg in the oviduct to be transported to the uterus.

All embodiments and features described below may if possible be used for both, be adapted to be used with the apparatus, and being used with any of the methods described below.

A system for treating a female patient to avoid or promoting pregnancy comprising; a (restriction) device adapted to postoperatively restrict and release an oviduct of the patient.

The restriction device is preferably adapted to provide a restriction of the oviduct to accumulate at least one egg released from the ovary in the oviduct.

The restriction device may be adapted to provide a release of the oviduct, after accumulating the egg normally up to three days, when pregnancy is wanted or not possible to achieve right timing for pregnancy.

The restriction device may be adapted to be adjusted from outside the patient's body to restrict and release the oviduct passageway, preferable adjusted from outside the patient's body non-invasively.

It may also be adjusted by manual manipulation or adapted to be adjusted by electrical or magnetic power or adapted to be adjusted by hydraulic power. The hydraulic power may comprise at least one subcutaneously placed reservoir controlled by the patient.

The restriction device may of course preferable be adapted to be adjusted reversible.

The system is adapted to provide a restriction to accumulate at least one egg released from the ovary in the oviduct for a predetermined period of time, and releasing the restriction when convenient for the patient to avoid pregnancy or increase the likelihood to get pregnant. To avoid pregnancy, the predetermined period of time may be between 1 and 30 days or a predetermined period of time more than 30 days. To promote pregnancy, the predetermined period of time is adapted to be between 0 and 2 days repeated every month until pregnancy is achieved or preferable a predetermined period between 8 and 12 hours.

### Flow Restriction

The system of the present invention is well suited for the controlling the flow of eggs into the uterus of a female patient. Basically, the (restriction) device could be performed in eight different principle ways:
Hydraulic restrictions
Mechanical restrictions
Combination of any hydraulic or mechanical restriction device with a stimulation device for restricting.
Stimulation device alone (for restricting).
Vary the restriction area. Any of these in any combination could be used to over time vary the restriction area of the oviduct from one portion to another one and later back again. Several different areas could be involved in such a system to vary the restriction portion/area. This would allow the oviduct to recover the restricted area still keeping the restriction over a longer period.
Using a moving device. When the restriction area being moved between different areas, specially when the restriction is moved upstream towards the ovary, it may be convenient to use a moving device, to avoid any egg being squeezed in a new upstream restriction area and by mistake being released for further downstream transportation when the restriction being released and moved further upstream. The moving device may be adapted to create a movement of the oviduct wall to create a movement of any egg placed in the new upcoming restricted area. This could be caused by any of the above-mentioned restriction devices, mechanical, hydraulic or stimulation device alone or in any combination, but may also be a separate device which also may be a mechanical, a hydraulic or a stimulation device. In one embodiment the moving device cause vibrations.
Peristaltic like wave movement of the oviduct wall towards the ovary. Another principle is to stop the egg reaching the uterus by creating peristaltic like wave movements of the oviduct wall.

Such peristaltic like wave movement in the upstream direction towards the ovary may stop flow of an egg towards the uterus preferable always fully restricting some part of the oviduct to avoid any sperm to pass. Such peristaltic wave like movements upstream direction towards the ovary may instead stop flow of any egg towards the uterus without fully restricting the oviduct to allow any sperm to pass and also
Peristaltic like wave movement of the oviduct wall towards the uterus by being able to cause movement of the egg down to uterus with a downstream peristaltic wave. Another principle is to promote the egg reaching the uterus by being able to cause movement of the egg down to uterus with a downstream peristaltic wave when risk of pregnancy does not occur.

The areas will be outlined below:

### Hydraulic Restriction

Where the operation device hydraulically operates the constriction device of the restriction or combined restriction/stimulation unit (d), it includes hydraulic means for adjusting the (restriction) device.

In an embodiment of the invention, the hydraulic means comprises a reservoir and an expandable/contractible cavity in the constriction device, wherein the operation device distributes hydraulic fluid from the reservoir to expand the cavity and distributes hydraulic fluid from the cavity to the reservoir to contract the cavity. The cavity may be defined by a balloon of the constriction device that abuts the tissue wall portion of the patient's organ, so that the patient's wall portion is constricted upon expansion of the cavity and released upon contraction of the cavity.

Alternatively, the cavity may be defined by a bellows that displaces a relatively large contraction element of the constriction device, for example a large balloon that abuts the wall portion, so that the patient's wall portion is constricted upon contraction of the bellows and released upon expansion of the bellows. Thus, a relatively small addition of hydraulic fluid to the bellows causes a relatively large increase in the constriction of the wall portion. Such a bellows may also be replaced by a suitably designed piston/cylinder mechanism.

Where the hydraulic means comprises a cavity in the constriction device, the apparatus can be designed in accordance with the options listed below.
1) The reservoir comprises first and second wall portions, and the operation device displaces the first and second wall portions relative to each other to change the volume of the reservoir, such that fluid is distributed from the reservoir to the cavity, or from the cavity to the reservoir. 1a) The first and second wall portions of the reservoir are displaceable relative to each other by at least one of a magnetic device, a hydraulic device or an electric control device.
2) The apparatus comprises a fluid conduit between the reservoir and the cavity, wherein the reservoir forms part of the conduit. The conduit and reservoir and apparatus are devoid of any non-return valve. The reservoir forms a fluid chamber with a variable volume and distributes fluid from the chamber to the cavity by a reduction in the volume of the chamber and withdraws fluid from the cavity by an expansion of the volume of the chamber. The apparatus further comprises a motor for driving the reservoir, comprising a movable wall of the reservoir for changing the volume of the chamber.

In a special embodiment of the invention, the operation device comprises a reverse servo operatively connected to the hydraulic means. The term “reverse servo” is to be understood as a mechanism that transfers a strong force acting on a moving element having a short stroke into a weak force acting on another moving element having a long stroke; i.e., the reverse function of a normal servo mechanism. Thus, minor changes in the amount of fluid in a smaller reservoir could be transferred by the reverse servo into major changes in the amount of fluid in a larger reservoir. The reverse servo is particularly suited for manual operation thereof.

Preferable a manually operated reservoir could be placed subcutaneously for manual manipulation thereof.

### Mechanical Restriction

Where the operation device mechanically operates the (restriction) device or the restriction/stimulation unit (d), it may be non-inflatable. Furthermore, the operation device may comprise a servo system, which may include a gearbox. The term “servo system” encompasses the normal definition of a servo mechanism, i.e., an automatic device that controls large amounts of power by means of very small amounts of power, but may alternatively or additionally encompass the definition of a mechanism that transfers a weak force acting on a moving element having a long stroke into a strong force acting on another moving element having a short stroke. Preferably, the operation device operates the constriction device in a non-magnetic and/or non-manual manner. A motor may be operatively connected to the operation device. The operation device may be operable to perform at least one reversible function and the motor may be capable of reversing the function.

### Combination of Mechanical or Hydraulic (Restriction) Device and a Stimulation Device

The present invention provides an advantageous combination of restriction and stimulation devices, which results in a two-stage influence on the flow of eggs in the lumen of the oviduct. Thus, the constriction device may gently constrict the tissue wall of the oviduct wall by applying a relatively weak force against the wall portion, and the stimulation device may stimulate the constricted wall portion to achieve the desired final influence on the flow in the lumen. The phrase “gently constricting a portion of the tissue wall” is to be understood as restricting the wall portion without substantially hampering the blood circulation in the tissue wall.

In accordance with a first flow restriction option, the control device controls the constriction device to constrict the wall portion, such that flow in the lumen is restricted or stopped, and controls the stimulation device to stimulate the constricted wall portion to cause contraction thereof, such that flow in the lumen is further restricted or more safely stopped. More precisely, a control device may control the stimulation device in a first mode to stimulate the constricted wall portion to further restrict or stop the flow in the lumen and to:
a) control the stimulation device in a second mode to cease the stimulation of the wall portion to increase the flow in the lumen; or
b) control the stimulation and constriction devices in the second mode to cease the stimulation of the wall portion and release the wall portion to restore the flow in the lumen.

Thus both a method for controlling the flow in the lumen and an apparatus adapted to control the flow in the lumen may be implemented according to different embodiments and features in any combination described in this document.
d) Stimulation device alone, which could both 1) restrict by stimulation and 2) also creating peristaltic wave like movements to if a) upstream stop flow without fully restricting the oviduct and also b) being able to cause movement of the egg down to uterus with a downstream peristaltic wave:

Preferably, the stimulation device is adapted to stimulate different areas of the wall portion as the (restriction) device restricts the wall portion, and the control device controls the stimulation device to intermittently and individually stimulate the areas of the wall portion. This intermittent and individual stimulation of different areas of the wall portion of the oviduct allows tissue of the wall portion to maintain substantially normal blood circulation during the operation of the apparatus of the invention.

The stimulation of different areas may be used both for closing the lumen in a safe way but also be used for creating a peristaltic wave in the oviduct.

### Movement of the Egg in the Oviduct Lumen

In one embodiment the (restriction) device is adapted to constrict the wall portion to restrict or vary the flow in the lumen, and the control device controls the stimulation device to progressively stimulate the constricted wall portion, in the downstream or upstream direction of the lumen, to cause progressive contraction of the wall portion to move the egg downstream in the lumen or prevent further transport down of the egg to the uterus.

### Design of Control Device

The (restriction) device is preferable controlled from outside the body in a manual way. The (restriction) device may also be powered. A control device may be supplied. The control device suitably controls the (restriction) device or the stimulation device or restriction/stimulation unit from outside the patient's body. Preferably, the control device is operable by the patient. For example, the control device may comprise a manually operable switch for switching on and off the constriction/stimulation unit, wherein the switch is adapted for subcutaneous implantation in the patient to be manually or magnetically operated from outside the patient's body. Alternatively, the control device may comprise an external control unit in the form of a hand-held wireless remote control, which is conveniently operable by the patient to switch on and off the constriction/stimulation unit. The wireless remote control may also be designed for application on the patient's body like a wristwatch. Such a wristwatch type of remote control may emit a control signal that follows the patient's body to implanted signal responsive means of the apparatus.

In a preferred embodiment of the invention, the constriction device is adjustable to enable adjustment of the constriction of the wall portion as desired, wherein the control device controls the constriction device to adjust the constriction of the wall portion. The control device may control the constriction and stimulation devices independently of each other, and simultaneously. Optionally, the control device may control the stimulation device to stimulate, or to not stimulate the wall portion while the control device controls the constriction device to change the constriction of the wall portion.

Initially, the constriction device may be calibrated by using the control device to control the stimulation device to stimulate the wall portion, while controlling the constriction device to adjust the constriction of the wall portion until the desired restriction of the flow in the lumen is obtained.

Regarding Stimulation Alone or in Combination with a (Restriction) Device

The control device may control the stimulation device to stimulate one or more of the areas of the wall portion at a time, for example by sequentially stimulating the different areas. Furthermore, the control device may control the stimulation device to cyclically propagate the stimulation of the areas along the wall portion, preferably in accordance with a determined stimulation pattern. To achieve the desired reaction of the tissue wall during the stimulation thereof, the control device may control the stimulation device to, preferably cyclically, vary the intensity of the stimulation of the wall portion.

In a preferred embodiment of the invention, the control device controls the stimulation device to intermittently stimulate the areas of the wall portion with pulses that preferably form pulse trains. At least a first area and a second area of the areas of the wall portion may be repeatedly stimulated with a first pulse train and a second pulse train, respectively, such that the first and second pulse trains over time are shifted relative to each other. For example, the first area may be stimulated with the first pulse train, while the second area is not stimulated with said second pulse train, and vice versa. Alternatively, the first and second pulse trains may be shifted relative to each other, such that the first and second pulse trains at least partially overlap each other.

The pulse trains can be configured in many different ways. Thus, the control device may control the stimulation device to vary the amplitudes of the pulses of the pulse trains, the duty cycle of the individual pulses of each pulse train, the width of each pulse of the pulse trains, the length of each pulse train, the repetition frequency of the pulses of the pulse trains, the repetition frequency of the pulse trains, the number of pulses of each pulse train, and/or the off time periods between the pulse trains. Several pulse trains of different configurations may be employed to achieve the desired effect.

In case the control device controls the stimulation device to vary the off time periods between pulse trains that stimulate the respective area of the wall portion, it is also possible to control each off time period between pulse trains to last long enough to restore substantially normal blood circulation in the area when the latter is not stimulated during the off time periods.

An electric stimulation device suitably comprises at least one, preferably a plurality of electrical elements, such as electrodes, for engaging and stimulating the wall portion with electric pulses. Optionally, the electrical elements may be placed in a fixed orientation relative to one another. The control device controls the electric stimulation device to electrically energize the electrical elements, one at a time, or groups of electrical elements at a time. Preferably, the control device controls the electric stimulation device to cyclically energize each element with electric pulses. Optionally, the control device may control the stimulation device to energize the electrical elements, such that the electrical elements are energized one at a time in sequence, or such that a number or groups of the electrical elements are energized at the same time. Also, groups of electrical elements may be sequentially energized, either randomly or in accordance with a predetermined pattern.

The electrical elements may form any pattern of electrical elements. Preferably, the electrical elements form an elongate pattern of electrical elements, wherein the electrical elements are applicable on the patient's wall of the organ, such that the elongate pattern of electrical elements extends lengthwise along the wall of the organ, and the elements abut the respective areas of the wall portion. The elongate pattern of electrical elements may include one or more rows of electrical elements extending lengthwise along the wall of the organ. Each row of electrical elements may form a straight, helical or zig-zag path of electrical elements, or any form of path. The control device may control the stimulation device to successively energize the electrical elements longitudinally along the elongate pattern of electrical elements in a direction opposite to, or in the same direction as that of, the flow in the patient's lumen.

In accordance with a preferred embodiment of the invention, the electrical elements form a plurality of groups of elements, wherein the groups form a series of groups extending along the patient's organ in the flow direction in the patient's lumen. The electrical elements of each group of electrical elements may form a path of elements extending at least in part around the patient's organ. In a first alternative, the electrical elements of each group of electrical elements may form more than two paths of elements extending on different sides of the patient's organ, preferably substantially transverse to the flow direction in the patient's lumen. The control device may control the stimulation device to energize the groups of electrical elements in the series of groups in random, or in accordance with a predetermined pattern. Alternatively, the control device may control the stimulation device to successively energize the groups of electrical elements in the series of groups in a direction opposite to, or in the same direction as that of, the flow in the patient's lumen, or in both said directions starting from a position substantially at the center of the constricted wall portion. For example, groups of energized electrical elements may form advancing waves of energized electrical elements, as described above; that is, the control device may control the stimulation device to energize the groups of electrical elements, such that energized electrical elements form two waves of energized electrical elements that simultaneously advance from the center of the constricted wall portion in two opposite directions towards both ends of the elongate pattern of electrical elements.

### Sensor

The control device may control the stimulation device to change the stimulation of the wall portion in response to a sensed physical parameter of the patient or functional parameter of the system. For example, the control device may control the stimulation device to increase the intensity of the stimulation of the wall portion in response to a sensed pressure increase in the oviduct, such that the flow in the oviduct remains stopped. Any sensor for sensing a physical parameter of the patient, such as a pressure in the patient's body that relates to the pressure in the oviduct may be provided, wherein the control device controls the stimulation device in response to signals from the sensor. Such a sensor may for example sense the pressure in the patient's abdomen, the pressure against the implanted constriction device or the pressure on the tissue wall of the bodily organ.

For example, a hormone level sensor may be applied where the present invention is used for controlling flow of the egg.

Sensor Controlled Restriction and/or Stimulation Device

As mentioned above, the system may comprise at least one implantable sensor, wherein the control device controls the constriction device and/or the stimulation device in response to signals from the sensor. Generally, the sensor directly or indirectly senses at least one physical parameter of the patient, or at least one functional parameter of the system, or at least one functional parameter of a medical implant in the patient.

The system is further preferably adapted to send feedback information from inside the body to the outside thereof to give feedback related to any functional parameter of the device or physical parameter of the patient.

The functional parameter of the device may be correlated to the transfer of energy for charging the internal energy source mentioned in other places.

The functional parameter of the device may be the energy balance, the balance between the energy received and the energy used including accumulated by the device and the energy balance could also include the balance between an energy reception rate and an energy using including accumulating rate.

Many different kinds of sensors for sensing physical parameters may be used.

The control device may comprise an implantable internal control unit that directly controls the constriction device and/or stimulation device in response to signals from the sensor. The control device may further comprise a wireless remote control adapted to set control parameters of the internal control unit from outside the patient without mechanically penetrating the patient. At least one of the control parameters, which is settable by the wireless remote control, is the physical or functional parameter. Suitably, the internal control unit includes the above mentioned clock mechanism, wherein the wireless remote control also is adapted to set the clock mechanism.

Alternatively, the control device may comprise an external control unit outside the patient's body for controlling the constriction device and/or stimulation device in response to signals from the sensor.

### Adjustable Constriction Device

In several alternative embodiments of the invention, the constriction device is adjustable. In these embodiments, there is an operation device for operating the adjustable constriction device to change the constriction of the patient's tissue wall portion, and the constriction and stimulation devices form a constriction/stimulation unit. Preferably, the constriction and stimulation devices of the constriction/stimulation unit are integrated in a single piece suitable for implantation. The constriction device of the unit comprises contact surfaces dimensioned to contact a length of a tissue wall portion of a patient's organ, and the stimulation device of the unit comprises a plurality of stimulation elements provided on and distributed along the contact surfaces. When the control device controls the stimulation device to stimulate the wall portion, the stimulation elements stimulate different areas of the wall portion along the length of the wall portion. The stimulation elements preferably comprise electric elements, as described above, for stimulating the wall portion with electric pulses. However, in most applications of the present invention, other kinds of stimulations, such as thermal stimulation, could be suitable to employ.

The operation device operates the adjustable constriction device of the constriction/stimulation unit in a manner that depends on the design of the constriction device, as will be explained by the following examples of embodiments. 1) The constriction device comprises at least two elongated clamping elements having the contact surfaces and extending along the wall portion on different sides of the organ, and the operation device operates the clamping elements to clamp the wall portion between the clamping elements to constrict the wall portion of the organ.
2) The constriction device comprises one elongate clamping element having the contact surfaces and extending along the wall portion on one side of the organ, and the operation device operates the clamping element to clamp the wall portion between the clamping element and the bone or tissue of the patient to constrict the wall portion.
3) The constriction device comprises at least two engagement elements having the contact surfaces and positioned on different sides of the organ, and the operation device rotates the engagement elements, such that the engagement elements engage and constrict the wall portion of the organ.
4) The constriction device comprises at least two articulated clamping elements having the contact surfaces and positioned on different sides of the organ, and the operation device moves the clamping elements towards each other to clamp the wall portion of the organ between the clamping elements, to constrict the wall portion.
5) The constriction device comprises at least two separate clamping elements having the contact surfaces, at least one of the clamping elements being pivoted, such that it may turn in a plane in which the loop of the constriction member extends, and the operation device turns the pivoted clamping element to change the size of the constriction opening.
6) The constriction device comprises at least one elongated constriction member having the contact surfaces, and forming means for forming the constriction member into at least a substantially closed loop around the organ, wherein the loop defines a constriction opening. The operation device operates the constriction member in the loop to change the size of the constriction opening.
   6a) The elongated constriction member comprises a belt having the contact surfaces, and the operation device operates the belt to change the longitudinal extension of the belt in the loop to change the size of the constriction opening. The forming means may form the constriction member or belt into a loop having at least one predetermined size.
   6b) The elongated constriction member is operable to change the size of the constriction opening, such that the outer circumferential confinement surface of the constriction device is changed, or, alternatively, is unchanged.
   6c) The elongated constriction member is elastic and varies in thickness as seen in a cross-section there through, and is operable to turn around the longitudinal extension of the constriction member.
   6d) The elongated constriction member comprises two substantially or partly semi-circular frame elements having the contact surfaces and hinged together, such that the semi-circular elements are swingable relative to each other from a fully open state in which they substantially or partly form a circle to a fully folded state in which they substantially form a semi-circle.
7) The constriction device is adapted to bend the wall portion of the organ to constrict the latter.

In the above noted embodiments (1) to (7), it is important that the constriction device is designed to constrict said length of the tissue wall portion of the patient's organ. For this purpose, the constriction device may include two or more of the described constriction elements/members to be applied in a row along said length of the wall portion, wherein said row extends in the direction of flow in the oviduct of the organ. Preferably, such constriction elements/members are non-inflatable and mechanically operable or adjustable.

In the above noted embodiments (1) to (7), the operation device may either mechanically or hydraulically adjust the constriction device of the constriction/stimulation unit. Also, the operation device may comprise an electrically powered operation device for operating the constriction device. For many applications of the present invention, the operation device suitably operates the constriction device, such that the through-flow area of the oviduct assumes a size in the constricted state that enables the stimulation device to contract the wall portion such that the flow in the oviduct is stopped.

### Mechanical Operation

Where the operation device mechanically operates the constriction device of the constriction/stimulation unit, it may be non-inflatable. Furthermore, the operation device may comprise a servo system, which may include a gearbox. The term “servo system” encompasses the normal definition of a servo mechanism, i.e., an automatic device that controls large amounts of power by means of very small amounts of power, but may alternatively or additionally encompass the definition of a mechanism that transfers a weak force acting on a moving element having a long stroke into a strong force acting on another moving element having a short stroke. Preferably, the operation device operates the constriction device in a non-magnetic and/or non-manual manner. A motor may be operatively connected to the operation device. The operation device may be operable to perform at least one reversible function and the motor may be capable of reversing the function.

### Hydraulic Operation

Where the operation device hydraulically operates the constriction device of the constriction/stimulation unit, it includes hydraulic means for adjusting the constriction device.

In an embodiment of the invention, the hydraulic means comprises a reservoir and an expandable/contractible cavity in the constriction device, wherein the operation device distributes hydraulic fluid from the reservoir to expand the cavity, and distributes hydraulic fluid from the cavity to the reservoir to contract the cavity. The cavity may be defined by a balloon of the constriction device that abuts the tissue wall portion of the patient's organ, so that the patient's wall portion is constricted upon expansion of the cavity and released upon contraction of the cavity.

Alternatively, the cavity may be defined by a bellows that displaces a relatively large contraction element of the constriction device, for example a large balloon that abuts the wall portion, so that the patient's wall portion is constricted upon contraction of the bellows and released upon expansion of the bellows. Thus, a relatively small addition of hydraulic fluid to the bellows causes a relatively large increase in the constriction of the wall portion. Such a bellows may also be replaced by a suitably designed piston/cylinder mechanism.

Where the hydraulic means comprises a cavity in the constriction device, the system can be designed in accordance with the options listed below.
1) The reservoir comprises first and second wall portions, and the operation device displaces the first and second wall portions relative to each other to change the volume of the reservoir, such that fluid is distributed from the reservoir to the cavity, or from the cavity to the reservoir.
   1a) The first and second wall portions of the reservoir are displaceable relative to each other by at least one of a magnetic device, a hydraulic device or an electric control device.
2) The operation device comprises a pump for pumping fluid between the reservoir and the cavity.
   2a) The pump comprises a first activation member for activating the pump to pump fluid from the reservoir to the cavity and a second activation member for activating the pump to pump fluid from the cavity to the reservoir.
   2a1) The first and second activation members are operable by manual manipulation thereof.
   2a2) At least one of the activation members operates when subjected to an external predetermined pressure.
   2a3) At least one of the first and second activating members is operable by magnetic means, hydraulic means, or electric control means.
   2b) The system comprises a fluid conduit between the pump and the cavity, wherein the reservoir forms part of the conduit. The conduit and pump are devoid of any non-return valve. The reservoir forms a fluid chamber with a variable volume, and the pump distributes fluid from the chamber to the cavity by a reduction in the volume of the chamber and withdraws fluid from the cavity by an expansion of the volume of the chamber. The system further comprises a motor for driving the pump, wherein the pump comprises a movable wall of the reservoir for changing the volume of the chamber.

In all of the above noted embodiments 1 to 2b where the hydraulic means comprises an expandable cavity in the constriction device, the cavity can be exchanged by a cylinder/piston mechanism for adjusting the constriction device. In this case, the operation device distributes hydraulic fluid between the reservoir and the cylinder/piston mechanism to adjust the constriction device.

In a special embodiment of the invention, the operation device comprises a reverse servo operatively connected to the hydraulic means. The term “reverse servo” is to be understood as a mechanism that transfers a strong force acting on a moving element having a short stroke into a weak force acting on another moving element having a long stroke; i.e., the reverse function of a normal servo mechanism. Thus, minor changes in the amount of fluid in a smaller reservoir could be transferred by the reverse servo into major changes in the amount of fluid in a larger reservoir. The reverse servo is particularly suited for manual operation thereof.

Preferably, the reverse servo comprises an expandable servo reservoir containing servo fluid and a fluid supply reservoir hydraulically connected to the servo reservoir to form a closed conduit system for the servo fluid. The expandable servo reservoir has first and second wall portions, which are displaceable relative to each other in response to a change in the volume of the expandable servo reservoir.

In accordance with a first alternative, the first and second wall portions of the servo reservoir are operatively connected to the hydraulic means. The reverse servo distributes fluid between the fluid supply reservoir and the expandable servo reservoir to change the volume of the servo reservoir, whereby the hydraulic means is operated to adjust the constriction device.

In accordance with a second alternative, there is provided an implantable main reservoir containing a predetermined amount of hydraulic fluid, wherein the reverse servo is operable to distribute hydraulic fluid between the main reservoir and the hydraulic means to adjust the constriction device. More specifically, the main reservoir is provided with first and second wall portions operatively connected to the first and second wall portions of the expandable servo reservoir, such that the volume of the main reservoir is changed when the volume of the expandable servo reservoir is changed. Thus, when the reverse servo distributes servo fluid between the fluid supply reservoir and the expandable servo reservoir to change the volume of the main reservoir, hydraulic fluid is distributed from the main reservoir to the hydraulic means, or from the hydraulic means to the main reservoir. Advantageously, the servo and main reservoirs are dimensioned, such that when the volume of the servo reservoir is changed by a relatively small amount of servo fluid, the volume of the main reservoir is changed by a relatively large amount of hydraulic fluid.

In both of the above-described alternatives, the fluid supply reservoir may have first and second wall portions, which are displaceable relative to each other to change the volume of the fluid supply reservoir to distribute servo fluid between the fluid supply reservoir and the expandable servo reservoir. The first and second wall portions of the fluid supply reservoir may be displaceable relative to each other by manual manipulation, a magnetic device, a hydraulic device, or an electric control device to change the volume of the fluid supply reservoir to distribute servo fluid between the fluid supply reservoir and the expandable servo reservoir.

In all of the above noted embodiments 1 to 2b where the hydraulic means comprises an expandable cavity in the constriction device, or in embodiments where the hydraulic means comprises a hydraulically operable mechanical construction, the operation device may include the reverse servo described above. In a further embodiment of the invention, the hydraulic means include first and second hydraulically interconnected expandable/contractible reservoirs. The first reservoir is operatively connected to the constriction device, such that the constriction device changes the constriction of the patient's wall portion upon expansion or contraction of the first reservoir. By changing the volume of the second reservoir hydraulic fluid is distributed between the two reservoirs, so that the first reservoir is either expanded or contracted. This embodiment requires no non-return valve in the fluid communication conduits between the two reservoirs, which is beneficial to long-term operation of the hydraulic means.

Alternatively, the hydraulic means may include first and second hydraulically interconnected piston/cylinder mechanisms instead of the first and second reservoirs described above. The first piston/cylinder mechanism is operatively connected to the constriction device, such that the constriction device changes the constriction of the patient's wall portion upon operation of the first piston/cylinder mechanism. By operating the second piston/cylinder mechanism hydraulic fluid is distributed between the two piston/cylinder mechanisms, so that the first piston/cylinder mechanism adjusts the constriction device.

Where the constriction device does not include an expandable/contractible cavity, the constriction device may comprise at least two elongated clamping elements having the above-mentioned contact surfaces and extending along the wall portion on different sides of the organ. The hydraulic means, which may include the reverse servo described above, hydraulically moves the elongated clamping elements towards the wall portion to constrict the wall portion. For example, the constriction device may have hydraulic chambers in which the clamping elements slide back and forth, and the hydraulic means may also include a pump and an implantable reservoir containing hydraulic fluid. The pump distributes hydraulic fluid from the reservoir to the chambers to move the clamping elements against the wall portion, and distributes hydraulic fluid from the chambers to the reservoir to move the clamping elements away from the wall portion.

More than One Restriction Area

The restriction device is independent of which type or combination of types preferable. It comprises more than one restriction area, thereby being adapted to change the restriction area over time. This will prevent any damage to the oviduct still keeping the oviduct closed avoiding any egg to pass down to the uterus thus avoiding pregnancy.

I one embodiment the system comprises a hydraulic restriction device with two or more restriction areas connected individually to two or more reservoirs with hydraulic fluid, said reservoirs adapted to be regulated to move fluid from said reservoirs individually to each of the connected restriction areas. It is possible to use only one reservoir if valves instead control on which restriction the hydraulic fluid is acting.

The hydraulic restriction areas are adapted to be restricted for a predetermined time period, preferably with some overlap in time and adapted to first restrict the restriction area closest to the ovary and then changing restriction area towards the uterus. This way the restriction may all the time be kept but without risking to have any egg to pass when changing restriction area both because of the overlap in restriction and also because restriction is started first closest to the ovary where the egg is released.

The hydraulic restriction areas may be adapted to be regulated by manual manipulation thereof.

The system may also be adapted to that the change of the restriction area cause a peristaltic wave like restriction wave in the direction towards the ovary to prevent the egg being transported down to the uterus. This peristaltic wave may be caused by any of the different types of restriction devices or combinations described earlier. This wave may prevent the egg being transported down to the uterus also without fully restricting the oviduct. This would then allow the sperm to reach the egg during the accumulation period of a few days, thus increasing likelihood of pregnancy even further.

The restriction device may also be adapted to affect a transport of the at least one egg to the uterus upon release of the oviduct. This may be with a peristaltic wave like restriction wave in the opposite direction towards the uterus.

A system of preventing or increasing likelihood of pregnancy may be adapted to restrict a first part or area of an oviduct of the patient to provide a restriction to accumulate at least one egg released from the ovary in the oviduct for a predetermined period of time, and adapted to restrict a second part of the oviduct and thereafter release the restriction of the first part and also later release the restriction of the second part, thereby allow transport of the egg down to the uterus or the system may further be adapted to also restrict a third part of the oviduct and release the restriction of the second part also it may further be adapted to release the restriction of the third part thereby allow transport of the egg down to the uterus or the system may be adapted to restrict a fourth part of the oviduct and release the restriction of the third part. Finally it may be further adapted to release the restriction of the fourth part, thereby allow transport of the egg down to the uterus.

The number of restriction areas has no limit except for practical size issues and the restriction time period is preferably divided between the restriction areas. Either the restriction area is moved from the ovary and further down towards the uterus step by step thus avoiding any interference with any accumulated egg being involved in any restriction area if a certain overlap in time is fulfilled between the consecutive restriction areas in use or the restriction areas are moved both up- and down-stream then preferable using a movement device to move the egg in the oviduct to avoid any egg being squeezed in any restriction area. This may occur when the restriction areas are moved towards the ovary or having the peristaltic like wave moving towards the ovary keeping the egg accumulated in the oviduct without fully restricting the same.

Preferably a hydraulic restriction device only partly restricting the oviduct may be used in combination with a stimulation device to completely close or cause the peristaltic wave in the oviduct. With the stimulation device it is then possible to move the restriction area. The hydraulic device may for example have the movement function to cause the movements necessary.

Any combination is possible of mechanical, hydraulic or stimulation device or separate use of the same.

In summary preferably more than two restricting areas are used and varying the restricting area while at least one restricting area is closed when the device being in restriction mode.

If the device is adapted to restrict the first part of the oviduct closest to the ovary it will allow the second restriction being restricted without interfering with any accumulated egg. There is no limit how many areas could be restricted except practical and size issues.

Moving in restriction from the ovary and down towards the uterus is then the preferred way of doing it, for example three restrictions could share a time period of three days with one day each to not risking to in any way damage the oviduct,

A system adapted to restrict the restriction areas in consecutive order starting with the restriction area, part of the oviduct, closest to the ovary and thereafter restrict any new area one step closer to the uterus and further adapted to overlap in time the restriction of more than one restriction area will allow to restrict without interfering with any accumulated egg.

A method of avoiding pregnancy of a female mammal or human patient comprises the following steps:
restricting an oviduct of the patient postoperatively to provide a restriction to accumulate at least one egg released from the ovary in the oviduct for a predetermined period of time,
releasing the restriction to admit any egg in the oviduct a transport to the uterus, and
controlling the restricting and releasing procedures from outside the patient's body.

In this and other methods the predetermined period of time is adapted to avoid pregnancy and may be between 2 and 30 days or more than 30 days. It may also be between 0 and 2 days or 8-12 hours.

A method for placing and controlling two implanted restriction devices avoiding or promoting pregnancy in a human or mammal patient comprises the steps of:
inserting a needle or tube-like instrument into the abdomen of the patient's body,
using the needle or tube-like instrument to fill the abdomen with gas thereby expanding the abdominal cavity,
placing at least two laparoscopic trocars in the patient's body,
inserting a camera through one of the trocars into the abdomen,
inserting at least one dissecting tool through a trocar and dissecting an area of at least one portion of the two oviducts of the patient,
placing two implanted restriction devices, on each of the two oviducts
adjusting the restriction devices after the operation at a time convenient to not get pregnant or to get pregnant thus,
controlling the adjustment from outside the patient's body and
post-operatively restricting the two oviducts to avoid getting pregnant alternatively post-operatively restricting and releasing the two oviducts to increase the likelihood of getting pregnant.

Both methods above could when restricting the oviduct comprising the following steps:
restricting a first part or area of an oviduct of the patient to provide a restriction to accumulate at least one egg released from the ovary in the oviduct for a predetermined period of time,
restricting a second part of the oviduct,
releasing the restriction of the first part, and
allowing the oviduct shorter restriction periods at each restriction area.

The method may also comprise the steps of:
releasing the restriction of the second part, and
allowing transport of the egg down to the uterus.

The method may also comprise the steps of:
restricting a third part of the oviduct,
releasing the restriction of the second part, and
allowing the oviduct shorter restriction periods at each restriction area.

The method may also comprise the steps of:
releasing the restriction of the third part, and
allowing transport of the egg down to the uterus.

The method may also comprise the steps of:
restricting a fourth part of the oviduct,
releasing the restriction of the third part, and
allowing the oviduct shorter restriction periods at each restriction area.

The method may also comprise the steps of:
releasing the restriction of the fourth part, and
allowing transport of the egg down to the uterus.

In both these methods, normally if the restricted first part of the oviduct is closer to the ovary this is,
allowing the second restriction being restricted without interfering with any accumulated egg.

Normally these methods comprise
having more than two restricting areas and
varying the restricting area while
keeping at least one restricting area closed when restriction is desired.

In a preferred embodiment, the restriction areas are restricted in consecutive order starting with the restriction area, part of the oviduct, closest to the ovary thereafter,
restricting any new area one step closer to the uterus,
overlapping in time the restriction of more than one restriction area thereby
restricting without interfering with any accumulated egg

A method may of course only include, independently of order, varying the restriction area postoperatively to allow the oviduct to recover or to avoid any damage from the restriction while keeping the oviduct restricted.

Another method of preventing or promoting pregnancy of a female patient comprises the following steps:
preventing the transport of an egg in an oviduct to the uterus of the human or mammal patient,
accumulating at least one egg released from the ovary in the oviduct for a predetermined period of time, by
casing a peristaltic like restriction wave movement of a part of the oviduct(s) wall preventing the egg being transported down to the uterus while restricting the oviduct at all times,
preventing or allowing the sperm to reach the egg during the time the egg is accumulated, and
releasing the egg post-operatively controlled from the outside the human body to admit the at least on egg in the oviduct to allow a transport of the at least one egg to the uterus.

A method for placing the device above and controlling the implanted device avoiding pregnancy in a human or mammal patient comprises the steps of:
inserting a needle or tube like instrument into the abdomen of the patient's body,
using the needle or tube like instrument to fill the abdomen with gas thereby expanding the abdominal cavity,
placing at least two laparoscopic trocars in the patient's body,
inserting a camera through one of the trocars into the abdomen,
inserting at least one dissecting tool through a trocar and dissecting an area of at least one portion of the two oviducts of the patient,
placing two parts of the implanted device, one each on the two oviducts
finishing the operation and withdrawing the instruments after eventual suturing and thereafter postoperatively:
adjusting the device after the operation at a time relevant to avoid or promote getting pregnant,
controlling the adjustment from outside the patient's body and thereby
preventing flow of any egg to reach the uterus in the two oviducts for a predetermined period of time to avoid getting pregnant and thereby
accumulating any egg released from the ovary in the oviduct(s) by
casing a peristaltic like wave movement of a part of the oviduct(s) wall preventing the egg being transported down to the uterus restricting the oviduct at all times,
preventing sperms from reaching the egg during the time the egg is accumulated, and
releasing any egg in the oviduct from outside the body to allow the egg to in a normal way be transported down to the uterus when risk for pregnancy is low.

A method for placing the device above and controlling the implanted device promoting pregnancy in a human or mammal patient, the method comprising the steps of:
inserting a tube like needle into the abdomen of the patient's body,
using the tube like needle like to fill the abdomen with gas thereby expanding the abdominal cavity,
placing at least two laparoscopic trocars in the patient's body,
inserting a camera through one of the trocars into the abdomen,
inserting at least one dissecting tool through a trocar and dissecting an area of at least one portion of the two oviducts of the patient,
placing two parts of the implanted device, one each on the two oviducts—finishing the operation and withdrawing the instruments after eventual suturing and thereafter postoperatively:
adjusting the device after the operation at a time relevant to get pregnant,
controlling the adjustment from outside the patient's body and thereby
casing a peristaltic like wave movement of a part of the oviduct(s) wall towards the ovary preventing the egg being transported down to the uterus without fully restricting the oviduct, thereby
preventing flow of any egg to reach the uterus in the two oviducts for a predetermined period of time and thereby
accumulating any egg released from the ovary in the oviduct(s) by
allowing the sperm to reach the egg during the time the egg is accumulated
releasing any egg in the oviduct from outside the body to allow the egg to in a normal way be transported down to the uterus.

### Restriction Embodiments

The restriction device may comprise a mechanical restriction device or a hydraulic restriction device or a stimulation device or a stimulation device in combination with a mechanical or hydraulic restriction device or any other combination.

The method may include a hydraulic restriction device comprising a reservoir, for moving gas or fluid to or from said restriction device and wherein said reservoir is placed subcutaneously for
i. being reached by the patients hand for
ii. moving fluid manually to or from said restriction device.

Preferable two or more reservoirs could be used for restricting different areas, starting with the area closest to the ovary.

### Peristaltic Like Wave Oviduct Wall Movements

The restriction device may be adapted to only partly restrict the oviduct and to create peristaltic wave like movements of a part of the oviduct(s) wall to prevent the transport of an egg in an oviduct to the uterus of the human or mammal patient, and therefore accumulate at least one egg released from the ovary in the oviduct for a predetermined period of time, and wherein the spenm is able to reach the egg during the time the egg is accumulated because the oviduct is only partly restricted, and the restriction device further adapted to release the egg, controlled from outside the body, to allow the at least on egg in the oviduct a transport to the uterus.

The device is then preferable adapted to comprise at least one restricted area adapted to at least partly restrict and change the restriction area over time most likely adapted to comprise two or more restricted areas adapted to at least partly restrict different portions of the oviduct and change the restriction area over time.

The change of the restriction area may be adapted to cause a peristaltic wave like wave in the direction towards the ovary to prevent the egg being transported down to the uterus.

The restriction device may comprise a hydraulic device or a mechanical device or a stimulation device adapted to cause said peristaltic wave like restriction wave.

The device is adapted to allow a transport of the at least one egg to the uterus upon release of the oviduct and may even cause a another peristaltic wave like restriction wave in the opposite direction towards the uterus.

The device is adapted to be adjusted which could be done by manual manipulation or electrical or magnetic power.

The restriction areas is only partly restricted to allow the spern to reach the egg and the device adapted to prevent the egg to temporary reach the uterus with the peristaltic wave. to accumulate the egg temporary in the oviduct to allow increased likelihood for pregnancy to prolong the time period the egg being reachable by the sperm.

When the restriction areas is completely restricted to prevent the egg to temporary reach the uterus, the timing of the release of the egg is crucial after having accumulated the egg temporary in the oviduct the whole procedure to allow increased likelihood for pregnancy when released.

The restriction device is adapted to and intended to repeatedly every month repeat the post-operational and non-invasive regulation of the device actively reversible to prevent transport of an egg from the ovary to the uterus in the oviduct of the patient and reverse the function by allowing the normal transport of the egg from the ovary to the uterus until pregnancy has been achieved.

The device may comprise a mechanical device or a hydraulic device or a stimulation device adapted to cause said peristaltic like wave towards the ovary.

Restriction embodiments The restriction device may comprises a mechanical restriction device or a hydraulic restriction device or a stimulation device or a stimulation device in combination with a mechanical or hydraulic restriction device or any other combination.

The method may include a hydraulic restriction device comprising a reservoir, for moving gas or fluid to or from said restriction device and wherein said reservoir is placed subcutaneously for
i. being reached by the patients hand for
ii. moving fluid manually to or from said restriction device

Preferable two or more reservoirs could be used for restricting different areas, starting with the area closest to the ovary.

### Method Movement Device

The movement device is adapted to move the egg out from a varied upcoming new restricted area before a new area is restricted useful if the restriction should be kept for a longer period of time and the restriction area therefore needs to be moved also in the direction towards the ovary in which case the egg could slip through if being squeezed by one restriction area and later released.

The movement device may be the same device as the restriction device, adapted to work differently when restricting or causing movements of the egg or it may be a different device as the restriction device adapted to work differently when restricting or causing movements of the egg. The movement device may cause vibration or wave like movements in the oviduct wall thereby causing movements of the egg.

Therefore another method of preventing pregnancy of a female human or mammal patient may be provided, comprising the following steps:
restricting a first part of an oviduct of the patient to provide a restriction to accumulate at least one egg released from the ovary in the oviduct for a predetermined period of time, and
moving the accumulated egg by said movement device away in the oviduct towards the ovary from the restriction area,
allowing a second part of the oviduct closer to the ovary being restricted without interfering with any accumulated egg,
releasing the restriction of the first part,
repeating the restriction of the first part,
releasing the restriction of the second part,
allowing the oviduct to recover between restriction intervals.

These steps are preferably followed by;
repeating moving any accumulated egg by said movement device away in the oviduct towards the ovary from the restricted first area, repeating
allowing a second part of the oviduct closer to the ovary being restricted without interfering with any accumulated egg, further repeating,
releasing the restriction of the first part,
repeating the complete procedure allowing the oviduct to recover between restriction intervals.

Preferably also the restriction areas are adapted to be varied between three or more areas while always keeping the oviduct closed.

Said movement device may comprise a vibrating device, for causing a vibration of at least a part of the wall of said oviduct causing movement of any accumulated egg, said movement being repeated.

Said movement device may comprise a mechanical device or a hydraulic device or a stimulation device or a combined device.

### Design of Control Device

The control device suitably controls the constriction and/or stimulation unit from outside the patient's body. Preferably, the control device is operable by the patient. For example, the control device may comprise a manually operable switch for switching on and off the constriction/stimulation unit, wherein the switch is adapted for subcutaneous implantation in the patient to be manually or magnetically operated from outside the patient's body. Alternatively, the control device may comprise an external control unit in the form of a hand-held wireless remote control, which is conveniently operable by the patient to switch on and off the constriction/stimulation unit. The wireless remote control may also be designed for application on the patient's body like a wristwatch. Such a wristwatch type of remote control may emit a control signal that follows the patient's body to implanted signal responsive means of the system.

Where the control device wirelessly controls the constriction/stimulation unit from outside the patient's body, the wireless control function is preferably performed in a non-magnetic manner, i.e., the control device controls the constriction device of the constriction/stimulation unit in a non-magnetic manner. The patient may use the remote control to control the constriction/stimulation unit to adjust the stimulation intensity and/or adjust the constriction of the wall portion. The wireless remote control may comprise at least one external signal transmitter or transceiver and at least one internal signal receiver or transceiver implantable in the patient.

The wireless remote control preferably transmits at least one wireless control signal for controlling the constriction/stimulation unit. The control signal may comprise a frequency, amplitude, phase modulated signal or a combination thereof, and may be an analogue or a digital signal, or a combination of an analogue and digital signal. The remote control may transmit an electromagnetic carrier wave signal for carrying the digital or analogue control signal. Also the carrier signal may comprise digital, analogue or a combination of digital and analogue signals.

Any of the above control signals may comprise wave signals, for example a sound wave signal, an ultrasound wave signal, an electromagnetic wave signal, an infrared light signal, a visible light signal, an ultra violet light signal, a laser light signal, a microwave signal, a radio wave signal, an x-ray radiation signal or a gamma radiation signal. Alternatively, the control signal may comprise an electric or magnetic field, or a combined electric and magnetic field.

As mentioned above, the control signal may follow the patient's body to implanted signal responsive means of the system.

The control device may include a programmable internal control unit, such as a microprocessor, implantable in the patient for controlling the constriction/stimulation unit. The control device may further include an external control unit intended to be outside the patient's body, wherein the internal control unit is programmable by the external control unit. For example, the internal control unit may be programmable for controlling the constriction/stimulation unit over time, suitably in accordance with an activity schedule program. The system may comprise an external data communicator and an implantable internal data communicator communicating with the external data communicator, wherein the internal communicator feeds data related to the constriction/stimulation unit back to the external data communicator or the external data communicator feeds data to the internal data communicator.

### Source of Energy

The present invention also presents a solution for supplying energy for use in connection with the operation of the constriction/stimulation unit. Thus, in a broad sense, the present invention provides an system for controlling a flow of egg in a oviduct formed by a tissue wall of a patient's organ, wherein the system comprises an implantable constriction device for gently constricting a portion of the tissue wall to influence the flow in the oviduct, a stimulation device for intermittently and individually stimulating different areas of the wall portion, as the constriction device constricts the wall portion, to cause contraction of the wall portion to further influence the flow in the oviduct, wherein the constriction and stimulation devices form an operable constriction/stimulation unit, a source of energy, and a control device operable from outside the patient's body to control the source of energy to release energy for use in connection with the operation of the constriction/stimulation unit. In a simple form of the invention, the source of energy, such as a battery or accumulator, is implantable in the patient's body.

### Transmission of Wireless Energy

In a more sophisticated form of the invention, which is preferable, the source of energy is external to the patient's body and the control device controls the external source of energy to release wireless energy. In this sophisticated form of the invention, the system comprises an energy-transmission device that transmits the released wireless energy from outside the patient's body to inside the patient's body. Among many things the wireless energy may comprise electromagnetic energy, an electric field, an electromagnetic field or a magnetic field, or a combination thereof, or electromagnetic waves. The energy-transmission device may transmit wireless energy for direct use in connection with the operation of the constriction/stimulation unit, as the wireless energy is being transmitted. For example, where an electric motor or pump operates the constriction device, wireless energy in the form of a magnetic or an electromagnetic field may be used for direct power of the motor or pump.

Thus, the motor or pump is running directly during transmission of the wireless energy. This may be achieved in two different ways: a) using a transforming device implanted in the patient to transform the wireless energy into energy of a different form, preferably electric energy, and powering the motor or pump with the transformed energy, or b) using the wirelessly transmitted energy to directly power the motor or pump. Preferably wireless energy in the form of an electromagnetic or magnetic field is used to directly influence specific components of the motor or pump to create kinetic energy for driving the motor or pump. Such components may include coils integrated in the motor or pump, or materials influenced by magnetic fields, or permanent magnets, wherein the magnetic or electromagnetic field influences the coils to generate a current for driving the motor or pump, or influences the material or permanent magnets to create kinetic energy for driving the motor or pump.

Preferably, the energy-transmission device transmits energy by at least one wireless signal, suitably a wave signal. The wave signal may comprise an electromagnetic wave signal including one of an infrared light signal, a visible light signal, an ultra violet light signal, a laser signal, a microwave signal, a radio wave signal, an x-ray radiation signal, and a gamma radiation signal. Alternatively, the wave signal may comprise a sound or ultrasound wave signal. The wireless signal may be a digital or analogue signal, or a combination of a digital and analogue signal.

### Transforming Wireless Energy

In accordance with a particular embodiment of the invention, an implantable energy-transforming device is provided for transforming wireless energy of a first form transmitted by the energy-transmission device into energy of a second form, which typically is different from the energy of the first form. The constriction/stimulation unit is operable in response to the energy of the second form. For example, the wireless energy of the first form may comprise sound waves, whereas the energy of the second form may comprise electric energy. In this case, the energy-transforming device may include a piezo-electric element for transforming the sound waves into electric energy. Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transforming device may function differently from or similar to the energy-transmission device. In a special embodiment, the energy-transforming device comprises at least one element, such as at least one semiconductor, having a positive region and a negative region, when exposed to the energy of the first form transmitted by the energy-transmission device, wherein the element is capable of creating an energy field between the positive and negative regions, and the energy field produces the energy of the second form. More specifically, the element may comprise an electrical junction element, which is capable of inducing an electric field between the positive and negative regions when exposed to the energy of the first form transmitted by the energy-transmission device, whereby the energy of the second form comprises electric energy.

The energy-transforming device may transform the energy of the first form directly or indirectly into the energy of the second form. An implantable motor or pump for operating the constriction device of the constriction/stimulation unit may be provided, wherein the motor or pump is powered by the energy of the second form. The constriction device may be operable to perform at least one reversible function and the motor may be capable of reversing the function. For example, the control device may shift polarity of the energy of the second form to reverse the motor.

The energy-transforming device may directly power the motor or pump with the transformed energy, as the energy of the second form is being transformed from the energy of the first form. Preferably, the energy-transforming device directly operates the constriction/stimulation unit with the energy of the second form in a non-magnetic, non-thermal or non-mechanical manner.

Normally, the constriction/stimulation unit comprises electric components that are energized with electrical energy. Other implantable electric components of the system may be at least one voltage level guard or at least one constant current guard. Therefore, the energy-transforming device may transform the energy of the first form into a direct current or pulsating direct current, or a combination of a direct current and pulsating direct current. Alternatively, the energy-transforming device may transform the energy of the first form into an alternating current or a combination of a direct and alternating current.

The system may comprise an internal source of energy implantable in the patient for supplying energy for the operation of the constriction/stimulation unit. The system may further comprise an implantable switch operable to switch from an “off” mode, in which the internal source of energy is not in use, to an “on” mode, in which the internal source of energy supplies energy for the operation of the constriction/stimulation unit, and/or for energizing implanted electronic components of the system. The switch may be operable by the energy of the first form transmitted by the energy-transmission device or by the energy of the second form supplied by the energy-transforming device. The described switch arrangement reduces power consumption of the system between operations.

The internal source of energy may store the energy of the second form supplied by the energy-transforming device. In this case, the internal source of energy suitably comprises an accumulator, such as at least one capacitor or at least one rechargeable battery, or a combination of at least one capacitor and at least one rechargeable battery. Where the internal source of energy is a rechargeable battery it may be charged only at times convenient for the patient, for example when the patient is sleeping.

Alternatively, the internal source of energy may supply energy for the operation of the constriction/stimulation unit but not be used for storing the energy of the second form. In this alternative, the internal source of energy may be a battery and the switch described above may or may not be provided.

Suitably, the system comprises an implantable stabilizer for stabilizing the energy of the second form. Where the energy of the second form is electric energy the stabilizer suitably comprises at least one capacitor.

The energy-transforming device may be designed for implantation subcutaneously in the abdomen, thorax or cephalic region of the patient. Alternatively, it may be designed for implantation in an orifice of the patient's body and under the mucosa or intramuscularly outside the mucosa of the orifice.

Although the constriction/stimulation unit in the embodiments described above is designed as a single piece, which is most practical for implantation, it should be noted that as an alternative the constriction device and stimulation device could be designed as separate pieces. Any one of the constriction and stimulation units described above may alternatively be replaced by two or more separate constriction/stimulation elements, which are controlled independently of one another.

Where the a system is used for controlling the flow of eggs into the uterus of a female, the system comprises an implantable constriction device for constricting each one of the female's uterine tubes to restrict the passageway thereof, and a control device for controlling said constriction device to constrict the uterine tube such that an egg appearing in the passageway of the uterine tube is prevented from entering the uterine cavity, and to release the uterine tube such that an egg existing in the passageway of the uterine tube is allowed to enter the uterine cavity. The constriction device may gently constrict at least one portion of the tissue wall of the uterine tube to restrict the passageway thereof, and an implantable stimulation device may be provided for stimulating the tissue wall portion, wherein the control device controls said stimulation device to stimulate the tissue wall portion, as said constriction device constricts the tissue wall portion, to cause contraction of the tissue wall portion to further restrict the passageway of the uterine tube.

Alternatively, the egg flow control system comprises an implantable constriction device for gently constricting at least one portion of the tissue wall of each one of the female's uterine tubes to restrict the passageway thereof, a stimulation device for stimulating the tissue wall portion of the uterine tube, and a control device for controlling said stimulation device to stimulate the tissue wall portion, as said constriction device constricts the tissue wall portion, to cause contraction of the tissue wall portion to further restrict the passageway of the uterine tube to prevent an egg existing in the uterine tube from entering the uterine cavity.

Alternatively, the egg flow control system comprises an implantable stimulation device for stimulating a portion of the tissue wall of each one of the female's uterine tubes, and a control device for controlling said stimulation device to stimulate the tissue wall portion of the uterine tube to cause contraction of the tissue wall portion, such that the passageway of the uterine tube is restricted to prevent an egg appearing in the uterine tube from entering the uterine cavity, and to cease stimulating the tissue wall portion of the uterine tube to allow an egg existing in the passageway of the uterine tube to enter the uterine cavity.

The present invention also provides a method for using an system as described above to control a flow of egg in a oviduct formed by a tissue wall of a patient's organ, the method comprising:
providing a wireless remote control adapted to control the constriction device and/or stimulation device from outside the patient's body, and
operating the wireless remote control by the patient, when the patient wants to influence the flow of egg in the oviduct.

The present invention also provides a method for controlling a flow of egg in a oviduct formed by a tissue wall of a patient's organ, the method comprising:
a) gently constricting at least one portion of the tissue wall to influence the flow in the oviduct, and
b) stimulating the constricted wall portion to cause contraction of the wall portion to further influence the flow in the oviduct.

### Q: Controlling Movement of Sperms

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for controlling the movement of sperms in the uterine tubes of a female patient, examples of such devices for controlling the movement of sperms in the uterine tubes of a female patient will now be described.

The object of the present invention is to provide an apparatus for controlling the movement of sperms in the uterine tubes of a female patient.

In accordance with this object of the present invention, there is provided an apparatus for controlling a flow of sperms in an uterine tube of a female patient, the apparatus comprising: an implantable constriction device for gently constricting at least one portion of the uterine tube wall to at least partly constrict the uterine tube, a stimulation device for stimulating the wall portion of the uterine tube to further constrict and influence the uterine tube, and a control device for controlling the stimulation device to stimulate the uterine tube wall portion, as the constriction device constricts the uterine tube wall portion, to cause contraction of the uterine tube wall portion to further constrict and influence the flow of sperms in the uterine tube.

The present invention provides an advantageous combination of constriction and stimulation devices, which results in a two-stage influence on the sperms in the uterine tube. Thus, the constriction device may gently constrict the uterine tube wall by applying a relatively weak force against the wall portion, and the stimulation device may stimulate the constricted wall portion to achieve the desired final influence on the flow in the uterine tube. The phrase “gently constricting a portion of the uterine tube wall” is to be understood as constricting the wall portion without substantially hampering the blood circulation in the uterine tube wall.

However, it is possible the apparatus comprise either the constriction device or the stimulation device. Therefore all the embodiments described in this application should be understood to exists in three versions. Only stimulation device or only constriction device or the combination thereof. The term uterine tube of course relates to either one or preferable the two uterine tubes. The device may be used either to promote or prevent pregnancy, affecting the flow of sperm in two different directions, wherein preventing the sperm reaching the egg prevents pregnancy.

### The Invention Therefore Includes the Following Objects:

An apparatus:
for controlling a flow of sperms in an uterine tube of a female patient, the apparatus comprising:
a stimulation device for stimulating the wall portion of the uterine tube wall to constrict and influence the flow of sperm in an uterine tube, and
a control device for controlling the stimulation device to stimulate the uterine tube wall portion, to cause contraction of the uterine tube wall portion to constrict and influence the flow of sperms in the uterine tube.

The stimulation device may be combined with a constriction device described above in the all the embodiments described herein as well as use all the different embodiments related to the stimulation device described herein.

An apparatus:
for controlling a flow of sperms in an uterine tube of a female patient, the apparatus comprising:
an implantable constriction device for constricting at least one portion of the uterine tube wall to at least partly constrict the uterine tube, and
a control device for controlling the constriction device to constricts the uterine tube wall portion, to cause contraction of the uterine tube wall portion to influence the flow of sperms in the uterine tube.

The constriction device may be combined with a stimulation device described above in the all the embodiments described herein, as well as use all the different embodiments related to the constriction device described herein.

An apparatus, wherein said the constriction device is adapted to constrict the wall portion to at least restrict the flow in the uterine tube, and said the control device controls said the stimulation device to cause contraction of the constricted wall portion, so that the flow in the uterine tube is at least further restricted.

An apparatus, wherein the constriction device is adapted to constrict the wall portion to a constricted state in which the blood circulation in the constricted wall portion is substantially unrestricted and the flow in the uterine tube is at least restricted, and the control device controls the stimulation device to cause contraction of the wall portion, so that the flow in the uterine tube is at least further restricted when the wall portion is kept by the constriction device in the constricted state.

An apparatus, wherein the control device simultaneously controls the constriction device and the stimulation device.

Preferably, the stimulation device is adapted to stimulate different areas of the wall portion as the constriction device constricts the wall portion, and the control device controls the stimulation device to intermittently and individually stimulate the areas of the wall portion. This intermittent and individual stimulation of different areas of the wall portion of the organ allows tissue of the wall portion to maintain substantially normal blood circulation during the operation of the apparatus of the invention.

In most applications using the present invention, there will be daily adjustments of the implanted constriction device. Therefore, in a preferred embodiment of the invention, the constriction device is adjustable to enable adjustment of the constriction of the wall portion as desired, wherein the control device controls the constriction device to adjust the constriction of the wall portion. The control device may control the constriction and stimulation devices independently of each other, and simultaneously. Optionally, the control device may control the stimulation device to stimulate, or to not stimulate the wall portion while the control device controls the constriction device to change the constriction of the wall portion.

Initially, the constriction device may be calibrated by using the control device to control the stimulation device to stimulate the wall portion, while controlling the constriction device to adjust the constriction of the wall portion until the desired restriction of the flow of sperms in the uterine tube is obtained.

### Flow Restriction

The apparatus of the present invention is well suited for restricting the flow of sperms in the uterine tube of a female patient. Thus, in a principal embodiment of the invention, the constriction device is adapted to constrict the wall portion to at least restrict the flow in the uterine tube, and the control device controls the stimulation device to cause contraction of the constricted wall portion, so that the flow in the uterine tube is at least further restricted. Specifically, the constriction device is adapted to constrict the wall portion to a constricted state in which the blood circulation in the constricted wall portion is substantially unrestricted and the flow in the uterine tube is at least restricted, and the control device controls the stimulation device to cause contraction of the wall portion, so that the flow in the uterine tube is at least further restricted when the wall portion is kept by the constriction device in the constricted state.

The constriction and stimulation devices may be controlled to constrict and stimulate, respectively, to an extent that depends on the flow restriction that is desired to be achieved in a specific application of the apparatus of the invention. Thus, in accordance with a first flow restriction option, the control device controls the constriction device to constrict the wall portion, such that flow in the uterine tube is restricted but not stopped, and controls the stimulation device to stimulate the constricted wall portion to cause contraction thereof, such that flow of sperms in the uterine tube is further restricted but not stopped. More precisely, the control device may control the stimulation device in a first mode to stimulate the constricted wall portion to further restrict but not stop the flow in the uterine tube and to:
a) control the stimulation device in a second mode to cease the stimulation of the wall portion to increase the flow in the uterine tube; or
b) control the stimulation and constriction devices in the second mode to cease the stimulation of the wall portion and release the wall portion to restore the flow in the uterine tube.

In accordance with a second flow restriction option, the control device controls the constriction device to constrict the wall portion, such that flow in the uterine tube is restricted but not stopped, and controls the stimulation device to stimulate the constricted wall portion to cause contraction thereof, such that flow in the uterine tube is stopped. More precisely, the control device may control the stimulation device in a first mode to stimulate the constricted wall portion to further restrict but not stop the flow in the uterine tube and to:
a) control the stimulation device in a second mode to cease the stimulation of the wall portion to allow flow in the uterine tube; or
b) control the stimulation and constriction devices in the second mode to cease the stimulation of the wall portion and release the wall portion to restore the flow in the uterine tube.

In accordance with a third flow restriction option, the control device controls the constriction device to constrict the wall portion, such that the flow of sperms in the uterine tube is substantially stopped, and controls the stimulation device to stimulate the constricted wall portion to cause contraction thereof, such that the flow in the uterine tube is completely stopped. More precisely, the control device may control the stimulation device in a first mode to stimulate the constricted wall portion to completely stop the flow in the uterine tube and to:
a) control the stimulation device in a second mode to cease the stimulation of the wall portion to allow flow in the uterine tube; or
b) control the stimulation and constriction devices in the second mode to cease the stimulation of the wall portion and release the wall portion to restore the flow in the uterine tube.

For example, the third flow restriction option may be applied where the present invention is used for controlling sperm flow of a patient who has difficulties getting pregnant. Thus, the restriction and stimulation devices may be implanted on any part of the patient's uterine tubes. The control device may control the constriction device to gently flatten a portion of the uterine tubes to at least almost completely stop the sperm flow, and controls the stimulation device to stimulate the flattened portion to insure that the sperm flow is completely stopped. Since the control device controls the stimulation device to intermittently and individually stimulate the areas of the wall portion, as stated above, the risk of the implanted constriction device injuring the uterine tubes over time is significantly reduced or even eliminated, and it is insured that the effect of the stimulation is maintained over time. When the time for getting pregnant is at an optimum, the control device controls the constriction and stimulation devices to release the portion of the uterine tubes and cease the stimulation, whereby sperms may pass the portion of the uterine tubes.

Where the stimulation device stimulates the constricted wall portion to contract, such that the sperm flow in the uterine tube is stopped, the control device suitably controls the stimulation device to simultaneously and cyclically stimulate a first length of the constricted wall portion and a second length of the constricted wall portion, which is located downstream of the first length, wherein the control device controls the stimulation device to progressively stimulate the first length in the upstream direction of the uterine tube and to progressively stimulate the second length in the downstream direction of the uterine tube.

The control device may control the stimulation device to change the stimulation of the wall portion in response to a sensed physical parameter of the patient or functional parameter of the apparatus. For example, the control device may control the stimulation device to increase the intensity of the stimulation of the wall portion in response to a sensed pressure increase in the uterine tube, such that the flow in the uterine tube remains stopped. Any sensor for sensing a physical parameter of the patient, such as a pressure in the patient's body that relates to the pressure in the uterine tube may be provided, wherein the control device controls the stimulation device in response to signals from the sensor. Such a sensor may for example sense the pressure in the patient's abdomen, the pressure against the implanted constriction device or the pressure on the uterine tube wall of the bodily organ.

For example, a pressure sensor may be applied where the present invention is used for controlling sperm flow of a patient. Thus, the constriction and stimulation devices may be applied on the patient's uterine tubes.

In accordance with a fourth flow restriction option, the control device controls the constriction device to constrict the wall portion, such that the flow in the uterine tube is stopped. More precisely, the control device may control the constriction device in a first mode to constrict the constricted wall portion to stop the flow in the uterine tube and in a second mode to cease the constriction of the wall portion to restore flow in the uterine tube. In this case, the control device only controls the stimulation device to stimulate the wall portion when needed. A sensor for sensing a physical parameter of the patient's body that relates to the pressure in the uterine tube may be provided, wherein the control device controls the stimulation device in response to signals from the sensor. Such a physical parameter may be a pressure in the patient's abdomen and the sensor may be a pressure sensor.

In some applications of the invention, the implanted constriction device may be designed to normally keep the patient's wall portion of the uterine tubes in the constricted state. In this case, the control device may be used when needed, conveniently by the patient, to control the stimulation device to stimulate the constricted uterine tube wall portion, preferably while adjusting the stimulation intensity, to cause contraction of the wall portion, such that the flow in the uterine tube is at least further restricted or stopped, and to control the stimulation device to cease the stimulation. More precisely, the control device may:
a) control the stimulation device in a first mode to stimulate the constricted wall portion to further restrict the flow in the uterine tube, and control the stimulation device in a second mode to cease the stimulation of the wall portion to increase the flow in the uterine tube; or
b) control the stimulation device in a first mode to stimulate the constricted wall portion to stop the flow in the uterine tube, and control the stimulation device in a second mode to cease the stimulation of the wall portion to allow flow in the uterine tube.

Either the first mode or the second mode may be temporary.

The constriction device may include a plurality of separate constriction elements adapted to constrict any wall portions of a series of wall portions of the uterine tube wall, respectively. The control device may control the constriction device to activate the constriction elements in random or in accordance with a predetermined sequence. In this case, the stimulation device includes stimulation elements positioned on the constriction elements, wherein the control device controls the stimulation device to activate the stimulation elements to stimulate any wall portions of the series of wall portions constricted by the constriction elements to contract the organ to close the organ's uterine tube.

Alternatively, the control device controls the constriction device to activate the constriction elements to constrict all of the wall portions of the series of wall portions, and controls the stimulation device to activate the stimulation elements to stimulate any constricted wall portions in random or in accordance with a predetermined sequence to close the organ's uterine tube. The design of the constriction device in the form of a plurality of separate constriction elements makes possible to counteract growth of hard fibrosis where the constriction device is implanted.

### Movement of Sperms in a Uterine Tube

The apparatus can be used for actively moving the sperms in the uterine tube of a patient, as described in the embodiments listed below. Either to promote or prevent pregnancy.
1) The control device controls the constriction device to close the uterine tube, either at an upstream end or a downstream end of the wall portion, and then controls the constriction device to constrict the remaining part of the wall portion to move the sperms in the uterine tube.
1a) In accordance with a first alternative of the above noted embodiment (1), the control device controls the stimulation device to stimulate the wall portion as the constriction device constricts the remaining part of the wall portion.
1b) In accordance with a second alternative, the constriction device is adapted to constrict the wall portion to restrict but not stop the flow in the uterine tube. The control device controls the stimulation device to stimulate the wall portion constricted by the constriction device to close the uterine tube, either at an upstream end or a downstream end of the wall portion, and simultaneously controls the constriction device to increase the constriction of the wall portion to move the sperms in the uterine tube.
2) The constriction device is adapted to constrict the wall portion to restrict or vary the flow in the uterine tube, and the control device controls the stimulation device to progressively stimulate the constricted wall portion, in the downstream or upstream direction of the uterine tube, to cause progressive contraction of the wall portion to move the sperms in the uterine tube.
3) The control device controls the constriction device to vary the constriction of the different areas of the wall portion, such that the wall portion is progressively constricted in the downstream or upstream direction of the uterine tube to move the sperms in the uterine tube. The constriction device may include at least one elongated constriction element that extends along the wall portion, wherein the control device controls the elongated constriction element to progressively constrict the wall portion in the downstream or upstream direction of the uterine tube.
3a) In accordance with a preferred alternative of the above noted embodiment (3), the control device controls the stimulation device to progressively stimulate the constricted wall portion to cause progressive contraction thereof in harmony with the progressive constriction of the wall portion performed by the constriction device. Where the constriction device includes at least one elongated constriction element the control device controls the elongated constriction element to progressively constrict the wall portion in the downstream or upstream direction of the uterine tube. Suitably, the elongated constriction element comprises contact surfaces dimensioned to contact a length of the wall portion, when the constriction device constricts the wall portion, and the stimulation device comprises a plurality of stimulation elements distributed along the contact surfaces, such that the stimulation elements stimulate the different areas of the wall portion along the length of the wall portion, when the control device controls the stimulation device to stimulate the wall portion.
4) The constriction device is adapted to constrict any one of a series of wall portions of the uterine tube wall to at least restrict the flow in the uterine tube. The control device controls the constriction device to successively constrict the wall portions of the series of wall portions to move the sperms in the uterine tube in a peristaltic manner.
4a) In accordance with a first alternative of embodiment (4), the constriction device includes a plurality of constriction elements adapted to constrict the wall portions of the uterine tube wall, respectively. The control device controls the constriction device to activate the constriction elements one after the other, so that the wall portions of the series of wall portions are successively constricted along the organ, whereby the sperms in the uterine tube is moved.
4b) In accordance with a second alternative of embodiment (4), the constriction device includes at least one constriction element that is moveable along the wall of the organ to successively constrict the wall portions of the series of wall portions, wherein the control device controls the constriction device to cyclically move the constriction element along the wall portions of the series of wall portions. Preferably, the constriction device comprises a plurality of constriction elements, each of which is moveable along the wall of the organ to successively constrict the wall portions of the series of wall portions, wherein the control device controls the constriction device to cyclically move the constriction elements one after the other along the wall portions of the series of wall portions. Specifically, the constriction device includes a rotor carrying the constriction elements, and the control device controls the rotor to rotate, such that each constriction element cyclically constricts the wall portions of the series of wall portions. Each constriction element suitably comprises a roller for rolling on the wall of the organ to constrict the latter.
4c) In accordance with a preferred alternative of the above noted embodiment (4), the stimulation device stimulates any of the wall portions of the series of wall portions constricted by the constriction device, to close the uterine tube. Where the constriction device includes at least one constriction element, the stimulation device suitably includes at least one stimulation element positioned on the constriction element for stimulating the wall portion constricted by the constriction element to close the uterine tube.

Where the constriction device includes a plurality of constriction elements, the stimulation device suitably includes stimulation elements positioned on the constriction elements for stimulating the wall portions constricted by the constriction elements to close the uterine tube.
5) The constriction device is adapted to constrict any one of a series of wall portions of the uterine tube wall to restrict the flow in the uterine tube, wherein the constriction device includes a plurality of constriction elements adapted to constrict the wall portions of the uterine tube wall, respectively, and the stimulation device includes stimulation elements positioned on the constriction elements for stimulating the wall portions constricted by the constriction elements to close the uterine tube. The control device controls the constriction device to activate the constriction elements to constrict the wall portions of the series of wall portions without completely closing the organ's uterine tube, and controls the stimulation device to activate the stimulation elements to stimulate the wall portions one after the other, so that the wall portions of the series of wall portions are successively contracted along the organ to move the sperms in the uterine tube.
6) The constriction device comprises a first constriction element for constricting the wall portion at an upstream end thereof, a second constriction element for constricting the wall portion at a downstream end thereof, and a third constriction element for constricting the wall portion between the upstream and downstream ends thereof. The control device controls the first, second and third constriction elements to constrict and release the wall portion independently of one another. More specifically, the control device controls the first or second constriction element to constrict the wall portion at the upstream or downstream end thereof to close the uterine tube, and controls the third constriction element to constrict the wall portion between the upstream and downstream ends thereof, whereby the sperms contained in the wall portion between the upstream and downstream ends thereof is moved downstream or upstream in the uterine tube. Optionally, the control device controls the stimulation device to stimulate the wall portion between the upstream and downstream ends thereof, when the third constriction element constricts the wall portion.
6a) In accordance with a first alternative, the control device controls the first constriction element to constrict the wall portion at the upstream end thereof to restrict the flow in the uterine tube and controls the stimulation device to stimulate the constricted wall portion at the upstream end to close the uterine tube. With the uterine tube closed at the upstream end of the constricted wall portion, the control device controls the third constriction element to constrict the wall portion between the upstream and downstream ends thereof, and optionally controls the stimulation device to simultaneously stimulate the wall portion as the latter is constricted by the third constriction element. As a result, the sperms contained in the wall portion between the upstream and downstream ends thereof are moved downstream in the uterine tube.
6b) In accordance with a second alternative, the control device controls the second constriction element to constrict the wall portion at the downstream end thereof to restrict the flow in the uterine tube and controls the stimulation device to stimulate the constricted wall portion at the downstream end to close the uterine tube. With the uterine tube closed at the downstream end of the constricted wall portion, the control device controls the third constriction element to constrict the wall portion between the upstream and downstream ends thereof, and optionally controls the stimulation device to simultaneously stimulate the wall portion as the latter is constricted by the third constriction element. As a result, the sperms contained in the wall portion between the upstream and downstream ends thereof are moved upstream in the uterine tube.

In any of the above noted embodiments (1) to (6b), the stimulation device may stimulate the wall portion with electric pulses.

With the tubular shape of the uterine tubes, a particularly long wall portion thereof may be surgically prepared to extend in zigzag with adjacent walls stitched together by two parallel rows of stitches and with the adjacent walls cut through between the two rows of stitches. As a result, the uterine tube of this long wall portion of the organ can be significantly expanded. In this case, the constriction device of the apparatus is able to move a considerably larger volume of fluid each time it constricts the long wall portion of the organ.

The various solutions described above under the headline: “Flow restriction” to stop the flow in the uterine tube of the organ may also be used in any of the above noted embodiments (1a), (1b), (4a), (5), (6), (6a) and (6b).

To summarize a few preferred embodiments see below:

In accordance with an object any wall portions of a series of wall portions of the organ's tissue wall, respectively. In accordance with an alternative, the wall portions of the series of wall portions are constricted in random or in accordance with a predetermined sequence. In accordance with an alternative, the wall portions of the series of wall portions are successively constricted along the organ to move the sperm in the lumen of the patient's organ or to prevent the fluid and/or other bodily matter to move in the lumen of the patient's organ.

In accordance with an alternative, step (b) is performed by stimulating any constricted wall portions of the series of wall portions. In accordance with an alternative, the wall portions of the series of wall portions are constricted in random or in accordance with a predetermined sequence. In accordance with an alternative, wherein the wall portions of the series of wall portions are successively constricted along the organ to move the fluid and/or other bodily matter in the lumen of the patient's organ or to prevent the fluid and/or other bodily matter to move in the lumen of the patient's organ.

In accordance with an alternative, step (a) is performed by constricting any wall portions of a series of wall portions of the organ's tissue wall, respectively, wherein the wall portions of the series of wall portions are successively constricted without completely closing the organ's lumen, and step (b) is performed by stimulating the constricted wall portions, so that the wall portions of the series of wall portions are further constricted. In accordance with an alternative, the wall portions of the series of wall portions are constricted in random or in accordance with a predetermined sequence.

In accordance with an alternative, wherein the wall portions of the series of wall portions are successively constricted along the organ to move the fluid and/or other bodily matter in the lumen of the patient's organ or to prevent the fluid and/or other bodily matter to move in the lumen of the patient's organ.

In accordance with an alternative, step (a) is performed by constricting all of the wall portions of the series of wall portions, and step (b) is performed by stimulating any constricted wall portions so that the wall portions of the series of wall portions are further constricted.

In accordance with an alternative, the wall portions of the series of wall portions are further constricted by the stimulation device in random or in accordance with a predetermined sequence.

In accordance with an alternative, the wall portions of the series of wall portions are successively further constricted by the stimulation device along the organ to move the fluid and/or other bodily matter in the lumen of the patient's organ or to prevent the fluid and/or other bodily matter to move in the lumen of the patient's organ.

In accordance with an alternative for all applicable alternatives, step (a) and step (b) are performed independently of each other or in accordance with an alternative, step (a) and step (b) are performed simultaneously.

### Stimulation

When stimulating neural or muscular tissue there is a risk of injuring or deteriorating the tissue over time, if the stimulation is not properly performed. The apparatus of the present invention is designed to reduce or even eliminate that risk. Thus, in accordance with the present invention, the control device controls the stimulation device to intermittently stimulate different areas of the wall portion of the uterine tube, such that at least two of these areas are stimulated at different points of time that is, the stimulation is shifted from one area to another area over time. In addition, the control device controls the stimulation device, such that an area of the different areas that currently is not stimulated has time to restore substantially normal blood circulation before the stimulation device stimulates the area again. Furthermore, the control device controls the stimulation device to stimulate each area during successive time periods, wherein each time period is short enough to maintain satisfactory blood circulation in the area until the lapse of the time period. This gives the advantage that the apparatus of the present invention enables continuous stimulation of the wall portion of the uterine tube to achieve the desired flow control, while essentially maintaining over time the natural physical properties of the uterine tube without risking injuring the organ.

Also, by physically changing the places of stimulation on the organ overtime as described above it is possible to create an advantageous changing stimulation pattern on the uterine tube, in order to achieve a desired flow control.

The control device may control the stimulation device to stimulate one or more of the areas of the wall portion at a time, for example by sequentially stimulating the different areas. Furthermore, the control device may control the stimulation device to cyclically propagate the stimulation of the areas along the wall portion, preferably in accordance with a determined stimulation pattern. To achieve the desired reaction of the uterine tube wall during the stimulation thereof, the control device may control the stimulation device to, preferably cyclically, vary the intensity of the stimulation of the wall portion.

In a preferred embodiment of the invention, the control device controls the stimulation device to intermittently stimulate the areas of the wall portion with pulses that preferably form pulse trains. At least a first area and a second area of the areas of the wall portion may be repeatedly stimulated with a first pulse train and a second pulse train, respectively, such that the first and second pulse trains over time are shifted relative to each other. For example, the first area may be stimulated with the first pulse train, while the second area is not stimulated with the second pulse train, and vice versa. Alternatively, the first and second pulse trains may be shifted relative to each other, such that the first and second pulse trains at least partially overlap each other.

The pulse trains can be configured in many different ways. Thus, the control device may control the stimulation device to vary the amplitudes of the pulses of the pulse trains, the duty cycle of the individual pulses of each pulse train, the width of each pulse of the pulse trains, the length of each pulse train, the repetition frequency of the pulses of the pulse trains, the repetition frequency of the pulse trains, the number of pulses of each pulse train, and/or the off time periods between the pulse trains. Several pulse trains of different configurations may be employed to achieve the desired effect.

In case the control device controls the stimulation device to vary the off time periods between pulse trains that stimulate the respective area of the wall portion, it is also possible to control each off time period between pulse trains to last long enough to restore substantially normal blood circulation in the area when the latter is not stimulated during the off time periods.

### Electric Stimulation

In accordance with a preferred embodiment of the invention, the stimulation device is an electrically powered stimulation device that electrically stimulates the uterine tube wall portion of the patient's bodily organ, preferably with electric pulses. This embodiment is particularly suited for applications in which the wall portion includes muscle fibers that react to electrical stimula. In this embodiment, the control device controls the stimulation device to stimulate the wall portion with electric pulses preferably in the form of electric pulse trains, when the wall portion is in the constricted state, to cause contraction of the wall portion. Of course, the configuration of the electric pulse trains may be similar to the above described pulse trains and the control device may control the stimulation device to electrically stimulate the different areas of the wall of the uterine tube in the same manner as described above.

The electric stimulation device suitably comprises at least one, preferably a plurality of electrical elements, such as electrodes, for engaging and stimulating the wall portion with electric pulses. Optionally, the electrical elements may be placed in a fixed orientation relative to one another. The control device controls the electric stimulation device to electrically energize the electrical elements, one at a time, or groups of electrical elements at a time. Preferably, the control device controls the electric stimulation device to cyclically energize each element with electric pulses. Optionally, the control device may control the stimulation device to energize the electrical elements, such that the electrical elements are energized one at a time in sequence, or such that a number or groups of the electrical elements are energized at the same time. Also, groups of electrical elements may be sequentially energized, either randomly or in accordance with a predetermined pattern.

The electrical elements may form any pattern of electrical elements. Preferably, the electrical elements form an elongate pattern of electrical elements, wherein the electrical elements are applicable on the patient's wall of the uterine tube, such that the elongate pattern of electrical elements extends lengthwise along the wall of the uterine tube, and the elements abut the respective areas of the wall portion. The elongate pattern of electrical elements may include one or more rows of electrical elements extending lengthwise along the wall of the uterine tube. Each row of electrical elements may form a straight, helical or zig-zag path of electrical elements, or any form of path. The control device may control the stimulation device to successively energize the electrical elements longitudinally along the elongate pattern of electrical elements in a direction opposite to, or in the same direction as that of, the flow in the patient's uterine tube.

Optionally, the control device may control the stimulation device to successively energize the electrical elements from a position substantially at the center of the constricted wall portion towards both ends of the elongate pattern of electrical elements. Where the uterine tube is to be kept closed for a relatively long time, the control device may control the stimulation device to energize the electrical elements, such that energized electrical elements form two waves of energized electrical elements that simultaneously advance from the center of the constricted wall portion in two opposite directions towards both ends of the elongate pattern of electrical elements. Such waves of energized electrical elements can be repeated over and over again without harming the uterine tube and without moving fluid or gas in any direction in the uterine tube.

The control device suitably controls the stimulation device to energize the electrical elements, such that the electrical elements currently energized form at least one group of adjacent energized electrical elements. In accordance with a first alternative, the elements in the group of energized electrical elements form one path of energized electrical elements. The path of energized electrical elements may extend at least in part around the patient's uterine tubes. In a second alternative, the elements of the group of energized electrical elements may form two paths of energized electrical elements extending on mutual sides of the patient's uterine tube, preferably substantially transverse to the flow direction in the uterine tube. In a third alternative, the elements of the group of energized electrical elements may form more than two paths of energized electrical elements extending on different sides of the patient's uterine tube, preferably substantially transverse to the flow direction in the patient's uterine tube.

In accordance with a preferred embodiment of the invention, the electrical elements form a plurality of groups of elements, wherein the groups form a series of groups extending along the patient's organ in the flow direction in the patient's uterine tube. The electrical elements of each group of electrical elements may form a path of elements extending at least in part around the patient's uterine tube. In a first alternative, the electrical elements of each group of electrical elements may form more than two paths of elements extending on different sides of the patient's uterine tube, preferably substantially transverse to the flow direction in the patient's uterine tube. The control device may control the stimulation device to energize the groups of electrical elements in the series of groups in random, or in accordance with a predetermined pattern. Alternatively, the control device may control the stimulation device to successively energize the groups of electrical elements in the series of groups in a direction opposite to, or in the same direction as that of, the flow in the patient's uterine tube, or in both the directions starting from a position substantially at the center of the constricted wall portion. For example, groups of energized electrical elements may form advancing waves of energized electrical elements, as described above; that is, the control device may control the stimulation device to energize the groups of electrical elements, such that energized electrical elements form two waves of energized electrical elements that simultaneously advance from the center of the constricted wall portion in two opposite directions towards both ends of the elongate pattern of electrical elements.

A structure may be provided for holding the electrical elements in a fixed orientation. Although the structure may be separate from the constriction device, it is preferable that the structure is integrated in the constriction device, which is a practical design and facilitates implantation of the constriction and stimulation devices. Where the electrical elements form an elongate pattern of electrical elements, the structure may be applicable on the patient's uterine tube such that the elongate pattern of electrical elements extends along the uterine tube in the same direction as that of the flow in the patient's uterine tube and the elements abut the respective areas of the wall portion of the uterine tube.

### Thermal Stimulation

In another embodiment of the invention, the stimulation device thermally stimulates the wall portion of the uterine tube. Thus, the control device may control the stimulation device to cool the wall portion, when the wall portion is constricted, to cause contraction of the wall portion. For example, the constriction device may constrict the wall portion to at least restrict the flow in the uterine tube, and the control device may control the stimulation device to cool the constricted wall portion to cause contraction thereof, such that the flow in the uterine tube is at least further restricted, or further restricted but not stopped, or stopped. Alternatively, the control device may control the stimulation device to heat the wall portion, when the wall portion is constricted and contracted, to cause expansion of the wall portion. Where the wall portion includes a blood vessel, the control device may control the stimulation device to cool the blood vessel to cause contraction thereof, or heat the blood vessel to cause expansion thereof. Where applicable, thermal stimulation may be practiced in any of the embodiments of the present invention, and the thermal stimulation may be controlled in response to various sensors, for example strain, motion or pressure sensors.

Sensor Controlled Constriction and/or Stimulation Device

As mentioned above, the apparatus may comprise at least one implantable sensor, wherein the control device controls the constriction device and/or the stimulation device in response to signals from the sensor. Generally, the sensor directly or indirectly senses at least one physical parameter of the patient, or at least one functional parameter of the apparatus, or at least one functional parameter of a medical implant in the patient.

Many different kinds of sensor for sensing physical parameters may be used. For example motion sensors for sensing uterine tube motion, i.e. natural contractions, uterine tube contractions, pressure sensors for sensing pressure in the organ, strain sensors for sensing strain of the uterine tube, flow sensors for sensing fluid flow in the uterine tube of the organ, spectro-photometrical sensors, Ph-sensors for sensing acidity or alkalinity of the fluid in the uterine tube, oxygen-sensors sensors for sensing the oxygen content of the fluid in the uterine tube, or sensors for sensing the distribution of the stimulation on the stimulated uterine tube. Any conceivable sensors for sensing any other kind of useful physical parameter may be used.

Many different kinds of sensors that sense functional parameters of the apparatus may also be used for the control of the constriction device and/or the stimulation device. For example sensors for sensing electric parameters of implanted electric components of the apparatus, or sensors for sensing the performance of implanted motors of the apparatus.

The sensor may comprise a pressure sensor for sensing as the physical parameter a pressure in the patient's body that relates to the pressure in the uterine tube of the patient, wherein the control device controls the constriction device and/or stimulation device to change the constriction of the patient's wall portion in response to the pressure sensor sensing a predetermined value of measured pressure.

Alternatively, or in combination with the pressure sensor, a position sensor may be provided for sensing as the physical parameter the orientation of the patient with respect to the horizontal. The position sensor may be a biocompatible version of what is shown in U.S. Pat. Nos. 4,942,668 and 5,900,909, incorporated herein by reference. For example, the control device may control the constriction device and/or stimulation device to change the constriction of the patient's uterine tube wall portion in response to the position sensor sensing that the patient has assumed a substantially horizontal orientation, i.e. that the patient is lying down.

The above described sensors may be used in any of the embodiments of the invention, where applicable.

The control device may control the constriction device and/or stimulation device to change the constriction of the patient's wall portion in response to the time of day. For that purpose the control device may include a clock mechanism for controlling the constriction device and/or stimulation device to change the constriction of the patient's wall portion to increase or decrease the influence on the flow in the uterine tube during different time periods of the day. In case a sensor of any of the above-described types for sensing a physical or functional parameter is provided, either the clock mechanism is used for controlling the constriction device and/or stimulation device provided that the parameter sensed by the sensor does not override the clock mechanism, or the sensor is used for controlling the constriction device and/or stimulation device provided that the clock mechanism does not override the sensor. Suitably, the control device produces an indication, such as a sound signal or displayed information, in response to signals from the sensor.

The control device may comprise an implantable internal control unit that directly controls the constriction device and/or stimulation device in response to signals from the sensor. The control device may further comprise a wireless remote control adapted to set control parameters of the internal control unit from outside the patient without mechanically penetrating the patient. At least one of the control parameters, which is settable by the wireless remote control, is the physical or functional parameter. Suitably, the internal control unit includes the above mentioned clock mechanism, wherein the wireless remote control also is adapted to set the clock mechanism.

Alternatively, the control device may comprise an external control unit outside the patient's body for controlling the constriction device and/or stimulation device in response to signals from the sensor.

### Adjustable Constriction Device

In several alternative embodiments of the invention, the constriction device is adjustable. In these embodiments, there is an operation device for operating the adjustable constriction device to change the constriction of the patient's uterine tube wall portion, and the constriction and stimulation devices form a constriction/stimulation unit. Preferably, the constriction and stimulation devices of the constriction/stimulation unit are integrated in a single piece suitable for implantation. The constriction device of the unit comprises contact surfaces dimensioned to contact a length of a uterine tube wall portion, and the stimulation device of the unit comprises a plurality of stimulation elements provided on and distributed along the contact surfaces. When the control device controls the stimulation device to stimulate the wall portion, the stimulation elements stimulate different areas of the wall portion along the length of the wall portion. The stimulation elements preferably comprise electric elements, as described above, for stimulating the wall portion with electric pulses. However, in most applications of the present invention, other kinds of stimulations, such as thermal stimulation, could be suitable to employ.

The operation device operates the adjustable constriction device of the constriction/stimulation unit in a manner that depends on the design of the constriction device, as will be explained by the following examples of embodiments. 1) The constriction device comprises at least two elongated clamping elements having the contact surfaces and extending along the wall portion on different sides of the organ, and the operation device operates the clamping elements to clamp the uterine tube wall portion between the clamping elements to constrict the wall portion.
2) The constriction device comprises one elongate clamping element having the contact surfaces and extending along the wall portion on one side of the uterine tube, and the operation device operates the clamping element to clamp the wall portion between the clamping element and the bone or tissue of the patient to constrict the wall portion.
3) The constriction device comprises at least two engagement elements having the contact surfaces and positioned on different sides of the uterine tube, and the operation device rotates the engagement elements, such that the engagement elements engage and constrict the wall portion of the uterine tube.
4) The constriction device comprises at least two articulated clamping elements having the contact surfaces and positioned on different sides of the uterine tube, and the operation device moves the clamping elements towards each other to clamp the wall portion of the uterine tube between the clamping elements, to constrict the wall portion.
5) The constriction device comprises at least two separate clamping elements having the contact surfaces, at least one of the clamping elements being pivoted, such that it may turn in a plane in which the loop of the constriction member extends, and the operation device turns the pivoted clamping element to change the size of the constriction opening.
6) The constriction device comprises at least one elongated constriction member having the contact surfaces, and forming means for forming the constriction member into at least a substantially closed loop around the uterine tube, wherein the loop defines a constriction opening. The operation device operates the constriction member in the loop to change the size of the constriction opening.
   6a) The elongated constriction member comprises a belt having the contact surfaces, and the operation device operates the belt to change the longitudinal extension of the belt in the loop to change the size of the constriction opening. The forming means may form the constriction member or belt into a loop having at least one predetermined size.
   6b) The elongated constriction member is operable to change the size of the constriction opening, such that the outer circumferential confinement surface of the constriction device is changed, or, alternatively, is unchanged.
   6c) The elongated constriction member is elastic and varies in thickness as seen in a cross-section there through, and is operable to turn around the longitudinal extension of the constriction member.
   6d) The elongated constriction member comprises two substantially or partly semi-circular frame elements having the contact surfaces and hinged together, such that the semi-circular elements are swingable relative to each other from a fully open state in which they substantially or partly form a circle to a fully folded state in which they substantially form a semi-circle.
7) The constriction device is adapted to bend the wall portion of the uterine tube to constrict the latter.

In the above noted embodiments (1) to (7), it is important that the constriction device is designed to constrict the length of the uterine tube wall portion of the patient's uterine tube. For this purpose, the constriction device may include two or more of the described constriction elements/members to be applied in a row along the length of the wall portion, wherein the row extends in the direction of flow in the uterine tube of the uterine tube. Preferably, such constriction elements/members are non-inflatable and mechanically operable or adjustable.

In the above noted embodiments (1) to (7), the operation device may either mechanically or hydraulically adjust the constriction device of the constriction/stimulation unit. Also, the operation device may comprise an electrically powered operation device for operating the constriction device. For many applications of the present invention, the operation device suitably operates the constriction device, such that the through-flow area of the uterine tube assumes a size in the constricted state that enables the stimulation device to contract the wall portion such that the flow in the uterine tube is stopped.

### Mechanical Operation

Where the operation device mechanically operates the constriction device of the constriction/stimulation unit, it may be non-inflatable. Furthermore, the operation device may comprise a servo system, which may include a gearbox. The term “servo system” encompasses the normal definition of a servo mechanism, i.e., an automatic device that controls large amounts of power by means of very small amounts of power, but may alternatively or additionally encompass the definition of a mechanism that transfers a weak force acting on a moving element having a long stroke into a strong force acting on another moving element having a short stroke. Preferably, the operation device operates the constriction device in a non-magnetic and/or non-manual manner. A motor may be operatively connected to the operation device. The operation device may be operable to perform at least one reversible function and the motor may be capable of reversing the function.

### Hydraulic Operation

Where the operation device hydraulically operates the constriction device of the constriction/stimulation unit, it includes hydraulic means for adjusting the constriction device.

In an embodiment of the invention, the hydraulic means comprises a reservoir and an expandable/contractible cavity in the constriction device, wherein the operation device distributes hydraulic fluid from the reservoir to expand the cavity, and distributes hydraulic fluid from the cavity to the reservoir to contract the cavity. The cavity may be defined by a balloon of the constriction device that abuts the uterine tube wall portion of the patient's uterine tube, so that the patient's wall portion is constricted upon expansion of the cavity and released upon contraction of the cavity.

Alternatively, the cavity may be defined by a bellows that displaces a relatively large contraction element of the constriction device, for example a large balloon that abuts the wall portion, so that the patient's wall portion is constricted upon contraction of the bellows and released upon expansion of the bellows. Thus, a relatively small addition of hydraulic fluid to the bellows causes a relatively large increase in the constriction of the wall portion. Such a bellows may also be replaced by a suitably designed piston/cylinder mechanism.

Where the hydraulic means comprises a cavity in the constriction device, the apparatus can be designed in accordance with the options listed below.
1) The reservoir comprises first and second wall portions, and the operation device displaces the first and second wall portions relative to each other to change the volume of the reservoir, such that fluid is distributed from the reservoir to the cavity, or from the cavity to the reservoir.
   1a) The first and second wall portions of the reservoir are displaceable relative to each other by at least one of a magnetic device, a hydraulic device or an electric control device.
2) The operation device comprises a pump for pumping fluid between the reservoir and the cavity.
   2a) The pump comprises a first activation member for activating the pump to pump fluid from the reservoir to the cavity and a second activation member for activating the pump to pump fluid from the cavity to the reservoir.
   2a1) The first and second activation members are operable by manual manipulation thereof.
   2a2) At least one of the activation members operates when subjected to an external predetermined pressure.
   2a3) At least one of the first and second activating members is operable by magnetic means, hydraulic means, or electric control means.
2b) The apparatus comprises a fluid conduit between the pump and the cavity, wherein the reservoir forms part of the conduit. The conduit and pump are devoid of any non-return valve. The reservoir forms a fluid chamber with a variable volume, and the pump distributes fluid from the chamber to the cavity by a reduction in the volume of the chamber and withdraws fluid from the cavity by an expansion of the volume of the chamber. The apparatus further comprises a motor for driving the pump, wherein the pump comprises a movable wall of the reservoir for changing the volume of the chamber.

In all of the above noted embodiments 1 to 2b where the hydraulic means comprises an expandable cavity in the constriction device, the cavity can be exchanged by a cylinder/piston mechanism for adjusting the constriction device. In this case, the operation device distributes hydraulic fluid between the reservoir and the cylinder/piston mechanism to adjust the constriction device.

In a special embodiment of the invention, the operation device comprises a reverse servo operatively connected to the hydraulic means. The term “reverse servo” is to be understood as a mechanism that transfers a strong force acting on a moving element having a short stroke into a weak force acting on another moving element having a long stroke; i.e., the reverse function of a normal servo mechanism. Thus, minor changes in the amount of fluid in a smaller reservoir could be transferred by the reverse servo into major changes in the amount of fluid in a larger reservoir. The reverse servo is particularly suited for manual operation thereof.

Preferably, the reverse servo comprises an expandable servo reservoir containing servo fluid and a fluid supply reservoir hydraulically connected to the servo reservoir to form a closed conduit system for the servo fluid. The expandable servo reservoir has first and second wall portions, which are displaceable relative to each other in response to a change in the volume of the expandable servo reservoir.

In accordance with a first alternative, the first and second wall portions of the servo reservoir are operatively connected to the hydraulic means. The reverse servo distributes fluid between the fluid supply reservoir and the expandable servo reservoir to change the volume of the servo reservoir, whereby the hydraulic means is operated to adjust the constriction device.

In accordance with a second alternative, there is provided an implantable main reservoir containing a predetermined amount of hydraulic fluid, wherein the reverse servo is operable to distribute hydraulic fluid between the main reservoir and the hydraulic means to adjust the constriction device. More specifically, the main reservoir is provided with first and second wall portions operatively connected to the first and second wall portions of the expandable servo reservoir, such that the volume of the main reservoir is changed when the volume of the expandable servo reservoir is changed. Thus, when the reverse servo distributes servo fluid between the fluid supply reservoir and the expandable servo reservoir to change the volume of the main reservoir, hydraulic fluid is distributed from the main reservoir to the hydraulic means, or from the hydraulic means to the main reservoir. Advantageously, the servo and main reservoirs are dimensioned, such that when the volume of the servo reservoir is changed by a relatively small amount of servo fluid, the volume of the main reservoir is changed by a relatively large amount of hydraulic fluid.

In both of the above-described alternatives, the fluid supply reservoir may have first and second wall portions, which are displaceable relative to each other to change the volume of the fluid supply reservoir to distribute servo fluid between the fluid supply reservoir and the expandable servo reservoir. The first and second wall portions of the fluid supply reservoir may be displaceable relative to each other by manual manipulation, a magnetic device, a hydraulic device, or an electric control device to change the volume of the fluid supply reservoir to distribute servo fluid between the fluid supply reservoir and the expandable servo reservoir.

In all of the above noted embodiments 1 to 2b where the hydraulic means comprises an expandable cavity in the constriction device, or in embodiments where the hydraulic means comprises a hydraulically operable mechanical construction, the operation device may include the reverse servo described above. In a further embodiment of the invention, the hydraulic means include first and second hydraulically interconnected expandable/contractible reservoirs. The first reservoir is operatively connected to the constriction device, such that the constriction device changes the constriction of the patient's wall portion upon expansion or contraction of the first reservoir. By changing the volume of the second reservoir hydraulic fluid is distributed between the two reservoirs, so that the first reservoir is either expanded or contracted. This embodiment requires no non-return valve in the fluid communication conduits between the two reservoirs, which is beneficial to long-term operation of the hydraulic means.

Alternatively, the hydraulic means may include first and second hydraulically interconnected piston/cylinder mechanisms instead of the first and second reservoirs described above. The first piston/cylinder mechanism is operatively connected to the constriction device, such that the constriction device changes the constriction of the patient's wall portion upon operation of the first piston/cylinder mechanism. By operating the second piston/cylinder mechanism hydraulic fluid is distributed between the two piston/cylinder mechanisms, so that the first piston/cylinder mechanism adjusts the constriction device.

Where the constriction device does not include an expandable/contractible cavity, the constriction device may comprise at least two elongated clamping elements having the above-mentioned contact surfaces and extending along the wall portion on different sides of the uterine tube. The hydraulic means, which may include the reverse servo described above, hydraulically moves the elongated clamping elements towards the wall portion to constrict the wall portion. For example, the constriction device may have hydraulic chambers in which the clamping elements slide back and forth, and the hydraulic means may also include a pump and an implantable reservoir containing hydraulic fluid. The pump distributes hydraulic fluid from the reservoir to the chambers to move the clamping elements against the wall portion, and distributes hydraulic fluid from the chambers to the reservoir to move the clamping elements away from the wall portion.

### Design of Control Device

The control device suitably controls the constriction/stimulation unit from outside the patient's body. Preferably, the control device is operable by the patient. For example, the control device may comprise a manually operable switch for switching on and off the constriction/stimulation unit, wherein the switch is adapted for subcutaneous implantation in the patient to be manually or magnetically operated from outside the patient's body. Alternatively, the control device may comprise an external control unit in the form of a hand-held wireless remote control, which is conveniently operable by the patient to switch on and off the constriction/stimulation unit. The wireless remote control may also be designed for application on the patient's body like a wristwatch. Such a wristwatch type of remote control may emit a control signal that follows the patient's body to implanted signal responsive means of the apparatus.

Where the control device wirelessly controls the constriction/stimulation unit from outside the patient's body, the wireless control function is preferably performed in a non-magnetic manner, i.e., the control device controls the constriction device of the constriction/stimulation unit in a non-magnetic manner. The patient may use the remote control to control the constriction/stimulation unit to adjust the stimulation intensity and/or adjust the constriction of the wall portion. The wireless remote control may comprise at least one external signal transmitter or transceiver and at least one internal signal receiver or transceiver implantable in the patient.

The wireless remote control preferably transmits at least one wireless control signal for controlling the constriction/stimulation unit. The control signal may comprise a frequency, amplitude, phase modulated signal or a combination thereof, and may be an analogue or a digital signal, or a combination of an analogue and digital signal. The remote control may transmit an electromagnetic carrier wave signal for carrying the digital or analogue control signal. Also the carrier signal may comprise digital, analogue or a combination of digital and analogue signals.

Any of the above control signals may comprise wave signals, for example a sound wave signal, an ultrasound wave signal, an electromagnetic wave signal, an infrared light signal, a visible light signal, an ultra violet light signal, a laser light signal, a microwave signal, a radio wave signal, an x-ray radiation signal or a gamma radiation signal. Alternatively, the control signal may comprise an electric or magnetic field, or a combined electric and magnetic field.

As mentioned above, the control signal may follow the patient's body to implanted signal responsive means of the apparatus.

The control device may include a programmable internal control unit, such as a microprocessor, implantable in the patient for controlling the constriction/stimulation unit. The control device may further include an external control unit intended to be outside the patient's body, wherein the internal control unit is programmable by the external control unit. For example, the internal control unit may be programmable for controlling the constriction/stimulation unit over time, suitably in accordance with an activity schedule program. The apparatus may comprise an external data communicator and an implantable internal data communicator communicating with the external data communicator, wherein the internal communicator feeds data related to the constriction/stimulation unit back to the external data communicator or the external data communicator feeds data to the internal data communicator.

### Source of Energy

The present invention also presents a solution for supplying energy for use in connection with the operation of the constriction/stimulation unit. Thus, in a broad sense, the present invention provides an apparatus for controlling a flow of sperms in a uterine tube formed by a uterine tube wall of a patient's uterine tube, wherein the apparatus comprises an implantable constriction device for gently constricting a portion of the uterine tube wall to influence the flow in the uterine tube, a stimulation device for intermittently and individually stimulating different areas of the wall portion, as the constriction device constricts the wall portion, to cause contraction of the wall portion to further influence the flow in the uterine tube, wherein the constriction and stimulation devices form an operable constriction/stimulation unit, a source of energy, and a control device operable from outside the patient's body to control the source of energy to release energy for use in connection with the operation of the constriction/stimulation unit. In a simple form of the invention, the source of energy, such as a battery or accumulator, is implantable in the patient's body.

### Transmission of Wireless Energy

In a more sophisticated form of the invention, which is preferable, the source of energy is external to the patient's body and the control device controls the external source of energy to release wireless energy. In this sophisticated form of the invention, the apparatus comprises an energy-transmission device that transmits the released wireless energy from outside the patient's body to inside the patient's body. Among many things the wireless energy may comprise electromagnetic energy, an electric field, an electromagnetic field or a magnetic field, or a combination thereof, or electromagnetic waves. The energy-transmission device may transmit wireless energy for direct use in connection with the operation of the constriction/stimulation unit, as the wireless energy is being transmitted. For example, where an electric motor or pump operates the constriction device, wireless energy in the form of a magnetic or an electromagnetic field may be used for direct power of the motor or pump.

Thus, the motor or pump is running directly during transmission of the wireless energy. This may be achieved in two different ways: a) using a transforming device implanted in the patient to transform the wireless energy into energy of a different form, preferably electric energy, and powering the motor or pump with the transformed energy, or b) using the wirelessly transmitted energy to directly power the motor or pump. Preferably wireless energy in the form of an electromagnetic or magnetic field is used to directly influence specific components of the motor or pump to create kinetic energy for driving the motor or pump. Such components may include coils integrated in the motor or pump, or materials influenced by magnetic fields, or permanent magnets, wherein the magnetic or electromagnetic field influences the coils to generate a current for driving the motor or pump, or influences the material or permanent magnets to create kinetic energy for driving the motor or pump.

Preferably, the energy-transmission device transmits energy by at least one wireless signal, suitably a wave signal. The wave signal may comprise an electromagnetic wave signal including one of an infrared light signal, a visible light signal, an ultra violet light signal, a laser signal, a microwave signal, a radio wave signal, an x-ray radiation signal, and a gamma radiation signal. Alternatively, the wave signal may comprise a sound or ultrasound wave signal. The wireless signal may be a digital or analogue signal, or a combination of a digital and analogue signal.

### Transforming Wireless Energy

In accordance with a particular embodiment of the invention, an implantable energy-transforming device is provided for transforming wireless energy of a first form transmitted by the energy-transmission device into energy of a second form, which typically is different from the energy of the first form. The constriction/stimulation unit is operable in response to the energy of the second form. For example, the wireless energy of the first form may comprise sound waves, whereas the energy of the second form may comprise electric energy. In this case, the energy-transforming device may include a piezo-electric element for transforming the sound waves into electric energy. Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transforming device may function differently from or similar to the energy-transmission device. In a special embodiment, the energy-transforming device comprises at least one element, such as at least one semiconductor, having a positive region and a negative region, when exposed to the energy of the first form transmitted by the energy-transmission device, wherein the element is capable of creating an energy field between the positive and negative regions, and the energy field produces the energy of the second form. More specifically, the element may comprise an electrical junction element, which is capable of inducing an electric field between the positive and negative regions when exposed to the energy of the first form transmitted by the energy-transmission device, whereby the energy of the second form comprises electric energy.

The energy-transforming device may transform the energy of the first form directly or indirectly into the energy of the second form. An implantable motor or pump for operating the constriction device of the constriction/stimulation unit may be provided, wherein the motor or pump is powered by the energy of the second form. The constriction device may be operable to perform at least one reversible function and the motor may be capable of reversing the function. For example, the control device may shift polarity of the energy of the second form to reverse the motor.

The energy-transforming device may directly power the motor or pump with the transformed energy, as the energy of the second form is being transformed from the energy of the first form. Preferably, the energy-transforming device directly operates the constriction/stimulation unit with the energy of the second form in a non-magnetic, non-thermal or non-mechanical manner.

Normally, the constriction/stimulation unit comprises electric components that are energized with electrical energy. Other implantable electric components of the apparatus may be at least one voltage level guard or at least one constant current guard. Therefore, the energy-transforming device may transform the energy of the first form into a direct current or pulsating direct current, or a combination of a direct current and pulsating direct current. Alternatively, the energy-transforming device may transform the energy of the first form into an alternating current or a combination of a direct and alternating current.

The apparatus may comprise an internal source of energy implantable in the patient for supplying energy for the operation of the constriction/stimulation unit. The apparatus may further comprise an implantable switch operable to switch from an “off” mode, in which the internal source of energy is not in use, to an “on” mode, in which the internal source of energy supplies energy for the operation of the constriction/stimulation unit, and/or for energizing implanted electronic components of the apparatus. The switch may be operable by the energy of the first form transmitted by the energy-transmission device or by the energy of the second form supplied by the energy-transforming device. The described switch arrangement reduces power consumption of the apparatus between operations.

The internal source of energy may store the energy of the second form supplied by the energy-transforming device. In this case, the internal source of energy suitably comprises an accumulator, such as at least one capacitor or at least one rechargeable battery, or a combination of at least one capacitor and at least one rechargeable battery. Where the internal source of energy is a rechargeable battery it may be charged only at times convenient for the patient, for example when the patient is sleeping. Alternatively, the internal source of energy may supply energy for the operation of the constriction/stimulation unit but not be used for storing the energy of the second form. In this alternative, the internal source of energy may be a battery and the switch described above may or may not be provided.

Suitably, the apparatus comprises an implantable stabilizer for stabilizing the energy of the second form. Where the energy of the second form is electric energy the stabilizer suitably comprises at least one capacitor.

The energy-transforming device may be designed for implantation subcutaneously in the abdomen, thorax or cephalic region of the patient. Alternatively, it may be designed for implantation in an orifice of the patient's body and under the mucosa or intramuscularly outside the mucosa of the orifice.

Although the constriction/stimulation unit in the embodiments described above is designed as a single piece, which is most practical for implantation, it should be noted that as an alternative the constriction device and stimulation device could be designed as separate pieces. Any one of the constriction and stimulation units described above may alternatively be replaced by two or more separate constriction/stimulation elements, which are controlled independently of one another.

In one embodiment, the apparatus comprises an implantable constriction device for constricting each one of the female's uterine tubes to restrict the passageway thereof, and a control device for controlling the constriction device to constrict the uterine tube such that an sperm appearing in the passageway of the uterine tube is prevented from entering the uterine cavity, and to release the uterine tube such that an sperm existing in the passageway of the uterine tube is allowed to enter the uterine cavity. The constriction device may gently constrict at least one portion of the uterine tube wall of the uterine tube to restrict the passageway thereof, and an implantable stimulation device may be provided for stimulating the uterine tube wall portion, wherein the control device controls the stimulation device to stimulate the uterine tube wall portion, as the constriction device constricts the uterine tube wall portion, to cause contraction of the uterine tube wall portion to further restrict the passageway of the uterine tube.

Alternatively, the sperm flow control apparatus comprises an implantable constriction device for gently constricting at least one portion of the uterine tube wall of each one of the female's uterine tubes to restrict the passageway thereof, a stimulation device for stimulating the uterine tube wall portion of the uterine tube, and a control device for controlling the stimulation device to stimulate the uterine tube wall portion, as the constriction device constricts the uterine tube wall portion, to cause contraction of the uterine tube wall portion to further restrict the passageway of the uterine tube to prevent an sperm existing in the uterine tube from entering the uterine cavity.

Alternatively, the sperm flow control apparatus comprises an implantable stimulation device for stimulating a portion of the uterine tube wall of each one of the female's uterine tubes, and a control device for controlling the stimulation device to stimulate the uterine tube wall portion of the uterine tube to cause contraction of the uterine tube wall portion, such that the passageway of the uterine tube is restricted to prevent an sperm appearing in the uterine tube from entering the uterine cavity, and to cease stimulating the uterine tube wall portion of the uterine tube to allow an sperm existing in the passageway of the uterine tube to enter the uterine cavity.

The present invention also provides a method for using an apparatus as described above to control a flow of sperms in a female patient's uterine tube, the method comprising:
providing a wireless remote control adapted to control the constriction device and/or stimulation device from outside the patient's body, and
operating the wireless remote control by the patient, when the patient wants to influence the flow of sperms in the uterine tube.

The present invention also provides a method for controlling a flow of sperms in a female patient's uterine tube, the method comprising:
a) gently constricting at least one portion of the uterine tube wall to influence the flow in the uterine tube, and
b) stimulating the constricted wall portion to cause contraction of the wall portion to further influence the flow of sperm in the uterine tube.

### General Method

The present invention also provides a method for controlling a flow of sperms in a female patient's uterine tube, the method comprising:
stimulating the wall portion to cause contraction of the wall portion to influence the flow of sperm in the uterine tube.

The stimulation device may be combined with a constriction device described above in the all the method embodiments described herein as well as use all the different method embodiments related to the stimulation device described herein.

The present invention also provides a method for controlling a flow of sperms in a female patient's uterine tube, the method comprising: constricting at least one portion of the uterine tube wall to influence the flow of sperm in the uterine tube.

The constriction device may be combined with a stimulation device in the all the method embodiments described herein, as well as use all the different method embodiments related to the constriction device and methods described herein.

### Laparoscopic Method

The method for controlling a flow of sperm in a lumen formed by a tissue wall of a patient's uterine tube, the method comprising the steps of:
inserting a needle like tube into a cavity of the patient's body,
using the needle like tube to fill the cavity with gas thereby expanding the 1 cavity,
placing at least two laparoscopical trocars in the patient's body,
inserting a camera through one of the trocars into the cavity,
inserting a dissecting tool through any of the trocar and dissecting an area of at least one portion of the tissue wall of the uterine tube,
placing a constriction device and a stimulation device in the dissected area in operative engagement with the uterine tube,
using the constriction device to gently constrict the wall portion of the uterine tube to influence the flow in the lumen, and
using the stimulation device to stimulate the constricted wall portion to cause contraction of the wall portion to further influence the flow in the lumen.

The method for controlling a flow of sperm in a lumen formed by a tissue wall of a patient's uterine tube, the method comprising the steps of:
inserting a needle like tube into a cavity of the patient's body,
using the needle like tube to fill the cavity with gas thereby expanding the cavity,
placing at least two laparoscopical trocars in the patient's body,
inserting a camera through one of the trocars into the cavity,
inserting a dissecting tool through any of the trocar and dissecting an area of at least one portion of the tissue wall of the uterine tube,
placing a stimulation device in the dissected area in operative engagement with the uterine tube, and
using the stimulation device to stimulate the wall portion to cause contraction of the wall portion to influence the flow in the lumen.

The method for controlling a flow of sperm in a lumen formed by a tissue wall of a patient's uterine tube, the method comprising the steps of:
inserting a needle like tube into a cavity of the patient's body,
using the needle like tube to fill the cavity with gas thereby expanding the cavity,
placing at least two laparoscopical trocars in the patient's body,
inserting a camera through one of the trocars into the cavity,
inserting a dissecting tool through any of the trocar and dissecting an area of at least one portion of the tissue wall of the uterine tube,
placing a constriction device in the dissected area in operative engagement with the uterine tube,
using the constriction device to constrict the wall portion of the uterine tube to influence the flow in the lumen.

The method for controlling a flow of uterine tube in a lumen formed by a tissue wall of a patient's uterine tube, the method comprising the steps of:
cutting the skin of the patient,
inserting a dissecting tool and dissecting an area of at least one portion of the tissue wall of the uterine tube,
placing a constriction device and a stimulation device in the dissected area in operative engagement with the uterine tube,
using the constriction device to gently constrict the wall portion of the uterine tube to influence the flow in the lumen, and
using the stimulation device to stimulate the constricted wall portion to cause contraction of the wall portion to further influence the flow in the lumen.

The method for controlling a flow of sperm in a lumen formed by a tissue wall of a patient's uterine tube, the method comprising the steps of:
cutting the skin of the patient,
inserting a dissecting tool and dissecting an area of at least one portion of the tissue wall of the uterine tube,
placing a stimulation device in the dissected area in operative engagement with the uterine tube, and
using the stimulation device to stimulate the wall portion to cause contraction of the wall portion to influence the flow in the lumen.

The method for controlling a flow of sperm in a lumen formed by a tissue wall of a patient's uterine tube, the method comprising the steps of:
cutting the skin of the patient,
inserting a dissecting tool and dissecting an area of at least one portion of the tissue wall of the organ,
placing a constriction device in the dissected area in operative engagement with the uterine tube, and
using the constriction device to constrict the wall portion of the uterine tube to influence the flow in the lumen.

The method according to any one of the six method embodiment above, wherein the cavity comprising; at least one of an abdominal cavity, a cavity in the pelvic region, a cavity in human soft tissue such as muscle, fat and fibrotic tissue.

The further method embodiments could used to any of the above:

The method according, further comprising implanting a powered operation device for operating the constriction device.

The method, wherein the operation device comprises a powered hydraulic operation device.

The method, wherein the operation device comprises an electrically powered operation device.

The method, wherein the operation device comprises an electric motor.

The method, further comprising transmitting wireless energy for powering the operation device, and when desired to influence the flow in the patient's uterine tube, powering the operation device with the transmitted energy to operate the constriction device.

The method, further comprising implanting a source of energy in the patient, providing an external source of energy, controlling the external source of energy to release wireless energy, transforming the wireless energy into storable energy, non-invasively charging the implanted source of energy with the transformed energy, and controlling the implanted source of energy from outside the patient's body to release energy for use in connection with the operation of the constriction device and/or stimulation device.

The method, wherein the wireless energy is transformed into a storable energy different from the wireless energy.

### Preventing Flow or Promoting Flow

First, the apparatus, wherein the flow influence device comprising one or more restriction devices, adapted to contract more than one tissue wall portion, wherein the one or more restriction devices is adapted to restrict a series of wall portions of the uterine tube's tissue wall.

Second, the apparatus wherein the restriction device comprising a constriction device for restricting the wall portions of the series of wall portions, adapted to restrict the wall portions of the series of wall portions in random or in accordance with a predetermined sequence.

Third, the apparatus, wherein the restriction device comprising a constriction device for restricting the wall portions of the series of wall portions and further comprising a stimulation device for further restricting any constricted wall portions of the series of wall portions, in random or in accordance with a predetermined sequence.

Fourth, the apparatus, wherein the restriction device, comprising a constriction device, for constricting without completely closing the uterine tube's lumen, and further comprising a stimulation device for stimulating the constricted wall portions one after the other, so that the wall portions of the series of wall portions are successively restricted along the uterine tube, in random or in accordance with a predetermined sequence.

Fifth, the apparatus, wherein the restriction device, comprising a stimulation device for stimulating any wall portion of a series of wall portions to restrict the wall portions of the series of wall portions in random or in accordance with a predetermined sequence.

Sixth, the apparatus, wherein the restriction device, comprising a constriction device for restricting all of the wall portions of the series of wall portions, and a stimulating device for stimulating any restricted wall portions in random or in accordance with a predetermined sequence to close the uterine tube's lumen.

The apparatus, as described in all six objects above, wherein the control device is adapted to control the one or more restriction devices to restrict the wall portions of the series of wall portions at least two at a time with the restrictions placed spaced apart, wherein the control device is adapted to successively restrict the wall portions of the series of wall portions along the uterine tube to move the sperm in the lumen of the patient's uterine tube or to prevent the sperm to move in the lumen of the patient's uterine tube.

The apparatus, as described in all six objects above, wherein the control device is adapted to successively restrict the wall portions of the series of wall portions along the uterine tube to move the sperm in the lumen of the patient's uterine tube or to prevent the sperm to move in the lumen of the patient's uterine tube.

### Feedback Related to the Wireless Energy

The following embodiments are related to feedback information related to an energy balance either comparing;
a) the amount of energy received by the internal energy source compared to the energy used by the constriction device and/or stimulation device, or
b) The amount of energy received by the internal energy source and the amount of energy transmitted by the external energy source.

Several alternatives of the method of the present invention are disclosed below and may except being correlated directly to the constriction device and/or stimulation device also be included in the operating method. These methods are valid for use both with the stimulation device and constriction device separate or in combination.

A method for controlling the transmission of wireless energy comprising an internal energy source, wherein said wireless energy is transmitted from an external energy source located outside the patient and is received by the internal energy source located inside the patient, the internal energy source being connected to the constriction device and/or stimulation device for directly or indirectly supplying received energy thereto, the method comprising the steps of:
determining an energy balance between the energy received by the internal energy source and the energy used for the constriction device and/or stimulation device, and
controlling the transmission of wireless energy from the external energy source, based on the determined energy balance.

A method, wherein the wireless energy is transmitted inductively from a primary coil in the external energy source to a secondary coil in the internal energy receiver.

A method, wherein a change in said energy balance is detected, and the transmission of wireless energy is controlled based on said detected energy balance change.

A method, wherein a difference is detected between energy received by said internal energy receiver and energy used for a medical device, and the transmission of wireless energy is controlled based on said detected energy difference.

A method, wherein the amount of transmitted wireless energy is decreased if the detected energy balance change implies that the energy balance is increasing, or vice versa.

A method, wherein the decrease/increase of energy transmission corresponds to a detected change rate.

A method, wherein the amount of transmitted wireless energy is decreased if the detected energy difference implies that the received energy is greater than the used energy, or vice versa.

A method, wherein the decrease/increase of energy transmission corresponds to the magnitude of said detected energy difference.

A method, wherein the energy used for the constriction device and/or stimulation device is stored in at least one energy storage device of the device.

A method, wherein substantially all the energy used for the constriction device and/or stimulation device i is consumed to operate the device.

A method, wherein the energy is consumed after being stabilized in at least one energy stabilizing unit of the device.

A method, wherein the energy used for the constriction device and/or stimulation device is stored in at least one energy storage device of the device.

A method, wherein substantially all the energy used for the constriction device and/or stimulation device i is consumed to operate the device.

A method, wherein the energy is consumed after being stabilized in at least one energy stabilizing unit of the device.

A method of controlling transmission of wireless energy supplied to at least one of the constriction and stimulation devices, comprising an internal energy source located inside the patient, connected to the constriction device and/or stimulation device for directly or indirectly supplying received energy thereto, the method comprising the steps of:
determining an energy balance between the energy sent by the external energy source and the energy received by the internal energy source, and
controlling the transmission of wireless energy from the external energy source, based on the determined energy balance.

A method, wherein the wireless energy is transmitted inductively from a primary coil in the external energy source to a secondary coil in the internal energy receiver.

A method, wherein a change in said energy balance is detected, and the transmission of wireless energy is controlled based on said detected energy balance change.

A method, wherein a difference is detected between the energy sent by the external energy source and the energy received by said internal energy receiver, and the transmission of wireless energy is controlled based on said detected energy difference.

A method, wherein the amount of transmitted wireless energy is decreased if the detected energy balance change implies that the energy balance is increasing, or vice versa.

A method, wherein the decrease/increase of energy transmission corresponds to a detected change rate.

A method, wherein the amount of transmitted wireless energy is decreased if the detected energy difference implies that the received energy is greater than the used energy, or vice versa.

A method, wherein the decrease/increase of energy transmission corresponds to the magnitude of said detected energy difference.

A method of controlling transmission of wireless energy, wherein said wireless energy being transmitted by means of a primary coil in the external energy source and received inductively by means of a secondary coil in an internal energy source, the internal energy source being connected to the medical device for directly or indirectly supplying received energy thereto, wherein feedback control information (S) is transferred from the secondary coil to the primary coil by switching the secondary coil on and off to induce a detectable impedance load variation in the primary coil encoding the feedback control information, wherein the feedback control information relates to the energy received by the internal energy source and is used for controlling the transmission of wireless energy from the external energy source.

The apparatus according to claim 284, wherein the external energy source further comprises an electronic circuit for comparing the feedback information with the amount of energy transmitted by the external energy source.

The method, wherein the electronic circuit comprises an analyzer analyzing the amount of energy being transmitted and receiving the feedback information related to the amount of energy received in the receiver, and determining the energy balance by comparing the amount of transmitted energy and the feedback information related to the amount of received energy.

The method, wherein the external energy source is adapted to use said feedback information adjusting the level of said transmitted energy.

A method of controlling transmission of wireless energy, wherein said wireless energy being transmitted by means of a primary coil in an external energy source and received inductively by means of a secondary coil in an internal energy source, the internal energy receiver being connected to the medical device for directly or indirectly supplying received energy thereto, wherein feedback control information (S) is transferred from the secondary coil to the primary coil by switching the secondary coil on and off to induce a detectable impedance load variation in the primary coil encoding the feedback control information, where the feedback control information relates to said energy balance.

### R: Assisting Control for Lubrication of a Synovial Joint

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control of an implantable medical device for lubrication of a synovial joint. Examples of such devices for lubrication of joints will now be described.

An implantable medical device for lubrication of a synovial joint having a joint cavity is provided. The implantable device comprises a solid lubricant and a feeding device, wherein said feeding device is adapted to feed said solid lubricant into the joint cavity for lubricating the synovial joint.

According to one embodiment, the solid lubricant is adapted to be placed within an implantable cartridge having an opening into the joint cavity. An inner diameter of the opening could have substantially the same diameter as the inner diameter of said cartridge.

In some embodiments, the solid lubricant could have thixotropic or shear thinning properties, such that the viscosity of said solid lubricant is reduced when said solid lubricant is exposed to strain in the joint cavity.

According to other embodiments, the solid lubricant comprises high-molecular weight hyaluronic acid, which could be crosslinked high-molecular weight hyaluronic acid or hyaluronic acids of at least two different high-molecular weights, crosslinked to form a semisolid or solid gel.

According to another embodiments, the solid lubricant comprises a crosslinking agent chosen from 1, 2, 3, 4-diepoxybutane, divinyl sulfone.

The solid lubricant could be a hydrophilic polymer chosen from synthetic and natural polysaccharides, which could be selected from a group consisting of: hydroxyethyl cellulose, carboxymethyl cellulose, xanthan gum, chondroitin sulfate, heparin, protein, sulfated protein, synthetic water-soluble polymers.

According to one embodiment, the protein comprises a protein selected from a group consisting of: collagen, elastin, albumin, and globulin.

According to one embodiment, the sulfated protein could comprise a sulfated protein selected from a group consisting of: keratin sulfate, and sulfated aminoglycosaminoglycans.

According to one embodiment, the synthetic water-soluble polymer is a synthetic water-soluble polymer selected from a group consisting of: polyvinyl alcohol, co-polymers of polyvinyl alcohol, and co-polymers of poly-(hydroxethyl) methacrylate.

According to yet another embodiment, the medical device is adapted to be implanted in the area of the hip joint, such that said solid lubricant can be inserted into the joint cavity of the hip joint.

The medical device could be adapted to at least partially be implanted in the caput femur, such that the feeding device can feed the solid lubricant into the hip joint cavity, towards the acetabulum.

According to yet another embodiment, the implantable device is adapted to be inserted into a bore in the femoral bone, which could be a bore from the lateral side of the femoral bone, in the region of the major trochanter, or a bore in the pelvis, such that the feeding device can feed the solid lubricant into the hip joint cavity, towards the caput femur. The medical device could for example be adapted to be inserted into the bore in the pelvis, from the acetabulum side of the pelvic bone or from the abdominal side of the pelvic bone.

According to yet another embodiment, the implantable medical device is adapted to be implanted in the area of the knee joint, such that said solid lubricant can be inserted into the joint cavity of the knee joint.

According to yet another embodiment, the medical device is adapted to at least partially be implanted distally in the femoral bone, such that the feeding device can feed the solid lubricant into the knee joint cavity, towards the tibia bone.

According to yet another embodiment, the implantable device is adapted to be inserted into a bore in the distal portion of the femoral bone.

According to yet another embodiment, the medical device is adapted to at least partially be implanted proximally in the tibia bone, such that the feeding device can feed the solid lubricant into the knee joint cavity, towards the femoral bone.

According to yet another embodiment, the implantable device is adapted to be inserted into a bore in the proximal portion of the tibia bone.

According to yet another embodiment, the medical device is adapted to be implanted in the area of the shoulder joint, such that the solid lubricant can be inserted into the joint cavity of the shoulder joint.

According to yet another embodiment, the medical device is adapted to at least partially be implanted in the scapula bone, such that the feeding device can feed the solid lubricant into the shoulder joint cavity, towards the humerus bone, or implanted in the humerus bone, such that the feeding device can feed the solid lubricant into the shoulder joint cavity, towards the scapula bone.

According to yet another embodiment, the medical device further comprises a retention member for retaining the medical device inside of the bore, the retention member comprises at least one bone contacting portion adapted to press on the bone of the inside of the bore for retaining said medical device in the bore. The retention member could be comprises at least one spring member adapted to exert force on said at least one bone contacting portion.

The feeding device could according to one embodiment comprises an energized feeding device, which could comprise a motor.

In other embodiments, the feeding device comprises an elastic member, which could be a spring member or a member of elastic material.

According to yet another embodiment, the feeding is adapted to be powered by a pressurized gaseous fluid.

According to yet another embodiment, the medical device is adapted to, at least partially, be placed in a prosthesis comprising at least one joint surface being adapted for implantation.

The medical device could comprise a cartridge being adapted to be exchanged when said solid lubrication housed inside said cartridge has ended.

In yet another embodiment, the medical device further comprises an implantable sleeve adapted to be placed within a bone of the patient, and further adapted to receive said implantable medical device.

A method for improving the lubrication of a synovial joint of a patient is further provided. The method comprises the steps of drilling or punching a chamber in a bone of the patient, such that an opening of the chamber is located in the joint cavity, and implanting a medical device into the chamber comprising a solid lubricant.

In one embodiment, the method further comprises the step of implanting a feeding device adapted to feed said solid lubricant into the joint cavity for lubricating the synovial joint.

According to some embodiments the solid lubricant is housed within a cartridge, and the step of implanting the medical device could comprise the step of implanting said cartridge into the chamber.

In some embodiments, the step of drilling or punching a chamber in a bone of the patient could comprise the step of drilling or punching a chamber in a bone in the area of the hip joint, such that said solid lubricant can be inserted into the joint cavity of the hip joint. The area of the hip joint could be in the caput femur or in pelvis.

According to one embodiment, the step of implanting said cartridge into the chamber could comprise the step of implanting the cartridge into a bore created in the caput femur, such that the feeding device can feed the solid lubricant into the hip joint cavity, towards the acetabulum.

According to yet another embodiment, the step of implanting the cartridge into a bore could comprise the step of implanting the medical device into the bore from the lateral side of the femoral bone, in the region of the major trochanter.

The step of implanting the cartridge into the chamber could comprise the step of implanting the cartridge into pelvis from the abdominal side of the pelvis, such that the feeding device can feed the solid lubricant into the hip joint cavity, towards the caput femur.

In yet another embodiment, the step of drilling or punching a chamber in a bone of the patient could comprise the step of drilling or punching a chamber in a bone in the area of the knee joint, such that said solid lubricant can be inserted into the joint cavity of the knee joint.

The step drilling or punching a chamber in a bone of the patient comprises the step of drilling or punching a chamber in the femoral bone or the tibia bone.

In yet another embodiment, the step of implanting the cartridge into the chamber could comprise the step of implanting the cartridge into a bore created in the femoral bone, such that the feeding device can feed the solid lubricant into the knee joint cavity.

The step of implanting the cartridge into the chamber could comprise the step of implanting the cartridge into a bore created in the tibia bone, such that the feeding device can feed the solid lubricant into the knee joint cavity.

The step of drilling or punching a chamber in a bone of the patient could comprise the step of drilling or punching a chamber in a bone in the area of the shoulder joint, such that said solid lubricant can be inserted into the joint cavity of the shoulder joint, and the step of drilling or punching a chamber in a bone of the patient could comprise the step of drilling or punching a chamber in the humerus bone or in the scapula bone.

In yet other embodiments, the step of implanting the cartridge into the chamber could comprise the step of implanting said cartridge into a bore created in the humerus bone, such that the feeding device can feed the solid lubricant into the shoulder joint cavity.

The step of implanting the cartridge into the chamber could comprise the step of implanting the cartridge into a bore created in the scapula bone, such that the feeding device can feed the solid lubricant into the shoulder joint cavity.

According to yet another embodiment, the method further comprises the step of implanting a retention member for retaining the medical device inside of said chamber. The step of implanting the retention member could comprise the step of implanting the retention member such that the retention member presses on the bone of the inside of the bore for retaining said medical device in the bore.

The step of implanting a feeding device could comprise the step of implanting an energized feeding device, which in a further step could be connected.

The method could further comprise the step of implanting a prosthesis comprising at least one joint surface being adapted for implantation.

In yet another embodiment, the method could comprise the steps of creating an incision in the patient, removing the implanted medical device from the patient, and inserting a new medical device, and suturing or stapling the incision.

Please note that any embodiment or part of embodiment, feature, method, associated system, part of system described herein may be combined in any way.

Please note that any embodiment or part of embodiment as well as any method or part of method could be combined in any way. All examples herein should be seen as part of the general description and therefore possible to combine in any way in general terms. Please note that the description in general should be seen as describing both of an apparatus and a method.

It is an object of the present invention to provide an artificial valve for implantation into a mammal blood vessel which overcomes drawbacks of known such artificial valves.

### S: Artificial Heart Valve

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating an artificial heart valve, examples of such an artificial heart valves will now be described.

This object of the present invention is achieved by means of an artificial valve for implantation in a mammal body, in or adjacent to a mammal blood vessel. The artificial valve comprises a casing and an opening and closing mechanism, with at least part of the opening and closing mechanism being a first moving part adapted to make an opening and a closing movement relative to the casing, the movements comprising movements to assume an open and a closed position for opening and closing, respectively, of the blood flow through said blood vessel, as well as positions between said open and closed positions. The closing movement of the first moving part is initiated and carried out as the result of a predefined threshold value being reached by a physical parameter of the mammal or a functional parameter of a device used by the mammal, the physical or functional parameter being one or more of the following:
the blood pressure on an inner or an outer side of the valve or the difference in blood pressure between an inner and an outer side of the valve in its closed position,
the blood flow at a defined point in the circulatory system of the mammal,
a physical parameter which is related to the contraction of a muscle at a defined point in the mammal,
a body generated parameter related to the contraction of the mammal's heart muscle,
a device generated signal related to the contraction of the mammal's heart muscle.

In the artificial valve of the invention, the opening and closing mechanism is arranged to cause the moving part to initiate and carry out its closing movement by means of giving the moving part kinetic energy in a plurality of steps.

In one embodiment of the artificial valve of the invention, the opening and closing mechanism is also arranged to cause the moving part to initiate and carry out its opening movement by means of giving the moving part kinetic energy in a plurality of steps.

In one embodiment of the artificial valve of the invention, the physical parameter of the mammal which reaches a predefined threshold value is the blood pressure on an inner or an outer side of the valve or the difference in blood pressure between an inner and an outer side of the valve in its closed position, said threshold value being 5 mmHg or greater.

In one embodiment of the artificial valve of the invention, the physical parameter of the mammal which reaches a predefined threshold value is the blood pressure on an inner or an outer side of the valve or the difference in blood pressure between an inner and an outer side of the valve in its closed position, said threshold value being 10 mmHg or greater.

In one embodiment of the artificial valve of the invention, the closing mechanism is arranged to cause the moving part to carry out movement between two desired positions by means of giving the moving part kinetic energy in a plurality of steps.

In one embodiment of the artificial valve of the invention, the closing mechanism also comprises one or more magnets and one or more coils which are adapted to interact to cause a closing movement of the closing mechanism. In one such embodiment of the artificial valve of the invention, the one or more magnets are adapted to receive energy from the one or more coils as at least a first and a second pulse, with a time delay between said pulses in order to give the moving part kinetic energy in said plurality of steps

In one embodiment, the artificial valve comprises at least two magnets, and the closing mechanism is adapted to receive said first and second pulses as affecting different magnets or different groups of magnets.

In one embodiment of the artificial valve of the invention, the closing mechanism comprises a coil which is adapted to be energized stepwise with two or more energy pulses so as to cause said movement of the closing mechanism by means of affecting the magnet or magnets.

In one embodiment of the artificial valve of the invention, the closing mechanism comprises a plurality of coils which are adapted to be energized stepwise so as to cause said movement of the closing mechanism by means of affecting the magnet or magnets.

In one embodiment of the valve of the invention, the first moving part is adapted to move to assume an open or a closed position as well as positions in between said open and closed positions. Suitably, in this embodiment, the valve also comprises first and second hinges arranged in the casing, about which hinges the first moving part is arranged to be able to move to assume an open or a closed position as well as positions in between said open and closed positions.

In one embodiment, the closing mechanism of the valve also comprises a second moving part, and the first and second moving parts are adapted to move to assume a closed and an open position as well as to positions in between said open and closed positions, in order to close or limit the blood flow through the blood vessel. Suitably, in this embodiment, the first and second moving parts are movably hinged about respective first and second hinges in the casing, and can move about these hinges to assume an open or a closed position as well as positions in between said open and closed positions.

In one embodiment, the closing mechanism of the valve also comprises a second and a third moving part, and the first, second and third moving parts are adapted to move to assume a closed and an open position as well as positions in between said open and closed positions in order to close or limit the blood flow through the blood vessel. Suitably, in this embodiment, the first, second and third moving parts are movably hinged about respective first and second hinges in the casing, and can move about these hinges in order to assume an open or a closed position as well as positions in between said open and closed positions.

In one embodiment, the closing mechanism of the valve comprises an additional three or more moving parts, and the moving parts of the valve are adapted to move to assume a closed and an open position as well as positions in between said open and closed positions in order to close or limit the blood flow through the blood vessel. Suitably, in this embodiment, each of the moving parts is movably hinged about respective first and second hinges in said casing, and can move about these hinges in order to assume an open or a closed position as well as positions in between said open and closed positions.

Suitably, in the embodiments with two or more moving parts, the moving parts come together to form a cupola in the closed position, and also suitably, the first and second hinges of at least one of said moving parts are positioned at or adjacent to a meeting point of the moving parts. In addition, in these embodiments, the first and second hinges of at least one of said moving parts are placed at substantially opposite distal ends of said moving part along the casing.

In the embodiments described above, the closing mechanism can, as an alternative, be adapted to be powered in its movements to the opening and/or closed position in part or entirely by means of a power source which is external to the blood vessel. In such embodiments, the opening and/or closing then becomes an active measure, i.e. a measure which involves the supply of power from a source which is external to the blood vessel, as opposed to a passive measure which does not need the supply of external power.

In another embodiment, the closing mechanism of the valve comprises an elongated essentially flat plate which is adapted to, when the valve is arranged in or adjacent to an opening in said blood vessel, move into this opening in a direction which is essentially perpendicular to the blood vessel in order to limit or close the blood flow through said vessel.

In one embodiment with the plate, an outer wall of the casing is suitably concavely curved in order to essentially coincide with the outer shape of a blood vessel, and the curved outer wall also comprises an opening for the plate, through which opening the plate can move in its movements. The tolerance between the dimensions of the opening and the plate are then such that the movements of the plate are enabled, but also such that leakage of blood between the plate and the opening is essentially eliminated.

In the “plate embodiment”, the casing also suitably comprises at least a first curved protruding part in order to surround at least part of the circumference of a blood vessel, in order to enable attachment of the valve to the blood vessel.

In another embodiment, the casing comprises at least a first and a second curved protruding part for surrounding at least part of the circumference of a blood vessel, in order to enable attachment of the valve to the blood vessel.

The valve of the “plate embodiment” can also, in one embodiment, comprise a detachable part for attachment to the casing or to one or more protruding parts of the casing, so that the valve will completely surround a blood vessel by means of at least one protruding part and said detachable part and/or by means of an outer wall of the valve. In the “plate embodiment”, the closing mechanism is adapted to be powered in its movements to the opening and/or closed position in part or entirely by means of a power source external to the blood vessel, so that the opening and/or closing is an active measure, i.e. involving the supply of power from a source which is external to the blood vessel.

In the “plate embodiment”, the valve can also comprise a biasing mechanism, for biasing the plate to an open position.

The valve also suitably comprises a receiving device for receiving a closing signal, and for supplying this closing signal to the closing mechanism, which in turn is adapted to close upon reception of said signal.

The closing signal may be received by the receiving device from a source external to the valve, or it may be received from a sensor which is comprised in the valve. In both of these embodiments, the signal is supplied as the result of a parameter reaching a certain threshold value at which the valve should initiate its closing movement.

It is an object of the present invention to provide an artificial valve for implantation into a mammal blood vessel which overcomes drawbacks of known such artificial valves.

This object is obtained by means of an artificial valve for implantation in a mammal aorta or heart as an auxiliary aortic valve in addition to an aortic valve. The artificial valve comprises at least a first moving part which is adapted to be able to move to assume an open and a closed position for opening and closing, respectively, of the blood flow through a blood vessel, and the artificial valve also comprises a casing. In the artificial valve, the at least one first moving part is movably attached to said casing, and the artificial valve is adapted to let the at least one moving part initiate its movement to the open position at a level of blood pressure on a blood supplying side of the valve which is at least 5 mm Hg higher than the mammal's diastolic aortic blood pressure on the other side of the valve.

In one embodiment, the artificial valve is adapted to let the at least one moving part initiate its movement to said open position at a level of blood pressure on a blood supplying side of the valve which is at least 10 mm Hg higher than the mammal's diastolic aortic blood pressure on the other side of the valve.

In one embodiment, the artificial valve is adapted to let the at least one moving part initiate the movement to the closed position at a point in the mammal's heart cycle which is delayed compared to a normal aortic valve by at least ⅓ of the mammal's heart's diastolic phase.

In one embodiment, the artificial valve comprises a passive resistance mechanism by means of which a barrier force is created which corresponds to said level of blood pressure, with the at least one moving part being adapted to be opened by the blood flow at said level of blood pressure, the artificial valve being adapted to let said at least one first moving part move freely after the barrier force has been reached.

In one embodiment, the passive resistance mechanism of the artificial valve is arranged to offer resistance until said predetermined level of the barrier force is reached.

In one embodiment, the passive resistance mechanism of the artificial valve is arranged to only offer resistance in the opening movement of said at least one first moving part.

In one embodiment, the artificial valve comprises at least a first and a second moving part which are adapted to assume an open and a closed position for opening and closing, respectively, of the blood flow through the vessel. The valve also comprises a casing, and the moving parts of the inventive valve are movably attached to the casing.

In one embodiment of the valve of the invention, the moving parts come together to form a cupola in the closed position of the valve.

By means of this cupola shape, the valve will exhibit a higher degree of strength in the closed position than known valves.

Suitably, this embodiment of the inventive valve also comprises a third moving part, which is adapted to assume an open and a closed position for opening and closing, together with said first and second parts respectively, the blood flow through the blood vessel, and to come together with the first and second parts to form a cupola in the closed position of the valve.

In one embodiment, the valve comprises two or more additional moving parts, so that there are four or more moving parts altogether, which are adapted to together assume an open and a closed position for opening and closing the blood flow through the blood vessel, and to come together to form a cupola in the closed position of the valve.

In one embodiment of the inventive valve, each of the moving parts is movably hinged about respective first and second hinges in the casing, and each of said moving parts can move about these hinges to assume a closed and an open position as well as positions in between said open and closed positions, with the first and second hinges of at least one of the moving parts being positioned at or adjacent to a meeting point of two of said parts.

Also, in one embodiment, the first and second hinges of at least one of said moving parts are placed at substantially opposite distal ends of said moving part along the casing.

In one embodiment, the moving parts of the valve are adapted to be opened by the blood flow, as an alternative or complement to which the moving parts can be adapted to be closed to the cupola position by the blood flow.

Also, in one embodiment, the moving parts of the valve are adapted to be closed to the cupola position by blood pressure of a certain level.

In one embodiment, the valve comprises a resistance mechanism by means of which a barrier force of a predetermined level is needed to initiate the opening movement of the moving parts, so that the moving parts are given an initial moving resistance before the valve opens to allow blood to flow through the valve. Suitably but not necessarily, the barrier force is equal to that caused by a blood pressure of 10 mm Hg or more.

In one embodiment, the valve additionally comprises an operating mechanism which is adapted to power the moving parts in their movements to the closed and/or open position, so that the closing of the valve is active, and the powering is done at least in part by means of a power source which is external to the blood vessel. In one embodiment, the valve also comprises a receiving device which will receive a closing and/or an opening signal and will supply this signal to the operating mechanism which in turn will cause the moving parts to close and/or open upon its reception of said signal from the receiving device.

A method is thus provided for controlling transmission of wireless energy supplied to an electrically operable medical device implanted in a mammal patient. The wireless energy is transmitted by means of a primary coil in an external energy source located outside the patient and received inductively by means of a secondary coil in an internal energy receiver located inside the patient. The internal energy receiver is connected to the medical device for directly or indirectly supplying received energy thereto. Feedback control information is transferred from the secondary coil to the primary coil by switching the secondary coil on and off to induce a detectable impedance load variation in the primary coil encoding the feedback control information. The feedback control information relates to the energy for operating the medical device and is used for controlling the transmission of wireless energy from the external energy source.

An apparatus is also provided for controlling transmission of wireless energy supplied to an electrically operable medical device implanted in a mammal patient. The apparatus comprises an external energy source adapted to transmit the wireless energy by means of a primary coil when located outside the patient, and an internal energy receiver adapted to receive the transmitted wireless energy inductively by means of a secondary coil when located inside the patient, and to directly or indirectly supply received energy to the medical device. The internal energy receiver is further adapted to transfer feedback control information from the secondary coil to the primary coil in accordance with the above method.

The method and apparatus may be implemented according to different embodiments and features as follows:

In one embodiment, an internal control unit controls the on and off switching of the secondary coil, wherein the feedback control information may include at least one predetermined parameter relating to the received energy. The predetermined parameter may also be variable. The feedback control information may also relate to the received energy and require artificial intelligence to be generated. An implantable switch may be used to execute the on and off switching of the secondary coil as controlled by the internal control unit. The switch may be an electronic switch such as a transistor. Further, the internal control unit may comprise a memory for storing the transferred feedback control information.

In another embodiment, an internal control unit determines an energy balance between the energy received by the internal energy receiver and the energy used for the medical device, where the feedback control information relates to the determined energy balance. An external control unit then controls the transmission of wireless energy from the external energy source based on the determined energy balance and using the feedback control information.

In yet another embodiment, an external control unit determines the energy balance above based on the feedback control information, in that case comprising measurements relating to characteristics of the medical device, and controls the transmission of wireless energy from the external energy source based on the determined energy balance and using the feedback control information.

A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

In one alternative, substantially all energy used for the medical device is consumed to operate the medical device. In that case, the energy may be consumed after being stabilized in at least one energy stabilizing unit of the medical device.

In another alternative, substantially all energy used for the medical device is stored in the at least one energy storage device. In yet another alternative, the energy used for the medical device is partly consumed to operate the medical device and partly stored in the at least one energy storage device.

The energy received by the internal energy receiver may be stabilized by a capacitor, before the energy is supplied directly or indirectly to the medical device.

The difference between the total amount of energy received by the internal energy receiver and the total amount of consumed and/or stored energy may be directly or indirectly measured over time, and the energy balance can then be determined based on a detected change in the total amount difference.

The energy received by the internal energy receiver may further be accumulated and stabilized in an energy stabilizing unit, before the energy is supplied to the medical device. In that case, the energy balance may be determined based on a detected change followed over time in the amount of consumed and/or stored energy. Further, the change in the amount of consumed and/or stored energy may be detected by determining over time the derivative of a measured electrical parameter related to the amount of consumed and/or stored energy, where the derivative at a first given moment is corresponding to the rate of the change at the first given moment, wherein the rate of change includes the direction and speed of the change. The derivative may further be determined based on a detected rate of change of the electrical parameter.

The energy received by the internal energy receiver may be supplied to the medical device with at least one constant voltage, wherein the constant voltage is created by a constant voltage circuitry. In that case, the energy may be supplied with at least two different voltages, including the at least one constant voltage.

The energy received by the internal energy receiver may also be supplied to the medical device with at least one constant current, wherein the constant current is created by a constant current circuitry.

In that case, the energy may be supplied with at least two different currents including the at least one constant current.

The energy balance may also be determined based on a detected difference between the total amount of energy received by the internal energy receiver and the total amount of consumed and/or stored energy, the detected difference being related to the integral over time of at least one measured electrical parameter related to the energy balance. In that case, values of the electrical parameter may be plotted over time as a graph in a parameter-time diagram, and the integral can be determined from the size of the area beneath the plotted graph. The integral of the electrical parameter may relate to the energy balance as an accumulated difference between the total amount of energy received by the internal energy receiver and the total amount of consumed and/or stored energy.

The energy storage device in the medical device may include at least one of a rechargeable battery, an accumulator or a capacitor. The energy stabilizing unit may include at least one of an accumulator, a capacitor or a semiconductor adapted to stabilize the received energy.

When the energy received by the internal energy receiver is accumulated and stabilized in an energy stabilizing unit before energy is supplied to the medical device and/or energy storage device, the energy may be supplied to the medical device and/or energy storage device with at least one constant voltage, as maintained by a constant voltage circuitry. In that case, the medical device and energy storage device may be supplied with two different voltages, wherein at least one voltage is constant, maintained by the constant voltage circuitry.

Alternatively, when the energy received by the internal energy receiver is accumulated and stabilized in an energy stabilizing unit before energy is supplied to the medical device and/or energy storage device, the energy may be supplied to the medical device and/or energy storage device with at least one constant current, as maintained by a constant current circuitry. In that case, the medical device and energy storage device may be supplied with two different currents wherein at least one current is constant, maintained by the constant current circuitry.

The wireless energy may be initially transmitted according to a predetermined energy consumption plus storage rate. In that case, the transmission of wireless energy may be turned off when a predetermined total amount of energy has been transmitted. The energy received by the internal energy receiver may then also be accumulated and stabilized in an energy stabilizing unit before being consumed to operate the medical device and/or stored in the energy storage device until a predetermined total amount of energy has been consumed and/or stored.

Further, the wireless energy may be first transmitted with the predetermined energy rate, and then transmitted based on the energy balance which can be determined by detecting the total amount of accumulated energy in the energy stabilizing unit. Alternatively, the energy balance can be determined by detecting a change in the current amount of accumulated energy in the energy stabilizing unit. In yet another alternative, the energy balance, can be determined by detecting the direction and rate of change in the current amount of accumulated energy in the energy stabilizing unit.

The transmission of wireless energy may be controlled such that an energy reception rate in the internal energy receiver corresponds to the energy consumption and/or storage rate. In that case, the transmission of wireless energy may be turned off when a predetermined total amount of energy has been consumed.

The energy received by the internal energy receiver may be first accumulated and stabilized in an energy stabilizing unit, and then consumed or stored by the medical device until a predetermined total amount of energy has been consumed. In that case, the energy balance may be determined based on a detected total amount of accumulated energy in the energy stabilizing unit. Alternatively, the energy balance may be determined by detecting a change in the current amount of accumulated energy in the energy stabilizing unit. In yet another alternative, the energy balance may be determined by detecting the direction and rate of change in the current amount of accumulated energy in the energy stabilizing unit.

Suitable sensors may be used for measuring certain characteristics of the medical device and/or detecting the current condition of the patient, somehow relating to the amount of energy needed for proper operation of the medical device. Thus, electrical and/or physical parameters of the medical device and/or physical parameters of the patient may be determined, and the energy can then be transmitted with a transmission rate which is determined based on the parameters. Further, the transmission of wireless energy may be controlled such that the total amount of transmitted energy is based on said parameters.

The energy received by the internal energy receiver may be first accumulated and stabilized in an energy stabilizing unit, and then consumed until a predetermined total amount of energy has been consumed. The transmission of wireless energy may further be controlled such that an energy reception rate at the internal energy receiver corresponds to a predetermined energy consumption rate.

Further, electrical and/or physical parameters of the medical device and/or physical parameters of the patient may be determined, in order to determine the total amount of transmitted energy based on the parameters. In that case, the energy received by the internal energy receiver may be first accumulated and stabilized in an energy stabilizing unit, and then consumed until a predetermined total amount of energy has been consumed.

The energy is stored in the energy storage device according to a predetermined storing rate. The transmission of wireless energy may then be turned off when a predetermined total amount of energy has been stored. The transmission of wireless energy can be further controlled such that an energy reception rate at the internal energy receiver corresponds to the predetermined storing rate.

The energy storage device of the medical device may comprise a first storage device and a second storage device, wherein the energy received by the internal energy receiver is first stored in the first storage device, and the energy is then supplied from the first storage device to the second storage device at a later stage.

When using the first and second storage devices in the energy storage device, the energy balance may be determined in different ways. Firstly, the energy balance may be determined by detecting the current amount of energy stored in the first storage device, and the transmission of wireless energy may then be controlled such that a storing rate in the second storage device corresponds to an energy reception rate in the internal energy receiver. Secondly, the energy balance may be determined based on a detected total amount of stored energy in the first storage device. Thirdly, the energy balance may be determined by detecting a change in the current amount of stored energy in the first storage device. Fourthly, the energy balance may be determined by detecting the direction and rate of change in the current amount of stored energy in the first storage device.

Stabilized energy may be first supplied from the first storage device to the second storage device with a constant current, as maintained by a constant current circuitry, until a measured voltage over the second storage device reaches a predetermined maximum voltage, and thereafter supplied from the first storage device to the second storage energy storage device with a constant voltage, as maintained by a constant voltage circuitry. In that case, the transmission of wireless energy may be turned off when a predetermined minimum rate of transmitted energy has been reached.

The transmission of energy may further be controlled such that the amount of energy received by the internal energy receiver corresponds to the amount of energy stored in the second storage device. In that case, the transmission of energy may be controlled such that an energy reception rate at the internal energy receiver corresponds to an energy storing rate in the second storage device. The transmission of energy may also be controlled such that a total amount of received energy at the internal energy receiver corresponds to a total amount of stored energy in the second storage device.

In the case when the transmission of wireless energy is turned off when a predetermined total amount of energy has been stored, electrical and/or physical parameters of the medical device and/or physical parameters of the patient may be determined during a first energy storing procedure, and the predetermined total amount of energy may be stored in a subsequent energy storing procedure based on the parameters.

When electrical and/or physical parameters of the medical device and/or physical parameters of the patient are determined, the energy may be stored in the energy storage device with a storing rate which is determined based on the parameters. In that case, a total amount of energy may be stored in the energy storage device, the total amount of energy being determined based on the parameters. The transmission of wireless energy may then be automatically turned off when the total amount of energy has been stored. The transmission of wireless energy may further be controlled such that an energy reception rate at the internal energy receiver corresponds to the storing rate.

When electrical and/or physical parameters of the medical device and/or physical parameters of the patient are determined, a total amount of energy may be stored in the energy storage device, the total amount of energy being determined based on said parameters. The transmission of energy may then be controlled such that the total amount of received energy at the internal energy receiver corresponds to the total amount of stored energy. Further, the transmission of wireless energy may be automatically turned off when the total amount of energy has been stored.

When the energy used for the medical device is partly consumed and partly stored, the transmission of wireless energy may be controlled based on a predetermined energy consumption rate and a predetermined energy storing rate. In that case, the transmission of energy may be turned off when a predetermined total amount of energy has been received for consumption and storage. The transmission of energy may also be turned off when a predetermined total amount of energy has been received for consumption and storage.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When electrical and/or physical parameters of the medical device and/or physical parameters of the patient are determined, the energy may be supplied from the energy storage device to the medical device for consumption with a supply rate which is determined based on said parameters. In that case, the total amount of energy supplied from the energy storage device to the medical device for consumption, may be based on said parameters.

When electrical and/or physical parameters of the medical device and/or physical parameters of the patient are determined, a total amount of energy may be supplied to the medical device for consumption from the energy storage device, where the total amount of supplied energy is determined based on the parameters.

When the energy received by the internal energy receiver is accumulated and stabilized in an energy stabilizing unit, the energy balance may be determined based on an accumulation rate in the energy stabilizing unit, such that a storing rate in the energy storage device corresponds to an energy reception rate in the internal energy receiver.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

When using the first and second storage devices in the energy storage device, the second storage device may directly or indirectly supply energy to the medical device, wherein the change of the difference corresponds to a change of the amount of energy accumulated in the first storage unit. The energy balance may then be determined by detecting a change over time in the energy storing rate in the first storage device, the energy balance corresponding to the change. The change in the amount of stored energy may also be detected by determining overtime the derivative of a measured electrical parameter indicating the amount of stored energy, the derivative corresponding to the change in the amount of stored energy. A rate of change of the electrical parameter may also be detected, the derivative being related to the change rate. The electrical parameter may be a measured voltage and/or current related to the energy balance.

The first storage device may include at least one of: a capacitor and a semiconductor, and the second storage device includes at least one of: a rechargeable battery, an accumulator and a capacitor.

The wireless energy may be transmitted in pulses or waves and/or by means of an electric field and may then be controlled by adjusting the width of the pulses.

When the difference between the total amount of energy received by the internal energy receiver and the total amount of consumed energy is measured over time, directly or indirectly, the energy balance may be determined by detecting a change in the difference. In that case, the change in the amount of consumed energy may be detected by determining over time the derivative of a measured electrical parameter related to the amount of consumed energy, the derivative corresponding to the rate of the change in the amount of consumed energy, wherein the rate of change includes the direction and speed of the change. A rate of change of the electrical parameter may then be detected, the derivative being related to the detected change rate.

When using the first and second storage devices in the energy storage device, the first storage device may be adapted to be charged at a relatively higher energy charging rate as compared to the second storage device, thereby enabling a relatively faster charging. The first storage device may also be adapted to be charged at multiple individual charging occasions more frequently as compared to the second storage device, thereby providing relatively greater life-time in terms of charging occasions. The first storage device may comprise at least one capacitor. Normally, only the first storage may be charged and more often than needed for the second storage device.

When the second storage device needs to be charged, to reduce the time needed for charging, the first storage device is charged at multiple individual charging occasions, thereby leaving time in between the charging occasions for the first storage device to charge the second storage device at a relatively lower energy charging rate. When electrical parameters of the medical device are determined, the charging of the second storage device may be controlled based on the parameters. A constant current or stabilizing voltage circuitry may be used for storing energy in the second storage device.

### Embodiments to Control the Wireless Energy Supply Based on the Feed Back Mechanism Above

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

Thus, wireless energy is transmitted from an external energy transmitting device placed externally to a human body to an internal energy receiver placed internally in the human body. Electrical pulses from a first electric circuit may be applied to the external transmitting device to transmit the wireless energy, the electrical pulses having leading and trailing edges. the lengths of first time intervals between successive leading and trailing edges of the electrical pulses and/or the lengths of second time intervals between successive trailing and leading edges of the electrical pulses, may be varied. The transmitted energy generated from the electrical pulses may further have a varied power, the varying of the power depending on the lengths of the first and/or second time intervals.

An apparatus adapted to transmit wireless energy from an external energy transmitting device placed externally to a human body to an internal energy receiver placed internally in the human body, may comprise a first electric circuit to supply electrical pulses to the external transmitting device, said electrical pulses having leading and trailing edges, said transmitting device adapted to supply wireless energy, wherein the electrical circuit is adapted to vary the lengths of first time intervals between successive leading and trailing edges of the electrical pulses and/or the lengths of second time intervals between successive trailing and leading edges of the electrical pulses, and wherein the transmitted wireless energy, generated from the electrical pulses having a varied power, the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied.

When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

When applying electrical pulses to the external energy source, the electrical pulses may generate an electromagnetic field over the external energy source, the electromagnetic field being varied by varying the first and second time intervals, and the electromagnetic field may induce electrical pulses in the internal energy receiver, the induced pulses carrying energy transmitted to the internal energy receiver.

The electrical pulses may be released from the first electrical circuit with such a frequency and/or time period between leading edges of the consecutive pulses, so that when the lengths of the first and/or second time intervals are varied, the resulting transmitted energy are varied. When applying the electrical pulses, the electrical pulses may have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

### T: Constriction of a Luminary Organ

The wireless energy may be used for controlling a flow of fluid and/or other bodily matter in a lumen formed by a tissue wall of a patient's organ. At least one portion of the tissue wall may then be gently constricted to influence the flow in the lumen, and the constricted wall portion may be stimulated to cause contraction of the wall portion to further influence the flow in the lumen.

The object of the present embodiment is to provide an apparatus adapted to control or a method for controlling the flow of fluids and/or other bodily matter in lumens formed by tissue walls of bodily organs, so as to at least substantially or even completely eliminate the injured tissue wall problems that have resulted from implanted prior art devices that constrict such bodily organs.

In accordance with this object of the present embodiment, there is provided an apparatus adapted to control or a method for controlling the flow of fluids and/or other bodily matter in a lumen that is formed by the tissue wall of a bodily organ, the apparatus comprising an implantable constriction device for gently constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.

The embodiments above may provide an advantageous combination of constriction and stimulation devices, which results in a two-stage influence on the flow of fluids and/or other bodily matter in the lumen of a bodily organ. Thus, the constriction device may gently constrict the tissue wall by applying a relatively weak force against the wall portion, and the stimulation device may stimulate the constricted wall portion to achieve the desired final influence on the flow in the lumen. The phrase “gently constricting a portion of the tissue wall” is to be understood as constricting the wall portion without substantially hampering the blood circulation in the tissue wall.

Thus both a method for controlling the flow in the lumen and an apparatus adapted to control the flow in the lumen may be implemented according to different embodiments and features as follows: Preferably, the stimulation device is adapted to stimulate different areas of the wall portion as the constriction device constricts the wall portion, and the control device controls the stimulation device to intermittently and individually stimulate the areas of the wall portion. This intermittent and individual stimulation of different areas of the wall portion of the organ allows tissue of the wall portion to maintain substantially normal blood circulation during the operation of the apparatus above.

The combination of the constriction and stimulation devices enables application of the apparatus or method above at any place on any kind of bodily organs, in particular, but not limited to, tubular bodily organs, which is a significant advance in the art, as compared with prior stimulation devices that are confined to electric stimulation of malfunctioning sphincters.

In some applications, there will be daily adjustments of the implanted constriction device. Therefore, the constriction device may be adjustable to enable adjustment of the constriction of the wall portion as desired, wherein the control device controls the constriction device to adjust the constriction of the wall portion. The control device may control the constriction and stimulation devices independently of each other, and simultaneously. Optionally, the control device may control the stimulation device to stimulate, or to not stimulate the wall portion while the control device controls the constriction device to change the constriction of the wall portion.

Initially, the constriction device may be calibrated by using the control device to control the stimulation device to stimulate the wall portion, while controlling the constriction device to adjust the constriction of the wall portion until the desired restriction of the flow in the lumen is obtained.

### Flow Restriction

The apparatus may be used for restricting the flow of fluids and/or other bodily matter in the lumen of a bodily organ. Thus, in one embodiment, the constriction device is adapted to constrict the wall portion to at least restrict the flow in the lumen, and the control device controls the stimulation device to cause contraction of the constricted wall portion, so that the flow in the lumen is at least further restricted. Specifically, the constriction device is adapted to constrict the wall portion to a constricted state in which the blood circulation in the constricted wall portion is substantially unrestricted and the flow in the lumen is at least restricted, and the control device controls the stimulation device to cause contraction of the wall portion, so that the flow in the lumen is at least further restricted when the wall portion is kept by the constriction device in the constricted state.

The constriction and stimulation devices may be controlled to constrict and stimulate, respectively, to an extent that depends on the flow restriction that is desired to be achieved in a specific application of the apparatus above. Thus, in accordance with a first flow restriction option, the control device controls the constriction device to constrict the wall portion, such that flow in the lumen is restricted or stopped, and controls the stimulation device to stimulate the constricted wall portion to cause contraction thereof, such that flow in the lumen is further restricted or more safely stopped. More precisely, the control device may control the stimulation device in a first mode to stimulate the constricted wall portion to further restrict or stop the flow in the lumen and to:
a) control the stimulation device in a second mode to cease the stimulation of the wall portion to increase the flow in the lumen; or
b) control the stimulation and constriction devices in the second mode to cease the stimulation of the wall portion and release the wall portion to restore the flow in the lumen.
   Movement of Fluid and/or Other Bodily Matter in Lumen

In one embodiment the constriction device is adapted to constrict the wall portion to restrict or vary the flow in the lumen, and the control device controls the stimulation device to progressively stimulate the constricted wall portion, in the downstream or upstream direction of the lumen, to cause progressive contraction of the wall portion to move the fluid and/or other bodily matter in the lumen.

### Stimulation

The control device may control the stimulation device to stimulate one or more of the areas of the wall portion at a time, for example by sequentially stimulating the different areas. Furthermore, the control device may control the stimulation device to cyclically propagate the stimulation of the areas along the wall portion, preferably in accordance with a determined stimulation pattern. To achieve the desired reaction of the tissue wall during the stimulation thereof, the control device may control the stimulation device to, preferably cyclically, vary the intensity of the stimulation of the wall portion.

In another embodiment, the control device controls the stimulation device to intermittently stimulate the areas of the wall portion with pulses that preferably form pulse trains. At least a first area and a second area of the areas of the wall portion may be repeatedly stimulated with a first pulse train and a second pulse train, respectively, such that the first and second pulse trains over time are shifted relative to each other. For example, the first area may be stimulated with the first pulse train, while the second area is not stimulated with said second pulse train, and vice versa. Alternatively, the first and second pulse trains may be shifted relative to each other, such that the first and second pulse trains at least partially overlap each other.

The pulse trains can be configured in many different ways. Thus, the control device may control the stimulation device to vary the amplitudes of the pulses of the pulse trains, the duty cycle of the individual pulses of each pulse train, the width of each pulse of the pulse trains, the length of each pulse train, the repetition frequency of the pulses of the pulse trains, the repetition frequency of the pulse trains, the number of pulses of each pulse train, and/or the off-time periods between the pulse trains. Several pulse trains of different configurations may be employed to achieve the desired effect.

In case the control device controls the stimulation device to vary the off-time periods between pulse trains that stimulate the respective area of the wall portion, it is also possible to control each off-time period between pulse trains to last long enough to restore substantially normal blood circulation in the area when the latter is not stimulated during the off-time periods.

An electric stimulation device suitably comprises at least one, preferably a plurality of electrical elements, such as electrodes, for engaging and stimulating the wall portion with electric pulses. Optionally, the electrical elements may be placed in a fixed orientation relative to one another. The control device controls the electric stimulation device to electrically energize the electrical elements, one at a time, or groups of electrical elements at a time. Preferably, the control device controls the electric stimulation device to cyclically energize each element with electric pulses. Optionally, the control device may control the stimulation device to energize the electrical elements, such that the electrical elements are energized one at a time in sequence, or such that a number or groups of the electrical elements are energized at the same time. Also, groups of electrical elements may be sequentially energized, either randomly or in accordance with a predetermined pattern.

The electrical elements may form any pattern of electrical elements. Preferably, the electrical elements form an elongate pattern of electrical elements, wherein the electrical elements are applicable on the patient's wall of the organ, such that the elongate pattern of electrical elements extends lengthwise along the wall of the organ, and the elements abut the respective areas of the wall portion. The elongate pattern of electrical elements may include one or more rows of electrical elements extending lengthwise along the wall of the organ. Each row of electrical elements may form a straight, helical, or zig-zag path of electrical elements, or any form of path. The control device may control the stimulation device to successively energize the electrical elements longitudinally along the elongate pattern of electrical elements in a direction opposite to, or in the same direction as that of, the flow in the patient's lumen.

In accordance with one embodiment, the electrical elements form a plurality of groups of elements, wherein the groups form a series of groups extending along the patient's organ in the flow direction in the patient's lumen. The electrical elements of each group of electrical elements may form a path of elements extending at least in part around the patient's organ. In a first alternative, the electrical elements of each group of electrical elements may form more than two paths of elements extending on different sides of the patient's organ, preferably substantially transverse to the flow direction in the patient's lumen. The control device may control the stimulation device to energize the groups of electrical elements in the series of groups in random, or in accordance with a predetermined pattern. Alternatively, the control device may control the stimulation device to successively energize the groups of electrical elements in the series of groups in a direction opposite to, or in the same direction as that of, the flow in the patient's lumen, or in both said directions starting from a position substantially at the center of the constricted wall portion. For example, groups of energized electrical elements may form advancing waves of energized electrical elements, as described above; that is, the control device may control the stimulation device to energize the groups of electrical elements, such that energized electrical elements form two waves of energized electrical elements that simultaneously advance from the center of the constricted wall portion in two opposite directions towards both ends of the elongate pattern of electrical elements.

### Mechanical Operation

Where the operation device mechanically operates the constriction device of the constriction/stimulation unit, it may be non-inflatable. Furthermore, the operation device may comprise a servo system, which may include a gearbox. The term “servo system” encompasses the normal definition of a servo mechanism, i.e., an automatic device that controls large amounts of power by means of very small amounts of power but may alternatively or additionally encompass the definition of a mechanism that transfers a weak force acting on a moving element having a long stroke into a strong force acting on another moving element having a short stroke. Preferably, the operation device operates the constriction device in a non-magnetic and/or non-manual manner. A motor may be operatively connected to the operation device. The operation device may be operable to perform at least one reversible function and the motor may be capable of reversing the function.

### Hydraulic Operation

Where the operation device hydraulically operates the constriction device of the constriction/stimulation unit, it includes hydraulic means for adjusting the constriction device.

In another embodiment, the hydraulic means comprises a reservoir and an expandable/contractible cavity in the constriction device, wherein the operation device distributes hydraulic fluid from the reservoir to expand the cavity and distributes hydraulic fluid from the cavity to the reservoir to contract the cavity. The cavity may be defined by a balloon of the constriction device that abuts the tissue wall portion of the patient's organ, so that the patient's wall portion is constricted upon expansion of the cavity and released upon contraction of the cavity.

Alternatively, the cavity may be defined by a bellows that displaces a relatively large contraction element of the constriction device, for example a large balloon that abuts the wall portion, so that the patient's wall portion is constricted upon contraction of the bellows and released upon expansion of the bellows. Thus, a relatively small addition of hydraulic fluid to the bellows causes a relatively large increase in the constriction of the wall portion. Such a bellows may also be replaced by a suitably designed piston/cylinder mechanism.

Where the hydraulic means comprises a cavity in the constriction device, the apparatus above can be designed in accordance with the options listed below.
1) The reservoir comprises first and second wall portions, and the operation device displaces the first and second wall portions relative to each other to change the volume of the reservoir, such that fluid is distributed from the reservoir to the cavity, or from the cavity to the reservoir.
   1a) The first and second wall portions of the reservoir are displaceable relative to each other by at least one of a magnetic device, a hydraulic device, or an electric control device. means.
2) The apparatus comprises a fluid conduit between the reservoir and the cavity, wherein the reservoir forms part of the conduit. The conduit and reservoir and apparatus are devoid of any non-return valve. The reservoir forms a fluid chamber with a variable volume and distributes fluid from the chamber to the cavity by a reduction in the volume of the chamber and withdraws fluid from the cavity by an expansion of the volume of the chamber. The apparatus further comprises a motor for driving the reservoir, comprising a movable wall of the reservoir for changing the volume of the chamber.

In another embodiment, the operation device comprises a reverse servo operatively connected to the hydraulic means. The term “reverse servo” is to be understood as a mechanism that transfers a strong force acting on a moving element having a short stroke into a weak force acting on another moving element having a long stroke; i.e., the reverse function of a normal servo mechanism. Thus, minor changes in the amount of fluid in a smaller reservoir could be transferred by the reverse servo into major changes in the amount of fluid in a larger reservoir. The reverse servo is particularly suited for manual operation thereof.

### Design of Control Device

The control device suitably controls the constriction/stimulation unit from outside the patient's body. Preferably, the control device is operable by the patient. For example, the control device may comprise a manually operable switch for switching on and off the constriction/stimulation unit, wherein the switch is adapted for subcutaneous implantation in the patient to be manually or magnetically operated from outside the patient's body. Alternatively, the control device may comprise an external control unit in the form of a hand-held wireless remote control, which is conveniently operable by the patient to switch on and off the constriction/stimulation unit. The wireless remote control may also be designed for application on the patient's body like a wristwatch. Such a wristwatch type of remote control may emit a control signal or the like that follows the patient's body to implanted signal responsive means of the apparatus.

The transmission of wireless energy from the external energy transmitting device may be controlled by applying to the external energy transmitting device electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

Thus, it is provided a method of controlling transmission of wireless energy, and the method may further comprise:
applying to the external transmitting device electrical pulses from a first electric circuit to transmit the wireless energy, the electrical pulses having leading and trailing edges,
varying the lengths of first-time intervals between successive leading and trailing edges of the electrical pulses and/or the lengths of second time intervals between successive trailing and leading edges of the electrical pulses, and
transmitting wireless energy, the transmitted energy generated from the electrical pulses having a varied power, the varying of the power depending on the lengths of the first and/or second time intervals.

Also is provided an apparatus adapted to transmit wireless energy from an external energy transmitting device placed externally to a human body to an internal energy receiver placed internally in the human body. The apparatus may comprise,
a first electric circuit to supply electrical pulses to the external transmitting device, said electrical pulses having leading and trailing edges, said transmitting device adapted to supply wireless energy, wherein
the electrical circuit being adapted to vary the lengths of first-time intervals between successive leading and trailing edges of the electrical pulses and/or the lengths of second time intervals between successive trailing and leading edges of the electrical pulses, and wherein
the transmitted wireless energy, generated from the electrical pulses having a varied power, the power depending on the lengths of the first and/or second time intervals.

As stated above, there is a need for a solution by means of which energy can be supplied inductively to an implanted medical device from an external energy source, i.e. external to the patient's body, and by means of which the transfer of energy can be adjusted to the needs of the medical device in a manner which is more rapid than is possible by means of known such systems.

Such a solution is offered by the present invention in that it discloses a medical system which comprises internal parts for implantation in a patient and external parts for use externally to the patient.

The external parts comprise an energy source which is equipped with a primary coil for inductively transmitting energy to the implantable parts and a control unit for the control of the energy source. The internal parts comprise an electrically powered medical device, an energy receiver equipped with a secondary coil for inductively receiving energy for the medical device from the external energy source and a control unit for the control of the internal parts.

The inventive system is arranged to determine a balance between the amount of energy received in the energy receiver and the amount of energy used by the medical device, and the internal control unit is arranged to wirelessly transmit feedback information to the external control unit.

According to the invention, the system is arranged to determine the feedback information based on or relating to a first and a second parameter. The first parameter is based on the previously mentioned energy balance seen over a certain amount of time, and the second parameter is based on information determined by the system and relating to a coupling factor between the primary and the secondary coil.

The system is adapted to take into account at least both the first and second parameters in order to determine the amount of energy which should be transmitted by the external energy source, thus allowing for a rapid adjustment of said energy balance.

In one preferred embodiment the medical system comprises internal parts for implantation in a patient and external parts for use externally to the patient, the external parts comprising an energy source equipped with a primary coil for inductively transmitting energy to the implantable parts and an external control unit for the control of the energy source, the internal parts comprising an electrically powered medical device, an energy receiver equipped with a secondary coil for inductively receiving energy for the medical device from the external energy source and an internal control unit for the control of the internal parts, the system being arranged to determine a balance between the amount of energy received in the energy receiver and the amount of energy used by the medical device, in which system the internal control unit is arranged to wirelessly transmit feedback information to the external control unit, the system being characterized in that it is arranged to determine said feedback information based on or relating to a first and a second parameter and a third parameter, with the first parameter being based on said energy balance over a certain amount of time and the second parameter being based on information relating to a coupling factor between the primary and the secondary coils and the third parameter being based on at least one of;
a) in which the medical device also comprises a current regulator arranged to keep a current constant, the system being arranged to determine a difference between an input current to the current regulator and the current which the current regulator is arranged to keep constant, in which system said third parameter comprising or being based on said difference,
b) in which the system also comprises a voltage regulator arranged to keep a voltage constant, the system being arranged to determine a difference between an input voltage to the voltage regulator and the voltage which the voltage regulator is arranged to keep constant, in which system said third parameter comprising or being based on said difference, and wherein
the system being adapted to take into account at least both the first and second and third parameters to determine the amount of energy which should be transmitted by the energy source, thus allowing for a rapid adjustment of said energy balance.

It is important to understand the difference between the calibration of the system and the continuous regular feed-back. The platform for the control of the energy transfer is the energy balance between the received and used energy by the implant (the used energy includes energy stored by the implant). However, to fast get an accurate level of energy transfer the transmission is initially calibrated with the coupling factor, basically the relation between the externally transmitted energy and the internally received energy. Furthermore, the coupling factor is followed over time to see if any new recalibration is needed, for example if the outer transmitting coil is moved in relation to the inner receiving coil. A rapid movement of the outer coil by for example an action by the patient could cause such a large change of the amount of received energy that the regular feedback normally taking small steps is not fast enough. Therefore a new calibration of the coupling factor could in one fast step recalibrate the energy transmission level. Furthermore, because the implant could not tolerate to get hot it is extremely important to use all energy very efficient. Normally the implant needs one or more fixed voltages or maybe also fixed current levels to work properly. When creating a fixed voltage level the voltage regulator needs a certain voltage span, that's to say a higher input voltage to the regulator than the fixed output voltage the regulator delivers. On the other hand if this difference between the input voltage and the fixed output voltage gets larger than needed the extra voltage will be consumed and still the regulator will deliver the requested output voltage although creating more heat. It is therefore of outmost importance to also keep track of any voltage or current difference over any regulator in the implanted implant to optimize energy consumption and heat production.

Additionally the system may also comprises a capacitor coupled in parallel over the medical device, the system being arranged to determine the total amount of energy stored in said capacitor, in which system the feedback information is also based on or comprises a parameter comprising or being based on the total amount of energy stored in the capacitor, the system also being adapted to take into this parameter in order to determine the amount of energy which should be transmitted by the external energy source.

In a further additional embodiment the system is adapted to use all at least all three parameters to determine the feedback information, wherein the feedback information comprises information comprising or relating to the amount of energy which should be transmitted by the external energy source.

Preferably the system is adapted to use the second and third parameters for determining the amount of energy which should be transmitted by the external energy source as a repeated calibration of the system, and to use the first parameter during operation of the system as a regular feedback in order to determine the amount of energy which should be transmitted by the external energy source during operation of the system.

In one embodiment of the system of the invention, the medical device also comprises a current regulator arranged to keep a current constant, and the system is arranged to determine a difference between an input current to the current regulator and the current which the current regulator is arranged to keep constant. In such a system, the feedback information is also based on or comprises a third parameter which comprises or is based on the current difference, and the system is adapted to also take into account the third parameter in order to determine the amount of energy which should transmitted by the external energy source.

In one embodiment of the system of the invention, the medical device also comprises a voltage regulator arranged to keep a voltage constant and the system is arranged to determine a difference between an input voltage to the voltage regulator and the voltage which the voltage regulator is arranged to keep constant. In such a system, the feedback information is also based on or comprises a third parameter which comprises or is based on the voltage difference, and the system is adapted to also take into account this third parameter in order to determine the amount of energy which should transmitted by the external energy source.

In one embodiment, the system is adapted to use all three parameters to determine the feedback information, and the feedback information comprises information comprising or relating to the amount of energy which should be transmitted by the external energy source.

In one embodiment, the system is adapted to use the second and third parameters for determining the amount of energy which should be transmitted by the external energy source, and to use the first parameter during operation of the system in order to determine the amount of energy which should be transmitted by the external energy source during operation of the system.

In yet one embodiment the system, the energy receiver comprises a first switch adapted to switch a connection between the secondary coil and the medical device on and off. The connection between the secondary coil and the medical device is switched on and off in order to achieve at least one of the following; a) enable the system to measure the coupling factor when the connection is off and b) using said switch as a security system, switching off when a security parameter determined by the internal control unit relating to the wireless energy transfer exceeds a certain threshold.

Suitably, the external control unit is adapted to transmit information wirelessly to the internal control unit which in turn is adapted to receive information wirelessly.

As will be realized, when it comes to determining the feedback parameters, this task can be divided between the internal and the external units (suitably their respective control units) in a rather large number of ways within the scope of the invention. In one embodiment, the external control unit can supply the internal control unit with information necessary to determine the second parameter, and the internal control unit can be given the task of determining all of the parameters as such, and to then supply them to the external control unit as feedback information. In such an embodiment, it is sufficient if the internal control unit supplies the external control unit with a percentage figure for a variation of the energy supply as the feedback information. Naturally, the percentage can be positive, negative or zero, in order to indicate an increase, a decrease, or a maintained energy transfer level.

In another embodiment, the internal control unit supplies the external control unit with information which is sufficient for the external control unit to establish the parameters which are used by the system, with that information then being the feedback information.

Again, as will be realized, the task of determining the feedback parameters can be divided in a large number of ways between the internal and external units within the scope of the invention, which will also impact on the nature and contents of the feedback information.

Thus, in one embodiment, at least one of the parameters is transmitted from the external control unit to the internal control unit, and the internal control unit determines the other parameters used by the system and transmits the feedback information to the external control unit as information on the amount of energy which should transmitted by the external energy source.

In one embodiment, information for determining at least one of the parameters is transmitted from the external control unit to the internal control unit, and the internal control unit determines the parameters used by the system and transmits the feedback information to the external control unit as information on the amount of energy which should transmitted by the external energy source.

In one embodiment, information for determining at least one of the parameters is transmitted as the feedback information from the internal control unit to the external control unit, and the external control unit determines the parameters used by the system as well as the amount of energy which should transmitted by the external energy source.

In one embodiment, at least one of the parameters is transmitted as the feedback information from the internal control unit to the external control unit, and the external control unit determines the other parameters used by the system as well as the amount of energy which should transmitted by the external energy source.

The various aforementioned features of the embodiments may be combined in any way if such combination is not clearly contradictory. Embodiments will now be described in more detail in reference to the accompanying drawings. Again, individual features of the various embodiments may be combined or exchanged unless such combination or exchange is clearly contradictory to the overall function of the device.

### U: Stretch

Apparatuses and devices for treating obesity are provided. In particular these apparatuses and devices comprise a stretching device.

An apparatus for treating obesity of a patient having a stomach with a food cavity is provided, the apparatus comprising:—a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein at least one adjustable stretching device adapted to be at least substantially invaginated by a stomach wall portion of the patient with the outer surface of the stretching device resting against the stomach wall and adapted to stretch a portion of stomach wall, and—a fluid connection device interconnecting the volume filling device and the stretching device.

A device for treating obesity of a patient is provided, the device comprising: at least one operable stretching device implantable in the patient and adapted to stretch a portion of the patient's stomach wall, and an implantable control unit for automatically controlling the operable stretching device, when the control unit and stretching device are implanted, to stretch the stomach wall portion in connection with the patient eating such that satiety is created, wherein the stretching device comprises a first and a second engaging part, wherein: the first part is adapted to be engaged to a first area of the stomach wall, and the second part is adapted to be engaged to a second area of the stomach wall, and wherein the stretching device is adapted to stretch a portion of the stomach wall between the first area and the second area.

An obesity treatment device is provided, comprising:—at least one operable stretching device implantable in a patient and adapted to stretch a portion of the patient's stomach wall and—an implantable operation device for operating the stretching device, when implanted, to stretch the stomach wall portion such that satiety is created, wherein the operable stretching device is adapted to be placed against the outside of the stomach wall.

A device for treating obesity of a patient is provided, the device comprising: at least one operable stretching device implantable in the patient and adapted to stretch a portion of the patient's stomach wall, and an implantable control unit for controlling the operable stretching device, when the control unit and the stretching device are implanted, to stretch the stomach wall portion in connection with the patient eating such that satiety is created, wherein the stretching device comprises a first engaging part and a second engaging part, wherein: the first engaging part is adapted to be engaged to a first area of the stomach wall, and the second engaging part is adapted to be engaged to a second area of the stomach wall, and wherein the stretching device is adapted to stretch a portion of the stomach wall between the first area and the second area.

A device for treating obesity of a patient is provided, the device comprising: a. at least one operable stretching device implantable in the patient operating via mechanical, pneumatic, and/or hydraulic action to stretch a portion of the patient's stomach wall, and b. an implantable control unit for automatically controlling the operable stretching device, when the control unit and stretching device are implanted, to stretch the stomach wall portion in connection with the patient eating such that satiety is created.

### V: Anal Incontinence Treatment

According to an aspect, a new convenient and reliable anal incontinence treatment apparatus, having a long lifetime, is provided. In particular, the patient should be able to control the apparatus at any time after the operation when various needs arise over the course of a day, so that the patient substantially always is satisfied or comfortable. Accordingly, the present invention provides an anal incontinence treatment apparatus which comprises: An energy transmission means for wireless transmission of energy of a first form from outside a patient's body. An operable restriction device implantable in the patient for engaging the rectum or colon to form a restricted fecal passageway in the rectum or colon, the device operable in response to energy of a second form different than the energy of the first form to vary the restricted passageway. And, an energy transforming means implantable in the patient for transforming energy of the first form wirelessly transmitted by the energy transmission means into energy of the second form.

As a result, the advantage is achieved that the anal incontinence treatment apparatus provides simple and effective energy transmission which ensures an extended and reliable functionality of the apparatus, possibly for the rest of the patient's natural life, and at least many years. Preferably, the energy transforming means comprises at least one element having a positive region and a negative region, and adapted to create an energy field between the positive and negative regions when exposed to the energy of the first form transmitted by the energy transmission means, so that the energy field produces the energy of the second form. Alternatively, at least one semiconductor circuitry, transistor circuitry or microchip may be substituted for the element having a positive and a negative region. Such a semiconductor circuitry, transistor circuitry or microchip is adapted to create an energy field when exposed to the energy of the first form wirelessly transmitted by the energy transmission means, whereby the energy field provides the energy of the second form.

The restriction device preferably controls the cross-sectional area of the fecal passageway in the rectum or colon, which gives the advantage that the patient is enabled to adjust the cross-sectional area of the fecal passageway whenever he likes during the day. This advantage should not be underestimated.

Advantageously, the restriction device is directly operated with the energy of the second form, preferably in a non-magnetic and/or non-mechanical manner, as the energy transmission means transmits the energy of the first form. The restriction device may be directly operated with the energy of the second form without externally touching subcutaneously implanted components of the apparatus. The advantage of directly using energy as it is transmitted is that the apparatus can be of a very simple design and the few components involved makes the apparatus extremely reliable.

The restriction device may be non-inflatable, i.e., with no hydraulic or pneumatic fluid involved for the adjustments of the restriction device. This eliminates problems with fluid leaking from the restriction device.

In accordance with a preferred embodiment of the invention, the element comprises an electrical junction element capable of inducing an electric field between the positive and negative regions when exposed to the energy of the first form transmitted by the energy transmission means, whereby the energy of the second form comprises electric energy.

Consequently, the restriction device suitably is electrically operated, whereby the positive and negative regions of the electrical junction element supply electric energy for the operation of the restriction device. The apparatus suitably comprises implantable electric conductors connected to the positive and negative regions of the electrical junction element, whereby the electrical junction element is capable of supplying an electric current, such as a direct current, a pulsating direct current, a combination of a direct and pulsating direct current, an alternating current or a combination of a direct and alternating current, via the conductors. Furthermore, the electrical junction element may be capable of supplying a frequency, amplitude, or frequency and amplitude modulated analog, digital, or a combination of analog and digital signal, which is used in connection with control of the restriction device.

The element, preferably in the form of an electrical semiconductor junction element, suitably forms a flat and thin sheet and has a volume of less than 2000 cm3 to be suited for subcutaneous implantation, so that the electrical junction element can be located just behind the skin of the patient. The electrical junction element should be designed to generate an output current exceeding 1 μA when exposed to the energy of the first form transmitted by the energy transmission means. Of course, all the components of the energy transforming means including the electrical junction element in contact with the patient's body should be of a biocompatible material. Alternatively, it would be possible to implant the energy transforming means in the thorax or cephal region of the patient, or in an orifice of the patient's body and under the mucosa or intraluminar outside the mucosa of the orifice.

For in vitro appliances, a particular type of an electrical semiconductor junction element has been commonly used, namely a so-called p-n (positive/negative) junction element, typically in the form of solar cells. A solar cell transfers solar energy in the form of visible light into electric energy in the form of direct current. For example, a p-n junction element may comprise two layers of semiconductor, one p-type (positive) and the other n-type (negative), sandwiched together to form a “p-n junction”. This p-n junction induces an electric field across the element when absorbing quanta of light (photons). To be more precise, the quanta of light transfer their energy to some of the semiconductor's electrons, which are then able to move about through the material. For each such negatively charged electron, a corresponding positive charge—a “hole”—is created. In an ordinary semiconductor, these electrons and holes recombine after a short time and their energy is wasted as heat. However, when the electrons and holes are swept across the p-n junction in opposite directions by the action of the electric field, the separation of charge induces a voltage across the p-n junction element. By connecting the p-n junction element to an external circuit, the electrons are able to flow thereby creating a current.

Surprisingly, it has been proved that although both the skin and subcutis absorb energy from an external light beam directed against the skin portion behind which a properly designed p-n junction element is located, the light energy transmitted through the skin can induce a current from the p-n junction element strong enough (minimum 1 μA) to enable the operation of the electrically operated restriction device. Thus, such a p-n junction element is now for the first time used for in vivo applications.

However, the apparatus of the present invention is not limited to the use of visible light for the wireless transmission of energy. Thus, in accordance with a broad aspect of the invention, the energy transmission means transmits energy by at least one wireless signal, preferably containing radiant energy.

The wireless signal may comprise a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined.

Alternatively, the wave signal may comprise a sound wave signal, such as an ultrasonic signal. Generally, the wireless signal may comprise a digital, analog or a digital and analog signal. The energy of the first form transmitted by the energy transmission means may comprise an electric or magnetic field transmitted in pulses, for example digital pulses. Furthermore, the energy transforming means may transfer the energy of the first form, which may comprise polarized energy, into a direct current, pulsating direct current, a combination of a direct and pulsating direct current, an alternating current or a combination of a direct and alternating current. Alternatively, the energy of the first form may comprise kinetic energy.

The energy of the second form may comprise a frequency, amplitude or frequency and amplitude modulated analog, digital or combined analog and digital signal.

The apparatus may further comprise an implantable pulse generator for generating electrical pulses from the energy of the second form rendered by the energy field created by the element having positive and negative regions.

In accordance with another embodiment of the invention, the apparatus comprises an implantable operation device for operating the restriction device and a control device for controlling the operation device, wherein the element powers the operation device with the energy of the second form. The operation device preferably comprises a motor, for example an electric linear motor or an electric rotary motor which is controlled by the control device to rotate a desired number of revolutions. The electric motor may have electrically conductive parts made of plastics. Alternatively, the motor may comprise a hydraulic or pneumatic fluid motor, wherein the control device controls the fluid flow through the fluid motor. Motors currently available on the market are getting smaller and smaller. Furthermore, there is a great variety of control methods and miniaturized control equipment available. For example, a number of revolutions of a rotary motor may be analyzed by a Hall-element just a few mm in size.

In accordance with another embodiment of the invention, the restriction device comprises hydraulic means and the operation device comprises a pump for pumping a fluid in the hydraulic means, a motor for driving the pump, a valveless fluid conduit between the pump and the hydraulic means of the restriction device, and a reservoir for fluid, wherein the reservoir forms part of the conduit. All of the hydraulic components involved are preferably devoid of any non-return valve. This is of great advantage, because with valves involved there is always a risk of malfunction due to improperly working valves, especially when long time periods pass between valve operations. The reservoir may form a fluid chamber with a variable volume, and the pump may distribute fluid from the chamber to the hydraulic means of the restriction device by reduction of the volume of the chamber and withdraws fluid from the hydraulic means to the chamber by expansion of the volume of the chamber.

The control device may reverse the operation device by shifting polarity of the energy of the second form. Where the operation device comprises an electric motor the energy of the second form suitably comprises electric energy.

In accordance with yet another embodiment of the invention, the restriction device is operable to perform a reversible function, such as enlarging and restricting the fecal passageway, and there is a reversing device implantable in the patient for reversing the function performed by the restriction device. Such a reversing function preferably involves enlarging and restricting the fecal passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gear box.

Where the reversing device comprises a switch the control device suitably controls the operation of the switch by shifting polarity of energy supplied to the switch. The switch may comprise an electric switch and the source of energy may supply electric energy for the operation of the switch.

In accordance with an advantageous embodiment of the invention, the apparatus further comprises an energy storage device implantable in the patient for storing the energy of the second form and for supplying energy in connection with the operation of the restriction device. The energy storage device preferably comprises an electric source of energy, such as an accumulator, a rechargeable battery or a combination of an accumulator and rechargeable battery.

The apparatus may further comprise a switch implantable in the patient for switching the operation of the restriction device and a source of energy implantable in the patient. This embodiment is particularly suited for applications where the energy transmission efficiency of the apparatus is insufficient, i. e., where the restriction device is to perform more advanced operations. Such a source of energy preferably is a battery. Alternatively, the source of energy is an accumulator which also may store the energy of the second form.

In accordance with a first alternative, the switch is operated by the energy of the second form supplied by the energy storage device to switch from an off mode, in which the source of energy is not in use, to an on mode, in which the source of energy supplies energy for the operation of the restriction device. In this case, the source of energy may comprise a battery, preferably having a lifetime of at least years, or an accumulator. However, other kinds of sources are also conceivable, such as a nuclear source of energy or a chemical source of energy.

In accordance with a second alternative, the apparatus further comprises a remote control for controlling the supply of energy of the source of energy, wherein the switch is operated by the energy of the second form supplied by the energy storage device to switch from an off mode, in which the remote control is prevented from controlling the source of energy and the source of energy is not in use, to a standby mode, in which the remote control is permitted to control the source of energy to supply energy for the operation of the restriction device.

In accordance with a third alternative, the energy storage device is omitted, wherein the switch is operated by the energy of the second form supplied by the energy transforming means to switch from an off mode, in which the remote control is prevented from controlling the source of energy and the source of energy is not in use, to a standby mode, in which the remote control is permitted to control the source of energy to supply energy for the operation of the restriction device.

In accordance with a fourth alternative, also the remote control is omitted, wherein the switch is operated by the energy of the second form supplied by the energy transforming means to switch from an off mode, in which the source of energy is not in use, to an on mode, in which the source of energy supplies energy for the operation of the restriction device. Where applicable, in the described embodiments the switch may switch when the energy transmission means is transmitting wireless energy, preferably while the transferred energy of the second form is stabilized by an implanted capacitor, which may temporarily (for a few seconds) store the energy of the second form.

The switch mentioned above may comprise an electronic switch or, where applicable, a mechanical switch.

The advantage of using a switch above all is increased control safety, i.e., interfering signals in the patient's surroundings cannot affect the implanted restriction device. Furthermore, the lifetime of the source of energy will be significantly prolonged, since the energy consumption of the apparatus will be reduced to a minimum. During the above-mentioned standby mode, the remote control uses energy from the implanted source of energy. By means of the energy transmission means energy may be transmitted to activate the switch to connect the source of energy only when energy is required in connection with the operation of the restriction device.

All of the above embodiments may be combined with at least one implantable sensor for sensing at least one physical parameter of the patient, wherein the control device may control the restriction device in response to signals from the sensor. For example, the sensor may comprise a pressure sensor for directly or indirectly sensing the pressure against the restriction device, human tissue or in the fecal passageway. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor. The control device may comprise an internal control unit implantable in the patient for, preferably directly, controlling the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to further restrict the fecal passageway in the colon in response to the sensor sensing that the patient is lying, or enlarge the fecal passageway in response to the sensor sensing an abnormally high pressure against the restriction device.

Alternatively, the control device may comprise an external control unit outside the patient's body for, suitably directly, controlling the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor. An external data communicator may be provided outside the patient's body and an internal data communicator may be implanted in the patient for communicating with the external data communicator. The internal data communicator may feed data related to the patient, or related to the restriction device, back to the external data communicator. Alternatively, or in combination, the external data communicator may feed data to the internal data communicator. The internal data communicator may suitably feed data related to at least one physical signal of the patient. The arrangement of external and internal data communicators gives the advantage, among other things, that a long term control of activities related to the restriction device is provided. The apparatus may further comprise an implantable programmable control unit for controlling the restriction device, preferably overtime in accordance with an activity schedule program. This will advance the apparatus and make possible an adaptation of the apparatus to the individual patients.

All of the above embodiments are preferably remote controlled. Thus, the apparatus advantageously comprises a wireless remote control transmitting at least one wireless control signal for controlling the restriction device. With such a remote control it will be possible to adapt the function of the apparatus to the patient's need in a daily basis, which is beneficial with respect to the treatment of the patient.

The wireless remote control may be capable of obtaining information on the condition of the restriction device and of controlling the restriction device in response to the information. Also, the remote control may be capable of sending information related to the restriction device from inside the patient's body to the outside thereof. In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient.

The wireless remote control may transmit a carrier signal for carrying the control signal, wherein the carrier signal is frequency, amplitude or frequency and amplitude modulated and is digital, analog or digital and analog. Also the control signal used with the carrier signal may be frequency, amplitude or frequency and amplitude modulated.

The control signal may comprise a wave signal, for example, a sound wave signal, such as an ultrasound wave signal, an electromagnetic wave signal, such as an infrared light signal, a visible light signal, an ultraviolet light signal, a laser signal, a micro wave signal, a radio wave signal, an x-ray radiation signal, or a gamma radiation signal. Where applicable, two or more of the above signals may be combined. The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

The energy transforming means may be placed in the retroperitoneal space, abdomen, scrotum or labia majora, or implanted subcutaneously. The invention also provides an implanting method, comprising the steps of providing an anal incontinence treatment apparatus as described above, cutting an opening in a patient's mucosa in an orifice of the patient's body, and implanting the energy transforming means in the patient's body through the opening. Alternatively, the cutting step may comprise cutting an opening in the patient's skin and the implanting step may comprise implanting the energy transforming means in the patient's body through the opening.

There is also provided a laparascopical implanting method, in accordance with a first alternative, comprising the steps of providing an anal incontinence treatment apparatus as described above, placing at least two laparascopic cannula within a 14 patient's body, and implanting the energy transforming means in the patient's body by using the at least two laparascopic cannula.

In accordance with another alternative there is provided a laparoscopic surgical method of implanting an anal incontinence treatment apparatus, comprising the steps of a) placing at least two laparoscopic trocars within the patient's body, b) using at least one dissecting tool inserted through the laparoscopic trocar, dissecting the region of the colon or rectum, c) introducing a restriction device of the apparatus through the trocars, d) placing the restriction device in engagement with the colon or the rectum to create a restricted stoma, and e) implanting an energy transforming means of the apparatus.

The energy transforming means of the apparatus may be implanted, for example subcutaneously, in the abdomen, thorax or cephal region, or other locations in the patient's body.

The invention also provides a method of treating a human or animal having chronic anal incontinence comprising: (a) Surgically implanting in the human or animal a restriction device engaging the human's or animal's colon or rectum, to form a restricted passageway in the colon or rectum. (b) Surgically implanting in the human or animal an operation device which can adjust the restricted passageway in response to supplied energy.

And, (c) in a non-invasive post-operative procedure, from time to time, supplying energy to the operation device so as (i) to enlarge the restricted passageway to allow feaces to readily pass.

### W: Anal Incontinence Treatment 2

According to an aspect, a new convenient anal incontinence treatment apparatus, the performance of which may be affected by the patient at any time after operation, in particular when need arise over the course of a day, so that the patient substantially always is satisfied or comfortable, is provided.

This object is obtained by an apparatus of the kind stated initially characterized by an energy transmission device for wireless transmission of energy from outside the patient's body to inside the patient's body for use in connection with the operation of the restriction device including changing the restriction of the fecal passageway, when the restriction device is implanted.

As a result, the advantage is achieved that the anal incontinence disease treatment apparatus provides simple and effective energy transmission, which ensures long reliable function of the apparatus, possibly for the rest of the patient's life.

The restriction device preferably controls the cross-sectional area of the fecal passageway in the colon or rectum, which gives the advantage that the patient is enabled to adjust the cross-sectional area of the fecal passageway, i.e. to open and close the fecal passageway, whenever the patient likes during the day.

Generally, the apparatus comprises an energy transforming device implantable in the patient for transforming the energy wirelessly transmitted by the energy transmission device from a first form into a second form, preferably different than the first form.

The energy transforming device may comprise at least one semiconductor type of component or a circuitry of such semiconductor components. The semiconductor component may comprise a transistor or microchip or similar electronic components. However, the semiconductor component may not comprise rectifying diodes.

In accordance with a main embodiment of the invention, the energy transforming device comprises at least one element having a positive region and a negative region and adapted to create an energy field between the positive and negative regions when exposed to the energy of the first form transmitted by the energy transmission device, so that the energy field provides the energy of the second form. Typically, the above-mentioned semiconductor component may include such an element.

In accordance with a preferred embodiment of the invention, the element comprises an electrical junction element capable of inducing an electric field between the positive and negative regions when exposed to the energy of the first form transmitted by the energy transmission device, whereby the energy of the second form comprises electric energy.

Consequently, the restriction device suitably is electrically operated, whereby the positive and negative regions of the electrical junction element supply electric energy for the operation of the restriction device. The apparatus suitably comprises implantable electric conductors connected to the positive and negative regions of the electrical junction element, whereby the electrical junction element is capable of supplying an electric current, such as a direct current, a pulsating direct current, a combination of a direct and pulsating direct current, an alternating current or a combination of a direct and alternating current, via the conductors. Furthermore, the electrical junction element may be capable of supplying a frequency, amplitude, or frequency and amplitude modulated analog, digital, or a combination of analog and digital signal, which is used in connection with control of the restriction device.

The element, preferably in the form of an electrical semiconductor junction element, should be designed to generate an output current exceeding 1 μA when exposed to the energy of the first form transmitted by the energy transmission device. Suitably the electrical junction element forms a flat and thin sheet and has a volume of less than 2000 cm3 to be suited for subcutaneous implantation, so that the electrical junction element can be located just behind the skin of the patient. Alternatively, it would be possible to implant the element in the thorax or cephalic region of the patient, or in an orifice of the patient's body and under the mucosa or intraluminar outside the mucosa of the orifice. Of course, all the components of the energy transforming device including the electrical junction element in contact with the patient's body should be of biocompatible material.

For in vitro appliances, a particular type of an electrical semiconductor junction element has been commonly used, namely a so-called p-n (positive/negative) junction element, typically in the form of solar cells. A solar cell transfers solar energy in the form of visible light into electric energy in the form of direct current. For example, a p-n junction element may comprise two layers of semiconductor, one p-type (positive) and the other n-type (negative), sandwiched together to form a “p-n junction”. This p-n junction induces an electric field across the element when absorbing quanta of light (photons).

To be more precise, the quanta of light transfer their energy to some of the semiconductor's electrons, which are then able to move about through the material. For each such negatively charged electron, a corresponding positive charge—a “hole”—is created. In an ordinary semiconductor, these electrons and holes recombine after a short time and their energy is wasted as heat. However, when the electrons and holes are swept across the p-n junction in opposite directions by the action of the electric field, the separation of charge induces a voltage across the p-n junction element. By connecting the p-n junction element to an external circuit, the electrons are able to flow thereby creating a current.

Surprisingly, it has been proved that although both the skin and subcutis absorb energy from an external light beam directed against the skin portion behind which a properly designed p-n junction element is located, the light energy transmitted through the skin can induce a current from the p-n junction element strong enough (minimum 1 μA) to enable the operation of the electrically operated restriction device. Thus, such a p-n junction element is now for the first time used for in vivo applications.

The apparatus may comprise an implantable pulse generator for generating electrical pulses from the energy of the second form produced by the energy field. Generally, the energy transforming device is adapted to transform the energy of the first form directly or indirectly into the energy of the second form.

In accordance with a preferred embodiment of the invention, the energy of the second form comprises electric energy and the energy transforming device comprises a capacitor, which may be adapted to produce electric pulses from the transformed electric energy. Preferably, the capacitor may be adapted to produce the pulses as the energy transforming device transforms the energy of the first form transmitted by the energy transmission device into the electric energy of the second form. The capacitor should be small to facilitate implantation thereof; i.e. its capacity may not be more than 0.1 μF.

The apparatus may comprise an implantable stabilizer for stabilizing the energy of the second form. Where the energy of the second form comprises electric current the stabilizer may comprise at least one capacitor of the type described above. In most embodiments of the invention, the apparatus comprises implantable electrical components. Where the electrical components include a capacitor of the type described above or an accumulator, at least one, preferably a single, voltage level guard may advantageously be provided, wherein the charge and discharge of the capacitor or accumulator is controlled by use of the voltage level guard. As a result, there is no need for any implanted current detector and/or charge level detector for the control of the capacitor, which makes the apparatus simple and reliable.

In a particular embodiment of the invention, the wireless energy of the first form comprises sound waves and the energy of the second form comprises electric energy, wherein the energy transforming device is adapted to directly transform the sound waves into electric energy.

The apparatus may comprise an implantable motor or pump for operating the restriction device, wherein the motor or pump is powered by the transformed energy.

In accordance with a main aspect of the invention, the energy transmission device may be adapted to transmit wireless energy for direct use in connection with the operation of the restriction device, as the wireless energy is being transmitted. The advantage of directly using energy as it is transmitted is that the apparatus can be of a very simple design and the few components involved makes the apparatus extremely reliable. For example, the energy transmission device may be adapted to directly power the motor or pump with wireless energy. The wireless energy may comprise a magnetic field or electromagnetic waves, suitably in the form of a signal, for direct power of the motor or pump. All the various functions of the motor and associated components described in the present specification may be used where applicable. As an alternative to the above-noted main aspect of the invention, the energy transforming device may be adapted to supply the energy of the second form for direct use in connection with the operation of the restriction device, as the energy of the first form is being transformed into the energy of the second form. Consequently, the energy transforming device may be adapted to directly power the motor or pump with the energy of the second form.

Generally, the energy transforming device directly operates the restriction device with the energy of the second form in a non-magnetic, non-thermal or non-mechanical manner.

Where the apparatus comprises a motor, which may be adapted to directly or intermittently operate the restriction device, the energy transforming device may power the motor with the energy of the second form. Suitably, the restriction device is operable to perform a reversible function and the motor is capable of reversing said function.

In accordance with another embodiment of the invention, the restriction device comprises a hydraulic restriction device, and the apparatus comprises an implantable pump for operating the hydraulic restriction device, wherein the energy transforming device supplies the energy of the second form for driving the pump. Preferably, the pump is not a plunger type of pump, but may comprise a peristaltic or membrane pump. The energy transforming device preferably is capable of generating as the energy of the second form a current exceeding 1 μA, when transferring the energy of the first form transmitted by the energy transmission device.

The apparatus may comprise an implantable adjustment device for adjusting the restriction device to change the restriction of the fecal passageway. In accordance with a first alternative the adjustment device is adapted to mechanically adjust the restriction device. In accordance with a second alternative the adjustment device is adapted to hydraulically adjust the restriction device by using implanted hydraulic means. Such hydraulic means may not use hydraulic fluid of the kind having a viscosity that substantially increases when exposed to heat or a magnetic field.

The apparatus of the present invention is not limited to the use of visible light for the wireless transmission of energy. Thus, in accordance with a broad aspect of the invention, the energy transmission device transmits energy by at least one wireless signal, preferably containing radiant energy.

The wireless signal may comprises a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined. Alternatively, the wave signal may comprise a sound wave signal, such as an ultrasonic signal. Generally, the wireless signal may comprise a digital, analog or a digital and analog signal.

The energy of the first form transmitted by the energy transmission device may comprise an electric or magnetic field transmitted in pulses, for example digital pulses. Furthermore, the energy transforming device may transform the energy of the first form, which may comprise polarized energy, into a direct current, pulsating direct current, a combination of a direct and pulsating direct current, an alternating current or a combination of a direct and alternating current. Alternatively, the energy of the first form may comprise kinetic energy. The energy of the second form may comprise a frequency, amplitude or frequency and amplitude modulated analog, digital or combined analog and digital signal.

The restriction device may be non-inflatable, i.e. with no hydraulic fluid involved for the adjustments of the restriction device. This eliminates problems with fluid leaking from the restriction device.

The apparatus suitably comprises implantable electric conductors connected to the energy transforming device, whereby the energy transforming device is capable of supplying an electric current, such as direct current, a pulsating direct current, a combination of a direct and pulsating direct current, an alternating current or a combination of a direct and alternating current, via the conductors. Furthermore, the energy transforming device may be capable of supplying a frequency, amplitude, or frequency and amplitude modulated analog, digital, or a combination of analog and digital signal, which is used in connection with control of the restriction device. In accordance with a main embodiment of the invention, the apparatus comprises an implantable operation device for operating the restriction device and a control device for controlling the operation device, wherein the energy transforming device powers the operation device with the energy of the second form. The operation device preferably comprises a motor, for example an electric linear motor or an electric rotary motor that is controlled by the control device to rotate c desired number of revolutions. Optionally, an implantable gearing may be connected to the motor. The electric motor may have electrically conductive parts made of plastics. Alternatively, the motor may comprise a hydraulic or pneumatic fluid motor, wherein the control device controls the fluid flow through the fluid motor. Motors currently available on the market are getting smaller and smaller. Furthermore, there is a great variety of control methods and miniaturized control equipment available. For example, the number of revolutions of a rotary motor may be analyzed by a Hall-element just a few mm in size.

In accordance with another embodiment of the invention, the restriction device comprises hydraulic means and the operation device is adapted to conduct a hydraulic fluid in the hydraulic means. The operation device comprises a fluid conduit connected to the hydraulic means of the restriction device, and a reservoir for fluid, wherein the reservoir forms part of the conduit. The reservoir may form a fluid chamber with a variable volume, and the operation device may be adapted to distribute fluid from the chamber to the hydraulic means of the restriction device by reduction of the volume of the chamber and to withdraw fluid from the hydraulic means to the chamber by expansion of the volume of the chamber. The operation device suitably comprises an implantable motor used for reducing and expanding the volume of the chamber. Also, the operation device may comprise a pump for pumping the hydraulic fluid in the hydraulic means of the restriction device. All of the hydraulic components involved are preferably devoid of any non-return valve. This is of great advantage, because with valves involved there is always a risk of malfunction due to improperly working valves, especially when long time periods passes between valve operations.

The control device may be adapted to reverse the operation device by shifting polarity of the energy of the second form. Where the operation device comprises an electric motor the energy of the second form suitably comprises electric energy. In accordance with yet another embodiment of the invention, the restriction device is operable to perform a reversible function, such as enlarging and restricting the fecal passageway, and there is a reversing device implanted in the patient for reversing the function performed by the restriction device. Such a reversing function preferably involves enlarging and restricting the fecal passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch it may be operable by the energy of the second form. In this case, the control device suitably controls the operation of the switch by shifting polarity of the energy of the second form supplied to the switch. The switch may comprise an electric switch and the source of energy may supply electric energy for the operation of the switch.

In accordance with an advantageous embodiment of the invention, the apparatus further comprises an energy storage device implanted in the patient for storing the energy of the second form and for supplying energy in connection with the operation of the restriction device. The implanted energy storage device preferably comprises an electric source of energy, such as an accumulator, a rechargeable battery or a combination of an accumulator and rechargeable battery.

The apparatus may further comprise a switch implantable in the patient for switching the operation of the restriction device and a source of energy implantable in the patient. This embodiment is particularly suited for applications where the energy transmission efficiency of the apparatus is insufficient, i.e. where the implanted restriction device is to perform more advanced operations. Such a source of energy preferably is a battery. Alternatively, the source of energy is an accumulator that also may store the energy of the second form.

In accordance with a first alternative, the switch is operated by the energy of the second form supplied by the energy storage device to switch from an off mode, in which the source of energy is not in use, to an on mode, in which the source of energy supplies energy for the operation of the restriction device. In this case, the implanted source of energy may comprise a battery, preferably having a lifetime of at least 10 years, or an accumulator. However, other kinds of sources are also conceivable, such as a nuclear source of energy or a chemical source of energy (fuel cells).

In accordance with a second alternative, the apparatus further comprises a remote control for controlling the supply of energy of the implanted source of energy, wherein the switch is operated by the energy of the second form supplied by the energy storage device to switch from an off mode, in which the remote control is prevented from controlling the source of energy and the source of energy is not in use, to a standby mode, in which the remote control is permitted to control the source of energy to supply energy for the operation of the restriction device.

In accordance with a third alternative, the energy storage device is omitted, wherein the switch is operated by the energy of the second form supplied by the energy transforming device to switch from an off mode, in which the remote control is prevented from controlling the source of energy and the source of energy is not in use, to a standby mode, in which the remote control is permitted to control the source of energy to supply energy for the operation of the restriction device.

In accordance with a fourth alternative, also the remote control is omitted, wherein the switch is operated by the energy of the second form supplied by the energy transforming device to switch from an off mode, in which the source of energy is not in use, to an on mode, in which the source of energy supplies energy for the operation of the restriction device. Where applicable, in the described embodiments the switch may switch when the energy transmission device is transmitting wireless energy, preferably while the transferred energy of the second form is stabilized by an implanted capacitor, which may temporarily (for a few seconds) store the energy of the second form.

In the above noted third and fourth alternatives, the energy transmission device may be substituted for the energy transforming device, whereby the switch is operated by the energy of the first form.

The switch mentioned above may comprise an electronic switch or, where applicable, a mechanical switch.

The advantage of using a switch above all is increased control safety; i.e. interfering signals in the patient's surroundings cannot affect the implanted restriction device. Furthermore, the lifetime of the implanted source of energy will be significantly prolonged, since the energy consumption of the apparatus will be reduced to a minimum. During the above-mentioned standby mode, the remote control uses energy from the implanted source of energy. By means of the energy transmission device energy may be transmitted to activate the switch to connect the implanted source of energy only when energy is required in connection with the operation of the restriction device.

All of the above embodiments may be combined with at least one implantable sensor for sensing at least one physical parameter of the patient, wherein the control device may control the restriction device in response to signals from the sensor. For example, the sensor may comprise a pressure sensor for directly or indirectly sensing the pressure against the restriction device, human tissue or in the fecal passageway, or the pressure against the colon or rectum. The control device may comprise an internal control unit implanted in the patient for, preferably directly, controlling the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to further restrict or close the fecal passageway in response to the sensor sensing that the patient is lying, or enlarge the fecal passageway or produce an alarm signal in response to the sensor sensing an abnormally high pressure against the restriction device.

Alternatively, the control device may comprise an external control unit outside the patient's body for, suitably directly, controlling the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor.

An external data communicator may be provided outside the patient's body and an internal data communicator may be implanted in the patient for communicating with the external communicator. The internal communicator may feed data related to the patient, or related to the restriction device, back to the external communicator. Alternatively or in combination, the external communicator may feed data to the internal communicator. The internal communicator may suitably feed data related to at least one physical signal of the patient.

The apparatus may further comprise an implantable programmable control unit for controlling the restriction device, preferably over time in accordance with an activity schedule program. This will advance the apparatus and make possible an adaptation of the apparatus to the individual patients.

Many of the above embodiments are suitably remote controlled. Thus, the apparatus advantageously comprises a wireless remote control transmitting at least one wireless control signal for controlling the restriction device. With such a remote control it will be possible to adapt the function of the apparatus to the patient's need in a daily basis, which is beneficial with respect to the treatment of the patient. The control signal may comprise a frequency, amplitude or frequency or amplitude modulated signal. Furthermore, the control signal may comprise an analog or a digital signal, or a combination of an analog and digital signal. The wireless remote control may be capable of obtaining information on the condition of the implanted restriction device and of controlling the restriction device in response to the information. Also, The remote control may be capable of sending information related to the restriction device from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient.

The wireless remote control may transmit a carrier signal for carrying the control signal, wherein the carrier signal is frequency, amplitude or frequency and amplitude modulated. The carrier signal may also comprise digital, analog or a combination of digital and analog signals. Such signals may comprise wave signals. Also the control signal used with the carrier signal may be frequency, amplitude or frequency and amplitude modulated, and be digital, analog or combined digital and analog.

The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control. The energy transmission device may function different from or similar to the energy transforming device. For example, the energy transmission and transforming devices function differently when the energy transmission device comprises a coil used for transmitting the energy of the first form and the energy transforming device comprises an electrical junction element for transforming the transmitted energy into the energy of the second form. The energy transmission and transforming devices function similar to each other when the energy transmission device comprises a coil used for transmitting the energy of the first form and the energy transforming device also comprises a coil for transforming the transmitted energy into the energy of the second form.

In accordance with an alternative embodiment of the invention, the apparatus comprises an activatable source of energy implantable in the patient, wherein the source of energy is activated by wireless energy transmitted by the energy transmission device, to supply energy which is used in connection with the operation of the restriction device. The implantable restriction device suitably is embedded in a soft or gel-like material. For example, a silicone material having hardness less than 20 Shore.

All the above described various components, such as the motor, pump and capacitor, may be combined in the different embodiments where applicable. Also the various functions described in connection with the above embodiments may be used in different applications, where applicable.

All the various ways of transferring, transforming and controlling energy presented in the present specification may be practiced by using all of the various components and solutions described.

The present invention also provides an implanting method, comprising the steps of providing an anal incontinence disease treatment apparatus as described above, cutting an opening in a patient's mucosa in an orifice of the patient's body, and implanting the energy transforming device in the patient's body through the opening. Alternatively, the cutting step may comprise cutting an opening in the patient's skin and the implanting step may comprise implanting the energy transforming device in the patient's body through the opening. There is also provided a laparascopical implanting method, in accordance with a first alternative, comprising the steps of providing an anal incontinence disease treatment apparatus as described above, placing at least two laparascopic cannula within a patient's body, and implanting the energy transforming device in the patient's body by using the at least two laparascopic cannula.

In accordance with a second alternative there is provided a laparoscopic surgical method of implanting an anal incontinence disease treatment apparatus as described above, comprising the steps of, a) placing at least two laparoscopic trocars within a patient's body, b) using at least one dissecting tool inserted through the laparoscopic trocar, dissecting the region of the colon, rectum or anus, c) introducing a restriction device of the apparatus through the trocars, and d) placing the restriction device in engagement with the colon, rectum or anus to create a restricted stoma. The method may further comprise implanting an energy transforming device of the apparatus, for example subcutaneously, in the abdomen, thorax or cephalic region, or other locations in the patient's body.

### X: Hydraulic Anal Incontinence Treatment Apparatus

According to an aspect, an anal incontinence treatment apparatus, which may be conveniently remote controlled by the patient, is provided.

These objects are obtained by an apparatus of the kind described initially characterized in that the operation device comprises a powered operation device and/or a servo means.

The expression “powered” should be understood as energized with everything without manual force, preferably electric energy. In other words, the adjustment device is operated in a non-manual manner. The expression “non-manual manner” should be understood to mean that the adjustment device is not operated by manually touching subcutaneously implanted components of the apparatus or not manipulated by touching the skin of the patient. Thus, as opposed to prior practice when treating anal incontinence, the adjustment device is not operated by manual forces, such as by manually compressing a fluid containing balloon implanted in the scrotum or in the region of labia majora. Of course, manual manipulation of a subcutaneous start button or the like for activating the powered operation device is permitted within the scope of the present invention.

Alternatively, or in combination with a powered operation device, the servo means may be used, which enables manual manipulation without need for strong manipulation forces. The servo means may comprise hydraulic means, electric control means, magnetic means, or mechanical means, which may be activated by manual manipulating means. Using a servo system will save the use of force when adjusting the adjustment device, which may be of importance in many applications. The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong. force acting on another moving element having a short stroke. The servo means may comprise a motor, preferably an electric motor, which may be reversible.

In accordance with a main embodiment of the invention, the apparatus comprises a reservoir, preferably containing a predetermined amount of hydraulic fluid, also implantable in the patient, wherein the operation device, suitably electrically powered, operates the adjustment device by using the hydraulic fluid of the reservoir. The adjustment device may comprise an expandable cavity in the restriction device, wherein the colon, rectum or anus is squeezed upon expansion of the cavity and released upon contraction of the cavity. In this embodiment the operation device is adapted to distribute hydraulic fluid from the reservoir to expand the cavity, and from the cavity to the reservoir to contract the cavity.

A fluid distribution tube may readily be connected between the reservoir and the cavity in a manner so that the. tube does not interfere with other implanted components of the apparatus.

Preferably, the reservoir defines a chamber for the predetermined amount of fluid and the operation device changes the volume of the chamber. The operation device suitably comprises first and second wall portions of the reservoir and is adapted to provide relative displacement between the first and second wall portions of the reservoir, in order to change the volume of the chamber.

The operation device may be adapted to provide said relative displacement in response to the pressure in the reservoir. Suitably, the operation device comprises a pressure controlled hydraulic operation device. For safety, an alarm may be provided for generating an alarm signal in response to the lapse of a predetermined time period during which the pressure controlling the hydraulic operation device exceeds a predetermined high value.

Suitably, the operation device is adapted to distribute fluid from the reservoir to the cavity of the restriction member in response to a predetermined first displacement of the first wall portion of the reservoir relative to the second wall portion of the reservoir and may distribute fluid from the cavity to the reservoir in response to a predetermined second displacement of the first wall portion relative to the second wall portion.

The first and second wall portions of the reservoir may be displaceable relative to each other by a magnetic, hydraulic, or electric power means, such as an electric motor. In this embodiment no pump is used, only the volume of the reservoir is varied. This is of great advantage compared to the solution described below when the operation device comprises a pump used to pump fluid between the reservoir and the adjustment device because there is no need for a non-return valve and it is still possible to have fluid going both to and from the reservoir. Thus, the significant risk of malfunction when using such a non-return valve implanted in the patient is eliminated.

The operation device may comprise hydraulic means and a fluid conduit extending between the hydraulic means and the adjustment device. The hydraulic means and conduit are devoid of any non-return valve. The reservoir may form part of the conduit and a fluid chamber with a variable volume.

The operation device may distribute fluid from the fluid chamber to the adjustment device by reduction of the volume of the chamber and withdraw fluid from the adjustment device by expansion of the volume of the chamber. The operation device preferably comprises a motor for moving a movable wall of the reservoir for changing the volume of the chamber. Any kind of motor could be used for the different operations as well as wireless remote solutions for controlling the operations. The restriction device preferably is operable to perform a reversible function and accordingly there is a reversing device implantable in the patient for reversing the function performed by the restriction device. Such a reversing function preferably involves enlarging and restricting the fecal passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch.

Where the operation device comprises a motor, the reversing device is adapted to reverse the motor. In accordance with another particular embodiment of the invention, the operation device comprises a pump for pumping fluid between the reservoir and the adjustment device. A mechanical solution is proposed in which it is possible to pump fluid from the reservoir to the adjustment device and vice versajust by pushing an activation member in one direction. The pump preferably comprises a first activation member for activating the pump to pump fluid from the reservoir to the adjustment device, and a second activation member for activating the pump to pump fluid from the adjustment device to the reservoir. At least one of the first and second activation members may be operable by manual manipulation, preferably to permit manual pushing, pulling or rotation thereof in one direction, or by a device powered magnetically, hydraulically, or electrically (e.g. by an electric motor), or be operable by a combination of these methods. Suitably, at least one of the activation members may be adapted to operate when subjected to an external pressure exceeding a predetermined magnitude.

Another alternative is a pump pumping in only one direction and an adjustable valve to change the direction of fluid to either increase or decrease the amount of fluid in the reservoir. This valve may be manipulated either manually, mechanically, magnetically, or hydraulically.

The main embodiment described above including the reservoir may alternatively be equipped with a servo means comprising a reverse servo. The term “reverse servo” is to be understood as a mechanism that transfers a strong force acting on a moving element having a short stroke into a weak force acting on another moving element having a long stroke; i.e. the reverse function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device.

The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the normal servo means and the specific reverse servo may be used in connection with all of the various components and solutions described in the present specification.

Thus, the reverse servo may be adapted to provide relative displacement between the first and second wall portions of the reservoir, suitably in response to the pressure in the reservoir, in order to change the volume of the chamber of the reservoir.

Generally, the servo means, including the reverse servo, comprises a pressure controlled servo means. The alarm mentioned above may alternatively be adapted to generate an alarm signal in response to the lapse of a predetermined time period during which the pressure controlling the servo means exceeds a predetermined high value.

The reverse servo may comprise magnetic means, electric means or manual manipulation means or a combination thereof. Preferably, however, the reverse servo comprises hydraulic means.

In accordance with a particular embodiment of the invention, the reverse servo further comprises a servo reservoir defining a chamber containing servo fluid, and the operation device comprise first and second wall portions of the servo reservoir, which are displaceable relative to each other to change the volume of the chamber of the servo reservoir. The first and second wall portions of the servo reservoir may be displaceable relative to each other by magnetic means, hydraulic means, or electric control means.

Where the reverse servo comprises hydraulic means it may further comprise a fluid supply reservoir connected to the servo reservoir in a closed system and containing a further predetermined amount of fluid. The fluid supply reservoir defines a chamber for the further predetermined amount of fluid and the operation device is adapted to change the volume of the chamber and thereby control the amount of fluid in the servo reservoir. The fluid supply reservoir comprises first and second wall portions, which are displaceable relative to each other to change the volume of the chamber of the fluid supply reservoir. Suitably, the fluid supply reservoir increases the amount of fluid in the servo reservoir in response to a predetermined first displacement of the first wall portion of the fluid supply reservoir relative to the second wall portion of the fluid supply reservoir and decreases the amount of fluid in the servo reservoir in response to a predetermined second displacement of the first wall portion of the fluid supply reservoir relative to the second wall portion of the fluid supply reservoir.

In accordance with an embodiment of the invention, the adjustment device comprises a hydraulic adjustment device, and an implantable reservoir containing a predetermined amount of hydraulic fluid and a conduit providing fluid connection between the reservoir and the hydraulic adjustment device are provided. The operation device is adapted to operate the hydraulic adjustment device by distributing hydraulic fluid through the conduit between the reservoir and the hydraulic adjustment device, wherein the conduit and hydraulic adjustment device are devoid of any non-return valve to permit free flow of hydraulic fluid in both directions in the conduit. Preferably, the reservoir forms a fluid chamber with a variable volume, and the operation device is adapted to distribute fluid from the chamber to the adjustment device by reduction of the volume of the chamber and to withdraw fluid from the adjustment device by expansion of the volume of the chamber. The operation device may comprise a motor or a pump. Alternatively, the operation device may comprise a movable wall of the reservoir for changing the volume of the chamber. For example, the operation device may be adapted to change the volume of the chamber by moving the movable wall in response to the pressure in the chamber. In the above embodiments including a reservoir for hydraulic fluid an injection port may be provided for subcutaneous implantation in the patient to be in fluid communication with the chamber of the reservoir. The injection port may be integrated in the reservoir. Such an injection port may be—provided for enabling, normally single, once-and-for-all, calibration of the amount of fluid in the hydraulic system used.

In the various embodiments hereinafter described the restriction device generally forms an at least substantially closed loop. However, the restriction device may take a variety of different shapes, such as the shape of a square, rectangle or ellipse. The substantially closed loop could for example be totally flat, i.e. thin as seen in the radial direction. The shape of restriction device may also be changed during use, by rotation or movements of the restriction device in any direction. A physical lumen, like the colon, rectum or anus, often is easier to restrict by contracting two opposite sidewalls of the lumen against each other. Thus, the restriction device may be designed to perform such a contracting effect of the opposite walls of the colon, rectum or anus. Either mechanical or hydraulic solutions may be employed to operate the restriction device. Alternatively, the restriction device may comprise an adjustable cuff, a clamp or a roller for bending or rotating the colon, rectum or anus to close its passageway. Such a cuff, clamp or roller may also be utilized for squeezing the colon, rectum or anus against human material inside the body of the patient, for example the sacral bone of the patient, or against implanted structures of the apparatus. The bending or rotating members may take any shape and be either hydraulic or non-inflatable.

Preferably the restriction device comprises an elongated restriction member and forming means for forming the restriction member into at least a substantially closed loop around the colon, rectum or anus, wherein the loop defines a restriction opening, whereby the adjustment device adjusts the restriction member in the loop to change the size of the restriction opening.

Advantageously, the forming means may form the restriction member into a loop having a predetermined size.

Alternatively, the forming means may form the restriction member into a loop having a size selected from several predetermined sizes.

The adjustment device may change the size of the restriction opening such that the outer circumferential confinement surface of the restriction member either is changed or is unchanged.

The elongated restriction member may be flexible, for example take the shape of a belt or cord, and the adjustment device may pull a first portion of the flexible restriction member from a second portion of the flexible restriction member opposite the first portion in the loop to squeeze the colon, rectum or anus between the opposite lengths of the elongated flexible restriction member to restrict the fecal passageway. The restriction member may be non-inflatable, and the adjustment device may mechanically adjust the restriction member in the loop.

The adjustment device may mechanically or hydraulically adjust the restriction device. In the embodiments described the adjustment device may either mechanically or hydraulically adjust the restriction device, where applicable. It should be noted that the operation device might mechanically or hydraulically operate the. adjustment device irrespectively of whether the adjustment device is adapted to adjust the restriction device mechanically or hydraulically.

In accordance with an embodiment of the invention, the restriction device comprises at least two elements on opposite or different sides of the colon, rectum or anus, and the adjustment device decreases the distance between the elements to squeeze the colon, rectum or anus between the elements, thereby restricting the fecal passageway. It is also possible to use only one element and squeeze the colon, rectum or anus against human bone or tissue. The elements above may as well as all the restriction members mentioned in this application be everything from rigid to soft.

In accordance with an alternative, the restriction device bends or rotates a portion of the colon, rectum or anus to restrict the fecal passageway in the same. For example, the restriction device may comprise at least two bending members, such as cylindrical or hour-glass shaped rollers, positioned on opposite or different sides of the colon, rectum or anus and displaced relative to each other along the colon, rectum or anus, and the adjustment device may move the bending members against the colon, rectum or anus to bend the latter to restrict the fecal passageway. The restriction device may also rotate a portion of the colon, rectum or anus. The bending or rotating members may take any shape and be either hydraulic or non-inflatable.

Alternatively, the two bending members one placed more distal than the other may be rotated in opposite directions relative to each other. With interconnecting means for example flexible bands between the bending members a restriction will occur between the bending members when they are rotated.

Preferably the adjustment device is operable to adjust the restriction device to steplessly change the restriction of the fecal passageway in the colon, rectum or anus. All embodiments according to the invention may be controlled by a wireless remote control.

In accordance with an advantageous embodiment of the invention, there is provided a wireless remote control for non-invasively controlling the operation device. The remote control may conveniently comprise an external hand-held remote control unit, which is manually operable by the patient to control the restriction device to squeeze and release the colon, rectum or anus. With the wireless remote control the apparatus is conveniently controlled by the patient when he so desires, which is of great advantage compared to the prior art procedures. With the remote control the apparatus is conveniently controlled to adjust the implanted restriction device to release the fecal passageway when the patient wants to relieve himself or herself.

The remote control may advantageously be capable of obtaining information related to important parameters, such as the condition of the fecal passageway or the pressure against the restriction device, and of commanding the operation device to operate the adjustment device to adjust the restriction device in response to obtained information. With the remote control the apparatus is conveniently controlled to adjust the implanted restriction device to open and close the fecal passageway. The adjustment device may control the restriction device to steplessly change the restriction of the passageway.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter. Any known or conventional signal transmitting or signal receiving means that is suitable for use with a human or mammal patient may be provided as the signal transmitter or signal receiver.

The apparatus may further comprise an implanted energizer unit for providing energy to energy consuming implanted components of the apparatus, such as electronic circuits and/or a motor for operating the adjustment device. Where a motor is provided the control unit is adapted to power the motor with energy provided by the energizer unit in response to a control signal received from the signal transmitter. The motor may be any type of motor, such as a pneumatic, hydraulic or electric motor and the energizer unit may power the motor with pressurized gas or liquid, or electric energy, depending on the type of motor. Where the motor is an electric motor, it may power pneumatic or hydraulic equipment.

The remote control advantageously comprises wireless energy transfer device for transferring energy from outside the patient's body to energy consuming implantable components of the apparatus. The energy transfer device may comprise said energizer unit is adapted to transform energy from the control signal, as it is transmitted to the signal receiver, into electric energy. Where the operation device comprises a motor the wireless energy transfer device is adapted to directly power the motor with transferred energy.

The energy transferred by the wireless energy transfer device preferably comprises a signal, suitably a wave signal. The energy transferred by the wireless energy transfer device may comprise an electric field or a magnetic field or a combination thereof. The signal may be analog or digital or a combination thereof. The energy transfer device may transfer the energy from the signal into a direct, pulsating direct or alternating current or a combination thereof.

Any of the above mentioned signals may comprise analog or digital pulses. The analog or digital signal may comprise a magnetic field or an electric field or a combination thereof. Where the signal is a wave signal it may comprise an electromagnetic wave signal, a sound wave signal or a carrier wave signal for a remote control signal or a combination thereof. Where a carrier signal is used it may be frequency, amplitude or frequency and amplitude modulated.

The apparatus may comprise an implantable source of energy for powering the operation device and/or for energizing other energy consuming components of the apparatus, wherein the energy from the source of energy is releasable from outside the patient's body. Furthermore, the apparatus may comprise an energy transmission device for wireless transmission of energy of a first form and an energy transforming device implantable in the patient for transforming the energy of the first form into energy of a second form, to be supplied to the source of energy and/or other implantable energy consuming parts of the apparatus. The energy transforming device may transform the wireless energy directly or indirectly into energy different than the wireless energy for operation of the restriction device. Typically, the energy of the second form is different than the energy of the first form. The function of the energy transmission device may be different from that of the energy transforming device.

An implantable motor or pump for operating the adjustment device may be provided, wherein the energy transmission device may be adapted to transmit wireless energy in the form of a magnetic field or electromagnetic waves or field for direct power of the motor or pump, as the wireless energy is being transmitted. Suitably, the energy transmission device transmits energy by at least one signal separate from the above mentioned control signal. An implantable stabilizer for stabilizing the energy of the first or second form may be provided. Where the energy of the second form comprises electric current, the stabilizer suitably comprises at least one capacitor.

Generally, the source of energy comprises a battery, accumulator, capacitor or a combination thereof.

In accordance with an embodiment of the invention, the apparatus comprises a control device adapted to produce wireless energy for directly powering the operation device and/or for energizing other energy consuming components of the apparatus.

Generally, the wireless energy may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal.

Any of the above mentioned signals may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal.

The control device. may be adapted to produce wireless energy in the form of a train of energy pulses and the energy transfer device may be adapted to intermittently transfer the train of energy pulses for direct use in connection with the energizing of the energy consuming components of the apparatus. Alternatively, the control device may be adapted to control the energy transforming device to produce the energy of the second form in said train of energy pulses for direct use in connection with the operation of the adjustment device. The transferred energy preferably comprises electric energy. An implantable capacitor may be provided for producing the train of energy pulses.

Where a capacitor is used in any of the above described embodiments it may have a relatively low capacity, i.e. less than 0.1 μF, in order to be small and suited for implantation.

Where the operation device comprises an implantable motor or pump for operating the adjustment device, the energy transfer device may be adapted to directly power the motor or pump with transferred energy, at the same time as the energy is transferred. Where a pump is used it should not be a plunger type of pump, because a plunger pump is noisy, but may comprise a peristaltic or membrane pump.

As mentioned above the apparatus comprises a wireless remote control for non-invasively controlling the operation device, which preferably is electrically powered. Alternatively, the operation device is powered by magnetic energy, non-magnetic energy, electromagnetic energy, non-electromagnetic energy, kinetic energy, non-kinetic energy, sonic energy, non-sonic energy, thermal energy or. non-thermal energy. However, the operation device may be unpowerable by permanent static magnetic energy. Any other kind of energy, such as electric, electromagnetic energy or a moving permanent magnetic energy, may be conceivable for operating the adjustment device. As a result, the implanted restriction device would not be accidentally adjusted if the patient comes close to any permanent magnet. Suitably, the operation device is adapted to non-invasively operate the adjustment device. Where the operation device comprises a hydraulic operation device it may use hydraulic fluid, the viscosity of which changes when the hydraulic fluid is exposed to energy, preferably electric energy, different than thermal energy. However, use of hydraulic fluid of the kind having a viscosity that substantially increases when exposed to heat or a magnetic field, i.e. the hydraulic fluid would not become more viscous when exposed to heat or influenced by magnetic forces, should be avoided, because external heat sources or heat from the body when the patient has fever and external magnetic sources might affect the implanted components of the apparatus.

The adjustment device is may be operable to adjust the restriction device to steplessly change the restriction of the fecal passageway. Furthermore, the adjustment device may be adapted to mechanically adjust the restriction device. Alternatively, it may be adapted to hydraulically adjust the restriction device by using hydraulic means, which is devoid of hydraulic fluid of the kind having a viscosity that substantially increases when exposed to heat or a magnetic field.

In accordance with an embodiment of the invention, the apparatus comprises a control device for controlling the restriction device. The control device may comprise an internal programmable control unit implantable in the patient and, possibly an external control unit outside the patient's body for programming the programmable internal control unit. Alternatively, the external control unit may be programmable and wirelessly. control the restriction device. The control device may be adapted to produce wireless energy for directly powering the operation device and/or for energizing other energy consuming components of the apparatus.

At least one sensor. for sensing at least one physical parameter of the patient may conveniently be implanted in the patient. The sensor may preferably sense as the physical parameter the horizontal position of the patient or may comprise a pressure sensor for sensing the pressure against the restriction device or the colon, rectum or anus or other important parameters. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor. Either the internal control unit or the external control unit of the control device may suitably control the restriction device to enlarge or close the fecal passageway. For safety the restriction device may enlarge or open the fecal passageway in response to the sensor sensing for example an abnormally high pressure value. The internal control unit may directly control the restriction device in response to signals from the sensor.

Wherever magnetic means is utilized according to the invention it may comprise a permanent magnet and a magnetic material reed switch, or other suitable known or conventional magnetic means.

Where a source of energy is used the control device suitably is operable from outside the patient's body for controlling the source of energy to release energy for use in connection with the operation of the adjustment device, when the adjustment device is implanted. The source of energy may be provided external to the patient's body, and the control device may be adapted to control the external source of energy to release wireless energy for use in connection with the operation of the adjustment device.

The control device may control the source of energy to release magnetic energy, non-magnetic energy, electromagnetic energy, non-electromagnetic energy, kinetic energy, non-kinetic energy, sonic energy, non-sonic energy, thermal energy or non-thermal energy, preferably in a non-invasive manner and for a determined time period and/or in a determined number of energy pulses.

Where the implantable components of the apparatus comprise electrical components they may include at least one or a single voltage level guard. In this case, the electrical components suitably are devoid of any current detector and/or charge level detector. Furthermore, the electrical components may comprise a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

In accordance with an advantageous embodiment of the invention, the apparatus comprises an implantable switch for directly or indirectly switching the operation of the restriction device. The switch may be operated by the energy supplied by the energy transmission device mentioned above to switch from an off mode, in which the implantable source of energy mentioned above is not in use, to an on mode, in which the source of energy supplies energy for the operation of the restriction device.

In accordance with an alternative embodiment, the above mentioned a remote control may be employed for controlling the implantable source of energy, wherein the switch is operated by the energy supplied by the energy transmission device to switch from an off mode, in which the remote control is prevented from controlling the source of energy and the source of energy is not in use, to a standby mode, in which the remote control is permitted to control the source of energy to supply energy for the operation of the restriction device.

In accordance with another alternative embodiment, the switch is operated by the energy supplied by the implantable energy transforming device mentioned above to switch from an off mode, in which the source of energy is not in use, to an on mode, in which the source of energy supplies energy for the operation of the restriction device.

In accordance with yet another alternative embodiment, the switch is operated by the energy supplied by the energy transforming device to switch from an off mode, in which the remote control is prevented from controlling the source of energy and the source of energy is not in use, to a standby mode, in which the remote control is permitted to control the source of energy to supply energy for the operation of the restriction device.

Suitably, the restriction device is embedded in a soft or gel-like material, such as a silicone material having hardness less than 20 Shore. The energy transforming device may be designed to be implanted subcutaneously or in the abdomen, thorax or cephalic region of the patient.

The adjustment device may be adapted to adjust the restriction device such that the restriction device provides a predetermined contraction of the fecal passageway that is satisfactory for the patient.

All the above described various components, such as the motor, pump and capacitor, may be combined in the different embodiments where applicable. Also the various functions described in connection with the above embodiments may be used in different applications, where applicable. Specifically, the various remote control functions described and all the various methods for supplying energy may be used in any conceivable combination that is apparent to those skilled in the art.

The invention also provides a method for treating a patient suffering from anal incontinence, comprising the steps of placing at least two laparascopical trocars in the body of a patient suffering from anal incontinence, inserting a dissecting tool through the trocars, and dissecting an area of the colon, rectum or anus in the abdominal or pelvic or retroperitoneal surroundings, placing at least one adjustable restriction device in the dissected area engaging the rectum or colon, adjusting the restriction device to normally restrict the fecal passageway in the rectum or colon, and adjusting the restriction device to open the fecal passageway when the patient wants to relieve himself or herself. A hydraulic adjustable restriction device may be used when practicing this method, in a non-manual manner, i.e. without touching subcutaneously implanted components of the apparatus or by using power means that is manually activated. The method may further comprise implanting a source of energy in the patient and providing a control device for controlling the source of energy from outside the patient's body to supply energy for the adjustment of the restriction device. The present invention also provides a method for treating anal incontinence, comprising surgically implanting in the body of a patient suffering from anal incontinence an adjustable restriction device engaging the colon, rectum or anus to restrict the fecal passageway, and when desired to allow the patient to relieve himself or herself, adjusting the restriction device to temporarily release the colon, rectum or anus to open the fecal passageway. The method may further comprise implanting an elongated restriction member of the restriction device around the colon, rectum or anus.

The anal incontinence treatment apparatus may also be laparoscopicly implanted. Thus, there is provided a method comprising the steps of placing at least two laparascopical trocars in the patient's body, inserting a dissecting tool through the trocars and dissecting an area of the pelvic or abdominal or retroperitoneal surroundings and placing an operable restriction device in the dissected area, so that the restriction device engages the colon, rectum or anus to restrict the fecal passageway. Additionally, the method may comprise implanting an energy transfer device of the apparatus.

### Y: Anal Incontinence Treatment with Controlled Wireless Energy Supply

According to an aspect, an anal incontinence treatment apparatus, comprising a restriction device implantable in a patient suffering from anal incontinence for engaging the colon or rectum to form a restricted fecal passageway in the colon or rectum, wherein the restriction device is operable to change the restriction of the fecal passageway, is provided. The term “patient” includes an animal or a human being.

Anal incontinence is a wide-spread disease. Several kinds of sphincter plastic surgery are used today to remedy anal incontinence. There is a prior manually operated sphincter system in an initial clinical trial phase where a hydraulic sphincter system connected to an elastic reservoir (balloon) placed in the scrotum is developed. A disadvantage of this system is that thick, hard fibrosis is created around the reservoir by pump movements making the system useless sooner or later.

U.S. Pat. No. 5,593,443 discloses a hydraulic anal sphincter under both reflex and voluntary control. A pressure controlled inflatable artificial sphincter is disclosed in U.S. Pat. No. 4,222,377.

The object of the present invention is to provide a new convenient anal incontinence treatment apparatus, the performance of which may be affected by the patient at any time after operation, in particular when need arise over the course of a day, so that the patient substantially always is satisfied or comfortable. This object is achieved by an anal incontinence treatment apparatus of the kind stated initially, which is characterized in that a source of energy is provided, and a control device operable from outside the patient's body is provided for controlling the source of energy to release energy for use in connection with the operation of the restriction device, when the restriction device is implanted. As a result, the advantage is achieved that the restriction device can be non-invasively operated, when the restriction device has to be adjusted. Furthermore, the apparatus provides a simple and effective control of the energy supplied to implanted components of the apparatus which ensures an extended and reliable functionality of the apparatus, possibly for the rest of the patient's life and at least many years.

The control device may also control the restriction device. The control device may comprise an internal control unit, preferably including a microprocessor, implantable in the patient for controlling the restriction device. The control device may further comprise an external control unit outside the patient's body, wherein the internal control unit is programmable by the external control unit, for example for controlling the restriction device over time. Alternatively, the internal control unit may control the restriction device over time in accordance with an activity schedule program, which may be adapted to the patient's needs.

A great advantage is that the patient is enabled to adjust the restriction of the fecal passageway by using the control device whenever he likes during the day.

Conveniently, the external control unit may load the internal control unit with data in accordance with a loading mode only authorized for a doctor. For specialized controls of the restriction device, the external control unit may control the internal control unit in accordance with a doctor mode only authorized for the doctor. For simple controls of the restriction device, the external control unit may control the internal control unit in accordance with a patient mode permitted for the patient. Thus, by using the external control unit in accordance with different modes it is possible to have certain functions of the restriction device controlled by the patient and other more advanced functions controlled by the doctor, which enables a flexible post-operation treatment of the patient.

The control device may be adapted to control the source of energy to release energy, for instance to intermittently release energy in the form of a train of energy pulses, for direct use in connection with the operation of the restriction device. In accordance with a suitable embodiment the control device controls the source of energy to release electric energy, and the apparatus further comprises an implantable capacitor for producing the train of energy pulses from the released energy. In this case the term “direct” is used to mean, on one hand, that the released energy is used while it is being released by the control device, on the other hand, that the released energy may be somewhat delayed, in the order of seconds, by for instance an energy stabilizer before being used in connection with the operation of the restriction device. The restriction device may be operable in non-manual, a non-magnetic or non-mechanical manner by use of the released energy.

In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

Generally, the apparatus further comprises an operation device implantable in the patient for operating the restriction device, wherein the control device controls the operation device to operate the restriction device. The control device may directly power the operation device with energy released from the source of energy and/or power other implantable energy consuming components of the apparatus. In this case the term “directly” is used to mean, on one hand, that the operation device is powered with released energy while the latter is being released by the control device, on the other hand, that the released energy may be somewhat delayed, in the order of seconds, by for instance an energy stabilizer before powering the operation device. The advantage of directly using energy as it is released is that the apparatus can be of a very simple design and the few components involved makes the apparatus reliable. The restriction device may be non-inflatable, i.e. with no hydraulic fluid involved for the adjustments of the restriction device. This eliminates problems with fluid leaking from the restriction device.

The operation device may comprise hydraulic means and at least one valve for controlling a fluid flow in the hydraulic means. The control device may suitably comprise a wireless remote control for controlling the valve. The restriction device may comprise hydraulic means and the operation device may comprise a reservoir forming a fluid chamber with a variable volume connected to the hydraulic means. The operation device may distribute fluid from the chamber to the hydraulic means by reduction of the volume of the chamber and withdraw fluid from the hydraulic means to the chamber by expansion of the volume of the chamber. In accordance with a first main aspect of the invention, the source of energy is external to the patient's body and the control device controls the source of energy to release wireless energy. The external source of energy may be of any conceivable kind, such as a nuclear source of energy or a chemical source of energy.

An energy storage device, preferably an electric accumulator, may be implantable in the patient for storing the wireless energy released from the external source of energy. The electric accumulator may comprise at least one capacitor or at least one rechargeable battery, or a combination of at least one capacitor and at least one rechargeable battery. Alternatively, a battery may be implantable in the patient for supplying electric energy to implanted electric energy consuming components of the apparatus, in addition to the supply of wireless energy. Where the control device comprises an implantable control unit the electronic circuit thereof and the restriction device may be directly powered with transformed wireless energy, or energy from either the implantable energy storage device or battery.

In accordance with a second main aspect of the invention, the wireless energy is directly used for operation of the restriction device, i.e. the restriction device is operated as the wireless energy is released from the external source of energy by the control device. In this case the term “directly” is used to mean, on one hand, that the restriction device is promptly operated by using the released energy without first storing the latter, on the other hand, that the released energy may be somewhat delayed, in the order of seconds, by for instance an energy stabilizer before being used for the operation of the restriction device. As a result, a very simple control of the restriction device is achieved and there are only a few implanted components of the apparatus. For example, there is no implanted source of energy, such as a battery, nor any implanted complicated signal control system. This gives the advantage that the apparatus will be extremely reliable. Generally, the control device controls and directly or indirectly powers the operation device with wireless energy released from the source of energy and/or powers other implanted energy consuming components of the apparatus. In a first particular embodiment in accordance with the first and second main aspects of the invention, the operation device comprises a motor, preferably an electric motor which may have electrically conductive parts made of plastics. The motor may include a rotary motor, wherein the control device is adapted to control the rotary motor to rotate a desired number of revolutions. Alternatively, the motor may include a linear motor, or a hydraulic or pneumatic fluid motor, wherein the control device is adapted to control the fluid flow through the fluid motor. Motors currently available on the market are getting smaller and smaller. Furthermore, there is a great variety of control methods and miniaturized control equipment available. For example, a number of revolutions of a rotary motor may be analyzed by a Hall-element just a few mm in size.

In a second particular embodiment in accordance with the first and second main aspects of the invention, the control device is adapted to shift polarity of the released energy to reverse the operation device. The operation device may suitably comprise an electric motor and the released energy may comprise electric energy. In a third particular embodiment in accordance with the first and second main aspects of the invention, the restriction device is operable to perform a reversible function and there is a reversing device implantable in the patient for reversing the function performed by the restriction device. Such a reversing function preferably involves enlarging and restricting the fecal passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch.

In accordance with the third particular embodiment, the operation device preferably comprises a motor, wherein the reversing device reverses the motor.

In a fourth particular embodiment in accordance with the first and second main aspects of the invention, the restriction device comprises hydraulic means, for example including an expansible/contractible cavity for fluid. Preferably, the operation device is adapted to conduct hydraulic fluid in the hydraulic means, and comprises a motor, a valveless fluid conduit connected to the hydraulic means of the restriction device, and a reservoir for fluid, wherein the reservoir forms part of the conduit. The operation device suitably comprises a pump operated by the motor. All of the hydraulic components involved are preferably devoid of any non-return valve. This is of great advantage, because with valves involved there is always a risk of malfunction due to improperly working valves, especially when long time periods passes between valve operations. The reservoir may form a fluid chamber with a variable volume, and the pump may distribute fluid from the chamber to the hydraulic means of the restriction device by reduction of the volume of the chamber and withdraw fluid from the hydraulic means to the chamber by expansion of the volume of the chamber.

In accordance with a third main aspect of the invention, the source of energy is implantable in the patient. Thus, when the source of energy is implanted in a patient the control device controls it from outside the patient's body to release energy. This solution is advantageous for embodiments of the apparatus that have a relatively high consumption of energy which cannot be satisfied by direct supply of wireless energy.

The implantable source of energy may comprise an accumulator, preferably an electric source of energy, such as a battery having a lifetime of at least 10 years.

In accordance with a fourth main aspect of the invention, the apparatus comprises a switch implanted in the patient for directly or indirectly switching the operation of the restriction device and an internal source of energy, such as a battery, implanted in the patient for supplying energy for the operation of the restriction device, wherein the switch directly or indirectly affects the supply of energy from the internal source of energy. This solution is advantageous for embodiments of the apparatus that have a relatively high energy consumption which cannot be met by direct supply of wireless energy.

In a first particular embodiment in accordance with the fourth main aspect of the invention, the switch switches between an off mode, in which the internal source of energy is not in use, and an on mode, in which the internal source of energy supplies energy for the operation of the restriction device. In this case, the switch is conveniently operated by the wireless energy released from the external source of energy to switch between the on and off modes. The control device, preferably comprising a wireless remote control, may control the external source of energy to release the wireless energy. The advantage of this embodiment is that the lifetime of the implanted source of energy, such as a battery, can be significantly prolonged, since the implanted source of energy does not supply energy when the switch is in its off mode.

In a second particular embodiment in accordance with the fourth main aspect of the invention, the control device comprises a wireless remote control for controlling the internal source of energy. In this case, the switch is operable by the wireless energy from the external source of energy to switch between an off mode, in which the internal source of energy and remote control are not in use, and a standby mode, in which the remote control is permitted to control the internal source of energy to supply energy for the operation of the restriction device. In a third particular embodiment in accordance with the fourth main aspect of the invention, the apparatus further comprises an energy transforming device implantable in the patient for transforming the wireless energy into storable energy, wherein the internal source of energy is capable of storing the storable energy. The internal source of energy preferably comprises an electric accumulator, at least one capacitor or at least one rechargeable battery, or a combination of at least one capacitor and at least one rechargeable battery. In this case, the switch switches from an off mode, in which the internal source of energy is not in use, to an on mode, in which the internal source of energy supplies energy for the operation of the restriction device.

The control device, preferably comprising a wireless remote control, may control the switch to switch between the on and off modes.

Alternatively, in this third particular embodiment an energy storage device may be implanted in the patient for storing the storable energy instead of the internal source of energy, wherein the switch is operable by energy from the implanted energy storage device to switch between an off mode, in which the internal source of energy is not in use, and an on mode, in which the internal source of energy supplies energy for the operation of the restriction device. In this case, the control device (the wireless remote control) controls the energy storage device to operate the switch.

The internal source of energy preferably comprises an electric source of energy, such as an accumulator or a battery having a lifetime of at least 10 years. However, other kinds of sources are also conceivable, such as a nuclear source of energy or a chemical source of energy.

The above first, second, third and fourth particular embodiments described in connection with the first and second main aspects are also applicable in accordance with the third main aspect of the invention, i.e. where the source of energy is implantable, and in accordance with the fourth main aspect of the invention, i.e. where the apparatus comprises an implantable switch.

All of the above embodiments may be combined with at least one implantable sensor for sensing at least one physical parameter of the patient, wherein the control device may control the restriction device in response to signals from the sensor. For example, the sensor may comprise a pressure sensor for directly or indirectly sensing the pressure in the colon or rectum. The expression “indirectly sensing the pressure in the colon or rectum” should be understood to encompass the cases where the sensor senses the pressure against the restriction device or human tissue of the patient. Where the control device comprises an internal control unit to be implanted in the patient, the internal control unit may suitably directly control the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to firmly close the fecal passageway in response to the sensor sensing that the patient is lying, or enlarge the fecal passageway in response to the sensor sensing an abnormally high pressure against the restriction device.

Where the control device comprises an external control unit outside the patient's body, the external control unit may, suitably directly, control the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor.

An external data communicator may be provided outside the patient's body and an internal data communicator to be implanted in the patient may be provided for communicating with the external data communicator. The internal data communicator may feed data related to the patient, or related to the restriction device, back to the external data communicator. Alternatively or in combination, the external data communicator may feed data to the internal data communicator. The internal data communicator may suitably feed data related to at least one physical signal of the patient.

Generally, the apparatus may comprise a switch implantable in the patient for directly or indirectly switching the energy released from the source of energy. For example, the restriction device may be operable to open and close the fecal passageway or may steplessly control the restriction of the fecal passageway. A pressure sensor may be provided for directly or indirectly sensing the pressure in the colon or rectum. The control device may control the restriction device in response to signals from the pressure sensor.

The apparatus may comprise an implantable energy transforming device, wherein the control device releases electric energy and the energy transforming device transforms the electric energy into kinetic energy for, preferably direct, operation of the restriction device. Suitably, an implantable stabilizer, such as a capacitor or a rechargeable accumulator, or the like, may be provided for stabilizing the electric energy released by the control device. In addition, the control device may control the source of energy to release energy for a determined time period or in a determined number of energy pulses. Finally, the restriction device may be non-inflatable.

All of the above embodiments are preferably remote controlled. Thus, the control device advantageously comprises a wireless remote control transmitting at least one wireless control signal for controlling the restriction device. With such a remote control it will be possible to adapt the function of the apparatus to the patient's need in a daily basis, which is beneficial with respect to the treatment of the patient.

The wireless remote control may be capable of obtaining information on the condition of the restriction device and of controlling the restriction device in response to the information. Also, The remote control may be capable of sending information related to the restriction device from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient.

The remote control may transmit a carrier signal for carrying the control signal, wherein the carrier signal is frequency, amplitude or frequency and amplitude modulated and is digital, analog or digital and analog. Also the control signal used with the carrier signal may be frequency, amplitude or frequency and amplitude modulated.

The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

In all of the above solutions, the control device advantageously releases energy from the source of energy in a non-invasive, magnetic, non-magnetic, mechanical or non-mechanical manner.

The control device may release magnetic, electromagnetic, kinetic, sonic or thermal energy, or non-magnetic, non-sonic, non-thermal, non-electromagnetic or non-kinetic energy.

The control device may be activated in a manual or non-manual manner to control the source of energy to release energy. The operation device may be powered by magnetic energy, nonmagnetic energy, electromagnetic energy, non-electromagnetic energy, kinetic energy, non-kinetic energy, thermal energy or non-thermal energy. However, preferably the operation device comprises an electrical operation device. Typically the apparatus comprises an adjustment device for adjusting the restriction device between erect and flaccid penile conditions. The adjustment device may be adapted to mechanically adjust the restriction device. Alternatively, the adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces. The above-presented embodiments may be modified in accordance with the following suggestions. The released energy may comprise electric energy and an implantable capacitor having a capacity less than 0.1 μF may be provided for producing the above-mentioned train of energy pulses. An implantable motor or pump may be provided for operating the restriction device, wherein the control device is adapted to control the source of energy to directly power the motor or pump with the released energy. Specifically, the control device may be adapted to release wireless energy in the form of a magnetic field or electromagnetic waves (excluding radio waves) for direct power of the motor or pump, as the wireless energy is being released. Where a pump is used it preferably is not a plunger type of pump.

Generally, the wireless energy comprises a signal.

The apparatus may further comprise implantable energy transforming device for transforming wireless energy directly or indirectly into energy different than the wireless energy, for operation of the restriction device. For example, the motor or pump may be powered by the transformed energy.

The energy transforming device may transform the wireless energy in the form of sound waves, preferably directly, into electric energy for operation of the restriction device. The energy transforming device may comprise a capacitor adapted to produce electric pulses from the transformed electric energy.

The motor mentioned in the present specification may also be directly powered with wirelessly transmitted electromagnetic or magnetic energy in the form of signals, as the energy is transmitted. Furthermore, all the various functions of the motor and associated components described in the present specification may be used where applicable.

Generally, the restriction device advantageously is embedded in a soft or gel-like material, such as a silicone material having hardness less than 20 Shore.

Of course, the restriction device preferably is adjustable in a non-manual manner.

All the above described various components, such as the motor, pump and capacitor, may be combined in the different embodiments where applicable. Also the various functions described in connection with the above embodiments may be used in different applications, where applicable. All the various ways of transferring energy and controlling the energy presented in the present specification may be practiced by using all of the various components and solutions described.

The present invention also provides methods for treating anal incontinent patients.

Accordingly, in accordance with a first alternative method, there is provided a method of treating a patient suffering from anal incontinence, comprising the steps of implanting an operable restriction device in the patient, so that the restriction device engages the colon or rectum to form a restricted fecal passageway in the colon or rectum, providing a source of energy for energizing the restriction device, and controlling the source of energy to release energy for use in connection with the operation of the restriction device. The method may further comprise using energy released from the source of energy to operate the restriction device to open and close, respectively, the fecal passageway.

In accordance with a second alternative method, there is provided a method of treating a patient suffering from anal incontinence, comprising the steps of placing at least two laparascopical trocars in the patient's body, inserting a dissecting tool through the trocars and dissecting an area of the colon or rectum, placing an operable restriction device in the dissected area, so that the restriction device engages the colon or rectum to form a restricted fecal passageway in the colon or rectum, implanting a source of energy in the patient, and controlling the implanted source of energy from outside the patient's body to release energy for use in connection with the operation of the restriction device.

In accordance with a third alternative method, there is provided a method of treating a patient suffering from anal incontinence, comprising: (a) Surgically implanting in the patient an operable restriction device engaging the patient's colon or rectum to form a restricted fecal passageway in the colon or rectum, (b) Providing a source of energy external to the patient's body. (c) Controlling the external source of energy from outside the patient's body to release wireless energy. And (d) using the released wireless energy in connection with the operation of the restriction device.

The method may further comprise (e) implanting in the human or animal an operation device which can adjust the restricted fecal passageway in response to supplied energy, and (f) using the released wireless energy to activate the implanted operation device so as (i) to enlarge the restricted fecal passageway to allow feaces to readily pass therethrough but normally restrict the fecal passageway.

In accordance with a fourth alternative method, there is provided a method of treating a patient suffering from anal incontinence, comprising the steps of placing at least two laparascopical trocars in the patient's body, inserting a dissecting tool through the trocars and dissecting an area of the colon or rectum, placing an operable restriction device in the dissected area, so that the restriction device engages the colon or rectum to form a restricted fecal passageway in the colon or rectum, providing an external source of energy outside the patient's body, controlling the external source of energy from outside the patient's body to release wireless energy, and using the released wireless energy in connection with the operation of the restriction device.

In accordance with a fifth alternative method, there is provided a method of treating a patient suffering from anal incontinence, comprising the steps of placing at least two laparascopical trocars in the patient's body, inserting a dissecting tool through the trocars and dissecting an area of the colon or rectum, implanting an operable restriction device in the dissected area, so that the restriction device engages the colon or rectum to form a restricted fecal passageway in the colon or rectum, implanting an energy transforming device, providing an external source of energy, controlling the external source of energy to release wireless energy, and transforming the wireless energy by the energy transforming device into energy different than the wireless energy for use in connection with the operation of the restriction device. This method may further comprise implanting a stabilizer in the patient for stabilizing the energy transformed by the energy transforming device.

### Z: Impotence Treatment

The present aspect relates to a male sexual impotence treatment apparatus, comprising an operable restriction device implantable in an impotent patient for engaging a tissue portion of the normal penile tissue or the prolongation thereof to form a restrictable penile exit blood flow passageway.

The expression “normal penile tissue or the prolongation thereof” should be understood to mean the penile tissue extended inside the human body and following the pathway of the blood flow leaving the penis i.e. one or more exit veins from the penis, the corpus cavernosum, crura or the prolongation thereof. Male sexual impotence is a widespread problem. Many different solutions to this problem have been tried. A solution currently practiced is to replace the corpus cavernosa in the penis with a hydraulic inflatable/contractable silicon prosthesis, thus implanted in the penis. In fluid connection with this prosthesis is a reservoir implanted in the scrotum. By manual pumping action the prosthesis is filled with fluid from the reservoir to effect erect penile condition or is emptied of fluid, which returns to the reservoir, to effect flaccid penile condition. However, there are several more or less severe disadvantages of this solution. Above all, the penis is more or less damaged by the operation and it is practically impossible to reverse the operation. Another disadvantage is that rather strong forces act against this implanted prosthesis resulting in a significant risk of the prosthesis being broken.

Another solution to achieve erection is to restrict the blood flow leaving the penis. For example, U.S. Pat. Nos. 4,829,990, 4,958,630 and 5,048,511 disclose two hydraulically operated inflatable cuffs wrapped around respective crura or veins. A disadvantage of such a solution is that it involves complicated surgery. Another example on this solution is U.S. Pat. No. 4,828,544, which discloses an artificial fistula system surgically implanted and providing a primary fistula between the femoral artery and the femoral vein and a secondary fistula for leading blood from the primary fistula to the penis. The inflatable balloon engages the primary fistula between the secondary fistula and the vein. The balloon is in fluid connection with a manually compressible reservoir implanted in the scrotum. Again, implantation of this artificial fistula system requires delicate surgery. Yet another solution is to inject a substance in the penile vein system to achieve erection. However, injections are painful and complicated for the patient.

The object of the present invention is to provide a simple male sexual impotence treatment apparatus, which is conveniently controlled by the patient.

This object is obtained by an apparatus of the kind stated initially characterized by an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.

As a result, the advantage is achieved that the impotence treatment apparatus provides simple and J effective energy transmission which ensures an extended and reliable functionality of the apparatus, possibly for the rest of the patient's natural life, and at least many years.

The restriction device preferably controls the cross-sectional area of the passageway through which blood flow leaves the penis, which gives the advantage that the patient is enabled to adjust the restriction device to achieve the desired erection without feeling pain. This advantage should not be underestimated, because fine adjustments to restrict the cross-sectional area of the passageway, will allow maximal erection with minimum of restriction.

Generally, the apparatus comprises an energy transforming device implantable in the patient for transforming the energy wirelessly transmitted by the energy transmission device from a first form into a second form, preferably different than the first form.

The energy transforming device may comprise at least one semiconductor type of component or a circuitry of such semiconductor components. The semiconductor component may comprise a transistor or microchip or similar electronic components. However, the semiconductor component may not comprise rectifying diodes.

In accordance with a main embodiment of the invention, the energy transforming device comprises at least one element having a positive region and a negative region and adapted to create an energy field between the positive and negative regions when exposed to the energy of the first form transmitted by the energy transmission device, so that the energy field provides the energy of the second form. Typically, the above-mentioned semiconductor component may include such an element.

In accordance with a preferred embodiment of the invention, the element comprises an electrical junction element capable of inducing an electric field between the positive and negative regions when exposed to the energy of the first form transmitted by the energy transmission device, whereby the energy of the second form comprises electric energy.

Consequently, the restriction device suitably is electrically operated, whereby the positive and negative regions of the electrical junction element supply electric energy for the operation of the restriction device. The apparatus suitably comprises implantable electric conductors connected to the positive and negative regions of the electrical junction element, whereby the electrical junction element is capable of supplying an electric current, such as a direct current, a pulsating direct current, a combination of a direct and pulsating direct current, an alternating current or a combination of a direct and alternating current, via the conductors. Furthermore, the electrical junction element may be capable of supplying a frequency, amplitude, or frequency and amplitude modulated analog, digital, or a combination of analog and digital signal, which is used in connection with control of the restriction device.

The element, preferably in the form of an electrical semiconductor junction element, should be designed to generate an output current exceeding 1 μA when exposed to the energy of the first form transmitted by the energy transmission device. Suitably the electrical junction element forms a flat and thin sheet and has a volume of less than 2000 cm3 to be suited for subcutaneous implantation, so that the electrical junction element can be located just behind the skin of the patient. Alternatively, it would be possible to implant the element in the thorax or cephalic region of the patient, or in an orifice of the patient's body and under the mucosa or intraluminar outside the mucosa of the orifice. Of course, all the components of the energy transforming device including the electrical junction element in contact with the patient's body should be of biocompatible material.

For in vitro appliances, a particular type of an electrical semiconductor junction element has been commonly used, namely a so-called p-n (positive/negative) junction element, typically in the form of solar cells. A solar cell transfers solar energy in the form of visible light into electric energy in the form of direct current. For example, a p-n junction element may comprise two layers of semiconductor, one p-type (positive) and the other n-type (negative), sandwiched together to form a “p-n junction”. This p-n junction induces an electric field across the element when absorbing quanta of light (photons).

To be more precise, the quanta of light transfer their energy to some of the semiconductor's electrons, which are then able to move about through the material. For each such negatively charged electron, a corresponding positive charge—a “hole”—is created. In an ordinary semiconductor, these electrons and holes recombine after a short time and their energy is wasted as heat. However, when the electrons and holes are swept across the p-n junction in opposite directions by the action of the electric field, the separation of charge induces a voltage across the p-n junction element. By connecting the p-n junction element to an external circuit, the electrons are able to flow thereby creating a current.

Surprisingly, it has been proved that although both the skin and subcutis absorb energy from an external light beam directed against the skin portion behind which a properly designed p-n junction element is located, the light energy transmitted through the skin can induce a current from the p-n junction element strong enough (minimum 1 μA) to enable the operation of the electrically operated restriction device. Thus, such a p-n junction element is now for the first time used for in vivo applications.

The apparatus may comprise an implantable pulse generator for generating electrical pulses from the energy of the second form produced by the energy field.

Generally, the energy transforming device is adapted to transform the energy of the first form directly or indirectly into the energy of the second form.

In accordance with a preferred embodiment of the invention, the energy of the second form comprises electric energy and the energy transforming device comprises a capacitor, which may be adapted to produce electric pulses from the transformed electric energy. Preferably, the capacitor may be adapted to produce the pulses as the energy transforming device transforms the energy of the first form transmitted by the energy transmission device into the electric energy of the second form. The capacitor should be small to facilitate implantation thereof; i.e. its capacity may not be more than 0.1 μF.

The apparatus may comprise an implantable stabilizer for stabilizing the energy of the second form. Where the energy of the second form comprises electric current the stabilizer may comprise at least one capacitor of the type described above.

In most embodiments of the invention, the apparatus comprises implantable electrical components. Where the electrical components include a capacitor of the type described above or an accumulator, at least one, preferably a single, voltage level guard may advantageously be provided, wherein the charge and discharge of the capacitor or accumulator is controlled by use of the voltage level guard. As a result, there is no need for any implanted current detector and/or charge level detector for the control of the capacitor, which makes the apparatus simple and reliable. In a particular embodiment of the invention, the wireless energy of the first form comprises sound waves and the energy of the second form comprises electric energy, wherein the energy transforming device is adapted to directly transform the sound waves into electric energy. The apparatus may comprise an implantable motor or pump for operating the restriction device, wherein the motor or pump is powered by the transformed energy.

In accordance with a main aspect of the invention, the energy transmission device may be adapted to transmit wireless energy for direct use in connection with the operation of the restriction device, as the wireless energy is being transmitted. The advantage of directly using energy as it is transmitted is that the apparatus can be of a very simple design and the few components involved makes the apparatus extremely reliable. For example, the energy transmission device may be adapted to directly power the motor or pump with wireless energy. The wireless energy may comprise a magnetic field or electromagnetic waves, suitably in the form of a signal, for direct power of the motor or pump. All the various functions of the motor and associated components described in the present specification may be used where applicable.

As an alternative to the above-noted main aspect of the invention, the energy transforming device may be adapted to supply the energy of the second form for direct use in connection with the operation of the restriction device, as the energy of the first form is being transformed into the energy of the second form. Consequently, the energy transforming device may be adapted to directly power the motor or pump with the energy of the second form.

Generally, the energy transforming device directly operates the restriction device with the energy of the second form in a non-magnetic, non-thermal or non-mechanical manner.

Where the apparatus comprises a motor, which may be adapted to directly or intermittently operate the restriction device, the energy transforming device may power the motor with the energy of the second form. Suitably, the restriction device is operable to perform a reversible function and the motor is capable of reversing said function.

In accordance with another embodiment of the invention, the restriction device comprises a hydraulic restriction device, and the apparatus comprises an implantable pump for operating the hydraulic restriction device, wherein the energy transforming device supplies the energy of the second form for driving the pump. Preferably, the pump is not a plunger type of pump, but may comprise a peristaltic or membrane pump.

The energy transforming device preferably is capable of generating as the energy of the second form a current exceeding 1 μA, when transferring the energy of the first form transmitted by the energy transmission device.

The apparatus may comprise an implantable adjustment device for adjusting the restriction device to change the restriction of the fecal passageway. In accordance with a first alternative the adjustment device is adapted to mechanically adjust the restriction device. In accordance with a second alternative the adjustment device is adapted to hydraulically adjust the restriction device by using implanted hydraulic means. Such hydraulic means may not use hydraulic fluid of the kind having a viscosity that substantially increases when exposed to heat or a magnetic field. The apparatus of the present invention is not limited to the use of visible light for the wireless transmission of energy. Thus, in accordance with a broad aspect of the invention, the energy transmission device transmits energy by at least one wireless signal, preferably containing radiant energy.

The wireless signal may comprises a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined. Alternatively, the wave signal may comprise a sound wave signal, such as an ultrasonic signal. Generally, the wireless signal may comprise a digital, analog or a digital and analog signal.

The energy of the first form transmitted by the energy transmission device may comprise an electric or magnetic field transmitted in pulses, for example digital pulses. Furthermore, the energy transforming device may transform the energy of the first form, which may comprise polarized energy, into a direct current, pulsating direct current, a combination of a direct and pulsating direct current, an alternating current or a combination of a direct and alternating current. Alternatively, the energy of the first form may comprise kinetic energy.

The energy of the second form may comprise a frequency, amplitude or frequency and amplitude modulated analog, digital or combined analog and digital signal. The restriction device may be non-inflatable, i.e. with no hydraulic fluid involved for the adjustments of the restriction device. This eliminates problems with fluid leaking from the restriction device.

The apparatus suitably comprises implantable electric conductors connected to the energy transforming device, whereby the energy transforming device is capable of supplying an electric current, such as direct current, a pulsating direct current, a combination of a direct and pulsating direct current, an alternating current or a combination of a direct and alternating current, via the conductors. Furthermore, the energy transforming device may be capable of supplying a frequency, amplitude, or frequency and amplitude modulated analog, digital, or a combination of analog and digital signal, which is used in connection with control of the restriction device.

In accordance with a main embodiment of the invention, the apparatus comprises an implantable operation device for operating the restriction device and a control device for controlling the operation device, wherein the energy transforming device powers the operation device with the energy of the second form. The operation device preferably comprises a motor, for example an electric linear motor or an electric rotary motor that is controlled by the control device to rotate a desired number of revolutions. Optionally, an implantable gearing may be connected to the motor. The electric motor may have electrically conductive parts made of plastics. Alternatively, the motor may comprise a hydraulic or pneumatic fluid motor, wherein the control device controls the fluid flow through the fluid motor. Motors currently available on the market are getting smaller and smaller. Furthermore, there is a great variety of control methods and miniaturized control equipment available. For example, the number of revolutions of a rotary motor may be analyzed by a Hall-element just a few mm in size. In accordance with another embodiment of the invention, the restriction device comprises hydraulic means and the operation device is adapted to conduct a hydraulic fluid in the hydraulic means. The operation device comprises a fluid conduit connected to the hydraulic means of the restriction device, and a reservoir for fluid, wherein the reservoir forms part of the conduit. The reservoir may form a fluid chamber with a variable volume, and the operation device may be adapted to distribute fluid from the chamber to the hydraulic means of the restriction device by reduction of the volume of the chamber and to withdraw fluid from the hydraulic means to the chamber by expansion of the volume of the chamber. The operation device suitably comprises an implantable motor used for reducing and expanding the volume of the chamber. Also, the operation device may comprise a pump for pumping the hydraulic fluid in the hydraulic means of the restriction device. All of the hydraulic components involved are preferably devoid of any non-return valve. This is of great advantage, because with valves involved there is always a risk of malfunction due to improperly working valves, especially when long time periods passes between valve operations.

The control device may be adapted to reverse the operation device by shifting polarity of the energy of the second form. Where the operation device comprises an electric motor the energy of the second form suitably comprises electric energy.

In accordance with yet another embodiment of the invention, the restriction device is operable to perform a reversible function, such as enlarging and restricting the blood flow passageway, and there is a reversing device implanted in the patient for reversing the function performed by the restriction device. Such a reversing function preferably involves enlarging and restricting the blood flow passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch it may be operable by the energy of the second form. In this case, the control device suitably controls the operation of the switch by shifting polarity of the energy of the second form supplied to the switch. The switch may comprise an electric switch and the source of energy may supply electric energy for the operation of the switch.

In accordance with an advantageous embodiment of the invention, the apparatus further comprises an energy storage device implanted in the patient for storing the energy of the second form and for supplying energy in connection with the operation of the restriction device. The implanted energy storage device preferably comprises an electric source of energy, such as an accumulator, a rechargeable battery or a combination of an accumulator and rechargeable battery.

The apparatus may further comprise a switch implantable in the patient for switching the operation of the restriction device and a source of energy implantable in the patient. This embodiment is particularly suited for applications where the energy transmission efficiency of the apparatus is insufficient, i.e. where the implanted restriction device is to perform more advanced operations. Such a source of energy preferably is a battery. Alternatively, the source of energy is an accumulator that also may store the energy of the second form.

In accordance with a first alternative, the switch is operated by the energy of the second form supplied by the energy storage device to switch from an off mode, in which the source of energy is not in use, to an on mode, in which the source of energy supplies energy for the operation of the restriction device. In this case, the implanted source of energy may comprise a battery, preferably having a lifetime of at least 10 years, or an accumulator. However, other kinds of sources are also conceivable, such as a nuclear source of energy or a chemical source of energy (fuel cells).

In accordance with a second alternative, the apparatus further comprises a remote control for controlling the supply of energy of the implanted source of energy, wherein the switch is operated by the energy of the second form supplied by the energy storage device to switch from an off mode, in which the remote control is prevented from controlling the source of energy and the source of energy is not in use, to a standby mode, in which the remote control is permitted to control the source of energy to supply energy for the operation of the restriction device.

In accordance with a third alternative, the energy storage device is omitted, wherein the switch is operated by the energy of the second form supplied by the energy transforming device to switch from an off mode, in which the remote control is prevented from controlling the source of energy and the source of energy is not in use, to a standby mode, in which the remote control is permitted to control the source of energy to supply energy for the operation of the restriction device. In accordance with a fourth alternative, also the remote control is omitted, wherein the switch is operated by the energy of the second form supplied by the energy transforming device to switch from an off mode, in which the source of energy is not in use, to an on mode, in which the source of energy supplies energy for the operation of the restriction device. Where applicable, in the described embodiments the switch may switch when the energy transmission device is transmitting wireless energy, preferably while the transferred energy of the second form is stabilized by an implanted capacitor, which may temporarily (for a few seconds) store the energy of the second form.

In the above noted third and fourth alternatives, the energy transmission device may be substituted for the energy transforming device, whereby the switch is operated by the energy of the first form.

The switch mentioned above may comprise an electronic switch or, where applicable, a mechanical switch.

The advantage of using a switch above all is increased control safety; i.e. interfering signals in the patient's surroundings cannot affect the implanted restriction device. Furthermore, the lifetime of the implanted source of energy will be significantly prolonged, since the energy consumption of the apparatus will be reduced to a minimum. During the above-mentioned standby mode, the remote control uses energy from the implanted source of energy. By means of the energy transmission device energy may be transmitted to activate the switch to connect the implanted source of energy only when energy is required in connection with the operation of the restriction device.

All of the above embodiments may be combined with at least one implantable sensor for sensing at least one physical parameter of the patient, wherein the control device may control the restriction device in response to signals from the sensor. Advantageously, the sensor may sense ejaculation, and the restriction device may release said tissue portion in response to the sensor sensing ejaculation.

The control device may comprise an internal control unit implantable in the patient for, preferably directly, controlling the restriction device in response to signals from the sensor. In response to signals from the sensor, for example signals related to pressure, the patient's position or any other important physical parameter, the internal control unit may send information thereon to outside the patient's body. The control device may also automatically control the restriction device in response to signals from the sensor. For example, the control device may control the restriction device to further restrict the blood flow passageway in response to the sensor sensing that the patients' blood flow and blood pressure are increasing, or enlarge the blood flow passageway in response to the sensor sensing an abnormally high pressure against the restriction device or sensing ejaculation.

Alternatively, the control device may comprise an external control unit outside the patient's body for, suitably directly, controlling the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor. An external data communicator may be provided outside the patient's body and an internal data communicator may be implanted in the patient for communicating with the external communicator. The internal communicator may feed data related to the patient, or related to the restriction device, back to the external communicator. Alternatively or in combination, the external communicator may feed data to the internal communicator. The internal communicator may suitably feed data related to at least one physical signal of the patient.

The apparatus may further comprise an implantable programmable control unit for controlling the restriction device, preferably over time in accordance with an activity schedule program. This will advance the apparatus and make possible an adaptation of the apparatus to the individual patients.

Many of the above embodiments are suitably remote controlled. Thus, the apparatus advantageously comprises a wireless remote control transmitting at least one wireless control signal for controlling the restriction device. With such a remote control it will be possible to adapt the function of the apparatus to the patient's need. The control signal may comprise a frequency, amplitude or frequency or amplitude modulated signal. Furthermore, the control signal may comprise an analog or a digital signal, or a combination of an analog and digital signal.

The wireless remote control may be capable of obtaining information on the condition of the implanted restriction device and of controlling the restriction device in response to the information. Also, The remote control may be capable of sending information related to the restriction device from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient. The wireless remote control may transmit a carrier signal for carrying the control signal, wherein the carrier signal is frequency, amplitude or frequency and amplitude modulated. The carrier signal may also comprise digital, analog or a combination of digital and analog signals. Such signals may comprise wave signals. Also the control signal used with the carrier signal may be frequency, amplitude or frequency and amplitude modulated, and be digital, analog or combined digital and analog.

The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control. The energy transmission device may function different from or similar to the energy transforming device. For example, the energy transmission and transforming devices function differently when the energy transmission device comprises a coil used for transmitting the energy of the first form and the energy transforming device comprises an electrical junction element for transforming the transmitted energy into the energy of the second form. The energy transmission and transforming devices function similar to each other when the energy transmission device comprises a coil used for transmitting the energy of the first form and the energy transforming device also comprises a coil for transforming the transmitted energy into the energy of the second form.

In accordance with an alternative embodiment of the invention, the apparatus comprises an activatable source of energy implantable in the patient, wherein the source of energy is activated by wireless energy transmitted by the energy transmission device, to supply energy which is used in connection with the operation of the restriction device.

The implantable restriction device suitably is embedded in a soft or gel-like material. For example, a silicone material having hardness less than 20 Shore.

All the above described various components, such as the motor, pump and capacitor, may be combined in the different embodiments where applicable. Also the various functions described in connection with the above embodiments may be used in different applications, where applicable.

All the various ways of transferring, transforming and controlling energy presented in the present specification may be practiced by using all of the various components and solutions described.

The present invention also provides methods for implanting the apparatus of the invention, and for treating impotence.

Accordingly, there is provided an implanting method, comprising the steps of: providing an impotence treatment apparatus as described above, cutting an opening in a patient's mucosa in the orifice of the patient's body, and implanting the energy transforming means in the patient's body through the opening. Alternatively, the cutting step may comprise cutting the opening in the patient's skin and the implanting step may comprise implanting the energy transforming means in the patient's body through the opening.

There is also provided a laparascopical implanting method, comprising the steps of: providing the impotence treatment apparatus as described above, placing at least two laparascopic cannula within a patient's body, and implanting the energy transforming means in the patient's body by using the at least two laparascopic cannula.

In accordance with another alternative there is provided a laparascopical surgical method of implanting the impotence treatment apparatus, comprising the steps of: a) Providing the impotence treatment apparatus as described above, b) Placing at least two laparoscopic trocars within the patient's body, c) Using a dissecting tool inserted through the laparoscopic trocars, dissecting the region of the penile tissue or the prolongation thereof, d) Introducing the operable restriction device of the apparatus through the trocars, e) Placing the restriction device in engagement with the penile tissue or the prolongation thereof to create a stoma through which blood flow leaves the penis. And f) implanting the energy transforming means of the apparatus in the patient for transforming wireless energy into energy of a form suited for operating the restriction device to reduce the stoma to restrict the blood flow therethrough. The method as recited in (a)-(f) may further comprise adjusting the reduced stoma in a non-invasive procedure.

There is also provided a method of treating an impotent male comprising: (a) Surgically implanting in a male patient a restriction device engaging the male's penile tissue or the prolongation thereof to form a restrictable passageway in which blood flow leaves the penis, (b) Surgically implanting in the male the operation device which can adjust the restricted passageway in response to supplied energy. And (c) in a non-invasive post-operative procedure, from time to time, supplying energy to the operation device so as to restrict the passageway to reduce the blood flow leaving the male's penis to achieve erection. In the method (c) may be practiced by the patient whenever he desires.

### AA: Male Sexual Impotence Treatment Apparatus

The present aspect relates to a male sexual impotence treatment apparatus comprising an adjustable restriction device implantable in a male impotent patient for engaging the corpora cavernosa or crura or the prolongations thereof of the patient's penile tissue. An implantable adjustment device is provided for adjusting the restriction device to temporarily contract the corpora cavernosa or crura or the prolongations thereof to restrict the blood flow leaving the penis, and an implantable operation device is provided for operating the adjustment device. Male sexual impotence is a widespread problem. Many different solutions to this problem have been tried. A main solution currently practiced and disclosed in for instance U.S. Pat. Nos. 5,437,605 and 4,841,461 is to implant a hydraulic inflatable/contractable silicon prosthesis in the cavities of the corpora cavernosa of the patient's penis. In fluid connection with this prosthesis is a reservoir implanted in the scrotum. By manual pumping action the prosthesis is filled with fluid from the reservoir to effect erect penile condition or is emptied of fluid, which returns to the reservoir, to effect flaccid penile condition. However, there are several more or less severe disadvantages of this main solution. Above all, the penis is more or less damaged by the operation and it is practically impossible to reverse the operation. Another disadvantage is that rather strong forces act against this implanted prosthesis resulting in a significant risk of the prosthesis being broken.

Another solution to achieve erection is to restrict the blood flow leaving the penis. For example, U.S. Pat. No. 4,829,990 discloses two hydraulically operated inflatable cuffs wrapped around the respective crura. A disadvantage of such a solution is that it involves complicated surgery. Another example on this solution is U.S. Pat. No. 4,828,544, which discloses an artificial fistula system surgically implanted and providing a primary fistula between the femoral artery and the femoral vein and a secondary fistula for leading blood from the primary fistula to the penis. An inflatable balloon engages the primary fistula between the secondary fistula and the vein. The balloon is in fluid connection with a manually compressible reservoir implanted in the scrotum. Again, implantation of this artificial fistula system requires delicate surgery.

Yet another solution is to inject a substance in the penile vein system to achieve erection.

However, injections are painful and complicated for the patient.

The object of the present invention is to provide a simple male sexual impotence treatment apparatus, which requires relatively uncomplicated surgery.

This object is obtained by an apparatus of the kind described initially characterized in that the operation device comprises a powered operation device.

With the expression “the prolongations thereof” should be understood the penile tissue extending from the crura inside the human body and following the pathway of the blood flow leaving the penis, i.e. where the penile exit veins extend.

Generally, the restriction device is adapted to contract the corpora cavernosa or crura or the prolongations thereof as a single unit, which requires relatively simple surgery.

Alternatively, however, the restriction device may comprise two restriction members adapted to contract the respective corpora cavernosa or crura or the prolongations thereof.

The restriction device may control the contraction of the corpora cavernosa or crura or the prolongations thereof, preferably to change steplessly. Suitably, the adjustment device adjusts the restriction device such that the restriction device provides a predetermined contraction of the corpora cavernosa or crura or the prolongations thereof that is satisfactory for the patient. A satisfactory contraction is achieved when the differential pressure between the arterial and venous penile blood pressures has increased enough to cause erection of the patient's penis while blood still is flowing through the penis.

The adjustment device preferably adjusts the restriction device in a non-invasive manner. The adjustment device may adjust the restriction device in a non-magnetic manner, i.e. magnetic forces may not be involved when adjusting the restriction device. Also, the adjustment device may adjust the restriction device in a non-thermal manner, i.e. thermal energy may not be involved when adjusting the restriction device.

The expression “powered” should be understood as energized with everything without manual force, preferably electric energy. In other words, the adjustment device is operated in a non-manual manner. The expression “non-manual manner” should be understood to mean that the adjustment device is not operated by manually touching subcutaneously implanted components of the apparatus or not manipulated by touching the skin of the patient. Thus, as opposed to prior practice when treating male sexual impotence, the adjustment device is not operated by manual forces, such as by manually compressing a fluid containing balloon implanted in the scrotum or in the region of labia majora. Of course, manual manipulation of a subcutaneous start button or the like for activating the powered operation device is permitted within the scope of the present invention.

Alternatively, or in combination with a powered operation device, the servo means may be used, which enables manual manipulation without need for strong manipulation forces. The servo means may comprise hydraulic means, electric control means, magnetic means, or mechanical means, which may be activated by manual manipulating means. Using a servo system will save the use of force when adjusting the adjustment device, which may be of importance in many applications.

The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible.

In accordance with a main embodiment of the invention, the apparatus comprises a reservoir, preferably containing a predetermined amount of hydraulic fluid, also implantable in the patient, wherein the operation device, suitably electrically powered, operates the adjustment device by using the hydraulic fluid of the reservoir.

The adjustment device may comprise an expandable cavity in the restriction device, wherein the corpus cavernosum or crura or the prolongation thereof is squeezed upon expansion of the cavity and released upon contraction of the cavity. In this embodiment the operation device is adapted to distribute hydraulic fluid from the reservoir to expand the cavity, and from the cavity to the reservoir to contract the cavity.

A fluid distribution tube may readily be connected between the reservoir and the cavity in a manner so that the tube does not interfere with other implanted components of the apparatus.

Preferably, the reservoir defines a chamber for the predetermined amount of fluid and the operation device changes the volume of the chamber. The operation device suitably comprises first and second wall portions of the reservoir and is adapted to provide relative displacement between the first and second wall portions of the reservoir, in order to change the volume of the chamber.

The operation device may be adapted to provide said relative displacement in response to the pressure in the reservoir. Suitably, the operation device comprises a pressure controlled hydraulic operation device. For safety, an alarm may be provided for generating an alarm signal in response to the lapse of a predetermined time period during which the pressure controlling the hydraulic operation device exceeds a predetermined high value.

Suitably, the operation device is adapted to distribute fluid from the reservoir to the cavity of the restriction member in response to a predetermined first displacement of the first wall portion of the reservoir relative to the second wall portion of the reservoir and may distribute fluid from the cavity to the reservoir in response to a predetermined second displacement of the first wall portion relative to the second wall portion.

The first and second wall portions of the reservoir may be displaceable relative to each other by a magnetic, hydraulic, or electric power means, such as an electric motor. In this embodiment no pump is used, only the volume of the e reservoir is varied. This is of great advantage compared to the solution described below when the operation device comprises a pump used to pump fluid between the reservoir and the adjustment device because there is no need for a non-return valve and it is still possible to have fluid going both to and from the reservoir. Thus, the significant risk of malfunction when using such a non-return valve implanted in the patient is eliminated.

The operation device may comprise hydraulic means and a fluid conduit extending between the hydraulic means and the adjustment device. The hydraulic means and conduit are devoid of any non-return valve. The reservoir may form part of the conduit and a fluid chamber with a variable volume. The operation device may distribute fluid from the fluid chamber to the adjustment device by reduction of the volume of the chamber and withdraw fluid from the adjustment device by expansion of the volume of the chamber. The operation device preferably comprises a motor for moving a movable wall of the reservoir for changing the volume of the chamber. Any kind of motor could be used for the different operations as well as wireless remote solutions for controlling the operations.

The restriction device preferably is operable to perform a reversible function and accordingly there is a reversing device implantable in the patient for reversing the function performed by the restriction device. Such a reversing function preferably involves enlarging and restricting the blood flow passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch. Where the operation device comprises a motor, the reversing device is adapted to reverse the motor.

In accordance with another particular embodiment of the invention, the operation device comprises a pump for pumping fluid between the reservoir and the adjustment device. A mechanical solution is proposed in which it is possible to pump fluid from the reservoir to the adjustment device and vice versajust by pushing an activation member in one direction. The pump preferably comprises a first activation member for activating the pump to pump fluid from the reservoir to the adjustment device, and a second activation member for activating the pump to pump fluid from the adjustment device to the reservoir. At least one of the first and second activation members may be operable by manual manipulation, preferably to permit manual pushing, pulling or rotation thereof in one direction, or by a device powered magnetically, hydraulically, or electrically (e.g. by an electric motor), or be operable by a combination of these methods. Suitably, at least one of the activation members may be adapted to operate when subjected to an external pressure exceeding a predetermined magnitude. Another alternative is a pump pumping in only one direction and an adjustable valve to change the direction of fluid to either increase or decrease the amount of fluid in the reservoir. This valve may be manipulated either manually, mechanically, magnetically, or hydraulically.

The main embodiment described above including the reservoir may alternatively be equipped with a servo means comprising a reverse servo. The term “reverse servo” is to be understood as a mechanism that transfers a strong force acting on a moving element having a short stroke into a weak force acting on another moving element having a long stroke; i.e. the reverse function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device.

The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the normal servo means and the specific reverse servo may be used in connection with all of the various components and solutions described in the present specification.

Thus, the reverse servo may be adapted to provide relative displacement between the first and second wall portions of the reservoir, suitably in response to the pressure in the reservoir, in order to change the volume of the chamber of the reservoir.

Generally, the servo means, including the reverse servo, comprises a pressure controlled servo means. The alarm mentioned above may alternatively be adapted to generate an alarm signal in response to the lapse of a predetermined time period during which the pressure controlling the servo means exceeds a predetermined high value. The reverse servo may comprise magnetic means, electric means or manual manipulation means or a combination thereof. Preferably, however, the reverse servo comprises hydraulic means.

In accordance with a particular embodiment of the invention, the reverse servo further comprises a servo reservoir defining a chamber containing servo fluid, and the operation device comprise first and second wall portions of the servo reservoir, which are displaceable relative to each other to change the volume of the chamber of the servo reservoir. The first and second wall portions of the servo reservoir may be displaceable relative to each other by magnetic means, hydraulic means, or electric control means.

Where the reverse servo comprises hydraulic means it may further comprise a fluid supply reservoir connected to the servo reservoir in a closed system and containing a further predetermined amount of fluid. The fluid supply reservoir defines a chamber for the further predetermined amount of fluid and the operation device is adapted to change the volume of the chamber and thereby control the amount of fluid in the servo reservoir. The fluid supply reservoir comprises first and second wall portions, which are displaceable relative to each other to change the volume of the chamber of the fluid supply reservoir. Suitably, the fluid supply reservoir increases the amount of fluid in the servo reservoir in response to a predetermined first displacement of the first wall portion of the fluid supply reservoir relative to the second wall portion of the fluid supply reservoir and decreases the amount of fluid in the servo reservoir in response to a predetermined second displacement of the first wall portion of the fluid supply reservoir relative to the second wall portion of the fluid supply reservoir. In accordance with an embodiment of the invention, the adjustment device comprises a hydraulic adjustment device, and an implantable reservoir containing a predetermined amount of hydraulic fluid and a conduit providing fluid connection between the reservoir and the hydraulic adjustment device are provided. The operation device is adapted to operate the hydraulic adjustment device by distributing hydraulic fluid through the conduit between the reservoir and the hydraulic adjustment device, wherein the conduit and hydraulic adjustment device are devoid of any non-return valve to permit free flow of hydraulic fluid in both directions in the conduit. Preferably, the reservoir forms a fluid chamber with a variable volume, and the operation device is adapted to distribute fluid from the chamber to the adjustment device by reduction of the volume of the chamber and to withdraw fluid from the adjustment device by expansion of the volume of the chamber. The operation device may comprise a motor or a pump. Alternatively, the operation device may comprise a movable wall of the reservoir for changing the volume of the chamber. For example, the operation device may be adapted to change the volume of the chamber by moving the movable wall in response to the pressure in the chamber. In the above embodiments including a reservoir for hydraulic fluid an injection port may be provided for subcutaneous implantation in the patient to be in fluid communication with the chamber of the reservoir. The injection port may be integrated in the reservoir. Such an injection port may be provided for enabling, normally single, once-and-for-all, calibration of the amount of fluid in the hydraulic system used.

In the various embodiments hereinafter described the restriction device generally forms an at least substantially closed loop. However, the restriction device may take a variety of different shapes, such as the shape of a square, rectangle or ellipse. The substantially closed loop could for example be totally flat, i.e. thin as seen in the radial direction. The shape of restriction device may also be changed during use, by rotation or movements of the restriction device in any direction. A physical lumen, such as the corpora cavernosa or crura, often is easier to contract by moving two opposite sidewalls of the lumen against each other. Thus, the restriction device may be designed to perform such a contracting effect of the opposite walls of the corpora cavernosa or crura or the prolongations thereof, or alternatively the opposite walls of each one of the corpora cavernosa or crura or the prolongations thereof. Either mechanical or hydraulic solutions may be employed to operate the restriction device. Alternatively, the restriction device may comprise an adjustable cuff, a clamp or a roller for bending or rotating the corpora cavernosa or crura or the prolongations thereof to contract them. The bending or rotating members may take any shape and be either hydraulic or non-inflatable.

Preferably the restriction device comprises an elongated restriction member and forming means for forming the restriction member into at least a substantially closed loop around the corpora cavernosa or crura or the prolongations thereof, wherein the loop defines a restriction opening, whereby the adjustment device adjusts the restriction member in the loop to restrict the blood flow leaving the penis.

Advantageously, the forming means may form the restriction member into a loop having a predetermined size. Alternatively, the forming means may form the restriction member into a loop selected from several predetermined sizes. The adjustment device may change the size of the restriction opening such that the outer circumferential confinement surface of the restriction member either is changed or is unchanged.

The elongated restriction member may be flexible, for example take the shape of a belt or cord, and the adjustment device may pull a first portion of the flexible restriction member from a second portion of the flexible restriction member opposite the first portion in the loop to squeeze the corpora cavernosa or crura or the prolongations thereof between the opposite lengths of the elongated flexible restriction member to restrict the blood flow leaving the penis. The restriction member may be non-inflatable, and the adjustment device may mechanically adjust the restriction member in the loop.

Preferably the adjustment device is operable to adjust the restriction device to steplessly change the contraction of the corpora cavernosa or crura or the prolongations thereof.

The adjustment device may mechanically or hydraulically adjust the restriction device. In the embodiments described the adjustment device may either mechanically or hydraulically adjust the restriction device, where applicable. It should be noted that the operation device might mechanically or hydraulically operate the adjustment device irrespectively of whether the adjustment device is adapted to adjust the restriction device mechanically or hydraulically.

In accordance with an embodiment of the invention, the restriction device comprises at least two elements on opposite or different sides of the corpus cavernosum or crura or the prolongation thereof, and the adjustment device decreases the distance between the elements to squeeze the corpus cavernosum or crura or the prolongation thereof between the elements, thereby restricting the blood flow passageway. It is also possible to use only one element and squeeze the corpus cavernosum or crura or the prolongation thereof against human bone or tissue. The elements above may as well as all the restriction members mentioned in this application be everything from rigid to soft. In accordance with an alternative, the restriction device bends or rotates a portion of the corpus cavernosum or crura or the prolongation thereof to restrict the blood flow passageway in the same. For example, the restriction device may comprise at least two bending members, such as cylindrical or hour-glass shaped rollers, positioned on opposite or different sides of the corpus cavernosum or crura or the prolongation thereof and displaced relative to each other along the corpus cavernosum or crura or the prolongation thereof, and the adjustment device may move the bending members against the corpus cavernosum or crura or the prolongation thereof to bend the latter to restrict the blood flow passageway. The restriction device may also rotate a portion of the corpus cavernosum or crura or the prolongation thereof. The bending or rotating members may take any shape and be either hydraulic or non-inflatable.

Alternatively, the two bending members one placed more distal than the other may be rotated in opposite directions relative to each other. With interconnecting means for example flexible bands between the bending members a restriction will occur between the bending members when they are rotated.

Preferably the adjustment device is operable to adjust the restriction device to steplessly change the restriction of the blood flow passageway in the corpus cavernosum or crura or the prolongation thereof.

All embodiments according to the invention may be controlled by a wireless remote control.

In accordance with an advantageous embodiment of the invention, there is provided a wireless remote control for non-invasively controlling the operation device. The remote control may conveniently comprise an external hand-held remote control unit, which is manually operable by the patient to control the restriction device to squeeze and release the corpus cavernosum or crura or the prolongation thereof. With the wireless remote control the apparatus is conveniently controlled by the patient when he so desires, which is of great advantage compared to the prior art procedures. With the remote control the apparatus is conveniently controlled to adjust the implanted restriction device to release the blood flow passageway when the patient wants to relieve himself or herself.

The remote control may advantageously be capable of obtaining information related to important parameters, such as the condition of the blood flow passageway or the pressure against the restriction device, and of commanding the operation device to operate the adjustment device to adjust the restriction device in response to obtained information. With the remote control the apparatus is conveniently controlled to adjust the implanted restriction device to open and close the blood flow passageway. The adjustment device may control the restriction device to steplessly change the restriction of the passageway.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter. Any known or conventional signal transmitting or signal receiving means that is suitable for use with a human or mammal patient may be provided as the signal transmitter or signal receiver.

The apparatus may further comprise an implanted energizer unit for providing energy to energy consuming implanted components of the apparatus, such as electronic circuits and/or a motor for operating the adjustment device. Where a motor is provided the control unit is adapted to power the motor with energy provided by the energizer unit in response to a control signal received from the signal transmitter. The motor may be any type of motor, such as a pneumatic, hydraulic or electric motor and the energizer unit may power the motor with pressurized gas or liquid, or electric energy, depending on the type of motor. Where the motor is an electric motor, it may power pneumatic or hydraulic equipment.

The remote control advantageously comprises wireless energy transfer device for transferring energy from outside the patient's body to energy consuming implantable components of the apparatus. The energy transfer device may comprise said energizer unit is adapted to transform energy from the control signal, as it is transmitted to the signal receiver, into electric energy. Where the operation device comprises a motor the wireless energy transfer device is adapted to directly power the motor with transferred energy.

The energy transferred by the wireless energy transfer device preferably comprises a signal, suitably a wave signal. The energy transferred by the wireless energy transfer device may comprise an electric field or a magnetic field or a combination thereof. The signal may be analog or digital or a combination thereof. The energy transfer device may transfer the energy from the signal into a direct, pulsating direct or alternating current or a combination thereof.

Any of the above mentioned signals may comprise analog or digital pulses. The analog or digital signal may comprise a magnetic field or an electric field or a combination thereof. Where the signal is a wave signal it may comprise an electromagnetic wave signal, a sound wave signal or a cater wave signal for a remote control signal or a combination thereof. Where a carrier signal is used it may be frequency, amplitude or frequency and amplitude modulated.

The apparatus may comprise an implantable source of energy for powering the operation device and/or for energizing other energy consuming components of the apparatus, wherein the energy from the source of energy is releasable from outside the patient's body. Furthermore, the apparatus may comprise an energy transmission device for wireless transmission of energy of a first form and an energy transforming device implantable in the patient for transforming the energy of the first form into energy of a second form, to be supplied to the source of energy and/or other implantable energy consuming parts of the apparatus. The energy transforming device may transform the wireless energy directly or indirectly into energy different than the wireless energy for operation of the restriction device. Typically, the energy of the second form is different than the energy of the first form. The function of the energy transmission device may be different from that of the energy transforming device.

An implantable motor or pump for operating the adjustment device may be provided, wherein the energy transmission device may be adapted to transmit wireless energy in the form of a magnetic field or electromagnetic waves or field for direct power of the motor or pump, as the wireless energy is being transmitted. Suitably, the energy transmission device transmits energy by at least one signal separate from the above mentioned control signal. An implantable stabilizer for stabilizing the energy of the first or second form may be provided. Where the energy of the second form comprises electric current, the stabilizer suitably comprises at least one capacitor.

Generally, the source of energy comprises a battery, accumulator, capacitor or a combination thereof.

In accordance with an embodiment of the invention, the apparatus comprises a control device adapted to produce wireless energy for directly powering the operation device and/or for energizing other energy consuming components of the apparatus.

Generally, the wireless energy may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal. Any of the above mentioned signals may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal.

The control device may be adapted to produce wireless energy in the form of a train of energy pulses and the energy transfer device may be adapted to intermittently transfer the train of energy pulses for direct use in connection with the energizing of the energy consuming components of the apparatus. Alternatively, the control device may be adapted to control the energy transforming device to produce the energy of the second form in said train of energy pulses for direct use in connection with the operation of the adjustment device. The transferred energy preferably comprises electric energy. An implantable capacitor may be provided for producing the train of energy pulses.

Where a capacitor is used in any of the above described embodiments it may have a relatively low capacity, i.e. less than 0.1 μF, in order to be small and suited for implantation.

Where the operation device comprises an implantable motor or pump for operating the adjustment device, the energy transfer device may be adapted to directly power the motor or pump with transferred energy, at the same time as the energy is transferred. Where a pump is used it should not be a plunger type of pump, because a plunger pump is noisy, but may comprise a peristaltic or membrane pump. As mentioned above the apparatus comprises a wireless remote control for non-invasively controlling the operation device, which preferably is electrically powered. Alternatively, the operation device is powered by magnetic energy, non-magnetic energy, electromagnetic energy, non-electromagnetic energy, kinetic energy, non-kinetic energy, sonic energy, non-sonic energy, thermal energy or non-thermal energy. However, the operation device may be unpowerable by permanent static magnetic energy. Any other kind of energy, such as electric, electromagnetic energy or a moving permanent magnetic energy, may be conceivable for operating the adjustment device. As a result, the implanted restriction device would not be accidentally adjusted if the patient comes close to any permanent magnet. Suitably, the operation device is adapted to non-invasively operate the adjustment device.

Where the operation device comprises a hydraulic operation device it may use hydraulic fluid, the viscosity of which changes when the hydraulic fluid is exposed to energy, preferably electric energy, different than thermal energy. However, use of hydraulic fluid of the kind having a viscosity that substantially increases when exposed to heat or a magnetic field, i.e. the hydraulic fluid would not become more viscous when exposed to heat or influenced by magnetic forces, should be avoided, because external heat sources or heat from the body when the patient has fever and external magnetic sources might affect the implanted components of the apparatus.

The adjustment device is may be operable to adjust the restriction device to steplessly change the restriction of the blood flow passageway. Furthermore, the adjustment device may be adapted to mechanically adjust the restriction device. Alternatively, it may be adapted to hydraulically adjust the restriction device by using hydraulic means, which is devoid of hydraulic fluid of the kind having a viscosity that substantially increases when exposed to heat or a magnetic field.

In accordance with an embodiment of the invention, the apparatus comprises a control device for controlling the restriction device. The control device may comprise an internal programmable control unit implantable in the patient and, possibly an external control unit outside the patient's body for programming the programmable internal control unit. Alternatively, the external control unit may be programmable and wirelessly control the restriction device. The control device may be adapted to produce wireless energy for directly powering the operation device and/or for energizing other energy consuming components of the apparatus. At least one sensor for sensing at least one physical parameter of the patient may conveniently be implanted in the patient. The sensor may preferably sense as the physical parameter ejaculation, for example by sensing the pressure or flow in the patient's urethra, and either the internal control unit or the external control unit of the control device may suitably control the restriction device to release the corpora cavernosa or crura or the prolongations thereof in response to the sensor sensing ejaculation. For safety the restriction device may release the corpora cavernosa or crura or the prolongations thereof in response to the sensor sensing for example an abnormally high pressure value. The internal control unit may directly control the restriction device in response to signals from the sensor.

The pressure sensor may be any suitable known or conventional pressure sensor such as shown in U.S. patents 5540731, 4846181, 4738267, 4571749, 4407296 or 3939823; or an NPC-102 Medical Angioplasty Sensor. Either the internal control unit or the external control unit of the control device may suitably control the restriction device to enlarge or close the blood flow passageway. For safety the restriction device may enlarge or open the blood flow passageway in response to the sensor sensing for example an abnormally high pressure value. The internal control unit may directly control the restriction device in response to signals from the sensor.

Wherever magnetic means is utilized according to the invention it may comprise a permanent magnet and a magnetic material reed switch, or other suitable known or conventional magnetic means.

Where a source of energy is used the control device suitably is operable from outside the patient's body for controlling the source of energy to release energy for use in connection with the operation of the adjustment device, when the adjustment device is implanted. The source of energy may be provided external to the patient's body, and the control device may be adapted to control the external source of energy to release wireless energy for use in connection with the operation of the adjustment device.

The control device may control the source of energy to release magnetic energy, non-magnetic energy, electromagnetic energy, non-electromagnetic energy, kinetic energy, non-kinetic energy, sonic energy, non-sonic energy, thermal energy or non-thermal energy, preferably in a non-invasive manner and for a determined time period and/or in a determined number of energy pulses.

Where the implantable components of the apparatus comprise electrical components they may include at least one or a single voltage level guard. In this case, the electrical components suitably are devoid of any current detector and/or charge level detector. Furthermore, the electrical components may comprise a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

In accordance with an advantageous embodiment of the invention, the apparatus comprises an implantable switch for directly or indirectly switching the operation of the restriction device. The switch may be operated by the energy supplied by the energy transmission device mentioned above to switch from an off mode, in which the implantable source of energy mentioned above is not in use, to an on mode, in which the source of energy supplies energy for the operation of the restriction device.

In accordance with an alternative embodiment, the above mentioned a remote control may be employed for controlling the implantable source of energy, wherein the switch is operated by the energy supplied by the energy transmission device to switch from an off mode, in which the remote control is prevented from controlling the source of energy and the source of energy is not in use, to a standby mode, in which the remote control is permitted to control the source of energy to supply energy for the operation of the restriction device.

In accordance with another alternative embodiment, the switch is operated by the energy supplied by the implantable energy transforming device mentioned above to switch from an off mode, in which the source of energy is not in use, to an on mode, in which the source of energy supplies energy for the operation of the restriction device.

In accordance with yet another alternative embodiment, the switch is operated by the energy supplied by the energy transforming device to switch from an off mode, in which the remote control is prevented from controlling the source of energy and the source of energy is not in use, to a standby mode, in which the remote control is permitted to control the source of energy to supply energy for the operation of the restriction device.

Suitably, the restriction device is embedded in a soft or gel-like material, such as a silicone material having hardness less than 20 Shore.

The energy transforming device may be designed to be implanted subcutaneously or in the abdomen, thorax or cephalic region of the patient.

The adjustment device may be adapted to adjust the restriction device such that the restriction device provides a predetermined contraction of the blood flow passageway that is satisfactory for the patient.

All the above described various components, such as the motor, pump and capacitor, may be combined in the different embodiments where applicable. Also the various functions described in connection with the above embodiments may be used in different applications, where applicable. Specifically, the various remote control functions described and all the various methods for supplying energy may be used in any conceivable combination that is apparent to those skilled in the art.

The invention also provides a method for treating male sexual impotence, comprising surgically implanting in the body of a male impotent patient an adjustable restriction device engaging the corpora cavernosa or crura or the prolongations thereof as a single unit, and when desired to achieve erection, adjusting the restriction device to temporarily contract the corpora cavernosa or crura or the prolongations thereof to restrict the blood flow leaving the penis.

The invention also provides another method for treating male sexual impotence, comprising surgically implanting in the body of a male impotent patient at least two adjustable restriction devices engaging the respective corpora cavernosa or crura or the prolongations thereof as separate units of the patients penis, and when desired to achieve erection, non-manually without touching the skin of the patient adjusting the restriction devices to temporarily contract the corpora cavernosa or crura or the prolongations thereof to restrict the blood flow leaving the penis. The method may further comprise surgically implanting one or more non-manually adjustable powered restriction devices engaging respective exit veins from the penis.

The method may further comprise implanting a source of energy in the patient and providing a control device for controlling the source of energy from outside the patient's body to supply energy to the restriction device or alternatively energizing energy consuming components of the apparatus with wirelessly transmitted energy from outside the patient's body.

The invention also provides a laparascopic surgical method. Thus, there is provided a method comprising the steps of placing at least two laparascopical trocars in a male impotent patient's body, inserting a dissecting tool through the trocars and dissecting an area of the penis, abdominal or pelvic or retroperitoneal surroundings, and placing an adjustable restriction device in the dissected area, so that the restriction device engages the corpora cavernosa or crura or the prolongations thereof.

The method may further comprise hydraulically adjusting the restriction device in a non-manual manner. The restriction device may engage (a) both of the corpora cavernosa or crura or the prolongations thereof as a single unit; or (b) one of the exit veins from the penis. Alternatively, the method may further comprise implanting (a) a further adjustable restriction device, wherein the two restriction devices engage the two corpora cavernosa or crura of the penis or their prolongations, respectively, as separate units; or (b) several restriction devices engaging respective exit veins from the penis.

The method may further comprise implanting a source of energy in the patient and providing a control device for controlling the source of energy from outside the patient's body to supply energy to the restriction device, and optionally also to other energy consuming components of the apparatus, with wirelessly transmitted energy from outside the patient's body.

The operation device may preferably be powered with electricity and operated in a non-manual manner.

### AB: Impotence Treatment Apparatus

The present aspect relates to a male sexual impotence treatment apparatus, comprising an adjustable restriction device implantable in a male patient, who suffers from sexual impotence, for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection.

Male sexual impotence is a widespread problem. Many different solutions to this problem have been tried. A main solution currently practiced and disclosed in for instance U.S. Pat. Nos. 5,437,605 and 4,841,461 is to implant a hydraulic inflatable/contractible silicon prosthesis in the cavities of the corpora cavernosa of the patient's penis. In fluid connection with this prosthesis is a reservoir implanted in the scrotum. By manual pumping action the prosthesis is filled with fluid from the reservoir to effect erect condition or is emptied of fluid, which returns to the reservoir, to effect flaccid condition. However, there are several more or less severe disadvantages of this main solution. Above all, the penis is more or less damaged by the operation and it is practically impossible to reverse the operation. Another disadvantage is that rather strong forces act against this implanted prosthesis resulting in a significant risk of the prosthesis being broken. A further disadvantage is that hard fibrosis created around the reservoir over time may cause malfunction of pumping components. Thus, the created fibrosis will sooner or later become a hard fibrotic layer which may make it difficult to pump the reservoir. Yet a further disadvantage is that the use of hydraulic fluid always entails a risk of fluid leaking from the prosthesis. Furthermore, it is a rather complicated task to manually pump the reservoir when erection is desired.

Another solution to achieve erection is to restrict the blood flow leaving the penis. For example, U.S. Pat. No. 4,829,990 discloses two hydraulically operated inflatable cuffs wrapped around the respective crura. Again, a disadvantage of such a solution is that it entails a risk of hydraulic fluid leaking from the cuffs.

Another example of the solution to restrict the penile blood flow is found in U.S. Pat. No. 4,828,544, which discloses an artificial fistula system surgically implanted and providing a primary fistula between the femoral artery and the femoral vein and a secondary fistula for leading blood from the primary fistula to the penis. An inflatable balloon engages the primary fistula between the secondary fistula and the vein. The balloon is in fluid connection with a manually compressible reservoir implanted in the scrotum. Besides the risk of fluid leaking from the balloon, a further disadvantage of this latter example is that it requires delicate surgery.

A main disadvantage of the solution to restrict the penile blood flow is the fact that the venous blood vessel system is rather complex and it is difficult to restrict the veinplexa. Yet another solution is to inject a substance in the vein system to achieve erection. However, injections are painful and complicated for the patient. The prime object of the present invention is to provide a male sexual impotence treatment apparatus in which the risk of liquid leaking from implanted hydraulic components of the apparatus is substantially reduced or completely eliminated.

A further object is to provide a male sexual impotence treatment apparatus, which does not require surgical implantation of any fistula system.

Another object is to provide a male sexual impotence treatment apparatus, which does not require the use of an injection needle.

These objects are obtained by an apparatus of the kind stated initially, which is characterized in that the adjustment device is adapted to mechanically adjust the restriction device.

The restriction device preferably is non-inflatable.

Preferably, the adjustment device adjusts the restriction device in a non-invasive manner.

Furthermore, the adjustment device may adjust the restriction device in a non-manual manner and/or in a non-magnetic manner, i.e. magnetic forces may not be involved when adjusting the restriction device.

Furthermore, as opposed to prior art impotence treatment devices the adjustment device preferably is non-manually operated, i.e. not operated by manual forces or manipulated by touching the skin of the patient, such as by manually compressing a fluid containing balloon implanted in the scrotum.

Instead the apparatus may further comprise a powered operation device for operating the adjustment device. In the various embodiments hereinafter described the restriction device generally forms an at least substantially closed loop. However, the restriction device may take a variety of different shapes, such as the shape of a square, rectangle or ellipse. The substantially closed loop could for example be totally flat, i.e. thin as seen in the radial direction. The shape of restriction device may also be changed during use, by rotation or movements of the restriction device in any direction.

A physical lumen, such as the corpus cavernosum, crura of the penile tissue or the prolongation thereof, is often easier to restrict by contracting at least two opposite or different side walls of the lumen against each other. The expression “penile tissue and the prolongation thereof” should be understood to mean the penile tissue extended inside the human body and following the pathway of the blood flow leaving the penis i.e. one or several exit veins from the penis, corpus cavernosum, crura or the prolongation thereof. Thus, the restriction device may be designed to perform such a contracting effect of the opposite walls also of an exit vein in the penile prolongation.

Alternatively, the restriction device may comprise an adjustable cuff, a clamp or a roller for bending the vein, corpus cavernosum, crura or the prolongation thereof to restrict the blood flow therein. Such a cuff, clamp or roller may also be utilized for squeezing the vein, corpus cavernosum, crura or the prolongation thereof against human material inside the body of the patient or against implanted structures of the apparatus.

Preferably, the restriction device comprises an elongated restriction member and forming means. for forming the restriction member into at least a substantially closed loop around said portion of the tissue, wherein the loop defines a restriction opening, whereby the adjustment device adjusts the restriction member in the loop to change the size of the restriction opening.

The restriction device may be implanted in the base of the patient's penis or the prolongation thereof and preferably may engage the corpus cavernosum, crura or the prolongation thereof of the penis. However, there are several alternative positions of the restriction device that give more or less satisfactory restriction of the blood flow leaving the penis. Thus, as a first alternative the restriction member may extend around both corpora cavernosa or crura of the penis as a single unit. As a second alternative the restriction device may comprise two elongated restriction members extending around respective corpora cavernosa or crura of the patient. As a third alternative the elongated restriction member may encircle one of the exit veins from the penis. As a fourth alternative the restriction device may comprise several restriction members extending around respective exit veins from the penis.

The adjustment device may be incorporated in the restriction device as well as be controlled by hydraulic means.

In accordance with a preferred first adjustment principle, the adjustment device mechanically adjusts the longitudinal extension of the elongated restriction member in a loop form. In a preferred embodiment utilizing the first adjustment principle, the restriction member comprises a main portion and two elongated end portions, and the adjustment device establishes longitudinal relative, displacement between the end portions of the restriction member, so that the size of the restriction opening is adjusted. The forming means may comprise any suitable known or conventional device capable of practicing the desired function, such as a spring material forming the elongated restriction member into the loop, so that the restriction opening has a predetermined size, and the adjustment device may adjust the restriction member against the spring action of the spring material. In other words, the restriction member may comprise a spring clip. The spring material may be integrated in the restriction member.

Preferably, the adjustment device comprises a movement transferring member, suitably a drive wheel, in engagement with at least one of the end portions of the restriction member and operable to displace the one end portion relative to the other end portion of the restriction member. The drive wheel may advantageously be in engagement with both of the end portions of the restriction member and be operable to displace said end portions relative to each other. An elongated flexible drive shaft may be operatively connected to the drive wheel, for transferring manual or motor generated power from a location remote from the restriction member. In its simplest embodiment, the drive wheel may comprise a pulley in frictional engagement with the restriction member. As an alternative, a gear rack may be formed on at least one of the end portions of the restriction member and the drive wheel may comprise a gear wheel in mesh with the gear rack. Other suitable known or conventional mechanisms may also or alternatively be used as the adjustment device.

The movement transferring member may alternatively comprise at least one cylinder and a piston, which is movable therein and is connected to one of the end portions of the restriction member, the piston being operable to longitudinally displace the one end portion of the restriction member relative to the other end portion of the restriction member. Alternatively, the movement transferring means may comprise two interconnected cylinders and two pistons in the respective cylinders connected to said end portions, respectively, of the restriction member, the pistons being operable to longitudinally displace the end portions of the restriction member relative to each other. Other known or conventional devices also or alternatively can be used as the movement transferring member.

A motor, which is fixed relative to the main portion of the restriction member and has a rotating drive shaft operatively connected to the movement transferring member, may be positioned relative to the elongated restriction member such that the drive shaft extends transverse thereto. Alternatively, the motor may be positioned relative to the elongated restriction member such that the drive shaft extends substantially tangentially to the loop of the restriction member.

In another embodiment utilizing the first adjustment principle, the elongated restriction member is longitudinally resilient and the adjustment device comprises a contraction device for longitudinally contracting the resilient restriction member. Preferably, the elongated restriction member comprises a substantially non-resilient main portion and an end portion forming an elongated helical spring, which is contractible by the contraction device. The contraction device may suitably comprise an elongated flexible pulling member connected to the main portion of the restriction member and extending through the helical spring to contract the helical spring against an arresting member, which is fixed relative to the main portion of the restriction member. The pulling member may extend in an elongated tube joined at one end thereof to the arresting member, so that a motor remote from the restriction member may be attached to the other end of the elongated tube and pulls the pulling member through the tube to contract the helical spring.

In yet another embodiment utilizing the first adjustment principle, the elongated restriction member comprises an elongated helical spring having a free end, and a body to which the spring is nonrotatably secured at its opposite end. The adjustment device rotates the helical spring in one direction to enlarge the coils of the helical spring to longitudinally contract the spring and to rotate the spring in the opposite direction to reduce the size of the coils of the spring to longitudinally extend the spring. As a preferred alternative, the restriction member comprises a further elongated helical spring having a free end and nonrotatably secured to the body at its opposite end, and the adjustment device comprises a drive shaft having two opposite end portions connected to the springs, respectively, at their free ends, the helical coils forming left and right hand helices, respectively. The adjustment device may alternatively comprise a gearing having an input shaft and two opposite aligned output shafts connected to the helical springs, respectively, at their free ends, the input shaft being connected to said output shafts so that the output shafts rotate in the opposite directions upon rotation of the input shaft, the helical coils forming the same helices. In accordance with a second adjustment principle, the adjustment device mechanically adjusts the restriction member so that at least a portion of a radially innermost circumferential confinement surface formed by the restriction member is substantially radially displaced. In one embodiment utilizing the second adjustment principle, the restriction member comprises an elongated voltage responsive element forming part of the confinement surface and capable of bending into a bow in response to a voltage applied across the element, the radius of curvature of the bow being adjustable by changing the level of the voltage. In another embodiment utilizing the second adjustment principle, the adjustment device changes the diameter of an elastic annular element of the restriction member, which forms the confinement surface. Preferably, the forming means comprises a substantially rigid outer annular element coaxially surrounding the elastic annular element, and the adjustment device comprises means for pulling the elastic annular element radially outwardly towards the outer annular element to expand the elastic annular element. For example, the pulling means may comprise a plurality of threads secured to the elastic annular element along the circumference thereof and running from the elastic annular element via guide members attached to the outer annular element.

In yet another embodiment utilizing the second adjustment principle, the forming means comprises a substantially rigid outer annular element, and the restriction member comprises an elongated helical spring extending internally along the outer annular element and contacting the latter. The helical spring forms part of the circumferential confinement surface and has a free end. The restriction member further comprises a body to which the spring is nonrotatably secured at its opposite end. The adjustment device rotates the helical spring in one direction to enlarge the coils of the spring to contract the circumferential confinement surface and rotates the spring in the opposite direction to reduce the size of the coils of the spring to expand the circumferential confinement surface. As an alternative, which is preferred, the restriction member comprises two elongated helical springs forming part of the circumferential confinement surface and connected to the body of the restriction member. The adjustment device rotates each spring in one direction to enlarge the coils of the spring to contract the circumferential confinement surface and rotates the spring in the opposite direction to reduce the size of the coils of the spring to expand the circumferential confinement surface.

In accordance with a third adjustment principle, the restriction member comprises at least two separate elements, at least one of which is pivoted so that it may turn in a plane in which the restriction member extends, and the adjustment device turns the pivoted element to change the size of the restriction opening. Preferably, the restriction member comprises a plurality of separate pivoted elements disposed in series, each pivoted element being turnable in the plane, and the adjustment device turns all of the pivoted elements to change the size of the restriction opening. For example, the pivoted elements may comprise lamellae arranged like the conventional adjustable aperture mechanism of a camera. In accordance with a fourth adjustment principle, the adjustment device folds at least two foldable frame elements of the restriction member towards each other. Preferably, the foldable frame elements comprise two substantially or partly semi-circular frame elements which are hinged together so that the semi-circular elements are swingable relative to each other from a fully open state in which they form part of a circle to a fully folded state in which they form part of a semi-circle. The same principal may be used with the swingable parts mounted together in one end and not in the other end. Alternatively, the restriction device may comprises two preferable rigid articulated clamping elements positioned on opposite sides of a vein like the blades of a scissor, and the adjustment device turns the clamping elements toward each other to clamp the vein, corpus cavernosum, crura or the prolongation thereof between the clamping elements, thereby restricting the blood flow in the vein, corpus cavernosum, crura or the prolongation thereof.

In accordance with a fifth adjustment principle, the adjustment device turns the restriction member around a longitudinal extension thereof, the elongated restriction member being elastic and varying in thickness as seen in a cross-section therethrough. Suitably, the elongated restriction member comprises an elastic belt.

In accordance with a sixth adjustment principle, the adjustment device changes the size of the restriction opening such that the outer circumferential confinement surface of the restriction member is changed.

In accordance with a seventh adjustment principle, the adjustment device changes the size of the restriction opening such that the outer circumferential confinement surface of the restriction member is unchanged. In accordance with an eighth adjustment principle, the elongated restriction member may be flexible, and the adjustment device pulls a first portion of the flexible restriction member from a second portion of the flexible restriction member opposite the first portion in the loop to squeeze a vein, the corpus cavernosum, the crura or the prolongation thereof between two opposite lengths of the elongated flexible restriction member to restrict the blood flow in the vein, corpus cavernosum, crura or the prolongation thereof. In accordance with a ninth adjustment principle, the restriction device comprises two rigid elements on opposite or different sides of a vein, the corpus cavernosum, the crura or the prolongation thereof, and the adjustment device decreases the distance between the rigid elements to squeeze the vein, corpus cavernosum, crura or the prolongation thereof between the rigid elements, thereby restricting the blood flow in the vein, corpus cavernosum, crura or the prolongation thereof.

In accordance with a tenth adjustment principle, the restriction device bends or rotates a portion of a vein, the corpus cavernosum, the crura or the prolongation thereof to restrict the blood flow in the same. For example, the restriction device may comprise at least two bending members, such as cylindrical or hour-glass shaped rollers, positioned on opposite or different sides of the vein and displaced relative to each other along the vein, corpus cavernosum, crura or the prolongation thereof, and the adjustment device may move the bending members against the vein, corpus cavernosum, crura or the prolongation thereof to bend the latter to restrict the blood flow in the vein, corpus cavernosum, crura or the prolongation thereof. Suitably, the displacement members may comprise rollers. The restriction device may also rotate a portion of the esophagus or stomach. The bending or rotating members may take any shape. With the prolongation of the corpus cavernosum or crura should be understood the penile tissue extended inside the human body and following the pathway of the blood flow leaving the penis. Alternatively, the two bending members one placed more distal than the other may be rotated in opposite direction relative to each other. With interconnecting material for example flexible bands between. the holding members a restriction will occur between the bending members when they are rotated.

The restriction device may in all applicable embodiments take any shape and be either hydraulic or non-inflatable. In all of the above-described embodiments the adjustment device is conveniently operated by any suitable motor, preferably an electric motor, which may be fixed directly to or be placed in association with the restriction device, or alternatively be located remote from the restriction device, advantageously in the abdomen or subcutaneously. In the latter alternative the motor is advantageously connected to the adjustment device by a flexible power transmission conduit to permit a suitable positioning of the motor in the abdomen of the patient. The motor may be manually activatable, for example by an implanted switch.

In some of the above described embodiments of the invention, however, the adjustment device may conveniently be operable by a hydraulic operation device, which preferably is manually activatable. The hydraulic operation device may advantageously include hydraulic servo means to facilitate manual activation. As an alternative, the hydraulic device may be powered by an electric motor, which may be manually activatable or controlled by remote control means. The components of such a hydraulic operation device may be placed in association with the restriction device and/or be located at a suitable place in the abdomen or subcutaneously.

More specifically, a reservoir may be provided containing a predetermined amount of fluid for supplying the hydraulic operation device with fluid. The reservoir defines a chamber for the predetermined amount of fluid and the hydraulic operation device changes the size of the chamber. The hydraulic operation device may comprise first and second wall portions of the reservoir, which are displaceable relative to each other to change the size of the chamber of the reservoir. The first and second wall portions of the reservoir may be designed to be displaceable relative to each other by manual manipulation thereof, preferably to permit manual pushing, pulling or rotation of any of the wall portions in one direction. Alternatively, the wall portions may be displaceable relative to each other by magnetic means (such as a permanent magnet and magnetic material reed switch, or other known or conventional magnetic devices), hydraulic means or electrical control means such as an electric motor. The magnetic means, hydraulic means, or electrical control means may all be activated by manual manipulation, preferably using a subcutaneously located manually manipulatable device. This control may be indirect, for example via a switch.

The hydraulic operation device may operate the adjustment device with fluid from the reservoir in response to a predetermined first displacement of the first wall portion of the reservoir relative to the second wall portion of the reservoir, to adjust the restriction device to release the tissue, and to operate the adjustment device with fluid from the reservoir in response to a predetermined second displacement of the first wall portion of the reservoir relative to the second wall portion of the reservoir, to adjust the restriction device to restrict the blood flow leaving the penis. In this embodiment, no pump is used, only the volume of the reservoir is varied. This is of great advantage compared to the solution described below when a pump is used to pump fluid between the reservoir and the adjustment device because there is no need for a non-return valve and it is still possible to have fluid going both to and from the reservoir. As an alternative, the hydraulic operation device may comprise a pump for pumping fluid between the reservoir and the adjustment device. The pump may pump fluid both to and away from the adjustment device, or hydraulic means controlling the adjustment device. A mechanical manual solution is proposed in which it is possible to pump in both directions just by pushing an activating member in one direction. Another alternative is a pump pumping in only one direction and an adjustable valve to change the direction of fluid to either increase or decrease the amount of fluid in the reservoir. This valve may be manipulated manually, mechanically, electrically, magnetically, or hydraulically. Any kind of motor could of course be used for all the different operations as well as wireless remote solutions. The pump may comprise a first activation member for activating the pump to pump fluid from the reservoir to the adjustment device and a second activation member for activating the pump to pump fluid from the adjustment device to the reservoir. The activation members may be operable by manual manipulation, preferably to permit manual pushing, pulling or rotating thereof in one direction. Suitably, at least one of the activation members is adapted to operate when subjected to an external pressure exceeding a predetermined magnitude.

Alternatively, at least one of the first and second activating members may be operable by magnetic means, hydraulic means or electrical control means such as an electric motor. The magnetic means, hydraulic means, or electrical control means may all be activated by manual manipulating means preferably located subcutaneously. This activation may be indirect, for example via a switch.

Advantageously, especially when manual manipulation means are used, a servo means could be used. With servo means less force is needed for operating the adjustment device. The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible.

Alternatively, a reverse servo may be employed. The term “reverse servo” is to be understood as a mechanism that transfers a strong force acting on a moving element having a short stroke into a weak force acting on another moving element having a long stroke; i.e. the opposite function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system will affect the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device. The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the servo and reverse servo may be used in connection with all of the various components and solutions described in the present specification.

Preferably, the reverse servo comprises hydraulic means and a main fluid supply reservoir and eventually an additional fluid supply reservoir. Both reservoirs define a chamber containing hydraulic fluid, and the hydraulic means comprises first and second wall portions of the main fluid supply reservoir, which are displaceable relative to each other to change the volume of the chamber of the main fluid supply reservoir. The hydraulic means may control the adjustment device indirectly, e.g. via an increased amount of fluid in the main fluid supply reservoir, in response to a predetermined first displacement of the first wall portion of any of the reservoirs relative to the second wall portion of the reservoir to restrict the blood flow leaving the penis, and to control the adjustment device in response to a second displacement of the first wall portion of any reservoir relative to the second wall portion, to indirectly adjust the restriction device to release the penile tissue. The wall portions of the reservoirs may be designed to be displaceable relative to each other by manual manipulation thereof or be displaceable relative to each other by manually pushing, pulling or rotating any of the wall portions of the reservoir in one direction. Alternatively, the wall portions of the main fluid supply reservoir may be displaceable relative to each other by magnetic means, hydraulic means or electric control means including an electric motor.

The magnetic means, hydraulic means, or electrical control means may all be activated by manually manipulated means preferably located subcutaneously. This control may be indirect for example via a switch.

Even in the broadest embodiment the adjustment device may comprise a servo means. The servo means may comprise a hydraulic operation means, an electrical control means, a magnetic means, mechanical means or a manual manipulation means. The hydraulic operation means, electrical control means, mechanical means or magnetic means may be activated by manual manipulating means. Using a servo system will save the use of force when adjusting the adjustment device which may be of importance in many applications, for example when a battery cannot put out enough current although the total energy in the battery is more than enough to power the system.

In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable. All solutions may be controlled by a wireless remote control for controlling the adjustment device. The remote control may advantageously be capable of obtaining information related to the blood flow leaving the penis or the blood pressure or other important physical parameters and of commanding the adjustment device to adjust the restriction device in response to obtained information. With the wireless remote control the apparatus is conveniently controlled by the patient when he so desires, which is of great advantage compared to the prior art procedures. With the remote control the apparatus is conveniently controlled to adjust the implanted restriction device, which controls the blood flow leaving the penis. The restriction device may be operable to open and close the blood flow passageway formed by the penile exit veins. The restriction device may steplessly control the cross-sectional area of the passageway. The apparatus may further comprise a pressure sensor for directly or indirectly sensing the pressure against the restriction device and the restriction device may control the blood flow in response to signals from the pressure sensor. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor. The adjustment device preferably non-invasively adjusts the restriction device to change the size of the cross-sectional area. The adjustment device and/or other energy consuming components of the apparatus may be energized with wirelessly transmitted energy from outside the patient's body or be powered by an implanted battery or accumulator.

The apparatus may further comprise an implanted energy transforming device for transforming wireless energy directly or indirectly into kinetic energy for operation of the restriction device. The energy transforming device may, preferably directly, transform he wireless energy in the form of sound waves into electric energy for operation of the restriction device. Suitably the energy transforming device comprises a capacitor adapted to produce electric pulses from the transformed electric energy. The apparatus may further comprise an energy transfer means for wireless transfer of energy from outside the patient's body to the adjustment device and/or other energy consuming implantable components of the apparatus. The energy transfer means may be adapted to intermittently transfer the energy, preferably electric energy, in the form of a train of energy pulses for direct use in connection with the energizing of the energy consuming components of the apparatus. An implanted capacitor having a capacity less than 0.1 μF may be used for producing the train of energy pulses.

A motor may be implanted for operating the adjustment device, wherein the energy transfer means is adapted to directly power the motor with transferred energy. Alternatively, or in combination with the motor, a pump may be implanted for operating the adjustment device, wherein the energy transfer means is adapted to transfer wireless energy in the form of electromagnetic waves for direct power of the pump. Preferably, the pump is not a plunger type of pump, but may comprise a peristaltic or membrane pump.

The energy transfer means preferably transfers wireless energy in the form of electromagnetic waves. However, for safety radio waves may be excluded.

Alternatively, the energy transferred by the energy transfer means may comprise an electric field or a magnetic field.

Most preferred, the energy transferred by the energy transfer means comprises a signal.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter.

The apparatus may further comprise an implanted energizer unit for providing energy to energy consuming implanted components of the apparatus, such as electronic circuits and/or a motor for operating the adjustment device. The apparatus may comprise an external energy transmitter for transmitting wireless energy, wherein the energizer unit is adapted to transform the wireless energy into electric energy. An implanted electric motor may operate the adjustment device and the energizer unit may be adapted to power the electric motor with the electric energy transformed from the wireless energy. The energizer unit may comprise a battery and a switch operable by the wireless energy transmitted by the external transmitter, for connecting the battery to the implanted energy consuming components of the apparatus in an “on” mode when the switch is powered by the wireless energy and to keep the battery disconnected from the energy consuming components in a “standby” mode when the switch is not powered.

The control unit may power such an implanted motor with energy provided by the energizer unit in response to a control signal received from the signal transmitter. Any known or conventional signal transmitter or signal receiver that is suitable for use with a human or mammal patient may be provided as the signal transmitter or signal receiver of the invention.

Generally, all the signals mentioned above may comprise electromagnetic waves, such as infrared light, visible light, laser light, micro waves, or sound waves, such as ultrasonic waves or infrasonic waves, or any other type of wave signals. The signals may also comprise electric or magnetic fields, or pulses. All of the above-mentioned signals may comprise digital signals. The control signals may be carried by a carrier wave signal, which in an alternative embodiment may be the same signal as the wireless energy signal. Preferably a digital control signal may be carried by an electromagnetic wave signal. The carrier wave or control signal may be amplitude or frequency modulated.

The motor may be any type of motor, such as a pneumatic, hydraulic or electric motor and the energizer unit may power the motor with pressurized gas or liquid, or electric energy, depending on the type of motor. Where the motor is an electric
motor, it may power pneumatic or hydraulic equipment.

The energizer unit may comprise a power supply and the control unit may power the motor with energy from the power supply. Preferably, the power supply is an electric power supply, such as a battery, and the motor is an electric motor. In this case, the battery also continuously powers at least part of the circuitry of the signal receiver in a standby mode between the adjustments, in order to keep the signal receiver prepared for receiving signals transmitted from the signal transmitter.

The energizer unit may transfer energy from the control signal, as the control signal is transmitted to the signal receiver, into electric energy for powering the implanted electronic components. For example, the energizer unit may transfer the energy from the control signal into a direct or alternating current.

In case there is an implanted electric motor for operating the adjustment device the energizer unit may also power the motor with the transferred energy. Advantageously, the control unit directly powers the electric motor with electric energy, as the energizer unit transfers the signal energy into the electric energy. This embodiment is particularly simple and does not require any recurrent invasive measures for exchanging empty power supplies, such as batteries, that is required in the first embodiment described above. The motor may also be directly powered with wirelessly transmitted electromagnetic or magnetic energy in the form of signals, as the energy is transmitted. All the various functions of the motor and associated components described in the present specification may be used where applicable.

For adjustment devices of the type that requires more, but still relatively low, power for its operation, the energizer unit may comprise a rechargeable electric power supply for storing the electric energy obtained and the control unit may power the electric motor with energy from the rechargeable electric power supply in response to a control signal received from the signal transmitter. In this case, the rechargeable power supply can be charged over a relatively long time (e.g. a few seconds up to a half hour) without powering the electric motor.

The electric power supply suitably comprises an inexpensive simple capacitor. In this case, the electric motor may be a stepping motor. In all embodiments the motor may. preferable be able to perform a reversing function. The signal transmitter may transmit an electromagnetic. signal and the energizer unit may draw radiant energy from the electromagnetic wave signal, as the latter is transmitted to the signal receiver, and transfer the radiant energy into electric energy.

Alternatively, the energizer unit may comprise a battery or accumulator, an electrically operable switch adapted to connect the battery to the signal receiver in an on mode when the switch is powered and to keep the battery disconnected from the signal receiver in a standby mode when the switch is not powered, and a rechargeable electric power supply for powering the switch. The control unit may power the electric motor with energy from the battery in response to a control signal received from the signal transmitter, when the switch is in its on mode. Advantageously, the energizer unit may transform wave energy from the control signal, as the latter is transmitted to the signal receiver, into a current for charging the rechargeable electric power supply, which suitably is a capacitor. Energy from the power supply is then used to change the switch from off (standby mode) to on. This embodiment is suited for adjustment devices of the type that require relatively high power for their operation and has the advantage that the electronic circuitry of the signal receiver does not have to be powered by the battery between adjustments. As a result, the lifetime of the battery can be significantly prolonged. The switch may be switched manually or by the use of magnetic or electric energy. As an example, the signal transmitter may transmit an electromagnetic wave signal and the energizer unit may draw radiant energy from the electromagnetic wave signal, as the latter is transmitted to the signal, receiver, and may transfer the radiant energy into said current. The energizer unit suitably comprises a coil of the signal receiver for inducing an alternating current as the electromagnetic wave signal is transmitted through the coil and a rectifier for rectifying the alternating current. The rectified current is used for charging the rechargeable power source.

Alternatively, the signal transmitter and receiver may solely be used for a control signal and a further pair of signal transmitter and receiver may be provided for transferring signal energy to implanted components. By such a double system of signal transmitters and receivers the advantage is obtained that the two systems can be designed optimally for their respective purposes, namely to transmit a control signal and to transfer energy from an energy signal. Accordingly, the apparatus may further comprise an external energy transmitter for transmitting wireless energy, wherein the energizer unit comprises a battery and an operable switch for connecting the battery to the signal receiver in an “on” mode when the switch is powered and for keeping the battery disconnected from the signal receiver in a “standby” mode when the switch is not powered, and the external energy transmitter powers the switch. Suitably, the energy transmitter may directly power the switch with the wireless energy to switch into the “on” mode. As should be realized by a skilled person, in many of the above-described embodiments the adjustment device may be operated by control means or manual manipulation means implanted under the skin of the patient, such as a pump, an electrical switch or a mechanical movement transferring means. In the manual embodiment it is not necessary to use a motor for operating the adjustment device.

In embodiments including hydraulic transmission means, an injection port connected to the hydraulic means may be provided for enabling, normally single, once-and-for-all, calibration of the amount of fluid in the hydraulic system.

In all embodiments a motor may be operatively connected to the adjustment device. A reversing device may be implanted in the patient for reversing the motor. The adjustment device preferably adjusts the restriction device in a non-manual manner without the patient touching his skin. The adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces.

All the above-described various components, such as the motor, pump and capacitor, may be combined in the different embodiments where applicable. Also the various functions described in connection with the above embodiments may be used in different applications, where applicable.

All the various ways of transferring energy and controlling the energy presented in the present specification may be practiced by using all of the various components and solutions described.

The invention also provides a method for treating male sexual impotence, comprising surgically implanting in the body of a male patient suffering from sexual impotence an adjustable restriction device which directly engages a portion of the normal penile tissue of the patient to affect the blood flow leaving the penis and the prolongation thereof, and when desired to achieve erection, mechanically adjusting the restriction device to temporarily restrict the blood flow leaving the penis. The method may further comprise implanting the male sexual impotence treatment apparatus in the base of the patient's penis or its prolongation, preferably implanting an restriction device engaging the corpora cavernosa, crura or the prolongation thereof as a single unit or engaging the two corpora cavernosa or crura or the prolongations thereof separately. The method may comprise implanting the restriction device engaging one or both of the crura of the penis.

As a modification, the method may further comprise implanting the restriction device engaging at least one of the exit veins from the penis.

The invention provides another method for treating male sexual impotence, comprising the steps of: placing at least two laparascopical trocars in the body of a male patient suffering from sexual impotence, inserting a dissecting tool through the trocars and dissecting an area of the penis and abdominal or peritoneal surroundings, placing at least one adjustable restriction device in the dissected area engaging the penile tissue or the prolongation thereof, and adjusting the restriction device to restrict the blood flow leaving the penis when the patient wishes to achieve erection.

The method may further comprise mechanically adjusting said restriction device in a non-manual manner. The restriction device may engage (a) both of the corpora cavernosa, the crura of the penis or the prolongation thereof as a single unit; or (b) one of the exit veins from the penis.

Alternatively, the method may further comprise implanting (a) a further adjustable restriction device, wherein the two restriction devices engage the two corpora cavernosa, the crura of the penis or their prolongations, respectively, as separate units; or (b) several restriction devices engaging respective exit veins from the penis. The method may further comprise implanting a source of energy in the patient and providing a control device for controlling the source of energy from outside the patient's body to supply energy to the restriction device.

### AC: Impotence Treatment

The present aspect relates to a male sexual impotence treatment apparatus, comprising an operable restriction device implantable in a patient for engaging the penile tissue or the prolongation thereof to form a restricted blood, flow passageway through which blood flow leaves the penis. The restriction device is operable to restrict the passageway to provide erection. The expression “penile tissue or the prolongation thereof” should be understood to mean the penile tissue extended inside the human body and following the pathway of the blood flow leaving the penis i.e. one or more exit veins from the penis, the corpus cavernosum, crura or the prolongation thereof.

The male sexual impotence apparatus is characterized in that a source of energy is provided, and a control device operable from outside the patient's body is provided for controlling the source of energy to release energy for use in connection with the operation of the restriction device, when the restriction device is implanted.

As a result, the advantage is achieved that the restriction device can be operated without need for touching subcutaneously implanted components of the apparatus, when the restriction device shall be adjusted. Furthermore, the apparatus provides simple and effective control of the energy supplied to implanted components of the apparatus which ensures an extended and reliable functionality of the apparatus, possibly for the rest of the patient's natural life, and at least many years.

The restriction device may be implanted in the base of the patient's penis or the prolongation thereof and preferably may engage the corpus cavernosum, crura or the prolongation thereof of the penis. However, there are several alternative positions of the restriction device that give more or less satisfactory restriction of the blood flow leaving the penis. Thus, as a first alternative the restriction member may extend around both corpora cavernosa or crura of the penis as\hat{ } a single unit. As a second alternative the restriction device may comprise two elongated restriction members extending around the respective corpora cavernosa or crura. As a third alternative the elongated restriction member may encircle one or more of the penile exit veins. As a fourth alternative the restriction device may comprise several restriction members extending around the respective penile exit veins.

The restriction device preferably controls the size of the cross-sectional area of the passageway through which blood flow leaves the penis, which gives the advantage that the patient is enabled to adjust the restriction device to achieve the desired erection without feeling pain. This advantage should not be underestimated, because fine adjustments to decrease the cross-sectional area of the passageway, will allow maximal erection with a minimum of restriction. The control device may also control the restriction device. The control device may comprise an internal control unit, preferably. including a microprocessor, implanted in the patient for controlling the restriction device. The control device may further comprise an external control unit outside the patient's body, wherein the internal control unit is programmable by the external control unit, for example for controlling the restriction device over a short period of time. Alternatively, the internal control unit may control the restriction device over time in accordance with an activity schedule program, which may be adapted to the patient's needs. For example to avoid an abnormally high pressure against the human tissue.

Conveniently, the external control unit may load the internal control unit with data in accordance with a loading mode only authorized for a doctor. For specialized controls of the restriction device, the external control unit may control the internal control unit in accordance with a doctor mode only authorized for the doctor. For simple controls of the restriction device, the external control unit may control the internal control unit in accordance with a patient mode permitted for the patient. Thus, by using the external control unit in accordance with different modes it is possible to have certain functions of the restriction device controlled by the patient and other more advanced functions controlled by the doctor, which enables a flexible post-operation treatment of the patient.

The control device may be adapted to control the source of energy to release energy, for instance to intermittently release energy in the form of a train of energy pulses, for direct use in connection with the operation of the restriction device. In accordance with a suitable embodiment the control device controls the source of energy to release electric energy, and the apparatus further comprises an implantable capacitor for producing the train of energy pulses from the released energy. In this case the term “direct” is used to mean, on one hand, that the released energy is used while it is being released by the control device, on the other hand, that the released energy may be somewhat delayed, in the order of seconds, by for instance an energy stabilizer before being used in connection with the operation of the restriction device. The restriction device may be operable in non-manual, a non-magnetic or non-mechanical manner by use of the released energy. In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

Generally, the apparatus further comprises an operation device implantable in the patient for operating the restriction device, wherein the control device controls the operation device to operate the restriction device. The control device may directly power the operation device with energy released from the source of energy and/or power other implantable energy consuming components of the apparatus. In this case the term “directly” is used to mean, on one hand, that the operation device is powered with released energy while the latter is being released by the control device, on the other hand, that the released energy may be somewhat delayed, in the order of seconds, by for instance an energy stabilizer before powering the operation device. The advantage of directly using energy as it is released is that the apparatus can be of a very simple design and the few components involved makes the apparatus reliable. The restriction device may be non-inflatable, i.e. with no hydraulic fluid involved for the adjustments of the restriction device. This eliminates problems with fluid leaking from the restriction device. The operation device may comprise hydraulic means and at least one valve for controlling a fluid flow in the hydraulic means. The control device may suitably comprise a wireless remote control for controlling the valve. The restriction device may comprise hydraulic means and the operation device may comprise a reservoir forming a fluid chamber with a variable volume connected to the hydraulic means. The operation device may distribute fluid from, the chamber to the hydraulic means by reduction of the volume of the chamber and withdraw fluid from the—hydraulic means to the chamber by expansion of the volume of the chamber.

In accordance with a first main aspect of the invention, the source of energy is external to the patient's body and the control device controls the source of energy to release wireless energy. The external source of energy may be of any conceivable kind, such as a nuclear source of energy or a chemical source of energy.

An energy storage device, preferably an electric accumulator, may be implanted in the patient for storing the wireless energy released from the external source of energy. The electric accumulator may comprise at least one capacitor or at least one rechargeable battery, or a combination of at least one capacitor and at least one rechargeable battery. Alternatively, a battery may be implanted in the patient for supplying electric energy to implanted electric energy consuming components of the apparatus, in addition to the supply of wireless energy. Where the control device comprises an implantable control unit the electronic circuit thereof and the restriction device may be directly powered with transformed wireless energy, or energy from either the implanted energy storage device or battery.

In accordance with a second main aspect of the invention, the wireless energy is directly used for operation of the restriction device, i.e. the restriction device is operated as the wireless energy is released from the external source of energy by the control device. In this case the term “directly” is used to mean, on one hand, that the restriction device is promptly operated by using the released energy without first storing the latter, on the other hand, that the released energy may. be somewhat delayed, in the order of seconds, by for instance an energy stabilizer before being used for the operation of the restriction device. As a result, a very simple control of the restriction device is achieved and there are only a few implanted components of the apparatus. For example, there is no implanted source of energy, such as a battery, nor any implanted complicated signal control-system. This gives the advantage that the apparatus will be extremely reliable.

Generally, the control device controls and directly or indirectly powers the operation device with wireless energy released from the source of energy and/or powers other implanted energy consuming components of the apparatus. In a first particular embodiment in accordance with the first and second main aspects of the invention, the operation device comprises a motor, preferably an electric motor which may have electrically conductive parts made of plastics. The motor may include a rotary motor, wherein the control device is adapted to control the rotary motor to rotate a desired number of revolutions. Alternatively, the motor may include a linear motor, or a hydraulic or pneumatic fluid motor, wherein the control device is adapted to control the fluid flow through the fluid motor. Motors currently available on the market are getting smaller and smaller. Furthermore, there is a great variety of control methods and miniaturized control equipment available. For example, a number of revolutions of a rotary motor may be analyzed by a Hall-element just a few mm in size.

In a second particular embodiment\hat{ } in accordance with the first and second main aspects of the invention, the control device is adapted to shift polarity of the released energy to reverse the operation device. The operation device may suitably comprise an electric motor and the released energy may comprise electric energy.

In a third particular embodiment in accordance with the first and second main aspects of the invention, the restriction device is operable to perform a reversible function and there is a reversing device implantable in the patient for reversing the function performed by the restriction device. Such a reversing function preferably involves enlarging and restricting the blood flow passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch. In accordance with the third particular embodiment, the operation device preferably comprises a motor, wherein the reversing device reverses the motor.

In a fourth particular embodiment in accordance with the first and second main aspects of the. invention, the restriction device comprises hydraulic means, for example including an expansible/contractible cavity for fluid. Preferably, the operation device is adapted to conduct hydraulic fluid in the hydraulic means, and comprises a motor, a valveless fluid conduit connected to the hydraulic means of the restriction device, and a reservoir for fluid, wherein the reservoir forms part of the conduit. The operation device suitably comprises a pump operated by the motor. All of the hydraulic components involved are preferably devoid of any non-return valve. This is of great advantage, because with valves involved there is always a risk of malfunction due to improperly working valves, especially when long time periods pass between valve operations. The reservoir may form a fluid chamber with a variable volume, and the pump may distribute fluid from the chamber to the hydraulic means of the restriction device by reduction of the volume of the chamber and withdraw fluid from the hydraulic means to the chamber by expansion of the volume of the chamber. In accordance with a third main aspect of the invention, the source of energy is implantable in the patient. Thus, when the source of energy is implanted in a patient the control device controls it from outside the patient's body to release energy. This solution is advantageous for embodiments of the apparatus that have a relatively high consumption of energy which cannot be satisfied by direct supply of wireless energy.

The implantable source of energy may comprise an accumulator, preferably an electric source of energy, such as a battery having a lifetime of at least 10 years. In accordance with a fourth main aspect of the invention, the apparatus comprises a switch implanted in the patient for directly or indirectly switching the operation of the restriction device and an internal source of energy, such as a battery, implanted in the patient for supplying energy for the operation of the restriction device, wherein the switch directly or indirectly affects the supply of energy from the internal source of energy. This solution is advantageous for embodiments of the apparatus that have a relatively high energy consumption which cannot be met by direct supply of wireless energy.

In a first particular embodiment in accordance with the fourth main aspect of the invention, the switch switches between an off mode, in which the internal source of energy is not in use, and an on mode, in which the internal source of energy supplies energy for the operation of the restriction device.

In this case, the switch is conveniently operated by the wireless energy released from the external source of energy to switch between the on and off modes. The control device, preferably comprising a wireless remote control, may control the external source of energy to release the wireless energy. The advantage of this embodiment is that the lifetime of the implanted source of energy, such as a battery, can be significantly prolonged, since the implanted source of energy does not supply energy when the switch is in its off mode.

In a second particular embodiment in accordance with the fourth main aspect of the invention, the control device comprises a wireless remote control for controlling the internal source of energy. In this case, the switch is operable by the wireless energy from the external source of energy to switch between an off mode, in which the internal source of energy and remote control are not in use, and a standby mode, in which the remote control is permitted to control the internal source of energy to supply energy for the operation of the restriction device.

In a third particular embodiment in accordance with the fourth main aspect of the invention, the apparatus further comprises an energy transforming device implanted in the patient for transforming the wireless energy into storable energy, wherein the internal source of energy is capable of storing the storable energy. The internal source of energy preferably comprises an electric accumulator, at least one capacitor or at least one rechargeable battery, or a combination of at least one capacitor and at least one rechargeable battery. In this case, the switch switches from an off mode, in which the internal source of energy is not in use, to an on mode, in which the internal source of energy supplies energy for the operation of the restriction device.

The control device, preferably comprising a wireless remote control, may control the switch to switch between the on and off modes. Alternatively, in this third particular embodiment an energy storage device may be implanted in the patient for storing the storable energy instead of the internal source. of energy, wherein the switch is operable by energy from the implanted energy storage device to switch between an off mode, in which the internal source of energy is not in use, and an on mode, in which the internal source of energy supplies energy for the operation of the restriction device. In this case, the control device (the wireless remote control) controls the energy storage device to operate the switch. The internal source of energy preferably comprises an electric source of energy, such as an accumulator or a battery having a lifetime of at least 10 years. However, other kinds of sources are also conceivable, such as a nuclear source of energy or a chemical source of energy. The above first, second, third and fourth particular embodiments described in connection with the first and second main aspects are also applicable in accordance with the third main aspect of the invention, i.e. where the source of energy is to be implanted, and in accordance with the fourth main aspect of the invention, i.e. where the apparatus comprises an implantable switch.

All of the above embodiments may be combined with at least one implantable sensor for sensing at least one physical parameter of the patient, wherein the control device may control the restriction device in response to signals from the sensor. For example, the sensor may comprise—a pressure sensor for directly or indirectly sensing the pressure in the blood flow passageway. The expression n indirectly sensing the pressure in the blood flow passageway” should be understood to encompass the cases where the sensor senses the pressure against the restriction device or human tissue of the patient. The pressure sensor may be any suitable known or conventional pressure sensor such—as shown in U.S. Pat. Nos. 5,540,731, 4,846,181, 4,738,267, 4,571,749, 4,407,296 or 3,939,823; or an NPC-102 Medical Angioplasty Sensor. Where the control device comprises an internal control unit to be implanted in the patient, the internal control unit may suitably directly control the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to enlarge the blood flow passageway in response to the sensor sensing an abnormally high pressure against the restriction device. Where the control device comprises an external control unit outside the patient's body, the external control unit may, suitably directly, control the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor.

An external data communicator may be provided outside the patient's body and an internal data communicator to be implanted in the patient may be provided for communicating with the external data communicator. The internal data communicator may feed data related to the patient, or related to the restriction device, back to the external data communicator. Alternatively or in combination, the external data communicator may feed data to the internal data communicator. The internal data communicator may suitably feed data related to at least one physical signal of the patient.

Generally, the apparatus may comprise a switch to be implanted in the patient for directly or indirectly switching the energy released from the source of energy. For example, the restriction device may be operable to steplessly control the restriction of the blood flow passageway. A pressure sensor may be provided for directly or indirectly sensing the pressure in the blood flow passageway. The control device may control the restriction device in response to signals from the pressure sensor. The apparatus may comprise an implantable energy transforming device, wherein the control device releases electric energy and the energy transforming device transforms the electric energy into kinetic energy for, preferably direct, operation of the restriction device. Suitably, an implantable stabilizer, such as a capacitor or a rechargeable accumulator, or the like, may be provided for stabilizing the electric energy released by the control device. In addition, the control device may control the source of energy to release energy for a determined time period or in a determined number of energy pulses. Finally, the restriction device may be non-inflatable.

All of the above embodiments are preferably remote controlled. Thus, the control device advantageously comprises a wireless remote control transmitting at least one wireless control signal for controlling the restriction device. With such a remote control it will be possible to adapt the function of the apparatus to the patient's need. The wireless remote control may be capable of obtaining information on the condition of the restriction device and of controlling the restriction device in response to the information. Also, the remote control may be capable of sending information related to the restriction device from inside the patient's body to the outside thereof.

In a particular embodiment of the invention, the wireless remote control comprises at least one external signal transmitter or transceiver and at-least one internal signal receiver or transceiver implantable in the patient. In another particular embodiment of the invention, the wireless remote control comprises at least one external signal receiver or transceiver and at least one internal signal transmitter or transceiver implantable in the patient. The remote control may transmit a carrier signal for carrying the control signal, wherein the carrier signal is frequency, amplitude or frequency and amplitude modulated and is digital, analog or digital and analog. Also the control signal used with the carrier signal may be frequency, amplitude or frequency and amplitude modulated. The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray-radiation signal, or a gamma radiation signal. Where applicable, two or more of the above signals may be combined. The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

In all of the above solutions, the control device advantageously releases energy from the source of energy in a non-invasive, magnetic, non-magnetic, mechanical or non-mechanical manner.

The control device may release magnetic, electromagnetic, kinetic, sonic or thermal energy, or non-magnetic, non-sonic, non-thermal, non-electromagnetic or non-kinetic energy. The control device may be activated in a manual or non-manual manner to control the source of energy to release energy.

The operation device may be powered by magnetic energy, non-magnetic energy, electromagnetic energy, non-electromagnetic energy, kinetic energy, non-kinetic energy, thermal energy or non-thermal energy. However, preferably the operation device comprises an electrical operation device.

Typically the apparatus comprises an adjustment device for adjusting the restriction device to change the restriction of the blood flow passageway. The adjustment device may be adapted to mechanically adjust the restriction device. Alternatively, the adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces.

The above-presented embodiments of the invention may be modified in accordance with the following suggestions. The released energy may comprise electric energy and an implantable capacitor having a capacity less than 0.1 μF may be provided for producing the above-mentioned train of energy pulses.

An implantable motor or pump may be provided for operating the restriction device, wherein the control device is adapted to control the source of energy to directly power the motor or pump with the released energy. Specifically, the control device may be adapted to release wireless energy in the form of a magnetic field or electromagnetic waves (excluding radio waves) for direct power of the motor or pump, as the wireless energy is being released. Where a pump is used it preferably is not a plunger type of pump.

Generally, the wireless energy comprises a signal. The apparatus may further comprise implantable energy transforming device for transforming wireless energy directly or indirectly into energy different than the wireless energy, for operation of the restriction device. For example, the motor or pump may be powered by the transformed energy.

The energy transforming device may transform the wireless energy in the form of sound waves, preferably directly, into electric energy for operation of the restriction device. The energy transforming device may comprise a capacitor adapted to produce electric pulses from the transformed electric energy.

The motor mentioned in the present specification may also be directly powered with wirelessly transmitted electromagnetic or magnetic energy in the form of signals, as the energy is transmitted. Furthermore, all the various functions of the motor and associated components described in the present specification may be used where applicable. Generally, the restriction device advantageously is embedded in a soft or gel-like material, such as a silicone material having hardness less than 20 Shore.

Of course, the restriction device preferably is adjustable in a non-manual manner.

All the above described various components, such as the motor, pump and capacitor, may be combined in the different embodiments where applicable. Also the various functions described in connection with the above embodiments may be used in different applications, where applicable.

All the various ways of transferring energy and controlling the energy presented in the present specification may be practiced by using all of the various components and solutions described.

The present invention also provides a method of treating an impotent male patient, comprising: (a) surgically implanting in the male patient an operable restriction device engaging the penile tissue or the prolongation thereof to form a restrictable passageway for blood flow leaving the penis, (b) Providing a source of energy, (c) Controlling the source of energy to release energy for use in connection with the operation of the restriction device.

Steps (b) and (c) may further comprise providing a source of energy external to the patient's body and controlling the external source of energy from outside the patient's body to release wireless energy for use in connection with the operation of the restriction device. The method may further comprise (d) implanting in the patient an operation device which can adjust the—restrictable passageway in response to supplied energy, and (f) using the released wireless energy to operate the implanted operation device to enlarge and restrict, respectively, the passageway.

In accordance with an alternative ‘method, there is provided a method of treating an impotent male patient,” comprising the steps of placing at least two laparascopical trocars in the patient's body, inserting a dissecting tool through the trocars and dissecting an area of the penile tissue or the prolongation thereof, placing an operable restriction device in the dissected area in engagement with the penile—tissue or the prolongation thereof to form a restrictable passageway for the blood flow leaving the penis, providing a source of energy outside or inside the male patient's body, controlling the source of energy from outside the patient's body to release energy, which may comprise wireless energy where the source of energy is external to the patient's body, and using the released energy in connection with the operation of the restriction device.

In accordance with another alternative method, there is provided a method of treating an impotent male patient, comprising the steps of placing at least two laparascopical trocars in the male patient's body, inserting a dissecting tool through the trocars and dissecting an area of the penile tissue or the prolongation thereof, implanting an-operable restriction device in the dissected area in engagement with the penile tissue or the prolongation thereof to form a restrictable passageway for blood flow leaving the penis, implanting an energy transforming device, providing an external source of energy, controlling the external source of energy to release wireless energy, and transforming the wireless energy by the energy transforming device into energy different than the wireless energy for use in connection with the operation of the restriction device. This method may further comprise implanting a stabilizer in the patient's body to stabilize the energy transferred by the energy transforming device.

### AD: Urinary Incontinence Treatment with Wireless Energy Supply

The present aspect relates to a urinary incontinence treatment apparatus comprising an operable restriction device implantable in a patient, who suffers from urinary incontinence, for engaging the urethra or urine bladder, to form a restricted urine passageway in the urethra or urine bladder. The restriction device is operable to change the restriction of the urine passageway, i.e. to close and enlarge the urine passageway. The term “patient” includes an animal or a human being.

Urine incontinence is a widespread problem. Many people are helped through training of the muscles in the pelvic floor but too many have severe problems with urine leakage. Many different solutions to this problem have been tried. For example, there is a prior manually operated urine incontinence treatment apparatus having an artificial hydraulic sphincter device engaging the urethra and connected to an elastic reservoir implanted in the scrotum or in the region of the labia major. A disadvantage of this prior apparatus is that over time hard fibrosis is developed around the reservoir, which may cause malfunction of pumping components. Furthermore, it is a rather complicated task to manually squeeze the elastic implanted reservoir to pump hydraulic fluid to open the sphincter device when the patient needs to urinate. In particular women can get their fingers wet. The created fibrosis will sooner or later become a hard fibroid layer, which may make it even more difficult to pump the reservoir. Yet a further disadvantage is that the use of hydraulic fluid always entails a risk of fluid leaking from implanted hydraulic components.

A prior hydraulic apparatus designed to compress the urethra is disclosed in U.S. Pat. No. 5,520,606. A prosthetic sphincter with an inflatable cuff, which surrounds the urethra or encloses it on two sides, is disclosed in for example U.S. Pat. Nos. 4,571,749 and 4,222,377. U.S. Pat. No. 4,969,474 discloses a hydraulic method for treating both men and women with urinary incontinence problems in the same way. The apparatus of U.S. Pat. No. 4,969,474 includes a reservoir containing fluid and an inflatable compression means designed to compress urethra without risking tissue loss or necrosis to occur. An artificial hydraulically operated urethra sphincter employing an external magnet to achieve closure of the urethra cuff is disclosed in U.S. Pat. No. 5,562,598.

A prior mechanical prosthetic sphincter disclosed in U.S. Pat. No. 4,619,245 comprises a manually controllable actuating component for implanting at a convenient location in the patient's body.

The object of the present invention is to provide a new convenient urinary incontinence treatment apparatus, the performance of which may be affected by the patient at any time after operation, in particular when various needs arise over the course of a day, so that the patient substantially always is satisfied or comfortable.

This object is achieved by a urinary incontinence treatment apparatus of the kind stated initially characterized by an energy transmission device for wireless transmission of energy from outside the patient's body to inside the patient's body for use in connection with the operation of the restriction device including enlarging or restricting the urine passageway, when the restriction device is implanted.

As a result, the advantage is achieved that the urinary incontinence treatment apparatus provides simple and effective energy transmission that ensures long reliable function of the apparatus, possibly for the rest of the patient's life.

Generally, the apparatus comprises an energy transforming device implantable in the patient for transforming the energy wirelessly transmitted by the energy transmission device from a first form into a second form, preferably different than the first form.

The energy transforming device may comprise at least one semiconductor type of component or a circuitry of such semiconductor components. The semiconductor component may comprise a transistor or microchip or similar electronic components. However, the semiconductor component may not comprise rectifying diodes. In accordance with a main embodiment of the invention, the energy transforming device comprises at least one element having a positive region and a negative region and adapted to create an energy field between the positive and negative regions when exposed to the energy of the first form transmitted by the energy transmission device, so that the energy field provides the energy of the second form. Typically, the above-mentioned semiconductor component may include such an element.

In accordance with a preferred embodiment of the invention, the element comprises an electrical junction element capable of inducing an electric field between the positive and negative regions when exposed to the energy of the first form transmitted by the energy transmission device, whereby the energy of the second form comprises electric energy.

Consequently, the restriction device suitably is electrically operated, whereby the positive and negative regions of the electrical junction element supply electric energy for the operation of the restriction device. The apparatus suitably comprises implantable electric conductors connected to the positive and negative regions of the electrical junction element, whereby the electrical junction element is capable of supplying an electric current, such as a direct current, a pulsating direct current, a combination of a direct and pulsating direct current, an alternating current or a combination of a direct and alternating current, via the conductors. Furthermore, the electrical junction element may be capable of supplying a frequency, amplitude, or frequency and amplitude modulated analog, digital, or a combination of analog and digital signal, which is used in connection with control of the restriction device.

The element, preferably in the form of an electrical semiconductor junction element, should be designed to generate an output current exceeding 1 μA when exposed to the energy of the first form transmitted by the energy transmission device. Suitably the electrical junction element forms a flat and thin sheet and has a volume of less than 2000 cm3 to be suited for subcutaneous implantation, so that the electrical junction element can be located just behind the skin of the patient. Alternatively, it would be possible to implant the element in the thorax or cephalic region of the patient, or in an orifice of the patient's body and under the mucosa or intraluminar outside the mucosa of the orifice. Of course, all the components of the energy transforming device including the electrical junction element in contact with the patient's body should be of biocompatible material.

For in vitro appliances, a particular type of an electrical semiconductor junction element has been commonly used, namely a so-called p-n (positive/negative) junction element, typically in the form of solar cells. A solar cell transfers solar energy in the form of visible light into electric energy in the form of direct current. For example, a p-n junction element may comprise two layers of semiconductor, one p-type (positive) and the other n-type (negative), sandwiched together to form a “p-n junction”. This p-n junction induces an electric field across the element when absorbing quanta of light (photons).

To be more precise, the quanta of light transfer their energy to some of the semiconductor's electrons, which are then able to move about through the material. For each such negatively charged electron, a corresponding positive charge—a “hole”—is created. In an ordinary semiconductor, these electrons and holes recombine after a short time and their energy is wasted as heat. However, when the electrons and holes are swept across the p-n junction in opposite directions by the action of the electric field, the separation of charge induces a voltage across the p-n junction element. By connecting the p-n junction element to an external circuit, the electrons are able to flow thereby creating a current.

Surprisingly, it has been proved that although both the skin and subcutis absorb energy from an external light beam directed against the skin portion behind which a properly designed p-n junction element is located, the light energy transmitted through the skin can induce a current from the p-n junction element strong enough (minimum 1 μA) to enable the operation of the electrically operated restriction device. Thus, such a p-n junction element is now for the first time used for in vivo applications.

The apparatus may comprise an implantable pulse generator for generating electrical pulses from the energy of the second form produced by the energy field. Generally, the energy transforming device is adapted to transform the energy of the first form directly or indirectly into the energy of the second form. In accordance with a preferred embodiment of the invention, the energy of the second form comprises electric energy and the energy transforming device comprises a capacitor, which may be adapted to produce electric pulses from the transformed electric energy. Preferably, the capacitor may be adapted to produce the pulses as the energy transforming device transforms the energy of the first form transmitted by the energy transmission device into the electric energy of the second form. The capacitor should be small to facilitate implantation thereof; i.e. its capacity may not be more than 0.1 μF.

The apparatus may comprise an implantable stabilizer for stabilizing the energy of the second form. Where the energy of the second form comprises electric current the stabilizer may comprise at least one capacitor of the type described above. In most embodiments of the invention, the apparatus comprises implantable electrical components. Where the electrical components include a capacitor of the type described above or an accumulator, at least one, preferably a single, voltage level guard may advantageously be provided, wherein the charge and discharge of the capacitor or accumulator is controlled by use of the voltage level guard. As a result, there is no need for any implanted current detector and/or charge level detector for the control of the capacitor, which makes the apparatus simple and reliable. In a particular embodiment of the invention, the wireless energy of the first form comprises sound waves and the energy of the second form comprises electric energy, wherein the energy transforming device is adapted to directly transform the sound waves into electric energy. The apparatus may comprise an implantable motor or pump for operating the restriction device, wherein the motor or pump is powered by the transformed energy. In accordance with a main aspect of the invention, the energy transmission device may be adapted to transmit wireless energy for direct use in connection with the operation of the restriction device, as the wireless energy is being transmitted. The advantage of directly using energy as it is transmitted is that the apparatus can be of a very simple design and the few components involved makes the apparatus extremely reliable. For example, the energy transmission device may be adapted to directly power the motor or pump with wireless energy. The wireless energy may comprise a magnetic field or electromagnetic waves, suitably in the form of a signal, for direct power of the motor or pump. All the various functions of the motor and associated components described in the present specification may be used where applicable. As an alternative to the above-noted main aspect of the invention, the energy transforming device may be adapted to supply the energy of the second form for direct use in connection with the operation of the restriction device, as the energy of the first form is being transformed into the energy of the second form. Consequently, the energy transforming device may be adapted to directly power the motor or pump with the energy of the second form.

Generally, the energy transforming device directly operates the restriction device with the energy of the second form in a non-magnetic, non-thermal or non-mechanical manner.

Where the apparatus comprises a motor, which may be adapted to directly or intermittently operate the restriction device, the energy transforming device may power the motor with the energy of the second form. Suitably, the restriction device is operable to perform a reversible function and the motor is capable of reversing said function.

In accordance with another embodiment of the invention, the restriction device comprises a hydraulic restriction device, and the apparatus comprises an implantable pump for operating the hydraulic restriction device, wherein the energy transforming device supplies the energy of the second form for driving the pump. Preferably, the pump is not a plunger type of pump, but may comprise a peristaltic or membrane pump.

The energy transforming device preferably is capable of generating as the energy of the second form a current exceeding 1 μA, when transferring the energy of the first form transmitted by the energy transmission device.

The apparatus may comprise an implantable adjustment device for adjusting the restriction device to change the restriction of the urine passageway. In accordance with a first alternative the adjustment device is adapted to mechanically adjust the restriction device. In accordance with a second alternative the adjustment device is adapted to hydraulically adjust the restriction device by using implanted hydraulic means. Such hydraulic means may not use hydraulic fluid of the kind having a viscosity that substantially increases when exposed to heat or a magnetic field.

The apparatus of the present invention is not limited to the use of visible light for the wireless transmission of energy. Thus, in accordance with a broad aspect of the invention, the energy transmission device transmits energy by at least one wireless signal, preferably containing radiant energy.

The wireless signal may comprises a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined. Alternatively, the wave signal may comprise a sound wave signal, such as an ultrasonic signal. Generally, the wireless signal may comprise a digital, analog or a digital and analog signal.

The energy of the first form transmitted by the energy transmission device may comprise an electric or magnetic field transmitted in pulses, for example digital pulses. Furthermore, the energy transforming device may transform the energy of the first form, which may comprise polarized energy, into a direct current, pulsating direct current, a combination of a direct and pulsating direct current, an alternating current or a combination of a direct and alternating current. Alternatively, the energy of the first form may comprise kinetic energy.

The energy of the second form may comprise a frequency, amplitude or frequency and amplitude modulated analog, digital or combined analog and digital signal. The restriction device may be non-inflatable, i.e. with no hydraulic fluid involved for the adjustments of the restriction device. This eliminates problems with fluid leaking from the restriction device.

The apparatus suitably comprises implantable electric conductors connected to the energy transforming device, whereby the energy transforming device is capable of supplying an electric current, such as direct current, a pulsating direct current, a combination of a direct and pulsating direct current, an alternating current or a combination of a direct and alternating current, via the conductors. Furthermore, the energy transforming device may be capable of supplying a frequency, amplitude, or frequency and amplitude modulated analog, digital, or a combination of analog and digital signal, which is used in connection with control of the restriction device. In accordance with a main embodiment of the invention, the apparatus comprises an implantable operation device for operating the restriction device and a control device for controlling the operation device, wherein the energy transforming device powers the operation device with the energy of the second form. The operation device preferably comprises a motor, for example an electric linear motor or an electric rotary motor that is controlled by the control device to rotate a desired number of revolutions. Optionally, an implantable gearing may be connected to the motor. The electric motor may have electrically conductive parts made of plastics. Alternatively, the motor may comprise a hydraulic or pneumatic fluid motor, wherein the control device controls the fluid flow through the fluid motor. Motors currently available on the market are getting smaller and smaller. Furthermore, there is a great variety of control methods and miniaturized control equipment available. For example, the number of revolutions of a rotary motor may be analyzed by a Hall-element just a few mm in size.

In accordance with another embodiment of the invention, the restriction device comprises hydraulic means and the operation device is adapted to conduct a hydraulic fluid in the hydraulic means. The operation device comprises a fluid conduit connected to the hydraulic means of the restriction device, and a reservoir for fluid, wherein the reservoir forms part of the conduit. The reservoir may form a fluid chamber with a variable volume, and the operation device may be adapted to distribute fluid from the chamber to the hydraulic means of the restriction device by reduction of the volume of the chamber and to withdraw fluid from the hydraulic means to the chamber by expansion of the volume of the chamber. The operation device suitably comprises an implantable motor used for reducing and expanding the volume of the chamber. Also, the operation device may comprise a pump for pumping the hydraulic fluid in the hydraulic means of the restriction device. All of the hydraulic components involved are preferably devoid of any non-return valve. This is of great advantage, because with valves involved there is always a risk of malfunction due to improperly working valves, especially when long time periods passes between valve operations.

The control device may be adapted to reverse the operation device by shifting polarity of the energy of the second form. Where the operation device comprises an electric motor the energy of the second form suitably comprises electric energy.

In accordance with yet another embodiment of the invention, the restriction device is operable to perform a reversible function, such as enlarging and restricting the urine passageway, and there is a reversing device implanted in the patient for reversing the function performed by the restriction device. Such a reversing function preferably involves enlarging and restricting the urine passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch it may be operable by the energy of the second form. In this case, the control device suitably controls the operation of the switch by shifting polarity of the energy of the second form supplied to the switch. The switch may comprise an electric switch and the source of energy may supply electric energy for the operation of the switch. In accordance with an advantageous embodiment of the invention, the apparatus further comprises an energy storage device implanted in the patient for storing the energy of the second form and for supplying energy in connection with the operation of the restriction device. The implanted energy storage device preferably comprises an electric source of energy, such as an accumulator, a rechargeable battery or a combination of an accumulator and rechargeable battery.

The apparatus may further comprise a switch implantable in the patient for switching the operation of the restriction device and a source of energy implantable in the patient. This embodiment is particularly suited for applications where the energy transmission efficiency of the apparatus is insufficient, i.e. where the implanted restriction device is to perform more advanced operations. Such a source of energy preferably is a battery. Alternatively, the source of energy is an accumulator that also may store the energy of the second form. In accordance with a first alternative, the switch is operated by the energy of the second form supplied by the energy storage device to switch from an off mode, in which the source of energy is not in use, to an on mode, in which the source of energy supplies energy for the operation of the restriction device. In this case, the implanted source of energy may comprise a battery, preferably having a lifetime of at least 10 years, or an accumulator. However, other kinds of sources are also conceivable, such as a nuclear source of energy or a chemical source of energy (fuel cells). In accordance with a second alternative, the apparatus further comprises a remote control for controlling the supply of energy of the implanted source of energy, wherein the switch is operated by the energy of the second form supplied by the energy storage device to switch from an off mode, in which the remote control is prevented from controlling the source of energy and the source of energy is not in use, to a standby mode, in which the remote control is permitted to control the source of energy to supply energy for the operation of the restriction device.

In accordance with a third alternative, the energy storage device is omitted, wherein the switch is operated by the energy of the second form supplied by the energy transforming device to switch from an off mode, in which the remote control is prevented from controlling the source of energy and the source of energy is not in use, to a standby mode, in which the remote control is permitted to control the source of energy to supply energy for the operation of the restriction device. In accordance with a fourth alternative, also the remote control is omitted, wherein the switch is operated by the energy of the second form supplied by the energy transforming device to switch from an off mode, in which the source of energy is not in use, to an on mode, in which the source of energy supplies energy for the operation of the restriction device. Where applicable, in the described embodiments the switch may switch when the energy transmission device is transmitting wireless energy, preferably while the transferred energy of the second form is stabilized by an implanted capacitor, which may temporarily (for a few seconds) store the energy of the second form.

In the above noted third and fourth alternatives, the energy transmission device may be substituted for the energy transforming device, whereby the switch is operated by the energy of the first form. The switch mentioned above may comprise an electronic switch or, where applicable, a mechanical switch.

The advantage of using a switch above all is increased control safety; i.e. interfering signals in the patient's surroundings cannot affect the implanted restriction device. Furthermore, the lifetime of the implanted source of energy will be significantly prolonged, since the energy consumption of the apparatus will be reduced to a minimum. During the above-mentioned standby mode, the remote control uses energy from the implanted source of energy. By means of the energy transmission device energy may be transmitted to activate the switch to connect the implanted source of energy only when energy is required in connection with the operation of the restriction device.

All of the above embodiments may be combined with at least one implantable sensor for sensing at least one physical parameter of the patient, wherein the control device may control the restriction device in response to signals from the sensor. For example, the sensor may comprise a pressure sensor for directly or indirectly sensing the pressure against the restriction device, human tissue or in the urine passageway, or the pressure against the urethra or the lower part of the urine bladder. The control device may comprise an internal control unit implanted in the patient for, preferably directly, controlling the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to further restrict the urine passageway in response to the sensor sensing that the patient is lying, or enlarge the urine passageway in response to the sensor sensing an abnormally high pressure against the restriction device.

Alternatively, the control device may comprise an external control unit outside the patient's body for, suitably directly, controlling the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor. An external data communicator may be provided outside the patient's body and an internal data communicator may be implanted in the patient for communicating with the external communicator. The internal communicator may feed data related to the patient, or related to the restriction device, back to the external communicator. Alternatively or in combination, the external communicator may feed data to the internal communicator. The internal communicator may suitably feed data related to at least one physical signal of the patient.

The apparatus may further comprise an implantable programmable control unit for controlling the restriction device, preferably over time in accordance with an activity schedule program. This will advance the apparatus and make possible an adaptation of the apparatus to the individual patients.

Many of the above embodiments are suitably remote controlled. Thus, the apparatus advantageously comprises a wireless remote control transmitting at least one wireless control signal for controlling the restriction device. With such a remote control it will be possible to adapt the function of the apparatus to the patient's need in a daily basis, which is beneficial with respect to the treatment of the patient. The control signal may comprise a frequency, amplitude or frequency or amplitude modulated signal. Furthermore, the control signal may comprise an analog or a digital signal, or a combination of an analog and digital signal. The wireless remote control may be capable of obtaining information on the condition of the implanted restriction device and of controlling the restriction device in response to the information. Also, The remote control may be capable of sending information related to the restriction device from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient.

The wireless remote control may transmit a carrier signal for carrying the control signal, wherein the carrier signal is frequency, amplitude or frequency and amplitude modulated. The carrier signal may also comprise digital, analog or a combination of digital and analog signals. Such signals may comprise wave signals. Also the control signal used with the carrier signal may be frequency, amplitude or frequency and amplitude modulated, and be digital, analog or combined digital and analog. The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

The energy transmission device may function different from or similar to the energy transforming device. For example, the energy transmission and transforming devices function differently when the energy transmission device comprises a coil used for transmitting the energy of the first form and the energy transforming device comprises an electrical junction element for transforming the transmitted energy into the energy of the second form. The energy transmission and transforming devices function similar to each other when the energy transmission device comprises a coil used for transmitting the energy of the first form and the energy transforming device also comprises a coil for transforming the transmitted energy into the energy of the second form. In accordance with an alternative embodiment of the invention, the apparatus comprises an activatable source of energy implantable in the patient, wherein the source of energy is activated by wireless energy transmitted by the energy transmission device, to supply energy which is used in connection with the operation of the restriction device.

The implantable restriction device suitably is embedded in a soft or gel-like material. For example, a silicone material having hardness less than 20 Shore.

All the above described various components, such as the motor, pump and capacitor, may be combined in the different embodiments where applicable. Also the various functions described in connection with the above embodiments—may be used in different applications, where applicable. All the various ways of transferring, transforming and controlling energy presented in the present specification may be practiced by using all of the various components and solutions described.

The present invention also provides an implanting method, comprising the steps of providing a urinary incontinence treatment apparatus described above, cutting an opening in a patient's mucosa in an orifice of the patient's body, and implanting the energy transforming device in the patient's body through the opening.

There is also provided a laparascopical implanting method, in accordance with a first alternative, comprising the steps of providing a urinary incontinence treatment apparatus as described above, placing at least two laparascopic cannula within a patient's body, and implanting the energy transforming device in the patient's body by using the at least two laparascopic cannula. In accordance with a second alternative there is provided a laparoscopic surgical method of implanting a urinary incontinence treatment apparatus, comprising the steps of laparoscopically placing a restriction device of the apparatus through the abdomen or thorax of a patient, placing at least two laparoscopic trocars within the patient's body, using at least one dissecting tool inserted through the laparoscopic trocar, introducing the restriction device through the trocar, and placing the restriction device in engagement with the urethra or urine bladder to create a restricted urine passageway. The method may further comprise implanting an energy transforming device of the apparatus, for example subcutaneously, in the abdomen, thorax or cephalic region, or other locations in the patient's body.

The method may further comprise postoperatively adjusting the restricted urine passageway in a non-invasive procedure.

The present invention also provides a method of treating a human or animal having chronic urinary incontinence comprising: (a) Surgically implanting in the human or animal a restriction device engaging the human's or animal's urine bladder or urethra, to form a restricted passageway in the urethra or urine bladder.
(b) Surgically implanting in the human or animal an operation device, which can adjust the restricted passageway in response to supplied energy. And, (c) in a non-invasive post-operative procedure, from time to time, supplying energy to the operation device so as to enlarge the restricted passageway to allow urine to readily pass through. In the method (c) may be practiced several times (e.g. 2-10) a day.

### AE: Hydraulic Urinary Incontinence Treatment Apparatus

The present aspect relates to a urinary incontinence treatment apparatus, comprising an adjustable restriction device adapted to engage the urethra or urine bladder of a patient, who suffers from urinary incontinence, to form a restricted urine passageway in the urethra or urine bladder. An implantable adjustment device is provided for adjusting the restriction device to restrict the urethra or urine bladder to close the urine passageway, or release the urethra or urine bladder to open the urine passageway, when the restriction device is implanted in the patient. An implantable operation device is provided for operating the adjustment device. Urine incontinence is a widespread problem. Many people are helped through training of the muscles in the pelvic floor but too many have severe problems with urine leakage. Many different solutions to this problem have been tried. There is a prior manually operated sphincter system connected to an elastic reservoir/pump placed in the scrotum or in the region of the labia majora. A disadvantage of this system is that over time hard fibrosis is developed around the reservoir which may cause malfunction of pumping components and it is rather complicated to manually squeeze the elastic reservoir when urinating, especially woman can get their fingers wet. Thus, the created fibrosis will sooner or later become a hard fibrotic layer which may make it difficult to pump the reservoir. Yet a further disadvantage is that the use of hydraulic fluid always entails a risk of fluid leaking from the prosthesis. Furthermore, it is a rather complicated task to manually pump the reservoir when needing to urinate.

A prior hydraulic device which is made for compressing the urethra is disclosed in U.S. Pat. No. 5,520,606. Prosthetic sphincters with an inflatable cuff which surrounds the urethra or encloses it on two sides are disclosed in for example U.S. Pat. Nos. 4,571,749 and 4,222,377. U.S. Pat. No. 4,969,474 discloses a hydraulic method for treating both men and women with urinary incontinence problem in the same way. The apparatus of the U.S. Pat. No. 4,969,474 includes a reservoir containing fluid and an inflatable compression device that will compress urethra without risking tissue loss or necrosis to occur. An artificial hydraulically operated urethral sphincter employing an external magnet to achieve closure of the urethral cuff is disclosed in U.S. Pat. No. 5,562,598. A prior mechanical prosthetic sphincter, disclosed in. U.S. Pat. No. 4,619,245 comprises a manually controllable actuating component for implanting at a convenient location in the patient's body.

A prime object of the present invention is to provide a urinary incontinence treatment apparatus, which does not require manual manipulation of a combined reservoir and pump mechanism placed in the scrotum or in the region of the labia majora of the patient.

Another object is to provide an urinary incontinence treatment apparatus, which does not require complicated surgery.

Yet another object is to provide an urinary incontinence treatment apparatus, which may be conveniently controlled by the patient with a remote control.

These objects are obtained by an apparatus of the kind described initially characterized in that the operation device comprises a powered operation device and/or a servo means.

The expression “powered” should be understood as energized with everything without manual force, preferably electric energy. In other words, the adjustment device is operated in a non-manual manner. The expression “non-manual manner” should be understood to mean that the adjustment device is not operated by manually touching subcutaneously implanted components of the apparatus or not manipulated by touching the skin of the patient. Thus, as opposed to prior practice when treating urine incontinence, the adjustment device is not operated by manual forces, such as by manually compressing a fluid containing balloon implanted in the scrotum or in the region of labia majora. Of course, manual manipulation of a subcutaneous start button or the like for activating the powered operation device is permitted within the scope of the present invention. Alternatively, or in combination with a powered operation device, the servo means may be used, which enables manual manipulation without need for strong manipulation forces. The servo means may comprise hydraulic means, electric control means, magnetic means, or mechanical means, which may be activated by manual manipulating means. Using a servo system will save the use of force when adjusting the adjustment device, which may be of importance in many applications.

The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible.

In accordance with a main embodiment of the invention, the apparatus comprises a reservoir, preferably containing a predetermined amount of hydraulic fluid, also implantable in the patient, wherein the operation device, suitably electrically powered, operates the adjustment device by using the hydraulic fluid of the reservoir.

The adjustment device may comprise an expandable cavity in the restriction device, wherein the urethra or urine bladder is squeezed upon expansion of the cavity and released upon contraction of the cavity. In this embodiment the operation device is adapted to distribute hydraulic fluid from the reservoir to expand the cavity, and from the cavity to the reservoir to contract the cavity.

A fluid distribution tube may readily be connected between the reservoir and the cavity in a manner so that the tube does not interfere with other implanted components of the apparatus.

Preferably, the reservoir defines a chamber for the predetermined amount of fluid and the operation device changes the volume of the chamber. The operation device suitably comprises first and second wall portions of the reservoir and is adapted to provide relative displacement between the first and second wall portions of the reservoir, in order to change the volume of the chamber.

The operation device may be adapted to provide said relative displacement in response to the pressure in the reservoir. Suitably, the operation device comprises a pressure controlled hydraulic operation device. For safety, an alarm may be provided for generating an alarm signal in response to the lapse of a predetermined time period during which the pressure controlling the hydraulic operation device exceeds a predetermined high value.

Suitably, the operation device is adapted to distribute fluid from the reservoir to the cavity of the restriction member in response to a predetermined first displacement of the first wall portion of the reservoir relative to the second wall portion of the reservoir and may distribute fluid from the cavity to the reservoir in response to a predetermined second displacement of the first wall portion relative to the second wall portion.

The first and second wall portions of the reservoir may be displaceable relative to each other by a magnetic, hydraulic, or electric power means, such as an electric motor. In this embodiment no pump is used, only the volume of the reservoir is varied. This is of great advantage compared to the solution described below when the operation device comprises a pump used to pump fluid between the reservoir and the adjustment device because there is no need for a nonreturn valve and it is still possible to have fluid going both to and from the reservoir. Thus, the significant risk of malfunction when using such a non-return valve implanted in the patient is eliminated. The operation device may comprise hydraulic means and a fluid conduit extending between the hydraulic means and the adjustment device. The hydraulic means and conduit are devoid of any non-return valve. The reservoir may form part of the conduit and a fluid chamber with a variable volume. The operation device may distribute fluid from the fluid chamber to the adjustment device by reduction of the volume of the chamber and withdraw fluid from the adjustment device by expansion of the volume of the chamber. The operation device preferably comprises a motor for moving a movable wall of the reservoir for changing the volume of the chamber. Any kind of motor could be used for the different operations as well as wireless remote solutions for controlling the operations. The restriction device preferably is operable to perform a reversible function and accordingly there is a reversing device implantable in the patient for reversing the function performed by the restriction device. Such a reversing function preferably involves enlarging and restricting the urine passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox. Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch.

Where the operation device comprises a motor, the reversing device is adapted to reverse the motor.

In accordance with another particular embodiment of the invention, the operation device comprises a pump for pumping fluid between the reservoir and the adjustment device. A mechanical solution is proposed in which it is possible to pump fluid from the reservoir to the adjustment device and vice versajust by pushing an activation member in one direction. The pump preferably comprises a first activation member for activating the pump to pump fluid from the reservoir to the adjustment device, and a second activation member for activating the pump to pump fluid from the adjustment device to the reservoir. At least one of the first and second activation members may be operable by manual manipulation, preferably to permit manual pushing, pulling or rotation thereof in one direction, or by a device powered magnetically, hydraulically, or electrically (e.g. by an electric motor), or be operable by a combination of these methods. Suitably, at least one of the activation members may be adapted to operate when subjected to an external pressure exceeding a predetermined magnitude.

Another alternative is a pump pumping in only one direction and an adjustable valve to change the direction of fluid to either increase or decrease the amount of fluid in the reservoir. This valve may be manipulated either manually, mechanically, magnetically, or hydraulically.

The main embodiment described above including the reservoir may alternatively be equipped with a servo means comprising a reverse servo. The term “reverse servo” is to be understood as a mechanism that transfers a strong force acting on a moving element having a short stroke into a weak force acting on another moving element having a long stroke; i.e. the reverse function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device. The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the normal servo means and the specific reverse servo may be used in connection with all of the various components and solutions described in the present specification.

Thus, the reverse servo may be adapted to provide relative displacement between the first and second wall portions of the reservoir, suitably in response to the pressure in the reservoir, in order to change the volume of the chamber of the reservoir.

Generally, the servo means, including the reverse servo, comprises a pressure controlled servo means. The alarm mentioned above may alternatively be adapted to generate an alarm signal in response to the lapse of a predetermined time period during which the pressure controlling the servo means exceeds a predetermined high value.

The reverse servo may comprise magnetic means, electric means or manual manipulation means or a combination thereof. Preferably, however, the reverse servo comprises hydraulic means.

In accordance with a particular embodiment of the invention, the reverse servo further comprises a servo reservoir defining a chamber containing servo fluid, and the operation device comprise first and second wall portions of the servo reservoir, which are displaceable relative to each other to change the volume of the chamber of the servo reservoir. The first and second wall portions of the servo reservoir may be displaceable relative to each other by magnetic means, hydraulic means, or electric control means.

Where the reverse servo comprises hydraulic means it may further comprise a fluid supply reservoir connected to the servo reservoir in a closed system and containing a further predetermined amount of fluid. The fluid supply reservoir defines a chamber for the further predetermined amount of fluid and the operation device is adapted to change the volume of the chamber and thereby control the amount of fluid in the servo reservoir. The fluid supply reservoir comprises first and second wall portions, which are displaceable relative to each other to change the volume of the chamber of the fluid supply reservoir. Suitably, the fluid supply reservoir increases the amount of fluid in the servo reservoir in response to a predetermined first displacement of the first wall portion of the fluid supply reservoir relative to the second wall portion of the fluid supply reservoir and decreases the amount of fluid in the servo reservoir in response to a predetermined second displacement of the first wall portion of the fluid supply reservoir relative to the second wall portion of the fluid supply reservoir.

In accordance with an embodiment of the invention, the adjustment device comprises a hydraulic adjustment device, and an implantable reservoir containing a predetermined amount of hydraulic fluid and a conduit providing fluid connection between the reservoir and the hydraulic adjustment device are provided. The operation device is adapted to operate the hydraulic adjustment device by distributing hydraulic fluid through the conduit between the reservoir and the hydraulic adjustment device, wherein the conduit and hydraulic adjustment device are devoid of any non-return valve to permit free flow of hydraulic fluid in both directions in the conduit. Preferably, the reservoir forms a fluid chamber with a variable volume, and the operation device is adapted to distribute fluid from the chamber to the adjustment device by reduction of the volume of the chamber and to withdraw fluid from the adjustment device by expansion of the volume of the chamber. The operation device may comprise a motor or a pump. Alternatively, the operation device may comprise a movable wall of the reservoir for changing the volume of the chamber. For example, the operation device may be adapted to change the volume of the chamber by moving the movable wall in response to the pressure in the chamber.

In the above embodiments including a reservoir for hydraulic fluid an injection port may be provided for subcutaneous implantation in the patient to be in fluid communication with the chamber of the reservoir. The injection port may be integrated in the reservoir. Such an injection port may be provided for enabling, normally single, once-and-for-all, calibration of the amount of fluid in the hydraulic system used.

In the various embodiments hereinafter described the restriction device generally forms an at least substantially closed loop. However, the restriction device may take a variety of different shapes, such as the shape of a square, rectangle or ellipse. The substantially closed loop could for example be totally flat, i.e. thin as seen in the radial direction. The shape of restriction device may also be changed during use, by rotation or movements of the restriction device in any direction. A physical lumen, like the urethra or urine bladder, often is easier to restrict by contracting two opposite sidewalls of the lumen against each other. Thus, the restriction device may be designed to perform such a contracting effect of the opposite walls of the urethra or urine bladder. Either mechanical or hydraulic solutions may be employed to operate the restriction device. Alternatively, the restriction device may comprise an adjustable cuff, a clamp or a roller for bending or rotating the urethra or urine bladder to close its passageway. Such a cuff, clamp or roller may also be utilized for squeezing the urethra or urine bladder against human material inside the body of the patient, for example the sacral bone of the patient, or against implanted structures of the apparatus. The bending or rotating members may take any shape and be either hydraulic or non-inflatable.

Preferably the restriction device comprises an elongated restriction member and forming means for forming the restriction member into at least a substantially closed loop around the urethra or urine bladder, wherein the loop defines a restriction opening, whereby the adjustment device adjusts the restriction member in the loop to change the size of the restriction opening.

Advantageously, the forming means may form the restriction member into a loop having a predetermined size. Alternatively, the forming means may form the restriction member into a loop having a size selected from several predetermined sizes.

The adjustment device may change the size of the restriction opening such that the outer circumferential confinement surface of the restriction member either is changed or is unchanged. The elongated restriction member may be flexible, for example take the shape of a belt or cord, and the adjustment device may pull a first portion of the flexible restriction member from a second portion of the flexible restriction member opposite the first portion in the loop to squeeze the urethra or urine bladder between the opposite lengths of the elongated flexible restriction member to restrict the urine passageway. The restriction member may be non-inflatable, and the adjustment device may mechanically adjust the restriction member in the loop.

The adjustment device may mechanically or hydraulically adjust the restriction device. In the embodiments described the adjustment device may either mechanically or hydraulically adjust the restriction device, where applicable. It should be noted that the operation device might mechanically or hydraulically operate the adjustment device irrespectively of whether the adjustment device is adapted to adjust the restriction device mechanically or hydraulically.

In accordance with an embodiment of the invention, the restriction device comprises at least two elements on opposite or different sides of the urethra or urine bladder, and the adjustment device decreases the distance between the elements to squeeze the urethra or urine bladder between the elements, thereby restricting the urine passageway. It is also possible to use only one element and squeeze the urethra or urine bladder against human bone or tissue. The elements above may as well as all the restriction members mentioned in this application be everything from rigid to soft.

In accordance with an alternative, the restriction device bends or rotates a portion of the urethra or urine bladder to restrict the urine passageway in the same. For example, the restriction device may comprise at least two-bending members, such as cylindrical or hour-glass shaped rollers, positioned on opposite or different sides of the urethra or urine bladder and displaced relative to each other along the urethra or urine bladder, and the adjustment device may move the bending members against the urethra or urine bladder to bend the latter to restrict the urine passageway. The restriction device may also rotate a portion of the urethra or urine bladder. The bending or rotating members may take any shape and be either hydraulic or non-inflatable. Alternatively, the two bending members one placed more distal than the other may be rotated in opposite directions relative to each other. With interconnecting means for example flexible bands between the bending members a restriction will occur between the bending members when they are rotated. Preferably the adjustment device is operable to adjust the restriction device to steplessly change the restriction of the urine passageway in the urethra or urine bladder.

All embodiments according to the invention may be controlled by a wireless remote control. In accordance with an advantageous embodiment of the invention, there is provided a wireless remote control for non-invasively controlling the operation device. The remote control may conveniently comprise an external hand-held remote control unit, which is manually operable by the patient to control the restriction device to squeeze and release the urethra or urine bladder. With the wireless remote control the apparatus is conveniently controlled by the patient when he so desires, which is of great advantage compared to the prior art procedures. With the remote control the apparatus is conveniently controlled to adjust the implanted restriction device to release the urine passageway when the patient wants to relieve himself or herself.

The remote control may advantageously be capable of obtaining information related to important parameters, such as the condition of the urine passageway or the pressure against the restriction device, and of commanding the operation device to operate the adjustment device to adjust the restriction device in response to obtained information. With the remote control the apparatus is conveniently controlled to adjust the implanted restriction device to open and close the urine passageway. The adjustment device may control the restriction device to steplessly change the restriction of the passageway.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter. Any known or conventional signal transmitting or signal receiving means that is suitable for use with a human or mammal patient may be provided as the signal transmitter or signal receiver.

The apparatus may further comprise an implanted energizer unit for providing energy to energy consuming implanted components of the apparatus, such as electronic circuits and/or a motor for operating the adjustment device. Where a motor is provided the control unit is adapted to power the motor with energy provided by the energizer unit in response to a control signal received from the signal transmitter. The motor may be any type of motor, such as a pneumatic, hydraulic or electric motor and the energizer unit may power the motor with pressurized gas or liquid, or electric energy, depending on the type of motor. Where the motor is an electric motor, it may power pneumatic or hydraulic equipment.

The remote control advantageously comprises wireless energy transfer device for transferring energy from outside the patient's body to energy consuming implantable components of the apparatus. The energy transfer device may comprise said energizer unit is adapted to transform energy from the control signal, as it is transmitted to the signal receiver, into electric energy. Where the operation device comprises a motor the wireless energy-transfer device is adapted to directly power the motor with transferred energy.

The energy transferred by the wireless energy transfer device preferably comprises a signal, suitably a wave signal. The energy transferred by the wireless energy transfer device may comprise an electric field or a magnetic field or a combination thereof. The signal may be analog or digital or a combination thereof. The energy transfer device may transfer the energy from the signal into a direct, pulsating direct or alternating current or a combination thereof.

Any of the above mentioned signals may comprise analog or digital pulses. The analog or digital signal may comprise a magnetic field or an electric field or a combination thereof. Where the signal is a wave signal it may comprise an electromagnetic wave signal, a sound wave signal or a carrier wave signal for a remote control signal or a combination thereof. Where a carrier signal is used it may be frequency, is amplitude or frequency and amplitude modulated.

The apparatus may comprise an implantable source of energy for powering the operation device and/or for energizing other energy consuming components of the apparatus, wherein the energy from the source of energy is releasable from outside the patient's body. Furthermore, the apparatus may comprise an energy transmission device for wireless transmission of energy of a first form and an energy transforming device implantable in the patient for transforming the energy of the first form into energy of a second form, to be supplied to the source of energy and/or other implantable energy consuming parts of the apparatus. The energy transforming device may transform the wireless energy directly or indirectly into energy different than the wireless energy for operation of the restriction device. Typically, the energy of the second form is different than the energy of the first form. The function of the energy transmission device may be different from that of the energy transforming device.

An implantable motor or pump for operating the adjustment device may be provided, wherein the energy transmission device may be adapted to transmit wireless energy in the form of a magnetic field or electromagnetic waves or field for direct power of the motor or pump, as the wireless energy is being transmitted. Suitably, the energy transmission device transmits energy by at least one signal separate from the above mentioned control signal.

An implantable stabilizer for stabilizing the energy of the first or second form may be provided. Where the energy of the second form comprises electric current, the stabilizer suitably comprises at least one capacitor.

Generally, the source of energy comprises a battery, accumulator, capacitor or a combination thereof. In accordance with an embodiment of the invention, the apparatus comprises a control device adapted to produce wireless energy for directly powering the operation device and/or for energizing other energy consuming components of the apparatus.

It should be understood that the energy consuming parts of the apparatus for example a motor or pump may be or may not be energized with the unchanged wirelessly transmitted energy as this being transmitted as well as being or not being energized with energy different than the transmitted energy for example transformed into electrical energy but still directly used for energizing the energy consuming parts of the apparatus as the transmitted energy is transmitted. Alternatively the energy consuming parts of the apparatus may be energized from a implanted source of energy or storage device, which still may be loaded with wireless energy. In all these aspects it is preferable to be able to wirelessly control the release of energy and get an feedback of the result of the performed function of the device. Direct use of transmitted energy may be unreliable without a feedback what has happened, has the energy reached its goal?

Generally, the wireless energy may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal.

Any of the above mentioned signals may comprise a wave signal including a sound wave signal, an ultrasound wave signal, an electromagnetic wave signal, an infrared light signal, a visible light signal, an ultra violet light signal, a laser light signal, a micro wave signal, a radio wave signal, an x-ray radiation signal or a gamma radiation signal.

The control device may be adapted to produce wireless energy in the form of a train of energy pulses and the energy transfer device may be adapted to intermittently transfer the train of energy pulses for direct use in connection with the energizing of. the energy consuming components of the apparatus. Alternatively, the control device may be adapted to control the energy transforming device to produce the energy of the second form in said train of energy pulses for direct use in connection with the operation of the adjustment device. The transferred energy preferably comprises electric energy. An implantable capacitor may be provided for producing the train of energy pulses.

Where a capacitor is used in any of the above described embodiments it may have a relatively low capacity, i.e. less than 0.1 μF, in order to be small and suited for implantation.

Where the operation device comprises an implantable motor or pump for operating the adjustment device, the energy transfer device may be adapted to directly power the motor or pump with transferred energy, at the same time as the energy is transferred. Where a pump is used it should not be a plunger type of pump, because a plunger pump is noisy, but may comprise a peristaltic or membrane pump.

As mentioned above the apparatus comprises a wireless remote control for non-invasively controlling the operation device, which preferably is electrically powered. Alternatively, the operation device is powered by magnetic energy, non-magnetic energy, electromagnetic energy, non-electromagnetic energy, kinetic energy, non-kinetic energy, sonic energy, non-sonic energy, thermal energy or non-thermal energy. However, the operation device may be unpowerable by permanent static magnetic energy. Any other kind of energy, such as electric, electromagnetic energy or a moving permanent magnetic energy, may be conceivable for operating the adjustment device. As a result, the implanted restriction device would not be accidentally adjusted if the patient comes close to any permanent magnet . . . . Suitably, the operation device is adapted to non-invasively operate the adjustment device.

Where the operation device comprises a hydraulic operation device it may use hydraulic fluid, the viscosity of which changes when the hydraulic fluid is exposed to energy, preferably electric energy, different than thermal energy. However, use of hydraulic fluid of the kind having a viscosity that increases when exposed to heat or a magnetic field, i.e. the hydraulic fluid would not become substantially more viscous when exposed to heat or influenced by magnetic forces, should be avoided, because external heat sources or heat from the body when the patient has fever and external magnetic sources might affect the implanted components of the apparatus.

The adjustment device is may be operable to adjust the restriction device to steplessly change the restriction of the urine passageway. Furthermore, the adjustment device may be adapted to mechanically adjust the restriction device. Alternatively, it may be adapted to hydraulically adjust the restriction device by using hydraulic means, which is devoid of hydraulic fluid of the kind having a viscosity that substantially increases when exposed to heat or a magnetic field.

In accordance with an embodiment of the invention, the apparatus comprises a control device. for controlling the restriction device. The control device may comprise an internal programmable control unit implantable in the patient and, possibly an external control unit outside the patient's body for programming the programmable internal control unit. Alternatively, the external control unit may be programmable and wirelessly control the restriction device. The control device may be adapted to produce wireless energy for directly powering the operation device and/or for energizing other energy consuming components of the apparatus.

At least one sensor for sensing at least one physical parameter of the patient may conveniently be implanted in the patient. The sensor may preferably sense as the physical parameter the horizontal position of the patient or may comprise a pressure sensor for sensing the pressure against the restriction device or the urethra or urine bladder or other important parameters. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor.

Either the internal control unit or the external control unit of the control device may suitably control the restriction device to enlarge or close the urine passageway. For safety the restriction device may enlarge or open the urine passageway in response to the sensor sensing for example an abnormally high pressure value. The internal control unit may directly control the restriction device in response to signals from the sensor. Wherever magnetic means is utilized according to the invention it may comprise a permanent magnet and a magnetic material reed switch, or other suitable known or conventional magnetic means.

Where a source of energy is used the control device suitably is operable from outside the patient's body for controlling the source of energy to release energy for use in connection with the operation of the adjustment device, when the adjustment device is implanted. The source of energy may be provided external to the patient's body, and the control device may be adapted to control the external source of energy to release wireless energy for use in connection with the operation of the adjustment device.

The control device may control the source of energy to release magnetic energy, non-magnetic energy, electromagnetic energy, non-electromagnetic energy, kinetic energy, non-kinetic energy, sonic energy, non-sonic energy, thermal energy or non-thermal energy, preferably in a non-invasive manner and for a determined time period and/or in a determined number of energy pulses.

Where the implantable components of the apparatus comprise electrical electrical components they may include at least one or a single voltage level guard. In this case, the electrical components suitably are devoid of any current detector and/or charge level detector. Furthermore, the electrical components may comprise a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

In accordance with an advantageous embodiment of the invention, the apparatus comprises an implantable switch for directly or indirectly switching the operation of the* restriction device. The switch may be operated by the energy supplied by the energy transmission device mentioned above to switch from an off mode, in which the implantable source of energy mentioned above is not in use, to an on mode, in which the source of energy supplies energy for the operation of the restriction device. In* accordance with an alternative embodiment, the above mentioned a remote control may be employed for controlling the implantable source of energy, wherein the switch is operated by the energy supplied by the energy transmission device to switch from an off mode, in which the remote control is prevented from controlling the source of energy and the source of energy is not in use, to a standby mode, in which the remote control is permitted to control the source of energy to supply energy for the operation of the restriction device. In accordance with another alternative embodiment, the switch is operated by the energy supplied by the implantable energy transforming device mentioned above to switch from an off mode, in which the source of energy is not in use, to an on mode, in which the source of energy supplies energy for the operation of the restriction device.

In accordance with yet another alternative embodiment, the switch is operated by the energy supplied by the energy transforming device to switch from an off mode, in which the remote control is prevented from controlling the source of energy and the source of energy is not in use, to a standby mode, in which the remote control is permitted to control the source of energy to supply energy for the operation of the restriction device.

Suitably, the restriction device is embedded in a soft or gel-like material, such as a silicone material having hardness less than 20 Shore.

The energy transforming device may be designed to be implanted subcutaneously or in the abdomen, thorax or cephalic region of the patient. The adjustment device may be adapted to adjust the restriction device such that the restriction device provides a predetermined contraction of the urine passageway that is satisfactory for the patient.

The adjustment device is preferably adapted to adjust the prosthesis device in a non-flux magnetic or non-thermal manner or non-viscosity changing manner, because these would give rise to unreliable function of the** device. The expression “non-viscosity changing manner” should be understood to mean that the adjustment device is not adjusted by changing the viscosity of the hydraulic fluid used.

All the above described various components, such as the motor, pump and capacitor, may be combined in the different embodiments where applicable. Also the various functions described in connection with the above embodiments may be used in different applications, where applicable. Specifically, the various remote control functions described and all the various methods for supplying energy may be used in any conceivable combination that is apparent to those skilled in the art.

The invention also provides a method for treating a patient suffering from urinary incontinence, comprising the steps of placing at least two laparascopical trocars in the body of a patient suffering from urinary incontinence, inserting a dissecting tool through the trocars and dissecting an area of the urethra or urine bladder in the abdominal or pelvic or retroperitoneal surroundings, placing at least one adjustable restriction device in the dissected area engaging the urethra or urine bladder adjusting the restriction device to normally restrict the urine passageway in the urethra or urine bladder, and adjusting the restriction device to open the urine passageway when the patient wants to relieve himself or herself. A hydraulic adjustable restriction device may be used when practicing this method, preferably in a non-manual manner, i.e. without touching subcutaneously implanted components of the apparatus.

The method may further comprise implanting a source of energy in the patient and providing a control device for controlling the source of energy from outside the patient's body to supply energy for the adjustment of the restriction device.

The present invention also provides a method for treating urinary incontinence, comprising surgically implanting in the body of a patient suffering from urinary incontinence an adjustable restriction device engaging the urethra or urine bladder to restrict the urine passageway, and when desired to allow the patient to urinate, adjusting the restriction device to temporarily release the urethra or urine bladder to open the urine passageway. The method may further comprise implanting an elongated restriction member of the restriction device around the urethra or urine bladder.

The urinary incontinence treatment apparatus may also be laparoscopicly implanted. Thus, there is provided a method comprising the steps of placing at least two laparascopical trocars in the patient's body, inserting a dissecting tool through the trocars and dissecting an area of the pelvic or abdominal or retroperitoneal surroundings and placing an operable restriction device in the dissected area, so that the restriction device engages the urethra or urine bladder to restrict the urine passageway.

### AF: Urinary Incontinence Treatment Apparatus

The present aspect relates to a urinary incontinence treatment apparatus for treatment of a patient, who suffers from urinary incontinence, comprising an adjustable restriction device implantable in the patient, for engaging a portion of the urethra or urine bladder of the patient to restrict a urine passageway therein, and an operable adjustment device adapted to mechanically adjust the restriction device to change the restriction of the urine passageway.

Urine incontinence is a widespread problem. Many people are helped through training of the muscles in the pelvic floor but too many have severe problems with urine leakage. Many different solutions to this problem have been tried. For example, there is a prior manually operated urine incontinence treatment apparatus having an artificial hydraulic sphincter device engaging the urethra and connected to an elastic reservoir implanted in the scrotum or in the region of the labia major. A disadvantage of this prior apparatus is that over time hard fibrosis is developed around the reservoir that may cause malfunction of pumping components. Furthermore, it is a rather complicated task, to manually squeeze the elastic implanted reservoir to pump hydraulic fluid to open the sphincter device when the patient needs to urinate. In particular women can get their fingers wet. The created fibrosis will sooner- or later-become a hard fibrotic layer that may make it even more difficult to pump the reservoir. Yet a further disadvantage is that the use of hydraulic fluid always entails a risk of fluid leaking from implanted hydraulic components.

A prior hydraulic apparatus designed to compress the urethra is disclosed in U.S. Pat. No. 5,520,606. Prosthetic sphincters with an inflatable cuff that surrounds the urethra or encloses it on two sides are disclosed in for example U.S. Pat. Nos. 4,571,749 and 4,222,377. U.S. Pat. No. 4,969,474 discloses a hydraulic method for treating both men and women with urinary incontinence problems in the same way. The apparatus of U.S. Pat. No. 4,969,474 includes a reservoir containing fluid and an inflatable compression means designed to compress urethra without risking tissue loss or necrosis to occur. An artificial hydraulically operated urethra sphincter employing an external magnet to achieve closure of the urethra cuff is disclosed in U.S. Pat. No. 5,562,598.

A prior mechanical prosthetic sphincter disclosed in U.S. Pat. No. 4,619,245 comprises a manually controllable actuating component for implanting at a convenient location in the patient's body.

A prime object of the present invention is to provide a urinary incontinence treatment apparatus in which the risk of liquid leakage within the patient's body is substantially reduced or completely eliminated.

A further object is to provide a urinary incontinence treatment apparatus, which does not require manual manipulation of a combined reservoir/pump mechanism in the scrotum or labia major region of the patient. These objects are obtained by an apparatus of the kind stated initially characterized in that the adjustment device is inoperable by permanent static magnetic energy. Any other kind of energy, such as electric, electromagnetic energy or a moving permanent magnetic energy, may be conceivable for operating the adjustment device. As a result, the implanted restriction device would not be accidentally adjusted if the patient came close to any permanent magnet. Conveniently, the adjustment device may adjust the restriction device in a non-manual or non-invasive manner. The expression “non-manual manner” should be understood to mean that the restriction device is not adjusted by manually touching subcutaneously implanted components of the apparatus. Furthermore, the adjustment device may adjust the restriction device in a non-thermal manner or in a non-magnetic manner, i.e. magnetic forces may not be involved at all when adjusting the restriction device. The restriction device preferably is adapted to control, suitably steplessly, the cross-sectional area of the urine passageway, i.e. to open and close the urine passageway.

Generally, the adjustment device is powered for adjusting the restriction device. As opposed to prior art urinary incontinence treatment devices, the adjustment device is not operated by manual forces, such as by manually compressing a fluid containing balloon implanted in the scrotum. Instead the apparatus may further comprise a powered operation device for operating the adjustment device. It would even be possible for patients with paresis of the bladder to have a feed-back signal from the system informing the patient when the bladder is full, for example by using some kind of pressure, tension or level sensor. In the various embodiments hereinafter described the restriction device generally is adapted to form an at least substantially closed loop . . . around said portion of the urethra or urine bladder. However, the restriction device may take a variety of different shapes, such as the shape of a square, rectangle or ellipse. The substantially closed loop could for example be totally flat, i.e. thin as seen in the radial direction. The shape of the restriction device may also be changed during use, by rotation or movements of the restriction device in any direction.

A physical lumen, such as the urethra or urine bladder, is often easier to restrict by contracting at least two opposite or different sidewalls of the lumen against each other. Either mechanical or hydraulic solutions may be employed to operate the restriction device. Alternatively, the restriction device may comprise an adjustable cuff, a clamp or a roller for bending the urethra or urine bladder to restrict the urine passageway therein. Such a cuff, clamp or roller may also be utilized for squeezing the urethra or urine bladder against human material inside the body of the patient for an example the sacral bone of the patient.

Preferably, the restriction device comprises an elongated restriction member and forming means for forming the restriction member into at least a substantially closed loop around said portion of the urethra or urine bladder, wherein the loop defines a restriction opening, whereby the adjustment device adjusts the restriction member in the loop to change the size of the restriction opening.

The restriction device may be designed for implantation in the abdomen or pelvic region or retroperitoneum of the patient and preferably may engage the urethra or urine bladder. The adjustment device may be incorporated in the restriction device as well as controlled by hydraulic means.

In accordance with a—preferred first adjustment principle, the adjustment device mechanically adjusts the longitudinal extension of the elongated restriction member in a loop form.

In a preferred embodiment utilizing the first adjustment principle, the restriction member comprises a main portion and two elongated end portions, and the adjustment device establishes longitudinal relative displacement between the end portions of the restriction member, so that the size of the restriction opening is adjusted. The forming means may comprise any suitable known or conventional device capable of practicing the desired function, such as a spring material forming the elongated restriction member into the loop, so that the restriction opening has a predetermined size, and the adjustment device may adjust the restriction member against the spring action of the spring material. In other words, the restriction member may comprise a spring cli. The spring material may be integrated in the restriction member.

Preferably, the adjustment device comprises a movement transferring member, suitably a drive wheel, in engagement with at least one of the end portions of the restriction member and operable to displace the one end portion relative to the other end portion of the restriction member. The drive wheel may advantageously be in engagement with both of the end portions of the restriction member and be operable to displace the end portions relative to each other. An elongated flexible drive shaft may be operatively connected to the drive wheel, for transferring manual or—motor generated power from a location remote from the restriction member. In its simplest embodiment, the drive wheel may comprise a pulley in frictional engagement with the restriction member. As an alternative, a gear rack may be—formed on at least one of the end portions of the restriction member and the drive wheel may comprise a gear wheel in mesh with the gear rack. Other suitable known or conventional mechanisms may also or alternatively be used as the adjustment device.

The movement transferring member may alternatively comprise at least one cylinder and a piston, which is movable therein and is connected to one of the end portions of the restriction member, the piston being operable to longitudinally displace the one end portion of the restriction member relative to the other end portion of the restriction member. Alternatively, the movement transferring means may comprise two interconnected cylinders and two pistons in the respective cylinders connected to the end portions, respectively, of the restriction member, the pistons being operable to longitudinally displace the end portions of the restriction member relative to each other. Other known or conventional devices also or alternatively can be used as the movement transferring member.

A motor, which is fixed relative to the main portion of the restriction member and has a rotating drive shaft operatively connected to the movement transferring member, may be positioned relative to the elongated restriction member such that the drive shaft extends transverse thereto. Alternatively, the motor may be positioned relative to the elongated restriction member such that the drive shaft extends substantially tangentially to the loop of the restriction member.

In another embodiment utilizing the first adjustment principle, the elongated restriction member is longitudinally resilient and the adjustment device comprises a contraction device for longitudinally contracting the resilient restriction-member. Preferably, the elongated restriction member comprises a substantially non-resilient main portion and an end portion forming an elongated helical spring, which is contractible by the contraction device. The contraction device may suitably comprise an elongated flexible pulling member connected to the main portion of the restriction member and extending through the helical spring to contract the helical spring against an arresting member, which is fixed relative to the main portion of the restriction member.” The pulling member may extend in an elongated tube joined at one end thereof to the arresting member, so that a motor remote from the restriction member may be attached to the other end of the elongated tube and pulls the pulling member through the tube to contract the helical spring.

In yet another embodiment utilizing the first adjustment principle, the elongated restriction member comprises an elongated helical spring having a free end, and a body to which the spring is non-rotatably secured at its opposite end. The adjustment device rotates the helical spring in one direction to enlarge the coils of the helical spring to longitudinally contract the spring and to rotate the spring in the opposite direction to reduce the size of the coils of the spring to longitudinally extend the spring. As a preferred alternative, the restriction member comprises a further elongated helical spring having a free end and non-rotatably secured to the body at its opposite end, and the adjustment device comprises a drive shaft having two opposite end portions connected to the springs, respectively, at their free ends, the helical coils forming left and right hand helices, respectively. The adjustment device may alternatively comprise a gearing having an input shaft and two opposite aligned output shafts connected to the helical springs, respectively, at their free ends, the—Input—shaft being connected to the output shafts so that the output shafts rotate in the opposite directions upon rotation of the input shaft, the helical coils forming the same helices.

In accordance with a second adjustment principle, the adjustment device mechanically adjusts the restriction member so that at least a portion of a radially innermost circumferential confinement surface formed by the restriction member is substantially radially displaced. in one embodiment utilizing the second adjustment principle, the restriction member comprises an elongated voltage responsive element forming part of the confinement surface and capable of. bending into a bow in response to a voltage applied across the element, the radius of curvature of the bow being adjustable by changing the level of the voltage.

In another embodiment utilizing the second adjustment principle, the adjustment device changes the diameter of an elastic annular element of the restriction member, which forms the confinement surface. Preferably, the forming means comprises a substantially rigid outer annular element coaxially surrounding the elastic annular element, and the adjustment device comprises means for pulling the elastic annular element radially outwardly towards the outer annular element to expand the elastic annular element. For example, the pulling means may comprise a plurality of threads secured to the elastic annular element along the circumference thereof and running from the elastic annular element via guide members attached to the outer annular element.

In yet another embodiment utilizing the second adjustment principle, the forming means comprises a substantially rigid outer annular element, and the restriction member comprises an elongated helical spring extending internally along the outer annular element and contacting the latter. The helical spring forms part of the circumferential confinement surface and has a free end. The restriction member further comprises a body to which the spring is non-rotatably secured at its opposite end. The adjustment device rotates the helical spring in one direction to enlarge the coils of the spring to contract the circumferential confinement surface and rotates the spring in the opposite direction to reduce the size of the coils of the spring to expand the circumferential confinement surface. As an alternative, which is preferred, the restriction member comprises two elongated helical springs forming part of the circumferential confinement surface and connected to the body of the restriction member. The adjustment device rotates each spring in one direction to enlarge the coils of the spring to contract the circumferential confinement surface and rotates the spring in the opposite direction to reduce the size of the coils of the spring to expand the circumferential confinement surface.

In accordance with a third adjustment principle, the restriction member comprises at least two separate elements, at least one of which is pivoted so that it may turn in a plane in which the restriction member extends, and the adjustment device turns the pivoted element to change the size of the restriction opening. Preferably, the restriction member comprises a plurality of separate pivoted elements disposed in series, each pivoted element being turnable in the plane, and the adjustment device turns all of the pivoted elements to change the size of the restriction opening. For example, the pivoted elements may comprise lamellae arranged like the conventional adjustable aperture mechanism of a camera.

In accordance with a fourth adjustment principle, the adjustment device folds at least two foldable frame elements of the restriction member towards each other. Preferably, the foldable frame elements comprise two substantially or partly semi-circular frame elements which are hinged together so that the semi-circular elements are swingable relative to each other from a fully open state in which they form part of a circle to a fully folded state in which they form part of a semi-circle. The same principle may be used with the swingable parts mounted together in one end and not in the other end.

Alternatively, the restriction device may comprises two preferably rigid articulated clamping elements positioned-on opposite or different sides of the urethra or urine bladder, and the adjustment device turns the clamping elements toward each other to clamp the urethra or urine bladder between the clamping elements, thereby restricting the urine passageway in the urethra or urine bladder.

In accordance with a fifth adjustment principle, the adjustment device turns the restriction member around a longitudinal extension thereof, the elongated restriction member being elastic and varying in thickness as seen in a cross-section thereof. Suitably, the elongated restriction member comprises an elastic belt.

In accordance with a sixth adjustment principle, the adjustment device changes the size of the restriction opening such that the outer circumferential confinement surface of the restriction member is changed.

In accordance with a seventh adjustment principle, the adjustment device changes the size of the restriction opening such that the outer circumferential confinement surface of the restriction member is unchanged. In accordance with an eighth adjustment principle, the elongated restriction member may be flexible, and the adjustment device pulls a first portion of the flexible restriction member from a second portion of the flexible restriction member opposite the first portion in the loop to squeeze the urethra or urine bladder between the opposite lengths of the elongated flexible restriction member to restrict the urine passageway in the urethra or urine bladder In accordance with a ninth adjustment principle, the restriction device comprises at least two elements on opposite or different sides of urethra or urine bladder, and the adjustment device decreases the distance between the elements to squeeze the urethra or urine bladder between the elements, thereby restricting the urine passageway in the urethra or urine bladder. It is also possible to use only one element and squeeze the urethra or urine bladder against human bone or tissue. The elements above may as well as all the restriction members mentioned in this application be everything from rigid to soft.

In accordance with a tenth adjustment principle, the restriction device. bends or rotates a portion of the urethra or urine bladder to restrict the urine passageway in the same. For example, the restriction device may comprise at least two bending members, such as cylindrical or hour-glass shaped rollers, positioned on opposite or different sides of the urethra or urine bladder and displaced relative to each other along the urethra or urine bladder, and the adjustment device may move the bending members against the urethra or urine bladder to bend the latter to restrict the urine passageway in the urethra or urine bladder. The restriction device may also rotate a portion of the urethra or urine bladder. The bending or rotating members may take any shape or form and be either hydraulic or non-inflatable.

Alternatively, the two bending members one placed more distal than the other may be rotated in opposite directions relative to each other. With interconnecting material for example flexible bands between the bending members a restriction will occur between the bending members when they are rotated. The restriction device may in all applicable embodiments take any shape or form and be either hydraulic or non-inflatable.

In all of the above-described embodiments the adjustment device is conveniently operated by any suitable motor, preferably an electric motor, which may be fixed directly to or be placed in association with the restriction device, or alternatively be located remote from the restriction device, advantageously in the abdomen or pelvic region or subcutaneously or in the retroperitoneum of the patient. In the latter alternative the motor is advantageously connected to the adjustment device by a flexible power transmission conduit to permit a suitable positioning of the motor in the abdomen of the patient. The motor may be manually activatable, for example by an implanted switch.

In some of the above described embodiments of the invention, however, the adjustment device may conveniently be operable by a hydraulic operation device, which preferably is manually activatable. The hydraulic operation device may advantageously include hydraulic servo means to facilitate manual activation. As an alternative, the hydraulic device may be powered by an electric motor, which may be manually activatable or controlled by remote control means. The components of such a hydraulic operation device may be placed in association with the restriction device and/or be located at a suitable place in the abdomen or be subcutaneously implanted.

More specifically, a reservoir may be provided containing a predetermined amount of fluid for supplying the hydraulic operation device with fluid. The reservoir defines a chamber for the predetermined amount of fluid and the hydraulic operation device changes the size of the chamber. The hydraulic operation device may comprise first and second wall portions of the reservoir, which are displaceable relative to each other to change the size of the chamber of the reservoir. The first and second wall portions of the reservoir may be designed to be displaceable relative to each other by manual manipulation thereof, preferably to—permit manual pushing, pulling or rotation of any of the wall portions in one direction. Alternatively, the wall portions may be displaceable relative to each other by magnetic means (such as a permanent magnet and magnetic material reed switch, or other known or conventional magnetic devices), hydraulic means or electrical control means such as an electric motor.

The magnetic means, hydraulic means, or electrical control means may all be activated by manual manipulation, preferably using a subcutaneously located manually manipulatable device. This control may be indirect, for example via a switch.

The hydraulic operation device may operate the adjustment device with fluid from the reservoir in response to a predetermined first displacement of the first wall portion of the reservoir relative to the second wall portion of the reservoir, to adjust the restriction device to release the urine passageway, and to operate the adjustment device with fluid from the reservoir in response to a predetermined second displacement of the first wall portion of the reservoir relative to the second wall portion of the reservoir, to adjust the restriction-device to restrict the urine passageway. In this embodiment, no pump is used, only the volume of the reservoir is varied. This is of great advantage compared to the solution described below when a pump is used to pump fluid between the reservoir and the adjustment device because there is no need for a non-return valve and it is still possible to have fluid going both to and from the reservoir.

As an alternative, the hydraulic operation device may comprise a pump for pumping fluid between the reservoir and the adjustment device. The pump may pump fluid both to and away from the adjustment device or hydraulic means controlling the adjustment device. A mechanical manual solution is proposed in which it is possible to pump in both directions just by pushing an activating member in one direction. Another alternative is a pump pumping in only one direction and an adjustable valve to change the direction of fluid to either increase or decrease the amount of fluid in the reservoir. This valve may be manipulated manually, mechanically, electrically, magnetically, or hydraulically. Any kind of motor could of course be used for all the different operations as well as wireless remote solutions. The pump may comprise a first activation member for activating the pump to pump fluid from the reservoir to the adjustment device and a second activation member for activating the pump to pump fluid from the adjustment device to the reservoir. The activation members may be operable by manual manipulation, preferably to permit manual pushing, pulling or rotating thereof in one direction. Suitably, at least one of the activation members is adapted to operate when subjected to an external pressure exceeding a predetermined magnitude.

Alternatively, at least one of the first and second activating, members may be operable by magnetic means, hydraulic means or electrical control means such as an electric motor. The magnetic means, hydraulic means, or electrical control means may all be activated by manual manipulating means preferably located subcutaneously. This activation may be indirect, for example via. a switch. Advantageously, especially when manual manipulation means are used, a servo means could be used. With servo means less force is needed for operating the adjustment device. The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible.

Alternatively, a reverse servo may be employed. The term “reverse servo” is to be understood as a mechanism that transfers a strong force acting on a moving element having a short stroke into a weak force acting on another moving element having a long stroke; i.e. the opposite function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device. The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the servo and reverse servo may be used in connection with all of the various components and solutions described in the present specification.

Preferably, the reverse servo comprises hydraulic means and a main fluid supply reservoir and eventually an additional fluid supply reservoir. Both reservoirs define a chamber containing hydraulic fluid, and the hydraulic means comprises first and second wall portions of the main fluid supply reservoir, which are displaceable relative to each other to change the volume of the chamber of the main fluid supply reservoir. The hydraulic means may control the adjustment device indirectly, e.g. via an increased amount of fluid in the main fluid supply reservoir, in response to a predetermined first displacement of the first wall portion of any of the reservoirs relative to the second wall portion of the reservoir to close the urine passageway, and to control the adjustment device in response to a second displacement of the first wall portion of any reservoir relative to the second wall portion, to indirectly adjust the restriction device to open the urine passageway. The wall portions of the reservoirs may be designed to be displaceable relative to each other by manual manipulation thereof or be displaceable relative to each other by manually pushing, pulling or rotating any of the wall portions of the reservoir in one direction. Alternatively, the wall portions of the main fluid supply reservoir may be displaceable relative to each other by magnetic means, hydraulic means or electric control means including an electric motor. The magnetic means, hydraulic means, or electrical control means may all be activated by manually manipulated means preferably located subcutaneously. This control may be indirect for example via a switch.

Even in the broadest embodiment the adjustment device may comprise a servo means. The servo means may comprise a hydraulic operation means, an electrical control means, a magnetic means, mechanical means or a manual manipulation means. The hydraulic operation means, electrical control means, mechanical means or magnetic means may be activated by manual manipulating means. Using a servo system will save the use of force when adjusting the adjustment device which may be of importance in many applications, for example when a battery cannot put out enough current although the total energy in the battery is more than enough to power the system.

In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable. All solutions may be controlled by a wireless remote control for controlling the adjustment device. The remote control may advantageously be capable of obtaining information related to the urine passageway or the pressure against the restriction device, urethra, or urine bladder, or other important physical parameters and of commanding the adjustment device “to adjust the restriction device in response to obtained information. With the wireless remote control the apparatus is conveniently controlled by the patient when he so desires, which, is of great advantage compared to the prior art procedures. With the remote control the apparatus is conveniently controlled to adjust the implanted restriction device to release the urine passageway when to urinate. The restriction device may be operable to open and close the urine passageway. The restriction device may steplessly control the cross-sectional area of the passageway. The apparatus may further comprise a pressure sensor for directly or indirectly sensing the pressure against the restriction device and the restriction device may control the cross-sectional area of the urine passageway in response to signals, from the pressure sensor. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor. The adjustment device preferably non-invasively adjusts the restriction device to change the restriction of the urine passageway.

The adjustment device and/or other energy consuming components of the apparatus may be energized with wirelessly transmitted energy from outside the patient's body or be powered by an implanted battery or accumulator. The apparatus may further comprise an implanted energy transforming device for transforming wireless energy directly or indirectly into kinetic energy for operation of the restriction device. The energy transforming device may, preferably directly, transform the wireless energy in the form of sound waves into electric energy for operation of the restriction device. Suitably the energy transforming device comprises a capacitor adapted to produce electric pulses from the transformed electric energy.

The apparatus may further comprise an energy transfer means for wireless transfer of energy from outside the patient's body to the—adjustment device and/or other energy consuming implantable components of the apparatus. The energy transfer means may be adapted to intermittently transfer the energy, preferably electric energy, in the form of a train of energy pulses for direct use in connection with the energizing of the energy consuming components of the apparatus. An implanted capacitor having a capacity less than 0.1 μF may be used for producing the train of energy pulses.

A motor may be implanted for operating the adjustment device, wherein the energy transfer means is adapted to directly power the motor with transferred energy. Alternatively, or in combination with the motor, a pump may be implanted for operating the adjustment device, wherein the energy transfer means is adapted to transfer wireless energy in the form of electromagnetic waves for direct power of the pump. Preferably, the pump is not a plunger type of pump, but may comprise a peristaltic or membrane pump.

The energy transfer means preferably transfers wireless energy in the form of electromagnetic waves. However, for safety radio waves may be excluded. Alternatively, the energy transferred by the energy transfer means may comprise an electric field or a magnetic field. Most preferred, the energy transferred by the energy transfer means comprises a signal.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter. The apparatus may further comprise an implanted energizer unit for providing energy to energy consuming implanted components of the apparatus, such as electronic circuits and/or a motor for operating the adjustment device. The apparatus may comprise an external energy transmitter for transmitting wireless energy, wherein the energizer unit is adapted to transform the wireless energy into electric energy. An implanted electric motor may operate the adjustment device and the energizer unit may be adapted to power the electric motor with the electric energy transformed from the wireless energy.

The energizer unit may comprise a battery and a switch operable by the wireless energy transmitted by the external transmitter, for connecting the battery to the implanted energy consuming components of the apparatus in an “on” mode when the switch is powered by the wireless energy and to keep the battery disconnected from the energy consuming components, in a “standby” mode when the switch is not powered. The control unit may power such an implanted motor with energy provided by the energizer unit in response to a control signal received from the signal transmitter. Any known or conventional signal transmitter or signal receiver that is suitable for use with a human or mammal patient may be provided as the signal transmitter or signal receiver of the invention.

Generally, all of the above signals may comprise electromagnetic waves, such as infrared light, visible light, laser light, micro waves, or sound waves, such as ultrasonic waves or infrasonic waves, or any other type of wave signals. The signals may also comprise electric or magnetic fields, or pulses. All of the above-mentioned signals may comprise digital signals. The signals may be carried by a carrier wave signal, which in an alternative embodiment may be the same signal as the wireless energy signal. Preferably a digital control signal may be carried by an electromagnetic wave signal. The carrier wave or control signal may be amplitude or frequency modulated. The motor may be any type of motor, such as a pneumatic, hydraulic or electric motor and the energizer unit may power the motor with pressurized gas or liquid, or electric energy, depending on the type of motor. Where the motor is an electric motor, it. ay power pneumatic or hydraulic equipment. The energizer unit may comprise a power supply and the control unit may power the motor with energy from the power supply. Preferably, the power supply is an electric power supply, such as a battery, and the motor is an electric motor. In this case, the battery also continuously powers at least part of the circuitry of the signal receiver in a standby mode between the adjustments, in order to keep the signal receiver prepared for receiving signals transmitted from the signal transmitter.

The energizer unit may transform energy from the control signal, as the control signal is transmitted to the signal receiver, into electric energy for powering the implanted electronic components. For example, the energizer unit may transform the energy from the control signal into a direct or alternating current.

In case there is an implanted electric motor for operating the adjustment device the energizer unit may also power the motor with the transformed energy. Advantageously, the control unit directly powers the electric motor with electric energy, as the energizer unit transforms the signal energy into the electric energy. This embodiment is particularly simple and does not require any recurrent invasive measures for exchanging empty power supplies, such as batteries, that is required in the first embodiment described above. The motor may also be directly powered with wirelessly transmitted electromagnetic or magnetic energy in the form of signals, as the energy is transmitted. All the various functions of the motor and associated components described in the present specification may be used where applicable.

For adjustment devices of the type that requires more, but still relatively low, power for its operation, the energizer unit may comprise a rechargeable electric power supply for storing the electric energy obtained and the control unit may power the electric motor with energy from the rechargeable electric power supply in response to a control signal received from the signal transmitter. In this case, the rechargeable power supply can be charged over a relatively long time (e.g. a few seconds up to a half-hour) without powering the electric motor.

The electric power supply suitably comprises an inexpensive simple capacitor. In this case, the electric motor may be a stepping motor. In all embodiments the motor may preferably be able to perform a reversing function.

The signal transmitter may transmit an electromagnetic signal and the energizer unit may draw radiant energy from the electromagnetic wave signal, as the latter is transmitted to the signal receiver, and transform the radiant energy into electric energy.

Alternatively, the energizer unit may comprise a—battery or accumulator, an electrically operable switch adapted to connect the battery to the signal receiver in an on mode when the switch is powered and to keep the battery disconnected from the signal receiver in a standby mode when the switch is not powered, and a rechargeable electric power supply for powering the switch. The control unit may power the electric motor with energy from the battery in response to a control signal received from the signal transmitter, when the switch is in its on mode. Advantageously, the energizer unit may transfer wave energy from the control signal, as the latter is transmitted to the signal receiver, into a current for charging the rechargeable electric power supply, which suitably is a capacitor. Energy from the power supply is then used to change the switch from off (standby mode) to on. This embodiment is suited for adjustment devices of the type that require relatively high power for their operation and has the advantage that the electronic circuitry of the signal receiver does not have to be powered by the battery between adjustments. As a result, the lifetime of the battery can be significantly prolonged. The switch may be switched with magnetic, manual or electric energy.

As an example, the signal transmitter may transmit an electromagnetic wave signal and the energizer unit may draw radiant energy from the electromagnetic wave signal, as the latter is transmitted. to the signal receiver, and may transform the radiant energy into the current. The energizer unit suitably comprises a coil of the signal receiver for inducing an alternating current as the electromagnetic wave signal is transmitted through the coil and a rectifier for rectifying the alternating current. The rectified current is used for charging the rechargeable power source.

Alternatively, the signal transmitter and receiver may solely, be used for a control signal and a further pair of signal transmitter and receiver may be provided for transferring signal energy to implanted components. By such a double system of signal transmitters and receivers the advantage is obtained that the two systems can be designed optimally for their respective purposes, namely to transmit a control signal and to transfer energy from an energy signal. Accordingly, the apparatus may further comprise an external energy transmitter for transmitting wireless energy, wherein the energizer unit comprises a battery and an operable switch for connecting the battery to the signal receiver in an “on” mode when the switch is powered and for keeping the battery disconnected from the signal receiver in a “standby” mode when the switch is not powered, and the external, energy transmitter powers the switch. Suitably, the energy transmitter may directly power the switch with the wireless energy to switch into the “on” mode.

As should be realized by a skilled person, in many of the above-described embodiments the adjustment device may be operated by control means or manual manipulation means implanted under the skin of the patient, such as a pump, an electrical switch or a mechanical movement transferring means. In the manual embodiment it is not necessary to use a motor for operating the adjustment device.

In embodiments including hydraulic transmission means, an injection port connected to the hydraulic means may be provided for enabling, normally single, once-and-for-all, calibration of the amount of fluid in the hydraulic system.

In all embodiments a motor may be operatively connected to the adjustment device. A reversing device may be implanted in the patient for reversing the motor.

The adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces. The restriction device is operable to open and close the urine passageway steplessly and preferably controlled by a remote control. Preferably, a pressure sensor is used for directly or indirectly sensing the pressure against the restriction device or urethra to prevent any necrosis of the human tissue to occur. The restriction device may preferably be controlled in response to signals from the pressure sensor. The motor which preferably is used to adjust the restriction device must then be capable of performing a reversible function that is to say, be capable of reversing the driving direction of the motor.

Preferably the adjustment device is directly energized with wirelessly transmitted energy from outside the patient's body. The implanted energy transforming device transforms wireless energy directly or indirectly into kinetic energy for operation of the restriction device. In accordance with another embodiment it would also be possible to use an implanted accumulator or battery and control this implanted energy source from outside the patient's body to supply energy to the adjustment device or other energy consuming implanted-parts of the apparatus.

It is possible to use two or more restricting devices for engaging the urethra or urine bladder.

All the above-described various components, such as the motor, pump and capacitor, may be combined in the different embodiments where applicable. Also the various functions described in connection with the above embodiments may be used in different applications, where applicable.

All the various ways of transferring energy and controlling the energy presented in the present specification may be practiced by using all of the various components and solutions described.

The invention also provides a method for treating urinary incontinence of a patient comprising surgically implanting in the body of a patient suffering from urinary incontinence an adjustable, preferably non-inflatable restriction device which directly engages the urethra or urine bladder to restrict the urine passageway, and when desired, mechanically adjusting the restriction device to temporarily open the urine restriction device.

The invention further provides a method for treating urinary incontinence, comprising the steps of placing at least two laparascopical trocars in the body of a patient suffering from urinary incontinence,—inserting a dissecting tool—through the trocars and dissecting in area of the urethra or urine bladder in the abdominal or pelvic or retroperitoneal surroundings, placing at least one adjustable restriction device in the dissected area engaging the urethra and adjusting the restriction device to normally, restrict the urine passageway and to release the urine passageway when the patient wants to urinate. A mechanical restriction device may be used when practicing this method, preferably also in a non-manual manner i.e. without touching subcutaneously implanted components of the apparatus. A powered adjustment device working in a non-magnetic manner may also be used.

The method may further comprise implanting a source of energy in the patient and providing a control device for controlling the source of energy from outside the patient's body to supply energy to the restriction device.

### AG: Urinary Incontinence Treatment Apparatus 2

The present aspect relates to a urinary incontinence treatment apparatus, comprising a restriction device implantable in a patient suffering from urinary incontinence for engaging the urethra or urine bladder to form a restricted urine passageway in the urethra or urine bladder, wherein the restriction device is operable to change the restriction of the urine passageway.

The object of the present aspect is to provide a new convenient urinary incontinence treatment apparatus, the performance of which may be affected by the patient at any time after operation, in particular when various needs arise over the course of a day, so that the patient substantially always is satisfied or comfortable. This object is achieved by a urinary incontinence treatment apparatus of the kind stated initially, which is characterized in that a source of energy is provided, and a control device operable from outside the patient's body is provided for controlling the source of energy to release energy for use in connection with the operation of the restriction device, when the restriction device is implanted.

As a result, the advantage is achieved that the restriction device can be non-invasively operated, when the restriction device has to be adjusted. Furthermore, the apparatus provides a simple and effective control of the energy supplied to implanted components of the apparatus which ensures an extended and reliable functionality of the apparatus, possibly for the rest of the patient's life and at least many years.

The control device may also control the restriction device. The control device may comprise an internal control unit, preferably including a microprocessor, implantable in the patient for controlling the restriction device. The control device may further comprise an external control unit outside the patient's body, wherein the internal control unit is programmable by the external control unit, for example for controlling the restriction device over time. Alternatively, the internal control unit may control the restriction device over time in accordance with an activity schedule program, which may be adapted to the patient's needs.

A great advantage is that the patient is enabled to adjust the restriction of the urine passageway by using the control device whenever he likes during the day.

Conveniently, the external control unit may load the internal control unit with data in accordance with a loading mode only authorized for a doctor. For specialized controls of the restriction device, the external control unit may control the internal control unit in accordance with a doctor mode only authorized for the doctor. For simple controls of the restriction device, the external control unit may control the internal control unit in accordance with a patient mode permitted for the patient. Thus, by using the external control unit in accordance with different modes it is possible to have certain functions of the restriction device controlled by the patient and other more advanced functions controlled by the doctor, which enables a flexible post-operation treatment of the patient.

The control device may be adapted to control the source of energy to release energy, for instance to intermittently release energy in the form of a train of energy pulses, for direct use in connection with the operation of the restriction device. In accordance with a suitable embodiment the control device controls the source of energy to release electric energy, and the apparatus further comprises an implantable capacitor for producing the train of energy pulses from the released energy. In this case the term “direct” is used to mean, on one hand, that the released energy is used while it is being released by the control device, on the other hand, that the released energy may be somewhat delayed, in the order of seconds, by for instance an energy stabilizer before being used in connection with the operation of the restriction device. The restriction device may be operable in non-manual, a non-magnetic or non-mechanical manner by use of the released energy.

In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

Generally, the apparatus further comprises an operation device implantable in the patient for operating the restriction device, wherein the control device controls the operation device to operate the restriction device. The control device may directly power the operation device with energy released from the source of energy and/or power other implantable energy consuming components of the apparatus. In this case the term “directly” is used to mean, on one hand, that the operation device is powered by released energy while the latter is being released by the control device, on the other hand, that the released energy may be somewhat delayed, in the order of seconds, by for instance an energy stabilizer before powering the operation device. The advantage of directly using energy as it is released is that the apparatus can be of a very simple design and the few components involved makes the apparatus reliable. The control device may release magnetic, electromagnetic, kinetic, sonic or thermal energy, or non-magnetic, non-sonic, non-thermal, non-electromagnetic or non-kinetic energy.

However, preferably the operation device comprises an electrical operation device. Typically the apparatus comprises an adjustment device for adjusting the restriction device to change the restriction of the urine passageway. The adjustment device may be adapted to mechanically adjust the restriction device. Alternatively, the adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces. The restriction device may be non-inflatable, i.e. with no hydraulic fluid involved for the adjustments of the restriction device. This eliminates problems with fluid leaking from the restriction device.

The operation device may comprise hydraulic means and at least one valve for controlling a fluid flow in the hydraulic means. The control device may suitably comprise a wireless remote control for controlling the valve. The restriction device may comprise hydraulic means and the operation device may comprise a reservoir forming a fluid chamber with a variable volume connected to the hydraulic means. The operation device may distribute fluid from the chamber to the hydraulic means by reduction of the volume of the chamber and withdraw fluid from the hydraulic means to the chamber by expansion of the volume of the chamber.

In accordance with a first main aspect of the invention, the source of energy is external to the patient's body and the control device controls the source of energy to release wireless energy. The external source of energy may be of any conceivable kind, such as a nuclear source of energy or a chemical source of energy.

An energy storage device, preferably an electric accumulator, may be implantable in the patient for storing the wireless energy released from the external source of energy. The electric accumulator may comprise at least one capacitor or at least one rechargeable battery, or a combination of at least one capacitor and at least one rechargeable battery. Alternatively, a battery may be implantable in the patient for supplying electric energy to implanted electric energy consuming components of the apparatus, in addition to the supply of wireless energy. Where the control device comprises an implantable control unit the electronic circuit thereof and the restriction device may be directly powered by transformed wireless energy, or energy from either the implantable energy storage device or battery.

In accordance with a second main aspect of the invention, the wireless energy is directly used for operation of the restriction device, i.e. the restriction device is operated as the wireless energy is released from the external source of energy by the control device. In this case the term “directly” is used to mean, on one hand, that the restriction device is promptly operated by using the released energy without first storing the latter, on the other hand, that the released energy may be somewhat delayed, in the order of seconds, by for instance an energy stabilizer before being used for the operation of the restriction device. As a result, a very simple control of the restriction device is achieved and there are only a few implanted components of the apparatus. For example, there is no implanted source of energy, such as a battery, nor any implanted complicated signal control system. This gives the advantage that the apparatus will be extremely reliable.

Generally, the control device controls and directly or indirectly powers the operation device with wireless energy released from the source of energy and/or powers other implanted energy consuming components of the apparatus.

In a first particular embodiment in accordance with the first and second main aspects of the invention, the operation device comprises a motor, preferably an electric motor which may have electrically conductive parts made of plastics. The motor may include a rotary motor, wherein the control device is adapted to control the rotary motor to rotate a desired number of revolutions. Alternatively, the motor may include a linear motor, or a hydraulic or pneumatic fluid motor, wherein the control device is adapted to control the fluid flow through the fluid motor. Motors currently available on the market are getting smaller and smaller. Furthermore, there is a great variety of control methods and miniaturized control equipment available. For example, a number of revolutions of a rotary motor may be analyzed by a Hall-element just a few mm in size. In a second particular embodiment in accordance with the first and second main aspects of the invention, the control device is adapted to shift polarity of the released energy to reverse the operation device. The operation device may suitably comprise an electric motor and the released energy may comprise electric energy.

In a third particular embodiment in accordance with the first and second main aspects of the invention, the restriction device is operable to perform a reversible function and there is a reversing device implantable in the patient for reversing the function performed by the restriction device. Such a reversing function preferably involves enlarging and restricting the urine passageway by the restriction device, suitably in a stepless manner. In this connection, the control device suitably controls the reversing device, which may include a switch, to reverse the function performed by the restriction device. The reversing device may comprise hydraulic means including a valve for shifting the flow direction of a fluid in the hydraulic means. Alternatively, the reversing device may comprise a mechanical reversing device, such as a switch or a gearbox.

Where the reversing device comprises a switch the control device suitably controls the operation of the switch by shifting polarity of released energy supplied to the switch. The switch may comprise an electric switch and the source of energy may supply electric energy for the operation of the switch. The switch mentioned above may comprise an electronic switch or, where applicable, a mechanical switch. In accordance with the third particular embodiment, the operation device preferably comprises a motor, wherein the reversing device reverses the motor.

In a fourth particular embodiment in accordance with the first and second main aspects of the invention, the restriction device comprises hydraulic means, for example including an expansible/contractible cavity for fluid. Preferably, the operation device is adapted to conduct hydraulic fluid in the hydraulic means, and comprises a motor, a valveless fluid conduit connected to the hydraulic means of the restriction device, and a reservoir for fluid, wherein the reservoir forms part of the conduit. The operation device suitably comprises a pump operated by the motor. All of the hydraulic components involved are preferably devoid of any non-return valve. This is of great advantage, because with valves involved there is always a risk of malfunction due to improperly working valves, especially when long time periods passes between valve operations. The reservoir may form a fluid chamber with a variable volume, and the pump may distribute fluid from the chamber to the hydraulic means of the restriction device by reduction of the volume of the chamber and withdraw fluid from the hydraulic means to the chamber by expansion of the volume of the chamber. In accordance with a third main aspect of the invention, the source of energy is implantable in the patient. Thus, when the source of energy is implanted in a patient the control device controls it from outside the patient's body to release energy. This solution is advantageous for embodiments of the apparatus that have a relatively high consumption of energy, which cannot be satisfied by direct supply of wireless energy.

The implantable source of energy may comprise an accumulator, preferably an electric source of energy, such as a battery having a lifetime of at least 10 years. In accordance with a fourth main aspect of the invention, the apparatus comprises a switch implanted in the patient for directly or indirectly switching the operation of the restriction device and an internal source of energy, such as a battery, implanted in the patient for supplying energy for the operation of the restriction device, wherein the switch directly or indirectly affects the supply of energy from the internal source of energy. This solution is advantageous for embodiments of the apparatus that have a relatively high energy consumption which cannot be met by direct supply of wireless energy. In a first particular embodiment in accordance with the fourth main aspect of the invention, the switch switches between an off mode, in which the internal source of energy is not in use, and an on mode, in which the internal source of energy supplies energy for the operation of the restriction device. In this case, the switch is conveniently operated by the wireless energy released from the external source of energy to switch between the on and off modes. The control device, preferably comprising a wireless remote control, may control the external source of energy to release the wireless energy. The advantage of this embodiment is that the lifetime of the implanted source of energy, such as a battery, can be significantly prolonged, since the implanted source of energy does not supply energy when the switch is in its off mode.

In a second particular embodiment in accordance with the fourth main aspect of the invention, the control device comprises a wireless remote control for controlling the internal source of energy. In this case, the switch is operable by the wireless energy from the external source of energy to switch between an off mode, in which the internal source of energy and remote control are not in use, and a standby mode, in which the remote control is permitted to control the internal source of energy to supply energy for the operation of the restriction device.

In a third particular embodiment in accordance with the fourth main aspect of the invention, the apparatus further comprises an energy transforming device implanted in the patient for transforming the wireless energy into storable energy, wherein the internal source of energy is capable of storing the storable energy. The internal source of energy preferably comprises an electric accumulator, at least one capacitor or at least one rechargeable battery, or a combination of at least one capacitor and at least one rechargeable battery. In this case, the switch switches from an off mode, in which the internal source of energy is not in use, to an on mode, in which the internal source of energy supplies energy for the operation of the restriction device.

The control device, preferably comprising a wireless remote control, may control the switch to switch between the on and off modes. Alternatively, in this third particular embodiment an energy storage device may be implanted in the patient for storing the storable energy instead of the internal source of energy, wherein the switch is operable by energy from the implanted energy storage device to switch between an off mode, in which the internal source of energy is not in use, and an on mode, in which the internal source of energy supplies energy for the operation of the restriction device. In this case, the control device (the wireless remote control) controls the energy storage device to operate the switch. The internal source of energy preferably comprises an electric source of energy, such as an accumulator or a battery having a lifetime of at least 10 years. However, other kinds of sources are also conceivable, such as a nuclear source of energy or a chemical source of energy. The above first, second, third and fourth particular embodiments described in connection with the first and second main aspects are also applicable in accordance with the third main aspect of the invention, i.e. where the source of energy is implantable, and in accordance with the fourth main aspect of the invention, i.e. where the apparatus comprises an implantable switch.

All of the above embodiments may be combined with at least one implantable sensor for sensing at least one physical parameter of the patient, wherein the control device may control the restriction device in response to signals from the sensor. For example, the sensor may comprise a pressure sensor for directly or indirectly sensing the pressure in the urethra or urine bladder. The expression “indirectly sensing the pressure in the urethra or urine bladder” should be understood to encompass the cases where the sensor senses the pressure against the restriction device or human tissue of the patient. Where the control device comprises an internal control unit to be implanted in the patient, the internal control unit may suitably directly control the restriction device in response to signals from the sensor. In response to signals from the sensor, for example pressure, the patient's position or any other important physical parameter, the internal control unit may send information thereon to outside the patient's body. The control unit may also automatically control the restriction device in response to signals from the sensor. For example, the control unit may control the restriction device to firmly close the urine passageway in response to the sensor sensing that the patient is lying, or enlarge the urine passageway in response to the sensor sensing an abnormally high pressure against the restriction device.

Where the control device comprises an external control unit outside the patient's body, the external control unit may, suitably directly, control the restriction device in response to signals from the sensor. The external control unit may store information on the physical parameter sensed by the sensor and may be manually operated to control the restriction device based on the stored information. In addition, there may be at least one implantable sender for sending information on the physical parameter sensed by the sensor.

An external data communicator may be provided outside the patient's body and an internal data communicator to be implanted in the patient may be provided for communicating with the external data communicator. The internal data communicator may feed data related to the patient, or related to the restriction device, back to the external data communicator. Alternatively or in combination, the external data communicator may feed data to the internal data communicator. The internal data communicator may suitably feed data related to at least one physical signal of the patient.

Generally, the apparatus may comprise a switch implantable in the patient for directly or indirectly switching the energy released from the source of energy. For example, the restriction device may be operable to open and close the urine passageway or may steplessly control the restriction of the urine passageway. A pressure sensor may be provided for directly or indirectly sensing the pressure in the urethra or urine bladder. The control device may control the restriction device in response to signals from the pressure sensor. The apparatus may comprise an implantable energy transforming device, wherein the control device releases electric energy and the energy transforming device transforms the electric energy into kinetic energy for, preferably direct, operation of the restriction device. Suitably, an implantable stabilizer, such as a capacitor or a rechargeable accumulator, or the like, may be provided for stabilizing the electric energy released by the control device. In addition, the control device may control the source of energy to release energy for a determined time period or in a determined number of energy pulses. Finally, the restriction device may be non-inflatable.

All of the above embodiments are preferably remote controlled. Thus, the control device advantageously comprises a wireless remote control transmitting at least one wireless control signal for controlling the restriction device. With such a remote control it will be possible to adapt the function of the apparatus to the patient's need in a daily basis, which is beneficial with respect to the treatment of the patient. The wireless remote control may be capable of obtaining information on the condition of the restriction device and of controlling the restriction device in response to the information. Also, the remote control may be capable of sending information related to the restriction device from inside the patient's body to the outside thereof.

In a particular embodiment of the invention, the wireless remote control comprises at least one external signal transmitter or transceiver and at least one internal signal receiver or transceiver implantable in the patient. In another particular embodiment of the invention, the wireless remote control comprises at least one external signal receiver or transceiver and at least one internal signal transmitter or transceiver implantable in the patient. The remote control may transmit a carrier signal for carrying the control signal, wherein the carrier signal is frequency, amplitude or frequency and amplitude modulated and is digital, analog or digital and analog. Also the control signal used with the carrier signal may be frequency, amplitude or frequency and amplitude modulated.

The control signal may comprise a wave signal, for example, a sound wave signal, such as an ultrasound wave signal, an electromagnetic wave signal, such as an infrared light signal, a visible light signal, an ultra violet light signal, a laser signal, a micro wave signal, a radio wave signal, an x-ray radiation signal, or a gamma radiation signal. Where applicable, two or more of the above signals may be combined.

The control signal may be digital or analog, and may comprise an electric or magnetic field. Suitably, the wireless remote control may transmit an electromagnetic carrier wave signal for carrying the digital or analog control signal. For example, use of an analog carrier wave signal carrying a digital control signal would give safe communication. The control signal may be transmitted in pulses by the wireless remote control.

In all of the above solutions, the control device advantageously releases energy from the source of energy in a non-invasive, magnetic, non-magnetic, mechanical or non-mechanical manner.

The control device may release magnetic, electromagnetic, kinetic or thermal energy, or non-magnetic, non-thermal, non-electromagnetic or non-kinetic energy. The control device may be activated in a manual or non-manual manner to control the source of energy to release energy. The above-presented embodiments may be modified in accordance with the following suggestions. The released energy may comprise electric energy and an implantable capacitor having a capacity less than 0.1 μF may be provided for producing the above-mentioned train of energy pulses.

An implantable motor or pump may be provided for operating the restriction device, wherein the control device is adapted to control the source of energy to directly power the motor or pump with the released energy. Specifically, the control device may be adapted to release wireless energy in the form of a magnetic field or electromagnetic waves (excluding radio waves) for direct power of the motor or pump, as the wireless energy is being released. Where a pump is used it preferably is not a plunger type of pump.

Generally, the wireless energy comprises a signal.

The apparatus may further comprise implantable energy transforming device for transforming wireless energy directly or indirectly into energy different than the wireless energy, for operation of the restriction device. For example, the motor or pump may be powered by the transformed energy.

The energy transforming device may transform the wireless energy in the form of sound waves, preferably directly, into electric energy for operation of the restriction device. The energy transforming device may comprise a capacitor adapted to produce electric pulses from the transformed electric energy.

The motor mentioned in the present specification may also be directly powered with wirelessly transmitted electromagnetic or magnetic energy in the form of signals, as the energy is transmitted. Furthermore, all the various functions of the motor and associated components described in the present specification may be used where applicable.

Generally, the restriction device advantageously is embedded in a soft or gel-like material, such as a silicone material having hardness less than 20 Shore.

Of course, the restriction device preferably is adjustable in a non-manual manner.

All the above described various components, such as the motor, pump and capacitor, may be combined in the different embodiments where applicable. Also the various functions described in connection with the above embodiments may be used in different applications, where applicable.

All the various, ways of transferring energy and controlling the energy presented in the present specification may be practiced by using all of the various components and solutions described.

The present invention also provides methods for treating urinary incontinent patients.

Accordingly, in accordance with a first alternative method, there is provided a method of treating a patient suffering from urinary incontinence, comprising the steps of implanting an operable restriction device in the patient, so that the restriction device engages the urethra or urine bladder to form a restricted urine passageway in the urethra or urine bladder, providing a source of energy for energizing the restriction device, and controlling the source of energy to release energy for use in connection with the operation of the restriction device. The method may further comprise using energy released from the source of energy to operate the restriction device to open and close, respectively, the urine passageway.

In accordance with a second alternative method, there is provided a method of treating a patient suffering from urinary incontinence, comprising the steps of placing at least two laparascopical trocars in the patient's body, inserting a dissecting tool through the trocars and dissecting an area of the urethra or urine bladder, placing an operable restriction device in the dissected area, so that the restriction device engages the urethra or urine bladder to form a restricted urine passageway in the urethra or urine bladder, implanting a source of energy in the patient, and controlling the implanted source of energy from outside the patient's body to release energy for use in connection with the operation of the restriction device. In accordance with a third alternative method, there is provided a method of treating a patient suffering from urinary incontinence, comprising: (a) Surgically implanting in the patient an operable restriction device engaging the patient's urethra or urine bladder to form a restricted urine passageway in the urethra or urine bladder, (b) Providing a source of energy external to the patient's body. (c) Controlling the external source of energy from outside the patient's body to release wireless energy. And (d) using the released wireless energy in connection with the operation of the restriction device. The method may further comprise (e) implanting in the human or animal an operation device which can adjust the restricted urine passageway in response to supplied energy, and (f) using the released wireless energy to activate the implanted operation device so as (i) to enlarge the restricted urine passageway to allow urine to readily pass therethrough but normally restrict the urine passageway. In the method (f) may be practiced at least once a day, normally several times (e.g. 2-10) a day.

In accordance with a fourth alternative method, there is provided a method of treating a patient suffering from urinary incontinence, comprising the steps of placing at least two laparascopical trocars in the patient's body, inserting a dissecting tool through the trocars and dissecting an area of the urethra or urine bladder, placing an operable restriction device in the dissected area, so that the restriction device engages the urethra or urine bladder to form a restricted urine passageway in the urethra or urine bladder, providing an external source of energy outside the patient's body, controlling the external source of energy from outside the patient's body to release wireless energy, and using the released wireless energy in connection with the operation of the restriction device.

In accordance with a fifth alternative method, there is provided a method of treating a patient suffering from urinary incontinence, comprising the steps of placing at least two laparascopical trocars in the patient's body, inserting a dissecting tool through the trocars and dissecting an area of the urethra or urine bladder, implanting an operable restriction device in the dissected area, so that the restriction device engages the urethra or urine bladder to form a restricted urine passageway in the urethra or urine bladder, implanting an energy transforming device, providing an external source of energy, controlling the external source of energy to release wireless energy, and transforming the wireless energy by the energy transforming device into energy different than the wireless energy for use in connection with the operation of the restriction device. This method may further comprise implanting a stabilizer in the patient for stabilizing the energy transformed by the energy transforming device.

### AH: An Apparatus for Temporary Male Constriction

In general terms, the present aspect relates to a male contraception apparatus for obtaining temporary sterility of a male mammalian individual. The apparatus comprises an implantable restriction device adapted to restrict vas deference in the region downstream of the ampulla during a controlled period, said device thereby being capable of preventing sperms to reach the urethra. The apparatus further comprises a control device for controlling the operation of the restriction device. The restriction device may comprise a hydraulic or mechanical constriction device as well as a stimulation device for, independently or together in any combination, temporarily restrict vas deferens. In this context restriction of vas deference means that this lumen is occluded in a manner to prevent sperms to reach the urethra by operating on vas deference from the outside. The terms “vas deference” may include one vas deference or both vasa deferentia. When explaining the controlled restriction of vas deference according to the invention also other terms like “lumen” or “tissue portion” or “body organ” are used, but such terms shall be regarded as functional synonyms. The term “ampulla” refers to the enlargement of vas deference close to where it meets the seminal vesicle.

The term “downstream the ampulla” refers to a location after the ampulla in the direction of the urethra and the seminal vesicle. Consequently, “upstream the ampulla” would refer to a location on the vas deference before the ampulla in the direction towards the testicle. That the restriction device is adapted to restrict vas deference in this specific region preferably means that it is designed to be accommodated in this region of the body.

By accomplishing a restriction of vas deference downstream of the ampulla, the presently invented apparatus admits a reliable temporary sterilization by shielding of sperms compartmentalized in the ampulla, whereby the apparatus immediately may exert its contraceptive effect. Even if the principal utility of the apparatus is to prevent sperm transport for a controlled, temporary period, for example during sexual intercourse by activating the restriction device, certain embodiments also admits support of transporting sperms through vas deference to assist individuals having impairments in this transportation system. Advantageously, the present invention admits a very limited time period the restriction needs to be restricted. Existing treatments need to be closed at least 5 days before it is safe, the life time of a sperm, thus risking damaging vas deferens. Furthermore, the apparatus prevents from that sperms accumulate in the vas deferens due to long term restriction which may lead to undesired complications.

The restriction device is very carefully be adapted to the specific position downstream the ampulla. The amount of space is limited and the requirements are specific to allow placement in this region between the prostate and the ampulla of vas deferens. Because the ampulla comprises a reservoir of sperm the restriction is performed in the downstream region to prevent any possibility to sperm reaching the urethra during intercourse. The restriction device according to invention can also when relevant be adapted to accomplish restriction of closely located outlet duct of seminal vesicles. A simultaneous restriction of this outlet duct should be regarded as one of the effects obtainable with the present invention.

According to one embodiment, restriction device comprises a constriction device for constricting at least one portion of a tissue wall of vas deference downstream the ampulla to stop the flow in the vas deference. For this purpose, the apparatus comprises an adjustable restriction device and an operation device for mechanically or hydraulically operating the adjustable restriction device to change the restriction of a wall portion of vas deference. The operation device preferably operates the restriction device in a non-magnetic and/or non-manual manner. Preferably, the operation device comprises an electrically powered operation device, such as motor or a servo system. The term “servo system” means that the system includes a mechanism that transfers a weak force acting on a moving element having a long stroke into a strong force acting on another moving element having a short stroke. According to one alternative the restriction device comprises a constriction device comprising at least two elongated clamping elements extending along the organ in the direction of flow in the patient's vas deference on different sides of the organ, and the operation device operates the clamping elements to clamp the wall portion between the clamping elements to constrict the wall portion. The operation device can alternatively comprise hydraulic means for hydraulically adjusting the restriction device and a reverse servo operatively connected to the hydraulic means. The term “reverse servo” is to be understood as a mechanism that transfers a strong force acting on a moving element having a short stroke into a weak force acting on a moving element with a long stroke. Preferably, the reverse servo comprises at least two implantable reservoirs comprising hydraulic fluid, and preferably a first reservoir is a subcutaneously implantable regulation reservoir. The reverse servo further comprises a second implantable servo reservoir which preferably is in fluid connection with the regulation reservoir. In one embodiment the second servo reservoir directly controls the expansion/contraction of the constriction device. In another embodiment, the servo reservoir indirectly controls the expansion/contraction of the constriction device, wherein the reverse servo further comprising a third reservoir adapted to be implanted in the abdomen or retroperitoneum or pelvic region and being operatively connected to the second servo reservoir for displacing hydraulic fluid of said third reservoir to operate the constriction device. Suitably, the third reservoir has a larger volume than the first reservoir. The third reservoir can be in fluid connection with the constriction device and the servo reservoir can control the third reservoir with a mechanical interconnection Alternatively, the restriction device comprises a non-inflatable mechanical constriction device and the operation device comprises hydraulic means that hydraulically adjusts the mechanical constriction device.

According to another embodiment of the invention, the restriction device comprises a stimulation device for stimulating a wall portion of the tissue wall of vas deference in the region downstream the ampulla to contract said wall portion to influence the flow in vas deference. A control device controls the stimulation device to stimulate the wall portion and the control device is preferably operable from outside the patient's body to control an implantable source of energy to release energy for use in connection with the stimulation. The stimulation device is adapted to stimulate different areas of the wall portion and the control device controls the stimulation device to intermittently and individually stimulate the areas of the wall portion. This intermittent and individual stimulation of different areas of the wall portion of the organ allows tissue of the wall portion to maintain substantially normal blood circulation during the operation of the apparatus of the invention. Various alternatives of accomplishing restriction by stimulation and useful components are described below.

It is also an embodiment that restriction device comprises a mechanically or hydraulically operated constriction as described above and a stimulation device as described above. The control device is adapted to control the constriction device and the stimulation device to in combination restrict vas deference.

The aspect also embodies a method of stimulating the ampulla comprising employing an apparatus as described having a stimulation device for restricting vas deference downstream the ampulla. A counterflow stimulation of the ampulla is thereby obtainable.

Further the invention comprises a system including any apparatus described in previous sections. In a preferred embodiment, the system comprises at least one switch implantable in the patient for manually and non-invasively controlling the apparatus. In another preferred embodiment, the system comprises a wireless remote control for non-invasively controlling the apparatus. In a preferred embodiment, the system comprises a hydraulic operation device for operating the apparatus. In another embodiment, the system comprises comprising a motor or a pump for operating the apparatus. Other components of the system are described in more detail in the detailed part of the present description.

Stimulation When stimulating neural or muscular tissue there is a risk of injuring or deteriorating the tissue over time, if the stimulation is not properly performed. The apparatus of the present invention is designed to reduce or even eliminate that risk. Thus, in accordance with the present invention, the control device controls the stimulation device to intermittently stimulate different areas of the wall portion of the organ, such that at least two of the areas are stimulated at different points of time that is, the stimulation is shifted from one area to another area over time. In addition, the control device controls the stimulation device, such that an area of the different areas that currently is not stimulated has time to restore substantially normal blood circulation before the stimulation device stimulates the area again. Furthermore, the control device controls the stimulation device to stimulate each area during successive time periods, wherein each time period is short enough to maintain satisfactory blood circulation in the area until the lapse of the time period. This gives the advantage that the apparatus of the present invention enables continuous stimulation of the wall portion of the organ to achieve the desired flow control, while essentially maintaining over time the natural physical properties of the organ without risking injuring the organ.

Also, by physically changing the places of stimulation on the organ overtime as described above it is possible to create an advantageous changing stimulation pattern on the organ, in order to achieve a desired flow control.

The control device may control the stimulation device to stimulate one or more of the areas of the wall portion at a time, for example by sequentially stimulating the different areas.

Furthermore, the control device may control the stimulation device to cyclically propagate the stimulation of the areas along the wall portion, preferably in accordance with a determined stimulation pattern. To achieve the desired reaction of the tissue wall during the stimulation thereof, the control device may control the stimulation device to, preferably cyclically, vary the intensity of the stimulation of the wall portion.

In a preferred embodiment of the invention, the control device controls the stimulation device to intermittently stimulate the areas of the wall portion with pulses that preferably form pulse trains. At least a first area and a second area of the areas of the wall portion may be repeatedly stimulated with a first pulse train and a second pulse train, respectively, such that the first and second pulse trains over time are shifted relative to each other. For example, the first area may be stimulated with the first pulse train, while the second area is not stimulated with said second pulse train, and vice versa. Alternatively, the first and second pulse trains may be shifted relative to each other, such that the first and second pulse trains at least partially overlap each other.

The pulse trains can be configured in many different ways. Thus, the control device may control the stimulation device to vary the amplitudes of the pulses of the pulse trains, the duty cycle of the individual pulses of each pulse train, the width of each pulse of the pulse trains, the length of each pulse train, the repetition frequency of the pulses of the pulse trains, the repetition frequency of the pulse trains, the number of pulses of each pulse train, and/or the off time periods between the pulse trains. Several pulse trains of different configurations may be employed to achieve the desired effect.

In case the control device controls the stimulation device to vary the off time periods between pulse trains that stimulate the respective area of the wall portion, it is also possible to control each off time period between pulse trains to last long enough to restore substantially normal blood circulation in the area when the latter is not stimulated during the off time periods.

### Electric Stimulation

In accordance with a preferred embodiment of the invention, the stimulation device is an electrically powered stimulation device that electrically stimulates the tissue wall portion of the patient's bodily organ, preferably with electric pulses. This embodiment is particularly suited for applications in which the wall portion includes muscle fibers that react to electrical stimula. In this embodiment, the control device controls the stimulation device to stimulate the wall portion with electric pulses preferably in the form of electric pulse trains, when the wall portion is in the constricted state, to cause contraction of the wall portion. Of course, the configuration of the electric pulse trains may be similar to the above described pulse trains and the control device may control the stimulation device to electrically stimulate the different areas of the wall of the organ in the same manner as described above.

The electric stimulation device suitably comprises at least one, preferably a plurality of electrical elements, such as electrodes, for engaging and stimulating the wall portion with electric pulses. Optionally, the electrical elements may be placed in a fixed orientation relative to one another. The control device controls the electric stimulation device to electrically energize the electrical elements, one at a time, or groups of electrical elements at a time.

Preferably, the control device controls the electric stimulation device to cyclically energize each element with electric pulses. Optionally, the control device may control the stimulation device to energize the electrical elements, such that the electrical elements are energized one at a time in sequence, or such that a number or groups of the electrical elements are energized at the same time. Also, groups of electrical elements may be sequentially energized, either randomly or in accordance with a predetermined pattern.

The electrical elements may form any pattern of electrical elements. Preferably, the electrical elements form an elongate pattern of electrical elements, wherein the electrical elements are applicable on the patient's wall of the organ, such that the elongate pattern of electrical elements extends lengthwise along the wall of the organ, and the elements abut the respective areas of the wall portion. The elongate pattern of electrical elements may include one or more rows of electrical elements extending lengthwise along the wall of the organ. Each row of electrical elements may form a straight, helical or zig-zag path of electrical elements, or any form of path. The control device may control the stimulation device to successively energize the electrical elements longitudinally along the elongate pattern of electrical elements in a direction opposite to, or in the same direction as that of, the flow in the patient's lumen.

Optionally, the control device may control the stimulation device to successively energize the electrical elements from a position substantially at the center of the constricted wall portion towards both ends of the elongate pattern of electrical elements. Where the lumen of the organ is to be kept closed for a relatively long time, the control device may control the stimulation device to energize the electrical elements, such that energized electrical elements form two waves of energized electrical elements that simultaneously advance from the center of the constricted wall portion in two opposite directions towards both ends of the elongate pattern of electrical elements. Such waves of energized electrical elements can be repeated over and over again without harming the organ and without moving fluid or gas in any direction in the lumen of the organ.

The control device suitably controls the stimulation device to energize the electrical elements, such that the electrical elements currently energized form at least one group of adjacent energized electrical elements. In accordance with a first alternative, the elements in the group of energized electrical elements form one path of energized electrical elements. The path of energized electrical elements may extend at least in part around the patient's organ. In a second alternative, the elements of the group of energized electrical elements may form two paths of energized electrical elements extending on mutual sides of the patient's organ, preferably substantially transverse to the flow direction in the lumen of the organ. In a third alternative, the elements of the group of energized electrical elements may form more than two paths of energized electrical elements extending on different sides of the patient's organ, preferably substantially transverse to the flow direction in the patient's lumen.

In accordance with a preferred embodiment of the invention, the electrical elements form a plurality of groups of elements, wherein the groups form a series of groups extending along the patient's organ in the flow direction in the patient's lumen. The electrical elements of each group of electrical elements may form a path of elements extending at least in part around the patient's organ. In a first alternative, the electrical elements of each group of electrical elements may form more than two paths of elements extending on different sides of the patient's organ, preferably substantially transverse to the flow direction in the patient's lumen. The control device may control the stimulation device to energize the groups of electrical elements in the series of groups in random, or in accordance with a predetermined pattern. Alternatively, the control device may control the stimulation device to successively energize the groups of electrical elements in the series of groups in a direction opposite to, or in the same direction as that of, the flow in the patient's lumen, or in both said directions starting from a position substantially at the center of the constricted wall portion. For example, groups of energized electrical elements may form advancing waves of energized electrical elements, as described above; that is, the control device may control the stimulation device to energize the groups of electrical elements, such that energized electrical elements form two waves of energized electrical elements that simultaneously advance from the center of the constricted wall portion in two opposite directions towards both ends of the elongate pattern of electrical elements.

A structure may be provided for holding the electrical elements in a fixed orientation. Although the structure may be separate from the constriction device, it is preferable that the structure is integrated in the constriction device, which is a practical design and facilitates implantation of the constriction and stimulation devices. Where the electrical elements form an elongate pattern of electrical elements, the structure may be applicable on the patient's organ such that the elongate pattern of electrical elements extends along the organ in the same direction as that of the flow in the patient's lumen and the elements abut the respective areas of the wall portion of the organ.

### Thermal Stimulation

In another embodiment of the invention, the stimulation device thermally stimulates the wall portion of the organ. Thus, the control device may control the stimulation device to cool the wall portion, when the wall portion is constricted, to cause contraction of the wall portion. For example, the constriction device may constrict the wall portion to at least restrict the flow in the lumen, and the control device may control the stimulation device to cool the constricted wall portion to cause contraction thereof, such that the flow in the lumen is at least further restricted, or further restricted but not stopped, or stopped. Alternatively, the control device may control the stimulation device to heat the wall portion, when the wall portion is constricted and contracted, to cause expansion of the wall portion. Where the wall portion includes a blood vessel, the control device may control the stimulation device to cool the blood vessel to cause contraction thereof, or heat the blood vessel to cause expansion thereof. Where applicable, thermal stimulation may be practiced in any of the embodiments of the present invention, and the thermal stimulation may be controlled in response to various sensors, for example strain, motion or pressure sensors.

Sensor Controlled Constriction and/or Stimulation Device

As mentioned above, the apparatus may comprise at least one implantable sensor, wherein the control device controls the constriction device and/or the stimulation device in response to signals from the sensor. Generally, the sensor directly or indirectly senses at least one physical parameter of the patient, or at least one functional parameter of the apparatus, or at least one functional parameter of a medical implant in the patient.

Many different kinds of sensor for sensing physical parameters may be used. For example motion sensors for sensing organ motion, i.e. natural contractions, such as stomach or intestinal contractions, pressure sensors for sensing pressure in the organ, strain sensors for sensing strain of the organ, flow sensors for sensing fluid flow in the lumen of the organ, spectro-photometrical sensors, Ph-sensors for sensing acidity or alkalinity of the fluid in the lumen of the organ, oxygen-sensors sensors for sensing the oxygen content of the fluid in the lumen of the organ, or sensors for sensing the distribution of the stimulation on the stimulated organ. Any conceivable sensors for sensing any other kind of useful physical parameter may be used.

Many different kinds of sensors that sense functional parameters of the apparatus may also be used for the control of the constriction device and/or the stimulation device. For example sensors for sensing electric parameters of implanted electric components of the apparatus, or sensors for sensing the performance of implanted motors of the apparatus.

The sensor may comprise a pressure sensor for sensing as the physical parameter a pressure in the patient's body that relates to the pressure in the lumen of the patient's bodily organ, wherein the control device controls the constriction device and/or stimulation device to change the constriction of the patient's wall portion in response to the pressure sensor sensing a predetermined value of measured pressure.

Alternatively, or in combination with the pressure sensor, a position sensor may be provided for sensing as the physical parameter the orientation of the patient with respect to the horizontal. The position sensor may be a biocompatible version of what is shown in U.S. patents 4,942,668 and 5,900,909. For example, the control device may control the constriction device and/or stimulation device to change the constriction of the patient's wall portion in response to the position sensor sensing that the patient has assumed a substantially horizontal orientation, i.e. that the patient is lying down.

The above described sensors may be used in any of the embodiments of the invention, where applicable.

The control device may control the constriction device and/or stimulation device to change the constriction of the patient's wall portion in response to the time of day. For that purpose the control device may include a clock mechanism for controlling the constriction device and/or stimulation device to change the constriction of the patient's wall portion to increase or decrease the influence on the flow in the lumen during different time periods of the day. In case a sensor of any of the above-described types for sensing a physical or functional parameter is provided, either the clock mechanism is used for controlling the constriction device and/or stimulation device provided that the parameter sensed by the sensor does not override the clock mechanism, or the sensor is used for controlling the constriction device and/or stimulation device provided that the clock mechanism does not override the sensor.

Suitably, the control device produces an indication, such as a sound signal or displayed information, in response to signals from the sensor.

The control device may comprise an implantable internal control unit that directly controls the constriction device and/or stimulation device in response to signals from the sensor. The control device may further comprise a wireless remote control adapted to set control parameters of the internal control unit from outside the patient without mechanically penetrating the patient. At least one of the control parameters, which are settable by the wireless remote control, is the physical or functional parameter. Suitably, the internal control unit includes the above mentioned clock mechanism, wherein the wireless remote control also is adapted to set the clock mechanism.

Alternatively, the control device may comprise an external control unit outside the patient's body for controlling the constriction device and/or stimulation device in response to signals from the sensor.

### AI: Anal Incontinence Treatment Apparatus

The present aspect relates to an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway.

An object of the present aspect is to provide an anal incontinence treatment apparatus in which the risk of liquid leaking from hydraulic components of the apparatus implanted in the patient's body is substantially reduced or completely eliminated.

A further object of the aspect is to provide an anal incontinence treatment apparatus, which does not require a manual manipulation of a combined reservoir and pump mechanism implanted in the scrotum or labia majora region of the patient.

These objects are obtained by an apparatus of the kind stated initially characterized in that the adjustment device is adapted to mechanically adjust the restriction device.

The adjustment device may be non-manually operated, i.e. the adjustment device may be operated by any powered operation device, not manipulated by touching the skin of the patient. Preferably, the adjustment device is adapted to adjust the restriction device in a non-invasive manner.

The adjustment device may adjust the restriction device device in a non-thermal manner or in a non-magnetic manner, i.e. magnetic forces may not be involved when adjusting the restriction device.

The restriction device preferably is adapted to control, suitably steplessly, the cross-sectional area of the fecal passageway, i.e. to open and close the fecal passageway.

In the various embodiments hereinafter described the restriction device generally forms an at least substantially closed loop. However, the restriction device may take a variety of different shapes, such as the shape of a square, rectangle or ellipse. The substantially closed loop could for example be totally flat, i.e. thin as seen in the radial direction. The shape of restriction device may also be changed during use, by rotation or movements of the restriction device in any direction.

A physical lumen, like the colon or rectum, is often easier to restrict by contracting at least two opposite or different side walls of the lumen against each other. The expression “colon or rectum should be understood to mean the rectum extended all the way out to the anal sphincter and following’ the passage of the large intestine in the other direction.

The restriction device may comprise an adjustable cuff, a clamp or a roller for bending the colon or rectum to restrict the fecal passageway therein. Such a cuff, clamp or roller may also be utilized for squeezing the colon or rectum against human material inside the body of the patient for an example the sacral bone of the patient.

Preferably, the restriction device comprises an elongated restriction member and forming means for forming the restriction member into at least a substantially closed loop around the portion of the colon or rectum, wherein the loop defines a restriction opening, whereby the adjustment device adjusts the restriction member in the loop to change the size of the restriction opening.

The restriction device may be designed for implantation in the abdomen or retroperitoneum of the patient and preferably may engage the colon or rectum.

The adjustment device may be incorporated in the restriction device as well as controlled by hydraulic means.

In accordance with a preferred first adjustment principle, the adjustment device mechanically adjusts the longitudinal extension of the elongated restriction member in a loop form.

In a preferred embodiment utilizing the first adjustment principle, the restriction member comprises a main portion and two elongated end portions, and the adjustment device establishes longitudinal relative displacement between the end portions of the restriction member, so that the size of the restriction opening is adjusted. The forming means may comprise any suitable known or conventional device capable of practicing the desired function, such as a spring material forming the elongated restriction member into the loop, so that the restriction opening has a predetermined size, and the adjustment device may adjust the restriction member against the spring action of the spring material. In other words, the restriction member may comprise a spring clip. The spring material may be integrated in the restriction member.

Preferably, the adjustment device comprises a movement transferring member, suitably a drive wheel, in engagement with at least one of the end portions of the restriction member and operable to displace the one end portion relative to the other end portion of the restriction member. The drive wheel may advantageously be in engagement with both of the end portions of the restriction member and be operable to displace said end portions relative to each other. An elongated flexible drive shaft may be operatively connected to the drive wheel, for transferring manual or motor generated power from a location remote from the restriction member. In its simplest embodiment, the drive wheel may comprise a pulley in frictional engagement with the restriction member. As an alternative, a gear rack may be formed on at least one of the end portions of the restriction member and the drive wheel may comprise a gear wheel in mesh with the gear rack. Other suitable known or conventional mechanisms may also or alternatively be used as the adjustment device.

The movement transferring member may alternatively comprise at least one cylinder and a piston, which is movable therein and is connected to one of the end portions of the restriction member, the piston being operable to longitudinally displace the one end portion of the restriction member relative to the other end portion of the restriction member. Alternatively, the movement transferring means may comprise two interconnected cylinders and two pistons in the respective cylinders connected to said end portions, respectively, of the restriction member, the pistons being operable to longitudinally displace the end portions of the restriction member relative to each other. Other known or conventional devices also or alternatively can be used as the movement transferring member.

A motor, which is fixed relative to the main portion of the restriction member and has a rotating drive shaft operatively connected to the movement transferring member, may be positioned relative to the elongated restriction member such that the drive shaft extends transverse thereto. Alternatively, the motor may be positioned relative to the elongated restriction member such that the drive shaft extends substantially tangentially to the loop of the restriction member.

In another embodiment utilizing the first adjustment principle, the elongated restriction member is longitudinally resilient and the adjustment device comprises a contraction device for longitudinally contracting the resilient restriction member. Preferably, the elongated restriction member comprises a substantially nonresilient main portion and an end portion forming an elongated helical spring, which is contractible by the contraction device. The contraction device may suitably comprise an elongated flexible pulling member connected to the main portion of the restriction member and extending through the helical spring to contract the helical spring against an arresting member, which is fixed relative to the main portion of the restriction member. The pulling member may extend in an elongated tube joined at one end thereof to the arresting member, so that a motor remote from the restriction member may be attached to the other end of the elongated tube and pulls the pulling member through the tube to contract the helical spring.

In yet another embodiment utilizing the first adjustment principle, the elongated restriction member comprises an elongated helical spring having a free end, and a body to which the spring is nonrotatably secured at its opposite end. The adjustment device rotates the helical spring in one direction to enlarge the coils of the helical spring to longitudinally contract the spring and to rotate the spring in the opposite direction to reduce the size of the coils of the spring to longitudinally extend the spring. As a preferred alternative, the restriction member comprises a further elongated helical spring having a free end and nonrotatably secured to the body at its opposite end, and the adjustment device comprises a drive shaft having two opposite end portions connected to the springs, respectively, at their free ends, the helical coils forming left and right hand helices, respectively. The adjustment device may alternatively comprise a gearing having an input shaft and two opposite aligned output shafts connected to the helical springs, respectively, at their free ends, the input shaft being connected to said output shafts so that the output shafts rotate in the opposite directions upon rotation of the input shaft, the helical coils forming the same helices.

In accordance with a second adjustment principle, the adjustment device mechanically adjusts the restriction member so that at least a portion of a radially innermost circumferential confinement surface formed by the restriction member is substantially radially displaced.

In one embodiment utilizing the second adjustment principle, the restriction member comprises an elongated voltage responsive element forming part of the confinement surface and capable of bending into a bow in response to a voltage applied across the element, the radius of curvature of the bow being adjustable by changing the level of the voltage.

In another embodiment utilizing the second adjustment principle, the adjustment device changes the diameter of an elastic annular element of the restriction member, which forms the confinement surface. Preferably, the forming means comprises a substantially rigid outer annular element coaxially surrounding the elastic annular element, and the adjustment device comprises means for pulling the elastic annular element radially outwardly towards the outer annular element to expand the elastic annular element. For example, the pulling means may comprise a plurality of threads secured to the elastic annular element along the circumference thereof and running from the elastic annular element via guide members attached to the outer annular element.

In yet another embodiment utilizing the second adjustment principle, the forming means comprises a substantially rigid outer annular element, and the restriction member comprises an elongated helical spring extending internally along the outer annular element and contacting the latter. The helical spring forms part of the circumferential confinement surface and has a free end. The restriction member further comprises a body to which the spring is nonrotatably secured at its opposite end. The adjustment device rotates the helical spring in one direction to enlarge the coils of the spring to contract the circumferential confinement surface and rotates the spring in the opposite direction to reduce the size of the coils of the spring to expand the circumferential confinement surface. As an alternative, which is preferred, the restriction member comprises two elongated helical springs forming part of the circumferential confinement surface and connected to the body of the restriction member. The adjustment device rotates each spring in one direction to enlarge the coils of the spring to contract the circumferential confinement surface and rotates the spring in the opposite direction to reduce the size of the coils of the spring to expand the circumferential confinement surface.

In accordance with a third adjustment principle, the restriction member comprises at least two separate elements, at least one of which is pivoted so that it may turn in a plane in which the the restriction member extends, and the adjustment device turns the pivoted element to change the size of the restriction opening. Preferably, the restriction member comprises a plurality of separate pivoted elements disposed in series, each pivoted element being pivoted in the plane, and the adjustment device turns all of the pivoted elements to change the size of the restriction opening. For example, the pivoted elements may comprise lamellae arranged like the conventional adjustable aperture mechanism of a camera.

In accordance with a fourth adjustment principle, the adjustment device folds at least two foldable frame elements of the restriction member towards each other. Preferably, the foldable frame elements comprise two substantially or partly semi-circular frame elements which are hinged together so that the semi-circular elements are swingable relative to each other from a fully open state in which they form part of a circle to a fully folded state in which they form part of a semi-circle. The same principal may be used with the swingable parts mounted together in one end and not in the other end. Alternatively, the restriction device may comprises two preferable rigid articulated clamping elements positioned on opposite or different sides of the colon or rectum, and the adjustment device turns the clamping elements toward each other to clamp the colon or rectum between the clamping elements, thereby restricting the fecal passageway in the colon or rectum.

In accordance with a fifth adjustment principle, the adjustment device turns the restriction member around a longitudinal extension thereof, the elongated restriction member being elastic and varying in thickness as seen in a cross-section therethrough. Suitably, the elongated restriction member comprises an elastic belt.

In accordance with a sixth adjustment principle, the adjustment device changes the size of the restriction opening such that the outer circumferential confinement surface of the restriction member is changed.

In accordance with a seventh adjustment principle, the adjustment device changes the size of the restriction opening such that the outer circumferential confinement surface of the restriction member is unchanged.

In accordance with an eighth adjustment principle, the elongated restriction member may be flexible, and the adjustment device pulls a first portion of the flexible restriction member from a second portion of the flexible restriction member opposite the first portion in the loop to squeeze the colon or rectum between the opposite lengths of the elongated flexible restriction member to restrict the fecal passageway in the colon or rectum.

In accordance with a ninth adjustment principle, the restriction device comprises at least two elements on opposite or different sides of the colon or rectum, and the adjustment device decreases the distance between the elements to squeeze. the colon or rectum between the elements, thereby restricting the fecal passageway in the colon or rectum. It is also possible to use only one element and squeeze the colon or rectum against human bone or tissue. The elements above may as well as all the restriction members mentioned in this application be everything from rigid to soft.

In accordance with a tenth adjustment principle, the restriction device bends or rotates a portion of colon or rectum to restrict the fecal passageway in the same. For example, the restriction device may comprise at least two bending members, such as cylindrical or hour-glass shaped rollers, positioned on opposite or different sides of the colon or rectum and displaced relative to each other along the colon or rectum, and the adjustment device may move the bending members against the colon or rectum to bend it to restrict the fecal passageway in the colon or rectum. Suitably, the displacement members may comprise rollers. The restriction device may also rotate a portion of the colon or rectum. The bending or rotating members may have any shape or form and be either hydraulic or non-inflatable.

Alternatively, the two bending members one placed more distal than the other may be rotated in opposite direction relative to each other. With interconnecting material for example flexible bands between the holding members a restriction will occur between the bending members when they are rotated.

The restriction device may in all applicable embodiments take any shape and be either hydraulic or non-inflatable.

In all of the above-described embodiments the adjustment device is conveniently operated by an operation device, which may comprise a servo means and/or a motor, preferably a reversible electric motor. The motor may be fixed directly to or be placed in association with the restriction device, or alternatively be located remote from the restriction device, advantageously in the abdomen or pelvic region or subcutaneously or in the retroperitoneum of the patient. In the latter alternative the motor is advantageously connected to the adjustment device by a flexible power transmission conduit to permit a suitable positioning of the motor in the abdomen of the patient. The motor may be manually activatable, for example by an implanted switch.

In some of the above described embodiments of the invention, however, the adjustment device may conveniently be operable by a hydraulic operation device, which preferably is manually activatable. The hydraulic operation device may advantageously include a hydraulic servo to facilitate manual activation. As an alternative, the hydraulic operation device may be powered by an electric motor, which may be manually activatable or controlled by remote control means. The components of such a hydraulic operation device may be placed in association with the restriction device and/or be located at a suitable place in the abdomen or be implanted subcutaneously.

More specifically, a reservoir may be provided containing a predetermined amount of fluid for supplying the hydraulic operation device with fluid. The reservoir defines a chamber for the predetermined amount of fluid and the hydraulic operation device changes the volume of the chamber. The hydraulic operation device may comprise first and second wall portions of the reservoir, which are displaceable relative to each other to change the volume of the chamber of the reservoir. The first and second wall portions of the reservoir may be designed to be displaceable relative to each other by manual manipulation thereof, preferably to permit manual pushing, pulling or rotation of any of the wall portions in one direction. Alternatively, the wall portions may be displaceable relative to each other by magnetic means (such as a permanent magnet and magnetic material reed switch, or other known or conventional magnetic devices), hydraulic means or electrical control means such as an electric motor. The magnetic means, hydraulic means, or electrical control means may all be activated by manual manipulation, preferably using a subcutaneously located manually manipulatable means. This control may be indirect, for example via a switch.

The hydraulic operation device may operate the adjustment device with fluid from the reservoir in response to a predetermined first displacement of the first wall portion of the reservoir relative to the second wall portion of the reservoir, to adjust the restriction device to open the fecal passageway, and to operate the adjustment device with fluid from the reservoir in response to a predetermined second displacement of the first wall portion of the reservoir relative to the second wall portion of the reservoir, to adjust the restriction device to close the fecal passageway. In this embodiment, no pump is used, only the volume of the reservoir is varied. This is of great advantage compared to the solution described below when a pump is used to pump fluid between the reservoir and the adjustment device because there is no need for a non-return valve and it is still possible to have fluid going both to and from the reservoir.

As an alternative, the hydraulic operation means may comprise a pump for pumping fluid between the reservoir and the adjustment device. The pump may pump fluid both to and away from the adjustment device. A mechanical manual solution is proposed in which it is possible to pump in both directions just by pushing an activating member in one direction. Another alternative is a pump pumping in only one direction and an adjustable valve to change the direction of fluid to either increase or decrease the amount of fluid in the reservoir.

This valve may be manipulated manually, mechanically, electrically, magnetically, or hydraulically. Any kind of motor could of course be used for all the different operations as well as wireless remote solutions. The pump may comprise a first activation member for activating the pump to pump fluid from the reservoir to the adjustment device and a second activation member for activating the pump to pump fluid from the adjustment device to the reservoir. The activation members may be operable by manual manipulation, preferably to permit manual pushing, pulling or rotating thereof in one direction. Suitably, at least one of the activation members is adapted to operate when subjected to an external pressure exceeding a predetermined magnitude.

Alternatively, at least one of the first and second activating members may be operable by magnetic means, hydraulic means or electrical control means such as an electric motor. The magnetic means, hydraulic means, or electrical control means may all be activated by manual manipulating means preferably located subcutaneously. This activation may be indirect, for example via a switch.

Advantageously, especially when manual manipulation means are used, a servo means could be used. With servo means less force is needed for operating the adjustment device. The term “servo means” encompasses the normal definition of a servo mechanism, i.e. an automatic device that controls large amounts of power by means of very small amounts of power, but may alternatively or additionally encompass the definition of a mechanism that transfers a weak force acting on a moving element having a long stroke into a strong force acting on another moving element having a short stroke. The servo means may comprise a motor, preferably an electric motor, which may be reversible.

Alternatively, a reverse servo may be employed. The term “reverse servo” is to be understood as a mechanism that transfers a strong force acting on a moving element having a short stroke into a weak force acting on another moving element having a long stroke; i.e. the opposite function of the above-defined alternative mechanism of a normal servo mechanism. A first closed hydraulic system that controls another closed hydraulic system in which hydraulic means of the adjustment device is incorporated may be used. Minor changes in the amount of fluid in a smaller reservoir of the first system could then be transferred by the reverse servo into major changes in the amount of fluid in a larger reservoir in the second system. In consequence, the change of volume in the larger reservoir of the second system affects the hydraulic means of the adjustment device. For example, a short stroke that decreases the volume of the smaller reservoir will cause the larger reservoir to supply the adjustment device with a large amount of hydraulic fluid, which in turn results in a long mechanical adjustment stroke on the restriction device. The great advantage of using such a reverse servo is that the larger volume system could be placed inside the abdomen or retroperitoneum where there is more space and still it would be possible to use manual manipulation means of the smaller system subcutaneously. The smaller reservoir could be controlled directly or indirectly by a fluid supply means. The fluid supply means may include another small reservoir, which may be placed subcutaneously and may be activated by manual manipulation means. Both the servo and reverse servo may be used in connection with all of the various components and solutions described in the present specification.

Preferably, the reverse servo comprises hydraulic means and a main fluid supply reservoir and eventually an additional fluid supply reservoir. Both reservoirs define a chamber containing hydraulic fluid, and the hydraulic means comprises first and second wall portions of the main fluid supply reservoir, which are displaceable relative to each other to change the volume of the chamber of the main fluid supply reservoir. The hydraulic means may control the adjustment device indirectly, e.g. via an increased amount of fluid in the main fluid supply reservoir, in response to a predetermined first displacement of the first wall portion of any of the reservoirs relative to the second wall portion of the reservoir to close the fecal passageway, and to control the adjustment device in response to a second displacement of the first wall portion of any reservoir relative to the second wall portion, to indirectly adjust the restriction device to open the fecal passageway. The wall portions of the reservoirs may be designed to be displaceable relative to each other by manual manipulation thereof or be displaceable relative to each other by manually pushing, pulling, or rotating any of the wall portions of the reservoir in one direction. Alternatively, the wall portions of the main fluid supply reservoir may be displaceable relative to each other by magnetic means, hydraulic means or electric control means including an electric motor.

The magnetic means, hydraulic means, or electrical control means may all be activated by manually manipulated means preferably located subcutaneously. This control may be indirect for example via a switch.

Even in the broadest embodiment the adjustment device may comprise a servo means. The servo means may comprise a hydraulic operation means, an electrical control means, a magnetic means, mechanical means or a manual manipulation means. The hydraulic operation means, electrical control means, mechanical means or magnetic means may be activated by manual manipulating means. Using a servo system will save the use of force when adjusting the adjustment device, which may be of importance in many applications, for example when a battery cannot put out enough current although the total energy in the battery is more than enough to power the system.

In accordance with a preferred embodiment of the invention, the apparatus comprises implantable electrical components including at least one, or only one single voltage level guard and a capacitor or accumulator, wherein the charge and discharge of the capacitor or accumulator is controlled by use of the voltage level guard. As a result, there is no need for any implanted current detector and/or charge level detector for the control of the capacitor, which makes the apparatus simple and reliable.

All solutions may be controlled by a wireless remote control for controlling the adjustment device. The remote control may advantageously be capable of obtaining information related to the fecal passageway or the pressure against the restriction device or colon or rectum or other important physical parameters and of commanding the adjustment device to adjust the restriction device in response to obtained information. With the wireless remote control the apparatus is conveniently controlled by the patient when he so desires, which is of great advantage compared to the prior art procedures. With the remote control the apparatus is conveniently controlled to adjust the implanted restriction device to release the fecal passageway when the patient wants to relieve himself or herself.

The apparatus may further comprise a pressure sensor for directly or indirectly sensing the pressure against the restriction device and the restriction device may control the blood flow in response to signals from the pressure sensor. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor. The adjustment device preferably non-invasively adjusts the restriction device to change the size of the cross-sectional area.

The adjustment device and/or other energy consuming components of the apparatus may be energized with wirelessly transmitted energy from outside the patient's body or be powered by an implanted battery or accumulator.

The apparatus may further comprise an implanted energy transforming device for transferring wireless energy directly or indirectly into kinetic energy for operation of the restriction device. The energy transforming device may, preferably directly, transform the wireless energy in the form of sound waves into electric energy for operation of the restriction device. Suitably the energy transforming device comprises a capacitor adapted to produce electric pulses from the transformed electric energy.

The apparatus may further comprise an energy transfer means for wireless transfer of energy from outside the patient's body to the adjustment device and/or other energy consuming implantable components of the apparatus. The energy transfer means may be adapted to intermittently transfer the energy, preferably electric energy, in the form of a train of energy pulses for direct use in connection with the energizing of the energy consuming components of the apparatus. An implanted capacitor having a capacity less than 0.1 μF may be used for producing the train of energy pulses.

A motor may be implanted for operating the adjustment device, wherein the energy transfer means is adapted to directly power the motor with transferred energy. Alternatively, or in combination with the motor, a pump may be implanted for operating the adjustment device, wherein the energy transfer means is adapted to transfer wireless energy in the form of electromagnetic waves for direct power of the pump. Preferably, the pump is not a plunger type of pump, but may comprise a peristaltic or membrane pump.

The energy transfer means preferably transfers wireless energy in the form of electromagnetic waves. However, for safety radio waves may be excluded.

Alternatively, the energy transferred by the energy transfer means may comprise an electric field or a magnetic field.

Most preferred, the energy transferred by the energy transfer means comprises a signal.

Preferably, the wireless remote control comprises a separate signal transmitter or receiver and a signal receiver or transmitter implanted in the patient. For example, the signal transmitter and signal receiver may transmit and receive a signal in the form of digital pulses, which may comprise a magnetic or electric field. Alternatively, which is preferred, the signal transmitter and signal receiver may transmit and receive an electromagnetic wave signal, a sound wave signal or a carrier wave signal for a remote control signal. The receiver may comprise an implanted control unit for controlling the adjustment device in response to a control signal from the signal transmitter.

The apparatus may further comprise an implanted energizer unit for providing energy to energy consuming implanted components of the apparatus, such as electronic circuits and/or a motor for operating the adjustment device. The apparatus may comprise an external energy transmitter for transmitting wireless energy, wherein the energizer unit is adapted to transform the wireless energy into electric energy. An implanted electric motor may operate the adjustment device and the energizer unit may be adapted to power the electric motor with the electric energy transformed from the wireless energy.

The energizer unit may comprise a battery and a switch operable by the wireless energy transmitted by the external transmitter, for connecting the battery to the implanted energy consuming components of the apparatus in an “on” mode when the switch is powered by the wireless energy and to keep the battery disconnected from the energy consuming components in a “standby” mode when the switch is not powered.

The control unit may power such an implanted motor with energy provided by the energizer unit in response to a control signal received from the signal transmitter. Any known or conventional signal transmitter or signal receiver that is suitable for use with a human or mammal patient may be provided as the signal transmitter or signal receiver of the invention.

Generally, all of the above signals may comprise electromagnetic waves, such as infrared light, visible light, laser light, micro waves, or sound waves, such as ultrasonic waves or infrasonic waves, or any other type of wave signals. The signals may. also comprise electric or magnetic fields, or pulses. All of the above-mentioned signals may comprise digital signals. The signals may be carried by a carrier wave signal, which in an alternative embodiment may be the same signal as the wireless energy signal. Preferably a digital control signal may be carried by an electromagnetic wave signal. The carrier wave or control signal may be amplitude or frequency modulated.

The motor may be any type of motor, such as a pneumatic, hydraulic or electric motor and the energizer unit may power the motor with pressurized gas or liquid, or electric energy, depending on the type of motor. Where the motor is an electric motor, it may power pneumatic or hydraulic equipment.

The energizer unit may comprise a power supply and the control unit may power the motor with energy from the power supply. Preferably, the power supply is an electric power supply, such as a battery, and the motor is an electric motor. In this case, the battery also continuously powers at least part of the circuitry of the signal receiver in a standby mode between the adjustments, in order to keep the signal receiver prepared for receiving signals transmitted from the signal transmitter.

The energizer unit may transform energy from the control signal, as the control signal is transmitted to the signal receiver, into electric energy for powering the implanted electronic components. For example, the energizer unit may transform the energy from the control signal into a direct or alternating current.

In case there is an implanted electric motor for operating the adjustment device the energizer unit may also power the motor with the transformed energy. Advantageously, the control unit directly powers the electric motor with electric energy, as the energizer unit transforms the signal energy into the electric energy. This embodiment is particularly simple and does not require any recurrent invasive measures for exchanging empty power supplies, such as batteries, that is required in the first embodiment described above. The motor may also be directly powered with wirelessly transmitted electromagnetic or magnetic energy in the form of signals, as the energy is transmitted. All the various functions of the motor and associated components described in the present specification may be used where applicable.

For adjustment devices of the type that requires more, but still relatively low, power for its operation, the energizer unit may comprise a rechargeable electric power supply for storing the electric energy obtained and the control unit may power the electric motor with energy from the rechargeable electric power supply in response to a control signal received from the signal transmitter. In this case, the rechargeable power supply can be charged over a relatively long time (e.g. a few seconds up to a half hour) without powering the electric motor.

The electric power supply suitably comprises an inexpensive simple capacitor. In this case, the electric motor may be a stepping motor. In all embodiments the motor may preferably be able to perform a reversing function.

The signal transmitter may transmit an electromagnetic signal and the energizer unit may draw radiant energy from the electromagnetic wave signal, as the latter is transmitted to the signal receiver, and transform the radiant energy into electric energy.

Alternatively, the energizer unit may comprise a battery or an accumulator, an electrically operable switch adapted to connect the battery to the signal receiver in an on mode when the switch is powered and to keep the battery disconnected from the signal receiver in a standby mode when the switch is unpowered, and a rechargeable electric power supply for powering the switch. The control unit may power the electric motor with energy from the battery in response to a control signal received from the signal transmitter, when the switch is in its on mode. Advantageously, the energizer unit may transform wave energy from the control signal, as the latter is transmitted to the signal receiver, into a current for charging the rechargeable electric power supply, which suitably is a capacitor. Energy from the power supply is then used to change the switch from off (standby mode) to on. This embodiment is suited for adjustment devices of the type that require relatively high power for their operation and has the advantage that the electronic circuitry of the signal receiver does not have to be powered by the battery between adjustments. As a result, the life-time of the battery can be significantly prolonged. The switch may be switched with magnetic, manual or electric energy.

As an example, the signal transmitter may transmit an electromagnetic wave signal and the energizer unit may draw radiant energy from the electromagnetic wave signal, as this is transmitted to the signal receiver, and may transform the radiant energy into said current. The energizer unit suitably comprises a coil of the signal receiver for inducing an alternating current as the electromagnetic wave signal is transmitted through the coil and a rectifier for rectifying the alternating current. The rectified current is used for charging the rechargeable power source.

Alternatively, the signal transmitter and receiver may solely be used for a control signal and a further pair of signal transmitter and receiver may be provided for transferring signal energy to. implanted components. By such a double system of signal transmitters and receivers the advantage is obtained that the two systems can be designed optimally for their respective purposes, namely to transmit a control signal and to transfer energy from an energy signal.

Accordingly, the apparatus may further comprise an external energy transmitter for transmitting wireless energy, wherein the energizer unit comprises a battery and an operable switch for connecting the battery to the signal receiver in an on mode when the switch is powered and for keeping the battery disconnected from the signal receiver in a standby mode when the switch is unpowered, and the external energy transmitter powers the switch. Suitably, the energy transmitter may directly power the switch with the wireless energy to switch into the on mode. As should be realized by a skilled person, in many of the above-described embodiments the adjustment device may be operated by control means or manual manipulation means implanted under the skin of the patient, such as a pump, an electrical switch or a mechanical movement transferring means. In the manual embodiment it is not necessary to use a motor for operating the adjustment device.

In embodiments including hydraulic transmission means, an injection port connected to the hydraulic means may be provided for enabling, normally single, once-and-for-all, calibration of the amount of fluid in the hydraulic system.

In all embodiments a motor may be operatively connected to the adjustment device. A reversing device may be implanted in the patient for reversing the motor.

The adjustment device may be adapted to hydraulically adjust the restriction device by using hydraulic means which is devoid of hydraulic fluid of the kind having a viscosity that substantially increases when exposed to heat or a magnetic field, i.e. the hydraulic fluid would not become more viscous when exposed to heat or influenced by magnetic forces. A pressure sensor may be used to directly or indirectly sense the pressure against the restriction device or the colon or rectum to avoid occurrence of necrosis of the human tissue. The restriction device may be controlled in response to signals from the pressure sensor. The motor which preferably is used to adjust the restriction device must then be capable of performing a reversible function, that is to say, be capable of reversing the driving direction of the motor.

Preferably the adjustment device is directly energized with wirelessly transmitted energy from outside the patient's body. The implanted energy transforming device transforms wireless energy directly or indirectly into kinetic energy for operation of the restriction device. In accordance with another embodiment it would also be possible to use an implanted accumulator or battery and control this implanted energy source from outside the patient's body to supply energy to the adjustment device or other energy consuming parts of the implanted apparatus.

All the above-described various components, such as the motor, pump and capacitor, may be combined in the different embodiments where applicable. Also the various functions described in connection with the above embodiments may be used in different applications, where applicable.

The invention also provides a method for treating a patient suffering from anal incontinence comprising surgically implanting in the body of the patient an adjustable restriction device which directly engages the colon or rectum of the patient like an artificial sphincter around the fecal passageway therein, normally closed, and when desired, mechanically adjusting the restriction device to temporarily open the fecal passageway.

The adjustable restriction device preferably is implanted in the base or prolongation of the patient's rectum. It would also be possible to use two or more restricting devices engaging the colon or rectum.

The invention further provides a method for treating anal incontinence, comprising the steps of placing at least two laparascopical trocars in the body of a patient suffering from anal incontinence, inserting a dissecting tool through the trocars and dissecting an area of the colon or rectum in the abdominal or pelvic or retroperitoneal surroundings, placing at least one adjustable restriction device in the dissected area engaging the rectum or colon, adjusting the restriction device to normally restrict the fecal passageway in the rectum or colon, and adjusting the restriction device to open the fecal passageway when the patient wants to relieve himself or herself. A mechanically adjustable restriction device may be used when practicing this method, preferably in a non-manual manner, i.e. without touching subcutaneously implanted components of the apparatus.

The method may further comprise implanting a source of energy in the patient and providing a control device for controlling the source of energy from outside the patient's body to supply energy to the restriction device.

### Aspect 447A, B, C Data_Packet_Encryption—Implant, External, Method

According to an aspect, an implantable medical device configured to receive remote instructions from an external system is provided. The implantable medical device comprises a wireless receiver configured to receive wirelessly transmitted data packets from the external system, and a computing unit configured to verify the electronic signature, and use a checksum provided in the data packet to verify the integrity of the instructions.

According to an embodiment, the computing unit is configured to decrypt the data packet.

According to an embodiment, the computing unit is configured to use the checksum to verify that the bit stream making up the instructions is unchanged.

According to an embodiment, the wireless receiver is part of a wireless transceiver.

According to an embodiment, the computing unit comprises a memory unit configured to store electronic signatures, and wherein the computing unit is configured to verify the electronic signature my comparing the electronic signature with the electronic signatures stored in the memory unit.

According to an embodiment, the implantable medical device comprises a control program configured to control at least one function of the implantable medical device, and wherein computing unit is configured to alter the control program on the basis of the received instructions.

According to an embodiment, the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.

According to an embodiment, the computing unit comprises a memory unit configured to store parameter values, and wherein the method further comprises the step of verifying that the instructions for altering the at least one parameter will result in the at least one parameter being updated to a parameter value comprised in the set of stored parameter values.

According to an embodiment, the implantable medical device comprises a central unit, comprising at least one of a wireless receiver and a wireless transceiver, and a security module connected to the central unit, wherein the implantable medical device is configured to transfer the data packet from the central unit to the security module and wherein the security module is configured to performing at least a portion of at least one of the decryption and the signature verification.

According to an embodiment, the security module comprises a set of rules for accepting communication from the central unit, and wherein the security module is configured to verify compliance with the set of rules.

According to an embodiment, wireless receiver or wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.

According to an embodiment, wherein the implantable medical device is configured to at least one of decrypting the data packet and verifying the electronic signature using a private key of the implantable medical device.

According to an embodiment, the private key is a non-extractable key.

According to an embodiment, the implantable medical device is configured to perform a proof of possession operation comprising transmitting, from the implantable medical device to the external system, a query based on a public key associated with the private key of the external system, receiving, at the implantable medical device, a response based on the possession of the private key in the external system, and verifying that the response based on the possession of the private key matches the query based on a public key.

According to an embodiment, the implantable medical device is configured to communicate with the external system independently of time.

According to an embodiment, the private key is provided in the implantable medical device by the manufacturer of the implantable medical device.

According to an embodiment, the private key is stored as hardware or software in the implantable medical device.

According to an embodiment, the implantable medical device is configured to verify a first electronic signature made using at least one of a first key and a second key, and verifying a second electronic signature made using at least one of a first key and a second key.

According to an embodiment, at least one of the first and second keys is a private key.

According to an embodiment, the first and second keys are different.

According to an embodiment, the first and second keys comprises at least one common element.

According to an embodiment, the implantable medical device is configured to verify a first electronic signature to allow communication from the external system to the implantable medical device, and verify a second electronic signature to allow an instruction received in the communication to alter the control program running on the implantable medical device.

According to an embodiment, the first electronic signature is an electronic signature linked to the user of the implantable medical device and the second electronic signature is an electronic signature linked to a healthcare provider.

According to an embodiment, only a portion of the private key is needed to at least one of. decrypt the data packet and verify the electronic signature.

According to an embodiment, the implantable medical device trusts any external device holding the private key.

According to an embodiment, the implantable medical device is configured to receive the data packet comprising at least one instruction signed by a private key of the external system, and a public key including information about which root have created the public key.

According to an embodiment, the implantable medical device is configured to accept communication from an external system based on at least one password being provided to the implantable medical device.

According to an embodiment, the implantable medical device is configured to accept communication from an external system based on two passwords being provided to the implantable medical device.

According to an embodiment, the implantable medical device is configured to accept communication from an external system based on one patient password and one healthcare provider passwords being provided to the implantable medical device.

According to an aspect, an external system for providing remote instructions to an implantable medical device is provided. The external system is configured to provide instructions to be transmitted to the implantable medical device, derive a checksum from the instructions, electronically sign the instructions and the checksum, form a data packet from the instructions, the electronic signature and the checksum, wherein the external system comprises a wireless transmitter configured to wirelessly send the data packet to the implantable medical device.

According to an embodiment, the external system is further configured to encrypt the data packet at the external system.

According to an embodiment, the wireless transmitter is part of a wireless transceiver comprised in the external system.

According to an embodiment, the external system comprises a first external device and a second external device, and wherein the first external device is configured to transmit the data packet to the second external device, and wherein the second external device is configured to transmit the data packet wirelessly to the implantable medical device without changing the data packet.

According to an embodiment, the external system comprises a first external device and a second external device, and wherein the first external device is configured to transmit the data packet to the second external device, and wherein the second external device is configured to transmit the data packet wirelessly to the implantable medical device without full decryption of the data packet.

According to an embodiment, the external system is configured to transmit at least one instruction for altering the control program of the implantable medical device, to the implantable medical device.

According to an embodiment, the external system is configured to provide at least one instruction to the implantable medical device for altering at least one parameter for affecting the control of the implantable medical device.

According to an embodiment, the external system is configured to provide at least one instruction for updating at least one parameter of the control program to a parameter value comprised in a set of parameter values stored in the implantable medical device.

According to an embodiment, the first external device is configured to send the data packet from the first external device to the second external device using a first network protocol and send the data packet from the second external device to the implantable medical device using a second network protocol.

According to an embodiment, the first external device is configured to send the data packet from the first external device to the second external device using wired communication and send the data packet from the second external device to the implantable medical device using wireless communication.

According to an embodiment, the first external device is configured to wirelessly send the data packet from the first external device to the second external device using a first network protocol, and wirelessly send the data packet from the second external device to the implantable medical device using a second network protocol.

According to an embodiment, the first external device is configured to wirelessly send the data packet from the first external device to the second external device using a first frequency band, and wirelessly send the data packet from the second external device to the implantable medical device using a second frequency band.

According to an embodiment, the first external device is configured to wirelessly send the data packet from the first external device to the second external device using a first wireless technology, and wirelessly send the data packet from the second external device to the implantable medical device using a second wireless technology.

According to an embodiment, the external system is configured to electronically sign the instructions at the external system using a key of the external system.

According to an embodiment, the key is a non-extractable key.

According to an embodiment, the second external device is configured to perform a proof of possession operation comprising the steps of transmitting, form the first external device to the second external device, a query based on a public key associated with the private of the external system, receiving, at the second external device, a response based on the possession of the private key in the first external device, and verifying that the response based on the possession of the private key matches the query based on a public key.

According to an embodiment, the first external device is configured to form the data packet and electronically sign the instruction using a first private key, and the second external device is configured to: receive the data packet from the first external device, verify that the first external device is a trusted transmitter, in response to the verification, electronically sign the data packet using a second private key, and transmit the data packet from the second external device to the medical implant.

According to an embodiment, the checksum is configured to verify that no changes have been made to the bit stream forming the instructions.

According to an embodiment, the first external device is configured to at least one of: electronically sign the instructions and encrypt the data packet using a key placed on a key device external to the first external device.

According to an embodiment, the external system further comprises a key device configured to hold at least one private key.

According to an embodiment, the key device comprises a wireless transmitter for wirelessly transmitting the at least one private key or a signal based on the private key, to the first external device.

According to an embodiment, the second external device is configured to at least one of: electronically sign the instructions and encrypt the data packet using a key placed on a key device external to the second external device.

According to an embodiment, the external system further comprises a second key device configured to hold at least one second private key.

According to an embodiment, the second key device comprises a wireless transmitter for wirelessly transmitting the at least one private key or a signal based on the private key to the second external device.

According to an embodiment, the external system further comprises a second key device comprising a wireless transmitter for wirelessly transmitting at least one second private key or a signal based on the second private key to the first external device.

According to an embodiment, at least one of the key device and the second key device comprises at least one of: a key card, a wearable device and a handset.

According to an embodiment, the first external device is configured to be unlocked by user credentials provided to the first external device.

According to an embodiment, the first external device is configured to be unlocked by user credentials comprising a username and a password.

According to an embodiment, the first external device is configured to be unlocked by user credentials comprising a PIN-code.

According to an embodiment, the first external device is configured to verify the user credentials by comparing the user credentials with user credentials stored in the first external device.

According to an embodiment, the first external device is configured to verify the user credentials by comparing the user credentials with user credentials stored in the first external device by the manufacturer of the first external device.

According to an embodiment, the first external device is configured verify the user credentials by comparing the user credentials with user credentials stored as hardware or software in the first external device.

According to an embodiment, the first external device is configured verify the user credentials by communicating with a remote server.

According to an embodiment, the second external device is configured to be unlocked by user credentials provided to the second external device.

According to an embodiment, the first external device is configured to be unlocked by user credentials comprising a username and a password.

According to an embodiment, the first external device is configured to be unlocked by user credentials comprising a PIN-code.

According to an embodiment, the second external device is configured to verify the user credentials by comparing the user credentials with user credentials stored in the second external device.

According to an embodiment, the second external device is configured to verify the user credentials by comparing the user credentials with user credentials stored in the second external device by the manufacturer of the second external device.

According to an embodiment, the second external device is configured verify the user credentials by comparing the user credentials with user credentials stored as hardware or software in the second external device.

According to an embodiment, the second external device is configured verify the user credentials by communicating with a remote server.

According to an embodiment, the external system is configured to function without connection to the Internet.

According to an embodiment, the external system is configured to communicate with the implantable medical device independently of time.

According to an embodiment, the first and second private keys are different.

According to an embodiment, the first and second private keys comprises at least one common element.

According to an embodiment, at least one first and second external device are configured to be unlocked by at least one of the first and second private key.

According to an embodiment, the external system comprises a central server, and wherein the central server is configured to form a data packet from the instructions, the electronic signature and the checksum and further configured to provide the formed data packet to the first external device.

According to an embodiment, the central server can be accessed by at least one healthcare professional, such that the healthcare professional can provide input to the central server for forming the instructions to be sent to the implantable medical device.

According to an embodiment, the central server can be accessed by at least one patient, such that the patient can provide input to the central server for verifying at least one of: the authenticity of the healthcare professional and the correctness of the instructions.

According to an embodiment, the healthcare provider can electronically sign the instructions at the central server.

According to an embodiment, the patient can electronically sign the instructions at the central server.

According to an embodiment, the central server is configured to verify the authenticity of the first and second key and electronically sign the instructions using the first and second key.

According to an embodiment, the second key is a user key, and wherein the external system is configured to use the second key for at least one of approving that communication is transmitted to the implantable medical device, and approving that a healthcare provider prepares an instruction to the implantable medical device.

According to an embodiment, the approval step can be performed by first or second external device.

According to an embodiment, the first key is required to create an instruction to the implantable medical device and the second key is required to transmit the created instruction to the implantable medical device.

According to an embodiment, at least one of the first and second external device comprises an input button configured to be used for verifying user presence.

According to an embodiment, the input button con be configured to replace at least one of input of at least one key to at least one of the first and second external device, and input of credentials into at least one of the first and second external device.

According to an embodiment, the input button is configured to replace the second key.

According to an embodiment, the external system is configured to transmit the data packet to the implantable medical device, and wherein the data packet comprises at least one instruction signed by a first key and a public key including information about which root have created the public key.

According to an embodiment, at least one of the first and second external device is configured to enable communication with the implantable medical device based on at least one password being provided to at least one of the first and second external device.

According to an embodiment, at least one of the first and second external device is configured to enable communication with the implantable medical device based on two passwords being provided to at least one of the first and second external device.

According to an embodiment, at least one of the first and second external device is configured to enable communication with the implantable medical device based on one patient password and one healthcare provider passwords being provided to at least one of the first and second external device.

According to an embodiment, at least one of the first and second external devices are configured to perform a verification query operation with at least one of the first and second key device, the verification query operation comprising transmitting, from the first or second external devices, a query comprising a computational challenge to at least one of the first and second key device, receiving, at the first or second external devices, a response based on the transmitted computational challenge, and verifying, at the first or second external devices, the received response.

According to an embodiment, at least one of the first and second external devices are configured to perform a verification query operation in the form of a proof of possession operation comprising receiving a public key of at least one of the first and second key devices, the public key being associated with a private key of the first or second key device, transmitting, from at least one of the first and second external devices, a computational challenge to the first or second key device, based on the public key received from the first or second key device, receiving a response from the first or second key device based on the possession of the private key in the first or second key device, and verifying that the response based on the possession of the private key matches the query based on a public key.

A medical system comprising the external system according to any one of the preceding embodiments and an implantable medical device is also provided.

A method corresponding to any one of the embodiments of aspects 447A and/or 447B is provided.

### Aspect 447D Single-Use_Codes_Encryption

A method of providing remote instructions from an external system to an implantable medical device, wherein the implantable medical device comprises a list of codes and the external system comprises a list of codes, is provided. The method comprises encrypting the instructions at the external system using a code from a position on the list of codes, wirelessly sending the encrypted instructions to the implantable medical device, and decrypting, at the implantable medical device, the instructions using a code from a position on the list of codes.

According to an embodiment, the method further comprises the steps of wirelessly sending position information from the external device to the implantable medical device, and using the information at the implantable medical device for selecting the code from the list of codes.

According to an embodiment, the step of encrypting, at the external system, the instructions using a code from a position on the list of codes comprises selecting the code on a current position on the list of codes, wherein the method further comprises the step of updating the current position to a new current position after using the code.

According to an embodiment, the step of decrypting, at the implantable medical device, the instructions using a code from a position on the list of codes comprises selecting the code on a current position on the list of codes, wherein the method further comprises the step of updating the current position to a new current position after using the code.

According to an embodiment, the current position comprises a number and wherein the step of updating the current position comprises updating the number to a sequential number.

According to an embodiment, the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without changing the encrypted instructions.

According to an embodiment, the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without full decryption.

According to an embodiment, the implantable medical device comprises a control program configured to control at least one function of the implantable medical device, and wherein the method further comprises altering the control program on the basis of the received instructions.

According to an embodiment, the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.

According to an embodiment, the implantable medical device comprises a set of stored parameter values, and wherein the method further comprises the step of verifying that the instructions for altering the at least one parameter will result in the at least one parameter being updated to a parameter value comprised in the set of stored parameter values.

According to an embodiment, the step of wirelessly sending the encrypted instructions to the implantable medical device comprises wirelessly sending the encrypted instructions from a first external device to a second external device using a first network protocol, and wirelessly sending the encrypted instructions from the second external device to the implantable medical device using a second network protocol.

According to an embodiment, the step of wirelessly sending the encrypted instructions to the implantable medical device comprises wirelessly sending the encrypted instructions from a first external device to a second external device using a first frequency band, and wirelessly sending the encrypted instructions from the second external device to the implantable medical device using a second frequency band.

According to an embodiment, the step of wirelessly sending the encrypted instructions to the implantable medical device comprises wirelessly sending the encrypted instructions from a first external device to a second external device using a first wireless technology, and wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.

According to an embodiment, the implantable medical device comprises a central unit, comprising a wireless transceiver, and a security module connected to the central unit, wherein the step of decrypting, at the implantable medical device, the encrypted instructions, comprises transferring the encrypted instructions from the central unit to the security module, and performing at least a portion of the decryption in the security module.

According to an embodiment, the security module comprises a set of rules for accepting communication from the central unit, and wherein the step of transferring the encrypted instructions from the receiving unit of the implant to the security module comprises verifying compliance with the set of rules.

According to an embodiment, the wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.

According to an embodiment, the step of electronically signing the instructions at the external system comprises electronically signing the instructions at the external system using a private key of the external system.

According to an embodiment, the private key is a non-extractable key.

According to an embodiment, the step of wirelessly sending the encrypted instructions to the implantable medical device comprises wirelessly sending the encrypted instructions from a first external device to a second external device using a first wireless technology, and wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.

### Aspect 454: Dual Remote Controls

A communication system for transmission of data to or from an implantable medical device is provided. The communication system comprises an implantable medical implant, a first remote control comprising a first wireless communication unit configured for wireless transmission of data to or from the implantable medical device, the first remote control being operable by a user, and a second remote control comprising a second wireless communication unit configured for wireless transmission of control commands or data to or from the implantable medical device, and a third communication unit for communicating with a patient display device, the second remote control being inoperable by a user.

According to an embodiment, the first remote control comprises an input device for receiving a first user input, and wherein the first remote control is configured to transmit the first user input to the implantable medical device.

According to an embodiment, the second remote control is configured to receive second user input from the patient display device and to transmit the second user input to the implantable medical implant.

According to an embodiment, the data comprises a control command for the medical implant.

According to an embodiment, at least one of the first wireless communication unit and the second wireless communication unit is configured to send or receive data using near-field magnetic induction.

According to an embodiment, at least one of the first wireless communication unit and the second wireless communication unit comprises a transmitter coil for modulating a magnetic field for transmitting the data, and wherein the implantable medical implant comprises a receiving coil and an NFMI receiver connected to the receiving coil to receive the data.

According to an embodiment, the transmitter coil is configured to modulate a magnetic field, and the NFMI receiver is adapted to measure the magnetic field in the receiving coil.

According to an embodiment, at least one of the first wireless communication unit and the second wireless communication unit is configured to wirelessly charge the medical implant using near-field magnetic induction.

According to an embodiment, the medical implant comprises a coil for receiving wireless energy for charging the implant via near-field magnetic induction.

According to an embodiment, the second and third communication units are configured to transmit and/or receive data using different network protocols.

According to an embodiment, wherein the second and third communication units are configured to transmit and/or receive data using different frequency bands.

According to an embodiment, at least one of the first remote control, the second remote control and the implantable medical device comprises a Bluetooth transceiver.

According to an embodiment, at least one of first remote control, the second remote control and the implantable medical device comprises a UWB transceiver.

According to an embodiment, the network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.

According to an embodiment, the second communication unit has a longer effective range than the third communication unit.

According to an embodiment, the second remote control is configured to communicate with a consumer electronics device.

According to an embodiment, the patient display device comprises the consumer electronics device.

According to an embodiment, the first remote control is configured to control functions of the implantable medical device based on user input to the first remote control.

According to an embodiment, a method corresponding to the communication system according to the previous aspect is provided.

### Aspect 457: Controlling Energy Transfer

According to an aspect, a method for wireless energy transfer from an external energy source located outside the patient to an internal energy receiver located inside the patient, the internal energy receiver being connected to an implantable medical device for supplying received energy thereto, is provided. The method comprises determining an accumulated amount of received energy over a time period, determining a current change in the received energy, determining a control signal reflecting the accumulated received energy and the change in the received energy, and controlling the energy transfer based on the control signal.

According to an embodiment, determining an accumulated amount of received energy is determined by the internal energy receiver.

According to an embodiment, determining a current change is performed by the internal energy receiver.

According to an embodiment, the internal energy receiver comprises a PID regulator for controlling the energy transfer.

According to an embodiment, the PID regulator is implemented in a microcontroller.

According to an embodiment, determining a control signal is performed by the internal energy receiver.

According to an embodiment, the control signal is transmitted to the external energy source, and wherein the external energy source is configured to adjust the transmitted energy base on the control signal.

According to an embodiment, controlling the energy transfer is controlled by the internal energy receiver.

According to an embodiment, controlling the energy transfer is performed by the external energy source.

According to an embodiment, controlling the energy transfer comprises adjusting the energy transfer efficiency.

According to an embodiment, the external device comprises a transmitter coil for modulating a magnetic field for transmitting data or transmitting energy, and wherein the implantable medical implant comprises a receiving coil and an NFMI receiver connected to the receiving coil to receive the data or the energy.

According to an embodiment, at least one of the first wireless communication unit and the second wireless communication unit is configured to wirelessly charge the medical implant using near-field magnetic induction.

According to an embodiment, the medical implant comprises a coil for receiving wireless energy for charging the implant via near-field magnetic induction.

According to an embodiment, the method further comprises receiving energy in pulses according to a pulse pattern, and measuring the received pulse pattern.

According to an embodiment, the method further comprises determining that the pulse pattern deviates from a predefined pulse pattern, and controlling the energy transfer based on the determination.

According to an embodiment, the method further comprises measuring a temperature in the implantable medical device or in the body of the patient, and controlling the energy transfer in response to the measured temperature.

According to an embodiment, the implantable medical device comprises at least one coil connected to a variable impedance, the method further comprising controlling the energy transfer by controlling the variable impedance.

According to an embodiment, the implantable medical device comprises at least one coil having a plurality of windings, wherein the plurality of windings each are connected to a respective variable impedance, the method further comprising controlling the energy transfer by controlling the respective variable impedance individually.

According to an aspect, an implantable medical device, a first remote control and/or a second remote control configured to perform the method according to the previous aspect are provided. A corresponding method is also provided.

### Aspect 453: Voice Control

A method of teaching a voice-controlled medical implant to recognize a voice command is provided. The method comprises inputting a first audio training phrase to the medical implant, when the medical implant is implanted in the body of the patient and creating a transfer function, the transfer function being based on the first audio training phrase, wherein the transfer function is configured to adjust the amplitude of at least one frequency of audio received at the medical device for enhancing audio received at the medical implant to facilitate detection of voice commands. The method further comprises inputting a second audio training phrase to the medical implant, the second audio training phrase comprising the voice command. The voice command comprises an instruction for the control of the medical implant. The method further comprises using the transfer function for generating an enhanced second audio training phrase in the medical implant, and associating the enhanced second audio training phrase with the instruction for the control of the medical implant.

In an embodiment, adjusting the amplitude comprises at least one of: filtering, cancelling and amplifying the at least one frequency.

In an embodiment, at least one of the first and second audio training phrase is a spoken audio training phrase.

In an embodiment, the spoken audio training phrase is spoken by the patient the implant is implanted in.

In an embodiment, the first audio training phrase comprises the voice command.

In an embodiment, the first and second audio training phrases is the same voice command.

In an embodiment, the first and second audio training phrases are different.

In an embodiment, creating the transfer function comprises amplifying frequencies muffled by the location of the medical implant in the body of the patient.

In an embodiment, creating the transfer function comprises filtering or cancelling noise generated by the body.

In an embodiment, the medical implant is configured to receive voice commands related to an instruction for control of the medical implant.

In an embodiment, the voice command relates to at least one of: performing a function of the medical device; using a sensor to measure a parameter relating to a condition of the patient or a condition of the medial implant; and sending or receiving data from the medical implant.

A system corresponding to the preceding aspect is also provided.

### Aspect Large Coil

According to an aspect, a system for wirelessly charging an implantable medical implant, when implanted in a body of a patient, is provided. The system comprises an internal energy receiver comprising a secondary coil, the internal energy receiver being connected to the implantable medical implant and an external energy transmitter comprising a primary coil for wirelessly transmitting energy to the internal energy receiver via the secondary coil, wherein a diameter of the primary coil is larger than a diameter of the secondary coil.

According to an embodiment, the system further comprises an internal controller connected to the internal energy receiver, for controlling the amount of energy received by the internal energy receiver.

According to an embodiment, the internal energy receiver further comprises a measurement unit for measuring a parameter related to the implantable medical implant or the body of the patient.

According to an embodiment, the controller is configured to measure the accumulated energy received by the internal energy receiver over a period of time and to measure a current change in energy received, and to control the energy received based on the accumulated energy and the current change.

According to an embodiment, the controlled comprises a Proportional-Integral-Derivative, PID, regulator for controlling the received energy.

According to an embodiment, the internal energy received comprises a variable impedance.

According to an embodiment, the internal energy receiver is configured to control the resonant frequency by controlling the variable impedance.

According to an embodiment, the controller is configured to vary the variable impedance in response to a measured parameter deviating from a predetermined interval or exceeding a threshold value.

According to an embodiment, the parameter relates to the energy received by the coil over a time period.

According to an embodiment, the measurement unit is configured to measure a parameter related to a change in energy received by the coil.

According to an embodiment, the receiving unit is configured to receive transcutaneously transferred energy in pulses according to a pulse pattern, and wherein the measurement unit is configured to measure a parameter related to the pulse pattern.

According to an embodiment, the receiving unit is configured to receive transcutaneously transferred energy in pulses according to a pulse pattern, and wherein the measurement unit is configured to measure a parameter related to the pulse pattern.

According to an embodiment, the controller is configured to control the variable impedance in response to the pulse pattern deviating from a predefined pulse pattern.

According to an embodiment, the variable impedance comprises a resistor and a capacitor, the variable impedance comprises a resistor and an inductor, the variable impedance comprises an inductor and a capacitor, the variable impedance comprises a digitally tuned capacitor, the variable impedance comprises a digital potentiometer, or the variable impedance comprises a variable inductor.

According to an embodiment, the diameter of the primary coil is at least one of more than 0.5 cm, more than 10 cm, more than 15 cm, more than 20 cm, coil is more than 30 cm, or is more than 50 cm.

According to an embodiment, the area of the primary coil is more than 0.5 cm2, more than 2 cm2, more than 10 cm2, more than 100 cm2, more than 300 cm2, more than 500 cm2, or more than 800 cm2.

According to an embodiment, a method corresponding to the system for wirelessly charging an implant according to the previous aspect is provided.

### Aspect 456: NFMI

According to an aspect, a system for communication with an implantable medical device, when implanted in a body of a patient, is provided. The system comprises an internal communications unit, connected to or comprised in the implantable medical device, and an external communications unit, wherein the internal communications unit and the external communications unit are configured to communicate using near field magnetic induction, NFMI.

According to an embodiment, the internal communication unit comprises an internal NFMI receiver and an internal coil connected to the internal NFMI receiver, the internal NFMI receiver being configured to measure an induced voltage in the internal coil. The external communications unit comprises an external NFMI transmitter and an external coil connected to the external NFMI transmitter, and the external coil and the external NFMI transmitter are configured to modulate a magnetic field for sending data to the implantable medical device via the internal coil.

According to an embodiment, the external NFMI transmitter further comprises a capacitor for tuning.

According to an embodiment, the internal NFMI receiver comprises a tunable resistor and capacitor tank.

According to an embodiment, the internal communication unit comprises an internal NFMI transmitter and an internal coil connected to the internal NFMI transmitter. The external communications unit comprises an external NFMI receiver and an external coil connected to the external NFMI receiver, the external NFMI receiver being configured to measure an induced voltage in the external coil, and the internal coil and the internal NFMI transmitter are configured to modulate a magnetic field for sending data to the external communications unit via the external coil.

According to an embodiment, the internal NFMI transmitter further comprises a capacitor for tuning the internal coil and the internal NFMI transmitter.

According to an embodiment, the external NFMI receiver comprises a tunable resistor and capacitor tank for tuning the external coil and the external NFMI receiver.

According to an embodiment, the implantable medical device comprises an active portion configured to monitor, treat or perform a function of a body of a patient.

According to an embodiment, the active portion is not a pacemaker, a hearing aid or a neurostimulation implant.

According to an embodiment, the internal communications unit is adapted to be implanted at a tissue depth of at least 8 cm or at least 15 cm.

According to an embodiment, the internal communications unit is adapted to be implanted in an abdomen of a patient.

According to an embodiment, the external communications unit is configured to communicate with another external device.

According to an embodiment, the internal communications unit is configured to encrypt data before transmitting it to the external communications unit.

According to an embodiment, the external communications unit is configured to relay the encrypted data to the another external device without decrypting it.

### Aspect 459: Resonant Circuit

According to an aspect, an implantable medical device adapted to receive wirelessly transmitted energy is provided, the implantable medical device comprises an energy consuming part, and a first energy receiving unit, the first energy receiving unit comprising a first coil configured for receiving wirelessly transferred energy, and a first impedance unit electrically connected to the first coil, the receiving unit being configured to transfer the received energy to the energy consuming part. The implantable medical device further comprises a second energy receiving unit, the second energy receiving unit comprising a second coil configured for receiving wirelessly transferred energy and a second impedance unit electrically connected to the second coil, the receiving unit being configured to transfer the received energy to the energy consuming part. The implantable medical device further comprises a measurement unit configured to measure a parameter related to energy transfer, and a controller configured to control the subcutaneously received energy based on the parameter by controlling the first or the second impedance unit.

According to an embodiment, the first energy receiving unit has a first resonant frequency based on the inductance of the first coil and the impedance of the first impedance unit, and the second energy receiving unit has a second resonant frequency based on the inductance of the second coil and the impedance of second impedance unit.

According to an embodiment, the first receiving unit has a resonant frequency different from the resonant frequency of the second receiving unit.

According to an embodiment, the first and second impedance units are connected in parallel to the respective coil.

According to an aspect, an implantable medical device adapted to receive wirelessly transmitted energy, the implantable medical device comprises an energy consuming part, and a receiving unit configured for receiving wirelessly transferred energy and transferring the received energy to the energy consuming part, the receiving unit comprising a first coil portion and a second coil portion, and a first impedance unit and a second impedance unit, wherein the first impedance unit is connected to the first coil portion and the second impedance unit is connected to the second coil portion. The implantable medical device further comprises a measurement unit configured to measure a parameter related to energy transfer, and a controller configured to control the subcutaneously received energy based on the parameter by controlling the first or the second impedance unit.

According to an embodiment, the first coil portion and the second coil portion are at least one of portions of the same coil, or portions or different coils connected in series.

According to an embodiment, the first coil portion and the second coil portion have the same inductance, or the first coil portion has a different inductance than the second coil portion.

According to an embodiment, the first impedance is connected in parallel to the first coil portion and the second impedance is connected in parallel to the second coil portion.

According to an embodiment, one of the first coil portion and the second coil portion are overlapping the other of the first coil portion and the second coil portion, or the first coil portion and the second coil portion are not overlapping with the other of the first coil portion and the second coil portion.

According to an embodiment, the first coil portion and the first impedance unit has a first resonance frequency, and the second coil portion and the second impedance unit has a second resonance frequency.

According to an embodiment, the first resonance frequency is different from the second resonance frequency.

According to an embodiment, the first or second impedance unit is a capacitor.

According to an embodiment, the first impedance unit and the second impedance unit have different impedances.

According to an aspect, an implantable medical device adapted to receive wirelessly transmitted energy, the implantable medical device comprises an energy consuming part, and a first receiving unit comprising a first coil configured for receiving wirelessly transferred energy and transferring the received energy to the energy consuming part, and a first impedance electrically connected to the coil. The implantable medical device further comprises a second receiving unit comprising a second coil portion and a third coil portion configured for receiving wirelessly transferred energy and transferring the received energy to the energy consuming part, and a second impedance unit and a third impedance unit, wherein the second impedance unit is connected to the second coil portion and the third impedance unit is connected to the third coil portion. The implantable medical device further comprises a measurement unit configured to measure a parameter related to energy transfer, and a controller configured to control the subcutaneously received energy based on the parameter by controlling the first, the second or the third impedance unit.

### BRIEF DESCRIPTION OF THE DRAWINGS

Figures are to be considered schematic rather than photorealistic illustrations. Intermittent or dashed borders for flowchart borders may indicate optional steps.

FIG. 1A illustrates a system comprising an implant, further illustrated in FIG. 1B, and an external device, further illustrated in FIG. 1C, all according to the aspect 244SE.

FIG. 2A illustrates a system according to the aspect 244SE, comprising an implant and an external device, further illustrated in 2B.

FIG. 2B illustrates an external device.

FIGS. 3-7 illustrate flowcharts of methods according to embodiments of the second part of the aspect 244SE.

FIG. 8A illustrates a system comprising an implant, further illustrated in FIG. 8B, and an external device, further illustrated in FIG. 8C, all according to aspect 245SE.

FIG. 9 illustrates a system according to aspect 245SE comprising an implant in connection with an external device.

FIG. 10 illustrates a system according to aspect 245SE comprising an implant in connection with an external device and a second external device.

FIG. 11 illustrates a system according to aspect 245SE comprising an implant in connection with an external device wherein the external device is in connection with a second external device.

FIGS. 12-17 illustrate flowcharts of methods according to embodiments of the first part of aspect 245SE.

FIG. 18A illustrates a system comprising an implant, further illustrated in FIG. 18B, and an external device, further illustrated in FIG. 18C, all according to aspect 246SE.

FIGS. 19-21 illustrate systems according to aspect 246SE comprising an implant in connection with an external device wherein either the implant or the external device is in connection with a second external device, third external device, or other external devices.

FIGS. 22-29 illustrate flowcharts of methods according to embodiments of the first part of aspect 246SE.

FIG. 30A illustrates a system comprising an implant, further illustrated in FIG. 30B, and an external device, further illustrated in FIG. 30C, all according to aspect 247SE.

FIG. 31 illustrates a system according to aspect 247SE comprising an implant in connection with an external device.

FIGS. 32-35 illustrate flowcharts of methods according to embodiments of the second part of aspect 247SE.

FIG. 36A illustrates a system comprising an implant, further illustrated in FIG. 36B, and an external device, further illustrated in FIG. 36C, all according to aspect 248SE.

FIGS. 37-38 illustrate systems according to aspect 248SE comprising an implant in connection with an external device.

FIG. 39 illustrates a flowchart of methods according to embodiments of the first part of aspect 248SE.

FIGS. 40-48 illustrate flowcharts of methods according to embodiments of the first part of aspect 249SE.

FIG. 49A illustrates a system comprising an implant, further illustrated in FIG. 49B, and an external device, further illustrated in FIG. 49C, all according to aspect 249SE.

FIG. 50 illustrates a system according to aspect 249SE comprising an implant, an external device, and a second external device, all connected with each other.

FIG. 51A illustrates a system comprising an implant, further illustrated in FIG. 51B, and an external device, further illustrated in FIG. 51C, all according to aspect 250SE.

FIG. 52 illustrates a system according to aspect 250SE comprising an implant in connection with an external device wherein the external device is in connection with a second external device wherein the second is in connection with a third external device.

FIGS. 53-55 illustrate flowcharts of methods according to embodiments of the second part of aspect 250SE.

FIGS. 56-60 illustrate flowcharts of methods according to embodiments of the first part of aspect 251SE.

FIG. 61A illustrates a system comprising an implant, further illustrated in FIG. 61B, and an external device, further illustrated in FIG. 61C, all according to aspect 251SE.

FIG. 62 illustrates a system according to aspect 251SE comprising an implant in connection with an external device and a second external device.

FIG. 63A illustrates a system with the first and the second point transmitting such that destructive interference occurs at the at least one point located at different distances from the first and second points.

FIG. 63B illustrates a system with the first and the second point transmitting such that constructive interference occurs at the at least one point located at different distances from the first and second points.

FIG. 64 illustrates a system with the first and the second point being equidistant to the at least one point and how constructive and destructive interference may be achieved by phase shifting the transmission from the second point relative to the transmission from the first point.

FIG. 65-67 illustrate flowcharts of methods according to embodiments of the first part of aspect 252SE.

FIG. 68A illustrates a system comprising an implant, further illustrated in FIG. 68B, and an external device, further illustrated in FIG. 68C, all according to aspect 252SE.

FIG. 69 illustrates a system according to aspect 252SE comprising an implant in connection with an external device.

FIG. 70 illustrates a system according to aspect 252SE comprising an implant, an external device, a second external device and a third external device including connections between them.

FIG. 71 illustrates a flowchart of methods according to embodiments of the second part of aspect 253SE.

FIG. 72A illustrates a system comprising an implant, further illustrated in FIG. 72B, and an external device, further illustrated in FIG. 72C, all according to aspect 253SE.

FIG. 73 illustrates a system according to aspect 253SE comprising an implant in connection with an external device wherein the external device is in connection with a second external device.

FIGS. 74-82 illustrate flowcharts of methods according to embodiments of the first part of aspect 254SE.

FIG. 83A illustrates a system comprising an implant, further illustrated in FIG. 83B, and an external device, further illustrated in FIG. 83C, all according to aspect 254SE.

FIG. 84 illustrates a system according to aspect 254SE comprising an implant in connection with an external device wherein the external device is in connection with a second external device.

FIG. 85A illustrates an implant according to aspect 255SE being implanted in connection with the vascular system of a patient.

FIG. 85B illustrates an implant according to aspect 255SE being implanted in connection with the intestinal system of a patient.

FIG. 85C-D illustrates an implant according to aspect 255SE being implanted in connection with the heart of a patient.

FIG. 85E illustrates an implant according to aspect 255SE being implanted in connection with the pulmonary system of a patient.

FIG. 85F illustrates an implant according to aspect 255SE being implanted in connection with the urinary system of a patient.

FIG. 85G illustrates an audio implant according to aspect 255SE being implanted in a patient.

FIG. 85H illustrates an audio implant according to aspect 255SE being ingested by a patient.

FIG. 86A illustrates a system comprising an implant, further illustrated in FIG. 86B, and an external device, further illustrated in FIG. 86C, all according to aspect 256SE.

FIG. 87 illustrates a system according to aspect 256SE comprising an implant in connection with an external device.

FIGS. 88-89 illustrate flowcharts of methods according to embodiments of the first part of aspect 256SE.

FIG. 90A illustrates a system comprising an implant, further illustrated in FIG. 90B, and an external device, further illustrated in FIG. 90C, all according to aspect 257SE.

FIG. 91 illustrates a system according to aspect 257SE comprising an implant in connection with an external device and a sensation generator within the body of a patient.

FIG. 92 illustrates a system according to aspect 257SE comprising an implant in connection with an external device and a sensation generator located on or outside of the body of a patient.

FIG. 93 illustrates a flowchart of methods according to embodiments of the first part of aspect 257SE.

FIG. 94 illustrates a system according to embodiments of the ninth part of aspect 255SE.

FIG. 95 illustrates a system according to the fifth part of aspect 251SE comprising an external device in connection with an implant.

FIG. 96 illustrates a system according to embodiments of the fifth part of aspect 251SE.

FIG. 97 illustrates a system according to aspect 307SE.

FIG. 98 illustrates a method according to aspect 307SE.

FIG. 99 illustrates a system according to aspect 308SE.

FIG. 100 illustrates a method according to aspect 308SE.

FIGS. 101A, 101B and 101C illustrate a system according to aspect 309SE.

FIG. 102 illustrates a method according to aspect 309SE.

FIG. 103 illustrates a system according to aspect 31OSE.

FIG. 104 illustrates a method according to aspect 310SE.

FIGS. 105A, 105B and 105C illustrate a system according to aspect 311SE.

FIG. 106 illustrates a method according to aspect 311 SE.

FIG. 107 illustrates a method according to aspect 311 SE.

FIGS. 108A and 108B illustrate a system according to aspect 312SE.

FIG. 109 illustrates a method according to aspect 312SE.

FIG. 110 illustrates a system according to aspect 313SE.

FIG. 111 illustrates a method according to aspect 313SE.

FIG. 112 illustrates a method according to aspect 313SE.

FIGS. 113A, 113B, 113B′ and 113C generally illustrates a system for communication with an implanted medical device.

FIGS. 114A and 114B illustrates a method according to aspect 314SE.

FIG. 115 illustrates a system according to aspect 315SE.

FIG. 116 illustrates a method according to aspect 315SE.

FIG. 117 illustrates a system according to aspect 316SE.

FIG. 118 illustrates a method according to aspect 316SE.

FIG. 119A-119H shows embodiments and describes various functions of an implantable controller for controlling the implantable medical device/implant and a system for communication between different external devices.

FIG. 120 shows an elevated perspective view from the left of a housing unit.

FIG. 121 shows a plain view from the left of a housing unit.

FIG. 122 shows an elevated perspective view from the left of a housing unit.

FIG. 123 shows a plain view from the left of a housing unit.

FIG. 124 shows a system overview of an external device comprising a housing unit and a display device in wireless communication with an implanted medical device.

FIG. 125 shows a cross-sectional view of an implantable energized medical device for powering an implantable medical device with hydraulic force.

FIG. 126 shows an exploded cross-sectional view of an implantable energized medical device for powering an implantable medical device with hydraulic force.

FIG. 127A shows a detailed cross-sectional view of a first unit of an implantable energized medical device for powering an implantable medical device with hydraulic force.

FIG. 127B shows a detailed cross-sectional view of a first unit of an implantable energized medical device for powering an implantable medical device with hydraulic force.

FIG. 127C shows a detailed cross-sectional view of a first unit of an implantable energized medical device for powering an implantable medical device with hydraulic force.

FIG. 127D shows a detailed cross-sectional view of a first unit of an implantable energized medical device for powering an implantable medical device with hydraulic force.

FIGS. 128A-128B, 129A-129B, 130A-130B show alternative embodiments of connecting portions for an implantable energized medical device.

FIG. 131 shows, schematically, a kit of components forming an implantable energized medical device.

FIG. 132 shows a detailed cross-sectional view of an embodiment of an implantable energized medical device for powering an implantable medical device.

FIG. 133 shows a perspective elevated view from the right of an embodiment of an implantable energized medical device for powering an implantable medical device.

FIG. 134 shows a perspective elevated view from the right of a portion of an embodiment of an implantable energized medical device for powering an implantable medical device.

FIG. 135 shows a perspective elevated view from the right of a portion of an embodiment of an implantable energized medical device for powering an implantable medical device.

FIG. 136A shows a cross-sectional plain side view of an embodiment of an implantable energized medical device for powering an implantable medical device.

FIG. 136B shows a cross-sectional plain side view of an embodiment of an implantable energized medical device for powering an implantable medical device.

FIG. 136C shows a cross-sectional plain side view of an embodiment of an implantable energized medical device for powering an implantable medical device.

FIG. 136D shows a cross-sectional plain side view of an embodiment of an implantable energized medical device for powering an implantable medical device.

FIGS. 137A-K, 137M, 137N, 137P and 137Q show perspective elevated views from the right of embodiments of an implantable energized medical device for powering an implantable medical device.

FIG. 137R shows a perspective elevated view from the right of an embodiment of an implantable energized medical device for powering an implantable medical device.

FIG. 137S shows a plain top view of an embodiment of an implantable energized medical device for powering an implantable medical device.

FIGS. 137T and 137U show, schematically, plain top views of two embodiments of implantable energized medical devices for powering implantable medical devices.

FIGS. 137X-137Z illustrate three stages of insertion and fixation of an embodiment of an implantable energized medical device for powering an implantable medical device.

FIG. 137AA shows a detailed cross-sectional view of an embodiment of an implantable energized medical device for powering an implantable medical device.

FIG. 137BB shows, schematically, a portion of an implantable energized medical device for powering an implantable medical device.

FIG. 137CC shows, schematically, a portion of an implantable energized medical device for powering an implantable medical device.

FIG. 137DD shows, schematically, a portion of an implantable energized medical device for powering an implantable medical device.

FIG. 137EE shows a frontal view of a human patient in cross section when a remotely powered medical device for actively stretching a stomach wall of a patient has been implanted.

FIG. 137FF shows a frontal view of a human patient in cross section when a remotely powered medical device for affecting the flow of urine of a patient has been implanted.

FIG. 137GG shows a sectional side view of an embodiment of a hydraulic pump for an implantable medical device.

FIG. 137HH shows a sectional side view of an embodiment of a hydraulic pump for an implantable medical device.

FIG. 13711 shows a partially sectional perspective view from the left of an embodiment of a hydraulic pump for an implantable medical device.

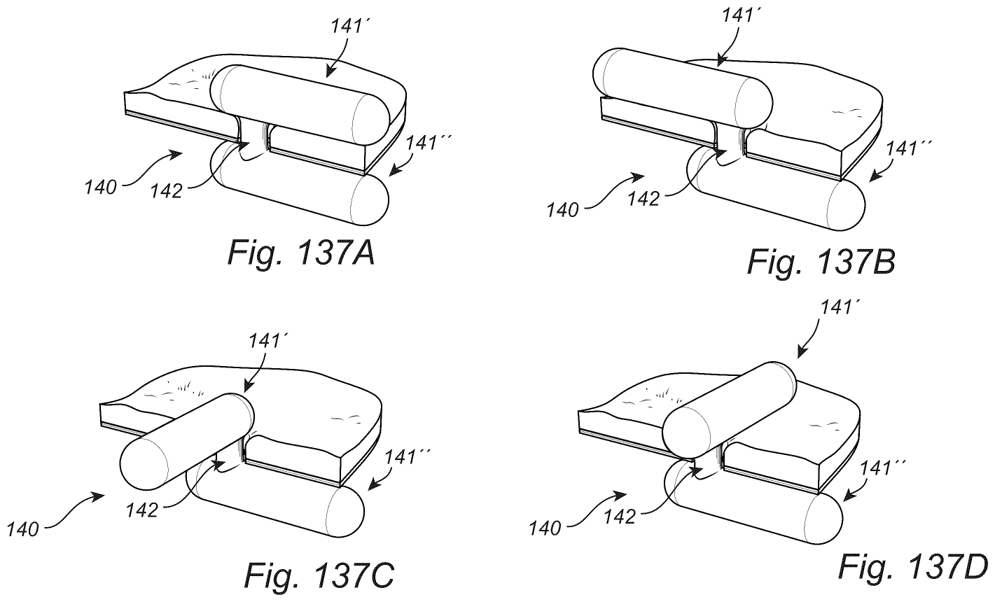

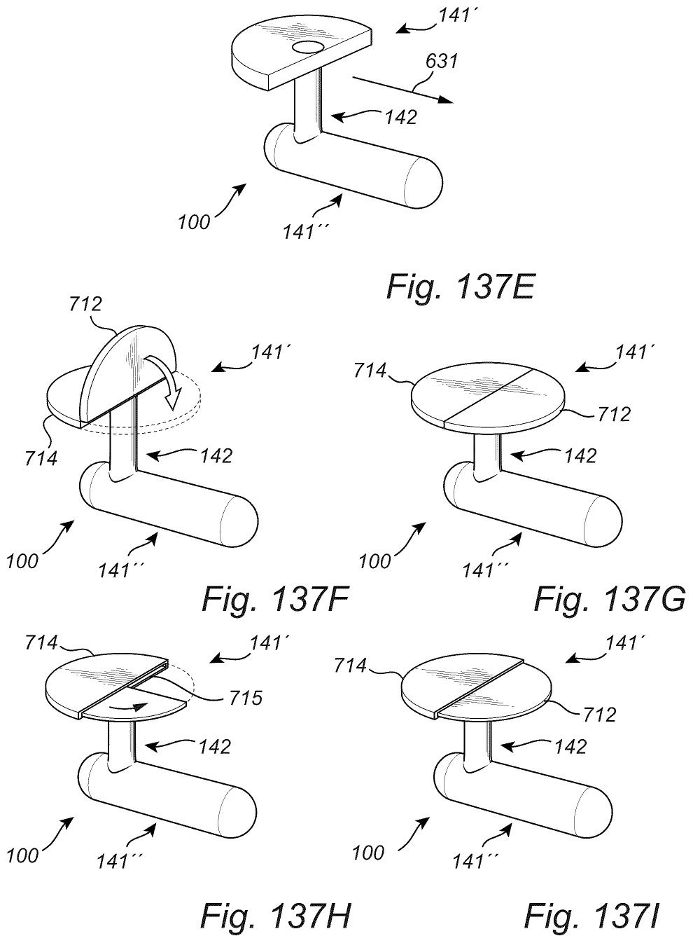

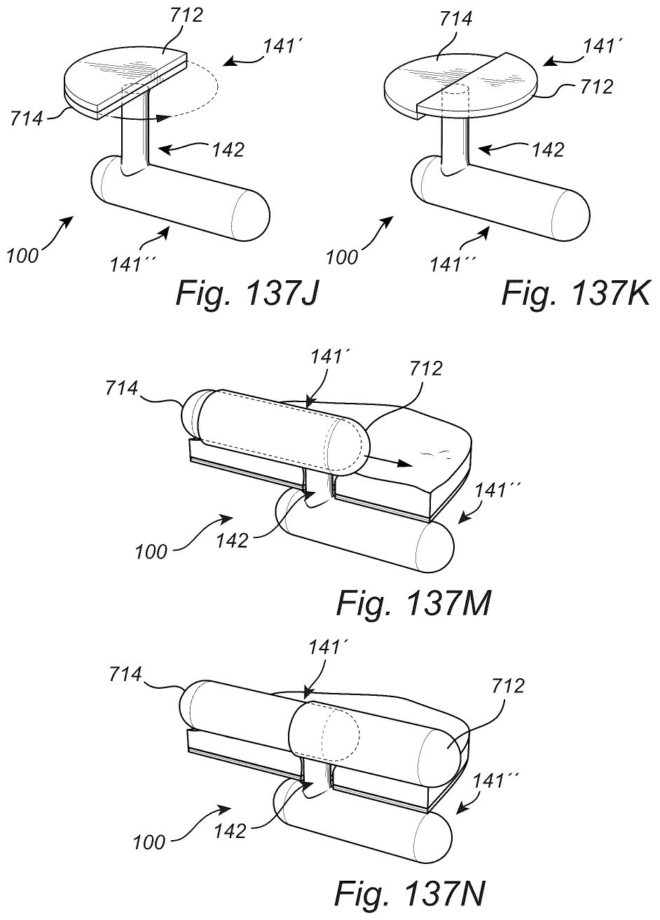

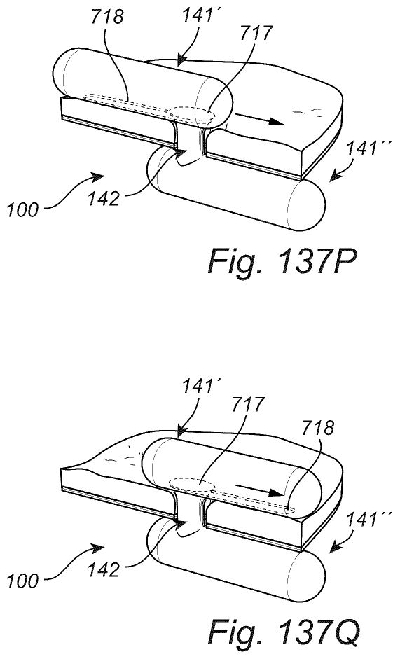

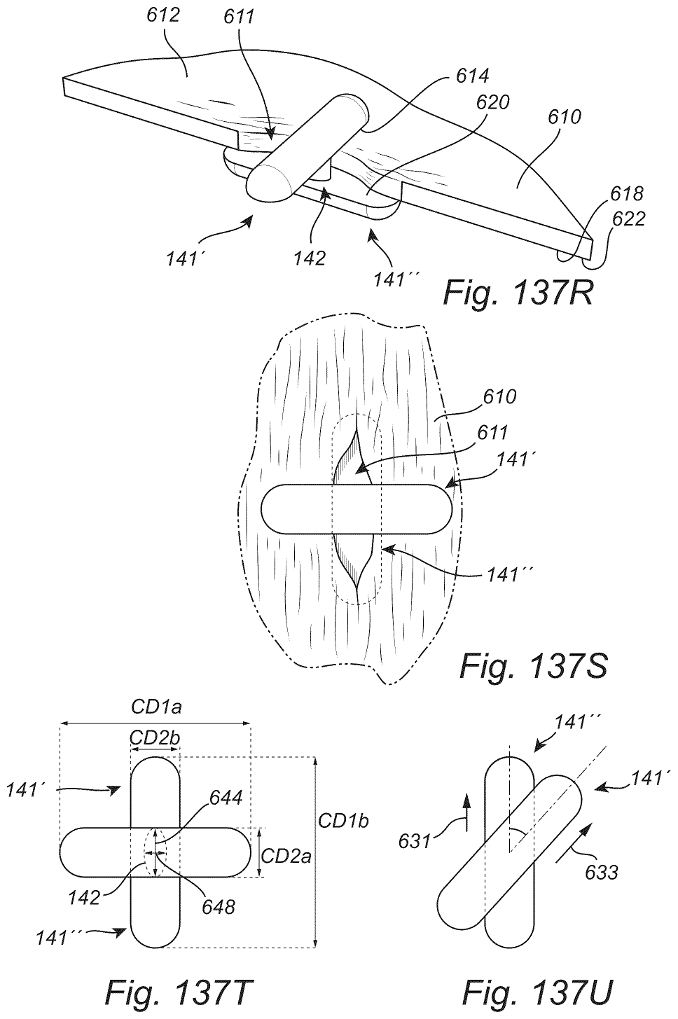

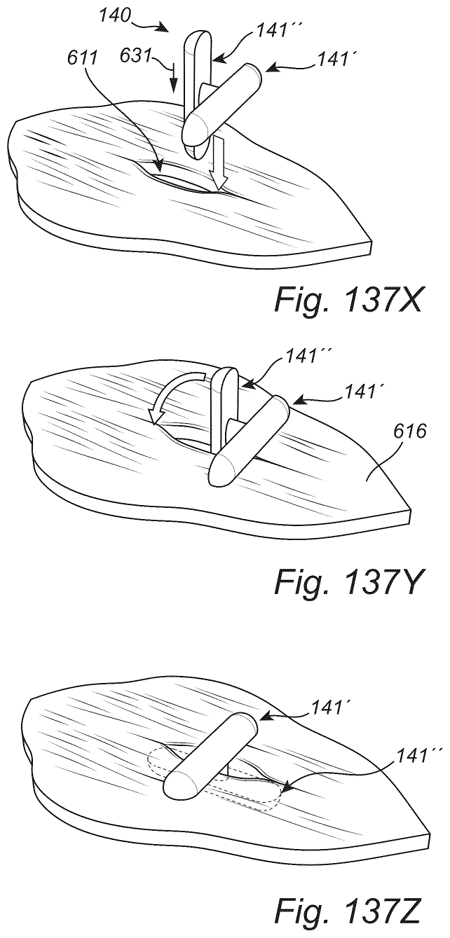

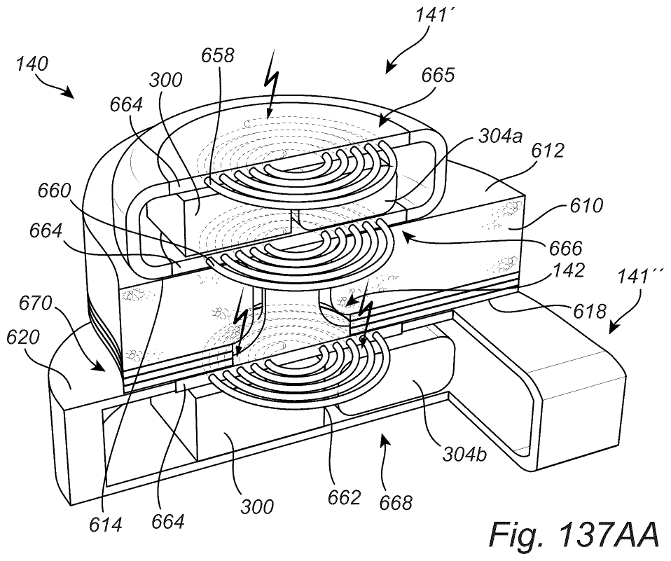

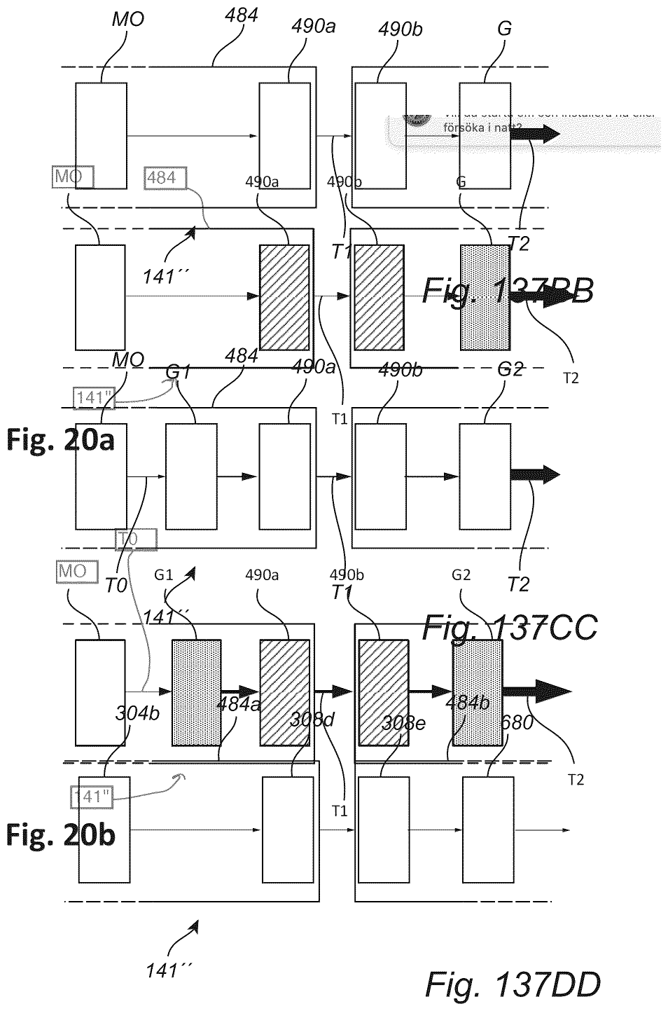

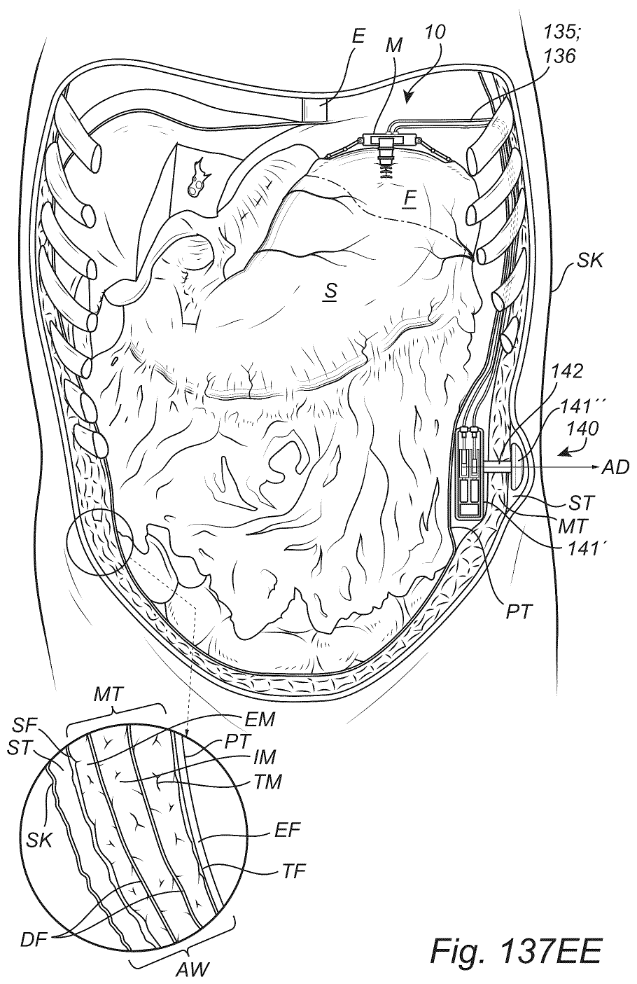

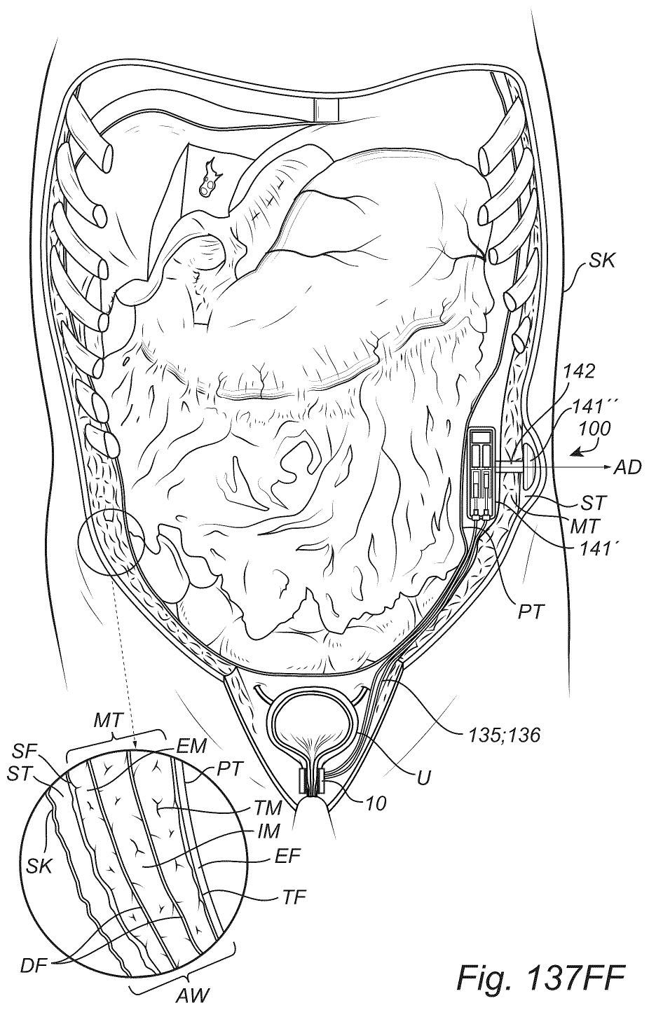

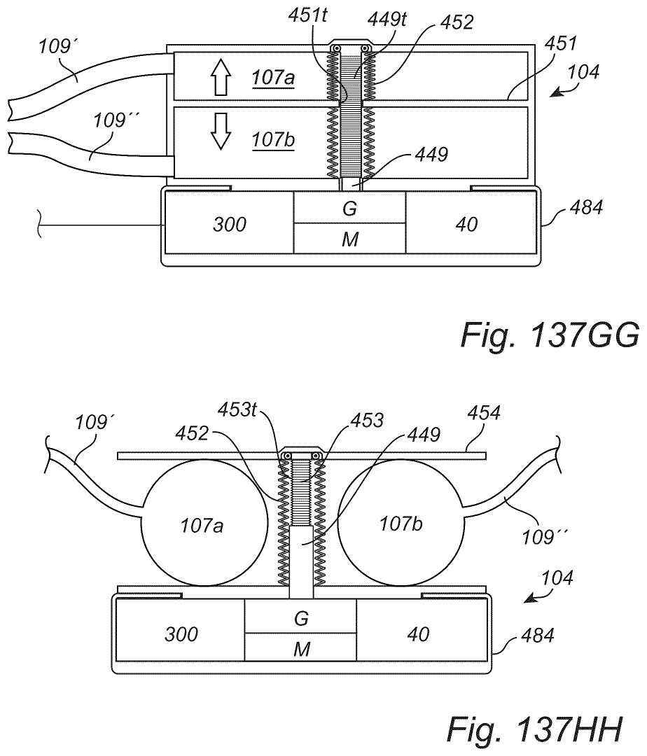

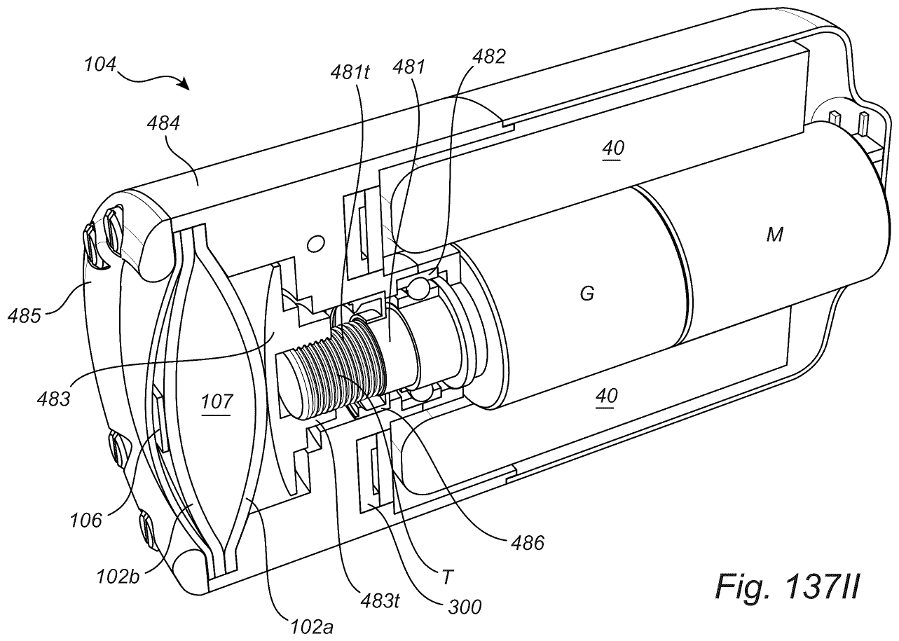

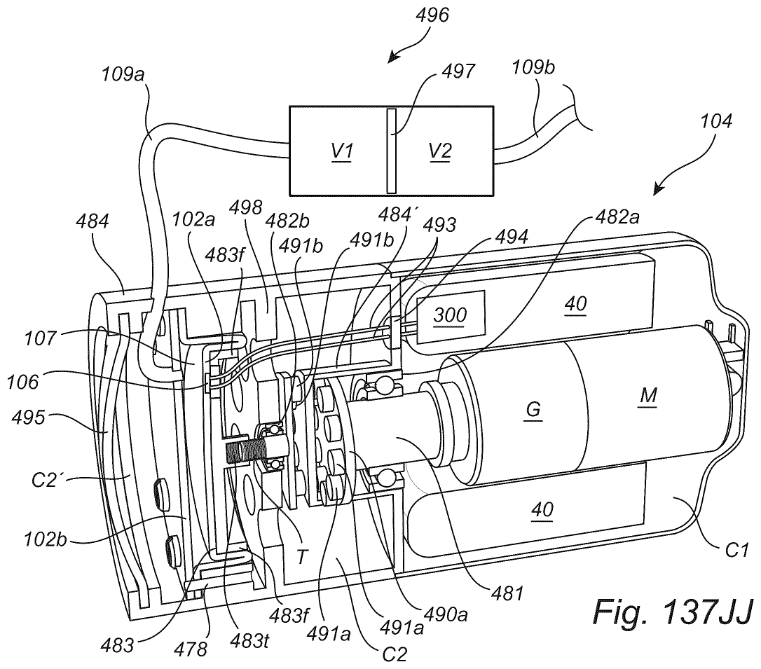

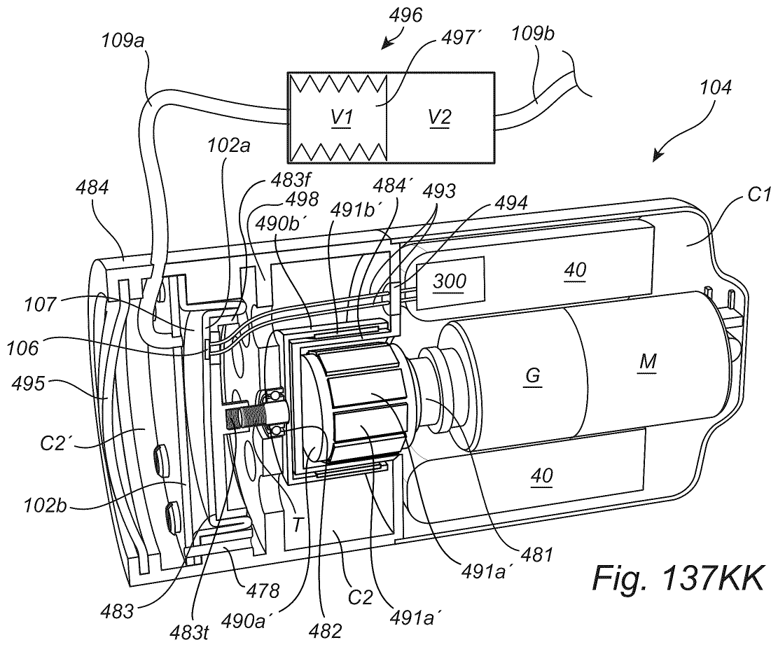

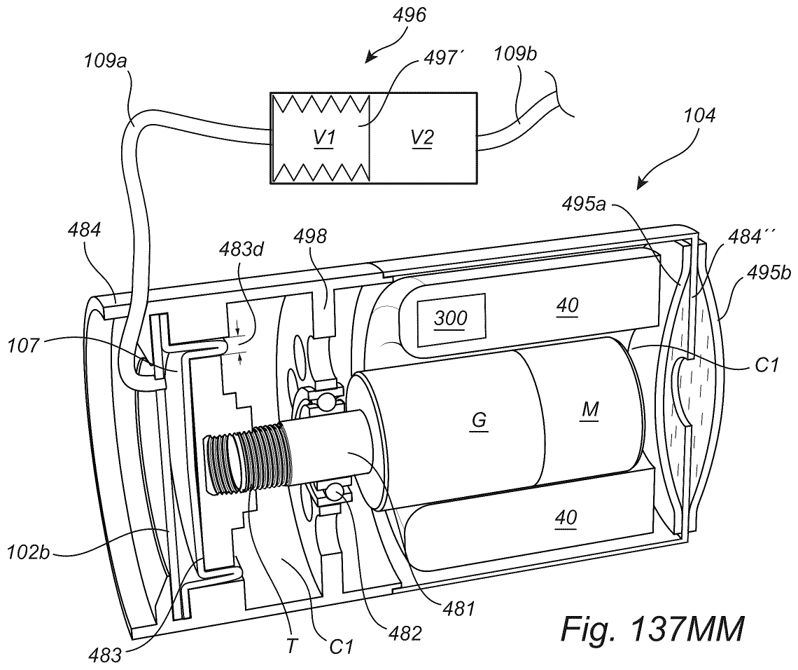

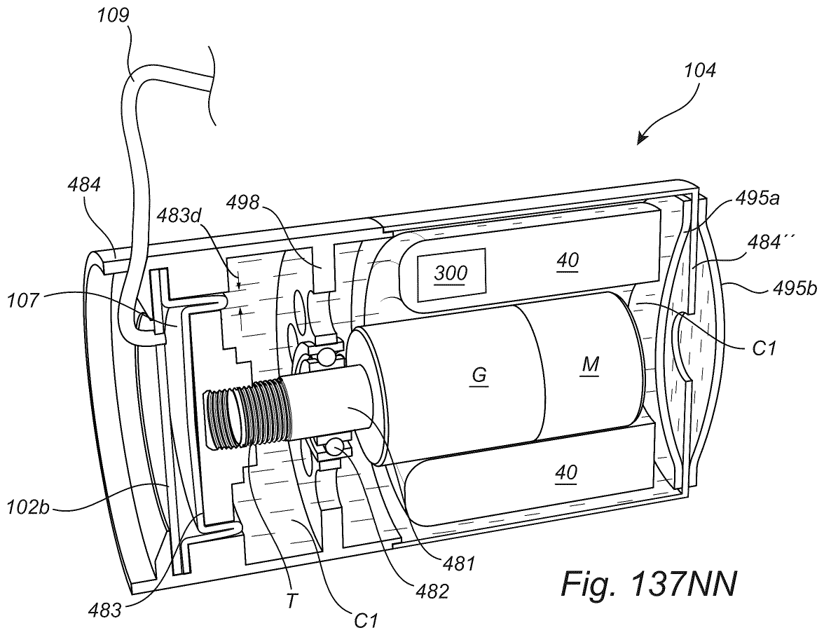

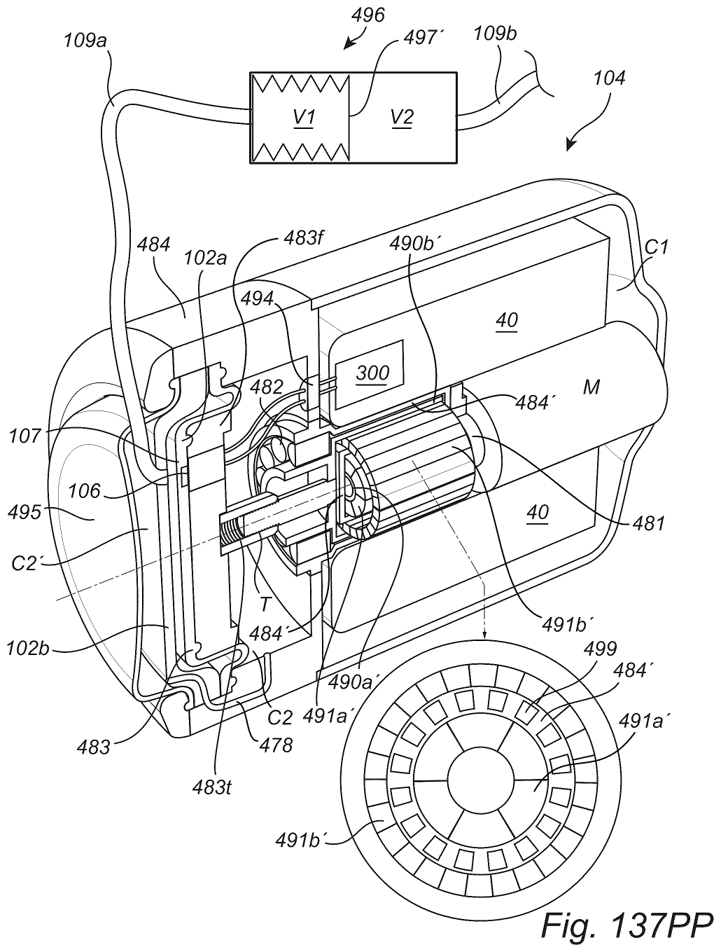

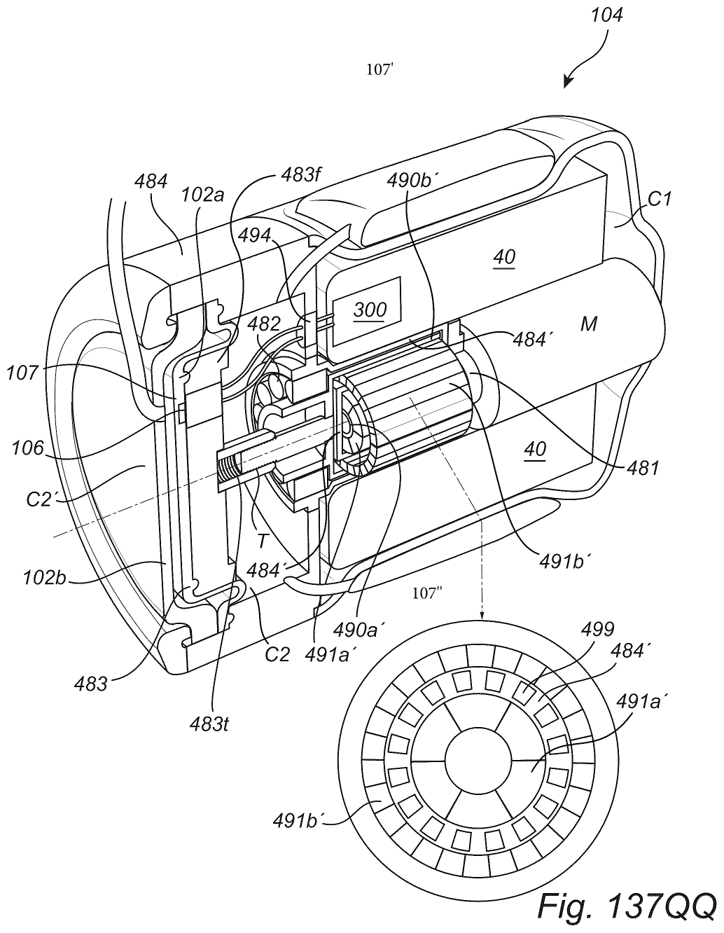

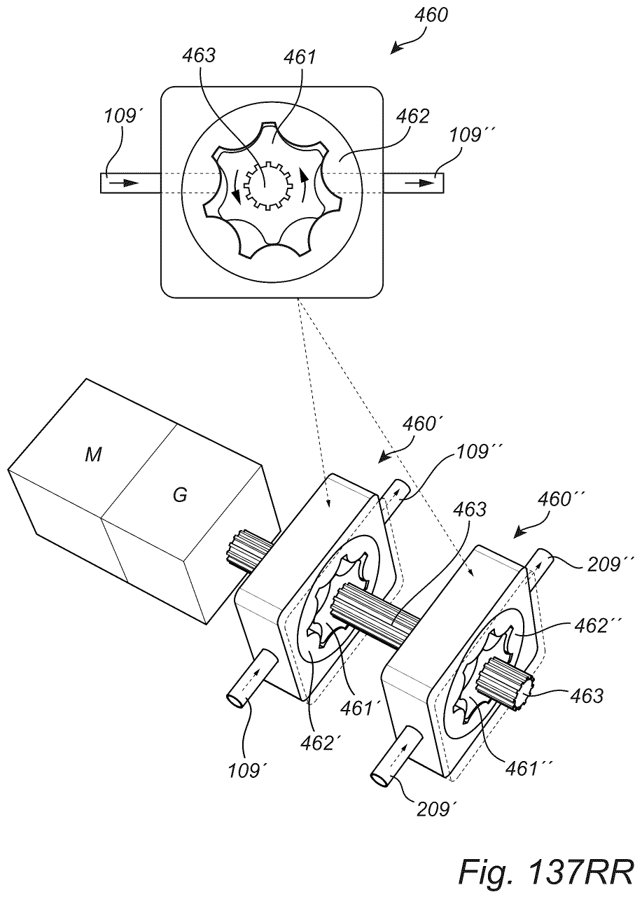

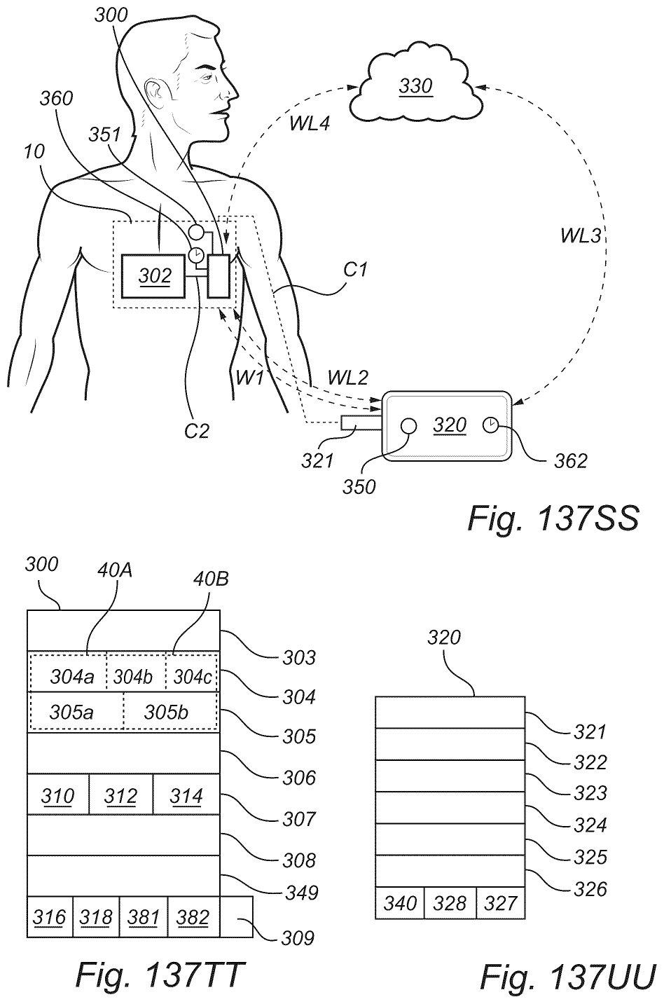

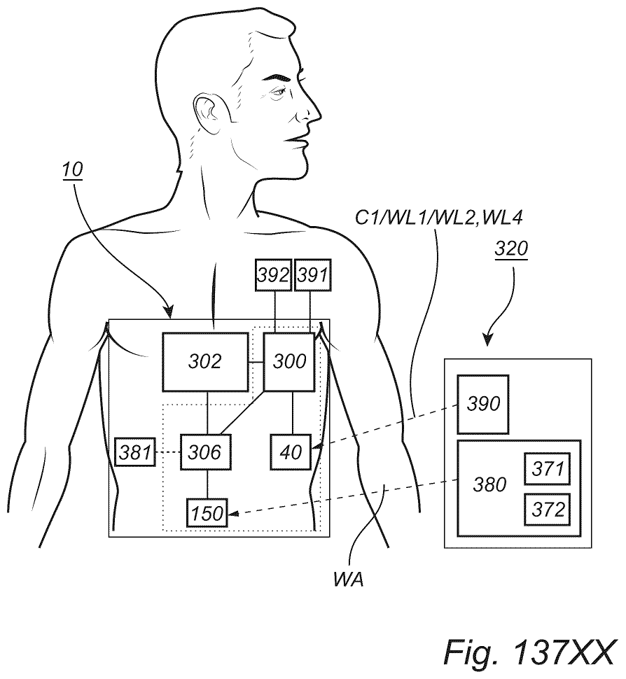

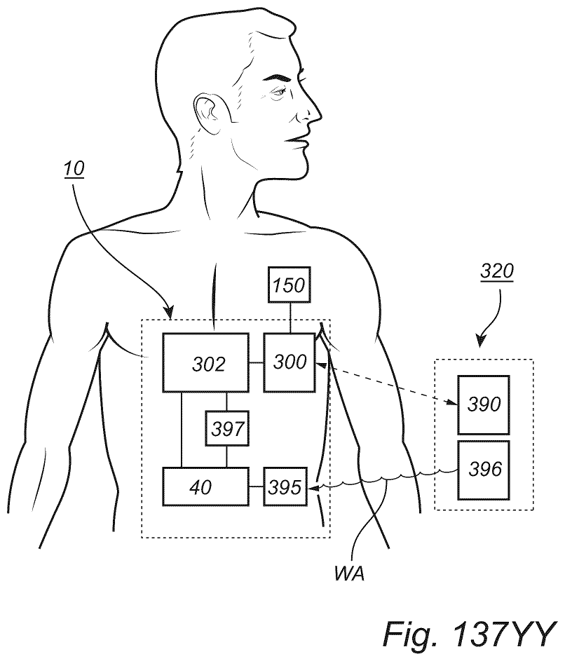

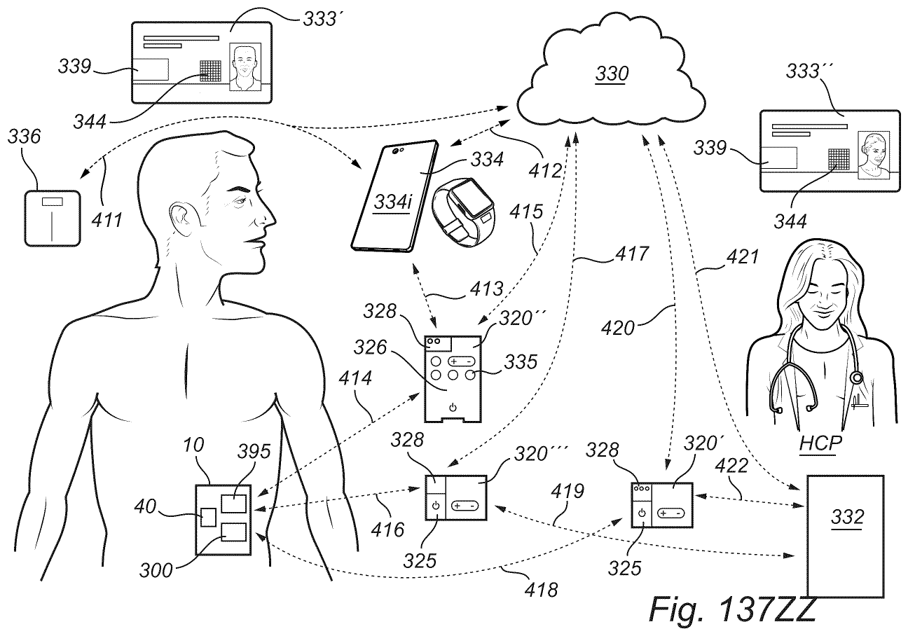

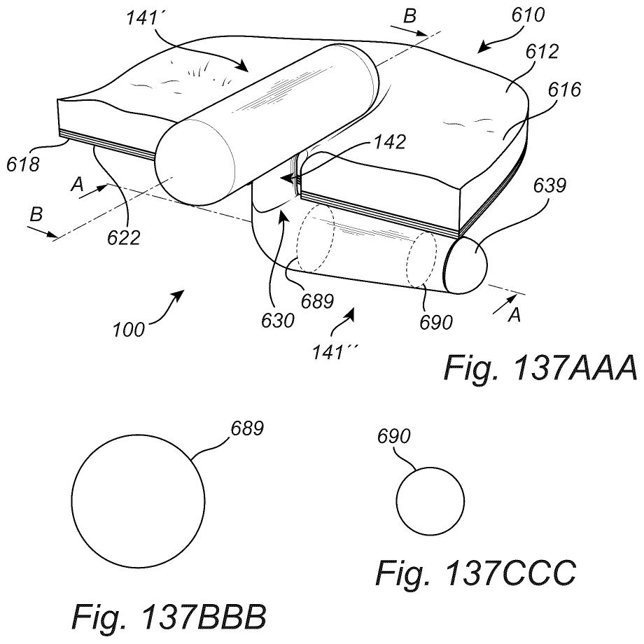

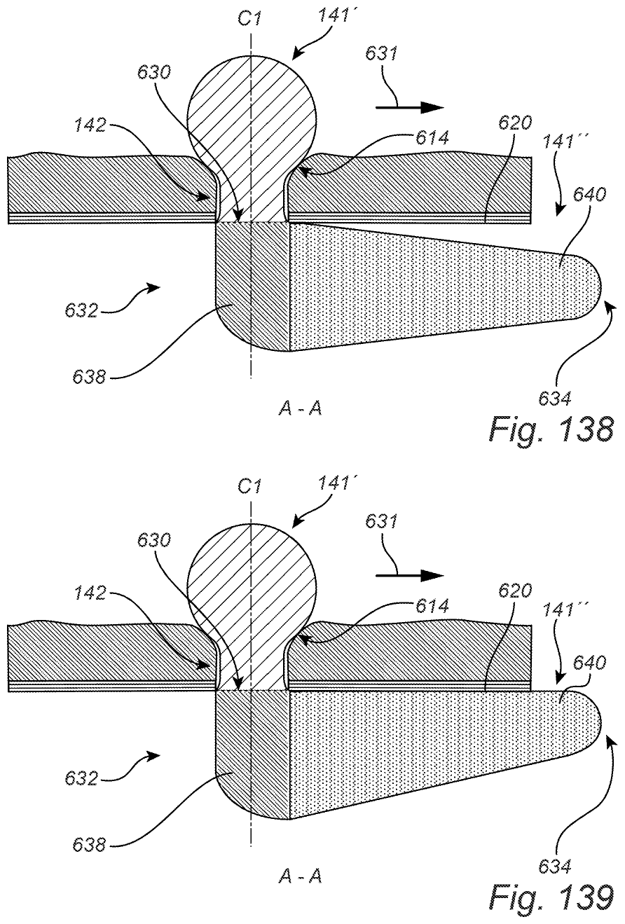

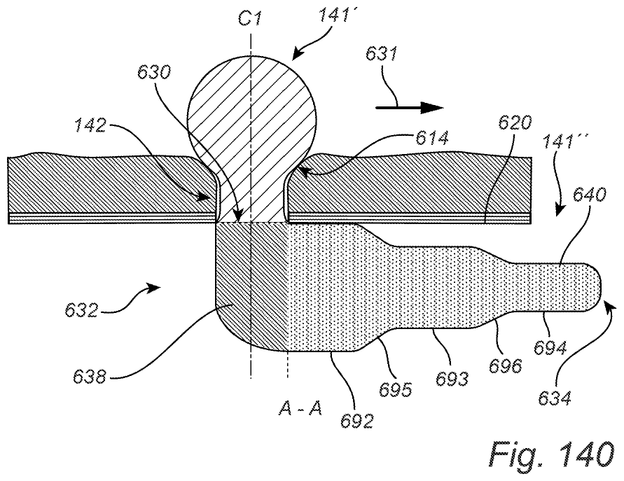

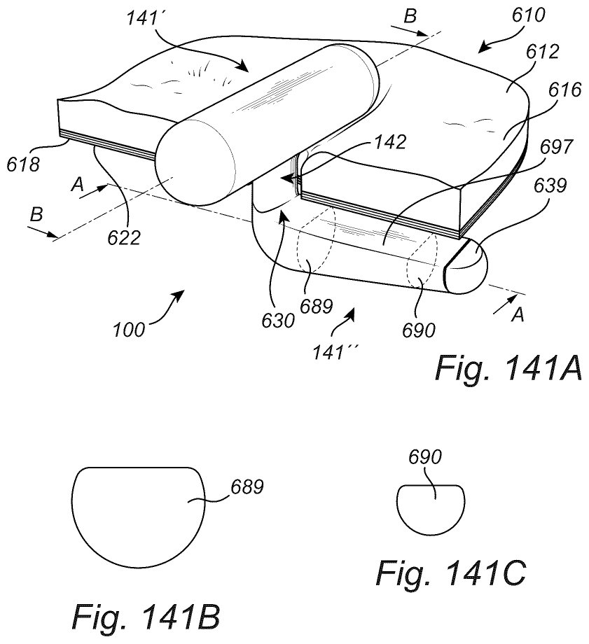

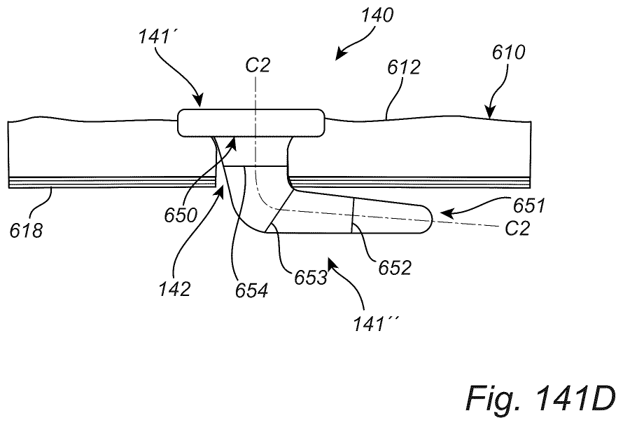

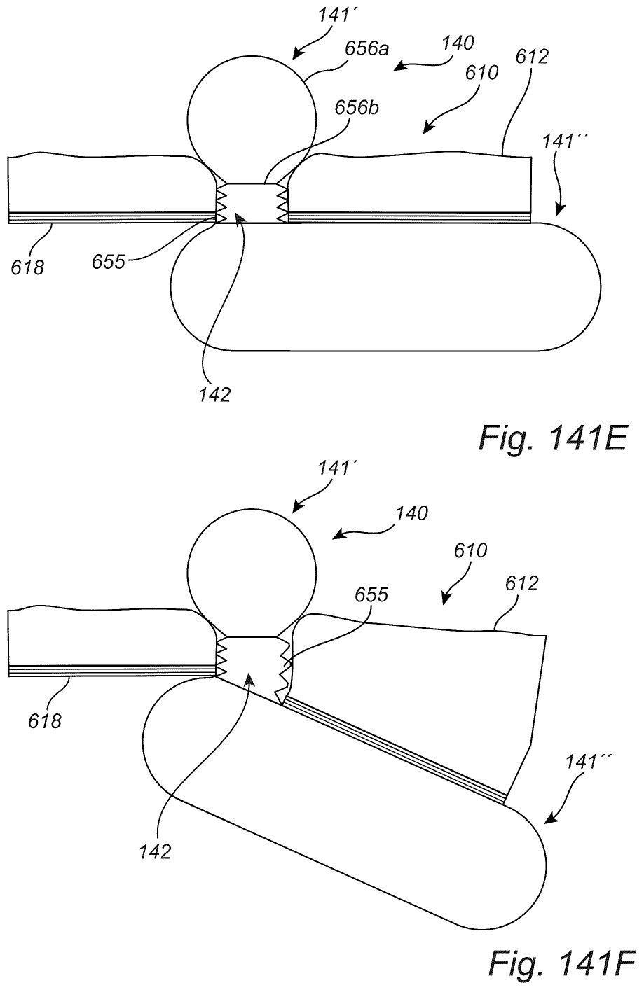

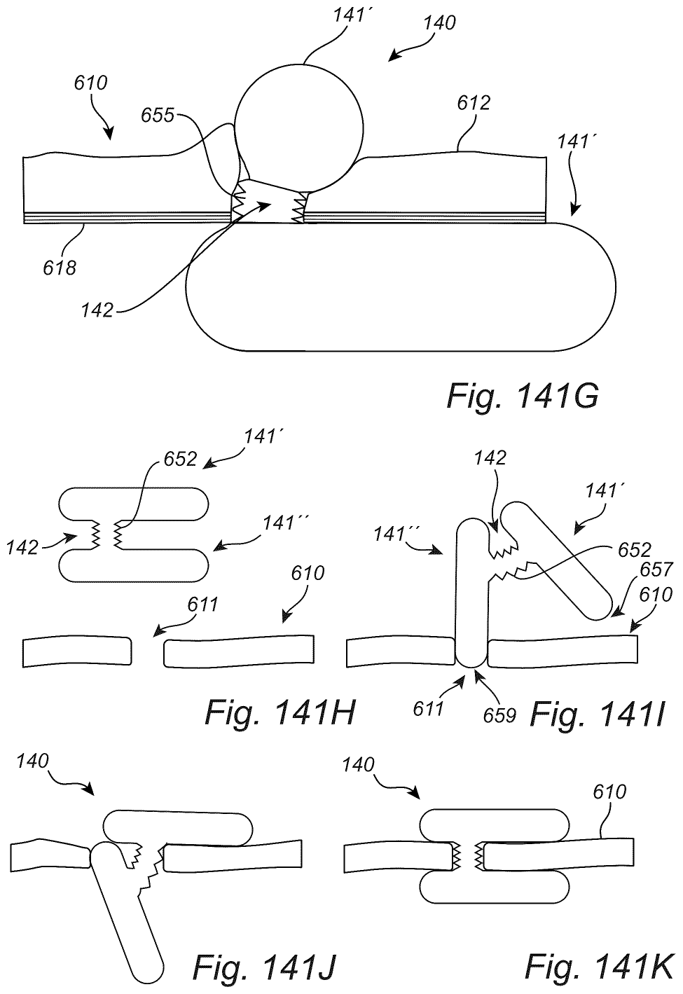

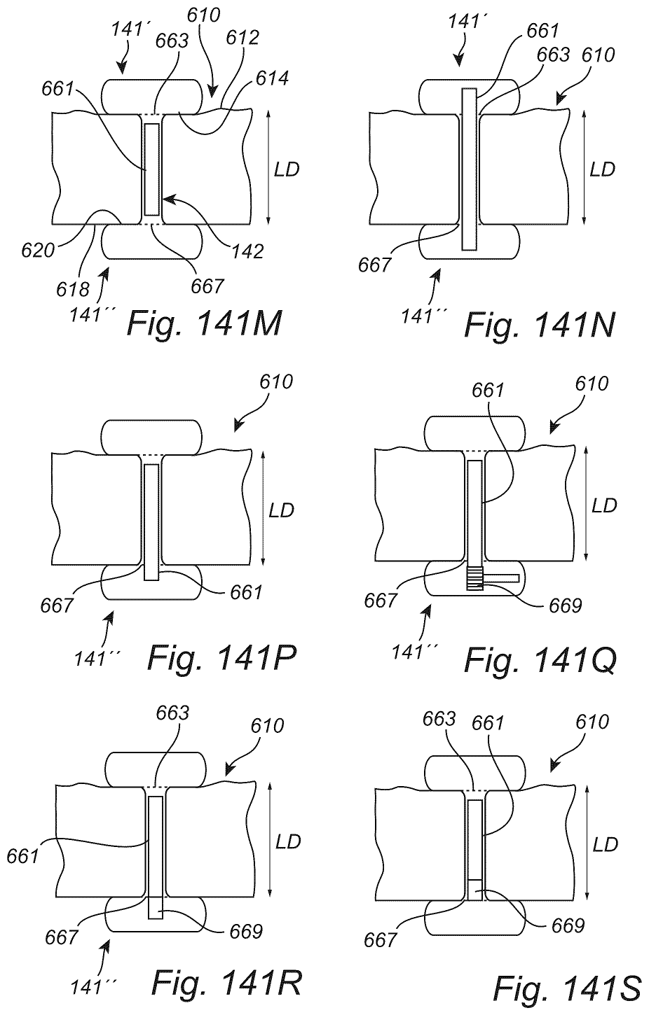

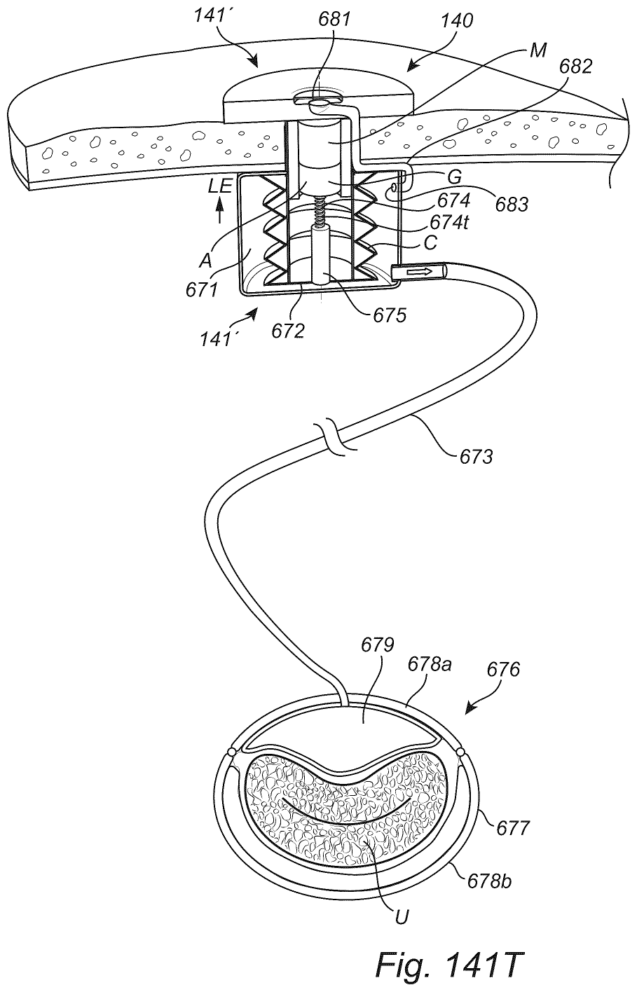

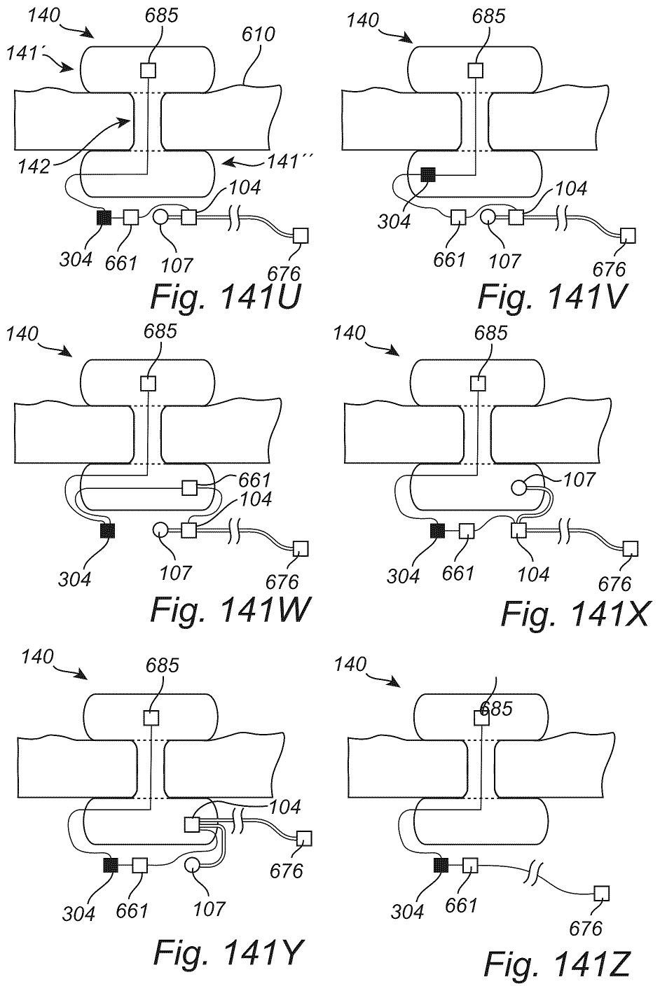

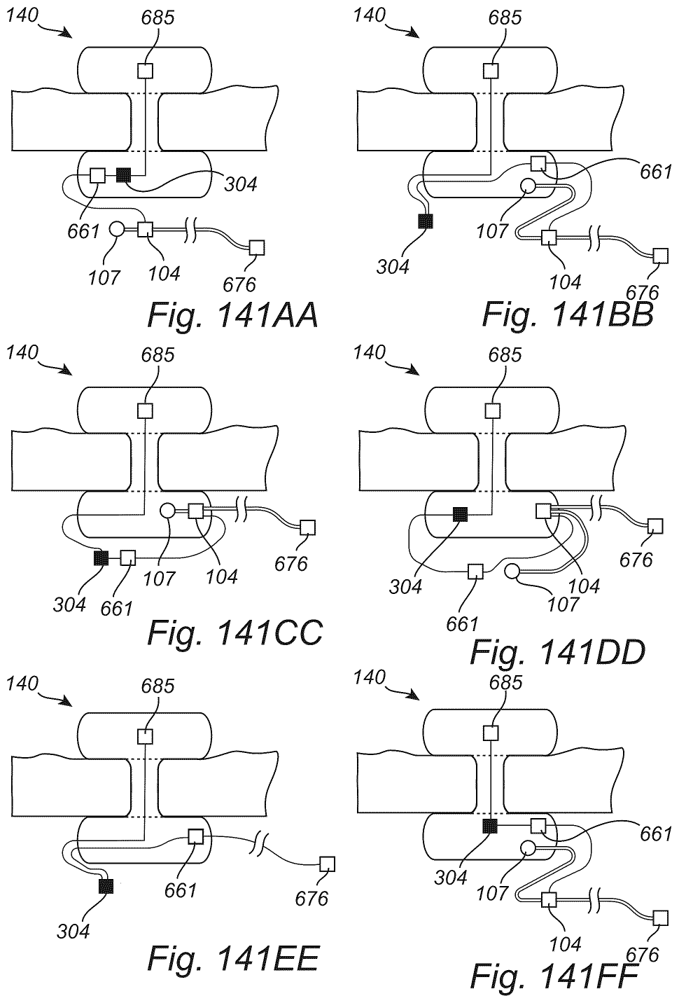

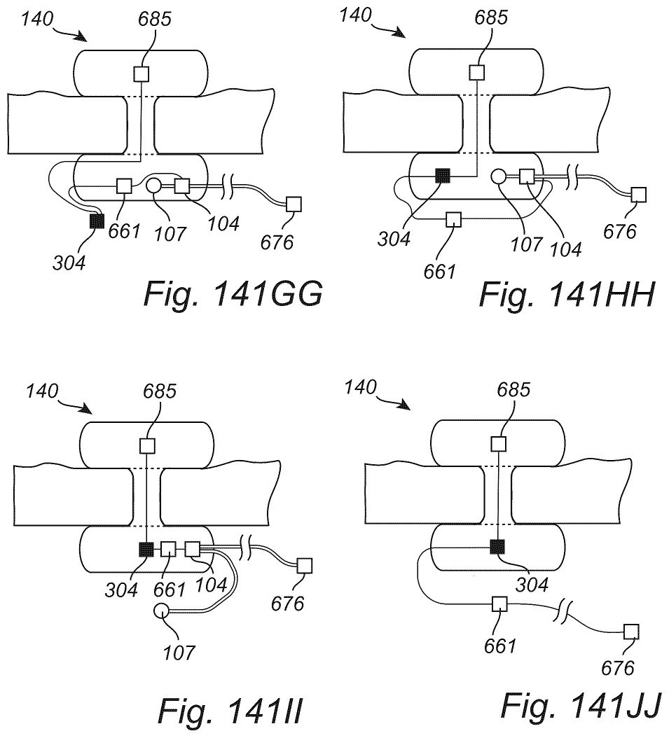

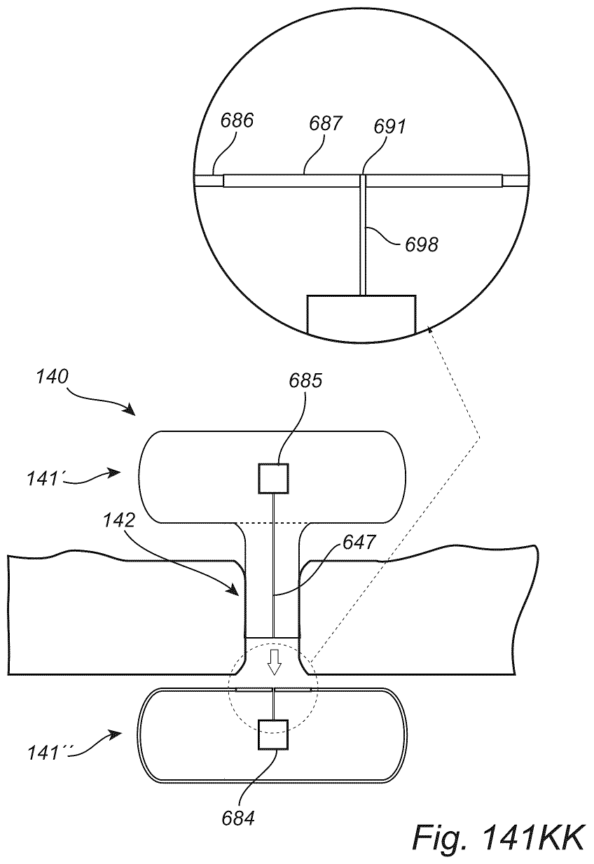

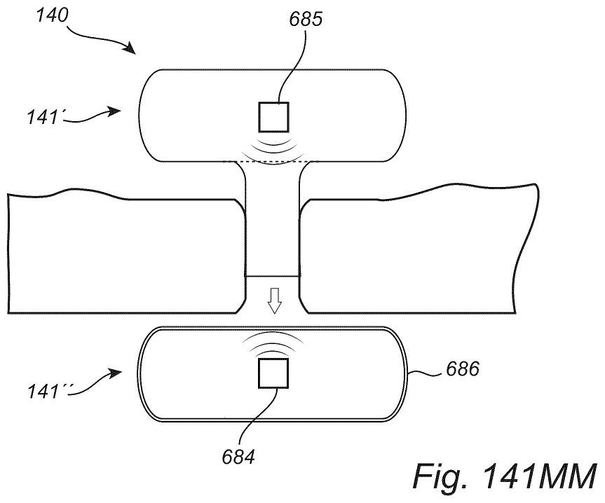

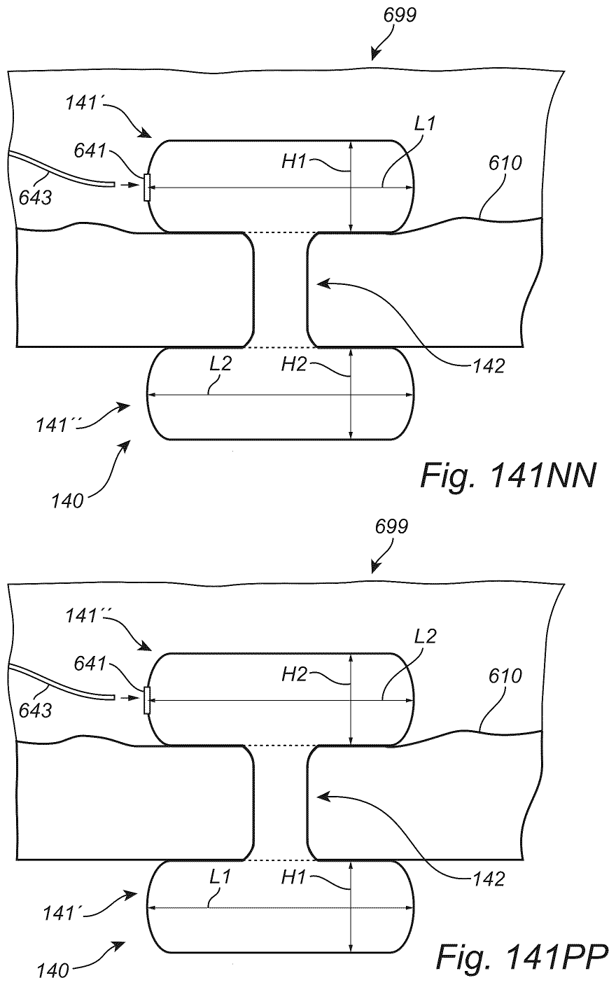

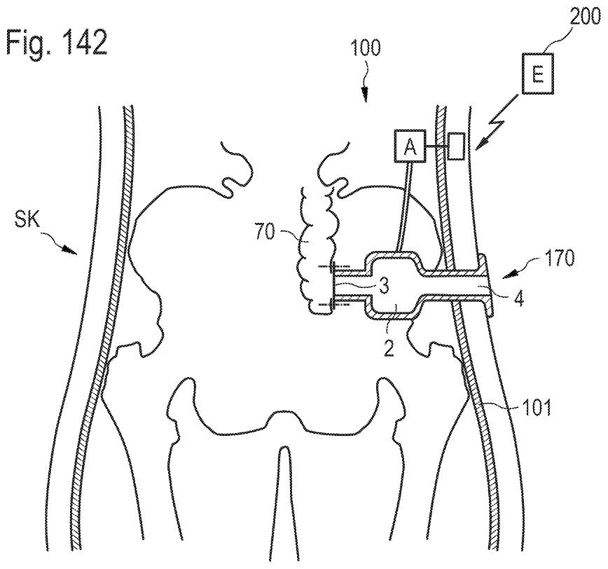

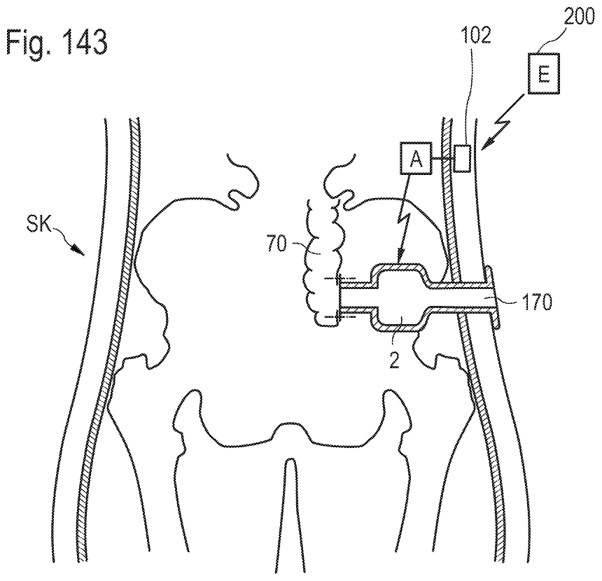

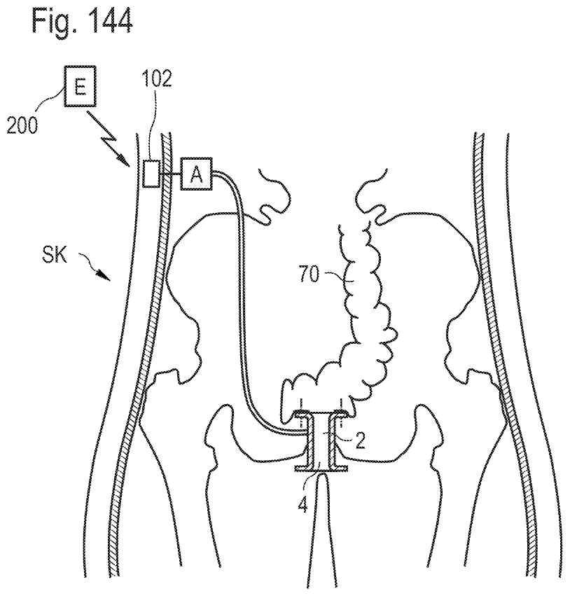

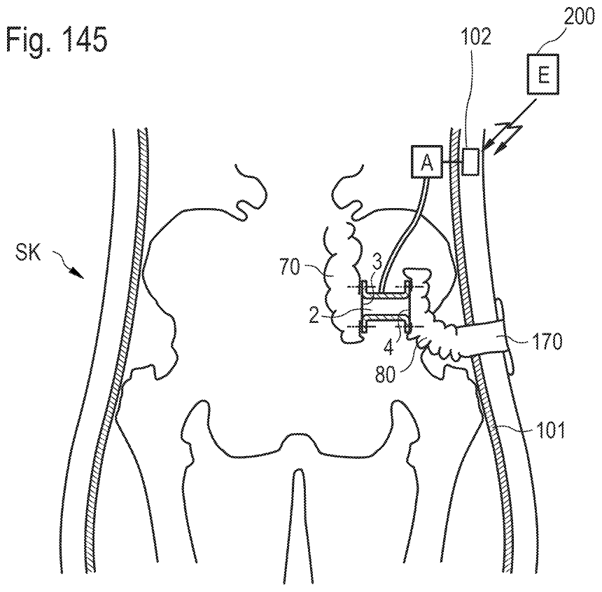

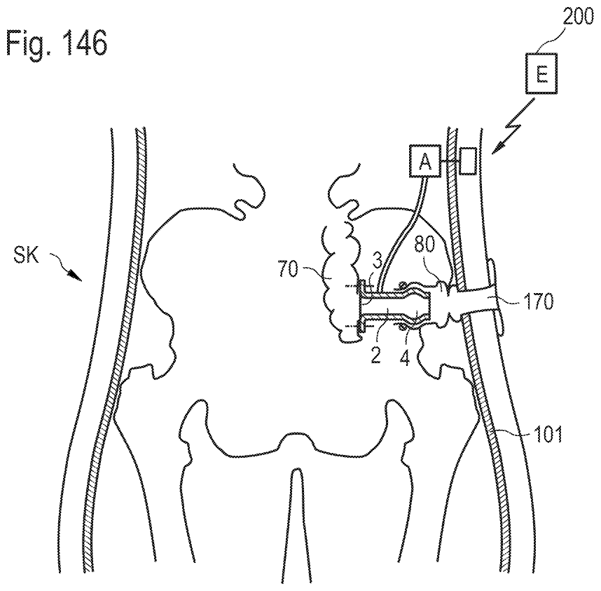

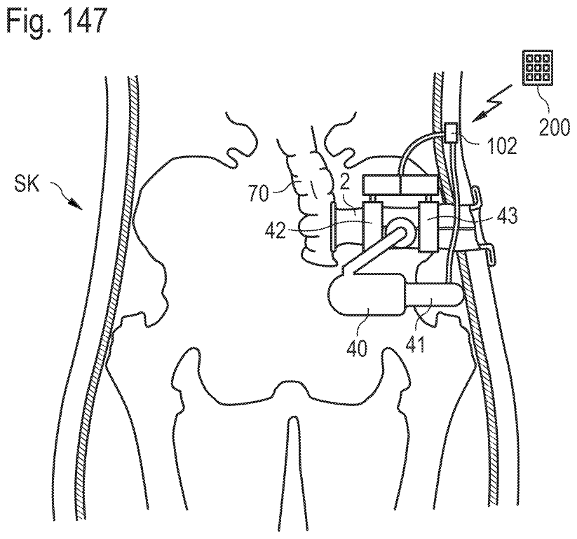

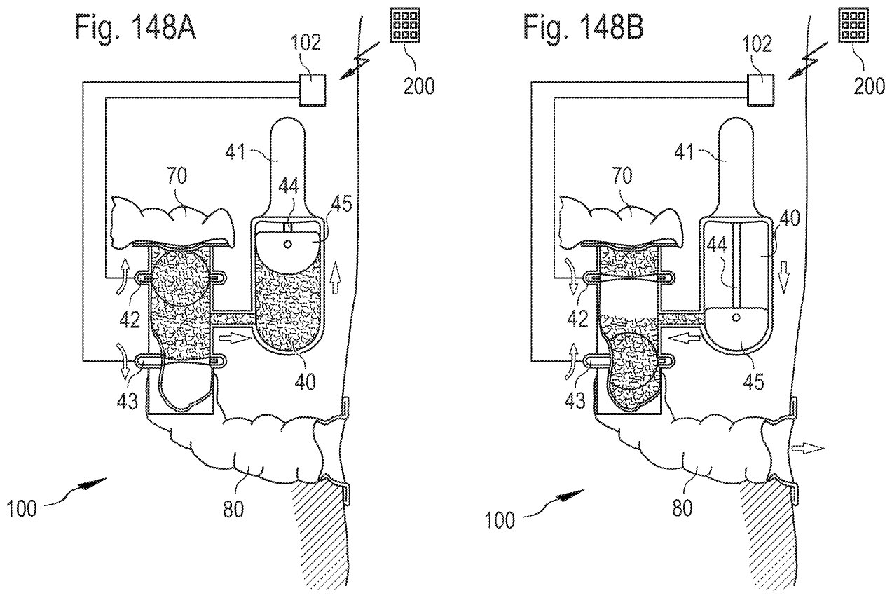

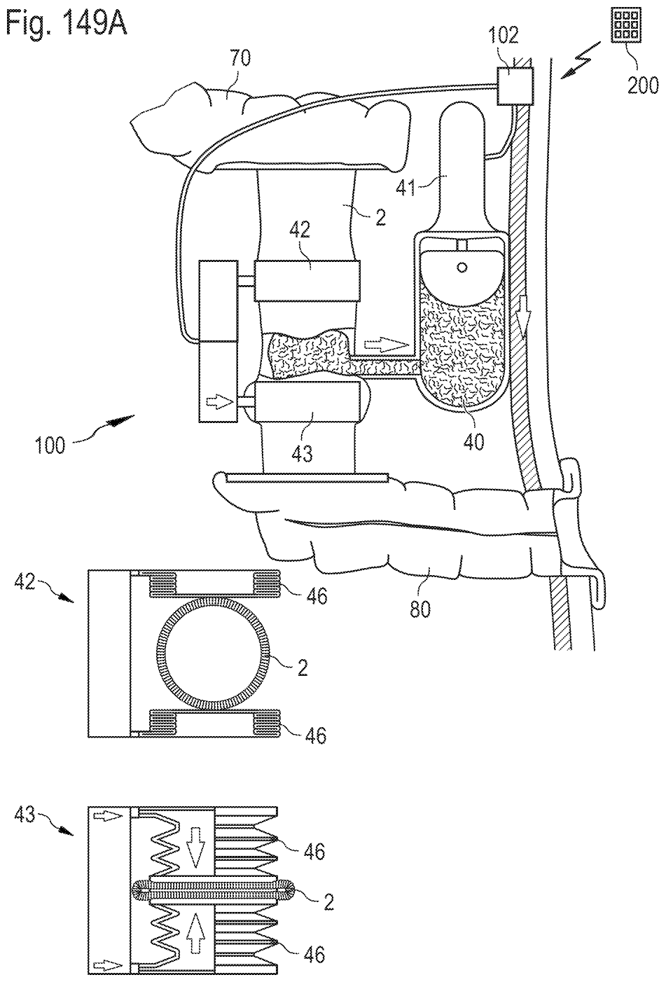

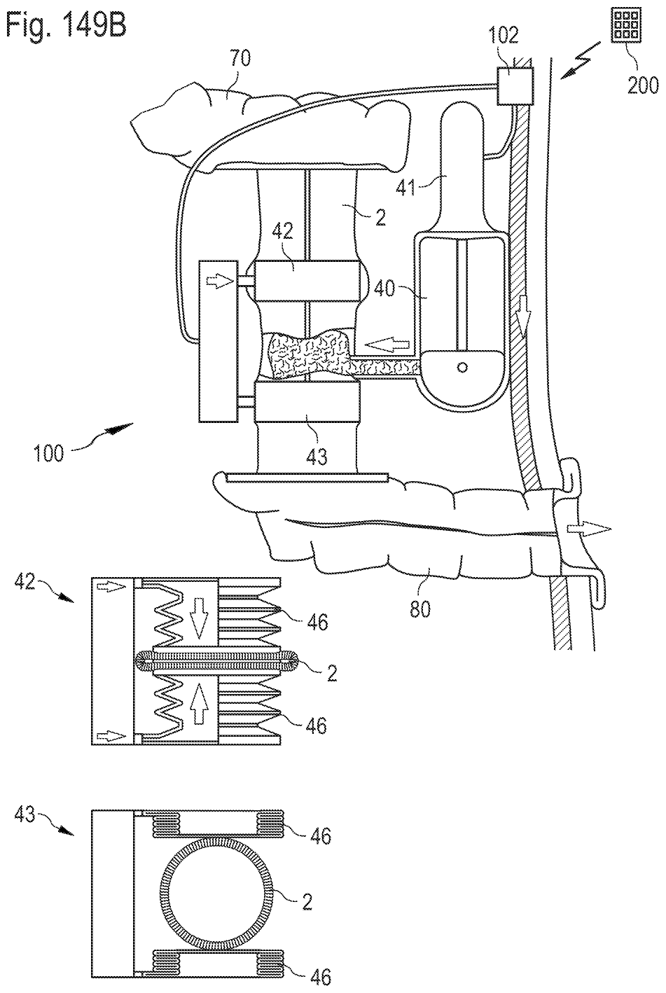

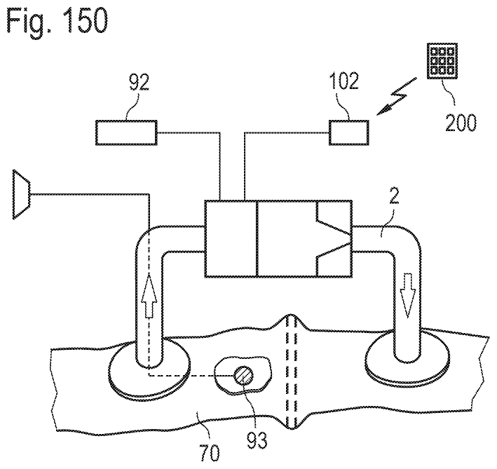

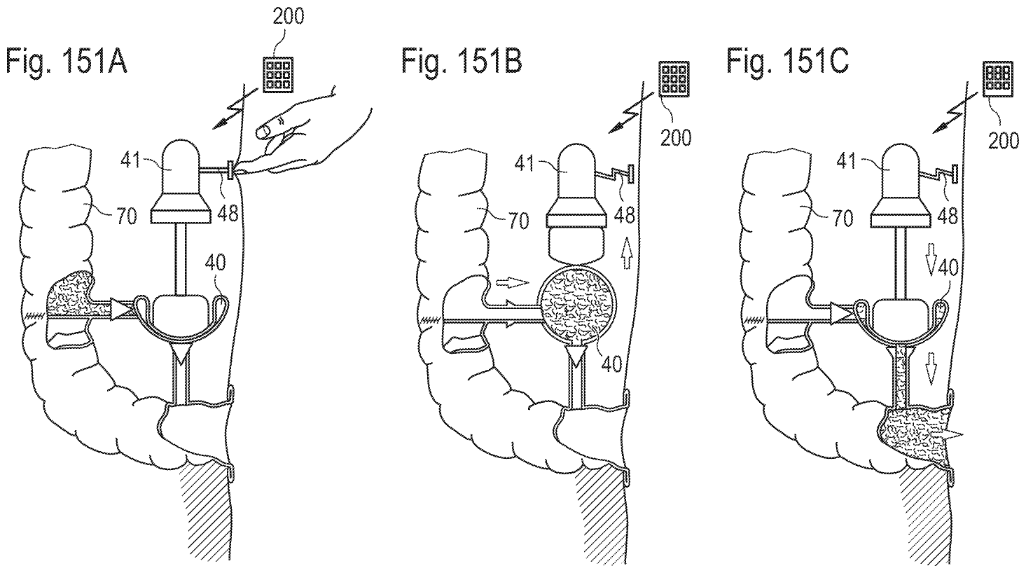

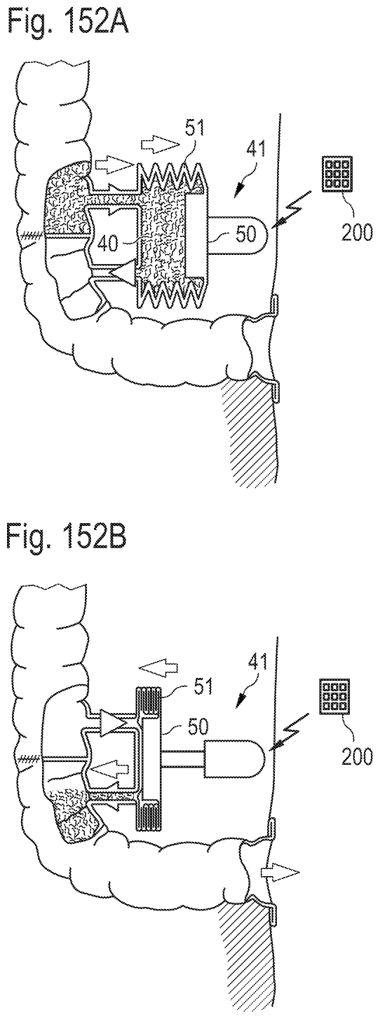

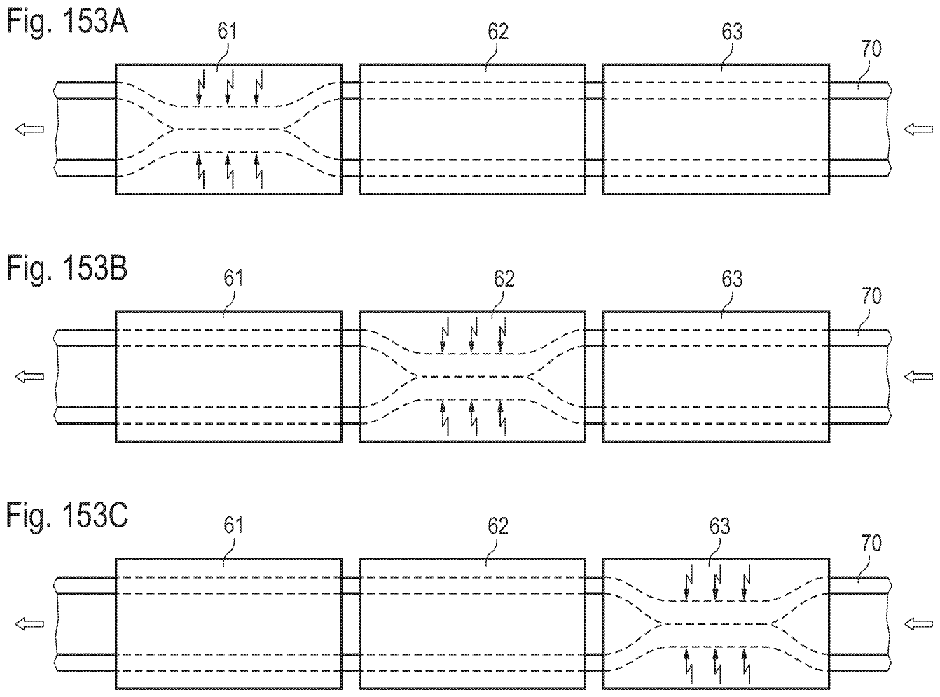

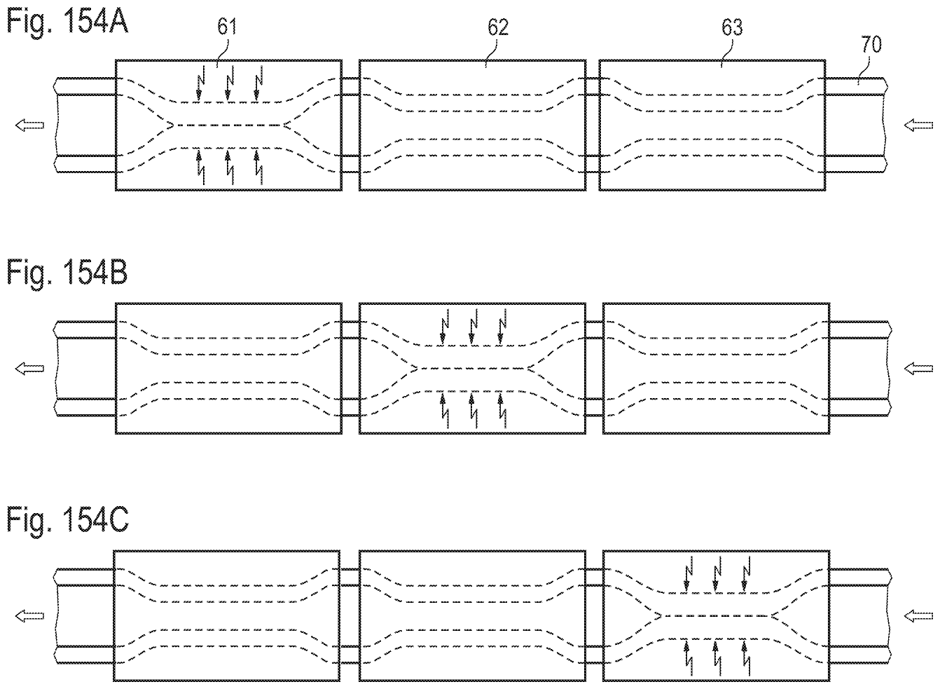

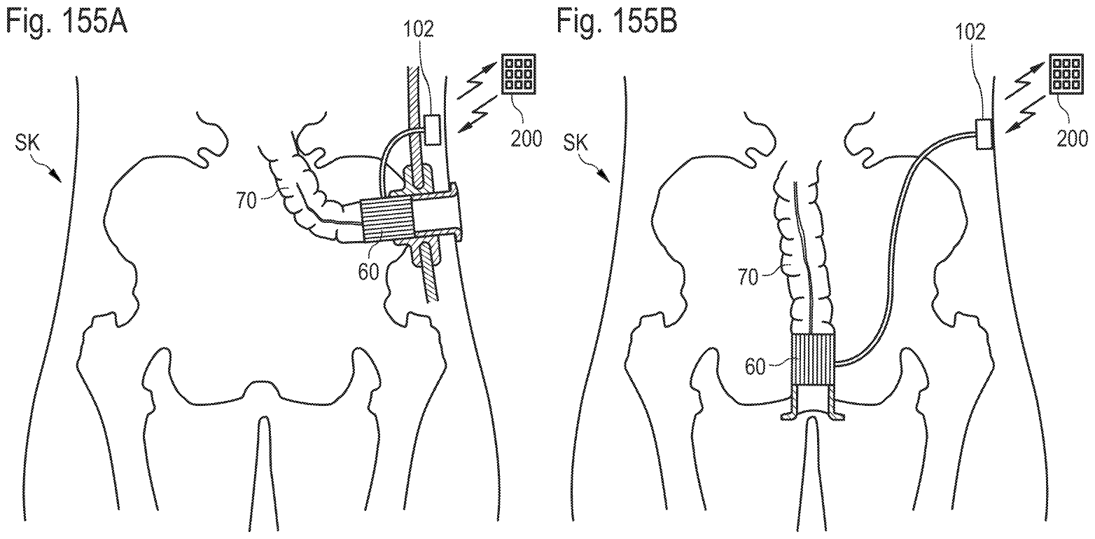

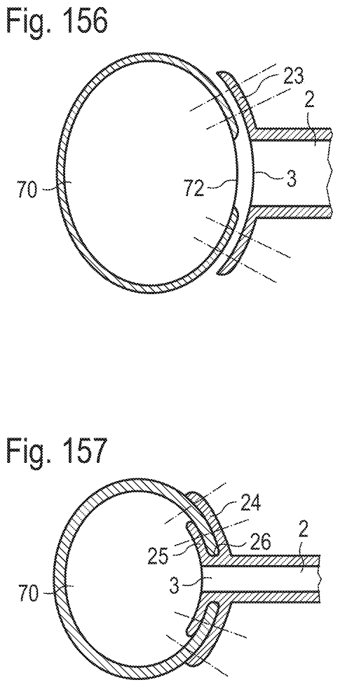

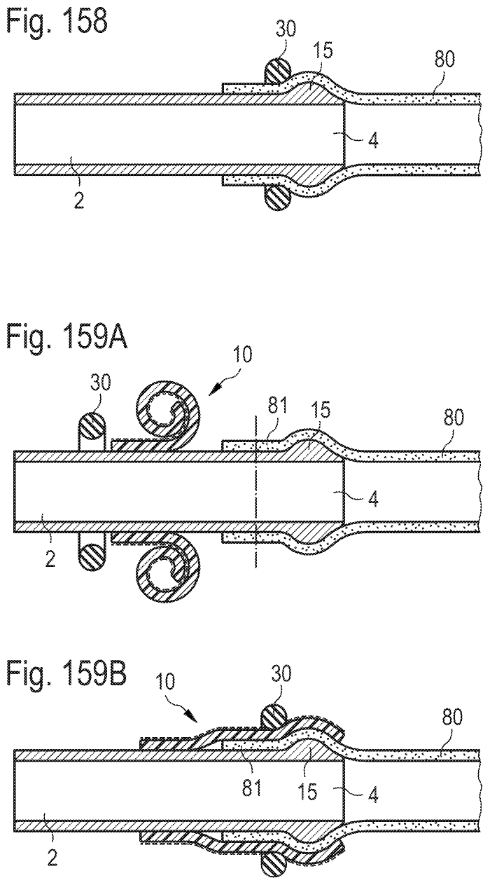

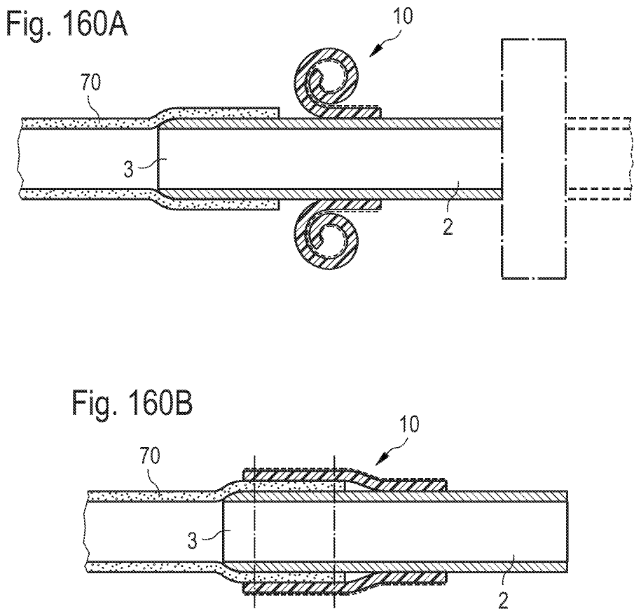

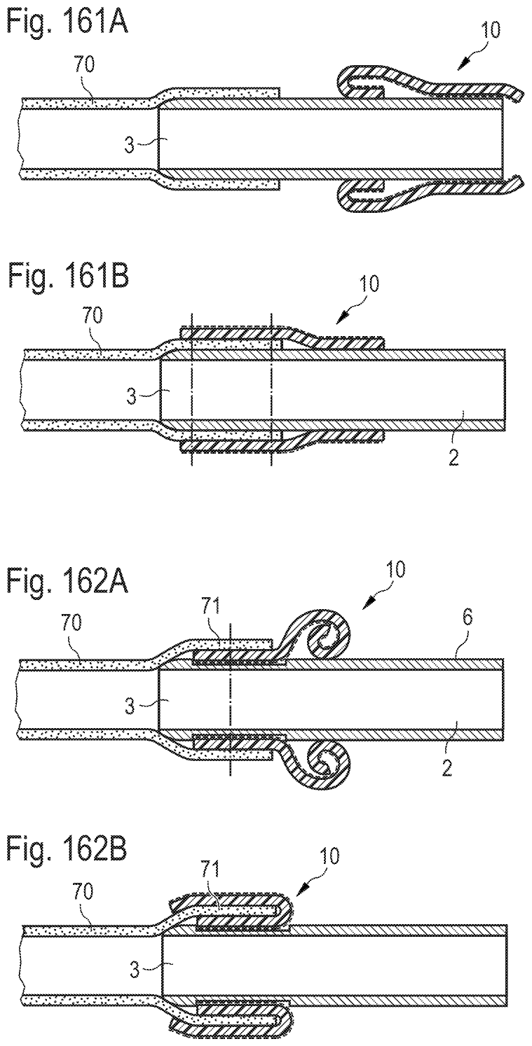

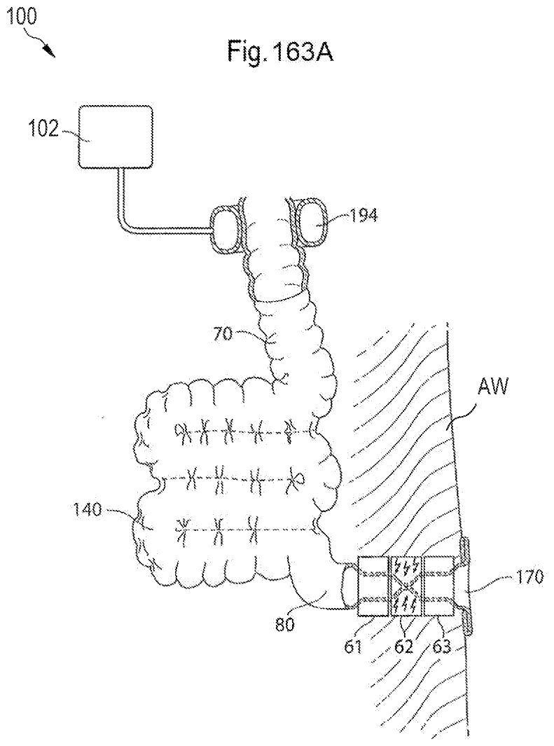

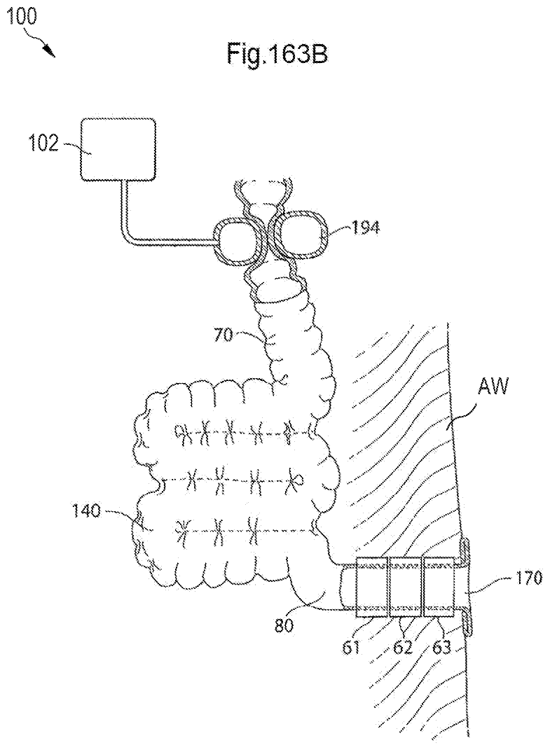

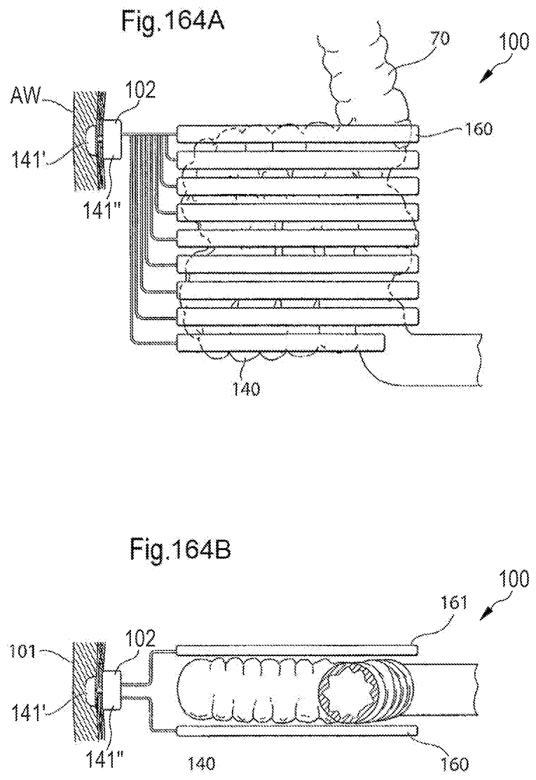

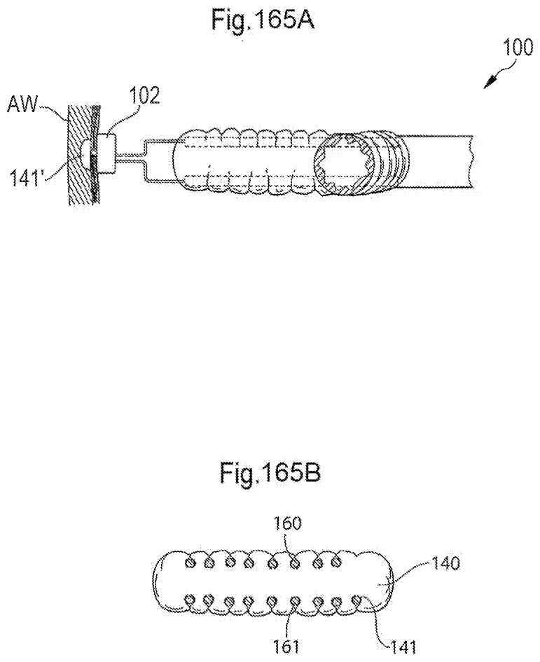

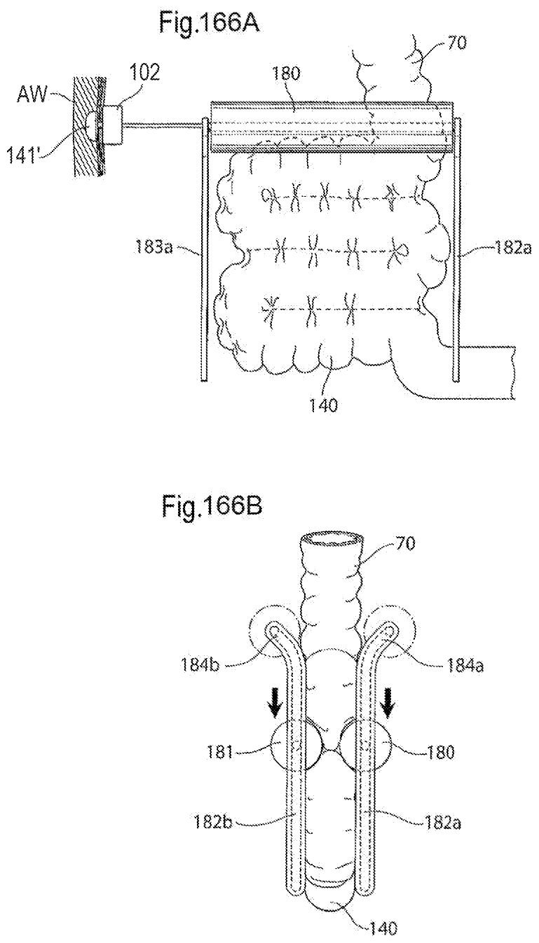

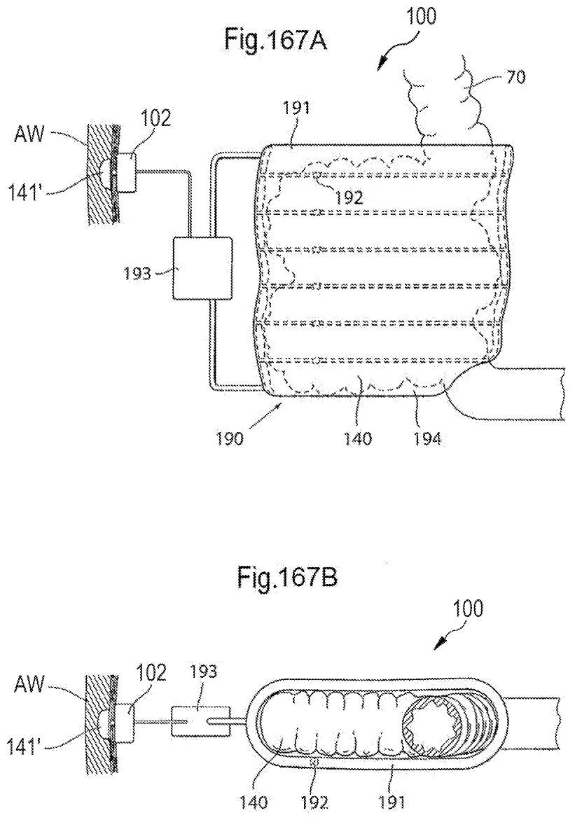

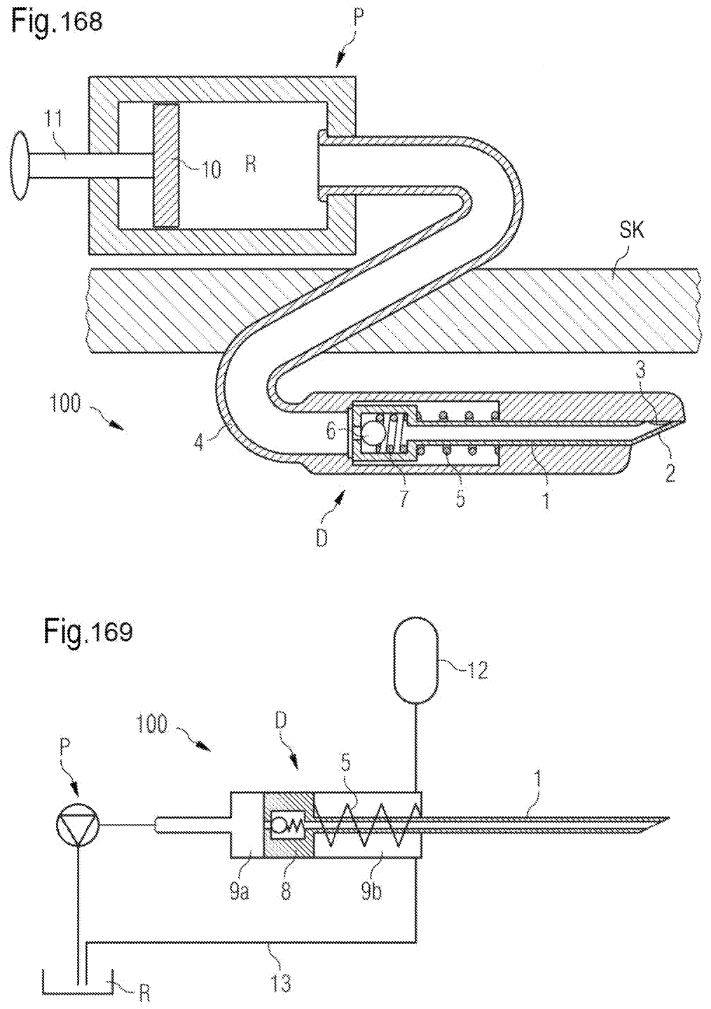

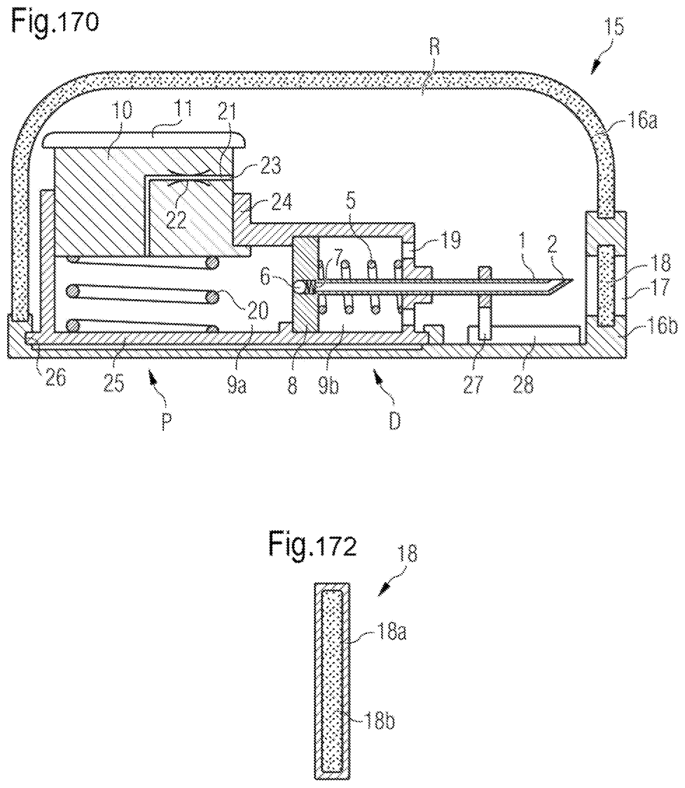

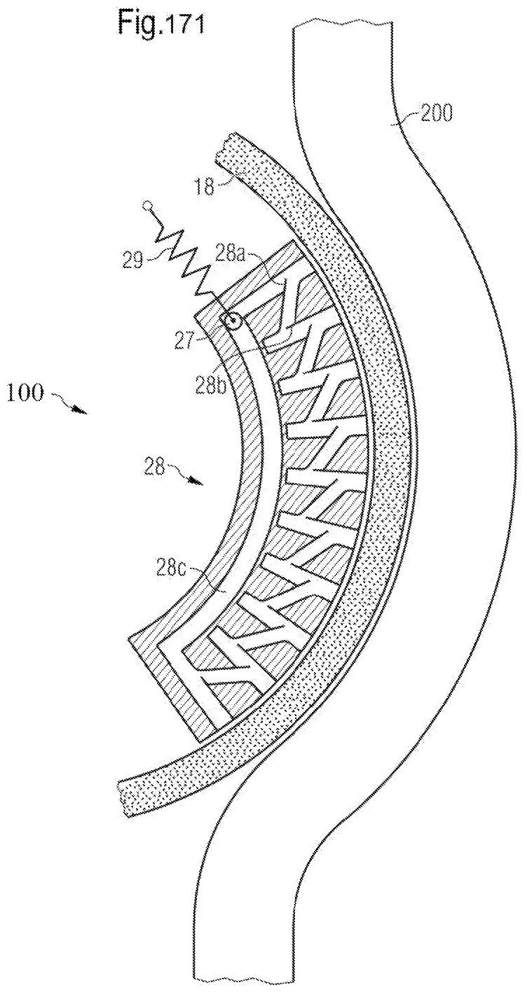

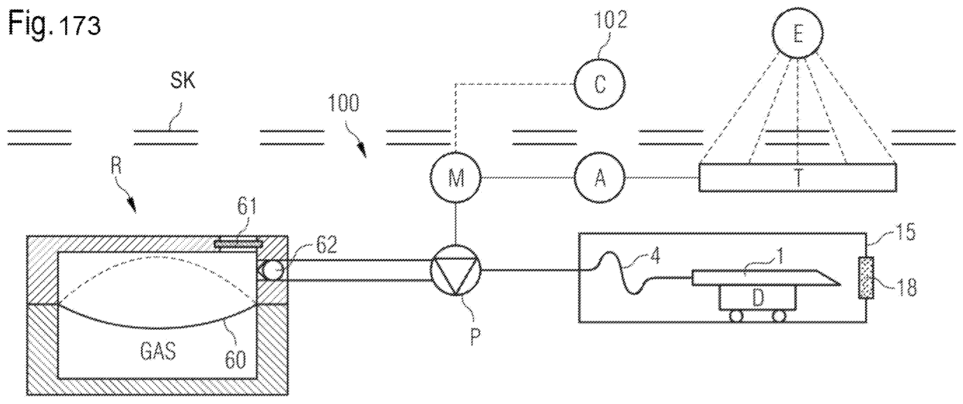

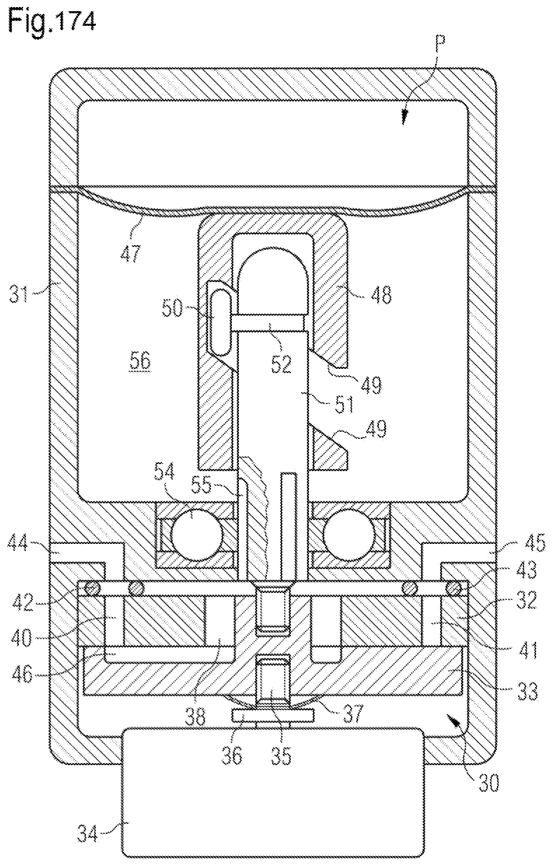

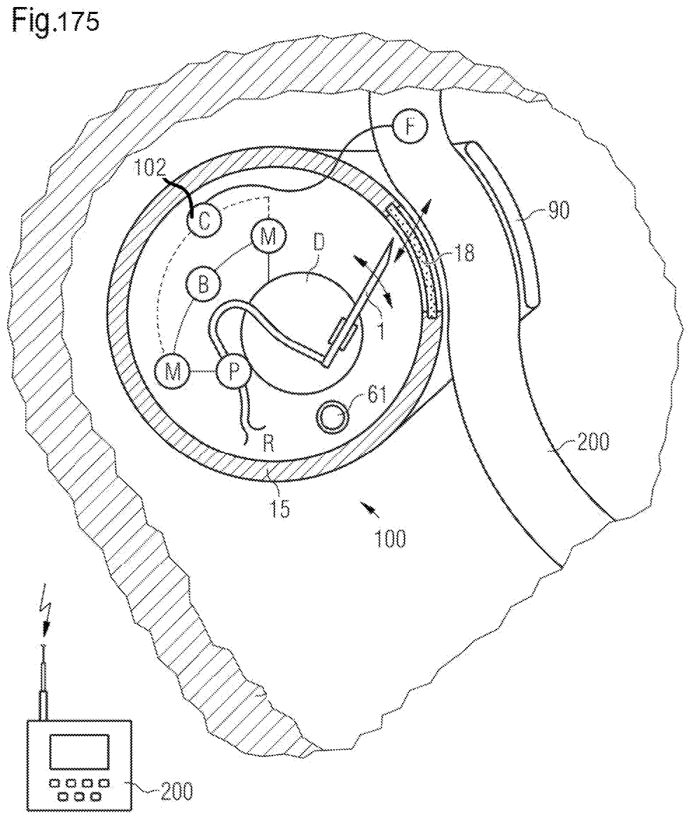

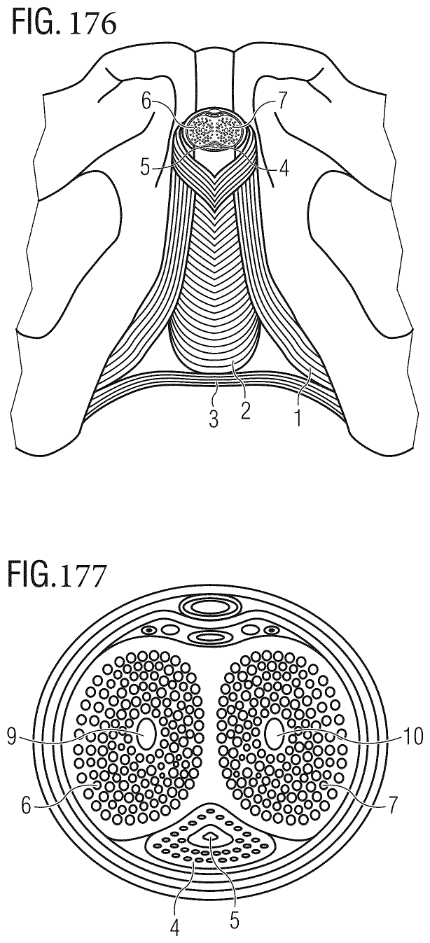

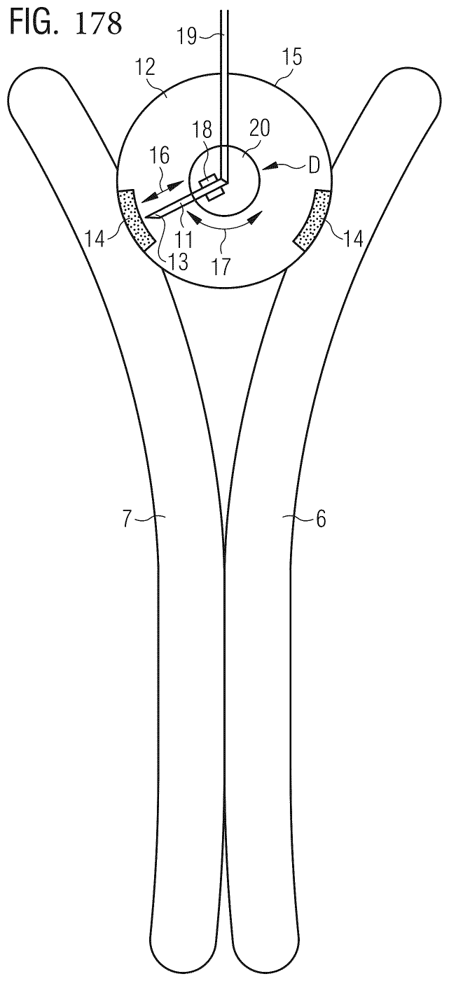

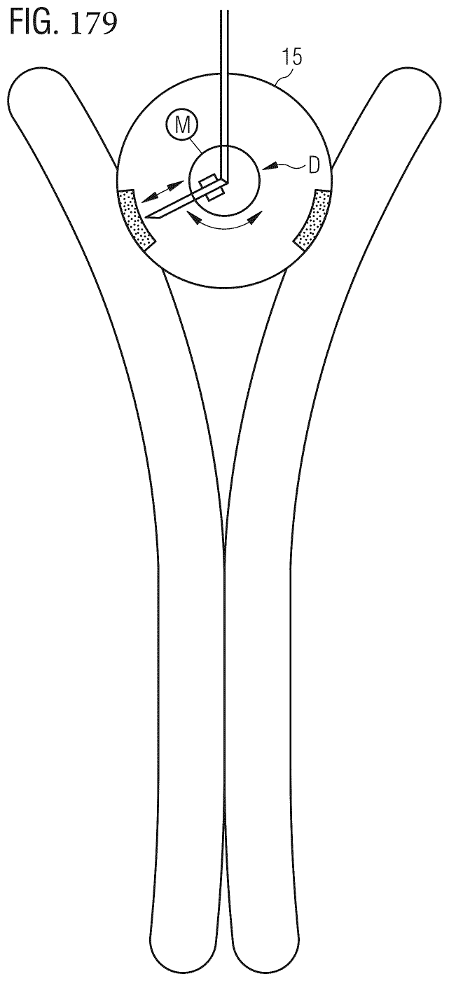

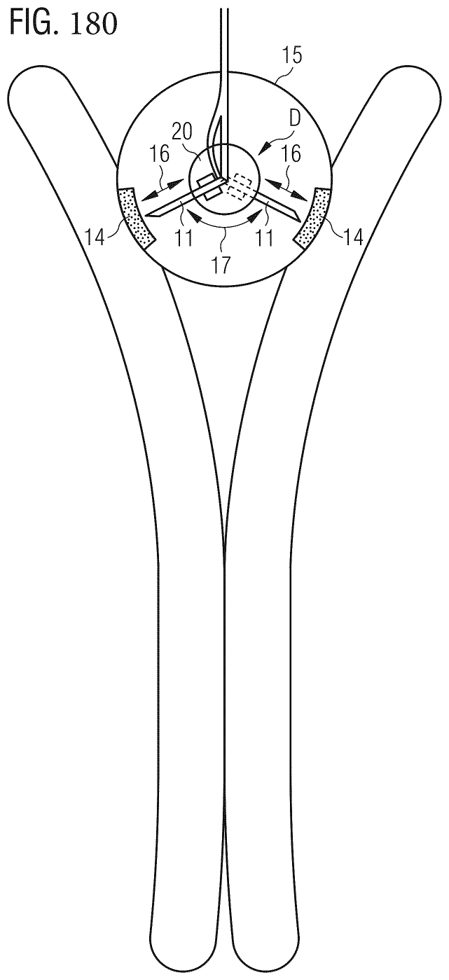

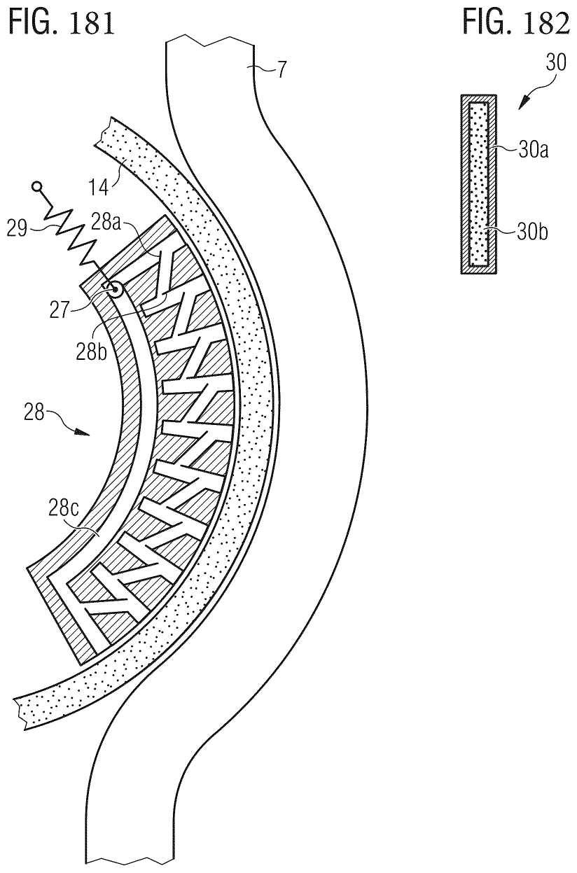

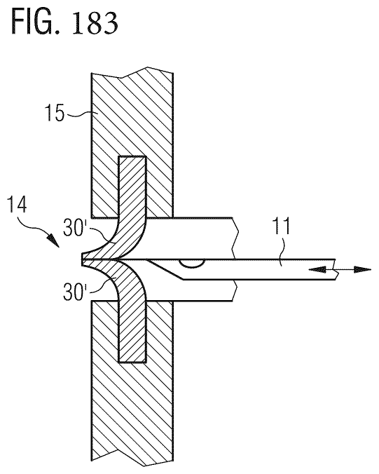

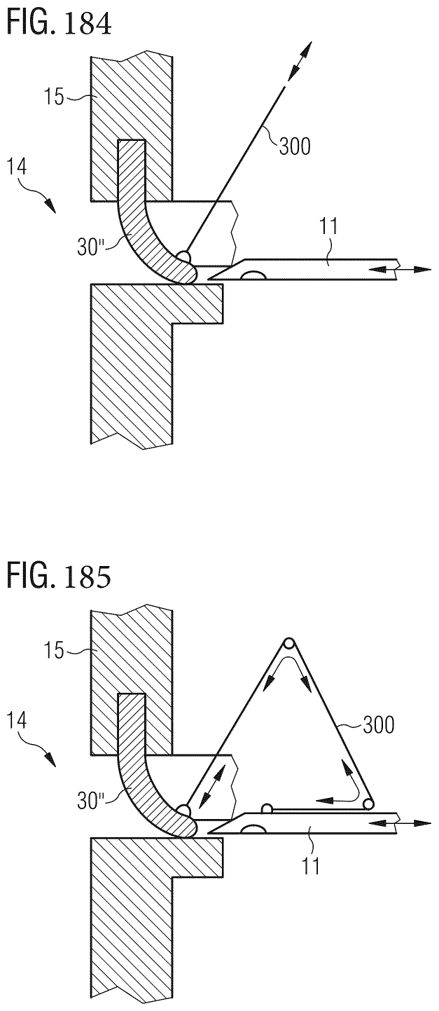

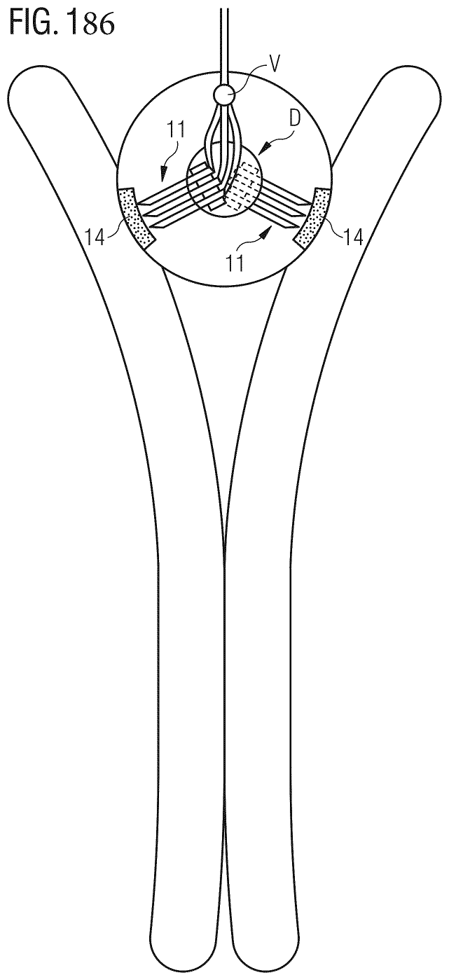

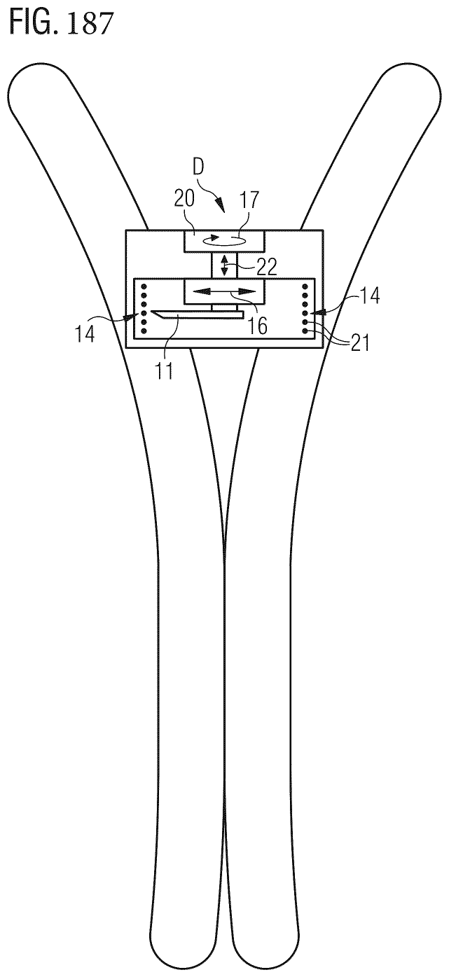

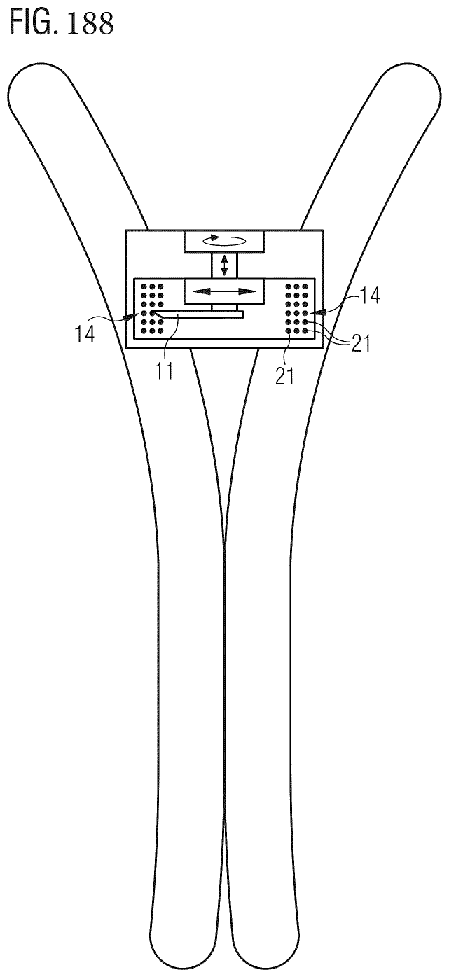

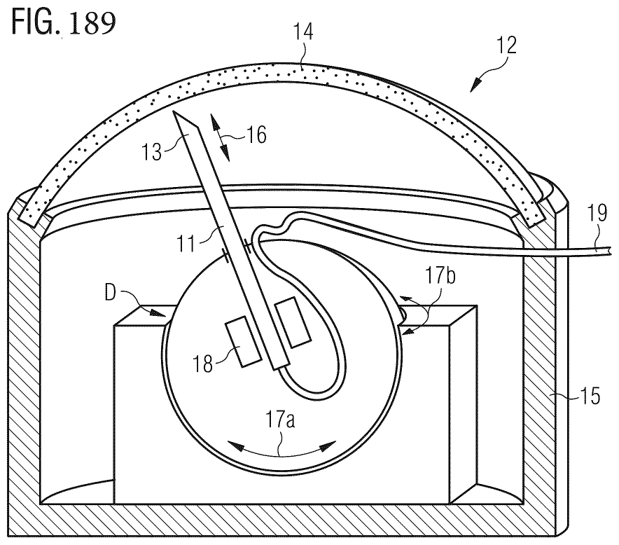

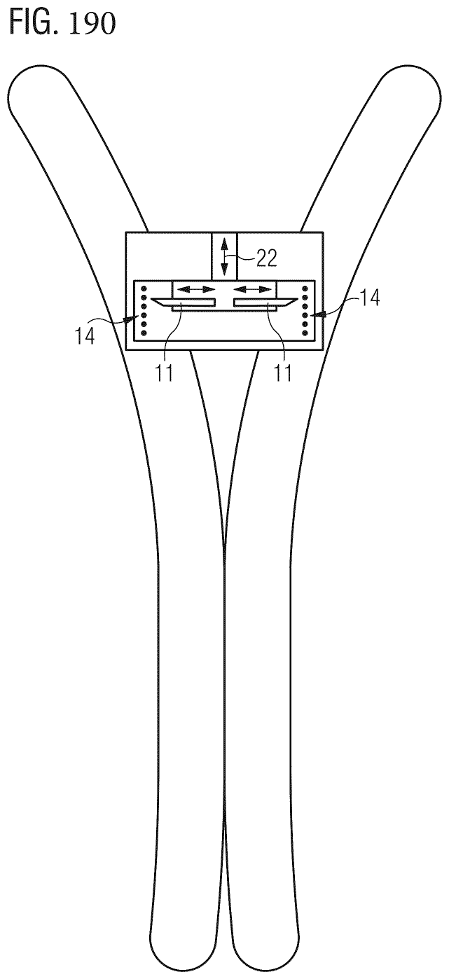

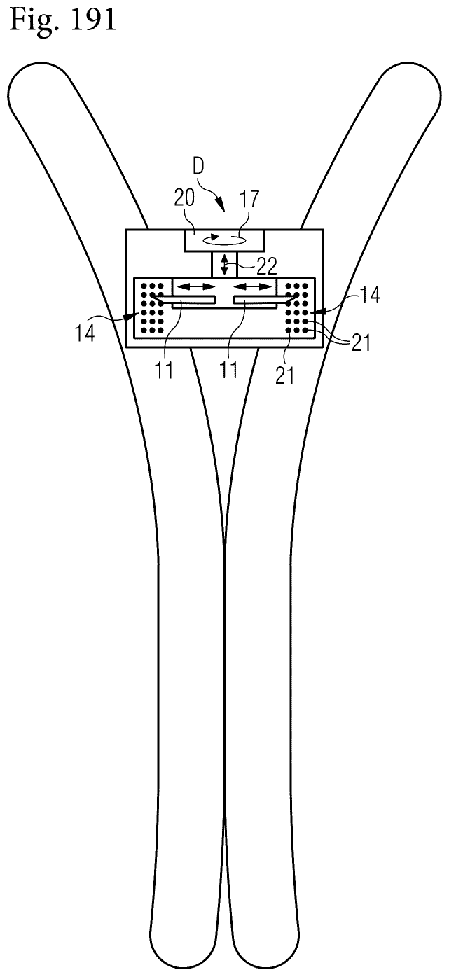

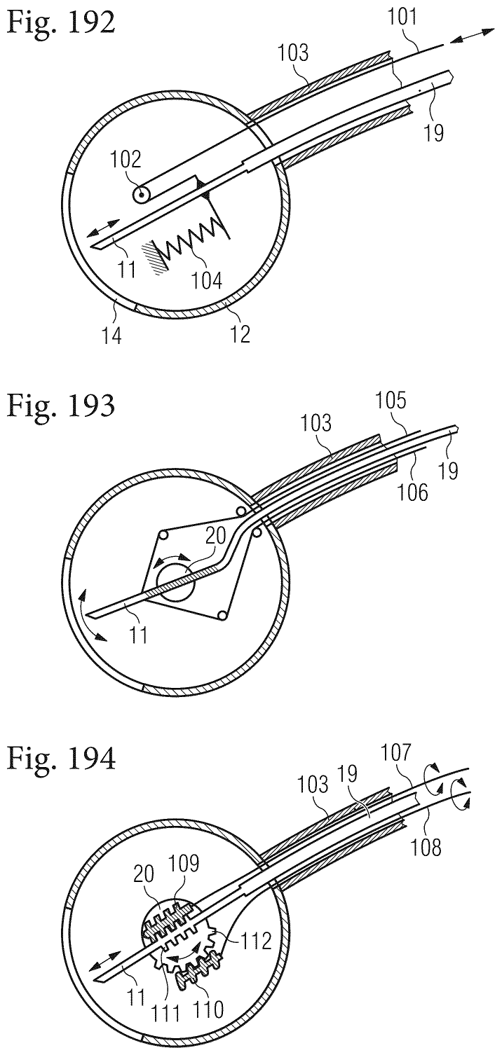

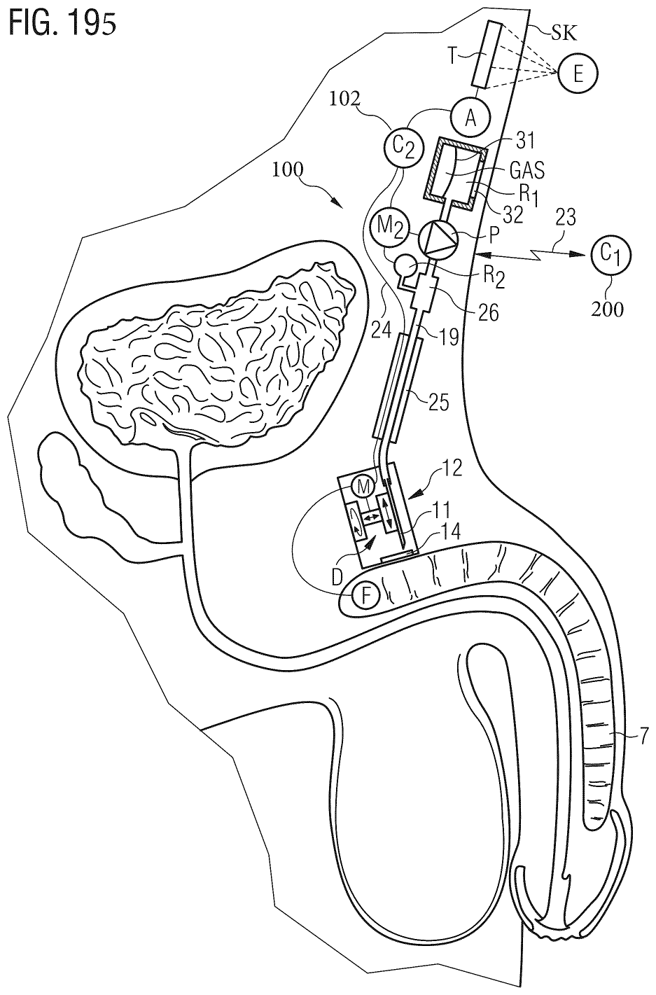

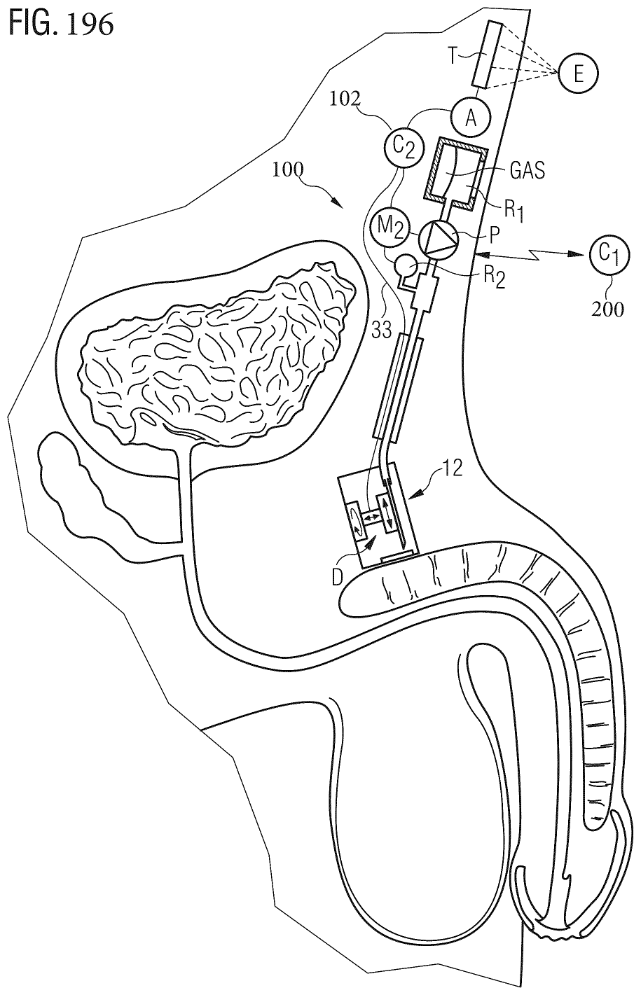

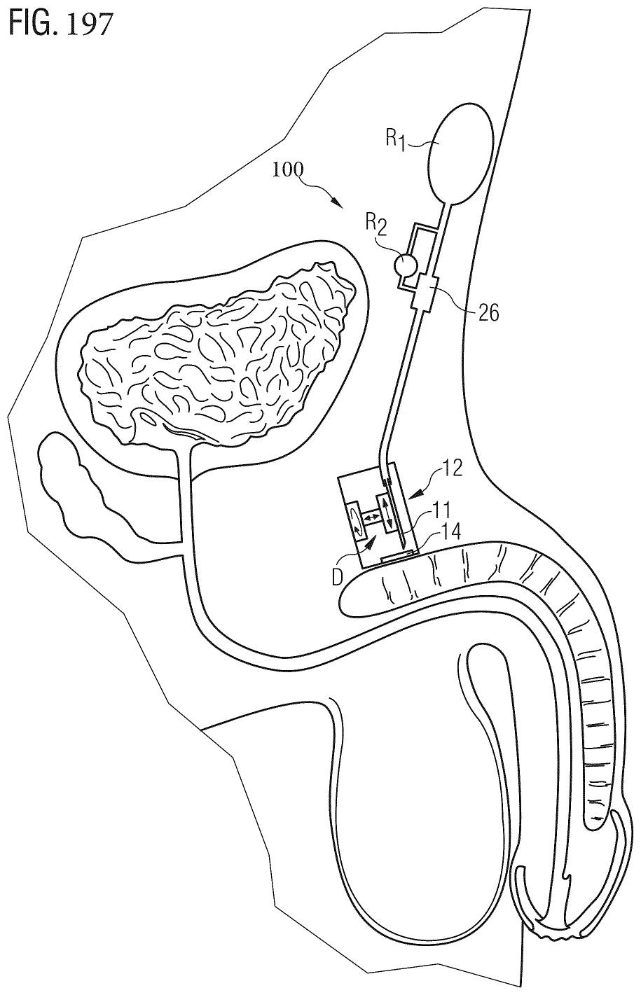

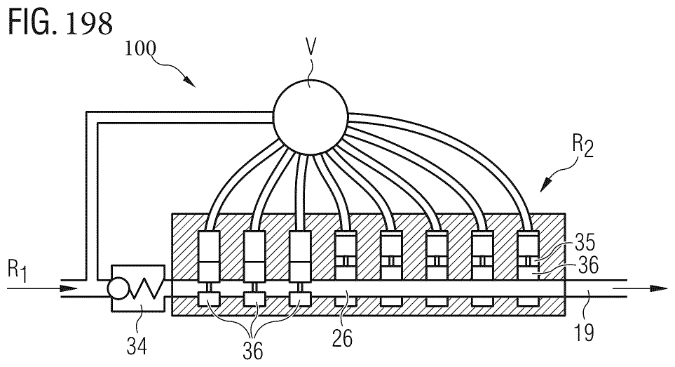

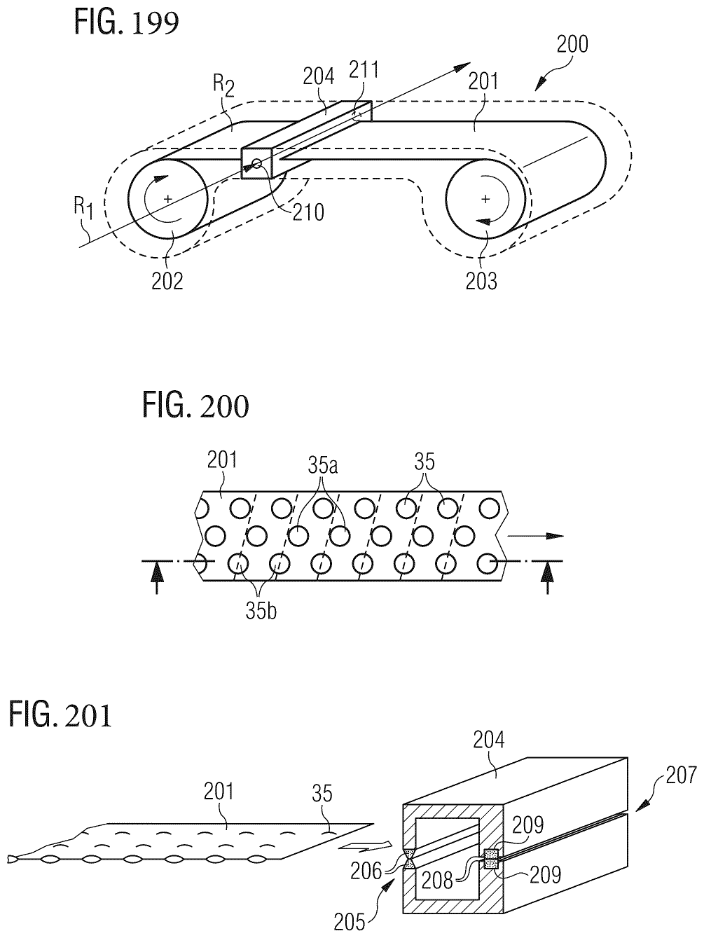

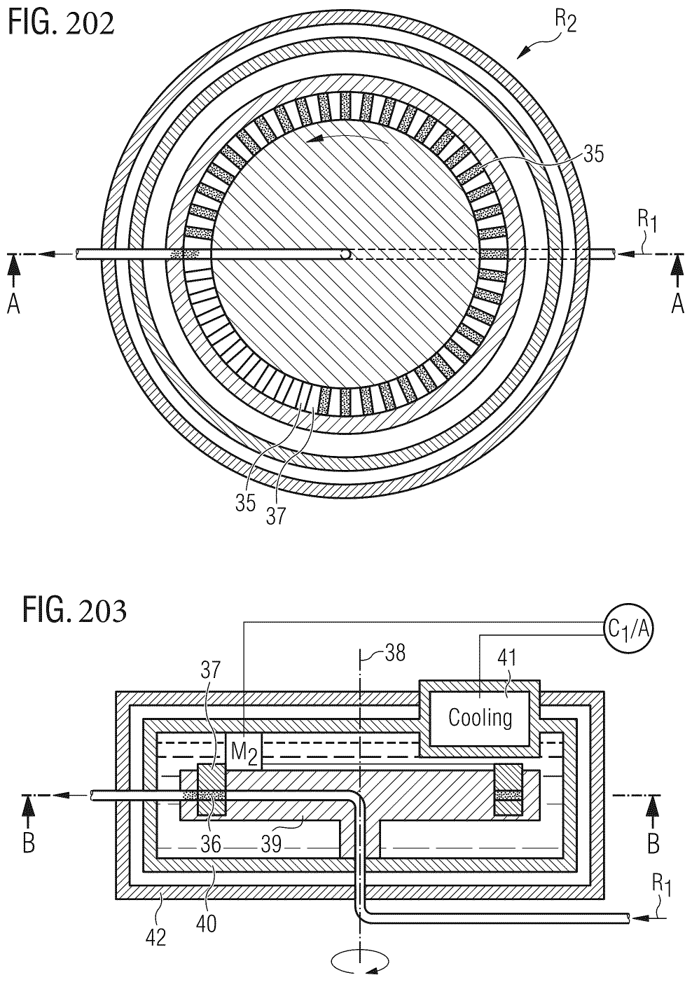

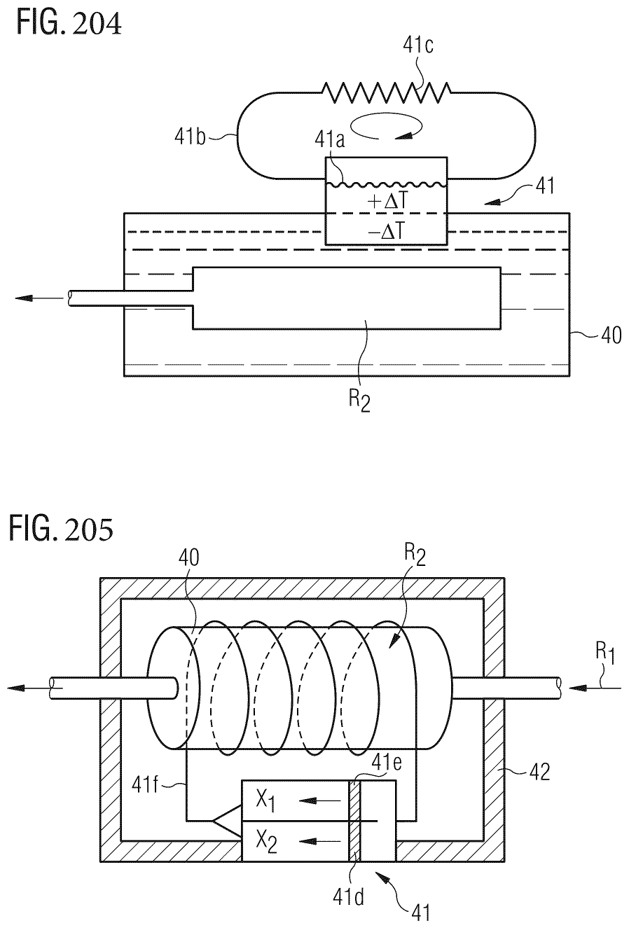

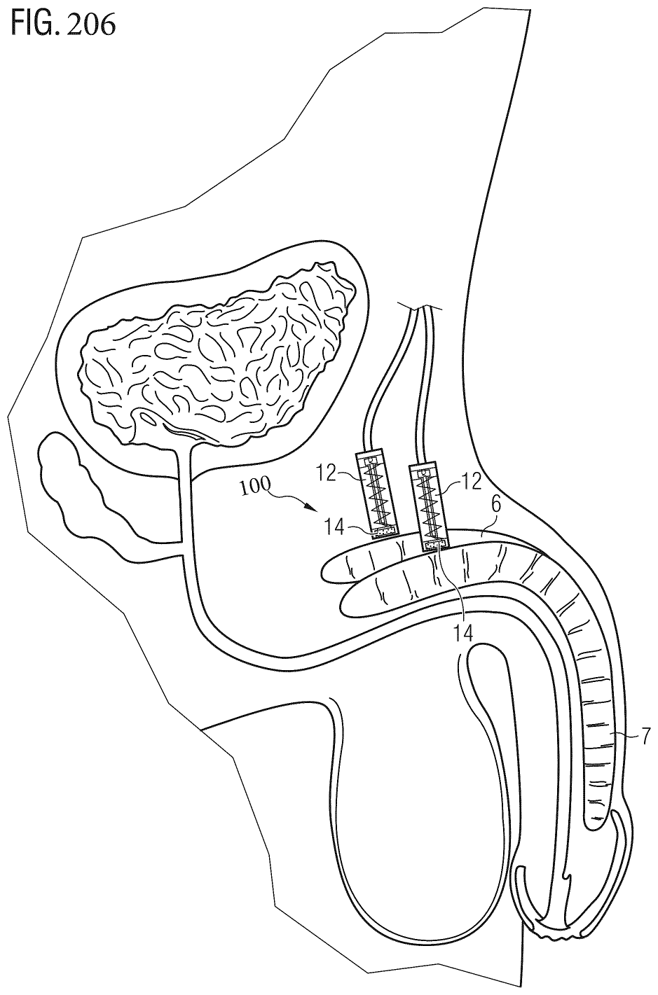

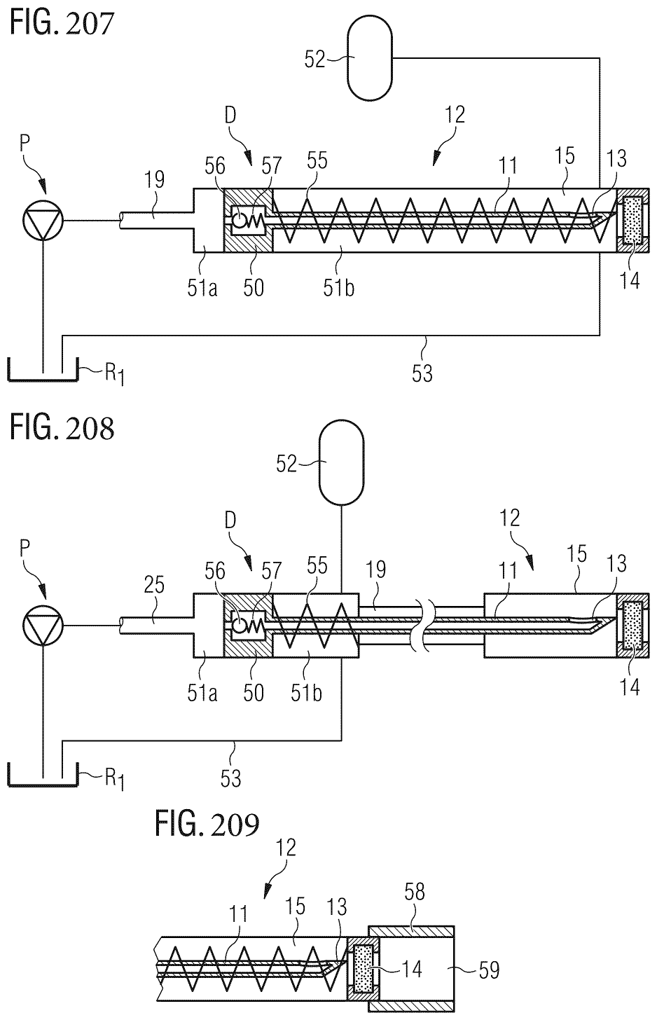

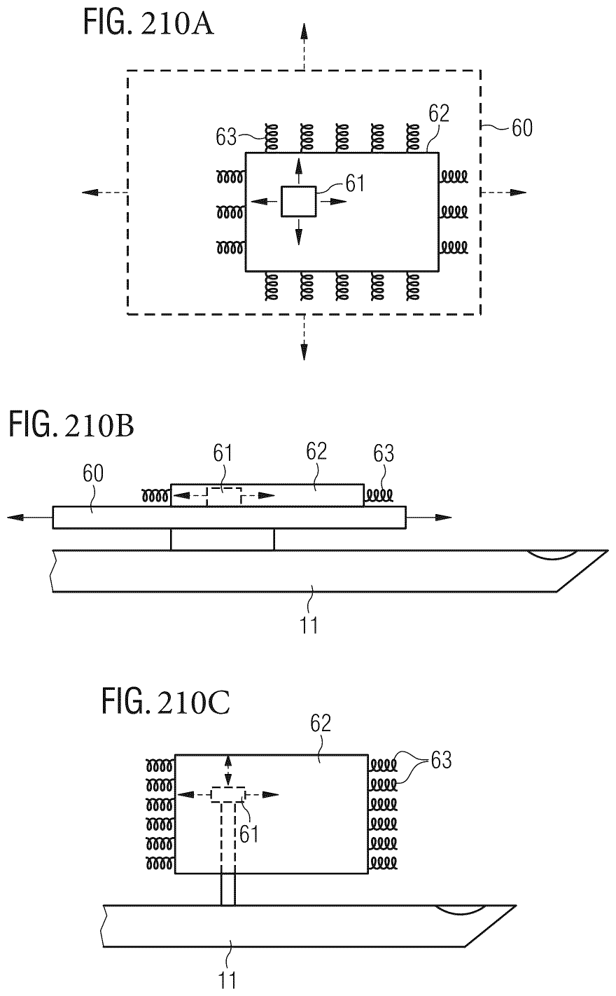

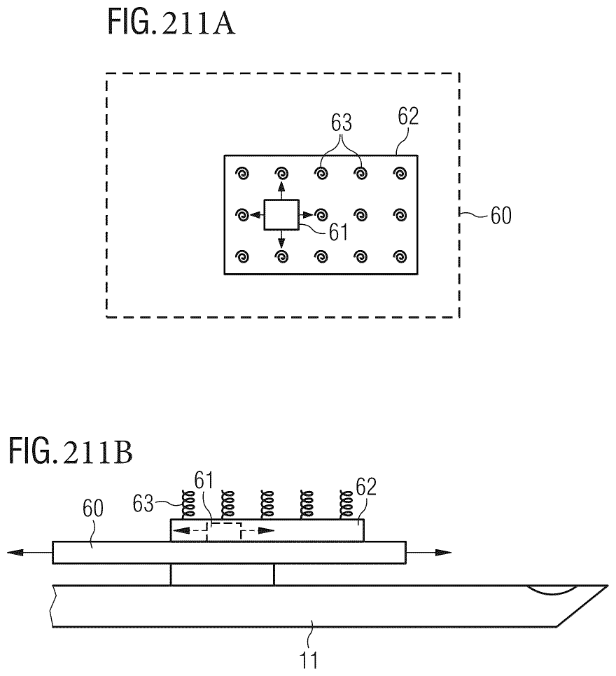

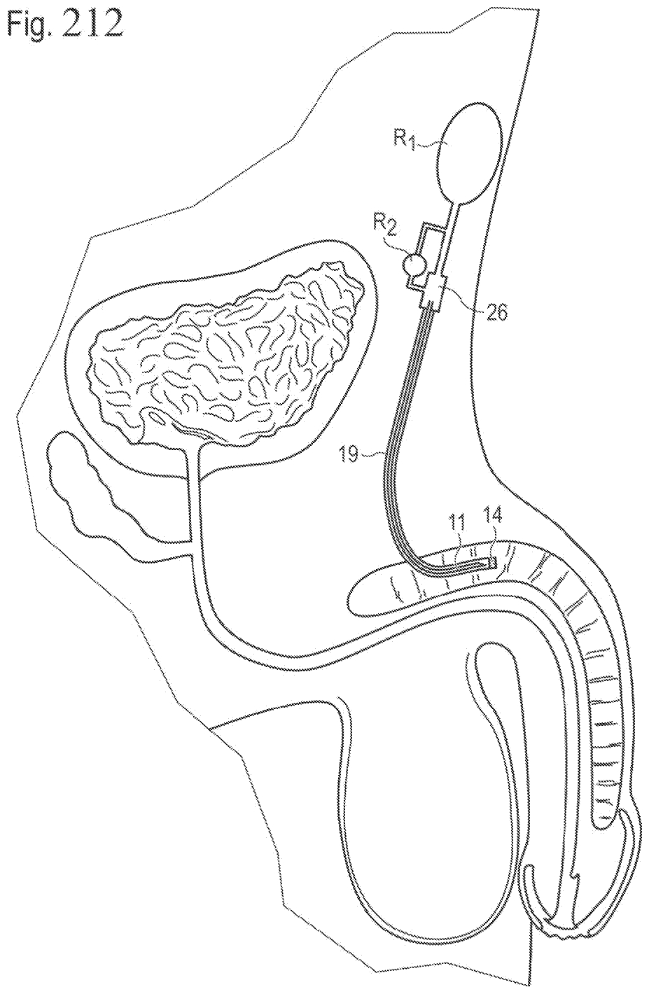

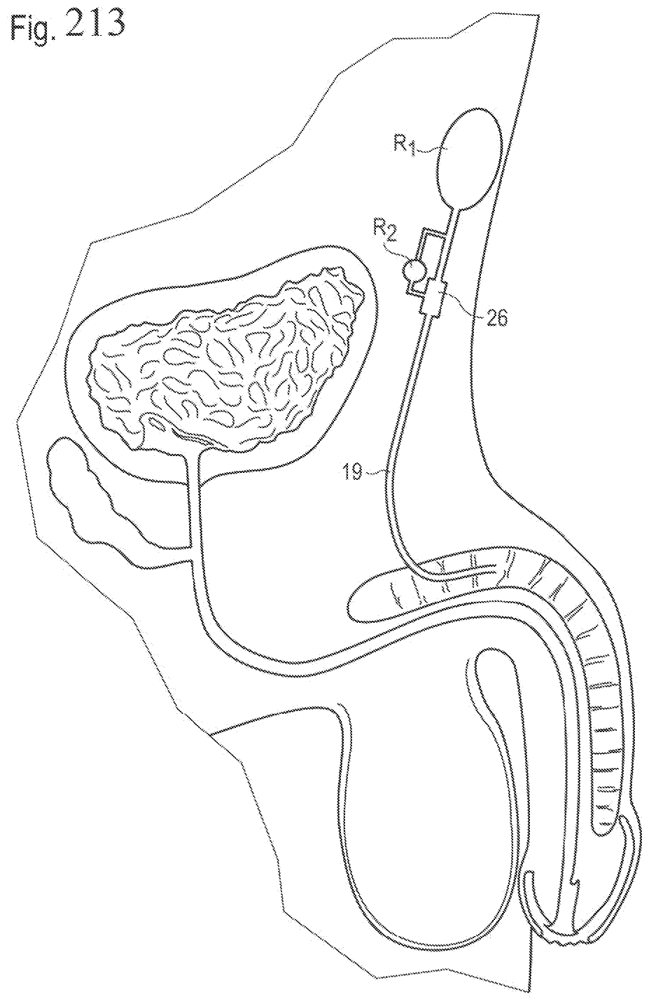

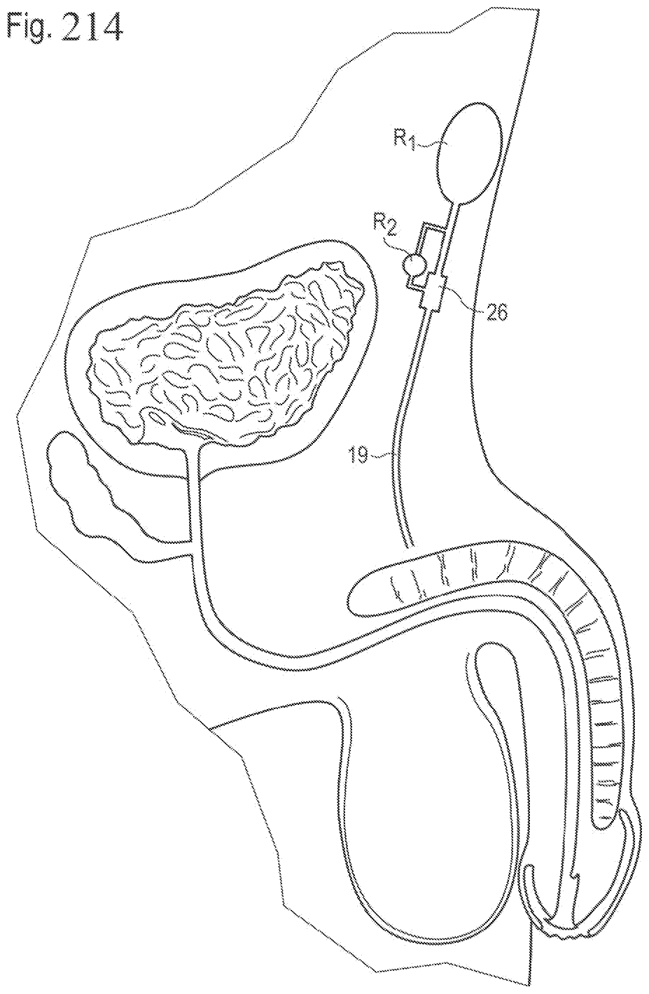

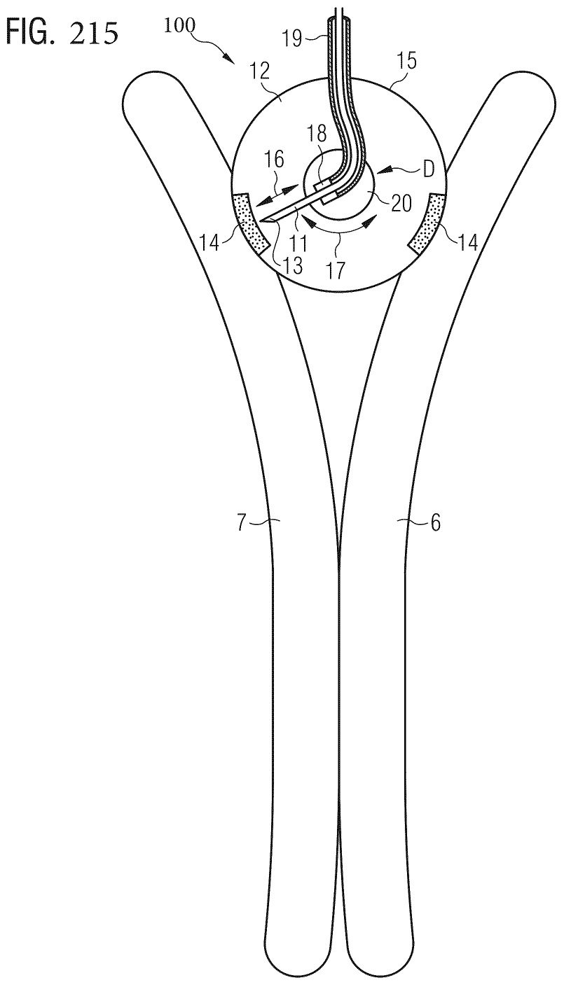

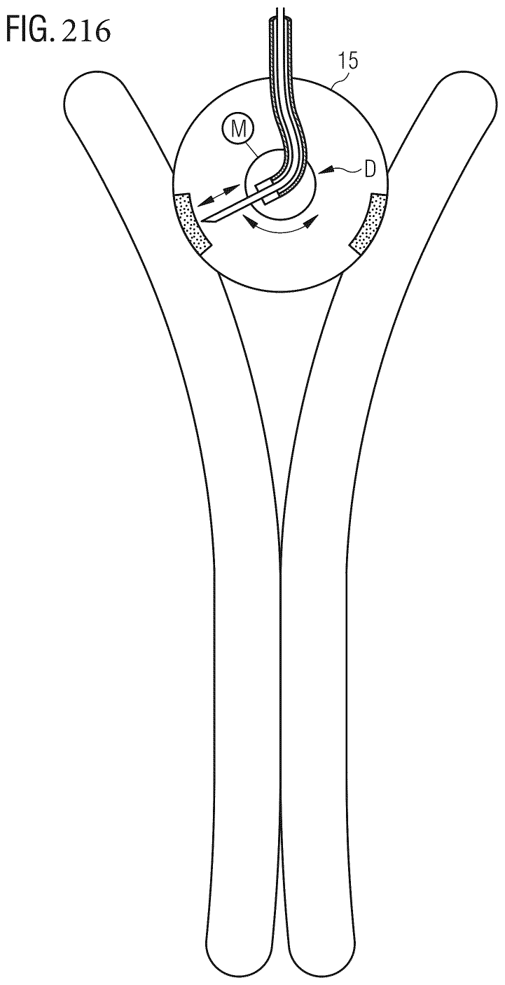

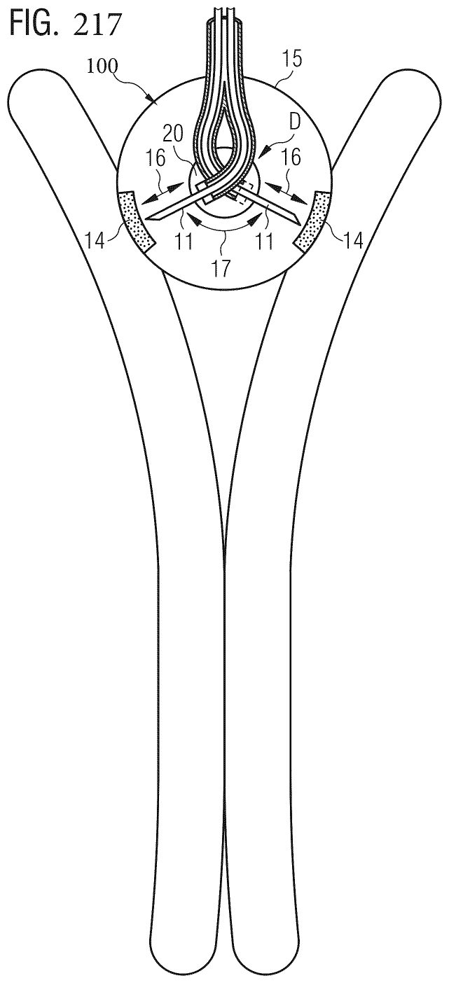

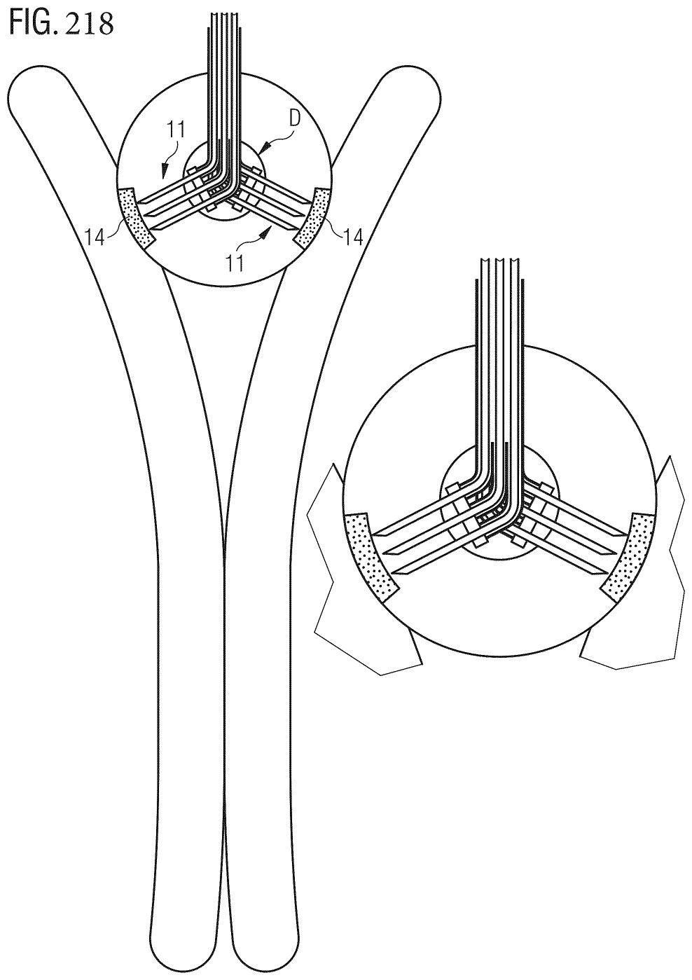

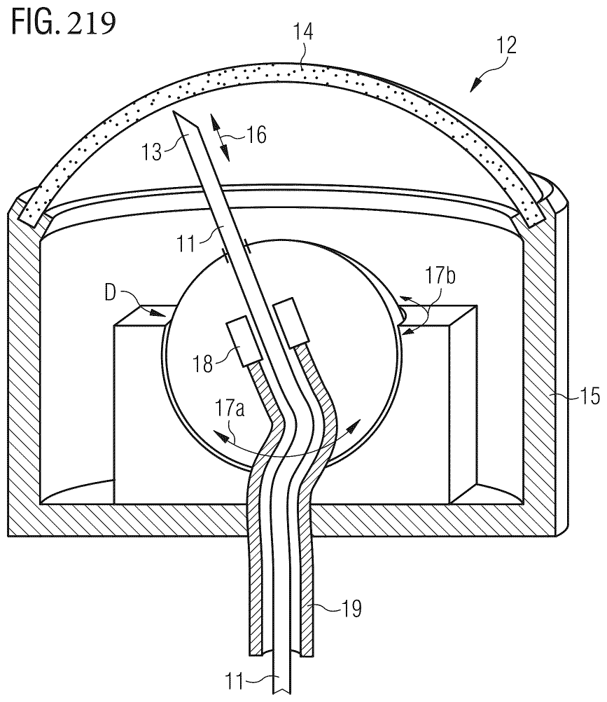

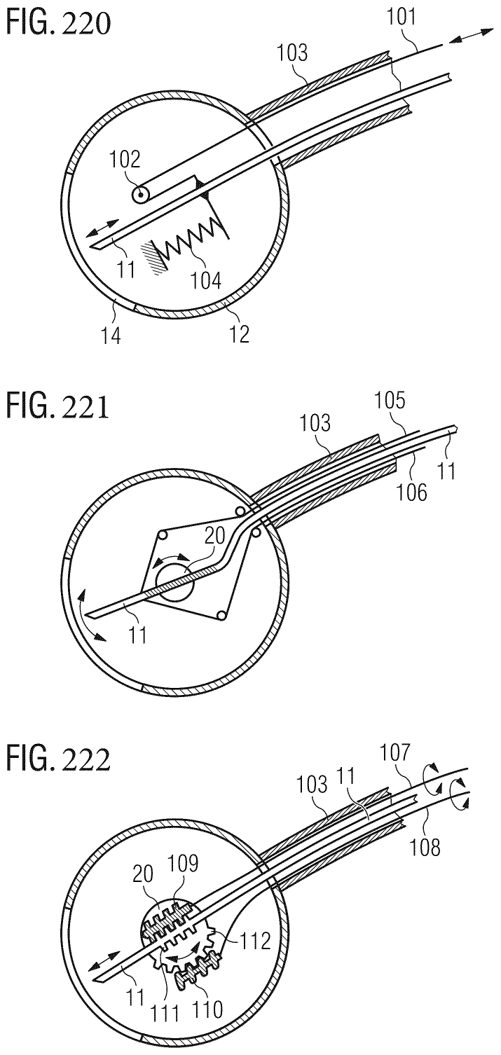

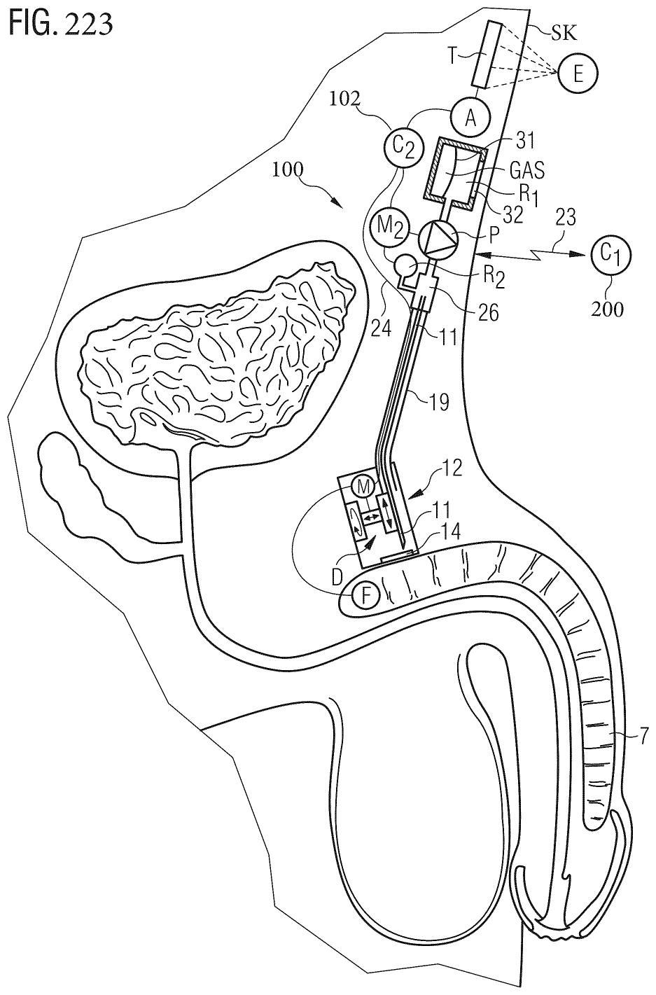

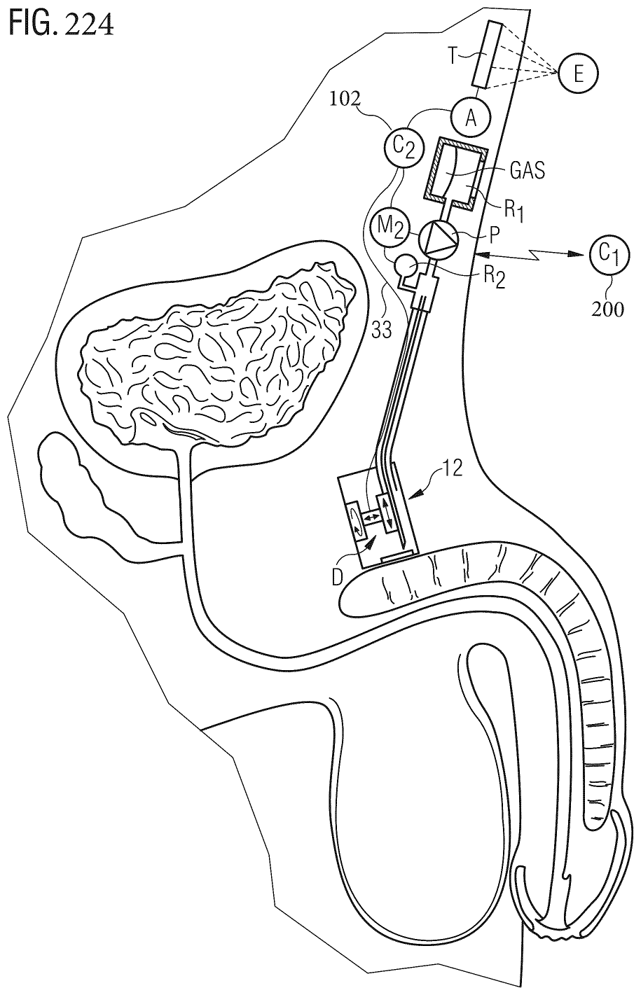

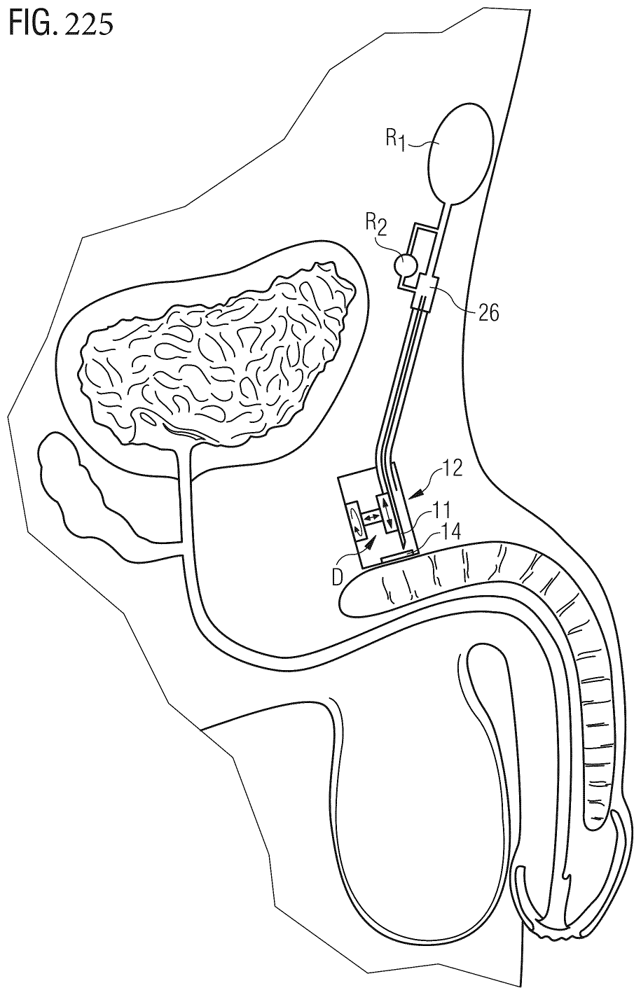

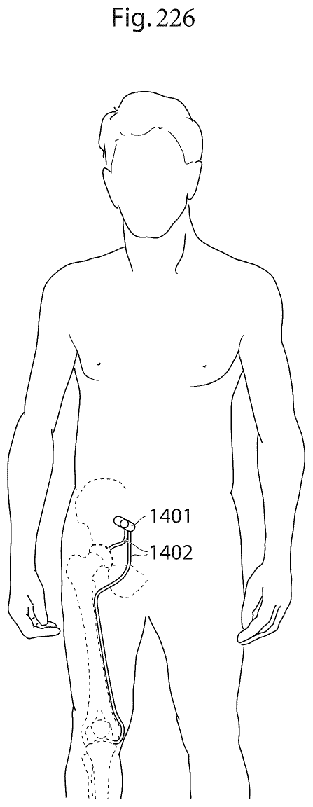

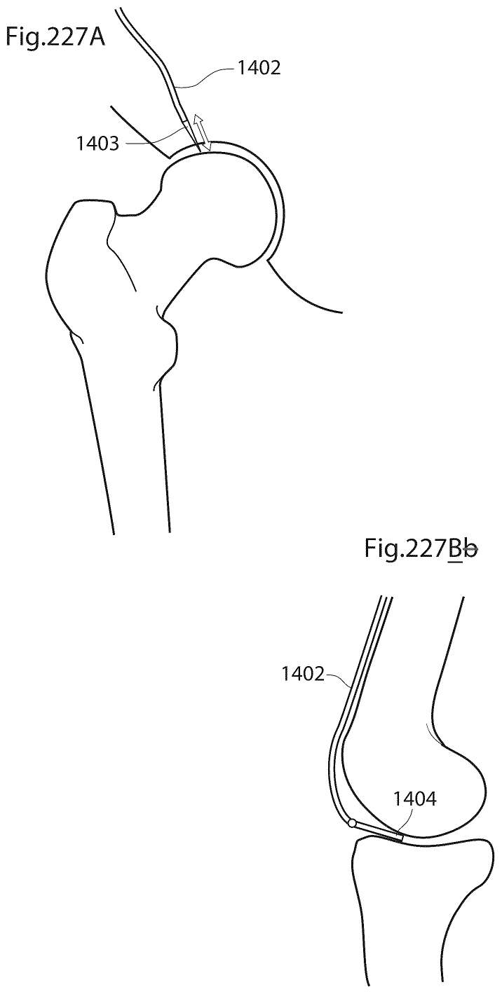

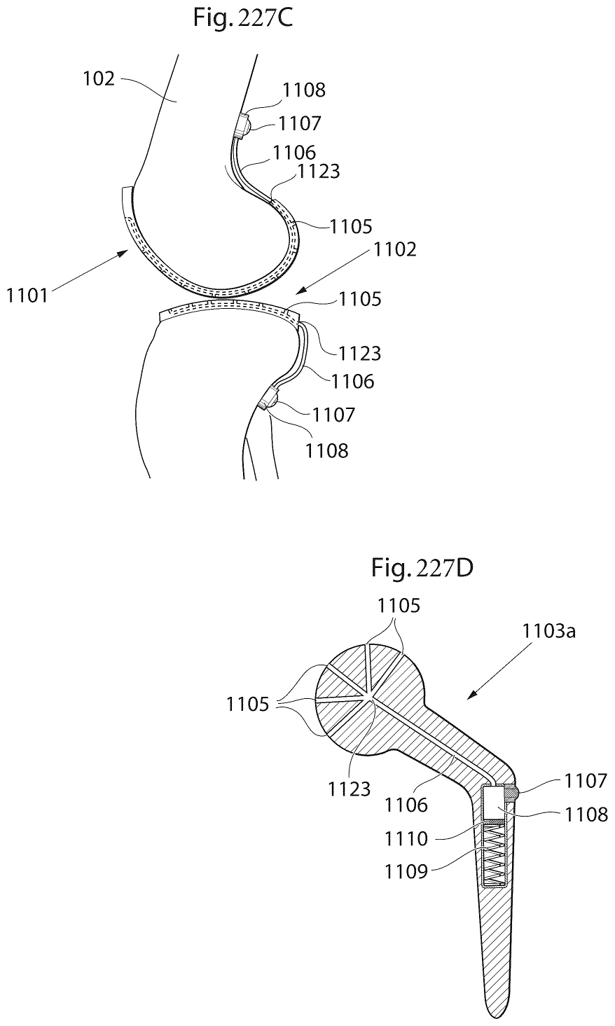

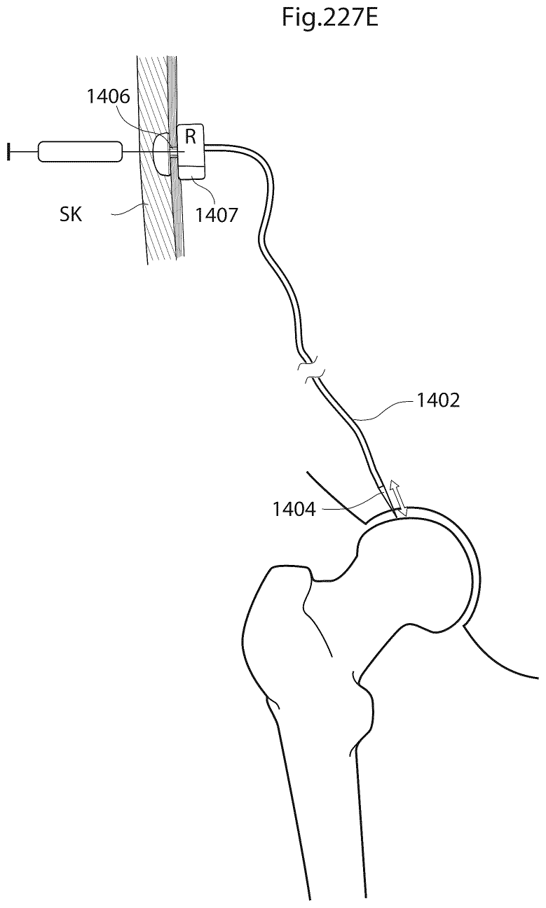

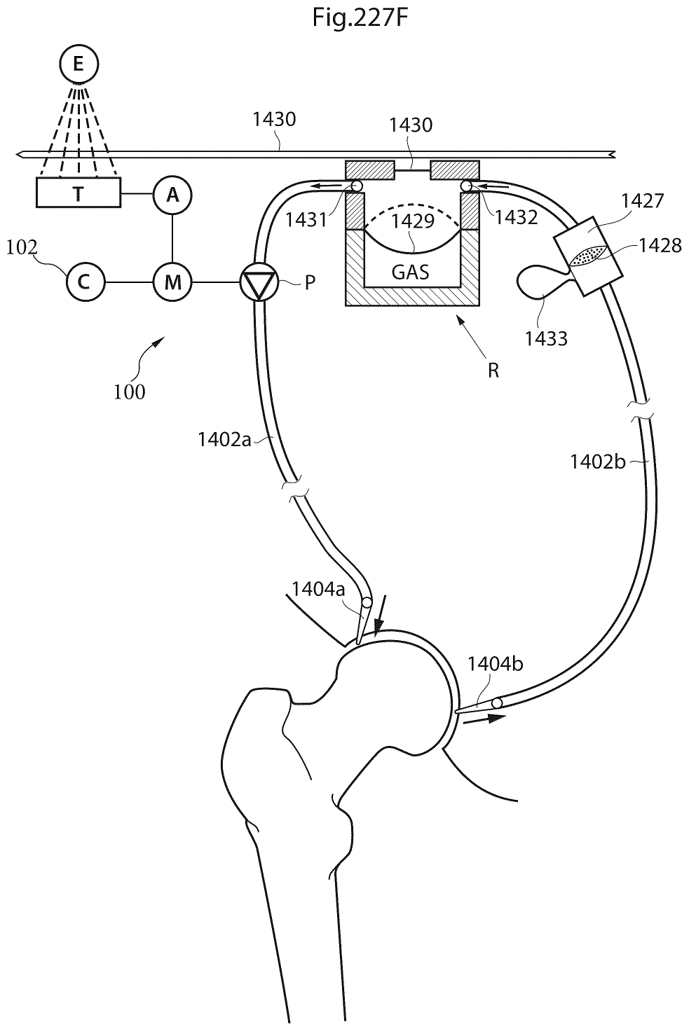

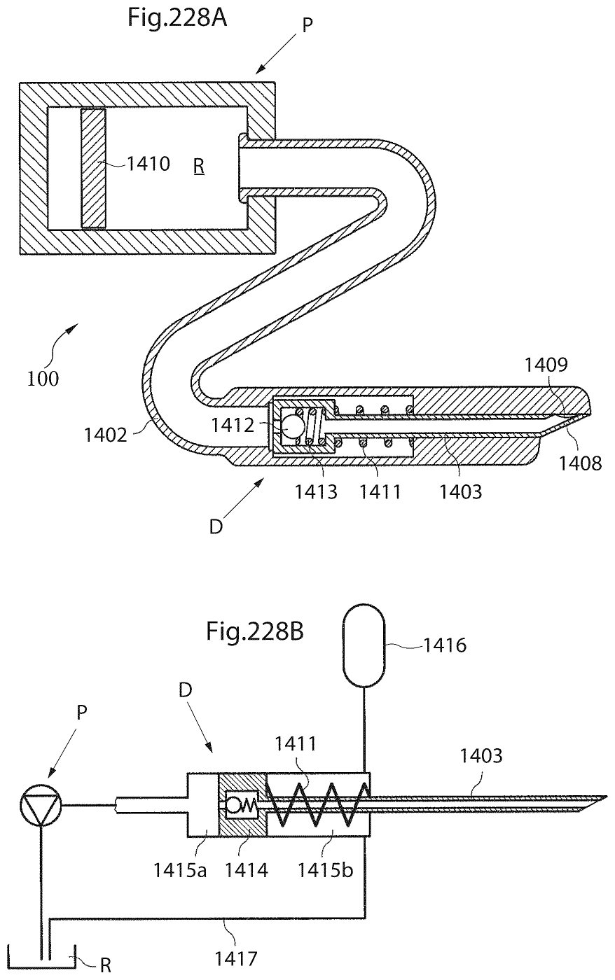

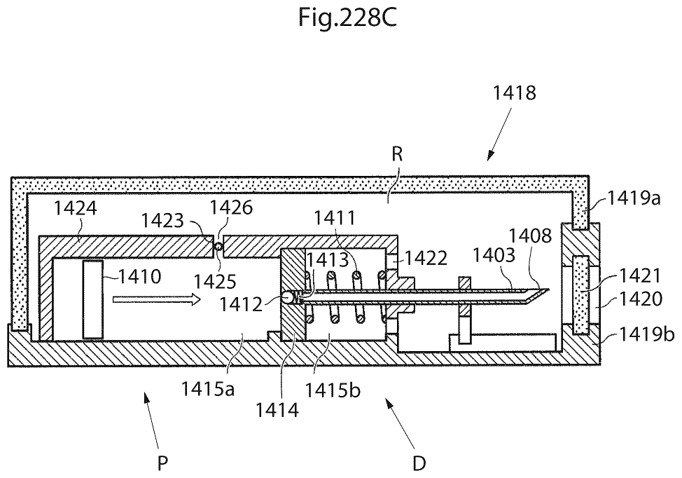

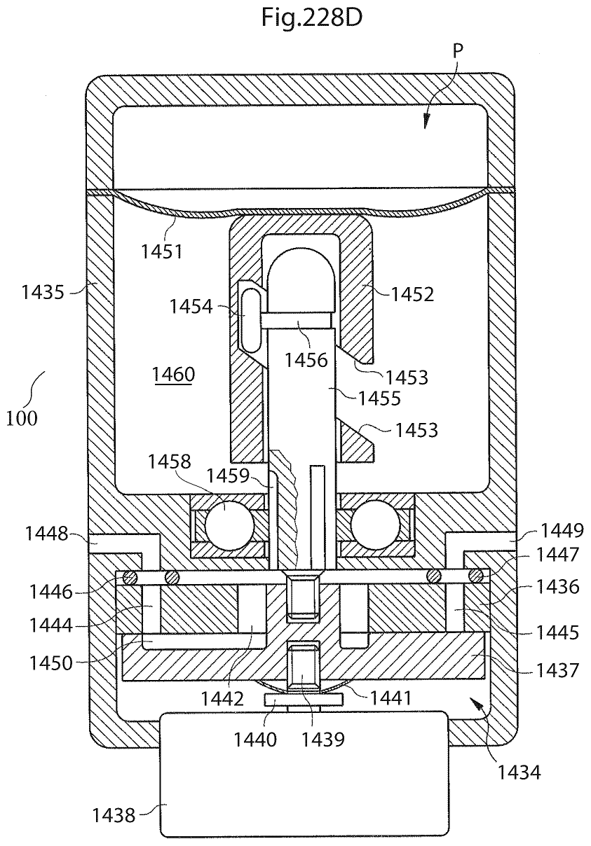

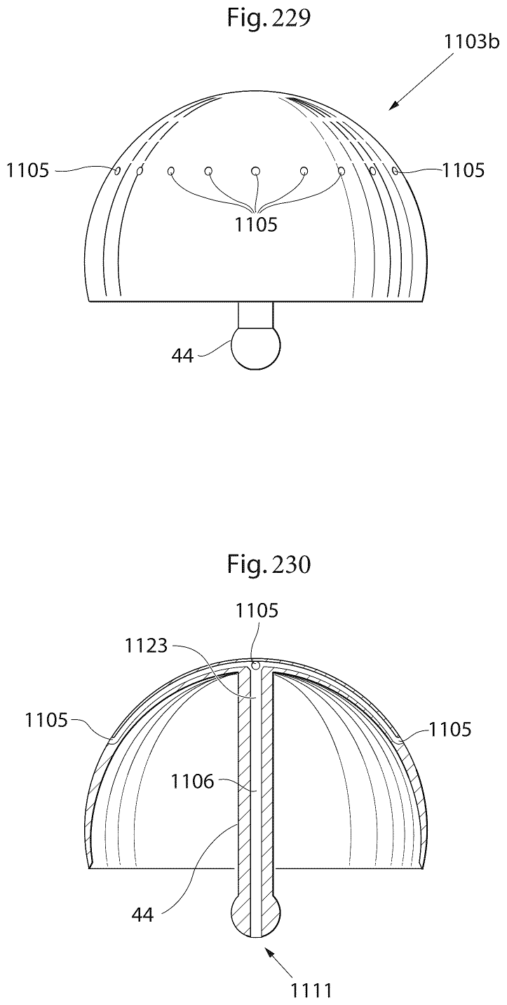

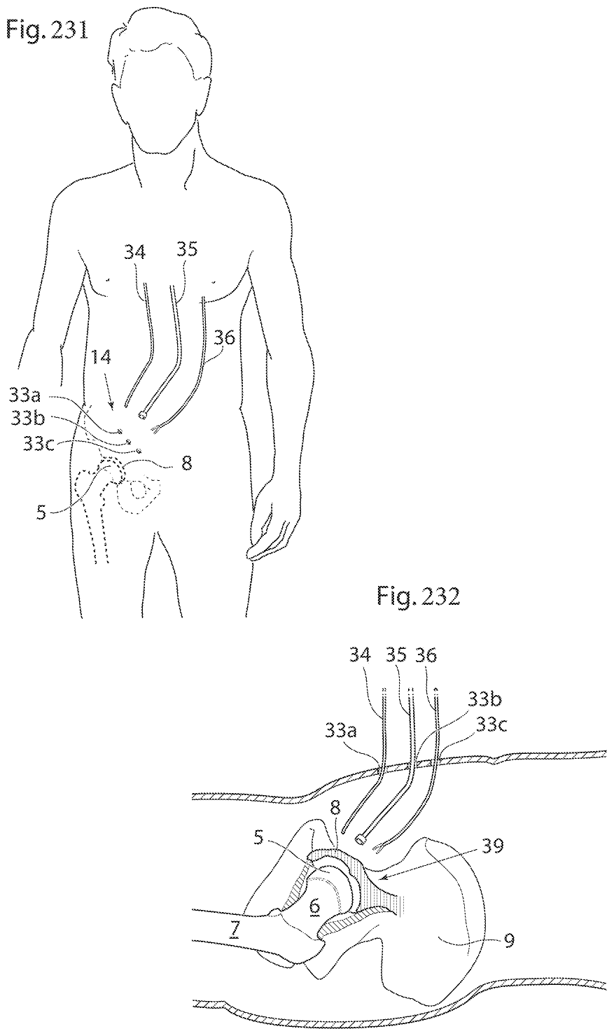

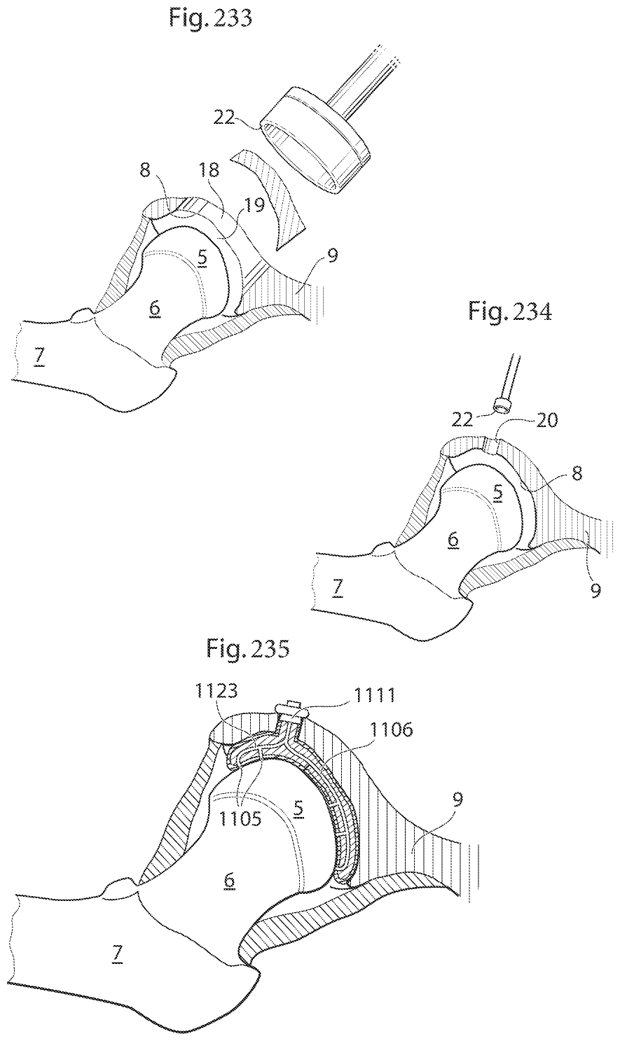

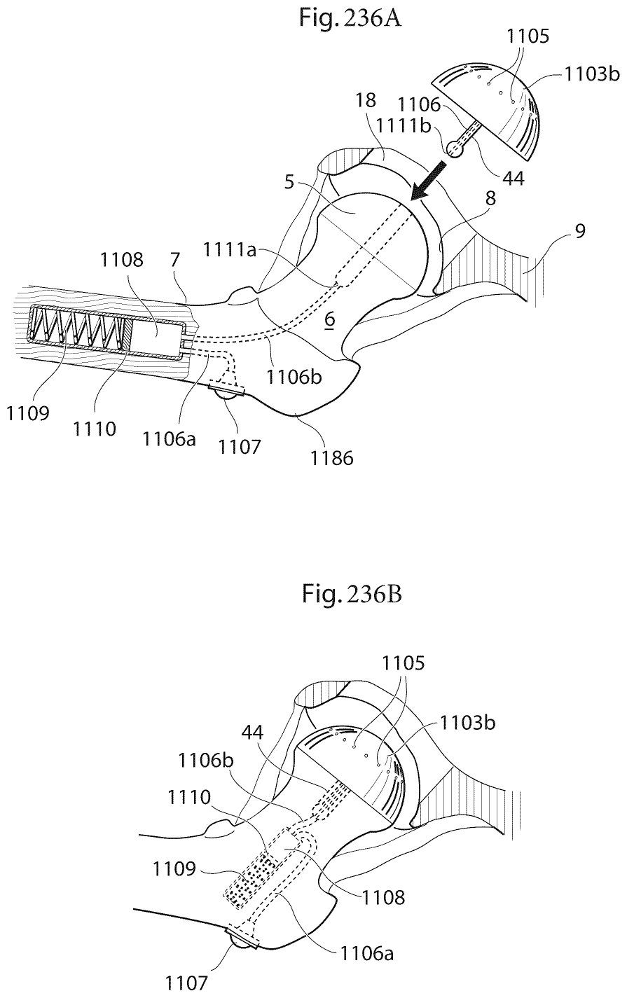

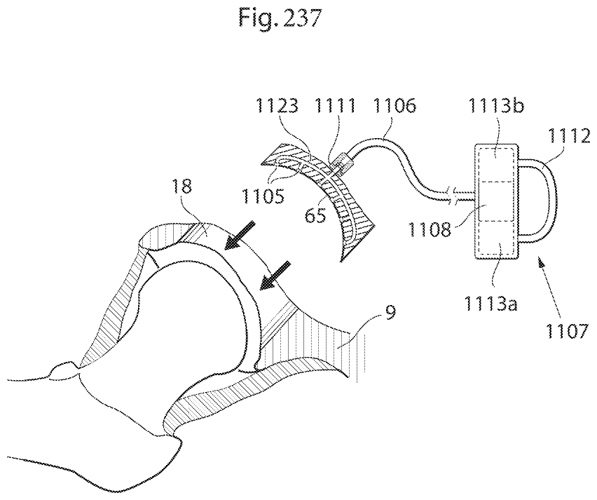

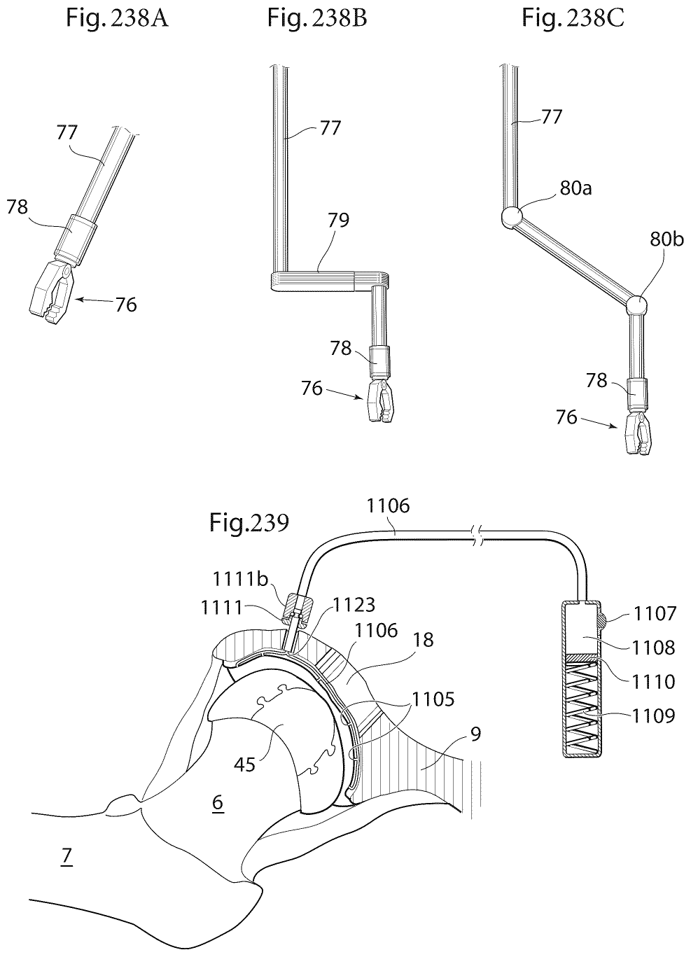

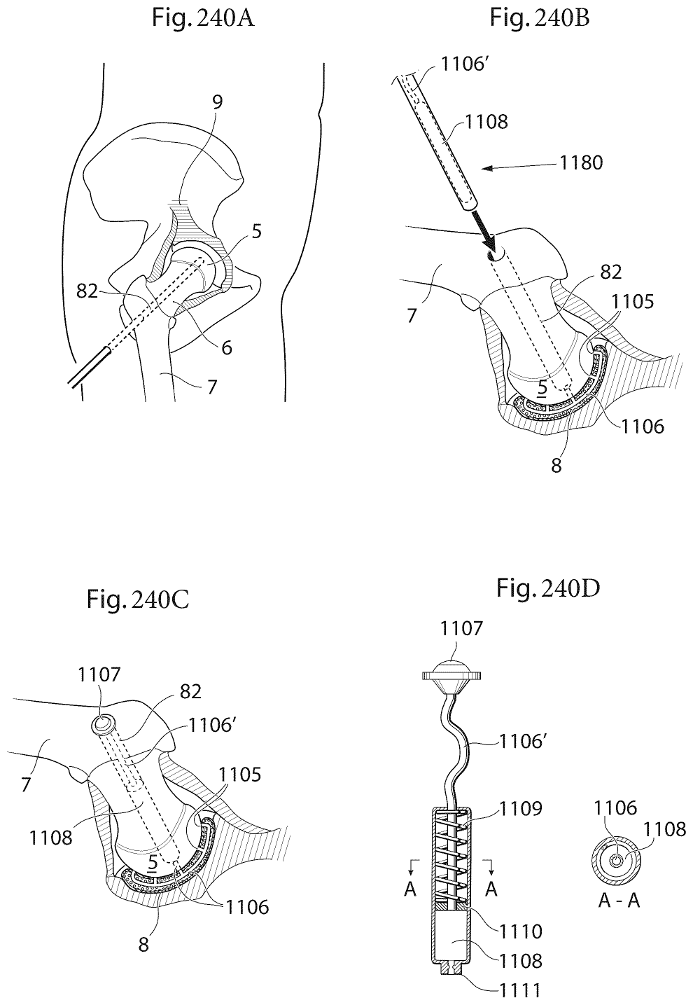

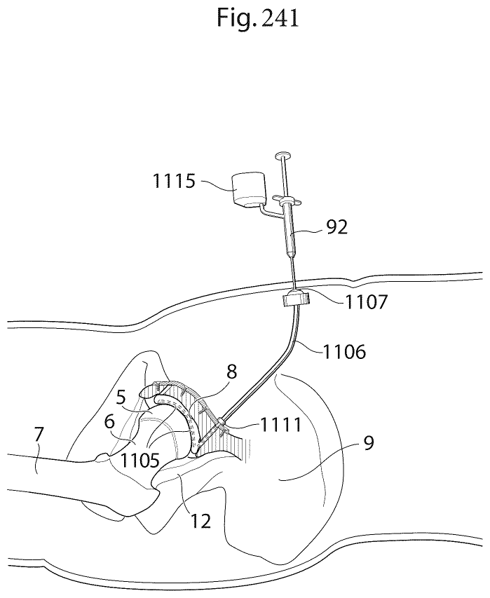

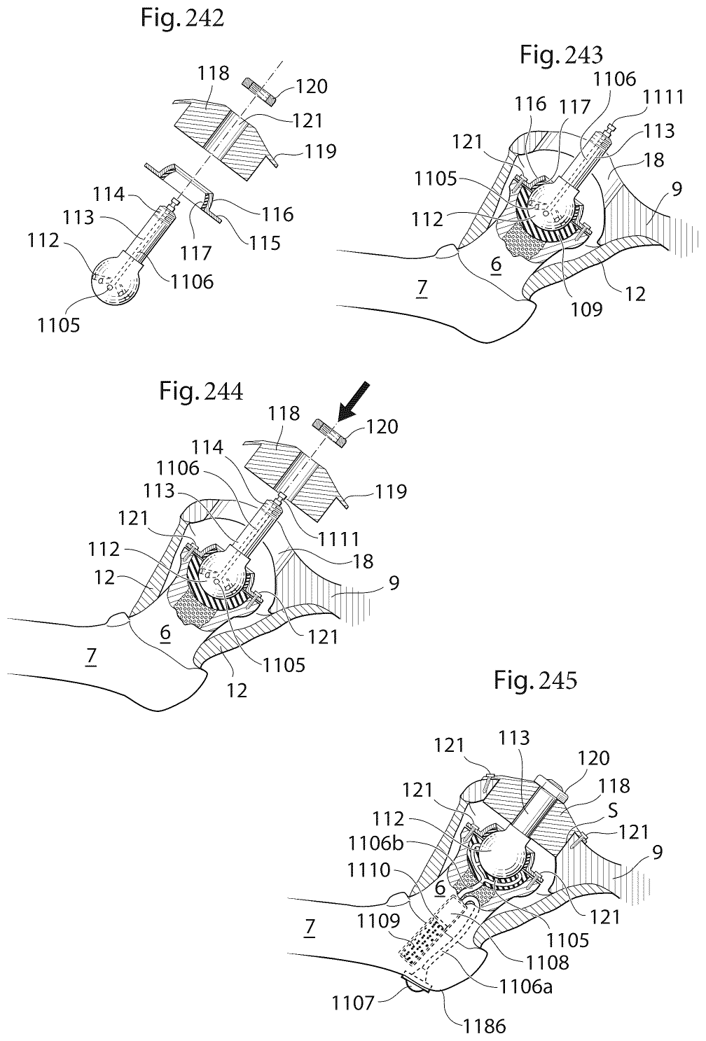

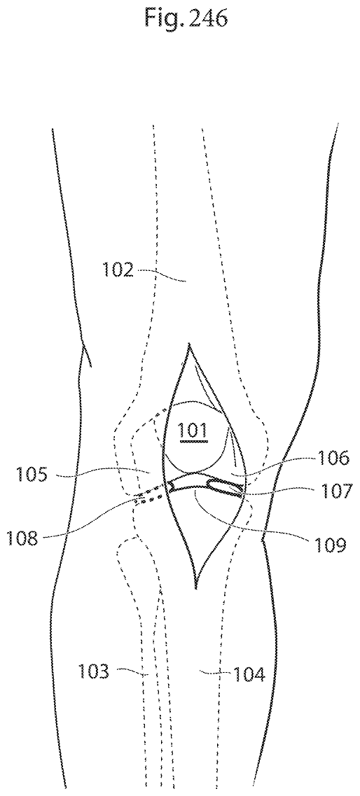

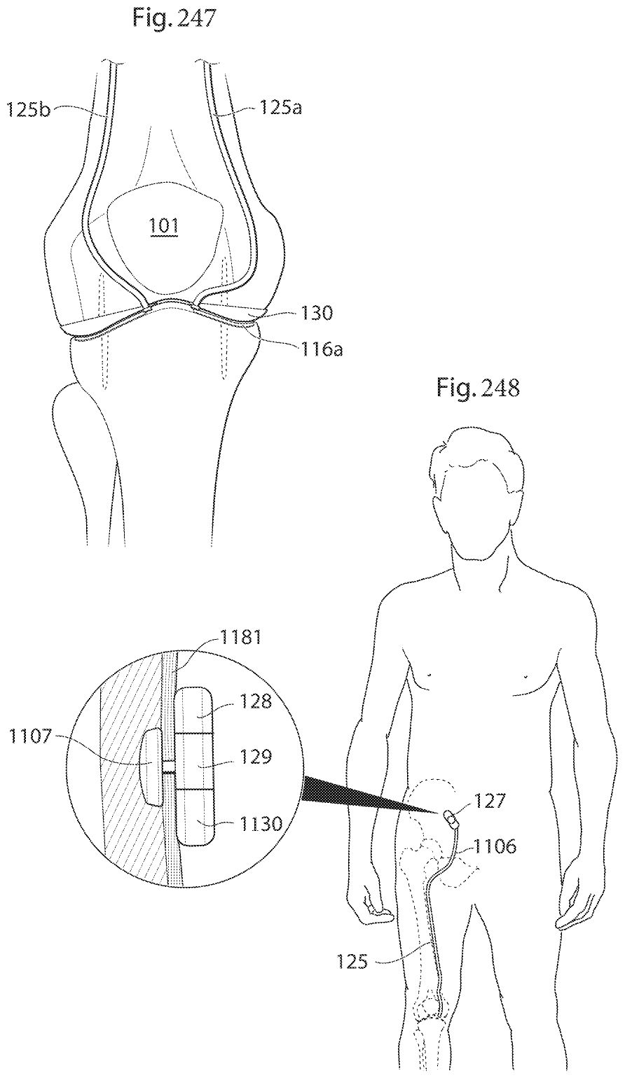

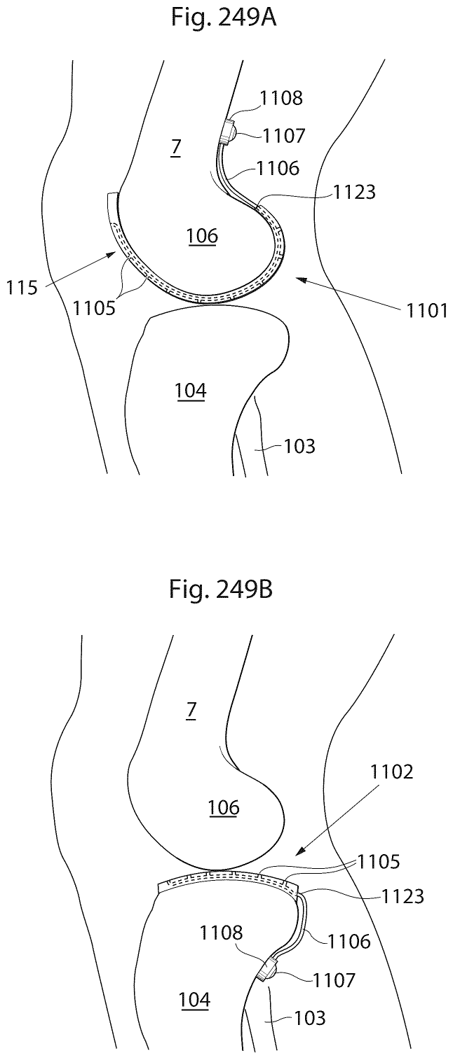

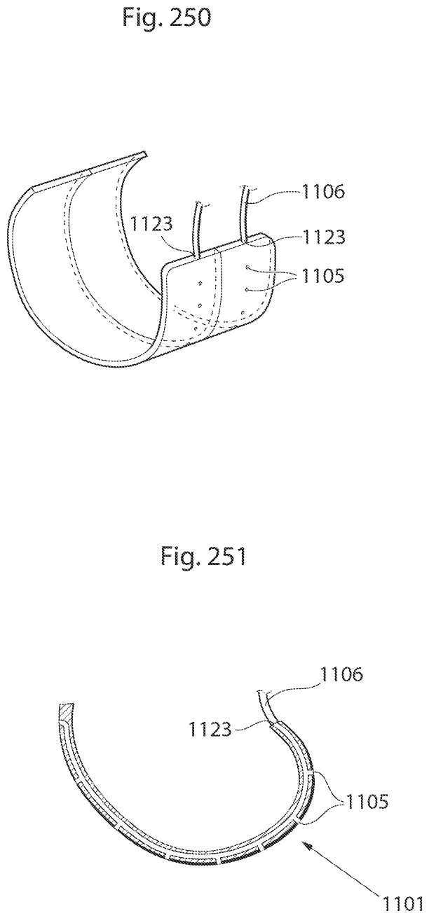

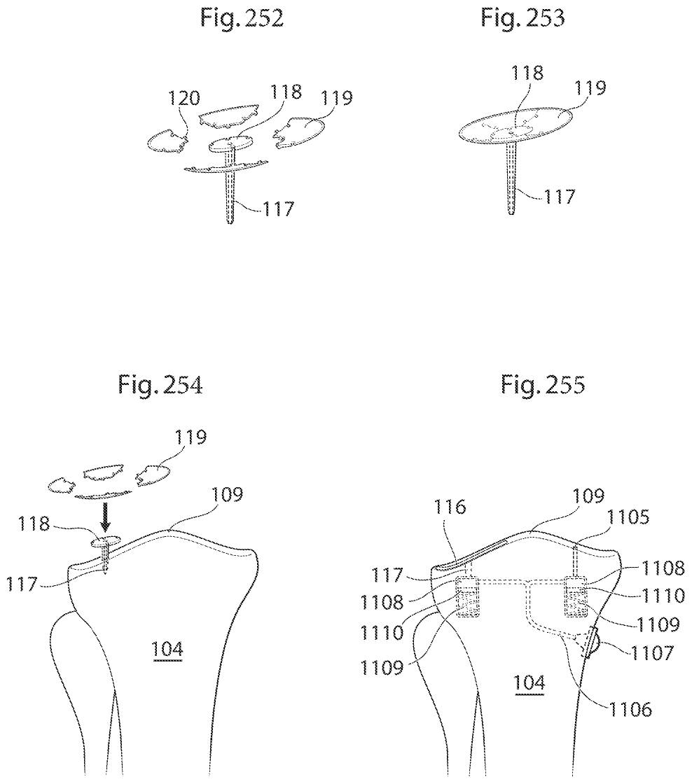

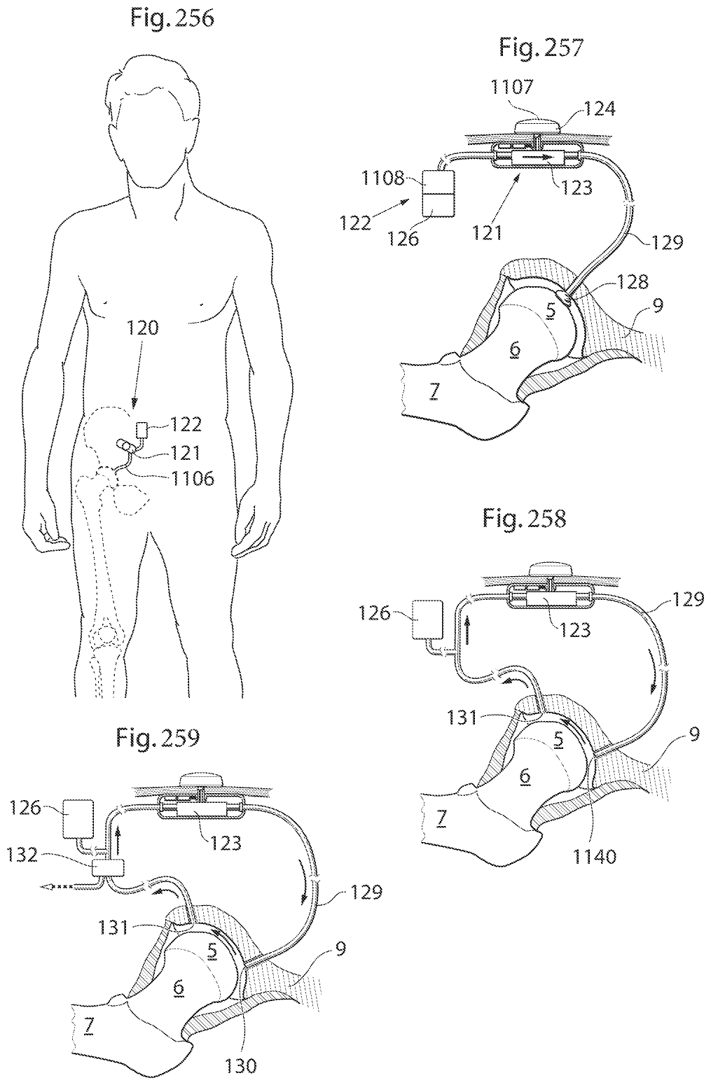

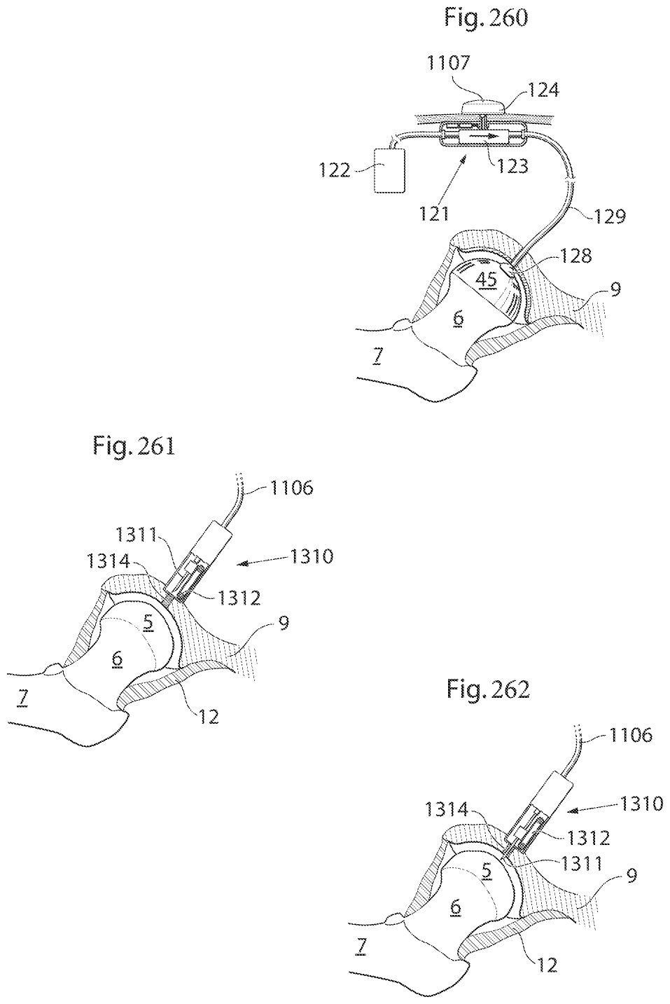

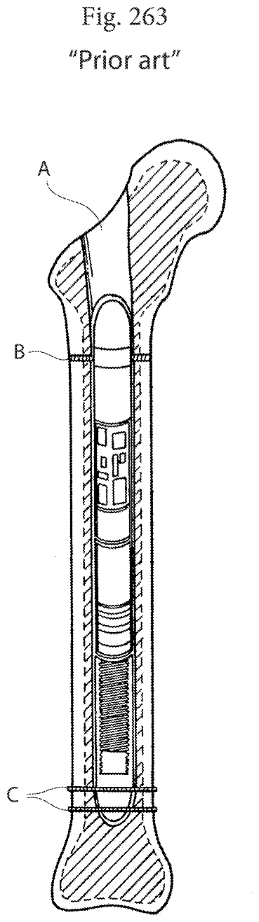

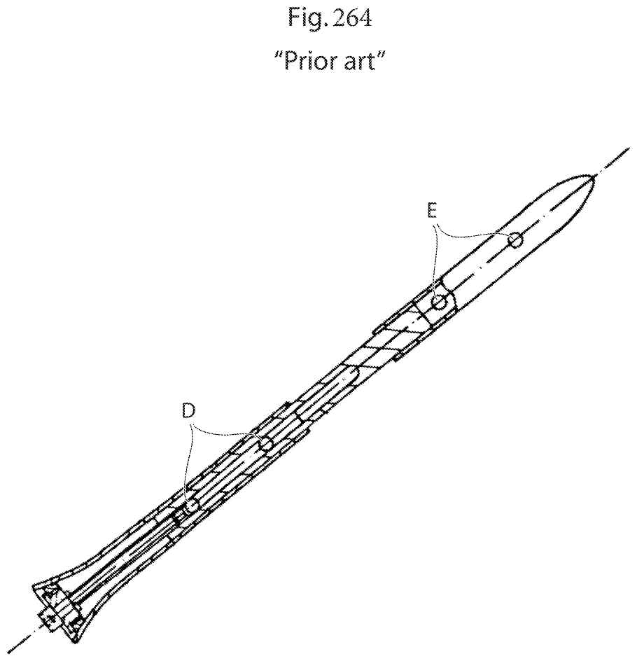

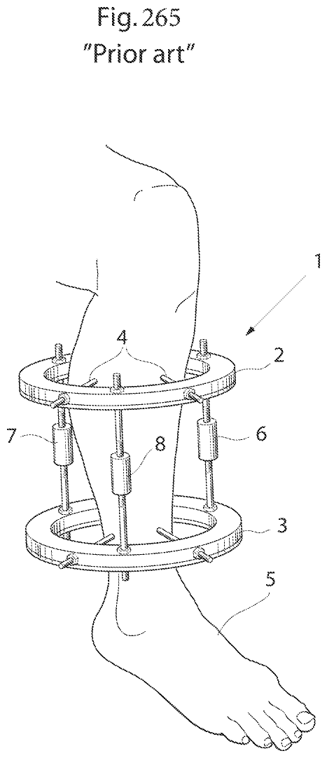

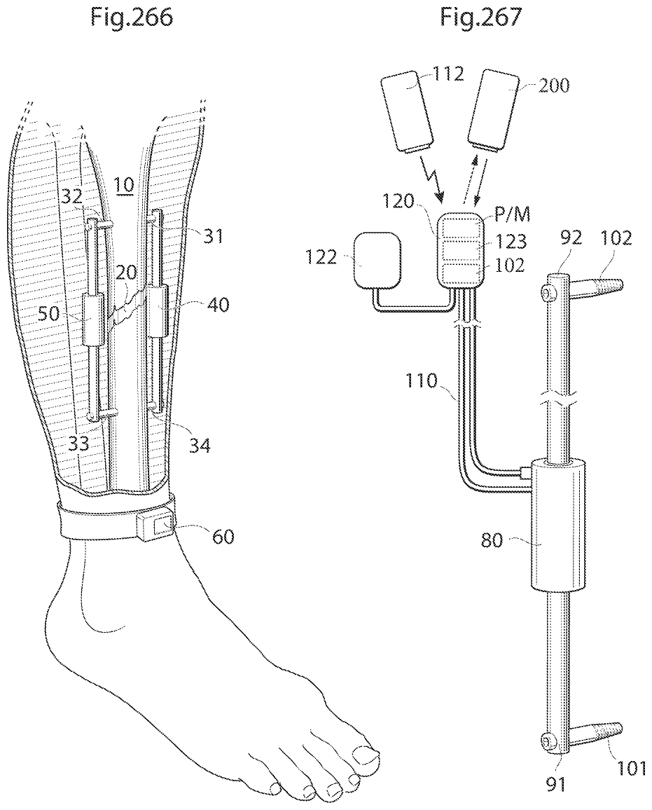

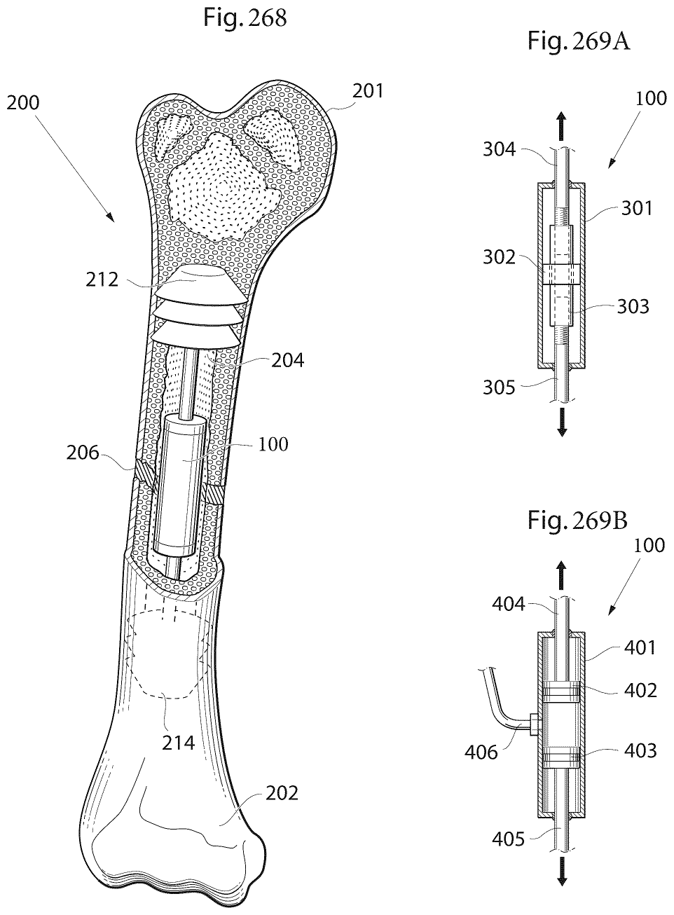

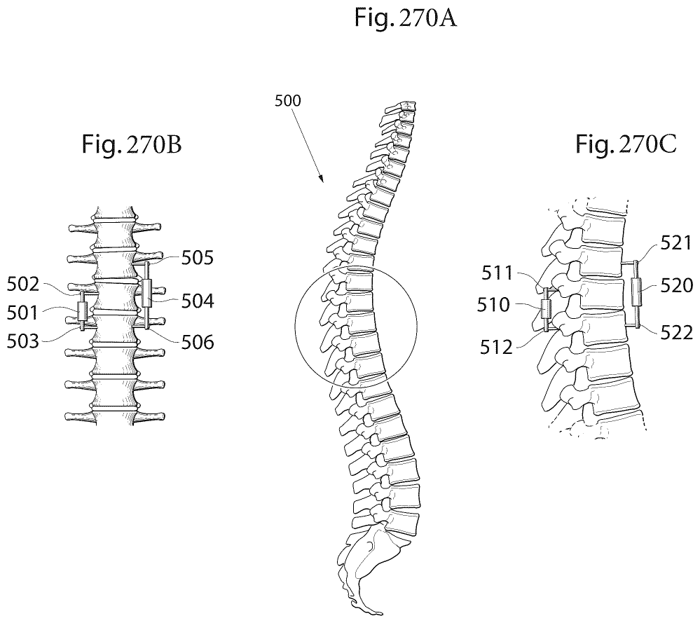

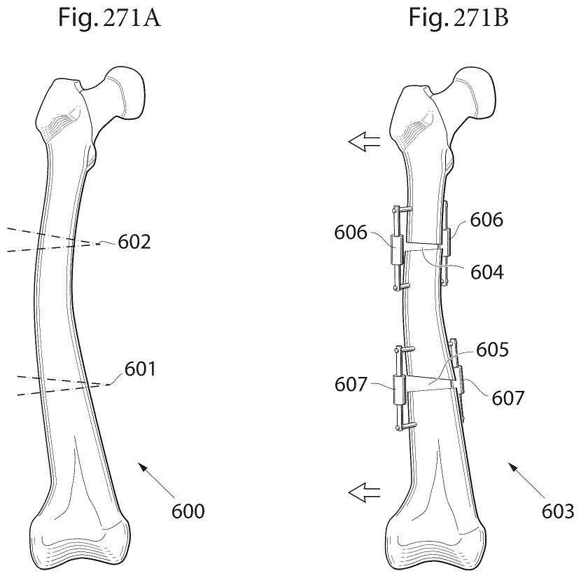

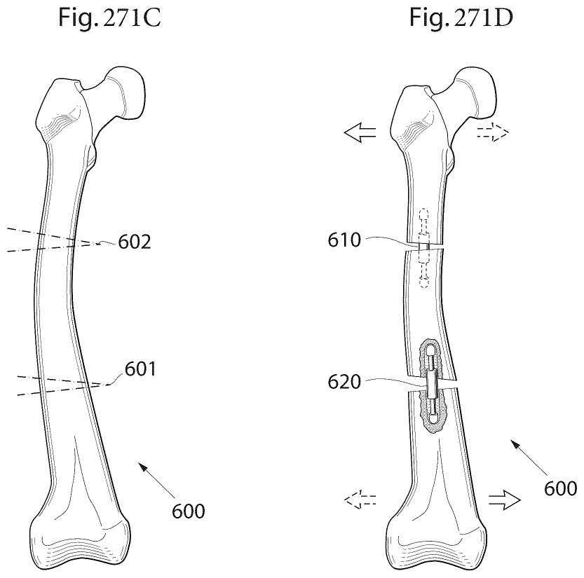

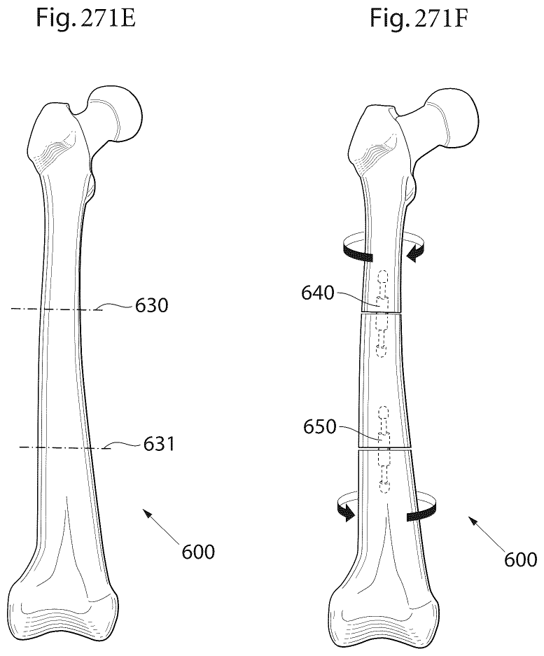

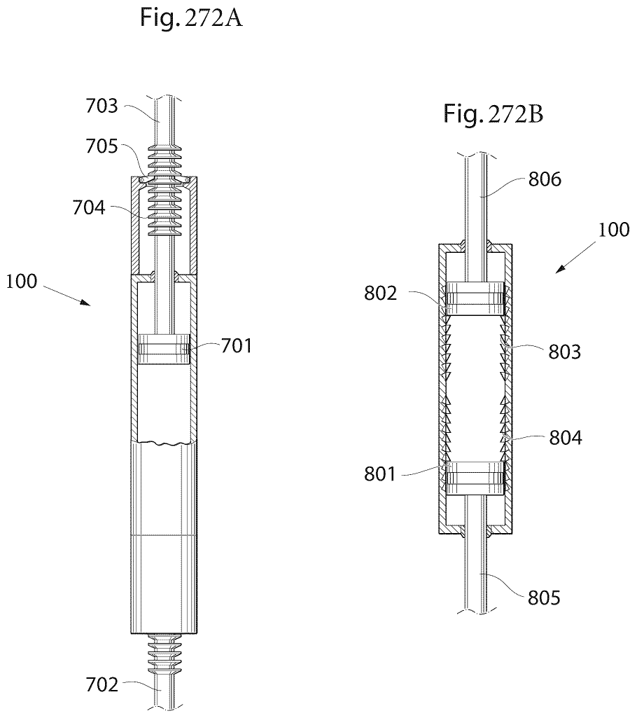

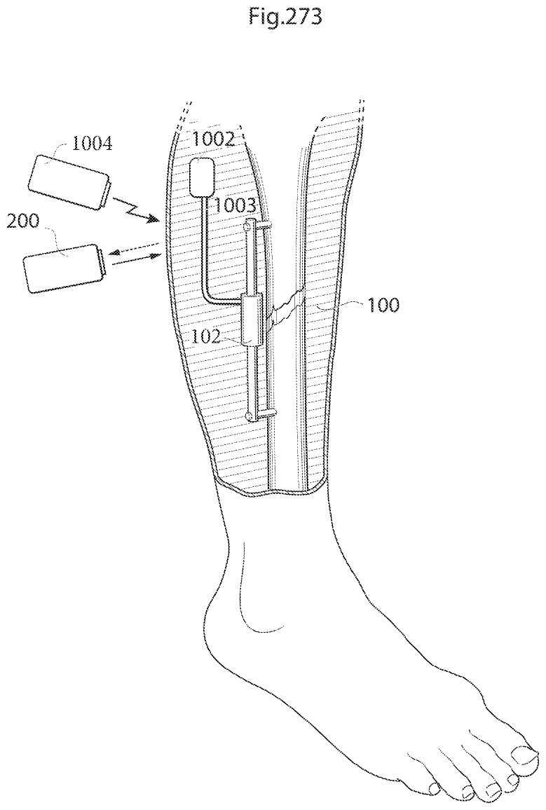

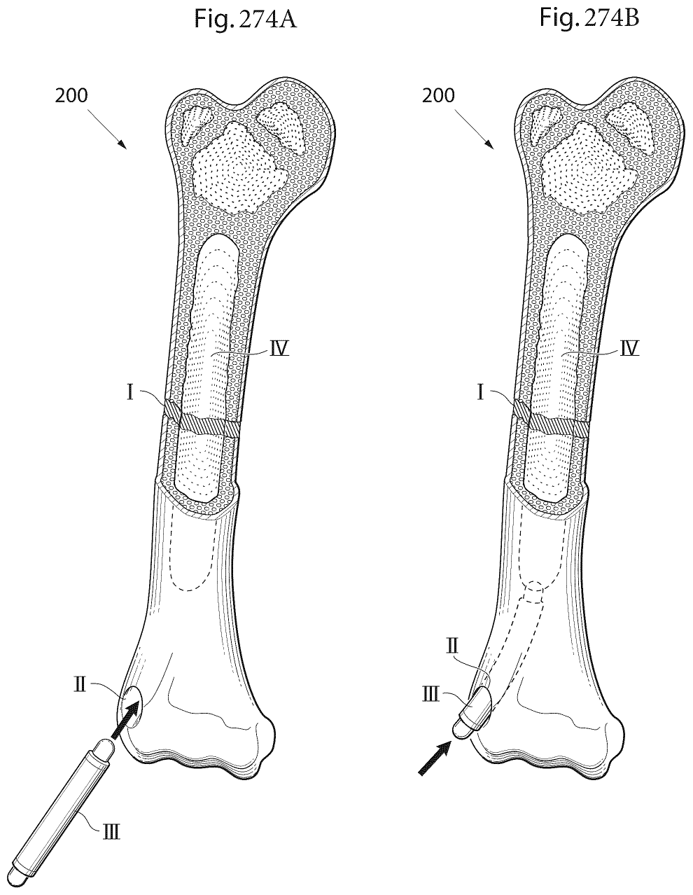

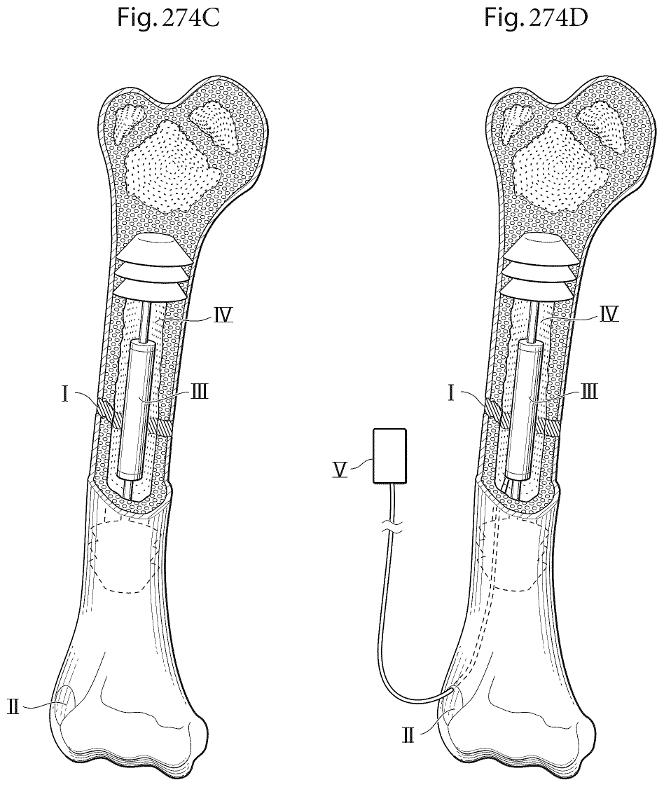

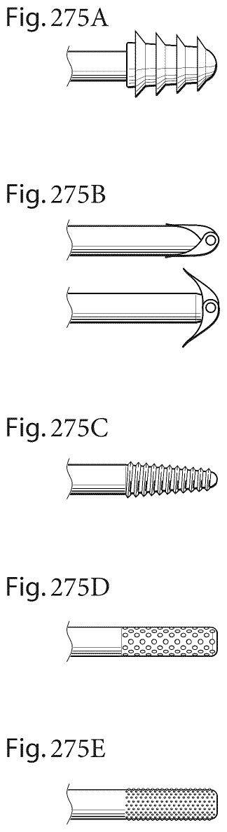

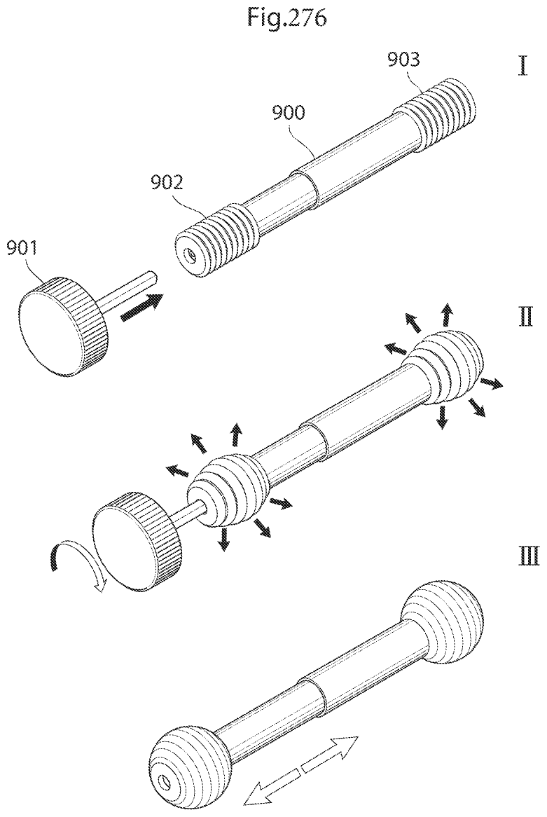

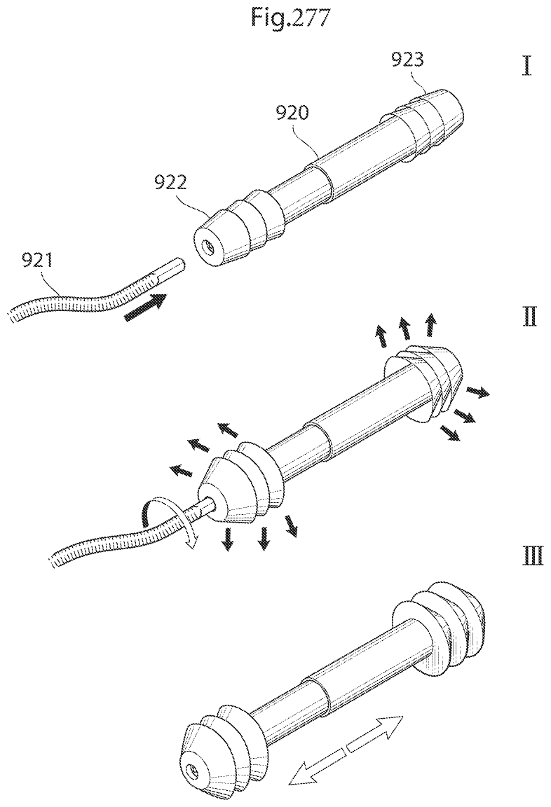

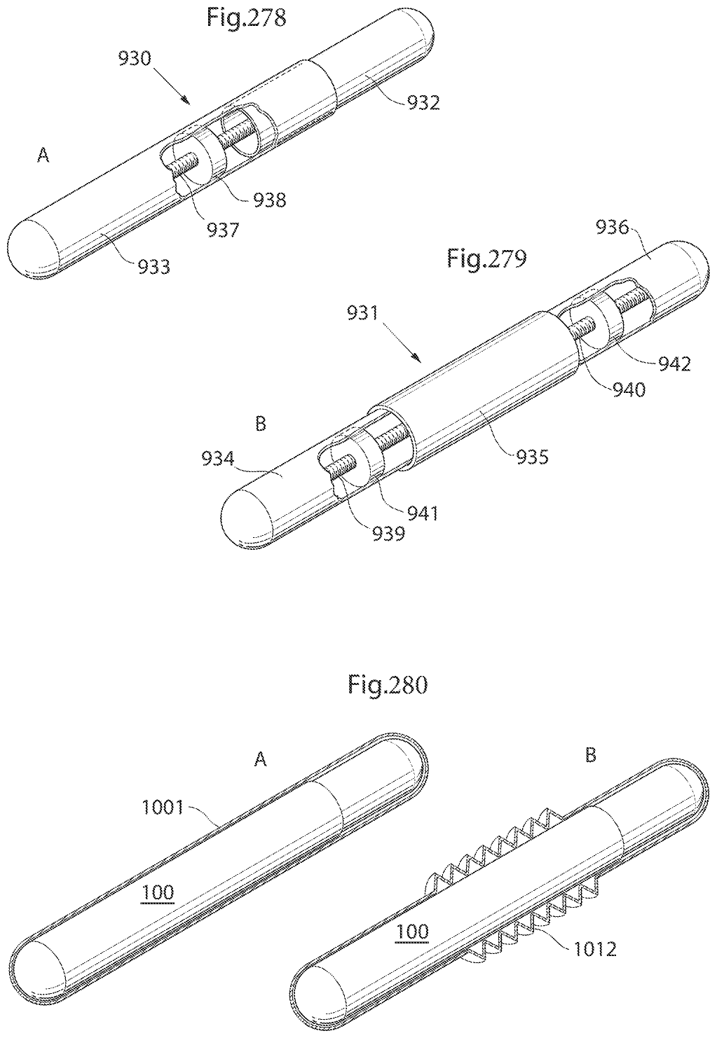

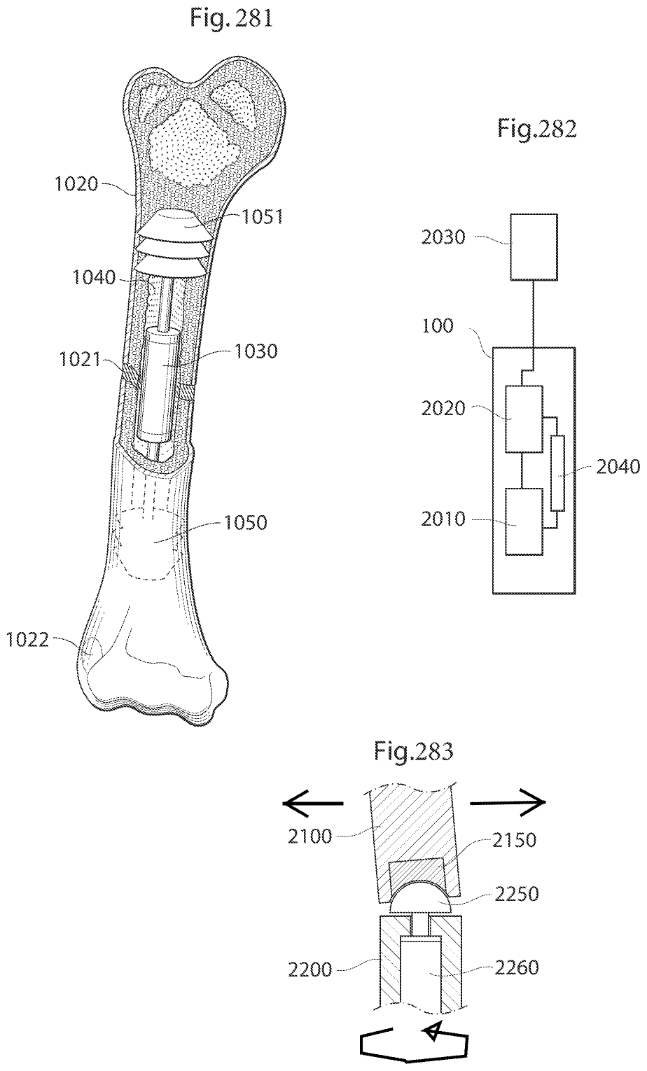

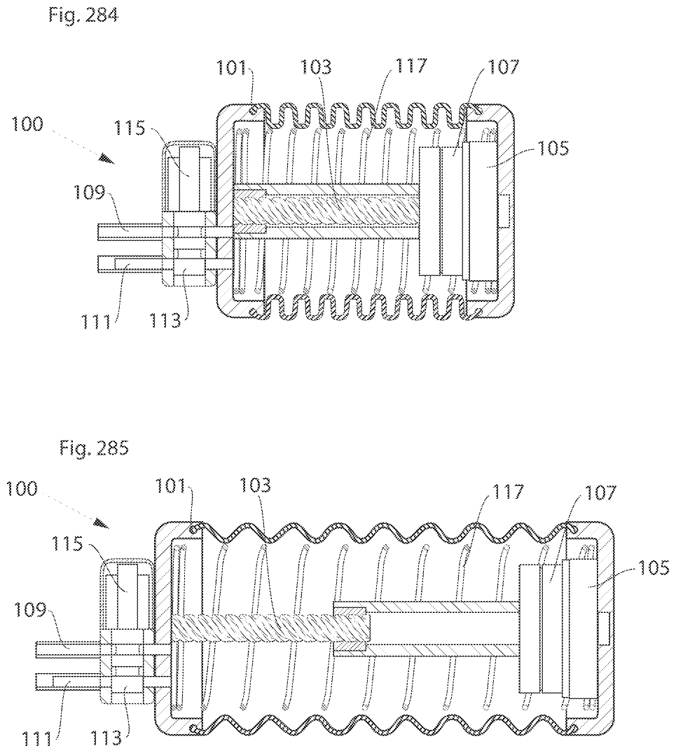

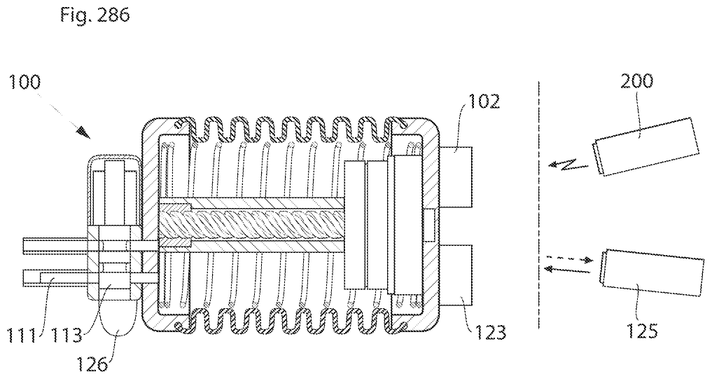

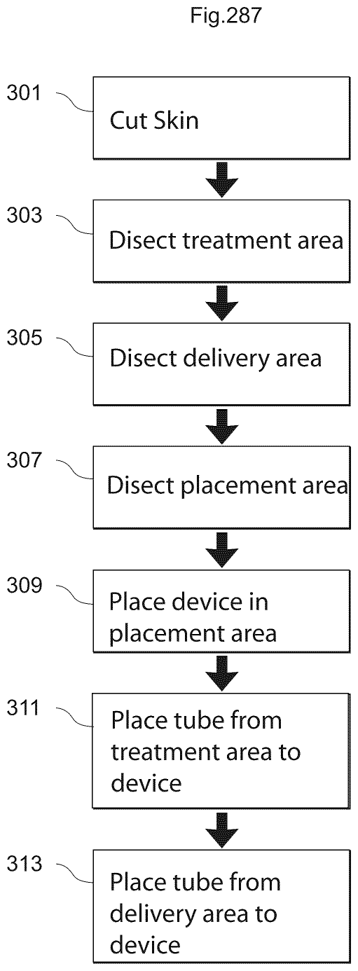

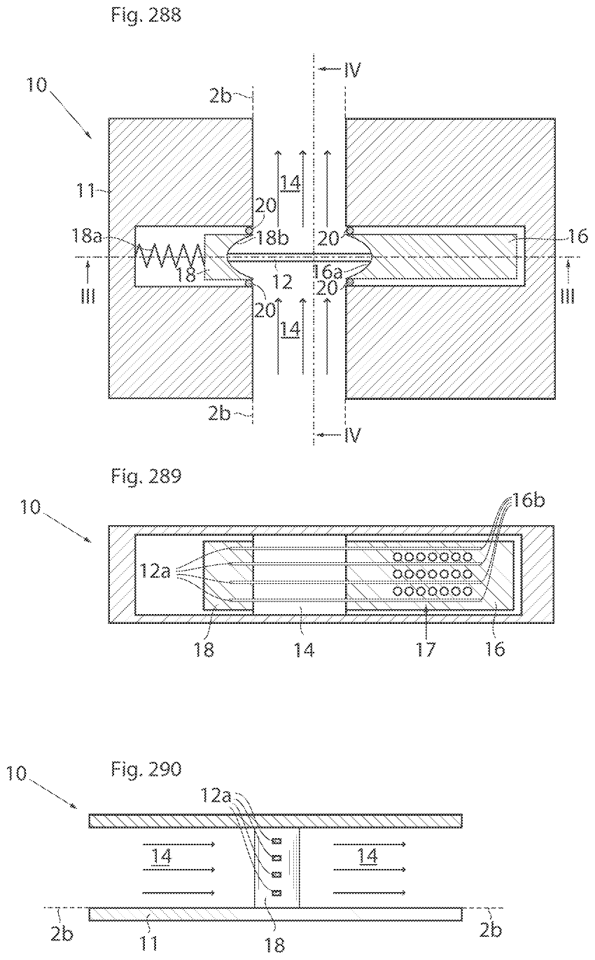

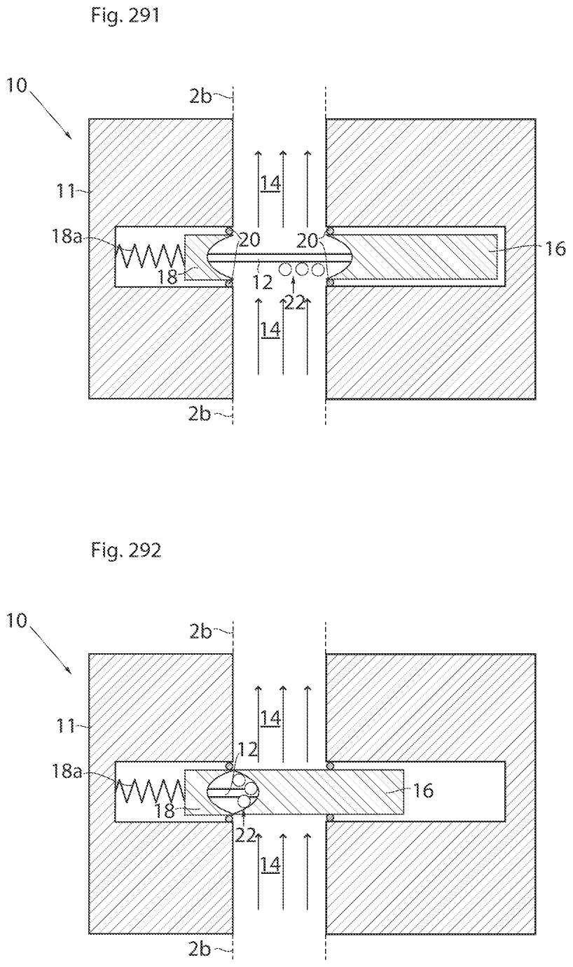

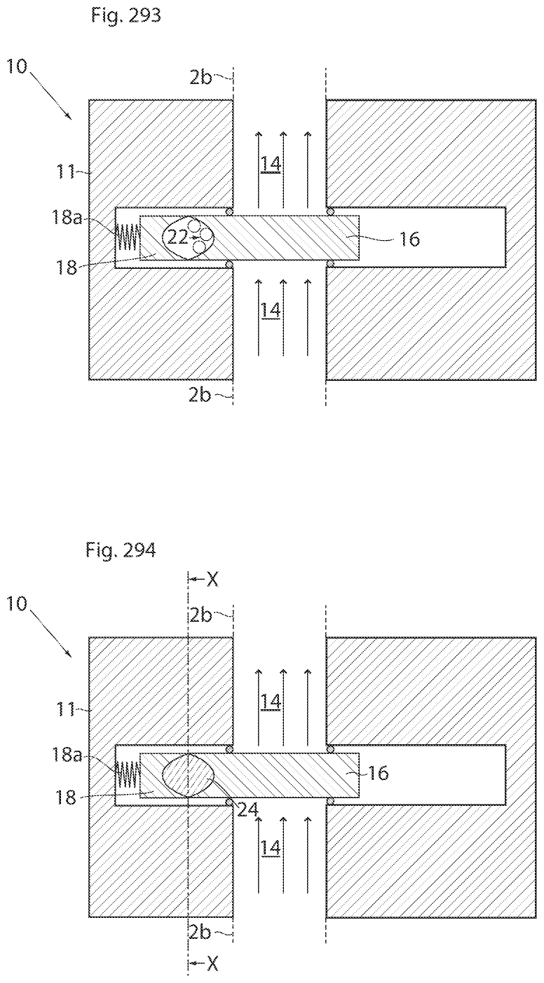

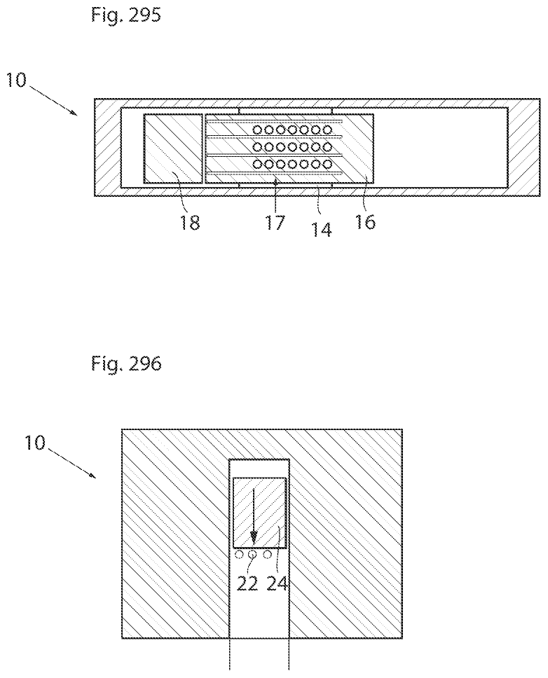

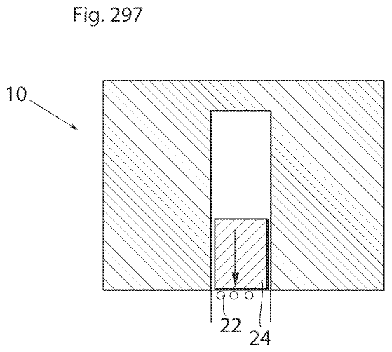

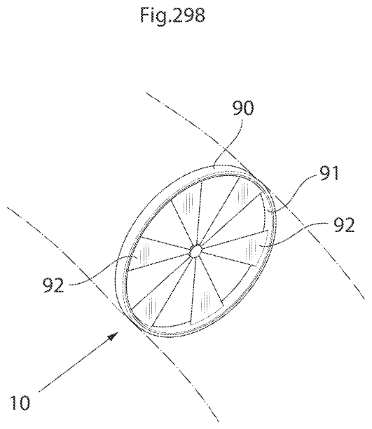

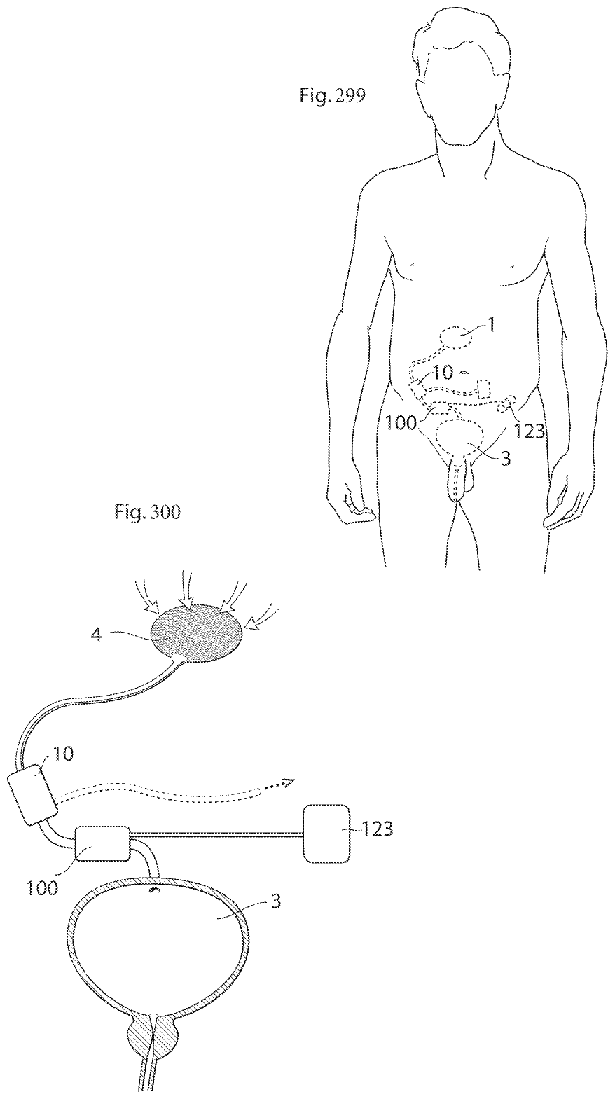

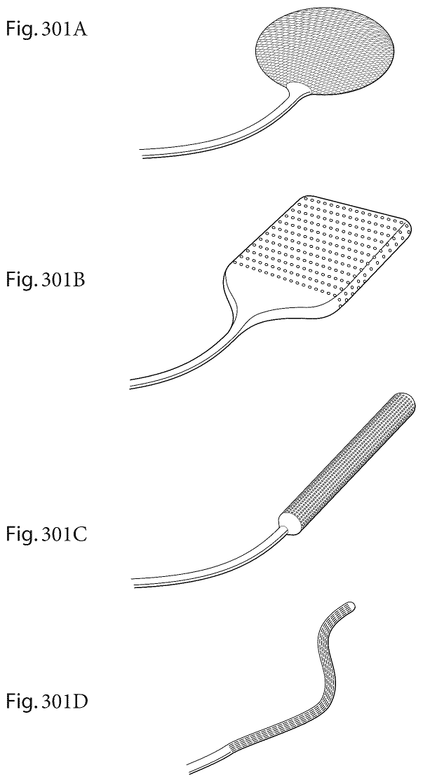

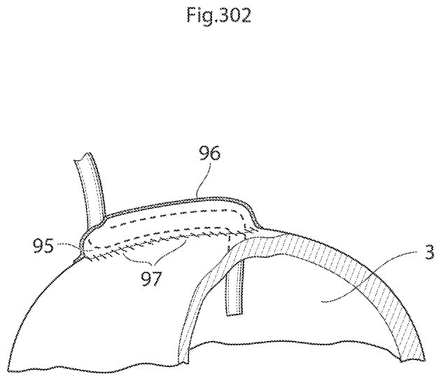

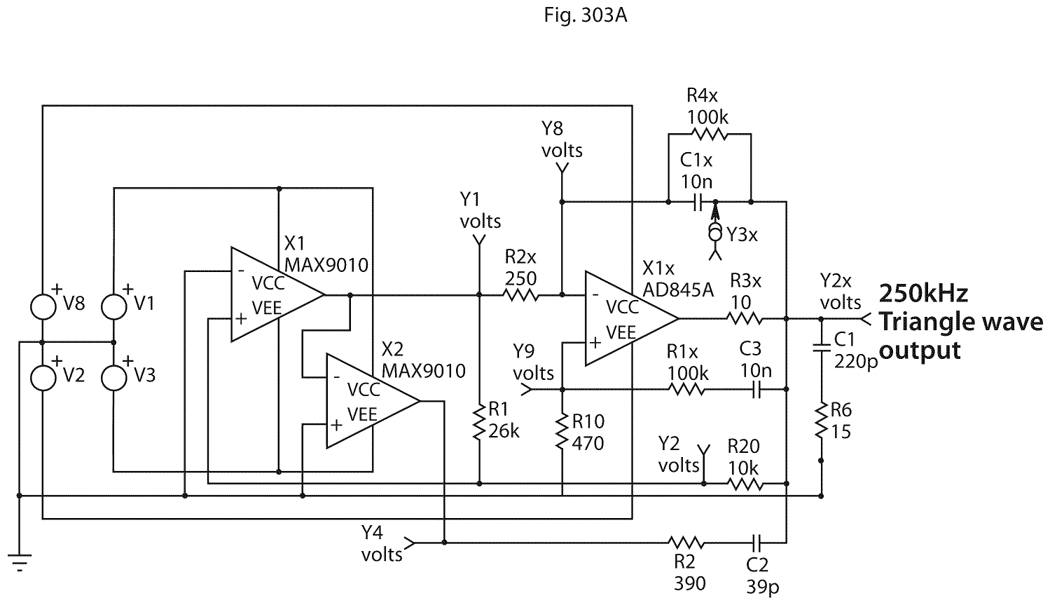

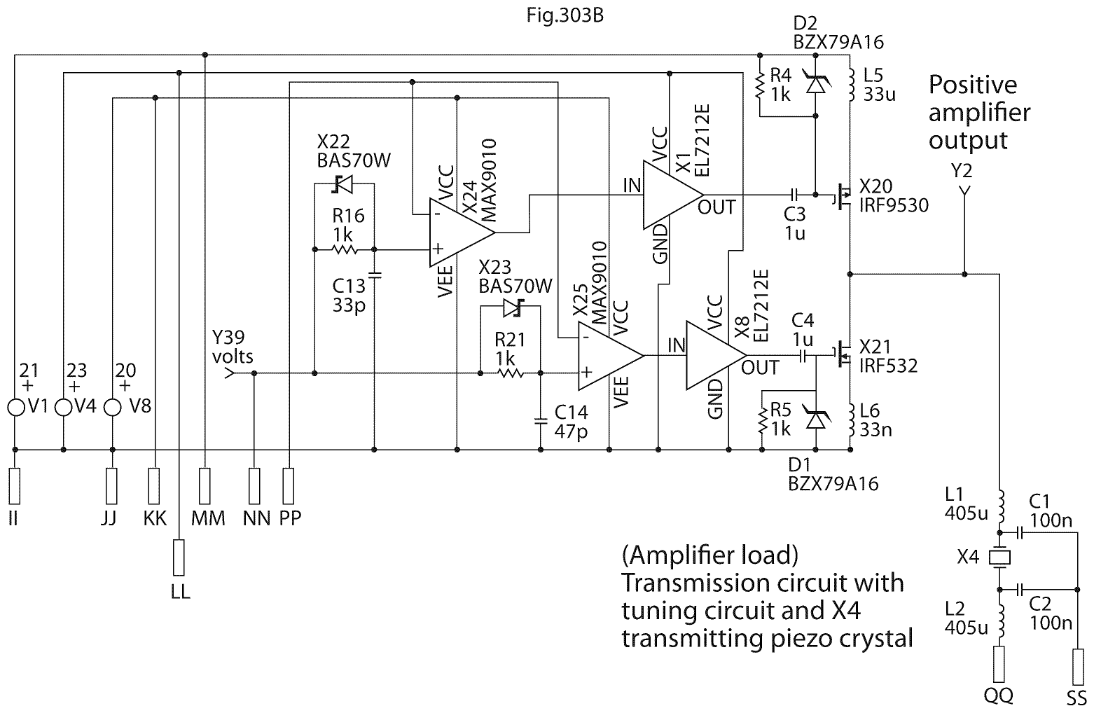

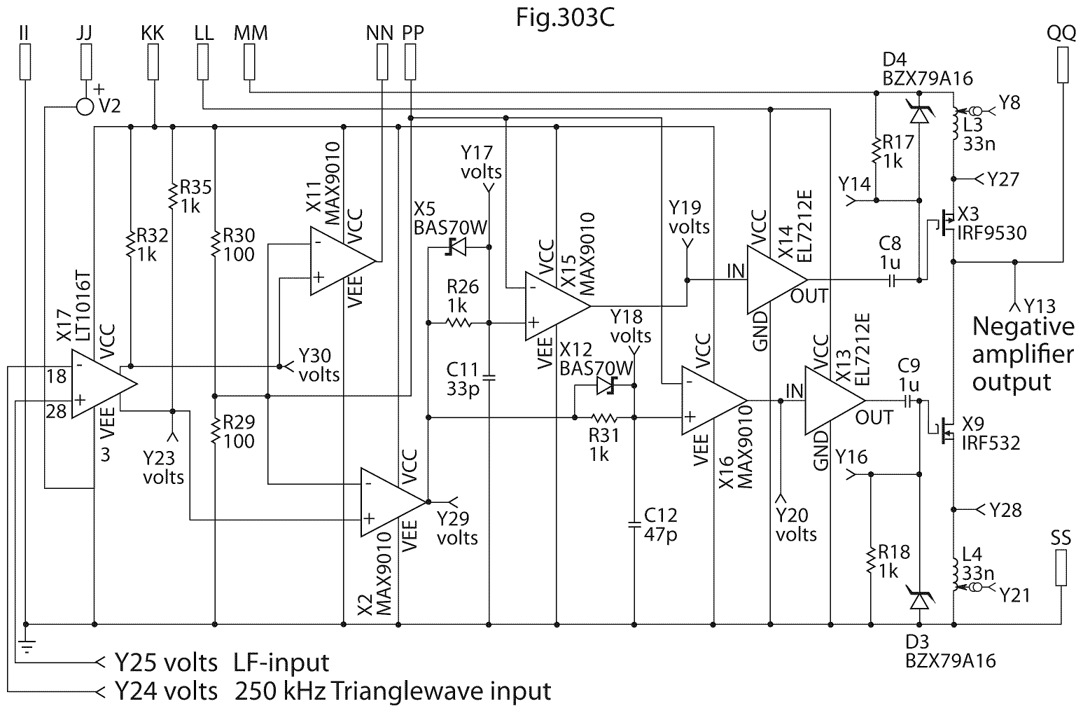

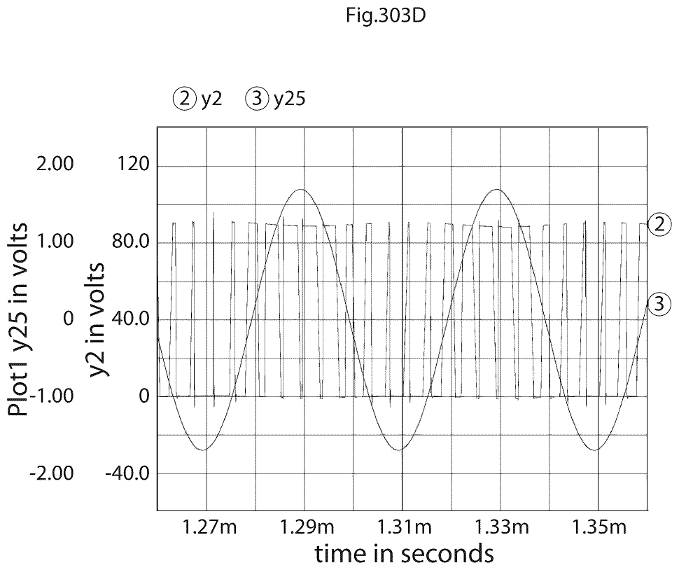

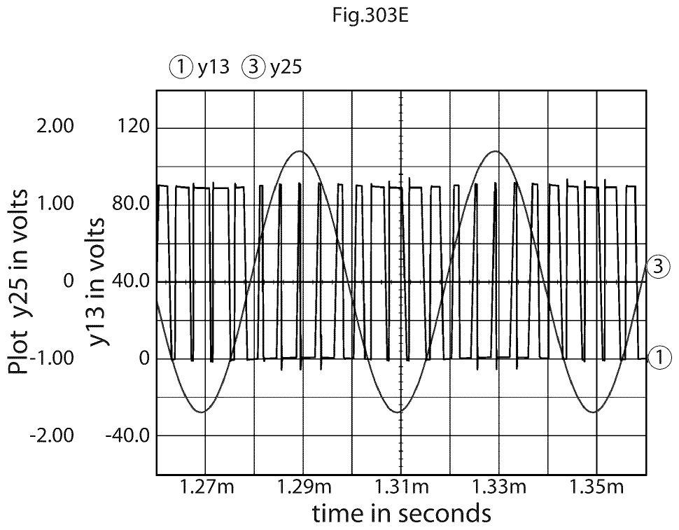

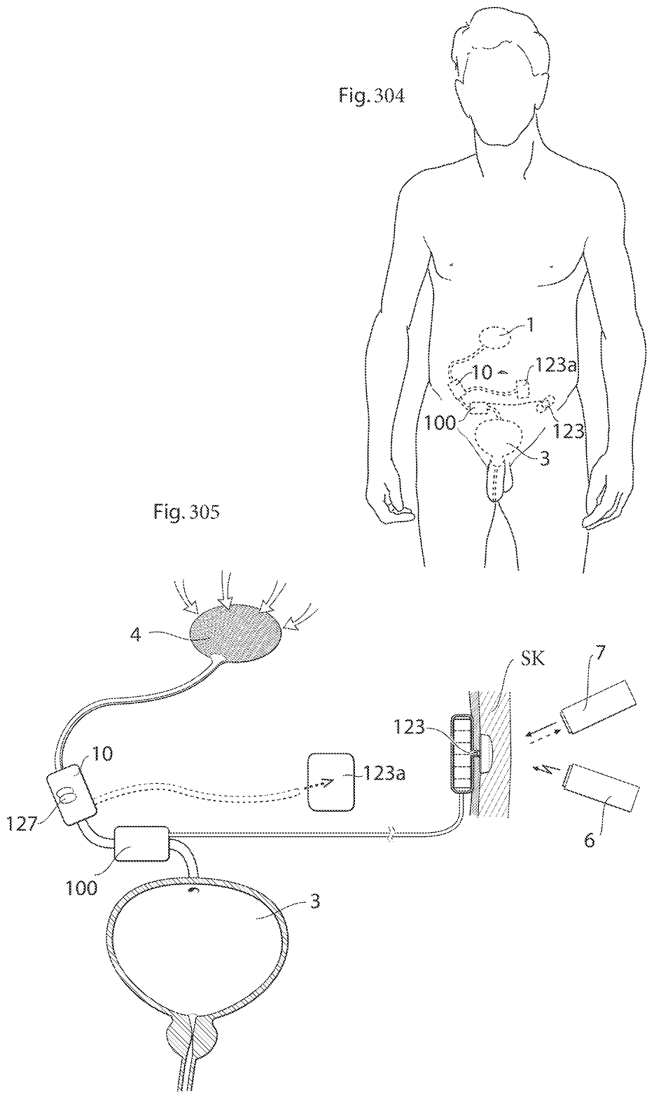

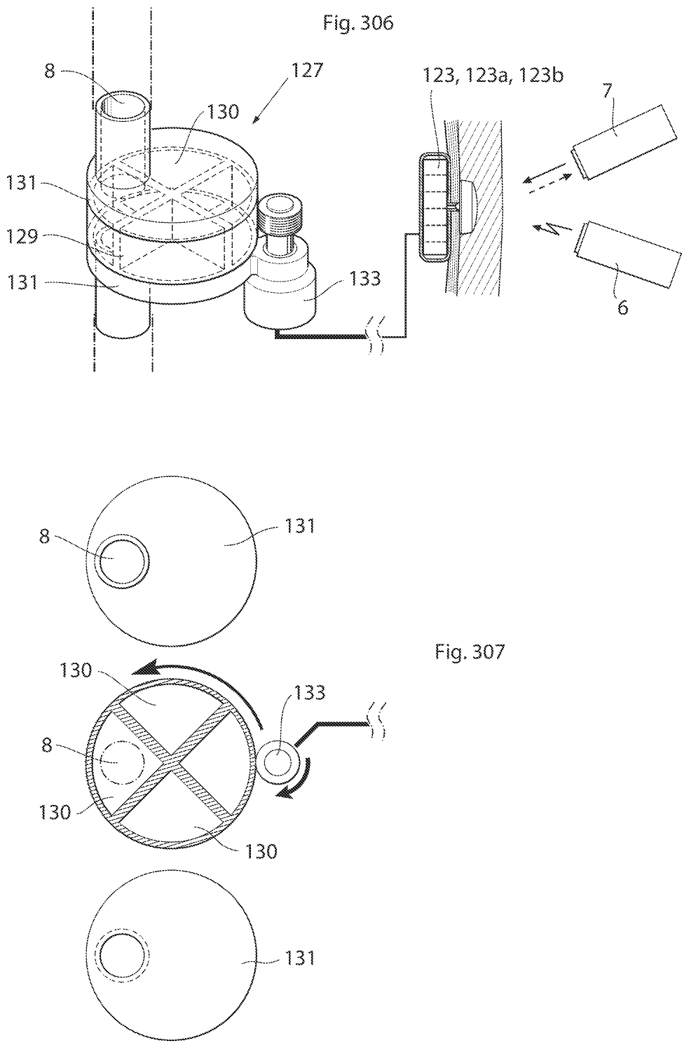

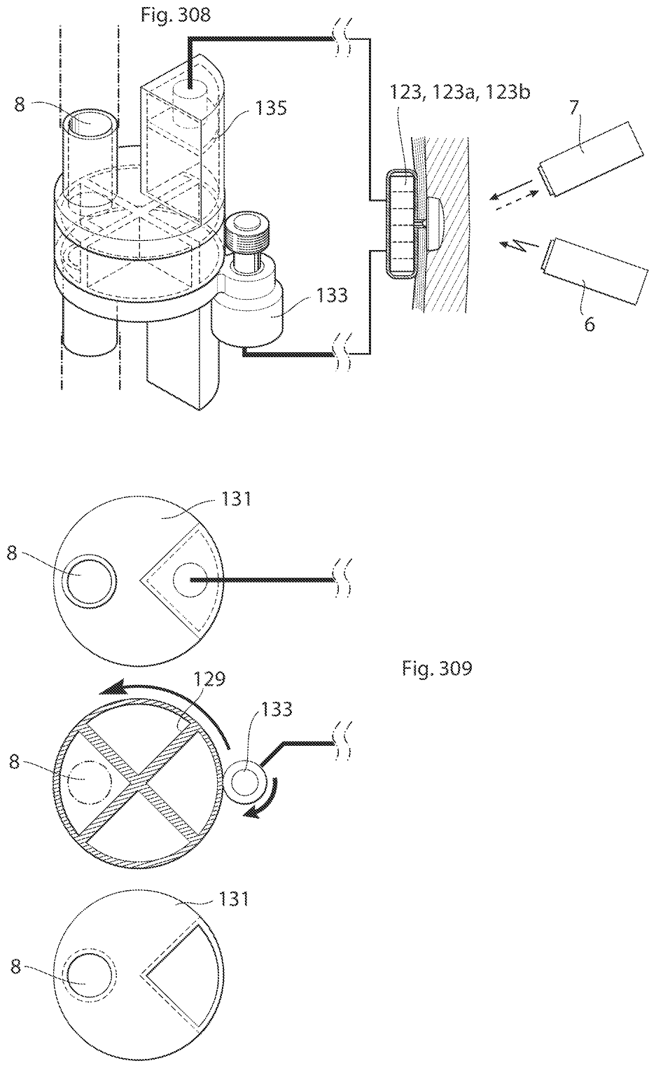

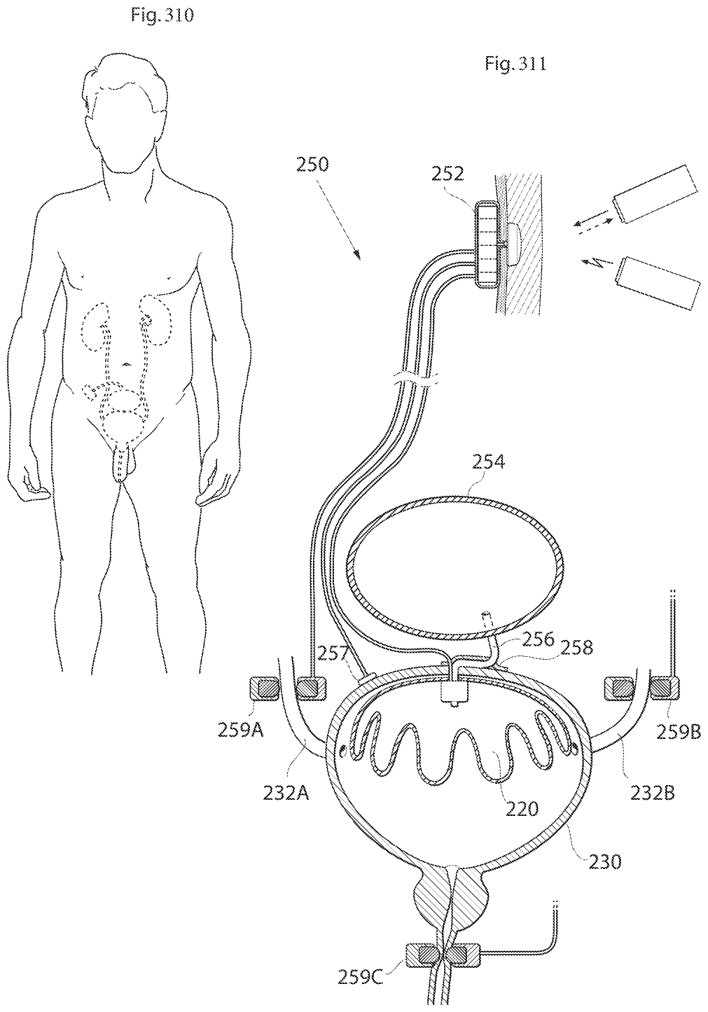

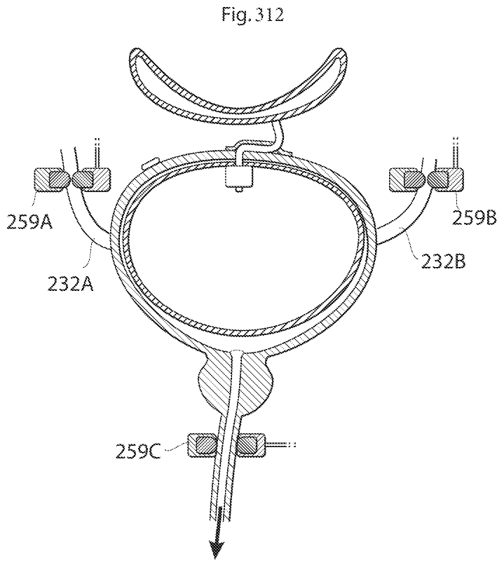

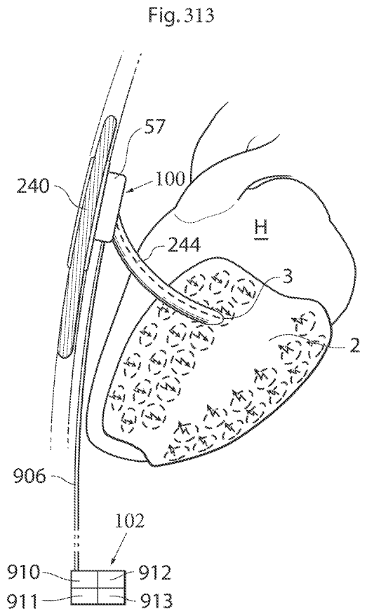

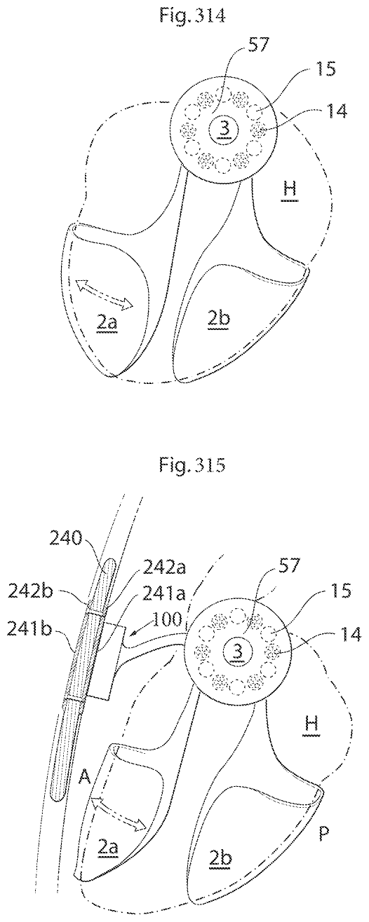

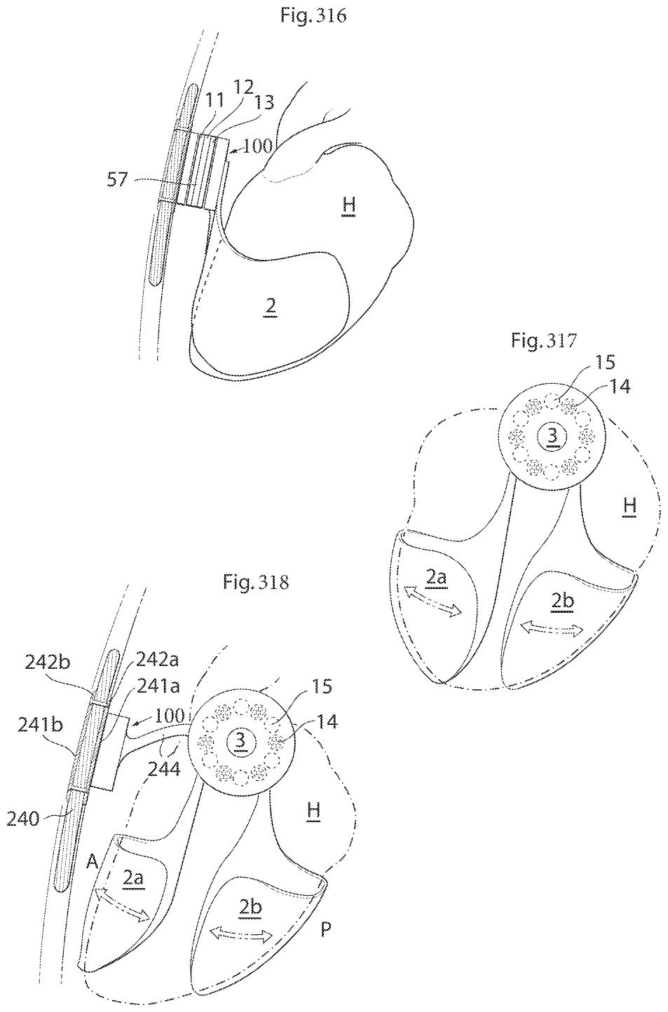

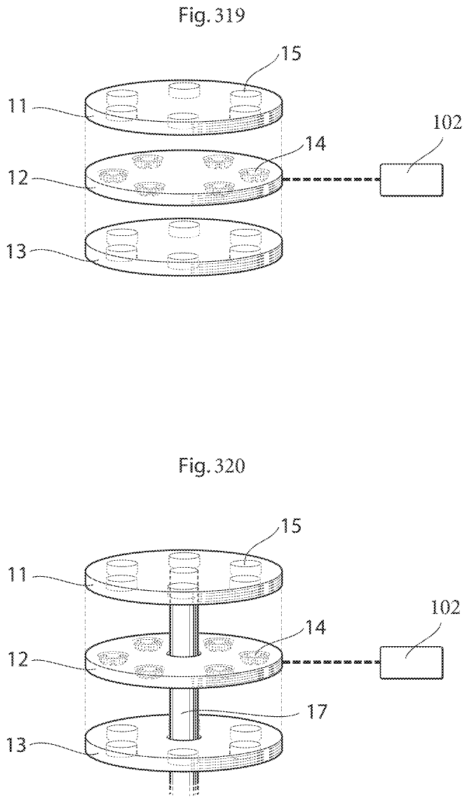

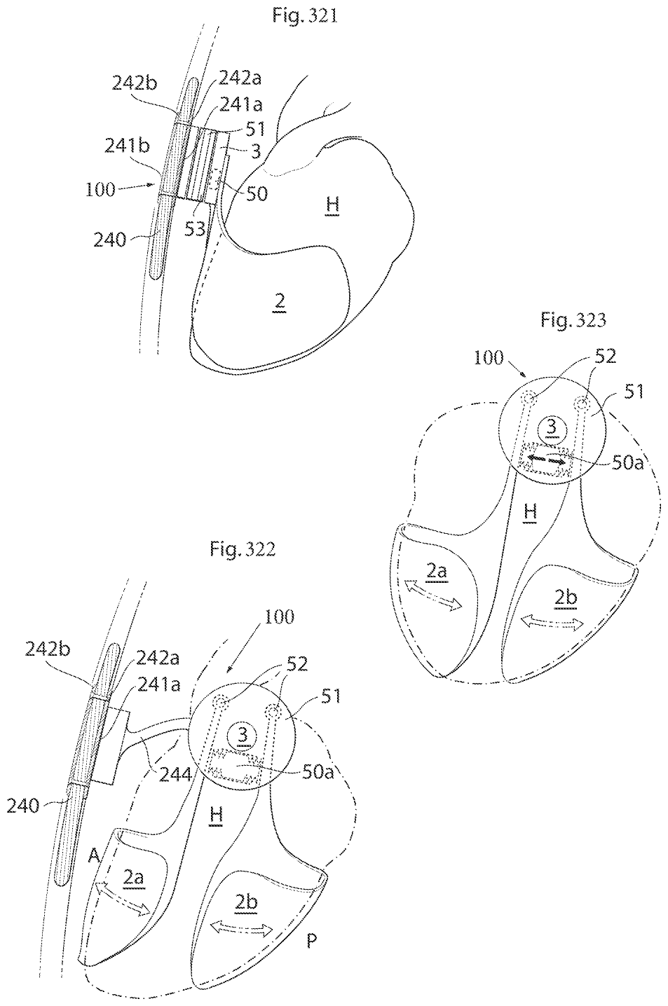

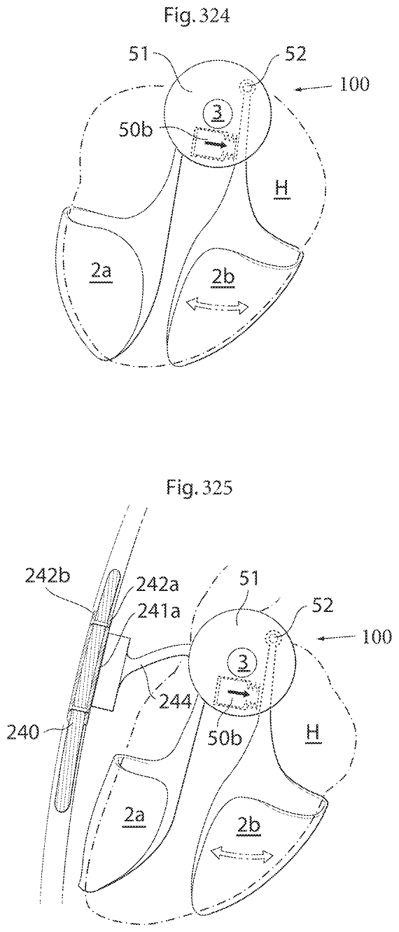

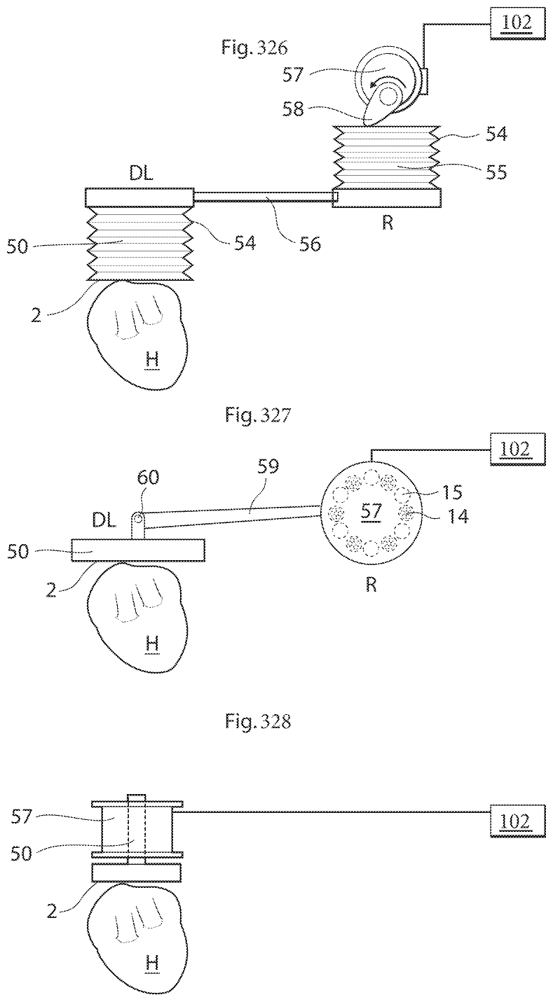

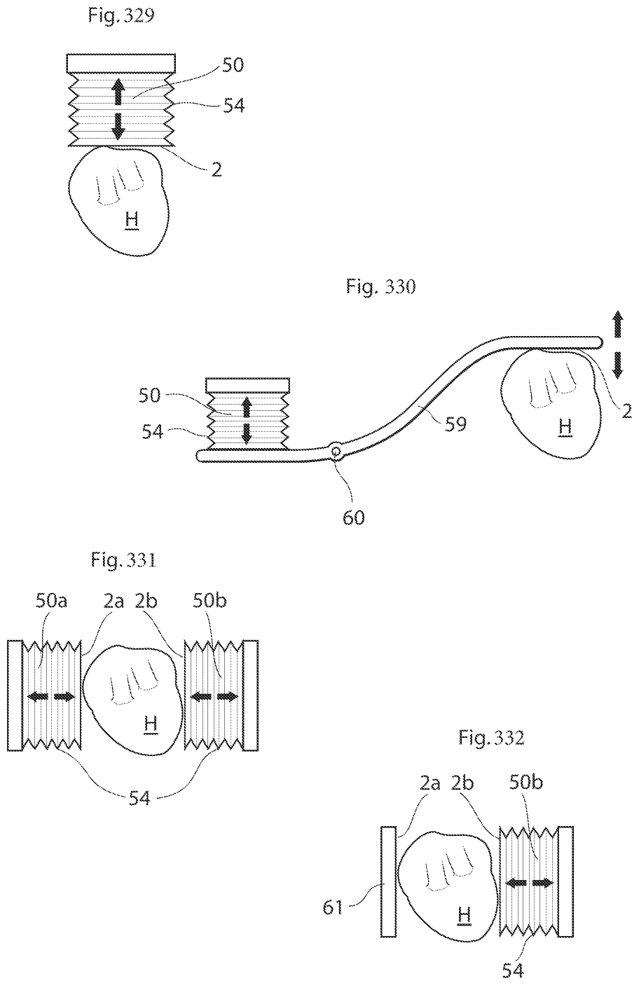

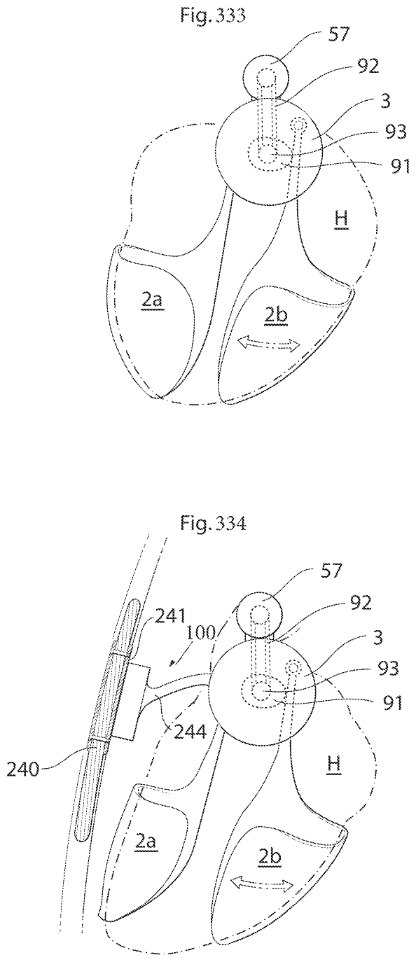

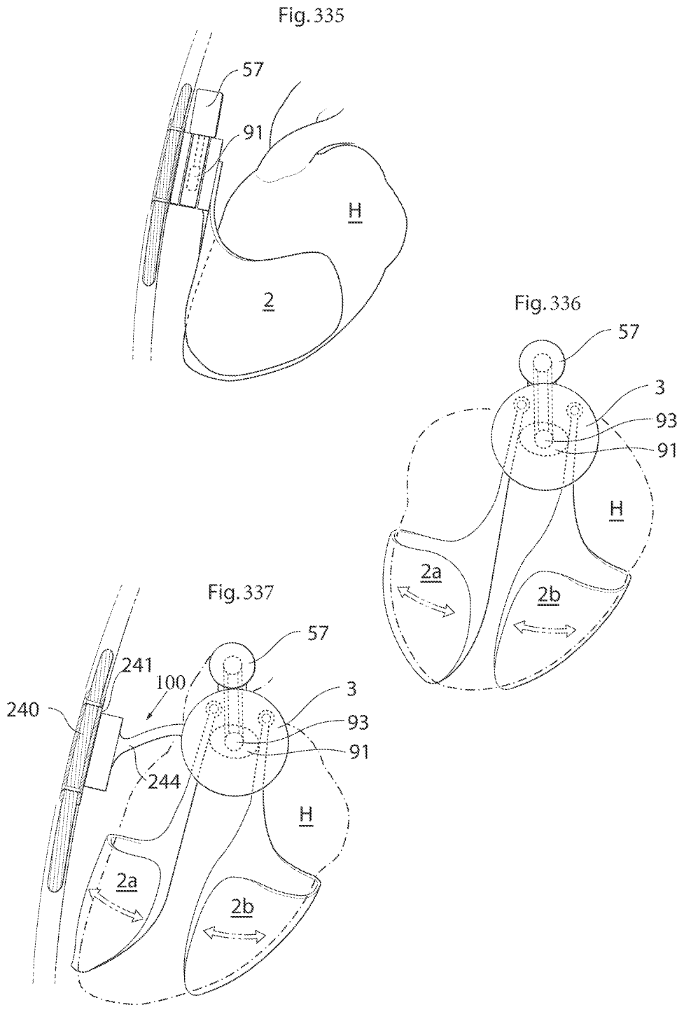

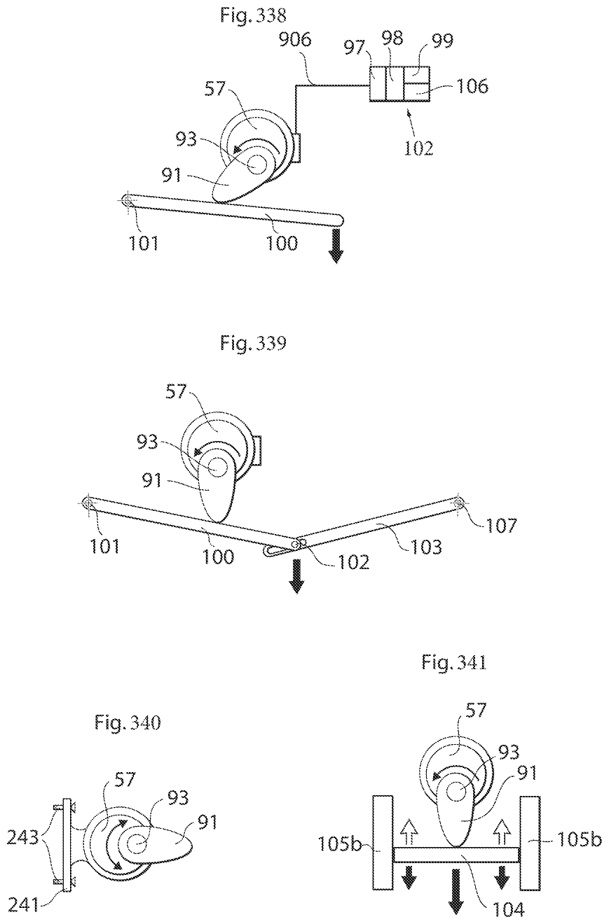

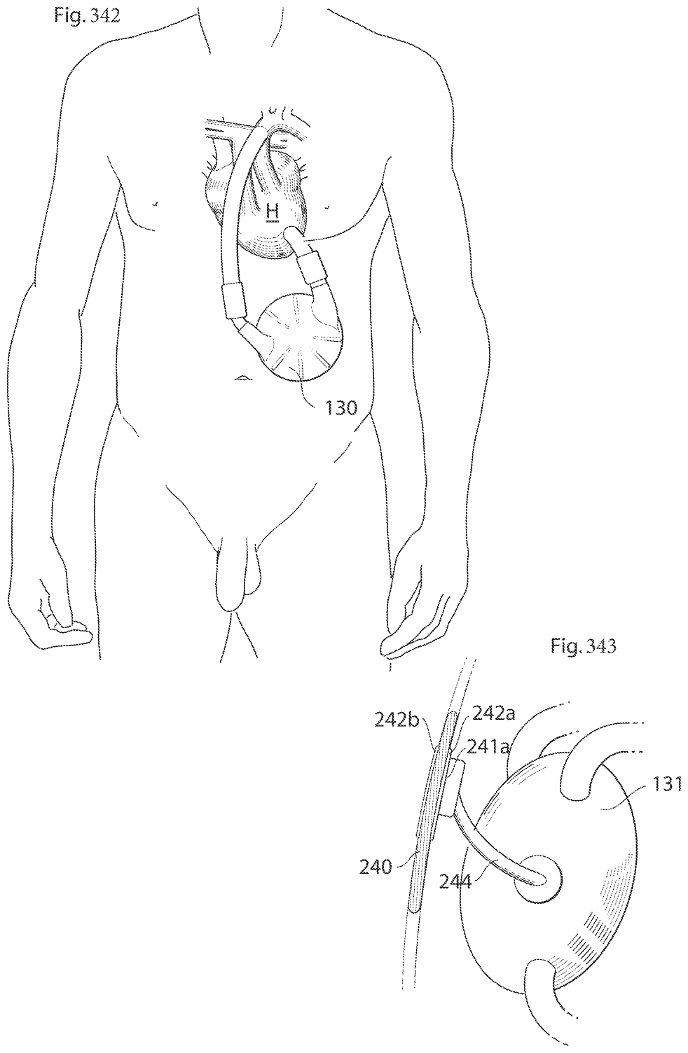

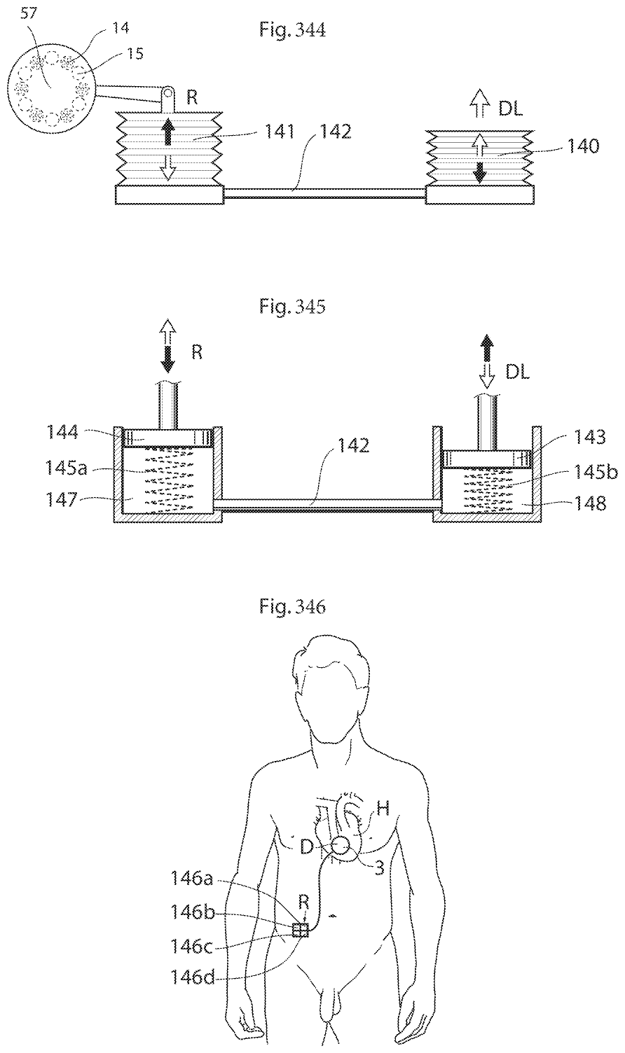

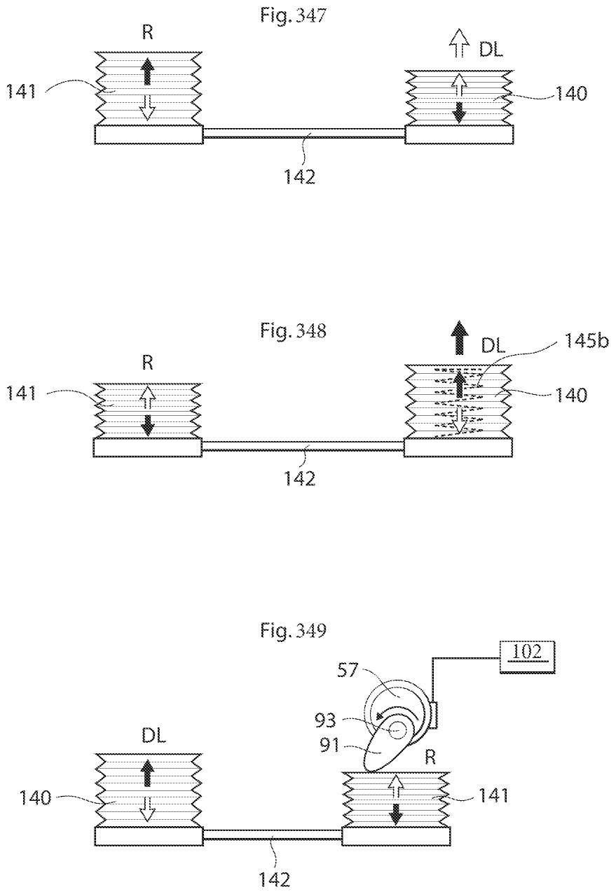

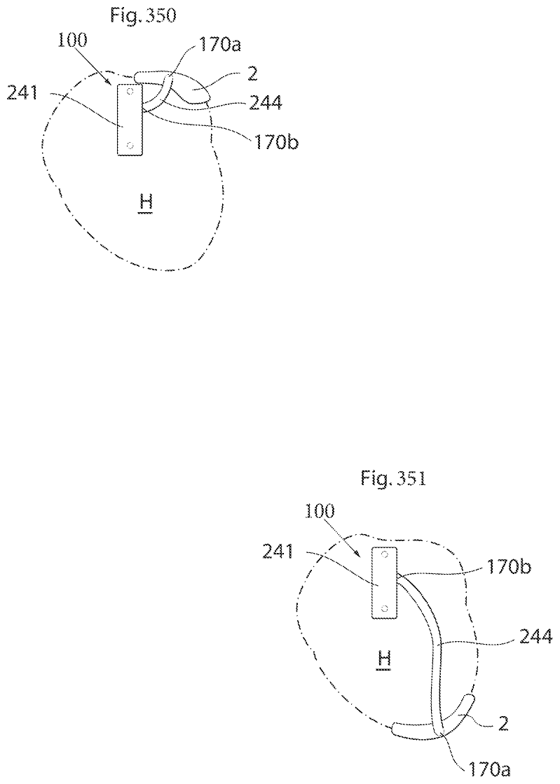

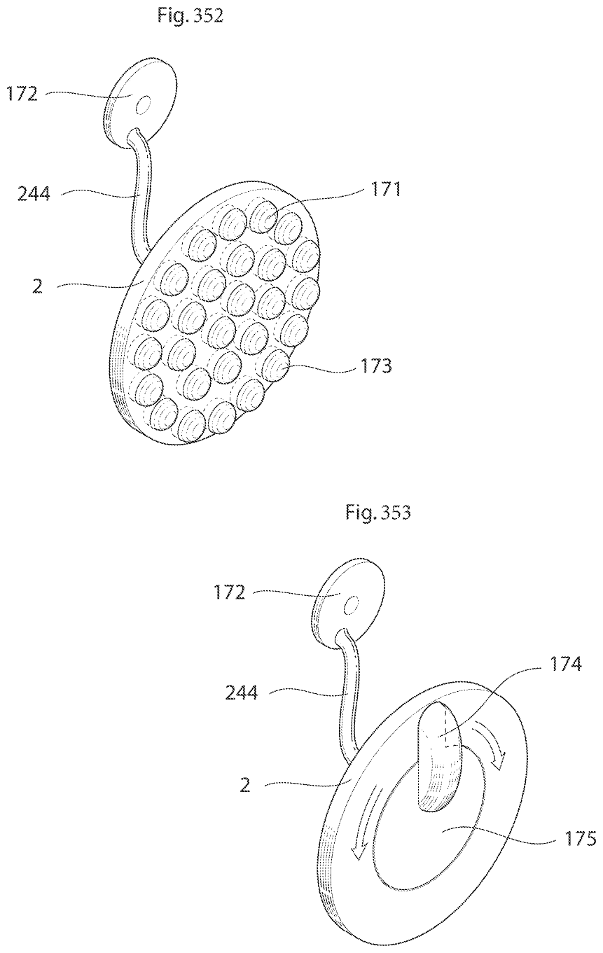

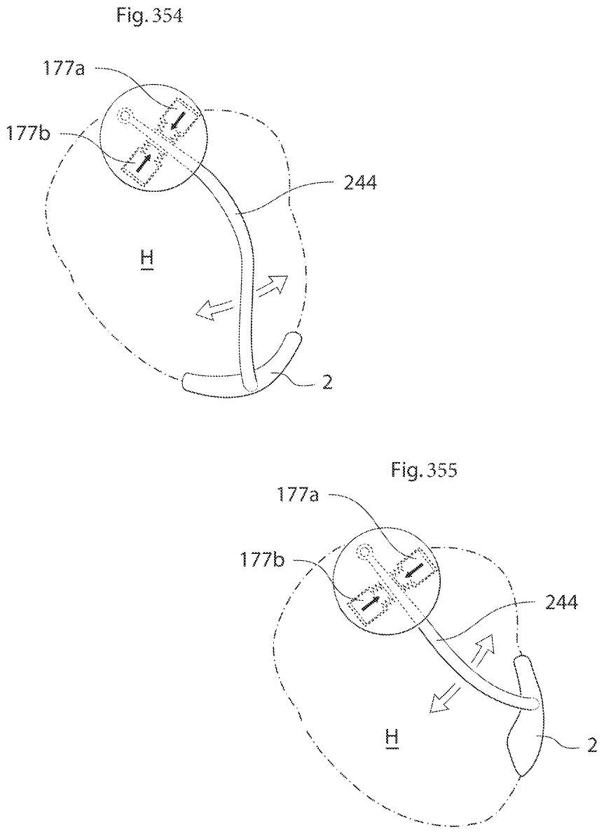

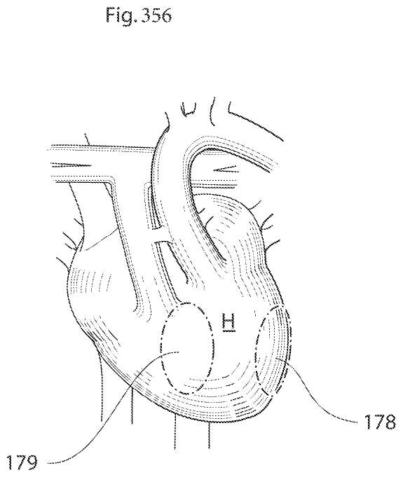

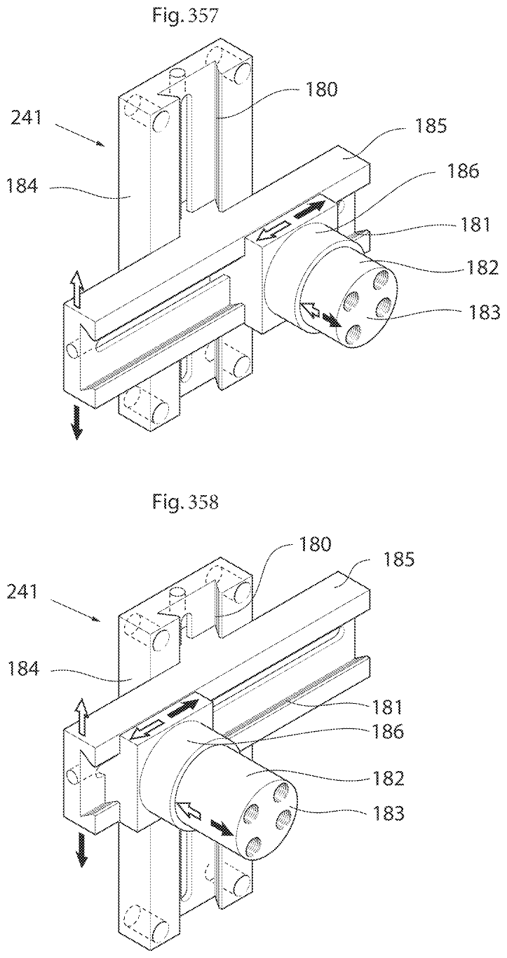

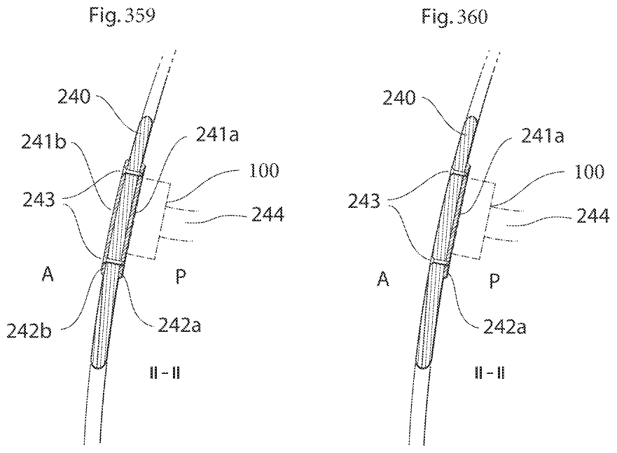

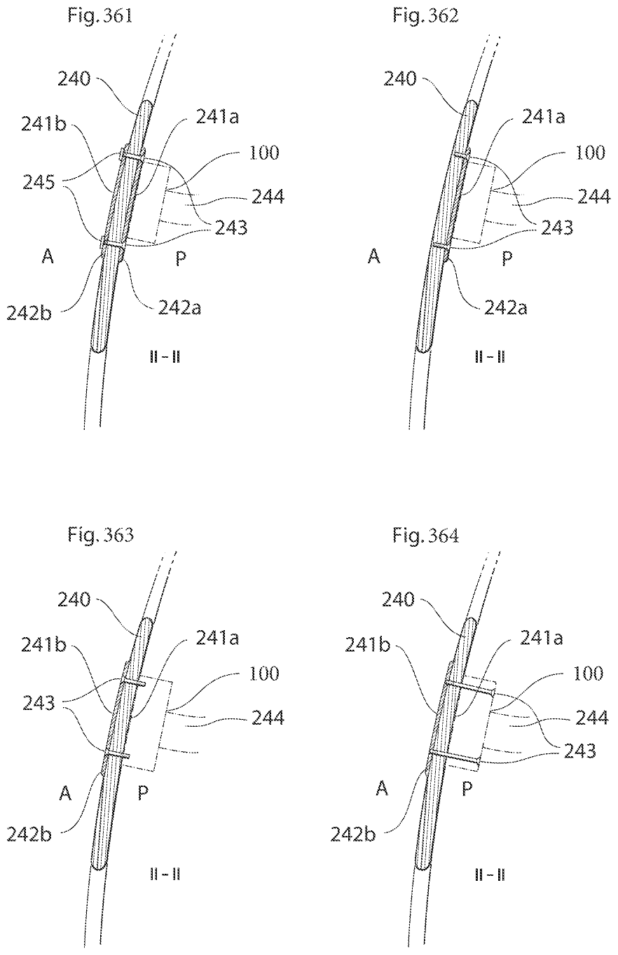

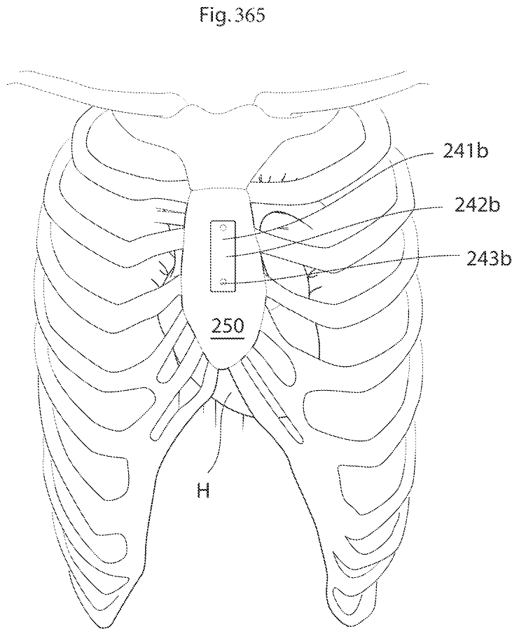

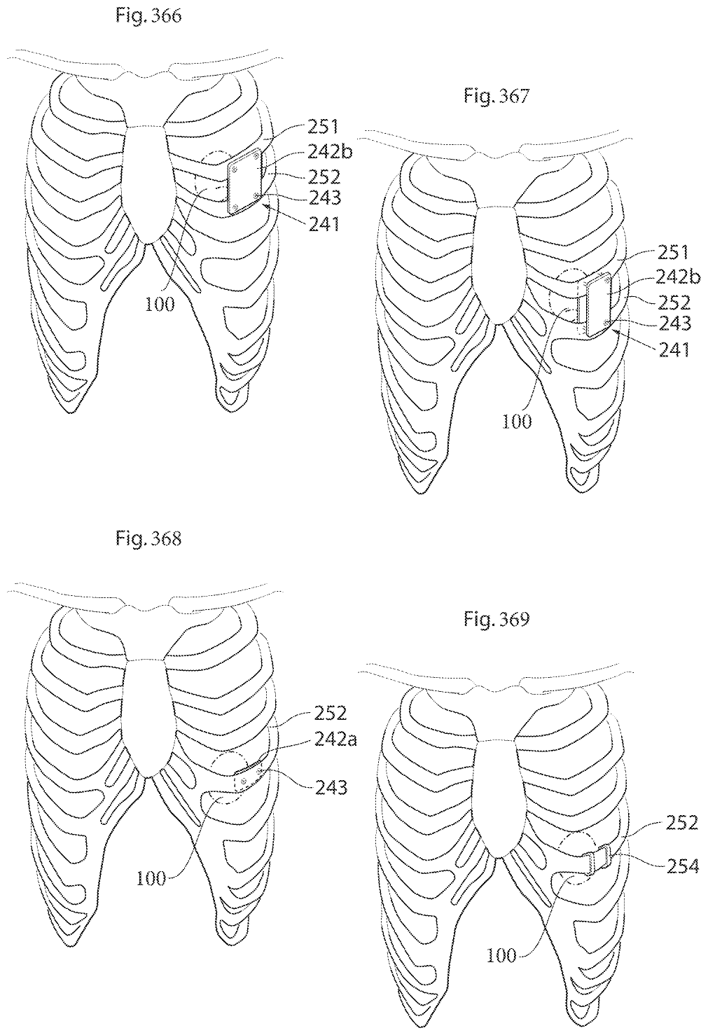

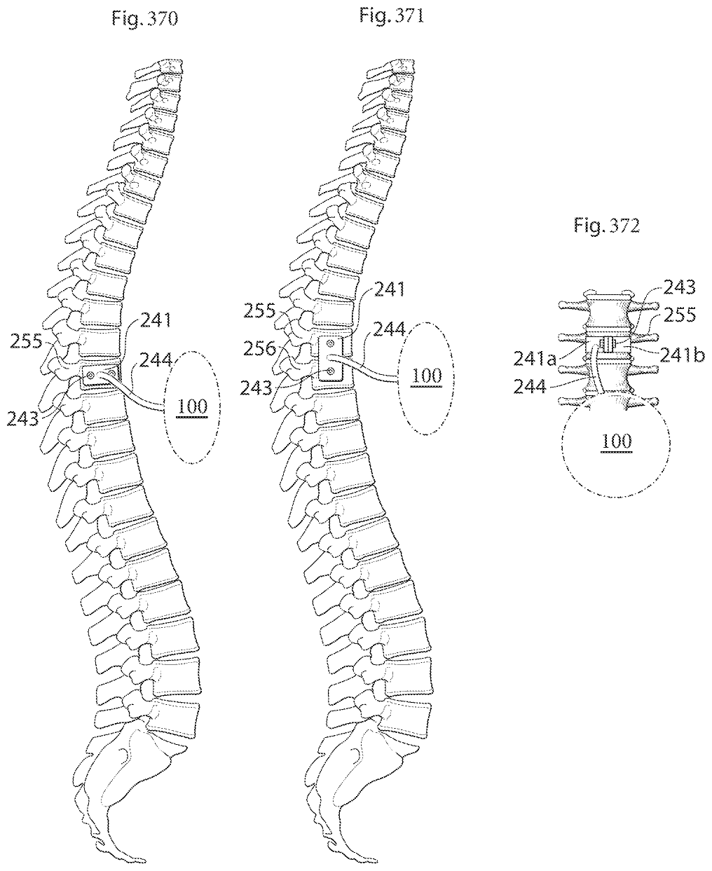

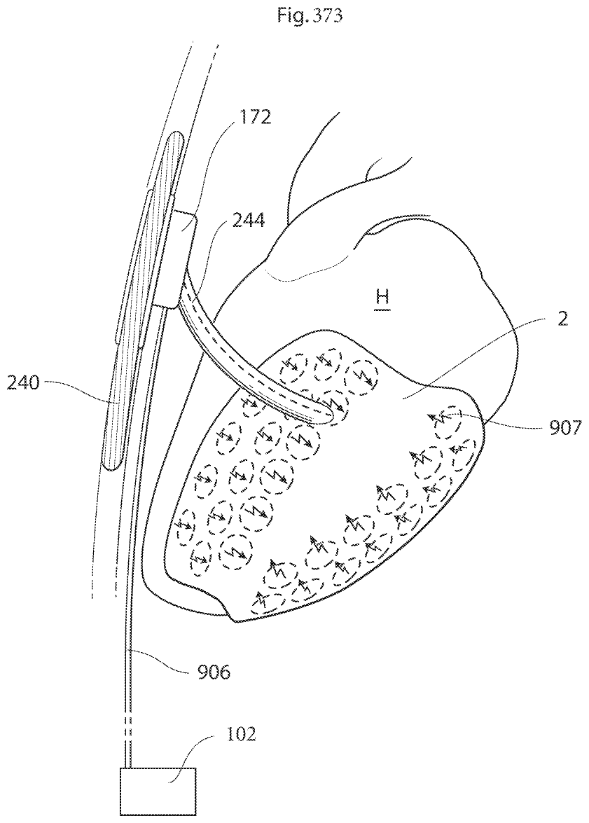

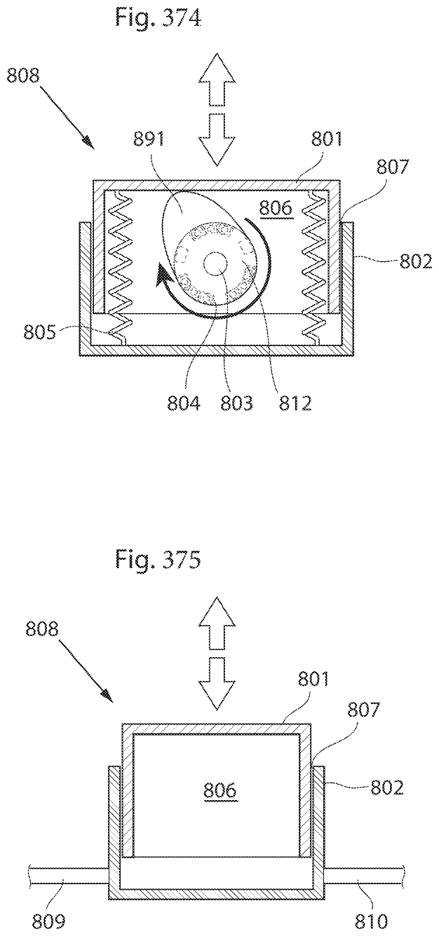

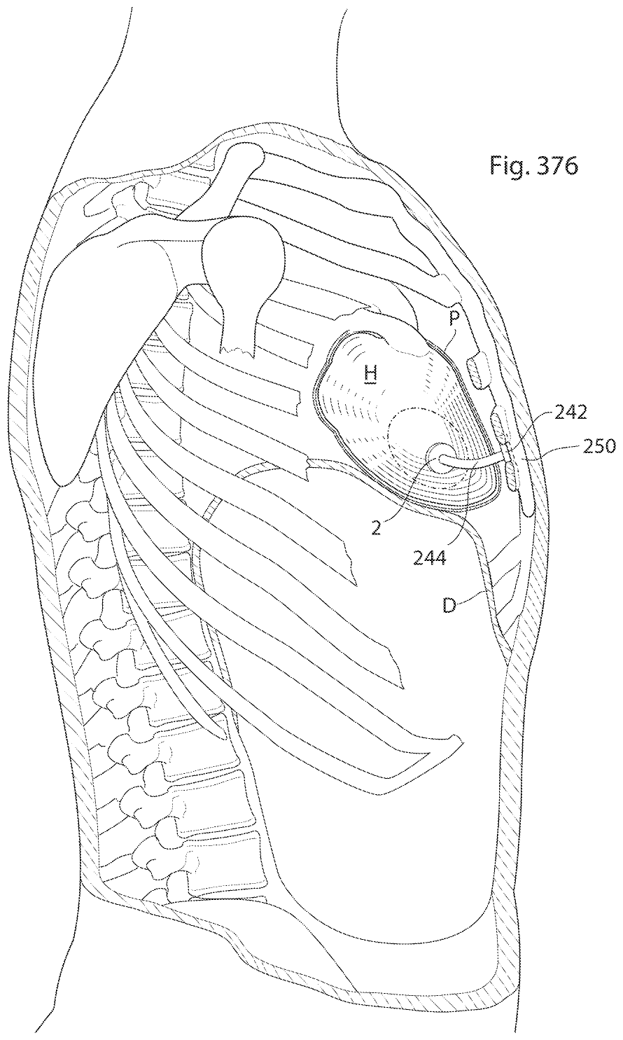

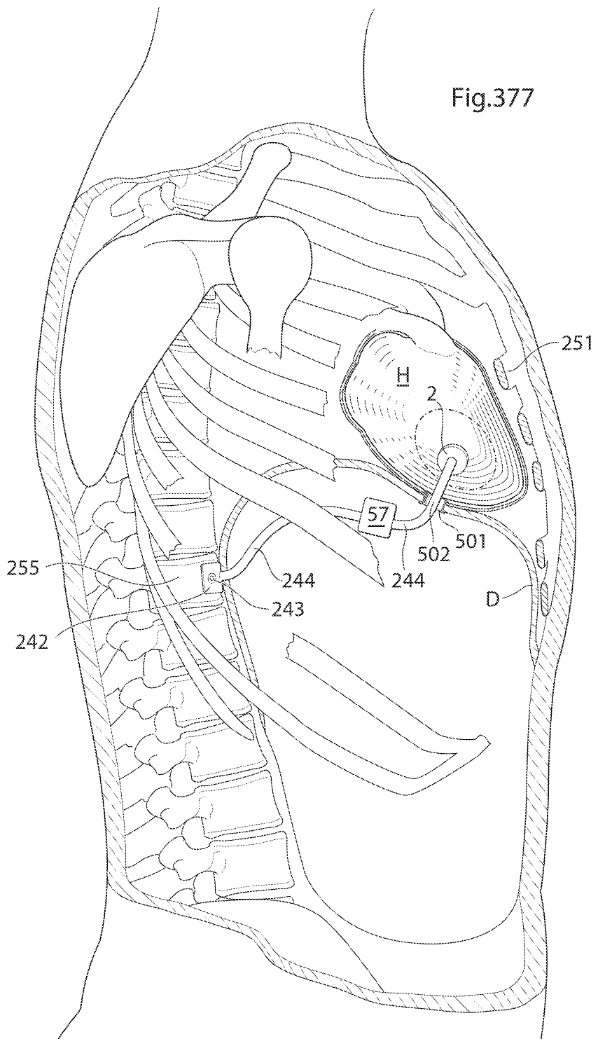

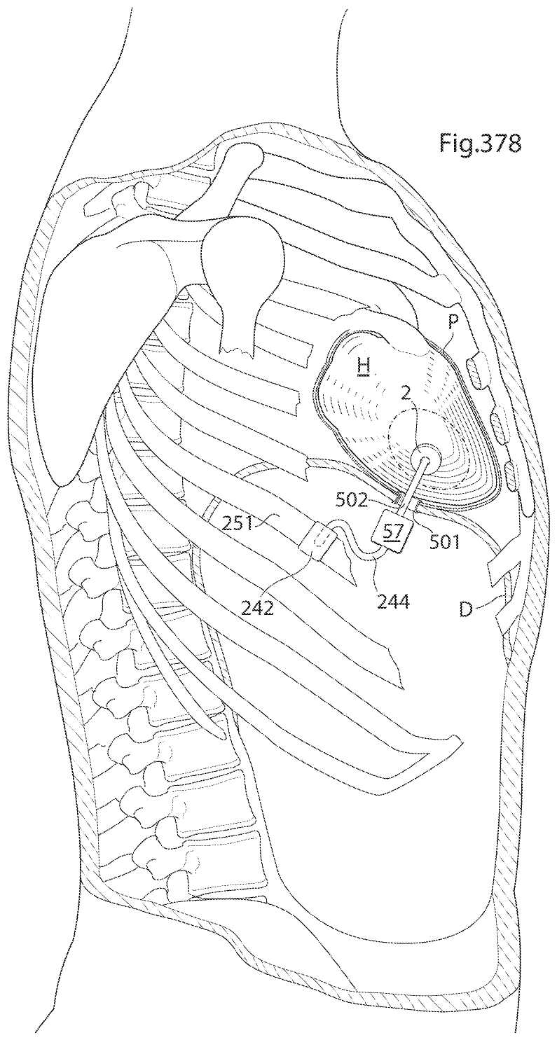

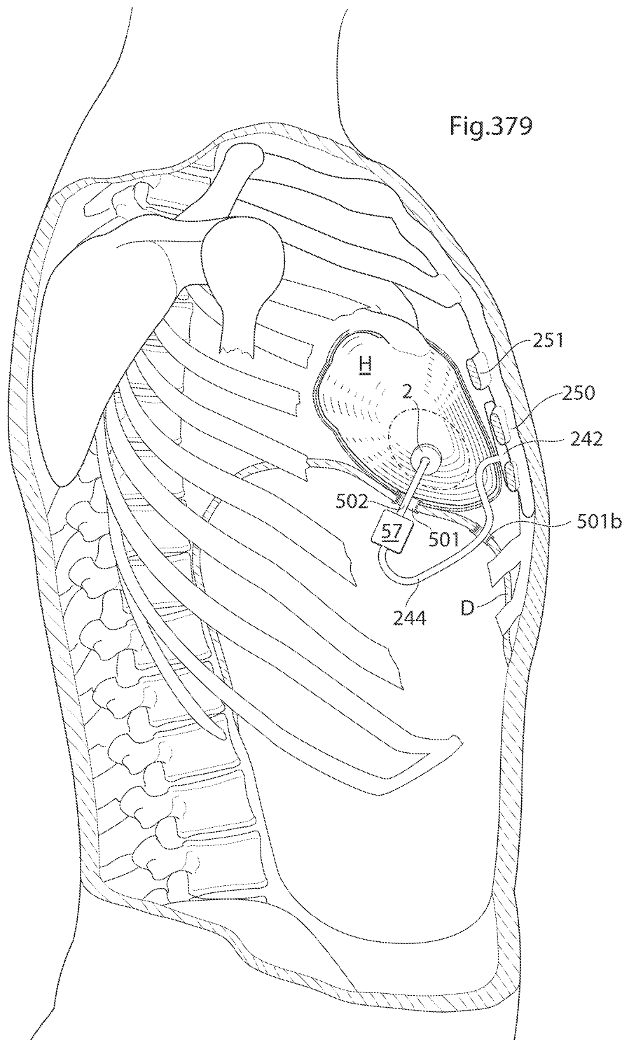

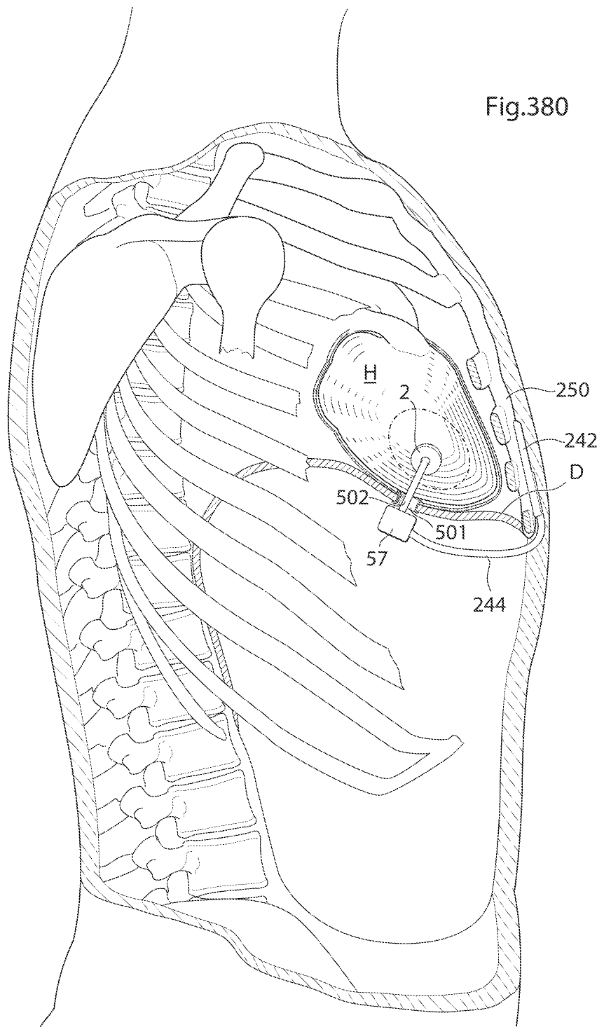

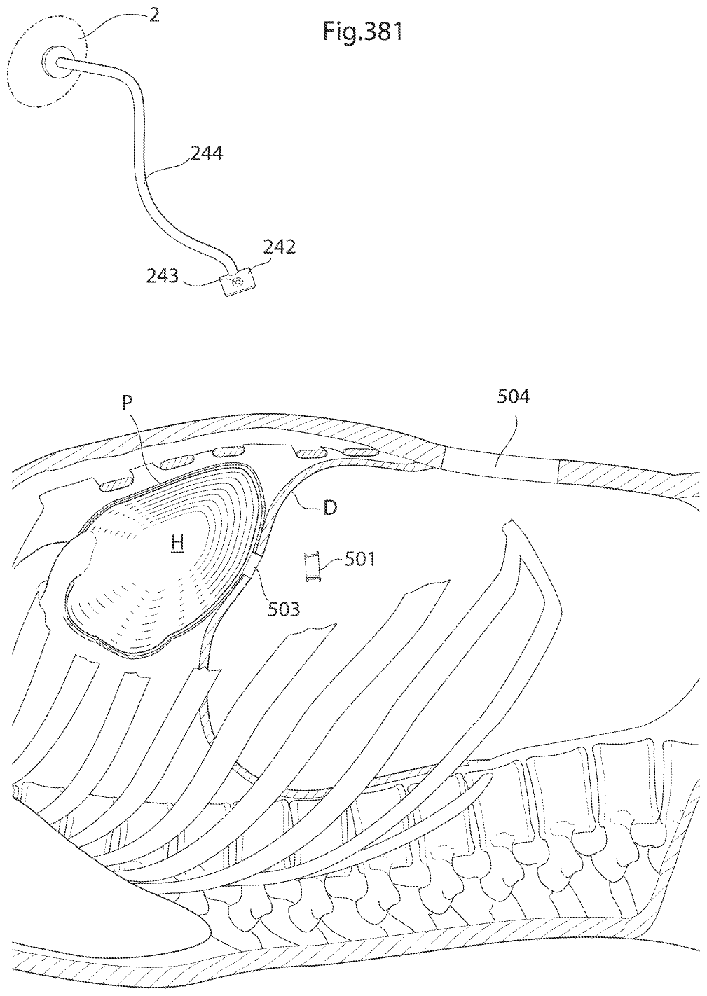

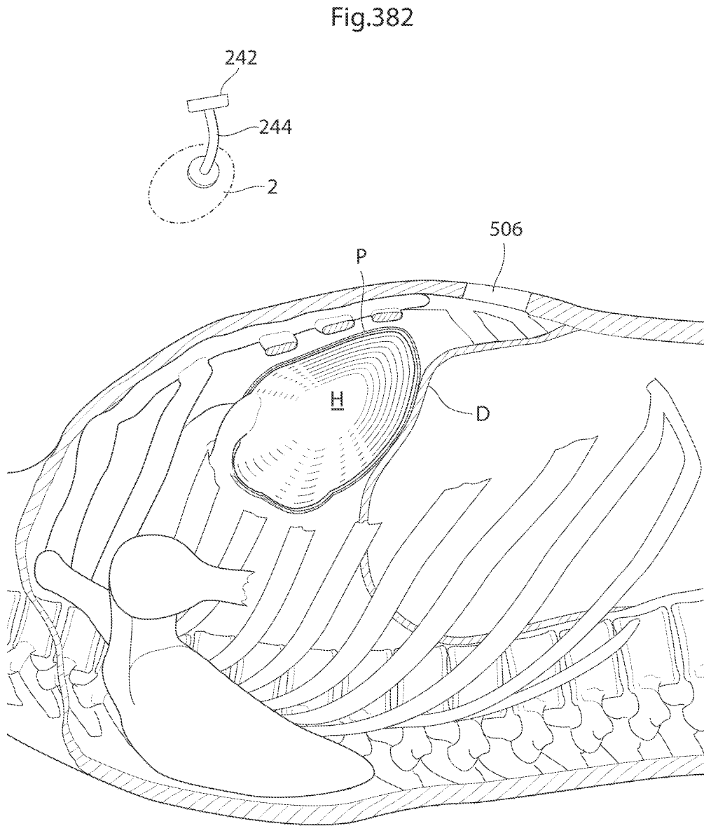

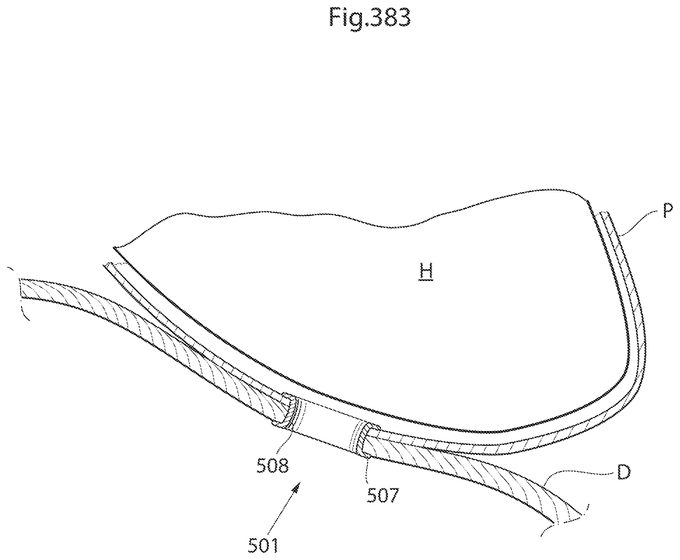

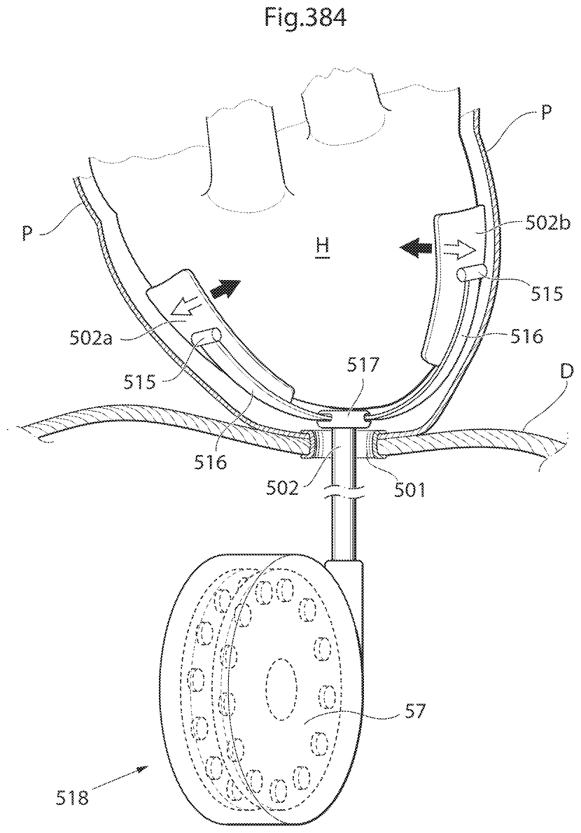

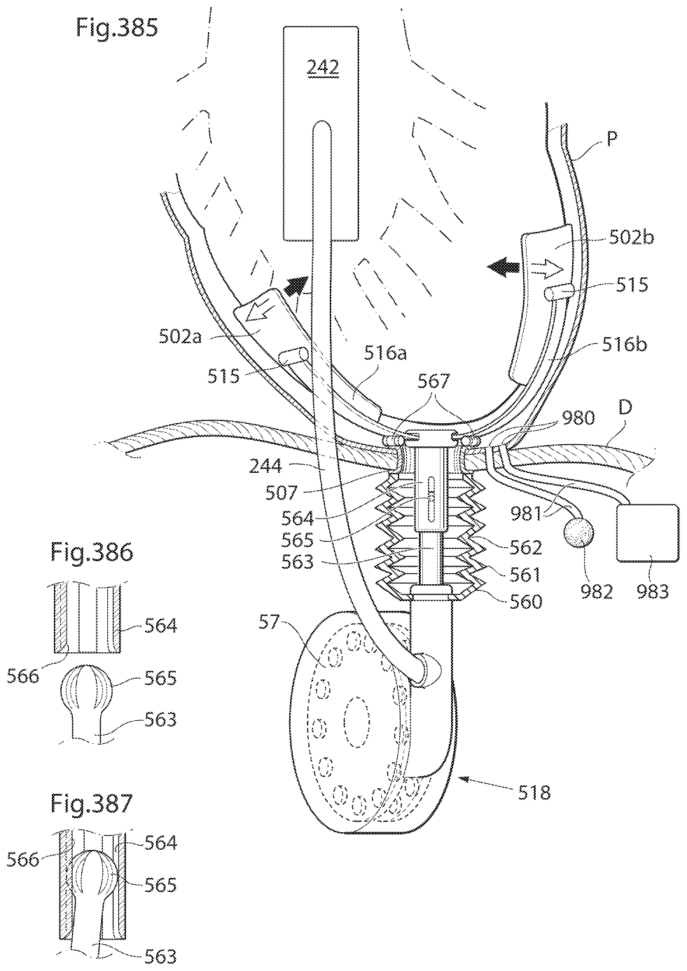

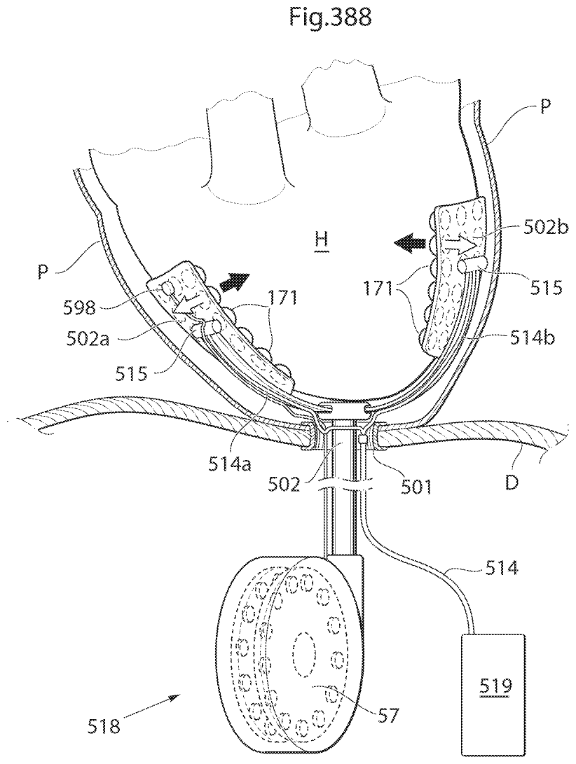

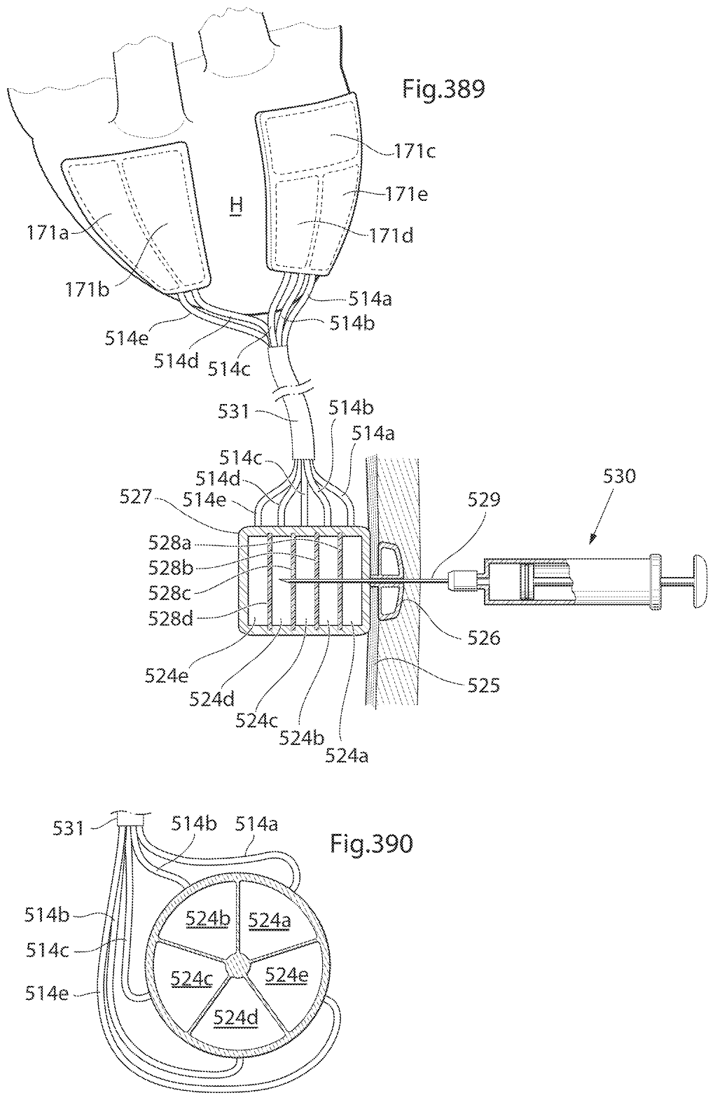

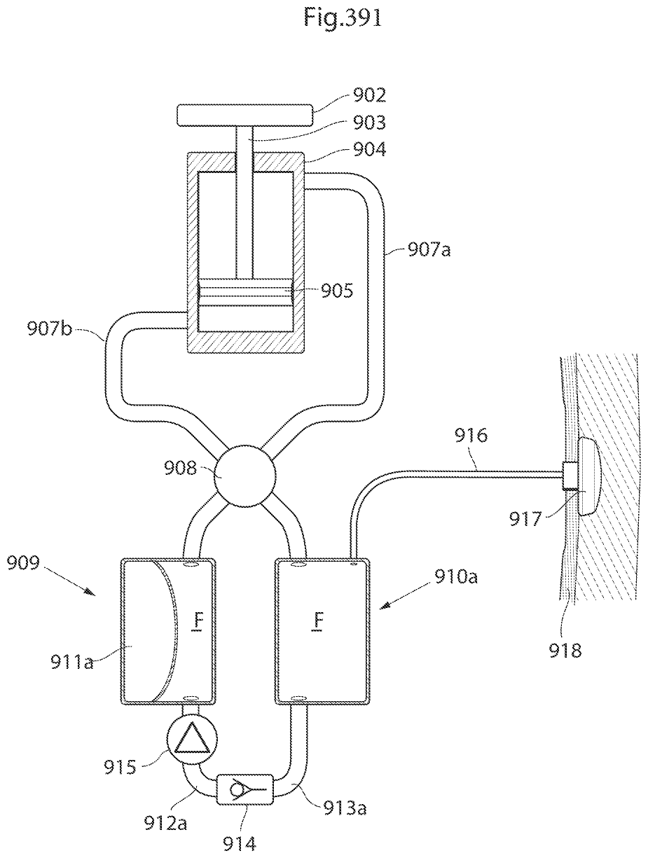

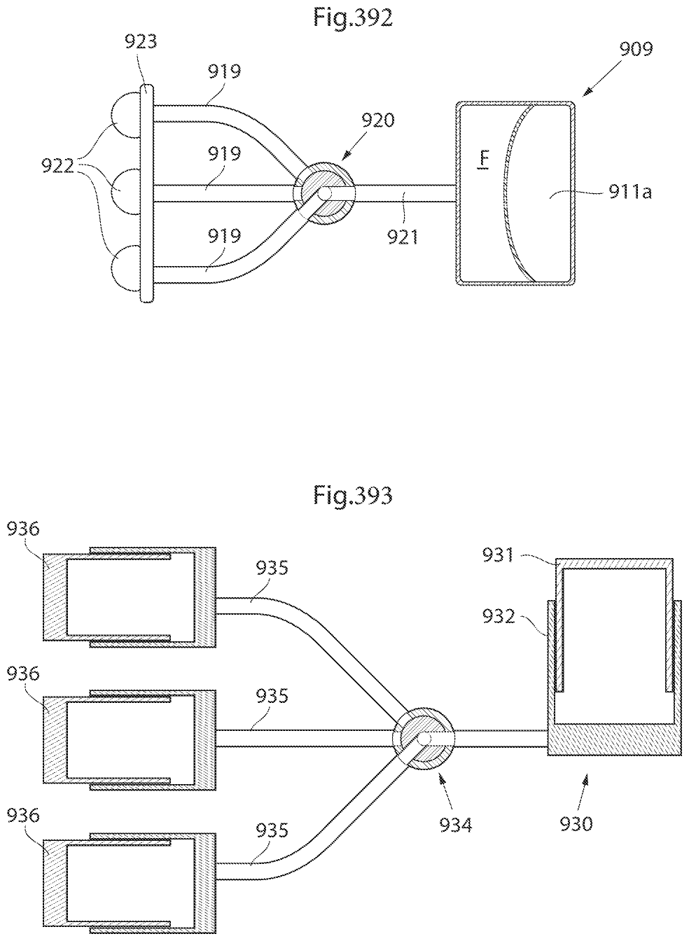

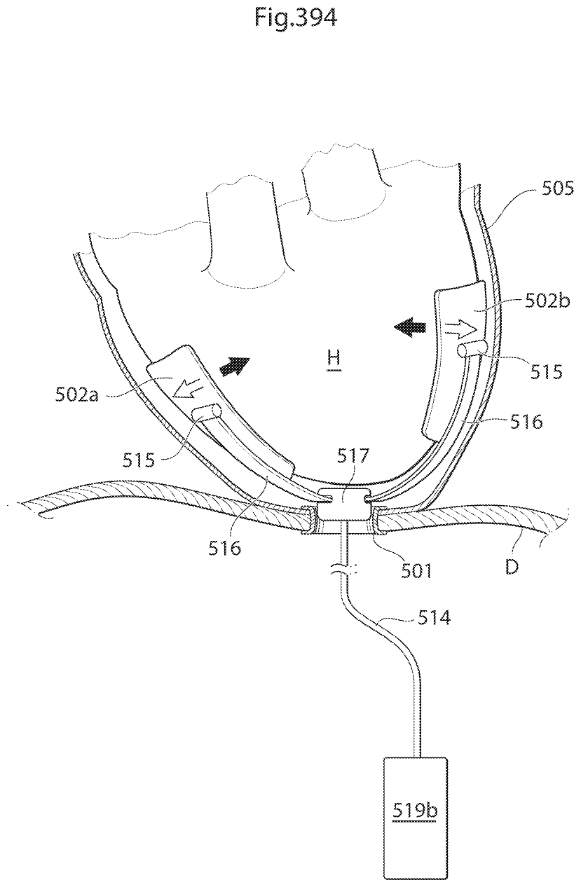

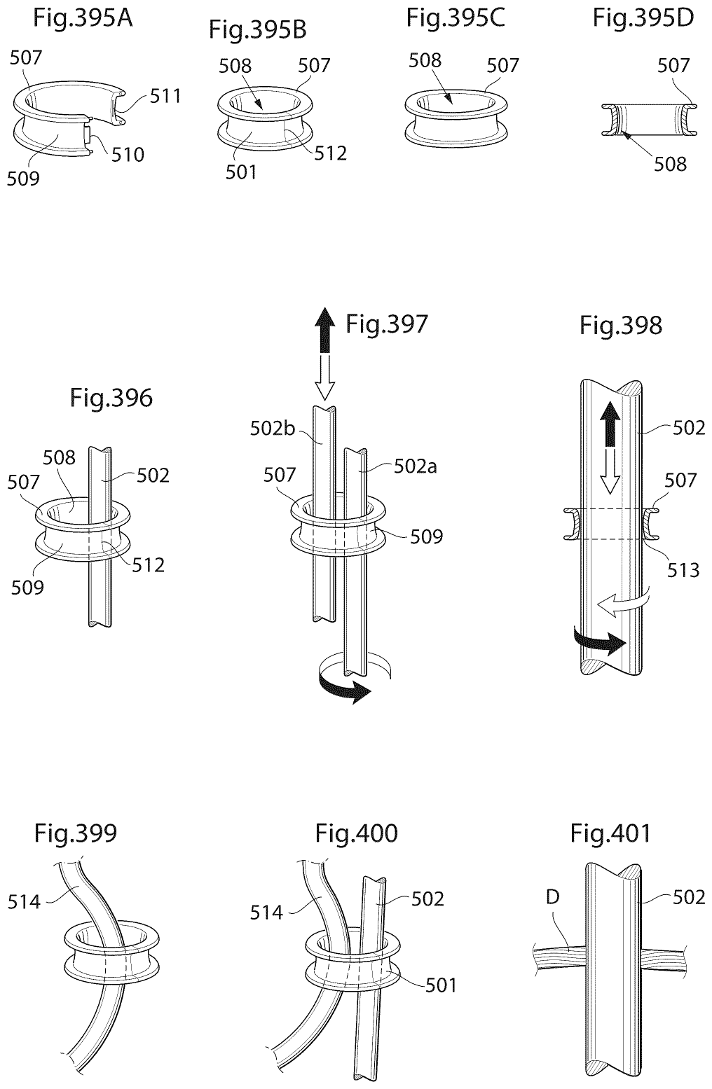

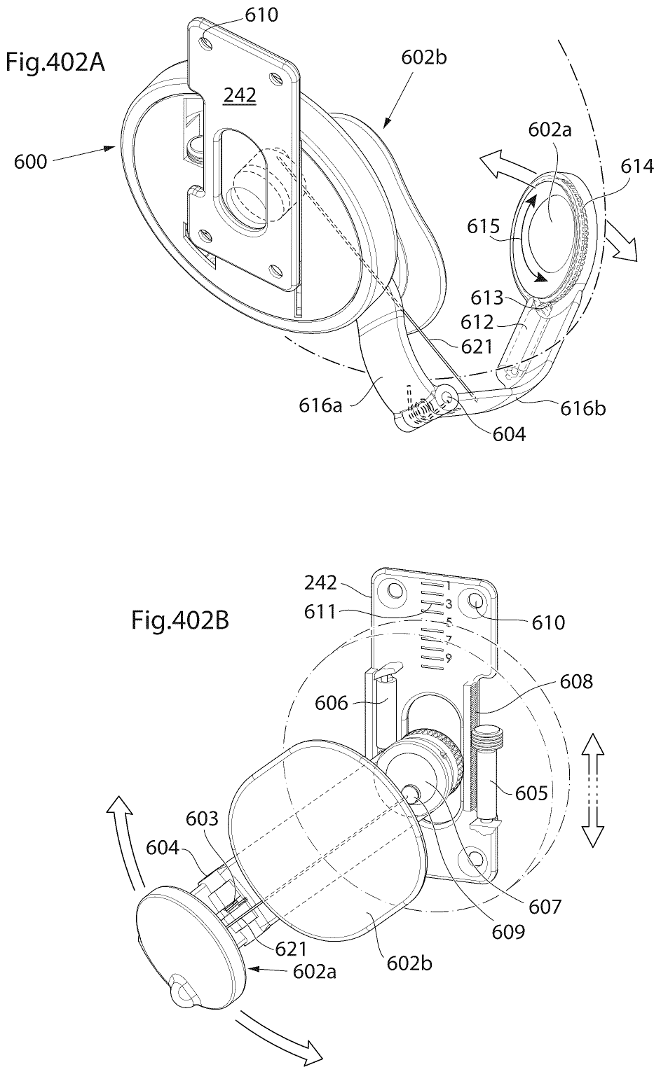

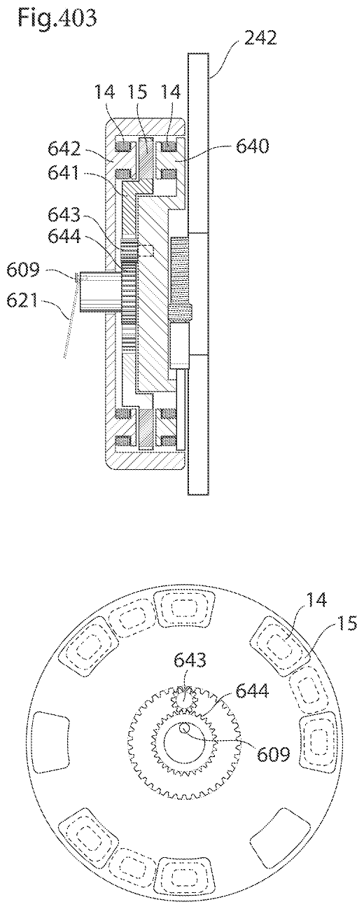

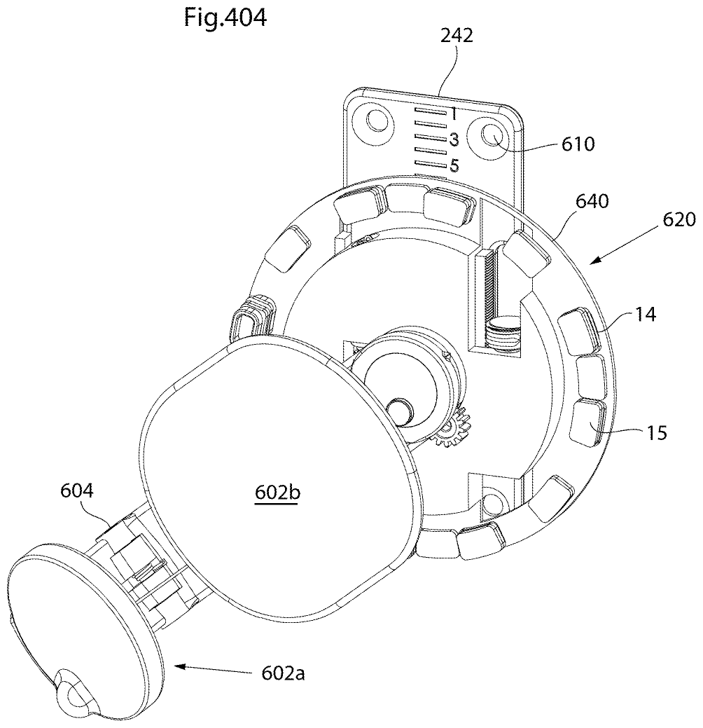

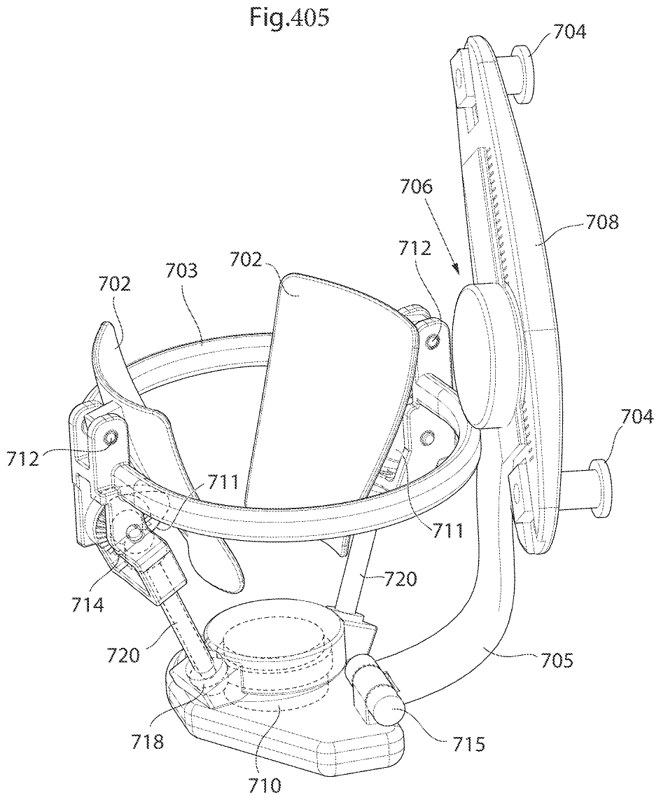

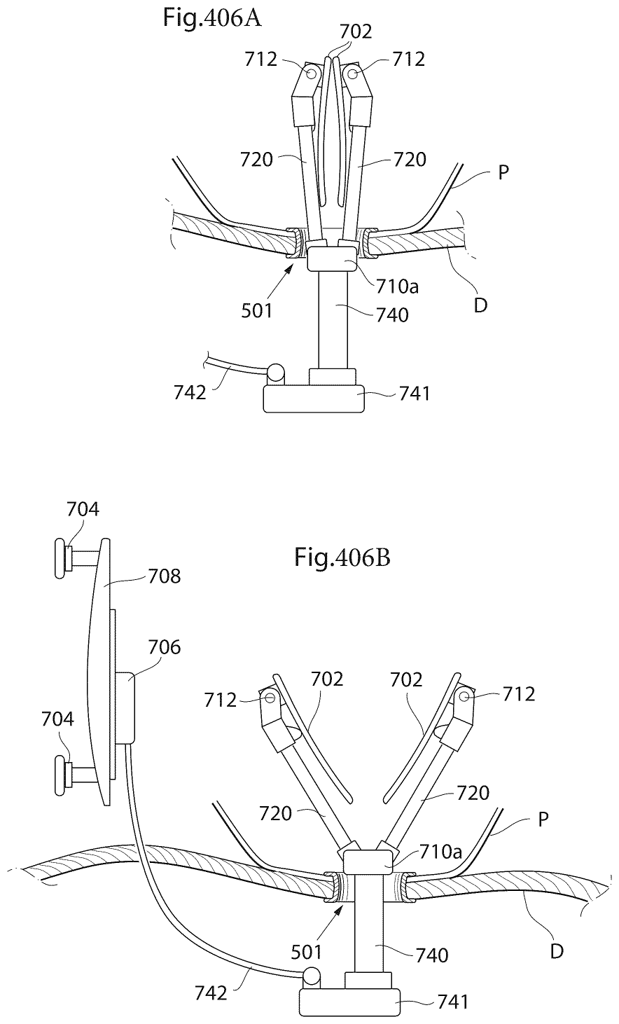

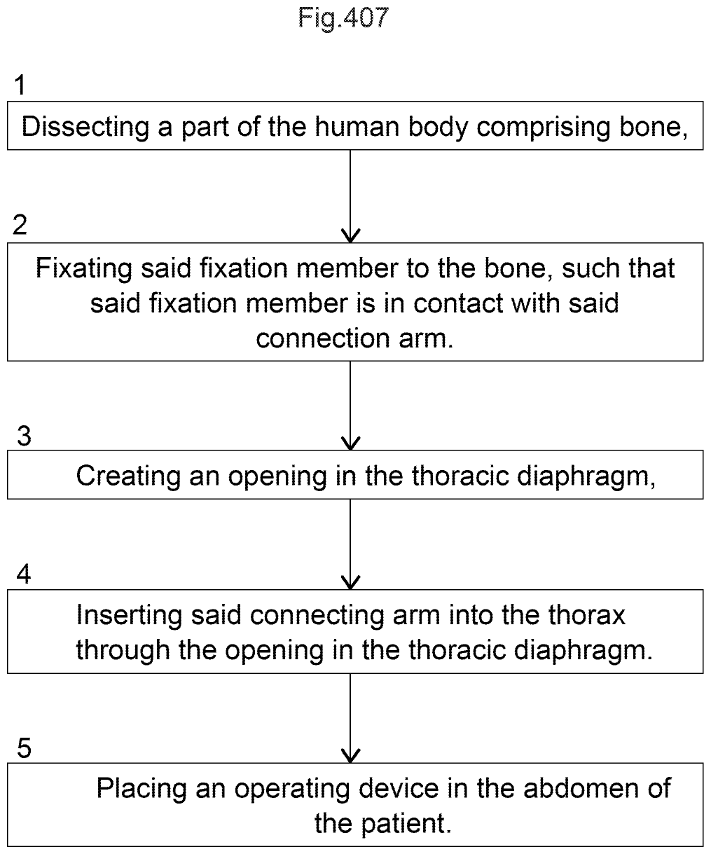

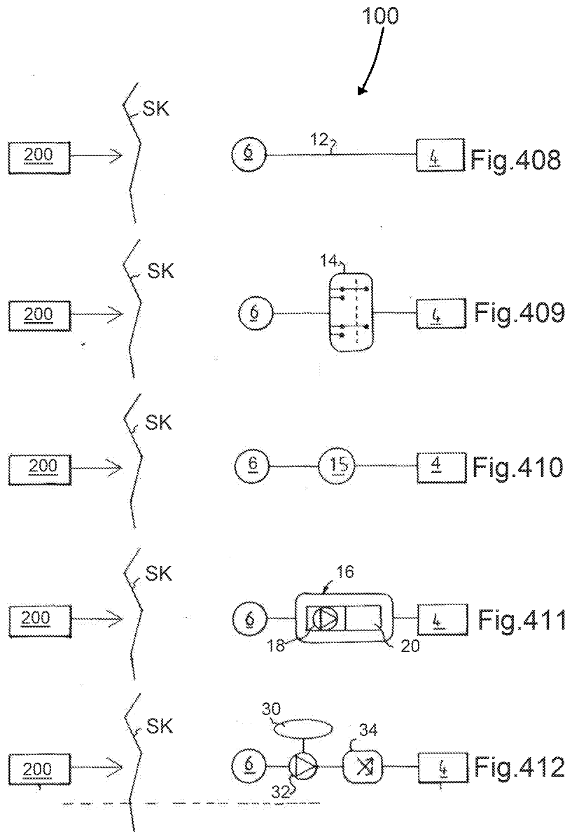

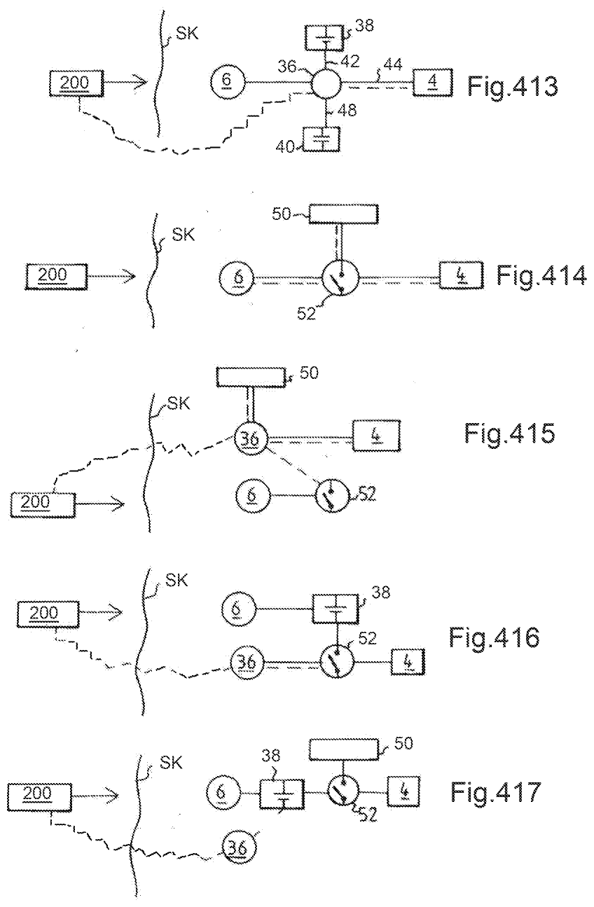

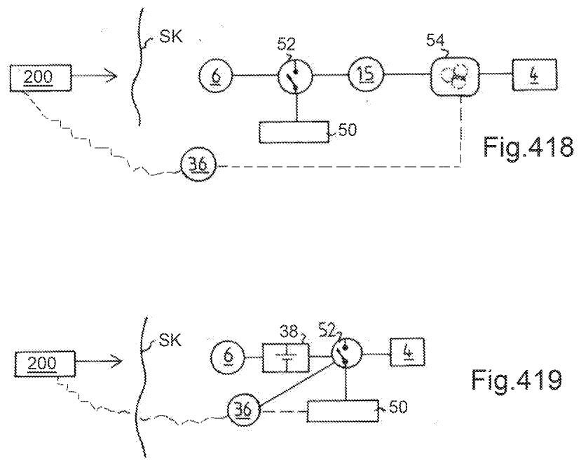

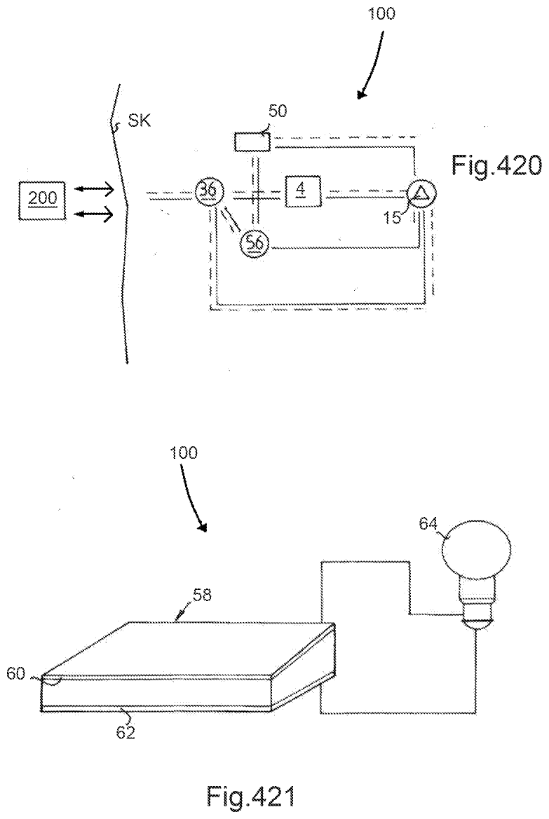

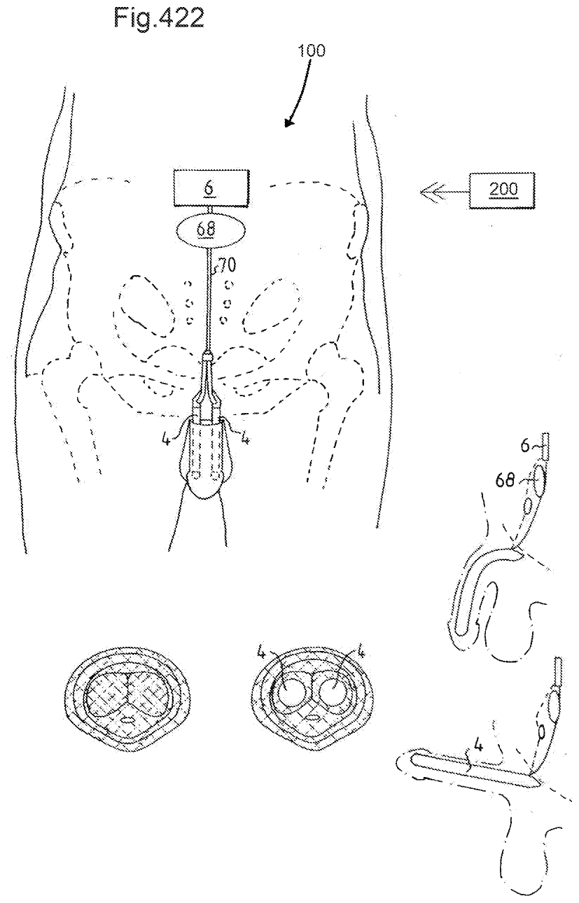

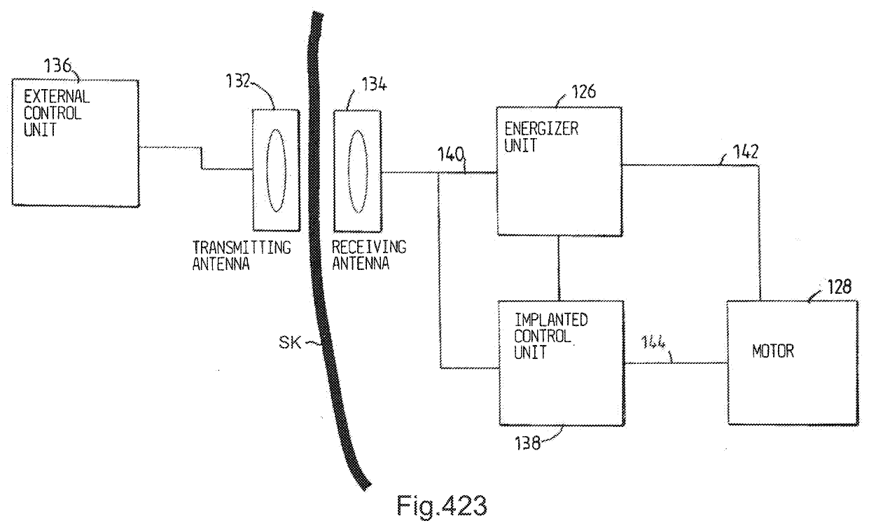

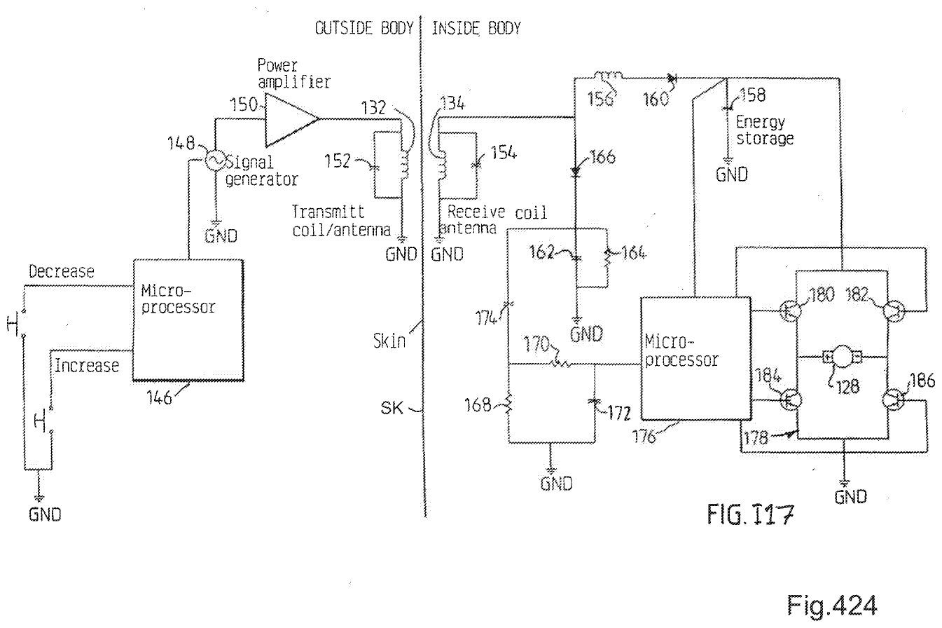

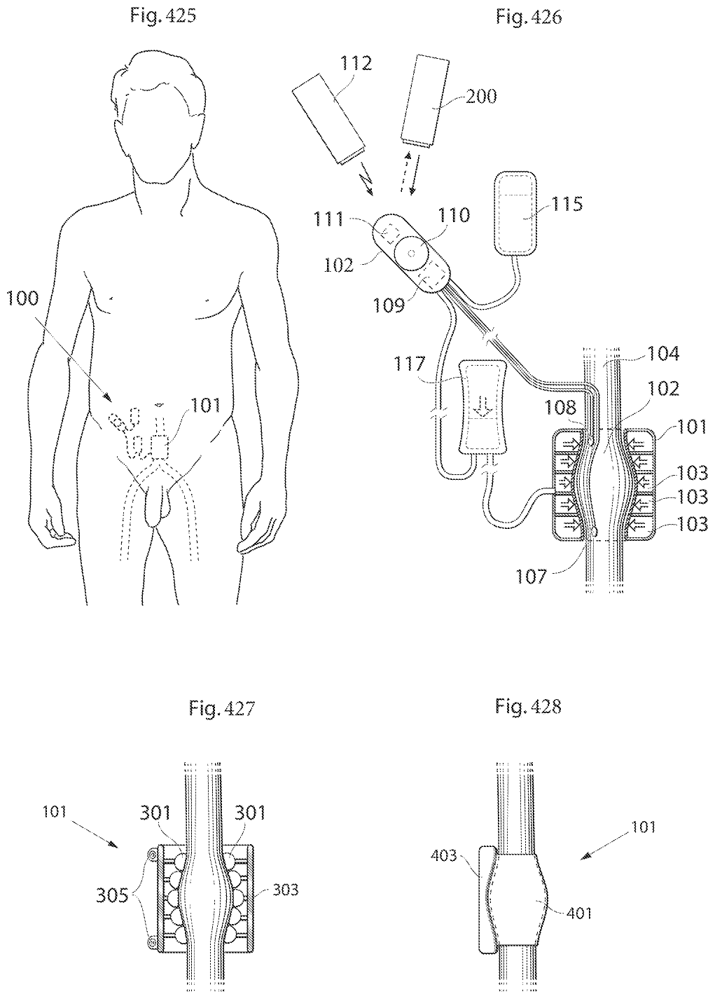

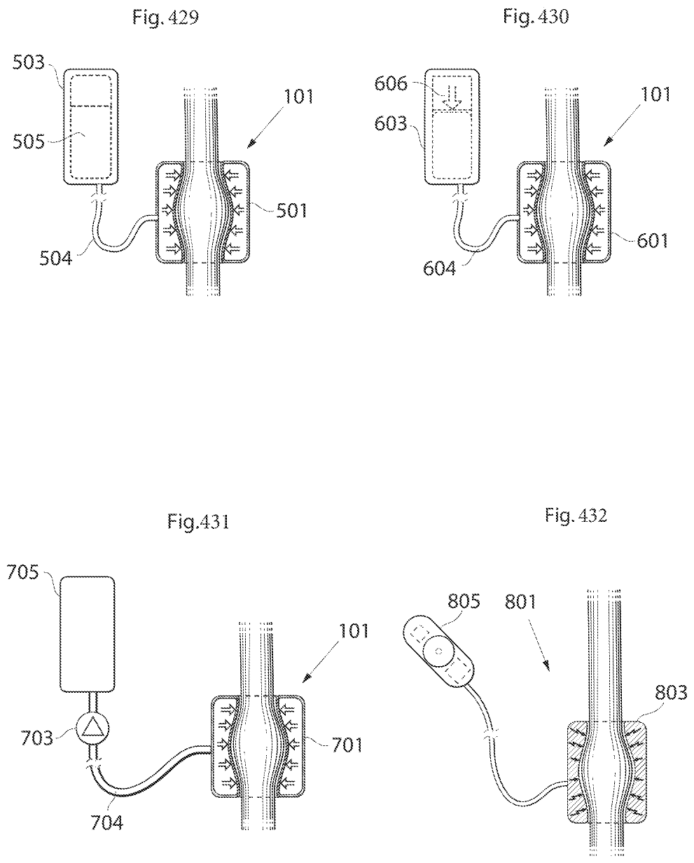

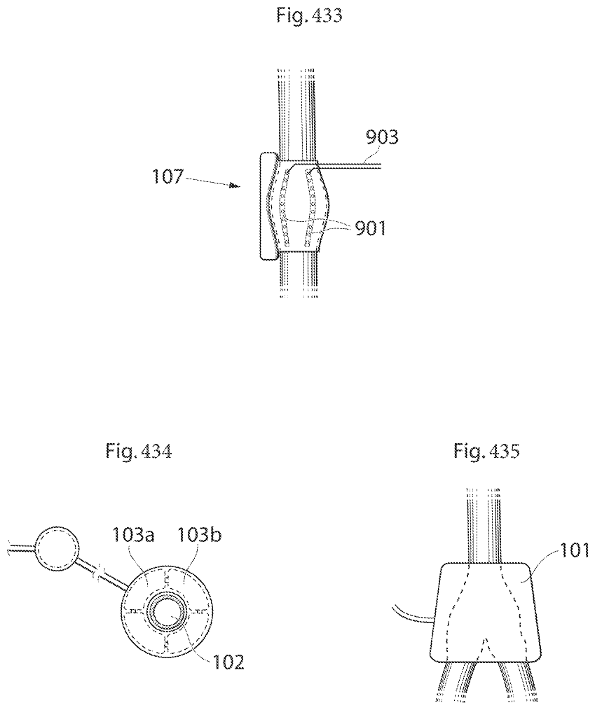

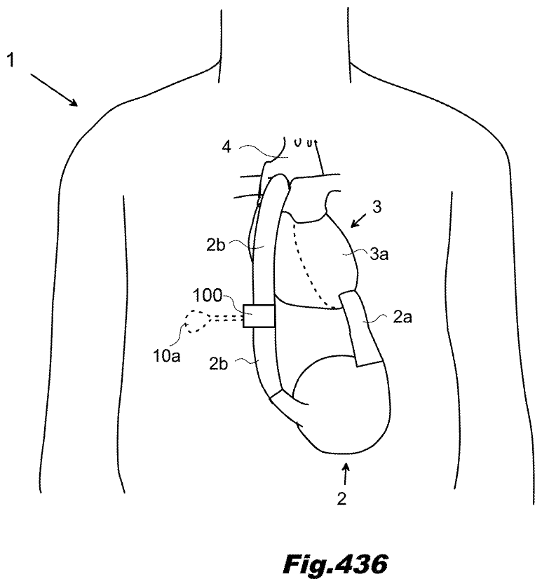

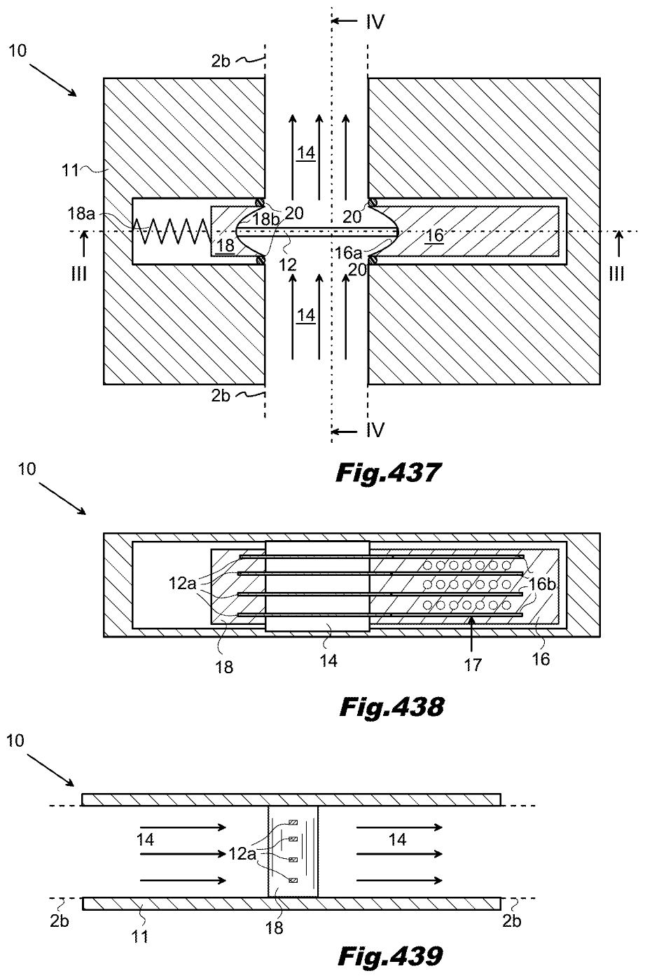

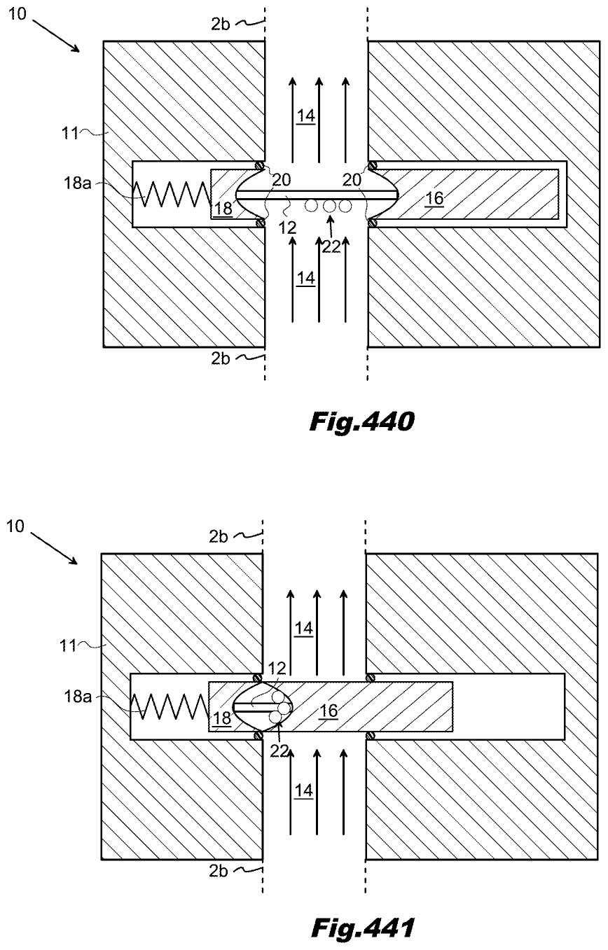

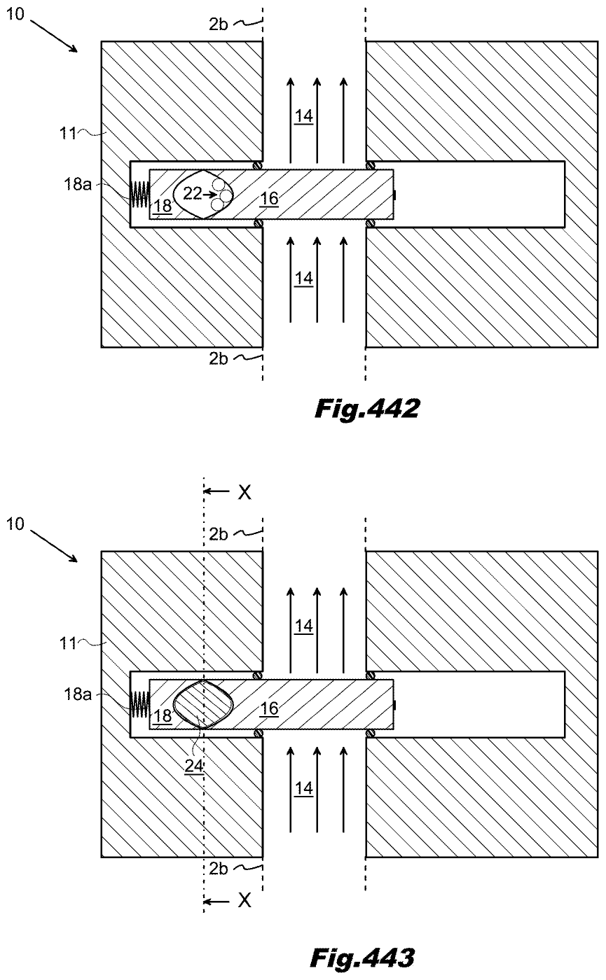

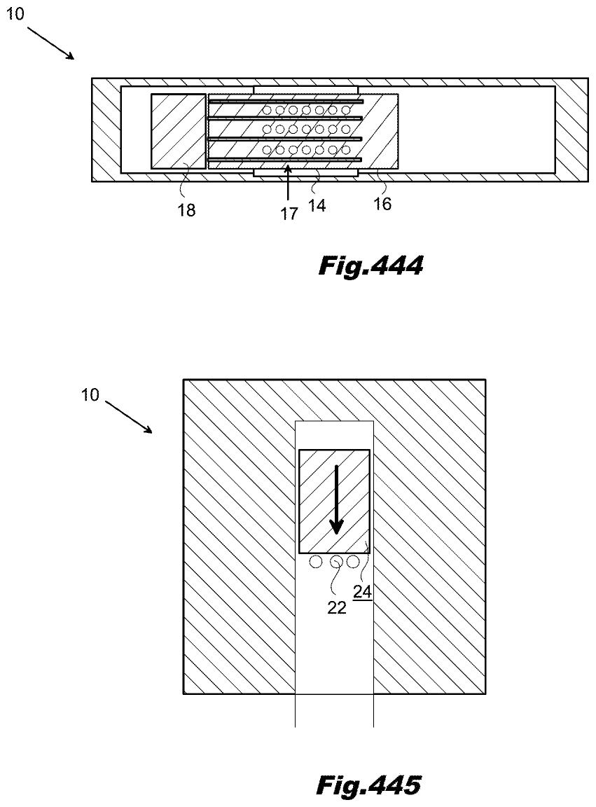

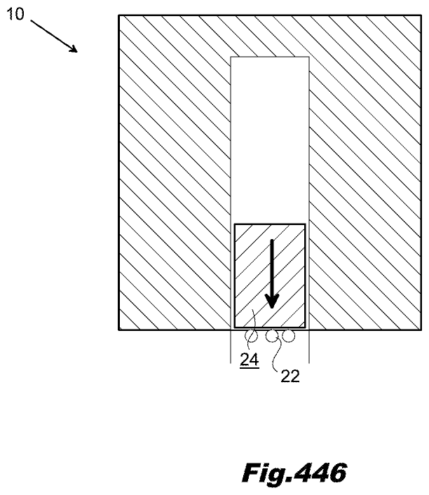

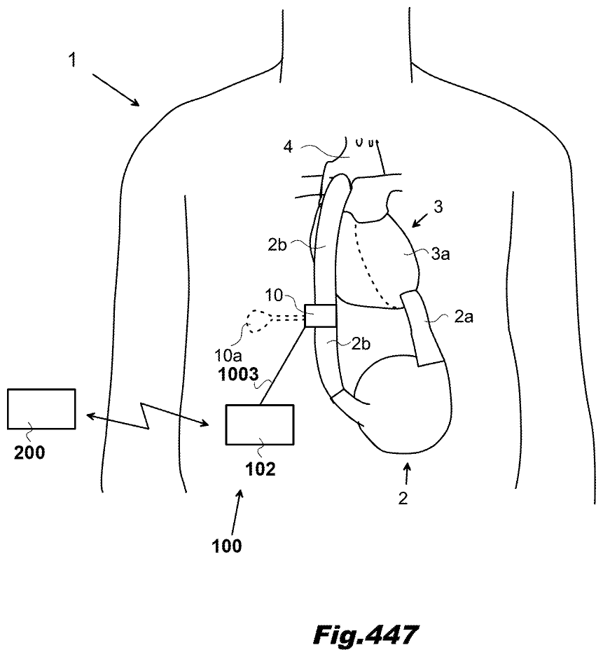

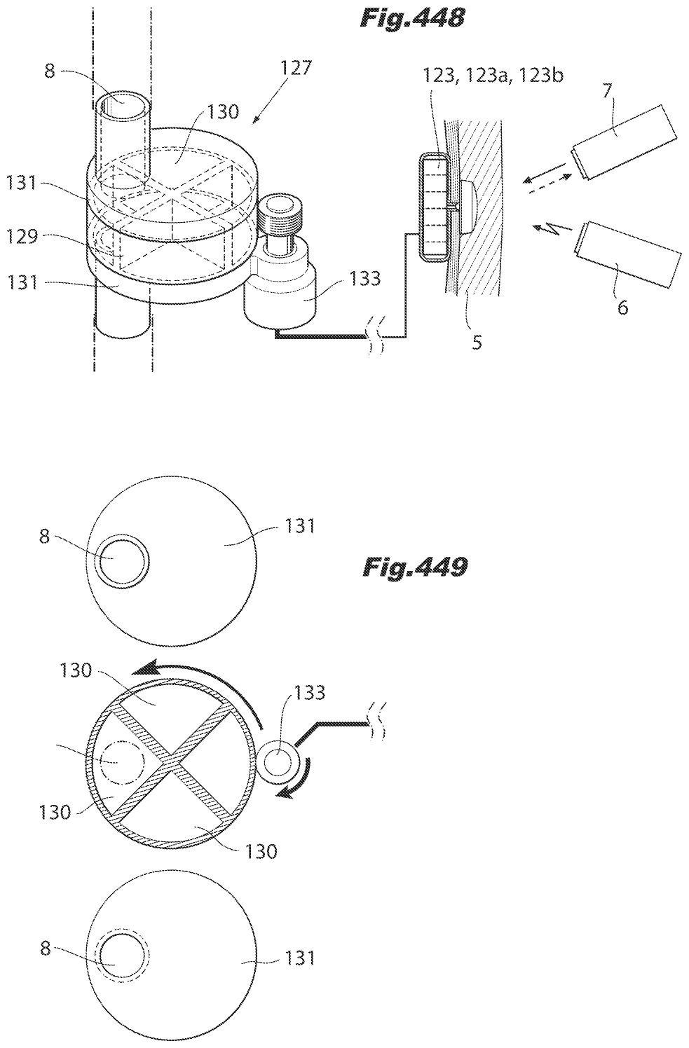

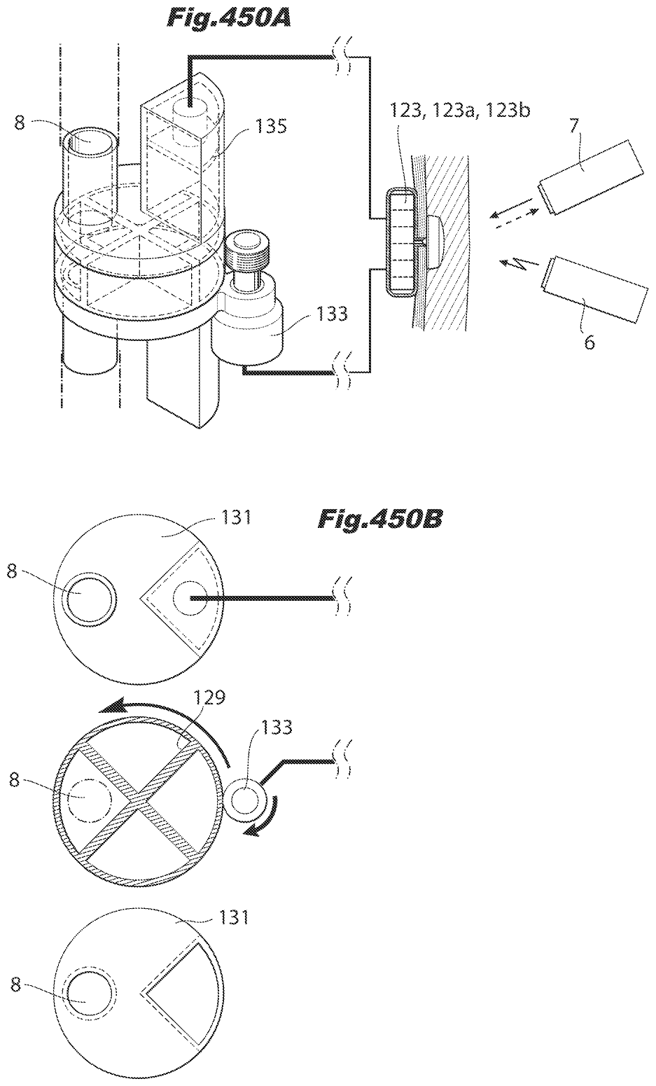

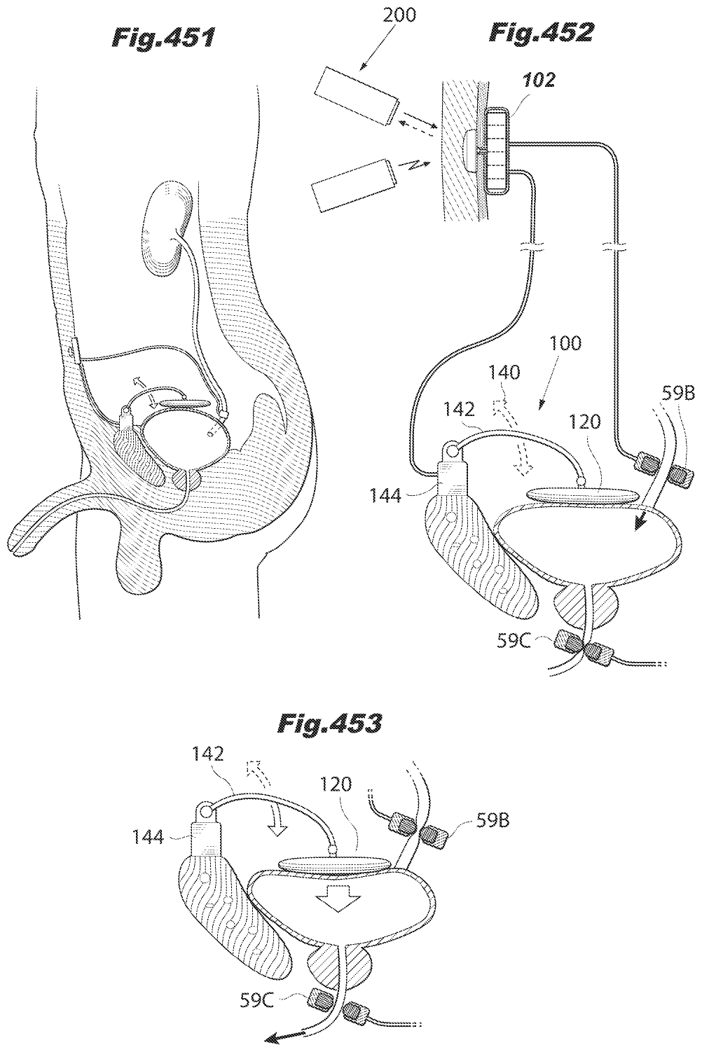

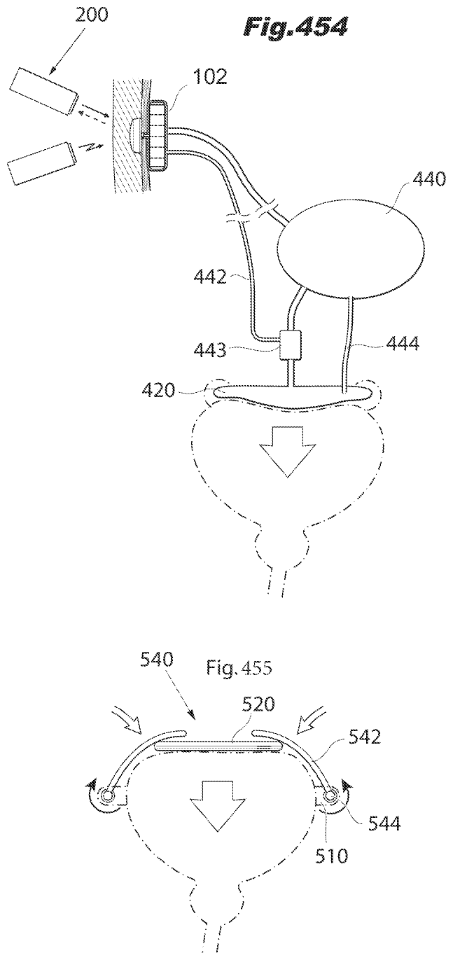

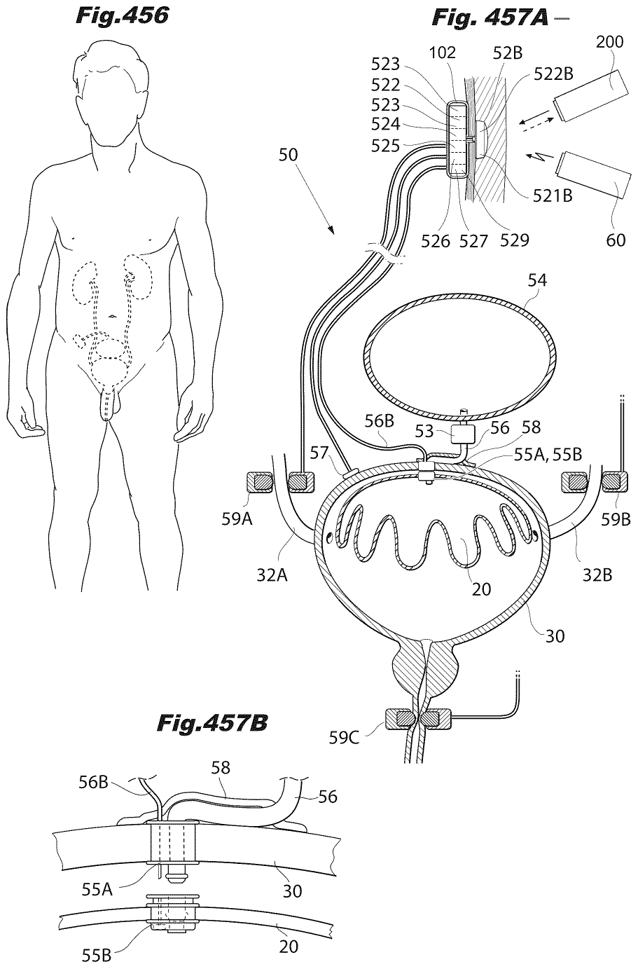

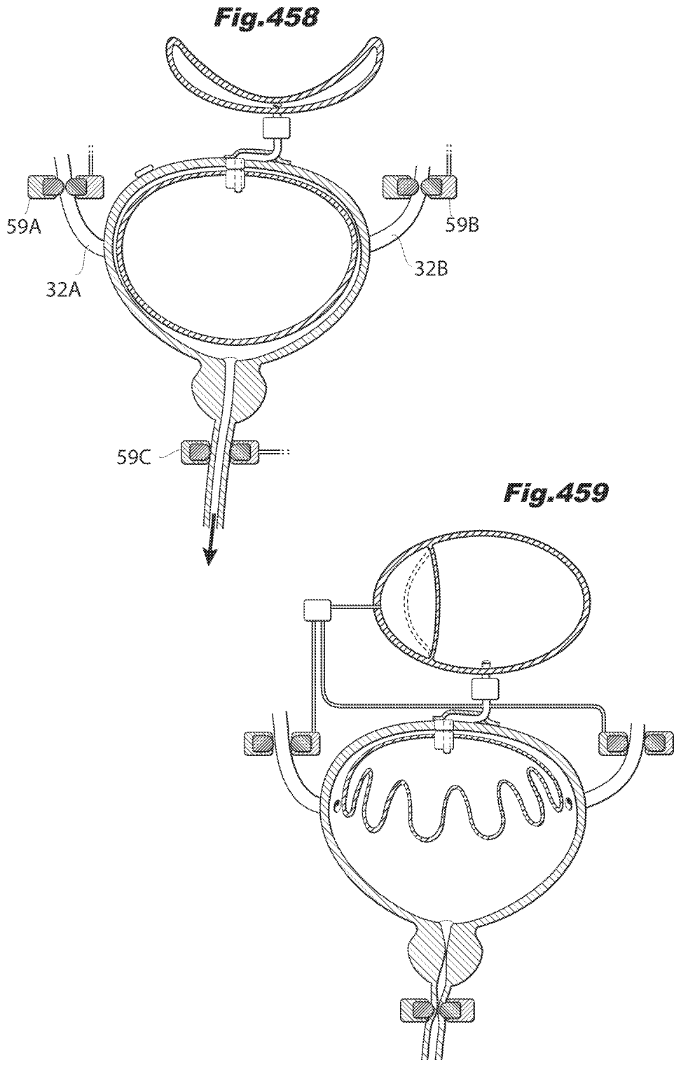

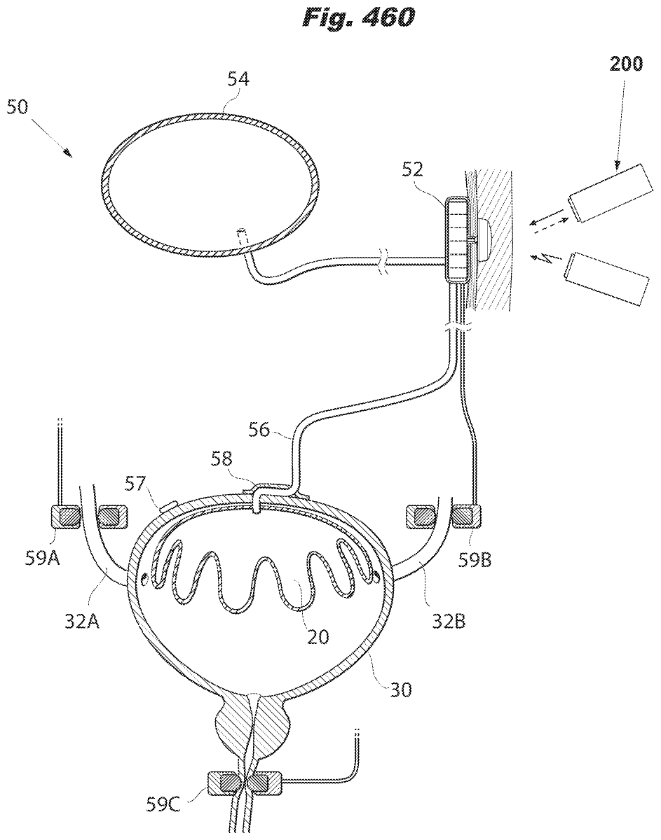

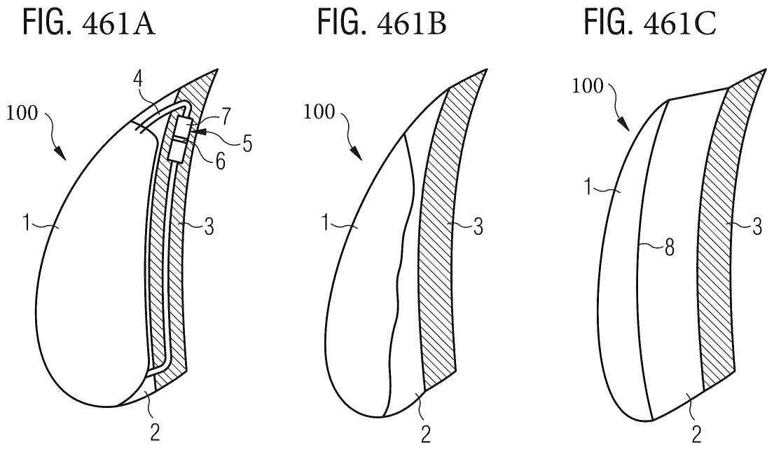

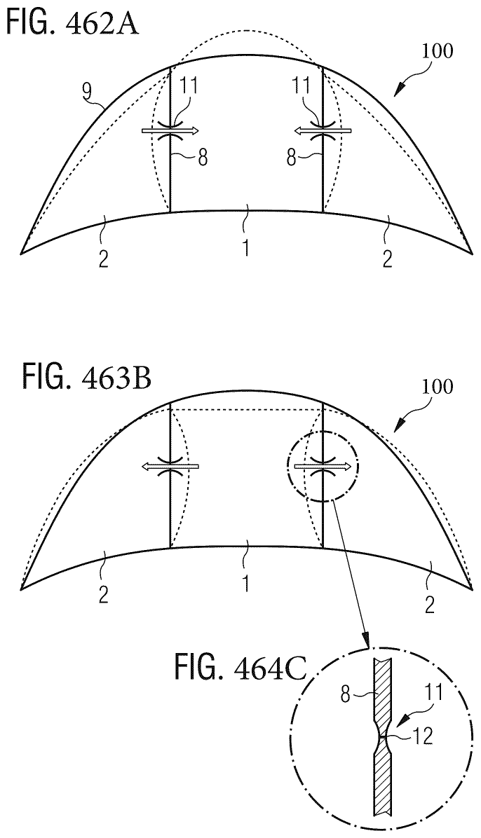

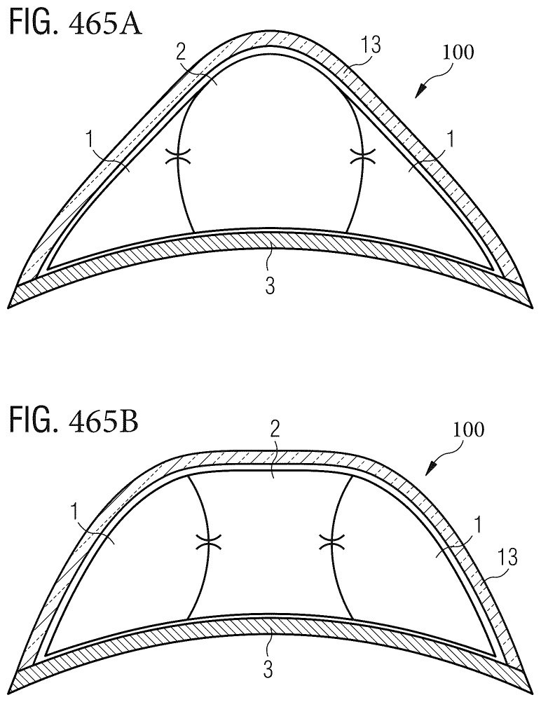

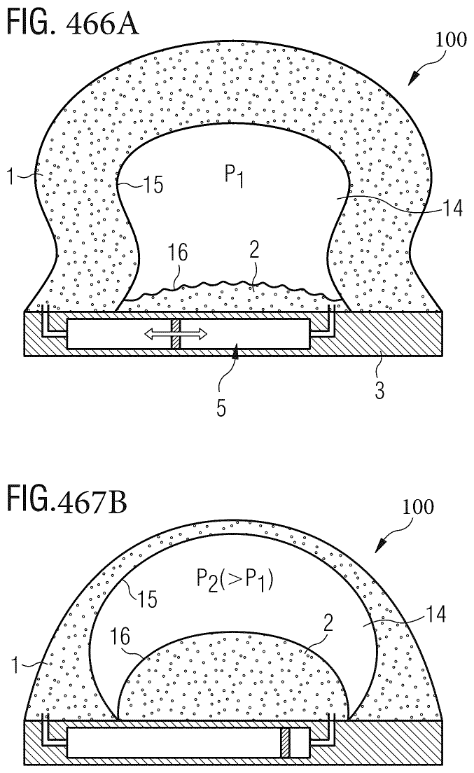

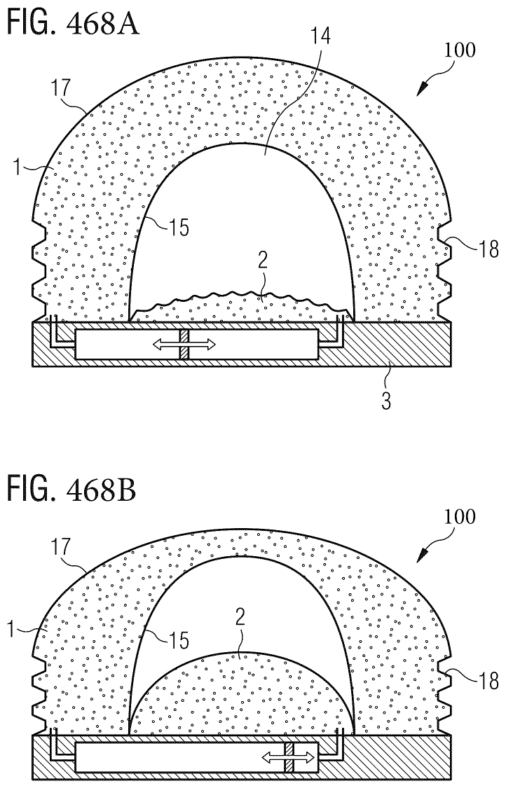

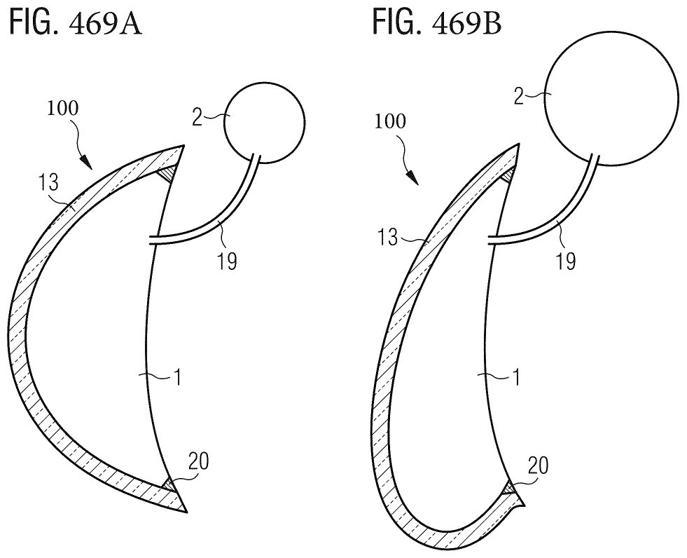

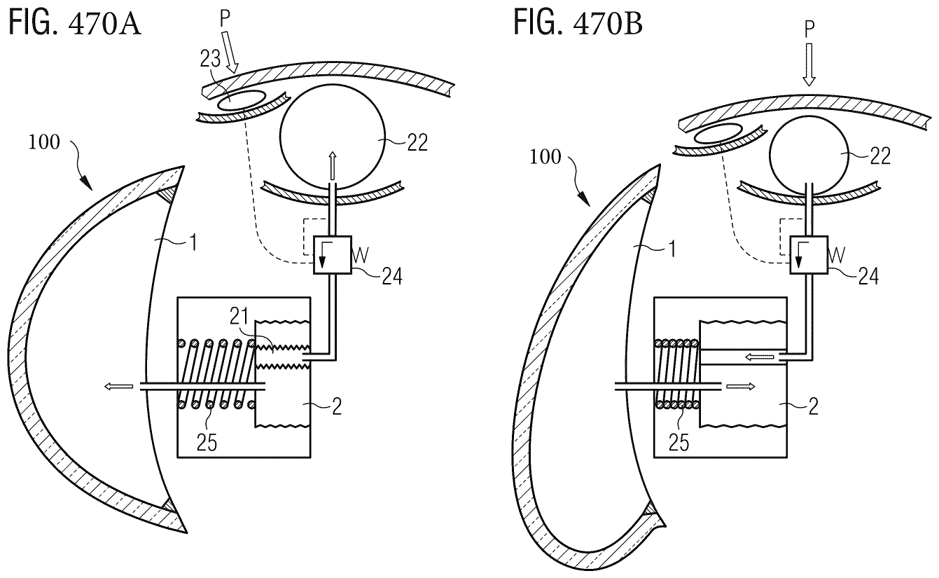

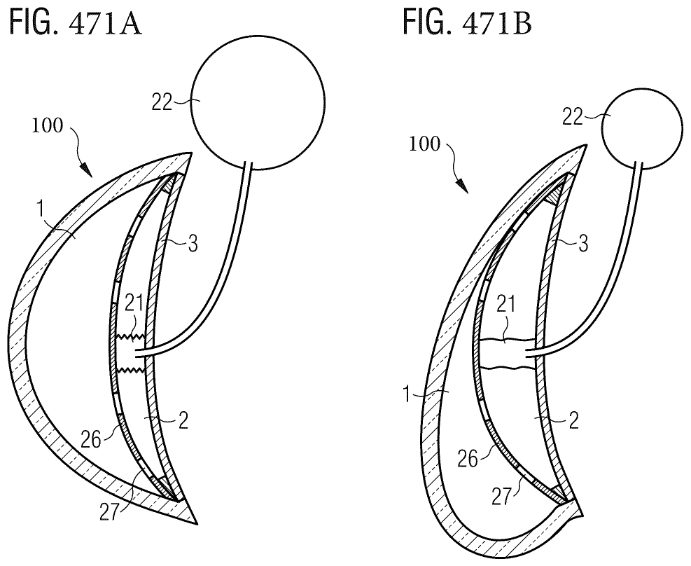

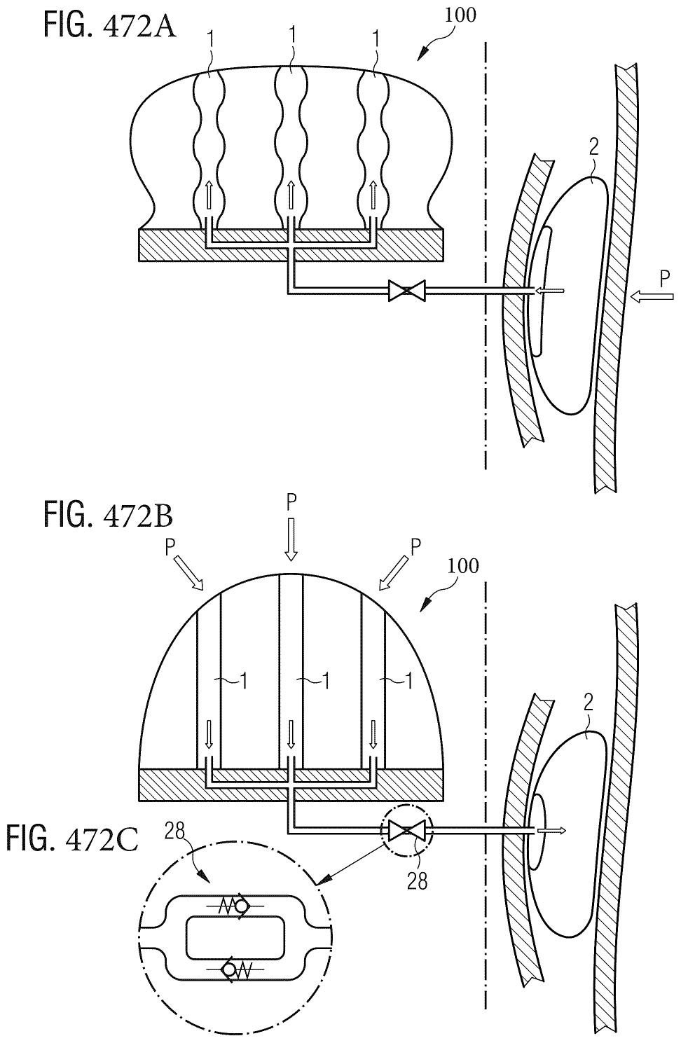

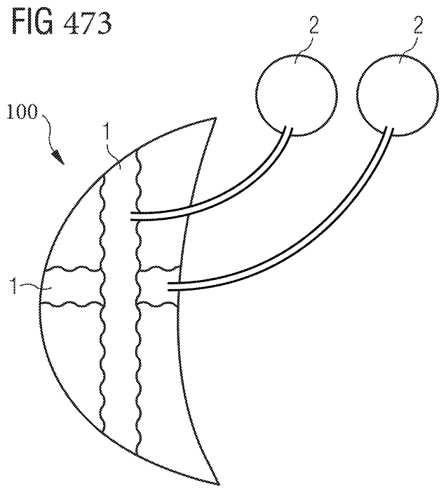

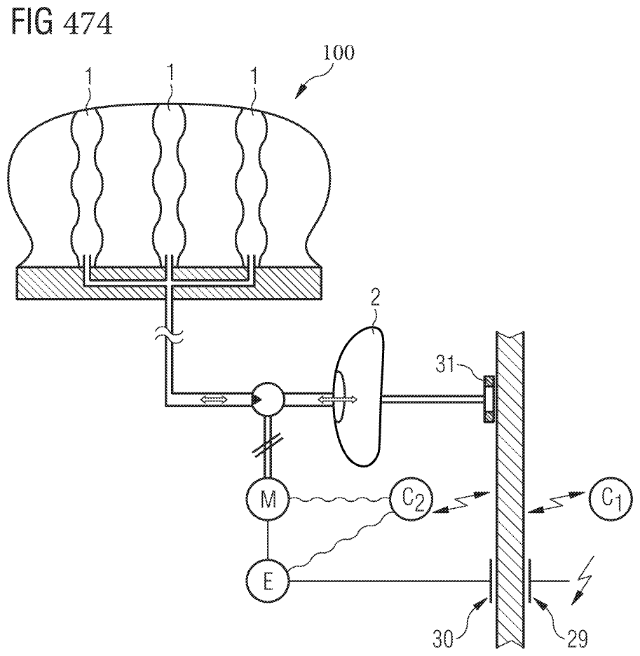

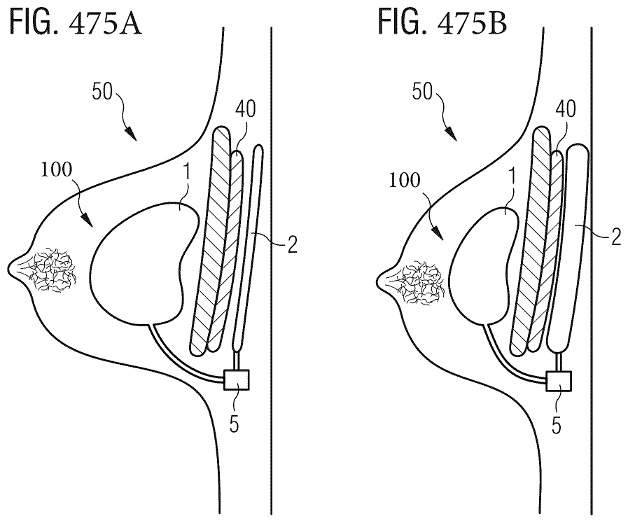

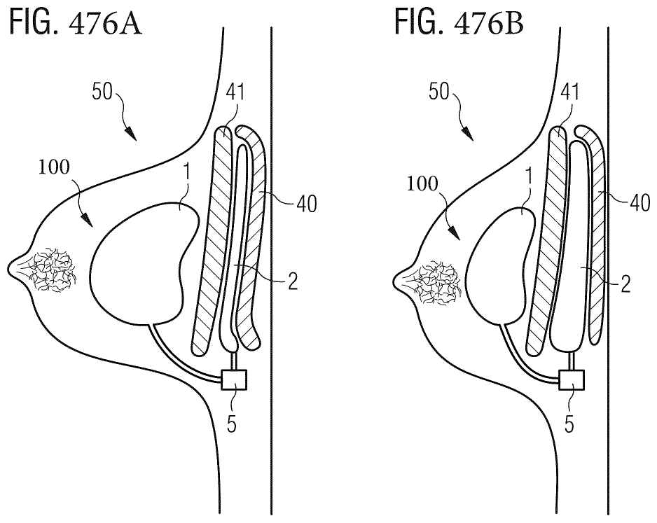

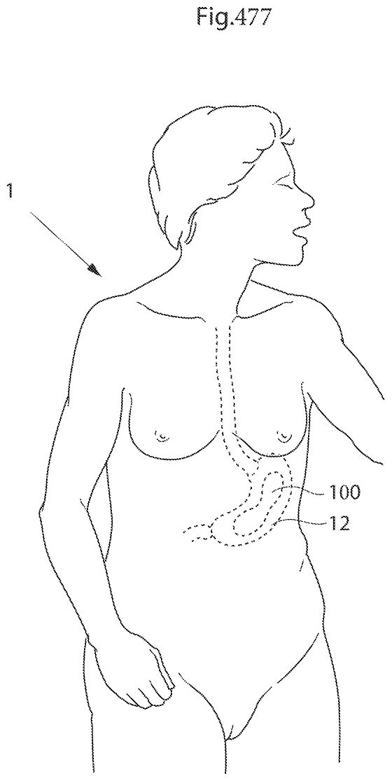

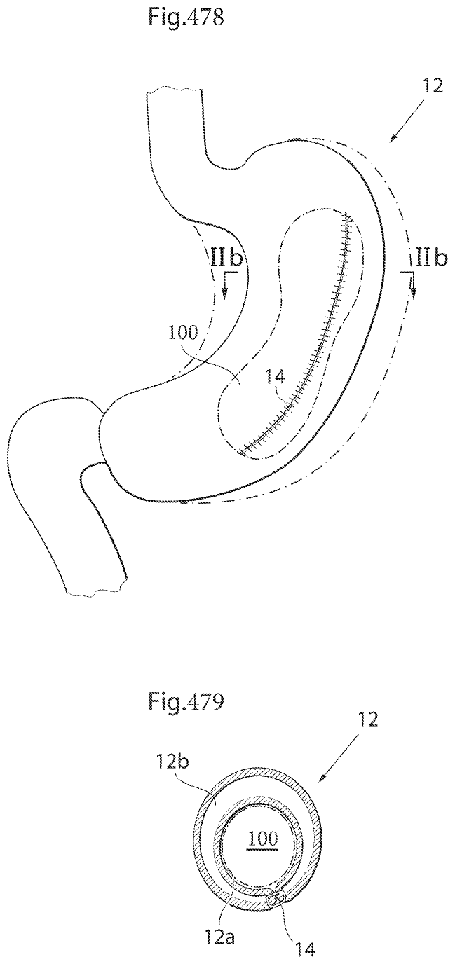

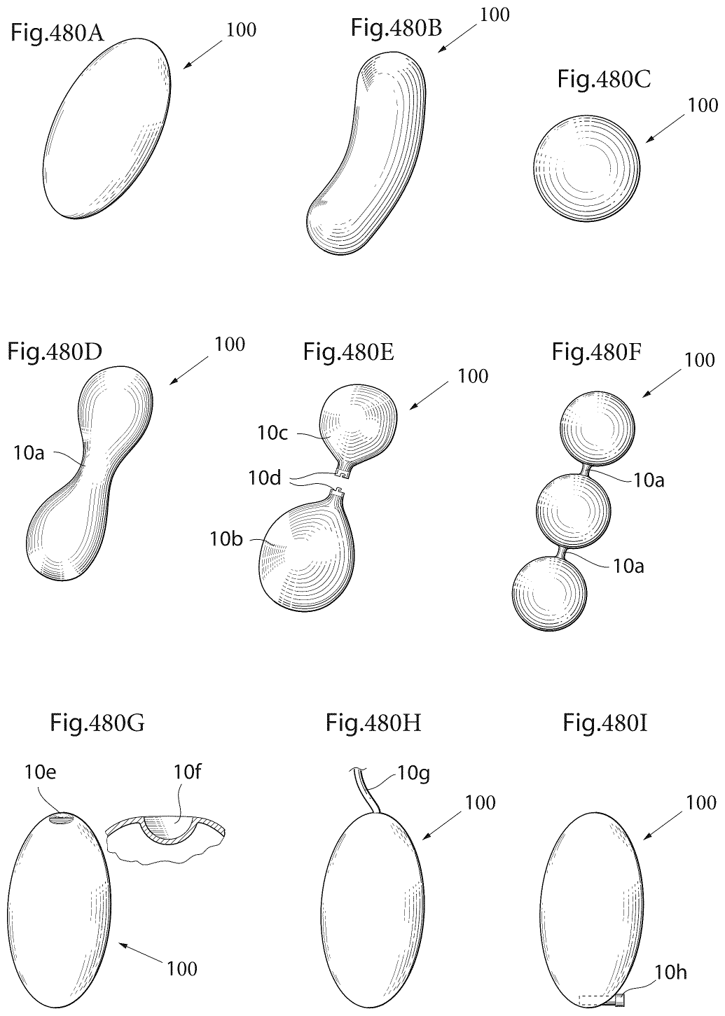

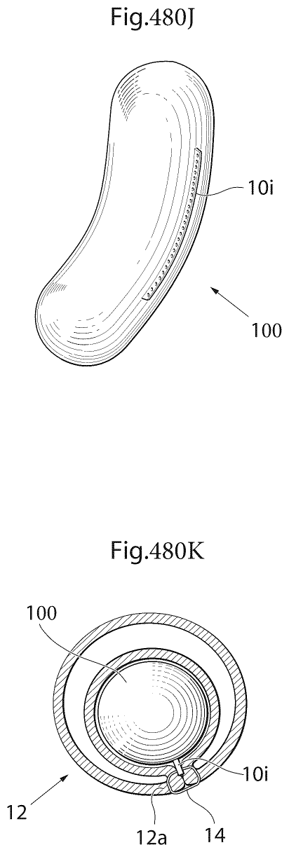

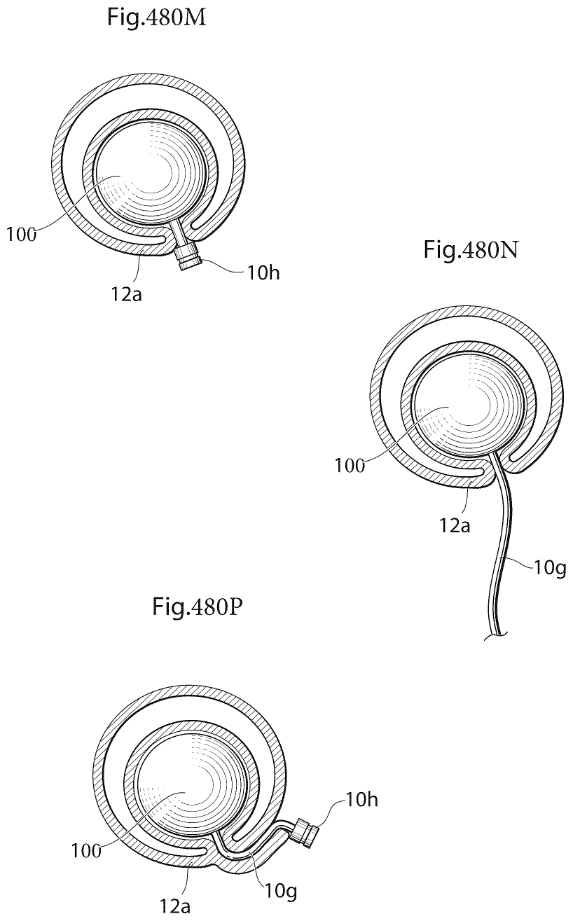

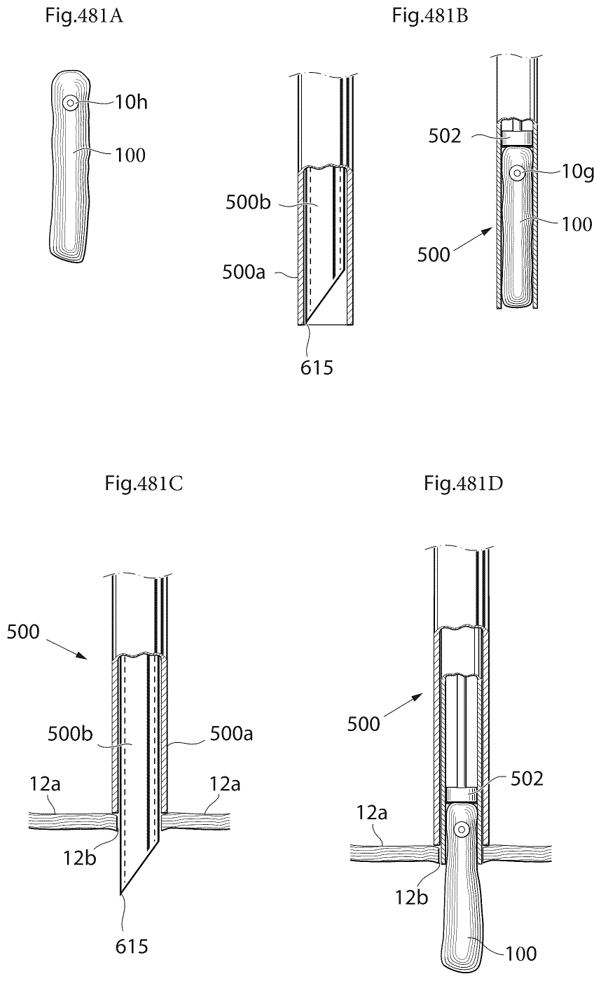

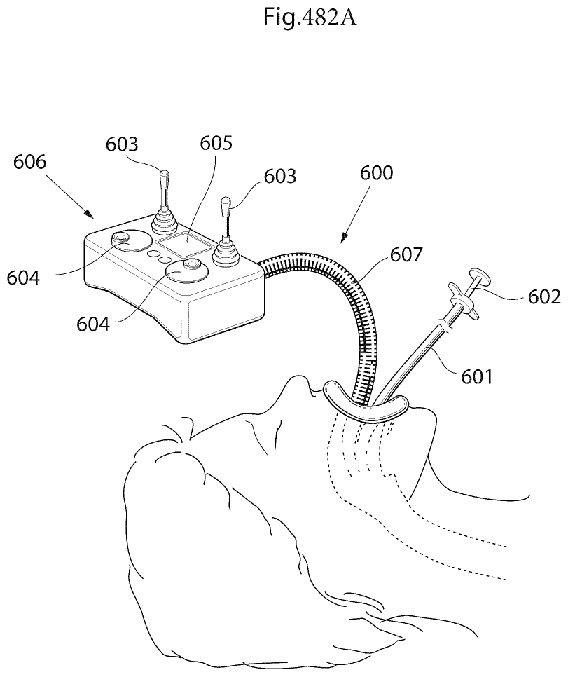

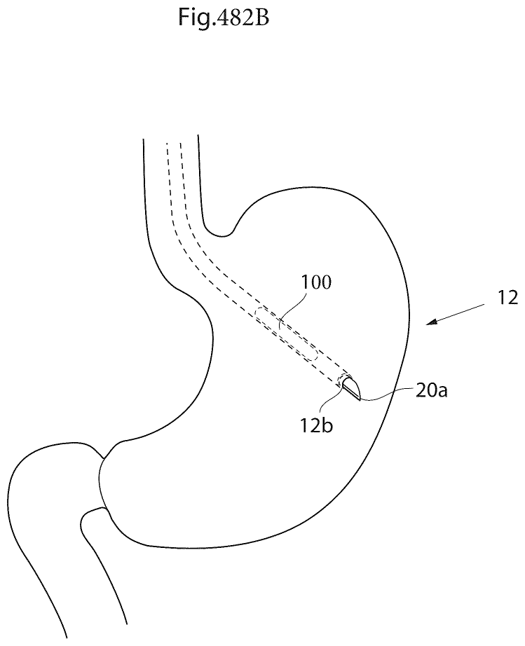

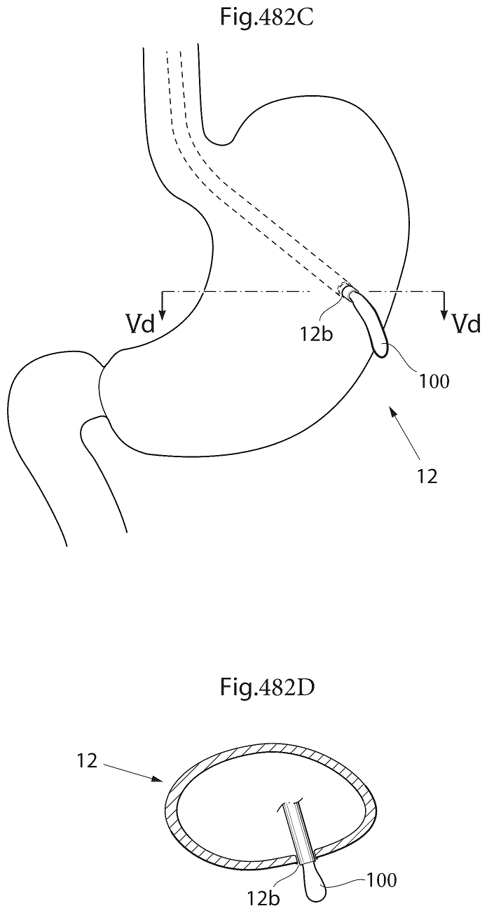

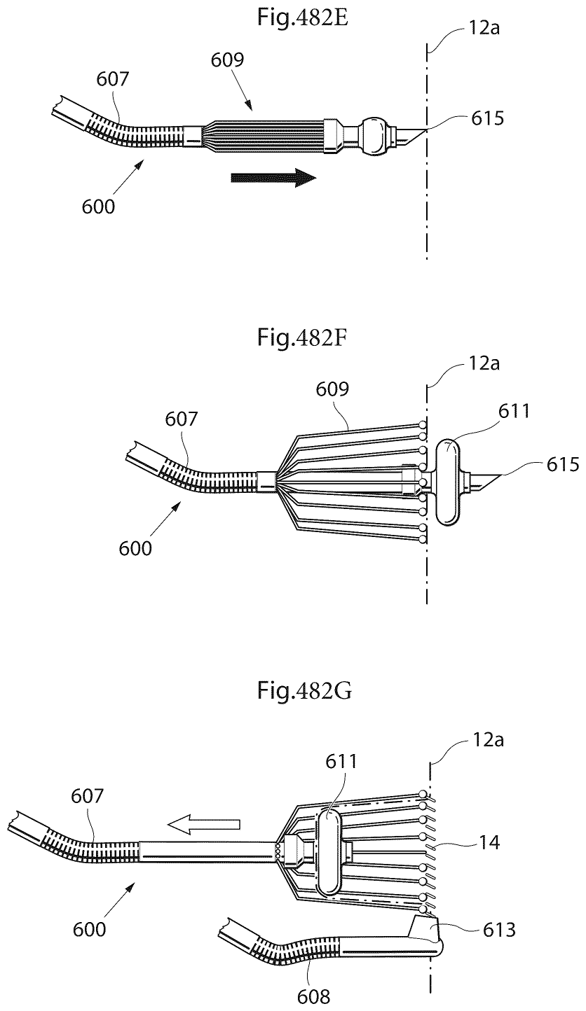

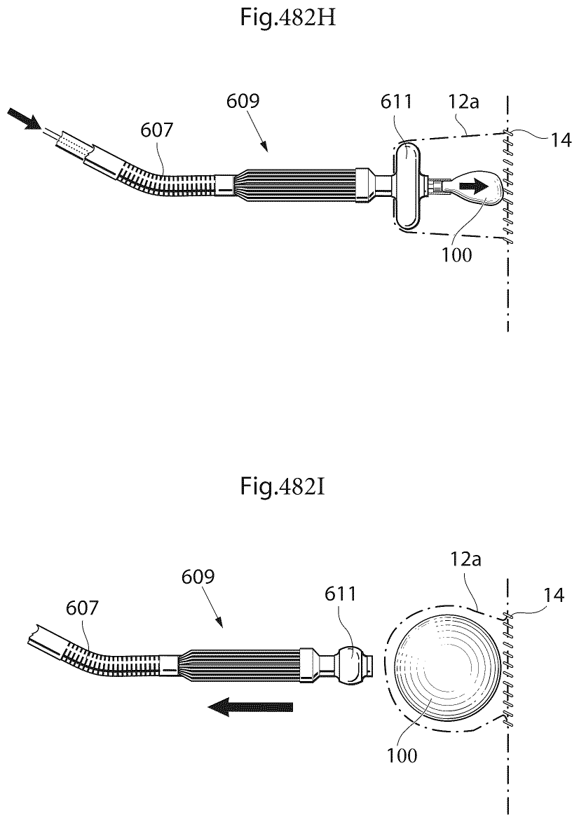

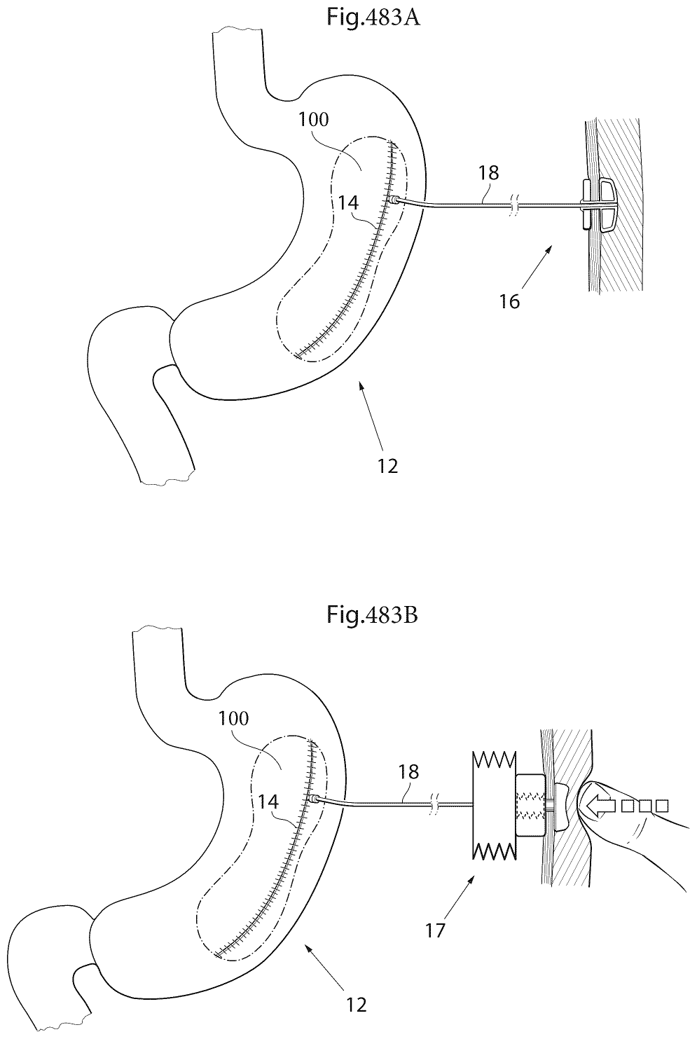

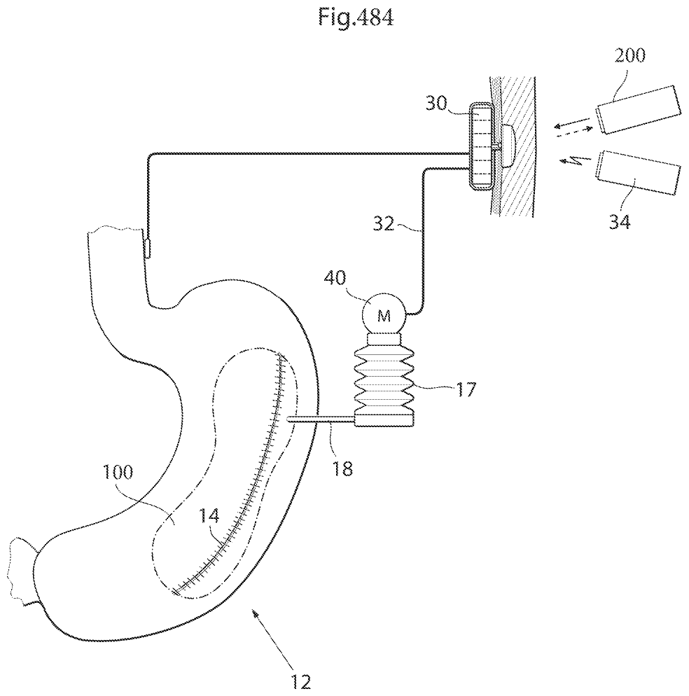

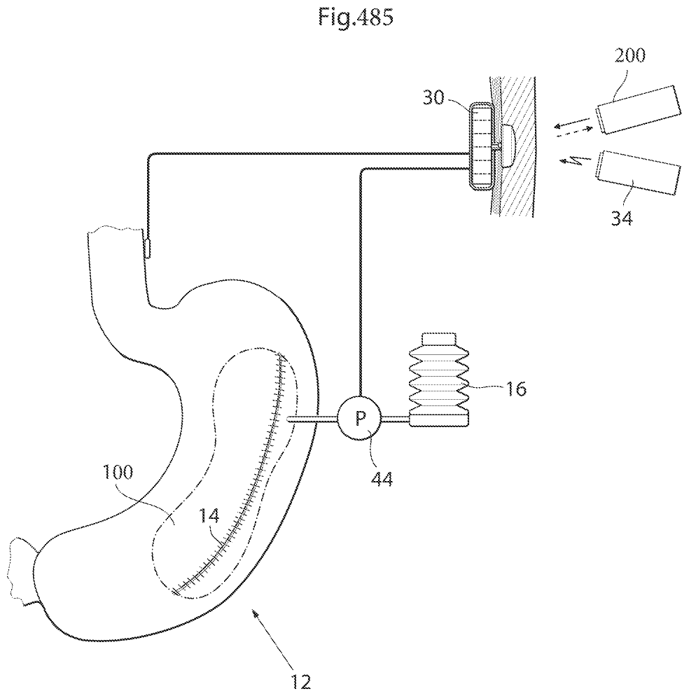

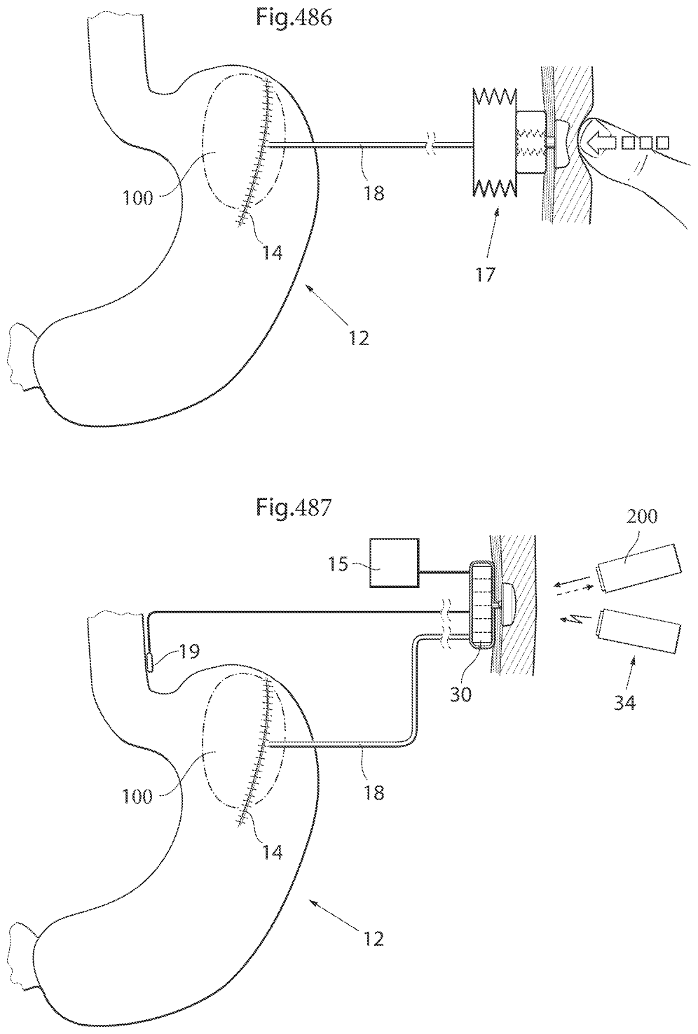

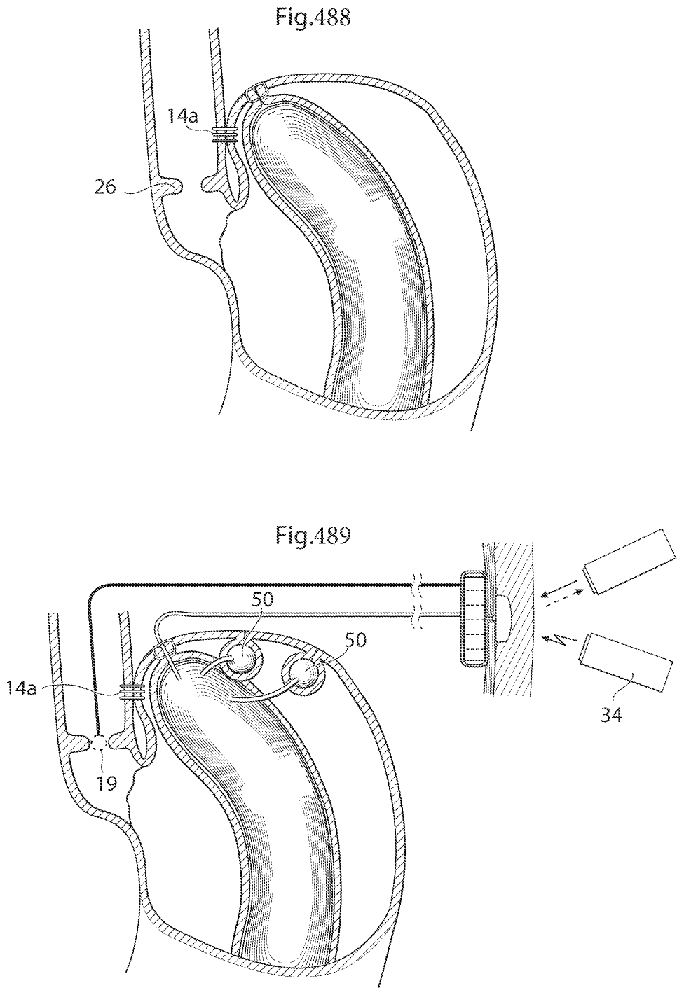

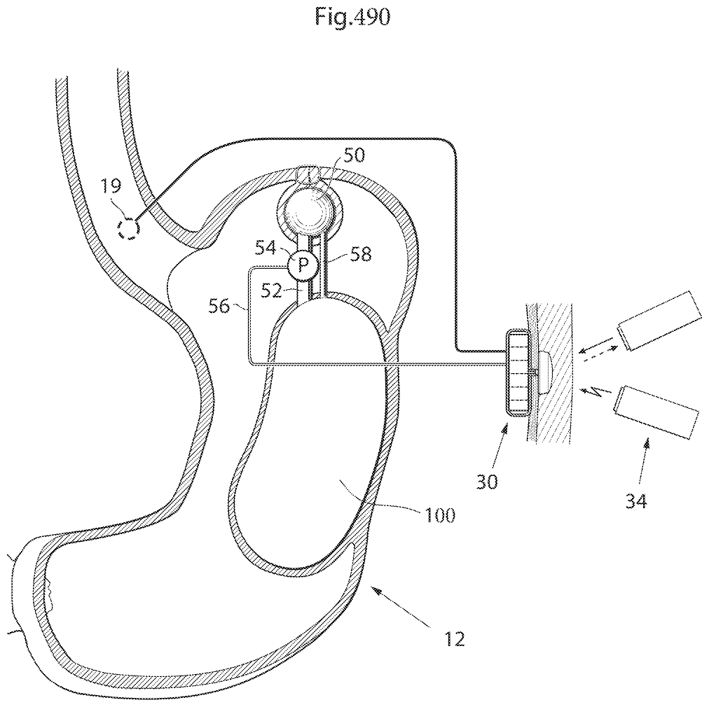

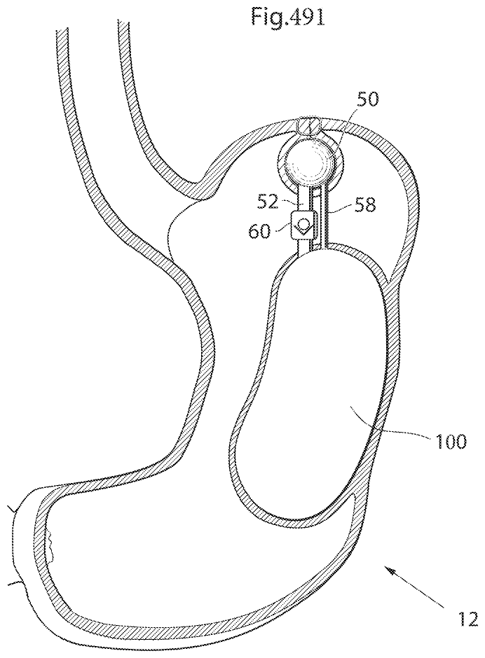

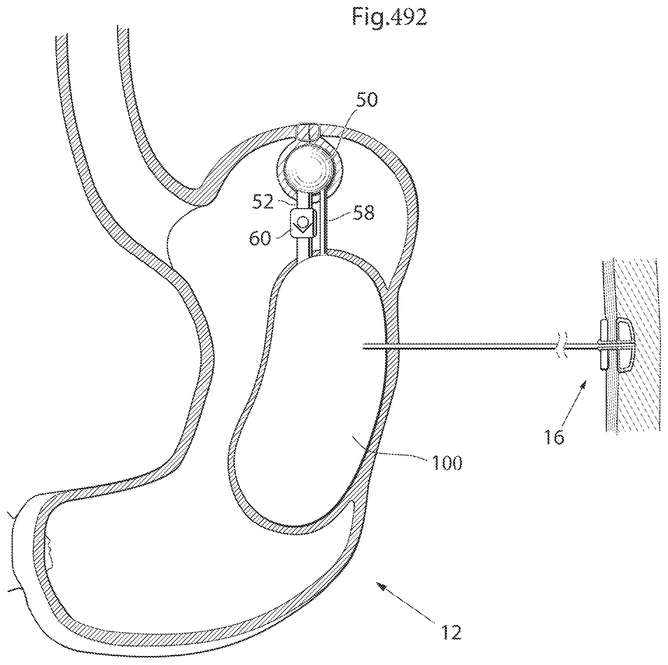

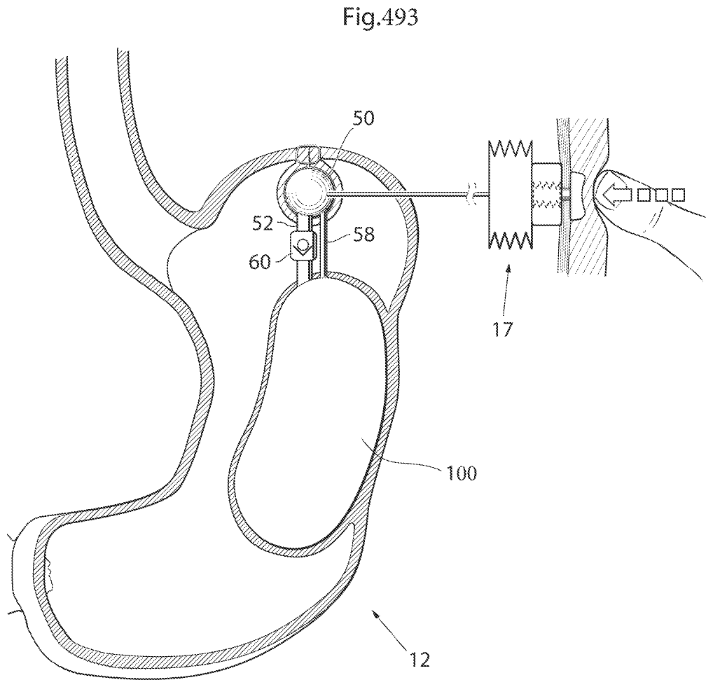

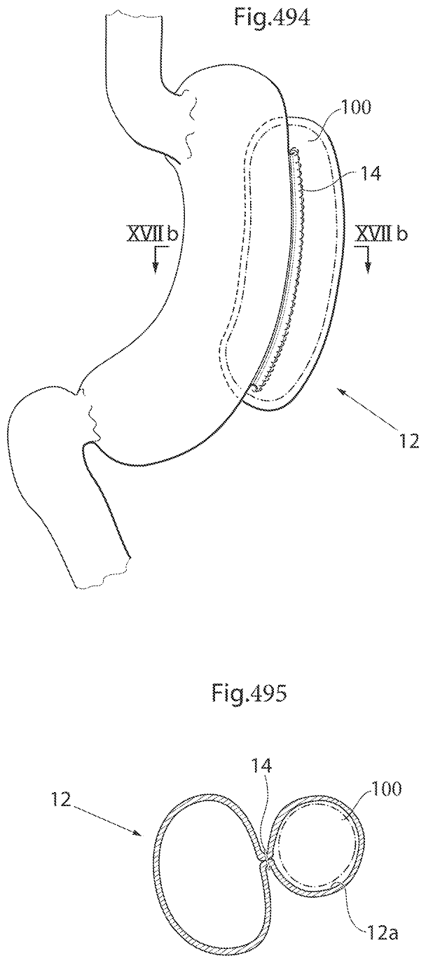

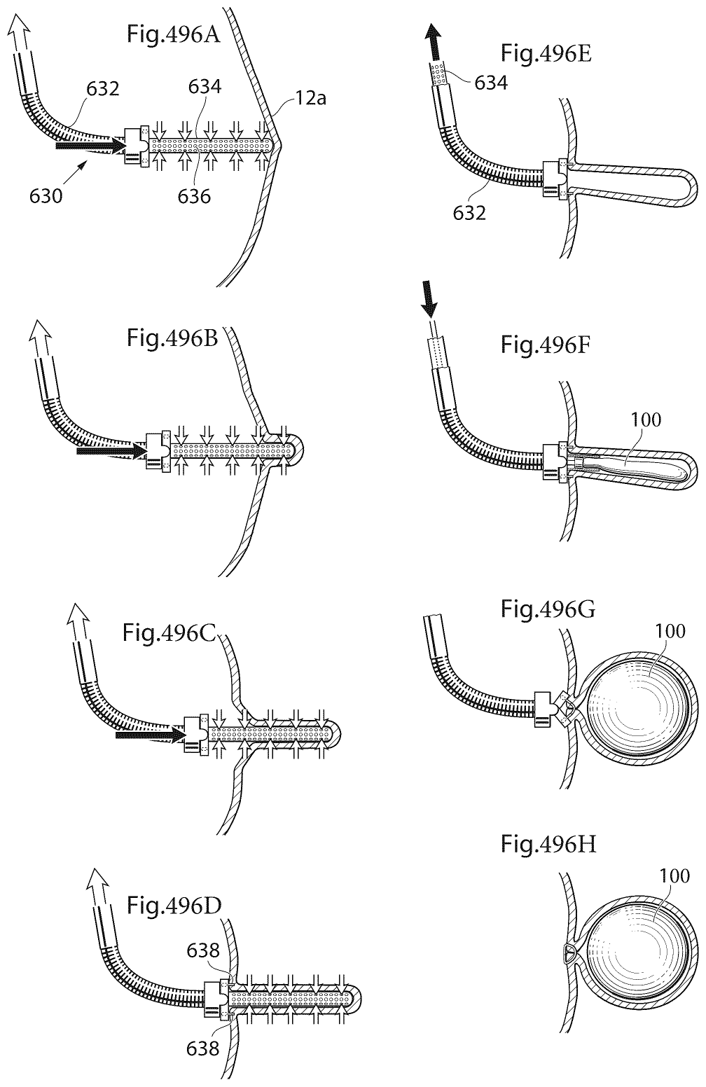

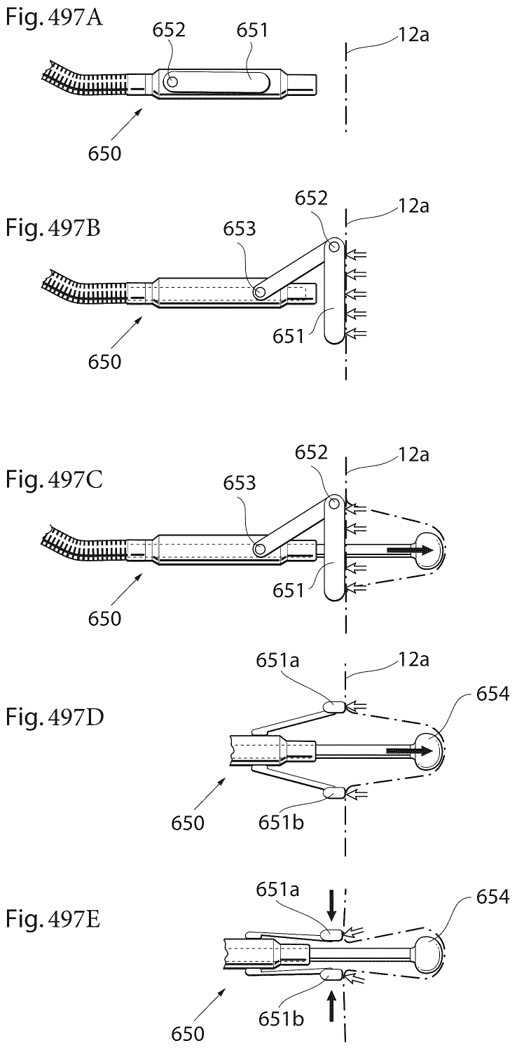

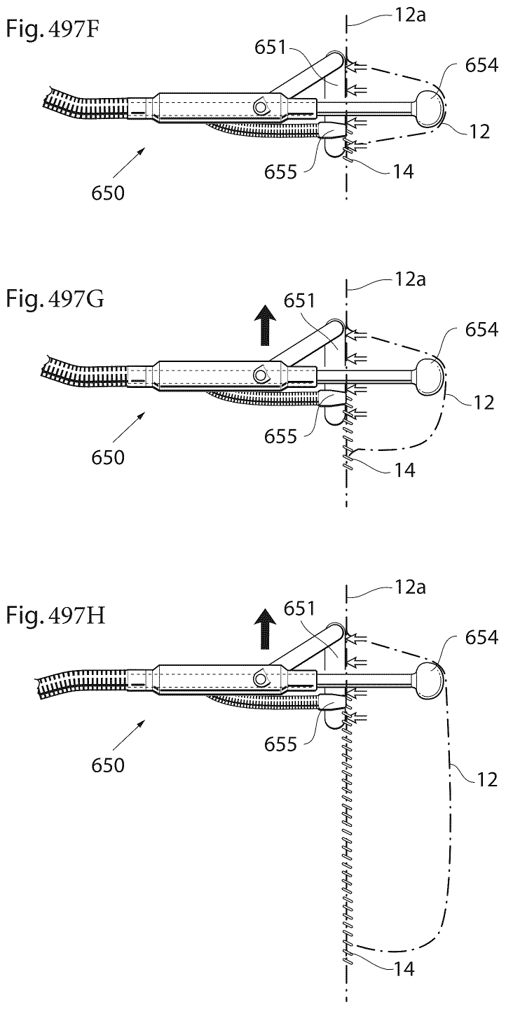

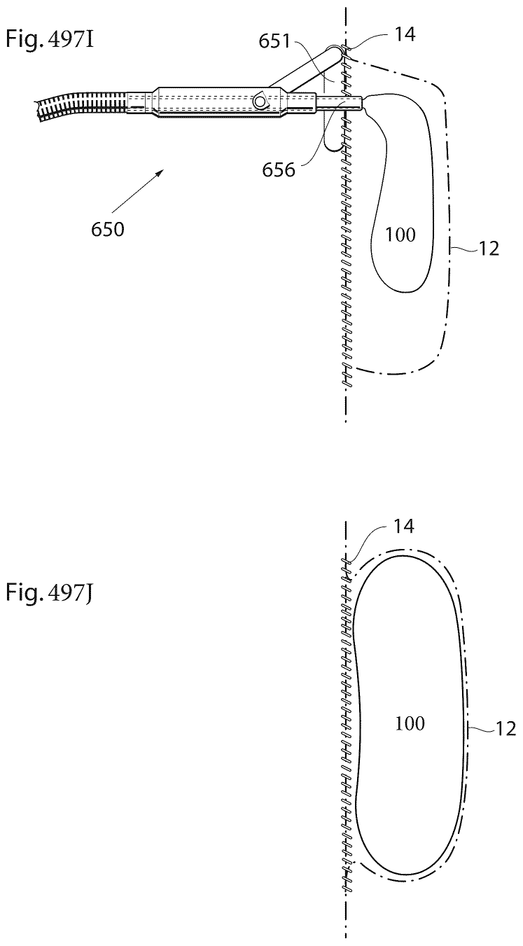

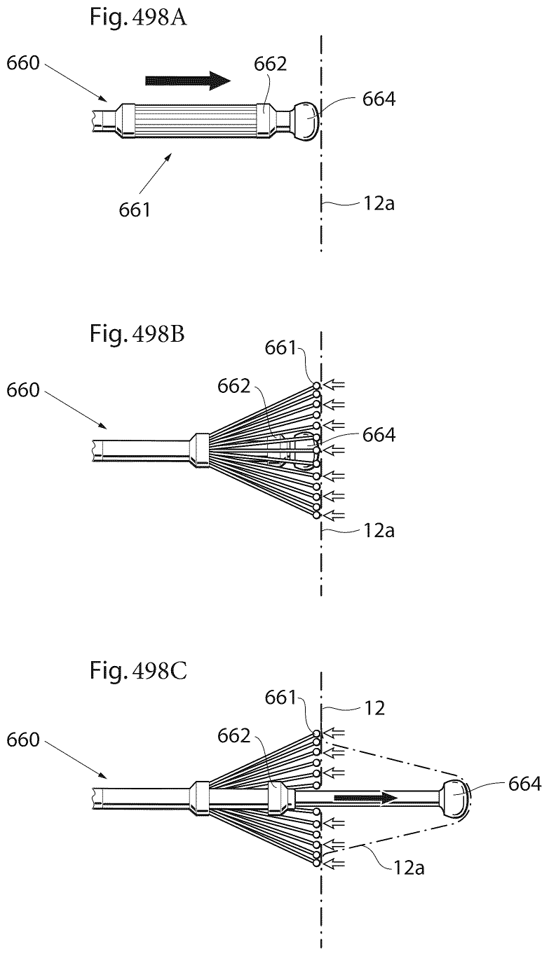

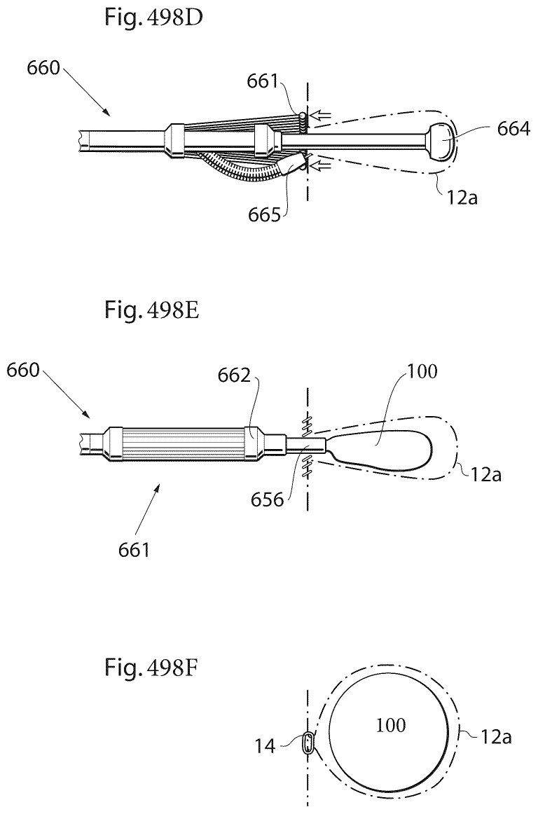

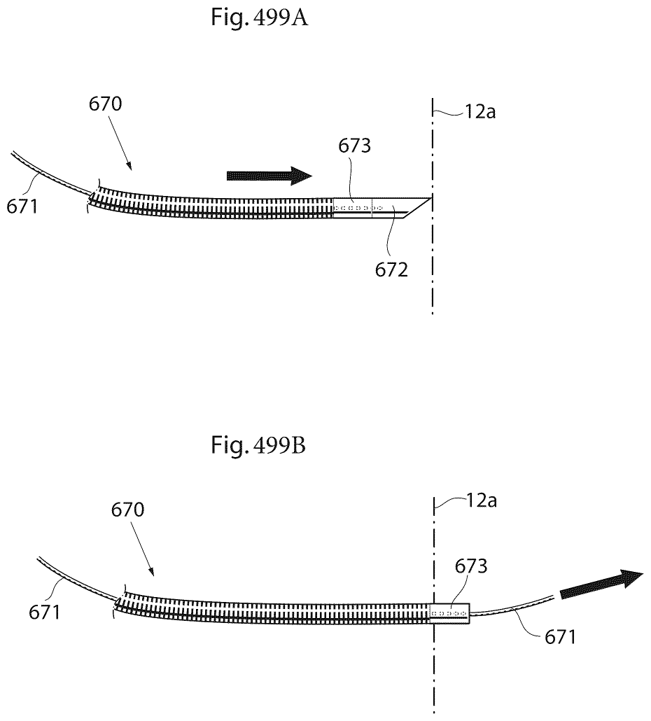

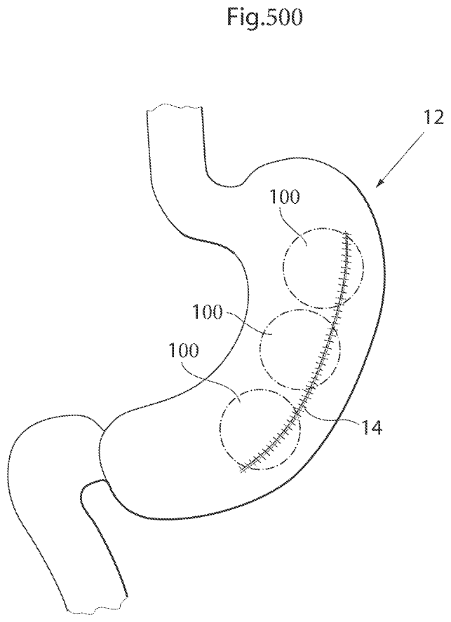

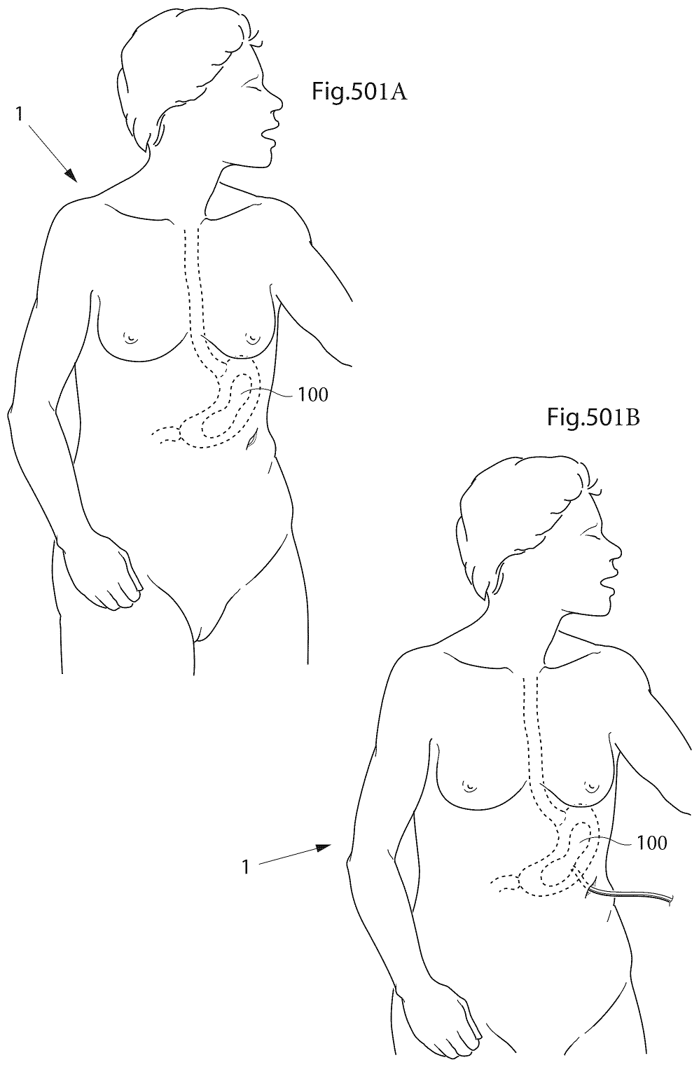

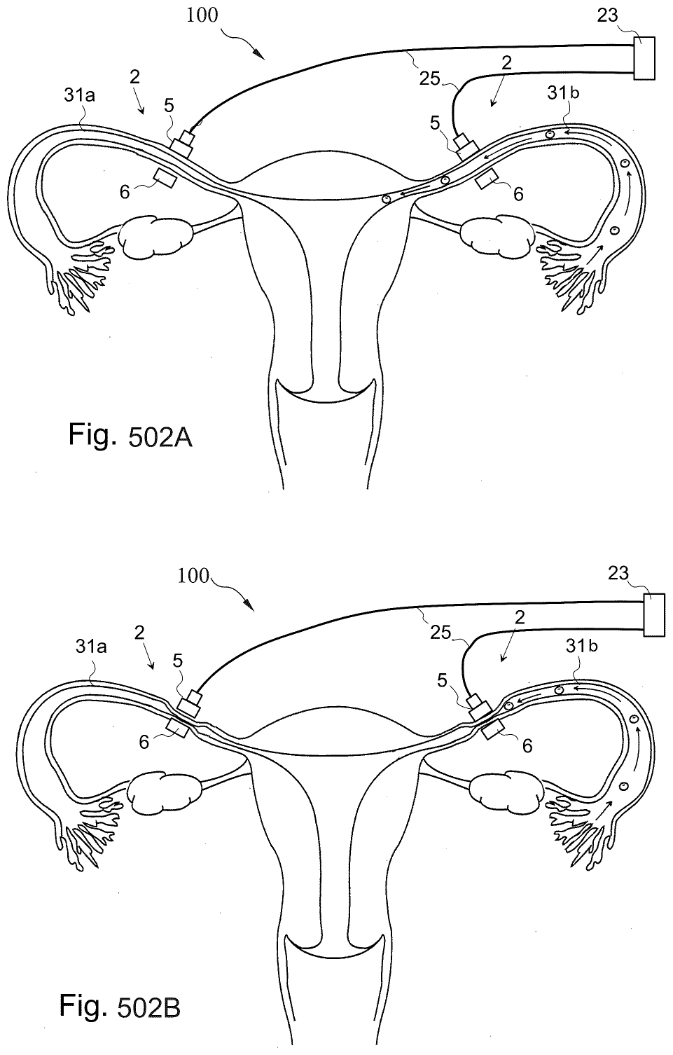

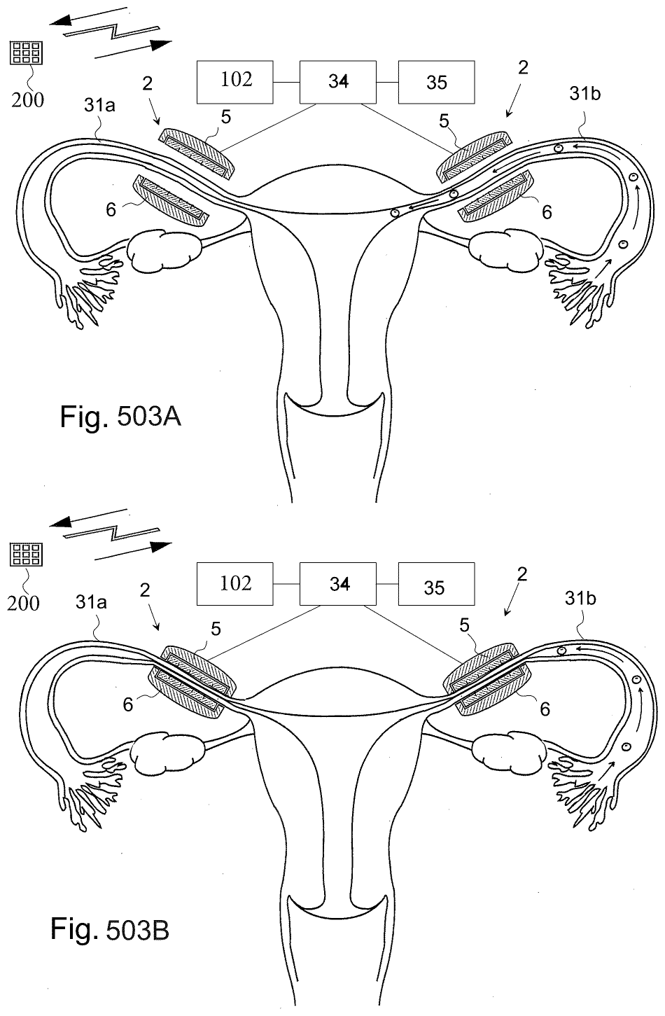

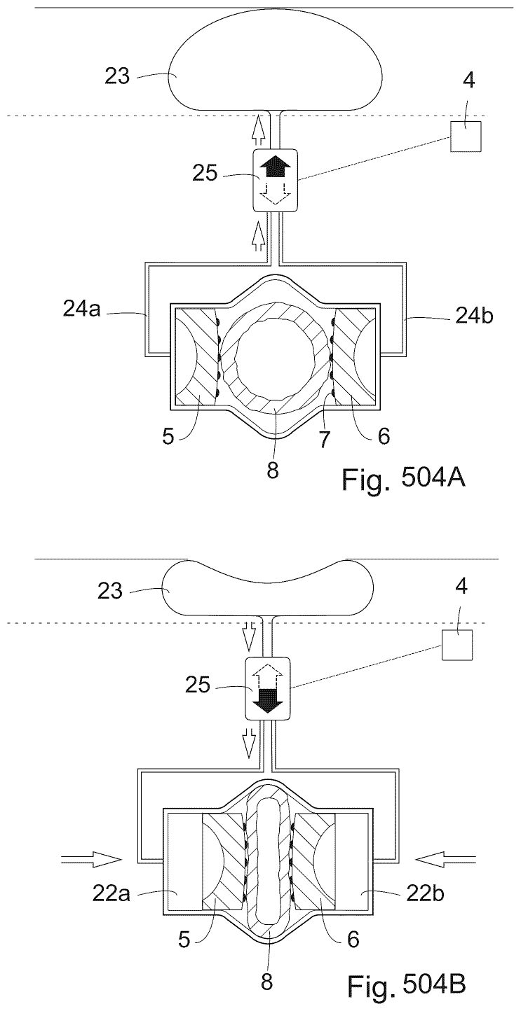

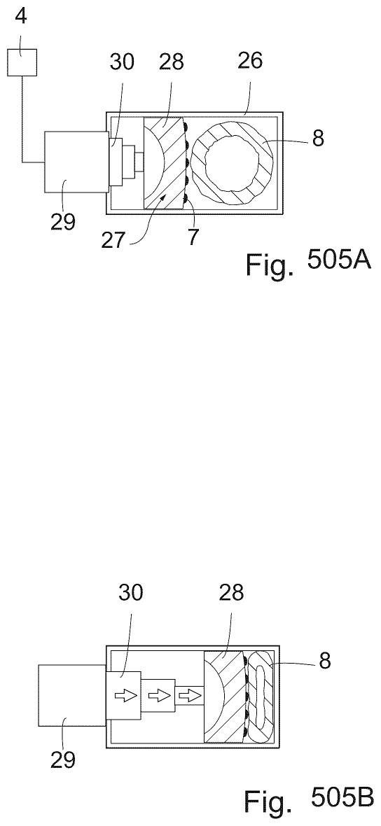

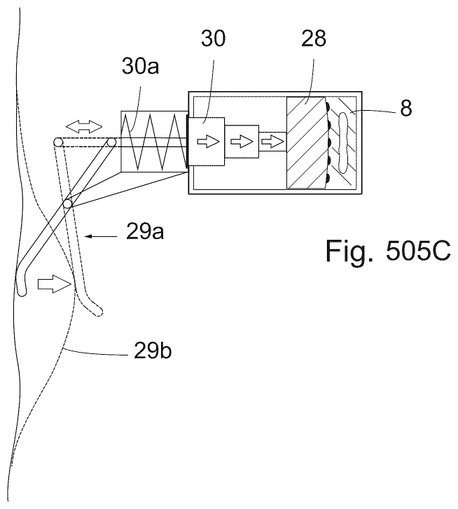

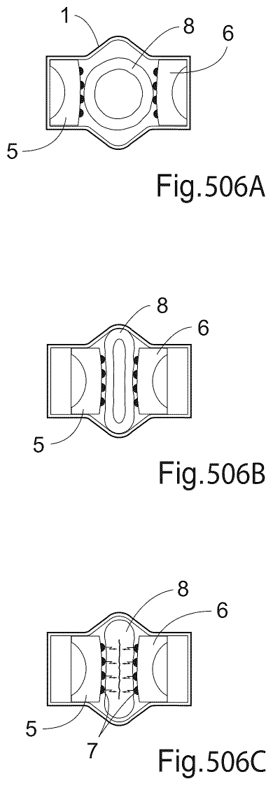

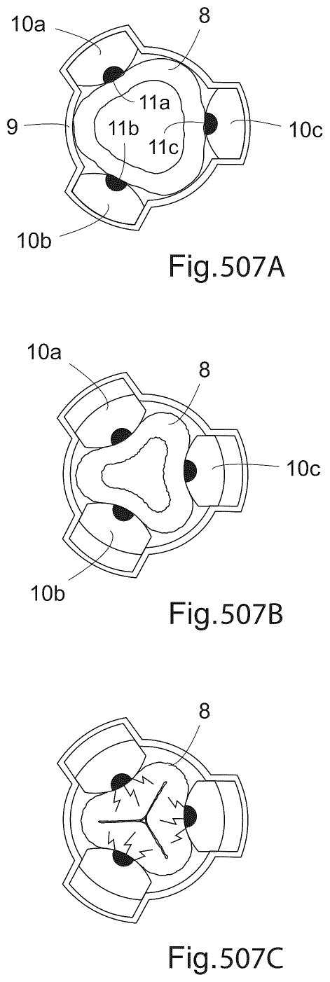

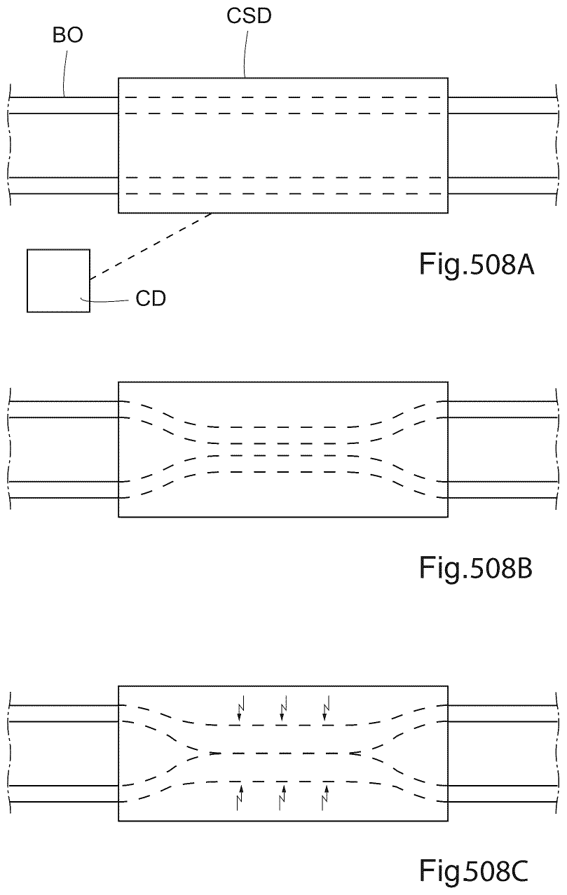

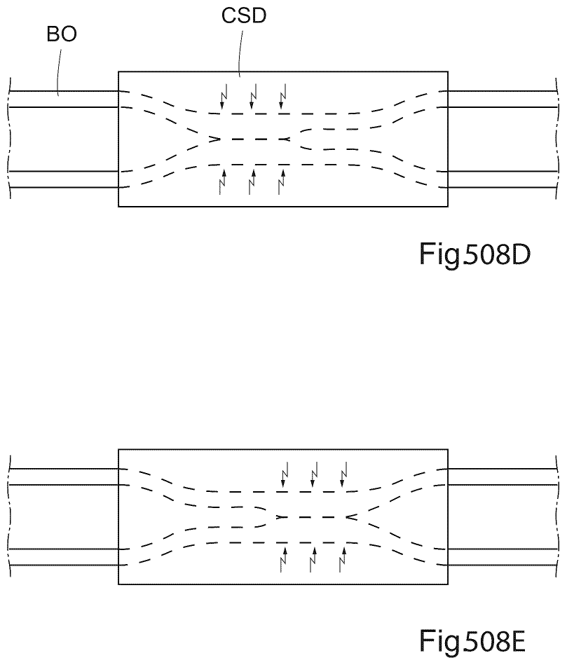

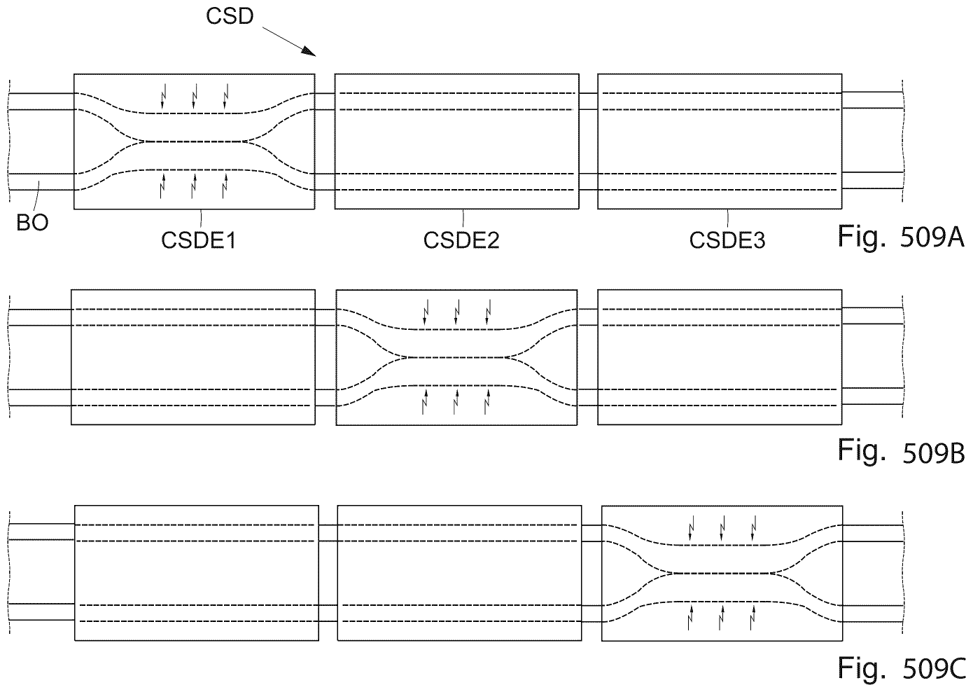

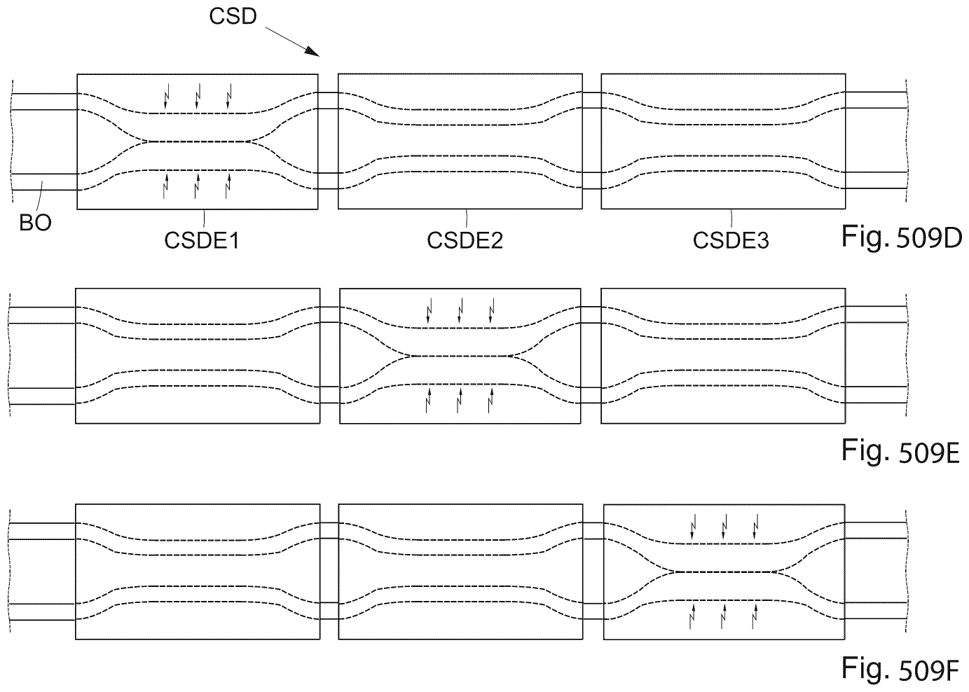

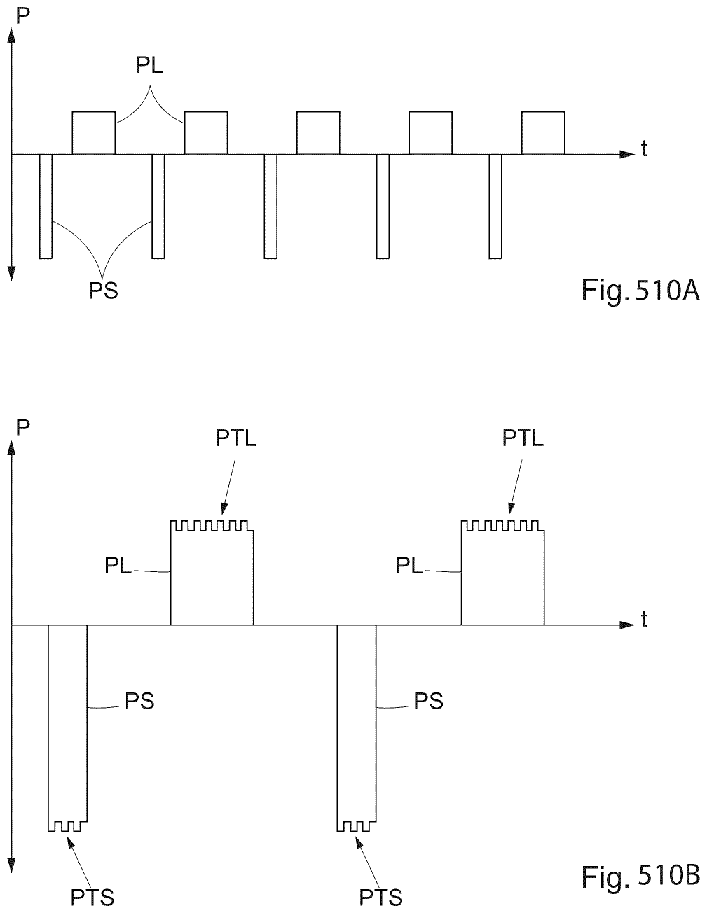

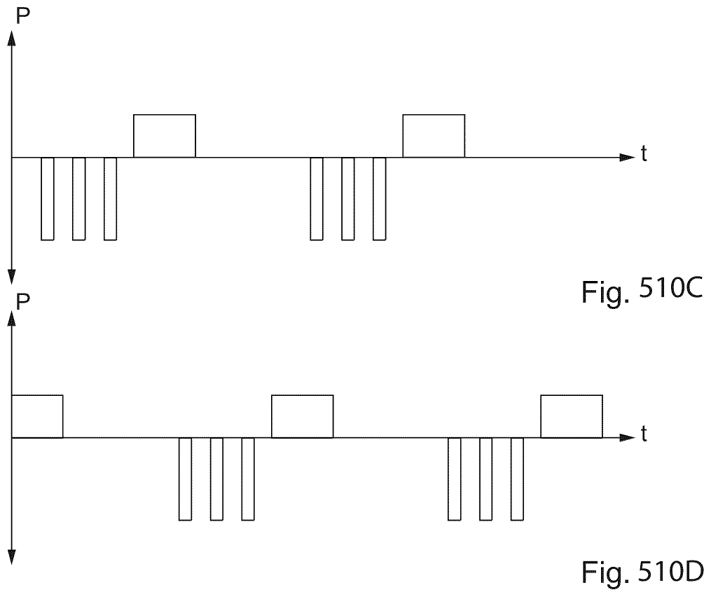

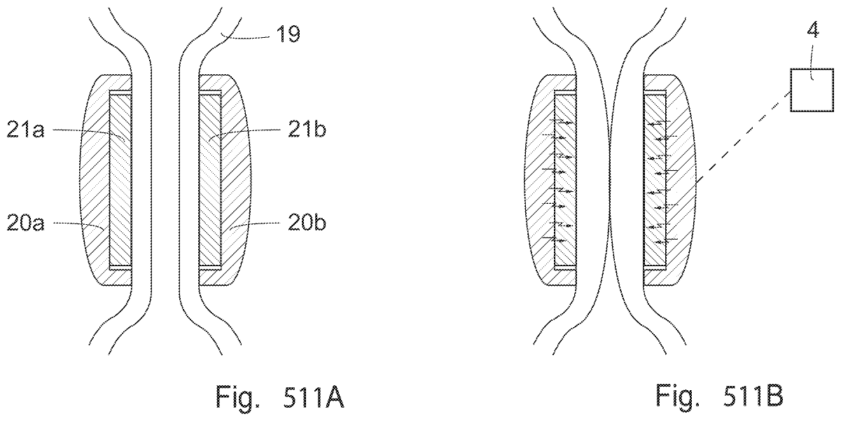

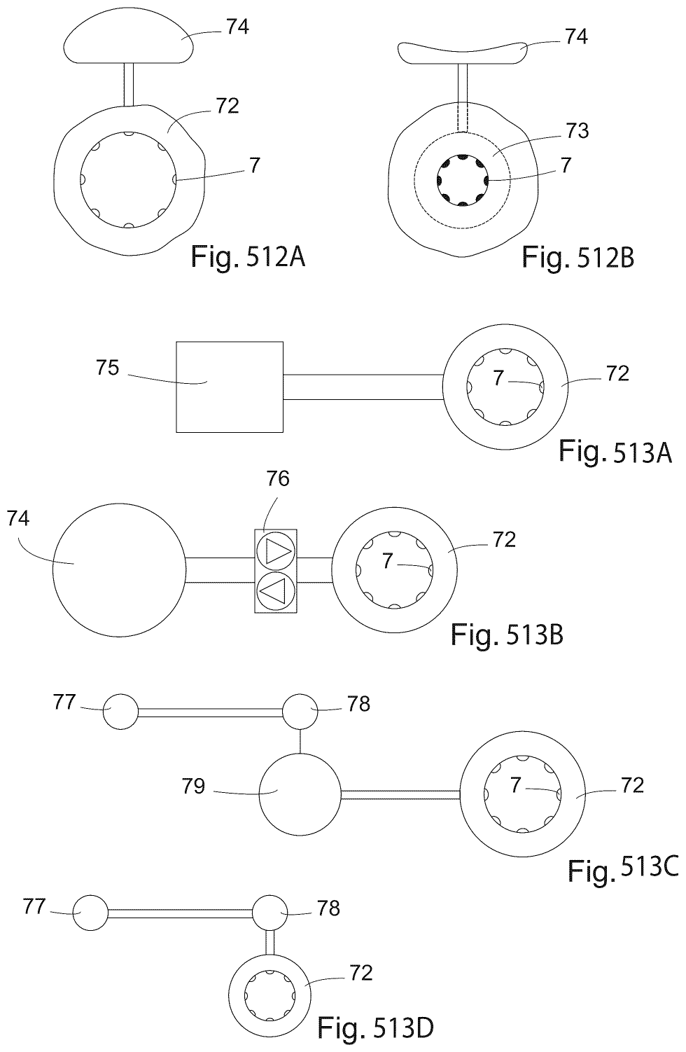

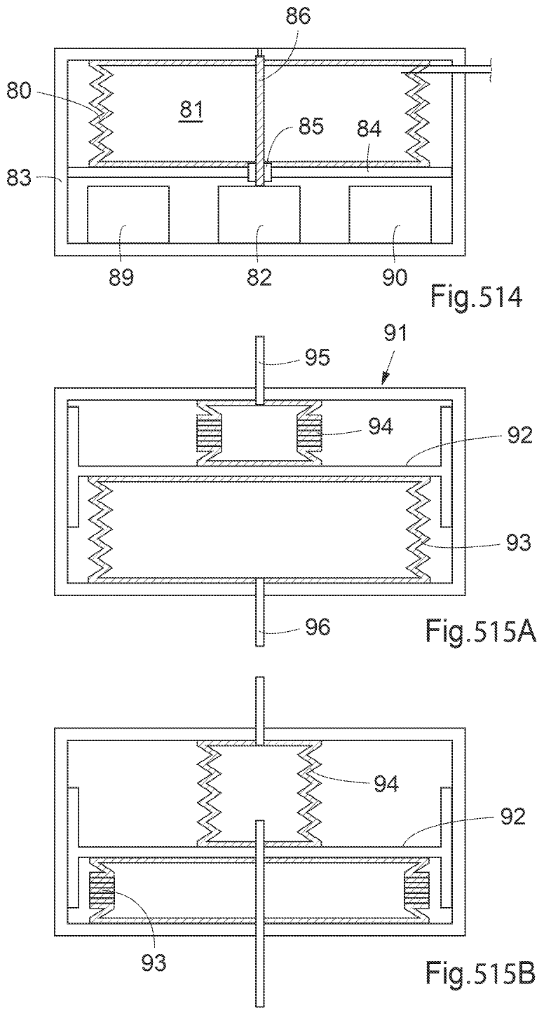

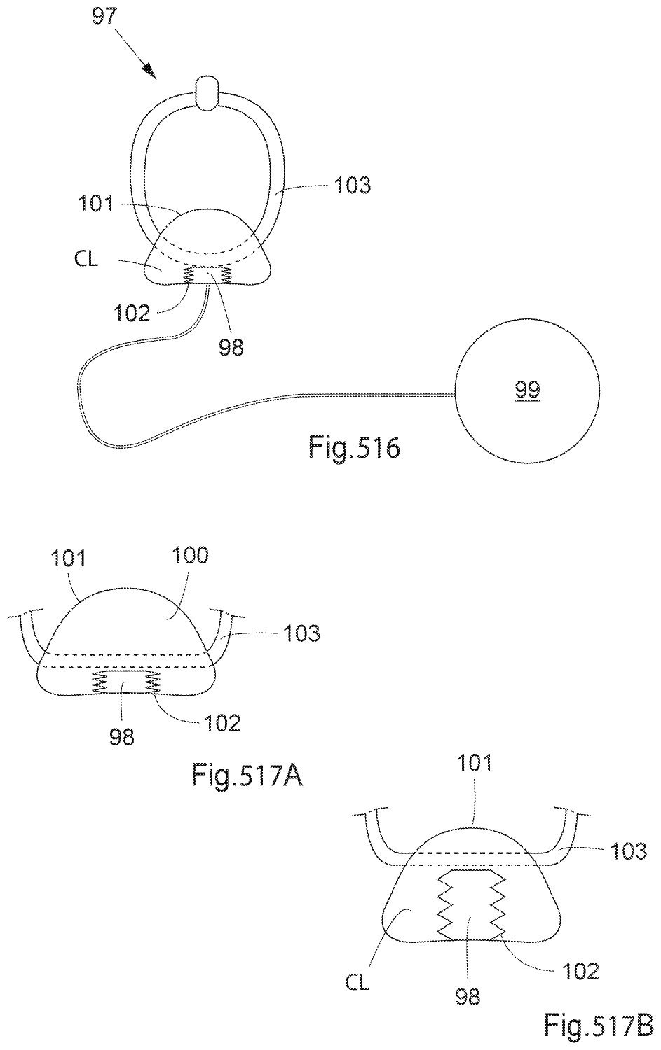

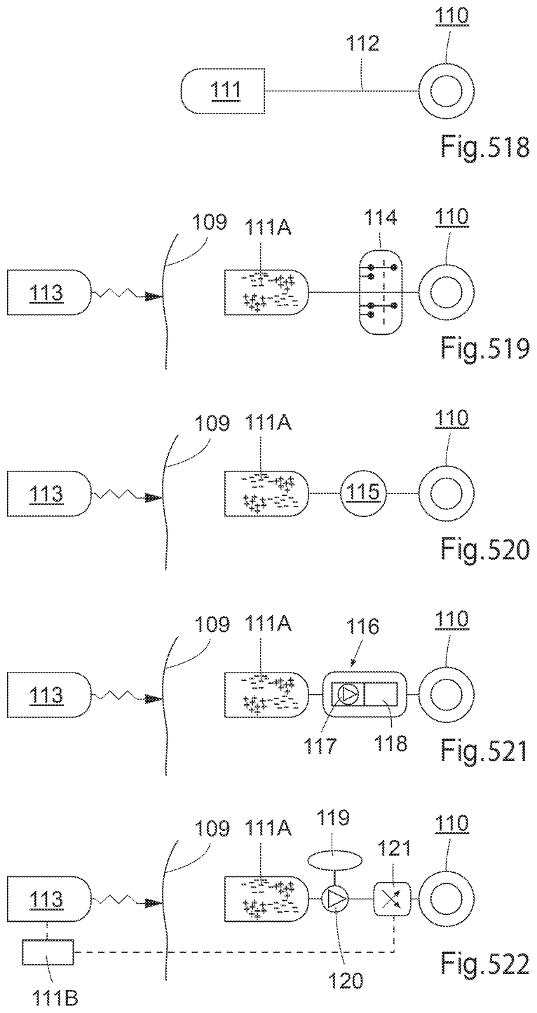

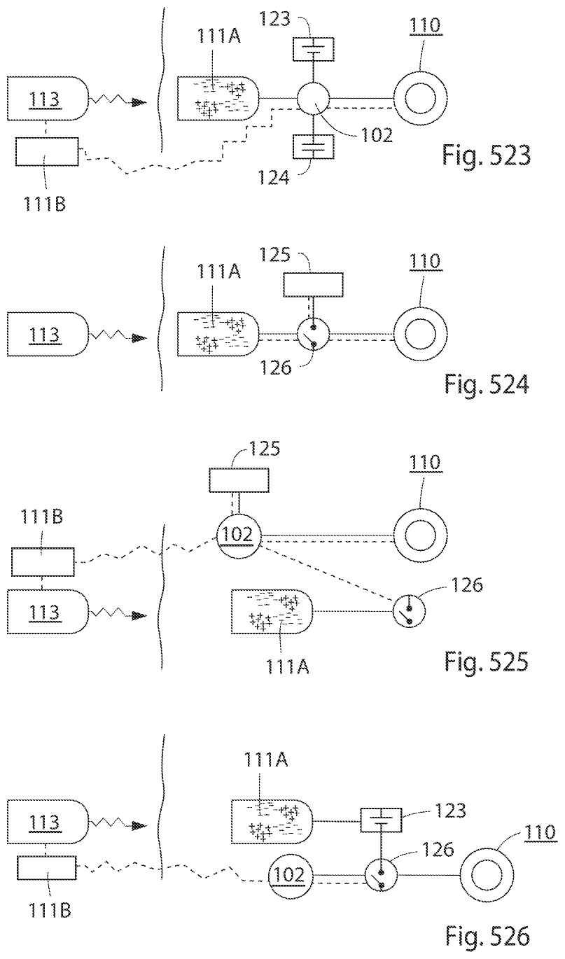

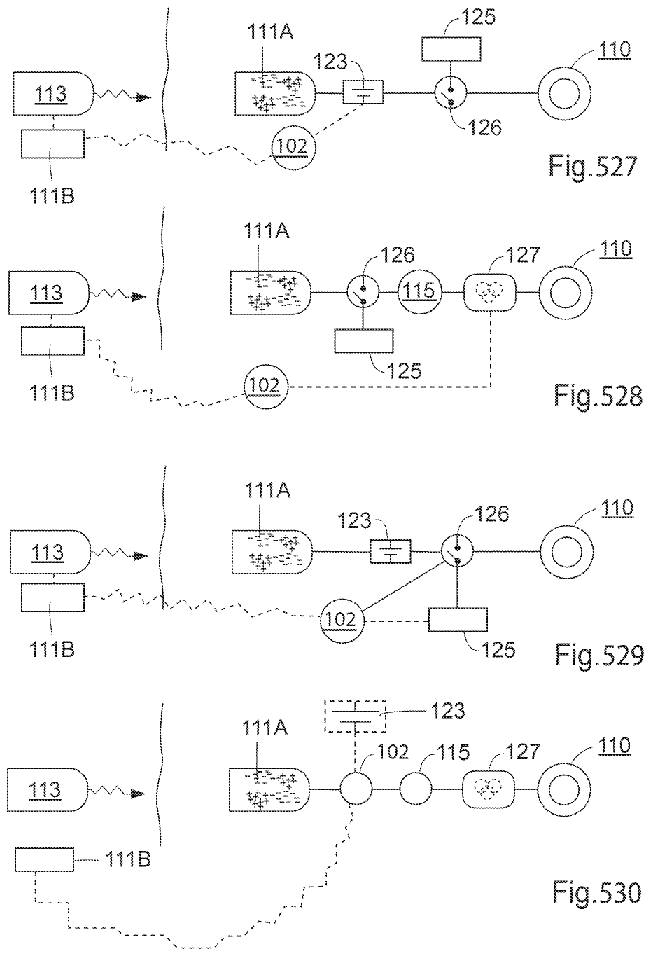

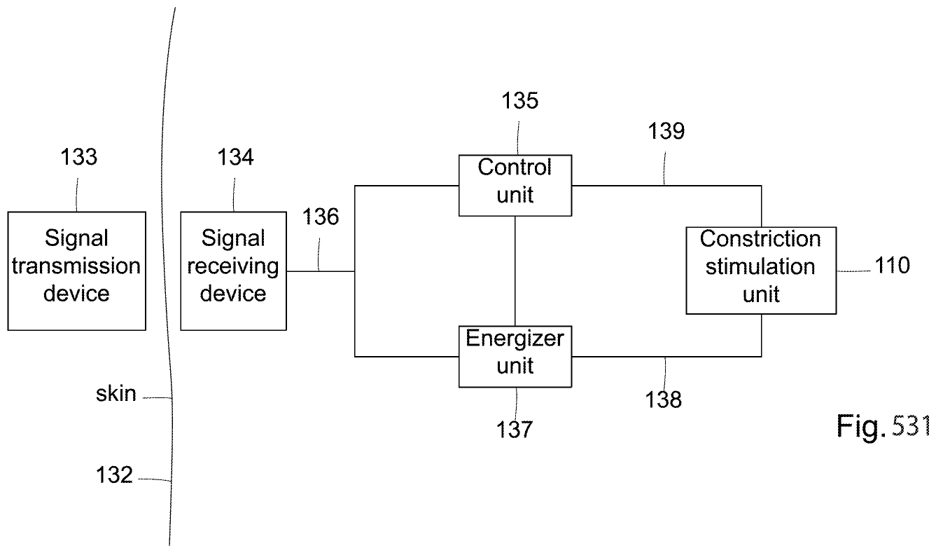

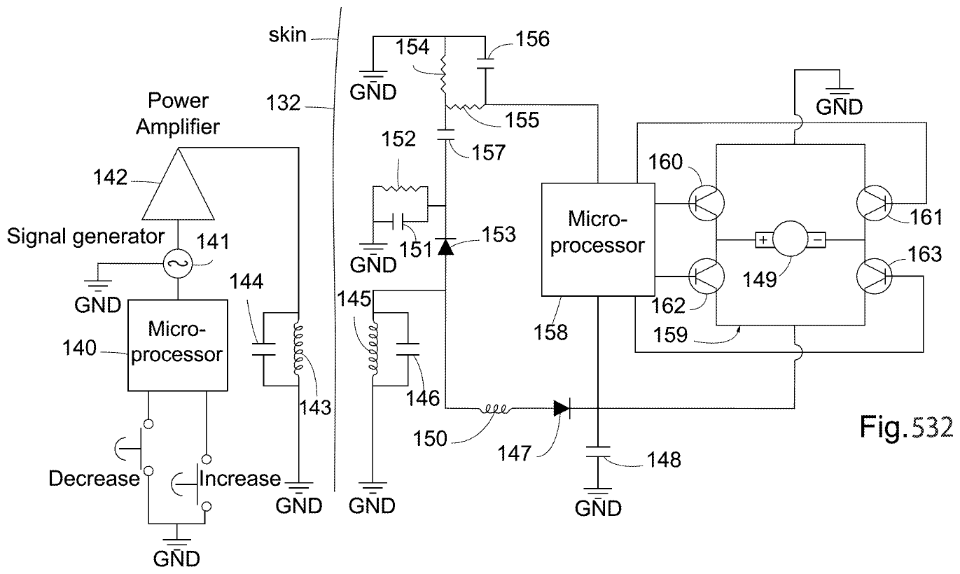

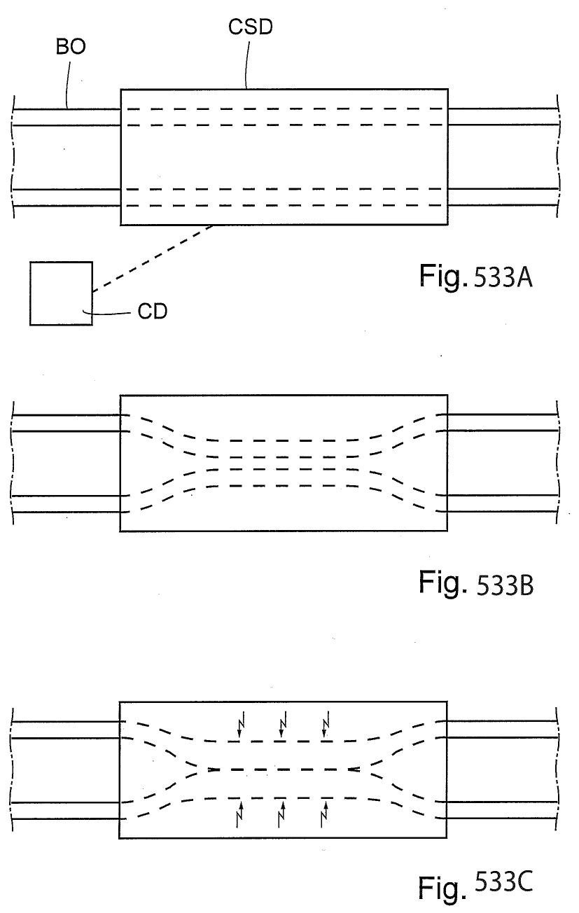

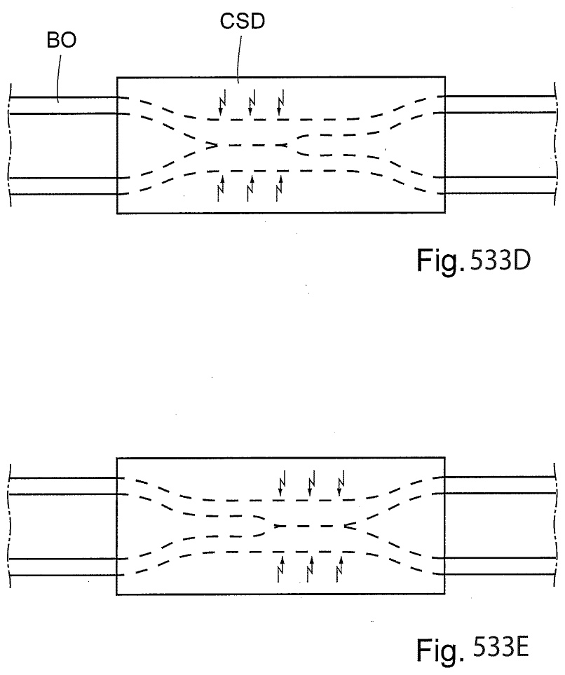

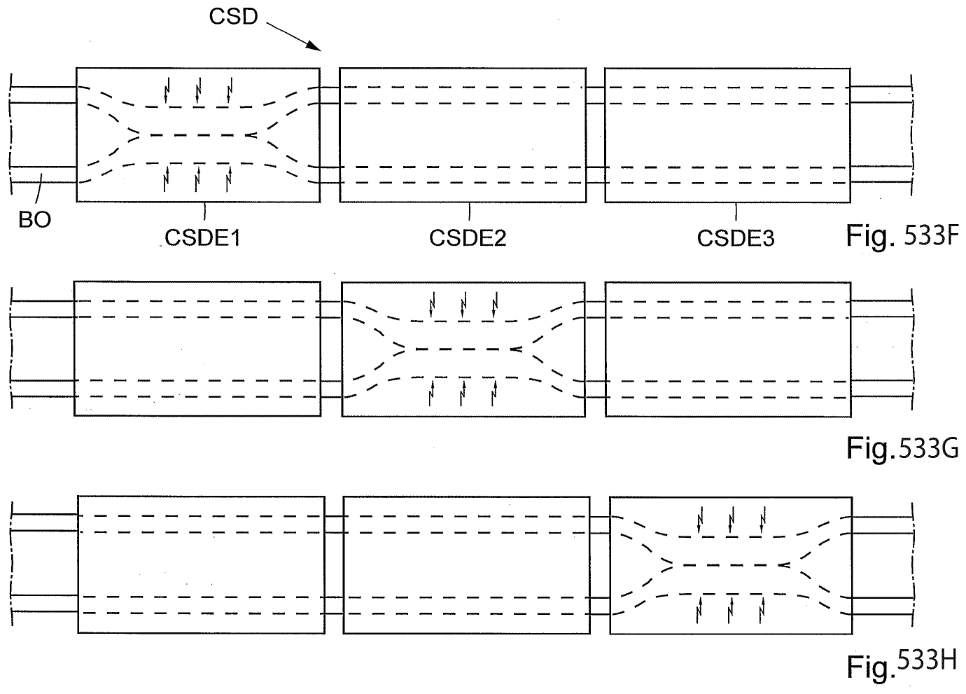

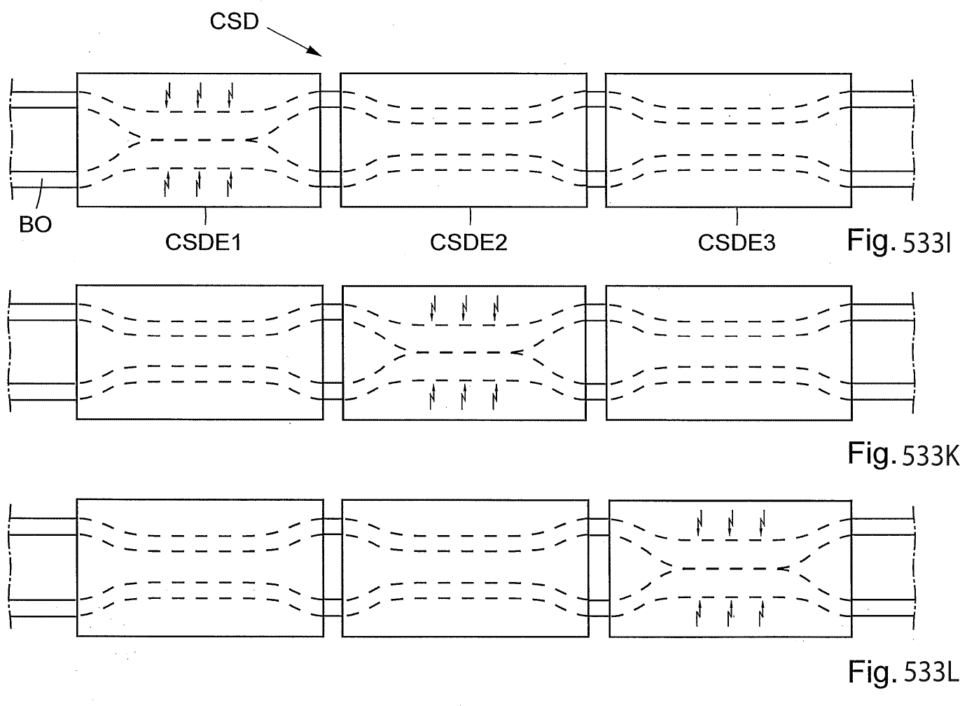

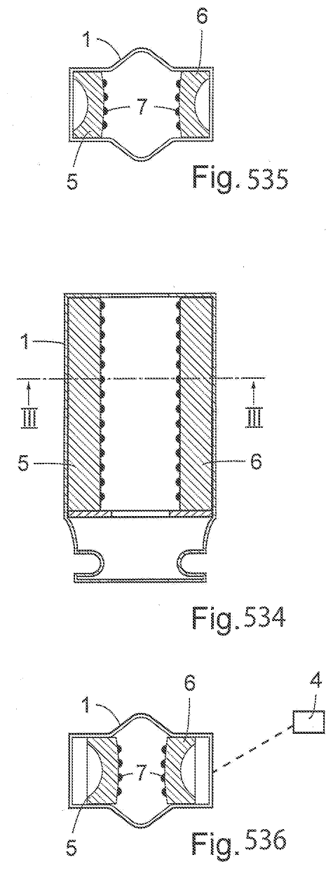

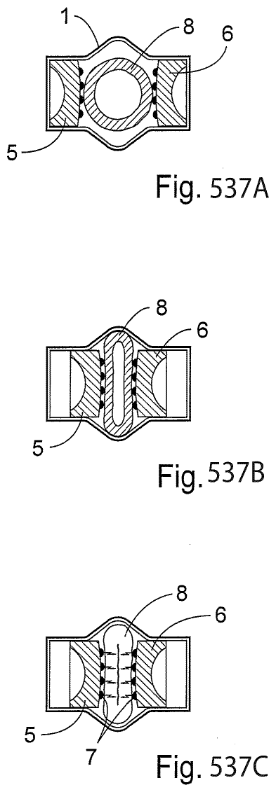

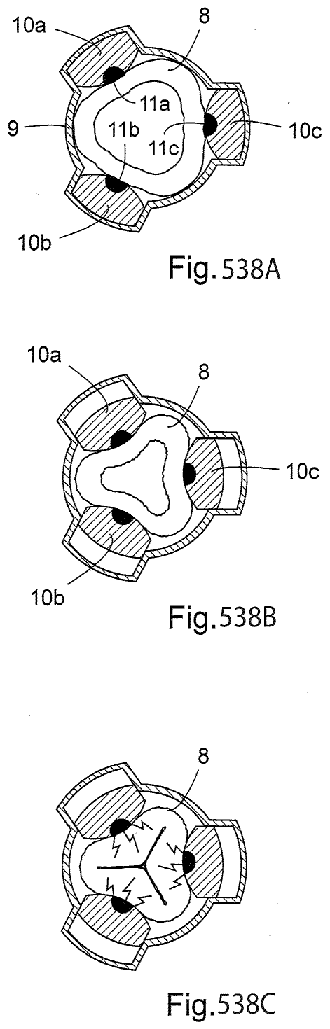

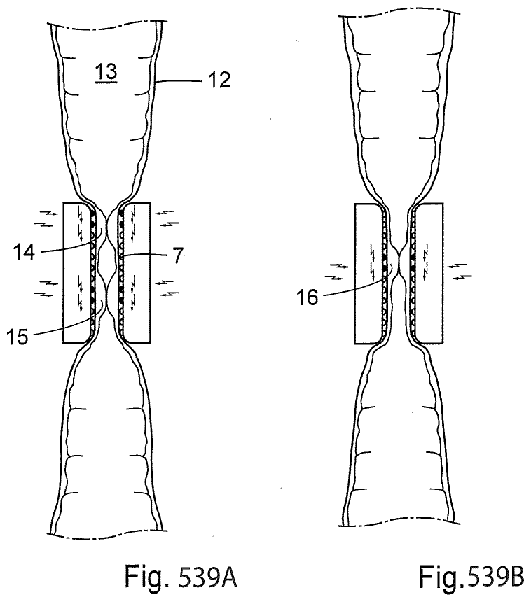

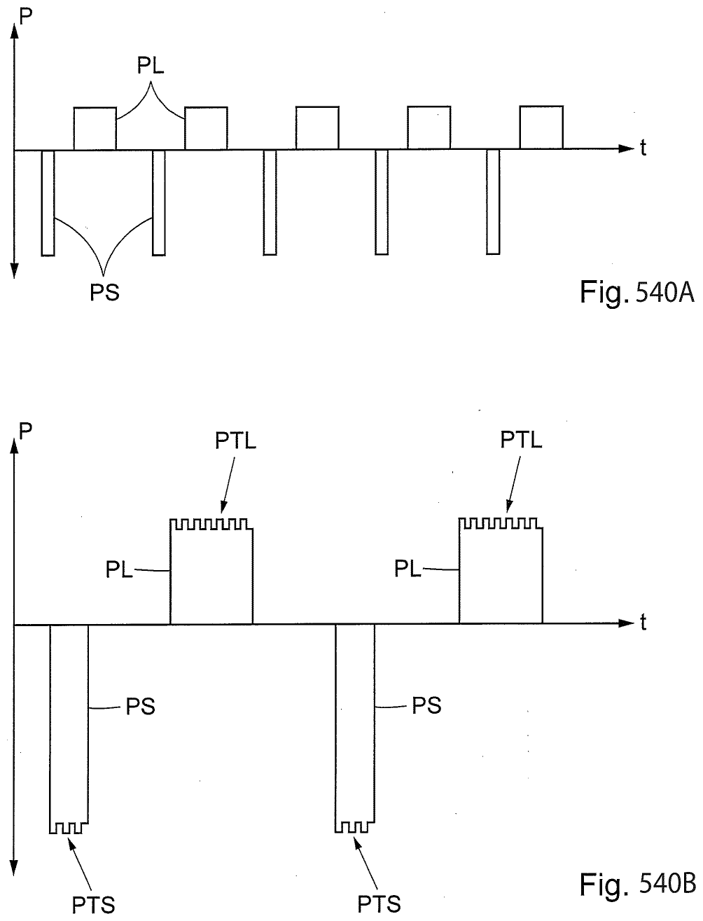

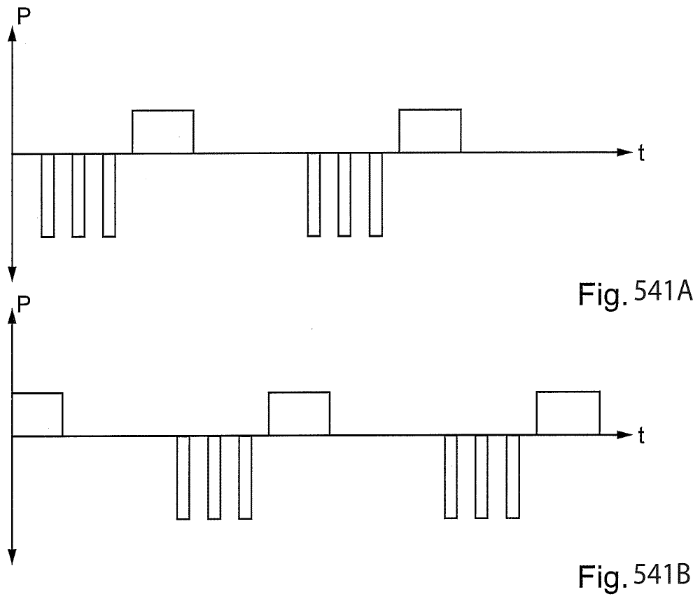

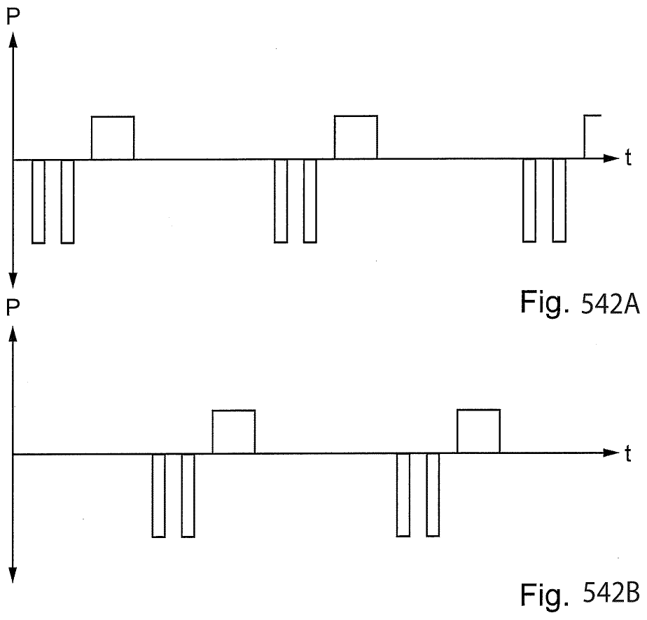

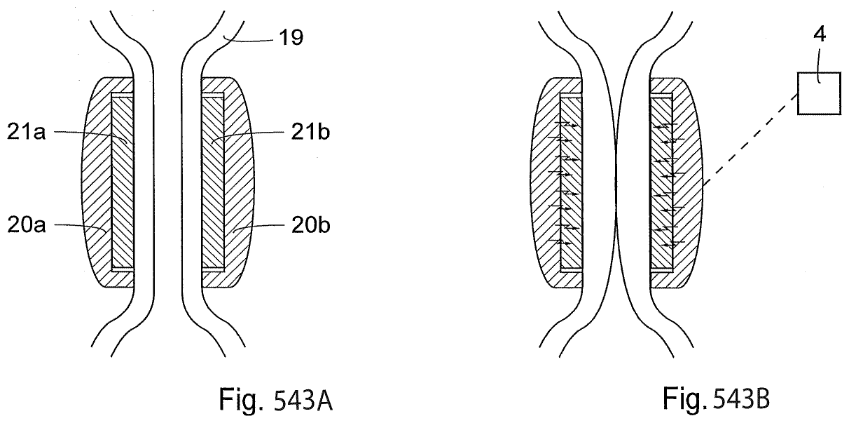

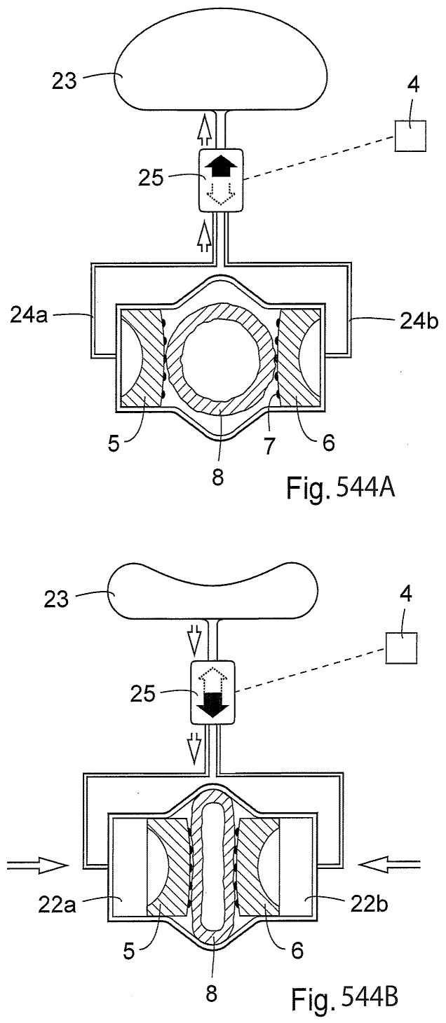

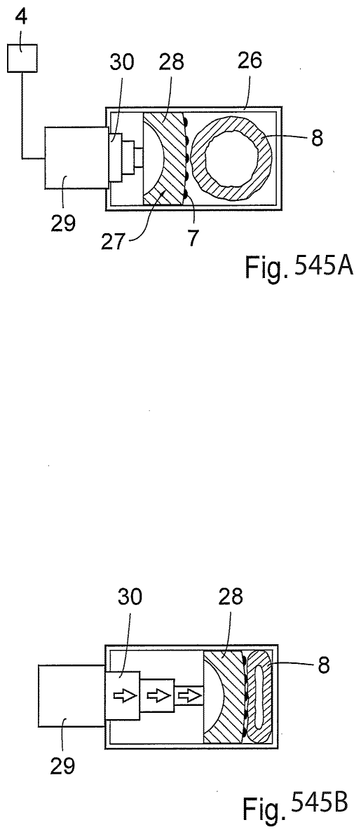

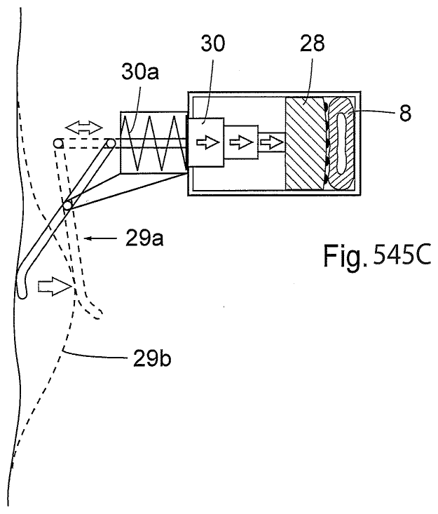

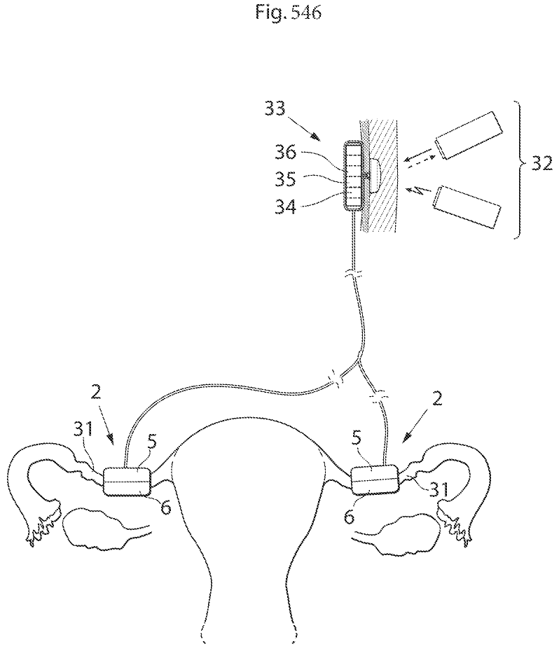

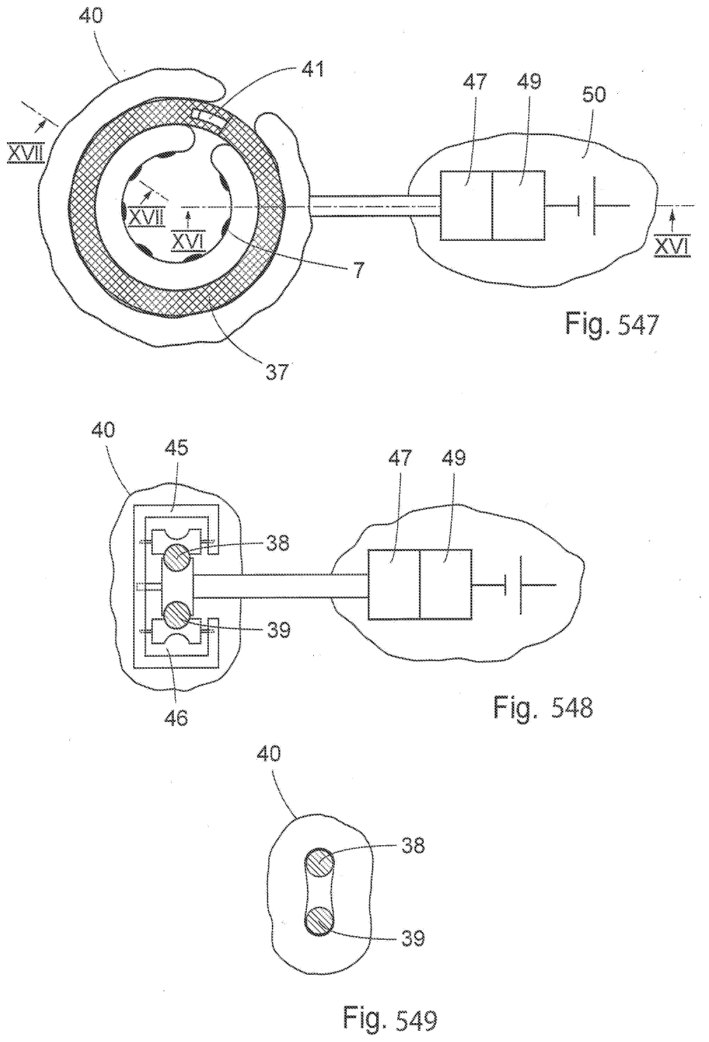

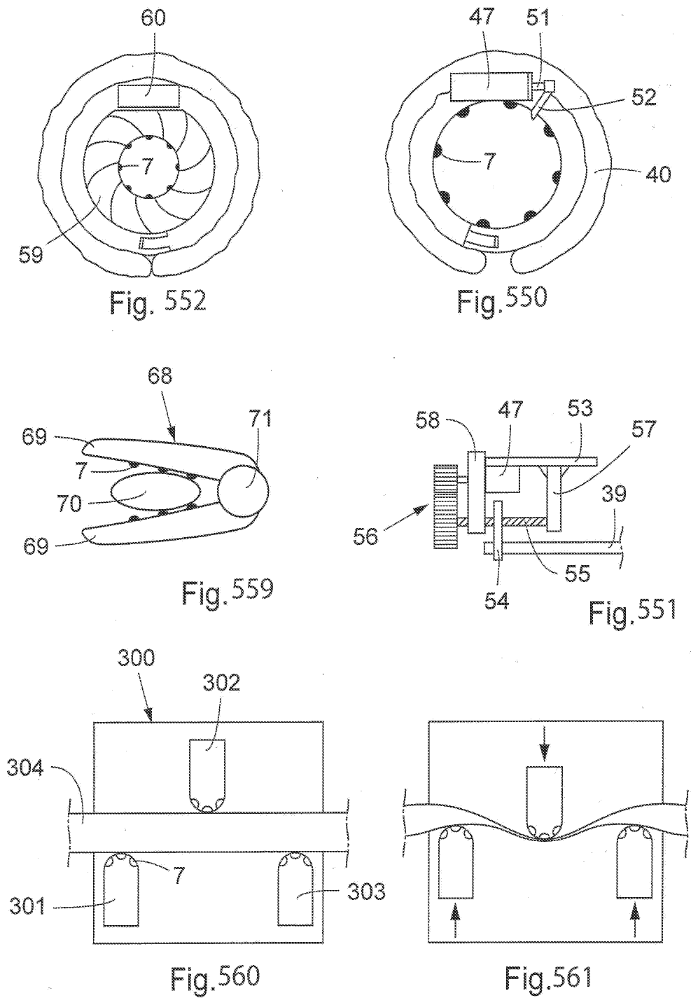

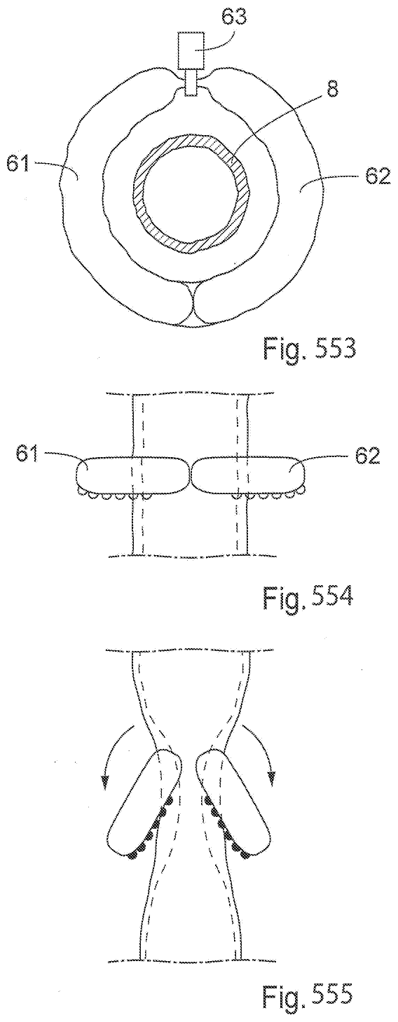

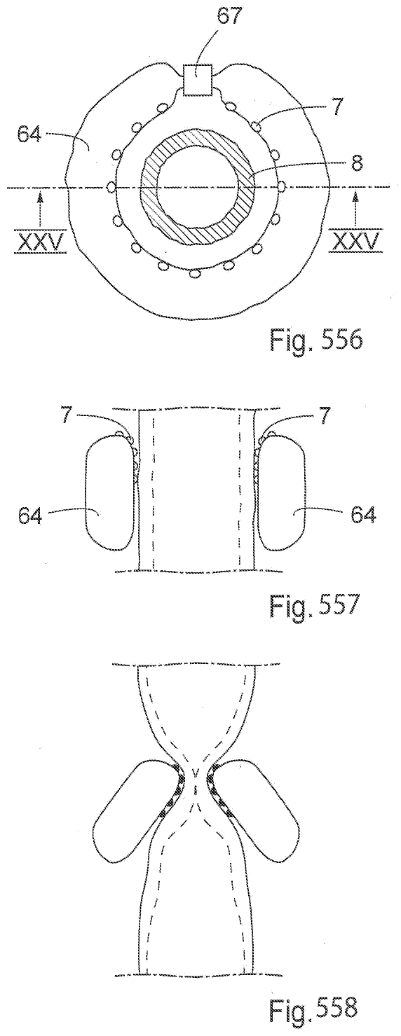

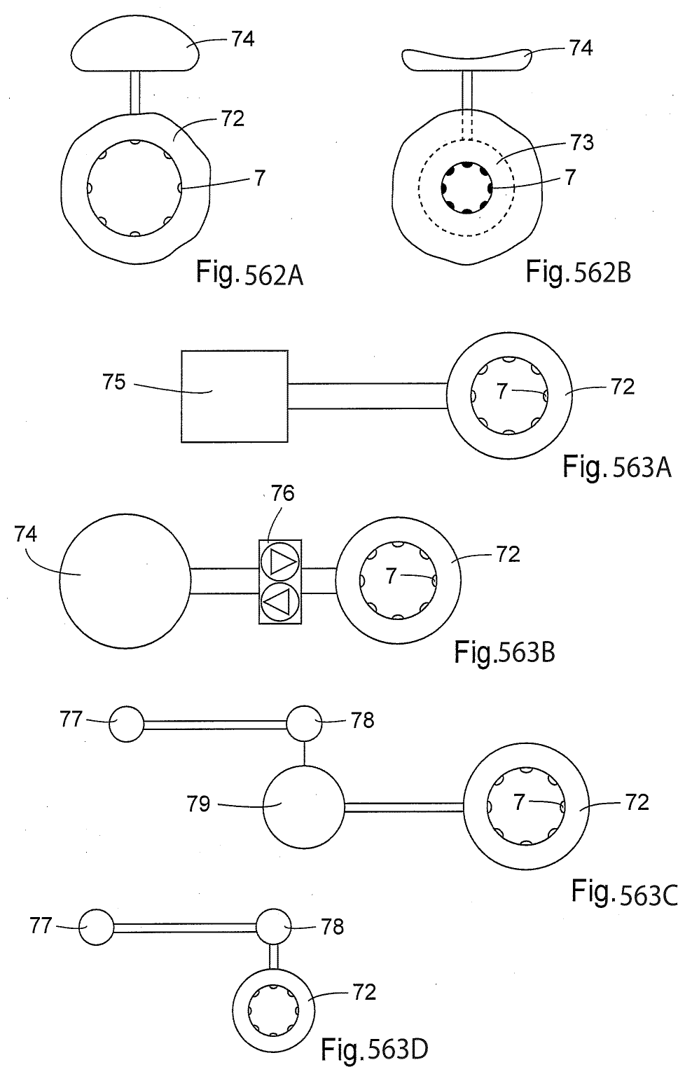

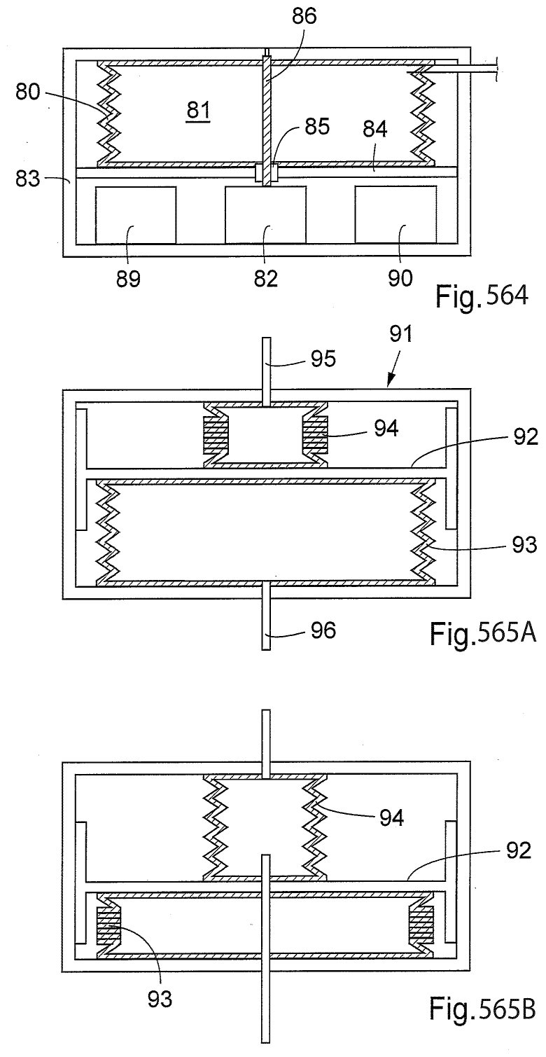

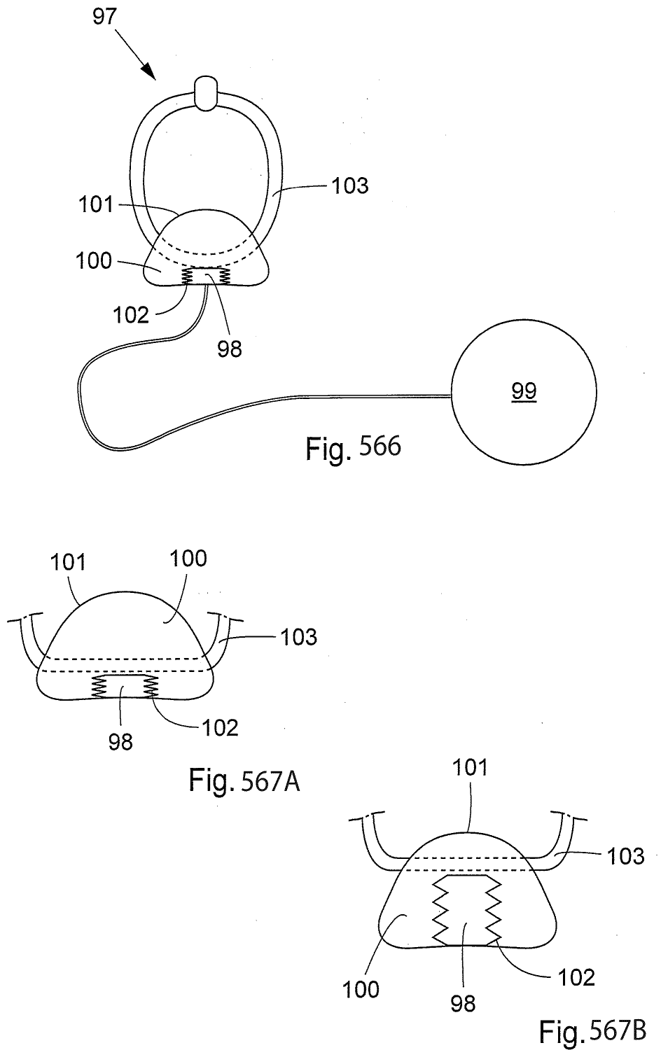

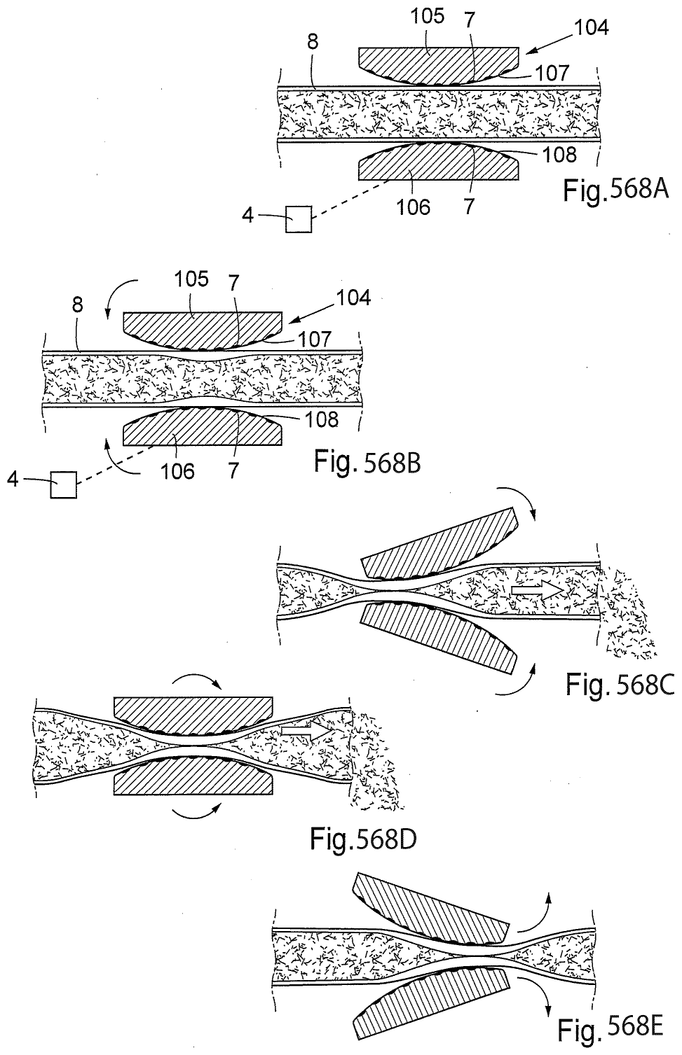

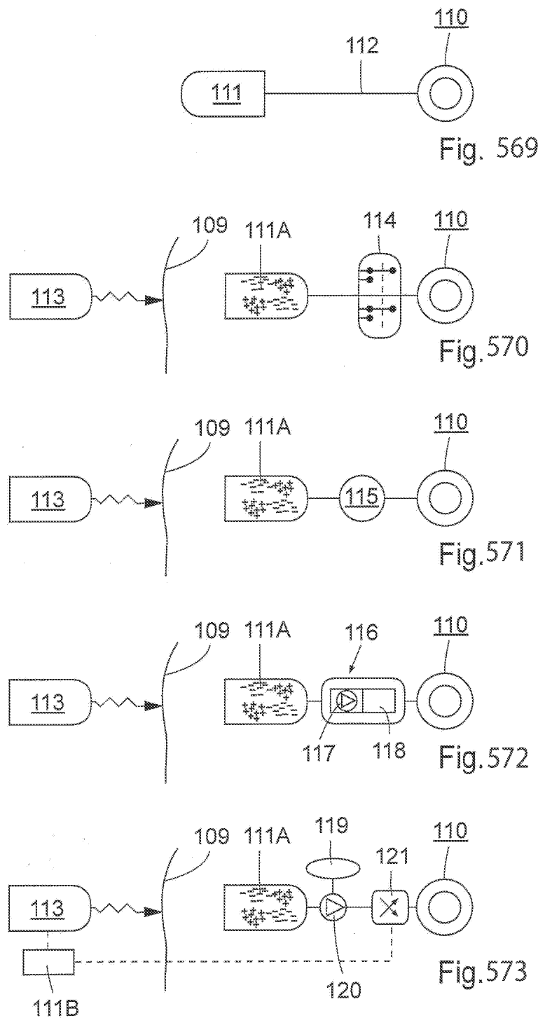

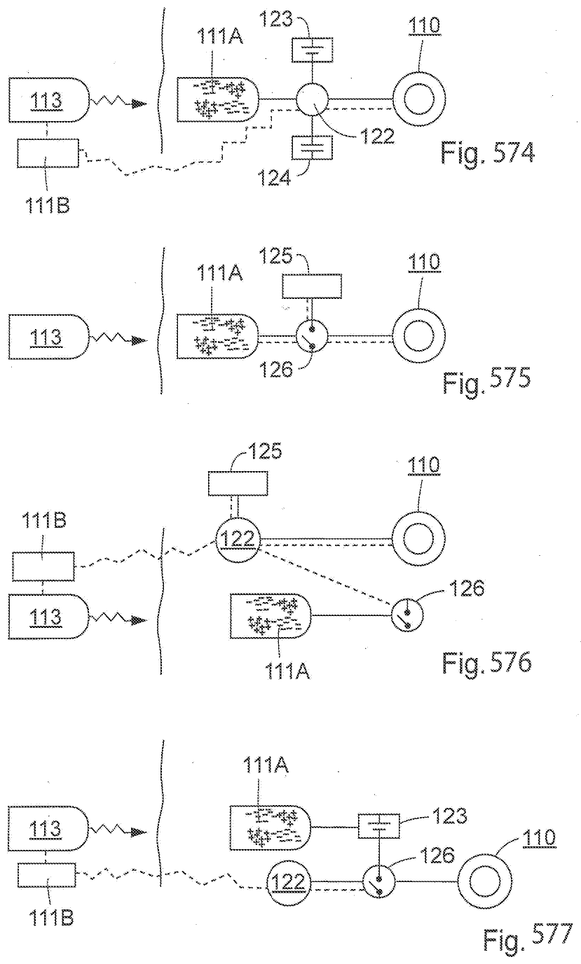

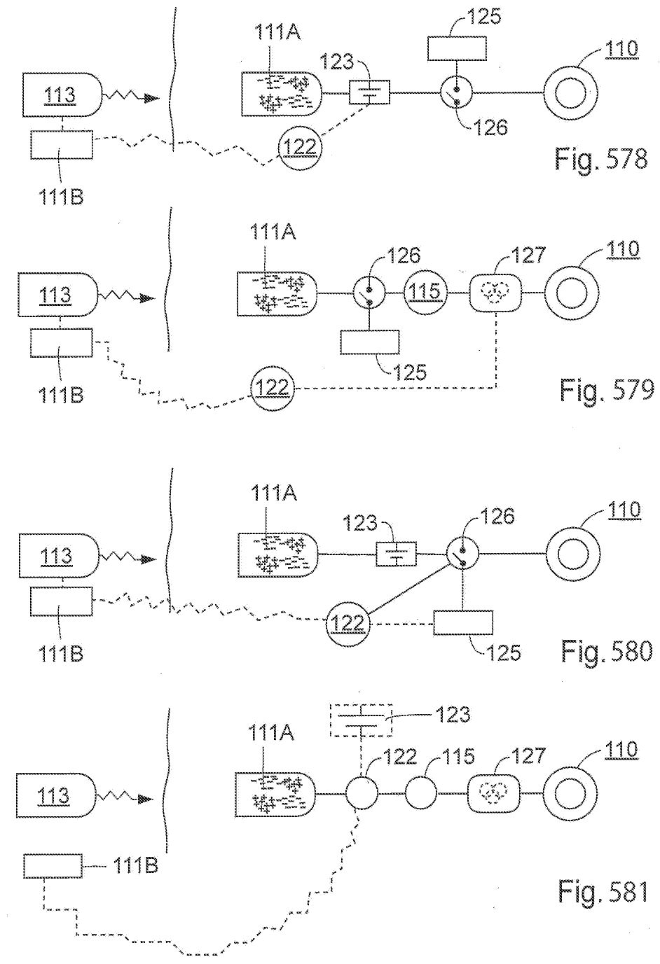

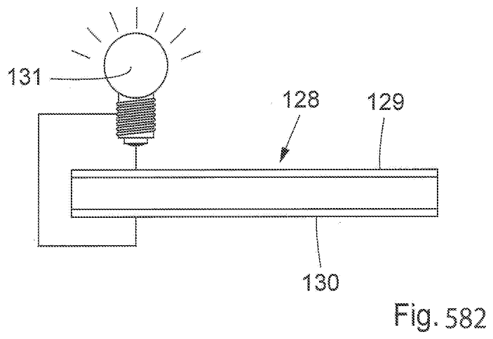

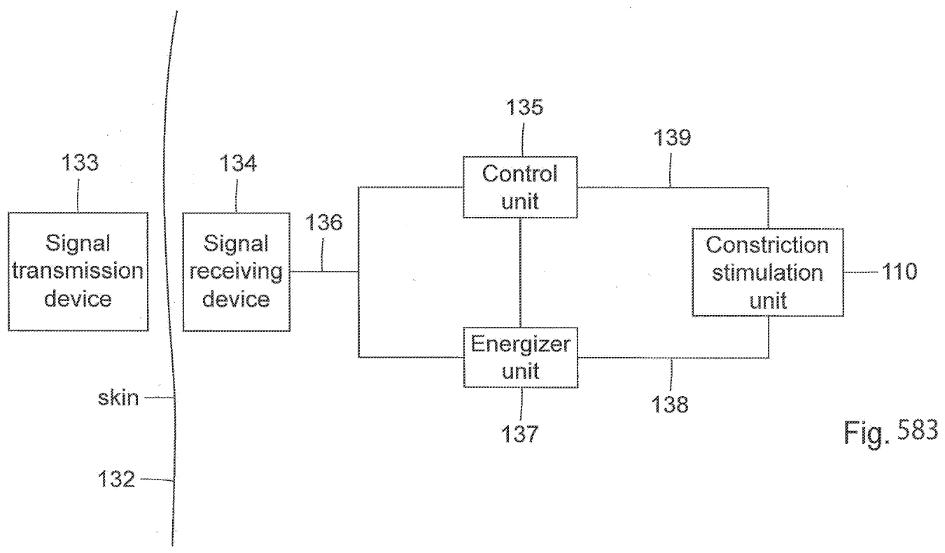

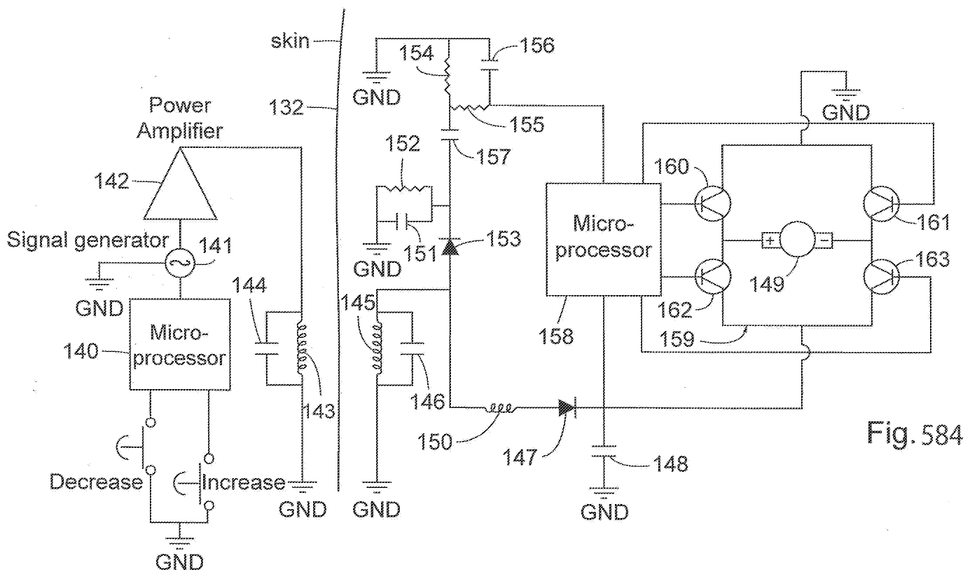

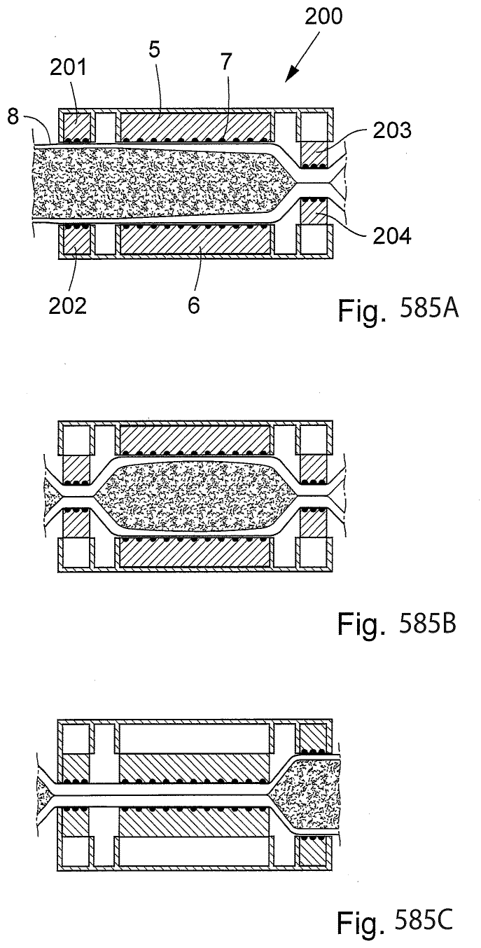

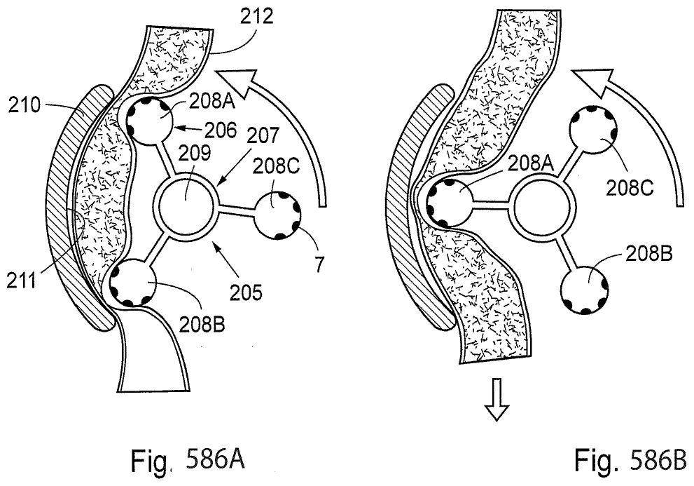

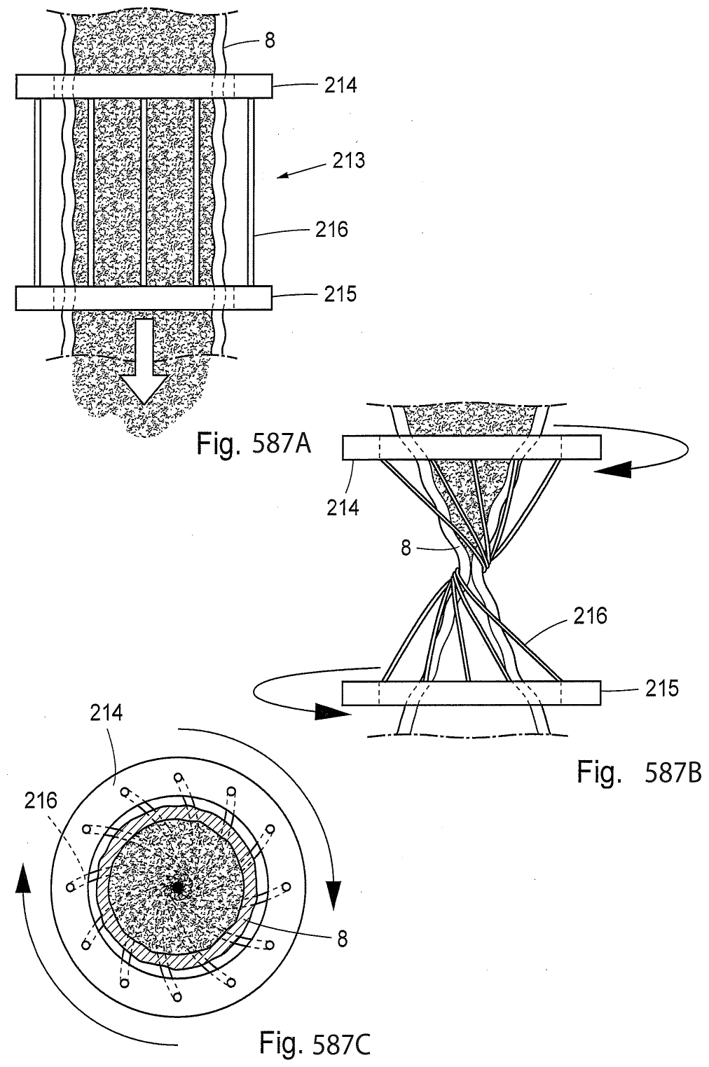

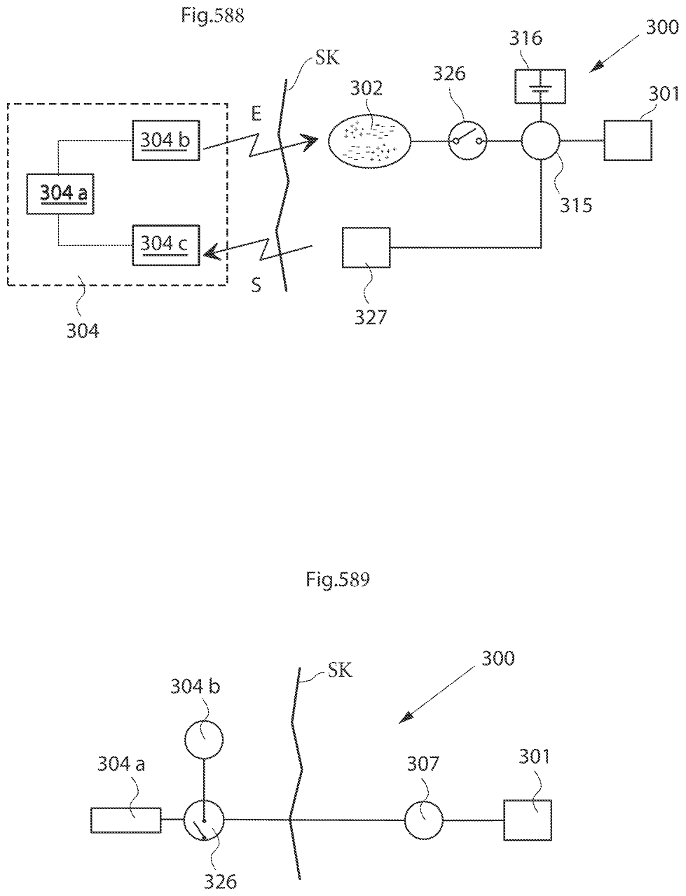

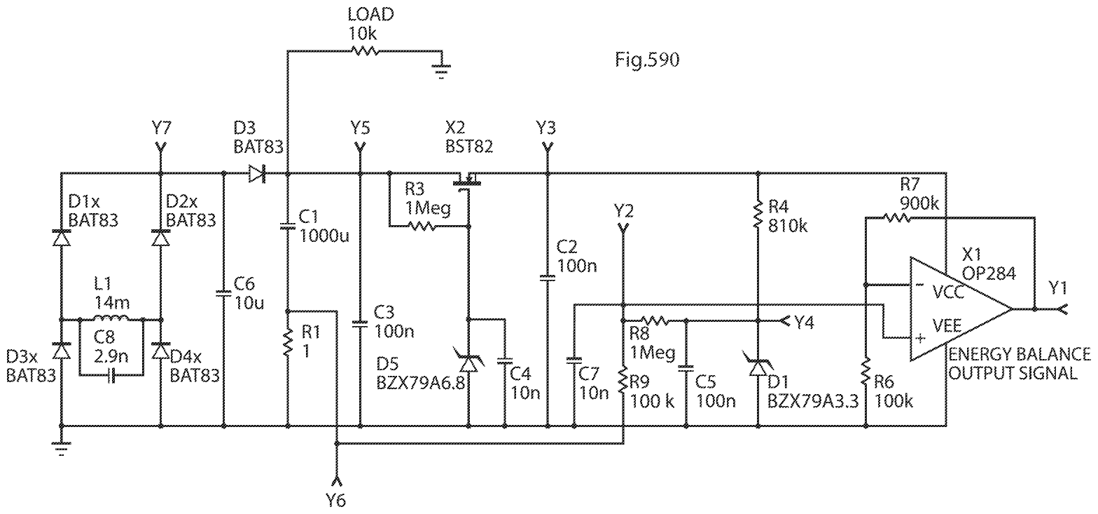

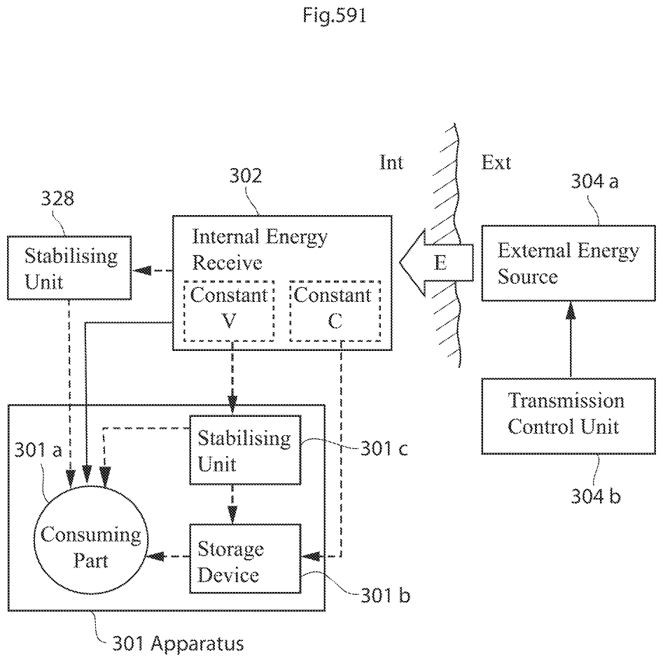

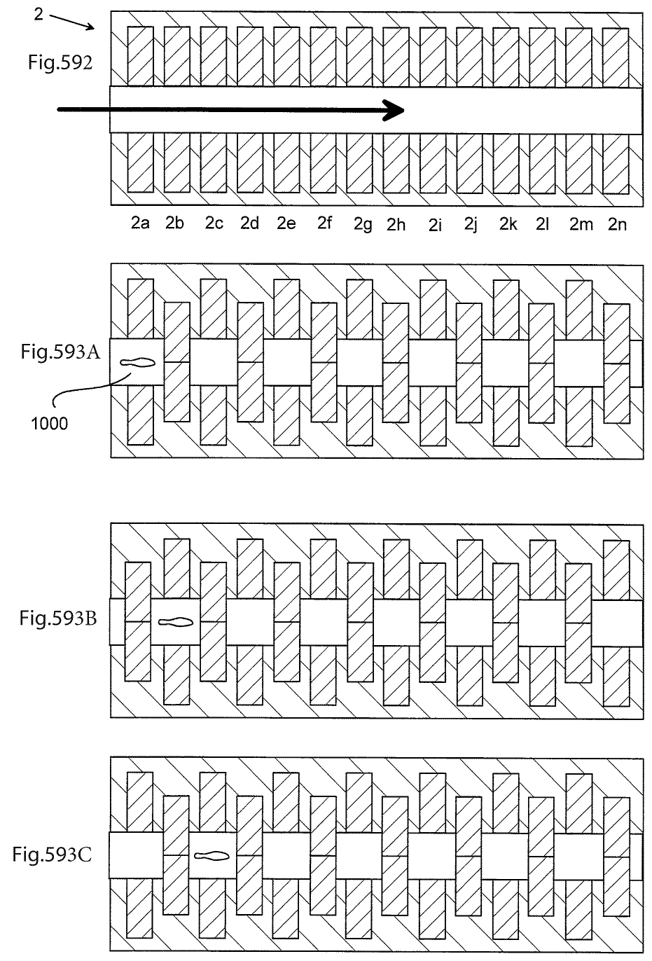

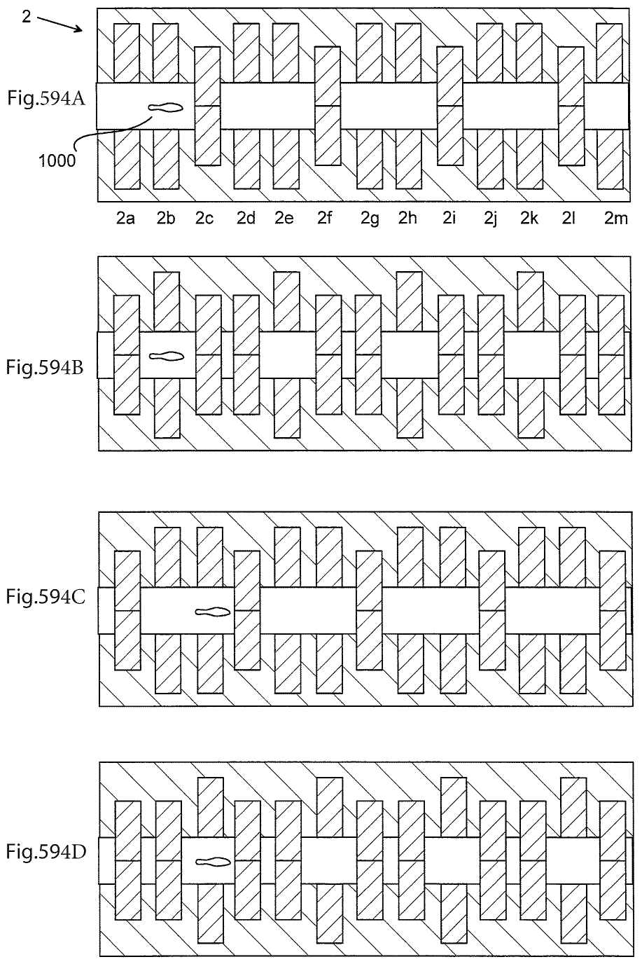

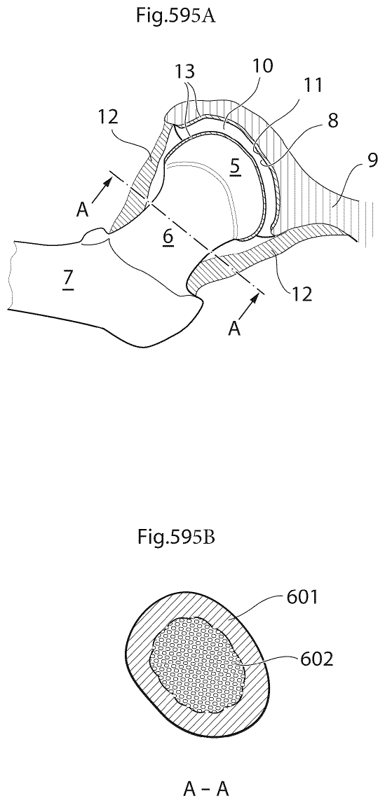

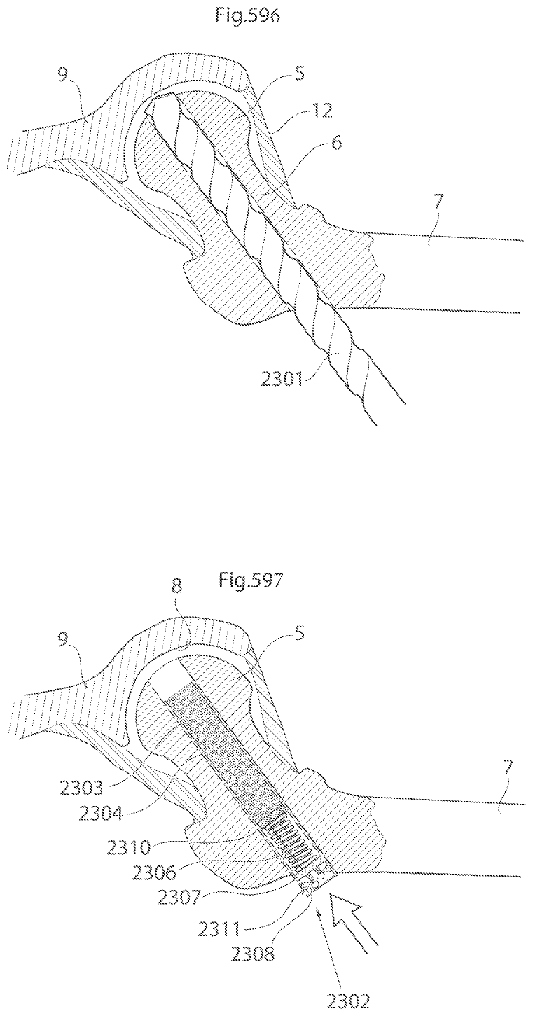

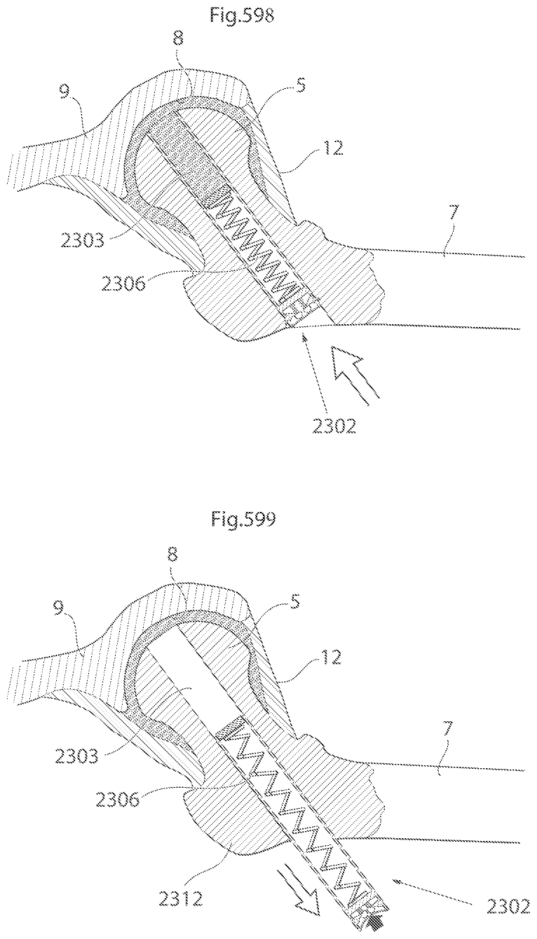

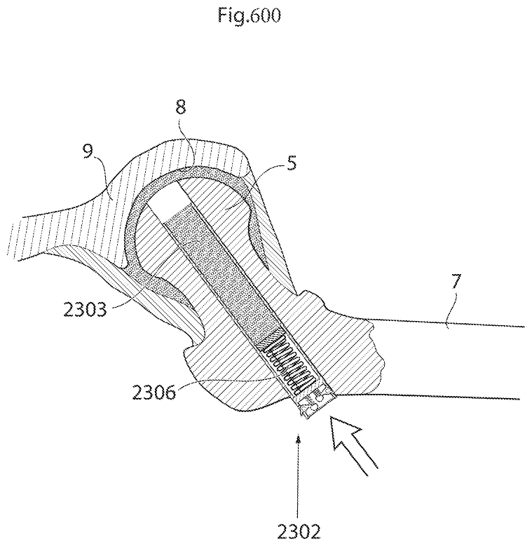

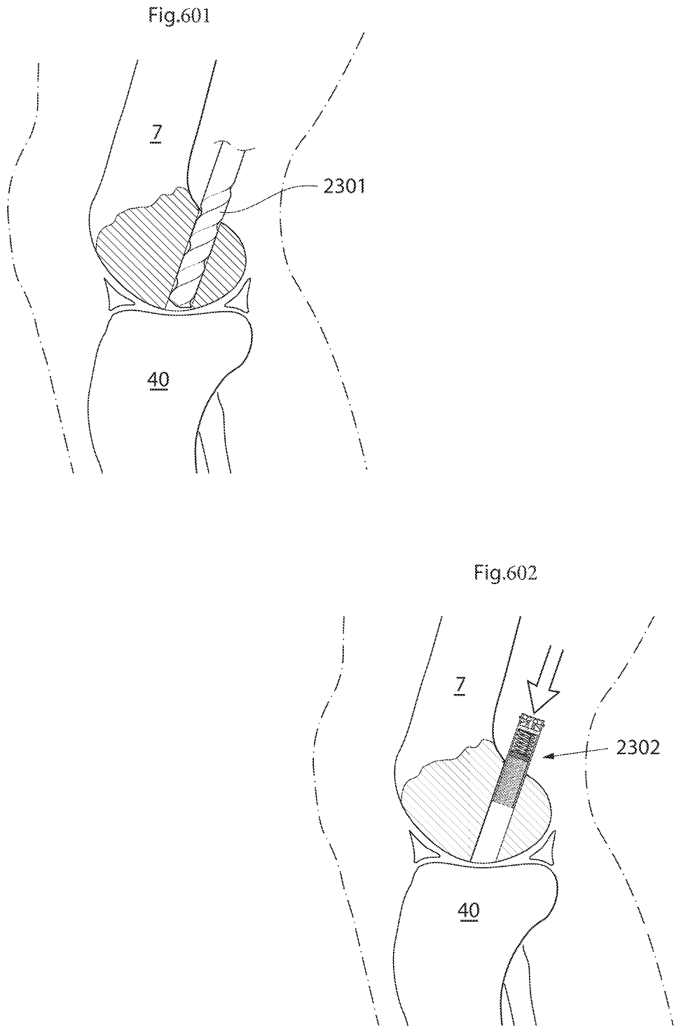

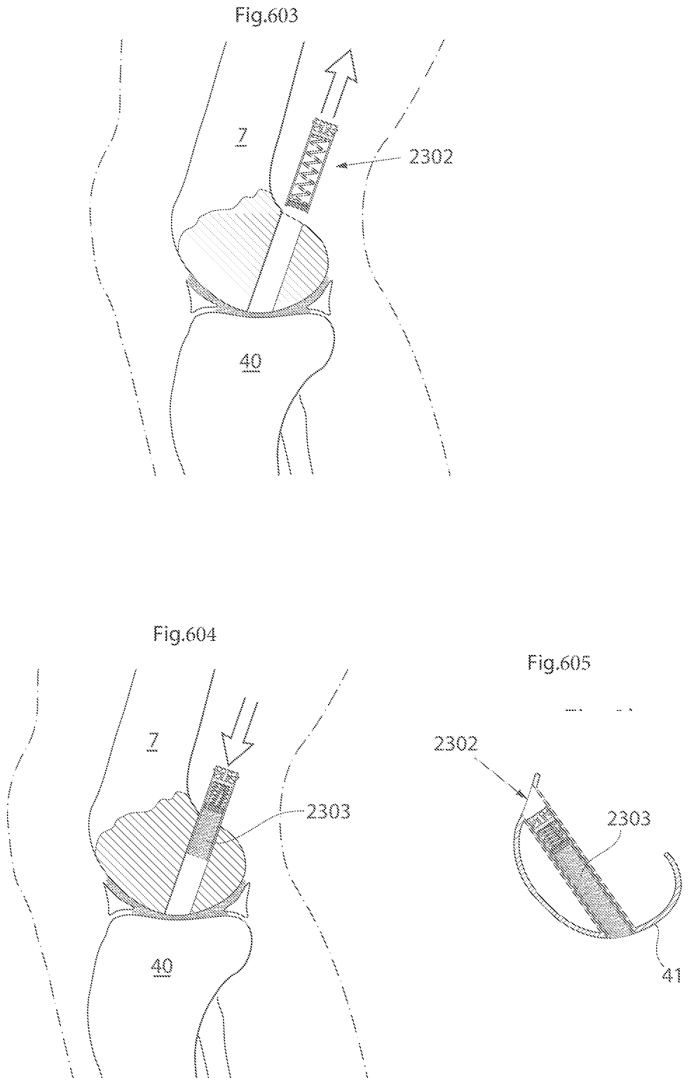

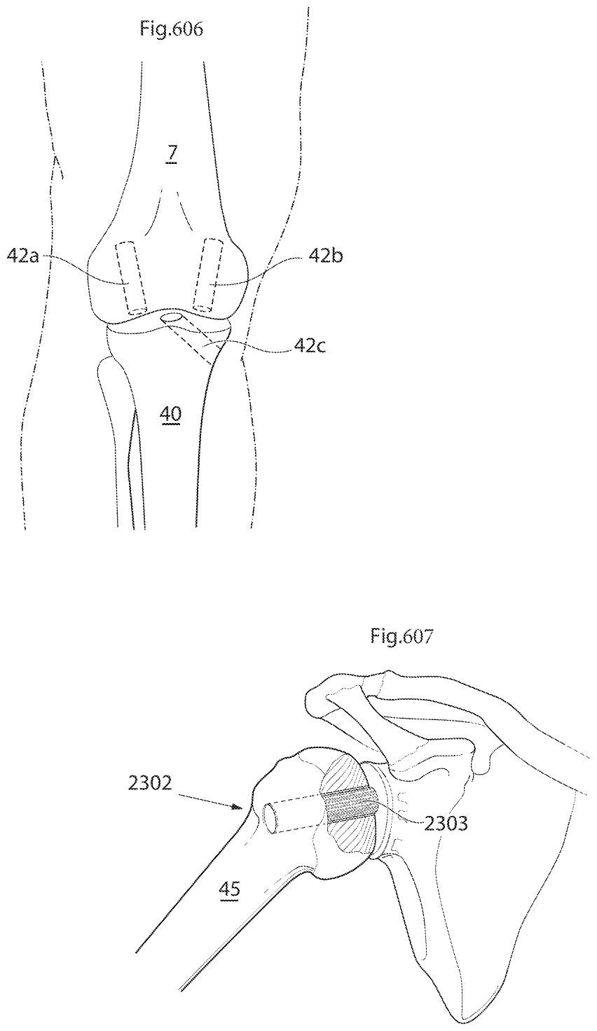

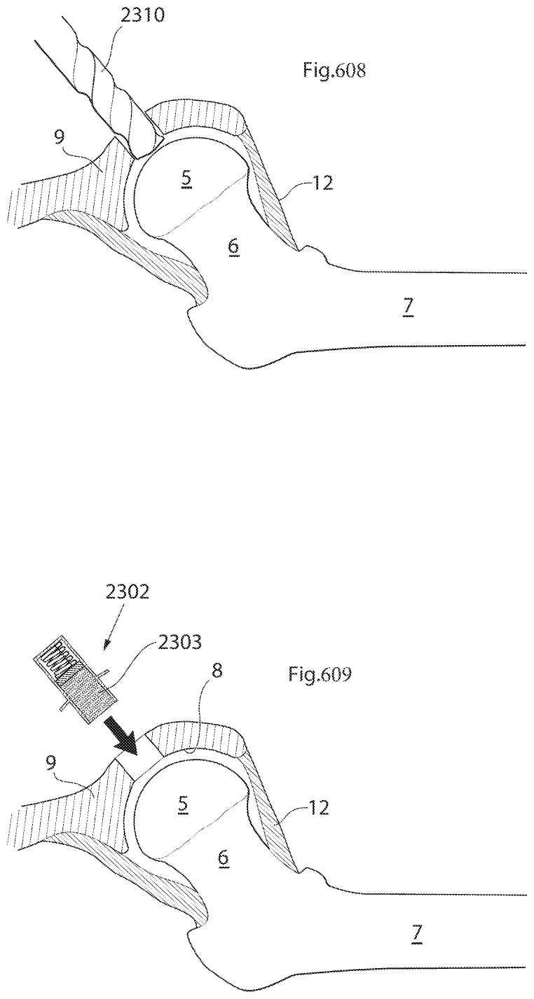

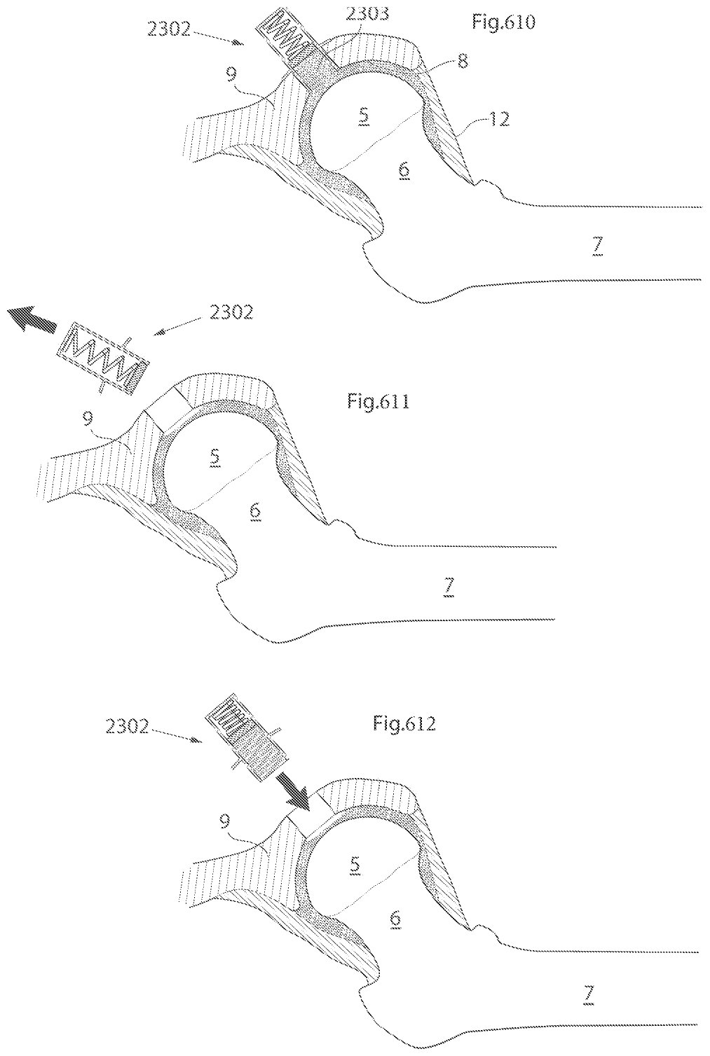

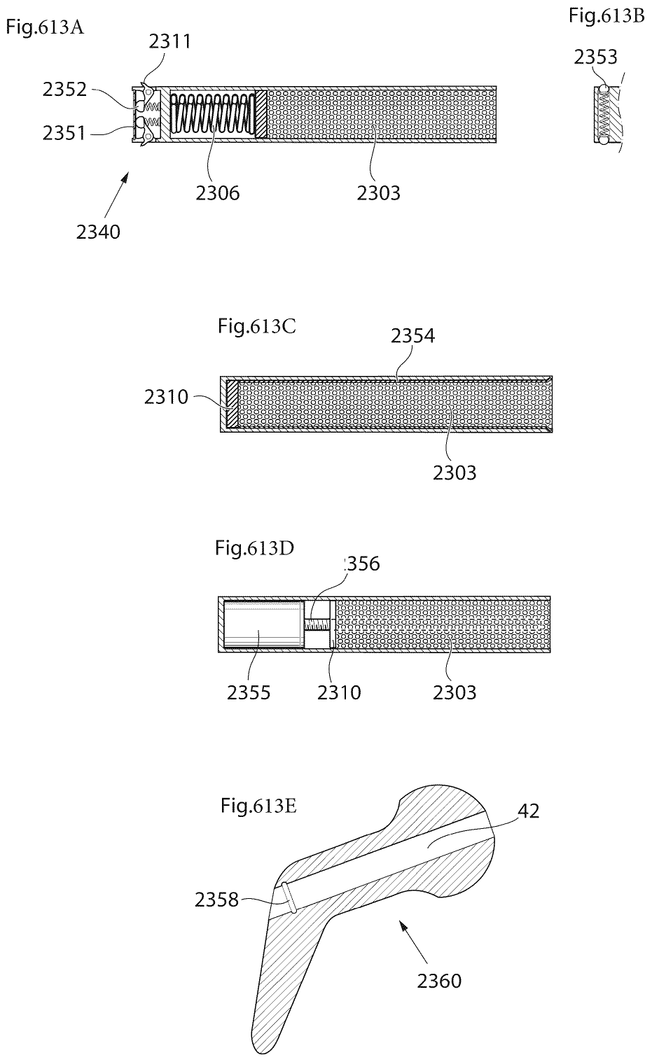

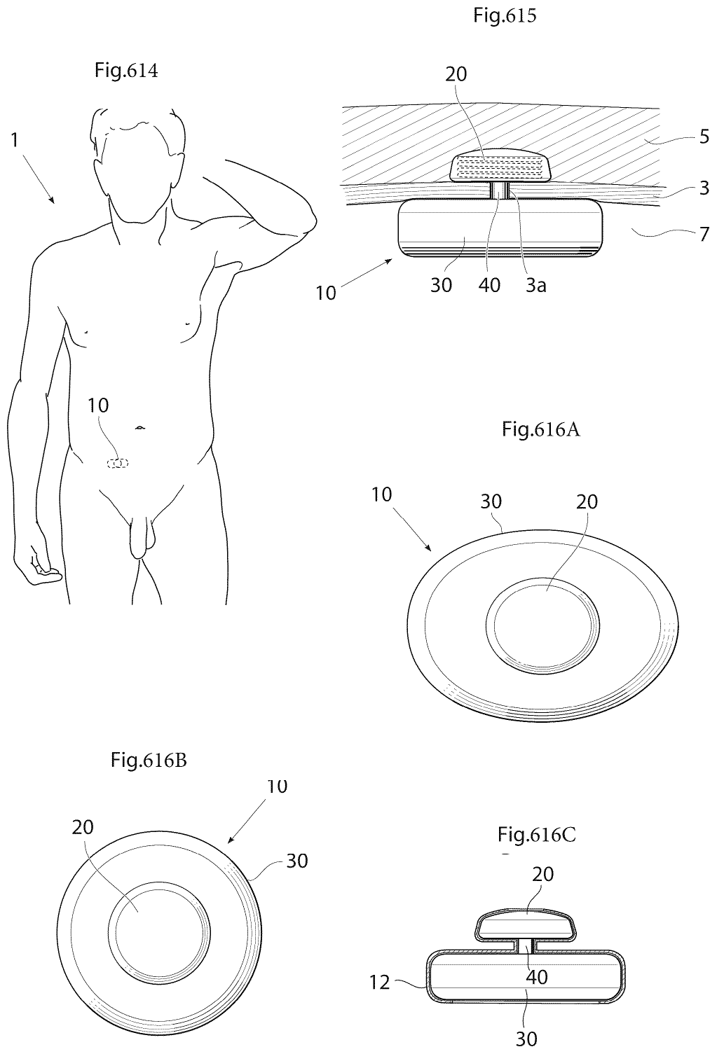

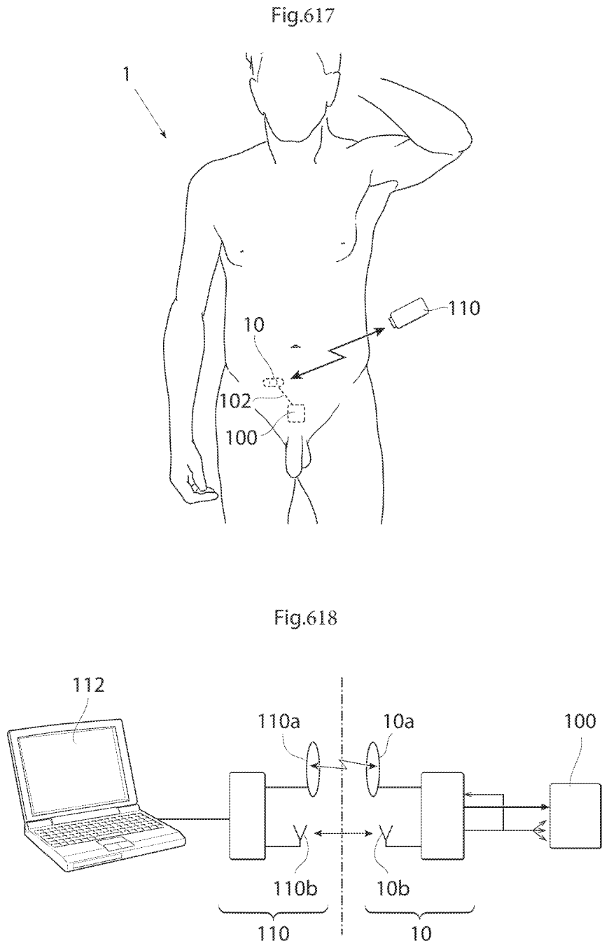

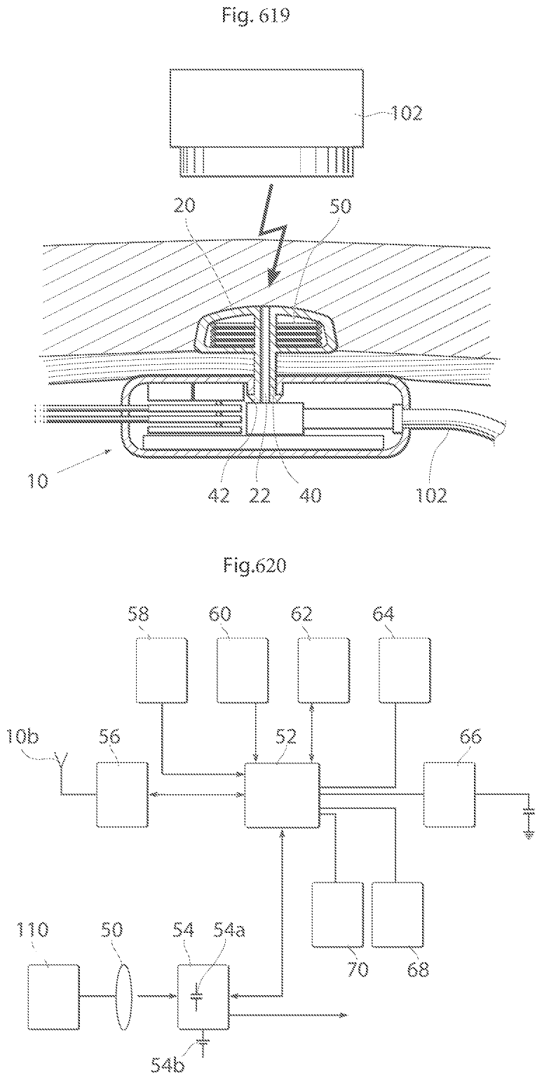

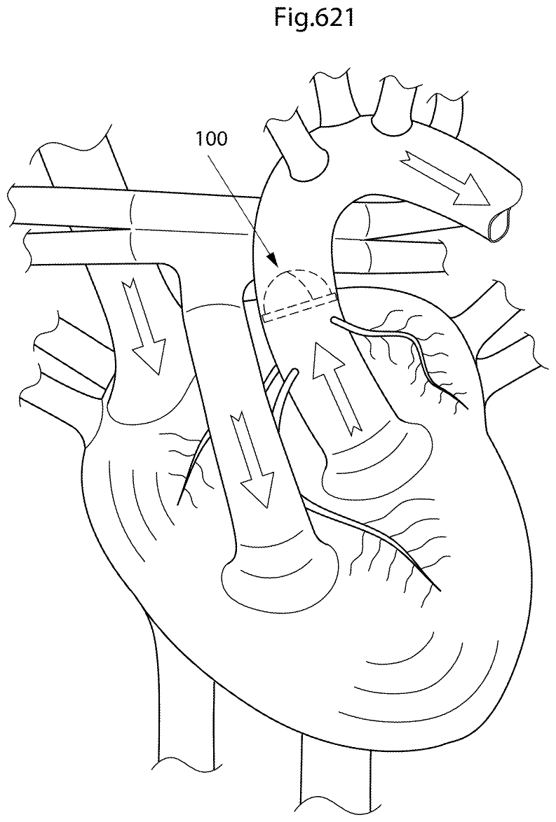

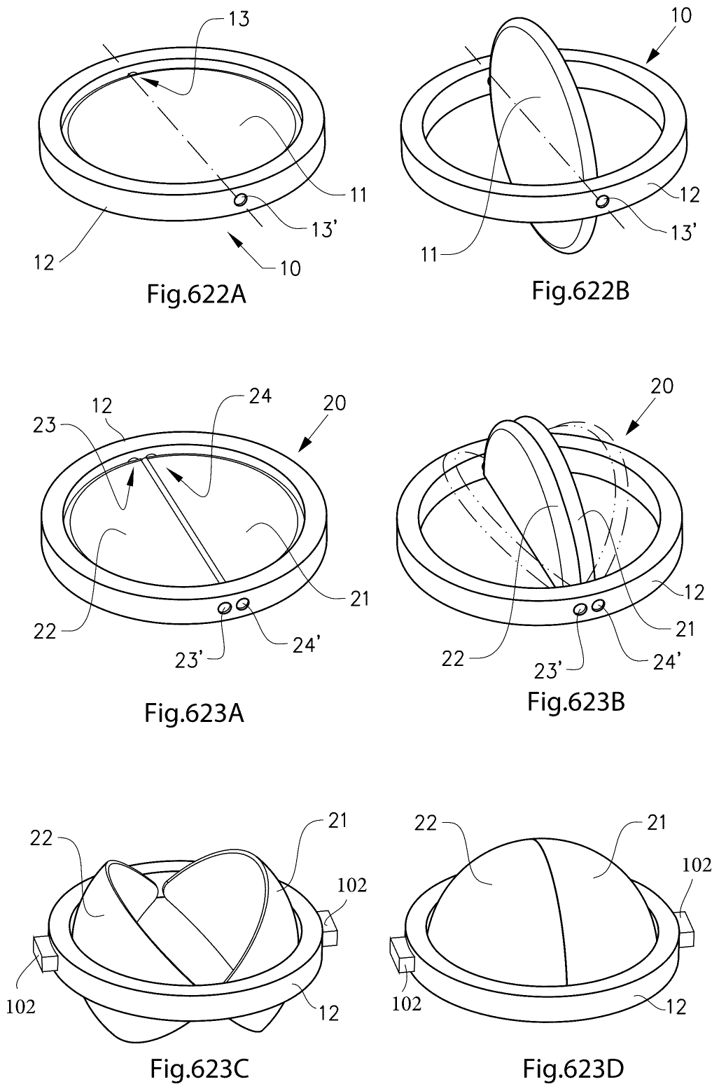

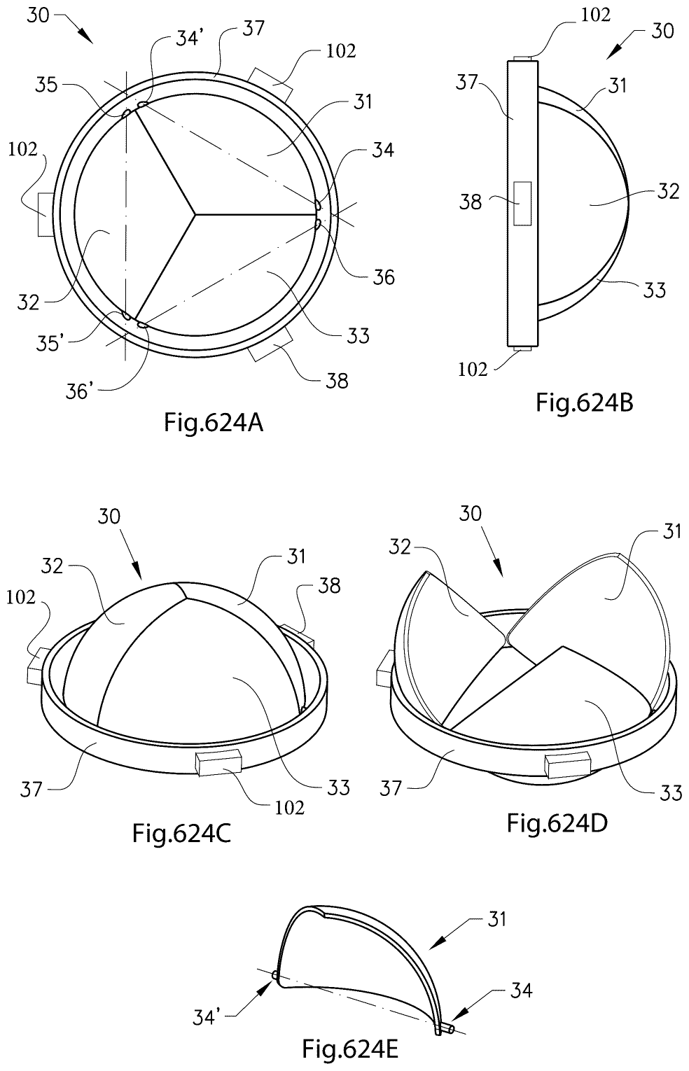

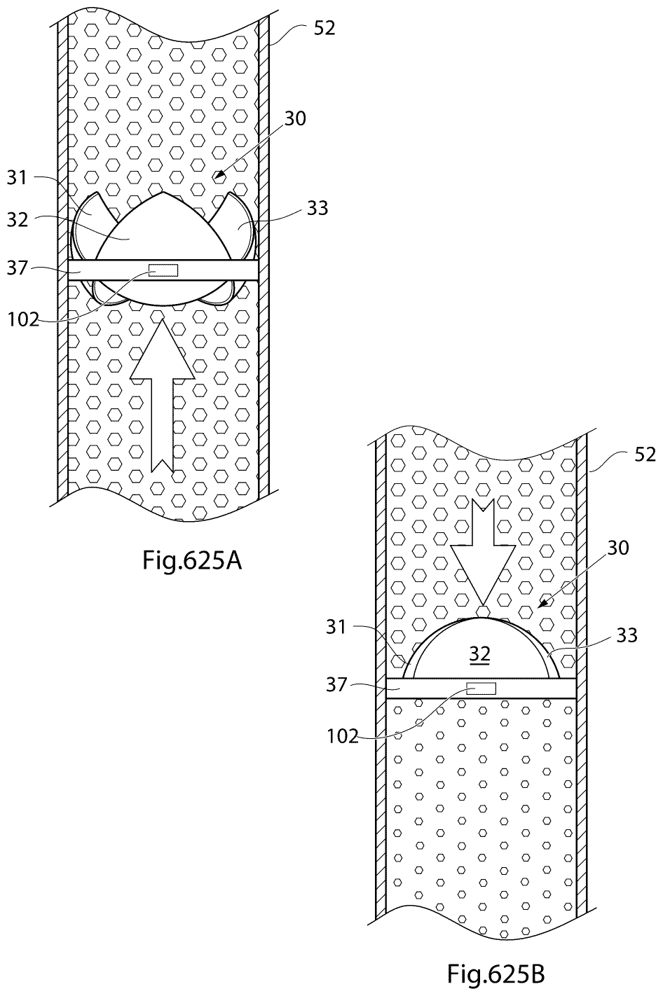

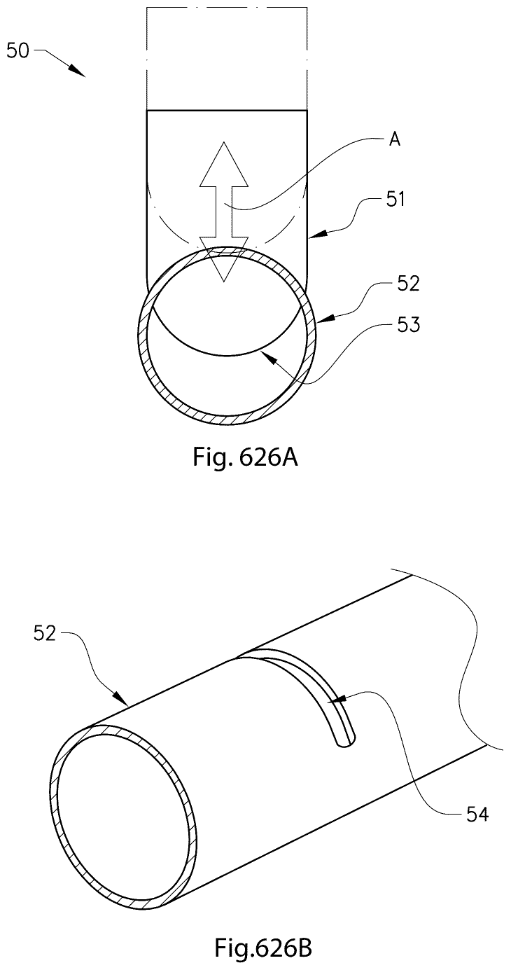

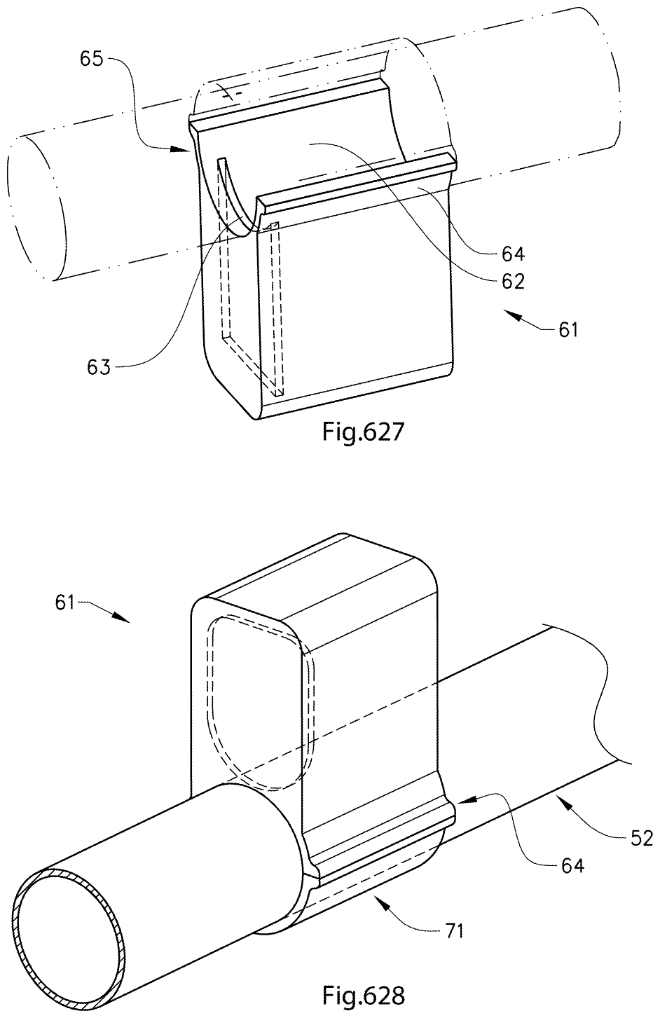

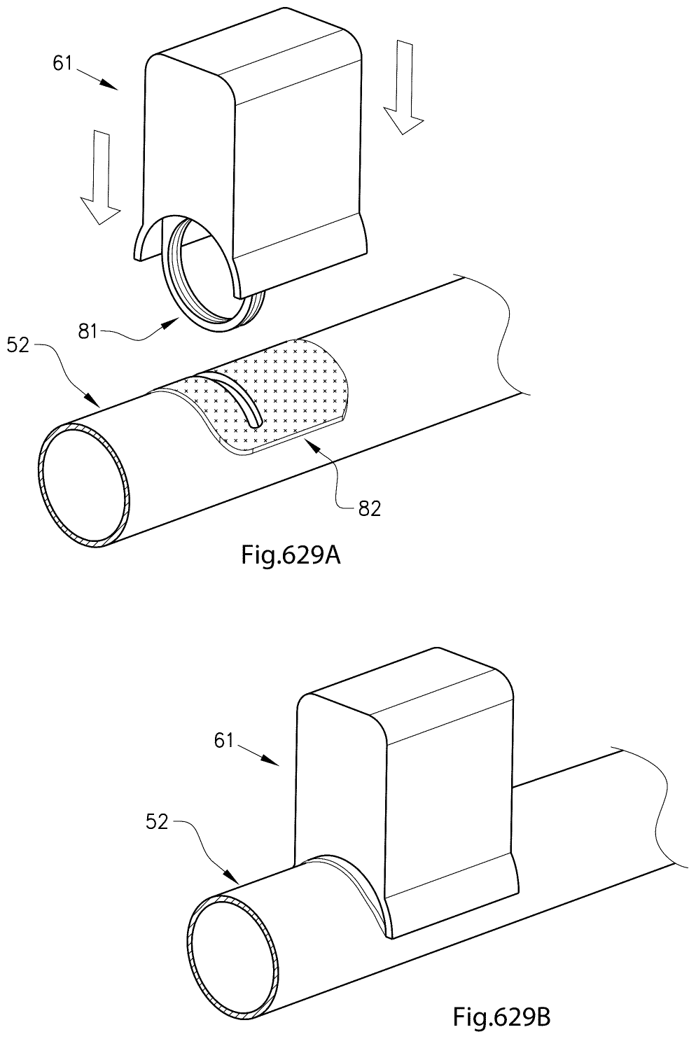

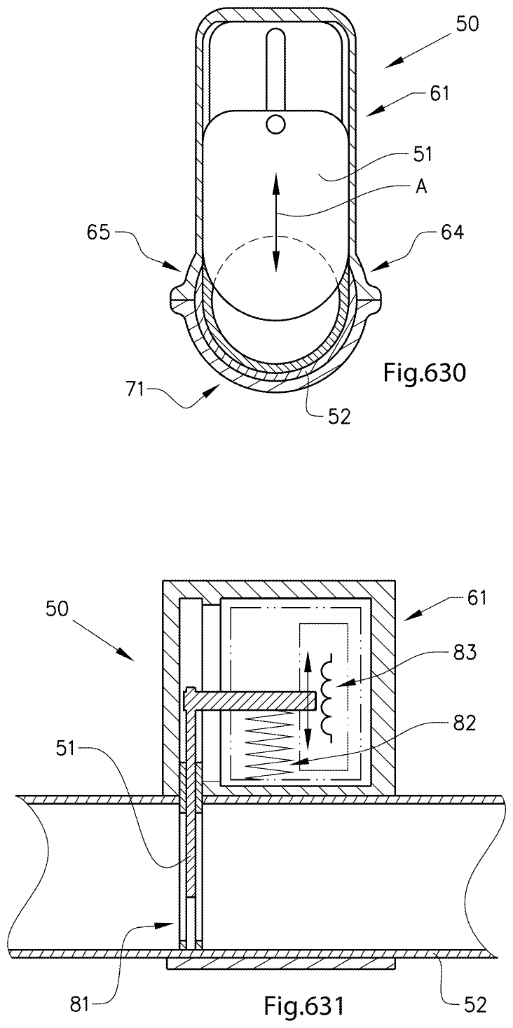

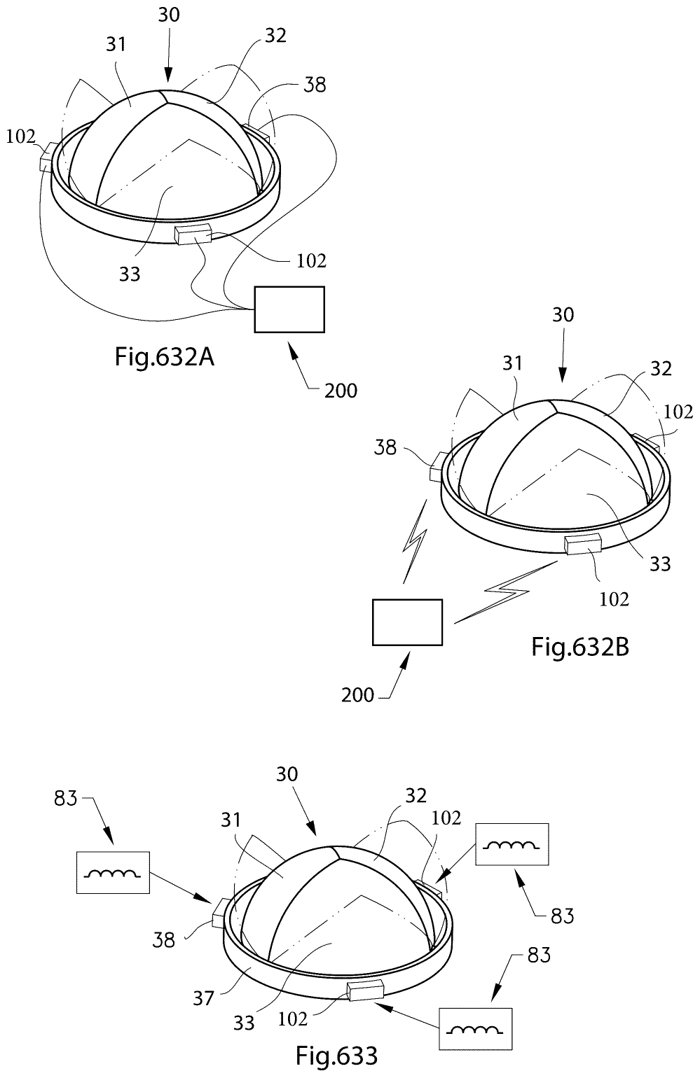

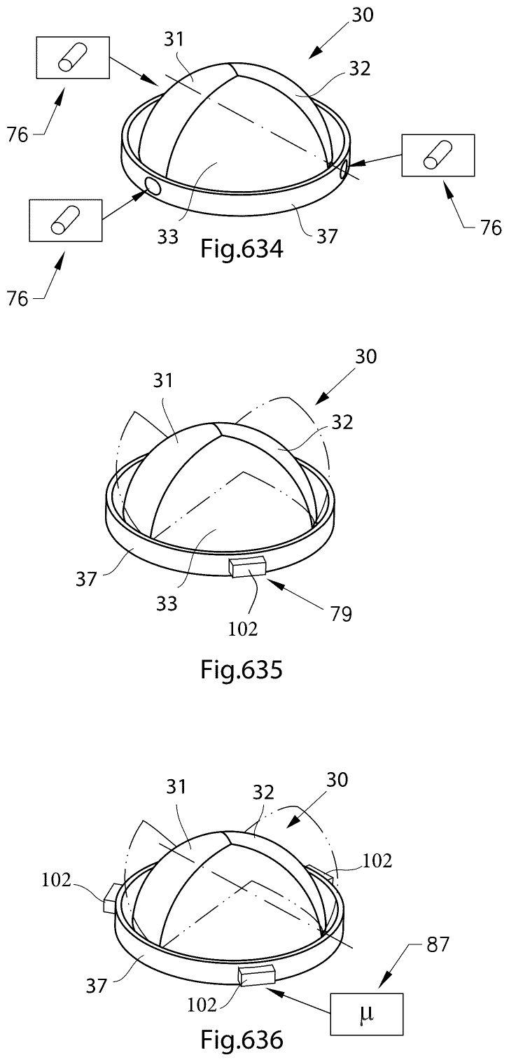

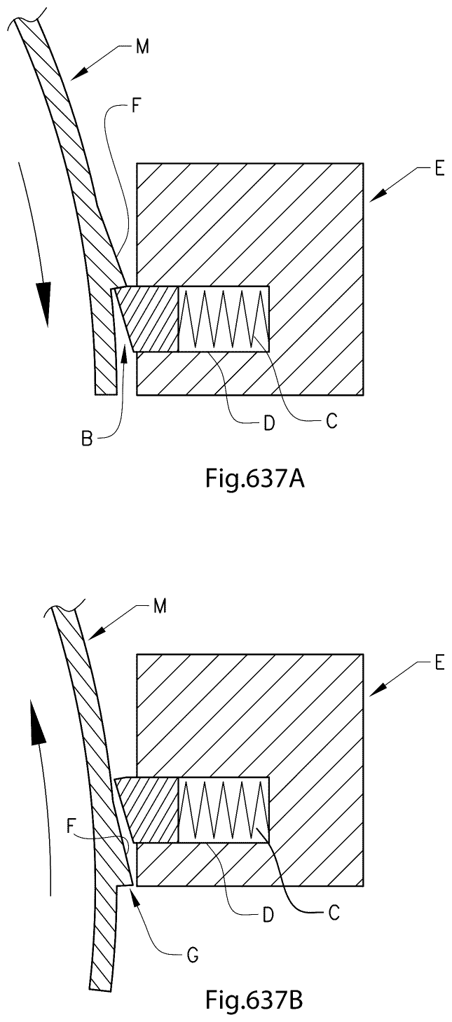

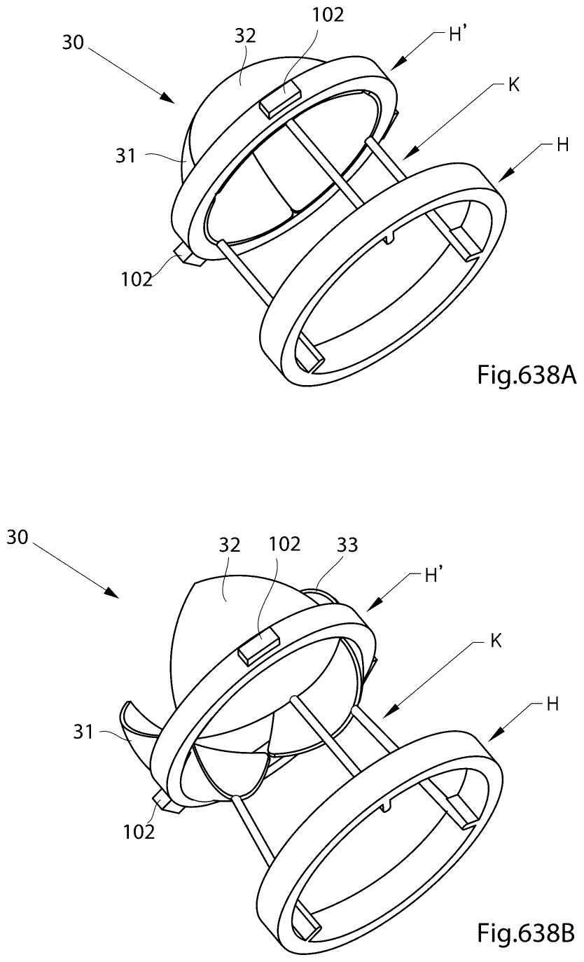

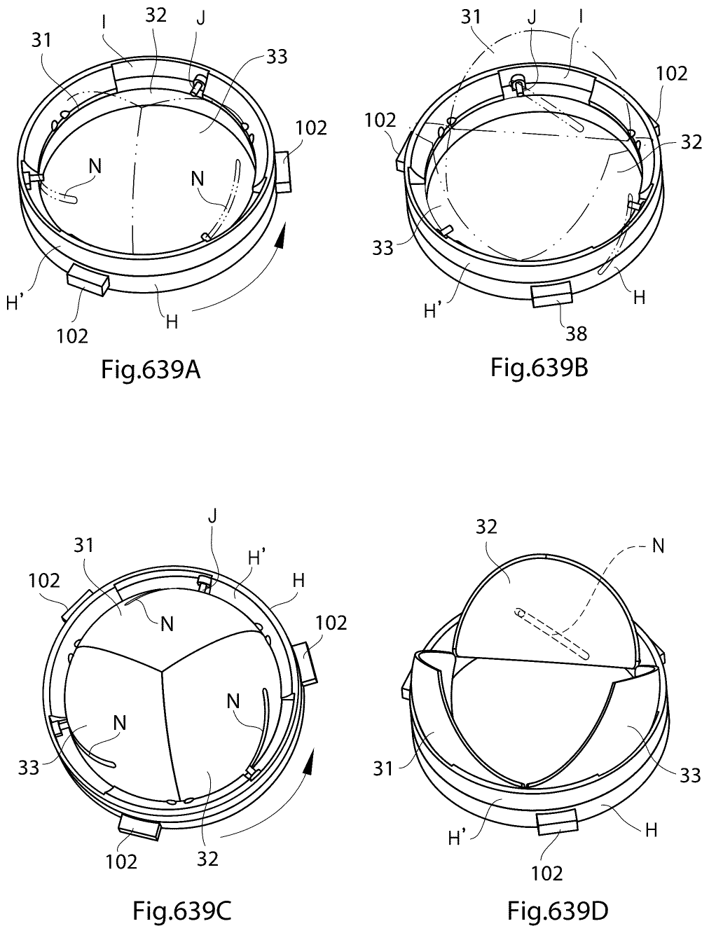

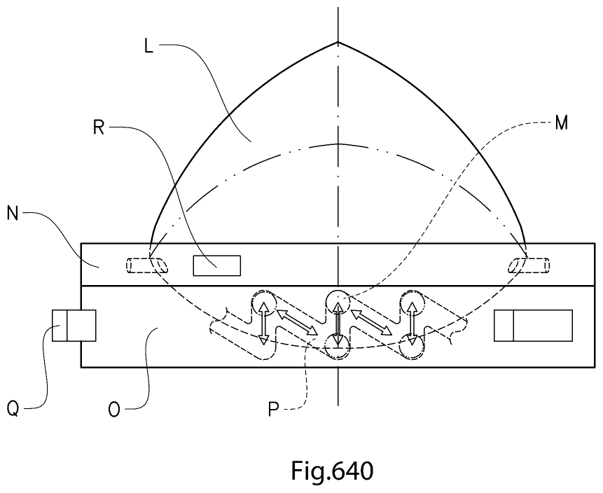

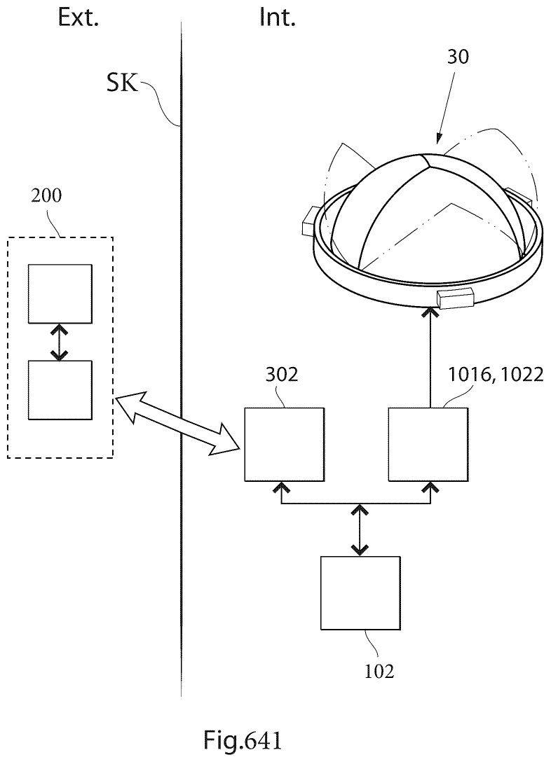

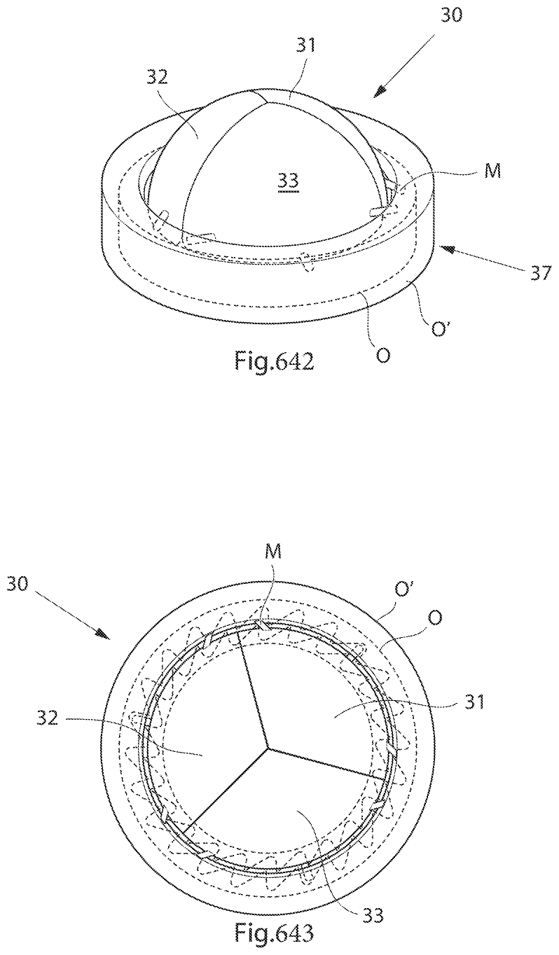

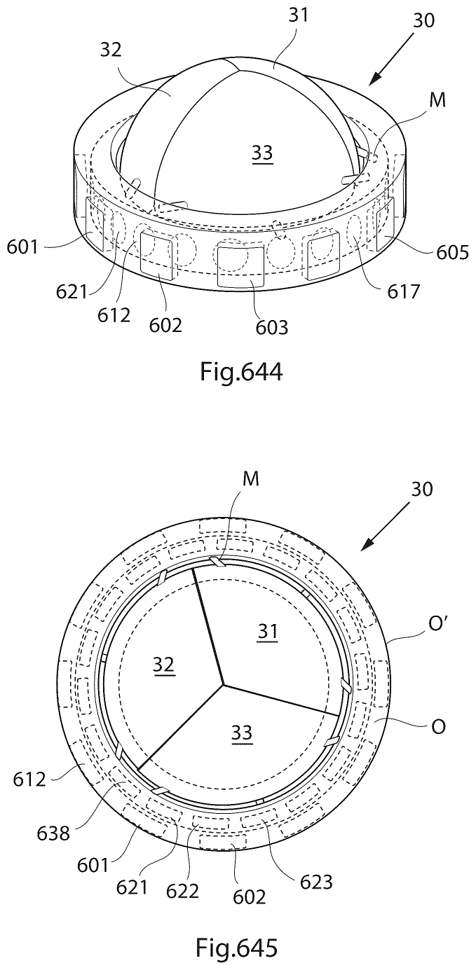

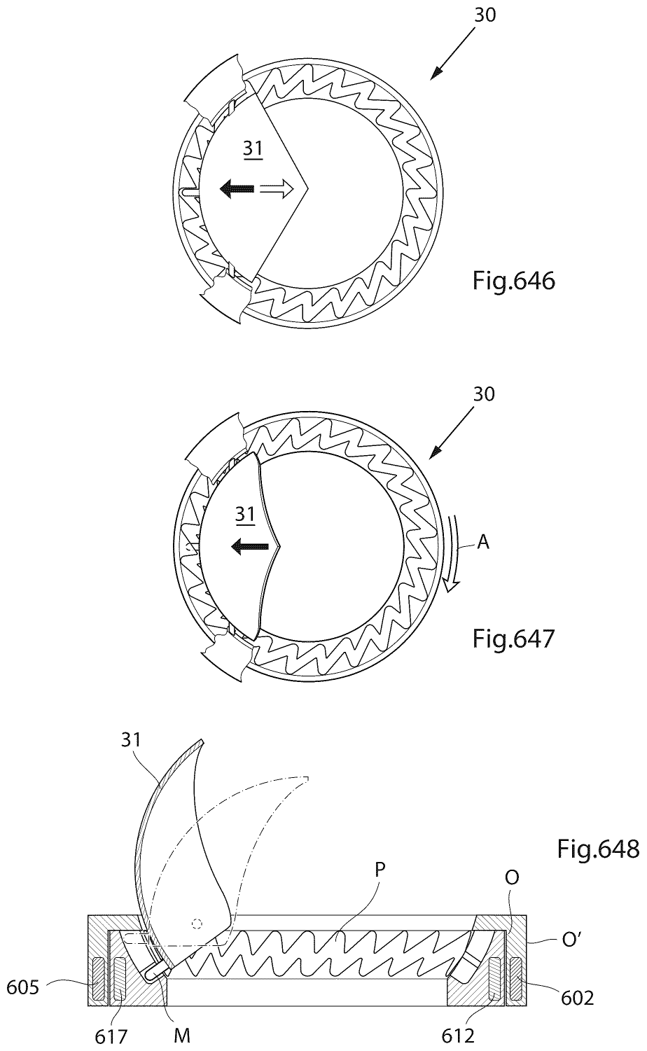

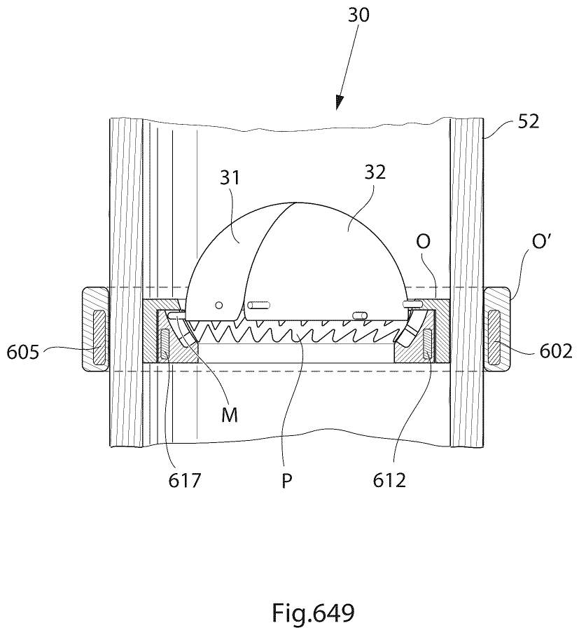

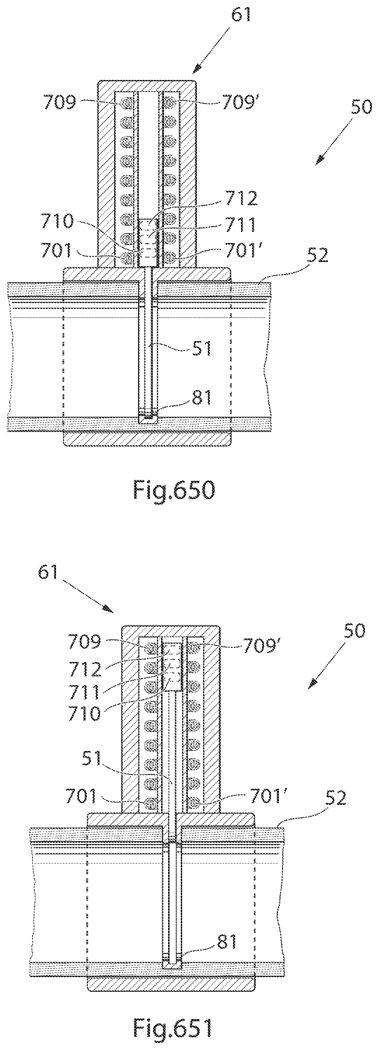

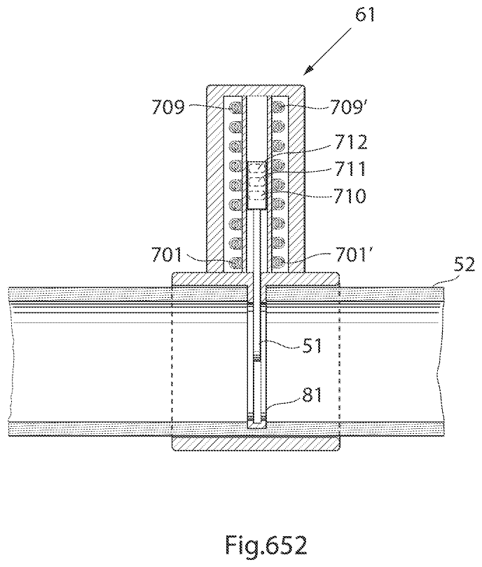

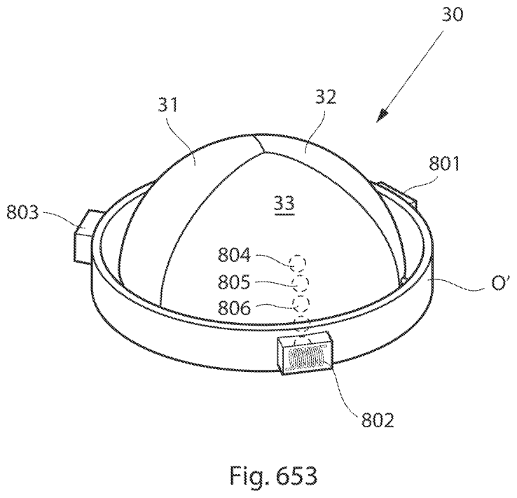

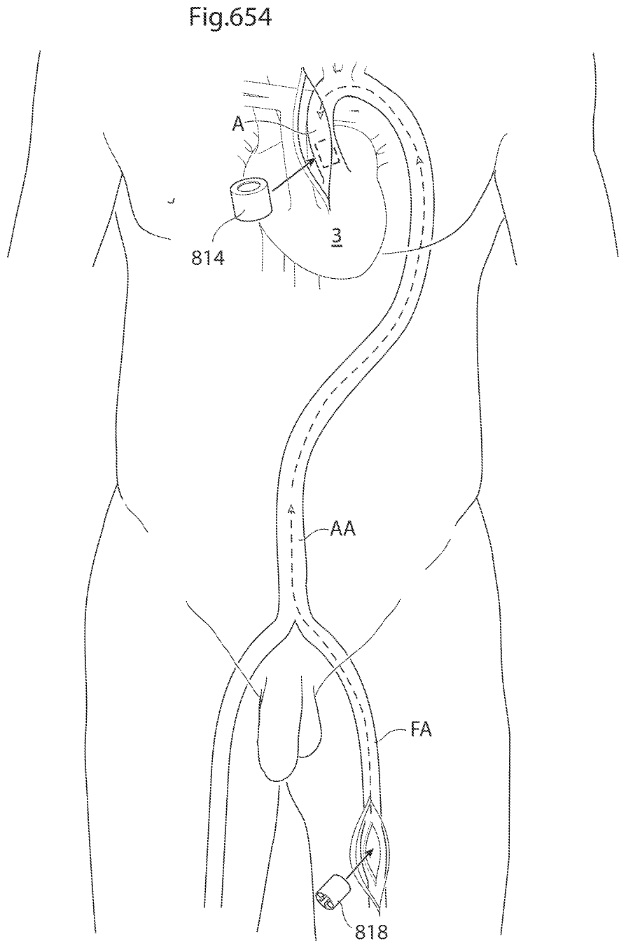

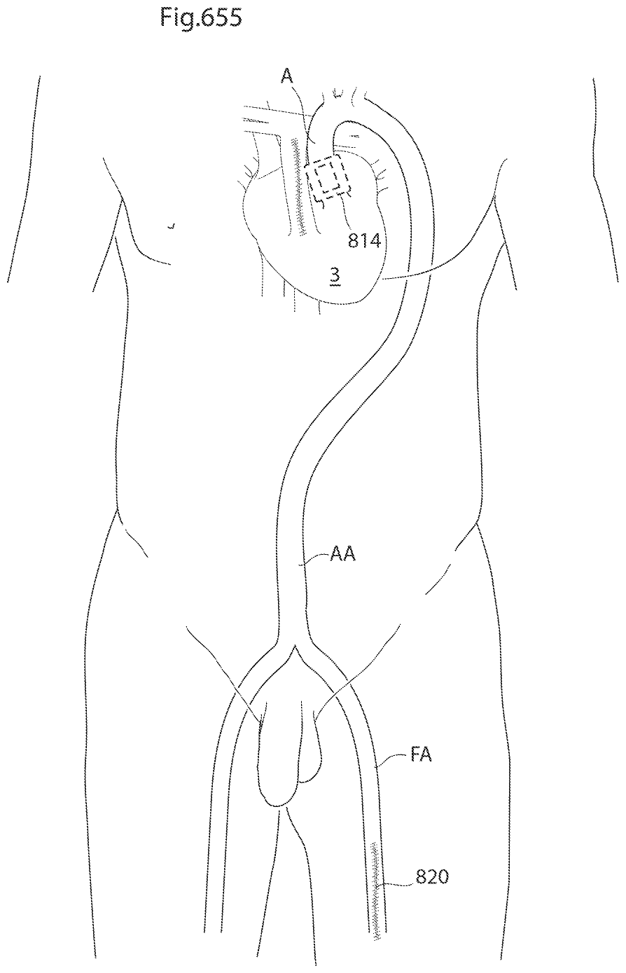

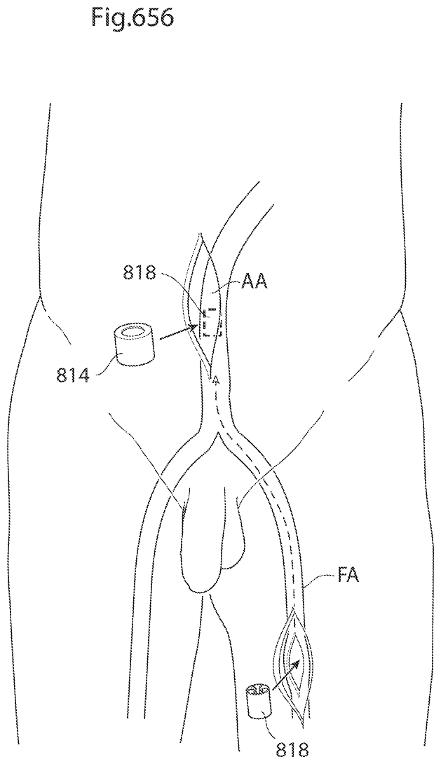

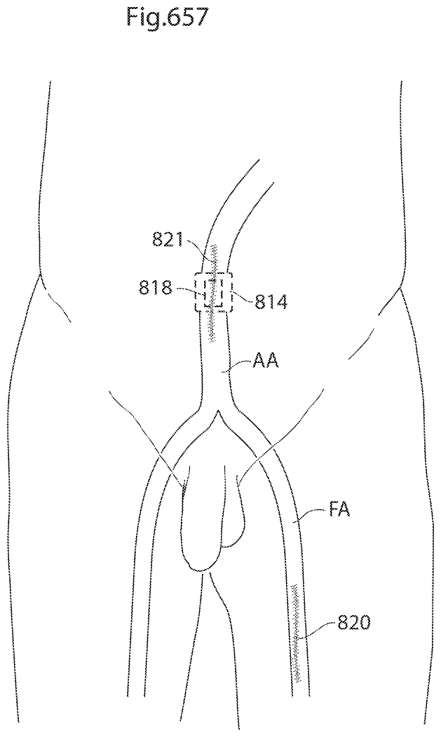

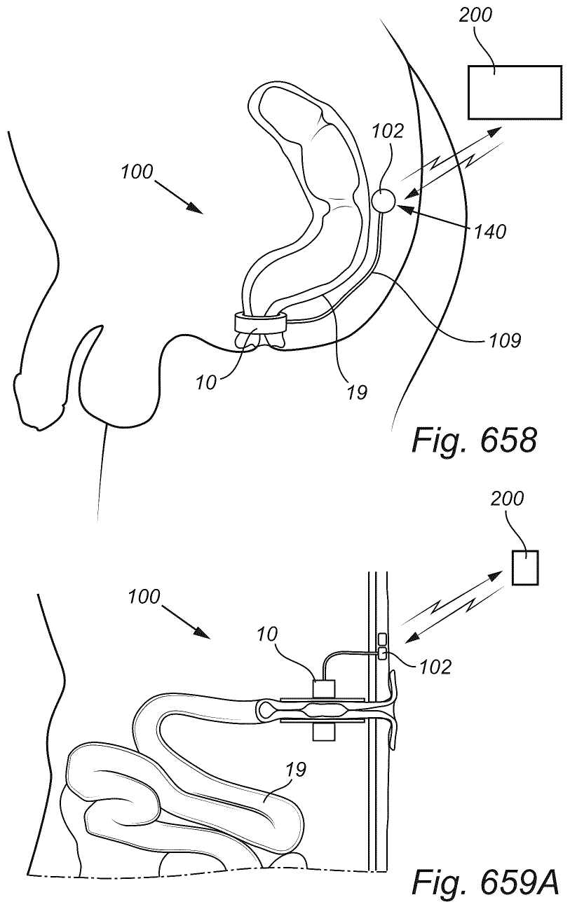

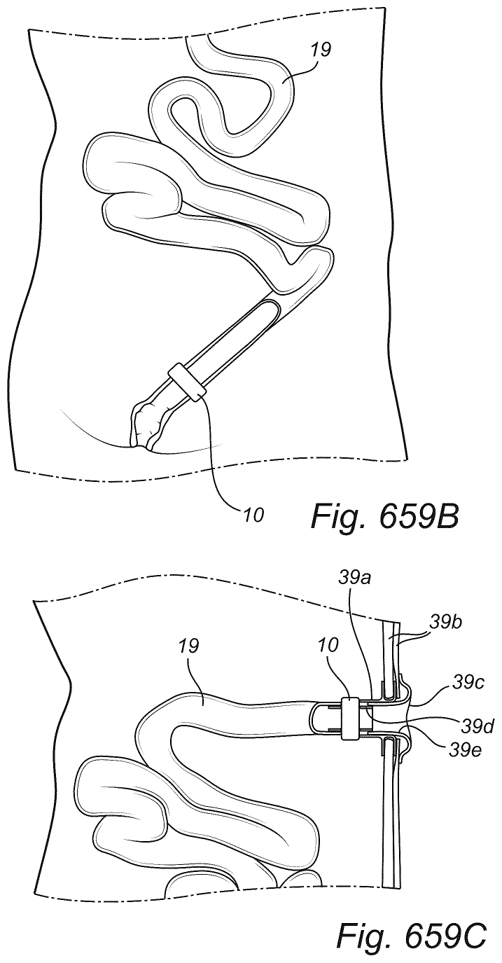

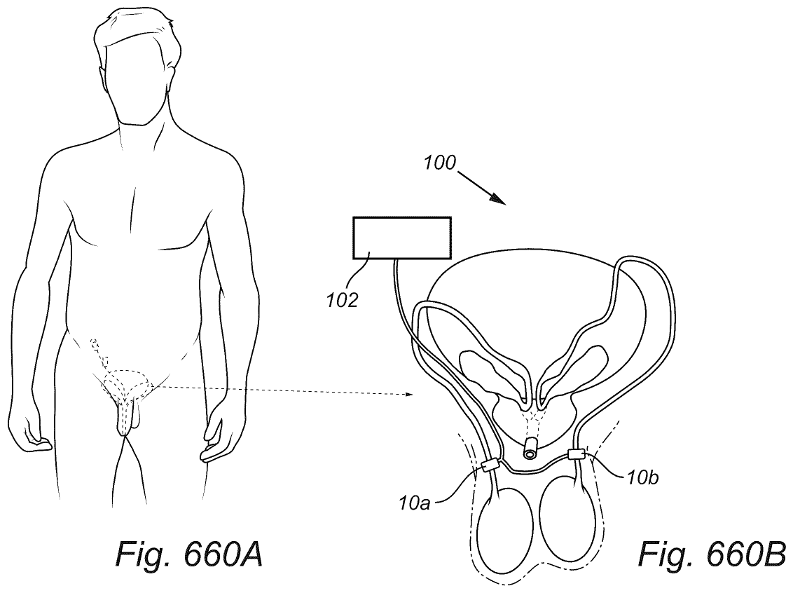

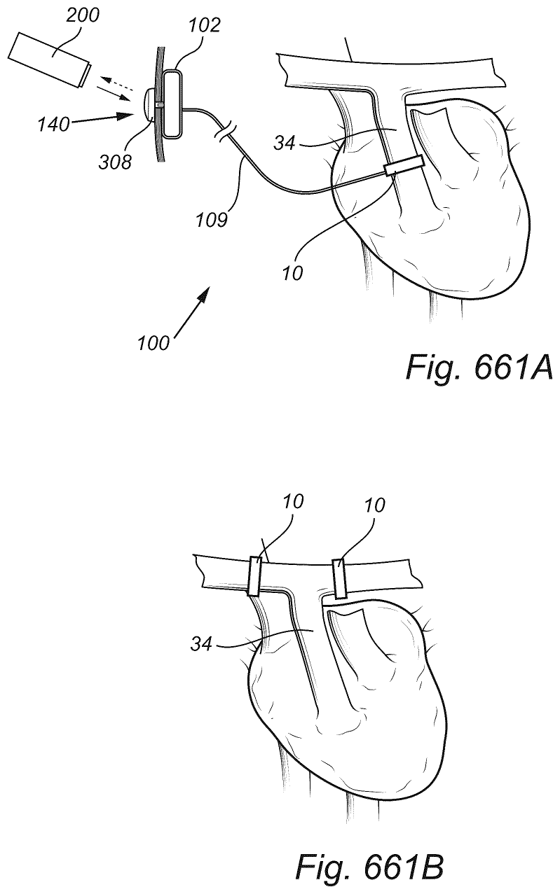

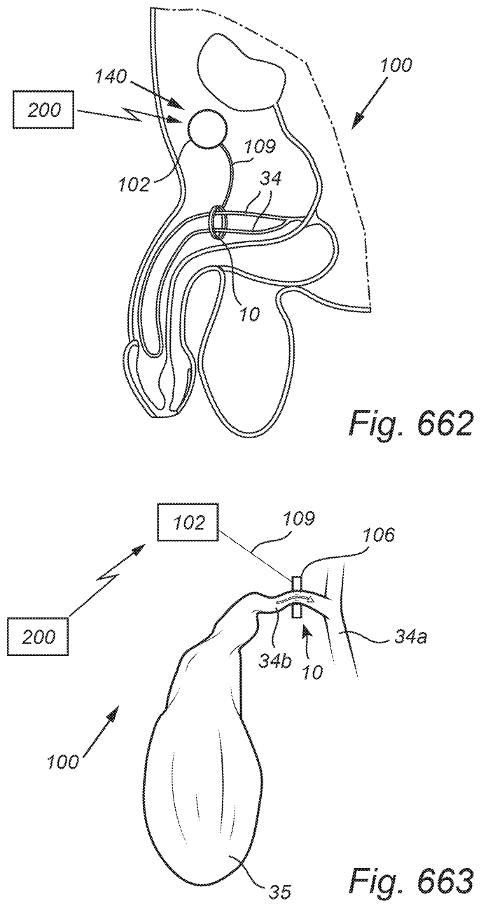

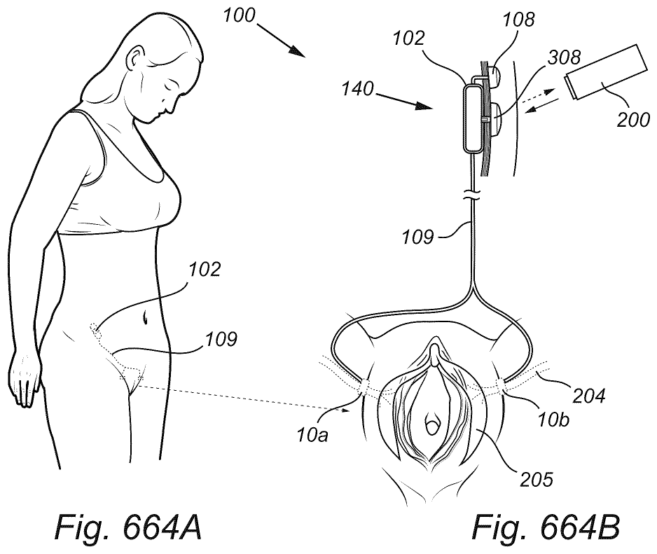

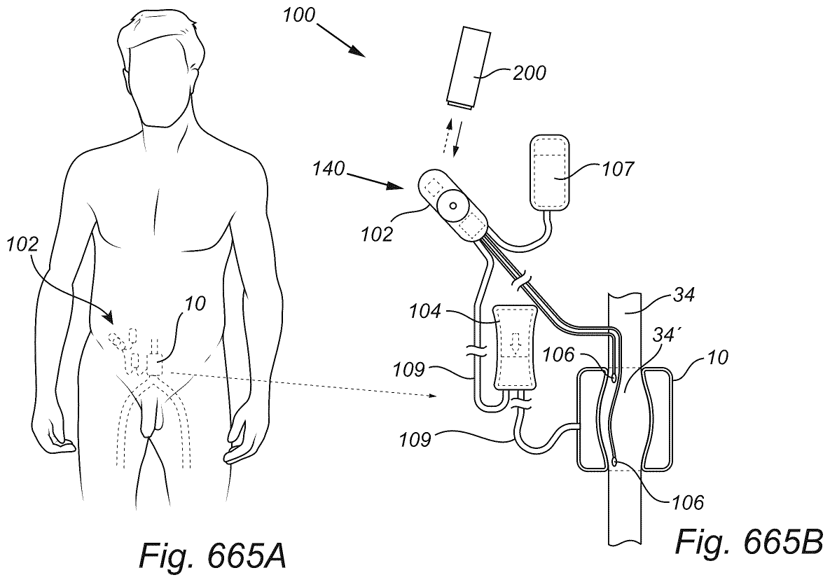

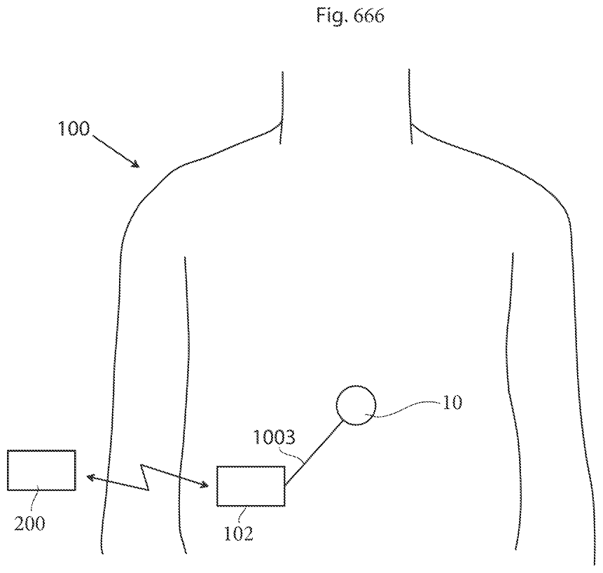

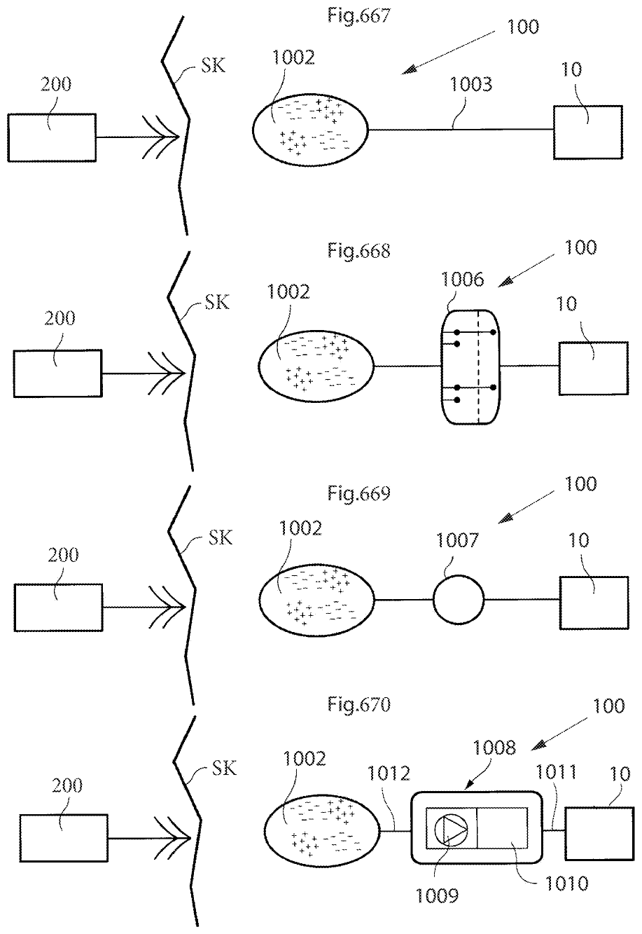

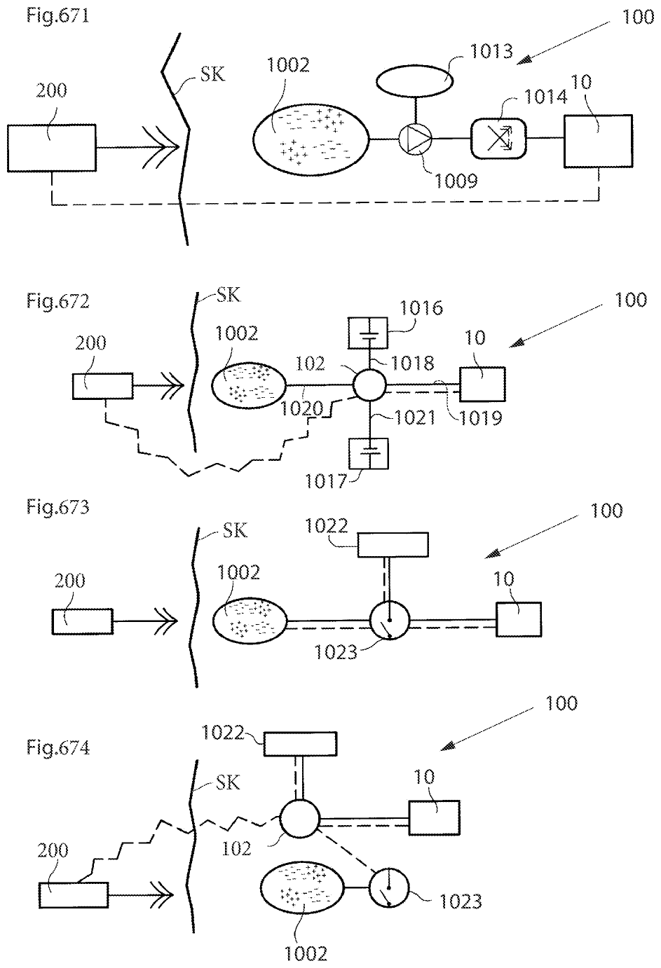

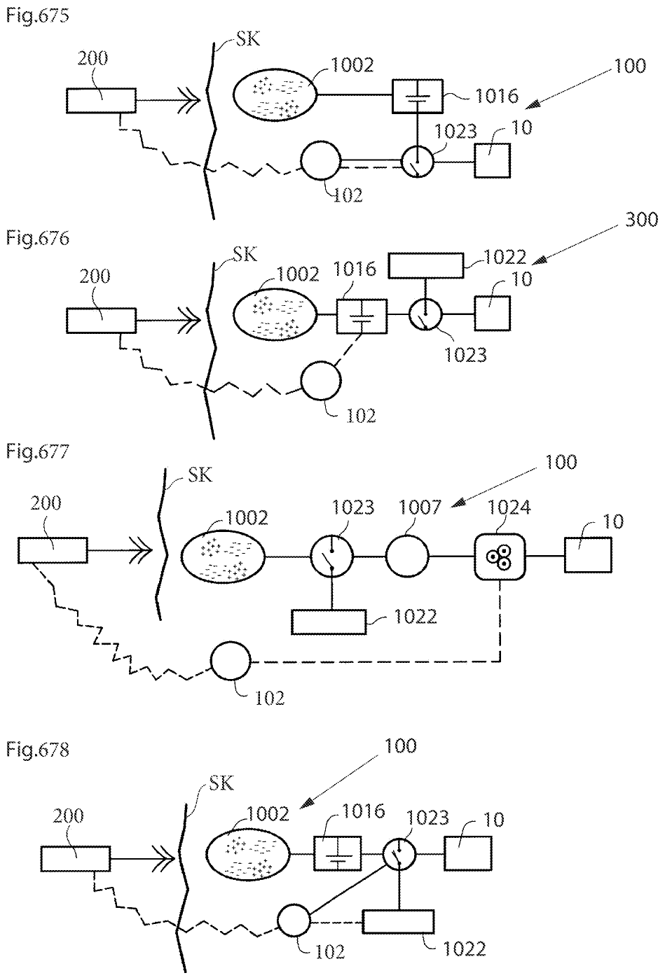

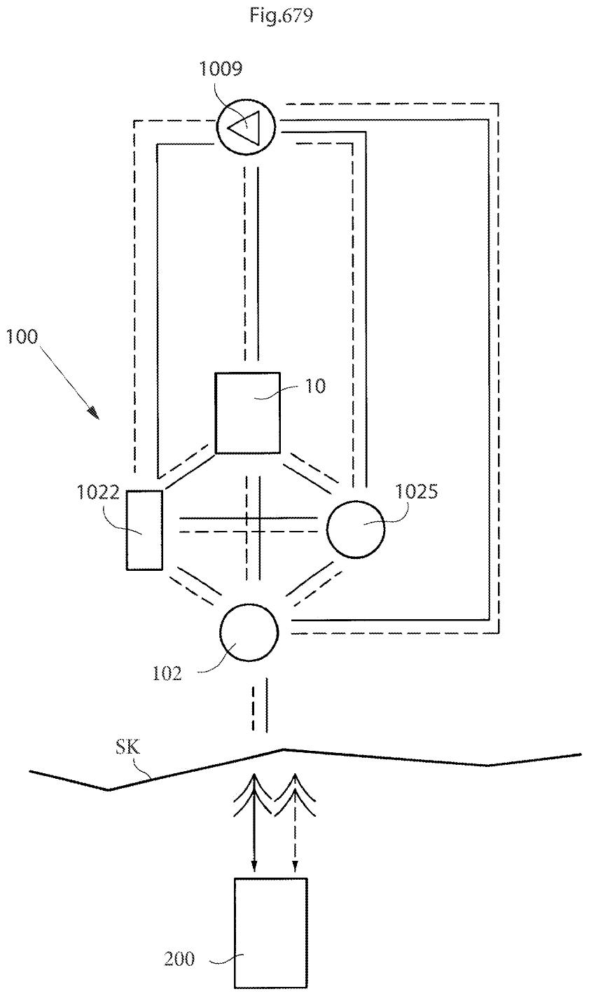

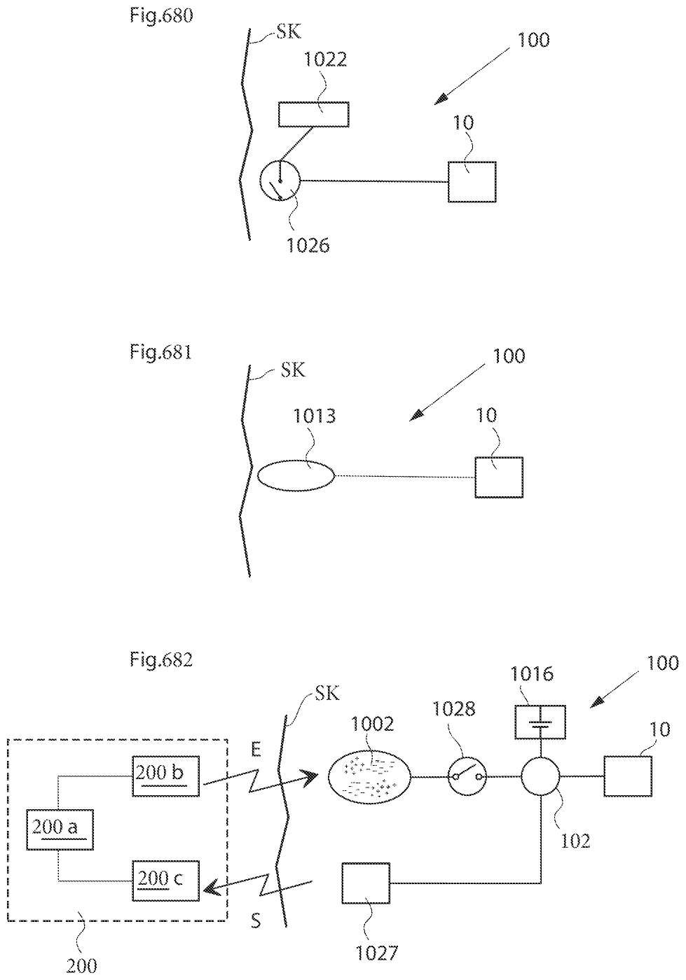

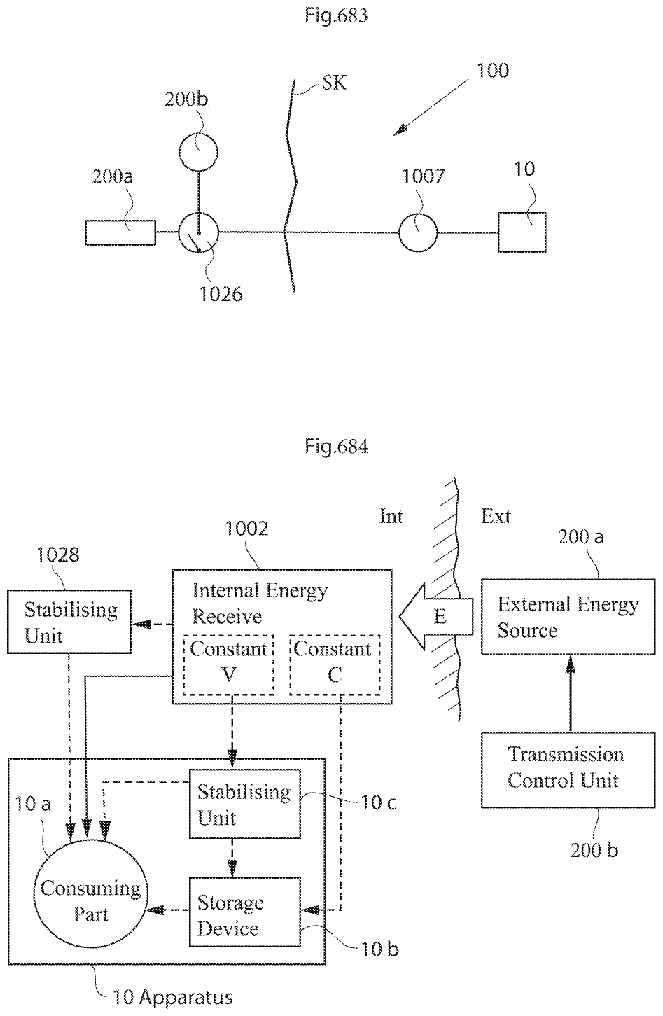

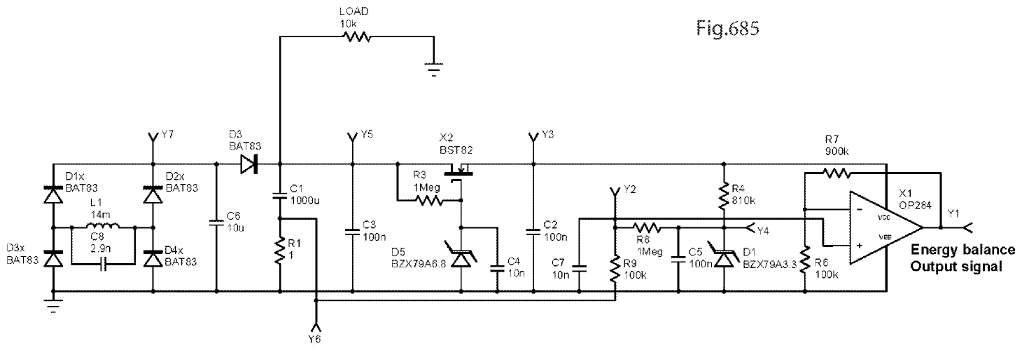

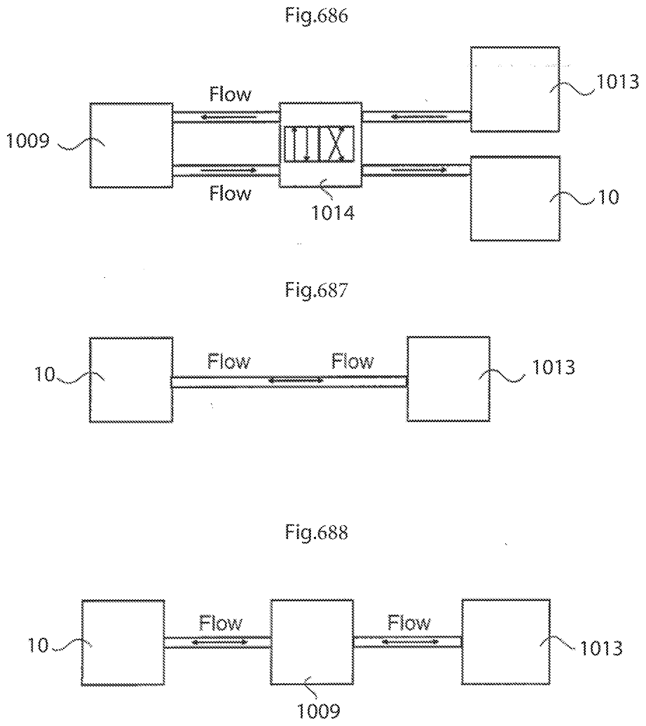

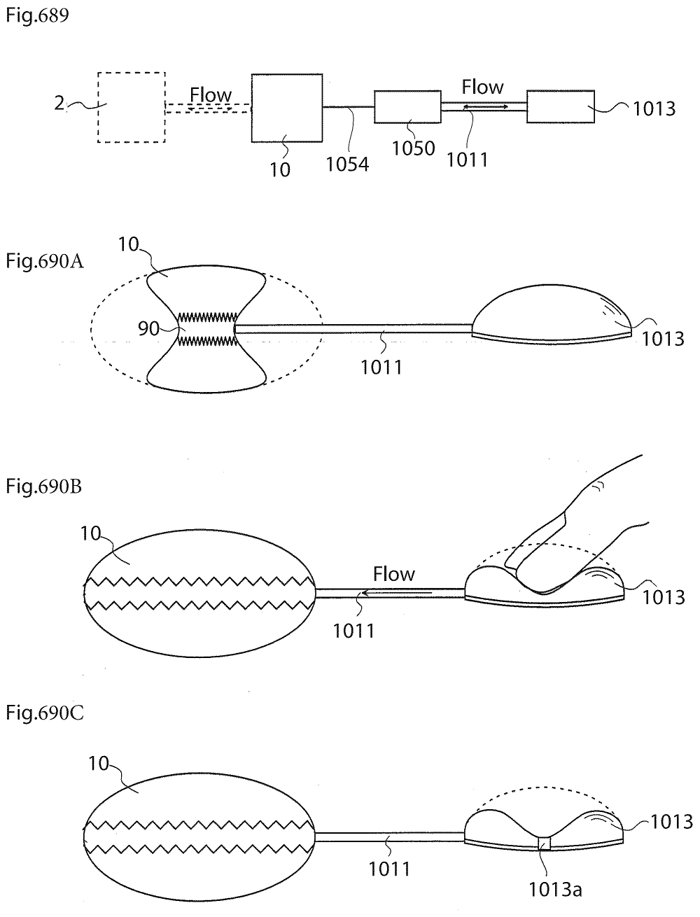

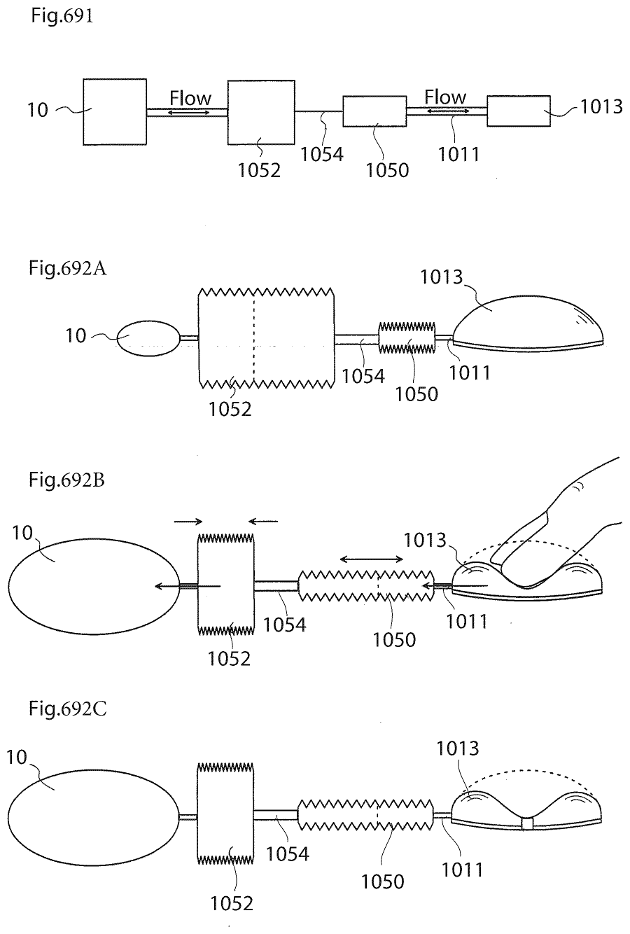

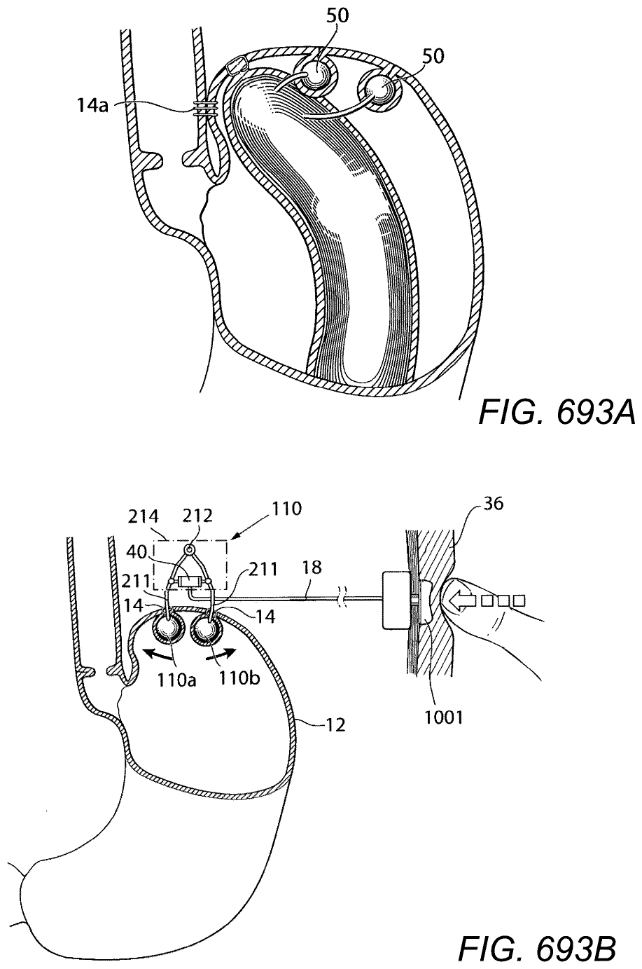

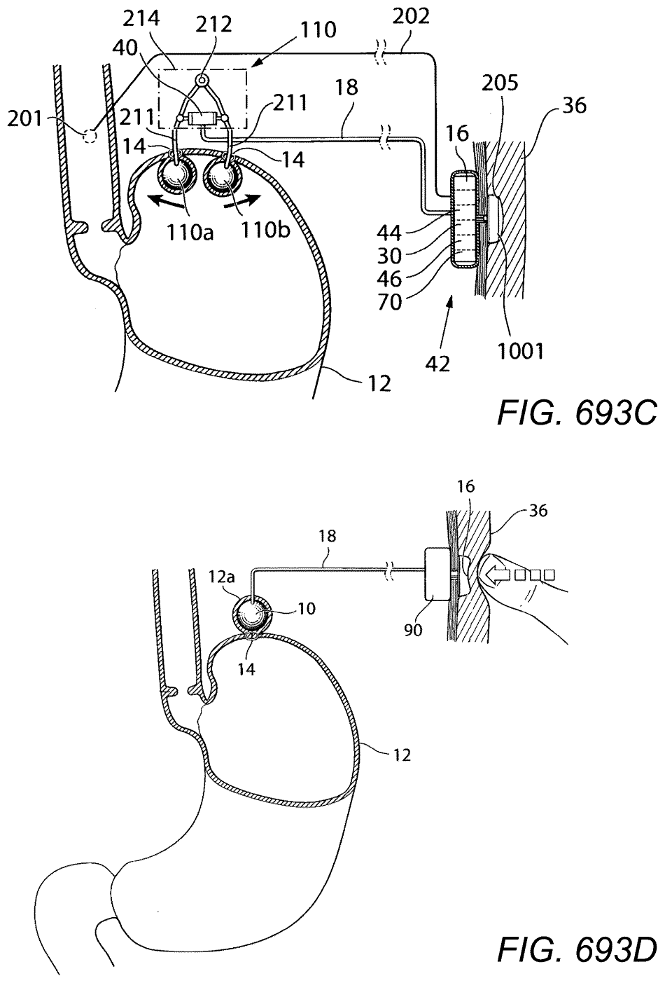

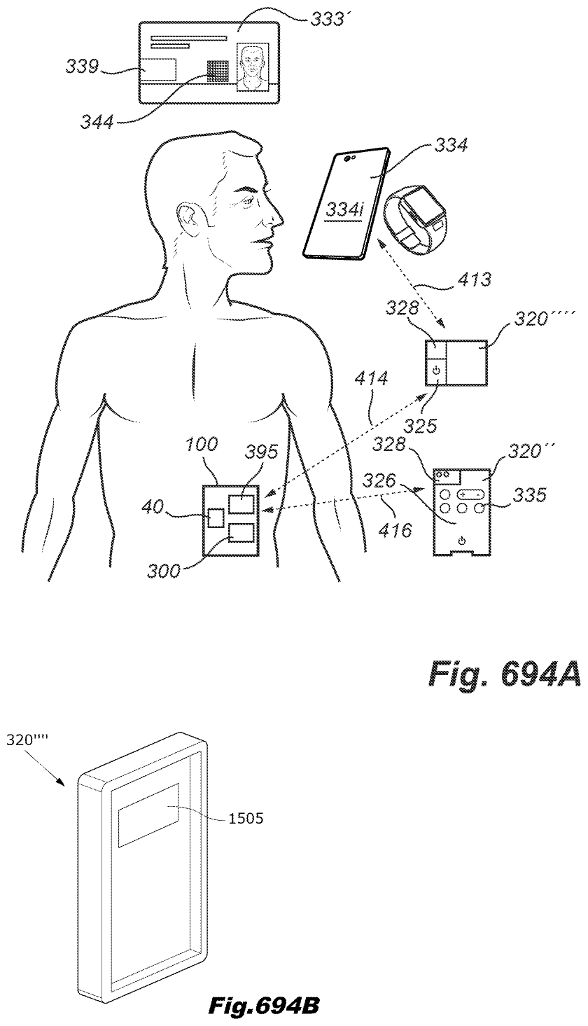

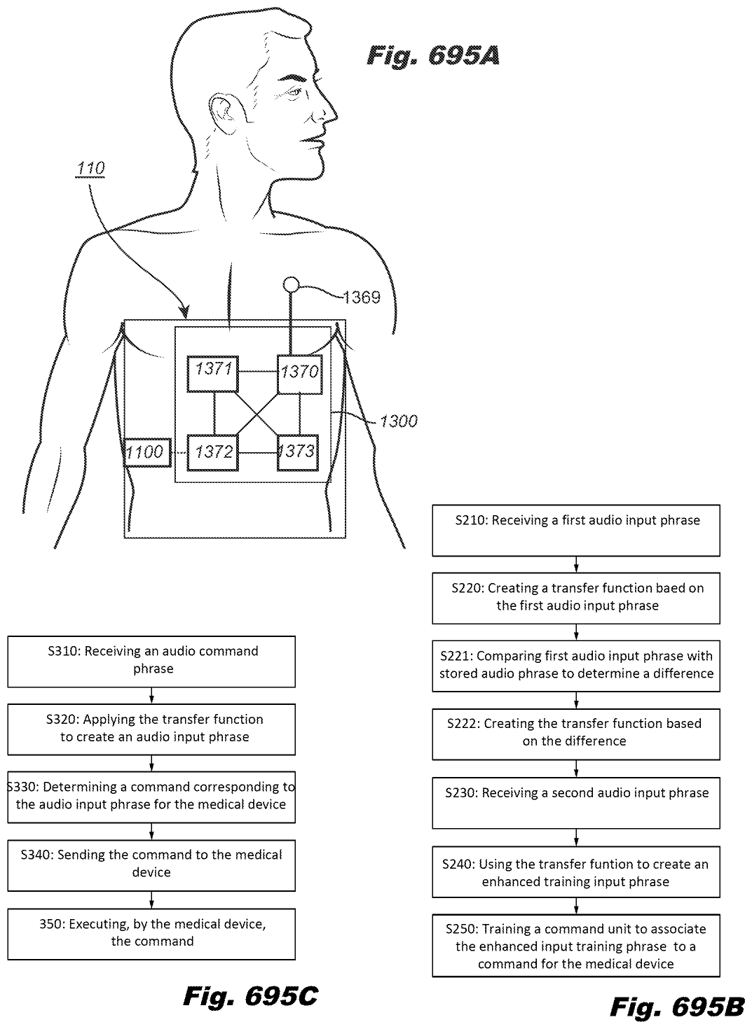

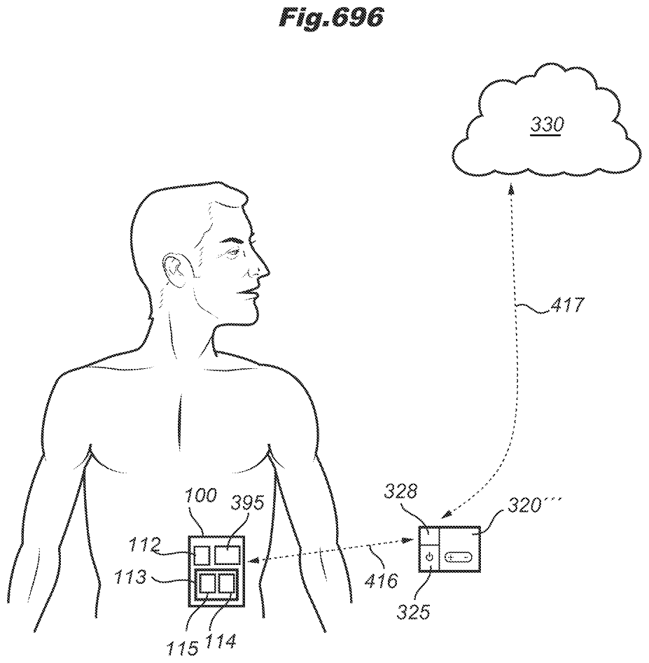

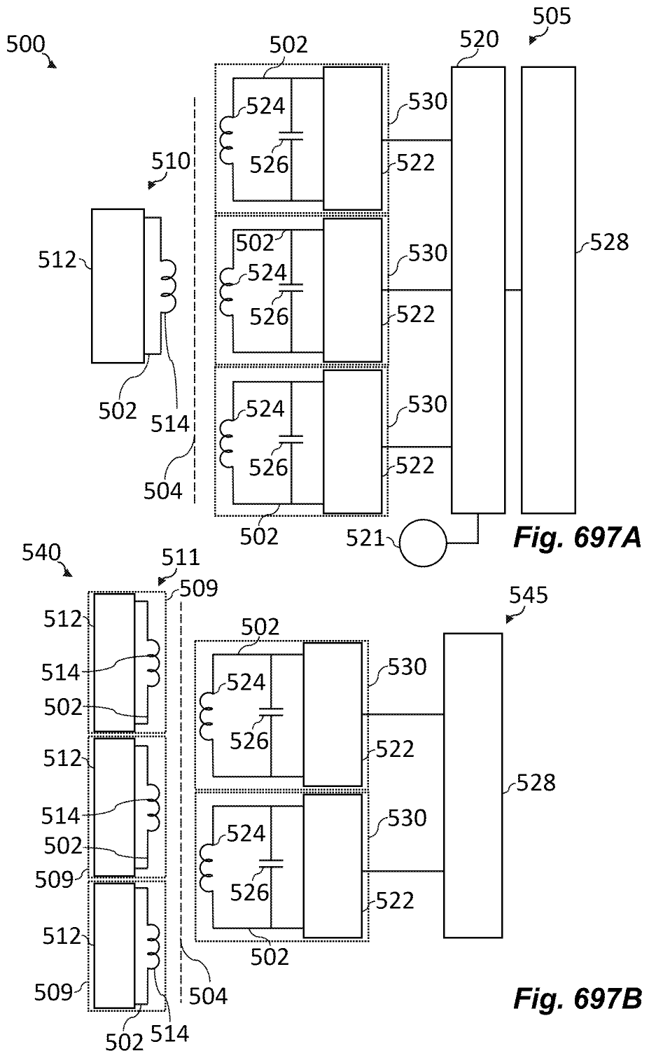

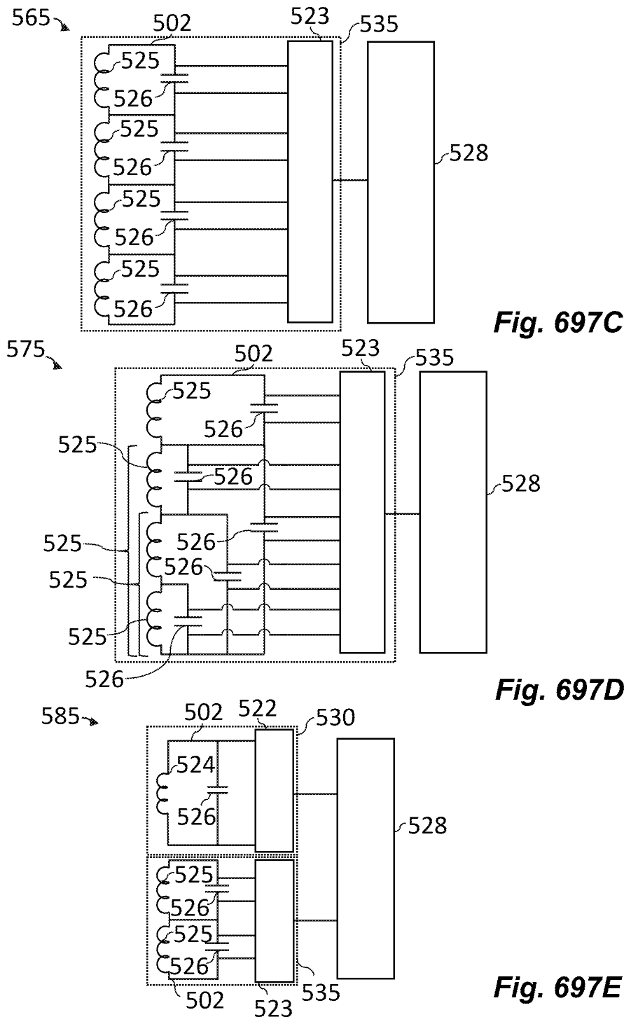

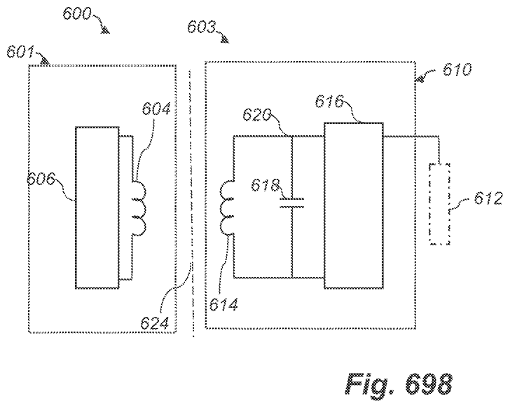

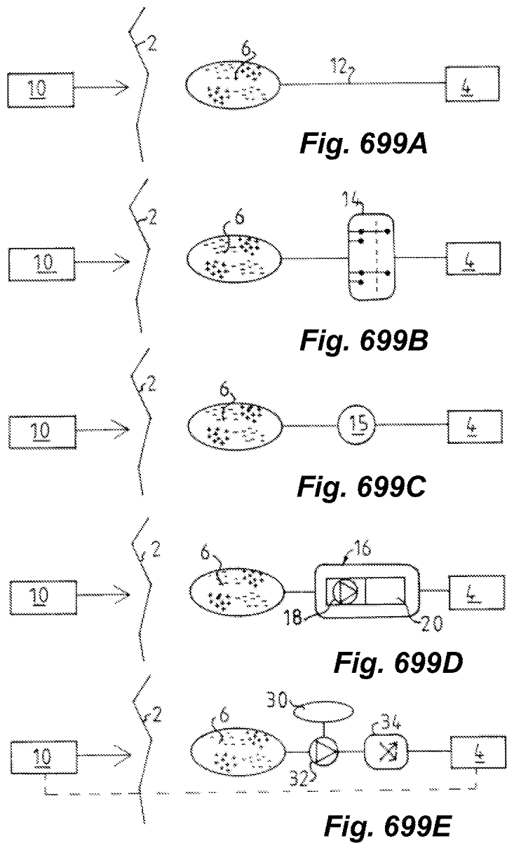

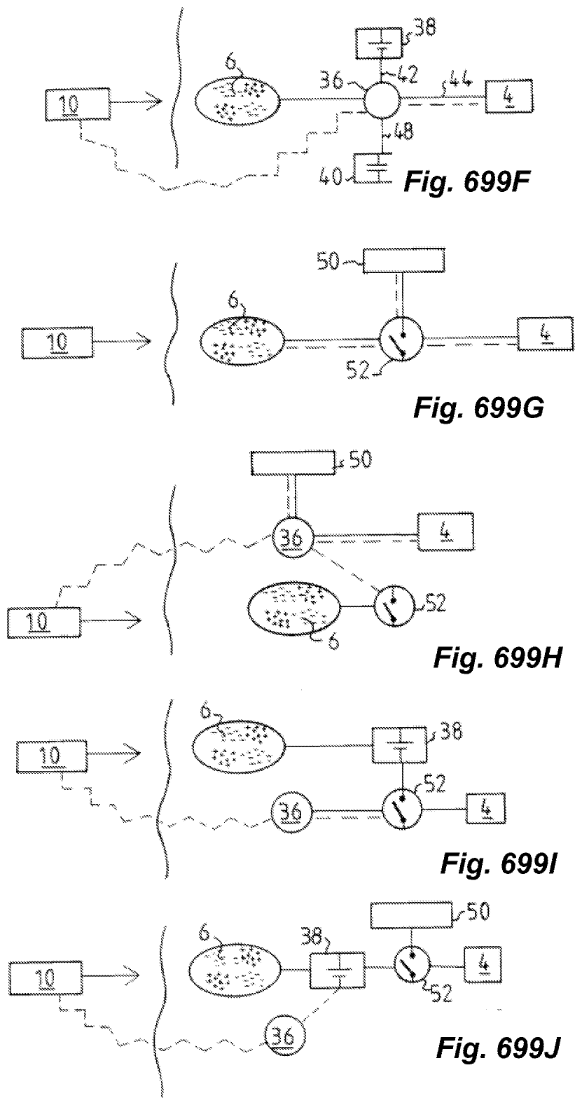

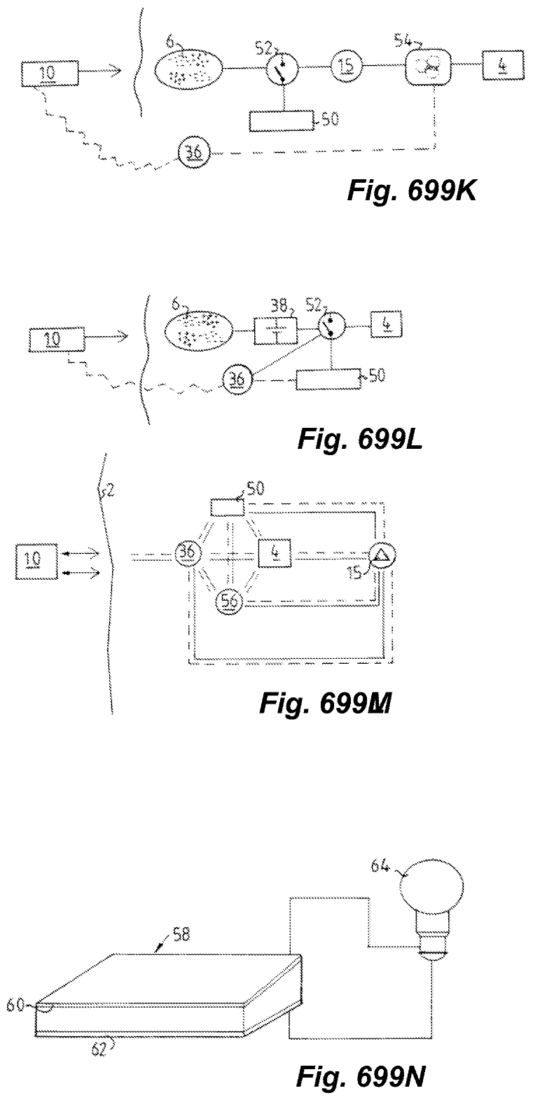

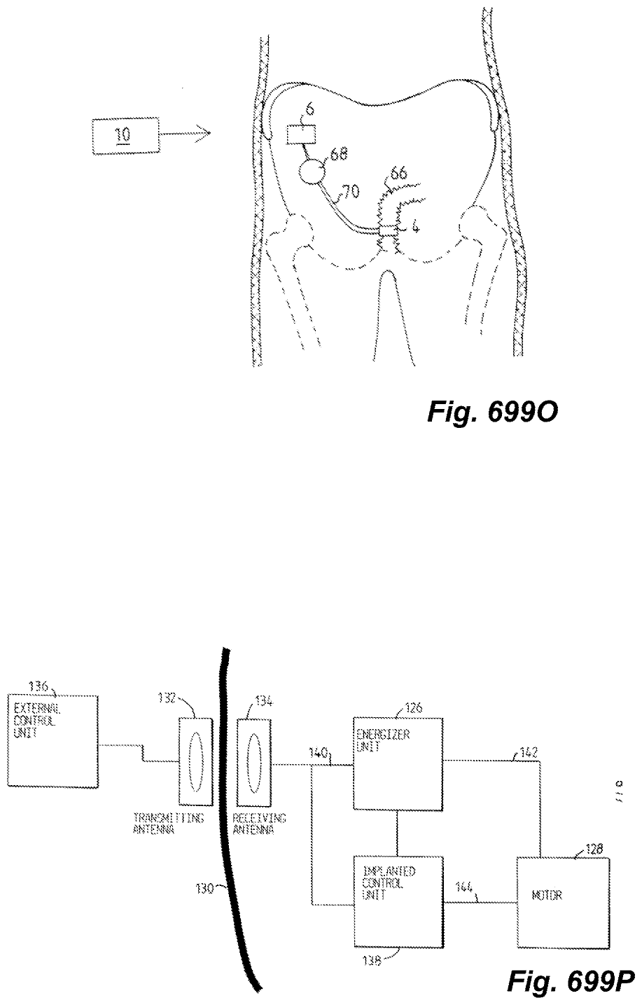

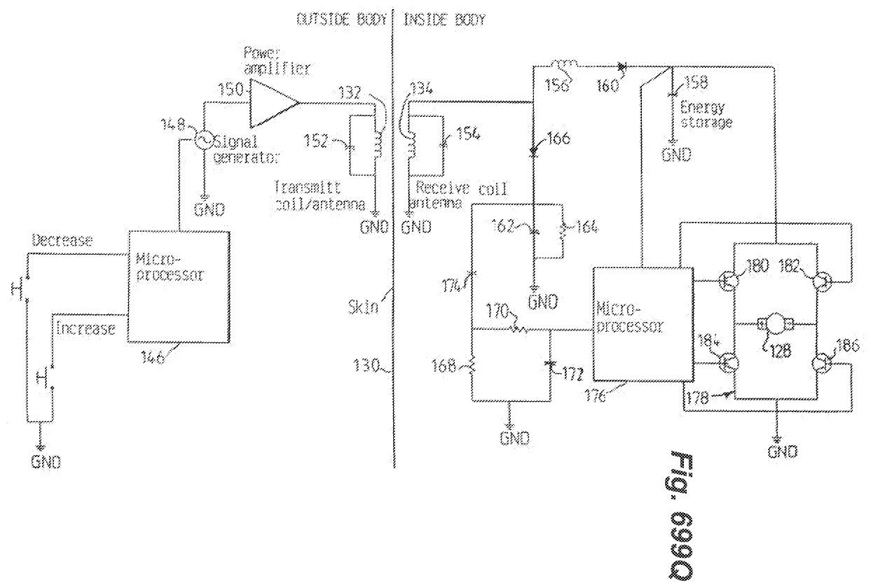

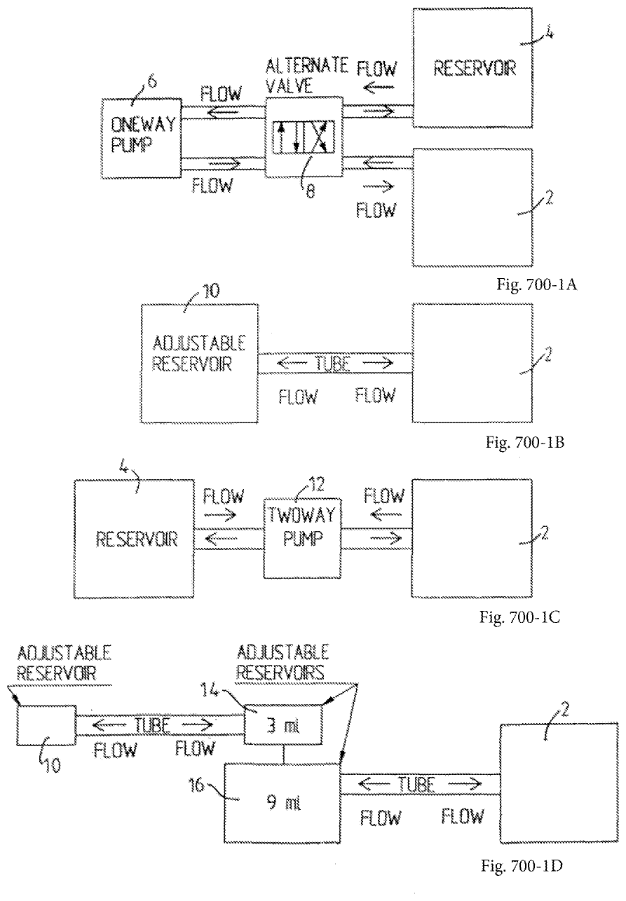

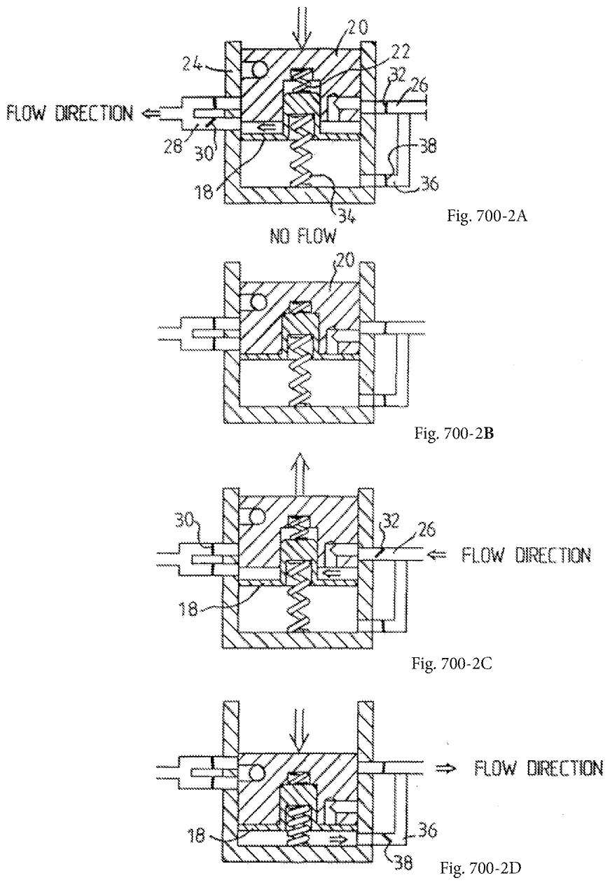

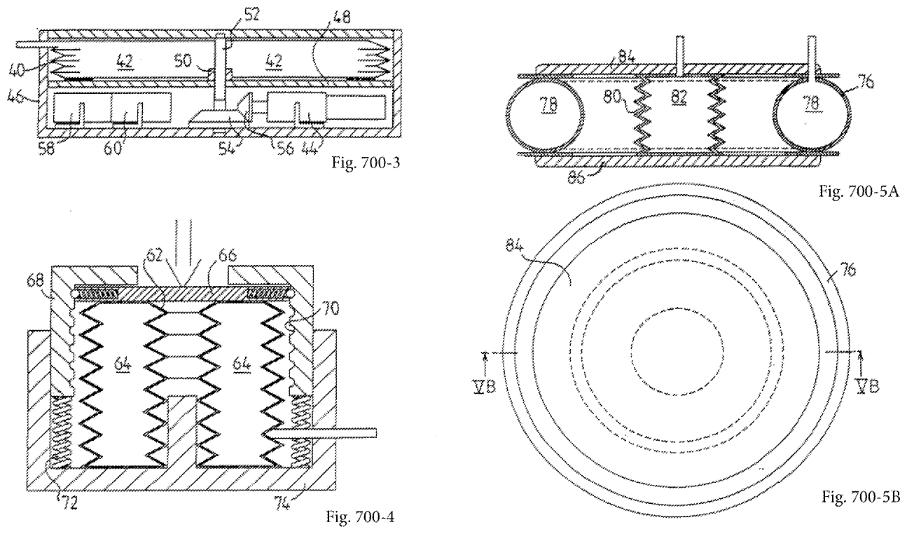

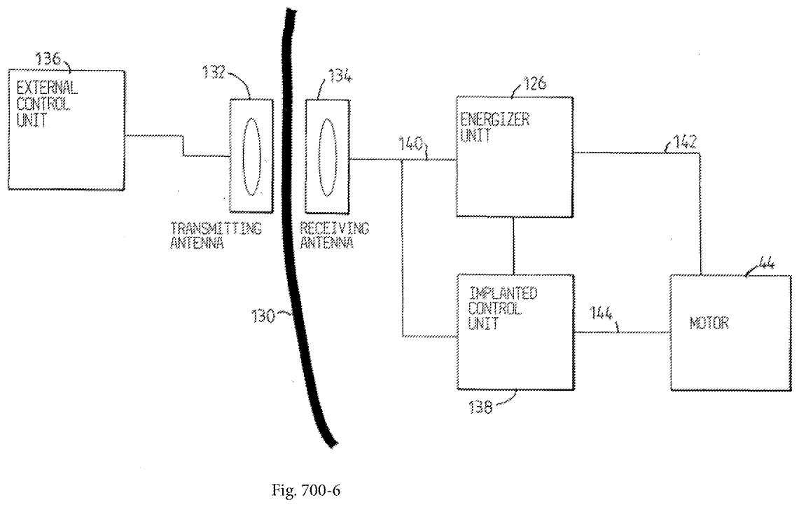

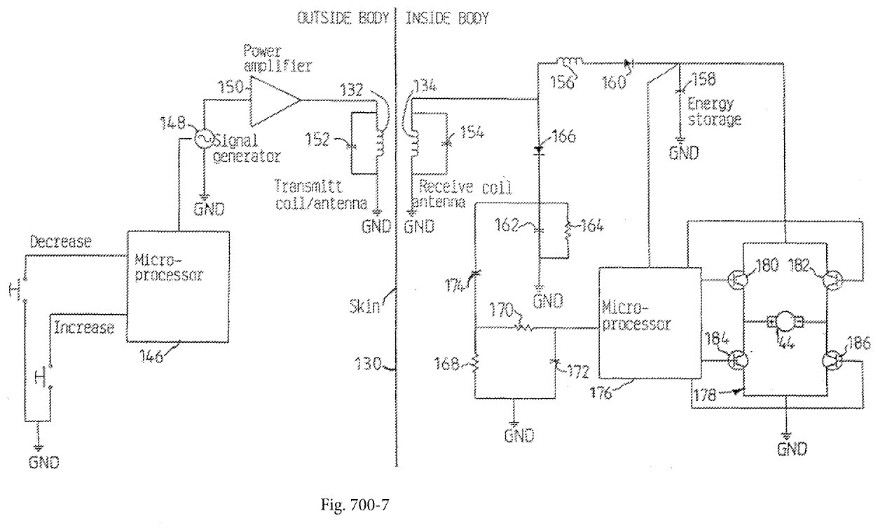

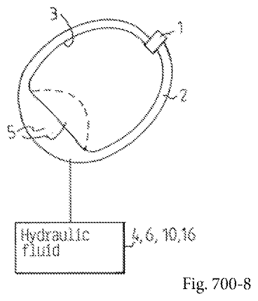

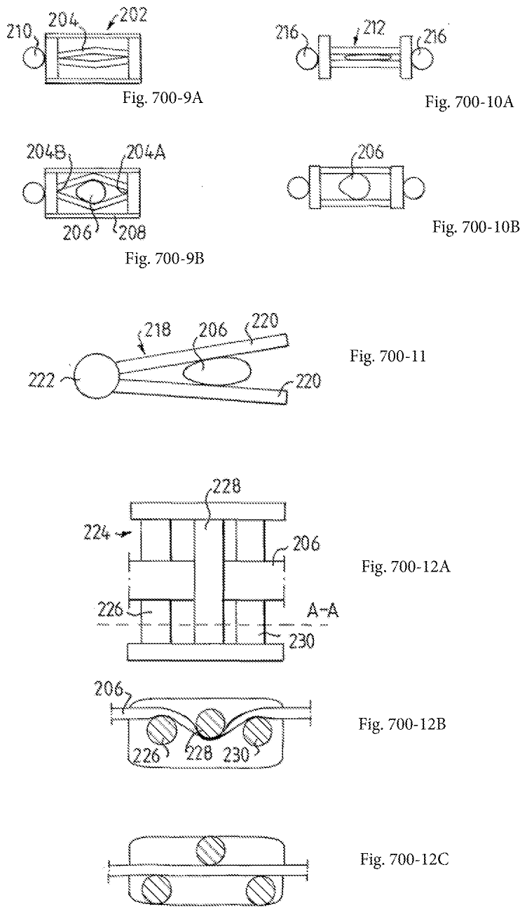

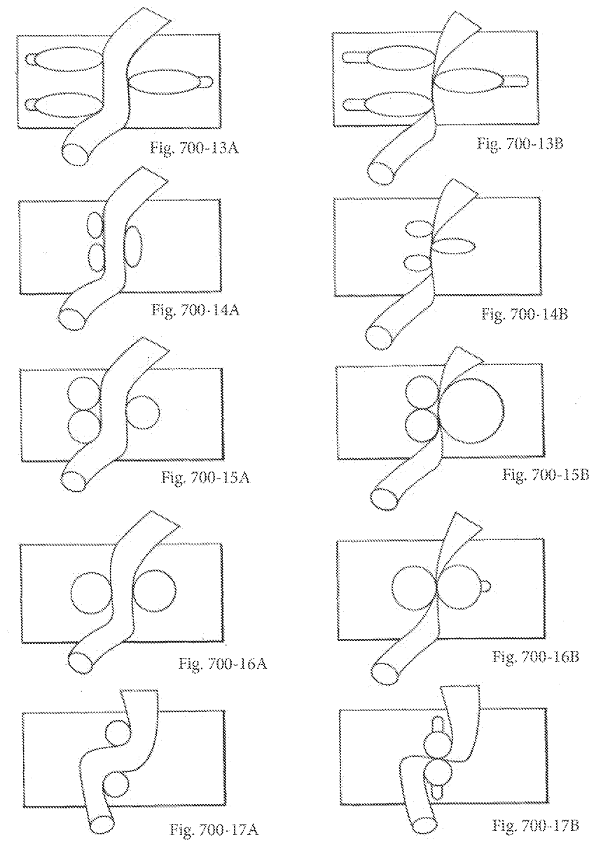

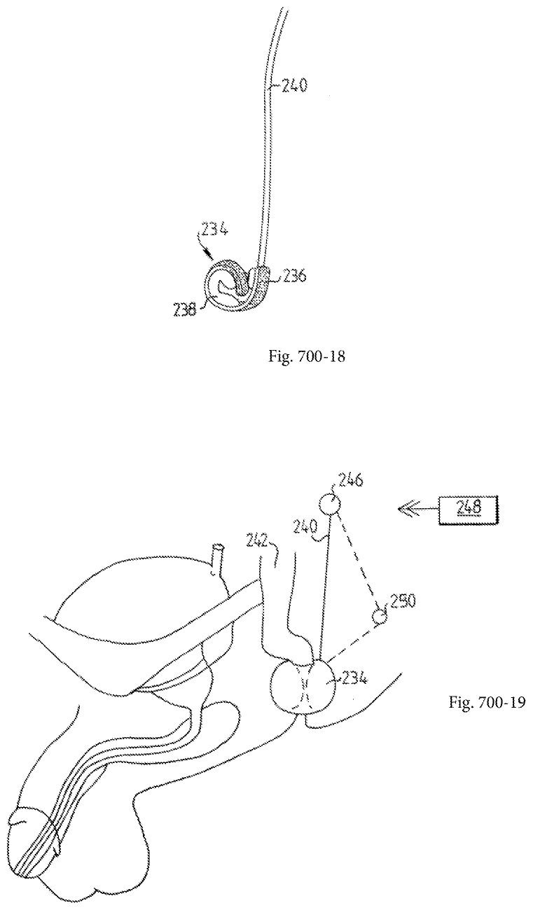

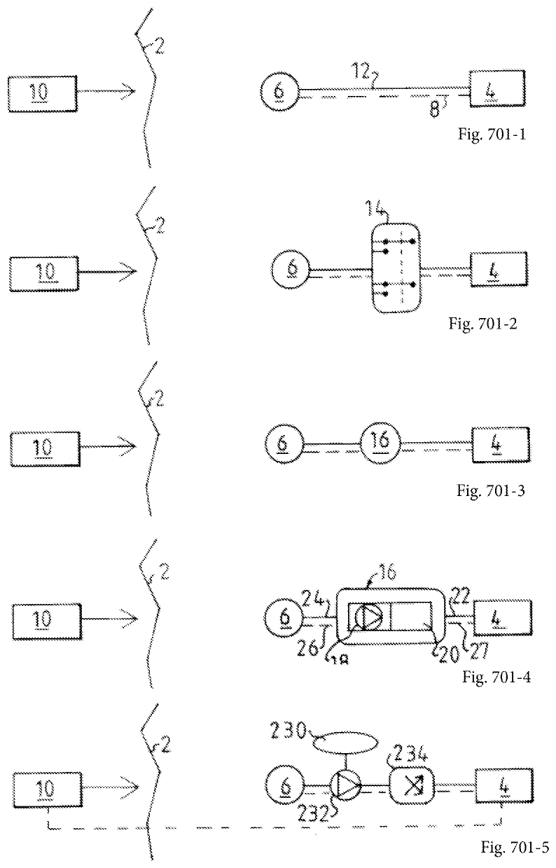

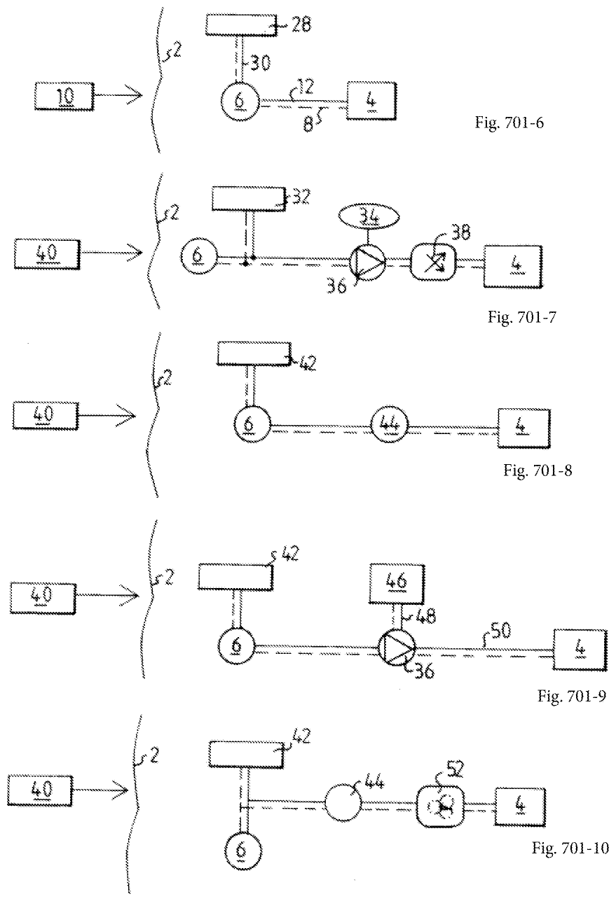

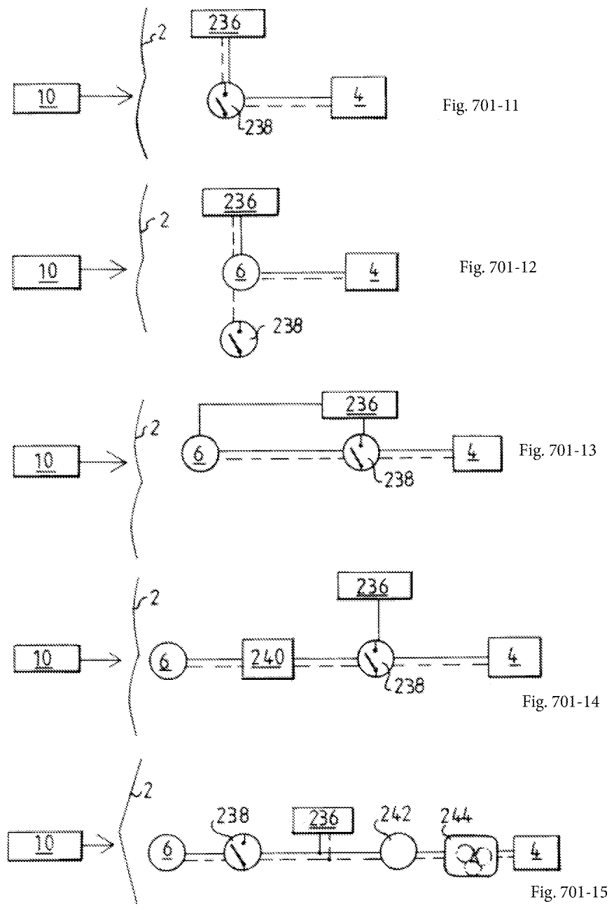

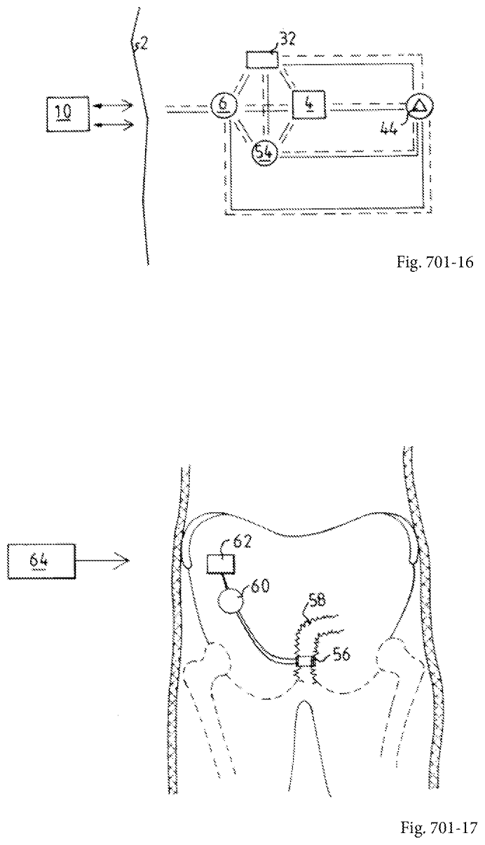

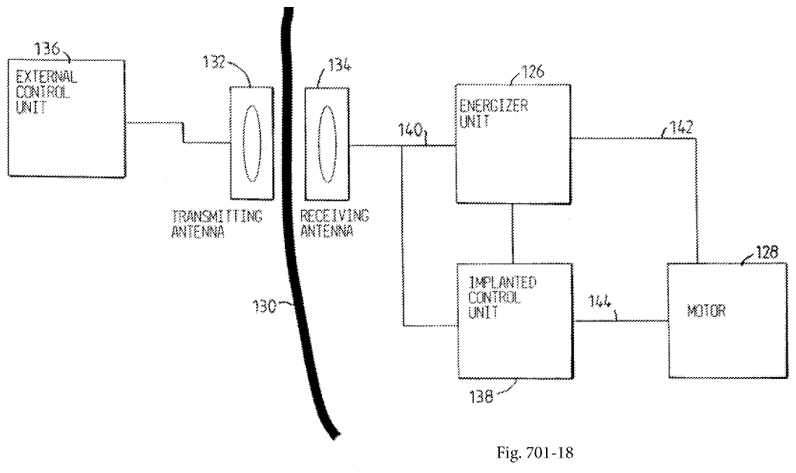

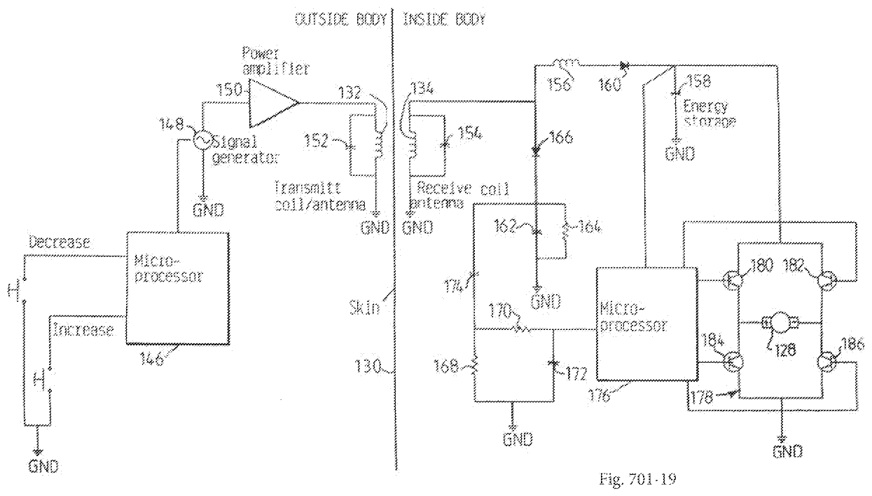

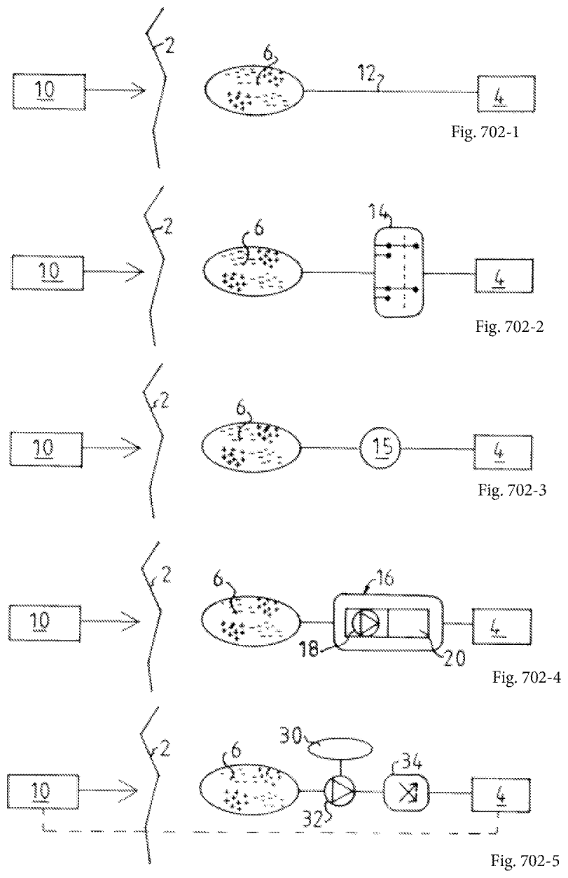

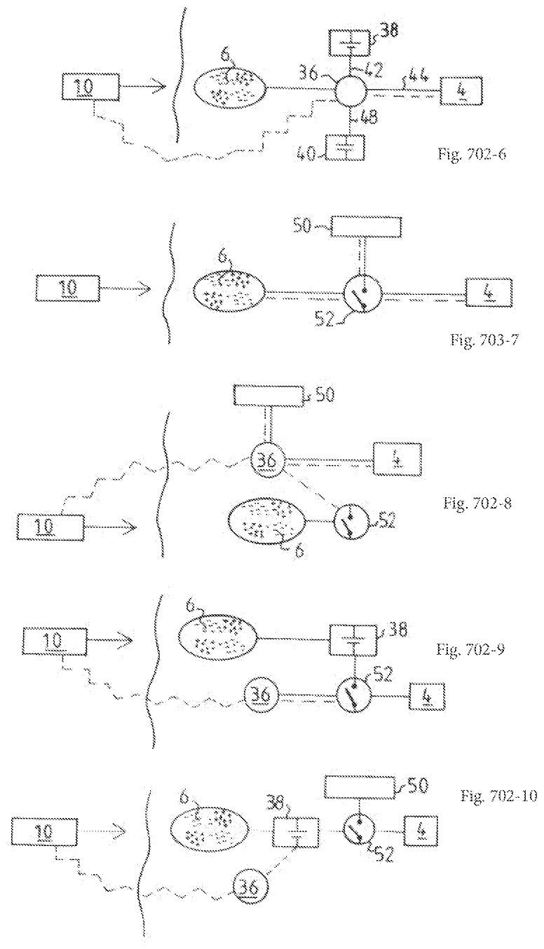

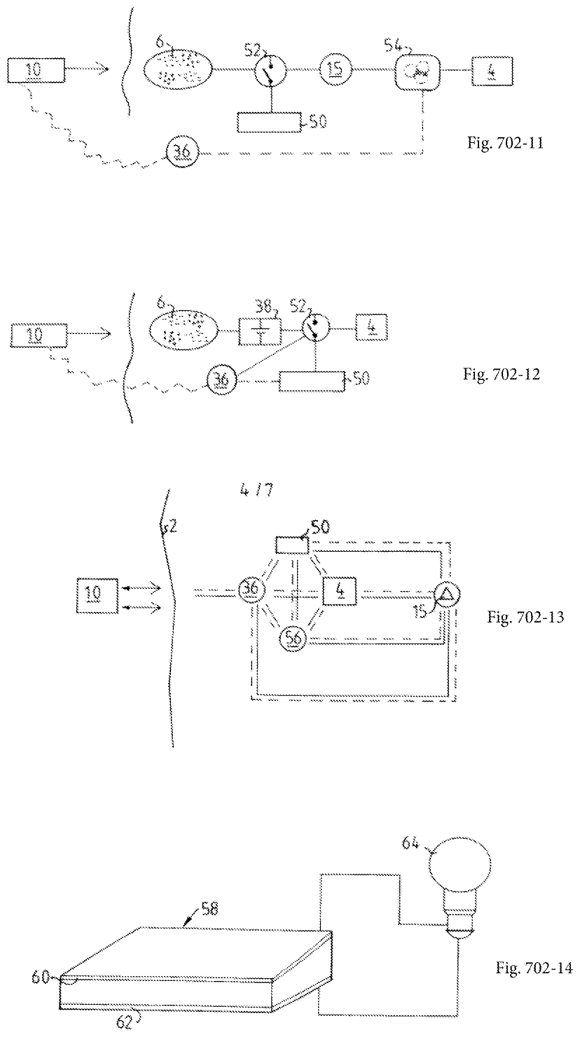

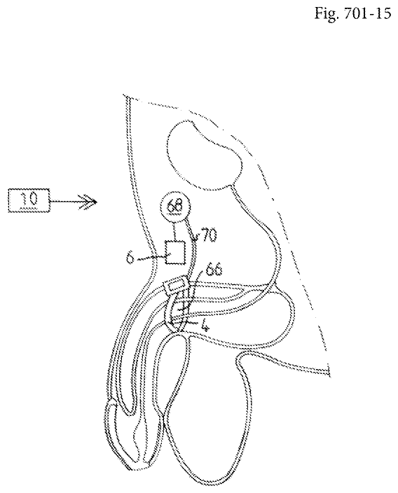

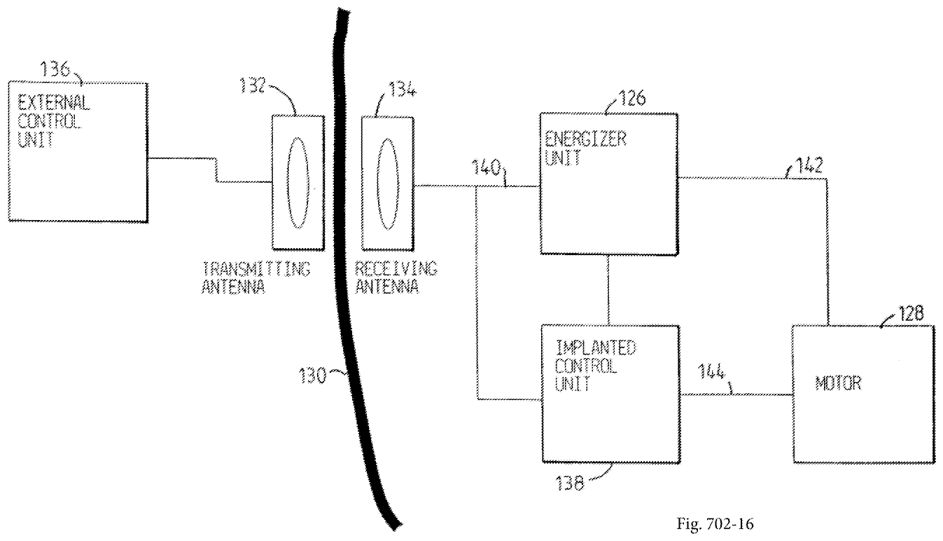

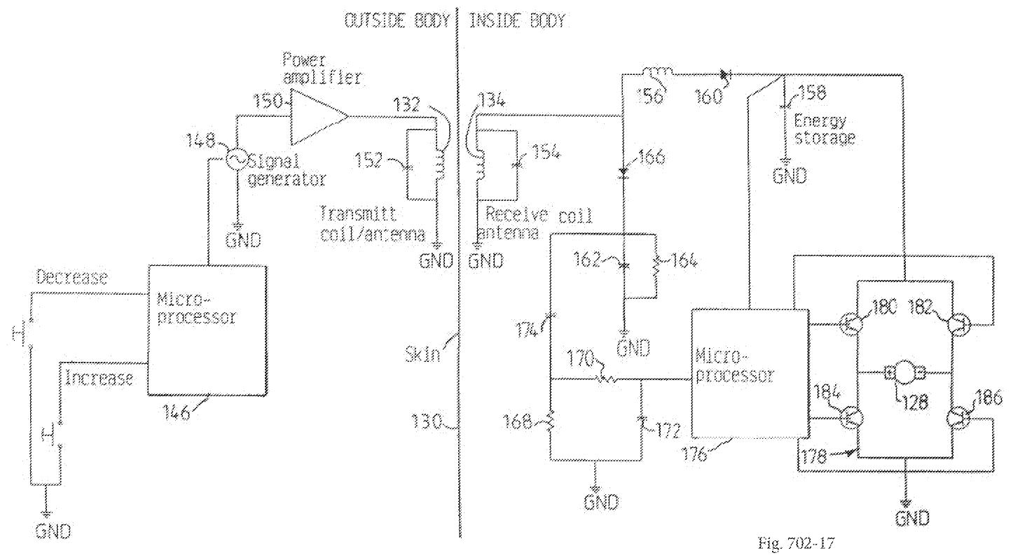

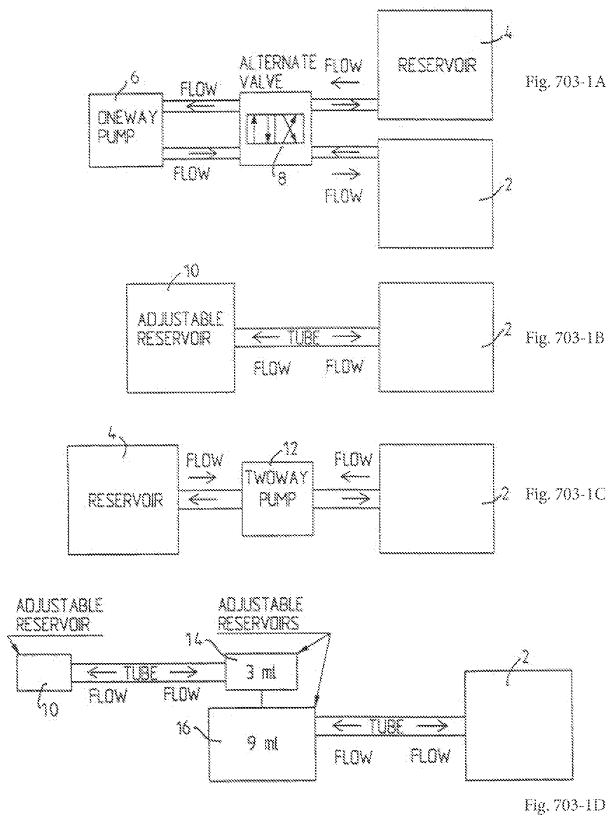

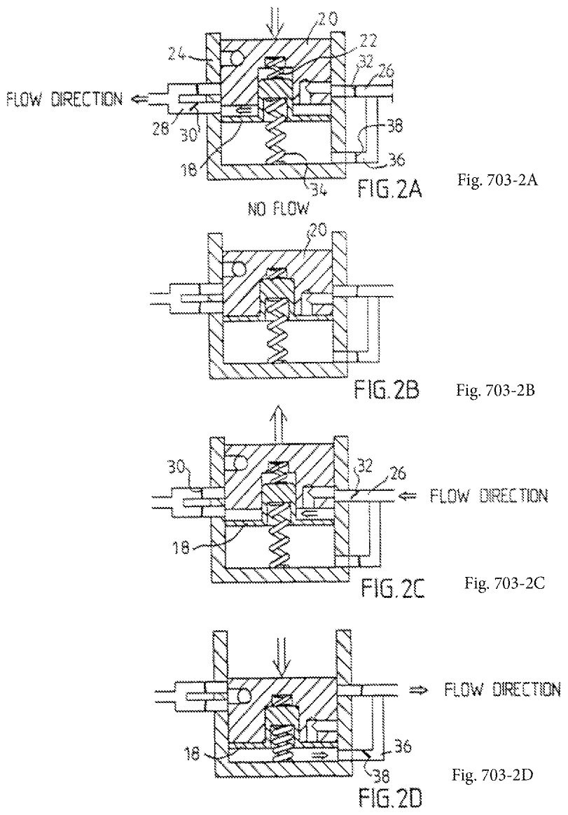

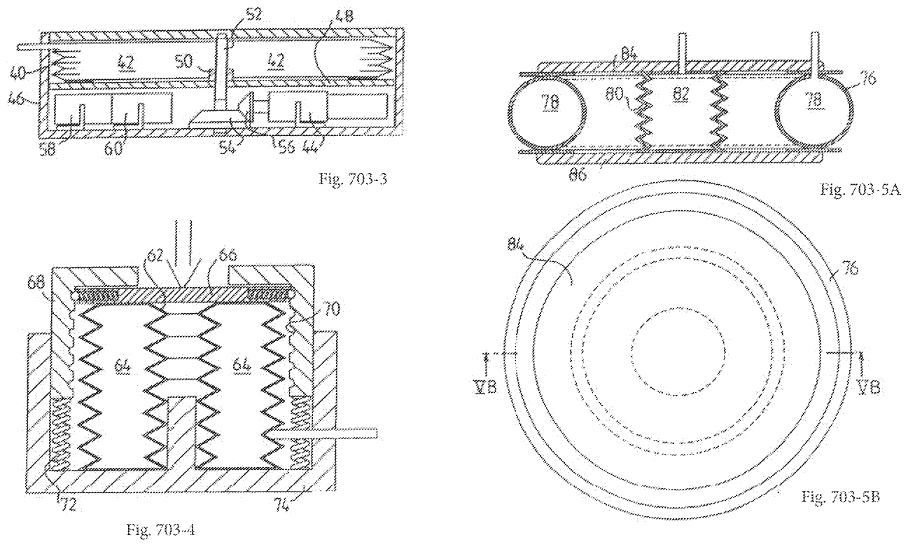

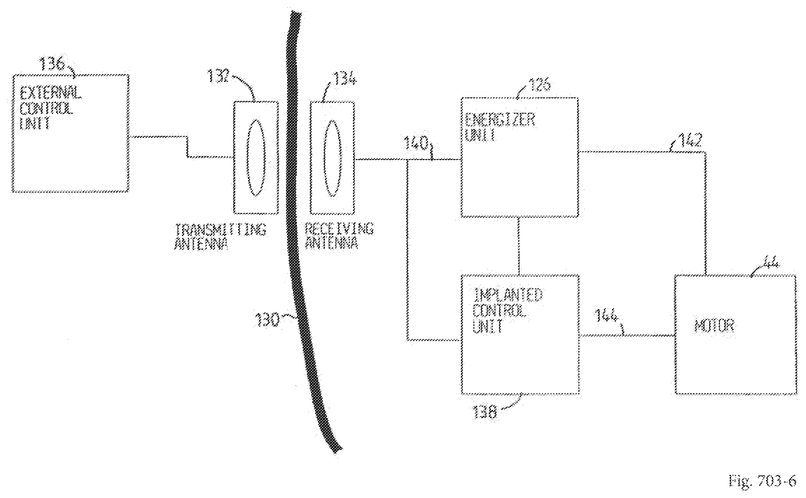

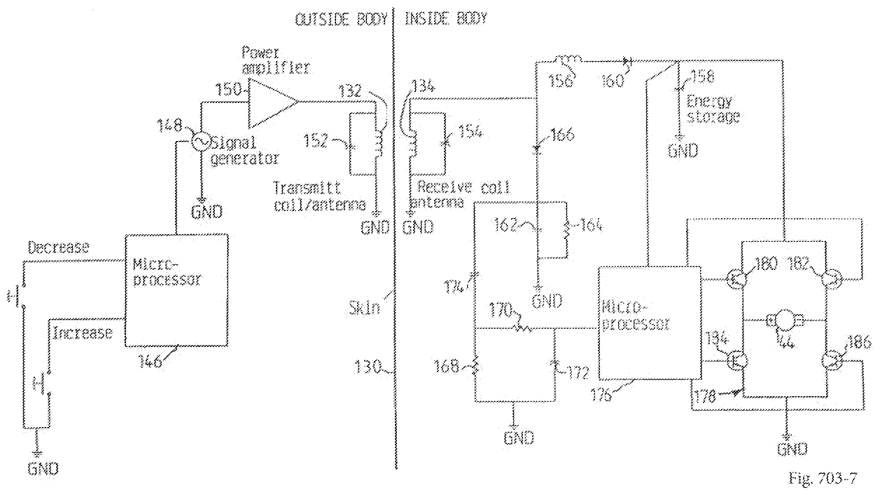

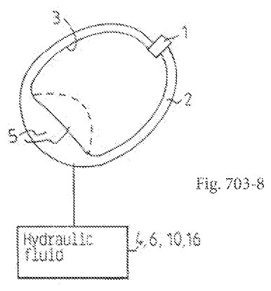

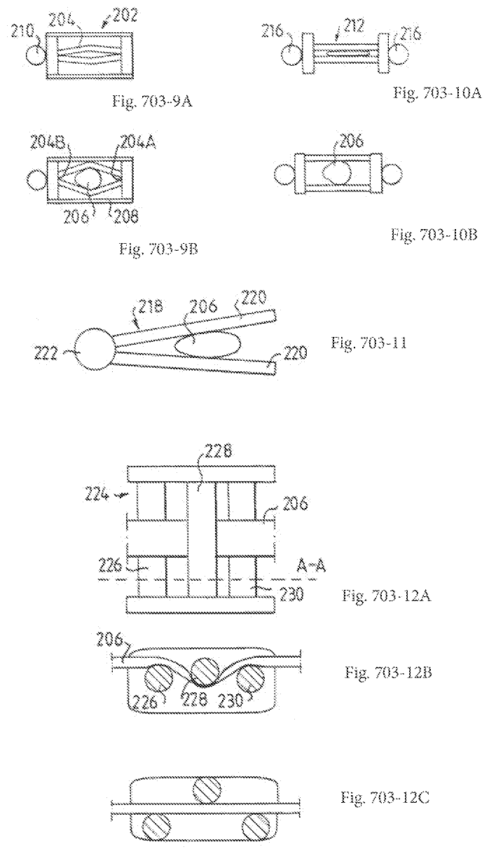

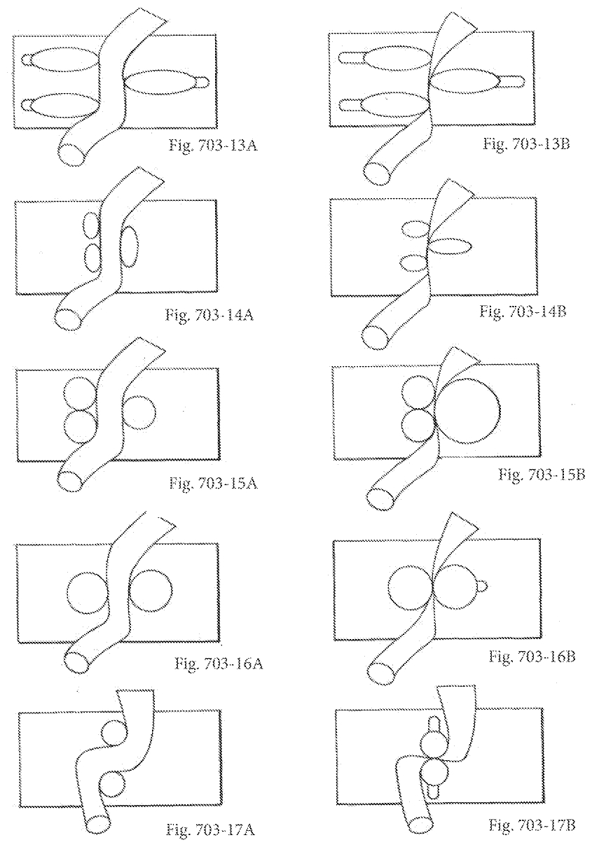

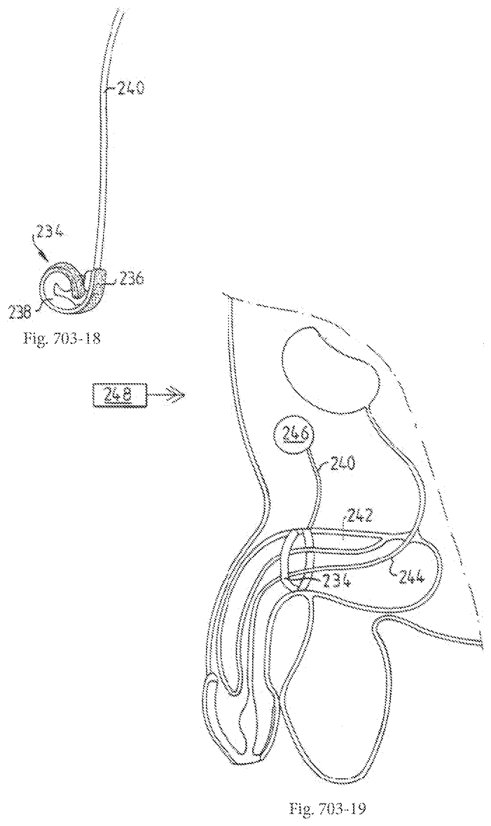

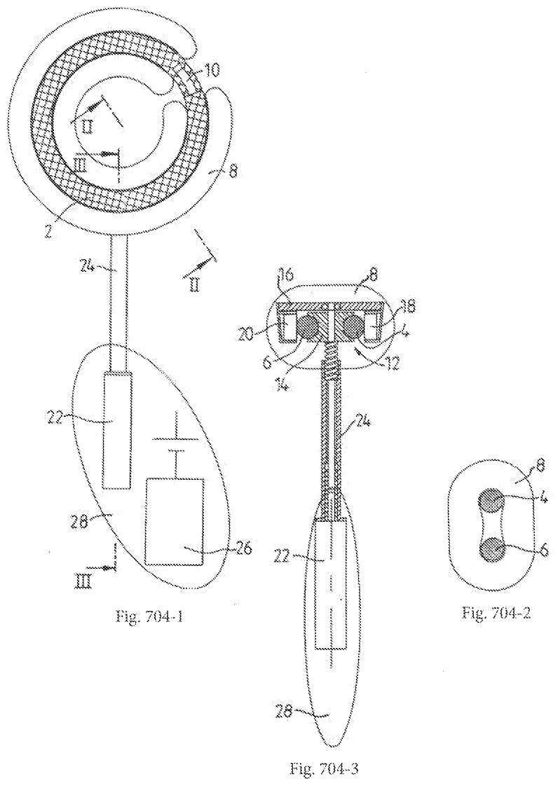

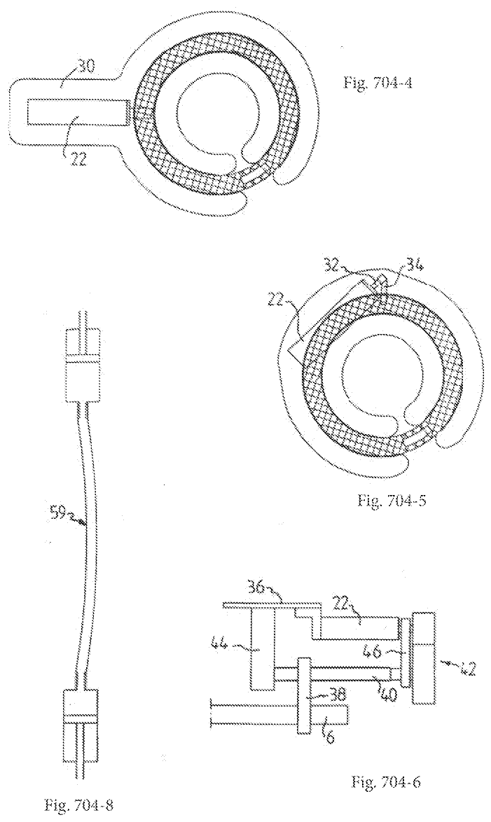

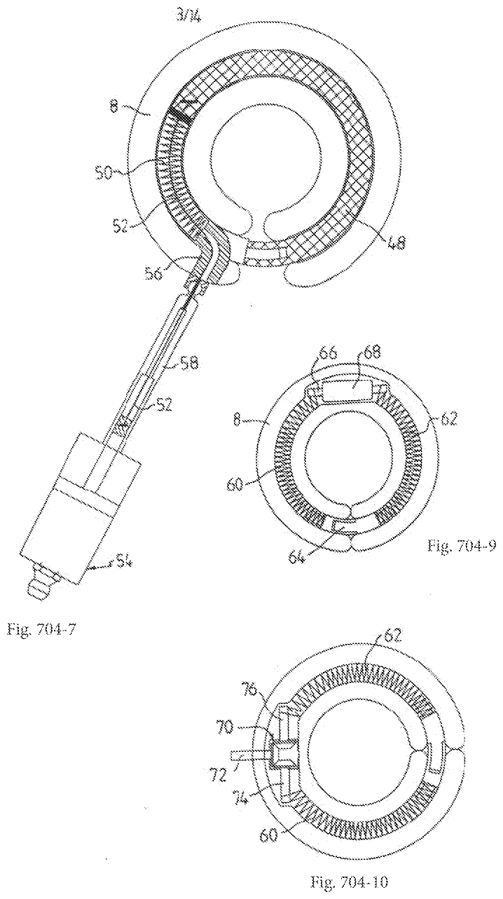

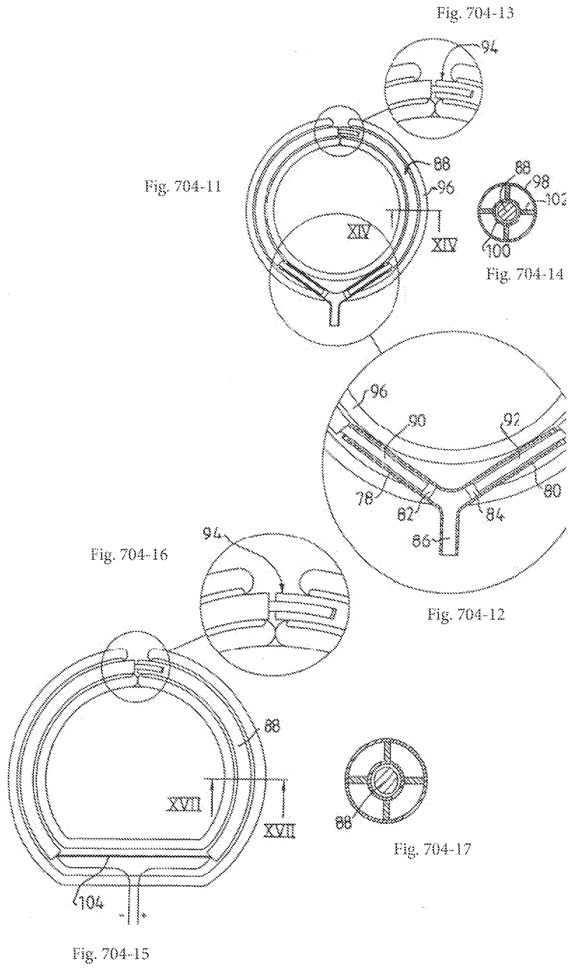

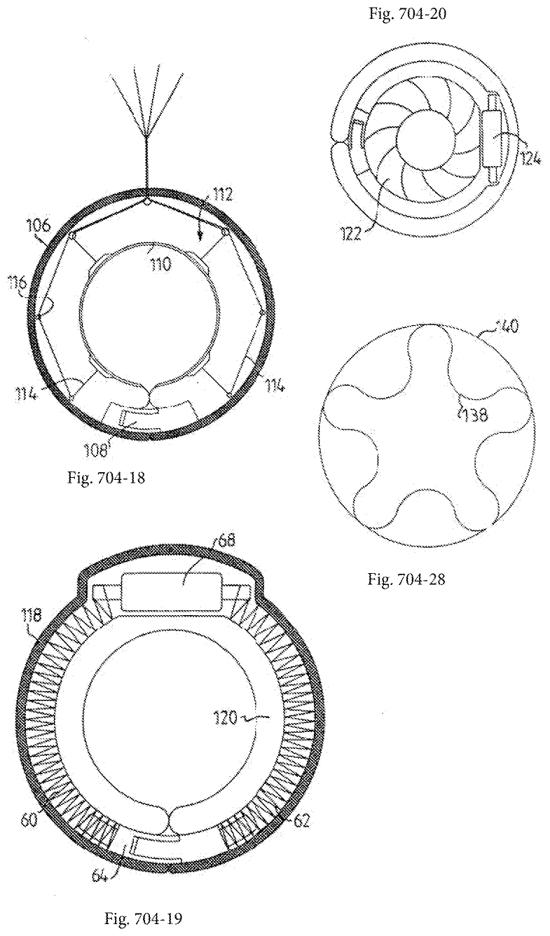

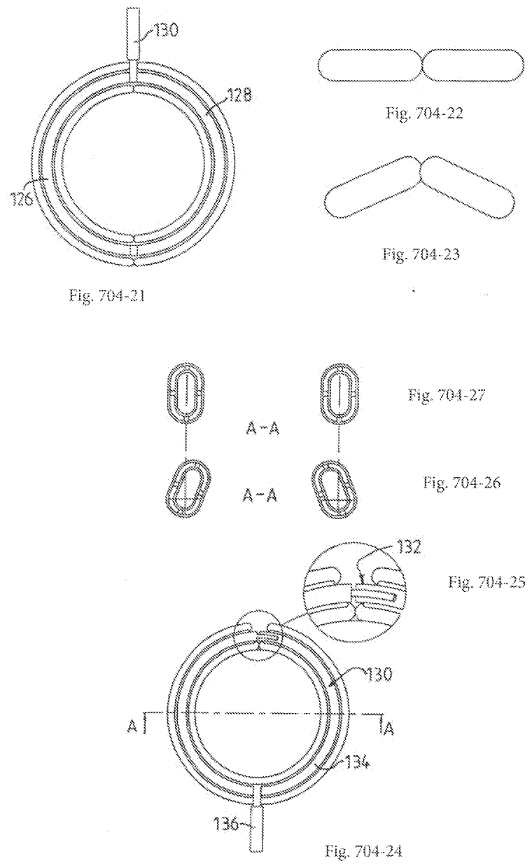

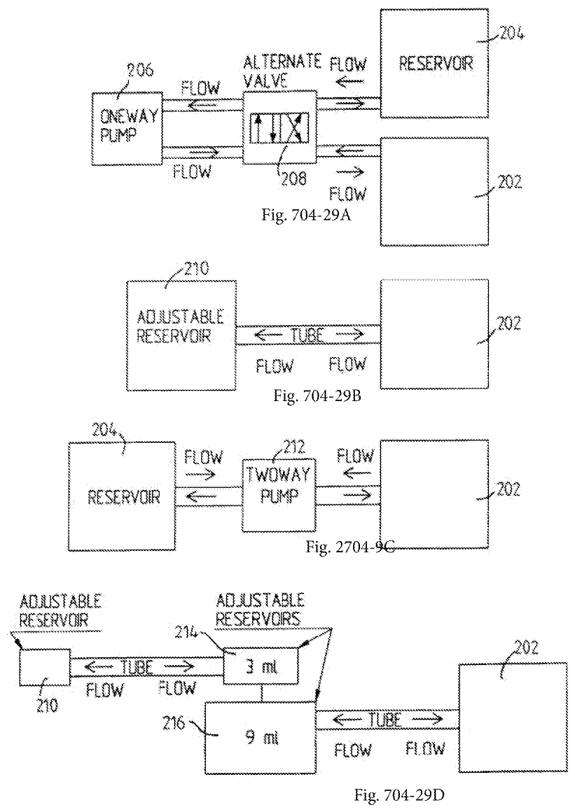

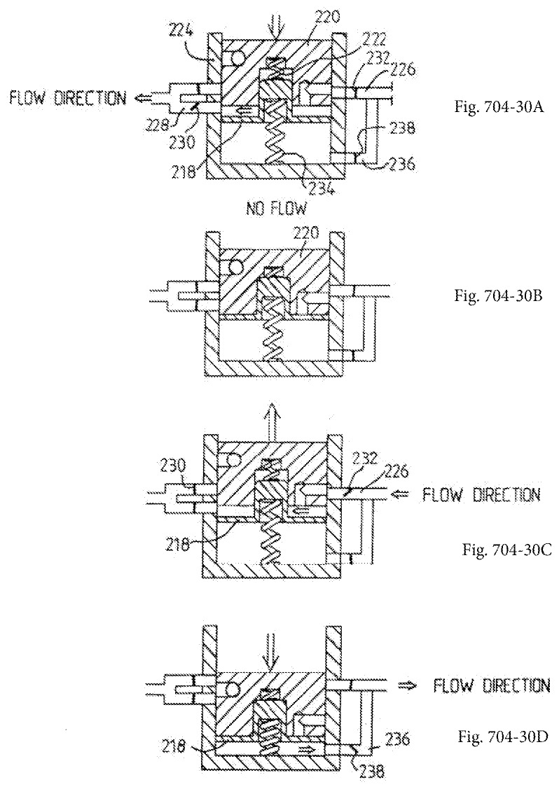

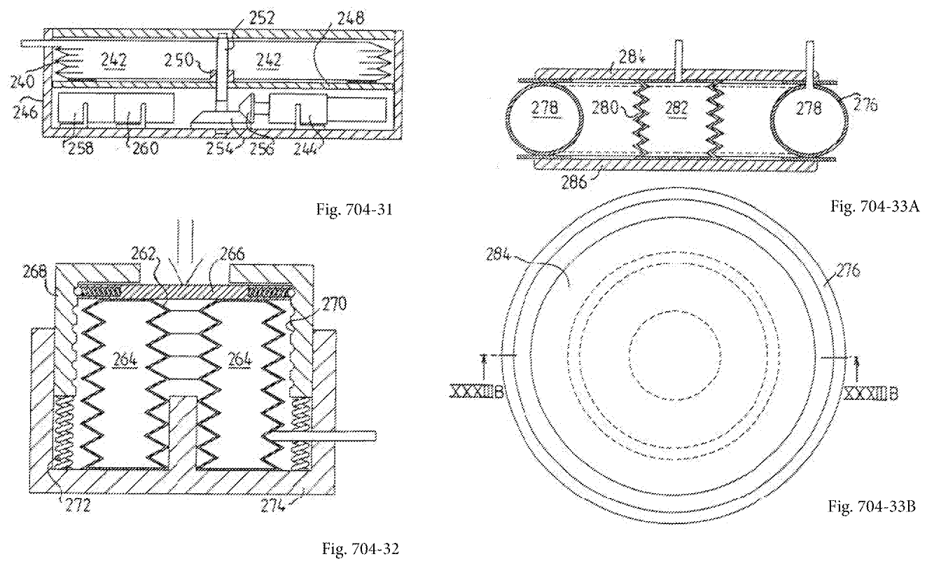

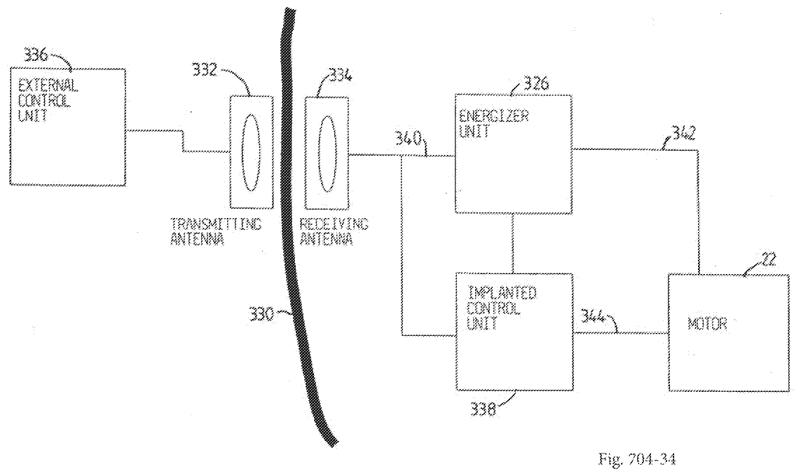

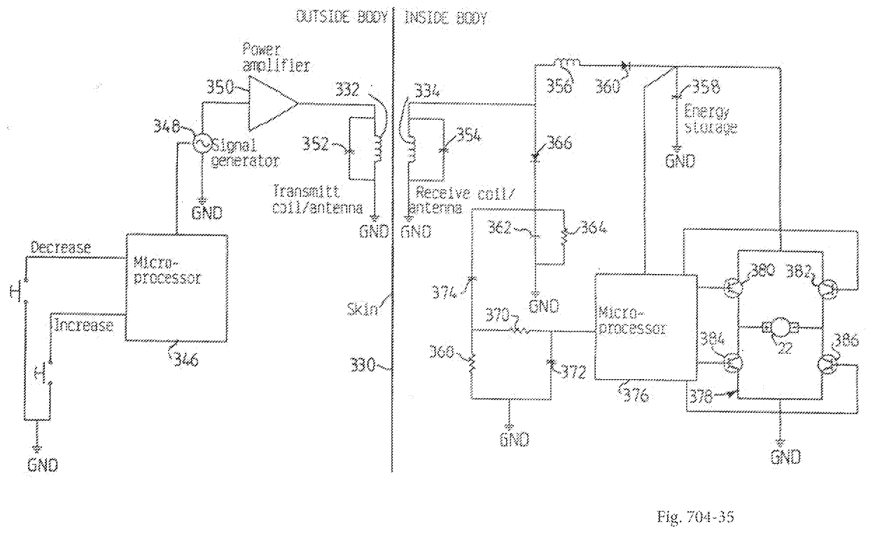

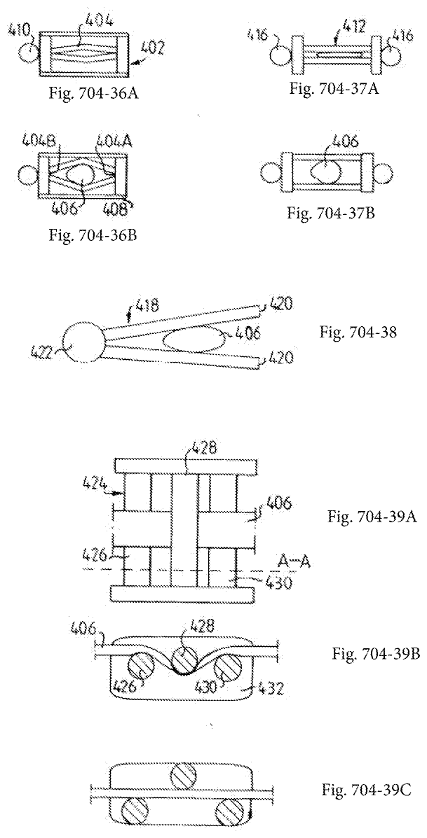

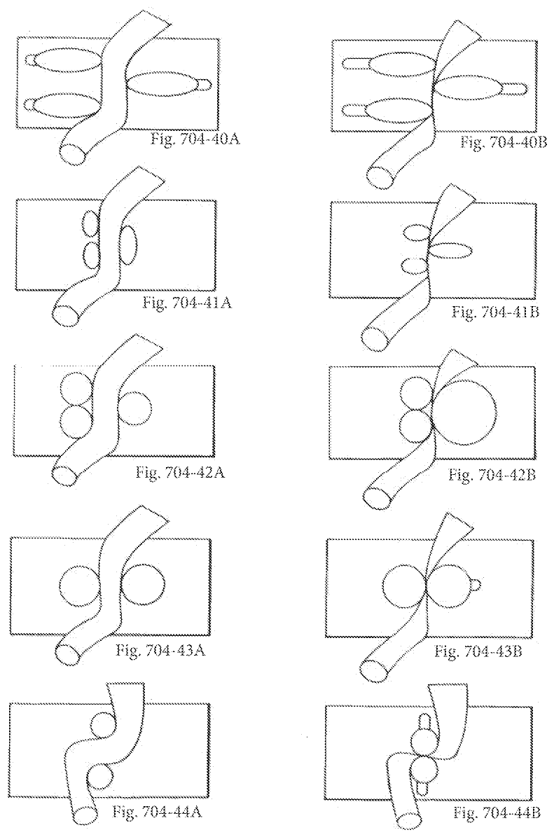

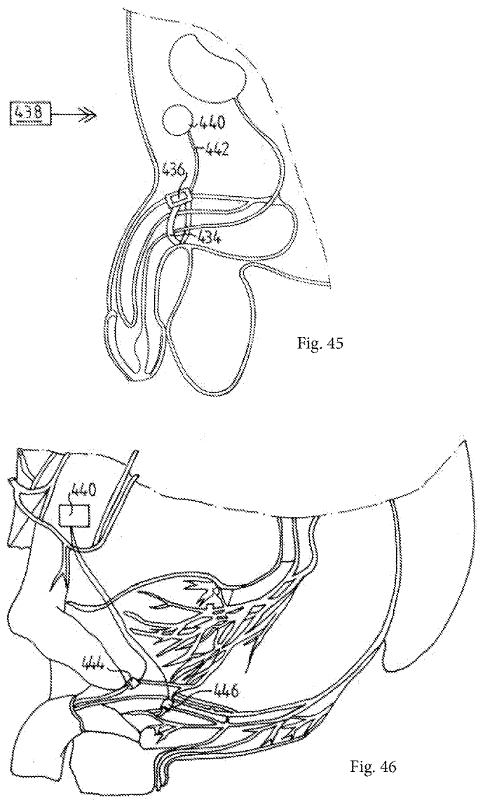

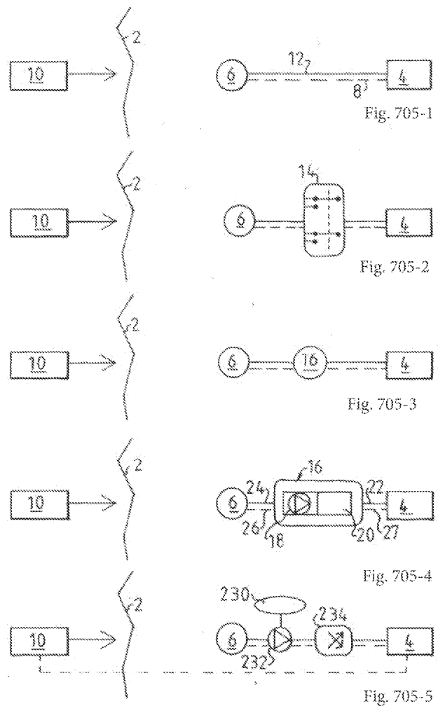

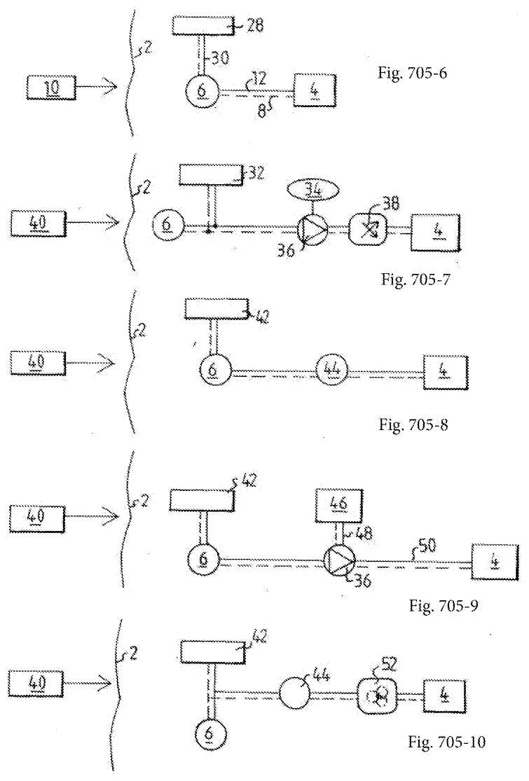

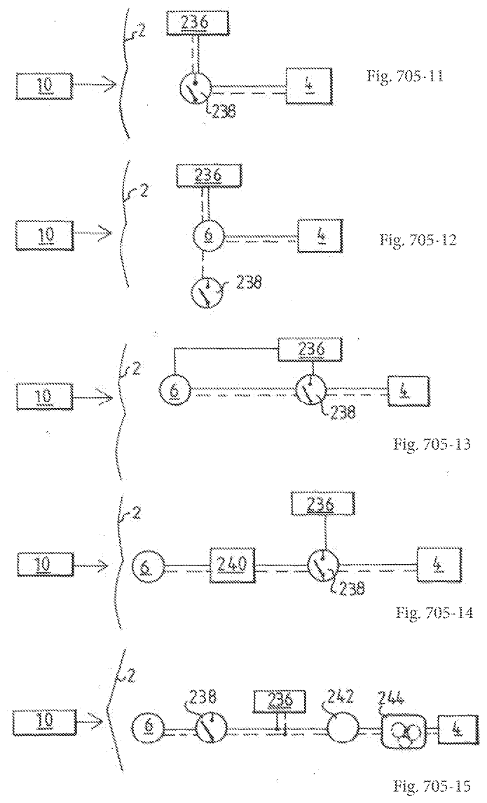

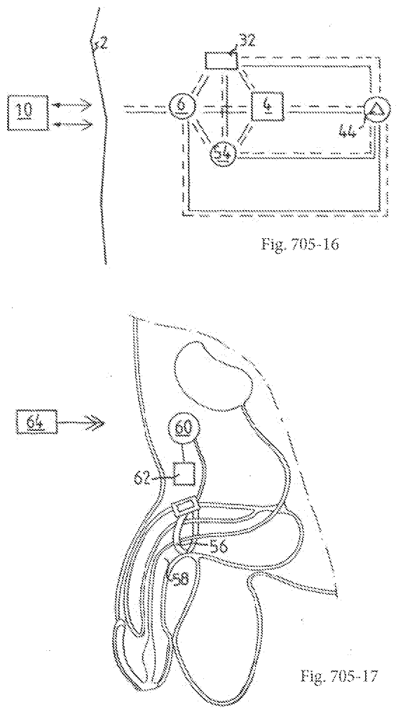

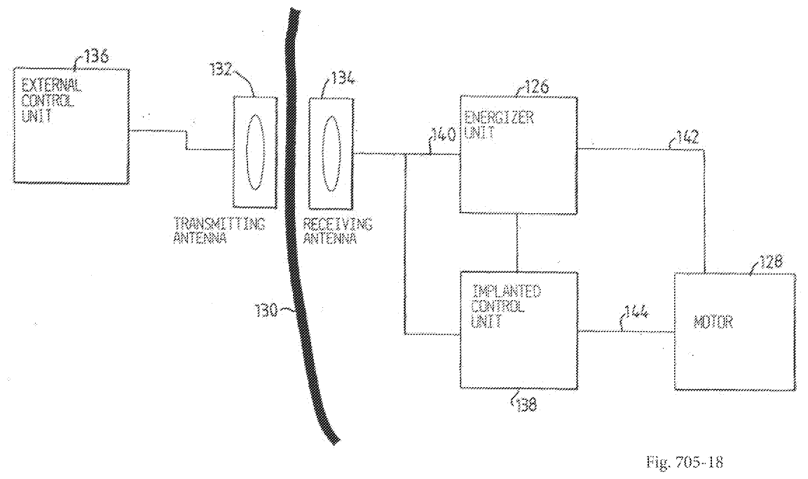

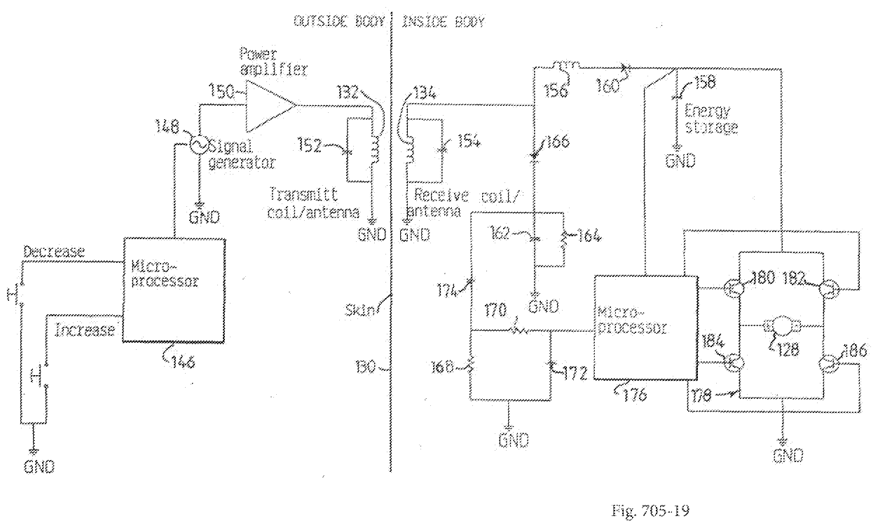

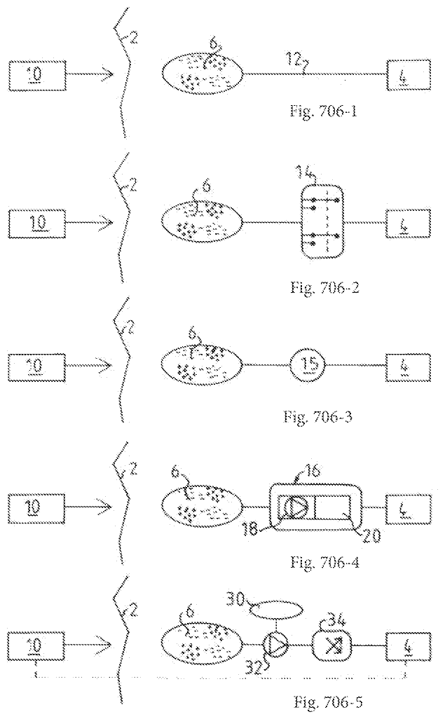

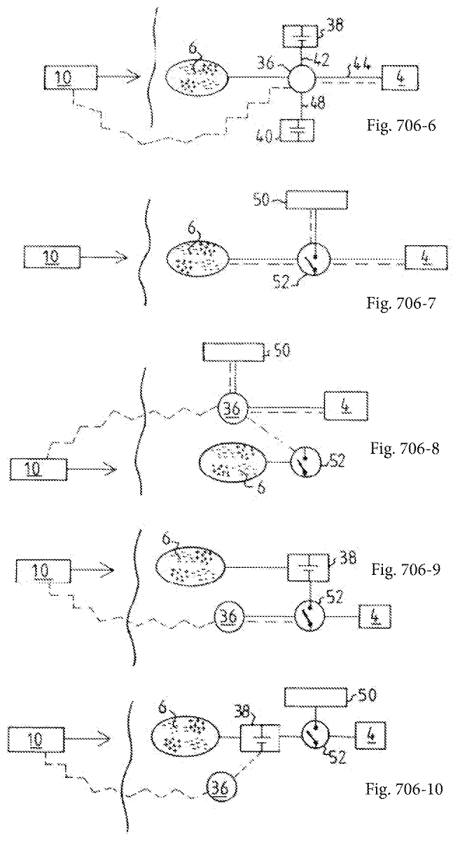

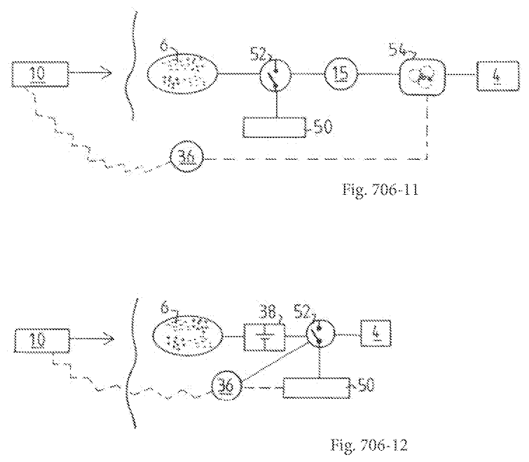

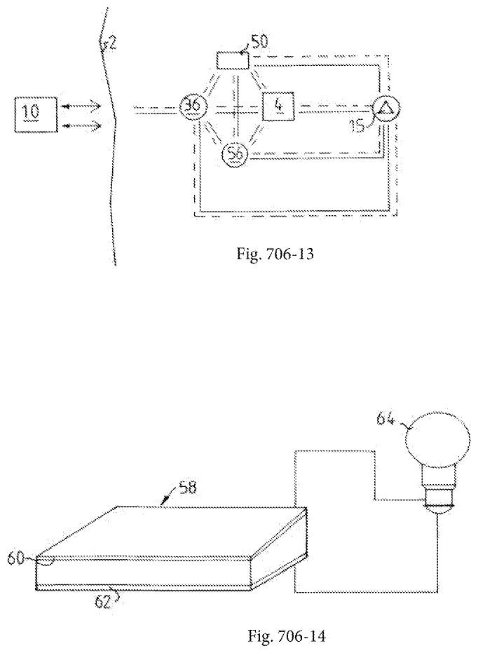

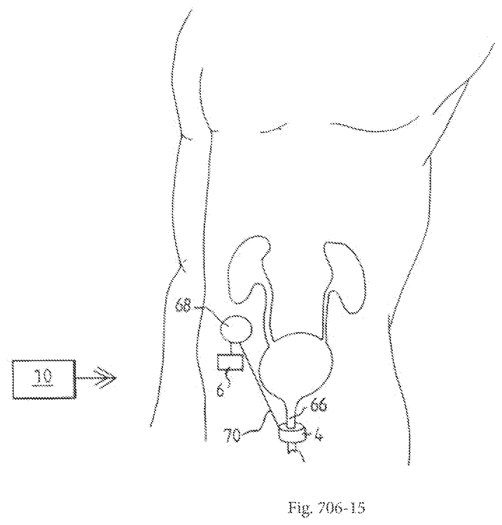

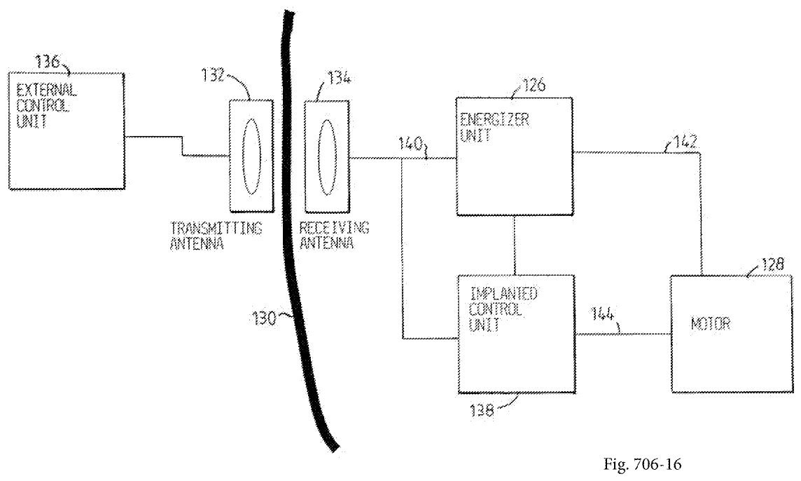

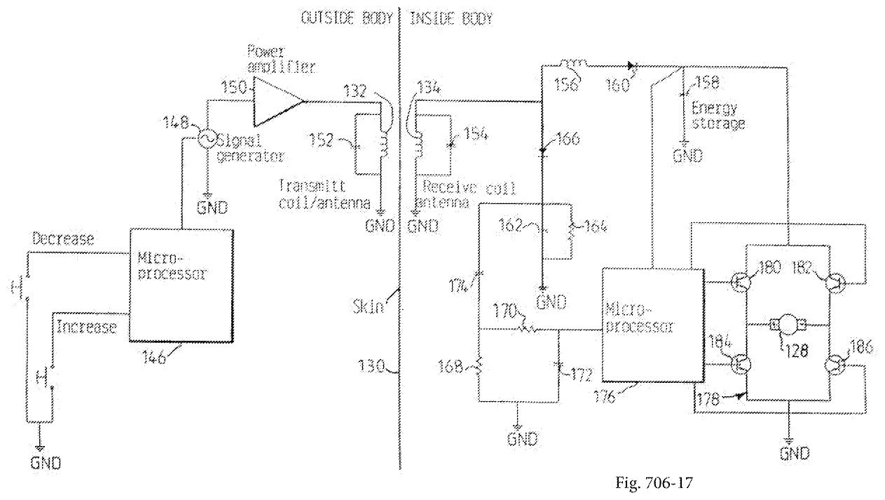

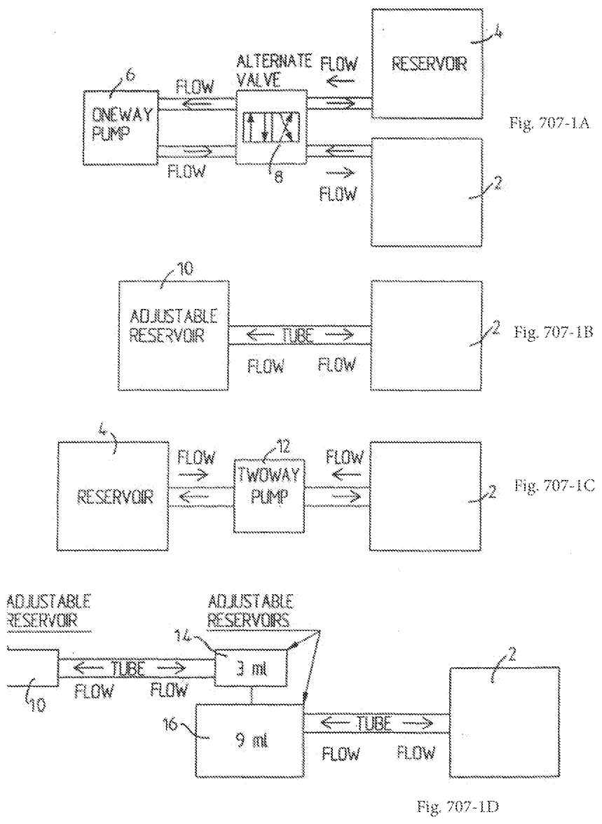

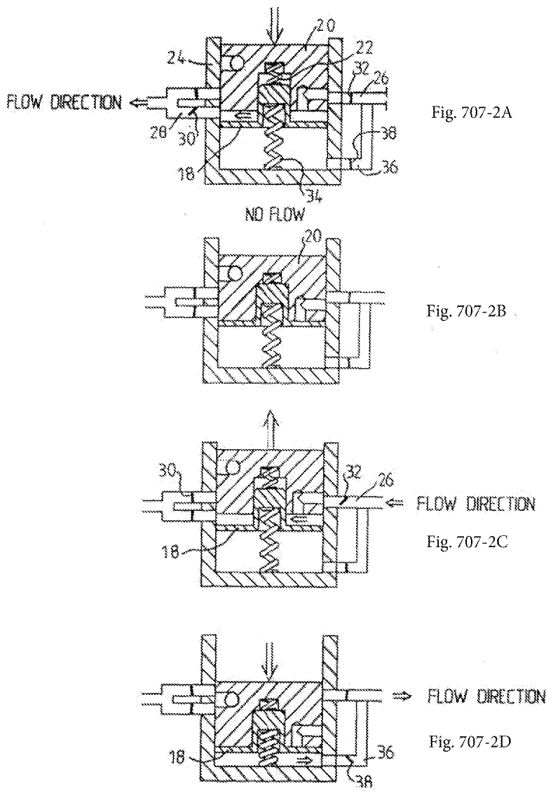

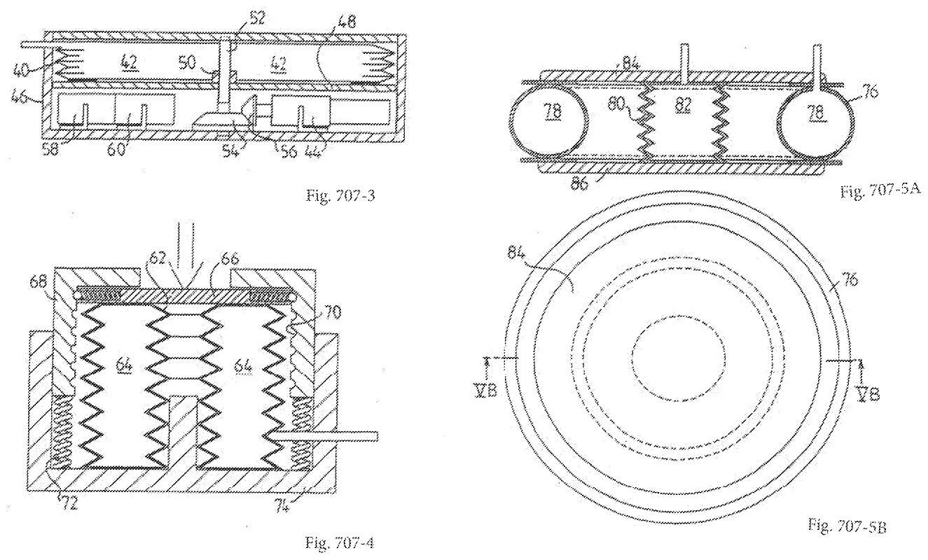

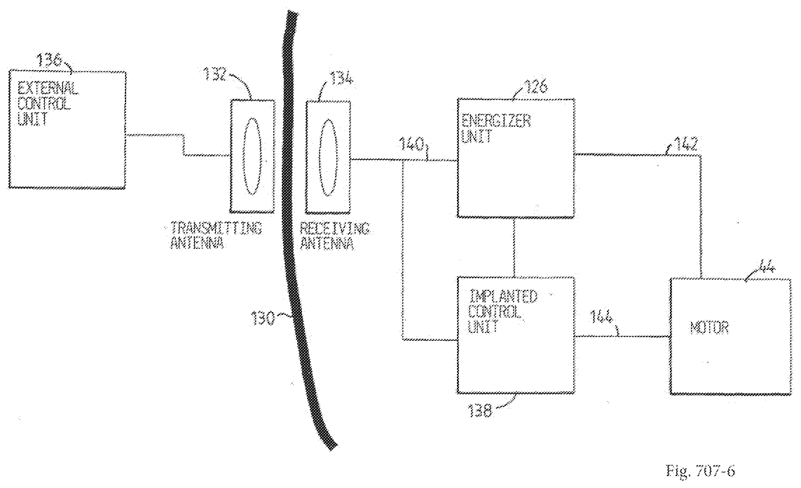

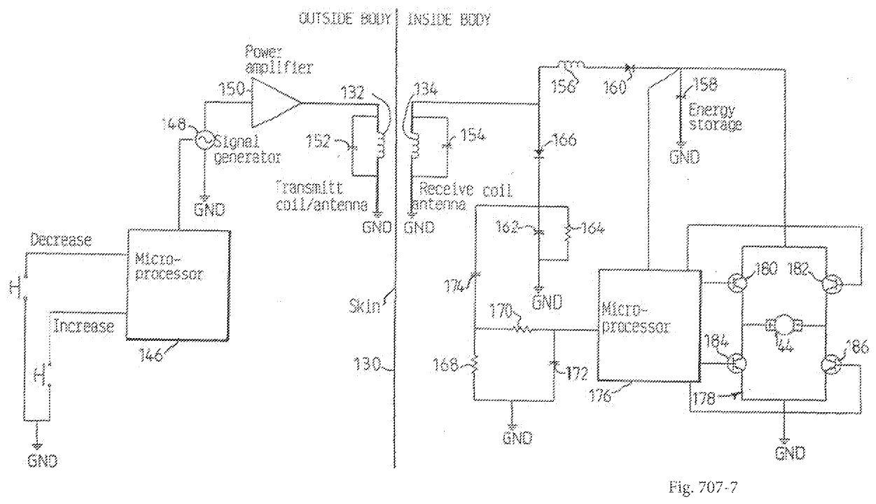

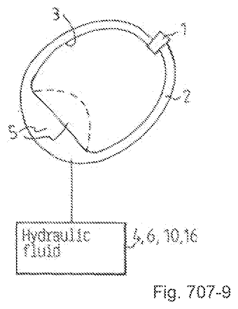

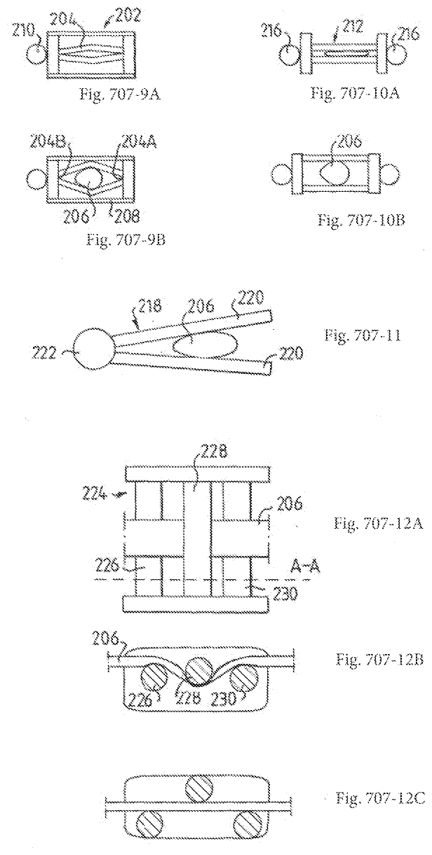

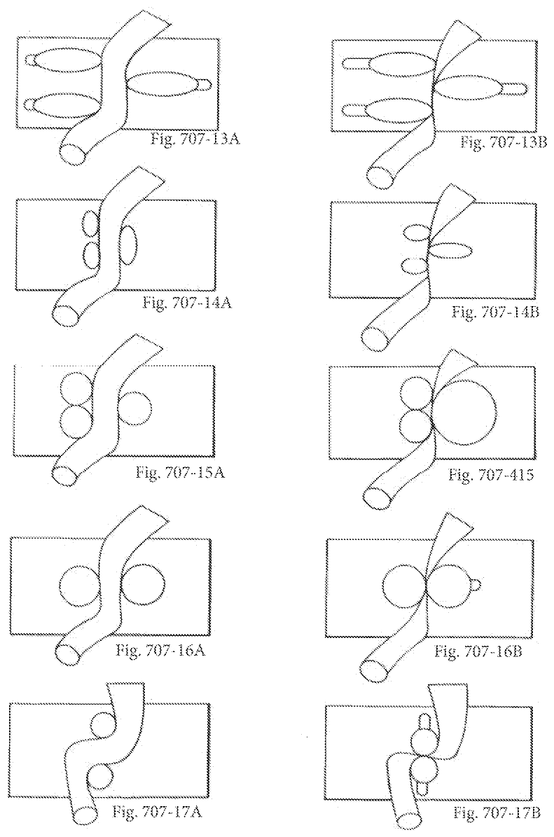

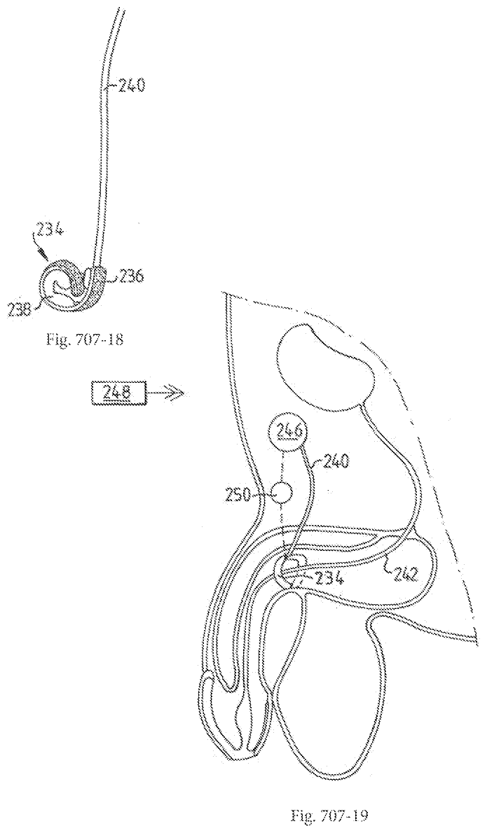

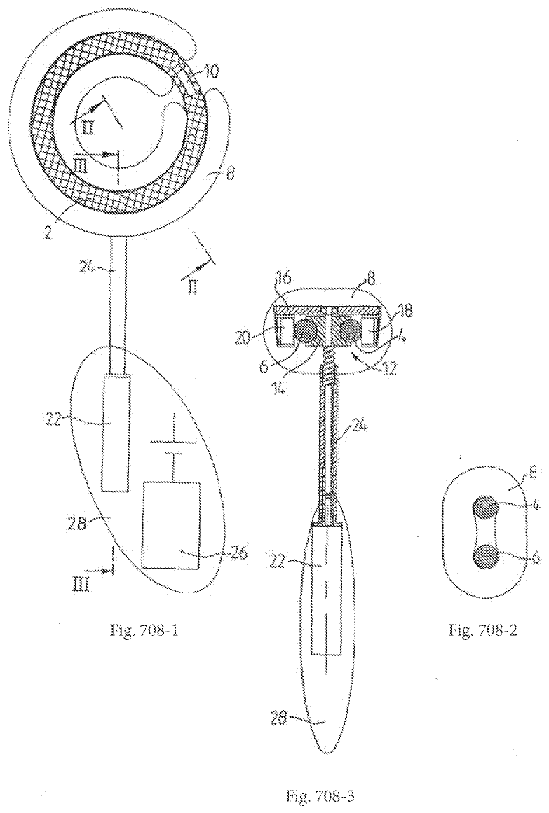

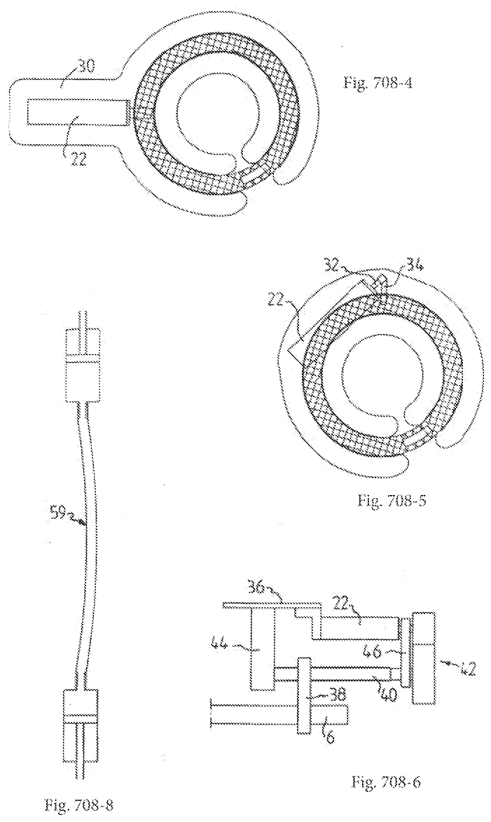

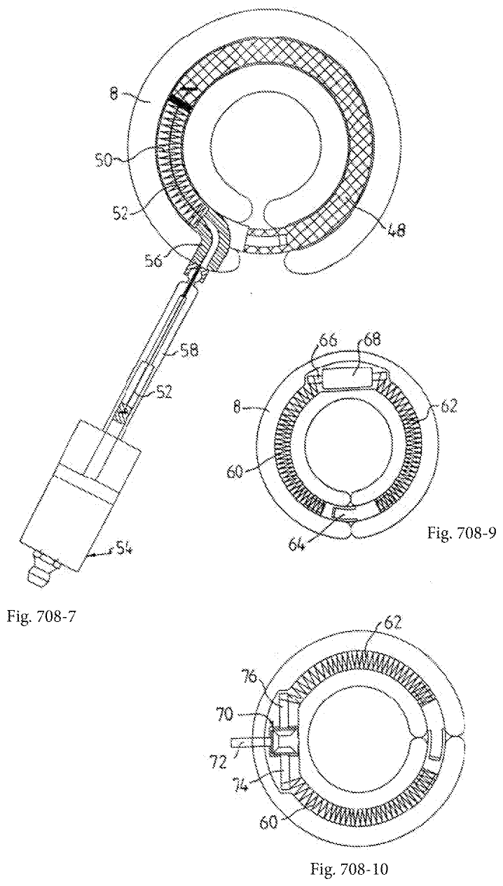

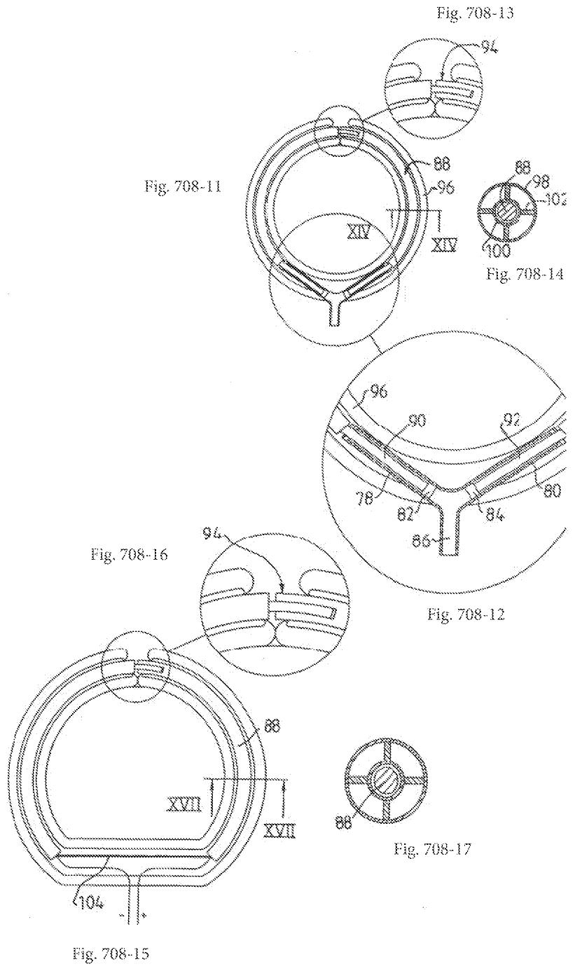

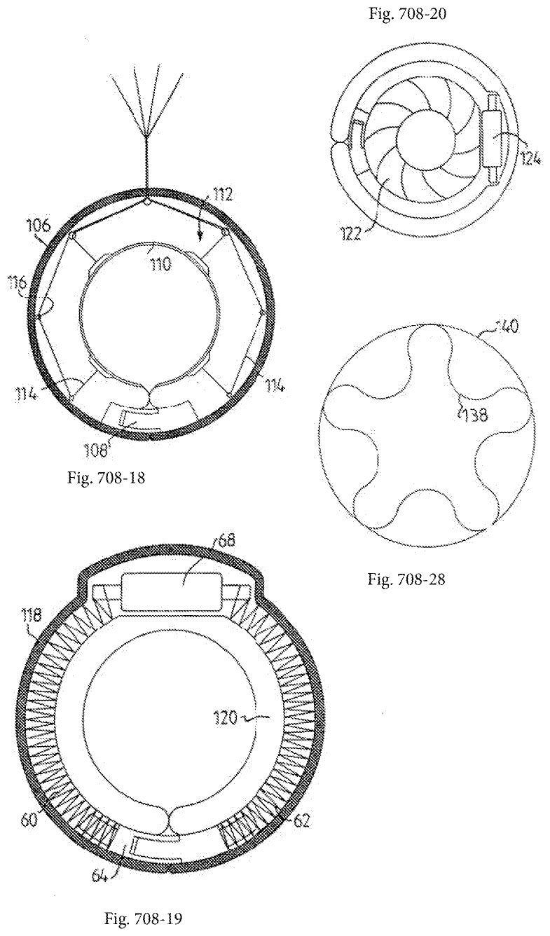

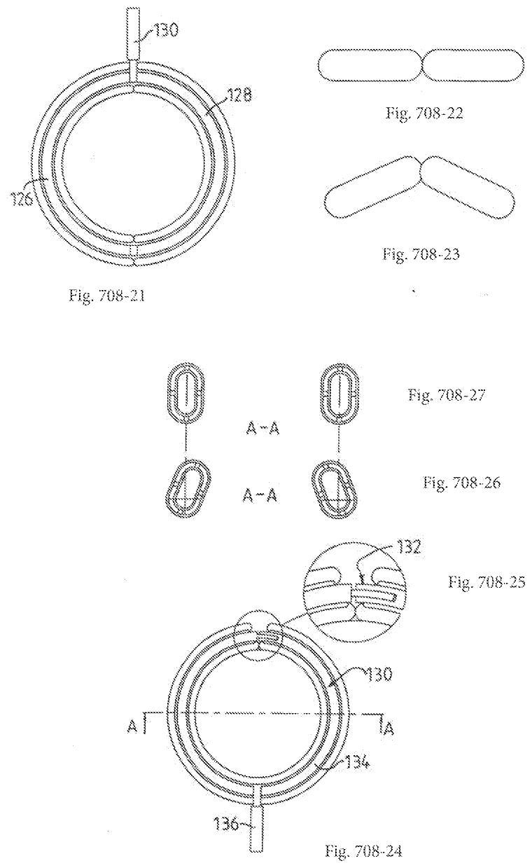

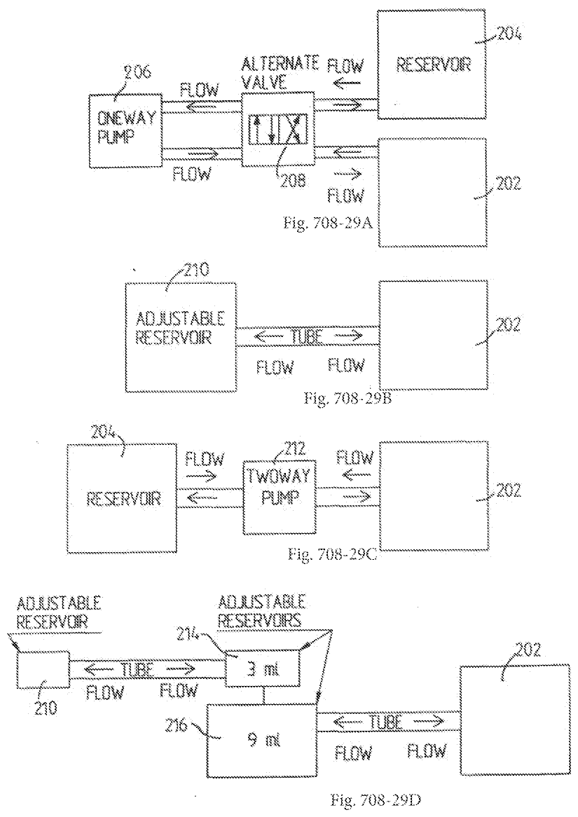

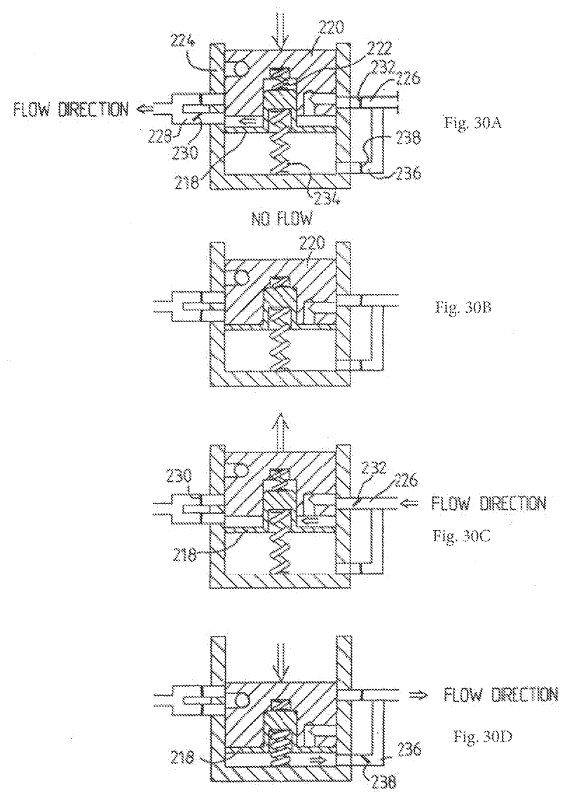

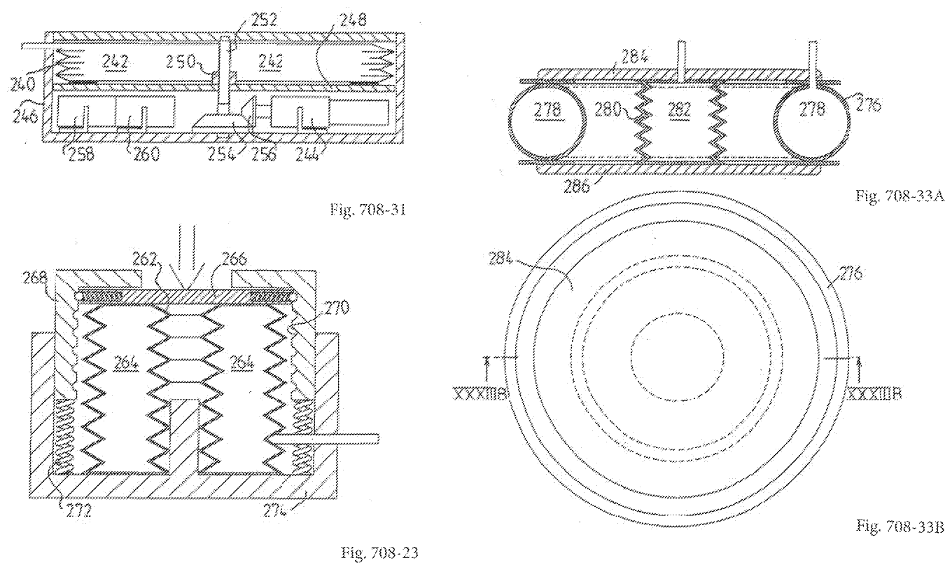

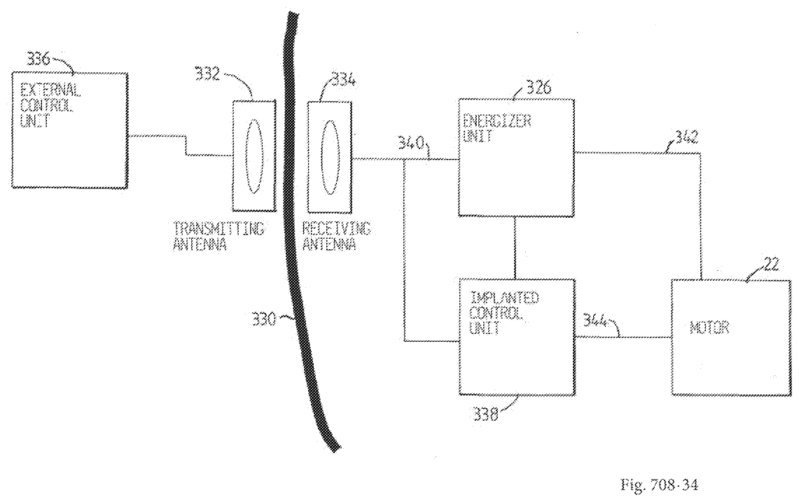

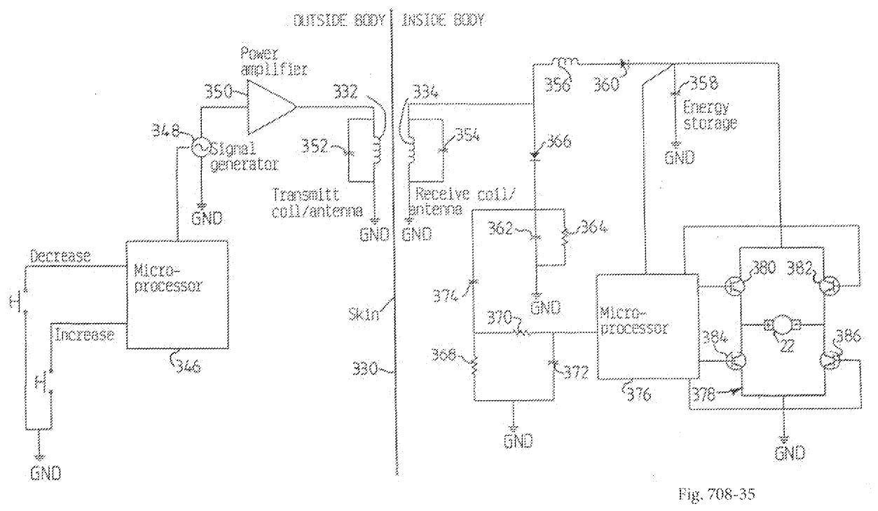

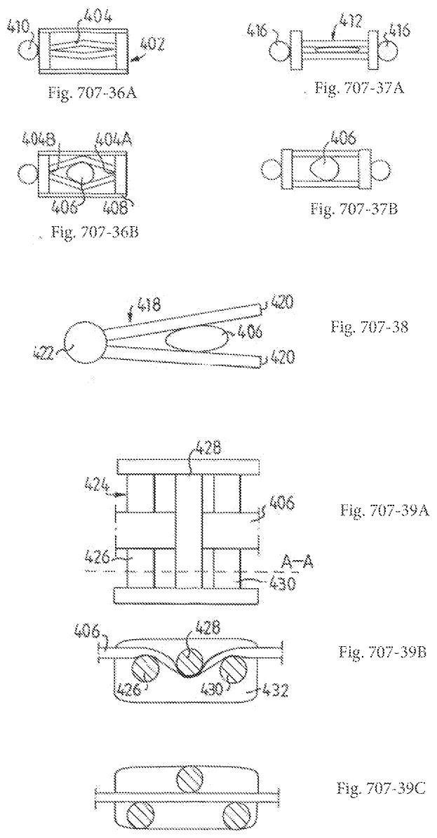

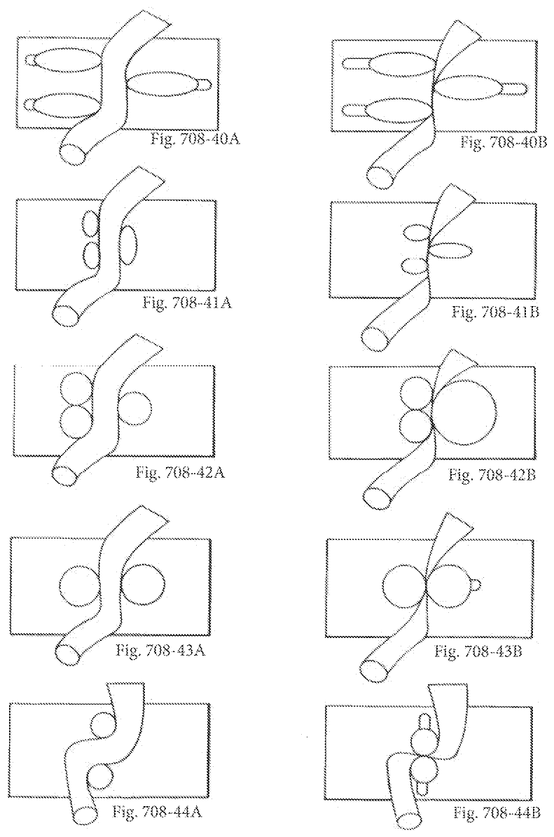

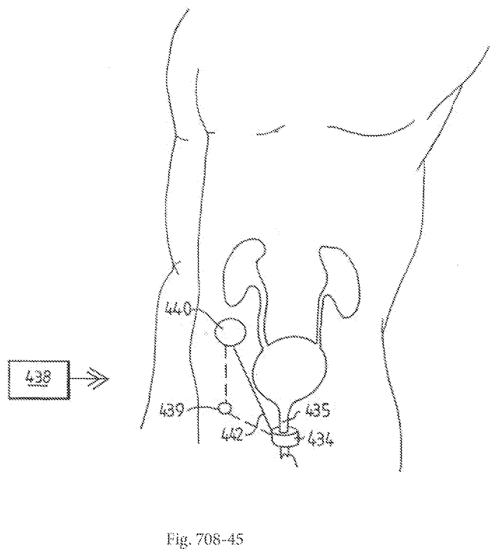

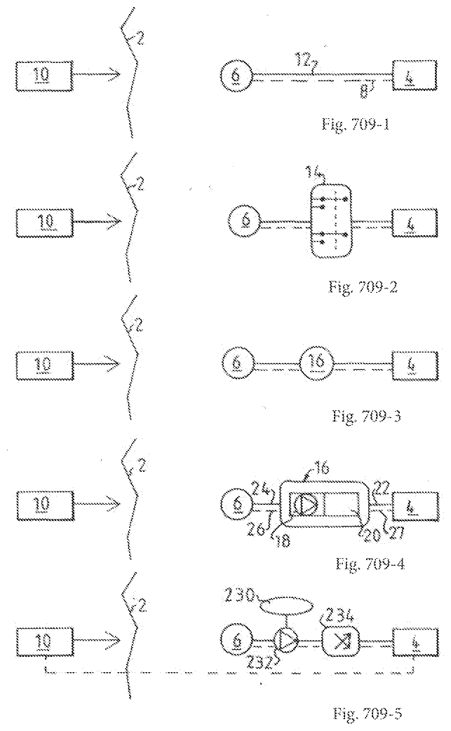

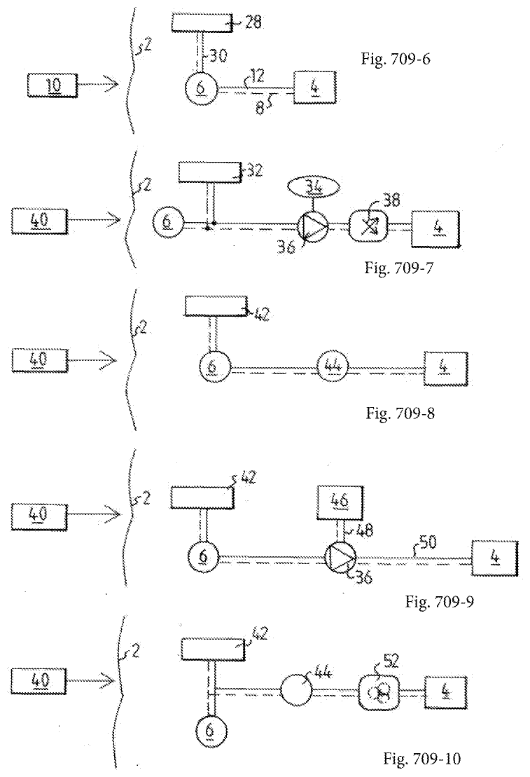

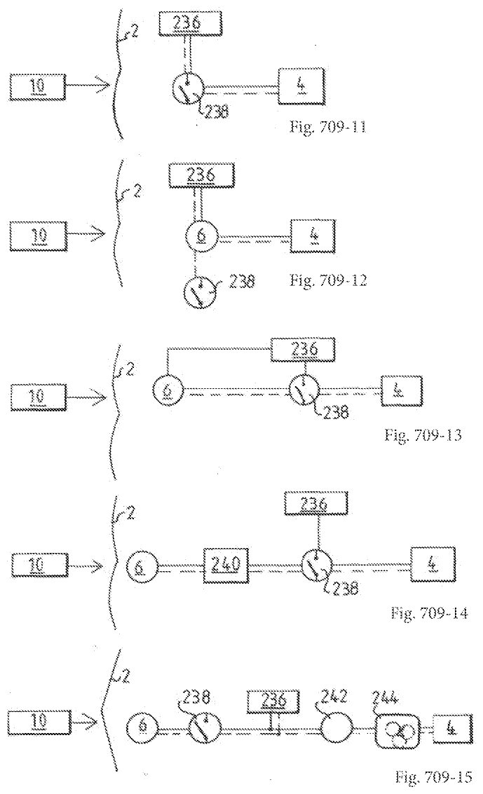

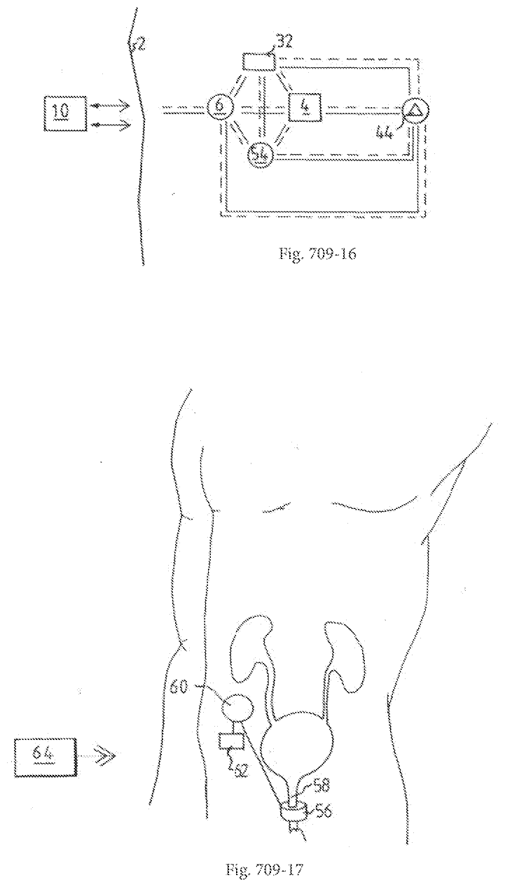

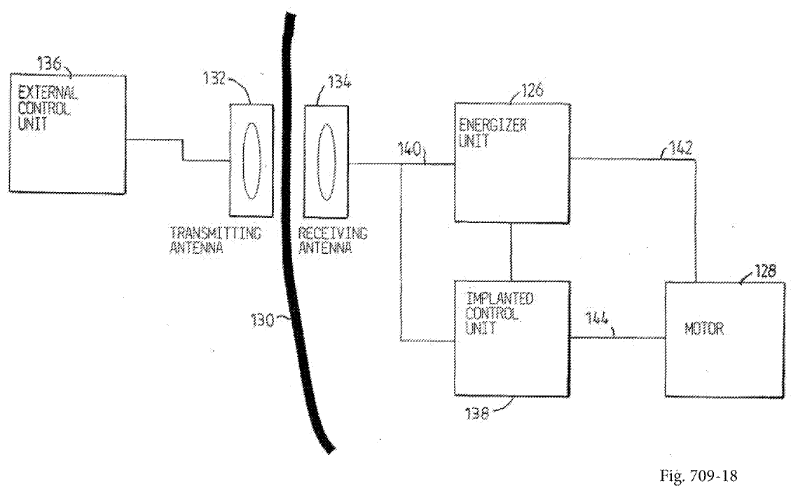

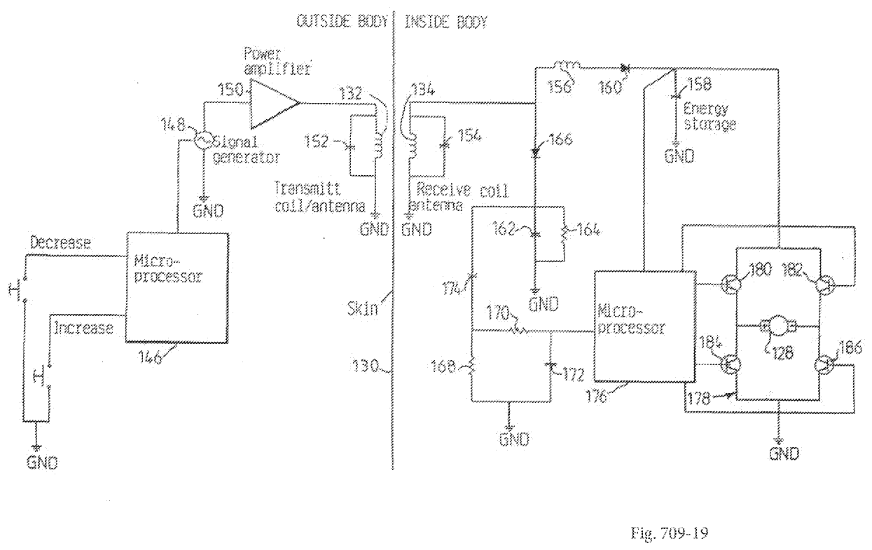

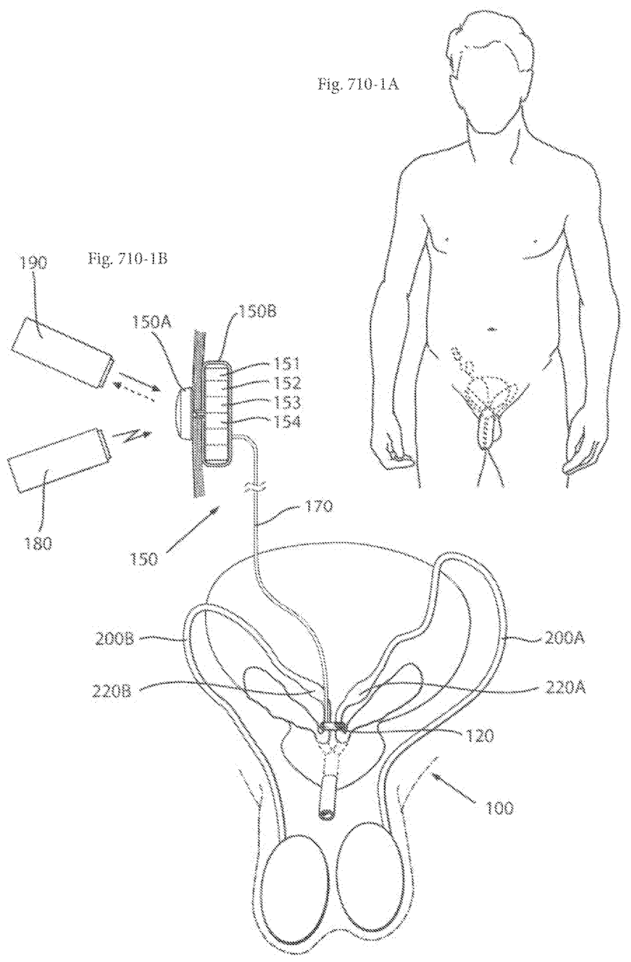

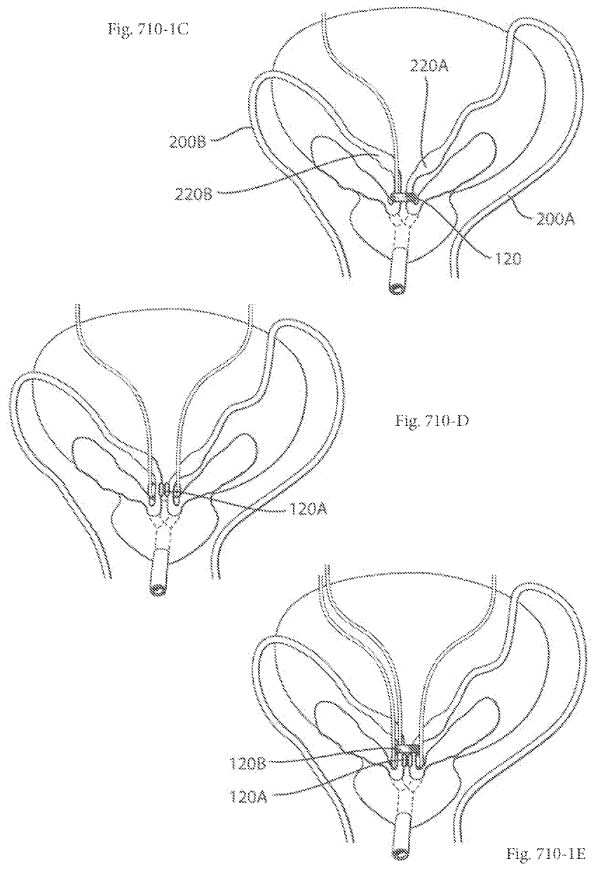

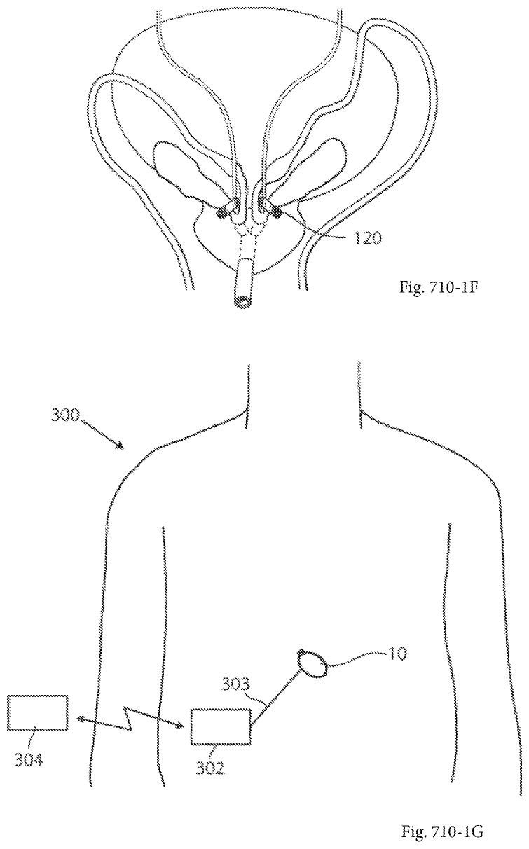

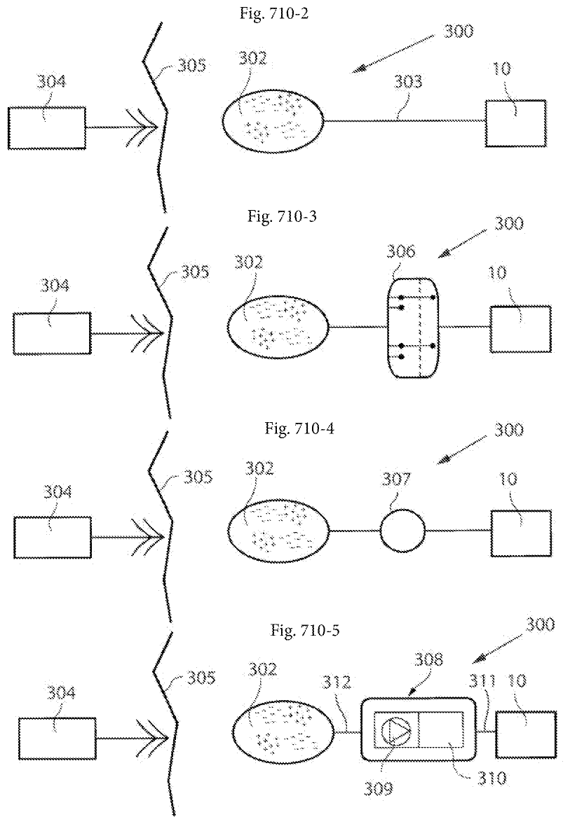

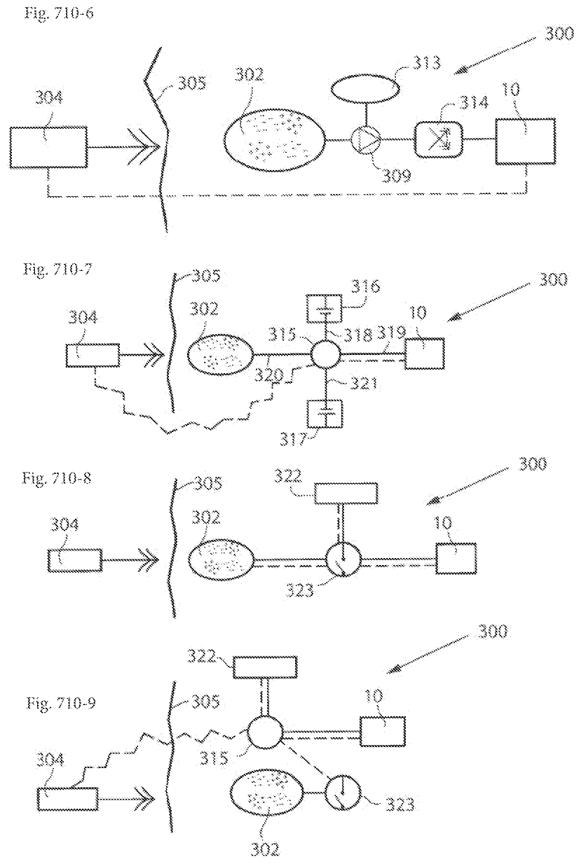

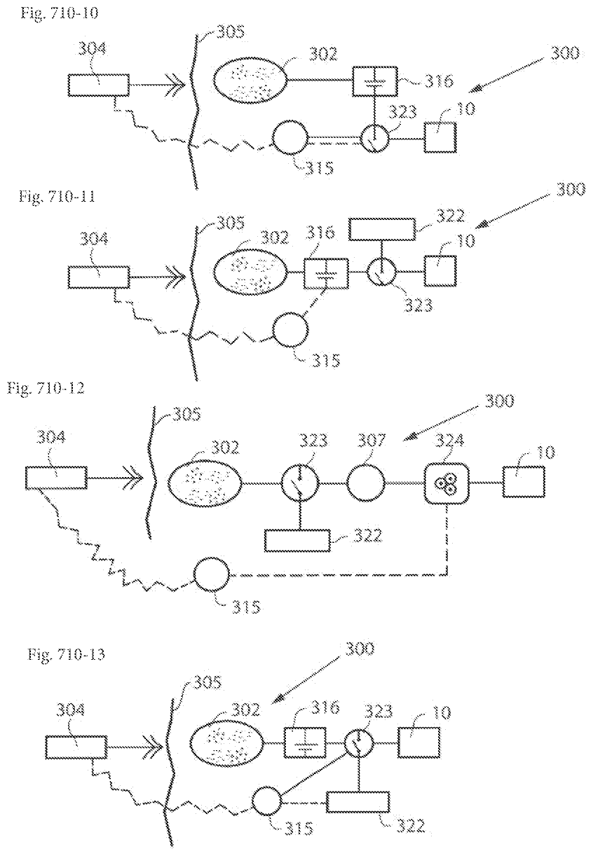

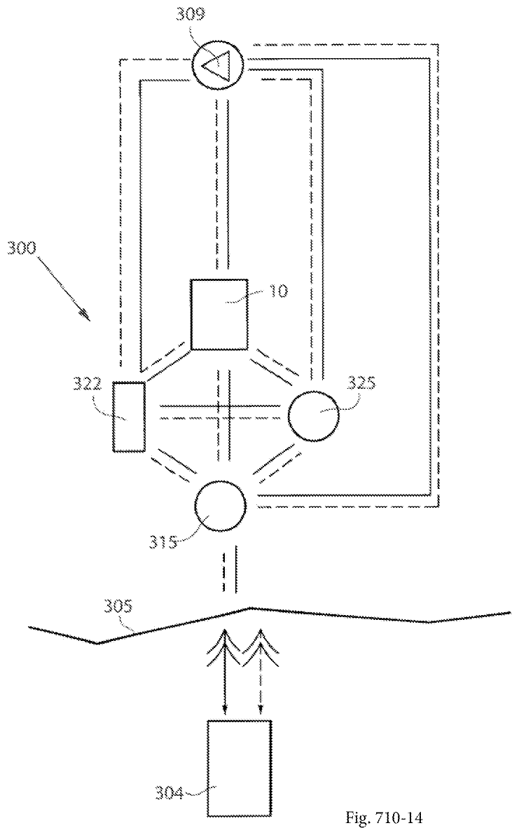

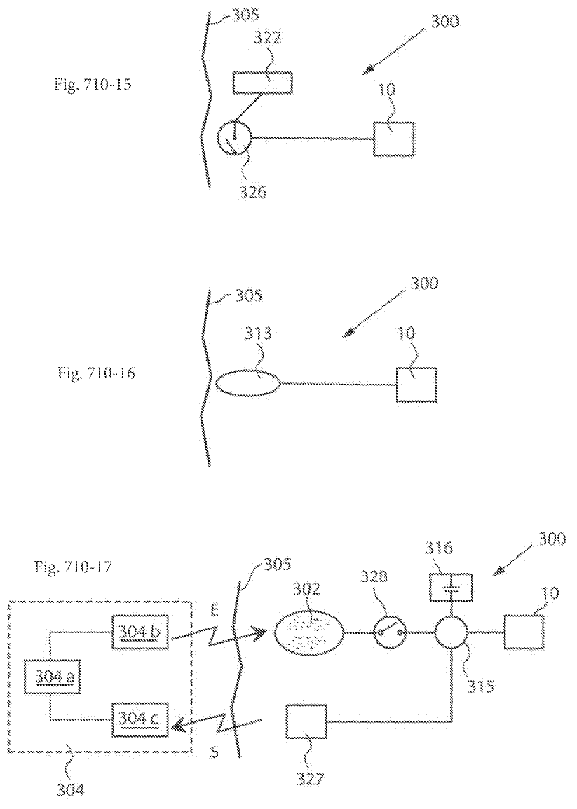

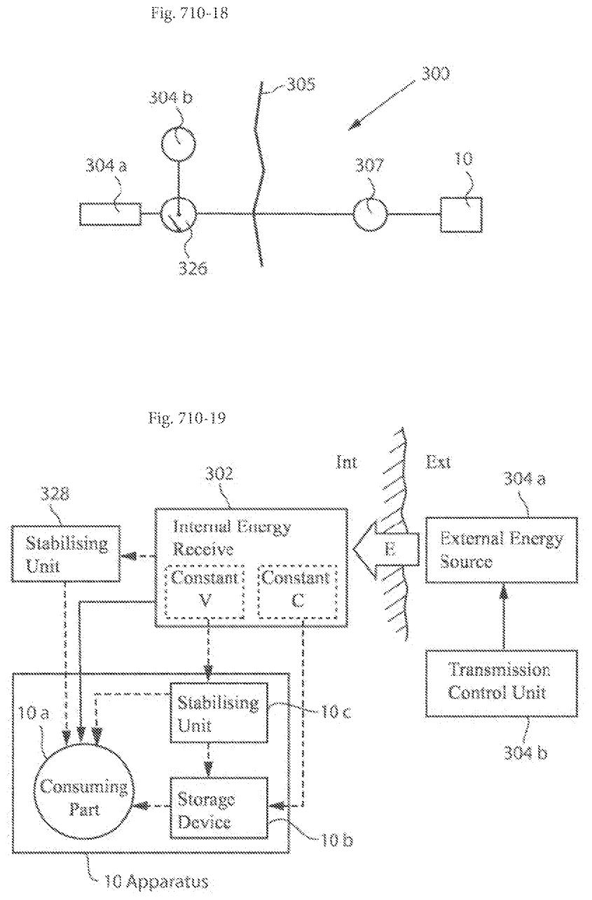

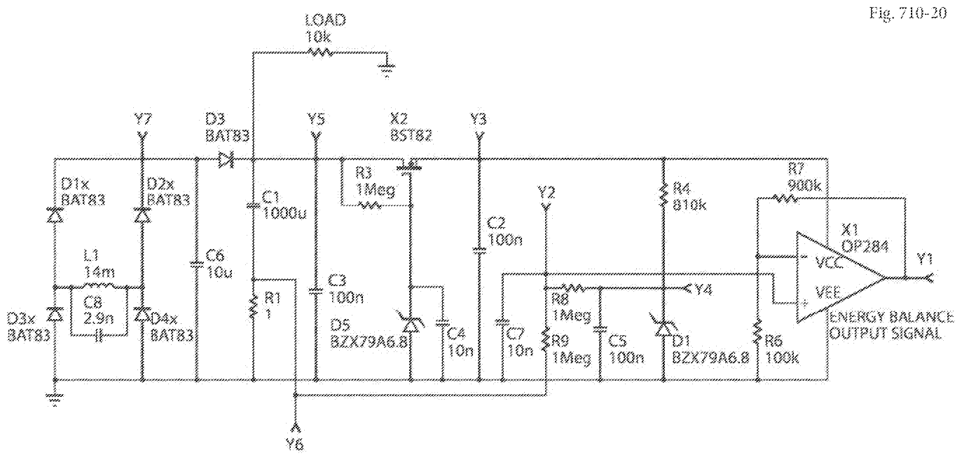

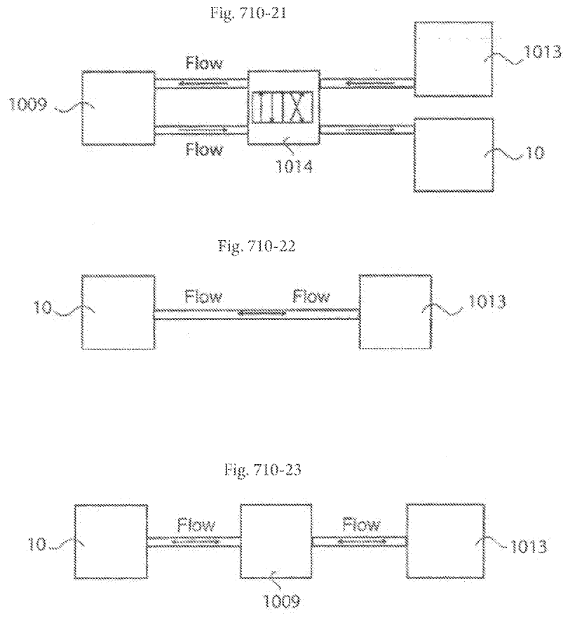

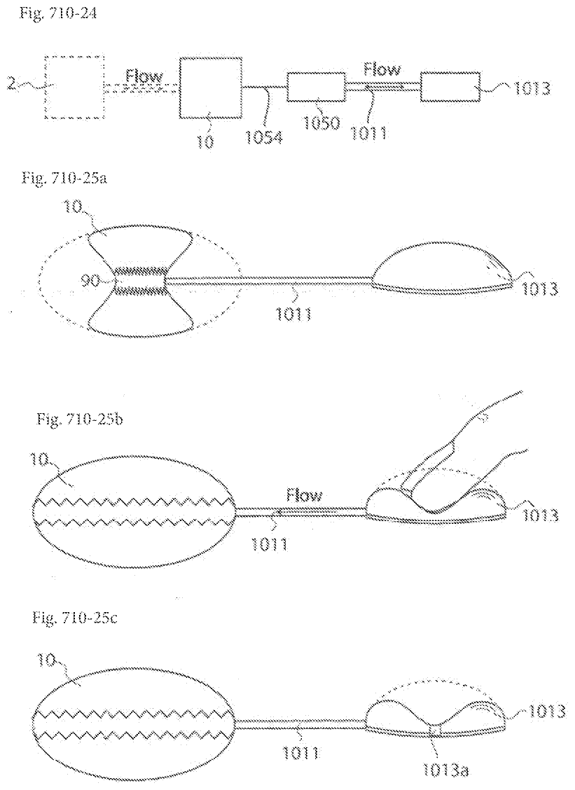

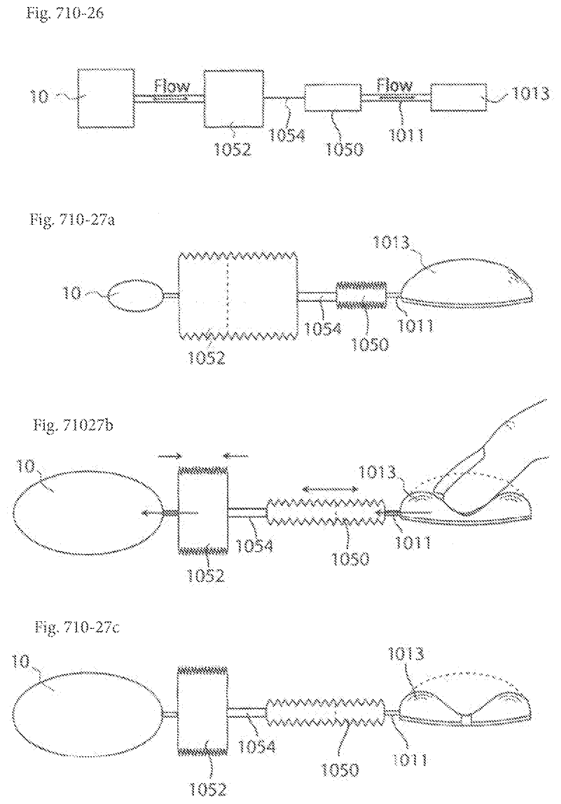

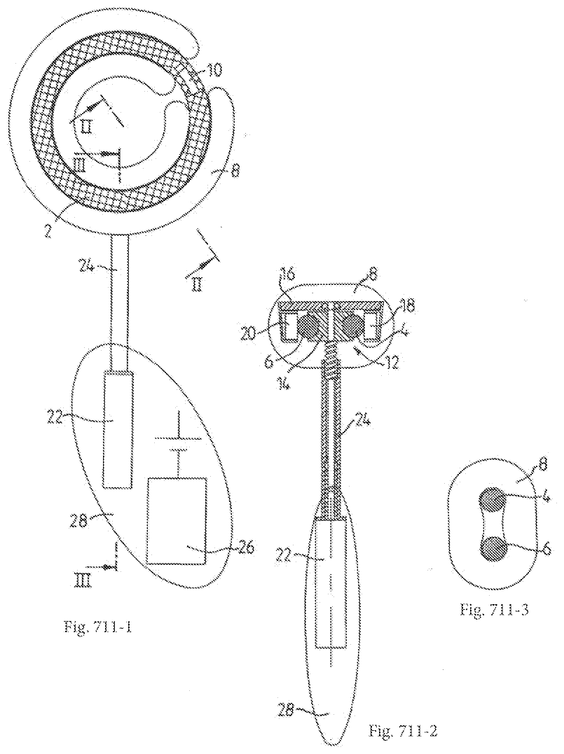

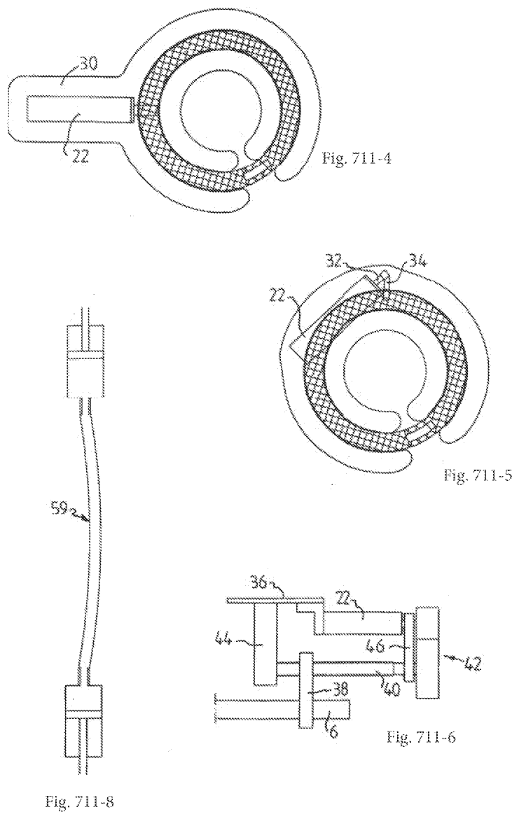

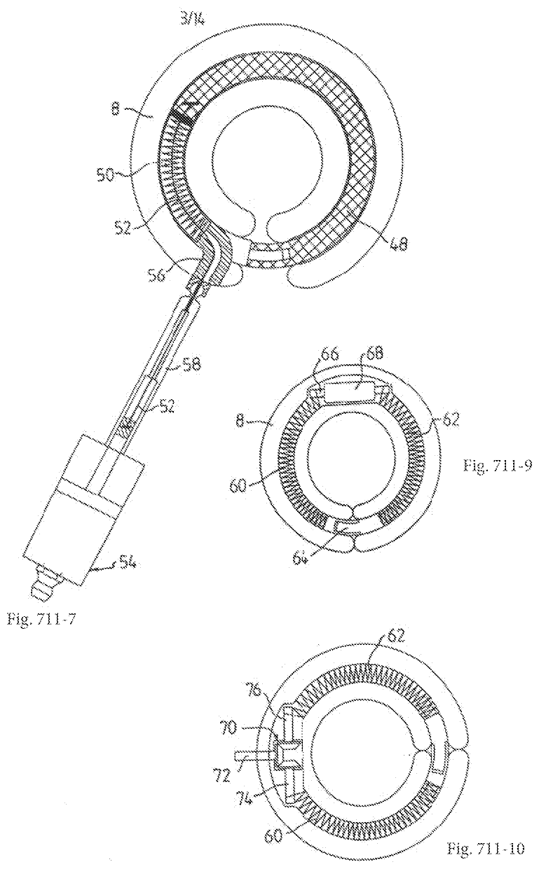

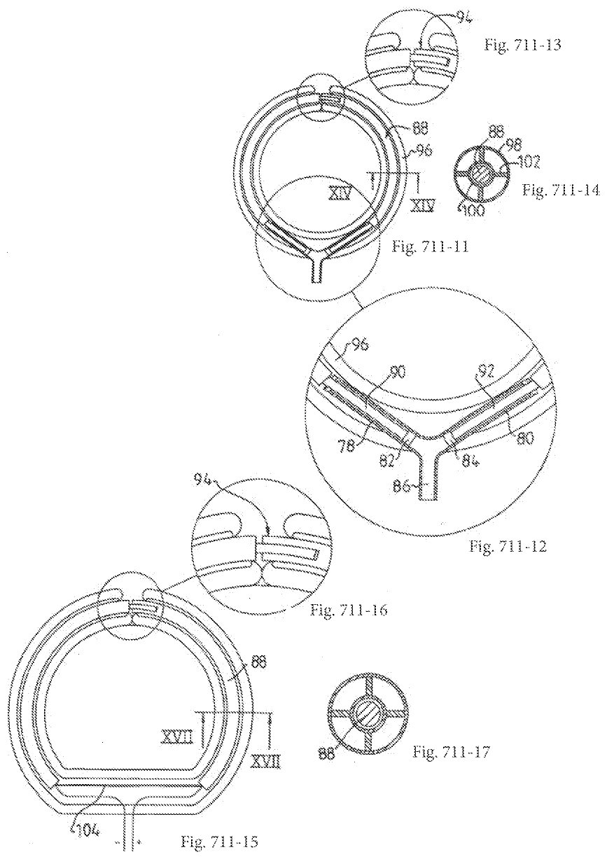

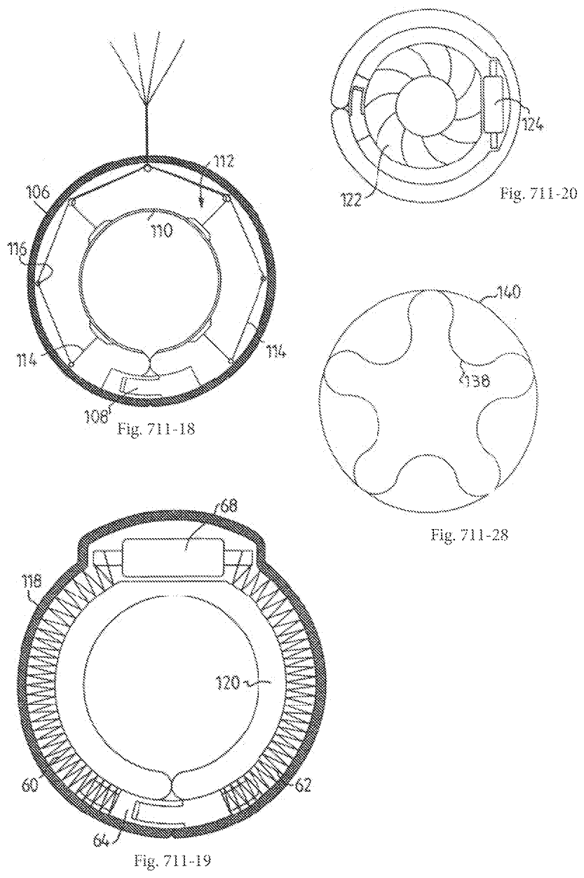

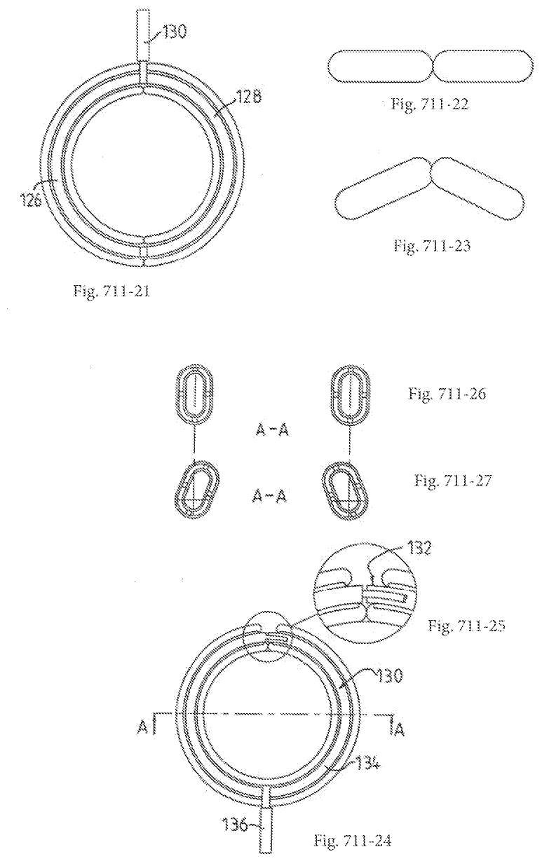

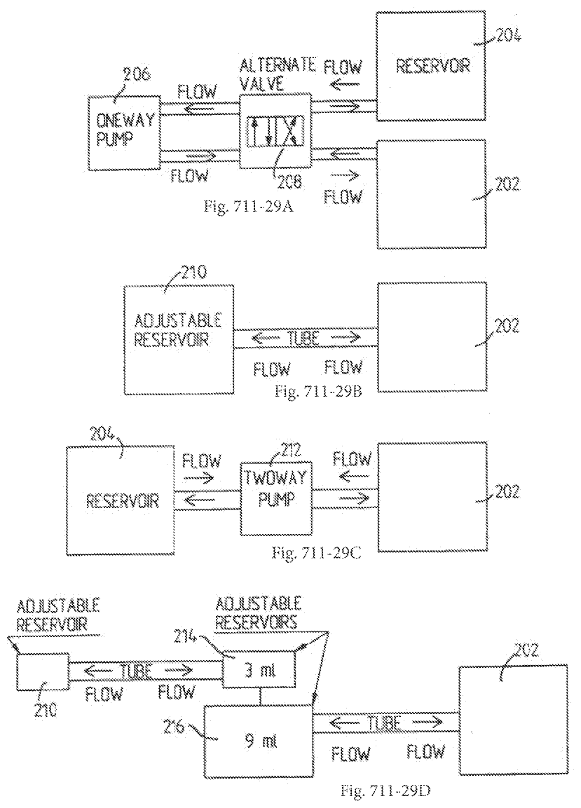

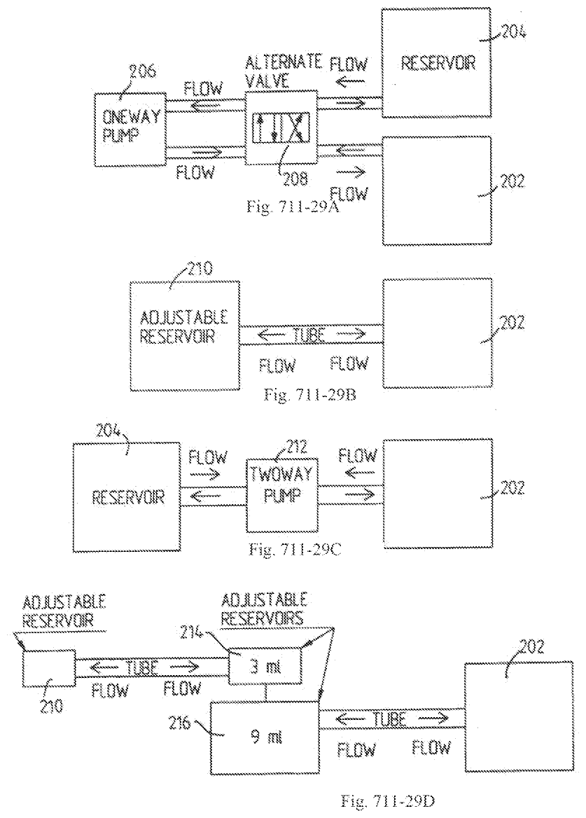

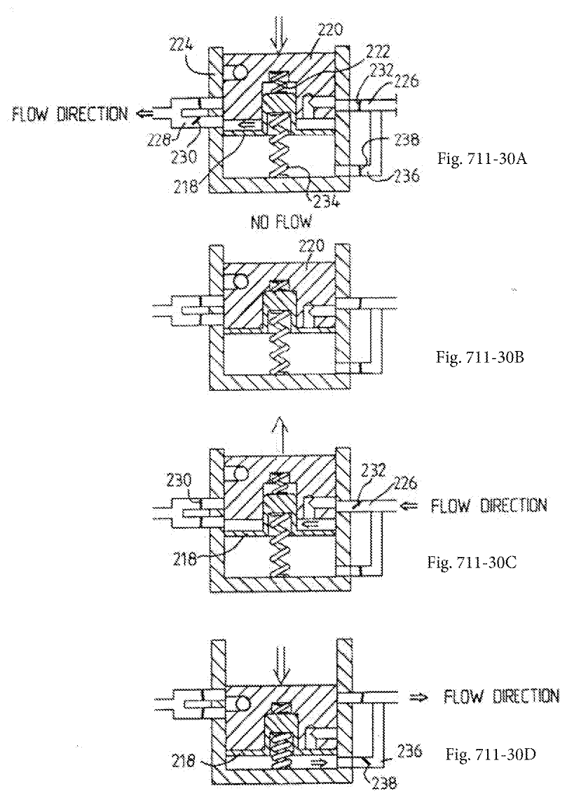

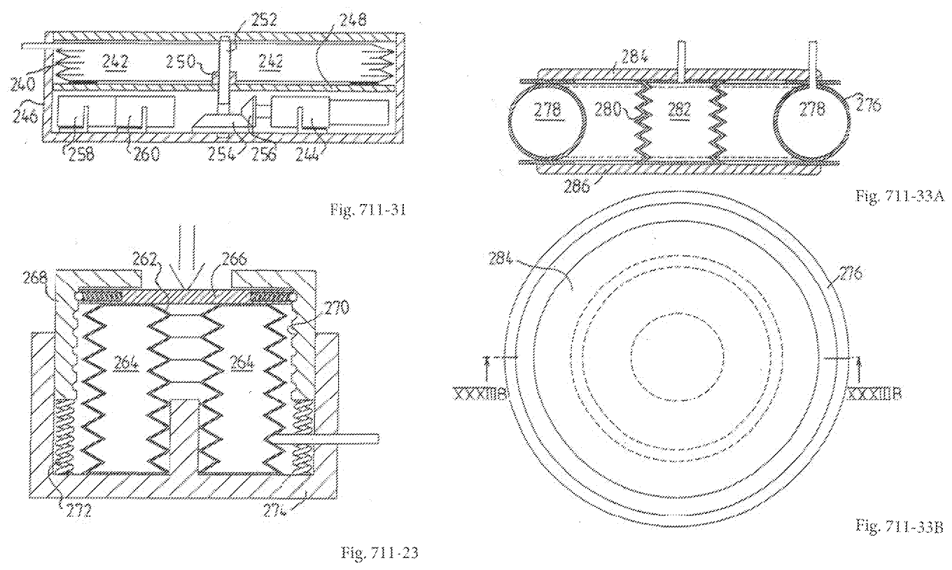

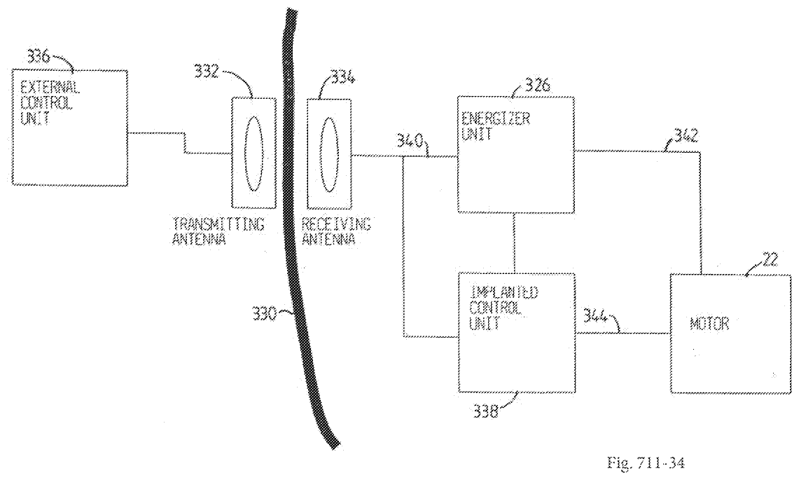

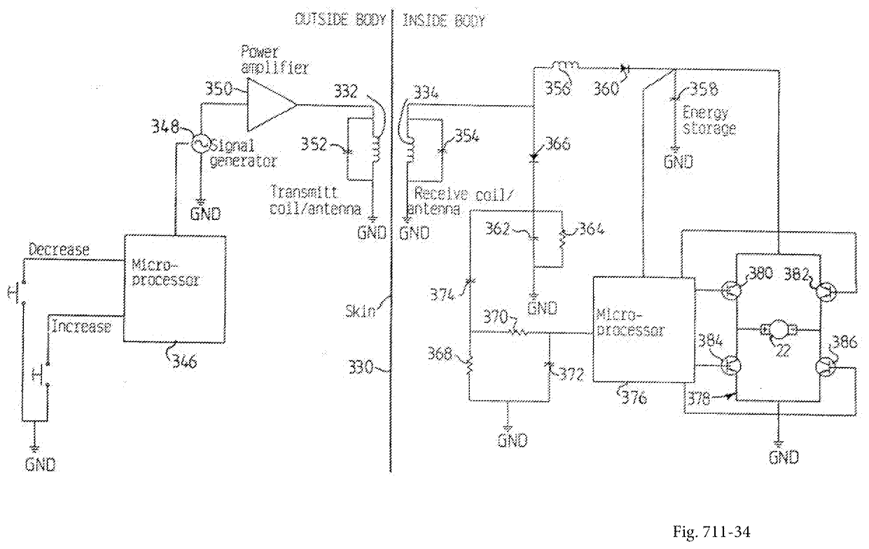

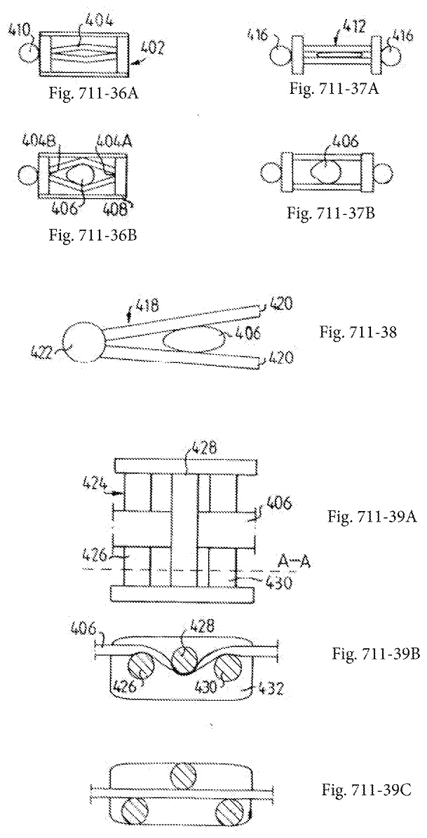

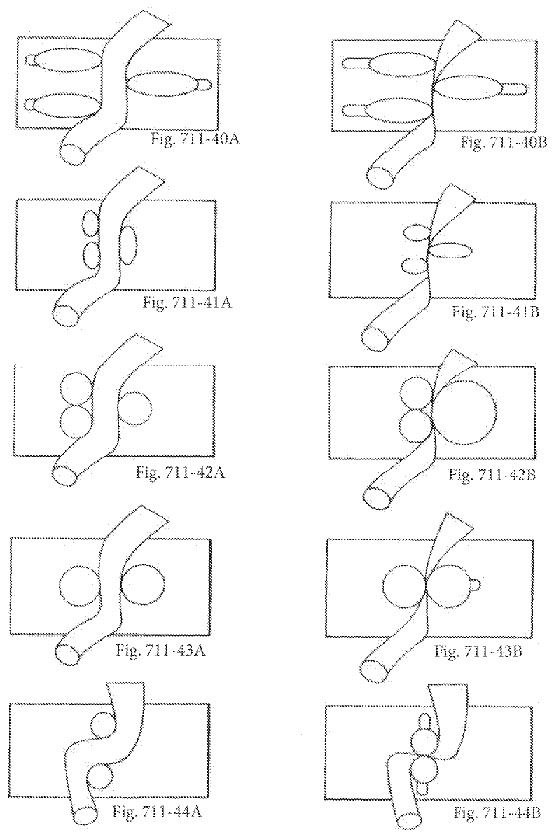

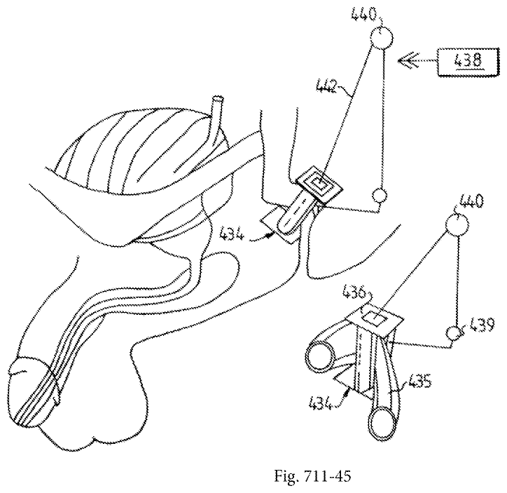

FIG. 137JJ shows a partially sectional perspective view from the left of an embodiment of a hydraulic pump for an implantable medical device.

FIG. 137KK shows a partially sectional perspective view from the left of an embodiment of a hydraulic pump for an implantable medical device.

FIG. 137MM shows a partially sectional perspective view from the left of an embodiment of a hydraulic pump for an implantable medical device.

FIG. 137NN shows a partially sectional perspective view from the left of an embodiment of a hydraulic pump for an implantable medical device.

FIG. 137PP shows a partially sectional perspective view from the left of an embodiment of a hydraulic pump for an implantable medical device.

FIG. 137QQ shows a partially sectional perspective view from the left of an embodiment of a hydraulic pump for an implantable medical device.

FIG. 137RR shows an elevated perspective view from the left of an embodiment of a hydraulic pump for an implantable medical device.

FIGS. 137SS-137UU, and 137XX-137ZZ show an embodiment and describes various functions of an implantable controller for controlling the implantable constriction device.

FIG. 137AAA shows a perspective elevated view from the right of an embodiment of an implantable energized medical device for powering an implantable medical device.

FIGS. 137BBB and 137CCC show lengthwise cross-sectional areas of the implantable medical device along the line A-A in FIG. 137AAA.

FIGS. 138-140 show cross-sectional plain side views of embodiments of an implantable energized medical device for powering an implantable medical device.

FIG. 141A shows a perspective elevated view from the right of an embodiment of an implantable energized medical device for powering an implantable medical device

FIGS. 141B and 141C show lengthwise cross-sectional areas of the implantable medical device along the line A-A in FIG. 141A.

FIG. 141D shows a cross-sectional plain side view of an embodiment of an implantable energized medical device.

FIGS. 141E-141G show cross-sectional plain side views of an embodiment of an implantable energized medical device.

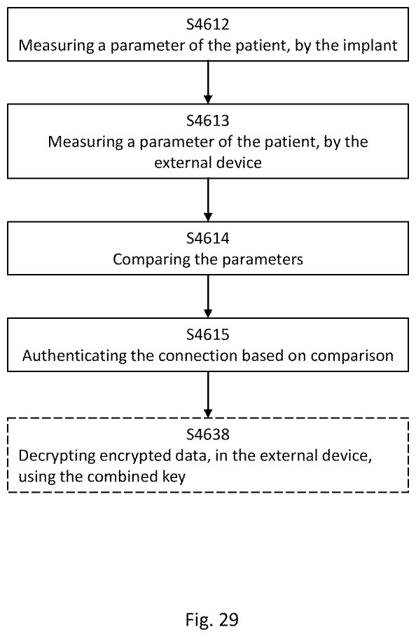

FIGS. 141H-141K shows cross-sectional plain side views of the embodiment in FIGS. 29a-29c when inserted into a tissue portion.

FIGS. 141M, 141N, 141P-141S show cross-sectional plain side views of embodiments of an implantable energized medical device.

FIG. 141T shows an embodiment of an implantable energized medical device for powering an implantable medical device or body engaging portion.

FIGS. 141U-141Z and 141AA-141JJ show schematic cross-sectional plain side views of systems comprising an implantable energized medical device.

FIGS. 141KK and 141MM show cross-sectional plain side views of embodiments of an implantable energized medical device.

FIGS. 141NN and 141PP show cross-sectional plain side views of embodiments of an implantable energized medical device.

FIG. 142 shows a system with an artificial intestine section implanted inside a patient's body and exiting a patient's stoma.

FIG. 143 shows the system of FIG. 142 with the energy being transmitted from an accumulator to the artificial intestine section.

FIG. 144 shows the system of FIG. 142 exiting the patient's anus.

FIG. 145 shows a system attached laterally to the patient's intestine.

FIG. 146 shows a system connected to a cross-sectional opening of the patient's intestine.

FIG. 147 shows an embodiment of the artificial intestine section with an artificial reservoir and an entry valve and exit.

FIGS. 148A and 148B show a first embodiment of the structure of FIG. 147.

FIGS. 149A and 149B show a system similar to the one of FIGS. 148A and 148B, wherein the entry and exit valves comprise bellows.

FIG. 150 shows an embodiment schematically, wherein the artificial intestine section by-passes a section of the patient's intestine.

FIGS. 151A to 151C show an embodiment, where the artificial intestine section comprises a reservoir with a flexible wall.

FIGS. 152A and 152B show a structure similar to the one of FIGS. 151A to 151C, wherein the pump and the reservoir are fixedly connected to one another.

FIGS. 153A to 153C show a plurality of cooperating valves implanted inside the patient's body and outside the patient's intestine.

FIGS. 154A to 154C show the stimulation devices of FIGS. 153A to 154C in combination with constriction devices.

FIGS. 155A and 155B show a system with the valve of FIG. 153A or 154A.

FIG. 156 shows the structure of the artificial intestine section for attachment to a lateral opening in the patient's intestine.

FIG. 157 shows a further structure of the artificial intestine section for attachment to a lateral opening in the patient's intestine.

FIG. 158 shows a ring-and-bulge connection.

FIGS. 159A and 159B show the ring-and-bulge connection of FIG. 158 in combination with a sleeve.

FIGS. 160A and 160B show a connection of the artificial intestine section to a cross-sectional opening of the patient's intestine without the bulge and the ring.

FIGS. 161A and 161B show an alternative connection of the artificial intestine section to a cross-sectional opening of the patient's intestine.

FIGS. 162A and 162B show another connection of the artificial intestine section to a cross-sectional opening of the patient's intestine.

FIG. 163A shows a surgically modified section of a human intestine forming an intestinal reservoir with a deactivated entry valve in front and an activated exit valve behind the intestinal reservoir.

FIG. 163B shows the intestinal reservoir of FIG. 163A with the entry valve activated and the exit valve deactivated.

FIG. 164A shows a plan view of an electrical type pump for emptying the intestinal reservoir of FIG. 163B with a plurality of rod-like electrical stimulation devices placed side by side adjacent the intestinal reservoir.

FIG. 164B shows a side view of the electrical type pump of FIG. 164A.

FIG. 165A shows a side view of a variant of the electrical type pump of FIG. 164B with the plurality of rod-like electrical stimulation devices placed side by side in folds formed by the wall of the intestinal reservoir.

FIG. 165B shows the variant of the electrical type pump of FIG. 165A in a different, cross-sectional side view.

FIG. 166A shows a plan view of a mechanical type pump for emptying the intestinal reservoir of FIG. 163B.

FIG. 166B shows a side view of the mechanical type pump of FIG. 166A.

FIG. 167A shows a plan view of a hydraulic type pump for emptying the intestinal reservoir of FIG. 163B.

FIG. 167B shows a side view of the hydraulic type pump of FIG. 167A.

FIG. 168 shows a strictly mechanical infusion device according to a first embodiment of the invention.

FIG. 169 shows the infusion device of FIG. 168 diagrammatically with some modifications.

FIG. 170 shows a cross sectional view of a strictly mechanical, completely implantable infusion device according to a second embodiment of the invention.

FIG. 171 shows a plan view of a part of the infusion device of FIG. 170, located adjacent a blood vessel.

FIG. 172 shows a cross sectional view of a penetration membrane made from a composite material.

FIG. 173 shows a motor-driven infusion device according to a third embodiment of the invention.

FIG. 174 shows a motor-driven pump unit suitable for use in connection with the embodiment shown in FIG. 173.

FIG. 175 shows a fully automatic unitary infusion device implanted subcutaneously adjacent a blood vessel.

FIG. 176 shows the muscles of the perineum.

FIG. 177 shows a cross-section through the penis.

FIG. 178 shows atop view of a first embodiment of the present disclosure including a single needle.

FIG. 179 shows atop view of a second embodiment of the present disclosure including a single needle and a motor accommodated in a common housing.

FIG. 180 shows a top view of a third embodiment of the present disclosure including two needles in a common housing.

FIG. 181 shows a plan view of a part of the drug delivery device of FIGS. 179 and 180.

FIG. 182 shows a cross-sectional view of a penetration membrane made from a composite material.

FIG. 183 shows a cross-sectional view through the outer wall with flaps in the penetration area.

FIG. 184 shows a cross-sectional view through the outer wall with an actively openable door in the penetration area.

FIG. 185 shows a cross-sectional view through the outer wall with an actively openable door according to another embodiment.

FIG. 186 shows a fourth embodiment including a plurality of needles within a common housing.

FIG. 187 shows a side view of a fifth embodiment of the present disclosure comprising a single needle which is laterally and vertically displaceable.

FIG. 188 shows a side view of a sixth embodiment of the present disclosure similar to the fifth embodiment, but with more steps for laterally displacing the needle.

FIG. 189 shows a seventh, spherical embodiment of the present disclosure for obtaining a three-dimensional array of penetration sites.

FIG. 190 shows a side view of an eighth embodiment of the present disclosure comprising two needles in a common housing which are laterally and vertically displaceable.

FIG. 191 shows a side view of a ninth embodiment of the present disclosure comprising two needles similar to the eighth embodiment, but with more steps for laterally displacing the needle.

FIG. 192 shows a tenth embodiment with a principle of advancing and retracting an infusion needle by means of a pull wire.

FIG. 193 shows an eleventh embodiment with a principle of laterally displacing an infusion needle by means of pull wires.

FIG. 194 shows a twelfth embodiment with a principle of advancing and retracting a needle and laterally displacing a needle by means of rotating shafts.

FIG. 195 shows the overall system of the present disclosure implanted in a patient's body according to a first variation.

FIG. 196 shows the overall system of the present disclosure implanted in the patient's body according to a second variation.

FIG. 197 shows the overall system of the present disclosure implanted in the patient's body according to a third variation.

FIG. 198 shows drug compartments as part of the reservoir of the system according to a first principle.

FIG. 199 shows drug compartments mounted on a tape wound on a reel in a replaceable cassette as part of the reservoir of the system according to a second principle.

FIG. 200 shows a part of the tape of FIG. 195 in greater detail.

FIG. 201 shows the principle of operation of the replaceable cassette of FIG. 199.

FIG. 202 shows drug compartments as part of the reservoir of the system according to a third principle.

FIG. 203 shows a cross-sectional view of the drug compartments of FIG. 202 including an insulation chamber and cooling device.

FIG. 204 shows the principle of the cooling device of FIG. 203 in combination with a heat exchanger.

FIG. 205 shows a specific embodiment for the cooling device of FIG. 203.

FIG. 206 shows a part of the system implanted in the patient's body comprising separate needles for the right and the left corpus cavernosum.

FIG. 207 diagrammatically shows the system of FIG. 206.

FIG. 208 diagrammatically shows the system of FIG. 205 with a long, flexibly bendable infusion needle.

FIG. 209 shows a part of the system of FIGS. 207 and 208, respectively, including a tube into which the needle can be advanced.

FIGS. 210A to 210C show a first and second embodiment for electromagnetically displacing the infusion needle in a plurality of lateral directions.

FIGS. 211a and 211b show a third embodiment for electromagnetically displacing the infusion needle in a plurality of lateral directions.

FIG. 212 shows the overall system of the present disclosure implanted in a patient's body according to a fourth variation.

FIG. 213 shows the overall system of the present disclosure implanted in the patient's body according to a fifth variation.

FIG. 214 shows the overall system of the present disclosure implanted in the patient's body according to a sixth variation.

FIG. 215 shows an embodiment including a single long and flexible infusion needle.

FIG. 216 shows an embodiment including a single infusion needle and a motor accommodated in a common housing.

FIG. 217 shows an embodiment including the tip ends of two long and flexible infusion needles in a common housing.

FIG. 218 shows an embodiment including the tip ends of a plurality of infusion needles within a common housing.

FIG. 219 shows a spherical embodiment for obtaining a three-dimensional array of penetration sites.

FIG. 220 shows an embodiment with a principle of advancing and retracting an infusion needle by means of a pull wire.

FIG. 221 shows an embodiment with a principle of laterally displacing an infusion needle by means of pull wires—

FIG. 222 shows an embodiment with a principle of advancing and retracting an infusion needle and laterally displacing an infusion needle by means of rotating shafts—

FIG. 223 shows the overall system implanted in a patient's body—

FIG. 224 shows the overall system implanted in the patient's body—

FIG. 225 shows the overall system implanted in the patient's body. FIG. 226 shows a patient's body with an implanted lubrication device for lubrication of a hip joint and/or a knee joint;

FIGS. 227A and 227B illustrate the hip joint and the knee joint of FIG. 226, respectively, having an infusion member of the implanted lubrication device inserted therein;

FIG. 227C shows a lateral view of a knee joint when a medical device has been provided.

FIG. 227D shows the medical device according to one embodiment, in section.

FIG. 227E illustrates the main components of an implanted lubrication device;

FIG. 227F shows a motor-driven implanted lubrication device establishing a circular flow path.

FIG. 228A shows an implanted lubrication device with an infusion needle and a drive mechanism.

FIG. 228B shows the lubrication device of FIG. 228A diagrammatically with some modifications.

FIG. 228C shows a cross sectional view of a compact embodiment of the implantable infusion device.

FIG. 228D shows a motor-driven pump unit suitable for use in connection with the embodiment shown in FIG. 227F.

FIG. 229 shows the medical device according to one embodiment comprising an artificial contacting surface.

FIG. 230 shows the medical device according to one embodiment comprising an artificial contacting surface, in section.

FIG. 231 shows a frontal view of a human patient displaying the hip joint.

FIG. 232 shows a lateral view of a human patient, in section, when a laparoscopic/arthroscopic procedure is being performed.

FIG. 233 shows the hip joint in section when a hole in the pelvic bone is being created.

FIG. 234 shows the hip joint in section when a small hole in the pelvic bone is being created.

FIG. 235 shows the hip joint in section when a medical device has been provided through a hole in the pelvic bone.

FIG. 236A shows the hip joint in section when a medical device is being provided through a hole in the pelvic bone.

FIG. 236B shows the hip joint in section when a medical device has been provided through a hole in the pelvic bone.

FIG. 237 shows the hip joint is section when a medical device connected to an implantable lubrication system is being provided.

FIGS. 238A-C show a surgical instrument for use in a method of providing a medical device according to any of the embodiments herein.

FIG. 239 shows the hip joint in section when a medical device has been implanted and connected to an implantable reservoir.

FIG. 240A shows the lateral view of a hip joint ion section when a hole is being created through the femoral bone.

FIG. 240B shows a hip joint in section when a medical device is being provided through a hole in the femoral bone.

FIG. 240C shows a hip joint in section when a medical device has been provided through a hole in the femoral bone.

FIG. 240D shows a reservoir adapted to be connected to a medical device, in further detail.

FIG. 241 shows the injection of a lubricating fluid into an implantable injection port.

FIG. 242 shows an implantable medical device in an opposite embodiment.

FIG. 243 shows a hip joint in section when an implantable medical device in an opposite embodiment has been placed.

FIG. 244 shows a hip joint in section when an implantable medical device in an opposite embodiment has been placed.

FIG. 245 shows a hip joint in section when an implantable medical device in an opposite embodiment has been placed and connected to a reservoir.

FIG. 246 shows a frontal view of a knee joint of a human patient.

FIG. 247 shows a frontal view of a knee joint of a human patient when a medical device has been provided.

FIG. 248 shows an implantable lubricating system.

FIG. 249A shows a lateral view of a knee joint when a medical device has been provided to the femoral bone.

FIG. 249B shows a lateral view of a knee joint when a medical device has been provided to the tibia bone.

FIG. 250 shows a medical device comprising an artificial knee joint surface.

FIG. 251 shows a medical device comprising an artificial knee joint surface in section.

FIG. 252 shows a medical device comprising multiple medical device parts.

FIG. 253 shows a medical device comprising multiple medical device parts, when assembled.

FIG. 254 shows the placing of a medical device comprising multiple medical device parts, when being fixated to the tibia bone.

FIG. 255 shows the implantable medical device according to an embodiment, when fixated to the tibia bone and connected to a reservoir and an injection port.

FIG. 256 shows a frontal view of a human patient when an implantable lubricating system has been provided.

FIG. 257 shows an implantable lubrication system in further detail.

FIG. 258 shows an implantable circling lubrication system in further detail.

FIG. 259 shows an implantable circling lubrication system comprising a filter, in further detail.

FIG. 260 shows an implantable lubrication system, when lubricating an artificial hip joint surface.

FIG. 261 shows an implantable lubrication system comprising a retractable needle, in a first state.

FIG. 262 shows an implantable lubrication system comprising a retractable needle, in a second state.

FIG. 263 (Prior art) shows an intramedullar device or internal auto distractor according to U.S. Pat. No. 5,156,605, inserted into the intramedullar cavity of a femur through an opening A in the epiphysis. The device is anchored by a pair of interlocking screws B at the top of the assembly, and by a pair of interlocking bolts C which will pass through the bottom of the assembly and secure it to the femur.

FIG. 264 (Prior art) shows an intramedullar pin or “medullar nail” according to EP 432 253 B1, also published as WO 91/00065, having securing holes E and D and a mechanical, pneumatic, hydraulic, electrical, or electromagnetic drive for rotating the rod for the longitudinal displacement of an inner portion thereof.

FIG. 265 (Prior art) schematically shows an external fixator (1) of the type frequently referred to as an Ilizarov apparatus, here consisting of two rings (2, 3) having pins (4) attached to and stabilizing the tibia or fibula in the lower leg (5) of a patient. The distance between the rings (2, 3) of the fixator (1) can be adjusted by manually turning threaded cylinders (6, 7, 8) on struts connecting the rings.

FIG. 266 shows one embodiment where two implanted devices are arranged to a bone.

FIG. 267 shows a detailed view of a device for bone adjustment according to an embodiment of the invention.

FIG. 268 schematically shows a device according to an embodiment of the inventions, implanted into the medullar cavity of a bone.

FIGS. 269A and 269B show detail views of devices according to embodiments of the invention.

FIG. 270A shows in detail 8a a schematic lateral view of the human spine, columna vertebralis (500) and illustrates in the partial views 270B and 270C how devices according to embodiments can be applied.

FIGS. 271A and 271B illustrate the straightening of a bone, or how the curvature of a bone can be adjusted, using devices according to embodiments of the invention.

FIGS. 271C and 271D show how the curvature of a bone, here a femur, can be adjusted using intramedullar devices according to embodiments of the invention.

FIGS. 271E and 271F illustrate how an intramedullar device can be used to adjust the torsion of a bone, here illustrated as the femur.

FIGS. 272A and 272B show schematically detail views of two devices according to embodiments of the invention, said devices including a mechanical multi step locking device.

FIG. 273 schematically illustrates a system for bone adjustment according to one embodiment of the invention, described in closer detail in the detailed description.

FIGS. 274A-274D schematically shows the insertion of a flexible device according to an embodiment of the invention.

FIGS. 275A-275E illustrate different non-limiting examples of the construction of the end-portions of the fastening means or anchoring devices according to embodiments of the invention.

FIG. 276 illustrates anchoring devices according to an embodiment of the invention.

FIG. 277 illustrates anchoring devices according to another embodiment of the invention.

FIG. 278 illustrates an embodiment of the device, comprising two telescopically arranged parts, housing a longitudinal threaded central shaft or axis and a motor or gear arrangement acting thereon, transforming rotational force into longitudinal force and extension or contraction of the device.

FIG. 279 shows a related embodiment where the device comprises three main parts, a central section, and two telescopically arranged end sections, each connected to a longitudinal threaded central shaft or axis through a motor or gear arrangement.

FIGS. 280A and 280B show embodiments where a device for bone adjustment according to the present invention is enclosed in a flexible, elastic, or expandable outer cover.

FIG. 281 illustrates an implanted device according to the invention, where the anchoring devices engage the bone from the inside of the medullar cavity.

FIG. 282 shows schematically an embodiment of a device comprising a motor, a gear box, and a speed controller.

FIG. 283 schematically shows an embodiment where the implanted adjustment device comprises at least two parts, wherein the parts are adapted to be positioned at an angle in relation to each other, and/or rotated in relation to each other.

FIGS. 284 and 285 are views of an implantable fluid movement device in accordance with a first embodiment,

FIG. 286 is a view of an implantable fluid movement device in accordance with a second embodiment, and

FIG. 287 is a flowchart illustrating different steps performed when implanting an implantable fluid movement device.

FIG. 288 is a sectional view of a cleaning device according to the invention.

FIG. 289 is a cross sectional view of the cleaning device of FIG. 288 taken along the line III-III before a cleaning operation.

FIG. 290 is a sectional view of the cleaning device of FIG. 288 taken along the line IV-IV.

FIG. 291 is a sectional view similar to that of FIG. 288 showing particles before a cleaning operation.

FIG. 292 is a sectional view similar to that of FIG. 288 during a first step of a cleaning operation.

FIG. 293 is a sectional view similar to that of FIG. 288 during a second step of a cleaning operation.

FIG. 294 is a sectional view similar to that of FIG. 288 during a third step of a cleaning operation.

FIG. 295 is a cross sectional view similar to that of FIG. 289 during a cleaning operation.

FIG. 296 is a sectional view of the cleaning device of FIG. 294 taken along the line X-X showing a cleaning ejection piston before ejection of particles.

FIG. 297 is a view similar to that of FIG. 295 but after ejection of particles.

FIG. 298 is a view of an alternative embodiment of a cleaning system.

FIG. 299 is a general view of an implanted drainage system in a patient.

FIG. 300 is a detailed view of a drainage system.

FIGS. 301A-301D are views of exemplary designs of tube ends for different treatment areas.

FIG. 302 is a view of a securing arrangement for securing a tube end in a bladder, such as the urine bladder.

FIG. 303A is a circuit diagram showing an energy transfer amplifier, where the energy is transferred by ultrasonic waves.

FIG. 303B-303C is a circuit diagram showing further another embodiment of an amplifier.

FIG. 303D-303E are graphs showing different waveforms of signals in the amplifier of the ultrasonic embodiment.

FIG. 304 is general view of an implanted drainage apparatus with a filter in a patient.

FIG. 305 is a detailed view of a powered filter.

FIGS. 306 and 307 are views of a filter cassette.

FIGS. 308 and 309 are views of a filter cassette.

FIG. 310 shows the placement in the body of a hydraulic treatment embodiment.

FIG. 311-312 describes a hydraulic treatment embodiment for emptying the urine bladder.

FIG. 313 shows an implantable device for improving the pump function of the heart in a lateral view.

FIG. 314 shows an implantable device for improving the pump function of the heart in a frontal view.

FIG. 315 shows an implantable device for improving the pump function of the heart in a lateral view.

FIG. 316 shows an implantable device for improving the pump function of the heart in a lateral view.

FIG. 317 shows an implantable device for improving the pump function of the heart in a frontal view.

FIG. 318 shows an implantable device for improving the pump function of the heart in a lateral view.

FIG. 319 shows an operating device in detail.

FIG. 320 shows an operating device in detail.

FIG. 321 shows an implantable device for improving the pump function of the heart in a lateral view.

FIG. 322 shows an implantable device for improving the pump function of the heart in a lateral view.

FIG. 323 shows an implantable device for improving the pump function of the heart in a frontal view.

FIG. 324 shows an implantable device for improving the pump function of the heart in a frontal view.

FIG. 325 shows an implantable device for improving the pump function of the heart in a lateral view.

FIG. 326 shows, schematically, a system for transferring force.

FIG. 327 shows, schematically, a system for transferring force.

FIG. 328 shows, schematically, a system for transferring force.

FIG. 329 shows, schematically, how force is exerted on a heart.

FIG. 330 shows, schematically, how force is exerted on a heart.

FIG. 331 shows, schematically, how force is exerted on a heart.

FIG. 332 shows, schematically, how force is exerted on a heart.

FIG. 333 shows an implantable device for improving the pump function of the heart in a frontal view.

FIG. 334 shows an implantable device for improving the pump function of the heart in a lateral view.

FIG. 335 shows an implantable device for improving the pump function of the heart in a lateral view.

FIG. 336 shows an implantable device for improving the pump function of the heart in a frontal view.

FIG. 337 shows an implantable device for improving the pump function of the heart in a lateral view.

FIG. 338 shows, schematically, a system for transferring force.

FIG. 339 shows, schematically, a system for transferring force.

FIG. 340 shows, schematically, an operating device and a fixating member.

FIG. 341 shows, schematically, a system for transferring force.

FIG. 342 shows a frontal view of a human patient with an LVAD.

FIG. 343 shows an implanted artificial heart device in a lateral view.

FIG. 344 shows, schematically, a system for transferring force.

FIG. 345 shows, schematically, a system for transferring force.

FIG. 346 shows a frontal view of a human patient with an implanted system for transferring force.

FIG. 347 shows, schematically, a system for transferring force.

FIG. 348 shows, schematically, a system for transferring force.

FIG. 349 shows, schematically, a system for transferring force.

FIG. 350 shows a heart contacting organ in a first position.

FIG. 351 shows a heart contacting organ in a second position.

FIG. 352 shows a heart contacting organ in detail.

FIG. 353 shows a heart contacting organ in detail.

FIG. 354 shows a device for adjusting a heart contacting organ in a first position.

FIG. 355 shows a device for adjusting a heart contacting organ in a second position.

FIG. 356 shows a heart of a human patient in a frontal view.

FIG. 357 shows a system for adjusting the position of a pump device in a first position.

FIG. 358 shows a system for adjusting the position of a pump device in a second position.

FIG. 359 shows a fixation system.

FIG. 360 shows a fixation system.

FIG. 361 shows a fixation system.

FIG. 362 shows a fixation system.

FIG. 363 shows a fixation system.

FIG. 364 shows a fixation system.

FIG. 365 shows a frontal view of the sternum of a human patient, with a fixating system applied.

FIG. 366 shows a frontal view of the rib cage of a human patient, with a fixating system applied.

FIG. 367 shows a frontal view of the rib cage of a human patient, with a fixating system applied.

FIG. 368 shows a frontal view of the rib cage of a human patient, with a fixating system applied.

FIG. 369 shows a frontal view of the rib cage of a human patient, with a fixating system applied.

FIG. 370 shows a lateral view of the vertebral column of a human patient, with a fixating system applied.

FIG. 371 shows a lateral view of the vertebral column of a human patient, with a fixating system applied.

FIG. 372 shows a frontal view of a part of the vertebral column of a human patient, with a fixating system applied.

FIG. 373 shows an implantable device for improving the pump function of the heart in a lateral view.

FIG. 374 shows a sealed chamber comprising an operating device.

FIG. 375 shows a sealed chamber for hydraulic use.

FIG. 376 shows a lateral view of a patient when a heart help device is fixated to the sternum of the patient, on the inside thereof.

FIG. 377 shows a lateral view of a patient when a heart help device is fixated to a vertebra of the patient.

FIG. 378 shows a lateral view of a patient when a heart help device is fixated to a rib of the patient.

FIG. 379 shows a lateral view of a patient when a heart help device is fixated to the sternum of the patient on the inside thereof, in a diaphragm penetrating way.

FIG. 380 shows a lateral view of a patient when a heart help device is fixated to the sternum of the patient, on the outside thereof.

FIG. 381 shows a lateral view of a patient when a diaphragm contacting part is placed.

FIG. 382 shows a lateral view of a patient when an opening is created in the thorax of the patient.

FIG. 383 shows a close-up of a diaphragm contacting part maintaining an opening in the thoracic diaphragm.

FIG. 384 shows an embodiment of a heart help device where force is transferred through the thoracic diaphragm.

FIG. 385 shows a second embodiment of a heart help device where force is transferred through the thoracic diaphragm.

FIG. 386 shows an alternative embodiment of the respiratory movement compensator.

FIG. 387 shows an alternative embodiment of the respiratory movement compensator in a second state.

FIG. 388 shows a second embodiment of a heart help device where mechanical and hydraulic force is transferred through the thoracic diaphragm.

FIG. 389 shows a first embodiment of a multi-chamber injection port for calibrating elements pressing on the heart.

FIG. 390 shows a second embodiment of a multi-chamber injection port.

FIG. 391 shows a hydraulic/pneumatic two chamber system.

FIG. 392 shows a hydraulic/pneumatic system comprising a selection valve.

FIG. 393 shows a hydraulic/pneumatic closed force transferring chamber system comprising a selection valve.

FIG. 394 shows an embodiment of a heart help device in which hydraulic force is transferred through the thoracic diaphragm.

FIG. 395A shows an embodiment of a diaphragm contacting part in which the diaphragm contacting part is adapted to be opened, in an open state.

FIG. 395B shows an embodiment of a diaphragm contacting part in which the diaphragm contacting part is adapted to be opened, in a closed state.

FIG. 395C shows an embodiment of a diaphragm contacting part, which is not possible to open.

FIG. 395D shows an embodiment of a diaphragm contacting part, in section.

FIG. 396 shows a diaphragm contacting part, with a force transferring member for transferring of mechanical force placed inside.

FIG. 397 shows a diaphragm contacting part, with two force transferring member for transferring of mechanical force placed inside.

FIG. 398 shows a diaphragm contacting part, with a force transferring member creating a sealing with the diaphragm contacting part placed inside.

FIG. 399 shows a diaphragm contacting part, with a force transferring member for transferring of hydraulic force placed inside.

FIG. 400 shows a diaphragm contacting part, with one force transferring member for transferring of hydraulic, and one force transferring member for transferring hydraulic force placed inside.

FIG. 401 shows a force transferring part for transferring force through the thoracic diaphragm.

FIG. 402A shows a displaceable heart help device in a first perspective view.

FIG. 402B shows a displaceable heart help device in a second perspective view.

FIG. 403 shows a magnetic operating device in section.

FIG. 404 shows a heart help device comprising a magnetic operating device in a perspective view.

FIG. 405 shows a displaceable heart help device in a first perspective view.

FIG. 406A shows a heart help device adapted to be inserted through an opening in the thoracic diaphragm, in its folded state.

FIG. 406B shows a heart help device adapted to be inserted through an opening in the thoracic diaphragm, in its unfolded state.

FIG. 407 shows a flow-chart of an operation method for fixation a heart help device.

FIGS. 408 to 419 are schematic block diagrams illustrating twelve embodiments, respectively, of the male impotence prosthesis apparatus of the invention, in which wireless energy is transmitted from outside a patient's body to energy consuming components of the apparatus implanted in the patient.

FIG. 420 is a schematic block diagram illustrating conceivable combinations of implanted components for achieving various communication options;

FIG. 421 illustrates an electrical junction element for use in the apparatus of the present invention; and

FIG. 422 illustrates the apparatus in accordance with the invention implanted in a patient;

FIG. 423 is a block diagram illustrating remote control components of an embodiment of the invention, in which wireless energy is transmitted by the use of electromagnetic signals; and

FIG. 424 is a schematic view of exemplary circuitry used for the components of the block diagram of FIG. 423.

FIG. 425 is general view of a human body having a device for treating aneurysm implanted,

FIG. 426 is a view illustrating a device for treating aneurysm with associated equipment,

FIG. 427 is a view illustrating a mechanical device for treating aneurysm,

FIG. 428 is a view illustrating a mechanical device for treating aneurysm,

FIG. 429 is a view illustrating a hydraulic device for treating aneurysm,

FIG. 430 is a view illustrating a hydraulic device for treating aneurysm,

FIG. 431 is a view illustrating a hydraulic device for treating aneurysm,

FIG. 432 is a view illustrating a stimulation device for treating a vascular aneurysm of a human or mammal patient,

FIG. 433 is a view illustrating a sensor used when treating or monitoring a vascular aneurysm of a human or mammal patient,

FIG. 434 is a view illustrating a device for treating aneurysm. FIG. 435 is a view illustrating a device for treating aneurysm.

FIG. 436 is an overview of the body of a patient having an implanted heart pump.

FIG. 437 is a sectional view of a clot removal device according to the invention.

FIG. 438 is a cross sectional view of the clot removal device of FIG. 437 taken along the line III-III before a cleaning operation.

FIG. 439 is a sectional view of the clot removal device of FIG. 437 taken along the line IV-IV.

FIG. 440 is a sectional view similar to that of FIG. 437 showing blood clots before a clot removal operation.

FIG. 441 is a sectional view similar to that of FIG. 437 during a first step of a clot removal operation.

FIG. 442 is a sectional view similar to that of FIG. 437 during a second step of a clot removal operation.

FIG. 443 is a sectional view similar to that of FIG. 437 during a third step of a clot removal operation.

FIG. 444 is a cross sectional view similar to that of FIG. 438 but during a cleaning operation.

FIG. 445 is a sectional view of the clot removal device of FIG. 443 taken along the line X-X showing a clot ejection piston before ejection of clots.

FIG. 446 is a view similar to that of FIG. 444 but after ejection of clots.

FIG. 447 is an overall view of a clot removal system according to the invention.

FIGS. 448 and 449 are views of a filter cassette.

FIGS. 450A and 450B are views of a filter cassette.

FIG. 451 shows a schematic cross-sectional view of an embodiment of the apparatus of invention when implanted in a patient.

FIGS. 452 and 453 schematically show an embodiment of the apparatus with a first variant of the powered member.

FIGS. 454 and 455 schematically show respectively different embodiments of the pressurizer of the powered member.

FIG. 456 is a schematic picture of patient having the inventive apparatus implanted.

FIG. 457A is a schematic picture of an embodiment of the apparatus as implanted.

FIG. 457B is a section of the apparatus FIG. 457a illustrating the detachable coupling between parts of the apparatus.

FIG. 458 is an illustration of the apparatus of FIG. 457A in its operating mode of discharging urine from the urinary bladder through the urethra.

FIG. 459 is an illustration of the apparatus FIG. 457A when the urine bladder is refilled with urine, also showing a special embodiment where the bladder operating reservoir is hydraulically connected to the ureter restriction devices.

FIG. 460 illustrates another embodiment of the apparatus.

FIGS. 461A to 461C show a breast implant system according to a first embodiment of the present invention,

FIGS. 462 to 464 show a breast implant system according to a second embodiment of the present invention,

FIGS. 465A to 465B show a breast implant system according to a third embodiment of the present invention,

FIGS. 466 to 467 show a breast implant system according to a fourth embodiment of the present invention,

FIGS. 468A to 468B show a breast implant system according to a fifth embodiment of the present invention,

FIGS. 469A to 469B show a breast implant system according to a sixth embodiment of the present invention,

FIGS. 470A to 470B show a breast implant system according to a seventh embodiment of the present invention,

FIGS. 471A to 471B show a breast implant system according to an eighth embodiment of the present invention,

FIGS. 472A to 472C show a breast implant system according to a ninth embodiment of the present invention,

FIG. 473 shows a breast implant system according to a tenth embodiment of the present invention,

FIG. 474 shows a breast implant system according to an eleventh embodiment of the present invention,

FIGS. 475A to 475B show a breast implant system according to a twelfth embodiment of the present invention, and

FIGS. 476A to 476B show a breast implant system according to a thirteenth embodiment of the present invention.

FIG. 477 is an overall view of a patient showing the outlines of the stomach,

FIG. 478 is a view of a first embodiment of an apparatus for treating obesity implanted in a human patient,

FIG. 479 is a sectional view taken along line IIb-IIb of FIG. 478,

FIGS. 480A-K, 480M, 480N, 480P show different shapes and features of a volume filling device comprised in an apparatus according to the invention,

FIGS. 481A-481D show a deflated inflatable volume filling device comprised in an apparatus according to the invention and an instrument for placing the volume filling device,

FIGS. 482A-482I illustrate different steps of invaginating the inflatable device of FIG. 481A on the outside of a stomach wall of a patient,

FIGS. 483A, 483b, 484 and 485 show alternative embodiments wherein the volume filling device is adapted to be non-invasively adjustable postoperatively,

FIGS. 486 and 487 show embodiments wherein the volume filling device is adapted to be invaginated in the fundus region of the patient's stomach,

FIG. 488 shows an embodiment wherein the volume filling device is also adapted to treat reflux,

FIG. 489 show an embodiment wherein the volume filling device adapted also for treating reflux is combined with stretching devices for stretching part of the stomach fundus wall,

FIGS. 490-493 show alternative embodiments wherein a combination of a volume filling device and a stretching device is used,

FIGS. 494 and 495 show an embodiment wherein the volume filling device is provided on the inside of the stomach wall,

FIGS. 496A-496H illustrate different steps of invaginating the inflatable device of FIG. 481A on the inside of a stomach wall of a patient,

FIGS. 497A-497J illustrate different steps of invaginating the inflatable device of FIG. 481A on the inside of a stomach wall of a patient,

FIGS. 498A-498F illustrate different steps of invaginating the inflatable device of FIG. 481A on the inside of a stomach wall of a patient,

FIGS. 499A and 499B show an instrument used in a method of engaging a volume filling device.

FIG. 500 illustrate the invagination of a plurality volume filling devices, and

FIGS. 501A and 501B illustrate an abdominal method.

FIG. 502A illustrates a system according to the invention applied on the oviducts of a female patient, wherein restriction devices are in a non-restricting operating state.

FIG. 502B is a view similar to that of FIG. 502A, but wherein restriction devices are in a restricting operating state.

FIG. 503A illustrates a system according to the invention with remote control applied on the oviducts of a female patient, wherein restriction devices are in a non-restricting operating state.

FIG. 503B is a view similar to that of FIG. 503A, but wherein restriction devices are in a restricting operating state.

FIG. 504A is a schematic view of a hydraulic operation means with a subcutaneously placed reservoir suited for operating the restriction device of the embodiments of FIGS. 502A, 502B.

FIG. 504B shows the embodiment of FIG. 504A with the constriction device constricting a tissue wall of a patient's oviduct.

FIG. 505A is a schematic view of mechanical operation means suited for operating the constriction device of the embodiments of FIGS. 503-512.

FIG. 505B shows the embodiment of FIG. 505A with the constriction device constricting a tissue wall of a patient's organ.

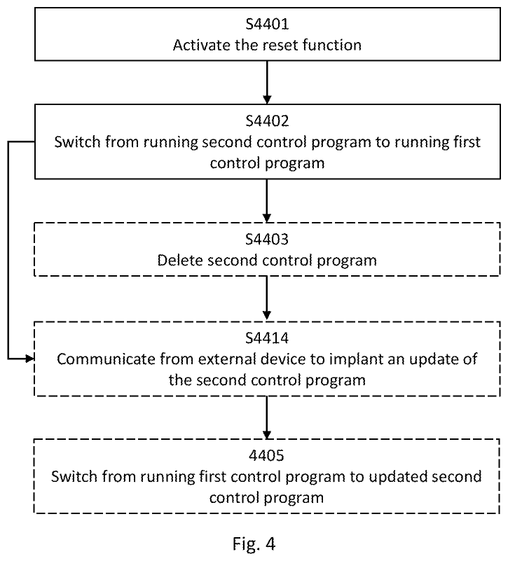

FIG. 505C shows a modification of the embodiment of FIG. 04B.

FIGS. 506A, 506B and 506C are cross-sections of the embodiment of FIG. 503 showing different states of operations with the system applied on a tissue wall of a patient's organ.

FIGS. 507A, 507B and 507C are cross-sections of a modification of the embodiment of FIG. 503 showing different states of operations with the system applied on a tissue wall of a patient's organ.

FIGS. 508A, 508B, 508C, 508D and 508E schematically illustrate different states of operation of a general embodiment of a system according to the present invention.

FIGS. 509A, 509B and 509C illustrate different states of operation of a modification of the general embodiment.

FIGS. 509D, 509E and 509F illustrate an alternative mode of operation of the modification of the general embodiment.

FIG. 510A is a pulse/time diagram showing electric stimulation pulses generated by the system for stimulating a tissue wall of a patient's organ.

FIG. 510B is pulse/time diagram showing a modification of the electric stimulation shown in FIG. 510a, in which pulses of mixed frequencies and/or amplitudes are employed.

FIGS. 510C and 510D show two pulse/time diagrams, respectively, representing electric stimulation of two different areas of the tissue wall with pulses forming pulse trains.

FIG. 511A is a longitudinal cross-section of an embodiment of the system including a thermal stimulation device, wherein the system is constricting a tissue wall of a patient's oviduct.

FIG. 511B is the same embodiment of FIG. 511A with the thermal stimulation device activated.

FIG. 512A is a schematic view of a hydraulically operable inflatable constriction device for use in accordance with the invention.

FIG. 512B is the same embodiment shown in FIG. 512A with the constriction device inflated.

FIGS. 513A, 513B, 513C and 513D are block diagrams illustrating four different principles for hydraulic operation of the constriction device shown in FIG. 512A.

FIG. 514 is a cross-sectional view of a reservoir having a variable volume controlled by a remote control motor.

FIGS. 515A and 515B are perspective views of a reverse servo in accordance with a particular embodiment of the hydraulic operation principle shown in FIG. 513C.

FIG. 516 is a schematic view of another hydraulically operable constriction device for use in accordance with the invention.

FIG. 517A illustrates a constriction device in a constricted state.

FIG. 517B illustrates the constriction device of FIG. 516 in a released state.

FIG. 518 is a schematic block diagram illustrating a general embodiment of the system of the invention, in which energy is transferred to energy consuming components of the system implanted in the patient.

FIGS. 519 to 530 are schematic block diagrams illustrating twelve embodiments, respectively, wherein wireless energy is transmitted from outside a patient's body to energy consuming components of the system implanted in the patient.

FIG. 531 is a block diagram illustrating control components of an embodiment of the invention.

FIG. 532 is a schematic view of exemplary circuitry of an embodiment of the invention, in which wireless energy is transformed into a current.

FIGS. 533A, 533B, 533C, 533D and 533E schematically illustrate different states of operation of a general embodiment of an apparatus according to the present invention.

FIGS. 533F, 533G and 533H illustrate different states of operation of a modification of the general embodiment.

FIGS. 533I, 533K and 533L illustrate an alternative mode of operation of the modification of the general embodiment.

FIG. 534 is a longitudinal cross-section of a preferred embodiment of the apparatus according to the invention including a constriction device and an electric stimulation device.

FIG. 535 is a cross-section along line III-III in FIG. 534.

FIG. 536 is the same cross-section shown in FIG. 535, but with the apparatus in a different state of operation.

FIGS. 537A, 537B and 537C are cross-sections of the embodiment of FIG. 534 showing different states of operations with the apparatus applied on a uterine tube wall of a patient's uterine tube.

FIGS. 538A, 538B and 538C are cross-sections of a modification of the embodiment of FIG. showing different states of operations with the apparatus applied on a uterine tube wall of a patient's uterine tube.

FIGS. 539A and 539B show different steps of an electric stimulation mode performed by the apparatus of FIG. 534, while the apparatus is constricting a uterine tube wall of a patient's uterine tube.

FIG. 540A is a pulse/time diagram showing electric stimulation pulses generated by the apparatus for stimulating a uterine tube wall of a patient's uterine tube.

FIG. 540B is pulse/time diagram showing a modification of the electric stimulation shown in FIG. 540A, in which pulses of mixed frequencies and/or amplitudes are employed.

FIGS. 541A and 541B show two pulse/time diagrams, respectively, representing electric stimulation of two different areas of the uterine tube wall with pulses forming pulse trains.

FIGS. 542A and 542B show the pulse/time diagrams of FIGS. 541A and 541B with modified pulse trains.

FIG. 543A is a longitudinal cross-section of an embodiment of the apparatus including a thermal stimulation device, wherein the apparatus is constricting a uterine tube wall of a patient's uterine tube.

FIG. 543B is the same embodiment of FIG. 543A with the thermal stimulation device activated.

FIG. 544A is a schematic view of hydraulic operation means suited for operating the constriction device of the embodiments of FIGS. 534-543.

FIG. 544B shows the embodiment of FIG. 544a with the constriction device constricting a uterine tube wall of a patient's uterine tube.

FIG. 545A is a schematic view of mechanical operation means suited for operating the constriction device of the embodiments of FIGS. 534-543.

FIG. 545B shows the embodiment of FIG. 545A with the constriction device constricting a uterine tube wall of a patient's uterine tube.

FIG. 545C shows a modification of the embodiment of FIG. 545B.

FIG. 546 illustrates the embodiment of FIG. 534 applied on the uterine tube of a patient.

FIG. 547 is a schematic sectional view of a mechanically operable non-inflatable constriction device for use in accordance with the invention.

FIGS. 548 and 549 are cross-sectional views taken along the lines XVI-XVI and XVII-XVII, respectively, of FIG. 547.

FIG. 550 schematically shows an alternative design of the embodiment of FIG. 547;

FIG. 551 schematically illustrates a motor arrangement for the design according to FIG. 550;

FIGS. 552 and 553 are schematic sectional views of two alternative designs of non-inflatable constriction devices of the invention.

FIGS. 554 and 555 illustrate a fully open and a reduced constriction opening, respectively, of the embodiment of FIG. 553;

FIG. 556 is a schematic view of a further alternative design of a non-inflatable constriction device of the invention.

FIGS. 557 and 558 illustrate a fully open and a reduced constriction opening, respectively, of the embodiment of FIG. 556;

FIG. 559 is a schematic view of another alternative design of a non-inflatable constriction device of the invention.

FIGS. 560 and 561 are schematic sectional views, respectively, of yet another alternative design of a non-inflatable constriction device of the invention.

FIG. 562A is a schematic view of a hydraulically operable inflatable constriction device for use in accordance with the invention.

FIG. 562B is the same embodiment shown in FIG. 562a with the constriction device inflated.

FIGS. 563A, 563B, 563C and 563D are block diagrams illustrating four different principles for hydraulic operation of the constriction device shown in FIG. 562A.

FIG. 564 is a cross-sectional view of a reservoir having a variable volume controlled by a remote control motor.

FIGS. 565A and 565B are perspective views of a reverse servo in accordance with a particular embodiment of the hydraulic operation principle shown in FIG. 565C.

FIG. 566 is a schematic view of another hydraulically operable constriction device for use in accordance with the invention.

FIG. 567A illustrates the constriction device of FIG. 566 in a constricted state.

FIG. 567B illustrates the constriction device of FIG. 566 in a released state.

FIGS. 568A-568E schematically illustrate different operation stages of an embodiment of the invention, in which a constriction device and a stimulation device co-operate to move the sperms in the uterine tube of a patient's uterine tube.

FIG. 569 is a schematic block diagram illustrating a general embodiment of the apparatus of the invention, in which energy is transferred to energy consuming components of the apparatus implanted in the patient.

FIGS. 570 to 581 are schematic block diagrams illustrating twelve embodiments, respectively, based on the general embodiment shown in FIG. 569, wherein wireless energy is transmitted from outside a patient's body to energy consuming components of the apparatus implanted in the patient.

FIG. 582 illustrates an energy-transforming device in the form of an electrical junction element for use in the apparatus of the present invention.

FIG. 583 is a block diagram illustrating control components of an embodiment of the invention.

FIG. 584 is a schematic view of exemplary circuitry of an embodiment of the invention, in which wireless energy is transformed into a current.

FIGS. 585A-585C schematically illustrate different operation stages of another embodiment of the type shown in FIG. 534, in which a constriction device and a stimulation device co-operate to move the sperms in the uterine tube of a patient's uterine tube.

FIGS. 586A-586B schematically illustrate different operation stages of another embodiment of the type shown in FIGS. 568A-568E, in which a constriction device and a stimulation device co-operate to move the sperms in the uterine tube of a patient's uterine tube.

FIG. 587A is a schematic view of another mechanically operable non-inflatable constriction device for use in accordance with the invention.

FIG. 587B shows the constriction device of FIG. 587A in a constricted state.

FIG. 587C is an end view of the embodiment of FIG. 587B.

FIG. 588 is a schematic block diagram illustrating an arrangement for supplying an accurate amount of wireless energy used for the operation of the constriction/stimulation unit as described above.

FIG. 589 schematically shows an embodiment of the system, in which the apparatus is operated with wire bound energy.

FIG. 590 is a more detailed block diagram of an arrangement for controlling the transmission of wireless energy used for the operation of the constriction/stimulation unit as described above.

FIG. 591 is a circuit for the arrangement shown in FIG. 551, according to a possible implementation example.

FIG. 592 is a sectional view through a constriction device.

FIG. 593A-593C illustrates the constriction device of FIG. 592 in different interrupting stages.

FIG. 594A-594D show a second embodiment of a constriction device.

FIG. 595A is a sectional view of a hip joint;

FIG. 595B is a sectional view of a collum femur;

FIG. 596 is a sectional view of the femoral bone and the hip joint when a bore is created in the femoral bone 5;

FIG. 597 is a sectional view of the femoral bone and the hip joint when a medical device is inserted into the bore in the femoral bone;

FIG. 598 is a sectional view of the femoral bone and the hip joint when the solid lubricant has been pressed into the cavity of the hip joint;

FIG. 599 is a sectional view of the femoral bone and the hip joint when the medical device/cartridge is being removed from the bore in the femoral bone;

FIG. 600 is a sectional view of the femoral bone and the hip joint when a replacement cartridge is placed in the bore;

FIG. 601 shows the creation of a bore in the distal part of the femoral bone;

FIG. 602 shows the placing of a cartridge in the distal part of the femoral bone;

FIG. 603 shows the removal of a cartridge in the distal part of the femoral bone;

FIG. 604 shows the placing of a replacement cartridge in the distal part of the femoral bone;

FIG. 605 shows a prosthetic part having a bore in which the medical device is adapted to be placed;

FIG. 606 shows bore locations in the femoral and tibia bone;

FIG. 607 shows the medical device when placed in a bore in the humerus bone;

FIG. 608 shows the creation of a bore in the pelvic bone from the abdominal side of the pelvic bone;

FIG. 609 shows the placing of a medical device in the bore created in the pelvic bone;

FIG. 610 shows the hip joint in section when the solid lubricant has been pressed into the hip joint cavity;

FIG. 611 shows the removal of the medical device in the direction of the abdominal cavity;

FIG. 612 shows the placing of a replacement cartridge in the bore in the pelvis, from the abdominal side of the pelvic bone;

FIG. 613A shows a sectional view of the medical device/cartridge according to one embodiment;

FIG. 613B shows an alternative embodiment of the retention members;

FIG. 613C shows a sectional view of the medical device/cartridge according to another embodiment;

FIG. 613D shows a sectional view of the medical device/cartridge according to an energized embodiment;

FIG. 613E shows a sectional view of a prosthetic part adapted to replace a portion of the femoral bone;

FIG. 614 is an overall view of a human patient's body showing the position of an implanted assembly according to the invention;

FIG. 615 is a side view of a first embodiment of an implanted assembly according to the invention mounted to a body tissue;

FIG. 616A is a top view of the assembly shown in FIG. 615 having elliptical shape;

FIG. 616B is a top view of the assembly shown in FIG. 615 having circular shape;

FIG. 616C is a sectional view of the assembly shown in FIG. 616B;

FIG. 617 is an overall view of a human patient's body showing an implanted assembly according to the invention connected to an implanted medical device;

FIG. 618 is a block diagram of a control system comprising a control assembly according to the invention;

FIG. 619 is a sectional view of the control assembly shown in FIG. 615;

FIG. 620 is a block diagram showing the different parts of a control assembly according to the invention;

FIG. 621 shows a valve of one embodiment implanted in a human heart, and

FIGS. 622A and 622B show side views of a first embodiment in an open and a closed position, and

FIGS. 623A-623D show side views of a second and a third embodiment in open and a closed position, and

FIGS. 624a-624e show views of a fourth embodiment in various positions, and,

FIGS. 625A and 625B show a valve implanted in a blood vessel, and

FIGS. 626A and 626b show a further embodiment together with a blood vessel, and

FIGS. 627-631 show views of another embodiment of the invention, and

FIGS. 632A-636 show various versions of the invention, and

FIGS. 637A and 637B show a barrier force mechanism of the invention, and

FIGS. 638A-640 show versions of powered movement, and

FIG. 641 show a system of the invention.

FIGS. 642-653 show different embodiments of powered opening and/or closing mechanisms,

FIGS. 654-657 illustrate various methods for implanting the invention in a mammal body.

FIG. 658 shows an embodiment of the implantable constriction device implemented as an anal incontinence treatment apparatus.

FIGS. 659A-C show an embodiment implemented as a constricting device for controlling the fecal passageway of a patient.

FIGS. 660A-B show an embodiment implemented as a constriction device for controlling a flow of sperm through a vas deference of a male patient.

FIGS. 661A-B show an embodiment implemented as a constriction device for constricting a blood vessel, such as a pulmonary artery.

FIG. 662 shows an embodiment implemented as an impotence treatment apparatus for promoting engorgement of the erectile tissue of a male patient.

FIG. 663 shows an embodiment implemented as a hypertension treatment apparatus arranged to extend around a portion of a bile duct of a patient.

FIGS. 664A-B show an embodiment implemented as a constriction device for promoting engorgement of the erectile tissue of a female patient.

FIGS. 665A-B show an embodiment implemented as an aneurysm treatment apparatus.

FIG. 666 illustrates a system for treating a disease, wherein the system includes an apparatus implanted in a patient.

FIGS. 667-681 schematically show various embodiments of the system for wirelessly powering the apparatus of the invention.

FIG. 682 is a schematic block diagram illustrating an arrangement for supplying an accurate amount of energy used for the operation of the apparatus of the invention.

FIG. 683 schematically shows an embodiment of the system, in which the apparatus is operated with wire bound energy.

FIG. 684 is a more detailed block diagram of an arrangement for controlling the transmission of wireless energy used for the operation of the apparatus of the invention.

FIG. 685 is a circuit for the arrangement shown in FIG. 684, according to a possible implementation example.

FIGS. 686-692C show various ways of arranging hydraulic or pneumatic powering of an apparatus implanted in a patient.

FIGS. 693A-693D show embodiments of apparatuses and devices for treating obesity.

FIG. 694A shows a system having a first and a second remote control.

FIG. 694B shows the second remote control comprised in a housing unit.

FIG. 695A schematically shows a medical implant when implanted in a patient.

FIG. 695B shows a flow chart for a method for training a medical implant to recognize a voice command, according to some embodiments;

FIG. 695C shows a flow chart for a method for using voice commands to control a medical implant, according to some embodiments.

FIG. 696 show a schematic illustration of an implantable medical device.

FIGS. 697A-E and illustrates implantable medical devices and external devices for transferring wireless energy to the implantable medical devices.

FIG. 698 illustrates an implantable medical device and an external device configured to transmit data using near field magnetic induction.

FIGS. 699A to L are schematic block diagrams illustrating twelve embodiments, respectively, of the anal incontinence treatment apparatus of the invention, in which wireless energy is transmitted from outside a patient's body to energy consuming components of the apparatus implanted in the patient;

FIG. 699M is a schematic block diagram illustrating conceivable combinations of implanted components for achieving various communication options;

FIG. 699N illustrates an electrical junction element for use in the apparatus of the present invention; and

FIG. 699O illustrates the apparatus in accordance with the invention implanted in a patient.

FIG. 699P is a block diagram illustrating remote control components of an embodiment of the invention, in which wireless energy is transmitted by the use of electromagnetic signals;

FIG. 699Q is a schematic view of exemplary circuitry used for the components of the block diagram of FIG. 699P.

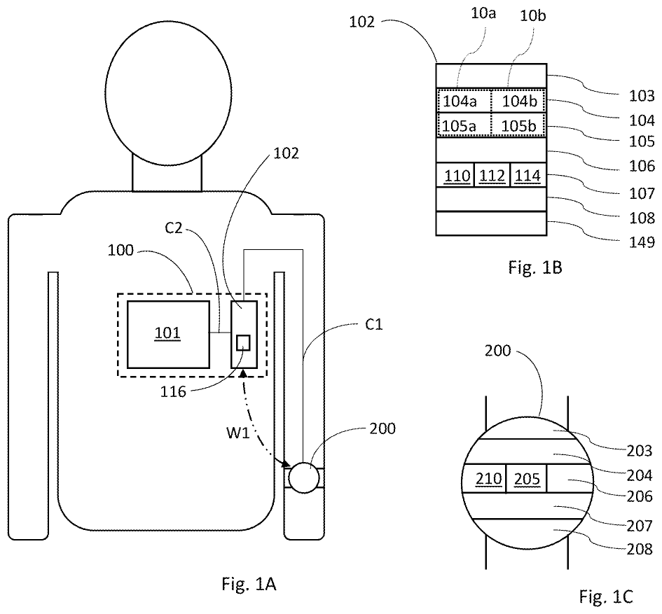

FIG. 700-1A-D are block diagrams of four different principal embodiments of the anal incontinence treatment apparatus according to the invention.

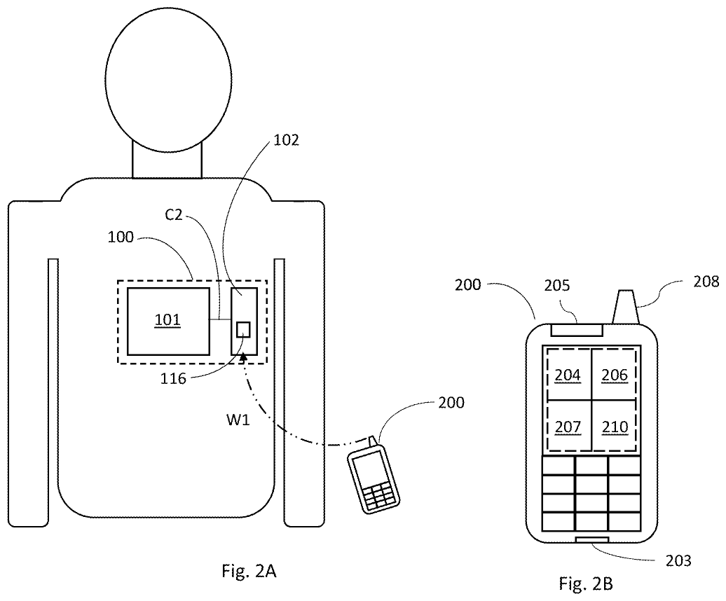

FIG. 700-2A-D are cross-sectional views of a pump mechanism according to FIG. 1C, which is designed to pump fluid in opposite directions by mechanically pushing a wall portion in only one direction.

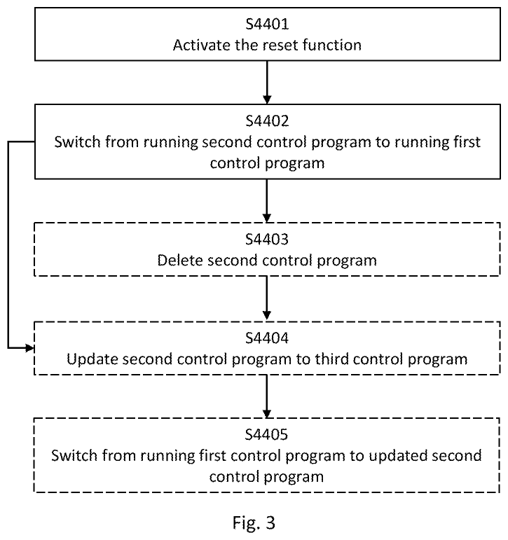

FIG. 700-3 is a cross-sectional view of a reservoir having a variable volume controlled by a remote control motor, in accordance with a particular embodiment of the principal embodiment shown in FIG. 700-1B or 700-2B.

FIG. 700-4 is a cross-sectional view of a reservoir having a variable volume adjustable by manual manipulation, in accordance with a particular embodiment of the principal embodiment shown in FIG. 700-1B or 700-1D.

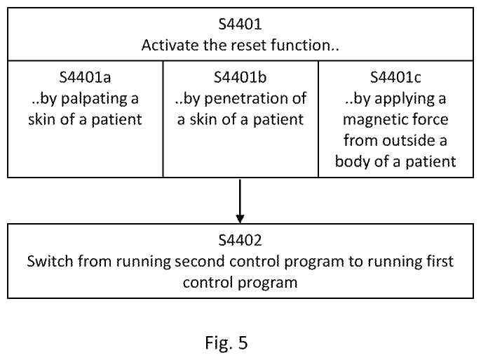

FIG. 700-5A is a perspective view of a hydraulic, pneumatic or mechanical servo system in accordance with a particular embodiment of the principal embodiment shown in FIG. 700-1D.

FIG. 700-5B is a cross-sectional view taken along line VB-VB of FIG. 700-5A.

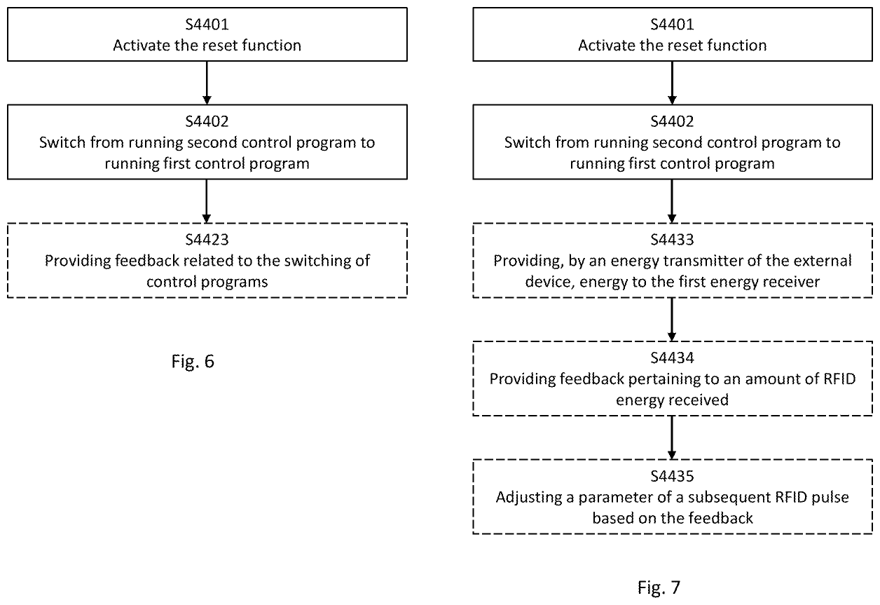

FIG. 700-6 is a block diagram illustrating remote control components of the device of the invention;

FIG. 700-7 is a schematic view of exemplary circuitry used for the block diagram in FIG. 700-4;

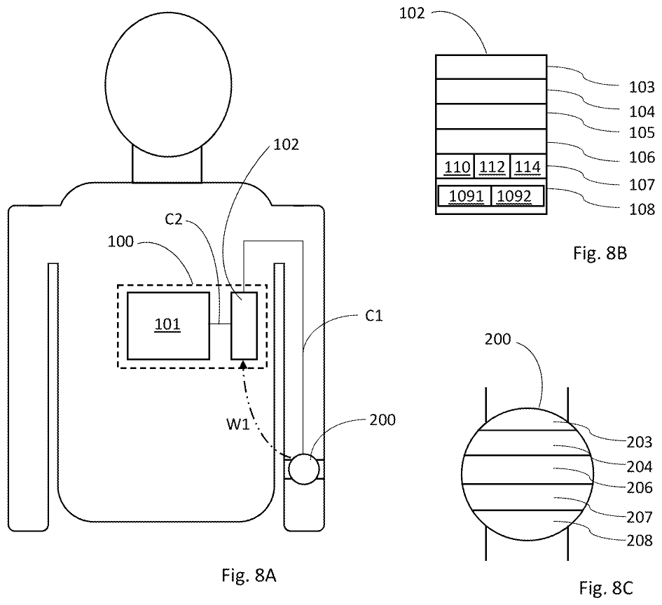

FIG. 700-8 is a schematic view of a band with a cavity defining a restriction opening for use in accordance with the invention.

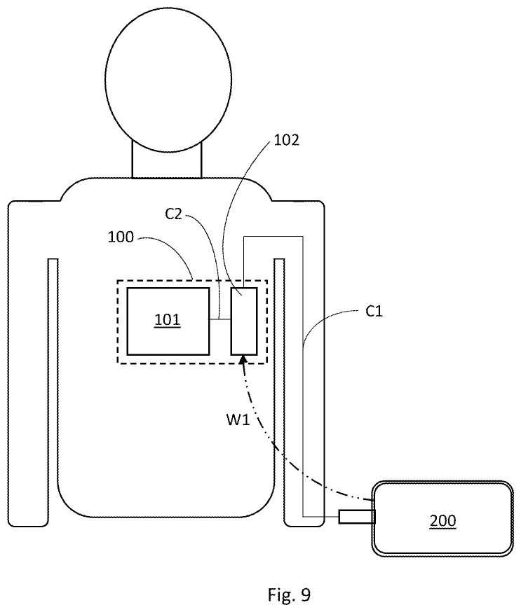

FIGS. 700-9A and 700-9B are schematic views of a first mechanical restriction device for use in accordance with the invention;

FIGS. 700-10A and 700-1OB are schematic views of a second mechanical restriction device for use in accordance with the invention;

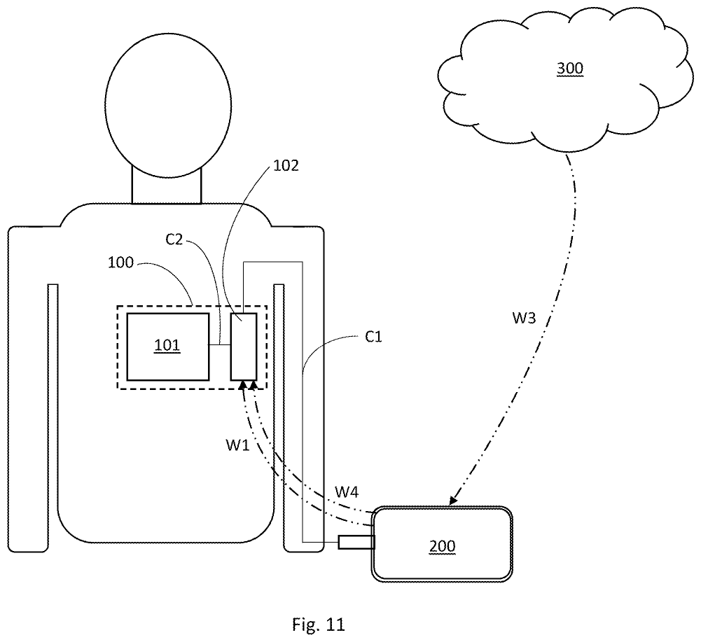

FIG. 700-11 is a schematic view of a third mechanical restriction device for use in accordance with the invention;

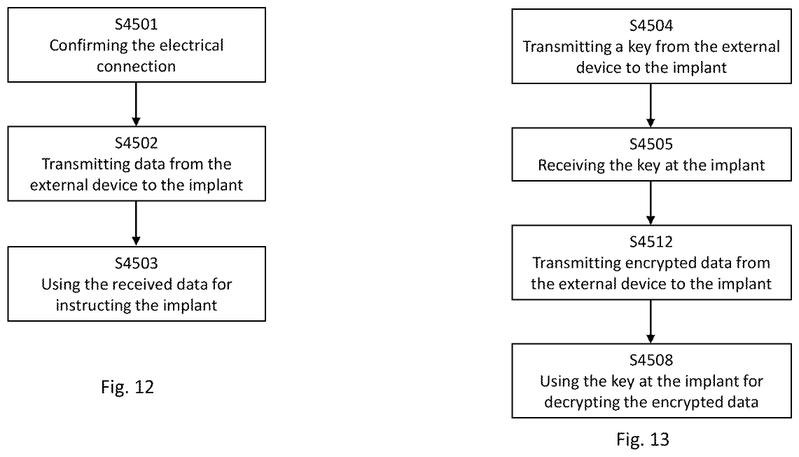

FIG. 700-12A is a schematic front view of a fourth mechanical restriction device for use in accordance with the invention;

FIGS. 700-12B and 700-12C are sectional views along the line A-A of FIG. 700-12A;

FIGS. 700-13A through 700-17B are five modifications of the embodiment of FIGS. 700-12A-700-12C;

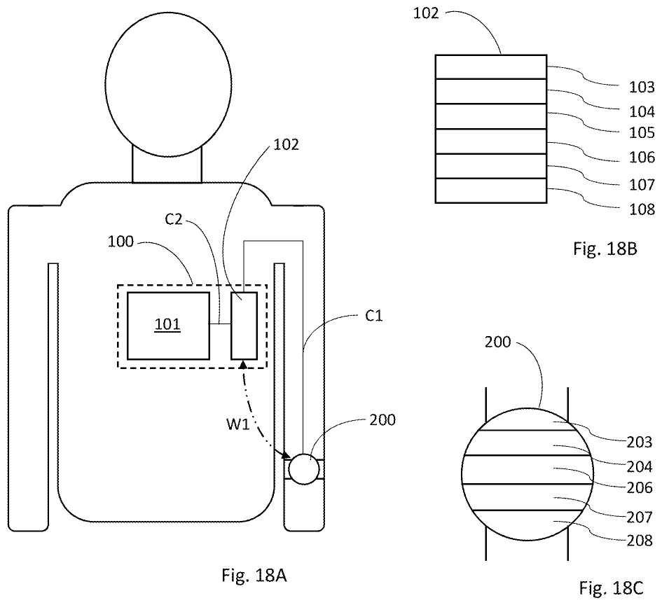

FIG. 700-18 is a view of an inflatable restriction device of the apparatus of the invention: and

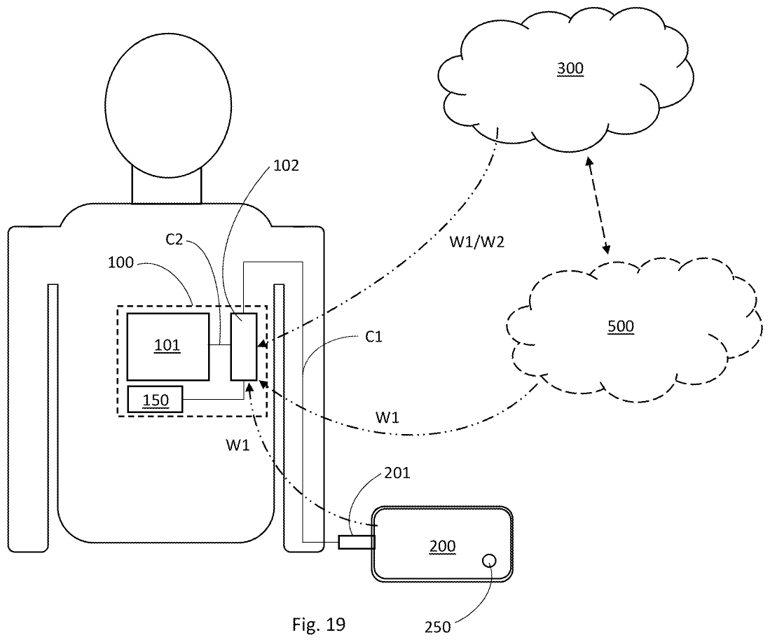

FIG. 700-19 illustrates the apparatus implanted in a patient.

FIGS. 701-1 to 701-6 are schematic block diagrams illustrating six embodiments, respectively, of the invention, in which wireless energy released from an external source of energy is used for direct operation of a restriction device engaging the colon or rectum of a patient;

FIGS. 701-7 to 701-10 are schematic block diagrams illustrating four embodiments, respectively, of the invention, in which energy is released from an implanted source of energy;

FIGS. 701-11 to 701-15 are schematic block diagrams illustrating five embodiments, respectively, of the invention, in which a switch is implanted in the patient for directly or indirectly switching the operation of the restriction device;

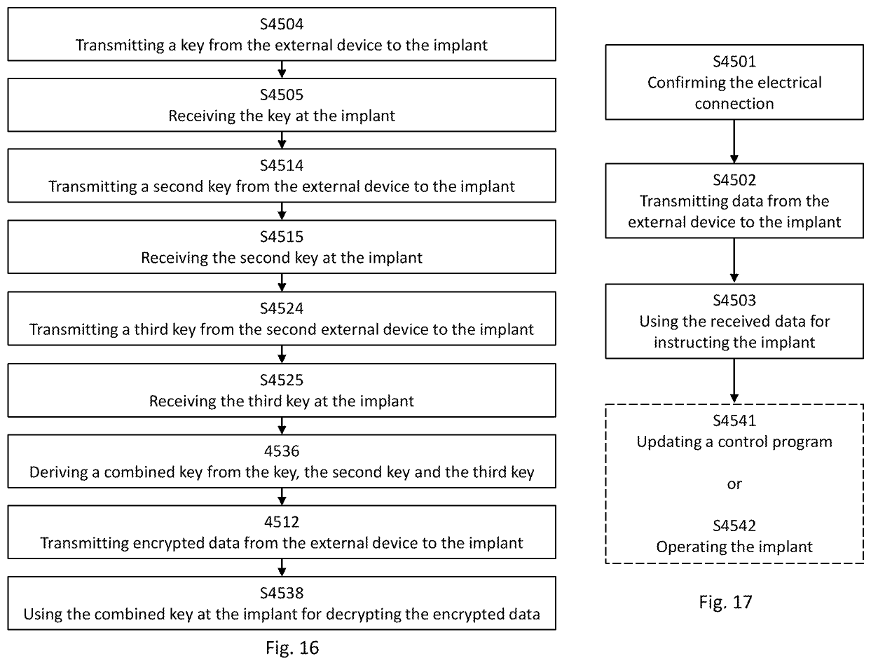

FIGS. 701-16 is a schematic block diagram illustrating conceivable combinations of implantable components for achieving various communication options;

FIGS. 701-17 illustrates the apparatus in accordance with the invention implanted in a patient;

FIGS. 701-18 is a block diagram illustrating remote control components of an embodiment of the invention; and

FIG. 701-19 is a schematic view of exemplary circuitry used for the components of the block diagram of FIG. 701-18.

FIGS. 702-1 to 702-12 are schematic block diagrams illustrating twelve embodiments, respectively, of the male sexual impotence treatment apparatus of the invention, in which wireless energy is transmitted from outside a patient's body to energy consuming components of the apparatus implanted in the patient;

FIG. 702-13 is a schematic block diagram illustrating conceivable combinations of implanted components for achieving various communication options;

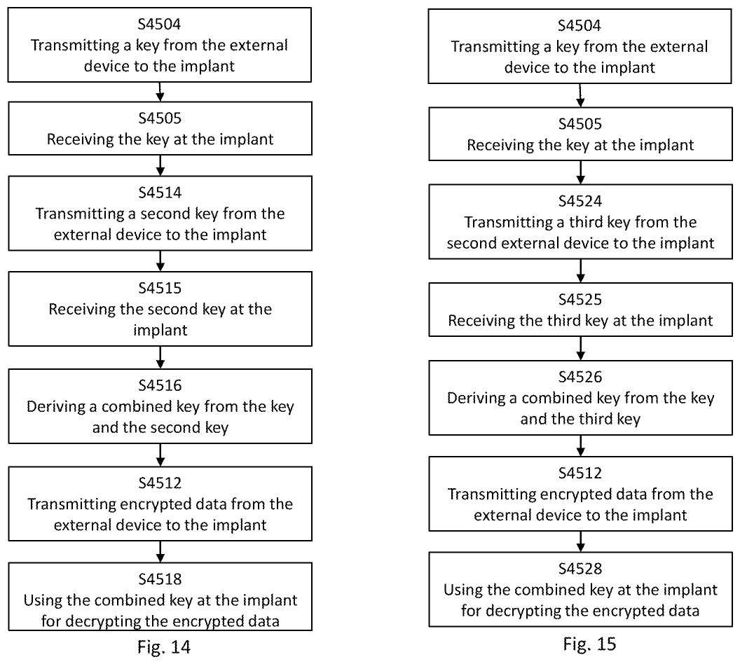

FIG. 702-14 illustrates an electrical junction element for use in the apparatus of the present invention; and FIG. 702-15 illustrates the apparatus in accordance with the invention implanted in a patient.

FIG. 702-16 is a block diagram illustrating remote control components of an embodiment of the invention, in which wireless energy is transmitted by the use of electromagnetic signals; and

FIG. 702-17 is a schematic view of exemplary circuitry used for the components of the block diagram of FIG. 702-16.

FIGS. 703A-703-D are block diagrams of four different principal embodiments of the male sexual impotence treatment apparatus according to the invention.

FIG. 703-2A-703-D are cross-sectional views of a pump mechanism according to FIG. 703-1C, which is designed to pump fluid in opposite directions by mechanically pushing a wall portion in only one direction. FIG. 703-3 is a cross-sectional view of a reservoir having a variable volume controlled by a remote control motor, in accordance with a particular embodiment of the principal embodiment shown in FIG. 703-1B or 703-2B.

FIG. 703-4 is a cross-sectional view of a reservoir having a variable volume adjustable by manual manipulation, in accordance with a particular embodiment of the principal embodiment shown in FIG. 703-1B or 703-1D.

FIG. 703-5A is a perspective view of a hydraulic, pneumatic or mechanical servo system in accordance with a particular embodiment of the principal embodiment shown in FIG. 703-1D.

FIG. 703-5B is a cross-sectional view taken along line VB-VB of FIG. 703-5A.

FIG. 703-6 is a block diagram illustrating remote control components of the device of the invention;

FIG. 703-7 is a schematic view of exemplary circuitry used for the block diagram in FIG. 703-4;

FIG. 703-8 is a schematic view of a band with a cavity defining a restriction opening for use in accordance with the invention.

FIGS. 703-9A and 703-9B are schematic views of a first mechanical restriction device for use in accordance with the invention;

FIGS. 703-10A and 703-1OB are schematic views of a second mechanical restriction device for use in accordance with the invention;

FIG. 703-11 is a schematic view of a third mechanical restriction device for use in accordance with the invention;

FIG. 703-12A is a schematic front view of a fourth mechanical restriction device for use in accordance with the invention;

FIGS. 703-12B and 703-12C are sectional views along the line A-A of FIG. 703-12A;

FIGS. 703-13A through 703-17B are five modifications of the embodiment of FIGS. 703-12A-703-12C;

FIG. 703-18 is a view of an inflatable restriction device of the apparatus of the invention: and

FIG. 703-19 illustrates the apparatus implanted in a patient.

FIG. 704-1 is a schematic sectional view of a preferred first embodiment of the male sexual impotence treatment apparatus in accordance with the invention;

FIGS. 704-2 and 704-3 are cross-sectional views taken along the lines II-II and III-III, respectively, of FIG. 704-1;

FIGS. 704-4 and 704-5 schematically show two alternative designs of the embodiment of

FIG. 704-1;

FIG. 704-6 schematically illustrates a motor arrangement for the design according to FIG. 704-5;

FIG. 704-7 is a schematic sectional view of a second embodiment of the apparatus in accordance with the invention;

FIG. 704-8 schematically illustrates a hydraulic transmission conduit for the embodiment of

FIG. 704-7;

FIG. 704-9 is a schematic sectional view of a third embodiment of the apparatus in accordance with the invention;

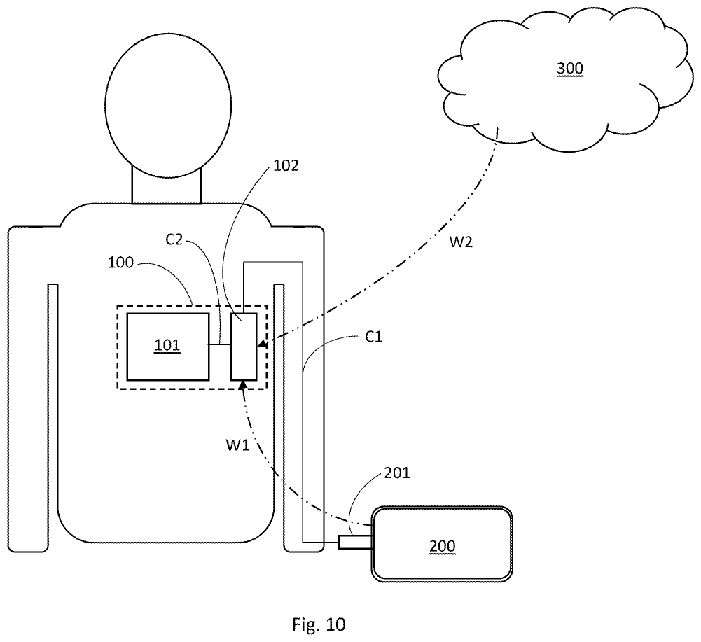

FIG. 704-10 is a modification of the embodiment of FIG. 704-9;

FIG. 704-11 is a schematic view of a fourth embodiment of the apparatus in accordance with the invention;

FIGS. 704-12 and 704-13 are enlarged details of the embodiment of FIG. 704-11;

FIG. 704-14 is a cross-section along the line XIV-XIV of FIG. 704-11;

FIG. 704-15 is a schematic view of a fifth embodiment of the apparatus in accordance with the invention;

FIG. 704-16 is an enlarged detail of FIG. 704-15;

FIG. 704-17 is a cross-section along the line XVII-XVII of FIG. 704-15;

FIGS. 704-18 to 704-21 are schematic sectional views of a sixth, seventh, eighth and ninth embodiments, respectively, of the apparatus in accordance with the invention;

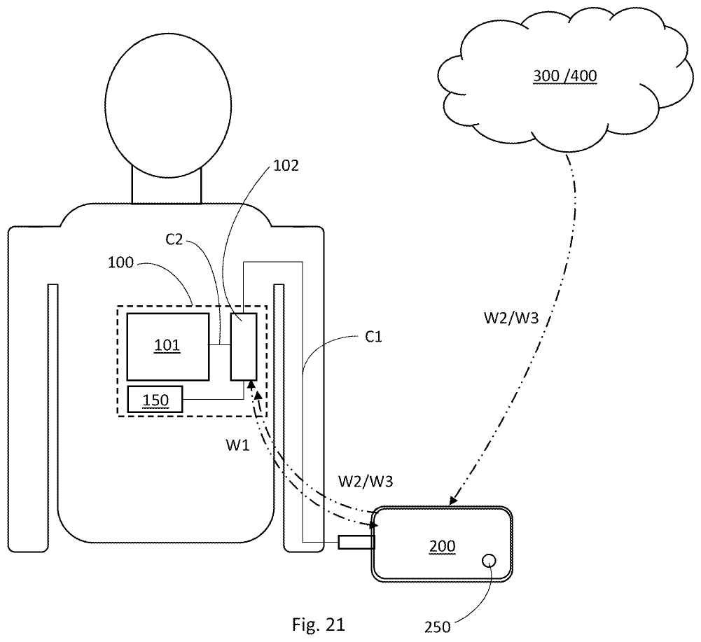

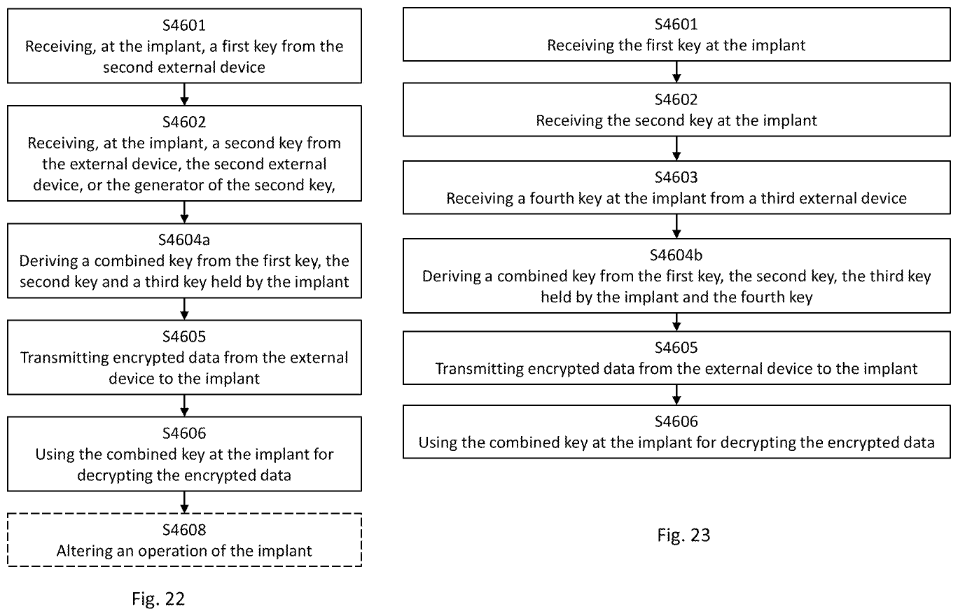

FIGS. 704-22 and 704-23 illustrate a fully open and a reduced restriction opening, respectively, of the embodiment of FIG. 704-21;

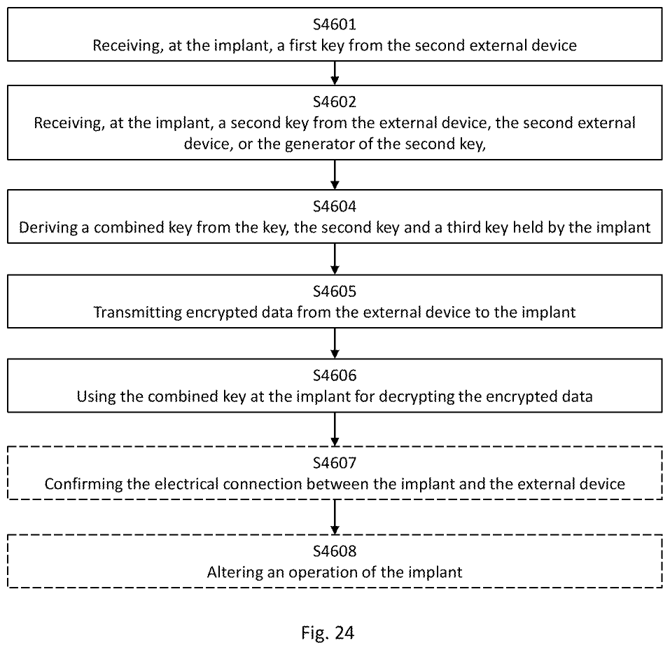

FIG. 704-24 is a schematic view of a tenth embodiment of the apparatus in accordance with the invention;

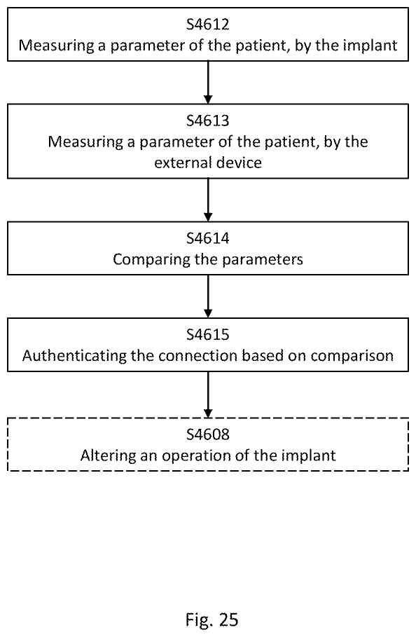

FIG. 704-25 is an enlarged detail of the embodiment of FIG. 704-24;

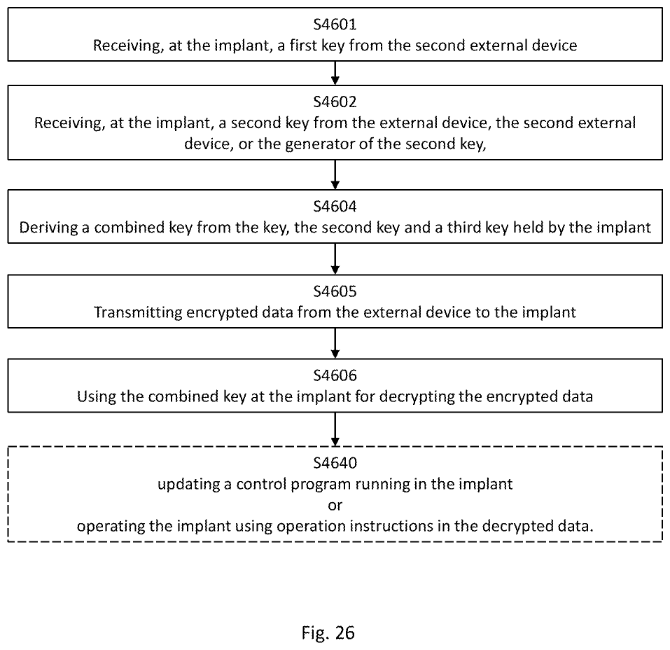

FIGS. 704-26 and 704-27 illustrate a fully open and a reduced restriction opening, respectively, of the embodiment of FIG. 704-24;

FIG. 704-28 schematically illustrates a cushion arrangement for protecting the tissue of the patient;

FIG. 704-29A-D is a block diagram of four different principal embodiments of the invention;

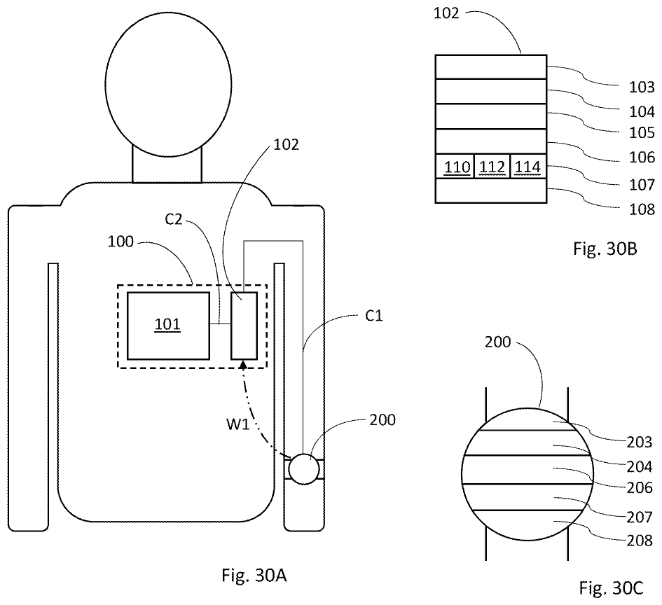

FIG. 704-30A-D are cross-sectional views of a pump mechanism according to FIG. 704-29C, which pumps fluid in opposite directions by mechanically pushing a wall portion in only one direction;

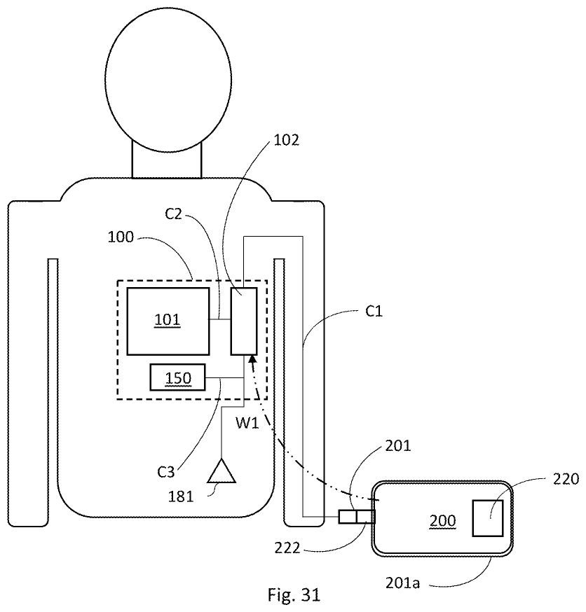

FIG. 704-31 is a cross-sectional view of a reservoir having a variable volume controlled by a remote control motor, in accordance with a particular embodiment of the principal embodiment shown in FIG. 704-29B or 704-30B;

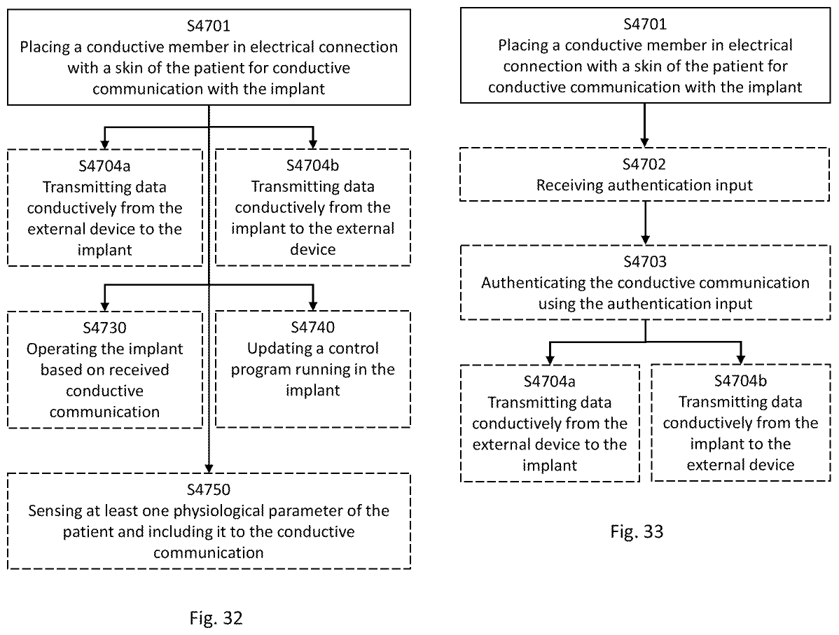

FIG. 704-32 is a cross-sectional view of a reservoir having a variable volume adjustable by manual manipulation, in accordance with a particular embodiment of the principal embodiment shown in FIG. 704-29B or 704-29D;

FIG. 704-33A is a front view of a hydraulic, pneumatic or mechanical reverse servo system in accordance with a particular embodiment of the principal embodiment shown in FIG. 704-29D;

FIG. 704-33B is a cross-sectional view taken along line VB-VB of FIG. 704-33A;

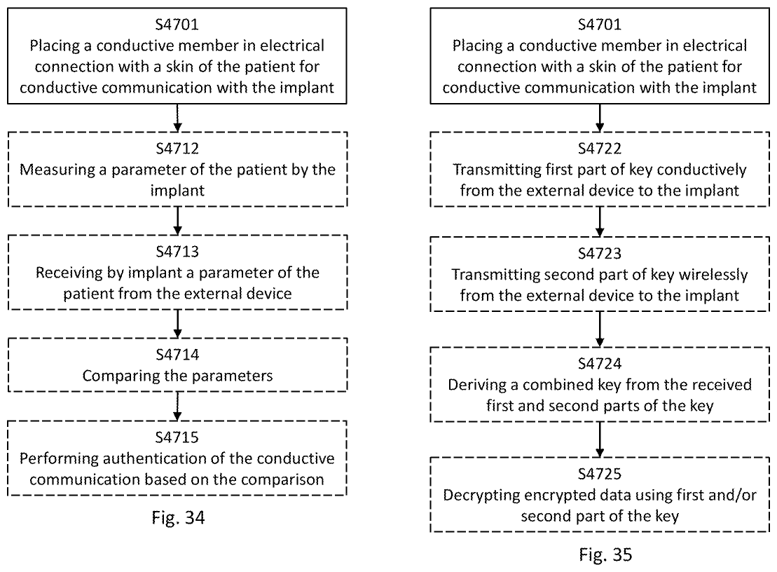

FIG. 704-34 is a block diagram illustrating remote control components of the apparatus of the invention;

FIG. 704-35 is a schematic view of a circuitry used for the system of the block diagram of

FIG. 704-34;

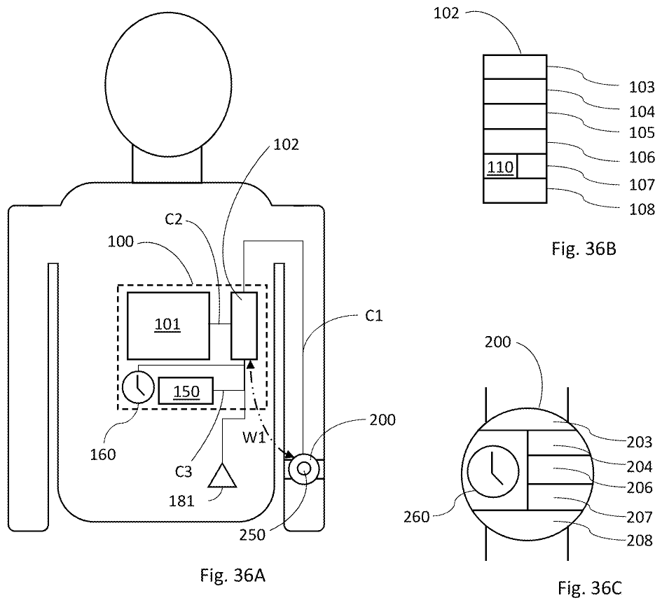

FIGS. 704-36A and 704-36B are schematic views of an eleventh embodiment of the apparatus in accordance with the invention;

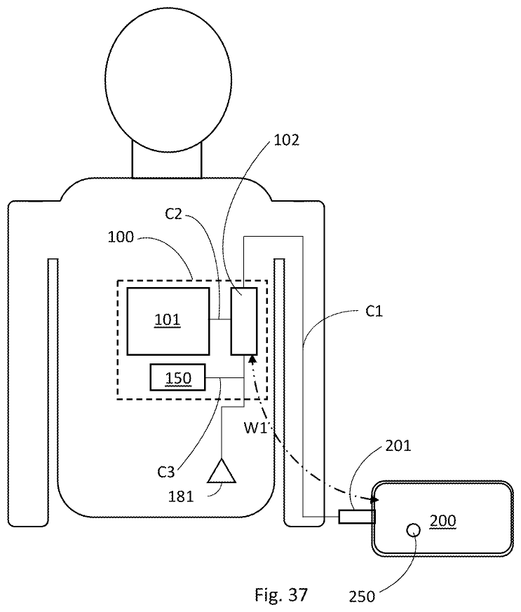

FIGS. 704-37A and 704-37B are schematic views of a twelfth embodiment of the apparatus in accordance with the invention;

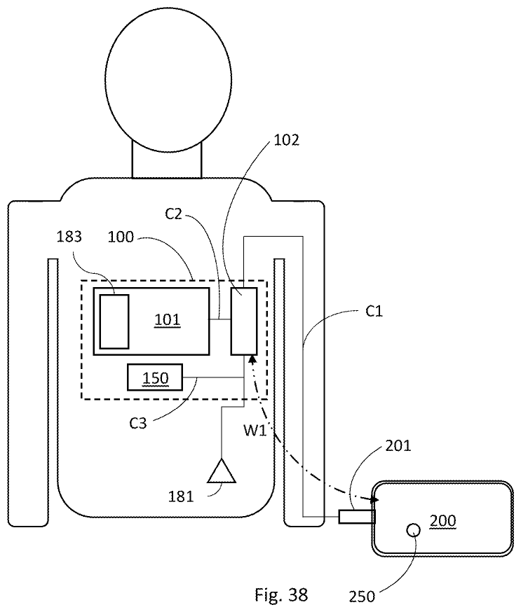

FIG. 704-38 is a schematic view of a thirteenth embodiment of the apparatus in accordance with the invention;

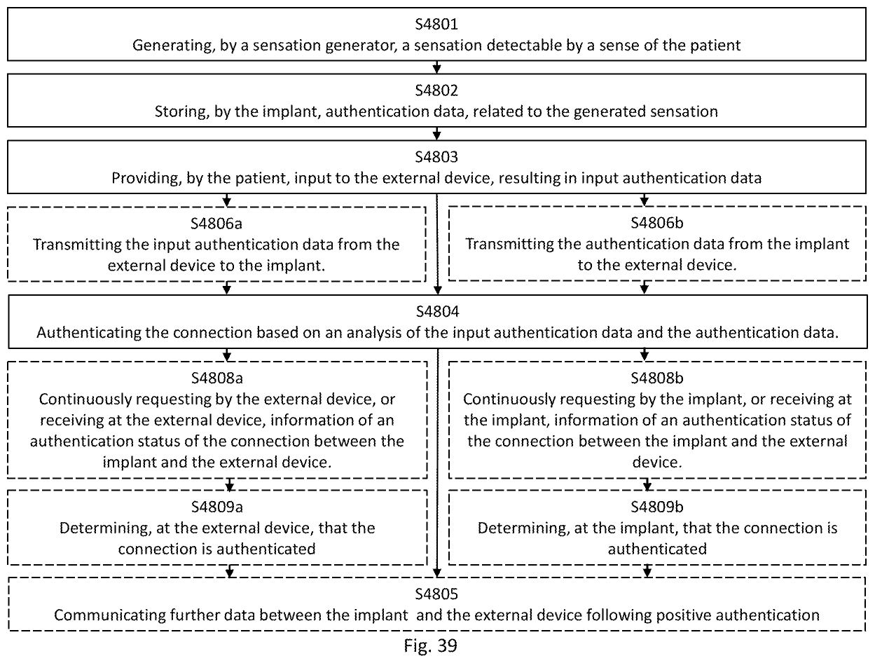

FIGS. 704-39A, 704-39B and 704-39C are a schematic front view and schematic sectional views, respectively, of a fourteenth embodiment of the apparatus in accordance with the invention;

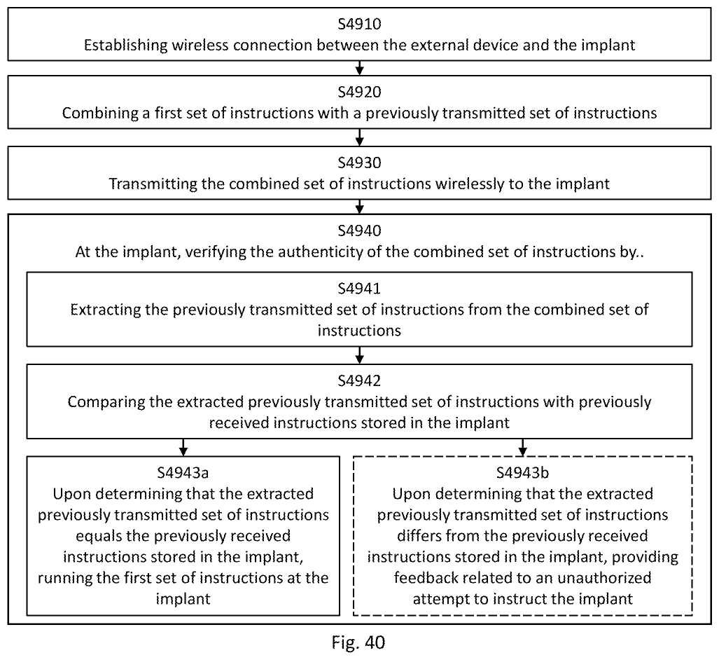

FIGS. 704-40A through 704-44B are five modifications of the embodiment of FIGS. 704-39A-704-39C;

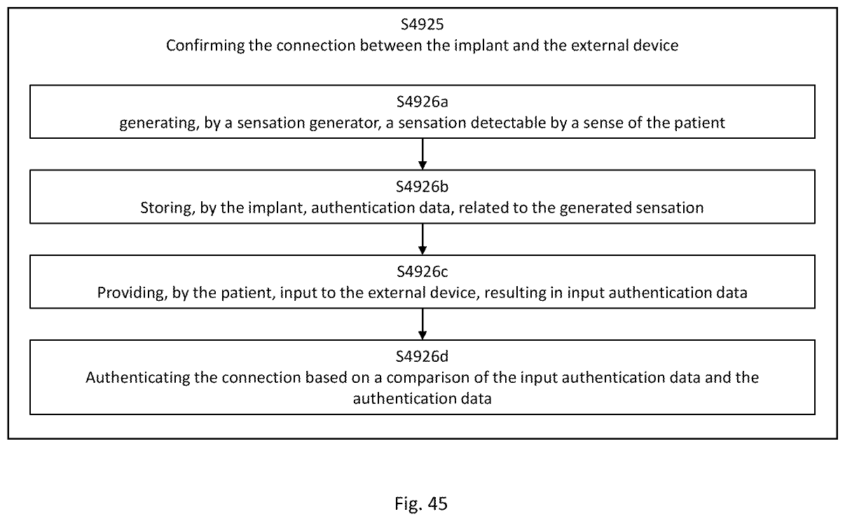

FIG. 704-45 illustrates the apparatus with a restriction device implanted around the corpus cavernosum of a patient; and

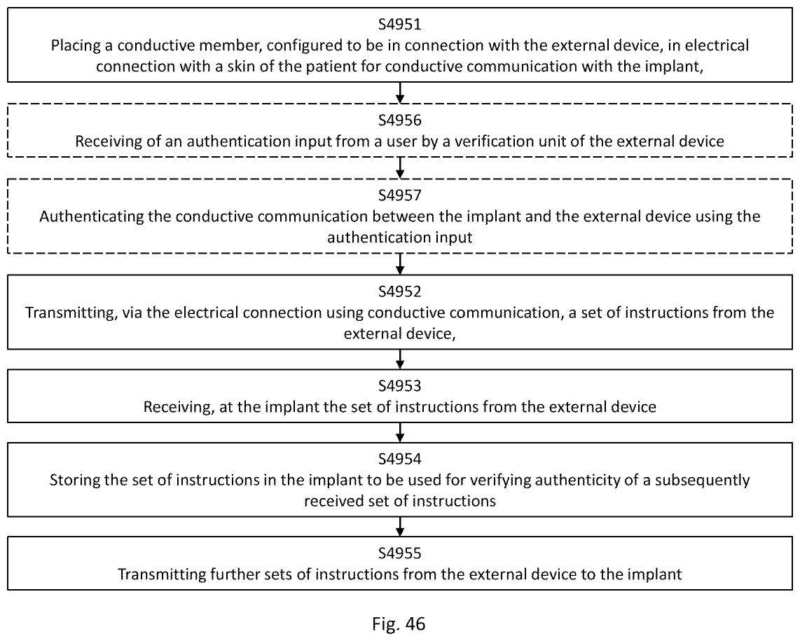

FIG. 704-46 illustrates the apparatus with two restriction members implanted around respective exit veins from the penis of a patient.

FIGS. 705-1 to 705-6 are schematic block diagrams illustrating six embodiments, respectively, of the invention, in which wireless energy released from an external source of energy is used for direct operation of a restriction device engaging the penile tissue of a patient;

FIGS. 705-7 to 705-10 are schematic block diagrams illustrating four embodiments, respectively, of the invention, in which energy is released from an implanted source of energy;

FIGS. 705-11 to 705-15 are schematic block diagrams illustrating five embodiments, respectively, of the invention, in which a switch is implanted in the patient for directly or indirectly switching the operation of the restriction device;

FIG. 705-16 is a schematic block diagram illustrating conceivable combinations of implantable components for achieving various communication options;

FIG. 705-17 illustrates the apparatus in accordance with the invention implanted in a patient;

FIG. 705-18 is a block diagram illustrating remote control components of an embodiment of the invention; and

FIG. 705-19 is a schematic view of exemplary circuitry used for the components of the block diagram of FIG. 705-18.

FIGS. 706-1 to 706-12 are schematic block diagrams illustrating twelve embodiments, respectively, of the urinary incontinence treatment apparatus of the invention, in which wireless energy is transmitted from outside a patient's body to energy consuming components of the apparatus implanted in the patient.

FIG. 706-13 is a schematic block diagram illustrating conceivable combinations of implanted components for achieving various communication options;

FIG. 706-14 illustrates an electrical junction element for use in the apparatus of the present invention; and

FIG. 706-15 illustrates the apparatus in accordance with the invention implanted in a patient;

FIG. 706-16 is a block diagram illustrating remote control components of an embodiment of the invention, in which wireless energy is transmitted by the use of electromagnetic signals; and

FIG. 706-17 is a schematic view of exemplary circuitry used for the components of the block diagram of FIG. 706-16.

FIGS. 707-1A-D are block diagrams of four different principal embodiments of the urinary incontinence treatment apparatus according to the invention.

FIG. 707-2A-D are cross-sectional views of a pump mechanism according to FIG. 707-1C, which is designed to pump fluid in opposite directions by mechanically pushing a wall portion in only one direction.

FIG. 707-3 is a cross-sectional view of a reservoir having a variable volume controlled by a remote control motor, in accordance with a particular embodiment of the principal embodiment shown in FIG. 707-1B or 707-2B.

FIG. 707-4 is a cross-sectional view of a reservoir having a variable volume adjustable by manual manipulation, in accordance with a particular embodiment of the principal embodiment shown in FIG. 707-1B or 707-1D.

FIG. 707-5A is a perspective view of a hydraulic, pneumatic or mechanical servo system in accordance with a particular embodiment of the principal embodiment shown in FIG. 707-1D.

FIG. 707-5B is a cross-sectional view taken along line VB-VB of FIG. 707-5A.

FIG. 707-6 is a block diagram illustrating remote control components of the device of the invention;

FIG. 707-7 is a schematic view of exemplary circuitry used for the block diagram in FIG. 707-4;

FIG. 707-8 is a schematic view of a band with a cavity defining a restriction opening for use in accordance with the invention.

FIGS. 707-9A and 707-9B are schematic views of a first mechanical restriction device for use in accordance with the invention;

FIGS. 707-10A and 707-1OB are schematic views of a second mechanical restriction device for use in accordance with the invention;

FIG. 707-11 is a schematic view of a third mechanical restriction device for use in accordance with the invention;

FIG. 707-12A is a schematic front view of a fourth mechanical restriction device for use in accordance with the invention;

FIGS. 707-12B and 707-12C are sectional views along the line A-A of FIG. 707-12A;

FIGS. 707-13A through 707-17B are five modifications of the embodiment of FIGS. 707-12A-707-12C;

FIG. 707-18 is a view of an inflatable restriction device of the apparatus of the invention: and

FIG. 707-19 illustrates the apparatus implanted in a patient.

FIG. 708-1 is a schematic sectional view of a preferred first embodiment of the urinary incontinence treatment apparatus in accordance with the invention;

FIGS. 708-2 and 708-3 are cross-sectional views taken along the lines II-II and III-III, respectively, of FIG. 708-1;

FIGS. 708-4 and 708-5 schematically show two alternative designs of the embodiment of

FIG. 708-1;

FIG. 708-6 schematically illustrates a motor arrangement for the design according to FIG. 708-5;

FIG. 708-7 is a schematic sectional view of a second embodiment of the apparatus in accordance with the invention;

FIG. 708-8 schematically illustrates a hydraulic transmission conduit for the embodiment of

FIG. 708-7;

FIG. 708-9 is a schematic sectional view of a third embodiment of the apparatus in accordance with the invention;

FIG. 708-10 is a modification of the embodiment of FIG. 708-9;

FIG. 708-11 is a schematic view of a fourth embodiment of the apparatus in accordance with the invention;

FIGS. 708-12 and 708-13 are enlarged details of the embodiment of FIG. 708-11;

FIG. 708-14 is a cross-section along the line XIV-XIV of FIG. 708-11;

FIG. 708-15 is a schematic view of a fifth embodiment of the apparatus in accordance with the invention;

FIG. 708-16 is an enlarged detail of FIG. 708-15;

FIG. 708-17 is a cross-section along the line XVII-XVII of FIG. 708-15;

FIGS. 708-18 to 708-21 are schematic sectional views of a sixth, seventh, eighth and ninth embodiments, respectively, of the apparatus in accordance with the invention;

FIGS. 708-22 and 708-23 illustrate a fully open and a reduced restriction opening, respectively, of the embodiment of FIG. 708-21;

FIG. 708-24 is a schematic view of a tenth embodiment of the apparatus in accordance with the invention;

FIG. 708-25 is an enlarged detail of the embodiment of FIG. 708-24;

FIGS. 708-26 and 708-27 illustrate a fully open and a reduced restriction opening, respectively, of the embodiment of FIG. 708-24;

FIG. 708-28 schematically illustrates a cushion arrangement for protecting the tissue of the patient;

FIG. 708-29A-D is a block diagram of four different principal embodiments of the invention;

FIG. 708-30A-D are cross-sectional views of a pump mechanism according to FIG. 708-29C, which pumps fluid in opposite directions by mechanically pushing a wall portion in only one direction;

FIG. 708-31 is a cross-sectional view of a reservoir having a variable volume controlled by a remote control motor, in accordance with a particular embodiment of the principal embodiment shown in FIG. 708-29B or 708-30B;

FIG. 708-32 is a cross-sectional view of a reservoir having a variable volume adjustable by manual manipulation, in accordance with a particular embodiment of the principal embodiment shown in FIG. 708-29B or 708-29D;

FIG. 708-33A is a front view of a hydraulic, pneumatic or mechanical reverse servo system in accordance with a particular embodiment of the principal embodiment shown in FIG. 708-29D;

FIG. 708-33B is a cross-sectional view taken along line VB-VB of FIG. 708-33A;

FIG. 708-34 is a block diagram illustrating remote control components of the apparatus of the invention;

FIG. 708-35 is a schematic view of a circuitry used for the system of the block diagram of FIG. 708-34;

FIGS. 708-36A and 708-36B are schematic views of an eleventh embodiment of the apparatus in accordance with the invention;

FIGS. 708-37A and 708-37B are schematic views of a twelfth embodiment of the apparatus in accordance with the invention;

FIG. 708-38 is a schematic view of a thirteenth embodiment of the apparatus in accordance with the invention;

FIGS. 708-39A, 708-39B and 708-39C are a schematic front view and schematic sectional-views, respectively, of a fourteenth embodiment of the apparatus in accordance with the invention;

FIGS. 708-40A through 708-44B are five modifications of the embodiment of FIGS. 708-39A-708-39C;

FIG. 708-45 illustrates the apparatus with an implanted restriction device engaging the urethra.

FIGS. 709-1 to 709-6 are schematic block diagrams illustrating six embodiments, respectively, of the invention, in which wireless energy released from an external source of energy is used for direct operation of a restriction device engaging the urethra or urine bladder of a patient;

FIGS. 709-7 to 709-10 are schematic block diagrams illustrating four embodiments, respectively, of the invention, in which energy is released from an implanted source of energy;

FIGS. 709-11 to 709-15 are schematic block diagrams illustrating five embodiments, respectively, of the invention, in which a switch is implanted in the patient for directly or indirectly switching the operation of the restriction device;

FIG. 709-16 is a schematic block diagram illustrating conceivable combinations of implantable components for achieving various communication options;

FIG. 709-17 illustrates the apparatus in accordance with the invention implanted in a patient;

FIG. 709-18 is a block diagram illustrating remote control components of an embodiment of the invention; and

FIG. 709-19 is a schematic view of exemplary circuitry used for the components of the block diagram of FIG. 709-18.

FIGS. 710-1A to 710-1F schematically an apparatus according to the invention implanted in a human.

FIG. 710-1G illustrates a system for treating a disease, wherein the system includes an apparatus of any of FIG. 710-1A to 710-1F implanted in a patient.

FIGS. 710-2-710-16 schematically show various embodiments of the system for wirelessly powering the apparatus shown in FIGS. 710-1A-710-1F.

FIG. 710-17 is a schematic block diagram illustrating an arrangement for supplying an accurate amount of energy used for the operation of the apparatus shown in FIG. 710-2.

FIG. 710-18 schematically shows an embodiment of the system, in which the apparatus is operated with wire bound energy.

FIG. 710-19 is a more detailed block diagram of an arrangement for controlling the transmission of wireless energy used for the operation of the apparatus shown in FIG. 710-2.

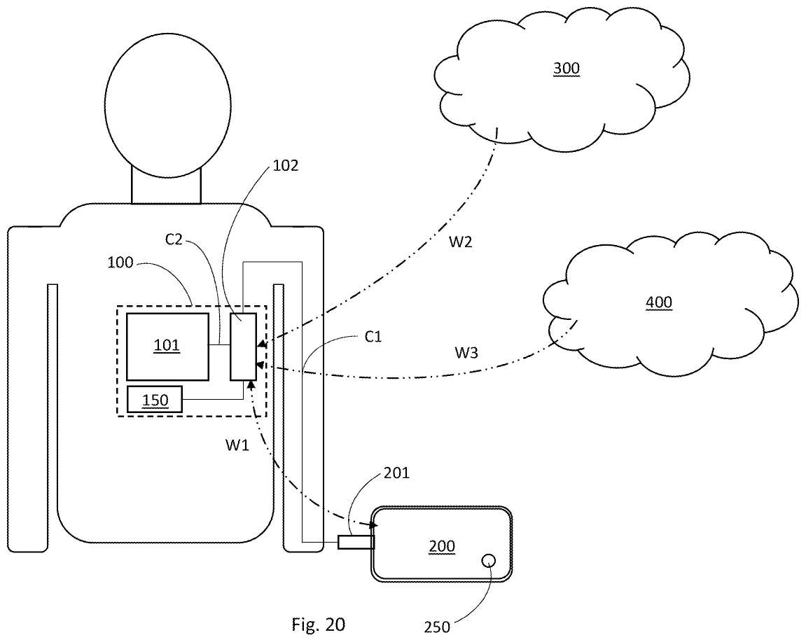

FIG. 710-20 is a circuit for the arrangement shown in FIG. 710-19, according to a possible implementation example.

FIGS. 710-21-710-27 show various ways of arranging hydraulic or pneumatic powering of an apparatus implanted in a patient.

FIG. 711-1 is a schematic sectional view of a preferred first embodiment of the anal incontinence treatment apparatus in accordance with the invention;

FIGS. 711-2 and 711-3 are cross-sectional views taken along the lines II-II and III-III, respectively, of FIG. 711-1;

FIGS. 711-4 and 711-5 schematically show two alternative designs of the embodiment of FIG. 711-1;

FIG. 711-6 schematically illustrates a motor arrangement for the design according to FIG. 711-5;

FIG. 711-7 is a schematic sectional view of a second embodiment of the apparatus in accordance with the invention;

FIG. 711-8 schematically illustrates a hydraulic transmision conduit for the embodiment of FIG. 711-7;

FIG. 711-9 is a schematic sectional view of a third embodiment of the apparatus in accordance with the invention;

FIG. 711-10 is a modification of the embodiment of FIG. 711-9;

FIG. 711-11 is a schematic view of a fourth embodiment of the apparatus in accordance with the invention;

FIGS. 711-12 and 711-13 are enlarged details of the embodiment of FIG. 711-11;

FIG. 711-14 is a cross-section along the line XIV-XIV of FIG. 711-11;

FIG. 711-15 is a schematic view of a fifth embodiment of the apparatus in accordance with the invention;

FIG. 711-16 is an enlarged detail of FIG. 711-15;

FIG. 711-17 is a cross-section along the line XVII-XVII of FIG. 711-15;

FIGS. 711-18 to 711-21 are schematic sectional views of a sixth, seventh, eighth and ninth embodiments, respectively, of the apparatus in accordance with the invention;

FIGS. 711-22 and 711-23 illustrate a fully open and a reduced restriction opening, respectively, of the embodiment of FIG. 711-21;

FIG. 711-24 is a schematic view of a tenth embodiment of the apparatus in accordance with the invention;

FIG. 711-25 is an enlarged detail of the embodiment of FIG. 711-24;

FIGS. 711-26 and 711-27 illustrate a fully open and a reduced restriction opening, respectively, of the embodiment of FIG. 711-24;

FIG. 711-28 schematically illustrates a cushion arrangement for protecting the tissue of the patient;

FIG. 711-29A-D is a block diagram of four different principal embodiments of the invention;

FIG. 711-30A-D are cross-sectional views of a pump mechanism according to FIG. 711-29C, which pumps fluid in opposite directions by mechanically pushing a wall portion in only one direction;

FIG. 711-31 is a cross-sectional view of a reservoir having a variable volume controlled by a remote control motor, in accordance with a particular embodiment of the principal embodiment shown in FIG. 711-29B or 711-30B;

FIG. 711-32 is a cross-sectional view of a reservoir having a variable volume adjustable by manual manipulation, in accordance with a particular embodiment of the principal embodiment shown in FIG. 711-29B or 711-29D;

FIG. 711-33A is a front view of a hydraulic, pneumatic or mechanical reverse servo system in accordance with a particular embodiment of the principal embodiment shown in FIG. 711-29D;

FIG. 711-33B is a cross-sectional view taken along line VB-VB of FIG. 711-33A;

FIG. 711-34 is a block diagram illustrating remote control components of the apparatus of the invention;

FIG. 711-35 is a schematic view of a circuitry used for the system of the block diagram of FIG. 711-34;

FIGS. 711-36A and 711-36B are schematic views of an eleventh embodiment of the apparatus in accordance with the invention;

FIGS. 711-37A and 711-37B are schematic views of a twelfth embodiment of the apparatus in accordance with the invention;

FIG. 711-38 is a schematic view of a thirteenth embodiment of the apparatus in accordance with the invention;

FIGS. 711-39A, 711-39B and 711-39C are a schematic front view and schematic sectional views, respectively, of a fourteenth embodiment of the apparatus in accordance with the invention;

FIGS. 711-40A through 711-44B are five modifications of the embodiment of FIGS. 711-39A-711-39C;

FIG. 711-45 illustrates the apparatus with a restriction device implanted around the colon.

### DETAILED DESCRIPTION OF EMBODIMENTS

In the following, embodiments will be described in conjunction with a plurality of drawings. To simplify for the reader, here follows a general description of features used when exemplifying the different embodiments. It should be noted however, that the invention is defined by the independent claims. It should be noted that in the drawings, the same reference number is used throughout the drawings for defining the same feature. Consequently, if a reference number in a particular drawing is not explained or defined in the description relating to that particular drawing, any other part of the text which define that particular reference number may be used for explaining the feature in the context of the particular drawing. It should also be noted that all features in the drawings which are not described in conjunction with a particular drawing should be considered optional unless stated otherwise.

### General Definition of Features Used in this Disclosure

An implant, or operable implant, is to be understood as any implant that could be operated for performing a function in relation to the body of the patient when implanted in the patient. A medical device adapted for implantation in a body is also to be understood as an implant. The patient may also be called user, person, or be referred to as a “body” or mammal. In the following, the terms “medical implant”, “implantable medical device”, “medica device”, “implant” and “operable implant” may be used interchangeably.

In the drawings, the implant is generally illustrated as being placed in the abdominal area of the patient. It could, however, equally be placed in other parts of the patient's body.

To be operated includes the altering of the size and/or shape of a portion of the implant, delivering an active or inactive substance to the body of the patient, electrically stimulating a portion of the body of the patient, sensing a physical or functional parameter of the operable implant and/or a physiological or physical parameter of the patient, communicating with an external unit on the outside of the skin of the patient and receiving or transmitting energy at the operable implant, from an external unit. An operable implant could for example be a pacemaker unit, an implantable cardioverter defibrillator, an external heart compression device, an apparatus assisting the pump function of the heart, such as an LVAD device, an operable artificial heart valve, an implantable drug delivery device, such as an implantable device for delivering insulin or chemotherapeutic agents, a hydraulic, mechanic and/or electric constriction implant for constricting for example: an intestine for treating anal incontinence, an intestine for handling a stoma, the urethra for treating urinary incontinence, the bile duct for treating gall bladder malfunction, an oviduct for purpose of fertility control, the vas deference for the purpose of potency control, a blood vessel for purpose of increasing the blood volume in an erectile tissue, or for the purpose of constricting or restraining an aneurysm. An operable implant may further be an operable implant for treating obesity, such as an operable volume filling device for reducing the volume of the stomach, an operable gastric band for limiting the food passageway, or an operable implant for stretching the stomach wall for creating a feeling of satiety. The operable implant may be an operable device for treating GERD an operable cosmetic implant, such as an operable breast augmentation implant, or an implant for adjusting or replacing any bone part of the body. Furthermore, the implant could be replacing an organ or part of an organ, or the function thereof could be adjusted or replaced. Other examples of implants are implants treating impotence by implanted drug delivery, implants affecting blood flow, vascular treatment devices which may include blood clot removal, implants affecting fertility and/or infertility, or implants adapted to move fluid inside the body. The above listed examples of an operable implant are to be seen as examples not in any way limiting the possible application areas of the operable implant.

Body engaging portion is to be understood as any part or portion of the operable implant that is directly or indirectly connected to the body of the patient for performing a function in relation to the body of the patient. The function could for example be pressing and/or pulling against a portion of the body of the patient, delivering a substance to the body of the patient, collecting a sample from the body of the patient, electrically stimulating a portion of the body of the patient and/or filling or emptying an implantable volume filling device with a hydraulic fluid. The body engaging portion may alternatively be referred to as the active unit or the active device of the implant.

A physical or functional parameter of the operable implant could for example be an electrical parameter, such as voltage, current or impedance, a parameter related to a fluid, such as pressure, flow rate, temperature, volume, weight, or viscosity. The parameter could be related to energy received at the operable implant, energy delivered to the body of the patient, fluid received at the operable implant, fluid delivered to the body of the patient, force exerted on the body of the patient or time elapsed since an action was performed in relation to the body of the patient. These physical or functional parameters can be measured or sensed by means of sensor(s), further described herein with reference to aspect 255SE. In such cases, the implant comprises the necessary sensor(s) needed to perform such sensing/measurement(s).

A physiological or physical parameter of the patient could for example be the blood pressure of the patient, a blood flow, a parameter related to blood saturation, a parameter related to an ischemia marker, a temperature of the body of the patient, a parameter related to muscle activity or a parameter related to the activity of the gastro-intestinal system. These physiological or physical parameters can be measured or sensed by means of sensor(s), further described herein with reference to aspect 255SE. In such cases, the implant comprises the necessary sensor(s) needed to perform such sensing/measurement(s).

The operation device in the operable implant may comprise an electrical motor for transforming electrical energy into mechanical work. The electrical motor could for example be an alternating current (AC) electrical motor, such as a three-phase electrical motor (which may be controlled using variable-frequency drive), a direct current (DC) electrical motor, a linear electrical motor, an AC or DC axial electrical motor, a piezo-electric motor, a bimetal motor, or a memory metal motor.

Alternatively, other types of motors may be used such as a hydraulic motor, a pneumatic motor, or a thermodynamic motor such as a Stirling engine.

As an alternative to a motor, an actuator may perform the required mechanical work within in the operable implant. Compared to a motor, an actuator generally only provides work between end points within a limited rotational range and does generally not provide full rotations to a drive shaft like a motor. The actuator may be electrically powered and controlled in same or similar ways as the electrical motor described in the above. An actuator may also be hydraulic, pneumatic, or thermodynamically based.

Generally, a medical system including an operable implant comprising an implantable body engaging portion and an implantable operation device, and components thereof, is described herein. The implantable operation device could be adapted to electrically, mechanically or hydraulically operate the body engaging portion and could be powered by means of wireless energy transfer from the outside of the body of the patient, or by means of an implantable battery adapted to store electrical energy in the body of the patient. The operation device may comprise an electrical motor for transferring electrical energy to mechanical work (force*distance) and the electrical motor may be connected to one or more gear systems for altering the velocity and/or force/torque and/or direction of the supplied force. The operable implant may additionally comprise a communications unit for communicating with portions of the operable implant, other operable implants and/or external units. The communication with the external unit could comprise control signals from the external unit for controlling the operable implant or could comprise feedback signals from the operable implant, which for example could be sensor parameters such as physiological or physical sensor parameters related to the status of the body of the patient, or physical or functional parameters related to status of the operable implant.

The implant may comprise a communication unit. The unit may alternatively be called the internal communication unit or the communication unit of the implant. Alternatively, the communication unit may be called a controller. The communication unit may comprise a collection of communication related sub-units such as a wired transceiver, a wireless transceiver, energy storage, an energy receiver, a computing unit, a memory, or a feedback unit. The sub-units of the communication unit may cooperate with each other or operate independently with different purposes. The sub-units of the internal communication unit may inherit the prefix “internal”. This is to distinguish these sub-units from the sub-units of the external communication unit as similar sub-units may be present for both communication units. The sub-units of the external communication unit may similarly inherit the prefix “external”.

A wireless transceiver may comprise both a wireless transmitter and a wireless receiver. The wireless transceiver may also comprise a first wireless transceiver and a second wireless transceiver. In this case, the wireless transceiver may be part of a first communication system (using the first wireless transceiver) and a second communication system (using the second wireless transceiver).

In some embodiments, two communication systems may be implemented using a single wireless transceiver in e.g. the implant and a single wireless transceiver in e.g. an external device (i.e. one antenna at the implant and one antenna at the external device), but where for example the network protocol used for data transmission from the external device to the implant is different from the network protocol used for data transmission from the implant to the external device, thus achieving two separate communication systems.

Alternatively, the wireless transceiver may be referred to as either a wireless transmitter or a wireless receiver as not all embodiments of secure wireless communication discussed herein require two-way communication capability of the wireless transceiver. The wireless transceiver may transmit or receive wireless communication via wireless connections. The wireless transceiver may connect to both the implant and to external devices, i.e. devices not implanted in the patient.

The wireless connections may be based on radio frequency identification (RFID), near field charge (NFC), Bluetooth, Bluetooth low energy (BLE), or wireless local area network (WLAN). The wireless connections may further be based on mobile telecommunication regimes such as 1G, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

A wired transceiver may comprise both a wired transmitter and a wired receiver. The wording wired transceiver aims to distinguish between it and the wireless transceiver. It may generally be considered a conductive transceiver. The wired transceiver may transmit or receive conductive communication via conductive connections. Conductive connections may alternatively be referred to as electrical connections or as wired connections. The wording wired however, does not imply there needs to be a physical wire for conducting the communication. The body tissue of the patient may be considered as the wire. Conductive connection may use the body of the patient as a conductor.

Conductive connections may still use ohmic conductors such as metals to at least some extent, and more specifically at the interface between the wired transceiver and the chosen conductor.

Communication, conductive or wireless may be understood as digital or analogue. In analogue communication, the message signal is in analogue form i.e., a continuous time signal. In digital communication, usually digital data i.e., discrete time signals containing information is transmitted.

Energy storage may refer to an apparatus or means for electrochemical storage of energy such as batteries. The energy storage may comprise primary batteries or secondary, i.e. rechargeable, batteries. Some types of rechargeable batteries that may be used include lithium-ion (Li-ion) batteries, nickel cadmium (Ni—Cd) batteries, or Ni-metal hydride (Ni-MH) batteries. A single battery cell may be used. Alternatively, several battery cells may be coupled in parallel or series to form the energy storage. Energy storage may have a voltage in the range from 0.5V to 12V and feature an energy storage capacity in the range of 10 to 5000 mAh.

An energy receiver may refer to an apparatus or means for receiving energy at the implant from external devices or transmitters of energy. The receiver may be adapted to receiver energy conductively, via an electric conductor, in which case the energy being transmitted and received may be in an electrical form, e.g. a current or a voltage. The receiver may be adapted to receive energy wirelessly, in which case the energy may be in an electromagnetic wave form, e.g. a radio wave or a light pulse. The energy receiver may be adapted to directly operate a function of the implant or replenish an energy level of the energy storage.

A sensation generator is a device or unit that generates a sensation. The sensation generated may be configured to be experienceable by the patient such that the patient may take actions to authenticate a device, connection, or communication. The sensation generator may be configured to generate a single sensation or a plurality of sensation components. The sensation or sensation components may comprise a vibration (e.g. a fixed frequency mechanical vibration), a sound (e.g. a superposition of fixed frequency mechanical vibrations), a photonic signal (e.g. a non-visible light pulse such as an infra-red pulse), a light signal (e.g. a visual light pulse), an electric signal (e.g. an electrical current pulse) or a heat signal (e.g. a thermal pulse). The sensations generated by the sensation generator may be configured to be experienceable by a sensory function or a sense of the patient from the list of tactile, pressure, pain, heat, cold, taste, smell, sight, and hearing. Sensations may be generated of varying power or force as to adapt to sensory variations in the patient. Power or force may be increased gradually until the patient is able to experience the sensation. Variations in power or force may be controlled via feedback. Sensation strength or force may be configured to stay within safety margins. The sensation generator may be connected to the implant. The sensation generator may be comprised within the implant or be a separate unit.

A motor, e.g. of the active device or unit of the implant, for controlling a physical function in the body of the patient may provide a secondary function as a sensation generator, generating a vibration or sound. Generation of vibrations or sounds of the motor may be achieved by operating the motor at specific frequencies. When functioning as to generate a sensation the motor may operate outside of its normal ranges for frequency controlling a physical function in the body. The power or force of the motor when operating to generate a sensation may also vary from its normal ranges for controlling a physical function in the body.

An external device is a device which is external to the patient in which the implant is implanted in. The external device may be also be enumerated (first, second, third, etc.) to separate different external devices from each other. Two or more external devices may be connected by means of a wired or wireless communication as described above, for example through IP (internet protocol), or a local area network (LAN). The wired or wireless communication may take place using a standard network protocol such as any suitable IP protocol (IPv4, IPv6) or Wireless Local Area Network (IEEE 802.11), Bluetooth, NFC, RFID etc. The wired or wireless communication may take place using a proprietary network protocol. Any external device may also be in communication with the implant using wired or wireless communication according to the above. Communication with implanted devices may be thus accomplished with a wired connection or with wireless radiofrequency (RF) telemetry. Other methods of wireless communication may be used to communicate with implants, including optical and ultrasound. Alternatively, the concept of intrabody communication may be used for wireless communication, which uses the conductive properties of the body to transmit signals, i.e. conductive (capacitive or galvanic) communication with the implant. Means for conductive communication between an external device and an implant may also be called “electrical connection” between an external device and an implant. The conductive communication may be achieved by placing a conductive member of the external device in contact with the skin of the patient. By doing this, the external device and/or the implant may assure that it is in direct electrical connection with the other device. The concept relies on using the inherent conductive or electrical properties of a human body. Signals may preferably be configured to affect the body or body functions minimally. For conductive communication this may mean using low currents. A current may flow from an external device to an implant or vice versa. Also, for conductive communication, each device may have a transceiver portion for transmitting or receiving the current. These may comprise amplifiers for amplifying at least the received current. The current may contain or carry a signal which may carry e.g. an authentication input, implant operation instructions, or information pertaining to the operation of the implant.

Alternatively, conductive communication may be referred to as electrical or ohmic or resistive communication.

The conductive member may be an integrated part of the external device (e.g. in the surface of a smartwatch that is intended to be in contact with the wrist of the person wearing it), or it may be a separate device which can be connected to the external device using a conductive interrace such as the charging port or the headphone port of a smartphone.

A conductive member may be considered any device or structure set up for data communication with the implant via electric conductive body tissue. The data communication to the implant may be achieved by e.g. current pulses transmitted from the conductive member through the body of the patient to be received by a receiver at the implant. Any suitable coding scheme known in the art may be employed. The conductive member may comprise an energy source such as a battery or receive energy from e.g. a connected external device.

The term conductive interface is representing any suitable interface configured for data exchange between the conductive member and the external device. The conductive member may in an alternative configuration receive and transmit data to the external device through a radio interface, NFC, and the like.

An external device may act as a relay for communication between an implant and a remote device, such as e.g. second, third, or other external devices. Generally, the methods of relaying communication via an external device may be preferable for a large number of reasons. The transmission capabilities of the implant may be reduced, reducing its technical complexity, physical dimensions, and medical effects on the patient in which the implant is implanted. Communication may also be more efficient as direct communication, i.e. without a relaying device, with an implant from a remote device may require higher energy transmissions to account for different mediums and different rates of attenuation for different communication means. Remote communication with lower transmission energy may also increase the security of the communication as the spatial area or volume where the communication may be at all noticeable may be made smaller. Utilizing such a relay system further enables the use of different communication means for communication with the implant and communication with remote devices that are more optimized for their respective mediums.

An external device may be any device having processing power to perform the methods and functions needed to provide safe operation of the implant and provide the patient or other stakeholders (caregiver, spouse, employer etc.) with information and feedback from the implant. The external device may for example be a handset such as a smartphone, smartwatch, tablet etc. handled by the patient or other stakeholders. The external device may be a server or personal computer handled by the patient or other stakeholders. The external device may be cloud based or a virtual machine. In the drawings, the external device handled by the patient is often shown as a smart watch, or a device adapted to be worn by the patient at the wrist of the patient. This is merely by way of example and any other type of external device, depending on the context, is equally applicable.

Several external devices may exist such as a second external device, a third external device, or another external device. The above listed external devices may e.g. be available to and controllable by a patient, in which an implant is implanted, a caregiver of the patient, a healthcare professional of the patient, a trusted relative of the patient, an employer or professional superior of the patient, a supplier or producer of the implant or its related features. By controlling the external devices may provide options for e.g. controlling or safeguarding a function of the implant, monitoring the function of the implant, monitoring parameters of the patient, updating or amending software of the implant etc.

An external device under control by a supplier or producer of the implant may be connected to a database comprising data pertaining to control program updates and/or instructions. Such database may be regularly updated to provide new or improved functionality of the implant, or to mitigate for previously undetected flaws of the implant. When an update of a control program of an implant is scheduled, the updated control program may be transmitted from the database in a push mode and optionally routed via one or more further external devices before received by the implant. In another embodiment, the update is received from the database by request from e.g. an external device under control by the patient having the implant implanted in his/her body, a pull mode.

The external device may require authentication to be operated in communication with other external devices or the implant. Passwords, multi-factor authentication, biometric identification (fingerprint, iris scanner, facial recognition, etc.) or any other way of authentication may be employed.

The external device may have a user interface (UI) for receiving input and displaying information/feedback from/to a user. The UI may be a graphical UI (GUI), a voice command interface, speaker, vibrators, lamps, etc.

The communication between external devices, or between an external device and the implant may be encrypted. Any suitable type of encryption may be employed such as symmetric or asymmetric encryption. The encryption may be a single key encryption or a multi-key encryption. In multi-key encryption, several keys are required to decrypt encrypted data. The several keys may be called first key, second key, third key, etc. or first part of a key, second part of the key, third part of the key, etc. The several keys are then combined in any suitable way (depending on the encryption method and use case) to derive a combined key which may be used for decryption. In some cases, deriving a combined key is intended to mean that each key is used one by one to decrypt data, and that the decrypted data is achieved when using the final key.

In other cases, the combination of the several key result in one “master key” which will decrypt the data. In other words, it is a form of secret sharing, where a secret is divided into parts, giving each participant (external device(s), internal device) its own unique part. To reconstruct the original message (decrypt), a minimum number of parts (keys) is required. In a threshold scheme this number is less than the total number of parts (e.g. the key at the implant and the key from one of the two external device are needed to decrypt the data). In other embodiments, all keys are needed to reconstruct the original secret, to achieve the combined key which may decrypt the data.

In should be noted that it is not necessary that the generator of a key for decryption is the unit that in the end sends the key to another unit to be used at that unit. In some cases, the generator of a key is merely a facilitator of encryption/decryption, and the working in behalf of another device/user.

A verification unit may comprise any suitable means for verifying or authenticating the use (i.e. user authentication) of a unit comprising or connected to the verification unit, e.g. the external device. For example, a verification unit may comprise or be connected to an interface (UI, GUI) for receiving authentication input from a user. The verification unit may comprise a communication interface for receiving authentication data from a device (separate from the external device) connected to the device comprising the verification unit. Authentication input/data may comprise a code, a key, biometric data based on any suitable techniques such as fingerprint, a palm vein structure, image recognition, face recognition, iris recognition, a retinal scan, a hand geometry, and genome comparison, etc. The verification/authentication may be provided using third party applications, installed at or in connection with the verification unit.

The verification unit may be used as one part of a two-part authentication procedure. The other part may e.g. comprise conductive communication authentication, sensation authentication, or parameter authentication.

The verification unit may comprise a card reader for reading a smart card. A smart card is a secure microcontroller that is typically used for generating, storing, and operating on cryptographic keys. Smart card authentication provides users with smart card devices for the purpose of authentication. Users connect their smart card to the verification unit. Software on the verification unit interacts with the keys material and other secrets stored on the smart card to authenticate the user. In order for the smart card to operate, a user may need to unlock it with a user-PIN. Smart cards are considered a very strong form of authentication because cryptographic keys and other secrets stored on the card are very well protected both physically and logically, and are therefore hard to steal.

The verification unit may comprise a personal e-ID that is comparable to, for example, passport and driving license. The e-ID system comprises is a security software installed at the verification unit, and a e-ID which is downloaded from a web site of a trusted provided or provided via a smart card from the trusted provider.

The verification unit may comprise software for SMS-based two-factor authentication. Any other two-factor authentication systems may be used. Two-factor authentication requires two things to get authorized: something you know (your password, code, etc.) and something you have (an additional security code from your mobile device (e.g. a SMS, or a e-ID) or a physical token such as a smart card).

Other types of verification/user authentication may be employed. For example, a verification unit which communicate with an external device using visible light instead of wired communication or wireless communication using radio. A light source of the verification unit may transmit (e.g. by flashing in different patterns) secret keys or similar to the external device which uses the received data to verify the user, decrypt data or by any other means perform authentication. Light is easier to block and hide from an eavesdropping adversary than radio waves, which thus provides an advantage in this context. In similar embodiments, electromagnetic radiation is used instead of visible light for transmitting verification data to the external device.

In some embodiments, the data transmitted between the implant and an external device may be encrypted and/or decrypted with public and/or private keys. In some examples, the communication unit or the implant may comprise a private key and a corresponding public key, and the external device may comprise a private and a corresponding public key. The communication unit and the external device may exchange public keys and the communication may thus be performed using public key encryption. The person skilled in the art may utilize any known method for exchanging the keys. The communication unit may encrypt data to be sent to the external device using a public key corresponding to the external device. The encrypted data may be transmitted over a wired, wireless, or electrical/conductive communication channel to the external device. The external device may receive the encrypted data and decode it using the private key comprised in the external device, the private key corresponding to the public key with which the data has been encrypted. The external device may transmit encrypted data to the communication unit of the implant. The external device may encrypt the data to be sent using a public key corresponding to the private key of the implant. The external device may transmit the encrypted data over a wired, wireless, or electrical/conductive connection directly or indirectly, to the communication unit of the implant. The communication unit may receive the data and decode it using the private key comprised in the implant or in the communication unit.

In an alternative to the public key encryption, the data to be sent between an implant and an external device or between an external device and the implant may be signed. Data transmitted from the transmitting one of the implant and the external device may be signed using the private key of transmitting one. The receiving one of external device or the implant may receive the message and verify the authenticity of the data using the public key corresponding to the private key used for the signing. In this way, the receiving one of external device or the implant may determine that the sender of the data was correct and not from another device or source.

Parameters relating to fuinctionality of the implant may comprise for example a status indicator of the implant such as battery level, version of control program, properties of the implant, status of a motor of the implant, etc.

Data comprising operating instructions sent to the implant may comprise a new or updated control program, parameters relating to specific configurations of the implant, etc. Such data may for example comprise instructions how to operate the body engaging unit (active unit etc.) of the implant, switch body engaging unit in a multi functionality implant, instructions to collect patient data at the implant, instructions to transmit feedback from the implant to the external device, etc.

The expressions “confirming the electrical connection between an implant and an external device” or “authenticating a connection between an implant and an external device”, or similar expressions, are intended to encompass methods and processes for ensuring or be reasonably sure that the connection has not been compromised. Due to weaknesses in the wireless communication protocols, it is a simple task for a device to ‘listen’ to the data and grab sensitive information, e.g. personal data regarding the patient sent from the implant, or even to try to compromise (hack) the implant by sending malicious commands or data to the implant. Encryption may not always be enough as a security measure (encryption schemes may be predictable), and other means of confirming or authenticating the external device being connected to the implant may be needed.

The expression “network protocol” is intended to encompass communication protocols used in computer networks a communication protocol is a system of rules that allow two or more entities of a communications system to transmit information via any kind of variation of a physical quantity. The protocol defines the rules, syntax, semantics and synchronization of communication and possible error recovery methods. Protocols may be implemented by hardware, software, or a combination of both.

Communication protocols have to be agreed upon by the parties involved. In this field, the term “standard” and “proprietary” is well defined. A communication protocol may be developed into a protocol standard by getting the approval of a standards organization. To get the approval the paper draft needs to enter and successfully complete the standardization process. When this is done, the network protocol can be referred to a “standard network protocol” or a “standard communication protocol”.

Standard protocols are agreed and accepted by whole industry. Standard protocols are not vendor specific. Standard protocols are often, as mentioned above, developed by collaborative effort of experts from different organizations.

Proprietary network protocols, on the other hand, are usually developed by a single company for the devices (or Operating System) which they manufacture. A proprietary network protocol is a communications protocol owned by a single organization or individual. Specifications for proprietary protocols may or may not be published, and implementations are not freely distributed. Consequently, any device may not communicate with another device using a proprietary network protocol, without having the license to use the proprietary network protocol, and knowledge of the specifications for proprietary protocol. Ownership by a single organization thus gives the owner the ability to place restrictions on the use of the protocol and to change the protocol unilaterally.

A control program is intended to define any software used for controlling the implant. Such software may comprise an operating system of the implant, of parts of an operating system or an application running on the implant such as software controlling a specific functionality of the implant (e.g. the active unit of the implant, feedback functionality of the implant, a transceiver of the implant, encoding/decoding functionality of the implant, etc.). The control program may thus control the medical function of the implant, for example how much insulin the implant should deliver, etc. Alternatively, or additionally, the control program may control internal hardware functionality of the implant such as energy usage, transceiver functionality, etc.

The systems and methods disclosed hereinabove may be implemented as software, firmware, hardware, or a combination thereof. In a hardware implementation, the division of tasks between functional units referred to in the above description does not necessarily correspond to the division into physical units; to the contrary, one physical component may have multiple functionalities, and one task may be carried out by several physical components in cooperation. Certain components or all components may be implemented as software executed by a digital signal processor or microprocessor or be implemented as hardware or as an application-specific integrated circuit. Such software may be distributed on computer readable media, which may comprise computer storage media (or non-transitory media) and communication media (or transitory media). As is well known to a person skilled in the art, the term computer storage media includes both volatile and non-volatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information, and which can be accessed by a computer. Further, it is well known to the skilled person that communication media typically embodies computer readable instructions, data structures, program modules or other data in a modulated data signal such as a carrier wave or other transport mechanism and includes any information delivery media.

The system and methods disclosed herein will now be generally exemplified using patients with different implants as use cases. This is just by way of example to aid the understanding of the disclosure to the reader and should not be seen as limiting the scope of the disclosure in any way.

In the first use case, the system and methods are exemplified using patients with implanted insulin pumps. An implanted insulin pump is a pump device designed to be implanted in the body and deliver insulin, preferably into the peritoneal cavity, of patients suffering from diabetes. The peritoneal cavity holds a vast amount of blood vessels that are routed straight to the liver, which makes injection here a fast and effective way of delivering insulin. The insulin comprised in the pump is delivered through a catheter, and a medical professional will have to refill the pump through a refill port when the insulin has run out. The implanted insulin pump usually comes with a handheld controller that can be controlled by the patient or doctor and communicates wirelessly with the pump. With the controller, the patient can set basal patterns or deliver boluses. Some pumps are fully automated and delivers the right amount of insulin based on measured sugar levels, thus requiring little controller input from the user. Some pumps can comprise sensors that measure physiological parameters, such as temperature, pulse, glucose levels etc., The measured data can then be securely communicated, as described herein with reference to aspects 1-14, from the insulin pump to the patient or physician (healthcare provider) in order to adjust the delivery settings of the pump. The delivery setting can then be securely communicated from an external device to the insulin pump, as described herein with reference to aspects 1-14, The patient could for example provide authentication by speaking such that the implanted pump, provided with necessary means for sensing audio, registers the voice and thereby provides the measured data as for example numeral values displayed on the controller. By demanding such authorization, it is ensured that no unauthorized persons gain access to the patient's medical status or that the adjustment of the delivery settings is done by an unauthorized device.

Should the patient visit a hospital to get his/her pump refilled, a similar authorization process would take place between the implant and the doctor. The manufacturer of the pump can provide updates to the software in the pump in order to optimize its performance. Such updates must also be approved and authorized by the doctor and/or patient. This kind of authorization chain ensures that no unauthorized persons, such as passers-by or unauthorized personnel at a hospital, can gain access to the patient's medical data or alter the settings on the device.

In the second use case, the system and methods are exemplified using patients with implanted devices for treating obesity. The purpose of most implanted device for treating obesity is to control the patient's appetite. The implant, such as the one described in WO2009/096859 (the content of which is hereby incorporated by reference), can comprise a stretching device which is inserted in the stomach such that it is surrounded by a portion of the stomach wall. When enlarging the device, its circumference is increased, thereby stretching the surrounding stomach wall. Therefore, by expanding and/or constricting the stretching device, the receptors in the stomach can be manipulated to indicate the feeling of a full and/or empty stomach. This creates or impacts the feeling of satiety to the patient. The expansion and contraction of the stretching device is controlled by a control unit which can be controlled either automatically or directly by the patient. The patient could for example have a switch placed subcutaneously, which he or she can control from the outside of his or her body by applying pressure. When eating, the patient can press the switch, thereby controlling the size of the stretching device, and indirectly the feeling of satiety. The constriction and/or expansion of the stretching device can be performed by for example hydraulic, mechanical, or electrical means. Some implants for affecting the feeling of satiety can have stretching device that comprise multiple parts, mechanical or hydraulic. The different parts engage and stretch different parts of the stomach wall. In these implants, the different parts are adapted to be stretched independently from each other with regards to the force used for stretching the wall, as well as the time period during which the stretching occurs and the time at which the stretching occurs. The patient, or caregiver, could also wirelessly control the stretching of the device from outside the body by utilizing a wireless remote control, such as a mobile smart phone, that communicates with the implant. In some cases, the implant also comprises a sensor which senses physical parameters related to the patient, such as body temperature, blood pressure or blood flow. It is especially useful to measure parameters relating to the patient's food intake. The sensor could for example be adapted to sense an esophagus movement, muscle activity or stomach pressure. The control device, in communication with the sensor, can then control the stretching of the device based on the sensed parameters. In cases where the sensor is placed in the esophagus, parameters relating to for example the movement, bending, motility, stretching or pressure of the esophagus can be sensed. Placing a sensor in or in relation to the esophagus is especially advantageous since the movement pattern of the esophagus directly relates to the patient's food intake. Such sensor placed in the esophagus could for example be a strain gauge or any other sensor adapted to sense mechanical strain. When a patient having an implant as described above, or in WO2009/096859, wants to eat, he or she can activate the implant either by pressing the switch or, wirelessly through a mobile phone with an app connected to the implant. Depending on the size of the meal, number of calories, feeling of hunger or other parameter relating to the food intake, the patient can adapt the stretching by using different settings on the switch or app. The wireless communication between the implant and the app can be securely executed according to the aspects 1-14 described herein. The communication between the implant and the app does not always have to be encrypted, and technologies such as NFC can be utilized for less sensitive data. The patient could for example, prior to eating, provide information in the app that will control the stretching of the implant. Such information could for example be feeling of hunger, time since last meal or parameters relating to the food, such as estimated number of calories or food weight. The patient could also take a picture of the food, which the app can analyze. By analyzing the photo, the app can estimate parameters such as calories and then adjust the implant properly. In order to provide the optimal obesity treatment, the control of the implant, i.e. the app in this case, must be calibrated. This calibration could for example entail a text message being sent to the patient post eating, in which the patient is asked to rate his feeling of satiety. This text message could come automatically after each meal to continuously calibrate the implant. If the patient's answer does not correspond to the level of stretching of the implant, the stretching must be adjusted accordingly. Should the patient for example not experience a feeling of satiety after eating a big meal, the app must send instructions to the implant to increase the stretching. In the cases where multiple different stretching parts are utilized, the adjusting can sometimes be done by altering which part(s) is being used. This could be done completely automatically without requiring any input from the user. It is important not only to protect the data being sent between the app and implant, but also to ensure that the information from the app is communicated to the right implant. Otherwise, someone could accidentally send instructions to another patient's implant, thereby inadvertently controlling their device. The calibration of the implant would not work either if the app accidently receives information from the wrong implant. To ensure that this does not happen, the app and implant can be synched. This synchronization could for example involve a sensor in the esophagus which measures the esophagus movement patterns. The app and implant are synched only if the measured movement pattern of the esophagus corresponds to the entered food data in the app. Another way to synchronize the implant with the app is to incorporate a gyroscope in the implant. The implant's gyroscope can then be controlled against the gyroscope in the patient's mobile phone. This of course requires that the patient has the mobile phone on him. Both of these synchronization methods ensure that no passer-by with an implant, accidentally or intentionally receives or sends instructions or information to another person's implant. The app on the phone could also have security measures in order to ensure that only authorized users control the app and implant. This authorization can be performed by for example voice or face recognition, allowing only the right user to enter data in the app. In cases where the implant comprises means for detecting sound, voice recognition could also be utilized for synchronizing the mobile phone with the implant.

After the synchronization, and after the information from the implant sensor (i.e. the esophagus movement pattern, abdominal movement or any other sensed physical parameter) has been communicated to the app, this information can be sent to a doctor and/or manufacturer for evaluation. This information is encrypted and communicated securely as described herein in aspects 1-14. This information can comprise not only the sensed implant parameters, but also added app information input by the patient. Such information could be anything from pictures of food he has eaten, weight or satiety status. All information can be utilized by the doctor and/or manufacturer to improve and calibrate the implant's behavior. Should the doctor or manufacturer want to update the settings or software of the implant, this is communicated securely, by means of aspects 1-14 as described herein, back to the app on the patient's mobile phone. Should the patient for example experience a sense of satiety despite having a low food intake, he or she enters this in the app and/or responds to the text message. This information is then communicated wirelessly to the doctor or medical professional, who in turn can evaluate this information and decide whether or not the implant must be adjusted. It is also possible to have a feature in the app in which a picture informs the user how much he or she should eat to feel satiety. The picture could instruct the user by showing which volume, weight and/or calorie amount the food should have in order to be adequate. How much food, measured in calories or volume, a patient should intake can be decided by the doctor, who then sends settings and/or instructions securely to the patient's app and/or device. The doctor and/or manufacturer could update the software on the app and send these updates directly to the app without requiring any action from the patient. The patient can be informed of the update by an email, text message, app notification or any other notification method. Anytime a doctor or employee at the manufacturing company wants to update or alter the settings of the implant he or she must be verified. This could for example be done by requiring electronic identification. To further ensure that the information being sent between the implant, the app, and the doctor and/or manufacturer's database is protected, blockchains can be utilized as defined below. It is also possible to allow another person, such as a family member, friend, or physician, to gain access to implant information or adjust the settings of the implant through the app on their mobile phone. This could for example be convenient if the patient himself is ill or in any other acute situations where the patient is not suited to manage the app himself. The authentication could then entail a verification code provided by the patient's app which the other user is required to enter on his app.

In the third use case, the system and methods are exemplified using patients with implanted devices for urinary control. Involuntary urinary retention is a condition in which the patient cannot empty the bladder completely. Besides the possibility of the condition being very painful if acute, it is also, among other, associated with urinary infections and renal damages. Patients suffering from this condition can benefit from an implant, such as the one described in WO2009048373, the content of which is hereby incorporated by reference. Such implant has a powered member which exerts a force on the urinary bladder which aids in the discharge of urine. The powered member can be controlled by a control device, such as a switch implanted subcutaneously at a, for the patient, convenient and easy to access location. When engaging the switch, a force is applied to the powered member which acts as a bladder press and presses against the outside of the urinary bladder and thereby releases urine. The implant can also comprise an artificial urinary sphincter, which acts as a urine stopper and is also controlled by the control device. When the control device activates the urine stopper, the artificial sphincter retracts which stops urine from exiting the urine bladder and entering the urethra. If the control device is a wireless device, such as a mobile phone, the implant, i.e. the bladder press and urine stopper, communicates securely and wirelessly with the mobile phone according to the aspects 1-14 described herein. The implant could also comprise sensors for sensing different physical parameters such as, pressure. This is especially essential since many patients with urinary dysfunctions cannot feel when the bladder needs to be emptied. The pressure sensor(s) can then indicate that the bladder is full to the control device. The patient could for example receive a text message or a notification in an app connected to the implant telling him that the bladder is full. The patient can then control the bladder pressure via his app at a convenient time as a regular toilet visit. It is also possible that the patient has an ultrasound sensor implanted for measuring the level of urine in the bladder. If the sensor indicates that there is little to no urine in the bladder, it can communicate this to the patient via the app, thereby letting him or her know that no toilet visit is necessary. The ultrasound sensor could also measure other features, such as the quality of the content in the bladder. Should the ultrasound sensor for example sense blood in the bladder, the patient can get notified, as this can be a sign of infection or kidney disease. Since the bladder pressure is attached to a support structure in the body, such as the pelvic bone, for exerting the force of the bladder pressure against the structure, the implant could also comprise sensors for sensing the mechanical strain. Should the patient experience that the bladder isn't fully emptied despite activation via the app, he or she can indicate this in the app which communicates this and adjusts the strain by tightening the attachment to the support structure. It is also possible that the sensors, without input from the patient, senses that the bladder is full (i.e. is expanded thereby influencing the strain) and alerts the patient that he or she needs to empty the bladder. The urine stopper, i.e. the artificial urinary sphincter, can be controlled in a similar manner. If the sensor(s) connected to the urine stopper senses that the patient is lying down (gyroscope sensor) it can release the pressure exerted on the urethra. The patient can indicate in the app that he or she is ready to sleep, thereby release the pressure over night. In the morning, he or she can increase the pressure again by informing the app that they intend to get up and start the day. After the patient has emptied his or her bladder, the pressure sensed by the sphincter's sensors will automatically go down. Should the patient however engage in physical activities, he or she can actively increase the pressure of the stopper by using the settings in the app. The patient can calibrate the implants by giving feedback in the app. If a certain bladder pressure setting does not fully empty the bladder, the strain in the attachment portion might have to be adjusted. Or if the patient indicates on a rating scale that he will engage in very heavy physical activity, and the urine stopper doesn't retract enough to keep the urine from leaking into the urethra, the patient can indicate this in the app. This will require the stopper to exert more force next time the patient engages in an equally rated activity. The muscles of the urinary bladder can be stimulated to contract the bladder and thereby prevent unwanted leakage. If the implant is further equipped with a stimulating device for electrically stimulating the muscles, the patient can indicate that he wishes to stimulate the muscles occasionally.

In the fourth use case, the system and methods are exemplified using patients with implanted devices for intestinal disorders. In a similar manner as the bladder presser, patients suffering from intestinal disorders can benefit from an implant that can empty a reservoir on demand. Patients suffering from intestinal disorders can have trouble to control the flow of intestinal contents, and especially to control when feces are exiting the patient's body. An implant as described in WO2011128124, the content of which is hereby incorporated by reference, is suitable for treating such patients. The implant acts on a reservoir formed from surgically modified intestine and comprises an implantable artificial flow control device. The flow control device can be a pump which reduces the reservoir's volume, thereby emptying it. Patient's with this type of implant can control the pump through an app on their mobile phone. Such wireless communication between the pump and app is securely performed by means of aspects 1-14 as described herein. The implant can further comprise sensors that can indicate parameters such as reservoir volume or pressure. When the sensors indicate that the reservoir is full, the implant communicates this to the patient's mobile phone via some sort of notification. The patient can then activate the pump as soon as convenient.

In the fifth use case, the system and methods are exemplified using patients with implanted devices for treating aneurysms. An aneurysm is a localized blood-filled dilation of a blood vessel. They most commonly occur in the arteries at the base of the brain, called Circle of Willis, and in the aorta. Aneurysms grow larger with time, therefore they exercise a great threat if left untreated. An implant for treating aneurysms is described in WO2008000574 and WO2009048378, the content of which is hereby incorporated by reference. These implants provide a member placed around the vessel on which a force can be applied. The implants can communicate wirelessly with an external unit, such as a mobile phone. Sensors can be present in the implant for sensing parameters relating to pressure, blood flow, and strain, among other things. Should the sensors detect an increase in pressure, if the aneurysm is about to burst, or if the patient is exercising, it notifies the mobile phone and thereby the patient. The patient could also prior to engaging in physical activities, actively increase the pressure the implant exerts on the vessel to prevent it from bursting. Should the sensors notice a too quick expansion, which might indicate an acute burst, the implant could directly alert a doctor or medical emergency team. It could also trigger an alarm in the patient's mobile phone to alert the patient. It is possible to continuously measure parameters such as blood pressure, and send this information to an app on the patient's mobile phone. In this way, the patient can get continuous information on his or her aneurysm status.

In the sixth use case, the system and methods are exemplified using patients with implanted devices for treating heart arrhythmia. Heart arrhythmia are conditions relating to the electrical conduction system of the heart. In a healthy heart, the sinoatrial node located in the right atrium wall spontaneously sends electrical impulses causing the heart to contract regularly. In patient's suffering from arrhythmia however, a damaged sinoatrial node, or a blockage in the electrical pathways of the heart, causes too fast, too slow, or irregular heart contractions. An artificial pacemaker is an implantable medical device designed to monitor the heart and alleviate such conditions. The pacemaker is implanted just below the collarbone and provides electrical impulses to the heart through electrodes inserted through a large vein leading directly to the heart. Depending on the patient's specific condition, the artificial pacemaker may have electrodes placed in both the ventricle and atrium walls. The pacemaker continuously monitors the heart, and in many cases, it is programmed to only electrically stimulate the heart when the natural heart rate falls below a set lower limit. Since modern pacemakers are able to communicate wirelessly, remote physician follow-ups and remote continuous monitoring of the patient's heart is enabled, resulting in less travels back and forth to the hospital. If the physician recognizes that the pacemaker is malfunctioning somehow, the software can be remotely updated or altered. This does however make the pacemakers vulnerable to unauthorized people also gaining access to its data and/or altering its settings such as reducing battery life or increasing impulse activity. Therefore, it is important that the monitored data from the pacemaker is securely communicated as described herein with reference to aspects 1-14 to the physician. The data could for example be transferred to a secure server to which a physician can only gain access by identifying himself via an electronic identification card. Should he then want to alter the settings of the pacemaker, an update is sent back to the implant, likewise securely communicated as described herein with reference to aspects 1-14.

If a patient with an implanted pacemaker experiences any abnormal behavior related to the heart, such as chest pain, sudden drop/increase of heart rate or rapid or irregular pulse, he could authenticate himself to the pacemaker by any means describe herein with reference to aspects 1-14. An example of this could be utilizing face recognition on a smart phone. The physician in charge could then be contacted and gain access to the pacemakers monitored activity by for example authenticating himself using electronic identification as described above. Should the settings on the pacemaker need to be altered or the software updated, the physician can communicate this update and the authentication process might have to be repeated at both the physician's and patient's end. Some less sensitive data, such as reading the battery status of the implant, does not always have to be encrypted, and could be done by the patient at home by using methods such as NFC.

Of course, the reasoning above equally applies for patients with implantable cardio-defibrillators.

More summarily, the implant may e.g. comprise, be, or act as at least one of a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA,
an implant replacing an organ of the patient or part of an organ of the patient or the function thereof,
a vascular treatment device, and
an implant adapted to move fluid inside the body of the patient.

The implant may comprise an internal control unit adapted to be involved in at least a part of the actions performed by the implant.

All embodiments or part of embodiments in the different aspects herein could be combined with any and/or all other embodiments or parts thereof in any order, thus comprising; embodiments or parts thereof in the same aspect in any order or combined with any and/or all embodiments or parts thereof in any different aspect in any order. The connection herein between the aspects and any of its embodiments or parts thereof are just examples and they are intended to be combined with each other in any combination or order. The embodiments or parts thereof may therefore be connected to each other in any order of connection between the embodiments or parts thereof.

Thus, all the different aspects or parts thereof could be combined with each other in any combination. Any and/or all embodiments or parts thereof in one aspect could be combined with any and/or all embodiments or parts thereof in any and/or all other aspects, embodiment(s) or parts thereof, described elsewhere in any order or combination. For example, any of the embodiments labeled A-U may be combined with any of the numbered embodiments 244-258, 307-318, 330-331, 432-439, 447 and 453-459.

### Aspect 244SE Implantable Reset Switch—Implant Comprising a Reset Function—Embodiments of the Aspect 244SE of the Disclosure

In aspect 244SE, an implantable reset function and methods for controlling such implantable reset functions are provided. FIGS. 1-7 show embodiments of this aspect. Generally, the embodiments of the aspect 244SE provide a secure way of controlling the implant, updating the control program of the implant, and managing the control program of the implant.

A first embodiment of the aspect 244SE will now be described with reference to FIGS. 1a-c. FIG. 1a shows a patient with an implant 100. The implant 100 is in FIG. 1 placed in the abdominal area of the patient but could equally be placed in other parts of the body. The implant 100 comprises an active unit (further described in other sections of the description) 101 which is directly or indirectly connected to the body of the patient for performing a function in relation to the body of the patient. This function could for example be expanding and/or retracting a blood vessel, contracting muscles such as the heart, or in any other way perform a physical influence on the body. The active unit is connected to a communication unit 102 via an electrical connection C2. The communication unit 102 (further described with reference to FIG. 1B) is configured to communicate with an external device 200 (further described with reference to FIG. 1c). The communication unit 102 can communicate wirelessly with the external device 200 through a wireless connection W1, and/or through an electrical connection C1.

Referring now to FIG. 1B, the communication unit 102 will be describe in more detail. The communication unit 102 comprises an internal computing unit 106 configured to control the function performed by the implant 100. The computing unit 106 comprises an internal memory 107 configured to store programs thereon. The internal memory 107 comprises a first control program 110 which can control the function of the implant. The first control program may be seen as a program with minimum functionality to be run at the implant only during updating of the second control program. When the implant is running with the first control program, the implant may be seen as running in safe mode, with reduced functionality. For example, the first control program may result in that no sensor data is stored in the implant while being run, or that no feedback is transmitted from the implant while the first control program is running. By having a low complexity first control program, memory at the implant is saved, and the risk of failure of the implant during updating of the second control program is reduced.

The second control program is the program controlling the implant in normal circumstances, providing the implant with full functionality and features.

The memory 107 can further comprise a second, updatable, control program 112. The term updatable is to be interpreted as the program being configured to receive incremental or iterative updates to its code or be replaced by a new version of the code. Updates may provide new and/or improved functionality to the implant as well as fixing previous deficiencies in the code. The computing unit 106 can receive updates to the second control program 112 via the communication unit 102. The updates can be received wirelessly W1 or via the electrical connection C1. As shown in FIG. 1B, the internal memory 107 of the implant 100 can possibly store a third program 114. The third program 114 can control the function of the implant 100 and the computing unit 106 updates the second program 112 to the third program 114. The third program 114 can be utilized when rebooting an original state of the second program 112. The third program 114 may thus be seen as providing a factory reset of the implant 100, e.g. restore it back to factory settings. The third program 114 may thus be included in the implant in a secure part of the memory 107 to be used for resetting the software (second control program 112) found in the implant 100 to original manufacturer settings.

A reset function 116 is connected to or part of the internal computing unit 106 or transmitted to said internal computing unit. The reset function is configured to make the internal computing unit 106 switch from running the second control program 112 to the first control program 110. The reset function could be configured to make the internal computing unit 106 delete the second control program 112 from the memory 107. The reset function 116 can be operated by palpating or pushing/put pressure on the skin of the patient. This could be performed by having a button on the implant. Temperature sensors and/or pressure sensors can be utilized for sensing the palpating. The reset function 116 could also be operated by penetrating the skin of the patient. It is further plausible that the reset function 116 can be operated by magnetic means. This could be performed by utilizing a magnetic sensor and applying a magnetic force from outside the body. The reset function 116 could be configured such that it only responds to magnetic forces applied for a duration of time exceeding a limit, such as 2 seconds. The time limit could equally plausible be 5 or 10 seconds, or longer. In these cases, the implant could comprise a timer. The reset function 116 may thus include or be connected to a sensor for sensing such magnetic force.

The communication unit 102 can further comprise an internal wireless transceiver 108. The transceiver 108 communicates wirelessly with the external device 200 through the wireless connection W1. The communication unit 102 can further be electrically connected C1 to the external device 200 and communicate by using the patient's body as a conductor.

The confirmation/authentication of the electrical connection can be performed as described herein under the fifth, thirteenth or fifteenth aspect. In these cases, the implant and/or external device(s) comprises the necessary features and functionality (described in the respective sections of this document) for performing such confirmation/authentication. By authenticating according to these aspects, security of the authentication may be increased as it may require a malicious third party to know or gain access to either the transient physiological parameter of the patient or detect randomized sensations generated at or within the patient.

In FIGS. 1a-c the patient is a human, but other mammals are equally plausible. It is also plausible that the communication is performed by inductive means. It is also plausible that the communication is direct.

The communication unit 102 of the implant 100 according to FIG. 1B further comprises a feedback unit 149. The feedback unit 149 provides feedback related to the switching from the second control program 112 to the first control program 110. The feedback could for example represent the information on when the update of the software, i.e. the second control program 112, has started, and when the update has finished. This feedback can be visually communicated to the patient, via for example a display on the external device 200. This display could be located on a watch, or a phone, or any other external device 200 coupled to the communication unit 102. Preferably, the feedback unit 149 provides this feedback signal wirelessly W1 to the external device 200. Potentially, the words “Update started”, or “Update finished”, could be displayed to the patient, or similar terms with the same meaning. Another option could be to display different colors, where green for example could mean that the update has finished, and red or yellow that the update is ongoing. Obviously, any color is equally plausible, and the user could choose these depending on personal preference. Another possibility would be to flash a light on the external device 200. In this case the external device 200 comprises the light emitting device(s) needed. Such light could for example be a LED. Different colors could, again, represent the status of the program update. One way of representing that the update is ongoing and not yet finished could be to flash the light, i.e. turning the light on and off Once the light stops flashing, the patient would be aware of that the update is finished. The feedback could also be audible, and provided by the implant 100 directly, or by the external device 200. In such cases, the implant 100 and external device comprises means for providing audio. The feedback could also be tactile. In such case, either the implant 100 or external device comprises means for providing a tactile sensation, such as a vibration.

As seen in FIG. 1B, the communication unit 102 can further comprise a first power supply 10a. The first power supply 10a runs the first control program 110. The communication unit 102 further comprises a second power supply 10b which runs the second control program 112. This may further increase security during update, since the first control program has its own separate energy supply. The first power supply 10a can comprise a first energy storage 104a and/or a first energy receiver 105a. The second power supply 10b can comprise a second energy storage 104b and/or a second energy receiver 105b. The energy can be received wirelessly by inductive or conductive means. An external energy source can for example transfer an amount of wireless energy to the energy receiver 105a, 105b inside the patient's body by utilizing an external coil which induces a voltage in an internal coil (not shown in figures). It is plausible that the first energy receiver 105a receives energy via a RFID pulse. The feedback unit 149 can the provide feedback pertaining to the amount of energy received via the RFID pulse. The amount of RFID pulse energy that is being received can be adjusted based on the feedback, such that the pulse frequency is successively raised until a satisfying level is reached.

The external device is represented in FIG. 1c. In the first embodiment, the external device 200 is placed around the patient's arm. It is equally plausible that the external device is placed anywhere on the patient's body, preferably on a convenient and comfortable place. The external device 200 could be a wristband, and/or have the shape of a watch. It is also plausible that the external device is a mobile phone or other device not attached directly to the patient. The external device as shown in FIG. 1c comprises a wired transceiver 203, and an energy storage 204. It also comprises a wireless transceiver 208 and an energy transmitter 205. It further comprises a computing unit 206 and a memory 207. The feedback unit in the external device 200 is configured to provide feedback related to the computing unit 206. The feedback provided by the feedback unit 210 could be visual. The external device 200 could have a display showing such visual feedback to the patient. It is equally plausible that the feedback is audible, and that the external device 200 comprises means for providing audio. The feedback given by the feedback unit 210 could also be tactile, such as vibrating. The feedback could also be provided in the form of a wireless signal W1.

FIG. 2A shows another embodiment of the aspect 244SE in which a patient has an implant 100 and an external device 200 in the form of a mobile phone. The external device 200 communicates wirelessly W1 with the internal communication unit 102. The external device 200 is shown in FIG. 2B.

In FIG. 2A, the external device 200 is displayed as a mobile phone, however, it is equally plausible that the external device 200 is a watch, necklace, or any other wearable unit. Preferably, the external device is at least one of small, portable, easy to access, inconspicuous and/or easy to disguise as part of a patient's daily look.

FIG. 2B shows the external device 200 in the form of a mobile phone. The external device 200 comprises all of the features as described earlier with reference to FIG. 1c.

Such an implant 100 as described in this aspect 244SE, with, or alternatively in electrical or wireless connection with, the reset function 116 further increases the security of the communication with and the operation of the implant 100 as the reset function 116 may be under the direct control of the patient in which the implant is implanted. In a case where the reset function is implanted or comprised within an implanted implant 100, extra security is granted as an effect of the reset function location being non-obvious to a malicious third party aiming to access or affect the implant 100.

A method for controlling an implantable reset function according to the aspect 244SE will now be described with reference to FIGS. 3-7. It is to be understood that the implant referred to in FIGS. 3-7 may comprise all required features described earlier with reference to FIGS. 1-2.

FIG. 3 shows a method for controlling the function of an implant 100. The reset function 116 is activated S4401, and then the internal computing unit 106 is instructed S4402 by the reset function 116 to switch from running the second control program 112 to running the first control program 110. Optionally, the internal computing unit 106 deletes S4403 the second control program 112 from the internal memory 107. For some embodiments, the memory 107 is configured to store a third control program 114. The method then includes updating S4404 the second program 112 to the third program 114. The third program 114 can for example be utilized when rebooting to an original state of the second program 112. As can be seen in FIG. 3, the internal computing unit 106 can then switch S4405 from running the first program 110 to running the updated second program 112. FIG. 4 shows a method for controlling an implant 100 in which after the activation S4401 of the reset function 116, and after switching S4402 from running the second program 112 to the first program 110, an update of the second program 112 is communicated S4414 from the external device 200 to the internal communication unit 102. This is followed by switching S4405 from running the first program 110 to running the update second program 112. FIG. 5 shows various ways of activating S4401 the reset function 116. The function 116 can for example be activated by palpating S4401a the skin of the patient. Palpating is to be understood as applying pressure to the skin, by means of for example the patient's or a doctor's hand(s). The activation S4401 could also be performed by penetrating S4401b the skin of the patient. This could for example entail penetrating S4401b the skin of the patient using a needle or other suitable medical equipment. The reset function 116 may thus comprise a push button or similar with a suitable form/function to be activated by the penetration S4401b of the skin using the applicable equipment. It is equally plausible that the activation S4401 is performed by applying a magnetic force S4401c from outside the body of the patient. In such case, the implant 100 and/or external device 200 comprises means for applying and/or sensing such magnetic force. After the activation S4401 is done, the computing unit 106 switches S4402 from running the second program 112 to running the first program 110. FIG. 6 shows a method for controlling the implant 100 by activating S4401 the reset function 116, switching S4402 from running the second program 112 to running the first program 110, and then providing S4423 feedback related to the switching of programs. This feedback is performed by means of a feedback unit 149 as described in detail with reference to FIGS. 1a-c. FIG. 7 shows a method for controlling an implant 100 in which after the switching S4402 is performed, energy is provided S4433 to the first energy receiver 105a by an energy transmitter 205 of the external device 200. The energy could for example be provided using RFID pulses. In the method of FIG. 7, the feedback unit 149 provides feedback to the energy transmitter 205 pertaining to the amount of RFID energy received. A parameter such as the frequency and/or amplitude of a subsequent RFID pulse can then be adjusted S4435 based on the feedback. It is further possible to have a computer program product with a computer-readable storage medium with instructions, that can carry out the methods as described herein with reference to FIGS. 3-7 when executed by a device with processing capability (not shown). The communication referred to with respect to the embodiment of the aspect 244SE and the accompanying FIGS. 1-7 can be securely performed as described herein under the second, third, sixth, seventh and tenth aspects. In these cases, the implant and/or external device(s) comprises the necessary features and functionality (described in the respective sections of this document) for performing such secure communication. For example, the data communicated from the external device 200 to the implant 100 comprising an update of the second control program may advantageously be encrypted, for example as described herein under the second or third aspect.

The reset function 116 may be a reset switch. Such a reset switch 116 may be in the form of an electrical switch. The reset switch 116 may alternatively be in the form of a magnetic switch. The reset switch 116 may be based on application of mechanical pressure. The reset switch 116 may be spring loaded to automatically flip back once pressure is no longer applied.

The internal computing unit 106 may be configured for receiving, from said external device 200, an update of the second control program 112,
updating the second control program 112,
switching, by the internal computing unit 106, from running said first control program 110 to running said second program 112 after updating the second control program 112.

The reset functionality of the implant 100, as discussed in the above, may be utilized in the case that the main control program, e.g. the second control program 112, malfunctions. An example of a malfunction could be if the active control program fails to control mechanical actuators or an active device 101 of the implant 100. The failing mechanical actuator could for example fail in performing its objective of opening and closing a noose around a urinary tract of a patient.

In such cases, the reset functionality may be utilized to reset and/or amend the control program with the aim of resolving the issues by fixing the control program or at least restoring it to a more stable or uncorrupted version.

The functionality may further be utilized as the control program is regularly updated, without any underlying malfunction forcing swift action to be taken.

The reset function 116 may be triggered by an update of the first or second control program 110, 112.

The reset function 116 may be triggered by a malfunction of the first or second control program 110, 112.

The reset function 116 may be triggered by a malfunction of an active device 101 of the implant 100.

The reset function 116 may be configured to be operated by Near Field Communication (NFC).

The reset function 116 may be configured to trigger implant diagnostics to be transmitted from the implant 100 to the external device 200. The implant diagnostics may comprise information or data pertaining to an error mode, an error code, or other diagnostics of the implant 100.

The reset function 116 may be configured to be operated by said magnetic force being applied at least two times. A reset may be triggered after e.g. two magnetic forces applications have been detected within a set time interval. The implant 100 may comprise at least one Hall element for detecting externally applied magnetic forces.

The first energy receiver 105a may be configured to receive energy conductively or inductively. As such, the need for an emergency battery may be negated.

The reset function 116 may be configured to be triggered if the first energy receiver 105a is receiving energy.

The first control program 110 may be configured to be running, powered by conductively or inductively received energy. This may be advantageous for emergency powering.

Said amount of energy received via the RFID pulse may be encoded in a variable pulse feedback signal provided by the feedback unit 149. The amount of energy received may be encoded in a frequency, an amplitude, an offset, a duty cycle, or a waveform of the variable pulse feedback signal.

The implant may comprise at least one of:
a pacemaker unit or implantable cardioverter defibrillators,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant controlling the emptying of a urinary bladder,
an implant hindering urinary leakage,
an implant hindering anal incontinence,
an implant controlling the emptying of fecal matter,
an implant monitoring an aneurysm,
an implant lubricating a joint,
an implant with a reservoir for holding bodily fluids
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling a drug treatment parameter,
an implant controlling a parameter in the blood,
an active electrically controlled implant devoid of an electrical heart stimulation system,
an active electrically controlled non-heart stimulation implant,
an implant adapted for electrical stimulation of muscles, a non-nerve stimulation system,
an active non-stimulation implant,
an implant for high current electrical stimulation defined as current above 1 mA or current above 5 mA, 10 mA, or 20 mA,
an implant replacing an organ of the patient or part of an organ of the patient or the function thereof,
a vascular treatment device, and
an implant adapted to move fluid inside the body of the patient.

The implant may comprise an internal control unit adapted to be involved in at least a part of the actions performed by the implant.

### Aspect 245SE 2-Part Key—Encrypted Communication Between Implant and External Device—Embodiments of Aspect 245SE of the Disclosure

In aspect 245SE, increased security for communication between an external device(s) and an implant is provided. FIGS. 8-17 shows embodiments of this aspect.

A first embodiment of aspect 245SE will now be described in conjunction with FIGS. 8A-C and 12. In this embodiment, a method of communication between an external device 200 and an implant 100 is provided, when the implant 100 is implanted in a patient and the external device 200 is positioned external to the body of the patient. The external device 200 is adapted to be in electrical connection C1 with the implant 100, using the body as a conductor. The electrical connection C1 is used for conductive communication between the external device 200 and the implant 100. The implant 100 comprises a communication unit 102. Both the implant 100 and the external device 200 comprises a wireless transceiver 108, 208 for wireless communication C1 between the implant 100 and the external device 200. The wireless transceiver 108 (included in the communication unit 102) may in some embodiments comprise sub-transceivers 1091, 1092 for receiving data from the external device 200 and other external devices, e.g. using different frequency bands, modulation schemes etc.

In a first step of the method of FIG. 12, the electrical connection C1 between the implant 100 and the external device 200 is confirmed S4501 and thus authenticated. The confirmation and authentication of the electrical connection may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

The implant may comprise a first transceiver 103 configured to be in electrical connection C1 with the external device, using the body as a conductor. The external device may comprise a first external transmitter 203 configured to be in electrical connection C1 with the implant, using the body as a conductor, and the wireless transmitter 208 configured to transmit wireless communication W1 to the implant 100. The first transmitter 203 of the external device may be wired or wireless. The first transmitter 203 and the wireless transmitter 208 may be the same or separate transmitters. The first transceiver 103 of the implant 100 may be wired or wireless. The first transceiver 103 and the wireless transceiver 102 may be the same or separate transceivers. The implant 100 may comprise a computing unit 106 configured to confirm the electrical connection between the external device 200 and the internal transceiver 103 and accept wireless communication W1 (of the data) from the external device 200 on the basis of the confirmation.

Data is transmitted S4502 from the external device 200 to the implant 100 wirelessly, e.g. using the respective wireless transceiver 108, 208 of the implant and the external device. Data may alternatively be transmitted through the electrical connection. As a result of the confirmation, the received data is used S4503 for instructing the implant. For example, as shown in FIG. 17, a control program 110 running in the implant 100 may be updated S4541, the implant 100 may be operated S4542 using operation instructions in the received data. This may be handled by the computing unit 106.

The embodiment of FIG. 12 may be extended to further increase security. This will be described below. The step S4502 of transmitting data from the external device 200 to the implant 100 wirelessly comprises transmitting S4512 encrypted data wirelessly. To decrypt the encrypted data (for example using the computing unit 106), several methods may be used.

In one embodiment, shown in FIG. 13, a key is transmitted using the confirmed conductive communication channel C1 (i.e. the electrical connection) from the external device 200 to the implant 100. The key is received S4505 at the implant (by the first internal transceiver 103). The key is then used for decrypting S4508 the encrypted data.

In some embodiments the key is enough to decrypt S4508 the encrypted data. In other embodiments, further keys are necessary to decrypt the data. In FIG. 14, one such embodiment is shown. In this embodiment, a key is transmitted using the confirmed conductive communication channel C1 (i.e. the electrical connection) from the external device 200 to the implant 100. The key is received S4505 at the implant (by the first internal transceiver 103). A second key is transmitted S4504 (by the wireless transceiver 208) from the external device 200 using the wireless communication W1 and received S4515 at the implant 100 by the wireless transceiver 108. The computing unit 106 is then deriving S4516 a combined key from the key and second key and uses this for decrypting S4518 the encrypted data.

In yet other embodiments, shown in FIG. 15 in conjunction with FIG. 10, a key is transmitted using the confirmed conductive communication channel C1 (i.e. the electrical connection) from the external device 200 to the implant 100. The key is received S4505 at the implant (by the first internal transceiver 103). A third key is transmitted S4524 from a second external device 300, separate from the external device 200, to the implant wirelessly W2. The third key may be received S4525 by a second wireless receiver 1092 (part of the wireless transceiver 108) of the implant 100 configured for receiving wireless communication W2 from second external device 300.

The first and third key may be used to derive S4526 a combined key by the computing unit 106, which then decrypts S4512 the encrypted data. The decrypted data is then used for instructing S4503 the implant 100 as described above.

The second external device 300 may be controlled by for example a caregiver, to further increase security and validity of data sent and decrypted by the implant 100.

It should be noted that in some embodiments shown in FIG. 11, the external device is further configured to receive W3 secondary wireless communication from the second external device 300, and transmit data received from the secondary wireless communication W3 to the implant. This routing of data may be achieved using the wireless transceivers 108, 208 (i.e. the wireless connection W1, or by using a further wireless connection W4 between the implant 100 and the external device 200. The routing may be performed as described herein under aspect 253SE. In these cases, the implant and/or external device(s) comprises the necessary features and functionality (described in the respective sections of this document) for performing such routing. Consequently, in some embodiments, the third key is generated by the second external device 300 and transmitted W3 to the external device 200 which routes the third key to the implant 100 to be used for decryption of the encrypted data. In other words, the step of transmitting a third key from a second external device, separate from the external device, to the implant wirelessly, comprises routing the third key through the external device. Using the external device 200 as a relay, with or without verification from the patient, may provide an extra layer of security as the external device 200 may not need to store or otherwise handle decrypted information. As such, the external device 200 may be lost without losing decrypted information.

In yet other embodiments, shown in FIG. 16 in conjunction with FIG. 10, a key is transmitted using the confirmed conductive communication channel C1 (i.e. the electrical connection) from the external device 200 to the implant 100. The key is received S4505 at the implant (by the first internal transceiver 103). A second key is transmitted S4514 from the external device 200 to the implant 100 wirelessly W1, received S4515 at the at the implant. A third key is transmitted S4524 from the second external device (300), separate from the external device, to the implant 100 wirelessly W2, received S4525 at the implant. Encrypted data transmitted S4512 from the external device 200 to the implant 100 is then decrypted S4538 using a derived S4526 combined key from the key, the second key and the third key.
wherein the external device is a wearable external device.

The external device 200 may be a handset.

The second external device 300 may be a handset.

The second external device 300 may be a server.

The second external device 300 may be cloud based.

In some embodiments, shown in FIG. 10, the electrical connection C1 between the external device 200 and the implant 100 is achieved by placing a conductive member 201, configured to be in connection with the external device 200, in electrical connection with a skin of the patient for conductive communication C1 with the implant. This feature may be achieved as described herein under aspect 247SE. In these cases, the implant and/or external device(s) comprises the necessary features and functionality (described in the respective sections of this document) for performing such conductive communication. The communication may thus be provided with an extra layer of security in addition to the encryption by being electrically confined to the conducting path e.g. external device 200, conductive member 201, conductive connection C1, implant 100, meaning the communication will be excessively difficult to be intercepted by a third party not in physical contact with, or at least proximal to, the patient.

The keys described in this section may in some embodiments be generated based on data sensed by sensors described herein under the twelfth or thirteenth aspect, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys. A computer program product of, or adapted to be run on, an external device is also provided, which comprises a computer-readable storage medium with instructions adapted to make the external device perform the actions as described above.

### Aspect 246SE 3-Part Key—Multi-Party Encrypted Communication Between Implant and External Device—Embodiments of Aspect 246SE of the Disclosure

In aspect 246SE, increased security for communication between an external device(s) and an implant is provided. FIGS. 18-29 shows embodiments of this aspect.

First embodiments of aspect 246SE will now be described in conjunction with FIGS. 19-21 and 22. In these embodiments, a method for communication between an external device 200 and an implant is provided. The implant 100 is implanted in a patient and the external device 200 is positioned external to the body of the patient. The implant and the external device each comprise a wireless transceiver 108, 208 for wireless communication W1 between the implant 100 and the external device 200. The wireless transceiver 108 (included in a communication unit 102 of the implant) may in some embodiments comprise sub-transceivers for receiving data from the external device 200 and other external devices 300, 400, 500, e.g. using different frequency bands, modulation schemes etc.

A first step of the method of FIG. 22 comprises receiving S4601, at the implant, by a wireless transmission W1 or otherwise, a first key from an external device 300. The method further comprises receiving S4602, at the implant, by a wireless transmission W1, W2, W3, a second key. The second key may be generated by a second external device, separate from the external device or by another external device 500 being a generator of the second key on behalf of the second external device 300. The second key may be received at the implant from anyone of, the external device 200, the second external device 300, and the generator 500 of the second key. The second external device may be controlled by a caretaker, or any other stakeholder. Said another external device 500 may be controlled by a manufacturer of the implant, or medical staff, caretaker, etc.

In case the implant is receiving the second key from the external device 200, this means that the second key is routed through the external device from the second external device 300 or from the another external device 500 (generator). The routing may be performed as described herein under aspect 253SE. In these cases, the implant and/or external device(s) comprises the necessary features and functionality (described in the respective sections of this document) for performing such routing. Using the external device 200 as a relay, with or without verification from the patient, may provide an extra layer of security as the external device 200 may not need to store or otherwise handle decrypted information. As such, the external device 200 may be lost without losing decrypted information.

The implant comprises a computing unit 106 configured for deriving S4604a a combined key by combining the first key and the second key with a third key held by the implant 100, for example in memory 107 of the implant. The combined key may be used for decrypting S4606, by the computing unit 106, encrypted data transmitted S4605 by a wireless transmission W1 from the external device 200 to the implant 100. Optionally, the decrypted data may be used for altering S4608, by the computing unit 106 an operation of the implant. The altering an operation of the implant may comprise controlling or switching an active unit 101 of the implant. In some embodiments, as described in FIG. 26, the method further comprises at least one of the steps S4640 of, based on the decrypted data, updating a control program running in the implant, and operating the implant 100 using operation instructions in the decrypted data.

In some embodiments, further keys are necessary to derive a combined key for decrypting the encrypted data received at the implant 100. Such embodiments are described in FIG. 23. In these embodiments, the first and second key are received S4601, S4062 as described in conjunction with FIG. 22. Further, the method comprises receiving S4603, at the implant, a fourth key from a third external device 400, the third external device being separate from the external device, deriving S4604b a combined key by combining the first, second and fourth key with the third key held by the implant 100, and decrypting S4606 the encrypted data, in the implant 100, using the combined key. Optionally, the decrypted data may be used for altering S4608, by the computing unit 106, an operation of the implant as described above. In some embodiments, the fourth key is routed through the external device from the third external device. The routing may be performed as described herein under aspect 253SE. In these cases, the implant and/or external device(s) comprises the necessary features and functionality (described in the respective sections of this document) for performing such routing.

In some embodiments, further security measures are needed before using the decrypted data for altering S4608, by the computing unit 106, an operation of the implant. For example, an electrical connection C1 between the implant and the external device, using the body as a conductor, may be used for further verification of validity of the decrypted data. Such embodiments are described in e.g. FIGS. 18-19 and FIG. 24. The electrical connection C1 may be achieved by placing a conductive member 201, configured to be in connection with the external device, in electrical connection with a skin of the patient for conductive communication C1 with the implant. This feature may be achieved as described herein under aspect 247SE. In these cases, the implant and/or external device(s) comprises the necessary features and functionality (described in the respective sections of this document) for performing such conductive communication. The communication may thus be provided with an extra layer of security in addition to the encryption by being electrically confined to the conducting path e.g. external device 200, conductive member 201, conductive connection C1, implant 100, meaning the communication will be excessively difficult to be intercepted by a third party not in physical contact with, or at least proximal to, the patient.

Accordingly, in some embodiments, the method comprising confirming S4607 the electrical connection between the implant and the external device, and as a result of the confirmation, altering S4608 an operation of the implant based on the decrypted data. The confirmation and authentication of the electrical connection may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

Some of these embodiments will now be described for convenience of the reader in conjunction with FIG. 25. In these embodiments, the confirmation S4607 of the electrical connection comprises: measuring S4612 a parameter of the patient, by the implant, measuring S4613 the parameter of the patient, by the external device, comparing S4614 the parameter measured by the implant to the parameter measured by the external device, and authenticating S4615 the connection based on the comparison. As mentioned above, as a result of the confirmation, an operation of the implant may be altered S4608 based on the decrypted data.

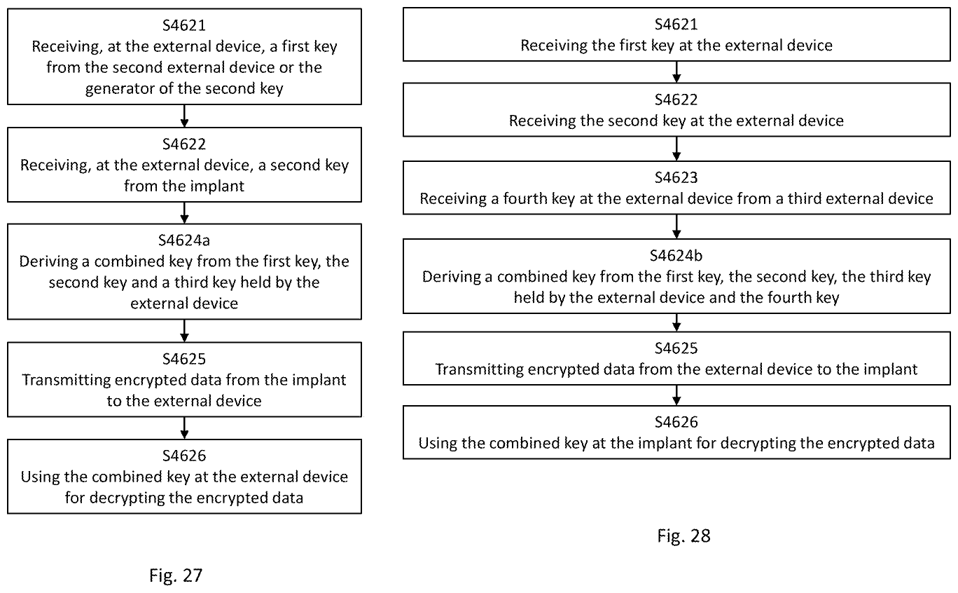

It should be noted that the above concepts of aspect 246SE may be used also for reducing the risk that data transmitted from the implant ends up in the wrong hands. Such embodiments are described in FIGS. 27-28. In the embodiments described in FIG. 27, methods for encrypted communication between an external device 200 and an implant 100 are provided. These methods comprise:
receiving S4621, at the external device 100 by a wireless receiver 208, a first key, the first key being generated by a second external device 300, separate from the external device 200 or by another external device 500 being a generator of the second key on behalf of the second external device 200, the first key being received from anyone of the second external device 200 and the generator 500 of the second key,
receiving S4622, at the external device 200 by the wireless receiver 208, a second key from the implant 100,
deriving S4624a a combined key, by a computing unit 206 of the external device 200, by combining the first key and the second key with a third key held by the external device 200 (e.g. in memory 207),
transmitting S4625 encrypted data from the implant to the external device and receiving the encrypted data at the external device by the wireless receiver 208, and
decrypting S4626, by the computing unit 206, the encrypted data, in the external device 200, using the combined key.

As described above, further keys may be necessary to decrypt the data. Consequently, as described in FIG. 28, the wireless transceiver 208 is configured for:
receiving S4603 a fourth key from a third external device 400,
wherein the computing unit 206 is configured for:
deriving S4604b a combined key by combining the first, second and fourth key with the third key held by the external device, and
decrypting the encrypted data using the combined key.

In some embodiments, the communication between the implant 100 and the external device 200 needs to be confirmed (authenticated) before decrypting the data. The confirmation of the communication may be implemented similar to what is described above, and consequently also as described herein under the fifth, thirteenth and fifteenth aspect. In these cases, the implant and/or external device(s) comprises the necessary features and functionality (described in the respective sections of this document) for performing such authentication.

These embodiments further increase the security in the communication. These embodiments are exemplified in FIG. 29 where the computing unit 206 is configured to confirm the communication between the implant and the external device, wherein the confirmation comprises:
measuring a parameter of the patient, by the external device,
receiving a measured parameter of the patient, from the implant,
comparing the parameter measured by the implant to the parameter measured by the external device,
performing confirmation of the connection based on the comparison, and
as a result of the confirmation, decrypting the encrypted data, in the external device, using the combined key.

The external device 200 may be a wearable external device.

The external device 200 may be a handset.

The second/third external device 300 may be is a handset.

The second/third external device 300 may be a server.

The second/third external device 300 may be cloud based.

One or more of the first, second and third key may comprise a biometric key.

The keys described in this section may in some embodiments be generated based on data sensed by sensors described herein under the twelfth or thirteenth aspect, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

The first key may be received at the implant 100 from the external device 200, by a wireless transmission.

The first key may be transmitted by the external device 200.

The encrypted data may be received from the external device 200 or the second external device or another external device via the internet.

The third external device 300 may be a server comprising a database, the database comprising data pertaining to control program updates and/or instructions. The server may be a device with computing capacity.

The database may communicate with a caregiver and/or the implant 100.

The database may communicate with a caregiver and/or the implant 100 via the external device 200.

The implant 100 may comprises at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

### Aspect 247SE Electrical Connection—Conductive Member in Electrical Connection with the External Device—Embodiments of Aspect 247SE of the Disclosure

In aspect 245SE, increased security for communication between an external device(s) and an implant is provided. FIGS. 30-35 shows embodiments of this aspect.

FIGS. 30-31 shows a system for communication between an external device 200 and an implant 100 implanted in a patient. The system comprises a conductive member 201 configured to be in connection (electrical/conductive or wireless or otherwise) with the external device, the conductive member 201 being configured to be placed in electrical connection with a skin of the patient for conductive communication C1 with the implant 100. By using a conductive member 201 as defined herein, an increased security for communication between the external device and the implant may be achieved. For example, when a sensitive update of a control program of the implant 100 is to be made, or if sensitive data regarding physical parameters of the patient is to be sent to the externa device 200 (or otherwise), the conductive member 201 may ensure that the patient is aware of such communication and actively participate in validating that the communication may take place. The conductive member may, by being placed in connection with the skin of the patient, open the conductive communication channel C1 between the external device and the implant to be used for data transmission.

Electrical or conductive communication, such as this or as described under the other aspects, may be very hard to detect remotely, or at least relatively so, in relation to wireless communications such as radio transmissions. Direct electrical communication may further safeguard the connection between the implant and the external device from electromagnetic jamming i.e. high-power transmissions other a broad range of radio frequencies aimed at drowning other communications within the frequency range. Electrical or conductive communication will be excessively difficult to be intercepted by a third party not in physical contact with, or at least proximal to, the patient, providing an extra level of security to the communication.

In some embodiments, the conductive member comprises a conductive interface for connecting the conductive member to the external device.

The conductive interface may be any suitable hardware interface, such as a charging port of the external device, a headphone port, a USB port, a serial port, an ethernet port, a DVI port, printer port etc. By using a hardware port for connection, the security aspect is further facilitated since it may be ensured that both the external device and the conductive member are present near the patient and connected using a wired interface.

In other embodiments, the conductive member is wirelessly connected to the external device using a wireless communication channel (Radio), such as WLAN, W1-Fi, cellular network, Bluetooth, NFC, RFID etc.

In some embodiments, the conductive member 201 is a device which is plugged into the external device 200, and easily visible and identifiable for simplified usage by the patient. In other embodiments, the conductive member 201 is to a higher degree integrated with the external device 200, for example in the form of a case 201a of the external device 200, the case 201a comprising a capacitive area configured to be in electrical connection with a skin of the patient. In FIG. 31, the case 201a is exemplified as a mobile phone case (smartphone case) for a mobile phone, but the case may in other embodiments be a case for a personal computer, or a body worn camera, or any other suitable type of external device as described herein. The case may for example be connected to the phone using a wire from the case and connected to the headphone port or charging port of the mobile phone.

The conductive communication C1 may be used both for communication between the implant and the external device 200 in any or both directions. Consequently, according to some embodiments, the external device 200 is configured to transmit a conductive communication (conductive data) to the implant 100 via the conductive member 201.

According to some embodiments, the implant 100 is configured to transmit a conductive communication to the external device 200. The content of the conductive communication is exemplified in FIG. 32. These embodiments start by placing S4701 the conductive member 201, configured to be in connection with the external device, in electrical connection with a skin of the patient for conductive communication C1 with the implant. The conductive communication between the external device 200 and the implant 100 may follow an electrically/conductively confined path comprising e.g. the external device 200, conductive member 201, conductive connection C1, implant 100.

For the embodiments when the external device 200 transmits data to the implant, the communication may comprise transmitting S4704a a conductive communication to the implant 100 by the external device 200.

The transmitted data may comprise instructions for operating the implant. Consequently, some embodiments comprise operating S4730 the implant 100 using operation instructions, by an internal computing unit 106 of the implant 100, wherein the conductive communication C1 comprises instructions for operating the implant. The operation instruction may for example involve adjusting or setting up (e.g. properties or functionality of) an active unit 101 of the implant.

The transmitted data may comprise instructions for updating a control program 110 stored in memory 107 of the implant 100. Consequently, some embodiments comprise updating S4740 the control program 110 running in the implant, by the internal computing unit 106 of the implant, wherein the conductive communication comprises instructions for updating the control program.

For the embodiments when the implant 100 transmits data to the external device 200, the communication may comprise transmitting S4704b conductive communication C1 to the external device by the implant 100. The conductive communication may comprise feedback parameters (battery status, properties, version number etc.) relating to functionality of the implant. In other embodiments, the conductive communication C1 comprises data pertaining to least one physiological parameter of the patient, such as blood pressure etc. The physiological parameter(s) may be stored in memory 107 of the implant 100 or sensed in prior (in real time or with delay) to transmitting S4704b the conductive communication C1. Consequently, in some embodiments, the implant comprises a sensor 150 for sensing S4750 at least one physiological parameter of the patient, wherein the conductive communication comprises said at least one physiological parameter of the patient.

To further increase security of the communication between the implant 100 and the external device 200, different types of authentication, verification and/or encryption may be employed. In some embodiments, as described in FIG. 33 in conjunction with FIG. 31, the external device 200 comprises a verification unit 220. The verification unit may be any type of unit suitable for verification of a user, i.e. configured to receive authentication input from a user, for authenticating the conductive communication between the implant and the external device. In some embodiments, the verification unit and the external device comprises means for collecting authentication input from the user (which may or may not be the patient). Such means may comprise a fingerprint reader, a retina scanner, a camera, a GUI for inputting a code, a microphone, device configured to draw blood, etc. The authentication input may thus comprise a code or any be based on a biometric technique selected from the list of: a fingerprint, a palm vein structure, image recognition, face recognition, iris recognition, a retinal scan, a hand geometry, and genome comparison. The means for collecting the authentication input may alternatively be part of the conductive member which comprise any of the above examples of functionality, such as a fingerprint reader 222 or other type of biometric reader 222.

In some embodiment, as exemplified in FIG. 33, the security may thus be increased by receiving S4702 an authentication input from a user by a verification unit 220 of the external device, and authenticating S4703 the conductive communication between the implant and the external device using the authentication input. Upon a positive authentication S4703, the conductive communication channel C1 may be employed for comprising transmitting a S4704a conductive communication to the implant by external device 200 and/or transmitting S4704b a conductive communication to the external device 200 by the implant 100. In other embodiments, a positive authentication is needed prior to operating S4730 the implant based on received conductive communication, and/or updating S4740 a control program running in the implant as described above.

Other ways of performing authentication of the conductive communication are equally possible. For example, as exemplified in FIG. 34, secure communication may be achieved by the implant comprising: a sensor 150 (e.g. connected through a wire C3, or wirelessly connected, to the implant) for measuring S4712 a parameter of the patient, by the implant, and an internal computing unit 106 configured for:
i. receiving S4713 a parameter of the patient, from the external device 200 (via conductive communication C1 or via a wireless communication W1),
ii. comparing S4714 the parameter measured by the implant 100 to the parameter measured by the external device, and
iii. performing S4715 authentication of the conductive communication based on the comparison.

In other embodiments, the implant 100 being connected to a sensation generator 181 (included in the implant or separate from the implant), the implant being configured for: storing authentication data (in memory 107), related to a sensation generated by the sensation generator,
receiving input authentication data from the external device 200. The implant 100 comprises an internal computing unit 106 configured for:
i. comparing the authentication data to the input authentication data, and
ii. performing authentication of the conductive communication based on the comparison.

Upon a positive authentication, the conductive communication channel C1 may be employed for comprising transmitting a S4704a conductive communication to the implant 100 by external device 200 and/or transmitting S4704b a conductive communication to the external device 200 by the implant 100. In other embodiments, a positive authentication is needed prior to operating S4730 the implant based on received conductive communication, and/or updating S4740 a control program running in the implant as described above.

The confirmation and authentication of the conductive communication (electrical connection) may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication and communication may be increased as it may require a malicious third party to know or gain access to either the transient physiological parameter of the patient or detect randomized sensations generated at or within the patient in addition to accessing the implant via a conductive path.

In other embodiments, the conductive communication comprises a key or a part of the key to be used for decrypting encrypted data received by the external device or the implant. Such embodiment is exemplified in FIG. 35. This embodiment starts by placing S4701 the conductive member 201, configured to be in connection with the external device, in electrical connection with a skin of the patient for conductive communication C1 with the implant. The external device is configured to transmit S4722 a first part of the key to the implant 100 using the conductive communication C1, and to wirelessly W1 transmit a second part of the key to the implant 100, wherein the implant 100 (e.g. the computing unit 106) is adapted to decrypt S4725 the encrypted data, using a combined S4724 key derived from the received first and second parts of the key. Wireless communication may be achieved by wireless transceivers 108, 208 of the implant 100 and the external device 200. Further examples and details of how to perform encryption of data transmitted between the implant 100 and the external device 200 can be found as described herein under the second, third or sixth aspect. In these cases, the implant and/or external device(s) comprises the necessary features and functionality (described in the respective sections of this document) for performing such encryption/decryption.

The conductive member may be arranged as an arm or wrist band being integrally formed with, or connected to, the external device. The arm or wrist band may be configured to be worn, around an arm or wrist, of the patient in which the implant is implanted.

The conductive member may be configured to be in conductive or electrical connection with the external device.

The conductive member may be configured to be in wireless connection with the external device.

The conductive member may be configured to be a screen of the external device, the screen being configured to receive data using electric charge.

The conductive member may comprise the verification unit.

The external device may comprise the verification unit.

The establishment of conductive communication may be configured to authenticate or partially authenticate the conductive communication between the implant and the external device.

The external device may be a smartwatch. The smartwatch may be configured to be worn, around an arm or wrist, of the patient in which the implant is implanted. The smartwatch may additionally function as a mobile computing and communication device or a device for displaying the time, i.e. a clock.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

### Aspect 248SE Device Synchronization Sensation—Authenticating a Connection Between an Implant and the External Device Using Sensations—Embodiments of Aspect 248SE of the Disclosure

FIGS. 36, 37, and 38 show an implant 100 implanted in a patient and an external device 200. The figures further show the implant 100 being connected to a sensation generator 181.

The sensation generator 181 may be configured to generate a sensation. The sensation generator may be contained within the implant 100 or be a separate unit. The sensation generator 181 may be implanted. The sensation generator 181 may also be located so that it is not implanted as such but still is in connection with a patient so that only the patient may experience sensations generated. The implant is configured for storing authentication data, related to the sensation generated by the sensation generator 181.

The implant 100 is further configured for receiving input authentication data from the external device 200. Authentication data related to the sensation generated may by stored by a memory 107 of the implant 100. The authentication data may include information about the generated sensation such that it may be analyzed, e.g. compared, to input authentication data to authenticate the connection, communication, or device. Input authentication data relates to information generated by a patient input to the external device 200. The input authentication data may be the actual patient input or an encoded version of the patient input, encoded by the external device 200. Authentication data and input authentication data may comprise a number of sensations or sensation components.

The authentication data may comprise a timestamp. The input authentication data may comprise a timestamp of the input from the patient. The timestamps may be a time of the event such as the generation of a sensation by the sensation generator 181 or the creation of input authentication data by the patient. The timestamps may be encoded. The timestamps may feature arbitrary time units, i.e. not the actual time. Timestamps may be provided by an internal clock 160 of the implant 100 and an external clock 260 of the external device. The clocks 160, 260 may be synchronized with each other. The clocks 160, 260 may be synchronized by using a conductive connection C1 or a wireless connection W1 for communicating synchronization data from the external device 200, and its respective clock 260, to the implant 100, and its respective clock 160, and vice versa. Synchronization of the clocks 160, 260 may be performed continuously and may not be reliant on secure communication.

Authentication of the connection may comprise calculating a time difference between the timestamp of the sensation and the timestamp of the input from the patient, and upon determining that the time difference is less than a threshold, authenticating the connection. An example of a threshold may be is. The analysis may also comprise a low threshold as to filter away input from the patient that is faster than normal human response times. The low threshold may e.g. be 50 ms.

Authentication data may comprise a number of times that the sensation is generated by the sensation generator, and wherein the input authentication data comprises an input from the patient relating to a number of times the patient detected the sensation. Authenticating the connection may then comprise: upon determining that the number of times that the authentication data and the input authentication data are equal, authenticating the connection.

FIG. 36 shows the implant 100 comprising a communication unit 102 which in turn may comprise an internal computing unit 106 and the memory 107. The internal computing unit 106 may be configured for analyzing the authentication data and the input authentication data and performing authentication of the connection based on the analysis. The internal computing unit 106 may form integrally a part of the communication unit 102, as shown, or be a separate unit of the implant 100.

The external device 200, adapted for connection with the implant, may comprise an interface for an input from the patient resulting in input authentication data. This interface may e.g. comprise an electrical switch, a biometric input sensor or a digital interface running on the external device 200 to name just a few examples. A biometric sensor may provide an extra level of authentication as the identity of the patient may be verified by providing input authentication. A digital interface may also provide an extra level of authentication by requiring for example input of a pass code known by the patient. An example of the biometric input sensor would be a fingerprint reader.

The external device 200 may further comprise a receiver for receiving the authentication data from the implant, the authentication data relating to a generated sensation of the sensation generator 181 connected to the implant 100. The receiver may be a transceiver 208 of the external device 200 or a separate unit. The external device 200 may further comprise an external computing unit 206. The external computing unit 206 may be configured for analyzing the authentication data to the input authentication data and performing authentication of the connection based on the analysis.

FIG. 36 further shows the implant 100 being in wireless W1 or conductive C1 communication with the external device 200. The connections may be used for communicating further data from the implant 100 to the external device 200 following positive authentication and vice versa. Further data may be communicated between the implant 100 and the external device 200 following positive authentication.

FIG. 38 shows the implant 100 comprising a motor 183. The motor may be adapted for controlling a physical function in the body of the patient. The motor 183 may be related to the active device 101 of the implant 100. The motor may further function as the sensation generator 181. The motor may be used to generate the sensation. The motor 183 may specifically be adapted to generate a vibration or a sound by running or operating the motor 183. FIG. 38 further shows the implant 100 comprising a sensor 150, connected to the implant. The sensor 150 may be comprised within the implant or be a separate unit.

The conductive connection or communication C1 discussed herein may be routed between the external device 200 and the implant 100 via a conductive member 201. Features of such communication are a subject of aspect 247SE. The communication may thus be provided with an extra layer of security by being electrically confined to the conducting path e.g. external device 200, conductive member 201, conductive connection C1, implant 100, meaning the communication will be excessively difficult to be intercepted by a third party not in physical contact with, or at least proximal to, the patient. Using the conductive connection C1 for communication input authentication data and authentication data related to.

FIG. 39 shows a schematic flow chart of the steps for the method of authenticating the connection between an implant 100 implanted in a patient, and an external device 200 according to the first part of aspect 248SE. The method includes the following steps.

Generating S4801, by a sensation generator 181, a sensation detectable by a sense of the patient. The sensation may comprise a plurality of sensation components. The sensation or sensation components may comprise a vibration, a sound, a photonic signal, a light signal, an electric signal, or a heat signal.

A vibration may comprise a single or sequence of vibrations of at least one frequency. A sound may be an audible sound with a frequency in the range 20-20000 Hz. A sound may comprise a plurality or a sequence of fixed frequency vibrations. Such a signal may be audible to an ear of the patient. Vibrations and sounds may also be configured to be registered by tactile, pressure, and pain receptors of the patient.

A photonic signal may be any electromagnetic wave-based signal such as a radio wave signal or an infrared light signal within the infrared wavelength range 700-1 mm. A light signal may comprise a signal based on visible light pulses in the wavelength range from 380-750 nm. A light signal is more preferably in the red sub-range of visible light i.e. >600 nm. Light signals may be visible to an eye of the patient. In general, longer wavelength photonic and light signals may be preferable for longer tissue penetration depths. An infrared signal may be configured to be visible by an infrared sensor or camera external to the body of the patient.

An electric signal may comprise a faint electric pulse configured to be felt by the patient. The power of the electric signal may be configured with feedback to increase until the signal is felt by the patient. Such a signal may be configured to be felt by pain receptors of the patient. A heat signal may comprise a thermodynamic signal with higher and lower temperature pulses. Such a signal may be configured to be felt by thermal receptors or pain receptors of the patient. A heat signal may be created by a thermal element.

Sensations may be configured to be consistently felt by a sense of the patient while not risking harm to or affecting internal biological processes of the patient.

The sensation generator 181, may be contained within the implant 100 or be a separate entity connected to the implant 100. The sensation may be generated by a motor 183 of the implant 100 for controlling a physical function in the body of the patient, wherein the motor being the sensation generator 181. The sensation may be a vibration, or a sound created by running the motor 183. The sensation generator 181 may be located close to a skin of the patient and thus also the sensory receptors of the skin. Thereby the strength of some signal types may be reduced.

Storing S4802, by the implant 100, authentication data, related to the generated sensation.

Providing S4803, by the patient input to the external device, resulting in input authentication data. Providing the input may e.g. comprise an engaging an electrical switch, using a biometric input sensor or entry into digital interface running on the external device 200 to name just a few examples.

Transmitting S4806a the input authentication data from the external device to the implant 100. If step S4806a was performed, the analysis may be performed by the implant 100.

Transmitting S4806b the authentication data from the implant 100 to the external device 200. If step S4806b was performed, the analysis may be performed by the external device 200. The wireless connection W1 or the conductive connection C1 may be used to transmit the authentication data or the input authentication data of steps S4806a and S4806b.

Authenticating S4804 the connection based on an analysis of the input authentication data and the authentication data e.g. by comparing a number of sensations generated and experienced or comparing timestamps of the authentication data and the input authentication data. If step S4806a was performed, the analysis may be performed by the implant 100.

Communicating S4805 further data between the implant and the external device following positive authentication. The wireless connection W1 or the conductive connection C1 may be used to communicate the further data. The further data may comprise data for updating a control program 110 running in the implant 100. or operation instructions for operating the implant 100. The further data may also comprise data sensed by a sensor 150 connected to the implant 100.

If the analysis was performed by the implant 100, the external device 200 may continuously request or receive S4808a, information of an authentication status of the connection between the implant and the external device 200, and upon determining S4809a, at the external device 200, that the connection is authenticated, transmitting S4805 further data from the external device 200 to the implant 100.

If the analysis was performed by the external device 200, the implant 100 may continuously request or receive S4808b, information of an authentication status of the connection between the implant and the external device 200, and upon determining S4809b, at the implant 100, that the connection is authenticated, transmitting S4805 further data from the implant 100 to the external device 200.

A main advantage of authenticating a connection according to this fifth aspect is that only the patient may be able to experience the sensation. Thus, only the patient may be able to authenticate the connection by providing authentication input corresponding to the sensation generation.

The sensation generator 181, sensation, sensation components, authentication data, input authentication data, and further data may be further described herein under aspect 257SE. In these cases, the implant and/or external device(s) comprises the necessary features and functionality (described in the respective sections of this document). Further information and definitions can be found in this document in conjunction with the other aspects.

The method may further comprise transmitting further data between the implant and the external device, wherein the further data is used or acted upon, only after authentication of the connection is performed.

The analysis or step of analyzing may be understood as a comparison or a step of comparing.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

### Aspect 249SE Prior Verified Communication—Verifying Authenticity of Instructions Sent from the External Device to the Implant—Embodiments of Aspect 249SE of the Disclosure

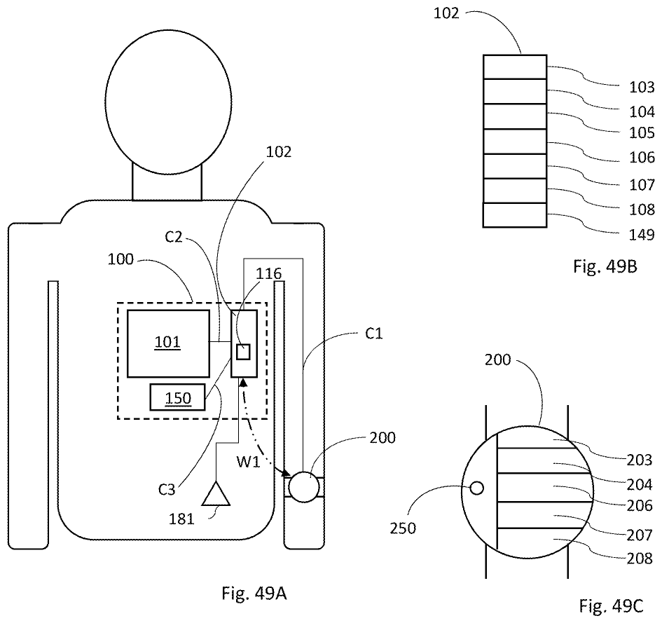

In aspect 249SE, increased security for communication between an external device(s) and an implant is provided. FIGS. 40-50 shows embodiments of this aspect. FIG. 49A-C shows embodiments of an implant 100, a communication unit 102 and an external device 200 which may form a system.

The implant 100 comprises a transceiver 108, 103 configured to establish a connection with an external device 200, i.e. with a corresponding transceiver 208, 203. The connection may be an electrical connection C1 using the transceivers 103, 203, or a wireless connection W1 using the transceivers 108, 208. The implant further comprising a computing unit 106 configured to verify the authenticity of instructions received at the transceiver 108, 103 from the external device 200. In this aspect, the concept of using previously transmitted instructions for verifying a currently transmitted instructions are employed. Consequently, the transmitting node (in this case the external device) need to be aware of previously instructions transmitted to the implant, which reduces the risk of a malicious device instructing the implant without having the authority to do so.

FIG. 40 shows one embodiment of verifying the authenticity of instructions received at the implant, the embodiment relating to communicating instructions from an external device 200 to an implant 100 implanted in a patient, using an established S4910 connection between the external device and the implant 100. The connection may be a conductive communication link, or a wireless communication link.

In this embodiment, the computing unit 106 is configured to verify the authenticity of instructions received at the transceiver 108, 103 by extracting a previously transmitted set of instructions from a first combined set of instructions received by the transceiver. The external device 200 may thus comprise an external device comprising a computing unit 206 configured for: combining a first set of instructions with a previously transmitted set of instructions, forming a combined set of instructions, and transmitting the combined set of instructions to the implant. The previously transmitted set of instructions, or a representation thereof, may be stored in memory 207 of the external device 200.

The combined set of instructions may have a data format which facilitates such extraction, for example including metadata identifying data relating to the previously transmitted set of instructions in the combined set of instructions. In some embodiments, the combined set of instructions comprises the first set of instructions and a cryptographic hash of the previously transmitted set of instructions. Consequently, the method comprises combining S4920, at the external device, a first set of instructions with a previously transmitted set of instructions, forming a first combined set of instructions. A cryptographic hash function is a special class of hash function that has certain properties which make it suitable for use in cryptography. It is a mathematical algorithm that maps data of arbitrary size to a bit string of a fixed size (a hash) and is designed to be a one-way function, that is, a function which is infeasible to invert. Examples include MD 5, SHA 1, SHA 256, etc. Increased security is thus achieved.

The first combined set of instructions is then transmitted S4930 to the implant 100, where it is received by e.g. the transceiver 103, 108. The first combined set of instructions may be transmitted to the implant using a proprietary network protocol. The first combined set of instructions may be transmitted to the implant using a standard network protocol. More embodiments describing network protocols may be implemented as described herein under aspect 250SE. In these cases, the implant and/or external device(s) comprises the necessary features and functionality (described in the respective sections of this document) for performing transmission of data. By using different communication protocols, at the external device 200, for communication to the implant 100 and a second external device 300, as described in aspect 250SE, an extra layer of security is added as the communication between implant and the external device may be made less directly accessible to remote third parties.

At the implant 100, the computing unit verifies S4930 the authenticity of the received first combined set of instructions, by: extracting S4941 the previously transmitted set of instructions from the first combined set of instructions, and comparing S4942a the extracted previously transmitted set of instructions with previously received instructions stored in the implant.

Upon determining that the extracted previously transmitted set of instructions equals the previously received instructions stored in the implant, the authenticity of the received first combined set of instructions may be determined as valid, and consequently, the first set of instructions may be safely run S4943a at the implant, and the first combined set of instructions may be stored in memory 107 of the implant, to be used for verifying a subsequent received set of instructions.

In some embodiments, upon determining by the internal computing unit 106 that the extracted previously transmitted set of instructions differs from the previously received instructions stored in the implant, feedback related to an unauthorized attempt to instruct the implant may be provided S4943b. For example, the transceiver 108, 103 may send out a distress signal to e.g. the external device 200 or to any other connected devices. The implant 100 may otherwise inform the patient that something is wrong by e.g. vibration or audio. The implant 100 may be run in safe mode, using a preconfigured control program which is stored in memory 107 and specifically set up for these situations, e.g. by requiring specific encoding to instruct the implant, or only allow a predetermined device (e.g. provided by the manufacturer) to instruct the implant 100. In some embodiments, when receiving such feedback at the external device 200, the external device 200 retransmits S4930 the first combined set of instructions again, since the unauthorized attempt may in reality be an error in transmission (where bits of the combined set of instructions are lost in transmission), and where the attempt to instruct the implant is indeed authorized.

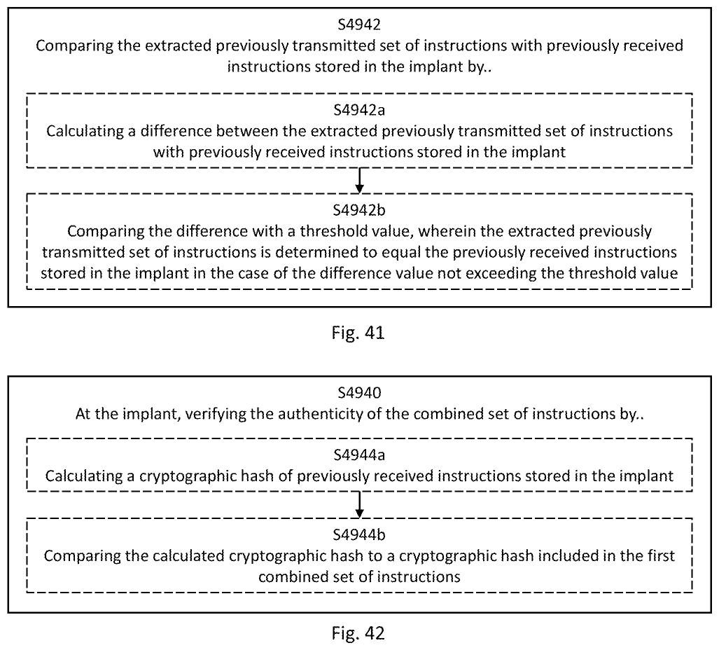

The step of comparing S4942 the extracted previously transmitted set of instructions with previously received instructions stored in the implant may be done in different ways. For example, as shown in FIG. 41 the step of comparing S4942 the extracted previously transmitted set of instructions with previously received instructions stored in the implant comprises calculating S4942a a difference between the extracted previously transmitted set of instructions with previously received instructions stored in the implant, and comparing S4942b the difference with a threshold value, wherein the extracted previously transmitted set of instructions is determined to equal the previously received instructions stored in the implant in the case of the difference value not exceeding the threshold value. This embodiment may be used when received instructions is stored in clear text, or a representation thereof, in the implant, and where the combined set of instructions, transmitted S4930 from the external device also includes such a representation of the previously transmitted instructions. This embodiment may be robust against error in transmission where bits of information are lost or otherwise scrambled.

In other embodiments, shown in FIG. 42, the combined set of instructions comprises the first set of instructions and a cryptographic hash of the previously transmitted set of instructions, wherein the method further comprises, at the implant, calculating S4944a a cryptographic hash of the previously received instructions stored in the implant and comparing S4944b the calculated cryptographic hash to the cryptographic hash included in the first combined set of instructions. This embodiment provides increased security since the cryptographic hash is difficult to decode or forge.

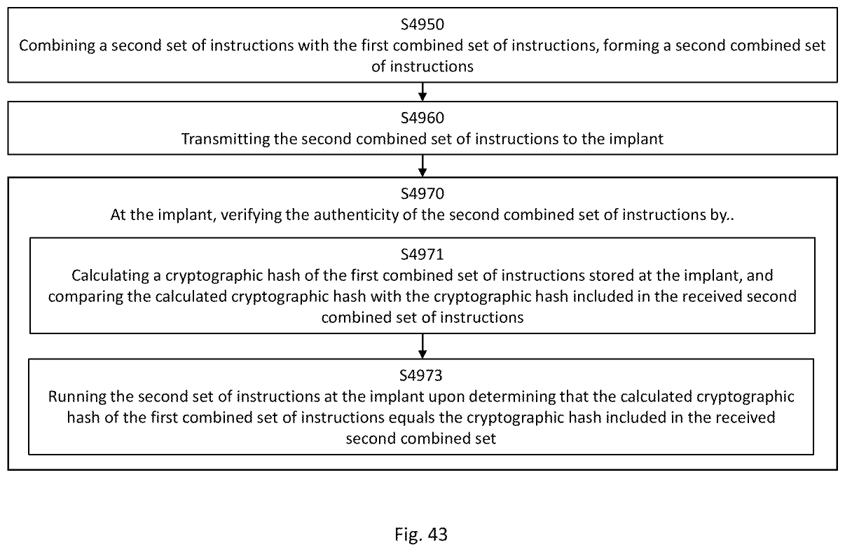

As shown in FIG. 43, the above way of verifying the authenticity of received instructions at the implant may be iteratively employed for further sets if instructions. These embodiments comprise combining S4950, at the external device 200, a second set of instructions with the first combined set of instructions, forming a second combined set of instructions, wherein the second combined set of instructions comprises a cryptographic hash of the first combined set of instructions. The second combined set of instructions is transmitted S4960 to the implant 100. At the implant, the authenticity of the second combined set of instructions may be verified S4970 by: calculating S4971 a cryptographic hash of the first combined set of instructions stored in the implant, and comparing the calculated cryptographic hash with the cryptographic hash included in the received second combined set of instructions, and upon determining that the calculated cryptographic hash of the first combined set of instructions equals the cryptographic hash included in the received second combined set, running S4973 the second set of instructions at the implant and storing the second combined set of instruction in the implant, to be used for verifying a subsequent received set of instructions. Since the first combined set of instructions stored at the implant comprises the hash of the data of the received previous sets of instructions (above called the previously received instructions stored in the implant, which herein refers to the genesis block, or the originally received first set of instructions at the implant), the security of the authenticity of the received set of instructions may be further increased for each received set of instructions.

To further increase security, the transmission of a first set of instructions, to be stored at the implant 100 for verifying subsequent sets of combined instructions, where each set of received combined instructions will comprise data which in some form will represent, or be based on, the first set of instruction, may be performed according to the following examples.

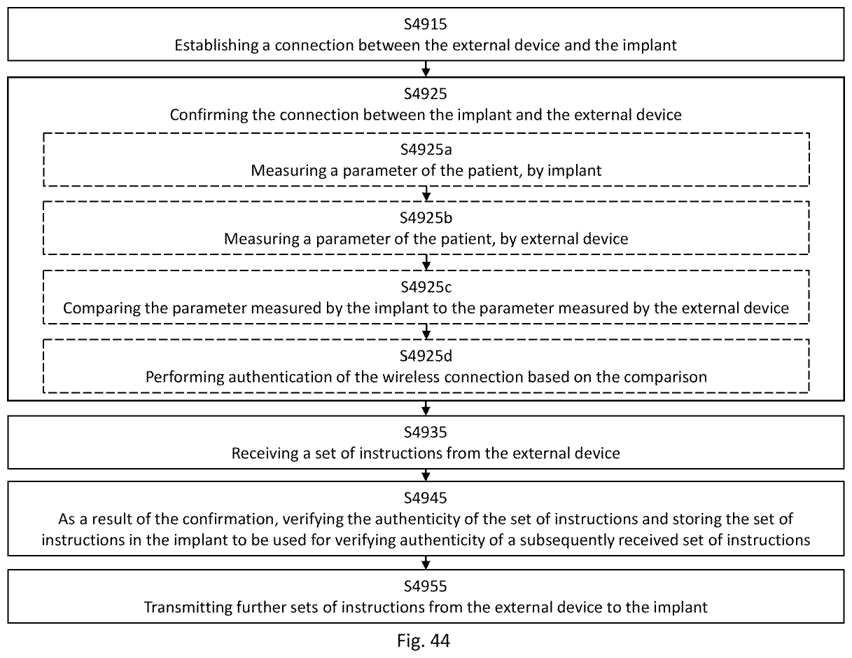

In some embodiments, shown in FIG. 44, method of communicating instructions from the external device 200 to the implant 100 implanted in a patient is disclosed, comprising the steps of: establishing S4915 a connection between the external device and the implant, confirming S4925 the connection between the implant and the external device, receiving S4935 a set of instructions from the external device, as a result of the confirmation, verifying S4945 the authenticity of the set of instructions and storing the set of instructions in the implant to be used for verifying authenticity of a subsequently received set of instructions. The set of instructions may subsequently be included according to the above in a received combined set of instructions, and the set of instructions stored in the implant may be used to verify the authenticity of the combined set of instructions according to the above.

The step of confirming S4925 the connection between the implant and the external device may include: measuring S4925a a parameter of the patient, by implant, measuring S4925b a parameter of the patient, by external device, comparing S4925c the parameter measured by the implant to the parameter measured by the external device, and performing S4925d authentication of the connection based on the comparison. This is shown in FIG. 44.

The step of confirming S4925 the connection between the implant and the external device may include: generating S4926a, by a sensation generator, a sensation detectable by a sense of the patient, storing S4926b, by the implant, authentication data, related to the generated sensation, providing S4926c, by the patient, input to the external device, resulting in input authentication data, authenticating S4926d the connection based on a comparison of the input authentication data and the authentication data. This is shown in FIG. 45.

The confirmation and authentication of the connection may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

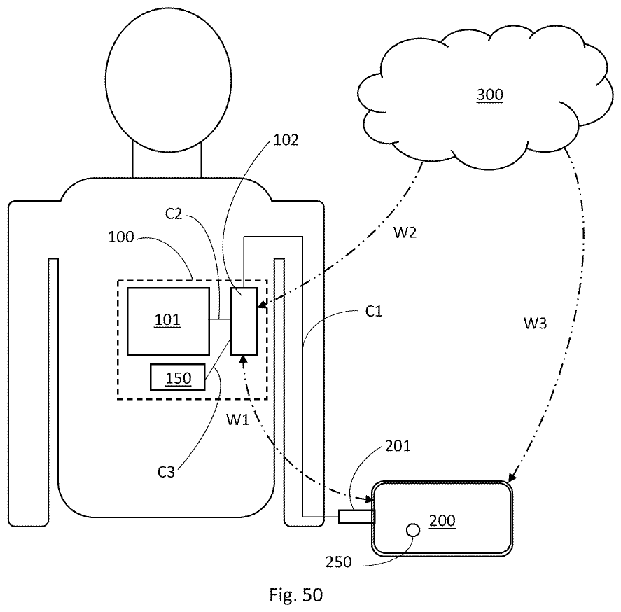

In other embodiments, other ways of increasing the security of the genesis block, i.e. the transmission of a first set of instructions, to be stored at the implant 100 for verifying subsequent sets of combined instructions, may be employed. For example, as shown in FIG. 46, by placing S4951 a conductive member 201 (see FIG. 50), configured to be in connection with the external device 200, in electrical connection C1 with a skin of the patient for conductive communication with the implant, transmitting S4952, via the electrical connection using conductive communication, a set of instructions from the external device, receiving S4953, at the implant the set of instructions from the external device, storing S4954 the set of instructions in the implant to be used for verifying authenticity of a subsequently received set of instructions. Consequently, it may be ensured that the genesis block is transmitted from a device under control of the patient in which the implant is implanted. Optionally, prior to transmitting, via the electrical connection using conductive communication, a set of instructions from the external device, authentication input may be received S4956 from a user by a verification unit 220 of the external device, and the conductive communication between the implant and the external device may be authenticated S4957 using the authentication input. As a result of the authentication, i.e. if the verification is correct/valid (correct code, valid finger print, etc.), the set of instructions may be transmitted S4952, via the electrical connection C1 using conductive communication, from the external device 200 to the implant 100.

Further information and details around the conductive member and other involved devices and processes for achieving a conductive communication may be performed as described herein under aspect 247SE. In these cases, the implant and/or external device(s) comprises the necessary features and functionality (described in the respective sections of this document) for performing such a conductive communication.

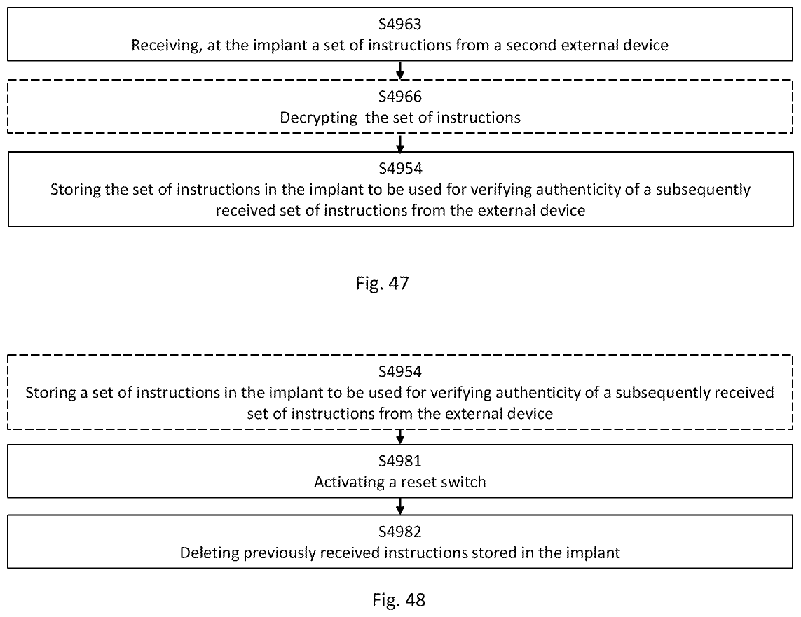

In other embodiments, other ways of increasing security for an authorized transmission of the genesis block may be employed. For example, as shown in FIG. 47, a set of instructions may be received S4963, using a wireless transmission W2, at the implant 100 from a second external device 300, which set of instructions may be stored S4954 in the implant to be used for verifying authenticity of a subsequently received set of instructions from the external device 100. The second external device 300 may be a device with higher trust, such as for example a device under control of the hospital, care taker, manufacturer etc. The second external device 300 may transmit the set of instructions using a proprietary network protocol to further increase security (e.g. as described herein under aspect 250SE). This may improve security of authentication by adding another layer in the communication.

In this embodiment, the external device is configured to receive W3 a set of instructions (e.g. the genesis block) from the second external device 300, store said set of instructions, wherein the external device comprises a computing unit 206 configured to combining a first set of instructions with a said stored set of instructions, thus forming a combined set of instructions, transmitting the combined set of instructions to the implant.

Optionally, the set of instructions received by the implant 100 from the second external device is encrypted, wherein the method further comprising decrypting S4966 the set of instructions and storing S4954 the decrypted set of instructions in the implant to be used for verifying authenticity of a subsequently received set of instructions from the external device.

According to some embodiments, as shown in FIG. 48, a reset function or switch 116 (shown in FIG. 49A) at the implant may be employed to delete S4982 any previously received instructions stored in the implant 100, by being activated S4981. Further information and details around the reset function or switch 116 and other involved devices and processes for handling such reset function or switch 116 may be performed as described herein under the aspect 244SE. In these cases, the implant and/or external device(s) comprises the necessary features and functionality (described in the respective sections of this document) for performing such a resetting of the implant 100. Such a scheme for resetting or deleting instructions from the implant may increase the security of the implant by requiring a physical reset action to be performed. As such, remote resetting, with malicious intent, may be prevented.

Any of the above embodiments for transmitting a “new” genesis block to the implant 100 may subsequently be employed.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

Further information and definitions of features, functionality of this aspect can be found in this document in conjunction with the other aspects.

### Aspect 250SE Dual Protocols—Two Wireless Communication Protocols for Communication—Embodiments of Aspect 250SE of the Disclosure

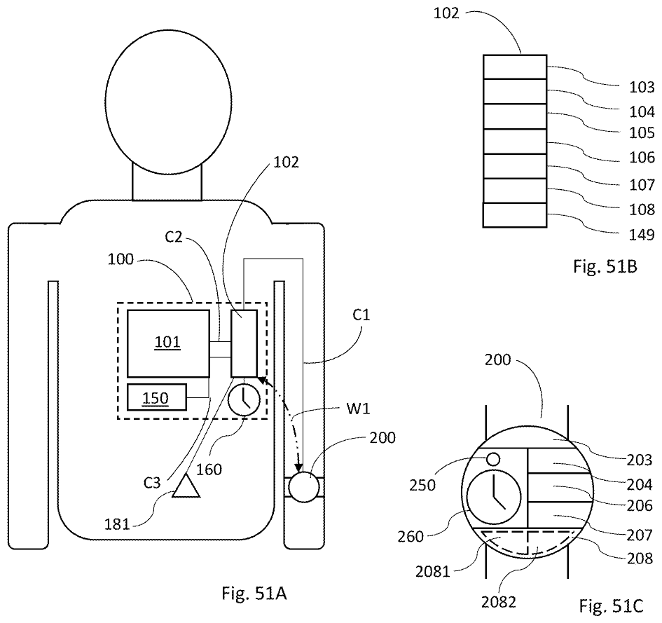

In aspect 250SE, methods and devices and systems for communication between external device(s) and an implant are provided. FIGS. 51-55 show embodiments of this aspect. Generally, the use of standard network protocols for communication between the external devices, and a proprietary network protocol for communication between an external device and the implant provides an increased security for communication in such a system. When limiting communication with the implant to the use of a proprietary network protocol, the risk of a malicious device instructing the implant without having the authority to do so is reduced.

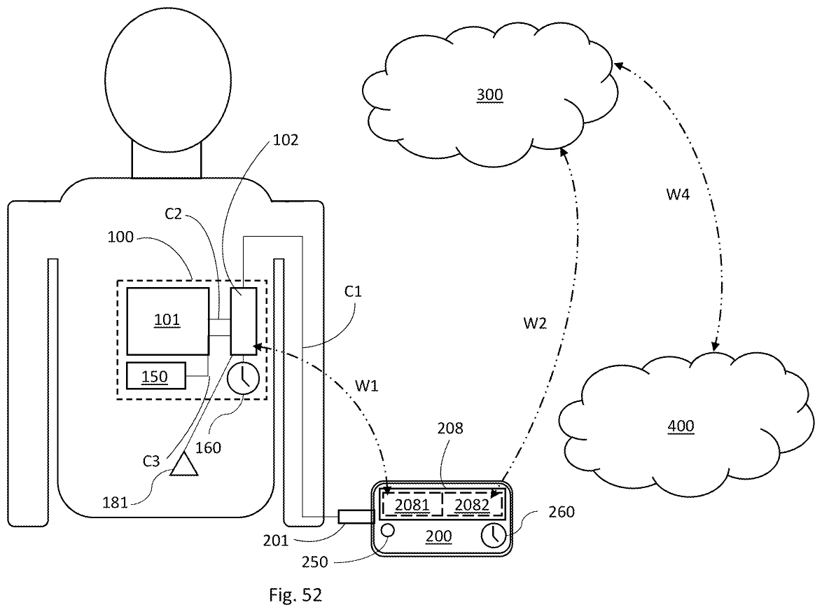

FIG. 51A-C shows example embodiments of an implant 100, a communication unit 102 of an implant 100 and an external device 200 configured to communicate with the implant. FIG. 52 shows embodiments of an implant 100, external devices 200, 300, 400 which may form a system.

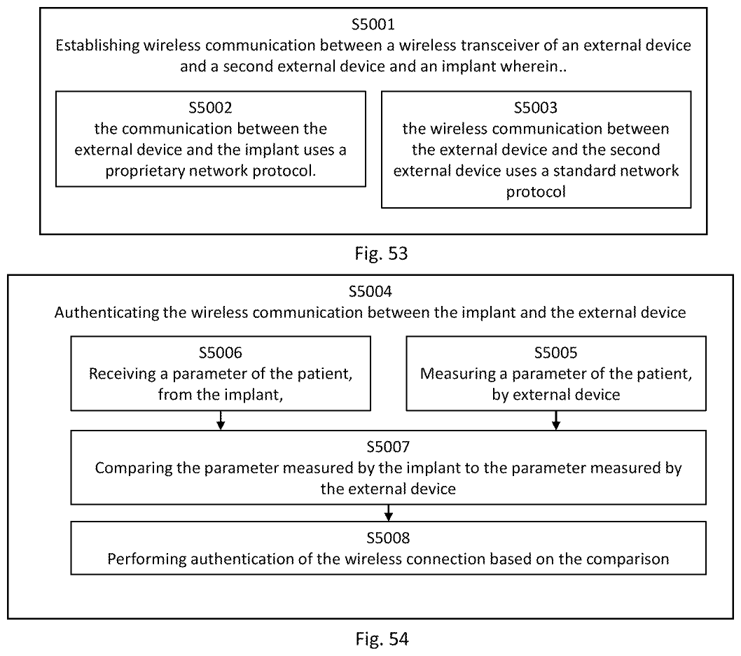

As shown in FIGS. 51C and 52, the external device 200 comprises at least one wireless transceiver 208 configured for wireless communication W1, W2 with the second external device 300 and the implant 100, wherein the wireless transceiver 208 is configured to communicate W1 with the implant using a proprietary network protocol. As shown in FIG. 53, the communication from the external device 200 may comprise establishing S5001 wireless communication W1, W2 between at least one wireless transceiver 208 of an external device 200 and a second external device 300 and the implant 100, wherein the communication W1 between the external device 200 and the implant 100 uses S5002 a proprietary network protocol, and wherein the wireless communication W2 between the external device and the second external device uses S5003 a standard network protocol.

The implant 100 comprises a wireless receiver configured for receiving W1 communication using the proprietary network protocol. The wireless receiver of the implant 100 may be configured for only receiving communication using the proprietary network protocol, for example by having an antenna of the wireless receiver of the implant 100 configured to only receive in a first frequency band, wherein the frequency band of the proprietary network protocol is included in the first frequency band. In other embodiments, a computing unit 106 of the implant may be configured to discard any communication received by the implant 100 which is not in the proprietary network protocol. In yet other embodiments, the computing unit 106 of the implant 100 may be configured to operate the implant 100 or otherwise instruct the implant 100 (or an active unit 101 thereof) only using instructions received in the proprietary network protocol. In other words, the computing unit 106 may be configured to only altering an operation of the implant 100 based on data received using the proprietary network protocol.

The frequency band of the standard network protocol may in some embodiments not be included in the first frequency band of the proprietary network protocol.

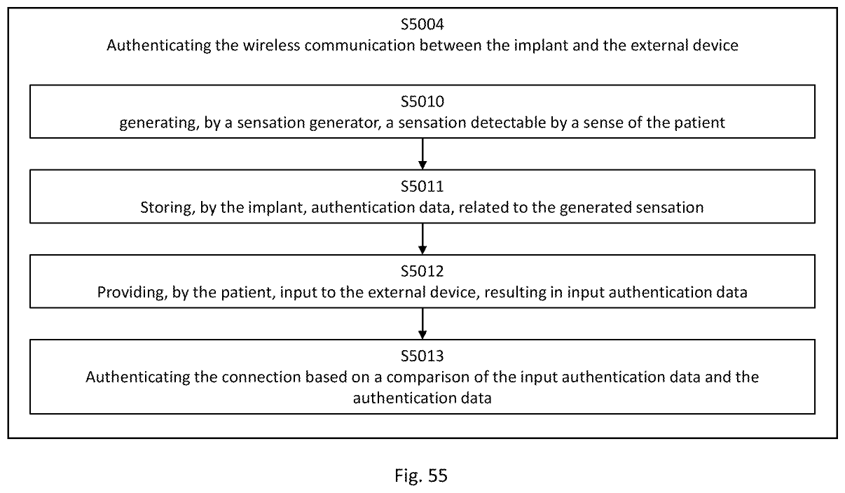

The communication between the implant 100 and the external device 200 may be further authenticated, to further increase security of communication. Such embodiments are described in FIGS. 54-55. The wireless communication between the external device 200 and the implant may be authenticated S5004. In these embodiments, following positive authentication, data between the implant and the external device (in any direction) using the proprietary network protocol may be communicated W1.

In the embodiment of FIG. 54, the authentication S5004 comprises measuring S5005 a parameter of the patient, by the external device, receiving S5006 a parameter of the patient, from the implant, comparing S5007 the parameter measured by the external device to the parameter measured by the implant, and performing S5008 authentication of a wireless connection based on the comparison. The external device may comprise sensor 250 for measuring S5005 a parameter of the patient, an external computing unit 206 configured for: receiving S5006 a parameter of the patient, from the implant, comparing S5007 the parameter measured by the external device to the parameter measured by the implant, and performing S5008 authentication of a wireless connection with the implant based on the comparison. The implant comprises an internal sensor 150 for measuring the parameter of the patient. The sensors 150, 250 may be configured to measure a pulse of the patient. The sensors 150, 250 may be configured to measure a respiration rate of the patient. The sensors 150, 250 may be configured to measure a temperature of the patient. The sensors 150, 250 may be configured to measure at least one sound of the patient. The sensors 150, 250 may be configured to measure at least one physical movement of the patient. The measured parameter, by the external device 200 may be provided with a timestamp and the measured parameter received from the implant 100 may be provided with a timestamp, wherein the comparison S5007 of the parameter measured at the implant 100 to the parameter measured by the external device 200 may comprise comparing the timestamp of the measured parameter received from the implant 100 to the timestamp of the measured parameter by the external device 200. For this reason, the external device may comprise a clock 260, configured for synchronization with a clock 160 of the implant. For example, in case the timestamps differ more than a threshold period, the wireless communication W1 is not authenticated S5008. In some embodiments, step of comparing S5007 the parameter measured by the implant 100 to the parameter measured by the external device 200 comprises calculating a difference value between the parameter measured by the implant 100 and the parameter measured by the external device 200, wherein the step of performing authentication comprises: authenticating S5008 the wireless connection W1 if the difference value is less than a predetermined threshold difference value, and not authenticating S5008 the wireless connection W1 if the difference value equals or exceeds the predetermined threshold difference value. In other embodiments, the authentication is performed by the implant 100. Further information, details and embodiments of the confirmation/authentication of the communication W1 described in FIG. 54 may be found herein under aspect 256SE. In these cases, the implant and/or external device(s) comprises the necessary features and functionality (described in the respective sections of this document) for performing such authentication.

In the embodiment of FIG. 55, the authentication S5004 comprises generating S510, by a sensation generator 181 of the implant 100, a sensation detectable by a sense of the patient, storing S5011, by the implant (in memory 107), authentication data, related to the generated sensation, providing S5012, by the patient, input to the external device 200, resulting in input authentication data, and authenticating S5013 the wireless communication W1 based on a comparison of the input authentication data and the authentication data. The authentication S5013 may be performed by either the external device 200 or the implant 100. Further information, details and embodiments of the confirmation/authentication of the communication W1 described in FIG. 55 may be found herein under the fifth and fifteenth aspects. In these cases, the implant and/or external device(s) comprises the necessary features and functionality (described in the respective sections of this document) for performing such authentication.

In some embodiments, the external device is configured to be placed in electrical connection C1 with a conductive member 201, for conductive communication with the implant. In some embodiments, such conductive communication path C1 needs to be established before the computing unit 106 of the implant alters an operation of the implant based on data received using the proprietary network protocol.

In other embodiments, such conductive communication path C1 needs to be established before the wireless receiver of the implant will received wireless communication W1 from the implant. Further information, details and embodiments of conductive communication between the implant 100 and the external device 200 using a conductive member 201 connected to the external device may be found herein under aspect 247SE. In these cases, the implant and/or external device(s) comprises the necessary features and functionality (described in the respective sections of this document) for performing such authentication.

It should also be noted that the wireless communication W1 between the external device 200 and the implant may be encrypted, for example as described herein under the second and third aspects. In these cases, the implant and/or external device(s) comprises the necessary features and functionality (described in the respective sections of this document) for performing such authentication. Consequently, even if the encrypted communication is intercepted by a third party, that third part may need to acquire ciphers or keys to decrypt the communication and access the information. This adds extra security to the communication.

Returning now to FIGS. 51-52. Further, the at least one wireless transceiver 208 is configured to communicate W2 with the second external device 300 using a standard network protocol. The external device 200 may in some embodiments be a wearable external device (such as a smart watch as in FIG. 51A, C) or a handset (such as a smart phone as in FIG. 52).

The second external device 300 may be a physical device or cloud based and may in some embodiments be operated by a caretaker of the patient, such as medical staff.

The system may in some embodiments further comprise a third external device 400, which may communicate with the second external device 300, for example using a wireless communication W4, or a wired communication. The third external device may be operated by a caretaker of the patient.

The at least one wireless transceiver 208 may comprise a first wireless transceiver 2081 configured for communicating W2 with the second external device 300, and a second wireless transceiver 2082 configured for communicating W1 with the implant 100. The external device 200 may in other embodiments comprise a computing unit 206 adapted for configuring the at least one wireless transceiver 208 to communicate W1 with the implant using the proprietary network protocol and adapted for configuring the at least one wireless transceiver 208 to communicate W2 with the second external device using the standard network protocol.

Any suitable standard protocol may be used for communication between the external device 200 and the second external device 300. Consequently, the second external device may be any device (from any manufacturer) adapted to communicate using the standard protocol. Flexibility of the system is thus improved. The standard network protocol may be one from the list of: a Radio Frequency type protocol, a RFID type protocol, a WLAN type protocol, a Bluetooth type protocol, a BLE type protocol, a NFC type protocol, a 3G/4G/5G type protocol, and a GSM type protocol.

The communication range of the proprietary network protocol may be less than a communication range of the standard network protocol. For example, the communication range may be less than 1 meter, or less than 0.5 meters. Embodiments for achieving such short-range communication is described herein under aspect 251SE. In these cases, the implant and/or external device(s) comprises the necessary features and functionality (described in the respective sections of this document) for performing such authentication. Consequently, security is further improved since the external device 200 may need to be closely positioned to the implant 100 (i.e. under control of the patient in which the implant 100 is implanted) to be able to communicate with the implant. In one embodiment, the frequency band of the proprietary network protocol is 13.56 MHz, which is the available unlicensed radio frequency ISM band for NFC/RFID type protocols. In this case, the standard network protocol may be one from the list of
WLAN type protocol;
Bluetooth type protocol
BLE type protocol
3G/4G/5G type protocol
GSM type protocol.

Advantageously, the antenna of the wireless receiver 108 of the implant 100 may be configured to only receive in this (13.56 MHz) frequency band.

In some embodiments, the wireless communication W2 between the second external device 300 and the external device 200 requires authentication to be conducted. In other words, the communication W2 between the external device 200 and the second external device 300 requires the communication to be authenticated, wherein a verification process at the second external device 300 may be used for this. Example of such verification includes authentication input at the second external device 300 being a code. In other embodiments, the authentication input at the second external device 300 is based on a biometric technique selected from the list of a fingerprint, a palm vein structure, image recognition, face recognition, iris recognition, a retinal scan, a hand geometry, and genome comparison. Consequently, the second external device 300 may comprise an interface for authentication of the communication W2 with external device 200.

As shown in FIG. 52, the system may comprise a third external device 400, and communication W4 between the second externa device 300 and a third external device 400 may be established. The communication between the second externa device 300 and the third external device may be authenticated using a verification process at the third external device 400. Example of such verification includes authentication input at the third external device 400 being a code. In other embodiments, the authentication input at the third external device 400 is based on a biometric technique selected from the list of a fingerprint, a palm vein structure, image recognition, face recognition, iris recognition, a retinal scan, a hand geometry, and genome comparison. Consequently, the third external device 400 may comprise an interface for authentication of the communication W4 with second external device 300.

Further information and definitions of features, functionality of this aspect can be found in this document in conjunction with the other aspect.

The external device may be configured to communicate further data via the conductive communication with the implant.

A computer program product of, or adapted to be run on, an external device is also provided, which comprises a computer-readable storage medium with instructions adapted to make the external device perform the actions as described above.

### Aspect 251SE 2-Part Key NFC—Two Communication Methods for Sending Encryption Keys—Embodiments of Aspect 251SE of the Disclosure

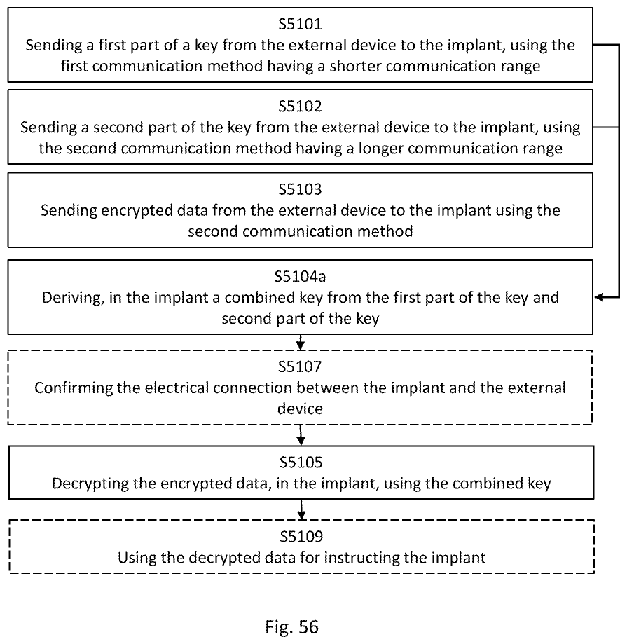

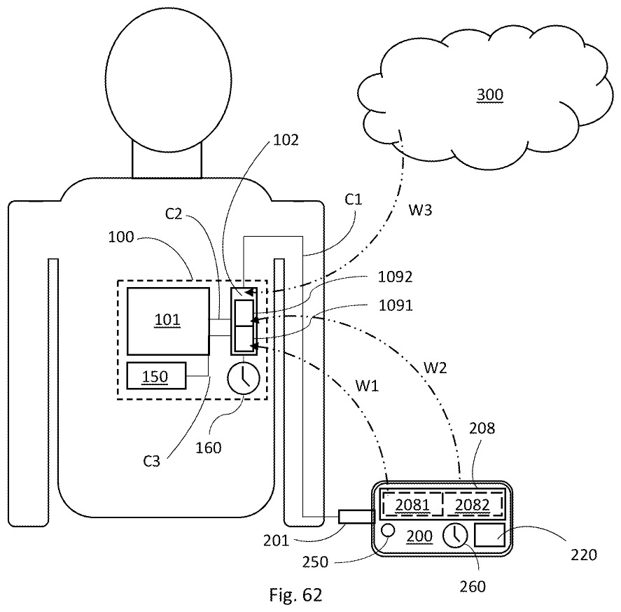

In an eight aspect, increased security for communication between an external device(s) and an implant is provided. FIGS. 56-64 shows embodiments of this aspect.

FIG. 56 shows a flow chart for methods of encrypted communication between an external device 200 and an implant 100 implanted in a patient. The external device 200 may be adapted to communicate with the implant 100 using two separate communication methods. A communication range of a first communication method W1 may be less than a communication range of a second communication method W2.

The method may comprise the steps of

Sending S5101 a first part of a key from the external device 200 to the implant 100, using the first communication method W1.

Sending S5102 a second part of the key from the external device 200 to the implant 100, using the second communication method W2.

Sending S5103 encrypted data from the external device 200 to the implant 100 using the second communication method W2.

Deriving S5104a, in the implant a combined key from the first part of the key and the second part of the key.

Decrypting S5105 the encrypted data, in the implant 100, using the combined key.

The external device 200 may be adapted to be in electrical connection C1 with the implant 100 (and vice versa), using the body as a conductor. The method may then further comprise confirming S5107 the electrical connection C1 between the implant 100 and the external device 200 and as a result of the confirmation, decrypting the encrypted data in the implant 100 and using the decrypted data for instructing the implant 100.

The method may also comprise placing a conductive member 201, configured to be in connection with the external device 200, in electrical connection with a skin of the patient for conductive communication with the implant 100. By means of the electrical connection an extra layer of security is added as a potential hacker would have to be in contact with the patient to access or affect the operation of an implant.

Using a plurality of communication methods, as described in this eighth aspect, may increase the security of the authentication and the communication with the implant as more than one channel for communication may need to be hacked or hijacked by an unauthorized entity to gain access to the implant or the communication.

The electrical connection C1 the conductive member 201 and conductive communication may be further described herein under aspect 247SE. In these cases, the implant 100 and/or external device 200 comprise the necessary features and functionality (described in the respective sections of this document).

It should also be noted that any one of the first and second communication methods W1, W2 may be needed to be confirmed in order to decrypt the encrypted data in the implant 100 and using the decrypted data for instructing the implant 100. Examples of such confirmation methods is described herein under the fifth, thirteenth and fifteenth aspect. In these cases, the implant and/or external device(s) comprises the necessary features and functionality (described in the respective sections of this document) for performing such authentication. Such example embodiments are further described below.

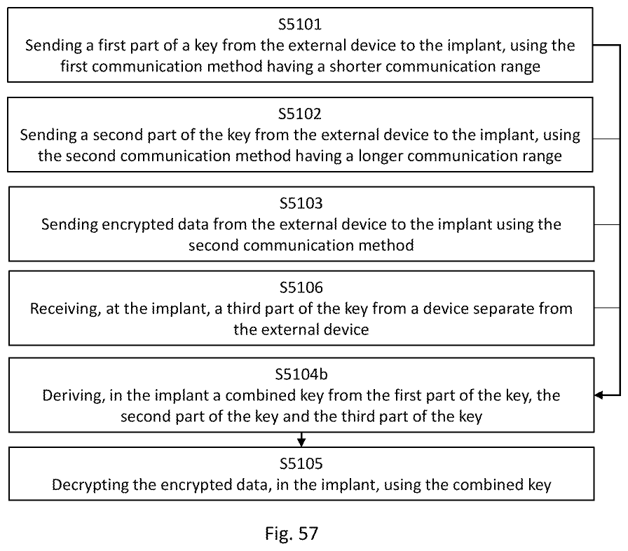

FIG. 57 shows the method further comprising the step of wirelessly receiving S5106, at the implant 100, a third part of the key from the second external device 300. In this case, the combined key may be derived S5104b from the first part of the key, the second part of the key and the third part of the key.

The first communication method W1 may be a wireless form of communication. The first communication method W1 may preferably be a form of electromagnetic or radio-based communication however, other forms of communication are not excluded. The first communication method W1 may comprise or be related to the items of the following list:
Radio-frequency identification (RFID)
Bluetooth
Bluetooth Low Energy (BLE)
Near Field Communication (NFC)
NFC-V
Infrared (IR) based communication
Ultrasound based communication

RFID communication may enable the use of a passive receiver circuit such as those in a RFID access/key or payment card. IR based communication may comprise fiber optical communication and IR diodes. IR diodes may alternatively be used directly, without a fiber, such as in television remote control devices. Ultrasound based communication may be based on the non-invasive, ultrasound imaging found in use for medical purposes such as monitoring the development of mammal fetuses.

The first communication method W1 may use a specific frequency band. The frequency band of the first communication method W1 may have a center frequency of 13.56 MHz or 27.12 MHz. These bands may be referred to as industrial, scientific, and medical (ISM) radio bands. Other ISM bands not mentioned here may also be utilized for the communication methods W1, W2. A bandwidth of the 13.56 MHz centered band may be 14 kHz and A bandwidth of the 27.12 MHz centered band may be 326 kHz.

The communication range of the first communication method W1 may be less than 10 meters, preferably less than 2 meters, more preferably less than 1 meter and most preferably less than 20 centimeters. The communication range of the first communication method W1 may be limited by adjusting a frequency and/or a phase of the communication. Different frequencies may have different rates of attenuation. By implementing a short communication range of the first communication method, security may be increased since it may be ensured or made probable that the external device is under control of the patient (holding the external device close to the implant) The communication range of the first communication method W1 should be evaluated by assuming that a patient's body, tissue, and bones present the propagation medium. Such a propagation medium may present different attenuation rates as compared to a free space of an air-filled atmosphere or a vacuum.

By restricting the communication range, it may be established that the external device communicating with the implant is in fact on, or at least proximal to, the patient. This may add extra security to the communication.

The second communication method W2 may be a wireless form of communication. The second communication method W2 may preferably be a form of electromagnetic or radio-based communication.

The second communication method W2 may be based on telecommunication methods. The second communication method W2 may comprise or be related to the items of the following list:
Wireless Local Area Network (WLAN)
Bluetooth
BLE
GSM or 2G (2nd generation cellular technology)
3G
4G
5G

The second communication method W2 may utilize the ISM bands as mentioned in the above for the first communication method W1.

A communication range of the second communication method W2 may be longer than the communication range of the first communication method W1. The communication range of the second communication method W2 may preferably be longer than 10 meters, more preferably longer than 50 meters, and most preferably longer than 100 meters.

Encrypted data may comprise instructions for updating a control program 110 running in the implant 100. Encrypted data may further comprise instructions for operating the implant 100.

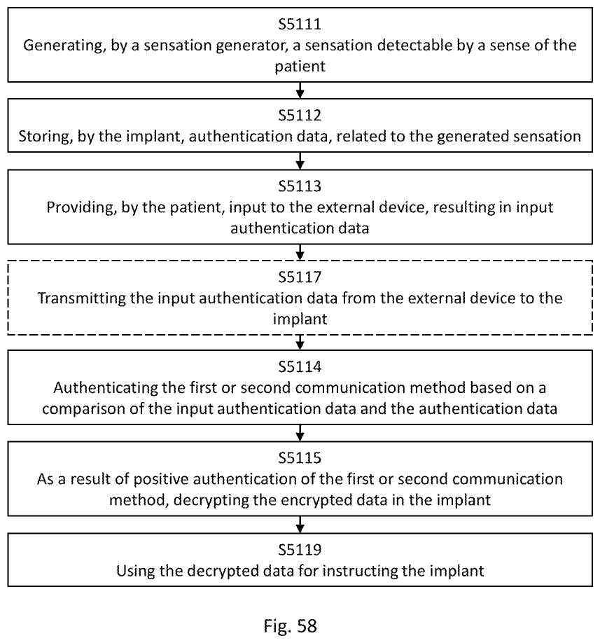

FIG. 58 shows a flow chart of further steps which may be appended to the method for authenticating the communication methods W1, W2 by generating sensations detectable by the patient in which the implant 100 is implanted. For this the method may further comprise:

Generating S5111, by a sensation generator 181, a sensation detectable by a sense of the patient.

Storing S5112, by the implant 100, authentication data, related to the generated sensation.

Providing S5113, by the patient, input to the external device 200, resulting in input authentication data.

Authenticating S5114 the first or second communication W1, W2 method based on a comparison of the input authentication data and the authentication data.

As a result of positive authentication of the first or second communication method W1, W2, decrypting S5115 the encrypted data in the implant 100.

Using S5119 the decrypted data for instructing the implant.

The method may further comprise the step of transmitting S5117 the input authentication data from the external device 200 to the implant 100. In this case the comparison of the input authentication data and the authentication data may be performed by or at the implant 100.

The sensation generator 181, sensation, authentication data, input authentication data, as well as further methods for authentication based on sensations may be further described herein under the fifth and fifteenth aspects. In these cases, the implant 100 and/or external device(s) comprises the necessary features and functionality (described in the respective sections of this document). Using sensations for authenticating the communication or communication method provides an extra level of security as sensations may be adapted to only be sensible by the patient, thus preventing unauthorized access or authentication.

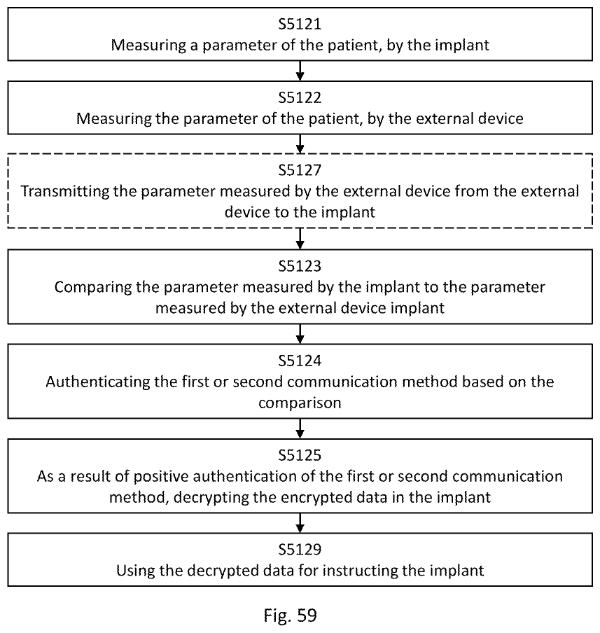

FIG. 59 shows a flow chart of further steps which may be appended to the method for authenticating the communication methods W1, W2 by measuring a parameter of the patient by the implant 100 and the external device 200. For this the method may further comprise:

Measuring S5121 a parameter of the patient, by the implant 100.

Measuring S5122 the parameter of the patient, by the external device 200.

Comparing S5123 the parameter measured by the implant 100 to the parameter measured by the external device 200.

Authenticating S5124 the first or second communication method W1, W2 based on the comparison.

As a result of positive authentication of the first or second communication method W1, W2, decrypting S5125 the encrypted data in the implant 100.

Using S5129 the decrypted data for instructing the implant 100.

The method may further comprise transmitting S5127 the parameter measured by the external device 200 from the external device 200 to the implant 100. In this case, the comparison of the parameter of the patient measured by the external device 200 and the parameter of the patient measured by the implant 100 may be performed by the implant 100. The implant 100 may comprise a first sensor 150 for measuring the parameter of the patient at the implant 100. The external device 200 may comprise an external sensor 250 for measuring the parameter of the patient at the external device 200.

The parameters of the patient, the first and second sensors 150, 250, as well as further methods for authentication based on measuring parameters of the patient may be further described herein under aspect 256SE. In these cases, the implant 100 and/or external device(s) comprises the necessary features and functionality (described in the respective sections of this document). The use of parameters of the patient may provide extra security for the communication or communication method.

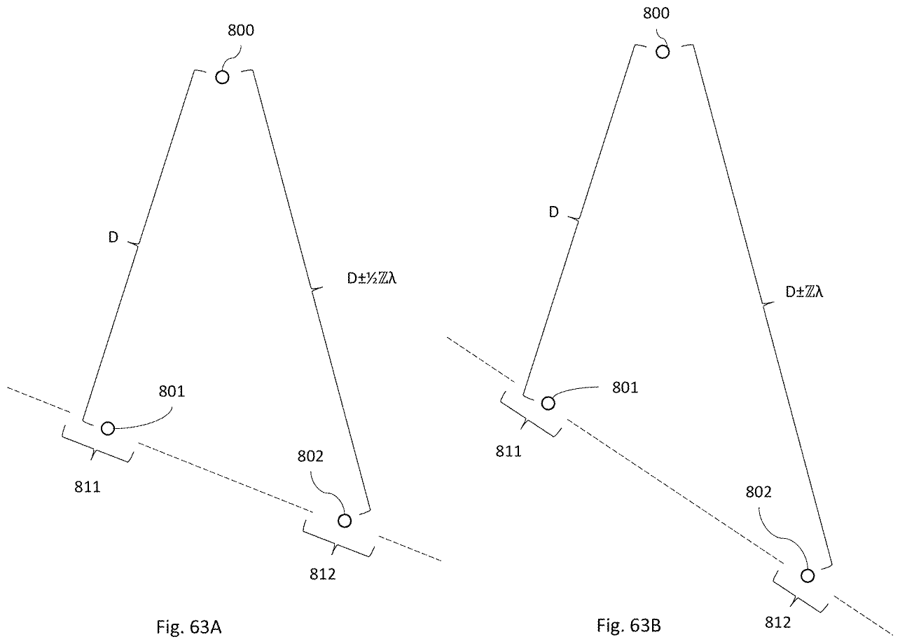

The communication or communication methods W1, W2 between the implant 100 and the external device 200 may be cancelled or amplified for at least one point 800 by destructive or constructive interference/diffraction. As illustrated by FIGS. 63A-B, this may be achieved by transmitting the communication with a wavelength, λ, from a first point 801 located a distance, D, away from the at least one point 800, and by also transmitting the communication from a second point 802, located at either a distance D±½Zλ or D±Zλ from the at least one point 800. Herein, Z may be any integer, e.g. −4, −3, −2, −1, 0, 1, 2, 3, 4 etc. The communication may be cancelled for the at least one point 800 by transmitting from the second point 802, located at a distance D±½Zλ from the at least one point 800, as shown in FIG. 63A. The communication may be amplified for the at least on point 800 by transmitting from the second point 802, located at a distance D±Zλ from the at least one point 800, as shown in FIG. 63B.

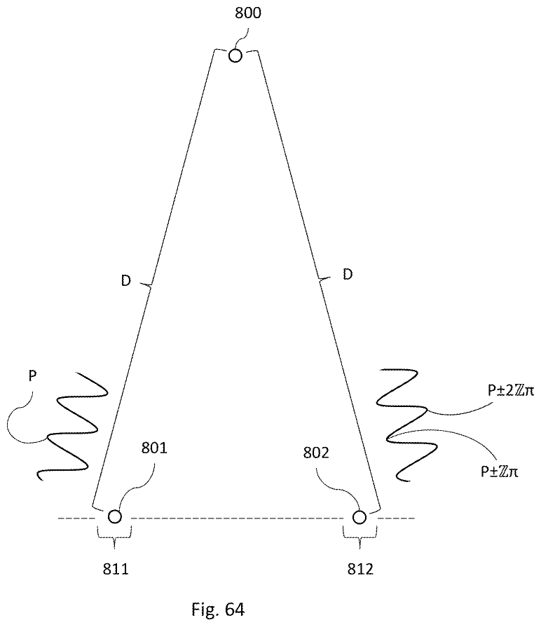

FIG. 64 illustrates another way for cancelling or amplifying the communication. The communication may in this example be transmitted from a first point 801 with a phase, P. The communication may be cancelled for the at least one point 800 by transmitting the communication from a second point 802 with a phase P±Zπ. The communication may instead be amplified for the at least one point 800 by transmitting the communication from the second point 802 with a phase P±2Zπ. Once more, Z may be any integer, e.g. −4, −3, −2, −1, 0, 1, 2, 3, 4 etc.

A distance between the first point 801 and the at least one point 800 may equal the distance between the second point 802 and the at least one point 800 plus or minus any integer times a wavelength, λ, of the communication.

Alternatively, combinations of using different phases for the communication transmitted from the first and second points 801, 802 and using different distances between the first and second points 801, 802 and the at least one point 800 may be used to cancel or amplify the communication.

The first point 801 may be a first transmitter and the second point 802 may be a second transmitter. The first point 801 and the second 802 point may be moved with respect to each other such that the at least one point 800 is spatially shifted. Preferably, the first point 801 and the second point 802 are associated with the external device 200 and the at least one point 800 is associated with the implant 100.

The at least one point 800 may be one of a plurality of points where the communication is cancelled or amplified. The first point 801 may be associated with the implant 100 and the second point 802 may be associated with the external device 200 (or vice versa). The first point 801 may be a first slit 811 and the second point 802 may be a second slit 812. The first and second slits 811, 812 may be adapted to receive the same communication from a single transmitter. The transmitter may be located equidistant to the first and second slits 811, 812.

A phase, P, of the communication may be alternated as to spatially shift the at least one point.

This may be done to provide, a moveable, localized point where even very low amplitude signals may interfere and have a high amplitude such that the communication may be improved. This may aid in calibrating an implant and external device system towards individual and different patient bodies.

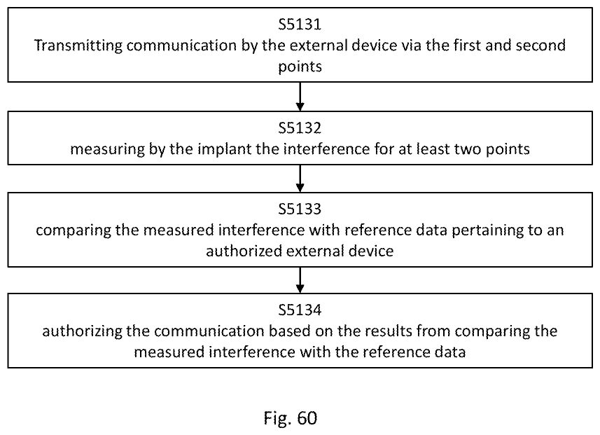

FIG. 60 shows a flow chart of a method for authenticating the communication based on patterns of constructive and/or destructive interference. The method may then comprise: Transmitting S5132 by the external device 200 via the first and second points 801, 802.

Measuring S5132 by the implant 100 the interference for at least two points.

Comparing S5133 the measured interference with reference data pertaining to an authorized external device 200.

Authenticating S5134 the communication based on the results from comparing the measured interference with the reference data.

Such a method may provide increased security by verifying that the external device is at specific positions, or at least specific directions, relative to the implant. This is made possible by comparing expected interference patterns with measurements of signal strength at actual points (first and second) by the implant. A precise distance may also be evaluated based on the interference pattern and thus further narrow the tolerance for the spatial positioning of the external device 200. A plurality of points, larger than two, may be measured and compared against for authenticating the communication. Reference data pertaining to an authorized external device 200 may comprise an interference pattern or data related to interference observed or expected from a trusted or authorized external device 200. The reference data may be calibrated to match a trusted or authorized external device 200.

An interference pattern may be dependent on the following factors:
Type of transmission (i.e. electromagnetic, sound, etc.)
Number of transmitters
Location of transmitters
Directivity of transmitters
Power of transmission
Wavelength of transmission
Phase of transmission
Medium
Reflections

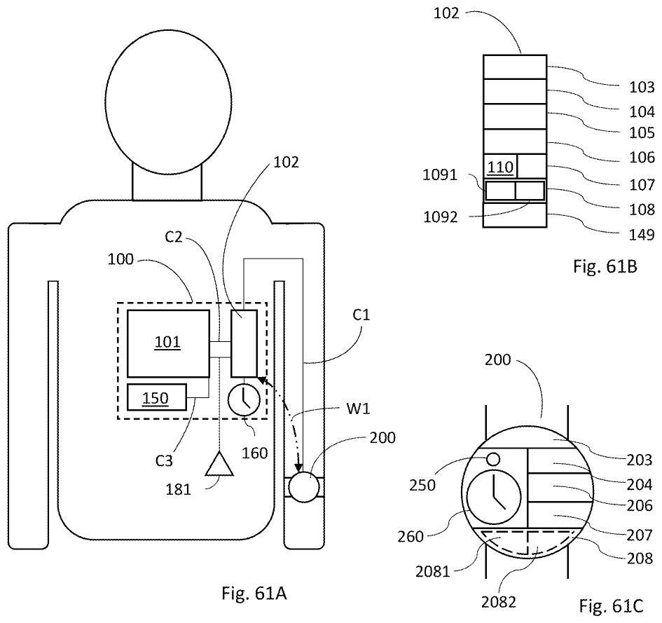

FIGS. 61A-C and 62 show an implant 100 and an external device 200 as well as a system comprising both and an optional second external device 300. The implant 100, the external device 200, and the system may be configured for performing the methods and actions discussed herein.

The external device 200 may comprise a wireless transceiver 208. The wireless transceiver 208 may in turn comprise a first wireless transceiver 2081 and a second wireless transceiver 2082. The first wireless transceiver 2081 may be configured for communication with the implant 10 using the first communication method W1. The second wireless transceiver 2082 may be configured for communication with the implant 10 using the second communication method W2.

The first wireless transceiver 2081 may be configured to send a first part of a key to the implant 100, using the first communication method W1. The second wireless transceiver 2082 may be configured to send a second part of a key to the implant 100, using the second communication method W2. The second wireless transceiver 2082 may further be configured to send encrypted data to the implant using the second communication method W2. The first communication method W1 may be used by the first wireless transceiver 2081. The second communication method W2 may be used by the second wireless transceiver 2082.

The first wireless transceiver 2081 may be configured to limit the communication range of the first communication method W1 by adjusting the frequency and/or phase of the transmitted information.

The external device 200 may comprise a loop antenna. The loop antenna may be part of the wireless transceiver 208 or the first or second wireless transceiver 2081, 2082.

The external device 200, or the wireless transceiver 208 may be used to transmit the communication from the first and second points 801, 802. The first point 801 may correspond to the first wireless transceiver 2081 and the second point 802 may correspond to the second wireless transceiver 2082. The at least one point 800 may correspond to a wireless transceiver 108 of the implant 100. By configuring the points and the phases of transmission such that the communication is amplified at the at least on point 800, the communication transmission strength/power at the first and second points 801, 802 may be reduced.

The implant 100 may comprise a wireless receiver. The implant 100 may comprise a first wireless receiver 1091. The implant 100 may comprise a second wireless receiver 1092. The wireless receivers 1091, 1092 may be part of the wireless transceiver 108. The first communication method W1 may be received by the first wireless receiver 1091. The second communication method W2 may be received by the second wireless receiver 1092.

The implant 100 may comprise a passive receiver for receiving the first part of the key. The passive receiver of the implant 100 may comprise a loop antenna. The first and second wireless receivers 1091, 1092 may comprise the passive receiver. The passive receiver may be a receiver that does not require a power source but rather uses the energy of the radio waves it receives to power it. Generally, such a passive receiver would need the received communication to be transmitted with higher power or by a transmitter located closer to the receiver. The passive receiver may be adapted to only receive transmitted communication. Such a receiver may conserve energy of the implant as the passive receiver may be powered by the received radio waves.

The implant 100 may comprise a computing unit 106. The computing unit 106 may be configured to update the control program 110 running in the implant 100 using the decrypted data. The computing unit 106 may be configured to operate the implant 100 using the decrypted data. The computing unit 106 may be configured to derive a combined key from the first part of the key and the second part of the key, and decrypt the encrypted data using the combined key.

The computing unit 106 may further be configured for: Receiving a parameter of the patient, from the external device 200.

Comparing the parameter measured by the implant 100 to the parameter measured by the external device 200.

Authenticating the first or second communication method W1, W2 based on the comparison.

As a result of positive authentication of the first or second communication method W1, W2, decrypting the encrypted data in the implant 100 and using the decrypted data for instructing the implant 100.

The implant 100 may further be configured to wirelessly receive a third part of the key from the second external device 300. The computing unit 106 may then be configured to derive the combined key from the first part of the key, the second part of the key and the third part of the key.

The implant 100 may comprise an authentication unit configured to confirm an electrical connection C1 between the implant 100 and the external device 200. The computing unit 106 may then be configured for, as a result of the confirmation, decrypting the encrypted data and using the decrypted data for instructing the implant.

The implant 100 may be connected to or comprise the sensation generator 181 and be configured for:

Storing authentication data, related to a sensation generated by the sensation generator 181.

Receiving input authentication data from the external device 200.

The internal computing unit 106 may then be configured for:

Authenticating the first or second communication method W1, W2 based on the comparison.

As a result of positive authentication of the first or second communication method W1, W2, decrypting the encrypted data in the implant 100 and using the decrypted data for instructing the implant 100.

Authentication data and measured parameters of the patient may be stored by a memory 107 of the implant 100. The control program 110 may also be stored by the memory 107. The memory 107 may be a digital storage medium, adapted for storing digital information or data.

The implant 100 may be configured for:

Receiving the communication from the first and second points 801, 802 of the external device 200.

Measuring the interference for at least two points.

Comparing the measured interference with reference data pertaining to an authorized external device 200.

Authenticating the communication based on the results from comparing the measured interference with the reference data.

The system comprising the implant 100, external device 200 and the optional second external device 300 may comprise the conductive member 201 configured to be in electrical connection with the external device 200. The conductive member 201 may be configured to be placed in electrical connection with a skin of the patient for conductive communication with the implant 100 using the electrical/conductive connection C1.

The second external device 300 may be configured for communication with the external device 200. The external device 200 may be configured for receiving the encrypted data from the second external device 300 and relaying the encrypted data to the implant 100 using the first and/or the second communication method W1, W2.

The communication between the second external device 300 and the external device 200 may use a third communication method W3. The third communication method W3 may be a wireless communication method as those proposed for the first and second communication methods W1, W2. The third communication method W3 may alternatively be a wired/electrical/conductive communication method.

The second external device 300 may comprise an interface for authentication of the communication with the external device 200. Communication between the external device 200 and the second external device 300 may require the communication to be authenticated. Authentication may be performed by the various methods described herein under this and the other aspects.

The relaying of encrypted data between the second external device 300 and the external device 200 is further described herein under aspect 253SE. In these cases, the implant 100 and/or external device 200 comprise the necessary features and functionality (described in the respective sections of this document). Using the external device 200 as a relay, with or without verification from the patient, may provide an extra layer of security as the external device 200 may not need to store or otherwise handle decrypted information. As such, the external device 200 may be lost without losing decrypted information. The combination using two communication methods with a relaying device may be advantageous as the incoming and outgoing transmissions may interfere less with each other.

The second external device 300 may be operated by a healthcare provider of the patient. A healthcare provider may be a healthcare professional such as a physician or a nurse.

The term communication may in some cases refer to the first communication method at least within this aspect. The term may in some cases refer to the second communication method at least within this aspect. The term may in some cases refer to the third communication method at least within this aspect.

NFC-V may be understood as relating to a longer-range NFC class. The maximum communication range of NFC-V may be understood as being in the range from 1-2 meters.

The method may further comprise confirming, by the patient, the communication between the external device and the implant.

The method may further comprise sending a third part of the key from the external device to the implant, using a conductive communication method, wherein the combined key is derived from the first part of the key, the second part of the key and the third part of the key.

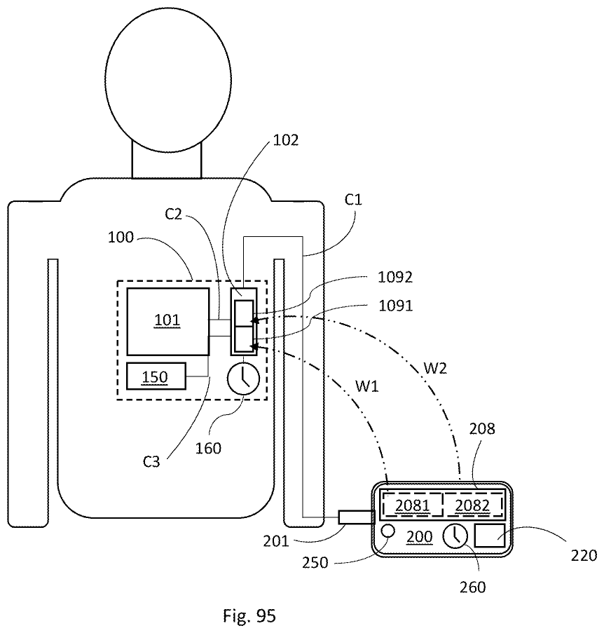

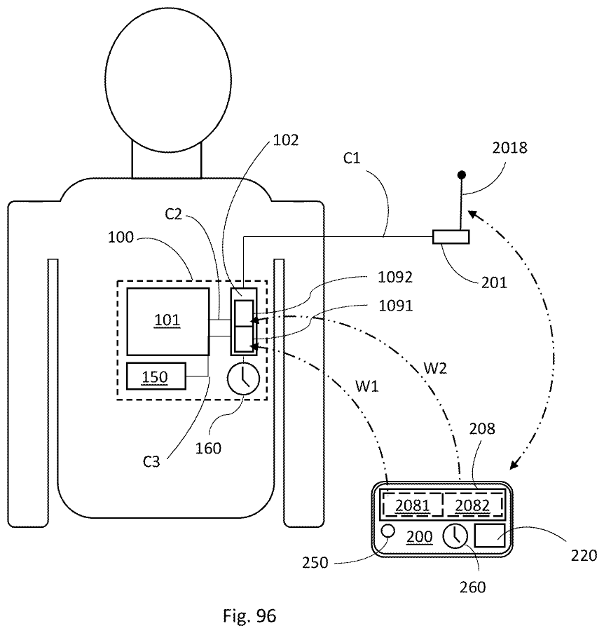

A system comprising an external device 200 is shown in FIG. 95 and FIG. 96. The system further comprises a conductive member 201 configured to be placed in electrical connection with a skin of a patient for conductive communication C1 with an implant 100 implanted in the patient. The conductive member 201 may be integrally connected to the external device 200. The conductive member 201 may comprise a wireless communication interface 2018 and is communicatively connected to the external device 200. The wireless communication interface 2018 may be at least one antenna element.

The implant may comprise at least one of
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

### Aspect 252SE Dual Systems—Two Communication Systems for Communication Between Implant and External Device—Embodiments of Aspect 252SE of the Disclosure

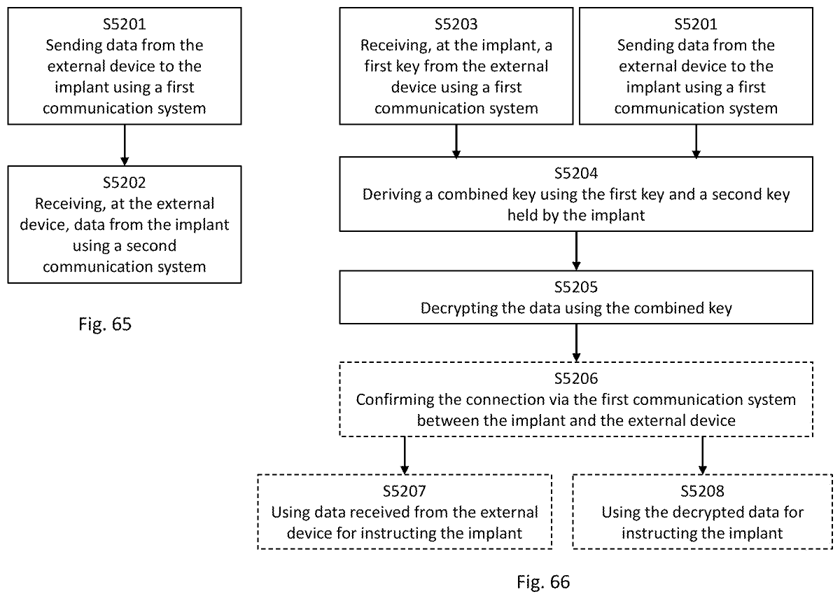

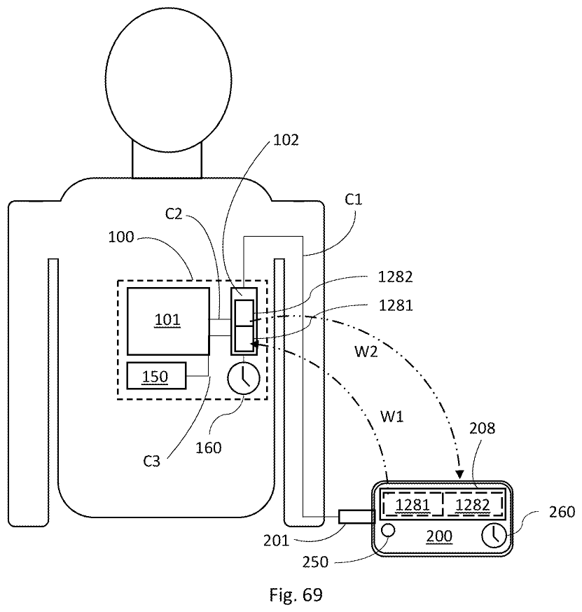

In aspect 252SE, increased security for communication between external device(s) and an implant is provided. FIGS. 65-70 shows embodiments of this aspect.

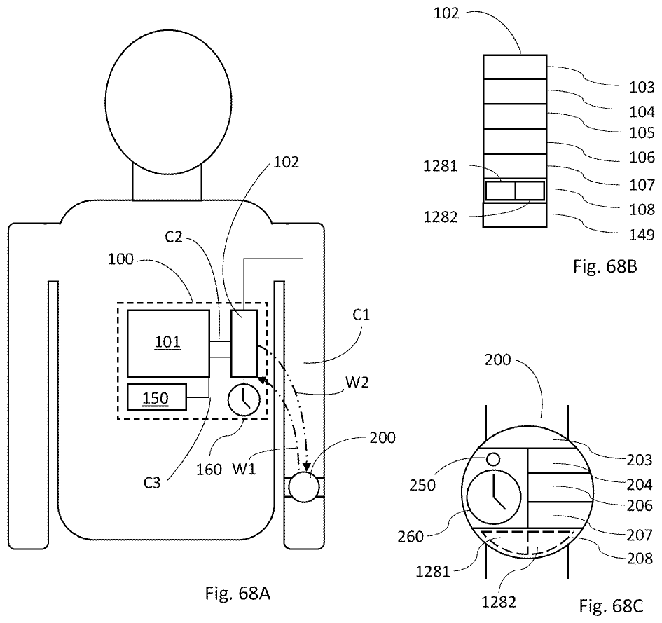

FIG. 1 shows a first embodiment of aspect 252SE, which will be described in conjunction with FIGS. 68A-C and 69. In this embodiment, communication between an external device 200 and an implant 100 when implanted in a patient is provided. This is achieved by a first communication system (transmitting wireless communication W1) for sending S5201 data, that may or may not be encrypted, from the external device 200 to the implant 100, and using a second, different, communication system (transmitting wireless communication W2) for receiving S5202, at the external device 200, data from the implant 100. By using different communication systems, a more flexible approach to transmission of data between the implant 100 and the external device 200 is provided. For example, different levels of security for transmissions to and from the implant 100 may be implemented. For example, the implant 100 may be configured to only receive data to be used for instructing the implant 100 from the external device 200. This may be achieved by using a proprietary network protocol for communication using the first communication system. In other embodiments, the communication of data from the implant 100 to the external device 200 is sensitive such that a proprietary network protocol for communication using the second communication system is implemented. Consequently, the first communication system may be configured for wireless communication W1 using a first network protocol, and the second communication system may be configured for wireless communication W2 using a second, different, network protocol. In some embodiments, the first or second network protocol is a proprietary network protocol, wherein the other network protocol is a standard network protocol.

Further details relating to different network protocols is described herein under aspect 250SE. In applicable cases, the implant and/or external device(s) comprises the necessary features and functionality (described in the respective sections of this document) for achieving transmissions with different network protocols.

In other embodiments, different communication ranges of the first and second communication systems are implemented. For example, the communication range of the first communication system may be less than the communication range of the second communication system or vice versa. For example, the communication range of the first or second communication system may be less than 1 meter, or less than 0.5 meters. Embodiments for achieving such short-range communication is described herein under aspect 251SE. In these cases, the implant and/or external device(s) comprises the necessary features and functionality (described in the respective sections of this document) for achieving such short-range communication. In these cases, the two communication systems may correspond to the two communication methods or communication protocols. Having the communications physically separated on different systems further increase security, compared to using the same physical system for all communications, presenting a single point of failure.

Consequently, security is further improved since the external device 200 may need to be closely positioned to the implant 100 (i.e. under control of the patient in which the implant 100 is implanted) to be able to transmit data to the implant 100 or receive data from the implant 100.

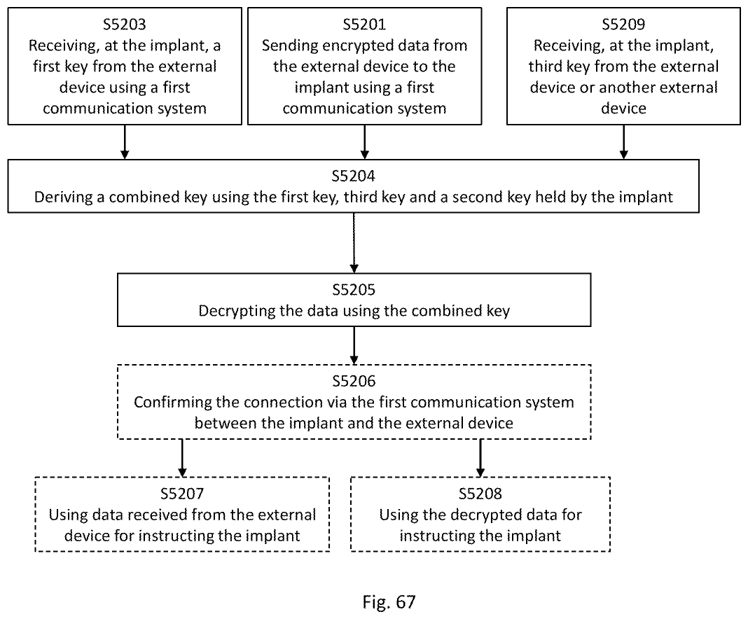

The data received S5201 at the implant may be decrypted according to embodiments described in FIGS. 66-67.

In one embodiment, the implant comprises a computing unit 106 configured for receiving S5203, at the implant, a first key from an external device. The key may be received using the first communication system by wireless communication W1. In other embodiments, conductive communication C1 may be used for transmitting the first key. Conductive communication may be achieved by the use of a conductive member 201 configured to be in electrical connection with the external device 200, the conductive member 201 being configured to be placed in electrical connection with a skin of the patient for conductive communication C1 with the implant 100. The feature of conductive communication C1 may be achieved as described herein under aspect 247SE. In these cases, the implant and/or external device(s) comprises the necessary features and functionality (described in the respective sections of this document) for performing such conductive communication. The communication may thus be provided with an extra layer of security in addition to the encryption by being electrically confined to the conducting path e.g. external device 200, conductive member 201, conductive connection C1, implant 100, meaning the communication will be excessively difficult to be intercepted by a third party not in physical contact with, or at least proximal to, the patient.

The computing unit 106 is further configured for deriving S5204 a combined key using the first key and a second key held by the implant (e.g. in memory 107), decrypting S5205 the data using the combined key, and using the decrypted data for instructing S5208 the implant.

In some embodiments, to further increase security, the connection W1 between the implant 100 and the external device 200 (i.e. the first communication system) needs to be authenticated/confirmed before instructing S5207 the implant using the decrypted data. Consequently, in some embodiments, the computing unit 106 is configured for confirming the connection via the first communication system between the implant and the external device. As a result of the confirmation (i.e. upon positive confirmation/authentication) the computing unit may instruct S5208 the implant based on the decrypted data. The decrypted data may comprise at least one of data for updating a control program running in the implant, and operation instructions for operating the implant.

The confirmation and authentication of the wireless communication W1 may be performed as described herein under the fifth, thirteenth and fifteenth aspect. In these cases, the implant and/or external device(s) comprises the necessary features and functionality (described in the respective sections of this document) for performing such authentication. By communication according to these aspects, security of the communication may be increased as it may require a malicious third party to know or gain access to either the transient physiological parameter of the patient or detect randomized sensations generated at or within the patient.

In other embodiments, the external device 200 is configured to confirm the connection W1, via the first communication system, between the implant and the external device, and the external device 200 is configured to communicate further data to the implant following positive confirmation, which then may be used for instructing S5207 the implant. Such further data may be transmitted using the first communication system and may or may not be encrypted as described herein. The further data may comprise at least one of: data for updating a control program running in the implant, and operation instructions for operating the implant

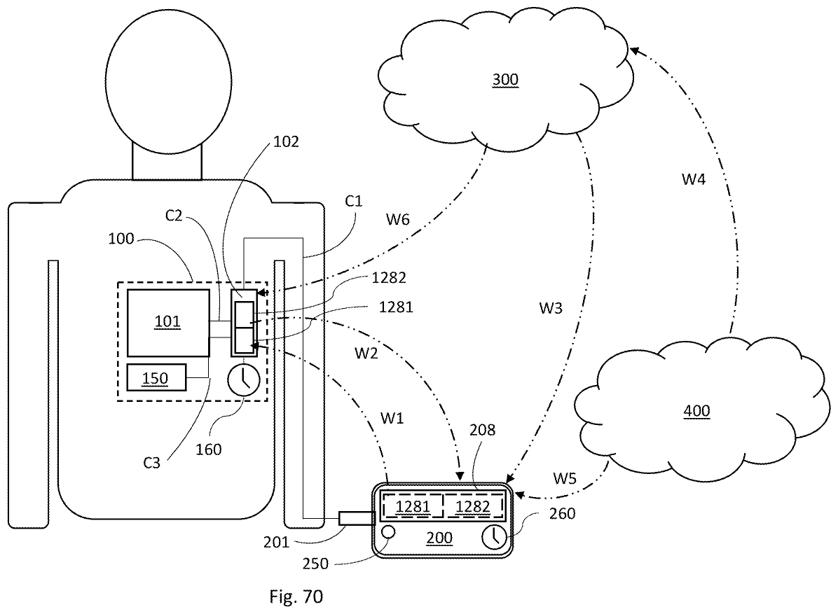

FIG. 67 and FIG. 70 shows other embodiments for increasing security of communication between the implant 100 and the external device 200. In these embodiments, a third key is used for encryption/decryption of the data sent S5201 from the external device 200 to the implant 100. The third key is generated by a second external device 300, separate from the external device or by a another external device 400 being a generator of the second key on behalf of the second external device. The second external device 300 may be under control of a caretaker of the patient, such as medical staff. The another external device 400 may e.g. be a device under control of the IT department of a hospital which is adapted to compute encryption keys on behalf of (upon being instructed by) the second external device.

The third key is received S5209 at the implant from anyone of the external device 200 (e.g. using the wireless communication W2 of the first communication system, or using conductive communication C1), the second external device (using the wireless communication W6), and the generator of the second key (using wireless communication not included in FIG. 70). In some embodiments, there may exist a third communication system (wireless communication W6), the third communication system being different than the first and second communication system, for sending data (e.g. the third key) from the second external device 300, separate from the external device 200, to the implant 100.

In case the external device 200 is transmitting the third key, the external device 200 may receive the third key from the second external device 300 using wireless transmission W3, or wired communication (i.e. ethernet, LAN, not included in the drawings). The external device 200 may receive the third key from another external device 400 using wireless transmission W5, or wired communication (i.e. ethernet, LAN, not included in the drawings). The routing/relaying functionality of the third key at the external device 200 may be performed as described herein under aspect 253SE. In these cases, the implant and/or external device(s) comprises the necessary features and functionality (described in the respective sections of this document) for performing such routing/relaying functionality. Using the external device 200 as a relay, with or without verification from the patient, may provide an extra layer of security as the external device 200 may not need to store or otherwise handle decrypted information. As such, the external device 200 may be lost without losing decrypted information. The combination using two communication systems with a relaying device may be advantageous as the incoming and outgoing transmissions may interfere less with each other.

The second external device 300 may be connected to said another external device 400 and receive data using wireless transmission W4, or wired communication (i.e. ethernet, LAN, not included in the drawings).

When the third key is used, the computing unit 106 may be configured to derive S5204 the combined key using the first and third keys and the second key held by the implant to decrypt the data. The decrypted data may then be used for instructing S5208 the implant. Also, in this embodiment, the computing unit 106 may be configured to first authenticate/confirm S5206 the wireless communication W1 prior to using the decrypted data for instructing the implant 100.

The data received S5202 at the external device from the implant 100 using the second communication system (i.e. wireless communication W2) may comprise feedback signals from the implant including one or more from the list of: physiological or physical sensor parameters related to the status of the body of the patient, and physical or functional parameters related to status of the implant. Further examples on what feedback the implant may transmit (based on functionality the implant) is described herein under aspect 255SE in which cases, the implant and/or external device(s) comprises the necessary features and functionality (described in the respective sections of this document).

The two communication systems may be achieved by one or more wireless transceivers (or separate receiver(s), transmitter(s)) 108 in a communication unit 102 of the implant, which communication unit 102 is connected to an active unit 101 of the implant. The one or more wireless transceivers 108 of the implant 100 are adapted for communication with one or more wireless transceivers 208 (or separate receiver(s), transmitter(s)) of the external device 200. The first communication system is schematically referred to as 1281 in the drawings. The second communication system is schematically referred to as 1282 in the drawings

The two communication systems 1281, 1282 may for example be implemented according to the following.

The first communication system 1281 may be implemented using a first wireless receiver at the implant 100, or a first wireless transceiver at the implant 100. The first wireless receiver/transceiver may be configured for receiving data from a first wireless transmitter/transceiver in the external device 200.

The second communication system 1282 may be implemented using a first wireless transmitter at the implant 100, or a second wireless transceiver at the implant 100. The first wireless transmitter, or second wireless transceiver may be configured for transmitting data to a first wireless receiver, or a second wireless transceiver in the external device 200. In these embodiments, the first communication system 1281 is implemented using a first antenna of the implant and a first antenna of the external device, and the second communication system 1282 is implemented using a second antenna of the implant and a second antenna of the external device

In some embodiments, the first and second communication systems 1281, 1282 may be implemented using a single wireless transceiver in the implant and a single wireless transceiver in the external device (i.e. one antenna at the implant and one antenna at the external device), but where for example the network protocol used for data transmission from the external device 200 to the implant 100 is different from the network protocol used for data transmission from the implant 100 to the external device 200, thus achieving two separate communication systems 1281, 1282.

Further information and definitions of features and functionality of this aspect can be found in this document in conjunction with the other aspects.

The data sent from the external device to the implant may be encrypted data. The data sent to the implant may be encrypted data. The data received from the external device may be encrypted data.

According to embodiments of the third part of aspect 252SE, the first communication system may be a conductive communication system, configured for conductive communication. The feature of conductive communication C1 may be achieved as described herein under aspect 247SE. In these cases, the implant and/or external device(s) comprises the necessary features and functionality (described in the respective sections of this document) for performing such conductive communication.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

### Aspect 253SE Passive Proxy—Passive Proxy—Embodiments of Aspect 253SE of the Disclosure

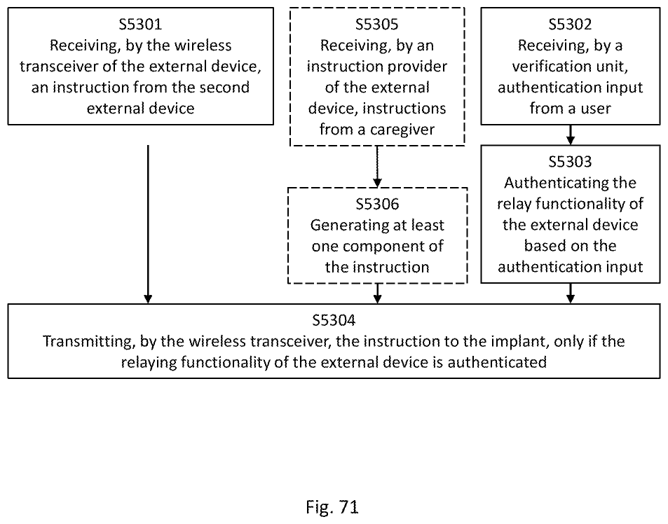

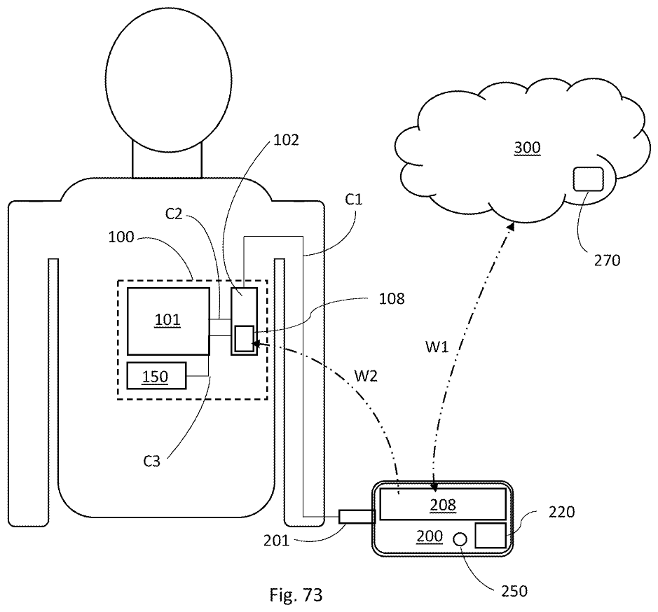

In aspect 253SE, increased security for communication between external device(s) and an implant is provided. FIGS. 71-73 shows embodiments of this aspect.

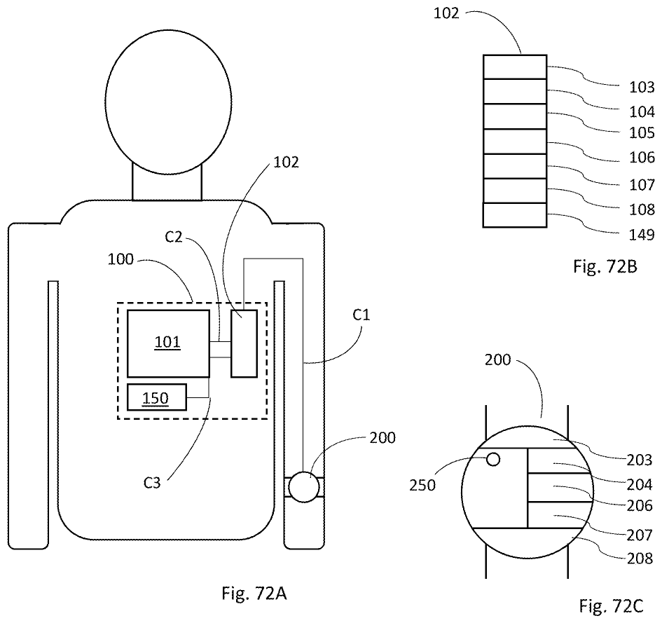

FIGS. 72A-C and 73 show a system comprising an external device 200, an implant 100, implanted in a patient, and a second external device 300, other than the external device 200.

The external device 200 may comprise a wireless transceiver 208. The wireless transceiver may be configured for wireless communication with the second external device 300 and the implant 100. The wireless communication may use a wireless connection W1 between the second external device 300 and the external device 200. The wireless communication may use a wireless connection W2 between the external device 200 and the implant 100.

The wireless transceiver 208 may be configured to receive an instruction from the second external device 300. The instruction may or may not be communicated using a first network protocol. The wireless transceiver 208 may further be configured to transmit the instruction to the implant 100. The instruction may or may not be transmitted using a second network protocol.

The wireless transceiver 208 may comprise more than one transceiver. Separate transceivers may be utilized for the first and the second network protocols. The external device 200 may alternatively comprise separate transmitters and receivers rather than having them integrated or comprised in a transceiver.

The first network protocol may be a standard or open network protocol. Types of standard network protocols include:
Radio-frequency type protocol
RFID type protocol
WLAN
Bluetooth
BLE
NFC
3G/4G/5G
GSM

Generally, the first network protocol may be any type of standard wireless communication method such as wireless telecommunication methods or radio communication methods.

The second network protocol may be a proprietary network protocol. The second network protocol may be based on non-public, non-standard, or limited access wireless communication methods with increased security.

Further examples of communication methods and communication protocols that may be utilized for transmitting the instructions, in conjunction with or instead of using, the first and second network protocols as well as the wireless transceiver 208 may be described herein under the eighth and ninth aspects. In these cases, the implant 100 and/or external device 200 comprise the necessary features and functionality (described in the respective sections of this document). The combination using two communication methods or systems with a relaying device may be advantageous as the incoming and outgoing transmissions may interfere less with each other.

Alternatively, to using the wireless connection W2 for transmitting the instructions from the external device 200 to the implant 100, an electrical/conductive connection C1 may be used. For this, the system may further comprise a conductive member 201, configured to be in electrical connection with the external device 200. The conductive member 201 may be configured to be placed, or placed, in electrical connection with a skin of the patient for conductive communication with the implant 100. The external device 200 may be configured to be placed in electrical connection with the conductive member 201, for conductive communication with the implant 100. Conductive communication using the electrical/conductive connection C1 may be used to communicate the instructions between the external device 200 and the implant 100. Conductive communication using the electrical/conductive connection C1 may also be used for authenticating the external device 200 and its authenticity as an authorized relaying device of the instructions from the second external device 300 to the implant 100 via the external device 200.

The electrical/conductive connection C1 the conductive member 201 and conductive communication may be further described herein under aspect 247SE. In these cases, the implant 100 and/or external device 200 comprise the necessary features and functionality (described in the respective sections of this document).

The instruction may comprise instructions for operating the implant 100 or instructions for updating a control program of the implant 100. The instruction received at the external device 200 may be encrypted. The external device 200 may be configured to transmit the instruction to the implant 100 without decrypting the instruction.

The instruction may be provided to the second external device 300 by a trusted source of origin, such as a manufacturer/supplier of the implant 100 or a caregiver of the patient, in which the implant is implanted. The caregiver may be a health care provider of the patient. The caregiver may be a healthcare professional such as a physician or a nurse. The second external device 300 may be controlled, operated or in the possession of the caregiver.

The external device 200 may also comprise a verification unit 220. The verification unit 220 may be configured to receive authentication input form a user for authenticating a relaying functionality of the external device 200. The wireless transceiver 208 may further be configured to: upon authentication of the relaying functionality of the external device 200, cause the wireless transceiver 208 to transmit the instruction to the implant 100; and upon non-authentication or failed authentication of the relaying functionality of the external device 200, cause the external device 200 to hold the instructions. In this context, to hold means to keeping the instructions in place i.e. not transmitting them to the implant 100.

Such an external device, as described in this tenth aspect, may be made simple with a small footprint and yet enable secure communication as user authentication/verification is required for the external device to relay communication to the implant.

The relaying functionality of the external device 200 refers to its role as a relaying device of the instructions from the second external device 300 to the implant 100 via the external device 200.

The user may be the patient in which the implant 100 is implanted. The user may alternatively be the caregiver.

The authentication input may comprise a parameter of the patient, in which case the authentication input may be provided by the patient. Authentication input may also comprise a parameter of the caregiver, in which case the authentication input may be provided by the caregiver. The authentication input from the user may comprise a code. The code may be provided by either a patient or a caregiver. The authentication input may comprise a single use code.

The external device 200 may comprise an instruction provider 270. Alternatively, the second external device 300 may be considered to be or comprise the instruction provider 270. The instruction provider may be adapted to receive instructions from a caregiver generating at least one component of the instruction. The external device 200 or the instruction provider 270 may be adapted to receive authentication input from the caregiver, comprising at least one of a code and a parameter of the caregiver.

A code may be generated by the instruction provider 270. The code may be generated by the instruction provider 270 as a result of receiving authentication input from the caregiver.

The parameter of the patient may be measured by a sensor 250 of the external device 200. The parameter of the patient measured by the external device 200 may be compared against verified parameter data pertaining to the patient. The parameter of the patient measured by the external device by the external device 200 may also be compared against the same parameter of the patient being measured by a sensor 150 of the implant. The parameter of the patient may be a biometric parameter.

The sensors 150, 250, the parameter of the patient, as well as further methods and devices related to external device 200 authentication based on measuring parameters of the patient may be further described herein under aspect 256SE. In these cases, the implant 100 and/or external device(s) comprises the necessary features and functionality (described in the respective sections of this document). Such authentication may operate automatically, not requiring any actions to be performed by the user or patient while still providing secure authentication.

The parameter of the caregiver may, similarly to the parameter of the patient, be measured by a sensor of a device associated with the caregiver e.g. the second external device 300. The parameter of the caregiver may be compared against verified parameter data pertaining to the caregiver.

FIG. 71 shows a flow chart over methods related to relaying communication between the second external device 300 and the implant 100 implanted in the patient via the wireless transceiver 208 of an external device 200. The method may comprise the steps of: Receiving S5301, by the wireless transceiver, the instruction from the second external device communicated using the first network protocol.

Receiving S5302, by the verification unit 220, authentication input from the user.

Authenticating S5303 the relaying functionality of the external device based on the authentication input.

Upon authentication of the relaying functionality of the external device, transmitting S5304, by the wireless transceiver, the instruction to the implant, using a second network protocol Upon non-authentication or failed authentication of the relaying functionality of the external device, holding the instructions at the external device

The step of transmitting the instruction to the implant 100, when the instruction received at the external device 200 is encrypted, may be performed without decrypting the instruction at the external device 200.

The external device 200 may thus be made less complex and without decryption capability. Since the instruction is encrypted at the external device, the physical loss or theft of such an external device will not come with the loss of potentially sensitive decrypted implant instructions.

The method may further comprise the step of receiving S5305, by the instruction provider 270 of the second external device 300, instructions from the caregiver, and the step of generating S5306 at least one component of the instruction.

The instruction provided by the caregiver may comprise a decision or confirmation to run a functionality or program of the implant. The generated at least one component of the instruction may comprise actual physical actions required by the implant 100 in order to perform the desired functionality or program.

The caregiver, may provide, authentication information input comprising at least one of the code and the parameter of the caregiver. The instruction provider 270 may generate the code.

The wireless transceiver may be configured to receive the instruction from the second external device communicated using a first network protocol.

The wireless transceiver may be configured to transmit the instruction to the implant communicated using a second network protocol.

The external device may be configured to decrypt the communication from the second external device at the external device and wherein the external device may be further configured to transmit the decrypted communication to the implant via a short-range communication method. The short-range communication method may e.g. be an NFC or RFID type method.

A computer program product of, or adapted to be run on, an external device is also provided, which comprises a computer-readable storage medium with instructions adapted to make the external device perform the actions as described above.

### Aspect 254SE Automatic Update—Automatic Update of Control Program of Implant—Embodiments of Aspect 254SE of the Disclosure

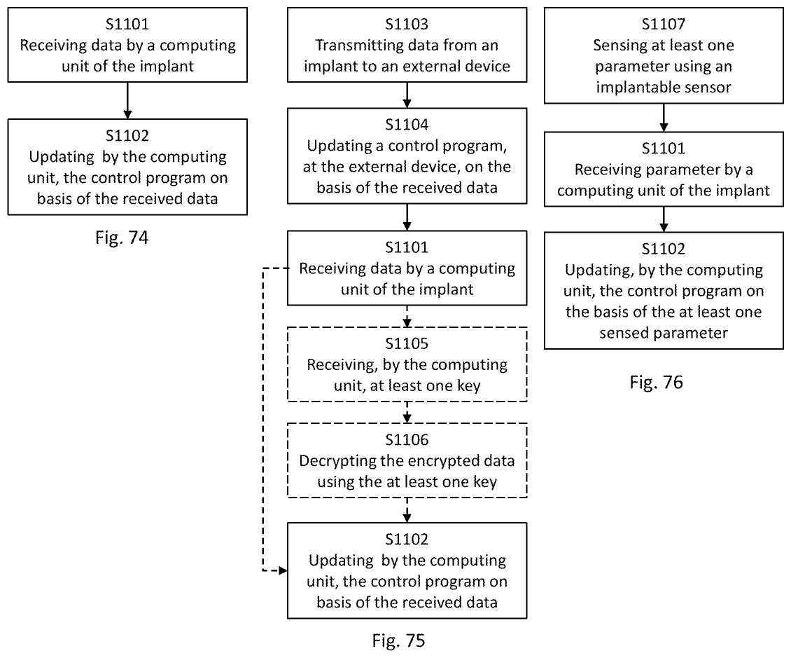

In aspect 254SE, increased security for updating a control program of an implant and associated communications between an external device(s) and the implant is provided. FIGS. 74-84 shows embodiments of this aspect.

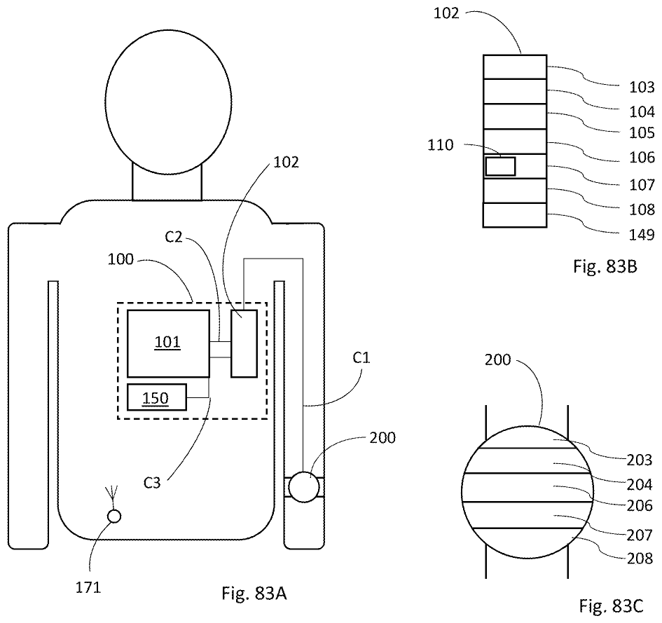

Generally, aspect 254SE defines a method, as shown in FIG. 74 in conjunction with FIGS. 83A-C and 84, for updating a control program 110 (e.g. stored in a memory 107) adapted to run in a computing unit 106 of an implant 100 when implanted in a patient. The method comprises receiving S1101 data by the computing unit, and updating S1102 by the computing unit, the control program on the basis of the received data.

The expression “updating” is intended to encompass similar terms such as adjusting, overriding, or calibrating the control program. Any updates or additions (such as installing a new software application) may be done to the control program 110 using this embodiment. A flexible approach is thus achieved, where the control unit 106 uses received S1101 data for updating S1102 the control program of the implant 100. In one embodiment, updating the control program comprises adjusting at least one parameter of the implant.

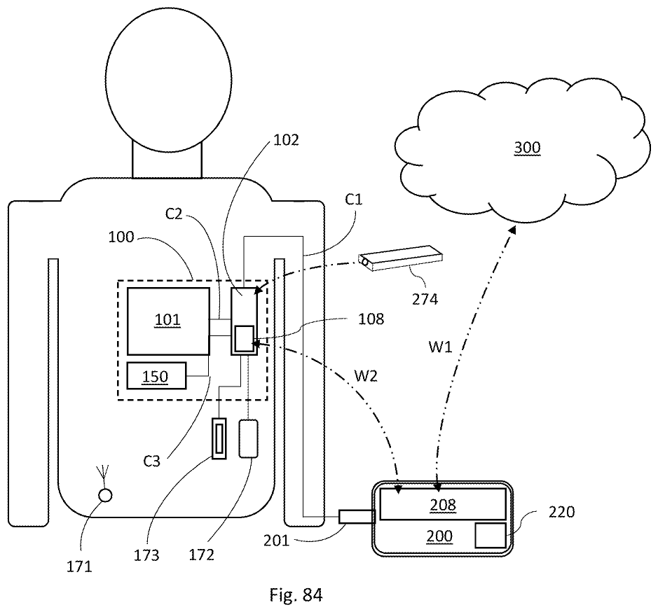

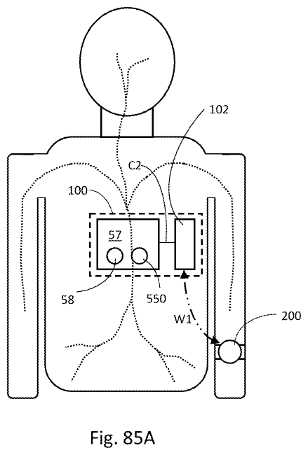

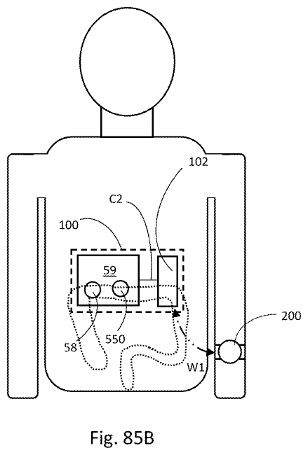

The method of FIG. 74 may be extended as shown in FIG. 75 in conjunction with FIG. 84. In this embodiment, data is transmitted S1103 from the implant 100 to an external device 200. The data may be wirelessly transmitted W2 using a wireless transceiver 108 or transmitted using conductive communication C1 using a wired transmitter 103.

Conductive communication may be achieved as described herein under aspect 247SE. In these cases, the implant and/or external device(s) comprise the necessary features and functionality (described in the respective sections of this document) for performing such conductive communication. Using a conductive communication for communication relating to updating of the control program 110 may be preferable as it makes it harder for malicious third parties to access the system and implement unauthorized control programs in e.g. implant.

In the external device, the data is received (e.g. using a wireless transceiver 208 or a wired transceiver 203. In this embodiment, the external device has the control program of the implant stored (e.g. in memory 207) and the external device 200 updates S1104 the control program (e.g. using a computing unit 206 of the external device 200) on the basis of the received data. The updated control program is then transmitted to the implant (by wire C1 or wirelessly W2) such that the data received S1101 by the computing unit 106 comprises the updated control program. The updated control program is then installed or similar in the implant. In other words, the computing unit 106 updates S1102 the control program on the basis of the received data.

In some embodiments, the updated control program transmitted to the implant 100 from the external device 200 is encrypted. In this case, the method of updating the control program at the implant 100 further comprises receiving S1105, by the computing unit 106, at least one key, and decrypting S1106 the encrypted data using the at least one key. To further increase security, the received key may be combined with a key stored at the implant 100, where the encrypted data may only be decrypted using the combined key.

Further examples and details of how to perform encryption of data transmitted between the implant 100 and the external device 200 can be found as described herein under the second, third or sixth aspect. In these cases, the implant and/or external device(s) comprises the necessary features and functionality (described in the respective sections of this document) for performing such encryption/decryption. Encryption of the communication in relation to update of the control program provides extra security in the update process as unauthorized update attempts may be disregarded and place the implant in a safe lockdown mode.

The key may be transmitted using conductive communication C1 or wireless communication W1.

Improved security may further be achieved by requiring, at the implant, that the connection used for transmission of the updated control program, i.e. the wireless W1 or wired connection C1 needs to be authenticated before the computing unit 106 updates the control program 110 on basis of the received updated control program from the external device 200. In these embodiments, upon positive authentication of the connection, the computing unit 106 updates the control program 110. If the connection is not authenticated, the computing unit may disregard the received update of the control program 110. The confirmation/authentication of the connection between the implant and the external device can be performed as described herein under the fifth, thirteenth or fifteenth aspect. In these cases, the implant and/or external device(s) comprises the necessary features and functionality (described in the respective sections of this document) for performing such confirmation/authentication.

In some embodiment, the data transmitted S1103 from the implant 100 comprises at least one physiological parameter of the patient (such as blood pressure, pulse, etc.). This embodiment is further described herein under the twelfth or thirteenth aspect. In these cases, the implant and/or external device(s) comprises the necessary features and functionality (described in the respective sections of this document) for sensing the physiological parameter. For example, in case the sensor senses that the values for blood pressure and pulse of the patient adequately match known or expected values, based on patient history or statistics, the control program may be updated S1104. Another example comprises that the sensor senses that the variability of the blood sugar values is to large, where the blood sugar value controls the amount of insulin that is ejected by the implant. In this case, the regulator algorithm used for controlling the insulin ejection of the implant may need to be updated S1104 at the external device to better suit the patient in which the implant is implanted into.

In some embodiments, the data transmitted S1103 from the implant 100 comprises at least one functional parameter (such as status of a battery, version of the control program, error log of the implant etc.) of the implant. In one embodiment, the sensor 150 senses that the power of a battery of the implant 100 is low, whereby the control program is updated at the external device such that some functionality of the implant (e.g. a feedback functionality or other features of the implant) is turned off. When the sensor 150 senses that the power has been increased, the control program may be updated such that the previously disabled functionality is enabled again.

In some embodiments, the updating of the control program 110 is performed at the implant without any external involvement. An example of such embodiment is shown in FIG. 76. In this embodiment, the method of updating the control program comprises sensing S1107 at least one parameter using an implantable sensor 150. The sensor 150 may be included in the implant or external to the implant 100 but in connection (by wire or wirelessly) with the implant. The sensed data thus constitute the received S1101 data by the computing unit 106, whereby the computing unit 106 updates S1102 the control program on the basis of the at least one sensed parameter.

For example, the sensor 150 may be for sensing at least one physiological parameter of the patient, wherein the received S1101 data by the computing unit comprises at least one physiological parameter of the of the patient. Examples of physiological parameter of the patient as further described under the twelfth or thirteenth aspect. In these cases, the implant comprises the necessary features and functionality (described in the respective sections of this document) for sensing the physiological parameter. The computing unit may then update the control program on basis of the received at least one senses parameter. For example, in case the sensor senses that senses that the variability of the blood sugar values is to large, where the blood sugar value controls the amount of insulin that is ejected by the implant. In this case, the implant may itself update S1102 the regulator algorithm used for controlling the insulin ejection of the implant may need to be updated to better suit the patient in which the implant is implanted into.

Alternatively, or additionally, the sensor 150 may be for sensing at least one functional parameter of the implant, wherein the received S1101 data by the computing unit comprises at least one functional parameter of the implant. In one embodiment, the sensor 150 senses that the power of a battery of the implant 100 is low, whereby the control program is updated by the computing unit such that some functionality of the implant (e.g. a feedback functionality or other features of the implant) is turned off. When the sensor 150 senses that the power has been increased, the control program may be updated such that the previously disabled functionality is enabled again.

The implant may be in communication with further sensors (external to the implant 100), such as an implantable sensor 171 adapted to sense at least one parameter (functional and/or physiological as described above), wherein the received S1101 data by the computing unit 106 comprises said at least one sensed parameter, wherein the computing unit 106 is configured for updating S1102, the control program on the basis of the at least one sensed parameter.

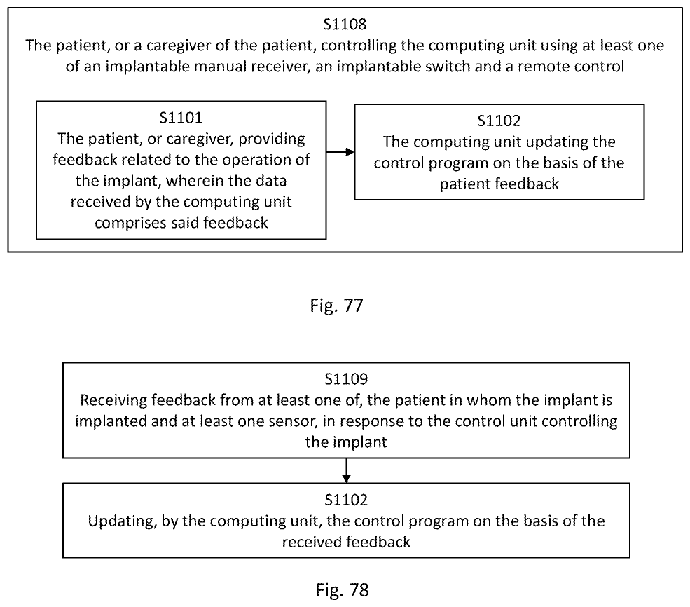

In some cases, the patient may provide input to the implant to be used for updating the control program. One example of such embodiment is shown in FIG. 77, which comprises the patient, or a caregiver of the patient, controlling S1108 the computing unit 106 using at least one of an implantable manual receiver 172, an implantable switch 173 and a remote control 274, the patient, or caregiver, providing feedback related to the operation of the implant, wherein the data received S1101 by the computing unit comprises said feedback. The computing unit 106 updates S1102 the control program on basis of the patient feedback.

The switch 173 may be a reset function or switch 173. Further information and details around the reset switch 173 and other involved devices and processes for handling such reset switch 173 may be performed as described herein under the aspect 244SE. In these cases, the implant and/or external device(s) comprises the necessary features and functionality (described in the respective sections of this document) for performing such a resetting of the implant 100.

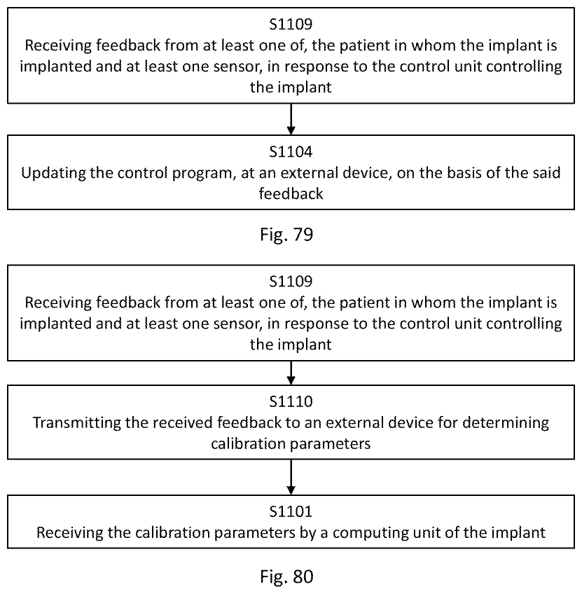

In some embodiments shown in FIG. 79, the method of updating the control program 110 of the implant 100 comprises, receiving S1109 feedback from at least one of, the patient in whom the implant is implanted and at least one sensor 150, 171, in response to the control program controlling the implant, and updating S1102, by the computing unit, the control program on the basis of the received feedback. The updating process may thus be iterative, where an update S1102 result in new data received S1101 by the computing unit 106 as a result of the update (e.g. patient feedback relating to the updated functionality of the implant 100, or sensor data relating to the updated functionality of the implant 100), whereby the control program 110 is again updated S1102.

The iterative update may also involve the external device 200. One example of this embodiment is shown in FIGS. 79-80. For example, the feedback from at least one of, the patient in whom the implant is implanted and at least one sensor 150, 171, in response to the control program controlling the implant may be received directly by the externa device 200 or transmitted from the implant 100 to the external device 200, which updates S1104 the control program on the basis of the said feedback, wherein the data received S1101 by the computing unit comprises the updated control program. In another embodiment, the received S1101 data by the computing unit 106 comprises calibration parameters transmitted from the external device 200, said calibration parameters based on the feedback provided to the external device.

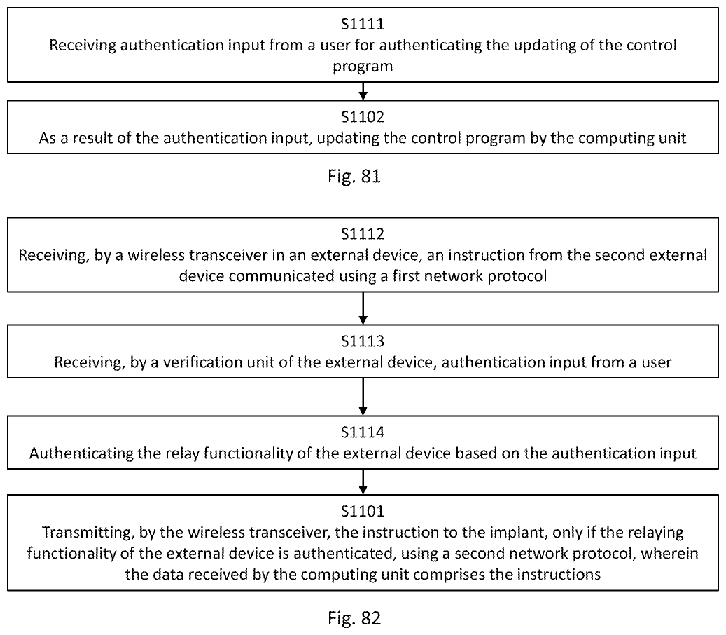

In some embodiments, shown in FIG. 81, authentication input from a user is required to update the control program. In these embodiments, the external device comprises an interface for inputting authentication data, e.g. using a verification unit 220 of the implant. The method of updating the control program 110 of the implant 100 thus may comprise receiving S1111 authentication input from a user for authenticating the updating of the control program, and as a result of the authentication input, updating S1102 the control program by the computing unit. In other words, upon valid authentication, the control program 110 may be updated. The authentication input may comprise a code, a biometric input (fingerprint, iris scanner etc.) or any other suitable means for authentication. In some embodiments, the updated control program, or calibration parameters, etc., may not be transmitted from the external device unless valid authentication is inputted. In other embodiments, the control unit 106 may not update, install, or calibrate the control program 110 in the implant 100 unless a valid authentication is determined. The implant 100 may comprise the data needed for determining if the authentication is valid or not (i.e. the correct code, the approved fingerprints etc.). In other embodiments, the external device 200 may comprise the data needed for determining if the authentication is valid or not (i.e. the correct code, the approved fingerprints etc.).

In some embodiments, a second external device 300 is involved in the updating of the control program. The second external device 300 may be controlled by medical staff, manufacturer of the implant 100 or any other suitable individual or organization for updating the control program 110. In this embodiment, the implant 100 is wirelessly connected to an external device 200, the external device configured to relay communication between a second external device 300 and the implant 100. A system of the implant 100, the external device 200 and the second external device 300 is thus formed. The external device 200 comprising a wireless transceiver 208 configured for wireless communication W1, W2 with the second external device and the implant, the wireless transceiver 208 configured to receive S1112 an instruction (using the wireless communication W1) from the second external device 300 communicated using a first network protocol, wherein the wireless transceiver 208 is configured to transmit (using the wireless communication W2) the instruction to the implant 100 using a second network protocol.

The relaying functionality may in some embodiments need to be authenticated by the user/holder of the external device. In these embodiments, the method for updating the control program 110 comprises receiving S1113, by the verification unit 220 of the external device 200, authentication input (code, parameter of the patient, etc. as described herein) from a user, authenticating S1114 the relay functionality of the external device based on the authentication input. In these embodiments, the wireless transceiver 208 transmits the instruction to the implant, only if the relaying functionality of the external device is authenticated, using a second network protocol, wherein the data received S1101 by the computing unit 108 comprises the instructions. The computing unit 106 may then update S1102 the control program 110 accordingly.

The instructions received S1112 from the second external device may comprise one of the updated control program, and calibration parameters of the implant. In some embodiments, not shown in the figures, feedback and/or sensed parameters are transmitted from the implant/external device to the second external device 300 prior to the wireless transceiver 208 receives S1112 the instruction from the second external device 300. The second external device 300 may thus base the instructions on such received data.

The first network protocol may a standard network protocol from the list of:
a Radio Frequency type protocol
a RFID type protocol
a WLAN type protocol
a Bluetooth type protocol
a BLE type protocol
a NFC type protocol
a 3G/4G/5G type protocol
a GSM type protocol

The second network protocol may be a proprietary network protocol.

More embodiments describing network protocols may be implemented as described herein under aspect 250SE. In these cases, the implant and/or external device(s) comprises the necessary features and functionality (described in the respective sections of this document) for performing transmission of data.

The routing may be performed as described herein under aspect 253SE. In these cases, the implant and/or external device(s) comprises the necessary features and functionality (described in the respective sections of this document) for performing such routing.

Further information and definitions of features, functionality of aspect 254SE can be found in this document in conjunction with the other aspects.

A first communication system may be used for receiving data by the computing unit 106 of the implant 100. A second communication system may be used for transmitting data from the implant 100 to the external device 200.

The method may further comprise relaying data to the second external device 300 and receiving the updated control program at the second external device 300.

A caregiver may transmit data to the implant 100 from a second external device 300 directly or via the external device 200.

According to embodiments of the first part of aspect 254SE a connection between the implant 100 and the external device 200 is authenticated by a conductive communication or connection between the implant 100 and the external device 200.

This feature may be achieved as described herein under aspect 247SE. In these cases, the implant and/or external device(s) comprises the necessary features and functionality (described in the respective sections of this document) for performing such conductive communication. The communication may thus be provided with an extra layer of security in addition to the encryption by being electrically confined to the conducting path e.g. external device 200, conductive member 201, conductive connection C1, implant 100, meaning the communication will be excessively difficult to be intercepted by a third party not in physical contact with, or at least proximal to, the patient.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

### Aspect 255SE Information from Implant—Information from Implant—Embodiments of Aspect 255SE of the Disclosure

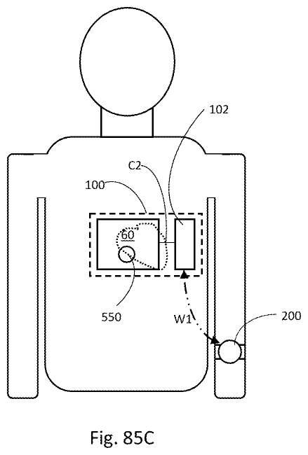

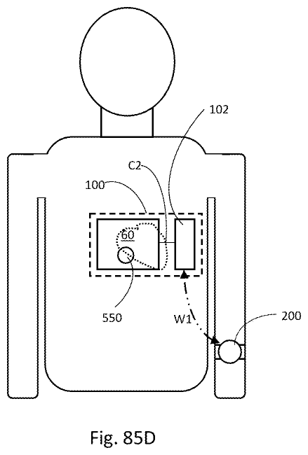

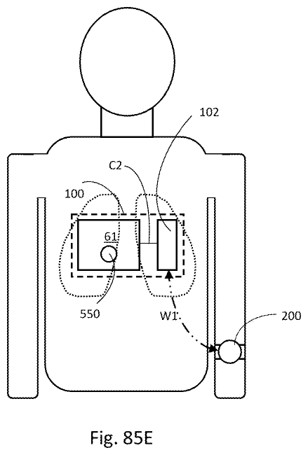

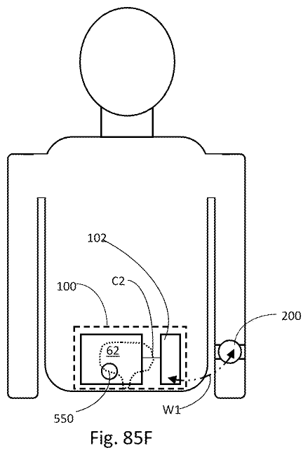

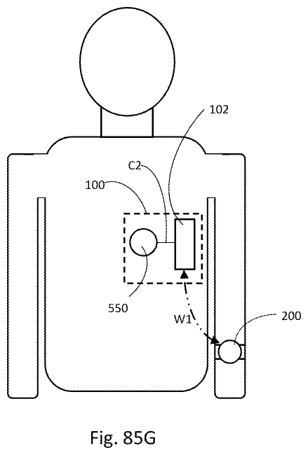

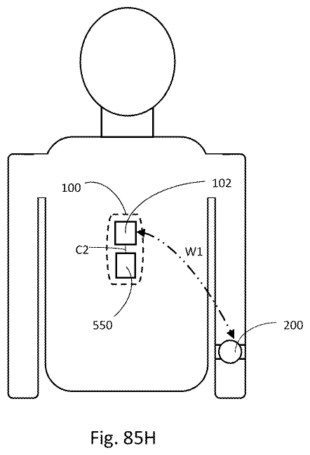

In aspect 255SE, communication of sensor parameters between an implant and an external device is provided. FIGS. 85A-H show embodiments of this aspect.

FIGS. 85A-H shows an implant 100 implanted in various places of a patient's body. It is to be understood that the implant 100 could be placed anywhere in the patient's body and is not restricted to the placements shown in FIGS. 85A-H.

FIG. 85A shows an implant 100 in the abdominal area of a patient's body. The implant 100 comprises a vascular portion 57, adapted to be placed in proximity to a blood vessel. In FIG. 85A the vascular portion 57 is placed in the abdominal area, but it could be placed near any blood vessel in the body, such as for example the aorta, common carotid artery, subclavian artery, common iliac arteries, subclavian vein, inferior vena cava, renal veins, common iliac vein, and pulmonary arteries. The vascular portion 57 further comprises a sensor 550. In FIG. 85A, the implant 100 comprises one sensor 550, it is however plausible to have more sensors. The sensor 550 can sense physiological parameters of the patient, such as for example blood pressure and temperature. The sensor 550 in FIG. 85A is configured to sense at least one parameter related to the blood of the patient. The implant 100 also comprises a communication unit 102. The communication unit 102 may comprise different means for communication, for example a wireless transceiver, and/or a wired transceiver. In the following, wireless communication is used by way of example. The communication unit 102 is connected to the vascular portion 57 by a connection C2 (it should be noted that wired, or wireless connection may be employed between the implant 100 or the communication unit 102 and the sensor 150, further described herein with reference to the other aspects). The communication unit 102 can wirelessly (or by wire) transmit W1 the parameter sensed by the sensor 550 to an external device 200. The external device could be any device external to the body, in some embodiments, handheld and easily accessible. The external device 200 can have a display on which information regarding the sensed parameter can be presented to the patient. This information can be presented in various ways, such as displaying the measured value of the parameter, or displaying a phrase or color code, or any other information that guides the patient on how to proceed. The vascular portion 57 can comprise a needle (not shown), with which blood can be extracted from the blood vessel to the sensor 550. In FIG. 85A, the vascular portion 57 comprises a needle operating device 58. The needle operating device 58 can operate the needle such that it can extract blood at a first site, and then change and extract blood at a second site. One advantage of this is that unnecessary damage due to repeated punctures at the same site of the vessel can be avoided or minimized. It is further possible that the sensor 550 is an optical sensor that could sense optical parameters of the blood. The optical sensor 550 could in such case also be configured for spectrophotometry. The sensor 550 could also further sense visible light, UV light and/or IR radiation. It is further possible that the sensor 550 in FIG. 85A can sense a parameter relating to at least one of the following: oxygen saturation, blood pressure, a function of the liver, the existence of cancer, the bile function, glucose, lactate, pyruvate, prostate-specific antigen, cholesterol level, potassium, sodium, cortisol, adrenalin, ethanol, blood composition, platelets, white blood cells, red blood cells, viscosity, flux, the direction of flow, flow velocity, blood plasma concentration, hormones, enzyme activity, calcium, iron, iron-binding capacity, transferrin, ferritin, ammonia, copper, ceruloplasmin, phosphate, zinc, magnesium, pH, oxygen partial pressure, carbon dioxide, bicarbonate, protein(s), blood lipids, tumor markers, vitamins, toxins, antibodies, electrolytes. The sensor 550 could also sense a parameter related to the effect of a therapeutic treatment, or the presence of a pharmaceutical. If for example the patient has consumed a too high a dose of a medicine (or, equally, too low) the sensor 550 senses this and the communication unit 102 can communicate this to the external device 200. This makes the patient, doctor or any other caretaker aware of the dose issue and enables them to adjust the dose accordingly. The sensor 550 could further sense the presence of at least one of the following: an antibiotic pharmaceutical, a chemotherapy pharmaceutical and insulin. If the implant 100 is utilized by diabetes patients, the sensor 550 senses the insulin levels in the blood and communicates this to the external device 200. In cases where the external device 200 is a watch, smart phone or any other easily accessible device, the patient can immediately, and discretely, get information on how to act. This direct and reliable information ensures that the patient doesn't have to guess or estimate the dose needed, thus avoiding the risks of over or underdosing. The sensor 550 could also be configured to sense a parameter related to cancer treatments and/or antibiotic treatments.

FIG. 85B shows another embodiment of aspect 255SE. In this embodiment, the implant 100 comprises a food passageway portion 59 which is placed in proximity to the food passageway of a patient. The sensor 550 can sense at least one of intestinal activity, activity of the stomach or activity of the esophagus and can be for example an accelerometer, a motility sensor and/or a strain sensor. It is also plausible that the sensor 550 can sense an electrical parameter. In some cases, the sensor 550 can also sense contents in the esophagus, the stomach or in an intestine. It is possible that the sensor 550 in FIG. 85B comprises a needle, with which contents in the food passageway can be extracted and transported to the sensor 550. In this case, the food passageway portion 59 comprises a needle operating device 58 which can displace the needle such that it can change from extracting contents at a first site of the food passageway to extracting contents at a second site of the food passageway. The sensor 550 could also be an optical sensor and configured to utilize spectrophotometry. The optical sensor 550 can sense visible light, UV light and/or IR radiation. It is also possible that the sensor 550 in FIG. 85B is an audio sensor adapted to sense sound. When the hollow intestines move, they produce sounds that can give information on a patient's condition. The audio sensor 550 could for example be utilized for determining ileus, a condition in which there is a lack of intestinal activity and thus effects the sounds coming from the bowel. In some cases, the bowel sounds are unable to be heard by the patient and/or medical professional trying to listen and considering that acute abdominal diseases is a prominent death cause if left untreated, the audio sensor 550 is a very powerful tool. In these cases, the audio sensor 550 picks up and communicates via the communication unit 102 information to the patient and/or doctor. The food passageway is to be understood as the entire length through which the food passes, notably the moth, the esophagus, the stomach, the intestines, and the rectal region.

FIG. 85C shows an embodiment in which the implant 100 comprises an ultrasound sensor 550. In FIG. 85C, the ultrasound sensor 550 is placed in a cardiac portion 60 of the implant 100. The cardiac portion 60 is placed near the patient's heart, it is however equally possible that the implant 100 is placed in another part of the patient's body, and that the ultrasound sensor 550 is in proximity to another body part. The ultrasound sensor 550 in FIG. 85C can sense the blood flow in the heart. Should for example the blood flow suddenly decrease, myocardial ischemia occurs, which can lead to a heart attack. Silent myocardial ischemia is a condition in which the patient doesn't experience any noticeable signs or symptoms. Such condition could therefore be avoided by utilizing the sensor 550 of FIG. 85C. The ultrasound sensor 550 could also sense presence of fluid in the body of the patient, for example in the pericardial cavity. It could also sense the level of urine in the urinary bladder, in which case the sensor 550 and implant 100 is placed in proximity to the renal system.

FIG. 85D shows an embodiment of aspect 255SE in which the implant 100 comprises a cardiac portion 60 and has a sensor 550 configured to sense a parameter related to the patient's heart. The sensor 550 could for example sense a parameter related to the electrical activity of the heart. It could also be adapted to sense a sound parameter related to the heart. In these cases, the implant comprises the necessary features and functionality for performing such sensing.

FIG. 85E shows an implant 100 with a pulmonary portion 61. The pulmonary portion 61 comprises the sensor 550 and is located in proximity to the patient's lungs. The sensor 550 is adapted to sense parameters relating to the lungs. The sensor 550 could for example sense respiratory activity. In this case, the sensor 550 could be an accelerometer, a motility sensor and/or a strain sensor. It is also possible that the sensor 550 I FIG. 85F is an optical sensor, and/or an audio sensor.

FIG. 85F shows an implant 100 being implanted in proximity to the renal system. The implant 100 comprises a urinary portion 62 and a sensor 550 which senses parameters relating to the urine bladder of the patient. It is possible that the sensor 550 is an optical sensor, and that it senses activity of the urinary bladder. The sensor 550 in FIG. 85F could also be an accelerometer, a motility sensor, and/or a strain sensor.

FIG. 85G shows an implant 100 placed in the upper abdominal area of a patient. It is to be noted that the implant 100 could equally be placed in other parts of the body. The implant 100 in FIG. 85G has an audio sensor 550 which sense audio parameters of the patient. The audio parameter could pertain to at least one of: the activity of the gastrointestinal system, the activity of the lungs, the activity of the heart, and the patient's voice. One way of utilizing the patient's voice (or any other sound or person's voice) is to compare the received audio in the implant 100 with the received audio in the external device 200. The comparison can then be used to synchronize the implant 100 with the external device 200.

FIG. 85H shows an implant 100 ingested by the patient. The ingested implant 100 has an audio sensor 550 which can sense audio parameters. The audio parameter could pertain to at least one of: the activity of the gastrointestinal system, the activity of the lungs, the activity of the heart, and the patient's voice.

It is to be understood, that any and all of the sensor(s) described herein with reference to FIGS. 85A-H are also compatible with, and could be integrated in, the embodiments pertaining to the first, second, third, fourth, fifth, sixth, seventh, eighth, ninth, tenth, eleventh, thirteenth and fifteenth aspects. In these cases, the implant and/or external device(s) comprises the necessary features and functionality (described in the respective sections of this document) for performing such communication. It is also noted that the communication, wireless or electrical, between the sensor(s) described with reference to FIGS. 85A-H and any external device may be performed as described herein under the first, second, third, fourth, fifth, sixth, seventh, eighth, ninth, tenth, eleventh, thirteenth and fifteenth aspects. In these cases, the implant and/or external device(s) comprises the necessary features and functionality (described in the respective sections of this document) for performing such communication. It is to be noted, however, that even though the communication between the communication unit 102 and the external device 200 is wireless by way of example in the FIGS. 85A-H, it is plausible that the communication is electrical or by any other means wired. Further, the communicated and/or encrypted information referred to herein in the second, third, sixth, seventh, ninth, tenth, and thirteenth aspects can pertain to the sensed parameters described with reference to FIGS. 85A-H.

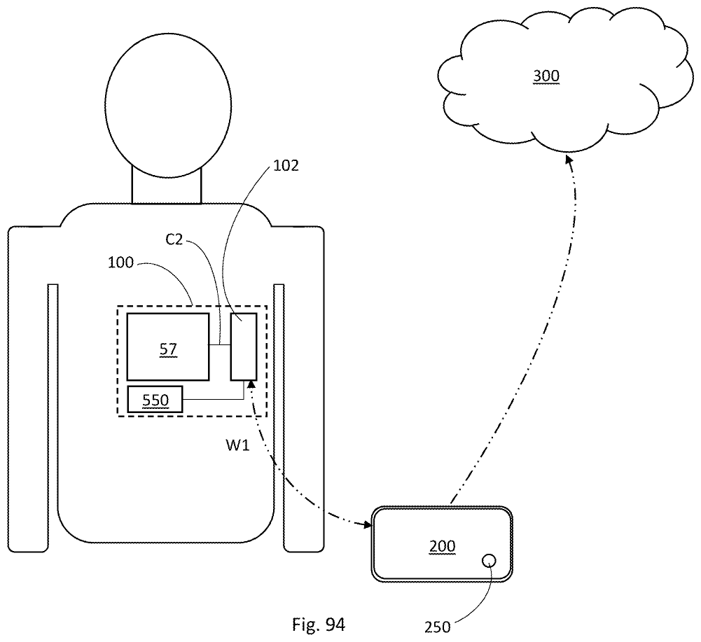

FIG. 94 illustrates a system comprising an implant 100, implanted in a patient, an external device 200, and a second external device 300. The external device 200 is configured to transmit data pertaining to the sensed parameter to the second external device 300. The external device 200 is configured to add information to the data pertaining to the sensed parameter before transmitting to the second external device 300.

The information added may comprise at least one of:
a weight of the patient,
a height of the patient,
a body temperature of the patient,
eating habits of the patient,
physical exercise habits of the patient,
toilet habits of the patient,
an outside or external temperature of the patient, and
geographic position data of the patient.

The external device 200 may comprise a sensor 250 for recording the information to be added to the data pertaining to the sensed parameter. The sensor 250 may comprise a thermometer, e.g. for measuring a body temperature of the patient, or a geographical positioning sensor such as a global navigation satellite system, GNSS, receiver, e.g. for recording geographic position data of the patient.

The external device 200 may be configured to automatically add the information to the data pertaining to the sensed parameter. The external device may alternatively or also be configured to add information to the data pertaining to the sensed parameter upon receiving a manual input from a user. Such a manual input may relate to the information added such as e.g. a weight of the patient. Input may be performed by the patient interacting with a user interface of the external device. Such a user interface may comprise a display and/or a keypad. The manual input may comprise authentication or verification of the user to transmit automatically provided data. Authentication may be established by the patient inputting a code or providing a biometric input such as e.g. a fingerprint to a fingerprint reader/sensor of the external device 200.

The second external device 300 may e.g. be a device controlled by a healthcare provider of the patient or a provider/manufacturer of the physical implant.

The implant 100, the external device 200, the second external device 300, and the communication between these may be further described in the other aspects of this document. Authentication and verification may also be further described in the other aspects of this document.

Further information and definitions of the wireless connection W1, the electrical connection C2 and the external device 200 can be found in this document in conjunction with the aspect 244SE and the general definition of features used in this disclosure.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

### Aspect 256SE Device Synchronization Patient Parameter—Authenticating a Connection Between an Implant and the External Device Using a Patient Parameter—Embodiments of Aspect 256SE of the Disclosure

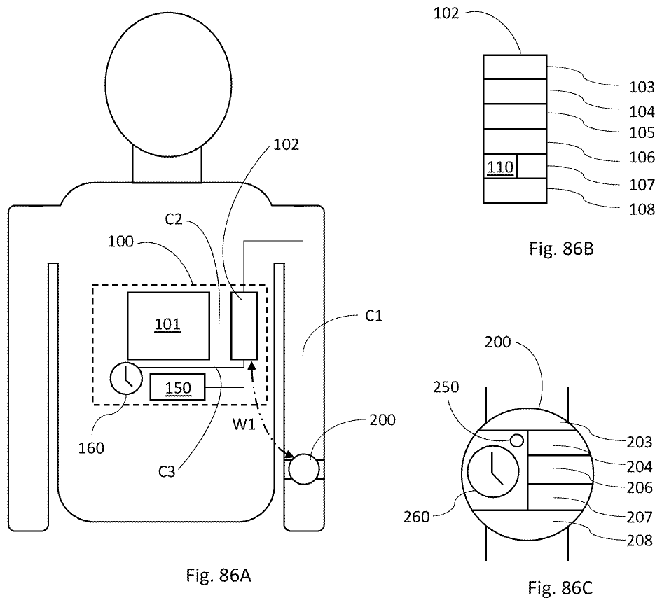

In aspect 256SE, increased security for communication between an external device(s) and an implant is provided. FIGS. 86-89 shows embodiments of this aspect.

FIGS. 86A-C and 87 show an implant 100 implanted in a patient and an external device 200.

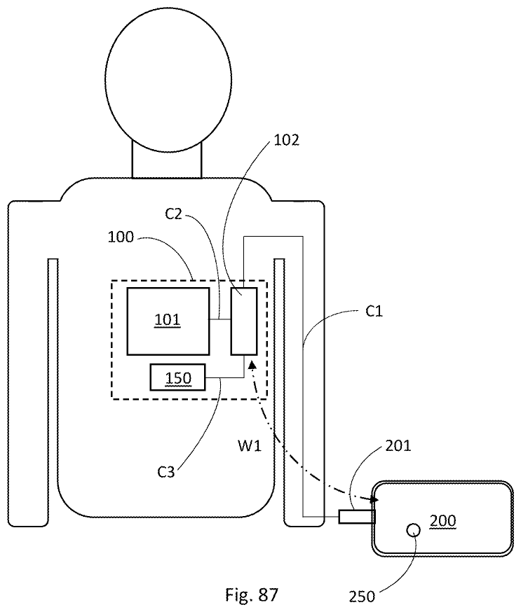

The implant 100 may comprise a first sensor 150 for measuring a parameter of the patient by the implant 100. The first sensor 150 may be configured to measure a pulse of the patient, a respiration rate of the patient, a temperature of the patient, at least one sound of the patient, or at least one physical movement (e.g. an acceleration of the physical movement) of the patient. The first sensor 150 may comprise a microphone, a thermometer, an accelerometer, a gyroscopic sensor, a pressure sensor, or a flow sensor. The first sensor may be wired or wirelessly connected to the implant. In FIGS. 86-87, a wired (electrical) connection C3 is shown by way of example. Further example of sensors may be found herein under aspect 255SE. As such, the communication may serve a dual purpose of transmitting the information for purposes such as e.g. collection and analysis as well as authentication of the connection.

The external device 200 may comprise a second sensor 250 for measuring a parameter of the patient by the external device 200. The second sensor 250 may be configured to measure a pulse of the patient, a respiration rate of the patient, a temperature of the patient, at least one sound of the patient, or at least one physical movement (e.g. an acceleration of the physical movement) of the patient. The second sensor 250 may comprise a microphone, a thermometer, an accelerometer, a gyroscopic sensor, a pressure sensor, or a flow sensor. The second sensor 250 may be wired or wirelessly connected to the external device 200. The second sensor 250 may be integrally a part of the external device 200 (as is shown in FIGS. 86-87) or a physically separate unit from but connected to the external device 200. An example of a separate second sensor 250 may be a bracelet. Such a bracelet second sensor 250 may be adapted to measure a pulse at a wrist of the patient and communicate data or metrics pertaining to the measurement to the external device 200 conductively by a wire or wirelessly.

The second sensor 250 should correspond to the first sensor 150 in that comparable sensed parameters should be measured by the sensors 150, 250.

The implant 100 may further comprise an internal computing unit 106. The internal computing 106 unit may be configured for receiving a parameter of the patient, from the external device 200. The internal computing unit 106 may be further configured for comparing the parameter measured by the implant 100 to the parameter measured by the external device 200. The internal computing unit 106 may be further configured for performing authentication of the connection based on the comparison.

The external device may further comprise an external computing unit 206. The external computing unit 206 may be configured for receiving a parameter of the patient, from the implant 100. The external computing unit 206 may be further configured for comparing the parameter measured by the external device 200 to the parameter measured by the implant 100. The external computing unit 206 may be further configured for performing authentication of the connection based on the comparison.

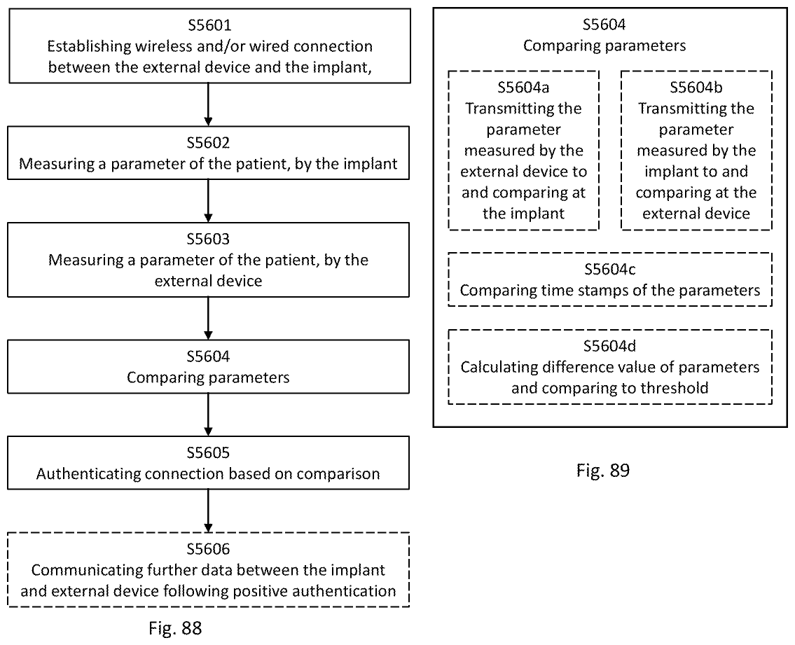

FIGS. 88 and 89 show flow charts of methods for authenticating a connection between an implant 100 implanted in a patient, and an external device 200. The method may comprise the step of establishing a connection S5601 between the external device 200 and the implant 100. The method may further comprise the step of measuring a parameter S5602 of the patient, by the implant 100. The method may further comprise the step of measuring the parameter S5603 of the patient, by the external device 200. The method may further comprise the step of comparing the parameter S5604 measured by the implant 100 to the parameter measured by the external device 200. The method may further comprise the step of performing authentication S5605 of the connection based on the comparison.

The method may ensure that authentication may not occur unless parameters of the patient measured by the external device and the implant match. In effect, this may prevent, or at least reduce the risk of, unauthorized connection or communication to and from the implant or the external device. Another advantage of the method is that the authentication may be performed automatically with the external device and the implant communicating with each other without requiring any verification or input authentication from the patient.

The parameter of the patient may be measured by the sensors 150, 250. The measurement may comprise recording a parameter event such as e.g. a movement detected by the sensors 150, 250 being an accelerometer. A strength or power of the parameter measured may need to exceed a set threshold value of the sensors 150, 250 for an event to be recorded. This approach may be considered a digital approach where an event is either recorded or not. As an alternative, data pertaining to the measured parameter may be recorded, e.g. a value for an acceleration of the movement detected by the sensors 150, 250 being an accelerometer as in the previous example. This may be considered a more analogue approach to measuring the parameters.

The sensors 150, 250 may also record the parameter of the patient for a set amount of time resulting in a time sequence of data pertaining to the parameter. The comparison (made by the internal 106 or external 206 computing unit) may comprise a comparison of the time sequences recorded by the external device 200 via the second sensor 250 and the implant 100 via the first sensor 150. Time sequence duration may preferably be in the range 0.01-60s, more preferably 0.1-30s, and most preferably 1-10s.

The parameter of the patient may be related to biological processes that are largely out of the patient's control such as a pulse, a respiration rate, or a temperature. Alternatively, the parameter of the patient may be controllable to the patient and configured to register for specific actions of the patient.

An example of such a controllable action may include shouting, or by other means, producing a loud sound such that both sensors 150, 250, being in relatively close proximity to each other, are able to measure. Another example of an action of the patient may be a jump or a spin such that both sensors 150, 250, located implanted inside and carried externally by the patient, may measure the same physical movement related to the jump or spin.

A relative momentary vertical acceleration exceeding 1 g (i.e. 9.8 ms\hat{ }−2) in a normal direction of a surface, on which the patient is located, may be measured by an accelerometer in order to register a jump of the patient. Relative in this context means relative or in addition to ever present or inherent accelerations such as a 1 g acceleration due to gravity. The first and second sensor 150, 250 may record substantially the same acceleration. Differences may be dependent on how structurally fixed the accelerometers are. For example, if the external device 200 with the second sensor 250 is located in a pocket of a clothing item of the patient, some extra inertia may be expected. Such differences may be accounted for by calibration of the implant 100 and the external device 200 with their respective sensors 150, 250. Similar actions for calibration may be performed regardless of the types of parameters or sensors used.

A noise level exceeding 70 dB may be measured by a microphone in order to register a sound of the patient. Due to attenuation, a noise level of an external sound may be reduced upon reaching the implant implanted internally in the body of the patient. The reduction may be accounted for by calibration of the implant 100 and the external device 200 with their respective sensors 150, 250.

The pulse, heart rate, or cardiac rate of the patient may refer to a rate or frequency at which a heart cycles through its steps for pumping blood through a cardiovascular system of the patient. The cardiovascular system, vascular system, circulatory system may comprise the heart and blood vessels such as veins and arteries of the patient. The pulse of the patient may be characterized by the frequency of peaks or troughs of a pressure of a blood flow at a specific location in the cardiovascular system. A peak of the blood flow pressure exceeding 90 mmHg may be measured by a pressure sensor in order to register a peak of the pulse of the patient.

The parameter of the patient may also comprise a blood pressure of the patient.

The blood pressure of the patient, comprising a systolic pressure (peak) and a diastolic pressure (trough) may be measured similarly to the pulse and used for authenticating the connection between the implant 100 and the external device 200.

FIG. 89 specifically relates to various ways of performing the step of comparing the parameters S5604. The parameter of the patient measured by the external device 200 may be transmitted to and used for the comparison S5604a at the internal computing unit 106 of the implant 100. The parameter of the patient measured by the external device 200 may alternatively be transmitted to the external computing unit 206 for comparison. The parameter of the patient measured by the implant 100 may be transmitted to and used for the comparison S5604b at the external computing unit 206 of the external device 200. The parameter of the patient measured by the implant 100 may alternatively be transmitted to the internal computing unit 106 for comparison.

The parameters measured by the implant 100 or the external device 200 may be provided with a timestamp. The comparison of the parameter measured at the implant 100 to the parameter measured by the external device 200 may comprise comparing S5604c the corresponding timestamps. The timestamps may comprise a time related to the measurement of the parameter of the patient e.g. a time for initializing the measurement. The timestamp may be encoded at the implant 100 or the external device 200. The timestamp may be communicated between the implant 100 and the external device 200 encoded and be decoded at the receiving end of the two.

The implant 100 may further comprise a clock 160. The external device 200 may comprise a clock 260. The clocks 160, 260 may be configured for synchronization with each other. The methods of authentication may comprise the step of synchronizing the clocks 160, 260. The clocks 160, 260 may be configured to provide the timestamp to the parameters measured by the implant 100 and the external device 200. The clocks 160, 260 may comprise a crystal oscillator.

The comparison may be performed by either the internal computing unit 106 or the external computing unit 206 calculating and comparing S5604d a difference value between the parameters measured by the implant 100 and the parameter measured by the external device 200. The internal computing unit 106 or the external computing unit 206 may be configured to authenticate the connection if the difference value is less than a predetermined threshold value, and not to authenticate the connection if the difference value equals or exceeds the predetermined threshold difference value.

The threshold difference may refer to a threshold for the difference in time between the timestamp of the parameter measured by the implant 100 and timestamp of the parameter measured by the external device 200. The threshold difference value may be a percentage value of how much the parameters or time sequence of the parameters match. The threshold difference value may preferably be in the range 50-100%, more preferably 75-100%, and most preferably 90-100%.

The communication between the implant 100 and the external device 200 may be a wireless communication using a wireless connection W1. The communication between the implant 100 and the external device may be a conductive communication using a conductive connection C1. The implant 100 and the external device 200 may be configured for wireless and conductive communication accordingly.

FIG. 87 shows the external device 200 comprising a conductive member 201 configured to be in electrical connection with the external device 200, wherein the conductive member 201 is configured to be placed in electrical connection with a skin of the patient for conductive communication with the implant 100. The method of authenticating the connection may comprise placing the conductive member 201, configured to be in electrical connection with the external device 200, in electrical connection with a skin of the patient for conductive communication with the implant 100.

Further information and definitions of conductive communication and the conductive member 201 can be found in this document in conjunction with aspect 247SE. In these cases, the implant and/or external device(s) comprises the necessary features and functionality (described in the respective sections of this document) for performing such conductive communication.

The implant 100 and the external device 200 may be configured to communicate further data S5606 between each other following positive authentication. The further data may comprise data sensed by the sensor 150 or another sensor connected to the implant 100. The further data may comprise data for updating a control program 110 running in the implant 100. The further data may comprise operation instructions for operating the implant 100. The further data may be communicated from the implant 100 to the external device 200. The further data may comprise data sensed by the sensor 150 connected to the implant 100. The further data may be encoded as described herein under the second, third and sixth aspect. In these cases, the implant and/or external device(s) comprises the necessary features and functionality (described in the respective sections of this document) for performing such encoding. The implant 100 and/or the external device 200 may be configured to determine a cryptographic hash based on the measured parameter measured by at least one of the implant 100 and/or the external device 200.

The cryptographic hash may then be comprised in all future data or at least all future data during a data transfer session. Accordingly, the implant 100 and/or the external device 200 may be configured to verify the further data based on the cryptographic hash.

The cryptographic hash is determined based on the measured parameter and is represented with a sequence of symbols such as a number. For instance, the cryptographic hash is a number indicating the pulse or oxygen saturation of the patient. Each time data is conveyed between the implant and the external device the cryptographic hash is included in the conveyed data and the external and/or the implant can thereby authenticate or verify the data by the presence of the correct cryptographic hash in the data. The cryptographic hash may be included in all data conveyed during a predetermined time interval or for a predetermined number of data transmissions which form the communication session. Upon completion of a communication session the method of aspect 256SE is repeated an a new cryptographic hash may be determined and used during a subsequent communication session.

In some implementations, the measured by the external device 200 or implant 100 is transmitted to the other one of the external device or implant wherein the comparison is performed in the one of the external device and the implant which received the transmitted measurement. The cryptographic hash may be based on the transmitted measured parameter only which is accessible at both of the implant and the external device after transmission of the measured parameter from one to the other. The cryptographic hash is determined only in response to the comparison resulting in an authentication of the connection (e.g. connection if the difference value is less than a predetermined threshold difference value).

The method for authenticating the connection may further comprise, if the comparison is performed by the implant 100, continuously requesting by the external device 200, or receiving at the external device 200, information of an authentication status of the connection between the implant 100 and the external device 200, and upon determining, at the external device 200, that the connection is authenticated, transmitting further data from the external device 200 to the implant 100. In this context, continuously may refer requesting by or receiving at the external device 200 at fixed intervals of e.g. 5, 10, 60 s. The clocks 160, 260 may be used to keep the timing of the intervals.

Authentication of the connection between the implant 100 and the external device 200 may be performed automatically without input, authentication, or verification from a user or patient. This is because the comparison of parameters measured internally and externally, by the internal and external sensors 150, 250 respectively may be enough to authenticate the connection. This may typically be the case when the parameter of the patient is related to an automatically occurring physiological function of the patient such as e.g. a pulse of the patient. Certain types of authentication may however require actions from the patient, e.g. having the patient perform specific movements.

The method for authenticating the connection may further comprise, if the comparison is performed by the external device 200, continuously requesting by the implant 100, or receiving at the implant 100, information of an authentication status of the connection between the implant 100 and the external device 200, and upon determining, at the implant 100, that the connection is authenticated, transmitting further data from the implant 100 to the external device 200. In this context, continuously may refer. requesting by or receiving at the implant 100 at fixed intervals of e.g. 10s. The clocks 160, 260 may be used to keep the timing of the intervals.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

A computer program product of, or adapted to be run on, an external device is also provided, which comprises a computer-readable storage medium with instructions adapted to make the external device perform the actions as described above.

Further information and definitions can be found in this document in conjunction with the other aspects.

### Aspect 257SE Device Synchronization Sensation Unit—Sensation Unit for Authenticating a Connection Between an Implant and the External Device—Embodiments of Aspect 257SE of the Disclosure

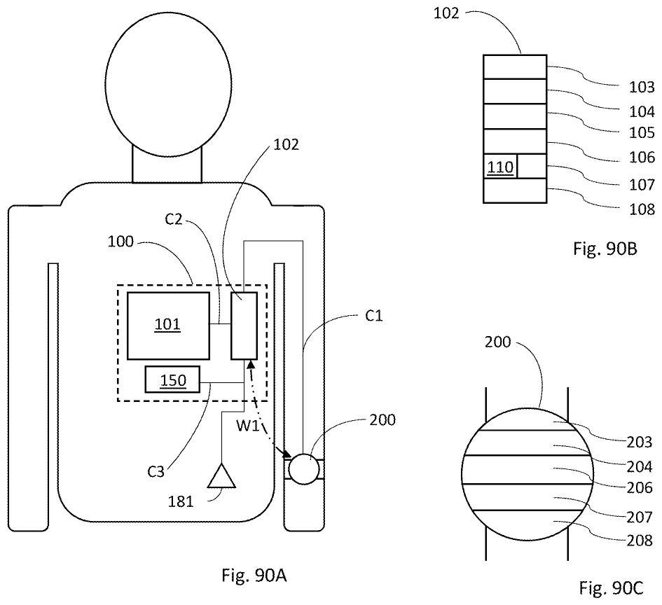

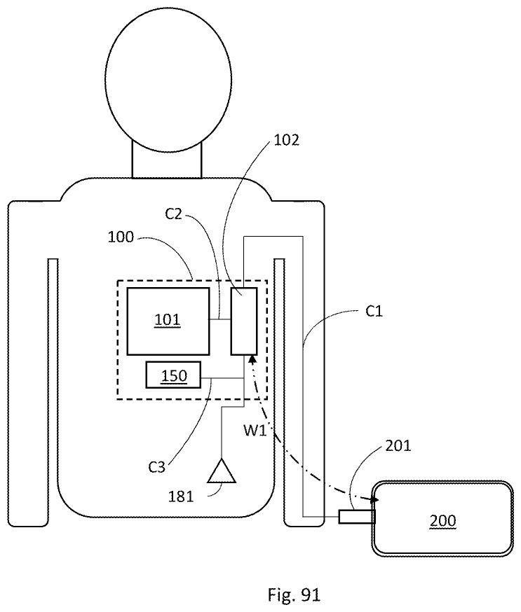

In aspect 257SE, increased security for communication between an external device 200 and an implant 100 is provided. FIGS. 90-93 shows embodiments of this aspect.

FIGS. 90A-C, 91 and 92 show an implant 100 implanted in a patient and an external device 200. The figures further show a sensation generator 181. The sensation generator 181 may be configured to generate a sensation. The sensation generator 181 may be contained within the implant 100 or be a separate unit. The sensation generator 181 may be implanted. The sensation generator 181 may also be located so that it is not implanted as such but still is in connection with a patient so that only the patient may experience sensations generated.

The implant 100 may be adapted for connection with the external device 200 and connected to a sensation generator 181 external to the implant 100. The implant 100 may be configured for receiving authentication data related to a sensation generated by the sensation generator 181 from the sensation generator 181. The implant 100 may be configured for storing the authentication data. The authentication data may be stored by a memory 107 of a communication unit 102 of the implant 100. The implant 100 may be configured to receive input authentication data from the external device 200. The implant 100 may further comprise an internal computing unit 106. The internal computing unit 106 may be configured for comparing the authentication data to the input authentication data and performing authentication of the connection between the implant 100 and the external device 200 based on the comparison.

The implant 100 may be configured for communicating further data to the external device 200 following positive authentication. The further data may be encoded as described herein under the second, third and sixth aspect. In these cases, the implant 100 and/or external device(s) comprises the necessary features and functionality (described in the respective sections of this document) for performing such encoding.

The sensation generator 181 may be adapted to generate a sensation detectable by a sense of the patient. The sensation generator 181 may be configured to, upon request, generate the sensation and transmit authentication data, related to the generated sensation, to the implant 100, implanted in the patient.

The sensation generator 181 may be configured to transmit the authentication data to the implant 100 using a wireless communication or connection. The implant 100 may for this reason comprise a wireless transceiver 108, configured for receiving the authentication data from the sensation generator 181 The sensation generator 181 may be configured to transmit the authentication data to the implant 100 using a wired or conductive communication or connection. The implant 100 may for this action comprise a wired transceiver 103, configured for receiving the authentication data from the sensation generator 181. The sensation generator 181 may further be configured to receive the request from the implant 100.

The sensation generator 181 may be configured to receive the request from the external device 200. The sensation generator 181 may be configured to receive the request from other external devices than the external device 200 such as for example a second external device being controlled by a health care provider of the patient.

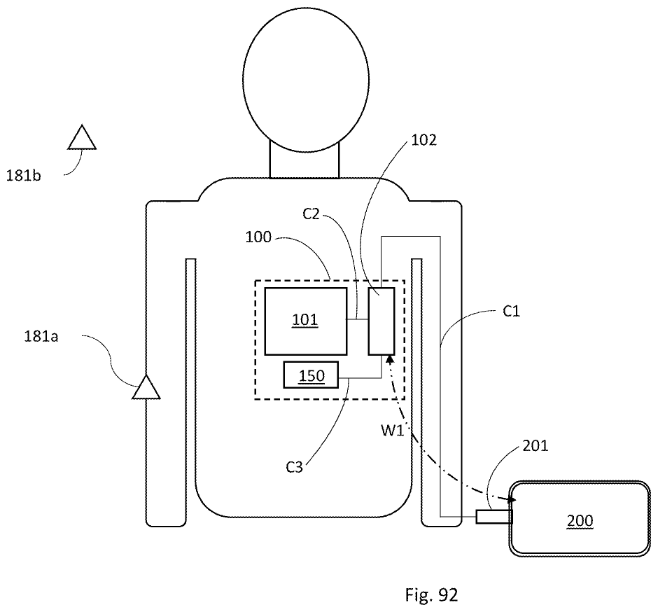

The sensation generator 181 may be adapted to be implanted in the patient. FIG. 92 shows a sensation generator 181a being configured to be worn in contact with the skin of the patient. This may e.g. be realized by having the sensation generator 181a being woven into or formed as a part of a clothing item of the patient. The sensation generator 181a may also be part of or worn as a bracelet of the patient or using any other suitable means of attachment.

FIG. 92 also shows a sensation generator 181b being configured to generate the sensation without being in physical contact with the patient. This may e.g. be realized by having a sensation generator 181b, normally only accessible to the patient, generating a visual sensation by a device not being implanted in the patient.

The sensation generator 181 may be configured to create the sensation comprising a plurality of sensation components. The sensation or sensation components may be created by providing a vibration, a sound, a photonic signal, a light signal, an electric signal, or a heat signal.

The sensation generator 181 may comprise a mechanical element for providing the vibration. The sensation generator 181 may comprise a loudspeaker for providing the sound. The sensation generator 181 may comprise a photon source for providing the photonic signal. The sensation generator 181 may comprise a light source for providing the light signal. The sensation generator 181 may comprise a power (current/voltage) source for providing the electric signal. The sensation generator 181 may comprise a thermal element for providing the heat signal. The heat signal may also be referred to as a thermal signal.

The mechanical element may be an electric, pneumatic, hydraulic, or thermodynamic motor or actuator. The loudspeaker may be adapted to provide sound of frequencies in the whole audible range from 20 Hz to 20 kHz. The loudspeaker may more preferably be adapted to provide low frequency sound in the range 20 Hz to 2 kHz.

The photon source and the light source may comprise light-emitting diodes (LEDs). The LEDs may be adapted to emit photons in the visible wavelength range from 380 nm to 750 nm. The LEDs may be adapted to emit photons in the infrared (IR) wavelength range 700 nm to 1 mm, but preferably in the near-infrared (NIR) wavelength range from 750 nm to 1400 nm. Note that visible wavelength range and IR wavelength range may overlap. The heat signal may be provided or generated by a photon source or a light source emitting in the IR wavelength range. The electric signal may be an electric signal in the form of a pulse or pulses.

The location of the sensation generator 181 may be configured to best fit the chosen sensory function of the patient and mitigate adverse physiological side effects as sensation power or strength may not need be as large. E.g. a mechanical sensation generator such as a vibration generator may be positioned with respect to the body of the patient and in particular mechanical sensory systems of the skin of the patient such that sensations i.e. vibrations need not be excessively powerful and cause the patient to feel discomfort, pain or other adverse effects as a result of the vibration. The sensation generator may also, due to this, be more discrete in its operation and with what type of sensation it generates, further increasing the security of the authentication as malicious third parties will find it harder to notice or find out what means of authentication an implant may require. An implanted sensation generator increases its discretion and thus the authentication security with it being effectively hidden within or by the body of the patient. A malicious third party, aiming to harm or gather medical information about the patient in which the implant is implanted by accessing the implant, may therefore need to perform a medical operation on the patient in question just to acquire information about how the implant may be accessed, essentially making the endeavor pointless, in the sense of hacking or accessing the implant remotely.

The sensation generator 181, the implant 100 and the external device 200 may be configured as a system for performing the methods.

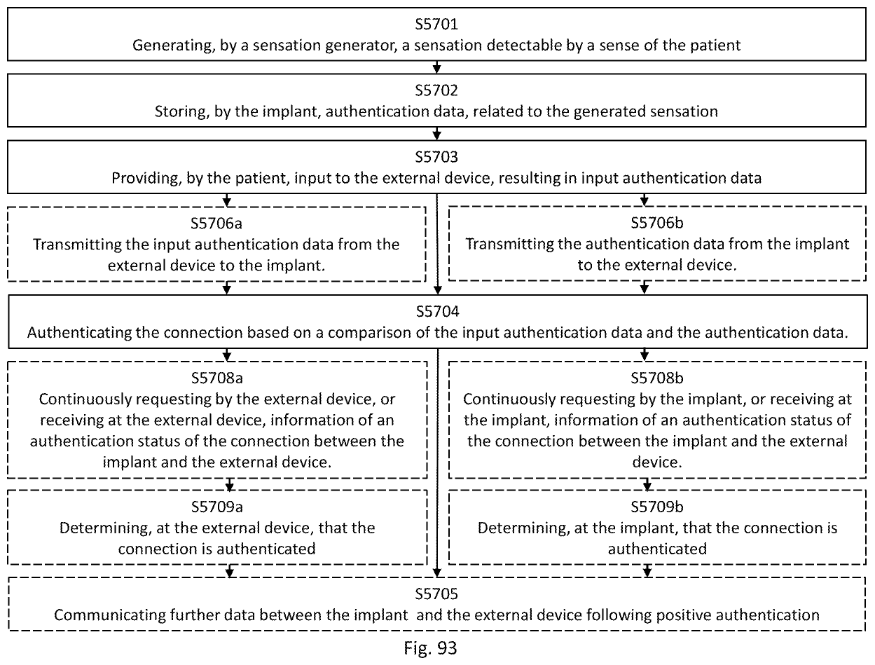

FIG. 93 shows a flow chart for methods of authenticating a connection between an implant 100 implanted in a patient and an external device 200. The method may comprise the step of generating S5701, by a sensation generator 181, a sensation detectable by a sense of the patient. The sensation may comprise a plurality of sensation components. The sensation or sensation components may comprise a vibration, a sound, a photonic signal, a light signal, an electric signal, or a heat signal.

The method may further comprise storing S5702, by the implant 100, authentication data, related to the generated sensation.

The method may further comprise providing S5703, by the patient, input to the external device 200, resulting in input authentication data. The method may further comprise authenticating S5704 the connection based on a comparison of the input authentication data and the authentication data. The authentication S5704 may be performed by either the implant 100 or the external device 200 which is further described below.

The authentication data may be communicated from the sensation generator 181 to the implant 100 using a wired communication. The authentication data may be communicated from the sensation generator 181 to the implant 100 using a wireless communication.

The step of authenticating S5704 the connection may comprise calculating a time difference between the timestamp of the sensation and the timestamp of the input from the patient, and upon determining that the time difference is less than a threshold, authenticating the connection.

The authentication data may comprise a timestamp of the sensation. The input authentication data may comprise a timestamp of the input from the patient. Authentication of the connection between the implant 100 and the external device 200 may comprise calculating a time difference between the timestamp of the sensation and the timestamp of the input from the patient, and upon determining that the time difference is less than a threshold, authenticating the connection. For example, if a sensation is generated by the sensation generator 181 with a timestamp referring to a time X and the user provides the input with a timestamp referring to a time Y, the difference between Y and X (Y-X) should be less than the threshold value T for authentication of the connection to occur. An example of a threshold value T may be Is. The comparison may also comprise a low threshold as to filter away input from the patient that is faster than normal human response times. The low threshold may e.g. be 50 ms. The patient input should be rejected if it is created before the actual sensation.

Patient inputs may require a waiting period after each input before the next one may be input. Continuous excessive inputs may cause the implant 100 or external device 200 to enter a security/lockdown mode wherein an even more secure form of authentication is required for it to be unlocked. Vital functions of e.g. the implant 100 may still be performed. The threshold, the low threshold, and the waiting period may further improve the security of the authentication.

The authentication data may comprise a number of times that the sensation is generated by the sensation generator 181. The input authentication data may comprise an input from the patient relating to a number of times the patient detected the sensation. Authenticating the connection may in this case comprise: upon determining that the number of times of the authentication data and the input authentication data are equal, authenticating the connection. A process for authentication may comprise the sensation generator 181 producing a sensation or sensation components pertaining to a specific number. This could e.g. mean producing a sensation or sensation components as a sequence of pulses, chronologically spaced such that they are, easily sensed by the patient. For example, the sensation or the sensation components may be generated such that a period of sensation has a duration. The sensation period may be followed by a duration period of no sensation before a next sensation period may commence. The patient may count the number of periods of sensation detected and input this number to authenticate the connection by comparing the number input by the patient to a number of sensation periods generated by the sensation generator 181 and stored by the implant 100 or other devices adapted to receive the data number of periods generated by the sensation generator 181.

The number of periods generated may be a randomly generated number and may preferably be in a range from 1-20. More preferably, the range is 1-10 and most preferably the range is 1-5.

The duration of a sensation period may preferably be in the range from 0-5s. More preferably, the range is from 0.1-3s. Most preferably the range is from 0.5-1.5s. Several sequences of sensation periods may be used to increase the security of the authentication and avoid unauthorized access rewarded to a would-be accessor merely guessing the right number.

These embodiments add extra security to the authentication as a number of sensations generated may not be predicted or otherwise acquired by a malicious third party via means such as, e.g. patient journals. Authentication in these ways may comprise a sequence of several sub-authentications which may be performed for even greater security and a rapidly reduced chance of “lucky guessing” by a malicious third party as the number of sub-authentications increase. Randomized values for sensation duration or the number of sensations generated may further increase security.

As is shown in FIG. 93, the method of authenticating may further and optionally comprise, communicating S5705 further data between the implant 100 and the external device 200 following positive authentication. The communication may use a wireless connection W1 or a wired/conductive connection C1. Further data may be transmitted both ways, i.e. from the implant 100 to the external device 200 and vice versa. Further data may also be communicated between the implant 100 or external device 200 and other external devices. Further data may comprise data sensed by a sensor 150 connected to the implant. The sensor 150 may be further described herein under aspect 256SE. In these cases, the implant 100 and/or external device(s) comprises the necessary features and functionality (described in the respective sections of this document).

Communication between the implant 100 and the external device 200 may be a wireless communication, using a wireless connection W1 or a wired/conductive communication, using a wired/conductive connection C1. Communication may be performed both ways.

Authentication data may be transmitted S5706b from the implant 100 to the external device 200. The step of authenticating S5704 the connection based on a comparison may then be performed by the external device 200. In this case, the implant 100 may continuously request (for example every 10 second, every 20 second, every minute, every two minute, etc.), or receive S5708b, information of an authentication status of the connection between the implant 100 and the external device 200, and upon determining S5709a, at the implant 100, that the connection is authenticated, transmitting S5705 further data from the implant 100 to the external device 200.

Input authentication data may be transmitted S5706a from the external device 200 to the implant 100. The step of authenticating S5704 the connection based on a comparison may then be performed by the implant 100. In this case, the external device 200 may continuously request (for example every 10 second, every 20 second, every minute, every two minute, etc.), or receive S5708a, information of an authentication status of the connection between the implant 100 and the external device 200, and upon determining S5709a, at the external device 200, that the connection is authenticated, transmitting S5705 further data from the external device 200 to the implant 100.

The further data may comprise data for updating a control program 110 running in the implant 100. The control program 110 may be stored by the memory 107. Further data may comprise operation instructions for operating the implant 100.

The sensation generator 181, sensation, sensation components, authentication data, input authentication data, and further data may be further described herein under aspect 248SE. In these cases, the implant 100 and/or external device(s) comprises the necessary features and functionality (described in the respective sections of this document). Providing a specialized sensation generator 181 for the generation of sensations may be advantageous is it may be optimized for the most ideal sensation generation. This may be put in contrast with e.g. an active unit or a motor that provides sensations as an effect of its operation. A specialized sensation generator 181 may also be preferable as the dual use of a motor or active unit may reduce its longevity by the extra stress associated with the dual use. This may be exemplified by a motor using its battery charge quicker when also used for authentication. It may be easier to optimize a sensation generator 181 compared to e.g. a motor or active device which needs to not neglect its main physiological purpose.

The implant may comprise at least one of:
a pacemaker unit,
an external heart compression device,
an apparatus assisting the pump function of a heart of the patient,
an operable artificial heart valve,
an implantable drug delivery device,
a hydraulic, mechanic, and/or electric constriction implant,
an operable volume filling device,
an operable gastric band,
an operable implant for stretching the stomach wall of the patient,
an operable cosmetic implant,
an implant for adjusting or replacing any bone part of a body of the patient,
an implant replacing an organ of the patient or part of an organ of the patient or the function thereof,
a vascular treatment device, and
an implant adapted to move fluid inside the body of the patient.

### Aspect 258SE Device Synchronization Sensation—Authenticating a Connection Between an Implant and the External Device by Using Sensations—Embodiments of Aspect 258SE of the Disclosure

The detailed description of embodiments for aspect V is hereby incorporated by reference.

The characteristics of the sensation characteristics may refer to different parts of the same signal or two entirely different physical signals. Parts of the same signal may be confined within a set amount of time and/or spaced by another set amount of time from each other. Different physical signals may be understood as one characteristic being a light-based sensation and as another characteristic (e.g. the second) being a sound-based sensation.

### Aspect 307SE Communication Remote Control—Remote Wake Signal—Embodiments of Aspect 307SE of the Disclosure

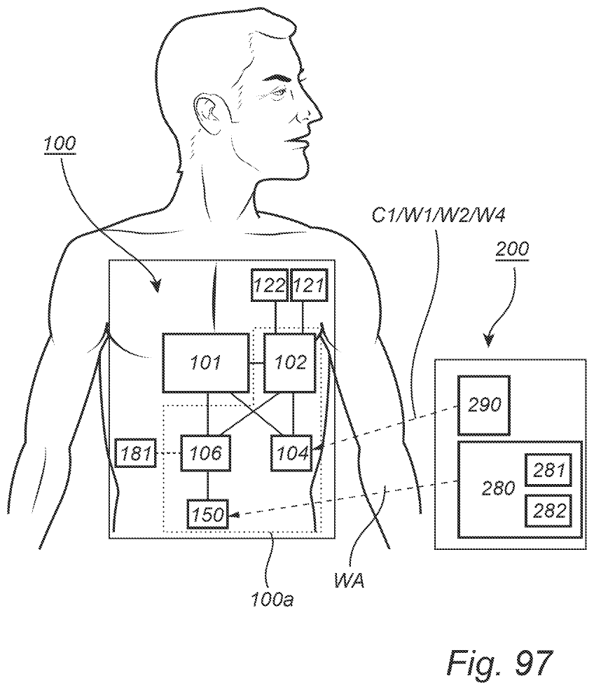

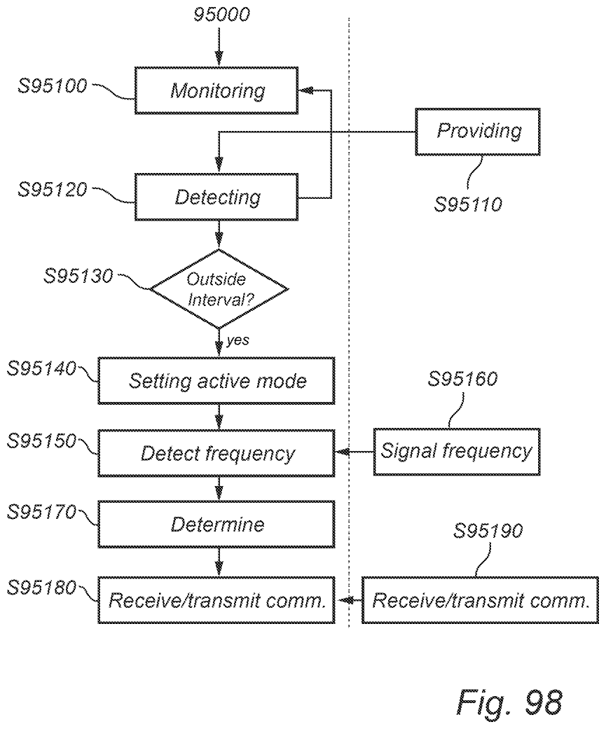

In aspect 307SE, a system and a method for communication between an external device 200 and an implant 100 is provided. FIGS. 97-98 shows embodiments of this aspect.

Generally, aspect 307SE defines a method, as shown in FIG. 98, adapted to run in a processor 106 comprised on an internal control unit 100a of an implant 100 when implanted in a patient, as shown in FIG. 97. The implant 100 and the external device 200 may be any of the implants 100 or external devices 200 described with reference to aspects one through twenty-one, further comprising the features described below.

FIG. 94 shows a system comprising an implant 100 and an external device 200. The implant comprises an internal control unit 100a for controlling a function of the implant. The internal control unit 100a comprises a processor 106 having a sleep mode and an active mode. The internal control unit 100a comprises a sensor 150 adapted to detect a magnetic field. The external device 200 comprises a signal provider 280 adapted to provide a magnetic field detectable by the internal sensor 150. The internal control unit 100a is further configured to, in response to a detected magnetic field exceeding a predetermined value, setting the processing unit 106 in an active mode.

By sleeping mode, it may be meant a mode with less battery consumption and/or processing power used in the processing unit 106, and by “active mode” it may be meant that the processing unit 106 is not restricted in its processing.

By an having a processing unit having a sleep mode and an active mode, the battery consumption of the processing unit may be decreased.

By having a sensor 150 adapted to detect a magnetic field and the internal control unit 100a adapted to set the processing unit 106 in the active mode in response to a detected signal, the external device 200 may cause a sleeping internal control unit 100a or processor 106 to “wake up”. In this way, the processing unit 106 may be set in the active mode when needed for communication with the external device 200.

In this example the processor 106 is shown separately from the communication unit 102, but in an alternative the processor may be comprised in the communication unit 102. The implant may comprise an implantable energy source 104, which in this example is shown as separate from the communication unit 102. As shown in FIG. 1, the implantable energy source may alternatively be comprised in the communication unit 102.

The sensor 150 may, for example, be a hall effect sensor, a fluxgate sensor, an ultra-sensitive magnetic field sensor, a magneto-resistive sensor, an AMR or GMR sensor, or the sensor may comprise a third coil having an iron core.

The magnetic field provider 280 comprised in the external device 200 may have an off state, wherein it does not provide any magnetic field, and an on state, wherein it provides a magnetic field. For example, the magnetic field provider 280 may comprise a magnet 281, a coil 281, a coil having a core 281, or a permanent magnet 281. In some embodiments, the magnetic field provider 280 may comprise a shielding means for preventing a magnet 281 or permanent magnet 281 from providing a magnetic field in the off state. In order to provide a substantially even magnetic field, the magnetic field provider may comprise a first and a second coil arranged perpendicular to each other.

After the processing unit 106 has been set in an active mode, i.e. when the processing unit 106 has been woken up, the implant may determine a frequency for further communication between the internal communication unit 102 and the external device 200. The implant 100 may thus comprise a frequency detector 121 for detecting a frequency for communication between the first 102 and the second communication units 290. The frequency detector 121 is, for example, an antenna. The external device 200 may comprise a frequency indicator 282, for transmitting a signal indicative of a frequency. The frequency indicator 282, may, for example, be a magnetic field provider capable of transmitting a magnetic field with a specific frequency. In some examples the frequency indicator is comprised in or the same as the magnetic field provider 281. In this way, the frequency signal is detected using means separate from the sensor, and can, for example, be detected using a separate pin or antenna comprised in the internal control unit 100a.

Alternatively, the internal communication unit 102 and the external device 200 may communicate using a predetermined frequency or a frequency detected by means defined by a predetermined method according to a predetermined protocol to be used for the communication between the internal communication unit 102 and the external device 200. The communication may comprise any of the protocols, authentication methods and/or encryption methods of aspects one through twenty-one described herein.

In some embodiments, the sensor 150 may be used for the communication. The communication may in these embodiments be performed with such that a frequency of the magnetic field generated by the coil is 9-315 kHz, or the magnetic field generated by the coil is less than or equal to 125 kHz, preferably less than 58 kHz. The frequency may be less than 50 Hz, preferably less than 20 Hz, more preferably less than 10 Hz, in order to be transmittable through a titan box.

In some embodiments, the internal control unit 100a comprises a receiver unit 122, and the internal control unit 100a and the external device 200 are configured to transmit and/or receive data via the receiver unit 122 via magnetic induction. The receiver unit 122 may comprise a high-sensitivity magnetic field detector, or the receiver unit may comprise a fourth coil for receiving the magnetic induction.

The system may implement a method 95000 for controlling a medical implant implanted in a patient as described with reference to FIG. 94. The method 95000 will now be described with reference to FIG. 95. The method 95000 comprises monitoring S95100 for signals by a sensor 150 comprised in an internal control unit 100a communicatively coupled to the active unit 101, providing S85110, from a signal provider 280 comprised in an external device 200, a wake signal, the external device 200 being adapted to be arranged outside of the patient's body, and setting S95120, by the internal control unit 100a and in response to a detected wake signal WS, a mode of a processing unit 106 comprised in the internal control unit from a sleep mode to an active mode.

The method 95000 may also comprise detecting S95120, using a frequency detector 121, a frequency for data communication between a first communication unit 102 and a second communication unit 290, the first communication unit 102 being associated with the internal control unit 100a and the second communication unit 290 being associated with the external device 200, wherein the frequency detector 121 is communicatively coupled to the internal control unit 100a or the external device 200. The detection may be performed using a detection sequence for detecting the frequency. This detection sequence may, for example, be a detection sequence defined in the protocol to be used for communication between the first and the second communication units. Potential protocols that may be used for communication between an internal communication unit 102 and an external device 200 has been described earlier in this description with reference to aspects one through twenty-two. The method 95000 may further comprise determining S95130, that the detected signal is above a predetermined threshold. Thus, the method 95000 may comprise determining S95170, using the frequency detector 121, the frequency for data communication, and initiating S95170, S95190 data communication between the first communication unit 102 and the second communication unit 290. The data communication can, for example, comprise one or more control instructions for controlling the medical implant 100 transmitted from the external device, or, for example, comprise data related to the operation of the medical implant 100 and be transmitted from the internal control unit 102. The second communication unit may comprise the features with figure references 201-270 which may comprise e.g. a wireless transceiver 208 and/or a computing unit 206.

### Aspect 308SE Energy Power-Supply Capacitor—Energy Burst Provider—Embodiments of Aspect 308SE of the Disclosure

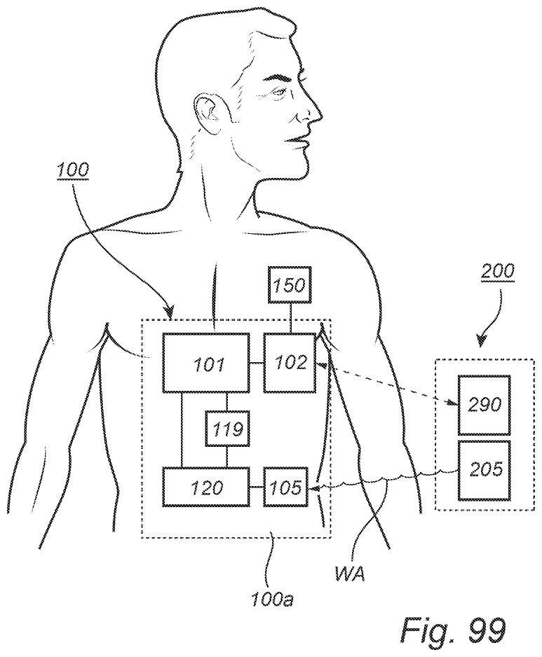

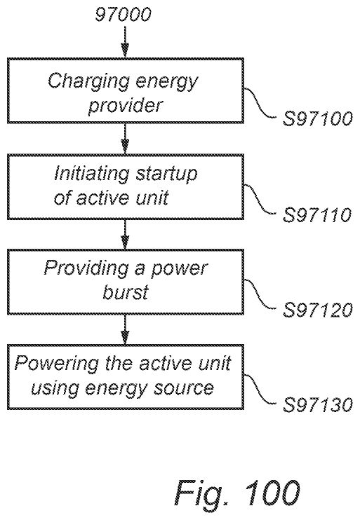

In aspect 308SE, an implant, and a method for powering an implant 100 is provided. FIGS. 99-100 shows embodiments of this aspect.

Generally, aspect 308SE defines an implant, as shown in FIG. 99, comprising an implant 100 comprising or be connected to a power supply for powering the implant 100. The implant 100, and the external device(s) 200 may comprise elements described above with reference to aspects one through twenty-two.

The power supply may comprise an implantable energy source 120 for providing energy to the implant 100 and/or the active unit 101, and an energy provider 119 connected to the implantable energy source 120 and connected to an energy consuming part 101 of the medical implant, the energy provider 119 being configured to store energy to provide a burst of energy to the energy consuming part 101, wherein the energy provider 119 is configured to be charged by the implantable energy source 120 and to provide the energy consuming part with electrical power during startup of the energy consuming part 101.

Alternatively, the implant 100 may comprise a first implantable energy source 120 for providing energy to an energy consuming part 101 of the medical implant 100, a second implantable energy source 119 connected to the implantable energy source 120 and connected to the energy consuming part, wherein the second implantable energy source 119 is configured to be charged by the implantable energy source 120 and to provide the energy consuming part 101 with electrical power during startup of the energy consuming part 101, wherein the second implantable energy source 119 has a higher energy density than the first implantable energy source 120. By having a “higher energy density” it may be meant that the second implantable energy source 119 has a higher maximum energy output per time unit than the first implantable energy source 120. The second energy storage 119 may be an energy provider as discussed below.

In this way, an energy consuming part 101 requiring a quick start or an energy consuming part which requires a high level or burst of energy for a start may be provided with sufficient energy. This may be beneficial as instead of having an idle energy consuming part 101 continuously using energy, the energy consuming part 101 may be completely turned off and quickly turned on when needed. Further, this may allow the use of an energy consuming part 101 needing a burst of energy for a startup while having a lower energy consumption when already in use. In this way, a battery or an energy source 120 having a slower discharging (or where a slower discharging is beneficial for the lifetime or health of the battery) may be used for the implant, as the extra energy needed for the startup may be provided by the energy provider.

Further, energy losses may occur in a battery or energy source of an implant if the battery or energy source is discharged too fast. These energy losses may for example be in the form of heat, which may damage the battery or energy source, or the body of the patient. By the implant described in these examples, energy may be provided from the battery or energy source in a way that does not damage the battery or energy source, which may improve the lifetime of the battery or energy source and thereby the lifetime of the medical implant.

The energy consuming part 101 may be any part of an implant requiring energy, such as a motor for operating a device or function of the medical implant, motor for powering a hydraulic pump, a restriction device, a stimulation device, a processing or computing unit, a communication unit, a device for providing electrical stimulation to a tissue portion of the body of the patient, a CPU for encrypting information, a transmitting and/or receiving unit for communication with an external unit (not shown as part of the energy consuming part 101 in the drawings, however, the communication unit 102 may be connected to the energy storage 120 and to the energy provider 119), a measurement unit or a sensor, a data collection unit, a solenoid, a piezo-electrical element, a memory metal unit, a vibrator, a part configured to operate a valve comprised in the medical implant, or a feedback unit.

In some examples, the discharging from the implantable energy source 120 during startup of the energy consuming part 101 is slower than the energy needed for startup of the energy consuming part 101, i.e. the implantable energy source 120 is configured to have a slower discharging than the energy needed for startup of the energy consuming part. That is, there may be a difference between the energy needed by the energy consuming part 101 and the energy the implantable energy source 120 is capable of providing without damaging the implantable energy source 120. In other words, a maximum energy consumption of the energy consuming part 101 may be higher than the maximum energy capable of being delivered by the implantable energy source 120 without causing damage to the implantable energy source, and the energy provider 119 may be adapted to deliver an energy burst corresponding to the difference between the required energy consumption and the maximum energy capable of being delivered by the implantable energy source 120. The implantable energy source 120 may be configured to store a substantially larger amount of energy than the energy burst provider 119 but may in some examples be slower to charge.

The implantable energy source 120 may be any type of energy source suitable for an implant 100, such as a re-chargeable battery or a solid-state battery, such as a trionychoid battery. The implantable energy source 120 may be connected to the energy consuming part 101 and configured to power the energy consuming part 101 after it has been started using the energy provider 119.

The energy provider 119 may be any type of part configured to provide a burst of energy for the energy consuming part 101. In some examples, the energy provider 119 is a capacitor, such as a start capacitor, a run capacitor, a dual run capacitor or a supercapacitor. The energy provider 119 may be connected to the implantable energy source 120 and be adapted to be charged using the implantable energy source 120. In some examples, the energy provider 119 may be a second energy provider 119 configured to be charged by the implantable energy source 120 and to provide the energy consuming part 101 with electrical energy.

A corresponding method 97000 for powering a medical implant will now be described with reference to FIG. 100. The method 97000 comprises the steps of initiating S97110 an energy consuming part 101 of the implant, the energy consuming part being connected to an implantable energy source 120, providing S97120 an initial burst of energy to the energy consuming part 101 using an energy provider 119 connected to the implantable energy source 120 and to the energy consuming part 101, the energy provider 119 being adapted to provide a burst of energy to the energy consuming part 101, and subsequently powering S97130 the energy consuming part 101 using the implantable energy source 120.

In some examples, a maximum energy consumption of the energy consuming part 101 is higher than the maximum energy capable of being delivered by the implantable energy source 120 without causing damage to the implantable energy source 120, and the energy provider 119 is adapted to deliver an energy burst corresponding to difference between the required energy consumption and the maximum energy capable of being delivered by the implantable energy source 120.

The method 97000 may further comprise the step of charging the energy provider 19 using the implantable energy source 120.

Initiating S97110 an energy consuming part 101 may comprise transitioning a control unit of the medical implant from a sleep mode to an operational or active mode.

The implantable energy source 120 may be adapted to be wirelessly charged and the implantable energy source may be connected to an internal charger 105 for receiving wireless energy from an external device 200 via an external charger 205, and the method 97000 may comprise wirelessly charging the implantable energy source 120. In some examples the method 97000 comprises controlling a receipt of electrical power from an external energy source at the internal charger 105. The internal energy 120 source may be charged via the receipt of a transmission of electrical power from an external energy source 205 by the internal charger 105.

### Aspect 309SE eHealth Broadcasting Data—Broadcasting Sensor Data from Implant—Embodiments of Aspect 309SE of the Disclosure

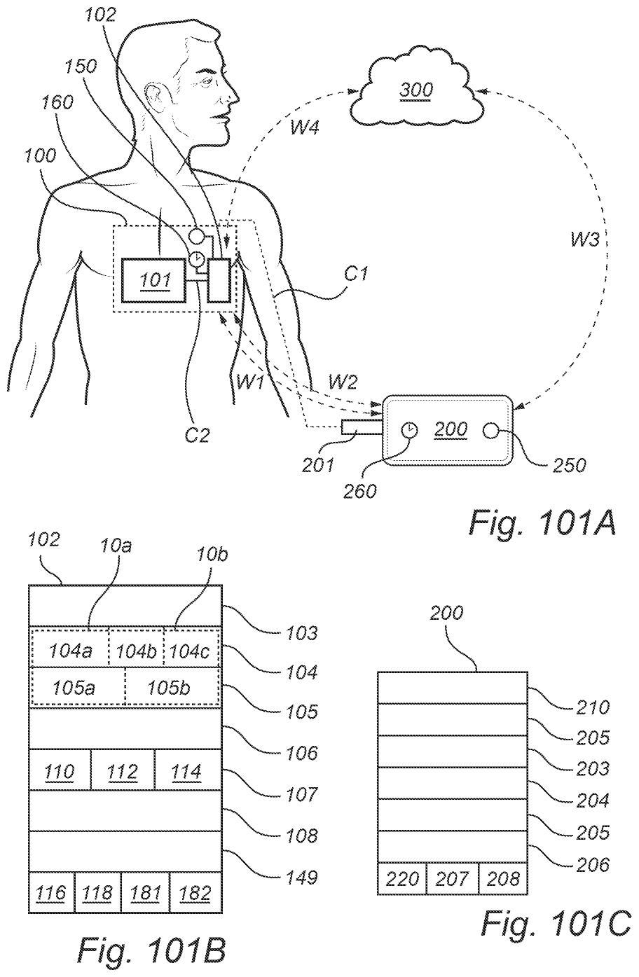

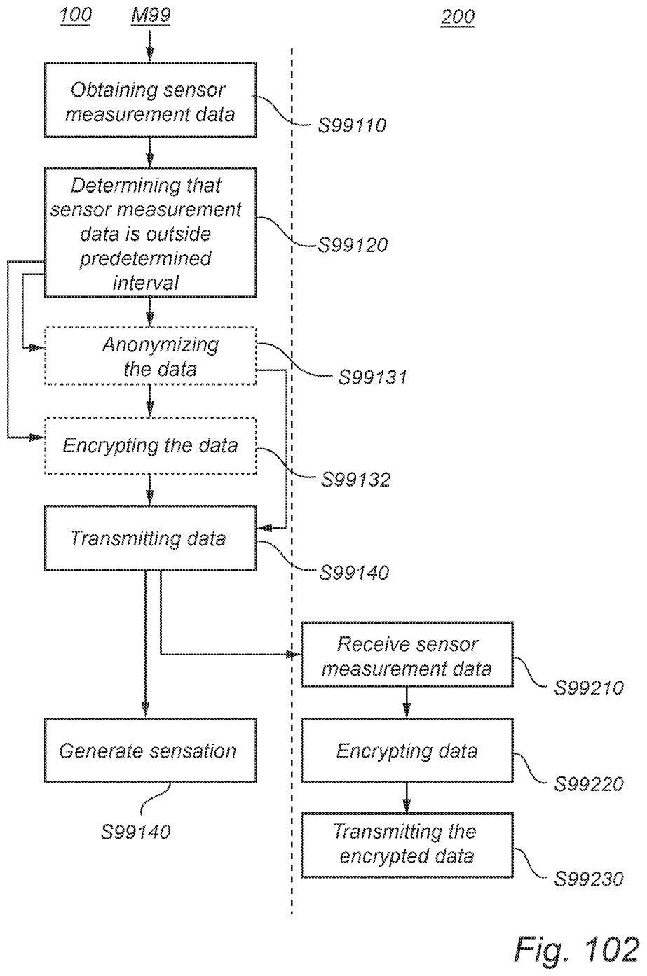

In aspect 309SE, a system and a method for communication between an external device 200 and an implant 100 is provided. FIGS. 101A-C and 102 shows embodiments of this aspect.

Generally, aspect 309SE defines a system, as shown in FIG. 101A, adapted to run in a processor 106 comprised in an internal control unit 100a of an implant 100 when implanted in a patient, as shown in FIG. 101A. The implant 100 and the external device 200 may be any of the implants 100 or external devices 200 described with reference to aspects one through twenty-three, further comprising the features described below.

According to the twenty fourth aspect, the system comprises at least one sensor 150 connected to the implant or comprised in the implant, for sensing at least one physiological parameter of the patient or a functional parameter of the implant. The sensor 150 is configured to periodically sense the parameter and the communication unit 102 is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant.

By broadcasting or transmitting information relating to the sensed parameter in response to a sensed parameter differing from a predetermined threshold or interval, an external device may detect that the implant is not functioning as expected, malfunctioning or not having the intended effect in the patient, or that a function of the patient's body is not functioning as expected. This malfunction or unexpected effect may be detected by the sensor measurements, and thus be transmitted to the external device. In this way, the implant may automatically, without any request from the external device, transmit data indicative of a malfunction or unexpected event, thus allowing for a safer device.

The transmission of data must however not be related to a malfunction or unexpected event but can be part of the normal workings of the implant. For example, the predetermined threshold or interval may be an expected threshold or interval, such as a pressure at a sphincter of a patient or in an organ of a patient, where the implant may be an artificial sphincter adapted to release as pressure of the sphincter in order to restore the pressure to a level within the predetermined interval. In this way, the external device may receive data indicative of the sensor measurements being outside of a predetermined interval or differing from a predetermined threshold, so that the user can, via the external device, perform any necessary adjustments or actions using the implant. This further allows for a safer implant.

The sensor 150 may, for example, be a pressure sensor, an electrical sensor, a clock, a temperature sensor, a motion sensor, an optical sensor, a sonic sensor, an ultrasonic sensor. The predetermined threshold or interval may depend on the sensor, and can for example be a predetermined interval for a pressure (such as a pressure at a sphincter or an organ of a patient, or a pressure at a hydraulic reservoir of the implant), a predetermined temperature interval or threshold (such as a temperature of the patient, or a temperature of a processing unit, a control unit, a power supply, or another part of the implant).

The internal communication unit 102 may be configured to broadcast or transmit the information using a short- to mid-range transmitting protocol, such as a Radio Frequency type protocol, a RFID type protocol, a WLAN type protocol, a Bluetooth type protocol, a BLE type protocol, a NFC type protocol, a 3G/4G/5G type protocol, or a GSM type protocol.

The control unit 100a of the implant may be connected to the sensor 150 and to the communication unit 102, and the control unit 100a may be configured to anonymize the information before it is transmitted. The transmission of data may comprise broadcasting of data.

In addition to, or as an alternative to, transmitting the data when the sensed parameter is differing form a predetermined threshold or interval, the communication unit 102 may be configured to broadcast the information periodically. The control unit 100a may be configured to cause the communication unit 102 to broadcast the information in response to a second parameter being above a predetermined threshold. The second parameter may, for example, be related to the control unit 100a itself, such as a free memory or free storage space parameter, or a battery status parameter. When the implant comprises an implantable energy source and an energy source indicator, the energy source indicator is configured to indicate a functional status of the implantable energy source and the indication may be comprised in the transmitted data. The functional status may indicate at least one of charge level and temperature of the implantable energy source.

In some embodiments the external device 200 is configured to receive the broadcasted information, encrypt the received information using an encryption key and transmit the encrypted received information. In this way, the external device 200 may add an additional layer of encryption or exchange the encryption performed by the internal communication unit. The encryption may be performed using any of the methods or systems described with reference to aspects one through nine.

In an embodiment, the internal communication unit 102 is configured to transmit the data using the body of the patient as a conductor C1, and the external device 200 is configured to receive the data via the body. Alternatively, or in combination, the communication unit of the implant is configured to transmit the data wirelessly to the external device W2.

Further, a method 99000 for transmitting data from an implant comprising a processor 106 and a communication unit 102, will now be described with reference to FIG. 99, comprising: obtaining S99110 sensor measurement data via a sensor 150 connected to or comprised in the implant 100, the sensor measurement relating to at least one physiological parameter of the patient or a functional parameter of the implant 100, and transmitting S99140 by the communication unit 102 the sensor measurement data in response to the sensor measurement differing from a predetermined threshold or being outside of a predetermined interval S99120, wherein the sensor 150 is configured to periodically sense the parameter. The method may further comprise broadcasting S99140 the sensor measurement data, to be received S 99210 by an external device 200. The transmitting or broadcasting may comprise using at least one of a Radio Frequency type protocol, RFID type protocol, WLAN type protocol, Bluetooth type protocol, BLE type protocol, NFC type protocol, 3G/4G/5G type protocol, or a GSM type protocol.

The method 99000 may further comprise, at the processor 106, anonymizing S99131, by the processor, the sensor measurement data before it is transmitted, or encrypting S99132 the sensor measurement data, using an encryptor 182 comprised in the processing unit, before it is transmitted. The encryption may be performed using any step(s) from the method described with reference to aspects one through nine described herein.

The transmitting S99140 of the data may further comprise to encode the data before the transmitting. The type of encoding may be dependent on the communication channel or the protocol used for the transmission.

The transmitting S99140 may be performed periodically, or in response to a signal received by the processor, for example, by an internal part of the implant such as a sensor 150, or by an external device 200.

The parameter may, for example, be at least one of a functional parameter of the implant (such as a battery parameter, a free memory parameter, a temperature, a pressure, an error count, a status of any of the control programs, or any other functional parameter mentioned in this description) or a parameter relating to the patient (such as a temperature, a blood pressure, or any other parameter mentioned in this description). In an example, the implant 100 comprises an implantable energy source 104 and an energy source indicator 104c, and the energy source indicator 104c is configured to indicate a functional status of the implantable energy source 104, and the sensor measurement comprises data related to the energy source indicator.

In one example, the transmitting S99140 comprises transmitting the sensor measurement to an internal processor 106 configured to cause a sensation generator 181 to cause a sensation S99150 detectable by the patient in which the implant 100 is implanted.

The method 99000 may be implemented in a system comprising the implant 100 and an external device 200, and further comprise receiving the sensor measurement data at the external device 200, and, at the external device 200, encrypting the sensor measurement data using a key to obtain encrypted data, and, transmitting the encrypted data. The transmitting may, for example, be performed wirelessly W3 or conductively C1.

### Aspect 310SE eHealth Double Encryption—Double Encryption—Embodiments of Aspect 310SE of the Disclosure

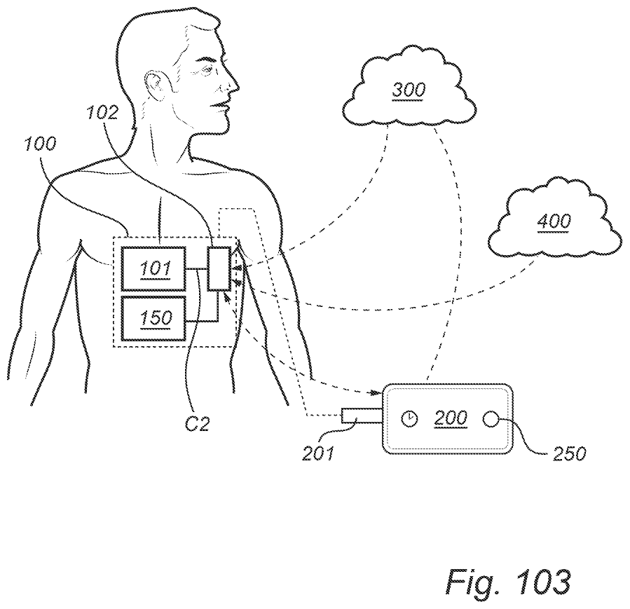

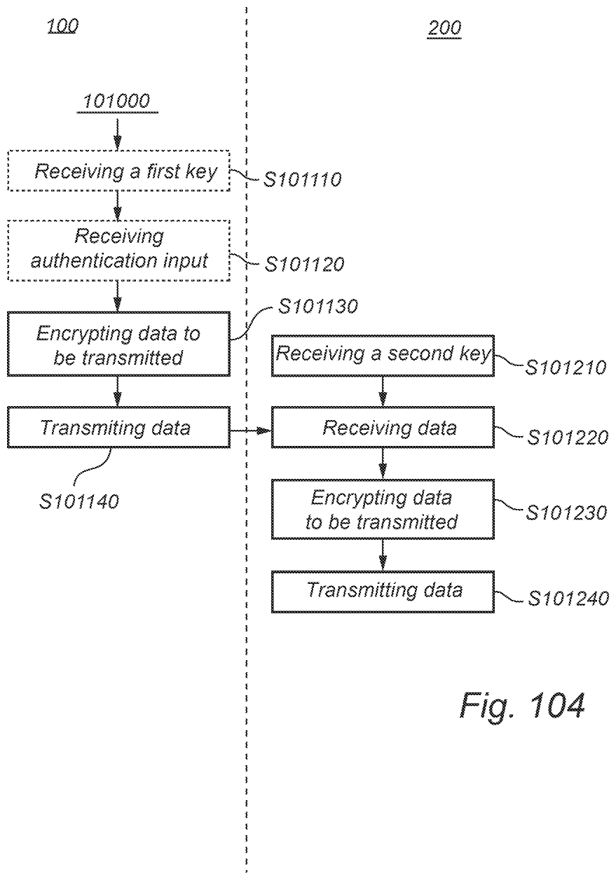

In aspect 310SE, a system and a method for communication between an external device 200 and an implant 100 is provided. FIGS. 103-104 shows embodiments of this aspect.

Generally, aspect 310SE defines a method, as shown in FIG. 104, adapted to run in a communication unit 102 comprised in an implant 100 when implanted in a patient, as shown in FIG. 103A. The implant 100 and the external device 200 may be any of the implants 100 or external devices 200 described with reference to aspects one through twenty-four, further comprising the features described below.

According to the system of aspect 310SE, a system is provided. The system comprises an implant 100 having a communication unit 102 configured to transmit data from the body of the patient to an external device 200, and an encryption unit 182 for encrypting the data to be transmitted. The system further comprises an external device 200 configured to receive the data transmitted by the communication unit 102, encrypt the received data using a first key and transmit the encrypted received data to a third external device 400. The encryption can be performed using any of the keys described above or below. In some embodiments, the external device 200 is configured to decrypt the data received from the internal communication unit 102 before encrypting and transmitting the data.

Thus, the implant 100 may transmit data to an external device 200 which may add an additional layer of encryption and transmit the data to a second external device 300. By having the external device add an additional layer of encryption, less computing resources may be needed in the implant, as the implant may transmit unencrypted data or data encrypted using a less secure or less computing resource requiring encryption. In this way, data can still be relatively securely transmitted to a third device. The transmission of data can be performed using any of the method described herein in addition to the method or in the system described below.

In one embodiment, the external device 200 may encrypt and transmit the data received from the internal communication unit 102 without decrypting it first.

In one example, the encryption unit 182 is configured to encrypt the data to be transmitted using a second key. The first key or the second key may, for example, implant specific information, a secret key associated with the external device, an identifier of the implant or an identifier of the communication unit 102. The second key could be a key transmitted by the external device 200 to the internal communication unit 102. In some examples, the second key is a combined key comprising a third key received by the implant from the external device 200.

The first key may be a combined key comprising a fourth key, wherein the fourth key is received by the external device 200 from a fourth device. The fourth device may be a verification unit, either comprised in the external device, or external to the external device and connected to it. The verification unit may have a sensor 250 for verification, such as a fingerprint sensor. More details in regard to this will be described below. Alternatively, the verification unit may be a generator, as described above.

The encryption performed by the internal 100 and/or external device 200 may be performed using any of the methods described with reference to aspect one through nine.

The system may be configured to perform authentication of the connection between the implant and the external device before transmitting of the data. The authentication may be performed using a sensed parameter, as described in aspect five. The implant 100 may in thus embodiment comprise a first sensor 150 for measuring the parameter of the patient at the implant 100. The external device 200 may comprise an external sensor 250 for measuring the parameter of the patient at the external device 200. The system may alternatively be configured to perform any of the authentication methods described in aspects one through four.

Further, a method for improving the security of data transmitted from an implant is provided. The method will now be described with reference to FIG. 104. The method 101000, for encrypted communication between an implant 100, when implanted in a patient's body, and an external device 200, comprises encoding or encrypting S101130, by the implant 100 or a processor 106 comprised in or connected to the implant 100, data relating to the implant 100 or the operation thereof; transmitting S101140, by a first communication unit 102 comprised in the implant 100, the data; receiving S101220, by a second communication unit comprised the external device 200, the data; encrypting S101230, by the external device 200, the data using an encryption key to obtain encrypted data; and transmitting S101340 the encrypted data to a third external device 400.

In this way, the external device 200 may add or exchange the encryption, or add an extra layer of encryption, to the data transmitted by the implant 100. When the implant encodes the data to be transmitted it may be configured to not encrypt the data before transmitting, or only using a light weight encryption, thus not needing as much processing power as if the implant were to fully encrypt the data before the transmission. By having an external device further encrypt the data, an improved security may be achieved while using relatively little processing power at the implant.

The encrypting S101130, by the implant 100, may comprise encrypting the data using a second key. The encryption using the second key may be a more light-weight encryption than the encryption performed by the external device using the second key, i.e. an encryption that does not require as much computing resources as the encryption performed by the external device 200.

The first or the second key may comprise a private key exchanged as described above with reference to encryption and authentication, or the first or the second key may comprise an implant specific information, a secret key associated with the external device, an identifier of the implant 100 or an identifier of the communication unit 102. They may be combined keys as described in this description, and the content of the keys, any combination of keys, and the exchange of a key or keys is described in the encryption and/or authentication of the general definition of features section. Thus, the method may comprise receiving S101110 a first key to be used for the encryption S101130 of the data, and/or receive 101120 authentication input for authenticating the connection with the external device in which the data will be transmitted. The external device may receive S101210 a second key to be used for the encryption S101230 of the data to be transmitted.

### Aspect 331SE Security Module

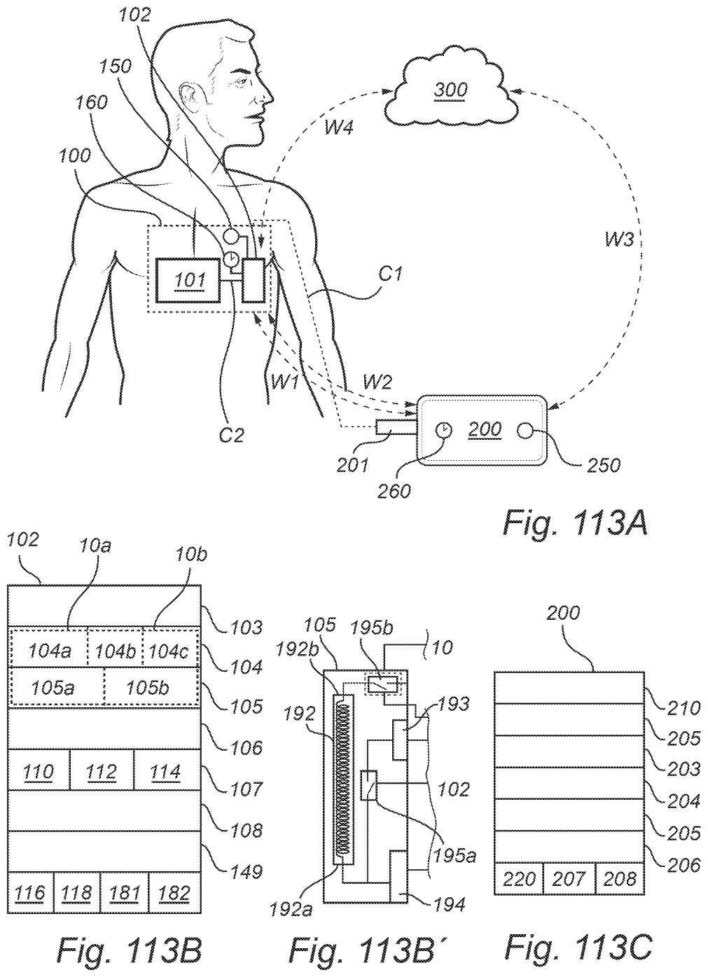

According to one embodiment described with reference to FIG. 113A-113C, the communication unit 102 or internal controller 102 or control unit 102 comprises a wireless transceiver 108 for communicating wirelessly with an external device, a security module 189, and a central unit, also referred to herein as a computing unit 106, which is to be considered as equivalent. The central unit 106 is configured to be in communication with the wireless transceiver 108, the security module 189 and the implantable medical device or active unit 101. The wireless transceiver 108 is configured to receive communication from the external device 200 including at least one instruction to the implantable medical device 100 and transmit the received communication to the central unit or computing unit 106. The central unit or computing unit 106 is configured to send secure communication to the security module 189, derived from the received communication from the external device 200, and the security module 189 is configured to decrypt at least a portion of the secure communication and verify the authenticity of the secure communication. In one embodiment, the security module is further configured to transmit a response communication to the central unit or computing unit 106 and the central unit or computing unit is configured to communicate the at least one instruction to the active unit 101. In another embodiment, the security module is configured to communicate the at least one instruction to the active unit 101 directly. In the embodiment shown in FIG. 113A-113C, the at least one instruction is based on the response communication, or a combination of the response communication and the received communication from the external device 200.

In the embodiment shown in FIG. 113A-113C, the security module 189 comprises a set of rules for accepting communication from the central unit or computing unit 106. In the embodiment shown in FIG. 113A-113C, the wireless transceiver 108 is configured to be able to be placed in an off-mode, in which no wireless communication can be transmitted or received by the wireless transceiver 108. The set of rules comprises a rule stipulating that communication from the central unit or computing unit 106 to the security module 189 or to the active unit 101 is only accepted when the wireless transceiver 108 is placed in the off-mode.

In the embodiment shown in FIG. 113A-113C, the set of rules comprises a rule stipulating that communication from the central unit or computing unit 106 is only accepted when the wireless transceiver 108 has been placed in the off-mode for a specific time period.

In the embodiment shown in FIG. 113A-113C, the central unit or computing unit 106 is configured to verify a digital signature of the received communication from the external device 200. The digital signature could be a hash-based digital signature which could be based on a biometric signature from the patient or a medical professional. The set of rules further comprises a rule stipulating that communication from the central unit 106 is only accepted when the digital signature of the received communication has been verified by the central unit 106. The verification could for example comprise the step of comparing the digital signature or a portion of the digital signature with a previously verified digital signature stored in the central unit 106. The central unit 106 may be configured to verify the size of the received communication from the external device and the set of rules could comprise a rule stipulating that communication from the central unit 106 is only accepted when the size of the received communication has been verified by the central unit 106. The central unit could thus have a rule stipulating that communication above or below a specified size range is to be rejected.

In the embodiment shown in FIG. 113A-113C, the wireless transceiver is configured to receive a message from the external device 200 being encrypted with at least a first and second layer of encryption. The central unit 106 the decrypts the first layer of decryption and transmit at least a portion of the message comprising the second layer of encryption to the security model 189. The security module 189 then decrypts the second layer of encryption and transmits a response communication to the central unit 106 based on the portion of the message decrypted by the security module 189 or transmits the decrypted data or a corresponding command to the active unit 101.

In the embodiment shown in FIG. 113A-113C, the central unit 106 is configured to decrypt a portion of the message comprising a digital signature, such that the digital signature can be verified by the central unit 106, also the central unit 106 is configured to decrypt a portion of the message comprising message size information, such that the message size can be verified by the central unit 106.

In the embodiment shown in FIG. 113A-113C, the central unit 106 is configured to decrypt a first and second portion of the message, and the first portion comprises a checksum for verifying the authenticity of the second portion.

In the embodiment shown in FIG. 113A-113C, the response communication transmitted from the security module 189 comprises a checksum, and the central unit 106 is configured to verify the authenticity of at least a portion of the message decrypted by the central unit 106 using the received checksum, i.e. by adding portions of the message decrypted by the central unit 106 and comparing the sum to the checksum.

In the embodiment shown in FIG. 113A-113C, the set of rules further comprise a rule related to the rate of data transfer between the central unit 106 and the security module 189. The rule could stipulate that the communication should be rejected or aborted if the rate of data transfer exceeds a set maximum rate of data transfer, which may make it harder for unauthorized persons to inject malicious code or instructions to the medical implant.

In the embodiment shown in FIG. 113A-113C, the security module 189 is configured to decrypt a portion of the message comprising the digital signature being encrypted with the second layer of encryption, such that the digital signature can be verified by the security module 189. The security module 189 then transmits a response communication to the central unit 106 based on the outcome of the verification, which can be used by the central unit 106 for further decryption of the message or for determining if instructions in the message should be communicated to the active unit 101. Alternatively, the security module determines at least one instruction for the active unit 302 based on the message, and transmits the message to the active unit 203 directly.

In the embodiment shown in FIG. 113A-113C, the central unit 106 is only capable of decrypting a portion of the received communication from the external device 200 when the wireless transceiver 108 is placed in the off-mode. In the alternative, or as an additional layer of security, the central unit 106 may be limited such that the central unit 106 is only capable of communicating instructions to the active unit 101 of the implantable medical device 100 when the wireless transceiver 108 is placed in the off-mode. This ensures that no attacks can take place while the central unit 106 is communicating with the active unit 101.

In the embodiment shown in FIG. 113A-113C, the implantable controller 102 is configured to receive, using the wireless transceiver 108, a message from the external device 200 comprising a first un-encrypted portion and a second encrypted portion. The implantable controller 102 (e.g. the central unit 106 or the security module 189) then decrypts the encrypted portion, and uses the decrypted portion to verify the authenticity of the un-encrypted portion. As such, computing power and thereby energy can be saved by not encrypting the entire communication, but rather only the portion required to authenticate the rest of the message (such as a checksum and/or a digital signature).

In the embodiment shown in FIG. 113A-113C, the central unit 106 is configured to transmit an encrypted portion to the security module 189 and receive a response communication from the security module 189 based on information contained in the encrypted portion being decrypted by the security module. The central unit 106 is then configured to use the response communication to verify the authenticity of the un-encrypted portion. The un-encrypted portion could comprise at least a portion of the at least one instruction to the implantable medical device 106. Alternatively, the central unit 306 is configured to transmit an encrypted portion and an unencrypted portion to the security module 389 and the security module 398 decrypts the encrypted portion and, using the decrypted portion, verifies the authenticity of the un-encrypted portion.

In the embodiment shown in FIG. 113A-113C, the implantable controller 102 is configured to receive, using the wireless transceiver 108, a message from the external device 200 comprising information related to at least one of: a physiological parameter of the patient and a physical parameter of the implanted medical device 100, and use the received information to verify the authenticity of the message. The physiological parameter of the patient could be a parameter such as a parameter based on one or more of: a temperature, a heart rate and a saturation value.

The physical parameter of the implanted medical device 100 could comprise at least one of a current setting or value of the implanted medical device 100, a prior instruction sent to the implanted medical device 100 or an ID of the implanted medical device 100.

The portion of the message comprising the information related to the physiological parameter of the patient and/or physical or functional parameter of the implanted medical device 100 could be encrypted, and the central unit 106 may be configured to transmit the encrypted portion to the security module 189 and receive a response communication from the security module 189 based on the information having been decrypted by the security module 189.

In the embodiment shown in FIG. 113A-113C, the security module 189 is a hardware security module comprising at least one hardware-based key. The security module 189 may have features that provide tamper evidence such as visible signs of tampering or logging and alerting. It may also be so that the security module 189 is “tamper resistant”, which makes the security module 189 inoperable in the event that tampering is detected. For example, the response to tampering could include deleting keys is tampering is detected. The security module 189 could comprise one or more secure crypto processor chip. The hardware-based key(s) in the security module 189 could have a corresponding hardware-based key placeable in the external device 200. The corresponding external hardware-based key could be placed on a key-card connectable to the external device 200.

In one embodiment, the security module 387 and the central unit 309 are both comprised in a multi-processor, wherein the security module 387 runs on a first processor and the central unit runs on second processor, different from the first.

In alternative embodiments, the security module 189 is a software security module comprising at least one software-based key, or a combination of a hardware and software-based security module and key. The software-based key may correspond to a software-based key in the external device 200. The software-based key may correspond to a software-based key on a key-card connectable to the external device 200.

In the embodiment shown in FIG. 113A-113C, the external device 200 is a handheld external device, however, in alternative embodiments, the external device may be a remote external device or a cloud based external device In the embodiment shown in FIG. 113A-113C, the at least one instruction to the implantable medical device 100 comprises an instruction for changing an operational state of the implantable medical device 100.

In the embodiment shown in FIG. 113A-113C, the wireless transceiver 108 is configured to communicate wirelessly with the external 200 device using electromagnetic waves at a frequency below 100 kHz, or more specifically below 40 kHz. The wireless transceiver 108 is thus configured to communicate with the external device 200 using “Very Low Frequency” communication (VLF). VLF signals have the ability to penetrate a titanium housing of the implantable medical device 100, such that the electronics of the implantable medical device 100 can be completely encapsulated in a titanium housing.

The wireless transceiver 108 is configured to communicate wirelessly with the external device 200 using a first communication protocol and the central unit 106 is configured to communicate with the security module 189 using a second, different, communication protocol. This adds an additional layer of security as security structures could be built into the electronics and/or software in the central unit 106 enabling the transfer from a first to a second communication protocol. The wireless transceiver 108 may be configured to communicate wirelessly with the external device using a standard network protocol, which could be one of an RFID type protocol, a WLAN type protocol, a Bluetooth (BT) type protocol, a BLE type protocol, an NFC type protocol, a 3G/4G/5G type protocol, and a GSM type protocol. In the alternative, or as a combination, the wireless transceiver 108 could be configured to communicate wirelessly with the external device 200 using a proprietary network protocol. The wireless transceiver 108 could comprises a Ultra-Wide Band (UWB) transceiver and the wireless communication between the implantable controller 102 and the external device 200 could thus be based on UWB. The use of UWB technology enables positioning of the remote control 320″ which can be used by the implanted medical device 100 as a way to establish that the external device 200 is at a position which the implanted medical device 100 and/or the patient can acknowledge as being correct, e.g. in the direct proximity to the medical device 100 and/or the patient, such as within reach of the patient and/or within 1 or 2 meters of the implanted medical device 100. In the alternative, a combination of UWB and BT could be used, in which case the UWB communication can be used to authenticate the BT communication, as it is easier to transfer large data sets using BT.

### Aspect 332SE—Variable Impedance

According to one embodiment described with reference to FIG. 113A-113C, the communication unit 102 or controller of the implantable medical device 100 comprises a receiving unit 105 or energy receiver 105 comprising a coil 192 (specifically shown in FIG. 113B′) configured for receiving transcutaneously transferred energy. The receiving unit further comprises a measurement unit 194 configured to measure a parameter related to the energy received by the coil 192 and a variable impedance 193 electrically connected to the coil 192. The receiving unit 105 further comprises a switch 195a placed between the variable impedance 193 and the coil 192 for switching off the electrical connection between the variable impedance 193 and the coil 192. The communication unit 102 or controller 102 is configured to control the variable impedance 193 for varying the impedance and thereby tune the coil 192 based on the measured parameter. The communication unit 102 or controller 102 is further configured to control the switch 195a for switching off the electrical connection between the variable impedance 193 and the coil 192 in response to the measured parameter exceeding a threshold value. The controller 102 may further be configured to vary the variable impedance in response to the measured parameter exceeding a threshold value. As such, the coil can be tuned or turned off to reduce the amount of received energy if the amount of received energy becomes excessive. The measurement unit 194 is configured to measure a parameter related to the energy received by the coil 192 over a time period and/or measure a parameter related to a change in energy received by the coil 192 by for example measure the derivative of the received energy over time. The variable impedance 193 is in the embodiment shown in FIG. 113B′ placed in series with the coil 192. In alternative embodiments it is however conceivable that the variable impedance is placed parallel to the coil 192.

The first switch 195a is placed at a first end portion 192a of the coil 192, and the implantable medical device 100 further comprises a second switch 195b placed at a second end portion of the coil 192, such that the coil 192 can be completely disconnected from other portions of the implantable medical device 100. The receiving unit 105 is configured to receive transcutaneously transferred energy in pulses according to a pulse pattern. The measurement unit 194 is in the embodiment shown in FIG. 113B′ configured to measure a parameter related to the pulse pattern. The controller 102 is configured to control the variable impedance in response to the pulse pattern deviating from a predefined pulse pattern. The controller 102 is configured to control the switch 195a for switching off the electrical connection between the variable impedance 193 and the coil 192 in response to the pulse pattern deviating from a predefined pulse pattern. The measurement unit is configured to measure a temperature in the implantable medical device 100 or in the body of the patient, and the controller 102 is configured to control the first and second switch 195a, 195b in response to the measured temperature.

The variable impedance 193 may comprise a resistor and a capacitor and/or a resistor and an inductor and/or an inductor and a capacitor. The variable impedance 193 may comprise a digitally tuned capacitor or a digital potentiometer. The variable impedance 193 may comprise a variable inductor. The first and second switch comprises a semiconductor, such as a MOSFET. The variation of the impedance is configured to lower the active power that is received by the receiving unit. As can be seen in FIG. 113B′, the variable impedance 193, the first and second switch 195a, 195b and the measurement unit 194 are connected to the communication unit/controller 102 and the receiving unit 105 is connected to an energy storage unit 10 such that the energy storage unit 10 can store energy received by the receiving unit 105.

### Aspect 311SE eHealth Data Integrity—Verifying Data Integrity from/to Implant and from/to External Device—Embodiments of Aspect 311SE of the Disclosure

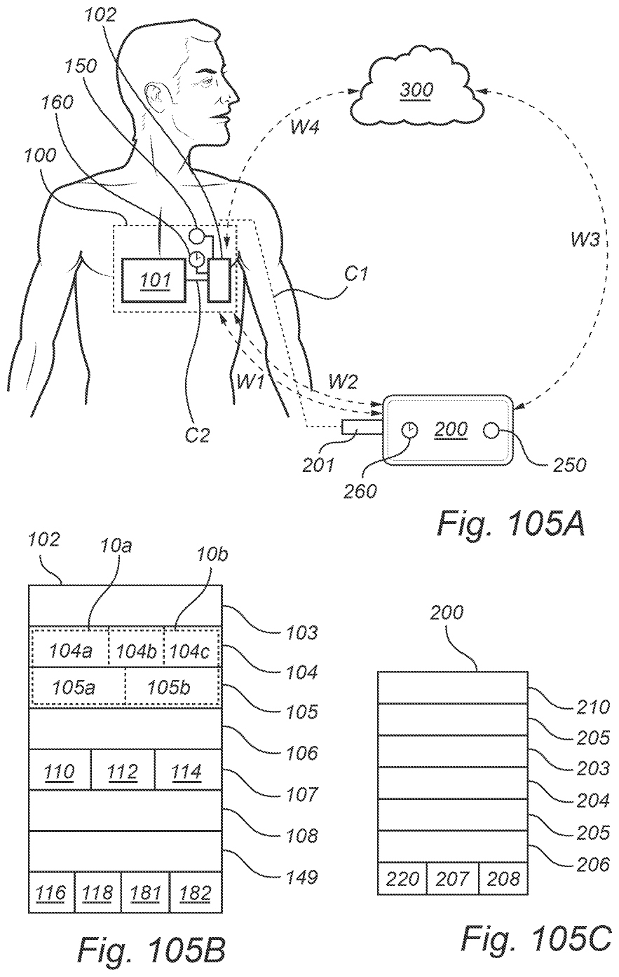

In aspect 311SE, a system and a method for communication between an external device 200 and an implant 100 is provided. FIGS. 105A-C and 106-107 show embodiments of this aspect.

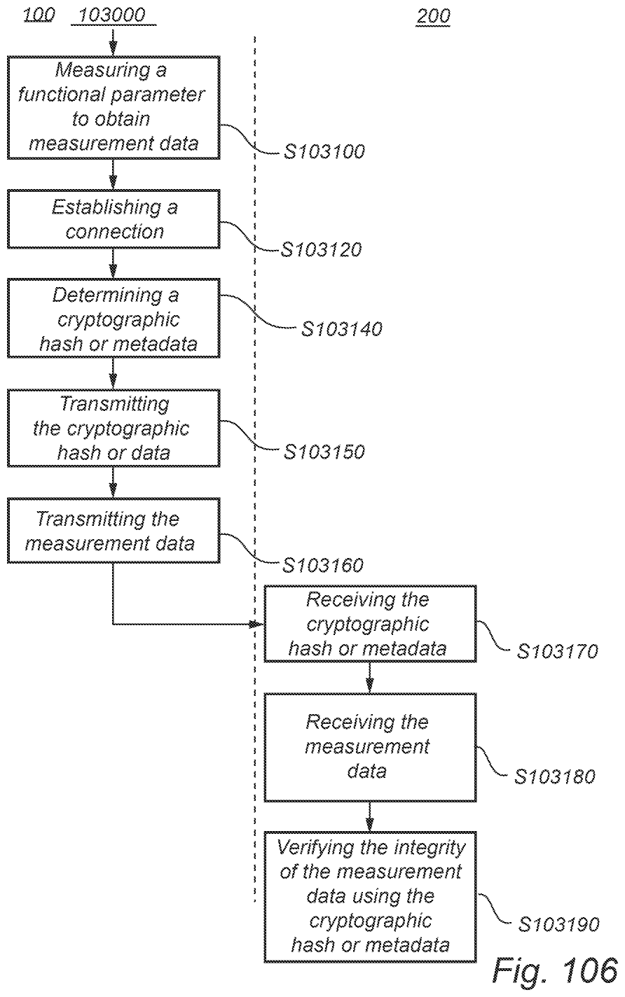

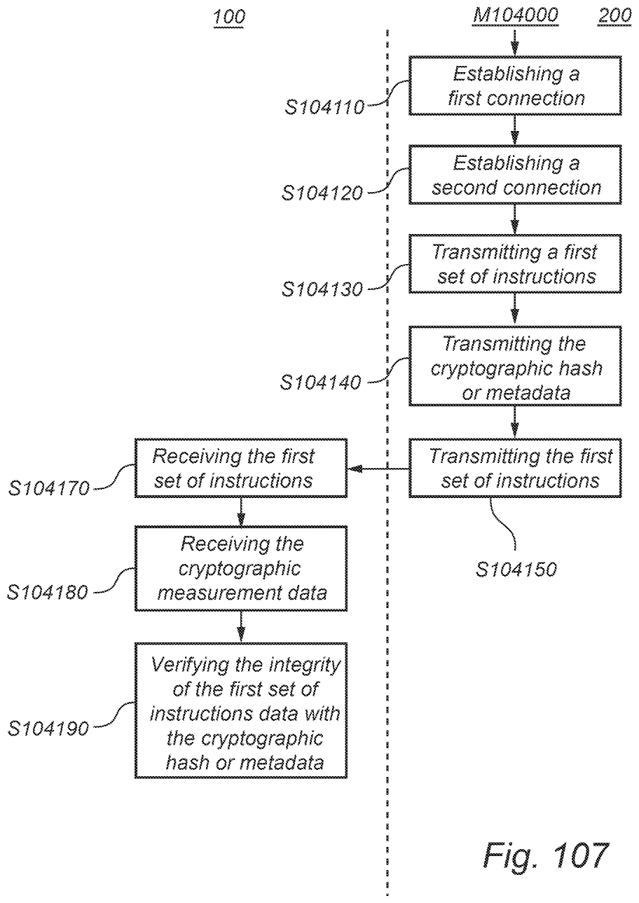

Generally, aspect 311 SE defines a method, as shown in FIG. 106, and a method, as shown in FIG. 107, both adapted to run in a communication unit 102 comprised in an implant 100 when implanted in a patient, as shown in FIGS. 105A-C. The implant 100 and the external device 200 may be any of the implants 100 or external devices 200 described with reference to aspects one through twenty-five, further comprising the features described below.

In the examples or embodiments transmitting data from or to the implant 100, the following method may be implanted in order to verify the integrity of the data, described with reference to FIGS. 105A-C. By verifying the integrity of the data, an external device 200 or a processor 106 comprised in the implant may verify that the data has not been corrupted or tampered with during the transmission. In some examples, data integrity for data communicated between an implant 100 and an external device 200 or between an external device 200 and an implant 100 may be performed using a cyclic redundancy check.

Thus, a system for transmitting data related to a parameter of the implant is provided and will not be described with reference to FIGS. 105A-C. The system comprises an implant 100 and an external device 200. The implant 100 comprises a processor 106, a sensor 150 for measuring the parameter, and an internal communication unit 102. The sensor 150 is configured to obtain measurement data related to the parameter, and the communication unit is configured to establish a connection between the internal communication unit 102 and the external device 200, the external device being configured to receive data from the implant. The processor 106 is further configured to determine a cryptographic hash or a metadata relating to the measurement data and adapted to be used by the external device 200 to verify the integrity of the received data. The processor 106 is further configured to transmit the cryptographic hash or metadata and to transmit the measurement data.

The parameter may, for example, be a parameter of the implant, such as a temperature, a pressure, a battery status indicator, a time period length, a pressure at a restriction device, a pressure at a sphincter, or a physiological parameter of the patient, such as a pulse, a blood pressure, or a temperature. In some examples, multiple parameters may be used. The sensor may, for example, be a pressure sensor, an electrical sensor, a clock, a temperature sensor, a motion sensor, an optical sensor, a sonic sensor, an ultrasonic sensor.

The processor 106 or the external device 200 may be further configured to evaluate the measurement data relating to the functional parameter. By evaluating it may be meant to determine if the parameter is exceeding or less than a predetermined value, differing from a predetermined interval, to extract another parameter from the measurement data, compare the another parameter to a predetermined value, or displaying the another parameter to a user. For example, the external device 200 may be configured to determine, based on the evaluating, that the implant 100 is functioning correctly, or determining based on the evaluating that the implant 100 is not functioning correctly.

If it is determined that the implant 100 is not functioning correctly, the external device 200 may be configured to transmit a corrective command to the implant 100. The corrective command may be received at the implant 100, and the implant may run the corrective command correcting the functioning of the implant 100 according to the corrective command. The corrective command or process referred to here could, for example, be the reset function described with reference to the aspect 244SE.

Thus, the external device 200 is configured to receive the transmitted cryptographic hash or metadata, receive the measurement data, and verifying the integrity of the measurement data using the cryptographic hash or metadata. The cryptographic hash algorithm be any type of hash algorithm, i.e. an algorithm comprising a one-way function configured to have an input data of any length as input and produce a fixed-length hash value. For example, the cryptographic hash algorithm may be MD5, SHA1, SHA 256, etc.

In some examples, the cryptographic hash is a signature obtained by using a private key of the implant, and wherein the verifying, by the external device 200, comprises verifying the signature using a public key corresponding to the private key.

When using a cryptographic hash, the external device may calculate a second cryptographic hash for the received measurement data using a same cryptographic hash algorithm as the processor, and determining that the measurement data has been correctly received based on that the cryptographic hash and the second cryptographic hash are equal (i.e. have the same value).

When using a metadata the verifying the integrity of the data may comprise obtaining a second metadata for the received measurement data relating to the functional parameter, and determining that the data has been correctly received based on that metadata and the second metadata are equal. The metadata may, for example, be a length of the data, or a timestamp, or other data for verifying the integrity of the received measurement data.

In some examples the measurement data is transmitted in a plurality of data packets. In those examples, the cryptographic hash or metadata comprises a plurality of cryptographic hashes or metadata each corresponding to a respective data packet, and the transmitting of each the cryptographic hashes or metadata is performed for each of the corresponding data packets.

A corresponding method for evaluating a functional parameter of an implant when implanted in a patient will also be described with reference to FIG. 106. The method 103000 comprises measuring S103100, using the sensor, the functional parameter to obtain measurement data, establishing S103120 a connection between the internal communication unit and an external device configured to receive data from the implant, determining S103140, by the processor, a cryptographic hash or a metadata relating to the measurement data and adapted to be used by the external device to verify the integrity of the received data, and transmitting S103150 the cryptographic hash or metadata, and transmitting S103160, from the communication unit, the measurement data.

The method 103000 may further comprise, at the external device, receiving S103170 the transmitted cryptographic hash or metadata, receiving S103180 the measurement data, and verifying S103190 the integrity of the measurement data with the cryptographic hash, metadata or information relating to the functional parameter. The verification may be performed as described above with reference to FIGS. 105A-C.

When the cryptographic hash or metadata comprises a cryptographic hash, the verifying S103190 the integrity of the measurement data may comprise calculating a second cryptographic hash for the received measurement data using a same cryptographic hash algorithm as the processor, and determining that the measurement data has been correctly received based on that the cryptographic hash and the second cryptographic hash are equal.

The cryptographic hash algorithm may comprise one of: MD5, SHA1, or SHA 256. The person skilled in the art would know several options for exchanging keys for implementing the cryptographic hash algorithms. Further, any exchange of keys described herein, for example with reference to any of aspects 1-10, may be used.

In some examples, the cryptographic hash is a signature obtained by using a private key of the implant, and wherein the verifying, by the external device, comprises verifying the signature using a public key corresponding to the private key.

When the cryptographic hash or metadata comprises a metadata, the verifying the integrity of the data may comprise obtaining a second metadata for the received measurement data relating to the functional parameter, and determining that the data has been correctly received based on that metadata and the second metadata are equal.

The metadata may be any data related to the measurement date, such as for example, a length of the data, a timestamp, or a sensor measurement. The sensor measurement may, for example, be used as described with reference to aspect eight.

The method may further comprise, at the external device, evaluating the measurement data relating to the functional parameter. The evaluating may be performed as described above with reference to FIGS. 105A-C. The method may further comprise, at the external device, determining, based on the evaluating, that the implant is functioning correctly. For example, if the parameter or measurement data is within a predetermined interval or less than or exceeding a predetermined threshold, the external device may determine that the implant is functioning correctly.

Correspondingly, the method may comprise determining that the implant is not functioning correctly of if the parameter or measurement data is not within a predetermined interval or not less than or exceeding a predetermined threshold sending. The method may in those examples further comprise, transmitting, from the external device, a corrective command to the implant, receiving the corrective command at the implant, and correcting the functioning of the implant according to the corrective command. The corrective command may, for example, be a command related to the active unit or a command related to the processor or communications unit. In some examples, the corrective command is a command to restart the processor, reset the processor or invoking a corrective signal for the active unit.

In some examples, the measurement data is transmitted in a plurality of data packets, wherein the cryptographic mash or metadata comprises a plurality of cryptographic hashes or metadata each corresponding to a respective data packet, and wherein the transmitting of each the cryptographic hashes or metadata is performed for each of the corresponding data packets.

In a specific embodiment of the method, the method is for evaluating a pressure at a sphincter of the patient.

A similar method may be utilized for communicating instructions from an external device 200 to an implant 100 implanted in a patient, the method will now be described with reference to FIG. 107. The method 104000 is configured to be performed, for example, in a system as described with reference to FIGS. 105A-C. The method 104000 comprises establishing S104110 a first connection between the external device 200 and the implant 100, establishing S104120 a second connection between a second external device 300 and the implant 100, transmitting S104130, from the external device 200, a first set of instructions to the implant 1200 over the first connection, transmitting S104140, from the second external device 300, a first cryptographic hash or metadata corresponding to the first set of instructions to the implant, and, at the implant 100, verifying S104180 the integrity of the first set of instructions and the first cryptographic hash or metadata, based on the first cryptographic hash or metadata. The external device 200 may be separate from the second external device 300.

The first connection may be established between the internal communication unit 102 and a transceiver of the external communication unit 201, 203. In some examples, the communication using the second connection is performed using a different protocol than a protocol used for communication using the first communication channel. In some examples, the first connection is a wireless connection and the second connection is an electrical connection. The second connection may, for example, be an electrical connection using the patient's body as a conductor. The protocols and ways of communicating may be any communication protocols described in this description with reference to C1, and W1-W8. The establishing of the first and second connections are performed according to the communication protocol used for each of the first and the second connections.

When using a cryptographic hash, the verifying S104180 the integrity of the first set of instructions may comprise calculating a second cryptographic hash for the received first set of instructions using a same cryptographic hash algorithm as the processor 106, and determining that the first set of instructions has been correctly received based on that the cryptographic hash and the second cryptographic hash are equal. The cryptographic hash may, for example, be a signature obtained by using a private key of the implant 100, and wherein the verifying comprises verifying the signature using a public key corresponding to the private key. In some examples, the cryptographic hash is a signature obtained by using a private key of the implant, and wherein the verifying comprises verifying the signature using a public key corresponding to the private key. The private keys and public keys, as well as the exchange or transmittal of keys have been described in this description. Alternatively, other well-known methods can be used for transmitting or exchanging a key or keys between the external device 200 and the implant 100.

When using a metadata, and wherein the verifying S104180 the integrity of the data may comprise obtaining a second metadata for the received first set of instructions, and determining that the first set of instructions has been correctly received based on that metadata and the second metadata are equal. The metadata may, for example, be any type of data relating to the data to be transmitted, in this example the first set of instructions. For example, the metadata may be a length of the data to be transmitted, a timestamp on which the data was transmitted or retrieved or obtained, a size, a number of packets, or a packet identifier.

In some examples, the implant 100 may transmit data to an external device 200 relating to the data information in order to verify that the received data is correct. The method 104000 may thus further comprise, transmitting, by the implant 100, information relating to the received first set of instructions, receiving, by the external device 200, the information, and verifying, by the external device 200, that the information corresponds to the first set of instructions sent by the external device 200. The information may, for example, comprise a length of the first set of instructions.

The method 104000 may further comprise, at the implant 100, verifying the authenticity of the first set of instructions by i. calculating a second cryptographic hash for the first set of instructions, ii. comparing the second cryptographic hash with the first cryptographic hash, iii. determining that the first set of instructions are authentic based on that the second cryptographic hash is equal to the first cryptographic hash, and upon verification of the authenticity of the first set of instructions, storing them at the implant.

In some examples, the first set of instructions comprises a cryptographic hash corresponding to a previous set of instruction, as described in other parts of this description.

In some examples, the first set of instructions may comprise a measurement relating to the patient of the body for authentication, as described in other parts of this description.

### Aspect 312SE eHealth Programming Predefined Steps—Programming Via Predefined Steps—Embodiments of Aspect 312SE of the Disclosure

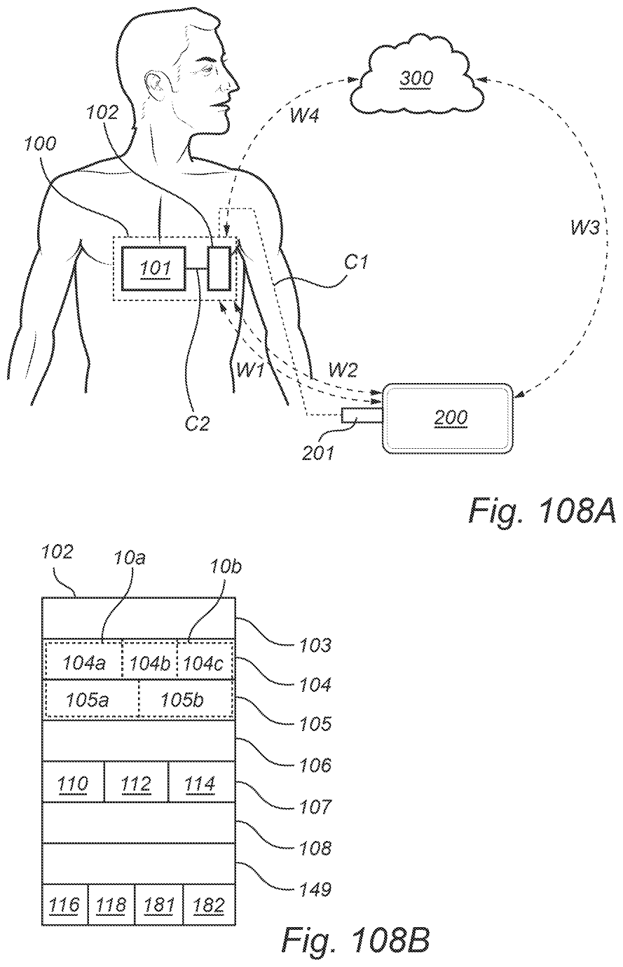

In aspect 312SE, a method for communication between an external device 200 and an implant 100 is provided. FIGS. 108A-B and 109 shows embodiments of this aspect.

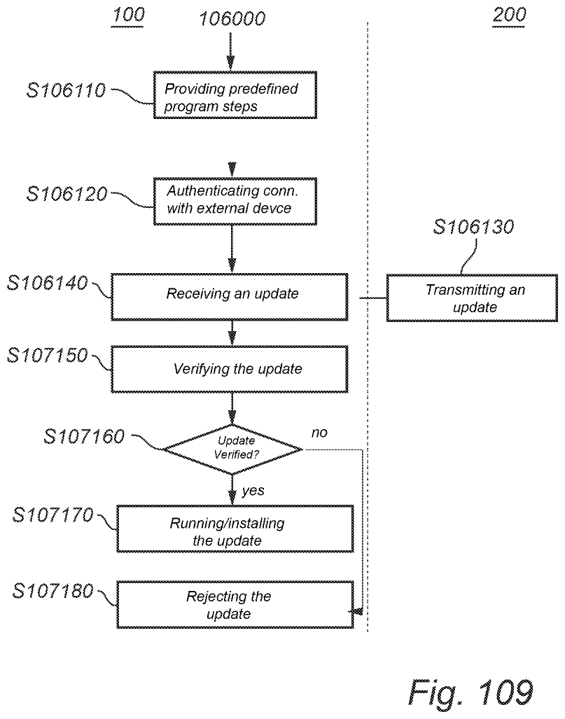

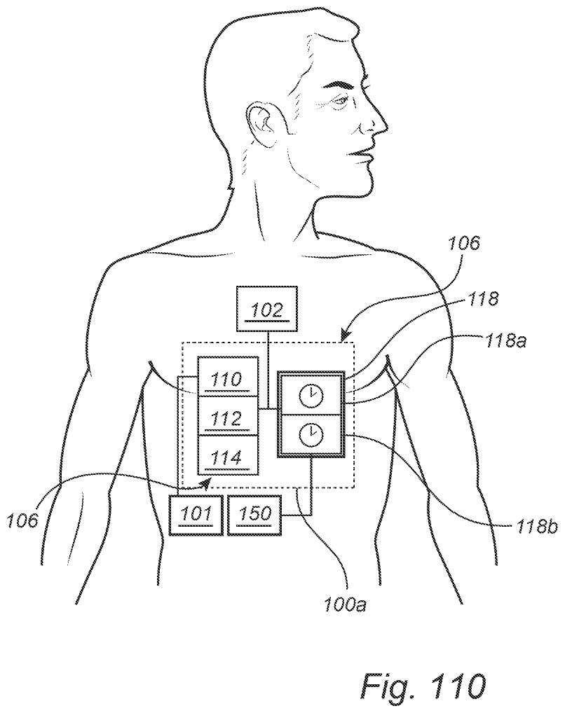

Generally, aspect 311 SE defines a method, as shown in FIG. 109, and a system, as shown in FIGS. 108A-B. The system comprises an implant 100 an external device, which may be any of the implants 100 or external devices 200 described with reference to aspects one through twenty-six, further comprising the features described below.

As described above with reference to the general definition of features and the aspect 244SE, the implant may comprise a control program of the implant. The control program may be any software used for controlling the implant, and may be updatable, configurable, or replaceable. A system for updating or configuring a control program of the implant is now described with reference to FIGS. 108A-C.

The implant 100 may comprise an internal computing unit 106 configured to control a function of said implant 100, the internal computing unit 106 comprises an internal memory configured to store: i. a first control program 110 for controlling the internal computing unit, and ii. a second, configurable or updatable, with predefined program steps, control program 112 for controlling said function of said implant 100, and iii. a set of predefined program steps for updating the second control program 112. The internal computing unit 106 may further comprise or be connected to an internal communication unit 102, the internal communication unit being configured to communicate with an external device 200, wherein said internal computing unit 106 is configured to receive an update to the second control program 112 via said internal communication unit 102, and a verification function of, connected to, or transmitted to said internal computing unit 102, said verification function being configured to verify that the received update to the second control program 112 comprises program steps comprised in the set of predefined program steps. In this way, the updating or programming of the second control program may be performed using predefined program steps, which may decrease the risk that the new or updated control program is incorrect or comprises malicious software, such as a virus, spyware or a malware.

The predefined program steps may comprise setting a variable related to a pressure, a time, a minimum or maximum temperature, a current, a voltage, an intensity, a frequency, an amplitude of electrical stimulation, a feedback mode (sensorics or other), a post-operative mode or a normal mode, a catheter mode, a fibrotic tissue mode (for example semi-open), an time open after urination, a time open after urination before bed-time.

The verification function may be configured to reject the update in response to the update comprising program steps not comprised in the set of predefined program steps and/or be configured to allow the update in response to the update only comprising program steps comprised in the set of predefined program steps.

The internal computing unit may be configured to install the update in response to a positive verification, for example by a user using an external device, by a button or similarly pressed by a user, or by another external signal.

The authentication or verification of communications between the implant and an external device has been described above with reference to any of aspects one to eleven.

A corresponding method for programming an implant by an external device will now be described with reference to FIG. 109. The implant comprises an internal computing unit configured to control a function of said implant and an internal memory configured to store: a first control program for controlling the internal computing unit, a second, updatable or configurable, control program for controlling said function of said implant, and a set of predefined program steps for updating the second control program, the external device being configured to communicate with the implant via a first connection. The method comprises providing S106110, at the internal computing unit, a set of predefined program steps for updating the second control program; transmitting S106130, by the external device, an update comprising a subset of the predefined program steps over the first connection; receiving S106140, at the internal computing unit, the update, verifying S106150, by the internal computing unit, that the update comprise a subset of the predefined program steps, and upon verification S106160 of the instructions, running S106170 the update at the implant.

By verifying that an update to a control program for the implant is comprised in a set of predetermined program steps, the security may be improved. For example, this may lower the risk of malicious instructions being run at the implant, as such instructions would probably not be part of the predetermined program steps. Further, this decreases the risk that someone updating the control program would provide an update with an error that would be dangerous to the patient.

The predefined steps may, for example, comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.

The verifying S107150 may comprise rejecting S106180 the update in response to the update comprising program steps not comprised in the set of predefined program steps. By rejecting it is meant that the update is not run and/or not installed at the implant.

The verifying S170150 may comprise the verifying comprise allowing S106170 the update in response to the update only comprising program steps comprised in the set of predefined program steps. By allowing it is meant that the update is run and/or installed at the implant. The method may further comprise, upon verification, installing the update.

In an embodiment, the method further comprises authenticating S106120, S106125 the communication between the implant and the external device over a second connection. The authentication may be performed using any of the authentication methods described herein.

In some embodiments, the second connection is a wireless short-range connection. Alternatively, the second connection is an electrical connection using the patient's body as a conductor. Both of these options have been described with reference to C1-C3 and W1-W8 in other aspects of this description.

The method may further comprise or be combined with any embodiment of aspect 307SE.

### Aspect 313SE eHealth Watchdog—Safety Reset Function—Embodiments of the Twenty-Eighth Aspect of the Disclosure

According to aspect 313SE a method and a system for improved safety of the implant is provided. The safety reset function will now be described with reference to FIG. 110. In addition to or as an alternative to the reset function 116 described above with reference to the general definition of features, the implant may comprise another reset function, herein called the first reset function.

The implant shown in FIG. 110 comprises an internal processor 106 or an internal computing unit 106 (comprising an internal processor) having a second control program 112 for controlling a function of the implant, and a safety reset function 118. The safety reset function 118 may comprise a first reset function 118a, and optionally a second reset function 118a and/or optionally a third reset function. In this example, the internal processor 106 and the safety reset function 118 are shown as separate from the communication unit 102 but may advantageously be comprised in the communication unit 102. The implant 100 and the external device 200 shown in FIG. 110 may further have any features or implement any method disclosed herein with reference to an implant 100 and an external device 200, in addition to the first reset function.

The first reset function 118a may be configured to restart or reset said second control program 112 in response to: i. a timer of the first reset function 118a has not been reset, or ii. a malfunction in the first control program.

The first reset function 118a may, for example, comprise a computer operating properly, COP, function connected to the internal computing unit 106.

The first reset function 118a may comprise a timer, and the first or the second control program 112 is configured to periodically reset the timer. If the time is not reset, i.e. if the timer times out, the first reset function 118a may be configured to request a status from the second control program 112, and in response to an absent or invalid response, restart or reset the second control program 112. Alternatively, in response to the timer timing out, the first reset function 118a may restart or reset the second control program 112.

The first reset function 118a, may alternatively, or in combination with the timer, be configured to monitor a status function of the second control program 112. In some examples, the first reset function 118a is configured to periodically request a status from a status function of the second control program 112, and in response to an invalid or absent response restart or reset the second control program 112.

The first reset function 118a may be configured to restart or reset the first or the second control program 112 using a second reset function 118b. In these embodiments, the first reset function 118a may be configured to reset a timer of the second reset function 118b in response to the timer of the first reset function 118a being reset, for example by the second control program 112. When the timer of the first reset function 118a times out, the first reset function 118a may be configured to send a corrective command to the second control program 112. If the corrective action is successful, the second control program 112 may be configured to reset the timer of the first reset function 118a, and the first reset function 118a may then reset the timer of the second reset function 118b. If the corrective action is not successful, the second control program 112 will not reset the timer of the first control program 118a, and the first control program 118a will thus not reset the timer of the second control program 118b. The timer of the second reset program 118b will thus eventually time out, and in response to the timer of the second reset program 118b timing out, the second reset program 118b may reset or restart the second control program 112. In this way, firstly, the first reset program 118a will try to correct the second control program 112, and in the correction is unsuccessful, secondly, the second reset function 118b will restart or reset the second control program 112. This may help avoid unnecessary restarts or resets.

In some examples, the reset or restart of the second control program 112 may be performed by invoking a reset function of the first control program 110, such as, for example, described above with reference to the reset function 116.

The safety function 118 may further comprise a third reset function connected to or comprised in the internal computing unit 106 and connected to the second reset function 118a. The third reset function may in an example be configured to trigger a corrective function for correcting the first 110 or second control program 112, and the second reset function is configured to restart the first 110 or second control program 112 sometime after the corrective function has been triggered. The corrective function may be a soft reset or a hard reset.

The second or third reset function may, for example, configured to invoke a hardware reset by triggering a hardware reset by activating an internal or external pulse generator which is configured to create a reset pulse. Alternatively, the second or third reset function may be implemented by software.

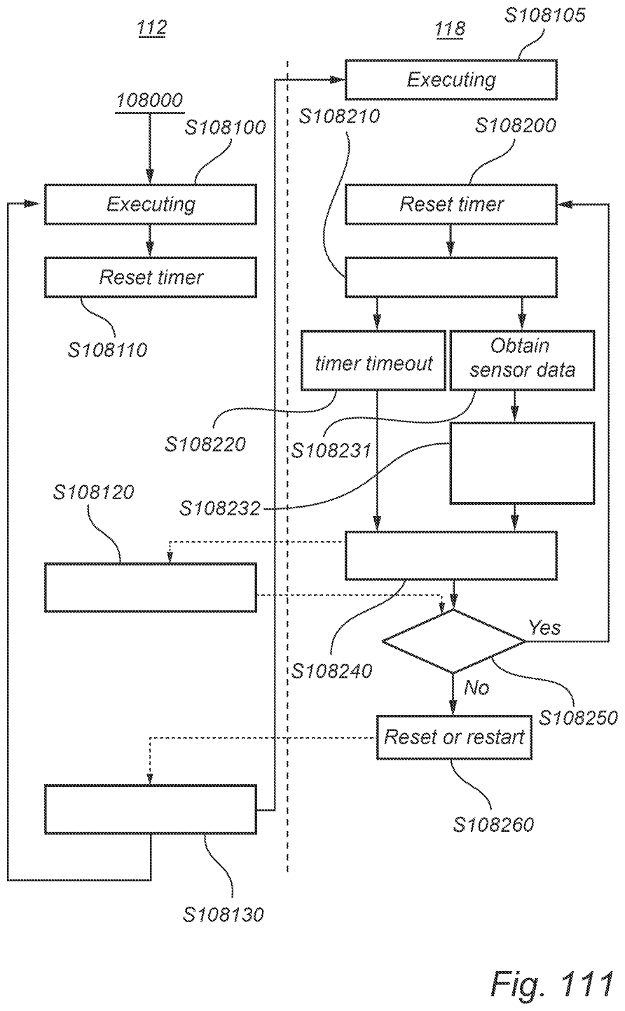

A corresponding method will now be described with reference to FIG. 111. The method 108000 for controlling a control program of an implant, when implanted in a patient, the implant comprising a processor for running the first control program, comprises: executing S108100 the first control program at the internal computing unit, executing S108105 a first reset function; resetting or restarting S108260 the first control program by the first reset function in response a detection S108250 of a malfunction in the first control program.

In some examples, the resetting or restarting of the first control program comprises triggering S108240 a corrective function for correcting the first control program.

The method 108100 may further comprise periodically resetting S108110, by the first control program, the first reset function, wherein the detecting S108210 of a malfunction comprises determining that the first reset function has not been reset for a predetermined period of time. In this example, the timer of the first control program may thus time out S108220.

Alternatively, or in combination, the method 108000 the detecting of a malfunction comprises obtaining S108231 data relating to the functioning of the first control program, for example by invoking a status function of the second control program or by obtaining measurement data relating to the functioning of the implant, detecting S108232 that a sensor measurement relating to a physiological parameter of the patient or a parameter of the implant being less than, exceeding or differing from a predetermined value. If it is determined that the obtained data indicates a malfunction, a corrective function S108120 of the second control program may be invoked S108240.

In some examples, the sensor measurement relates to a pressure in a part of the implant, to a pressure in a reservoir or a restriction device of the implant, a pressure in an organ of the patient's body.

For example, the physiological parameter of the patient or a parameter of the implant may be a temperature.

In some examples, the reset function comprises invoking a first control program comprising a safety measure. The safety measure may, for example, be to turn off a restrictive function of the implant, such as a restriction device at a sphincter of the patient.

The method 108100 may further comprise monitoring a function of the implant or the first control program, and wherein the reset function is configured to in response to an incorrect or absent response for the monitoring program, reset or restart the first control program.

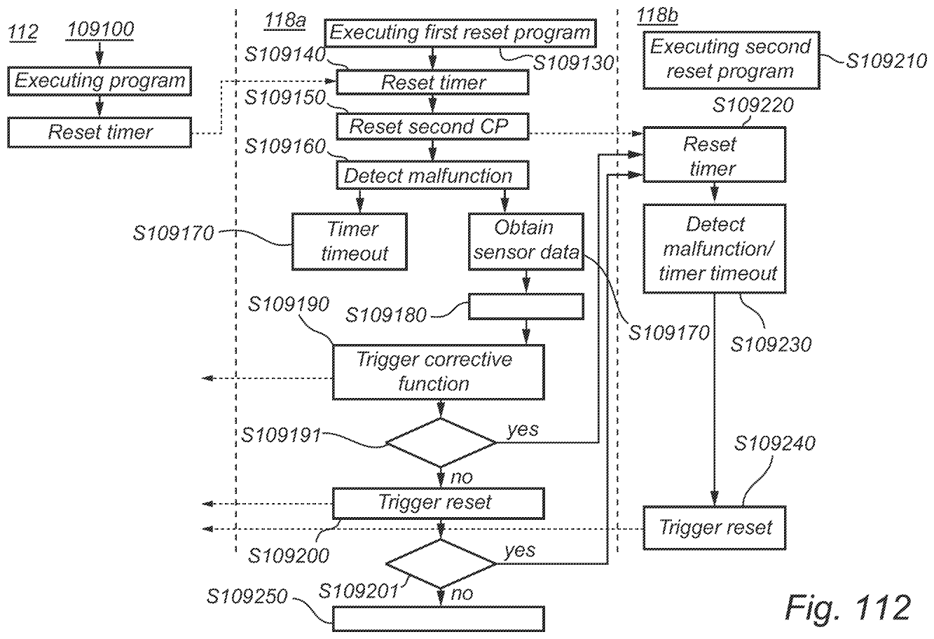

In some examples, the reset function 118 comprises, in addition to the first reset function 118a, a second reset function 118b. A method for a reset function having a first reset function and a second reset function will now be described with reference to FIG. 112.

The method comprises may, in relation to the first reset function, comprise the same steps as described above with reference to FIG. 111. The method may comprise executing S109130 a first reset program, resetting S109140 a timer of the first rest program by the second control program, and resetting S109150, by the first reset program, a timer of the second reset program, in response to the timer of the first reset program being reset.

The method may comprise detecting S109160 a malfunction in the second control program or in the implant, as described above with reference to FIG. 108, and in response to a detected malfunction, trigger S109190, by the first control program, a corrective function.

If the corrective function is successful, the second control program may reset S109120 the timer of the first reset program, and the first reset program may in turn reset the timer of the second reset program. Generally, the timer of the second reset function is longer that the timer for the first reset function.

If the corrective function is not successful, the first control program may be configured to trigger S109200 a reset function of the second control program S109200. This may, for example, be a soft (i.e. a software implemented) reset. If the reset function invocation is not successful, the first control program may wait S109250 for the timer of the second control program to detect S109230 that the malfunction is still present, and trigger S109240 a reset of the second control program 112. This reset may, for example, be a hard (i.e. a hardware implemented) reset.

The second reset program 118b may determine or detect the second control program 112 is malfunctioning by that the timer of the second reset program 118b has timed out or expired, or that the second control program 112 is malfunctioning, similarly to the detecting S109160 of the malfunction of the first reset program by the use of measurement data. In response to a detected malfunction the method may further comprise to trigger, by the second reset program 118a, a reset of the second control program 112.

The method may further comprise invoking a safety measure, wherein the safety measure comprises controlling a function of the implant.

### Aspect 314SE eHealth Logging—Update Confirmation—Embodiments of Aspect 314SE of the Disclosure

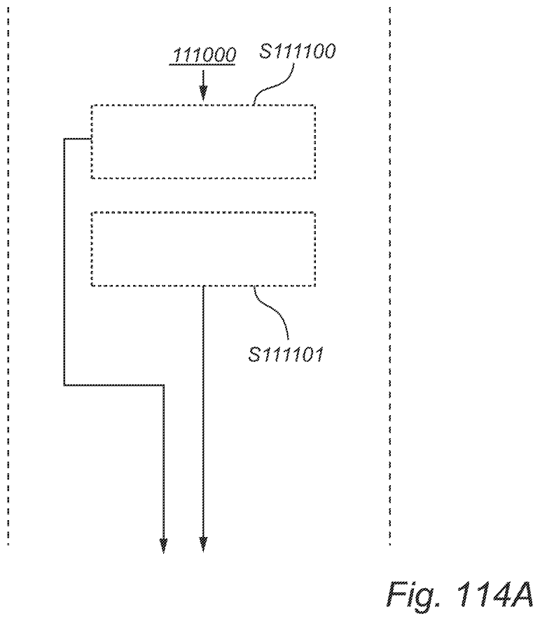

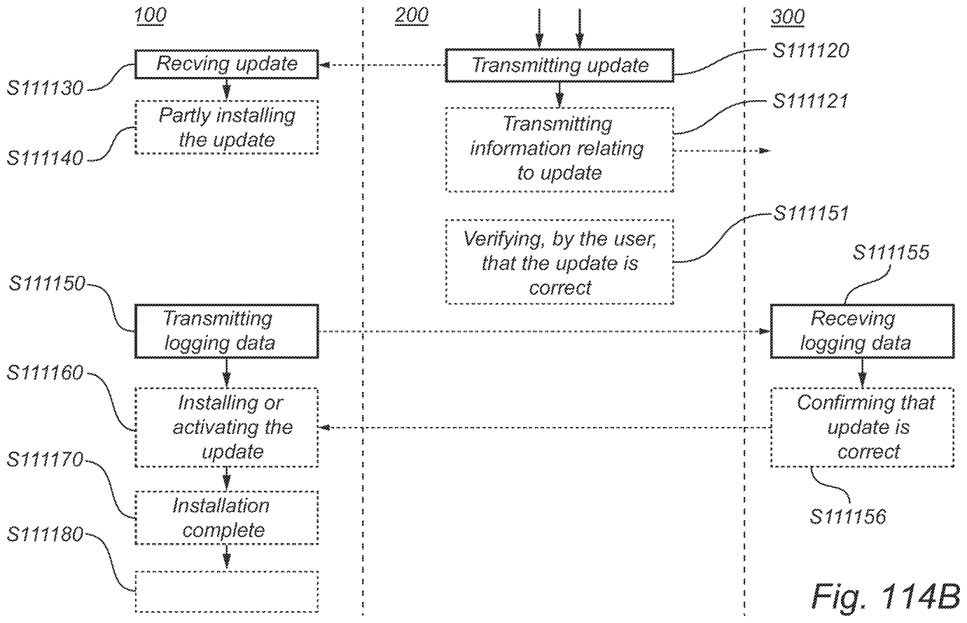

In aspect 314SE, a method updating a control program of an internal computing unit comprised in an implant is provided. FIGS. 114A-B and 113A-C shows embodiments of this aspect.

Generally, aspect 314SE defines a method, as shown in FIG. 114A-B, and a system, as shown in FIGS. 113A-C. The system generally comprises an implant 100, an external device 200 and a second external device 300, which may be any of the implants 100 or external devices 200, 300, 400 described with reference to aspects one through twenty-eight, as shown in FIGS. 110A-C.

When updating a control program of the internal computing unit, it may be beneficial to transmit a confirmation to a user or to an external device or system. Such a method is now described with reference to FIGS. 114A-B, with system reference to FIG. 113A-C. In FIGS. 114A-114B, optional method steps have been indicated with dashed lines.

The method 111000 for updating a control program 110/112/114 for an implant 100 according to any of the embodiments described with reference to aspects one to twenty-eight, wherein the implant is adapted for communication with a first external device and a second external device, which may comprise receiving S111130, by an internal computing unit or internal communication unit comprised in the implant 100, an update or configuration to the control program 110/112/114 from the first external device, wherein the update is received using a first communication channel W1/C1; installing S111140/S11160/S111170, by the internal computing unit, the update; and transmitting S111150, by the internal computing unit, logging data relating to the receipt of the update or configuration and/or logging data relating to an installation of the update to the second external device using the second communication channel W4; wherein the first and the second communication channels are different communication channels. By using a first and a second communication channels, in comparison to only using one, the security of the updating may be improved as any attempts to update the control program 110/112/114 will be logged via the second communication channel W4, and thus, increasing the chances of finding incorrect or malicious update attempts.

The update or configuration comprises a set of instructions for the control program, and may, for examples comprise a set of predefined program steps as described above with reference to aspect 312SE. The configuration or update may comprise a value for a predetermined parameter.

In some examples, the method further comprises confirming S111151, S1111156, by a user or by an external control unit, that the update or configuration is correct based on the received logging data.

The logging data may be related to the receipt of the update or configuration, and the internal computing unit is configured to install S111160 the update or configuration in response to receipt of a confirmation that the logging data relates to a correct set of instructions. In this way, the internal computing unit may receive S111130 data, transmit S11150 a logging entry relating to the receipt, and then install S111160 the data in response to a positive verification that the data should be installed.

In another example, or in combination with the one described above, the logging data is related to the installation or the update or configuration. In this example the logging data may be for information purposes only and not affect the installation, or the method may further comprise activating S111180 the installation in response to the confirmation that the update or configuration is correct.

If the update or configuration is transmitted to the internal computing unit in one or more steps, the verification as described above may be performed for each of the steps.

The method may further comprise, after transmitting the logging data to the second external device, verifying S111156 the update via a confirmation from the second external device via the second communication channel.

### Aspect 315SE eHealth Sleeping Internal Control Unit—Sleep Mode for Internal Controller—Embodiments of Aspect 315SE of the Disclosure

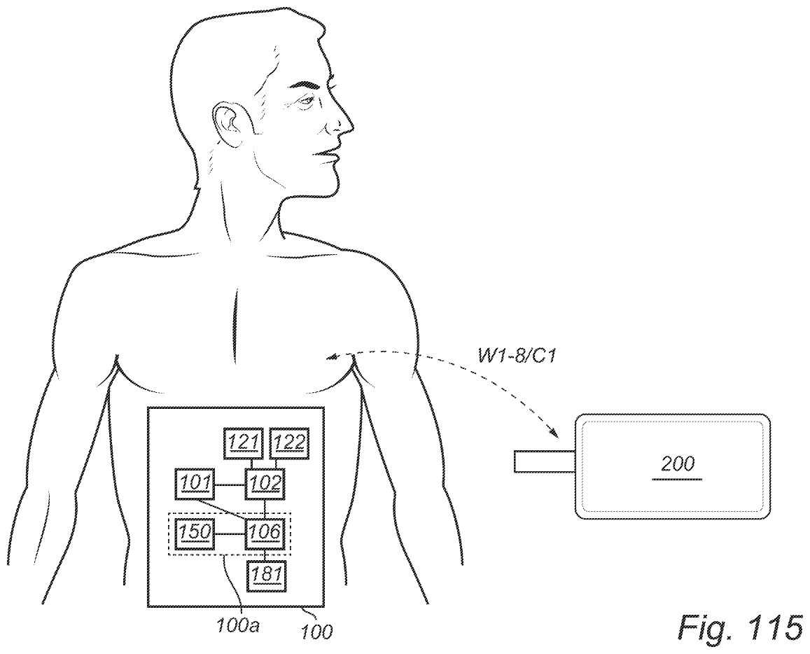

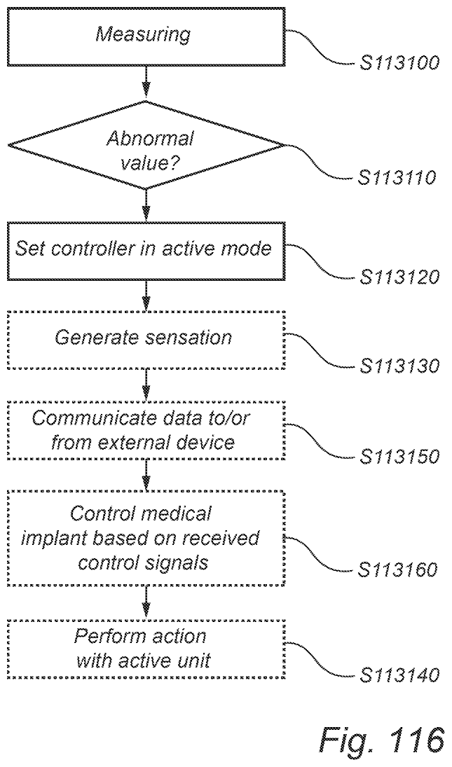

In aspect 315SE, a method and a system for controlling an implant is provided. FIGS. 115 and 116 show embodiments of this aspect.

Generally, aspect 315SE defines a method, as shown in FIG. 116, and a system, as shown in FIG. 115. The system generally comprises an implant 100 and an external device 200, which may be any of the implants 100 or external devices 200, 300, 400 described with reference to aspects one through twenty-nine. The internal control unit 100a is in this example shown as separate from the communication unit 102 but may also beneficially be comprised in the communication unit 102, as for example shown in some aspects described herein.

As shown in FIG. 115, the internal control unit 100a comprises a processor 106, the processor having a sleep mode and an active mode, and a sensor 150, wherein the sensor 150 is configured to periodically measure a physical parameter of the patient, and wherein the internal control unit 100a is further configured to, in response to a sensor measurement preceding a predetermined value, setting the processing unit 106 in an active mode. That is, the internal control unit 100a may “wake up” or be set in an active mode in response to a measurement from, for example, the body. A physical parameter of the patient could for example be a local or systemic temperature, saturation/oxygenation, blood pressure or a parameter related to an ischemia marker such as lactate.

By sleeping mode, it is meant a mode with less battery consumption and/or processing power used in the processing unit 106, and by “active mode” it may be meant that the processing unit 106 is not restricted in its processing.

The sensor 150 may, for example, be a pressure sensor. The pressure sensor may be adapted to measure a pressure in an organ of a patient, a reservoir of the implant or a restriction device of the active unit 101. The sensor 150 may be an analog sensor or a digital sensor, i.e. a sensor 150 implemented in part in software. In some examples, the sensor is adapted to measure one or more of a battery or energy storage status of the implant and a temperature of the implant. In this way, the sensor 150 may periodically sense a pressure of the implant or of the patient and set the processing unit 106 in an active mode if the measured pressure is above a predetermined value, less than a predetermined value or outside of a defined range. Thus, less power, i.e. less of for example a battery or energy storage comprised in the implant, may be used, thereby prolonging the lifetime of the implant 100 or increasing the time between charging occasions of the implant 100.

In some examples, the processor 106, when in set in the active mode, may cause a sensation generator 181 connected to the implant, comprised in the implant or comprised in an external device 200, 300, to generate a sensation detectable by a sense of the patient. For example, the processor may cause the sensation generator to generate a sensation in response to a measure battery status, for example that the battery is above or below a predetermined level, that a measured pressure is above or below a predetermined level, or that another measured parameter has an abnormal value, i.e. less than or exceeding a predetermined threshold or outside of a predetermined interval. The sensation generator 181 has been described in further detail earlier in this description. In this way, the patient in which the implant is implanted may be informed of changes or other information regarding the implant.

The processing unit 106 may be configured to perform a corrective action for the active unit 101 in response to a measurement being below or above a predetermined level. Such a corrective action may, for example, be increasing or decreasing a pressure, increasing or decreasing electrical stimulation, increasing or decreasing power, or another action.

The internal control unit 100a may comprise or be connected to an internal communication unit 102 or a signal transmitter 102a, and wherein the processing unit 106 is configured to transmit data relating to the measurement via the internal communication unit 102 or the internal signal transmitter 102a. The transmitted data may be received by an external device 200.

The external device 200 may have an external communication unit 290. The external device may comprise a signal provider 280 for providing a wake signal to the internal control unit. In some examples, the signal provider comprises a coil or magnet 281 for providing a magnetic wake signal.

The implant 100 may implement a corresponding method for controlling a medical implant when implanted in a patient, which will now be described with reference to FIG. 116. The method 113000 comprises measuring S113100, with a sensor of a controller connected to or comprised in the medical implant, a physiological parameter of the patient or a parameter of the medical implant, and, in response to S113110 a sensor measurement having a value outside of a predetermined interval, setting S113130, by the controller, a processor of the controller from a sleep mode to an active mode. The measuring S113100 may be carried out periodically. By “a value outside of a predetermined interval” it may be meant a measured value exceeding or being less than a predetermined value, or a measured value being outside a defined range or an interval determined by a control unit. The method may further comprise generating S113130, with a sensation generator as described above, a sensation detectable by the patient.

In some examples, the generating comprises requesting, by the processor, the sensation generator to generate the sensation.

The method may further comprise to perform a S113150 medical intervention or an action with the active unit in response to a sensor measurement having a value outside of a predetermined interval, preferably after the processing unit has been set in the active mode.

The method 113000 may further comprise a step of communicating S113150 data to the external device. The data may, for example, be related to the measured value. In some examples, the external device may respond S113150 to the communicated data with a control signal. The method 113000 may further comprise the step of controlling S113160 the implant based on the received control signal.

### Aspect 316SE Relaying of Instructions—Relaying of Instructions—Embodiments of Aspect 316SE

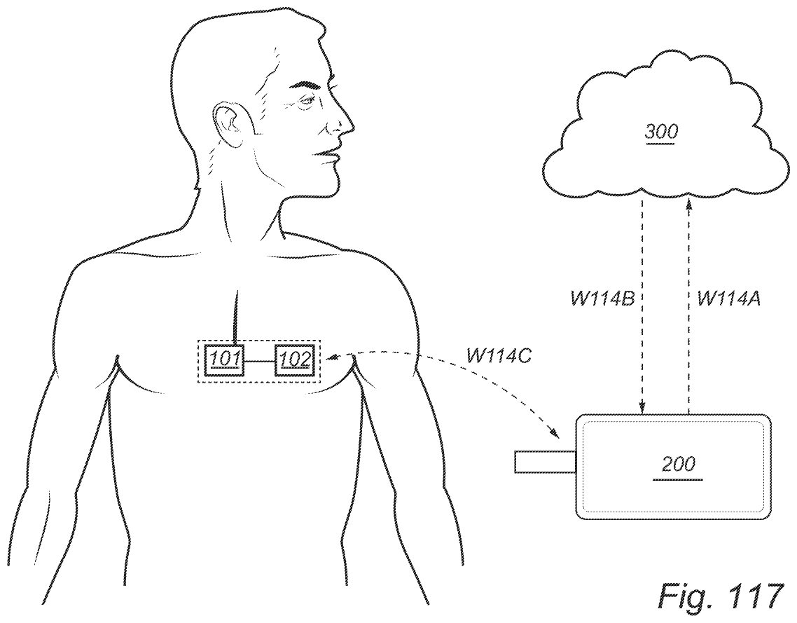

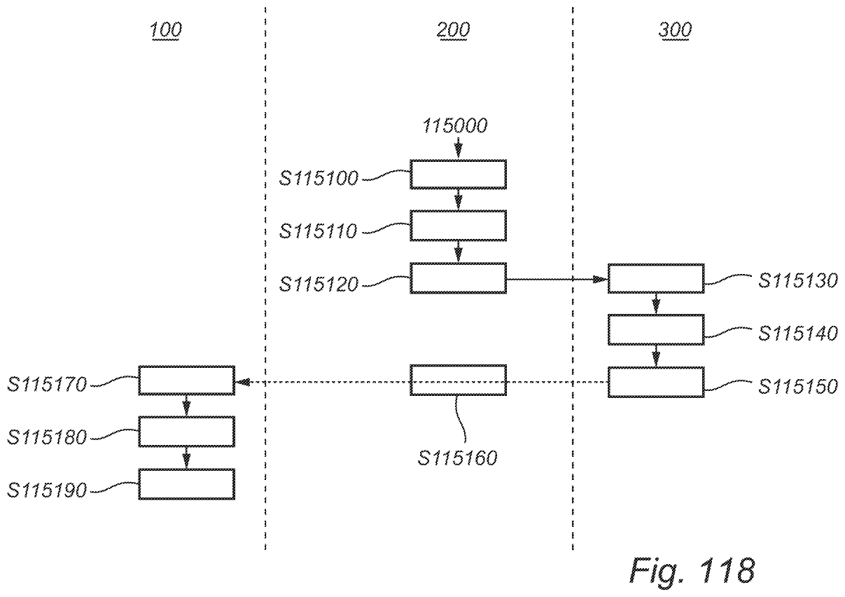

In aspect 316SE, a system, and a method for communication between an external device 200 and an implant 100 is provided. FIGS. 117 and 118 shows embodiments of this aspect.

Generally, aspect 316SE defines a system, as shown in FIG. 117 and a method shown in FIG. 118. The system shown in FIG. 117 is generally adapted to perform the method described with reference to FIG. 118. The implant 100 and the external devices 200, 300 may be any of the implants 100 or external devices 200, 300, 400 described with reference to aspects one through thirty, further comprising the features described below.

The system shown in FIG. 117 comprises an implant 100, a first external device 200, and a second external device 300. The implant comprises a communication unit 102 (which could also be called an internal control unit, or the communication unit 102 may be comprised in an internal control unit, in some examples the communication unit may comprise a processor) and an active unit 101. The communication unit 102 is adapted to receive an instruction from an external device 200 over the communication channel W11114C and run the instruction to control a function of the implant, such as a function of the active unit 101. The communication channel may be any type of communication channel, such as any of the wireless connection W1-W8 or the conductive connection C1-C3 described with reference to aspects 1 through thirty. For example, the wireless connection may comprise at least one of the following protocols:
Radio Frequency type protocol
RFID type protocol
WLAN type protocol
Bluetooth type protocol
BLE type protocol
NFC type protocol
3G/4G/5G type protocol
GSM type protocol
Bluetooth 5.

The first external device 200 is adapted to receive, such as through a user interface, or determine an instruction to be transmitted to the implant 100. The determination of the instruction may, for example, be based on received data from the implant 100, such as measurement data or data relating to a state of the implant, such as a battery status or a free memory status. The first external device 200 may be any type of device capable of transmitting information to the implant 100 and capable of determining or receiving an instruction to be transmitted to the implant 100. In a preferred embodiment, the first external device 200 is a hand-held device, such as a smartphone, smartwatch, tablet etc. handled by the patient, having a user interface for receiving an instruction from a user, such as the patient or a caregiver.

The first external device 200 is further adapted to transmit the instruction to a second external device 300 via communication channel W114B. The second external device 300 is adapted to receive the instruction, encrypt the instruction using an encryption key, and then transmit the encrypted instruction to the implant 100. The implant 100 is configured to decrypt the received instruction. The decryption may be performed using a decryption key corresponding to the encryption key. The encryption key, the decryption key and methods for encryption/decryption and exchange of keys may be performed as described in the “general definition of features” or as described with reference to aspects two to nine or thirteen to fifteen. Further, there are many known methods for encrypting data which the skilled person would understand to be usable in this example.

The second external device may be any computing device capable of receiving, encrypting, and transmitting data as described above. For example, the second external device may be a network device, such as a network server, or it may be an encryption device communicatively coupled to the first external device.

The instruction may be a single instruction for running a specific function or method in the implant, a value for a parameter of the implant, or a set of sub-steps to be performed by a processor or computing unit comprised in the implant.

In one embodiment the instruction for controlling a function of the implant 100 is received at the first external device but transmitted to the implant via the second external device 300. By having a second external device encrypting the instruction before transmitting it to the implant, the instruction may be verified by the external device and the first external device may function so as to relay the instruction. In some alternatives, the second external device 300 may transmit the instruction directly to the implant 100. This may provide an increased security as the instruction sent to the implant may be verified by the second external device, which, for example, may be a proprietary device managed by the medical professional responsible for the implant. Further, by having the second medical device verifying and encrypting the instruction, the responsibility authenticity and/or correctness of the instruction lies with the second external device, which may be beneficial for regulatory purposes, as the first external device may not be considered as the instructor of the implant.

Further, the second external device 300 may verify that the instruction is correct before encrypting or signing and transmitting it to the implant 100. The second external device may, for example, verify that the instruction is correct by comparing the instruction with a predetermined set of instructions, and if the instruction is comprised in the predetermined set of instructions determine that the instruction is correct. If the instruction comprises a plurality of sub-steps, the second external device may determine that the instruction is correct if all the sub-steps are comprised in the predetermined set of instructions. If the instruction comprises a value for a parameter of the implant, the second external device may verify that the value is within a predetermined range for the parameter.

The second external device may be configured to reject the instruction, i.e. to not encrypt and transmit the instruction to the implant, if the verification of the instruction would fail. For example, the second external device determines that the instruction or any sub step of the instruction is not comprised in the predetermined set of instructions, or if a value for a parameter is not within a predetermined interval, the second external device may determine that the verification has failed.

In some embodiments, the implant may be configured to verify the instruction. The verification of the instruction may be performed in the same way as described with reference to the second external device above. If the verification is performed by comparing the instruction or any sub steps of the instruction with a predetermined set of instructions, the implant may comprise a predetermined set of instructions. The predetermined set of instructions may, for example, be stored in an internal memory of the implant. Similarly, the implant may store predetermined reference intervals for any parameter that can be set, and the implant may be configured to compare a received value for a parameter to such a predetermined reference interval. If the verification of the instruction would fail, the implant may be configured to reject the instruction, i.e. not run the instruction at the implant.

In an alternative to encrypting and decrypting the instruction, the instruction may be signed by the second external device using a cryptographic hash, and the implant may be configured to verify that the signature is correct before running the instruction.

A corresponding method will now be described with reference to FIG. 118. FIG. 118 shows a flowchart for a method for transmitting instructions from a first external device to an implant. The instruction may relate to a function of the implant, such as an instruction to run a function or method of the implant, or to set a value of a parameter of the implant.

The method comprises: transmitting S115120 an instruction for the implant from the first external device 200 to a second external device 300, the instruction relating to a function of the implant 100, encrypting S115140, at the second external device 300 and using a first encryption key, the instruction into an encrypted instruction, and transmitting S115150 the encrypted instruction from the second external device 300 to the implant 100, decrypting S115170, at the implant, the instructions using a second encryption key corresponding to the first encryption key.

The instruction may be any type of instruction for controlling a function of the implant. For example, the instruction may be an instruction to run a function or method of the implant, an instruction comprising a plurality of sub steps to be run at the implant, or a value for a parameter at the implant. The first external device may, for example, receive the instruction from a user via a user interface displayed at or connected to the first external device. In another example, the first external device may determine the instruction in response to data received from the implant, such as measurement data, or from another external device. Thus, in some examples, the method may further comprise receiving S115100, at the first external device, an instruction to be transmitted to the implant. The method may further comprise displaying a user interface for receiving the instruction. In another example, the method comprises determining S115110, at the first external device, an instruction to be transmitted to the implant.

In some embodiments, the transmitting of the encrypted instruction from the second external device to the implant comprises transmitting S115150 the encrypted instruction from the second external device to the first external device, and transmitting S115160 the encrypted instruction from the first external device to the implant. In other words, the first external device may relay the encrypted instruction from the second external device to the implant, preferably without decrypting the instruction before transmitting it.

Additionally or alternatively, the transmitting of the encrypted instruction from the second external device to the implant comprises transmitting the encrypted instruction from the second external device to a third external device, and transmitting the encrypted instruction from the third external device to the implant. Accordingly, the third external device act as relay between the implant and the second external device wherein the third external device does not decrypt the instruction before transmitting it.

Accordingly, in some embodiments the first external device is used for transmitting instructions to the second external device whereas the third external device is used for relaying the encrypted instruction from the second external device to the implant. For example, the first external device is an advanced user device such as a smartphone whereas the third external device is a simpler device or a proprietary relaying device which may be configured to essentially only act as a relaying device from the second external device to the implant. The third external device comprises a transmitter for wirelessly transmitting the encrypted instructions to the implant. Alternatively or additionally, the third external device is configured to be in conductive or capacitive connection with the implant and transmit the encrypted instruction to the implant. The third external device further comprises a receiver configured to receive the encrypted instructions from the second external device. The third external device communicates with the second external device using a wireless or wired communication channel.

In some embodiments the implant is configured to directly receive the encrypted instructions from the second external device, e.g. over a wireless communication channel such as a mobile network communication channel. To this end the implant comprise a receiver configured to receive the encrypted instructions transmitted from the second external device. The second external device may be a remote server or a cloud server.

The method may further comprise to, at the implant, running S115190 the instruction or performing the instruction. The running of the instruction may be performed by an internal computing unit or a processor comprised in the implant, and may, for example, cause the internal computing unit or processor to instruct an active unit of the implant to perform an action.

The method may further comprise verifying S115130, at the second external device, that the instructions are correct. The verifying may be performed as described above with reference to the system and FIG. 117.

The method may further comprise verifying S115180, at the implant, that the instructions are correct. The verifying may be performed as described above with reference to the system and FIG. 117.

The method may further comprise authenticating the connection between the first external device and the implant over which the encrypted instruction is to be transmitted. The authentication may be performed as described in any of aspects one through thirty.

### Aspect 317SE Energy General Microphone—Microphone Sensor—Embodiments of Aspect 317SE of the Disclosure

In aspect 317SE there is provided an implantable controller for controlling an implant based on registered microphone signals. Generally, aspect 316SE defines an implantable controller for controlling an energized implant, and a method for authenticating a method of authenticating an energized implant implanted in a patent.

With reference to FIG. 18A, FIG. 18B and FIG. 19 there is provided a medical implant 100. The implantable controller may be connected to or comprised in the medical implant 100. The controller may comprise or be connected to a sensor 150 wherein the sensor 150 is at least one microphone sensor 150 configured to record acoustic signals. For instance, the controller may be configured to register a sound related to at least one of a bodily function of the patient and a function of the implant 100. The controller may further comprise or be in communication with a computing unit 106 wherein the computing unit 106 is configured to derive at least one of a pulse of the patient from the registered sound related to a bodily function, information related to the patient urinating from the registered sound related to a bodily function, information related to a bowel activity of the patient from the registered sound related to a bodily function, and information related to a functional status of the implant from the registered sound related to a function of the implant. To this end the computing unit 106 may be configured to perform signal processing on the registered sound (e.g. on a digital or analog signal representing the registered sound) so as to derive any of the above mentioned information related to a bodily function of the patient or a function of the implant 100. The signal processing may comprise filtering the registered sound signals of the microphone sensor 150.

The implantable controller may further comprise at least one implantable housing for sealing against fluid wherein the computing unit 106 and the microphone sensor 150 are placed inside of the housing. For instance, the implant 100 may comprise the controller and the microphone sensor 150 inside an implantable housing for sealing against fluid. Accordingly, at least the controller and the microphone sensor 150 does not come into contact with bodily fluids when implanted which ensures proper operation of the controller and the microphone sensor 150.

In some implementations, the computing unit 106 is configured to derive information related to the functional status of an operation device of the implant, from the registered sound related to a function of the implant. Accordingly, the computing unit 106 may be configured to derive information related to the functional status of at least one of: a motor, a pump and a transmission of the operation device of the implant from, the registered sound related to a function of the implant.

The controller may comprise a transceiver wherein the controller is configured to transmit a parameter derived from the sound registered by the at least one microphone sensor 150 using the transceiver. For example, the controller is provided externally of the implant 100 and transmits the derived parameter to the implant 100 or the controller is comprised in the implant 100 and the transceiver is a transceiver of a communication unit 102 of the implant 100 wherein communication unit 102 of the implant 100 is configured to transmit the parameter wirelessly or conductively to an external device 200 or wirelessly to a second external device 300.

Aspect 316SE further relates to a method of authenticating at least one of an energized implant implanted 100 in a patent, an external device 200 and a connection between the implant 100 and the external device. The method is performed in a system comprising the energized implant 100 and an external device 200, the energized implant 100 comprising at least one microphone sensor 150, and a transmitter, and the external device 200 comprising a receiver and a computing unit. The method comprising the steps of registering a sound related to at least one of a bodily function and a function of the implant 100, using the at least one microphone sensor 150, transmitting a signal derived from the registered sound, using the transmitter, receiving, in the external device, the signal derived from the registered sound, using the receiver, and comparing, in the external device 200, a parameter derived from the received signal with a reference parameter, using the computing unit. The registered sound may be related to the pulse of the patient. Based on the comparison at least one of an energized implant implanted 100 in a patent, an external device 200 and a connection between the implant 100 and the external device 200.

The aforementioned method is exemplified with further reference to FIG. 88 and FIG. 89. At S5602 a sound related to at least one of a bodily function and a function of the implant 100, using the at least one microphone sensor 150 is registered as a parameter. The method then goes to S5604b wherein a signal derived from the registered sound is transmitted to the external device 200 and compared to a reference parameter. Optionally, upon comparing the parameter derived from the received signal with a reference parameter at S5604b the method may go to S5605 involving authenticating at least one of an energized implant implanted 100 in a patent, an external device 200 and a connection between the implant 100 and the external device 200 on the basis of the comparison. For example, if the difference between the derived parameter and reference parameters is below a predetermined threshold or within an expected range of differences the energized implant 100 is authenticated. The method may further comprise the step of receiving at the receiver of the external device a parameter to be used as a reference parameter. For instance, the parameter may be received from a sensor external to the patient. Such as a pulse sensor, microphone or temperature sensor. In other words, aspect 317SE is similar to aspect 256SE wherein the sensed parameter is a registered sound.

Alternatively, the comparison of the parameter derived from the sound with the reference parameter may be performed by the computing unit 106 in the in the energized implant 100 as opposed to the in the computing unit of the external device 200. Accordingly, another method is provided wherein the method is a method of authenticating at least one of energized implant 100 implanted in a patent, an external device 200 and a connection between the implant 100 and the external device 200, performed in a system comprising the energized implant 100 and an external device 200, the energized implant 100 comprising at least one microphone, a receiver, and a computing unit 106, and the external device 200 comprising a transmitter. The method comprising the steps of registering a sound related to at least one of: a bodily function and a function of the implant 100, using the at least one microphone, deriving a parameter from the sound using the computing unit 106, receiving, in the energized implant 100, a reference parameter, from the external device 200, using the receiver, and comparing, in the energized implant 100, the parameter derived from the sound with the received reference parameter, using the computing unit 106. The registered sound may be related to a pulse of the patient wherein the reference parameter is related to the pulse of the patient and/or another bodily function of the patient. A sound related to at least one of a bodily function and a function of the implant 100, using the at least one microphone of the implant 100 is registered as a parameter at S5602. The method may then go to S5604 comprising receiving a reference parameter from the external device 200 at the implant 100 using the receiver of the implant 100 and comparing the parameter derived from the sound with the received reference parameter, using the computing unit 106 of the implant 100. The method may further comprise the step S5605 of authenticating the energized implant 100 and/or external device 200 and/or connection therebetween on the basis of the comparison performed in the computing unit 106 of the implant 100. For instance, if the comparison yields that the derived parameter and the reference parameter are similar the implant 100 may be authenticated. Additionally or alternatively, the method may further comprise receiving, at a receiver of the external device 200, a parameter to be used as reference parameter wherein the parameter is received form a sensor external to the patient. The sensor may be integrated with the external device 200 or provided separately from the external device.

The authentication of the external device is performed by the energized implant, i.e. the step of comparing two parameters for the purpose of authentication is performed by the computing unit of the energized implant.

The authentication of the communication may be performed by the energized implant or by the external device, i.e. the step of comparing two parameters for the purpose of authenticating the communication session may be performed by the computing unit of the energized implant or by the computing unit of the of the external device.

### Aspect 318SE Energy Appetite Control Microphone—Microphone Sensor for Appetite Control—Embodiments of Aspect 318SE of the Disclosure

In aspect 318SE there is provided an implantable controller for controlling an energized implant for stretching the stomach wall of a patient to create satiety based on registered sound sensor signals. Generally, aspect 318SE defines an implantable controller for controlling an energized implant to create satiety, a system for controlling an energized implant for stretching the stomach wall of a patient to create satiety and a method for controlling an energized implant for stretching the stomach wall of a patient to create satiety. With reference to FIG. 18A there is illustrated an energized implant 100. The aforementioned implantable controller may be comprised in the energized implant 100 or provided externally and being configured to communicate with energized implant 100. With further reference to FIG. 18B it is illustrated that the energized implant 100 may further computing unit 106. The computing unit 106 may be comprised in the controller.

With further reference to FIG. 19 it is illustrated that the controller and/or implant 100 may comprise a sensor 150 wherein the sensor 150 in aspect 318SE is at least one microphone sensor 150 configured to register a sound related to the patient swallowing. The computing unit 106 is configured to derive a parameter related to the patient swallowing from the sound registered by the microphone sensor 150. The computing unit 106 may be configured to derive a parameter related to one or more of the size and/or shape and/or viscosity of a swallowed contents. Additionally or alternatively, the computing unit 106 is configured to determine if a swallowed content is a liquid or a solid and/or to determine an accumulated amount of swallowed content over a time period. Accordingly, the stretching of the stomach wall may be controlled depending on when and/or what the patient is swallowing so as to e.g. create satiety when the patient is eating or has eaten a predetermined threshold amount of food.

For example, the computing unit 106 may be configured to analyze acoustic properties of the registered sound so as to derive a parameter according to the above. Wherein the acoustic properties may comprise at least one of the frequency content of the registered sound, the magnitude or amplitude of the registered sound and the duration of the registered sound.

In some implementations, the controller further comprises and/or is in communication with a transmitter wherein the controller is configured to transmit the parameter derived from the sound registered by the at least one microphone sensor 150 using the transmitter. The transmitter may be a part of the communication unit 102 of an implant 100 which comprises or is in communication with the controller. For instance, the computing unit may be configured to transmit the parameter derived from the registered sound to an external device 200 using the transmitter. Additionally or alternatively, the controller comprises and/or is on communication with a receiver wherein the controller is configured to receive a signal from an external device. The receiver may be a part of the communication unit 102 of an implant 100 which comprises or is in communication with the controller. For instance, the computing unit 106 may be configured to receive a control signal from an external device.

In some implementations the computing unit 106 is further configured to generate a control signal for controlling the energized implant 100 for stretching the stomach wall of a patient on the basis of at least one of the derived parameter related to the patient swallowing, the signal received from the external device 200, and a combination of the derived parameter related to the patient swallowing and the signal received from the external device 200. Accordingly, the implant 100 may be controlled to stretch the stomach wall of a patient so as to create satiety based on the derived parameter related to the patient swallowing or the signal received from the external device 200. That is, satiety may be created based on the patient swallowing or the contents which the patient swallows. Additionally or alternatively, satiety is created based on a signal received from the external device 200. For instance, the patient may input to the external device 200 information associated with the content which the patient is eating or the external device may detect that the patient is eating wherein associated information may be conveyed as a signal to the implantable controller for controlling the stretching of the stomach wall.

Aspect 318SE further relates to a system for controlling an energized implant 100 for stretching the stomach wall of a patient to thereby create satiety, the system comprising an implantable controller for controlling the energized implant and an external device 200. The system further comprises at least one microphone sensor 150 configured to register a sound related to the patient swallowing, a computing unit 106 configured to derive a parameter related to the patient swallowing from the registered sound, a transmitter configured to transmit the derived parameter, a receiver configured to receive control signals from the external device. Additionally, the system comprises the external device 200 which comprises a receiver configured to receive a parameter derived from a sound related to the patient swallowing, a computing unit 106 configured to generate a control signal on the basis of the received parameter, and a transmitter configured to transmit the control signal to the implantable controller for controlling the energized implant for stretching the stomach wall of a patient to thereby create satiety.

Accordingly, the external device 200 receives a derived parameter and generates a control signal based on the derived parameter, wherein the external device 200 transmits control signal back to the implantable controller for controlling the energized implant 100 according to generated control signals.

The computing unit 106 in the external device 200 may be configured to derive a parameter related to the size and/or shape and/or viscosity of swallowed contents and/or to determine if a swallowed content is a liquid or a solid on the basis of the received parameter.

The computing unit 106 of the external device 200 may be configured to determine an accumulated amount of swallowed content over a time period and the computing unit may further be configured to generate the control signal on the basis of the accumulated amount of swallowed content over a time period. For instance, for larger accumulated amounts of swallowed contents the stretching of the stomach wall is increased so as to increase the level of satiety.

Aspect 318SE further relates to a method in an implantable controller for controlling an energized implant 100 for stretching the stomach wall of a patient to thereby create satiety, when implanted in a patient, the implantable controller comprises at least one microphone sensor 150 and a computing unit 106. The method comprises the steps of registering a sound related to the patient swallowing, using the at least one microphone and deriving a parameter related to the patient swallowing from the sound, using the computing unit. Any structural features described in relation to the at least one microphone, controller or computing unit, may have the corresponding steps in this method. It is noted that aspect 318SE relates to all possible combinations of features recited in the embodiments, e.g. the method may comprise the step of controlling the energized implant 100 for stretching the stomach wall of a patient, using the computing unit, on the basis of at least one of the derived parameter related to the patient swallowing, the signal received from the external device, and a combination of the derived parameter related to the patient swallowing and the signal received from the external device 200.

Aspect 318SE further relates to a method of authenticating at least one of an implantable controller for controlling an energized implant 100 for stretching the stomach wall of a patient to create satiety, an external device 200, and a connection between the implant 100 and the externa device 200. The method is performed in a system comprising the energized implant 100 and an external device 200, the energized implant 100 comprising at least one microphone, and a transmitter, and the external device 200 comprising a receiver and a computing unit. The method comprises the steps of registering a sound related to the patient swallowing, using the at least one microphone 150, and transmitting a signal derived from the registered sound, using the transmitter, receiving, in the external device 200, the signal derived from the registered sound, using the receiver, and comparing, in the external device 200, a parameter derived from the received signal with a reference parameter, using the computing unit. With further reference to FIG. 88 and FIG. 89 the at least one microphone is used to register a sound related to the patient swallowing at S5602 and at S5604b the signal is transmitted to the external device 200 wherein the external device 200 compares a parameter derived from the received signal with a reference parameter at S5604. The method may optionally comprise the further step S5605 of authenticating the energized implant 100 on the basis of the comparison. For instance, if the difference between the parameter derived from the received signal and the reference parameter is below a predetermined threshold the at least one of the energized implant 100, external device 200 and connection between the energized implant 100 and external device 200 is authenticated.

Additionally or alternatively the method may further comprise receiving, at a receiver of the external device 200, a parameter to be used as reference parameter wherein the parameter may be received form a sensor external to the patient. The sensor may be integrated with the external device 200 or provided separately from the external device. The step of receiving the parameter from a sensor external to the patient may comprise receiving the parameter from a sensor configured to sense the patient swallowing. Wherein this external sensor is separate from the at least one microphone of the controller. The step of receiving a parameter to be used as reference parameter at the external device 200 may comprise receiving input from the patient. For instance, the patient may input to the external device 200 information indicating whether or not the patient is eating and optionally whether the food is fluid or solid. Accordingly, the implant 100, external device 200 or connection therebetween may be authenticated based on the at least one microphone of the controller determining that the patient is swallowing content corresponding to the reference parameter received at the external device 200.

Aspect 318SE relates at least partially to sensing that the patient is swallowing (e.g. the amount of the matter the patient is swallowing and whether the swallowed matter is fluid or solid) using at least one microphone sensor 150 implanted in the patient. It is understood that the registered sound which is associated with the patient swallowing may be used for device synchronization and/or authentication analogously to the parameters of aspect 248SE and 256SE. The patient swallowing may also be used as sensation in aspect 258SE.

The authentication of the external device is performed by the energized implant, i.e. the step of comparing two parameters for the purpose of authentication is performed by the computing unit of the energized implant.

The authentication of the communication may be performed by the energized implant or by the external device, i.e. the step of comparing two parameters for the purpose of authenticating the communication session may be performed by the computing unit of the energized implant or by the computing unit of the of the external device.

### Aspects 396-402

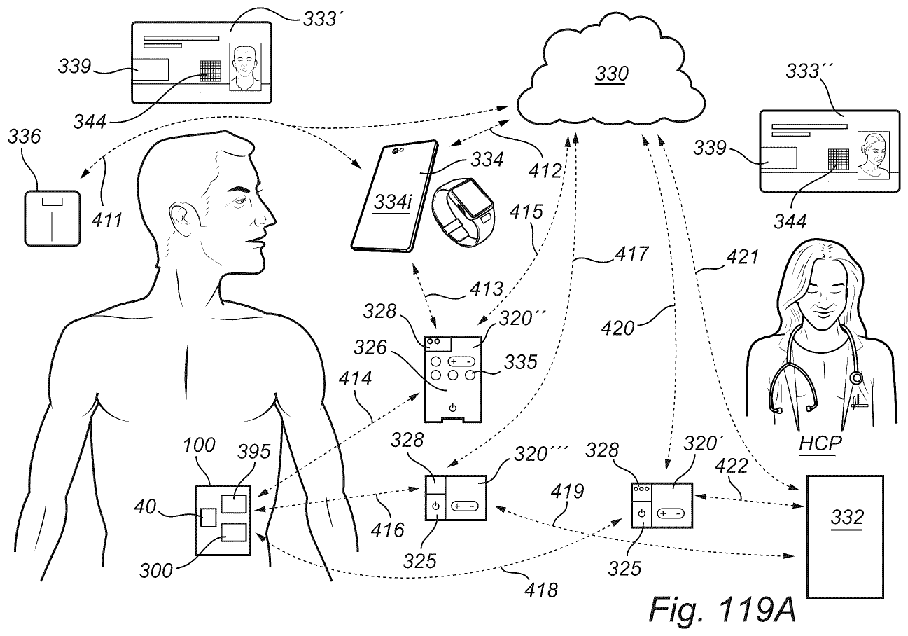

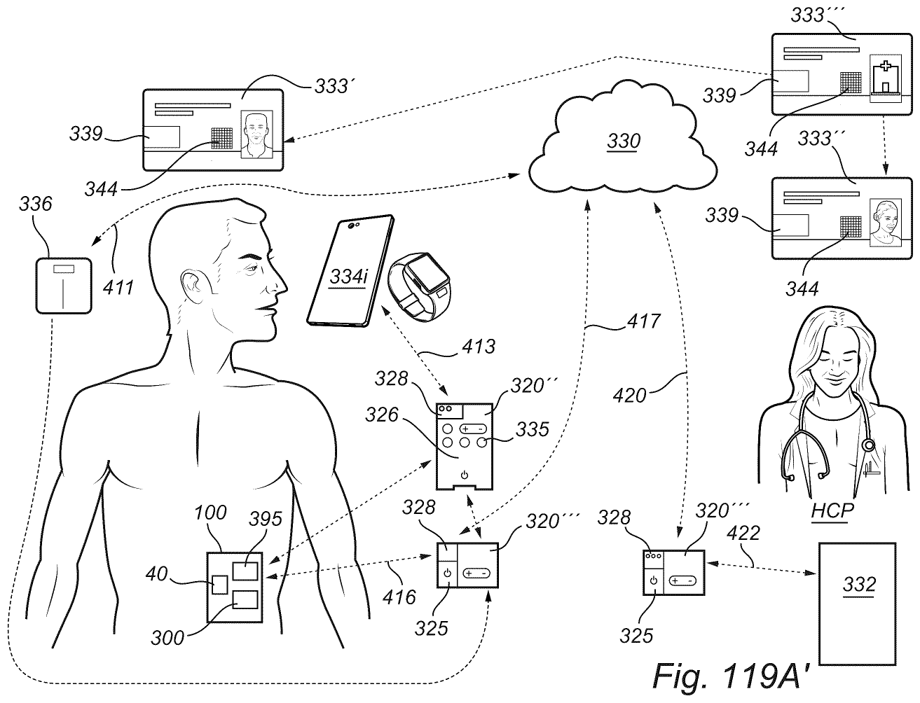

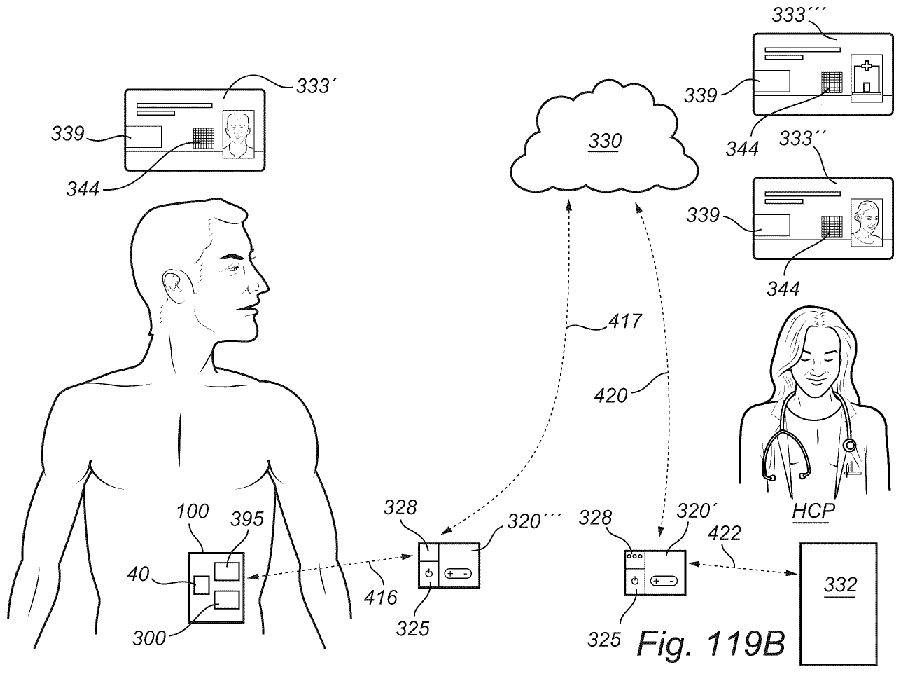

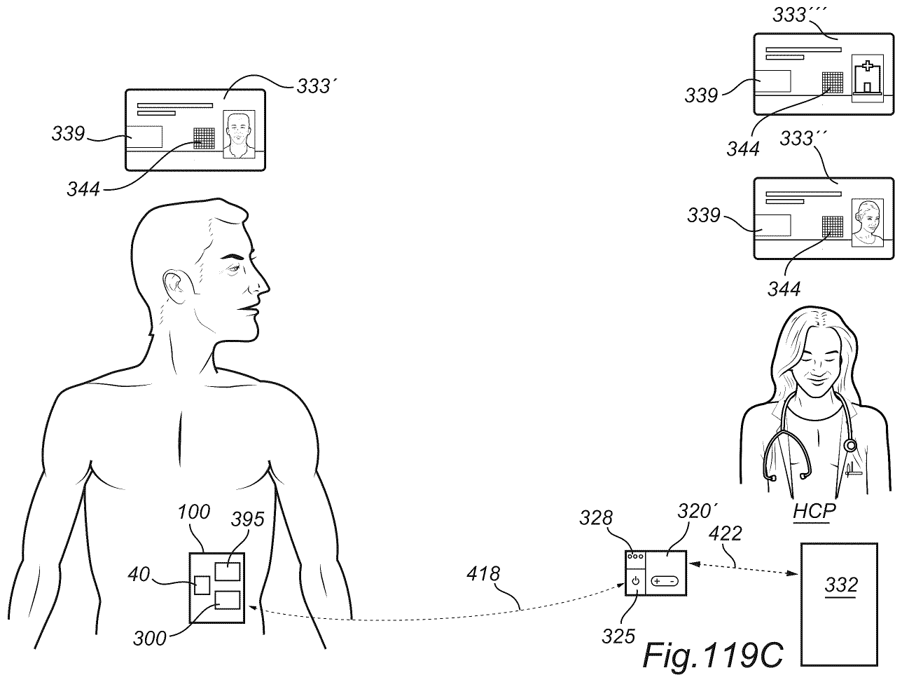

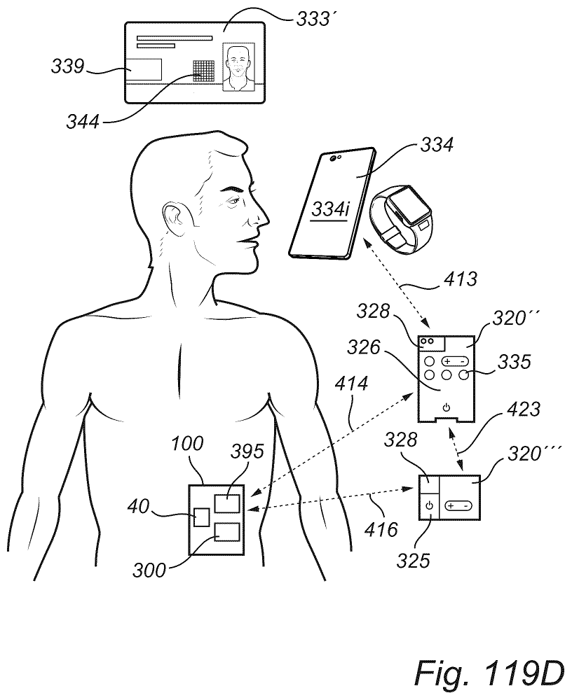

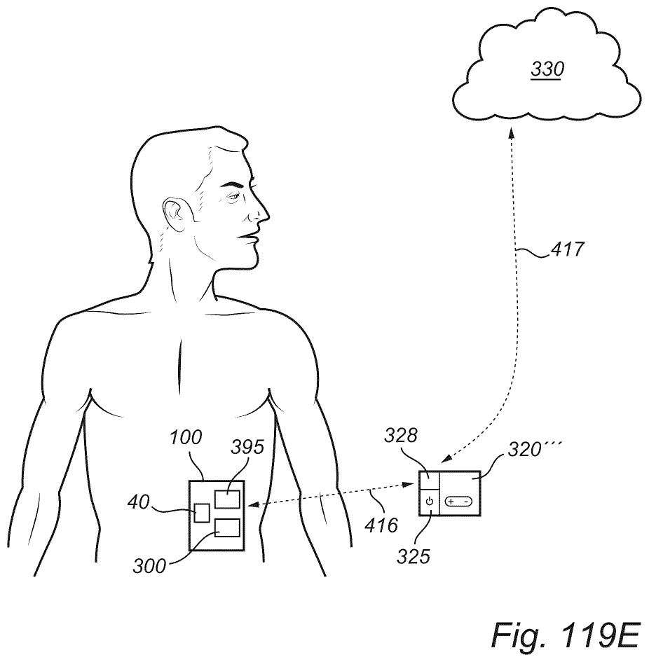

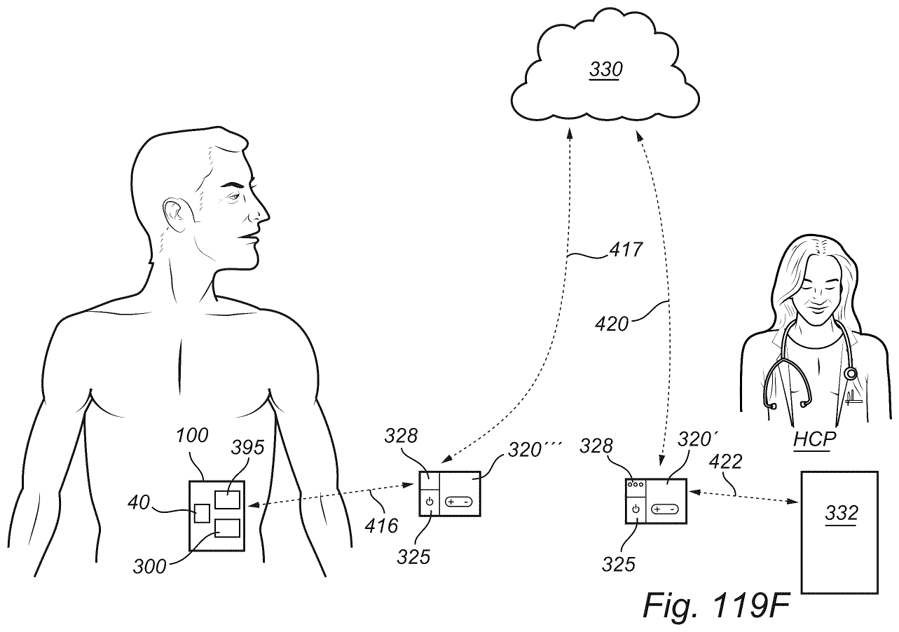

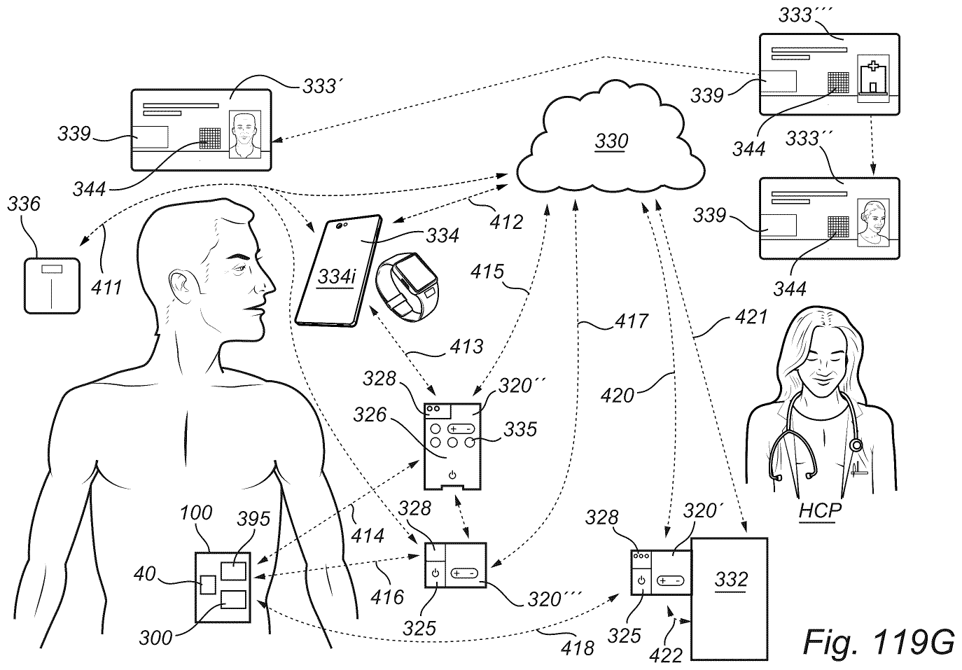

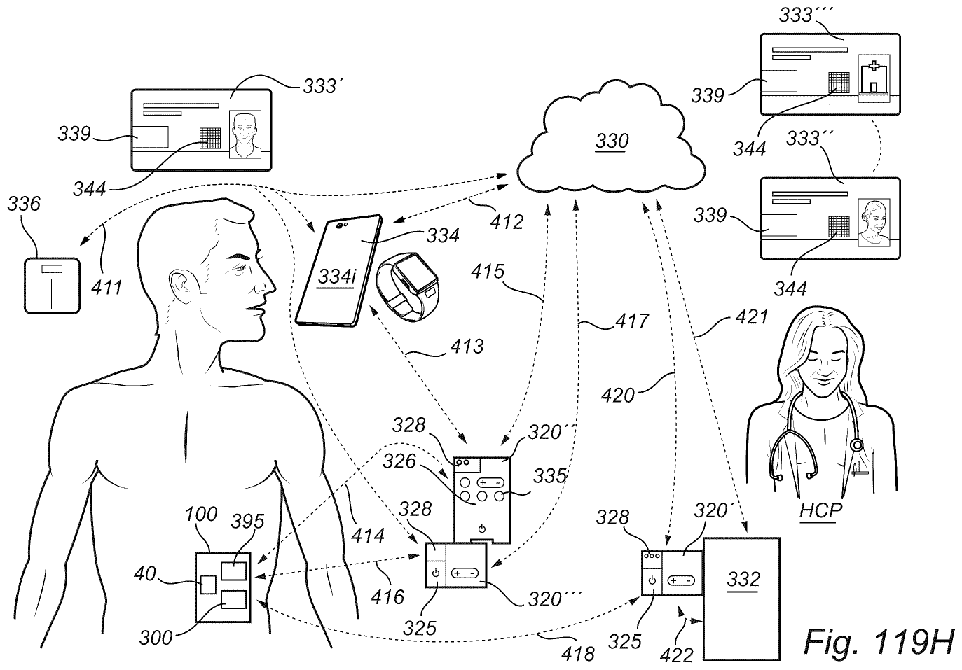

FIG. 119a and FIG. 119a′ shows one embodiment of a system for charging, programming and communicating with the controller 300 of the implanted medical device 100. FIGS. 119a and 119a′ further describes the communication and interaction between different external devices which may be devices held and operated by the patient, by the health care provider (HCP) or by the Dedicated Data Infrastructure (DDI), which is an infrastructure supplier for example by the manufacturer of the implanted medical device 100 or the external devices 320′, 320″, 320′″. The system of the embodiment of FIGS. 119a and 119a′ comprises three external devices 320′, 320″, 320′″ capable of communicating with the controller 300. The basic idea is to ensure the security of the communication with, and the operation of, the medical device 100 by having three external devices 320′, 320″, 320′″ with different levels of authority. The lowest level of authority is given to the patient operated remote control 320″.

The remote control external device 320″ is authorized to operate functions of the implanted medical device 100 via the implanted controller 300, on the basis of patient input. The remote control 320″ is further authorized to fetch some necessary data from the controller 300. The remote control 320″ is only capable of operating the controller 300 by communicating with the software currently running on the controller 300, with the currently settings of the software. The next level of authority is given to the Patient External Interrogation Device (P-EID) 320′″, which is a charging and communication unit which is held by the patient but is partially remotely operated by the Health Care Provider (HCP) (Usually a medical doctor with the clinic providing the treatment with help of the implanted medical device 100). The P-EID 320′″ is authorized to make setting changes by selecting pre-programmed steps of the software or hardware running on the controller 300 of the implanted medical device 100. The P-EID is remotely operated by the HCP, and receives input from the HCP, via the DDI. The highest level of authority is given to the HCP-EID 320′ and its controller the HCP Dedicated Display Device (DDD). The HCP-EID 320′ is a charging and communication unit which is held by the HCP physically at the clinic of the HCP. The HCP-EID 320′ is authorized to freely alter or replace the software running on the controller 300, when the patient is physically in the clinic of the HCP. The HCP-EID 320′ is controlled by the HCP DDD, which either acts on a “webview” portal from the HCP-EID or is a device closed down to any activities (which may include the absence of an internet connection) other than controlling and communicating with the HCP-EID. The webview portal does not necessarily mean internet based or HTML-protocol and the webview portal may be communicated over other communicating protocols such as Bluetooth or any other type of standard or proprietary protocol. The HCP DDD may also communicate with the HCP-EID over a local network or via Bluetooth or other standard or proprietary protocols.

Starting from the lowest level of authority, the patient remote control external device 320″ comprises a wireless transceiver 328 for communicating with the implanted medical device 100. The remote control 320″ is capable of controlling the operation of the implanted medical device 100 via the controller 300, by controlling pre-set functions of the implantable medical device 100, e.g. for operating an active portion of the implanted medical device 100 for performing the intended function of the implanted medical device 100. The remote control 320″ is able communicate with implanted medical device 100 using any standard or proprietary protocol designed for the purpose. In the embodiment shown in FIGS. 119a and 119a′, the wireless transceiver 328 comprises a Bluetooth (BT) transceiver, and the remote control 320″ is configured to communicate with implanted medical device 100 using BT. In an alternative configuration, the remote control 320″ communicates with the implanted medical device 100 using a combination of Ultra-Wide Band (UWB) wireless communication and BT. The use of UWB technology enables positioning of the remote control 320″ which can be used by the implanted medical device 100 as a way to establish that the remote control 320″ is at a position which the implanted medical device 100 and/or the patient can acknowledge as being correct, e.g. in the direct proximity to the medical device 100 and/or the patient, such as within reach of the patient and/or within 1 or 2 meters of the implanted medical device 100.

UWB communication is performed by the generation of radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. A UWB radio system can be used to determine the “time of flight” of the transmission at various frequencies. This helps overcome multipath propagation, since some of the frequencies have a line-of-sight trajectory, while other indirect paths have longer delay. With a cooperative symmetric two-way metering technique, distances can be measured to high resolution and accuracy. UWB is useful for real-time location systems, and its precision capabilities and low power make it well-suited for radio-frequency-sensitive environments, such as health care environments.

In embodiments in which a combination of BT and UWB technology is used, the UWB technology may be used for location-based authentication of the remote control 320″, whereas the communication and/or data transfer could take place using BT or any other way of communicating different from the UWB. The UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The remote control 320″ comprises computing unit 326 which runs a software application for communicating with the implanted medical device 100. The computing unit 326 can receive input directly from control buttons 335 arranged on the remote control 320″ or may receive input from a control interface 334i displayed on a patient display device 334 operated by the patient. In the embodiments in which the remote control 320″ receives input from a control interface 334i displayed on the patient display device 334 operated by the patient, the remote control 320″ transmits the control interface 334i in the form of a web-view portal, i.e. a remote interface that run in a sandbox environment on the patient's display device 334. A sandbox environment means that it runs on the display device 334 but only displays what is presented from the remote control and can only use a tightly controlled set of commands and resources, such as storage and memory space as well as network access, the ability to inspect the host system and read or write from other input devices connected to the display device 334 is extremely limited. Any action or command generated by the patient display device is like controlling a webpage. All acting software is located on the remote control that only displays its control interface onto the patient display unit. The computing unit 326 is further configured to encrypt the control interface before transmission to the patient display device 334, and encrypt the control commands before transmission to the implanted medical device 100. The computing unit 326 is further configured to transform the received user input into control commands for wireless transmission to the implantable medical device 100.

The patient's display device 334 could for example be a mobile phone, a tablet or a smart watch.

In the embodiment shown in FIGS. 119a and 119a′, the patient's display device 334 communicates with the remote control 320″ by means of BT. The control interface 334i in the form of a web-view portal is transmitted from the remote control 320″ to the patient's display device 334 over BT. Control commands in the form of inputs from the patient to the control interface 334i is transmitted from the patient's display device 334 to the remote control 320″, providing input to the remote control 320″ equivalent to the input that may be provided using the control buttons 335. The control commands created in the patient's display device 334 is encrypted in the patient's display device 334 and transmitted to the remote control 320′ using BT or any other communication protocol.

The remote control is normally not connected to the DDI or the Internet to increase security. In addition, the remote control 320″ may in one embodiment have its own private key and in a specific embodiment the remote control 320″ is activated by the patient's private key for a certain time period.

This may activate the function of the patient's display device and the remote wed-view display portal supplied by the remote control to the patient's display device.

The patient's private key is supplied in a patient private key device compromising a smartcard that may be inserted or provided close to the remote control 320″ to activate a permission to communicate with the implant 100 for a certain time period.

The patient's display device 334 may (in the case of the display device 334 being a mobile phone or tablet) comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the remote control 320″. Disconnecting the auxiliary radio connections reduces the risk that the integrity of the control interface 334i displayed on the patient's display device 334 is compromised, or that the control interface 334i displayed on the patient's display device 334 is remote controlled by an unauthorized device.

In alternative embodiments, control commands are generated and encrypted by the patient's display device and transmitted to the DDI 330. The DDI 330 could either alter the created control commands to commands readable by the remote control 320″ before further encrypting the control commands for transmission to the remote control 320″ or could simply add an extra layer of encryption before transmitting the control commands to the remote control 320″, or could simply act as a router for relaying the control commands from the patients' display device 334 to the remote control 320″. It is also conceivable that the DDI 330 adds a layer of end-to-end encryption directed at the implanted medical device 100, such that only the implanted medical device 100 can decrypt the control commands to perform the commands intended by the patient. In the embodiments above, when the patient remote display device 334 is communicating with the DDI, the patient's display device 334 may be configured to only display and interact with a web-view portal provided by a section of the DDI and it is conceivable that the web-view portal is a view of a back-end provided on the DDI 330, and in such embodiments, the patient interacting with the control interface on the patient's display device 334 is equivalent to the patient interacting with an area of the DDI 330.

The patient's display device 334 could have a first and second application related to the implanted medical device 100. The first application is the control application displaying the control interface 334i for control of the implanted medical device 100, whereas the second application is a general application for providing the patient with general information of the status of the implanted medical device 100 or information from the DDI 330 or HCP, or for providing an interface for the patient to provide general input to the DDI 330 or HCP related to the general wellbeing of the patient, the lifestyle of the patient or related to general input from the patient concerning the function of the implanted medical device 100. The second application, which do not provide input to the remote control 320″ and/or the implanted medical device 100 thus handles data which is less sensitive. As such, the general application could be configured to function also when all auxiliary radio connections are activated, whereas switching to the control application which handles the more sensitive control commands and communication with the implanted medical device 100 could require that the auxiliary radio connections are temporarily de-activated. It is also conceivable that the control application is a sub-application running within the general application, in which case the activation of the control application as a sub-application in the general application could require the temporary de-activation of auxiliary radio connections. In the embodiment shown in FIGS. 119a and 119a′, access to the control application requires the use of the optical and/or NFC means of the hardware key 333′ in combination with biometric input to the patient's display device, whereas accessing the general application only requires biometric input to the patient's display device and/or a pin code. In the alternative, a two-factor authentication solution, such as a digital key in combination with a pin code could be used for accessing the general application and/or the control application.

In general, a hardware key is needed to activate the patient display device 334 for certain time period to control the web-view portal of the remote control 320″, displaying the control interface 334i for control of the implanted medical device 100.

In the embodiments in which the patients display device 334 is configured to only display and interact with a web-view provided by another unit in the system, it is conceivable that the web-view portal is a view of a back-end provided on the DDI 330, and in such embodiments, the patient interacting with the control interface on the patient's display device is equivalent to the patient interacting with an area of the DDI 330.

Moving now to the P-EID 320′″. The P-EID 320′″ is an external device used by the patient, patient external device, which communicates with, and charges, the implanted medical device 100. The P-EID 320′″ can be remotely controlled by the HCP to read information from the implanted medical device 100. The P-EID 320′″ controls the operation of the implanted medical device 100, control the charging of the medical device 100, and adjusts the settings on the controller 300 of the implanted medical device 100 by changing pre-defined pre-programed steps and/or by the selection of pre-defined parameters within a defined range, e.g. Just as the remote control 320″, the P-EID 320′″ could be configured to communicate with the implanted medical device 100 using BT or UWB communication or any other proprietary or standard communication method. Since the device may be used for charging the implant, the charging signal and communication could be combined. Just as with the remote control 320″, it is also conceivable to use a combination of UWB wireless communication and BT for enabling positioning of the P-EID 320″ as a way to establish that the P-EID 320″ is at a position which the implanted medical device 100 and/or patient and/or HCP can acknowledge as being correct, e.g. in the direct proximity to the correct patient and/or the correct medical device 100. Just as for the remote control 320″, in embodiments in which a combination of BT and UWB technology is used, the UWB technology may be used for location-based authentication of the P-EID 320″, whereas the communication and/or data transfer could take place using BT. The P-EID 320″ comprises a wireless transmitter/transceiver 328 for communication and also comprises a wireless transmitter 325 configured for transferring energy wirelessly, which may be in the form of a magnetic field or any other signal such as electromagnetic, radio, light, sound or any other type of signal to transfer energy wirelessly to a wireless receiver 395 of the implanted medical device 100. The wireless receiver 395 of the implanted medical device 100 is configured to receive the energy in the form of the magnetic field and transform the energy into electric energy for storage in an implanted energy storage unit 40, and/or for consumption in an energy consuming part of the implanted medical device 100 (such as the operation device, controller 300 etc.). The magnetic field generated in the P-EID 320′″ and received in the implanted medical device 100 is denoted charging signal. In addition to enabling the wireless transfer of energy from the P-EID 320′″ to the implanted medical implant 100, the charging signal may also function as a means of communication. E.g., variations in the frequency of the transmission, and/or the amplitude of the signal may be uses as signaling means for enabling communication in one direction, from the P-EID 320′″ to the implanted medical device 100, or in both directions between the P-EID 320′″ and the implanted medical device 100. The charging signal in the embodiment shown in FIGS. 119a and 119a′ is a signal in the range 10 65 kHz or 115-140 kHz and the communication follow a proprietary communication signaling protocol, i.e., it is not based on an open standard. In alternative embodiments, BT could be combined with communication using the charging signal, or communication using the charging signal could be combined with an UWB signal. The energy signal could also be used as a carrying signal for the communication signal.

Just as for the remote control 320″, the UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In the alternative, the charging signal could be used as a wakeup signal for the BT, as the charging signal does not travel very far. Also, as a means of location-based authentication, the effect of the charging signal or the RSSI could be assessed by the controller 300 in the implanted medical device 100 to establish that the transmitter is within a defined range. In the BT/UWB combination, the UWB may be used also for transmission of data. In some embodiments, the UWB and/or the charging signal could be used for the transmission of some portions of the data, such as sensitive portions of the data, or for the transmission keys for unlocking encrypted communication sent by BT. Wake-up could be performed with any other signal.

UWB could also be used for waking up the charging signal transmission, to start the wireless transfer of energy or for initiating communication using the charging signal. As the signal for transferring energy has a very high effect in relation to normal radio communication signals, the signal for transferring energy cannot be active all the time, as this signal may be hazardous e.g., by generating heat.

The P-EID 320′″ communicates with the HCP over the Internet by means of a secure communication, such as over a VPN. The communication between the HCP and the P-EID 320′″ is preferably encrypted. Preferably, the communication is sent via the DDI, which may only be relying the information. The communication from the HCP to the implanted medical device 100 may be performed using an end-to-end encryption, in which case the communication cannot be decrypted by the P-EID 320′″. In such embodiments, the P-EID 320′″ acts as a router, only passing on encrypted communication from the HCP to the controller 300 of the implanted medical device 100 (without full decryption). This solution further increases security as the keys for decrypting the information rests only with the HCP and with the implanted medical device 100, which reduces the risk that an unencrypted signal is intercepted by an unauthorized device. The P-EID 320′″ may add own encryption or information, specifically for security reasons. The P-EID 320′″ may hold its own private key and may be allowed to communicate with the implant 100 based on confirmation from the patient's private key, which may be provided as a smartcard to be inserted in a slot of the P-EID 320′″ or hold in close proximity thereto to be read by the P-EID 320′″. These two keys will add a high level of security to the performed communication between the Implant 100 and the P-EID 320′″ since the patient's hardware key in this example on the smartcard may activate and thereby allow the communication and action taken in relation to the implant. The P-EID 320′″ may as previously described change the treatment setting of the implant by selecting pre-programmed steps of the treatment possibilities. Such pre-programmed treatment options may include for example to change:
at least one of the position, frequency and level of compression of an implanted heart compression device,
the flow of an apparatus assisting the pump function of a heart of the patient,
the flow of an apparatus assisting the pump function comprising a turbine bump placed within a patient's blood vessel for assisting the pump function of the heart,
the function of an operable artificial heart valve,
at least one of the function of, the valve opening pressure and time for closure of an operable artificial heart valve for increasing the blood flow to the coronary arteries.
at least one of the functions of, the amount and/or concentration of a drug from an implantable drug delivery device,
at least one of the injection site and frequency as well as amount of drug delivered by an implantable drug delivery device for injecting directly into a blood vessel and change the position of the injection site, all from within the patient's body,
at least one of the injection site and frequency as well as amount of drug delivered by an implantable drug delivery device for injecting potency enhancing drugs into an erectile tissue of the patient,
at least one of the level of constriction, pressure or position of a hydraulic, mechanic, and/or electric constriction implant,
the volume of an operable volume filling device,
the constriction of an operable gastric band,
at least one of the level and time of stretching and when such stretching occur in relation to food intake of a patient for an operable implant for stretching the stomach wall of the patient for creating satiety,
when an action should be taken relating to an implant configured to sense the amount of food intake based on number of times a patient swallows solid food,
at least one of the size and shape of an operable cosmetic implant,
at least one of the shape and size in the breast region of a patient of an operable cosmetic implant for adjustment,
at least one of pressure, volume, sensor input or time of an implant controlling medical device for the emptying of a urinary bladder,
at least one of the closing pressure, the time to close after urinating, how much extra pressure would be allowed at exercise of an implant hindering urinary leakage,
at least one of the closing pressure, the time to close after revealing, how much extra pressure would be allowed at exercise of an implant hindering anal incontinence,
parameters of an implant controlling the emptying of fecal matter, such as pressure, volume, pump or motor position etc.,
parameters of an implant monitoring an aneurysm, such as pressure, aneurysm expansion, volume, reservoir volume, etc.,
parameters of an implant for hindering the expansion of an aneurysm, such as pressure, aneurysm expansion, volume, reservoir volume, etc.,
parameters of an implant lubricating a joint, such as volume, reservoir volume, etc.,
parameters of an implant for affecting the blood flow to an erectile tissue of the patient, such as the level of constriction, pressure or position of a hydraulic, mechanic, and/or electric constriction implant,
parameters of an implant for simulating the engorgement of an erectile tissue, such as the level of stimulation, frequency, or amplitude of an electrical stimulation,
parameters of an implant with a reservoir for holding bodily fluids, such as volume, reservoir volume, etc.,
parameters of an implant storing and/or emptying a bodily reservoir or a surgically created reservoir, such as stimulation parameters in a peristaltic wave, stretch or bending sensors, reservoir volume, etc.,
parameters of an implant communicating with a database outside the body, such as key handshake, new key pairing, signal amplitude etc.,
parameters of an implant able to be programmed from outside the body,
parameters of an implant able to be programmed from outside the body with a wireless signal,
parameters of an implant treating impotence, such as pressure, amount of drug delivered, time for erection period etc.,
parameters of an implant controlling the flow of eggs in the uterine tube, such as the level of constriction, time period, position of a hydraulic, mechanic, and/or electric constriction implant,
parameters of an implant controlling the flow of sperms in the uterine tube, such as the level of stimulation, frequency, or amplitude of an electrical stimulation,
parameters of an implant controlling the flow of sperms in the vas deferens, such as the level of constriction, time period, position of a hydraulic, mechanic, and/or electric constriction implant,
parameters of an implant for hindering the transportation of the sperm in the vas deferens, such as the level of constriction, time period, position of a hydraulic, mechanic, and/or electric constriction implant,
parameters of an implant treating osteoarthritis,
parameters of an implant performing a test of parameters inside the body,
parameters of an implant controlling specific treatment parameters from inside the body,
parameters of an implant controlling bodily parameters from inside the body,
parameters of an implant controlling the blood pressure,
parameters of an implant controlling the blood pressure by affecting the dilatation of the renal artery, such as heat and time period in relation to blood pressure,
parameters of an implant controlling a drug treatment parameter,
parameters of an implant controlling a parameter in the blood,
parameters of an implant for adjusting or replacing any bone part of a body of the patient,
parameters of an implant replacing an organ of the patient or part of an organ of the patient or the function thereof, parameters of a vascular treatment device, such as bending, expanding sensor,
parameters of an implant adapted to move fluid inside the body of the patient, such as volume, pumping parameters,
parameters of an implant configured to sense a parameter related to the patient swallowing,
parameters of an implant configured to exercise a muscle with electrical or mechanical stimulation, such as stimulation parameters, amplitude frequency time period etc.,
parameters of an implant configured for emptying an intestine portion on command, such as electrical stimulation parameters, peristaltic wave adjustment etc., parameters of an operable implant configured to be invaginated in the stomach of the patient to reduce the volume of the stomach substantially more than the volume of the device, such as volume,
parameters of an implant configured for emptying the urinary bladder from within the patient's body by compressing the bladder, such as pressure, volume and time parameters of an implant configured for draining fluid from within the patient's body,
parameters of an implant configured for the active lubrication of a joint with an added lubrication fluid, such as frequency and/or volume of the drug supplied,
parameters of an implant configured for removing clots and particles from the patient's blood stream,
parameters of an implant configured for elongating or straightening a bone in the patient, to reduce scoliosis, force, length etc., parameters of a device to stimulate the brain for a several position to a focused point, parameters of an artificial stomach replacing the function of the natural stomach,
parameters of an implant configured for adjusting the position of a female's urinary tract or bladder neck,
parameters of an implant configured for stimulating the ampulla vas deference and creating temporary constriction.

When the implanted medical device 100 is to be controlled and/or updated remotely by the HCP, via the P-EID 320′″, a HCP Dedicated Device (DD) 332 displays an interface in which predefined program steps or setting values are presented to the HCP. The HCP provides input to the HCP DD 332 by selecting program steps, altering settings and/or values or by altering the order in which pre-defined program steps is to be executed. The instructions/parameters inputted into the HCP DD 332 for remote operation is in the embodiment shown in FIGS. 119a and 119a′ routed to the P-EID 320′″ via the DDI 330, which may or may not be able to decrypt/read the instructions. The DDI 330 may store the instructions for a time period to later transfer the instructions in a package of created instructions to the P-EID 320′″.

It is also conceivable that an additional layer of encryption is provided to the package by the DDI 330. The additional layer of encryption may be a layer of encryption to be decrypted by the P-EID 330, or a layer of encryption which may only be decrypted by the controller 300 of the implanted medical device 100, which reduces the risk that unencrypted instructions or packages are intercepted by unauthorized devices. The instructions/parameters are then provided to the P-EID 320″, which then loads the instructions/parameters into the during the next charging/energy transfer to the implanted medical device 100 using any of the signal transferring means (wireless or conductive) disclosed herein.

The Health Care Provider EID (HCP EID) 320′ have the same features as the P-EID 320″ and can communicate with the implanted medical device 100 in the same alternative ways (and combinations of alternative ways) as the P-EID 320′″. However, in addition, the HCP EID 320′ also enables the HCP to freely reprogram the controller 300 of the implanted medical device 100, including replacing the entire program code running in the controller 300. The idea is that the HCP EID 320′ always remain with the HCP and as such, all updates to the program code or retrieval of data from the implanted medical device 100 using the HCP EID 320′ is performed with the HCP and patient present (i.e. not remote). The physical presence of the HCP is an additional layer of security for these updates which may be critical to the function of the implanted medical device 100.

In the embodiment shown in FIGS. 119a and 119a′, the HCP communicates with the HCP EID 320′ using a HCP Dedicated Display Device 332 (HCP DDD), which is a HCP display device comprising a control interface for controlling and communicating with the HCP EID 320′. As the HCP EID 320′ always stays physically at the HCP's clinic, communication between the HCP EID 320′ and HCP DDD 332 does not have to be sent over the Internet. Instead, the HCP DDD 332 and the HCP EID 320′ can communicate using one or more of BT, a proprietary wireless communication channel, or a wired connection. The alteration to the programming is then sent to the implanted medical device 100 directly via the HCP EID 320′. Inputting into the HCP DDD 332 for direct operation by means of the HCP EID 320′ is the same as inputting directly into the HCP EID 320′, which then directly transfers the instructions into the implanted medical device 100.

In the embodiment shown in FIGS. 119a and 119a′, both the patient and the HCP has a combined hardware key 333′, 333″. The combined keys 333′, 333″ comprises a hardware component comprising a unique circuitry (providing the highest level of security), a wireless NFC-transmitter 339 for transmitting a specific code (providing mid-level security), and a printed QR-code 344 for optical recognition of the card (providing the lowest level of security). The HCP private key is supplied by a HCP private key device 333″ adapted to be provided to the HCP EID external device via at least one of; a reading slot or comparable for the HCP private key device 333″, an RFID communication or other close distance wireless activation communication to both the HCP EID 320′ and the HCP DDD 332 if used. The HCP DDD 332 will be activated by such HCP private key device 333″, which for example may comprise at least one of, a smartcard, a key-ring device, a watch an arm or wrist band a neckless or any shape device.

The HCP EID external device may comprise at least one of;
a reading slot or comparable for the HCP private key device,
an RFID communication and
other close distance wireless activation communication means

The HCP external device 320′ may further comprise at least one wireless transceiver 328 configured for communication with a data infrastructure server, DDI, through a first network protocol.

A dedicated data infrastructure server, DDI, is in one embodiment adapted to receive commands from said HCP external device 320′ and may be adapted to rely the received commands without opening said commands directed to the patient external device 320″, the DDI 330 comprising one wireless transceiver configured for communication with said patient external device 320″.

The patient EID external device 320″ is in one embodiment adapted to receive the commands relayed by the DDI, and further adapted to send these commands to the implanted medical device 100, which is adapted to receive commands from the HCP, Health Care Provider, via the DDI 330 to change the pre-programmed treatment steps of the implanted medical device 100. The patient EID is adapted to be activated and authenticated and allowed to perform the commands by the patient providing a patient private key device 333′. The patient's private key device is in one embodiment adapted to be provided to the patient external device by the patient via at least one of; a reading slot or comparable for the patient private key device 333′, an RFID communication or other close distance wireless activation communication.

The patient EID external device, in one or more embodiments, comprises at least one of;
a reading slot or comparable for the HCP private key device,
an RFID communication, or
other close distance wireless activation communication

The patient EID external device may in one or more embodiments comprise at least one wireless transceiver configured for communication with the implanted medical device through a second network protocol.

The patient's key 333′ is in the embodiment shown in FIGS. 119a and 119a′ in the form of a key card having an interface for communicating with the P-EID 320′″, such that the key card could be inserted into a key card slot in the P-EID 320″. The NFC-transmitter 339 and/or the printed QR-code 344 can be used as means for accessing the control interface 334i of the display device 334. In addition, the display device 334 may require a pin-code and/or a biometric input, such as face recognition or fingerprint recognition.

The HCP's key 333″, in the embodiment shown in FIGS. 119a and 119a′ is in the form of a key card having an interface for communicating with the HCP-EID 320′, such that in one embodiment the key card could be inserted into a key card slot in the HCP-EID 320′. The NFC-transmitter 339 and/or the printed QR-code 344 can be used as means for accessing the control interface of the HCP DDD 332. In addition, the HCP DDD 332 may require a pin-code and/or a biometric input, such as face recognition or fingerprint recognition.

In alternative embodiments, it is however conceivable that the hardware key solution is replaced by a two-factor authentication solution, such as a digital key in combination with a PIN code or a biometric input (such as face recognition and/or fingerprint recognition). The key could also be a software key, holding similar advance key features, such as the Swedish Bank ID being a good example thereof.

In the embodiment shown in FIGS. 119a and 119a′, communication over the Internet takes place over a Dedicated Data Infrastructure (DDI) 330, running on a cloud service. The DDI 330 in this case handles communication between the HCP DDD 332 and the P-EID 320′″. however, the more likely scenario is that the HCP DDD 332 is closed down, such that only the necessary functions of the control application can function on the HCP DDD 332. In the closed down embodiment, the HCP DDD 332 is only able to give the necessary commands to HCP EID 320′ to further update the pre-programmed treatment steps of the Implant 100 via the P-EID 320′″ in direct contact, or more likely indirect contact via the DDI 332. If the patient is present locally, the HCP EID may communicate and act directly on the patient's implant. However, before anything is accepted by the implant, a patient private key device 333′ has to be presented to the P EID 320′″ or HCP EID 320′ for maximum security.

The DDI 330 is logging information of the contact between the HCP and the remote control 320″ via implant feedback data supplied from the implant to P-EID 320′″. Data generated between the HCP and the patient's display device 334, as well as between the HCP and auxiliary devices 336 (such as tools for following up the patient's treatments e.g. a scale in obesity treatment example or a blood pressure monitor in a blood pressure treatment example) are logged by the DDI 330. In some embodiments, although less likely, the HCP DDD 332 may also handle the communication between the patient's display device 334 and the remote control 320″. In FIG. 119a′, the auxiliary devices 336 is connected to the P-EID as well and can thus provide input from the auxiliary devices 336 to the P-EID which can be used by the P-EID for altering the treatment or for follow up.

In all examples, the communication from the HCP to: the P-EID 320′″, the remote control 320″, the patient's display device 334 and the auxiliary devices 336 may be performed using an end-to-end encryption. In embodiments with end-to-end encryption, the communication cannot be decrypted by the DDI 330. In such embodiments, the DDI 330 acts as a router, only passing on encrypted communication from the HCP to various devices. This solution further increases security as the keys for decrypting the information rests only with the HCP and with the device sending or receiving the communication, which reduces the risk that an unencrypted signal is intercepted by an unauthorized device. The P-EID 320′″ may also only pass on encrypted information.

In addition to acting as an intermediary or router for communication, the DDI 330 collects data on the implanted medical device 100, on the treatment and on the patient. The data may be collected in an encrypted form, in an anonymized form or in an open form. The form of the collected data may depend on the sensitivity of the data or on the source from which the data is collected. In the embodiment shown in FIGS. 119a and 119a′, the DDI 330 sends a questionnaire to the patient's display device 334. The questionnaire could comprise questions to the patient related to the general health of the patient, related to the way of life of the patient, or related specifically to the treatment provided by the implanted medical device 100 (such as for example a visual analogue scale for measuring pain). The DDI 330 could compile and/or combine input from several sources and communicate the input to the HCP which could use the provided information to create instructions to the various devices to be sent back over the DDI 330. The data collection performed by the DDI 330 could also be in the form a log to make sure that all communication between the units in the system can be back traced. Logging the communication ensures that all alterations to software or the settings of the software, as well as the frequency and operation of the implanted medical device 100 can be followed. Following the communication enables the DDI 330 or the HCP to follow the treatment and react it something in the communication indicates that the treatment does not provide the intended results or if something appears to be wrong with any of the components in the system. If patient feedback from the patient display device 334 indicates that a new treatment step of the implant is needed, such information must be confirmed by direct contact between HCP and patient.

In the specific embodiment disclosed in FIGS. 119a and 119a′, the wireless connections between the different units are as follows. The wireless connection 411 between the auxiliary device 336 and the DDI 330 is based on Wi-Fi or a mobile telecommunication regime or may be sent to the DDI 330 via the P-EID 320′″ and the wireless connection 411 between the auxiliary device 336 and the patient's display device 334 is based on BT or any other communication pathway disclosed herein. The wireless connection 412 between the patient's display device 334 and the DDI 330 is based on Wi-Fi or a mobile telecommunication regime. The wireless connection 413 between the patient's display device 334 and the remote control 320″ is based on BT or any other communication pathway disclosed herein. The wireless connection 414 between the patient remote control 320″ and the implanted medical device 100 is based on BT and UWB or any other communication pathway disclosed herein. The wireless connection 415 between the remote control 320″ and the DDI 330 is likely to not be used, and if present be based on Wi-Fi or a mobile telecommunication regime. The wireless connection 416 between the P-EID 320′″ and the implanted medical device 100 is based on BT, UWB and the charging signal or any other communication or energizing pathway disclosed herein. The wireless connection 417 between the P-EID 320′″ and the DDI 330 is based on Wi-Fi or a mobile telecommunication regime. The wireless connection 418 between the HCP-EID 320′ and the implanted medical device 100 is based on at least one of the BT, UWB and the charging signal. The wireless connection 419 between the P-EID 320′″ and the HCP DD 332 is based on BT or any other communication path disclosed herein. The wireless connection 420 between the HPC-EID 320′ and the DDI 330 is based on Wi-Fi or a mobile telecommunication regime. The wireless connection 421 between the HPC DD 332 and the DDI 330 is normally closed and not used and if so based on Wi-Fi or a mobile telecommunication regime. The wireless connection 422 between the HCP-EID 320′ and the HCP DD 332 is based on at least one of BT, UWB, local network or any other communication path disclosed herein.

The wireless connections specifically described in the embodiment shown in FIGS. 119a and 119a′ may however be replaced or assisted by wireless connections based on radio frequency identification (RFID), near field communication (NFC), Bluetooth, Bluetooth low energy (BLE), or wireless local area network (WLAN). The mobile telecommunication regimes may for example be 1G, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

FIG. 119a′ also discloses a master private key 333′″ device that allow issuance of new private key device wherein the HCP or HCP admin have such master private key 333′″ device adapted to be able to replace and pair a new patient private key 333′ device or HCP private key device 333″ into the system, through the HCP EID external device 320′.

A system configured for changing pre-programmed treatment settings of an implantable medical device, when implanted in a patient, from a distant remote location in relation to the patient, the system comprising:

FIG. 119a′ also discloses a scenario in which at least one health care provider, HCP, external device 320′ is adapted to receive a command from the HCP to change said pre-programmed treatment settings of an implanted medical device 100, further adapted to be activated and authenticated and allowed to perform said command by the HCP providing a HCP private key device 333″. The HCP EID external device 320′ further comprising at least one wireless transceiver 328 configured for communication with a patient EID external device 320′″, through a first network protocol. The system comprises the patient EID external device 320′″, the patient EID external 320′″ device being adapted to receive command from said HCP external device 320′, and to relay the received command without modifying said command to the implanted medical device 100. The patient EID external device 320′″ comprising one wireless transceiver 328. The patient EID 320′″ is adapted to send the command to the implanted medical device 100, to receive a command from the HCP to change said pre-programmed treatment settings of the implanted medical device 100, and further to be activated and authenticated and allowed to perform said command by the patient providing a patient private key 333′ device comprising a patient private key.

Although wireless transfer is primarily described in the embodiment disclosed with reference to FIGS. 119a, 119a′ the wireless communication between any of the external device may be substituted for wired communication. Also, some or all of the wireless communication between an external device and the implanted medical device 100 may be substituted for conductive communication using a portion of the human body as conductor.

FIG. 119b shows a portion of FIG. 119a, in which some of the components have been omitted to outline a specific scenario. In the scenario outlined in FIG. 119b, the system is configured for changing pre-programmed treatment settings of an implantable medical device 100, when implanted in a patient, from a distant remote location in relation to the patient. The system if FIG. 119b comprises at least one HCP EID 320′ external device adapted to receive commands from the HCP to change said pre-programmed treatment settings of an implanted medical device 100. The HCP EID 320′ external device is further adapted to be activated and authenticated and allowed to perform said command by the HCP providing a HCP private key device 333″ adapted to be provided to the HCP EID external device 320′.

The private key device 333″ is adapted to be provided to the HCP EID external device 320′ via at least one of: a reading slot or comparable for the HCP private key device 333″, and an RFID communication or other close distance wireless activation communication. The HCP EID external device 320′ comprises at least one of: a reading slot or comparable for the HCP private key device 333″, an RFID communication, and other close distance wireless activation communication or electrical direct contact.

The HCP EID external device 320′ further comprises at least one wireless transceiver 328 configured for communication with a dedicated data infrastructure server (DDI) 330, through a first network protocol.

The system further comprises a dedicated data infrastructure server (DDI) 330, adapted to receive command from said HCP EID external device 320′, adapted to relay the received commands without modifying said command to a patient EID external device 320′″. The dedicated data infrastructure server (DDI) 330 further comprises a wireless transceiver 328 configured for communication with said patient external device. The system further comprises a patient EID external device 320′″ adapted to receive the command relayed by the dedicated data infrastructure server (DDI) 330 and further adapted to send commands to the implanted medical device 100 and further adapted to receive commands from the HCP EID external device 320′ via the dedicated data infrastructure server (DDI) 330 to change said pre-programmed treatment settings of the implanted medical device 100. The patient EID external device 320′″, and further adapted to be activated and authenticated and allowed to perform said command by the patient providing a patient private key device 333′ adapted to be provided to the patient EID external device 320′″ by the patient via at least one of: a reading slot or comparable for the patient private key device 333′, an RFID communication or other close distance wireless activation communication or electrical direct contact. The patient EID external device 320′″ further comprises at least one of: a reading slot or comparable for the HCP private key device, an RFID communication and other close distance wireless activation communication or electrical direct contact. The patient EID external device 320′″ further comprises at least one wireless transceiver 328 configured for communication with the implanted medical device 100 through a second network protocol. The implanted medical device 100 is in turn configured to treat the patient or perform a bodily function.

The scenario described with reference to FIG. 119b may in alternative embodiments be complemented with additional units or communication connections, or combined with any of the scenarios described with reference to FIGS. 119c-119e.

FIG. 119c shows a portion of FIG. 119a, in which some of the components have been omitted to outline a specific scenario. In the scenario outlined in FIG. 119c, system configured for changing pre-programmed treatment settings of an implantable medical device 100 is disclosed. The changing pre-programmed treatment settings are performed by a health care provider (HCP) in the physical presence of the patient. The system comprises at least one HCP EID external device 320′ adapted to receive commands from the HCP, directly or indirectly, to change said pre-programmed treatment settings in steps of an implantable medical device 100, when implanted. The HCP EID external device 320′ is further adapted to be activated, authenticated, and allowed to perform said command by the HCP providing a HCP private key device 333″ comprising a HCP private key. The HCP private key device in the embodiment of FIG. 119c, comprises at least one of a smart card, a keyring device, a watch, an arm or wrist band, a necklace, and any shaped device. The HCP EID external device 320′ is adapted to be involved in at least one of receiving information from the implant 100, receiving information from a patient remote external device 336, actuating the implanted medical device 100, changing pre-programmed settings, and updating software of the implantable medical device 100, when implanted.

The HCP EID external device 320′ is adapted to be activated, authenticated, and allowed to perform said command also by the patient, the system comprises a patient private key device 333′ comprising a patient private key. The patient private key device 333′ comprising at least one of a smart card, a keyring device, a watch, an arm or wrist band, a necklace, and any shaped device. The HCP private key 333″ and the patients private key are required for performing said actions by the HCP EID external device 320′ to at least one of receive information from the implant 100, to receive information from a patient remote external device 336, to actuate the implanted medical device 100, to change pre-programmed settings, and to update software of the implantable medical device 100, when the implantable medical device is implanted.

FIG. 119c also outlines a scenario in which the system is configured for changing pre-programmed treatment settings in steps of an implantable medical device, when implanted in a patient, by a health care provider, HCP, with the patient on remote on distance, the system comprising: at least one HCP EID external device 320′ adapted to receive a command from the HCP direct or indirect, to change said pre-programmed treatment settings in steps of an implantable medical device, when implanted, wherein the HCP EID external device 320′ is further adapted to be activated, authenticated, and allowed to perform said command by the HCP. The said action by the HCP EID external device 320′ to change pre-programmed settings in the implant 100 and to update software of the implantable medical device 100, when the implantable medical device 100 is implanted, is adapted to be authenticated by a HCP private key device 333″ and a patient private key device 333′.

The scenario described with reference to FIG. 119c may in alternative embodiments be complemented with additional units or communication connections, or combined with any of the scenarios described with reference to FIG. 119b, or 119d-119e.

FIG. 119d shows a portion of FIG. 119a, in which some of the components have been omitted to outline a specific scenario. In the scenario outlined in FIG. 119d, a system configured to change pre-programmed and pre-selected treatment actions of an implantable medical device 100 by command from the patient is described. The system comprises an implantable medical device 100, a patient remote external device 320″, and a wireless transceiver 328 configured for communication with the implantable medical device 100, when the medical device is implanted, through a second network protocol, The system further comprises a remote display portal interface 334i configured to receive content delivered from the patient remote external device 320″ to expose buttons to express the will to actuate the functions of the implanted medical device 100 by the patient through the patient remote external device 320″. The remote external device 320″ is further configured to present the display portal remotely on a patient display device 334 allowing the patient to actuate the functions of the implanted medical device 100 through the display portal of the patient remote external device 320″ visualized on the patient display device 334. In FIG. 119d, a further wireless connection 423 between the patient remote external device 320″ and the patient EID external device 320′″ is provided. This further wireless connection 423 could be a wireless connection according to any one of the wireless signaling methods and protocols described herein, and the communication can be encrypted.

The scenario described with reference to FIG. 119d may in alternative embodiments be complemented with additional units or communication connections, or combined with any of the scenarios described with reference to FIG. 119b, 119c, or 119e.

FIG. 119e shows a portion of FIG. 119a, in which some of the components have been omitted to outline a specific scenario. In the scenario outlined in FIG. 119e, a system configured for providing information from an implantable medical device 100, when implanted in a patient, from a distant remote location in relation to the patient is described. The system comprises at least one patient EID external device 320′″ adapted to receive information from the implant 100, and adapted to send such information further on to a server or dedicated data infrastructure, DDI, 330. The patient EID external device 320′″ is further adapted to be activated and authenticated and allowed to receive said information from the implanted medical device 100 by the patient providing a private key, The patient private key device comprises the private key adapted to be provided to the patient EID external device 320′″ via at least one of; a reading slot or comparable for the patient private key device, an RFID communication or other close distance wireless activation communication or direct electrical connection, The patient EID external device 320′″ comprises at least one of; a reading slot or comparable for the patient private key device, an RFID communication and other close distance wireless activation communication or direct electrical contact, The patient EID external device 320′″ further comprises at least one wireless transceiver 328 configured for communication with the DDI 330, through a first network protocol.

The scenario described with reference to FIG. 119e may in alternative embodiments be complemented with additional units or communication connections, or combined with any of the scenarios described with reference to FIGS. 119b-119d.

FIG. 119f shows a portion of FIG. 119a, in which some of the components have been omitted to outline a specific scenario. In the scenario outlined in FIG. 119f a system configured for changing pre-programmed treatment settings in steps of an implantable medical device 100, when implanted in a patient, by a health care provider, HCP, either in the physical presence of the patient or remotely with the patient on distance is described. The system comprising at least one HCP EID external device 320′ adapted to receive a command directly or indirectly from the HCP to change said pre-programmed treatment settings in steps of the implantable medical device 100, when implanted, wherein the HCP EID external device 320′ is further adapted to be activated, authenticated, and allowed to perform said command by the HCP providing a HCP private key device comprising a HCP private key, comprising at least one of a smart card, a keyring device, a watch, an arm or wrist band, a necklace, and any shaped device. The system further comprises a patient private key device comprising a patient private key comprising at least one of a smart card, a keyring device, a watch, an arm or wrist band, a necklace, and any shaped device. Both the HCP and patient private key is required for performing said action by the HCP EID external device 320′ to change the pre-programmed settings in the implant 100 and to update software of the implantable medical device 100, when the implantable medical device 100 is implanted.

The patient private key is adapted to activate, be authenticated, and allowed to perform said command provided by the HCP, either via the HCP EID external device or when the action is performed remotely via a patient EID external device 320′. In the embodiment shown in FIG. 119f, the communication is routed over the DDI server 330.

The scenario described with reference to FIG. 119f may in alternative embodiments be complemented with additional units or communication connections, or combined with any of the scenarios described with reference to FIGS. 119b-119e.

FIG. 119g shows an overview of an embodiment of the system, similar to that described with reference to FIG. 119a′, the difference being that the HCP EID and the HCP DDD are combined into a single device.

FIG. 119h shows an overview of an embodiment of the system, similar to that described with reference to FIG. 119a′, the difference being that the HCP EID 320′″ and the HCP DDD 332 are combined into a single device and the P EID 320′″ and the patient remote control external device 320″ are combined into a single device.

One probable scenario/design of the communication system is for the purpose of changing pre-programmed treatment settings of an implantable medical device, when implanted in a patient, from a distant remote location in relation to the patient. The system comprises at least one health care provider, HCP, external device 320′ adapted to receive a command from the HCP to change said pre-programmed treatment settings of an implanted medical device, further adapted to be activated and authenticated and allowed to perform said command by the HCP providing a HCP private key device 333″ adapted to be provided to an HCP EID external device via at least one of; a reading slot or comparable for the HCP private key device, a RFID communication or other close distance wireless activation communication.

The HCP EID external device comprising at least one of a reading slot or comparable for the HCP private key device, a RFID communication, and other close distance wireless activation communication or electrical direct contact. The HCP EID external device further comprises at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device. The patient EID external device comprising one wireless transceiver configured for communication with said patient external device. The patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.

In another scenario, the implantable medical device may be configured to transmit information. Such information may, for example, relate to a function of the implantable medical device, a parameter of the body of the patient, measurements, among others. In that scenario, the implantable medical device may be configured to only transmit such data in response to a received authentication. The authentication may be received from the patient EID, or from another external device. The implantable medical device may verify that the authenticated device is authorized to request data, for example through a cryptographic verification, which in some examples is based on a key stored at the implantable medical device.

The patient EID (alternatively patient external device) may provide the authentication based on a patient private key provided to the patient EID. The implantable medical device may in that scenario verify that the authentication is based on a patient private key associated with a patient that is authorized to request information from the implant. Based on a valid authorization, the implantable medical device may send data to the patient external device. The data may in some examples be encrypted, for example in any of the ways of encrypting data from the medical implant are described herein. The authorization may be a one-time authorization, an authorization for a predetermined time interval or an authorization that is valid until withdrawn. For example, the authorization may be provided once a day, or at the time of requesting the data from the implantable medical device.

While FIGS. 65F and 65FA-FH and the description thereof discloses different scenarios, also other combinations of authentication or authorization are conceivable. For example, for changing settings of an implantable medical device, it may be required that a health care provider provides an authentication for the changes using their HCP private key. In some examples, an authorization is necessary also from the patient by the patient providing a patient private key device in order for the changes to be accepted by the implantable medical implant. In some examples, the patient may delegate its authorization to the health care provider by using the patient private key.

In some cases, when both an authorization from the health care provider and the patient are required, the health care provider and the patient could be in the same location. To provide an authorization showing that both the health care provider and the patient are at the same location, either the HCP external device or the patient external device may be adapted to receive both the patient private key and the HCP private key in order to authorize a command or a change for the implantable medical implant. Alternatively, the HCP external device or the patient external device may be configured to communicate via a short range communications technology to verify that the other device is present and authenticated before sending the changes to the implantable medical device. This added security may be beneficial, for example, when the medical implant is re-programmed, or software of the implantable medical device is otherwise changed.

In other examples, both an authorization from a patient and from a health care provider may be required, but without the requirement that they are at the same location. In those examples, the authorization may be given using their respective external device. This may be beneficial, for example, when making changes to treatment settings or updating a software is considered to be low risk. Different programs comprised in the implantable medical implant may be considered to have a different risk level associated with them. A risk determination may be programmed into the implantable medical implant as conditions for accepting an update. If the implantable medical implant determines that an update fulfils the conditions, it may install it, otherwise, if the implantable medical implant determines that the conditions are not fulfilled, it may reject the update.

In some examples, it may be sufficient to only require an authorization from at least one of a health care provider and a patient. For example, changes associated with a lower risk, such as changing pre-programmed settings or treatment settings within pre-determined ranges, may be performed using only one authorization.

Although the different scenarios outlined in FIGS. 119b-119g are described with specific units and method of signaling, these scenarios may very well be combined with each other or complemented with additional units or communication connections.

### Aspect 330SE eHealth General Communication Housing

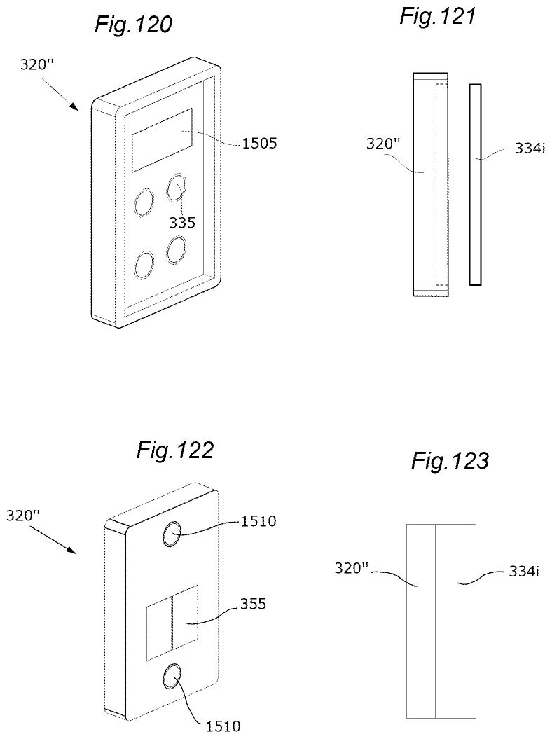

As have been discussed before in this application, communication with a medical implant needs to be reliable and secure. For this purpose, it is desirable to have a standalone device as an external remote control (for example described as 320″ in FIGS. 119a-119h) for the medical implant, such that no other programs or applications run on the same device which may disturb or corrupt the communication to the medical implant. However, the smartphone or tablet (for example described as 334 in FIGS. 119a-119h) has become an integrated part of everyday life for most people. This means that we almost always have our smartphones at hand. For this reason, it would have been convenient for the patient to communicate with the medical implant directly using the smartphone, such that no additional standalone device would have to be carried. However, as a lot of other applications are running on the smartphone, it does not fulfill the requirement of being a secure and reliable communication tool without interference from other communication. It is therefore desirable to split the tasks of providing secure communication between the external device and the implant from the task of communicating with the Internet and providing a familiar and intuitive user interface. For this purpose, and external device providing secure communication and tamperproof soft- and hardware, where the display device allows for intuitive and easy use is provided. In the embodiments described with reference to FIGS. 120-124 a device fulfilling these combinatory needs will be described in the form of a standalone remote control external device integrated in a housing unit 320″ connectable to a smartphone or another display device 334, such as a smart watch or a tablet.

FIG. 120 shows the housing unit 320″ in an elevated perspective view form the left, and FIG. 121 shows the housing unit 320″ in a plain view from the left. In the embodiment shown in FIG. 120, the housing unit 320″ has a rectangular shape with rounded edges, having a height 1521 which is more than 1.5 times the width 1522. The housing unit 320″ comprises recess 1525 configured to receive a display device 334, in the form of a smartphone, configured to be fitted in the housing unit 320″ for mechanically, disconnectably connecting the display device 334 to the housing unit 320″. The boundaries of the recess 1525 in the housing unit 320″ forms an edge 1528 configured to encircle the display device 334, when the display device 334 is inserted into the recess 1525. In the embodiment shown in FIG. 120, the recess 1525 has a depth 1526 configured to allow the display device 334 to be entirely inserted into the recess 1525. As such, the depth 1526 of the recess 1525 exceeds the depth 1531 of the display device 334. In the embodiment shown in FIGS. 120 and 121, the edge is relatively thin, and has a width 1527 which is in the range ⅛- 1/100 of the width of the display device 334, as such, the housing unit 320″ has a width in the range 1.02-1.25 times the width 1522 of the housing unit 320″. In the same way, the housing unit 320″ has a height 1521 in the range 1.01-1.25 times the height 1521 of the display device 334. In the embodiment shown in FIGS. 120-121, the edges 1528 are configured to clasp the display device 334 and thereby mechanically fixate the display device 334 in the housing unit 320″. The minimum bounding box of the housing unit 320″ and the display device 334 when mechanically connected, is no more than, 10% wider, 10% longer or 100% higher, than the minimum bounding box of the display device 334.

For creating a clasping fixation, the edges of the housing unit 320″ is made from an elastic material crating a tension between the edge 1528 and the display device 334 holding the display device 334 in place. The elastic material could be an elastic polymer material, or a thin sheet of elastic metal.

For the purpose of further fixating the display device 334 in the housing unit 320″, the inner surface of the edges 1528 may optionally comprise a recess or protrusion (not shown) corresponding to a recess or protrusion of the outer surface of the display device 334. The edges 1528 may in the alterative comprise concave portions for creating a snap-lock clasping mechanical fixation between the housing unit 320″ and the display device 334.

In the embodiment shown in FIGS. 120 and 121, the housing unit 320″ functions as a remote control for communicating with an implanted medical device, including receiving information from, and providing instructions and updates to, the implanted medical device. Information could be information related to a state of the implanted medical device including any functional parameter of the implanted medical device or could be related to a state of the patient, including any physiological parameter pertaining to the body of the patient (further described on other sections of this disclosure). For the purpose of providing input to the implanted medical device and controlling and updating the functions of the housing unit 320″, the housing unit 320″ comprises a control interface comprising switches in the form of control buttons 335. The control buttons 335 are configured to be used when the external device is disconnected from the display device 334. The control interface further comprises a display 1505, which is a smaller and typically less sophisticated display 1505 than the display of the display device 334. In an alternative embodiment, the control buttons 335 and display 1505 are integrated into a single touch-responsive (touchscreen) display on which the control buttons may be displayed. In the embodiment shown in FIGS. 120 and 121, one of the control buttons 335 is a control button for activating the implanted medical device and another of the control buttons 335 is a control button for deactivating the implanted medical device. When the display device 334 is attached to the housing unit 320″, the control buttons 335 and the display is covered by the display device 334 and are as such not in an operational state. In the embodiment shown in FIGS. 120 and 121, the housing unit 320″ is configured to transmit information pertaining to the display of the user interface to the display device 334 and the display device 334 is configured to receive input pertaining to communication to or from the implantable medical device from the patient, and transmit signals based on the received input to the housing unit 320″. The input may be a command to change the operational state of the implantable medical device. The display device 334 comprises a touch screen configured to display the user interface and receive the input from the patient. The display of the display device 334 may comprise one or more OLEDs or IPS LCDs elements. When the display device 334 is connected to the housing unit 320″, the display device 334 is configured to display a control interface which is used to communicate with the housing unit 320″, i.e. providing input to and receiving information from the housing unit 320″. The input provided the housing unit 320″ is then relayed to the implanted medical device—and in the same way information communicated from the implanted medical device to the housing unit 320″ may be relayed or displayed on the display device 334. Having an external device comprising a combination of a housing unit 320″ comprising the communication means for communicating with the implanted medical device and a display device 334 basically only functioning as and Input/Output device connected to the housing unit 320″ makes it possible to have a secure communication between the housing unit 320″ and the display device 334, which is out of reach from the Internet connection of the display device 334, which makes it much harder for an external attacker to get access to any of the vital communication portions of the housing unit 320″. The communication between the housing unit and the display device 334 is very restricted and the only communication allowed from the display device 334 to the housing unit 320″ is input from the patient or a healthcare professional, and authentication parameters created by an authentication application running on the display device 334. The authentication application running on the display device 334 could be a number-generating authenticator or a biometric authenticator for authenticating the patient or health care professional, and the authentication parameters could for example be parameters derived from a facial image or a fingerprint. In the opposite direction, i.e. from the housing unit 320″ to the display device 334, the communication could be restricted to only communication needed for displaying information and/or a graphical user interface on the display device 334. The communication restrictions could for example be based on size of the communication packages or the frequency with which the communication takes place which reduces the risk that an un-authorized person makes multiple attempts to extract information from, or transit information to, the hand-held device.

In the embodiment shown with reference to FIGS. 120 and 121, the housing unit 320″ comprises a first communication unit providing a wireless connection 413 to the display device 334. The wireless connection 413 is in the embodiment shown in FIGS. 120 and 121 based on NFC, but could in alternative embodiment be based on Bluetooth or any other communication pathway disclosed herein. The housing unit 320″ further comprises a second communication unit providing a wireless connection with the implanted medical device. The wireless communication between the housing unit 320″ and the implanted medical device is in the embodiment shown in FIGS. 120 and 121 based on Bluetooth, but could in alternative embodiments be based on NFC or UWB or any other communication pathway disclosed herein.

As mentioned, in the embodiment shown in FIGS. 120 and 121, the wireless communication between the housing unit 320″ and the display device 334 is based on NFC, while the wireless communication between the housing unit 320″ and the is based on Bluetooth. As such, the first communication unit of the housing unit 320″ is configured to communicate wirelessly with the display device 334′ using a first communication frequency and the second communication unit of the housing unit 320″ is configured to communicate wirelessly with the implantable medical device using a second different communication frequency. For this purpose, the first communication unit of the housing unit 320″ comprises a first antenna configured for NFC-based wireless communication with the display device 334, and the second communication unit comprises a second antenna configured for Bluetooth-based wireless communication with the implantable medical device. The first and second antennae may be a wire-based antennae or a substrate-based antennae. As such, the first communication unit is configured to communicate wirelessly with the display device 334 on a first frequency and the second communication unit is configured to communicate wirelessly with the implantable medical device using a second different communication frequency. Also, first communication unit of the housing unit 320′ is configured to communicate wirelessly with the display device 334 using a first communication protocol (the NFC-communication protocol), and the second communication unit is configured to communicate wirelessly with the implantable medical device using a second communication protocol (the Bluetooth communication protocol). The first and second communication protocols are different which adds an additional layer of security as security structures could be built into the electronics and/or software enabling the transfer from a first to a second communication protocol.

In an alternative embodiment, the second communication unit may be configured to communicate wirelessly with the implantable medical device using electromagnetic waves at a frequency below 100 kHz, or preferably at a frequency below 40 kHz. The second communication unit may thus be configured to communicate with the implantable medical device using “Very Low Frequency” communication (VLF). VLF signals have the ability to penetrate a titanium housing of the implant, such that the electronics of the implantable medical device can be completely encapsulated in a titanium housing. In yet further embodiments, the first and second communication units may be configured to communicate by means of an RFID type protocol, a WLAN type protocol, a BLE type protocol, a 3G/4G/5G type protocol, or a GSM type protocol.

In yet other alternative embodiments, it is conceivable that the mechanical connection between the housing unit 320″ and the display device 334 comprises an electrical connection for creating a wire-based communication channel between the housing unit 320″ and the display device 334. The electrical connection could also be configured to transfer electric energy from the display device 334 to the housing unit, such that the housing unit 320″ may be powered or charged by the display device 334. A wired connection is even harder to access for a non-authorized entity than an NFC-based wireless connection, which further increases the security of the communication between the housing unit 320″ and the display device 334.

In the embodiment shown with reference to FIGS. 120 and 121, the display device 334 comprises a first communication unit providing a wireless connection 413 to the housing unit 320″ based on NFC. The display device 334 further comprises a second communication unit providing a wireless connection with a further external device and/or with the Internet. The second external device may be far away, for example at a hospital or a place where a medical professional practice. The wireless communication between the display device 334 and a further external device is in the embodiment shown in FIGS. 120 and 121 based on Wi-Fi, but could in alternative embodiments be based on for example Bluetooth.

As mentioned, in the embodiment shown in FIGS. 120 and 121, the wireless communication between the display device 334 and the housing unit 320″ is based on NFC, while the wireless communication between the display device and a further external unit is based on Wi-Fi. As such, the first communication unit of the display device 334 is configured to communicate wirelessly with the housing unit 320″ using a first communication frequency and the second communication unit of the display device 334 is configured to communicate wirelessly with a further external device using a second different communication frequency. For this purpose, the first communication unit of the display device 334 comprises a first antenna configured for NFC-based wireless communication with the housing unit 320″, and the second communication unit comprises a second antenna configured for Wi-Fi-based wireless communication with a further external device. The first and second antennae may be wire-based antennae or substrate-based antennae. As such, the first communication unit is configured to communicate wirelessly with the housing unit 320″ on a first frequency and the second communication unit is configured to communicate wirelessly with the further external device using a second different communication frequency. Also, the first communication unit of the display device 334 is configured to communicate wirelessly with the housing unit 320″ using a first communication protocol (the NFC communication protocol), and the second communication unit is configured to communicate wirelessly with the further external device using a second communication protocol (the Wi-Fi communication protocol). The first and second communication protocols are different which adds an additional layer of security as security structures could be built into the electronics and/or software enabling the transfer from a first to a second communication protocol.

In alternative embodiments, the second communication unit of the display device 334 may be configured to communicate with the further external device by means of, a WLAN type protocol, or a 3G/4G/5G type protocol, or a GSM type protocol.

In the embodiment shown in FIGS. 120 and 121, the communication range of the first communication unit of the housing unit 320″ is less than a communication range of the second communication unit of the housing unit 320′, such that the communication distance between the housing unit 320″ and the medical implant may be longer than the communication distance between the housing unit 320″ and the display device 334. In the embodiment shown in FIGS. 120 and 121, the communication range of the first communication unit may be constrained to a length that is less than five times the longest dimension of the minimal bounding box of the display device 334, or more precisely constrained to a length that is less than three times the longest dimension of the minimal bounding box of the display device 334.

In the embodiment shown in FIGS. 120 and 121, communication between the housing unit 320″ and the display device 334 is only enabled when the housing unit 320″ is connected to the display device 334. I.e. at least one of the housing unit 320″ and the display device 334 is configured to allow communication between the housing unit 320″ and the display device 334 on the basis of the distance between the housing unit 320″ and the display device 334. In the alternative, the housing unit 320″ and/or the display device 334 may comprise a sensor configured to estimate whether the housing unit 320″ is attached to the display device 334 or not, such as a mechanically activated switch or a photo resistive sensor which providing sensor input when the housing unit 320″ and display device 334 are mechanically connected to each other. The signal from the at least one sensor then may be used to permit usage of the communication unit configured for communication with the display device 334.

In the embodiment shown in FIGS. 120 and 121, communication between the housing unit 320″ and the implantable medical device is only enabled on the basis of a distance between the housing unit 320″ and the implantable medical device. In the embodiment shown in FIGS. 120 and 121, the distance should be less than twenty times the longest dimension of the minimal bounding box of the display device, or more specifically less than ten times the longest dimension of the minimal bounding box of the display device. The distance between the housing unit 320″ and the medical implant may be measured using electromagnetic waves, or acoustic waves. The process of measuring the distance may comprise triangulation.

In the embodiment shown in FIGS. 120 and 121, the second communication unit of the display device 334 need to be disabled to enable communication between the display device 334 and the housing unit 320″, and further the second communication unit of the display device 334 needs to be disabled to enable communication between the housing unit 320″ and the medical implant. Also, the second communication unit of the housing unit 320″ needs to be disabled to enable communication between the housing unit 320″ and the medical implant.

In the embodiment shown in FIGS. 120 and 121, the housing unit 320″ further comprises an encryption unit configured to encrypt communication received from the display device 334 before transmitting the communication to the implanted medical device. The encryption unit may for example be based on one of the following algorithms: AES, Blowfish, DES, Kalyna, Serpent or Twofish. For the purpose for handling the communication, I/O and encryption, the housing unit 320″ comprises a processor which could be a general-purpose microprocessor and/or an instruction set processor and/or related chips sets and/or special purpose microprocessors such as ASICs (Application Specific Integrated Circuit). The processor also comprise memory for storing instruction and/or data.

The housing unit 320″ may comprise a storage unit, such as a battery, for storing energy. The storage unit may be adapted to be charged by the display device 334, or another external device. In some examples, the charging is performed using reverse wireless charging. To that end, the housing unit 320″ may comprise an energy receiver connected to the storage unit, the energy receiver being adapted to wirelessly receive energy from another device. The display device 334 may comprise a primary coil and the housing unit comprise a secondary coil connected to an energy storage of the housing unit, wherein the display device 334 is adapted to wirelessly charge the housing unit using the first coil, and wherein the housing unit is adapted to receive wirelessly transmitted energy through the second coil and store the energy in the storage unit. In one example, the wireless charging may be performed using the Qi standard for wireless charging.

FIGS. 122 and 123 shows an embodiment of the external unit similar to the embodiment described with reference to FIGS. 120 and 121. The difference being that in the embodiment of FIGS. 122 and 123, the housing unit 320″ does not clasp the display device 334. Instead, the housing unit comprises two magnets 1510 for magnetically fixating the display device 334 to the housing unit 320″.

In alternative embodiments, it is equally conceivable that the external device comprises an intermediate portion, which is fixedly fixated to the housing unit for providing a detachable connection with the display device 334. In the alternative, the intermediate device could be fixedly fixated to the display device 334 and provide a detachable connection with the housing unit 320″.

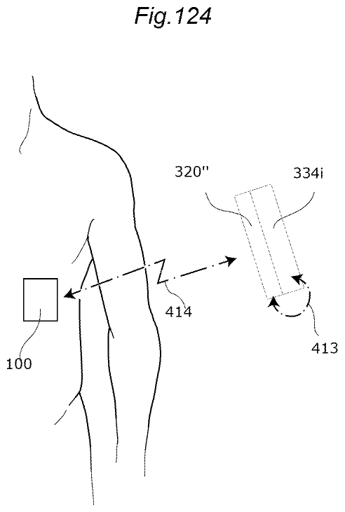

FIG. 124 shows a system overview of the external device (which could be the external device of the embodiment described with reference to FIGS. 120 and 121, or of the embodiment described with reference to FIGS. 122 and 123). The housing unit 320″ is connected to the display device 334. A wireless connection 413 is provided between the housing unit 320″ and the display device 334, and a further wireless connection 413 is provided between the housing unit 320″ and the implanted medical device 100, such that the housing unit can send instructions and updates to the implanted medical device 100, and receive information, parameters (such as sensor values) and alarms from the implanted medical device 100. The communication between the external device and the medical implant 100 is further described in other portions of this disclosure.

In some examples shown in any of FIGS. 122-124, the patient remote control or the patient EID may comprise a wireless transceiver for communication with the implant, and a second wireless receiver for communication with a communications network over which the patient remote control or patient EID may communicate with a patient remote control, patient EID, HCP EID or DDI.

In some examples, the patient remote control or the patient EID may be wirelessly charged. Thus, the patient remote control or the patient EID may comprise a first coil for receiving wireless energy to be used or stored at the patient remote control or the patient EID.

The implantable medical device 100 may be an active and/or operable implantable medical device 100 which may be an implantable medical device configured to exert a force on a body portion of the patient. The body portion of the patient may be a fluid carrying vessel, an organ, a joint, a membrane, a muscle, a bone or a nerve. The implantable medical device 100 may comprises an electrical motor and a controller for controlling the electrical motor and instructions transmitted to the implantable medical device 100 could be instructions pertaining to the control of the electrical motor. The controller may control, the velocity, the acceleration or the torque of the motor. The implantable medical device 100 could for example comprises at least one of an external heart compression device, an apparatus assisting the pump function of a heart of the patient, an apparatus assisting the pump function comprising a turbine bump placed within a patient's blood vessel for assisting the pump function of the heart, an operable artificial heart valve, an operable artificial heart valve for increasing the blood flow to the coronary arteries an implantable drug delivery device, an implantable drug delivery device for injecting directly into a blood vessel and change the position of the injection site, all from within the patient's body, an implantable drug delivery device for injecting potency enhancing drugs into an erectile tissue of the patient, a hydraulic, mechanic, and/or electric constriction implant, an operable volume filling device, an operable gastric band, an operable implant for stretching the stomach wall of the patient for creating satiety, an implant configured to sense the frequency of the patient ingesting food, an operable cosmetic implant, an operable cosmetic implant for adjust the shape and/or size in the breast region of a patient, an implant controlling medical device for the emptying of a urinary bladder, an implant hindering urinary leakage, an implant hindering anal incontinence, an implant controlling the emptying of fecal matter, an implant monitoring an aneurysm, an implant for hindering the expansion of an aneurysm, an implant lubricating a joint, an implant for affecting the blood flow to an erectile tissue of the patient, an implant for simulating the engorgement of an erectile tissue, an implant with a reservoir for holding bodily fluids, an implant storing and/or emptying a bodily reservoir or a surgically created reservoir, an implant communicating with a database outside the body, an implant able to be programmed from outside the body, an implant able to be programmed from outside the body with a wireless signal, an implant treating impotence, an implant controlling the flow of eggs in the uterine tube, an implant controlling the flow of sperms in the uterine tube, an implant controlling the flow of sperms in the vas deferens, an implant for hindering the transportation of the sperm in the vas deferens, an implant treating osteoarthritis, an implant performing a test of parameters inside the body, an implant controlling specific treatment parameters from inside the body, an implant controlling bodily parameters from inside the body, an implant controlling the blood pressure, an implant controlling the blood pressure by affecting the dilatation of the renal artery, an implant controlling a drug treatment parameter, an implant controlling a parameter in the blood, an implant for adjusting or replacing any bone part of a body of the patient, an implant replacing an organ of the patient or part of an organ of the patient or the function thereof, a vascular treatment device, an implant adapted to move fluid inside the body of the patient, an implant configured to sense a parameter related to the patient swallowing, an implant configured to exercise a muscle with electrical or mechanical stimulation, an implant configured for emptying an intestine portion on command, an operable implant configured to be invaginated in the stomach of the patient to reduce the volume of the stomach substantially more than the volume of the device, an implant configured for emptying the urinary bladder from within the patient's body by compressing the bladder, an implant configured for draining fluid from within the patient's body, an implant configured for the active lubrication of a joint with an added lubrication fluid, an implant configured for removing clots and particles from the patient's blood stream, an implant configured for elongating or straightening a bone in the patient, to reduce scoliosis, a device to stimulate the brain for a several position to a focused point, an artificial stomach replacing the function of the natural stomach, an implant configured for adjusting the position of a female's urinary tract or bladder neck, an implant configured for stimulating the ampulla vas deference and creating temporary constriction.

### Aspect 447A, B, C Data_Packet _Encryption-Implant, External, Method

An implantable medical device 100 configured to receive remote instructions from an external system will now be described with reference to FIGS. 119a-h. The implantable medical device 100 comprises a wireless receiver configured to receive wirelessly transmitted data packets from an external device 320′, 320″, 320′″ and a computing unit configured to verify the electronic signature, and use a checksum provided in the data packet to verify the integrity of the instructions. The wireless receiver mat be part of a wireless transceiver. The computing unit may comprise a memory unit configured to store electronic signatures, and wherein the computing unit is configured to verify the electronic signature my comparing the electronic signature with the electronic signatures stored in the memory unit.

The computing unit may be configured to decrypt the data packet. The computing unit may be configured to use the checksum to verify that the bit stream making up the instructions is unchanged.

As an alternative, the date packet may be decrypted using the methods and/or systems described with reference to aspect 331.

In one example, the implantable medical device 100 comprises a control program configured to control at least one function of the implantable medical device. The computing unit may thus be configured to alter the control program on the basis of the received instructions.

The implantable medical device 100 may comprise an internal computing unit configured to run a control program for controlling a function of the implantable medical device 100, wherein the control program comprises at least one adjustable parameter affecting the control of the implantable medical device, and wherein the method of providing remote instructions comprises providing instructions for altering the at least one parameter for affecting the control of the implantable medical device.

The computing unit may comprise a memory unit configured to store parameter values. The implantable medical device may be configured to verify that the instructions for altering the at least one parameter will result in the at least one parameter being updated to a parameter value comprised in the set of stored parameter values. In some examples, the implantable medical device is configured to at least one of decrypting the data packet and verifying the electronic signature using a private key of the implantable medical device.

The private key may be provided in the implantable medical device by the manufacturer of the implantable medical device. In some examples, the private key is stored as hardware or software in the implantable medical device. The private key may be a non-extractable key. In some examples the private key is a physical unclonable function.

To further increase the security, the wireless receiver or wireless transceiver may be configured to be placed in an off-mode, in which no wireless communication can be received by the wireless transceiver, and wherein the set of rules comprises a rule stipulating that communication from the central unit is only accepted at the security module when the wireless transceiver is placed in the off-mode.

According to an embodiment, the implantable medical device is configured to perform a proof of possession operation comprising transmitting, from the implantable medical device to the external system, a query based on a public key associated with the private key of the external system, receiving, at the implantable medical device, a response based on the possession of the private key in the external system, and verifying that the response based on the possession of the private key matches the query based on a public key.

The implantable medical device may be configured to communicate with the external system independently of time, for example by using single use codes, as described in aspect 447D.

The implantable medical device may be configured to verify a first electronic signature made using at least one of a first key and a second key, and verifying a second electronic signature made using at least one of a first key and a second key. At least one of the first and second keys mat be a private key, and/or the first and second keys are different. In some examples, the first and second keys comprises at least one common element.

T the implantable medical device may be configured to verify a first electronic signature to allow communication from the external system to the implantable medical device, and verify a second electronic signature to allow an instruction received in the communication to alter the control program running on the implantable medical device. The first electronic signature may be an electronic signature linked to the user of the implantable medical device and the second electronic signature may be an electronic signature linked to a healthcare provider.

In some examples, only a portion of the private key is needed to at least one of: decrypt the data packet and verify the electronic signature.

In some examples, the implantable medical device trusts any external device holding the private key.

The implantable medical device may be configured to receive the data packet comprising at least one instruction signed by a private key of the external system, and a public key including information about which root have created the public key. In some examples, the implantable medical device is configured to accept communication from an external system based on at least one password being provided to the implantable medical device. The implantable medical device may be configured to accept communication from an external system based on two passwords being provided to the implantable medical device. For examples, the implantable medical device is configured to accept communication from an external system based on one patient password and one healthcare provider passwords being provided to the implantable medical device.

A corresponding external device 320′, 320″, 320′″ will now be described with reference to FIGS. 119a-h. An external system or external device for providing remote instructions to an implantable medical device is shown. The external system, or any of the external devices therein 320′, 320″, 320′″, is configured to instructions to be transmitted to the implantable medical device, derive a checksum from the instructions, electronically sign the instructions and the checksum, form a data packet from the instructions, the electronic signature and the checksum, wherein the external system comprises a wireless transmitter configured to wirelessly send the data packet to the implantable medical device.

The external system, may comprise comprises a first external device 320′ and a second external device 320″, and wherein the first external device 320′ is configured to transmit the data packet to the second external device 320″, and wherein the second external device 320″ is configured to transmit the data packet wirelessly to the implantable medical device 100 without changing the data packet.

Alternatively, the first external device 320′ is configured to transmit the data packet to the second external device 320″, and wherein the second external device 320″ is configured to transmit the data packet wirelessly to the implantable medical device 100 without full decryption of the data packet.

The first external device 320′ may be configured to send the data packet from the first external device 320′ to the second external device 320″ using a first network protocol the second external device 320″ may send the data packet to the implantable medical device 100 using a second network protocol.

In some examples, the first external device 320′ is configured to send the data packet from the first external device 320′ to the second external device 320″ using wired communication and send the data packet from the second external device 320″ to the implantable medical device 100 using wireless communication. If using wireless communication, the first external device 320′ may be configured to wirelessly send the data packet from the first external device 320′ to the second external device 320″ using a first frequency band, and wirelessly send the data packet from the second external device 320″ to the implantable medical device 100 using a second frequency band. In some examples, the first external device is configured to wirelessly send the data packet from the first external device to the second external device using a first wireless technology, and wirelessly send the data packet from the second external device to the implantable medical device using a second wireless technology.

The external system may be configured to transmit at least one instruction for altering the control program of the implantable medical device 100, to the implantable medical device 100, and/or the external system may be configured to provide at least one instruction to the implantable medical device 100 for altering at least one parameter for affecting the control of the implantable medical device 100. In some examples, the external system is configured to provide at least one instruction for updating at least one parameter of the control program to a parameter value comprised in a set of parameter values stored in the implantable medical device 100.

The external system may be configured to electronically sign the instructions at the external system using a key of the external system. Details of the key, and any use of a first and a second key, have been discussed above with reference to the implantable medical device 100.

The first external device may be configured to form the data packet and electronically sign the instruction using a first private key, and the second external device is configured to: receive the data packet from the first external device, verify that the first external device is a trusted transmitter, in response to the verification, electronically sign the data packet using a second private key, and transmit the data packet from the second external device to the medical implant. The checksum is configured to verify that no changes have been made to the bit stream forming the instructions.

The key may be placed on a key device external to the any of the external devices. In some examples, the key device comprises a wireless transmitter for wirelessly transmitting the at least one private key or a signal based on the private key, to the first external device. The key device may, for example, comprise at least one of: a key card, a wearable device and a handset.

The external device may be locker for an increased security. The external device may thus be unlocked by a user providing credentials to the external device. Such credentials may, for example, comprise a username, a password, a PIN-code, or any other credential.

An improved security may further be achieved by combining the embodiments described with reference to Aspects 447A-C with any of the methods disclosed in aspects 36, 307, 308, 399, 400, 401, and 402, which relate to methods for communicating instructions or updates to the implantable medical device using any of the external devices shown in FIGS. 119a-h. Aspects 447A-C may advantageously be combined with the use of single use codes, as will be described below with reference to aspect 447D.

### Aspect 447D Single-Use_Codes_Encryption

A method of providing remote instructions from an external system to an implantable medical device, wherein the implantable medical device comprises a list of codes and the external system comprises a list of codes, will now be described with reference to FIGS. 119a-h. The method may be performed by the system(s) shown in FIGS. 119a-h. The method comprises encrypting the instructions at the external system or at an external device 320′, 320″, 320′″ using a code from a position on the list of codes, wirelessly sending the encrypted instructions to the implantable medical device 100, and decrypting, at the implantable medical device 100, the instructions using a code from a position on the list of codes.

The method may further comprise the steps of wirelessly sending position information from the external device 320′, 320″, 320′″ to the implantable medical device 100, and using the information at the implantable medical device 100 for selecting the code from the list of codes.

The step of encrypting, at the external system or at the external device 320′, 320″, 320′″, the instructions using a code from a position on the list of codes comprises selecting the code on a current position on the list of codes, wherein the method further comprises the step of updating the current position to a new current position after using the code.

The step of decrypting, at the implantable medical device 100, the instructions using a code from a position on the list of codes comprises selecting the code on a current position on the list of codes, wherein the method further comprises the step of updating the current position to a new current position after using the code.

The current position may comprise a number and wherein the step of updating the current position comprises updating the number to a sequential number.

The step of wirelessly sending the encrypted instructions to the implantable medical device 100 comprises sending the encrypted instructions from a first external device 320′ to a second external device 320″ and further wirelessly sending the encrypted instructions from the second external device 320″ to the implantable medical device 100. The second external device may transmit the encrypted instructions without changing the encrypted instructions, and/or the encrypted instructions without full decryption.

The implantable medical device 100 may comprise a control program configured to control at least one function of the implantable medical device 100, and wherein the method further comprises altering the control program on the basis of the received instructions. The implantable medical device 100 may comprises internal computing unit configured to run a control program for controlling a function of the implantable medical device 100, wherein the control program comprises at least one adjustable parameter affecting the control of the implantable medical device 100, and wherein the method of providing remote instructions comprises providing instructions for altering the at least one parameter for affecting the control of the implantable medical device 100.

The implantable medical device 100 may comprise a set of stored parameter values, and wherein the method further comprises the step of verifying that the instructions for altering the at least one parameter will result in the at least one parameter being updated to a parameter value comprised in the set of stored parameter values.

In the same way as discussed above with reference to aspects 447A-C, the communication between the implantable medical device 100 and the first and/or second external device 320′, 320″, 320′″ may be performed using wired or wireless technology, different protocols and/or different frequency bands.

The method may be combined with the security module as described with reference to aspect 331.

The step of electronically signing the instruction at the external system, may be performed and using keys as discussed with reference to Aspects 447A-C.

### Aspect 454: Dual Remote Controls

FIG. 65L shows one embodiment of a communication system for transmission of data to and/or from an implantable medical device 100. The communication system comprises an implantable medical implant, a first remote control 320″, and a second remote control 320″″. The first remote control 320″ (also referred to as patient external device 320″) is operable by a user and comprises a first wireless communication unit configured for wireless transmission of data to and/or from the implantable medical device 100. The second remote control 320″″ is inoperable by a user (i.e. may not comprise any input means such as buttons, switches, or user interface to receive any input directly from a user) and comprises a second wireless communication unit configured for wireless transmission of control commands and/or data to and/or from the implantable medical device 100. The second remote control 320″″ further comprises a third communication unit for communicating with a patient display device 334 (e.g. a smartphone, smartwatch, tablet, and/or the like).

By having two separate remote controls, the security of the implant may be improved, as there are two separate ways of controlling the implant. Thus, in case of a malfunction of either of the remote controls, the implant may still be controlled. Furthermore, this allows for the second remote control to be smaller or more compact since it is inoperable by the user other than through a patient display device or another external device. The second remote control may thus be smaller and potentially less expensive.

The first and second remote controls 320″, 320″″each comprise a wireless transceiver 328 for communicating with the implantable medical device 100. The first and/or second remote control 320″, 320″″ is capable of controlling the operation of the implantable medical device 100 via the controller 300 (for controlling the implantable medical device and for communicating with devices external to the body of the patient and/or implantable sensors). The first and/or second remote control 320″, 320″″ may control the operation of the implantable medical device 100 by controlling pre-set functions of the implantable medical device 100, e.g. for operating an active portion of the implantable medical device 100 for performing the intended function of the implantable medical device 100.

The first and/or second remote control 320″, 320″″ is able to communicate with implantable medical device 100 using any standard or proprietary protocol designed for the purpose. At least one of the first remote control 320″, the second remote control 320″″, and the implantable medical device 100 may, e.g., comprise a Bluetooth (BT) transceiver. In particular, the wireless transceiver 328 may comprise a BT transceiver, and the and/or second remote control 320″, 320″″ may be configured to communicate with implantable medical device 100 using BT. In one embodiment, the first and/or second remote control 320″, 320″″ is configured to communicate with implantable medical device 100 using NFMI.

In an alternative configuration, the first and/or second remote control 320″, 320″″ may communicate with the implantable medical device 100 using a combination of Ultra-Wide Band (UWB) wireless communication, NFMI and/or BT. For example, at least one of first remote control 320″, the second remote control 320″″, and the implantable medical device 100 may comprise a UWB transceiver. The use of UWB technology enables positioning of the first and/or second remote control 320″, 320″″which can be used by the implantable medical device 100 as a way to establish that the first and/or second remote control 320″, 320″″ is at a position which the implantable medical device 100 and/or the patient can acknowledge as being correct, e.g. in the direct proximity to the medical device 100 and/or the patient, such as within reach of the patient and/or within 1 or 2 meters of the implantable medical device 100.

When a combination of BT and UWB and/or NFMI technology is used, the UWB or NFMI technology may be used for location-based authentication of the first and/or second remote control 320″, 320″″, whereas the communication and/or data transfer could take place using BT or any other way of communicating different from the UWB or NFMI. The UWB or NFMI signal could in some embodiments also be used as a wake-up signal for the controller 300, or for the BT transceiver, such that the BT transceiver in the implantable medical device 100 can be turned off when not in use, which eliminates the risk that the BT is intercepted, or that the controller 300 of the implantable medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The first remote control 320″ may be configured to control functions of the implantable medical device 100 based on user input to the first remote control 320″. In particular, the first remote control 320″ may comprise an input device for receiving a first user input, wherein the first remote control 320″ is configured to transmit the first user input to the implantable medical device 100. The first remote control 320″ may comprise a computing unit 326 which runs a software application for communicating with the implantable medical device 100. The computing unit 326 may receive the first user input directly from control buttons 335 arranged on the first remote control 320″. The computing unit 326 may be configured to encrypt control commands before transmission to the implantable medical device 100.

The computing unit 326 is further configured to transform the received first user input into control commands for wireless transmission to the implantable medical device 100.

The second remote control 320″″ may comprise a wireless transmitter 325 configured for transferring energy wirelessly. The energy may be in the form of a magnetic field or any other signal such as electromagnetic, radio, light, sound or any other type of signal to transfer energy wirelessly to a wireless receiver 395 of the implantable medical device 100. The wireless receiver 395 of the implantable medical device 100 is configured to receive the energy in the form of the magnetic field and transform the energy into electric energy for storage in an implantable energy storage unit 40 of the implantable medical device 100, and/or for consumption in an energy consuming part of the implantable medical device 100 (such as the operation device, controller 300 etc.). In other words, the implantable energy storage unit 40 may be adapted to be wirelessly charged. The first remote control may similarly comprise a wireless transmitter for transferring energy wirelessly to the implantable medical implant.

The implantable energy storage unit 40 may particularly be connected to the wireless receiver 395 for receiving wireless energy from the first and/or second remote control 320″, 320″″.

In the embodiment shown in FIG. 694A, the second remote control 320″″ is configured to communicate with a patient display device 334. Here, the patient display device 334 comprises the consumer electronics device. In particular, the second remote control 320″″ is configured to receive a second user input from the patient display device 334 and to transmit the second user input to the implantable medical implant. The second remote control 320″″ may receive the second user input from a control interface 334i displayed on the patient display device 334 operated by the patient.

The patient display device 334 may for example be a mobile phone, a tablet or a smart watch. The display device 334 may, for example, communicate with the second remote control 320″″ by means of BT, but any wireless or wired communication means may be used. The control interface 334i, e.g. in the form of a web-view portal, may be transmitted from the second remote control 320″″ to the patient display device 334 over BT. Control commands in the form of inputs from the patient to the control interface 334i are transmitted from the patient display device 334 to the second remote control 320″″, providing input to the second remote control 320″″equivalent to the input that may be provided using the control buttons 335 or other input means of the first remote control 320″. The control commands created in the patient display device 334 may be encrypted in the patient display device 334 and transmitted to the second remote control 320″″using BT or any other communication protocol.

The second remote control 320″″ may be implemented and/or integrated in an accessory to the patient display device 334. The second remote control 320″″ may, e.g., form part of a mobile phone case (i.e. smartphone case) for a mobile phone. Alternatively, the second remote control 320″″ may be integrated in a case for a personal computer, or a body worn camera, or any other suitable type of external device as described herein. The case may for example be connected to the patient display device 334 (e.g. mobile phone) using a wire from the case and connected to the patient display device (e.g. a charging port).

The second remote control 320″″ may not be connected to the DDI or the Internet, thereby increasing security. The second remote control 320″″ may have a private key, in particular the second remote control 320″″ may be activated by a private key 333′ of the patient for a certain time period.

This may activate the function of the patient display device 334 and the remote wed-view display portal supplied by the second remote control to the patient display device 334.

The patient's private key 333′ may be supplied in a patient private key device comprising a smartcard that may be inserted or provided close to the first remote control 320″ and/or close to the second remote control 320″″ to activate a permission to communicate with the implantable medical device 100 for a certain time period. The patient's private key 333′ is in the embodiment shown in FIG. 694A in the form of a key card having an interface for communicating with the first remote control 320″, the second remote control 320″″, and/or another device or control. The NFC-transmitter 339 and/or the printed QR-code 344 may be used as means for accessing the control interface 334i of the display device 334. In addition, the display device 334 may require a pin-code and/or a biometric input, such as face recognition or fingerprint recognition, for controlling the implantable medical implant.

The patient display device 334 may comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the second remote control 320″″. Disconnecting the auxiliary radio connections reduces the risk that the integrity of the control interface 334i displayed on the patient display device 334 is compromised, or that the control interface 334i displayed on the patient's display device 334 is remote controlled by an unauthorized device.

The data transmitted in the communication system may comprises a control command for the medical implant. Hence, real-time, remote management of patient care is provided and settings of the medical implant may be adjusted, e.g., based on the patient's current health status. Thus, invasive procedures may be averted while efficiency of healthcare delivery and patient comfort may be improved. Furthermore, more responsive and/or personalized healthcare may be provided, as adjustments can be made promptly in response to changes in the patient's condition.

At least one of the first wireless communication unit of the first remote control 320″ and the second wireless communication unit of the second remote control 320″″ may be configured to send and/or receive data using near-field magnetic induction (NFMI). Thus, enhanced security and reliability of the communication system may be provided. NFMI creates a private, secure communication link that is difficult to intercept or disrupt due to the magnetic field being spatially confined and thus less susceptible to interference compared to traditional radio frequency communication. Furthermore, NFMI penetrate materials such as water and body tissue, making it particularly suitable for communication with medical implants.

Further, at least one of the first wireless communication unit and the second wireless communication unit may comprise a transmitter coil for modulating a magnetic field for transmitting the data. In turn, the implantable medical implant may comprise a receiving coil and an NFMI receiver connected to the receiving coil to receive the data. The transmitter coil(s), in conjunction with the receiving coil and NFMI receiver of the implantable medical implant, may provide efficient and reliable data transfer. The use of a magnetic field for data transmission, which is typically more energy-efficient than traditional radio frequency communication, may additionally reduce power consumption and thereby extend an operational period of the implantable medical implant.

The transmitter coil(s) may be configured to modulate a magnetic field, and the NFMI receiver may be adapted to measure the magnetic field in the receiving coil. A modulated magnetic field may enable the construction of specific signal patterns for the data transmission such that transmission of complex data sets is enabled.

At least one of the first wireless communication unit and the second wireless communication unit may further be configured to wirelessly charge the implantable medical implant using NFMI. In particular, at least one of the first wireless communication unit and the second wireless communication unit may be, and/or act as, the wireless transmitter 325 configured for transferring energy wirelessly

Similarly, the implantable medical implant may comprise a coil for receiving wireless energy for charging the implant via NFMI. The coil of the implantable medical implant may, e.g., form part of, or be, the wireless receiver 395.

The second and third communication units of the second control unit 320″″ may be configured to transmit and/or receive data using different network protocols. In other words, the second and third communication units may be designed to send and/or receive data using separate and/or alternate networking standards. Thus, the communication system can communicate across a variety of network environments and conditions. A multi-protocol support may enhance interoperability of the second remote control 320″″, allowing for communicate with a wide range of devices and systems (such as the patient display device 334 and the implantable medical device 100). Alternatively, or additionally, the second and third communication units may for the same reasons be configured to transmit and/or receive data using different frequency bands.

The standard, communication, and/or network protocols discussed herein may be any one or more from the list of: Radio Frequency type protocol, RFID type protocol, WLAN type protocol, Bluetooth type protocol, BLE type protocol, NFC type protocol, 3G/4G/5G type protocol, and GSM type protocol.

In an example, the second communication unit has a longer effective range than the third communication unit. In other words, the second communication unit may be able to communicate with a device (e.g., the implantable medical device 100) from a further distance than the distance at which the third communication unit is able to communicate with another device (e.g., the patient display device 334). For example, the second communication unit may use a network protocol with a longer effective range than the network protocol of the third communication unit.

In the specific embodiment disclosed in FIG. 694A, the wireless connections between the different units are as follows. The wireless connection 413 between the patient display device 334 and the second remote control 320″″ is based on BT or any other communication protocol disclosed herein. The wireless connection 414 between the second remote control 320″″ and the implantable medical device 100 is based on BT and UWB or any other communication protocol disclosed herein. The wireless connection 416 between the first remote control 320″ and the implantable medical device 100 is based on BT, UWB, and the charging signal, or any other communication or energizing pathway disclosed herein.

The wireless connections specifically described in the embodiment shown in FIG. 694A may be replaced or assisted by wireless connections based on radio frequency identification (RFID), near field communication (NFC), Bluetooth, Bluetooth low energy (BLE), or wireless local area network (WLAN).

The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

An embodiment in which the second remote control 320″″ is comprised in a housing is shown in FIG. 694B.

### Aspect 453: Voice Control

FIG. 695A schematically shows a medical implant 10 when implanted in a patient 1. The medical implant comprises a processor 1300 connected to a medical device 100, which may be comprised in the implant 100. The medical device may be any medical device or implant discussed herein, and may be configured to control or monitor a function of the body of a patient. The medical implant 10 may further comprise or be connected to a microphone 1369. The microphone 1369 may receive audio and transfer that audio to the processor 1300. The processor 1300 may thus receive audio via microphone 369.

The processor 1300 may have two modes of operation, a learning mode for learning voice commands and an operational mode for recognizing and transmitting voice commands to the medical device 110 or the medical implant 1100. The processor 1300 may be configured to, when in the learning mode, receive a first audio training phrase and creating a transfer function, the transfer function being based on the first audio training phrase, wherein the transfer function is configured to adjust the amplitude of at least one frequency of audio received at the medical device 110 for enhancing audio received at the medical implant 100 to facilitate detection of voice commands. To this end, the processor 1300 may comprise a transfer function unit 1370. The processor 1300 may be further adapted to receive a second audio training phrase, the second audio training phrase comprising a voice command, wherein the voice command comprises an instruction for the control of the medical implant 1100 and/or the medical device 110. The processor 300 may be further configured to use the transfer function for generating an enhanced second audio training phrase in the medical implant, and associating the enhanced second audio training phrase with the instruction for the control of the medical implant. Thus, the medical implant 110 has learned that the voice command comprised in the enhanced audio training phrase corresponds to the instruction.

In some examples, the audio training phrases are inputted into a transfer function unit 1370 for creating the transfer function. The processor may further comprise a learning unit 1371 for associating the enhanced second audio training phrase with the instruction for the control of the medical implant.

The learning unit 1371 may, for example, comprise an algorithm based on machine learning for learning to associate the enhanced audio training phrase with the correct instruction for the medical device 1100. The voice commands, the instructions and any association between the voice commands may be stored in a memory unit 1373 comprised in or connected to the processor 1300.

The processor 1300 may be further adapted to receive audio input, process the audio input in order to determine an instruction and to transfer that instruction to the medical device 1100. In order to determine the instruction, the processor may use the transfer function 1370 to enhance the audio input and then determine the instruction associated with the enhanced audio input (as associated by the method described herein). The instruction may also be called a control command or a command. The instruction may be determined by and/or be transferred to the medical device 1100 via a command unit 1372 comprised in or connected to the processor 1300. The instruction may relate to a function of the medical device 1100 and may cause the medical device 1100 to perform an action, or it may relate to any other function of the medical implant 10, such as the processor 1300.

By learning voice commands, it may be meant that the processor associates an audio input with a control command for the medical device.

The processor 1300 may be further configured to, when in the operational mode, receive an audio command phrase for the medical device 1100 or implant 110. The processor 1300 may be further configured to apply a transfer function to create an enhanced audio command phrase. The transfer function may have been created as discussed above. The processor 1300 may determine a corresponding command for the medical based on the enhance audio command phrase, and send the command to the medical device 1100 or the medical implant 110. The medical device 1100 or implant 110 may then execute the command.

When the medical implant is implanted in the body, typically the medical implant stays in the same place in the body. Thus, it has been realized that any noise or distortion created by the body to audio commands may be substantially the same. By creating a transfer function based on a first audio training phase when the medical implant is implanted in the body, any noise created by the body or any distortions to the audio training phase caused by the body itself can be accounted for. The method thus accounts for that that the noise and distortions created by the body is substantially the same over time. Thus, the transfer function may account for those disturbances when enhancing any audio received by the medical implant. In this way, audio received by the medical implant may be enhanced, i.e., any known disturbances created by the body to the audio may be accounted for, before the medical implant does any further processing. Since the audio is enhanced before any training or processing, the process of recognizing which command for the medical implant the audio relates to may be simplified. That is, the processing power needed for recognizing voice commands may be reduced, which is advantageous in medical implants since the size of the implant may be decreased.

FIG. 695B shows a flow chart for a method for training a medical implant to recognize a voice command, according to some embodiments. The method 200 comprises receiving 210, by a medical implant, a first audio training phrase, when the medical implant is implanted in the body of the patient.

The method further comprises creating 220 a transfer function, the transfer function being based on the first audio training phrase, wherein the transfer function is configured to adjust the amplitude of at least one frequency of audio received at the medical device for enhancing audio received at the medical implant to facilitate detection of voice commands. Creating 220 a transfer function based on a first audio input phase when the implant has been implanted in a patient allows for specifically correcting the audio input phrase for noise and/or distortion caused by the patient's body specifically.

The creation of the transfer function may be based on training a machine learning model.

A purpose of the transfer function may be to adjust the audio input for distortions or noise specific to the body the implant has been implanted into. After the audio input has been adjusted, or enhanced, the audio input may be in a better condition for use in later steps of the method, such as for recognizing a command for the medical implant comprised in the audio input. In that way, there may be a two-step method for training the medical implant to recognize commands. Since the audio input has been adjusted or enhanced, the voice recognition of the command in the audio input may be easier, which may allow for using less processing power.

In some embodiments, the creating 220 a transfer function may further comprise to compare 221 the first audio training phrase with a stored audio phrase to determine a difference between them. Based on the difference, the transfer function may be created 222. In other words, the method may comprise creating a transfer function based on a difference between a stored audio phrase and the first audio phrase.

As an illustrative example only, the stored audio phrase may comprise a specific command or test phrase. When in a training session, a user of the implant or another person that the implant should be trained for, may speak the same specific command or test phrase. The command or test phrase may then be captured by the microphone of the implant, and transferred to the learning unit of the processor. The learning unit may then compare the received command or test phrase with the stored command or test phrase, and then, based on the difference(s), create a transfer function which takes the differences into account. The differences between the received command or test phrase and the stored command or test phrase may be indicative of a noise or distortion created by the body in which the implant has been implanted.

The method 200 may further comprise inputting 230 a second audio training phrase to the medical implant, the second audio training phrase comprising the voice command, the voice command comprising an instruction for the control of the medical implant.

The second audio training phrase may be used as input to the transfer function in order to create an enhanced audio training phrase. In this way, any noise or distortion created by the body may be alleviated by the transfer function, thus resulting in an enhanced audio training phrase. Thus, the method may further comprise using 240 the transfer function for generating an enhanced second audio training phrase in the medical implant.

The enhanced audio training phrase may then be associated 250 with the instruction for the control of the medical implant. That is, the method may comprise training a command unit to associate the second audio training phrase to a command for the medical device. The training may comprise training a machine learning model to associate enhanced audio training phrases with commands for the medical implant.

By first creating a transfer function, any following audio input may be enhanced by using the transfer function, and thus the associating of a second audio training phrase with a command may be simpler, i.e. less computationally intense, as the quality of the enhanced audio may be better that the originally audio received by the microphone of the implant. The method also allows for avoiding training the medical implant on distorted audio or audio with a lot of noise, thus improving the quality of the training.

FIG. 695C shows a flow chart for a method 300 for using voice commands to control a medical implant, according to some embodiments. In some examples, the voice commands have been learnt with the method described with reference to FIGS. 695A and 695B.

The method 300 comprises receiving 310 an audio command phrase for the medical device. The method further comprises applying 320 a transfer function to create an enhanced audio command phrase.

The method may further comprise to determine 320 a corresponding command for the medical based on the enhance audio command phrase, and send 340 the command to the medical device. The medical device may then execute 350 the command. By running the audio command phrase through the transfer function, the audio quality of the audio command phrase may be improved, thus allowing for an easier recognition of the corresponding command. This may make the recognition or determination of the command for the medical device less computationally intensive.

### Aspect 457: Controlling Energy Transfer at the Implant

Any of the implantable medical implants described herein are configured to wirelessly receive energy for powering or charging the implantable medical implant. When transferring energy to an implantable medical implant it is important to adequately control the energy transfer. If the energy transferred or received at the medical implant is excessive, it may harm the patient. For example, if the position of external device relative to the receiving unit changes during energy transfer, the energy transferred may also increase or decrease drastically. This situation could cause severe problems since the implant cannot “consume” the suddenly very high amount of supplied energy. Unused excessive energy must be absorbed in some way, resulting in the generation of heat, which is highly undesirable as it may harm the patient. Hence, if excessive energy is transferred from external device the receiving unit, the temperature of the implant will increase, which may damage the surrounding tissue or otherwise have a negative effect on body functions. It is therefore highly desirable to always supply the right amount of energy to an implanted medical device during operation. Similarly, if too much energy is received by the implant, there may be temperature increases which may harm the patient. It has thus been realized that controlling the energy transfer at the medical implant may be advantageous.

An embodiment of a system for transferring energy to an implantable medical device will now be described with reference to FIGS. 119a-h. Alternatively, “transferring energy” may be referred to as “charging”. A corresponding a method for wireless energy transfer from an external energy source located outside the patient to an internal energy receiver 305 located inside the patient is also provided.

The system comprises an external energy source, or a charger, and an internal energy receiver 305. The external energy source may be comprised in any of the external devices, i.e. devices arranged outside of the body of a patient, described herein. The internal energy receiver 305 is connected to an implantable medical device 300 for supplying received energy thereto. Internal energy receiver 305 may be configured to determine an accumulated amount of received energy; determine a current change in the received energy, determine a control signal reflecting the accumulated received energy and the change in the received energy, and controlling the energy transfer based on the control signal. As an alternative, the determination of the control signal may be omitted, and the controlling may be performed based on the accumulated amount of energy and the current change.

By “controlling the energy transfer” it may be meant or include adjusting the energy transfer efficiency, controlling switches affecting the energy transfer, controlling a part of the internal energy receiver, controlling a part of the external energy source, turning the energy transfer off completely, or any other way of affecting the energy transfer.

In one embodiment the external energy source or the internal energy receiver 305 may comprise an energy transfer controller for controlling the energy transfer. The energy transfer controller may be configured to determine the rate of change of the received energy and/or the accumulated amount of received energy, and adjust the energy transfer based on the determined parameters.

Advantageously, the energy transfer may be controlled or adjusted by the internal energy receiver 305, as the internal energy receiver 305 is capable of directly determining how much energy is received in the internal energy receiver and faster determine if there is a risk to the patient or the medical implant. Thus, the internal energy receiver 305 may be configured to determine an accumulated amount of transferred energy is determined by the internal energy receiver 305. The internal energy receiver 305 may alternatively or in combination, be configured to determine a current change in the energy transfer. Further, the internal energy receiver 305 may be configured to determine a control signal for controlling the energy transfer. The control signal may be used in the internal energy receiver 305 for adjusting the receiving of energy, or it may be transmitted to the external energy source, and the external energy source may be configured to adjust the transmitted energy based on the control signal. That is, the controlling of the energy transfer may be performed by the internal energy receiver 305.

In some examples, the controlling of the energy transfer may be performed by the external energy source.

In some examples, the internal energy receiver 305 is configured to measure, via a measuring unit, an accumulated energy received a period of time and/or to measure a current change in energy received, and to control the energy received based on the accumulated energy and/or the current change. In some examples, this may be performed using a PID regulator, which will be described in the following.

In some examples, the controller comprised in the internal energy receiver comprises a PID regulator. Such a PID regulator may be used to control the difference between a received voltage and a desired voltage level. The PID regulator may control a switch to signal to selectively de-tune the receiving coil of the internal energy receiver. Alternatively, or in combination, the PID may regulate the switch to modulate the power signal. The PID regulator may respond quickly to changes in the power levels and provides increased control over the pulse width modulation of the power signal.

A PID regulator may be used for controlling any energy transfer as discussed herein.

### Pulse Width Modulation (PWM)

In some embodiments, the energy is supplied from the primary coil to the secondary coil using energy pulses. The pulses are achieved using modulation techniques. For example, modulation (PWMT—Pulse width modulation technique) of the pulses may be created with a system that controls the power using a continuous square wave pulse signal with a constant frequency where the duty cycle of the pulses is varied or a system that controls power using a continuous square wave pulse train signal with both constant frequency and constant pulse with and thereby adjusting the duty cycle width of the train of pulses. The PWMT may be used to digitally vary the amount of power from the power amplifier that drives the transmitting coil. Thus, the amount of energy transferred from the primary coil to the secondary coil may be controlled.

In some examples, the energy is supplied using a pulse pattern. In those examples, the receiving unit 305 may be configured to receive transcutaneously transferred energy in pulses according to a pulse pattern, and the measurement unit may be configured to measure a parameter related to the pulse pattern. In some examples, the controller is configured to control the energy received (for example by a variable impedance or via switches as described below) in response to the pulse pattern deviating from a predefined pulse pattern.

In some examples, the energy transmitted may be varied by varying the width of the energy pulses and having constant frequency and constant amplitude. The pulse width is achieved with a modulation technique, (hereafter PWMT) (in the preferred embodiment many times per second), to control the amount of energy transferred from the external energy transmitting coil in the system to the implanted receiver. The PWMT is used to digitally vary the amount of power from a power amplifier that drives the transmitting coil. There are several different ways to achieve the PWMT to control the amount of output energy from the power amplifier to the transmitting coil. Generally, modulation of the pulse width may be created with a system that controls the power using a continuous square wave pulse signal with a constant frequency where the duty cycle of the pulses are varied or a system that controls power using a continuous square wave pulse train signal with both constant frequency and a constant pulse width and thereby adjusting the duty cycle width of the train of pulses. These two basic techniques as well as most modifications of them can be used to control the output power of the transmitting coil.

The transmission of wireless energy from the external energy transmitting device may be controlled by applying to the external energy transmitting device electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

Advantageously, the PWM embodiments described herein may be combined with any embodiment relating to controlling energy transfer to an implantable medica device, variable impedance, resonant circuit, NFMI, large coil, or any other implantable medical device being in any way configured to receive energy wirelessly, as described herein.

### Aspect 332: Variable Impedance

According to one embodiment described with reference to FIG. 119A-H, the controller 300 of the implantable system 10 comprises a receiving unit 305 or energy receiver 305 comprising a coil) configured for receiving transcutaneously transferred energy. The receiving unit 305 further comprises a measurement unit 194 configured to measure a parameter related to the energy received by the coil and a variable impedance electrically connected to the coil. The receiving unit 305 further comprises a switch 195a placed between the variable impedance and the coil for switching off the electrical connection between the variable impedance and the coil. The controller 300 is configured to control the variable impedance for varying the impedance and thereby tune the coil based on the measured parameter. The controller 300 is further configured to control the switch 195a for switching off the electrical connection between the variable impedance and the coil in response to the measured parameter exceeding a threshold value. As such, the coil can be tuned or switched off to reduce the amount of received energy if the amount of received energy becomes excessive.

The controller 300 may further be configured to vary the variable impedance in response to the measured parameter exceeding a threshold value. By varying the variable impedance, the tuning of the coil may be varied, thus affecting the resonant frequency of the receiving coil. In this way, the efficiency of the reception of energy may be varied.

The measurement unit is configured to measure a parameter related to the energy received by the coil over a time period and/or measure a parameter related to a change in energy received by the coil by for example measure the derivative of the received energy over time. The variable impedance is in one embodiment placed in series with the coil. In alternative embodiments it is however conceivable that the variable impedance is placed parallel to the coil.

The first switch is placed at a first end portion of the coil, and the receiving unit 305 further comprises a second switch placed at a second end portion of the coil, such that the coil can be completely disconnected from other portions of the implantable system 10. The receiving unit 305 is configured to receive transcutaneously transferred energy in pulses according to a pulse pattern. The measurement unit is in one embodiment configured to measure a parameter related to the pulse pattern. The controller 300 is configured to control the variable impedance in response to the pulse pattern deviating from a predefined pulse pattern. The controller 300 is configured to control the switch for switching off the electrical connection between the variable impedance and the coil in response to the pulse pattern deviating from a predefined pulse pattern. The measurement unit is configured to measure a temperature in the implantable system 10 or in the body of the patient, and the controller 300 is configured to control the first and second switch in response to the measured temperature.

The variable impedance may comprise a resistor and a capacitor and/or a resistor and an inductor and/or an inductor and a capacitor. The variable impedance may comprise a digitally tuned capacitor or a digital potentiometer. The variable impedance may comprise a variable inductor. The first and second switch comprises a semiconductor, such as a MOSFET. The variation of the impedance is configured to lower the active power that is received by the receiving unit. The variable impedance, the first and second switch and the measurement unit may be connected to the communication unit/controller 300 and the receiving unit 305 is connected to an energy storage unit 40 such that the energy storage unit 40 can store energy received by the receiving unit 305.

### Aspect 459: Resonant Circuit

FIG. 697A schematically illustrates a system 500 comprising an external unit 510 and an implantable medical device 505. The external unit 510 is adapted to be arranged outside of a body. The implantable medical device 505 is adapted to be implanted into a body of a patient. The external unit 510 comprises a transmitting circuit 512, a transmitting coil 514 and electrical connections 502. The electrical connections 502 electrically connect the transmitting circuit 512 with the transmitting coil 514. The electrical connections 502 may, for example, be wires or any electrically conducting material, or they may be wireless electrical connections. The electrical connections 502 may have intrinsic electrical resistance.

The implantable medical device 505 comprises an energy consuming part 528. The implantable medical device 505 further comprises receiving units 530 for receiving transcutaneously transferred energy, wherein the receiving unit 530 is configured to transfer the received energy to the energy consuming part 528. In FIG. 65P, three receiving units 530 are illustrated. However, the medical device 505 may comprise any number of receiving units 530. The medical device 505 preferably comprises two or more receiving units 530. Each receiving unit 530 comprises a receiving circuit 522, an impedance unit 526 and a receiving coil, or coil, 524, and electrical connections 502. The electrical connections 502 in the receiving unit 530 electrically connect the receiving circuit 522 to the impedance unit 526 and to the coil 524. Electrical connections 502 electrically connect the receiving unit 530 to the energy consuming part 528. The receiving unit 530 may be directly connected to the energy consuming part 528.

The receiving unit 530 may be connected to intermediate circuits, wherein the intermediate circuits are connected to the energy consuming part 528. The intermediate circuits may process the output from the receiving unit 530 and prepare it for the energy consuming part 528. The intermediate circuit may comprise amplifiers, switches, filtering, modulators, other signal transformers, or a combination thereof.

In FIG. 65P, the impedance units 526 are connected in parallel to the coil 524. The impedance units 526 and the respective coil 524 may instead be connected in series, partially in series and partially in parallel, or in any other way. I FIG. 65P, the impedance units 526 are capacitors. The impedance unit 526 may consist of inductors, capacitors, capacitors and resistances, inductors and resistances, or a mixture thereof. The impedance unit 526 may have a variable impedance.

The implantable medical device may further comprise a measurement unit 521 and a controller 520. The measurement unit 521 may be configured to measure a parameter related to energy transfer from the external unit 510 to the implantable medical device 505. The controller 520 may be configured to control the subcutaneously received energy to the energy consuming part 528. The controller 520 may be configured to control the subcutaneously received energy based on the parameter measured by the measurement unit 521. The controller 520 may control the impedance units 526. The controller 520 may control a variable impedance of the impedance unit 526.

The implantable medical device 505 and the external unit 510 are electrically coupled. The transmitting circuit 512 generates an alternating current in the transmitting coil 514. The alternating current of the transmitting coil 514 induces a current in the coil(s) 524. The receiving unit 530 is configured to receive transcutaneously transferred energy from the external unit 505 via the coil 524. One external unit 510 may transfer energy to many receiving units 530 having a respective coil 524. The inductance of the coil 524 and the impedance of the corresponding impedance unit 526 contributes to a resonance frequency of the receiving unit 530. The inductance of the coils 524 and/or the impedance of the corresponding impedance unit 526 may differ in size between the respective receiving units 530. This may cause receiving units 530 to have different resonance frequencies in relation to each other. A variable impedance of the impedance unit 526 may allow the resonance frequencies of the receiving unit 530 to be tuned. The controller 520 may be able to tune the resonance frequency of each of the receiving units 530 individually by controlling the respective impedance unit 526. The receiving unit 530 may transfer different amounts of energy to the energy consuming device 528 depending on the frequency of an alternating magnetic field generated by the external device 510 and the resonance frequency of the receiving unit 530. By having different resonance frequencies for receiving units 530, a better energy transfer efficiency of the implantable medical device 505 may be obtained. Each receiving unit 530 may be designed to, or be fined tuned to, have the resonance frequency adapted to different frequencies of the external unit 510. By having different resonance frequencies of the receiving units 530, different external units 510 may be used, which is illustrated in FIG. 65Q.

FIG. 697B schematically illustrates a system 540 comprising a second external unit 511 and an implantable medical device 545. The second external unit 511 comprises several transmitting units 509, wherein each transmitting unit 509 is similar to the external unit 510 illustrated in FIG. 65P, described above. The implantable medical device 545 illustrated in FIG. 697B comprises an energy consuming part 528 and receiving units 530. The energy consuming part 528 of FIG. 697B is similar to the energy consuming part 528 of FIG. 65P, described above. The receiving units 530 of FIG. 697B are similar to the receiving units 530 of FIG. 697A, described above. The implantable medical device 545 of FIG. 697B may further comprise a measurement unit and/or a controller similar to the measurement unit 521 and the controller 520 of FIG. 65P, described above.

FIG. 697B illustrates an implantable medical device 545 comprising two receiving units 530.

As mentioned above, there may be any number of receiving units 530 in the implantable medical device 545. As mentioned above, the receiving units 530 are connected to the energy consuming part 528, directly or via an intermediate circuit. The receiving units 530 are adapted to receive transcutaneously transferred energy from the external device 511. The external device 511 is adapted to transmit energy to the receiving unit 530 via the transmitting units 509 in the external device 511.

The external unit 511 of FIG. 697B illustrates several transmitting units 509. The transmitting units 509 may all be comprised in one device, in separate devices, or a combination thereof. Each transmitting unit 509 may be adapted to send energy on a separate frequency. The transmitting units 509 illustrated in FIG. 697B may be configured to transmit different frequencies. A transmitting unit 509 may be comprised in any external device or remote control described herein, a charging device, such as a smartphone, a qi charger, wireless charging pad, any device comprising a coil configured to send out energy, or any device configured to produce an oscillating a magnetic field. Each receiving unit 530 may be adapted to receive energy from a different transmitting unit 509 by having its resonance frequency configured to match the frequency of the transmitting unit 509. The transmitting units 509 could transmit sequentially, simultaneously, or have one or more transmissions partially overlap.

An advantage of multiple transmitting units 509 is that a better energy transfer efficiency of the implantable medical device 545 may be obtained. Each receiving unit 530 may be tuned to receive energy of a specific frequency of a corresponding transmitting unit 509, so that the receiving units 530 could be charged by their respective transmitting unit 509. Each receiving unit 530 may receive a respective transmitted energy sequentially, simultaneously, and/or independently of the other receiving units 530. An advantageous transmitted energy for a receiving unit 530 may be energy with the frequency of the resonance frequency of the respective receiving unit 530, energy with a frequency within a symmetric or nonsymmetric range around the resonance frequency of the respective receiving unit 530, or energy with a frequency that is at an offset from the resonance frequency of the respective receiving unit 530.

Each receiving unit 530 comprises a coil 524 and a resonance frequency. The resonance frequency is a function of the coil 524. Instead of a coil 524 and one resonance frequency, a part of a coil 524 may contribute to a resonance frequency, meaning that a coil 524 may have several resonance frequencies. This is illustrated in FIG. 697C.

FIG. 697C schematically illustrates an implantable medical device 565. The implantable medical device 565 is adapted to be implanted into a body of a patient. The implantable medical device 565 comprises an energy consuming part 528, similar to the energy consuming parts of FIGS. 697A and 697B, described above. The implantable medical device 565 of FIG. 697C may further comprise a measurement unit and/or a controller similar to the measurement unit 521 and the controller 520 of FIG. 697A, described above. The medical device 565 may be configured to receive energy from an external unit, such as the external unit 510 of FIG. 697A and/or the external unit 511 of FIG. 697B.

The implantable medical device 565 further comprises a receiving unit 535 for receiving transcutaneously transferred energy, wherein the receiving unit 535 is configured to transfer the received energy to the energy consuming part 528. The receiving unit 535 comprises a receiving circuit 523. The receiving unit 535 comprises a receiver coil, wherein the receiver coil comprises a coil with one or more center taps, a multitude of coils in parallel, or a combination thereof. Center taps do not have to be positioned in the center of a coil. Parts of a receiver coil are coil portions 525. The receiving unit 535 of FIG. 697C further comprises impedance units 526, similar to the impedance units 526 of FIG. 697A, described above. The receiving unit 535 of FIG. 697C further comprises electrical connections 502, similar to the electrical connections 502 of FIG. 697A, described above. The electrical connections 502 connect the receiving unit 535 to the energy consuming part 528. As mentioned for the receiving unit 530 of FIG. 697A, the receiving unit 535 of FIG. 697C may be connected directly to the energy consuming part 528 or connected first to intermediate circuits, wherein the intermediate circuits are connected to the energy consuming part 528. Intermediate circuits may be the same as described for FIG. 697A above.

The electrical connections 502 in the receiving unit 535 connect the receiving circuit 523 to the impedance units 526 and the coil portions 525 so that each impedance unit 526 is connected to a respective coil portion 525. The impedance unit 526 and the respective coil portions 525 form a receiving portion. The receiving portions may be seen as akin to the receiving units 530 of FIG. 697A, described above. FIG. 697C illustrates each impedance unit 526 being connected in parallel to the respective coil portion 525. The impedance unit 526 may be connected in series, or partially in series and partially in parallel, with the respective coil portion 525. The receiving circuit 523 is connected in parallel to the impedance unit 526. The receiving circuit 523 may be connected in parallel to a portion of the impedance unit 526. The receiving circuit 523 may be connected in series with the entire, or a portion of the, impedance unit 526.

The inductance of the coil portion 525 and the impedance of the corresponding impedance unit 526 contribute to a resonance frequency of the corresponding receiving portion. The inductance of the coil portions 525 and/or the impedance of the corresponding impedance unit 526 may differ in size between the respective receiving portions. This may cause receiving portions to have different resonance frequencies in relation to each other. A variable impedance of the impedance unit 526 may be individually controlled by a controller to change the resonance frequencies of the respective receiving portions. Each receiving portion may transfer different amounts of energy to the energy consuming device 528 depending on the resonance frequency of the receiving portion and the frequency of the transcutaneous transferred energy. By having different resonance frequencies of the receiving portions, a better energy transfer efficiency of the implantable medical device 565 may be obtained. Consecutive, sequential, or independent charging may be performed, where each receiving portion receives energy of different frequencies. Each receiving portion may have a resonance frequency adapted to different transcutaneously transferred energy frequencies, from one or more external units.

Advantages of having coil portions 525 include that it may reduce the required amount of coils and the amount of material needed. FIG. 697C illustrates sequential coil portions 525, where the coil portions 525 are adjacent but not overlapping. Coil portions 525 may overlap, be separate, or be partially overlapping segments of the receiving coil. This is illustrated in FIG. 697D.

FIG. 697D illustrates an implantable medical device 575. The implantable medical device 575 comprises an energy consuming part 528, a receiving circuit 523, and impedance units 526, similar to the energy consuming part 528, receiving circuit 523, and impedance units 526 of FIG. 697C, respectively. The implantable medical device of FIG. 65S may further comprise a measurement unit and/or a controller similar to the measurement unit 521 and the controller 520 of FIG. 697A, described above. Similar to the receiving unit 530 of FIG. 697A, the receiving unit 575 of FIG. 697D may be connected directly to the energy consuming part 528 or connected first to intermediate circuits, wherein the intermediate circuits are connected to the energy consuming part 528. Intermediate circuits may be the same as described for FIG. 697A above. The medical device 575 of FIG. 697D may be configured to receive energy from an external unit, such as the external unit 510 of FIG. 697A and/or the external unit 511 of FIG. 697B.

The implantable medical device 575 further comprises coil portions 525, similar to the coil portions 525 of FIG. 697C, described above. FIG. 697D illustrates coil portions 525 that are overlapping with each other. Each coil portion 525 is connected to the respective impedance unit 526, as mentioned for the coil portions 525 and the impedance units 526 of FIG. 697C. An advantage of having overlapping coil portions 525 is that shorter or fewer coils may be used. Overlapping coil portions allow for larger inductances of the coil portions for a set receiver coil. Overlapping coil portions allows for a better energy transfer efficiency.

FIG. 697E illustrates an implantable medical device 585. The implantable medical device 585 comprises an energy consuming part 528, similar to the energy consuming part 528 of any of FIGS. 697A-D. The implantable medical device 585 of FIG. 697E further comprise a first receiving unit 530, similar to the receiving unit 530 of FIGS. 697A and 697B. The implantable medical device 585 of FIG. 697E further comprise a second receiving unit 535, similar to the receiving unit 535 of any of FIGS. 697C and 697D. The first receiving unit 530 and the second receiving unit 535 are electrically connected to the energy consuming part 528 by electrical connections 502. As mentioned for the receiving unit 530 of FIG. 697A, the receiving unit 585 of FIG. 697E may be connected directly to the energy consuming part 528 or connected first to intermediate circuits, wherein the intermediate circuits are connected to the energy consuming part 528. Intermediate circuits may be the same as described for FIG. 697A above.

FIG. 697E illustrates a implantable medical device 585 comprising one first receiving unit 530 and one second receiving unit 535. The implantable medical device 585 may comprise more than one first receiving unit 530. The implantable medical device 585 may comprise more than one second receiving unit 535. A mix of first receiving units 530 and second receiving units 535 may allow the implantable medical device 585 to be compact and customizable. The implantable medical device 585 may comprise more than one second receiving unit 535 and no first receiving units 530.

The implantable medical device 585 of FIG. 697E may further comprise a measurement unit and/or a controller similar to the measurement unit 521 and the controller 520 of FIG. 697A. The medical device 585 may be configured to receive energy from an external unit, such as the external unit 510 of FIG. 697A and/or the external unit 511 of FIG. 697B.

In some examples, a coil comprised in the receiving unit 530 may comprise a plurality of windings. The plurality of windings may be connected to a respective variable impedance (as described above). An internal controller may control each of the variable impedances individually, thus providing for adjusting the resonant frequency of each of the windings separately. For examples, the secondary coil may comprise a first and a second winding, each connected to a respective variable impedance.

### Large Coil

A system for wirelessly charging an implantable medical implant, when implanted in a body of a patient is provided. The system comprises an internal energy receiver comprising a secondary coil, the internal energy receiver being connected to the implantable medical implant and an external energy transmitter comprising a primary coil for wirelessly transmitting energy to the internal energy receiver via the secondary coil. The diameter of the primary coil is larger than a diameter of the secondary coil.

According to embodiments described with reference to FIG. 119A-H, the controller 300 of the implantable system 10 comprises a receiving unit 305 or internal energy receiver 305 comprising a secondary coil 192 (specifically shown in FIG. 65B′) configured for receiving transcutaneously transferred energy. The implantable system may receive the energy from an external device (also called an external energy transmitter), the external device being arranged outside of the body of the patient. The external device may comprise a primary coil for inducing a current in the coil 192 of the energy receiver 304 for wirelessly transfer energy to the receiving unit 305.

According to some embodiments, the primary coil is larger than the coil 192. By having the primary coil being larger than the secondary coil 192, the energy transmission may be improved. By having a diameter of the primary coil being larger than a diameter of the secondary coil, the wireless charging may be improved. For example, in previous wireless charging solution, there is a need for a great precision of arrangement of the secondary coil in relation to the primary coil. By having a larger diameter of the secondary coil, the need for precision may be reduced. Furthermore, having a larger primary coil wirelessly transmitting energy to a small secondary coil may provide for an improved energy transfer efficiency.

The implantable medical device may further comprise an internal controller connected to the internal energy receiver, for controlling the amount of energy received by the internal energy receiver. In some examples, the internal energy receiver further comprises a measurement unit for measuring a parameter related to the implantable medical implant or the body of the patient. The controller may be configured to measure the accumulated energy received by the internal energy receiver over a period of time and to measure a current change in energy received, and to control the energy received based on the accumulated energy and the current change. In some examples, the controlled comprises a Proportional-Integral-Derivative, PID, regulator for controlling the received energy.

The implantable medical device may comprise a variable impedance and/or a switch as described above.

With regards to the primary coil, the diameter of the primary coil may be more than 0.5 cm, more than 10 cm, more than 15 cm, more than 20 cm, more than 30 cm, or more than 50 cm. Alternatively, or in combination, the area of the primary coil is more than 0.5 cm2, more than 2 cm2, more than 10 cm2, more than 100 cm2, more than 300 cm2, more than 500 cm2, or more than 800 cm2.

Advantageously, any of the embodiments relating to wireless charging, for example, controlling energy transfer, PID regulation, variable impedance, large coil, and emergency backup function, among others, may be combined with any embodiment related to energy transfer described herein, for example Aspects 432, 433, 434, for an increased energy transfer safety mechanism.

### Emergency Backup Function

Another risk associated with an energized implantable medical device is that the implantable medical device's battery or energy storage is depleted and thus unable to energize the implantable medical device. Further, there is a risk that the internal energy receiver malfunctions, also resulting in a malfunction of the powering of the implantable medical device.

Thus, there is provided a safety mechanism that may be advantageously combined with any embodiment or aspect relating to an energized implantable medical implant described herein.

FIG. 65V shows a schematic illustration of an implantable medical device 100. The implantable medical device 100 may have an active portion 112 and an internal energy receiver 395. The active portion 112 may, for example, be configured to treat, monitor or perform a function of a body of a patient. The implantable medical implant 100 may comprise or be connected to a backup system 113, the backup system 113 being adapted to perform a backup function related to the active portion 112 of the implantable medical device 100.

In some examples, the backup function relates to switching a function of the active portion 112 off. The backup function may be any function relating to the function of the active portion, such as, but not limited to: opening an artificial sphincter, stopping a stretching a stomach portion, or stopping a stimulation of tissue. In some examples, the backup system is configured to reverse a function of the medical devices. For example, if the implanted medical device is used to constrict the urethra of a patient having urinary incontinence, the user must naturally be capable of opening said constriction, in order to perform urination, even if the implantable medical device 100 is malfunctioning.

The backup system 113 may, for example, comprise a backup energy receiver 114 to receive energy from an external device (such as any of the external devices or remote controls described herein), or to perform a function of the active portion. The backup energy receiver 114 may be adapted to receive wirelessly transferred energy from an external device (which may also be referred to as an external energy transmitter). To this end, the backup energy receiver may comprise a second secondary coil for receiving such energy. For example, in a case where the implantable medical device 100 malfunctions, an external device may wirelessly transfer energy to the backup energy receiver. The backup energy receiver 114 may receive the wirelessly transferred energy and the received energy may be used by the backup system 113 to perform the backup function.

In some examples, the function of the backup system 113 is to transfer the energy received via the backup energy receiver 114 for powering the medical device 100, or it may be used to charge a battery or accumulator of the medical device 100.

In some examples, the backup system 113 may use a battery or energy storage used by the active portion 112.

The backup function may be triggered by an external device 320′″. The external device may be any external device or remote control as described herein. The external device 320′″ may be adapted to wirelessly transfer energy to the backup system, and/or be configured to trigger the backup function of the backup system 113. The backup function may thus comprise an backup internal communications unit 115 for receiving a command from the external device, and be configured to execute the received command.

In some examples, the backup function may be triggered by an error detected by a measuring unit or a controller comprised in the medical implant. Such an error may, for example, be detected by a pressure being too high or too low, a temperature being too high or low, a battery charge status being too low, a measurement value deviating from a predetermined interval, or something else.

In other examples, a malfunction of the implantable medical device 100 may relate to the programming of the implantable medical device. In that case, the backup function of the backup system may be to re-program the malfunctioning program of the implantable medical device 100. The re-programming may be performed using any of the methods described herein.

In some examples, the backup energy receiver 114 comprises a passive or active RFID circuit adapted to be powered by the external device. In some examples, the backup energy receiver 114 comprises an NFMI energy receiver adapted to receive energy from the external device. The backup energy receiver 114 and the backup internal communication unit 115 may in some examples be comprised in the same unit, for example, in the cases where energy transmission and wireless communication may be performed using the same hardware.

### Aspect 456: NFMI Communication and Wireless Energy Transfer

Any one of the medical devices described herein which utilize wireless communication in any way may be comprised in a system for communicating information from or to an implantable medical device, wherein the implantable medical device is implanted in a body of a patient. The system may comprise an internal communications unit comprised in or connected to the implantable medical device, and an external communications unit, wherein the internal communications unit and the external communications units are configured to send or receive data using near-field magnetic induction.

NFMI is a short-range wireless technology that communicates using a tightly coupled magnetic field. By the term NFMI it may be meant a short range wireless physical layer using low-power and non-propagating magnetic field. NFMI systems are designed to contain transmission energy within the localized magnetic field, and the magnetic field energy resonates around the communication system, but does not radiate into free space. The power density of near-field transmissions is restrictive and attenuates or rolls off at a rate proportional to the inverse of the range to the sixth power (1/r6) or −60 dB per decade. Thus, NFMI in the typical use only has a reach of around 1.5 to 2 meters.

NFMI signal can penetrate through human body tissue with low absorption rate. For example, the specific absorption rate (SAR) may be 100 times lower than Bluetooth. It has been realized that NFMI has a communication range through body tissue of for example 50 cm, which thus makes it advantageous to use for medical implants, as compared to RF communication which is disturbed by passing though body tissue. Thus, NFMI allows for communication with implants implanted also implanted deeper in the body.

Since NFMI has such a short rage, the possibility of an adversary to eavesdrop on communication with an implant, or to hack an implant form a distance is greatly reduced, as any adversary must be very close to the implant.

FIG. 65U shows an example of a system 600 using NFMI communication between an external communications unit 601 and an implantable medical device 603. The communications unit 602 is configured to communicate with the implantable medical device 603 through the skin 624 of the patient using NFMI communication. The implantable medical device comprises an internal communications unit 610 (which may alternatively be referred to as a receiving unit 305 in other embodiments) and an active portion 612. The active portion 612 may be configured to monitor, treat or perform a function of a body of a patient, and may be any medical device or medical implant described herein. The communications unit 601 may be comprised in any external device described herein.

The external communications unit 601 comprises an external coil 604 connected to an external NFMI transceiver 606. The external NFMI transceiver 606 which may comprise an NFMI transmitter chip. The external coil 604 and the external NFMI transceiver are configured to modulate a magnetic field for sending data and/or energy to the implantable medical device 603. The external NFMI may further comprise a capacitor for tuning.

In turn, the internal communications unit 610 may comprise an internal coil 614 and an internal NFMI transceiver 616. To receive data, the magnetic field modulated by the external coil 604 induces a voltage on the internal coil 614, which may be measured by the internal NFMI transceiver 616 and be decoded at the internal NFMI transceiver or at another part of the implantable medical device 603. The NFMI transceiver 616 may comprise an NFMI receiver chip. The NFMI receiver chip may comprise a tunable resistor and capacitor tank. Both of the tunable capacitance and resistance may vary within a certain range to automatically compensate the detuning of NFMI antennas.

It will be appreciated that a similar method may be used for sending data from the implantable medical device via the internal communications unit 616 to the communications unit 601 via the external communications unit 606. In that examples, the internal communications unit may comprise an NFMI transmitter chip similar to the NFMI transmitter chip comprised in the external device, and the external NFMI transceiver may comprise an NFMI receiver chip similar to the NFMI receiver chip comprised in the internal NFMI transceiver, connected to a respective coil for transmitting and/or receiving data.

Modulation schemes such as amplitude modulation, phase modulation and frequency modulation typically used in RF communications may be used in NFMI communication.

In some embodiments, the active portion is not a pacemaker, hearing aid or a neurostimulation implant.

The internal communications unit is adapted to be implanted at a tissue depth of at least 8 or 10 cm. For example, the internal communications unit may be adapted to be implanted in an abdomen of a patient.

Thus, any internal wireless communication unit comprised in an implant described herein may use NFMI to communicate with an external device. For example, for transmitting data, receiving data, receiving new programming or changes to the software of the implant and/or receiving control commands. The short rage of NFMI and the tissue depth at which NFMI may be used, makes it advantageous to use for any communication between an external device, such as a patient EID 320″, a patient remote device 320′″, a HCP EID, a HCP remote device, and an implantable medica device.

While the communications security between an implant and an external device is improved by the use of NFMI (as compared to RF communication), the information security may advantageously be combined with any encryption, data integrity checks or the like described herein.

For example, the internal communications unit may be configured to encrypt any data to be transmitted to the external communications unit, and the external communications unit may be configured to receive the data transmitted from the internal communications unit. In some examples, the external communications unit may be further configured to transmit that data to a server.

In a more specific example, NFMI may be used for wireless communication between an implant and a patient external interrogation device, patient EID, as described herein.

In some examples the external communications unit is configured to transmit a control command to the internal communications unit, and the internal communications unit is configured to transmit the control command to the implantable medical device. The control command may cause the implantable medical device to perform an action. The internal communications unit may, for example, be configured to transmit data, the data relating to a function of the implantable medical device or a measurement obtained by the implant.

The magnetic field may in addition to or as an alternative be used for charging or powering the medical implant. The use of NFMI for changing is an alternative or addition to any wireless charging of a medical implant described herein. In those cases, the internal communications unit is configured to store the received energy in a battery or similar, or to directly forward the received energy to the active portion 612 or another energy consuming part of the implantable medical device 603.

Using NFMI for charging a medical implant also has the advantage, compared to previous methods of charging an implant, that it is not heavily affected by passing through body tissue. For example, with the use of NFMI for charging, an implant at a tissue depth of 8 and up to 13 cm or more may be charged. This allows for practically charging an implant in almost any part of a body.

Advantageously, the NFMI communication system disclosed herein may be combined with any of aspects 250, 252, 255 and 284, and any of the embodiments described herein relating to wireless energy transfer using a coil.

According to one example, the system further comprises a second internal communications unit and a second external communications unit, wherein the second internal communications unit is adapted to receive and transmit data using a short range communications technology, and the second external communications unit is adapted to receive and transmit data using a short range communications technology, the short range communications technology having a shorter maximum range than NFMI. In one example, the short range communications technology is NFC, and the second internal communication unit comprises an NFC transceiver and the second external communication unit comprises an interrogation device for transmitting data to and from the RFID transceiver. By having these second internal and external communications unit, the implant may require a second authentication based on that the external communication unit is close to the implant, for example close enough to interrogate the NCF transceiver. Thus, it may be verified that the second external communication device is indeed close to the patient's body.

### A: Pop-Rivet—Fixation and Housing of Control Units/Controllers and Operation Devices

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Embodiments of implantable devices for fixation and housing of implantable control units/controllers for controlling and communicating with at least one powered/operable implant, as well as for fixation and housing of operation devices for operating elements of powered/operable implantable medical device, will be described in the following. Such implantable devices for fixation and housing may be referred to as remote units or implantable energized medical devices.

In the following a detailed description of embodiments will be given with reference to the accompanying drawings. It will be appreciated that the drawings are for illustration only and are not in any way restricting the scope of the invention. Thus, any references to directions, such as “up” or “down”, are only referring to the directions shown in the figures. It should be noted that the features having the same reference numerals have the same function, a feature in one embodiment could thus be exchanged for a feature from another embodiment having the same reference numeral unless clearly contradictory. The descriptions of the features having the same reference numerals should thus be seen as complementing each other in describing the fundamental idea of the feature and thereby showing the features' versatility.

The different aspects or any part of an aspect or different embodiments or any part of an embodiment may all be combined in any possible way. Any method or any step of method may be seen also as an apparatus description, as well as, any apparatus embodiment, aspect or part of aspect or part of embodiment may be seen as a method description and all may be combined in any possible way down to the smallest detail. Any detailed description should be interpreted in its broadest outline as a general summary description, and please note that any embodiment or part of embodiment as well as any method or part of method could be combined in any way. All examples herein should be seen as part of the general description and therefore possible to combine in any way in general terms.

It is important to note that although the implantable energized medical device is disclosed herein as having a third cross-sectional area being smaller than a first cross-sectional area, this feature is not essential. The third cross-sectional area may be equal to or larger than the first cross-sectional area.

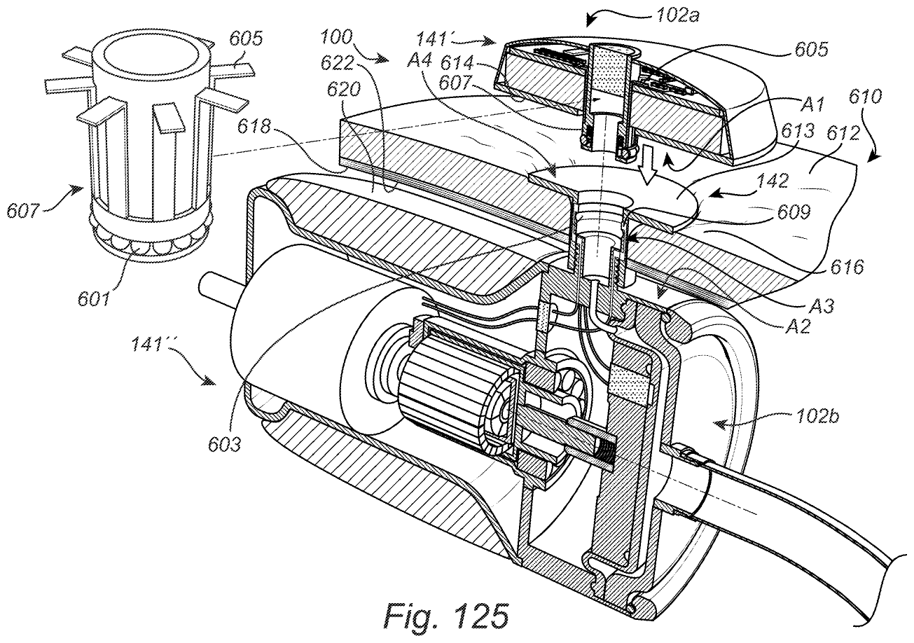

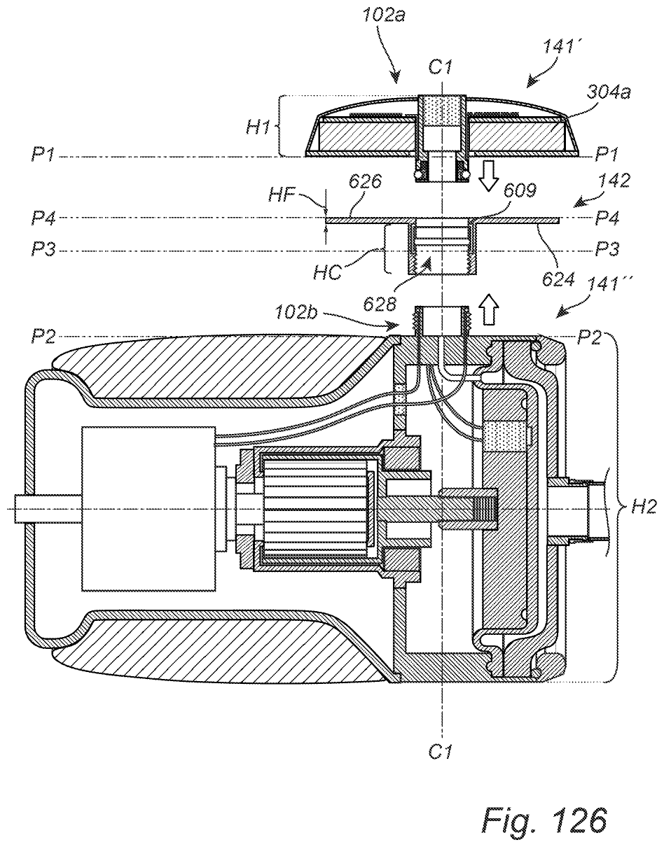

FIGS. 125 and 126 show an embodiment of an implantable energized medical device 100, which may be referred to as a remote unit in other parts of the present disclosure. The device 100 is configured to be held in position by a tissue portion 610 of a patient. The device 100 comprises a first portion 141′ configured to be placed on a first side 612 of the tissue portion 610, the first portion 141′ having a first cross-sectional area A1 in a first plane P1 and comprising a first surface 614 configured to face a first tissue surface 616 of the first side 612 of the tissue portion 610. The device 100 further comprises a second portion 141″ configured to be placed on a second side 618 of the tissue portion 610, the second side 618 opposing the first side 612, the second portion 141″ having a second cross-sectional area A2 in a second plane P2 and comprising a second surface 620 configured to engage a second tissue surface 622 of the second side 618 of the tissue portion 610. The device 100 further comprises a connecting portion 142 configured to be placed through a hole in the tissue portion 610 extending between the first and second sides 612, 618 of the tissue portion 610. The connecting portion 142 here has a third cross-sectional area A3 in a third plane P3 and a fourth cross-sectional area A4 in a fourth plane P4 and a third surface 624 configured to engage the first tissue surface 616 of the first side 612 of the tissue portion 610. The connecting portion 142 is configured to connect the first portion 141′ to the second portion 141″.

The connecting portion 142 thus has a portion being sized and shaped to fit through the hole in the tissue portion 610, such portion having the third cross-sectional area A3. Furthermore, the connecting portion 142 may have another portion being sized and shaped to not fit through the hole in the tissue portion 610, such portion having the fourth cross-sectional area A4. Likewise, the second portion 141″ may have a portion being sized and shaped to not fit through the hole in the tissue portion 610, such portion having the second cross-sectional area A2. Thus, the connecting portion 142 may cooperate with the second portion 141″ to keep the device in place in the hole of the tissue portion 610.

In the embodiment illustrated in FIG. 125, the first portion 141′ is configured to detachably connect, i.e. reversibly connect to the connecting portion 142 by a mechanical and/or magnetic mechanism. In the illustrated embodiment, a mechanic mechanism is used, wherein one or several spring-loaded spherical elements 601 lock in place in a groove 603 of the connecting portion 142 when the first portion 141′ is inserted into the connecting portion 142. Other locking mechanisms are envisioned, including corresponding threads and grooves, self-locking elements, and twist and lock fittings.

The device 100 is configured such that, when implanted, the first portion 141′ will be placed closer to an outside of the patient than the second portion 141″. Furthermore, in some implantation procedures the device 100 may be implanted such that space will be available beyond the second portion, i.e. beyond the second side 618 of the tissue portion 610, whereas there may not be as much space on the first side 612 of the tissue portion. Furthermore, tissue and/or skin may exert a force on the first portion 141″ towards the tissue portion 610 and provide for that the second portion 141″ does not travel through the hole in the tissue portion towards the first side 612 of the tissue portion. Thus, it is preferably if the device 100 is primarily configured to prevent the first portion 141″ from travelling through the hole in the tissue portion 612 towards the second side 618 of the tissue portion 610.

The first portion 141′ may further comprise one or several connections 605 for transferring energy and/or communication signals to the second portion 141″ via the connecting portion 142. The connections 605 in the illustrated embodiment are symmetrically arranged around a circumference of a protrusion 607 of the first portion 141′ and are arranged to engage with a corresponding connection 609 arranged at an inner surface of the connecting portion 142. The protrusion 607 may extend in a central extension C1 of the central portion 142. The second portion 141″ may also comprise one or several connections, which may be similarly arranged and configured as the connections 605 of the first portion 141′. For example, the one or several connections may engage with the connection 609 of the connecting portion 142 to receive energy and/or communication signals from the first portion 141′. Although the protrusion 607 is illustrated separately in FIG. 125, it is to be understood that the protrusion 607 may be formed as one integral unit with the first portion 141′.

Other arrangements of connections are envisioned, such as asymmetrically arranged connections around the circumference of the protrusion 607. It is also envisioned that one or several connections may be arranged on the first surface 614 of the first portion 141′, wherein the connections are arranged to engage with corresponding connections arranged on the opposing surface 613 of the connecting portion.

Such connections on the opposing surface 613 may cover a relatively large area as compared to the connection 609, thus allowing a larger area of contact and a higher rate and/or signal strength of energy and/or communication signal transfer. Furthermore, it is envisioned that a physical connection between the first portion 141′, connecting portion 142 and second portion 141″ may be replaced or accompanied by a wireless arrangement, as described further in other parts of the present disclosure.

Any of the first surface 614 of the first portion 141′, the second surface 620 of the second portion 141′, the third surface 624 of the connecting portion 142, and an opposing surface 613 of the connecting portion 142, may be provided with at least one of ribs, barbs, hooks, a friction enhancing surface treatment, and a friction enhancing material, to facilitate the device 100 being held in position by the tissue portion, and/or to facilitate that the different parts of the device are held in mutual position.

The opposing surface 613 of the connecting portion 142 and the first surface 614 of the first portion 141′ may provide, fully or partly, a connection mechanism to detachably connect the first portion 141′ to the connecting portion 142. Such connection mechanisms have been described previously in the presented disclosure and can be arranged on one or both of the opposing surface 613 and the first surface 614 and will not be further described here.

The opposing surface 613 may be provided with a recess configured to house at least part of the first portion 141′. In particular, such recess may be configured to receive at least a portion of the first portion 141′, including the first surface 614. Similarly, the first surface 614 may be provided with a recess configured to house at least part of the connecting portion 142. In particular, such recess may be configured to receive at least a portion of the connecting portion 142, and in some embodiments such recess may be configured to receive at least one protruding element to at least partially enclose at least one protruding element or flange.

In the illustrated embodiment, the first portion 141′ comprises a first energy storage unit 304a and a controller 102a comprising one or several processing units connected to the first energy storage unit 304a. The first energy storage unit 304a may be rechargeable by wireless transfer of energy. In some embodiments, the first energy storage unit 304a may be non-rechargeable. Upon reaching the life-time end of such first energy storage, a replacement first portion comprising a new first energy storage unit may simply be swapped in place for the first portion having the depleted first energy storage unit. The second portion 141″ may further comprise a controller 102b comprising one or several processing units.

As will be described in other parts of the present disclosure, the first portion 141′ and the second portion 141″ may comprise one or several functional parts, such as receivers, transmitters, transceivers, control units, processing units, sensors, energy storage units, sensors, etc.

The device 100 may be non-inflatable.

The second portion 141″ in the illustrated embodiment comprises a pump as described in conjunction with FIGS. 137gg-137kk, 137mm, 137nn, 137pp-137rr. However, it is to be understood that other embodiments of the second portion 141″ are able to be connected to the first portion 141′ via the connecting portion 142, such as second portions 141″ comprising a motor for providing mechanical work without the use of fluids. Furthermore, although the connecting portion 142 is illustrated in FIG. 125 as a separate unit, the connecting portion 142 may form part of the second portion 141″.

The first portion 141′ may be detachably connected to at least one of the connecting portion 142 and the second portion 141″.

As can be seen in FIG. 126, the first, second, third and fourth planes P1, P2, P3 and P4, are parallel to each other. Furthermore, in the illustrated embodiment, the third cross-sectional area A3 is smaller than the first, second and fourth cross-sectional areas A1, A2 and A4, such that the first portion 141′, second portion 141″ and connecting portion 142 are prevented from travelling through the hole in the tissue portion 610 in a direction perpendicular to the first, second and third planes P1, P2 and P3.

Hereby, the second portion 141″ and the connecting portion 142 can be held in position by the tissue portion 610 of the patient also when the first portion 141′ is disconnected from the connecting portion 142.

It is to be understood that the illustrated planes P1, P2, P3 and P4 are merely an example of how such planes may intersect the device 100. Other arrangements of planes are possible, as long as the conditions above are fulfilled, i.e. that the portions have cross-sectional areas, wherein the third cross-sectional area in the third plane P3 is smaller than the first, second and fourth cross-sectional areas, and that the planes P1, P2, P3 and P4 are parallel to each other.

The connecting portion 142 illustrated in FIG. 126 may be defined as a connecting portion 142 comprising a flange 626. The flange 626 thus comprises the fourth cross-sectional area A4 such that the flange 626 is prevented from travelling through the hole in the tissue portion 610 in a direction perpendicular to the first, second and third planes P1, P2 and P3. The flange 626 may protrude in a direction parallel to the first, second, third and fourth planes P1, P2, P3 and P4. This direction is perpendicular to a central extension C1 of the connecting portion 142.

The connecting portion 142 is not restricted to flanges, however. Other protruding elements may additionally or alternatively be incorporated into the connecting portion 142. As such, the connecting portion 142 may comprise at least one protruding element comprising the fourth cross-sectional area A4, such that the at least one protruding element is prevented from travelling through the hole in the tissue portion 610, such that the second portion 141″ and the connecting portion 142 can be held in position by the tissue portion 610 of the patient also when the first portion 141′ is disconnected from the connecting portion 142. The at least one protruding element may protrude in a direction parallel to the first, second, third and fourth planes P1, P2, P3 and P4. This direction is perpendicular to a central extension C1 of the connecting portion 142. As such, the at least one protruding element will also comprise the third surface configured to engage the first tissue surface 616 of the first side 612 of the tissue portion 610.

The connecting portion 142 may comprise a hollow portion 628. The hollow portion 628 may provide a passage between the first and second portions 141′, 141″. In particular, the hollow portion 628 may house a conduit for transferring fluid from the first portion 141′ to the second portion 141″. The hollow portion 628 may also comprise or house one or several connections or electrical leads for transferring energy and/or communication signals between the first portion 141′ and the second portion 141″.

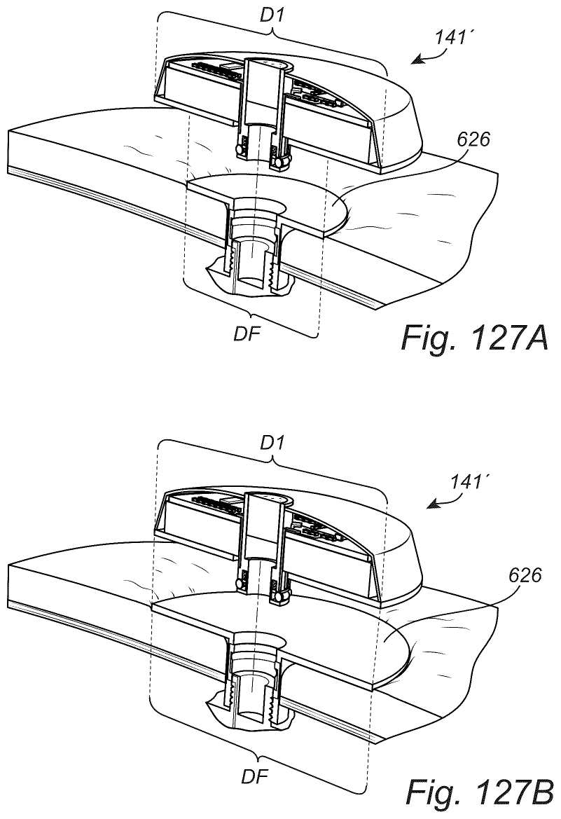

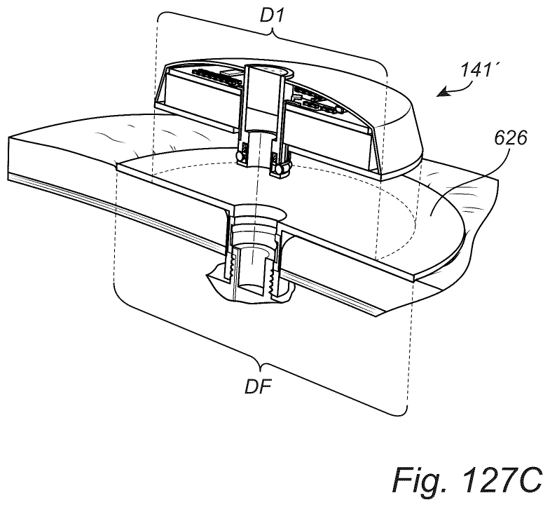

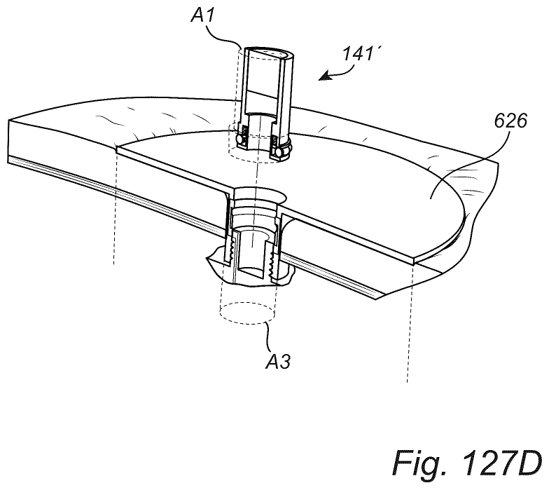

Some relative dimensions of the device 100 will now be described with reference to FIGS. 126 and 127A-127C, however it is to be understood that these dimensions may also apply to other embodiments of the device 100. The at least one protruding element 626 may have a height HF in a direction perpendicular to the fourth plane being less than a height H1 of the first portion 141′ in said direction. The height HF may alternatively be less than half of said height H1 of the first portion 141′ in said direction, less than a quarter of said height H1 of the first portion 141′ in said direction, or less than a tenth of said height H1 of the first portion 141′ in said direction.

The height H1 of the first portion 141′ in a direction perpendicular to the first plane may be less than a height H2 of the second portion 141″ in said direction, such as less than half of said height H2 of the second portion 141″ in said direction, less than a quarter of said height H2 of the second portion 141′″ in said direction, or less than a tenth of said height H2 of the second portion 141″ in said direction.

The at least one protruding element 626 may have a diameter DF in the fourth plane being one of less than a diameter D1 of the first portion 141′ in the first plane, equal to a diameter D1 of the first portion 141′ in the first plane, and larger than a diameter D1 of the first portion 141′ in the first plane.

Similarly, the cross-sectional area of the at least one protruding element 626 in the fourth plane may be less, equal to, or larger than a cross-sectional area of the first portion in the first plane.

The at least one protruding element 626 may have a height HF in a direction perpendicular to the fourth plane being less than a height HC of the connecting portion 142 in said direction. Here, the height HC of the connecting portion 142 is defined as the height excluding the at least one protruding element, which forms part of the connecting portion 142. The height HF may alternatively be less than half of said height HC of the connecting portion 142 in said direction, less than a quarter of said height HC of the connecting portion 142 in said direction, or less than a tenth of said height HC of connecting portion 142 in said direction.

As shown in FIG. 127D, the first portion 141′ may have a first cross-sectional area A1 being equal to or smaller than the third cross-sectional area A3 of the connecting portion 142. In particular, the first portion 141′ does not necessarily need to provide a cross-sectional area being larger than the third cross-sectional area of connecting portion 142, intended to pass through a hole in the tissue, if the connecting portion 142 provides an additional cross-sectional area being larger than the third cross-sectional area of the connecting portion 142. The first portion 141′ as illustrated in FIG. 127D may comprise the components discussed elsewhere in the present disclosure, although not shown, such as an energy storage unit, receiver, transmitter, etc.

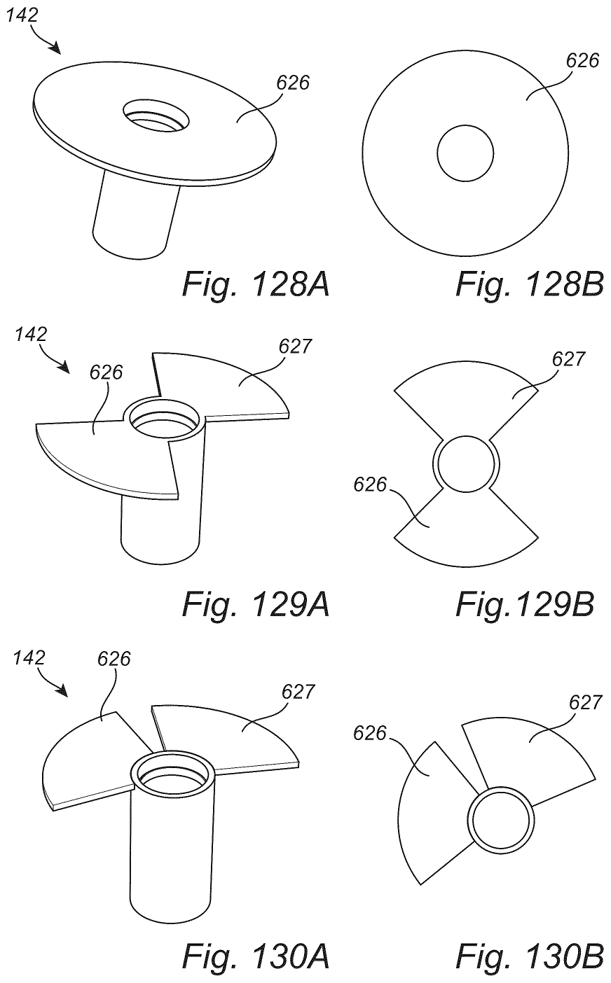

As shown in FIGS. 128A-128B, the at least one protruding element 626 may have an annular shape, such as a disk shape. However, elliptical, elongated and/or other polyhedral or irregular shapes are also possible. In the illustrated embodiment, the at least one protruding element 626 extends a full revolution around the center axis of the connecting portion 142. However, other arrangements are possible, wherein the at least one protruding element 626 constitute a partial circle sector. In the case of a plurality of protruding elements, such plurality of protruding elements may constitute several partial circle sectors.

As shown in FIGS. 129A-129B, 130A-130B, the connecting portion 142 may comprise at least two protruding elements 626, 627. For example, the connecting portion 142 may comprise at least three, four, five, fix, seven, eight, nine, ten protruding elements, and so on. In such embodiments, the at least two protruding elements 626, 627 may together comprise the fourth cross-sectional area, thus providing a necessary cross-sectional area to prevent the first portion and second portion from travelling through the hole in the tissue portion.

The at least two protruding elements 626, 627 may be symmetrically arranged about the central axis of the connecting portion, as shown in FIG. 129A-129B, or asymmetrically arranged about the central axis of the connecting portion, as shown in FIGS. 130A. 130B. In particular, the at least two protruding elements 626, 627 may be asymmetrically arranged so as to be located towards one side of the connecting portion 142, as shown in FIGS. 130A-130B. The arrangement of protruding element(s) may allow the device 100, and in particular the connecting portion 142, to be placed in areas of the patient where space is limited in one or more directions.

The first portion 141′ may comprise a first energy storage unit for supplying the device 100 with energy.

Although one type or embodiment of the implantable energized medical device 100, which may be referred to as a remote unit in other parts of the present disclosure, may fit most patients, it may be necessary to provide a selection of implantable energized medical devices 100 or portions to be assembled into implantable energized medical devices 100. For example, some patients may require different lengths, shapes, sizes, widths or heights depending on individual anatomy. Furthermore, some parts or portions of the implantable energized medical device 100 may be common among several different types or embodiments of implantable energized medical devices, while other parts or portions may be replaceable or interchangeable. Such parts or portions may include energy storage devices, communication devices, fluid connections, mechanical connections, electrical connections, and so on.

To provide flexibility and increase user friendliness, a kit of parts may be provided. The kit preferably comprises a group of one or more first portions, a group of one or more second portions, and a group of one or more connecting portions, the first portions, second portions and connecting portions being embodied as described throughout the present disclosure. At least one of the groups comprises at least two different types of said respective portions. By the term “type”, it is hereby meant a variety, class or embodiment of said respective portion.

In some embodiments of the kit, the group of one or more first portions, the group of one or more second portions, and the group of one or more connecting portions, comprise separate parts which may be assembled into a complete implantable energized medical device. The implantable energized medical device may thus be said to be modular, in that the first portion, the second portion, and/or the connecting portion may be interchanged for another type of the respective portion.

In some embodiments, the connecting portion form part of the first portion or the second portion.

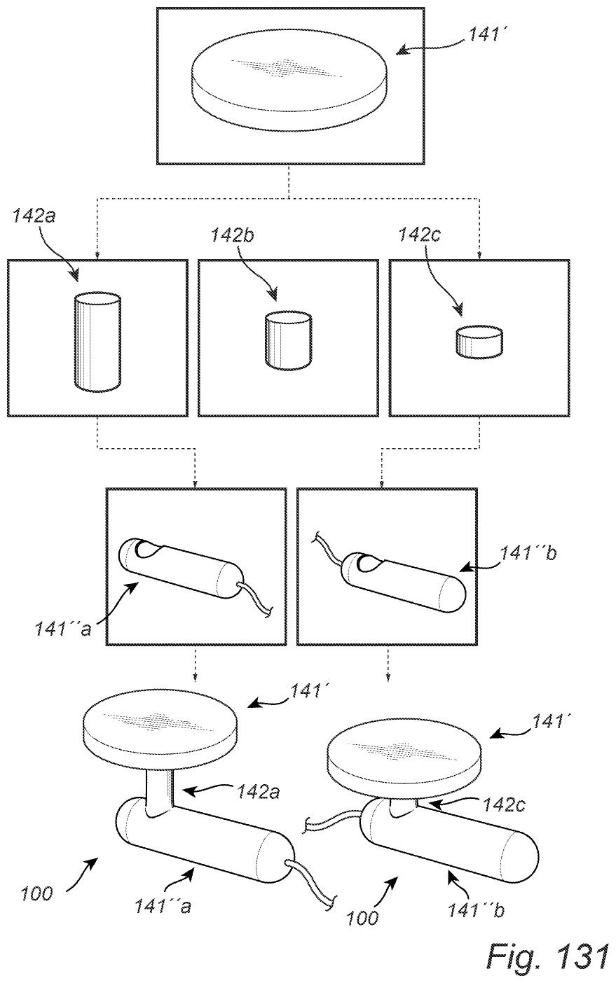

With reference to FIG. 131, the kit for assembling the implantable energized medical device comprises a group 650 of one or more first portions 141′, in the illustrated example a group of one first portion 141′, a group 652 of one or more connecting portions 142, in the illustrated example a group of three connecting portions 142, and a group 654 of one or more second portions 141″, in the illustrated example a group of two second portions 141″. For simplicity, all types and combinations of first portions, second portions and connecting portions will not be illustrated or described in detail.

Accordingly, the group 652 of one or more connecting portions 142 comprise three different types of connecting portions 142. Here, the different types of connecting portions 142 comprise connecting portions 142a, 142b, 142c having different heights. Furthermore, the group 654 of one or more second portions 141″ comprise two different types of second portions 141″.

Here, the different types of second portions 141″ comprise a second portion 141″ a being configured to excentrically connect to a connecting portion, having a first end and a second end as described in other parts of the present disclosure, wherein the second end of the second portion 141″ a comprises or is configured for at least one connection for connecting to an implant being located in a caudal direction from a location of the implantable energized medical device in the patient, when the device is assembled. In the illustrated figure, the at least one connection is visualized as a lead or wire.

However, other embodiments are possible, including the second end comprising a port, connector or other type of connective element for transmission of power, fluid, and/or signals.

Furthermore, the different types of second portions 141″ comprise a second portion 141″b being configured to excentrically connect to a connecting portion, having a first end and a second end as described in other parts of the present disclosure, wherein the first end of the second portion 141″b comprises or is configured for at least one connection for connecting to an implant being located in a cranial direction from a location of the implantable energized medical device in the patient, when the device is assembled. In the illustrated figure, the at least one connection is visualized as a lead or wire. However, other embodiments are possible, including the first end comprising a port, connector or other type of connective element for transmission of power, fluid, and/or signals.

Thus, the implantable energized medical device may be modular, and different types of devices can be achieved by selecting and combining a first portion 141′, a connecting portion 142, and a second portion 141″, from each of the groups 652, 654, 656.

In the illustrated example, a first implantable energized medical device 100a is achieved by a selection of the first portion 141′, the connecting portion 142a, and the second portion 141″ a. Such device 100a may be particularly advantageous in that the connecting portion 142a may be able to extend through a thick layer of tissue to connect the first portion 141′ and the second portion 141′″ a. Another implantable energized medical device 100b is achieved by a selection of the first portion 141′, the connecting portion 142c, and the second portion 141″b. Such device may be particularly advantageous in that the connecting portion 142c has a smaller footprint than the connecting portion 142a, i.e. occupying less space in the patient. Owing to the modular property of the devices 100a and 100b, a practician or surgeon may select a suitable connecting portion as needed upon having assessed the anatomy of a patient. Furthermore, since devices 100a and 100b share a common type of first portions 141′, it will not be necessary for a practician or surgeon to maintain a stock of different first portions (or a stock of complete, assembled devices) merely for the sake of achieving a device having different connections located in the first end or second end of the second portion respectively, as in the case of second portions 141″ a, 141″b.

The example illustrated in FIG. 131 is merely exemplifying to display the idea of a modular implantable energized medical device 100. The group 650 of one or more first portions 141′ may comprise a variety of different features, such as first portions with or without a first energy storage unit, with or without a first wireless energy receiver unit for receiving energy transmitted wirelessly by an external wireless energy transmitter, with or without an internal wireless energy transmitter, and/or other features as described throughout the present disclosure. Other features include different height, width, or length of the first portion. It is to be understood that first portions having one or more such features may be combined with a particular shape or dimensions to achieve a variety of first portions. The same applies to connecting portions and second portions.

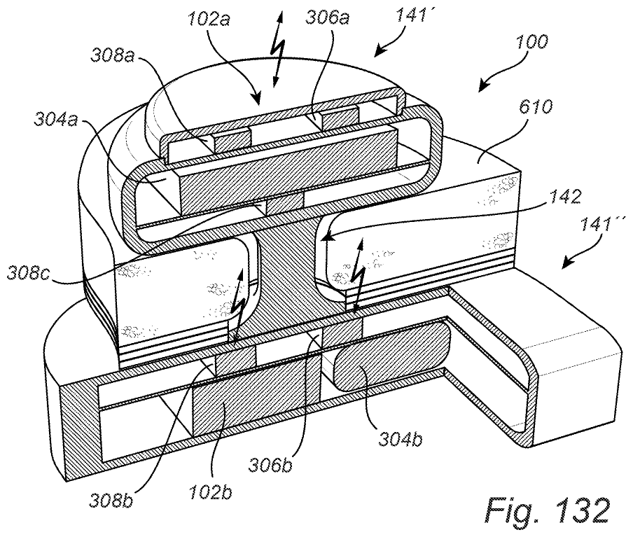

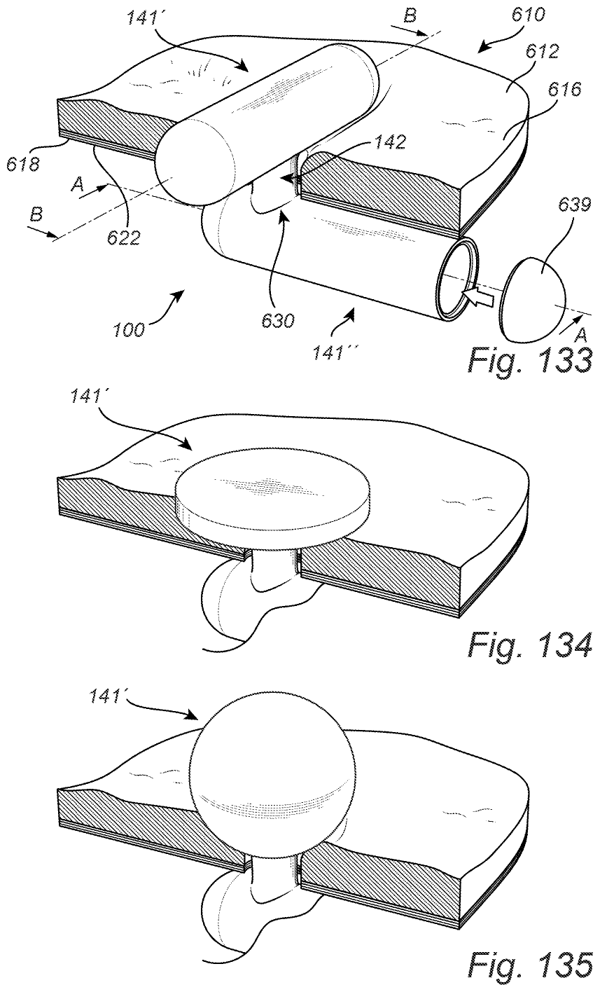

With reference to FIG. 132, an embodiment of an implantable energized medical device 100, which may be referred to as a remote unit in other parts of the present disclosure, will be described. The device 100 is configured to be held in position by a tissue portion 610 of a patient. The device 100 comprises a first portion 141′ configured to be placed on a first side of the tissue portion 610, the first portion 141′ having a first cross-sectional area in a first plane and comprising a first surface configured to face and/or engage a first tissue surface of the first side of the tissue portion 610. The device 100 further comprises a second portion 141″ configured to be placed on a second side of the tissue portion 610, the second side opposing the first side, the second portion 141″ having a second cross-sectional area in a second plane and comprising a second surface configured to engage a second tissue surface of the second side of the tissue portion 610. The device 100 further comprises a connecting portion 142 configured to be placed through a hole in the tissue portion 610 extending between the first and second sides of the tissue portion 610. The connecting portion 142 here has a third cross-sectional area in a third plane. The connecting portion 142 is configured to connect the first portion 141′ to the second portion 141″. Here, the first portion 141′ comprises a first wireless energy receiver 308a for receiving energy transmitted wirelessly by an external wireless energy transmitter, and an internal wireless energy transmitter 308a configured to transmit energy wirelessly to the second portion. Furthermore, the second portion here comprises a second wireless energy receiver 308b configured to receive energy transmitted wirelessly by the internal wireless energy transmitter 308a.

Although receivers and transmitters may be discussed and illustrated separately in the present disclosure, it is to be understood that the receivers and/or transmitters may be comprised in a transceiver. Furthermore, the receivers and/or transmitters in the first portion 141′ and second portion 141″ respectively may form part of a single receiving or transmitting unit configured for receiving or transmitting energy and/or communication signals, including data. Furthermore, the internal wireless energy transmitter and/or a first wireless communication receiver/transmitter may be a separate unit 308c located in a lower portion of the first portion 141′, referred to as a proximal end of the first portion 141′ in other parts of the present disclosure, close to the connecting portion 142 and the second portion 141″. Such placement may provide for that energy and/or communication signals transmitted by the unit 308c will not be attenuated by internal components of the first portion 141′ when being transmitted to the second portion 141″. Such internal components may include a first energy storage unit 304a.

The first portion 141′ here comprises a first energy storage unit 304a connected to the first wireless energy receiver 308a. The second portion comprises a second energy storage unit 304b connected to the second wireless energy receiver 308b. Such an energy storage unit may be a solid-state battery, such as a thionyl-chloride battery.

In some embodiments, the first wireless energy receiver 308a is configured to receive energy transmitted wirelessly by the external wireless energy transmitter and store the received energy in the first energy storage unit 304a. Furthermore, the internal wireless energy transmitter 308a is configured to wirelessly transmit energy stored in the first energy storage unit 304a to the second wireless energy receiver 308b, and the second wireless energy receiver 308b is configured to receive energy transmitted wirelessly by the internal wireless energy transmitter 308a and store the received energy in the second energy storage unit 305b.

The first energy storage unit 304a may be configured to store less energy than the second energy storage unit 304b, and/or configured to be charged faster than the second energy storage unit 304b.

Hereby, charging of the first energy storage unit 304a may be relatively quick, whereas transfer of energy from the first energy storage unit 304a to the second energy storage unit 304b may be relatively slow. Thus, a user can quickly charge the first energy storage unit 304a and will not during such charging be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b, via the first wireless energy transmitter 308a, c and the second wireless energy receiver 308b.

The first portion may comprise a first controller comprising at least one processing unit 306a. The second portion may comprise a second controller comprising at least one processing unit 306b. At least one of the first and second processing unit 306a, 306b may be connected to a wireless transceiver 308a, b, c for communicating wirelessly with an external device.

The first controller may be connected to a first wireless communication receiver 308a, c in the first portion 141′ for receiving wireless communication from an external device and/or from a wireless communication transmitter 308b in the second portion 141″. Furthermore, the first controller may be connected to a first wireless communication transmitter 308a, c in the first portion 141′ for transmitting wireless communication to a second wireless communication receiver 308b in the second portion 141″.

The second controller may be connected to the second wireless communication receiver 308b for receiving wireless communication from the first portion 141′. The second controller may further be connected to a second wireless communication transmitter 308b for transmitting wireless communication to the first portion 141′.

In some embodiments, the first wireless energy receiver 308a comprises a first coil, and the wireless energy transmitter 308a, c comprises a second coil, as shown in FIG. 137aa.

The device may further comprise at least one sensor (not shown) for providing input to at least one of the first and second controller. Such sensor data may be transmitted to an external device via the first wireless communication transmitter 308a and/or the second wireless communication transmitter 308b. The sensor may be or comprise a sensor configured to sense a physical parameter of the device 100. The sensor may also be or comprise a sensor configured to sense at least one of a temperature of the device 100, a temperature of a body engaging portion, a parameter related to the power consumption of the device, a parameter related to the power consumption of a body engaging portion, a parameter related to a status of at least one of the first and second energy storage units 304a, 304b, such as a health status of at least one of the first and second energy storage units 304a, 304b, a parameter related to the wireless transfer of energy from a source external to the body of the patient, and a hydraulic pressure. By the term “health status” it is hereby meant a status indicating the current total capacity of the energy storage unit as compared to the total capacity of an unused energy storage unit. The sensor may also be or comprise a sensor configured to sense a physiological parameter of the patient, such as at least one of a parameter related to the patient swallowing, a local temperature, a systemic temperature, a blood saturation, a blood oxygenation, a blood pressure, a parameter related to an ischemia marker, or pH. The sensor configured to sense a parameter related to the patient swallowing may comprise at least one of a motility sensor, a sonic sensor, an optical sensor, and a strain sensor. The sensor configured to sense pH may be configured to sense the acidity in the stomach.

The sensor may be configured to sense a temperature of the device 100, to avoid excessive heating of tissue connected to the device during operation of the device, or during operation of an external implant using the device, or charging of an energy storage unit in the device 100. Excessive heating may also damage the device and/or the energy storage unit. Excessive heating may also be an indicator that something is wrong with the device and may be used for triggering an alarm function for alerting the patient or physician. The sensor may also be configured to sense a parameter related to the power consumption of the device 100 or the power consumption of an external implant being powered by the device 100, to avoid excessive power consumption which may drain and/or damage the energy storage unit of the device 100. Excessive power consumption may also be an indicator that something is wrong with the device 100 and may be used for triggering an alarm function for alerting the patient or physician.

Wireless energy receivers and/or communication receivers and/or transmitters in the first portion 141′ may be configured to receive energy from and/or communicate wirelessly with an external device outside the body using electromagnetic waves at a frequency below 100 kHz, or more specifically below 40 kHz, or more specifically below 20 kHz. The wireless energy receivers and/or communication receivers and/or transmitters in the first portion 141′ may thus be configured to communicate with the external device using “Very Low Frequency” communication (VLF). VLF signals have the ability to penetrate a titanium housing of the implantable energized medical device, such that the electronics of the implantable medical device can be completely encapsulated in a titanium housing. In addition, or alternatively, communication and energy transfer between the first portion 141′ and second portion 141″ may be made using VLF signals. In such embodiments, receivers and transmitters (for energy and/or communication) of the first portion 141′ and second portion 141″ are configured accordingly.

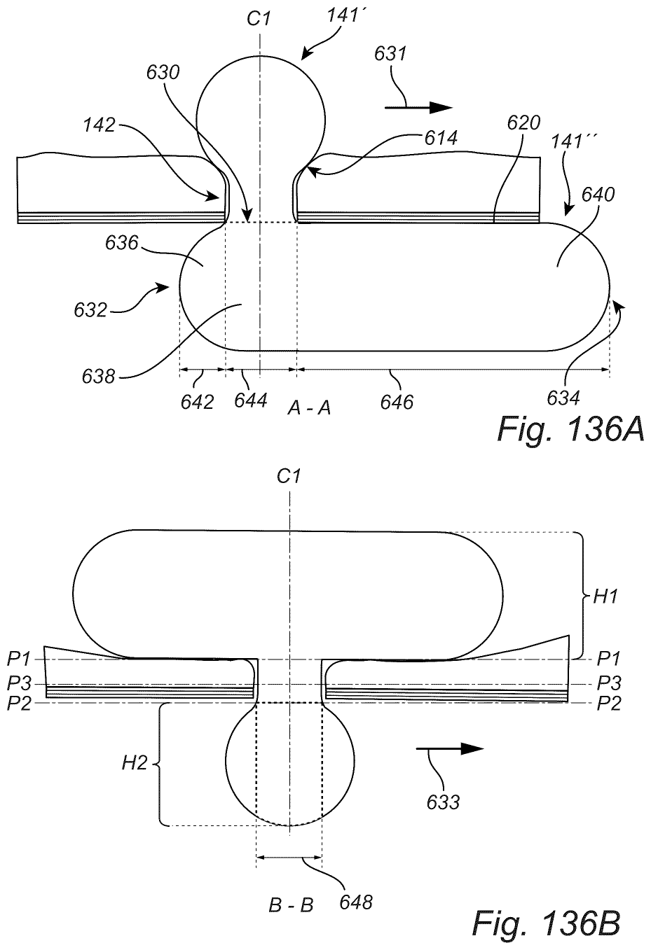

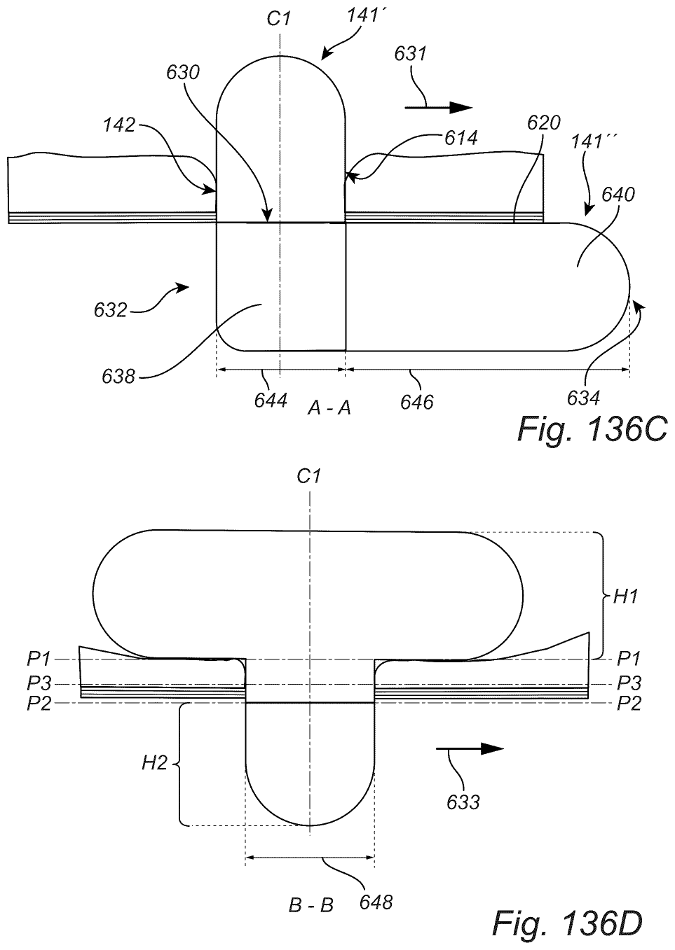

With reference to FIGS. 133, 136A and 136B, an embodiment of an implantable energized medical device 100, which may be referred to as a remote unit in other parts of the present disclosure, will be described. The device 100 is configured to be held in position by a tissue portion 610 of a patient.

The device 100 comprises a first portion 141′ configured to be placed on a first side 612 of the tissue portion 610, the first portion 141′ having a first cross-sectional area A1 in a first plane P1 and comprising a first surface 614 configured to face and/or engage a first tissue surface 616 of the first side 612 of the tissue portion 610. The device 100 further comprises a second portion 141″ configured to be placed on a second side 618 of the tissue portion 610, the second side 618 opposing the first side 612, the second portion 141″ having a second cross-sectional area A2 in a second plane P2 and comprising a second surface 620 configured to engage a second tissue surface 622 of the second side 618 of the tissue portion 610. The device 100 further comprises a connecting portion 142 configured to be placed through a hole in the tissue portion 610 extending between the first and second sides 612, 618 of the tissue portion 610. The connecting portion 142 here has a third cross-sectional area A3 in a third plane P3. The connecting portion 142 is configured to connect the first portion 141′ to the second portion 141″. In the illustrated embodiment, a connecting interface 630 between the connecting portion 142 and the second portion 141″ is excentric with respect to the second portion 141″.

The first portion 141′ has an elongated shape in the illustrated embodiment of FIG. 125. Similarly, the second portion 141″ has an elongated shape. However, the first portion 141′ and/or second portion 141″ may assume other shapes, such as a flat disk e.g. having a width and length being larger than the height, a sphere, an ellipsoid, or any other polyhedral or irregular shape, some of these being exemplified in FIGS. 133-135.

As illustrated in FIGS. 136A and 136B, the connecting interface 630 between the connecting portion 142 and the second portion 141″ may be excentric, with respect to the second portion 141″ in a first direction 631, but not in a second direction 633 being perpendicular to the first direction. The first direction 631 is here parallel to the line A-A, to the second plane P2, and to a length of the second portion 141″. The second direction 633 is here parallel to the line B-B, to the second plane P2, and to a width of the second portion 141″. It is also possible that the connecting interface between the connecting portion 142 and the second portion 141″ is excentric, with respect to the second portion 141″, in the first direction 631 as well as in the second direction 633 being perpendicular to the first direction 631.

Similarly, a connecting interface between the connecting portion 142 and the first portion 141′ may be excentric with respect to the first portion 141′ in the first direction 631, and/or in the second direction 633.

The first portion 141′, connecting portion 142 and second portion 141″ may structurally form one integral unit. It is however also possible that the first portion 141′ and the connecting portion 142 structurally form one integral unit, while the second portion 141″ form a separate unit, or, that the second portion 141″ and the connecting portion 142 structurally form one integral unit, while the first portion 141′ form a separate unit.

Additionally, or alternatively, the second portion 141″ may comprise a removable and/or interchangeable portion 639. In some embodiments, the removable portion 639 may form part of a distal region which will be further described in other parts of the present disclosure. A removable portion may also form part of a proximal region. Thus, the second portion 141″ may comprise at least two removable portions, each being arranged at a respective end of the second portion 141″. The removable portion 639 may house, hold or comprise one or several functional parts of the device 100, such as gears, motors, connections, reservoirs, and the like as described in other parts of the present disclosure. An embodiment having such removable portion 639 will be able to be modified as necessary to circumstances of a particular patient.

In the case of the first portion 141′, connecting portion 142 and second portion 141″ structurally forming one integral unit, the excentric connecting interface between the connecting portion 142 and the second portion 141″, with respect to the second portion 141″, will provide for that the device 100 will be able to be inserted into the hole in the tissue portion. The device 100 may for example be inserted into the hole at an angle, similar to how a foot is inserted into a shoe, to allow most or all of the second portion 141″ to pass through the hole, before it is angled, rotated, and/or pivoted to allow any remaining portion of the second portion 141″ to pass through the hole and allow the device 100 to assume its intended position.

As illustrated in FIGS. 133, 134 and 135, the first portion 141′ may assume a variety of shapes, such as an oblong shape, a flat disk shape, a spherical shape, or any other polyhedral or irregular shape. Similarly, the second portion 141″ may assume a variety of shapes, such as an oblong shape, a flat disk shape, a spherical shape, or any other polyhedral or irregular shape. The proposed shapes of the first and second portions 141′, 141″ may be mixed and combined to form embodiments not exemplified in the illustrated embodiments. For example, one or both of the first and second portions 141′, 141″ may have a flat oblong shape. In this context, the term “flat” is related to the height of the first or second portion 141′, 141″, i.e. in a direction parallel to a central extension C1 of the connecting portion 142. The term “oblong” is related to a length of the first or second portion 141′, 141″. A definition of such length is further discussed in other parts of the present disclosure.

With reference to FIGS. 136A-136B, the second portion 141″ has a first end 632 and a second end 634 opposing the first end 632. The length of the second portion 141″ is defined as the length between the first end 632 and the second end 634. The length of the second portion 141″ is furthermore extending in a direction being different to the central extension C1 of the connecting portion 142. The first end 632 and second end 634 are separated in a direction parallel to the second plane P2. Similarly, the first portion 141′ has a length between a first and a second end, the length extending in a direction being different to the central extension C1 of the connecting portion 142.

The second portion 141″ may be curved along its length. For example, one or both ends of the second portion 141″ may point in a direction being substantially different from the second plane P2, i.e. curving away from or towards the tissue portion when implanted. In some embodiments, the second portion 141″ curves within the second plane P2, exclusively or in combination with curving in other planes. The second portion 141″ may also be curved in more than one direction, i.e. along its length and along its width, the width extending in a direction perpendicular to the length.

The first and second ends 632, 634 of the second portion 141″ may comprise an elliptical point respectively. For example, the first and second ends 632, 634 may comprise a hemispherical end cap respectively. It is to be understood that also the first and second ends of the first portion 141′ may have such features.

The second portion 141″ may have at least one circular cross-section along the length between the first end 632 and second end 634, as illustrated in FIG. 133. It is however possible for the second portion 141″ to have at least one oval cross-section or at least one elliptical cross-section along the length between the first end 632 and the second end 634. Such cross-sectional shapes may also exist between ends in a width direction of the second portion 141″. Similarly, such cross-sectional shapes may also exist between ends in a length and/or width direction in the first portion 141′.

In the following paragraphs, some features and properties of the second portion 141″ will be described. It is however to be understood that these features and properties may also apply to the first portion 141′.

The second portion 141″ has a proximal region 636, an intermediate region 638, and a distal region 640. The proximal region 636 extends from the first end 632 to an interface between the connecting portion 142 and the second portion 141″, the intermediate region 638 is defined by the connecting interface 630 between the connecting portion 142 and the second portion 141″, and the distal region 640 extends from the connecting interface 630 between the connecting portion 142 and the second portion 141″ to the second end 634. The proximal region 636 is shorter than the distal region 640 with respect to the length of the second portion, i.e. with respect to the length direction 631. Thus, a heel (the proximal region) and a toe (the distal region) is present in the second portion 141″.

The second surface 620, configured to engage with the second tissue surface 622 of the second side 618 of the tissue portion 610, is part of the proximal region 636 and the distal region 640. If a length of the second portion 141″ is defined as x, and the width of the second portion 141″ is defined as y along respective length and width directions 631, 633 being perpendicular to each other and substantially parallel to the second plane P2, the connecting interface between the connecting portion 142 and the second portion 141″ is contained within a region extending from x>0 to x<x/2 and/or y>0 to y<y/2, x and y and 0 being respective end points of the second portion 141″ along said length and width directions. In other words, the connecting interface between the connecting portion 142 and the second portion 141″ is excentric in at least one direction with respect to the second portion 141″, such that a heel and a toe is formed in the second portion 141″.

The first surface 614 configured to face and/or engage the first tissue surface 616 of the first side 612 of the tissue portion 610 may be substantially flat. In other words, the first portion 141′ may comprise a substantially flat side facing towards the tissue portion 610. Furthermore, an opposing surface of the first portion 141′, facing away from the tissue portion 610, may be substantially flat. Similarly, the second surface 620 configured to engage the second tissue surface 622 of the second side 618 of the tissue portion 610 may be substantially flat. In other words, the second portion 141″ may comprise a substantially flat side facing towards the tissue portion 610. Furthermore, an opposing surface of the second portion 141″, facing away from the tissue portion 610, may be substantially flat.

The second portion 141″ may be tapered from the first end 632 to the second end 634, thus giving the second portion 141″ different heights and/or widths along the length of the second portion 141″. The second portion may also be tapered from each of the first end 632 and second end 634 towards the intermediate region 638 of the second portion 141″.

Some dimensions of the first portion 141′, the second portion 141″ and the connecting portion 142 will now be disclosed. Any of the following disclosures of numerical intervals may include or exclude the end points of said intervals.

The first portion 141′ may have a maximum dimension being in the range of 10 to 60 mm, such as in the range of 10 to 40 mm such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm. By the term “maximum dimension” it is hereby meant the largest dimension in any direction.

The first portion 141′ may have a diameter being in the range of 10 to 60 mm, such as in the range of 10 to 40 mm such as in the range of 10 to 30 mm, such as in the range of 10 to 25 mm, such as in the range of 15 to 40 mm, such as in the range of 15 to 35 mm, such as in the range of 15 to 30 mm, such as in the range of 15 to 25 mm.

The connecting portion 142 may have a maximum dimension in the third plane P3 in the range of 2 to 20 mm, such as in the range of 2 to 15 mm, such as in the range of 2 to 10 mm, such as in the range of 5 to 10 mm, such as in the range of 8 to 20 mm, such as in the range of 8 to 15 mm, such as in the range of 8 to 10 mm.

The second portion 141″ may have a maximum dimension being in the range of 30 to 90 mm, such as in the range of 30 to 70 mm, such as in the range of 30 to 60 mm, such as in the range of 30 to 40 mm, such as in the range of 35 to 90 mm, such as in the range of 35 to 70 mm, such as in the range of 35 to 60 mm, such as in the range of 35 to 40 mm.

The first portion has a first height H1, and the second portion has a second height H2, both heights being in a direction perpendicular to the first and second planes P1, P2. The first height may be smaller than the second height. However, in the embodiments illustrated in FIGS. 136A-136B, the first height H1 is substantially equal to the second height H2. Other height ratios are possible, for example the first height H1 may be less than ⅔ of the second height H2, such as less than ½ of the second height H2, such as less than ⅓ of the second height H2, such as less than ¼ of the second height H2, such as less than ⅕ of the second height H2, such as less than 1/10 of the second height H2.

As illustrated in FIGS. 136A-136B, the proximal region 636 has a length 642 being shorter than a length 646 of the distal region 640. The intermediate region 638 has a length 644, and a width 648. In some embodiments, the length 644 of the intermediate region 638 is longer than the width 648. In other words, the connecting interface between the connecting portion 142 and the second portion 141″ may be elongated, having a longer dimension (in the exemplified case, the length) and a shorter dimension (in the exemplified case, the width). It is also possible that the length 644 of the intermediate region 638 is shorter than the width 648 of the intermediate region 638.

The length 646 of the distal region 640 is preferably longer than the length 644 of the intermediate region 638, however, an equally long distal region 640 and intermediate region 638, or a shorter distal region 640 than the intermediate region 638, is also possible. The length 642 of the proximal region 636 may be shorter than, equal to, or longer than the length 644 of the intermediate region 638.

The length 644 of the intermediate region 638 is preferably less than half of the length of the second portion 141″, i.e. less than half of the combined length of the proximal region 636, the intermediate region 638, and the distal region 630. In some embodiments, the length 644 of the intermediate region 638 is less than a third of the length of the second portion 141″, such as less than a fourth, less than a fifth, or less than a tenth of the length of the second portion 141″.

The connecting portion may have one of an oval cross-section, an elongated cross-section, and a circular cross-section, in a plane parallel to the third plane P3. In particular, the connecting portion may have several different cross-sectional shapes along its length in the central extension C1.

FIGS. 136C-136D illustrate an embodiment similar to the one described in conjunction with FIGS. 136A-136B. However, the embodiment of FIGS. 136C-136D lacks a proximal portion, i.e. the second portion 141″ does not comprise a “heel”. Furthermore, such embodiment may have a connecting portion 142 having a length and width, in directions 631 and 633 respectively, being equal to a height of the second portion in a direction parallel to the central extension C1, as illustrated. Thus, the connecting portion 142 and the second portion 141″ may be constituted by a substantially uniformly wide body.

In some embodiments the distal region 640 is configured to be directed downwards in a standing patient, i.e. in a caudal direction when the device 100 is implanted. As illustrated in FIGS. 137A-137D, different orientations of the second portion 141″ relative the first portion 141′ are possible. In some embodiments, a connection between either the first portion 141′ and the connecting portion 142, or between the second portion 141″ and the connecting portion 142, may allow for a plurality of different connecting orientations. For example, a connection mechanism between the first portion 141′ and the connecting portion 142 (or between the second portion 141″ and the connecting portion 142) may possess a 90 degree rotational symmetry to allow the second portion 141′ to be set in four different positions with respect to the first portion 141, each differing from the other by 90 degrees. Other degrees of rotational symmetry are of course possible, such as 30 degrees, 45 degrees, 60 degrees, 120 degrees, 180 degrees and so on. In other embodiments there are no connective mechanism between any of the first portion 141′, the connecting portion 142, and the second portion 141″ (i.e. the portions are made as one integral unit), and in such cases different variants of the device 100 can be achieved during manufacturing. In other embodiments, the connective mechanism between the first portion 141′ and the connecting portion 142 (or between the second portion 141″ and the connecting portion 142) is non-reversible, i.e. the first portion 141′ and the second portion 141″ may initially be handled as separate parts, but the orientation of the second portion 141″ relative the first portion 141′ cannot be changed once it has been selected and the parts have been connected via the connecting portion 142.

The different orientations of the second portion 141″ relative the first portion 141′ may be defined as the length direction of the second portion 141″ having a relation or angle with respect to a length direction of the first portion 141′. Such angle may be 15 degrees, 30, 45, 60, 75 90, 105, 120, 135, 150, 165, 180, 195, 210, 225, 240, 255, 270, 285, 300, 315, 330, 345 or 360 degrees. In particular, the angle between the first portion 141′ and the second portion 141″ may be defined as an angle in the planes P1 and P2, or as an angle in a plane parallel to the tissue portion 610, when the device 100 is implanted. In the embodiment illustrated in FIGS. 137A-137D, the length direction of the second portion 141″ is angled by 0, 90, 180, and 270 degrees with respect to the length direction of the first portion 141′.

The second end 634 of the second portion 141″ may comprise one or several connections for connecting to an implant being located in a caudal direction from a location of the implantable energized medical device in the patient. Hereby, when the device 100 is implanted in a patient, preferably with the distal region 640 and second end 634 pointing downwards in a standing patient, the connections will be closer to the implant as the second end 634 will be pointing in the caudal direction whereas the first end 632 will be pointing in the cranial direction. It is also possible that the second end 634 of the second portion 141″ is configured for connecting to an implant, i.e. the second end 634 may comprise a port, connector or other type of connective element for transmission of power, fluid, and/or signals.

Likewise, the first end 632 of the second portion 141″ may comprise one or several connections for connecting to an implant being located in a cranial direction from a location of the implantable energized medical device in the patient. Hereby, when the device 100 is implanted in a patient, preferably with the distal region 640 and second end 634 pointing downwards in a standing patient, the connections will be closer to the implant as the first end 632 will be pointing in the cranial direction whereas the second end 634 will be pointing in the caudal direction. It is also possible that the first end 632 of the second portion 141″ is configured for connecting to an implant, i.e. the first end 632 may comprise a port, connector or other type of connective element for transmission of power, fluid, and/or signals.

Referring now to FIGS. 137e-k, 137m, 137n, 137p and 137q. The following will discuss some features of the first portion 141′, and in some cases additionally or alternatively of the connecting portion 142, which enable the first portion 141′ to increase its cross-sectional area in the first plane (i.e. to increase an area of the first surface configured to face the first tissue surface), and/or which enable the first portion 141′ to be rotated, translated, or otherwise moved in relation to the connecting portion 142. In some embodiments, the first portion 141′ will be configured to extend further away from the connecting portion 142 in or within the first plane. It is to be understood that these features can be combined with other features of the implantable energized medical device. In particular, the specific shape of the first portion, connecting portion and/or second portion in the illustrated embodiments are merely exemplary. Other shapes are possible, as discussed in the present disclosure. Accordingly, the elongated second portion 141″ does not necessarily need to be elongated as shown for example in FIG. 137e, and furthermore, the first portion 141′ does not necessarily need to have a semicircular shape.

With reference to FIG. 137e, an implantable energized medical device 100 is shown, wherein the first portion 141′ is configured and shaped such that an edge 710 of the first portion 141′ is substantially aligned with the connecting portion 142 with regard to the first direction 631. In other words, no part of the first portion 141′ protrudes forward of the connecting portion 142 with regard to the first direction 631. Hereby, insertion of the implantable energized medical device 100 may be facilitated, in particular when angled downwards, since the first portion 141′ will not abut the tissue until most or all of the second portion 141″ has been inserted through the hole in the tissue. Although the edge 710, as well as other edges of the first portion 141′, are hereby shown as having no radius, radiused edges are possible.

Thus, the edge 710 may have a radius, and/or the first portion 141′, and/or the second portion 141″ and/or the connecting portion 142, may comprise radiused edges.

With reference to FIGS. 137f and 137g, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714 being hingedly interconnected to allow the first element 712 to assume a first state (not shown) wherein the first element 712 is arranged on top of the second element 714, and a second state wherein the first element 712 is folded to be located adjacent or next to the second element 714. A similar configuration may be achieved by other means of interconnection between the first element 712 and second element 714, i.e. the configuration is not limited to a hinge-type connection. For example, the first element 712 and second element 714 may be constructed of a single piece of material being flexible enough to be able to fold over itself to assume the first and second state respectively.

Preferably, the first and second element 712, 714 are interconnected and formed such that a transition between the first and second element 712, 714 along the first direction 631 is flush.

Furthermore, while in the first state, the first portion 141′ may possess the same feature as discussed in conjunction with FIG. 137e, i.e. the first portion 141′ may be substantially aligned with the connecting portion 142.

With reference to FIGS. 137h and 137i, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714. The second element 714 here comprises a slot 715 configured to partially or fully house the first element 712. The first element 712 is configured to rotate about an axis to assume a first state, wherein the first element 712 is partially or completely housed in within the slot 715, and a second state wherein the first element 712 protrudes from the slot 715 to increase the first cross-sectional area. The first element 712 may be configured to rotate 180 degrees about the axis. In the illustrated example, the first and second elements 712, 714 are shaped as semi-circles and form a shape conforming to a full circle in the second state. However, it is also possible that the first element 712 only rotate about the axis up to 90 degrees, thus forming a shape conforming to three quarters of a circle in the second state. Other shapes are also possible, e.g. polygons.

With reference to FIGS. 137j and 137k, a similar configuration as described with reference to

FIGS. 137h and 137i is shown. However, here the second element 714 does not comprise a slot, and the first element is thus not housed in a slot. Instead, the first element 712 is arranged on top of the second element 714 (similar to the embodiment of FIGS. 137f and 137g). The first portion 141′ is here configured to have its surface area increased, in particular the first cross-sectional area is increased, thereby increasing an area of the first surface configured to face (and in some embodiments also configured to contact) the first tissue surface. The first element 712 is configured to rotate about an axis to assume a first state, wherein the first element 712 is partially or completely arranged on top of the second element 714. Here, “completely arranged on top of” means that the first element 712 is confined within the borders of the second element 714. By rotation of the first element 712 about the axis, the first element 712 can assume a second state wherein the first element 712 protrudes over an edge or border of the second element 714 to increase the first cross-sectional area. The first element 712 may be configured to rotate 180 degrees about the axis. However, it is also possible that the first element 712 only rotate about the axis up to 90 degrees. Other shapes of the first and second element 712, 714 are also possible, e.g. polygons.

With reference to FIGS. 137m and 137n, a first portion 141′ is shown being configured to have its surface area increased. Here, the first cross-sectional area is increased, thereby increasing an area of the first surface configured to face (and in some embodiments also configured to contact) the first tissue surface. In the illustrated embodiment, the first portion 141′ comprises a first element 712 and a second element 714. The first element 712 here comprises a slot configured to partially or completely house the second element 714. The first element 712 is configured to assume a first state, as shown in FIG. 137m, wherein the second element 714 is arranged partially or fully within the slot of the first element 712, and a second state, as shown in FIG. 137n, wherein the first element 712 has been moved in a first direction to cause the second element 714 to protrude from the slot of the first element 712, and to cause the first element 712 to extend further away from the connecting portion 142 in the first plane. As will be understood, other variations are possible, e.g. the second element 714 may comprise the slot, and the first element 712 may be partially or fully housed within such slot, and subsequently the first element 712 or the second element 714 may be moved to protrude from such slot.

With reference to FIGS. 137p and 137q, a first portion 141′ is shown being configured to be moved in relation to the connecting portion 142. The expression “configured to be moved” may in this context be interpreted as the first portion 141′ being configured to assume at least two different positions with regard to the connecting portion 142 while still remaining in direct contact with the connecting portion. Here, the connecting portion 142 comprises a protruding element 717 and the first portion 141′ comprises a slot 718, wherein the protruding element 717 is configured to slide within the slot 718 along a predetermined path, e.g. in a first direction and a direction opposite said first direction. The protruding element 717 may be configured to be interlocked within the slot 718 such that the protruding element 717 can only be removed from the slot 718 in a preconfigured position. In other embodiments, the protruding element 717 may be permanently enclosed within the slot 718. By sliding the first portion 141′ in the first direction, an extension of the first portion 141′ in the first plane with respect to the connecting portion 142 will be able to be adjusted. Any position between the endpoints of the slot 718 may be able to be assumed by the first portion 141′. In particular, first portion 141′ and/or the connecting portion 142 may comprise a locking mechanism configured to secure a position of the first portion 141′ in relation to the connecting portion 142. Such locking mechanism may rely on flexible parts being biased towards each other to maintain the first portion 141′ and connecting portion 142 in a fixed position in relation to each other. Other possible locking mechanisms include the use of friction, snap-locking means, etc.

With reference to FIGS. 137r and 137s, an embodiment of an implantable energized medical device 100, which may be referred to as a remote unit in other parts of the present disclosure, will be described. The device 100 is configured to be held in position by a tissue portion 610 of a patient. The device 100 comprises a first portion 141′ configured to be placed on a first side 612 of the tissue portion 610, the first portion 141′ having a first cross-sectional area in a first plane and comprising a first surface 614 configured to face and/or engage a first tissue surface 616 of the first side 612 of the tissue portion 610. The device 100 further comprises a second portion 141″ configured to be placed on a second side 618 of the tissue portion 610, the second side 618 opposing the first side 612, the second portion 141″ having a second cross-sectional area in a second plane and comprising a second surface 620 configured to engage a second tissue surface 622 of the second side 618 of the tissue portion 610. The device 100 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″.

With reference to FIG. 137t, the first cross-sectional area has a first cross-sectional distance CD1a and a second cross-sectional distance CD2a, the first and second cross-sectional distances CD1a, CD2a being perpendicular to each other and the first cross-sectional distance CD1a being longer than the second cross-sectional distance CD2a. Furthermore, the second cross-sectional area has a first cross-sectional distance CD1b and a second cross-sectional distance CD2b, the first and second cross-sectional distances CD2a, CD2b being perpendicular to each other and the first cross-sectional distance CD1b being longer than the second cross-sectional distance CD2b. The first cross-sectional distance CD1a of the first cross-sectional area and the first cross-sectional distance CD1b of the second cross-sectional area are rotationally displaced in relation to each other with an angle exceeding 45° to facilitate insertion of the second portion 141″ through the hole in the tissue portion. In the embodiment illustrated in FIG. 137t, the rotational displacement is 90°.

The rotational displacement of the first portion 141′ and the second portion 141″ forms a cross-like structure, being particularly advantageous in that insertion through the hole in the tissue portion 610 may be facilitated, and once positioned in the hole in the tissue portion 610 a secure position may be achieved. In particular, if the device 100 is positioned such that the second portion 141″ has its first cross-sectional distance CD1b extending along a length extension of the hole 611 in the tissue portion 610, insertion of the second portion 141″ through the hole 611 may be facilitated. Furthermore, if the first portion 141′ is then displaced in relation to the second portion 141″ such that the first cross-sectional distance CD1a of the first portion 141′ is displaced in relation to a length extension of the hole 611, the first portion 141′ may be prevented from travelling through the hole 611 in the tissue portion. In these cases, it is particularly advantageous if the hole 611 in the tissue portion is oblong, ellipsoidal, or at least has one dimension in one direction being longer than a dimension in another direction. Such oblong holes in a tissue portion may be formed for example in tissue having a fiber direction, where the longest dimension of the hole may be aligned with the fiber direction.

In the embodiment illustrated in FIG. 137r, the first surface 614 of the first portion 141′ is flat, thus providing a larger contact surface to the first tissue surface 616 and consequently less pressure on the tissue portion. A more stable position may also be achieved by the flat surface. Also the second surface 620 of the second portion 141″ may be flat. However, other shapes, such as those described in other parts of the present disclosure, are possible.

As shown in FIG. 137t, the connecting portion 142 may have an elongated cross-section in the third plane. It may be particularly advantageous if the connecting portion 142 has a longer length 644 than width 648, said length 644 extending in the same direction as a length direction of the second portion 141″, i.e. in the same direction as an elongation of the second portion 141″. Hereby, the elongation of the connecting portion 142 may run in the same direction as an elongation of the hole in the tissue portion.

With reference to FIG. 137u, the rotational displacement of first cross-sectional distance of the first cross-sectional area and the first cross-sectional distance of the second cross-sectional area is shown, here at an angle about 45°. Accordingly, there is a rotational displacement, in the first, second and third planes, between a length direction 633 of the first portion 141′ and a length direction 631 of the second portion 141″. Other angles of rotational displacement are possible, such as 60°, 75, 90°, 105°, 120°, 135°, etc.

One and the same device 100 may be capable of assuming several different arrangements with regards to rotational displacement of the first portion 141′ and the second portion 141″. In particular, this is possible when the first portion 141′ and/or the second portion 141″ is configured to detachably connect to the interconnecting portion 142. For example, a connection mechanism between the first portion 141′ and the connecting portion 142, or between the second portion 141″ and the connecting portion 142, may possess a rotational symmetry to allow the first portion 141′ to be set in different positions in relation to the connecting portion 142 and in extension also in relation to the second portion 141″. Likewise, such rotational symmetry may allow the second portion 142″ to be set in different positions in relation to the connecting portion 142 and in extension also in relation to the first portion 141′.

With reference to FIGS. 137x-137z, a procedure of insertion of the device 100 in a tissue portion 610 will be described. The device 100 may be oriented such that a length direction 631 of the second portion 141″ points downwards into the hole 611. Preferably, the second portion 141″ is positioned such that it is inserted close to an edge of the hole 611. The second portion 141″ may then be inserted partially through the hole 611, until the point where the first portion 141′ abuts the first tissue surface 616. Here, a 90° rotational displacement between the first portion 141′ and the second portion 141″, as described above, will allow a relatively large portion of the second portion 141″ to be inserted before the first portion 141′ abuts the first tissue surface 616. Subsequently, the device 100 may be pivoted to slide or insert the remaining portion of the second portion 141″ through the hole 611. While inserting the remaining portion of the second portion 141″, the tissue may naturally flex and move to give way for the second portion 141″. Upon having fully inserted the second portion 141″ through the hole 611, such that the second portion 141″ is completely located on the other side of the tissue portion 610, the tissue may naturally flex back.

With reference to FIG. 137aa, an embodiment of an implantable energized medical device 100, which may be referred to as a remote unit in other parts of the present disclosure, will be described. The device 100 is configured to be held in position by a tissue portion 610 of a patient. The device 100 comprises a first portion 141′ configured to be placed on a first side 612 of the tissue portion 610, the first portion 141′ having a first cross-sectional area in a first plane and comprising a first surface 614 configured to face and/or engage a first tissue surface of the first side 612 of the tissue portion 610. The device 100 further comprises a second portion 141″ configured to be placed on a second side 618 of the tissue portion 610, the second side 618 opposing the first side 612, the second portion 141″ having a second cross-sectional area in a second plane and comprising a second surface 620 configured to engage a second tissue surface of the second side 618 of the tissue portion 610. The device 100 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″.

At least one of the first portion and the second portion comprises at least one coil embedded in a ceramic material, the at least one coil being configured for at least one of: receiving energy transmitted wirelessly, transmitting energy wirelessly, receiving wireless communication, and transmitting wireless communication. In the illustrated embodiment, the first portion 141′ comprises a first coil 658 and a second coil 660, and the second portion 141″ comprises a third coil 662. The coils are embedded in a ceramic material 664.

As discussed in other part of the present disclosure, the first portion 141′ may comprise a first wireless energy receiver configured to receive energy transmitted wirelessly from an external wireless energy transmitter, and further the first portion 141′ may comprise a first wireless communication receiver. The first wireless energy receiver and the first wireless communication receiver may comprise the first coil. Accordingly, the first coil may be configured to receive energy wirelessly, and/or to receive communication wirelessly.

By the expression “the receiver/transmitter comprising the coil” it is to be understood that said coil may form part of the receiver/transmitter.

The first portion 141′ comprises a distal end 665 and a proximal end 666, here defined with respect to the connecting portion 142. In particular, the proximal end 666 is arranged closer to the connecting portion 142 and closer to the second portion 141″ when the device 100 is assembled. In the illustrated embodiment, the first coil 658 is arranged at the distal end 665.

The first portion 141′ may comprise an internal wireless energy transmitter, and further a first wireless communication transmitter. In some embodiments, the internal wireless energy transmitter and/or the first wireless communication transmitter comprises the first coil 658. However, in some embodiments the internal wireless energy transmitter and/or the first wireless communication transmitter comprises the second coil 660. The second coil 660 is here arranged at the proximal end 665 of the first portion 141′. Such placement of the second coil 660 may provide for that energy and/or communication signals transmitted by the second coil 660 will not be attenuated by internal components of the first portion 141′ when being transmitted to the second portion 141″.

In some embodiments, the first wireless energy receiver and the internal wireless energy transmitter comprises a single coil embedded in a ceramic material. Accordingly, a single coil may be configured for receiving energy wirelessly and for transmitting energy wirelessly. Similarly, the first wireless communication receiver and the first wireless communication transmitter may comprise a single coil embedded in a ceramic material. Even further, in some embodiments a single coil may be configured for receiving and transmitting energy wirelessly, and for receiving and transmitting communication signals wirelessly.

The coils discussed herein are preferably arranged in a plane extending substantially parallel to the tissue portion 610.

The second portion 141″ may comprise a second wireless energy receiver, and/or a second wireless communication receiver. In some embodiments, the third coil 662 in the second portion 141″ comprises the second wireless energy receiver and/or the second wireless communication receiver.

The second portion 141″ comprises a distal end 668 and a proximal end 670, here defined with respect to the connecting portion 142. In particular, the proximal end 668 is arranged closer to the connecting portion 142 and closer to the first portion 141′ when the device 100 is assembled. In the illustrated embodiment, the third coil 662 is arranged at the proximal end 668 of the second portion 141″. Such placement of the third coil 662 may provide for that energy and/or communication signals received by the third coil 662 will not be attenuated by internal components of the second portion 141″ when being received from the first portion 141′.

The first portion 141′ may comprise a first controller 102a connected to the first coil 658, second coil 660, and/or third coil 662. The second portion 141″ may comprise a second controller 102b connected to the first coil, 658, second coil 660, and/or third coil 662.

In the illustrated embodiment, the first portion 141′ comprises a first energy storage unit 304a connected to the first wireless energy receiver 308a, i.e. the first coil 658. The second portion comprises a second energy storage unit 304b connected to the second wireless energy receiver 308b, i.e. the third coil 662. Such an energy storage unit may be a solid-state battery, such as a thionyl-chloride battery.

In some embodiments, the first coil 658 is configured to receive energy transmitted wirelessly by the external wireless energy transmitter and store the received energy in the first energy storage unit 304a. Furthermore, the first coil 658 and/or the second coil 660 may be configured to wirelessly transmit energy stored in the first energy storage unit 304a to the third coil 662, and the third coil 662 may be configured to receive energy transmitted wirelessly by the first coil 658 and/or the second coil 660 and store the received energy in the second energy storage unit 305b.

The first energy storage unit 304a may be configured to store less energy than the second energy storage unit 304b, and/or configured to be charged faster than the second energy storage unit 304b.

Hereby, charging of the first energy storage unit 304a may be relatively quick, whereas transfer of energy from the first energy storage unit 304a to the second energy storage unit 304b may be relatively slow. Thus, a user can quickly charge the first energy storage unit 304a, and will not during such charging be restricted for a long period of time by being connected to an external wireless energy transmitter, e.g. at a particular location. After having charged the first energy storage unit 304a, the user may move freely while energy slowly transfers from the first energy storage unit 304a to the second energy storage unit 304b, via the first and/or second coil and the third coil.

FIGS. 137bb and 137cc illustrate a gear arrangement and magnetic coupling for coupling the implantable energized medical device to an implant (or element) exerting force on a body part, and in particular a gear arrangement for transferring mechanical movement through an outer housing of the device or an outer housing of the second portion 141″.

The housing 484 of the device or second portion 141″ may be present in some embodiments of the device. In such embodiments, the housing 484 is configured to enclose, at least, the controller (not shown), motor MO, any receivers and transmitters if present (not shown), and any gear arrangements G, G1, G2 if present. Hereby, such features are protected from bodily fluids. The housing 484 may be an enclosure made from one of or a combination of: a carbon-based material (such as graphite, silicon carbide, or a carbon fiber material), a boron material, a polymer material (such as silicone, Peek®, polyurethane, UHWPE or PTFE,), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability, such that migration of fluid through the walls of the enclosure is prevented.

The implantable energized medical device may comprise at least part of a magnetic coupling, such as a magnetic coupling part 490a. A complementary part of the magnetic coupling, such as magnetic coupling part 490b, may be arranged adjacent to the device 100, so as to magnetically couple to the magnetic coupling part 490a and form the magnetic coupling. The magnetic coupling part 490b may form part of an entity not forming part of the device 100. However, in some embodiments the second portion 141″ comprises several chambers being hermetically sealed from each other. Such chambers may be coupled via a magnetic coupling as discussed herein. The magnetic coupling 490a, 490b provide for that mechanical work output by the device 100 via e.g. an electric motor can be transferred from the device to e.g. an implant (or element) configured to exert force on a body part of a patient. In other words, the magnetic coupling 490a, 490b provides for that mechanical force can be transferred through the housing 484.

The coupling between components, such as between a motor and gear arrangement, or between a gear arrangement and a magnetic coupling, may be achieved by e.g. a shaft or the like.

In some embodiments, for example as illustrated in FIG. 137bb, a force output of a motor M in the second portion 141″ is connected to the magnetic coupling part 490a. The magnetic coupling part 490a transfers the force output from the motor M to the magnetic coupling part 490b, i.e. via the magnetic coupling 490a, 490b. The force output transferred via the magnetic coupling 490a, 490b here has a torque T1, which is substantially the same torque as delivered by the motor MO. The magnetic coupling part 490b is connected to a gear arrangement G, located external to the device, for example in a medical implant configured to exert force on a body part, or intermediate to a medical implant configured to exert force on a body part. The gear arrangement G is configured to increase the torque of the force delivered via the magnetic coupling 490a, 490b to deliver a force with torque T2 being higher than torque T1 to a medical implant. Consequently, low torque may be provided by the motor MO, i.e. a relatively small force with high angular velocity, which is transferred via the magnetic coupling 490a, 490b before the torque is increased via gear arrangement G to achieve a relatively large force with low angular velocity. Hereby, the magnetic coupling 490a, 490b may utilize relatively weak magnetic forces to transfer the mechanical work through the housing 484 of the device without the risk of slipping between the magnetic coupling parts 490a, 490b.

In some embodiments, for example as illustrated in FIG. 137cc, a force output of a motor M in the second portion 141″ is connected to a first gear arrangement G1, which in turn is coupled to the magnetic coupling part 490a. The motor M here provides a mechanical force with torque TO. The magnetic coupling part 490a transfers the force output from the motor M to the first gear arrangement G1. The first gear arrangement G1 is configured to increase the torque of the force delivered from the motor M to deliver a force with a higher torque T1 to the magnetic coupling 490a, 490b. The magnetic coupling part 490a transfers the force with torque T1 to the magnetic coupling part 490b. The magnetic coupling part 490b is connected to a second gear arrangement G2, located external to the device, for example in a medical implant configured to exert force on a body part, or intermediate to a medical implant configured to exert force on a body part. The second gear arrangement G2 is configured to increase the torque of the force delivered via the magnetic coupling 490a, 490b to deliver a force with torque T2 being higher than torque T1, and thus higher than torque TO, to a medical implant.

Consequently, low torque may be provided by the motor MO, i.e. a relatively small force with high angular velocity. The torque of the force provided by the motor M is then increased by the first gear arrangement G1, before the force is transferred via the magnetic coupling 490a, 490b. The torque of the force transferred via the magnetic coupling 490a, 490b is then yet again increased via the second gear arrangement G2 to achieve a relatively large force with low angular velocity. Hereby, the magnetic coupling 490a, 490b may utilize relatively weak magnetic forces to transfer the mechanical work through the housing 484 of the device without the risk of slipping between the magnetic coupling parts 490a, 490b. Furthermore, since some of the torque increase is made within the second portion 141″, and a remaining portion of the torque increase is made external to the device and the second portion 141″, the gear arrangements G1, G2 may be sized and configured appropriately to share the work of increasing the torque.

FIG. 137dd schematically illustrates an energy storage 304b connected to a wireless energy transmitter 308. The energy storage 304b and the wireless energy transmitter 308 are arranged in one portion or chamber of the second portion 141″. Furthermore, a wireless energy receiver 308e is arranged in another portion or chamber of the second portion 141″. The portions or chambers may be separated or defined by respective housings, external walls and/or internal walls 484a, 484b. The wireless energy transmitter 308d is configured to wirelessly transmit energy to the wireless energy receiver 308e.

Hereby, an internal energy transfer is achieved within the second portion 141″. The wireless energy transmitter 308d and wireless energy receiver 308e may comprise one or more coils, respectively. The wireless energy receiver 308e may be connected to a further energy storage 680 arranged within the second portion 141″. Such energy storage 680 may be connected to a medical implant, such that the energy storage 680 can deliver energy to the medical implant. In some embodiments however, the wireless energy receiver 308e is directly connected to a medical implant to deliver energy directly to the medical implant, thus omitting the energy storage 680.

FIG. 137ee shows a frontal view of the abdomen of a patient when a medical device 10 configured to exert force on a body portion of the patient has been implanted. Here, the medical device is configured to exert a force on the stomach of the patient. The medical device 10 is in the embodiment shown in FIG. 137ee operated by a remote unit 100. This is however only an example of a remote unit for operation of the medical device 10 and it is clear that any of the embodiments of remote units disclosed herein can be implanted and connected in the manner described with reference to FIG. 137ee. The remote unit 100 comprises a first portion 141′, a second portion 141″, and a connecting portion 142, mechanically connecting the first and second portions 141′, 141″. The first and second portions will hereinafter be interchangeably used with the terms “first unit” and “second unit” respectively. The second unit 141″ is in the embodiment shown in FIG. 137ee placed on the inside of muscular tissue MT of the abdominal wall AW of the patient, whereas the first unit 141′ is placed on the outside of the muscular tissue MT of the abdominal wall AW, in the subcutaneous tissue ST. As such, the connecting portion 142 travels through a created hole in, or natural orifice between, the muscles of the muscular tissue MT. A cross-sectional area of the connecting portion 142, in a plane in the extension of the muscular tissue MT is smaller than a cross-sectional area of the first and second units 141′, 141″, parallel to the cross-sectional area of the connecting portion 142. The cross-sectional areas of the first and second units 141′, 141″ are also larger than the created hole or natural orifice though which the connecting portion 142 is placed. As such, the first and second units 141′, 141″ are unable to pass through the created hole or natural orifice and is as such fixated to the muscular tissue MT of the abdominal wall.

This enables the remote unit 100 to be suspended and fixated to the muscle tissue MT of the abdominal wall AW.

In the embodiment shown in FIG. 137ff, the connecting portion 142, is a connecting portion 142 having a circular cross-section and an axial direction AD extending from the first unit 141′ to the second unit 141″. The plane in the extension of the muscular tissue MT, is in the embodiment of FIG. 137ff perpendicular to the axial direction AD of the connecting portion 142 extending from the first unit 141′ to the second unit 141″.

In the embodiment of FIG. 137ee, a controller is placed in the second unit 141″, and an implantable energy storage unit is placed in the second unit 141″. The controller and the implantable energy storage unit are electrically connected to each other by means of a lead running in the connecting portion 142, such that electrical energy and communication can be transferred from the first portion 141′ to the second portion 141″, and vice versa. In the embodiment of FIG. 137ee, the first portion 141′ further comprises a wireless energy receiver for receiving wireless energy for charging the implantable energy storage unit and/or for powering the medical device 10, and a transceiver for receiving and/or transmitting wireless signals to/from the outside the body.

The abdominal wall AW is most locations generally formed by a set of layers of skin, fat/fascia, muscles and the peritoneum. The deepest layer in the abdominal wall AW is the peritoneum PT, which covers many of the abdominal organs, for example the large and small intestines. The peritoneum PT is a serous membrane composed of a layer of mesothelium supported by a thin layer of connective tissue and serves as a conduit for abdominal organ's blood vessels, lymphatic vessels, and nerves. The area of the abdomen enclosed by the peritoneum PT is called the intraperitoneal space. The tissue and organs within the intraperitoneal space are called “intraperitoneal” (e.g., the stomach and intestines). The tissue and organs in the abdominal cavity that are located behind the intraperitoneal space are called “retroperitoneal” (e.g., the kidneys), and tissue and organs located below the intraperitoneal space are called “subperitoneal” or “intraperitoneal” (e.g., the bladder).

The peritoneum PT is connected to a layer of extraperitoneal fat EF which is connected to a layer or transversalis fascia TF. Connected to the transversalis fascia TF, at the area of the abdominal wall AW at which the section is extracted, is muscle tissue MT separated by layers of deep fascia DF. The deep fascia DF between the layers of muscle is thinner than the transversalis fascia TF and the Scarpa's fascia SF placed on the outside of the muscle tissue MT. Both the transversalis fascia TF and the Scarpa's fascia SF are relatively firm membranous sheets. At the area of the abdominal wall AW at which the section is extracted, the muscle tissue MT is composed of the transverse abdominal muscle TM (transversus abdominis), the internal oblique muscle IM (obliquus internus) and the external oblique muscle EM (obliquus externus). In other areas of the abdominal wall AW, the muscle tissue could also be composed of the rectus abdominis and the pyramidalis muscle.

The layer outside of the muscle tissue MT, beneath the skin SK of the patient is called subcutaneous tissue ST, also called the hypodermis, hypoderm, subcutis or superficial fascia. The main portion of the subcutaneous tissue ST is made up of Camper's fascia which consists primarily of loose connective tissue and fat. Generally, the subcutaneous tissue ST contains larger blood vessels and nerves than those found in the skin.

Placing the remote unit 100 at an area of the abdomen is advantageous as the intestines are easily displaced for making sufficient room for the remote unit 100, without the remote unit 100 affecting the patient too much in a sensational or visual way. Also, the placement of the remote unit 100 in the area of the abdomen makes it possible to fixate the remote unit 100 to the muscle tissue MT of the abdomen for creating an attachment keeping the remote unit 100 firmly in place. In the embodiment shown in FIG. 137ee, the second portion 141″ of the remote unit 100 is placed on the left side of the patient in between the peritoneum PT and the muscle tissue MT. The first portion 141′ is placed in the subcutaneous tissue ST between the muscle tissue MT and the skin SK of the patient. Placing the first portion 141′ subcutaneously enables easy access to the first portion 141′ for e.g. wireless communication using a wireless transceiver placed in the first portion 141′, wireless charging of an implantable storage unit using a wireless energy receiver placed in the first portion 141′, injection of a hydraulic fluid (relevant when the operation device is a hydraulic operation device), into an injection port placed in the first portion 141′, manual manipulation of for example a push button placed in the first portion 141′, or maintenance or replacement of the first portion 141′ via a small incision in the skin SK at the first portion 141′.

In the embodiment shown in FIG. 137ee, the flexible wires 135 running inside of protective a cover 136 transports linear mechanical force from the remote unit 100 to the main portion MP of the medical device 10. The flexible wires 135 run between the peritoneum PT and the muscle tissue MT vertically until the flexible wires 135 reaches the height of the main portion MP of the medical device 10. At this height, the wires 135 enters the peritoneum PT and travels substantially horizontally to the main portion MP of the medical device 10. As such, the flexible wire 135 is placed inside of the intraperitoneal space for as short distance as possible which reduces the risk that implanted, foreign body, elements disturb the intraperitoneal organs, reducing the risk of damage to organs, and reducing the risk that foreign body elements cause ileus.

In the embodiment shown in FIG. 137ee, the connecting portion 142 connects the first and second portions 141′, 141″ through three layers of muscle tissue MT, namely tissue of the transverse abdominal muscle TM, the internal oblique muscle IM and the external oblique muscle EM. In alternative embodiments, it is however conceivable that the second portion 141″ is placed in between layers of muscle, such as between tissue of the transverse abdominal muscle TM, the internal oblique muscle IM, or between the internal oblique muscle IM and the external oblique muscle EM. As such, it is conceivable that in alternative embodiments, the connecting portion 142 connects the first and second portions 141′, 141″ through two layers of muscle tissue MT, or through one layer of muscle tissue MT.

In alternative embodiments, it is furthermore conceivable that the first portion 141′ is placed in between layers of muscle, such as between tissue of external oblique muscle EM and the internal oblique muscle IM, or between the internal oblique muscle IM and the transverse abdominal muscle TM.

In embodiments in which the medical device exerting a force on a body part is hydraulically remotely operable (such as via a remote unit comprising a pump as further described with reference to

FIG. 137gg), the flexible wires 135 running inside of protective a cover 136 for transporting linear mechanical force from the remote unit 100 to the main portion MP shown in FIG. 137ee is replaced by conduits (109 in FIG. 137gg) for conducting hydraulic fluid for transferring force from a portion of the hydraulic operation device placed in the remote unit 100 to a portion of the operation device placed in the main portion MP of the medical device 10 hydraulically.

FIG. 137ff shows a frontal view of the abdomen of the patient when a medical device 10 for exerting a force on a body part has been implanted. Here, the medical device 10 is configured to affect the flow of urine of the patient. The medical device 10 is in the embodiment shown in FIG. 137ff operated by a remote unit 100 and it is clear that any of the embodiments of remote units disclosed herein can be implanted and connected in the manner described with reference to FIG. 137ff. The remote unit 100 comprises a first portion 141′, a second portion 141″, and a connecting portion 142, mechanically connecting the first and second portion 141′, 141″. The second portion 141″ is in the embodiment shown in FIG. 137ff placed on the inside of muscular tissue MT of the abdominal wall AW of the patient, whereas the first portion 141′ is placed on the outside of the muscular tissue MT of the abdominal wall AW, in the subcutaneous tissue ST. As such, the connecting portion 142 travels through a created hole in, or natural orifice between, the muscles of the muscular tissue MT. A cross-sectional area of the connecting portion 142, in a plane in the extension of the muscular tissue MT is smaller than a cross-sectional area of the first and second portions 141′, 141″, parallel to the cross-sectional area of the connecting portion 142. The cross-sectional areas of the first and second portions 141′, 141″ are also larger than the created hole or natural orifice though which the connecting portion 142 is placed. As such, the first and second portions 141′, 141″ are unable to pass through the created hole or natural orifice and is as such fixated to the muscular tissue MT of the abdominal wall. This enables the remote unit 100 to be suspended and fixated to the muscle tissue MT of the abdominal wall AW.

In the embodiment shown in FIG. 137ff, the second portion 141″ is configured to connect to the medical implant 10 in a cadial direction, i.e. a distal end of the second portion 141″ comprises a connecting interface for delivering mechanical force, fluid, energy and/or for transmitting or receiving communication signals, to and from the medical implant 10.

In the embodiment shown in FIG. 137ff, the flexible wires 135 running inside of protective a cover 136 transports linear mechanical force from the remote unit 100 to the medical device 10. The flexible wires 135 run between the peritoneum PT and the muscle tissue MT vertically until the flexible wires 135 reaches the area of the urinary bladder U in the subperitoneal space below the intraperitoneal space. As such, the flexible wire 135 never needs to enter the intraperitoneal space which reduces the risk that implanted, foreign body, elements disturbs the intraperitoneal organs, reducing the risk of damage to organs, and reducing the risk that foreign body elements cause ileus.

In the embodiment shown in FIG. 137ff, the connecting portion 142 connects the first and second portions 141′, 141″ though three layers of muscle tissue MT, namely tissue of the transverse abdominal muscle TM, the internal oblique muscle IM and the external oblique muscle EM. In alternative embodiments, it is however conceivable that the second portion 141″ is placed in between layers of muscle, such as between tissue of the transverse abdominal muscle TM, the internal oblique muscle IM, or between the internal oblique muscle IM and the external oblique muscle EM. As such, it is conceivable that in alternative embodiments, the connecting portion 142 connects the first and second portions 141′, 141″ through two layers of muscle tissue MT, or through one layer of muscle tissue MT.

In alternative embodiments, it is furthermore conceivable that the first portion 141′ is placed in between layers of muscle, such as between tissue of external oblique muscle EM and the internal oblique muscle IM, or between the internal oblique muscle IM and the transverse abdominal muscle TM.

With reference to FIGS. 137gg-137rr, hydraulic pumps will be described. Such hydraulic pumps may be placed in a second portion of an implantable energized medical device as described herein. However, it is also possible to place an electrical motor in the second portion of such a device for providing mechanical work to an external implant, without the use of hydraulic pumps or any hydraulic means.

FIG. 137gg shows a cross-sectional view of an electrical motor M in combination with a gear system G for propulsion of a hydraulic pump 104. The hydraulic pump 104 may be placed in a remote unit, such as an implantable energized medical device, and in particular in a second portion of such a device. The electrical motor M is connected to the controller 300 which in turn is connected to an energy storage unit 40. The energy storage unit 40 may be a battery, a chargeable battery or a capacitor by means of which energy can be stored in the body of the patient. The energy storage unit 40 may comprise an energy storage unit and wireless charging components, such as wireless receiver and transmitters for receiving and transmitting energy wirelessly.

The controller 300, the energy storage unit 40 and the motor M and gear system G may be enclosed by a housing 484 such that the controller 300 is protected from bodily fluids. The housing 484 may be an enclosure made from one of or a combination of: a carbon based material (such as graphite, silicon carbide, or a carbon fiber material), a boron material, a polymer material (such as silicone, Peek®, polyurethane, UHWPE or PTFE,), a metallic material (such as titanium, stainless steel, tantalum, platinum, niobium or aluminum), a ceramic material (such as zirconium dioxide, aluminum oxide or tungsten carbide) or glass. In any instance the enclosure should be made from a material with low permeability, such that migration of fluid through the walls of the enclosure is prevented.

Turning now to the hydraulic pump 104 shown in FIG. 137gg. In the embodiment shown in FIG. 137gg, the force output 449 of the gear system G is threaded 449t and engages a correspondingly threaded portion 451t of the movable wall 451 such that the rotating force created by the motor M and gear system G is transferred to a linear force moving the movable wall 451. The threaded force output 449 is enclosed by pleated bellows portions 452 both above and below the movable wall 451 such that the threaded force output 449 is protected from the fluid in the lumens of the reservoirs 107a, 107b. The reservoirs 107a, 107b has a common moveable wall 451 for changing the volume of the implantable fluid reservoirs 107a, 107b and thereby increasing fluid in the first fluid reservoir 107a simultaneously with decreasing fluid in the second fluid reservoir 107b and vice versa. The peristaltic pump is a sealed pump which means that fluid will not leak through the pump even at standstill. As the peristaltic pump is a sealed pump no additional valve is needed to keep the fluid through the fluid conduits 109′, 109″ closed.

The movable wall pump 104 of FIG. 137gg is a sealed pump which means that fluid will not leak through the pump even at standstill. As the movable wall pump 104 is a sealed pump, no additional valve is needed to keep the fluid through the fluid conduits 109′, 109″ closed.

FIG. 137hh shows a cross-sectional view of a hydraulic pump comprising two expandible reservoirs 107a, 107b. The hydraulic pump 104 may be placed in a remote unit. The hydraulic pump 104 of FIG. 137hh comprises an encapsulated motor M, gear system G, controller 300 and energy storage unit being identical to that described with reference to FIG. 137gg. Turning to the hydraulic pump 104, the force output 449 is, in the embodiment described in FIG. 137hh a hollow shaft equipped with inner threads (not shown) adapted to engage outer threads 453t of a threaded member 453, such that the interaction between the hollow shaft 449 and the threaded member 453 transforms the radially rotating force generated by the motor M and the gear system G, to a linear force. The threaded member 453 is connected to a radially extending engaging member 454 adapted to engage the first and second reservoirs 107a, 107b containing a hydraulic fluid. The reservoirs 107a, 107b may be fixated to the radially extending engaging members 454, for example by means of an adhesive, such that the reservoirs 107a, 107b are forced to expand when the radially extending engaging member 454 is moved upwards in the expanding direction of the reservoirs 107a, 107b. The first reservoir 107a is connected to a first fluid conduit and the second reservoir 107b is connected to a second fluid conduit 109″. The embodiment shown in FIG. 137hh further comprises a pleated bellows portions 452 for encapsulating and protecting the force output 449 and the threaded member 453 from bodily fluids. The reservoirs 107a, 107b are preferably made from medical grade implantable silicone or Parylene® coated medical grade implantable silicone, but may in alternative embodiments be made from another resilient material such as NBR, Hypalon, Viton, PVC, EPDM, Polyurethane or Natural Rubber. When the reservoirs 107a, 107b are compressed and expanded they function as hydraulic pumps for moving hydraulic fluid any of the hydraulic embodiments herein.

FIG. 137ii shows a cross-sectional view of a hydraulic pump 104 similar to the hydraulic pump or the embodiment of FIG. 137hh. In the embodiment of FIG. 137ii, the hydraulic pump 104 comprises one expandible reservoir 107. The hydraulic pump 104 comprises an encapsulated motor M, gear system G, controller 300 and energy storage unit 40. The motor M is configured to generate force in a radial direction by rotation of the force output in the form of a shaft 481. The shaft 481 is equipped with outer threads 481t adapted to engage inner threads 483t of a compression member 483, such that the interaction between the threaded shaft 481, 481t and the threaded portion 483t of the compression member 483 transforms the radially rotating force generated by the motor M and the gear system G, to a linear force acting in the axial direction of the shaft 481, and thus makes up a transmission T. The axial force acts on the compression member 483 which engages a first resilient wall 102a of the compressible reservoir 107 for compressing the compressible reservoir 107 and thus increasing the pressure on a hydraulic fluid in the compressible reservoir 107. The compression member 483 may be fixated to the first resilient wall portion 102a by means of an adhesive, such that the reservoir 107 is forced to expand when the compression member 483 moves in the expanding direction of the reservoir 107. The reservoir 107 is connected to a fluid conduit (not shown) for conducting hydraulic fluid from the compressible reservoir to the and from the reservoir 107. The reservoir 107 is preferably made from medical grade implantable silicone or Parylene® coated medical grade implantable silicone, but may in alternative embodiments be made from another resilient material such as NBR, Hypalon, Viton, PVC, EPDM, Polyurethane or Natural Rubber. When the reservoir 107 is compressed and expanded it functions as hydraulic pump for moving hydraulic fluid to and from a medical implant configured to exert a force on a body part.

The hydraulic pump 104 further comprises at least one bearing 482 for the shaft 481 placed between the gear system G and the compressible reservoir 107. The bearing 482 is configured to withhold at least half of the force in the axial direction, for reducing the axial load on the motor M and the gear system G which is caused by the compression of the reservoir 107. In the embodiment shown in

FIG. 137ii, the bearing 482 is a ball bearing, but in other embodiments the bearing may comprise a roller bearing or a plain bearing preferably including a self-lubricating material such as PTFE or HDPE.

The gear system G is connected to the motor M and placed between the motor M and transmission T and adapted to receive mechanical work via the shaft 481 having a force and a velocity, and output mechanical work having a stronger force and a lower velocity. The compressible reservoir 107 comprises a first resilient wall portion 102a and a second resilient wall portion 102b, wherein the first resilient wall portion 102a is more resilient than the second resilient wall portion 102b.

In alternative embodiments, the compression member 483 may be directly connected to the first resilient wall portion 102a, and in such embodiments, the threaded portion 483t may be integrated in the first resilient wall portion 102a.

In the embodiment shown in FIG. 137ii, the hydraulic pump 104 further comprises a pressure sensor 106 connected to the compressible reservoir 107 and configured to sense the pressure in the compressible reservoir 107. The pressure sensor 106 is integrated in, and placed on the outside of, the second resilient wall portion 102b of the compressible reservoir 107. It may be important to measure strain or pressure in or exerted by the medical device, as too high strain or pressure risks hampering the blood flow to the tissue of a stomach wall, which in the long term could lead to damage of the tissue and in the worst-case lead to necrosis.

The compressible reservoir 107 in the embodiment shown in FIG. 137ii comprises a first and second resilient wall portion 102a, 102b in the form of a first and second circular diaphragm 102a, 102b.

The first resilient wall portion 102a has a convex shape facing the compression member 483, and the second resilient wall portion 102b has a convex shape facing away from the compression member 483 and a lumen is formed between the two diaphragms 102a, 102b, and being enclosed by the concave surfaces of the diaphragms 102a, 102b. The first resilient wall portion 102a is configured to be compressed and thus inverted, such that the part of the first resilient wall portion 102a facing the compression member 483 assumes a concave shape facing the compression member 483, and as such, a convex shape is formed towards the lumen of the compressible reservoir 107. The inverted, convex, portion of the first resilient wall portion 102a thus enters the concave shape of the second resilient wall portion 102b. The portion of the compression member 483 configured to engage the first resilient wall portion 102a comprises a convex portion for facilitating the inversion of the convex portion of the first resilient wall portion 102a. In the embodiment shown in FIG. 137ii, the first resilient wall portion 102a is more resilient than the second resilient wall portion 102b such that the compressible reservoir 107 can create a suction when the compression member 483 moves in the direction away from the compressible reservoir 107 thus enabling the compressible reservoir 107 to expand. In the embodiment shown in FIG. 137ii, a major portion of the first resilient wall portion is made from a material having a modulus of elasticity (E) which is less than 70% or the modulus of elasticity (E) of the material of a major portion of the second resilient wall portion 102b. In alternative embodiments, it is conceivable that the first and second resilient wall portions 102a, 102b are made from the same material, but with the second resilient wall portion 102b being more than 1.5 times as thick as the first resilient wall portion 102a. In the embodiment shown in FIG. 137ii, the two diaphragms 102a, 102b are pressed against each other, for creating the sealed lumen between the first and second diaphragm, by means of a fixation ring 485, which is screwed into the housing 484.

In the embodiment shown in FIG. 137ii, the hydraulic pump further comprises a shaft sealing 486, which is a sealing engaging the shaft and thus creating a seal between the portion of the pump housing 484 comprising the motor M, gear system G, energy storage unit 40 and controller 300, and the portion of the pump housing 484 comprising the compressible reservoir 107. The seal reduces the risk that hydraulic fluid that may leak from the compressible reservoir 107 will come in contact with any of the motor M, gear system G, energy storage unit 40 and/or controller 300. In the embodiment shown in

FIG. 137ii, the shaft sealing comprises a spring-loaded PTFE sealing 486. A spring engages the housing 484 of the hydraulic pump 104 and the PTFE sealing for creating a constant elastic pressure between the sealing and the shaft 481 which ensures a self-lubricating tight seal. In alternative embodiments, the spring may be replaced by a different type of elastic element, such as an elastic element made from an elastomer. In alternative embodiment, the shaft sealing 486 could be a shaft sealing made from another self-lubricating material such as HDPE.

The hydraulic pump 104 of FIG. 137ii is enclosed by a pump housing 484, which in the embodiment shown in FIG. 137ii is a titanium housing 484. In alternative embodiments, the housing could be made from another medical grade metal alloy, such as medical grade stainless steel or could comprise a ceramic material such as zirconium carbide, or a stiff medical grade polymer material such as Ultra-high-molecular-weight polyethylene (UHMWPE) or Polytetrafluoroethylene (PTFE) or a thermoplastic polyester such as polylactide (PLA). The housing could also comprise at least one composite material, such as any combination of metallic/ceramic and polymer materials or a polymer material reinforced with organic or inorganic fibers, such as carbon or mineral fibers.

FIG. 137jj shows a cross-sectional view of a hydraulic pump 104 similar to the hydraulic pump of the embodiment of FIG. 137ii. In the embodiment of FIG. 137jj, the hydraulic pump comprises one expandible reservoir 107. The hydraulic pump 104 comprises a housing 484 comprising a first and a second chamber C1, C2 separated from each other by a barrier 484′. Just as in the embodiment of FIG. 137ii, the first chamber C1 comprises the motor M configured for transforming electrical energy to mechanical work and the gear system gear system G adapted to receive mechanical work having a first force and first velocity, and output mechanical work having a different second force and a different second velocity, such that the high velocity movement supplied by the electrical motor M is transformed to low velocity movement with increased force. The output mechanical work having the different second force and different second velocity acts on a shaft 481 which transfers the force to a magnetic coupling 490a, 490b for transferring mechanical work from the motor M to an actuator in the form of a compression member 483 for compressing the expandible reservoir 107 for pressing a hydraulic fluid through the conduit 109. The magnetic coupling 490a, 490b comprises a first disc shaped member 490a mounted to the shaft 481 such that the first disc shaped member 490a rotates along with the shaft 481. The shaft 481 is supported by ball bearings 482 assisting in the centering of the shaft 481.

The first disc shaped member 490a comprises magnets (or a material susceptible to magnetic fields) 491 evenly distributed axially in a circular formation on the distal surface of the first disc shaped member 490a.

The barrier 484′ separates the first chamber C1 of the housing 484 from the second chamber C2 of the housing. In the embodiment shown in FIG. 137jj, the barrier 484′ is made from the same material as the outer wall of the housing 484, i.e. medical grade titanium. In the embodiment shown in FIG. 137jj the barrier is materially integrated with the portion of the outer wall of the housing 484 enclosing the second chamber C2. However, in other embodiments it is equally conceivable that the barrier is materially integrated with the portion of the outer wall of the housing 484 enclosing the first chamber C1. In any event, the purpose is the both the first and second chambers C2 should be hermetically enclosed and separated from each other.

The second part of the magnetic coupling comprises a second disc shaped member 490b positioned in the second chamber C2 and held in place by a ball bearing 482b being fixated to the inside of the wall of the housing 484 enclosing the second chamber C2 by means of an internal wall portion 498. The second disc shaped member 490b comprises magnets (or a material susceptible to magnetic fields) 491b evenly distributed in a circular formation axially on the distal surface of the first disc shaped member 490b. The magnets 490b of the second disc shaped member 490b are configured to be magnetically connected to the magnets 491a of the first disc shaped member 490a such that the second disc shaped member 490b is dragged by the first disc shaped member 490a by means of the magnetic connection. As such, force from the motor M is transferred from the first hermetically enclosed chamber C1 to the second hermetically enclosed chamber C2.

The second disc shaped member 490b comprises a threaded shaft which is configured to be placed in and engage with a sleeve of a compression member 483. The sleeve of the compression member 483 comprises inside threads 483t for creating a transmission T that transforms the radially rotating force generated by the motor M and the gear system G, to a linear force acting in the axial direction of the shaft 481, and thus makes up a transmission T.

The compression member 483 is a disc shaped element having a distal surface engaging a first resilient wall portion 102a of the reservoir 107 for moving the first resilient wall portion 102a and thereby compressing the reservoir 107. The periphery of the compression member 483 comprises a flange 483f extending towards the first chamber C1 in the proximal direction creating a lateral surface area towards the housing 484. The lateral surface of the flange 483f is configured to engage the first resilient wall portion 102a for creating a rolling crease of the first resilient wall portion 102a. The disc shaped compression member 483 is rigid and made from titanium, just as the rest of the housing 484.

That the compression member 483 is rigid makes the reservoir 107 stiff which ensures that the fluid amount in the medical implant connected to the reservoir 107 remains the same even as the pressure exerted on the medical implant increases.

The reservoir 107 is further enclosed by a second wall portion 102b which is a rigid titanium wall portion through which the conduit 109 enters the reservoir 107. Compression of the reservoir 107 thus forces the fluid from the reservoir through the conduit 109. The housing 484 further comprises a transfer channel 478 creating a fluid connection between the second chamber C2 and a portion of the second chamber C2′ placed more distally. The transfer channel ensures that the pressure is the same in the second chamber C2 and distal portion of the second chamber C2′. The distal portion C2′ of the second chamber C2 comprises an expansion portion comprising a resilient membrane 495 configured to move to alter the volume of the distal portion C2′ of the second chamber C2 for compensating for the changes to the volume of the reservoir 107 which is created by the movement of the first resilient wall portion 102a of the reservoir 107. As such, the pressure in the second chamber C2 will be substantially constant. The resilient membrane 495 is in the embodiment shown in FIG. 137jj made from a medical grade elastic silicone material but may in alternative embodiments be made from another biocompatible polymer material, such as polyurethane.

The hydraulic pump of FIG. 137jj further comprises a pressure sensor 106 placed on the first resilient wall portion 102a of the chamber 107 for sensing the pressure in the chamber 107. The sensor 106, is connected to electrical conduits 493 for transferring an electrical sensor signal from the pressure sensor 106 to the controller 300. The electrical conduits 493 passes from the second chamber C2 to the first chamber C1 through an electrically insulating ceramic grommet 494 integrated in the barrier 484′ wall such that the conduits 493 can pass the barrier 484′ without being further insulated which enables the conduits 493 to pass through the barrier 484′ whilst the barrier hermetically separates the first chamber C1 from the second chamber C2. It may be important to measure strain or pressure in or exerted by the medical device, as too high strain or pressure risks hampering the blood flow to the tissue of the stomach wall, which in the long term could lead to damage of the tissue and in the worst-case lead to necrosis.

A first portion 109a of the fluid conduit is connected to an implantable hydraulic force transfer device 496 comprising a first chamber V1 configured to house a first fluid, and as such the first portion 109a of the fluid conduit forms a fluid inlet into the first chamber V1. The first chamber V1 is in connection with a movable wall portion 497 for varying the size of the first chamber V1. The movable wall portion 497 is in turn connected to a second chamber V2 configured to house a second fluid. The second chamber comprises an outlet formed by a second portion 109b of the fluid conduit. The second portion 109b of the fluid conduit fluidly connects the second chamber C2 to a conduit (139) in any of the hydraulic embodiments described herein. As such, the implantable hydraulic force transfer device 496 transfers hydraulic force from a remote unit to the main portion MP of a medical device configured to exert a force on a body part without mixing the first and second fluids.

In the embodiment shown in FIG. 137jj, the implantable hydraulic force transfer device 496 comprises a cylinder-shaped housing in which the piston-like movable wall portion 497 moves linearly. The piston-like movable wall portion 497 seals against the inner side of the wall of the cylinder-shaped housing such that the first and second chambers V1, V2 remains separated. The implantable hydraulic force transfer device 496 enables the system to have a first fluid in the compressible reservoir 107 and in the first chamber V1 of the implantable hydraulic force transfer device 496. This part of the system may be hermetically sealed in such a way that leakage is highly improbable, which enables this part of the system to use a fluid which cannot be allowed to escape into the body, such as an oil based fluid, such as a silicone oil. The second part of the system, comprising the second chamber C2 of the implantable hydraulic force transfer device 496, the second portion 109b of the fluid conduit, and the rest of the hydraulic operation device of the medical device (not shown) will have a second fluid which must be a biocompatible fluid as some level of leakage or diffusion may be hard to avoid. In the second part of the system the fluid could for example be an isotone aqueous fluid, such as a saline solution.

The housing 484 and the housing of the implantable hydraulic force transfer device 496 may be a titanium housing. However, it is equally conceivable that the housing is made from another biocompatible material such as a medical grade metal alloy, such as medical grade stainless steel or a ceramic material such as zirconium carbide, or a stiff medical grade polymer material such as Ultra-high-molecular-weight polyethylene (UHMWPE) or Polytetrafluoroethylene (PTFE) or a thermoplastic polyester such as polylactide (PLA).

In alternative embodiments, the magnetic coupling described with reference to FIGS. 137jj and 137kk could be used in connection with another type of pumps, such as the pumps described with reference to FIGS. 137gg and 137rr. In the alternative, the magnetic coupling could be used in connection with a gear pump. It is also conceivable that the magnetic coupling could be used in connection with a mechanical actuator configured to transfer mechanical force from the magnetic coupling to a medical device to exert a force on a body portion of a patient. The mechanical actuator could be an actuator configured to transfer a rotating force into a linear force, such as the transmission (T) described with reference to FIGS. 137gg-137nn.

FIG. 137kk shows a hydraulic pump in an embodiment similar to the embodiment shown in FIG. 137jj. One difference with the embodiment of FIG. 137kk in comparison to the embodiment of FIG. 137jj is that the first coupling part 490a′ comprises magnets 491a′ or material susceptible to magnetic fields which are placed radially along an outer periphery, on the lateral surface, of the cylinder-like first coupling part 490a′. The magnets 491a′ of the first coupling part 490a′ are magnetically connected to magnets 491b′ placed radially on the inner letteral surface of the cylinder-shaped second coupling part 490b′. The magnets 491a′, 491b′ of the first and second coupling parts 490a′, 490b′ are separated from each other by the barrier 484′. The second coupling part 490b′ is connected to a rotatable shaft which is supported by ball bearings 482b being fixated to the inside of the wall of the housing 484 enclosing the second chamber C2 by means of an internal wall portion 498. The rotatable shaft comprises a threaded portion which is configured to be placed in and engage with a sleeve of a compression member 483. The sleeve of the compression member 483 comprises inside threads 483t for creating a transmission T that transforms the radially rotating force generated by the motor M and the gear system G, to a linear force acting in the axial direction of the shaft 481, and thus makes up a transmission T.

Another difference between the embodiment shown in FIG. 137jj and the embodiment shown in FIG. 137kk is in the implantable hydraulic force transfer device 496. In the embodiment shown in FIG. 137kk, the implantable hydraulic force transfer device 496 comprises a movable wall portion 497′ in the form of a bellows with a pleated flexible wall portion which can be compressed and expanded. The material of the flexible wall portion could be an elastic material, such as an elastic polymer material or a substantially inelastic material such as a metal material forming a metal bellows which is mainly flexible due to its shape. In an alternative embodiment, the flexible wall portion can be purely elastic and thus be without the pleats, which means that the expansion and contraction of the reservoir is done purely based on the elasticity of the material in the flexible wall. The flexible movable wall portion 497′ encloses the first chamber V1 and keeps the chamber V1 completely separated from the chamber V2. The implantable hydraulic force transfer device 496 enables the system to have a first fluid in the compressible reservoir 107 and in the first chamber V1 of the implantable hydraulic force transfer device 496. This part of the system may be hermetically sealed in such a way that leakage is highly improbable, which enables this part of the system to use a fluid which cannot be allowed to escape into the body, such as an oil based fluid, such as a silicone oil. The second part of the system, comprising the second chamber C2 of the implantable hydraulic force transfer device 496, the second portion 109b of the fluid conduit, and the implantable hydraulic constriction element (not shown) will have a second fluid which must be a biocompatible fluid as some level of leakage or diffusion may be hard to avoid. In the second part of the system the fluid could for example be an isotone aqueous fluid, such as a saline solution.

FIG. 137mm shows an embodiment of a hydraulic pump 104 which is similar to the embodiment shown in FIG. 137ii. One difference in comparison to the embodiment of FIG. 137ii is that the compression member 483 has a flat circular surface engaging the first resilient wall portion 102a of the reservoir 107. The flat surface is bonded to the first resilient wall portion 102a such that the first resilient wall portion 102a moves along with the compression member 483. The compression member 483 has a diameter such that a distance 483d is created between the compression member 483 and the portion of the housing facing the compression member 483. The distance is slightly more than two times the thickness of the first resilient wall portion 102a, such that the first resilient wall portion 102a can be folded such that a rolling crease of the first resilient wall portion 102a is created which moves along with the compression member 483. The distance 483d is smaller than the radius (or half cross-sectional distance) of the compression member 483. The distance is 483d is also smaller than half the radius of the compression member 483. The first resilient wall portion 102a, towards the second chamber C2, being either folded or supported by the compression member means that ensures that the reservoir 107 will be substantially stiff which enables the fluid amount in an hydraulically operable medical device connected to the reservoir 107 to remain the same even as the pressure exerted on the hydraulically operable medical device increases.

The embodiment of FIG. 137mm differs from the embodiment of FIGS. 137jj and 137kk in that it only comprises a single chamber C1. The housing 484 of the hydraulic pump 104 of FIG. 137mm comprises an expansion portion placed in the proximal portion of the hydraulic pump 104 (on the right side of the hydraulic pump of FIG. 137mm). The expansion portion comprises a first and second resilient membrane 495a, 495b with a silicone oil filling the space formed between the first and second resilient membranes 495a, 495b. The oil between the first and second resilient membrane 495a, 495b reduces the risk of diffusion of fluids through the expansion portion. The first and second resilient membranes 495a, 495b are placed on two sides of a portion 484″ of the housing comprising a hole through which the fluid can travel as the expansion portion compensates for the changes to the volume of the reservoir 107 which is created by the movement of the first resilient wall portion 102a of the reservoir 107. As such, the pressure in the first chamber C1 will be substantially constant. The first and second resilient membranes 495a, 495b are in the embodiment shown in FIG. 137mm made from a medical grade elastic silicone material but may in alternative embodiments be made from another biocompatible polymer material, such as polyurethane.

Another aspect of having the housings of any of the embodiments herein, is that the atmospheric pressure that the patient exists in may vary. At sea level, the air pressure is about 101 kPa, in a commercial airplane at cruising altitude, the air pressure is about 80 kPa which is about the same as in Mexico City, whereas in La Paz, the highest situated city, air pressure is only 62 kPa. This difference in air pressure affects any gaseous fluid, such as the air present in the chamber C1 in the embodiment of

FIG. 137mm. The reduced atmospheric air pressure means that the gaseous fluid inside of the housing needs to be able to expand if the pressure in the housing should remain the same. If the pressure in the housing would increase 20%-40%, the motor would have to operate the hydraulic medical device against that pressure which would mean that the motor would have to be more powerful which would require more energy. As the expansion portion comprises a resilient membrane, the expansion portion allows the gaseous fluid in the housing to expand which at least reduces the pressure increase in the housing in response to a reduced atmospheric pressure.

FIG. 137nn differs from the embodiment of FIG. 137mm only in that the chamber C1 is completely filled with a liquid dielectric silicone oil. The liquid fluid could in the alternative be a synthetic single-phase liquid dielectric fluid, such as ElectroCool EC-100, from Engineered Fluids, or a 2-phase coolant such as Fluorinert or Novec from 3M. The fluid in the chamber C1 is non-conductive and as such does not risk damaging the electrical components placed in the chamber C1, such as the energy storage unit 40. In the embodiment shown in FIG. 137nn, the expandible reservoir 107, the conduit 109 and the medical device configured to exert force on the body portion of the patient forms the second chamber and second hydraulic system configured to comprise a second liquid which is a hydraulic liquid configured to transfer force. The second liquid may be an isotone aqueous liquid, such as a saline solution.

In the embodiment shown in FIG. 137nn, the first chamber comprises the motor M, the gear system G and the transmission T for transforming the rotating force generated by the motor M to a linear force for pressing on the expandible reservoir 107. Advantages with having the housing and the first chamber C1 entirely filled with a liquid fluid includes the liquid acting as a cooling agent for components that may produce heat, such as the controller 300, the energy storage unit 40, the motor M, gear system G, bearing 482 and transmission T, and as a lubricant for components that may require lubrication, such as the motor M, gear system G, bearing 482 and transmission T.

Just as in FIG. 137mm, the housing 484 of the hydraulic pump 104 comprises an expansion portion 495a, 484″, 495b placed in the proximal portion of the hydraulic pump 104 (on the right side of the hydraulic pump of FIG. 137nn), such that the housing can expand when the expandable reservoir 107 expands.

In alternative embodiments, the liquid filled first chamber C1 could be used in connection with another type of pump, i.e. the shaft 481 could be connected to another type of pump, such as the pumps described with reference to FIG. 137gg and 137pp, or a gear pump.

FIG. 137pp shows an embodiment of a hydraulic pump 104 which is similar to the embodiment shown in FIG. 137kk. The main difference with the embodiment shown in FIG. 137pp is that it made more compact as the gear system is integrated in the magnetic coupling. The magnetic coupling thus comprises a magnetic gear which transfers a week force with a high velocity into a stronger force with lower velocity. The magnetic coupling/gear comprises a first coupling part 490a′ fixated to the shaft 481 connected to the electrical motor M such that the first coupling part 490a′ rotates along with the electrical motor M. The first coupling part 490a′ comprises a first number of magnets 491a′, which in the embodiment shown in FIG. 137pp is 6 magnets, 3 with each polarity (3 pole pairs). The magnets are placed radially along an outer periphery, on the lateral surface, of the cylinder-like first coupling part 490a′. The second coupling part 490b′ comprises a second number of magnets 491b′, placed radially on the inner letteral surface of the cylinder-shaped second coupling part 490b′. In the embodiment shown in

FIG. 137pp the second coupling part 490b′ comprises 26 (twenty six) magnets, 13 (thirteen) with each polarity. Between the first coupling part 490a′ and the second coupling part 490b′ there is a stationary part, which is a portion of the barrier 484′. The stationary part comprises a plurality of intermediate ferromagnetic elements 499 thus placed between the first and second coupling parts 490a′, 490b′. The intermediate ferromagnetic elements 499 directs the concentration of the magnetic lines between the magnets 491a′, 491b′ of the first coupling part 490a′ and the second coupling part 490b′. The gear ratio between the first coupling part 490a′ and the second coupling part 490b′ is the number of magnetic pole pairs on the second coupling part 490a′ divided by the number of magnetic pole pairs on the second coupling part 490b′. In the embodiment shown in FIG. 137pp, the gear ratio is 13/3. The number of intermediate ferromagnetic elements 499 is equal to the sum of pole pairs on the first and second coupling parts 490a′, 490b′. In the embodiment shown in FIG. 137pp this means that the number of intermediate ferromagnetic elements 499 is 16 (13+3). In operation, this set up of magnetic gear changes the direction of rotation of the coupling, which means that that in operation the second coupling part 490b′ will rotate in the opposite direction and 4.33 times slower than the first coupling part 490a′. The embodiment having a magnetic gear have a number of advantages, for example, the magnetic gear is quiet, does not wear and does not need to be lubricated.

The second coupling part 490b′ is connected to a rotatable shaft which is supported by roller bearings 482 being fixated to the inside of the wall of the housing 484. The rotatable shaft comprises a threaded portion which is configured to be placed in and engage with a sleeve of a compression member 483. The sleeve of the compression member 483 comprises inside threads 483t for creating a transmission T that transforms the radially rotating force generated by the motor M and the gear system G, to a linear force acting in the axial direction of the shaft 481, and thus makes up a transmission T.

FIG. 137qq shows an embodiment of a hydraulic pump 104 which is similar to the embodiment shown in FIG. 137pp. The main difference with the embodiment shown in FIG. 137aq is that the expansion portion is replaced with two resilient reservoirs 107′, 107″ which are placed in indentations in the housing, on respective two opposite sides of the housing. The two resilient reservoirs 107′, 107″ are configured to expand and contract to compensate for the changes to the volume of the reservoir 107 which is created by the movement of the first resilient wall portion 102a of the reservoir 107. As such, the pressure in the second chamber C2 will be substantially constant. The two resilient reservoirs 107′, 107″ are made from a medical grade elastic silicone material but may in alternative embodiments be made from another biocompatible polymer material, such as polyurethane.

FIG. 137rr shows and embodiment of a system comprising a motor M, gear system G and two implantable pumps 460′, 460″. In the embodiment shown in FIG. 137rr, the force output of the motor M is connected to a force input of the gear system G. The gear system G is configured to reduce the velocity and increase the force of the movement generated by the motor M, such that the movement exiting the gear system G at the force output of the gear system G is a mechanical force with a lower velocity and a greater force than the movement entering the force input of the gear system G. Typically, an implantable brushless DC motor, such as the motors provided by Maxon group or Dr. Fritz Faulhaber, typically produces a rotational velocity exceeding 10,000 rpm. For such a motor to be able to mechanically operate any of the hydraulic pumps described herein, a gear system G is needed. In the embodiment shown with reference to FIG. 137rr, the gear system G reduces the rotational velocity 100 times, to about 100 rpm. The force output of the gear system G is mechanically connected to a common rotating shaft 463. The first hydraulic pump comprises a first gerotor pump 460′ and the second hydraulic pump comprises a second gerotor pump 460″. The common rotating shaft 463 is mechanically connected to an inner rotor 461′ of the first gerotor pump 460′ and an inner rotor 461″ of the second gerotor pump, such that the motor M propels the first and second gerotor pump 460′460″. A gerotor is a positive displacement pump comprising consists of an inner rotor 461 and an outer rotor 462. The inner rotor 461 has 6 teeth, while the outer rotor has 7 teeth (the importance being that the outer rotor 462 has one tooth more than the inner rotor 461. The axis of the inner rotor 461, which is the rotational center of the common rotating shaft 463, is offset from the rotational center or axis of the outer rotor 462. Both the inner and outer rotors 461, 462 rotate on their respective axes. The geometry of the two rotors 461, 462 partitions the volume between them into 6 different dynamically changing volumes. During the rotation cycle, each of these volumes changes continuously, so any given volume first increases, and then decreases. An increase creates a vacuum. This vacuum creates suction, and hence, this part of the cycle is where the inlet 109′ is located. As a volume decreases compression occurs which pumps the fluid though the outlet 109″.

In the embodiment shown in FIG. 137rr, the first gerotor pump 460′ is configured to be in fluid connection with a first operable hydraulic constriction element for pumping hydraulic fluid into the first operable hydraulic constriction element for inflating the first operable hydraulic constriction element to exert a pressure on the luminary organ and thereby restrict the flow or fluid therethrough. The second gerotor pump 460″ is configured to be in fluid connection with a second operable hydraulic constriction element for pumping hydraulic fluid into the second operable hydraulic constriction element for inflating the second operable hydraulic constriction element to exert a pressure on the luminary organ and thereby restrict the flow or fluid therethrough. The inlets 109′, 109″ of the first and second gerotor pumps 460′, 460″ are configured to be connected to a reservoir for holding hydraulic fluid, or in the alternative, the first inlet 109′ is configured to be connected to a first implantable reservoir and the second inlet 109″ is configured to be connected to a second implantable reservoir.

In alternative embodiments, the first and second hydraulic pump mechanically connected to a common rotating shaft could be pump comprising at least one compressible hydraulic reservoir (such as the pump described with reference to FIG. 137hh), a pump comprising a displaceable wall (such as the pump described with reference to FIG. 137gg), or a peristaltic pump.

With reference to FIGS. 137aaa-ccc, 138, 139, 100, and 141a-c, embodiments of an implantable energized medical device 100, which may be referred to as a remote unit in other parts of the present disclosure, will be described. As illustrated, these implantable energized medical devices have a second portion being shaped in a particular manner in order to facilitate removal of the implantable energized medical device once it has been implanted for a period of time and fibrotic tissue has begun to form around the second portion. It is hereby disclosed that these types of second portions, as illustrated in

FIGS. 137aaa-ccc, 138, 139, 100 and 141a-c, and as disclosed below, may be combined with any of the other features of the implantable energized medical device discussed in the present disclosure.

The device 100 is configured to be held in position by a tissue portion 610 of a patient. The device 100 comprises a first portion 141′ configured to be placed on a first side 612 of the tissue portion 610, the first portion 141′ having a first cross-sectional area in a first plane and comprising a first surface configured to face and/or engage a first tissue surface 616 of the first side 612 of the tissue portion 610.

The device 100 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface 622 of the second side 618 of the tissue portion 610. The device 100 further comprises a connecting portion 142 configured to be placed through a hole in the tissue portion 610 extending between the first and second sides 612, 618 of the tissue portion 610. The connecting portion 142 here has a third cross-sectional area in a third plane. The connecting portion 142 is configured to connect the first portion 141′ to the second portion 141″. In the illustrated embodiment, a connecting interface 630 between the connecting portion 142 and the second portion 141″ is arranged at an end of the second portion 141″.

The first portion 141′ may have an elongated shape. Similarly, the second portion 141″ may have an elongated shape. However, the first portion 141′ and/or second portion 141″ may assume other shapes, such as a flat disk e.g. having a width and length being larger than the height, a sphere, an ellipsoid, or any other polyhedral or irregular shape, some of these being exemplified in FIGS. 133-135.

To provide a frame of reference for the following disclosure, and as illustrated in FIGS. 138, 139 and 100, a first direction 631 is here parallel to the line A-A, to the second plane, and to a length of the second portion 141″. A second direction 633 is here parallel to the line B-B, to the second plane, and to a width of the second portion 141″. The second portion 141″ has a first end 632 and a second end 634 opposing the first end 632. The length of the second portion 141″ is defined as the length between the first end 632 and the second end 634. The length of the second portion 141″ is furthermore extending in a direction being different to the central extension C1 of the connecting portion 142. The first end 632 and second end 634 are separated in a direction parallel to the second plane. Similarly, the first portion 141′ has a length between a first and a second end, the length extending in a direction being different to the central extension C1 of the connecting portion 142.

The first portion 141′, connecting portion 142 and second portion 141″ may structurally form one integral unit. It is however also possible that the first portion 141′ and the connecting portion 142 structurally form one integral unit, while the second portion 141″ form a separate unit, or, that the second portion 141″ and the connecting portion 142 structurally form one integral unit, while the first portion 141′ form a separate unit.

Additionally, or alternatively, the second portion 141″ may comprise a removable and/or interchangeable portion 639 as described in other parts of the present disclosure.

In the following paragraphs, some features and properties of the second portion 141″ will be described. It is however to be understood that these features and properties may also apply to the first portion 141′.

The second portion 141″ has an intermediate region 638, and a distal region 640. A proximal region may be present, as described in other parts of the present disclosure, The intermediate region 638 is defined by the connecting interface 630 between the connecting portion 142 and the second portion 141″, and the distal region 640 extends from the connecting interface 630 between the connecting portion 142 and the second portion 141″ to the second end 634.

The first surface 614 configured to face and/or engage the first tissue surface 616 of the first side 612 of the tissue portion 610 may be substantially flat. In other words, the first portion 141′ may comprise a substantially flat side facing towards the tissue portion 610. Furthermore, an opposing surface of the first portion 141′, facing away from the tissue portion 610, may be substantially flat. Similarly, the second surface 620 configured to engage the second tissue surface 622 of the second side 618 of the tissue portion 610 may be substantially flat. In other words, the second portion 141″ may comprise a substantially flat side facing towards the tissue portion 610. Furthermore, an opposing surface of the second portion 141″, facing away from the tissue portion 610, may be substantially flat.

The second portion 141″ may be tapered from the first end 632 to the second end 634, thus giving the second portion 141″ different heights and/or widths along the length of the second portion 141″. The second portion may also be tapered from each of the first end 632 and second end 634 towards the intermediate region 638 of the second portion 141″.

Still referring to FIGS. 137aaa, 138, 139, 100, and 141a-c, the second portion 141″ and connecting portion 142 here form a connecting interface 630. Furthermore, the second portion 141″ has a lengthwise cross-sectional area along the first direction, wherein a second lengthwise cross-sectional area 690 is smaller than a first lengthwise cross-sectional area 689 and wherein the first lengthwise cross-sectional area 689 is located closer to the connecting interface 630 with regard to the first direction 631.

Hereby, a tapered second portion is formed, being tapered towards the second end 634. The lengthwise cross-sectional area of the second portion 141″ may decrease continuously from an end of the intermediate region 638 towards the second end 634, as illustrated for example in FIG. 138. The decrease may be linear, as illustrated for example in FIG. 138. However, other types of decreasing lengthwise cross-sectional areas are possible, such as a parabolic, exponential, stepwise, or stepwise with radiused edges between each step thus forming a smooth rounded contour.

FIGS. 137bbb and 137ccc illustrate how the lengthwise cross-sectional area decrease over the length of the second portion 141″ towards the second 634, as viewed along the line A-A. FIG. 137bbb illustrate the first lengthwise cross-sectional area 689, and FIG. 137ccc illustrate the second lengthwise cross-sectional area 690.

In some embodiments, the lengthwise cross-sectional area may decrease over a majority of the length of the second portion towards the second end 634. In some embodiments, a decrease of the lengthwise cross-sectional area over at least % of the length of the second portion towards the second end 634 may be sufficient. In the example illustrated in FIG. 138, the lengthwise cross-sectional area decrease over about 85% of the length of the second portion.

With the second portion 141″ having rotational symmetry along the first direction 631, as illustrated for example in FIG. 137aaa, the shape of the second portion 141″ may be conical.

As illustrated in FIG. 139, the second portion 141″ may have an upper surface, which include the second surface 620 configured to engage a second tissue surface of the second side of the tissue portion as discussed in other parts of the present disclosure, wherein the upper surface or second surface 620 is substantially flat and parallel to the second plane. In some embodiments the upper surface may be substantially perpendicular to the central extension C1 of the connecting portion 142. Hereby, the second surface may be configured to lay flat against the second side of the tissue portion. In such embodiments, a lower surface of the second portion 141″, opposite the second surface 620 and facing away from the first portion 141′, may be configured to taper towards the second end 634, thus achieving the decreasing lengthwise cross-sectional area along the first direction 631 towards the second end 634.

FIG. 100 illustrate an embodiment wherein the lengthwise cross-sectional area decrease in a stepwise manner towards the second end 634 of the second portion 141″. Here, the second portion 141″ has three major segments 692, 693, 694 having substantially constant diameter and each respective diameter being smaller moving towards the second end 634, being connected by intermediate segments 695, 696, wherein the diameter decreases along the first direction 631. Other variations of major segments having substantially constant diameter, and intermediate segments, having a decreasing diameter along the first direction 632, are possible, such as at least two major segments connected by a single intermediate segment with decreasing diameter, at least four major segments connected by three intermediate segments with decreasing diameter, and so on.

Referring now to FIGS. 141a-c, an implantable energized medical device similar to the one illustrated in FIG. 139 is illustrated. As can be seen in the perspective view of FIG. 141a, the second portion 141″ has a decreasing lengthwise cross-sectional area towards the second end. The upper surface 697 is also visible in this view, being substantially flat and providing a contact area to the second tissue surface 622. The first lengthwise cross-sectional area 689 is larger than the second cross-sectional area 690, as can be seen in FIGS. 141b-c, and the first lengthwise cross-sectional area 689 is located closer to the connecting interface between the connecting portion 142 and the second portion 141″ with regard to the first direction.

Referring now to FIG. 141d, an implantable energized medical device which may incorporate one or several of the features described in conjunction with FIGS. 125-141c, and which may be referred to as a remote unit in other parts of the present disclosure, will be described. The device 140 is configured to be held in position by a tissue portion 610 of a patient. The device 140 comprises a first portion 141′ configured to be placed on a first side 612 of the tissue portion 610, the first portion 141′ having a first cross-sectional area in a first plane and comprising a first surface configured to face and/or engage a first tissue surface of the first side 612 of the tissue portion 610. The device 140 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface of the second side 618 of the tissue portion 610. The device 140 further comprises a connecting portion 142 configured to be placed through a hole in the tissue portion 610 extending between the first and second sides 612, 618 of the tissue portion 610. The connecting portion 142 is configured to connect the first portion 141′ to the second portion 141″.

The connecting portion 142 and the second portion 141″ are configured to form a unit having a central axis C2 extending from a first end 650 of said unit to a second end 651 of said unit, the first end 650 being proximal to the first portion 141′ and the second end 651 being distal to the first portion 141′.

The first end 650 may generally be defined as the interface between the connecting portion 142 and the first portion 141′.

A physical footprint of the unit perpendicular to the central axis C2 decreases continuously or stepwise from the first end 650 to the second end 651 of said unit. Here, the physical footprint 652 is smaller than the physical footprint 653 which is more proximal to the first end 650, the physical footprint 653 in turn being smaller than the physical footprint 654 which is even more proximal to the first end 650. The illustrated footprints 652, 653, 654 may be cross-sectional areas which are determined in a plane perpendicular to the central axis C2. The footprints 652, 653, 654 may also be seen as the extension of the unit in a plane perpendicular to the central axis C2.

In embodiments where the unit comprises one or more bends or one or more angled sections, the physical footprint shall preferably decrease continuously or stepwise from the first end 650 to the second end 651 of the unit also along such bends or angled sections.

By decreasing the physical footprint along the central axis C2, removal of the device 140 may be facilitated. In particular, the device 10 may more easily slide out of scar tissue which has formed around the implanted device 140.

The connecting portion 142 and the second portion 141″ may be configured to reversibly connect to each other to form the unit. Such a connection may be a snap-fit connection, a magnetic type connection, a threaded connection, or a combination thereof, as described in other parts of the present disclosure. An irreversible connection between the connecting portion 142 and the second portion 141″ is also possible. In this sense, the term “irreversible” shall be understood as a connection which cannot be disengaged without damage or irreversible damage. It is also possible that the connecting portion 142 and the second portion 141″ are formed as a single body forming the unit. In such cases there are no seal or interface between the connecting portion 142 and the second portion 141″.

The unit here comprises an angled section forming a bend in the unit. The bend being about 900 as measured from the first end 650 to the second end 651. Hereby, a secure position is achieved, and a smaller vertical footprint, i.e. the space occupied by the device in a direction inwards to the center of the patient, may be achieved. The bend may be between 15° and 165°, such as between 30° and 150°, such as between 45° and 135°, such as substantially 90°.

Referring now to FIGS. 141e-141g, an implantable energized medical device which may incorporate one or several of the features described in conjunction with FIGS. 141d, and which may be referred to as a remote unit in other parts of the present disclosure, will be described.

The device 140 is configured to be held in position by a tissue portion 610 of a patient. The device 140 comprises a first portion 141′ configured to be placed on a first side 612 of the tissue portion 610, the first portion 141′ having a first cross-sectional area in a first plane and comprising a first surface configured to face and/or engage a first tissue surface of the first side 612 of the tissue portion 610. The device 140 further comprises a second portion 141″ configured to be placed on a second side 618 of the tissue portion 610, the second side 618 opposing the first side 612, the second portion 141″ having a second cross-sectional area in a second plane and comprising a second surface configured to engage a second tissue surface of the second side 618 of the tissue portion 610. The device 140 further comprises a connecting portion 142 configured to be placed through a hole in the tissue portion 610 extending between the first and second sides 612, 618 of the tissue portion 610. The connecting portion 142 is configured to connect the first portion 141′ to the second portion 141″.

The device 140 further comprises a hermetic seal arrangement, which may cover the first portion 141′, the connecting portion 142 and the second portion 141″, as visualized by hermetic seal arrangement 656a. Such hermetic seal arrangement may for example be achieved by a housing made of a metal, such as titanium. The hermetic seal arrangement may also cover only the connecting portion 142 and the second portion 141″, as visualized by hermetic seal arrangement 656b. Such hermetic seal arrangement may be achieved by the flexible structure 655 being sealed with regard to the first portion 141′, and further wherein the flexible structure 655 is either joined with the second portion 141″ in a sealing manner, or formed as an integral unit with the second portion 141″.

Any entry to the connecting portion 142 and/or the second portion 141″, may be achieved by means of a sealed entry (not shown). Such sealed entry can for example be achieved by a portion of an outside of connecting portion 142 and/or the second portion 141″ comprising a ceramic portion integrated in, or brazed to, the material of the connecting portion 142 and/or the second portion 141″ respectively. In such cases, the material of the connecting portion 142 and/or the second portion 141″ is preferably a metal, such as titanium. At least one metallic lead or conduit may travel through the sealed entry for transferring energy, information or fluid respectively from an outside of the device 140, also known as the wet side, to an inside of the device 140. The at least one metallic lead may in turn be integrated in, or brazed to, the ceramic portion. Thus, the at least one metallic lead can pass the sealed entry without being further insulated, such that the sealed entry can enable the transfer of electrical energy, information, or fluid, through a wall of titanium and ceramics, such that the connecting portion 142 and/or the second portion 141″ can be hermetically enclosed by the hermetical seal arrangement which reduces the risk of any fluid diffusing into the device 140, and in particular into the connecting portion 142 and/or the second portion 141″.

Here, the connecting portion 142 comprises a flexible structure 655 enabling the connecting portion 142 to flex. As can be seen in FIG. 141f, the connecting portion 142 may flex to accommodate for the thickness of the tissue portion 610, in this particular case the second portion 141″ is flexing downwards and away from the first portion 141′. However, the flexible structure 655 may be configured to allow flexing in any and all directions. In some embodiments, the flexible structure 655 may be configured to allow roll, pitch, and/or upwards and downwards movement with regard to the first portion 141′ and/or second portion 141″. As can be seen in FIG. 141g, the connecting portion 142 may flex to accommodate for the thickness of the tissue portion 610, in this particular case the first portion 141′ is flexing downwards and towards the second portion 141″.

The flexible structure 655 here comprises a bellows, which may be annularly fixated by means of soldering or welding to the first portion 141′ and/or the second portion 141″. The bellows may be a metallic bellows, and more specifically may be a titanium bellows. The flexible structure 655 may thus be flexible by means of elasticity of the metal or the titanium. Metals are generally dense which is advantageous as fluids do not easily diffuse through the metal. This reduces the risk that gas or fluid diffuses into the device 140.

The bellows of the flexible structure 655 may assume a relaxed state, i.e. where the structure is not biased. In such relaxed state the flexible structure 655 may have a length L1 as measured from the first portion 141′ to the second portion 141″. Once the flexible structure 655 is compressed, the length of the flexible structure 655 may decrease to a length L2. Conversely, if the flexible structure 655 is pulled, the length of the flexible structure 655 may increase to L3, being larger than both L1. Depending on the corrugated structure of the bellows, e.g. the dimensions of the corrugations and their frequency along the length of the bellows, different degrees of flexibility may be achieved.

The bellows comprise lowered portions and elevated portions. The lowered portions and elevated portions enable at least one of compression, expansion and flexing of the bellows. By compressing or expanding one side of the bellows, flexing of the first portion 141′ or second portion 141″ may be achieved.

If the bellows is made from a metal, the metal may be welded to form the corrugations of the bellows. Furthermore, the bellows, or the flexible structure 655, may form part of the hermetic seal arrangement.

The flexible structure 655 may have a substantially cylindrical shape, as illustrated in FIGS. 141e-141g. Such shape may provide for that flexing is available in all directions with little to no variation in resistance depending on the flexing direction.

Referring now to FIGS. 141h-141k, an implantable energized medical device similar to the one described in conjunction with FIGS. 141e-141g, and which may incorporate one or several of the features described in conjunction with FIGS. 125-141g, and which may be referred to as a remote unit in other parts of the present disclosure, will be described. In particular, it is shown how the device 140 may be inserted into a hole 611 in a tissue portion 610 of a patient.

Owing to the flexible structure 652 of the connecting portion 142, the first portion 141′ and second portion 141″ can be separated to increase the distance between respective ends 657, 659 of the first portion 141′ and second portion 141″. Hereby, the second portion 141″ can be inserted into the hole 611 in the tissue portion 610 without being hindered by the first portion 141′ abutting the tissue portion 610, as shown in FIG. 141i. Once a sufficiently large portion of the second portion 141″ has been inserted through the hole 611, the device 140 can be rotated to achieve the desired position in the tissue portion 610.

Referring now to FIGS. 141m, 141n and 141p-141s, an implantable energized medical device which may incorporate one or several of the features described in conjunction with FIGS. 125-141k, and which may be referred to as a remote unit in other parts of the present disclosure, will be described. It should be noted that FIGS. 141m, 141n and 141p-141s are schematic.

In each of the embodiments illustrated in FIGS. 141m-141s, the device 140 comprises a first portion 141′, a connecting portion 142, and a second portion 141″. The device 140 is placed in a hole in a tissue portion 610. The device 140 comprises an electric motor 661 wherein at least part of the electric motor is arranged within the connecting portion 142. The connecting portion 142 may be defined by an imaginary boundary 663 defined by the first surface 614 of the first portion 141′ extending through the connecting portion 142. Here, the imaginary boundary 663 lines up with the first side 612 of the tissue portion 610. The connecting portion 142 may be further defined by an imaginary boundary 667 defined by the second surface 620 of the second portion 141″ extending through the connecting portion 142. Here, the imaginary boundary 667 lines up with the second side 618 of the tissue portion 610.

Furthermore, in each of the illustrated embodiments of FIGS. 141m-141s, the electric motor 661 is preferably oriented such that its longest dimension aligns with a longest dimension of the device 140, in this case being a dimension extending from the first portion 141′ to the second portion 141″ as indicated by the reference sign LD.

Referring first to FIG. 141m, the electric motor 661 is fully arranged within the connecting portion 142.

In FIG. 141n, the electric motor 661 is arranged such that it extends through the connecting portion into the first portion 141′, past the imaginary boundary 663, and such that it extends through the connecting portion into the second portion 141″, past the imaginary boundary 667.

In FIG. 141p, the electric motor 661 is arranged such that it is contained within the imaginary boundary 663, in the connecting portion 142, and such that it extends through the connecting portion into the second portion 141″, past the imaginary boundary 667.

In FIG. 141q, a gear arrangement 669 is operatively connected to the electric motor 661. The gear arrangement may be located in the second portion 141″ or the connecting portion 142. The electric motor 661 may extend into the second portion 141″, past the imaginary boundary 667, as illustrated, or it may be located within the connecting portion 142 within the imaginary boundary 667. The gear arrangement 669 is configured to transfer mechanical force from the electric motor to an implantable body engaging portion (not shown) being external to the device 140, or to an actuating element in e.g. a pump (not shown). The gear arrangement 669 may be a worm drive, as illustrated. The gear arrangement 669 may further be configured to transfer movement from the electrical motor 661 to a different plane or axis than what is provided by the electric motor 661 itself. In the illustrated embodiment, the gear arrangement 669 transfers movement from the electrical motor 661 to a new axis being substantially perpendicular to the axis provided by the electric motor 661.

In FIG. 141r, an embodiment similar to the one described in conjunction with FIG. 141q is shown.

Here, a gear arrangement 669 is operatively connected to an electric motor 661. The electric motor 661 is arranged such that it is contained within the connecting portion 142, within the imaginary boundaries 663 and 667. However, the gear arrangement 669 is arranged in the second portion 141″.

In FIG. 141s, a gear arrangement 669 is operatively connected to the electric motor 661. The gear arrangement 669 as well as the electric motor 661 is arranged such that they are contained within the connecting portion 142, within the imaginary boundaries 663, 667.

Without reference to any particular figure, it is herein further disclosed that the implantable energized medical device 140 may comprise the first portion configured to be placed on a first side of the tissue portion, the first portion comprising a first surface configured to face a first tissue surface of the first side of the tissue portion, the first portion being further configured to connect, directly or indirectly, to a second portion, as disclosed in other parts of the present disclosure, placed on a second side of the tissue portion opposing the first side, wherein the first portion comprises an internal wireless energy transmitter configured to transmit energy wirelessly to the second portion. Such first portion may be configured to connect to the second portion via a connecting portion as disclosed in other parts of the present disclosure. The connecting portion may form part of such device 140, and may be integrally formed with the first portion, or may be a separate component with regard to the first portion, wherein the connecting portion is configured to connect to the first portion. In other words, one embodiment of the device 140 may consist of the first portion only, omitting the second portion and optionally omitting the connecting portion.

FIG. 141t shows a cross-sectional view of an implantable energized medical device, which may incorporate one or several of the features described in conjunction with FIGS. 1-141s, and which may be referred to as a remote unit in other parts of the present disclosure.

The second portion 141″ of the device 140 comprises or forms a reservoir 671 for holding a fluid, and device 140 further comprises a sealed container C configured to protrude into the reservoir 671. An actuator A is connected to the sealed container C, the actuator A being configured to expand or retract the sealed container C to change the volume of the sealed container C for pumping fluid to or from the reservoir 671.

The reservoir 671 is configured to hold the fluid to be pumped. The fluid is preferably a biocompatible incompressible liquid, such as a saline solution, but could in the alternative be an oil-based liquid, such as a silicone oil, or a gas. The sealed container C is configured to protrude into the reservoir 671, such that a wall of the sealed container forms a portion of a wall enclosing the reservoir 671. The sealed container C comprises a first movable wall portion 672 forming a portion of the wall of the reservoir 671. The movable wall portion 672 being a portion of the sealed container C protruding into the reservoir 671. The actuator A is directly or indirectly connected to the first movable wall portion 672, for moving the movable wall portion 672, for altering a volume of the sealed container C and a volume of the reservoir 671, for pumping the fluid to or from the reservoir 671.

The sealed container C may comprise a first portion (as shown) and a second portion (not shown), where movement of the first movable wall portion 672 causes movement of a second movable wall portion, altering a volume of the second portion of the sealed container C, such that the volume change of the sealed container C is less than the volume change of the reservoir 671, when the volume of the reservoir 671 is altered for pumping fluid to or from the reservoir 671. When the volume of the reservoir 671 is reduced and expanded, the reservoir 671 functions as a pump for moving fluid to and from a body engaging portion, i.e. an additional implant in the patient's body

The first and second portions of the sealed container may be mirrored and identical, and as such, in a relaxed state, have the same volume. The pressure in the sealed container remains substantially the same all the time as the volume of the sealed container C remains substantially the same when the volume of the reservoir 671 is altered for pumping fluid to or from the reservoir 671.

In the embodiment shown in FIG. 141t, the sealed container C is annular, and the center of the sealed container C is annularly fixated to outer portions of the reservoir 671. As the actuator moves the first movable wall portion 672 causing the sealed container C to expand, a second movable wall portion, if present, moves the same distance, contracting the second portion of the sealed container C, such that the volume in the sealed container remains the same when the volume of the reservoir 671 is altered for pumping fluid to or from the reservoir 671. The sealed container C is substantially rigid in directions other than the length extension LE of the sealed container C, such that the diameter of the sealed container is substantially constant and the volume of the sealed container only changes as a result of the first and second movable wall portions moving in the direction of the length extension of the sealed container C.

In the embodiment shown in FIG. 141t, the sealed container C comprises a titanium bellows, and the titanium bellows is annularly fixated by means of soldering or welding to the to reservoir or second portion 141″.

In the embodiment shown in FIG. 141t, the volume of the sealed container C is altered less than 10% when the volume of the reservoir is altered for pumping fluid to or from the reservoir 671. In particular, the volume of the sealed container C may be altered less than 5% when the volume of the reservoir 671 is altered for pumping fluid to or from the reservoir 671.

In the embodiment shown in FIG. 141t, the walls of the sealed container C comprises lowered portions and elevated portions. The lowered portions and elevated portions enable at least one of compression and expansion of the sealed container C. In particular, in the embodiment shown in FIG. 141t the sealed container C comprises a bellows enabling the contraction and expansion of the sealed container C, by means of the elasticity of the bellows making the bellows flexible. In particular, in the embodiment shown in FIG. 141t, the bellows is a metal bellows, in particular a titanium bellows. The sealed container C in the form of a titanium bellows is thus flexible by means of the elasticity of the titanium.

As the sealed container C is a titanium bellows 452, at least a portion of the first movable wall portion 672 being in contact with the fluid in the reservoir 671 comprises metal, namely the titanium. Metals are generally dense which is advantageous as fluids do not diffuse through the metal as easy. This reduces the risk that gas diffuses from the sealed container C or that fluids diffuse into the sealed container C. In the embodiment shown in FIG. 141t, the entire wall enclosing the sealed container C is made from metal, in particular titanium. In alternative embodiments it is however conceivable that a portion of the wall of the sealed container C is made from a flexible or elastic polymer material, such as a silicone-based material or a polyurethane-based material. In embodiments in which the sealed container has a wall made from composite of metallic or non-metallic materials, the non-metallic materials could be provided as a layer or a coating applied or sprayed onto the metal. In some embodiments, at least 50% of the area of the wall enclosing the sealed container C comprises metal and in alternative embodiments at least 80% of the area of the wall enclosing the sealed container C comprises metal, and in yet alternative embodiments, at least 90% of the area of the wall enclosing the sealed container C comprises metal.

The sealed container C may be configured to enclose a gas, such as helium or air. More specifically, the sealed container C may be configured to enclose a gas having a pressure exceeding standard atmospheric pressure (atm), i.e. 101,325 Pa. The sealed container C in the embodiment of FIG. 141t is hermetically enclosed by a metallic layer such that the gas enclosed in the sealed container C is hermetically enclosed by a metallic layer.

The device 140 may further comprise a fluid conduit 673 for connecting the reservoir 671 to a body engaging portion 676 of an implant configured for receiving the fluid pumped from the reservoir 671. The body engaging portion 676 is here an implantable constriction device in a state in which the implantable constriction device is constricting a luminary organ U and thereby restricts the flow of fluid through the luminary organ U. The implantable constriction device comprises a surrounding structure 677 having a periphery surrounding the luminary organ U when implanted. The surrounding structure 677 comprises two support elements 678a, 678b connected to each other for forming the surrounding structure 677. The first support element 678a is configured to support a first operable hydraulic constriction element 679. The first operable hydraulic constriction element 679 is configured to constrict the luminary organ U for restricting the flow of fluid therethrough and configured to release the constriction of the luminary organ U upon request. The first and second support elements 678a, 678b each comprises a curvature adapted for the curvature of the luminary organ U such that the implantable constriction device fits snuggly around the luminary organ U such that the distance that the operable hydraulic constriction elements 679 needs to expand to constrict the luminary organ U is kept at a minimum.

All foreign matter implanted into the human body inevitably causes an inflammatory response. In short, the process starts with the implanted medical device immediately and spontaneously acquiring a layer of host proteins. The blood protein-modified surface enables cells to attach to the surface enabling monocytes and macrophages to interact on the surface of the medical implant. The macrophages secrete proteins that modulate fibrosis and in turn developing the fibrosis capsule around the foreign body. In practice, a fibrosis capsule is a dense layer of excess fibrous connective tissue. On a medical device implanted in the abdomen, the fibrotic capsule typically grows to a thickness of about 0.5 mm-2 mm, and is substantially inelastic and dense.

Fluid is conducted from the reservoir 671 to the operable hydraulic constriction element 679 such that the implantable constriction device constricts the luminary organ U and thereby restricts a flow of fluid through the luminary organ U. As a first portion of the sealed container C is expanded and the second portion of the sealed container, if present, is compressed, the volume of the sealed container C remains the same throughout the shift, and as such, the actuator A does not need to work against a changing pressure in the sealed container C. The volume of the reservoir 671 when the sealed container C is in its most expanded state may be less than 50% of the volume of the reservoir 671 when the sealed container C is in its most compressed state.

The actuator A may comprise an electrical motor M configured to convert electrical energy to a rotating mechanical force. The motor M may be connected to a transmission configured to receive mechanical force and reduce the speed and increase the force of the received mechanical force. In the embodiment of FIG. 141t, the transmission is in the form of a gear arrangement G configured to increase the torque of the mechanical force created by the electrical motor M and to deliver a force with a higher torque. Consequently, low torque may be provided by the motor M, i.e. a relatively small force with high angular velocity, which is transferred to the gear arrangement G to achieve a relatively large force with low angular velocity. The gear arrangement is in turn connected to a further transmission configured to transform the received rotating mechanical force into a liner mechanical force. The further transmission comprises a shaft 674 connected to the force output of the gear arrangement G. The shaft comprises outer threads 674t adapted to engage inner threads of a nut portion 675 in the form of a hollow shaft having inner threads, such that the interaction between the threaded shaft 674, 674t and the threaded inner portion of the nut portion 675 transforms the radially rotating force generated by the motor M and the gear system G, to a linear force acting in the axial direction of the shaft 674. The nut portion 675 is fixated to, or integrated with, the first movable wall portion 672 of the sealed container C.

The sealed container C may further comprise a first and a second connecting member for connecting the first movable wall portion 672 to the second movable wall portion (not shown), such that the second movable wall portion moves in synchronization with the first movable wall portion 672, as the operation device operates the first movable wall portion 672. The first and second connecting members could be metal rods, such as titanium rods welded or soldered to the inner surfaces of the first and second movable wall portions respectively.

The actuator A is further arranged within the sealed container C, thus being protected from fluids in the reservoir 671 by means of the hermetic property of the sealed container C.

The reservoir 671 may have an oval cross-section, more specifically an elliptic cross-section, and even more specifically a circular cross-section. Having a circular cross-section enables the reservoir 671 to have the same cross-sectional shape as the sealed container C, which may also have an oval cross-section, more specifically an elliptic cross-section, and even more specifically a circular cross-section, which means that the distance between the wall of the bellows of the sealed container C can be made really small for reducing the space occupied by the device 140 in the body of the patient.

The sealed container C may further comprise a sealed entry in the form of a portion of the wall of the sealed container C comprising a ceramic portion integrated in, or brazed to, the metal or titanium of the sealed container C. At least one metallic lead may travel through the sealed entry for transferring electrical energy or information from within the sealed container C to the environment outside the sealed container C, also known as the wet side. The at least one metallic lead may in turn be integrated in, or brazed to, the ceramic portion. Thus, the at least one metallic lead can pass the sealed entry without being further insulated, such that the sealed entry can enable the transfer of electrical energy or information through a wall of titanium and ceramics, such that the sealed container C can be hermetically enclosed by titanium and ceramics which reduces the risk of any fluid diffusing into the sealed container C.

As shown in FIG. 141t, the device 140 may further comprise an injection port 681 configured to receive a needle or other means for transferring fluid to or from the device 140, in particular to or from the reservoir 671. The injection port 681 is connected to an internal conduit 682 which is fluidly connected to the reservoir 671 via a port 683. The injection port 681 may comprise a membrane configured to form a seal around the needle or other means for transferring fluid when penetrated.

Referring now to FIGS. 141u-141z and 141aa-141jj, embodiments of a system comprising an implantable energized medical device, which may incorporate one or several of the features described in conjunction with FIGS. 1-141t, and which may be referred to as a remote unit in other parts of the present disclosure.

Each system depicted in FIGS. 141u-141z and 141aa-141jj comprises an implantable energized medical device 140 configured to be held in position by a tissue portion 610 of a patient, the device 140 comprising: a first portion 141′, a second portion 141″, and a connecting portion 142, as described elsewhere in the present disclosure. The system may further comprise at least one of the following components: an implantable energy storage unit 304, an implantable reservoir 107 configured to hold a fluid, a body engaging implant 676 configured to at least one of stretch, contract, expand, stimulate and exert a force on body tissue or a body organ, an implantable pump 104 configured to transfer fluid to and from the implantable reservoir and the body engaging implant respectively via a conduit, and an implantable electric motor 661 connected to the implantable energy storage unit 304, the implantable electric motor 661 being configured to operate the implantable pump 104. The embodiments differ mainly in the arrangement and location of the components, in particular whether the components are arranged within the device, i.e. in the first portion 141′, connecting portion 142 and/or second portion 141″, or whether they are arranged external to the device, i.e. outside of the first portion 141′, connecting portion 142 and/or second portion 141″.

As illustrated, the implantable pump 104 may be connected to the implantable reservoir 107 via a fluid conduit.

The expression “arranged externally” shall in this context be understood as a component being configured to be located outside of the device 140. Furthermore, such component may be configured to be implanted in a location in the patient's body which is remote to the location of the implanted device 140.

Furthermore, each system comprises an internal component 685 arranged in the device 140, wherein the internal component 685 may have capabilities of at least one of receiving wireless energy, transmitting wireless energy, receiving communication signals, and transmitting communication signals. The internal component 685, although here illustrated as a single unit, may comprise several units.

The illustrated embodiments are purely schematic, and lines connecting components may symbolize one or several metallic leads for transferring energy and/or for transferring communication signals (such may be the case for lines between the implantable energy storage unit 304 and the implantable electric motor 661). They may also symbolize a shaft or magnetic coupling configured to transfer force or movement (such may be the case for lines between the implantable electric motor 661 and the implantable pump 104). It shall also be noted that the lines connecting components does not necessarily imply that the components are physical separated and connected by e.g. metallic leads or shafts. They may instead simply be seen as an indication that the components are connected electrically or mechanically. For example, in the case of the implantable electric motor 661 and the implantable pump 104, these two components, although illustrated as being separate and connected by a line, may form part of a single unit which integrates the implantable electric motor 661 with the implantable pump 104 to achieve the functionality of a pump. Furthermore, the implantable pump 104 may form part of the implantable reservoir 104, and in particular the implantable pump 104 may be arranged at least partly inside or in connection with the implantable reservoir 104.

Furthermore, the location of the components in relation to the device 140, internally and externally, as well as the length of the illustrated lines and conduits, shall not be seen as limited by the illustrated embodiments. In contrast, for example, the length of lines and conduits may be shorter or longer than depicted. Similarly, illustrated components may be located closer to or further away from each other, and/or closer to or further away from the device 140, and/or in other locations or portions of the device 140. Furthermore, the entrance and exit of lines or conduits with regard to the device 140 are schematic and only exemplary.

Furthermore, although not shown in the illustrated embodiments, a conduit in fluid connection with the reservoir may be arranged internally and/or externally to the device 140 for introducing and/or removing fluid from the reservoir. The conduit may be connected to an injection port in the device 140.

A gear arrangement may be included in the system, preferably arranged in proximity of the electric motor 661. The gear arrangement, if present, is operatively connected to the electric motor and configured to reduce the velocity and increase the force of movement generated by the electric motor 661. Thus, if the electric motor 661 is arranged externally with the regard to the device 140, the gear arrangement may be arranged externally as well, or internally, i.e. within the device 140, and vice versa.

An advantage of having one or more components externally arranged to the device 140 is that load distribution may be improved. This is particularly important since one of the objects of the device 140 is to achieve and maintain a secure placement in the patient's body. By distributing weight to other parts of the patient's body, the risk of detachment of the device 140 from its implanted position may be decreased. Furthermore, it may be advantageous to distribute heat generation from one or more of the components to particular parts or regions or several parts or regions of the patient's body.

Referring first to FIG. 141u, the system comprises, externally arranged with regard to the device 140, an implantable energy storage unit 304, an implantable electric motor 661, an implantable reservoir 107, and an implantable pump 104.

Referring now to FIG. 141v, the system comprises, externally arranged with regard to the device 140, an implantable electric motor 661, an implantable reservoir 107, and an implantable pump 104.

Furthermore, the system comprises an implantable energy storage unit 304 arranged in the device 140.

Referring now to FIG. 141w, the system comprises, externally arranged with regard to the device 140, an implantable energy storage unit 304, an implantable reservoir 107, and an implantable pump 104.

Furthermore, the system comprises an implantable electric motor 661 arranged in the device 140.

Referring now to FIG. 141x, the system comprises, externally arranged with regard to the device 140, an implantable energy storage unit 304, an implantable electric motor 661, and an implantable pump 104. Furthermore, the system comprises an implantable reservoir 107 arranged in the device 140.

Referring now to FIG. 141y, the system comprises, externally arranged with regard to the device 140, an implantable energy storage unit 304, an implantable electric motor 661, and an implantable reservoir 107. Furthermore, the system comprises an implantable pump 104 arranged in the device 104.

Referring now to FIG. 141z, the system comprises, externally arranged with regard to the device 140, an implantable energy storage unit 304 and an implantable electric motor 661. It should be noted that this embodiment does not necessarily comprise an implantable reservoir 107 nor an implantable pump 104. The implantable electric motor 661 is here operatively connected to the body engaging implant 676.

Referring now to FIG. 141aa, the system comprises, externally arranged with regard to the device 140, an implantable reservoir 107 and an implantable pump 104. Furthermore, the system comprises an implantable energy storage unit 304 and an implantable electric motor 661, arranged in the device 104.

Referring now to FIG. 141bb, the system comprises, externally arranged with regard to the device 140, an implantable energy storage unit 304 and an implantable pump 104. Furthermore, the system comprises an implantable electric motor 661 and an implantable reservoir 107, arranged in the device 140.

Referring now to FIG. 141ce, the system comprises, externally arranged with regard to the device 140, an implantable energy storage unit 304 and an implantable electric motor 661. Furthermore, the system comprises an implantable reservoir 107 and an implantable pump 104, arranged in the device 140.

Referring now to FIG. 141dd, the system comprises, externally arranged with regard to the device 140, an implantable electric motor 661 and an implantable reservoir 107. Furthermore, the system comprises an implantable energy storage unit 304 and an implantable pump 104, arranged in the device 104.

Referring now to FIG. 141ee, the system comprises, externally arranged with regard to the device 140, an implantable energy storage unit 304. Furthermore, the system comprises an implantable electric motor 661 arranged in the device 104. It should be noted that this embodiment does not necessarily comprise an implantable reservoir 107 nor an implantable pump 104. The implantable electric motor 661 is here operatively connected to the body engaging implant 676.

Referring now to FIG. 141ff, the system comprises, externally arranged with regard to the device 140, an implantable pump 104. The system further comprises an implantable energy storage unit 304, an implantable electric motor 661, and an implantable reservoir 107, arranged in the device 104.

Referring now to FIG. 141gg, the system comprises, externally arranged with regard to the device 140, an implantable energy storage unit 304. The system further comprises an implantable pump 104, an implantable electric motor 661, and an implantable reservoir 107, arranged in the device 104.

Referring now to FIG. 141hh, the system comprises, externally arranged with regard to the device 140, an implantable electric motor 661. The system further comprises an implantable energy storage unit 304, an implantable pump 104, and an implantable reservoir 107, arranged in the device 104.

Referring now to FIG. 141ii, the system comprises, externally arranged with regard to the device 140, an implantable reservoir 107. The system further comprises an implantable energy storage unit 304, an implantable electric motor 661, an implantable pump 104, arranged in the device 104.

Referring now to FIG. 141jj, the system comprises, externally arranged with regard to the device 140, and an implantable electric motor 661. Furthermore, the system comprises an implantable energy storage unit 304 arranged in the device 104. It should be noted that this embodiment does not necessarily comprise an implantable reservoir 107 nor an implantable pump 104. The implantable electric motor 661 is here operatively connected to the body engaging implant 676.

Referring now to FIG. 141kk, an implantable energized medical device which may incorporate one or several of the features described in conjunction with FIGS. 1-141jj, and which may be referred to as a remote unit in other parts of the present disclosure, will be described. The device 140 is configured to be held in position by a tissue portion of a patient. The device 140 comprises a first portion 141′ configured to be placed on a first side of the tissue portion, the first portion 141′ having a first cross-sectional area in a first plane and comprising a first surface configured to face and/or engage a first tissue surface of the first side of the tissue portion. The device 140 further comprises a second portion 141″ configured to be placed on a second side of the tissue portion, the second side opposing the first side, the second portion 141″ having a second cross-sectional area in a second plane and comprising a second surface configured to engage a second tissue surface of the second side of the tissue portion. The device 140 further comprises a connecting portion 142 configured to be placed through a hole in the tissue portion extending between the first and second sides of the tissue portion. The connecting portion 142 is configured to connect the first portion 141′ to the second portion 141″.

The device 140 here comprises an internal component 685 arranged in the device 140, wherein the internal component 685 may have capabilities of at least one of receiving wireless energy, transmitting wireless energy, receiving communication signals, and transmitting communication signals.

The internal component 685, although here illustrated as a single unit, may comprise several units.

The second portion 141″ is here hermetically sealed by means of an outer wall 686 of the second portion comprising a metal, such as titanium. The second portion 141″ may further comprise an internal component 684 having capabilities of at least one of receiving wireless energy, receiving wired energy, receiving communication signals, and transmitting communication signals. Such internal component 684 may comprise an electric motor, a pump, or the like.

An outer wall of the first portion 141′ may comprise or consist of a polymer material. Accordingly, fluid will likely be able to permeate through the outer wall of the first portion 141′ over time when the device 140 is implanted. In order to protect components of the second portion 141″, a hermetic seal is formed with respect to the connecting portion 142 and with respect to the first portion 141′.

Furthermore, in order to achieve wired communication or energy transfer between the second portion 141″ and the connecting portion 142, the outer wall 686 of the second portion 141″ may comprise a ceramic portion 687 integrated in, or brazed to, the outer wall 686. The ceramic portion 687 may in turn comprise at least one metallic lead 691 travelling through the ceramic portion 687 for transferring electrical energy or information from within the second portion 141″ to an outside of the second portion 141″ and/or from the outside of the second portion 141″, such as from the connecting portion 142, to an inside of the second portion 141″. The outside of the second portion 141″, being outside of the hermetic seal, is commonly referred to as the “wet” side. The at least one metallic lead 691 may in turn be integrated in, or brazed to, the ceramic portion 687, such that the at least one metallic lead 687 can pass the ceramic portion 687 without being further insulated.

Similarly, the connecting portion 142 may comprise an outer wall comprising a metal, such as titanium. Such outer wall of the connecting portion 142 may form a hermetic seal. Furthermore, the outer wall of the connecting portion 142 may comprise a ceramic portion integrated in, or brazed to, the titanium (not shown). At least one metallic lead may travel through the ceramic portion for transferring electrical energy or information from within the connecting portion to an outside of the connecting portion and/or from the outside of the connecting portion to an inside of the connecting portion. The least one metallic lead may be integrated in, or brazed to, the ceramic portion of the connecting portion, such that the at least one metallic lead can pass said ceramic portion without being further insulated.

The at least one metallic lead 691 may connect, or extend to form, an internal lead 698. Such internal lead 698 may be connected to the internal component 684, as illustrated.

Accordingly, when the connecting portion 142 and the second portion 141″ engage, the at least one metallic lead 691 will engage with a corresponding metallic lead 647 of the connecting portion to form a connection for transferring wired energy and/or wired communication signals. Owing to the integrated ceramic portion 687, the transfer of such wired energy and/or wired communication signals can be achieved through the boundary of the second portion 141″ without breaking the hermetic seal.

Referring now to FIG. 141mm, a device 140 similar to the one described in conjunction with FIG. 141kk is illustrated. However, here, no ceramic port is necessary since the internal component 685 located in the first portion 141′ and the internal component 684 located in the second portion 141″ are configured to transmit and/or receive wireless energy and/or wireless communication signals. Thus, the outer wall 686 of the second portion 141″, forming a hermetic seal, need not be penetrated.

Referring now to FIGS. 141nn and 141pp, an implantable energized medical device which may incorporate one or several of the features described in conjunction with FIGS. 1-141mm, and which may be referred to as a remote unit in other parts of the present disclosure, will be described.

The device 140 is configured to be held in position by a tissue portion 610 of a patient. The device 140 comprises a first portion 141′ configured to be placed on a first side of the tissue portion 610, the first portion 141′ having a first cross-sectional area in a first plane and comprising a first surface configured to face and/or engage a first tissue surface of the first side of the tissue portion 610. The device 140 further comprises a second portion 141″ configured to be placed on a second side of the tissue portion 610, the second side opposing the first side, the second portion 141″ having a second cross-sectional area in a second plane and comprising a second surface configured to engage a second tissue surface of the second side of the tissue portion 610. The device 140 further comprises a connecting portion 142 configured to be placed through a hole in the tissue portion 610 extending between the first and second sides of the tissue portion. The connecting portion 142 is configured to connect the first portion 141′ to the second portion 141″.

As illustrated in FIG. 141nn, the first portion 141′ is configured to be placed subcutaneously, which is also evident from its implanted position relative to the surface of the skin 699 of the patient.

Furthermore, the first portion may comprise a connecting interface arrangement 641 configured to transfer wired energy and/or wired communication signals and/or fluid to an additional implant in the patient. The connecting interface arrangement 641 is here illustrated as a single unit, it is however to be understood that the connecting interface arrangement 641 may include one or several connecting interface units at different locations on the device 140. In particular, a connecting interface for fluid may require a separate connecting interface unit, or port, and a connecting interface for wired energy or communication signals may require another separate connecting interface unit.

A lead, wire or fluid conduit 643 may form part of the device 140. Such components may also form part of a system which includes the device 140. The lead or wire 643 is configured to connect to the connecting interface arrangement 641 for transferring wired energy and/or wired communication signals. Similarly, the fluid conduit 643 is configured to connect to the connecting interface arrangement 641 for transferring fluid to and from the device 140 and a body engaging implant implanted in another part of the patient's body (not shown), and/or a reservoir implanted in another part of the patient's body (not shown), and/or a pump implanted in another part of the patient's body (not shown).

By flipping the device 140 so that the first portion 140′ and the second portion 141″ switch places, i.e. so that the second portion 141″ is instead located closest to the surface of the skin 699, a wire, lead and/or fluid conduit 643 may run below the tissue portion 610 as opposed to above the tissue portion 610. Whether to run a lead, wire or fluid conduit below the tissue portion 610 or above the tissue portion 610 may be chosen depending on where the lead, wire or fluid conduit shall connect to an additional implant, where such additional implant is located, and/or how such additional implant is implanted. Thus, both the first portion 141′ and the second portion 141″ may be configured to be placed subcutaneously, i.e. closest to the surface of the skin 699 relative to the tissue portion 610, such that the device 140 can be placed with either of the first portion 141′ and the second portion 141″ on side of the tissue portion 610 being closest to the surface of the skin 699.

The connecting interface arrangement 641 may alternatively or additionally be arranged at the second portion 141″, as shown in FIG. 141pp. Thus, a lead, wire or fluid conduit may run above the tissue portion 610 even when the first portion 141′ is implanted below the tissue portion 610. Similarly, by flipping the device 140 as shown in FIG. 141pp, the first portion 141′ will be located above the tissue portion 610 and closest to the surface of the skin 699, and the second portion 141″ will be located below the tissue portion. Thus, a lead, wire or fluid conduit may run below the tissue portion 610 in this configuration.

The terms “above” and “below” in this context shall be understood as directional references where closer to the surface of the skin is “higher” or “above”, and further towards the center of the patient is “lower” or “below”.

A height H1 of the first portion 141′ may be 15 mm or less, such as 10 mm or less, such as 7 mm or less, such as 5 mm or less. The height H1 may be a maximum height of the first portion 141′, i.e. the height H1 may be defined as the height at the location where the first portion 141′ has the largest height. Likewise, a height H2 of the second portion 141″ may be 15 mm or less, such as 10 mm or less, such as 7 mm or less, such as 5 mm or less. The height H2 may be a maximum height of the second portion 141″, i.e. the height H2 may be defined as the height at the location where the first portion 141″ has the largest height.

A length L1 of the first portion 141′ and a length L2 of the second portion 141″ may differ no more than 30%, such as no more than 15%, such as no more than 5%, such as no more than 1%, such as wherein the length L1 of the first portion 141′ and the length L2 of the second portion 141″ are substantially equal, as illustrated.

Similarly, a width (not shown, measured in a direction extending inwards or outwards of the illustrated plane) W1 of the first portion 141′ and a width W2 of the second portion 141″ may differ no more than 30%, such as no more than 15%, such as no more than 5%, such as no more than 1%, such as wherein the width W1 of the first portion 141′ and the width W2 of the second portion 141″ are substantially equal.

Similarly, a height H1 of the first portion 141′ and a height H2 of the second portion 141″ may differ no more than 30%, such as no more than 15%, such as no more than 5%, such as no more than 1%, such as wherein the height H1 of the first portion 141′ and the height H2 of the second portion 141″ are substantially equal, as illustrated.

### B: Artificial Intestine Sections

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating artificial intestine sections, examples of which will now be described.

FIG. 142 shows a system with an implantable medical device 100 in the form of artificial intestine section being implanted inside a patient's body and having a first open end portion connected to a surgically created opening in the patient's intestine, more specifically to a lateral opening in a wall of the patient's intestine. The second open end portion exits the patient's abdominal wall forming a stomy. The artificial intestine section is here shown as a black box and includes at least one energy consuming part, such as one or more valves, a pump and/or any other flow control device, a motor for driving the same, possibly in connection with a reservoir. An accumulator is implanted along with the artificial intestine section and can be wirelessly charged from outside the patient's body. The energy is here galvanically transmitted from the accumulator to the artificial intestine section.

FIG. 143 shows a system corresponding to the one shown in FIG. 142, however, with the energy being transmitted wirelessly from the accumulator to the artificial intestine section.

FIG. 144 shows a system corresponding to the one shown in FIG. 142, however, with the second open end portion of the artificial intestine section exiting the patient's anus.

FIG. 145 shows a system where both the first and second open end portions of the artificial intestine section are attached to surgically created lateral openings in a wall of the patient's small and/or large intestine. The downstream part of the intestine exits the patient's abdominal wall forming a surgically created stomy. The downstream part of the intestine may as well exit through the patient's anus.

FIG. 146 shows a similar system with the difference that the second open end portion is connected to a cross-sectional opening of the patient's intestine, further leading to the surgically created stomy. The downstream part of the intestine may as well exit through the patient's anus.

FIG. 147 shows an embodiment of the artificial intestine section with an artificial reservoir and an entry valve and exit valve arranged upstream and downstream of the reservoir. The reservoir is mounted with a pump in a common housing and the pump and the entry and exit valves are controlled by means of a control device 102, 200, of which a part 102 is implanted inside the patient's body. Data are transmitted wirelessly between the external part 200 and implanted part 102 of the control unit. In addition, energy is wirelessly transmitted to the artificial intestine section or to an accumulator also implanted in the patient's body and galvanically connected here to the valves and pump.

FIGS. 148A and 148B show a first embodiment of the structure of FIG. 147 in more detail. The pump comprises a moveable piston with a front end of the piston extending into the reservoir such that a volume of the reservoir is reduced upon advancement of the piston. The piston is spring loaded so as to urge the piston into a normally retracted position. Furthermore, entry and exit valves are here realized as flap valves. The flap valves are controlled so that one valve is open while the other one is closed.

FIGS. 149B and 149B show a system similar to the one of FIGS. 148A and 148B. However, here the entry and exit valves comprise bellows acting on the intestine from the outside so as to close the intestine by compression. In FIG. 149A the bellows of the exit valve are expanded to compress the artificial intestine section at the downstream side of the reservoir, whereas in FIG. 149B the artificial intestine section is closed by means of the bellows of the entry valve upstream of the reservoir so that the reservoir can be emptied by advancing the piston of the pump.

FIG. 150 shows an embodiment schematically, wherein the artificial intestine section by-passes a section of the patient's intestine, the intestine being closed by sewing so as to direct intestinal content towards the artificial intestine section. The enlarged area of the artificial intestine section represents any kind of element acting on the intestinal contents within the artificial intestine section, such as a reservoir, one or more valves, a pump or any other flow control device, possibly including a motor, and the like.

Furthermore, a battery implantable in the patient's body and preferably rechargeable provides the artificial intestine section with energy. The artificial intestine section is wirelessly controlled and the battery, if rechargeable, wirelessly charged. A sensor implanted on or within the intestine delivers data on the physical conditions within the intestine for controlling the artificial intestine section.

FIGS. 151A to 151C show an embodiment, where the artificial intestine section comprises a reservoir with a flexible wall. A pump is implanted in the patient's body separate but in close proximity to the reservoir and is used to empty the reservoir. The pump is actuated by means of a subcutaneously implanted, manually operable switch.

FIGS. 152A and 152B show a structure similar to the one of FIGS. 151A to 151C, however, with the pump and the reservoir being fixedly connected to one another. The reservoir is formed by a bellow having an end wall closing the bellow at one end thereof. The end wall makes part of the pump such that a volume of the bellow can be reduced upon advancement of the end wall. The bellow is made of a resilient material so as to urge the bellow into a normally extended position.

FIGS. 153A to 153C show a plurality of cooperating valves implanted inside the patient's body and outside the patient's intestine. These can be positioned behind and/or in front of the artificial intestine piece along the patient's natural intestine. Each of the valves comprises an electrical stimulation device adapted to electrically stimulate muscle or neural tissue of an intestine section so as to cause at least partial contraction of the intestine section. For that purpose, the stimulation device comprises at least one electrode adapted to apply electric pulses to the intestine section. While instead of the three stimulation devices shown, a single stimulation device would be sufficient for opening and closing the intestine, the arrangement of the plurality of stimulation devices is adapted to stimulate different portions of the intestine section over time. The function of the three stimulation devices may also be combined in one integral unit. The direction of natural intestinal contents flow is indicated by arrows. The different portions of the intestine section in a wavelike manner may be made in a direction opposite to the natural intestinal contents flow, as shown in FIGS. 153A to 153C, so as to close the intestine section. The stimulation in the wavelike manner may also be made in the direction of natural intestinal contents flow to support emptying of the intestine or reservoir.

FIGS. 154A to 154C show the stimulation devices of FIGS. 153A—153C in combination with constriction devices, such as the bellow valves described in relation to FIGS. 149A and 149B, for at least partly constricting the intestine section mechanically. Complete constriction is obtained by additional electrical stimulation of the respective intestine sections. The constriction devices may be released in order to allow intestinal contents to flow through.

FIGS. 155A and 155B show a system comprising the artificial intestine section connected to a cross-sectional opening of the patient's intestine and having a valve as shown in FIG. 153 or 154 arranged around the patient's intestine upstream of the artificial intestine section. Energy and/or data is transmitted wirelessly.

FIG. 156 shows the structure of an open end portion of the artificial intestine section for attaching the artificial intestine section to a lateral opening in the patient's intestine by means of a shoulder portion formed around the end portion. The end portion is sewn to the intestine and may additionally or alternatively be stapled and/or glued to the intestine.

FIG. 157 shows an improved structure for lateral attachment to the intestine, wherein the shoulder portion is split into an upper and a lower shoulder portion forming a gap to accommodate intestinal wall tissue therein. The surface area of the upper shoulder portion is larger than the surface area of the lower shoulder portion.

FIG. 158 shows an enlarged view of a ring-and-bulge connection by which the artificial intestine section and the patient's downstream intestinal part are connected, as shown in FIG. 146.

FIGS. 159A and 159B show the ring-and-bulge connection of FIG. 158 in combination with a sleeve. The sleeve is rolled upon itself and can be unrolled such that a part of the intestine is located intermediate the sleeve and the conduit. Thereafter, the ring is pushed over the sleeve against the bulge.

FIGS. 160A and 160B show a connection of the artificial intestine section to a cross-sectional opening of the patient's intestine similar to the connection shown in FIGS. 159A and 159B, however, without the bulge and the ring.

FIGS. 161A and 161B show an alternative to the connection in FIGS. 160A and 160B. Instead of unrolling the sleeve, it is simply pulled over the intestine.

FIGS. 162A and 162B show another sleeve connection. Here, the sleeve is mounted on the outer surface of the open end portion so as to be foldable upon itself. By folding the flexible sleeve upon itself, a part of the intestine is located intermediate the folded sleeve.

### C: Intestinal Reservoir with Implantable Flow Control Device

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating implantable flow control devices which control the flow of intestinal contents in relation to an intestinal reservoir, examples of which will now be described.

FIG. 163A shows an implantable medical device 100 comprising a reservoir 140 formed from human intestine 70. A plurality of bent portions of the human's intestine 70 is cut open along the mutual contact lines of laterally adjacent sections thereof. The resulting upper halves and lower halves are interconnected so as to form the walls of the intestinal reservoir 140. The interconnection can advantageously be made with staplers, possibly including bonding with a biocompatible glue, but sewing is likewise an option.

At the exit of the intestinal reservoir 140, an exit valve comprising a plurality of valve sections 61, 62, 63 is provided along and encloses a non-modified terminate section 80 of the patient's intestine. The non-modified terminate section 80 is passed through the patient's abdominal wall AW and forms a surgically created stoma 170. The non-modified terminate section 80 could like-wise lead to the patient's rectum or anus. The valve sections 61, 62, 63 each comprise an electrical stimulation device adapted to electrically stimulate muscle or neutral tissue of the intestine's terminate section 80 so as to cause at least partial contraction of the terminate section. Electrical stimulation is achieved by applying electrical pulses to the terminate section 80 by means of electrodes (not shown). Each of the valve sections 61, 62, 63 further comprises at least one constriction device.

In FIG. 163A the constriction devices of all three valve sections 61, 62, 63 are activated. As can be seen, the constriction devices 61, 62, 63 only partly constrict the intestine's terminate section 80 so that blood circulation in the tissue of the intestinal wall is not negatively affected thereby. The electrical stimulation devices of the valve sections 61, 62, 63 are adapted to further constrict the terminate section 80 so that flow through the terminate section 80 is completely prevented. However, only one electrical stimulation device is activated at a time. In the situation shown in FIG. 163A, the central valve section 62 is currently activated so as to stimulate and thereby completely constrict the corresponding section of the intestine's terminate section 80. While instead of the three stimulation devices shown, a single stimulation device would be sufficient for opening and closing the intestine, the arrangement of the plurality of stimulation devices allows to stimulate different sections of the intestine's terminate section 80 over time. The function of the three stimulation devices may also be combined in one integral unit.

Since the electrical stimulation in each valve section 61, 62, 63 always occurs for a short time period only, the respective other, non-stimulated sections of the intestine's terminate section 80 have time to recover from a previous constriction so that sufficient blood flow within the intestinal wall is ensured.

All in all, the valve sections 61, 62, 63 allow for gentle constriction of the intestine's terminate section 80 at the exit of the reservoir when keeping the exit normally closed. Most preferably, closing is achieved by stimulating different sections of the intestine's terminate section 80 in a wave-like manner in a direction opposite to the natural intestine contents flow.

However, instead of combining electrical stimulation devices with a constriction device, the valve at the exit of the reservoir 140 may only be formed by one or a plurality of constriction devices. The constriction device is preferably of the hydraulic type, such as in the form of pressure cuffs, but may also be of the mechanical type. The constriction device is not described here in more detail, and may correspond to the entry valve 194 provided at the entry of the reservoir 140. The entry valve 194 here has the form of a hydraulic cuff. While the valve sections 61, 62, 63 of the exit valve are provided to normally close the exit of the intestinal reservoir 140 in order to keep intestinal contents inside the reservoir 140, the entry valve 194 is normally open to allow intestinal contents to flow into the reservoir 140.

The cuff of the entry valve 194 can be filled with a hydraulic fluid from an artificial hydraulic reservoir 195 so as to completely constrict the intestine 70 in front of the reservoir 140. This way, backflow of intestinal contents into the intestine 70 may effectively be prevented, when emptying of the reservoir is desired. At the same time, the valve sections 61, 62, 63 of the exit valve are opened to allow emptying of the intestinal reservoir 140. This is shown in FIG. 163B. As can be seen, the partial constriction of the terminal section 80 by means of the constriction devices has been released. Also, electrical stimulation pulses are no longer applied. However, it can be advantageous to support the emptying process by constricting the different sections of the intestine's terminate section 80 in a wave-like manner in a direction towards the stoma 170 by means of the valve sections 61, 62, 63 of the exit valve.

In the following, different embodiments of an implantable medical device 100 in the form of a system for emptying the intestinal reservoir 140 are described.

As shown in FIG. 164A, the reservoir 140 may be emptied by means of an electrical stimulation type pump comprising electrical stimulation devices 160 which are adapted to electrically stimulate muscle or neural tissue of the intestinal reservoir 140 so as to cause at least partial contraction of the reservoir 140. This is a very gentle way of constricting the tissue of the intestinal reservoir 140. A second set of electrical stimulation devices 161 is arranged on the opposite side of the reservoir 140, as can be seen in FIG. 164B. Thus, the stimulation devices 160, 161 are arranged substantially in two planes at opposite sides of the reservoir 140. The stimulation devices 160, 161 have a longitudinal shape so as to span over the reservoir 140 when arranged side by side as shown in FIG. 164A.

As shown in FIG. 164A, the stimulation devices 160, 161 each have a longitudinal or rod-like shape substantially spanning the entire width of the reservoir 140. The length is 10 cm or longer, depending on the size of the intestinal reservoir. The overall spanned area would typically be larger than 10 cm×10 cm in plan view. The stimulation devices 160, 161 may each comprise a row of electrodes arranged along the length thereof and adapted to apply electric pulses to the intestinal wall of the reservoir 140. Alternatively, each stimulation device may substantially consist of only one longitudinal electrode. Preferably, the electrodes can be controlled individually.

In another embodiment, not shown, the stimulation devices 160, 161 may form plate-like members having a larger width than those shown in FIG. 164A, resulting in a decreased number of stimulation devices. In an even further embodiment, likewise not shown, instead of arranging the stimulation devices separately side by side, they may be combined in an integral unit, such as a plate, on one side or on opposing sides of the intestinal reservoir. In case that the stimulation device or devices form plate-like members with an enlarged width, a plurality of electrode rows may be arranged in parallel on the stimulation devices. The plate-like or rod-like stimulation devices may be embedded in a flexible web (not shown) to facilitate implantation and relative fixation of adjacent stimulation devices.

Emptying of the intestinal reservoir 140 can be activated by the patient pressing a manually operable actuator 141′ in a first portion subcutaneously implanted in the patient's abdominal wall AW in the form of a switch. The actuator is connected to a combined energy storage means and controller device 102. The stimulation devices 160, 161 are controlled and supplied with energy via the energy storage means and controller device 102. The device is connected to the electrical stimulation devices 160, 161 via individual lines.

Under the control of the device 102, different portions of the intestinal wall of the reservoir 140 are stimulated at different times in a predetermined stimulation pattern by means of the electrical stimulation devices 160, 161 and, thus, different sections of the intestinal reservoir 140 are constricted by such stimulation. The stimulation devices 160, 161 are specifically adapted to stimulate, over time, respectively adjacent portions of the intestinal wall of the reservoir 140 in a consecutive or wave-like manner in a direction towards the stoma 170 (or rectum/anus) to cause the reservoir 140 to be emptied. This structure allows for adapting the arrangement of the stimulation devices 160, 161 and their mode of operation to the individual form of the intestinal reservoir 140. This functionality is further enhanced where each of the stimulation devices carries 160, 161 a plurality of electrodes that are controlled individually or in groups.

As stated before, the entry valve 194 is preferably closed during the emptying of the reservoir. This is particularly important in case that all stimulation devices 160, 161 are activated simultaneously so as to constrict all sections of the reservoir 140 at the same time. Since the exit valve 194 is closed, intestinal contents cannot flow back from the reservoir into the patient's intestine but are urged towards the exit of the reservoir. An entry valve is not specifically needed when the electrical stimulation devices are activated in a consecutive or wave-like manner, as mentioned before.

In another embodiment shown in FIGS. 165A and 165B, the stimulation devices 160, 161 are specifically provided for being embedded in folds or invaginations 141 surgically created in the intestinal wall of the reservoir 140. By providing the invaginations 141 in the reservoir 140, the simulation devices 160, 161 are substantially surrounded by tissue of the reservoir 140 and, thus, contact area is increased.

Stimulation of the reservoir 140 can thus be improved. Furthermore, surrounding tissue in the abdominal cavity is not contacted by the electrodes of the stimulation devices and, thus, not influenced by the stimulation process. Fixation of the stimulation devices 160, 161 is also improved, thereby ensuring that the stimulation devices 160, 161 are precisely located over long time. The stimulation devices 160, 161 necessarily follow all movements of the intestinal wall of the reservoir 140.

Alternatively, or even in addition to the electrical stimulation type pump, a constriction type pump may be implanted in the patient's body for constricting the reservoir 140 mechanically or hydraulically from outside the intestinal wall of the reservoir 140. Examples of mechanical and hydraulic constriction type pumps will be described in more detail hereinafter in relation to FIGS. 166A, 166B and

FIGS. 167A, 167B. Where the stimulation type pump is combined with a constriction type pump, the two pumps preferably act on the same portion of the reservoir 140. In that case, it is advantageous if the constriction type pump constricts the respective portion of the reservoir 140 only partly, in order not to damage the intestine, whereas further constriction is achieved by simultaneous electrical stimulation of the same portion.

In addition, when constriction of the reservoir 140 caused by the constriction type pump is released, the stimulation type pump may, if accordingly adapted, be used to pump intestinal contents towards the exit of the reservoir 140 by, over time, stimulating different portions of the intestinal wall of the reservoir 140 in a wave-like manner in a direction of natural intestinal contents flow. In this way, filling of the reservoir 140 is supported, since intestinal contents do not remain in the area of the entrance of the reservoir 140 but are transported in the direction towards the exit.

FIGS. 166A, 166B show an embodiment of a mechanical type pump comprising mechanically acting members in the form of rollers 180, 181 for emptying the reservoir 140. The rollers 180, 181 are arranged on opposite sides of the reservoir 140 and have a length spanning the entire width of the reservoir 140, i.e. 10 cm or more. The rollers are each guided by two tracks 182a, 183a and 182b, 183b, respectively, and are driven by a motor integrated in the rollers (not shown) which preferably comprises a servo drive. The servo drive reduces the force required to move the rollers 180, 181, so that a relatively small motor can be used in exchange for a longer emptying process. The tracks 182a, 183a, 182b, 183b are arranged in pairs on opposite sides of the reservoir 140. As can be seen from FIG. 166B, the tracks have bent end portions 184a, 184b directed away from the reservoir 140 so that the rollers 180, 181 can assume an inactive position in which they do not constrict the reservoir 140. When emptying of the reservoir is desired, the rollers 180, 181 are driven along the tracks in the direction of the arrows, thereby approaching each other and constricting the reservoir 140. When the rollers are further guided by the tracks along the length of the reservoir in their proximate position, they mechanically squeeze intestinal contents in the direction towards and out of the exit of the reservoir 140. When the rollers 180, 181 have reached their final position and the reservoir 140 is emptied, they are returned to their initial inactive position at the end portions 184a, 184b of the tracks. Instead of rollers on each side of the reservoir 140, it can be sufficient to provide one or more rollers only on one side of the reservoir 140 and place a counteracting plate on the respective opposite side of the reservoir 140.

Again, emptying of the intestinal reservoir 140 can be activated by the patient pressing the manually operable actuator subcutaneously implanted in a first portion 141′ the patient's abdominal wall AW, the actuator being connected to the combined energy storage means and controller device 102. Energy is supplied from the controller device 102 to the motor or motors inside the rollers 180, 181.

FIGS. 167A, 167B show an embodiment of a hydraulic type pump comprising a hydraulically acting member 190 adapted to act on the intestinal wall of the reservoir 140 from the outside thereof. The hydraulically acting member 190 is connected to an artificial reservoir 193 supplying the hydraulically acting member 190 with hydraulic fluid. The artificial reservoir 193 is of a size sufficiently large to accommodate hydraulic fluid in an amount corresponding to the volume of the intestinal reservoir 140. The artificial reservoir 193 has a flexible wall to allow the hydraulic fluid to be drawn off from and to be filled back into the artificial reservoir 193. The hydraulically acting member 190 is of flexible material and may be tube-like or bag-like so as to accommodate therein the intestinal reservoir 140. As shown in FIG. 167B, the reservoir 140 is surrounded by the hydraulically acting member 190. The hydraulically acting member 190 is divided into a plurality of chambers, wherein a first chamber 191 and a last chamber 194 are connected to the artificial reservoir 193 by hydraulic conduits. The chambers are interconnected via connections 192, which may be simple holes acting as a throttle or may include one or more valves that are preferably automatically controlled.

Upon activation of the system by the patient using the subcutaneous actuator in the first portion 141′, emptying of the intestinal reservoir 140 is started by supplying hydraulic fluid from the artificial reservoir 193 to the first chamber 191. The next following chambers are supplied with the hydraulic fluid through the connections 192, thereby causing the hydraulically acting member 190 to be filled slowly from the first chamber 191 to the last chamber 194. The filling of the chambers occurs sequentially, with the next following chamber starting to fill before the foregoing chamber is filled completely. In this manner, intestinal contents are hydraulically squeezed out in the direction towards the exit of the reservoir 140. When the hydraulically acting member 190 is completely filled with hydraulic fluid, the reservoir 140 is completely constricted. The hydraulic fluid is then withdrawn from the chambers of the hydraulically acting member 190 back into the artificial reservoir 193 using negative pressure. The intestinal reservoir 140 may then start to fill up with intestinal contents again.

Again, this process is controlled by the controller device 102, which is connected to the artificial reservoir 193. Connected to or integrally formed with the artificial reservoir 193 is an electrically driven pump (not shown) for pumping the hydraulic fluid into and withdrawing the hydraulic fluid from the hydraulically acting member. The electrically driven pump is supplied with energy from the combined energy storage means and control device 102.

In another embodiment, each chamber of the hydraulically acting member 190 may have separate fluid connection to the artificial reservoir 193 in order to be able to be filled individually. The intestinal reservoir 140 may be emptied by consecutively filling two adjacent chambers of the hydraulically acting member 190, i.e. first filling the first and second chamber, then emptying the first chamber while filling the third chamber, then emptying the second chamber while filling the fourth chamber, and so forth. In this manner intestinal contents are squeezed towards and out of the exit of the intestinal reservoir 140.

Alternatively, instead of applying a negative pressure for evacuating the chambers, at least one valve, preferably two valves, may be provided (not shown) between the hydraulically acting member 190 and the artificial reservoir 193 which, when in an appropriate operational position, allows the hydraulic fluid to passively flow from the hydraulically acting member back into the artificial reservoir 193 when the intestinal reservoir 140 fills with intestinal contents and which, when in an appropriate other position, prevents the hydraulic fluid to flow from the hydraulically acting member back into the artificial reservoir when the intestinal reservoir is being emptied.

As in all embodiments, emptying of the reservoir 140 is coordinated with the opening and closing of the entry valve 194 and exit valves 61, 62, 63.

### Energy Transmission

An energy source may be provided for supplying energy directly or indirectly to at least one energy consuming part of the system, in particular for driving the pump or the motor of the pump. Preferably, the energy source includes a battery or an accumulator, such as one or more of a rechargeable battery and a capacitor, as an energy storage means. The energy storage means is advantageously adapted for being implanted inside the patient's body, as in the case of the afore-mentioned combined energy storage means and control device 102.

Energy is preferably transmitted wirelessly. Thus, where the energy source is provided for supplying energy directly or indirectly to at least one energy consuming part of the system, the energy source may comprise a wireless energy transmitter adapted to wirelessly transmit energy from outside the patient's body to the at least one energy consuming part. Alternatively, where the energy source includes a battery or an accumulator, in particular one which is implanted in the patient's body, the energy source may comprise a wireless energy transmitter adapted to wirelessly transmit energy from outside the patient's body to the energy storage means.

Where energy is not transmitted wirelessly, galvanic coupling elements may be provided at least between the accumulator and the energy consuming part, in particular the motor, for transmitting energy to the motor in contacting fashion.

Preferably, in order to reduce the number of parts and possibly increase the system's efficiency, the energy consuming part, in particular the motor, can be adapted to directly transform the wirelessly transmitted energy from the accumulator into kinetic energy. In the alternative, the energy consuming part will have to comprise a transforming device for transforming the wirelessly transmitted energy from the accumulator into electric energy.

Similarly, the system preferably comprises an implantable energy transforming device for transforming the wirelessly transmitted energy from outside the patient's body into energy to be stored in the accumulator of the implanted system and further comprises a wireless energy transmitter adapted to wirelessly transmit energy from outside the patient's body to said implantable energy transforming device.

It is further preferred to set up the system such that the energy consuming part is driven with the electric energy, as said energy transforming device transforms the wireless energy into the electric energy.

The energy transmitter can be adapted to generate an electromagnetic field, a magnetic field or an electrical field. The wireless energy may be transmitted by the energy transmission device by at least one wireless signal. More specifically, the energy transmitter may be adapted to transmit the energy by at least one wireless energy signal, which may comprise an electromagnetic wave signal, including at least one of an infrared light signal, a visible light signal, an ultra violet light signal, a laser signal, a microwave signal, an X-ray radiation signal, and a gamma radiation signal. Also, the wireless energy signal may comprise a sound or ultrasound wave signal. Furthermore, the wireless energy signal may comprise a digital or analog signal or a combination thereof.

### Primary Energy Source

A primary energy source may be provided for charging the energy storage means with energy from outside the patient's body. The primary energy source is preferably adapted to being mounted on the patient's body.

### Energy Transmission Feedback

A feedback subsystem, which can make part of a control device described subsequently, can advantageously be provided to wirelessly send feedback information related to the energy to be stored in the energy storage means from inside the human body to the outside thereof. The feedback information is then used for adjusting the amount of wireless energy transmitted by the energy transmitter. Such feedback information may relate to an energy balance which is defined as the balance between an amount of wireless energy received inside the human body and an amount of energy consumed by the at least one energy consuming part. Alternatively, the feedback information may relate to an energy balance which is defined as the balance between a rate of wireless energy received inside the human body and a rate of energy consumed by an energy consuming part.

### Control Unit (102)

It is advantageous to provide a control unit adapted to directly or indirectly control one or more elements of the system, such as for controlling opening of the exit valve and/or closing of the entry valve in combination with the pump, in particular in a manner such that when one of the two valves is closed, the respective other valve is open, and vice versa.

At least part of the control unit may be adapted to be implantable in the patient's body. For instance, as described before, a manually operable actuator in the form of a switch may be provided for activating the control unit 102, the switch preferably being arranged for subcutaneous implantation in an first portion so as to be operable from outside the patient's body. Alternatively, the control unit may comprise a first part adapted for implantation in the patient's body and a second part adapted to cooperate with the first part from outside the patient's body. In this case, the control unit can be adapted to transmit data from the external second part of the control unit to the implanted first part of the control unit in the same manner as energy is transmitted by said wireless energy transmitter from outside the patient's body to said implantable energy transforming device.

That is, the second part of the control unit may be adapted to wirelessly transmit a control signal to the implantable first part of the control unit for controlling the at least one energy consuming part from outside the patient's body. Also, the implantable first part of the control unit may be programmable via the second part of the control unit. Furthermore, the implantable first part of the control unit may be adapted to transmit a feedback signal to the second part of the control unit.

### Sensor

Furthermore, a physical parameter sensor adapted to directly or indirectly sense a physical parameter of the patient can be provided. The physical parameter sensor may be adapted to sense at least one of the following physical parameters of the patient: a pressure within the artificial intestine section, a pressure within the patient's natural intestine, an expansion of the artificial intestine section, a distension of an intestinal wall of the patient's natural intestine, a movement of the patient's intestinal wall.

Similarly, a functional parameter sensor adapted to directly or indirectly sense a functional parameter of the system can be provided, wherein the functional parameter sensor may be adapted to sense at least one of the following functional parameters of the system: a pressure against a part of the system such as the artificial intestine section, a distension of a part of the system such as a wall of the artificial intestine section, an electrical parameter such as voltage, current or energy balance, a position or movement of a movable part of the system.

Preferably, an indicator is coupled to the sensor or sensors, the indicator being adapted to provide a signal when a sensor senses a value for the parameter beyond a predetermined threshold value. The sensor signal may comprise at least one of the following types of signals: a sound signal, a visual signal.

### DA: Implantable Infusion Device (Basic Aspects)

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can be used for controlling, communicating with and/or operating implantable infusion devices, examples of which will now be described.

FIG. 168 shows an implantable medical device 100 in the form of a strictly mechanical, more exactly a hydro-mechanical, infusion device implanted subcutaneously under a patient's skin SK. The infusion device comprises a needle 1 having a tip end 2. Tip end 2 is closed at its distal end and has a lateral drug delivery exit port 3. Needle 1 is arranged for longitudinal displacement within an open-ended tube 4 upon activation by a drive unit D.

Tube 4 penetrates the skin SK and is attached to an extracorporeal pump P. Pump P is schematically shown and can be designed in many ways. In the embodiment shown in FIG. 168, reservoir R with infusion liquid to be delivered to the patient is part of pump P. Alternatively, reservoir R could be separate from pump P and connected thereto, e.g. as principally shown in FIG. 169. In the embodiment of FIG. 168, however, a piston 10 of pump P is manually displaceable by means of an actuator 11 in the form of a piston rod so as to pump the infusion liquid from reservoir R through tube 4 towards needle 1. Instead of being manually driven, the pump could be motor-driven, and the motor could be automatically controlled so as to deliver a certain amount of drugs at certain time intervals. Reservoir R, pump P and/or other components of the drug delivery system, such as the aforementioned motor, automatic control for the motor, etc., may alternatively be implanted along with infusion needle 1 and drive unit D. Other modifications are possible and will become apparent upon further consideration of the embodiments described later in reference to FIGS. 169 to 175.

In the infusion device shown in FIG. 168, as the pressure is increased in reservoir R by actuation of piston 10, this will result in a displacement of needle 1 against the force of a spring 5 of drive unit D. Thus, tip end 2 of needle 1 will penetrate any fibrosis having built up in front of the infusion device. When return spring 5 is completely compressed and the pressure exerted on the infusion liquid by means of piston 10 is further increased, a ball valve 6 will be displaced against a second return spring 7 which is stronger than the first return spring 5. That way, as long as the pressure is held at a sufficiently high level, infusion liquid will be pumped from reservoir R through tube 4, hollow needle 1 and the needle's exit port 3 into the patient's body. Upon pressure release, ball valve 6 will close due to return springs 5 and 7, and then needle 1 will be retracted to its initial position shown in FIG. 168.

The fit between the outer surface of needle 1 and the inner surface of tube 4 should be sufficiently tight to prevent any fibrosis from growing in.

It should be noted that the force acting on needle 1 to advance the same may be calculated as the product of the actual pressure and the cross section of needle 1. Since the cross section of a typical infusion needle is relatively small, high pressure will have to be exerted in order to penetrate any fibrosis and to overcome the counteracting forces of return springs 5 and 7. It is therefore advantageous to construct drive unit D such that two strictly separated chambers are formed in front of and behind the drive unit. Thus, when the chamber behind drive unit D is kept at low pressure, such as ambient pressure, the force acting on needle 1 would correspond to the product of the actual pressure and the entire cross section of drive unit D and, thus, be substantially higher.

This is shown in FIG. 169. Drive unit D comprises a piston 8 to which needle 1 is attached as shown in FIG. 168. Piston 8 separates a first chamber 9a in front of piston 8 and a second chamber 9b behind piston 8. While the pressure in first chamber 9a corresponds to the pressure exerted by pump P, the pressure in second chamber 9b can be kept at a lower value.

For instance, chamber 9b could be filled with a compressible gas. In that case, return spring 5 could be dispensed with as the compressed air would already create a needle retraction force.

It is, however, difficult to securely seal a gas chamber. Therefore, second chamber 9b is instead filled with liquid, such as the infusion liquid, and the liquid may be urged into a flexible volume 12. The flexible volume 12 could be of simple balloon type so as to fill up without exerting any strong counter force. Alternatively, the flexible volume 12 may comprise a gas chamber separated from the liquid of second chamber 9b by a flexible membrane. Again, return spring 5 could be dispensed with in this case.

Instead of the flexible volume 12, a conduit 13 may connect second chamber 9b with reservoir R. Thus, when needle 1 is advanced, liquid will be dispelled from second chamber 9b through conduit 13 into reservoir R, and as needle 1 is retracted by means of return spring 5, liquid will be drawn from reservoir R through conduit 13 back into second chamber 9b.

Clearly, pump P and reservoir R may be implanted into the patient's body along with drive unit D and needle 1, either remote thereof or as a single unit, if desired.

FIG. 170 shows a completely implantable, strictly mechanical infusion device to be implanted subcutaneously. The individual components of the device are contained within a unitary body 15 comprising an outer wall 16a, 16b. The volume defined by outer wall 16a, 16b is completely filled with infusion liquid. A wall portion 16a is flexible so as to allow for volume changes occurring with each injection and refill. Wall portion 16a is made from a polymer material which is self-sealing with respect to the penetration of a replenishing needle. The infusion device can thus be refilled with infusion liquid through the polymer wall portion 16a while being implanted subcutaneously.

The other wall portion 16b is rigid to provide some stability for the individual components contained within body 15. A window area 17 is formed in rigid wall portion 16b and a penetration membrane 18 is sealingly press fitted in window area 17. Penetration membrane 18 is made from a self-sealing material in respect of penetrations resulting from infusion needle 1, which infusion needle is arranged for penetrating window area 17.

Needle 1 is connected to a piston 8 separating a first chamber 9a in front of piston 8 and a second chamber 9b behind piston 8, as discussed above in reference to FIG. 169. A return spring 5 and a ball valve 6 with a return spring 7 are also provided. Openings 19 are provided to connect second chamber 9b to reservoir R so that when the pressure is raised in first chamber 9a piston 8 may dispel infusion liquid from second chamber 9b through openings 19 into reservoir R, which reservoir R is approximately at ambient pressure.

The pressure in first chamber 9a is increased by means of a pump P comprising a piston 10 formed as a unitary piece with an actuator 11 in the form of a manually actuated push button. A return spring 20 serves to urge piston 10 into its initial position shown in FIG. 170. A flow passage 21 is formed in the piston 10 with a flow constriction 22 and an exit opening 23 arranged a short distance above a housing 24 in which piston 10 is slidably arranged.

The infusion device shown in FIG. 170 functions as follows. When arranged subcutaneously with the push button 11 facing the skin, the patient may press down the push button 11 against the spring force of return spring 20. Due to flow constriction 22 in flow passage 21, the infusion liquid contained in first chamber 9a will not flow back into reservoir R through flow passage 21 but will urge piston 8 with needle 1 towards window area 17 while expelling infusion liquid from second chamber 9b through openings 19 into reservoir R. When piston 8 is in its end position and push button 11 is pressed down further, the pressure in first chamber 9a will eventually rise to a level sufficiently high to overcome the spring force of return spring 7, thereby opening ball valve 6 and allowing infusion liquid to be discharged through hollow needle 1, the tip end 2 of which has meanwhile penetrated penetration membrane 18 and any fibrosis built up thereon. Upon pressure release, ball valve 6 will immediately close and return spring 20 will urge push button 11 back into its initial position while simultaneously drawing back piston 8 with infusion needle 1 into its retracted position. Return spring 5 could be dispensed with and merely serves as a safety means. The flow passage 21 is needed to allow push button 11 to move further up even after piston 8 has reached its starting position, thereby drawing additional infusion liquid from reservoir R into first chamber 9a, which additional infusion liquid compensates the amount of infusion liquid delivered to the patient during the injection cycle.

The infusion device shown in FIG. 170 provides several advantages such as being strictly mechanical, not involving any gas chambers and not requiring any particular sealing of the piston elements 8 and 10.

In addition to the advancing and retracting capabilities of drive unit D, the drive unit of the infusion device shown in FIG. 170 further comprises means for laterally displacing the tip end of the infusion needle 1. In the particular embodiment of FIG. 170, a specific example of such lateral displacing means is shown. More particularly, needle 1 is mounted on a turntable 25 which is rotatably mounted in a circular groove 26 of second wall portion 16b. Furthermore, a guide pin 27 is securely mounted on the needle 1 to cooperate with a guide structure 28 securely fixed to rigid wall portion 16b of outer wall 15. Upon advancement or retraction of infusion needle 1, guide pin 27 will be guided in guide structure 28 and thereby laterally displace infusion needle 1, which lateral displacement causes rotation of turntable within circular groove 26.

The principle of guide structure 28 will now be described in more detail in relation to FIG. 171. Resilient flaps 28a, 28b within guide structure 28 serve to guide pin 27 through the entire guide structure 28 upon repeated advancement and retraction of infusion needle 1. Guide structure 28 is designed to provide ten different injection sites through penetration membrane 18 into a blood vessel 200 located adjacent penetration membrane 18. Where it is desired, the trajectory of guide structure 28 may include a return path 28c for guide pin 27 to return to its starting position shown in FIG. 171. Such return action will be caused by a return spring 29 fixed to the rigid second wall portion 16b.

It should be noted that all components of the infusion device shown in FIG. 170 may be made from polymer material although it is preferable that at least infusion needle 1 and return springs 5, 7, 20, 29 be made from an inert metal.

FIG. 172 shows a preferred embodiment of penetration membrane 17 in the form of a composite material. The same material can also be used for the flexible first wall portion 16a of outer body 15 or for an infusion port that will be described below in connection with another embodiment of the invention.

The composite material of penetration membrane 17 shown in FIG. 172 comprises an outer shape-giving layer 17a defining a volume in which a self-sealing soft material 17b is contained. Self-sealing soft material 17b can be of gel type having a viscosity such that it does not flow through any penetrations caused by infusion needle 1 during penetration of outer shape-giving layer 17a. Instead of a single outer shape-giving layer 17a, shape-giving layer 17a may comprise a plurality of layers. Outer shape-giving layer 17a preferably comprises silicon and/or polyurethane, since such materials can be produced to have self-sealing properties in respect of penetrations resulting from infusion needle 1.

FIG. 173 shows a fully automatic embodiment of the infusion device according to the present invention. It is to be understood, however, that manually operated elements as discussed above and automatically operating elements as discussed herein below may be combined and interchanged where this is possible. The overall drug delivery system in FIG. 173 is shown schematically, wherein all components disposed underneath the patient's skin SK are part of the implantable infusion device whereas the components above the patient's skin SK are needed to complete the drug delivery system.

A pump P driven by a motor M connects a reservoir R with an infusion needle 1 mounted on a drive unit D within a body 15 so as to penetrate upon advancement a penetration membrane 17 of body 15. A fluid conduit 4 is long enough to compensate for the advancement of infusion needle 1. While drive unit D may be activated hydraulically by forces generated by pump P, similar to the embodiments discussed above, a separate motor may be provided to actuate drive unit D. Alternatively, motor M may be designed to actuate drive unit D and any movement of drive unit D may cause pump P to pump.

Although the embodiment shown in FIG. 173 may comprise one of a great variety of reservoir types, a particular reservoir type will now be described. The volume of reservoir R shown in FIG. 173 is divided into two sections by means of a membrane 60. One section is filled with gas whereas the other section is filled with infusion liquid. An infusion port 61 allows for refilling reservoir R with infusion liquid by means of a replenishing needle. When reservoir R is in its full state, the gas section is at ambient pressure or over-pressurized. As infusion liquid is drawn from reservoir R upon each infusion cycle, the pressure in the gas section will decrease below ambient pressure, i.e. to a negative relative value. Depending upon the particular type of pump P, it may be advantageous to provide a single acting ball valve 62 to prevent any back flow from pump P to reservoir R.

Motor M is wirelessly controlled by a control unit 102 from outside the patient's body. Control unit 102 determines the time period between infusion cycles as well as the amount of infusion liquid to be injected into the patient's body upon each infusion cycle. Instead of wireless communication between control unit 102 and motor M, galvanic contacts may be provided through the skin SK. Also, the control unit 102 may be implanted along with motor M. In that case, control unit 102 is preferably programmable from outside the patient's body, either wirelessly or through galvanic contacts, so as to allow proper configuration of the control unit according to changing demands.

In addition to or instead of control unit 102, a pressure sensitive switch for activating motor M may be arranged subcutaneously.

There are various ways of providing motor M with energy. For instance, energy may be supplied from outside the patient's body either for direct use by motor M and/or for charging an accumulator A, such as a rechargeable battery and/or a capacitor. In the embodiment shown in FIG. 173, an extracorporeal primary energy source E transmits energy of a first form through the patient's skin SK to an energy transforming device T which transforms the energy of the first form into energy of a second form, such as electric energy. The electric energy is used to recharge accumulator A which provides secondary energy to motor M upon demand.

In general, external energy source E may be adapted to create an external field, such as an electromagnetic field, magnetic field or electrical field, or create a wave signal, such as an electromagnetic wave or sound wave signal. For instance, energy transforming device T as shown in FIG. 173 may act as a solar cell, but adapted to the particular type of wave signal of primary energy source E. Energy transforming device T may also be adapted to transform temperature changes into electrical energy.

Instead of an external primary energy source E, an implantable primary energy source E may be used, such as a regular long-life battery instead of accumulator A.

The energy signal can also be used to transmit control signal of the control unit 102 by appropriate modulation of the energy signal, regardless of whether the energy is transmitted wirelessly or by wire, the energy signal thereby serving as a carrier wave signal for the digital or analog control signal. More particularly, the control signal may be a frequency, phase and/or amplitude modulated signal.

FIG. 174 shows a cross sectional view of a motor-pump unit that could be used in connection with the arrangement shown in FIG. 173. This motor-pump unit is extensively described in WO 2004/0122006 A1 and the other pump units disclosed therein may be employed in connection with the present invention as well. The motor-pump unit 102 comprises a valve pump assembly, wherein a membrane pump P and a valve pump device 30 constitute two main elements of the assembly mounted in a cylindrical housing 31. Valve device 30 includes a first valve member in the form of a ceramic disc 32 stationary mounted on and fixed to housing 31, and a second valve member in the form of a ceramic disc 33 facing and touching ceramic disc 32 and rotatable relative to stationary disc 32. A motor 34 is mounted on housing 31 enclosing ceramic discs 32 and 33. Motor 34 includes a splined motor shaft coupled to corresponding splines in an under central hole in rotatable disc 33 to allow disc 33 to move somewhat in an axial direction relative to motor shaft 35, although disc 33 follows the rotation of motor 31. On motor shaft 35 is mounted a stop member 36 and a spring washer 37 that exerts a slight amount of pressure against disc 33 to urge it against stationary disc 32.

Pump P includes a pump membrane 47 that can be any kind of membrane. Preferably, membrane 47 is a metal membrane, for example a titanium membrane, or a type of coated plastic material for achieving long lifetime and avoiding diffusion of liquid through membrane 47 over time. An operation device, which in this embodiment is incorporated in the valve pump assembly, includes a cam sleeve 48 which has a cut-out groove with two opposite cam surfaces 49, a cam wheel 50, which rotates in the cut-out groove pushing against cam surfaces 49, and a pump shaft 51 connected to rotary disc 33. Cam wheel 50 is mounted via a cam wheel shaft 52 onto pump shaft 51. Pump shaft 51 rotates because it is connected to rotating disc 33 via a splined shaft 57 that is coupled to corresponding splines in an upper central hole 53 in rotatable disc 33. The described spline coupling allows disc 33 to move somewhat in an axial direction relative to pump shaft 51. Pump shaft 51 is mounted in an encapsulated ball-bearing 54 and is stationary in an axial direction with respect to ball-bearing 54. Several elongated grooves 55 on pump shaft 51 extend past ball-bearing 54 and serve as liquid flow passages between first channel 38 of stationary disc 32 and a pump chamber 56 under membrane 47.

When motor 34 is rotating, membrane 47 moves up and down. As membrane 47 moves up and down, rotatable disc 33 connects first channel 38 alternately to second and third channels 40 and 41 so that liquid is either transmitted from second channel 40 or third channel 41 to pump chamber 56 or received from pump chamber 52 by second channel 40 or third channel 41, respectively. In FIG. 174, first channel 38 is shown as being connected to second channel via opened channel 46 so that second channel receives liquid through first channel 38 from chamber 56.

The particular material selected for discs 32 and 33 is important because the selected material must be able to function using very fine tolerances without such discs sticking to one another over time. There are several materials available on the market that are suitable for this purpose, e.g. ceramic or ceramic mixed with other materials, such as carbon fiber.

FIG. 175 shows a third embodiment of the present invention with the infusion device being automatically driven and all components of the infusion device being contained within an outer body 15. The device is substantially disc-shaped with a lateral extension with a holder 90 for blood vessel 200 to maintain blood vessel 200 proximate the body 15. The infusion device shown in FIG. 175 is seen through a window in the patient's skin SK while being implanted subcutaneously. Skin SK covers the flat surface of the disc-shaped device.

Infusion needle 1 is mounted on a turntable which is part of drive unit D. Upon rotation of the turntable, the tip end of infusion needle 1 will move laterally along window 18 which is positioned proximate blood vessel 200. More particularly, infusion needle 1 is mounted on the turntable so as to be axially movable thereon back and forth such that its tip end will pass through window 18 at an inclined angle. Due to the inclination angle, the tip end of infusion needle 1 will not penetrate the opposite boundary of blood vessel 200 when it is advanced through window 18 into blood vessel 200. Holder 90 supports blood vessel 200 during penetration and injection by the needle.

Again, the interior of body 15 may serve as reservoir R. Alternatively, reservoir R may be provided as a confined chamber either within body 15 or, preferably, with a section of its periphery constituting a part of the outer wall of the body 15. If such peripheral section is made from a flexible material, such flexibility could compensate for any volume changes in reservoir R. However, at least an injection port 61 should be provided to allow for refilling reservoir R.

A pump P connects reservoir R with an infusion needle 1 which is mounted on a drive unit D for both longitudinal and lateral displacement.

Pump P is driven by a first motor M and drive unit D is driven by a separate, second motor M. Alternatively, a single motor M may be used to drive both pump P and drive unit D. Also, as described above, actuation of pump P may simultaneously cause actuation of drive unit D. Alternatively, actuation of drive unit D may cause actuation of pump P.

A long-life battery B is provided to supply the two motors M with energy. Alternatively, an accumulator, such as a rechargeable battery, may be used instead of long-life battery B.

Furthermore, a control unit 102 is provided to control the two motors M. In the embodiment shown in FIG. 175, control unit 102 is programmable from outside the patient's body by means of an external data processing device 200. Data exchange between the data processing device 200 and control unit 102 is preferably wireless through an implanted data transmission interface for wirelessly transmitting data from outside the patient's body to the control unit 102, but can be by wire through the patient's skin, if desired. Furthermore, data exchange is preferably bidirectional so as to also allow data transmission from control unit 102 to data processing device 200. Such data may include data on the performance of the device, number of infusion cycles performed, filling status of reservoir R and the like.

The infusion device in FIG. 175 further includes a feedback sensor F which—in the embodiment shown in FIG. 175—is placed in a blood vessel 200 to detect physiological parameters. Such parameters are fed to control unit 102 and might be used by a corresponding control program to prompt an infusion cycle. Alternatively or in addition, the physiological parameters may be transferred to external data processing device 200 and help the doctor in making a proper diagnosis. Eventually, the doctor will then use the data processing device 200 to adapt control unit 102 in accordance with the physiological parameters provided by feedback sensor F. Any kind of physical parameters of the patient or process parameters of the infusion device may be sent back to the control unit and the control unit may control the infusion device according to the results.

While FIG. 175 shows the third embodiment of the present invention with the infusion device being automatically driven and all components of the infusion device being contained within outer body 15, it should be understood that one or more of the various components may be implanted separate from outer body 15, such as battery B, or even outside the patient's body, such as control device C. Also, pump P and/or reservoir R may be connected separately to outer body 15, preferably attached thereto in close proximity. Components not specifically shown in FIG. 175 might be those as described in respect of the foregoing embodiments or might be different.

### DB: Implantable Infusion Device (Specific Aspects/Stimulation of Penis Erection)

A system including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant as well as for fixation and housing of operation devices for operating elements of powered/operable implantable medical devices have been described. The control unit/controller, control systems, communication systems, housing/fixation units and methods can also be used for controlling, communicating with and/or operating implantable infusion devices for the infusion of drugs which will now be described.

FIG. 176 shows the muscles of the perineum of a male. Reference numerals 1, 2 and 3 designate the ischiocavernosus muscles, bulbospongiosus muscles and superficial transverse perineal muscles, respectively. The bulbospongiosus muscle surrounds lateral aspects of the bulb of the penis at the most proximal part of the body of the penis inserting into the perineal membrane, and further surrounds the dorsal aspect of the corpus spongiosum 4 surrounding the urethra 5 and the left and right corpora cavernosa 6, 7. The ischiocavernosus 1 embraces the crus of the penis, inserting onto the inferior and medial aspects of the crus and to the perineal membrane medial to the crus. While the bulbospongiosus muscle assists the erection by compressing outflow via the deep perineal vein and by pushing blood from the bulb into the body of the penis, the ischiocavernosus muscle 1 maintains erection of the penis by compressing outflow veins and pushing blood from the root of the penis into the body of the penis. FIG. 177 is a cross-sectional view through the penis. As can be seen, the penis is composed of three cylindrical bodies of erectile cavernous tissue: the paired corpora cavernosa 6, 7 dorsally and the single corpus spongiosum ventrally. Deep arteries 9, 10 run distally near the center of the corpora cavernosa, supplying the erectile tissue in these structures. The deep arteries of the penis are the main vessels of the cavernous spaces in the erectile tissue of the corpora cavernosa and are therefore involved in the erection of the penis. They give off numerous branches that open directly into the cavernous spaces. When the penis is flaccid, these arteries are coiled, restricting blood flow.

For reasons of simplification, the following figures only display the corpora cavernosa 6, 7. FIG. 178 shows a top view on a part of the system with an implantable medical device 100 according to a first embodiment. More specifically, a single infusion needle 11 is arranged in a housing 12 with a tip end 13 of the needle 11 being positioned such that it can be advanced and retracted through a self-sealing window area 14 in the housing's 12 outer wall 15 in a longitudinal direction 16, so as to pierce the corpus cavernosum 6 or 7 located adjacent the window area 14.

Two window areas 14 are provided in the outer wall 15 of the housing 12, one adjacent each of the two corpora cavernosa 6, 7. The infusion needle is displaceable in a lateral direction 17 between the two window areas 14 by means of a drive unit D. The same drive unit D or a different drive unit may cause the infusion needle 11 to be advanced and retracted. For this purpose, the infusion needle 11 is mounted on a slide 18 for longitudinal advancement and retraction. A conduit 19 is connected to one end of the infusion needle 11 to supply infusion liquid through the infusion needle 11 to the tip end 13 thereof.

In operation, the infusion needle 11 will first be advanced with the tip end 13 thereof to penetrate one of the two self-sealing penetration windows 14, injection fluid containing a drug for stimulation of penis erection will be injected into the corpus cavernosum 7 through the infusion needle 11 and, thereafter, the infusion needle 11 will be retracted again. Upon retraction of the infusion needle, the infusion needle will be laterally displaced along the direction 17 so that the tip end 13 thereof comes to lie in front of the other of the two self-sealing window areas 14, the infusion needle 11 will be advanced again so that infusion liquid can be injected through the tip end 13 thereof into the other corpus cavernosum 7 and then the infusion needle 11 will be retracted again. At the end of this procedure, the infusion needle 11 will return to its initial position shown in FIG. 178.

The structure of the system shown in FIG. 178 may be purely mechanical. For instance, as will be described in more detail below, the pressure with which the infusion liquid is advanced through the conduit 19 towards the needle 11 may in cooperation with spring elements cause the needle 11 to be advanced, retracted and laterally displaced to the other window area 14. Thus, after two pulses of injection fluid advanced through the conduit 19 towards the needle 11, the needle 11 will automatically return to its starting position shown in FIG. 178.

However, it is likewise possible to incorporate a motor M or a plurality of motors M within the housing 15 in order to achieve the desired needle displacement by means of the drive unit D. This is schematically shown in FIG. 179 Of course, the motor M will have to be provided with energy and will need to be controlled in an appropriate manner so as to obtain the desired effect. This is not specifically shown in FIG. 179 The energy is preferably transmitted to the motor M from an energy source either remotely implanted inside the patient's body or provided externally of the patient's body.

The drive D may be configured such that after each penetration cycle (consisting of two injections) the infusion needle 11 stops at a position different from the starting position so that the tip end 13 thereof penetrates the window areas 14 in the next following injection cycle at different sites as compared to the foregoing injection cycle.

FIG. 180 shows a top view on a third embodiment which differs from the first and second embodiments in that it comprises two infusion needles 11 contained in the housing 15. Thus, when infusion liquid is guided through the conduit 19 towards the two infusion needles 11, both needles are advanced and retracted simultaneously along the direction 16, so that injection of infusion liquid occurs at exactly the same time. The drive unit D or a separate drive unit may be used to turn the turntable 20 on which the infusion needles 11 are mounted, stepwise in the direction 17 so that the window areas 14 will be penetrated by the tip end of the infusion needle 11 at different penetration sites during the next following injection cycle. Again, one or more motors M, not shown in FIG. 180, may be used for driving one or more of the components of the drive unit D.

The principle of a guide structure for laterally displacing the infusion needle will now be described in context with FIG. 181. Such guide structure may be used e.g. for each of the two infusion needles 11 shown in FIG. 180 or may also be used slightly modified for the lateral displacement of the infusion needle 11 shown in FIGS. 178 and 179 The guide structure 28 is securely fixed adjacent the self-sealing window area 14 which itself is implanted adjacent the patient's corpus cavernosum 7. The guide structure 28 comprises a guide pin 27 securely connected to the infusion needle 11 (not shown) such that the infusion needle 11 cooperates with the guide structure 28. Upon advancement or retraction of the infusion needle 11, the guide pin 27 will be guided in the guide structure 28 and thereby laterally displace the infusion needle 11, which lateral displacement causes rotation of the turntable 20 (not shown in FIG. 181). Resilient flaps 28a, 28b within the guide structure 28 serve to guide the guide pin 27 through the entire guide structure 28 upon repeated advancement and retraction of the infusion needle 11. The guide structure 28 is designed to provide different penetration sites through the self-sealing window area 14 into the corpus cavernosum 7.

Where it is desired, the trajectory of guide structure 28 may include a return path 28c for the guide pin 27 to return to its starting position shown in FIG. 181. Such return action will be caused by a return spring 29 which is permanently fixed to a rigid part of the housing 15.

The same structure can likewise be used in the embodiments shown in FIGS. 178 and 179 to displace the single infusion needle 11 laterally between the two window areas 14. Of course, the structure would have to be slightly adapted to accommodate for the larger distance to be overcome between the two window areas 14.

FIG. 182 shows a preferred embodiment of a penetration membrane to be used as the self-sealing window area 14 in the outer wall 15 of the housing 12. The penetration membrane 30 is made from a composite material. The same material can also be used for other flexible wall portions or for an infusion port that will be described below in connection with another embodiment. The composite material of penetration membrane 30 shown in FIG. 182 comprises an outer shape-giving layer 30a defining a volume in which a self-sealing soft material 30b is contained. Self-sealing soft material 30b can be of gel type having a viscosity such that it does not flow through any penetrations caused by the infusion needle 11 during penetration of the outer shape-giving layer 30a. Instead of a single outer shape-giving layer 30a, the shape-giving layer 30a may comprise a plurality of layers. The outer shape-giving layer 30a preferably comprises silicone and/or polyurethane, since such materials can be produced to have self-sealing properties in respect of penetrations resulting from the infusion needle 11.

Instead of a self-sealing membrane, the window area 14 in the outer wall 15 of the housing 12 may be formed by one or more flaps, as shown in FIG. 183. Two flaps 30′ being made from a resilient, biocompatible material are arranged so as to form a slit which is normally closed and through which the infusion needle 11 can pass when it is advanced. Upon advancement of the infusion needle 11, the needle will push aside the normally closed flaps 30′, and when the needle 11 is retracted again, the flaps 30′ will return to their normally closed position so as to form a seal against ingression of body liquid.

FIG. 184 shows a different embodiment. In this case, the self-sealing window 14 in the outer wall comprises a door 30″ which can be opened by mechanical action. In the embodiment shown, the door is formed by a flap made from a resilient, biocompatible material which keeps the window area 14 closed in its normal position. A pull wire 300 is attached to one end of the door 30″ in order to allow for opening the door by pulling the pull wire 300. The pull wire 300 or any other drive connected to the door 30″ forms part of the drive unit coupled to the infusion needle 11. For instance, as is shown in FIG. 185, the pull wire 300 may be attached directly to the infusion needle 11 so that advancement of the infusion needle 11 will simultaneously cause the door 30″ to be lifted up so that the infusion needle 11 can pass underneath the door 30″ and thus penetrate the outer wall 15 easily. Due to the resiliency of the door material, the door 30″ will automatically close when the force, such as the pulling force exerted via the pull wire 300, is released. Instead or in addition, the closing action may be supported by at least one spring element urging the door into its closed position.

FIG. 186 shows a fourth embodiment comprising a plurality of infusion needles for each of the two window areas 14. In this embodiment it is not necessary to provide a turntable by which the needles can be pivoted stepwise in order to laterally displace the needles from one penetration site to a different penetration site within the same window area 14. Instead, upon successive injection cycles a different one of the plurality of injection needles will be advanced and retracted for each of the two window areas 14. Thus, the effect achieved is the same as in the embodiment shown in FIG. 180. However, instead of the turntable 20 (FIG. 180) a valve V is needed to direct the infusion liquid to only one of the plurality of infusion needles 11 in each of the two window areas 14. More specifically, depending upon the position of the valve V, a first one of the infusion needles 11 in the first window area 14 and a first one of the plurality of the infusion needles 11 in the second window area 14 will be advanced and retracted simultaneously, and during the next following infusion cycle, another one of the plurality of infusion needles will be advanced and retracted in the two window areas 14.

FIG. 187 shows a side view of a fifth embodiment which differs from the first and second embodiments shown in FIGS. 2070678 and 179 in that the single infusion needle 11 is not only laterally displaceable in the direction 17 between the two window areas 14 but also laterally displaceable between different penetration sites 21 within the same penetration area 14. More specifically, the direction of lateral displacement of the tip end of the infusion needle 11 within each of said different penetration areas 14 is perpendicular to the direction of lateral displacement between the different penetration areas 14. To achieve this result, the drive unit D is configured to longitudinally advance and retract the infusion needle 11 along a direction 16, to pivot the infusion needle 11 by means of a turntable 20 between the two penetration areas 14 along a pivoting direction 17 and to raise or lower the infusion needle 11 along a third direction 22 perpendicular to the longitudinal direction 16. A suitable purely mechanical construction may perform this function. However, one or more motors may also be provided to perform one and/or the other of these functions.

FIG. 188 shows a side view of a sixth embodiment similar to the fifth embodiment shown in FIG. 187. In contrast to FIG. 187, the infusion needle 11 is not only laterally displaceable between different penetration sites 21 within the same penetration area 14 in a direction perpendicular to the direction of lateral displacement between the two penetration areas 14, but is also laterally displaceable within the same penetration area 14 in a direction parallel to the direction of lateral displacement between the different penetration areas 14. In other words, the tip end of the infusion needle 11 is laterally displaceable in two dimensions within the same penetration area 14.

FIG. 189 shows a seventh embodiment which enables the infusion needle 11 to be moved along a three-dimensional, spherically curved array of penetration sites. In this embodiment, a part of the housing 12, more specifically the window area 14, is spherically curved and the needle 11 is mounted in a sphere so that upon rotation of the sphere along the directions 17a and 17b the tip end 13 of the needle 11 can be moved to any position in front of the window area 14. Once an appropriate position has been adjusted for the tip end 13, the needle 11 can be advanced on the slide 18 so as to penetrate the window area 14.

Instead of accommodating the slide inside the sphere, it may likewise be mounted on the outer surface of the sphere. Similarly, the infusion needle 11 itself can be mounted on the outer surface of the sphere. The mechanism for moving the sphere along the directions 17a, 17b can be of many different types, such as mechanical by means of rollers or magnetic.

FIG. 190 shows a side view of an eighth embodiment similar to the third embodiment shown in FIG. 180. That is, two needles 11 are provided in a common housing so as to be longitudinally movable in order to advance and retract the tip ends thereof through the penetration areas 14. The infusion needles 11 is mounted on a turntable 20, as in the third embodiment of FIG. 180, so as to change the injection sites 22 within a penetration area 14 upon each injection cycle. In addition, the two injection needles can be raised and lowered along a direction 22, similar to the fifth embodiment described above in relation to FIG. 187. Again, the result is that the direction of lateral displacement of the tip ends of the two infusion needles 11 within each of the two different penetration areas 14 is perpendicular to the direction of distance between the two different penetration areas 14. Of course, this embodiment, like the sixth embodiment shown in FIG. 188, can also be modified such that the tip ends of the two infusion needles 11 are laterally displaceable in two dimensions within the same penetration area 14. This is shown in FIG. 191, displaying a side view of a ninth embodiment. In this embodiment, like in the sixth embodiment shown in FIG. 188, the tip ends of the two infusion needles 11 are laterally displaceable in two dimensions within the same penetration area 14.

FIG. 192 shows a tenth embodiment with a principle of advancing and retracting the infusion needle 11 by means of a pull wire 101. The pull wire 101 is redirected about a pin 102 such that by pulling the wire 101 at an end remotely located somewhere in the patient's body the tip end of the infusion needle 11 will be advanced through the window of the housing 12. A helical spring provides a counterforce so that the infusion needle 11 will be retracted once the pulling force on the pull wire 101 is released. This principle can be combined with other embodiments described hereinbefore and hereinafter. Instead of the helical spring 104, a second pull wire may be provided to retract the infusion needle 11. It is even possible to use a single pull wire 101 running around two pins 102 in a loop, so that pulling the wire 101 in the one direction or in the other direction will cause advancement or retraction of the infusion needle 11.

The pull wire 101 and the conduit 19 for the infusion liquid are guided in a common sheath 103. The common sheath 103 has various functions. First, it gives support to the pull wire 101 in bending sections. Second, it facilitates implantation of the conduit 19 along with the pull wire 101. Third, it protects the pull wire 101 against any build-up of fibrosis. In the case that the infusion needle 11 is long and flexibly bendable, the pull wire 101 and the long infusion needle may be guided in the common sheath 103.

FIG. 193 shows an eleventh embodiment which involves remotely actuated pull wires 105, 106 guided within a common sheath 103 along with the conduit 19 for the infusion liquid. The pull wires 105 and 106 are directly attached to the infusion needle 11 on opposite sides thereof so that the infusion needle 11 which is mounted on a turntable 20 will be laterally displaced in the one direction or in the other direction depending on whether the wire 105 or the wire 106 is pulled. Instead of using two wires 105, 106, one of the wires may be replaced with a pre-tensioning means, such as the helical spring 104 in FIG. 192. In addition, a further wire, in particular third wire (not shown), may be provided for lateral displacement of the infusion needle 11 in a further direction, so that a two-dimensional lateral displacement can be achieved by pulling the appropriate wires.

The pull wires may alternatively be attached to an element other than the infusion needle 11, provided that the infusion needle 11 is connected to such other element, so that when the other element is moved or turned by pulling one or more of the wires the tip end of the infusion needle 11 will be displaced accordingly.

In the case that a long, flexibly bendable needle is provided with the tip end thereof being arranged in a first housing for penetrating the outer wall of the first housing and the other end is arranged in a remotely implanted second housing, one can dispense with the turntable 20 and achieve accurate lateral displacement of the tip end of the needle by pulling the appropriate one of three pull wires which are attached either directly or indirectly to the circumference of the front end of the infusion needle at regularly spaced intervals.

FIG. 194 shows a twelfth embodiment with a different principle of advancing and retracting the tip end of the infusion needle, on the one hand, and laterally displacing the tip end of the infusion needle 11, on the other hand. Instead of pull wires, rotating shafts 107, 108 are provided. The drive for driving the rotating shafts 107, 108 is remotely located somewhere in the patient's body. The front ends of the rotating shafts have a threading 109, 110, e.g. in the form of a worm screw, meshing with the teeth of a rack 111, 112 formed either directly or indirectly on the infusion needle 11 and on the turntable 20, respectively. Thus, by turning the rotating shaft 107, the infusion needle 11 will advance or retract, as the case may be, due to the cooperation of the worm screw 109 and the rack 111. The gearing 109, 111 may likewise be arranged remote from the first housing, i.e. within the second housing accommodating the respective other end of the needle (which is particularly suitable when a long, flexibly bendable needle is used—not shown). Similarly, by turning the rotating shaft 108, the infusion needle 11 will be displaced laterally in the one or the other direction due to the cooperation of the worm screw 110 and the rack 112 of the turntable 20. Again, the rotating shafts 107, 108 are guided in a common sheath 103 along with the conduit 19 for the infusion liquid (or, in the case of a long, flexibly bendable infusion needle, along with the long needle—not shown).

In FIGS. 193 and 194, the action of the pull wires 105, 106 and the rotating shaft 108 make it possible to laterally displace the tip end of the infusion needle 11 between two different penetration areas and/or from a first penetration site to a second penetration site within a single penetration area.

FIG. 195 shows a first variation of an overall system comprising any one of the first to twelfth embodiment described above. Specifically shown in the variation shown in FIG. 195 is a housing 12 with a single infusion needle 11 and a drive unit D as described in relation to FIG. 187. The housing 12 is implanted with its windows areas 14 positioned adjacent the corpora cavernosa 6, 7, of which window areas 14 only one is shown in FIG. 195. A motor M is contained in the housing 12 for driving the drive unit D. While the motor M in the housing 12 may be designed to move the tip end of the infusion needle 11 in all directions as indicated in FIG. 195, it is particularly preferable in context with a long, flexibly bendable infusion needle to cause advancement and retraction of the front end of the long infusion needle by advancing and retracting the entire infusion needle from its rear end using an additional motor, so as to minimize the motor size in the housing 12 for reasons of space constraints in the injection area. The additional motor may be accommodated in a separate second housing—not shown in FIG. 195—along with the rear end of the long infusion needle and possibly along with further components remotely implanted in the patient's body. The motor M within the housing 12 (and likewise the afore-mentioned additional motor) is controlled by means of a control unit 102 constituting the implantable part of a control system which further comprises an external control unit 200 by which commands and any other kind of data can be sent to the control unit 102. For instance, the external control unit 200 may be used to initiate an injection cycle from outside the patient's body, this being done wirelessly as indicated by arrow 23. The implanted control unit 102 not only controls the motor M inside the housing 12 but also controls the energy supply from an accumulator A to the motor M inside the housing 12.

The external control unit 200 may likewise be used to program the implanted control unit 102. Also, a data transfer port for transferring data between the external control unit 200 and the implanted control unit 102 may be adapted to transfer data in both directions.

A feedback sensor F implanted inside the patient's penis is shown here as being connected to the motor M inside the housing 12 and may likewise be connected to the implantable control unit 102. The feedback sensor F can sense one or more physical parameters of the patient, such as the drug level inside the corpora cavernosa, the flow volume through the corpora cavernosa, the pressure inside the corpora cavernosa and the like. Other feedback sensors may be provided at a different location so as to sense process parameters of the system, such as electrical parameters, distention, distance and the like.

The conduit 19 connecting the needle 11 with a reservoir comprising compartments R1 and R2 and the wiring 24 for transmitting electric energy from the energy source A to the motor M inside the housing 12 are guided through a common conduit 25. Alternatively, where a long and flexibly bendable needle is used, the conduit 19 may guide the long infusion needle 11 between the reservoir and the housing 12.

In the variation of the entire system shown in FIG. 195, the reservoir comprises a first compartment R1 with e.g. a saline solution included therein, and a second compartment R2 with e.g. a drug in powder form or freeze-dried form included therein. A pump P driven by a second motor M2 is arranged to pump infusion liquid from the reservoir R1 to the infusion needle 11. The infusion liquid pumped by the pump P will pass through a mixing chamber 26 into which drugs will be released from the reservoir R2 in appropriate time coordination. The motor M2 or a different motor may cause the drugs to be released from the second reservoir R2. The motor M2 is also controlled by the control unit 102. Thus, infusion liquid pumped via the pump P from the relatively large first reservoir R1 through the mixing chamber 26, in which it is mixed with the drugs released from the second reservoir R2, will reach the infusion needle 11 which has meanwhile penetrated the self-sealing window area 14 of the housing 12 and will flow into the corpus cavernosum 7.

In addition to or instead of the control unit 102, a pressure sensitive switch for activating the motor M inside the housing 12 and/or the motor M2 may be arranged subcutaneously.

Although the embodiment shown in FIG. 195 may comprise one of a great variety of reservoir types, a particular reservoir type will now be described. The volume of the reservoir R1 is divided into two sections by means of a membrane 31. One section is filled with gas whereas the other section is filled with the infusion liquid (saline solution). An infusion port 32 allows for refilling the reservoir R1 with infusion liquid by means of a replenishing needle. When the reservoir R1 is in its full state, the gas section is at ambient pressure or over-pressurized. As infusion liquid is drawn from the reservoir R1 by means of the pump P upon each infusion cycle, the pressure in the gas section will decrease below ambient pressure, i.e. to a negative relative value. Depending upon the particular type of pump P, it may be advantageous to provide a single acting ball valve to prevent any backflow from the pump P to the reservoir R1.

There are various ways of providing the motors M and M2 with energy. In the variation shown in FIG. 195, energy is supplied from outside the patient's body either for direct use by the motors and/or for charging the accumulator A, which may be in the form of a rechargeable battery and/or a capacitor. An extracorporal primary energy source E transmits energy of a first form through the patient's skin SK to an energy transforming device T which transforms the energy of the first form into energy of a second form, such as electric energy. The electric energy is used to recharge the accumulator A which provides secondary energy to the motor M upon demand.

The external primary energy source E may be adapted to create an external field, such as an electromagnetic field, magnetic field or electrical field, or create a wave signal, such as an electromagnetic wave or sound wave signal. For instance, the energy transforming device T as shown in FIG. 195 may act as a solar cell, but adapted to the particular type of wave signal of the primary energy source E. The energy transforming device T may also be adapted to transform temperature changes into electrical energy.

Instead of the external primary energy source E, an implantable primary energy source E may be used, such as a regular long-life battery instead of the accumulator A.

The energy signal may also be used to transmit signals from the external control unit 200 by appropriate modulation of the energy signal, regardless of whether the energy is transmitted wirelessly or by wire, the energy signal thereby serving as a carrier wave signal for the digital or analog control signal. More particularly, the control signal may be a frequency, phase and/or amplitude modulated signal.

FIG. 196 shows a second variation of the entire system which basically differs from the system of FIG. 195 only in that the motor M inside the housing 12 is dispensed with. Instead, the motor M2 is used to drive the drive unit D. This is achieved by means of a rotating shaft 33 in the form of an elastically bendable worm screw, the rotating shaft 30 replacing the wiring 24 of the system shown in FIG. 195. Alternatively, where the infusion needle 11 is long and flexible, the infusion needle may be advanced by engagement of two helical gears, one of which being formed on the rear end of the infusion needle, or by a similar gearing cooperating with the infusion needle's rear end.

FIG. 197 shows a third variation of the entire system which operates purely mechanically. The reservoir R1 containing the infusion liquid, i.e. the saline solution, is of balloon type, thereby functioning both as a reservoir and as a pump if it is compressed manually from outside the patient's body. The pressure generated in the reservoir R1 will act on the reservoir R2 containing the drug. Upon a certain pressure, the drug will be released from the reservoir R2 into the mixing chamber 26 and upon further increase of the pressure the infusion liquid will be allowed to enter the mixing chamber 26, mix with the drug released from the reservoir R2, flow towards the infusion needle 11, and build up pressure on the infusion needle 11 such that the drive unit D is caused to advance the infusion needle 11 through the self-sealing window area 14 into the patient's corpus cavernosum. Once the pressure is released, the infusion needle 11 will retract automatically due to mechanical spring forces or the like and move into a different position in which it can penetrate the second of the two self-sealing window areas 14 when the reservoir R1 is compressed again. Where two infusion needles 11 are provided, a single compressing action on the reservoir R1 would be sufficient to inject the drug into both the left and right corpora cavernosa.

FIG. 198 shows a first principle of how drugs within a plurality of compartments 34 of the reservoir R2 can be released one at a time by a purely hydromechanical solution. As the infusion liquid is urged from the reservoir R1 towards the conduit 19 leading to the infusion needle or needles, it is first blocked by a spring-loaded ball valve 34 which opens only when a certain pressure is exceeded. The pressure building up in front of the ball valve 34 is guided by means of a stepper valve V sequentially onto one of a plurality of compartments 35. The compartments are each formed as a cavity 35 within a piston 36. Once a certain pressure is exceeded, the piston 36 will be pushed into a position where the compartment 35 is in flow communication with a mixing chamber 26. In the state shown in FIG. 198, three pistons 36 have already been pushed into such position. When the pressure in the reservoir R1 is further increased, the spring force of the ball valve 34 will be overcome and the infusion liquid urged from the reservoir R1 towards the conduit 19 will take with it the drug that has been released into the mixing chamber 26.

FIGS. 199 to 201 show a second principle of realizing the reservoir R2 comprising a plurality of small drug compartments 35, 35a, 35b. The drug compartments are integrally formed in a tape 201 which is wound on a first reel 202 and can be unwound from said first reel 202 onto a second reel 203. The reels 202, 203 and the tape 201 are contained in a cassette 200 which may be inserted in the entire system so as to form part of the reservoir. The cassette 200 is preferably replaceable.

As can be seen in FIG. 200, the compartments 35, 35a, 35b containing the drug e.g. in powder form or freeze-dried form are arranged in a plurality of rows as seen in the transporting direction (indicated by the arrow). However, the compartments 35 of one row are a certain distance offset in the transporting direction from the compartments 35a and 35b of the other rows. Thus, when the tape 201 is wound from reel 202 to reel 203, it is guided through a conduit 204 forming part of the cassette 200 through which the infusion liquid is pumped from the reservoir R1 to the infusion needle or needles, and the compartments 35, 35a, 35b will enter the conduit 204 one after the other.

While it is conceivable to open one of the compartments 35, 35a, 35b that has entered the conduit 204 by mechanical action, such as a hammer or piercing element, the opening of the compartments 35 in the embodiment shown in FIGS. 199 to 201 needs no further action other than winding the tape 201 onto the reel 203. That is, as can be seen from FIG. 201, when the tape 201 enters the conduit 204 through a first slit 205, the compartments 35 will not be damaged due to the fact that the slit 205 is relatively wide and is closed by two soft sealing lips 206. However, when the tape 201 exits the conduit 204 on the other side thereof, it will have to pass a narrower second slit 207 with front edges 208 that are not resilient.

The compartments 35 will therefore burst on their way out of the conduit 204 when they slip between the edges 208 of the narrow slit 207. Soft seals 209 in the slit 207 prevent liquid from leaking from the conduit 204.

The entry 210 and the exit 211 of the conduit 204 within the cassette 200 each include a valve that automatically closes when the cassette 200 is removed from the system and automatically opens when the cassette 200 is installed in the system. This allows for replacement of the cassette 200 without adversely affecting the remaining components of the overall system.

FIGS. 202 and 203 show a third principle of realizing the reservoir R2 comprising a plurality of small drug compartments 35. While FIG. 202 shows a cross-sectional plan view according to section BB in FIG. 203, FIG. 203 shows a cross-sectional side view thereof according to section AA in FIG. 202. The compartments 35 containing the drug in powder form or freeze-dried form are arranged in a rotatable plate 37. A motor M2 is provided to rotate the plate 37 about an axis 38. The motor M2 is controlled to advance the plate 37 stepwise so as to bring one compartment 35 at a time in line with the conduit 39 connecting the reservoir R1 containing the saline solution with the infusion needle or needles. Energy is supplied to the motor M2 from the accumulator A via the control unit C1.

The rotatable plate 37 is mounted in a fixed base plate 39 which itself is fixedly mounted in a housing 40 insulating the base plate 39 and the rotatable plate 37 thermally against an outer housing 42. A cooling device 41 is provided to cool a liquid surrounding the base plate 39 and rotatable plate 37 down to a temperature below 37° C. This serves to protect the drugs inside the compartment 36 from degrading too quickly. The accumulator A supplies the cooling device 41 with energy.

FIG. 204 shows a general principle of cooling the reservoir R2 containing the drug to be cooled. The cooling device 41 may be an electrothermal cooler, i.e. based on the Peltier effect consuming electric energy, or may be of the refrigerator type. Accordingly, the cold part of the cooler 41 is placed on the side to be cooled whereas the warm part of the cooling device 41 is placed on the other side so that the heat energy can be dissipated to the outside. An increased surface 41a on the warm side of the cooling device 41 serves to increase heat dissipation. Furthermore, a heat exchanging fluid may be passed through a conduit 41b along the increased surface 41a to transfer the dissipated heat energy to a remote location within the patient's body where the heat is dissipated into the patient's body through a specific heat exchanging surface 41c.

FIG. 205 shows a different principle of cooling the drugs contained in the reservoir R2. In this embodiment, two chemicals X1 and X2 are contained separate from each other in respective compartments of the cooling device 41. When the chemicals X1 and X2 are brought together, they will react with each other and such reaction will consume energy which is absorbed as thermal energy from the surroundings. By means of two pistons 41d, 41e, the chemicals X1, X2 are dispensed into a cooling line 41f in a controlled manner, which cooling line is preferably in contact with the housing 40 containing the reservoir R2. The chemical mixture X1-X2 displaced within the cooling line 41f will flow back into the chamber containing the chemicals X1, X2, but onto the other side of the pistons 41d, 41e.

A further embodiment is shown in FIG. 206. In this embodiment, again, two separate needles are provided, one infusion needle for each of the left and right corpora cavemosa. However, unlike the previously discussed embodiments, the two needles each have their own housing 12 implanted in the patient's body with their respective self-sealing window area 14 adjacent the left and right corpora cavernosa, respectively. This principle is shown in FIG. 207 in more detail with respect to one of the two needles. The drive unit D comprises a piston 50, to which the hollow infusion needle 11 is attached. The piston 50 separates a first chamber 51a in front of the piston 50 and a second chamber 51b behind the piston 50. While the pressure in the first chamber 51a corresponds to the pressure exerted by the pump P, the pressure in the second chamber 51b can be kept at a lower value. The second chamber 51b may be filled with a liquid, such as the infusion liquid, and the liquid may be urged into a flexible volume 52. The flexible volume 52 could be of simple balloon type so as to fill up without exerting any strong counterforce.

Instead of the flexible volume 52, a conduit 53 may connect the second chamber 51b with the reservoir R1. Thus, when the needle 11 is advanced, liquid will be dispelled from the second chamber 51b through the conduit 53 into the reservoir R1, and as the needle 11 is retracted by means of a return spring 55, liquid will be drawn from the reservoir R1 through the conduit 53 back into the second chamber 51b.

The injection process is carried out as follows. As the pressure is increased in the first chamber 51a by means of the pump P, the needle 11 will be displaced against the force of the spring 55 of the drive unit B. Thus, the tip end 13 of the infusion needle 11 will penetrate through the self-sealing window area 14 press-fitted into the wall 15 of the housing 12 and will further penetrate any fibrosis having built up in front of the housing. When the return spring 55 is completely compressed and the pressure built up by the pump P is further increased, a ball valve 56 will be displaced against a second return spring 57 which is stronger than the first return spring 55. That way, as long as the pressure is held at a sufficiently high level, infusion liquid will be pumped from the reservoir R1 through the conduit 19, the hollow infusion needle 11 and the needle's laterally arranged exit port into the patient's body. Upon pressure release, the ball valve 56 will close due to the return springs 55 and 57, and then the needle 11 will be retracted to its initial position shown in FIG. 198.

FIG. 208 shows the same principle, however, employing long, flexibly bendable infusion needle with only their respective front ends accommodated in the housing 12. As the pressure is increased in the first chamber 51a by means of the pump P remote from the housing 12 accommodating the tip end 13 of the infusion needle 11, the entire infusion needle 11 which is guided in the conduit 19 will be displaced against the force of the spring 55 of the drive unit B.

It may be advantageous not to pierce any living tissue by means of the injection needle 11 once it is advanced through the outer wall 15 of the housing 12. Therefore, as shown in FIG. 209, a tube 58 may be placed in front of the window area 14. The cross-sectional form of the tube 58 may be adapted to the cross-sectional form of the window area 14, i.e. where the window area 14 is rectangular, the tube 58 likewise has a rectangular cross-section.

The exit end of the tube 58 has an open area 59 sufficiently large to prevent growth of fibrosis from spanning over the open area. Fibrosis will slowly grow into the tube along the tube's inner surface, before it reaches the window area 14 after a relatively long time. The tip end 13 of the needle 11 will therefore not have to penetrate any fibrosis during the first while after implantation of the system.

Preferably, the open area 59 has an opening width of at least 3 mm. The length of the tube 58 may be in the range of 4 mm to 30 mm. The opening width 59 and the length of the tube 58 should be adjusted such that the substance injected into the tube 58 can safely seep into the patient's body. Thus, the longer the tube is, the larger the opening width thereof should be.

FIGS. 210A and 210B show a first embodiment for displacing the tip end of the infusion needle 11 in two or more different directions, i.e. a two-dimensional displacement. More specifically, FIG. 210A shows a plan view, whereas FIG. 210B shows a side elevational view schematically. As can be seen, a plate 60 to which the infusion needle 11 is fixedly mounted has a projection 61 extending into a frame 62 within which the projection 61 is free to move in any direction. Electromagnetic coils 63 are mounted on the sides of the frame 62 and are individually energizable. The electromagnetic coils 63 constitute the first part of an electromagnetic drive whereas the projection 61 is configured to constitute the second part of the electromagnetic drive. Thus, when one or more of the electromagnetic coils are energized, an electromagnetic field is created in the frame 62 and the electromagnet second part, i.e. the projection 61, will adjust its position within such field accordingly. Due to the fact that the infusion needle 11 is fixedly mounted to the plate 60, the infusion needle 11 will move along with the projection 61. This way, the infusion needle 11 can be advanced and retracted and can also be displaced laterally.

Of course, the infusion needle 11 may be attached to the electromagnetic drive in a different manner, e.g. perpendicular to the plane defined by the electromagnetic coils 63 (rather than in parallel as in FIG. 210B). As a result, the infusion needle would be laterally displaceable in a plurality of directions (rather than being advanceable and retractable).

Alternatively, the electromagnetic drive may be such as to displace the infusion needle in any lateral direction and, in addition, to advance and retract the infusion needle. This can be achieved e.g. with a structure as schematically shown in FIG. 210C relating to a second embodiment for displacing the tip end of the infusion needle 11. FIG. 210C shows an elevational side view similar to FIG. 210B, but the electromagnetic coils 63 do not define a single plane, but rather a plurality of planes is defined one above the other by providing additional electromagnetic coils 63 in a vertical direction. The top plan view would be similar to FIG. 210A. This way, the electromagnet second part 61 fixedly connected to the needle 11 moves within a three-dimensional frame 62 depending on the energization of respective ones of the magnetic coils 63.

FIGS. 211A and 211B show a plan view and a side view of a third embodiment of an electromagnetic drive for moving the infusion needle 11 in a plurality of directions. In this embodiment, the electromagnetic coils 63 constituting the electromagnet first parts are arranged in a first plane and the electromagnet second part constituted by the protrusion 61 fixedly connected to the infusion needle 11 via the plate 60 is movable in a plane in front of or behind the plane defined by the electromagnet first parts. However, the electromagnetic coils 63 are oriented differently in this third embodiment. Again, depending upon the energization of the individual electromagnetic coils, the electromagnet second part, i.e. the protrusion 61, will adjust its position in the created electromagnetic field within the frame 62.

FIG. 212 shows an overall system of the present disclosure implanted in a patient's body according to a fourth variation. This variation differs from the first to third variations described in relation to FIGS. 196 to 198 in that the infusion needle is not accommodated in a housing so as to be laterally displaceable. The infusion needle 11 is instead guided in a catheter 19 ending inside the patient's corpus cavernosum. Accordingly, two separate needles 11 are provided, which are long and flexibly bendable. They are advanced from the rear ends thereof so as to penetrate a window area 14 at the front end of the catheter, whenever a drug is injected to achieve an erection. While the variation shown in FIG. 212 is purely mechanic, similar to the variation shown in FIG. 198, the overall system may be more complex, e.g. similar to the variations described in relation to FIGS. 196 and 197.

FIG. 213 shows an overall system of the present disclosure according to a fifth variation which is similar to the afore-mentioned fourth variation, except that there is no needle provided in the catheter 19. The substances from the reservoirs R1 and R2 are delivered to the corpus cavernosum through the catheter 19. Accordingly, the catheter 19 has an open end or, more preferably, an end that can be opened when needed, e.g. mechanically as described above.

FIG. 214 shows an overall system of the present disclosure according to a sixth variation which is similar to the afore-mentioned fifth variation, except that the catheter does not end in the corpus cavernosum, but ends in close proximity thereto, such as in muscle tissue regulating blood flow through the patient's left and right corpus cavernosum and/or in another kind of tissue in close proximity to the patient's left and right corpus cavernosum. Alternatively, the catheter 19 may guide an infusion needle, such as the infusion needle 11 as described in the fourth variation in relation to FIG. 212. Thus, when the catheter 19 is implanted, the drugs are delivered through the catheter 19 or through the infusion needle 11, as the case may be, outside the corpora cavernosa.

It should be appreciated that the features of all the above described variations and embodiments may be combined and/or inter-exchanged deliberately, unless this is technically impossible.

### Long, Flexibly Bendable Infusion Needles

In particular, while most of the implantable infusion device embodiments described above relate to systems having one or more infusion needles entirely accommodated within one or more housings, it is likewise possible in all of these embodiments to employ long, flexibly bendable needles having only the front ends thereof accommodated in said housing or housings and having the respective rear ends disposed in one or more second housings remotely implanted within the patient's body. Embodiments comprising long, flexibly bendable infusion needles will be described hereinafter in relation to FIGS. 215 to DB55, which drawings otherwise correspond to FIGS. 178 to 180, 186, 189 and 191 to 196, respectively.

For reasons of simplification, the following figures only display the corpora cavernosa 6, 7. FIG. 215 shows a part of the system according to a first embodiment. More specifically, a single long and flexibly bendable infusion needle 11 is arranged with its tip end 13 in a first housing 12, wherein the tip end 13 of the needle 11 is positioned such that it can be advanced and retracted through a self-sealing window area 14 in the housing's 12 outer wall 15 in a longitudinal direction 16, so as to pierce the corpus cavernosum 6 or 7 located adjacent the window area 14.

Two window areas 14 are provided in the outer wall 15 of the first housing 12, one adjacent each of the two corpora cavernosa 6, 7. The infusion needle is displaceable in a lateral direction 17 between the two window areas 14 by means of a drive unit D. The same drive unit D or a different drive unit may cause the infusion needle 11 to be advanced and retracted. It is preferred that the drive unit for advancing and/or retracting the infusion needle, or at least the drive thereof, is disposed in a second housing which accommodates the respective other end of the infusion needle 11 and which is remotely implanted in the patient's body. For the purpose of enabling the tip end of the infusion needle to be advanced and retracted, the infusion needle 11 is mounted on a slide 18 for longitudinal movement. A conduit 19 is connected to the first housing 12 guiding the infusion needle 11 therein and, thus, protecting the infusion needle 11 against overgrowth with fibrosis. The conduit 19 continues to guide the infusion needle within the first housing up to the slide 18 and is fixed to the slide so as to perform the function of a Bowden cable system, i.e. preventing the front part of the infusion needle 11 from flexing away when the tip end 13 of the needle is advanced through the window area 14.

In operation, the infusion needle 11 will first be advanced with the tip end 13 thereof to penetrate one of the two self-sealing penetration windows 14, injection fluid containing a drug for stimulation of penis erection will be injected into the corpus cavernosum 7 through the infusion needle 11 and, thereafter, the infusion needle 11 will be retracted again. Upon retraction of the infusion needle, the infusion needle will be laterally displaced along the direction 17 so that the tip end 13 thereof comes to lie in front of the other of the two self-sealing window areas 14, the infusion needle 11 will be advanced again so that infusion liquid can be injected through the tip end 13 thereof into the other corpus cavernosum 7 and then the infusion needle 11 will be retracted again. At the end of this procedure, the infusion needle 11 will return to its initial position shown in FIG. 215.

The structure of the system shown in FIG. 215 may be purely mechanical. For instance, as will be described in more detail below, the pressure with which the infusion liquid is advanced through the needle 11 may in cooperation with spring elements cause the needle 11 to be advanced, retracted and laterally displaced to the other window area 14. Thus, after two pulses of injection fluid advanced through the conduit 19 towards the needle 11, the needle 11 will automatically return to its starting position shown in FIG. 215.

However, it is likewise possible to incorporate a motor M or a plurality of motors M within the first housing 12 in order to achieve the desired needle displacement by means of the drive unit D. This is schematically shown in FIG. 216. Of course, the motor M will have to be provided with energy and will need to be controlled in an appropriate manner so as to obtain the desired effect. This is not specifically shown in FIG. 216. The energy is preferably transmitted to the motor M from an energy source either remotely implanted inside the patient's body or provided externally of the patient's body. The number and size of the motors M in the first housing 12 should be kept at a minimum for reason of space constraints. Instead, the motors are preferably arranged remote from the first housing 12 in proximity to or within the afore-mentioned second housing.

The drive D may be configured such that after each penetration cycle (consisting of two injections) the infusion needle 11 stops at a position different from the starting position so that the tip end 13 thereof penetrates the window areas 14 in the next following injection cycle at different sites as compared to the foregoing injection cycle.

FIG. 217 shows a third embodiment comprising a long, flexibly bendable infusion needle, which differs from the first and second embodiments in that it comprises two infusion needles 11 guided within a common sheath or conduit 19 up to the first housing's outer wall 15 and guided within the first housing in separate sheaths. Thus, when both infusion needles 11 are advanced and retracted simultaneously along the direction 16, injection of infusion liquid occurs at exactly the same time. The drive unit D or a different drive unit may be used to turn the turntable 20 on which the tip ends of the infusion needles 11 are mounted, stepwise in the direction 17 so that the window areas 14 will be penetrated by the tip ends of the infusion needles 11 at different penetration sites during the next following injection cycle. Again, one or more motors M, not shown in FIG. 217, may be used for driving one or more of the components of the drive unit D.

The principle of a guide structure for laterally displacing the tip end of the infusion needle will was described above in context with FIG. DB 6. Such guide structure may be used e.g. for each of the two infusion needles 11 shown in FIG. 217 or may also be used slightly modified for the lateral displacement of the infusion needle 11 shown in FIGS. 215 and 216.

FIG. 218 shows a fourth embodiment comprising a plurality of long, flexibly bendable infusion needle for each of the two window areas 14. In this embodiment it is not necessary to provide a turntable by which the needles can be pivoted stepwise in order to laterally displace the needles from one penetration site to a different penetration site within the same window area 14. Instead, upon successive injection cycles a different one of the plurality of injection needles will be advanced and retracted for each of the two window areas 14. Thus, the effect achieved is the same as in the embodiment shown in FIG. 217.

FIG. 219 shows a fifth embodiment comprising a long, flexibly bendable infusion needle. Here the tip end of the infusion needle 11 is enabled to be moved along a three-dimensional, spherically curved array of penetration sites. In this embodiment, a part of the first housing 12, more specifically the window area 14, is spherically curved and the front part of the infusion needle 11 is mounted in a sphere so that upon rotation of the sphere along the directions 17a and 17b the tip end 13 of the needle 11 can be moved to any position in front of the window area 14. Once an appropriate position has been adjusted for the tip end 13, the needle 11 can be advanced on the slide 18 so as to penetrate the window area 14. Instead of accommodating the slide inside the sphere, it may likewise be mounted on the outer surface of the sphere. Similarly, the infusion needle 11 itself can be mounted on the outer surface of the sphere. The mechanism for moving the sphere along the directions 17a, 17b can be of many different types, such as mechanical by means of rollers or magnetic.

FIG. 220 shows a sixth embodiment comprising a long, flexibly bendable infusion needle, with a principle of advancing and retracting the infusion needle 11 by means of a pull wire 101. The pull wire 101 is redirected about a pin 102 such that by pulling the wire 101 at an end remotely located somewhere in the patient's body the tip end of the infusion needle 11 will be advanced through the window of the housing 12. A helical spring provides a counterforce so that the infusion needle 11 will be retracted once the pulling force on the pull wire 101 is released. This principle can be combined with other embodiments described hereinbefore and hereinafter. Instead of the helical spring 104, a second pull wire may be provided to retract the infusion needle 11. It is even possible to use a single pull wire 101 running around two pins 102 in a loop, so that pulling the wire 101 in the one direction or in the other direction will cause advancement or retraction of the infusion needle 11.

The pull wire 101 and the infusion needle are guided in a common sheath 103. The common sheath 103 has various functions. First, it gives support to the pull wire 101 in bending sections. Second, it facilitates implantation of the infusion needle 11 along with the pull wire 101. Third, it protects the pull wire 101 against any build-up of fibrosis.

FIG. 221 shows a seventh embodiment comprising a long, flexibly bendable infusion needle, which involves remotely actuated pull wires 105, 106 guided within a common sheath 103 along with the infusion needle 11. The pull wires 105 and 106 are directly attached to the front end of the infusion needle 11 on opposite sides thereof so that the tip end of the infusion needle 11 which is mounted on a turntable 20 will be laterally displaced in the one direction or in the other direction depending on whether the wire 105 or the wire 106 is pulled. Instead of using two wires 105, 106, one of the wires may be replaced with a pretensioning means, such as the helical spring 104 in FIG. 220. In addition, a further wire, in particular third wire (not shown), may be provided for lateral displacement of the infusion needle 11 in a further direction, so that a two-dimensional lateral displacement can be achieved by pulling the appropriate wires. In particular, due to the fact that the infusion needle 11 is long and flexibly bendable, one can dispense with the turntable 20 and achieve accurate lateral displacement of the tip end of the infusion needle 11 by pulling the appropriate one of three pull wires which are attached either directly or indirectly to the circumference of the front end of the infusion needle at regularly spaced intervals.

The pull wires may alternatively be attached to an element other than the infusion needle 11, provided that the infusion needle 11 is connected to such other element, so that when the other element is moved or turned by pulling one or more of the wires the tip end of the infusion needle 11 will be displaced accordingly.

FIG. 222 shows an eighth embodiment comprising a long, flexibly bendable infusion needle, with a different principle of advancing and retracting the tip end of the infusion needle, on the one hand, and laterally displacing the tip end of the infusion needle 11, on the other hand. Instead of pull wires, rotating shafts 107, 108 are provided. The drive for driving the rotating shafts 107, 108 is remotely located somewhere in the patient's body. The front ends of the rotating shafts have a threading 109, 110, e.g. in the form of a worm screw, meshing with the teeth of a rack 111, 112 formed either directly or indirectly on the infusion needle 11 and on the turntable 20, respectively. Thus, by turning the rotating shaft 107, the infusion needle 11 will advance or retract, as the case may be, due to the cooperation of the worm screw 109 and the rack 111. The gearing 109, 111 may likewise be arranged remote from the first housing, i.e. within the second housing accommodating the respective other end of the needle. Similarly, by turning the rotating shaft 108, the infusion needle 11 will be displaced laterally in the one or the other direction due to the cooperation of the worm screw 110 and the rack 112 of the turntable 20. Again, the rotating shafts 107, 108 are guided in a common sheath 103 along with the infusion needle 11.

In FIGS. 221 and 222, the action of the pull wires 105, 106 and the rotating shaft 108 make it possible to laterally displace the tip end of the infusion needle 11 between two different penetration areas and/or from a first penetration site to a second penetration site within a single penetration area.

FIG. 223 shows a first variation of an overall system comprising any one of the first to eighth embodiment comprising embodiment comprising one or a plurality of long, flexibly bendable infusion needles, as described above. Specifically shown in the variation shown in FIG. 223 is a first housing 12 accommodating the tip end of a single infusion needle 11 and a drive unit D as described in relation to FIG. 187. The housing 12 is implanted with its window areas 14 positioned adjacent the corpora cavernosa 6, 7, of which window areas 14 only one is shown in FIG. 223. A motor M is contained in the housing 12 for driving the drive unit D. While the motor M in the housing 12 may be designed to move the front end of the infusion needle 11 in all directions as indicated in FIG. 223, it is possible and even preferable to cause advancement and retraction of the front end of the infusion needle by advancing and retracting the entire infusion needle from its rear end using an additional motor, so as to minimize the motor size in the housing 12 for reasons of space constraints in the injection area. The additional motor may be accommodated in a separate second housing—not shown in FIG. 223—along with the rear end of the infusion needle and possibly along with further components remotely implanted in the patient's body.

The motor M within the housing 12 (and likewise the afore-mentioned additional motor) is controlled by means of a control unit 102 constituting the implantable part of a control system which further comprises an external control unit 200 by which commands and any other kind of data can be sent to the control unit 102. For instance, the external control unit 200 may be used to initiate an injection cycle from outside the patient's body, this being done wirelessly as indicated by arrow 23. The implanted control unit 102 not only controls the motor M inside the housing 12 but also controls the energy supply from an accumulator A to the motor M inside the housing 12.

The conduit 19 guides the infusion needle 11 from a reservoir comprising compartments R1 and R2 and accommodates the wiring 24 for transmitting electric energy from the energy source A to the motor M inside the housing 12.

In all other respects, the system of FIG. 223 corresponds to the one shown and discussed in relation to FIG. 195.

FIG. 225 shows a second variation of the entire system which basically differs from the system of FIG. 223 only in that the motor M inside the housing 12 is dispensed with. Instead, the motor M2 is used to drive the drive unit D. This is achieved by means of a rotating shaft 33 in the form of an elastically bendable worm screw, the rotating shaft 30 replacing the wiring 24 of the system shown in FIG. 223. Alternatively, since the infusion needle 11 is long and flexible, the infusion needle may be advanced by engagement of two helical gears, one of which is formed on the rear end of the infusion needle, or by a similar gearing cooperating with the infusion needle's rear end.

FIG. 225 shows a third variation of the entire system which operates purely mechanically. The reservoir R1 containing the infusion liquid, i.e. the saline solution, is of balloon type, thereby functioning both as a reservoir and as a pump if it is compressed manually from outside the patient's body. The pressure generated in the reservoir R1 will act on the reservoir R2 containing the drug. Upon a certain pressure, the drug will be released from the reservoir R2 into the mixing chamber 26 and upon further increase of the pressure the infusion liquid will be allowed to enter the mixing chamber 26, mix with the drug released from the reservoir R2, flow towards the long, flexibly bendable infusion needle 11, and build up pressure on the infusion needle 11 such that the drive unit D is caused to advance the infusion needle 11 through the self-sealing window area 14 into the patient's corpus cavernosum. Once the pressure is released, the infusion needle 11 will retract automatically due to mechanical spring forces or the like and move into a different position in which it can penetrate the second of the two self-sealing window areas 14 when the reservoir R1 is compressed again. Where two long, flexibly bendable infusion needles 11 are provided, a single compressing action on the reservoir R1 would be sufficient to inject the drug into both the left and right corpora cavernosa.

### E: Lubrication of a Synovial Joint

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control of an implantable medical device 100 for lubrication of a synovial joint. Examples of such devices for lubrication of joints will now be described.

In the following a detailed description of preferred embodiments will be given. In the drawing figures, like reference numerals designate identical or corresponding elements throughout the several figures. It will be appreciated that these figures are for illustration only and are not in any way restricting the scope. Thus, any references to direction, such as “up” or “down”, are only referring to the directions shown in the figures. Also, any dimensions etc. shown in the figures are for illustration purposes.

Please note that any embodiment or part of embodiment as well as any method or part of method could be combined in any way. All examples herein should be seen as part of the general description and therefore possible to combine in any way in general terms.

FIG. 226 shows a patient's body with an implanted lubrication device consisting of a main body 1401 and two fluid connection tubes 1402 that transport a lubricating fluid stored in a reservoir into the joints to be lubricated, here a hip joint and a knee joint. For this reason, the main body 1401 comprises a reservoir for storing the lubricating fluid and may also comprise further components, such as a pump, a motor, a control unit or the like. The lubrication device, i.e. all its components, is fully implantable into the patient's body such that the joint can be appropriately lubricated post-operatively independently of any extracorporeal components or injections, which significantly reduces the infection risk for the patient. Depending on the type of joint and on the severity of the damage to the joint, a joint may be lubricated intermittently/periodically, continuously, or as required, e.g. depending on a lubricating fluid level within the joint. Generally, the main body 1401 of the lubrication device may be implanted subcutaneously so that it is easily accessible, e.g. for refilling the reservoir or setting up functionalities or modes of operation via a wireless control unit or the like.

In FIGS. 227A and 227B the two lubricated joints shown in FIG. 226, the hip joint and the knee joint, are illustrated respectively in greater detail. In FIGS. 227A and 1B it can be seen that the fluid connection tube 1402 has at its end an infusion member being inserted into the joint space which finally brings the lubricating fluid into the joint. FIG. 227A shows an infusion needle 1403 which is injected through the joint capsule into the joint space of the hip joint. The infusion needle 1403 may, in connection with a drive mechanism (not shown), be advanced into the joint space and retracted from it in order to intermittently lubricate the joint. Alternatively, in FIG. 227B, the infusion member is an infusion tube 1404 that is permanently placed in the joint space such that a continuous flow of lubricating fluid reaches the joint. The material of the infusion tube 1404 may be a soft material not or only minimally disturbing the joint in its regular operation. A drive mechanism is not required for the infusion tube 1404 of FIG. 227B.

Generally, there are two basic methods for implanting the lubrication device, a conventional method in which an area of the joint is dissected free and the infusion needle 1403 or infusion tube 1404 is arranged at the free-dissected area, and a laparoscopic method in which a cavity at the joint is expanded laparoscopically and the infusion needle 1403 or infusion tube 1404 is placed in the cavity through laparoscopic trocars. If the fluid connection tube 1402 ends in an infusion needle 1403, as shown in FIG. 227A, the infusion needle 1403 is placed in close relation to the joint capsule or into a hole in the capsule in such a way that a drive mechanism of the needle may introduce and retract the infusion needle 1403 intermittently into and out of the joint space such that lubricating fluid stored in the reservoir is intermittently injected. If, alternatively, the fluid connection tube 1402 ends in an infusion tube 1404, as shown in FIG. 227B, a permanent hole is created in the joint capsule in which the tube is continuously placed such that the lubricating fluid may be continuously injected into the joint.

FIG. 227C shows a medical device according to an embodiment in which the medical device comprises a first artificial contacting surface 1101 adapted to replace the distal surface of the femoral bone 102, being part of the knee joint. The first artificial contacting surface 1101 could be adapted to replace the surface of the lateral condyle, the medial condyle or both the lateral and medial condyles. The medical device of FIG. 227C further comprises a second artificial contacting surface 1102 being adapted to replace the contacting surface of the tibia bone being the other contacting surface of the knee joint.

The implantable medical device comprises an inlet 1104; 1123 adapted to receive a lubricating fluid from a reservoir 1108, which according to this embodiment is placed on the rear side of the tibia bone 102 and the rear side of the femoral bone 102, respectively. The reservoir 1108 is according to this embodiment adapted to be refilled by means of an injection port 1107 being placed in fluid contact with the reservoir 1108. The reservoir 1108 supplies the inlet 1104; 1123 with a lubricating fluid through a conduit 1106 which supplies a fluid connection between the medical device and the reservoir 1108. The reservoir is according to this embodiment adapted to be placed under pressure through said injection port 1108 comprising chamber for pressurized gas which is further compressed when the reservoir 1108 is being filled through the injection port 1107. The inlet 1104; 1123 transports the lubricating fluid to a channel 1105 which is at least partly integrated in said artificial contacting surfaces 1101, 1102. According to the embodiment of FIG. 226 the channel 1105 is fully integrated in the medical device. The channel 1105 distributes the lubricating fluid over the artificial contacting surfaces 1101, 1102 and thereby lubricates the artificial contacting surfaces 1101, 1102 and improves the function thereof by reducing the friction.

The implantable medical device could just as well be adapted to be implanted in the knee joint of another mammal, such as a horse.

FIG. 227D shows the implantable medical device according to an embodiment where the medical device is adapted to replace parts of the hip joint. The medical device comprises a plurality of channels 1105 adapted to lubricate the artificial contacting surface 1103a of the hip joint by a lubricating fluid being injected to the channel through a conduit 1106 placed centrally in the implantable medical device.

The conduit 1106 places the plurality of channels 1105 in fluid connection with a reservoir 1108 which is located in the stem part, adapted for fixation in the femoral bone of a human patient, of said medical device. The conduit 1106 transports lubricating fluid to the inlet 1123 for further distribution to the channels 1105. The reservoir 1108 according to the embodiment of FIG. 227D is spring loaded by a spring 1109 which pushes a movable wall portion in the shape of a piston 1110 for placing said lubricating fluid under pressure. The reservoir 1108 is adapted to be refilled through the injection port 1107, which is placed on the lateral side of the medical device. The injecting of lubricating fluid through the injection port 1107 compresses the spring 1109 which thereby places the lubricating fluid under pressure, which pressure presses the lubricating fluid through the conduit 1106 and to the channels 1105 for lubricating the hip joint of a human patient. The spring loaded reservoir 1108 could be replaced by other types of reservoirs adapted to place a pressure on the lubricating fluid, such as a reservoir 1108 comprising a chamber filled with a pressurized gas which is further pressurized by the injecting of a lubricating fluid through an injection port, it is furthermore conceivable that the reservoir 1108 is an elastic reservoir in which case the elastic properties of the elastic reservoir pressurized the lubricating fluid. FIG. 227E illustrates an implanted lubrication device and its main components. The lubrication device of FIG. 227E comprises a reservoir R for storing the lubricating fluid and a fluid connection tube 1402 that connects the reservoir R with an infusion tube 1404 placed with its open end permanently within a joint space. In order to create an appropriate pressure for forcing the lubricating fluid from the reservoir R through the fluid connection tube 1402 and the injection tube 1404 into the joint, a gas chamber 1407 is arranged within the reservoir R that may produce the required pressure upon expanding its volume. Further, the reservoir is subcutaneously implanted such that a refill injection port 1406 arranged in the outer wall of the reservoir R is accessible through the patient's skin SK such that lubricating fluid can be replenished into the reservoir R by a syringe injected through the patient's skin SK. The refill injection port 1406 may thus be made of an appropriate membrane, e.g. a polymer material, which is self-sealing with respect to the penetration of an injection syringe.

FIG. 227F shows another embodiment of the lubrication device according to the present invention. A pump P driven by a motor M connects a reservoir R with a circular fluid connection tube 1402 consisting of two tube portions 1402a, 1402b that form a full circular flow path for the lubricating fluid via the reservoir R and the lubricated Joint. Each of the two tube portions 1402a, 1402b comprises an individual infusion tube 1404a, 1404b inserted into the joint space, whereas the lubricating fluid stored in the reservoir is introduced into the joint space via the tube portion 1402a with the infusion tube 1404a, while the used lubricating fluid is led from the joint back to the reservoir via the tube portion 1402b with the infusion tube 1404b via a filter device 1427 with a filter 1428 placed within the flow path partially defined by tube portion 1402b. Under the pressure created by the pump P the lubricating fluid is circulated continuously within the circular flow path formed by the fluid connection tube portions 1402a, 1402b, such that the lubricating fluid may be at least partly re-used after passing the joint. However, in order to enable re-usage of the lubricating fluid flowing out of the joint and into infusion tube 1404b, possible soiling and impurities or other foreign particles which have been added to the lubricating fluid on its way through the joint are removed by the filtering device 1427 in order to secure the quality and desired effects of the lubricating fluid upon re-usage. The filtering device 1427 has a filter 1428 which is placed within the flow path such that the complete lubricating fluid passes through the filter. The filtering device 1427 is adapted to regularly clean the filter 1428 by removing the filtered particles from the filter 1428 and depositing them in a sealed deposition space 1433. Alternatively, the removed particles can also be given back to the patient's body, e.g. into a blood vessel or the like.

Although the embodiment shown in FIG. 227F may comprise a great variety of reservoir types, a particular reservoir type will be described below. The volume of reservoir R shown in FIG. 227F is divided into two sections by means of a membrane 1429. One section is filled with gas, whereas the other section is filled with lubricating fluid. A refill injection port 1430 allows for refilling reservoir R with infusion liquid by means of a replenishing needle through the patient's skin SK. When reservoir R is in its full state, the gas section is at ambient pressure or over-pressurized. As lubricating fluid is drawn from reservoir R upon each lubrication cycle, the pressure in the gas section will decrease below ambient pressure, i.e. to a negative relative value. Depending upon the particular type of pump P, it may be advantageous to provide an active ball valve 1431 to prevent any back-flow from pump P to reservoir R and another active ball valve 1432 to prevent any back-flow from the reservoir R into the fluid connection conduit 1402b.

Motor M is wirelessly controlled by a control unit 102 implanted in the patient's body as well. However, it is also possible to place the control unit 102 outside the patient's body as an external control unit 200 and establish a wireless communication between control unit 102 and motor M or provide galvanic contacts through the patient's skin. Preferably, the control unit 102 is implanted along with motor M, in which case control unit 102 is preferably programmable from outside the patient's body, either wirelessly or through galvanic contacts, so as to allow proper configuration of the control unit according to changing demands. Control unit 102 determines the time period between the infusion cycles as well as the amount of lubricating fluid to be injected into the space upon each infusion cycle. In addition to or instead of control unit 102, a pressure sensitive switch for activating motor M may be arranged subcutaneously.

There are various ways of providing motor M with energy. For instance, energy may be supplied from outside the patient's body e.g. for charging an accumulator A, such as a rechargeable battery and/or a capacitor. In the embodiment shown in FIG. 227F, an extracorporeal primary energy source E transmits energy of a first form through the patient's skin SK to an energy transforming device T which transforms the energy of the first form into energy of a second form, such as electric energy. The electric energy is used to recharge accumulator A which provides secondary energy to motor M upon demand.

In general, external energy source E may be adapted to create an external field, such as an electromagnetic field, magnetic field or electric field, or create a wave signal, such as an electromagnetic wave or sound wave signal. For instance, energy transforming device T as shown in FIG. 227F may act as a solar cell, but be adapted to the particular type of wave signal of primary energy source E. Energy transforming device T may also be adapted to transform temperature changes into electric energy.

Instead of an external primary energy source E, an implantable primary energy source E may be used, such as a regular long-life battery instead of accumulator A. The energy signal can also be used to transmit a control signal of the control unit 102 by appropriate modulation of the energy signal, regardless of whether the energy is transmitted wirelessly or by wire, the energy signal thereby serving as a carrier wave signal for the digital or analog control signal. More particularly, the control signal may be a frequency, phase and/or amplitude modulated signal. FIG. 228A shows in further detail an implanted lubrication device comprising an infusion needle 1403 having a tip end 1408. Tip end 1408 is closed at its distal end and has a lateral lubricating fluid delivery exit port 1409. Needle 1403 is arranged for longitudinal displacement within an open-ended fluid connection tube 1402 upon activation by a drive mechanism D.

The fluid connection tube 1402 is attached to an implanted pump P. Pump P is schematically shown and can be designed in many ways. In FIG. 228A, reservoir R holding the lubricating fluid to be injected into a patent's joint space is part of pump P. Alternatively, reservoir R could be separate from pump P and connected thereto, e.g. as basically shown in FIG. 228B. In FIG. 228A, however, a movable or flexible wall 1410 of a pump P, which may be realized as a piston or the like, is electrically (or manually) displaceable so as to intermittently pump lubricating fluid from reservoir R through fluid connection tube 1402 towards infusion needle 1403. The pump P could e.g. be motor-driven, and the motor could be automatically controlled so as to intermittently inject a certain amount of lubricating fluid at certain time intervals via the infusion needle 1403 into the joint space. Reservoir R, pump P and/or other components of the implanted lubrication device, such as the aforementioned motor, an automatic control for the motor, etc., are preferably implanted along with infusion needle 1403 and drive mechanism D. Of course, other appropriate modifications are possible, as will become apparent upon further consideration of other embodiments of the present invention.

In the lubrication device shown in FIG. 2A, as the pressure is increased in reservoir R by actuation of the movable/flexible wall 1410 this will result in a displacement of infusion needle 1403 against the force of a spring 1411 of drive mechanism D. Thus, tip end 1408 of infusion needle 1403 will penetrate into the joint space to be lubricated. When return spring 1411 is completely compressed and the pressure exerted on the lubricating fluid by means of the moving/flexible wall 1410 is further increased, a ball valve 1412 will be displaced against a second return spring 1413 which is stronger than the first return spring 1411. That way, as long as the pressure is held at a sufficiently high level, lubricating fluid will be pumped from reservoir R through fluid connection tube 1402, hollow infusion needle 1403 and the needle's exit port 1409 into the patient's joint space. Upon pressure release, ball valve 1412 will close due to return springs 1411 and 1413, and then infusion needle 1403 will be retracted to its initial position as shown in FIG. 228A. This process will be periodically repeated depending on the condition and type of the joint to be lubricated such that an intermittent lubrication of the particular joint is achieved.

It should be noted that the force acting on infusion needle 1403 to advance the same may be calculated as the product of the actual pressure and the cross section of needle 1403. Since the cross section of a typical infusion needle is relatively small, high pressure will have to be exerted in order to penetrate into the joint space and to overcome the counteracting forces of return springs 1411 and 1413.

It is therefore advantageous to construct drive mechanism D such that two strictly separated chambers are formed in front of and behind the drive mechanism. Thus, when the chamber behind drive mechanism D is kept at low pressure, such as ambient pressure, the force acting on infusion needle 1403 would correspond to the product of the actual pressure and the entire cross section of drive mechanism D and, thus, be substantially higher.

This is shown in FIG. 228B. Drive mechanism D comprises a piston 1414 to which infusion needle 1403 is attached as shown in FIG. 229. Piston 1414 separates a first chamber 1415a in front of piston 1414 and a second chamber 1415b behind piston 1414. While the pressure in first chamber 1415a corresponds to the pressure exerted by pump P, the pressure in second chamber 1415b can be kept at a lower value. For instance, chamber 1415b could be filled with a compressible gas. In that case, return spring 1411 could be dispensed with as the compressed air would already create a needle retraction force.

It is difficult to securely seal a gas chamber, however. Therefore, second chamber 1415b is instead filled with fluid, such as the lubricating fluid, and the liquid may be urged into a flexible volume 1416. The flexible volume 1416 could be of simple balloon type so as to fill up without exerting any strong counterforce. Alternatively, the flexible volume 1416 may comprise a gas chamber separated from the fluid of second chamber 1415b by a flexible membrane. Again, return spring 1411 could be dispensed with in this case.

Instead of the flexible volume 1416, a conduit 1417 (acting as fluid connection tube 1402) may connect second chamber 1415b with reservoir R. Thus, when infusion needle 1403 is advanced, fluid will be expelled from second chamber 1415b through conduit 1417 into reservoir R, and as infusion needle 1403 is retracted by means of return spring 1411, fluid will be drawn from reservoir R through conduit 1417 back into second chamber 1415b. Pump P and reservoir R are be implanted into the patient's body along with drive mechanism D and needle 1403, either remote thereof or as a single unit, if desired.

FIG. 228C shows a very compact lubrication device to be implanted subcutaneously and in close vicinity and in an appropriate relative position to the joint to be lubricated, such that the needle 1403 may intermittently advance into the Joint upon activation by the drive mechanism D. The individual components of the device are contained within a unitary body 1418 comprising an outer wall 1419a, 1419b. The volume defined by outer wall 1419a, 1419b is completely filled with lubricating fluid. A wall portion 1419a is flexible so as to allow for volume changes occurring with each injection and refill. Wall portion 1419a is made from a polymer material which is self-sealing with respect to the penetration of an infusion needle 1403. The lubrication device can thus be refilled with lubricating fluid through the polymer wall portion 1419a while being implanted subcutaneously.

The other wall portion 1419b is rigid to provide some stability for the individual components contained within body 1418. A window area 1420 is formed in rigid wall portion 1419b and a penetration membrane 1421 is sealingly press fitted in window area 1420. Penetration membrane 1421 is made from a self-sealing material in respect of penetrations resulting from infusion needle 1403, which infusion needle is arranged for penetrating window area 1420 and thereby penetrating into the joint space to be lubricated.

Needle 1403 is connected to a piston 1414 separating a first chamber 1415a in front of piston 1414 and a second chamber 1415b behind piston 1414, as discussed above in reference to FIG. 228B. A return spring 1411 and a ball valve 1412 with a return spring 1413 are also provided. Openings 1422 are provided to connect second chamber 1415b to reservoir R so that when the pressure is raised in first chamber 1415a piston 1414 may expel lubricating fluid from second chamber 1415b through openings 1422 into reservoir R, which reservoir R is approximately at ambient pressure.

The pressure in first chamber 1415a is increased by means of a pump P comprising a movable/flexible wall 1410 moved forth and back by an appropriate drive mechanism, motor or the like.

A flow passage 1423 is formed in a housing 1424 in which piston 1410 is slidably arranged. The flow passage has a flow constriction 1425 and an exit opening 1426 within the housing 1424.

The infusion device shown in FIG. 228C functions as follows. When the movable/flexible wall 1410 is actuated (i.e. moved in the direction of the arrow), the lubricating fluid contained in first chamber 1415a will not flow back into reservoir R through flow passage 1423, due to flow constriction 1425 in flow passage 1423, but will urge piston 1412 with needle 1403 towards window area 1420 while expelling lubricating fluid from second chamber 1415b through openings 1422 into reservoir R. When piston 1412 is in its end position and the movable/flexible wall 1410 is further moved in arrow direction, the pressure in first chamber 1415a will eventually rise to a level sufficiently high to overcome the spring force of return spring 1413, thereby opening ball valve 1412 and allowing lubricating fluid to be discharged through hollow needle 1403, the tip end 1408 of which has meanwhile penetrated membrane 1418 and the joint at which the body 1418 of the lubrication device is appropriately positioned. Upon a release of the pressure in the first chamber 1415a due to backward sliding of movable/flexible wall 1410, ball valve 1412 will immediately close and piston 1412 with infusion needle 1403 will be simultaneously drawn back into its retracted position. The flow passage 1423 is needed to allow movable/flexible wall 1410 to move further backwardly even after piston 1412 has reached its starting position, thereby drawing additional lubricating fluid from reservoir R into first chamber 1415a, which additional lubricating fluid compensates the amount of lubrication liquid delivered to the patient during the intermittent injection cycles. In addition to the intermittent advancing and retracting capabilities of drive mechanism D, the drive mechanism of the lubrication device shown in FIG. 228C may further comprise means for laterally displacing the tip end 1408 of infusion needle 1403 as to prevent fibrosis or the like.

The lubrication device shown in FIG. 228C provides several advantages such as not involving any gas chambers and not requiring any particular sealing of movable/flexible wall 1410 and piston element 1412. It should be noted that all components of the infusion device shown in FIG. 228B may be made from polymer material, although it is preferable that at least infusion needle 1403 and return springs 1411, 1413 be made from an inert metal.

FIG. 228D shows a cross-sectional view of a motor-pump unit that could be used in connection with the arrangement shown in FIG. 227F. This motor-pump unit is extensively described in WO 2004/012806 A1 and the other pump units disclosed therein may be employed in connection with the present invention as well. The motor-pump unit comprises a valve pump assembly, wherein a membrane pump P and a valve pump device 1434 constitute two main elements of the assembly mounted in a cylindrical housing 1435. Valve device 1434 includes a first valve member in the form of a ceramic disc 1436 stationary mounted on and fixed to housing 1435, and a second valve member in the form of a ceramic disc 1437 facing and touching ceramic disc 1436 and rotatable relative to stationary disc 1436. A motor 1438 is mounted on housing 1435 enclosing ceramic discs 1436 and 1437. Motor 1438 includes a splined motor shaft coupled to corresponding splines in a lower central hole in rotatable disc 1437 to allow disc 1437 to move somewhat in an axial direction relative to motor shaft 1439, although disc 1437 follows the rotation of motor 1435. On motor shaft 1439 there are mounted a stop member 1440 and a spring washer 1441 that exerts a slight amount of pressure against disc 1437 to urge it against stationary disc 1436.

Pump P includes a pump membrane 1451 that can be any kind of membrane. Preferably, membrane 1451 is a metal membrane, for example a titanium membrane, or a type of coated plastic material for achieving long life and avoiding diffusion of liquid through membrane 1451 overtime. An operation device, which in this embodiment is incorporated in the valve pump assembly, includes a cam sleeve 1452 which has a cut-out groove with two opposite cam surfaces 1453, a cam wheel 1454, which rotates in the cut-out groove pushing against cam surfaces 1453, and a pump shaft 1455 connected to rotary disc 1437. Cam wheel 1454 is mounted via a cam wheel shaft 1456 onto pump shaft 1455. Pump shaft 1455 rotates because it is connected to rotating disc 1437 via a splined shaft 1461 that is coupled to corresponding splines in an upper central hole 1461 in rotatable disc 1437. The described spline coupling allows disc 1437 to move somewhat in an axial direction relative to pump shaft 1455. Pump shaft 1455 is mounted in an encapsulated ball-bearing 1458 and is stationary in an axial direction with respect to ball-bearing 1458. Several elongated grooves 1459 on pump shaft 1455 extend past ball-bearing 1458 and serve as liquid flow passages between first channel 1442 of stationary disc 1436 and a pump chamber 1460 under membrane 1451.

When motor 1438 is rotating, membrane 1451 moves up and down. As membrane 1451 moves up and down, rotatable disc 1437 connects first channel 1442 alternately to second and third channels 1444 and 1445 so that liquid is either transmitted from second channel 1444 or third channel 1445 to pump chamber 1460 or received from pump chamber 1456 by second channel 1444 or third channel 1445. In FIG. 228D, first channel 1442 is shown as being connected to second channel via opened channel 1450 so that second channel 1444 receives liquid through first channel 1442 from chamber 1460.

The particular material selected for discs 1436 and 1437 is important because the selected material must be able to function using very fine tolerances without such discs sticking to one another over time. There are several materials available on the market that are suitable for this purpose, e.g. ceramic or ceramic mixed with other materials, such as carbon fiber.

FIG. 229 shows the medical device according to an embodiment in which the medical device is adapted to replace the contacting surface of the caput femur of the femoral bone of a human patient. The medical device according to this embodiment comprises an artificial contacting surface 1103b, further comprising a plurality of channels 1105 adapted to lubricate the hip joint of a human patient with a lubricating fluid. The medical device further comprises a fixating portion 44 for fixating the medical device to the caput femur and/or the collum femur of the femoral bone.

FIG. 230 shows the medical device according to FIG. 229 in section, showing the medical device comprising a plurality of channels in fluid connection with a reservoir (not shown) through a conduit 1106 placed centrally in the fixating portion 44, the channels 1105 being fully integrated in the medical device. The conduit 1106 transports lubricating fluid to the inlet 1123 for further distribution to the channels 1105. The conduit ends up in a connecting section 1111 which is adapted to connect the conduit to a second conduit 1106 or a reservoir, or additional channels.

FIG. 231 shows a frontal view of the body of a human patient, illustrating a laparoscopic/arthroscopic method of operating the hip joint to provide a medical device according to any of the embodiments herein from the opposite side from acetabulum 8. The hip joint comprises the acetabulum 8 and the caput femur 5. The small incisions 14 in the abdominal wall of the human patient allows the insertion of laparoscopic/arthroscopic trocars 33a,b,c into the body of the patients. Whereafter one or more camera 34, a surgical instrument adapted to create a hole in the pelvic bone 35, or instruments 36 for introducing, placing, connecting, attaching, creating or filling an implantable medical device, can be inserted into the body through said laparoscopic/arthroscopic trocars 33a,b,c.

FIG. 232 shows a lateral view of the body of a human patient, with the hip joint shown in section. The hip joint comprises a caput femur 5 placed at the very top of collum femur 6 which is the top part of the femur bone 7. The caput femur 5 is in connection with the acetabulum 8 which is a bowl shaped part of the pelvic bone 9. Laparoscopic/arthroscopic trocars 33a,b,c is being used to reach the hip joint 39 with one or more camera 34, a surgical instrument 35 adapted to create a hole in the pelvic bone 9, or instruments 36 for introducing, placing, connecting, attaching, creating or filling an implantable medical device.

FIG. 233 shows the creation of a hole 18 in the pelvic bone 9, after the pelvic bone 9 has been dissected. The hole 18 is created from the abdominal side of the pelvic bone 9 through repetitive or continuous movement of a hole creating device 22 placed into the human patient from the abdominal side of the pelvic bone 9. The hole 18 passes through the pelvic bone 9 from the opposite side from acetabulum 8 and into the hip joint 19. According to a first embodiment the hole 18 is large which allows an implantable medical device to pass through the hole 18 in its full functional size.

FIG. 234 shows a second embodiment in which the hole 20 created in a surgical or laparoscopic/arthroscopic method is much smaller as shown in FIG. 234 allowing the hole creating device 22 creating the hole 20 to be smaller, and thus also the incision and dissection performed in the human body.

FIG. 235 shows the hip joint in section when a medical device has been provided between the caput femur 5 and the acetabulum. The medical device according to this embodiment comprises multiple channels 1105 connected to a conduit 1106 which in turn is connected to a connecting portion placed in the hole in the pelvic bone 9. The conduit 1106 transports lubricating fluid to the inlet 1123 for further distribution to the channels 1105. For insertion through a hole 18 in the pelvic bone 9 being smaller than the medical device the medical device could be rolled or compressed, or according to another embodiment, moulded in place either in a mould adapted to be resorbed by the human body, melt or serve as the surface of the medical device. The medical device could be adapted to be fixated using adhesive or a mechanical fixating element.

FIG. 236a shows a hip joint in section when a medical device is being provided, through a hole 18 in the pelvic bone 9 for replacing the contacting surface of the caput femur 5. The medical device comprises an artificial contacting surface 1103b and a fixating portion 44 placed centrally in the medical device and adapted to fixate the medical device to the caput femur 5. The medical device comprises a plurality of channels 1105 which exits at the artificial contacting surface for lubricating the hip joint. The channels are in fluid connection with a conduit 1106 which in turn is connected to an interconnecting part 1111b adapted to connect the conduit to a second conduit 1106b or a second portion of the conduit 1106b, which in turn is in fluid connection with a reservoir 1108 placed in the femoral bone 7 of the human patient. The reservoir 1108 is placed in the femoral bone 7 and is adapted to hold a pressurized lubricating fluid, which according to the embodiment shown in FIG. 236a is pressurized by means of said reservoir 1108 being spring loaded by means of a spring 1109 in connection with a movable wall portion in the form of a piston 1110 pressurizing the lubricating fluid. The reservoir 1108 is furthermore connected to an injection port 1107 which is positioned in connection with the femoral bone 7 below the greater trochanter 1186, however, any other suitable placement is also conceivable, in connection with bone, in a cavity or subcutaneously. The medical device is according to the embodiment of FIG. 236a operable using a pressurized reservoir, however according to other embodiments the medical device is operable by a powered operating device, such as an implantable pump, which could be powered by direct propulsion, such as inductive or magnetic propulsion, or by an accumulated energy source, such as a battery. The channels or conduits could according to one embodiment (not shown) comprise a valve for closing the flow of lubricating fluid through the conduit 1106 or channel 1105, thereby closing the connection between the reservoir and the artificial contacting surface. The valve could be powered and adapted to be controlled form outside of the human body by means of for example a remote control.

FIG. 236b shows the hip joint in section when a medical device according to another embodiment has been provided to the hip joint, replacing the contacting surface of the caput femur. The medical device comprises an artificial contacting surface 1103b comprising a plurality of channels 1105 which are connected to a conduit 1106, 1106b placed in fixating part of the medical device. The conduit is in turn in fluid connection with a reservoir 1108 placed inside of the femoral bone, preferably in the cancellous parts of the femoral bone, the reservoir is thereby in fluid connection with the channels of the medical device for lubricating the artificial contacting surface 1103b of the medical device.

FIG. 237 shows the hip joint in section when an implantable medical device adapted to replace the acetabulum contacting surface is being provided. The medical device comprises an artificial acetabulum surface 65 comprising a plurality of channels connected to a conduit 1106 by an inter-connecting part 1111. The medical device is according to the embodiment shown in FIG. 237 adapted to be placed in a hole 18 in the pelvic bone 9 for replacing the acetabulum contacting surface 65. FIG. 237 furthermore shows a unit to which the conduit 1106 is connected, according to one embodiment the unit comprises a reservoir 1108 and two pressure creating devices 1113a, 1113b adapted to create a pressure for pressurizing the lubricating fluid for pressing said lubricating fluid through the conduit 1106 and further through the plurality of channels 1105 for lubricating the implantable medical device. The conduit 1106 transports lubricating fluid to the inlet 1123 for further distribution to the channels 1105.

The pressure creating devices could be spring loaded or comprise of a pressurized gas filled element which is further pressurized by the injecting of a lubricating fluid into the reservoir 1108. The unit further comprises an injection port 1107 which comprises a self-sealing membrane 1112, which preferably is a Parylene coated silicone membrane. According to another embodiment the unit comprises a powered operation device such as a pump housed in the pressure creating device 1113a which pumps the lubricating fluid from the reservoir 1108 through the conduit 1106 to the plurality of channels 1105. According to one embodiment the pump is powered by a battery housed in the pressure creating device 1113b.

FIG. 238a shows a surgical instrument adapted to insert a medical device according to any of the embodiments herein, or a mould for creating a medical device, according to a first embodiment. The surgical instrument comprises a gripping portion 76 and a handling portion 77. According to the embodiments shown in FIG. 238a,b,c the instrument further comprises a rotation element 78 that enables the gripping part 76 to rotate in relation to the handling part 77, however it is equally conceivable that the surgical instrument lacks this rotation element 78.

FIG. 238b shows the surgical instrument adapted to insert a prosthesis, prosthetic parts or parts needed to create or provide a hip joint surface, according to a second embodiment. According to this embodiment the surgical instrument further comprises a parallel displaced section 79, which increases the reach of the instrument and facilitates the reaching of the hip joint through a hole in the pelvic bone from the opposite side from acetabulum.

FIG. 238c shows the surgical instrument adapted to insert a prosthesis, prosthetic parts or parts needed to create or provide a hip Joint surface, according to a third embodiment. According to this embodiment the surgical instrument further comprises two angle adjusting members 80a,b. The angle adjusting members could be adjustable for varying the angle of said gripping part 76 in relation to the handling portion 77, or fixed in an angle suitable for creating operating in a hip joint through a hole in the pelvic bone from the opposite side from acetabulum 8.

FIG. 239 shows the hip joint in section when a medical device has been provided. The implantable medical device is adapted to replace the acetabulum surface and is inserted through a hole 18 in the pelvic bone 9, however, in other embodiments it is equally conceivable that the medical device is adapted to be inserted through a hole in the femoral 7 bone or the hip joint capsule. The medical device comprises a plurality of channels 1105 interconnected through a conduit 1106 which places the channels 1105 in fluid connection with each other. The conduit 1106 transports lubricating fluid to the inlet 1123 for further distribution to the channels 1105. The conduit 1106 is further connected to a first portion of an interconnecting part 1111a which is adapted to be connected to a second portion of an interconnecting part 1111b. The interconnecting part 1111 connects a first portion of the conduit 1106 to a second portion of the conduit 1106, enabling a first portion of the conduit 1106 to be inserted from the acetabulum side of the pelvic bone 9 and a second portion of the conduit 1106 to be inserted from the pelvic side, or opposite acetabulum side of the pelvic bone 9. The connection of two portions of the conduit 1106 is particularly beneficial when the medical device has been inserted through a hole 18 in the femoral bone 7 or the hip joint capsule and the reservoir 1108 is implanted in the abdominal region of the human patient, or in another area on the abdominal side of the pelvic bone 9. The conduit 1106 is then further connected to the reservoir 1108 and adapted to transport a lubricating fluid from the reservoir 1108 to an area of the hip joint. The reservoir 1108 is according to the embodiment shown in FIG. 239 adapted to place the lubricating fluid under pressure by means of a spring 1109 exerting a force on a movable wall portion in the form of a piston 1110 pressing the lubricating fluid through the conduit 1106 and further through the channels 1105. The reservoir 1108 further comprises an injection port 1107 placed in the top part of the reservoir 1108 for refilling the reservoir 1108 and in the same event increasing the pressure of the lubricating fluid.

FIG. 240a shows a human patient in a lateral view showing the hip joint in section. The femoral bone 7 has a proximal part comprising the collum femur 6 and most proximal the caput femur 5. In FIG. 240a a hole 82 is being created from an incision made in the thigh, the hole travels into the femoral bone 7, following the collum femur 6 and exiting through the caput femur 5 and thus into the hip joint. The hole is used to provide the hip joint with a medical device which preferably is possible to roll or bend for insertion through said hole 82.

FIG. 240b shows the hip joint in section when the medical device has been provided through the hole 82 in the femoral bone 7 and fixated in the acetabulum bowl 8. The medical device comprises a plurality of channels 1105 connected to each other by a conduit 1106. According to other embodiments, the medical device could be provided through the hip joint capsule, or a hole in the pelvic bone 9. After the medical device has been provided, a tool 1180 housing a reservoir 1108 connected to a conduit 1106′ is used to provide the reservoir 1108 to the hole 82 in the femoral bone 7 and to connect the reservoir to the conduit 1106 of the medical device.

FIG. 240c shows the hip joint in section when the reservoir 1108, placed in the hole 82 in the femoral bone 7 has been connected to the medical device. Furthermore a conduit 1106′ reaching from the reservoir 1108 to an injection port 1107 for refilling and/or pressurizing the reservoir 1108.

FIG. 240d shows the reservoir unit in further detail, the reservoir unit comprises an inter-connecting portion 1111 placed at the end part of the reservoir unit, a pressurized reservoir 1108, which according to the embodiment of FIG. 240d is pressurized by means of a spring 1109 pushing a movable wall portion 1110 in the form of a piston 1110. The reservoir unit further comprises a conduit 1106′ in connection with the reservoir, and in connection with an injection port 1107, for filling the and/or pressurizing the reservoir 1108 comprising the lubricating fluid. The injection port 1107 comprises a self-sealing membrane, which could be a self-sealing Parylene coated silicone membrane, to inhibit cell migration on the surface of the injection port. The section A-A shows the centrally placed conduit 1106 in the center of the reservoir 1108 for filling and/or pressurizing the reservoir 1108.

FIG. 241 shows a lateral view of a human patient in section, when a lubricating fluid is being injected into an injection port 1107, by means of an injecting member 92 comprising a container 1115 adapted to contain the lubricating fluid to be injected. The injection port is connected to an implantable medical device placed in the hip joint through a conduit 1106 adapted to supply the fluid connection between the injection port and the medical device. The medical device in turn comprises a plurality of channels 1105 for lubricating the artificial contacting surfaces and thereby lubricating the hip joint.

According to the embodiment shown in FIG. 241 the medical device has been supplied from the abdominal side of the pelvic bone 9 through a hole made in the pelvic bone which afterwards has been refilled with the removed bone plug and sealed and fixated with a mechanical fixating part attached with screws. According to other embodiments the medical device is provided from the hip joint side of the pelvic bone 9 through the hip joint capsule 12 or the femoral bone 7 and thereafter connected to the conduit 1106 on the abdominal side of the pelvic bone 9 through an interconnecting part 1111. This enables the placing of the injection port 1107 in the abdominal region, subcutaneously, in a cavity and/or supported by the muscular or fascia tissue.

FIG. 242 shows the medical device in an opposite embodiment where the medical device comprises a first artificial contacting surface 112 comprising a convex shape towards a center of the hip joint. The first artificial contacting surface 112 is adapted to be fixated to the pelvic bone 9 of the human patient. The artificial convex hip joint surface 112 is adapted to be fixated to the pelvic bone 9, and is adapted to be inserted through a hole 18 in the pelvic bone 9. The medical device comprises a nut 120, comprising threads for securely fixating the medical device to the pelvic bone 9. The medical device further comprises a prosthetic part 118 adapted to occupy the hole 18 created in the pelvic bone 9 after the medical device has been implanted in the patient. The prosthetic part 118 comprises supporting members 119 adapted to be in contact with the pelvic bone 9 and assist in the carrying of the load placed on the medical device from the weight of the human patient in normal use. Normal use is defined as the same as a person would use a natural hip joint. Further the medical device comprises a locking element 116 comprising a surface 117 adapted to be in contact with the artificial convex hip joint surface 112.

The locking element 116 further comprises fixating members 115 which are adapted to assist in the fixation of the locking member 116 to the caput femur 5 or collum femur 6, which in turns fixates the artificial convex hip joint surface 112. The artificial convex hip joint surface 112 is fixated to an attachment rod 113 comprising a thread 114 that corresponds to the thread of the nut 120 in connection with the prosthetic part 118. The medical device comprises a plurality of channels 1105 adapted to lubricate the artificial contacting surface 112. The plurality of channels 1105 are connected to each other through a conduit 1106 adapted to transport a lubricating fluid from a reservoir 1108 to the plurality of channels 1105 which are fully integrated in the artificial contacting surface 112 of the medical device for lubricating the artificial contacting surface 112 and thereby lubricating the hip joint.

FIG. 243 shows the medical device according to FIG. 242 when said medical device is placed inside of the hip joint. The first artificial contacting surface 112 comprising a convex shape towards a center of the hip joint is positioned in a second artificial contacting surface 109 comprising a concave shape towards the center of the hip joint. The second artificial contacting surface 109 is placed and fixated in the caput 5 and collum femur 6 of the femoral bone and secured by a locking element 116 comprising a surface 117 facing the first artificial convex contacting surface 112. The medical device comprises a plurality of channels 1105 which are connected to a conduit 1106 placed centrally in the medical device for providing a lubricating fluid to the medical device and lubricate the artificial contacting surface 112 and thereby the hip joint.

FIG. 244 shows the providing of a prosthetic part 118 to the hole 18 in the pelvic bone 9. The prosthetic part 118 comprises supporting members 119 adapted to be in contact with the pelvic bone 9 and assist in the carrying of the load placed on the medical device from the weight of the human patient in normal use.

FIG. 245 shows an alternative embodiment of the medical device in the opposite embodiment in which the part of the medical device comprising an artificial concave hip joint surface placed in the caput and collum femur 6 comprises a plurality of lubricating channels 1105 which are connected to a conduit 1106b establishing a fluid connection between the medical device and the reservoir located in the cancellous bone of the collum femur 6. The reservoir is adapted to be refilled through an injection port 1107 which according to the embodiment of FIG. 245 is placed in connection with the femoral bone 7 and situated below the greater trochanter 1186. The reservoir unit, and the function thereof, is described in further detail with reference to FIGS. 236a and 236b. FIG. 245 furthermore shows the prosthetic part 118, when fixated to the pelvic bone 9 using screws 121. The screws could be assisted or replaced by an adhesive which could be applied in connection to the screws or at the surface S between the prosthetic part and the pelvic bone 9.

FIG. 246 shows the right leg of a human patient. The femoral bone 102 having a distal part comprising the lateral condyle 105, the medial condyle 106 and an area between said lateral and said medial condyle. The sections of the distal part of the femoral bone 102 comprise contacting surfaces of the knee joint. The knee joint furthermore comprises the patella 101, which is a triangular bone which articulates with the femur 102 and covers and protects the knee joint. The knee joint also comprises the menisci 107, 108 which are cartilaginous elements within the knee joint which serve as articulating surfaces to protect the ends of the bones from rubbing on each other. The menisci 107, 108 also acts as shock absorbers in the knee joint, to absorb the shocks from the movement of the human patient. There are two menisci 107, 108 in each knee, the medial meniscus 107 and the lateral meniscus 108. In patients with osteoarthritis the menisci 107, 108 which acts as articulating surfaces i.e. weight carrying surfaces are worn away and, in extreme cases, bone can be exposed in the joint. The knee joint is protected by the knee joint capsule also known as the articular capsule of the knee joint or the capsular ligaments of the knee joint. The knee joint capsule is wide and lax; thin in front and at the side; and contains the patella 101, ligaments, menisci 107, 108, and burse, which are small fluid-filled sacs made of white fibrous tissue. The knee joint capsule consists of a synovial and a fibrous membrane separated by fatty deposits anteriorly and posteriorly.

FIG. 247 shows the knee joint when artificial knee joint surfaces 130, 116a has been provided to the distal part of the femoral bone 102 and the proximal part of the tibia bone 104. A lateral and medial channel 125a,b supplies the contacting surfaces and thereby the knee joint with lubricating fluid for reducing the friction of the knee joint.

FIG. 248 shows the body of a human patient in a frontal view where a reservoir unit 127 is implanted subcutaneously in the abdominal region of the human patient. The reservoir unit according to this embodiment comprises an operating device in the form of a pump 1130 which is powered by a battery 128 for pumping a fluid from the reservoir 129 through a conduit to a channel 125 supplying the artificial contacting surfaces of the knee joint with a lubricating fluid. The reservoir unit is fixated to the muscular or fascia tissue 1181 of the abdominal wall through the muscular or fascia 1181 tissue being clamped between the reservoir unit and the injection port 1107 arranged at the outside of the muscular or fascia tissue 1181.

FIG. 249a shows an embodiment where the medical device comprises an artificial knee joint 1115a surface clamps the medial 106, lateral 105 or both the medial and lateral condyle 106, 105 of the knee joint, being the distal portion of the femoral bone 7. The medical device, according to this embodiment comprises a plurality of channels 1105 for lubricating the artificial contacting surfaces, the plurality of channels is in fluid connection with each other through a conduit 1106 which in turn is in fluid connection with a reservoir 1108 comprising an injection port 1107 for refilling the reservoir or pressurizing the lubricating fluid contained in said reservoir 1108. The conduit 1106 transports lubricating fluid to the inlet 1123 for further distribution to the channels 1105.

FIG. 249b shows the knee joint in a lateral view when a medical device comprising a second artificial contacting surface 1102 has been provided to the proximal part of the tibia bone 104, which together with the fibula bone 103 makes up the lower part of the leg. The artificial knee joint surface comprises a plurality of channels 1105 which are in fluid connection with a conduit 1106 adapted to transport lubricating fluid from a reservoir 1108. The reservoir 1108 is according to the embodiment of FIG. 249b placed at the rear side of the tibia bone 104 and fixated to the tibia bone 104 and comprises an injection port 1107 for injecting a lubricating fluid into the reservoir 1108 and/or pressurizing a lubricating fluid contained in the reservoir 1108. The conduit 1106 transports lubricating fluid to the inlet 1123 for further distribution to the channels 1105.

FIG. 250 shows the medical device for implantation in a knee joint in further detail. The medical device comprises a plurality of channels 1105 placed along the artificial contacting surface of the medical device, for lubricating the contacting surface of the medical device. The channels 1105 are connected to a conduit 1106 for transport of the lubricating fluid along the artificial contacting surface 1101 of the medical device. The conduit 1106 transports lubricating fluid to the inlets 1123 for further distribution to the channels 1105.

FIG. 251 shows a sectional side-view of the medical device displaying the channels 1105 being fully integrated in the artificial contacting surface and connected to each other, the conduit 1106 supplies the 1105 channels with lubricating fluid for lubricating the artificial contacting surface of the medical device. The conduit 1106 transports lubricating fluid to the inlet 1123 for further distribution to the channels 1105.

FIG. 252 shows a medical device for implantation in a knee joint of a human patient, the medical device comprises a several medical device parts 1119 adapted to be connected to each other and to a medical device base part 1118 by means of mechanical fixation elements 1120 supplying a form fitting between the plurality of medical device parts 1119 and the base part 1118. The medical device base part 1118 furthermore comprises a fixation portion 1117 which is adapted to supply mechanical fixation of the medical device to a human bone, such as the proximal part of the tibia bone. The medical device base part 1118 furthermore comprises a channel for supplying a lubricating fluid to the artificial contacting surface of the knee joint.

FIG. 253 shows the medical device according to FIG. 252, when assembled.

FIG. 256 shows the medical device according to FIGS. 252 and 253 when the medical device is being fixated to the tibia bone 104.

FIG. 255 shows the proximal part of the tibia bone when a medical device comprising an artificial contacting surface 1116 has been fixated to the tibia bone 104. The channel 1105 of the artificial contacting surface is connected to a conduit 1106 which supplies a fluid connection between the channel 1105 of the medical device and a first and second reservoir 1108 placed inside of the tibia bone 1104 on the medial and lateral side. The conduit further connects the first and second reservoir to an injection port 1107 placed on the medial side of the pelvic bone for refilling and/or pressurizing the reservoirs 1108.

The reservoirs 1108, according to the embodiment shown in FIG. 255 are adapted to place the lubricating fluid under pressure, thereby pressing the lubricating fluid out of the channels 1105 onto the artificial contacting surface 1116, for lubricating the knee joint. For this purpose, the reservoir 1108 comprises a spring 1109 which is in connection with a movable wall portion in the form of a piston 1110, for pressing the lubricating fluid.

FIG. 256 shows the human patient in a frontal view when an implantable lubrication system 1120a has been implanted. The implantable lubrication system 1120a is adapted to inject a lubricating fluid continuously, intermittently or when needed into said hip joint. According to the embodiment shown in FIG. 256 the implantable lubricating system comprises two interconnected units 1121, 1122. The two interconnected units are placed in the abdominal region of the human patient and is in connection with the hip joint through a conduit 1106.

FIG. 257 shows the implantable lubricating system 1120a, which could be used in combination with any of the medical devices described herein, in further detail. According to the embodiment shown, the implantable lubricating system comprises a first unit 1121 comprising a pumping member 123 adapted to pump the lubricating fluid from a reservoir 1108 to an area of the hip joint. The first unit 1121 furthermore comprises an injection port 1107 for filling the reservoir 1108 from outside of the human body without having to perform a surgical procedure. The injection port 1107 comprises a self-sealing membrane which is penetrable with a needle attached to a syringe. The first unit 1121 further comprises a receiver of wireless energy 124 preferably comprising a coil. Said receiver of wireless energy is used to charge a battery 126. According to this embodiment the implantable lubrication system 1120a further comprises a second unit 1122 which in turn comprises a battery 126 and a fluid reservoir 1108. The lubricating fluid 128 is pumped from the reservoir 1108, through the first unit 1121 with the pumping device, through the conduit 1106 and into the area of the hip joint where it helps lubricating the hip joint surfaces or the artificial contacting surfaces of the implantable medical device. The lubricating fluid is preferably a biocompatible lubricating fluid such as hyaluronic acid.

FIG. 258 shows the implantable lubricating system adapted to be used with any of the medical device herein, according to an embodiment wherein the implantable lubricating system is a circulating lubricating system comprising one inlet 1340 into the joint to be lubricated and one outlet 131. Preferably this system is a system for continuous lubrication where the pumping member 123 continuously circulates the lubricating fluid 128 inside of the hip joint.

FIG. 259 shows an implantable lubricating system for circulating lubrication adapted to be used with any of the medical device herein, wherein the lubricating system further comprises a filtering member 132 for filtering the lubricating fluid. The filter is adapted to be self-cleaning and the out filtered matter is disposed through the disposal channel 133, either into the abdomen of the human patient, or into a container attached to the disposal channel 133. Through the filtering of the lubricating fluid 128 the circulating lubricating system can operate for long periods without the need of any surgical procedures.

FIG. 260 shows the lubricating fluid of FIG. 257, when lubricating an implantable medical device comprising an artificial contacting surface 45 by providing a lubricating fluid 128.

FIG. 261 shows a lubricating system, which could be adapted to be used in combination with any of the medical devices 100 herein, according to another embodiment wherein the lubricating system comprises a unit 1310 comprising a retractable needle 1311 fixated to an operating system for operating said retractable needle 1311. The needle is adapted to penetrate a self-sealing membrane 1314 placed in the pelvic bone 9 for injecting a lubricating fluid into the hip joint. A conduit 1106 is adapted to supply the unit 1310 with a lubricating fluid from an injection port and/or from an additional reservoir which could be implanted subcutaneously or in a cavity of the body.

FIG. 262 shows the lubricating system in a state in which the retractable needle 1311 is in its advanced position by the operating device having operated the retractable needle 1311. The needle thereby penetrates the self-sealing membrane 1314 and is placed in a position in which injection of a lubricating fluid is possible.

FIG. 666 illustrates a system for treating a disease comprising an apparatus 10 of the present invention placed in the abdomen of a patient. An implanted energy-transforming device 1002 is adapted to supply energy consuming components of the apparatus with energy via a power supply line 1003. An external energy-transmission device 200 for non-invasively energizing the apparatus 10 transmits energy by at least one wireless energy signal. The implanted energy-transforming device 1002 transforms energy from the wireless energy signal into electric energy which is supplied via the power supply line 1003.

The implanted energy-transforming device 1002 may also comprise other components, such as: a coil for reception and/or transmission of signals and energy, an antenna for reception and/or transmission of signals, a microcontroller, a charge control unit, optionally comprising an energy storage, such as a capacitor, one or more sensors, such as temperature sensor, pressure sensor, position sensor, motion sensor etc., a transceiver, a motor, optionally including a motor controller, a pump, and other parts for controlling the operation of a medical implant.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field.

The wireless energy-transmission device 200 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal.

Generally speaking, the energy-transforming device 1002 is provided for transforming wireless energy of a first form transmitted by the energy-transmission device 200 into energy of a second form, which typically is different from the energy of the first form. The implanted apparatus 10 is operable in response to the energy of the second form. The energy-transforming device 1002 may directly power the apparatus with the second form energy, as the energy-transforming device 1002 transforms the first form energy transmitted by the energy-transmission device 200 into the second form energy. The system may further include an implantable accumulator, wherein the second form energy is used at least partly to charge the accumulator.

Alternatively, the wireless energy transmitted by the energy-transmission device 200 may be used to directly power the apparatus, as the wireless energy is being transmitted by the energy-transmission device 200. Where the system comprises an operation device for operating the apparatus, as will be described below, the wireless energy transmitted by the energy-transmission device 200 may be used to directly power the operation device to create kinetic energy for the operation of the apparatus.

The wireless energy of the first form may comprise sound waves and the energy-transforming device 1002 may include a piezo-electric element for transforming the sound waves into electric energy.

The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus.

Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus.

The external energy-transmission device 200 also includes a wireless remote control having an external signal transmitter for transmitting a wireless control signal for non-invasively controlling the apparatus. The control signal is received by an implanted signal receiver which may be incorporated in the implanted energy-transforming device 1002 or be separate there from.

The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field.

The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals.

FIG. 667 illustrates the system of FIG. 666 in the form of a more generalized block diagram showing the apparatus 10, the energy-transforming device 1002 powering the apparatus 10 via power supply line 1003, and the external energy-transmission device 200, The patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 1006 operable for example by polarized energy also is implanted in the patient for reversing the apparatus 10. When the switch is operated by polarized energy the wireless remote control of the external energy-transmission device 200 transmits a wireless signal that carries polarized energy and the implanted energy-transforming device 1002 transforms the wireless polarized energy into a polarized current for operating the electric switch 1006. When the polarity of the current is shifted by the implanted energy-transforming device 1002 the electric switch 1006 reverses the function performed by the apparatus 10.

FIG. 669 shows an embodiment identical to that of FIG. 667, except that an operation device 1007 implanted in the patient for operating the apparatus 10 is provided between the implanted energy-transforming device 1002 and the apparatus 10. This operation device can be in the form of a motor 1007, such as an electric servomotor. The motor 1007 is powered with energy from the implanted energy-transforming device 1002, as the remote control of the external energy-transmission device 200 transmits a wireless signal to the receiver of the implanted energy-transforming device 1002.

FIG. 670 shows an embodiment identical to that of FIG. 667, except that it also comprises an operation device in the form of an assembly 1008 including a motor/pump unit 1009 and a fluid reservoir 1010 is implanted in the patient. In this case the apparatus 10 is hydraulically operated, i.e. hydraulic fluid is pumped by the motor/pump unit 1009 from the fluid reservoir 1010 through a conduit 1011 to the apparatus 10 to operate the apparatus, and hydraulic fluid is pumped by the motor/pump unit 1009 back from the apparatus 10 to the fluid reservoir 1010 to return the apparatus to a starting position. The implanted energy-transforming device 1002 transforms wireless energy into a current, for example a polarized current, for powering the motor/pump unit 1009 via an electric power supply line 1012.

Instead of a hydraulically operated apparatus 10, it is also envisaged that the operation device comprises a pneumatic operation device. In this case, the hydraulic fluid can be pressurized air to be used for regulation and the fluid reservoir is replaced by an air chamber.

In all of these embodiments the energy-transforming device 1002 may include a rechargeable accumulator like a battery or a capacitor to be charged by the wireless energy and supplies energy for any energy consuming part of the system.

As an alternative, the wireless remote control described above may be replaced by manual control of any implanted part to make contact with by the patient's hand most likely indirect, for example a press button placed under the skin.

FIG. 671 shows an embodiment comprising the external energy-transmission device 200 with its wireless remote control, the apparatus 10, in this case hydraulically operated, and the implanted energy-transforming device 1002, and further comprising a hydraulic fluid reservoir 1013, a motor/pump unit 1009 and a reversing device in the form of a hydraulic valve shifting device 1014, all implanted in the patient. Of course, the hydraulic operation could easily be performed by just changing the pumping direction and the hydraulic valve may therefore be omitted. The remote control may be a device separated from the external energy-transmission device or included in the same. The motor of the motor/pump unit 1009 is an electric motor. In response to a control signal from the wireless remote control of the external energy-transmission device 200, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 10. The remote control of the external energy-transmission device 200 controls the hydraulic valve shifting device 1014 to shift the hydraulic fluid flow direction between one direction in which the fluid is pumped by the motor/pump unit 1009 from the hydraulic fluid reservoir 1013 to the apparatus 10 to operate the apparatus, and another opposite direction in which the fluid is pumped by the motor/pump unit 1009 back from the apparatus 10 to the hydraulic fluid reservoir 1013 to return the apparatus to a starting position.

FIG. 672 shows an embodiment comprising the external energy-transmission device 200 with its wireless remote control, the apparatus 10, the implanted energy-transforming device 1002, an implanted internal control unit 102 controlled by the wireless remote control of the external energy-transmission device 200, an implanted accumulator 1016 and an implanted capacitor 1017. The internal control unit 102 arranges storage of electric energy received from the implanted energy-transforming device 1002 in the accumulator 1016, which supplies energy to the apparatus 10. In response to a control signal from the wireless remote control of the external energy-transmission device 200, the internal control unit 102 either releases electric energy from the accumulator 1016 and transfers the released energy via power lines 1018 and 1019, or directly transfers electric energy from the implanted energy-transforming device 1002 via a power line 1020, the capacitor 1017, which stabilizes the electric current, a power line 1021 and the power line 1019, for the operation of the apparatus 10.

The internal control unit is preferably programmable from outside the patient's body. In a preferred embodiment, the internal control unit is programmed to regulate the apparatus 10 according to a pre-programmed time-schedule or to input from any sensor sensing any possible physical parameter of the patient or any functional parameter of the system.

In accordance with an alternative, the capacitor 1017 in the embodiment of FIG. 672, 10 may be omitted. In accordance with another alternative, the accumulator 1016 in this embodiment may be omitted.

FIG. 673 shows an embodiment identical to that of FIG. 667, except that a battery 1022 for supplying energy for the operation of the apparatus 10 and an electric switch 1023 for switching the operation of the apparatus 10 also are implanted in the patient. The electric switch 1023 may be controlled by the remote control and may also be operated by the energy supplied by the implanted energy-transforming device 1002 to switch from an off mode, in which the battery 1022 is not in use, to an on mode, in which the battery 1022 supplies energy for the operation of the apparatus 10.

FIG. 674 shows an embodiment identical to that of FIG. 673, except that an internal control unit 102 controllable by the wireless remote control of the external energy-transmission device 200 also is implanted in the patient. In this case, the electric switch 1023 is operated by the energy supplied by the implanted energy-transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the apparatus 10.

FIG. 675 shows an embodiment identical to that of FIG. 674, except that an accumulator 1016 is substituted for the battery 1022 and the implanted components are interconnected differently. In this case, the accumulator 1016 stores energy from the implanted energy-transforming device 1002. In response to a control signal from the wireless remote control of the external energy-transmission device 200, the internal control unit 102 controls the electric switch 1023 to switch from an off mode, in which the accumulator 1016 is not in use, to an on mode, in which the accumulator 1016 supplies energy for the operation of the apparatus 10. The accumulator may be combined with or replaced by a capacitor.

FIG. 676 shows an embodiment identical to that of FIG. 675, except that a battery 1022 also is implanted in the patient and the implanted components are interconnected differently. In response to a control signal from the wireless remote control of the external energy-transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the apparatus 10.

Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the apparatus 10.

It should be understood that the switch 1023 and all other switches in this application should be interpreted in its broadest embodiment. This means a transistor, MCU, MCPU, ASIC, FPGA or a DA converter or any other electronic component or circuit that may switch the power on and off. Preferably the switch is controlled from outside the body, or alternatively by an implanted internal control unit.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the apparatus 10 (mechanically operated). Even simpler is to switch the direction of the motor electronically. The gear box interpreted in its broadest embodiment may stand for a servo arrangement saving force for the operation device in favor of longer stroke to act.

FIG. 678 shows an embodiment identical to that of FIG. 684 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the apparatus 10.

FIG. 679 schematically shows conceivable combinations of implanted components of the apparatus for achieving various communication options. Basically, there are the apparatus 10, the internal control unit 102, motor or pump unit 1009, and the external energy-transmission device 200 including the external wireless remote control. As already described above the wireless remote control transmits a control signal which is received by the internal control unit 102, which in turn controls the various implanted components of the apparatus.

A feedback device, preferably comprising a sensor or measuring device 1025, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow.

The feedback may be sent to the internal control unit or out to an external control unit preferably via the internal control unit. Feedback may be sent out from the body via the energy transfer system or a separate communication system with receiver and transmitters.

The internal control unit 102, or alternatively the external wireless remote control of the external energy-transmission device 200, may control the apparatus 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver. Alternatively, the wireless remote control may comprise a signal receiver or transceiver and the internal control unit 102 may comprise a signal transmitter or transceiver. The above transceivers, transmitters and receivers may be used for sending information or data related to the apparatus 10 from inside the patient's body to the outside thereof.

Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly.

FIG. 680 shows an alternative embodiment wherein the apparatus 10 is regulated from outside the patient's body. The system 100 comprises a battery 1022 connected to the apparatus 10 via a subcutaneous electric switch 1026. Thus, the regulation of the apparatus 10 is performed non-invasively by manually pressing the subcutaneous switch, whereby the operation of the apparatus 10 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 681 shows an alternative embodiment, wherein the system 100 comprises a hydraulic fluid reservoir 1013 hydraulically connected to the apparatus. Non-invasive regulation is performed by manually pressing the hydraulic reservoir connected to the apparatus. Alternatively, the hydraulic fluid reservoir 1013 is adapted to work with an injection port for the injection of hydraulic fluid, preferably for calibration of hydraulic fluid.

The system may include an external data communicator and an implantable internal data communicator communicating with the external data communicator. The internal communicator feeds data related to the apparatus or the patient to the external data communicator and/or the external data communicator feeds data to the internal data communicator.

FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 1002 connected to implanted energy consuming components of the apparatus 10. Such an energy receiver 1002 may include an energy source and/or an energy-transforming device. Briefly described, wireless energy is transmitted from an external energy source 200a located outside the patient and is received by the internal energy receiver 1002 located inside the patient. The internal energy receiver is adapted to directly or indirectly supply received energy to the energy consuming components of the apparatus 10 via a switch 1026. An energy balance is determined between the energy received by the internal energy receiver 1002 and the energy used for the apparatus 10, and the transmission of wireless energy is then controlled based on the determined energy balance. The energy balance thus provides an accurate indication of the correct amount of energy needed, which is sufficient to operate the apparatus 10 properly, but without causing undue temperature rise.

In FIG. 682 the patient's skin is indicated by a vertical line SK. Here, the energy receiver comprises an energy-transforming device 1002 located inside the patient, preferably just beneath the patient's skin SK. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy-source 200a provided in an external energy-transmission device 200 located outside the patient's skin SK in the vicinity of the implanted energy-transforming device 1002.

As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used.

The amount of energy received by the implanted energy receiver may be compared with the energy used by the implanted components of the apparatus. The term “energy used” is then understood to include also energy stored by implanted components of the apparatus. A control device includes an external control unit 200b that controls the external energy source 200a based on the determined energy balance to regulate the amount of transferred energy. In order to transfer the correct amount of energy, the energy balance and the required amount of energy is determined by means of a determination device including an implanted internal control unit 102 connected between the switch 1026 and the apparatus 10. The internal control unit 102 may thus be arranged to receive various measurements obtained by suitable sensors or the like, not shown, measuring certain characteristics of the apparatus 10, somehow reflecting the required amount of energy needed for proper operation of the apparatus 10. Moreover, the current condition of the patient may also be detected by means of suitable measuring devices or sensors, in order to provide parameters reflecting the patient's condition. Hence, such characteristics and/or parameters may be related to the current state of the apparatus 10, such as power consumption, operational mode and temperature, as well as the patient's condition reflected by parameters such as; body temperature, blood pressure, heartbeats and breathing. Other kinds of physical parameters of the patient and functional parameters of the device are described elsewhere.

Furthermore, an energy source in the form of an accumulator 1016 may optionally be connected to the implanted energy-transforming device 1002 via the control unit 102 for accumulating received energy for later use by the apparatus 10. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 10, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 of the determination device is adapted to determine the energy balance and/or the currently required amount of energy, (either energy per time unit or accumulated energy) based on measurements made by the above-mentioned sensors or measuring devices of the apparatus 10, or the patient, or an implanted energy source if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal.

Alternatively, the determination device may include the external control unit 200b. In this alternative, sensor measurements can be transmitted directly to the external control unit 200b wherein the energy balance and/or the currently required amount of energy can be determined by the external control unit 200b, thus integrating the above-described function of the internal control unit 102 in the external control unit 200b. In that case, the internal control unit 102 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements.

Hence, the present solution according to the arrangement of FIG. 682 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus.

The internal signal transmitter 1027 and the external signal receiver 200c may be implemented as separate units using suitable signal transfer means, such as radio, IR (Infrared) or ultrasonic signals. Alternatively, the internal signal transmitter 1027 and the external signal receiver 200c may be integrated in the implanted energy-transforming device 1002 and the external energy source 200a, respectively, so as to convey control signals in a reverse direction relative to the energy transfer, basically using the same transmission technique. The control signals may be modulated with respect to frequency, phase or amplitude.

Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off In implementing this system in the arrangement of FIG. 682, the switch 1026 is either separate and controlled by the internal control unit 102, or integrated in the internal control unit 102. It should be understood that the switch 1026 should be interpreted in its broadest embodiment. This means a transistor, MCU, MCPU, ASIC FPGA or a DA converter or any other electronic component or circuit that may switch the power on and off.

To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 1027 to the external signal receiver 200c. Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized.

This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor.

With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 1026 is interconnected between the external energy source 200a and an operation device, such as an electric motor 1007 operating the apparatus 10. An external control unit 200b controls the operation of the external switch 1026 to effect proper operation of the apparatus 10.

FIG. 684 illustrates different embodiments for how received energy can be supplied to and used by the apparatus 10. Similar to the example of FIG. 682, an internal energy receiver 1002 receives wireless energy E from an external energy source 200a which is controlled by a transmission control unit 200b. The internal energy receiver 1002 may comprise a constant voltage circuit, indicated as a dashed box “constant V” in the figure, for supplying energy at constant voltage to the apparatus 10. The internal energy receiver 1002 may further comprise a constant current circuit, indicated as a dashed box “constant C” in the figure, for supplying energy at constant current to the apparatus 10.

The apparatus 10 comprises an energy consuming part 10a, which may be a motor, pump, restriction device, or any other medical appliance that requires energy for its electrical operation. The apparatus 10 may further comprise an energy storage device 10b for storing energy supplied from the internal energy receiver 1002. Thus, the supplied energy may be directly consumed by the energy consuming part 10a, or stored by the energy storage device 10b, or the supplied energy may be partly consumed and partly stored. The apparatus 10 may further comprise an energy stabilizing unit 10c for stabilizing the energy supplied from the internal energy receiver 1002. Thus, the energy may be supplied in a fluctuating manner such that it may be necessary to stabilize the energy before consumed or stored.

The energy supplied from the internal energy receiver 1002 may further be accumulated and/or stabilized by a separate energy stabilizing unit 1028 located outside the apparatus 10, before being consumed and/or stored by the apparatus 10. Alternatively, the energy stabilizing unit 1028 may be integrated in the internal energy receiver 1002. In either case, the energy stabilizing unit 1028 may comprise a constant voltage circuit and/or a constant current circuit.

It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope of the present invention.

FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window.

The schematic FIG. 685 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 668; the transmitting parts of the system are excluded.

The implementation of the general concept of energy balance and the way the information is transmitted to the external energy transmitter can of course be implemented in numerous different ways.

The schematic FIG. 685 and the above described method of evaluating and transmitting the information should only be regarded as examples of how to implement the control system.

Circuit Details In FIG. 685 the symbols Y1, Y2, Y3 and so on symbolize test points within the circuit. The components in the diagram and their respective values are values that work in this particular implementation which of course is only one of an infinite number of possible design solutions.

Energy to power the circuit is received by the energy receiving coil L1. Energy to implanted components is transmitted in this particular case at a frequency of 25 kHz. The energy balance output signal is present at test point Y1.

Those skilled in the art will realize that the above various embodiments of the system could be combined in many different ways. For example, the electric switch 1006 of FIG. 668 could be incorporated in any of the embodiments of FIGS. 671-677, the hydraulic valve shifting device 1014 of

FIG. 671 could be incorporated in the embodiment of FIG. 670, and the gear box 1024 could be incorporated in the embodiment of FIG. 669. Please observe that the switch simply could mean any electronic circuit or component.

The embodiments described in connection with FIGS. 682, 684 and 685 identify a method and a system for controlling transmission of wireless energy to implanted energy consuming components of an electrically operable apparatus. Such a method and system will be defined in general terms in the following.

A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance.

The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied.

When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

In one embodiment at least one battery may be a part of or replace the energy-transforming device 1002 to supply energy to the apparatus 10 over a power supply line. In one embodiment the battery is not rechargeable. In an alternative embodiment the battery is rechargeable. The battery supply may of course be placed both remote to and incorporated in the device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

FIGS. 686-689 show in more detail block diagrams of four different ways of hydraulically or pneumatically powering an implanted apparatus according to the invention.

FIG. 686 shows a system as described above with. The system comprises an implanted apparatus and further a separate regulation reservoir 1013, a one way pump 1009 and an alternate valve 1014.

FIG. 687 shows the apparatus 10 and the regulation reservoir 1013. By moving the wall of the regulation reservoir or changing the size of the same in any other different way, the adjustment of the apparatus may be performed without any valve, just free passage of fluid any time by moving the reservoir wall.

FIG. 688 shows the apparatus 10, a two way pump 1009 and the regulation reservoir 1013.

FIG. 689 shows a block diagram of a reversed servo system with a first closed system controlling a second closed system. The servo system comprises a regulation reservoir 1013 and a servo reservoir 1050. The servo reservoir 1050 mechanically controls an implanted apparatus 10 via a mechanical interconnection 1054. The apparatus has an expandable/contactable cavity. This cavity is preferably expanded or contracted by supplying hydraulic fluid from the larger adjustable reservoir 1052 in fluid connection with the apparatus 10. Alternatively, the cavity contains compressible gas, which can be compressed and expanded under the control of the servo reservoir 1050.

The servo reservoir 1050 can also be part of the apparatus itself.

In one embodiment, the regulation reservoir is placed subcutaneous under the patient's skin and is operated by pushing the outer surface thereof by means of a finger. This system is illustrated in FIGS. 58a-c. In FIG. 690a, a flexible subcutaneous regulation reservoir 1013 is shown connected to a bulge shaped servo reservoir 1050 by means of a conduit 1011. This bellow shaped servo reservoir 1050 is comprised in a flexible apparatus 10. In the state shown in FIG. 690a, the servo reservoir 1050 contains a minimum of fluid and most fluid is found in the regulation reservoir 1013. Due to the mechanical interconnection between the servo reservoir 1050 and the apparatus 10, the outer shape of the apparatus is contracted, i.e., it occupies less than its maximum volume. This maximum volume is shown with dashed lines in the figure.

FIG. 690b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 10 so that it occupies its maximum volume, thereby stretching the stomach wall (not shown), which it contacts.

The regulation reservoir 1013 is preferably provided with means 1013a for keeping its shape after compression. This means, which is schematically shown in the figure, will thus keep the apparatus in a stretched position also when the user releases the regulation reservoir. In this way, the regulation reservoir essentially operates as an on/off switch for the system.

An alternative embodiment of hydraulic or pneumatic operation will now be described with reference to FIGS. 691 and 692a-c. The block diagram shown in FIG. 691 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 10 having an expandable/contactable cavity is in turn controlled by the larger adjustable reservoir 1052 by supply of hydraulic fluid from the larger adjustable reservoir 1052 in fluid connection with the apparatus 10.

An example of this embodiment will now be described with reference to FIG. 692a-c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 692a, the servo reservoir 1050 contains a minimum of fluid and most fluid is found in the regulation reservoir 1013.

The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 10. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 10. In other words, in this reversed servo, a small volume in the regulation reservoir is compressed with a higher force and this creates a movement of a larger total area with less force per area unit.

Like in the previous embodiment described above with reference to FIGS. 690a-c, the regulation reservoir 1013 is preferably provided with means 1013a for keeping its shape after compression. This means, which is schematically shown in the figure, will thus keep the apparatus 10 in a stretched position also when the user releases the regulation reservoir. In this way, the regulation reservoir essentially operates as an on/off switch for the system.

Although the different parts described above have specific placements on the drawings it should be understood that these placements might vary, depending on the application.

The lubricating fluid used in any of the embodiments herein is preferably a biocompatible lubricating fluid imitating the synovial fluid of the natural hip joint. According to one embodiment the lubricating fluid is Hyaluronic acid.

In all of the embodiments above it is conceivable that the conduit is excluded and that the channel or channels are in direct connection with the reservoir or the injection port. Please note that any embodiment or part of embodiment as well as any method or part of method could be combined in any way. All examples herein should be seen as part of the general description and therefore possible to combine in any way in general terms. Please note that the description in general should be seen as describing both of an apparatus and a method.

The various aforementioned features may be combined in any way if such combination is not clearly contradictory. The invention will now be described in more detail in respect of preferred embodiments and in reference to the accompanying drawings. Again, individual features of the various embodiments may be combined or exchanged unless such combination or exchange is clearly contradictory to the overall function of the device.

### F: Control and Adjustments of a Bone Adjustment in a Mammal

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control and adjustments of a bone adjustment in a mammal. Examples of such devices for bone adjustments will now be described.

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−%, and most preferably +/−10% of the numeric values, where applicable.

The term “animal” encompasses all mammals and in particular humans. Similarly, the terms “treatment”, “therapy”, and “therapeutic use” encompass both human and animal or veterinary applications.

The term “extension device” comprises any device which is capable of longitudinal movement and in particular capable of exerting a force longitudinally between two or more points. An extension device can be a hydraulic device, an electronic device, a mechanical device, or a combination of two or more of the previous.

The term “hydraulic device” comprises any device wherein the energy that brings about the longitudinal force is transmitted by a hydraulic fluid acting on elements in the device. Examples of such elements include, but are not limited to hydraulic cylinders, hydraulically inflatable tubes, balloons, bellows and the like.

The term “implanted” indicates that a medical device 100 or an element of a medical device is introduced permanently or temporarily into a human or animal body. An implanted device can be contained within the human or animal body in its entirety, or only partially, for example by being accessible through a port or other interface in the skin of said human or animal. An implanted device can be enclosed in a human or animal body in its entirety, and communicate wirelessly with an external apparatus for transmitting and receiving signals, for example transmitting measurement data and receiving control signals, and for receiving energy.

The inventions also concern an implantable medical device 100 for bone adjustment in a mammal, which according to an embodiment comprises two or more anchoring devices for attaching to a bone in said mammal, and an adjustment device for exerting force on said anchoring devices to adjust the distance between or orientation of at least two of said anchoring devices, wherein said anchoring devices and said adjustment device are implanted in said mammal and wherein said adjustment device is constructed to postoperatively adjust said distance. This is illustrated schematically in FIG. 266 where a fractured tibia (10) having a fracture zone (20) is shown, supported by two devices (40, 50) according to the invention, both devices being attached to anchoring devices (31, 32, 33, 34) attached to the bone.

FIG. 267 shows a detailed view where an implantable medical device 100 in the form of an extension device, here schematically shown as a hydraulic device (80) having two actuators (91, 92) attached to two anchoring devices (101, 102) which can be conventional pins or screws, suitable for inserting in bone. The hydraulic device is in fluid contact through a tube (110) to a hydraulic power unit (120) supplying pressurized hydraulic fluid, which in turn communicates with a control unit (130). Optionally, said control element also supplies the hydraulic power unit with energy. The hydraulic power unit may comprise a reservoir and a pump or a hydrophore type of pre-pressurized expansion reservoir or any other hydraulic solution. The control unit, energy source, reservoir, pump or motor may all be implanted separate or together in any combination.

The power unit 120 can further be connected to or comprise a hydraulic pump P associated with a reservoir 122 containing of a fluid used to regulate the pressure of the device 80. The pump is thus adapted to pump the hydraulic fluid in or out from the device 80 in order to adjust the pressure in the device and the position of the actuators 91, 92.

The power unit 120 can also comprise a rechargeable battery 123 chargeable from the outside by an external power supply/charger unit 112 sending wireless energy.

The adjustment can be controlled by an electronic external control unit 200 adapted to receive and transmit signals from a transmitter/receiver 106 located outside the body of a treated patient.

The hydraulic device preferably comprises a device positioning system such as a fluid volume or flow measurement or any other sensor input to see the position of the adjustment device. A sensor sensing elongation, for example a capacitance sensor or impedance sensor or any sensor sensing movement or a specific position is preferably provided, a sensor communicating with the control unit 200.

Alternatively the schematic FIG. 267 may also instead show a mechanical device 80. In such a case a mechanical wire is outlined as 110 adapted to operate said mechanical device. The power unit 120 may in such a case instead comprise a motor M a servo 123 and as before the control unit 200 and sensor. The rechargeable power supply may instead be indicated by the unit 122. The motor may of course be placed directly in the mechanical unit 80, wherein the mechanical wire 110 instead is an electrical wire.

The inventions also concern a medical device 100 for bone adjustment in a mammal, which according to an embodiment comprises two or more anchoring devices for attaching to a bone in said mammal, and an adjustment device for exerting force on said anchoring devices to adjust the distance between or orientation of at least two of said anchoring devices, wherein said anchoring devices and said adjustment device are implanted intramedullary in said mammal and wherein said adjustment device is constructed to postoperatively adjust said distance.

FIG. 268 illustrates an embodiment of the invention, where a medical device 100 is implanted in a bone (200), said bone having two end portions or epiphysis (201, 202) and a fracture zone (206), said fracture zone also constituting the growth or elongation zone. The medullar cavity (204) is schematically shown in a partial cut-out view and in said cavity, a medical device (100) is provided, said device having actuators or anchoring devices (212, 214) acting on the end portions of the medullar cavity, thus elongating the bone through osteogenesis in the fracture or elongation zone (206).

Detailed views of the medical device 100, according to different embodiments are shown in FIG. 269.

FIG. 269a schematically shows an embodiment of an implantable medical device 100 in the form of an extension element or device (100), comprising a housing (301) with an electrical motor (302) acting on a threaded cylinder (303) engaged to two actuators (304, 305). Any mechanical solution may be applied. Preferably the motor uses a servo mechanism to economize force to distance. The control unit, energy source, motor or servo mechanism may all be implanted separately or together in any combination.

FIG. 269b schematically shows another embodiment of an implantable medical device 100 in the form of an extension element or device (100) comprising a housing (401) with two pistons (402, 403) connected to two actuators (404, 405). The pistons together with the housing and possible additional elements form a hydraulic device, connected via a tubing (406) to a hydraulic power unit (not shown).

In a medical device 100 according to said embodiment, the adjustment is the lengthening of a bone, the healing of a fracture, the changing of a bone angle, the reshaping of a bone, the compression of a bone, the torsion of a bone or a combination thereof.

A medical device 100 according to the invention can also be applied to the adjustment of the curvature of the spine. FIG. 270 illustrates an embodiment where a medical device 100 according to the invention is applied to the adjustment of the curvature of the spine. Detail (a) is a posterior view of the vertebrae of the lower back, vertebrae lumbales, schematically showing two devices (501, 504) according to the invention attached to opposite sides of the spine. For illustration purposes, one device (501) is shown attached to two adjoining vertebrae by two anchoring devices (502, 503) anchored in the corpus vertebrae, whereas another device (504) is shown attached to two non-adjoining vertebrae by two anchoring devices (505, 506). Detail (b) is a lateral detail schematically showing two devices (510, 520) according to the invention attached to opposite sides of the spine by anchoring devices (511, 512, 521, 522). For illustration purposes, one device acts on adjoining vertebrae, whereas the other device acts on non-adjoining vertebrae. This embodiment can be used to adjust the curvature of the spine, to relieve a herniated lumbar disc or the like.

In a medical device 100 according to any of the embodiments presented herein, said two or more anchoring devices are adapted to engage the bone from the inside of the intramedullar cavity. Preferably said at least two anchoring devices are chosen from a screw, an adhesive, a barb construction, a saw-tooth construction, an expandable element, combinations thereof or other mechanical connecting members. Non-limiting examples of such anchoring devices are illustrated schematically in FIG. 275 a-e, illustrating in turn an arrangement of flanges, a locking device, a screw, a porous end-portion for osseointegration, and a sintered or rugged end-portion with increased friction and/or improved osseointegration properties.

In a medical device 100 according to any of the embodiments presented herein, the force exerted by the adjustment device is a longitudinal force, extending the length of the bone.

According to an embodiment, said longitudinal force is directed to the end portions of the medullar cavity.

According to another embodiment, the force exerted by the adjustment device is a longitudinal force, adjusting the angle or curvature of the bone. This is illustrated in FIG. 271a schematically showing a frontal view of the right femur (600) exhibiting a curvature deviating from the natural form of this bone. The curvature may be due to a congenital disease or other condition. The dashed lines (601, 602) indicate how the bone can be fractured, preferably by sawing. In one example, wedge shaped parts are removed and the bone divided into sections, here illustrated as three sections. FIG. 271b shows how these three sections of the femur (603) are repositioned to a desired orientation, i.e. a straighter bone. The fracture zones (604, 605) are then used as growth zones in order to compensate for the loss of length due to the removal of bone. Devices (606, 607) according to the invention are then attached via actuators and anchoring devices to said sections, ensuring their position and exerting force to achieve an elongation by distractive osteogenesis. The arrows illustrate schematically that the parts of the bone can be adjusted in relation to each other, for example by adjusting the angle or orientation of said parts.

A related embodiment is illustrated in FIGS. 271c and 271d, where a deformed bone 600 is cut at two locations, 601 and 602, each cut preferably being wedge shaped in order to allow for the straightening of the bone, and devices 610 and 620 according to the inventions inserted into the medullar cavity. Similarly as in FIG. 271b, the arrows illustrate schematically that the parts of the bone can be adjusted in relation to each other, for example by adjusting the angle or orientation of said parts.

According to yet another embodiment, the force exerted by the device applies torque to the bone, adjusting the torsion of the bone along its longitudinal axis. This embodiment is illustrated in FIGS. 271e and 271f, where a bone 600 is cut along the dashed line 630 and optionally along one or more lines, exemplified as 631. One or more implantable medical device or devices 640 and 650 according to the invention are inserted into the medullar cavity. The arrows indicate that one or several parts of the bone can be adjusted, for example rotated in relation to a joint, or to a section of the bone.

According to another embodiment, freely combinable with any of the embodiments herein, said device is flexible. The advantages of a flexible device are illustrated in FIG. 274a-274d which schematically show a bone 200, having a fracture zone I. An opening II is prepared by a surgeon, allowing the insertion of a device III into the intramedullar cavity IV. FIG. 274b illustrates how the device III is flexible according to an embodiment of the invention, and how this makes it possible to introduce said device into the intramedullar cavity through an opening which is not in straight longitudinal extension to the cavity. Further, FIG. 274c illustrates how the device III, when in place in the cavity IV, retains its original shape and in addition, expands longitudinally to exert a force against the end portions of the cavity. Further, the anchoring devices securely engage the bone. The opening II is preferably closed, e.g. using bone cement. Finally, FIG. 274d illustrates an embodiment where the device III is connected to a power unit V, which can have the components and functions as the power unit 120 shown in FIG. 267.

According to another embodiment, the adjustment device comprises a hydraulic device for said bone adjustment, to control the amount of force exerted by the device onto said anchoring devices.

Preferably said hydraulic device comprises a cylinder and piston.

According to another embodiment, the hydraulic device comprises a mechanical multi step locking mechanism, locking the hydraulic device in its new position after adjustment. Said mechanical multi step locking mechanism may comprise at least one of a sprint, an elongated structure using the principle of saw teeth, flanges, barbs or a bonnet band, a nut, a gearbox, or a spring loaded locking principle.

According to another embodiment, the hydraulic device comprises hydraulic fluid and a reservoir containing said fluid, adapted to move said fluid to said adjustment device. Preferably said hydraulic fluid is moved from said reservoir to said adjustment device by using a pre-pressurized reservoir or a pump.

According to another embodiment, the hydraulic device comprises a device positioning system such as a fluid volume or flow measurement or any other sensor input to see the position of the adjustment device.

According to another embodiment, the device comprises a control device. Preferably said control device follows a program of incremental changes, set before the device is implanted. Alternatively, said control device follows a program of incremental changes, communicated to the control device after implantation and/or during the treatment. X-ray or ultrasonic follow up may indicate further treatment, decided by the responsible orthopaedic surgeon.

According to another embodiment, said control device comprises an external control unit and an implantable receiver suitable for wireless communication with said external control unit, having a transmitter located outside the body.

According to another embodiment, said control device controls incremental changes of the adjustment device, communicated to the receiver after implantation and/or during the treatment by using said external control unit.

According to another embodiment, freely combinable with any one of the embodiments presented herein, said hydraulic adjustment device is adapted to being stabilized when the bone adjustment is completed. In this embodiment, the hydraulic adjustment device can be filled with a material which stabilizes the position of the adjustment device and permanents the distance between the anchoring devices.

Preferably said material is chosen from a curable foam, a curable gel, a polymer or polymer mixture which solidifies, crosslinks or otherwise attains and retains a stable volume. Alternatively, the hydraulic fluid used in said device is a material chosen from a curable foam, a curable gel, a polymer or polymer mixture which solidifies, crosslinks or otherwise attains and retains a stable volume when the curing, solidification, crosslinking or other reaction is initiated by the user. According to another embodiment, said material chosen from curable foam, a curable gel, a polymer or polymer mixture which solidifies, crosslinks or otherwise attains and retains a stable volume, is added to the device, partially or completely replacing the hydraulic fluid.

According to another embodiment, the adjustment device comprises a mechanical device for said bone adjustment. Preferably said adjustment device is operated by an operation device, such as motor. According to another embodiment, the adjustment device comprises a control device, wherein the operation device is controlled by said control device.

According to another embodiment, the motor comprises a motor or device positioning system such as a tachometer or any other sensor input to see the position of the adjustment device.

According to another embodiment, the mechanical device for said bone adjustment comprises at least one nut and screw.

According to another embodiment, the mechanical device for said bone adjustment comprises at least one gearbox.

According to yet another embodiment, the mechanical device for said bone adjustment comprises a servo mechanism or mechanical amplifier.

According to any one of the embodiments herein, the device is adapted for exerting an intermittent and/or oscillating force.

According to any one of the embodiments herein, the device comprises a locking device which allows extension of the device but substantially prevents contraction.

Another embodiment of the inventions is a method for bone adjustment in a mammal, wherein a hydraulic or mechanical device according to any one of the above embodiments is used and implanted in the body of said mammal.

According to another embodiment, the device is implanted intramedullary in the body of said mammal, exerting a force to anchoring devices anchored to the inside of said bone.

According to another embodiment of the method, said bone adjustment is the lengthening of a bone, the healing of a fracture, the changing of a bone angle, the reshaping of a bone, the torsion of a bone or a combination thereof.

According to one embodiment, said adjustment is a step in a treatment to correct a limb discrepancy caused by a congenital condition, deformation or previous trauma.

According to another embodiment, said adjustment is reshaping or lengthening of a bone involving distraction osteogenesis treatment.

According to yet another embodiment, said adjustment is the reshaping or lengthening of a bone as a step of correcting a congenital deformation.

According to a further embodiment, said adjustment is the reshaping or lengthening of a bone as a step of a cosmetic treatment.

According to any of the above embodiments of the method, reshaping is one of changing the angle or curvature of a bone, changing the torsion of a bone, changing the angle between the diaphysis and the epiphysis, changing the thickness of a bone or a combination thereof.

Another embodiment of the inventions is a method for bone adjustment in a mammal wherein a device is implanted intramedullary in the body of said mammal, wherein said device is a hydraulic device exerting a force to anchoring devices anchored in said bone and a control device which controls the amount of force exerted by the device.

Another embodiment of the inventions is a method for bone adjustment in a mammal wherein a device is implanted intramedullary in the body of said mammal, wherein said device is a mechanical device exerting a force to anchoring devices anchored in said bone and a control device which controls the amount of force exerted by the device.

In any of the above methods, said control device follows a program of incremental changes, set before the device is implanted. Alternatively, said control device follows a program of incremental changes, communicated to the control device after implantation and/or during the treatment.

According to any of the embodiments of the method, said device is stabilized when the treatment is completed. Preferably said device is stabilized by filling the device with a material which stabilizes the position of the adjustment device and permanents the distance between the anchoring devices. In this method said material is chosen from curable foam, a curable gel, a polymer or polymer mixture which solidifies, crosslinks or otherwise attains and retains a stable volume.

Alternatively, the device is a hydraulic device and the hydraulic fluid is a material chosen from a curable foam, a curable gel, a polymer or polymer mixture which solidifies, crosslinks or otherwise attains and retains a stable volume when the curing, solidification, crosslinking or other reaction is initiated by the user.

According to another embodiment, the device is a hydraulic device and a material chosen from curable foam, a curable gel, a polymer or polymer mixture which solidifies, crosslinks or otherwise attains and retains a stable volume, is added to said device, partially or completely replacing the hydraulic fluid.

Another embodiment of the inventions is a method for distractive osteogenesis where the fractured bone is subjected to an intermittent and/or oscillating force using an implanted hydraulic or mechanical device.

Another embodiment of the inventions is a method for treating a bone dysfunction of a mammal patient by providing a device for bone adjustment comprising at least two anchoring devices according to any one of embodiments of a device according to the inventions, the method comprising the steps of
i. inserting a needle or tube-like instrument into a cavity of said mammal patient;
ii. inflating said cavity by introducing a fluid through said needle or tube-like instrument and thereby expanding said cavity;
iii. placing at least two laparoscopic trocars in said cavity;
iv. inserting a camera through one of said laparoscopic trocars into said cavity;
v. inserting at least one dissecting tool through one of said at least two laparoscopic trocars;
vi. dissecting an area of the dysfunctional bone;
vii. placing the device for bone adjustment and anchoring devices in the medullar cavity of said bone;
viii. anchoring said anchoring devices in contact with said bone;
ix. closing the mammal body preferably in layers; and
x. non-invasively adjusting said bone postoperatively.

Another embodiment of the inventions is a method of treating a bone dysfunction of a mammal patient by providing a device for bone adjustment comprising at least two anchoring devices according to any one the embodiments of the device according to the inventions, comprising the steps of:
i. cutting the skin of said human patient;
ii. dissecting an area of the dysfunctional bone;
iii. placing the device in the medullar cavity of said bone;
iv. anchoring said anchoring devices in contact with said bone;
v. closing the mammal body preferably in layers; and
vi. non-invasively adjusting said bone postoperatively.

According to a further embodiment, the method of treating a mammal patient further comprises the step of withdrawing the instruments.

According to a further embodiment, the method of treating a mammal patient further comprises the step of closing the skin using sutures or staples.

According to a further embodiment, the step of dissecting includes dissecting an area of the arm or leg comprising, dissecting an area of at least one of the following bones; clavicula, scapula, humerus, radius, ulna, pelvic bone, femur, tibia, fibula or calcaneus.

According to a further embodiment, the step of dissecting includes dissecting an area of the arm or leg comprising, dissecting an area at least one of the following joints; shoulder, elbow, hip, knee, hand and foot.

According to a further embodiment of the method, an opening into the medullar cavity is made by drilling.

Another embodiment of the inventions relates to a system comprising an apparatus or device according to any one of the embodiments presented herein.

According to a further embodiment, said system further comprises at least one switch implantable in the patient for manually and non-invasively controlling the apparatus.

According to a further embodiment, said system further comprises a hydraulic device having an implantable hydraulic reservoir, which is hydraulically connected to the apparatus, wherein the apparatus is adapted to be non-invasively regulated by manually pressing the hydraulic reservoir.

According to a further embodiment, said system further comprises a wireless external control unit for non-invasively controlling the apparatus.

According to an embodiment, said wireless external control unit comprises at least one external signal transmitter and/or receiver, further comprising an internal signal receiver and/or transmitter implantable in the patient for receiving signals transmitted by the external signal transmitter or transmitting signals to the external signal receiver.

According to an embodiment, said wireless external control unit transmits at least one wireless control signal for controlling the apparatus.

According to an embodiment, said wireless control signal comprises a frequency, amplitude, or phase modulated signal or a combination thereof.

According to an embodiment, said wireless external control unit transmits an electromagnetic carrier wave signal for carrying the control signal.

According to yet another embodiment, said system further comprises a wireless energy-transmission device for non-invasively energizing implantable energy consuming components of the apparatus with wireless energy.

Wireless transmission of energy gives additional advantages to the inventive method and device.

These embodiments will be disclosed in the following with reference to the schematic illustrations attached.

FIG. 273 illustrates a system with an implantable medical device 100 for treating a disease comprising an apparatus 1001 according to an embodiment of the present invention placed in the lower leg of a patient. An implanted energy-transforming device 1002 is adapted to supply energy consuming components of the apparatus with energy via a power supply line 1003. An external energy-transmission device 1004 for non-invasively energizing the apparatus 1001 transmits energy by at least one wireless energy signal. The implanted energy-transforming device 1002 transforms energy from the wireless energy signal into electric energy which is supplied via the power supply line 1003. Another external device 1004a is illustrated, schematically showing a device capable of transmitting control signals to the apparatus 1001, and optionally receiving signals transmitted by the apparatus 1001, for example information about the position, energy level, tension, pressure, temperature or other relevant information registered by one or more sensors (not shown) included in the apparatus.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field.

The wireless energy-transmission device 1004 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal.

Generally speaking, the energy-transforming device 1002 is provided for transforming wireless energy of a first form transmitted by the energy-transmission device 1004 into energy of a second form, which typically is different from the energy of the first form. The implanted apparatus 1001 is operable in response to the energy of the second form. The energy-transforming device 1002 may directly power the apparatus with the second form energy, as the energy-transforming device 1002 transforms the first form energy transmitted by the energy-transmission device 1004 into the second form energy. The system may further include an implantable accumulator, wherein the second form energy is used at least partly to charge the accumulator.

Alternatively, the wireless energy transmitted by the energy-transmission device 1004 may be used to directly power the apparatus, as the wireless energy is being transmitted by the energy-transmission device 1004. Where the system comprises an operation device for operating the apparatus, as will be described below, the wireless energy transmitted by the energy-transmission device 1004 may be used to directly power the operation device to create kinetic energy for the operation of the apparatus.

The wireless energy of the first form may comprise sound waves and the energy-transforming device 1002 may include a piezo-electric element for transforming the sound waves into electric energy.

The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus.

Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus.

The external energy-transmission device 1004 also includes a wireless external control unit having an external signal transmitter for transmitting a wireless control signal for non-invasively controlling the apparatus. The control signal is received by an implanted signal receiver which may be incorporated in the implanted energy-transforming device 1002 or be separate there from.

The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field.

The wireless external control unit may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless external control unit preferably transmits an electromagnetic carrier wave signal for carrying the digital or analogue control signals.

FIG. 667 illustrates the system of FIG. 263 in the form of a more generalized block diagram showing the apparatus 301, the energy-transforming device 1002 powering the apparatus 301 via power supply line 1003, and the external energy-transmission device 200, The patient's skin SK, generally shown by a vertical line, separates the interior of the patient to the right of the line from the exterior to the left of the line.

FIG. 668 shows an embodiment identical to that of FIG. 272, except that a reversing device in the form of an electric switch 306 operable for example by polarized energy also is implanted in the patient for reversing the apparatus 301. When the switch is operated by polarized energy the wireless external control unit of the external energy-transmission device 200 transmits a wireless signal that carries polarized energy and the implanted energy-transforming device 1002 transforms the wireless polarized energy into a polarized current for operating the electric switch 306. When the polarity of the current is shifted by the implanted energy-transforming device 1002 the electric switch 306 reverses the function performed by the apparatus 301.

FIG. 669 shows an embodiment identical to that of FIG. 667, except that an operation device 1007 implanted in the patient for operating the apparatus 301 is provided between the implanted energy-transforming device 1002 and the apparatus 301. This operation device can be in the form of a motor 1007, such as an electric servomotor. The motor 1007 is powered with energy from the implanted energy-transforming device 1002, as the external control unit of the external energy-transmission device 200 transmits a wireless signal to the receiver of the implanted energy-transforming device 1002.

FIG. 670 shows an embodiment identical to that of FIG. 667, except that it also comprises an operation device is in the form of an assembly 1008 including a motor/pump unit 1009 and a fluid reservoir 1010 is implanted in the patient. In this case the apparatus 301 is hydraulically operated, i.e. hydraulic fluid is pumped by the motor/pump unit 1009 from the fluid reservoir 1010 through a conduit 1011 to the apparatus 301 to operate the apparatus, and hydraulic fluid is pumped by the motor/pump unit 1009 back from the apparatus 301 to the fluid reservoir 1010 to return the apparatus to a starting position. The implanted energy-transforming device 1002 transforms wireless energy into a current, for example a polarized current, for powering the motor/pump unit 1009 via an electric power supply line 1012.

Instead of a hydraulically operated apparatus 301, it is also envisaged that the operation device comprises a pneumatic operation device. In this case, the hydraulic fluid can be pressurized air to be used for regulation and the fluid reservoir is replaced by an air chamber.

In all of these embodiments the energy-transforming device 1002 may include a rechargeable accumulator like a battery or a capacitor to be charged by the wireless energy and supplies energy for any energy consuming part of the system.

As an alternative, the wireless external control unit described above may be replaced by manual control of any implanted part to make contact with by the patient's hand most likely indirect, for example a press button placed under the skin.

FIG. 671 shows an embodiment comprising the external energy-transmission device 200 with its wireless external control unit, the apparatus 301, in this case hydraulically operated, and the implanted energy-transforming device 1002, and further comprising a hydraulic fluid reservoir 1013, a motor/pump unit 1009 and an reversing device in the form of a hydraulic valve shifting device 1014, all implanted in the patient. Of course the hydraulic operation could easily be performed by just changing the pumping direction and the hydraulic valve may therefore be omitted. The external control unit may be a device separated from the external energy-transmission device or included in the same. The motor of the motor/pump unit 1009 is an electric motor. In response to a control signal from the wireless external control unit of the external energy-transmission device 200, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 301. The external control unit of the external energy-transmission device 200 controls the hydraulic valve shifting device 1014 to shift the hydraulic fluid flow direction between one direction in which the fluid is pumped by the motor/pump unit 1009 from the hydraulic fluid reservoir 1013 to the apparatus 301 to operate the apparatus, and another opposite direction in which the fluid is pumped by the motor/pump unit 1009 back from the apparatus 301 to the hydraulic fluid reservoir 1013 to return the apparatus to a starting position.

FIG. 672 shows an embodiment comprising the external energy-transmission device 200 with its wireless external control unit, the apparatus 301, the implanted energy-transforming device 1002, an implanted internal control unit 102 controlled by the wireless external control unit of the external energy-transmission device 200, an implanted accumulator 1016 and an implanted capacitor 1017. The internal control unit 102 arranges storage of electric energy received from the implanted energy-transforming device 1002 in the accumulator 1016, which supplies energy to the apparatus 301. In response to a control signal from the wireless external control unit of the external energy-transmission device 200, the internal control unit 102 either releases electric energy from the accumulator 1016 and transfers the released energy via power lines 1018 and 1019, or directly transfers electric energy from the implanted energy-transforming device 1002 via a power line 1020, the capacitor 1017, which stabilizes the electric current, a power line 1021 and the power line 319, for the operation of the apparatus 301.

The internal control unit is preferably programmable from outside the patient's body. In a preferred embodiment, the internal control unit is programmed to regulate the apparatus 301 according to a pre-programmed time-schedule or to input from any sensor sensing any possible physical parameter of the patient or any functional parameter of the system.

In accordance with an alternative, the capacitor 1017 in the embodiment of FIG. 672 may be omitted. In accordance with another alternative, the accumulator 1016 in this embodiment may be omitted.

FIG. 673 shows an embodiment identical to that of FIG. 667, except that a battery 1022 for supplying energy for the operation of the apparatus 301 and an electric switch 1023 for switching the operation of the apparatus 301 also are implanted in the patient. The electric switch 1023 may be controlled by the external control unit and may also be operated by the energy supplied by the implanted energy-transforming device 1002 to switch from an off mode, in which the battery 1022 is not in use, to an on mode, in which the battery 1022 supplies energy for the operation of the apparatus 301.

FIG. 674 shows an embodiment identical to that of FIG. 673, except that an internal control unit 102 controllable by the wireless external control unit of the external energy-transmission device 200 also is implanted in the patient. In this case, the electric switch 1023 is operated by the energy supplied by the implanted energy-transforming device 1002 to switch from an off mode, in which the wireless external control unit is prevented from controlling the internal control unit 102 and the battery is not in use, to a standby mode, in which the external control unit is permitted to control the internal control unit 102 to release electric energy from the battery 1022 for the operation of the apparatus 301.

FIG. 675 shows an embodiment identical to that of FIG. 674, except that an accumulator 1016 is substituted for the battery 1022 and the implanted components are interconnected differently. In this case, the accumulator 1016 stores energy from the implanted energy-transforming device 1002. In response to a control signal from the wireless external control unit of the external energy-transmission device 200, the internal control unit 102 controls the electric switch 1023 to switch from an off mode, in which the accumulator 1016 is not in use, to an on mode, in which the accumulator 1016 supplies energy for the operation of the apparatus 301. The accumulator may be combined with or replaced by a capacitor.

FIG. 676 shows an embodiment identical to that of FIG. 675, except that a battery 1022 also is implanted in the patient and the implanted components are interconnected differently. In response to a control signal from the wireless external control unit of the external energy-transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the apparatus 301.

Alternatively, the electric switch 1023 may be operated by energy supplied by the accumulator 1016 to switch from an off mode, in which the wireless external control unit is prevented from controlling the battery 1022 to supply electric energy and is not in use, to a standby mode, in which the wireless external control unit is permitted to control the battery 1022 to supply electric energy for the operation of the apparatus 301.

It should be understood that the switch 1023 and all other switches in this application should be interpreted in its broadest embodiment. This means a transistor, MCU, MCPU, ASIC, FPGA or a DA converter or any other electronic component or circuit that may switch the power on and off. Preferably the switch is controlled from outside the body, or alternatively by an implanted internal control unit.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the apparatus 301 (mechanically operated). Even simpler is to switch the direction of the motor electronically. The gear box interpreted in its broadest embodiment may stand for a servo arrangement saving force for the operation device in favor of longer stroke to act.

This is also illustrated in FIG. 282 which schematically shows a device according to an embodiment where an implantable medical device 100 comprises a motor 2010 operationally connected to a gear box 2020 and an adjustment device 2030, where the speed and/or effect of the motor 2010 is controlled by a control unit 2040. According to one embodiment, said control unit 2040 both senses the speed of the motor 2010 and adjusts the same, and optionally also senses the output speed of the gear box 2020, driving the adjustment device 2030. According to another embodiment, said control unit 2040 comprises a feed-back loop, sensing the speed of the motor, and adjusting the same to a desired value. In another embodiment, no gear box is present, and the control unit 2040 both senses the speed of the motor and adjusts the same.

FIG. 678 shows an embodiment identical to that of FIG. 676 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the apparatus 301.

FIG. 679 schematically shows conceivable combinations of implanted components of the apparatus for achieving various communication options. Basically, there are the apparatus 301, the internal control unit 102, motor or pump unit 1009, and the external energy-transmission device 200 including the external wireless external control unit. As already described above the wireless external control unit transmits a control signal which is received by the internal control unit 102, which in turn controls the various implanted components of the apparatus.

A feedback device, preferably comprising a sensor or measuring device 325, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, pressure, volume, diameter, stretch, elongation, extension, movement, bending, elasticity, temperature and flow.

The feedback may be sent to the internal control unit or out to an external control unit preferably via the internal control unit. Feedback may be sent out from the body via the energy transfer system or a separate communication system with receiver and transmitters.

The internal control unit 102, or alternatively the external wireless external control unit of the external energy-transmission device 200, may control the apparatus 301 in response to signals from the sensor 1025. A transceiver may be combined with the sensor 1025 for sending information on the sensed physical parameter to the external wireless external control unit. The wireless external control unit may comprise a signal transmitter or transceiver and the internal control unit 102 may comprise a signal receiver or transceiver. Alternatively, the wireless external control unit may comprise a signal receiver or transceiver and the internal control unit 102 may comprise a signal transmitter or transceiver. The above transceivers, transmitters and receivers may be used for sending information or data related to the apparatus 301 from inside the patient's body to the outside thereof.

Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly.

FIG. 680 shows an alternative embodiment wherein the apparatus 301 is regulated from outside the patient's body. The system comprises a battery 1022 connected to the apparatus 301 via a subcutaneous electric switch 1026. Thus, the regulation of the apparatus 301 is performed non-invasively by manually pressing the subcutaneous switch, whereby the operation of the apparatus 301 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 681 shows an alternative embodiment, wherein the system comprises a hydraulic fluid reservoir 1013 hydraulically connected to the apparatus. Non-invasive regulation is performed by manually pressing the hydraulic reservoir connected to the apparatus.

The system may include an external data communicator and an implantable internal data communicator communicating with the external data communicator. The internal communicator feeds data related to the apparatus or the patient to the external data communicator and/or the external data communicator feeds data to the internal data communicator.

FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 302 connected to implanted energy consuming components of the apparatus 301. Such an energy receiver 302 may include an energy source and/or an energy-transforming device. Briefly described, wireless energy is transmitted from an external energy source 200a located outside the patient and is received by the internal energy receiver 302 located inside the patient. The internal energy receiver is adapted to directly or indirectly supply received energy to the energy consuming components of the apparatus 301. An energy balance is determined between the energy received by the internal energy receiver 302 and the energy used for the apparatus 301, and the transmission of wireless energy is then controlled based on the determined energy balance. The energy balance thus provides an accurate indication of the correct amount of energy needed, which is sufficient to operate the apparatus 301 properly, but without causing undue temperature rise.

In FIG. 682 the patient's skin is indicated by a vertical line SK. Here, the energy receiver comprises an energy-transforming device 1002 located inside the patient, preferably just beneath the patient's skin SK. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy-source 200a provided in an external energy-transmission device 200 located outside the patient's skin SK in the vicinity of the implanted energy-transforming device 1002.

As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used.

The amount of energy received by the implanted energy receiver may be compared with the energy used by the implanted components of the apparatus. The term “energy used” is then understood to include also energy stored by implanted components of the apparatus. A control device includes an external control unit 200b that controls the external energy source 200a based on the determined energy balance to regulate the amount of transferred energy. In order to transfer the correct amount of energy, the energy balance and the required amount of energy is determined by means of a determination device including an implanted internal control unit 102 connected to the apparatus 301. The internal control unit 102 may thus be arranged to receive various measurements obtained by suitable sensors or the like, not shown, measuring certain characteristics of the apparatus 301, somehow reflecting the required amount of energy needed for proper operation of the apparatus 301. Moreover, the current condition of the patient may also be detected by means of suitable measuring devices or sensors, in order to provide parameters reflecting the patient's condition. Hence, such characteristics and/or parameters may be related to the current state of the apparatus 301, such as power consumption, operational mode and temperature, as well as the patient's condition reflected by parameters such as; body temperature, blood pressure, heartbeats and breathing. Other kinds of physical parameters of the patient and functional parameters of the device are described elsewhere.

Furthermore, an energy source in the form of an accumulator 1016 may optionally be connected to the implanted energy-transforming device 1002 for accumulating received energy for later use by the apparatus 301. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 301, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 of the determination device is adapted to determine the energy balance and/or the currently required amount of energy, (either energy per time unit or accumulated energy) based on measurements made by the above-mentioned sensors or measuring devices of the apparatus 301, or the patient, or an implanted energy source if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal.

Alternatively, the determination device may include the external control unit 200b. In this alternative, sensor measurements can be transmitted directly to the external control unit 200b wherein the energy balance and/or the currently required amount of energy can be determined by the external control unit 200b, thus integrating the above-described function of the internal control unit 102 in the external control unit 200b. In that case, the internal control unit 102 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements.

Hence, the present solution according to the arrangement of FIG. 678 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus.

The internal signal transmitter 1027 and the external signal receiver 200c may be implemented as separate units using suitable signal transfer means, such as radio, IR (Infrared) or ultrasonic signals.

Alternatively, the internal signal transmitter 1027 and the external signal receiver 200c may be integrated in the implanted energy-transforming device 1002 and the external energy source 200a, respectively, so as to convey control signals in a reverse direction relative to the energy transfer, basically using the same transmission technique. The control signals may be modulated with respect to frequency, phase or amplitude.

Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off In implementing this system in the arrangement of FIG. 682, the switch 1026 is either separate and controlled by the internal control unit 102, or integrated in the internal control unit 102. It should be understood that the switch 1026 should be interpreted in its broadest embodiment. This means a transistor, MCU, MCPU, ASIC FPGA or a DA converter or any other electronic component or circuit that may switch the power on and off.

To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 1027 to the external signal receiver 200c. Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized.

This coupling factors information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil.

The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factors between the first and second coils. The transmitted energy may be regulated depending on the obtained coupling factor.

With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 1026 is interconnected between the external energy source 200a and an operation device, such as an electric motor 1007 operating the apparatus 301. An external control unit 200b controls the operation of the external switch 1026 to effect proper operation of the apparatus 301.

FIG. 684 illustrates different embodiments for how received energy can be supplied to and used by the apparatus 301. Similar to the example of FIG. 682, an internal energy receiver 302 receives wireless energy E from an external energy source 200a which is controlled by a transmission control unit 200b. The internal energy receiver 302 may comprise a constant voltage circuit, indicated as a dashed box “constant V” in the figure, for supplying energy at constant voltage to the apparatus 301. The internal energy receiver 302 may further comprise a constant current circuit, indicated as a dashed box “constant C” in the figure, for supplying energy at constant current to the apparatus 301.

The apparatus 301 comprises an energy consuming part 301a, which may be a motor, pump, restriction device, or any other medical appliance that requires energy for its electrical operation. The apparatus 301 may further comprise an energy storage device 301b for storing energy supplied from the internal energy receiver 302. Thus, the supplied energy may be directly consumed by the energy consuming part 301a, or stored by the energy storage device 301b. or the supplied energy may be partly consumed and partly stored. The apparatus 301 may further comprise an energy stabilizing unit 301c for stabilizing the energy supplied from the internal energy receiver 302. Thus, the energy may be supplied in a fluctuating manner such that it may be necessary to stabilize the energy before consumed or stored.

The energy supplied from the internal energy receiver 302 may further be accumulated and/or stabilized by a separate energy stabilizing unit 1028 located outside the apparatus 301, before being consumed and/or stored by the apparatus 301. Alternatively, the energy stabilizing unit 1028 may be integrated in the internal energy receiver 302. In either case, the energy stabilizing unit 1028 may comprise a constant voltage circuit and/or a constant current circuit.

It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope of the present invention.

FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such change takes place. If the amount of received energy is lower than the energy used by the implant, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window.

The schematic FIG. 685 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic figure; the transmitting parts of the system are excluded.

The implementation of the general concept of energy balance and the way the information is transmitted to the external energy transmitter can of course be implemented in numerous different ways.

The schematic FIG. 681 and the above described method of evaluating and transmitting the information should only be regarded as examples of how to implement the control system.

### Circuit Details

In FIG. 685 the symbols Y1, Y2, Y3 and so on symbolize test points within the circuit. The components in the diagram and their respective values are values that work in this particular implementation which of course is only one of an infinite number of possible design solutions.

Energy to power the circuit is received by the energy receiving coil L1. Energy to implanted components is transmitted in this particular case at a frequency of 25 kHz. The energy balance output signal is present at test point Y1.

Those skilled in the art will realize that the above various embodiments of the system could be combined in many different ways. For example, the electric switch 306 of FIG. 273 could be incorporated in any of the embodiments of FIGS. 667-673, the hydraulic valve shifting device 1014 could be incorporated in another embodiment of the inventions, and the gear box 1024 could be incorporated in yet another embodiment. Please observe that the switch simply could mean any electronic circuit or component.

The embodiments described in connection with FIGS. 682, 684 and 685 identify a method and a system for controlling transmission of wireless energy to implanted energy consuming components of an electrically operable apparatus. Such a method and system will be defined in general terms in the following.

A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance.

The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied.

When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

According to an embodiment, said wireless energy comprises a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal.

According to an embodiment, said wireless energy comprises one of the following: an electric field, a magnetic field, a combined electric and magnetic field.

According to an embodiment, said control signal comprises one of the following: an electric field, a magnetic field, a combined electric and magnetic field.

According to a further embodiment, said the signal comprises an analogue signal, a digital signal, or a combination of an analogue and digital signal.

According to yet another embodiment, said system further comprises an implantable internal energy source for powering implantable energy consuming components of the apparatus.

According to yet another embodiment, said system further comprises an external energy source for transferring energy in a wireless mode, wherein the internal energy source is chargeable by the energy transferred in the wireless mode.

According to yet another embodiment, said system further comprises a sensor or measuring device sensing or measuring a functional parameter correlated to the transfer of energy for charging the internal energy source, and a feedback device for sending feedback information from inside the patient's body to the outside thereof, the feedback information being related to the functional parameter sensed by the sensor or measured by the measuring device.

According to yet another embodiment, said system further comprises a feedback device for sending feedback information from inside the patient's body to the outside thereof, the feedback information being related to at least one of a physical parameter of the patient and a functional parameter related to the apparatus.

According to yet another embodiment, said system further comprises a sensor and/or a measuring device and an implantable internal control unit for controlling the apparatus in response to information being related to at least one of a physical parameter of the patient sensed by the sensor or measured by the measuring device and a functional parameter related to the apparatus sensed by the sensor or measured by the measuring device.

According to another embodiment, said physical parameter is a pressure or a motility movement.

According to yet another embodiment, said system further comprises an external data communicator and an implantable internal data communicator communicating with the external data communicator, wherein the internal communicator feeds data related to the apparatus or the patient to the external data communicator and/or the external data communicator feeds data to the internal data communicator.

According to yet another embodiment, said system further comprises a motor or a pump for operating the apparatus.

According to yet another embodiment, said system further comprises a hydraulic operation device for operating the apparatus.

According to yet another embodiment, said system further comprises an operation device for operating the apparatus, wherein the operation device comprises a servo designed to decrease the force needed for the operation device to operate the apparatus instead the operation device acting a longer way, increasing the time for a determined action.

According to yet another embodiment, said system further comprises an operation device for operating the apparatus, wherein the wireless energy is used in its wireless state to directly power the operation device to create kinetic energy for the operation of the apparatus, as the wireless energy is being transmitted by the energy-transmission device.

According to yet another embodiment, said system further comprises an energy-transforming device for transforming the wireless energy transmitted by the energy-transmission device from a first form into a second form of energy.

According to an embodiment, said energy-transforming device directly powers implantable energy consuming components of the apparatus with the second form energy, as the energy-transforming device transforms the first form energy transmitted by the energy-transmission device into the second form energy.

According to an embodiment, said second form energy comprises at least one of a direct current, pulsating direct current and an alternating current.

According to yet another embodiment, said system further comprises an implantable accumulator, wherein the second form energy is used at least partly to charge the accumulator.

According to an embodiment, said energy of the first or second form comprises at least one of magnetic energy, kinetic energy, sound energy, chemical energy, radiant energy, electromagnetic energy, photo energy, nuclear energy thermal energy, non-magnetic energy, non-kinetic energy, non-chemical energy, non-sonic energy, non-nuclear energy and non-thermal energy.

According to yet another embodiment, said system further comprises implantable electrical components including at least one voltage level guard and/or at least one constant current guard.

According to yet another embodiment, said system further comprises a control device for controlling the transmission of wireless energy from the energy-transmission device, and an implantable internal energy receiver for receiving the transmitted wireless energy, the internal energy receiver being connected to implantable energy consuming components of the apparatus for directly or indirectly supplying received energy thereto, the system further comprising a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

According to an embodiment, said determination device is adapted to detect a change in the energy balance, and the control device controls the transmission of wireless energy based on the detected energy balance change.

According to a further embodiment, the determination device is adapted to detect a difference between energy received by the internal energy receiver and energy used for the implantable energy consuming components of the apparatus, and the control device controls the transmission of wireless energy based on the detected energy difference.

According to a further embodiment, the energy-transmission device comprises a coil placed externally to the human body, further comprising an implantable energy receiver to be placed internally in the human body and an electric circuit connected to power the external coil with electrical pulses to transmit the wireless energy, the electrical pulses having leading and trailing edges, the electric circuit adapted to vary first time intervals between successive leading and trailing edges and/or second time intervals between successive trailing and leading edges of the electrical pulses to vary the power of the transmitted wireless energy, the energy receiver receiving the transmitted wireless energy having a varied power.

According to a further embodiment, the electric circuit is adapted to deliver the electrical pulses to remain unchanged except varying the first and/or second time intervals.

According to a further embodiment, the electric circuit has a time constant and is adapted to vary the first and second time intervals only in the range of the first time constant, so that when the lengths of the first and/or second time intervals are varied, the transmitted power over the coil is varied.

According to a further embodiment, the system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off.

According to a further embodiment, the system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil, wherein the external second coil of the energy transmitter transmits wireless energy which is received by the first coil of the energy receiver, the system further comprising a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factors between the first and second coils.

According to an embodiment, the transmitted energy may be regulated depending on the obtained coupling factor.

According to a further embodiment, said external second coil is adapted to be moved in relation to the internal first coil to establish the optimal placement of the second coil, in which the coupling factor is maximized.

According to a further embodiment, said external second coil is adapted to calibrate the amount of transferred energy to achieve the feedback information in the determination device, before the coupling factor is maximized.

According to yet another embodiment, the mechanical device comprises a mechanical multi step locking mechanism, locking the mechanical device in its new position after adjustment. An example is illustrated in FIG. 272 where FIG. 272a shows schematically a detail view of a medical device (100) according to the invention, said device comprising at least one hydraulic piston (701) and two actuators (702, 703). In this embodiment one actuator is attached to the housing of the device, whereas the other is moving. In order to prevent an actuator from returning to a previous position, for example when temporarily subjected to an increased stress, the housing has an aperture (705) and the actuator has cone-shaped flanges (704) allowing an outward movement of the actuator, but substantially preventing an inward movement.

FIG. 272b shows an alternative embodiment where, in a medical device (100) according to the invention, two pistons (801, 802) are provided in a housing. The pistons are connected to two actuators (805, 806), which in turn are engaged to anchoring devices (not shown). On the inside, said housing has a pattern of protrusions, velts or barbs, allowing said pistons to move in one direction, preferably outward, but substantially preventing an inward movement.

According to yet another embodiment, the mechanical multi step locking mechanism comprises at least one of a sprint, an elongated structure using the principle of saw teeth, flanges, barbs or a bonnet band, a nut, a gearbox, or a spring loaded locking principle.

According to a further embodiment of the system, the device comprises a control device.

According to another embodiment, said control device follows a program of incremental changes, set before the device is implanted.

According to another embodiment, said control device follows a program of incremental changes, communicated to the control device after implantation and/or during the treatment.

According to another embodiment, said control device comprises an external control unit and an implantable receiver suitable for wireless communication with said external control unit, having a transmitter located outside the body.

According to another embodiment, said control device controls incremental changes of the adjustment device, communicated to the receiver after implantation and/or during the treatment by using said external control unit.

According to a preferred embodiment, said device is flexible to allow introduction into the medullar cavity. Alternatively or in combination therewith, said device is at least partly elastic.

Alternatively or in combination therewith, said device comprises a spring. Alternatively or in combination therewith, said device is adapted to regain its shape after having been bent.

According to another embodiment, freely combinable with the other embodiments presented herein, the anchoring device comprises a thread for engaging and stabilizing the anchoring device in relation to the bone.

According to a further embodiment, the anchoring device comprises an expandable part expanding at least partially perpendicular to the longitudinal extension of the elongated device for engaging and stabilizing the anchoring device in relation to the bone. This embodiment is illustrated in

FIG. 276, which schematically shows a medical device 100, having two end-portions or anchoring devices 902 and 903, for example elastic and expandable means, here shown as activated externally, by a key or knob 901, having a portion insertable into the medical device 100 and engaging a mechanism activating and in particular expanding the anchoring devices 902 and 903.

The views I, II and III show in turn how the anchoring devices 902 and 903 are in their initial, non-expanded state, allowing insertion into a cavity; how they can be activated and expanded; and how they when expanded engage the surrounding cavity and allow the operation of the device for bone adjustment.

According to an alternative embodiment, schematically shown in FIG. 277, a medical device 100 comprises anchoring devices in the form of an arrangement of flanges 922 and 923. An external device, here shown as a wire 921 having an end portion capable of engaging a mechanism for activation of 922 and 923, is brought in operational contact with the device, and the anchoring devices activated. When activate, the anchoring devices engage the surrounding cavity and allow the operation of the device for bone adjustment.

FIG. 281 shows a partial cut-out view of a bone 1020 having a fracture zone 1021, with an implantable medical device 100 according to an embodiment implanted in the medullar cavity 1040, said device 100 comprising a body 1030 and two anchoring devices 1050 and 1051. The entry hole 1022 is shown as sealed or closed, for example with bone cement, and the adjustment device is shown in extended and operational condition, where the anchoring devices 1050 and 1051 securely engage the bone.

According to a further embodiment, the adjustment device is adapted to comprise torsion of a bone. Alternatively, or in combination, said adjustment device is adapted to change the angle of a bone.

According to a further embodiment, said adjustment device comprises at least two parts, wherein the parts are adapted to rotate in relation to each other. Preferably said relative rotation is anchored by said at least two anchoring devices.

According to another embodiment, freely combinable with the other embodiments presented herein, said adjustment device is adapted to change the angle of a bone.

According to further embodiment, freely combinable with the other embodiments presented herein, said adjustment device comprises at least two parts, wherein the parts are adapted to be positioned at an angle in relation to each other.

This is illustrated schematically in FIG. 6813 which shows an embodiment where the implanted adjustment device 100 comprises at least two parts 2100 and 2200, wherein the parts are adapted to be positioned at an angle in relation to each other, and/or rotated in relation to each other. The movement is achieved by the provision of a joint, here shown as a semicircular or hemispherical element 2250, attached to an operation device 2260, adapted to turn or rotate the element 2250 in relation to the part 2200. The element 2250 is also operationally engaged to the part 2100, where an adjustment device 2150 is adapted to engage said element, in order to change the angle between the parts 2100 and 2200.

Preferably said operation device 2260 and said adjustment device 2260 each comprise a motor, and optionally also a gear box and a control unit, as described in the context of other embodiments herein.

The arrows schematically indicate the possible directions of movement of the parts shown in FIG. 6813, but it is understood that the parts can be angled, tilted or rotated in relation to each other as desired.

According to a further embodiment, said two or more anchoring devices are adapted to engage and carry weight purely on the inside of the bone.

According to yet another embodiment, said two or more anchoring devices are adapted to engage with and carry weight to the bone without penetrating to the outside of the bone.

According to yet another embodiment, said two or more anchoring devices are adapted to engage and carry weight purely on the outside of the bone.

According to another embodiment, freely combinable with any embodiment presented herein, said device comprises a sensor directly or indirectly sensing the position of the adjustment device.

According to a further embodiment, the device comprises a feedback transmitter adapted to transmit information received directly or indirectly from said sensor out from the human body, said transmitted information adapted to be received by an external control unit and relating to the position of the adjustment device.

According to another embodiment of the device, said operation device is a motor operated as a three-phase motor. Alternatively, said operation device is a motor operated as a two- or more phase motor.

According to another embodiment, freely combinable with any embodiment presented herein, said device comprises a gearbox connected to the motor, a motor package, wherein the outgoing speed from the motor package is lower than the speed by said motor alone, accomplished by said gearbox.

According to another embodiment, freely combinable with any embodiment presented herein, said device comprises an electrical speed controller connected to the motor, a motor package, wherein the outgoing speed of the motor in said motor package is decreased by said electrical speed controller.

According to any of the above embodiments, the motor is a rotational motor and the outgoing speed of the motor package is decreased to less than 100 turns per second, alternatively decreased to less than 10 turns per second, alternatively to less than 1 turn per second, or alternatively to less than 0.1 turn per second, or alternatively to less than 0.01 turn per second, or alternatively to less than 0.001 turn per second.

According to another embodiment, freely combinable with any embodiment presented herein, said device comprises an electrical speed controller connected to the motor, a motor package, wherein the outgoing speed of the motor of said motor package is controlled by said electrical speed controller.

According to any of the above embodiments, the motor is a linear motor and the outgoing speed of the motor package is less than 1 mm per second, alternatively less than 0.1 mm per second, or alternatively less than 0.01 mm per second, or alternatively less than 0.001 mm per second, or alternatively less than 0.0001 mm per second, or less than 0.00001 mm per second.

The construction of a device according to the invention is illustrated also in FIG. 278, schematically showing an embodiment of the medical device 100, comprising two telescopically arranged parts 932 and 933, housing a longitudinal threaded central shaft or axis 937 and a motor or gear arrangement 938 acting thereon, transforming rotational force into longitudinal force and extension or contraction of the device.

Further, FIG. 279 shows another related embodiment where the medical device 100 comprises three main parts, a central section 935, and two telescopically arranged end sections 934 and 936, each connected to a longitudinal threaded central shaft or axis 939 and 940 through a motor or gear arrangement 941 and 942.

Finally, FIG. 280A and B show embodiments where a medical device for bone adjustment according to the present invention, schematically shown as 100, is enclosed in an elastic outer cover 1001, or an expandable outer cover 1011, the expansion made possible by folds 1012, the covers protecting the device from direct contact with tissues and body fluids.

Said elastic, flexible or expandable cover is preferably made of a polymeric material generally recognized as safe and approved for surgical use, and can be suitably coated to minimize tissue irritation.

Non-limiting examples of the material include silicon, polyurethane and TEFLON®, and non-limiting examples of suitable coatings include atomized metal coatings, and polymeric coatings, such as PARYLENE®.

It should be noted that the above embodiments, and features appearing in the individual embodiments, are freely combinable.

### EXAMPLES

There are animal models for the investigation of fracture healing, such as a rabbit fibula model, a sheep model for fracture treatment in osteoporosis, a murine femur fracture model. It is conceived that the inventive device and method can be tested in existing animal models, preferably models involving the use of larger mammals, such as sheep, pig, dog, monkey etc.

There are also non-invasive methods for the evaluation of bone fracture healing, see e.g. a review article (Protopappas et al., 2008) describing quantitative ultrasonic monitoring of bone fracture healing.

In a suitable animal model, a test animal is anesthetized, and a bone dissected and fractured. When dissecting a bone, care is taken to cause minimal tissue damage, e.g. by folding or pulling tissue to the side instead of removing it from its location. Pins or other anchoring devices are fixed in the bone on both sides of the fracture, and a device according to the invention attached to said anchoring devices. The tissue is replaced carefully, preferably layer by layer, and the body of the animal closed. After a suitable initial healing period, the fracture zone is adjusted non-invasively. Parameters such as bone healing, pain and signs of infection or inflammation are observed regularly. Following euthanasia, the bone is dissected and the fracture zone analyzed.

The experiment can be repeated, with necessary modifications, in the same or in a different animal model, for evaluating the adjustment of the curvature of the spine, realigning a joint, changing the curvature of a bone, or the like.

Although the invention has been described with regard to its preferred embodiments, which constitute the best mode presently known to the inventors, it should be understood that various changes and modifications as would be obvious to one having the ordinary skill in this art may be made without departing from the scope which is set forth in the claims appended hereto.

### G: Drain—Assisting Control Fluid Movement Devices

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control fluid movement devices, such as drainage devices or hydraulic treatment devices.

In FIGS. 284a and 284b a view illustrating an implantable medical device in the form of an implantable fluid movement device 100. The device 100 comprises a bellow 101 adapted to move between a compressed position in which the bellow has a small inside volume and an expanded position in which the bellow has a larger inside volume. The view in FIG. 284a shows the bellow in a compressed position and the view in FIG. 284b shows the bellow in an expanded position.

The device 100 further comprises a member such as screw 103 adapted to compress the bellow 101. The screw 103 is accordance with one embodiment driven by a motor 105. The motor may many type of suitable motor including but not limited an electrical motor and a hydraulic motor. In accordance with one embodiment the motor is associated with a clutch 107 for regulating the power applied to the screw 103.

The inside of the bellow 101 is adapted receive and eject body fluid. The body fluid enters the bellow via an inlet 109 when the bellow expands. The fluid exits the bellow 101 via an outlet 111 when the bellow is compressed. In order for the fluid to only enter the bellow via the inlet when the bellow expands, a valve 113 is provided to prevent fluid to enter via the outlet 111 during the expansion phase.

Similarly, the valve 113 is adapted to prevent fluid to exit via the inlet 109 when the bellow is compressed. The valve 113 is controlled by a control member 115 such as a solenoid.

The inlet and outlet are shaped to have tubes (not shown) fitted thereon. The tube connected to the inlet is preferably shaped and adapted to be placed in a treatment area from which body fluid is to be removed. The tube connected to the outlet is preferably shaped and adapted to be placed in a delivery area to which body fluid is to be moved from the treatment area.

During operation the device is adapted to compress the bellow in a compression phase during which fluid is ejected from the device 100 via the outlet tube to the delivery area for example by driving the motor to drive the screw. In a preferred embodiment a spring 117 is also compressed during the compression phase. During operation the device is further adapted to expand the bellow in an expansion phase during which fluid is sucked into the device 100 via the inlet tube from the treatment area for example by driving the screw in the opposite direction. In a preferred embodiment the spring 117 drives the bellow to expand during the expansion phase. When treating a patient the compression phase and expansion phase are continuously repeated whereby body fluid is removed from the treatment area to the delivery area.

In FIG. 286 the device 100 is shown as supplemented with a control unit 102 for controlling the operation of the device 100. The control unit 102 can receive and transmit signals for an external control unit 121. The control unit 121 is typically located outside the body when the device 100 is implanted inside a patient. In addition the device can be provided with a chargeable power source 123 connected to the motor. The power source 123 is adapted to receive wireless power from a second power source 125 which typically is located outside the patient when the implantable device 100 is implanted in a patient.

Hereby the power source 123 can be recharged at suitable time intervals thereby removing the need for replacing the power source.

In order to prevent or remove a possible occlusion in the tube the fluid movement device can be provided with a backward release member 126 adapted to generate a backward pressure of fluid or air in the tube for removing or preventing a possible occlusion in the tube. The backward pressure is preferably repeatedly according to a predetermined time schedule. In accordance with one embodiment the release member comprises a pre-pressurized reservoir of air and a valve adapted to release a puff of air in the tube. In accordance with another embodiment the device 100 is adapted to move fluid or air in the tube in the reversed direction thereby creating a reverse flow for prevent or remove a possible occlusion in the tube. This can for example be obtained by controlling the valve 113 to a reversed more of operating so that fluid exits the device 100 via the inlet. In accordance with yet another embodiment a reservoir of the drainage is pre-pressurized by the pump, and a valve of the device is adapted to release a puff of fluid or air in the tube extending from the pre-pressurized reservoir when the pressure has reached a predetermined level.

In FIG. 287a flowchart illustrating step performed when implanting the device 100 in a patient. First in a step 301 the skin is cut at locations corresponding to the location where the device is to be placed and where the tubes leading to and from the device are going to be placed. Next, in a step 303 the area from which body fluid is to be removed, the treatment area is dissected. Then, in a step 305, the area to which body fluid is to be moved, the delivery area, is dissected. Thereupon, in a step 307, the area where the device is to be placed, the placement area is dissected, if the placement area is different from the treatment area and the delivery area. Next, in a step 309 the device is placed in the placement area and the tubes extending between the device and the treatment area and the delivery area are put into place in steps 311 and 313, respectively.

In accordance with one embodiment a cleaning device 10 is inserted in the flow passageway from the treatment area to where the fluid is moved, I.e. the delivery area.

The design of a first preferred embodiment of a cleaning device 10 will now be described in detail, with reference to FIGS. 288-290. FIG. 288 shows a sectional view wherein the cleaning device 10 is provided in the flow passageway provided by a tube 2b. A filter 12 is provided across the flow passageway 14 formed in a housing 11 with the function of stopping particles brought forward in tube 2b by the flow, indicated by arrows in the figure. In this preferred embodiment, the filter 12 comprises a plurality of preferably equally spaced strips 12a of some suitable material, such as biocompatible metal or plastic. These strips 12a are preferably arranged mutual parallel.

The distance between two adjacent strips is small enough to stop any particles larger than some predetermined size. In accordance with one embodiment the distance is less than 2 millimeters, and even less than 1.0 millimeters. Also for some applications the distance could be larger. The flow passageway 14 can have an essentially square cross-sectional shape or it can take any suitable shape, such as rectangular or circular.

By providing a plurality of strips 12a as a filter across the flow passageway 14, a laminar flow is achieved downstream of the filter, which is can be advantageous. The flow configuration can be further enhanced by giving the plurality of strips 12a a desired cross-sectional shape, although the rectangular shape shown in FIG. 290 will be adequate for most purposes.

A first piston 16 is provided movable in a direction essentially perpendicular to the direction of the flow passageway 14, i.e., essentially perpendicular to the direction of the flow. This first piston 16 is driven by some suitable actuator means, such as pressurized air, a solenoid arrangement, an electrical servo motor or the like. A motor could be used to build up a stored power that could be released very fast, one example being a spring. In a preferred embodiment, pressurized air acts as the actuator means, since by latching the piston by means of a suitable latching means for the piston, building up the air pressure, and subsequently releasing the piston, very high speed of the piston is achieved, with enables short cleaning times of the filter.

The outer end portion of the first piston 16, i.e., the end portion facing the flow passageway 14, is essentially flush with the wall of the flow passageway in a non-active state of the cleaning device 10.

Also, the outer end portion is provided with a concave portion or recess 16a (exaggerated in the figures) in order to act as a particle capturing means, as will be explained below.

The strike range of the first piston 16 is preferably such that it extends all way across the flow passageway 14, as will be explained below with reference to FIGS. 291-294. A number of channels 16b corresponding to the number of strips 12a is provided in the first piston 16 to accommodate the strips when the first piston is in an extended position.

The first piston 16 is also provided with a plurality of through holes 17 in the direction of the flow passageway. These through holes will allow a flow through the flow passageway also during a cleaning operation, as will be explained below with reference to FIG. 295.

A second piston 18 is provided across the flow passageway 14 from the first piston 16. Also this second piston 18 is movable in a direction essentially perpendicular to the direction of the flow passageway 14 and is biased in the direction thereof by means of a spring 18a, for example. Likewise, the outer end portion of the second piston is provided with a recess 18b similar to the recess 16a of the first piston 16.

The first and second pistons 16, 18, are sealed to the housing 11 by means of a respective sealing 20, such as an O sealing.

A preferred embodiment of a cleaning method according to the invention will now be described with reference to FIGS. 291-294, showing different operational steps of the above-described device. FIG. 291 is a view similar to that of FIG. 288. However, this figure shows the cleaning device 10 during operation, wherein particles, generally designated 22, have assembled on the filter 12.

In FIG. 292, the first piston 16 has moved linearly from the retracted starting position shown FIG. 291 to an extended position, wherein the outer end portion thereof is in contact with the second piston 18. Due to the recess 16a in the outer end of the first piston 16, the particles 22 have been assembled in the recess 16a, whereby they have been brought with the first piston 16 during the movement thereof. In the step shown in FIG. 292, the particles are confined in the recess 16a between the first and second pistons 16, 18.

By moving the first piston 16 an additional distance from the position shown in FIG. 292, the second piston 18 is pushed against the force of the spring 18a to a fully retracted position, see FIG. 293.

The plurality of strips 12a is in this position fully received in a respective channel 16b in the first piston.

It is seen that the outer ends of the first and second pistons define an unobstructed cavity in which the particles are confined. It is thereby possible to remove these by some suitable means. One such means could be a third piston 24, which is movable in a direction perpendicular to both the direction of the flow passageway 14 and the direction of movement of the first and second pistons 16, 18. This third piston, the movement of which could be controlled by means of pressurized air, a solenoid, an electric motor etc., scrapes off the particles collected by the first piston 16 and moves them to a place outside of the cleaning device 10 and the flow passageway 14.

FIG. 295 shows a side view of the first piston 16 in a fully extended position, i.e., corresponding to the view of FIG. 294. It is here seen that in this position the through holes 17 will be aligned with the flow passageway 14, thereby allowing a flow therethrough also during cleaning of the filter 12.

FIG. 296 shows a cross-sectional view taken along line X-X of FIG. 294. It is here seen that the third piston 24 collects the particles 22 during a downward movement, indicated by an arrow in the figure. The particles are ejected from the cleaning device 10 when the third piston 24 has reached its lower end position, shown in FIG. 297.

Again with reference to FIG. 293, it will be realized that pressurized air can be used for ejecting the collected particles from the cavity formed by the first piston 16 and the second piston 18.

A cleaning system, generally designated 100 and comprising a cleaning device as described above will now be described with reference to FIGS. 667-679.

A cleaning system is shown in a more generalized block diagram form in FIG. 667, wherein the patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 1006 operable by polarized energy also is implanted in the patient for reversing the cleaning device 10. The wireless remote control of the external energy transmission device 200 transmits a wireless signal that carries polarized energy and the implanted energy transforming device 1002 transforms the wireless polarized energy into a polarized current for operating the electric switch 1006. When the polarity of the current is shifted by the implanted energy transforming device 1002 the electric switch 1006 reverses the function performed by the cleaning device 10.

FIG. 669 shows an embodiment identical to that of FIG. 667, except that an operation device 1007 implanted in the patient for regulating the cleaning device 10 is provided between the implanted energy transforming device 1002 and the cleaning device 10. This operation device can be in the form of a motor 1007, such as an electric servo motor. The motor 1007 is powered with energy from the implanted energy transforming device 1002, as the remote control of the external energy transmission device 200 transmits a wireless signal to the receiver of the implanted energy transforming device 1002.

FIG. 670 shows an embodiment identical to that of FIG. 667, except that it also comprises an operation device is in the form of an assembly 1008 including a motor/pump unit 78 and a fluid reservoir 1010 is implanted in the patient. In this case the cleaning device 10 is hydraulically operated, i.e. hydraulic fluid is pumped by the motor/pump unit 1009 from the fluid reservoir 1010 through a conduit 1011 to the cleaning device 10 to operate the cleaning device, and hydraulic fluid is pumped by the motor/pump unit 1009 back from the cleaning device 10 to the fluid reservoir 1010 to return the cleaning device to a starting position. The implanted energy transforming device 1002 transforms wireless energy into a current, for example a polarized current, for powering the motor/pump unit 1009 via an electric power supply line 1012.

Instead of a hydraulically operated cleaning device 10, it is also envisaged that the operation device comprises a pneumatic operation device. In this case, pressurized air can be used for regulation and the fluid reservoir is replaced by an air chamber and the fluid is replaced by air.

FIG. 671 shows an embodiment comprising the external energy transmission device 200 with its wireless remote control, the cleaning device 10, in this case hydraulically operated, and the implanted energy transforming device 1002, and further comprising a hydraulic fluid reservoir 1013, a motor/pump unit 1009 and an reversing device in the form of a hydraulic valve shifting device 1014, all implanted in the patient. The motor of the motor/pump unit 1009 is an electric motor. In response to a control signal from the wireless remote control of the external energy transmission device 200, the implanted energy transforming device 1002 powers the motor/pump unit 1009 with energy from the energy carried by the control signal, whereby the motor/pump unit 1009 distributes hydraulic fluid between the hydraulic fluid reservoir 1013 and the cleaning device 10. The remote control of the external energy transmission device 200 controls the hydraulic valve shifting device 1014 to shift the hydraulic fluid flow direction between one direction in which the fluid is pumped by the motor/pump unit 1009 from the hydraulic fluid reservoir 1013 to the cleaning device 10 to operate the cleaning device, and another opposite direction in which the fluid is pumped by the motor/pump unit 1009 back from the cleaning device 10 to the hydraulic fluid reservoir 1013 to return the cleaning device to a starting position.

FIG. 672 shows an embodiment identical to that of FIG. 667, except that an internal control unit 102 controlled by the wireless remote control of the external energy transmission device 200, an accumulator 1016 and a capacitor 1017 also are implanted in the patient. The internal control unit 102 arranges storage of electric energy received from the implanted energy transforming device 1002 in the accumulator 1016, which supplies energy to the cleaning device 10. In response to a control signal from the wireless remote control of the external energy transmission device 200, the internal control unit 102 either releases electric energy from the accumulator 1016 and transforms the released energy via power lines 1018 and 1019, or directly transforms electric energy from the implanted energy transforming device 1002 via a power line 1020, the capacitor 1017, which stabilizes the electric current, a power line 1021 and the power line 64, for the operation of the cleaning device 10.

The internal control unit is preferably programmable from outside the patient's body. In a preferred embodiment, the internal control unit is programmed to regulate the cleaning device 10 to remove any particles from the fluid movement device and place the particles outside the fluid movement device repeatedly according to a pre-programmed time-schedule.

In accordance with an alternative, the capacitor 1017 in the embodiment of FIG. 672 may be omitted. In accordance with another alternative, the accumulator 1016 in this embodiment may be omitted.

FIG. 673 shows an embodiment identical to that of FIG. 667, except that a battery 1022 for supplying energy for the operation of the cleaning device 10 and an electric switch 1023 for switching the operation of the cleaning device 10 also are implanted in the patient. The electric switch 1023 is operated by the energy supplied by the implanted energy transforming device 1002 to switch from an off mode, in which the battery 1022 is not in use, to an on mode, in which the battery 1022 supplies energy for the operation of the cleaning device 10.

FIG. 674 shows an embodiment identical to that of FIG. 673, except that an internal control unit 102 controllable by the wireless remote control of the external energy transmission device 200 also is implanted in the patient. In this case, the electric switch 1023 is operated by the energy supplied by the implanted energy transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the cleaning device 10.

FIG. 675 shows an embodiment identical to that of FIG. 674, except that an accumulator 1016 is substituted for the battery 1022 and the implanted components are interconnected differently. In this case, the accumulator 1016 stores energy from the implanted energy transforming device 1002. In response to a control signal from the wireless remote control of the external energy transmission device 200, the internal control unit 102 controls the electric switch 1023 to switch from an off mode, in which the accumulator 1016 is not in use, to an on mode, in which the accumulator 1016 supplies energy for the operation of the cleaning device 10.

FIG. 676 shows an embodiment identical to that of FIG. 675, except that a battery 1022 also is implanted in the patient and the implanted components are interconnected differently. In response to a control signal from the wireless remote control of the external energy transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the cleaning device 10.

Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the cleaning device 10.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the cleaning device 10 (mechanically operated).

FIG. 678 shows an embodiment identical to that of FIG. 676 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the cleaning device 10.

FIG. 679 schematically shows conceivable combinations of implanted components of the apparatus for achieving various communication options. Basically, there are the cleaning device 10, the internal control unit 102, motor/pump unit 1009, and the external energy transmission device 200 including the external wireless remote control. As already described above the wireless remote control transmits a control signal which is received by the internal control unit 102, which in turn controls the various implanted components of the apparatus.

A feedback device, preferably in the form of a sensor 1025, may be implanted in the patient for sensing a physical parameter of the patient, such as the pressure in a blood vessel. The internal control unit 102, or alternatively the external wireless remote control of the external energy transmission device 200, may control the cleaning device 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver. Alternatively, the wireless remote control may comprise a signal receiver or transceiver and the internal control unit 102 may comprise a signal transmitter or transceiver. The above transceivers, transmitters and receivers may be used for sending information or data related to the cleaning device 10 from inside the patient's body to the outside thereof.

Alternatively, the sensor 1025 may be arranged to sense a functional parameter of the cleaning device 10.

Where the motor/pump unit 1009 and battery 1022 for powering the motor/pump unit 1009 are implanted, the battery 1022 may be equipped with a transceiver for sending information on the condition of the battery 1022.

FIG. 680 shows an alternative embodiment wherein the cleaning device 10 is regulated from outside the patient's body. The cleaning system 100 comprises a cleaning device 10 connected to a battery 1022 via a subcutaneous switch 1026. Thus, the regulation of the cleaning device 10 is performed non-invasively by manually pressing the subcutaneous switch, whereby the operation of the cleaning device 10 is switched on and off. It will be appreciated that the shown embodiment is a simplification and that additional components, such as an internal control unit, can be added to the cleaning system.

FIG. 681 shows an alternative embodiment, wherein the cleaning system 100 comprises a cleaning device 10 in fluid connection with a hydraulic fluid reservoir 1013. Non-invasive regulation is performed by manually pressing the hydraulic reservoir connected to the cleaning device 10.

A further embodiment of a system according to the invention comprises a feedback device for sending information from inside the patient's body to the outside thereof to give feedback information related to at least one functional parameter of the clot removal device or system or a physical parameter of the patient, thereby optimizing the performance of the system.

One preferred functional parameter of the device is correlated to the transfer of energy for charging the internal energy source.

In FIG. 682, an arrangement is schematically illustrated for supplying an accurate amount of energy to a cleaning system 100 implanted in a patient, whose skin SK is indicated by a vertical line. A cleaning device 10 is connected to an implanted energy transforming device 1002, likewise located inside the patient, preferably just beneath the patient's skin SK. Generally speaking, the implanted energy transforming device 1002 may be placed in the abdomen, thorax, muscle fascia (e.g. in the abdominal wall), subcutaneously, or at any other suitable location. The implanted energy transforming device 1002 is adapted to receive wireless energy E transmitted from an external energy source 200a provided in the external energy transmission device 200 located outside the patient's skinSK in the vicinity of the implanted energy transforming device 1002.

As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy transforming device 1002. When an electric current is fed through the primary coil, energy in the form of a voltage is induced in the secondary coil which can be used to operate a cleaning device, e.g. after storing the incoming energy in an energy storing device or accumulator, such as a battery or a capacitor. However, the present invention is generally not limited to any particular energy transfer technique, TET devices or energy storing devices, and any kind of wireless energy may be used. Other energy transfer methods include but are not limited to non-induction methods such as by means of ultra-sonic devices or using light.

The amount of transferred energy can be regulated by means of an external control unit 34b controlling the external energy source 200a based on the determined energy balance, as described above.

In order to transfer the correct amount of energy, the energy balance and the required amount of energy can be determined by means of an internal control unit 102 connected to the cleaning device 10. The internal control unit 102 may thus be arranged to receive various measurements obtained by suitable sensors or the like, not shown, measuring certain characteristics of the cleaning device 10, reflecting the required amount of energy needed for proper operation of the cleaning device 10. Moreover, the current condition of the patient may also be detected by means of suitable measuring devices or sensors, in order to provide parameters reflecting the patient's condition. Hence, such characteristics and/or parameters may be related to the current state of the cleaning device 10, such as power consumption, operational mode and temperature, as well as the patient's condition reflected by, e.g., body temperature, blood pressure, heartbeats and breathing.

Furthermore, an energy storing device or accumulator 1016 may optionally be connected to the implanted energy transforming device 1002 for accumulating received energy for later use by the cleaning device 10. Alternatively or additionally, characteristics of such an accumulator, also reflecting the required amount of energy, may be measured as well. The accumulator may be replaced by a battery, and the measured characteristics may be related to the current state of the battery, such as voltage, temperature, etc. In order to provide sufficient voltage and current to the cleaning device 10, and also to avoid excessive heating, it is clearly understood that the battery should be charged optimally by receiving a correct amount of energy from the implanted energy transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 is adapted to determine the energy balance and/or the currently required amount of energy, (either energy per time unit or accumulated energy) based on measurements made by the above-mentioned sensors or measuring devices on the cleaning device 10, or the patient, or an energy storing device if used, or any combination thereof. The internal control unit 102 is further connected to an internal signal transmitter 82, arranged to transmit a control signal reflecting the determined required amount of energy, to an external signal receiver 34c connected to the external control unit 34b. The amount of energy transmitted from the external energy source 200a may then be regulated in response to the received control signal.

Alternatively, sensor measurements can be transmitted directly to the external control unit 34b wherein the energy balance and/or the currently required amount of energy can be determined by the external control unit 34b, thus integrating the above-described function of the internal control unit 102 in the external control unit 34b. In that case, the internal control unit 102 can be omitted and the sensor measurements are supplied directly to the internal signal transmitter 82 which sends the measurements over to the external signal receiver 34c and the external control unit 34b. The energy balance and the currently required amount of energy can then be determined by the external control unit 34b based on those sensor measurements.

Hence, feedback of information indicating the required energy can be used, which is more efficient because it is based on the actual use of energy that is compared to for example the received energy, e.g. with respect to the amount of energy, the energy difference, or the energy receiving rate as compared to the energy rate used by the cleaning device. The cleaning device may use the received energy either for consuming or for storing the energy in an energy storage device or the like. The different parameters discussed above would thus be used if relevant and needed and then as a tool for determining the actual energy balance. However, such parameters may also be needed per se for any actions taken internally to specifically operate the clot removal device.

The internal signal transmitter 82 and the external signal receiver 34c may be implemented as separate units using suitable signal transfer means, such as radio, IR (Infrared) or ultrasonic signals.

Alternatively, the internal signal transmitter 82 and the external signal receiver 34c may be integrated in the implanted energy transforming device 1002 and the external energy source 200a, respectively, so as to convey control signals in a reverse direction relative to the energy transfer, basically using the same transmission technique. The control signals may be modulated with respect to frequency, phase or amplitude.

The energy supply arrangement illustrated in FIG. 682 may operate basically in the following manner. The energy balance is first determined by the internal control unit 102. A control signal reflecting the required amount of energy is also created by the internal control unit 102, and the control signal is transmitted from the internal signal transmitter 82 to the external signal receiver 34c.

Alternatively, the energy balance can be determined by the external control unit 34b instead depending on the implementation, as mentioned above. In that case, the control signal may carry measurement results from various sensors. The amount of energy emitted from the external energy source 200a can then be regulated by the external control unit 34b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

A method is thus provided for controlling transmission of wireless energy supplied to an electrically operable cleaning device implanted in a patient. The wireless energy E is transmitted from an external energy source located outside the patient and is received by an internal energy receiver located inside the patient, the internal energy receiver being connected to the clot removal device for directly or indirectly supplying received energy thereto. An energy balance is determined between the energy received by the internal energy receiver and the energy used for the cleaning device. The transmission of wireless energy E from the external energy source is then controlled based on the determined energy balance.

A system is also provided for controlling transmission of wireless energy supplied to an electrically operable cleaning device implanted in a patient. The system is adapted to transmit the wireless energy E from an external energy source located outside the patient which is received by an implanted energy transforming device located inside the patient, the implanted energy transforming device being connected to the cleaning device for directly or indirectly supplying received energy thereto. The system is further adapted to determine an energy balance between the energy received by the implanted energy transforming device and the energy used for the cleaning device, and control the transmission of wireless energy E from the external energy source, based on the determined energy balance.

The functional parameter of the device is correlated to the transfer of energy for charging the internal energy source.

In yet an alternative embodiment, the external source of energy is controlled from outside the patient's body to release electromagnetic wireless energy, and released electromagnetic wireless energy is used for operating the cleaning device.

In another embodiment, the external source of energy is controlling from outside the patient's body to release non-magnetic wireless energy, and released non-magnetic wireless energy is used for operating the cleaning device.

Those skilled in the art will realize that the above various embodiments according to FIGS. 667-683 could be combined in many different ways. For example, the electric switch 1006 operated polarized energy could be incorporated in any of the embodiments of FIGS. 669, 672-678, the hydraulic valve shifting device 1014 could be incorporated in the embodiment of FIG. 670, and the gear box 1024 could be incorporated in the embodiment of FIG. 669.

Wireless transfer of energy for operating the cleaning device has been described to enable non-invasive operation. It will be appreciated that the cleaning device can be operated with wire bound energy as well. One such example is shown in FIG. 683, wherein an external switch 84 is interconnected between the external energy source 200a and an operation device, such as an electric motor regulating the cleaning device 10, by means of power lines 86 and 88. An external control unit 34b controls the operation of the external switch to effect proper operation of the cleaning device 10.

Also other filters can be used in the cleaning device 10. One such filter is depicted in FIG. 298.

The filter 90 in FIG. 298 comprises a rotating member 91 located in the flow passage way of the fluid movement device. The rotating member can be formed by a number of segments 92. Particles in the flow will caught by the segments and moved to the rim of the rotating member 91 where the particles can be effectively removed from the flow pathway of the fluid movement device. The cleaning device in FIG. 298 can be powered in the same manner as the cleaning device described above.

In FIG. 299 a general view of a patient having an implanted drainage system as described herein.

The system comprises a first end of the drainage system located in a treatment area 1. The system further comprises a pump 100 adapted to move fluid from the treatment area 1 to a delivery area 3. The treatment area can be any area from which fluid is to be move including but not limited to the abdomen, the lungs and the brain. Similarly the delivery area can be any suitable delivery area within the body, including but not limited to the urine bladder and the stomach.

The pump can be powered by an energy source 123 as described above. The energy source can be energized from outside the patient using a wireless energy transfer device. The energy transfer device can transfer energy in a way suitable such as by inductive energy using coils or ultra-sonic energy transfer or by transmitting light through the skin of the patient. Also the fluid passageway from the treatment area to the delivery area can comprise a cleaning device 10 as described above. The cleaning device can in one embodiment be powered by a motor and the motor can then be supplied with energy from the energy source 123.

In FIG. 300 the drainage system is shown in more detail. The view in FIG. 300 corresponds to the view in FIG. 299. However instead of showing the treatment area 1, FIG. 300 shows and end member 4 of the tube located in the treatment area. As described above the end member 4 can be designed differently for different treatment areas. Different end members are described in more detail below.

In FIGS. 301a-301d different exemplary designs of end members 4 are shown in more detail.

Thus, a connecting tube for use in an implantable fluid movement device being adapted to move body fluid from one part of the body, herein termed treatment area, of a human or mammal patient is provided.

A distal end of the connecting tube comprises in accordance with one embodiment a portion having a flat shape. Such an end portion can advantageously be used in the lungs when moving fluid from the lungs.

The end portion can have an essential circular shape as is shown in FIG. 301a or have a polygonal shape as is shown in FIG. 34b.

In accordance with one embodiment the distal end of the connecting tube can comprises a portion having a generally cylindrical shape as is shown in FIG. 301c. Such a shape can be preferred in applications where there is a risk that the tube end is sucked towards the wall of the treatment area. In

FIG. 301d yet another embodiment is shown with a very flexible tube end that can be used as a versatile tube in that it combines advantages of a flat tube end and a cylindrical tube end at the expense of the disadvantages of being flexible.

The tube ends are provided with holes or formed by a netlike structure. The diameter of the hole can in accordance with one embodiment be in the range of 1-10 mm. The number of holes and the diameter can typically depend on the treatment. As a general rule more holes and larger holes will give a lower sucking force and vice versa. Thus, areas where a low sucking force is required such as in the lungs can be treated using a tube end having many and large holes in the tube end.

In FIG. 302 a securing arrangement for securing a second end of a tube of the fluid movement device into the urine bladder is depicted. The arrangement comprises a tube end placed in the urine bladder 3 through a hole made in the wall of the urine bladder. On the outside the tube is led through a tunnel 95 formed by folding the outside wall of the urine bladder around the tube. The tunnel is secured around the tube by sutures 97 or similar. At the end of the tunnel a net structure 96 is tightly secured to the tube. The net structure has small diameter typically smaller than 0.5 mm. In any event the net structure has holes that will be small enough to be overgrown by tissue thereby providing a tight sealing so that no leakage occur. As stated above energy can be transferred in different manners from outside a patient into a implanted drain as described herein. In particular the energy can be transferred by means of an inductive energy transfer or by transmission using an ultrasonic energy transmission, or by transmission of energy using light. In FIG. 36a a triangle wave generator circuit which output is connected as an input terminal of an amplifier used for transmitting energy using an ultrasonic energy transmission.

In FIGS. 36a and 36b the symbols Y1, Y2, Y3 and so on symbolize test points within the circuit. The components in the circuit diagrams and their respective values are values that work in this particular implementation which of course is only one of an infinite number of possible design solutions.

FIG. 303a shows a circuit diagram containing most of an exemplary amplifier, in the lower left corner of FIG. 303a there is the LF input which is the input for the 25 kHz sine wave that should be amplified into a digital output signal. The LF-input there is the triangle wave input emanating from the Triangle schematic. To the right in the middle in the Core schematic there is the transmitting crystal, X4, connected to the differential digital outputs, positive and negative output, of the amplifier. The transmitting crystal X4 is in series with its associated tuning circuit components tuned to the sending frequency, which in this particular case is 25 kHz. FIGS. 303d-303e displays the relationship between the input and the output signal of the amplifier, in FIG. 36c Y25 is the input signal and Y2 is the positive digital output signal from the amplifier and in FIG. 303e Y13 is the negative digital output from the amplifier.

As described above the implanted drainage device can be powered by an internal power supply.

The same power supply or another power supply can be used to provide energy the filter and or cleaning device 10 as described herein. In FIG. 304 a general view similar to the view in FIG. 299 is shown where the filter and the cleaning device 10 is connected to a power supply. The apparatus in FIG. 304 comprises a first end of the drainage apparatus located in a treatment area 1. The apparatus further comprises a pump 100 adapted to move fluid from the treatment area 1 to a delivery area 3. The treatment area can be any area from which fluid is to be move including but not limited to the abdomen, the lungs and the brain. Similarly the delivery area can be any suitable delivery area within the body, including but not limited to the Urine bladder and the stomach. The apparatus can as stated above further comprise a filter and or a cleaning device 10. The filter and or cleaning device 10 can be powered by an energy source 123a as described above. The energy source can be the same as the energy source 123 powering a pump, but can also be another energy source. The energy source 123a can be energized from outside the patient using a wireless energy transfer device. The energy transfer device can transfer energy in a way suitable such as by inductive energy using coils or ultra-sonic energy transfer or by transmitting light through the skin of the patient. Also the fluid passageway from the treatment area to the delivery area can comprise a cleaning device 10 as described above. The cleaning device can in one embodiment be powered by a motor and the motor can then be supplied with energy from the energy source 123a.

In FIG. 305 the power supply to a filter and a cleaning device 10 is shown in more detail. The view in FIG. 305 corresponds to the view in FIG. 304. However instead of showing the treatment area 1,

FIG. 305 shows and end member 4 of the tube located in the treatment area. As is shown in FIG. 305 the energy source 123 and 123a can be energized from outside the skin SK of a patient by an external energy source 6. The energy source can also receive and transmit information to and from an external signaling device 7. The cleaning device can also be connected to changeable filter cassettes 127. In accordance with one embodiment a dirty filter of a cassette 127 is adapted to be replaced by a new filter of the cassette. The filter can also comprise a net structure.

In FIG. 306 a cassette 127 for holding filters is shown. The cassette 127 comprises a revolving cylinder 129 having segments 130 each holding a filter. The cylinder 129 is tightly sealed between two supports 131 holding the cylinder 129 in place and providing a tight sealing. The fluid passage way of an implantable drainage apparatus passes through the cassette 127. The cassette is driven by a motor 133 causing the cylinder 129 to revolve at suitable times. The motor is powered by a power supply 123b. The power supply can be a power supply like the power supplies 123 or 123a. In accordance with one embodiment the power supplies 123, 123a and 123b is the one and same power supply. As with the power supplies 123 and 123a, the power supply 123b can receive wireless energy in a suitable form, including but not limited to inductive energy ultrasonic energy, light energy or any other form of wireless energy set out above. The energy is supplied by an external wireless energy transmitter 6 adapted to transmit energy through the skin SK of a patient having the cassette 127 implanted. The power supply 123b can also comprise a control unit as described above for controlling the revolving cassette 127. The control unit can provide feedback to the outside and receive input data from an external transceiver 7 in a manner similar to the control unit used in conjunction with control of the pump.

In FIG. 307b the cassette 127 is shown from the side with the supports 131 and the revolving cylinder spaced apart is a disassembled view.

In FIG. 308 an alternative embodiment of the cassette 127 is shown. The view in FIG. 306 is similar to the view in FIG. 306. In the embodiment in FIG. 308 a magazine 135 having a number of cylinders 129 stored therein is provided. Hereby a cylinder 129 can by replaced by shifting the cylinders in the magazine 135. In one embodiment the cylinders are shifted by pressurized air.

In FIG. 309 the cassette 127 is shown from the side with the supports 131 and the revolving cylinder spaced apart is a disassembled view.

FIGS. 310-312 illustrate an embodiment using hydraulic treatment fluid according to the invention.

By reference to FIG. 310 and FIG. 311, the apparatus has an expandable member 220 with a cavity for accommodating hydraulic fluid that is placed inside the urinary bladder 230 which contains urine arrived from the ureters 232A, 232B. A control device 250 operates the expansion and thereby the volume of the expandable member. The control device 250 has a control assembly 21013 connected to a reservoir 254 for hydraulic fluid which is connected to the expandable member with an interconnecting device 256 for transporting hydraulic fluid between the reservoir 254 and the expandable member 220.

The interconnecting device 256 is a tube-shaped device surgically incised through the wall of the urinary bladder and attached thereto with tunneling technique whereby the bladder wall is sutured to itself. The interconnecting device is supported by the net 258 which seals fixates by admitting tissue in-growth. The control assembly 254 is located in the patient and includes a number functional elements necessary for operating the apparatus, such as an operating pump 259 for the hydraulic fluid, a source of energy for driving the operating pump and other energy consuming parts of the apparatus, and control functions including wireless communication with an external control unit. A suitable control assembly is described in more detail in the parallel patent application no. One or more parts of the control device may be implanted subcutaneously or in the abdominal cavity or the pelvic region or any other suitable place inside the body. The embodiment depicted in FIG. 311 is adapted for a patient suffering from a complication where the urinary sphincter is permanently closed. For this reason, the expandable member 220 of the apparatus needs to exert a considerable pressure (about 60-80 cm water pressure) to force urine out from the bladder and urine may thereby backflow through ureters 232A, 232B with potential risks for damaging the kidneys. To prevent from any such complications, the control device is provided with restriction devices 259A, 259B arranged to temporarily contract the ureters and close them during the operation of discharging urine with the expandable member. The restriction devices are operated from the control assembly in manner to perform their temporary contraction during the discharge performance. Suitable mechanical or hydraulically operated restriction devices and their control are described in more detail in European Patents Nos. EP 1253880; EP 1284691; and EP 1263355. The urine pressure in the ureter is normally around 50 cm water, however short term pressure increase is most likely not damaging the kidneys and therefore the restriction devices 259A and 259B may be omitted.

When the pump is not pumping to fill the expandable member and if the passage-way between the reservoir and the expandable member is free, then the expandable member may be emptied by urine filling the bladder. Another alternative is that the pump starts in steps to empty the expandable member for example pressure controlled or controlled by any other input sensor as mentioned elsewhere. In another embodiment a second connection may be established between the expandable member and the reservoir). If the pumping volume capacity is clearly much larger than the emptying capacity of the second connection this connection may always stand open.

By reference to FIG. 311 and FIG. 312, the apparatus in operated by activating the operating pump of the control assembly 254 which is operable in response from a signal from a remote control 253 The control assembly can also be connected to a pressure sensor 257 for monitoring the urinary pressure of the bladder. Several different type of input sensors may be used determining for example stretching or bending or pressure in the urine bladder wall or for example sensing volume or pressure inside the urine bladder. Most likely these sensors are only indirect causing the bladder to be emptied by presenting an alarm for the patient informing that it is time to empty the bladder. Such an alarm is generated audible or visually. The remote control may include a subcutaneous switch for controlling the emptying of the bladder or communicating via the body used as a wire or with wireless communication. The operating pump now transports hydraulic fluid from the reservoir 254, through the interconnection device 256 to the cavity of the expandable member 220, which thereby increases in volume in the urinary bladder and discharges urine through the urethra at a pressure that overcomes the closing force of the urethral sphincter, so voiding of the urinary bladder is accomplished. During this operation the control assembly operates to close restriction device 259A, 259B to prevent any urinary backflow in the ureters. When the discharging performance is finished and the operating pump is inactive, the restriction devices 259A, 259B are released so urine can refill the urinary bladder. By the pressure of the refilled urine, the expandable member 220 subsequently collapses to retain a shape as shown in FIG. 311 when ready for a new performance as monitored by the pressure sensor.

Some patients having urinary retention also have urinary incontinence. In such a case a separate urinary sphincter is included in the system, a restriction device closing the urethra until the patient wants to urinate. In such a case lower pressure is needed to empty the bladder because the no force would be needed to open the sphincter by intra bladder pressure. In this case the restriction devices 259A and 259B may be omitted.

The reservoir 254 may be placed anywhere inside the body, however preferable in the abdominal cavity, may be placed onto the urine bladder or in the pelvic region. The amount of liquid in the reservoir may be calibrated with fluid by using an injection port placed inside the body within reach from a special injection port needle. The reservoir may also be omitted and only the injection port may be used to fill and empty the expandable member. With the described embodiment it is also conceivable to control the duration/force of the urine discharge process, e.g. that data from the pressure sensor measuring the urinary pressure or easier the pressure inside the expandable member in the bladder controls the operation pump by logic in the control assembly. It should be noted that the expandable member may be elastic or only flexible, within the used pressure inside the same.

### H: Assisting the Pump Function of the Heart

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for assisting the pump function of the heart, examples of such devices for assisting the pump function of the heart will now be described.

The use of ceramic material is conceivable for entire device parts or parts exposed to wear, example of ceramic materials that can be used for this purpose is: zirconium ceramics or alumina ceramics, partially stabilized zirconia (PSZ), zirconium dioxide, titanium carbide, silicon carbide, sialons/silicon aluminum oxynitrides, boron nitride. The ceramic material could further comprise a hydroxy-apatite coating.

FIG. 313 shows an implantable medical device 100 for improving the pump function of the heart H of a human patient by applying an external force on the heart muscle. The implantable device 100 comprises a pump device 3 which comprises an operating device 57 that creates movement of a connecting arm 244 in contact with a heart contacting organ 2. The implantable device is adapted to be fixated to a structure of the human body comprising bone 240. The operating device and occasionally occurring other elements that requires control, are controlled from a control unit 102. The control unit 102 could comprise an injection port 910 for calibrating a fluid level of a hydraulic system, a battery 911 for supplying energy to the implantable device 1, a wireless transfer system 912 for transferring energy and/or information to or from the control unit from outside of the human body and at least one sensor 913 for sensing a variable of the implantable device 100 or the patient. The control unit communicates with the pump device 3 and other elements of the implantable device 100 through a connecting member 906. However it is also conceivable that the communication could be wireless.

FIG. 314 shows an implantable device 100 for improving the pump function of the heart H of a human patient by applying an external force on the heart muscle. The implantable device 100 comprises a pump device 3 which comprises an operating device 57 adapted to create a rotating movement through successive energizing coils 14 placed on a first plate 11 which is displaceable in relation to a second plate 12 comprising magnets 15. The magnetic field created between said coils 14 and said magnets 15 create a rotating movement of the second plate 12 in relation to the first plate 11. According to this embodiment the operating device is in connection with a first and second heart contacting organ 2a,b.

The first heart contacting organ 2a is attached to the second plate 12 and thereby moves in relation to the second heart contacting organ 2b which is fixedly attached to the pump device 3. The second heart contacting organ 2b serves as a dolly. The first and second heart contacting organs 2a,b exerts a force on the heart H from the left and right sides of the heart H which compresses the heart H and assist the pump function of the heart H.

FIG. 315 shows the implantable device 100 according to an embodiment where the pump device 3 is adapted to exert force on the heart H from the anterior A and posterior P side of the heart H. To enable the pump device 3 to exert force on the heart H from the anterior A and posterior P side of the heart H the implantable device 100 comprises a connecting arm 244 which attaches the pump device 3 to a fixating member 241a, which in turn is in contact with a first plate 242a, which is fixated to a second plate 242b of a second fixating member 241b located on the posterior side of a structure of the human body comprising bone 240. The first and second fixating members clamp the structure of the human body comprising bone 240 and thereby create the fixation of the implantable device 1. The first heart contacting organ 2a is attached to the second plate 12 and thereby moves in relation to the second heart contacting organ 2b which is fixedly attached to the pump device 3. The second heart contacting organ 2b serves as a dolly. The first and second heart contacting organs exerts a force on the heart H from the anterior A and posterior P sides of the heart H which compresses the heart H and assist the pump function of the heart H.

FIG. 316 shows the implantable device 100 in a lateral view where the operating device 57 comprising a first plate 11 comprising magnets 15, a second plate 12 comprising coils and a third plate 13 comprising magnets 15. The successive energizing of the coils 14 of the second plate 12 creates rotational movement of both the first and third plate by the magnetic contact created between the coils 14 and the magnets 15. The movement is transferred to the heart contacting organ 2 which in turn exerts force on the heart H.

FIG. 317 shows the implantable device 100 in a fontal view where the operating device 57 comprising a first plate 11 comprising magnets 15, a second plate 12 comprising coils and a third plate 13 comprising magnets 15. The successive energizing of the coils 14 of the second plate 12 creates rotational movement of both the first and third plate by the magnetic contact created between the coils 14 and the magnets 15. The first heart contacting organ 2a is fixated to the first plate 11, and the second heart contacting organ 2b is fixated to the third plate 13. The movement is transferred to the heart contacting organs 2a,b which in turn exerts force on the right and left sides of the heart H, which compresses the heart H and assist the pump function of the heart H.

FIG. 318 shows the implantable device 100 according to an embodiment where the pump device 3 is adapted to exert force on the heart H from the anterior A and posterior P side of the heart H. To enable the pump device 3 to exert force on the heart H from the anterior A and posterior P side of the heart H the implantable device 100 comprises a connecting arm 244 which attaches the pump device 3 to a fixating member 241a, which in turn is in contact with a first plate 242a, which is fixated to a second plate 242b of a second fixating member 241b located on the posterior side of a structure of the human body comprising bone 240. The first and second fixating members clamp the structure of the human body comprising bone 240 and thereby create the fixation of the implantable device 1. The first heart contacting organ 2a is fixated to the first plate, and the second heart contacting organ 2b is fixated to the third plate. The movement is transferred to the heart contacting organs 2a,b which in turn exerts force on the anterior A and posterior P sides of the heart H, which compresses the heart H and assist the pump function of the heart H.

FIG. 319 shows the operating device 57 is further detail wherein the operating device 57 comprises a first part comprising a plate 11 with a first surface, a second part comprising a second plate 12 having a second surface and a third part comprising a third plate 13 having a third surface. The first, second and third parts are displaceable in relation to each other and adapted for rotating movement. The second plate 12 comprises coils 14 whereas the first and third plate comprises magnets 15. The coils can be successively energized, controlled from a control unit 102, which creates movement of the first and third plates by the magnetic connection between the coils 14 and magnets 15. The surfaces of the first and second plate 11, 12 abut each other and is in substantially constant movement which hinders any growth of scar tissue that could interrupt the function of the operation device 57. To enable the operating device to resist the wear that constant movement of the abutting surfaces creates, the plates 11, 12, 13, or alternatively the surfaces, needs to be made of a highly durable material. Such a material could be a ceramic material, a carbon based material or a metallic material such as titanium or stainless steel. It is further conceivable that the plates or surfaces is made of a self-lubricating material such as a fluorpolymer, alternatively the surfaces could be adapted to be lubricated by means of an implantable lubricating system. The implantable lubricating system could be adapted to lubricate the plates 11, 12, 13 or surfaces with a biocompatible lubricating fluid such as hyaluronic acid. A combination of mentioned materials is further conceivable. The operating device 57 is according to the embodiment in FIG. 319 adapter for rotational movement, however it is possible that the operation device is adapted for reciprocating movement.

FIG. 320 shows the operating device 57 is further detail wherein the operating device 57 comprises a first part comprising a plate 11 with a first surface, a second part comprising a second plate 12 having a second surface and a third part comprising a third plate 13 having a third surface. The first, second and third parts are displaceable in relation to each other and adapted for rotational movement.

The second plate 12 comprises coils 14 whereas the first and third plate comprises magnets 15. The coils can be successively energized, controlled from a control unit 102, which creates movement of the first and third plates by the magnetic connection between the coils 14 and magnets 15. The operating device further comprises a center axis 17 which guides the rotational movement of the operating device 57.

FIG. 321 shows a lateral view of an embodiment where the implantable device 100 comprises a pump device 3. The pump device 3 comprises a piston 50 adapted for reciprocating movement placed in connection with an operating device 51 for operating the piston 50. The piston 50 is in turn in contact with a heart contacting organ 2 which in turn is in contact with the heart H of a human patient. The implantable device could in FIG. 321 further comprise a second pump device 53, the first and second pump devices are adapted to operate on the left and right side of the human heart H respectively, however in other embodiments the first and second pump devices 353 could be adapted to operate on the anterior and the posterior side of the heart H of a human patient. The implantable device 100 further comprises a first and second fixating member 241a, b adapted to fixate said implantable device 100 to a structure of the human body comprising bone 240. The fixating members comprises a first and second plate 242a, b which are fixated to each other using screws. To enable the pump device to resist the wear that constant movement of the abutting surfaces creates, affected parts or surfaces, needs to be made of a highly durable material. Such a material could be a ceramic material, a carbon based material or a metallic material such as titanium or stainless steel. It is further conceivable that parts or surfaces is made of a self-lubricating material such as a fluorpolymer, alternatively the surfaces could be adapted to be lubricated by means of an implantable lubricating system. The implantable lubricating system could be adapted to lubricate parts or surfaces with a biocompatible lubricating fluid such as hyaluronic acid. A combination of mentioned materials is further conceivable. The device is in substantially constant movement which hinders any growth of scar tissue that could interrupt the function of the device.

FIG. 322 shows a lateral view of an embodiment where the implantable device 100 is adapted for exerting force on the anterior and posterior side of the human heart H. The two heart contacting organs 2a, b are adapted to exert force on the heart H through the connection with the piston 50a adapted for reciprocating movement. According to this embodiment both the heart contacting organ 2a and the heart contacting organ 2b is hinged 52 to the pump device 3 which enables both heart contacting organs 2a, b to move and exert force on the heart H. To enable the heart contacting organs 2a,b to exert force on the heart H from the anterior and posterior side of the heart H the pump device 3 is attached to a connecting arm 244 which in turn is connected to the first fixating member 241a attached to the first plate 242a which is fixated to a structure of the human body comprising bone 240 through the connection with the second plate 242b of the second fixating member 241b. The piston 50a is according to this embodiment a piston adapted to create movement in two directions, which enables two heart contacting organs 2a, b to be operable by means of only one pump device 3. It is however conceivable that the piston 50a is of a type adapted to create movement in one direction 50b in which case two pump devices 353 could be provided to enable two heart contacting organs 2a, b to be operable.

FIG. 323 shows a frontal view of the implantable device 100 according to the embodiment shown in FIG. 317. The pump device 3 is here adapted to exert force on the heart H from the right and left side of the heart H through the heart contacting organs 2a, b hinged 52 to the pump device 3. The piston 50a is according to this embodiment a piston adapted to create movement in two directions, which enables two heart contacting organs 2a, b to be operable by means of only one pump device 3. It is however conceivable that the piston 50a is of a type adapted to create movement in one direction 50b in which case two pump devices 353 could be provided to enable two heart contacting organs 2a, b to be operable. According to this embodiment the first and second heart contacting organs 2a, b presses the heart towards each other which exerts a force on the heart H improving the pump function of the heart H.

FIG. 324 shows a frontal view of the implantable device 100 according to an embodiment where a piston 50b is adapted to create movement in one direction. According to this embodiment the second heart contacting organ 2b is hinged 52 to the implantable device 1, and the first heart contacting organ 2a is fixedly attached to the implantable device 1. According to this embodiment the second heart contacting organ 2b presses the heart towards the first heart contacting organ 2a which exerts a force on the heart H improving the pump function of the heart H.

FIG. 325 shows a lateral view of an embodiment where the implantable device 100 is adapted for exerting force on the anterior and posterior side of the human heart H. The second heart contacting organ 2b is hinged 52 to the implantable device 1, and the first heart contacting organ 2a is fixedly attached to the implantable device 1. The piston 50b is adapted to create movement in one direction and operates the second heart contacting organ 2b to exert force on the heart H from the anterior and posterior side of the heart through the second heart contacting organ 2b pressing the heart H against the first heart contacting organ 2a. To enable the exerting of force on the anterior and posterior side of the heart H the pump device 3 is attached to a connecting arm 244 which in turn is connected to the first fixating member 241a attached to the first plate 242a which is fixated to a structure of the human body comprising bone 240 through the connection with the second plate 242b of the second fixating member 241b.

FIG. 326 shows an embodiment where the implantable device 100 comprises a system for transferring of force from a remote location R to a distribution location DL. The heart contacting organ 2 is a section of the force distributing piston 50 which exerts force on the heart H, the force is transferred via a force transferring system 56, which could be a hydraulic, mechanic or pneumatic force transferring system 56. The force is created using an operating device 57, in this embodiment the operating device 57 is an electric motor, however it is also conceivable that motor is a hydraulic or pneumatic motor. The force generated by the operating device is then transferred to an eccentric member 58 which creates a reciprocal movement in a second piston 55. The reciprocating movement created in the second piston 55 it then transferred through the force transferring system 56 to the first piston 50 which is placed in reciprocating movement, and in turn exerts force on the heart H through the connection with the heart contacting organ 2. The first and second pistons 50, 55 are protected by a protective layer 54 which is made of a flexible material. The protective layer 54 hinders scar tissue to form in proximity to the moving parts, which could hinder the operation of the pistons 50, 55. The operating device 57 and additional parts of the system that could require control is controlled through the control unit 102, which in turn could be adapted to be wirelessly controlled from outside of the human body.

FIG. 327 shows an embodiment where the operating device 57 is an operating device adapted to create a rotating movement through successive energizing coils 14 placed on a first plate which is displaceable in relation to a second plate comprising magnets 15. The magnetic field created between said coils 14 and said magnets 15 creates a rotating movement of the second plate in relation to the first plate. A mechanical force transferring member 59 is attached to the second plate and hinged 60 to the piston 50. The piston in turn comprises the heart contacting organ 2 which exerts force on the heart H through the connection with the operating device 57. A control unit 102 for controlling the operating device is also provided, which in turn could be adapted to be wirelessly controlled from outside of the human body.

FIG. 328 shows an embodiment where the operating device 57 is a solenoid adapted to create a reciprocating movement of the piston 50 in connection with the heart contacting organ 2 to exert a force on the heart H of a human patient. A control unit 102 for controlling the operating device 57 is also provided, which in turn could be adapted to be wirelessly controlled from outside of the human body.

FIG. 329 shows, schematically, how a piston 50 housed in a protective layer 54 exerts force on the heart H of a human patient through the connection with a heart contacting organ 2. According to this embodiment the piston 50 is adapted to create reciprocating movement in two directions, the movement in the first direction is powered and the movement in the second direction could either be powered of created with a spring placed in relation to the piston 50.

FIG. 330 shows, schematically, how a piston 50 housed in a protective layer 54 exerts force on the heart H of a human patient through a mechanical force transferring system 59 which comprises a hinged joint 60. The mechanical force transferring system comprises a heart contacting organ 2 which in turn exerts force on the heart of a human patient H through the connection with the mechanical force transferring system 59 and the piston 50 adapted for reciprocating movement.

FIG. 331 shows, schematically, how two pistons 50a,b exerts force on the heart of a human patient H from the left and right side of the heart H. Each of the two pistons comprises a heart contacting organ 2a,b which exerts force on the heart H to compress the heart H to assist the pump function thereof.

According to other embodiments the two pistons 2a,b could be adapted to be placed on the anterior and posterior side of the heart H, or be movable to enable postoperative change in the position of the force exerted on the heart H.

FIG. 332 shows, schematically, how a piston 50 exerts force on the heart of a human patient through the connection with a heart contacting organ 2a from one side of the heart H. A second heart contacting organ 2b if fixedly attached to the implantable device 100 and serves as a dolly 61 to enable the implantable device 100 to exert force on the heart H.

FIG. 333 shows a frontal view of an implantable device 100 for improving the pump function of the heart of a human patient according to an embodiment wherein the implantable device comprises a pump device 3 comprises a rotating member 93 having a rotating center. A driving member 91 is attached to the rotating member 93 and adapted to perform an eccentric movement in relation to the rotating center of said rotating member 93. The driving member 91 is in contact with a heart contacting organ 2a,b which in turn is adapted to exert force on the heart H of a human patient. The pump device further comprises an operating device 57 for operating the driving member 91. The operating device is in connection with the rotating member through a force transferring member 92 which for example could be a band, cord or chain. The operating device 57 could be an electric, hydraulic or pneumatic motor, and could be adapted to be controlled from outside of the human body. To enable the pump device to resist the wear that constant movement of the abutting surfaces creates, affected parts or surfaces, needs to be made of a highly durable material. Such a material could be a ceramic material, a carbon based material or a metallic material such as titanium or stainless steel. It is further conceivable that parts or surfaces is made of a self-lubricating material such as a fluorpolymer, alternatively the surfaces could be adapted to be lubricated by means of an implantable lubricating system. The implantable lubricating system could be adapted to lubricate parts or surfaces with a biocompatible lubricating fluid such as hyaluronic acid. A combination of mentioned materials is further conceivable. The device is in substantially constant movement which hinders any growth of scar tissue that could interrupt the function of the device.

FIG. 334 shows a lateral view of an implantable device 100 for improving the pump function of the heart of a human patient according to an embodiment wherein the implantable device comprises a pump device 3 comprises a rotating member 93 having a rotating center. A driving member 91 is attached to the rotating member 93 and adapted to perform an eccentric movement in relation to the rotating center of said rotating member 93. The driving member 91 is in contact with a heart contacting organ 2a,b which in turn is adapted to exert force on the heart H of a human patient. The pump device further comprises an operating device 57 for operating the driving member 91. The operating device is in connection with the rotating member through a force transferring member 92 which for example could be a band, cord or chain. The operating device 57 could be an electric, hydraulic or pneumatic motor, and could be adapted to be controlled from outside of the human body. To enable the exerting of force on the anterior and posterior side of the heart H the pump device 3 is attached to a connecting arm 244 which in turn is connected to a fixating member 241 which is fixated to a structure of the human body comprising bone 240. According to this embodiment the first heart contacting organ is fixedly attached to the pump device 3 and serves as a dolly, whereas the second heart contacting organ is hinged to exert the force on the heart H.

FIG. 335 shows a lateral view of the implantable device 100 described in FIG. 333 where the pump device is adapted to exert force on the heart H from the right and left side of the heart H. The driving member 91 is in contact with an operating device 57.

FIG. 336 shows a frontal view of the pump device 3 wherein both the first heart contacting organ 2a and the second heart contacting organ 2b are hinged to the pump device 3 which enables the heart contacting organs 2a,b to exert force on the heart H, assisting the pump function thereof, from the right and left side of the heart H. The driving member 91 is according to this embodiment designed to operate two heart contacting organs 2a,b through the connection with the operating device 57.

FIG. 337 shows a lateral view of the pump device 3 wherein both the first heart contacting organ 2a and the second heart contacting organ 2b are hinged to the pump device 3, which enables the heart contacting organs 2a,b to exert force on the heart H, assisting the pump function thereof, from the anterior and posterior side the heart H. The driving member 91 is according to this embodiment designed to operate two heart contacting organs 2a,b through the connection with the operating device 57. To enable the exerting of force on the anterior and posterior side of the heart H the pump device 3 is attached to a connecting arm 244 which in turn is connected to a fixating member 241 which is fixated to a structure of the human body comprising bone 240.

FIG. 338 shows, schematically, an embodiment of a pump device according to any of the embodiments. An operating device 57 operates a rotating member 93 having a rotating center which is attached to a driving member 91 adapted to create an eccentric movement. The driving member is in contact with a pivot 100 which is hinged 101. The pivot could serve as a mechanical transmitter of force, or as a heart contacting organ 2 adapter to exert force on the heart H of a human patient. The operating device is controlled using a control unit 102 connected to the operating device through a connecting member 906. The operating device could be an electric, magnetic, hydraulic or pneumatic motor. In any embodiment where hydraulics is used an injection port 97 could be provided to enable the calibration of fluid in the hydraulic system. The control unit 102 could further comprise at least one sensor 98 for sensing a variable of the device, or the patient. Furthermore the control unit 102 could comprise a wireless transfer unit 99 for transferring of wireless energy and/or information. At least one battery 106 could also be provided in the control unit.

FIG. 339 shows, schematically, an embodiment of a pump device according to any of the embodiments. An operating device 57 operates a rotating member 93 having a rotating center which is attached to a driving member 91 adapted to create an eccentric movement. The driving member is in contact with a pivot 100 which is hinged 101 in one end, the other end is in contact with another pivot 103 which is hinged in its other end 107. The pivot system that the first and second pivot 100, 103 could be used as a mechanical transmitter of force, or said first or second pivot could comprise a heart contacting organ 2 adapted to exert force on the heart H.

FIG. 340 shows, schematically, an embodiment of a pump device 3, where the pump device 3 comprises a fixating member 241 which is adapted to fixate the pump device 3 to a structure of the human body comprising bone 240. The fixating member is adapted to fixate the pump device 3 to a structure of the human body comprising bone 240 using screws 243.

FIG. 341 shows, schematically, an embodiment of a pump device according to any of the embodiments. An operating device 57 operates a rotating member 93 having a rotating center which is attached to a driving member 91 adapted to create an eccentric movement. The driving member is in contact with a reciprocating member 104 which is guided by two guiding members 105a,b. The reciprocating member 104 could be used as a mechanical transmitter of force, or comprising a heart contacting organ 2 adapted to exert force on the heart H.

FIG. 342 shows a frontal view of a human patient according to an embodiment where the implanted device 100 is an LVAD 130 (Left Ventricular Assist Device). The LVAD can be fixated to a structure of the human body comprising bone 240 according to any of the embodiments described.

FIG. 343 shows a frontal view of a human patient according to an embodiment where the implanted device 100 is an artificial heart device 131. The artificial heart device 131 can be fixated to a structure of the human body comprising bone 240 according to any of the embodiments described.

FIG. 344 schematically shows a closed pneumatic or hydraulic implantable system for transferring force from a remote location R to a distribution location DL. The system comprises a first reservoir in the form of a first bellows 141 in contact with an operating device 57, which in this embodiment is an operating device comprising coils 14 and magnets 15, which is described in further detail previously. The volume of the first bellows 141 is affected by the contact with the operating device 57 which causes a fluid to be transferred in the fluid connection 142, which in turn affects the second bellows 140 on the distribution location DL. The second bellows could be used as a mechanical force transmitter or could be provided with a heart contacting organ 2 for exerting force on the heart of a human patient H. The implantable system is adapted to allow free flow of fluid between said first bellows 141 and said second bellows.

FIG. 345 schematically shows a closed pneumatic or hydraulic implantable system for transferring force from a remote location R to a distribution location DL. The system comprises a first reservoir in the form of a first piston 144. The volume in the cylinder 147 of the first piston 144 is affected by the contact with an operating device which causes a fluid to be transferred in the fluid connection 142, which in turn affects the second piston 143 on the distribution location DL, through the change of the fluid volume in the second cylinder 148. The second piston 143 could be used as a mechanical force transmitter or could be provided with a heart contacting organ 2 for exerting force on the heart of a human patient H. The implantable system is adapted to allow free flow of fluid between said first bellows 141 and said second bellows. The system could be adapted to operate using pressurized fluid in one direction and vacuum in the other direction, or pressurized fluid in both directions. It is also conceivable that the first and second pistons 143, 144 operates by means of a spring 145a, b in one direction.

FIG. 346 shows a frontal view of a patient where the remote location R of the implantable system for transferring force from a remote location R to a distribution location DL, is located in the abdominal region and the distribution location DL is located in connection with the heart H. The remote location comprises a control unit which in turn could comprise an operating device 146a, an injection port 146b, a battery 146c and at least one sensor 146d for sensing a variable of the implantable system or the patient.

FIG. 347 schematically shows a closed pneumatic or hydraulic implantable system for transferring force from a remote location R to a distribution location DL. The system comprises a first reservoir in the form of a first bellows 141 and a second reservoir in form of a second bellows 140. The first and second bellows are connected through a fluid connection 142. The fluid connection is adapted to always allow free flow of fluid between the first and second reservoir.

FIG. 348 schematically shows a closed pneumatic or hydraulic implantable system for transferring force from a remote location R to a distribution location DL. The system comprises a first reservoir in the form of a first bellows 141 and a second reservoir in form of a second bellows 140. The first and second bellows are connected through a fluid connection 142. The fluid connection is adapted to always allow free flow of fluid between the first and second reservoir. The system is operated using pressurized fluid in one direction and spring force from a spring 145 b in the second bellows in opposite direction.

FIG. 349 schematically shows a closed pneumatic or hydraulic implantable system for transferring force from a remote location R to a distribution location DL. The system comprises a first reservoir in the form of a first bellows 141 in contact with an operating device 57, which in this embodiment is an operating device comprising a rotating member 93 having a rotating center which is attached to a driving member 93 adapted to create an eccentric movement affecting the first bellows. The volume of the first bellows 141 is affected by the contact with the operating device 57 which causes a fluid to be transferred in the fluid connection 142, which in turn affects the second bellows 140 on the distribution location DL. The second bellows could be used as a mechanical force transmitter or could be provided with a heart contacting organ 2 for exerting force on the heart of a human patient H. The implantable system is adapted to allow free flow of fluid between said first bellows 141 and said second bellows 140.

A heart contacting organ 2, for example displayed in the embodiments above, could be adapted to change the position of the force exerted on the heart H of a human patient. This could be done by adjusting the position of the heart contacting organ 2 in relation to a fixating member 241 that fixates an implantable device 100 comprising the heart contacting organ 2 to a structure of the human body comprising bone 240. The adjustment could be performed by moving a connecting arm which is fixated to the fixating member 241 and the heart contacting organ 2. The object of moving the heart contacting organ 2 could be to increase the blood flow to area on which the heart contacting organ 2 exerts force. It could also be to improve the positioning of the heart contacting organ 2 such that the ability of the implantable device 100 to assist the pump function of the heart H. It could further be to relive the patient of any discomfort that the implantable device 100 might cause him/her.

FIG. 350 shows an embodiment in which the heart contacting organ 2 is attached to a connecting arm 244 in connection with the heart contacting organ 2 and the fixating member 241. The connecting arm 244 is hinged 170a,b to both the heart contacting organ 2 and the fixating member 241. However it is conceivable that the connecting arm 244 is hinged to one of the points 170a and 170b and fixedly attached to the other 170a,b respectively. The connecting arm 244 could be adapted to be operable either manually or powered. The connecting arm could be operable by means of an operation device 172 which could be an electric, a mechanical, a hydraulic or a pneumatic operating device 172. The operating device 172 could be placed in connection with the fixating member 241 and could be adapter to be remotely controlled from outside of the human body using a remote control. It is also conceivable that the connecting arm could be manually adjusted during a surgical or laparoscopic procedure in which case an adjusting member (not shown) could be provided to the implantable device 1. The adjusting member could be one that is adjustable by means of a surgical tool used in the surgical or laparoscopic procedure.

FIG. 351 shows an embodiment where the heart contacting organ 2 has been moved from the position in which it is placed in FIG. 350. The position of the force exerted on the heart H is thereby moved.

An alternative approach to moving the position of the force exerted on the heart is to move elements on the heart contacting organ 2. The elements could be pistons 173 and/or cushions 171 which could be electrically, mechanically, hydraulically or pneumatically operated. The pistons 173 and/or cushions 171 could be adapter to be remotely controlled from outside of the human body using a remote control. It is also conceivable that the pistons 173 and/or cushions 171 could be manually adjusted during a surgical or laparoscopic procedure. The heart contacting organ could comprise cushions 171 exclusively, pistons 173 exclusively or a mixture thereof.

FIG. 352 shows an embodiment in which multiple cushions 171 are placed on the heart contacting organ 2. The cushions 171 could be raised and lowered in relation to the heart contacting organ 2 to change the position of the force exerted on the heart H. FIG. 352 further shows a connecting arm 244 in connection with an operating device 172 for adjusting the location of the heart contacting organ 2 in relation to the heart H. The operating device 172 could be electrically, mechanically, hydraulically or pneumatically operated and could be adapter to be remotely controlled from outside of the human body using a remote control. It is also conceivable that the connecting arm 244 could be manually adjusted during a surgical or laparoscopic procedure. In the embodiment where the cushions 171 or pistons 173 are hydraulic or pneumatically operated the implantable device could further comprise a hydraulic or pneumatic system (not shown) for changing the volume of the cushion 171 or the volume under the piston 173, by moving a hydraulic or pneumatic fluid to or from the cushion 171.

FIG. 353 shows an embodiment where the heart contacting organ 2 comprises a cushion 174 that exerts force in the heart H. The cushion 174 can be moved on the heart contacting organ 2 to change the position of the force exerted on the heart H. According to this embodiment the heart contacting organ further comprises a rotational element 175 that rotates to create the movement of the cushion 174 on the great contacting organ 2. The rotational element could be operable manually, electrically, mechanically, hydraulically or pneumatically, and can further be adapted to be remotely controlled from outside of the human body using a remote control. FIG. 353 further shows a connecting arm 244 in connection with an operating device 172 for adjusting the location of the heart contacting organ 2 in relation to the heart H.

The operating device 172 could be electrically, mechanically, hydraulically or pneumatically operated and could be adapter to be remotely controlled from outside of the human body using a remote control.

FIG. 354 shows the embodiment according to FIG. 350 when implanted in a human body. The heart contacting organ 2 comprising cushions 171 and/or pistons 173 which could be raised and lowered in relation to the heart contacting organ to change the position of the force exerted on the heart H. The implantable device further comprises a connecting arm 244 in contact with the heart contacting organ 2 and an operating device 172 for operating the connecting arm 244. The operating device is in contact with the pate of the first fixating member 242a that together with the second fixating member 242b fixates the implantable device to a structure of the human body comprising bone 240. The implantable device further comprises a control unit 102 for controlling the heart pump device, the operating device 172 and the cushions 171 and/or pistons 173 placed on the heart contacting organ 2.

FIG. 355 shows an embodiment where the heart contacting organ 2 is operable to change the position of the force exerted on the heart H using two operating devices 177a,b the two operating devices could be mechanical, hydraulic or pneumatic devices. The heart contacting organ is operable through the connection with the operating device through the connecting arm 244 hinged to the heart contacting organ and the implantable device comprising the two operating devices 177a,b. According to other embodiments the connecting arm 244 is operable using only one operating device, in which case that operating device could be adapted for powered movement in two directions, or adapted for powered movement in one direction and spring loaded movement in the other direction.

FIG. 356 shows the heart H of a human patient H in a frontal view wherein 179 indicates the right ventricle which is a possible position for exerting force, and 178 indicates the left ventricle which also is a possible position for exerting force. It is also conceivable that force could be exerted on two different sides of the right 179 or left 178 ventricle, respectively.

FIG. 357 shows the implantable device 100 according to an embodiment where a pump device 3 is placed on an adjustment system comprising a first fixating member 241, a second fixating member 185 and a third fixating member 186. The first fixating member 241 is adapter for fixation in a structure of the human body comprising bone 240. The first fixating member comprises a first trench wherein the second fixating member 185 is adapted to move. The second fixating member 185 in turn comprises a second trench wherein the third fixating member 186 is adapted to move. The third fixating member 186 comprises a piston 182 which can be raised and lowered for adjusting the pump device 100 in a third axis. The third fixating member comprises a surface 183 to which the pump device 3 can be fixated.

Using said adjustment system the pump device 3 can be adjusted three dimensionally which can change the position of the force exerted on the heart H. The adjustment system can be operable by means of an implantable motor, the motor could be an electric, hydraulic or pneumatic motor. The motor could be adapted to be remotely controlled from outside of the human body using a remote control. The pump device 3 could hence be post-operatively adjusted by the patient or by a physician. The position of the pump device 3 could be verified from the outside of the human body using x-ray or ultra-sound.

FIG. 358 shows the adjustable system described in FIG. 357 in a second position.

The embodiments for changing the position of the force exerted on the heart H of a human patent described above could easily be combined with any of the embodiments of implantable devices described earlier.

FIG. 359-60 shows the fixation of an implantable device to a structure of the human body comprising bone 240. The structure could be the sternum, a part of the rib cage, comprising one or more ribs or a part of the vertebral column comprising at least one vertebra. According to one embodiment the implantable device 100 is fixated to the structure of the human body comprising bone 240 through a fixating member 241 said fixating member could comprise a plate 242 which is in contact with the structure of the human body comprising bone 240. The implantable device 100 could also be fixated to the structure of the human body comprising bone 240 using a second fixating member 241b which also could comprise a plate 242b in which in turn could be in contact with the structure of the human body comprising bone 240.

FIG. 359 shows an embodiment where the implantable device 100 is fixated to a structure of the human body comprising bone 240. The structure could be the sternum, a part of the rib cage comprising one or more ribs or a part of the vertebral column structure comprising at least one vertebra. According to the embodiment the implantable device 100 comprises a first fixating member 241a comprising a plate 242a and a second fixating member 241b comprising a plate 242b. The first and second fixating members are attached to each other using through-going screws 243 placed from the anterior side A of the structure of the human body comprising bone 240. An alternative embodiment could comprise screws placed from the posterior side P of the structure of the human body comprising bone 240. The first fixating member 241a and the second fixating member 241b clamp the structure of the human body comprising bone 240. The fixating member 241a could be in contact with a connecting arm 244 which in turn could be in contact with a heart pump device.

FIG. 360 shows an embodiment where the implantable device 100 is fixated to a structure of the human body comprising bone 240 using only one fixating member 241a comprising a plate 242a. The structure could be the sternum, a part of the rib cage comprising one or more ribs or a part of the vertebral column structure comprising at least one vertebra. Through-going screws 243 is placed form the anterior side A the structure of the human body comprising bone 240 and fixated in the plate 242a.

An alternative embodiment could comprise screws placed from the posterior side P of the structure of the human body comprising bone 240 in which case the screws could be fixated in nuts placed in connection with the structure of the human body comprising bone, or fixated in directly in the bone of the structure of the human body comprising bone 240. The fixating member 241a could be in contact with a connecting arm 244 which in turn could be in contact with a heart pump device.

FIG. 361 shows an embodiment where the implantable device 100 is fixated to a structure of the human body comprising bone 240. The structure could be the sternum, a part of the rib cage comprising one or more ribs or a part of the vertebral column comprising at least one vertebra. According to the embodiment the implantable device 100 comprises a first fixating member 241a comprising a plate 242a and a second fixating member 241b comprising a plate 242b. The first and second fixating members are attached to each other using through-going screws 243 placed from the posterior side P of the structure of the human body comprising bone 240. The screws are fixated to nuts 245 placed on the anterior side of the structure comprising bone 240. An alternative embodiment could comprise screws placed from the anterior side A of the structure of the human body comprising bone 240, in which case the nuts is placed on the posterior side P of the structure comprising bone 240. The first fixating member 241a and the second fixating member 241b clamp the structure of the human body comprising bone 240. The fixating member 241a could be in contact with a connecting arm 244 which in turn could be in contact with a heart pump device.

FIG. 362 shows an embodiment where the implantable device 100 is fixated to a structure of the human body comprising bone 240 using only one fixating member 241a comprising a plate 242a. The structure could be the sternum, a part of the rib cage comprising one or more ribs or a part of the vertebral column structure comprising at least one vertebra. Screws 243 that fixates the fixating member to the structure of the human body comprising bone is placed form the posterior side P the structure of the human body comprising bone 240. The screws fixate the fixating member to both the posterior and the anterior cortex of the structure of the human body comprising bone 240, however it is conceivable that the screws are fixated only to the anterior or posterior cortex. An alternative embodiment could comprise screws placed from the anterior side a of the structure of the human body comprising bone 240, in which case the fixating member 241a is placed on the anterior side A of the structure of the human body comprising bone 240.

FIG. 363 shows an embodiment where the implantable device 100 is fixated to a structure of the human body comprising bone 240 using one fixating member 241b comprising a plate 242b, and one fixating member 241a without a plate. The structure could be the sternum, a part of the rib cage comprising one or more ribs or a part of the vertebral column structure comprising at least one vertebra. Screws 243 that fixates the fixating members 241a,b to the structure of the human body comprising bone 240 is placed form the anterior side A of the structure of the human body comprising bone 240 and fixated in the fixating member 241a. The first fixating member 241a and the second fixating member 241b clamp the structure of the human body comprising bone 240. The fixating member 241a could be in contact with a connecting arm 244 which in turn could be in contact with a heart pump device.

FIG. 364 shows an embodiment where the implantable device 100 is fixated to a structure of the human body comprising bone 240 using one fixating member 241b comprising a plate 242b, and one fixating member 241a without a plate. The structure could be the sternum, a part of the rib cage comprising one or more ribs or a part of the vertebral column structure comprising at least one vertebra.

Screws 243 that fixates the fixating members 241a,b to the structure of the human body comprising bone 240 is placed form the posterior side P of the structure of the human body comprising bone 240 and fixated in the plate 242b of the fixating member 241b. The first fixating member 241a and the second fixating member 241b clamp the structure of the human body comprising bone 240. The fixating member 241a could be in contact with a connecting arm 244 which in turn could be in contact with a heart pump device.

FIG. 365 shows an embodiment where the implantable device 100 is adapted to be fixated to the sternum 250 of a human patient. The device is fixated using a fixating member 241b which is fixated to the sternum using screws 243. However the implantable device could be fixated to the sternum 250 of a human patent using any of the ways to place the fixating members described previously.

FIG. 366 shows an embodiment where the implantable device 100 is adapted to be fixated to two ribs 251, 252. A fixating member 241 comprising a plate 242b is fixated with screws adapted to fixate the fixating member to the cortex of the ribs.

FIG. 367 shows an embodiment where the implantable device 100 is adapted to be fixated to two ribs 251, 252. A first plate 242a is provided on the posterior side of the rib cage, whereas a second plate 242b is provided in the anterior side of the rib cage. Screws 243 penetrate the ribs and fixates the first plate 242a to the second plate 242b. The tightening of the screws creates a clamping effect of the ribs 251, 252 and provides the fixation of the implantable device 1. In another embodiment (not shown) he screws 243 are placed between the ribs 251, 252 and that ways provides a clamping effect of the ribs 251, 252.

FIG. 368 shows an embodiment where the implantable device 100 is adapted to be fixated to one rib 252. A plate 242a is provided on the posterior side of the rib cage and screws 243 are provided from the outside thereof, penetrating the rib 252 and fixating the plate 242a to the rib 252.

FIG. 369 shows an embodiment where the implantable device 100 is adapted to be fixated to one rib 252 using cord or band 254, this way there is no need to penetrate the rib 252. However the implantable device could be fixated to the ribcage of a human patent using any of the ways to place the fixating members described previously.

FIG. 370 shows an embodiment where the implantable device 100 is adapted to be fixated to a vertebra 255 of the vertebral column. A fixating member 241 is fixated to the vertebra 255 using screws 243. The implantable device further comprises a connecting arm 244 that connects the implantable device 100 to the fixating member 241.

FIG. 371 shows an embodiment where the implantable device 100 is adapted to be fixated to two vertebras 255, 256 of the vertebral column. A fixating member 241 is fixated to the two vertebras 255, 256 using screws 243. The implantable device further comprises a connecting arm 244 that connects the implantable device 100 to the fixating member 241.

FIG. 372 shows an embodiment where the implantable device is adapted to be fixated to a vertebra 255 of the vertebral column by clamping said vertebra 255. Two fixating members 241a, 241b is placed on two sides of the vertebra and an attachment comprising screws 243 clamps the vertebra between the first and second fixating members 241a,b. The implantable device further comprises a connecting arm 244 that connects the implantable device 100 to the fixating member 241.

In all of the above mentioned embodiments the means of attachment could be replaced with other mechanical attachments or an adhesive. Other mechanical attachments suitable could be: pop-rivets, nails, staples, band or cord. The mechanical fixating members could be of a metallic or ceramic material. Suitable metallic materials could be titanium or surgical steel.

FIG. 373 shows an embodiment where the heart contacting organ 2 is adapted to compress the heart H to assist the pump function thereof. A stimulation device 907 is attached to the heart contacting organ 2 and is adapted to stimulate the heart H to achieve an additional assistance of said pump function after the heart contacting organ 2 has placed the heart in the compressed state. According to an embodiment the heart contacting organ is attached to a connecting arm 244 which in turn is attached to a mechanical, electrical or hydraulic operating device 172 which operates the heart contacting organ 2. The operating device 172 is in turn attached a fixating member which fixates the device to a structure of the human body comprising bone 244 using mechanical fixating members such as screws, or adhesive. A control unit 102 for controlling the operating device 172 in accordance with any of the embodiments described in this application is in connection with said operating device 172 though a connecting member 906. However it is also conceivable that the control unit 102 communicates wirelessly with the operating device 172 as an external control unit 200.

FIG. 666 illustrates a system for treating a disease comprising an apparatus 10 placed in the abdomen of a patient. An implanted energy-transforming device 1002 is adapted to supply energy consuming components of the apparatus with energy via a power supply line 1003. An external energy-transmission device 200 for non-invasively energizing the apparatus 10 transmits energy by at least one wireless energy signal. The implanted energy-transforming device 1002 transforms energy from the wireless energy signal into electric energy which is supplied via the power supply line 1003.

The implanted energy-transforming device 1002 may also comprise other components, such as: a coil for reception and/or transmission of signals and energy, an antenna for reception and/or transmission of signals, a microcontroller, a charge control unit, optionally comprising an energy storage, such as a capacitor, one or more sensors, such as temperature sensor, pressure sensor, position sensor, motion sensor etc., a transceiver, a motor, optionally including a motor controller, a pump, and other parts for controlling the operation of a medical implant.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field.

The wireless energy-transmission device 200 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal.

Generally speaking, the energy-transforming device 1002 is provided for transforming wireless energy of a first form transmitted by the energy-transmission device 200 into energy of a second form, which typically is different from the energy of the first form. The implanted apparatus 10 is operable in response to the energy of the second form. The energy-transforming device 1002 may directly power the apparatus with the second form energy, as the energy-transforming device 1002 transforms the first form energy transmitted by the energy-transmission device 200 into the second form energy. The system may further include an implantable accumulator, wherein the second form energy is used at least partly to charge the accumulator.

Alternatively, the wireless energy transmitted by the energy-transmission device 200 may be used to directly power the apparatus, as the wireless energy is being transmitted by the energy-transmission device 200. Where the system comprises an operation device for operating the apparatus, as will be described below, the wireless energy transmitted by the energy-transmission device 200 may be used to directly power the operation device to create kinetic energy for the operation of the apparatus.

The wireless energy of the first form may comprise sound waves and the energy-transforming device 1002 may include a piezo-electric element for transforming the sound waves into electric energy.

The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus.

Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus.

The external energy-transmission device 200 also includes a wireless remote control having an external signal transmitter for transmitting a wireless control signal for non-invasively controlling the apparatus. The control signal is received by an implanted signal receiver which may be incorporated in the implanted energy-transforming device 1002 or be separate there from.

The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field.

The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals.

FIG. 667 illustrates the system of FIG. 666 in the form of a more generalized block diagram showing the apparatus 10, the energy-transforming device 1002 powering the apparatus 10 via power supply line 1003, and the external energy-transmission device 200, The patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 1006 operable for example by polarized energy also is implanted in the patient for reversing the apparatus 10. When the switch is operated by polarized energy the wireless remote control of the external energy-transmission device 200 transmits a wireless signal that carries polarized energy and the implanted energy-transforming device 1002 transforms the wireless polarized energy into a polarized current for operating the electric switch 1006. When the polarity of the current is shifted by the implanted energy-transforming device 1002 the electric switch 1006 reverses the function performed by the apparatus 10.

FIG. 669 shows an embodiment identical to that of FIG. 667, except that an operation device 1007 implanted in the patient for operating the apparatus 10 is provided between the implanted energy-transforming device 1002 and the apparatus 10. This operation device can be in the form of a motor 1007, such as an electric servomotor. The motor 1007 is powered with energy from the implanted energy-transforming device 1002, as the remote control of the external energy-transmission device 200 transmits a wireless signal to the receiver of the implanted energy-transforming device 1002.

FIG. 670 shows an embodiment identical to that of FIG. 667, except that it also comprises an operation device is in the form of an assembly 1008 including a motor/pump unit 1009 and a fluid reservoir 1010 is implanted in the patient. In this case the apparatus 10 is hydraulically operated, i.e. hydraulic fluid is pumped by the motor/pump unit 1009 from the fluid reservoir 1010 through a conduit 1011 to the apparatus 10 to operate the apparatus, and hydraulic fluid is pumped by the motor/pump unit 1009 back from the apparatus 10 to the fluid reservoir 1010 to return the apparatus to a starting position.

The implanted energy-transforming device 1002 transforms wireless energy into a current, for example a polarized current, for powering the motor/pump unit 1009 via an electric power supply line 1012.

Instead of a hydraulically operated apparatus 10, it is also envisaged that the operation device comprises a pneumatic operation device. In this case, the hydraulic fluid can be pressurized air to be used for regulation and the fluid reservoir is replaced by an air chamber.

In all of these embodiments the energy-transforming device 1002 may include a rechargeable accumulator like a battery or a capacitor to be charged by the wireless energy and supplies energy for any energy consuming part of the system.

As an alternative, the wireless remote control described above may be replaced by manual control of any implanted part to make contact with by the patient's hand most likely indirect, for example a press button placed under the skin.

FIG. 671 shows an embodiment comprising the external energy-transmission device 200 with its wireless remote control, the apparatus 10, in this case hydraulically operated, and the implanted energy-transforming device 1002, and further comprising a hydraulic fluid reservoir 1013, a motor/pump unit 1009 and an reversing device in the form of a hydraulic valve shifting device 1014, all implanted in the patient. Of course the hydraulic operation could easily be performed by just changing the pumping direction and the hydraulic valve may therefore be omitted. The remote control may be a device separated from the external energy-transmission device or included in the same. The motor of the motor/pump unit 1009 is an electric motor. In response to a control signal from the wireless remote control of the external energy-transmission device 200, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 10. The remote control of the external energy-transmission device 200 controls the hydraulic valve shifting device 1014 to shift the hydraulic fluid flow direction between one direction in which the fluid is pumped by the motor/pump unit 1009 from the hydraulic fluid reservoir 1013 to the apparatus 10 to operate the apparatus, and another opposite direction in which the fluid is pumped by the motor/pump unit 1009 back from the apparatus 10 to the hydraulic fluid reservoir 1013 to return the apparatus to a starting position.

FIG. 672 shows an embodiment comprising the external energy-transmission device 200 with its wireless remote control, the apparatus 10, the implanted energy-transforming device 1002, an implanted internal control unit 102 controlled by the wireless remote control of the external energy-transmission device 200, an implanted accumulator 1016 and an implanted capacitor 1017. The internal control unit 102 arranges storage of electric energy received from the implanted energy-transforming device 1002 in the accumulator 1016, which supplies energy to the apparatus 10. In response to a control signal from the wireless remote control of the external energy-transmission device 200, the internal control unit 102 either releases electric energy from the accumulator 1016 and transfers the released energy via power lines 1018 and 1019, or directly transfers electric energy from the implanted energy-transforming device 1002 via a power line 1020, the capacitor 1017, which stabilizes the electric current, a power line 1021 and the power line 1019, for the operation of the apparatus 10.

The internal control unit is preferably programmable from outside the patient's body. In a preferred embodiment, the internal control unit is programmed to regulate the apparatus 10 according to a pre-programmed time-schedule or to input from any sensor sensing any possible physical parameter of the patient or any functional parameter of the system.

In accordance with an alternative, the capacitor 1017 in the embodiment of FIG. 319 may be omitted. In accordance with another alternative, the accumulator 1016 in this embodiment may be omitted.

FIG. 673 shows an embodiment identical to that of FIG. 667, except that a battery 1022 for supplying energy for the operation of the apparatus 10 and an electric switch 1023 for switching the operation of the apparatus 10 also are implanted in the patient. The electric switch 1023 may be controlled by the remote control and may also be operated by the energy supplied by the implanted energy-transforming device 1002 to switch from an off mode, in which the battery 1022 is not in use, to an on mode, in which the battery 1022 supplies energy for the operation of the apparatus 10.

FIG. 674 shows an embodiment identical to that of FIG. 673, except that an internal control unit 102 controllable by the wireless remote control of the external energy-transmission device 200 also is implanted in the patient. In this case, the electric switch 1023 is operated by the energy supplied by the implanted energy-transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the apparatus 10.

FIG. 675 shows an embodiment identical to that of FIG. 674, except that an accumulator 1016 is substituted for the battery 1022 and the implanted components are interconnected differently. In this case, the accumulator 1016 stores energy from the implanted energy-transforming device 1002. In response to a control signal from the wireless remote control of the external energy-transmission device 200, the internal control unit 102 controls the electric switch 1023 to switch from an off mode, in which the accumulator 1016 is not in use, to an on mode, in which the accumulator 1016 supplies energy for the operation of the apparatus 10. The accumulator may be combined with or replaced by a capacitor.

FIG. 676 shows an embodiment identical to that of FIG. 675, except that a battery 1022 also is implanted in the patient and the implanted components are interconnected differently. In response to a control signal from the wireless remote control of the external energy-transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the apparatus 10.

Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the apparatus 10.

It should be understood that the switch 1023 and all other switches in this application should be interpreted in its broadest embodiment. This means a transistor, MCU, MCPU, ASIC, FPGA or a DA converter or any other electronic component or circuit that may switch the power on and off. Preferably the switch is controlled from outside the body, or alternatively by an implanted internal control unit.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the apparatus 10 (mechanically operated). Even simpler is to switch the direction of the motor electronically. The gear box interpreted in its broadest embodiment may stand for a servo arrangement saving force for the operation device in favor of longer stroke to act.

FIG. 678 shows an embodiment identical to that of FIG. 677 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the apparatus 10.

FIG. 679 schematically shows conceivable combinations of implanted components of the apparatus for achieving various communication options. Basically, there are the apparatus 10, the internal control unit 102, motor or pump unit 1009, and the external energy-transmission device 200 including the external wireless remote control. As already described above the wireless remote control transmits a control signal which is received by the internal control unit 102, which in turn controls the various implanted components of the apparatus.

A feedback device, preferably comprising a sensor or measuring device 1025, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow.

The feedback may be sent to the internal control unit or out to an external control unit preferably via the internal control unit. Feedback may be sent out from the body via the energy transfer system or a separate communication system with receiver and transmitters.

The internal control unit 102, or alternatively the external wireless remote control of the external energy-transmission device 200, may control the apparatus 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver. Alternatively, the wireless remote control may comprise a signal receiver or transceiver and the internal control unit 102 may comprise a signal transmitter or transceiver. The above transceivers, transmitters and receivers may be used for sending information or data related to the apparatus 10 from inside the patient's body to the outside thereof.

Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly.

FIG. 680 shows an alternative embodiment wherein the apparatus 10 is regulated from outside the patient's body. The system 100 comprises a battery 1022 connected to the apparatus 10 via a subcutaneous electric switch 1026. Thus, the regulation of the apparatus 10 is performed non-invasively by manually pressing the subcutaneous switch, whereby the operation of the apparatus 10 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 681 shows an alternative embodiment, wherein the system 100 comprises a hydraulic fluid reservoir 1013 hydraulically connected to the apparatus. Non-invasive regulation is performed by manually pressing the hydraulic reservoir connected to the apparatus.

The system may include an external data communicator and an implantable internal data communicator communicating with the external data communicator. The internal communicator feeds data related to the apparatus or the patient to the external data communicator and/or the external data communicator feeds data to the internal data communicator.

FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 1002 connected to implanted energy consuming components of the apparatus 10. Such an energy receiver 1002 may include an energy source and/or an energy-transforming device. Briefly described, wireless energy is transmitted from an external energy source 200a located outside the patient and is received by the internal energy receiver 1002 located inside the patient. The internal energy receiver is adapted to directly or indirectly supply received energy to the energy consuming components of the apparatus 10 via a switch 1026. An energy balance is determined between the energy received by the internal energy receiver 1002 and the energy used for the apparatus 10, and the transmission of wireless energy is then controlled based on the determined energy balance. The energy balance thus provides an accurate indication of the correct amount of energy needed, which is sufficient to operate the apparatus 10 properly, but without causing undue temperature rise.

In FIG. 682 the patient's skin is indicated by a vertical line SK. Here, the energy receiver comprises an energy-transforming device 1002 located inside the patient, preferably just beneath the patient's skin SK. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy-source 200a provided in an external energy-transmission device 200 located outside the patient's skin SK in the vicinity of the implanted energy-transforming device 1002.

As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used.

The amount of energy received by the implanted energy receiver may be compared with the energy used by the implanted components of the apparatus. The term “energy used” is then understood to include also energy stored by implanted components of the apparatus. A control device includes an external control unit 200b that controls the external energy source 200a based on the determined energy balance to regulate the amount of transferred energy. In order to transfer the correct amount of energy, the energy balance and the required amount of energy is determined by means of a determination device including an implanted internal control unit 102 connected between the switch 1026 and the apparatus 10. The internal control unit 102 may thus be arranged to receive various measurements obtained by suitable sensors or the like, not shown, measuring certain characteristics of the apparatus 10, somehow reflecting the required amount of energy needed for proper operation of the apparatus 10. Moreover, the current condition of the patient may also be detected by means of suitable measuring devices or sensors, in order to provide parameters reflecting the patient's condition. Hence, such characteristics and/or parameters may be related to the current state of the apparatus 10, such as power consumption, operational mode and temperature, as well as the patient's condition reflected by parameters such as; body temperature, blood pressure, heartbeats and breathing. Other kinds of physical parameters of the patient and functional parameters of the device are described elsewhere.

Furthermore, an energy source in the form of an accumulator 1016 may optionally be connected to the implanted energy-transforming device 1002 via the control unit 102 for accumulating received energy for later use by the apparatus 10. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 10, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 of the determination device is adapted to determine the energy balance and/or the currently required amount of energy, (either energy per time unit or accumulated energy) based on measurements made by the above-mentioned sensors or measuring devices of the apparatus 10, or the patient, or an implanted energy source if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal.

Alternatively, the determination device may include the external control unit 200b. In this alternative, sensor measurements can be transmitted directly to the external control unit 200b wherein the energy balance and/or the currently required amount of energy can be determined by the external control unit 200b, thus integrating the above-described function of the internal control unit 102 in the external control unit 200b. In that case, the internal control unit 102 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements.

Hence, the present solution according to the arrangement of FIG. 682 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus.

The internal signal transmitter 1027 and the external signal receiver 200c may be implemented as separate units using suitable signal transfer means, such as radio, IR (Infrared) or ultrasonic signals.

Alternatively, the internal signal transmitter 1027 and the external signal receiver 200c may be integrated in the implanted energy-transforming device 1002 and the external energy source 200a, respectively, so as to convey control signals in a reverse direction relative to the energy transfer, basically using the same transmission technique. The control signals may be modulated with respect to frequency, phase or amplitude.

Thus, the feedback information may be transferred either by a separate communication system including receivers and transmitters or may be integrated in the energy system. In accordance, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil.

The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off In implementing this system in the arrangement of FIG. 682, the switch 1026 is either separate and controlled by the internal control unit 102, or integrated in the internal control unit 102. It should be understood that the switch 1026 should be interpreted in its broadest embodiment. This means a transistor, MCU, MCPU, ASIC FPGA or a DA converter or any other electronic component or circuit that may switch the power on and off.

To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 1027 to the external signal receiver 200c. Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized.

This coupling factor information may also be used as a feedback during energy transfer. In such a case, the energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor.

With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 1026 is interconnected between the external energy source 200a and an operation device, such as an electric motor 1007 operating the apparatus 10. An external control unit 200b controls the operation of the external switch 1026 to effect proper operation of the apparatus 10.

FIG. 684 illustrates different embodiments for how received energy can be supplied to and used by the apparatus 10. Similar to the example of FIG. 682, an internal energy receiver 1002 receives wireless energy E from an external energy source 200a which is controlled by a transmission control unit 200b. The internal energy receiver 1002 may comprise a constant voltage circuit, indicated as a dashed box “constant V” in the figure, for supplying energy at constant voltage to the apparatus 10. The internal energy receiver 1002 may further comprise a constant current circuit, indicated as a dashed box “constant C” in the figure, for supplying energy at constant current to the apparatus 10.

The apparatus 10 comprises an energy consuming part 10a, which may be a motor, pump, restriction device, or any other medical appliance that requires energy for its electrical operation. The apparatus 10 may further comprise an energy storage device 10b for storing energy supplied from the internal energy receiver 1002. Thus, the supplied energy may be directly consumed by the energy consuming part 10a, or stored by the energy storage device 10b, or the supplied energy may be partly consumed and partly stored. The apparatus 10 may further comprise an energy stabilizing unit 10c for stabilizing the energy supplied from the internal energy receiver 1002. Thus, the energy may be supplied in a fluctuating manner such that it may be necessary to stabilize the energy before consumed or stored.

The energy supplied from the internal energy receiver 1002 may further be accumulated and/or stabilized by a separate energy stabilizing unit 1028 located outside the apparatus 10, before being consumed and/or stored by the apparatus 10. Alternatively, the energy stabilizing unit 1028 may be integrated in the internal energy receiver 1002. In either case, the energy stabilizing unit 1028 may comprise a constant voltage circuit and/or a constant current circuit.

It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope.

FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window.

The schematic FIG. 685 shows a circuit implementation for a system that transfers energy to the implanted energy components of the apparatus from outside of the patient's body using inductive energy transfer. An inductive energy transfer system typically uses an external transmitting coil and an internal receiving coil. The receiving coil, L1, is included in the schematic FIG. 668; the transmitting parts of the system are excluded.

The implementation of the general concept of energy balance and the way the information is transmitted to the external energy transmitter can of course be implemented in numerous different ways.

The schematic FIG. 685 and the above described method of evaluating and transmitting the information should only be regarded as examples of how to implement the control system.

### Circuit Details

In FIG. 685 the symbols Y1, Y2, Y3 and so on symbolize test points within the circuit. The components in the diagram and their respective values are values that work in this particular implementation which of course is only one of an infinite number of possible design solutions.

Energy to power the circuit is received by the energy receiving coil L1. Energy to implanted components is transmitted in this particular case at a frequency of 25 kHz. The energy balance output signal is present at test point Y1.

Those skilled in the art will realize that the above various embodiments of the system could be combined in many different ways. For example, the electric switch 1006 of FIG. 668 could be incorporated in any of the embodiments of FIGS. 671-677, the hydraulic valve shifting device 1014 of

FIG. 671 could be incorporated in the embodiment of FIG. 670, and the gear box 1024 could be incorporated in the embodiment of FIG. 669. Please observe that the switch simply could mean any electronic circuit or component.

The embodiments described in connection with FIGS. 682, 684 and 685 identify a method and a system for controlling transmission of wireless energy to implanted energy consuming components of an electrically operable apparatus. Such a method and system will be defined in general terms in the following.

A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance.

The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied.

When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

FIGS. 686-689 show in more detail block diagrams of four different ways of hydraulically or pneumatically powering an implanted apparatus.

FIG. 686 shows a system as described above with. The system comprises an implanted apparatus and further a separate regulation reservoir 1013, a one way pump 1009 and an alternate valve 1014.

FIG. 687 shows the apparatus 10 and a fluid reservoir 1013. By moving the wall of the regulation reservoir or changing the size of the same in any other different way, the adjustment of the apparatus may be performed without any valve, just free passage of fluid any time by moving the reservoir wall.

FIG. 688 shows the apparatus 10, a two way pump 1009 and the regulation reservoir 1013.

FIG. 689 shows a block diagram of a reversed servo system with a first closed system controlling a second closed system. The servo system comprises a regulation reservoir 1013 and a servo reservoir 1050. The servo reservoir 1050 mechanically controls an implanted apparatus 10 via a mechanical interconnection 1054. The apparatus has an expandable/contactable cavity. This cavity is preferably expanded or contracted by supplying hydraulic fluid from the larger adjustable reservoir 1052 in fluid connection with the apparatus 10. Alternatively, the cavity contains compressible gas, which can be compressed and expanded under the control of the servo reservoir 1050.

The servo reservoir 1050 can also be part of the apparatus itself.

In one embodiment, the regulation reservoir is placed subcutaneous under the patient's skin and is operated by pushing the outer surface thereof by means of a finger. This system is illustrated in FIGS. 690a-c. In FIG. 690a, a flexible subcutaneous regulation reservoir 1013 is shown connected to a bulge shaped servo reservoir 1050 by means of a conduit 1011. This bellow shaped servo reservoir 1050 is comprised in a flexible apparatus 10. In the state shown in FIG. 690a, the servo reservoir 1050 contains a minimum of fluid and most fluid is found in the regulation reservoir 1013. Due to the mechanical interconnection between the servo reservoir 1050 and the apparatus 10, the outer shape of the apparatus is contracted, i.e., it occupies less than its maximum volume. This maximum volume is shown with dashed lines in the figure.

FIG. 690b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 10 so that it occupies its maximum volume, thereby stretching the stomach wall (not shown), which it contacts.

The regulation reservoir 1013 is preferably provided with means 1013a for keeping its shape after compression. This means, which is schematically shown in the figure, will thus keep the apparatus in a stretched position also when the user releases the regulation reservoir. In this way, the regulation reservoir essentially operates as an on/off switch for the system.

An alternative embodiment of hydraulic or pneumatic operation will now be described with reference to FIGS. 691 and 692a-c. The block diagram shown in FIG. 691 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 10 having an expandable/contactable cavity is in turn controlled by the larger adjustable reservoir 1052 by supply of hydraulic fluid from the larger adjustable reservoir 1052 in fluid connection with the apparatus 10.

An example of this embodiment will now be described with reference to FIG. 692a-c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 692692a, the servo reservoir 1050 contains a minimum of fluid and most fluid is found in the regulation reservoir 1013.

The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 10. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 10. In other words, in this reversed servo, a small volume in the regulation reservoir is compressed with a higher force and this creates a movement of a larger total area with less force per area unit.

Like in the previous embodiment described above with reference to FIGS. 690a-c, the regulation reservoir 1013 is preferably provided with means 1013a for keeping its shape after compression. This means, which is schematically shown in the figure, will thus keep the apparatus 10 in a stretched position also when the user releases the regulation reservoir. In this way, the regulation reservoir essentially operates as an on/off switch for the system.

FIG. 374 shows an embodiment of the implantable device, wherein the implantable device comprises an eccentrically rotating member 891, being a driving member, being a part of an operation device having a rotating center 803. The operation device further comprises an embodiment of a magnetic motor, such as the magnetic motor described with reference to FIGS. 319 and 320 comprising coils 804 and magnets in magnetic connection with said coils 804. The coils 804 are placed on a first plate 812 which is in connection with a second plate 891, comprising the magnets. In the embodiment shown in FIG. 374, the second plate 891 comprises the eccentrically rotating member 891. The first 812 and second 891 plates are adapted to be rotationally displaceable in relation to each other, and a force is created by successive energizing of the coils 804 in magnetic connection with the magnets, which creates a rotational movement of the first plate 812 in relation to the second plate 891 which in turn affects the eccentrically rotating member 891. Further, according to the embodiment of FIG. 374, the first 812 and second 891 plates are adapted to be in contact with each other, in use, in a contacting surface which according to this embodiment comprises ceramic material for resisting wear.

The operation device is placed in a sealed chamber confined by the piston 801 and the sleeve 802. The piston 801 and sleeve 802 is according to this embodiment adapted to be in contact with each other and to create a seal in a contact point 807. The contact point 807 could comprise a ceramic material resistant to wear, which prolongs the life of the implantable device. According to the embodiment of FIG. 374, the eccentrically rotating member 891 is adapted to create movement of the piston 808 in a first direction, the movement in the opposite direction is created by spring members 805 which are loaded when the eccentrically rotating member 891 presses the piston 808 in the first direction. The piston 808 could be adapted to be in direct contact with the heart, or to affect an arm or heart contacting organ, which in turn is in contact with the heart.

FIG. 375 shows another embodiment of the implantable device, comprising a piston placed in a sleeve 802. The piston and the sleeve together confine a sealed space adapted to 806 receive a high pressured hydraulic fluid from an inlet 809. The high pressured hydraulic fluid is adapted to push the piston 801 in a first direction, whereas the vacuum created when the hydraulic fluid is sucked from the sealed space 806 through the outlet 810. The piston 801 is in contact with the sleeve 802 in a contact point 807, here being an area 807 between the sleeve 802 and the piston 801. The contacting area 807 could be made from a ceramic material and thereby adapted to better resist the wear that is created by the implantable device having to operate at the speed of the heart. The hydraulic fluid could for example be pressurized using a hydraulic pump. According to some embodiments the system is a pneumatic system in which case the implantable device is powered by a gas compressed by a pneumatic pump. In yet other embodiments (not shown) the piston 801 is adapted to be moved in the opposite direction by means of spring members 805, much like the embodiment of FIG. 374, this could be needed if the piston 801 and sleeve 802 are very tightly fitted for sealing against a very high pressure since the force exerted by vacuum is limited.

FIG. 376 shows a lateral view of a human patient in section where an implantable device for assisting the heart function is implanted. The heart H is placed in the pericardium P which is a heart covering sac in which the heart H is placed. The pericardium P rests on, and is fixated to the thoracic diaphragm D separating the thorax from the abdomen. The implantable device comprises a connecting arm 244 connecting a heart contacting organ 2 to a plate 242 fixated to the sternum 250 of the patient.

According to other embodiments the plate 242 or the fixation arm 244 could be fixated to at least one rib of the patient, or at least one vertebra. According to the embodiment of FIG. 376 the heart help device is a device adapted to compress the heart by exerting a force on the external part of the heart H, however in other embodiments the heart help device could be an artificial heart, or a LVAD device, fixated to a part of the human body comprising bone in the same way.

The heart rests on the superior surface of the thoracic diaphragm D. The pericardium P is a triple-layered sac that encloses the heart H. The outer layer being the fibrous pericardium adheres to the thoracic diaphragm D inferiorly and superiorly it is fused to the roots of the great vessels that leave and enter the heart H.

By creating the opening and placing a diaphragm contacting part 501, which according to some embodiments is a grommet, in the area of the thoracic diaphragm D in which the heart H rests it is possible to gain access to the pericardium P without actually entering the thoracic cavity outside of the pericardium P. The pressure in the thoracic cavity is somewhat different from the pressure in the abdominal cavity, which among other things makes it more advantageous to be able to connect a heart pump device engaging the heart H to an operating device placed in the abdominal cavity without entering the thoracic cavity outside of the pericardium P.

FIG. 377 shows a lateral view of a human patient in section where an implantable device for assisting the heart function is implanted. A connecting arm is fixated to a plate 241 which is fixated to a vertebra of the vertebral column using a screw 243, however alternative means of fastening is equivalently conceivable, such as pop rivets, adhesive or a fixating wire. The connecting arm is in turn fixating an operating device 57, adapted to operate the heart help device. From the operating device another portion of the connecting member 244 being a force transferring member 502 extends forward and upward in the figure. The force transferring member 502 is adapted to transfer force from the operating device 57 to the heart contacting organ 2 placed in connection with the heart. The force transfers force through a diaphragm contacting part 501, in this case being a grommet 501 placed in contact with the thoracic diaphragm and thereby assisting in the maintaining of an opening from the abdominal side of the thoracic diaphragm D to the thoracic side of the thoracic diaphragm D. In other embodiments the diaphragm contacting part is excluded and the force transferring member 502 (or diaphragm passing part) thereby transfers force through the thoracic diaphragm D, passing an opening in the thoracic diaphragm D without passing through a diaphragm contacting part

The operation device 57 could be an operation device adapted to create a mechanical force, a hydraulic force, a pneumatic force which is then transferred by the force transferring member 502. In other embodiments an energy supply such as a battery is placed in the abdomen and fixated to a part of the human body comprising bone. The electric energy is then transferred to through an electrical lead passing through the thoracic diaphragm D through the diaphragm contacting part 501 assisting in the maintaining of an opening in the thoracic diaphragm D. In other embodiments the electric energy is transferred through an opening in the thoracic diaphragm D through an opening in the thoracic diaphragm D without passing a diaphragm contacting part.

FIG. 378 shows a lateral view of a human patient in section where an implantable device for assisting the heart function is implanted. A connecting member 244 connects an operating device 57 to a rib 251 of the patient through a fixation plate 242 being fixated to said rib 251. The operating device 57 is in turn adapted to operate a force transferring member 502 placed between said operating device 57 and a heart contacting organ 2 adapted to be in contact with the heart H. The force transferring member is adapted to transfer force through a diaphragm contacting part 501 placed in the thoracic diaphragm and assisting in maintaining an opening in the thoracic diaphragm D and the pericardium P. This is further explained with reference to FIG. 377. The fixation plate 242 is here placed on the outside of the rib 251, however it is equally conceivable that the fixation plate 242 is placed on the inside. The fixation plate could for example be fixated to the rib 251 using screws which could be adapted to fixated the plate to the outer cortex of the rib 242, the inner cortex of the rib 251, both the inner and outer cortex of the rib 251, or in a through going embodiment wherein the screw thus clamps the rib 251 for example through a nut and bolt arrangement, or a second plate with threads placed on the inner or outer side of the rib 251.

FIG. 379 shows a lateral view of a human patient in section where an implantable device for assisting the heart function is implanted. In the embodiment of FIG. 379 a fixation plate 242 is fixated to the inside of the sternum 250. A connecting arm 244 is fixated to the connecting arm 244 and penetrates the thoracic diaphragm through a first diaphragm contacting part 501b. The connecting arm 244 in turn fixates an operating device 57 which operates a force transferring member 502 which in turn transfers force through the thoracic diaphragm D through a second diaphragm contacting part 501 to the heart help device comprising a heart contacting organ 2 adapted to be in contact with the heart H of the patient. The second heart contacting part 501 assists in the maintaining of an opening in the thoracic diaphragm D and the pericardium P. This is further explained with reference to FIG. 377, and the diaphragm contacting parts 501, 501b and force transferring member 502 is further described with reference to FIGS. 395-401.

FIG. 380 shows a lateral view of a human patient in section where an implantable device for assisting the heart function is implanted. In the embodiment of FIG. 380 a fixation plate 242 is fixated to the outside or anterior side of the sternum 250. A connecting arm 244 then passes along the sternum and in to the abdomen of the patient and is bent to extend in to the abdomen to a section of the thoracic diaphragm in which the pericardium P rests and is fixated to the thoracic diaphragm D. From the operating device 57 a force transferring member penetrates the thoracic diaphragm D through a diaphragm contacting part 501. The heart contacting organ 2 in contact with the heart 2 is a part of a heart help device adapted to assist the pump function of the heart by exerting a force on the external part of the heart. This embodiment enables a fixation of the operating device 57 and the heart help device in the abdomen without having to enter the thorax outside of the pericardium P. This makes it possible to separate the thorax from the abdomen which, among other aspects, is advantageous since there is a difference in pressure between the thorax and the abdomen.

FIG. 381 shows a surgical or laparoscopic method of creating and maintaining an opening in the thoracic diaphragm D of a patient. The method comprises the steps of: creating an incision 503 in the thoracic diaphragm D and thereby creating an opening 503 in the thoracic diaphragm D, placing a diaphragm contacting part 501 in contact with the thoracic diaphragm D, thereby maintaining the opening 501 created in the thoracic diaphragm D. According to the embodiment of FIG. 381 the opening 503 in the thoracic diaphragm D is made in the section of the thoracic diaphragm in which the pericardium rests and is fixated, the opening continues into the pericardium P of the patient, which create an opening reaching from the abdomen and into the pericardium enabling an element to be placed in contact with the heart H through the said opening 503. FIG. 381 further shows a section of a heart help device comprising a heart contacting organ 2, a connection arm 244, a fixation plate 242 and a screw 243 for fixation of the fixation plate 242. The connection arm is bent such that said connecting arm is adapted to fixate a heart help device to a part of the human body comprising bone through the diaphragm contacting part 501 maintaining an opening in the thoracic diaphragm D.

FIG. 382 shows a lateral view of a patient showing the heart H being placed in the pericardium P in the thorax resting on and being fixated to a section of the thoracic diaphragm D. FIG. 382 shows a illustrates a method of placing a heart help device through an incision in the thorax 506. The heart help device comprising a fixation plate 242, a connecting arm 244 and a heart contacting organ 2. The operation methods of FIGS. 381 and 382 could be performed as surgical methods or laparoscopic methods where the steps of the methods are performed through trocars placed in the thorax and abdomen, respectively.

FIG. 383 shows a close-up of part of the thoracic diaphragm D and the pericardium P in the section of the thoracic diaphragm D in which the pericardium P rests and is fixated. The diaphragm contacting part 501 is assisting in the maintaining of an opening in the thoracic diaphragm D and the pericardium P. The diaphragm contacting part 501 is a grommet like structure with protrusions 507 extending from the part of the diaphragm contacting part 501 defining the opening from the abdominal side of the thoracic diaphragm D to the thoracic side of the thoracic diaphragm D. The protrusions 507 clamps the edges of the opening in the thoracic diaphragm D and the pericardium and thereby assists in the fixation of the diaphragm contacting part to the thoracic diaphragm D and the pericardium P.

FIG. 384 shows an embodiment of a heart help device adapted to assist the pump function of the heart by exert force on the outside of the heart H. The heart H is placed in the pericardium P which rests and is fixated to the thoracic diaphragm D at a section of the thoracic diaphragm. FIG. 384 shows an embodiment where an operation device 57 is placed in the abdomen of a patient. A force transferring member 502 comprises a first and second portion. The first portion is connected to an operation device 57 placed in a sealing operation device container 518 adapted to protect the operation device from the environment of the abdomen. The second portion of the force transferring member 502 is connected to a force entering section 517 of the heart help device placed in the pericardium P. The force entering section transfers the force supplied by the force transferring member 502 to two arms 516 connected to two force transferring members 502a and 502b at a pivotable joint 515. The heart contacting organs 502a,b are adapted to be in contact with the heart H on the anterior and posterior side of the heart H for exerting force on the heart to assist the pump function thereof.

The force transferring part is adapted to transfer force through the thoracic diaphragm at a section of the thoracic diaphragm D in which the pericardium P rests and is fixated to the thoracic diaphragm D. An opening in the thoracic diaphragm D and the pericardium is maintained be a diaphragm contacting part 501 adapted to be in connection and fixated to the pericardium P and/or the thoracic diaphragm D.

The operating device shown in FIG. 384 is a magnetic operating device further disclosed with reference to FIGS. 319 and H8, however it is equally conceivable that the operating device is an electrical motor, a servo motor, a hydraulic motor or a pneumatic motor. The operating device could be adapted to create a rotational mechanical force and/or a translational mechanical force and/or an eccentrically rotating mechanical force.

FIG. 385 shows an embodiment of an implantable heart help device comprising the elements of the embodiment shown in FIG. 384. The embodiment of FIG. 385 further comprises a fibrotic tissue movement structure 560 being a bellows shaped elastic member with protrusions 561 and recesses 562 for enabling movement of the force transferring member even after fibrotic tissue has begun to grow on the fibrotic tissue movement structure 560 after the implantable device has been implanted in a patient for some time. The fibrotic tissue movement structure 560 is fixated to the sealing operation device container 518 placed in the abdomen of the patient, and to the diaphragm contacting part assisting in the maintaining of an opening in the thoracic diaphragm D. The force transferring part placed between the heart help device and the operation device container 518 placed in the abdomen comprises a first part 563 in connection with the operating device 57 and a second part 564 in connection with the heart help device. The first 563 and second 564 part constitutes a respiration movement compensator for compensating for the movements in the body created by the respiration of the patient. The respiration movement compensator is extend/compressible through a telescopic functionality. A guide pin 565 is fixated to the first part 563 and placed in a groove in the second part and the respiration movement compensator thereby enabled transfer of torque/rotational force while maintaining the ability to extend/compress for compensating for the movements in the body created by the respiration of the patient. FIG. 385 further shows a fixation member comprising a connecting arm 244 and a fixation plate.

The fixation member is adapted for fixating the implantable device to the outside of the sternum or at least one rib, however, embodiments where the fixation members is adapted to enable fixation of the implantable heart help device to the outside of the sternum or at least one rib is equally conceivable. To enable the respiration movement compensation to function the arms 516a,b are pivotably arranged to the diaphragm contacting part 501 and movable in relation to the operation device container 518.

FIG. 385 further shows a pericardial drainage device for draining a fluid from the pericardium P of a patient. The drainage device comprises a conduit comprising a first 980 and second 981 section. At portion of the first section 980 is adapted to receive a fluid inside of the pericardium P. The second section 981 of the conduit is adapted to be positioned outside of the pericardium P of the patient and enable the exhaust of the fluid received from the pericardium P through at least a portion of the second section 981.

The pericardial drainage of the embodiment of FIG. 385 is adapted move a fluid from the pericardium of the patient to the abdomen of the patient, however in other embodiments it is equally conceivable that the drainage device is adapted to move fluid from the pericardium to any other location in the body. The second section 981 could be connected to an implantable container 983 for collecting the drained fluid, or an exhaust member for exhausting the fluid into the abdomen of the patient.

FIG. 386 shows an alternative embodiment of the respiration movement compensator disclosed with reference to FIG. 385. This alternative embodiment enables movements around a spherically shaped connecting part of the first part 563. The connecting part comprising splines 565 adapted to be placed in corresponding splines in the second part 564 for enabling the transfer of torque while maintain the ability to move in multiple directions. FIG. 387 shows the respiratory movement compensator when the first part 563 is tilted in the second part 564.

FIG. 388 shows the implantable heart help comprising the elements of the heart help device disclosed with reference to FIG. 384. The heart contacting organs 502a,b of FIG. 388 further comprises hydraulic or pneumatic cushions 171 adapted to exert force on the heart H. The hydraulic or pneumatic cushions 171 could change to alter the area of the heart H to which force is exerted. The cushions comprise chambers having a volume and the size of that volume is adapted to be changeable individually, for each cushion to influence the force exerted on the heart H after the implantable heart help device has been implanted in the patient. The hydraulic or pneumatic cushions have volumes adapted to be changed using an implantable hydraulic or pneumatic system 519, according to this embodiment adapted to be placed in the abdomen of the patient. The hydraulic or pneumatic system comprises multiple conduits 514, which according to this embodiment separates into two section 514a,b for enabling movement of the cushions 171 of the first and second heart contacting organ 502a,b. The hydraulic or pneumatic conduits 514 is according to this embodiment adapted to transfer force through an opening in the thoracic diaphragm D adapted to be maintained by a diaphragm contacting part 501. In the embodiment of FIG. 388 the diaphragm contacting part is thus adapted to allow both a mechanical force transferring member 502 and a hydraulic pneumatic force transferring member to pass through the diaphragm contacting part 501. In other embodiments (not shown) the implantable heart help device further comprises an electric system at least partially adapted to be placed in the abdomen of the patient and comprising an electric lead adapted to transfer electric energy, an electric control signal or sensor input to or from the part of the implantable heart help device placed in the thorax of the patient. The heart help device according to any of the embodiments herein could comprise one or more sensors providing input. This could in any of the embodiments herein for example be a signal relating to the heart rhythm, the blood pressure, the blood flow, electric activity of the heart, temperature, time or variable relating to the content of the blood, such as saturation, sodium, erythrocytes, leukocytes and/or thrombocytes. The heart help device according to any of the embodiment herein could further be equipped with at least one electrode supplying an electric signal for controlling the heart rhythm, such as a pacemaker signal. The energizing system or control unit for handling the sensor signals could be adapted to be placed in the abdomen of the patient.

FIG. 389 shows the implantable heart help device in an embodiment where the heart help device comprises a hydraulic system for controlling a plurality of hydraulic cushions 171a-e. The hydraulic system comprises an implantable injection port unit 527. The injection port unit 527 comprising a plurality of chambers 524a-e each comprising wall sections being penetrable self-sealing membranes 528a-d adapted to be penetrated by a needle 529 attached to an injecting member 530 for injecting a fluid into the chambers 524a-e. The needle is inserted through an insertion guide 526 fixated to human tissue 525 for example by subcutaneous implantation. The needle is then inserted through one or more of the wall sections 528a-d for injecting a fluid into a specific chamber 524a-e and thereby affect a specific cushion 171a-e and by the connection through the conduits 514a-e. In the embodiment shown in FIG. 389 the plurality of conduits is bundled into a conduit bundle 531.

The location on the needle 529, i.e. in which chamber 524a-e the fluid is injected could be controlled by a system of sensors that by for example induction feels the presence of the needle 529 in a specific chamber 524a-e. The system of sensors could be adapted to wirelessly transmit the signals to the physician injecting the fluid into the system. It is furthermore conceivable that the system comprises sensors sensing the amount of hydraulic fluid injected to specific chambers 524a-e and thereby how much each cushion 171a-e has been affected.

FIG. 390 shows an alternative design of the injection port unit as described with reference to FIG. 389. The injection port unit here has the plurality of chambers 524a-e placed next to each other and thereby the needle does not have to penetrate several wall portions to reach a specific chamber 524a-e.

FIG. 391 shows an embodiment of a hydraulic system for supplying force to an implantable heart help device. The hydraulic system comprises a cylinder 904 in which a piston 905 is placed such that a first and second chamber 906a,b exists on the two sides of the piston 905. The piston 905 is adapted to move in said cylinder 904 in response to the chambers 906a,b being pressurized using a hydraulic or pneumatic fluid F. The system further comprises a first and second conduit 907a,b for transferring the hydraulic or pneumatic fluid F to the two chambers 906a,b.

Two chambers 909 and 910a comprises the hydraulic or pneumatic fluid F. The first chamber 909 is adapted to be a high pressure chamber and adapted to hold a fluid F having a high pressure. The second chamber 910a is adapted to be a low pressure chamber. The pressure is maintained by a pressurized gas 911 being confined behind a membrane of the chamber and thereby exerting a pressure on the fluid in the chamber 909. The fluid is transported to a valve 908 that has two states. In the first state of the valve the valve guides the fluid from the first high pressure chamber to the second cylinder chamber 906b pressing the cylinder 905 upwards in the FIG. In this state the valve also enables the fluid from the first cylinder chamber 906a to be pressed into the conduit 907a and through the valve and into the low pressure chamber 910a. The fluid is then pumped to the high pressure chamber 909 using a pump 915 placed between a first 913a and second 912a part of a conduit. A check valve is further placed on the conduit for enabling the pressure in the high pressure chamber 909 to remain high even when the pump 915 is turned off. At a second state of the valve 908 the fluid is guided from the high pressure chamber 909 through the conduit 907a and into the first cylinder chamber 906a, which thereby pushes the cylinder downwards in the figure. The second cylinder chamber is thereby emptied in a procedure analogue to what was described for the first cylinder chamber 906a and the fluid is passed to the low pressure chamber 910a. The cylinder 905 is connected to a rod 903 transferring the force to a heart contacting organ 902, directly, as disclosed in FIG. 391, or via an intermediary part. The system further comprises an injection port 917 for refilling or calibrating the system. The injection port 917 is implanted subcutaneously and fixated to a tissue of the body 918 and connected to the low pressure chamber 910a by a conduit 916.

By the function of the system disclosed with reference to FIG. 391 the system can move the cylinder 905 and thereby the heart contacting organ 902 using a pressurized fluid F in two directions, which eliminated the limitation in force that operation by vacuum places on a system.

FIG. 392 shows a hydraulic system with similar functionality as the system of FIG. 389. A high pressure chamber 909, comprising a gas pressure 911, presses a fluid F, which is in contact with a valve through a conduit 921. The valve 920 is adapted to direct the fluid to a plurality of conduits 919 in connection with a plurality of pistons 922 in connection with a heart contacting organ, for changing the area of the heart in which force is exerted, the pistons being placed on a plate 923.

FIG. 393 shows a closed system with similar functionality as the system of FIG. 392. A first cylinder system 930 with a first cylinder 932 and a first piston 931 is adapted to press a fluid through a first conduit 933 to a valve 934. The valve is adapted to be operable to select conduits to direct the force coming from the fluid pressurized by the first cylinder system 930. The conduits are connected to several cylinder systems 936 adapted to receive the force from the first cylinder system 930 and/or transmit force back to the first cylinder system 930. The first cylinder system 930 could be adapted to be connected to an operating device, as disclosed with reference to FIG. 349 for powering the system. By the function described with reference to FIG. 393 a fully implantable system is disclosed for transferring force from one location to several others using a selection valve 934.

FIG. 394 discloses an implantable heart help device similar to the embodiment disclosed with reference to FIG. 384 with the big difference that the heart help device is operated totally hydraulic by a hydraulic system 519b placed in the abdomen and in a connection with a conduit 514 adapted to transfer force through an opening in the thoracic diaphragm though a diaphragm contacting part 501 adapted to assist in the maintaining of the opening in the thoracic diaphragm D. The conduit transfers force to a force entering section 517 adapted to transform the hydraulic force to mechanical force for exerting force on the heart H by the arms 516 pivotally connected at a joint 515 to the heart contacting organs 502a,b.

The hydraulic or pneumatic system 519b could comprise a hydraulic or pneumatic pump creating the force. The system could be powered or controlled non-invasively from outside the body.

FIG. 395a-d shows an embodiment of the diaphragm contacting part disclosed in several embodiments throughout the application. The diaphragm contacting part of FIG. 395a is a diaphragm contacting part adapted to be opened to enable the insertion of force transferring members or diaphragm passing parts. The diaphragm contacting part comprises an outer section 509 which is adapted to engage the edges of an opening created in the thoracic diaphragm. The edges 507 of the thoracic diaphragm could clamp the thoracic diaphragm and thereby assist in the fixation of the diaphragm contacting part to the thoracic diaphragm and/or to the pericardium. The diaphragm contacting part could be closed by means of protrusions 510 in one part of the opening and recesses 511 in the other part of the opening.

The protrusions and recesses match and thereby supply a mechanical fixation of the diaphragm contacting part. FIG. 395b shows the diaphragm contacting part possible to open in its closed state. The inner surface 508 of the diaphragm contacting part is smooth not to injure any force transferring member or diaphragm passing part. The inner surface 508 could be made of a highly durable material such as a ceramic material for better resisting the wear that direct contact with a force transferring part creates.

FIG. 395c shows an embodiment of the diaphragm contacting part in which the diaphragm contacting part is a solid ring without the functionality of being able to be opened. The diaphragm contacting part is similar to a grommet and has basically the same functionality. FIG. 395d shows the solid ring in section.

FIG. 396 shows the diaphragm contacting part in an embodiment when a force transferring member 502 has been placed in the diaphragm contacting part to enable the transfer of force from the abdominal said of the thoracic diaphragm to the thoracic side of the thoracic diaphragm.

FIG. 397 shows diaphragm contacting part in an embodiment where two force transferring members 502a,b are placed in the diaphragm contacting part, for transferring mechanical force from the abdominal side of the thoracic diaphragm to the thoracic side of the thoracic diaphragm. According to the embodiment shown in FIG. 397 the force transferring member 502b is adapted to transfer a translating or reciprocating force, whereas the force transferring member 502a is adapted to transfer a rotating force.

FIG. 398 shows a force transferring member 502 placed in the diaphragm contacting part, in an embodiment where the force transferring member 502 is adapted to seal against the diaphragm contacting part 501 and thereby seal the abdominal cavity from the thoracic cavity, which is beneficial since there could be difference in pressure between the abdominal cavity and the thoracic cavity. The seal is created in a contacting point 513. The surfaces of the contacting points 513 could be made of a highly durable material for resisting the wear, such as a ceramic material, for resisting the wear created by the constant contact between the diaphragm contacting part 501 and the force transferring member 502.

FIG. 399 shows the diaphragm contacting part in an embodiment in which a conduit 514 is placed in the diaphragm contacting part for enabling the transfer of hydraulic force from the abdominal side of the thoracic diaphragm to the thoracic side of the thoracic diaphragm.

FIG. 400 shows the diaphragm contacting part in an embodiment where one force transferring member 502 for transferring mechanical force, and one force transferring member 514 for transferring hydraulic force is placed in the diaphragm contacting part.

FIG. 401 shows an embodiment in which the force transferring part 502 is placed in the thoracic diaphragm D without the use of a diaphragm contacting part 501. The force transferring part is thus adapted to assist in the maintaining of an opening in the thoracic diaphragm D. The force transferring member 502 could be adapted to be in contact with the thoracic diaphragm when the force transferring member is placed in the opening in the thoracic diaphragm and thereby transferring force from the abdominal cavity to the thoracic cavity while sliding against the thoracic diaphragm.

FIG. 402a shows an embodiment of a heart help device adapted to exert a force on the heart. The heart help device comprises a fixation plate 242 for enabling fixation of the device to a part of the human body comprising bone though screws being placed in the fixation holes 610 in the plate 242. A magnetic operating device 600 is mounted onto the plate for operating the heart contacting organs 602a,b adapted to exert a force on the heart. According to some embodiments the heart contacting organs 602a,b are hydraulic or pneumatic cushions, the function thereof being described with reference to other figures herein. A first arm 616a connects the part comprising the operating device 600 to a hinged 604 second arm 616b which enables the movement of the second arm 616b in relation to the first arm 616a. A first heart contacting organ 602a is operably mounted to a plate 615 adapted to enable movement of the first heart contacting organ 602a for changing the location of the force exerted on the heart. The plate is operable by a gear connection 614; 613 between the plate 615 and a motor 612 adapted to operate the plate 615. The force exertion on the heart is performed by the operation device 600 being in connection with a driving member performing an eccentric rotating movement of a fixation point 609 to which a driving wire 621 is fixated and thereby pulling of the second hinged arm 616b, thereby creating the movement exerting force on the heart. The heart help device is by this construction periodically exerting force on the heart muscle following the heart contractions and adding force thereto.

FIG. 402b shows the implantable heart help device in a second view disclosing the movement functionality adapted to alter the position of the heart help device and the heart contacting organs, thereby altering the position of the force exerted on the heart, from a first area of the heart to a second area of the heart. The operating device comprises a first motor 605 adapted to affect a gear functionality 608 creating a translating movement of the heart pump device in relation to the fixation plate 242. The implantable device further comprises a unit 607 adapted to enabling a rotating movement of the heart pump device in relation to the fixation plate 242. For securing the position the operating device further comprises a locking member 606 for locking the heart help device in a specific position for exerting force on the heart. The unit 607 further comprises the operating device adapted to rotate the eccentrically rotating fixation point 609 pulling on the operation wire 621 creating the force exerted on the heart.

According to this embodiment the arms are spring loaded by a spring 603 in an outwards direction, which pulls the arms 616a,b apart after the operating wire 621 has pulled the arms 616a,b together. The entire system could be adapted to be controlled non-invasively from the outside, e.g. by means of a remote control. The system could then have sensor functionality for sending feedback on the location and operations of the device to outside the body, for example by means of wireless transfer. It is also conceivable that a scale 611 is made from radiologically dense material thus enable the scale to be read on a radiological image.

FIG. 403 shows the operating device in further detail. The operating device comprises a first part 640 having a first surface, and a second part 641 having a second surface, and a third part 642 having a third surface. The second part is displaceable in relation to the second and third part. The first, second and third surfaces are adapted to abut each other, at least partially. The first part exerts indirectly force on an external part of the heart by the connection with the drive wire 621. The first, second and third surfaces are substantially parallel. The second part comprises magnets 15 and the first and third parts comprise coils 14 and the displacement of the second part is created through successive energizing of the coils 14. The force from the displacement is transferred to the dive wire through a gear system 643, 644 in connection with the eccentric drive member comprising the eccentrically rotating fixation member 609 in which the drive wire 621 is fixated.

FIG. 404 shows the first part 640 comprising coils 14 when the second plate has been removed, however the FIG. also shows the magnets 15 from the second plate, even though the second plate has been removed.

FIG. 405 shows an embodiment of heart help device in which the heart help device comprises two heart contacting organs 702 which are adapted to exert a force on the anterior and posterior side of the heart, respectively. The heart contacting organs 702 are pivotally arranged in a joint 712. One surface of the heart contacting organs 702 are in contact with an eccentrically rotating driving member 711 operated by an operating device 710 by a connection with a first gear system 718, which transfers force from the operating device 710 to a force transferring member 720 to a second gear system 714 in close connection to the eccentrically rotating member 711. The eccentrically rotating member and/or the surface of the heart contacting organs contacting the eccentrically rotating driving member could be made of a durable material, such as a ceramic material, for resisting the wear created by the constant connection of the eccentrically rotating member 711 with the heart contacting organ. The pump device of the implantable heart help device is hinged to an arm 705 connected to a device 706 enabling the movement of the heart pump device along a fixation plate 708 comprising two fixation members 704 for fixating the fixation plate 708 to a part of the human body comprising bone. The entire system could be adapted to be controlled non-invasively from the outside, e.g. by means of a remote control. The system could then have sensor functionality for sending feedback on the location and operations of the device to outside the body, for example by means of wireless transfer.

FIG. 406a shows an embodiment of the heart help device similar to the device shown with reference to FIG. 405. However the device according to FIG. 323 is adapted to enter the pericardium from the abdomen in the area of the thoracic diaphragm D to which the pericardium P rests and is fixated. This method of placement enables the placement of the device without entering into the thorax of the patient, facilitating the procedure. The device is fixated to a part of the human body comprising bone through a fixation arm 742 which in turn supports an operation device 741 placed in the abdomen of the patient.

The operation device 741 transfers force through a force transferring member 740 connected to a linking part 710a to which two force transferring members 720 are attached. The device is adapted to travel through an opening in the thoracic diaphragm D being maintained by a diaphragm contacting part 501 fixated to the thoracic diaphragm and the pericardium.

FIG. 406b shows the device of FIG. 406b in its unfolded state with the operation device 741 fixated to the fixation plate 708 by means of a connecting arm 742 which according to this embodiment is operable by means of a position operation device 706 to alter the position of the heart help device in relation to the fixation plate 708. The features of other embodiments such as the respiratory movement compensator, the pericardial drain and the fibrotic tissue movement structure disclosed, with reference to FIG. 385 are of equal relevance and could be included in the embodiments of FIG. 406a,b.

FIG. 407 shows a flow-chart of an operation method which could comprise the steps of. 1) dissecting a part of the human body comprising bone and 2) fixating a fixating member to the bone, such that the fixation member is placed in contact with the connection arm. In one embodiment of this surgical procedure the method further comprises the steps of 3) creating an opening in the thoracic diaphragm and 4) inserting the connecting arm into the thorax through the opening in the thoracic diaphragm. This diaphragm approach enables a surgeon to place a heart help device in the pericardium of thorax without opening the thorax. The method could further comprise the step of placing an operation device in the abdomen of the patient, transferring force to through an opening in the thoracic diaphragm and into the thorax for operating a hart help device placed in thorax.

### I: Male Sexual Impotence Treatment Prosthesis

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a male sexual impotence treatment prosthesis, examples of such male sexual impotence treatment prosthesis will now be described.

FIG. 408 schematically shows a most simple embodiment of the male impotence prosthesis apparatus 100 having some parts implanted in a patient and other parts located outside the patient's body.

Thus, in FIG. 408 all parts placed to the right of the patient's skin SK are implanted and all parts placed to the left of the skin SK are located outside the patient's body.

The apparatus of FIG. 408 comprises an operable penile prosthesis 4 placed in the cavities of the corpora cavernosa of an impotent patient's penis. The implanted prosthesis 4 is capable of performing a reversible function, i.e. to erect the penis or to make the penis flaccid. An implanted energy transforming device 6 is adapted to supply energy consuming components of the penile prosthesis 4 with energy via a power supply line 12. An external energy transmission device 200 includes a wireless remote control transmitting a wireless signal, which is received by a signal receiver incorporated in the implanted energy transforming device 6. The implanted energy transforming device 6 transforms energy from the signal into electric energy which is supplied via the power supply line 12.

FIG. 409 shows an embodiment identical to that of FIG. 408, except that a reversing device in the form of an electric switch 14 operable by polarized energy also is implanted in the patient for reversing the penile prosthesis 4. The wireless remote control of the external energy transmission device 200 transmits a wireless signal that carries polarized energy and the implanted energy transforming device 6 transforms the wireless polarized energy into a polarized current for operating the switch 14. When the polarity of the current is shifted by the energy transforming device 6 the switch 14 reverses the function performed by the penile prosthesis 4.

FIG. 410 shows an embodiment identical to that of FIG. 408, except that an operation device in the form of a motor 15 for operating the penile prosthesis 4 also is implanted in the patient. The motor 15 is powered with energy from the energy transforming device 6, as the remote control of the external energy transmission device 200 transmits a wireless signal to the receiver of the energy transforming device 6.

FIG. 411 shows an embodiment identical to that of FIG. 408, except that an assembly 16 including a motor/pump unit 18 and a fluid reservoir 20 also is implanted in the patient. In this case the penile prosthesis 4 is hydraulically operated, i.e. hydraulic fluid is pumped by the motor/pump unit 18 from the reservoir 20 through a conduit 22 to the penile prosthesis 4 to erect the patients penis, and hydraulic fluid is pumped by the motor/pump unit 18 back from the penile prosthesis 4 to the reservoir 20 to make the penis flaccid. The implanted energy transforming device unit 6 transforms wireless energy into a current, for example a polarized current, for powering the motor/pump unit 18 via an electric power supply line 24.

FIG. 412 shows an embodiment comprising the external energy transmission device 200 with its wireless remote control, the penile prosthesis 4, in this case hydraulically operated, and the implanted energy transforming device 6, and further comprising an implanted hydraulic fluid reservoir 30, an implanted motor/pump unit 32 and an implanted reversing device in the form of a hydraulic valve shifting device 34. The motor of the motor/pump unit 32 is an electric motor. In response to a control signal from the wireless remote control of the external energy transmission device 200, the implanted energy transforming device 6 powers the motor/pump unit 32 with energy from the energy carried by the control signal, whereby the motor/pump unit 32 distributes hydraulic fluid between the reservoir 30 and the penile prosthesis 4. The remote control of the energy transmission device 200 controls the shifting device 34 to shift the hydraulic fluid flow direction between one direction in which the fluid is pumped by the motor/pump unit 32 from the reservoir 30 to the penile prosthesis 4 to erect the penis, and another opposite direction in which the fluid is pumped by the motor/pump unit 32 back from the penile prosthesis 4 to the reservoir 30 to make the penis flaccid.

FIG. 413 shows an embodiment identical to that of FIG. 408, except that a control unit 102 controlled by the wireless remote control of the external energy transmission device 200, an accumulator 38 and a capacitor 40 also are implanted in the patient. The control unit 102 stores electric energy received from the energy transforming device 6 in the accumulator 38, which supplies energy to the penile prosthesis 4. In response to a control signal from the wireless remote control of the energy transmission device 200, the control unit 6 either releases electric energy from the accumulator 38 and transforms the released energy via power lines 42 and 44, or directly transforms electric energy from the energy transforming device 6 via a power line 46, the capacitor 40, which stabilizes the electric current, a power line 48 and the power line 44, for the operation of the penile prosthesis 4.

In accordance with an alternative, the capacitor 40 in the embodiment of FIG. 413 may be omitted. In accordance with another alternative, the accumulator 38 in this embodiment may be omitted.

FIG. 414 shows an embodiment identical to that of FIG. 408, except that a battery 50 for supplying energy for the operation of the penile prosthesis 4 and an electric switch 52 for switching the operation of the penile prosthesis 4 also are implanted in the patient. The switch 52 is operated by the energy supplied by the energy transforming device 6 to switch from an off mode, in which the battery 50 is not in use, to an on mode, in which the battery 50 supplies energy for the operation of the penile prosthesis 4.

FIG. 415 shows an embodiment identical to that of FIG. 414, except that a control unit 102 controllable by the wireless remote control of the external energy transmission device 200 also is implanted in the patient. In this case, the switch 52 is operated by the energy supplied by the energy transforming device 6 to switch from an off mode, in which the wireless remote control is prevented from controlling the control unit 102102 and the battery is not in use, to a standby mode, in which the remote control is permitted to control the control unit 102 to release electric energy from the battery 50 for the operation of the penile prosthesis 4.

FIG. 416 shows an embodiment identical to that of FIG. 415, except that an accumulator 38 is substituted for the battery 50 and the implanted components are interconnected differently. In this case, the accumulator 38 stores energy from the energy transforming device 6. In response to a control signal from the wireless remote control of the external energy transmission device 200, the implanted control unit 102 controls the switch 52 to switch from an off mode, in which the accumulator 38 is not in use, to an on mode, in which the accumulator 38 supplies energy for the operation of the penile prosthesis 4.

FIG. 417 shows an embodiment identical to that of FIG. 416, except that a battery 50 also is implanted in the patient and the implanted components are interconnected differently. In response to a control signal from the wireless remote control of the external energy transmission device 200, the implanted control unit 102 controls the accumulator 38 to deliver energy for operating the switch 52 to switch from an off mode, in which the battery 50 is not in use, to an on mode, in which the battery 50 supplies electric energy for the operation of the penile prosthesis 4.

Alternatively, the switch 52 may be operated by energy supplied by the accumulator 38 to switch from an off mode, in which the wireless remote control is prevented from controlling the battery 50 to supply electric energy and is not in use, to a standby mode, in which the wireless remote control is permitted to control the battery 50 to supply electric energy for the operation of the penile prosthesis 4.

FIG. 418 shows an embodiment identical to that of FIG. 414, except that a motor 15, a mechanical reversing device in the form of a gear box 54 and a control unit 102 for controlling the gear box 54 also are implanted in the patient. The implanted control unit 102 controls the gear box 54 to reverse the function performed by the penile prosthesis 4 (mechanically operated).

FIG. 419 shows an embodiment identical to that of FIG. 417 except that the implanted components are interconnected differently. Thus, in this case the control unit 102 is powered by the battery 50 when the accumulator 38, suitably a capacitor, activates the switch 52 to switch to an on mode. When the switch 52 is in its on mode the control unit 102 is permitted to control the battery 50 to supply, or not supply, energy for the operation of the penile prosthesis 4.

FIG. 420 schematically shows conceivable combinations of implanted components of the apparatus for achieving various communication options. Basically, there are the implanted penile prosthesis 4, control unit 102 and motor/pump unit 18, and the external energy transmission device 200 including the external wireless remote control. As already described above the wireless remote control transmits a control signal which is received by the implanted control unit 102, which in turn controls the various implanted components of the apparatus.

A sensor 56 may be implanted in the patient for sensing a physical parameter of the patient, such as the pressure in the erected penis tissue. The implanted control unit 102, or alternatively the external wireless remote control of the energy transmission device 200, may control the penile prosthesis 4 in response to signals from the sensor 56. A transceiver may be combined with the sensor 56 for sending information on the sensed physical parameter to the external wireless remote control. The wireless remote control may comprise a signal transmitter or transceiver and the implanted control unit 102 may comprise a signal receiver or transceiver. Alternatively, the wireless remote control may comprise a signal receiver or transceiver and the implanted control unit 102 may comprise a signal transmitter or transceiver. The above transceivers, transmitters and receivers may be used for sending information or data related to the penile prosthesis 4 from inside the patient's body to the outside thereof.

Where the motor/pump unit 18 and battery 50 for powering the motor/pump unit 18 are implanted, the battery 50 may be equipped with a transceiver for sending information on the condition of the battery 50.

Those skilled in the art will realize that the above various embodiments according to FIGS. 408-420 could be combined in many different ways. For example, the polarized energy operated switch 14 could be incorporated in any of the embodiments of FIGS. 410, 413-419, the hydraulic shifting device 34 could be incorporated in the embodiment of FIG. 411, and the gear box 54 could be incorporated in the embodiment of FIG. 410.

FIG. 421 shows an energy transforming device in the form of an electrical junction element 58 for use in any of the above embodiments according to FIGS. 408-410. The element 58 is a flat p-n junction element comprising a p-type semiconductor layer 60 and an n-type semiconductor layer 62 sandwiched together. A light bulb 64 is electrically connected to opposite sides of the element 58 to illustrate how the generated current is obtained. The output of current from such a p-n junction element 58 is correlated to the temperature. See the formula below.

I=I0(exp(qV/kT)−1)
where
I is the external current flow,
is the reverse saturation current,
q is the fundamental electronic charge of 1.602×10-19 coulombs,
V is the applied voltage,
k is the Boltzmann constant, and
T is the absolute temperature.

Under large negative applied voltage (reverse bias), the exponential term becomes negligible compared to 1.0, and I is approximately −I0. I0 is strongly dependent on the temperature of the junction and hence on the intrinsic-carrier concentration. I0 is larger for materials with smaller bandgaps than for those with larger bandgaps. The rectifier action of the diode—that is, its restriction of current flow to only one direction—is in this particular embodiment the key to the operation of the p-n junction element 58.

An alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers such as but not limited to cadmium telluride, copper-indium-diselenide and silicon. It is also possible to use a multilayer structure with several layers of p and n-type materials to improve efficiency.

The electric energy generated by the p-n junction element 58 could be of the same type as generated by solar cells, in which the negative and positive fields create a direct current. Alternatively, the negative and positive semiconductor layers may change polarity following the transmitted waves, thereby generating an alternating current.

The p-n junction element 58 is designed to make it suited for implantation. Thus, all the external surfaces of the element 58 in contact with the human body are made of a biocompatible material. The p-n junction semiconductors are designed to operate optimally at a body temperature of 37 C because the current output, which should be more than 1 A, is significantly depending on temperature as shown above. Since both the skin and subcutis absorb energy, the relation between the sensitivity or working area of the element 58 and the intensity or strength of the wireless energy transmission is considered. The p-n junction element 58 preferably is designed flat and small. Alternatively, if the element 58 is made in larger sizes it should be flexible, in order to adapt to the patient's body movements. The volume of the element 58 should be kept less than 2000 cm³.

FIG. 422 generally illustrates how any of the above-described embodiments of the male impotence prosthesis apparatus may be implanted in a patient. Thus, a penile prosthesis 4 implanted in a patient engages the penile tissue and the prolongation thereof to provide flaccid or erected conditions of the patient's penis. An implanted operation device 68, such as an electric motor or a motor/pump assembly, operates the penile prosthesis 4 through a transmission member 70, such as a mechanical transmission cord or a fluid tube. An energy transforming device in the form of an element 6 having a positive region and a negative region, as described above in more detail, is placed underneath the skin of the patient.

Wireless energy carried by a signal transmitted by a wireless remote control of an external energy transmission device 200 at least partly penetrates the patient's skin and hits the element 6. The energy thus hitting the element 6 is transformed into energy of a different form that is suited for powering the operation device 68. For example, where the operation device 68 is an electric motor the element 6 comprises an electric p-n junction element that transforms the wireless energy into an electric current for powering the electric motor. Where the operation device 68 comprises a pump, the element 6 may transform the wireless energy into kinetic energy for powering the pump.

The transformed energy may be utilized for directly operating the penile prosthesis 4 or, where the penile prosthesis 4 is electrically operated, for storage in a capacitor and/or an accumulator for later or parallel use. Preferably (but not necessarily) the element 6 is controlled by a microprocessor. The wireless remote control of the external energy transmission device 200 is used to control the utilization of the transmitted energy and any function or command to/from the implanted penile prosthesis 4.

FIG. 423 shows the basic parts of a wireless remote control of the apparatus including an electric motor 128 for operating a restriction member, for example of the type illustrated in FIG. 422. In this case, the remote control is based on the transmission of electromagnetic wave signals, often of high frequencies in the order of 100 kHz-1 gHz, through the skin SK of the patient. In FIG. 422, all parts placed to the left of the skin SK are located outside the patient's body and all parts placed to the right of the skin SK are implanted. Any suitable remote control system may be used.

An external signal transmitting antenna 132 is to be positioned close to a signal receiving antenna 134 implanted close to the skin SK. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin, or in the scrotum and pelvic region of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134.

An external control unit 102 comprises a microprocessor, a high frequency electromagnetic wave signal generator and a power amplifier. The microprocessor of the control unit 102 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 102 is connected to the microprocessor thereof. The keypad is used to order the microprocessor to send digital signals to either contract or enlarge the penile prosthesis.

The microprocessor starts a command by applying a high frequency signal on the antenna 132. After a short time, when the signal has energized the implanted parts of the control system, commands are sent to contract or enlarge the penile prosthesis in predefined steps. The commands are sent as digital packets in the form illustrated below.

| | | | |
|---|---|---|---|
| Start pattern, | Command, | Count, | Checksum, |
| 8 bits | 8 bits | 8 bits | 8 bits |

The commands are sent continuously during a rather long time period (e.g. about 30 seconds or more). When a new contract or enlarge step is desired the Count byte is increased by one to allow the implanted control unit 138 to decode and understand that another step is demanded by the external control unit 102. If any part of the digital packet is erroneous, its content is simply ignored.

Through a line 140, an implanted energizer unit 126 draws energy from the high frequency electromagnetic wave signals received by the receiving antenna 134. The energizer unit 126 stores the energy in an energy storage device, such as a large capacitor, powers the control unit 138 and powers the electric motor 128 via a line 142.

The control unit 138 comprises a demodulator and a microprocessor. The demodulator demodulates digital signals sent from the external control unit 102. The microprocessor of the control unit 138 receives the digital packet, decodes it and, provided that the power supply of the energizer unit 126 has sufficient energy stored, sends a signal via a signal line 144 to the motor 128 to either contract or enlarge the penile prosthesis depending on the received command code.

Alternatively, the energy stored in the energy storage device of the energizer unit may only be used for powering a switch, and the energy for powering the motor 128 may be obtained from another implanted energy source of relatively high capacity, for example a battery. In this case the switch is adapted to connect said battery to the control unit 138 in an on mode when said switch is powered by the energy storage device and to keep the battery disconnected from the control unit in a standby mode when the switch is unpowered.

With reference to FIG. 423, the remote control schematically described above will now be described in accordance with a more detailed embodiment as in FIG. 424. The external control unit 1102 comprises a microprocessor 146, a signal generator 148 and a power amplifier 150 connected thereto.

The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148.

The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation.

A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178.

The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

The invention also comprises or consists of the foregoing structures and method steps, and is to be interpreted as broadly as allowed by the prior art.

### J: Affecting an Aneurysm on a Blood Vessel

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for affecting an aneurysm on a blood vessel, examples of such devices for affecting an aneurysm on a blood vessel will now be described

In FIG. 425 a general view of a human having a member 100, in particular a cuff 101, implanted for treating an aneurism is shown. In FIG. 425 the treated aneurism is located on the aorta in the abdomen close to the Y-bifurcation extending to the legs. The cuff 101 can be designed in various ways but is generally formed as an implantable member adapted to be placed in connection with a blood vessel having said vascular aneurysm, and adapted to exert a pressure on said aneurysm from the outside of said blood vessel. In particular the pressure exerted on the blood vessel is essentially uniform from all direction and adapted to hinder the blood vessel to expand in all directions thereby acting to prevent the blood vessel from bursting. The pressure can in accordance with one embodiment be essentially equal to or lower than the diastolic blood pressure of the treated patient. The cuff 101 can be made in any suitable material such as an elastic material adapted for implantation in a human or mammal body.

The cuff 101 can exercise the pressure in a number of different ways. In accordance with one embodiment of the present invention the pressure applied on the blood vessel can be mechanical and adjustable by means of an adjustable screw or a similar means in order to apply a pressure on the blood vessel. The cuff 101 can also be formed by a spring-loaded member and operated in a suitable manner such as hydraulically or pneumatically.

In FIG. 426 a cuff 101 in accordance with one embodiment of the present invention is shown in more detail. The cuff 101 comprises a number of segments 103 each adjustable and possible to tailor to fit a particular aneurism 102 of a blood vessel 104 to be treated. Each segment 103 can be adjusted either as a whole or individually. The segments 103 can be controlled and adjusted mechanically by an adjustable screw or similar or adapted to be filled with a fluid. For example, the segments can be provided axially along the blood vessel and also radially along the blood vessel forming a matrix of sub-segments that constitutes the cuff 101. In particular one segment can be located above and one below the aneurysm along the blood vessel.

The adjustment can be controlled by an electronic control unit 102 adapted to receive and transmit signals from a transmitter/receiver 200 located outside the body of a treated patient. The electronic control unit can also comprise a chargeable battery 111 chargeable from the outside by an external charger unit 112. The electronic control unit can comprise an electrical pulse generator 109 for generating electrical pulses as is described in more detail below.

The electronic control unit 102, such as a microprocessor or a MCU or a FPGA or a ASIC and can further be connected to or comprise a hydraulic pump 110 associated with a reservoir 115 containing of a fluid used to regulate the pressure of the cuff 101. The pump is thus adapted to pump the hydraulic fluid in or out from the cuff 101 in order to adjust the pressure applied in the aneurism. The control mechanism used for keeping the pressure in the cuff 101 can comprise a pressure tank 117.

In a preferred embodiment the pressure tank 117 is adapted to be able to change its volume still keeping substantially the same pressure, thus keeping the same pressure onto the aneurysm although some expansion of size of the aneurysm may occur. However, if the expansion goes too far the pressure tank may come out of range to keep the pressure constant and with some kind of volume detection in the pressure tank the pump 110 is then able to move fluid out from the pressure tank into the reservoir 115 to again be within pressure range in the pressure tank. The pressure tank is also able to even out the systolic pulses supplied to the aneurysmic wall.

The cuff 101 can be shaped in any desirable form to enable treatment of an aneurism wherever it is located. In accordance with one embodiment the cuff 101 is provided with at least one sensor 107 adapted to sense the pressure from the blood vessel that the cuff is surrounding.

The sensor(s) 107 used to generate a signal indicative of one or many parameters related to the aneurism and the device 101 used for treating the aneurism can for example be a gauge sensor. The sensor 107 can be adapted to generate sensor signals used for monitoring parameters including but not limited to the pressure in a hydraulic cuff, the pressure of a mechanical cuff, the pressure of a pneumatic cuff, the pressure in a blood vessel, the shape of the blood vessel in particular a parameter related to the diameter of the aneurysm.

An alternative or complement to the remote placed transmitter 200 is a switch (part of 102), preferable subcutaneously placed, such a switch may be mechanical or electrical, such as a microprocessor or a MCU or a FPGA or a ASIC, or the switch may comprise a small hydraulic control reservoir.

The restriction device may comprise any hydraulic device or mechanical device or stimulation device alone or monitoring/sensor device in any combination as described in the present application. The stimulation device may comprise both thermal stimulation or electrical stimulation. If a hydraulic system is used the hydraulic pump may in a system comprise an injection port (part of 110) for the injection of hydraulic fluid, preferable for calibration of hydraulic fluid. A subcutaneously place switch may also be used as well as a feed-back alarm system connected to the sensor/monitoring system.

Although the device has specific placements on the drawings it should be understood that the placement might vary.

Any combination of features or embodiments may comprise from any source within this application. Any embodiment in any combination that is disclosed in this application, specially, but not limited to, in FIGS. 425-433, may be used.

In FIG. 427 a view illustrating a mechanical cuff 101 is shown. The cuff can for example comprise an elastic material 301 kept in place by a suitable compressing device. The cuff 101 in accordance with one embodiment of the present invention comprises an elastic material in the form of a number of gel filled pads 301. The pads 301 can be shaped in a suitable manner and in particular formed to absorb the geometrical shape of the aneurysm. This can for example be achieved by providing pads with different tilting angles. The elastic material 301 can be kept in place by at least one adjustable fastening member 303. The fastening member 303 can for example be adjusted by a screw 305 or a similar device. By adjusting the fastening member 303 the pressure applied on the aneurysm can be controlled.

In FIG. 428, a view illustrating a mechanical cuff 101 is shown. The cuff can for example comprise an elastic band 401. The band 401 can be adjusted by an adjustor 403 to provide a higher or smaller pressure on the aneurysm.

In FIG. 429, a view illustrating a hydraulic cuff 101 is shown. The cuff can for example comprise implantable member 501 adapted to hold fluid. The member 501 is adapted to be placed in connection with a blood vessel having an aneurysm. The member can exercise a pressure on the aneurysm the blood vessel in response to the conditions of the fluid of the member 501. By filling the member with a fluid pressure can be applied onto the aneurysm in order to prevent or reduce an expansion the aneurysm when implanted in a patient thereby enabling postoperative treatment of the aneurysm. Further the treatment can be adjusted postoperatively by regulating the pressure using an implanted pressure regulator 503.

The pressure regulator can for example be formed by a pressure tank 503 implanted in the patient interconnected via a hose 504 with the member 501. The pressure tank can comprise an expandable reservoir 505 for storing superfluous fluid.

In FIG. 430, a view illustrating a hydraulic cuff 101 is shown. The cuff can for example comprise implantable member 601 adapted to hold fluid. The member 601 is adapted to be placed in connection with a blood vessel having an aneurysm. The member can exercise a pressure on the aneurysm the blood vessel in response to the conditions of the fluid of the member 601. By filling the member with a fluid pressure can be applied onto the aneurysm in order to prevent or reduce an expansion the aneurysm when implanted in a patient thereby enabling postoperative treatment of the aneurysm. Further the treatment can be adjusted postoperatively by regulating the pressure using an implanted pressure regulator 603.

The pressure regulator can for example be formed by a spring loaded tank 603 implanted in the patient interconnected via a hose 604 with the member 601. The spring 606 used to control the pressure of the tank and thereby indirectly the pressure applied by the cuff 101 on the aneurysm can be an adjustable spring in order to control the pressure.

In FIG. 431, a view illustrating a hydraulic cuff 101 is shown. The cuff can for example comprise implantable member 701 adapted to hold fluid. The member 601 is adapted to be placed in connection with a blood vessel having an aneurysm. The member can exercise a pressure on the aneurysm the blood vessel in response to the conditions of the fluid of the member 701. By filling the member with a fluid pressure can be applied onto the aneurysm in order to prevent or reduce an expansion the aneurysm when implanted in a patient thereby enabling postoperative treatment of the aneurysm. Further the treatment can be adjusted postoperatively by regulating the pressure using an implanted pressure regulator 703.

The pressure regulator can for example be formed by a pump 703 implanted in the patient on a hose 704 interconnecting a tank 705 with the member 701. The pump 703 is used to control the pressure of the member 703 by pumping fluid in and out of the member 701 and thereby controlling the pressure applied by the cuff 101 on the aneurysm.

By sensing the pressure from the blood vessel the cuff can be controlled to apply a correct pressure on the blood vessel thereby keeping the form of the blood vessel essentially constant. For example the pressure may vary over time as a result of changes in the wall of the blood vessel of surrounding tissue. Also the pressure will change as a function of the phase in which the heart is working. In other words the pressure will be different in a systolic phase as compared to a diastolic phase. By using a pressure sensor the pressure applied by the cuff 101 can be adapted to react to changes in the sensed pressure and apply a corresponding counter pressure. The sensor signals generated by the sensor(s) 107 of the cuff can also be used to trigger an alarm in response to the sensor signal indicating an expansion of the aneurism. In response to an alarm signal being generated the cuff can be automatically controlled to exercise a counter pressure on the blood vessel to counter or limit the expansion of the aneurism.

In yet another embodiment, electrodes 108 can be provided in the cuff. The electrodes can be connected to the electrical pulse generator, which is adapted to generate electrical pulses for stimulating the wall of the aneurism. The purpose of the electrical stimulation is to increase the tonus of the wall of the aneurism.

In FIG. 432, a stimulation device 801 for treating a vascular aneurysm of a human or mammal patient is shown. The device 801 comprises at least one implantable electrode 803 adapted to be placed in close connection to the aneurysm. The electrode is adapted to provide an electrical stimulation pulse on a wall portion of the aneurysm. The electrical stimulation pulse can for example be generated by a pulse generator 805. The pulse generator can be implanted in the patient.

In accordance with one embodiment the electrical stimulation device used for treating a vascular aneurysm of a human or mammal patient is connected to electrodes adapted to stimulate the wall of the aneurism at multiple stimulation points. The multiple stimulation groups may further be organized in different stimulation groups which can stimulated independently of each other. In accordance with one embodiment the electrical stimulation is performed with positive and or negative voltage stimulation pulses. In one embodiment the current used for stimulation of the aneurysm wall is kept essentially constant.

The sequence of electrical pulses used to stimulation the wall of the aneurysm can be applied with a predetermined periodicity having periods of no stimulation therein between during which periods without stimulation the wall of the aneurysm is allowed to rest. The electrical stimulation signal can also be Pulse Width Modulated to control the energy applied. In accordance with one embodiment the electrical stimulation is applied during the systolic phase to increase the tonus of the wall of the aneurysm. The systolic phase can be detected by the sensors 107 used to sense the pressure of the aneurysm as described above.

In accordance with one embodiment the stimulation can be controlled to be applied with a temporarily increased intensity and position during emergency situations when the aneurysm is detected to rapidly expands, to limit the expansion of said aneurysm.

In order to provide input for controlling the pressure and or to monitor the aneurysm a device 107 can be provided. In FIG. 433 a view illustrating a sensor 901 used when treating or monitoring a vascular aneurysm of a human or mammal patient is shown. The sensor 901 is placed in relation to a wall portion of the aneurysm for generating a signal corresponding to a parameter related to the aneurysm or the treatment of the aneurism. The signal generated by the sensor can be a signal corresponding to the size of the aneurysm and is accessible via a signal output 903. For example the signal can be indicative of the diameter of the aneurysm. In accordance with one embodiment of the he sensor is a gauge sensor. The sensor 901 can also be adapted to generate any output related to monitoring or treatment of the aneurysm. For example the sensor can be adapted to sense the resistance, capacitance, pressure, volume extension, flexure of a member in contact with the aneurysm.

### K: Removing Blood Clots in the Vascular System

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for removing blood clots in the vascular system of a patient. Examples of such devices for removing blood clots in the vascular system of a patient will now be described.

In the following detailed descriptions of blood clot removal devices will be given. In the drawing figures, like reference numerals designate identical or corresponding elements throughout the several figures. It will be appreciated that these figures are for illustration only and are not in any way restricting the scope of the invention. Thus, any references to direction, such as “up” or “down”, are only referring to the directions shown in the figures. Also, any dimensions etc. shown in the figures are for illustration purposes.

FIG. 436 shows a patient 1 having an implanted heart pump 2. The implanted heart pump 2 is connected to the left ventricle 3a of the patient's heart 3 by means of a first tube 2a. The heart pump 2 is also connected to the aorta, generally designated 4, of the patient 1 by means of a second tube 2b. In this way, during operation the heart pump supplements or replaces the blood pumping of the patient's heart 3.

A medical implant 100 in the form of a blood clot removal device 100 according to the invention is shown provided in the second tube 2b of the heart pump 2, i.e., in the tube leading to the aorta 4 of the patient 1. This means that part of the blood flow passageway provided by the second tube 2b is replaced by a blood flow passageway in the blood clot removal device 100. The blood clot removal device 100 is thus an artificial device insertable in an artificial blood vessel of the patient. The function of the clot removal device is to remove any blood clots in the blood transported by the second tube 2b. These blood clots are preferably moved to a place free inside the body of the patient. However, they could alternatively be collected in a collecting volume, such as a bag 10a connected to the blood clot removal device 100 for subsequent removal or storage. A preferred storage capacity of the bag 10a can be more than 100 milliliters, for example. The blood clot removal device is an artificial device but could be inserted directly into a blood vessel of the patient or connected between two ends of a blood vessel. The clot removal device is preferably insertable in a blood flow passageway of the patient via surgery and is placed in the patient's abdomen or thorax or cephalic or neck region or retroperitoneal or any limb of the patient.

The design of a first preferred embodiment of the blood clot removal device 100 will now be described in detail, with reference to FIGS. 437-439. FIG. 437 shows a sectional view wherein the blood clot removal device 100 is provided in the blood flow passageway provided by the second tube 2b. A filter 12 is provided across the blood flow passageway 14 formed in a housing 11 with the function of stopping potential blood clots brought forward in the second tube 2b by the blood flow, indicated by arrows in the figure. In this preferred embodiment, the filter 12 comprises a plurality of preferably equally spaced strips 12a of some suitable material, such as biocompatible metal or plastic. These strips 12a are preferably arranged mutual parallel.

The distance between two adjacent strips is small enough to stop any blood clots. Thus, the distance is preferably less than 2 millimeters, and even more preferably less than 1.0 millimeters, but if the object is to protect the brain from larger clots only the distance could be larger. Although the blood flow passageway 14 in the preferred embodiment has an essentially square cross-sectional shape, it will be realized that it can take any suitable shape, such as rectangular or circular.

By providing a plurality of strips 12a as a filter across the blood flow passageway 14, a laminar blood flow is achieved downstream of the filter, which is advantageous in a blood clot preventing perspective. The blood flow configuration can be further enhanced by giving the plurality of strips 12a a desired cross-sectional shape, although the rectangular shape shown in FIG. 39 will be adequate for most purposes.

A first piston 16 is provided movable in a direction essentially perpendicular to the direction of the blood flow passageway 14, i.e., essentially perpendicular to the direction of the blood flow. This first piston 16 is driven by some suitable actuator means, such as pressurized air, a solenoid arrangement, an electrical servo motor or the like. A motor could be used to build up a stored power that could be released very fast, one example being a spring. In the preferred embodiment, pressurized air acts as the actuator means, since by latching the piston by means of a suitable latching means for the piston, building up the air pressure, and subsequently releasing the piston, very high speed of the piston is achieved, with enables short cleaning times of the filter.

The outer end portion of the first piston 16, i.e., the end portion facing the blood flow passageway 14, is essentially flush with the wall of the blood flow passageway in a non-active state of the blood clot removal device 100. Also, the outer end portion is provided with a concave portion or recess 16a (exaggerated in the figures) in order to act as a blood clot capturing means, as will be explained below.

The strike range of the first piston 16 is such that it extends all way across the blood flow passageway 14, as will be explained below with reference to FIGS. 440-441. A number of channels 16b corresponding to the number of strips 12a is provided in the first piston 16 to accommodate the strips when the first piston is in an extended position.

The first piston 16 is also provided with a plurality of through holes 17 in the direction of the blood flow passageway. These through holes will allow blood to flow through the blood flow passageway also during a cleaning operation, as will be explained below with reference to FIG. 444.

A second piston 18 is provided across the blood flow passageway 14 from the first piston 16. Also this second piston 18 is movable in a direction essentially perpendicular to the direction of the blood flow passageway 14 and is biased in the direction thereof by means of a spring 18a, for example. Likewise, the outer end portion of the second piston is provided with a recess 18b similar to the recess 16a of the first piston 16.

The first and second pistons 16, 18, are sealed to the housing 11 by means of a respective sealing 20, such as an O sealing.

A preferred embodiment of the method according to the invention will now be described with reference to FIGS. 440-443, showing different operational steps of the above-described device. FIG. 440 is a view similar to that of FIG. 437. However, this figures shows the blood clot removal device 100 during operation, wherein blood clots, generally designated 22, have assembled on the filter 12.

In FIG. 441, the first piston 16 has moved linearly from the retracted starting position shown FIG. 440 to an extended position, wherein the outer end portion thereof is in contact with the second piston 18. Due to the recess 16a in the outer end of the first piston 16, the blood clots 22 have been assembled in the recess 16a, whereby they have been brought with the first piston 16 during the movement thereof.

In the step shown in FIG. 441, the blood clots are confined in the recess 16a between the first and second pistons 16, 18.

By moving the first piston 16 an additional distance from the position shown in FIG. 441, the second piston 18 is pushed against the force of the spring 18a to a fully retracted position, see FIG. 442.

The plurality of strips 12a is in this position fully received in a respective channel 16b in the first piston.

It is seen that the outer ends of the first and second pistons define an unobstructed cavity in which the blood clots are confined. It is thereby possible to remove these by some suitable means. One such means could be a third piston 24, which is movable in a direction perpendicular to both the direction of the blood flow passageway 14 and the direction of movement of the first and second pistons 16, 18. This third piston, the movement of which could be controlled by means of pressurized air, a solenoid, an electric motor etc., scrapes off the blood clots collected by the first piston 16 and moves them to a place outside of the blood clot removal device 100 and the blood flow passageway 14.

FIG. 444 shows a side view of the first piston 16 in a fully extended position, i.e., corresponding to the view of FIG. 443. It is here seen that in this position the through holes 17 will be aligned with the blood flow passageway 14, thereby allowing blood to flow there through also during cleaning of the filter 12.

FIG. 445 shows a cross-sectional view taken along line X-X of FIG. 443. It is here seen that the third piston 24 collects the blood clots 22 during a downward movement, indicated by an arrow in the figure. The clots are ejected from the blood clot removal device 100 when the third piston 24 has reached its lower end position, shown in FIG. 446.

Again with reference to FIG. 442, it will be realized that pressurized air can be used for ejecting the collected blood clots from the cavity formed by the first piston 16 and the second piston 18.

A medical implant 100 in the form of a clot removal system, generally designated 100 and comprising a clot removal device, now designated 10 when described in the system, as described above will now be described with reference to FIGS. 447 and 671-683.

The system of FIG. 447 comprises a blood clot removal device 10 placed in the abdomen of the patient. An internal energy source in the form of an implanted energy transforming device 1002 is adapted to supply energy consuming components of the blood clot removal device 10 with energy via a power supply line 1003. An external energy transmission device 200 includes a wireless remote control transmitting a wireless signal, which is received by a signal receiver incorporated in the implanted energy transforming device 1002. The implanted energy transforming device 1002 transforms energy from the signal into electric energy which is supplied via the power supply line 1003.

The system of FIG. 447 is shown in a more generalized block diagram form in FIG. 667, wherein the patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 38 operable by polarized energy also is implanted in the patient for reversing the blood clot removal device 10. The wireless remote control of the external energy transmission device 200 transmits a wireless signal that carries polarized energy and the implanted energy transforming device 1002 transforms the wireless polarized energy into a polarized current for operating the electric switch 38. When the polarity of the current is shifted by the implanted energy transforming device 1002 the electric switch 38 reverses the function performed by the blood clot removal device 10.

FIG. 669 shows an embodiment identical to that of FIG. 668, except that an operation device 1007 implanted in the patient for regulating the blood clot removal device 10 is provided between the implanted energy transforming device 1002 and the blood clot removal device 10. This operation device can be in the form of a motor 1007, such as an electric servo motor. The motor 1007 is powered with energy from the implanted energy transforming device 1002, as the remote control of the external energy transmission device 200 transmits a wireless signal to the receiver of the implanted energy transforming device 1002.

FIG. 670 shows an embodiment identical to that of FIG. 668, except that it also comprises an operation device is in the form of an assembly 1008 including a motor/pump unit 78 and a fluid reservoir 1010 is implanted in the patient. In this case the blood clot removal device 10 is hydraulically operated, i.e. hydraulic fluid is pumped by the motor/pump unit 1009 from the fluid reservoir 1010 through a conduit 1011 to the blood clot removal device 10 to operate the clot removal device, and hydraulic fluid is pumped by the motor/pump unit 1009 back from the blood clot removal device 10 to the fluid reservoir 1010 to return the clot removal device to a starting position. The implanted energy transforming device 1002 transforms wireless energy into a current, for example a polarized current, for powering the motor/pump unit 1009 via an electric power supply line 1012.

Instead of a hydraulically operated blood clot removal device 10, it is also envisaged that the operation device comprises a pneumatic operation device. In this case, pressurized air can be used for regulation and the fluid reservoir is replaced by an air chamber and the fluid is replaced by air.

FIG. 671 shows an embodiment comprising the external energy transmission device 200 with its wireless remote control, the blood clot removal device 10, in this case hydraulically operated, and the implanted energy transforming device 1002, and further comprising a hydraulic fluid reservoir 1013, a motor/pump unit 1009 and a reversing device in the form of a hydraulic valve shifting device 1014, all implanted in the patient. The motor of the motor/pump unit 1009 is an electric motor. In response to a control signal from the wireless remote control of the external energy transmission device 200, the implanted energy transforming device 1002 powers the motor/pump unit 1009 with energy from the energy carried by the control signal, whereby the motor/pump unit 1009 distributes hydraulic fluid between the hydraulic fluid reservoir 1013 and the blood clot removal device 10. The remote control of the external unit comprising an energy transmission device 200 controls the hydraulic valve shifting device 1014 to shift the hydraulic fluid flow direction between one direction in which the fluid is pumped by the motor/pump unit 1009 from the hydraulic fluid reservoir 1013 to the blood clot removal device 10 to operate the clot removal device, and another opposite direction in which the fluid is pumped by the motor/pump unit 1009 back from the blood clot removal device 10 to the hydraulic fluid reservoir 1013 to return the clot removal device to a starting position.

FIG. 676 shows an embodiment identical to that of FIG. 667, except that an internal control unit 56 controlled by the wireless remote control of the external energy transmission device 200, an accumulator 1016 and a capacitor 1017 also are implanted in the patient. The internal control unit 56 arranges storage of electric energy received from the implanted energy transforming device 1002 in the accumulator 1016, which supplies energy to the blood clot removal device 10. In response to a control signal from the wireless remote control of the external energy transmission device 200, the internal control unit 56 either releases electric energy from the accumulator 1016 and transforms the released energy via power lines 1018 and 1019, or directly transforms electric energy from the implanted energy transforming device 1002 via a power line 1020, the capacitor 1017, which stabilizes the electric current, a power line 1021 and the power line 1019, for the operation of the blood clot removal device 10.

The internal control unit is preferably programmable from outside the patient's body. In a preferred embodiment, the internal control unit is programmed to regulate the blood clot removal device to remove any blood clots from the vascular system and place the blood clots outside the vascular system repeatedly according to a pre-programmed time-schedule.

In accordance with an alternative, the capacitor 1017 in the embodiment of FIG. 672 may be omitted. In accordance with another alternative, the accumulator 1016 in this embodiment may be omitted.

FIG. 673 shows an embodiment identical to that of FIG. 667, except that a battery 1022 for supplying energy for the operation of the blood clot removal device 10 and an electric switch 1023 for switching the operation of the blood clot removal device 10 also are implanted in the patient. The electric switch 1023 is operated by the energy supplied by the implanted energy transforming device 1002 to switch from an off mode, in which the battery 1022 is not in use, to an on mode, in which the battery 1022 supplies energy for the operation of the blood clot removal device 10.

FIG. 674 shows an embodiment identical to that of FIG. 673, except that an internal control unit 56 controllable by the wireless remote control of the external energy transmission device 200 also is implanted in the patient. In this case, the electric switch 1023 is operated by the energy supplied by the implanted energy transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the blood clot removal device 10.

FIG. 675 shows an embodiment identical to that of FIG. 674, except that an accumulator 1016 is substituted for the battery 1022 and the implanted components are interconnected differently. In this case, the accumulator 1016 stores energy from the implanted energy transforming device 1002. In response to a control signal from the wireless remote control of the external energy transmission device 100800, the internal control unit 56 controls the electric switch 1023 to switch from an off mode, in which the accumulator 1016 is not in use, to an on mode, in which the accumulator 1016 supplies energy for the operation of the blood clot removal device 10.

FIG. 676 shows an embodiment identical to that of FIG. 675, except that a battery 1022 also is implanted in the patient and the implanted components are interconnected differently. In response to a control signal from the wireless remote control of the external energy transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the blood clot removal device 10.

Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the blood clot removal device 10.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the blood clot removal device 10 (mechanically operated).

FIG. 678 shows an embodiment identical to that of FIG. 676 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the blood clot removal device 10.

FIG. 679 schematically shows conceivable combinations of implanted components of the apparatus for achieving various communication options. Basically, there are the blood clot removal device 10, the internal control unit 102, motor/pump unit 1009, and the external energy transmission device 200 including the external wireless remote control. As already described above the wireless remote control transmits a control signal which is received by the internal control unit 102, which in turn controls the various implanted components of the apparatus.

A feedback device, preferably in the form of a sensor 1025, may be implanted in the patient for sensing a physical parameter of the patient, such as the pressure in a blood vessel. The internal control unit 102, or alternatively the external wireless remote control of the external energy transmission device 200, may control the blood clot removal device 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver.

Alternatively, the wireless remote control may comprise a signal receiver or transceiver and the internal control unit 102 may comprise a signal transmitter or transceiver. The above transceivers, transmitters and receivers may be used for sending information or data related to the blood clot removal device 10 from inside the patient's body to the outside thereof.

Alternatively, the sensor 1025 may be arranged to sense a functional parameter of the blood clot removal device 10.

Where the motor/pump unit 1009 and battery 1022 for powering the motor/pump unit 1009 are implanted, the battery 1022 may be equipped with a transceiver for sending information on the condition of the battery 1022.

FIG. 680 shows an alternative embodiment wherein the blood clot removal device 10 is regulated from outside the patient's body. The clot removal system 100 comprises a blood clot removal device 10 connected to a battery 1022 via a subcutaneous switch 1026. Thus, the regulation of the blood clot removal device 10 is performed non-invasively by manually pressing the subcutaneous switch, whereby the operation of the blood clot removal device 10 is switched on and off. It will be appreciated that the shown embodiment is a simplification and that additional components, such as an internal control unit, can be added to the clot removal system.

FIG. 681 shows an alternative embodiment, wherein the clot removal system 100 comprises a blood clot removal device 10 in fluid connection with a hydraulic fluid reservoir 1013. Non-invasive regulation is performed by manually pressing the hydraulic reservoir connected to the blood clot removal device 10.

A further embodiment of a system according to the invention comprises a feedback device for sending information from inside the patient's body to the outside thereof to give feedback information related to at least one functional parameter of the clot removal device or system or a physical parameter of the patient, thereby optimizing the performance of the system.

One preferred functional parameter of the device is correlated to the transfer of energy for charging the internal energy source.

In FIG. K682, an arrangement is schematically illustrated for supplying an accurate amount of energy to a clot removal system 100 implanted in a patient, whose skin SK is indicated by a vertical line.

A blood clot removal device 10 is connected to an implanted energy transforming device 200, likewise located inside the patient, preferably just beneath the patient's skin SK. Generally speaking, the implanted energy transforming device 1002 may be placed in the abdomen, thorax, muscle fascia (e.g. in the abdominal wall), subcutaneously, or at any other suitable location. The implanted energy transforming device 1002 is adapted to receive wireless energy E transmitted from an external energy source 200 a provided in the external energy transmission device 200 located outside the patient's skin SK in the vicinity of the implanted energy transforming device 1002.

As is well known in the art, the wireless energy E may generally be transferred by means of any suitable Transcutaneous Energy Transfer (TET) device, such as a device including a primary coil arranged in the external energy source 200 a and an adjacent secondary coil arranged in the implanted energy transforming device 1002. When an electric current is fed through the primary coil, energy in the form of a voltage is induced in the secondary coil which can be used to operate a clot removal device, e.g. after storing the incoming energy in an energy storing device or accumulator, such as a battery or a capacitor. However, the present invention is generally not limited to any particular energy transfer technique, TET devices or energy storing devices, and any kind of wireless energy may be used.

The amount of transferred energy can be regulated by means of an external control unit 200b controlling the external energy source 200 a based on the determined energy balance, as described above.

In order to transfer the correct amount of energy, the energy balance and the required amount of energy can be determined by means of an internal control unit 102 connected to the blood clot removal device 10. The internal control unit 102 may thus be arranged to receive various measurements obtained by suitable sensors or the like, not shown, measuring certain characteristics of the blood clot removal device 10, somehow reflecting the required amount of energy needed for proper operation of the blood clot removal device 10. Moreover, the current condition of the patient may also be detected by means of suitable measuring devices or sensors, in order to provide parameters reflecting the patient's condition.

Hence, such characteristics and/or parameters may be related to the current state of the blood clot removal device 10, such as power consumption, operational mode and temperature, as well as the patient's condition reflected by, e.g., body temperature, blood pressure, heartbeats and breathing.

Furthermore, an energy storing device or accumulator 1016 may optionally be connected to the implanted energy transforming device 1002 for accumulating received energy for later use by the blood clot removal device 10. Alternatively or additionally, characteristics of such an accumulator, also reflecting the required amount of energy, may be measured as well. The accumulator may be replaced by a battery, and the measured characteristics may be related to the current state of the battery, such as voltage, temperature, etc. In order to provide sufficient voltage and current to the blood clot removal device 10, and also to avoid excessive heating, it is clearly understood that the battery should be charged optimally by receiving a correct amount of energy from the implanted energy transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 is adapted to determine the energy balance and/or the currently required amount of energy, (either energy per time unit or accumulated energy) based on measurements made by the above-mentioned sensors or measuring devices on the blood clot removal device 10, or the patient, or an energy storing device if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal.

Alternatively, sensor measurements can be transmitted directly to the external control unit 200b wherein the energy balance and/or the currently required amount of energy can be determined by the external control unit 200b, thus integrating the above-described function of the internal control unit 56 in the external control unit 200b. In that case, the internal control unit 56 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements.

Hence, the present solution employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by the clot removal device. The clot removal device may use the received energy either for consuming or for storing the energy in an energy storage device or the like. The different parameters discussed above would thus be used if relevant and needed and then as a tool for determining the actual energy balance. However, such parameters may also be needed per se for any actions taken internally to specifically operate the clot removal device.

The internal signal transmitter 1027 and the external signal receiver 200c may be implemented as separate units using suitable signal transfer means, such as radio, IR (Infrared) or ultrasonic signals.

Alternatively, the internal signal transmitter 1027 and the external signal receiver 200c may be integrated in the implanted energy transforming device 1002 and the external energy source 200a, respectively, so as to convey control signals in a reverse direction relative to the energy transfer, basically using the same transmission technique. The control signals may be modulated with respect to frequency, phase or amplitude.

To conclude, the energy supply arrangement illustrated in FIG. K682 may operate basically in the following manner. The energy balance is first determined by the internal control unit 56. A control signal reflecting the required amount of energy is also created by the internal control unit 56, and the control signal is transmitted from the internal signal transmitter 1027 to the external signal receiver 200c.

Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

A method is thus provided for controlling transmission of wireless energy supplied to an electrically operable clot removal device implanted in a patient. The wireless energy E is transmitted from an external energy source located outside the patient and is received by an internal energy receiver located inside the patient, the internal energy receiver being connected to the clot removal device for directly or indirectly supplying received energy thereto. An energy balance is determined between the energy received by the internal energy receiver and the energy used for the clot removal device. The transmission of wireless energy E from the external energy source is then controlled based on the determined energy balance.

A system is also provided for controlling transmission of wireless energy supplied to an electrically operable clot removal device implanted in a patient. The system is adapted to transmit the wireless energy E from an external energy source located outside the patient which is received by an implanted energy transforming device located inside the patient, the implanted energy transforming device being connected to the clot removal device for directly or indirectly supplying received energy thereto. The system is further adapted to determine an energy balance between the energy received by the implanted energy transforming device and the energy used for the clot removal device, and control the transmission of wireless energy E from the external energy source, based on the determined energy balance.

The functional parameter of the device is correlated to the transfer of energy for charging the internal energy source.

In yet an alternative embodiment, the external source of energy is controlled from outside the patient's body to release electromagnetic wireless energy, and released electromagnetic wireless energy is used for operating the blood clot removal device.

In another embodiment, the external source of energy is controlling from outside the patient's body to release non-magnetic wireless energy, and released non-magnetic wireless energy is used for operating the blood clot removal device.

Those skilled in the art will realize that the above various embodiments according to FIGS. 667-683 could be combined in many different ways. For example, the electric switch operated polarized energy could be incorporated in any of the embodiments of FIGS. 669, 672-678, the hydraulic valve shifting device 1014 could be incorporated in the embodiment of FIG. 670, and the gear box 1024 could be incorporated in the embodiment of FIG. 669.

Wireless transfer of energy for operating the clot removal device has been described to enable non-invasive operation. It will be appreciated that the clot removal device can be operated with wire bound energy as well. On such example is shown in FIG. 683, wherein an external switch is interconnected between the external energy source 200a and an operation device, such as an electric motor regulating the blood clot removal device 10, by means of power lines. An external control unit 200b controls the operation of the external switch to effect proper operation of the blood clot removal device 10.

Methods relating to the above described clot removal device and system will now be described in detail.

The clot removal device can be placed surgically in the patient via a laparoscopic abdominal approach. First, a tube is inserted into the abdomen of the patient's body and this tube is used to fill the patient's abdomen with gas, thereby expanding the patient's abdominal cavity. At least two laparoscopic trocars are then placed in the patient's body, where after a camera is inserted through one of the trocars into the patient's abdomen. At least one dissecting tool is inserted through a trocar and dissection is performed at two intended areas of the patient. A clot removal device is placed in any part of the vascular system in the abdomen.

Alternatively, the clot removal device can be placed in the patient's thorax. Thus, a tube is inserted into the thorax of the patient's body and this tube is used to fill the patient's thorax with gas, thereby expanding the patient's thoraxical cavity. At least two laparoscopic trocars are then placed in the patient's body, where after a camera is inserted through one of the trocars into the patient's thorax. At least one dissecting tool is inserted through a trocar and dissection is performed at two intended areas of the patient. A clot removal device is placed in any part of the vascular system in the thorax.

An operation method for surgically placing a clot removal device starts with cutting the patient's skin and dissecting a placement area where to place the clot removal device inside the vascular system in the abdomen or thorax or retroperitoneal or subcutaneously or any limb of the patient. When a suitable place has been found, the clot removal device is placed in the placement area. The clot removal device can then be used postoperatively and non-invasively without penetrating the patent's skin for removing any blood clots from the vascular system to outside thereof, while using energy from an energy source without any penetration through the patient's skin to power the blood clot removal device.

A method for surgically treating a patient needing a blood clot removal device in the vascular system in the patient's abdomen preferably comprises cutting an opening in the patient's abdominal wall and then dissecting an area of the vascular system. A clot removal device is placed inside the vascular system, and the abdominal wall is sutured. In one embodiment, blood clots are moved away from the vascular system into an encapsulated closed bag in the patient's abdomen by means of the blood clot removal device. In another embodiment, blood clots are moved to the free abdomen.

Alternatively, a method for surgically treating a patient needing a blood clot removal device in the vascular system in the thorax comprises cutting an opening in the thorax wall and then dissecting the area of the vascular system. A clot removal device is placed inside the vascular system, and the thorax wall is sutured. A step of moving blood clots away from the vascular system can comprise moving blood clots either to a place that is free in the thorax, to a place that is free in the abdomen, or to a place that is encapsulated in a closed bag in the thorax.

In one embodiment, a method of using a system for removing blood clots comprises implanting an implantable source of energy, such as an implanted energy transforming device 1002 and an accumulator 1016, in the patient. An external source of energy, such as an external energy transmission device 200, is provided for providing energy to the system. This external source of energy is operated to release wireless energy, thereby non-invasively charging the implantable source of energy with the wireless energy, while controlling the implantable source of energy from outside the patient's body. In connection with operation of the clot removal device, energy is releasing. The wireless energy is preferably stored in the implantable source of energy.

During operation, the system for removing blood clots postoperatively and non-invasively regulates the clot removal device. Any blood clots, which have been accumulated in the vascular system of the patient's body, are moved away from the vascular system and are then placed outside the vascular system. This can be accomplished by an energy source, preferably repeatedly according to a pre-programmed time-schedule. The movement of any blood clots away from the vascular system and placement of the blood clots outside the vascular system are preferably repeated and at least partly controlled by an internal control unit getting input from a sensor sensing any physical parameter of the patient or any functional parameter of the device.

Preferred embodiments of a clot removal device, a system comprising a clot removal device, and a method according to the invention have been described. A person skilled in the art realizes that these could be varied within the scope of the appended claims.

The blood clot removal device has been described as an artificial device insertable in an artificial blood vessel of the patient. Alternatively, the blood clot removal device is an artificial device adapted to be placed between two open ends of a blood vessel of the patient or be placed inside or attached to a blood vessel via surgery.

The blood clot removal device has been described to be placed in the patient's abdomen or thorax. It could also be adapted to be placed in the patient's retroperitoneal region or cephalic or neck region or any limb of the patient. The filter in the blood clot removal device may be exchanged and replaced with a new fresh filter when it becomes dirty. One embodiment of such a solution is described below in FIGS. K1002 and K31.

In FIG. K448 a cassette 127 for holding filters is shown. The cassette 127 comprises a revolving cylinder 129 having segments 130 each holding a filter. The cylinder 129 is tightly sealed between two supports 131 holding the cylinder 129 in place and providing a tight sealing. Preferably, the contacting surfaces are made of ceramics to seal the surfaces with fine tolerances. The blood flow passageway of an implantable blood clot removal device passes through the cassette 127. The cassette is driven by a motor 133 causing the cylinder 129 to revolve at suitable times. Preferably, the filter is designed to move any collected blood clots out from the blood flow passageway together with revolving filter, when the filter leaves the blood flow passageway to be replaced with a new filter. The filter may be any kind of filter preferably with a space for the collected blood clots or blood clots adherent to the filter itself. Such a space to both the sealing plates when rotating, should preferably be larger before the filter seen in the blood flow passageway. The motor is powered by a power supply 123b. The power supply can be a power supply like the power supplies 123 or 123a. In accordance with one embodiment the power supplies 123, 123a and 123b is the one and same power supply. As with the power supplies 123 and 123a, the power supply 123b can receive wireless energy in a suitable form, including but not limited to inductive energy ultrasonic energy, light energy or any other form of wireless energy set out above. The energy is supplied by an external wireless energy transmitter 6 adapted to transmit energy through the skin SK of a patient having the cassette 127 implanted. The power supply 123b can also comprise a control unit as described above for controlling the revolving cassette 127. The control unit can provide feedback to the outside and receive input data from an external transceiver 200a in a manner similar to the control unit used in conjunction with control of the pump.

In FIG. 449 the cassette 127 is shown from the side with the supports 131 and the revolving cylinder 129 spaced apart is a disassembled view.

In FIG. 450a an alternative embodiment of the cassette 127 is shown. The view in FIG. K448a is similar to the view in FIG. 450a. In the embodiment in FIG. 450a a magazine 135 having a number of cylinders 129 stored therein is provided. Hereby a cylinder 129 can by replaced by shifting the cylinders in the magazine 135. In one embodiment the cylinders are shifted by pressurized air or a motor. The filter is then first replaced in the blood flow passageway and thereafter in a position outside the blood flow passageway replaced in the cassette. Such a replacement could preferably take place by having a number of filters in the cylinder 135 on one side of the cassette marked with 135 and moving the dirty filters out from the cassette into the cylinder on the other side of the cassette.

In an alternative embodiment the cylinder 135 is instead a cleaning device adapted to clean the filter at a position outside the blood flow passageway.

In FIG. 450b the cassette 127 is shown from the side with the supports 131 and the revolving cylinder 129 spaced apart is a disassembled view.

It should be noted that any embodiment or part of embodiment or feature or method or associated system or part of system described herein may be combined in any combination.

### L: Treating Urinary Retention of a Mammal

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for treating urinary retention of a mammal patient, examples of such devices for treating urinary retention of a mammal patient will now be described.

FIG. 451 is a general cross-sectional view of an implantable medical device 100 in the form of an apparatus for compressing the urinary bladder of the patient when implanted in a human patient.

Referring to FIG. 452 an embodiment of the apparatus is shown as implanted when operating on a urinary bladder 30. The apparatus 100 includes a powered member and a control device 102. The controls device 102 controls the operation of the powered member and is capable of receiving a signal from a sensor 150 related to the volume in the urine bladder such as a pressure sensor or any sensor related to the wall of the urine bladder (not shown and to communicate out from the body an alarm signal. The sensor is connected to sensor control unit 205 of the control device 102. Several different types of input sensors may be used determining for example stretching or bending or pressure in the urine bladder wall or for example sensing volume or pressure inside the urine bladder. Most likely these sensors are only indirect causing the bladder to be emptied by presenting an alarm for the patient informing that it is time to empty the bladder. Such an alarm may be generated audible or visually. A remote control 200 controlled from outside body of the patient in order to operate the powered member, such as a wireless remote control 200 communicating with an internal control unit 203 (not shown) or at least one implanted switch 204 (not shown). The control device 102 also includes an energy source 201 (not shown) for supplying energy consuming parts of the powered member with energy. The energy source can be wirelessly supplied from the outside from an energizer unit 400. For this purpose the control device 102 is provided with an energy transforming device 202. The control device comprises an external part 200 which is provided a manually operated switch 201A (not shown) and with an injection port 201B (not shown) for hydraulic fluids communicating with an internal reservoir 206 (not shown). The control device further includes a motor/pump function. It is contemplated that the features related to hydraulic fluid is relevant for a hydraulic embodiment of FIG. L4 and the medical device 100 includes a pressurizer 140 and a urinary bladder contact part 120 which may be fixated to the urinary bladder. The pressurizer includes an operation device 144 fixated to human tissue in this case the pubic bone and is operative connected to movable arm 142 connected to the contacting part. In operation to exert a pressure on the urinary bladder and thereby discharging urine through the urethra, the operation device 144 is activated by control device to move the arm towards the urinary bladder which thereby is contracted.

Further FIG. 452 shows a restriction device 59B for temporarily restriction of a ureter (this embodiment closes both ureters with restriction devices). The apparatus may eventually be provided with such restriction devices for the ureters which are controlled by the control device 102 to close the ureters when operating the powered member to discharge urine in order to prevent from a urinary flow from the bladder to the kidneys. In operation the control device 102 is activated and supplies the powered member with energy. The pressurizer will then actuate the urinary bladder to compress so the urinary pressure in the bladder is raise so urine is discharges through the urethra. When the urinary discharge is finalized the pressurizer alleviates the urinary and returns to its initial position, while the restriction devices for the ureters are released and the urinary bladder can receive urine from the kidneys. FIG. 453 shows the same apparatus as FIG. 452 when discharging urine through urethra. For this purpose the urinary sphincter 59C is deactivated an open and the restriction device 59B. The apparatus needs to exert a considerable pressure (about 60-80 cm water pressure) to force urine out from the bladder and urine may thereby backflow through ureters 32A, 32B with potential risks for damaging the kidneys. To prevent from any such complications, the control device is provided with restriction devices 59A, 59B arranged to temporarily contract the ureters and close them during the operation of discharging urine. The urine pressure in the ureter is normally around 50 cm water, however short term pressure increase is most likely not damaging the kidneys and therefore the restriction devices 59A and 59B may be omitted.

FIG. 454 shows schematically a variant of the pressurizer which now includes reservoir 440 that is hydraulically connected to a cavity 420 of the contacting part. A control device 102 controls the operation of the pressurizer in a similar way as explained with FIG. 452. When operating the apparatus to discharge urine the control device activates transportation of fluid from the reservoir 440 to the cavity 420 of the contacting part which thereby expands in volume so the urinary bladder compresses and urine is discharge through the urethra as a consequence of a raised urinary pressure in the bladder. In order to release the bladder, fluid is transported back to the reservoir from the cavity. The back transportation can either be performed by a powered operation (i.e. a pump operatively connected to the reservoir) or as result of the raising urinary pressure in the bladder. A second connection 444 between the cavity and the reservoir is used for the later transport. If the pump pumping capacity is larger than the flow capacity of said second connection the second connection may be opened all the time. FIG. 454 further sows a sensor 445 communicating with control device sensor control unit (not shown).

FIG. 455 shows schematically another variant of the pressurizer 540 including an operation device 544 attached to a support device 510 fixated to the urinary bladder wall. The pressurizer may be both hydraulically or mechanically operated. In this case a mechanical construction has an actuator 542 operably connected to the operation device to perform an actuating movement to actuate the contacting part 520 to compress the urinary bladder. In operation to discharge urine, the operation device performs a pivotal movement of the actuator so it contacts the contact part 520 to compress the urinary bladder in order to discharge urine through the urethra. When releasing the bladder the operation device removes the actuator 542 from the contacting part 520 to its initial position and the urinary bladder is ready to receive urine through the ureters.

FIG. 455 shows an embodiment of the apparatus of FIG. 452 with the operation device 544 placed on the abdominal wall as an alternative support function. FIG. 455 shows another alternative of the apparatus of FIG. 452 with the operation device supported another bone structure. FIG. 455 shows an alternative of the apparatus of FIG. 452 without restriction devices for the ureters and without a urinary sphincter function.

Some patients having urinary retention also have urinary incontinence. In such a case a separate urinary sphincter 59C is included in the system, a restriction device closing the urethra until the patient wants to urinate. In such a case lower pressure is needed to empty the bladder because the no force would be needed to open the sphincter by intra bladder pressure. In this case the ureter restriction devices may be omitted.

The reservoir may be placed anywhere inside the body, however preferable in the abdominal cavity, maybe placed onto the urine bladder or in the pelvic region. The amount of liquid in the reservoir may be calibrated with fluid by using an injection port placed inside the body within reach from a special injection port needle. The reservoir may also be omitted and only the injection port may be used to fill and empty the expandable member.

By reference to FIG. 456 and FIG. 457a the apparatus has an expandable member 20 with a cavity for accommodating hydraulic fluid that is placed inside the urinary bladder 30 which contains urine arrived from the ureters 32A, 32B. A control device 102 operates the expansion and thereby the volume of the expandable member. The control device 102 is connected to a bladder operating reservoir 54 for hydraulic fluid which is connected to the expandable member with an interconnecting device 56 for transporting hydraulic fluid between the bladder operating reservoir 54 and the expandable member 20.

A pump 53 is supporting the fluid transportation. The interconnecting device 56 is a tube-shaped device surgically incised through the wall of the urinary bladder and attached thereto with tunneling technique whereby the bladder wall is sutured to itself. The interconnecting device is supported by the net 58 which seals fixates by admitting tissue in-growth. In FIG. L457a the interconnecting device 56 is attached to a detachable coupling 55 that attaches the bladder operating reservoir with the expandable member as will be described below with FIG. 457b. The control unit 102 is located in the patient and includes a number of functional elements necessary for operating the apparatus, such as an operating pump 529 for the hydraulic fluid, a source of energy 521 for driving the operating pump and other energy consuming parts of the apparatus. An external energizer 60 transfers wireless energy to an energy transforming device 522 so the source of energy 521 can be supplemented. An external control unit 200 provides wireless communication with an internal control unit 102 for operating the apparatus. Also, the pressure sensor 57 is connected to a sensor control function 524 of the control unit 102. The control unit 102 is placed in a second portion 141″ of a remote unit and includes the mentioned functions and a first portion 141′ of the remote unit includes an injection port 521B and a manually operable switch 522B. One or more parts of the control device may be implanted subcutaneously or in the abdominal cavity or the pelvic region or any other suitable place inside the body. The embodiment depicted in FIG. L457a is adapted for a patient suffering from a complication where the urinary sphincter is permanently closed. For this reason, the expandable member 20 of the apparatus needs to exert a considerable pressure (about 60-80 cm water pressure) to force urine out from the bladder and urine may thereby backflow through ureters 32A, 32B with potential risks for damaging the kidneys. To prevent from any such complications, the control device is provided with restriction devices 59A, 59B arranged to temporarily contract the ureters and close them during the operation of discharging urine with the expandable member. The restriction devices are operated from the control assembly in manner to perform their temporary contraction during the discharge performance. Suitable mechanical or hydraulically operated restriction devices and their control are described in more detail in European Patents Nos. EP 1253880; EP 1284691; and EP 1263355. The urine pressure in the ureter is normally around 50 cm water, however short term pressure increase is most likely not damaging the kidneys and therefore the restriction devices 59A and 59B may be omitted.

When the pump 53 is not pumping to fill the expandable member and if the passage-way 56 between the bladder operating reservoir and the expandable member is free, then the expandable member is emptied by urine filling the bladder. Another alternative is that the pump 53 starts in steps to empty the expandable member for example pressure controlled or controlled by any other input sensor as mentioned elsewhere. A second connection 56B is introduced between the expandable member 20 and the bladder operating reservoir 54. The second connection is adapted to admit transportation of fluid from the member 20 to the bladder operating reservoir when the connection is closed. If the pumping volume capacity is significantly much larger than the emptying capacity of the second connection this connection may always stand open, also when the pump 53 transports fluid from the bladder operating reservoir 54 to the member 20. Introduction of the second connection shall be regarded as an optional alternative of the apparatus.

FIG. 457b is closer view of the detachable coupling 55 in FIG. 457a and its two mating parts 55A and 55B. A first mating part 55A is a part of the control device 50 and is connected to the bladder operating reservoir 54. The second mating part 55B is arranged on the expandable member 20. The two mating parts are readily attachable and detachable in order to conveniently attach or detach the expandable member to the control device 50. Accordingly, the expandable member becomes readily replaceable by intervention through the urethra with a suitable instrument. For this purpose the expandable member is capable of assuming an essentially cylindrical elongated shape to conveniently pass through the urethra. FIG. 457b also shows the second connection 56B as a part of the detachable coupling.

By reference to FIG. 457a and FIG. 458, the apparatus in operated by activating the operating pump of the control assembly 52 which is operable in response from a signal from a remote control 70.

The control assembly can also be connected to a pressure sensor 57 for monitoring the urinary pressure of the bladder. Several different types of input sensors may be used determining for example stretching or bending or pressure in the urine bladder wall or for example sensing volume or pressure inside the urine bladder. Most likely these sensors are only indirectly causing the bladder to be emptied by presenting an alarm for the patient informing that it is time to empty the bladder. Such an alarm is generated audible or visually. The remote control 70 may control a subcutaneous switch 525 for controlling the emptying of the bladder or communicating via the body used as a wire or with wireless communication. The pump now transports hydraulic fluid from the bladder operating reservoir 54, through the interconnection device 56 to the cavity of the expandable member 20, which thereby increases in volume in the urinary bladder and discharges urine through the urethra at a pressure that overcomes the closing force of the urethral sphincter, so voiding of the urinary bladder is accomplished.

During this operation the control assembly operates to close restriction device 59A, 59B to prevent any urinary backflow in the ureters. When the discharging performance is finished and the operating pump is inactive, the restriction devices 59A, 59B are released so urine can refill the urinary bladder. By the pressure of the refilled urine, the expandable member 20 subsequently collapses to retain a shape as shown in FIG. 457A when ready for a new performance as monitored by the pressure sensor.

Some patients having urinary retention also have urinary incontinence. In such a case a separate urinary sphincter is included in the system, a restriction device closing the urethra until the patient wants to urinate. In such a case lower pressure is needed to empty the bladder because the no force would be needed to open the sphincter by intra bladder pressure. In this case the restriction devices 59A and 59B may be omitted.

The bladder operating reservoir 54 may be placed anywhere inside the body, however preferable in the abdominal cavity, maybe placed onto the urine bladder or in the pelvic region. The amount of liquid in the bladder operating reservoir may be calibrated with fluid by using the injection port 521B and a subcutaneous reservoir 526 placed inside the body within reach from a special injection port needle. The subcutaneous reservoir may also be omitted and only the injection port may be used to fill and empty the expandable member. With the described embodiment it is also conceivable to control the duration/force of the urine discharge process, e.g. that data from the pressure sensor measuring the urinary pressure or easier the pressure inside the expandable member in the bladder controls the operation pump by logic in the control assembly. It should be noted that the expandable member may be elastic or only flexible, within the used pressure inside the same. FIG. 458 shows the apparatus of FIG. 452 (without the control unit 102) when discharging urine. The restriction devices 59A, 59B now close the ureters 32A, 32B while urinary sphincter 59C is open. FIG. 459 shows the apparatus of FIG. 458 when the urinary bladder is being refilled with urine and hydraulic fluid is returned to the bladder operating reservoir 54. The restriction devices 59A, 59B are now open while urinary sphincter 59C is closed. FIG. 459 also shows an embodiment wherein the restriction devices for ureters are hydraulically operated with hydraulic fluid from a special section of the bladder operating reservoir. The hydraulic fluid for operating the restriction devices can be displaced from the reservoir when the remaining section of the reservoir is filled as a consequence of the urinary pressure exerted on the expandable member. FIG. 460 illustrates another embodiment of the apparatus of FIG. 457a. Here the bladder operating reservoir 54 is hydraulically connected to a pump 527 in the control assembly 52 which operates to pump hydraulic fluid to the expandable member 20.

### M: Breast Implant System

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a breast implant system. Examples of such breast implant systems will now be described.

FIG. 461A shows very schematically a vertical cross-sectional view of a medical implant 100 in the form or a breast implant 100 according to a first embodiment. The breast implant comprises a first element 1, and a second element 2, each forming part of the breast implant 100 to be implanted in the breast area of a patient. In this embodiment, the first and second element 1, 2 are fluid chambers. The first and second fluid chambers 1, 2 are fixedly mounted to a rigid back plate 3 with a contour adapted to be placed adjacent the patient's thorax. A fluid line 4 connects the first and second fluid chambers 1, 2 and runs, in the embodiment shown, through the rigid back plate 3. A pump 5 is included in the fluid line 4 and is shown only very schematically. The pump 5 can have many different forms and can be of any suitable type. Also, the actual manner of driving the pump, such as manually or automatically by means of a motor, is of no particular importance and can be chosen appropriately. In the embodiment shown, the pump 5 includes a movable piston in a third fluid chamber 7, the one end of which is connected via the fluid line 4 to the first fluid chamber 1 and the other end of which is connected via the fluid line 4 to the second fluid chamber 2. Using the pump 5, fluid can be pumped and, thus, “exchanged” between the first and second fluid chambers 1 and 2.

In FIG. 461A the fluid chamber 1 is filled with fluid almost to its maximum capacity, so that the overall breast implant 100 is relatively sturdy. FIG. 461B shows the same breast implant 100 with some fluid being removed from the first fluid chamber 1 to the second fluid chamber 2 using the pump 5 (not shown in FIG. 461B). In this state the breast implant 100 is relatively flaccid. FIG. 461C shows the same breast implant 100 with the second fluid chamber 2 being filled almost to its maximum. The volume of the fluid chamber 1 is accordingly decreased. In this case, again, the breast implant 100 is relatively sturdy and is lifted more above the rigid back plate 3 as compared to the state shown in FIG. 461A. The sturdiness of the breast implant 100 in the state shown in FIG. 461C results partly from the fact that a pressure will build up in the second fluid chamber 2 as the volume of the second fluid chamber 2 reaches its maximum capacity.

FIG. 462A shows a simplified structure of a cross-sectional view through a breast implant 100 according to a second embodiment. Unlike FIG. 461, FIG. 462A shows a cross section taken horizontally through the breast implant 100. A rigid back plate is not provided in this embodiment, but can be provided if desired. The breast implant 100 comprises one first fluid chamber 1 and two second fluid chambers 2. More fluid chambers 1 and more or fewer fluid chambers 2 can also be present. In this embodiment, the fluid chambers 1 and 2 are separated by separating walls 8 made from a polymer material. The separating walls 8 are flexible, but preferably non-stretchable. The outer wall 9 of the fluid chambers 1, 2 is also flexible and preferably stretchable. Valves 11 are provided in the separating walls 8 to allow fluid to be exchanged between the fluid chambers 1, 2. These valves 11 are designed as pressure relief valves and can be of many different types. The purpose of the valves 11 is to allow fluid to flow from one fluid chamber to the next fluid chamber when a predetermined pressure difference is exceeded.

In order to allow fluid to flow through the same valve in both directions, the valves 11 are formed as two-way pressure relief valves. A very simple way of providing such two-way pressure relief valve is shown in FIG. 464c. Accordingly, there is a slit 12 in the flexible separating wall 8 which opens when a certain pressure difference between the adjacent fluid chambers is exceeded. FIG. 462A shows a “medium” state of the breast implant 100. However, FIG. 462A also shows by dotted lines one possible extreme state of the breast implant 100. That is, when the pressure in the second fluid chambers 2 is increased, such as by the patient manually compressing the second fluid chambers 2, fluid will flow into the first fluid chamber 1, as shown by the arrows. Then, when the patient releases the pressure on the second fluid chambers 2, the breast implant 100 will assume the shape as shown by the dotted lines.

FIG. 462B shows another extreme state of the breast implant 100 of FIG. 462A. In this case, when the pressure in the first fluid chamber 1 is increased, fluid is made to flow into the adjacent second fluid chambers 2, as indicated in FIG. 463B by the arrows. Once the pressure is released, the breast implant 100 will take the shape as shown in FIG. 462B by the dotted lines. Accordingly, the patient can easily change the shape of the breast implant 100 between the three states shown in FIGS. 462A and 463B. Additional intermediate states can also be achieved and even other forms can be achieved, for instance when only one of the two second fluid chambers 2 is compressed to urge fluid into the neighboring first fluid chamber 1.

FIGS. 465A and 465B show the breast implant 100 of FIGS. 462A, 463B covered on the outside with a layer 13 of soft material according to a third embodiment. The layer 13 may be formed by a liquid or gel type silicone or by a foam or a combination thereof. Also, bubbles of air or collagen may be incorporated in the silicone, foam or other soft material. The compartment forming the layer 13 is completely separate from the interconnected first and second chambers 1, 2. Again, a rigid back plate 3 is provided in this embodiment, but can be dispensed with if desired. FIGS. 465A and 465B demonstrate how such outer layer 13 can level out irregularities of the fluid chamber walls. More importantly, however, the outer layer 13 forms a barrier between the flexible, stretchable inner fluid chambers 1, 2 and any fibrosis that might form on the outside of the breast implant.

FIGS. 466A and 467B show a fourth embodiment of a breast implant. Again, a rigid back plate 3 is provided to which a first fluid chamber 1 and a second fluid chamber 2 are fixedly mounted. By means of the pump 5 which—in the embodiment shown—is again integrated in the rigid back plate 3, fluid can be exchanged between the first and second fluid chambers 1, 2. The first fluid chamber 1 together with the back plate 3 defines an enclosed space forming a third fluid chamber 14. The third fluid chamber 14 comprises a compressible medium, such as a gas or a foam in which the gas is contained.

Fluid is exchanged only between the first and second chambers 1, 2, whereas the third fluid chamber 14 is completely isolated, being separated from the first fluid chamber by separating wall 15 and from the second fluid chamber 2 by separating wall 16. Both separating walls 15, 16 are flexible and at least the separating wall 15 should be non-stretchable.

When fluid is pumped from the first fluid chamber 1 into the second fluid chamber 2, the volumes of the fluid chambers 1 and 2 will change accordingly, as is shown in FIG. 464B. The outer wall of the first fluid chamber 1 is elastic—in the embodiment shown—so as to adapt to the reduced volume, but may also be non-elastic, provided that it is sufficiently flexible to conform to the changed volume. Again, an outer layer 13, as shown in FIGS. 465A, 465B, can be provided here (and in all embodiments described herein). Due to the fact that the separating wall 15 is non-stretchable, the increased volume of the second fluid chamber 2 causes the pressure in the third fluid chamber 14 to rise from an initial pressure P1 to a raised pressure P2. Altogether, not only has the shape of the breast implant 100 dramatically changed, but the volume has also changed. However, the mass and, thus, the weight of the breast implant has not changed at all.

In the embodiment shown in FIGS. 466A, 467B, the elastic, non-stretchable wall 15 surrounding the third fluid chamber 14 may be replaced with a rigid wall of constant form. A fifth embodiment similar to the one shown in FIGS. 466A, 467B, but with a rigid separating wall 15, is shown in FIGS. 468A, 468B. The outer wall 17 of the first fluid chamber 1, which in this particular embodiment constitutes the outermost wall of the breast implant 100, comprises pleat-like folds 18. The pleat-like folds are trapezoidal in cross section in order to allow the folds 18 to be expanded and compressed irrespective of any fibrosis that has formed on the outer surface 17. Thus, due to these folds 18, fibrosis forming on the breast implant 100 will not hinder the enlargement of the breast implant 100.

It should be understood that the membrane 16 separating the incompressible fluid in the second chamber 2 from the compressible fluid, such as gas, in the third chamber 14 can be dispensed with in cases where there is no danger of compressible fluid from the gas chamber 14 reaching the first chamber 1. For instance, if the patient is lying down when the incompressible fluid is being re-transferred from the gas chamber 14 to the first fluid chamber 1, this would be a safe way to prevent any gas from being transferred from the gas chamber 14 to the fluid chamber 1.

While FIGS. 461 to 465 relate to breast implants with constant volume and weight but variable shape, and FIGS. 466A—468B relate to embodiments with a constant weight but variable shape and volume, the following embodiments relate to breast implants where the variable shape and volume involves a weight change of the breast implant. More specifically, at least one of the second fluid chambers 2 is implanted in the patient's body remote from the breast implant 100 and is connected to the first fluid chamber or chambers 1 in the breast implant 100 via one or more fluid lines 19. An example is shown in FIGS. 469A and 469B. FIGS. 469A and 469B show as a sixth embodiment a cross-sectional view through the breast implant 100 similar to the cross-sectional view in FIG. 461. In the specific embodiment shown, an outer layer 13 similar to the outer layer 13 in FIG. 465 is provided as an option.

An angular frame 20 to be placed close to the patient's thorax provides stability to the breast implant 100. The first fluid chamber 1 is fixedly connected to such frame 20. By removing fluid from the first fluid chamber 1 in the breast implant 100 into the remotely implanted second fluid chamber 2, one can change the look and feel of the breast implant 100 from sturdy to flaccid, as can be seen in FIGS. 469A and 469B.

FIGS. 469A and 469B only show the principle of the system. Not shown is the exact location of the second fluid chamber 2 in the patient's body, nor the specific way of exchanging the fluid between the first and second fluid chambers 1, 2. As to the location of implantation in the patient's body, the second fluid chamber 2 may be implanted in the patient's abdominal cavity or in the patient's chest area. More specifically, it may be placed outside the patient's thorax, in particular under the pectoralis muscle. The latter location can be easily reached during the surgery when the breast implant 100 is implanted, and this location does not disturb the patient very much.

Alternatively, the second fluid chamber 2 may be arranged subcutaneously so that it is easily accessible from the outside of the patient's body. The patient can then compress the second fluid chamber 2 in order to urge fluid into the first fluid chamber 1 of the breast implant 100, whereas the return flow into the remotely implanted second fluid chamber 2 can be achieved, e.g. by manually compressing the breast implant 100. Many variants of regulating the fluid flow between the first and second fluid chambers 1, 2 are conceivable, and one of those options will be described in the following with respect to another embodiment.

FIGS. 470A and 470B show a seventh embodiment of a breast implant 100 including a servo system. Apart from the servo system, one variant of regulating the fluid flow between the first and second fluid chambers 1, 2 by means of a subcutaneously implanted fluid chamber will also be described in the context of this embodiment.

More specifically, the second fluid chamber 2, which is interconnected with the first fluid chamber 1 in the breast implant 100 and implanted in the patient's body remote from the breast implant 100, has the form of a bellows. Operatively connected to the bellows 2 is a third fluid chamber comprising two sub-chambers 21 and 22. The sub-chamber 21 cooperates with the bellows 2 such that filling of the sub-chamber 21 with a fluid from the sub-chamber 22 will cause the bellows 2 to extend. The situation is such, however, that the volume change in the sub-chamber 21, which is also in the form of a bellows, is less than the volume change in the bellows 2. In turn, when fluid is removed from the sub-chamber 21 into the sub-chamber 22, the length and, thus, the volume of the second fluid chamber 2 decreases.

The way of obtaining a sturdy breast implant is shown in FIG. 470A. That is, when the patient has a subcutaneously arranged pressure chamber 23 in order to open a valve 24 to allow fluid to flow between sub-chambers 21 and 22, a preloaded spring 25 will cause the second fluid chamber 2 to decrease, thereby urging not only fluid from the sub-chamber 21 to flow to the sub-chamber 22 but also fluid from the second fluid chamber 2 to flow into the first fluid chamber 1 of the breast implant 10. The breast implant 10 will balloon accordingly. When pleased with the amount of ballooning of the breast implant, the patient will simply stop pressing the pressure chamber 23, so that the valve 24 closes.

When desiring to put the breast implant 10 back into a flaccid state, the patient can simply compress the subcutaneously implanted sub-chamber 22. This is shown in FIG. 467B. The increased pressure in the sub-chamber 22 will cause the valve 24, which is designed as a pressure relief valve, to open so that fluid flows from the sub-chamber 22 into the sub-chamber 21. The sub-chamber 21 will expand accordingly against the force of the spring 25. This will in turn cause the second fluid chamber 2 to expand also, and fluid will be drawn from the fluid chamber 1 of the breast implant 100 into the remotely implanted fluid chamber 2 of the servo system.

With the servo system shown in FIGS. 470A and 470B, the subcutaneously implanted sub-chamber 22 can be kept relatively small so that it will not disturb the patient's appearance too much. As a negative side effect, the pressure that the patient has to apply to the sub-chamber 22 in order to overcome the force of the spring 25 is relatively high. However, if the spring load is kept small, this has the effect that the inflation of the first fluid chamber 1 in the breast implant 100 by automatic action of the spring takes somewhat longer.

FIGS. 471A and 471B show a different servo system. The difference here is that the second fluid chamber 2 is not remotely implanted but forms part of the breast implant 100. The breast implant 100 comprises a rigid back plate 3 and a relatively stiff, but nevertheless flexible separating wall 26 separating the first and second fluid chambers. The separating wall 26 has openings 27 to allow fluid to be exchanged between the first and second fluid chambers 1, 2. The separating wall 26 is fixedly connected to the rigid back plate 3, with its normal position shown in FIG. 471A.

In the servo system shown in FIGS. 471A and 471B the sub-chamber 21 is again in the form of a bellows which extends between the rigid back plate 3 and the relatively stiff but nevertheless elastic separating wall 26. When the sub-chamber 21 is filled with fluid from the sub-chamber 22, the sub-chamber 21 will expand lengthwise, thereby urging apart the relatively stiff separating wall 26 and the rigid back plate 3. This in turn will cause fluid to flow from the first fluid chamber 1 through the separating wall 26 into the second fluid chamber 2. As a result, the shape of the breast implant 100 changes, which change is shown in FIG. 470B somewhat exaggerated. Again, the manner of controlling fluid to flow between the two sub-chambers 21 and 22 may be user-defined and realized e.g. in the same manner as described in relation to FIGS. 470A and 470B. The spring force required to compress the sub-chamber 21 in order to generate a return flow from the second fluid chamber 2 into the first fluid chamber 1 is provided by the elasticity of the relatively stiff separating wall 26, due to the fact that it is fixedly connected to the rigid back plate 3, similar to the functioning of a bow (of a bow and arrow).

FIGS. 472A to 472B show a ninth embodiment with a plurality of first chambers 1 in the breast implant 100. The first chambers 1 only fill a part of the breast implant 100, whereas the major part of the breast implant is filled with a different material, such as a foam or a silicone or a combination thereof.

Artificially produced collagen can also be used to fill the breast implant 10. The second fluid chamber 2 is again implanted subcutaneously here. As becomes clear from FIG. 472A, a little amount of fluid transferred from the second fluid chamber 2 into the first fluid chambers 1 of the breast implant will cause a substantial change of the breast implant's shape. FIG. 472B shows the shape of the breast implant 100 with fully inflated first fluid chambers 1. By manual compression of the breast implant 100, as indicated by arrows P in FIG. 472B, the fluid in the first fluid chambers 1 of the breast implant 100 can be urged back into the remotely implanted second fluid chamber 2, in order to regain the flaccid state shown in FIG. 472A.

A two-way non-return valve 28 may be placed in a line connecting the first and second fluid chambers 1, 2. The two-way non-return valve 28 is schematically shown in more detail in FIG. 472C.

FIG. 473 shows a tenth embodiment to demonstrate that the breast implant 100 may comprise a plurality of first fluid chambers 1 in an arbitrary arrangement, and a plurality of second fluid chambers 2 in fluid connection with the first fluid chambers 1 may be provided remote from the breast implant 100. In particular, the second fluid chambers 2 may be implanted subcutaneously for direct manual use by the patient. A second fluid chamber 2 may be connected to one or more of the first fluid chambers 1. Likewise, a first fluid chamber 1 may be connected to one or more of the second fluid chambers 2. This permits the design of a breast implant 100 according to very personal needs.

FIG. 474 shows a more complex breast implant system. The basic structure of the breast implant system corresponds to the structure described above in relation to FIGS. 472A and 472B, but could also be completely different. What is important in the eleventh embodiment of FIG. 474 is a pump P driven by a motor M and arranged to pump fluid between the first and second fluid chambers 1, 2. The fluid chamber 2 may be implanted anywhere in the patient's body, such as in the abdominal cavity.

The motor M is energized with wirelessly transmitted energy. For this purpose, the breast implant system comprises an energy transmitter 29 outside the patient's body and an energy transforming device 30 inside the patient's body, preferably subcutaneously implanted, to transform the wireless energy into electric energy. While it is possible to make use of a motor M adapted to directly transform the wirelessly transmitted energy into kinetic energy, or, alternatively, to use the wirelessly transmitted energy transformed into electric energy by means of the energy transforming device 30 to drive the motor M as the energy transforming device transforms the wireless energy into the electric energy, the specific embodiment shown in FIG. 474 first stores the transformed electric energy in an energy storage means E, before it is supplied to the motor M. Of course, it is also possible that a part of the transformed electric energy is directly used by the motor while another part of the transformed electric energy is stored in the energy storage means E. The energy storage means E may include an accumulator such as a rechargeable battery and/or a capacitor. It is less convenient, but possible, to implant a regular battery as the energy storage means E. But a regular battery may be used as the energy source to provide the wireless energy to be transmitted from outside the patient's body.

The breast implant 100 shown in the specific embodiment of FIG. 474 further includes a control unit. The control unit here comprises a first part C1 to be used by the patient from outside the patient's body and a second part C2 to be implanted inside the patient's body. Data can thus be transmitted wirelessly between the first and second parts C1, C2 of the control unit. In addition or alternatively, the implantable second part C2 of the control unit may be programmable via the first part of the control unit. Preferably, the data are transmitted between the first and second parts C1, C2 of the control unit in the same manner as energy is transmitted, such as via the elements 29 and 30.

The external part C1 of the control unit may also be replaced with a simple manually operable switch for activating the implantable control unit C2. Such switch is then arranged for subcutaneous implantation so as to be operable from outside the patient's body. It is also possible to combine the switch with an external part C1 of the control unit.

Furthermore, feedback information may be sent between the implanted part C2 and the external part C1 of the control unit. Such feedback information may include information related to the energy to be stored in the energy storage means E. The control unit can make use of such feedback information for adjusting the amount of wireless energy transmitted by the energy transmitter 29. The feedback information may be related to an energy balance, which may be defined either as the balance between an amount of wireless energy received inside the human body and an amount of energy consumed by the motor and pump, or as the balance between a rate of wireless energy received inside the human body and a rate of energy consumed by the motor and pump.

FIG. 474 shows an injection port 31 implanted under the patient's skin. Fluid can be added to or removed from the breast implant system by means of a regular syringe if need arises. While only one injection port 31 is shown in FIG. 474, two or more injection ports may be provided in order to allow for individual adjustment of the fluid volume in the particular fluid chambers. The injection port 31 can also be used to equalize a pressure difference between one or more of the fluid chambers.

In context with a twelfth embodiment of a breast implant system, FIGS. 475A and 475B show a manner of implanting the second fluid chamber or chambers remote from the breast implant under the pectoralis muscle. The breast implant 100 is here formed from a single first chamber 1, but can as well comprise more than one first fluid chamber 1. It is designed to increase the volume of a natural breast 50, but can likewise be designed to replace an amputated breast. The second fluid chamber 2 (or fluid chambers) serves as a reservoir for the first fluid chamber and is implanted remote from the first fluid chamber 1 underneath the patient's minor pectoralis muscle 40 next to the patient's thorax. A pump 5 is also implanted remote from the breast implant 100 to exchange fluid between the first and second fluid chambers 1, 2. The pump 5 may be combined with a motor, control unit and other parts of the systems previously described. Instead of or in addition to the pump 5, other elements of the breast implant system embodiments described above may be combined with this twelfth embodiment, such as remotely implanted elements for manual operation by the patient, pressure relief valves and so forth.

In this twelfth embodiment, the second fluid chamber 2 is wide and flat as compared to the first fluid chamber 1, i.e. it has a substantially larger surface-to-volume ratio than the first fluid chamber 1.

This allows a substantial subjective volume change of the patient's breast 50 to be achieved by pumping fluid between the first and second fluid chambers 1, 2, as can be seen from a comparison of FIGS. 475A and 475B showing the breast implant system with differently filled first and second fluid chambers 1, 2.

Due to the relative dimensions of the second fluid chamber, the second fluid chamber 2 has merely the function of providing a reservoir for the first fluid chamber 1 and does not itself contribute much to the shape of the patient's breast 50. It is therefore accurate to say that the first fluid chamber 1 forms part of the breast implant 100, whereas the second fluid chamber 2 merely serving as a reservoir is “remotely implanted” and, thus, does not form part of the breast implant 100, even though it is located in the area of the patient's breast above the thorax. Alternatively, where the surface-to-volume ratio of the first and second fluid chambers 1, 2 are in the same order of magnitude, in particular almost identical, a volume change in either of the fluid chambers effectively contributes to a change in the shape of the patient's breast 50, in which case it is accurate to say that both the first and second fluid chambers 1, 2 form part of the breast implant 100.

It is conceivable and can even be preferable to place both the second fluid chamber 2 along with at least one first fluid chamber 1 underneath the patient's minor pectoralis muscle 40 or to place the first fluid chamber 1 between the patient's major and minor pectoralis muscles. In this case the first fluid chamber 1 would still be considered to form part of the breast implant 100 and the second fluid chamber 2 would still be considered implanted remote from the breast implant 100, when due to their substantially different surface-to-volume ratios the second fluid chamber 2 merely functions as a reservoir not substantially contributing to the overall shape of the patient's breast 50 and the first fluid chamber 1 mainly contributes to the shape variations of the patient's breast 50. Alternatively, when both the first and second fluid chambers 1, 2 have substantially the same surface-to-volume ratio, then also volume changes in the second fluid chamber 2 substantially contribute to the changes in the shape of the patient's breast 50 and, therefore, must be considered to form part of the breast implant 100.

FIGS. 476A and 476B show, in context with a thirteenth embodiment of a breast implant system, a different manner of how the second fluid chamber or chambers can be implanted remote from the breast implant under the pectoralis muscle. This embodiment differs from the one described in relation to FIGS. 475A and 475B in that the second fluid chamber 2 (or fluid chambers) is implanted between the patient's minor pectoralis muscle 40 and major pectoralis muscle 41. Also in this thirteenth embodiment, the second fluid chamber 2 is wide and flat as compared to the first fluid chamber 1, i.e. it has a substantially larger surface-to-volume ratio than the first fluid chamber 1, so that a substantial subjective volume change of the patient's breast 50 can be achieved by pumping fluid between the first and second fluid chambers 1, 2. The arrangement of the second fluid chamber 2 between the major and minor pectoralis muscles can be more convenient for the patient.

Again, it is conceivable and can even be preferable to place both the second fluid chamber 2 along with at least one first fluid chamber 1 between the pectoralis muscles.

### N: Treating Obesity and/or Reflux

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating devices for treating obesity and/or reflux, such as volume filling devices, stretching devices, and/or movement restriction devices. Examples of such devices for treating obesity and/or reflux will now be described.

Preferred embodiments will now be described in detail with reference to the drawing figures.

FIG. 477 shows a human patient 1, who is being treated for obesity. A volume filling device 100 is provided so that it reduces the inner volume of the stomach 12—the food cavity of the stomach, thereby affecting the patient's appetite. The function and the operation of this volume filling device will be described and explained in detail in the following description.

FIGS. 478 and 479 show in detail a first embodiment of an apparatus to treat obesity according to the invention, wherein FIG. 478 show a side view of the stomach while FIG. 479 is a sectional view taken along line IIb-IIb of FIG. 478. The apparatus comprises a volume filling device 100 implanted in a human patient. More specifically, in the embodiment of FIG. 478 the volume filling device 100 is invaginated in the wall 12a of the patient's stomach 12 on the outside of the stomach wall. The volume filling device may be at least substantially invaginated by a stomach wall portion of the patient by suturing a first part of a stomach wall serosa towards a second, different part of a stomach wall serosa, the serosa being the outer layer of the stomach wall. The volume filling device has a volume of less than 0.0002 m³ (200 ml) to allow invagination without penetrating the hepatogastric ligament. The body of the volume filling device 100 is elongated and shaped to rest against the wall 12a of the stomach 12 and further has an outer surface suitable to rest against this wall.

By invaginating the volume filling device 100 in part of the stomach wall, the size of the food cavity, generally designated 12b in FIG. 479, will be reduced, resulting in a more rapid feeling of satiety after food intake.

The volume filling device 100 preferably comprises an elastic or flexible material, such as silicone. In this way, the volume filling device can adapt to the movements of the stomach, the degree of food intake etc.

By providing the volume filling device from a bio-compatible material, the risk of the patient's body rejecting the implant is to a very large extent reduced.

The volume filling device 100 can be fixed to the wall 12a of the stomach 12 in a number of different ways. In the embodiment shown in FIG. 479, the volume filling device 100 is invaginated in the stomach wall 12a. After invagination, a number of stomach-to-stomach sutures or staples 14 are applied to keep the invagination in the short term. This allows growth of human tissue, keeping the invagination in the long term.

The volume filling device 100 preferably has an essentially round shape to not damage the stomach wall. An example thereof is shown in FIG. 480a, wherein the volume filling device is essentially egg-shaped. In another preferred embodiment, the volume filling device is slightly bent, such as the embodiment shown in FIG. 480b. However, since the stomach wall is strong many different shapes, forms, and dimensions may be used. In one embodiment, the volume filling device has a diameter of about 40 millimeters and a length of about 120 millimeters, resulting in a volume that is about half the volume of the patient's stomach. However, it is preferred that the maximum circumference of the volume filling device is at least 30 millimeters, more preferably at least 80 millimeters, and even more preferably at least 120 millimeters.

It is not necessary that the volume filling device is elongated. In the embodiment shown in FIG. 480c, the volume filling device 100 is essentially spherical or ball-shaped. In order to fill out the stomach, two or more such volume filling devices may be combined to achieve the desired decrease of the food cavity of the patient's stomach.

It has been mentioned that the volume filling device is secured by the stomach-to-stomach sutures or staples. In order to further improve the fixation, the volume filling device may be provided with a waist portion having smaller diameter that the maximum diameter of the volume filling device. Such volume filling device having a waist portion 10a is shown in FIG. 480d.

The volume filling device 100 may consist of at least two interconnectable portions so that each portion is easier to insert into the stomach and further through a hole in the stomach wall. Thus, FIG. 480e shows a volume filling device comprising two more or less spherical sub-parts 10b, 10c interconnected by a portion with which preferably has smaller diameter. The portion with smaller diameter may comprise an interconnection means with a reversible function allowing subsequent disconnection of the two interconnected sub-parts 10b, 10c. Such means may comprise a bayonet socket, a screw connection or the like, designated 10d in the figure. Alternatively, the portion with smaller diameter may comprise a fixed interconnection, such as resilient locking hooks provided on one of the sub-parts 10b, 10c and engaging the rim of a hole provided in the other one of the sub-parts 10b, 10c.

The configuration of the volume filling device 100 is not limited to one waist portion 10a. Thus, in FIG. 480f a volume filling device with two waist portions is shown.

In order to facilitate positioning of the volume filling device, an attachment means in the form of a handle or the like may be provided on the outer surface of the volume filling device. One example thereof is shown in FIG. 480g. wherein also a detail view of a handle 10e is shown. In a preferred embodiment, the attachment means is provided at an end portion of the volume filling device 100. In order to avoid protruding portion on the surface of the volume filling device 100, the handle 10e is provided flush with the outer surface of the volume filling device 100 and a recess 10f is arranged to allow a gripping tool or instrument (not shown in FIG. 480g) to achieve firm gripping around the handle 10e.

The volume filling device may comprise a tube for filling or emptying the volume filling device of a fluid or gel. By injecting fluid or gel into the volume filling device 100, the volume filling device is inflated to an inflated state, as will be described below. The size of the volume filling device can also be adjusted by moving fluid or gel therefrom to a different reservoir.

A volume filling device 100 adapted for this is shown in FIG. 480h. A tube 10g is fixedly attached to the volume filling device. This tube can be attached to a suitable instrument (not shown) or an injection port, which will be explained in detail below.

Instead of having a fixedly attached tube, the volume filling device 100 may comprise an inlet port 10h adapted for connection of a separate tube, see FIG. 480i.

It is important that the implanted volume filling device is firmly kept in place in the stomach wall in which it is invaginated. To this end, the volume filling device can be provided with one or more through holes adapted for receiving sutures or staples used for fixation of the invagination. Such an embodiment is shown in FIG. 480j, where the volume filling device 100 is provided with a row of holes 10i provided on a protruding flange-like protrusion on the volume filling device. In this embodiment, the row of holes extends along the longitudinal axis of the volume filling device.

FIG. 480k illustrates how sutures 14 are provided so that they run through the stomach wall 12a and through the holes 10i. In this way, the volume filling device is fixed in place in the pouch created from the stomach wall and will thus be prevented from sliding.

Although a plurality of holes is illustrated in the FIG. 480j, it will be appreciated that one single hole is sufficient to obtain improved fixation of the volume filling device 100.

FIG. 480m illustrates a volume filling device provided with an inlet port 10h. The volume filling device is invaginated in the stomach wall and the inlet port 10h is available for connection to a tube or the like from the abdominal area of the patient.

FIG. 480n illustrates an invaginated volume filling device wherein, instead of an inlet port, a fixed tube 10g extends into the abdominal area of the patient.

FIG. 480p is a figure similar to FIG. 480m but also illustrating tunneling of a connection tube 10g in the stomach wall between the inlet port 10h and the volume filling device 100.

It has been shown that the shape of the volume filling device can take many different forms. It will be appreciated that also the material of the volume filling device can vary. It is preferred that the volume filling device is provided with a coating in the form of a layer, such as a Parylene, polytetrafluoroethylene (PTFE), or polyurethane layer, or a combination of such layers, i.e., a multi-layer coating. This layer or multi-layer coating improves the properties of the volume filling device, such as its resistance to wear.

In one embodiment, the volume filling device comprises an inflatable device expandable to an expanded state. In this case, the inflatable device is provided with an inlet port for a fluid and is adapted to be connected to a gastroscopic instrument. This embodiment will now be described in detail with reference to FIGS. 481a-481d.

An inflatable volume filling device in its non-expanded state is shown in FIG. 481a. It is essentially a balloon-like, deflated device 100 having an inlet port 10h. In this state, the inflatable device has a diameter of a few millimeters at the most, allowing it to be inserted into the stomach through the esophagus of the patient by means of a gastroscopic, tube-like instrument 500, depicted in FIG. 4b. The instrument comprises an outer sleeve 500a and an inner sleeve 500b which can be displaced longitudinally relatively to the outer sleeve. The inner sleeve is provided with a cutter in the form of a cutting edge 615 at the distal end thereof. This cutting edge can be used for cutting a hole in the stomach wall, as will be explained in detail in the following.

When the instrument reaches a stomach wall, see FIG. 481c, the inner sleeve is brought forward from its position in the outer sleeve and into contact with the stomach wall 12a. The cutting edge 615 of the inner sleeve then cuts a hole in the stomach wall so as to allow subsequent insertion of the volume filling device 100 into and through this hole, see FIG. 481d. In order to push the volume filling device through the hole, a piston 502 may be provided in the instrument. Thus, the instrument further comprises a piston 502 adapted for pushing a deflated volume filling device 100 out from a position in the inner sleeve, this position being shown in FIG. 481b, to a position outside of the inner sleeve, this being shown in FIG. 481d.

In order to protect the deflated volume filling device 100 from the cutting edge 615 of the inner sleeve, a further protective sleeve (not shown) can be provided around the volume filling device.

An intraluminar method of invaginating a volume filling device 100 on the outside of the stomach wall 12a will now be described with reference to FIGS. 482a-482i. Initially, an instrument 600, preferably a gastroscopic instrument, is inserted into the mouth of the patient, see FIG. 482a. The instrument comprises an injection device 601, 602 for injecting either fluid or a device into the stomach of the patient. The instrument 600 further comprises a control unit 606 adapted for controlling the operation of the instrument. To this end, the control unit 606 comprises one or more steering devices, in the embodiment shown in the figure in the form of two joysticks 603 and two control buttons 604. A display 605 is provided for displaying the image provided by an optical device for viewing inside the stomach, such as a camera (not shown) arranged at the outer end of the elongated member 607, see FIGS. 482e-482i. The camera, which may comprise connecting electrical wires extending along the elongated member, may be assisted by a light source (not shown) placed distally on the elongated member for illuminating the inside of the stomach. The optical device may also comprise optical fibers placed along the elongated member and leading out from the patient's body for external viewing of the inside of the stomach.

The instrument is further inserted into the esophagus and into the stomach of the patient, see FIG. 482b. By means of the instrument 600, a hole 12b is created in the wall of the stomach 12. To this end, the instrument is provided with one or more cutters at the distal end thereof, for example in the way described above with reference to FIGS. 481a-481d. These cutters can of course be designed in different ways, such as a toothed drum cutter rotating about the center axis of the tube-like instrument.

After cutting a hole in the stomach wall, the distal end of the instrument 600 is inserted into and through the hole 12b so that it ends up outside the stomach wall 12a. This is shown in FIG. 482c, showing a side view of the stomach 12, and FIG. 482d, which is a sectional view through the stomach of FIG. 482c taken along the lines Vd-Vd.

The instrument 600 is adapted to create a “pocket” or “pouch” on the outside of the stomach 12 around the hole 12b in the stomach wall. Such an instrument and the method of providing the pouch will now be described.

FIGS. 482e-482i show a gastroscopic or laparoscopic instrument for invaginating a volume filling device 100 in the stomach wall 12a of the patient by creating a pouch of stomach wall 12a material in which the volume filling device is placed. The instrument, generally designated 600, and which may comprise the features described above with reference to FIGS. 481a-481d, comprises an elongated member 607 having a proximal end and a distal end, the elongated member 607 having a diameter less than that of the patient's esophagus and being flexible such as to allow introduction of the flexible elongated member 607 with its distal end first through the patient's throat, esophagus and into the stomach 12 to the stomach wall 12a.

The stomach penetration device or cutter 615 is provided on the elongated member 607 at the distal end thereof for penetrating the stomach wall 12a so as to create a hole in the stomach wall 12a, to allow introduction of the elongated member 607 through the hole. The stomach penetration device 615 could be adapted to be operable for retracting said stomach penetration device 615 after the stomach fundus wall 12a has been penetrated, for not further damaging tissue within the body. The instrument further comprises a special holding device 609 provided on the elongated member 607 on the proximal side to the penetration device 615.

The elongated member further comprises an expandable member 611 which is adapted to be expanded after the elongated member has penetrated the stomach wall 12a and thereby assist in the creation of a cavity or pouch adapted to hold the volume filling device 100. The expandable member 611 may comprise an inflatable circular balloon provided circumferentially around the distal end portion of the flexible elongated member 607.

The method steps when invaginating the volume filling device will now be described in detail.

After the instrument 600 has been inserted into the stomach 12, the stomach penetration device 615 is placed into contact with the stomach wall 12a, see FIG. 482e. The stomach penetration device or cutter 615 is then brought to create the hole 12b in the stomach wall, whereafter at least the expandable member 611 is brought through the hole 12b in the stomach wall. The special holding device 609 is in this step brought to a holding state wherein it expands radially so as to form an essentially circular abutment surface to the stomach wall 12a, see FIG. 482f. In this way, the insertion of the stomach penetration device 615 and the expandable member 611 through the hole 12b in the stomach wall is limited to the position shown in FIG. 482f.

The expandable member 611 is then expanded. In the case the expandable member comprises a balloon or the like, air or other fluid is injected into it.

The part of the elongated member 607 comprising the expandable member 611 is then retracted in the proximal direction, as indicated by the arrow in FIG. 482g, thereby pulling the stomach wall 12a into a basket or cup like structure created by the special holding device 609.

A suturing or stapling device 608 is further provided, either as a device connected to the elongated member 607 or as a separate instrument. The suturing or stapling member comprises a suturing or stapling end 613 which is adapted to close the cavity or pouch by means of stomach-to-stomach sutures or staples 14.

In a further step, illustrated in FIG. 482h, an inflatable volume filling device 100 is placed in its deflated state in the cup like structure. The volume filling device 100 is then inflated to its inflated or expanded state, see FIG. 482i. This inflation of the volume filling device 100 can be accomplished by injecting a fluid or a gel into the deflated volume filling device. It can also be accomplished by injecting a material which is allowed to cure, thereby forming a solid device 100. Thus, the volume filling device 100 shown in FIGS. 482h and 482i can illustrate either a balloon-like device which is subsequently filled with fluid or gel or alternatively a material which is simply injected into the cup like structure formed by the stomach wall 12a.

The fluid which is used to fill the volume filling device 100 could be any suitable fluid suitable to fill the inflatable device 100, such as a salt solution. In another embodiment, when this fluid is a fluid which is adapted to be transformed into solid state, the fluid could be liquid polyurethane.

In order to minimize or entirely eliminate leakage, the fluid is iso-tonic, i.e., it has the same osmolarity as human body fluids. Another way of preventing diffusion is to provide a fluid which comprises large molecules, such as iodine molecules.

The stomach-to-stomach sutures or staples are preferably provided with fixation portions exhibiting a structure, such as a net like structure, adapted to be in contact with the stomach wall to promote growth in of human tissue to secure the long-term placement of the volume filling device attached to the stomach wall.

After the inflatable device 100 has been inflated, partly or fully, the inlet port 10h (not shown in

FIGS. 482h and 482i) of the volume filling device 100, is sealed and the instrument 600 is retracted from the hole 12b, which is subsequently closed in some suitable way, such as by means of the instrument 600. The instrument is then removed from the stomach 12 and the inflatable device 100 in its inflated or expanded state is invaginated by a stomach wall portion of the patient on the outside of the stomach wall.

This reduces the inner volume of the stomach, thereby affecting the patent's appetite.

During one or more of the above described steps, the stomach may be inflated with gas, preferably by means of the gastroscopic instrument.

The volume filling device 100 described above with reference to FIGS. 482a-482i has been described as an inflatable volume filling device. It will be appreciated that is also can be an elastic volume filling device with an elasticity allowing compression so as to be inserted into a gastroscopic instrument and which expands to an expanded state after leaving the instrument.

An alternative embodiment of an apparatus for treating obesity will now be described with reference to FIGS. 483a and 483b, showing a sectional view of a stomach in which a volume filling device is invaginated in the stomach wall on the outside thereof together with a system for regulating the size of the volume filling device. The volume filling device is an inflatable device as described above with reference to FIGS. 482a-h and thus comprises a fluid. The inflatable device 100 thus forms a fluid chamber, in which fluid is allowed to flow. The inflatable device thereby forms an expandable chamber that can change the volume it occupies in the stomach wall, thereby forming a hydraulically or pneumatically regulated inflatable device.

In FIG. 483a, an injection port 16 for fluids is connected to the inflatable volume filling device 100 by means of a conduit 18 in the form of a tube. The inflatable device 100 is thereby adapted to be regulated, preferably non-invasively, by moving liquid or air from the injection port 16 to the chamber formed by the inflatable device. By using a hypodermic needle or the like, the amount of fluid in the inflatable device 100 can thus be adjusted, thereby adjusting the size of the adjustable device. The injection port 16 can also be used simply for refilling the volume filling device 100.

A similar embodiment is shown in FIG. 483b. However, in this embodiment the injection port 16 has been replaced by an adjustable regulation reservoir 17 in fluid connecting with the volume filling device 100 via a conduit 18. When the regulation reservoir 17 is pressed, the volume thereof decreases and hydraulic fluid is moved from the reservoir to the chamber formed by the inflatable device 100 via the conduit or tube 18, enlarging or expanding the inflatable device 100. In this way, the volume filling device is non-invasively adjustable postoperatively.

The regulation reservoir 17 can be regulated in several ways. In an alternative embodiment, the regulation reservoir 17 is regulated by manually pressing a regulation reservoir. In other words, the regulation reservoir is regulated by moving a wall of the reservoir. It is then preferred that the regulation reservoir is placed subcutaneously and non-invasive regulation is thereby achieved.

It will be appreciated that instead of hydraulic operation, pneumatic operation can be used, wherein air instead of hydraulic fluid is moved between the regulation reservoir and the chamber formed by the inflatable device 100. Preferable the regulation reservoir has a locking position to keep it in the desired position. If the patient compresses the reservoir it preferably stays compressed and releases after pressing again.

Any kind of hydraulic solution may be used for the inflatable device. The hydraulic solution may be driven by both mechanically and be powered with any motor or pump as well as manually.

In another embodiment, shown in FIG. 484, a motor 40 is adapted to move a wall of the regulation reservoir 17. The powered regulation reservoir 17 is then preferably placed in the abdomen of the patient. In this embodiment, a wireless external remote control unit forming part of an external energy transmission device 34 can be provided to perform non-invasive regulation of the motor via an energy transforming device 30, which is adapted to supply an energy consuming operation device, in the present example the motor 40, with energy via a power supply line 32.

The remote control may comprise a wireless energy transmitter, whereby the non-invasive regulation is performed by the energy transmitter. When the regulation is performed by means of a remote control an internal power source for powering the regulating device is provided. The internal energy source can for example be a chargeable implanted battery or a capacitor or a device for receiving wireless energy transmitted from outside the body of the patient. Different ways of regulating the inflatable device 100 will be described below with reference to FIGS. N22-N41.

In yet an alternative embodiment, shown in FIG. 8, the apparatus for treating obesity comprises a pump 44, wherein the reservoir is regulated by the pump 44 pumping fluid or air from the reservoir to the chamber formed by the inflatable device. Different configurations of this pump will be described below with reference to FIGS. N22-N41.

Yet an alternative embodiment of an apparatus for treating obesity will now be described with reference to FIG. 486, which shows a stomach 12 of a patient who is treated for obesity. This embodiment is similar to the one described above with reference to FIG. 484 and the apparatus comprises a volume filling device in the form of an inflatable device 100 which is invaginated in the wall 12a of the patient's stomach 12. However, in this case the invagination has been performed in the fundus, i.e., the upper portion of the stomach, where the number of receptors in the stomach wall is large, and the inflatable device functions as a stretching device for part of the stomach fundus wall.

A regulation reservoir 17 for fluids is connected to the inflatable device by means of a conduit 18 in the form of a tube. The inflatable device 100 is thereby adapted to be regulated, preferably non-invasively, by moving liquid or air from the regulation reservoir 17 to the chamber formed by the inflatable device 100. The regulation of the inflatable device 100 preferably comprises a reversed servo, i.e., a small volume is actuated for example by the patient's finger and this small volume is in connection with a larger volume, i.e., the regulation reservoir 17.

Thus, the inflatable device 100 is placed outside the stomach wall and is adapted to stretch a part of the stomach fundus wall, thereby affecting the patient's appetite. By enlarging the size of the stretching device, the stomach fundus wall 12a surrounding the inflatable stretching device 100 is stretched since the circumference of the inflatable stretching device 100 is increased. By this stretching, the receptors in the stomach wall indicate that the stomach is full, thereby creating a feeling of satiety to the patient. Correspondingly, when the stretching device 100 is contracted, the receptors indicate that the stomach is not full, thereby returning the feeling of hunger. It will be appreciated that this embodiment combines the effects of both reducing the volume of the stomach food cavity and stretching part of the stomach wall, thereby increasing the treatment effect.

The expansion and contraction of the stretching device 100 can be performed under direct control of the patient. Alternatively, the expansion and contraction can be performed according to a pre-programmed schedule.

In a preferred embodiment, shown in FIG. 487, a sensor 19 is provided at a suitable position, such as at the esophagus. The volume filling device 100 in the form of the inflatable stretching device is similar to the one shown in FIG. 486. By providing one or more sensors, the apparatus for treating obesity can be automated in that the size of the volume filling device 100 in the form of the inflatable stretching device is adjusted depending on the amount of food entering the food cavity of the stomach. The fluid is thereby moved between the inflatable volume filling device 100 and a fluid reservoir.

The apparatus for treating obesity can have the additional functionality of treating reflux. An embodiment having this function is shown in FIG. 488, wherein the volume filling device 100 is invaginated in the stomach wall close to and at least partially above the patient's cardia 26 when the patient is in a standing position and is fixed to a position above the cardia area 26 by a fixation, such as sutures or staples 14a. For example a direct or indirect fixation to the diaphragm muscle or associated muscles may be provided. As an alternative a direct or indirect fixation to the esophagus above and close to the angle of His can be provided. In this alternative embodiment, the volume filling device 100 rests in a position against stomach wall of the fundus when implanted and which also fills a volume above the cardia area 26 between the cardia and the diaphragm muscle so that the cardia is prevented from slipping up into the thorax cavity, whereby reflux disease is prevented.

Such a volume filling device 100 may be used for keeping electronics and/or an energy source and/or hydraulic fluid. Hydraulic fluid from that device may be distributed to several smaller inflatable device areas to vary the stretching area from time to time avoiding any possible more permanent stretching effect of the stomach wall. Even mechanically several stretching areas may be used.

In an alternative embodiment, which is shown in FIG. 489, the volume of an inflatable volume filling device 100 may be in fluid connection with one or more preferably smaller inflatable devices or chambers that functions as adjustable stretching devices 50. These chambers are adapted to communicate with fluid or air being moved between the chambers.

Thus, the large chamber 100 is adapted to, with its main volume to be a volume filling device for reducing the size of the food cavity and for treating reflux disease and the one or several small chambers are adapted to function as the inflatable devices to treat obesity, wherein the main chamber is adapted to communicate with fluid or air to the small chambers causing a stretching effect in the stomach wall thereby further treating obesity.

In FIGS. 490-493, different embodiments embodying a combination of a volume filling device invaginated in the central or lower portion of the stomach and a stretching device invaginated in the upper portion or fundus of the patient's stomach. Thus, in FIG. 490 there is shown an adjustable volume filling device 100, which is invaginated in the stomach wall of a patient's stomach 12. Additionally, an adjustable stretching device 50 with the previously described function is invaginated in the stomach fundus wall of the patient. It is preferred that the volume filling device 100 is substantially larger than the stretching device 50.

The volume filling device 100 and the stretching device 50 are in fluid communication with each other via a fluid communication device comprising a first fluid tube 52, in which a pump 54 is provided.

The pump 54 is under the control from an energy transforming device 30, which is adapted to supply the pump 54 with energy via a power supply line 56. The energy transforming device 30 is also connected to a sensor 19 provided in the esophagus of the patient so that food intake can be detected.

The volume filling device 100 and the stretching device 50 are also in fluid communication with each other via a second fluid tube 58, which preferably has a smaller cross-sectional area than the first fluid tube 52.

The operation of this arrangement is as follows. The volume filling device 100 functions as in the above described embodiments, i.e., it reduces the size of the food cavity of the patient's stomach 12.

Additionally, when the stretching device 50 is enlarged by pumping fluid from the volume filling device 100 and to the stretching device 50 by means of the pump 54, the stomach fundus wall is stretched, creating a feeling of satiety for the patient. Thus, for example when food intake is detected by means of the sensor 19, fluid is automatically pumped into the stretching device 50 to increase the feeling of satiety and thereby limit the food intake.

When fluid has been injected into the stretching device 50, the internal pressure therein is higher than the internal pressure in the volume filling device 100. This difference in pressure will create a flow of fluid in the second, preferably narrower tube 58 from the stretching device 50 to the volume filling device 100. The flow rate will be determined by among other things the difference in pressure and the cross-sectional area of the second tube 58. It is preferred that the second tube is so dimensioned, that the pressures in the volume filling device 100 and the stretching device 50 will return to equilibrium after 3 hours after fluid has been injected into the stretching device 50 to create the feeling of satiety.

In this embodiment, the function of the second tube 58 is to allow fluid to return from the stretching device 50 to the volume filling device 100. It will be appreciated that this function also can be performed by the pump 54 in the first tube 52 and that the second tube 58 then can be omitted.

FIG. 491 illustrates an embodiment similar to the one illustrated in FIG. 490. Thus, there is provided an adjustable volume filling device 100, which is invaginated in the stomach wall of a patient's stomach 12. Additionally, an adjustable stretching device 50 with the previously described function is invaginated in the stomach fundus wall of the patient. It is preferred that the volume filling device 100 is substantially larger than the stretching device 50.

The volume filling device 100 and the stretching device 50 are in fluid communication with each other via a first fluid tube 52, and a second fluid tube, which preferably has a smaller cross-sectional area than the first tube. However, instead of a pump, there is provided a non-return valve 60 in the first fluid tube 52 instead of an energized pump. This non-return valve 60 allows fluid to flow in the direction from the volume filling device 100 and to the stretching device 50 but not vice versa. This means that this embodiment may be entirely non-energized. Instead, it operates according to the following principles.

When the food cavity of the stomach 12 is essentially empty, there is a state of equilibrium between the internal pressure of the volume filling device 100 and the stretching device 50. In this state, the stretching device is in a non-stretch state, i.e., it does not stretch a part of the stomach fundus wall and thus does not create a feeling of satiety.

When the patient starts to eat, food will enter the food cavity of the stomach 12. This will create increased pressure on the stomach wall in which the volume filling device 100 is invaginated and the internal pressure therein will increase. Also, the stomach wall muscles will begin to process the food in the food cavity by contraction, which also contributes to an increased internal pressure in the volume filling device 100.

Since the internal pressure in the stretching device 50 will remain essentially unchanged, because it is located in the upper part of the stomach 12 where no food is exerting a pressure on the stomach wall, a fluid flow will be created through the first and second fluid tubes 52, 58 in the direction from the volume filling device 100 and to the stretching device 50. This in turn will increase the volume of the stretching device 50, which, by stretching the stomach fundus wall, will provide a feeling of satiety to the patient.

A fluid flow from the stretching device 50 to the volume filling device 100 through the second tube 58 will return the pressure of these devices to equilibrium as described above with reference to FIG. 490.

FIG. 492 illustrates an embodiment, which is similar to the one shown in FIG. 491 but with the addition of an injection port 16, which is used for refilling the fluid system comprising the volume filling device 100 and the stretching device 50 or alternatively for actively adjusting the size thereof.

Similarly, FIG. 493 illustrates an embodiment wherein the stretching device 50 can be actively regulated by manually pressing an adjustment reservoir which is provided subcutaneously below the patient's skin, similar to the embodiment shown in FIG. 486. Thus, a regulation reservoir 17 for fluids is connected to the inflatable device by means of a conduit 18 in the form of a tube. The stretching device 50 is thereby adapted to be regulated, non-invasively, by moving liquid or air from the regulation reservoir 17 to the chamber formed by the inflatable device. The regulation of the stretching device 50 preferably comprises a reversed servo, i.e., a small volume is actuated for example by the patient's finger and this small volume is in connection with a larger volume.

An alternative placement of the volume filling device 100 is shown in FIGS. 494 and 495, wherein FIG. 495 shows a sectional view through the stomach shown in FIG. 494 along the line XVIIb-XVIIb. There, the volume filling device 100 is adapted to be placed inside the wall of the stomach 12, such as via a gastroscope or similar intraluminar instrument, and resting against the inside of the stomach wall 12a. The inflatable device can be kept invaginated by means of sutures or staples 14, like in the embodiment of FIGS. 478 and 2b. In this embodiment, no hole is required in the stomach wall. Instead, a method of providing the volume filling device 100 can comprise the following steps, which will be explained with reference to FIGS. 496a-496i showing an invagination instrument

The invagination instrument, generally designated 630, comprises an elongated tube member 632 similar to the elongated member 607 described above with reference to FIGS. 482a-482i. Thus, it can be connected to a control unit 606, see FIG. 482a. The invagination instrument 630 further comprises a perforated suction portion 634, which preferably is elongated. The suction portion 634 exhibits a plurality of small holes 636, into which air will be sucked by providing suction in the tube member 632.

This suction effect will be used to create a “pocket” or “pouch” in a part of a stomach wall, generally designated 12a.

In other words, when the tip of the suction portion 634 is pressed against the stomach wall 12a, see FIG. 496a, a small recess will be formed therein. When the suction portion 634 is further pressed against the stomach wall 12a, see FIG. 496b, a larger recess will be formed. The part of the stomach wall 12a that forms the recess will, due to the suction effect, adhere to the suction portion 634 of the invagination instrument 630. As the suction portion 634 is further pressed into the stomach wall 12a, see

FIG. 496c, a deeper recess will be formed until the entire suction portion 634 is embedded in the recess, see FIG. 496d.

The rim of the recess will at this stage be fixated by means of fixation elements 638 and the suction portion be removed from the instrument, see FIG. 496e. A compressed elastic volume filling device 100 will subsequently be inserted into the recess, see FIG. 496f, for example in the way described above with reference to FIG. 481d. This compressed volume filling device is then expanded to its final shape, see FIG. 496g, where after the pouch is sealed by suturing or stapling by means of the fixation elements, see FIG. 496h.

All the alternatives described above with reference to FIGS. 478-493 are also applicable to the embodiment described with reference to FIGS. 494, 495 and 496, i.e., to the embodiment where the volume filling device is invaginated on the inside of the stomach wall.

FIGS. 497a-497j show an instrument for use in a method of engaging a volume filling device 100 to the stomach wall 12 of a patient. The instrument is adapted to be inserted through a narrow tube shaped object such as a gastroscope, used in an intraluminar procedure, or a laparoscopic trocar used in a laparoscopic procedure. The instrument comprises an elongated member 650 which is adapted to be flexible by means of a construction comprising multiple ring shaped members, however it is equally conceivable that said elongated member 650 is adapted to be flexible by means of said elongated member 650 being made of a flexible or adjustable material. The elongated member 650 is inserted into the body and placed in proximity to the stomach wall 12a of the patient, from the outside or inside thereof. The elongated member 650 has a special holding device 651 adapted to hold the stomach by means of mechanical grabbing members or vacuum. The special holding device 651 comprises a first joint 652 and a second joint 653, which enable the special holding device 651 be operable in relation to the elongated member 650 and thereby place the part of the special holding device comprising the mechanical grabbing members or vacuum elements into contact with the stomach wall 12a of the patient. FIG. 497b shows the special holding device 651 when placed in contact with the stomach wall 12a of the human patient, after which the special holding device 651 connects to the stomach wall 12, for holding the stomach wall 12a.

FIG. 497c shows the instrument when the step of advancing a pushing rod 654 from the elongated member 650 is performed. The pushing rod 654 pushes the stomach wall 12 to create a cavity or pouch thereof. FIG. 497d shows the instrument turned 90° in relation to FIGS. 497a-c. This view shows the special holding devices 651a,b operably attached to two sides of the elongated member 650 and being in contact with the stomach wall 12a, holding the stomach wall 12 as the pushing rod 654 pushes to create a cavity or pouch. When the pushing rod 654 has pushed the stomach wall 12 to a desired position the special holding devices 651a,b moves towards the pushing rod 654 and thereby closes the cavity or pouch.

After the cavity or pouch has been created it needs to be sealed. FIG. 497f shows the advancement of a suturing or stapling device 655 from the elongated member 650. The suturing or stapling device 655 is positioned in connection with the stomach wall after which the suturing or stapling device commences with the suturing or stapling of the stomach wall 12a, creating a seal of stomach-to-stomach sutures or staples 14. The instrument is moved along the stomach wall 12a of the patient and thereby a cavity or pouch is created and sealed using the instrument, as shown in FIGS. 497g and 497h.

When a cavity or pouch or desired size has been created and sealed an inserting member 656 is advanced from the elongated member 650. The inserting member 656 is adapted to insert a volume filling device 100 being inflatable, as described earlier in this application. After the inserting member 656 has been positioned in the cavity or pouch the volume filling device 100 is inserted through the inserting member 656 and into the cavity or pouch by means of a pressurized fluid or gas, or a mechanical advancement member pushing said inflatable volume filling device 100 into the cavity or pouch. The insertion member then inflates the inflatable volume filling device with a fluid or gas and seals of the final section of the pouch using stomach to stomach sutures or staples 14. The embodiment described explains the process of inserting an inflatable volume filling device, however it is equally conceivable that the volume filling device 100 is expandable by means of the volume filling device 100 being made of an elastic material.

FIGS. 498a-498f show an instrument for use in a method of engaging a volume filling device 100 to the stomach wall 12a of a patient. The instrument is adapted to be inserted through a narrow tube shaped object such as a gastroscope, used in an intraluminar procedure, or a laparoscopic trocar used in a laparoscopic procedure. The instrument comprises an elongated member 660 which is adapted to be flexible by means of a construction comprising multiple ring shaped members, however it is equally conceivable that said elongated member 660 is adapted to be flexible by means of said elongated member 660 being made of a flexible or adjustable material. The elongated member 660 is inserted into the body and placed in proximity to the stomach wall 12a of the patient, from the outside or inside thereof. The elongated member 660 has multiple special holding devices 661 adapted to hold the stomach by means of mechanical grabbing members or vacuum. The special holding devices 661 are locked in a position alongside the elongated member 660 by means of a locking ring 662. The special holding devices are made of a flexible material end pre-bent to expand into a funnel-shaped device when said locking ring 662 is removed. The special holding device in its funnel shaped expandable state is shown in FIG. 498b.

FIG. 498b further shows the special holding device 661 when placed in contact with the stomach wall 12a of the human patient, after which the special holding device 661 connects to the stomach wall 12a, for holding the stomach wall 12a. FIG. 498c shows the instrument when the step of advancing a pushing rod 664 from the elongated member 660 is performed. The pushing rod 664 pushes the stomach wall 12a to create a cavity or pouch thereof. When the pushing rod 664 has pushed the stomach wall 12a to a desired position the special holding devices 661 moves towards the pushing rod 664 and thereby closes the cavity or pouch.

After the cavity or pouch has been created it needs to be sealed. FIG. 498d shows the advancement of a suturing or stapling device 665 from the elongated member 660. The suturing or stapling device 665 is positioned in connection with the stomach wall 12 after which the suturing or stapling device 665 commences with the suturing or stapling of the stomach wall 12a, creating a seal of stomach-to-stomach sutures or staples 14. Thereafter an inserting member 656 is advanced from the elongated member 660 and the special holding devices 661 are retracted. The inserting member 656 is adapted to insert a volume filling device 100 being inflatable, as described earlier in this application.

After the inserting member 656 has been positioned in the cavity or pouch the volume filling device 100 is inserted through the inserting member 656 and into the cavity or pouch by means of a pressurized fluid or gas, or a mechanical advancement member pushing said inflatable volume filling device 100 into the cavity or pouch. The inserting member 656 then inflates the inflatable volume filling device with a fluid or gas and seals of the final section of the pouch using stomach to stomach sutures or staples 14. The embodiment described explains the process of inserting an inflatable volume filling device 100, however it is equally conceivable that the volume filling device 100 is expandable by means of the volume filling device 100 being made of an elastic material. FIG. 498f shows the volume filling device 100 as the volume filling device 100 is invaginated in the stomach wall 12a, in a cavity or pouch sealed with stomach-to-stomach sutures or staples 14.

FIG. 499a shows an instrument used in a method of engaging the volume filling device according to any of the embodiments of the application to the stomach wall 12a. The instrument comprises an elongated member 670 which is adapted to be flexible by means of a construction comprising multiple ring shaped members, however it is equally conceivable that said elongated member 670 is adapted to be flexible by means of said elongated member 670 being made of a flexible or adjustable material. The elongated member 670 is inserted into the body and placed in proximity to the stomach wall 12a of the patient, from the inside thereof. A stomach penetrating member 672 is placed in the distal end of the elongated member 670, retractably fixated to a protective sleeve 673 adapted to protect the tissue of the body from the sharp penetrating member 672 or cutter 672 after the cutting operation has been performed.

FIG. 499b shows the instrument comprising the elongated member 670 after the cutting operation has been performed and the stomach penetrating member or cutter 672 has been retracted into the protective sleeve 673. A guiding wire 671 is pushed through the elongated member 670, through the hole made in the stomach wall 12a and out through the abdomen and placed on the inside of the patient's skin, which is penetrated from the outside to enable the guiding wire 671 to exit the abdomen. The guiding wire 671 can then be used to guide a conduit 18 or a lead attached to the volume filling device 100 being placed in the stomach from the inside thereof. The volume filling device 100 with the conduit 18 or electrical lead being a volume filling device 100 according to any of the embodiments of this application.

The guiding of the conduit 18 or electrical lead enables the attachment of the conduit 18 or electrical lead to a control unit (not shown) placed subcutaneously in the patient from the outside of the abdomen.

FIGS. 498a-498f show an instrument for use in a method of engaging a volume filling device 100 to the stomach wall 12a of a patient. The instrument is adapted to be inserted through a narrow tube shaped object such as a gastroscope, used in an intraluminar procedure, or a laparoscopic trocar used in a laparoscopic procedure. The instrument comprises an elongated member 660 which is adapted to be flexible by means of a construction comprising multiple ring shaped members, however it is equally conceivable that said elongated member 660 is adapted to be flexible by means of said elongated member 660 being made of a flexible or adjustable material. The elongated member 660 is inserted into the body and placed in proximity to the stomach wall 12a of the patient, from the outside or inside thereof. The elongated member 660 has multiple special holding devices 661 adapted to hold the stomach by means of mechanical grabbing members or vacuum. The special holding devices 661 are locked in a position alongside the elongated member 660 by means of a locking ring 662. The special holding devices are made of a flexible material end pre-bent to expand into a funnel-shaped device when said locking ring 662 is removed. The special holding device in its funnel shaped expandable state is shown in FIG. 498b.

FIG. 498b further shows the special holding device 661 when placed in contact with the stomach wall 12a of the human patient, after which the special holding device 661 connects to the stomach wall 12a, for holding the stomach wall 12a. FIG. 498c shows the instrument when the step of advancing a pushing rod 664 from the elongated member 660 is performed. The pushing rod 664 pushes the stomach wall 12a to create a cavity or pouch thereof. When the pushing rod 664 has pushed the stomach wall 12a to a desired position the special holding devices 661 moves towards the pushing rod 664 and thereby closes the cavity or pouch.

After the cavity or pouch has been created it needs to be sealed. FIG. 498d shows the advancement of a suturing or stapling device 665 from the elongated member 660. The suturing or stapling device 665 is positioned in connection with the stomach wall 12a after which the suturing or stapling device 665 commences with the suturing or stapling of the stomach wall 12a, creating a seal of stomach-to-stomach sutures or staples 14. Thereafter an inserting member 656 is advanced from the elongated member 660 and the special holding devices 661 are retracted. The inserting member 656 is adapted to insert a volume filling device 100 being inflatable, as described earlier in this application.

After the inserting member 656 has been positioned in the cavity or pouch the volume filling device 100 is inserted through the inserting member 656 and into the cavity or pouch by means of a pressurized fluid or gas, or a mechanical advancement member pushing said inflatable volume filling device 100 into the cavity or pouch. The inserting member 656 then inflates the inflatable volume filling device with a fluid or gas and seals of the final section of the pouch using stomach to stomach sutures or staples 14. The embodiment described explains the process of inserting an inflatable volume filling device 100, however it is equally conceivable that the volume filling device 100 is expandable by means of the volume filling device 100 being made of an elastic material. FIG. 20 f shows the volume filling device 100 as the volume filling device 100 is invaginated in the stomach wall 12a, in a cavity or pouch sealed with stomach to stomach sutures or staples 14.

FIG. 499a shows an instrument used in a method of engaging the volume filling device according to any of the embodiments of the application to the stomach wall 12a. The instrument comprises an elongated member 670 which is adapted to be flexible by means of a construction comprising multiple ring shaped members, however it is equally conceivable that said elongated member 670 is adapted to be flexible by means of said elongated member 670 being made of a flexible or adjustable material. The elongated member 670 is inserted into the body and placed in proximity to the stomach wall 12a of the patient, from the inside thereof. A stomach penetrating member 672 is placed in the distal end of the elongated member 670, retractably fixated to a protective sleeve 673 adapted to protect the tissue of the body from the sharp penetrating member 672 or cutter 672 after the cutting operation has been performed.

FIG. 499b shows the instrument comprising the elongated member 670 after the cutting operation has been performed and the stomach penetrating member or cutter 672 has been retracted into the protective sleeve 673. A guiding wire 671 is pushed through the elongated member 670, through the hole made in the stomach wall 12a and out through the abdomen and placed on the inside of the patient's skin, which is penetrated from the outside to enable the guiding wire 671 to exit the abdomen. The guiding wire 671 can then be used to guide a conduit 18 or a lead attached to the volume filling device 100 being placed in the stomach from the inside thereof. The volume filling device 100 with the conduit 18 or electrical lead being a volume filling device 100 according to any of the embodiments of this application.

The guiding of the conduit 18 or electrical lead enables the attachment of the conduit 18 or electrical lead to a control unit 42 placed subcutaneously in the patient from the outside of the abdomen.

FIG. 666 illustrates a system for treating a disease comprising an apparatus 100 comprising a volume filling device of the present invention placed in the abdomen of a patient. An implanted energy-transforming device 1002 is adapted to supply energy consuming components of the apparatus with energy via a power supply line 1003. An external energy-transmission device 200 for non-invasively energizing the apparatus 100 transmits energy by at least one wireless energy signal. The implanted energy-transforming device 1002 transforms energy from the wireless energy signal into electric energy which is supplied via the power supply line 1003.

The implanted energy-transforming device 1002 may also comprise other components, such as: a coil for reception and/or transmission of signals and energy, an antenna for reception and/or transmission of signals, a microcontroller, a charge control unit, optionally comprising an energy storage, such as a capacitor, one or more sensors, such as temperature sensor, pressure sensor, position sensor, motion sensor etc., a transceiver, a motor, optionally including a motor controller, a pump, and other parts for controlling the operation of a medical implant.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field.

The wireless energy-transmission device 200 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal.

Generally speaking, the energy-transforming device 1002 is provided for transforming wireless energy of a first form transmitted by the energy-transmission device 200 into energy of a second form, which typically is different from the energy of the first form. The implanted apparatus 100 is operable in response to the energy of the second form. The energy-transforming device 1002 may directly power the apparatus with the second form energy, as the energy-transforming device 1002 transforms the first form energy transmitted by the energy-transmission device 200 into the second form energy. The system may further include an implantable accumulator, wherein the second form energy is used at least partly to charge the accumulator.

Alternatively, the wireless energy transmitted by the energy-transmission device 200 may be used to directly power the apparatus, as the wireless energy is being transmitted by the energy-transmission device 200. Where the system comprises an operation device for operating the apparatus, as will be described below, the wireless energy transmitted by the energy-transmission device 200 may be used to directly power the operation device to create kinetic energy for the operation of the apparatus.

The wireless energy of the first form may comprise sound waves and the energy-transforming device 1002 may include a piezo-electric element for transforming the sound waves into electric energy.

The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus.

Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus.

The external energy-transmission device 200 also includes a wireless remote control having an external signal transmitter for transmitting a wireless control signal for non-invasively controlling the apparatus. The control signal is received by an implanted signal receiver which may be incorporated in the implanted energy-transforming device 1002 or be separate there from.

The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field.

The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals.

FIG. 667 illustrates the system of FIG. 666 in the form of a more generalized block diagram showing the apparatus 100, the energy-transforming device 1002 powering the apparatus 100 via power supply line 1003, and the external energy-transmission device 200, The patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 1006 operable for example by polarized energy also is implanted in the patient for reversing the apparatus 100. When the switch is operated by polarized energy the wireless remote control of the external energy-transmission device 200 transmits a wireless signal that carries polarized energy and the implanted energy-transforming device 1002 transforms the wireless polarized energy into a polarized current for operating the electric switch 1006. When the polarity of the current is shifted by the implanted energy-transforming device 1002 the electric switch 1006 reverses the function performed by the apparatus 100.

FIG. 669 shows an embodiment identical to that of FIG. 667, except that an operation device 1007 implanted in the patient for operating the apparatus 100 is provided between the implanted energy-transforming device 1002 and the apparatus 100. This operation device can be in the form of a motor 1007, such as an electric servomotor. The motor 1007 is powered with energy from the implanted energy-transforming device 1002, as the remote control of the external energy-transmission device 200 transmits a wireless signal to the receiver of the implanted energy-transforming device 1002.

FIG. 670 shows an embodiment identical to that of FIG. 667, except that it also comprises an operation device is in the form of an assembly 1008 including a motor/pump unit 1009 and a fluid reservoir 1010 is implanted in the patient. In this case the apparatus 100 is hydraulically operated, i.e. hydraulic fluid is pumped by the motor/pump unit 1009 from the fluid reservoir 1010 through a conduit 1011 to the apparatus 100 to operate the apparatus, and hydraulic fluid is pumped by the motor/pump unit 1009 back from the apparatus 100 to the fluid reservoir 1010 to return the apparatus to a starting position. The implanted energy-transforming device 1002 transforms wireless energy into a current, for example a polarized current, for powering the motor/pump unit 1009 via an electric power supply line 1012.

Instead of a hydraulically operated apparatus 100, it is also envisaged that the operation device comprises a pneumatic operation device. In this case, the hydraulic fluid can be pressurized air to be used for regulation and the fluid reservoir is replaced by an air chamber.

In all of these embodiments the energy-transforming device 1002 may include a rechargeable accumulator like a battery or a capacitor to be charged by the wireless energy and supplies energy for any energy consuming part of the system.

As an alternative, the wireless remote control described above may be replaced by manual control of any implanted part to make contact with by the patient's hand most likely indirect, for example a press button placed under the skin.

FIG. 671 shows an embodiment comprising the external energy-transmission device 200 with its wireless remote control, the apparatus 100, in this case hydraulically operated, and the implanted energy-transforming device 1002, and further comprising a hydraulic fluid reservoir 1013, a motor/pump unit 1009 and an reversing device in the form of a hydraulic valve shifting device 1014, all implanted in the patient. Of course the hydraulic operation could easily be performed by just changing the pumping direction and the hydraulic valve may therefore be omitted. The remote control may be a device separated from the external energy-transmission device or included in the same. The motor of the motor/pump unit 1009 is an electric motor. In response to a control signal from the wireless remote control of the external energy-transmission device 200, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 100. The remote control of the external energy-transmission device 200 controls the hydraulic valve shifting device 1014 to shift the hydraulic fluid flow direction between one direction in which the fluid is pumped by the motor/pump unit 1009 from the hydraulic fluid reservoir 1013 to the apparatus 100 to operate the apparatus, and another opposite direction in which the fluid is pumped by the motor/pump unit 1009 back from the apparatus 100 to the hydraulic fluid reservoir 1013 to return the apparatus to a starting position.

FIG. 672 shows an embodiment comprising the external energy-transmission device 200 with its wireless remote control, the apparatus 100, the implanted energy-transforming device 1002, an implanted internal control unit 102 controlled by the wireless remote control of the external energy-transmission device 200, an implanted accumulator 1016 and an implanted capacitor 1017. The internal control unit 102 arranges storage of electric energy received from the implanted energy-transforming device 1002 in the accumulator 1016, which supplies energy to the apparatus 100. In response to a control signal from the wireless remote control of the external energy-transmission device 200, the internal control unit 102 either releases electric energy from the accumulator 1016 and transfers the released energy via power lines 1018 and 1019, or directly transfers electric energy from the implanted energy-transforming device 1002 via a power line 1020, the capacitor 1017, which stabilizes the electric current, a power line 1021 and the power line 1019, for the operation of the apparatus 100.

The internal control unit is preferably programmable from outside the patient's body. In a preferred embodiment, the internal control unit is programmed to regulate the apparatus 100 according to a pre-programmed time-schedule or to input from any sensor sensing any possible physical parameter of the patient or any functional parameter of the system.

In accordance with an alternative, the capacitor 1017 in the embodiment of FIG. 672 may be omitted. In accordance with another alternative, the accumulator 1016 in this embodiment may be omitted.

FIG. 673 shows an embodiment identical to that of FIG. 667, except that a battery 1022 for supplying energy for the operation of the apparatus 100 and an electric switch 1023 for switching the operation of the apparatus 100 also are implanted in the patient. The electric switch 1023 may be controlled by the remote control and may also be operated by the energy supplied by the implanted energy-transforming device 1002 to switch from an off mode, in which the battery 1022 is not in use, to an on mode, in which the battery 1022 supplies energy for the operation of the apparatus 100.

FIG. 674 shows an embodiment identical to that of FIG. 673, except that an internal control unit 102 controllable by the wireless remote control of the external energy-transmission device 200 also is implanted in the patient. In this case, the electric switch 1023 is operated by the energy supplied by the implanted energy-transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the apparatus 100.

FIG. 675 shows an embodiment identical to that of FIG. 674, except that an accumulator 1016 is substituted for the battery 1022 and the implanted components are interconnected differently. In this case, the accumulator 1016 stores energy from the implanted energy-transforming device 1002. In response to a control signal from the wireless remote control of the external energy-transmission device 200, the internal control unit 102 controls the electric switch 1023 to switch from an off mode, in which the accumulator 1016 is not in use, to an on mode, in which the accumulator 1016 supplies energy for the operation of the apparatus 100. The accumulator may be combined with or replaced by a capacitor.

FIG. 676 shows an embodiment identical to that of FIG. 675, except that a battery 1022 also is implanted in the patient and the implanted components are interconnected differently. In response to a control signal from the wireless remote control of the external energy-transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the apparatus 100.

Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the apparatus 100.

It should be understood that the electric switch 1023 and all other switches in this application should be interpreted in its broadest embodiment. This means a transistor, MCU, MCPU, ASIC, FPGA or a DA converter or any other electronic component or circuit that may switch the power on and off.

Preferably the switch is controlled from outside the body, or alternatively by an implanted internal control unit.

FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the apparatus 100 (mechanically operated). Even simpler is to switch the direction of the motor electronically. The gear box interpreted in its broadest embodiment may stand for a servo arrangement saving force for the operation device in favor of longer stroke to act.

FIG. 678 shows an embodiment identical to that of FIG. N40 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the apparatus 100.

FIG. 679 schematically shows conceivable combinations of implanted components of the apparatus for achieving various communication options. Basically, there are the apparatus 100, the internal control unit 102, motor or pump unit 1009, and the external energy-transmission device 200 including the external wireless remote control. As already described above the wireless remote control transmits a control signal which is received by the internal control unit 102, which in turn controls the various implanted components of the apparatus.

A feedback device, preferably comprising a sensor or measuring device 1025, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow.

The feedback may be sent to the internal control unit or out to an external control unit preferably via the internal control unit. Feedback may be sent out from the body via the energy transfer system or a separate communication system with receiver and transmitters.

The internal control unit 102, or alternatively the external wireless remote control of the external energy-transmission device 200, may control the apparatus 100 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver. Alternatively, the wireless remote control may comprise a signal receiver or transceiver and the internal control unit 102 may comprise a signal transmitter or transceiver. The above transceivers, transmitters and receivers may be used for sending information or data related to the apparatus 100 from inside the patient's body to the outside thereof.

Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly.

FIG. 680 shows an alternative embodiment wherein the apparatus 100 is regulated from outside the patient's body. The system comprises a battery 1022 connected to the apparatus 100 via a subcutaneous electric switch 1026. Thus, the regulation of the apparatus 100 is performed non-invasively by manually pressing the subcutaneous switch, whereby the operation of the apparatus 100 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 681 shows an alternative embodiment, wherein the system comprises a hydraulic fluid reservoir 1013 hydraulically connected to the apparatus. Non-invasive regulation is performed by manually pressing the hydraulic reservoir connected to the apparatus. Alternatively, the hydraulic fluid reservoir 1013 is adapted to work with an injection port for the injection of hydraulic fluid, preferably for calibration of hydraulic fluid.

The system may include an external data communicator and an implantable internal data communicator communicating with the external data communicator. The internal communicator feeds data related to the apparatus or the patient to the external data communicator and/or the external data communicator feeds data to the internal data communicator.

FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 1002 connected to implanted energy consuming components of the apparatus 100. Such an energy receiver 1002 may include an energy source and/or an energy-transforming device. Briefly described, wireless energy is transmitted from an external energy source 200a located outside the patient and is received by the internal energy receiver 1002 located inside the patient. The internal energy receiver is adapted to directly or indirectly supply received energy to the energy consuming components of the apparatus 100 via a switch 1026. An energy balance is determined between the energy received by the internal energy receiver 1002 and the energy used for the apparatus 100, and the transmission of wireless energy is then controlled based on the determined energy balance. The energy balance thus provides an accurate indication of the correct amount of energy needed, which is sufficient to operate the apparatus 100 properly, but without causing undue temperature rise.

In FIG. 682 the patient's skin is indicated by a vertical line SK. Here, the energy receiver comprises an energy-transforming device 1002 located inside the patient, preferably just beneath the patient's skin SK. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy source 200a provided in an external energy-transmission device 200 located outside the patient's skin SK in the vicinity of the implanted energy-transforming device 1002.

As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used.

The amount of energy received by the implanted energy receiver may be compared with the energy used by the implanted components of the apparatus. The term “energy used” is then understood to include also energy stored by implanted components of the apparatus. A control device includes an external control unit 200b that controls the external energy source 200a based on the determined energy balance to regulate the amount of transferred energy. In order to transfer the correct amount of energy, the energy balance and the required amount of energy is determined by means of a determination device including an implanted internal control unit 102 connected between the switch 1026 and the apparatus 100. The internal control unit 102 may thus be arranged to receive various measurements obtained by suitable sensors or the like, not shown, measuring certain characteristics of the apparatus 100, somehow reflecting the required amount of energy needed for proper operation of the apparatus 100. Moreover, the current condition of the patient may also be detected by means of suitable measuring devices or sensors, in order to provide parameters reflecting the patient's condition. Hence, such characteristics and/or parameters may be related to the current state of the apparatus 100, such as power consumption, operational mode and temperature, as well as the patient's condition reflected by parameters such as; body temperature, blood pressure, heartbeats and breathing. Other kinds of physical parameters of the patient and functional parameters of the device are described elsewhere.

Furthermore, an energy source in the form of an accumulator 1016 may optionally be connected to the implanted energy-transforming device 1002 via the control unit 102 for accumulating received energy for later use by the apparatus 100. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 100, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics.

For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 102 of the determination device is adapted to determine the energy balance and/or the currently required amount of energy, (either energy per time unit or accumulated energy) based on measurements made by the above-mentioned sensors or measuring devices of the apparatus 100, or the patient, or an implanted energy source if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal.

Alternatively, the determination device may include the external control unit 200b. In this alternative, sensor measurements can be transmitted directly to the external control unit 200b wherein the energy balance and/or the currently required amount of energy can be determined by the external control unit 200b, thus integrating the above-described function of the internal control unit 102 in the external control unit 200b. In that case, the internal control unit 102 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements.

Hence, the present solution according to the arrangement of FIG. 682 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus.

The internal signal transmitter 1027 and the external signal receiver 200c may be implemented as separate units using suitable signal transfer means, such as radio, IR (Infrared) or ultrasonic signals. Alternatively, the internal signal transmitter 1027 and the external signal receiver 200c may be integrated in the implanted energy-transforming device 1002 and the external energy source 200a, respectively, so as to convey control signals in a reverse direction relative to the energy transfer, basically using the same transmission technique. The control signals may be modulated with respect to frequency, phase or amplitude.

Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off. In implementing this system in the arrangement of FIG. 682, the switch 1026 is either separate and controlled by the internal control unit 102, or integrated in the internal control unit 102. It should be understood that the switch 1026 should be interpreted in its broadest embodiment. This means a transistor, MCU, MCPU, ASIC FPGA or a DA converter or any other electronic component or circuit that may switch the power on and off.

To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 1027 to the external signal receiver 200c. Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer.

The amount of transferred energy can generally be regulated by adjusting various transmission parameters in the external energy source 200a, such as voltage, current, amplitude, wave frequency and pulse characteristics.

This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized.

This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil.

The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor.

With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 1026 is interconnected between the external energy source 200a and an operation device, such as an electric motor 1007 operating the apparatus 100. An external control unit 200b controls the operation of the external switch 1026 to effect proper operation of the apparatus 100.

FIG. 684 illustrates different embodiments for how received energy can be supplied to and used by the apparatus 100. Similar to the example of FIG. 682, an internal energy receiver 1002 receives wireless energy E from an external energy source 200a which is controlled by a transmission control unit 200b. The internal energy receiver 1002 may comprise a constant voltage circuit, indicated as a dashed box “constant V” in the figure, for supplying energy at constant voltage to the apparatus 100. The internal energy receiver 1002 may further comprise a constant current circuit, indicated as a dashed box “constant C” in the figure, for supplying energy at constant current to the apparatus 100.

The apparatus 100 comprises an energy consuming part 10j, which may be a motor, pump, restriction device, or any other medical appliance that requires energy for its electrical operation. The apparatus 100 may further comprise an energy storage device 10k for storing energy supplied from the internal energy receiver 1002. Thus, the supplied energy may be directly consumed by the energy consuming part 10j, or stored by the energy storage device 10k, or the supplied energy may be partly consumed and partly stored. The apparatus 100 may further comprise an energy stabilizing unit 101 for stabilizing the energy supplied from the internal energy receiver 1002. Thus, the energy may be supplied in a fluctuating manner such that it may be necessary to stabilize the energy before consumed or stored.

The energy supplied from the internal energy receiver 1002 may further be accumulated and/or stabilized by a separate energy stabilizing unit 1028 located outside the apparatus 100, before being consumed and/or stored by the apparatus 100. Alternatively, the energy stabilizing unit 1028 may be integrated in the internal energy receiver 1002. In either case, the energy stabilizing unit 1028 may comprise a constant voltage circuit and/or a constant current circuit.

It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope of the present invention.

FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window.

The schematic FIG. 685 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 668; the transmitting parts of the system are excluded.

The implementation of the general concept of energy balance and the way the information is transmitted to the external energy transmitter can of course be implemented in numerous different ways.

The schematic FIG. 685 and the above described method of evaluating and transmitting the information should only be regarded as examples of how to implement the control system.

### Circuit Details

In FIG. 685 the symbols Y1, Y2, Y3 and so on symbolize test points within the circuit. The components in the diagram and their respective values are values that work in this particular implementation which of course is only one of an infinite number of possible design solutions.

Energy to power the circuit is received by the energy receiving coil L1. Energy to implanted components is transmitted in this particular case at a frequency of 25 kHz. The energy balance output signal is present at test point Y1.

Those skilled in the art will realize that the above various embodiments of the system could be combined in many different ways. For example, the electric switch 1006 of FIG. 668 could be incorporated in any of the embodiments of FIGS. 671-677, the hydraulic valve shifting device 1014 of

FIG. 671 could be incorporated in the embodiment of FIG. 670, and the gear box 1024 could be incorporated in the embodiment of FIG. 669. Please observe that the switch simply could mean any electronic circuit or component.

The embodiments described in connection with FIGS. 682, 684 and 685 identify a method and a system for controlling transmission of wireless energy to implanted energy consuming components of an electrically operable apparatus. Such a method and system will be defined in general terms in the following.

A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance.

The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied.

When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

In one embodiment at least one battery may be a part of or replace the energy-transforming device 1002 to supply energy to the apparatus 100 over a power supply line. In one embodiment the battery is not rechargeable. In an alternative embodiment the battery is rechargeable. The battery supply may of course be placed both remote to and incorporated in the device.

Further, the system may comprise any of the following:
A primary coil in the external energy source adapted to transmit the wireless energy inductively to a secondary coil in the internal energy receiver.
A determination device adapted to detect a change in the energy balance, wherein the control device controls the transmission of wireless energy based on the detected energy balance change
A determination device adapted to detect a difference between energy received by the internal energy receiver and energy used for the implantable energy consuming components of the apparatus,
wherein the control device controls the transmission of wireless energy based on the detected energy difference.
A control device controlling the external energy-transmission device to decrease the amount of transmitted wireless energy if the detected energy balance change implies that the energy balance is increasing, or vice versa, wherein the decrease/increase of energy transmission corresponds to a detected change rate.
A control device controlling the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
An apparatus wherein the energy used for the apparatus is consumed to operate the apparatus, and/or stored in at least one energy storage device of the apparatus.
An apparatus where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
An apparatus wherein, wherein a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
An apparatus wherein the derivative is determined over time of a measured electrical parameter related to the amount of consumed and/or stored energy, the determination device determines the derivative for a monitored voltage and/or current related to the energy balance.
An energy-transmission device comprising a coil placed externally to the human body, and an electric circuit provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
An electric circuit adapted to deliver the electrical pulses to remain unchanged except varying the first and/or second time intervals.
An electric circuit having a time constant and being adapted to vary the first and second time intervals only in the range of the first time constant, so that when the lengths of the first and/or second time intervals are varied, the transmitted power over the coil is varied.
An electric circuit adapted to deliver the electrical pulses to be varied by only varying the lengths of first time intervals between successive leading and trailing edges of the electrical pulses.
An electric circuit adapted to supply a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
An electric circuit adapted to provide the electrical pulses as pulses having a substantially constant height and/or amplitude and/or intensity and/or voltage and/or current and/or frequency.
An electric circuit having a time constant, and being adapted to vary the first and second time intervals only in the range of the first time constant, so that when the lengths of the first and/or second time intervals are varied, the transmitted power across the first coil are varied.
An electric circuit adapted to provide the electrical pulses varying the lengths of the first and/or the second time intervals only within a range that includes the first time constant or that is located relatively close to the first time constant, compared to the magnitude of the first time constant.

FIGS. 686-689 show in more detail block diagrams of four different ways of hydraulically or pneumatically powering an implanted apparatus according to the invention.

FIG. 686 shows a system as described above with. The system comprises an implanted apparatus 100 and further a separate regulation reservoir 1013, a one way pump 1009 and an alternate valve 1014.

FIG. 687 shows the apparatus 100 and a fluid reservoir 1013. By moving the wall of the regulation reservoir or changing the size of the same in any other different way, the adjustment of the apparatus may be performed without any valve, just free passage of fluid any time by moving the reservoir wall.

FIG. 688 shows the apparatus 100, a two way pump 1009 and the regulation reservoir 1013.

FIG. 689 shows a block diagram of a reversed servo system with a first closed system controlling a second closed system. The servo system comprises a regulation reservoir 1013 and a servo reservoir 1050. The servo reservoir 1050 mechanically controls an implanted apparatus 100 via a mechanical interconnection 1054. The apparatus has an expandable/contactable cavity. This cavity is preferably expanded or contracted by supplying hydraulic fluid from the larger adjustable reservoir 1052 in fluid connection with the apparatus 100. Alternatively, the cavity contains compressible gas, which can be compressed and expanded under the control of the servo reservoir 1050.

The servo reservoir 1050 can also be part of the apparatus itself.

In one embodiment, the regulation reservoir is placed subcutaneous under the patient's skin and is operated by pushing the outer surface thereof by means of a finger. This system is illustrated in FIGS. 690a-690c. In FIG. 690a, a flexible subcutaneous regulation reservoir 1013 is shown connected to a bulge shaped servo reservoir 1050 by means of a conduit 1011. This bellow shaped servo reservoir 1050 is comprised in a flexible apparatus 100. In the state shown in FIG. 690a, the servo reservoir 1050 contains a minimum of fluid and most fluid is found in the regulation reservoir 1013. Due to the mechanical interconnection between the servo reservoir 1050 and the apparatus 100, the outer shape of the apparatus 100 is contracted, i.e., it occupies less than its maximum volume. This maximum volume is shown with dashed lines in the figure.

FIG. 690b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 100 so that it occupies its maximum volume, thereby stretching the stomach wall (not shown), which it contacts.

The regulation reservoir 1013 is preferably provided with means 1013a for keeping its shape after compression. This means, which is schematically shown in the figure, will thus keep the apparatus 100 in a stretched position also when the user releases the regulation reservoir. In this way, the regulation reservoir essentially operates as an on/off switch for the system.

An alternative embodiment of hydraulic or pneumatic operation will now be described with reference to FIGS. 691 and 692a-692c. The block diagram shown in FIG. 691 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 100 having an expandable/contactable cavity is in turn controlled by the larger adjustable reservoir 1052 by supply of hydraulic fluid from the larger adjustable reservoir 1052 in fluid connection with the apparatus 100.

An example of this embodiment will now be described with reference to FIG. 692a-692c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 692a, the servo reservoir 1050 contains a minimum of fluid and most fluid is found in the regulation reservoir 1013.

The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 100. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 100. In other words, in this reversed servo, a small volume in the regulation reservoir is compressed with a higher force and this creates a movement of a larger total area with less force per area unit.

Like in the previous embodiment described above with reference to FIGS. 690a-690c, the regulation reservoir 1013 is preferably provided with means 1013a for keeping its shape after compression. This means, which is schematically shown in the figure, will thus keep the apparatus 100 in a stretched position also when the user releases the regulation reservoir. In this way, the regulation reservoir essentially operates as an on/off switch for the system.

One single volume filling device has been described as invaginated in the stomach wall. Alternatively, two or more volume filling devices 100 may be invaginated to obtain the desired reduction of the food cavity. One such example is illustrated in FIG. 500, wherein three ball-shaped volume filling devices 100 are invaginated in the wall of the patient's stomach 12.

It has been described how the volume filling device 100 is invaginated in the stomach wall by means of a gastroscopic instrument. It will be appreciated that abdominal operation methods can be used as well. Such methods will now be described in detail with reference to FIGS. 501a and 501b.

In a first alternative embodiment, the volume filling device is implanted using a laparoscopic method instead of the intraluminar method described above. According to this embodiment, a needle or a tube-like instrument is inserted into the abdomen of the patient's body, and said needle or tube-like instrument is then used to fill the patient's abdomen with gas. Subsequently, at least two laparoscopic trocars are inserted into the patient's body; and a camera is inserted through one of said at least two laparoscopic trocars. Then, at least one dissecting tool through one of said at least two laparoscopic trocars, and an area of the stomach is dissected. The volume filling device is then introduced into the abdominal cavity, and placed on the outside of the stomach wall. A pouch in the stomach wall for the device is created, and the device invaginated in said pouch by providing sutures or staples to the stomach wall, thereby positioning the volume filling device so that the volume of the food cavity is reduced in size by a volume substantially exceeding the volume of the volume filling device.

The above first alternative preferably further comprises affixing the device to the stomach wall by providing sutures or staples.

The above embodiment preferably further comprises providing an apparatus for regulating the obesity treatment device from the outside of the patient's body; and operating said apparatus to regulate the obesity treatment device. Further, regulation of the obesity treatment device includes changing the volume of a filling body of the volume filling device when implanted.

The above embodiment preferably further comprises providing an injection type syringe comprising a fluid for injection into an implanted filling body; and injecting volume of fluid into said filling body.

According to an embodiment, the device is enclosed in the pouch or partially enclosed in that the pouch is left at least partly open. Further, the pouch can be designed to exhibit only one opening. Alternatively the pouch is designed to exhibit two openings and to extend non-circumferentially around the stomach. Preferably the pouch has a volume of more than 15 milliliters.

In a second alternative, also using a laparoscopic method instead of the intraluminar method, the initial steps are the same as described in the first alternative, but following dissection of the stomach, a hole is created in the stomach wall and a volume filling device introduced into the abdominal cavity and through said hole into the stomach. The device is placed on the inside of the stomach wall, and a pouch is created on the outside of the stomach cavity for the device placed on the inside of the stomach wall, and the device is invaginated in the pouch by providing sutures or staples to the stomach wall, thereby positioning the volume filling device so that the volume of the food cavity is reduced in size by a volume substantially exceeding the volume of the volume filling device.

The above embodiment preferably further comprises affixing the device to the stomach wall by providing sutures or staples. According to one embodiment, the stomach wall is affixed to the lower part of the patient's esophagus by providing sutures or staples.

The above second alternative preferably further comprises providing an apparatus for regulating the obesity treatment device from the outside of the patient's body; and operating said apparatus to regulate the obesity treatment device. Further, regulation of the obesity treatment device includes changing the volume of a filling body of the volume filling device when implanted.

The above embodiment preferably further comprises providing an injection type syringe comprising a fluid for injection into an implanted filling body; and injecting volume of fluid into said filling body.

According to an embodiment, the device is enclosed in the pouch or partially enclosed in that the pouch is left at least partly open. Further, the pouch can be designed to exhibit only one opening. Alternatively the pouch is designed to exhibit two openings and to extend non-circumferentially around the stomach. Preferably the pouch has a volume of more than 15 milliliters.

A third alternative involves a surgical incision instead of the either the intraluminar or the laparoscopic method. Here, an opening in the patient's abdominal wall is made by surgical incision, and an area of the patient's stomach is dissected. The volume filling device is introduced through said abdominal incision, and attached to the stomach wall, thereby positioning the volume filling device so that the volume of the food cavity is reduced in size by a volume substantially exceeding the volume of the volume filling device.

In an alternative embodiment of the above, third alternative, the initial steps are the same including the dissection of an area of the stomach. Following this, a pouch in the stomach wall is created for the device, and the device invaginated in the pouch by providing sutures or staples to the stomach wall, thereby positioning the volume filling device so that the volume of the food cavity is reduced in size by a volume substantially exceeding the volume of the volume filling device.

In yet another alternative embodiment of the above, third alternative, the initial steps are the same including the dissection of an area of the stomach. Following this, a hole in the stomach wall is created and the volume filling device introduced through the hole and into the stomach. The device is then placed on the inside of the stomach wall, and a pouch on the stomach wall created for the device. The device is then invaginated in the pouch by providing sutures or staples to the stomach wall, thereby positioning the volume filling device so that the volume of the food cavity is reduced in size by a volume substantially exceeding the volume of the volume filling device.

The above embodiments of the third alternative further comprise affixing the device to the stomach wall by providing sutures or staples.

The above embodiment preferably further comprises providing an apparatus for regulating the obesity treatment device from the outside of the patient's body; and operating said apparatus to regulate the obesity treatment device. Further, regulation of the obesity treatment device includes changing the volume of a filling body of the volume filling device when implanted.

The above embodiment preferably further comprises providing an injection type syringe comprising a fluid for injection into an implanted filling body; and injecting volume of fluid into said filling body.

According to an embodiment, the device is enclosed in the pouch or partially enclosed in that the pouch is left at least partly open. Further, the pouch can be designed to exhibit only one opening. Alternatively the pouch is designed to exhibit two openings and to extend non-circumferentially around the stomach.

Preferably the pouch has a volume of more than 15 milliliters.

A fourth alternative embodiment is a method comprising the steps of inserting a needle or a tube-like instrument into the abdomen of the patient's body; using said needle or tube-like instrument to fill the patient's abdomen with gas; placing at least two laparoscopic trocars in the patient's body; inserting a camera through one of said at least two laparoscopic trocars into the patient's abdomen; inserting at least one dissecting tool through one of said at least two laparoscopic trocars; dissecting an area of the stomach; creating a pouch from the stomach wall for the device; closing the pouch by providing sutures and staples; introducing a injecting member comprising an injectable filling material; and injecting the filling material into the pouch, thereby creating a filling body that fills a volume in the patient's stomach, reducing the food cavity in size by a volume substantially exceeding the volume of the volume filling device.

Instead of the above disclosed laparoscopic method, a surgical incision or opening is cut in the skin to enter the patient's abdomen; an area of the stomach dissected; a pouch created from the stomach wall for the device; and said pouch closed by providing sutures and staples. An injecting member comprising an injectable filling material is then introduced; and the filling material injected into the pouch, thereby creating a filling body that reduces the food cavity in size by a volume substantially exceeding the volume of the volume filling device.

According to an alternative embodiment of the above, the pouch is created on the outside of the stomach wall, with the filling body placed against the inside of the stomach wall.

The method according to either of the two previous embodiments comprises creating a hole in the stomach wall wherein the pouch is created on the inside of the stomach wall, with the filling body placed against the outside of the stomach wall.

The method according to either of the two previous embodiments may further comprise affixing the stomach wall to the lower part of the patient's esophagus by providing sutures or staples or affixing the stomach wall to the patient's diaphragm muscle or associated muscles.

Preferably the pouch has a volume of more than 15 milliliters.

In a method according to either of the two previous embodiments the filling material is preferably capable of undergoing a curing process from a fluid state to a semi-solid or solid state. Preferably said curing process is triggered by an increase in temperature from ambient temperature to body temperature.

The invention also makes available a method of treating obesity in a patient by implanting a volume filling device that, when implanted in a patient, reduces the food cavity in size by a volume substantially exceeding the volume of the volume filling device, the method comprising the steps of inserting a needle or a tube-like instrument into the abdomen of the patient's body; using said needle or tube-like instrument to fill the patient's abdomen with gas; placing at least two laparoscopic trocars in the patient's body; inserting a camera through one of said at least two laparoscopic trocars into the patient's abdomen; inserting at least one dissecting tool through one of said at least two laparoscopic trocars; dissecting an area of the stomach; creating a hole in the stomach wall; introducing a device into the abdominal cavity; introducing the device through the hole and into the stomach; placing the device on the outside of the stomach wall; fixating the device placed on the outside of the stomach wall, and thereby creating a filling body that reduces the food cavity in size by a volume substantially exceeding the volume of the volume filling device. In the above method, the device is preferably affixed to the stomach wall by providing sutures or staples.

The invention also comprises a laparoscopic instrument for providing a volume filling device to be invaginated in the stomach wall of a human patient to treat obesity, suitable for use with any of the laparoscopic methods described above, the instrument comprising: an elongated member having a proximal end and a distal end, the elongated member having a diameter less than that of a laparoscopic trocar to be introduced into the patients abdomen during a laparoscopic operation; a stomach pushing device for pushing the stomach wall to create a tube-like shaped portion of the stomach wall protruding into the normal stomach cavity, said pushing device comprising the volume filling device to be invaginated by the stomach wall in the tube-like shaped portion thereof; wherein the pushing device comprises a vacuum device to suck the stomach fundus to assist the instrument in forming the tube-like shaped portion of the stomach wall together with the pushing device, and wherein the vacuum device comprises a vacuum passageway leading from the proximal to the distal end of the instrument and at the end portion of the instrument, which includes the pushing device, said vacuum passageway is divided up in multiple small openings adapted to suck the stomach wall portion to become adherent to the pushing device to further form the tube-like stomach wall portion; and wherein the instrument comprises an insertion device adapted to introduce the volume filling device into the tube-like shaped stomach portion.

This instrument preferably comprises at least one clamping device for holding the opening of the tube-like portion substantially closed by clamping together stomach to stomach in said opening, wherein the instrument is adapted to place the at least one clamping device at the opening in such a way that it allows later suturing of the opening.

Further, the instrument preferably comprises an inflation device for inflating the volume filling device before or after the suturing. Further still, the instrument preferably comprises a suturing device adapted to suture the opening of the tube-like portion with stomach-to-stomach sutures for creating at least partly a closed space enclosing the volume filling device, wherein the instrument is adapted to be withdrawn leaving the volume filling device at least partly invaginated in the stomach wall.

Said suturing device preferably comprises a first and second suture positioning member provided on the elongated member situated in the stomach at the distal end thereof, and wherein the instrument further comprises an operation device adapted to adjust the first and second suturing member in a position in which the first and second suture positioning members are in front of each other with the stomach wall on both sides of the open end of the cup like portion, and adapted to suture the open end of the cup like portion of the wall with a row of stomach to stomach sutures.

Preferably said suturing device comprises an operable re-loadable multi-suturing device, which is reloadable with sutures from outside of the patient's body and which is adapted to suture the open end of the cup like portion of the wall with said row of stomach-to-stomach sutures, wherein the row of sutures comprises two or more sutures or staples to be sutured simultaneously.

More preferably, said suturing device comprises multiple sutures for suturing two or more sutures simultaneously.

### O, P: Egg and/or Embryo Control of a Female Patient to Avoid and/or Promote Pregnancy

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for pregnancy control of a female patient. The control unit/controller, control systems, communication systems, housing/fixation units and methods could also be used for controlling, communicating with and/or operating a device for embryo control of a female patient. Examples of such devices for pregnancy/embryo control of a female patient will now be described.

Referring to the drawing figures, like reference numerals designate identical or corresponding elements throughout the several figures.

FIGS. 502A and 502B illustrates a first embodiment of a device 100 for treating a female patient to prevent/promote pregnancy applied on the oviducts 31a, 31b of a female patient. Clamping elements 5, 6 of a restriction or constriction device 2 constrict the oviducts 31a, 31b. (For the sake of clarity, the housing is not shown and the clamping elements 5, 6 are exaggerated.) In this embodiment, a control device 102 includes a subcutaneously implanted push button that is manually switched by the patient between “on” and “off”. This control device will be described in more detail below with reference to FIGS. 504A AND 504B. Such a manually operable push button may also be provided in combination with a remote control as an emergency button to allow the patient to stop the operation of the device in case of emergency or malfunction. Such a remote control will be described below with reference to FIGS. 503A and 503B. This device could also be fully manually controlled by manual manipulation of for example a hydraulic reservoir controlling a (restriction) hydraulic device such as described in FIG. 504A-504D or 517A and 517B or 512A and 512B or any of 513A-513D. Please observe that only a reservoir moving fluid manually as in FIGS. 504C and 504D may be used to adjust the (restriction) device. This reservoir may be controlled in such a way that the change of the reservoir volume will be stable after the manual manipulation of the reservoir wall has taken place. In this particular case is showed a looking device but many different ways may be used, small amount of fluid in the reservoir may also cause a larger movement of the (restriction) device as described in some of the embodiments above.

FIGS. 503A and 503B illustrates an alternative embodiment applied on the oviducts 31a, 31b of a female patient. The clamping elements 5, 6 of the constriction device 2 constrict the oviducts 31a, 31b.

(For the sake of clarity, the housing is not shown and the clamping elements 5, 6 are exaggerated.) In this embodiment, a control device includes an external control unit in the form of a hand-held wireless remote control 200, and an implanted internal control unit 102, which may include a microprocessor, for controlling the constriction and stimulation devices. The remote control 200 is operable by the patient to control the internal control unit 102 to switch on and off the (restriction) device. The internal control unit 102 controls an implanted operation device 34 to move the clamping elements 5, 6. An implanted source of energy 35, such as a rechargeable battery, powers the operation device 34. The internal control unit 102, which may be implanted subcutaneously or in the abdomen, may also work as an energy receiver, i.e., for transforming wireless energy into electric energy and charging the implanted source of energy 35 (rechargeable battery) with the electric energy.

An implanted sensor 36 (not shown) senses a physical parameter of the patient, such as the temperature, wherein the internal control unit 102 controls the constriction device 2 and/or the stimulation device 3 (not shown) in response to signals from the sensor 36. In this embodiment the sensor 36 is a hormone level sensor, wherein the internal control unit 102 controls the constriction device and/or stimulation device to change the constriction of the patient's oviduct 31 in response to the sensor 36 sensing a predetermined value of measured value. For example, the control unit 102 may control the constriction device and/or stimulation device to increase the constriction of the patient's oviduct 31 in response to the sensor sensing an increased or decreased hormone level. Alternatively or in combination, the remote control 32 controls the constriction device and/or a stimulation device in response to signals from the sensor 36, in the same manner as the internal control unit 102.

The remote control 200 may be equipped with means for producing an indication, such as a sound signal or displayed information, in response to signals from the sensor 36. When the patient's attention is taken by such an indication indicating a release of the oviduct based on said sensor input. The patient may use the remote control to control the constriction device or stimulation device to pump eggs through the oviducts of the patient. FIGS. 504A and 504B show hydraulic operation means suited for operating the constriction device of the embodiments described above with reference to FIGS. 502A and 502B. Specifically, FIGS. 504A and 504B show the device of FIGS. 502A and 502B provided with such means for hydraulic operation of the constriction device 2. Thus, the housing 1 forms two hydraulic chambers 22a and 22b, in which the two clamping elements 5, 6 are slidable back and forth relative to the tubular tissue wall portion 8 of a patient's oviduct. The hydraulic operation means include an expandable reservoir 23, such as an elastic balloon, containing hydraulic fluid, conduits 24a and 24b between the reservoir 23 and the hydraulic chambers 22a, 22b, and a two-way pump 25 for pumping the hydraulic fluid in the conduits 24a, 24b. The reservoir 23 is subcutaneously placed between the skin, shown with a solid line in the figure, and the normally fascial/muscular layer, shown with a dashed line. The control device 4 controls the pump 25 to pump hydraulic fluid from the reservoir 23 to the chambers 22a, 22b to move the clamping elements 5, 6 against the wall portion 8, whereby the tubular wall portion 8 is constricted, see FIG. 504B, and to pump hydraulic fluid from the chambers 22a, 22b to the reservoir 23 to move the clamping elements 5, 6 away from the wall portion 8, whereby the tubular wall 8 is released, see FIG. 504A.

Alternatively, the embodiment of FIGS. 504A and 504B may be manually operated by applying suitable manually operable hydraulic means for distributing the hydraulic fluid between the expandable reservoir 23 and the hydraulic chambers 22a, 22b. In this case the pump 25 is omitted. Also in this case is the control device 4 only manual manipulation of the reservoir.

FIGS. 505A and 505B schematically show a mechanically operable embodiment of the invention, comprising an open ended tubular housing 26 applied on the tubular tissue wall portion 8 of a patient's organ, a constriction device 27 arranged in the housing 26 and a control device 4 for controlling the constriction device 27. A stimulation device (not shown) as described above is also provided in the housing 26. The constriction device 27 includes a clamping element 28, which is radially movable in the tubular housing 26 towards and away from the tubular wall portion 8 between a retracted position, see FIG. 505A, and a clamping position, see FIG. 505B, in which the clamping element 28 gently constricts the tubular wall portion 8. Mechanical operation means for mechanically operating the clamping element 28 includes an electric motor 29 attached to the housing 26 and a telescopic device 30, which is driven by the motor 29 and operatively connected to the clamping element 28. The control device 4 controls the electric motor 29 to expand the telescopic device 30 to move the clamping element 28 against the wall portion 8, whereby the tubular wall portion 8 is constricted, see FIG. 505B, and controls the motor 29 to retract the telescopic device 30 to move the clamping element 28 away from the wall portion 8, whereby the wall portion 8 is released, see FIG. 505A.

Alternatively, the motor 29 may be omitted and the telescopic device 30 be modified for manual operation, as shown in FIG. 505a. Thus, a spring 30a may be provided acting to keep the telescopic device 30 expanded to force the clamping element 28 against the wall portion 8. The mechanical operation means may include a subcutaneously implanted lever mechanism 29a that is operatively connected to the telescopic device 30. The patient may push the lever mechanism 29a through the patient's skin 29b to pull the telescopic device 30 against the action of the spring 30a to the retracted position of the telescopic device 30, as indicated in phantom lines. When the patient releases the lever mechanism 29a, the spring 30a expands the telescopic device 30, whereby clamping element 28 is forced against the wall portion 8.

FIGS. 506A-506C illustrate in principle the function of the system of FIG. 502 when the device is applied on a portion 8 of a tubular tissue wall of a patient's oviduct. Thus, FIG. 506A shows the device in a non-clamping state, in which the clamping elements 5, 6 are in their retracted positions and the wall portion 8 extends through the open ends of the housing 1 without being constricted by the clamping elements 5, 6. FIG. 506B shows the device in a clamping state, in which the clamping elements 5, 6 have been moved from their retracted positions to their clamping positions, in which the clamping elements 5, 6 gently constrict the wall portion 8 to a constricted state, in which the blood circulation in the constricted wall portion 8 is substantially unrestricted and the flow in the oviduct of the wall portion 8 is restricted. FIG. 506C shows the device in an optional stimulation state, in which the clamping elements 5, 6 constrict the wall portion 8 and the electrical elements 7 of the stimulation device 3 electrically stimulate different areas of the wall portion 8, so that the wall portion 8 contracts (thickens) and closes the oviduct.

When the system is in its optional stimulation state, it is important to stimulate the different areas of the wall portion 8 in a manner so that they essentially maintain their natural physical properties over time to prevent the areas from being injured. Consequently, the control device 4 controls the stimulation device 3 to intermittently stimulate each area of the wall portion 8 during successive time periods, wherein each time period is short enough to maintain over time satisfactory blood circulation in the area. Furthermore, the control device 4 controls the stimulation of the areas of the wall portion 8, so that each area that currently is not stimulated restores substantially normal blood circulation before it is stimulated again. To maintain over time the effect of stimulation, i.e., to keep the oviduct closed by maintaining the wall portion 8 contracted, the control device 4 controls the stimulation device 3 to stimulate one or more of the areas at a time and to shift the stimulation from one area to another over time. The control device 4 may control the stimulation device 3 to cyclically propagate the stimulation of the areas along the tubular wall portion 8, for example, in accordance with a determined stimulation pattern. To achieve the desired reaction of the tissue wall during the stimulation thereof, the control device may control the stimulation device to, preferably cyclically, vary the intensity of the stimulation of the wall portion 8.

It will be appreciated that the fully restricted state shown in FIG. 506C can be obtained by purely mechanical means not involving electrical stimulation.

FIGS. 507A-506C show another embodiment which includes a tubular housing 9 and three elongate clamping elements 10a, 10b, 10c, which are radially movable in the tubular housing 9 towards and away from a central axis thereof between retracted positions, see FIG. 507A, and clamping positions, see FIG. 507B. The three clamping elements 10a-10c are symmetrically disposed around the central axis of the housing 9. The stimulation device of this embodiment includes electrical elements 11a, 11b, 11c that form a series of groups of elements extending longitudinally along the elongate clamping elements 10a-10c, wherein the electrical elements 11a-11c of each group of electrical elements form a path of three electrical elements 11a, 11b and 11c extending circumferentially around the central axis of the housing 9. The three electrical elements 11a-11c of each group are positioned on the three clamping elements 10a-10c, respectively. Thus, the path of three electrical elements 11a-11c extends around the patient's organ. Of course, the number of electrical elements 11a-11c of each path of electrical elements can be greater than three, and several parallel rows electrical elements 11a-11c can form each path of electrical elements.

FIGS. 508A, 508B and 508C schematically illustrate different states of operation of a generally designed device according to the present invention, when the device is applied on a wall portion of an oviduct designated BO. The system includes a constriction device and a stimulation device, which are designated CSD, and a control device designated CD for controlling the constriction and stimulation devices CSD. FIG. 508A shows the device in an inactivation state, in which the constriction device does not constrict the organ BO and the stimulation device does not stimulate the organ BO. FIG. 508B shows the device in a constriction state, in which the control device CD controls the constriction device to gently constrict the wall portion of the organ BO to a constricted state, in which the blood circulation in the constricted wall portion is substantially unrestricted and the flow in the oviduct of the wall portion is restricted. FIG. 508C shows the device in a stimulation state, in which the control device CD controls the stimulation device to stimulate different areas of the constricted wall portion, so that almost the entire wall portion of the organ BO contracts (thickens) and closes the oviduct.

FIGS. 508D and 508E show how the stimulation of the constricted wall portion can be cyclically varied between a first stimulation mode, in which the left area of the wall portion (see FIG. 508D) is stimulated, while the right area of the wall portion is not stimulated, and a second stimulation mode, in which the right area of the wall portion (see FIG. 508E) is stimulated, while the left area of the wall portion is not stimulated, in order to maintain over time satisfactory blood circulation in the constricted wall portion.

It should be noted that the stimulation modes shown in FIGS. 508D and 508E only constitute a principal example of how the constricted wall portion of the organ BO may be stimulated. Thus, more than two different areas of the constricted wall portion may be simultaneously stimulated in cycles or successively stimulated. Also, groups of different areas of the constricted wall portion may be successively stimulated.

FIGS. 509A, 509B and 509C illustrate different states of operation of a modification of the general embodiment shown in FIGS. 508A-508E, wherein the constriction and stimulation devices CSD include several separate constriction/stimulation elements, here three elements CSDE1, CSDE2 and CSDE3. FIG. 509A shows how the element CSDE1 in a first state of operation is activated to both constrict and stimulate the organ BO, so that the oviduct of the organ BO is closed, whereas the other two elements CSDE2 and CSDE3 are inactivated. FIG. 509B shows how the element CSDE2 in a second following state of operation is activated, so that the oviduct of the organ BO is closed, whereas the other two elements CSDE1 and CSDE3 are inactivated. FIG. 509C shows how the element CSDE3 in a following third state of operation is activated, so that the oviduct of the organ BO is closed, whereas the other two elements CSDE1 and CSDE2 are inactivated. By shifting between the first, second and third states of operation, either randomly or in accordance with a predetermined sequence, different portions of the organ can by temporarily constricted and stimulated while maintaining the oviduct of the organ closed, whereby the risk of injuring the organ is minimized. It is also possible to activate the elements CSDE1-CSDE3 successively along the oviduct of the organ to move fluids and/or other bodily matter in the oviduct.

FIGS. 509D, 509E and 509F illustrate an alternative mode of operation of the modification of the general embodiment. Thus, FIG. 509D shows how the element CSDE7 in a first state of operation is activated to both constrict and stimulate the organ BO, so that the oviduct of the organ BO is closed, whereas the other two elements CSDE2 and CSDE3 are activated to constrict but not stimulate the organ BO, so that the oviduct of the organ BO is not completely closed where the elements CSDE2 and CSDE3 engage the organ BO. FIG. 509E shows how the element CSDE2 in a second following state of operation is activated to both constrict and stimulate the organ BO, so that the oviduct of the organ BO is closed, whereas the other two elements CSDE7 and CSDE3 are activated to constrict but not stimulate the organ BO, so that the oviduct of the organ BO is not completely closed where the elements CSDE1 and CSDE3 engage the organ BO. FIG. 509F shows how the element CSDE3 in a following third state of operation is activated to both constrict and stimulate the organ BO, so that the oviduct of the organ BO is closed, whereas the other two elements CSDE1 and CSDE2 are activated to constrict but not stimulate the organ BO, so that the oviduct of the organ BO is not completely closed where the elements CSDE1 and CSDE2 engage the organ BO. By shifting between the first, second and third states of operation, either randomly or in accordance with a predetermined sequence, different portions of the organ can by temporarily stimulated while maintaining the oviduct of the organ closed, whereby the risk of injuring the organ is reduced. It is also possible to activate the stimulation of the elements CSDE1-CSDE3 successively along the oviduct of the organ BO to move fluids and/or other bodily matter in the oviduct.

This embodiment, with a combination of a mechanical or hydraulic partly restricting system and a stimulation system varying the stimulation position, is preferably used when one wants to close the oviduct for a longer period of time and/or in a safe way. The system is energy efficient because preferably only stimulation needs to be changed in position and the oviduct is allowed to recover until the stimulation comes back to the same position again. If the restriction areas are placed closer to each other and maybe the mechanical or hydraulic restriction is continuous, a peristaltic like wave could be created in any direction in the oviduct. If the restriction is moved in consecutive order starting with the restriction closest to the ovary, no egg may be squeezed in the restriction area. If restriction is moved in the other direction towards the uterus, a movement device could be used to move any egg away from any new upcoming restriction area. Such a movement device could be the hydraulic or mechanical partly restricting device causing movements in the oviduct wall before the stimulation device close the area.

FIG. 510B is a pulse/time diagram showing a modification of the electric stimulation shown in FIG. 510A. Thus, the pulse combination of FIG. 510A is mixed with a pulse train combination having a first relatively long pulse train PTL of high frequency/low amplitude pulses, appearing simultaneously with the positive pulse PL of the pulse combination of FIG. 510A, and a second relatively short pulse train PTS of high frequency/low amplitude appearing simultaneously with the negative pulse PS of the pulse combination shown in FIG. 510A. As a result, the high frequency/low amplitudes pulse trains PTL and PTS are superimposed on the positive and negative pulses PL and PS of FIG. 510A, as illustrated in FIG. 510B. The pulse configuration of FIG. 510B, and variations thereof, is beneficial to use in connection with the stimulation of particular human organs, in order to achieve the desired stimulation effect.

Preferably, the electric pulses form pulse trains, as illustrated in the Pulse/time diagrams P/t of FIGS. 510C and 510D. The Pulse/time diagram P/t of FIG. 511a represents an individual area of the wall portion of the patient's tubular organ which is stimulated with a pulse train 18A. The pulse train 18A includes three initial negative pulses, each of which is of short duration and high amplitude (voltage), and one positive pulse of long duration and low amplitude following the negative pulses. After a delay to enable the area of the organ to restore substantially normal blood circulation, the pulse train 18A is repeated.

The Pulse/time diagram P/t of FIG. 510D represents another individual area of the wall portion, which is stimulated with a pulse train 18B having the same configuration as the pulse train 18A. The pulse trains 18A and 18B are shifted relative to each other, so that they partially overlap one another to ensure that the constricted wall portion always is stimulated to contract as desired.

FIGS. 511a and 511B show another embodiment that controls blood flow in a blood vessel 19, comprising a constriction device with two clamping elements 20a and 20b, a stimulation device in the form of two thermal stimulation elements 21a and 21b integrated in the clamping elements 20a, 20b, respectively, and a control device 4 for controlling the clamping elements 20a, 20b and stimulation elements 21a, 21b. The clamping elements 20a and 20b are movable towards and away from each other in the same manner as described above in connection with the embodiment according to FIGS. 506A-506C. The thermal stimulation elements 21a and 21b, which may include Pertier elements, are positioned on the clamping elements 20a, 20b, so that the thermal elements 21a are facing the thermal elements 21b. FIG. 513A shows how the clamping elements 20a, 20b constrict the blood vessel 19, so that the blood flow is restricted. FIG. 513B shows how the control device 4 controls the thermal stimulation elements 21a, 21b to cool the wall of the blood vessel 19, so that the wall contracts and closes the blood vessel 19. To release the blood vessel 19, the control device 4 controls the thermal stimulation elements 21a, 21b to heat the wall of the blood vessel 19, so that the wall expands.

FIGS. 512A and 512B show a hydraulically operable elongated constriction device in the form of a band 72 having an expandable/contractible cavity 73, which is in fluid communication with an adjustable reservoir 74 containing hydraulic fluid. FIG. 512A illustrates when the band is in a non-constriction state, whereas FIG. 512B illustrates when the band is in a constriction state, in which the cavity 73 is expanded by hydraulic fluid supplied by the reservoir 74.

FIGS. 513A, 513B, 513C and 513D are block diagrams of four differently operated hydraulic constriction devices. FIG. 513A shows the band 72 of FIG. 512A, the cavity 73 of which is in fluid communication with a reservoir 75. FIG. 513B shows the embodiment of FIG. 512A, in which the cavity 73 of the band 72 is in fluid communication with the reservoir 74 via an operation device in the form of a two-way pump 76. FIG. 513C shows an operation device in the form of a reverse servo device with a first closed device controlling a second system. The reverse servo device comprises an adjustable fluid supply reservoir 77 and an adjustable servo reservoir 78. The servo reservoir 78 controls a larger adjustable reservoir 79 which in connection with the band 72 applied around a portion of tubular tissue wall of a patient's organ varies the volume of the cavity 73 of the band 72, which in turn varies the constriction of the wall portion. FIG. 513D shows an embodiment identical to the embodiment of FIG. 513C, except that the larger reservoir 79 is omitted. Instead, the servo reservoir 78 is in fluid communication with the cavity of the band 72.

In all of the above embodiments according to FIGS. 512A through 513D, stimulation devices may be provided to form constriction/stimulation units, in which the stimulation devices include a multiplicity of electrical elements 7 positioned on the constriction devices.

FIG. 514 is a cross-sectional view of a fluid supply device including a bellows reservoir 80 defining a chamber 81, the size of which is variable by an operation device comprising a remote controlled electric motor 82. The reservoir 80 and the motor 82 are placed in a housing 83. Moving a large wall 84 varies the chamber 81. The wall 84 is secured to a nut 85, which is threaded on a rotatable spindle 86. The spindle 86 is rotated by the motor 82. A battery 89 placed in the housing 83 powers the motor 82. A signal receiver 90 for controlling the motor 82 is also placed in the housing 83. Alternatively, the battery 89 and the signal receiver 90 may be mounted in a separate place. The motor 82 may also be powered with energy transferred from transmitted signals.

Where applicable, the fluid supply device of FIG. 514 may be used for supplying hydraulic fluid for the operation of the constriction devices described in this specification. For example, the fluid supply device of FIG. 514 may be substituted for the reservoir 74 in the embodiment according to FIG. 512A.

FIGS. 515A and 515B show a reverse servo including a rectangular housing 91 and an intermediate wall 92, which is movable in the housing 91. A relatively large, substantially cylindrical bellows reservoir 93 is arranged in the housing 91 and is joined to the movable intermediate wall 92. Another cylindrical bellows reservoir 94, which is substantially smaller than reservoir 93, is arranged in the housing 91 at the other side of the intermediate wall 92 and is also joined to the wall 92. The small bellows reservoir 94 has a fluid supply pipe 95 and the large bellows reservoir 93 has a fluid supply pipe 96.

Referring to FIG. 515A, when a small amount of hydraulic fluid is conducted through the supply pipe 95 into the small bellows reservoir 94, the small bellows reservoir 94 expands and pushes the movable intermediate wall 92 towards the large bellows reservoir 93. As a result, the large bellows reservoir 93 is contracted by the intermediate wall 92, whereby a large amount of hydraulic fluid is forced out of the large bellows reservoir 93 through the supply pipe 96, as shown in FIG. 515B.

For example, the reverse servo of FIGS. 515A and 515B may be used in the embodiment of FIG. 513C, wherein the small bellows reservoir 94 corresponds to the small servo reservoir 78 and the large bellows reservoir 93 corresponds to the large reservoir 79. Also, the reverse servo of FIGS. 515A and 515B may be used in the embodiment of FIGS. 512A and 10B, wherein the small bellows reservoir 94 is connected to the adjustable reservoir 74, and the large bellows reservoir 93 is connected to the cavity 73 of the band 72.

FIG. 516 schematically shows a hydraulically operable constriction device 97 of the device of the invention, which is similar to the embodiment shown in FIG. 512A, except that the hydraulic device is designed differently. Thus, the constriction device 97 includes a relatively small inflatable cavity 98, which is in fluid communication with a reservoir 99 containing hydraulic fluid, and a relatively large cavity CL, which is displaceable by small cavity 98. Small cavity 98 is adapted to displace large cavity CL to constrict the patient's tubular wall portion when small cavity 98 is inflated and to displace large cavity CL to release the wall portion when small cavity 98 is deflated. Thus, a relatively small addition of hydraulic fluid from reservoir 99 to small cavity 98 causes a relatively large increase in the constriction of the wall portion.

Large cavity CL is defined by a contraction element in the form of a big balloon 101, which may be connected to an injection port (not shown) for calibration of the volume of large cavity CL. Adding fluid to or withdrawing fluid from the injection port with the aid of a syringe calibrates the volume of balloon 101. Small cavity 98 is defined by a small bellows 102 attached to an annular frame 103 of constriction device 97 and at the opposite end is attached to balloon 101. FIGS. 517A and 517B schematically illustrate the operation of constriction device 97, when annular frame 103 is applied around the tubular wall portion of the patient's organ. Referring to FIG. 517A, when small cavity 98 is deflated bellows 102 pulls balloon 101 inwardly into annular frame 103, so that constriction device 97 constricts the wall portion. Referring to FIG. 517B, when small cavity 98 is inflated bellows 102 pulls balloon 101 out of annular frame 103, so that constriction device 97 releases the wall portion. As mentioned above, the constriction device and stimulation device can co-operate to actively move the egg in the oviduct of a patient's organ. This can be achieved using the constriction/stimulation unit shown in FIG. 503. Thus, in accordance with a first cooperation option, the clamping elements 5, 6 of the constriction device constricts the wall portion 8 without completely closing the oviduct, whereby the flow in the oviduct is restricted, and the control device 4 controls the electrical elements 7 to progressively stimulate the constricted wall portion in the downstream or upstream direction of the oviduct to cause progressive contraction of the wall portion 8 to move the egg in the oviduct. In accordance with a second cooperation option, the constriction device constricts the wall portion so that the flow in the oviduct is restricted, and the control device 4 controls a few electrical elements 7 at one end of the elongate clamping elements 5, 6 to stimulate the constricted wall portion 8 to close the oviduct either at an upstream end or a downstream end of the wall portion 8. With the oviduct closed in this manner, the control device 4 controls the constriction device to increase the constriction of the wall portion, whereby the egg in the oviduct is moved downstream or upstream of the wall portion 8. In another embodiment for performing the second cooperation option, the constriction device constricts the wall portion so that the flow in the oviduct is restricted, and the control device 4 controls the stimulation device to stimulate the constricted wall portion while the constriction device varies the constriction of the different areas of the wall portion, such that the wall portion is progressively constricted in the downstream or upstream direction of the oviduct. FIG. 518 schematically shows a general embodiment of the device of the invention, in which energy is transferred to energy consuming components of the device implanted in the patient. The device of FIG. 518 comprises an implanted constriction/stimulation unit 110, which is operable to gently constrict a portion of a tubular tissue wall of a patient's organ and to stimulate different areas of the constricted portion to cause contraction of the wall portion. The constriction device of the constriction/stimulation unit 110 is capable of performing a reversible function, i.e., to constrict and release the wall portion, so that the constriction/stimulation unit 110 works as an artificial sphincter. A source of energy 111 is adapted to supply energy consuming components of the constriction/stimulation unit 110 with energy via a power supply line 112. A wireless remote control or a subcutaneously implanted switch operable by the patient to switch on or off the supply of energy from the source of energy may be provided. The source of energy may be an implantable permanent or rechargeable battery, or be included in an external energy-transmission device, which may be operable directly by the patient or be controlled by a remote control operable by the patient to transmit wireless energy to the energy consuming components of the constriction/stimulation unit. Alternatively, the source of energy may comprise a combination of an implantable rechargeable battery, an external energy-transmission device and an implantable energy-transforming device for transforming wireless energy transmitted by the external energy-transmission device into electric energy for the charge of the implantable rechargeable battery. FIG. 519 shows a special embodiment of the general embodiment of FIG. 518 having some parts implanted in a patient and other parts located outside the patient's body. Thus, in FIG. 519 all parts placed to the right of the patient's skin 109 are implanted and all parts placed to the left of the skin 109 are located outside the patient's body. An implanted energy-transforming device 111A of the device is adapted to supply energy consuming components of the constriction/stimulation unit 110 with energy via the power supply line 112. An external energy-transmission device 111 of the system includes a wireless remote control transmitting a wireless signal, which is received by a signal receiver incorporated in the implanted energy-transforming device 111A. The implanted energy-transforming device 111A transforms energy from the signal into electric energy, which is supplied via the power supply line 112 to the constriction/stimulation unit 110. The device of FIG. 519 may also include an implanted rechargeable battery for energizing energy consuming implanted components of the device. In this case, the implanted energy-transforming device 111A also charges the battery with electric energy, as the energy-transforming device transforms energy from the signal into the electric energy. A reversing device in the form of an electric switch 114, such as a microprocessor, is implanted in the patient for reversing the constriction device of the constriction/stimulation unit 110. The wireless remote control of the external energy-transmission device 113 transmits a wireless signal that carries energy and the implanted energy-transforming device 111A transforms the wireless energy into a current for operating the switch 114. When the polarity of the current is shifted by the energy-transforming-device 111A the switch 114 reverses the function performed by the constriction device of the constriction/stimulation unit 110. FIG. 520 shows an embodiment including the energy-transforming device 111A, the constriction/stimulation unit 110 and an implanted operation device in the form of a motor 115 for operating the constriction device of the constriction/stimulation unit 110. The motor 115 is powered with energy from the energy-transforming device 111A, as the remote control of the external energy-transmission device 113 transmits a wireless signal to the receiver of the energy-transforming device 111A. FIG. 521 shows an embodiment including the energy-transforming device 111A, the constriction/stimulation unit 110 and an implanted assembly 116 including a motor/pump unit 117 and a fluid reservoir 118. In this case the constriction device of the constriction/stimulation unit 110 is hydraulically operated, i.e., hydraulic fluid is pumped by the motor/pump unit 117 from the reservoir 118 to the constriction/stimulation unit 110 to constrict the wall portion, and hydraulic fluid is pumped by the motor/pump unit 117 back from the constriction/stimulation unit 110 to the reservoir 118 to release the wall portion. The implanted energy-transforming device 111A transforms wireless energy into a current, for powering the motor/pump unit 117. FIG. 522 shows an embodiment comprising the external energy-transmission device 113 that controls the control unit 102 to reverse the motor 115 when needed, the constriction/stimulation unit 110, the constriction device of which is hydraulically operated, and the implanted energy-transforming device 111A, and further comprising an implanted hydraulic fluid reservoir 119, an implanted motor/pump unit 120, an implanted reversing device in the form of a hydraulic valve shifting device 121 and a separate external wireless remote control 111B. The motor of the motor/pump unit 120 is an electric motor. In response to a control signal from the wireless remote control of the external energy-transmission device 113, the implanted energy-transforming device 111A powers the motor/pump unit 120 with energy from the energy carried by the control signal, whereby the motor/pump unit 120 distributes hydraulic fluid between the reservoir 119 and the constriction device of the constriction/stimulation unit 110. The remote control 111B controls the shifting device 121 to shift the hydraulic fluid flow direction between one direction in which the fluid is pumped by the motor/pump unit 120 from the reservoir 119 to the constriction device of the constriction/stimulation unit 110 to constrict the wall portion, and another opposite direction in which the fluid is pumped by the motor/pump unit 120 back from the constriction device of the constriction/stimulation unit 110 to the reservoir 119 to release the wall portion. FIG. 523 shows an embodiment including the energy-transforming device 111A and the constriction/stimulation unit 110. A control unit 102, an accumulator 123 and a capacitor 124 are also implanted in the patient. A separate external wireless remote control 111B controls the control unit 102. The control unit 102 controls the energy-transforming device 111A to store electric energy in the accumulator 123, which supplies energy to the constriction/stimulation unit 110. In response to a control signal from the wireless remote control 111B, the control unit 102 either releases electric energy from the accumulator 123 and transfers the released energy via power lines, or directly transfers electric energy from the energy-transforming device 111A via the capacitor 124, which stabilizes the electric current, for the operation of the constriction/stimulation unit 110. In accordance with one alternative, the capacitor 124 in the embodiment of FIG. 523 may be omitted. In accordance with another alternative, the accumulator 123 in this embodiment may be omitted. FIG. 524 shows an embodiment including the energy-transforming device 111A, the constriction/stimulation unit 110. A battery 125 for supplying energy for the operation of the constriction/stimulation unit 110 and an electric switch 126 for switching the operation of the constriction/stimulation unit 110 are also implanted in the patient. The switch 126 is operated by the energy supplied by the energy-transforming device 111A to switch from an off mode, in which the battery 125 is not in use, to an on mode, in which the battery 125 supplies energy for the operation of the constriction/stimulation unit 110. FIG. 525 shows an embodiment identical to that of FIG. 524, except that a control unit 102 also is implanted in the patient. A separate external wireless remote control 111B controls the control unit 102. In this case, the switch 126 is operated by the energy supplied by the energy-transforming device 111A to switch from an off mode, in which the wireless remote control 111B is prevented from controlling the control unit 102 and the battery 125 is not in use, to a standby mode, in which the remote control 111B is permitted to control the control unit 102 to release electric energy from the battery 125 for the operation of the constriction/stimulation unit 110. FIG. 525 shows an embodiment identical to that of FIG. 525, except that the accumulator 123 is substituted for the battery 125 and the implanted components are interconnected differently. In this case, the accumulator 123 stores energy from the energy-transforming device 111A. In response to a control signal from the wireless remote control 111B, the implanted control unit 102 controls the switch 126 to switch from an off mode, in which the accumulator 123 is not in use, to an on mode, in which the accumulator 123 supplies energy for the operation of the constriction/stimulation unit 110. FIG. 526 shows an embodiment identical to that of FIG. 525, except that the battery 125 also is implanted in the patient, and the implanted components are interconnected differently. In response to a control signal from the wireless remote control 111B, the implanted control unit 102 controls the accumulator 123, which may be a capacitor, to deliver energy for operating the switch 126 to switch from an off mode, in which the battery 125 is not in use, to an on mode, in which the battery 125 supplies electric energy for the operation of the constriction/stimulation unit 110. Alternatively, the switch 126 may be operated by energy supplied by the accumulator 123 to switch from an off mode, in which the wireless remote control 111B is prevented from controlling the battery 125 to supply electric energy and the battery 125 is not in use, to a standby mode, in which the wireless remote control 111B is permitted to control the battery 125 to supply electric energy for the operation of the constriction/stimulation unit 110. FIG. 527 shows an embodiment identical to that of FIG. 524, except that a motor 115, a mechanical reversing device in the form of a gearbox 127 and a control unit 102 for controlling the gearbox 127 also are implanted in the patient. A separate external wireless remote control 111B controls the implanted control unit 102 to control the gearbox 127 to reverse the function performed by the constriction device (mechanically operated) of the constriction/stimulation unit 110. FIG. 529 shows an embodiment identical to that of FIG. 526, except that the implanted components are interconnected differently. Thus, in this case, the battery 125 powers the control unit 102 when the accumulator 123, suitably a capacitor, activates the switch 126 to switch to an on mode. When the switch 126 is in its on mode the control unit 102 is permitted to control the battery 125 to supply, or not supply, energy for the operation of the constriction/stimulation unit 110. FIG. 529 shows an embodiment identical to that of FIG. 520, except that a gearbox 127 that connects the motor 115 to the constriction/stimulation unit 110, and a control unit 102 that controls the energy-transforming device 111A to power the motor 115 also are implanted in the patient. There is a separate external wireless remote control 111B that controls the control unit 102 to reverse the motor 115 when needed. Optionally, the accumulator 123 shown in FIG. 523 may be provided in the embodiment of FIG. 529, wherein the implanted control unit 102 controls the energy-transforming device 111A to store the transformed energy in the accumulator 123. In response to a control signal from the wireless remote control 111B, the control unit 102 controls the accumulator 123 to supply energy for the operation of the constriction/stimulation unit 110. Those skilled in the art will realize that the above various embodiments according to FIGS. 519-529 could be combined in many different ways. For example, the energy operated switch 114 could be incorporated in any of the embodiments of FIGS. 520, 523-529, the hydraulic shifting device 121 could be incorporated in the embodiment of FIG. 523, and the gearbox 127 could be incorporated in the embodiment of FIG. 520. The switch 114 may be of a type that includes electronic components, for example a microprocessor, or a FGPA (Field Programmable Gate Array) designed for switching. Alternatively, however, the energy operated switch 114 may be replaced by a subcutaneously implanted push button that is manually switched by the patient between “on” and “off”. Alternatively, a permanent or rechargeable battery may be substituted for the energy-transforming devices 111A of the embodiments shown in FIGS. 518-529. FIG. 531 shows basic parts of a remote control of the device for controlling the constriction/stimulation unit 110. In this case, the stimulation device of the constriction/stimulation unit stimulates the wall portion with electric pulses. The remote control is based on wireless transmission of electromagnetic wave signals, often of high frequencies in the order of 100 kHz-1 GHz, through the skin 132 of the patient. In FIG. 531, all parts placed to the left of the skin 132 are located outside the patient's body and all parts placed to the right of the skin 132 are implanted.

An external signal-transmission device 133 is to be positioned close to a signal-receiving device 134 implanted close to the skin 132. As an alternative, the signal-receiving device 134 may be placed for example inside the abdomen of the patient. The signal-receiving device 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The signal transmission device 133 comprises a coil having about the same size as the coil of the signal-receiving device 134 but wound with a thick wire that can handle the larger currents that is necessary. The coil of the signal transmission device 133 is tuned to the same specific high frequency as the coil of the signal-receiving device 134.

The signal-transmission device 133 is adapted to send digital information via the power amplifier and signal-receiving device 134 to an implanted control unit 135. To avoid that accidental random high frequency fields trigger control commands, digital signal codes are used. A conventional keypad placed on the signal transmission device 133 is used to order the signal transmission device 133 to send digital signals for the control of the constriction/stimulation unit. The signal transmission device 133 starts a command by generating a high frequency signal. After a short time, when the signal has energized the implanted parts of the control device, commands are sent to operate the constriction device of the constriction/stimulation unit 110 in predefined steps. The commands are sent as digital packets in the form illustrated below.
Start pattern, 8 bits
Command, 8 bits
Count, 8 bits
Checksum, 8 bits

The commands are sent continuously during a rather long time period (e.g., about 30 seconds or more). When a new constriction or release step is desired, the Count byte is increased by one to allow the implanted control unit 135 to decode and understand that another step is demanded by the signal transmission device 133. If any part of the digital packet is erroneous, its content is simply ignored.

Through a line 136, an implanted energizer unit 137 draws energy from the high frequency electromagnetic wave signals received by the signal-receiving device 134. The energizer unit 137 stores the energy in a source of energy, such as a large capacitor, powers the control unit 135 and powers the constriction/stimulation unit 110 via a line 138.

The control unit 135 comprises a demodulator and a microprocessor. The demodulator demodulates digital signals sent from the signal transmission device 133. The microprocessor receives the digital packet, decodes it, and sends a control signal via a signal line 139 to control the constriction device of the constriction/stimulation unit 110 to either constrict or release the wall portion of the patient's organ depending on the received command code.

FIG. 532 shows a circuitry of an embodiment of the invention, in which wireless energy is transformed into a current. External components of the circuitry include a microprocessor 140, a signal generator 141 and a power amplifier 142 connected thereto. The microprocessor 140 is adapted to switch the signal generator 141 on/off and to modulate signals generated by the signal generator 141 with digital commands. The power amplifier 142 amplifies the signals and sends them to an external signal-transmitting antenna coil 143. The antenna coil 143 is connected in parallel with a capacitor 144 to form a resonant circuit tuned to the frequency generated by the signal generator 141.

Implanted components of the circuitry include a signal receiving antenna coil 145 and a capacitor 146 forming together a resonant circuit that is tuned to the same frequency as the transmitting antenna coil 143. The signal receiving antenna coil 145 induces a current from the received high frequency electromagnetic waves and a rectifying diode 147 rectifies the induced current, which charges a storage capacitor 148. The storage capacitor 148 powers a motor 149 for driving the constriction device of the constriction/stimulation unit 110. A coil 150 connected between the antenna coil 145 and the diode 147 prevents the capacitor 148 and the diode 147 from loading the circuit of the signal-receiving antenna 145 at higher frequencies. Thus, the coil 150 makes it possible to charge the capacitor 148 and to transmit digital information using amplitude modulation.

A capacitor 151 and a resistor 152 connected in parallel and a diode 153 form a detector used to detect amplitude modulated digital information. A filter circuit is formed by a resistor 154 connected in series with a resistor 155 connected in series with a capacitor 156 connected in series with the resistor 154 via ground, and a capacitor 157, one terminal of which is connected between the resistors 154, 155 and the other terminal of which is connected between the diode 153 and the circuit formed by the capacitor 151 and resistor 152. The filter circuit is used to filter out undesired low and high frequencies.

The detected and filtered signals are fed to an implanted microprocessor 158 that decodes the digital information and controls the motor 149 via an H-bridge 159 comprising transistors 160, 161, 162 and 163. The motor 149 can be driven in two opposite directions by the H-bridge 159.

The microprocessor 158 also monitors the amount of stored energy in the storage capacitor 148. Before sending signals to activate the motor 149, the microprocessor 158 checks whether the energy stored in the storage capacitor 148 is enough. If the stored energy is not enough to perform the requested operation, the microprocessor 158 waits for the received signals to charge the storage capacitor 148 before activating the motor 149.

Alternatively, the energy stored in the storage capacitor 148 may only be used for powering a switch, and the energy for powering the motor 149 may be obtained from another implanted energy source of relatively high capacity, for example a battery. In this case the switch is adapted to connect the battery to the motor 149 in an on mode when the switch is powered by the storage capacitor 148 and to keep the battery disconnected from the motor 149 in a standby mode when the switch is not powered.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

### Q: Controlling Movement of Sperms

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for controlling the movement of sperms in the uterine tubes of a female patient, examples of such devices for controlling the movement of sperms in the uterine tubes of a female patient will now be described.

Referring to the drawing figures, like reference numerals designate identical or corresponding elements throughout the several figures.

FIGS. 533A-533c schematically illustrate different states of operation of a generally designed apparatus according to the present invention, when the apparatus is applied on a wall portion of a uterine tube designated BO. The apparatus includes a constriction device and a stimulation device, which are designated CSD, and a control device designated CD for controlling the constriction and stimulation devices CSD. FIG. 533A shows the apparatus in an inactivation state, in which the constriction device does not constrict the uterine tube BO and the stimulation device does not stimulate the uterine tube BO.

FIG. 533B shows the apparatus in a constriction state, in which the control device CD controls the constriction device to gently constrict the wall portion of the uterine tube BO to a constricted state, in which the blood circulation in the constricted wall portion is substantially unrestricted and the flow in the uterine tube of the wall portion is restricted. FIG. 533C shows the apparatus in a stimulation state, in which the control device CD controls the stimulation device to stimulate different areas of the constricted wall portion, so that almost the entire wall portion of the uterine tube BO contracts (thickens) and closes the uterine tube.

FIGS. 533D and 533E show how the stimulation of the constricted wall portion can be cyclically varied between a first stimulation mode, in which the left area of the wall portion (see FIG. 533D) is stimulated, while the right area of the wall portion is not stimulated, and a second stimulation mode, in which the right area of the wall portion (see FIG. 533E) is stimulated, while the left area of the wall portion is not stimulated, in order to maintain over time satisfactory blood circulation in the constricted wall portion.

It should be noted that the stimulation modes shown in FIGS. 533D and 1E only constitute a principle example of how the constricted wall portion of the uterine tube BO may be stimulated. Thus, more than two different areas of the constricted wall portion may be simultaneously stimulated in cycles or successively stimulated. Also, groups of different areas of the constricted wall portion may be successively stimulated.

FIGS. 533F, 533G and 533H illustrate different states of operation of a modification of the general embodiment shown in FIGS. 1A-1E, wherein the constriction and stimulation devices CSD include several separate constriction/stimulation elements, here three elements CSDE1, CSDE2 and CSDE3. FIG. 533F shows how the element CSDE1 in a first state of operation is activated to both constrict and stimulate the uterine tube BO, so that the uterine tube of the uterine tube BO is closed, whereas the other two elements CSDE2 and CSDE3 are inactivated. FIG. 533G shows how the element CSDE2 in a second following state of operation is activated, so that the uterine tube of the uterine tube BO is closed, whereas the other two elements CSDE1 and CSDE3 are inactivated. FIG. 533H shows how the element CSDE3 in a following third state of operation is activated, so that the uterine tube of the uterine tube BO is closed, whereas the other two elements CSDE1 and CSDE2 are inactivated. By shifting between the first, second and third states of operation, either randomly or in accordance with a predetermined sequence, different portions of the uterine tube can by temporarily constricted and stimulated while maintaining the uterine tube closed, whereby the risk of injuring the uterine tube is minimized. It is also possible to activate the elements CSDE1-CSDE3 successively along the uterine tube to move sperms in the uterine tube. FIGS. 5331, 533K and 533L illustrate an alternative mode of operation of the modification of the general embodiment. Thus, FIG. 533I shows how the element CSDE1 in a first state of operation is activated to both constrict and stimulate the uterine tube BO, so that the uterine tube BO is closed, whereas the other two elements CSDE2 and CSDE3 are activated to constrict but not stimulate the uterine tube BO, so that the uterine tube BO is not completely closed where the elements CSDE2 and CSDE3 engage the uterine tube BO. FIG. 533K shows how the element CSDE2 in a second following state of operation is activated to both constrict and stimulate the uterine tube BO, so that the uterine tube BO is closed, whereas the other two elements CSDE1 and CSDE3 are activated to constrict but not stimulate the organ BO, so that the uterine tube BO is not completely closed where the elements CSDE1 and CSDE3 engage the uterine tube BO. FIG. 533L shows how the element CSDE3 in a following third state of operation is activated to both constrict and stimulate the uterine tube BO, so that the uterine tube BO is closed, whereas the other two elements CSDE1 and CSDE2 are activated to constrict but not stimulate the uterine tube BO, so that the uterine tube BO is not completely closed where the elements CSDE1 and CSDE2 engage the uterine tube BO. By shifting between the first, second and third states of operation, either randomly or in accordance with a predetermined sequence, different portions of the uterine tube can by temporarily stimulated while maintaining the uterine tube closed, whereby the risk of injuring the uterine tube is reduced. It is also possible to activate the stimulation of the elements CSDE1-CSDE3 successively along the uterine tube BO to move fluids and/or other bodily matter in the uterine tube. FIGS. 534-536 show basic components of an embodiment of the apparatus according to the invention for controlling a flow of sperms in a uterine tube formed by a uterine tube wall of a patient's uterine tube. The apparatus comprises a tubular housing 1 with open ends, a constriction device 2 arranged in the housing 1, a stimulation device 3 integrated in the constriction device 2, and a control device 4 (indicated in FIG. 536) for controlling the constriction and stimulation devices 2 and 3. The constriction device 2 has two elongate clamping elements 5, 6, which are radially movable in the tubular housing 1 towards and away from each other between retracted positions, see FIG. 534, and clamping positions, see FIG. 536. The stimulation device 3 includes a multiplicity of electrical elements 7 positioned on the clamping elements 5, 6, so that the electrical elements 7 on one of the clamping elements 5, 6 face the electrical elements 7 on the other clamping element. Thus, in this embodiment the constriction and stimulation devices form a constriction/stimulation unit, in which the constriction and stimulation devices are integrated in a single piece. The constriction and stimulation devices may also be separate from each other. In this case, a structure may be provided for holding the electrical elements 7 in a fixed orientation relative to one another. Alternatively, the electrical elements 7 may include electrodes that are separately attached to the wall portion of the patient's uterine tube. FIGS. 537A-537C illustrate in principle the function of the apparatus of FIG. 535 when the apparatus is applied on a portion 8 of a tubular uterine tube wall of a patient's uterine tube. Thus, FIG. 537A shows the apparatus in a non-clamping state, in which the clamping elements 5, 6 are in their retracted positions and the wall portion 8 extends through the open ends of the housing 1 without being constricted by the clamping elements 5, 6. FIG. 537B shows the apparatus in a clamping state, in which the clamping elements 5, 6 have been moved from their retracted positions to their clamping positions, in which the clamping elements 5, 6 gently constrict the wall portion 8 to a constricted state, in which the blood circulation in the constricted wall portion 8 is substantially unrestricted and the flow in the uterine tube of the wall portion 8 is restricted. FIG. 537C shows the apparatus in a stimulation state, in which the clamping elements 5, 6 constrict the wall portion 8 and the electrical elements 7 of the stimulation device 3 electrically stimulate different areas of the wall portion 8, so that the wall portion 8 contracts (thickens) and closes the uterine tube. When the apparatus is in its stimulation state, it is important to stimulate the different areas of the wall portion 8 in a manner so that they essentially maintain their natural physical properties over time to prevent the areas from being injured. Consequently, the control device 4 controls the stimulation device 3 to intermittently stimulate each area of the wall portion 8 during successive time periods, wherein each time period is short enough to maintain over time satisfactory blood circulation in the area. Furthermore, the control device 4 controls the stimulation of the areas of the wall portion 8, so that each area that currently is not stimulated restores substantially normal blood circulation before it is stimulated again. To maintain over time the effect of stimulation, i.e., to keep the uterine tube closed by maintaining the wall portion 8 contracted, the control device 4 controls the stimulation device 3 to stimulate one or more of the areas at a time and to shift the stimulation from one area to another over time. The control device 4 may control the stimulation device 3 to cyclically propagate the stimulation of the areas along the tubular wall portion 8, for example, in accordance with a determined stimulation pattern. To achieve the desired reaction of the uterine tube wall during the stimulation thereof, the control device may control the stimulation device to, preferably cyclically, vary the intensity of the stimulation of the wall portion 8. In the embodiment of FIGS. 534-536, the electrical elements 7 form a series of fourteen groups of electrical elements 7 extending longitudinally along each elongate clamping element 5 and 6, respectively, see FIG. 534. The electrical elements 7 of each group of electrical elements 7 form a first path of four electrical elements 7 positioned in a row on clamping element 5 and extending transverse thereto, and a second path of four electrical elements 7 positioned in a row on clamping element 6 and extending transverse thereto. Thus, the two paths of electrical elements 7 extend on mutual sides of the patient's uterine tube. The control device 4 controls the stimulation device 3 to successively energize the groups of electrical elements 7 in the series of groups in a direction opposite to or, alternatively, in the same direction as that of the flow in the uterine tube of the patient's uterine tube. Of course, the number of electrical elements 7 of each path of electrical elements 7 can be greater or smaller than four, and several parallel rows electrical elements 7 can form each path of electrical elements 7.

FIGS. 538A-538C show another embodiment which includes a tubular housing 9 and three elongate clamping elements 10a, 10b, 10c, which are radially movable in the tubular housing 9 towards and away from a central axis thereof between retracted positions, see FIG. 538A, and clamping positions, see FIG. 538B. The three clamping elements 10a-10c are symmetrically disposed around the central axis of the housing 9. The stimulation device of this embodiment includes electrical elements 11a, 11b, 11c that form a series of groups of elements extending longitudinally along the elongate clamping elements 10a-10c, wherein the electrical elements 11a-11c of each group of electrical elements form a path of three electrical elements 11a, 11b and 11c extending circumferentially around the central axis of the housing 9. The three electrical elements 11a-11c of each group are positioned on the three clamping elements 10a-10c, respectively. Thus, the path of three electrical elements 11a-11c extends around the patient's uterine tube. Of course, the number of electrical elements 11a-11c of each path of electrical elements can be greater than three, and several parallel rows electrical elements 11a-11c can form each path of electrical elements. FIGS. 539A and 539B show different steps of an electric stimulation mode performed by the apparatus of FIG. 534 while the clamping elements 5, 6 of the apparatus are constricting a portion of a tubular uterine tube wall of a patient's uterine tube 12 to restrict the flow in the uterine tube 13 of the organ 12. For the sake of clarity only the clamping elements 5, 6 of the constriction device 2 are shown in FIGS. 539A, 539B. Thus, FIG. 539A illustrates how energized electrical elements 7 of groups of electrical elements electrically stimulate a first portion 14 and a second portion 15 of the tubular wall to contract and close the uterine tube 13. FIG. 539B illustrates how energized electrical elements 7 of other groups of electrical elements electrically stimulate a third portion 16 of the tubular wall different from the first and second portions to contract and close the uterine tube 13, while the electrical stimulation of the first and second portions 14, 15 of the tubular wall has been ceased, so that substantially normal blood circulation in the first and second portions is restored. In this manner, the electric stimulation of the constricted tubular wall is shifted over time from one portion of the tubular wall to another to insure recurrent restoration of blood circulation in the constricted tubular wall. The control device 4 controls the stimulation device 3 to energize the electrical elements 7 with electric biphasic pulses, i.e., combined positive and negative pulses. The desired stimulation effect is achieved by varying different pulse parameters. Thus, the control device 4 controls the stimulation device 3 to vary the pulse amplitude (voltage), the off time period between successive pulses, the pulse duration and the pulse repetition frequency. The pulse current should be between 1 to 30 mA. For neural stimulation, a pulse current of about 5 mA and a pulse duration of about 300 s are suitable, whereas a pulse current of about 20 mA and a pulse duration of about 30 s are suitable for muscular stimulation. The pulse repetition frequency suitably is about 10 Hz. For example, as illustrated in the Pulse/time diagram P/t of FIG. 540A, a pulse combination including a negative pulse PS of short duration and high amplitude (voltage), and a positive pulse PL of long duration and low amplitude following the negative pulse may be cyclically repeated to form a pulse train of such pulse combinations. The energy content of the negative pulse PS should be substantially equal to the energy content of the positive pulse PL. FIG. 540B is a pulse/time diagram showing a modification of the electric stimulation shown in FIG. 540A. Thus, the pulse combination of FIG. 540A is mixed with a pulse train combination having a first relatively long pulse train PTL of high frequency/low amplitude pulses, appearing simultaneously with the positive pulse PL of the pulse combination of FIG. 540A, and a second relatively short pulse train PTS of high frequency/low amplitude appearing simultaneously with the negative pulse PS of the pulse combination shown in FIG. 540A. As a result, the high frequency/low amplitudes pulse trains PTL and PTS are superimposed on the positive and negative pulses PL and PS of FIG. 540A, as illustrated in FIG. 540B. The pulse configuration of FIG. 540B, and variations thereof, is beneficial to use in connection with the stimulation of the uterine tube, in order to achieve the desired stimulation effect. Preferably, the electric pulses form pulse trains, as illustrated in the Pulse/time diagrams P/t of FIGS. 541A-542B. The Pulse/time diagram P/t of FIG. 541A represents an individual area of the wall portion of the patient's uterine tube which is stimulated with a pulse train 18A. The pulse train 18A includes three initial negative pulses, each of which is of short duration and high amplitude (voltage), and one positive pulse of long duration and low amplitude following the negative pulses. After a delay to enable the area of the uterine tube to restore substantially normal blood circulation, the pulse train 18A is repeated. The Pulse/time diagram P/t of FIG. 541B represents another individual area of the wall portion, which is stimulated with a pulse train 18B having the same configuration as the pulse train 18A. The pulse trains 18A and 18B are shifted relative to each other, so that they partially overlap one another to ensure that the constricted wall portion always is stimulated to contract as desired. The pulse/time diagrams P/t of FIGS. 541A and 541B represent two different areas of the wall portion, which are stimulated with cyclically repeated pulse trains 18C and 18D, respectively, having the same configuration. Each pulse train 18C, 18D includes two initial negative pulses, each of which is of short duration and high amplitude (voltage), and one positive pulse of long duration and low amplitude following the two negative pulses. In this case, the pulse trains 18C and 18D are shifted relative to each other, so that they do not overlap each other. Thus, the off time period between adjacent pulse trains 18C is longer than the duration of pulse train 18D and the off time period between adjacent pulse trains 18D is longer than the duration of pulse train 18C. The pulse trains 18A, 18B, 18C and 18D can be configured in many different ways. Thus, the control device 4 can control the stimulation device 3 to vary the length of each pulse train, the repetition frequency of the pulse trains, the number of pulses of each pulse train, and/or the off time periods between the pulse trains. Typically, the control device 4 controls each off time period between the pulse trains to last long enough to restore substantially normal blood circulation in the area that just has been stimulated before that area again is stimulated with electric pulses. FIGS. 543A and 543B show another embodiment that controls sperm flow in a uterine tube 19, comprising a constriction device with two clamping elements 20a and 20b, a stimulation device in the form of two thermal stimulation elements 21a and 21b integrated in the clamping elements 20a, 20b, respectively, and a control device 4 for controlling the clamping elements 20a, 20b and stimulation elements 21a, 21b. The clamping elements 20a and 20b are movable towards and away from each other in the same manner as described above in connection with the embodiment according to FIGS. Q5A-Q5C. The thermal stimulation elements 21a and 21b, which may include Pertier elements, are positioned on the clamping elements 20a, 20b, so that the thermal elements 21a are facing the thermal elements 21b. FIG. 543A shows how the clamping elements 20a, 20b constrict the uterine tube 19, so that the sperm flow is restricted. FIG. 543B shows how the control device 4 controls the thermal stimulation elements 21a, 21b to cool the wall of the uterine tube 19, so that the wall contracts and closes the uterine tube 19. To release the uterine tube 19, the control device 4 controls the thermal stimulation elements 21a, 21b to heat the wall of the uterine tube 19, so that the wall expands. FIGS. 544A and 544B show hydraulic operation means suited for operating the constriction device of the embodiments described above. Specifically, FIGS. 544A and 544B show the apparatus of FIG. 534 provided with such means for hydraulic operation of the constriction device 2. (The stimulation device is not shown.) Thus, the housing 1 forms two hydraulic chambers 22a and 22b, in which the two clamping elements 5, 6 are slidable back and forth relative to the tubular uterine tube wall portion 8 of a patient's uterine tubes. The hydraulic operation means include an expandable reservoir 23, such as an elastic balloon, containing hydraulic fluid, conduits 24a and 24b between the reservoir 23 and the hydraulic chambers 22a, 22b, and a two-way pump 25 for pumping the hydraulic fluid in the conduits 24a, 24b. The control device 4 controls the pump 25 to pump hydraulic fluid from the reservoir 23 to the chambers 22a, 22b to move the clamping elements 5, 6 against the wall portion 8, whereby the wall portion 8 is constricted, see FIG. 544B, and to pump hydraulic fluid from the chambers 22a, 22b to the reservoir 23 to move the clamping elements 5, 6 away from the wall portion 8, whereby the tubular wall 8 is released, see FIG. 544A. Alternatively, the embodiment of FIGS. 544A and 544B may be manually operated by applying suitable manually operable hydraulic means for distributing the hydraulic fluid between the expandable reservoir 23 and the hydraulic chambers 22a, 22b. In this case the pump 25 is omitted. FIGS. 545A and 545B schematically show a mechanically operable embodiment of the invention, comprising an open ended tubular housing 26 applied on the tubular uterine tube wall portion 8 of a patient's uterine tube, a constriction device 27 arranged in the housing 26 and a control device 4 for controlling the constriction device 27. A stimulation device (not shown) as described above is also provided in the housing 26. The constriction device 27 includes a clamping element 28, which is radially movable in the tubular housing 26 towards and away from the tubular wall portion 8 between a retracted position, see FIG. 545A, and a clamping position, see FIG. 545B, in which the clamping element 28 gently constricts the tubular wall portion 8. Mechanical operation means for mechanically operating the clamping element 28 includes an electric motor 29 attached to the housing 26 and a telescopic device 30, which is driven by the motor 29 and operatively connected to the clamping element 28. The control device 4 controls the electric motor 29 to expand the telescopic device 30 to move the clamping element 28 against the wall portion 8, whereby the tubular wall portion 8 is constricted, see FIG. 545B, and controls the motor 29 to retract the telescopic device 30 to move the clamping element 28 away from the wall portion 8, whereby the wall portion 8 is released, see FIG. 545A. Alternatively, the motor 29 may be omitted and the telescopic device 30 be modified for manual operation, as shown in FIG. 545C. Thus, a spring 30a may be provided acting to keep the telescopic device 30 expanded to force the clamping element 28 against the wall portion 8. The mechanical operation means may include a subcutaneously implanted lever mechanism 29a that is operatively connected to the telescopic device 30. The patient may push the lever mechanism 29a through the patient's skin 29b to pull the telescopic device 30 against the action of the spring 30a to the retracted position of the telescopic device 30, as indicated in phantom lines. When the patient releases the lever mechanism 29a, the spring 30a expands the telescopic device 30, whereby clamping element 28 is forced against the wall portion 8. The mechanical operation means as described above in connection with FIGS. 545A, 545B and 545C may also be implemented in the embodiments according to FIGS. 533-543. FIG. 545A illustrates the embodiment of FIG. 534 applied on the uterine tube of a patient. The clamping elements 5, 6 of the constriction device 2 constrict the uterine tubes 31 and the stimulation device 3 is energized to close the uterine tube. (For the sake of clarity, the housing is not shown and the clamping elements 5, 6 are exaggerated.) In this embodiment, a control device includes an external control unit in the form of a hand-held wireless remote control 32, and an implanted internal control unit 33, which may include a microprocessor, for controlling the constriction and stimulation devices. The remote control 32 is operable by the patient to control the internal control unit 33 to switch on and off the constriction device and/or the stimulation device. Alternatively, however, the remote control 32 may be replaced by a subcutaneously implanted push button that is manually switched by the patient between “on” and “off”. Such a manually operable push button may also be provided in combination with the remote control 32 as an emergency button to allow the patient to stop the operation of the apparatus in case of emergency or malfunction. The internal control unit 33 controls an implanted operation device 34 to move the clamping elements 5, 6. An implanted source of energy 35, such as a rechargeable battery, powers the operation device 34. The internal control unit 33, which may be implanted subcutaneously or in the abdomen, also works as an energy receiver, i.e., for transforming wireless energy into electric energy and charging the implanted source of energy 35 (rechargeable battery) with the electric energy. An implanted sensor 36 senses a physical parameter of the patient, such as the pressure in the uterine tubes, or a parameter that relates to the pressure in the uterine tubes, wherein the internal control unit 33 controls the constriction device 2 and/or the stimulation device 3 in response to signals from the sensor 36. In this embodiment the sensor 36 is a pressure sensor, wherein the internal control unit 33 controls the constriction device and/or stimulation device to change the constriction of the patient's intestines 31 in response to the pressure sensor 36 sensing a predetermined value of measured pressure. For example, the control unit 33 may control the constriction device and/or stimulation device to increase the constriction of the patient's uterine tubes 31 in response to the pressure sensor sensing an increased pressure. Alternatively or in combination, the remote control 32 controls the constriction device and/or stimulation device in response to signals from the sensor 36, in the same manner as the internal control unit 33. The remote control 32 may be equipped with means for producing an indication, such as a sound signal or displayed information, in response to signals from the sensor 36. When the patient's attention is taken by such an indication indicating an increased pressure exceeding a threshold value, he or she may use the remote control to control the constriction device and stimulation device to pump sperms through the patient's uterine tubes towards the ovary. If pregnancy wants to be achieved or pump in the opposite direction to avoid pregnancy. Of course, the constriction device 2 shown in FIG. 545 may be replaced by any one of the constriction devices described in the various embodiments of the present invention, where applicable. FIGS. 546-548 show a mechanically operable constriction device having an elongated constriction member in the form of a circular resilient core 37 with two overlapping end portions 38, 39. The core 37 defines a substantially circular restriction opening and is enclosed in an elastic soft hose 40 except at a releasable and lockable joint 41 of the core 37, which when released enables application of the core 37 with its hose 40 around a portion of a tubular uterine tube wall of a patient's uterine tubes. The materials of all of these elements are bio-compatible so that the patient' body will not reject them. An operation device 34 for mechanically operating the longitudinal extension of the core 37 to change the size of the restriction opening comprises a drive wheel 43 in frictional engagement with the overlapping end portions 38, 39 of the core 37. The drive wheel 43 is journaled on a holder 44 placed in the hose 40 and provided with two counter pressure rollers 45, 46 pressing the respective end portions 38, 39 of the core 37 against the drive wheel 43 to increase the frictional engagement there between. An electric motor 47 of the operation device is connected to the drive wheel 43 via a long flexible drive shaft 48, and is moulded together with a remote controlled power supply unit 49 in a body 50 of silicone rubber. The length of the flexible drive shaft 48 is selected so that the body 50 can be placed in a desired position in the patient's body, suitably in the abdomen. The power supply unit 49 can be controlled to power the electric motor 47 to turn the drive wheel 43 in one direction to reduce the diameter of the core 37, so that the wall portion is constricted, or to turn the drive wheel 43 in the opposite direction to increase the diameter of the core 37, so that the wall portion is released. In accordance with a first alternative, a rack gear may be formed on one of the end portions 38, 39 of the core 37 and the drive wheel 43 may be replaced by a drive gear wheel connected to the other end portion of the core 37 and in mesh with the rack gear. In accordance with a second alternative, the operation device 42 may be designed as a worm-driven hose clamp, i.e., one of the end portions 38, 39 of the core 37 may be provided with threads and the other end portion of the core 37 may be provided with a worm, the threads of which interacts with the threads of the one end portion of the core 37. The threads of such a worm may also interact with threads provided on both end portions 38, 39 of the core 37. In this alternative, the electric motor 47 turns the worm in one direction to reduce the diameter of the core 37, so that the wall portion is constricted, or turn the worm in the opposite direction to increase the diameter of the core 37, so that the wall portion is released in one direction to reduce the diameter of the core 37, so that the wall portion is constricted, or turns the clamping screw in the opposite direction to increase the diameter of the core 37, so that the wall portion is released. FIG. 549 shows a constriction device which is identical to the embodiment of FIGS. 546-548, except that the motor 47 is encapsulated in the hose 40 so that it is fixed to the core 37 and has a short drive shaft 51, and that the motor 47 is positioned relative to the core 37, such that the drive shaft 51 extends substantially tangentially to the circular core 37. There is an angular gearing 52 connecting the drive shaft 51 to the drive wheel 43. FIG. 550 shows a suitable alternative arrangement for the motor 47 in the embodiment of FIG. 549, comprising a first clamping member 53 secured to one end portion of the core 37 and a second clamping member 54 secured to the other end portion 39 of the core 37. The motor 47 is secured to the first clamping member 53 and is operatively connected to a worm gear 55 via a gear transmission 56. The worm gear 55 is journaled at its opposite ends on holders 57 and 58, which are rigidly secured to the clamping member 53 and the motor 47, respectively. The second clamping member 54 has a pinion in mesh with the worm gear 55. When the motor 47 is powered, the worm gear 55 rotates, and will thereby pull the end portion 39 of the core 37 in one or the opposite longitudinal direction, so that the diameter of the substantially circular core 37 is either increased or decreased. The motor 47, worm gear 55, gear transmission 56 and second clamping member 54 constitute a servo system of the type that transfers a weak force acting on a moving element having a long stroke into a strong force acting on another moving element having a short stroke. FIG. 552 shows a constriction device including a plurality of arcuate lamellae 59 arranged like the conventional adjustable aperture mechanism of a camera. A motor 60 operates the lamellae 59 to change the size of a restriction opening defined by the lamellae 59. FIGS. 122-124 show a constriction device including two semi-circular elements 61 and 62, which are hinged together such that the semi-circular elements 61, 62 are swingable relative to each other between a fully open state in which they substantially form a circle, as illustrated in FIG. 122, and an angular state, in which the size of the restriction opening defined by the semi-circular elements 61, 62 is reduced, as illustrated in FIG. 554. A motor 63 operates the semi-circular elements 61, 62 to swing them relative to each other. FIGS. 555-557 show a constriction device including an elastic belt 64 forming a circle and having a substantially oval cross-section. A motor 67 operates the belt 64 to turn around the longitudinal extension thereof between a fully open state, in which the inner broader side of the belt 64 forms a substantially cylindrical surface, as illustrated in FIG. 556, and a reduced open state, in which the inner broader side of the belt 64 forms a substantially conical surface, as illustrated in FIG. 557.

FIG. 558 shows a constriction device 68 having two rigid articulated clamping elements 69 positioned on opposite sides of a portion of a tubular uterine tube wall 70 of a patient's organ. An operation device 71 turns the clamping elements 69 toward each other to clamp the wall portion 70 between the clamping elements 69 to thereby contract the wall portion, and turns the clamping elements 69 away from each other to release the wall portion from the clamping elements 69. FIGS. 560 and 561 show an embodiment of the apparatus comprising a constriction device 300 having three bending members 301, 302 and 303 displaced relative to one another in a row along a portion of a tubular uterine tube wall 304 of a patient's uterine tubes and positioned alternately on opposite sides of the tubular wall 304. (Alternatively, each member 301, 302 and 303 may take the shape of an hour-glass.) An operation device (not shown) moves the two outer members 301, 303 laterally against the tubular wall 304 in one direction and the intermediate member 302 against the tubular wall 304 in the opposite direction to bend the tubular wall 304, to thereby constrict the tubular wall portion 304, as illustrated in FIG. 561. To release the wall portion 304 the operation device moves the members 301-303 away from the tubular wall portion 304 to the position shown in FIG. 560. FIGS. 562A and 562B show a hydraulically operable elongated constriction device in the form of a band 72 having an expandable/contractible cavity 73, which is in fluid communication with an adjustable reservoir 74 containing hydraulic fluid. FIG. 562A illustrates when the band is in a non-constriction state, whereas FIG. 562B illustrates when the band is in a constriction state, in which the cavity 73 is expanded by hydraulic fluid supplied by the reservoir 74. FIGS. 563A, 563B, 563C and 563D are block diagrams of four differently operated hydraulic constriction devices. FIG. 563A shows the band 72 of FIG. 563A, the cavity 73 of which is in fluid communication with a reservoir 75. FIG. 563B shows the embodiment of FIG. 563A, in which the cavity 73 of the band 72 is in fluid communication with the reservoir 74 via an operation device in the form of a two-way pump 76. FIG. 563C shows an operation device in the form of a reverse servo system with a first closed system controlling a second system. The reverse servo system comprises an adjustable fluid supply reservoir 77 and an adjustable servo reservoir 78. The servo reservoir 78 controls a larger adjustable reservoir 79 which in connection with the band 72 applied around a portion of tubular uterine tube wall of a patient's uterine tubes varies the volume of the cavity 73 of the band 72, which in turn varies the constriction of the wall portion. FIG. 563D shows an embodiment identical to the embodiment of FIG. 563C, except that the larger reservoir 79 is omitted. Instead, the servo reservoir 78 is in fluid communication with the cavity of the band 72. In all of the above embodiments according to FIGS. 544A through 30B, 562 stimulation devices may be provided to form constriction/stimulation units, in which the stimulation devices include a multiplicity of electrical elements 7 (indicated in FIGS. 544A-546, 549, 551-554, 557-563B) positioned on the constriction devices. FIG. 564 is a cross-sectional view of a fluid supply device including a bellows reservoir 80 defining a chamber 81, the size of which is variable by an operation device comprising a remote controlled electric motor 82. The reservoir 80 and the motor 82 are placed in a housing 83. Moving a large wall 84 varies the chamber 81. The wall 84 is secured to a nut 85, which is threaded on a rotatable spindle 86. The spindle 86 is rotated by the motor 82. A battery 89 placed in the housing 83 powers the motor 82. A signal receiver 90 for controlling the motor 82 is also placed in the housing 83. Alternatively, the battery 89 and the signal receiver 90 may be mounted in a separate place. The motor 82 may also be powered with energy transferred from transmitted signals. Where applicable, the fluid supply device of FIG. 564 may be used for supplying hydraulic fluid for the operation of the constriction devices described in this specification. For example, the fluid supply device of FIG. 564 may be substituted for the reservoir 74 in the embodiment according to FIG. 562A. FIGS. 565A and 565B show a reverse servo including a rectangular housing 91 and an intermediate wall 92, which is movable in the housing 91. A relatively large, substantially cylindrical bellows reservoir 93 is arranged in the housing 91 and is joined to the movable intermediate wall 92. Another cylindrical bellows reservoir 94, which is substantially smaller than reservoir 93, is arranged in the housing 91 at the other side of the intermediate wall 92 and is also joined to the wall 92. The small bellows reservoir 94 has a fluid supply pipe 95 and the large bellows reservoir 93 has a fluid supply pipe 96. Referring to FIG. 565A, when a small amount of hydraulic fluid is conducted through the supply pipe 95 into the small bellows reservoir 94, the small bellows reservoir 94 expands and pushes the movable intermediate wall 92 towards the large bellows reservoir 93. As a result, the large bellows reservoir 93 is contracted by the intermediate wall 92, whereby a large amount of hydraulic fluid is forced out of the large bellows reservoir 93 through the supply pipe 96, as shown in FIG. 565B. For example, the reverse servo of FIGS. 565A and 565B may be used in the embodiment of FIG. 563C, wherein the small bellows reservoir 94 corresponds to the small servo reservoir 78 and the large bellows reservoir 93 corresponds to the large reservoir 79. Also, the reverse servo of FIGS. 565A and 565B may be used in the embodiment of FIGS. 562A and 562B, wherein the small bellows reservoir 94 is connected to the adjustable reservoir 74, and the large bellows reservoir 93 is connected to the cavity 73 of the band 72. FIG. 566 schematically shows a hydraulically operable constriction device 97 of the apparatus of the invention, which is similar to the embodiment shown in FIG. 562, except that the hydraulic system is designed differently. Thus, the constriction device 97 includes a relatively small inflatable cavity 98, which is in fluid communication with a reservoir 99 containing hydraulic fluid, and a relatively large cavity 100, which is displaceable by small cavity 98. Small cavity 98 is adapted to displace large cavity 100 to constrict the patient's tubular wall portion when small cavity 98 is inflated and to displace large cavity 100 to release the wall portion when small cavity 98 is deflated. Thus, a relatively small addition of hydraulic fluid from reservoir 99 to small cavity 98 causes a relatively large increase in the constriction of the wall portion. Large cavity 100 is defined by a contraction element in the form of a big balloon 101, which may be connected to an injection port (not shown) for calibration of the volume of large cavity 100. Adding fluid to or withdrawing fluid from the injection port with the aid of a syringe calibrates the volume of balloon 101. Small cavity 98 is defined by a small bellows 102 attached to an annular frame 103 of constriction device 97 and at the opposite end is attached to balloon 101. FIGS. 567A and 567B schematically illustrate the operation of constriction device 97, when annular frame 103 is applied around the tubular wall portion of the patient's organ. Referring to FIG. 567A, when small cavity 98 is deflated bellows 102 pulls balloon 101 inwardly into annular frame 103, so that constriction device 97 constricts the wall portion. Referring to FIG. 567B, when small cavity 98 is inflated bellows 102 pulls balloon 101 out of annular frame 103, so that constriction device 97 releases the wall portion. As mentioned above, the constriction device and stimulation device can co-operate to actively move the sperms in the uterine tubes of a patient. This can be achieved using the constriction/stimulation unit shown in FIG. 534. Thus, in accordance with a first cooperation option, the clamping elements 5, 6 of the constriction device constricts the wall portion 8 without completely closing the uterine tube, whereby the flow in the uterine tube is restricted, and the control device 4 controls the electrical elements 7 to progressively stimulate the constricted wall portion in the downstream or upstream direction of the uterine tube to cause progressive contraction of the wall portion 8 to move the sperms in the uterine tube. In accordance with a second cooperation option, the constriction device constricts the wall portion so that the flow in the uterine tube is restricted, and the control device 4 controls a few electrical elements 7 at one end of the elongate clamping elements 5, 6 to stimulate the constricted wall portion 8 to close the uterine tube either at an upstream end or a downstream end of the wall portion 8. With the uterine tube closed in this manner, the control device 4 controls the constriction device to increase the constriction of the wall portion, whereby the sperms in the uterine tube are moved downstream or upstream of the wall portion 8. In another embodiment for performing the second cooperation option, the constriction device constricts the wall portion so that the flow in the uterine tube is restricted, and the control device 4 controls the stimulation device to stimulate the constricted wall portion while the constriction device vanes the constriction of the different areas of the wall portion, such that the wall portion is progressively constricted in the downstream or upstream direction of the uterine tube. FIGS. 568A—568E show different operation stages of such an alternative embodiment, which comprises a constriction device 104 including two elongate constriction elements 105, 106 having convex surfaces 107, 108 that abut a length of the wall portion 8 on mutual sides thereof, and a multiplicity of electrical elements 7 (such as electrodes) that are positioned on the convex surfaces 107, 108. The control device 4 controls the electrical elements 7 during operation of the constriction device 104 and controls the elongate constriction elements 105, 106 to move relative to the tubular wall portion 8 so that the constriction elements 105, 106 progressively constrict the wall portion 8, as appears from FIGS. 568A to 568D. Thus, in an initial position of the constriction elements 105, 106 shown in FIG. 568A, the wall portion is not constricted by the constriction elements 105, 106 and the electrical elements 7 are not energized. Starting from this initial position, the control device 4 controls the constriction elements 105, 106 to swing the left ends of the constriction elements 105, 106 toward the wall portion (indicated by arrows) to constrict the tubular wall portion 8, see FIG. 568B, while energizing the electrical elements 7, so that the electrical elements 7 that contact the wall portion 8 contract the latter. FIG. 568C shows how the uterine tube is completely closed by the thickened wall portion 8. Then, as shown in FIG. 568C, the control device 4 controls the constriction elements 105, 106 to move so that their right ends are moving towards each other (indicated by arrows), while the convex surfaces 107, 108 of the constriction elements 105, 106 are rolling on each other with the contracted wall portion 8 between them, see FIG. 568D. As a result, the sperm in the uterine tube of the organ is forced to the right (indicated by a white arrow). When the constriction elements 105, 106 have rolled on each other to the position shown in FIG. 568E, the control device 4 controls the right ends of the constriction elements 105, 106 to move away from each other (indicated by arrows in FIG. 568E) to the initial position shown in FIG. 568A. The operation stages described according to FIGS. 568A to 568E can be cyclically repeated a number of times until the desired amount of sperms has been moved in the uterine tube of the organ in a peristaltic manner. Alternatively, only one of the constriction elements 105, 106 can be provided with a convex surface, whereas the other constriction element has a plane surface that abuts the wall portion. It is also possible to use a single constriction element with a convex surface that presses the tubular portion 8 of the organ against a bone of the patient. In the embodiment according to FIGS. 568A to 568E, the control device 4 may control the electrical elements 7 to progressively stimulate the constricted wall portion 8 to cause progressive contraction thereof in harmony with the movement of the elongate constriction elements 105, 106, as the convex surfaces 107, 108 of the constriction elements 105, 106 are rolling on each other. FIG. 569 schematically shows a general embodiment of the apparatus of the invention, in which energy is transferred to energy consuming components of the apparatus implanted in the patient. The apparatus of FIG. 569 comprises an implanted constriction/stimulation unit 110, which is operable to gently constrict a portion of a tubular uterine tube wall of a patient and to stimulate different areas of the constricted portion to cause contraction of the wall portion. The constriction device of the constriction/stimulation unit 110 is capable of performing a reversible function, i.e., to constrict and release the wall portion. A source of energy 111 is adapted to supply energy consuming components of the constriction/stimulation unit 110 with energy via a power supply line 112. A wireless remote control or a subcutaneously implanted switch operable by the patient to switch on or off the supply of energy from the source of energy may be provided. The source of energy may be an implantable permanent or rechargeable battery, or be included in an external energy-transmission device, which may be operable directly by the patient or be controlled by a remote control operable by the patient to transmit wireless energy to the energy consuming components of the constriction/stimulation unit. Alternatively, the source of energy may comprise a combination of an implantable rechargeable battery, an external energy-transmission device and an implantable energy-transforming device for transforming wireless energy transmitted by the external energy-transmission device into electric energy for the charge of the implantable rechargeable battery. FIG. 570 shows a special embodiment of the general embodiment of FIG. 569 having some parts implanted in a patient and other parts located outside the patient's body. Thus, in FIG. 570 all parts placed to the right of the patient's skin 109 are implanted and all parts placed to the left of the skin 109 are located outside the patient's body. An implanted energy-transforming device 111A of the apparatus is adapted to supply energy consuming components of the constriction/stimulation unit 110 with energy via the power supply line 112. An external energy-transmission device 113 of the apparatus includes a wireless remote control transmitting a wireless signal, which is received by a signal receiver incorporated in the implanted energy-transforming device 111A. The implanted energy-transforming device 111A transforms energy from the signal into electric energy, which is supplied via the power supply line 112 to the constriction/stimulation unit 110. The apparatus of FIG. 570 may also include an implanted rechargeable battery for energizing energy consuming implanted components of the apparatus. In this case, the implanted energy-transforming device 111A also charges the battery with electric energy, as the energy-transforming device transforms energy from the signal into the electric energy. A reversing device in the form of an electric switch 114, such as a microprocessor, is implanted in the patient for reversing the constriction device of the constriction/stimulation unit 110. The wireless remote control of the external energy-transmission device 113 transmits a wireless signal that carries energy and the implanted energy-transforming device 111A transforms the wireless energy into a current for operating the switch 114. When the polarity of the current is shifted by the energy-transforming-device 111A the switch 114 reverses the function performed by the constriction device of the constriction/stimulation unit 110. FIG. 571 shows an embodiment including the energy-transforming device 111A, the constriction/stimulation unit 110 and an implanted operation device in the form of a motor 115 for operating the constriction device of the constriction/stimulation unit 110. The motor 115 is powered with energy from the energy-transforming device 111A, as the remote control of the external energy-transmission device 113 transmits a wireless signal to the receiver of the energy-transforming device 111A. FIG. 572 shows an embodiment including the energy-transforming device 111A, the constriction/stimulation unit 110 and an implanted assembly 116 including a motor/pump unit 117 and a fluid reservoir 118. In this case the constriction device of the constriction/stimulation unit 110 is hydraulically operated, i.e., hydraulic fluid is pumped by the motor/pump unit 117 from the reservoir 118 to the constriction/stimulation unit 110 to constrict the wall portion, and hydraulic fluid is pumped by the motor/pump unit 117 back from the constriction/stimulation unit 110 to the reservoir 118 to release the wall portion. The implanted energy-transforming device 111A transforms wireless energy into a current, for powering the motor/pump unit 117. FIG. 573 shows an embodiment comprising the external energy-transmission device 113 that controls the control unit 122 to reverse the motor 115 when needed, the constriction/stimulation unit 110, the constriction device of which is hydraulically operated, and the implanted energy-transforming device 111A, and further comprising an implanted hydraulic fluid reservoir 119, an implanted motor/pump unit 120, an implanted reversing device in the form of a hydraulic valve shifting device 121 and a separate external wireless remote control 111B. The motor of the motor/pump unit 120 is an electric motor. In response to a control signal from the wireless remote control of the external energy-transmission device 113, the implanted energy-transforming device 111A powers the motor/pump unit 120 with energy from the energy carried by the control signal, whereby the motor/pump unit 120 distributes hydraulic fluid between the reservoir 119 and the constriction device of the constriction/stimulation unit 110. The remote control 111B controls the shifting device 121 to shift the hydraulic fluid flow direction between one direction in which the fluid is pumped by the motor/pump unit 120 from the reservoir 119 to the constriction device of the constriction/stimulation unit 110 to constrict the wall portion, and another opposite direction in which the fluid is pumped by the motor/pump unit 120 back from the constriction device of the constriction/stimulation unit 110 to the reservoir 119 to release the wall portion. FIG. 574 shows an embodiment including the energy-transforming device 111A and the constriction/stimulation unit 110. A control unit 122, an accumulator 123 and a capacitor 124 are also implanted in the patient. A separate external wireless remote control 111B controls the control unit 122. The control unit 122 controls the energy-transforming device 111A to store electric energy in the accumulator 123, which supplies energy to the constriction/stimulation unit 110. In response to a control signal from the wireless remote control 111B, the control unit 122 either releases electric energy from the accumulator 123 and transfers the released energy via power lines, or directly transfers electric energy from the energy-transforming device 111A via the capacitor 124, which stabilizes the electric current, for the operation of the constriction/stimulation unit 110. In accordance with one alternative, the capacitor 124 in the embodiment of FIG. 574 may be omitted. In accordance with another alternative, the accumulator 123 in this embodiment may be omitted. FIG. 575 shows an embodiment including the energy-transforming device 111A, the constriction/stimulation unit 110. A battery 125 for supplying energy for the operation of the constriction/stimulation unit 110 and an electric switch 126 for switching the operation of the constriction/stimulation unit 110 are also implanted in the patient. The switch 126 is operated by the energy supplied by the energy-transforming device 111A to switch from an off mode, in which the battery 125 is not in use, to an on mode, in which the battery 125 supplies energy for the operation of the constriction/stimulation unit 110. FIG. 576 shows an embodiment identical to that of FIG. 575, except that a control unit 122 also is implanted in the patient. A separate external wireless remote control 111B controls the control unit 122. In this case, the switch 126 is operated by the energy supplied by the energy-transforming device 111A to switch from an off mode, in which the wireless remote control 111B is prevented from controlling the control unit 122 and the battery 125 is not in use, to a standby mode, in which the remote control 111B is permitted to control the control unit 122 to release electric energy from the battery 125 for the operation of the constriction/stimulation unit 110. FIG. 577 shows an embodiment identical to that of FIG. 576, except that the accumulator 123 is substituted for the battery 125 and the implanted components are interconnected differently. In this case, the accumulator 123 stores energy from the energy-transforming device 111A. In response to a control signal from the wireless remote control 111B, the implanted control unit 122 controls the switch 126 to switch from an off mode, in which the accumulator 123 is not in use, to an on mode, in which the accumulator 123 supplies energy for the operation of the constriction/stimulation unit 110. FIG. 578 shows an embodiment identical to that of FIG. 577, except that the battery 125 also is implanted in the patient, and the implanted components are interconnected differently. In response to a control signal from the wireless remote control 111B, the implanted control unit 122 controls the accumulator 123, which may be a capacitor, to deliver energy for operating the switch 126 to switch from an off mode, in which the battery 125 is not in use, to an on mode, in which the battery 125 supplies electric energy for the operation of the constriction/stimulation unit 110. Alternatively, the switch 126 may be operated by energy supplied by the accumulator 123 to switch from an off mode, in which the wireless remote control 111B is prevented from controlling the battery 125 to supply electric energy and the battery 125 is not in use, to a standby mode, in which the wireless remote control 111B is permitted to control the battery 125 to supply electric energy for the operation of the constriction/stimulation unit 110. FIG. 579 shows an embodiment identical to that of FIG. 575, except that a motor 115, a mechanical reversing device in the form of a gearbox 127 and a control unit 122 for controlling the gearbox 127 also are implanted in the patient. A separate external wireless remote control 111B controls the implanted control unit 122 to control the gearbox 127 to reverse the function performed by the constriction device (mechanically operated) of the constriction/stimulation unit 110. FIG. 580 shows an embodiment identical to that of FIG. 578, except that the implanted components are interconnected differently. Thus, in this case, the battery 125 powers the control unit 122 when the accumulator 123, suitably a capacitor, activates the switch 126 to switch to an on mode. When the switch 126 is in its on mode the control unit 122 is permitted to control the battery 125 to supply, or not supply, energy for the operation of the constriction/stimulation unit 110. FIG. 581 shows an embodiment identical to that of FIG. 579, except that a gearbox 127 that connects the motor 115 to the constriction/stimulation unit 110, and a control unit 122 that controls the energy-transforming device 111A to power the motor 115 also are implanted in the patient. There is a separate external wireless remote control 111B that controls the control unit 122 to reverse the motor 115 when needed.

Optionally, the accumulator 123 shown in FIG. 574 may be provided in the embodiment of FIG. 581, wherein the implanted control unit 122 controls the energy-transforming device 111A to store the transformed energy in the accumulator 123. In response to a control signal from the wireless remote control 111B, the control unit 122 controls the accumulator 123 to supply energy for the operation of the constriction/stimulation unit 110. Those skilled in the art will realize that the above various embodiments according to FIGS. 570-581 could be combined in many different ways. For example, the energy operated switch 114 could be incorporated in any of the embodiments of FIGS. 571, 574-581, the hydraulic shifting device 121 could be incorporated in the embodiment of FIG. 572, and the gearbox 127 could be incorporated in the embodiment of FIG. 571. The switch 114 may be of a type that includes electronic components, for example a microprocessor, or a FGPA (Field Programmable Gate Array) designed for switching. Alternatively, however, the energy operated switch 114 may be replaced by a subcutaneously implanted push button that is manually switched by the patient between “on” and “off”. Alternatively, a permanent or rechargeable battery may be substituted for the energy-transforming devices 111A of the embodiments shown in FIGS. 570-581. FIG. 582 shows the energy-transforming device in the form of an electrical junction element 128 for use in any of the above embodiments according to FIGS. 569-581. The element 128 is a flat p-n junction element comprising a p-type semiconductor layer 129 and an n-type semiconductor layer 130 sandwiched together. A light bulb 131 is electrically connected to opposite sides of the element 128 to illustrate how the generated current is obtained. The output of current from such a p-n junction element 128 is correlated to the temperature. See the formula below.

$$I = I 0 ( exp ( qV / kT ) - 1 )$$

Where
I is the external current flow,
is the reverse saturation current,
q is the fundamental electronic charge of 1.602×10-19 coulombs,
V is the applied voltage,
k is the Boltzmann constant, and
T is the absolute temperature.

Under large negative applied voltage (reverse bias), the exponential term becomes negligible compared to 1.0, and I is approximately −I0. I0 is strongly dependent on the temperature of the junction and hence on the intrinsic-carrier concentration. I0 is larger for materials with smaller bandgaps than for those with larger bandgaps. The rectifier action of the diode, that is, its restriction of current flow to only one direction, is in this particular embodiment the key to the operation of the p-n junction element 128.

The alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers, such as, but not limited to, cadmium telluride, copper-indium-diselenide and silicon. It is also possible to use a multilayer structure with several layers of p and n-type materials to improve efficiency.

The electric energy generated by the p-n junction element 128 could be of the same type as generated by solar cells, in which the negative and positive fields create a direct current. Alternatively, the negative and positive semiconductor layers may change polarity following the transmitted waves, thereby generating the alternating current.

The p-n junction element 128 is designed to make it suited for implantation. Thus, all the external surfaces of the element 128 in contact with the human body are made of a biocompatible material. The p-n junction semiconductors are designed to operate optimally at a body temperature of 37° C. because the current output, which should be more than 1 μA, is significantly dependent upon such temperature, as shown above. Since both the skin and subcutis absorb energy, the relation between the sensitivity or working area of the element 128 and the intensity or strength of the wireless energy-transmission is considered. The p-n junction element 128 preferably is designed flat and small. Alternatively, if the element 128 is made in larger sizes it should be flexible, in order to adapt to the patient's body movements. The volume of the element 128 should be kept less than 2000 cm³. FIG. 583 shows basic parts of a remote control of the apparatus for controlling the constriction/stimulation unit 110. In this case, the stimulation device of the constriction/stimulation unit stimulates the wall portion with electric pulses. The remote control is based on wireless transmission of electromagnetic wave signals, often of high frequencies in the order of 100 kHz-1 gHz, through the skin 132 of the patient. In FIG. 583, all parts placed to the left of the skin 132 are located outside the patient's body and all parts placed to the right of the skin 132 are implanted. An external signal-transmission device 133 is to be positioned close to a signal-receiving device 134 implanted close to the skin 132. As an alternative, the signal-receiving device 134 may be placed for example inside the abdomen of the patient. The signal-receiving device 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The signal transmission device 133 comprises a coil having about the same size as the coil of the signal-receiving device 134 but wound with a thick wire that can handle the larger currents that is necessary. The coil of the signal transmission device 133 is tuned to the same specific high frequency as the coil of the signal-receiving device 134.

The signal-transmission device 133 is adapted to send digital information via the power amplifier and signal-receiving device 134 to an implanted control unit 135. To avoid that accidental random high frequency fields trigger control commands, digital signal codes are used. A conventional keypad placed on the signal transmission device 133 is used to order the signal transmission device 133 to send digital signals for the control of the constriction/stimulation unit. The signal transmission device 133 starts a command by generating a high frequency signal. After a short time, when the signal has energized the implanted parts of the control system, commands are sent to operate the constriction device of the constriction/stimulation unit 110 in predefined steps. The commands are sent as digital packets in the form illustrated below.

| | | | |
|---|---|---|---|
| Start | Command, | Count, | Check sum, |
| pattern, | 8 bits | 8 bits | 8 bits |
| 8 bits | | | |

The commands are sent continuously during a rather long time period (e.g., about 30 seconds or more). When a new constriction or release step is desired, the Count byte is increased by one to allow the implanted control unit 135 to decode and understand that another step is demanded by the signal transmission device 133. If any part of the digital packet is erroneous, its content is simply ignored.

Through a line 136, an implanted energizer unit 137 draws energy from the high frequency electromagnetic wave signals received by the signal-receiving device 134. The energizer unit 137 stores the energy in a source of energy, such as a large capacitor, powers the control unit 135 and powers the constriction/stimulation unit 110 via a line 138.

The control unit 135 comprises a demodulator and a microprocessor. The demodulator demodulates digital signals sent from the signal transmission device 133. The microprocessor receives the digital packet, decodes it and sends a control signal via a signal line 139 to control the constriction device of the constriction/stimulation unit 110 to either constrict or release the wall portion of the patient's organ depending on the received command code.

FIG. 584 shows a circuitry of an embodiment of the invention, in which wireless energy is transformed into a current. External components of the circuitry include a microprocessor 140, a signal generator 141 and a power amplifier 142 connected thereto. The microprocessor 140 is adapted to switch the signal generator 141 on/off and to modulate signals generated by the signal generator 141 with digital commands. The power amplifier 142 amplifies the signals and sends them to an external signal-transmitting antenna coil 143. The antenna coil 143 is connected in parallel with a capacitor 144 to form a resonant circuit tuned to the frequency generated by the signal generator 141.

Implanted components of the circuitry include a signal receiving antenna coil 145 and a capacitor 146 forming together a resonant circuit that is tuned to the same frequency as the transmitting antenna coil 143. The signal receiving antenna coil 145 induces a current from the received high frequency electromagnetic waves and a rectifying diode 147 rectifies the induced current, which charges a storage capacitor 148. The storage capacitor 148 powers a motor 149 for driving the constriction device of the constriction/stimulation unit 110. A coil 150 connected between the antenna coil 145 and the diode 147 prevents the capacitor 148 and the diode 147 from loading the circuit of the signal-receiving antenna 145 at higher frequencies. Thus, the coil 150 makes it possible to charge the capacitor 148 and to transmit digital information using amplitude modulation.

A capacitor 151 and a resistor 152 connected in parallel and a diode 153 form a detector used to detect amplitude modulated digital information. A filter circuit is formed by a resistor 154 connected in series with a resistor 155 connected in series with a capacitor 156 connected in series with the resistor 154 via ground, and a capacitor 157, one terminal of which is connected between the resistors 154, 155 and the other terminal of which is connected between the diode 153 and the circuit formed by the capacitor 151 and resistor 152. The filter circuit is used to filter out undesired low and high frequencies. The detected and filtered signals are fed to an implanted microprocessor 158 that decodes the digital information and controls the motor 149 via an H-bridge 159 comprising transistors 160, 161, 162 and 163. The motor 149 can be driven in two opposite directions by the H-bridge 159. The microprocessor 158 also monitors the amount of stored energy in the storage capacitor 148. Before sending signals to activate the motor 149, the microprocessor 158 checks whether the energy stored in the storage capacitor 148 is enough. If the stored energy is not enough to perform the requested operation, the microprocessor 158 waits for the received signals to charge the storage capacitor 148 before activating the motor 149. Alternatively, the energy stored in the storage capacitor 148 may only be used for powering a switch, and the energy for powering the motor 149 may be obtained from another implanted energy source of relatively high capacity, for example a battery. In this case the switch is adapted to connect the battery to the motor 149 in an on mode when the switch is powered by the storage capacitor 148 and to keep the battery disconnected from the motor 149 in a standby mode when the switch is not powered. FIGS. 585A-585C show an embodiment of the invention, which is similar to the embodiment of FIG. 534, except that the constriction/stimulation unit, here denoted by reference numeral 200, is provided with additional clamping elements. The embodiment of FIGS. 585A-585C is suited for actively moving the sperms in the uterine tube of a patient. Thus, the constriction/stimulation unit 200 also includes a first pair of short clamping elements 201 and 202, and a second pair of short clamping elements 203 and 204, wherein the first and second pairs of clamping elements are positioned at mutual sides of the elongate clamping elements 5, 6. The two short clamping elements 201, 202 of the first pair are radially movable towards and away from each other between retracted positions (FIG. 585A) and clamping positions (FIGS. 585B and 585C), and the two short clamping elements 203, 204 of the second pair are radially movable towards and away from each other between retracted positions (FIG. 585C) and clamping positions (FIGS. 585A and 585B). The stimulation device 3 also includes electrical elements 7 positioned on the short clamping elements 201-204, so that the electrical elements 7 on one of the short clamping elements 201 and 203, respectively, of each pair of short elements face the electrical elements 7 on the other short clamping element 202 and 204, respectively, of each pair of short elements. The constriction/stimulation unit 200 is applied on a wall portion 8 of a tubular uterine tube wall of a patient, so that the short clamping elements 201, 202 are positioned at an upstream end of the wall portion 8, whereas the short clamping elements 203, 204 202 are positioned at a downstream end of the wall portion 8. In FIGS. 585A to 585C the upstream end of the wall portion 8 is to the left and the downstream end of the wall portion 8 is to the right. The control device 4 controls the pair of short clamping elements 201, 202, the pair of elongate clamping elements 5, 6 and the pair of short elements 203, 204 to constrict and release the wall portion 8 independently of one another. The control device also controls the electrical elements 7 on a clamping element that is constricting the wall portion to stimulate the constricted wall portion 8 with electric pulses to cause contraction of the wall portion 8, so that the uterine tube of the wall portion 8 is closed. FIGS. 585A-585C illustrate how the control device 4 controls the operation of the constriction/stimulation unit 200 to cyclically move sperms downstream in the uterine tube of the wall portion 8. Thus, in FIG. 585A the short clamping elements 201, 202 and the elongate clamping elements 5, 6 are in their retracted positions, whereas the short clamping elements 203, 204 are in their clamping positions while the electrical elements 7 on elements 203, 204 electrically stimulate the wall portion 8. The electrical stimulation causes the wall portion 8 at the elements 203, 204 to thicken, whereby the uterine tube is closed. FIG. 585B illustrates how also the short clamping elements 201, 202 have been moved radially inwardly to their clamping positions, while the electrical elements 7 on elements 201, 202 electrically stimulate the wall portion 8, whereby a volume of bodily matter is trapped in the uterine tube between the upstream and downstream ends of the wall portion 8. FIG. 585C illustrates how initially the short clamping elements 203, 204 have been moved radially outwardly to their retracted positions, and then the elongate clamping elements 5, 6 have been moved radially inwardly to their clamping positions while the electrical elements 7 on elements 5, 6 electrically stimulate the wall portion 8. As a result, the sperms in the uterine tube between the upstream and downstream ends of the wall portion 8 have been moved downstream in the uterine tube Then, the control device 4 controls the constriction/stimulation unit 200 to assume the state shown in FIG. 585A. Alternatively, the operation cycle of the constriction/stimulation unit 200 described above may be reversed, in order to move a sperm upstream in the uterine tube. In this case, the control device 4 controls the short clamping elements 203, 204 to constrict the wall portion 8 at the downstream end thereof to restrict the flow in the uterine tube and controls the electric elements 7 to stimulate the constricted wall portion 8 with electric pulses at the downstream end to close the uterine tube. With the uterine tube closed at the downstream end of the constricted wall portion 8 and the short clamping elements 201, 202 in their retracted positions, as shown in FIG. 585A, the control device 4 controls the elongate clamping elements 5, 6 to constrict the wall portion 8 between the upstream and downstream ends thereof. As a result, the sperm contained in the wall portion 8 between the upstream and downstream ends thereof is moved upstream in the uterine tube. Although FIGS. 585A-585C disclose pairs of clamping elements, it should be noted that it is conceivable to design the constriction/stimulation unit 200 with only a single short clamping element 201, a single elongate clamping element 5 and a single short clamping element 203. In this case the bottom of the tubular wall portion 8 is supported by stationary elements of the constriction/stimulation unit 200 opposite to the clamping elements 201, 5, and 203. FIGS. 585A and 585B schematically show another embodiment of the invention, in which a constriction/stimulation unit 205 is designed for actively moving sperms in the uterine tube of a patient's tubular organ. The constriction device 206 of the constriction/stimulation unit 205 includes a rotor 207, which carries three cylindrical constriction elements 208A, 208B and 208C positioned equidistantly from the axis 209 of the rotor 207. The constriction elements 208A-208C may be designed as rollers. Each cylindrical element 208A-208C is provided with electrical elements 7. A stationary elongate support element 210 is positioned spaced from but close to the rotor 207 and has a part cylindrical surface 211 concentric with the axis 209 of the rotor 207. The constriction/stimulation unit 205 is applied on a patient's uterine tube 212, so that the organ 212 extends between the support element 210 and the rotor 207. The control device 4 controls the rotor 207 of the constriction device to rotate, such that the constriction elements 208A-208C successively constrict wall portions of a series of wall portions of the uterine tube 212 against the elongate support element 210. The electrical elements 7 of the constriction elements 208A-208C stimulate the constricted wall portions with electric pulses so that the wall portions thicken and close the uterine tube 212. FIG. 586A illustrates how the constriction element 208A has started to constrict the wall of the uterine tube 212 and how the uterine tube 212 is closed with the aid of the electrical elements 7 on the constriction element 208A, whereas the constriction element 208B is about to release the uterine tube 212. FIG. 586B illustrates how the constriction element 208A has advanced about halfway along the elongate support element 210 and moved the sperm in the uterine tube in a direction indicated by an arrow. The constriction element 208B has released the uterine tube 212, whereas the constriction element 208C is about to engage the uterine tube 212. Thus, the control device 4 controls the rotor 207 to cyclically move the constriction elements 208A-208C, one after the other, along the elongate support element 210, while constricting the wall portions of the uterine tube 212, so that the sperm in the 212 is moved in a peristaltic manner. FIGS. 587A, 587B and 587C show another mechanically operable constriction device 213 for use in the apparatus of the invention. Referring to FIG. 587A, the constriction device 213 includes a first ring-shaped holder 214 applied on a uterine tube 8 of a patient and a second ring-shaped holder 215 also applied on the uterine tube 8 spaced apart from holder 214. There are elastic strings 216 (here twelve strings) that extend in parallel along the uterine tube 8 and interconnect the two holders 213, 214 without contacting the uterine tube 8. FIG. 587A illustrate an inactivated state of the constriction device 213 in which the uterine tube 8 is not constricted. Referring to FIGS. 587B and 587C, when the uterine tube 8 is to be constricted the ring-shaped holders 214 and 215 are rotated by an operation means (not shown) in opposite directions, whereby the elastic strings 216 constrict the uterine tube 8 in a manner that appears from FIGS. 587B and 587C. For the sake of clarity, only five strings 216 are shown in FIG. 587B. In accordance with the present invention, electrodes for electrically stimulating the uterine tube 8 to cause contraction of the wall of the uterine tube 8 are attached to the strings 216 (not shown in FIGS. 587A-587C). FIG. 588 schematically illustrates an arrangement of the apparatus that is capable of sending information from inside the patient's body to the outside thereof to give information related to at least one functional parameter of the apparatus, and/or related to a physical parameter of the patient, in order to supply an accurate amount of energy to an implanted internal energy receiver 302 connected to energy consuming components of an implanted constriction/stimulation unit 301 of the apparatus of the invention. Such an energy receiver 302 may include a source of energy and/or an energy-transforming device. Briefly described, wireless energy is transmitted from an external source of energy 304a located outside the patient and is received by the internal energy receiver 302 located inside the patient. The internal energy receiver is adapted to directly or indirectly supply received energy to the energy consuming components of the constriction/stimulation unit 301 via a switch 326. An energy balance is determined between the energy received by the internal energy receiver 302 and the energy used for the constriction/stimulation unit 301, and the transmission of wireless energy is then controlled based on the determined energy balance. The energy balance thus provides an accurate indication of the correct amount of energy needed, which is sufficient to operate the constriction/stimulation unit 301 properly, but without causing undue temperature rise. In FIG. 588 the patient's skin is indicated by a vertical line 305. Here, the energy receiver comprises an energy-transforming device 302 located inside the patient, preferably just beneath the patient's skin 305. Generally speaking, the implanted energy-transforming device 302 may be placed in the abdomen, thorax, muscle fascia (e.g. in the abdominal wall), subcutaneously, or at any other suitable location. The implanted energy-transforming device 302 is adapted to receive wireless energy E transmitted from the external source of energy 304a provided in an external energy-transmission device 304 located outside the patient's skin 305 in the vicinity of the implanted energy-transforming device 302. As is well known in the art, the wireless energy E may generally be transferred by means of any suitable Transcutaneous Energy Transfer (TET) device, such as a device including a primary coil arranged in the external source of energy 304a and an adjacent secondary coil arranged in the implanted energy-transforming device 302. When an electric current is fed through the primary coil, energy in the form of a voltage is induced in the secondary coil which can be used to power the implanted energy consuming components of the apparatus, e.g. after storing the incoming energy in an implanted source of energy, such as a rechargeable battery or a capacitor. However, the present invention is generally not limited to any particular energy transfer technique, TET devices or energy sources, and any kind of wireless energy may be used. The amount of energy received by the implanted energy receiver may be compared with the energy used by the implanted components of the apparatus. The term “energy used” is then understood to include also energy stored by implanted components of the apparatus. A control device includes an external control unit 304b that controls the external source of energy 304a based on the determined energy balance to regulate the amount of transferred energy. In order to transfer the correct amount of energy, the energy balance and the required amount of energy is determined by means of a determination device including an implanted internal control unit 315 connected between the switch 326 and the constriction/stimulation unit 301. The internal control unit 315 may thus be arranged to receive various measurements obtained by suitable sensors or the like, not shown, measuring certain characteristics of the constriction/stimulation unit 301, somehow reflecting the required amount of energy needed for proper operation of the constriction/stimulation unit 301. Moreover, the current condition of the patient may also be detected by means of suitable measuring devices or sensors, in order to provide parameters reflecting the patient's condition. Hence, such characteristics and/or parameters may be related to the current state of the constriction/stimulation unit 301, such as power consumption, operational mode and temperature, as well as the patient's condition reflected by parameters such as: body temperature, blood pressure, heartbeats and breathing. Other kinds of physical parameters of the patient and functional parameters of the device are described elsewhere. Furthermore, a source of energy in the form of an accumulator 316 may optionally be connected to the implanted energy-transforming device 302 via the control unit 315 for accumulating received energy for later use by the constriction/stimulation unit 301. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the constriction/stimulation unit 301, and also to avoid excessive heating, it is clearly understood that the battery should be charged optimally by receiving a correct amount of energy from the implanted energy-transforming device 302, i.e. not too little or too much. The accumulator may also be a capacitor with corresponding characteristics. For example, battery characteristics may be measured on a regular basis to determine the current state of the battery, which then may be stored as state information in a suitable storage means in the internal control unit 315. Thus, whenever new measurements are made, the stored battery state information can be updated accordingly. In this way, the state of the battery can be “calibrated” by transferring a correct amount of energy, so as to maintain the battery in an optimal condition. Thus, the internal control unit 315 of the determination device is adapted to determine the energy balance and/or the currently required amount of energy, (either energy per time unit or accumulated energy) based on measurements made by the above-mentioned sensors or measuring devices of the apparatus, or the patient, or an implanted source of energy if used, or any combination thereof. The internal control unit 315 is further connected to an internal signal transmitter 327, arranged to transmit a control signal reflecting the determined required amount of energy, to an external signal receiver 304c connected to the external control unit 304b. The amount of energy transmitted from the external source of energy 304a may then be regulated in response to the received control signal. Alternatively, the determination device may include the external control unit 304b. In this alternative, sensor measurements can be transmitted directly to the external control unit 304b wherein the energy balance and/or the currently required amount of energy can be determined by the external control unit 304b, thus integrating the above-described function of the internal control unit 315 in the external control unit 304b. In that case, the internal control unit 315 can be omitted and the sensor measurements are supplied directly to the internal signal transmitter 327 which sends the measurements over to the external signal receiver 304c and the external control unit 304b. The energy balance and the currently required amount of energy can then be determined by the external control unit 304b based on those sensor measurements. Hence, the present solution according to the arrangement of FIG. 588 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted source of energy or the like. The different parameters discussed above would thus be used if relevant and needed and then as a tool for determining the actual energy balance. However, such parameters may also be needed per se for any actions taken internally to specifically operate the apparatus. The internal signal transmitter 327 and the external signal receiver 304c may be implemented as separate units using suitable signal transfer means, such as radio, IR (Infrared) or ultrasonic signals. Alternatively, the internal signal transmitter 327 and the external signal receiver 304c may be integrated in the implanted energy-transforming device 302 and the external source of energy 304a, respectively, so as to convey control signals in a reverse direction relative to the energy transfer, basically using the same transmission technique. The control signals may be modulated with respect to frequency, phase or amplitude.

Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off. In implementing this system in the arrangement of FIG. 548, the switch 326 is either separate and controlled by the internal control unit 315, or integrated in the internal control unit 315. It should be understood that the switch 326 should be interpreted in its broadest embodiment. This means a transistor, MCU, MCPU, ASIC FPGA or a DA converter or any other electronic component or circuit that may switch the power on and off. To conclude, the energy supply arrangement illustrated in FIG. 588 may operate basically in the following manner. The energy balance is first determined by the internal control unit 315 of the determination device. A control signal reflecting the required amount of energy is also created by the internal control unit 315, and the control signal is transmitted from the internal signal transmitter 327 to the external signal receiver 304c. Alternatively, the energy balance can be determined by the external control unit 304b instead depending on the implementation, as mentioned above. In that case, the control signal may carry measurement results from various sensors. The amount of energy emitted from the external source of energy 304a can then be regulated by the external control unit 304b, based on the determined energy balance, e.g. in response to the received control signal. This process may be repeated intermittently at certain intervals during ongoing energy transfer, or may be executed on a more or less continuous basis during the energy transfer. The amount of transferred energy can generally be regulated by adjusting various transmission parameters in the external source of energy 304a, such as voltage, current, amplitude, wave frequency and pulse characteristics. This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized. This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor. With reference to FIG. 589, although wireless transfer of energy for operating the apparatus has been described above to enable non-invasive operation, it will be appreciated that the apparatus can be operated with wire bound energy as well. Such an example is shown in FIG. 589, wherein an external switch 326 is interconnected between the external source of energy 304a and an operation device, such as an electric motor 307 operating the constriction/stimulation unit 301. An external control unit 304b controls the operation of the external switch 326 to effect proper operation of the constriction/stimulation unit 301. FIG. 589 illustrates different embodiments for how received energy can be supplied to and used by the constriction/stimulation unit 301. Similar to the example of FIG. 588, an internal energy receiver 302 receives wireless energy E from an external source of energy 304a which is controlled by a transmission control unit 304b. The internal energy receiver 302 may comprise a constant voltage circuit, indicated as a dashed box “constant V” in FIG. 590, for supplying energy at constant voltage to the constriction/stimulation unit 301. The internal energy receiver 302 may further comprise a constant current circuit, indicated as a dashed box “constant C” in the figure, for supplying energy at constant current to the constriction/stimulation unit 301. The constriction/stimulation unit 301 comprises an energy consuming part 301a, which may be a motor, pump, restriction device, or any other medical appliance that requires energy for its electrical operation. The constriction/stimulation unit 301 may further comprise an energy storage device 301b for storing energy supplied from the internal energy receiver 302. Thus, the supplied energy may be directly consumed by the energy consuming part 301a, or stored by the energy storage device 301b, or the supplied energy may be partly consumed and partly stored. The constriction/stimulation unit 301 may further comprise an energy stabilizing unit 301c for stabilizing the energy supplied from the internal energy receiver 302. Thus, the energy may be supplied in a fluctuating manner such that it may be necessary to stabilize the energy before consumed or stored. The energy supplied from the internal energy receiver 302 may further be accumulated and/or stabilized by a separate energy stabilizing unit 328 located outside the constriction/stimulation unit 301, before being consumed and/or stored by the constriction/stimulation unit 301. Alternatively, the energy stabilizing unit 328 may be integrated in the internal energy receiver 302. In either case, the energy stabilizing unit 328 may comprise a constant voltage circuit and/or a constant current circuit. It should be noted that FIG. 588 and FIG. 590 illustrate some possible but non-limiting implementation options regarding how the various shown functional components and elements can be arranged and connected to each other. However, the skilled person will readily appreciate that many variations and modifications can be made within the scope of the present invention. FIG. 591 schematically shows an energy balance measuring circuit of one of the proposed designs of the apparatus for controlling transmission of wireless energy, or energy balance. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the source of energy. The output signal from the circuit is typically fed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window. The schematic FIG. 591 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 591; the transmitting parts of the system are excluded. The implementation of the general concept of energy balance and the way the information is transmitted to the external energy transmitter can of course be implemented in numerous different ways. The schematic FIG. 551 and the above described method of evaluating and transmitting the information should only be regarded as examples of how to implement the control system.

### Circuit Details

In FIG. 591 the symbols Y1, Y2, Y3 and so on symbolize test points within the circuit. The components in the diagram and their respective values are values that work in this particular implementation which of course is only one of an infinite number of possible design solutions. Energy to power the circuit is received by the energy receiving coil L1. Energy to implanted components is transmitted in this particular case at a frequency of 25 kHz. The energy balance output signal is present at test point Y1. The embodiments described in connection with FIGS. 588, 590 and 591 identify a general method for controlling transmission of wireless energy to implanted energy consuming components of the apparatus of the present invention. Such a method will be defined in general terms in the following. A method is thus provided for controlling transmission of wireless energy supplied to implanted energy consuming components of an apparatus as described above. The wireless energy E is transmitted from an external source of energy located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the operation of the implanted parts of the apparatus. The transmission of wireless energy E from the external source of energy is then controlled based on the determined energy balance. The wireless energy may be transmitted inductively from a primary coil in the external source of energy to a secondary coil in the internal energy receiver. A change in the energy balance may be detected to control the transmission of wireless energy based on the detected energy balance change. A difference may also be detected between energy received by the internal energy receiver and energy used for the operation of the implanted parts of the apparatus, to control the transmission of wireless energy based on the detected energy difference. When controlling the energy transmission, the amount of transmitted wireless energy may be decreased if the detected energy balance change implies that the energy balance is increasing, or vice versa. The decrease/increase of energy transmission may further correspond to a detected change rate. The amount of transmitted wireless energy may further be decreased if the detected energy difference implies that the received energy is greater than the used energy, or vice versa. The decrease/increase of energy transmission may then correspond to the magnitude of the detected energy difference. As mentioned above, the energy used for the operation of the implanted parts of the apparatus be consumed to operate the implanted parts of the apparatus and/or stored in at least one implanted energy storage device of the apparatus. When electrical and/or physical parameters of the implanted parts of the apparatus and/or physical parameters of the patient are determined, the energy may be transmitted for consumption and storage according to a transmission rate per time unit which is determined based on the parameters. The total amount of transmitted energy may also be determined based on the parameters. When a difference is detected between the total amount of energy received by the internal energy receiver and the total amount of consumed and/or stored energy, and the detected difference is related to the integral over time of at least one measured electrical parameter related to the energy balance, the integral may be determined for a monitored voltage and/or current related to the energy balance. When the derivative is determined over time of a measured electrical parameter related to the amount of consumed and/or stored energy, the derivative may be determined for a monitored voltage and/or current related to the energy balance. The transmission of wireless energy from the external source of energy may be controlled by applying to the external source of energy electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals. In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses. A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency. The circuit formed by the first electric circuit and the external source of energy may have a first characteristic time period or first time constant, and when effectively varying the transmitted energy, such frequency time period may be in the range of the first characteristic time period or time constant or shorter. The embodiments described in connection with FIGS. 588, 590 and 591 also identify general features for controlling transmission of wireless energy to implanted energy consuming components of the apparatus of the present invention. Such features of the apparatus will be defined in general terms in the following. In its broadest sense, the apparatus comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The apparatus further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

Further, the apparatus may comprise any of the following features:
A primary coil in the external source of energy adapted to transmit the wireless energy inductively to a secondary coil in the internal energy receiver.
The determination device is adapted to detect a change in the energy balance, and the control device controls the transmission of wireless energy based on the detected energy balance change.
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of the detected energy difference.
The energy used for implanted parts of the apparatus is consumed to operate the implanted parts, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on the parameters. The determination device also determines the total amount of transmitted energy based on the parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, the train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

A constriction device can be arranged to delay the movement of the sperms towards the egg for a predetermined amount of time in order to avoid pregnancy. This can be achieved in many different ways, of which two will be described below. FIG. 592 is a sectional view through a constriction device 2 adapted to restrict or stop the flow through a uterine tube. The general flow direction is illustrated by an arrow. The constriction device comprises an array of constriction elements 2a-2n, each arranged to restrict or close a part of the uterine tube. The constriction device illustrated in FIG. 592 is in an open or non-operative position wherein the flow is uninterrupted FIG. 593A illustrates the constriction device of FIG. 592 in a first interrupting stage, wherein every other constriction element is in a closed position. A sperm, generally designated 1000, is allowed to enter the space formed by the first, non-closed constriction element. It is stopped there by the second constriction element, which is in a closed position. This operative state can remain for a desired period of time, such as one day. FIG. 593B illustrates the constriction device of FIG. 592 in a second interrupting stage, wherein every constriction element that was closed in the first interrupting stage is in an open position and vice versa. The sperm is then allowed to enter the space formed by the second, non-closed constriction element. It is stopped there by the third constriction element, which is in a closed position. This operative state can remain for a desired period of time, such as one day. FIG. 593A illustrates the constriction device of FIG. 592 in a third interrupting stage, wherein every other constriction element is in a closed position, exactly as in the first interrupting stage. The sperm shown in FIGS. 593A and 593B, is allowed to enter the space formed by the third, non-closed constriction element. It is stopped there by the fourth constriction element, which is in a closed position. This operative state can remain for a desired period of time, such as one day. Repeating this process, the movement of a sperm can be delayed for a desired period of time until it reaches the other end of the constriction device. Since the life of a sperm is less than about five days, delaying a sperm in this way will ensure that it does not reach an egg in the uterine tube and thereby the constriction device functions to prevent undesired pregnancy. By altering the constricted area of the uterine tube, this will not be harmed like if the same area were constricted for a longer period of time. FIGS. 594A-D show a second embodiment of a constriction device. This operates in a way similar to the first embodiment of a constriction device shown in FIGS. 593A-C. However, in this embodiment, two consecutive constriction elements are in an open position at a time when allowing progress of the sperm.

It will thus be realized that the above described control of sperms in the uterine tube is also applicable to eggs moving through a uterine tube.

### R: Assisting Control for Lubrication of a Synovial Joint

Systems including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described.

The control unit/controller, control systems, communication systems, housing/fixation units and methods could be used for controlling, communicating with and/or operating devices for assisting control of an implantable medical device for lubrication of a synovial joint. Examples of such devices for lubrication of joints will now be described.

Biocompatible material is to be understood as being a material with low level of immune response. Biocompatible materials are sometimes also referred to as biomaterials. Analogous is biocompatible metals a metal with low immune response such as titanium or tantalum. The biocompatible metal could also be a biocompatible alloy comprising at least one biocompatible metal.

Form fitting is to be understood as an element having a part or section which is adapted to enable a mechanical connection of said element to at least one other element using said part or section. Form fitted structure is a structure of an element which enables form fitting.

Elasticity is to be understood as a materials ability to deform in an elastic way.

Elastic deformation is when a material deforms under stress (e.g. external forces), but returns to its original shape when the stress is removed. A more elastic material is to be understood as a material having a lower modulus of elasticity. The elastic modulus of an object is defined as the slope of its stress-strain curve in the elastic deformation region. The elastic modulus is calculated as stress/strain, where stress is the force causing the deformation, divided by the area to which the force is applied; and strain is the ratio of the change caused by the stress.

Stiffness is to be understood as the resistance of an elastic body to deformation by an applied force.

Functional hip movements are to be understood as movements of the hip that at least partly correspond to the natural movements of the hip. On some occasions the natural movements of the hip joint might be somewhat limited or altered after hip joint surgery, which makes the functional hip movements of a hip joint with artificial surfaces somewhat different than the functional hip movements of a natural hip joint.

The functional position of an implantable medical device or prosthesis is the position in which the hip joint can perform functional hip movements.

Functional hip joint is a hip joint that can perform functional hip movements either with or without an implanted medical device or prosthesis.

In the following a detailed description of embodiments will be given. In the drawing figures, like reference numerals designate identical or corresponding elements throughout the several figures. It will be appreciated that these figures are for illustration only and are not in any way restricting the scope. Thus, any references to direction, such as “up” or “down”, are only referring to the directions shown in the figures. Also, any dimensions etc. shown in the figures are for illustration purposes. The functional position or normal functional position, of an implantable medical device or prosthesis is the position in which the hip joint can perform functional hip movements. The final position is to be understood as a functional position in which the medical device needs no further position change. The medical device according to any of the embodiments could comprise at least one material selected from a group consisting of polytetrafluoroethylene (PTFE), perfluoroalkoxy (PFA) and fluorinated ethylene propylene (FEP). It is furthermore conceivable that the material comprises a metal alloy, such as cobalt-chromium-molybdenum or titanium or stainless steel, or polyethylene, such as cross-linked polyethylene or gas sterilized polyethylene. The use of ceramic material is also conceivable, either solely in the contacting surfaces, or in the entire medical device, suitable ceramic materials could be zirconium or zirconium dioxide ceramics or alumina ceramics. The part of the medical device in contact with human bone for fixation of the medical device to human bone could comprise a poorhouse structure which could be a porous micro or nano-structure adapted to promote the growth-in of human bone in the medical device for fixation thereof. The porous structure could be achieved by applying a hydroxy-apatite (HA) coating, or a rough open-pored titanium coating, which could be produced by air plasma spraying, in further embodiments a combination of a rough open-pored titanium coating and a HA top layer is also conceivable. The contacting parts could according to some embodiments be made of a self-lubricated material such as a waxy polymer, such as PTFE, PFA, FEP, PE and UHMWPE, or a powder metallurgy material which could be infused with a lubricant, which is preferably a biocompatible lubricant, such as a Hyaluronic acid derivate. It is also conceivable that the material of contacting parts or surfaces of the medical device herein is adapted to be constantly or intermittently lubricated in accordance with several of the embodiments disclosed herein. In yet other embodiments parts or portions of the medical device could comprise a combination of metal materials and/or carbon fibers and/or boron, a combination of metal and plastic materials, a combination of metal and carbon based material, a combination of carbon and plastic based material, a combination of flexible and stiff materials, a combination of elastic and less elastic materials, Corian or acrylic polymers. FIG. 595a shows the hip joint of a human patient in section. The hip joint comprises a caput femur 5 placed at the very top of collum femur 6 which is the top part of the femoral bone 7. The caput femur is in connection with the acetabulum 8 which is a bowl shaped part of the pelvic bone 9. Both the caput femur surface 10 and the acetabulum surface 11 is covered with articular cartilage 13 which acts as a cushion in the hip joint. In patients with hip joint osteoarthritis, this articular cartilage 13 is abnormally worn down due to a low grade inflammation. The hip joint is surrounded by the hip joint capsule 12 which provides support for the joint and hinders luxation. After conventional hip joint surgery, penetrating the hip joint capsule 12, the capsule 12 is dramatically weakened due to the limited healing possibilities of its ligament tissue. By not having to perform a total hip joint replacement the hip joint capsule 12 can remain intact.

The femoral bone, as well as most other bones in the human body comprises cortical bone, the outer dense, sclerotic bone, and cancellous bone comprised of a less dense cell structure comprising the bone marrow. FIG. 595b shows a cross-section of the collum femur (6 in FIG. 595a) displaying the cortical bone 601 and the cancellous bone 602, the cortical bone 601 thus enclosing the cancellous bone 602. FIG. 596 shows the step of creating a bore in the femoral bone from the lateral side of the thigh using an orthopedic drill 2301. The bore penetrates the most proximal part of the femoral bone, being the caput femur 5 and thus reaches the synovial area of the hip joint comprising the contacting surfaces of caput femur 5 and acetabulum 8, the synovial area being the area in which the synovial fluid is present. FIG. 597 shows the step of inserting an implantable device being a cartridge 2302 comprising a solid lubricant 2303 housed within the walls 2304 of the cartridge 2302. The solid lubricant being adapted to lubricate the contacting surfaces of the acetabulum 8 and the caput femur 5. The bottom portion of the cartridge 2302 comprises a feeding device adapted to feed the solid lubricant, here comprising a spring member 2306 which presses on a bottom plate 2310 disposed within the cartridge 2302, further pressing the solid lubricant 2303 through the cartridge 2302 and in the synovial area for lubricating said contacting surfaces. The spring member 2306 is in contact with the dividing wall 2307 of the cartridge, on the other side of the dividing wall 2307 of the cartridge 2302 a retention member 2308 for retaining the cartridge 2302 in the femoral bone is disposed. The retention member comprises two spring loaded bone engaging members 2311 engaging the inside of the bore when the cartridge 2302 is fully inserted into the bore and thus restrains the cartridge 2302 within the bore (shown in FIG. 598). The solid lubricant could comprise high-molecular weight hyaluronic acid. Hyaluronan is available in different qualities, such as relating to purity, molecular weight and degree of crosslinking. With regard to molecular weight, many different qualities are available, ranging from low molecular weight (LMW) or about 50,000 Da to high molecular weight (HMW) or about 4-6,000,000 Da. An increase in molecular weight results in corresponding increase in viscosity, from an oily liquid to a gel-like semisolid. Suitable sodium hylauronates may in one aspect have a molecular mass of at least 5-6 million before sterilization which when dissolved to a 1% (w/w) solution will obtain similar characteristics as Healon® ophthalmic viscoelastic solution (OVD) (available from Abbot Medical Optics, Inc., Santa Ana, California), or when dissolved to 2.3% (w/w) will resemble Healon® 5′ OVD (available from Abbot Medical Optics, Inc., Santa Ana, California)). The preparation and purification of this type of sodium hyaluronate and to generate viscoelastic solutions are described in more detail in U.S. Pat. Nos. 4,141,973 and 6,086,697. Also high viscosity, high molecular mass sodium hyaluronates such as those described in U.S. Pat. No. 5,681,825 (marketed as viscoelastic under the trade name Healon® GV) can be used with the present invention. One of ordinary skill in the art will realize that, in other aspects of the invention, suitable sodium hylauronates may have a lower molecular mass, as low as 100,000 Da. Clearly, the desired molecular weight is dependent on the class of polymer that is desired to be used in association with the present invention. By way of example, and not of limitation, suitable viscoelastic solutions may be formed using HPMC in the weight range of from about 30,000 to about several hundred thousand daltons. Similarly, suitable viscoelastic solutions may be formed using chondroitin sulphate in the weight range starting from about 20,000 to about 30,000. In general, the molecular weight of the chosen viscoelastic compound (whether it is sodium hyaluronate, HPMC or another viscoelastic) will be selected based on the desired viscoelastic properties of the final solution. The solid lubricant could comprise a crosslinking agent chosen from 1, 2, 3, 4-diepoxybutane, divinyl sulfone further containing a hydrophilic polymer chosen from synthetic and natural polysaccharides, such as hydroxyethyl cellulose, carboxymethyl cellulose, xanthan gum, chondroitin sulfate, heparin, proteins of various types, such as collagen, elastin, albumin, a globulin, etc., or sulfated proteins such as keratin sulfate and sulfated aminoglycosaminoglycans, synthetic water-soluble polymers, such as polyvinyl alcohol and its co-polymers, co-polymers of poly-(hydroxethyl) methacrylate and the like.

FIG. 598 shows the implantable medical device when a portion of the solid lubricant 2303 has been pressed into the synovial area of the hip for lubricating the hip joint. The spring loaded cartridge maintains a pressure in the synovial area and as some of the solid lubricant over time is resorbed by, or diffuses through the encapsulating tissue of the hip joint, new lubricant is added from the portion still housed within the cartridge by means of the spring member 2306. According to one embodiment the solid lubricant has thixotropic or shear thinning properties such that the strain which is placed on the exposed portion of the solid lubricant alters the viscosity of the solid lubricant for creating a well-functioning lubricant. FIG. 599a shows the hip joint in section when all of the solid lubricant in the cartridge 2302 has been pressed into the synovial area of the joint. Depending on the degree of damage on the joint and the particular patient this could take from a number of weeks to several years, after which the cartridge 2302 needs to be replaced or refilled. Further, the process of removing the cartridge 2302 is shown. An incision has been performed on the lateral side of the thigh, beneath the greater trochanter 2312, through which incision the cartridge 2302 can be removed. FIG. 599b shows the placing of a replacement cartridge 2302 into the bore. The replacement cartridge 2302 carries more solid lubricant 2303 adapted to lubricate the joint surfaces of the hip joint. FIGS. 596-606 shows an embodiment in which the principle disclosed with reference to FIGS. 596-599 is applied to the knee of a patient. FIG. 600 shows the creation of a bore in the distal part of the femoral bone 7 using an orthopedic drill 2301. The bore is created through an incision from the posterior side of the femoral bone 7. The bore reaches the surfaces of the knee joint, i.e. the distal surface of the femoral bone 5 and the proximal surface of the tibia bone 40. FIG. 602 shows the insertion of a cartridge 2302, according to any of the embodiments disclosed herein, in the bore created in the distal portion of the femoral bone 7. FIG. 603 shows the removal of the cartridge from the bore in the femoral bone 7, after the cartridge 2302 has been used such that little or no solid lubricant 2303 remains in the cartridge 2302. FIG. 604 shows the placing of a replacement cartridge 2302 into the bore. The replacement cartridge 2302 carries more solid lubricant 2303 adapted to lubricate the joint surfaces of the knee joint. FIG. 605 shows the cartridge 2302 when inserted into a prosthetic hip joint surface 41 adapted to replace the contacting surface of the femoral bone 7. The medical device here being adapted to lubricate the surface between the prosthetic hip joint surface 41 and the natural hip joint surface of the tibia bone or another prosthetic surface adapted to replace the tibia 40 contacting surface. FIG. 606 shows the femoral bone 7 and tibia bone 40 in a frontal view. Two bores 42a,b have been made in the femoral bone 7 and one bore 42c has been made in the tibia bone. These bores are examples of different locations in which the medical devices can be placed. In some embodiments in is further conceivable that several medical devices are needed to lubricate one joint. FIG. 607 shows an embodiment in which the cartridge 2302, according to any of the embodiments herein, containing the solid lubricant 2303, is placed in a bore in the humerus bone 45 for lubricating the shoulder joint of a patient using the solid lubricant. FIG. 608 shows the hip joint in section in an embodiment in which a bore is created in the pelvic bone 9 by means of an orthopedic drill 2301. FIG. 609 shows the placing of a cartridge 2302 containing solid lubricant 2303 in the pelvic bone 9. The solid lubricant 2303 is in accordance with the other embodiments disclosed herein adapted to lubricate the surfaces of acetabulum 8 and the caput femur 5. FIG. 610 shows the implantable medical device when a portion of the solid lubricant 2303 has been pressed into the synovial area of the hip for lubricating the hip joint. The spring loaded cartridge 2302 maintains a pressure in the synovial area and as some of the solid lubricant over time is resorbed by, or diffuses through the encapsulating tissue of the hip joint, new lubricant is added from the portion still housed within the cartridge by means of the spring member 2306. According to one embodiment the solid lubricant has thixotropic or shear thinning properties such that the strain which is placed on the exposed portion of the solid lubricant alters the viscosity of the solid lubricant for creating a well-functioning lubricant. FIG. 611 shows the hip joint in section when the all of the solid lubricant in the cartridge 2302 has been pressed into the synovial area of the joint. Depending on the degree of damage on the joint and the particular patient this could take from a number of weeks to several years, after which the cartridge 2302 needs to be replaced or refilled. In FIG. 611 the process of removing the cartridge 2302 through the abdominal cavity is shown. FIG. 612 shows the placing of a replacement cartridge 2302 into the bore. The replacement cartridge 2302 carries more solid lubricant 2303 adapted to lubricate the joint surfaces of the hip joint. FIGS. 613a-617e shows the cartridges in further detail. FIG. 613a shows an embodiment in which the cartridge comprises an ejection device 2340 for affecting the retention member comprising a resilient membrane 2351 affecting to spring loaded members 2352 which in turn affects bone engaging members 2311. In normal operation the bone engaging members 2311 makes sure that the cartridge remains secured in the bore, and when the resilient member is pressed the bone engaging members 2311 folds such that the cartridge can be removed from the bore. FIG. 613b shows an alternative embodiment of the retention member comprising bone engaging members 2353, in which the bone engaging members 2353 are adapted to be placed in a grove in the bore for retaining the cartridge inside the bore. The alternative bone engaging members 2353 are spring loaded in radial direction. FIG. 613c shows an alternative embodiment in which the feeding device comprises elastic members 2354, preferably made from elastic material, propels the bottom plate 2310 feeding the solid lubricant 2303, out of the cartridge. FIG. 613d shows the medical device according to an embodiment comprising a feeding device in which the bottom plate 2310 is propelled by an energized operation device, such as an electric motor 2355. The electric motor is connected to the bottom plate 2310 by means of a threaded member 2356 engaging a corresponding threaded part of the motor 2355. FIG. 613e shows an embodiment of a prosthetic part 2360 adapted to be fixated to the femoral bone and replace the contacting surface of the caput femur. The prosthetic part 2360 is adapted to receive the medical device according to any of the embodiments herein in a bore 42 in the prosthetic part 2360. The prosthetic part 2360 comprises a groove 2358 in the bore 42 adapted to receive the bone engaging members 2353 according to the embodiment disclosed in FIG. 17b for retaining the medical device or cartridge in the bore 42. In embodiments where the medical device comprises an energized unit, such as the motor 2355 disclosed with reference to FIG. 613d a unit for powering and/or controlling the medical device could be necessary, an example of such a unit suitable for this purpose will now be disclosed. The unit is preferably connected to the medical device by means of electrical leads and/or hydraulic conduits. The term “functional parts” is to be interpreted as all parts of the control assembly for the electrical or hydraulic operation of the assembly. FIG. 614 shows the body of a human patient 1. A control assembly 1110 adapted for controlling an implanted medical device is shown subcutaneously implanted in the abdominal area of the patient's body. Although a specific position for the control assembly is shown in the figure, it will be appreciated that the control assembly can be provided essentially anywhere in the patient's body, preferably relatively close to the implanted medical device which it is adapted to control. Generally speaking, the control assembly 1110 may be placed in the abdomen, thorax, muscle fascia (e.g. in the abdominal wall), subcutaneously, or at any other suitable location. An overall side view of the control assembly 1110 is shown in FIG. 615. The control assembly comprises a first unit 120 subcutaneously implanted at a first side of a body tissue 103 in the patient, such as the rectus abdominis muscle running vertically on each side of the anterior wall of the human abdomen. In other words, the first unit is positioned between the skin 105 of the patient and the body tissue 103. A second unit 130 is implanted in a body cavity 107 of the patient at a second side of the body tissue 103, i.e., that the side opposite of the side at which the first unit 120 is provided. The first 120 and/or second units 130 preferably have circular or elliptical cross-sectional shape when viewed from outside the patient's body, see FIGS. 616a, 616b, showing a top view of the assembly having elliptical and circular shape, respectively. Combined with a smoothly curved sectional shape, this avoids any sharp corners on the units 120, 130, which could cause injuries to the patient in which the control assembly 1110 is implanted. An interconnecting device 140 constitutes a mechanical interconnection between the first and second units 120, 130 so that the assembly 1110 is kept in place by the body tissue 103. The interconnecting device has a cross-sectional area which is smaller than the cross-sectional area of the first unit and the second unit in a plane parallel to the extension of the body tissue. In this way, a hole 103a in the body tissue 103 through which the interconnecting device 140 extends can be sufficiently small so that it is avoided that one or the other of the units 120, 130 “slips through” the body tissue 103. Also, the cross-sectional shape of the interconnecting device 140 is preferably circular so as to avoid damage to the body tissue 103. The interconnection device 140 can be integral with one of the first and second units 120, 130. Alternatively, the interconnection device 140 is a separate part, which is connected to the first and second units 120, 130 during implantation of the control assembly 1110. In a preferred embodiment, the interconnection device 140 is hollow so as to house various wires, hoses etc. electrically or hydraulically interconnecting the first and second devices 120, 130. Alternatively or additionally, the interconnection device 140 is made of an elastic material, such as rubber, so that the control assembly 1110 can adapt to the movements of the patient in which it is implanted. The control assembly 1110 is adapted to control a powered implanted medical device 100, see FIG. 617. The implanted medical device can be any kind of powered operation device, such as a hydraulically, pneumatically or mechanically powered operation device, such as the motor disclosed with reference to FIG. 613d. The medical device 100 can be any kind of implant, such as a constriction device adapted to constrict and release a bodily lumen or vessel, a stimulation device adapted to electrically stimulate a bodily organ, an inflatable device adapted to fill for example the corpora cavernosa of the patient etc. The implanted medical device is preferably very small, having a diameter of less than 5 centimeters, to fit in the different target areas of the body. Depending on the kind of power required to control the medical device 100, an interconnection 102 in the form of an electrical wire, a pneumatic hose etc., is provided between the control assembly 1110 and the medical device 100. The control unit 1110 is adapted to receive energy, preferably wireless energy, transmitted from an external energy source or energizer 110 located outside the skin in the vicinity of the control unit 1110. The energizer 110, which is an external device which functions as the charging equipment and control device for the control assembly, is connected via a connection, such as a serial RS232 connection, to a computer 112, such as a standard desktop PC or a laptop computer. The PC software implements the user interface to the implant system, and function as the control unit and read back unit of the implant system. A block diagram of the implant system is shown in FIG. 618. Energy is transferred by means of the wireless coupling between an energizer coil 110a forming part of the energizer 110 and a control assembly coil 10a forming part of the control assembly 1110. Similarly, control information is transferred between the energizer 110 by means of a wireless communications interface comprising an energizer antenna 110b forming part of the energizer 110 and a control assembly antenna 10b forming part of the control assembly 1110. In this way, both energy and communication information can be transferred wirelessly to and from the control assembly 1110. Although separate devices are shown for transfer of energy and information, i.e., the coils and the antennas, respectively, it will be appreciated that the coils 10a, 110a can be implemented for use as an antenna as well, whereby control information can be transferred by means of the coils and no separate antennas are needed for that purpose.

The functional parts of the control assembly 1110 can be provided either in the first unit 120 or in the second unit 130 or in both the first and the second unit. In other words, at least one of the first and the second unit is adapted to control a powered implanted medical device. FIG. 619 is a sectional view of the control assembly 1110 showing an example of the contents of the first unit 120, the second unit 130 and the interconnection device 140. It is also shown that the interconnection device 140 is provided integral with the first unit 120, forming an extension from the central portion of the first unit. The outer end of the extension is provided with barbs 142 engaging the rim of a hole 122 provided in the central portion of the second unit. In this way, the control assembly 1110 can be assembled by a simple snap-together operation, as will be described in more detail below. A coil 150 is provided in the first unit, the coil being an energy transfer coil arranged to pick up wireless electro-magnetic energy and signals from an external unit. The number of rounds in the coil is adapted for the specific operation and is preferably at least ten. The end portions of the coil 150 extend perpendicularly to the general extension of the coil and are lead through the hollow interconnection device 140 to be connected to the functional parts provided in the second unit 130, shown as a block diagram in FIG. 620. The functional parts shown in this figure is a non-limiting example of the different parts comprised in a control assembly according to the invention. A micro controller unit (MCU) 152 is provided as a main controller unit of the control assembly 1110 and it thus provided with control software for controlling the operation of the functional parts of the control assembly. In a preferred embodiment, this MCU is a Texas Instruments MSP430F149 MCU. Although not shown in the figure, the MCU can be supplemented by additional peripheral circuits, such as a gate array implemented as an application specific integrated circuit (ASIC), acting as an interface to the various functional parts. The MCU 152 receives commands from the energizer 110 via a wireless communication link, see below, and takes decision about actions. The MCU 152 thus supervises all functions in the control assembly 1110. The MCU stores application specific parameters and calibration data in an external EEPROM (not shown). The main functionality of the control assembly 1110 is that all operations, such as stimuli, adjustments or measurements are initiated via the energizer 110. Thus, the energizer has two main functions: User interface via RF communication with the control assembly 1110 and energy transfer to the control assembly. The control assembly 1110 can be OFF or in Standby when “unconnected”. All functions within the control assembly are controlled via the wireless communication link. The energy transfer function runs in the background as soon as the user has initiated a charge operation. The coupling between the energizer and the receiver coil is displayed by means of a graphical user interface (GUI) on the display of the energizer 110. If the communication is interrupted under operation, the active function is terminated with a warning message. As soon as correct connection is obtained the last function can be re-activated from the GUI. The MCU 152 is connected to a charge control unit 154, which in turn is connected to the coil 150, which in this embodiment is provided in the first unit 120. The charge control unit comprises an energy storage capacitor 154a, from which the normal power supply is taken. In the preferred embodiment, the energy storage capacitor 154a has a value of at least 300 Farad with a maximum voltage of 2.3V. The energy storage capacitor is normally connected to the energy transfer coil 150, preventing hazardous voltages to occur at the supply circuits. The voltage from the energy transfer coil 150 is rectified by means of half-wave rectification. The transferred energy is set by the voltage on the energizer coil 110a, see FIG. 618, and the geometric placement relative the energy transfer coil 150 on the control assembly. The leakage inductances make the behavior of a current generator, that is, the voltage across the energy storage capacitor 154a will have a very little influence on the current. The charge function is controlled from the energizer software, which depends on current and voltage readings on the reservoir capacitor. The applied energy transfer will charge the capacitor up to a limit voltage, which in the preferred embodiment is 2.3V, while the charge current preferably is limited to 2A by the energizer design. If the energy storage capacitor energy drops below a lower limit voltage, in the preferred embodiment 1.2V, MCU 152 will be notified to terminate any activity and go to STAND-BY mode. An over voltage protection will disconnect the receiver inductor if the energy storage capacitor voltage reaches 2.35V. All functional parts of the control assembly will still be supplied from the capacitor and a battery charge process will continue. Thus, the voltage will vary between 1.0 and 2.3V dependent of the charge status. This voltage feeds a switch converter for supplying the MCU including any gate array. It is preferred that the gate array supply may be shut down by the MCU to save energy. The control assembly shall be functional for 36 hours relying on the capacitor only. A chargeable battery 154b is also provided as part of the charge control unit 154. The capacity of the battery is preferably approximately ten times that of the energy storage capacitor 154a. In the preferred embodiment, the battery used is three 1.2 V batteries, such as Varta V500-HT, connected in series. This gives a nominal voltage of 3.6V. The battery management consists of two main activities: Charging and de-charging (transfer energy to the reservoir capacitor. Normally the battery is unused and energy is supplied from the capacitor. A battery charging functionality is implemented in hardware with supervision and control from the MCU 52. The chargeable battery is charged from the energy storage capacitor 154a when the voltage across the energy storage capacitor exceeds 1.9V. This limit will prevent the battery charger from emptying the capacitor energy When the voltage across the energy storage capacitor is less than 1.3V, the battery will charge the energy storage capacitor a constant current by means of a step-down converter (not shown). The charge current is in the preferred embodiment 350 mA with dv/dt detection. Temperature supervision will turn off any charge operation if the battery temperature increases more than 0.7 degrees per min. The energy transfer is controlled from the software in the computer 112. The MCU 52 will read the voltage and current on the energy storage capacitor 154a. The values are then on command transmitted to the computer 112, which controls the energizer. If the energy storage capacitor 154a has a 300F capacitance and the charge current is normally well below 2A, the voltage changes will be very slow—minutes for a 0.1V increase. This slow behavior makes an ordinary PI-regulator superfluous. The preferred embodiment is an on/off regulator with a 100 mV hysteresis gap. At the very startup when there may be no energy in the capacitor. A special bypass power will turn on the MCU/transceiver. Thus the feedback communication system will be active almost immediately when the energizer coil is applied. The control assembly 1110 can be in four different power modes, namely:
OFF: All circuits are turned off. The transceiver 156 is powered from battery 54b, but in sleep mode.
WAKE-UP: The power is fee from the energy transfer coil 150, unconditionally of the status of the capacitor 154a or the battery 154b. This makes the control assembly to respond immediately when the energizer is applied.
STAND-BY: MCU active but no stimuli, sensor or motor voltage active.
ACTIVE: The MCU in operation. Motor/Sensors/Stimuli etc. active

The mode is controlled by the software in the MCU.

The MCU 152 communicates with the energizer by means of the antenna 10b, see FIG. 618, which is electrically connected to a transceiver 156 in the control assembly 1110. The transceiver 156 is in turn connected to the MCU 152. The transceiver 156 can be any suitable transceiver circuit and is in the described embodiment a circuit marketed under the name ZL70101 by Zarlink Semiconductor Inc. This provides RF communication using the MICS standard. The transceiver preferably uses a serial peripheral interface (SPI) to communicate with the MCU and is specified for 2.1-3.6V supply. The transceiver needs to be under continuous power but have a low power mode with very low quiescent current where it can be woken up by using either by toggling a wakeup input or alternatively by MICS band or 2.4 GHz radio signals. In the preferred embodiment, the antenna 10b is adapted to support MICS telemetry that operates in the dedicated 402-405 MHz band. The most probable implementation of the transceiver 156 will use a system that can be implemented using also a secondary 2.4 GHz ISM band for wake up purposes, which will then also require attention to safeguard antenna functionality also at these frequencies. The wake up antenna is assumed to be identical to the MICS antenna since alternate solutions would require separate hermetic feed-through connections that adds considerable costs. The 2.4 GHz aspect of the antenna is an electrically large antenna that works well with most MICS antenna implementations. One temperature sensor will be used for sensing the temperature of the battery and one sensing the encapsulation. It is also possible to connect one or more external temperature sensors. The sensor accuracy is typically +/−0.5 degrees between −30-+70 degrees and better than +/−0.25 degrees between 20-45 degrees.

One or more pressure sensors 160 are connected to an A/D input of the MCU 52. The pressure sensors preferably have a sensing range of 0-2 bars. The sensors can be of the SMD 3000 series type 3SC-0500-5212 from Merit Sensor Systems.

One or more motors can be connected to the control assembly 1110. The operation of these motors is controlled by means of a respective motor controller. In a preferred embodiment, this motor controller consists of 5 H-bridge with current measurement and rotation sensing. The control options are forward, backward, or break. The control is either ON or OFF, i.e., no speed control is implemented. Alternatively, speed control can be implemented, such as by means of a pulse width modulated (PWM) signal.

In order to conserve power, a select signal to each motors current feedback needs to be activated before any measurements can be done.

The current through the motor is measured in order to differentiate four states:
Normal running operation
Motor stall
Motor short-circuit/open circuit
Slipping of magnetic clutch

Different mechanics and motors will have different thresholds for the states. This will be evaluated by software.

The rotation of the motors will be monitored either by an internal encoder in the motor or by external sensors/encoders. The sensing of the movement can be done with a low power Hall element, for example Allegro A139X series, in combination with a comparator that sets the sensitivity or by optical encoders depending on the mechanics. There are two sensors for each motor to be able to determine both speed and direction. End switches can optionally be provided.

Depending on the mechanics and the motors different rotation sensing methods can be used. Exact trip points and hysteresis are application dependent. It should be noted that the mentioned sensors are merely examples and that more types can be added.

Sensing on outgoing axle can be used when there is no encoder on the motor. The rotation sensing can be done with two Hall-effect sensors, such as A1391SE sensors from Allegro MicroSystems, Inc.. By using two sensors per motor both direction and speed can be determined. The phase between the detectors shall be 90 degrees, which is set by the mechanical mounting of the devices.

Alternatively, a reflex detector can be used for rotation sensing.

In yet an alternative embodiment, an integrated encoder in the motor can be used for rotation determination.

The control assembly can be adapted to control the operation of an implanted medical device in the form of one or more electrodes used to electrically stimulate an organ in the patient' body, such as the corpora cavernosa or crura or the prolongations thereof of a male patient's penile tissue, the colon, rectum, or anus or the prolongation thereof, the urine bladder, the urethra or the prolongation thereof, or any other bodily lumen which requires electrical stimulation under control of the patient or a doctor.

The stimuli generators 164 are designed around a high speed, high current output operational amplifiers, such as the AD825 from Analog Devices, Inc. Each output has a dc current blocking capacitor. A DC servo prevents the capacitor to charge due to offset current errors In one embodiment, the implanted medical device contains 4+4 electrodes to which a constant current pulse generator is connected. The current generator can be connected to two or several electrodes simultaneously.

The current pulses always consist of one positive current pulse and one negative current pulse in any order. The stimuli pulses are configurable regarding current amplitude; pulse widths, number of pulses in a pulse train and pulse frequency. The control assembly 1110 ensures that the pulses are charged balanced.

The software of the computer 112 is adapted to write configuration parameters to the control assembly 1110 and to start and stop stimulation with those parameters. The stimulation can “move” between different electrodes to e.g. create an artificial peristalsis. In a preferred embodiment, the stimuli amplitude is be up to 20 mA with +/−14V available. One or more capacitance measuring inputs are provided for determination of a physical or mechanical position. The input has a working range of 5-100 pF. The motion sensor is a piezo polymer strip that generates a charge/voltage during movement of an intestine. Each motion sensor is adjusted depending on the application in order to apply an appropriate gain. The first unit 120 could comprises an injection port 170 adapted to receive an injection needle. The injection port comprises a reservoir with a silicone septum. Fluid is added to or removed from the interior reservoir of the first unit 120 by inserting a Huber needle percutaneously into the septum. Although the septum is made of silicone, the means of the injection port for receiving a needle includes any structure configured to self-seal after puncture with a non-coring needle. Different systems comprising a control unit for controlling the implanted lubricating system will now be described with reference to FIGS. 666-690.

FIG. 666 illustrates a system for treating a disease comprising an apparatus 10 in the form of a system for lubrication. An implanted energy-transforming device 1002 is adapted to supply energy consuming components of the apparatus with energy via a power supply line 1003. An external energy-transmission device 200 for non-invasively energizing the apparatus 10 transmits energy by at least one wireless energy signal. The implanted energy-transforming device 1002 transforms energy from the wireless energy signal into electric energy which is supplied via the power supply line 1003. The implanted energy-transforming device 1002 may also comprise other components, such as: a coil for reception and/or transmission of signals and energy, an antenna for reception and/or transmission of signals, a microcontroller, a charge control unit, optionally comprising an energy storage, such as a capacitor, one or more sensors, such as temperature sensor, pressure sensor, position sensor, motion sensor etc., a transceiver, a motor, optionally including a motor controller, a pump, and other parts for controlling the operation of a medical implant. The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field. The wireless energy-transmission device 200 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal. Generally speaking, the energy-transforming device 1002 is provided for transforming wireless energy of a first form transmitted by the energy-transmission device 200 into energy of a second form, which typically is different from the energy of the first form. The implanted apparatus 10 is operable in response to the energy of the second form. The energy-transforming device 1002 may directly power the apparatus with the second form energy, as the energy-transforming device 1002 transforms the first form energy transmitted by the energy-transmission device 200 into the second form energy. The system may further include an implantable accumulator, wherein the second form energy is used at least partly to charge the accumulator. Alternatively, the wireless energy transmitted by the energy-transmission device 200 may be used to directly power the apparatus, as the wireless energy is being transmitted by the energy-transmission device 200. Where the system comprises an operation device for operating the apparatus, as will be described below, the wireless energy transmitted by the energy-transmission device 200 may be used to directly power the operation device to create kinetic energy for the operation of the apparatus. The wireless energy of the first form may comprise sound waves and the energy-transforming device 1002 may include a piezo-electric element for transforming the sound waves into electric energy. The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus. Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal. The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus. The external energy-transmission device 200 also includes a wireless remote control having an external signal transmitter for transmitting a wireless control signal for non-invasively controlling the apparatus. The control signal is received by an implanted signal receiver which may be incorporated in the implanted energy-transforming device 1002 or be separate there from. The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field. The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals. FIG. 667 illustrates the system of FIG. E34 in the form of a more generalized block diagram showing the apparatus 10, the energy-transforming device 1002 powering the apparatus 10 via power supply line 1003, and the external energy-transmission device 200, The patient's skin SK, generally shown by a vertical line, separates the interior of the patient to the right of the line from the exterior to the left of the line. FIG. 668 shows an embodiment identical to that of FIG. 667, except that a reversing device in the form of an electric switch 1006 operable for example by polarized energy also is implanted in the patient for reversing the apparatus 10. When the switch is operated by polarized energy the wireless remote control of the external energy-transmission device 200 transmits a wireless signal that carries polarized energy and the implanted energy-transforming device 1002 transforms the wireless polarized energy into a polarized current for operating the electric switch 1006. When the polarity of the current is shifted by the implanted energy-transforming device 1002 the electric switch 1006 reverses the function performed by the apparatus 10. FIG. 669 shows an embodiment identical to that of FIG. 667, except that an operation device 1007 implanted in the patient for operating the apparatus 10 is provided between the implanted energy-transforming device 1002 and the apparatus 10. This operation device can be in the form of a motor 1007, such as an electric servomotor. The motor 1007 is powered with energy from the implanted energy-transforming device 1002, as the remote control of the external energy-transmission device 200 transmits a wireless signal to the receiver of the implanted energy-transforming device 1002. FIG. 670 shows an embodiment identical to that of FIG. 667, except that it also comprises an operation device in the form of an assembly 1008 including a motor/pump unit 1009 and a fluid reservoir 1010 is implanted in the patient. In this case the apparatus 10 is hydraulically operated, i.e. hydraulic fluid is pumped by the motor/pump unit 1009 from the fluid reservoir 1010 through a conduit 1011 to the apparatus 10 to operate the apparatus, and hydraulic fluid is pumped by the motor/pump unit 1009 back from the apparatus 10 to the fluid reservoir 1010 to return the apparatus to a starting position. The implanted energy-transforming device 1002 transforms wireless energy into a current, for example a polarized current, for powering the motor/pump unit 1009 via an electric power supply line 1012. Instead of a hydraulically operated apparatus 10, it is also envisaged that the operation device comprises a pneumatic operation device. In this case, the hydraulic fluid can be pressurized air to be used for regulation and the fluid reservoir is replaced by an air chamber. In all of these embodiments the energy-transforming device 1002 may include a rechargeable accumulator like a battery or a capacitor to be charged by the wireless energy and supplies energy for any energy consuming part of the system. As an alternative, the wireless remote control described above may be replaced by manual control of any implanted part to make contact with by the patient's hand most likely indirect, for example a press button placed under the skin. FIG. 671 shows an embodiment comprising the external energy-transmission device 200 with its wireless remote control, the apparatus 10, in this case hydraulically operated, and the implanted energy-transforming device 1002, and further comprising a hydraulic fluid reservoir 1013, a motor/pump unit 1009 and a reversing device in the form of a hydraulic valve shifting device 1014, all implanted in the patient. Of course, the hydraulic operation could easily be performed by just changing the pumping direction and the hydraulic valve may therefore be omitted. The remote control may be a device separated from the external energy-transmission device or included in the same. The motor of the motor/pump unit 1009 is an electric motor. In response to a control signal from the wireless remote control of the external energy-transmission device 200, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 10. The remote control of the external energy-transmission device 200 controls the hydraulic valve shifting device 1014 to shift the hydraulic fluid flow direction between one direction in which the fluid is pumped by the motor/pump unit 1009 from the hydraulic fluid reservoir 1013 to the apparatus 10 to operate the apparatus, and another opposite direction in which the fluid is pumped by the motor/pump unit 1009 back from the apparatus 10 to the hydraulic fluid reservoir 1013 to return the apparatus to a starting position. FIG. 672 shows an embodiment comprising the external energy-transmission device 200 with its wireless remote control, the apparatus 10, the implanted energy-transforming device 1002, an implanted internal control unit 102 controlled by the wireless remote control of the external energy-transmission device 200, an implanted accumulator 1016 and an implanted capacitor 1017. The internal control unit 102 arranges storage of electric energy received from the implanted energy-transforming device 1002 in the accumulator 1016, which supplies energy to the apparatus 10. In response to a control signal from the wireless remote control of the external energy-transmission device 200, the internal control unit 102 either releases electric energy from the accumulator 1016 and transfers the released energy via power lines 1018 and 1019, or directly transfers electric energy from the implanted energy-transforming device 1002 via a power line 1020, the capacitor 1017, which stabilizes the electric current, a power line 1021 and the power line 1019, for the operation of the apparatus 10. The internal control unit is preferably programmable from outside the patient's body. In a preferred embodiment, the internal control unit is programmed to regulate the apparatus 10 according to a pre-programmed time-schedule or to input from any sensor sensing any possible physical parameter of the patient or any functional parameter of the system. In accordance with an alternative, the capacitor 1017 in the embodiment of FIG. 672, 10 may be omitted. In accordance with another alternative, the accumulator 1016 in this embodiment may be omitted. FIG. 673 shows an embodiment identical to that of FIG. 667, except that a battery 1022 for supplying energy for the operation of the apparatus 10 and an electric switch 1023 for switching the operation of the apparatus 10 also are implanted in the patient. The electric switch 1023 may be controlled by the remote control and may also be operated by the energy supplied by the implanted energy-transforming device 1002 to switch from an off mode, in which the battery 1022 is not in use, to an on mode, in which the battery 1022 supplies energy for the operation of the apparatus 10. FIG. 674 shows an embodiment identical to that of FIG. 673, except that an internal control unit 102 controllable by the wireless remote control of the external energy-transmission device 200 also is implanted in the patient. In this case, the electric switch 1023 is operated by the energy supplied by the implanted energy-transforming device 1002 to switch from an off mode, in which the wireless remote control is prevented from controlling the internal control unit 102 and the battery is not in use, to a standby mode, in which the remote control is permitted to control the internal control unit 102 to release electric energy from the battery 1022 for the operation of the apparatus 10. FIG. 675 shows an embodiment identical to that of FIG. 674, except that an accumulator 1016 is substituted for the battery 1022 and the implanted components are interconnected differently. In this case, the accumulator 1016 stores energy from the implanted energy-transforming device 1002. In response to a control signal from the wireless remote control of the external energy-transmission device 200, the internal control unit 102 controls the electric switch 1023 to switch from an off mode, in which the accumulator 1016 is not in use, to an on mode, in which the accumulator 1016 supplies energy for the operation of the apparatus 10. The accumulator may be combined with or replaced by a capacitor. FIG. 676 shows an embodiment identical to that of FIG. 675, except that a battery 1022 also is implanted in the patient and the implanted components are interconnected differently. In response to a control signal from the wireless remote control of the external energy-transmission device 200, the internal control unit 102 controls the accumulator 1016 to deliver energy for operating the electric switch 1023 to switch from an off mode, in which the battery 1022 is not in use, to an on mode, in which the battery 1022 supplies electric energy for the operation of the apparatus 10. Alternatively, the electric switch 1023 may be operated by energy supplied by the accumulator 1016 to switch from an off mode, in which the wireless remote control is prevented from controlling the battery 1022 to supply electric energy and is not in use, to a standby mode, in which the wireless remote control is permitted to control the battery 1022 to supply electric energy for the operation of the apparatus 10. It should be understood that the switch 1023 and all other switches in this application should be interpreted in its broadest embodiment. This means a transistor, MCU, MCPU, ASIC, FPGA or a DA converter or any other electronic component or circuit that may switch the power on and off. Preferably the switch is controlled from outside the body, or alternatively by an implanted internal control unit. FIG. 677 shows an embodiment identical to that of FIG. 673, except that a motor 1007, a mechanical reversing device in the form of a gear box 1024, and an internal control unit 102 for controlling the gear box 1024 also are implanted in the patient. The internal control unit 102 controls the gear box 1024 to reverse the function performed by the apparatus 10 (mechanically operated). Even simpler is to switch the direction of the motor electronically. The gear box interpreted in its broadest embodiment may stand for a servo arrangement saving force for the operation device in favor of longer stroke to act. FIG. 678 shows an embodiment identical to that of FIG. 684 except that the implanted components are interconnected differently. Thus, in this case the internal control unit 102 is powered by the battery 1022 when the accumulator 1016, suitably a capacitor, activates the electric switch 1023 to switch to an on mode. When the electric switch 1023 is in its on mode the internal control unit 102 is permitted to control the battery 1022 to supply, or not supply, energy for the operation of the apparatus 10. FIG. 679 schematically shows conceivable combinations of implanted components of the apparatus for achieving various communication options. Basically, there are the apparatus 10, the internal control unit 102, motor or pump unit 1009, and the external energy-transmission device 200 including the external wireless remote control. As already described above the wireless remote control transmits a control signal which is received by the internal control unit 102, which in turn controls the various implanted components of the apparatus. A feedback device, preferably comprising a sensor or measuring device 1025, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow. The feedback may be sent to the internal control unit or out to an external control unit preferably via the internal control unit. Feedback may be sent out from the body via the energy transfer system or a separate communication system with receiver and transmitters. The internal control unit 102, or alternatively the external wireless remote control of the external energy-transmission device 200, may control the apparatus 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 102 may comprise a signal receiver or transceiver. Alternatively, the wireless remote control may comprise a signal receiver or transceiver and the internal control unit 102 may comprise a signal transmitter or transceiver. The above transceivers, transmitters and receivers may be used for sending information or data related to the apparatus 10 from inside the patient's body to the outside thereof. Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly. FIG. 680 shows an alternative embodiment wherein the apparatus 10 is regulated from outside the patient's body. The system 100 comprises a battery 1022 connected to the apparatus 10 via a subcutaneous electric switch 1026. Thus, the regulation of the apparatus 10 is performed non-invasively by manually pressing the subcutaneous switch, whereby the operation of the apparatus 10 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed. FIG. 681 shows an alternative embodiment, wherein the system 100 comprises a hydraulic fluid reservoir 1013 hydraulically connected to the apparatus. Non-invasive regulation is performed by manually pressing the hydraulic reservoir connected to the apparatus. Alternatively, the hydraulic fluid reservoir 1013 is adapted to work with an injection port for the injection of hydraulic fluid, preferably for calibration of hydraulic fluid. The system may include an external data communicator and an implantable internal data communicator communicating with the external data communicator. The internal communicator feeds data related to the apparatus or the patient to the external data communicator and/or the external data communicator feeds data to the internal data communicator. FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 1002 connected to implanted energy consuming components of the apparatus 10. Such an energy receiver 1002 may include an energy source and/or an energy-transforming device. Briefly described, wireless energy is transmitted from an external energy source 200a located outside the patient and is received by the internal energy receiver 1002 located inside the patient. The internal energy receiver is adapted to directly or indirectly supply received energy to the energy consuming components of the apparatus 10 via a switch 1026. An energy balance is determined between the energy received by the internal energy receiver 1002 and the energy used for the apparatus 10, and the transmission of wireless energy is then controlled based on the determined energy balance. The energy balance thus provides an accurate indication of the correct amount of energy needed, which is sufficient to operate the apparatus 10 properly, but without causing undue temperature rise. In FIG. 682 the patient's skin is indicated by a vertical line SK. Here, the energy receiver comprises an energy-transforming device 1002 located inside the patient, preferably just beneath the patient's skin SK. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy-source 200a provided in an external energy-transmission device 200 located outside the patient's skin SK in the vicinity of the implanted energy-transforming device 1002. As is well known in the art, the wireless energy E may generally be transferred by means of any suitable Transcutaneous Energy Transfer (TET) device, such as a device including a primary coil arranged in the external energy source 200a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used. The amount of energy received by the implanted energy receiver may be compared with the energy used by the implanted components of the apparatus. The term “energy used” is then understood to include also energy stored by implanted components of the apparatus. A control device includes an external control unit 200b that controls the external energy source 200a based on the determined energy balance to regulate the amount of transferred energy. In order to transfer the correct amount of energy, the energy balance and the required amount of energy is determined by means of a determination device including an implanted internal control unit 102 connected between the switch 1026 and the apparatus 10. The internal control unit 102 may thus be arranged to receive various measurements obtained by suitable sensors or the like, not shown, measuring certain characteristics of the apparatus 10, somehow reflecting the required amount of energy needed for proper operation of the apparatus 10. Moreover, the current condition of the patient may also be detected by means of suitable measuring devices or sensors, in order to provide parameters reflecting the patient's condition. Hence, such characteristics and/or parameters may be related to the current state of the apparatus 10, such as power consumption, operational mode and temperature, as well as the patient's condition reflected by parameters such as; body temperature, blood pressure, heartbeats and breathing. Other kinds of physical parameters of the patient and functional parameters of the device are described elsewhere. Furthermore, an energy source in the form of an accumulator 1016 may optionally be connected to the implanted energy-transforming device 1002 via the control unit 102 for accumulating received energy for later use by the apparatus 10. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 10, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics. For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition. Thus, the internal control unit 102 of the determination device is adapted to determine the energy balance and/or the currently required amount of energy, (either energy per time unit or accumulated energy) based on measurements made by the above-mentioned sensors or measuring devices of the apparatus 10, or the patient, or an implanted energy source if used, or any combination thereof. The internal control unit 102 is further connected to an internal signal transmitter 1027, arranged to transmit a control signal reflecting the determined required amount of energy, to an external signal receiver 200c connected to the external control unit 200b. The amount of energy transmitted from the external energy source 200a may then be regulated in response to the received control signal. Alternatively, the determination device may include the external control unit 200b. In this alternative, sensor measurements can be transmitted directly to the external control unit 200b wherein the energy balance and/or the currently required amount of energy can be determined by the external control unit 200b, thus integrating the above-described function of the internal control unit 102 in the external control unit 200b. In that case, the internal control unit 102 can be omitted and the sensor measurements are supplied directly to the internal signal transmitter 1027 which sends the measurements over to the external signal receiver 200c and the external control unit 200b. The energy balance and the currently required amount of energy can then be determined by the external control unit 200b based on those sensor measurements. Hence, the present solution according to the arrangement of FIG. 682 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus. The internal signal transmitter 1027 and the external signal receiver 200c may be implemented as separate units using suitable signal transfer means, such as radio, IR (Infrared) or ultrasonic signals. Alternatively, the internal signal transmitter 1027 and the external signal receiver 200c may be integrated in the implanted energy-transforming device 1002 and the external energy source 200a, respectively, so as to convey control signals in a reverse direction relative to the energy transfer, basically using the same transmission technique. The control signals may be modulated with respect to frequency, phase or amplitude. Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off. In implementing this system in the arrangement of FIG. 682, the switch 1026 is either separate and controlled by the internal control unit 102, or integrated in the internal control unit 102. It should be understood that the switch 1026 should be interpreted in its broadest embodiment. This means a transistor, MCU, MCPU, ASIC FPGA or a DA converter or any other electronic component or circuit that may switch the power on and off. To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 1027 to the external signal receiver 200c. Alternatively, the energy balance can be determined by the external control unit 200b instead depending on the implementation, as mentioned above. In that case, the control signal may carry measurement results from various sensors. The amount of energy emitted from the external energy source 200a can then be regulated by the external control unit 200b, based on the determined energy balance, e.g. in response to the received control signal. This process may be repeated intermittently at certain intervals during ongoing energy transfer, or may be executed on a more or less continuous basis during the energy transfer. The amount of transferred energy can generally be regulated by adjusting various transmission parameters in the external energy source 200a, such as voltage, current, amplitude, wave frequency and pulse characteristics. This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized. This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor. With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 1026 is interconnected between the external energy source 200a and an operation device, such as an electric motor 1007 operating the apparatus 10. An external control unit 200b controls the operation of the external switch 1026 to effect proper operation of the apparatus 10. FIG. 684 illustrates different embodiments for how received energy can be supplied to and used by the apparatus 10. Similar to the example of FIG. 682, an internal energy receiver 1002 receives wireless energy E from an external energy source 200a which is controlled by a transmission control unit 200b. The internal energy receiver 1002 may comprise a constant voltage circuit, indicated as a dashed box “constant V” in the figure, for supplying energy at constant voltage to the apparatus 10. The internal energy receiver 1002 may further comprise a constant current circuit, indicated as a dashed box “constant C” in the figure, for supplying energy at constant current to the apparatus 10.

The apparatus 10 comprises an energy consuming part 10a, which may be a motor, pump, restriction device, or any other medical appliance that requires energy for its electrical operation. The apparatus 10 may further comprise an energy storage device 10b for storing energy supplied from the internal energy receiver 1002. Thus, the supplied energy may be directly consumed by the energy consuming part 10a, or stored by the energy storage device 10b, or the supplied energy may be partly consumed and partly stored. The apparatus 10 may further comprise an energy stabilizing unit 10c for stabilizing the energy supplied from the internal energy receiver 1002. Thus, the energy may be supplied in a fluctuating manner such that it may be necessary to stabilize the energy before consumed or stored. The energy supplied from the internal energy receiver 1002 may further be accumulated and/or stabilized by a separate energy stabilizing unit 1028 located outside the apparatus 10, before being consumed and/or stored by the apparatus 10. Alternatively, the energy stabilizing unit 1028 may be integrated in the internal energy receiver 1002. In either case, the energy stabilizing unit 1028 may comprise a constant voltage circuit and/or a constant current circuit. It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope of the present invention. FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window. The schematic FIG. 685 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 668; the transmitting parts of the system are excluded. The implementation of the general concept of energy balance and the way the information is transmitted to the external energy transmitter can of course be implemented in numerous different ways. The schematic FIG. 685 and the above described method of evaluating and transmitting the information should only be regarded as examples of how to implement the control system.

### Circuit Details

In FIG. 685 the symbols Y1, Y2, Y3 and so on symbolize test points within the circuit. The components in the diagram and their respective values are values that work in this particular implementation which of course is only one of an infinite number of possible design solutions.

Energy to power the circuit is received by the energy receiving coil L1. Energy to implanted components is transmitted in this particular case at a frequency of 25 kHz. The energy balance output signal is present at test point Y1. Those skilled in the art will realize that the above various embodiments of the system could be combined in many different ways. For example, the electric switch 1006 of FIG. 668 could be incorporated in any of the embodiments of FIGS. 671-677, the hydraulic valve shifting device 1014 of FIG. 671 could be incorporated in the embodiment of FIG. 670, and the gear box 1024 could be incorporated in the embodiment of FIG. 669. Please observe that the switch simply could mean any electronic circuit or component. The embodiments described in connection with FIGS. 682, 684 and 685 identify a method and a system for controlling transmission of wireless energy to implanted energy consuming components of an electrically operable apparatus. Such a method and system will be defined in general terms in the following. A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance. The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference. When controlling the energy transmission, the amount of transmitted wireless energy may be decreased if the detected energy balance change implies that the energy balance is increasing, or vice versa. The decrease/increase of energy transmission may further correspond to a detected change rate. The amount of transmitted wireless energy may further be decreased if the detected energy difference implies that the received energy is greater than the used energy, or vice versa. The decrease/increase of energy transmission may then correspond to the magnitude of the detected energy difference. As mentioned above, the energy used for the medical device may be consumed to operate the medical device, and/or stored in at least one energy storage device of the medical device. When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance. When the derivative is determined over time of a measured electrical parameter related to the amount of consumed and/or stored energy, the derivative may be determined for a monitored voltage and/or current related to the energy balance. The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals. In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses. A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied. When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency. The circuit formed by the first electric circuit and the external energy source may have a first characteristic time period or first time constant, and when effectively varying the transmitted energy, such frequency time period may be in the range of the first characteristic time period or time constant or shorter. A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device. In one embodiment at least one battery may be a part of or replace the energy-transforming device 1002 to supply energy to the apparatus 10 over a power supply line. In one embodiment the battery is not rechargeable. In an alternative embodiment the battery is rechargeable. The battery supply may of course be placed both remote to and incorporated in the device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

FIGS. 686-689 show in more detail block diagrams of four different ways of hydraulically or pneumatically powering an implanted apparatus according to the invention.

FIG. 686 shows a system as described above with. The system comprises an implanted apparatus and further a separate regulation reservoir 1013, a one way pump 1009 and an alternate valve 1014.

FIG. 687 shows the apparatus 10 and the regulation reservoir 1013. By moving the wall of the regulation reservoir or changing the size of the same in any other different way, the adjustment of the apparatus may be performed without any valve, just free passage of fluid any time by moving the reservoir wall.

FIG. 688 shows the apparatus 10, a two way pump 1009 and the regulation reservoir 1013.

FIG. 689 shows a block diagram of a reversed servo system with a first closed system controlling a second closed system. The servo system comprises a regulation reservoir 1013 and a servo reservoir 1050. The servo reservoir 1050 mechanically controls an implanted apparatus 10 via a mechanical interconnection 1054. The apparatus has an expandable/contactable cavity. This cavity is preferably expanded or contracted by supplying hydraulic fluid from the larger adjustable reservoir 1052 in fluid connection with the apparatus 10. Alternatively, the cavity contains compressible gas, which can be compressed and expanded under the control of the servo reservoir 1050.

The servo reservoir 1050 can also be part of the apparatus itself.

In one embodiment, the regulation reservoir is placed subcutaneous under the patient's skin and is operated by pushing the outer surface thereof by means of a finger. This system is illustrated in FIGS. 58a-c. In FIG. 690a, a flexible subcutaneous regulation reservoir 1013 is shown connected to a bulge shaped servo reservoir 1050 by means of a conduit 1011. This bellow shaped servo reservoir 1050 is comprised in a flexible apparatus 10. In the state shown in FIG. 690a, the servo reservoir 1050 contains a minimum of fluid and most fluid is found in the regulation reservoir 1013. Due to the mechanical interconnection between the servo reservoir 1050 and the apparatus 10, the outer shape of the apparatus is contracted, i.e., it occupies less than its maximum volume. This maximum volume is shown with dashed lines in the figure.

FIG. 690b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 10 so that it occupies its maximum volume, thereby stretching the stomach wall (not shown), which it contacts. The regulation reservoir 1013 is preferably provided with means 1013a for keeping its shape after compression. This means, which is schematically shown in the figure, will thus keep the apparatus in a stretched position also when the user releases the regulation reservoir. In this way, the regulation reservoir essentially operates as an on/off switch for the system. An alternative embodiment of hydraulic or pneumatic operation will now be described with reference to FIGS. 691 and 692a-c. The block diagram shown in FIG. 691 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 10 having an expandable/contactable cavity is in turn controlled by the larger adjustable reservoir 1052 by supply of hydraulic fluid from the larger adjustable reservoir 1052 in fluid connection with the apparatus 10. An example of this embodiment will now be described with reference to FIG. 692a-c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 692a, the servo reservoir 1050 contains a minimum of fluid and most fluid is found in the regulation reservoir 1013. The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 10. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 10. In other words, in this reversed servo, a small volume in the regulation reservoir is compressed with a higher force and this creates a movement of a larger total area with less force per area unit.

Like in the previous embodiment described above with reference to FIGS. 690a-c, the regulation reservoir 1013 is preferably provided with means 1013a for keeping its shape after compression. This means, which is schematically shown in the figure, will thus keep the apparatus 10 in a stretched position also when the user releases the regulation reservoir. In this way, the regulation reservoir essentially operates as an on/off switch for the system.

Although the different parts described above have specific placements on the drawings it should be understood that these placements might vary, depending on the application.

The lubricating fluid used in any of the embodiments herein is preferably a biocompatible lubricating fluid imitating the synovial fluid of the natural hip joint. According to one embodiment the lubricating fluid is Hyaluronic acid.

In all of the embodiments above it is conceivable that the conduit is excluded and that the channel or channels are in direct connection with the reservoir or the injection port. Please note that any embodiment or part of embodiment as well as any method or part of method could be combined in any way. All examples herein should be seen as part of the general description and therefore possible to combine in any way in general terms. Please note that the description in general should be seen as describing both of an apparatus and a method.

The various aforementioned features may be combined in any way if such combination is not clearly contradictory. The invention will now be described in more detail in respect of preferred embodiments and in reference to the accompanying drawings. Again, individual features of the various embodiments may be combined or exchanged unless such combination or exchange is clearly contradictory to the overall function of the device.

### S: Artificial Heart Valve

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating an artificial heart valve, examples of such an artificial heart valves will now be described.

The invention will be described in the following with reference to a human blood vessel, and may also be described below as being placed in a human heart. It should, however, be pointed out that these are merely examples given in order to facilitate the reader's understanding of the invention; the artificial valve 100 can be used at more or less any point in the circulatory system of any mammal.

In addition, the artificial valve can be used in order to replace a biological valve, as an alternative to which it can be used in order to supplement a biological valve, or to create a “valve function” in a position where the body is normally not equipped with a valve function.

As has been mentioned above, the present invention discloses an artificial valve for implantation in a mammal body, in or adjacent to a mammal blood vessel. The valve comprises a casing and a closing mechanism, and at least part of the closing mechanism is a first moving part which is adapted to make movements relative to the casing. These movements enable the first moving part of the closing mechanism to assume an open and a closed position for opening and closing, respectively, the blood flow through the blood vessel, as well as to positions in between said open and closed positions. As also mentioned previously, the first moving part of the closing mechanism is adapted to initiate and carry out its opening movement as the result of a predefined threshold value being reached by a physical parameter of the mammal or a functional parameter of a device used by the mammal. One of the parameters which can be used in order to initiate the opening movement of the first moving part in one embodiment is the difference in blood pressure between an inner and an outer side of the valve in its closed position. Before a description is given of other parameters which can be used, or of an example of the mechanism which triggers the opening at a certain threshold, an advantage given by the invention will be described in the following: In a mammal, for example a human being, in the “normal” function of the mammal's heart, the blood in the mammal's heart flows from the heart through a natural or known artificial valve, which opens due to the increased blood pressure in the heart's systolic phase. However, if we look at a mammal with reduced circulation and oxygen supply in the coronary arteries, the valve may be used to be implanted in the aorta between the exit to the coronary arteries and the exit to the carotid arteries, as opposed to a natural valve and known artificial valves, which are placed before the coronary arteries. Since the valve of the present invention is opened by a parameter which reaches a predefined threshold, the valve can be made to remain closed slightly longer than a valve with the “normal” function, i.e. a valve which opens more or less instantly as the blood pressure mounts. Since the inventive valve can be made to remain closed slightly longer than a “normal” valve, the blood pressure on the “heart side” of the valve will build up to a level which is higher than the blood pressure which causes a normal valve to open, which in turn will cause an increased amount of blood to flow into the coronary arteries, which will then serve to alleviate the circulatory problems of the mammal. A valve 100 in a certain embodiment which will be described in more detail later is shown in fig 621 in a human heart in the position, i.e. between the exit to the coronary arteries and the exit to the carotid arteries. The difference between the inventive valve as compared to a natural valve is thus in this embodiment that the inventive valve opens at a slightly higher pressure than the normal valve. A suitable threshold pressure for initiating an opening movement of the valve has been found to be 10 mm Hg, although the range of 10-30 mm Hg has also been found to be useful. The term “blood pressure” is here used in the sense of a difference in pressure between two sides of the valve in the valve's closed position, i.e. the inner and outer side of the valve.

Other parameters which are also used in a valve to initiate an opening movement, alone or in combinations with each other, are:
the blood flow at a defined point in the circulatory system of the mammal,
a physical parameter which is related to the contraction of a muscle at a defined point in the mammal,
a body generated parameter related to the contraction of the mammal's heart muscle,
a device generated signal related to the contraction of the mammal's heart muscle.

Before the “triggering” of the opening movement is described in more detail, some embodiments of the closing mechanism as such and its moving part or parts will first be described.

FIG. 622a shows a side view of a first embodiment of an artificial valve 10 of the invention. As seen in FIG. 622a, in this embodiment, the closing mechanism of the valve comprises a first moving part 11, suitably essentially shaped as a disc in order to enable the closing of a blood vessel. The valve 10 also comprises a casing 12 in which the moving part 11 is housed.

As is also shown in FIG. 622a, the casing 12 comprises a ring, which is shaped so that the disc 11 may rotate in the casing to assume open and closed positions, as well as positions in between said open and closed positions. In order to enable the rotation of the disc 11, the valve 10 also comprises first 13 and second 13′ hinges arranged in the casing, about which hinges the disc can rotate. As will be realized, in the open position, shown in FIG. 622b, the disc 11 is essentially perpendicular to the casing 12, while it in the closed position is essentially aligned with an inner wall of the casing 12.

FIG. 622b shows a side view of the embodiment 10 in the open position.

In a second embodiment 20, shown in a side view in FIG. 623a, the closing mechanism of the valve comprises a first 21 and a second 22 moving part, each of which moving part is movably hinged about respective first 23, 23′ and second 24, 24′, hinges in a ring-shaped casing 12. The first 21 and second 22 parts can move about their respective hinges to assume a closed and an open position, as well as positions in between said open and closed positions in order to close or limit the blood flow through said blood vessel.

FIG. 623b shows the valve 20 in a side view. As can be seen here, the two parts 21 and 22 are essentially flat halves of a flat disc, while FIGS. 623c and 623d show an embodiment in which the two moving parts 21, 22, come together to form a cupola in the closed position of the valve 20.

In a further embodiment 30 of the inventive valve, which is shown in a plan view in FIG. 624a, the closing mechanism of the valve comprises first 31, second 32 and third 33 moving parts, each of which is movably hinged about respective first 34, 35, 36 and second 34′, 35′, 36′, hinges in a ring-shaped casing 37.

In this embodiment, the first 31, second 32 and third 33 moving parts can move about their respective hinges to assume a closed and an open position as well as positions in between said open and closed positions in order to close or limit the blood flow through said blood vessel,

The words “open” and “closed” positions for the embodiments 20 and 30 of the valve should here be taken to mean that each moving part can assume a closed and an open position, but that each part needs to be in its closed position in order for the blood flow through a blood vessel to be closed, and that a maximally open valve is achieved when each moving part is in its open position.

As shown in side views in FIGS. 624b and 624c, in similarity to the embodiment 20 shown in FIGS. 623c and 623d, in the “three part embodiment” 30, the moving parts suitably come together to form a cupola in the closed position.

FIG. 624d shows the embodiment 30 in the open position, and FIG. 624e shows one of the cupola parts 31 with its hinges 34, 34′. As shown in the views of FIGS. 623a and 624a, in the embodiments 20 and 30, the first and second hinges of at least one of the moving parts of the valve of those embodiments are positioned at or adjacent to a meeting point of the moving parts. Also suitably, which can also be seen in the views of FIGS. 623a and 624a, the first and second hinges of at least one of the moving parts of the valve of those embodiments are placed at substantially opposite distal ends of the moving part along the casing. This positioning of the hinges allows for a smoother and easier movement of the moving parts of the valve, as opposed to the hinges of traditional valves, which are usually placed at a center position of the moving part. FIGS. 625a and 625b show the valve 30 in a blood vessel, in the open (FIG. 625a) and closed (fig 625b) positions. It can be gleaned from these drawings that in some embodiments, the flow of blood will be used to assist in the closing movement of the moving parts, as well as possibly (FIG. 625a) also in the opening movement of the moving parts. In the embodiments shown in FIGS. 621-S5 and described above, the mechanism for letting the valve initiate its opening movement can be powered, i.e. the valve comprises means for actively (i.e. using a power supply which is at least in part external to the blood vessel) initiating and carrying out the opening movement or movements. As an alternative, at least in the embodiments in which the opening movement is triggered by means of a certain threshold value of the blood pressure or blood flow, this can be achieved by passive means, i.e. without any supply of external energy. An example of passive such means will be described below, with reference to FIGS. 637a and 637b. As shown in FIGS. 637a and 637b, one of the moving parts, generically shown as A, comprises a protruding edge F which creates a “step” G on the side of the protruding part which is closest to the casing E. The casing, in turn, comprises a movable protruding part B, which is lodged in a groove D in the casing, and is attached to the casing by means of a spring C. Thus, when the moving part A performs its opening movement, shown by the arrow in FIG. 637a, it will be delayed in that movement by the contact between the edge G and the movable part/spring mechanism of the casing. Once the step/edge G has cleared the movable part B, however, the braking effect will cease. When the moving part A is to carry out an opening movement, FIG. 637b, the outside of the step F will come into contact with the moving part, and will not be “caught” by the movable part B to the same extent as in the opening movement. As can be understood, the opening resistance caused by the mechanism of FIGS. 637a and 637b, can be dimensioned to correspond to a certain level of blood pressure or blood flow. Some different way of how the closing mechanism of the embodiments can be powered in its movements will now be described, before a different version of the valve is described. The powering of the movements of the closing mechanism is shown in FIGS. 638-640 below, and is based on the casing having a first H and second H′ casing part, with the first part being displaceable in relation to the second part in order to cause the opening and/or closing movement. Suitably, the first H and second H′ casing parts each comprise a ring, with the two rings being concentric to each other, and with one of the first or second rings being adapted to move in relation to the other part in order to cause the closing and/or opening movement. As can be seen in FIGS. 638a and 638b, the two parts H′ and H of the casing each constitute rings and can be made to move away from each other or towards each other, i.e. essentially in the direction of the “axis” of a blood vessel. Suitably, only one of the casing parts should be made to move. In the embodiment of FIGS. 638a and 18b, the ring H, which is the most distant from the moving parts of the cupola, has one end of each of three mechanical elements K, for example three pins, attached to it, with the other ends of the pins being attached to one each of the moving parts of the cupola. As the distance between the two parts of the casing, H and H′, is made to increase or decrease, by means of, for example, magnets and coils, the pins will cause the cupola parts to move about their hinges and open, FIG. 640b, or close, FIG. 638a. FIGS. 639a-639d show another embodiment in which the casing parts are also concentric rings H′, H. However, in this embodiment, the opening and/or closing movement of the cupola parts is obtained by letting the rings rotate in relation to each other, suitably with only one of the rings rotating. As can be seen in FIGS. 639a-639d, the ring H which can be made to rotate, for example by means of interaction between springs on one ring and coils on the other, comprises three pins, J, which can move in corresponding openings I of the other ring H′. As can also be seen in FIGS. 639a-639d, the cupola parts comprise a groove N (not a through-going groove though) in which the pin J can run. The groove N is slanted in the cupola part, so that rotation of the ring H with the pins J will cause the cupola parts to open or close, depending on the direction of rotation of the ring H.

FIG. 640 shows another embodiment of how the cupola parts may be made to open and/or close actively as well as passively; in this embodiment as well, the casing comprises an upper N and a lower O ring shaped part, which are essentially concentric. One of the ring shaped parts, O, comprises a groove P, which consists of vertical and slanted parts, in an alternating fashion. A pin M from each cupola part runs in this groove. If the blood pressure increases, the cupola part will open, since the pin will move in a vertical (i.e. essentially parallel with the extension of a blood vessel) part of the groove, and can also be closed when the blood begins to flow in the reverse direction, i.e. during the diastolic phase of the heart. However, if the ring O with the groove P in it is made to rotate, the pin will be forced to move in or by a slanted part of the groove, which will also cause the cupola part to perform a closing or opening movement, depending on the direction of rotation of the ring. A mechanism for making the ring O rotate is indicated as Q in FIG. 638. In an alternative embodiment 50 of the valve, shown in a plan view in FIG. 626a, together with a blood vessel 52, the closing mechanism of the valve comprises an elongated and essentially flat plate 51 which is adapted to, when the valve 50 is arranged in or adjacent to an opening in the blood vessel 52, move into this opening in a direction which is essentially perpendicular to the blood vessel in order to limit or close the blood flow through said vessel. The direction of movement of the plate 51 is indicated by means of an arrow “A” in FIG. 626a. The closing mechanism of the valve is adapted to be powered in its movements to the closed position in part or entirely by means of a power source external to said blood vessel. As can be seen in FIG. 626a, in one embodiment, the flat plate 51 is given a curved or semicircular shape at the end 53 of the plate 51 which will be the first to enter an opening in the blood vessel 52 during a closing movement, and by means of the curved shape of the end 53, the plate 51 is then adapted to fit against a distal inner wall of the blood vessel 52 in order to close or limit the passage of blood in said blood vessel. The blood vessel 52 is shown in a perspective view in FIG. 626b, together with an opening 54 which is made in the blood vessel in order to admit the plate 51. The flat plate 51 is arranged in or adjacent to a casing 61, which is shown in a perspective view in FIG. 627. As can be seen in FIG. 627, in one embodiment, an outer wall 62 of the casing 61 is concavely curved so that it will essentially coincide with the outer shape of a blood vessel against which the casing 61 will be arranged. The curved outer wall 62 also comprises an opening 63 for the plate 51, through which opening the plate can move in its movements. In this embodiment, the tolerance between the dimensions of the opening and the plate should be such that the movements of the plate 51 are enabled, but also such that leakage of blood between the plate 51 and the opening 63 is essentially eliminated. In one embodiment, also shown in FIG. 627, in order to make it possible to attach the valve 50 securely to a blood vessel, the casing 61 also comprises at least a first curved protruding part 64 for surrounding at least part of the circumference of a blood vessel. In another embodiment, the casing 61 also comprises a second curved protruding part 65 for surrounding at least part of the circumference of a blood vessel, so that the two parts 64, 65 may be arranged on opposite sides of a blood vessel to which the valve 50 is to be attached. In some patients or in some positions in a patient's body, it may be possible to attach the valve 50 to a blood vessel 52 by means of letting the casing 61 of the valve surround the blood vessel entirely, which is shown in FIG. 628. For such applications, the valve can be made to also comprise a detachable part 71 for attachment to the casing 61 or to one or more of the protruding parts 64, 65. The valve may then be made to completely surround a blood vessel by means of at least one protruding part and said detachable part and/or by means of a curved outer wall of the valve, as shown in FIG. 628. In the embodiments with the flat plate 51, the plate will thus in its closing movements move into (and out from, in an opening movement) a position in a blood vessel. In one embodiment, show in a side view in FIGS. 628a and 628b, in order to guide the plate 51 in these movements, the casing 61 of the valve also comprises a protruding guide 81 for guiding the movements of the plate 51 in the blood vessel 52. The guide 81 is thus intended for being arranged inside the blood vessel 81, and is for that reason essentially shaped to coincide with the outer form of the plate, with a certain tolerance to enable the plate to move in the guide. The guide 81 can be seen as an outer rail for the plate 51, and can comprise grooves for the plate 51 to move in.

FIG. 629a also shows a vascular graft 82, by means of which the valve may be attached to the blood vessel 52.

FIG. 630 shows a cross sectional view of a blood vessel 52, adjacent to which a valve 50 of the “flat plate” embodiment has been arranged, with protruding parts 64, 65, to which the detachable part 71 has been attached, so that the casing entirely surrounds the blood vessel 52. The flat plate 51 is also shown in FIG. 630, with its direction of movement being indicated by the arrow “A”. In some embodiments, the valve 50 will also preferably comprising a biasing mechanism for biasing the plate to an open position, so that the powered movement has to overcome a biasing force in order to perform the closing movement of the plate 51. Suitably, such a biasing mechanism comprises a spring mechanism. This is shown in FIG. 631, which shows an open side view of the valve 50 arranged adjacent to a blood vessel 52, and shows a possible spring mechanism 82 arranged in the casing 61. As can be seen in FIG. 631, the spring mechanism cooperates with an abutment on the plate 51, in order to bias the plate 51 to an open position in the casing 61. Turning now to how and when the closing movements of the valve will be made to take place, this will be described in the following, and will be shown using the drawings of the cupola embodiment as an example. It should however be pointed out that the same principle may be used in other embodiments of the invention, such as for example, the “flat plate” embodiment 50. As shown in FIGS. 624a and 624c, the valve may be made to also comprise a receiving device, shown as 102 in FIGS. 624a and 624c. Although the receiving device is shown in three parts in FIGS. 624a and 624c, the receiving device can naturally also comprise one or two parts, or more than three parts. The receiving device or devices serve to receive a closing signal and for supplying this closing signal to the closing mechanism, which in turn is adapted to close upon the reception of the closing signal. The closing mechanism and the receiving device can be integrated into one unit, as shown throughout in the drawings, or they may be two separate units in the valve. The exact design of the receiving device 102 can vary, but in a preferred embodiment, the receiving device is adapted to receive the opening and/or closing signal as an electrical signal. This is shown in FIGS. 632a and 632b, which also show, see FIG. 632a, that the signal may be received via cabling, which is connected to the receiving device, or, FIG. 632b, that the signal may be received wirelessly, i.e. as radio signals, so that the receiving device or devices comprise a wireless receiver. In the case of a wireless signal, the receiving device may in some embodiments also comprise a demodulator for demodulating a received wireless signal. Turning now to more details of how the moving parts of the closing mechanism of the various embodiments are made to perform their closing movements, this can be achieved in a large number of ways within the scope of the present invention, as will be obvious to those skilled in the field. However, as shown in FIG. 633, in one embodiment, the closing mechanism may comprise one or more magnets, each of which interacts with a coil 83 in order to create movement of the moving parts 31, 32, and 33. As indicated in FIG. 632, each of the coils is arranged on the casing 37 at a central position for each moving part 31, 32, 33, with each of the interacting magnets being arranged at a position on a moving part which is immediately adjacent to the position of a coil. In the plate embodiment 50, the magnet is instead preferably placed on the plate, and the coil is housed inside the casing. The coil 83 is also shown in the plate embodiment in FIG. 631. In the “spring and coil” embodiment of the closing mechanism, the motion of the moving parts is caused by passing an AC current through the coils. In another embodiment, the closing mechanism comprises a mechanical element which is involved in the closing movements. A suitable example of such a mechanical element is a rotatable shaft, which may, for example, in the case of the “cupola embodiment” 20, 30, be arranged to interact with the hinges of the moving parts to cause the moving parts to open and/or to close. In the “plate embodiment” 50, the rotatable shaft will instead be arranged inside the housing, and, for example, interacts with the plate by means of cogs. Suitably, if a shaft is used, the rotatable shaft is attached to an engine which rotates the shaft, with the rotation of the shaft being controlled by the signals received by the receiving device. An example of the shaft embodiment is shown in FIG. 634, in which a symbolic shaft 76 is shown next to each of the hinge positions of the moving parts of the cupola. The engine which drives the shafts is not shown in FIG. 634. Turning now to the signals which the receiving device of the valve is adapted to receive, these signals will in some embodiments be received from a source such as a sensor or some other device which is external to the valve, with said source however being connected to the receiving device, for example by means of cabling or wirelessly, as described above. Such a sensor is shown in FIGS. 632a and 12b with the reference number 8. The signals which the receiving device is adapted to receive from this external source may be based upon a variety of parameters, some examples of which will be given below. It should be understood that these signals may also be combined, so that the receiving device receives input from more than one source or from more than one measurement: In one embodiment, the receiving device of the valve is adapted to receive input signals which are the result of the blood pressure or blood flow at a defined point in the circulatory system of the user of the valve reaching a predetermined threshold, which thus indicates that a closing movement should be carried out by the valve. In one embodiment, the receiving device of the valve is adapted to receive inputs signals as the result of a parameter which is related to the contraction of a muscle at a defined point in the user of the valve reaching a predetermined threshold. For example, this may be a measurement of the heart's phases, so that the valve is made to close at predefined points of the systolic and/or diastolic phases of the heart. In general, with regard to the valve operating in conjunction with the heart in a predefined manner, the input signals to the receiving device may be received as the result of one or more predefined body generated parameters which is/are related to the contraction of the heart muscle reaching a predetermined threshold. Examples of such parameters are those mentioned, such as blood pressure, heart contractions (for example movement or bending or volume) and heart anti-contractions, and also heart electrical body generated signals. In one embodiment, the artificial valve is adapted to cooperate with another device used by the mammal in question. Thus, in such an embodiment, the receiving device is adapted to receive the input signals as the result of a device generated signal, suitably related to the contraction of the heart. An example of such a device may be a so called pacemaker, and in this case, the input signals would be signals which indicate that the mammal's heart has reached a certain phase at which the artificial valve should close. The pacemaker will then serve the role of the device 200 of FIGS. 632a and 632b. Thus, the receiving device can be adapted to receive said signal as the result of a certain threshold value being reached by a physical parameter of the mammal or a functional parameter of a device,

As described above, the valve may be designed to cooperate with an external device such as a sensor or a device used by the user, such as a pacemaker. However, in alternative embodiments, as a complement or replacement to external sensors and devices, the valve will in itself comprise a sensor for sensing one or more parameters, such as a physical parameter of the mammal or a functional parameter of another device, such as, for example, the parameters enumerated above; such a sensor will then also generate input signals to the receiving device of the valve. This embodiment is shown in FIG. 635, in which the sensor 79 is shown as being arranged on the casing of the valve. In one embodiment, the valve in addition comprises a control device for controlling the opening and closing of the valve, i.e. the movement of the moving parts of the valve. In this embodiment, the control device receives the input signals instead of or via the receiving device, processes the signals, and controls the operation of the valve accordingly. As indicated in FIG. 636, such a control device 87 suitably comprises a processor of some kind, for example a microprocessor, as well as a memory for storing executable code, and possibly also for storing parameters which are relevant to the operation of the valve, e.g. threshold parameters such as those mentioned above, and others. Suitably, the control device controls the operating mechanism using input parameters which are received via the receiving device and/or sensed by an internal sensor. As mentioned previously, the operating mechanism of the valve will in one embodiment comprise at least one magnet and at least one coil which interact with each other in order to cause an opening and/or closing movement of at least one of the moving parts of the valve. In an alternative embodiment, as a complement or alternative to the spring/coil mechanism, the operating mechanism is attached to the casing, in the “cupola embodiments”, or housed in the casing, in the case of the “plate embodiment”, and comprises at least two parts, with a first part being adapted to move in relation to a second part to cause an opening or closing movement of said moving parts. Suitably, the first part is then the rotating shaft mentioned previously, which in the case of the “cupola embodiments” is adapted to rotate perpendicularly along the periphery of the blood vessel in which the valve may be implanted. Regarding the choice of material for the parts of the valve, the moving parts are suitably made of titanium, but any suitable material could be used; the casing may preferably be manufactured in a ceramic material, but for example stainless steel or plastic materials can also be used. The hinges may be manufactured in titanium, stainless steel, plastic material or ceramics or any combination thereof. In one embodiment, the moving parts of the valve are at least partially given a structured surface, i.e. a surface which has a pattern or a texture on it, since this has been found to facilitate the growth of mammal material upon a surface. In one embodiment, the moving parts of the valve are at least partially covered by mammal valve material, such as that taken from a cow, a pig or a human being. As shown in the drawings, the moving parts of the cupola, which can be two or more, are all essentially equally shaped, so that they represent essentially equal parts of the cupola. This is one embodiment, but embodiments in which the cupola is formed by unequally shaped parts are also within the scope of the present invention, as well as embodiments which use more than three moving parts to form a cupola.

The invention also discloses methods for implanting a valve into a mammal patient.

According to one embodiment of such a method, the following steps are carried out:
inserting a needle or a tube-like instrument into the patient's thoraxial or abdominal or pelvic cavity,
using the needle or tube-like instrument to fill a part of the patient's body with gas, thereby expanding said cavity,
placing at least two laparoscopic trocars in said cavity,
inserting a camera through one of the laparoscopic trocars into said cavity,
inserting at least one dissecting tool through one of said at least two laparoscopic trocars,
dissecting an area of a potential place for a valve of a blood vessel,
placing a device in said blood vessel, and
suturing in steps.

In another embodiment, the method comprises the steps of:
cutting the skin of the mammal patient,
dissecting an area of a blood vessel,
placing a device in said blood vessel, and
suturing in steps.

In another embodiment, the method comprises the steps of:
inserting a needle or a tube-like instrument into the patient's thoraxial cavity,
using the needle or tube-like instrument to fill a part of the patient's body with gas, thereby expanding said thoraxial cavity,
placing at least two laparoscopic trocars in said cavity,
inserting a camera through one of the laparoscopic trocars into said cavity,
inserting at least one dissecting tool through one of said at least two laparoscopic trocars,
dissecting an area of a heart valve,
placing a device in the patient's heart or a connecting blood vessel, and
suturing in steps.

In another embodiment, the method comprises the steps of:
cutting the skin in the thoraxial wall of a mammal patient,
dissecting an area of the artificial heart valve,
placing a device in the patient's heart or in a connecting blood vessel, and
suturing in steps.

Suitably, but not necessarily, the dissection of the methods mentioned above includes the following steps:
dissecting a path for a cable into the right atrium of the heart
cutting the skin and dissecting a subcutaneous place for a control unit, similar to a pacemaker position
introducing the cable backwards from the right atrium of the heart to the position of the control unit following the venous blood vessels.

In this embodiment, the cable is suitably made to reach vein subclavia or vein cephalica and to exit from that vessel. Also, suitably, the placing of the inventive valve includes placing a control unit in the subcutaneous area and connecting to a cable for supplying the closing and/or opening signal to the valve.

The method also, in one embodiment, includes providing a power supply to wirelessly supply energy to the valve of the invention, wherein the dissection and placing includes the following steps:
dissecting the area outside said heart valve
placing a wireless control unit including a power supply to wirelessly supply the closing signal to said heart valve

The invention also discloses a system for powering and controlling an artificial device or apparatus such as that disclosed by the invention.

FIG. 666 illustrates a system for treating a disease comprising an apparatus 10 of the present invention placed in the abdomen of a patient. An implanted energy-transforming device 302 is adapted to supply energy consuming components of the apparatus with energy via a power supply line 303. An external energy-transmission device 200 for non-invasively energizing the apparatus 10 transmits energy by at least one wireless energy signal. The implanted energy-transforming device 302 transforms energy from the wireless energy signal into electric energy which is supplied via the power supply line 303.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field.

The wireless energy-transmission device 200 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal.

Generally speaking, the energy-transforming device 302 is provided for transforming wireless energy of a first form transmitted by the energy-transmission device 200 into energy of a second form, which typically is different from the energy of the first form. The implanted apparatus 100 is operable in response to the energy of the second form. The energy-transforming device 302 may directly power the apparatus with the second form energy, as the energy-transforming device 302 transforms the first form energy transmitted by the energy-transmission device 200 into the second form energy. The system may further include an implantable accumulator, wherein the second form energy is used at least partly to charge the accumulator.

Alternatively, the wireless energy transmitted by the energy-transmission device 200 may be used to directly power the apparatus, as the wireless energy is being transmitted by the energy-transmission device 200. Where the system comprises an operation device for operating the apparatus, as will be described below, the wireless energy transmitted by the energy-transmission device 200 may be used to directly power the operation device to create kinetic energy for the operation of the apparatus.

The wireless energy of the first form may comprise sound waves and the energy-transforming device 302 may include a piezo-electric element for transforming the sound waves into electric energy. The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus.

Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal.

The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released non-magnetic wireless energy is used for operating the apparatus.

The external energy-transmission device 200 also includes a wireless remote control having an external signal transmitter for transmitting a wireless control signal for non-invasively controlling the apparatus. The control signal is received by an implanted signal receiver which may be incorporated in the implanted energy-transforming device 302 or be separate there from. The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field. The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals.

FIG. 667 illustrates the system of FIG. 666 in the form of a more generalized block diagram showing the apparatus 10, the energy-transforming device 302 powering the apparatus 10 via power supply line 303, and the external energy-transmission device 200, The patient's skin SK, generally shown by a vertical line, separates the interior of the patient to the right of the line from the exterior to the left of the line.

FIG. 668 shows an embodiment identical to that of FIG. 667, except that a reversing device in the form of an electric switch 306 operable for example by polarized energy also is implanted in the patient for reversing the apparatus 10. When the switch is operated by polarized energy the wireless remote control of the external energy-transmission device 200 transmits a wireless signal that carries polarized energy and the implanted energy-transforming device 302 transforms the wireless polarized energy into a polarized current for operating the electric switch 306. When the polarity of the current is shifted by the implanted energy-transforming device 302 the electric switch 306 reverses the function performed by the apparatus 10.

FIG. 669 shows an embodiment identical to that of FIG. 667, except that an operation device 307 implanted in the patient for operating the apparatus 10 is provided between the implanted energy-transforming device 302 and the apparatus 10. This operation device can be in the form of a motor 307, such as an electric servomotor. The motor 307 is powered with energy from the implanted energy-transforming device 302, as the remote control of the external energy-transmission device 200 transmits a wireless signal to the receiver of the implanted energy-transforming device 302.

FIG. 670 shows an embodiment identical to that of FIG. 667, except that it also comprises an operation device is in the form of an assembly 308 including a motor/pump unit 309 and a fluid reservoir 310 is implanted in the patient. In this case the apparatus 10 is hydraulically operated, i.e. hydraulic fluid is pumped by the motor/pump unit 309 from the fluid reservoir 310 through a conduit 311 to the apparatus 10 to operate the apparatus, and hydraulic fluid is pumped by the motor/pump unit 309 back from the apparatus 10 to the fluid reservoir 310 to return the apparatus to a starting position. The implanted energy-transforming device 302 transforms wireless energy into a current, for example a polarized current, for powering the motor/pump unit 309 via an electric power supply line 312. Instead of a hydraulically operated apparatus 10, it is also envisaged that the operation device comprises a pneumatic operation device. In this case, the hydraulic fluid can be pressurized air to be used for regulation and the fluid reservoir is replaced by an air chamber. In all of these embodiments the energy-transforming device 1002 may include a rechargeable accumulator like a battery or a capacitor to be charged by the wireless energy and supplies energy for any energy consuming part of the system. As an alternative, the wireless remote control described above may be replaced by manual control of any implanted part to make contact with by the patient's hand most likely indirect, for example a press button placed under the skin.

FIG. 671 shows an embodiment comprising the external energy-transmission device 200 with its wireless remote control, the apparatus 10, in this case hydraulically operated, and the implanted energy-transforming device 302, and further comprising a hydraulic fluid reservoir 313, a motor/pump unit 309 and an reversing device in the form of a hydraulic valve shifting device 314, all implanted in the patient. Of course the hydraulic operation could easily be performed by just changing the pumping direction and the hydraulic valve may therefore be omitted. The remote control may be a device separated from the external energy-transmission device or included in the same. The motor of the motor/pump unit 309 is an electric motor. In response to a control signal from the wireless remote control of the external energy-transmission device 200, the implanted energy-transforming device 302 powers the motor/pump unit 309 with energy from the energy carried by the control signal, whereby the motor/pump unit 309 distributes hydraulic fluid between the hydraulic fluid reservoir 313 and the apparatus 10. The remote control of the external energy-transmission device 200 controls the hydraulic valve shifting device 314 to shift the hydraulic fluid flow direction between one direction in which the fluid is pumped by the motor/pump unit 309 from the hydraulic fluid reservoir 313 to the apparatus 10 to operate the apparatus, and another opposite direction in which the fluid is pumped by the motor/pump unit 309 back from the apparatus 10 to the hydraulic fluid reservoir 313 to return the apparatus to a starting position.

FIG. 672 shows an embodiment comprising the external energy-transmission device 200 with its wireless remote control, the apparatus 10, the implanted energy-transforming device 302, an implanted internal control unit 102 controlled by the wireless remote control of the external energy-transmission device 200, an implanted accumulator 316 and an implanted capacitor 317. The internal control unit 102 arranges storage of electric energy received from the implanted energy-transforming device 302 in the accumulator 316, which supplies energy to the apparatus 10. In response to a control signal from the wireless remote control of the external energy-transmission device 200, the internal control unit 102 either releases electric energy from the accumulator 316 and transfers the released energy via power lines 318 and 319, or directly transfers electric energy from the implanted energy-transforming device 302 via a power line 320, the capacitor 317, which stabilizes the electric current, a power line 321 and the power line 319, for the operation of the apparatus 10.

The internal control unit is preferably programmable from outside the patient's body. In a preferred embodiment, the internal control unit is programmed to regulate the apparatus 10 according to a pre-programmed time-schedule or to input from any sensor sensing any possible physical parameter of the patient or any functional parameter of the system.

In accordance with an alternative, the capacitor 317 in the embodiment of FIG. 672 may be omitted. In accordance with another alternative, the accumulator 316 in this embodiment may be omitted.

FIG. 673 shows an embodiment identical to that of FIG. 667, except that a battery 322 for supplying energy for the operation of the apparatus 10 and an electric switch 323 for switching the operation of the apparatus 10 also are implanted in the patient. The electric switch 323 may be controlled by the remote control and may also be operated by the energy supplied by the implanted energy-transforming device 302 to switch from an off mode, in which the battery 322 is not in use, to an on mode, in which the battery 322 supplies energy for the operation of the apparatus 10.

FIG. 674 shows an embodiment identical to that of FIG. 673, except that an internal control unit 102 controllable by the wireless remote control of the external energy-transmission device 200 also is implanted in the patient. In this case, the electric switch 323 is operated by the energy supplied by the implanted energy-transforming device 302 to switch from an off mode, in which the wireless remote control is prevented from controlling the internal control unit 102 and the battery is not in use, to a standby mode, in which the remote control is permitted to control the internal control unit 102 to release electric energy from the battery 322 for the operation of the apparatus 10.

FIG. 675 shows an embodiment identical to that of FIG. 674, except that an accumulator 316 is substituted for the battery 322 and the implanted components are interconnected differently. In this case, the accumulator 316 stores energy from the implanted energy-transforming device 302. In response to a control signal from the wireless remote control of the external energy-transmission device 200, the internal control unit 102 controls the electric switch 323 to switch from an off mode, in which the accumulator 316 is not in use, to an on mode, in which the accumulator 316 supplies energy for the operation of the apparatus 10. The accumulator may be combined with or replaced by a capacitor.

FIG. 676 shows an embodiment identical to that of FIG. 640, except that a battery 322 also is implanted in the patient and the implanted components are interconnected differently. In response to a control signal from the wireless remote control of the external energy-transmission device 200, the internal control unit 102 controls the accumulator 316 to deliver energy for operating the electric switch 323 to switch from an off mode, in which the battery 322 is not in use, to an on mode, in which the battery 322 supplies electric energy for the operation of the apparatus 10.

Alternatively, the electric switch 323 may be operated by energy supplied by the accumulator 316 to switch from an off mode, in which the wireless remote control is prevented from controlling the battery 322 to supply electric energy and is not in use, to a standby mode, in which the wireless remote control is permitted to control the battery 322 to supply electric energy for the operation of the apparatus 10.

It should be understood that the switch 323 and all other switches in this application should be interpreted in its broadest embodiment. This means a transistor, MCU, MCPU, ASIC, FPGA or a DA converter or any other electronic component or circuit that may switch the power on and off. Preferably the switch is controlled from outside the body, or alternatively by an implanted internal control unit.

FIG. 677 shows an embodiment identical to that of FIG. 673, except that a motor 307, a mechanical reversing device in the form of a gear box 324, and an internal control unit 102 for controlling the gear box 324 also are implanted in the patient. The internal control unit 102 controls the gear box 324 to reverse the function performed by the apparatus 10 (mechanically operated). Even simpler is to switch the direction of the motor electronically. The gear box interpreted in its broadest embodiment may stand for a servo arrangement saving force for the operation device in favor of longer stroke to act.

FIG. 678 shows an embodiment identical to that of FIG. 674 except that the implanted components are interconnected differently. Thus, in this case the internal control unit 102 is powered by the battery 322 when the accumulator 316, suitably a capacitor, activates the electric switch 323 to switch to an on mode. When the electric switch 323 is in its on mode the internal control unit 102 is permitted to control the battery 322 to supply, or not supply, energy for the operation of the apparatus 10.

FIG. 679 schematically shows conceivable combinations of implanted components of the apparatus for achieving various communication options. Basically, there are the apparatus 10, the internal control unit 102, motor or pump unit 309, and the external energy-transmission device 200 including the external wireless remote control. As already described above the wireless remote control transmits a control signal which is received by the internal control unit 102, which in turn controls the various implanted components of the apparatus.

A feedback device, preferably comprising a sensor or measuring device 325, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 325 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow.

The feedback may be sent to the internal control unit or out to an external control unit preferably via the internal control unit. Feedback may be sent out from the body via the energy transfer system or a separate communication system with receiver and transmitters.

The internal control unit 102, or alternatively the external wireless remote control of the external energy-transmission device 200, may control the apparatus 10 in response to signals from the sensor 325.

A transceiver may be combined with the sensor 325 for sending information on the sensed physical parameter to the external wireless remote control. The wireless remote control may comprise a signal transmitter or transceiver and the internal control unit 102 may comprise a signal receiver or transceiver.

Alternatively, the wireless remote control may comprise a signal receiver or transceiver and the internal control unit 102 may comprise a signal transmitter or transceiver. The above transceivers, transmitters and receivers may be used for sending information or data related to the apparatus 100 from inside the patient's body to the outside thereof.

Where the motor/pump unit 309 and battery 322 for powering the motor/pump unit 309 are implanted, information related to the charging of the battery 322 may be fed back. To be more precise, when charging a battery or accumulator with energy feedback information related to said charging process is sent and the energy supply is changed accordingly.

FIG. 680 shows an alternative embodiment wherein the apparatus 10 is regulated from outside the patient's body. The system 300 comprises a battery 322 connected to the apparatus 100 via a subcutaneous electric switch 326. Thus, the regulation of the apparatus 10 is performed non-invasively by manually pressing the subcutaneous switch, whereby the operation of the apparatus 10 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 681 shows an alternative embodiment, wherein the system 300 comprises a hydraulic fluid reservoir 313 hydraulically connected to the apparatus. Non-invasive regulation is performed by manually pressing the hydraulic reservoir connected to the apparatus. The system may include an external data communicator and an implantable internal data communicator communicating with the external data communicator. The internal communicator feeds data related to the apparatus or the patient to the external data communicator and/or the external data communicator feeds data to the internal data communicator.

FIG. 682 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 302 connected to implanted energy consuming components of the apparatus 10. Such an energy receiver 302 may include an energy source and/or an energy-transforming device. Briefly described, wireless energy is transmitted from an external energy source 304a located outside the patient and is received by the internal energy receiver 302 located inside the patient. The internal energy receiver is adapted to directly or indirectly supply received energy to the energy consuming components of the apparatus 10 via a switch 326. An energy balance is determined between the energy received by the internal energy receiver 302 and the energy used for the apparatus 10, and the transmission of wireless energy is then controlled based on the determined energy balance. The energy balance thus provides an accurate indication of the correct amount of energy needed, which is sufficient to operate the apparatus 100 properly, but without causing undue temperature rise. In FIG. 682 the patient's skin is indicated by a vertical line SK. Here, the energy receiver comprises an energy-transforming device 302 located inside the patient, preferably just beneath the patient's skin SK. Generally speaking, the implanted energy-transforming device 302 may be placed in the abdomen, thorax, muscle fascia (e.g. in the abdominal wall), subcutaneously, or at any other suitable location. The implanted energy-transforming device 302 is adapted to receive wireless energy E transmitted from the external energy-source 304a provided in an external energy-transmission device 200 located outside the patient's skin SK in the vicinity of the implanted energy-transforming device 302. As is well known in the art, the wireless energy E may generally be transferred by means of any suitable Transcutaneous Energy Transfer (TET) device, such as a device including a primary coil arranged in the external energy source 304a and an adjacent secondary coil arranged in the implanted energy-transforming device 302. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used. The amount of energy received by the implanted energy receiver may be compared with the energy used by the implanted components of the apparatus. The term “energy used” is then understood to include also energy stored by implanted components of the apparatus. A control device includes an external control unit 1004b that controls the external energy source 1004a based on the determined energy balance to regulate the amount of transferred energy. In order to transfer the correct amount of energy, the energy balance and the required amount of energy is determined by means of a determination device including an implanted internal control unit 1015 connected between the switch 326 and the apparatus 10. The internal control unit 102 may thus be arranged to receive various measurements obtained by suitable sensors or the like, not shown, measuring certain characteristics of the apparatus 10, somehow reflecting the required amount of energy needed for proper operation of the apparatus 10. Moreover, the current condition of the patient may also be detected by means of suitable measuring devices or sensors, in order to provide parameters reflecting the patient's condition. Hence, such characteristics and/or parameters may be related to the current state of the apparatus 10, such as power consumption, operational mode and temperature, as well as the patient's condition reflected by parameters such as; body temperature, blood pressure, heartbeats and breathing. Other kinds of physical parameters of the patient and functional parameters of the device are described elsewhere. Furthermore, an energy source in the form of an accumulator 316 may optionally be connected to the implanted energy-transforming device 302 via the control unit 102 for accumulating received energy for later use by the apparatus 10. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 10, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics. For example, battery characteristics may be measured on a regular basis to determine the current state of the battery, which then may be stored as state information in a suitable storage means in the internal control unit 102. Thus, whenever new measurements are made, the stored battery state information can be updated accordingly. In this way, the state of the battery can be “calibrated” by transferring a correct amount of energy, so as to maintain the battery in an optimal condition. Thus, the internal control unit 102 of the determination device is adapted to determine the energy balance and/or the currently required amount of energy, (either energy per time unit or accumulated energy) based on measurements made by the above-mentioned sensors or measuring devices of the apparatus 10, or the patient, or an implanted energy source if used, or any combination thereof. The internal control unit 102 is further connected to an internal signal transmitter 327, arranged to transmit a control signal reflecting the determined required amount of energy, to an external signal receiver 304c connected to the external control unit 304b. The amount of energy transmitted from the external energy source 304a may then be regulated in response to the received control signal. Alternatively, the determination device may include the external control unit 304b. In this alternative, sensor measurements can be transmitted directly to the external control unit 304b wherein the energy balance and/or the currently required amount of energy can be determined by the external control unit 304b, thus integrating the above-described function of the internal control unit 102 in the external control unit 304b. In that case, the internal control unit 102 can be omitted and the sensor measurements are supplied directly to the internal signal transmitter 327 which sends the measurements over to the external signal receiver 304c and the external control unit 304b. The energy balance and the currently required amount of energy can then be determined by the external control unit 304b based on those sensor measurements. Hence, the present solution according to the arrangement of FIG. 682 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus. The internal signal transmitter 327 and the external signal receiver 304c may be implemented as separate units using suitable signal transfer means, such as radio, IR (Infrared) or ultrasonic signals. Alternatively, the internal signal transmitter 327 and the external signal receiver 304c may be integrated in the implanted energy-transforming device 302 and the external energy source 304a, respectively, so as to convey control signals in a reverse direction relative to the energy transfer, basically using the same transmission technique. The control signals may be modulated with respect to frequency, phase or amplitude. Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off. In implementing this system in the arrangement of FIG. 682, the switch 326 is either separate and controlled by the internal control unit 102, or integrated in the internal control unit 102. It should be understood that the switch 326 should be interpreted in its broadest embodiment. This means a transistor, MCU, MCPU, ASIC FPGA or a DA converter or any other electronic component or circuit that may switch the power on and off. To conclude, the energy supply arrangement illustrated in FIG. 682 may operate basically in the following manner. The energy balance is first determined by the internal control unit 102 of the determination device. A control signal reflecting the required amount of energy is also created by the internal control unit 102, and the control signal is transmitted from the internal signal transmitter 327 to the external signal receiver 304c. Alternatively, the energy balance can be determined by the external control unit 304b instead depending on the implementation, as mentioned above. In that case, the control signal may carry measurement results from various sensors. The amount of energy emitted from the external energy source 304a can then be regulated by the external control unit 304b, based on the determined energy balance, e.g. in response to the received control signal. This process may be repeated intermittently at certain intervals during ongoing energy transfer, or may be executed on a more or less continuous basis during the energy transfer. The amount of transferred energy can generally be regulated by adjusting various transmission parameters in the external energy source 304a, such as voltage, current, amplitude, wave frequency and pulse characteristics. This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized. This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor. With reference to FIG. 683, although wireless transfer of energy for operating the apparatus has been described above to enable non-invasive operation, it will be appreciated that the apparatus can be operated with wire bound energy as well. Such an example is shown in FIG. 683, wherein an external switch 326 is interconnected between the external energy source 304a and an operation device, such as an electric motor 307 operating the apparatus 10. An external control unit 304b controls the operation of the external switch 326 to effect proper operation of the apparatus 10.

FIG. 684 illustrates different embodiments for how received energy can be supplied to and used by the apparatus 10. Similar to the example of FIG. 682, an internal energy receiver 302 receives wireless energy E from an external energy source 304a which is controlled by a transmission control unit 304b. The internal energy receiver 302 may comprise a constant voltage circuit, indicated as a dashed box “constant V” in the figure, for supplying energy at constant voltage to the apparatus 10. The internal energy receiver 302 may further comprise a constant current circuit, indicated as a dashed box “constant C” in the figure, for supplying energy at constant current to the apparatus 10. The apparatus 10 comprises an energy consuming part 10a, which may be a motor, pump, restriction device, or any other medical appliance that requires energy for its electrical operation. The apparatus 10 may further comprise an energy storage device 10b for storing energy supplied from the internal energy receiver 302. Thus, the supplied energy may be directly consumed by the energy consuming part 10a, or stored by the energy storage device 10b, or the supplied energy may be partly consumed and partly stored. The apparatus 10 may further comprise an energy stabilizing unit 10c for stabilizing the energy supplied from the internal energy receiver 302. Thus, the energy may be supplied in a fluctuating manner such that it may be necessary to stabilize the energy before consumed or stored. The energy supplied from the internal energy receiver 302 may further be accumulated and/or stabilized by a separate energy stabilizing unit 328 located outside the apparatus 10, before being consumed and/or stored by the apparatus 10. Alternatively, the energy stabilizing unit 328 may be integrated in the internal energy receiver 302. In either case, the energy stabilizing unit 328 may comprise a constant voltage circuit and/or a constant current circuit.

It should be noted that FIG. 682 and FIG. 684 illustrate some possible but non-limiting implementation options regarding how the various shown functional components and elements can be arranged and connected to each other. However, the skilled person will readily appreciate that many variations and modifications can be made within the scope of the present invention.

FIG. 685 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window. The schematic FIG. 685 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 668; the transmitting parts of the system are excluded. The implementation of the general concept of energy balance and the way the information is transmitted to the external energy transmitter can of course be implemented in numerous different ways. The schematic FIG. 640 and the above described method of evaluating and transmitting the information should only be regarded as examples of how to implement the control system.

### Circuit Details

In FIG. 685 the symbols Y1, Y2, Y3 and so on symbolize test points within the circuit. The components in the diagram and their respective values are values that work in this particular implementation which of course is only one of an infinite number of possible design solutions. Energy to power the circuit is received by the energy receiving coil L1. Energy to implanted components is transmitted in this particular case at a frequency of 25 kHz. The energy balance output signal is present at test point Y1. Those skilled in the art will realize that the above various embodiments of the system could be combined in many different ways. For example, the electric switch 306 of FIG. 668 could be incorporated in any of the embodiments of FIGS. 671-677, the hydraulic valve shifting device 314 of FIG. 671 could be incorporated in the embodiment of FIG. 670, and the gear box 324 could be incorporated in the embodiment of FIG. 669. Please observe that the switch simply could mean any electronic circuit or component. The embodiments described in connection with FIGS. 682, 684 and 685 identify a method and a system for controlling transmission of wireless energy to implanted energy consuming components of an electrically operable apparatus. Such a method and system will be defined in general terms in the following. A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance. The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference. When controlling the energy transmission, the amount of transmitted wireless energy may be decreased if the detected energy balance change implies that the energy balance is increasing, or vice versa. The decrease/increase of energy transmission may further correspond to a detected change rate. The amount of transmitted wireless energy may further be decreased if the detected energy difference implies that the received energy is greater than the used energy, or vice versa. The decrease/increase of energy transmission may then correspond to the magnitude of the detected energy difference. As mentioned above, the energy used for the medical device may be consumed to operate the medical device, and/or stored in at least one energy storage device of the medical device. When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters. When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance. When the derivative is determined over time of a measured electrical parameter related to the amount of consumed and/or stored energy, the derivative may be determined for a monitored voltage and/or current related to the energy balance. The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses. A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied. When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency. The circuit formed by the first electric circuit and the external energy source may have a first characteristic time period or first time constant, and when effectively varying the transmitted energy, such frequency time period may be in the range of the first characteristic time period or time constant or shorter. A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

FIGS. 686-S44 show in more detail block diagrams of four different ways of hydraulically or pneumatically powering an implanted apparatus according to the invention.

FIG. 686 shows a system as described above with. The system comprises an implanted apparatus 100 and further a separate regulation reservoir 1013, a one way pump 1009 and an alternate valve 1014.

FIG. 687 shows the apparatus 10 and a fluid reservoir 1013. By moving the wall of the regulation reservoir or changing the size of the same in any other different way, the adjustment of the apparatus may be performed without any valve, just free passage of fluid any time by moving the reservoir wall.

FIG. 688 shows the apparatus 10, a two way pump 1009 and the regulation reservoir 1013.

FIG. 689 shows a block diagram of a reversed servo system with a first closed system controlling a second closed system. The servo system comprises a regulation reservoir 1013 and a servo reservoir 1050. The servo reservoir 1050 mechanically controls an implanted apparatus 10 via a mechanical interconnection 1054. The apparatus has an expandable/contactable cavity. This cavity is preferably expanded or contracted by supplying hydraulic fluid from the larger adjustable reservoir 1052 in fluid connection with the apparatus 10. Alternatively, the cavity contains compressible gas, which can be compressed and expanded under the control of the servo reservoir 1050. The servo reservoir 1050 can also be part of the apparatus itself. In one embodiment, the regulation reservoir is placed subcutaneous under the patient's skin and is operated by pushing the outer surface thereof by means of a finger. This system is illustrated in FIGS. 690a-c. In FIG. 690a, a flexible subcutaneous regulation reservoir 1013 is shown connected to a bulge shaped servo reservoir 1050 by means of a conduit 1011. This bellow shaped servo reservoir 1050 is comprised in a flexible apparatus 10. In the state shown in FIG. 690a, the servo reservoir 1050 contains a minimum of fluid and most fluid is found in the regulation reservoir 1013. Due to the mechanical interconnection between the servo reservoir 1050 and the apparatus 10, the outer shape of the apparatus 100 is contracted, i.e., it occupies less than its maximum volume. This maximum volume is shown with dashed lines in the figure.

FIG. 690b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 100 so that it occupies its maximum volume, thereby stretching the stomach wall (not shown), which it contacts.

The regulation reservoir 1013 is preferably provided with means 1013a for keeping its shape after compression. This means, which is schematically shown in the figure, will thus keep the apparatus 100 in a stretched position also when the user releases the regulation reservoir. In this way, the regulation reservoir essentially operates as an on/off switch for the system.

An alternative embodiment of hydraulic or pneumatic operation will now be described with reference to FIGS. 671 and 672a-c. The block diagram shown in FIG. 691 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 100 having an expandable/contactable cavity is in turn controlled by the larger adjustable reservoir 1052 by supply of hydraulic fluid from the larger adjustable reservoir 1052 in fluid connection with the apparatus 10.

An example of this embodiment will now be described with reference to FIG. 692a-c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 676a, the servo reservoir 1050 contains a minimum of fluid and most fluid is found in the regulation reservoir 1013.

The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 10. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 10. In other words, in this reversed servo, a small volume in the regulation reservoir is compressed with a higher force and this creates a movement of a larger total area with less force per area unit. Like in the previous embodiment described above with reference to FIGS. 690a-c, the regulation reservoir 1013 is preferably provided with means 1013a for keeping its shape after compression. This means, which is schematically shown in the figure, will thus keep the apparatus 100 in a stretched position also when the user releases the regulation reservoir. In this way, the regulation reservoir essentially operates as an on/off switch for the system.

FIG. 641 shows a system of the invention, with the skin of a patient being shown as “SK”, and with an apparatus 30 being implanted into a patient, “Int”, and with other details on the outside of the patient, “Ext”

Besides the apparatus 30, the implanted equipment comprises an energy transforming device 302 as described above, a battery 1022 and, as an alternative or complement, an accumulator 1016, with both the energy transforming device and the battery/accumulator being controlled by the control device 1015. The “external equipment” comprises a remote control, which is shown as possibly comprising two parts, i.e. a transmitter or transceiver for transmitting and possibly receiving energy to/from the device 302, and a remote control 1, which may be integrated int one physical unit together with the transmitter or transceiver.

FIGS. 650-652 show how the “flat plate” embodiment can be powered so that the moving part 51 is given kinetic energy in a plurality of steps: As shown, the casing 61, or to be more exact, the part or parts of the casing 61 which is arranged to house the moving part 51 outside of the blood vessel 52 is equipped with one or more coils 701-709, 701′-709′, which are arranged to be energized by alternating current, AC, so as to interact with one or more magnets 710-712 magnets arranged on the moving part 51. The coils will thus in their interaction with the magnets cause the movement of the moving part 51.

As shown, the coils are suitably arranged in a line along one or both sides of the intended movement of the plate 51. The coils can then be energized by running AC current through them stepwise, as the plate moves in its intended course due to the interaction between the magnets and the coils. Naturally, an arrangement with one coil and a plurality of magnets, as well as an arrangement with one magnet and a plurality of coils can also be envisioned within the scope of the present invention.

The embodiments with one or more moving parts which move about hinges can be powered stepwise to a closing and/or opening movement in a manner which is shown in FIG. 653: On each moving part 31-33, there is attached a number of magnets 804-806, and on the casing O′ of the valve 30, there is arranged coils 801-803, suitably one coil per group of magnets, although other combinations are also possible, i.e. one magnet in combination with a group of coils, and other such combinations. The magnets 804-806 are arranged so that they will be affected electromagnetically when an AC current is passed through the coil or coils and will cause the moving part 31-33 to perform its opening or closing movement. As the moving part performs its movement, the magnets will successively pass before the coil, thus giving the moving part kinetic energy stepwise.

The arrangement for running AC through the coils is not shown in the drawings nor described here, since such an arrangement lies within the scope of what a man skilled in the field has knowledge of.

As mentioned, the following embodiment can, in a non-exclusive manner, be envisioned for a closing mechanism which comprises one or more interacting magnets and coils:
the closing mechanism comprises at least two magnets, said closing mechanism being adapted to receive said additional pulses to affect a different magnet or a different group of magnets than said first pulse.
the closing mechanism comprises a coil which is adapted to be energized so as to cause said movement of the closing mechanism.
the closing mechanism comprises a coil which is adapted to be energized stepwise with two or more energy pulses so as to cause said movement of the closing mechanism.
the closing mechanism comprises a plurality of coils which are adapted to be energized stepwise so as to cause said movement of the closing mechanism.

Thus, one or both of the opening and closing movements will suitably take place as the result of being powered by a mechanism, such as the one shown in drawings 650-653 and described in connection to those drawings. Such opening and closing suitably takes place as the result of a sensor sensing that a parameter of the patient's body has reached a threshold level, as a result of which the opening or closing movement is initiated and takes place. As an option to a powered opening mechanism, as has been described above, the artificial valve in the “flat plate” embodiment comprises a biasing mechanism which strives to open the valve.

The invention also discloses a method as follows:

A. A method of surgically placing a valve in a patient's heart or blood vessel via a laparoscopic thoracic approach, the method comprising the steps of:
inserting a needle or a tube like instrument into the thorax of the patient's body,
using the needle or a tube like instrument to fill the thorax with gas thereby expanding the thoracic cavity,
placing at least two laparoscopic trocars in the patient's body,
inserting a camera through one of the laparoscopic trocars into the thorax,
inserting at least one dissecting tool through one of said at least two laparoscopic trocars and dissecting an intended placement area of the patient,
placing the valve in any part of the blood stream in the thorax, and
placing and connecting an implanted energy receiver or source of energy for powering the valve to perform at least one of the following method steps;
at least partly closing and at least partly opening of the valve.

B. An operation method for surgically placing a valve in a patient's heart or blood vessel, the method comprising the steps of:
cutting the patient's skin,
opening the thoracic cavity,
dissecting a placement area where to place the valve inside a blood stream in the heart, or the aorta or inside the pulmonary artery of the human patient,
placing the a valve in the placement area in any part of the blood stream in the thorax, and
placing and connecting an implanted energy receiver or a source of energy for powering the valve to perform at least one of the following method steps;
at least partly closing and at least partly opening of the valve.

C. A method of surgically placing a valve in a patient's heart or blood vessel via a laparoscopic abdominal approach, the method comprising the steps of:
inserting a needle or a tube like instrument into the abdomen of the patient's body,
using the needle or a tube like instrument to fill the thorax with gas thereby expanding the abdominal cavity,
placing at least two laparoscopic trocars in the patient's abdomen
inserting a camera through one of the laparoscopic trocars into the abdomen,
inserting at least one dissecting tool through one of said at least two laparoscopic trocars and
dissecting and creating an opening in the diaphragm muscle,
dissecting an intended placement area of the patient through said opening,
placing the valve in any part of the blood stream in the thorax, and
placing and connecting an implanted energy receiver or source of energy for powering the valve to perform at least one of the following method steps;
at least partly closing and at least partly opening of the valve.

D. An operation method for surgically placing a valve in a patient's heart or blood vessel, the method comprising the steps of:
cutting the patient's skin,
opening the abdominal cavity,
dissecting and creating an opening in the diaphragm muscle,
dissecting a placement area where to place the valve inside a blood stream in the heart, or the aorta or inside the pulmonary artery of the human patient through said opening,
placing the a valve in the placement area, and
placing and connecting an implanted energy receiver or a source of energy for powering the valve to perform at least one of the following method steps;
at least partly closing and at least partly opening of the valve.

E. An operation method for surgically placing a valve in a patient's heart or blood vessel, via inguinal key-hole surgery approach, the method comprising the steps of
cutting the patients skin,
inserting a needle or a tube like instrument into the inguinal area of the patient's body,
using the needle or a tube like instrument to fill a cavity with gas thereby expanding the cavity,
placing at least two laparoscopic trocars in the patient's cavity
inserting a camera through one of the trocars into the cavity,
inserting at least one dissecting tool through one of said at least two trocars and
dissecting the area of the femoral artery,
inserting a tube like instrument into the femoral artery of the patient's body,
inserting said valve into the femoral artery,
using said instrument to guide said valve through the femoral artery to the aorta or heart of the patient,
releasing the valve inside of a blood vessel or heart
placing said valve in the blood vessel or heart,
placing and connecting an implanted energy receiver or a source of energy for powering the valve to perform at least one of the following method steps;
at least partly closing and at least partly opening of the valve.

F. An operation method for surgically placing a valve in a patient's heart or blood vessel, via a inguinal approach, the method comprising the steps of
cutting the patients skin,
dissecting the inguinal region,
dissecting the area of the femoral artery,
inserting a tube like instrument into the femoral artery of the patient's body,
using said instrument to guide said rotating body through the femoral artery and the aorta to the blood vessel or heart,
releasing the valve inside of the heart or blood vessel,
placing said valve in the blood vessel or heart,
placing and connecting an implanted energy receiver or a source of energy for powering the valve to perform at least one of the following method steps;
at least partly closing and at least partly opening of the valve.

G. In one embodiment according to any of items A-F, the step of placing the valve additionally comprises the step of
placing a drive unit for at least partly powering the valve movements in the placement area, inside the blood stream of the blood vessel, inside the heart, or the aorta or inside the pulmonary artery of the patient,
supplying energy from said drive unit to said valve causing movement of said valve.

H. In one embodiment according to any of items A-F, the step of placing the valve additionally comprises the step of:
placing a drive unit for at least partly powering the valve movements in the placement area, outside the blood stream of the blood vessel, outside the heart, or the aorta or outside the pulmonary artery of the patient, placing said drive unit on the outside of said valve,
supplying energy from said drive unit to said valve causing movement of said valve.

I. In one embodiment according to items I or H, the step of supplying energy from said drive unit to said valve, causing movement of said valve, additionally comprises the step of:
supplying wireless or magnetic energy from said drive unit to said valve, causing movement of said valve.

J. In one embodiment according to any of items G-I, the method additionally comprises the step of:
connecting the drive unit with the energy receiver or source of energy for powering said drive unit.

K. In one embodiment according to any of items A-D and H, for parts of the valve placed outside the blood stream, combining with the method according to one or more of claims E-G for parts of the valve placed inside the blood stream.

L. In one embodiment according to item J, said drive unit placed outside the blood stream comprises a stator, and the part of the valve placed inside the blood stream comprises a rotor, wherein said stator supplies wireless energy to said part of the valve placed inside the blood stream, causing rotational movement of at least a part of said drive unit.

M. In one embodiment according to item L, the drive unit further comprises both said rotor adapted to be placed outside the blood stream, said rotor comprising a magnetic coupling for driving at least a part of the valve placed inside the blood stream with rotational energy, the method further comprising the steps of:
placing said stator and rotor on the outside of said valve including a magnetic coupling in the placement area, wherein said rotor comprises said magnetic coupling, adapted to be magnetically connecting to said valve placed inside the blood stream,
supplying energy to said stator to rotate said rotor and thereby rotating said valve, thereby
causing, through the magnetic coupling, rotating movement of said valve.

N. In one embodiment according to any of items A-M, an opening is performed from the abdomen through the thoracic diaphragm for placing the energy receiver or energy source in the abdomen.

O. In one embodiment according to any of items C, D and N, said opening is performed in the thoracic diaphragm at the place where the pericardium is attached to the thoracic diaphragm.

P. In one embodiment according to any of items A-O, the valve or drive unit uses energy, direct or indirect, from an external energy source, supplying energy non-invasively, without any penetration through the patient's skin to power the valve or drive unit.

Q. In one embodiment according to any of items A-H, said valve or drive unit is connected to an internal energy source via a cable, the method of placement further comprising;
dissecting and placing a wire connected to the valve or drive unit into the right atrium of the heart and further up in the venous blood vessel system,
exiting the system in or closer to the subcutaneous area, such as in the vena subclavia, vena jugularis or vena brachialis
placing an internal energy source in the subcutaneous area or close thereto or in the thorax or abdomen,
supplying from an external energy source energy non-invasively, without any penetration through the patient's skin to power the internal energy source for indirect or direct power the valve or drive unit.

R. In one embodiment according to any of items A-H, the method of placement further comprises;
placing an electrode in the right atrium or ventricle of the heart
placing the wire to the electrode via the right atrium of the heart and further up in the venous blood vessel system,
exiting the blood vessel system in or closer to the subcutaneous area, such as in the vena subclavia, vena jugularis or vena brachialis,
placing an internal control unit in the subcutaneous area or close thereto or in the thorax or abdomen, the method further comprising at least one of the following steps;
receiving sensor input relating to electrical pulses or muscle contractions of the heart or
transmitting energy pulses from said electrode for controlling heart contractions,
coordinating the valve or drive unit.

In various embodiments, the artificial valve also exhibits the following features:

A. The artificial valve is adapted to pass through a laparoscopic trocar in the patient's body.

B. The artificial valve of item A is adapted to pass through an opening in the diaphragm muscle from the abdominal side.

C. The artificial valve of item A is adapted to be inserted into the femoral artery and further adapted to be released inside of the heart or blood vessel.

D. The artificial valve of item A comprises a drive unit for at least partly powering the valve movements, adapted to be placed inside the blood stream including a blood vessel or heart.

E. The artificial valve of item A comprises a drive unit for at least partly powering the valve movements, adapted to be placed outside the blood stream including a blood vessel or heart.

F. The artificial valve of item D or E, wherein said drive unit is adapted to supply wireless or magnetic energy, said valve being adapted to receive said wireless or magnetic energy to cause movements of said valve.

G. The artificial valve of item D or E, wherein said drive unit comprises a stator, adapted to be placed outside the blood stream, the blood vessel or heart, and further comprising a rotor adapted to be placed inside the blood stream, wherein said stator is adapted to supply wireless or magnetic energy to the rotor placed inside the blood stream, causing movements of at least a part of said valve placed inside the blood stream.

H. The artificial valve of item D or E, wherein said drive unit comprises a stator and a rotor, adapted to be placed outside the blood stream, the blood vessel or heart, said rotor comprising a magnetic coupling for driving at least a part of the valve placed inside the blood stream with kinetic energy.

I. The artificial valve of item A, wherein an energy receiver or energy source is adapted to be placed in the abdomen.

J. The artificial valve of item D or E, comprising an electric wire adapted to connect said valve or drive unit to an internal energy source, said wire adapted to pass into the right atrium of the heart and further up in the venous blood vessel system, exiting the blood vessel system in or closer to the subcutaneous area, wherein said internal energy source is adapted to be connected to said wire via the subcutaneous area.

K. The artificial valve of item A, comprising;
an internal control unit,
a sensor sensing physiological electrical pulses or muscle contractions of the heart,
wherein said control unit controls said valve according to the sensed information.

L. The artificial valve of item J:
in which said internal energy source comprises an internal control unit adapted to transmit energy pulses to said electrode for achieving and controlling heart contractions, wherein said control unit is adapted to coordinate the valve or drive unit.

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating a device for constricting a luminary organ, examples of such a device for constricting a luminary organ will now be described.

### T: Constriction of a Luminary Organ

System including an implantable control unit/controller for controlling and communicating with at least one powered/operable implant has been described herein. Further, systems, methods and units for communication with the control unit/controller and communication between external devices connected to, or involved in, the control or communication with the implantable control unit/controller have been described. Also, embodiments of implantable devices for fixation and housing of implantable control units/controllers for controlling and communicating with at least one powered/operable implant, as well as for fixation and housing of operation devices for operating elements of powered/operable implantable medical devices have been described. The control unit/controller, control systems, communication systems, housing/fixation units and methods could be used for controlling, communicating with and/or operating an anal incontinence treatment apparatus, examples of such an anal incontinence treatment apparatus valves will now be described.

FIG. 658 schematically illustrates an implant 100 according to any of the above-described embodiments, when implemented as an anal incontinence treatment apparatus. Thus, an implanted constriction device 10 may be arranged to extend around a portion of an intestine 19, such as the rectum or colon, of a patient suffering from anal incontinence, to allow the rectum or colon to be squeezed to restrict the fecal passageway. The anal incontinence treatment apparatus may further comprise a control unit 102, which may be connected to the constriction device 10 via a fluid conduit 109. The control unit 102 may further be communicatively connected to an external control unit 200 for transmitting an information signal and/or energy to the implant.

FIG. 659a schematically illustrates an implant according to any of the above-described embodiments, when implemented as a constriction device for constricting the intestine at a region of a stoma of the patient. Thus, similar to the implementation in FIG. 658 an implanted constriction device 10 may be arranged to extend around a portion of an intestine 19 to allow the intestine to be squeezed to restrict the fecal passageway. The device may further comprise a control unit 102 connected to the constriction device 10 via a fluid conduit 109. The control unit 102, which may be arranged subcutaneously, may further be communicatively connected to an external control unit 200 as previously described.

FIG. 659b schematically illustrates an alternative arrangement of a constriction device 10, which may be similarly configured as the constriction device 10 of FIG. 659a, when arranged to extend around the colon at a position close to the rectum of the patient so as to control a fecal passageway.

FIG. 659c schematically illustrates a further alternative arrangement to the one disclosed in FIG. 659a, wherein the constriction device 10 is arranged to extend around a graft forming an artificial stoma 39a exiting the body in the abdominal region. As shown in the present figure, the artificial stoma 39a exits an abdominal wall 39b of the patient. The present example thus differs from the implementation in FIG. 659a in that the constriction device 10 is arranged to squeeze on the graft 39a rather than on the intestine 19 itself.

FIGS. 660a and 660b schematically illustrate an implant according to any of the above-described embodiments, when implemented as a fertility control device for controlling a flow of sperm through a vas deference of a male patient. Thus, in the present example a first constriction device 10a and a second constriction device 10b may be arranged to extend around a respective lumen, being a vas deference, to allow the respective vas deference to be squeezed and thereby restrict the sperm passageway. The device may further comprise a control unit 102 as previously described.

FIGS. 661a and 661b schematically illustrates an implant according to any of the above-described embodiments, when implemented as a constriction device 10 for constricting a blood vessel 34 of the patient. The blood vessel 34 may for example be a pulmonary artery 34 of the heart. FIG. 661a illustrates an implementation wherein the constriction device 10 is arranged to constrict the pulmonary artery 34 at a position close to the pulmonic valve, between the valve and the pulmonary artery bifurcation, whereas FIG. 661b illustrates another implementation wherein a first constriction device 10 and a second constriction device 10 is arranged after the bifurcation, at the branch of the pulmonary artery 34 leading to the left lung and the right lung, respectively.

As illustrated in FIG. 661a the constriction device 10 may be connected to a control unit 102, which may be arranged subcutaneously. The constriction device 10 may be operatively connected to the control unit via a fluid conduit 109. Further, the control unit 102 may be connected to a transceiver 308 for receiving information and/or energy from an external unit 200.

FIG. 662 schematically illustrates an implant according to any of the above-described embodiments, when implemented as an impotence treatment apparatus. Thus, an implanted constriction device 10 may be arranged to extend around a portion of a blood vessel 34, transporting venous blood to and from an erectile tissue of a male patient, to allow the venous blood flow leading from the erectile tissue to be restricted to promote the engorgement of the erectile tissue. The impotence treatment apparatus may further comprise a control unit 102, which may be connected to the constriction device 10 via a fluid conduit 109. The control unit 102 may further be communicatively connected to an external control unit 200 as previously described.

FIG. 663 schematically illustrates an implant according to any of the above-described embodiments, when implemented as a hypertension treatment apparatus. Thus, an implanted constriction device 10 may be arranged to extend around a portion of a bile duct 34b of the patient, in the present example between the gall bladder 35 and the bifurcation to the main duct 34a, to allow the passageway from the gall bladder 35 to be restricted. By controlling the constriction of the bile duct 34b hypertension or hypotension can be treated. The implant may further comprise a control unit 102, which may be connected to the constriction device 10 via a fluid conduit 109, as well as to an external control unit 200.

FIGS. 664a and 664b schematically illustrate an implant according to any of the above-described embodiments, when implemented in a similar manner as the implant illustrated in FIG. 662. The present implementation however differs in that the implant is implanted in a female patient. Thus, a first implanted constriction device 10a and a second implanted constriction device 10b may be arranged to extend around a portion of a respective blood vessel 204, transporting venous blood to and from an erectile tissue 205 of the female patient, to allow the venous blood flow leading from the erectile tissue 205 to be restricted to promote the engorgement of the erectile tissue 205. The implant may further comprise a control unit 102, which may be arranged subcutaneously. The constriction devices 10a, 10b may be operatively connected to the control unit via a fluid conduit 109. Further, the control unit 102 may be connected to a transceiver 308 for receiving information and/or energy from an external unit 200.

FIGS. 665a and 665b schematically illustrate an implant according to any of the above-described embodiments, when implemented as a constriction device 10 for constricting a blood vessel 34 of the patient. The blood vessel 34 may for example be the aorta, and the constriction device 10 may for example be arranged on the abdominal aorta 10. In the present example, the constriction device 10 may hence be implemented as an aneurysm treatment apparatus, arranged to extend around an aneurysm 34′ on the blood vessel 34. The constriction device 10 may be used for counteracting or contracting the aneurysm so as to reduce the risk of the aneurysm rupturing. As illustrated in FIG. 665b the implant may further comprise an implantable sensor 106, which may be arranged inside the blood vessel, for providing a signal relating to e.g. the blood pressure in the aneurysm to a control unit 102 so as to allow the constricting pressure to be regulated accordingly. The implant may further comprise a pump 104 for moving fluid from a reservoir 107 to the constriction device 10. Further, the control unit 102 may be configured to communicate with an external unit 200.

### U: Stretch

With reference to FIG. 693a, an apparatus for treating obesity is disclosed. A volume filling device 10 is invaginated in the stomach wall and fixed to a position by a fixation, such as sutures or staples 14a, for example a direct or indirect fixation to the diaphragm muscle or associated muscles may be provided. As an alternative a direct or indirect fixation to the esophagus above and close to the angle of His can be provided. In this alternative embodiment, the volume filling device 10 rests in a position against stomach wall of the fundus when implanted and which also fills a volume above the cardia area between the cardia and the diaphragm muscle so that the cardia is prevented from slipping up into the thorax cavity, whereby reflux disease is prevented.

Such a volume filling device 10 may be used for keeping electronics and/or an energy source and/or hydraulic fluid. Hydraulic fluid from that device may be distributed to several smaller inflatable device areas to vary the stretching area from time to time avoiding any possible more permanent stretching effect of the stomach wall. Even mechanically several stretching areas may be used.

The volume of the volume filling device 10 may be in fluid connection with one or more preferably smaller inflatable devices or chambers 50 via respective fluid connection devices, as illustrated. These chambers are adapted to communicate with fluid or air being moved between the chambers.

Thus, the volume filling device may reduce the size of the food cavity and alternatively treat reflux disease, and the one or several small chambers 50 are adapted to function as the inflatable devices to treat obesity, wherein the main chamber is adapted to communicate with fluid or air to the small chambers causing a stretching effect in the stomach wall thereby further treating obesity. With reference to FIGS. 693b and 693c, a device for treating obesity is disclosed. The device includes a stretching device which may comprise a first and a second engaging part, the first part being adapted to be engaged to a first area of the stomach wall, and the second part being adapted to be engaged to a second area of the stomach wall. The stretching device is thereby adapted to stretch a portion of the stomach wall between the first area and the second area. The stretching device may comprise a motor, such as an implantable electrical motor, which in turn may operate at least one joint to move the joint to stretch the stomach wall portion.

The operable stretching device 110 may be provided with an inlet port. The stretching device 110 is invaginated in the stomach wall 12 and the inlet port is available for connection to a tube or the like from the abdominal area of the patient. The tube or conduit 18 can preferably be connected to a control unit 42 or an injection port 1001.

The stretching device 110 may comprise a first part 110a adapted to have a first fixation at a first position on the stomach wall 12 and a second part 110b adapted to have a second fixation at a second position on the stomach wall 12. These fixation parts 110a,b, which preferably have an essentially round shape and preferably are adapted to be invaginated in the stomach wall 12, are attached to the distal end of a respective leg 211, which in turn are attached at their respective proximal end to an operation device, such as a motor 40.

The motor may be a hydraulic motor, comprising a hydraulic piston, which is connected to a manual operation device 1001. The hydraulic piston affects the legs through their connection with a joint 212 placed in the extremity of the leg. The stretching device 110 is enclosed in a housing 214 protecting the device from the in growth of fibrotic tissue which potentially could damage the function of said device 110. The motor may be a pneumatic motor or an electrical motor, alternatively.

The stretching device 110 is adapted to increase the distance between the first position and the second position on the stomach wall 12, thereby stretching the stomach wall 12. The first and/or second parts 110a, 110b are adapted to at least partly be invaginated in the stomach wall 12 with stomach-to-stomach sutures or staplers 14 holding the fixation portions 110a,b in place in suspension in relation to the stomach wall 12.

The first and second positions may be sutured or fixated to the stomach wall in many possible ways and the invention covers all possibilities to distend the stomach wall by moving two portions of the stomach wall away from each other and thereby first fixating the device to at least two positions on the stomach wall.

However, the soft suspended connection to the stomach wall 12 where fibrotic stomach-to-stomach tissue helps to give a long term stable position is to prefer. Merely expanding an invaginated part of the stomach also stretches the stomach wall 12, which also may be achieved both mechanically, hydraulically, pneumatically and both being powered with a motor or pump or by manual force. Any kind of mechanical construction may be used and the mechanical embodiment disclosed is one example.

Any mechanical construction driven by mechanically or hydraulically or any pneumatic construction may be used. Any motor or any pump or moving material changing form when powered may be used to achieve the simple goal of stretching a part of the stomach wall by moving at least two portions of the stomach wall away from each other. The stretching device may be controlled from an implantable control assembly 42 to which sensor input may be received from a sensor 201, wirelessly or via a wire 202. The stretching device is then regulated through the conduit 18 using a pump 44, connected to at least one fluid reservoir 16, 46, and powered from an energy transforming member 30 connected to an receiver of wireless energy 205, placed under the skin 36, or an implantable energy source 70, such as a rechargeable battery. In view of the above, control of the stretching device may be manual and/or automatic via a control unit 1001. With reference to FIG. 693d, a device for treating obesity of a patient is shown, comprising a stretching device. It is to be understood that even though the illustrated embodiment utilizes manual control for operating the stretching device, also automatic control is possible, as disclosed in conjunction with FIG. 693c. The device comprises a stretching device 10 implanted in a human patient. The stretching device is invaginated in the stomach wall 12 of the patient's stomach 12 and the body of the stretching device is shaped to rest against the wall 12a of the stomach 12 and further has an outer surface suitable to rest against this wall 12a. This means that the stretching device 10 preferably has an essentially round shape to not damage the stomach wall. However, the stomach wall 12 is strong so many different shapes and forms may be used.

The stretching device 10 can be fixed to the wall 12a of the stomach 12 in a number of different ways. After invagination, a number of stomach-to-stomach sutures or staplers 14 are applied to keep the invagination in the short term. This allows growth of human tissue, keeping the invagination in the long term. By enlarging the size of the stretching device, the stomach wall 12a surrounding the stretching device 10 is stretched since the circumference of the stretching device 10 is increased. By this stretching, receptors in the stomach wall indicate that the stomach is full, thereby creating a feeling of satiety to the patient. Correspondingly, when the stretching device 10 is contracted, the receptors indicate that the stomach is not full, thereby returning the feeling of hunger. The expansion and contraction of the stretching device 10 can be performed under direct control of the patient. Alternatively, the expansion and contraction can be performed according to a pre-programmed schedule.

FIG. 693d further shows a reversed servo system which comprises a regulation reservoir 16 and a servo reservoir 90. The servo reservoir 90 hydraulically controls a stretching device 10 via a conduit 18. It is understood that a skilled person is in the position of combining steps, changing the order of steps, and combining elements of the different embodiments without inventive effort, and without departing from the scope as defined in the description and claims.

### V: Anal Incontinence Treatment

FIG. 699A schematically shows a very simple embodiment of the anal incontinence apparatus of the aspect having some parts implanted in a patient and other parts located outside the patient's body. Thus, in FIG. 699A all parts placed to the right of the patient's skin 2 are implanted and all parts placed to the left of the skin 2 are located outside the patient's body. The apparatus of FIG. 699A comprises an implanted operable restriction device 4, which engages the patient's colon (or alternatively engages the rectum) to form a restricted fecal passageway in the colon. The restriction device 4 is capable of performing a reversible function, i.e. to enlarge and reduce the cross-sectional area of the fecal passageway, so that the restriction device 4 works as an artificial sphincter. An implanted energy transforming means 6 is adapted to supply energy consuming components of the restriction device 4 with energy via a power supply line 12. An external energy transmission means 10 includes a wireless remote control transmitting a wireless signal which is received by a signal receiver incorporated in the implanted energy transforming means 6. The implanted energy transforming means 6 transforms energy from the wireless signal into electric energy which is supplied via the power supply line 12 to the restriction device 4, which energy causes portions of the device 4 to move and thus adjust the fecal passageway. FIG. 699B shows an embodiment identical to that of FIG. 699A, except that a reversing device in the form of an electric switch 14 also is implanted in the patient for reversing the restriction device 4. The wireless remote control of the external energy transmission means 10 transmits a wireless signal that carries energy and the implanted energy transforming means 6 transforms the wireless energy into a current for operating the switch 14. When the polarity of the current is shifted by the energy transforming means 6 the switch 14 reverses the function performed by the restriction device 4. FIG. 699C shows an embodiment identical to that of FIG. 699A, except that an operation device in the form of a motor 15 for operating the restriction device 4 also is implanted in the patient. The motor 15 is powered with energy from the energy transforming means 6, as the remote control of the external energy transmission means 10 transmits a wireless signal to the receiver of the energy transforming means 6.

FIG. 699D shows an embodiment identical to that of FIG. 699A, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to reduce the cross-sectional area of the fecal passageway, and hydraulic fluid is pumped by the motor/pump unit 18 back from the restriction device 4 to the reservoir 20 to enlarge the cross-sectional area. The implanted energy transforming means unit 6 transforms wireless energy into a current, for example a polarized current, for powering the motor/pump unit 18 via an electric power supply line 24.

FIG. 699E shows an embodiment comprising the external energy transmission means 10 with its wireless remote control, the restriction device 4, in this case hydraulically operated, and the implanted energy transforming means 6, and further comprising an implanted hydraulic fluid reservoir 30, an implanted motor/pump unit 32 and an implanted reversing device in the form of a hydraulic valve shifting device 34. The motor of the motor/pump unit 32 is an electric motor. In response to a control signal from the wireless remote control of the external energy transmission means 10, the implanted energy transforming means 6 powers the motor/pump unit 32 with energy from the energy carried by the control signal, whereby the motor/pump unit 32 distributes hydraulic fluid between the reservoir 30 and the restriction device 4. The remote control of the energy transmission means 10 controls the shifting device 34 to shift the hydraulic fluid flow direction between one direction in which the fluid is pumped by the motor/pump unit 32 from the reservoir 30 to the restriction device 4 to reduce the cross-sectional area of the fecal passageway, and another opposite direction in which the fluid is pumped by the motor/pump unit 32 back from the restriction device 4 to the reservoir 30 to enlarge the cross-sectional area. FIG. 699F shows an embodiment identical to that of FIG. 699A, except that a control unit 36 controlled by the wireless remote control of the external energy transmission means 10, an accumulator 38 and a capacitor 40 also are implanted in the patient. The control unit 36 stores electric energy received from the energy transforming means 6 in the accumulator 38, which supplies energy to the restriction device 4. In response to a control signal from the wireless remote control of the energy transmission means 10, the control unit 6 either releases electric energy from the accumulator 38 and transfers the released energy via power lines 42 and 44, or directly transfers electric energy from the energy transforming means 6 via a power line 46, the capacitor 40, which stabilizes the electric current, a power line 48 and the power line 44, for the operation of the restriction device 4. In accordance with one alternative, the capacitor 40 in the embodiment of FIG. 699F may be omitted. In accordance with another alternative, the accumulator 38 in this embodiment may be omitted.

FIG. 699G shows an embodiment identical to that of FIG. 699A, except that a battery 50 for supplying energy for the operation of the restriction device 4 and an electric switch 52 for switching the operation of the restriction device 4 also are implanted in the patient. The switch 52 is operated by the energy supplied by the energy transforming means 6 to switch from an off mode, in which the battery 50 is not in use, to an on mode, in which the battery 50 supplies energy for the operation of the restriction device 4.

FIG. 699H shows an embodiment identical to that of FIG. 699G, except that a control unit 36 controllable by the wireless remote control of the external energy transmission means 10 also is implanted in the patient. In this case, the switch 52 is operated by the energy supplied by the energy transforming means 6 to switch from an off mode, in which the wireless remote control is prevented from controlling the control unit 36 and the battery is not in use, to a standby mode, in which the remote control is permitted to control the control unit 36 to release electric energy from the battery 50 for the operation of the restriction device 4. FIG. 699I shows an embodiment identical to that of FIG. 699H, except that an accumulator 38 is substituted for the battery 50 and the implanted components are interconnected differently. In this case, the accumulator 38 stores energy from the energy transforming means 6. In response to a control signal from the wireless remote control of the external energy transmission means 10, the implanted control unit 36 controls the switch 52 to switch from an off mode, in which the accumulator 38 is not in use, to an on mode, in which the accumulator 38 supplies energy for the operation of the restriction device 4.

FIG. 699J shows an embodiment identical to that of FIG. 699I, except that a battery 50 also is implanted in the patient and the implanted components are interconnected differently. In response to a control signal from the wireless remote control of the external energy transmission means 10, the implanted control unit 36 controls the accumulator 38 to deliver energy for operating the switch 52 to switch from an off mode, in which the battery 50 is not in use, to an on mode, in which the battery 50 supplies electric energy for the operation of the restriction device 4. Alternatively, the switch 52 may be operated by energy supplied by the accumulator 38 to switch from an off mode, in which the wireless remote control is prevented from controlling the battery 50 to supply electric energy and is not in use, to a standby mode, in which the wireless remote control is permitted to control the battery 50 to supply electric energy for the operation of the restriction device 4.

FIG. 699K shows an embodiment identical to that of FIG. 699G, except that a motor 15, a mechanical reversing device in the form of a gearbox 54 and a control unit 36 for controlling the gearbox 54 also are implanted in the patient. The implanted control unit 36 controls the gearbox 54 to reverse the function performed by the restriction device 4 (mechanically operated).

FIG. 699L shows an embodiment identical to that of FIG. 699J except that the implanted components are interconnected differently. Thus, in this case the control unit 36 is powered by the battery 50 when the accumulator 38, suitably a capacitor, activates the switch 52 to switch to an on mode. When the switch 52 is in its on mode the control unit 36 is permitted to control the battery 50 to supply, or not supply, energy for the operation of the restriction device 4.

FIG. 699M schematically shows conceivable combinations of implanted components of the apparatus for achieving various communication options. Basically, there are the implanted restriction device 4, control unit 36 and motor/pump unit 18, and the external energy transmission means 10 including the external wireless remote control. As already described above the wireless remote control transmits a control signal which is received by the implanted control unit 36, which in turn controls the various implanted components of the apparatus. A sensor 56 may be implanted in the patient for sensing a physical parameter of the patient, such as the pressure in the fecal passageway. The implanted control unit 36, or alternatively the external wireless remote control of the energy transmission means 10, may control the restriction device 4 in response to signals from the sensor 56. A transceiver may be combined with the sensor 56 for sending information on the sensed physical parameter to the external wireless remote control. The wireless remote control may comprise a signal transmitter or transceiver and the implanted control unit 36 may comprise a signal receiver or transceiver. Alternatively, the wireless remote control may comprise a signal receiver or transceiver and the implanted control unit 36 may comprise a signal transmitter or transceiver. The above transceivers, transmitters and receivers may be used for sending information or data related to the restriction device 4 from inside the patient's body to the outside thereof.

Where the motor/pump unit 18 and battery 50 for powering the motor/pump unit 18 are implanted, the battery 50 may be equipped with a transceiver for sending information on the condition of the battery 50. Those skilled in the art will realize that the above various embodiments according to FIGS. 699A-699M could be combined in many different ways. For example, the energy operated switch 14 could be incorporated in any of the embodiments of FIGS. 699C, 699F-L, the hydraulic shifting device 34 could be incorporated in the embodiment of FIG. 699D, and the gearbox 54 could be incorporated in the embodiment of FIG. 699C.

Figure N shows an energy transforming means in the form of an electrical junction element 58 for use in any of the above embodiments according to FIGS. 699A-699M. The element 58 is a flat p-n junction element comprising a p-type semiconductor layer 60 and an n-type semiconductor layer 62 sandwiched together. A light bulb 64 is electrically connected to opposite sides of the element 58 to illustrate how the generated current is obtained.

The output of current from such a p-n junction element 58 is correlated to the temperature. See the formula below.

$$I = 10 ( exp ( qV / kT ) - 1 )$$

I is the external current flow,
I0 is the reverse saturation current, q is the fundamental electronic charge of 1.602×10-19 coulombs,
V is the applied voltage, k is the Boltzmann constant, and
T is the absolute temperature.

Under large negative applied voltage (reverse bias), the exponential term becomes negligible compared to 1.0, and I is approximately −10. 10 is strongly dependent on the temperature of the junction and hence on the intrinsic-carrier concentration. 10 is larger for materials with smaller bandgaps than for those with larger bandgaps. The rectifier action of the diode—that is, its restriction of current flow to only one direction—is in this particular embodiment the key to the operation of the p-n junction element 58.

An alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers such as but not limited to cadmium telluride, copper-indium-diselenide and silicon. It is also possible to use a multilayer structure with several layers of p and n-type materials to improve efficiency.

The electric energy generated by the p-n junction element 58 could be of the same type as generated by solar cells, in which the negative and positive fields create a direct current. Alternatively, the negative and positive semiconductor layers may change polarity following the transmitted waves, thereby generating an alternating current.

The p-n junction element 58 is designed to make it suited for implantation. Thus, all the external surfaces of the element 58 in contact with the human body are made of a biocompatible material. The p-n junction semiconductors are designed to operate optimally at a body temperature of 37° C. because the current output, which should be more than 1 μA, is significantly depending on temperature as shown above. Since both the skin and subcutis absorb energy, the relation between the sensitivity or working area of the element 58 and the intensity or strength of the wireless energy transmission is considered. The p-n junction element 58 preferably is designed flat and small. Alternatively, if the element 58 is made in larger sizes it should be flexible, in order to adapt to the patient's body movements. The volume of the element 58 should be kept less than 2000 cm3. FIG. 699O generally illustrates how any of the above-described embodiments of the anal incontinence treatment apparatus may be implanted in a patient. Thus, a restriction device 4 implanted in a patient engages the colon 66 to form an artificial sphincter around the fecal passageway in the colon. An implanted operation device 68, which may also be referred to as an adjustment device, such as an electric motor or a motor/pump assembly, operates the restriction device 4 through a transmission member 70, such as a mechanical transmission cord or a fluid tube. An energy transforming means in the form of an element 6 having a positive region and a negative region, as described above in more detail, is placed underneath the skin of the patient. Wireless energy carried by a signal transmitted by a wireless remote control of an external energy transmission means 10 at least partly penetrates the patient's skin and hits the element 6. The energy thus hitting the element 6 is transformed into energy of a different form that is suited for powering the operation device 68. For example, where the operation device 68 is an electric motor the element 6 comprises an electric p-n junction element that transforms the wireless energy into an electric current for powering the electric motor. Where the operation device 68 comprises a pump, the element 6 may transform the wireless energy into kinetic energy for powering the pump. The transformed energy may be utilized for directly operating the restriction device 4 or, where the restriction device 4 is electrically operated, for storage in a capacitor and/or an accumulator for later or parallel use. Preferably (but not necessarily) the element 6 is controlled by a microprocessor. The wireless remote control of the external energy transmission means 10 is used to control the utilization of the transmitted energy and any function or command to/from the implanted restriction device 4.

### W: Anal Incontinence Treatment 2

FIG. 699A schematically shows a most simple embodiment of the anal incontinence disease apparatus having some parts implanted in a patient and other parts located outside the patient's body. Thus, in FIG. 699A all parts placed to the right of the patient's skin 2 are implanted and all parts placed to the left of the skin 2 are located outside the patient's body.

The apparatus of FIG. 699A comprises an implanted operable restriction device 4, which engages the patient's colon (or alternatively engages the rectum) to form a restricted fecal passageway in the colon. The restriction device 4 is capable of performing a reversible function, i.e. to enlarge and reduce the cross-sectional area of the fecal passageway, whereby the restriction device 4 works as an artificial sphincter. An implanted energy transforming device 6 is adapted to supply energy consuming components of the restriction device 4 with energy via a power supply line 12. An external energy transmission device 10 includes a wireless remote control for transmitting a wireless signal, which is received by a signal receiver incorporated in the implanted energy transforming device 6. The implanted energy transforming device 6 transforms energy from the signal into electric energy that is supplied via the power supply line 12.

FIG. 699B shows an embodiment identical to that of FIG. 699A, except that a reversing device in the form of an electric switch 14 energy also is implanted in the patient for reversing the restriction device 4. The wireless remote control of the external energy transmission device 10 transmits a wireless signal that carries energy and the implanted energy transforming device 6 transforms the wireless energy into a current for operating the switch 14. When the polarity of the current is shifted by the energy transforming device 6 the switch 14 reverses the function performed by the restriction device 4.

FIG. 699C shows an embodiment identical to that of FIG. 699A, except that an operation device in the form of a motor 15 for operating the restriction device 4 also is implanted in the patient. The motor 15 is powered with energy from the energy transforming device 6, as the remote control of the external energy transmission device 10 transmits a wireless signal to the receiver of the energy transforming device 6.

FIG. 699D shows an embodiment identical to that of FIG. 699A, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to reduce the cross-sectional area of the fecal passageway, and hydraulic fluid is pumped by the motor/pump unit 18 back from the restriction device 4 to the reservoir 20 to enlarge the cross-sectional area. The implanted energy transforming device unit 6 transforms wireless energy into a current, for example a current, for powering the motor/pump unit 18 via an electric power supply line 24.

FIG. 699E shows an embodiment comprising the external energy transmission device 10 with its wireless remote control, the restriction device 4, in this case hydraulically operated, and the implanted energy transforming device 6, and further comprising an implanted hydraulic fluid reservoir 30, an implanted motor/pump unit 32 and an implanted reversing device in the form of a hydraulic valve shifting device 34. The motor of the motor/pump unit 32 is an electric motor. In response to a control signal from the wireless remote control of the external energy transmission device 10, the implanted energy transforming device 6 powers the motor/pump unit 32 with energy from the energy carried by the control signal, whereby the motor/pump unit 32 distributes hydraulic fluid between the reservoir 30 and the restriction device 4. The remote control of the energy transmission device 10 controls the shifting device 34 to shift the hydraulic fluid flow direction between one direction in which the fluid is pumped by the motor/pump unit 32 from the reservoir 30 to the restriction device 4 to reduce the cross-sectional area of the fecal passageway, and another opposite direction in which the fluid is pumped by the motor/pump unit 32 back from the restriction device 4 to the reservoir 30 to enlarge the cross-sectional area.

FIG. 699F shows an embodiment identical to that of FIG. 699A, except that a control unit 36 controlled by the wireless remote control of the external energy transmission device 10, an accumulator 38 and a capacitor 40 also are implanted in the patient. The control unit 36 stores electric energy received from the energy transforming device 6 in the accumulator 38, which supplies energy to the restriction device 4. In response to a control signal from the wireless remote control of the energy transmission device 10, the control unit 6 either releases electric energy from the accumulator 38 and transfers the released energy via power lines 42 and 44, or directly transfers electric energy from the energy transforming device 6 via a power line 46, the capacitor 40, which stabilizes the electric current, a power line 48 and the power line 44, for the operation of the restriction device 4. In accordance with one alternative, the capacitor 40 in the embodiment of FIG. 699F may be omitted. In accordance with another alternative, the accumulator 38 in this embodiment may be omitted.

FIG. 699G shows an embodiment identical to that of FIG. 699A, except that a battery 50 for supplying energy for the operation of the restriction device 4 and an electric switch 52 for switching the operation of the restriction device 4 also are implanted in the patient. The switch 52 is operated by the energy supplied by the energy transforming device 6 to switch from an off mode, in which the battery 50 is not in use, to an on mode, in which the battery 50 supplies energy for the operation of the restriction device 4.

FIG. 699H shows an embodiment identical to that of FIG. 699G, except that a control unit 36 controllable by the wireless remote control of the external energy transmission device 10 also is implanted in the patient. In this case, the switch 52 is operated by the energy supplied by the energy transforming device 6 to switch from an off mode, in which the wireless remote control is prevented from controlling the control unit 36 and the battery is not in use, to a standby mode, in which the remote control is permitted to control the control unit 36 to release electric energy from the battery 50 for the operation of the restriction device 4.

FIG. 699I shows an embodiment identical to that of FIG. 699H, except that an accumulator 38 is substituted for the battery 50 and the implanted components are interconnected differently. In this case, the accumulator 38 stores energy from the energy transforming device 6. In response to a control signal from the wireless remote control of the external energy transmission device 10, the implanted control unit 36 controls the switch 52 to switch from an off mode, in which the accumulator 38 is not in use, to an on mode, in which the accumulator 38 supplies energy for the operation of the restriction device 4.

FIG. 699J shows an embodiment identical to that of FIG. 699I, except that a battery 50 also is implanted in the patient and the implanted components are interconnected differently. In response to a control signal from the wireless remote control of the external energy transmission device 10, the implanted control unit 36 controls the accumulator 38 to deliver energy for operating the switch 52 to switch from an off mode, in which the battery 50 is not in use, to an on mode, in which the battery 50 supplies electric energy for the operation of the restriction device 4.

Alternatively, the switch 52 may be operated by energy supplied by the accumulator 38 to switch from an off mode, in which the wireless remote control is prevented from controlling the battery 50 to supply electric energy and is not in use, to a standby mode, in which the wireless remote control is permitted to control the battery 50 to supply electric energy for the operation of the restriction device 4.

FIG. 699K shows an embodiment identical to that of FIG. 699G, except that a motor 15, a mechanical reversing device in the form of a gearbox 54 and a control unit 36 for controlling the gearbox 54 also are implanted in the patient. The implanted control unit 36 controls the gearbox 54 to reverse the function performed by the restriction device 4 (mechanically operated).

FIG. 699L shows an embodiment identical to that of FIG. 699J except that the implanted components are interconnected differently. Thus, in this case the battery 50 powers the control unit 36 when the accumulator 38, suitably a capacitor, activates the switch 52 to switch to an on mode. When the switch 52 is in its on mode the control unit 36 is permitted to control the battery 50 to supply, or not supply, energy for the operation of the restriction device 4.

FIG. 699M schematically shows conceivable combinations of implanted components of the apparatus for achieving various communication options. Basically, there are the implanted restriction device 4, control unit 36 and motor/pump unit 18, and the external energy transmission device 10 including the external wireless remote control. As already described above the wireless remote control transmits a control signal which is received by the implanted control unit 36, which in turn controls the various implanted components of the apparatus.

A sensor 56 may be implanted in the patient for sensing a physical parameter of the patient, such as the pressure in the fecal passageway. The implanted control unit 36, or alternatively the external wireless remote control of the energy transmission device 10, may control the restriction device 4 in response to signals from the sensor 56. A transceiver may be combined with the sensor 56 for sending information on the sensed physical parameter to the external wireless remote control. The wireless remote control may comprise a signal transmitter or transceiver and the implanted control unit 36 may comprise a signal receiver or transceiver. Alternatively, the wireless remote control may comprise a signal receiver or transceiver and the implanted control unit 36 may comprise a signal transmitter or transceiver. The above transceivers, transmitters and receivers may be used for sending information or data related to the restriction device 4 from inside the patient's body to the outside thereof.

Where the motor/pump unit 18 and battery 50 for powering the motor/pump unit 18 are implanted, the battery 50 may be equipped with a transceiver for sending information on the condition of the battery 50. Those skilled in the art will realize that the above various embodiments according to FIGS. 699A-699M could be combined in many different ways. For example, the energy operated switch 14 could be incorporated in any of the embodiments of FIGS. 699C, 699F-L, the hydraulic shifting device 34 could be incorporated in the embodiment of FIG. 699D, and the gearbox 54 could be incorporated in the embodiment of FIG. 699C.

FIG. 699N shows an energy transforming device in the form of an electrical junction element 58 for use in any of the above embodiments according to FIGS. 699A-699M. The element 58 is a flat p-n junction element comprising a p-type semiconductor layer 60 and an n-type semiconductor layer 62 sandwiched together. A light bulb 64 is electrically connected to opposite sides of the element 58 to illustrate how the generated current is obtained. The output of current from such a p-n junction element 58 is correlated to the temperature. See the formula below.

$$I = 10 ( exp ( qV / kT ) - 1 )$$

where
I is the external current flow, 10 is the reverse saturation current, q is the fundamental electronic charge of 1.602×10-19 coulombs,
V is the applied voltage, k is the Boltzmann constant, and
T is the absolute temperature.

Under large negative applied voltage (reverse bias), the exponential term becomes negligible compared to 1.0, and I is approximately −10. 10 is strongly dependent on the temperature of the junction and hence on the intrinsic-carrier concentration. 10 is larger for materials with smaller bandgaps than for those with larger bandgaps. The rectifier action of the diode—that is, its restriction of current flow to only one direction—is in this particular embodiment the key to the operation of the p-n junction element 58.

An alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers such as but not limited to cadmium telluride, copper-indium-diselenide and silicon. It is also possible to use a multilayer structure with several layers of p and n-type materials to improve efficiency.

The electric energy generated by the p-n junction element 58 could be of the same type as generated by solar cells, in which the negative and positive fields create a direct current. Alternatively, the negative and positive semiconductor layers may change polarity following the transmitted waves, thereby generating an alternating current.

The p-n junction element 58 is designed to make it suited for implantation. Thus, all the external surfaces of the element 58 in contact with the human body are made of a biocompatible material. The p-n junction semiconductors are designed to operate optimally at a body temperature of 37° C. because the current output, which should be more than 1 μA, is significantly depending on temperature as shown above. Since both the skin and subcutis absorb energy, the relation between the sensitivity or working area of the element 58 and the intensity or strength of the wireless energy transmission is considered. The p-n junction element 58 preferably is designed flat and small. Alternatively, if the element 58 is made in larger sizes it should be flexible, in order to adapt to the patient's body movements. The volume of the element 58 should be kept less than 2000 cm3.

FIG. 699O generally illustrates how any of the above-described embodiments of the anal incontinence disease treatment apparatus may be implanted in a patient. Thus, a restriction device 4 implanted in a patient engages the colon 66 to form an artificial sphincter around the fecal passageway in the colon 66. An implanted operation device 68, such as an electric motor or a motor/pump assembly, operates the restriction device 4 through a transmission member 70, such as a mechanical transmission cord or a fluid tube. An energy transforming device in the form of an element 6 having a positive region and a negative region, as described above in more detail, is placed underneath the skin of the patient. Wireless energy carried by a signal transmitted by a wireless remote control of an external energy transmission device 10 at least partly penetrates the patient's skin and hits the element 6. The energy thus hitting the element 6 is transformed into energy of a different form that is suited for powering the operation device 68. For example, where the operation device 68 is an electric motor the element 6 comprises an electric p-n junction element that transforms the wireless energy into an electric current for powering the electric motor. Where the operation device 68 comprises a pump, the element 6 may transform the wireless energy into kinetic energy for powering the pump. The transformed energy may be utilized for directly operating the restriction device 4 or, where the restriction device 4 is electrically operated, for storage in a capacitor and/or an accumulator for later or parallel use. Preferably (but not necessarily) the element 6 is controlled by a microprocessor. The wireless remote control of the external energy transmission device 10 is used to control the utilization of the transmitted energy and any function or command to/from the implanted restriction device 4.

FIG. 699P shows the basic parts of a wireless remote control of the apparatus including an electric motor 128 for operating a restriction member, for example of the type illustrated in FIG. 699O. In this case, the remote control is based on the transmission of electromagnetic wave signals, often of high frequencies in the order of 100 kHz-1 gHz, through the skin 130 of the patient. In FIG. 699O, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134. An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either contract or enlarge the restriction device. The microprocessor starts a command by applying a high frequency signal on the antenna 132. After a short time, when the signal has energized the implanted parts of the control system, commands are sent to contract or enlarge the restriction device in predefined steps. The commands are sent as digital packets in the form illustrated below. The commands are sent continuously during a rather long time period (e.g. about 30 seconds or more). When a new contract or enlarge step is desired the Count byte is increased by one to allow the implanted control unit 138 to decode and understand that another step is demanded by the external control unit 136. If any part of the digital packet is erroneous, its content is simply ignored. Through a line 140, an implanted energizer unit 126 draws energy from the high frequency electromagnetic wave signals received by the receiving antenna 134. The energizer unit 126 stores the energy in an energy storage device, such as a large capacitor, powers the control unit 138 and powers the electric motor 128 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to either contract or enlarge the restriction device depending on the received command code. Alternatively, the energy stored in the energy storage device of the energizer unit may only be used for powering a switch, and the energy for powering the motor 128 may be obtained from another implanted energy source of relatively high capacity, for example a battery. In this case the switch is adapted to connect said battery to the control unit 138 in an on mode when said switch is powered by the energy storage device and to keep the battery disconnected from the control unit in a standby mode when the switch is not powered. With reference to FIG. 699Q, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178. The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### X: Hydraulic Anal Incontinence Treatment Device

FIGS. 700-1A-D is a block diagram of four different embodiments of the anal incontinence treatment apparatus according to the invention. FIG. 700-1A shows an elongated restriction member in the form of a band 2 forming a loop which defines a restriction opening. The band 2 provides a restricted fecal passageway in the rectum when applied around the latter. FIG. 700-1A further shows a separate reservoir 4, a one way pump 6 and an alternate valve 8. FIG. 700-1B shows the band 2 and a fluid supply reservoir 10. FIG. 700-1C shows the band 2, a two way pump 12 and the reservoir 4. FIG. 700-1D shows a servo system with a first closed system controlling a second system. The servo system comprises the fluid supply reservoir 10 and a servo reservoir 14. The servo reservoir 14 controls a larger adjustable reservoir 16 which in connection with the band 2 applied around the rectum varies the volume of a cavity in the band, which in turn varies the restricted fecal passageway in the rectum. Such a band 2 forming the restriction opening 3 is illustrated schematically in FIG. 700-8. The band 2 comprises an adjustment device having an expandable/contractable cavity 5 which is expanded or contracted by supplying hydraulic fluid (e.g. from reservoir 4, 6, 10, or 16), and the band 2 may be sutured in place, illustrated schematically at 7 in FIG. 700-8. FIGS. 700-2A-D are cross-sectional views of a pump mechanism adapted to pump fluid in both directions only by mechanically pushing a separate sealing wall portion 18 in one direction. FIG. 700-2A shows a piston 20 pushed forwards against a spring 22 towards the wall portion 18 and located in a pump housing 24 conducting fluid from a right upper fluid passage 26 of the housing 24 to a left fluid passage 28 of the housing 24. A main valve 30 is open and a nonreturn valve 32 is closed. FIG. 700-2B illustrates the first pump movement in which the piston 20 has moved forwards and reaches the wall portion 18. FIG. 700-2C illustrates how the piston 20 moves backwards by the action of the spring 22. The main valve 30 is now closed and the nonreturn valve 32 is open for fluid from the right upper passage 26. FIG. 700-1D illustrates how the piston 20 is moved further downwards from its position according to FIG. 700-2B while pushing the wall portion 18 downwardly against a second spring 34 that is stronger than spring 22, whereby fluid escapes from a right lower fluid passage 36. When moving the piston 20 backwardly from the position according to FIG. 700-2D, fluid enters the left fluid passage 28 and a valve 38 in the lower right fluid passage 36 closes.

FIG. 700-3 is a cross-sectional view of a reservoir 40 defining a chamber 42, the size of which is variable by an operation device comprising a remote controlled electric motor 44, in accordance with FIG. 700-1B or 700-1D. The reservoir 40 and the motor 44 are placed in a housing 46. The chamber 42 is varied by moving a large wall 48. The wall 48 is secured to a nut 50, which is threaded on a rotatable spindle 52. The spindle 52 is rotated by the motor 44 via an angular gearing, which comprises two conical gear wheels 54 and 56 in mesh with each other. The motor 44 is powered by a battery 58 placed in the housing 46. An signal receiver 60 for controlling the motor 44 is also placed in the housing 46. Alternatively, the battery 58 and the signal receiver 60 may be mounted in a separate place. The motor 44 may also be powered by energy transferred from transmitted signals.

FIG. 700-4 is a cross-sectional view of a reservoir 62 defining a chamber 64, the size of which is variable and is controlled by manual manipulation. A gable wall portion 66 of an open ended inner cylindrical housing 68 is adapted to be pushed downwards to fit in a desired locking groove 70 of a plurality of locking grooves 70 on the mantle wall of the cylindrical housing 68, to reduce the volume of the chamber 64. The inner cylindrical housing 68 is suspended by springs 72 and is telescopically applied on an outer cylindrical housing 74. When pushing the inner cylindrical housing 68 it moves downwards relative to the outer cylindrical housing 74 causing the gable wall portion 66 to release from the locking groove 70 and move upwards relative to the inner cylindrical housing 68. When the inner housing 68 is moved upwardly by the action of the springs 72 the volume of the chamber 64 is increased.

FIGS. 700-5A and 700-5B show a servo means comprising a main ring-shaped fluid reservoir 76 defining a chamber 78, the size of which is variable. Centrally positioned in the main ring-shaped reservoir 76 there is a servo fluid reservoir 80 defining a chamber 82, the size of which is variable. The chamber 82 of the servo reservoir 80 is substantially smaller than the chamber 78 of the main reservoir 76. The two reservoirs 76 and 80 are situated between two opposite separate walls 84 and 86, and are secured thereto. When changing the amount of fluid in the servo reservoir 80, the two opposite walls 84, 86 are moved towards or away from each other, whereby the volume of the chamber 78 of the main reservoir 76 is changed. FIG. 700-6 shows the basic parts of a remote control system of the apparatus including the electric motor 44 of the embodiment shown in FIG. 700-3. In this case, the remote control system is based on the transmission of electromagnetic wave signals, often of high frequencies in the order of 100 kHz-1 gHz, through the skin 130 of the patient. In FIG. 700-6, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134. An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either increase or decrease the size of the restriction opening defined by the loop of the restriction member 2. The microprocessor starts a command by applying a high frequency signal on the antenna 132. After a short time, when the signal has energized the implanted parts of the control system, commands are sent to increase or decrease the size of the restriction opening of the restriction member 2 in predefined steps. The commands are sent as digital packets in the form illustrated below.

Start Command, Count, Checks pattern, 8 d, 8 bits 8 bits urn, 8 bits

The commands are sent continuously during a rather long time period (e.g. about 30 seconds or more). When a new increase or decrease step is desired the Count byte is increased by one to allow the implanted control unit 138 to decode and understand that another step is demanded by the external control unit 136. If any part of the digital packet is erroneous, its content is simply ignored.

Through a line 140, an implanted energizer unit 126 draws energy from the high frequency electromagnetic wave signal received by the receiving antenna 134. The energizer unit 126 stores the energy in a power supply, such as a large capacitor, powers the control unit 138 and powers the electric motor 44 via a line 142.

The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 44 to either increase or decrease the size of the restriction opening of the restriction member 2 depending on the received command code.

Alternatively, the energy stored in the power supply of the energizer unit may only be used for powering a switch, and the energy for powering the motor 44 may be obtained from another implanted power source of relatively high capacity, for example a battery. In this case the switch is adapted to connect the battery to the control unit 138 in an on mode when the switch is powered by the power supply and to keep the battery disconnected from the control unit in a standby mode when the switch is unpowered.

With reference to FIG. 700-7, the remote control system schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the anal incontinence apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148.

The implanted signal receiving antenna coil 134 forms together with a capacitor 154 a resonant circuit that is tuned to. the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation.

A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 44 via an H-bridge 178 comprising transistors 180, 182, 184 and 186. The motor 44 can be driven in two opposite directions by the H-bridge 178.

The microprocessor 176 also monitors the amount of stored energy in the storage capacitor 158. Before sending signals to activate the motor 44, the microprocessor 176 checks whether the energy stored in the storage capacitor 158 is enough. If the stored energy is not enough to perform the requested operation, the microprocessor 176 waits for the received signals to charge the storage capacitor 158 before activating the motor 44.

FIGS. 700-9A and 700-9B show an embodiment of the apparatus comprising a restriction device 202 having an elongated flexible restriction member 204, such as a belt, a cord or the like. The flexible member 204 extends in a loop around the rectum. (Alternatively, the flexible member 204 may comprise two separate parts on opposite sides of the rectum.) One portion 204A of member 204 is attached to a frame 208 and another portion 204B of member 204 opposite portion 204A in the loop of the flexible member 204 is connected to an adjustment device 210, which is fixed to the frame 208. The adjustment device 210 pulls the flexible member 204 away from portion 204A to squeeze the rectum between two opposite lengths of the flexible member 204 to thereby restrict the fecal passageway, see FIG. 700-9A, and releases the rectum from the flexible member 204 to thereby increase the fecal passageway, see FIG. 700-9B.

FIGS. 700-10A and 700-10B show an embodiment of the apparatus comprising a restriction device 212 having two plate or bar elements 214 on opposite sides of the rectum 206. An adjustment device 216 moves the elements 212 in parallel towards each other to squeeze the rectum 206 between the elements 212 to thereby restrict the fecal passageway, see FIG. 700-10A, and moves the elements 212 away from each other to increase the fecal passageway, see FIG. 700-10B.

FIG. 700-11 shows an embodiment of the apparatus comprising a restriction device 218 having two articulated clamping elements 220 positioned on opposite sides of the rectum 206. An adjustment device 222 moves the clamping elements 220 toward each other to clamp the rectum 206 between the clamping elements 220 to thereby restrict the fecal passageway, and moves the clamping elements 220 away from each other to release the rectum 206 from the clamping elements 220 to thereby increase the fecal passageway. FIGS. 700-12A, 700-12B and 700-12C show an embodiment of the apparatus comprising a restriction device 224 having three bending members in the form of cylindrical rollers 226, 228 and 230 displaced relative one another in a row along the rectum 206 and positioned alternately on opposite sides of the rectum 206. (Alternatively, each roller 226, 228 and 230 may take the shape of an hour-glass.) An adjustment device 232 moves the two outer rollers 226, 230 laterally against the rectum 206 in one direction and the intermediate roller 228 against the rectum 206 in the opposite direction to bend the rectum to thereby restrict the fecal passageway, see FIG. 700-12B. To release the rectum from the rollers 226-230, the adjustment device 232 moves the rollers 226-230 away from the rectum 206, see FIG. 700-12C.

FIGS. 700-13A through 700-17B schematically illustrates modifications of the above embodiment according to FIGS. 700-12A-12C. Thus, FIGS. 700-13A and 700-13B show an embodiment similar to that of FIGS. 700-12A-700-12C except that the bending members are oval and not rotatable.

FIGS. 700-14A and 700-14B show an embodiment similar to that of FIGS. 700-13A and 700-13B except that the oval bending members are rotatable to squeeze the rectum, see FIG. 700-14B, and to release the rectum, see FIG. 700-14A.

FIGS. 700-15A and 700-15B show an embodiment similar to that of FIGS. 700-12A-700-12C except that the intermediate roller has a changeable diameter to squeeze the rectum, see FIG. 15B, and to release the rectum, see FIG. 700-15A. FIGS. 700-16A and 700-16B show an embodiment similar to that of FIGS. 700-10A-700-10C except that the elements are replaced by two cylindrical rollers positioned on opposite sides of the rectum. Finally, FIGS. 700-17A and 700-17B show an embodiment substantially similar to that of FIGS. 700-16A and 700-16B except that the restriction device is turned 90° to form a S-shaped curvature of the rectum.

FIG. 700-18 shows an example of a hydraulic restriction device 234 for use in accordance with the invention. The restriction device 234 comprises an elongated restriction member 236 having an inflatable cavity 238. A tube 240 connects the cavity 238 to a hydraulic fluid reservoir, not shown. The restriction member 23-6 may be wrapped around the rectum.

FIG. 19 schematically illustrates how any of the above-described embodiments of the anal incontinence treatment apparatus can be implanted in a patient. Thus, an implanted adjustable hydraulic restriction device 234 extends around the rectum 242 to be capable of squeezing the rectum 242. An adjustment device in the form of an inflatable cavity in the restriction device 234 is adapted to adjust the restriction device 234 so that the fecal passageway is restricted. An implanted assembly 246 includes a hydraulic fluid reservoir and an operation device (which may include a pump) for distributing hydraulic fluid between the reservoir and the inflatable/contractible cavity of the restriction device 234 via a fluid conduit 240. A wireless remote control of the apparatus comprises an external signal transmitter 248, which may comprise a hand-held unit, and an implanted signal receiver, which is incorporated in the implanted assembly 246, includes a control unit for controlling the restriction device 234 in response to a control signal from the external transmitter. The signal receiver of the assembly 246 further includes an energizer unit which transforms energy from the control signal transmitted by the external transmitter into electric energy for energy consuming implanted components of the apparatus. A pressure sensor 250 is implanted for sensing the pressure on the restriction device 234. The control unit of the signal receiver of the implanted assembly 246 controls the restriction device 234 to release the restriction device 234 in response to the pressure sensor 250—sensing an abnormal high pressure. There are a number of conceivable alternative embodiments that give the same result as the above-described embodiments. For example, the microprocessor of the external and implanted, respectively, control units may be replaced by discrete components. The power amplifier of the external control unit may be omitted if the signals generated by the signal generator are strong enough. Therefore, the invention is to be accorded the broadest interpretation of the appended claims to encompass all equivalent structures and assemblies.

### Y: Anal Incontinence Treatment with Controlled Wireless Energy Supply

FIG. 701-1 schematically shows an embodiment of the anal incontinence treatment apparatus having some parts implanted in a patient and other parts located outside the patient's body. Thus, in FIG. 701-1 all parts placed to the right of the patient's skin 2 are implanted and all parts placed to the left of the skin 2 are located outside the patient's body. The apparatus of FIG. 701-1 comprises an implanted operable restriction device 4, which engages the patient's colon (or alternatively the rectum or anus) to form a restricted fecal passageway in the colon. The restriction device 4 is capable of performing a reversible function, i.e. to open and close the fecal passageway. An implanted control unit 6 controls the restriction device 4 via a control line 8 to form an adequate restriction of the fecal passageway. An external control unit 10 includes an external source of energy and a wireless remote control transmitting a control signal generated by the external source of energy. The control signal is received by a signal receiver incorporated in the implanted control unit 6, whereby the control unit 6 controls the implanted restriction device 4 in response to the control signal. The implanted control unit 6 also uses energy from the control signal for operating the restriction device 4 via a power supply line 12.

FIG. 701-2 shows an embodiment identical to that of FIG. 701-1, except that a reversing device in the form of a switch 14 operable by energy also is implanted in the patient for reversing the restriction device 4. The control unit 6 uses the switch 14 to reverse the function performed by the restriction device 4. More precisely, the external control unit 10 releases energy carried by a wireless signal and the implanted control unit 6 transforms the wireless energy into a current for operating the switch 14. When the control unit 6 shifts the polarity of the current the switch 14 reverses the function performed by the restriction device 4.

FIG. 701-3 shows an embodiment identical to that of FIG. 701-1, except that an operation device in the form of a motor 16 also is implanted in the patient. The implanted control unit 6 powers the motor 16 with wireless energy released from the external source of energy of the external control unit 10. The implanted control unit 6 controls the operation of the motor 16 in response to a control signal from the remote control of the external control unit 10. FIG. 701-4 shows an embodiment identical to that of FIG. 701-1, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to restrict the fecal passageway, and hydraulic fluid is pumped by the motor/pump unit 18 back from the restriction device 4 to the reservoir 20 to enlarge the fecal passageway. The external control unit 10 releases energy carried by a wireless signal and the implanted control unit 6 transforms the wireless energy into a current, for example a current, for powering the motor/pump unit 18 via an electric power supply line 24. The implanted control unit 6 controls the motor/pump unit 16 and the restriction device 4 via control lines 26 and 27. FIG. 701-5 shows an embodiment comprising the restriction device 4, hydraulically operated, and the implanted control unit 6, and further comprising a hydraulic fluid reservoir 230, a motor/pump unit 232 and a reversing device in the form of a hydraulic valve shifting device 234, all of which are implanted in the patient. The motor of the motor/pump unit 232 is an electric motor. FIG. 701-6 shows an embodiment identical to that of FIG. 701-1, except that an accumulator 28 also is implanted in the patient. The control unit 6 stores energy received from the external control unit 10 in the accumulator 28. In response to a control signal from the external control unit 10 the implanted control unit 6 releases energy from the accumulator 28 via a power line 30 for the operation of the restriction device 4.

FIG. 701-7 shows an embodiment comprising the restriction device 4, hydraulically operated, and the implanted control unit 6, and further comprising a source of energy in the form of a battery 32, a hydraulic fluid reservoir 34, a motor/pump unit 36 and a reversing device in the form of a hydraulic valve shifting device 38, all of which are implanted in the patient. The motor of the motor/pump unit 36 is an electric motor. An external control unit 40 includes a wireless remote control transmitting a control signal which is received by the signal receiver incorporated in the implanted control unit 6.

In response to a control signal from the external control unit 40 the implanted control unit 6 powers the motor/pump unit 36 with energy from the battery 32, whereby the motor/pump unit 36 distributes hydraulic fluid between the reservoir 34 and the restriction device 4. The control unit 6 controls the shifting device 38 to shift the hydraulic fluid flow direction between one direction in which the fluid is pumped by the motor/pump unit 36 from the reservoir 34 to the restriction device 4 to restrict the fecal passageway, and another opposite direction in which the fluid is pumped by the motor/pump unit 36 back from the restriction device 4 to the reservoir 34 to enlarge the fecal passageway.

FIG. 701-8 shows an embodiment identical to that of FIG. 701-6, except that a battery 42 is substituted for the accumulator 28, the external control unit 40 of the embodiment of FIG. 701-5 is substituted for the external control unit 10 and an electric motor 44 is implanted in the patient for operating the restriction device 4. In response to a control signal from the external control unit 40 the implanted control unit 6 powers the motor 44 with energy from the battery 42, whereby the motor 44 operates the restriction device 4.

FIG. 701-9 shows an embodiment identical to that of FIG. 701-8, except that the motor/pump unit 36 of the embodiment of FIG. 701-7 is substituted for the motor 44 and a fluid reservoir 46 also is implanted in the patient. The reservoir 46 is via fluid conduits 48 and 50 connected to the motor/pump unit 36 and restriction device 4, which in this case is hydraulically operated. In response to a control signal from the external control unit 40, the implanted control unit 6 powers the electric motor of the motor/pump unit 36 with energy from the battery 42, whereby the motor/pump unit 36 distributes hydraulic fluid between the fluid reservoir 46 and the restriction device 4. FIG. 701-10 shows an embodiment identical to that of FIG. 701-8, except that a mechanical reversing device in the form of a gearbox 52 also is implanted in the patient. The implanted control unit 6 controls the gearbox 52 to reverse the function performed by the restriction device 4 (mechanically operated).

FIG. 701-11 shows an embodiment comprising the restriction device 4, the external control unit 10, an implanted source of energy 236 and an implanted switch 238. The switch 238 is operated by wireless energy released from the external source of energy of the external control unit 6 to switch between an off mode, in which the implanted source of energy 236 is not in use, and an on mode, in which the implanted source of energy 236 supplies energy for the operation of the restriction device 4.

FIG. 701-12 shows an embodiment identical to that of FIG. 701-11, except that also the control unit 6 is implanted, in order to receive a control signal from the wireless remote control of the external control unit 10. The switch 238 is operated by the wireless energy from the external source of energy 10 to switch between an off mode, in which the implanted source of energy 236 and the wireless remote control of the external control unit 10 are not in use, i.e. the control unit 6 is not capable of receiving the control signal, and a standby mode, in which the wireless remote control is permitted to control the internal source of energy 236, via the implanted control unit 6, to supply energy for the operation of the restriction device 4.

FIG. 701-13 shows an embodiment identical to that of FIG. 701-12, except that an energy transforming device for transforming the wireless energy into storable energy is incorporated in the implanted control unit 6 and that the implanted source of energy 236 is of a type that is capable of storing the storable energy. In this case, in response to a control signal from the external control unit 10, the implanted control unit 6 controls the switch 238 to switch from an off mode, in which the implanted source of energy 236 is not in use, to an on mode, in which the source of energy 36 supplies energy for the operation of the restriction device 4.

FIG. 701-14 shows an embodiment identical to that of FIG. 701-13, except that an energy storage device 240 also is implanted in the patient for storing the storable energy transformed from the wireless energy by the transforming device of the control unit 6. In this case, the implanted control unit 6 controls the energy storage device 240 to operate the switch 238 to switch between an off mode, in which the implanted source of energy 236 is not in use, and an on mode, in which the implanted source of energy 236 supplies energy for the operation of the restriction device 4. FIG. 701-15 shows an embodiment identical to that of FIG. 701-13, except that a motor 242 and a mechanical reversing device in the form of a gearbox 244 also are implanted in the patient. The implanted control unit 6 controls the gearbox 244 to reverse the function performed by the restriction device 4 (mechanically operated), i.e. enlarging and restricting the fecal passageway.

FIG. 701-16 schematically shows conceivable combinations of implanted components of the apparatus for achieving various communication possibilities. Basically, there are the implanted restriction device 4, the implanted control unit 6 and the external control unit 10 including the external source of energy and the wireless remote control. As already described above the remote control transmits a control signal generated by the external source of energy, and the control signal is received by a signal receiver incorporated in the implanted control unit 6, whereby the control unit 6 controls the implanted restriction device 4 in response to the control signal. A sensor 54 may be implanted in the patient for sensing a physical parameter of the patient, such as the pressure in the stomach. The control unit 6, or alternatively the external control unit 10, may control the restriction device 4 in response to signals from the sensor 54. A transceiver may be combined with the sensor 54 for sending information on the sensed physical parameter to the external control unit 10. The wireless remote control of the external control unit 10 may comprise a signal transmitter or transceiver and the implanted control unit 6 may comprise a signal receiver or transceiver. Alternatively, the wireless remote control of the external control unit 10 may comprise a signal receiver or transceiver and the implanted control unit 6 may comprise a signal transmitter or transceiver. The above transceivers, transmitters and receivers may be used for sending information or data related to the restriction device from inside the patient's body to the outside thereof.

The motor 44 may be implanted for operating the restriction device 4 and also the battery 32 may be implanted for powering the motor 44. The battery 32 may be equipped with a transceiver for sending information on the charge condition of the battery.

Those skilled in the art will realize that the above various embodiments according to FIGS. 701-1-701-15 could be combined in many different ways. For example, the energy operated switch 14 could be incorporated in any of the embodiments of FIGS. 701-4, 701-6, 701-8-701-10. The hydraulic shifting device 38 could be incorporated in any of the embodiments of FIGS. 701-4 and 701-9. The gearbox 52 could be incorporated in any of the embodiments of FIGS. 701-1, 701-6 and 701-8.

FIG. 701-17 illustrates how any of the above-described embodiments of the apparatus may be implanted in a patient. Thus, an assembly of the apparatus implanted in the patient comprises a restriction device 56 engaging the urethra 58, an operation device 60 for operating the restriction device 56 and an internal control unit 62, which includes a signal receiver, for controlling the operation device 60. An external control unit 64 includes a signal transmitter for transmitting a wireless control signal to the signal receiver of the implanted control unit 62. The implanted control unit 62 is capable of transforming signal energy from the control signal into electric energy for powering the operation device 60 and for energizing energy consuming implanted components of the apparatus.

FIG. 701-18 shows the basic parts of a wireless remote control of the apparatus including an electric motor 128 for operating a restriction member, for example of the type illustrated in FIG. 701-17. In this case, the remote control is based on the transmission of electromagnetic wave signals, often of high frequencies in the order of 100 kHz-1 gHz, through the skin 130 of the patient. In FIG. 701-18, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same restriction as the coil of the receiving antenna 134 but wound with a thick wire that can handle the larger currents that is necessary. The coil of the transmitting antenna 132 is tuned to the same specific high frequency as the coil of the receiving antenna 134.

An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either contract or enlarge the restriction device. The microprocessor starts a command by applying a high frequency signal on the antenna 132. After a short time, when the signal has energized the implanted parts of the control system, commands are sent to contract or enlarge the restriction device in predefined steps. The commands are sent as digital packets in the form illustrated below.
Start pattern, Command, Count, Checksum, 8 bits 8 bits 8 bits 8 bits

The commands are sent continuously during a rather long time period (e.g. about 30 seconds or more). When a new contract or enlarge step is desired the Count byte is increased by one to allow the implanted control unit 138 to decode and understand that another step is demanded by the external control unit 136. If any part of the digital packet is erroneous, its content is simply ignored. Through a line 140, an implanted energizer unit 126 draws energy from the high frequency electromagnetic wave signals received by the receiving antenna 134. The energizer unit 126 stores the energy in a power supply, such as a large capacitor, powers the control unit 138 and powers the electric motor 128 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to either contract or enlarge the restriction device depending on the received command code. Alternatively, the energy stored in the power supply of the energizer unit may only be used for powering a switch, and the energy for powering the motor 128 may be obtained from another implanted power source of relatively high capacity, for example a battery. In this case the switch is adapted to connect said battery to the control unit 138 in an on mode when said switch is powered by said power supply and to keep said battery disconnected from the control unit in a standby mode when said switch is unpowered. With reference to FIG. 701-19, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178. The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### Z: Impotence Treatment

FIG. 702-1 schematically shows a very simple embodiment of the impotence apparatus having some parts implanted in a patient and other parts located outside the patient's body. Thus, in FIG. 702-1 all parts placed to the right of the patient's skin 2 are implanted and all parts placed to the left of the skin 2 are located outside the patient's body.

The apparatus of FIG. 702-1 comprises an implanted operable restriction device 4, which engages penile tissue or the prolongation thereof to form a restrictable passageway for blood flow leaving the penis. The restriction device 4 is capable of performing a reversible function, i.e. to enlarge and restrict the passageway, so that the restriction device 4 works as an artificial sphincter. The implanted energy transforming means 6 is adapted to supply energy consuming components of the restriction device 4 with energy via a power supply line 12. The external energy transmission means 10 includes a wireless remote control transmitting a wireless signal which is received by a signal receiver incorporated in the implanted energy transforming means 6. The implanted energy transforming means 6 transforms energy from the signal into electric energy which is supplied via the power supply line 12 to the restriction device 4, which energy causes portions of the device 4 to move and thus adjust the opening.

FIG. 702-2 shows an embodiment identical to that of FIG. 702-1, except that a reversing device in the form of an electric switch 14 also is implanted in the patient for reversing the restriction device 4. The wireless remote control of the external energy transmission means 10 transmits a wireless signal that carries energy and the implanted energy transforming means 6 transforms the wireless energy into a current for operating the switch 14. When the polarity of the current is shifted by the energy transforming means 6 the switch 14 reverses the function performed by the restriction device 4.

FIG. 702-3 shows an embodiment identical to that of FIG. 702-1, except that the operation device in the form of a motor 15 for operating the restriction device 4 also is implanted in the patient. The motor 15 is powered with energy from the energy transforming means 6, as the remote control of the external energy transmission means 10 transmits a wireless signal to the receiver of the energy transforming means 6.

FIG. 702-4 shows an embodiment identical to that of FIG. 702-1, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to restrict the passageway, and hydraulic fluid is pumped by the motor/pump unit 18 back from the restriction device 4 to the reservoir to enlarge the cross-sectional area. The implanted energy transforming means 6 transforms wireless energy into a current, for powering the motor/pump unit 18 via the electric power supply line 24. FIG. 702-5 shows an embodiment comprising the external energy transmission means 10 with its wireless remote control, the restriction device 4, in this case hydraulically operated, and the implanted energy transforming means 6, and further comprising an implanted hydraulic fluid reservoir 30, an implanted motor/pump unit 32 and an implanted reversing device in the form of a hydraulic valve shifting device 34. The motor of the motor/pump unit 32 is the electric motor. In response to a control signal from the wireless remote control of the external energy transmission means 10, the implanted energy transforming means 6 powers the motor/pump unit 32 with energy from the energy carried by the control signal, whereby the motor/pump unit 32 distributes hydraulic fluid between the reservoir 30 and the restriction device 4. The remote control of the energy transmission means 10 controls the shifting device 34 to shift the hydraulic fluid flow direction between one direction in which the fluid is pumped by the motor/pump unit 32 from the reservoir 30 to the restriction device 4 to reduce the passageway, and another opposite direction in which the fluid is pumped by the motor/pump unit 32 back from the restriction device 4 to the reservoir 30 to enlarge the cross-sectional area.

FIG. 702-6 shows an embodiment identical to that of FIG. 702-1, except that a control unit 36 controlled by the wireless remote control of the external energy transmission means 10, an accumulator 38 and a capacitor 40 also are implanted in the patient. The control unit 36 stores electric energy received from the energy transforming means 6 in the accumulator 38, which supplies energy to the restriction device 4. In response to a control signal from the wireless remote control of the energy transmission means 10, the control unit 6 either releases electric energy from the accumulator 38 and transfers the released energy via power lines 42 and 44, or directly transfers electric energy from the energy transforming means 6 via a power line 46, the capacitor 40, which stabilizes the electric current, a power line 48 and the power line 44, for the operation of the restriction device 4. In accordance with one alternative, the capacitor 40 in the embodiment of FIG. 702-6 may be omitted. In accordance with another alternative, the accumulator 38 in this embodiment may be omitted.

FIG. 702-7 shows an embodiment identical to that of FIG. 702-1, except that a battery 50 for supplying energy for the operation of the restriction device 4 and an electric switch 52 for switching the operation of the restriction device 4 also are implanted in the patient. The switch 52 is operated by the energy supplied by the energy transforming means 6 to switch from an off mode, in which the battery 50 is not in use, to an on mode, in which the battery 50 supplies energy for the operation of the restriction device 4.

FIG. 702-8 shows an embodiment identical to that of FIG. 702-7, except that a control unit 36 controllable by the wireless remote control of the external energy transmission means 10 also is implanted in the patient. In this case, the switch 52 is operated by the energy supplied by the energy transforming means 6 to switch from an off mode, in which the wireless remote control is prevented from controlling the control unit 36 and the battery 50 is not in use, to a standby mode, in which the remote control is permitted to control the control unit 36 to release electric energy from the battery 50 for the operation of the restriction device 4. FIG. 702-9 shows an embodiment identical to that of FIG. 702-8, except that the accumulator 38 is substituted for the battery 50 and the implanted components are interconnected differently. In this case, the accumulator 38 stores energy from the energy transforming means 6. In response to a control signal from the wireless remote control of the external energy transmission means 10, the implanted control unit 36 controls the switch 52 to switch from an off mode, in which the accumulator 38 is not in use, to an on mode, in which the accumulator 38 supplies energy for the operation of the restriction device 4.

FIG. 702-10 shows an embodiment identical to that of FIG. 702-9, except that the battery 50 also is implanted in the patient and the implanted components are interconnected differently. In response to a control signal from the wireless remote control of the external energy transmission means 10, the implanted control unit 36 controls the accumulator 38 to deliver energy for operating the switch 52 to switch from an off mode, in which the battery 50 is not in use, to an on mode, in which the battery 50 supplies electric energy for the operation of the restriction device 4. Alternatively, the switch 52 may be operated by energy supplied by the accumulator 38 to switch from an off mode, in which the wireless remote control is prevented from controlling the battery 50 to supply electric energy and the battery 50 is not in use, to a standby mode, in which the wireless remote control is permitted to control the battery 50 to supply electric energy for the operation of the restriction device 4. FIG. 702-11 shows an embodiment identical to that of FIG. 702-7, except that a motor 15, a mechanical reversing device in the form of a gearbox 54 and a control unit 36 for controlling the gearbox 54 also are implanted in the patient. The implanted control unit 36 controls the gearbox 54 to reverse the function performed by the restriction device 4 (mechanically operated). FIG. 12 shows an embodiment identical to that of FIG. 702-10 except that the implanted components are interconnected differently. Thus, in this case the battery 50 powers the control unit 36 when the accumulator 38, suitably a capacitor, activates the switch 52 to switch to an on mode. When the switch 52 is in its on mode the control unit 36 is permitted to control the battery 50 to supply, or not supply, energy for the operation of the restriction device 4.

FIG. 702-13 schematically shows conceivable combinations of implanted components of the apparatus for achieving various communication options. Basically, there are the implanted restriction device 4, control unit 36 and motor/pump unit 18, and the external energy transmission means 10 including the external wireless remote control. As already described above the wireless remote control transmits a control signal which is received by the implanted control unit 36, which in turn controls the various implanted components of the apparatus. A sensor 56 may be implanted in the patient for sensing a physical parameter of the patient, such as the pressure in the passageway. The implanted control unit 36, or alternatively the external wireless remote control of the energy transmission means 10, may control the restriction device 4 in response to signals from the sensor 56. A transceiver may be combined with the sensor 56 for sending information on the sensed physical parameter to the external wireless remote control. The wireless remote control may comprise a signal transmitter or transceiver and the implanted control unit 36 may comprise a signal receiver or transceiver. Alternatively, the wireless remote control may comprise a signal receiver or transceiver and the implanted control unit 36 may comprise a signal transmitter or transceiver. The above transceivers, transmitters and receivers may be used for sending information or data related to the restriction device 4 from inside the patient's body to the outside thereof. Where the motor/pump unit 18 and battery 50 for powering the motor/pump unit 18 are implanted, the battery 50 may be equipped with a transceiver for sending information on the condition of the battery 50. Those skilled in the art will realize that the above various embodiments according to FIGS. 702-1-702-13 could be combined in many different ways. For example, the energy operated switch 702-14 could be incorporated in any of the embodiments of FIGS. 702-3, 702-6-702-12, the hydraulic shifting device 34 could be incorporated in the embodiment of FIG. 702-4, and the gearbox 54 could be incorporated in the embodiment of FIG. 702-3.

FIG. 702-14 shows the energy transforming means in the form of an electrical junction element 58 for use in any of the above embodiments according to FIGS. 702-1-702-13. The element 58 is a flat p-n junction element comprising a p-type semiconductor layer 60 and an n-type semiconductor layer 62 sandwiched together. A light bulb 64 is electrically connected to opposite sides of the element 58 to illustrate how the generated current is obtained. The output of current from such a p-n junction element 58 is correlated to the temperature. See the formula below.

$$I = 10 ( exp ( qV / kT ) - 1 )$$

I is the external current flow,
I0 is the reverse saturation current, q is the fundamental electronic charge of 1.602×10-19 columns,
V is the applied voltage, k is the Boltzmann constant, and T is the absolute temperature.

Under large negative applied voltage (reverse bias), the exponential term becomes negligible compared to 1.0, and I is approximately −10. 10 is strongly dependent on the temperature of the junction and hence on the intrinsic-carrier concentration. 10 is larger for materials with smaller bandgaps than for those with larger bandgaps. The rectifier action of the diode, that is, its restriction of current flow to only one direction, is in this particular embodiment the key to the operation of the p-n junction element 58. The alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers such as but not limited to cadmium telluride, copper-indium-diselenide and silicon. It is also possible to use a multilayer structure with several layers of p and n-type materials to improve efficiency. The electric energy generated by the p-n junction element 58 could be of the same type as generated by solar cells, in which the negative and positive fields create a direct current. Alternatively, the negative and positive semiconductor layers may change polarity following the transmitted waves, thereby generating the alternating current.

The p-n junction element 58 is designed to make it suited for implantation. Thus, all the external surfaces of the element 58 in contact with the human body are made of a biocompatible material. The p-n junction semiconductors are designed to operate optimally at a body temperature of 37° C. because the current output, which should be more than 1 μA, is significantly depending on temperature as shown above. Since both the skin and subcutis absorb energy, the relation between the sensitivity or working area of the element 58 and the intensity or strength of the wireless energy transmission is considered. The p-n junction element 58 preferably is designed flat and small. Alternatively, if the element 58 is made in larger sizes it should be flexible, in order to adapt to the patient's body movements. The volume of the element 58 should be kept less than 2000 cm3.

FIG. 702-15 generally illustrates how any of the above-described embodiments of the impotence treatment apparatus may be implanted in a male patient. Thus, a restriction device 4 implanted in a patient engages penile tissue or the prolongation thereof to form an artificial sphincter around the passageway through which the blood flow leaves the penis. An implanted operation device 68, which may also be referred to as an adjustment device, such as an electric motor or a motor/pump assembly, operates the restriction device 4 through a transmission member 70, such as a mechanical transmission cord or a fluid tube. The energy transforming means in the form of the element 6 having a positive region and a negative region, as described above in more detail, is placed underneath the skin of the patient. The external energy transmission means 10 is adapted to transmit wireless energy to the implanted element 6. A wireless remote control includes an external control unit incorporated in the energy transmission means 10, and an implanted control unit 71 connected to the element 6 and the operation device 68. Wireless energy carried by a signal transmitted by the wireless remote control of the external energy transmission means 10 at least partly penetrates the patient's skin and hits the element 6. The energy thus hitting the element 6 is transformed into energy of a different form that is suited for powering the operation device 68. For example, where the operation device 68 is an electric motor the element 6 comprises an electric p-n junction element that transforms the wireless energy into an electric current for powering the electric motor. Where the operation device 68 comprises a pump, the element 6 may transform the wireless energy into kinetic energy for powering the pump. The transformed energy may be utilized for directly operating the restriction device 4 or, where the restriction device 4 is electrically operated, for storage in a capacitor and/or an accumulator for later or parallel use. Preferably (but not necessarily) the element 6 is controlled by a microprocessor. The wireless remote control of the external energy transmission means 10 is used to control the utilization of the transmitted energy and any function or command to/from the implanted restriction device 4.

FIG. 702-16 shows the basic parts of a wireless remote control of the apparatus including an electric motor 128 for operating a restriction member, for example of the type illustrated in FIG. 702-15. In this case, the remote control is based on the transmission of electromagnetic wave signals, often of high frequencies in the order of 100 kHz-1 gHz, through the skin 130 of the patient. In FIG. 702-15, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134.

An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either contract or enlarge the restriction device. The microprocessor starts a command by applying a high frequency signal on the antenna 132. After a short time, when the signal has energized the implanted parts of the control system, commands are sent to contract or enlarge the restriction device in predefined steps. The commands are sent as digital packets in the form illustrated below.

| | | | |
|---|---|---|---|
| Start pattern, | Command, | Count, | Checksum, |
| 8 bits | 8 bits | 8 bits | 8 bits |

The commands are sent continuously during a rather long time period (e.g. about 30 seconds or more). When a new contract or enlarge step is desired the Count byte is increased by one to allow the implanted control unit 138 to decode and understand that another step is demanded by the external control unit 136. If any part of the digital packet is erroneous, its content is simply ignored. Through a line 140, an implanted energizer unit 126 draws energy from the high frequency electromagnetic wave signals received by the receiving antenna 134. The energizer unit 126 stores the energy in an energy storage device, such as a large capacitor, powers the control unit 138 and powers the electric motor 128 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to either contract or enlarge the restriction device depending on the received command code. Alternatively, the energy stored in the energy storage device of the energizer unit may only be used for powering a switch, and the energy for powering the motor 128 may be obtained from another implanted energy source of relatively high capacity, for example a battery. In this case the switch is adapted to connect said battery to the control unit 138 in an on mode when said switch is powered by the energy storage device and to keep the battery disconnected from the control unit in a standby mode when the switch is not powered. With reference to FIG. 702-17, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178.

The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### AA: Male Impotence Treatment

FIGS. 703-1A-D are—a block diagrams of four different embodiments of the male sexual impotence treatment apparatus according to the invention. FIG. 703-1A shows an elongated restriction member in the form of a band 2 forming a loop which defines a restriction opening. The band 2 provides a restricted blood through-flow area in the corpora cavernosa when applied around the latter. FIG. 1A further shows a separate reservoir 4, a one way pump 6 and an alternate valve 8. FIG. 703-1B shows the band 2 and a fluid supply reservoir 10. FIG. 703-1C shows the band 2, a two-way pump 12 and the reservoir 4. FIG. 703-1D shows a servo system with a first closed system controlling a second system. The servo system comprises the fluid supply reservoir 10 and a servo reservoir 14. The servo reservoir 14 controls a larger adjustable reservoir 16 which in connection with the band 2 applied around the corpora cavernosa varies the volume of a cavity in the band, which in turn varies the restricted blood through-flow area in the corpora cavernosa. Such a band 2 forming the restriction opening 3 is illustrated schematically in FIG. 703-8. The band 2 comprises an adjustment device having an expandable/contractable cavity 5 which is expanded or contracted by supplying hydraulic fluid (e.g. from reservoir 4, 6, 10, or 16), and the band 2 may be sutured in place, illustrated schematically at 7 in FIG. 703-8.

FIGS. 703-2A-D are cross-sectional views of a pump mechanism adapted to pump fluid in both directions only by mechanically pushing a separate sealing wall portion 18 in one direction. FIG. 703-2A shows a piston 20 pushed forwards against a spring 22 towards the wall portion 18 and located in a pump housing 24 conducting fluid from a right upper fluid passage 26 of the housing 24 to a left fluid passage 28 of the housing 24. A main valve 30 is open and a non-return valve 32 is closed. FIG. 703-2B illustrates the first pump movement in which the piston 20 has moved forwards and reaches the wall portion 18. FIG. 703-2C illustrates how the piston 20 moves backwards by the action of the spring 22. The main valve 30 is now closed and the non-return valve 32 is open for fluid from the right upper passage 26. FIG. 703-1D illustrates how the piston 20 is moved further downwards from its position according to FIG. 703-2B while pushing the wall portion 18 downwardly against a second spring 34 that is stronger than spring 22, whereby fluid escapes from a right lower fluid passage 36. When moving the piston 20 backwardly from the position according to FIG. 703-2D, fluid enters the left fluid passage 28 and a valve 38 in the lower right fluid passage 36 closes.

FIG. 703-3 is a cross-sectional view of a reservoir 40 defining a chamber 42, the volume of which is variable and is controlled by a remote controlled electric motor 44, in accordance with FIG. 703-1B or 703-1D. The reservoir 40 and the motor 44 are placed in a housing 46. The chamber 42 is varied by moving a large wall 48. The wall 48 is secured to a nut 50, which is threaded on a spindle 52. The spindle 52 is rotated by the motor 44 via an angular gearing, which comprises two conical gear wheels 54 and 56 in mesh with each other. The motor 44 is powered by a battery 58 placed in the housing 46. An signal receiver 60 for controlling the motor 44 is also placed in the housing 46. Alternatively, the battery 58 and the signal receiver 60 may be mounted in a separate place. The motor 44 may also be powered by energy transferred from transmitted signals.

FIG. 703-4 is a cross-sectional view of a reservoir 62 defining a chamber 64, the volume of which is variable and is controlled by manual manipulation. A gable wall portion 66 of an open ended inner cylindrical housing 68 is adapted to be pushed downwards to fit in a desired locking groove 70 of a plurality of locking grooves 70 on the mantle wall of the cylindrical housing 68, to reduce the volume of the chamber 64. The inner cylindrical housing 68 is suspended by springs 72 and is telescopically applied on an outer cylindrical housing 74. When pushing the inner cylindrical housing 68 it moves downwards relative to the outer cylindrical housing 74 causing the gable wall portion 66 to release from the locking groove 70 and move upwards relative to the inner cylindrical housing 68. When the inner housing 68 is moved upwardly by the action of the springs 72 the volume of the chamber 64 is increased.

FIGS. 703-5A and 703-5B show a servo means comprising a main ring-shaped fluid reservoir 76 defining a chamber 78, the volume of which is variable. Centrally positioned in the main ring-shaped reservoir 76 there is a servo fluid reservoir 80 defining a chamber 82, the volume of which is variable. The chamber 82 of the servo reservoir 80 is substantially smaller than the chamber 78 of the main reservoir 76. The two reservoirs 76 and 80 are situated between two opposite separate walls 84 and 86, and are secured thereto. When changing the amount of fluid in the servo reservoir 80, the two opposite walls 84, 86 are moved towards or away from each other, whereby the volume of the chamber 78 of the main reservoir 76 is changed.

FIG. 703-6 shows the basic parts of a remote control system of the apparatus including the electric motor 44 of the embodiment shown in FIG. 703-3. In this case, the remote control system is based on the transmission of electromagnetic wave signals, often of high frequencies in the order of 100 kHz-1 gHz, through the skin 130 of the patient. In FIG. 703-6, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134. An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either increase or decrease the size of the restriction opening defined by the loop of the restriction member 2. The microprocessor starts a command by applying a high frequency signal on the antenna 132. After a short time, when the signal has energized the implanted parts of the control system, commands are sent to increase or decrease the size of the restriction opening of the restriction member 2 in predefined steps. The commands are sent as digital packets in the form illustrated below.
Start Command Count, Checks pattern,
8 bits, 8 bits 8 bits urn, 8 bits

The commands are sent continuously during a rather long time period (e.g. about 30 seconds or more). When a new increase or decrease step is desired the Count byte is increased by one to allow the implanted control unit 138 to decode and understand that another step is demanded by the external control unit 136. If any part of the digital packet is erroneous, its content is simply ignored. Through a line 140, an implanted energizer unit 126 draws energy from the high frequency electromagnetic wave signal received by the receiving antenna 134. The energizer unit 126 stores the energy in a power supply, such as a large capacitor, powers the control unit 138 and powers the electric motor 44 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 44 to either increase or decrease the size of the restriction opening of the restriction member 2 depending on the received command code. Alternatively, the energy stored in the power supply of the energizer unit may only be used for powering a switch, and the energy for powering the motor 44 may be obtained from another implanted power source of relatively high capacity, for example a battery. In this case the switch is adapted to connect the battery to the control unit 138 in an on mode when the switch is powered by the power supply and to keep the battery disconnected from the control unit in a standby mode when said switch is unpowered. With reference to FIG. 703-7, the remote control system schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the restriction device. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 44 via an H-bridge 178 comprising transistors 180, 182, 184 and 186. The motor 44 can be driven in two opposite directions by the H-bridge 178. The microprocessor 176 also monitors the amount of stored energy in the storage capacitor 158. Before sending signals to activate the motor 44, the microprocessor 176 checks whether the energy stored in the storage capacitor 158 is enough. If the stored energy is not enough to perform the requested operation, the microprocessor 176 waits for the received signals to charge the storage capacitor 158 before activating the motor 44.

FIGS. 703-9A and 703-9B show an embodiment of the apparatus comprising a restriction device 202 having an elongated flexible restriction member 204, such as a belt, a cord or the like. The flexible member 204 extends in a loop around the corpora cavernosa. (Alternatively, the flexible member 204 may comprise two separate parts on opposite sides of the corpora cavernosa.) One portion 204A of member 204 is attached to a frame 208 and another portion 204B of member 204 opposite portion 204A in the loop of the flexible member 204 is connected to an adjustment device 210, which is fixed to the frame 208. The adjustment device 210 pulls the flexible member 204 away from portion 204A to squeeze the corpora cavernosa between two opposite lengths of the flexible member 204 to thereby restrict the blood flow leaving the penis, see FIG. 703-9A, and releases the corpora cavernosa from the flexible member 204 to thereby increase the blood flow leaving the penis, see FIG. 703-9B.

FIGS. 703-10A and 703-10B show an embodiment of the apparatus comprising a restriction device 212 having two plate or bar elements 214 on opposite sides of the corpora cavernosa 206. An adjustment device 216 moves the elements 212 in parallel towards each other to squeeze the corpora cavernosa 206 between the elements 212 to thereby restrict the blood flow leaving the penis, see FIG. 703-10A, and moves the elements 212 away from each other to increase the blood flow leaving the penis, see FIG. 703-10B.

FIG. 703-11 shows an embodiment of the apparatus comprising a restriction device 218 having two articulated clamping elements 220 positioned on opposite sides of the corpora cavernosa 206. An adjustment device 222 moves the clamping elements 220 toward each other to clamp the corpora cavernosa 206 between the clamping elements 220 to thereby restrict the blood flow leaving the penis, and moves the clamping elements 220 away from each other to release the corpora cavernosa 206 from the clamping elements 220 to thereby increase the blood flow leaving the penis.

FIGS. 703-12A, 703-12B and 703-12C show an embodiment of the apparatus comprising a restriction device 224 having three bending members in the form of cylindrical rollers 226, 228 and 230 displaced relative one another in a row along the corpora cavernosa 206 and positioned alternately on opposite sides of the corpora cavernosa 206. (Alternatively, each roller 226, 228 and 230 may take the shape of an hourglass.) An adjustment device 232 moves the two outer rollers 226, 230 laterally against the corpora cavernosa 206 in one direction and the intermediate roller 228 against the corpora cavernosa 206 in the opposite direction to bend the corpora cavernosa to thereby restrict the blood flow leaving the penis, see FIG. 703-12B. To release the corpora cavernosa from the rollers 226-230, the adjustment device 232 moves the rollers 226-230 away from the corpora cavernosa 206, see FIG. 703-12C.

FIGS. 703-13A through 703-17B schematically illustrates modifications of the above embodiment according to FIGS. 703-12A-703-12C. Thus, FIGS. 1703-3A and 703-13B show an embodiment similar to that of FIGS. 703-12A-703-12C except that the bending members are oval and not rotatable. FIGS. 703-14A and 703-14B show an embodiment similar to that of FIGS. 703-13A and 703-13B except that the oval bending members are rotatable to squeeze the corpora cavernosa, see FIG. 703-14B, and to release the corpora cavernosa, see FIG. 703-14A. FIGS. 703-15A and 703-15B show an embodiment similar to that of FIGS. 703-12A-703-12C except that the intermediate roller has a changeable diameter to squeeze the corpora cavernosa, see FIG. 703-15B, and to release the corpora cavernosa, see FIG. 703-15A. FIGS. 703-16A and 703-16B show an embodiment similar to that of FIGS. 703-10A-703-10C except that the elements are replaced by two cylindrical rollers positioned on opposite sides of the corpora cavernosa. Finally, FIGS. 703-17A and 703-17B show an embodiment substantially similar to that of FIGS. 703-16A and 703-16B except that the restriction device is turned 90 to form an S-shaped curvature of the corpora cavernosa. FIG. 703-18 shows an example of a hydraulic restriction device 234 for use in accordance with the invention. The restriction device 234 comprises an elongated restriction member 236 having an inflatable cavity 238. A tube 240 connects the cavity 238 to a hydraulic fluid reservoir, not shown. The restriction member 236 may be wrapped around the corpora cavernosa. Alternatively, two restriction members 236 may be wrapped around the respective crura.

FIG. 703-19 schematically illustrates how any of the above-described embodiments of the male sexual impotence treatment apparatus can be implanted in a patient. Thus, an implanted adjustable hydraulic restriction device 234 extends almost completely around the corpora cavernosa 242 and the urethra 244 to be capable of contracting the corpora cavernosa 242 as a single unit. An adjustment device in the form of an inflatable cavity in the restriction device 234 is adapted to adjust the restriction device 234 so that the blood flow leaving the penis is restricted. An implanted assembly 246 includes a hydraulic fluid reservoir and a hydraulic operation device (which may include a pump) for distributing hydraulic fluid between the reservoir and the inflatable/contractible cavity of the restriction device 234 via a fluid conduit 240.

A wireless remote control of the apparatus comprises an external signal transmitter 248, which may comprise a hand-held unit, and an implanted signal receiver, which is incorporated in the implanted assembly 246. The assembly 246 further includes a control unit for controlling the restriction device 234 in response to a control signal from the external transmitter. The signal receiver of the assembly 246 further includes an energizer unit which transforms energy from the control signal transmitted by the external transmitter into electric energy for powering the operation device of the assembly 246 and for energy consuming implanted components of the apparatus.

A pressure sensor 250 is implanted for sensing the pressure on the restriction device 234. The control unit of the assembly 246 controls the operation device of the assembly 246 to operate the adjustment device 234 to adjust the restriction device 234 so that the corpora cavernosa is released in response to the pressure sensor 250 sensing an abnormal high pressure. There is a number of conceivable alternative embodiments that give the same result as the above-described embodiments. For example, the microprocessor of the external and implanted, respectively, control units may be replaced by discrete components. The power amplifier of the external control unit may be omitted if the signals generated by the signal generator are strong enough. Therefore, the invention is to be accorded the broadest interpretation of the appended claims to encompass all equivalent structures and assemblies.

### AB: Impotence Treatment Apparatus

FIGS. 704-1-704-3 show a preferred embodiment of the male sexual impotence treatment apparatus comprising a restriction device having an elongated restriction member in the form of a circular resilient core 2 with two overlapping end portions 4, 6. The core 2 defines a substantially circular restriction opening and is enclosed in an elastic soft hose 8 except at a releasable and lockable joint 10 of the core 2, which when released enables application of the core 2 with its hose 8 around a tissue of a patient, such as the corpus cavernosum or one or more exit veins from the patient's penis. The materials of all of these elements are bio-compatible so that the patient's body will not reject them. A mechanical adjustment device 12 for mechanically adjusting the longitudinal extension of the core 2 to change the size of the restriction opening comprises a drive wheel 14 in frictional engagement with the overlapping end portions 4, 6 of the core 2. The drive wheel 14 is journalled on a holder 16 placed in the hose 8 and provided with two counter pressure rollers 18, 20 pressing the respective end portions 4, 6 of the core 2 against the drive wheel 14 to increase the frictional engagement therebetween. An electric motor 22 is connected to the drive wheel 14 via a long flexible drive shaft 24 and is moulded together with a remote controlled power supply unit 26 in a body 28 of silicone rubber. The length of the flexible drive shaft 34 is selected so that the body 28 can be placed in a desired position in the patient's body, suitably in the abdomen.

When the patient wishes to achieve erection, he controls the power supply unit 26 to power the electric motor 22 to turn the drive wheel 14 in one direction to reduce the diameter of the core 2, so that the penile tissue is squeezed and the blood flow leaving the penis is restricted. When the patient wishes to regain flaccid condition of his penis, he controls the power supply unit 26 to power the electric motor 22 to turn the drive wheel 14 in the opposite direction to increase the diameter of the core 2, so that the tissue is released. Alternatively, a rack gear may be formed on one of the end portions 4, 6 of the core 2 and the drive wheel 14 may be replaced by a drive gear wheel connected to the other end portion of the core 2 and in mesh with the rack gear.

FIG. 704-4 shows an embodiment which is identical to the embodiment of FIGS. 704-1-704-3, except that the motor 22 is encapsulated in a lateral protrusion 30 of the hose 8 so that it is fixed to the core 2 and has a short drive shaft 32 onto which the drive wheel 14 is mounted, and that the motor 22 is positioned relative to the circular core 2 such that the drive shaft 32 extends radially thereto.

FIG. 704-5 shows an embodiment which likewise is identical to the embodiment of FIGS. 704-1-704-3, except that the motor 22 is encapsulated in the hose 8 so that it is fixed to the core 2 and has a short drive shaft 32, and that the motor 22 is positioned relative to the core 2 such that the drive shaft 32 extends substantially tangentially to the circular core 2. There is an angular gearing 34 connecting the drive shaft 32 to the drive wheel 14.

FIG. 704-6 shows a suitable arrangement for the motor 22 in the embodiment of FIG. 704-5, comprising a first clamping member 36 secured to one end portion of the core 2 and a second clamping member 38 secured to the other end portion 6 of the core 2. The motor 22 is secured to the first clamping member 36 and is operatively connected to a worm 40 via a gear transmission 42. The worm 40 is journalled at its opposite ends on holders 44 and 46, which are rigidly secured to the clamping member 36 and the motor 22, respectively. The second clamping member 38 has a pinion in mesh with the worm 40. When the motor 22 is powered the worm 40 rotates and will thereby pull the end portion 6 of the core 2 in one or the opposite longitudinal direction, so that the diameter of the substantially circular core 2 is either increased or decreased.

FIG. 704-7 shows an embodiment in which the elongated restriction member comprises a core 48 and a helical spring 50. A spring contracting means in the form of a flexible pulling member 52, i.e. a string, wire or cable, is connected to the core 48 at one end thereof and extends through the helical spring 50. A hydraulic motor in the form of a cylinder/piston unit 54 is adapted to pull the flexible pulling member 52 to contract the helical spring 50 against an arresting member 56, which is fixed relative to the core 48. A tube 58 hinged to the arresting member 56 extends between the cylinder/piston unit 54 and the arresting member 56, the flexible pulling member 52 running through the tube 58 and being connected to the piston of the cylinder/piston unit 54. FIG. 704-8 shows a similar embodiment in which a hydraulic transmission conduit 59 is provided between two piston-cylinder assemblies 54, for use as the hydraulic motor/device in FIG. 704-7.

FIG. 704-9 shows an embodiment in which the restriction member comprises two elongated helical springs 60 and 62 having free ends, and a body 64 to which the springs 60, 62 are nonrotatably secured at their opposite ends. The body 64 comprises two separate parts secured to opposite end portions of the enclosing elastic hose 8 and is designed with a releasable and lockable joint between the separate parts. An adjustment device in the form of a drive shaft 66 has two opposite end portions connected to the helical springs 60, 62, respectively, at their free ends. The coils of the springs 60, 62 form left and right hand helices, respectively. A motor 68 is adapted to rotate the drive shaft 66 in one direction to enlarge the coils of the helical springs 60, 62 to longitudinally contract the springs 60, 62 and to rotate the drive shaft 66 in the opposite direction to reduce the size of the coils of the springs 60, 62 to longitudinally extend the springs 60, 62. Thus, the elongated helical springs 60, 62 defines a restriction opening, the size of which is increased when the springs 60, 62 are extended and decreased when the springs 60, 62 are contracted.

FIG. 704-10 shows an embodiment according to the invention which is identical to the embodiment of FIG. 704-9, except that the adjustment device comprises a gearing having an input shaft 72 and two opposite aligned output shafts 74 and 76 connected to the helical springs 60 and 62, respectively, at their free ends. The input shaft 72 is connected to the output shafts 74, 76 such that they rotate at opposite directions upon rotation of the input shaft 72. The coils of the springs 60, 62 form the same helices.

FIGS. 704-11-704-14 show an embodiment of the device in which a hydraulic motor comprises two interconnected cylinders 78 and 80 and two pistons 82 and 84 in the respective cylinders 78, 80. The cylinders 78, 80 have a common fluid supply inlet member 86, which together with the cylinders 78, 80 takes the shape of a Y-pipe. The restriction member comprises an elongated resilient arcuate core 88. The adjustment device comprises two bars 90 and 92 secured to opposite ends of the core 88 and connected to the pistons 82 and 84, respectively. The core 88 defines a restriction opening and is provided with a releasable and lockable joint 94 (FIG. 704-13) to permit application of the core 88 around the tissue. The core 88 and the cylinders 90, 92 are enclosed by a soft elastic hose 96 except at the joint 94 and the inlet member 86. The hose 96 has an outer tubular wall 98 and a central coaxial inner tubular wall 100, which is fixed to the outer wall 98 by spoke members 102 (FIG. 704-14). The core 88 is loosely fit in the inner tubular wall 100. By supplying fluid to or withdrawing fluid from the inlet 86 the pistons 82 and 84 will move towards or from each other, so that the restriction opening defined by the core 88 is changed by the longitudinal displacement of the bars 90, 92.

FIGS. 704-15-704-17 show an embodiment which is identical to the embodiment of FIGS. 704-11-704-14, except that the adjustment device comprises an elongated voltage responsive element 104 secured to the opposite ends of the core 88, so that the core 88 and the element 104 form the restriction member. The element 104 is capable of bending inwardly into a bow in response to a voltage applied across the element 104. The radius of curvature of said bow is adjustable by changing the level of the voltage applied to element 104.

FIG. 704-18 shows an embodiment comprising a loop forming means in the form of a substantially rigid outer circular element 106 with a releasable and lockable joint 108. In this embodiment the restriction member comprises an elastic inner circular element 110 formed by the innermost wall portion of an elastic hose 112 extending along the outer element 106. The inner circular element 110 is disposed concentrically within the outer circular element 106. The adjustment device comprises a plurality of threads 114 secured to the elastic inner element 110 along the circumference thereof and running from the inner element 110 via guide, members 116 attached to the outer element 106. By pulling all the threads 114 the inner elastic element 110 is pulled under expansion radially outwardly towards the outer element 106.

FIG. 704-19 shows an embodiment which is identical to the embodiment of FIG. 704-9, except that it comprises a loop forming means in the form of a substantially rigid outer circular element 118 supporting the helical springs 60, 62, and a soft elastic inner wall 120 extending along the springs 60, 62. When the motor 68 rotates the helical springs 60, 62 in a direction that enlarges the coils of the springs 60, 62, the coils are forced by the rigid outer element 118 to expand radially inwardly thereby reducing the size of the restriction opening formed by the circumferential confinement surface of the restriction member (springs 60, 62 and body 64).

FIG. 704-20 shows an embodiment in which a restriction member comprises a plurality of arcuate lamellae 122 arranged like the conventional adjustable aperture mechanism of a camera. The adjustment device, not shown, is conventional and is operated by a motor 124 to adjust the lamellae 122 to change the size of an restriction opening defined by the lamellae 122.

FIGS. 704-21-704-23 show an embodiment in which a restriction member comprises two semi-circular elements 126 and 128 which are hinged together such that the semi-circular elements 126, 128 are swingable relative to each other between a fully open state in which they substantially form a circle, illustrated in FIG. 704-22 and an angular state, in which the size of the restriction opening defined by the semi-circular elements 126, 128 is reduced, illustrated in FIG. 704-23. The adjustment device, not shown, is conventional and is operated by a motor 130 to swing the semi-circular elements 126, 128 relative to each other.

FIGS. 704-24-704-27 show an embodiment in which a restriction member comprises an elastic belt 130 forming a circle and having a substantially oval cross-section. The restriction member 130 is provided with a releasable and lockable joint 132. An elastic double walled hose 134 encloses the belt 130 except at the joint 132. The adjustment device, not shown, is conventional and is operated by a motor 136 to turn the belt 130 around the longitudinal extension thereof between a fully open state, in which the inner broader side of the belt 130 forms a substantially cylindrical surface, illustrated in FIG. 704-26, and a reduced open state, in which the inner broader side of the belt 130 forms a substantially conical surface, illustrated in FIG. 704-27.

FIG. 704-28 schematically illustrates a cushion arrangement for protecting the penile tissue, comprising a plurality of cushions 138 disposed in series along a substantially circular holding member 140. This cushion arrangement may be utilized in any of the above described embodiments of the invention.

FIGS. 704-29A-D provide a block diagram of four different hydraulic transmission configurations. FIG. 704-29A shows an adjustment device 202, a separate reservoir 204, a one way pump 206 and an alternate valve 208. FIG. 704-29B shows the adjustment device 202 and an adjustable reservoir 210. FIG. 704-29C shows the adjustment device 202, a two-way pump 212 and the reservoir 204. FIG. 704-29D shows a reverse servo system with a first closed system controlling a second system. The servo system comprises an adjustable servo reservoir 210 and a passive adjustable fluid supply reservoir 214. Any of the reservoirs can be the active reservoir, either the servo reservoir 210 or the fluid supply reservoir 214. The reservoir 214 controls a larger adjustable reservoir 216, which is used for the operation of the adjustment device 202 for changing the restriction opening of the restriction member.

FIGS. 704-30A-D are cross-sectional views of a pump mechanism adapted to pump fluid in both directions only by mechanically pushing a separate sealing wall portion 218 in one direction. FIG. 30A shows a piston 220 pushed forwards against a spring 222 towards the wall portion 218 and located in a pump housing 224 conducting fluid from a right upper fluid passage 226 of the housing 224 to a left fluid passage 228 of the housing 224. A main valve 230 is open and a nonreturn valve 232 is closed.

FIG. 704-30B illustrates the first pump movement in which the piston 220 has moved forwards and reaches the wall portion 218. FIG. 704-30C illustrates how the piston 220 moves backwards by the action of the spring 222. The main valve 230 is now closed and the nonreturn valve 232 is open for fluid from the right upper passage 226. FIG. 704-30D illustrates how the piston 220 is moved further downwards from its position according to FIG. 704-30B while pushing the wall portion 218 downwards against a second spring 234 that is stronger than spring 222, so that fluid escapes from a right lower fluid passage 236. When moving the piston 220 backwards from the position of FIG. 704-30D, fluid enters the left fluid passage 228 and a valve 238 in the lower right fluid passage 236 closes.

FIG. 704-31 is a cross-sectional view of a reservoir 240 defining a chamber 242, the size of which is variable and is controlled by a remote controlled motor 244, in accordance with FIG. 704-29B or 29D. The reservoir 240 and the motor 244 are placed in a housing 246. The chamber 242 is varied by moving a large wall 248. The wall 248 is secured to a nut 250, which is threaded on a rotatable spindle 252. The spindle 252 is rotated by the motor 244 via an angular gearing, which comprises two conical gear wheels 254 and 256 in mesh with each other. The motor 244 is powered by a battery 258 placed in the housing 246. A signal receiver 260 for controlling the motor 244 is also placed in the housing 246. Alternatively, the battery 258 and the signal receiver 260 may be mounted in a separate place. The signal receiver may comprise any known or conventional device which is capable of receiving a control signal and then operating the motor 244. FIG. 704-32 is a cross-sectional view of a reservoir 262 defining a chamber 264, the size of which is variable and is controlled by manual manipulation. A gable wall portion 266 of an open ended inner cylindrical housing 68 is adapted to be pushed downwards to fit in a desired locking groove 270 of a plurality of locking grooves 270 on the mantle wall of the cylindrical housing 268, to reduce the size of the chamber 64. The inner cylindrical housing 268 is suspended by springs 272 and is telescopically applied on an outer cylindrical housing 274. When pushing the inner cylindrical housing 268 it moves downwards relative to the outer cylindrical housing 274 causing the gable wall portion 266 to release from the locking groove 270 and move upwards relative to the inner cylindrical housing 268. When the inner housing 268 is moved upwardly by the action of the springs 272 the size of the chamber 264 is increased.

FIGS. 704-33A and 704-33B show a reverse servo means comprising a main ring-shaped fluid reservoir 276 defining a chamber 278, the size of which is variable. Centrally positioned in the main ring-shaped reservoir 276 there is a servo fluid reservoir 280 defining a chamber 282, the size of which is variable. The chamber 282 of the servo reservoir 280 is significantly smaller than the chamber 278 of the main reservoir 276. The two reservoirs 276 and 280 are situated between two opposite separate walls 284 and 286, and are secured thereto. When changing the amount of fluid in the servo reservoir 280, the two opposite walls 284, 286 are moved towards or away from each other, whereby the size of the chamber 278 of the main reservoir 276 is changed.

FIG. 704-34 shows the basic parts of a remote control system of the apparatus including a motor, for instance the electric motor 22. In this case, the remote control system is based on the transmission of an electromagnetic wave signal, often of a high frequency in the order of 100 kHz-1 gHz, through the skin 330 of the patient. In FIG. 704-34, all parts placed to the left of the skin 330 are located outside the patient's body and all parts placed to the right of the skin 330 are implanted in the patient's body.

An external signal transmitting antenna 332 is to be positioned close to a signal receiving antenna 334 implanted in the patient's body close to the skin 330. As an alternative, the receiving antenna 334 may be placed for example inside the abdomen of the patient. The receiving antenna 334 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 332 comprises a coil having about the same size as the coil of the receiving antenna 334 but wound with a thick wire that can handle the larger currents that is necessary. The coil of the transmitting antenna 332 is tuned to the same specific high frequency as the coil of the receiving antenna 334.

An external control unit 336 comprises a microprocessor, a high frequency electromagnetic signal generator and a power amplifier. The microprocessor of the control unit 336 is adapted to switch on/off the generator and to modulate signals generated by the generator to send digital information via the power amplifier and the antennas 332, 334 to an implanted control unit 338. To avoid that accidental random high frequency fields trigger control commands, digital signal codes are used. A keypad placed on the external control unit 336 is connected to the microprocessor thereof. The keypad is used to order the microprocessor to send a digital signal to either increase or decrease the size of the restriction opening defined by the loop of the restriction member (e.g. as described above). The microprocessor starts a command by applying a high frequency signal on the antenna 332. After a short time, when the signal has energized the implanted parts of the control system, commands are sent to increase or decrease the size of the restriction opening of the restriction member in predefined steps. The commands are sent as digital packets in the form illustrated below.
Pattern, Command, Count, Checksum, 8 bits 8 bits 8 bits 8 bits

The commands are sent continuously during a rather long time period {e.g. 30 seconds or more). When a new increase or decrease step is desired the Count byte is increased by one to allow the implanted control unit 338 to decode and understand that another step is demanded by the external control unit 336. If any part of the digital packet is erroneous, its content is simply ignored. Through a line 340, an implanted energizer unit 326 draws energy from the high frequency electromagnetic wave signal received by the receiving antenna 334. The energizer unit 326 stores the energy in a power supply, such as a large capacitor, powers the control unit 338 and powers the electric motor 22 via a line 342. The control unit 338 comprises a demodulator and a microprocessor. The demodulator demodulates digital signals sent from the external control unit 336. The microprocessor of the control unit 338 receives the digital packet, decodes it and, provided that the power supply of the energizer unit 326 has sufficient energy stored, sends a signal via a signal line 344 to the motor 22 to either increase or decrease the size of the restriction opening of the restriction member depending on the received command code. Alternatively, the energy stored in the power supply of the energizer unit may only be used for powering a switch, and the energy for powering the motor 22 may be obtained from another implanted power source of relatively high capacity, for example a battery. In this case the switch is adapted to connect the battery to the control unit 338 in an “on” mode when said switch is powered by said power supply and to keep said battery disconnected from the control unit in a “standby” mode when the switch is not powered. With reference to FIG. 35, the remote control system schematically described above will now be described in accordance with a more detailed embodiment. The external control unit 336 comprises a microprocessor 346, a signal generator 348 and a power amplifier 350 connected thereto. The microprocessor 346 is adapted to switch the signal generator 348 on/off and to modulate signals generated by the signal generator 348 with digital commands that are sent to implanted components of the device of the invention. The power amplifier 350 amplifies the signals and sends them to the external signal transmitting antenna 332. The antenna 332 is connected in parallel with a capacitor 352 to form a resonant circuit tuned to the frequency generated by the signal generator 348. The implanted signal receiving antenna coil 334 forms together with a capacitor 354 a resonant circuit that is tuned to the same frequency as the transmitting antenna 332. The signal receiving antenna coil 334 induces a current from the received high frequency electromagnetic waves and a rectifying diode 360 rectifies the induced current, which charges a storage capacitor 358. A coil 356 connected between the antenna coil 334 and the diode 360 prevents the capacitor 358 and the diode 360 from loading the circuit of the signal receiving antenna 334 at higher frequencies. Thus, the coil 356 makes it possible to charge the capacitor 358 and to transmit digital information using amplitude modulation. A capacitor 362 and a resistor 364 connected in parallel and a diode 366 forms a detector used to detect amplitude modulated digital information. A filter circuit is formed by a resistor 368 connected in series with a resistor 370 connected in series with a capacitor 372 connected in series with the resistor 368 via ground, and a capacitor 374, one terminal of which is connected between the resistors 368, 370 and the other terminal of which is connected between the diode 366 and the circuit formed by the capacitor 362 and resistor 364. The filter circuit is used to filter out undesired low and high frequencies. The detected and filtered signals are fed to an implanted microprocessor 376 that decodes the digital information and controls the motor 22 via an H-bridge 378 comprising transistors 380, 382, 384 and 386. The motor 22 can be driven in two opposite directions by the H-bridge 378. The microprocessor 376 also monitors the amount of stored energy in the storage capacitor 358. Before sending signals to activate the motor 22, the microprocessor 376 checks whether the energy stored in the storage capacitor 358 is enough. If the stored energy is not enough to perform the requested operation, the microprocessor 376 waits for the received signals to charge the storage capacitor 358 before activating the motor 22.

FIGS. 704-36A and 704-36B show an embodiment of the apparatus comprising a restriction device 402 having an elongated flexible restriction member 404, such as a belt, a cord or the like. The flexible member 404 extends in a loop around the tissue, suitably an exit vein from the penis. (Alternatively, the flexible member 404 may comprise two separate parts on opposite sides of the vein.) One portion 404A of member 404 is attached to a frame 408 and another portion 404B of member 404 opposite portion 404A in the loop of the flexible member 404 is connected to an adjustment device 410, which is fixed to the frame 408. The adjustment device 410 pulls the flexible member 404 in the direction from portion 404A to squeeze the vein between two opposite lengths of the flexible member 404 to thereby restrict the blood flow in the vein 406, see FIG. 704-36A, and releases the vein from the flexible member 404 to thereby increase the blood flow in the vein 406, see FIG. 704-36B.

FIGS. 704-37A and 704-37B show an embodiment of the apparatus comprising a restriction device 412 having two rigid plate or bar elements 414 on opposite sides of the vein 406. An adjustment device 416 moves the rigid elements 412 in parallel towards each other to squeeze the vein 406 between the rigid elements 412 to thereby restrict the blood flow in the vein 406, see FIG. 704-37A, and moves the rigid elements 412 away from each other to release the vein 406, see FIG. 704-37B. FIG. 704-38 shows an embodiment of the apparatus comprising a restriction device 418 having two rigid articulated clamping elements 420 positioned on opposite sides of the vein 406. An adjustment device 422 turns the clamping elements 420 toward each other to clamp the vein 406 between the clamping elements 420 to thereby restrict the blood flow in the vein 406, and turns the clamping elements 420 away from each other to release the vein 406 from the clamping elements 420 to thereby increase the blood flow in the vein 406.

FIGS. 704-39A, 704-39B and 704-39C show an embodiment of the apparatus comprising a restriction device 424 having three bending members in the form of cylindrical rollers 426, 428 and 430 displaced relative one another in a row along the vein 406 and positioned alternately on opposite sides of the vein 406. (Alternatively, each roller 426, 428 and 430 may take the shape of an hour-glass.) An adjustment device 432 moves the two outer rollers 426, 430 laterally against the vein 406 in one direction and the intermediate roller 428 against the vein 406 in the opposite direction to bend the vein to thereby restrict the blood flow in the vein 406, see FIG. 39B. To increase or restore the blood flow in the vein 406, the adjustment device 432 moves the rollers 426-430 away from the vein 406 to release the vein from the rollers 426-430, see FIG. 39C.

FIGS. 704-40A through 704-44B schematically illustrates modifications of the above embodiment according to FIGS. 704-39A-704-39C. Thus, FIGS. 704-40A and 704-40B show an embodiment similar to that of FIGS. 39A-39C except that the bending members are oval and not rotatable. FIGS. 704-41A and 704-41B show an embodiment similar to that of FIGS. 704-40A and 40B except that the oval bending members are rotatable to release the vein, see FIG. 704-41A, and squeeze the vein, see FIG. 704-41B. FIGS. 704-42A and 704-42B show an embodiment similar to that of FIGS. 704-39A-704-39C except that the intermediate roller has a changeable diameter to release the vein, see FIG. 704-42A, and squeeze the vein, see FIG. 704-42B. FIGS. 704-43A and 704-43B show an embodiment similar to that of FIGS. 704-37A-704-37C except that the rigid elements are replaced by two cylindrical rollers positioned on opposite sides of the vein. Finally, FIGS. 704-44A and 704-44B show an embodiment substantially similar to that of FIGS. 43A and 43B except that the restriction device is curved to form an S-shaped curvature of the vein.

FIG. 704-45 schematically illustrates how any of the above-described embodiments of the male sexual impotence treatment apparatus may be implanted in a patient. Thus, the apparatus comprises an adjustable restriction device 434 extending around the corpus cavernosum of the patient and a motor operated adjustment device 436 for mechanically adjusting the restriction device 434 to squeeze the corpus cavernosum to thereby restrict the blood flow leaving the penis. The motor, not shown, is integrated in the adjustment device 436 and is reversible to operate the adjustment device 436 to release the corpus cavernosum from the restriction device 434. A wireless remote control of the apparatus comprises an external signal transmitter 438 incorporated in a portable remote-control case and an implanted signal receiver 440, which comprises a control unit for controlling the adjustment device 436 in response to a control signal, for example an electromagnetic wave signal, from the transmitter 438. The signal receiver 440 further comprises an energizer unit which transfers energy from the control signal transmitted by the transmitter 438 into electric energy for energy consuming implanted components of the apparatus, such as the motor for operating the adjustment device 436. The electric energy is conducted via an implanted conductor 442 from the signal receiver 440 to the motor. When the patient wishes to achieve erection, he readily uses the portable remote-control case to activate the implanted adjustment device 436 to temporarily adjust the implanted restriction device 434 to restrict the blood flow leaving his penis.

FIG. 704-46 shows an embodiment which is identical to the embodiment of FIG. 704-45, except that the restriction device comprises two restriction members 444 and 446 extending around respective exit veins from the penis. A motor operated adjustment device, not shown, is incorporated in each restriction member 444, 446. There are a number of other conceivable alternative embodiments that give the same result as the above-described embodiments. For example, the microprocessor of the external and implanted, respectively, control unit may be replaced by discrete components. The power amplifier of the external control unit may be omitted if the signals generated by the signal generator are strong enough. Therefore the invention is to be accorded the broadest interpretation of the appended claims to encompass all equivalent structures and assemblies.

### AC: Impotence Treatment

FIG. 705-1 schematically shows an embodiment of the male sexual impotence treatment apparatus having some parts implanted in a patient and other parts located outside the patient's body. Thus, in FIG. 705-1 all parts placed to the right of the patient's skin 2 are implanted and all parts placed to the left of the skin 2 are located outside the patient' s body. The apparatus of FIG. 705-1 comprises an implanted operable restriction device 4, which engages the patient's penile tissue to form a restrictable blood flow passageway for blood flow leaving the penis. The restriction device 4 is capable of performing a reversible function, i.e. to enlarge and reduce the cross-sectional area of the restrictable passageway, whereby the restriction device works as an artificial sphincter. An implanted control unit 6 controls the restriction device 4 via a control line 8 to form an adequate size of the cross-sectional area of the restrictable passageway. An external control unit 10 includes an external source of energy and a wireless remote control transmitting a control signal generated by the external source of energy. The control signal is received by a signal receiver incorporated in the implanted control unit 6, whereby the control unit 6 controls the implanted restriction device 4 in response to the control signal. The implanted control unit 6 also uses energy from the control signal for directly operating the restriction device 4 via a power supply line 12, as the control signal is transmitted.

FIG. 705-2 shows an embodiment identical to that of FIG. 705-1, except that a reversing device in the form of a switch 14 operable by energy also is implanted in the patient for reversing the restriction device 4. The control unit 6 uses the switch 14 to reverse the function performed by the restriction device 4. More precisely, the external control unit 10 releases energy carried by a wireless signal and the implanted control unit 6 transforms the wireless energy into a current for operating the switch 14. When the control unit 6 shifts the polarity of the current the switch 14 reverses the function performed by the restriction device 4.

FIG. 705-3 shows an embodiment identical to that of FIG. 705-1, except that an operation device in the form of a motor 16 also is implanted in the patient. The implanted control unit 6 powers the motor 16 with wireless energy released from the external source of energy of the external control unit 10. The implanted control unit 6 controls the operation of the motor 16 in response to a control signal from the remote control of the external control unit 10.

FIG. 705-4 shows an embodiment identical to that of FIG. 705-1, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to restrict the urine passageway, and hydraulic fluid is pumped by the motor/pump unit 18 back from the restriction device 4 to the reservoir to enlarge the urine passageway. The external control unit 10 releases energy carried by a wireless signal and the implanted control unit 6 transforms the wireless energy into a current, for example a current, for powering the motor/pump unit 18 via an electric power supply line 24. The implanted control unit 6 controls the motor/pump unit 16 and the restriction device 4 via control lines 26 and 27.

FIG. 705-5 shows an embodiment comprising the restriction device 4, hydraulically operated, and the implanted control unit 6, and further comprising a hydraulic fluid reservoir 230, a motor/pump unit 232 and a reversing device in the form of a hydraulic valve shifting device 234, all of which are implanted in the patient. The motor of the motor/pump unit 232 is an electric motor.

FIG. 705-6 shows an embodiment identical to that of FIG. 705-1, except that an accumulator 28 also is implanted in the patient. The control unit 6 stores energy received from the external control unit 10 in the accumulator 28. In response to a control signal from the external control unit 10 the implanted control unit 6 releases energy from the accumulator 28 via a power line 30 for the operation of the restriction device 4.

FIG. 705-7 shows an embodiment comprising the restriction device 4, hydraulically operated, and the implanted control unit 6, and further comprising a source of energy in the form of a battery 32, a hydraulic fluid reservoir 34, a motor/pump unit 36 and a reversing device in the form of a hydraulic valve shifting device 38, all of which are implanted in the patient. The motor of the motor/pump unit 36 is an electric motor. An external control unit 40 includes a wireless remote control transmitting a control signal which is received by the signal receiver incorporated in the implanted control unit 6. In response to a control signal from the external control unit 40 the implanted control unit 6 powers the motor/pump unit 36 with energy from the battery 32, whereby the motor/pump unit 36 distributes hydraulic fluid between the reservoir 34 and the restriction device 4. The control unit 6 controls the shifting device 38 to shift the hydraulic fluid flow direction between one direction in which the fluid is pumped by the motor/pump unit 36 from the reservoir 34 to the restriction device 4 to restrict the passageway, and another opposite direction in which the fluid is pumped by the motor/pump unit 36 back from the restriction device 4 to the reservoir 34 to enlarge the blood flow passageway.

FIG. 705-8 shows an embodiment identical to that of FIG. 705-6, except that a battery 42 is substituted for the accumulator 28, the external control unit 40 of the embodiment of FIG. 705-5 is substituted for the external control unit 10 and an electric motor 44 is implanted in the patient for operating the restriction device 4. In response to a control signal from, the external control unit 40 the implanted control unit 6 powers the motor 44 with energy from the battery 42, whereby the motor 44 operates the restriction device 4.

FIG. 705-9 shows an embodiment identical to that of FIG. 705-8, except that the motor/pump unit 36 of the embodiment of FIG. 705-7 is substituted for the motor 44 and a fluid reservoir 46 also is implanted in the patient. The reservoir 46 is via fluid conduits 48 and 50 connected to the motor/pump unit 36 and restriction device 4, which in this case is hydraulically operated. In response to a control signal from the external control unit 40, the implanted control unit 6 powers the electric motor of the motor/pump unit 36 with energy from the battery 42, whereby the motor/pump unit 36 distributes hydraulic fluid between the fluid reservoir 46 and the restriction device 4.

FIG. 705-10 shows an embodiment identical to that of FIG. 705-8, except that a mechanical reversing device in the form of a gearbox 52 also is implanted in the patient. The implanted control unit 6 controls the gearbox 52 to reverse the function performed by the restriction device 4 (mechanically operated).

FIG. 705-11 shows an embodiment comprising the restriction device 4, the external control unit 10, an implanted source of energy 236 and an implanted switch 238. The switch 238 is operated by wireless energy released from the external source of energy of the external control unit 6 to switch between an off mode, in which the implanted source of energy 236 is not in use, and an on mode, in which the implanted source of energy 236 supplies energy for the operation of the restriction device 4.

FIG. 12 shows an embodiment identical to that of FIG. 705-11, except that also the control unit 6 is implanted, in order to receive a control signal from the wireless remote control of the external control unit 10. The switch 238 is operated by the wireless energy from the external source of energy 10 to switch between an off mode, in which the implanted source of energy 236 and the wireless remote control of the external control unit 10 are not in use, i.e. the control unit 6 is not capable of receiving the control signal, and a standby mode, in which the wireless remote control is permitted to control the internal source of energy 236, via the implanted control unit 6, to supply energy for the operation of the restriction device 4.

FIG. 705-13 shows an embodiment identical to that of FIG. 705-12, except that an energy transforming device for transforming the wireless energy into storable energy is incorporated in the implanted control unit 6 and that the implanted source of energy 236 is of a type that is capable of storing the storable energy. In this case, in response to a control signal-from the external control unit 10, the implanted control unit 6 controls the switch 238′ to switch from an off mode, in which the implanted source of energy 236 is not in use, to an on mode, in which the source of energy 36 supplies energy for the operation of the restriction device 4.

FIG. 705-14 shows an embodiment identical to that of FIG. 705-13, except that an energy storage device 240 also is implanted in the patient for storing the storable energy transformed from the wireless energy by the transforming device of the control unit 6. In this case, the implanted control unit 6 controls the energy storage device 240 to operate the switch 238 to switch between an off mode, in which the implanted source of energy 236 is not in use, and an on mode, in which the implanted source of energy 236 supplies energy for the operation of the restriction device 4.

FIG. 705-15 shows an embodiment identical to that of FIG. 705-13, except that a motor 242 and a mechanical reversing device in the form of a gearbox 244 also are implanted in the patient. The implanted control unit 6 controls the gearbox 244 to reverse the function performed by the restriction device 4 (mechanically operated), i.e. enlarging and restricting the blood flow passageway.

FIG. 705-16 schematically shows conceivable combinations of implanted components of the apparatus for achieving various communication possibilities. Basically, there are the implanted restriction device 4, the implanted control unit 6 and the external control unit 10 including the external source of energy and the wireless remote control. As already described above the remote control transmits a control signal generated by the external source of energy, and the control signal is received by a signal receiver incorporated in the implanted control unit 6, whereby the control unit 6 controls the implanted restriction device 4 in response to the control signal.

A sensor 54 may be implanted in the patient for sensing a physical parameter of the patient, such as the pressure in the stomach. The control unit 6, or alternatively the external control unit 10, may control the restriction device 4 in response to signals from the sensor 54. A transceiver may be combined with the sensor 54 for sending information on the sensed physical parameter to the external control unit 10. The wireless remote control of the external control unit 10 may comprise a signal transmitter or transceiver and the implanted control unit 6 may comprise a signal receiver or transceiver. Alternatively, the wireless remote control of the external control unit 10 may comprise a signal receiver or transceiver and the implanted control unit 6 may comprise a signal transmitter or transceiver. The above transceivers, transmitters and receivers may be used for sending information or data related to the restriction device from inside the patient's body to the outside thereof. The motor 44 may be implanted for operating the restriction device 4 and also the battery 32 may be implanted for powering the motor 44. The battery 32 may be equipped with a transceiver for sending information on the charge condition of the battery. Those skilled in the art will realize that the above various embodiments according to

FIGS. 705-1-705-15 could be combined in many different ways. For example, the energy operated switch 14 could be incorporated in any of the embodiments of FIGS. 705-4, 705-6, 705-8-705-10. The hydraulic shifting device 38 could be incorporated in any of the embodiments of FIGS. 705-4 and 705-9. The gearbox 52 could be incorporated in any of the embodiments of FIGS. 705-1, 705-6 and 705-8.

FIG. 705-17 illustrates how any of the above-described embodiments of the apparatus may be implanted in a patient. Thus, the apparatus comprises-, a restriction device 56 implanted in the patient and engaging the penile tissue 58, and an operation device 60 for operating the restriction device 56. A control device in the form of a wireless remote control comprises an implanted control unit 62, which includes a signal receiver, for controlling the operation device 60, and an external control unit 64 including a signal transmitter for transmitting a control signal to the signal receiver of the implanted control unit 62. The implanted control unit 62 is capable of transforming signal energy from the control signal into electric energy for powering the operation device 60 and for energizing electric energy consuming implanted components of the apparatus. FIG. 705-18 shows the basic parts of a wireless remote control of the apparatus including an electric motor 128 for operating a restriction device, for example of the type illustrated in FIG. 705-17. In this case, the remote control is based on the transmission of electromagnetic wave signals, often of high frequencies in the order of 100 kHz-1 gHz, though, the skin 130 of the patient. In FIG. 705-18, all parts placed to the left of the skin 130 are located outside the patient's body and all parts placed to the right of the skin 130 are implanted. Any suitable remote control system may be used.

An external signal transmitting antenna 132 is to be positioned close to a signal receiving antenna 134 implanted close to the skin 130. As an—alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same restriction as the coil of the receiving antenna 134 but wound with a thick wire that can handle the larger currents that is necessary. The coil of the transmitting antenna 132 is tuned to the same specific high frequency as the coil of the receiving antenna 134.

An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to activate the restriction device to either restrict or enlarge the blood flow passageway. The microprocessor starts a command by applying a high frequency signal on the antenna 132. After a short time, when the signal has energized the implanted parts of the control system, commands are sent to restrict or enlarge the blood flow passageway in predefined steps. The commands are sent as digital packets in the form illustrated below.

| | | | |
|---|---|---|---|
| Start | Command, | Count, | Checksum, |
| pattern, | 8 bits | 8 bits | 8 bits |
| 8 bits | | | |

The commands are sent continuously during a rather long time period {e.g. about 30 seconds or more). When a new restrict or enlarge step is desired the Count byte is increased by one to allow the implanted control unit 138 to decode and understand that another step is demanded by the external control unit 136. If any part of the digital packet is erroneous, its content is simply ignored. Through a line 140, an implanted energizer unit 126 draws energy from the high frequency electromagnetic wave signals received by the receiving antenna 134. The energizer unit 126 stores the energy in a power supply, such as a large capacitor, powers the control unit 138 and powers the electric motor 128 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to operate the restriction device to either restrict or enlarge the blood flow passageway depending on the received command code.

Alternatively, the energy stored in the power supply of the energizer unit may only be used for powering a switch, and the energy for powering the motor 128 may be obtained from another implanted power source of relatively high capacity, for example a battery. In this case the switch is adapted to connect the battery to the control unit 138 in an on mode when the switch is powered by the power supply and to keep the battery disconnected from the control unit in a standby mode when the switch is unpowered;

With reference to FIG. 705-19, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178. The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### AD: Urinary Incontinence

FIG. 706-1 schematically shows a most simple embodiment of the urine incontinence disease apparatus having some parts implanted in a patient and other parts located outside the patient's body. Thus, in FIG. 706-1 all parts placed to the right of the patient's skin 2 are implanted and all parts placed to the left of the skin 2 are located outside the patient's body. The apparatus of FIG. 706-1 comprises an implanted operable restriction device 4, which engages the patient's urethra (or alternatively engages the urine bladder) to form a restricted urine passageway in the urethra. The restriction device 4 is capable of performing a reversible function, i.e. to enlarge and reduce the cross-sectional area of the urine passageway, whereby the restriction device 4 works as an artificial sphincter. An implanted energy transforming device 6 is adapted to supply energy consuming components of the restriction device 4 with energy via a power supply line 12. An external energy transmission device 10 includes a wireless remote control for transmitting a wireless signal, which is received by a signal receiver incorporated in the implanted energy transforming device 6. The implanted energy transforming device 6 transforms energy from the signal into electric energy that is supplied via the power supply line 12. FIG. 706-2 shows an embodiment identical to that of FIG. 706-1, except that a reversing device in the form of an electric switch 14 energy also is implanted in the patient for reversing the restriction device 4. The wireless remote control of the external energy transmission device 10 transmits a wireless signal that carries energy and the implanted energy transforming device 6 transforms the wireless energy into a current for operating the switch 14. When the polarity of the current is shifted by the energy transforming device 6 the switch 14 reverses the function performed by the restriction device 4. FIG. 706-3 shows an embodiment identical to that of FIG. 706-1, except that an operation device in the form of a motor 15 for operating the restriction device 4 also is implanted in the patient. The motor 15 is powered with energy from the energy transforming device 6, as the remote control of the external energy transmission device 10 transmits a wireless signal to the receiver of the energy transforming device 6. FIG. 706-4 shows an embodiment identical to that of FIG. 706-1, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to reduce the cross-sectional area of the urine passageway, and hydraulic fluid is pumped by the motor/pump unit 18 back from the restriction device 4 to the reservoir 20 to enlarge the cross-sectional area. The implanted energy transforming device unit 6 transforms wireless energy into a current, for example a current, for powering the motor/pump unit 18 via an electric power supply line 24.

FIG. 706-5 shows an embodiment comprising the external energy transmission device 10 with its wireless remote control, the restriction device 4, in this case hydraulically operated, and the implanted energy transforming device 6, and further comprising an implanted hydraulic fluid reservoir 30, an implanted motor/pump unit 32 and an implanted reversing device in the form of a hydraulic valve shifting device 34. The motor of the motor/pump unit 32 is an electric motor. In response to a control signal from the wireless remote control of the external energy transmission device 10, the implanted energy transforming device 6 powers the motor/pump unit 32 with energy from the energy carried by the control signal, whereby the motor/pump unit 32 distributes hydraulic fluid between the reservoir 30 and the restriction device 4. The remote control of the energy transmission device 10 controls the shifting device 34 to shift the hydraulic fluid flow direction between one direction in which the fluid is pumped by the motor/pump unit 32 from the reservoir 30 to the restriction device 4 to reduce the cross-sectional area of the urine passageway, and another opposite direction in which the fluid is pumped by the motor/pump unit 32 back from the restriction device 4 to the reservoir 30 to enlarge the cross-sectional area.

FIG. 706-6 shows an embodiment identical to that of FIG. 706-1, except that a control unit 36 controlled by the wireless remote control of the external energy transmission device 10, an accumulator 38 and a capacitor 40 also are implanted in the patient. The control unit 36 stores electric energy received from the energy transforming device 6 in the accumulator 38, which supplies energy to the restriction device 4. In response to a control signal from the wireless remote control of the energy transmission device 10, the control unit 6 either releases electric energy from the accumulator 38 and transfers the released energy via power lines 42 and 44, or directly transfers electric energy from the energy transforming device 6 via a power line 46, the capacitor 40, which stabilizes the electric current, a power line 48 and the power line 44, for the operation of the restriction device 4.

In accordance with one alternative, the capacitor 40 in the embodiment of FIG. 706-6 may be omitted. In accordance with another alternative, the accumulator 38 in this embodiment may be omitted.

FIG. 706-7 shows an embodiment identical to that of FIG. 706-1, except that a battery 50 for supplying energy for the operation of the restriction device 4 and an electric switch 52 for switching the operation of the restriction device 4 also are implanted in the patient. The switch 52 is operated by the energy supplied by the energy transforming device 6 to switch from an off mode, in which the battery 50 is not in use, to an on mode, in which the battery 50 supplies energy for the operation of the restriction device 4.

FIG. 706-8 shows an embodiment identical to that of FIG. 706-7, except that a control unit 36 controllable by the wireless remote control of the external energy transmission device 10 also is implanted in the patient. In this case, the switch 52 is operated by the energy supplied by the energy transforming device 6 to switch from an off mode, in which the wireless remote control is prevented from controlling the control unit 36 and the battery is not in use, to a standby mode, in which the remote control is permitted to control the control unit 36 to release electric energy from the battery 50 for the operation of the restriction device 4. FIG. 706-9 shows an embodiment identical to that of FIG. 706-8, except that an accumulator 38 is substituted for the battery 50 and the implanted components are interconnected differently. In this case, the accumulator 38 stores energy from the energy transforming device 6. In response to a control signal from the wireless remote control of the external energy transmission device 10, the implanted control unit 36 controls the switch 52 to switch from an off mode, in which the accumulator 38 is not in use, to an on mode, in which the accumulator 38 supplies energy for the operation of the restriction device 4.

FIG. 706-10 shows an embodiment identical to that of FIG. 706-9, except that a battery 50 also is implanted in the patient and the implanted components are interconnected differently. In response to a control signal from the wireless remote control of the external energy transmission device 10, the implanted control unit 36 controls the accumulator 38 to deliver energy for operating the switch 52 to switch from an off mode, in which the battery 50 is not in use, to an on mode, in which the battery 50 supplies electric energy for the operation of the restriction device 4. Alternatively, the switch 52 may be operated by energy supplied by the accumulator 38 to switch from an off mode, in which the wireless remote control is prevented from controlling the battery 50 to supply electric energy and is not in use, to a standby mode, in which the wireless remote control is permitted to control the battery 50 to supply electric energy for the operation of the restriction device 4.

FIG. 706-11 shows an embodiment identical to that of FIG. 706-7, except that a motor 15, a mechanical reversing device in the form of a gearbox 54 and a control unit 36 for controlling the gearbox 54 also are implanted in the patient. The implanted control unit 36 controls the gearbox 54 to reverse the function performed by the restriction device 4 (mechanically operated).

FIG. 706-12 shows an embodiment identical to that of FIG. 706-10 except that the implanted components are interconnected differently. Thus, in this case the battery 50 powers the control unit 36 when the accumulator 38, suitably a capacitor, activates the switch 52 to switch to an on mode. When the switch 52 is in its on mode the control unit 36 is permitted to control the battery 50 to supply, or not supply, energy for the operation of the restriction device 4.

FIG. 706-13 schematically shows conceivable combinations of implanted components of the apparatus for achieving various communication options. Basically, there are the implanted restriction device 4, control unit 36 and motor/pump unit 18, and the external energy transmission device 10 including the external wireless remote control. As already described above the wireless remote control transmits a control signal which is received by the implanted control unit 36, which in turn controls the various implanted components of the apparatus.

A sensor 56 may be implanted in the patient for sensing a physical parameter of the patient, such as the pressure in the urine passageway. The implanted control unit 36, or alternatively the external wireless remote control of the energy transmission device 10, may control the restriction device 4 in response to signals from the sensor 56. A transceiver may be combined with the sensor 56 for sending information on the sensed physical parameter to the external wireless remote control. The wireless remote control may comprise a signal transmitter or transceiver and the implanted control unit 36 may comprise a signal receiver or transceiver. Alternatively, the wireless remote control may comprise a signal receiver or transceiver and the implanted control unit 36 may comprise a signal transmitter or transceiver. The above transceivers, transmitters and receivers may be used for sending information or data related to the restriction device 4 from inside the patient's body to the outside thereof. Where the motor/pump unit 18 and battery 50 for powering the motor/pump unit 18 are implanted, the battery 50 may be equipped with a transceiver for sending information on the condition of the battery 50. Those skilled in the art will realize that the above various embodiments according to FIGS. 706-1-706-13 could be combined in many different ways. For example, the energy operated switch 14 could be incorporated in any of the embodiments of FIGS. 706-3, 706-6-706-12, the hydraulic shifting device 34 could be incorporated in the embodiment of FIG. 706-4, and the gearbox 54 could be incorporated in the embodiment of FIG. 706-3.

FIG. 706-14 shows an energy transforming device in the form of an electrical junction element 58 for use in any of the above embodiments according to FIGS. 706-1-706-13. The element 58 is a flat p-n junction element comprising a p-type semiconductor layer 60 and an n-type semiconductor layer 62 sandwiched together. A light bulb 64 is electrically connected to opposite sides of the element 58 to illustrate how the generated current is obtained. The output of current from such a p-n junction element 58 is correlated to the temperature. See the formula below.

$$I = 10 ( exp . ( qV / kT ) - 1 )$$

where
I is the external current flow,
I0 is the reverse saturation current, q is the fundamental electronic charge of 1.602×10-19 coulombs, V is the applied voltage, k is the Boltzmann constant, and
T is the absolute temperature.

Under large negative applied voltage (reverse bias), the exponential term becomes negligible compared to 1.0, and I is approximately −10. 10 is strongly dependent on the temperature of the junction and hence on the intrinsic-carrier concentration. 10 is larger for materials with smaller bandgaps than for those with larger bandgaps. The rectifier action of the diode—that is, its restriction of current flow to only one direction—is in this particular embodiment the key to the operation of the p-n junction element 58. An alternative way to design a p-n junction element is to deposit a thin layer of semiconductor onto a supporting material which does not absorb the kind of energy utilized in the respective embodiments. For use with wirelessly transmitted energy in terms of light waves, glass could be a suitable material. Various materials may be used in the semiconductor layers such as but not limited to cadmium telluride, copper-indium-diselenide and silicon. It is also possible to use a multilayer structure with several layers of p and n-type materials to improve efficiency. The electric energy generated by the p-n junction element 58 could be of the same type as generated by solar cells, in which the negative and positive fields create a direct current. Alternatively, the negative and positive semiconductor layers may change polarity following the transmitted waves, thereby generating an alternating current. The p-n junction element 58 is designed to make it suited for implantation. Thus, all the external surfaces of the element 58 in contact with the human body are made of a biocompatible material. The p-n junction semiconductors are designed to operate optimally at a body temperature of 37° C. because the current output, which should be more than 1 μA, is significantly depending on temperature as shown above. Since both the skin and subcutis absorb energy, the relation between the sensitivity or working area of the element 58 and the intensity or strength of the wireless energy transmission is considered. The p-n junction element 58 preferably is designed flat and small. Alternatively, if the element 58 is made in larger sizes it should be flexible, in order to adapt to the patient's body movements. The volume of the element 58 should be kept less than 2000 cm3.

FIG. 706-15 generally illustrates how any of the above-described embodiments of the urine incontinence disease treatment apparatus may be implanted in a patient. Thus, a restriction device 4 implanted in a patient engages the urethra 66 to form an artificial sphincter around the urine passageway in the urethra 66. An implanted operation device 68, such as an electric motor or a motor/pump assembly, operates the restriction device 4 through a transmission member 70, such as a mechanical transmission cord or a fluid tube. An energy transforming device in the form of an element 6 having a positive region and a negative region, as described above in more detail, is placed underneath the skin of the patient.

Wireless energy carried by a signal transmitted by a wireless remote control of an external energy transmission device 10 at least partly penetrates the patient's skin and hits the element 6. The energy thus hitting the element 6 is transformed into energy of a different form that is suited for powering the operation device 68. For example, where the operation device 68 is an electric motor the element 6 comprises an electric p-n junction element that transforms the wireless energy into an electric current for powering the electric motor. Where the operation device 68 comprises a pump, the element 6 may transform the wireless energy into kinetic energy for powering the pump.

The transformed energy may be utilized for directly operating the restriction device 4 or, where the restriction device 4 is electrically operated, for storage in a capacitor and/or an accumulator for later or parallel use. Preferably (but not necessarily) the element 6 is controlled by a microprocessor. The wireless remote control of the external energy transmission device 10 is used to control the utilization of the transmitted energy and any function or command to/from the implanted restriction device 4.

FIG. 706-16 shows the basic parts of a wireless remote control of the apparatus including an electric motor 128 for operating a restriction member, for example of the type illustrated in FIG. 706-15. In this case, the remote control is based on the transmission of electromagnetic wave signals, often of high frequencies in the order of 100 kHz-1 gHz, through the skin 130 of the patient. In FIG. 706-15, all parts placed to the left of the skin 130 are located outside the patient's body and all parts placed to the right of the skin 130 are implanted. Any suitable remote control system may be used.

An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134.

An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either contract or enlarge the restriction device. The microprocessor starts a command by applying a high frequency signal on the antenna 132. After a short time, when the signal has energized the implanted parts of the control system, commands are sent to contract or enlarge the restriction device in predefined steps. The commands are sent as digital packets in the form illustrated below.

The commands are sent continuously during a rather long time period (e.g. about 30 seconds or more). When a new contract or enlarge step is desired the Count byte is increased by one to allow the implanted control unit 138 to decode and understand that another step is demanded by the external control unit 136. If any part of the digital packet is erroneous, its content is simply ignored.

Through a line 140, an implanted energizer unit 126 draws energy from the high frequency electromagnetic wave signals received by the receiving antenna 134. The energizer unit 126 stores the energy in an energy storage device, such as a large capacitor, powers the control unit 138 and powers the electric motor 128 via a line 142. The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to either contract or enlarge the restriction device depending on the received command code.

Alternatively, the energy stored in the energy storage device of the energizer unit may only be used for powering a switch, and the energy for powering the motor 128 may be obtained from another implanted energy source of relatively high capacity, for example a battery. In this case the switch is adapted to connect said battery to the control unit 138 in an on mode when said switch is powered by the energy storage device and to keep the battery disconnected from the control unit in a standby mode when the switch is not powered.

With reference to FIG. 706-17, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148.

The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation.

A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178.

The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### AE: Hydraulic Urinary Incontinence Treatment Apparatus

FIGS. 707-1A-D is a block diagram of four different embodiments of the urinary incontinence treatment apparatus according to the invention. FIG. 707-1A shows an elongated restriction member in the form of a band 2 forming a loop which defines a restriction opening. The band 2 provides a restricted urine passageway in the urethra when applied around the latter. FIG. 707-1A further shows a separate reservoir 4, a one way pump 6 and an alternate valve 8. FIG. 707-1B shows the band 2 and a fluid supply reservoir 10. FIG. 707-1C shows the band 2, a two way pump 12 and the reservoir 4. FIG. 707-1D shows a servo system with a first closed system controlling a second system. The servo system comprises the fluid supply reservoir 10 and a servo reservoir 14. The servo reservoir 14 controls a larger adjustable reservoir 16 which in connection with the band 2 applied around the urethra varies the volume of a cavity in the band, which in turn varies the restricted urine passageway in the urethra. Such a band 2 forming the restriction opening 3 is illustrated schematically in FIG. 707-8. The band 2 comprises an adjustment device having an expandable/contractable cavity 5 which is expanded or contracted by supplying hydraulic fluid (e.g. from reservoir 4, 6, 10, or 16), and the band 2 may be sutured in place, illustrated schematically at 7 in FIG. 707-8.

FIGS. 707-2A-D are cross-sectional views of a pump mechanism adapted to pump fluid in both directions only by mechanically pushing a separate sealing wall portion 18 in one direction. FIG. 707-2A shows a piston 20 pushed forwards against a spring 22 towards the wall portion 18 and located in a pump housing 24 conducting fluid from a right upper fluid passage 26 of the housing 24 to a left fluid passage 28 of the housing 24. A main valve 30 is open and a nonreturn valve 32 is closed. FIG. 707-2B illustrates the first pump movement in which the piston 20 has moved forwards and reaches the wall portion 18.

FIG. 707-2C illustrates how the piston 20 moves backwards by the action of the spring 22. The main valve 30 is now closed and the nonreturn valve 32 is open for fluid from the right upper passage 26. FIG. 707-1D illustrates how the piston 20 is moved further downwards from its position according to FIG. 707-2B while pushing the wall portion 18 downwardly against a second spring 34 that is stronger than spring 22, whereby fluid escapes from a right lower fluid passage 36. When moving the piston 20 backwardly from the position according to FIG. 707-2D, fluid enters the left fluid passage 28 and a valve 38 in the lower right fluid passage 36 closes.

FIG. 707-3 is a cross-sectional view of a reservoir 40 defining a chamber 42, the volume of which is variable by an operation device in the form of a remote controlled electric motor 44, in accordance with FIG. 707-1B or 707-1D. The reservoir 40 and the motor 44 are placed in a housing 46. The chamber 42 is varied by moving a large wall 48. The wall 48 is secured to a nut 50, which is threaded on a rotatable spindle 52. The spindle 52 is rotated by the motor 44 via an angular gearing, which comprises two conical gear wheels 54 and 56 in mesh with each other. The motor 44 is powered by a battery 58 placed in the housing 46. An signal receiver 60 for controlling the motor 44 is also placed in the housing 46. Alternatively, the battery 58 and the signal receiver 60 may be mounted in a separate place. The motor 44 may also be powered by energy transferred from transmitted signals.

FIG. 707-4 is a cross-sectional view of a reservoir 62 defining a chamber 64, the volume of which is variable and is controlled by manual manipulation. A gable wall portion 66 of an open ended inner cylindrical housing 68 is adapted to be pushed downwards to fit in a desired locking groove 70 of a plurality of locking grooves 70 on the mantle wall of the cylindrical housing 68, to reduce the volume of the chamber 64. The inner cylindrical housing 68 is suspended by springs 72 and is telescopically applied on an outer cylindrical housing 74. When pushing the inner cylindrical housing 68 it moves downwards relative to the outer cylindrical housing 74 causing the gable wall portion 66 to release from the locking groove 70 and move upwards relative to the inner cylindrical housing 68. When the inner housing 68 is moved upwardly by the action of the springs 72 the volume of the chamber 64 is increased. FIGS. 707-5A and 707-5B show a servo means comprising a main ring-shaped fluid reservoir 76 defining a chamber 78, the volume of which is variable. Centrally positioned in the main ring-shaped reservoir 76 there is a servo fluid reservoir 80 defining a chamber 82, the volume of which is variable. The chamber 82 of the servo reservoir 80 is substantially smaller than the chamber 78 of the main reservoir 76. The two reservoirs 76 and 80 are situated between two opposite separate walls 84 and 86, and are secured thereto. When changing the amount of fluid in the servo reservoir 80, the two opposite walls 84, 86 are moved towards or away from each other, whereby the volume of the chamber 78 of the main reservoir 76 is changed.

FIG. 707-6 shows the basic parts of a remote control system of the apparatus including the electric motor 44 of the embodiment shown in FIG. 707-3. In this case, the remote control system is based on the transmission of electromagnetic wave signals, often of high frequencies in the order of 100 kHz-1 gHz, through the skin 130 of the patient. In FIG. 707-6, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same size as the coil of the receiving antenna 134 but wound with a thick wire that can handle the larger currents that is necessary. The coil of the transmitting antenna 132 is tuned to the same specific high frequency as the coil of the receiving antenna 134. An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to either increase or decrease the size of the restriction opening defined by the loop of the restriction member 2. The microprocessor starts a command by applying a high frequency signal on the antenna 132. After a short time, when the signal has energized the implanted parts of the control system, commands are sent to increase or decrease the size of the restriction opening of the restriction member 2 in predefined steps. The commands are sent as digital packets in the form illustrated below.
Start pattern, Command, Count, Checksum,
8 bits 8 bits 8 bits 8 bits

The commands are sent continuously during a rather long time period (e.g. about 30 seconds or more). When a new increase or decrease step is desired the Count byte is increased by one to allow the implanted control unit 138 to decode and understand that another step is demanded by the external control unit 136. If any part of the digital packet is erroneous, its content is simply ignored.

Through a line 140, an implanted energizer unit 126 draws energy from the high frequency electromagnetic wave signal received by the receiving antenna 134. The energizer unit 126 stores the energy in a power supply, such as a large capacitor, powers the control unit 138 and powers the electric motor 44 via a line 142.

The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 44 to either increase or decrease the size of the restriction opening of the restriction member 2 depending on the received command code.

Alternatively, the energy stored in the power supply of the energizer unit may only be used for powering a switch, and the energy for powering the motor 44 may be obtained from another implanted power source of relatively high capacity, for example a battery. In this case the switch is adapted to connect the battery to the control unit 138 in an “on” mode when the switch is powered by the power supply and to keep the battery disconnected from the control unit in a “standby” mode when said switch is unpowered.

With reference to FIG. 707-7, the remote control system schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the urinary incontinence apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 44 via an H-bridge 178 comprising transistors 180, 182, 184 and 186. The motor 44 can be driven in two opposite directions by the H-bridge 178. The microprocessor 176 also monitors the amount of stored energy in the storage capacitor 158. Before sending signals to activate the motor 44, the microprocessor 176 checks whether the energy stored in the storage capacitor 158 is enough. If the stored energy is not enough to perform the requested operation, the microprocessor 176 waits for the received signals to charge the storage capacitor 158 before activating the motor 44. FIGS. 707-9A and 707-9B show an embodiment of the apparatus comprising a restriction device 202 having an elongated flexible restriction member 204, such as a belt, a cord or the like. The flexible member 204 extends in a loop around the urethra. (Alternatively, the flexible member 204 may comprise two separate parts on opposite sides of the urethra.) One portion 204A of member 204 is attached to a frame 208 and another portion 204B of member 204 opposite portion 204A in the loop of the flexible member 204 is connected to an adjustment device 210, which is fixed to the frame 208. The adjustment device 210 pulls the flexible member 204 away from portion 204A to squeeze the urethra between two opposite lengths of the flexible member 204 to thereby restrict the urine passageway, see FIG. 96A, and releases the urethra from the flexible member 204 to thereby increase the urine passageway, see FIG. 707-9B.

FIGS. 707-10A and 707-10B show an embodiment of the apparatus comprising a restriction device 212 having two plate or bar elements 214 on opposite sides of the urethra 206. An adjustment device 216 moves the elements 212 in parallel towards each other to squeeze the urethra 206 between the elements 212 to thereby restrict the urine passageway, see FIG. 707-10A, and moves the elements 212 away from each other to enlarge the urine passageway, see FIG. 707-10B.

FIG. 707-11 shows an embodiment of the apparatus comprising a restriction device 218 having two articulated clamping elements 220 positioned on opposite sides of the urethra 206. An adjustment device 222 moves the clamping elements 220 toward each other to clamp the urethra 206 between the clamping elements 220 to thereby restrict the urine passageway, and moves the clamping elements 220 away from each other to release the urethra 206 from the clamping elements 220 to thereby enlarge the urine passageway.

FIGS. 707-12A, 707-12B and 707-12C show an embodiment of the apparatus comprising a restriction device 224 having three bending members in the form of cylindrical rollers 226, 228 and 230 displaced relative one another in a row along the urethra 206 and positioned alternately on opposite sides of the urethra 206. (Alternatively, each roller 226, 228 and 230 may take the shape of an hour-glass.) An adjustment device 232 moves the two outer rollers 226, 230 laterally against the urethra 206 in one direction and the intermediate roller 228 against the urethra 206 in the opposite direction to bend the urethra to thereby restrict the urine passageway, see FIG. 12B. To release the urethra from the rollers 226-230, the adjustment device 232 moves the rollers 226-230 away from the urethra 206, see FIG. 12C.

FIGS. 707-13A through 1707-7B schematically illustrates modifications of the above embodiment according to FIGS. 707-12A-707-12C. Thus, FIGS. 707-13A and 707-13B show an embodiment similar to that of FIGS. 707-12A-707-12C. except that the bending members are oval and not rotatable. FIGS. 707-14A and 707-14B show an embodiment similar to that of FIGS. 707-13A and 707-13B except that the oval bending members are rotatable to squeeze the urethra, see FIG. 707-14B, and to release the urethra, see FIG. 707-14A. FIGS. 707-15A and 707-15B show an embodiment similar to that of FIGS. 707-12A—707-12C except that the intermediate roller has a changeable diameter to squeeze the urethra, see FIG. 707-15B, and to release the urethra, see FIG. 707-15A. FIGS. 707-16A and 707-16B show an embodiment similar to that of FIGS. 707-10A-707-10C except that the elements are replaced by two cylindrical rollers positioned on opposite sides of the urethra. Finally, FIGS. 707-17A and 707-17B show an embodiment substantially similar to that of FIGS. 707-16A and 707-16B except that the restriction device is turned 90° to form an S-shaped curvature of the urethra.

FIG. 707-18 shows an example of a hydraulic restriction device 234 for use in accordance with the invention. The restriction device 234 comprises an elongated restriction member 236 having an inflatable cavity 238. A tube 240 connects the cavity 238 to a hydraulic fluid reservoir, not shown. The restriction member 236 may be wrapped around the urethra. Alternatively, two restriction members 236 may-be used. FIG. 19 schematically illustrates how any of the above-described embodiments of the urinary incontinence treatment apparatus can be implanted in a patient. Thus, an implanted adjustable hydraulic restriction device 234 extends almost completely around the urethra 242 and the urethra J\hat{ }tf—to be capable of squeezing the urethra 242 as a single unit. An adjustment device in the form of an inflatable cavity in the restriction device 234 is adapted to adjust the restriction device 234 so that the urine passageway is restricted. An implanted assembly 246 includes a hydraulic fluid reservoir and an operation device (which may include a pump) for distributing hydraulic fluid between the reservoir and the inflatable/contractible cavity of the restriction device 234 via a fluid conduit 240. A wireless remote control of the apparatus comprises an external signal transmitter 248, which may comprise a hand-held unit, and an implanted signal receiver, which is incorporated in the implanted assembly 246, includes a control unit for controlling the restriction device 234 in response to a control signal from the external transmitter. The signal receiver of the assembly 246 further includes an energizer unit which transforms energy from the control signal transmitted by the external transmitter into electric energy for energy consuming implanted components of the apparatus. A pressure sensor 250 is implanted for sensing the pressure on the restriction device 234. The control unit of the assembly 246 controls the restriction device 234 to release the urethra 242 in response to the pressure sensor 250 sensing an abnormal high pressure. There are a number of conceivable alternative embodiments that give the same result as the above-described embodiments. For example, the microprocessor of the external and implanted, respectively, control units may be replaced by discrete components. The power amplifier of the external control unit may be omitted if the signals generated by the signal generator are strong enough. Therefore, the invention is to be accorded the broadest interpretation of the appended claims to encompass all equivalent structures and assemblies.

### AF: Urinary Incontinence Treatment Apparatus

FIGS. 708-1-708-3 show a preferred embodiment of the urinary incontinence treatment apparatus comprising a restriction device having an elongated restriction member in the form of a circular resilient core 2 with two overlapping end portions 4, 6. The core 2 defines a substantially circular restriction opening and is enclosed in an elastic soft hose 8 except at a releasable and lockable joint 10 of the core 2, which when released enables application of the core 2 with its hose 8 around a tissue of a patient, such as the urethra or urine bladder. The materials of all of these elements are biocompatible so that the patient's body will not reject them. A mechanical adjustment device 12 for mechanically adjusting the longitudinal extension of the core 2 to change the size of the restriction opening comprises a drive wheel 14 in frictional engagement with the overlapping end portions 4, 6 of the core 2. The drive wheel 14 is journalled on a holder 16 placed in the hose 8 and provided with two counter pressure rollers 18, 20 pressing the respective end portions 4, 6 of the core 2 against the drive wheel 14 to increase the frictional engagement there between. An electric motor 22 is connected to the drive wheel 14 via a long flexible drive shaft 24 and is moulded together with a remote controlled power supply unit 26 in a body 28 of silicone rubber. The length of the flexible drive shaft 34 is selected so that the body 28 can be placed in a desired position in the patient's body, suitably in the abdomen.

When the patient doesn't want to relieve himself (urinate), he controls the power supply unit 26 to power the electric motor 22 to turn the drive wheel 14 in one direction to reduce the diameter of the core 2, so that the tissue is squeezed and the urine passageway is restricted. When the patient wishes to urinate, he controls the power supply unit 26 to power the electric motor 22 to turn the drive wheel 14 in the opposite direction to increase the diameter of the core 2, so that the urine passageway is released and opens.

Alternatively, a rack gear may be formed on one of the end portions 4, 6 of the core 2 and the drive wheel 14 may be replaced by a drive gear wheel connected to the other end portion of the core 2 and in mesh with the rack gear.

FIG. 708-4 shows an embodiment which is identical to the embodiment of FIGS. 708-1-708-3, except that the motor 22 is encapsulated in a lateral protrusion 30 of the hose 8 so that it is fixed to the core 2 and has a short drive shaft 32 onto which the drive wheel 14 is mounted, and that the motor 22 is positioned relative to the circular core 2 such that the drive shaft 32 extends radially thereto.

FIG. 708-5 shows an embodiment which likewise is identical to the embodiment of FIGS. 1-3, except that the motor 22 is encapsulated in the hose 8 so that it is fixed to the core 2 and has a short drive shaft 32, and that the motor 22 is positioned relative to the core 2 such that the drive, shaft 32 extends substantially tangentially to the circular core 2. There is an angular gearing 34 connecting the drive shaft 32 to the drive wheel 14.

FIG. 708-6 shows a suitable-arrangement for the motor 22 in the embodiment of FIG. 708-5, comprising a first clamping member 36 secured to one end portion of the core 2 and a second clamping member 38 secured to the other end portion 6 of the core 2. The motor 22 is secured to the first clamping member 36 and is operatively connected to a worm 40 via a gear transmission 42. The worm 40 is journalled at its opposite ends on holders 44 and 46, which are rigidly secured to the clamping member 36 and the motor 22, respectively. The second clamping member 38 has a pinion in mesh with the worm 40. When the motor 22 is powered the worm 40 rotates and pulls the end portion 6. of the core 2 in one or the opposite longitudinal direction, so that the diameter of the substantially circular core 2 is either increased or decreased. FIG. 708-7 shows an embodiment in which the elongated restriction member comprises a core 48 and a helical spring 50. A spring contracting means in the form of a flexible pulling member 52, i.e. a string, wire or cable, is connected to the core 48 at one end thereof and extends through the helical spring 50. A hydraulic motor in the form of a cylinder/piston unit 54 is adapted—to pull the flexible pulling member 52 to contract the helical spring 50 against an arresting member 56, which is fixed relative to the core 48. A tube 58 hinged to the arresting member 56 extends between the cylinder/piston unit 54 and the arresting member 56, the flexible pulling member 52 running through the tube 58 and being connected to the piston of the cylinder/piston unit 54.

FIG. 708-8 shows a similar embodiment in which a hydraulic transmission conduit 59 is provided between two piston-cylinder assemblies 54, for use as the hydraulic motor/device in FIG. 708-7.

FIG. 708-9 shows an embodiment in which the restriction member comprises two elongated helical springs 60 and 62 having free ends, and a body 64 to which the springs 60, 62 are non-rotatably secured at their opposite ends. The body 64 comprises two separate parts secured to opposite end portions of the enclosing elastic hose 8 and is designed with a releasable and lockable joint between the separate parts. An adjustment device in the form of a drive shaft 66 has two opposite end portions connected to the helical springs 60, 62, respectively, at their free ends. The coils of the springs 60, 62 form left and right hand helices, respectively. A motor 68 is adapted to rotate the drive shaft 66 in one direction to enlarge the coils of the helical springs 60, 62 to longitudinally contract the springs 60, 62 and to rotate the drive shaft 66 in the opposite direction to reduce the size of the coils of the springs 60, 62 to longitudinally extend the springs 60, 62. Thus, the elongated helical springs 60, 62 defines a restriction opening, the size of which is increased when the springs 60, 62 are extended and decreased when the springs 60, 62 are contracted. FIG. 708-10 shows an embodiment according to the aspect which is identical to the embodiment of FIG. 708-9, except that the adjustment device comprises a gearing having an input shaft 72 and two opposite aligned output shafts 74 and 76 connected to the helical springs 60 and 62, respectively, at their free ends. The input shaft 72 is connected to the output shafts 74, 76 such that they rotate at opposite directions upon rotation of the input shaft 72. The coils of the springs 60, 62 form the same helices.

FIGS. 708-11-708-14 show an embodiment of the device in which a hydraulic motor comprises two interconnected cylinders 78 and 80 and two pistons 82 and 84 in the respective cylinders 78/80. The cylinders 78, 80 have a common fluid supply inlet member 86, which together with the cylinders 78, 80 takes the shape of an Y-pipe. The restriction member comprises an elongated resilient arcuate core 88. The adjustment device comprises two bars 90 and 92 secured to opposite ends of the core 88 and connected to the pistons 82 and 84, respectively. The core 88 defines a restriction opening and is provided with a releasable and lock-able joint 94 (FIG. 708-13) to permit application of the core 88 around the tissue. A soft elastic hose 96 except at the joint 94 and the inlet member 86 encloses the core 88 and the cylinders 90, 92. The hose 96 has an outer tubular wall 98 and a central coaxial inner tubular wall 100, which is fixed to the outer wall 98 by spoke members 102′ (FIG. 708-14). The core 88 is loosely fit in the inner tubular wall 100. By supplying fluid to or withdrawing fluid from the inlet 86 the pistons 82 and 84 will move towards or from each other, so that the restriction opening defined by the core 88 is changed by the longitudinal displacement of the bars 90, 92.

FIGS. 708-15-708-17 show an embodiment which is identical to the embodiment of FIGS. 708-11-708-14, except that the adjustment device comprises an elongated voltage-responsive element 104 secured to the opposite ends of the core 88, so that the core 88 and the element 104 form the restriction member. The element 104 is capable of bending inwardly into a bow in response to a voltage applied across the element 104. The radius of curvature of the bow is adjustable by changing the level of. the voltage applied to element 104.

FIG. 708-18 shows an embodiment comprising a loop forming means in the form of a substantially rigid outer circular element 106 with a releasable and lockable joint 108. In this embodiment the restriction member comprises an elastic inner circular element 110 formed by the innermost wall portion of an elastic hose 112 extending along the outer element 106. The inner circular element 110 is disposed concentrically within the outer circular element 106. The adjustment device comprises a plurality of threads 114 secured to the elastic inner element 110 along the circumference thereof and running from the inner element 110 via guide members 116 attached to the outer element 106. By pulling all the threads 114 the inner elastic element 110 is pulled under expansion radially outwardly towards the outer element 106.

FIG. 708-19 shows an embodiment which is identical to the embodiment of FIG. 708-9, except that it comprises a loop forming means in the form of a substantially rigid outer circular element 118 supporting the helical springs 60, 62, and a soft elastic inner wall 120 extending along the springs 60, 62. When the motor 68 rotates the helical springs 60, 62 in a direction that enlarges the coils of the springs 60, 62, the coils are forced by the rigid outer element 118 to expand radially inwardly thereby reducing the size of the restriction opening formed by the circumferential confinement surface of the restriction member (springs 60, 62 and body 64).

FIG. 708-20 shows an embodiment in which a restriction member comprises a plurality of arcuate lamellae 122 arranged like the conventional adjustable aperture mechanism of a camera. The adjustment device, not shown, “is conventional and is operated by a motor 124 to adjust the lamellae 122 to change the size of an restriction opening defined by the lamellae 122.

FIGS. 708-21-708-23 show an embodiment in which a restriction member comprises two semi-circular elements 126 and 128 which are hinged together such that the semi-circular elements 126, 128 are swingable relative to each other between a fully open state in which they substantially form a circle, illustrated in FIG. 708-22 and an angular state, in which the size of the restriction opening defined by the semi-circular elements 126, 128 is reduced, illustrated in FIG. 708-23. The adjustment device, not shown, is conventional and is operated by a motor 130 to swing the semi-circular elements 126, 128 relative to each other.

FIGS. 708-24-708-27 show an embodiment in which a restriction member comprises an elastic belt 130 forming a circle and having a substantially oval cross-section. The restriction member 130 is provided with a releasable and lockable joint 132. An elastic double walled hose 134 encloses the belt 130 except at the joint 132. The adjustment device, not shown, is conventional and is operated by a motor 136 to turn the belt 130 around the longitudinal extension thereof between a fully open state, in which the inner broader side of the belt 130 forms a substantially cylindrical surface, illustrated in FIG. 708-26, and a reduced open state, in which the inner broader side of the belt 130 forms a substantially conical surface, illustrated in FIG. 708-27.

FIG. 708-28 schematically illustrates a cushion arrangement for protecting the tissue, comprising a plurality of cushions 138 disposed in series along a substantially circular holding member 140. This cushion arrangement may be utilized in any of the above-described embodiments of the invention.

FIGS. 708-29A-D provide a block diagram of four different hydraulic transmission configurations. FIG. 708-29A shows an adjustment device 202, a separate reservoir 204, a one way pump 206 and an alternate valve 208. FIG. 708-29B shows the adjustment device 202 and an adjustable reservoir 210. FIG. 708-29C shows the adjustment device 202, a two-way pump 212 and the reservoir 204. FIG. 708-29D shows a reverse servo system with a first closed system controlling a second system. The servo system comprises an adjustable servo reservoir 210 and a passive adjustable fluid supply reservoir 214. Any of the reservoirs can be the active reservoir, either the servo reservoir 210 or the fluid supply reservoir 214. The reservoir 214 controls a larger adjustable reservoir 216, which is used for the operation of the adjustment device 202 for changing the restriction opening of the restriction member. FIGS. 708-30A-D are cross-sectional views of a pump mechanism adapted to pump fluid in both directions only by mechanically pushing a separate sealing wall portion 218 in one direction. FIG. 708-30A shows a piston 220 pushed forwards against a spring 222 towards the wall portion 218 and located in a pump housing 224 conducting fluid from a right upper fluid passage 226 of the housing 224 to a left fluid passage 228 of the housing 224. A main valve 230 is open and a nonreturn valve 232 is closed. FIG. 708-30B illustrates the first pump movement in which the piston 220 has moved forwards and reaches the wall portion 218. FIG. 708-30C illustrates how the piston 220 moves backwards by the action of the spring 222. The main valve 230 is now closed and the nonreturn valve 232 is open for fluid from the right upper passage 226. FIG. 708-30D illustrates how the piston 220 is moved further downwards from its position according to FIG. 708-30B while pushing the wall portion 218 downwards against a second spring 234 that is stronger than spring 222, so that fluid escapes from a right lower fluid passage 236. When moving the piston 220 backwards from the position of FIG. 708-30D, fluid enters the left fluid passage 228 and a valve 238 in the lower right fluid passage 236-closes.

FIG. 708-31 is a cross-sectional view of a reservoir 240 defining a chamber 242, the size of which is variable and is controlled by a remote controlled motor 244, in accordance with FIG. 708-29B or 708-29D. The reservoir 240 and the motor 244 are placed in a housing 246. The chamber 242 is varied by moving a large wall 248. The wall 248 is secured to a nut 250, which is threaded on a rotatable spindle 252. The spindle 252 is rotated by the motor 244 via an angular gearing, which comprises two conical gear wheels 254 and 256 in mesh with each other. The motor 244 is powered by a battery 258 placed in the housing 246. A signal receiver 260 for controlling the motor 244 is also placed in the housing 246. Alternatively, the battery 258 and the signal receiver 260 may be mounted in a separate place. The signal receiver may comprise any known or conventional device which is capable of receiving a control signal and then operating the motor 244.

FIG. 708-32 is a cross-sectional view of a reservoir 262 defining a chamber 264, the size of which is variable and is controlled by manual manipulation. A gable wall portion 266 of an open ended inner cylindrical housing 68 is adapted to be pushed downwards to fit in a desired locking groove 270 of a plurality of locking grooves 270 on the mantle wall of the cylindrical housing 268, to reduce the size of the chamber 64. The inner cylindrical housing 268 is suspended by springs 272 and is telescopically applied on an outer cylindrical housing 274. When pushing the inner cylindrical housing 268 it moves downwards relative to the outer cylindrical housing 274 causing the gable wall portion 266 to release from the locking groove 270 and move upwards relative to the inner cylindrical housing 268. When the inner housing 268 is moved upwardly by the action of the springs 272 the size of the chamber 264 is increased.

FIGS. 708-33A and 708-33B show a servo means comprising a main ring-shaped fluid reservoir 276 defining a chamber 278, the size of which is variable. Centrally positioned in the main ring-shaped reservoir 276 there is a servo fluid reservoir 280 defining a chamber 282, the size of which is variable. The chamber 282 of the servo reservoir 280 is significantly smaller than the chamber 278 of the main reservoir 276. The two reservoirs 276 and 280 are situated between two opposite separate-walls 284 and 286, and are secured thereto. When changing the amount of fluid in the servo reservoir 280, the two opposite walls 284, 286 are moved towards or away from each other, whereby the size of the chamber 278 of the main reservoir 276 is changed.

FIG. 708-34 shows the basic parts of a remote control system of the apparatus including a motor, for instance the electric motor 22. In this case, the remote control system is based on the transmission of an electromagnetic wave signal, often of a high frequency in the order of 100 kHz-1 gHz, through the skin 330 of the patient. In FIG. 708-34, all parts placed to the left of the skin 330 are located outside the patient's body and all parts placed to the right of the skin 330 are implanted in the patient's body. An external signal transmitting antenna 332 is to be positioned close to a signal receiving antenna 334 implanted in the patient's body close to the skin 330. As an alternative, the receiving antenna 334 may be placed for example inside the abdomen of the patient. The receiving antenna 334 comprises a coil, approximately 1-100 mm, preferably 25 mm in diameter wound with a very thin wire and tuned with a capacitor to a specific high frequency. A small coil is chosen if it is to be implanted under the skin of the patient and a large coil is chosen if it is to be implanted in the abdomen of the patient. The transmitting antenna 332 comprises a coil having about the same size as the coil of the receiving antenna 334 but wound with a thick wire that can handle the larger currents that is necessary. The coil of the transmitting antenna 332 is tuned to the same specific high frequency as the coil of the receiving antenna 334. An external control unit 336 comprises a microprocessor, a high frequency electromagnetic signal generator and a power amplifier. The microprocessor of the control unit 336 is adapted to switch on/off the generator and to modulate signals generated by the generator to send digital information via the power amplifier and the antennas 332, 334 to an implanted control unit 338. To avoid that accidental random high frequency fields trigger control commands, digital signal codes are used. A keypad placed on the external control unit 336 is connected to the microprocessor thereof. The keypad is used to order the microprocessor to send a digital signal to either increase or decrease the size of the restriction opening defined by the loop of the restriction member (e.g. as described above). The microprocessor starts a command by applying a high frequency signal on the antenna 332. After a short time, when the signal has energized the implanted parts of the control system, commands are sent to increase or decrease the size of the restriction opening of the restriction member in predefined steps. The commands are sent as digital packets in the form illustrated below.
Start pattern, Command, Count, Checksum,
8 bits 8 bits 8 bits 8 bits

The commands are sent continuously during a rather long time period (e.g. 30 seconds or more). When a new increase or decrease step is desired the Count byte is increased by one to allow the implanted control unit 338 to decode and understand that another step is demanded by the external control unit 336. If any part of the digital packet is erroneous, its content is simply ignored.

Through a line 340, an implanted energizer unit 326 draws energy from the high frequency electromagnetic wave signal received by the receiving antenna 334. The energizer unit 326 stores the energy in a power supply, such as a large capacitor, powers the control unit 338 and powers the electric motor 22 via a line 342.

The control unit 338 comprises a demodulator and a microprocessor. The demodulator demodulates digital signals sent from the external control unit 336. The microprocessor of the control unit 338 receives the digital packet, decodes it and, provided that the power supply of the energizer unit 326 has sufficient energy stored, sends a signal via a signal line 344 to the motor 22 to either increase or decrease the size of the restriction opening of the restriction member depending on the received command code.

Alternatively, the energy stored in the power supply of the energizer unit may only be used for powering a switch, and the energy for powering the motor 22 may be obtained from another implanted power source of relatively high capacity, for example a battery. In this case the switch is adapted to connect the battery to the control unit 338 in an “on” mode when the switch is powered by the power supply and to keep the battery disconnected from the control unit in a “standby” mode when the switch is not powered. With reference to FIG. 708-35, the remote control system schematically described-above will now be described in accordance with a more detailed embodiment. The external control unit 336 comprises a microprocessor 346, a signal generator 348 and a power amplifier 350 connected thereto. The microprocessor 346 is adapted to switch, the signal generator 348 on/off and to modulate signals generated by the signal generator 348 with digital commands that are sent to implanted components of the device of the invention. The power amplifier 350 amplifies the signals and sends them to the external signal transmitting antenna 332. The antenna 332 is connected in parallel with a capacitor 352 to form a resonant circuit tuned to the frequency generated by the signal generator 348.

The implanted signal receiving antenna coil 334 forms together with a capacitor 354 a resonant circuit that is tuned to the same frequency as the transmitting antenna 332. The signal receiving antenna coil 334 induces a current from the received high frequency electromagnetic waves and a rectifying diode 360 rectifies the induced current, which charges a storage capacitor 358. A coil 356 connected between the antenna coil 334 and the diode 360 prevents the capacitor 358 and the diode 360 from loading the circuit of the signal receiving antenna 334 at higher frequencies. Thus, the coil 356 makes it possible to charge the capacitor 358 and to transmit digital information using amplitude modulation. A capacitor 362 and a resistor 364 connected in parallel and a diode 366 forms a detector used to detect amplitude modulated digital information. A filter circuit is formed by a resistor 368 connected in series with a resistor 370 connected in series with a capacitor 372 connected in series with the resistor 368 via ground, and a capacitor 374, one terminal of which is connected between the resistors 368, 370 and the other terminal of which is connected between the diode 366 and the circuit formed by the capacitor 362 and resistor 364. The filter circuit is used to filter out undesired low and high frequencies. The detected and filtered signals are fed to an implanted microprocessor 376 that decodes the digital information and controls the motor 22 via an H-bridge 378 comprising transistors 380, 382, 384 and 386. The motor 22 can be driven in two opposite directions by the H-bridge 378. The microprocessor 376 also monitors the amount of stored energy in the storage capacitor 358. Before sending signals to activate the motor 22, the microprocessor 376 checks whether the energy stored in the storage capacitor 358 is enough. If the stored energy is not enough to perform the requested operation, the microprocessor 376 waits for the received signals to charge the storage capacitor 358 before activating the motor 22.

FIGS. 708-36A and 3708-6B show an embodiment of the apparatus of the aspect comprising a restriction device 402 having an elongated flexible restriction member 404, such as a belt, a cord or the like. The flexible member 404 extends in a loop around the tissue, suitably the urethra of the patient. (Alternatively, the flexible member 404 may comprise two separate parts on opposite sides of the urethra. One portion 404A of member 404 is attached to a frame 408 and another portion 04B of member 404 opposite portion 404A in the loop of the flexible member 404 is connected to an adjustment device 410, which is fixed to the frame 408. The adjustment device 410 pulls the flexible member 404 in the direction from portion 404A to squeeze the urethra between two opposite lengths of the flexible member 404 to thereby restrict the urine passageway in the urethra 406, see FIG. 708-36A, and releases the urethra from the flexible member 404 to thereby increase the urine passageway to allow the patient to urinate 406, see FIG. 708-36B.

FIGS. 708-37A and 708-37B show an embodiment of the apparatus comprising a restriction device 412 having two plate or bar elements 414 on opposite sides of the urethra 406. An adjustment device 416 moves the elements 412 in parallel towards each other to squeeze the urethra 406 between the elements 412 to thereby restrict the urine passageway in the urethra 406, see FIG. 708-37A, and moves the elements 412 away from each other to release the urethra 406, see FIG. 708-37B. FIG. 708-38 shows an embodiment of the apparatus comprising a restriction device 418 having two rigid articulated clamping elements 420 positioned on opposite sides of the urethra 406. An adjustment device 422 turns the clamping elements 420 toward each other to clamp the urethra 406 between the clamping elements 420 to thereby restrict the urine passageway in the urethra 406, and turns the clamping elements 420 away from each other to release the urethra 406 from the clamping elements 420 to thereby decrease the restriction of the urine passageway to allow the patient to urinate.

FIGS. 708-39A, 708-39B and 708-39C show an embodiment of the apparatus comprising a restriction device 424 having three bending members in the form of cylindrical rollers 426, 428 and 430 displaced relative one another in a row along the urethra 406 and positioned alternately on opposite sides of the urethra 406. (Alternatively, each roller 426, 428 and 430 may take the shape of an hourglass.) An adjustment, device 432 moves the two outer rollers 426, 430 laterally, against the urethra 406 in one direction and the intermediate roller 428 against the urethra 406 in the opposite direction to bend the urethra to thereby restrict the urine passageway in the urethra 406, see FIG. 708-39B. To increase the urine passageway to allow the patient to urinate 406, the adjustment device 432 moves the rollers 426-430 away from the urethra 406 to release it from the rollers 426-430, see FIG. 708-39C.

FIGS. 708-40A through 708-44B schematically illustrates modifications of the above embodiment according to FIGS. 708-39A-708-39C. Thus, FIGS. 708-40A and 708-40B show an embodiment similar to that of FIGS. 708-39A-708-39C except that the bending members are oval and not rotatable. FIGS. 708-41A and 708-41B show an embodiment similar to that of FIGS. 708-40A and 708-40B except that the oval bending members are rotatable to release the urethra, see FIG. 708-41A, and squeeze the urethra, see FIG. 708-41B.

FIGS. 708-42A and 708-42B show an embodiment similar to that of FIGS. 708-39A-708-39C except that the intermediate roller has a changeable diameter to release the urethra, see FIG. 708-42A, and squeeze the urethra, see FIG. 708-42B.

FIGS. 708-43A and 708-43B show an embodiment similar to that of FIGS. 708-37A-708-37C except that the elements are replaced by two cylindrical rollers positioned on opposite sides of the urethra.

Finally, FIGS. 708-44A and 708-44B show an embodiment substantially similar to that of FIGS. 708-43A and 708-43B except that the restriction device is curved to form an S-shaped curvature of the urethra.

FIG. 708-45 schematically illustrates how any of the above-described embodiments of the urinary incontinence treatment apparatus may be implanted in a patient. Thus, the apparatus comprises an adjustable restriction device 434 extending around the urethra 435 of the patient and a motor operated adjustment device for mechanically adjusting the restriction device 434 to squeeze the urethra or urine bladder to thereby restrict the urine passageway. The motor, not shown, is integrated in the adjustment device and is reversible to operate the adjustment device to release the urethra or urine bladder from the restriction device 434 to allow the patient to urinate. A wireless remote control of the apparatus comprises an external signal transmitter 438 incorporated in a portable remote-control unit and an implanted signal receiver 440, which comprises a control unit for controlling the adjustment device in response to a control signal, for example an electromagnetic wave signal, from the transmitter 438. The signal receiver 440 further comprises an energizer unit which transforms energy from the control signal transmitted by the transmitter 438 into electric energy for energy consuming implanted components of the apparatus, such as the motor for operating the adjustment device. The electric energy is conducted via an implanted conductor 442 from the signal receiver 440 to the motor. When the patient needs to urinate, he readily uses the portable remote-control unit to activate the implanted adjustment device to temporarily adjust the implanted restriction device 434 to start releasing the urine passageway to allow him to urinate. A pressure sensor 439 is implanted for sensing the pressure on the restriction device 434. The control unit of the signal receiver 449 controls the adjustment device to release the restriction device 434 in response to the pressure sensor 439 sensing an abnormal high pressure. There are a number of other conceivable alternative embodiments that give the same result as the above-described embodiments. For example, the microprocessor of the external and implanted, respectively, control unit may be replaced by discrete components. The power amplifier of the external control unit may be omitted if the signals generated by the signal generator are strong enough. Therefore the invention is to be accorded the broadest interpretation of the appended claims to encompass all equivalent structures and assemblies.

### AG: Urinary Incontinence Treatment Apparatus 2

FIG. 709-1 schematically shows an embodiment of the urinary incontinence treatment apparatus having some parts implanted in a patient and other parts located outside the patient's body. Thus, in FIG. 709-1 all parts placed to the right of the patient's skin 2 are implanted and all parts placed to the left of the skin 2 are located outside the patient's body. The apparatus of FIG. 709-1 comprises an implanted operable restriction device 4, which engages the patient's urethra (or alternatively the urine bladder) to form a restricted urine passageway. The restriction device 4 is capable of performing a reversible function, i.e. to open and close the urine passageway. An implanted control unit 6 controls the restriction device 4 via a control line 8 to form an adequate restriction of the of the urine passageway. An external control unit 10 includes an external source of energy and a wireless remote control transmitting a control signal generated by the external source of energy. The control signal is received by a signal receiver incorporated in the implanted control unit 6, whereby the control unit 6 controls the implanted restriction device 4 in response to the control signal. The implanted control unit 6 also uses energy from the control signal for operating the restriction device 4 via a power supply line 12.

FIG. 709-2 shows an embodiment of the aspect identical to that of FIG. 709-1, except that a reversing device in the form of a switch 14 operable by energy also is implanted in the patient for reversing the restriction device 4. The control unit 6 uses the switch 14 to reverse the function performed by the restriction device 4. More precisely, the external control unit 10 releases energy carried by a wireless signal and the implanted control unit 6 transforms the wireless energy into a current for operating the switch 14. When the control unit 6 shifts the polarity of the current the switch 14 reverses the function performed by the restriction device 4.

FIG. 709-3 shows an embodiment of the aspect identical to that of FIG. 709-1, except that an operation device in the form of a motor 16 also is implanted in the patient. The implanted control unit 6 powers the motor 16 with wireless energy released from the external source of energy of the external control unit 10. The implanted control unit 6 controls the operation of the motor 16 in response to a control signal from the remote control of the external control unit 10.

FIG. 709-4 shows an embodiment of the aspect identical to that of FIG. 709-1, except that an assembly 16 including a motor/pump unit 18 and a fluid reservoir 20 also is implanted in the patient. In this case the restriction device 4 is hydraulically operated, i.e. hydraulic fluid is pumped by the motor/pump unit 18 from the reservoir 20 through a conduit 22 to the restriction device 4 to restrict the urine passageway, and hydraulic fluid is pumped by the motor/pump unit 18 back from the restriction device 4 to the reservoir 20 to enlarge the urine passageway. The external control unit 10 releases energy carried by a wireless signal and the implanted control unit 6 transforms the wireless energy into a current, for example a current, for powering the motor/pump unit 18 via an electric power supply line 2. The implanted control unit 6 controls the motor/pump unit 16 and the restriction device 4 via control lines 26 and 27.

FIG. 709-5 shows an embodiment of the aspect comprising the restriction device 4, hydraulically operated, and the implanted control unit 6, and further comprising a hydraulic fluid reservoir 230, a motor/pump unit 232 and a reversing device in the form of a hydraulic valve shifting device 234, all of which are implanted in the patient. The motor of the motor/pump unit 232 is an electric motor.

FIG. 709-6 shows an embodiment of the aspect identical to that of FIG. 709-1, except that an accumulator 28 also is implanted in the patient. The control unit 6 stores energy received from the external control unit 10 in the accumulator 28. In response to a control signal from the external control unit 10 the implanted control unit 6 releases energy from the accumulator 28 via a power line 30 for the operation of the restriction device 4.

FIG. 709-7 shows an embodiment comprising the restriction device 4, hydraulically operated, and the implanted control unit 6, and further comprising a source of energy in the form of a battery 32, a hydraulic fluid reservoir 34, a motor/pump unit 36 and a reversing device in the form of a hydraulic valve shifting device 38, all of which are implanted in the patient. The motor of the motor/pump unit 36 is an electric motor. An external control unit 40 includes a wireless remote control transmitting a control signal which is received by the signal receiver incorporated in the implanted control unit 6.

In response to a control signal from the external control unit 40 the implanted control unit 6 powers the motor/pump unit 36 with energy from the battery 32, whereby the motor/pump unit 36 distributes hydraulic fluid between the reservoir 34 and the restriction device 4. The control unit 6 controls the shifting device 38 to shift the hydraulic fluid flow direction between one direction in which the fluid is pumped by the motor/pump unit 36 from the reservoir 34 to the restriction device 4 to restrict the urine passageway, and another opposite direction in which the fluid is pumped by the motor/pump unit 36 back from the restriction device 4 to the reservoir 34 to enlarge the urine passageway. FIG. 709-8 shows an embodiment identical to that of FIG. 709-6, except that a battery 42 is substituted for the accumulator 28, the external control unit 40 of the embodiment of FIG. 709-5 is substituted for the external control unit 10 and an electric motor 44 is implanted in the patient for operating the restriction device 4. In response to a control signal from the external control unit 40 the implanted control unit 6 powers the motor 44 with energy from the battery 42, whereby the motor 44 operates the restriction device 4.

FIG. 709-9 shows an embodiment identical to that of FIG. 709-8, except that the motor/pump unit 36 of the embodiment of FIG. 709-7 is substituted for the motor 44 and a fluid reservoir 46 also is implanted in the patient. The reservoir 46 is via fluid conduits 48 and 50 connected to the motor/pump unit 36 and restriction device 4, which in this case is hydraulically operated. In response to a control signal from the external control unit 40, the implanted control unit 6 powers the electric motor of the motor/pump unit 36 with energy from the battery 42, whereby the motor/pump unit 36 distributes hydraulic fluid between the fluid reservoir 46 and the restriction device 4.

FIG. 709-10 shows an embodiment identical to that of FIG. 709-8, except that a mechanical reversing device in the form of a gearbox 52 also is implanted in the patient. The implanted control unit 6 controls the gearbox 52 to reverse the function performed by the restriction device 4 (mechanically operated). FIG. 709-11 shows an embodiment comprising the restriction device 4, the external control unit 10, an implanted source of energy 236 and an implanted switch 238. The switch 238 is operated by wireless energy released from the external source of energy of the external control unit 6 to switch between an off mode, in which the implanted source of energy 236 is not in use, and an on mode, in which the implanted source of energy 236 supplies energy for the operation of the restriction device 4.

FIG. 709-12 shows an embodiment identical to that of FIG. 709-11, except that also the control unit 6 is implanted, in order to receive a control signal from the wireless remote control of the external control unit 10. The switch 238 is operated by the wireless energy from the external source of energy 10 to switch between an off mode, in which the implanted source of energy 236 and the wireless remote control of the external control unit 10 are not in use, i.e. the control unit 6 is not capable of receiving the control signal, and a standby mode, in which the wireless remote control is permitted to control the internal source of energy 236, via the implanted control unit 6, to supply energy for the operation of the restriction device 4. FIG. 709-13 shows an embodiment identical to that of FIG. 709-12, except that an energy transforming device for transforming the wireless energy into storable energy is incorporated in the implanted control unit 6 and that the implanted source of energy 236 is of a type that is capable of storing the storable energy. In this case, in response to a control signal from the external control unit 10, the implanted control unit 6 controls the switch 238 to switch from an off mode, in which the implanted source of energy 236 is not in use, to an on mode, in which the source of energy 36 supplies energy for the operation of the restriction device 4.

FIG. 709-14 shows an embodiment identical to that of FIG. 709-13, except that an energy storage device 240 also is implanted in the patient for storing the storable energy transformed from the wireless energy by the transforming device of the control unit 6. In this case, the implanted control unit 6 controls the energy storage device 240 to operate the switch 238 to switch between an off mode, in which the implanted source of energy 236 is not in use, and an on mode, in which the implanted source of energy 236 supplies energy for the operation of the restriction device 4.

FIG. 709-15 shows an embodiment identical to that of FIG. 709-13, except that a motor 242 and a mechanical reversing device in the form of a gearbox 244 also are implanted in the patient. The implanted control unit 6 controls the gearbox 244 to reverse the function performed by the restriction device 4 (mechanically operated), i.e. enlarging and restricting the urine passageway.

FIG. 709-16 schematically shows conceivable combinations of implanted components of the apparatus for achieving various communication possibilities. Basically, there are the implanted restriction device 4, the implanted control unit 6 and the external control unit 10 including the external source of energy and the wireless remote control. As already described above the remote control transmits a control signal generated by the external source of energy, and the control signal is received by a signal receiver incorporated in the implanted control unit 6, whereby the control unit 6 controls the implanted restriction device 4 in response to the control signal. A sensor 54 may be implanted in the patient for sensing a physical parameter of the patient, such as the pressure in the stomach. The control unit 6, or alternatively the external control unit 10, may control the restriction device 4 in response to signals from the sensor 54. A transceiver may be combined with the sensor 54 for sending information on the sensed physical parameter to the external control unit 10. The wireless remote control of the external control unit 10 may comprise a signal transmitter or transceiver and the implanted control unit 6 may comprise a signal receiver or transceiver. Alternatively, the wireless remote control of the external control unit 10 may comprise a signal receiver or transceiver and the implanted control unit 6 may comprise a signal transmitter or transceiver. The above transceivers, transmitters and receivers may be used for sending information or data related to the restriction device from inside the patient's body to the outside thereof. The motor 44 may be implanted for operating the restriction device 4 and also the battery 32 may be implanted for powering the motor 44. The battery 32 may be equipped with a transceiver for sending information on the charge condition of the battery. Those skilled in the art will realize that the above various embodiments according to FIGS. 709-1-709-15 could be combined in many different ways. For example, the energy operated switch 14 could be incorporated in any of the embodiments of FIGS. 709-4, 709-6, 709-8-709-10. The hydraulic shifting device 38 could be incorporated in any of the embodiments of FIGS. 709-4 and 9. The gearbox 52 could be incorporated in any of the embodiments of FIGS. 709-1, 709-6 and 709-8.

FIG. 709-17 illustrates how any of the above-described embodiments of the apparatus may be implanted in a patient. Thus, an assembly of the apparatus implanted in the patient comprises a restriction device 56 engaging the urethra 58, an operation device 60 for operating the restriction device 56 and an internal control unit 62, which includes a signal receiver, for controlling the operation device 61. An external control unit 64 includes a signal transmitter for transmitting a wireless control signal to the signal receiver of the implanted control unit 62. The implanted control unit 62 is capable of transforming signal energy from the control signal into electric energy for powering the operation device 60 and for energizing energy consuming implanted components of the apparatus. FIG. 18 shows the basic parts of a wireless remote control of the apparatus including an electric motor 128 for operating a restriction device, for example of the type illustrated in FIG. 17. In this case, the remote control is based on the transmission of electromagnetic wave signals, often of high frequencies in the order of 100 kHz-1 gHz, through the skin 130 of the patient. In FIG. 18, all parts placed to the left of the skin 130 are located outside the patient's body and all parts placed to the right of the skin 130 are implanted. Any suitable remote control system may be used. An external signal transmitting antenna 132 is to be positioned close to a signal receiving antenna 134 implanted close to the skin 130. As an alternative, the receiving antenna 134 may be placed for example inside the abdomen of the patient. The receiving antenna 134 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 132 comprises a coil having about the same restriction as the coil of the receiving antenna 134 but wound with a thick wire that can handle the larger currents that is necessary. The coil of the transmitting antenna 132 is tuned to the same specific high frequency as the coil of the receiving antenna 134. An external control unit 136 comprises a microprocessor, a high frequency electromagnetic wave signal generator and a power amplifier. The microprocessor of the control unit 136 is adapted to switch the generator on/off and to modulate signals generated by the generator to send digital information via the power amplifier and the antennas 132, 134 to an implanted control unit 138. To avoid that accidental random high frequency fields trigger control commands, digital signal codes are used. A conventional keypad placed on the external control unit 136 is connected to the microprocessor thereof. The keypad is used to order the microprocessor to send digital signals to activate the restriction device to either restrict or enlarge the urine passageway. The microprocessor starts a command by applying a high frequency signal on the antenna 132. After a short time, when the signal has energized the implanted parts of the control system, commands are sent to restrict or enlarge the urine passageway in predefined steps. The commands are sent as digital packets in the form illustrated below.
Start pattern, Command, Count, Checksum,
8 bits 8 bits 8 bits 8 bits

The commands are sent continuously during a rather long time period (e.g., about 30 seconds or more). When a new restrict or enlarge step is desired the Count byte is increased by one to allow the implanted control unit 138 to decode and understand that another step is demanded by the external control unit 136. If any part of the digital packet is erroneous, its content is simply ignored.

Through a line 140, an implanted energizer unit 126 draws energy from the high frequency electromagnetic wave signals received by the receiving antenna 134. The energizer unit 126 stores the energy in a power supply, such as a large capacitor, powers the control unit 138 and powers the electric motor 128 via a line 142.

The control unit 138 comprises a demodulator and a microprocessor. The demodulator demodulates digital signals sent from the external control unit 136. The microprocessor of the control unit 138 receives the digital packet, decodes it and, provided that the power supply of the energizer unit 126 has sufficient energy stored, sends a signal via a signal line 144 to the motor 128 to either contract or enlarge the restriction device depending on the received command code.

Alternatively, the energy stored in the power supply of the energizer unit may only be used for powering a switch, and the energy for powering the motor 128 may be obtained from another implanted power source of relatively high capacity, for example a battery. In this case the switch is adapted to connect said battery to the control unit 138 in an on mode when said switch is powered by said power supply and to keep said battery disconnected from the control unit in a standby mode when said switch is unpowered.

With reference to FIG. 709-19, the remote control schematically described above will now be described in accordance with a more detailed embodiment. The external control unit 136 comprises a microprocessor 146, a signal generator 148 and a power amplifier 150 connected thereto. The microprocessor 146 is adapted to switch the signal generator 148 on/off and to modulate signals generated by the signal generator 148 with digital commands that are sent to implanted components of the apparatus. The power amplifier 150 amplifies the signals and sends them to the external signal transmitting antenna 132. The antenna 132 is connected in parallel with a capacitor 152 to form a resonant circuit tuned to the frequency generated by the signal generator 148. The implanted signal receiving antenna coil 134 forms together with a capacitor 154 a resonant circuit that is tuned to the same frequency as the transmitting antenna 132. The signal receiving antenna coil 134 induces a current from the received high frequency electromagnetic waves and a rectifying diode 160 rectifies the induced current, which charges a storage capacitor 158. A coil 156 connected between the antenna coil 134 and the diode 160 prevents the capacitor 158 and the diode 160 from loading the circuit of the signal receiving antenna 134 at higher frequencies. Thus, the coil 156 makes it possible to charge the capacitor 158 and to transmit digital information using amplitude modulation. A capacitor 162 and a resistor 164 connected in parallel and a diode 166 forms a detector used to detect amplitude modulated digital information. A filter circuit is formed by a resistor 168 connected in series with a resistor 170 connected in series with a capacitor 172 connected in series with the resistor 168 via ground, and a capacitor 174, one terminal of which is connected between the resistors 168, 170 and the other terminal of which is connected between the diode 166 and the circuit formed by the capacitor 162 and resistor 164. The filter circuit is used to filter out undesired low and high frequencies. The detected and filtered signals are fed to an implanted microprocessor 176 that decodes the digital information and controls the motor 128 via an H-bridge 178 comprising transistors 180, 182, 184 and 186. The motor 128 can be driven in two opposite directions by the H-bridge 178. The microprocessor 176 also monitors the amount of stored energy in the storage capacitor 158. Before sending signals to activate the motor 128, the microprocessor 176 checks whether the energy stored in the storage capacitor 158 is enough. If the stored energy is not enough to perform the requested operation, the microprocessor 176 waits for the received signals to charge the storage capacitor 158 before activating the motor 128.

### AH: An Apparatus for Temporary Male Constriction

FIG. 710-1A is a schematic view of an apparatus for male contraception as illustrated in FIG. 710-1B. The apparatus 100 of FIG. 710-1B shows restriction of vas deference 200A, 200B (vasa deferentia) downstream the ampulla of vas deference 220A, 220B. The apparatus thereby is operable to temporarily prevent from reaching urethra and provide time-limited sterility. The apparatus 100 has a restriction device 120 adapted to constrict vas deference mechanically or hydraulically and a control device 150 for controlling the operation of the restriction device as it is operated with a schematically illustrated operation device 170. The control device 150 is subcutaneously located and includes an external part and an internal part. An energizer unit (energy transmission device) 180 is capable to supply the device with wireless transmitted energy to an energy transforming device 151 connected to an energy source 152 for supplying energy consuming parts of the apparatus with energy. An external remote control unit 190 is capable of communicating with the control device 150 internal control unit 153 of the control device. The external part 150A of the control device 150 includes functions needed for external operation such as an injection port for supply of hydraulic fluid when the constriction is hydraulically operated and an activation/deactivation button for operating the restriction device. The internal part of the control device 150B can include a number of functions needed to control and operate the restriction device 120. In a hydraulically operated restriction device 120 the control device 150 can include a pump 154 operable on a reservoir for hydraulic fluid (not shown), whereby transportation of fluid from the reservoir activates the restriction device to restrict vas deference and transportation back to the reservoir deactivates the restriction device to release vas deference.

FIG. 710-1C shows the apparatus of FIG. 710-1B without any control device. The restriction device 120 is of the same type as in FIG. 710-1B, but it is here adapted to restrict both vas deferens and the outlet ducts of the seminal vesicles. FIG. 710-1D shows an apparatus of FIG. 710-1B or 710-1C with a modified restriction device 120A operating with a stimulation device on both vas deference. The stimulation device is here represented by a set of electrodes. FIG. 710-1E shows an apparatus of FIG. 710-1B or 710-1C with a restriction device comprising a stimulation device 120A and a constriction device 120B controlled by the control device to restrict both vas deference by their combined actions. In One embodiment the construction device 120B is manually operated with a pump that operates on a reservoir to perform a construction on vas deference while the stimulation device operated by the control device stimulates vas deferens to obtaining the sperm transport blocking effect. FIG. 710-1F shows another variant of the apparatus of FIG. 710-1B, wherein the restriction device 120 includes two construction devices each adapted to constrict a vas deferens and an outlet duct of a seminal vesicle, respectively in order to both stop the flow of sperms and seminal fluid. The function of the control device and other parts are further explained together with the FIGS. 710-1G to 710-27C, below.

FIG. 710-1G illustrates a system for treating a disease comprising an apparatus 1010 of the present invention placed in the abdomen of a patient. An implanted energy-transforming device 302 is adapted to supply energy consuming components of the apparatus with energy via a power supply line 303. An external energy-transmission device 304 for non-invasively energizing the apparatus 10 transmits energy by at least one wireless energy signal. The implanted energy-transforming device 1002 transforms energy from the wireless energy signal into electric energy which is supplied via the power supply line 1003.

The wireless energy signal may include a wave signal selected from the following: a sound wave signal, an ultrasound wave signal, an electromagnetic wave signal, an infrared light signal, a visible light signal, an ultra violet light signal, a laser light signal, a micro wave signal, a radio wave signal, an x-ray radiation signal and a gamma radiation signal. Alternatively, the wireless energy signal may include an electric or magnetic field, or a combined electric and magnetic field. The wireless energy-transmission device 1004 may transmit a carrier signal for carrying the wireless energy signal. Such a carrier signal may include digital, analogue or a combination of digital and analogue signals. In this case, the wireless energy signal includes an analogue or a digital signal, or a combination of an analogue and digital signal. Generally speaking, the energy-transforming device 1002 is provided for transforming wireless energy of a first form transmitted by the energy-transmission device 1004 into energy of a second form, which typically is different from the energy of the first form. The implanted apparatus 10 is operable in response to the energy of the second form. The energy-transforming device 1002 may directly power the apparatus with the second form energy, as the energy-transforming device 1002 transforms the first form energy transmitted by the energy-transmission device 1004 into the second form energy. The system may further include an implantable accumulator, wherein the second form energy is used at least partly to charge the accumulator. Alternatively, the wireless energy transmitted by the energy-transmission device 1004 may be used to directly power the apparatus, as the wireless energy is being transmitted by the energy-transmission device 1004. Where the system comprises an operation device for operating the apparatus, as will be described below, the wireless energy transmitted by the energy-transmission device 1004 may be used to directly power the operation device to create kinetic energy for the operation of the apparatus. The wireless energy of the first form may comprise sound waves and the energy-transforming device 1002 may include a piezo-electric element for transforming the sound waves into electric energy. The energy of the second form may comprise electric energy in the form of a direct current or pulsating direct current, or a combination of a direct current and pulsating direct current, or an alternating current or a combination of a direct and alternating current. Normally, the apparatus comprises electric components that are energized with electrical energy. Other implantable electric components of the system may be at least one voltage level guard or at least one constant current guard connected with the electric components of the apparatus. Optionally, one of the energy of the first form and the energy of the second form may comprise magnetic energy, kinetic energy, sound energy, chemical energy, radiant energy, electromagnetic energy, photo energy, nuclear energy or thermal energy. Preferably, one of the energy of the first form and the energy of the second form is non-magnetic, non-kinetic, non-chemical, non-sonic, non-nuclear or non-thermal. The energy-transmission device may be controlled from outside the patient's body to release electromagnetic wireless energy, and the released electromagnetic wireless energy is used for operating the apparatus. Alternatively, the energy-transmission device is controlled from outside the patient's body to release non-magnetic wireless energy, and the released nonmagnetic wireless energy is used for operating the apparatus.

The external energy-transmission device 1004 also includes a wireless remote control having an external signal transmitter for transmitting a wireless control signal for non-invasively controlling the apparatus. The control signal is received by an implanted signal receiver which may be incorporated in the implanted energy-transforming device 1002 or be separate there from. The wireless control signal may include a frequency, amplitude, or phase modulated signal or a combination thereof. Alternatively, the wireless control signal includes an analogue or a digital signal, or a combination of an analogue and digital signal. Alternatively, the wireless control signal comprises an electric or magnetic field, or a combined electric and magnetic field. The wireless remote control may transmit a carrier signal for carrying the wireless control signal. Such a carrier signal may include digital, analogue or a combination of digital and analogue signals. Where the control signal includes an analogue or a digital signal, or a combination of an analogue and digital signal, the wireless remote control preferably transmits an electromagnetic carrier wave signal for carrying the digital or analogue control signals.

FIG. 710-2 illustrates the system of FIG. 710-1G in the form of a more generalized block diagram showing the apparatus 10, the energy-transforming device 1002 powering the apparatus 10 via power supply line 1003, and the external energy-transmission device 1004, The patient's skin 1005, generally shown by a vertical line, separates the interior of the patient to the right of the line from the exterior to the left of the line.

FIG. 710-3 shows an embodiment identical to that of FIG. 2, except that a reversing device in the form of an electric switch 1008 operable for example by polarized energy also is implanted in the patient for reversing the apparatus 10. When the switch is operated by polarized energy the wireless remote control of the external energy-transmission device 1004 transmits a wireless signal that carries polarized energy and the implanted energy-transforming device 1002 transforms the wireless polarized energy into a polarized current for operating the electric switch 1008. When the polarity of the current is shifted by the implanted energy-transforming device 1002 the electric switch 1008 reverses the function performed by the apparatus 10.

FIG. 710-4 shows an embodiment identical to that of FIG. 710-2, except that an operation device 1007 implanted in the patient for operating the apparatus 10 is provided between the implanted energy-transforming device 1002 and the apparatus 10. This operation device can be in the form of a motor 1007, such as an electric servomotor. The motor 1007 is powered with energy from the implanted energy-transforming device 1002, as the remote control of the external energy-transmission device 1004 transmits a wireless signal to the receiver of the implanted energy-transforming device 1002.

FIG. 710-5 shows an embodiment identical to that of FIG. 710-2, except that it also comprises an operation device is in the form of an assembly 1008 including a motor/pump unit 1009 and a fluid reservoir 1010 is implanted in the patient. In this case the apparatus 10 is hydraulically operated, i.e. hydraulic fluid is pumped by the motor/pump unit 1009 from the fluid reservoir 101 through a conduit 1011 to the apparatus 10 to operate the apparatus, and hydraulic fluid is pumped by the motor/pump unit 1009 back from the apparatus 10 to the fluid reservoir 1010 to return the apparatus to a starting position. The implanted energy-transforming device 1002 transforms wireless energy into a current, for example a polarized current, for powering the motor/pump unit 1009 via an electric power supply line 1012.

Instead of a hydraulically operated apparatus 10, it is also envisaged that the operation device comprises a pneumatic operation device. In this case, the hydraulic fluid can be pressurized air to be used for regulation and the fluid reservoir is replaced by an air chamber.

In all of these embodiments the energy-transforming device 1002 may include a rechargeable accumulator like a battery or a capacitor to be charged by the wireless energy and supplies energy for any energy consuming part of the system.

As an alternative, the wireless remote control described above may be replaced by manual control of any implanted part to make contact with by the patient's hand most likely indirect, for example a press button placed under the skin.

FIG. 710-6 shows an embodiment comprising the external energy-transmission device 1004 with its wireless remote control, the apparatus 10, in this case hydraulically operated, and the implanted energy-transforming device 1002, and further comprising a hydraulic fluid reservoir 1013, a motor/pump unit 1009 and a reversing device in the form of a hydraulic valve shifting device 1014, all implanted in the patient. Of course the hydraulic operation could easily be performed by just changing the pumping direction and the hydraulic valve may therefore be omitted. The remote control may be a device separated from the external energy-transmission device or included in the same. The motor of the motor/pump unit 1009 is an electric motor. In response to a control signal from the wireless remote control of the external energy-transmission device 1004, the implanted energy-transforming device 1002 powers the motor/pump unit 1009 with energy from the energy carried by the control signal, whereby the motor/pump unit 1009 distributes hydraulic fluid between the hydraulic fluid reservoir 1013 and the apparatus 10. The remote control of the external energy-transmission device 1004 controls the hydraulic valve shifting device 1014 to shift the hydraulic fluid flow direction between one direction in which the fluid is pumped by the motor/pump unit 1009 from the hydraulic fluid reservoir 313 to the apparatus 10 to operate the apparatus, and another opposite direction in which the fluid is pumped by the motor/pump unit 1009 back from the apparatus 10 to the hydraulic fluid reservoir 1013 to return the apparatus to a starting position.

FIG. 710-7 shows an embodiment comprising the external energy-transmission device 1004 with its wireless remote control, the apparatus 10, the implanted energy-transforming device 1002, an implanted internal control unit 1015 controlled by the wireless remote control of the external energy-transmission device 1004, an implanted accumulator 1018 and an implanted capacitor 1017. The internal control unit 1015 arranges storage of electric energy received from the implanted energy-transforming device 1002 in the accumulator 1018, which supplies energy to the apparatus 10. In response to a control signal from the wireless remote control of the external energy-transmission device 1004, the internal control unit 1015 either releases electric energy from the accumulator 1018 and transfers the released energy via power lines 1018 and 1019, or directly transfers electric energy from the implanted energy-transforming device 1002 via a power line 1020, the capacitor 1017, which stabilizes the electric current, a power line 1021 and the power line 1019, for the operation of the apparatus 10.

The internal control unit is preferably programmable from outside the patient's body. In a preferred embodiment, the internal control unit is programmed to regulate the apparatus 10 according to a pre-programmed time-schedule or to input from any sensor sensing any possible physical parameter of the patient or any functional parameter of the system.

In accordance with an alternative, the capacitor 1017 in the embodiment of FIG. 710-7 may be omitted. In accordance with another alternative, the accumulator 1018 in this embodiment may be omitted.

FIG. 710-8 shows an embodiment identical to that of FIG. 710-2, except that a battery 1022 for supplying energy for the operation of the apparatus 10 and an electric switch 1023 for switching the operation of the apparatus 10 also are implanted in the patient. The electric switch 1023 may be controlled by the remote control and may also be operated by the energy supplied by the implanted energy-transforming device 1002 to switch from an off mode, in which the battery 1022 is not in use, to an on mode, in which the battery 1022 supplies energy for the operation of the apparatus 10.

FIG. 710-9 shows an embodiment identical to that of FIG. 710-8, except that an internal control unit 1015 controllable by the wireless remote control of the external energy-transmission device 1004 also is implanted in the patient. In this case, the electric switch 1023 is operated by the energy supplied by the implanted energy-transforming device 1002 to switch from an off mode, in which the wireless remote control is prevented from controlling the internal control unit 1015 and the battery is not in use, to a standby mode, in which the remote control is permitted to control the internal control unit 1015 to release electric energy from the battery 1022 for the operation of the apparatus 10.

FIG. 710-10 shows an embodiment identical to that of FIG. 710-9, except that an accumulator 1018 is substituted for the battery 1022 and the implanted components are interconnected differently. In this case, the accumulator 1018 stores energy from the implanted energy-transforming device 1002. In response to a control signal from the wireless remote control of the external energy-transmission device 1004, the internal control unit 1015 controls the electric switch 1023 to switch from an off mode, in which the accumulator 1018 is not in use, to an on mode, in which the accumulator 1018 supplies energy for the operation of the apparatus 10. The accumulator may be combined with or replaced by a capacitor.

FIG. 710-11 shows an embodiment identical to that of FIG. 710-10, except that a battery 1022 also is implanted in the patient and the implanted components are interconnected differently. In response to a control signal from the wireless remote control of the external energy-transmission device 1004, the internal control unit 1015 controls the accumulator 1018 to deliver energy for operating the electric switch 1023 to switch from an off mode, in which the battery 1022 is not in use, to an on mode, in which the battery 1022 supplies electric energy for the operation of the apparatus 10. Alternatively, the electric switch 1023 may be operated by energy supplied by the accumulator 1018 to switch from an off mode, in which the wireless remote control is prevented from controlling the battery 1022 to supply electric energy and is not in use, to a standby mode, in which the wireless remote control is permitted to control the battery 1022 to supply electric energy for the operation of the apparatus 10. It should be understood that the switch 1023 and all other switches in this application should be interpreted in its broadest embodiment. This means a transistor, MCU, MCPU, ASIC, FPGA or a DA converter or any other electronic component or circuit that may switch the power on and off Preferably the switch is controlled from outside the body, or alternatively by an implanted internal control unit.

FIG. 710-12 shows an embodiment identical to that of FIG. 710-8, except that a motor 1007, a mechanical reversing device in the form of a gear box 1024, and an internal control unit 1015 for controlling the gear box 1024 also are implanted in the patient. The internal control unit 1015 controls the gear box 1024 to reverse the function performed by the apparatus 10 (mechanically operated). Even simpler is to switch the direction of the motor electronically. The gear box interpreted in its broadest embodiment may stand for a servo arrangement saving force for the operation device in favor of longer stroke to act.

FIG. 710-13 shows an embodiment identical to that of FIG. 710-19 except that the implanted components are interconnected differently. Thus, in this case the internal control unit 1015 is powered by the battery 1022 when the accumulator 1018, suitably a capacitor, activates the electric switch 1023 to switch to an on mode. When the electric switch 1023 is in its on mode the internal control unit 1015 is permitted to control the battery 1022 to supply, or not supply, energy for the operation of the apparatus 10.

FIG. 710-14 schematically shows conceivable combinations of implanted components of the apparatus for achieving various communication options. Basically, there are the apparatus 10, the internal control unit 1015, motor or pump unit 1009, and the external energy-transmission device 1004 including the external wireless remote control. As already described above the wireless remote control transmits a control signal which is received by the internal control unit 1015, which in turn controls the various implanted components of the apparatus. A feedback device, preferably comprising a sensor or measuring device 1025, may be implanted in the patient for sensing a physical parameter of the patient. The physical parameter may be at least one selected from the group consisting of pressure, volume, diameter, stretching, elongation, extension, movement, bending, elasticity, muscle contraction, nerve impulse, body temperature, blood pressure, blood flow, heartbeats and breathing. The sensor may sense any of the above physical parameters. For example, the sensor may be a pressure or motility sensor. Alternatively, the sensor 1025 may be arranged to sense a functional parameter. The functional parameter may be correlated to the transfer of energy for charging an implanted energy source and may further include at least one selected from the group of parameters consisting of; electricity, any electrical parameter, pressure, volume, diameter, stretch, elongation, extension, movement, bending, elasticity, temperature and flow. The feedback may be sent to the internal control unit or out to an external control unit preferably via the internal control unit. Feedback may be sent out from the body via the energy transfer system or a separate communication system with receiver and transmitters. The internal control unit 1015, or alternatively the external wireless remote control of the external energy-transmission device 1004, may control the apparatus 10 in response to signals from the sensor 1025. A transceiver may be combined with the sensor 1025 for sending information on the sensed physical parameter to the external wireless remote control. The wireless remote control may comprise a signal transmitter or transceiver and the internal control unit 1015 may comprise a signal receiver or transceiver. Alternatively, the wireless remote control may comprise a signal receiver or transceiver and the internal control unit 1015 may comprise a signal transmitter or transceiver. The above transceivers, transmitters and receivers may be used for sending information or data related to the apparatus 10 from inside the patient's body to the outside thereof. Where the motor/pump unit 1009 and battery 1022 for powering the motor/pump unit 1009 are implanted, information related to the charging of the battery 1022 may be fed back. To be more precise, when charging a battery or accumulator with energy feedback information related to said charging process is sent and the energy supply is changed accordingly.

FIG. 710-15 shows an alternative embodiment wherein the apparatus 10 is regulated from outside the patient's body. The system 1000 comprises a battery 1022 connected to the apparatus 10 via a subcutaneous electric switch 1026. Thus, the regulation of the apparatus 10 is performed non-invasively by manually pressing the subcutaneous switch, whereby the operation of the apparatus 10 is switched on and off. It will be appreciated that the shown embodiment is a simplification and that additional components, such as an internal control unit or any other part disclosed in the present application can be added to the system. Two subcutaneous switches may also be used. In the preferred embodiment one implanted switch sends information to the internal control unit to perform a certain predetermined performance and when the patient press the switch again the performance is reversed.

FIG. 710-16 shows an alternative embodiment, wherein the system 1000 comprises a hydraulic fluid reservoir 1013 hydraulically connected to the apparatus. Non-invasive regulation is performed by manually pressing the hydraulic reservoir connected to the apparatus. The system may include an external data communicator and an implantable internal data communicator communicating with the external data communicator. The internal communicator feeds data related to the apparatus or the patient to the external data communicator and/or the external data communicator feeds data to the internal data communicator.

FIG. 710-17 schematically illustrates an arrangement of the system that is capable of sending information from inside the patient's body to the outside thereof to give feedback information related to at least one functional parameter of the apparatus or system, or related to a physical parameter of the patient, in order to supply an accurate amount of energy to an implanted internal energy receiver 1002 connected to implanted energy consuming components of the apparatus 10. Such an energy receiver 1002 may include an energy source and/or an energy-transforming device. Briefly described, wireless energy is transmitted from an external energy source 1004a located outside the patient and is received by the internal energy receiver 1002 located inside the patient. The internal energy receiver is adapted to directly or indirectly supply received energy to the energy consuming components of the apparatus 10 via a switch 1026. An energy balance is determined between the energy received by the internal energy receiver 1002 and the energy used for the apparatus 10, and the transmission of wireless energy is then controlled based on the determined energy balance. The energy balance thus provides an accurate indication of the correct amount of energy needed, which is sufficient to operate the apparatus 10 properly, but without causing undue temperature rise. In FIG. 710-17 the patient's skin is indicated by a vertical line 1005. Here, the energy receiver comprises an energy-transforming device 1002 located inside the patient, preferably just beneath the patient's skin 1005. Generally speaking, the implanted energy-transforming device 1002 may be placed in the abdomen, thorax, muscle fascia (e.g. in the abdominal wall), subcutaneously, or at any other suitable location. The implanted energy-transforming device 1002 is adapted to receive wireless energy E transmitted from the external energy-source 1004a provided in an external energy-transmission device 1004 located outside the patient's skin 1005 in the vicinity of the implanted energy-transforming device 1002.

As is well known in the art, the wireless energy E may generally be transferred by means of any suitable Transcutaneous Energy Transfer (TET) device, such as a device including a primary coil arranged in the external energy source 1004a and an adjacent secondary coil arranged in the implanted energy-transforming device 1002. When an electric current is fed through the primary coil, energy in the form of a voltage is induced in the secondary coil which can be used to power the implanted energy consuming components of the apparatus, e.g. after storing the incoming energy in an implanted energy source, such as a rechargeable battery or a capacitor. However, the present invention is generally not limited to any particular energy transfer technique, TET devices or energy sources, and any kind of wireless energy may be used. The amount of energy received by the implanted energy receiver may be compared with the energy used by the implanted components of the apparatus. The term “energy used” is then understood to include also energy stored by implanted components of the apparatus. A control device includes an external control unit 1004b that controls the external energy source 1004a based on the determined energy balance to regulate the amount of transferred energy. In order to transfer the correct amount of energy, the energy balance and the required amount of energy is determined by means of a determination device including an implanted internal control unit 1015 connected between the switch 1026 and the apparatus 10. The internal control unit 1015 may thus be arranged to receive various measurements obtained by suitable sensors or the like, not shown, measuring certain characteristics of the apparatus 10, somehow reflecting the required amount of energy needed for proper operation of the apparatus 10. Moreover, the current condition of the patient may also be detected by means of suitable measuring devices or sensors, in order to provide parameters reflecting the patient's condition. Hence, such characteristics and/or parameters may be related to the current state of the apparatus 10, such as power consumption, operational mode and temperature, as well as the patient's condition reflected by parameters such as; body temperature, blood pressure, heartbeats and breathing. Other kinds of physical parameters of the patient and functional parameters of the device are described elsewhere. Furthermore, an energy source in the form of an accumulator 1018 may optionally be connected to the implanted energy-transforming device 1002 via the control unit 1015 for accumulating received energy for later use by the apparatus 10. Alternatively or additionally, characteristics of such an accumulator, also reflecting the required amount of energy, may be measured as well. The accumulator may be replaced by a rechargeable battery, and the measured characteristics may be related to the current state of the battery, any electrical parameter such as energy consumption voltage, temperature, etc. In order to provide sufficient voltage and current to the apparatus 10, and also to avoid excessive heating, it is clearly understood that the battery should be charged optimally by receiving a correct amount of energy from the implanted energy-transforming device 1002, i.e. not too little or too much. The accumulator may also be a capacitor with corresponding characteristics. For example, battery characteristics may be measured on a regular basis to determine the current state of the battery, which then may be stored as state information in a suitable storage means in the internal control unit 1015. Thus, whenever new measurements are made, the stored battery state information can be updated accordingly. In this way, the state of the battery can be “calibrated” by transferring a correct amount of energy, so as to maintain the battery in an optimal condition.

Thus, the internal control unit 1015 of the determination device is adapted to determine the energy balance and/or the currently required amount of energy, (either energy per time unit or accumulated energy) based on measurements made by the above-mentioned sensors or measuring devices of the apparatus 10, or the patient, or an implanted energy source if used, or any combination thereof. The internal control unit 1015 is further connected to an internal signal transmitter 1027, arranged to transmit a control signal reflecting the determined required amount of energy, to an external signal receiver 1004c connected to the external control unit 1004b. The amount of energy transmitted from the external energy source 1004a may then be regulated in response to the received control signal. Alternatively, the determination device may include the external control unit 1004b. In this alternative, sensor measurements can be transmitted directly to the external control unit 1004b wherein the energy balance and/or the currently required amount of energy can be determined by the external control unit 1004b, thus integrating the above-described function of the internal control unit 1015 in the external control unit 1004b. In that case, the internal control unit 1015 can be omitted and the sensor measurements are supplied directly to the internal signal transmitter 1027 which sends the measurements over to the external signal receiver 1004c and the external control unit 1004b. The energy balance and the currently required amount of energy can then be determined by the external control unit 1004b based on those sensor measurements. Hence, the present solution according to the arrangement of FIG. 710-17 employs the feedback of information indicating the required energy, which is more efficient than previous solutions because it is based on the actual use of energy that is compared to the received energy, e.g. with respect to the amount of energy, the energy difference, or the energy receiving rate as compared to the energy rate used by implanted energy consuming components of the apparatus. The apparatus may use the received energy either for consuming or for storing the energy in an implanted energy source or the like. The different parameters discussed above would thus be used if relevant and needed and then as a tool for determining the actual energy balance. However, such parameters may also be needed per se for any actions taken internally to specifically operate the apparatus. The internal signal transmitter 1027 and the external signal receiver 10004c may be implemented as separate units using suitable signal transfer means, such as radio, IR (Infrared) or ultrasonic signals. Alternatively, the internal signal transmitter 1027 and the external signal receiver 1004c may be integrated in the implanted energy-transforming device 1002 and the external energy source 1004a, respectively, so as to convey control signals in a reverse direction relative to the energy transfer, basically using the same transmission technique. The control signals may be modulated with respect to frequency, phase or amplitude. Thus, the feedback information may be transferred either by a separate communication system including receivers and transmitters or may be integrated in the energy system. In accordance with the present invention, such an integrated information feedback and energy system comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a power switch for switching the connection of the internal first coil to the first electronic circuit on and off, such that feedback information related to the charging of the first coil is received by the external energy transmitter in the form of an impedance variation in the load of the external second coil, when the power switch switches the connection of the internal first coil to the first electronic circuit on and off. In implementing this system in the arrangement of FIG. 710-17, the switch 1026 is either separate and controlled by the internal control unit 1015, or integrated in the internal control unit 1015. It should be understood that the switch 1026 should be interpreted in its broadest embodiment. This means a transistor, MCU, MCPU, ASIC FPGA or a DA converter or any other electronic component or circuit that may switch the power on and off. To conclude, the energy supply arrangement illustrated in FIG. 710-17 may operate basically in the following manner. The energy balance is first determined by the internal control unit 1015 of the determination device. A control signal reflecting the required amount of energy is also created by the internal control unit 1015, and the control signal is transmitted from the internal signal transmitter 1027 to the external signal receiver 1004c. Alternatively, the energy balance can be determined by the external control unit 1004b instead depending on the implementation, as mentioned above. In that case, the control signal may carry measurement results from various sensors. The amount of energy emitted from the external energy source 1004a can then be regulated by the external control unit 1004b, based on the determined energy balance, e.g. in response to the received control signal. This process may be repeated intermittently at certain intervals during ongoing energy transfer, or may be executed on a more or less continuous basis during the energy transfer. The amount of transferred energy can generally be regulated by adjusting various transmission parameters in the external energy source 1004a, such as voltage, current, amplitude, wave frequency and pulse characteristics. This system may also be used to obtain information about the coupling factors between the coils in a TET system even to calibrate the system both to find an optimal place for the external coil in relation to the internal coil and to optimize energy transfer. Simply comparing in this case the amount of energy transferred with the amount of energy received. For example if the external coil is moved the coupling factor may vary and correctly displayed movements could cause the external coil to find the optimal place for energy transfer. Preferably, the external coil is adapted to calibrate the amount of transferred energy to achieve the feedback information in the determination device, before the coupling factor is maximized. This coupling factor information may also be used as a feedback during energy transfer. In such a case, the energy system of the present invention comprises an implantable internal energy receiver for receiving wireless energy, the energy receiver having an internal first coil and a first electronic circuit connected to the first coil, and an external energy transmitter for transmitting wireless energy, the energy transmitter having an external second coil and a second electronic circuit connected to the second coil. The external second coil of the energy transmitter transmits wireless energy which is received by the first coil of the energy receiver. This system further comprises a feedback device for communicating out the amount of energy received in the first coil as a feedback information, and wherein the second electronic circuit includes a determination device for receiving the feedback information and for comparing the amount of transferred energy by the second coil with the feedback information related to the amount of energy received in the first coil to obtain the coupling factor between the first and second coils. The energy transmitter may regulate the transmitted energy in response to the obtained coupling factor. With reference to FIG. 710-18, although wireless transfer of energy for operating the apparatus has been described above to enable non-invasive operation, it will be appreciated that the apparatus can be operated with wire bound energy as well. Such an example is shown in FIG. 710-18, wherein an external switch 1026 is interconnected between the external energy source 1004a and an operation device, such as an electric motor 1007 operating the apparatus 10. An external control unit 1004b controls the operation of the external switch 1026 to effect proper operation of the apparatus 10.

FIG. 710-19 illustrates different embodiments for how received energy can be supplied to and used by the apparatus 10. Similar to the example of FIG. 710-17, an internal energy receiver 1002 receives wireless energy E from an external energy source 1004a which is controlled by a transmission control unit 1004b. The internal energy receiver 1002 may comprise a constant voltage circuit, indicated as a dashed box “constant V” in the figure, for supplying energy at constant voltage to the apparatus 10. The internal energy receiver 1002 may further comprise a constant current circuit, indicated as a dashed box “constant C” in the figure, for supplying energy at constant current to the apparatus 10. The apparatus 10 comprises an energy consuming part 10a, which may be a motor, pump, restriction device, or any other medical appliance that requires energy for its electrical operation. The apparatus 10 may further comprise an energy storage device 10b for storing energy supplied from the internal energy receiver 1002. Thus, the supplied energy may be directly consumed by the energy consuming part 10a, or stored by the energy storage device 10b, or the supplied energy may be partly consumed and partly stored. The apparatus 10 may further comprise an energy stabilizing unit 10c for stabilizing the energy supplied from the internal energy receiver 1002. Thus, the energy may be supplied in a fluctuating manner such that it may be necessary to stabilize the energy before consumed or stored. The energy supplied from the internal energy receiver 1002 may further be accumulated and/or stabilized by a separate energy stabilizing unit 1028 located outside the apparatus 10, before being consumed and/or stored by the apparatus 10. Alternatively, the energy stabilizing unit 1028 may be integrated in the internal energy receiver 1002. In either case, the energy stabilizing unit 1028 may comprise a constant voltage circuit and/or a constant current circuit. It should be noted that FIG. 710-17 and FIG. 710-19 illustrate some possible but non-limiting implementation options regarding how the various shown functional components and elements can be arranged and connected to each other. However, the skilled person will readily appreciate that many variations and modifications can be made within the scope of the present invention.

FIG. 710-20 schematically shows an energy balance measuring circuit of one of the proposed designs of the system for controlling transmission of wireless energy, or energy balance control system. The circuit has an output signal centered on 2.5V and proportionally related to the energy imbalance. The derivative of this signal shows if the value goes up and down and how fast such a change takes place. If the amount of received energy is lower than the energy used by implanted components of the apparatus, more energy is transferred and thus charged into the energy source. The output signal from the circuit is typically feed to an A/D converter and converted into a digital format. The digital information can then be sent to the external energy-transmission device allowing it to adjust the level of the transmitted energy. Another possibility is to have a completely analog system that uses comparators comparing the energy balance level with certain maximum and minimum thresholds sending information to external energy-transmission device if the balance drifts out of the max/min window. The schematic FIG. 710-20 shows a circuit implementation for a system that transfers energy to the implanted energy components of the apparatus of the present invention from outside of the patient's body using inductive energy transfer. An inductive energy transfer system typically uses an external transmitting coil and an internal receiving coil. The receiving coil, L1, is included in the schematic FIG. 710-3; the transmitting parts of the system are excluded. The implementation of the general concept of energy balance and the way the information is transmitted to the external energy transmitter can of course be implemented in numerous different ways. The schematic FIG. 710-20 and the above described method of evaluating and transmitting the information should only be regarded as examples of how to implement the control system.

### Circuit Details

In FIG. 710-20 the symbols Y1, Y2, Y3 and so on symbolize test points within the circuit. The components in the diagram and their respective values are values that work in this particular implementation which of course is only one of an infinite number of possible design solutions. Energy to power the circuit is received by the energy receiving coil L1. Energy to implanted components is transmitted in this particular case at a frequency of 25 kHz. The energy balance output signal is present at test point Y1.

Those skilled in the art will realize that the above various embodiments of the system could be combined in many different ways. For example, the electric switch 1008 of FIG. 710-3 could be incorporated in any of the embodiments of FIGS. 710-6-710-12, the hydraulic valve shifting device 1014 of FIG. 710-6 could be incorporated in the embodiment of FIG. 710-5, and the gear box 1024 could be incorporated in the embodiment of FIG. 710-4. Please observe that the switch simply could mean any electronic circuit or component.

The embodiments described in connection with FIGS. 710-17, 710-19 and 710-20 identify a method and a system for controlling transmission of wireless energy to implanted energy consuming components of an electrically operable apparatus. Such a method and system will be defined in general terms in the following.

A method is thus provided for controlling transmission of wireless energy supplied to implanted energy consuming components of an apparatus as described above. The wireless energy E is transmitted from an external energy source located outside the patient and is received by an internal energy receiver located inside the patient, the internal energy receiver being connected to the implanted energy consuming components of the apparatus for directly or indirectly supplying received energy thereto. An energy balance is determined between the energy received by the internal energy receiver and the energy used for the apparatus. The transmission of wireless energy E from the external energy source is then controlled based on the determined energy balance.

The wireless energy may be transmitted inductively from a primary coil in the external energy source to a secondary coil in the internal energy receiver. A change in the energy balance may be detected to control the transmission of wireless energy based on the detected energy balance change. A difference may also be detected between energy received by the internal energy receiver and energy used for the medical device, to control the transmission of wireless energy based on the detected energy difference.

When controlling the energy transmission, the amount of transmitted wireless energy may be decreased if the detected energy balance change implies that the energy balance is increasing, or vice versa. The decrease/increase of energy transmission may further correspond to a detected change rate.

The amount of transmitted wireless energy may further be decreased if the detected energy difference implies that the received energy is greater than the used energy, or vice versa. The decrease/increase of energy transmission may then correspond to the magnitude of the detected energy difference.

As mentioned above, the energy used for the medical device may be consumed to operate the medical device, and/or stored in at least one energy storage device of the medical device.

When electrical and/or physical parameters of the medical device and/or physical parameters of the patient are determined, the energy may be transmitted for consumption and storage according to a transmission rate per time unit which is determined based on said parameters. The total amount of transmitted energy may also be determined based on said parameters.

When a difference is detected between the total amount of energy received by the internal energy receiver and the total amount of consumed and/or stored energy, and the detected difference is related to the integral over time of at least one measured electrical parameter related to said energy balance, the integral may be determined for a monitored voltage and/or current related to the energy balance.

When the derivative is determined over time of a measured electrical parameter related to the amount of consumed and/or stored energy, the derivative may be determined for a monitored voltage and/or current related to the energy balance.

The transmission of wireless energy from the external energy source may be controlled by applying to the external energy source electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

In that case, the frequency of the electrical pulses may be substantially constant when varying the first and/or second time intervals. When applying electrical pulses, the electrical pulses may remain unchanged, except for varying the first and/or second time intervals. The amplitude of the electrical pulses may be substantially constant when varying the first and/or second time intervals. Further, the electrical pulses may be varied by only varying the lengths of first time intervals between successive leading and trailing edges of the electrical pulses.

A train of two or more electrical pulses may be supplied in a row, wherein when applying the train of pulses, the train having a first electrical pulse at the start of the pulse train and having a second electrical pulse at the end of the pulse train, two or more pulse trains may be supplied in a row, wherein the lengths of the second time intervals between successive trailing edge of the second electrical pulse in a first pulse train and leading edge of the first electrical pulse of a second pulse train are varied. When applying the electrical pulses, the electrical pulses may have a substantially constant current and a substantially constant voltage. The electrical pulses may also have a substantially constant current and a substantially constant voltage. Further, the electrical pulses may also have a substantially constant frequency. The electrical pulses within a pulse train may likewise have a substantially constant frequency.

The circuit formed by the first electric circuit and the external energy source may have a first characteristic time period or first time constant, and when effectively varying the transmitted energy, such frequency time period may be in the range of the first characteristic time period or time constant or shorter.

A system comprising an apparatus as described above is thus also provided for controlling transmission of wireless energy supplied to implanted energy consuming components of the apparatus. In its broadest sense, the system comprises a control device for controlling the transmission of wireless energy from an energy-transmission device, and an implantable internal energy receiver for receiving the transmitted wireless energy, the internal energy receiver being connected to implantable energy consuming components of the apparatus for directly or indirectly supplying received energy thereto. The system further comprises a determination device adapted to determine an energy balance between the energy received by the internal energy receiver and the energy used for the implantable energy consuming components of the apparatus, wherein the control device controls the transmission of wireless energy from the external energy-transmission device, based on the energy balance determined by the determination device.

Further, the system may comprise any of the following:
A primary coil in the external energy source adapted to transmit the wireless energy inductively to a secondary coil in the internal energy receiver.
The determination device is adapted to detect a change in the energy balance, and the control device controls the transmission of wireless energy based on the detected energy balance change
The determination device is adapted to detect a difference between energy received by the internal energy receiver and energy used for the implantable energy consuming components of the apparatus, and the control device controls the transmission of wireless energy based on the detected energy difference.
The control device controls the external energy-transmission device to decrease the amount of transmitted wireless energy if the detected energy balance change implies that the energy balance is increasing, or vice versa, wherein the decrease/increase of energy transmission corresponds to a detected change rate.
The control device controls the external energy-transmission device to decrease the amount of transmitted wireless energy if the detected energy difference implies that the received energy is greater than the used energy, or vice versa, wherein the decrease/increase of energy transmission corresponds to the magnitude of said detected energy difference.
The energy used for the apparatus is consumed to operate the apparatus, and/or stored in at least one energy storage device of the apparatus.
Where electrical and/or physical parameters of the apparatus and/or physical parameters of the patient are determined, the energy-transmission device transmits the energy for consumption and storage according to a transmission rate per time unit which is determined by the determination device based on said parameters. The determination device also determines the total amount of transmitted energy based on said parameters.
When a difference is detected between the total amount of energy received by the internal energy receiver and the total amount of consumed and/or stored energy, and the detected difference is related to the integral over time of at least one measured electrical parameter related to the energy balance, the determination device determines the integral for a monitored voltage and/or current related to the energy balance.
When the derivative is determined overtime of a measured electrical parameter related to the amount of consumed and/or stored energy, the determination device determines the derivative for a monitored voltage and/or current related to the energy balance.
The energy-transmission device comprises a coil placed externally to the human body, and an electric circuit is provided to power the external coil with electrical pulses to transmit the wireless energy. The electrical pulses have leading and trailing edges, and the electric circuit is adapted to vary first time intervals between successive leading and trailing edges and/or second time intervals between successive trailing and leading edges of the electrical pulses to vary the power of the transmitted wireless energy. As a result, the energy receiver receiving the transmitted wireless energy has a varied power.
The electric circuit is adapted to deliver the electrical pulses to remain unchanged except varying the first and/or second time intervals.
The electric circuit has a time constant and is adapted to vary the first and second time intervals only in the range of the first time constant, so that when the lengths of the first and/or second time intervals are varied, the transmitted power over the coil is varied.
The electric circuit is adapted to deliver the electrical pulses to be varied by only varying the lengths of first time intervals between successive leading and trailing edges of the electrical pulses.
The electric circuit is adapted to supplying a train of two or more electrical pulses in a row, said train having a first electrical pulse at the start of the pulse train and having a second electrical pulse at the end of the pulse train, and
the lengths of the second time intervals between successive trailing edge of the second electrical pulse in a first pulse train and leading edge of the first electrical pulse of a second pulse train are varied by the first electronic circuit.
The electric circuit is adapted to provide the electrical pulses as pulses having a substantially constant height and/or amplitude and/or intensity and/or voltage and/or current and/or frequency.
The electric circuit has a time constant, and is adapted to vary the first and second time intervals only in the range of the first time constant, so that when the lengths of the first and/or second time intervals are varied, the transmitted power over the first coil are varied.
The electric circuit is adapted to provide the electrical pulses varying the lengths of the first and/or the second time intervals only within a range that includes the first time constant or that is located relatively close to the first time constant, compared to the magnitude of the first time constant.

FIGS. 710-21-710-24 show in more detail block diagrams of four different ways of hydraulically or pneumatically powering an implanted apparatus according to the invention.

FIG. 710-21 shows a system as described above with. The system comprises an implanted apparatus 10 and further a separate regulation reservoir 1013, a one way pump 1009 and an alternate valve 1014.

FIG. 710-22 shows the apparatus 10 and a fluid reservoir 1013. By moving the wall of the regulation reservoir or changing the size of the same in any other different way, the adjustment of the apparatus may be performed without any valve, just free passage of fluid any time by moving the reservoir wall.

FIG. 710-23 shows the apparatus 10, a two way pump 1009 and the regulation reservoir 1013.

FIG. 710-24 shows a block diagram of a reversed servo system with a first closed system controlling a second closed system. The servo system comprises a regulation reservoir 1013 and a servo reservoir 1050. The servo reservoir 1050 mechanically controls an implanted apparatus 10 via a mechanical interconnection 1054. The apparatus has an expandable/contractable cavity. This cavity is preferably expanded or contracted by supplying hydraulic fluid from the larger adjustable reservoir 1052 in fluid connection with the apparatus 10. Alternatively, the cavity contains compressible gas, which can be compressed and expanded under the control of the servo reservoir 1050. The servo reservoir 1050 can also be part of the apparatus itself. In one embodiment, the regulation reservoir is placed subcutaneous under the patient's skin and is operated by pushing the outer surface thereof by means of a finger. This system is illustrated in FIGS. 710-25a-c. In FIG. 710-25a, a flexible subcutaneous regulation reservoir 1013 is shown connected to a bulge shaped servo reservoir 1050 by means of a conduit 1011. This bellow shaped servo reservoir 1050 is comprised in a flexible apparatus 10. In the state shown in FIG. 710-25a, the servo reservoir 1050 contains a minimum of fluid and most fluid is found in the regulation reservoir 1013. Due to the mechanical interconnection between the servo reservoir 1050 and the apparatus 10, the outer shape of the apparatus 10 is contracted, i.e., it occupies less than its maximum volume. This maximum volume is shown with dashed lines in the figure.

FIG. 710-25b shows a state wherein a user, such as the patient in with the apparatus is implanted, presses the regulation reservoir 1013 so that fluid contained therein is brought to flow through the conduit 1011 and into the servo reservoir 1050, which, thanks to its bellow shape, expands longitudinally. This expansion in turn expands the apparatus 10 so that it occupies its maximum volume, thereby constricting vas deference downstream the ampulla (not shown), which it contacts.

The regulation reservoir 1013 is preferably provided with means 1013a for keeping its shape after compression. This means, which is schematically shown in the figure, will thus keep the apparatus in a stretched position also when the user releases the regulation reservoir. In this way, the regulation reservoir essentially operates as an on/off switch for the system.

An alternative embodiment of hydraulic or pneumatic operation will now be described with reference to FIGS. 710-26 and 710-27a-c. The block diagram shown in FIG. 710-26 comprises with a first closed system controlling a second closed system. The first system comprises a regulation reservoir 1013 and a servo reservoir 1050. The servo reservoir 1050 mechanically controls a larger adjustable reservoir 1052 via a mechanical interconnection 1054. An implanted apparatus 10 having an expandable/contractable cavity is in turn controlled by the larger adjustable reservoir 1052 by supply of hydraulic fluid from the larger adjustable reservoir 1052 in fluid connection with the apparatus 10. An example of this embodiment will now be described with reference to FIG. 710-27a-c. Like in the previous embodiment, the regulation reservoir is placed subcutaneous under the patient's skin and is operated by pushing the outer surface thereof by means of a finger. The regulation reservoir 1013 is in fluid connection with a bellow shaped servo reservoir 1050 by means of a conduit 1011. In the first closed system 1013, 1011, 1050 shown in FIG. 710-31a, the servo reservoir 1050 contains a minimum of fluid and most fluid is found in the regulation reservoir 1013. The servo reservoir 1050 is mechanically connected to a larger adjustable reservoir 1052, in this example also having a bellow shape but with a larger diameter than the servo reservoir 1050. The larger adjustable reservoir 1052 is in fluid connection with the apparatus 10. This means that when a user pushes the regulation reservoir 1013, thereby displacing fluid from the regulation reservoir 1013 to the servo reservoir 1050, the expansion of the servo reservoir 1050 will displace a larger volume of fluid from the larger adjustable reservoir 1052 to the apparatus 10. In other words, in this reversed servo, a small volume in the regulation reservoir is compressed with a higher force and this creates a movement of a larger total area with less force per area unit.

Like in the previous embodiment described above with reference to FIGS. 710-25a-c, the regulation reservoir 1013 is preferably provided with means 1013a for keeping its shape after compression. This means, which is schematically shown in the figure, will thus keep the apparatus 10 in a stretched position also when the user releases the regulation reservoir. In this way, the regulation reservoir essentially operates as an on/off switch for the system.

### AI: Anal Incontinence Treatment Apparatus

FIGS. 711-1-711-3 show a preferred embodiment of the anal incontinence treatment apparatus comprising a restriction device having an elongated restriction member in the form of a circular resilient core 2 with two overlapping end portions 4, 6. The core 2 defines a substantially circular restriction opening and is enclosed in an elastic soft hose 8 except at a releasable and lockable joint 10 of the core 2, which when released enables application of the core 2 with its hose 8 around a tissue of a patient, such as the colon or rectum or anus. The materials of all of these elements are bio-compatible so that the patient' body will not reject them. A mechanical adjustment device 12 for mechanically adjusting the longitudinal extension of the core 2 to change the size of the restriction opening comprises a drive wheel 14 in frictional engagement with the overlapping end portions 4, 6 of the core 2. The drive wheel 14 is journalled on a holder 16 placed in the hose 8 and provided with two counter pressure rollers 18, 20 pressing the respective end portions 4, 6 of the core 2 against the drive wheel 14 to increase the frictional engagement there between. An electric motor 22 is connected to the drive wheel 14 via a long flexible drive shaft 24 and is moulded together with a remote controlled power supply unit 26 in a body 28 of silicone rubber. The length of the flexible drive shaft 34 is selected so that the body 28 can be placed in a desired position in the patient's body, suitably in the abdomen.

When the patient doesn't want to relieve himself (defecation), he controls the power supply unit 26 to power the electric motor 22 to turn the drive wheel 14 in one direction to reduce the diameter of the core 2, so that the colon or rectum is squeezed and the fecal passageway is restricted. When the patient wishes to relieve himself he controls the power supply unit 26 to power the electric motor 22 to turn the drive wheel 14 in the opposite direction to increase the diameter of the core 2, so that the fecal passageway is open.

Alternatively, a rack gear may be formed on one of the end portions 4, 6 of the core 2 and the drive wheel 14 may be replaced by a drive gear wheel connected to the other end portion of the core 2 and in mesh with the rack gear.

FIG. 711-4 shows an embodiment which is identical to the embodiment of FIGS. 711-1-711-3, except that the motor 22 is encapsulated in a lateral protrusion 30 of the hose 8 so that it is fixed to the core 2 and has a short drive shaft 32 onto which the drive wheel 14 is mounted, and that the motor 22 is positioned relative to the circular core 2 such that the drive shaft 32 extends radially thereto.

FIG. 711-5 shows an embodiment which likewise is identical to the embodiment of FIGS. 711-1-711-3, except that the motor 22 is encapsulated in the hose 8 so that it is fixed to the core 2 and has a short drive shaft 32, and that the motor 22 is positioned relative to the core 2 such that the drive shaft 32 extends substantially tangentially to the circular core 2. There is an angular gearing 34 connecting the drive shaft 32 to the drive wheel 14.

FIG. 711-6 shows a suitable arrangement for the motor 22 in the embodiment of FIG. 711-5, comprising a first clamping member 36 secured to one end portion of the core 2 and a second clamping member 38 secured to the other end portion 6 of the core 2. The motor 22 is secured to the first clamping member 36 and is operatively connected to a worm 40 via a gear transmission 42. The worm 40 is journalled at its opposite ends on holders 44 and 46, which are rigidly secured to the clamping member 36 and the motor 22, respectively. The second clamping member 38 has a pinion in mesh with the worm 40. When the motor 22 is powered the worm 40 rotates and will thereby pull the end portion 6 of the core 2 in one or the opposite longitudinal direction, so that the diameter of the substantially circular core 2 is either increased or decreased.

FIG. 711-7 shows an embodiment in which the elongated restriction member comprises a core 48 and a helical spring 50. A spring contracting means in the form of a flexible pulling member 52, i.e. a string, wire or cable, is connected to the core 48 at one end thereof and extends through the helical spring 50. A hydraulic motor in the form of a cylinder/piston unit 54 is adapted to pull the flexible pulling member 52 to contract the helical spring 50 against an arresting member 56, which is fixed relative to the core 48. A tube 58 hinged to the arresting member 56 extends between the cylinder/piston unit 54 and the arresting member 56, the flexible pulling member 52 running through the tube 58 and being connected to the piston of the cylinder/piston unit 54.

FIG. 711-8 shows a similar embodiment in which a hydraulic transmission conduit 59 is provided between two piston-cylinder assemblies 54, for use as the hydraulic motor/device in FIG. 711-7.

FIG. 711-9 shows an embodiment in which the restriction member comprises two elongated helical springs 60 and 62 having free ends, and a body 64 to which the springs 60, 62 are nonrotatably secured at their opposite ends. The body 64 comprises two separate parts secured to opposite end portions of the enclosing elastic hose 8 and is designed with a releasable and lockable joint between the separate parts. An adjustment device in the form of a drive shaft 66 has two opposite end portions connected to the helical springs 60, 62, respectively, at their free ends. The coils of the springs 60, 62 form left and right hand helices, respectively. A motor 68 is adapted to rotate the drive shaft 66 in one direction to enlarge the coils of the helical springs 60, 62 to longitudinally contract the springs 60, 62 and to rotate the drive shaft 66 in the opposite direction to reduce the size of the coils of the springs 60, 62 to longitudinally extend the springs 60, 62. Thus, the elongated helical springs 60, 62 defines a restriction opening, the size of which is increased when the springs 60, 62 are extended and decreased when the springs 60, 62 are contracted.

FIG. 711-10 shows an embodiment according to the invention which is identical to the embodiment of FIG. 711-9, except that the adjustment device comprises a gearing having an input shaft 72 and two opposite aligned output shafts 74 and 76 connected to the helical springs 60 and 62, respectively, at their free ends. The input shaft 72 is connected to the output shafts 74, 76 such that they rotate at opposite directions upon rotation of the input shaft 72. The coils of the springs 60, 62 form the same helices.

FIGS. 711-11-711-14 show an embodiment of the device in which a hydraulic motor comprises two interconnected cylinders 78 and 80 and two pistons 82 and 84 in the respective cylinders 78, 80. The cylinders 78, 80 have a common fluid supply inlet member 86, which together with the cylinders 78, 80 takes the shape of a Y-pipe. The restriction member comprises an elongated resilient arcuate core 88. The adjustment device comprises two bars 90 and 92 secured to opposite ends of the core 88 and connected to the pistons 82 and 84, respectively. The core 88 defines a restriction opening and is provided with a releasable and lockable joint 94 (FIG. 13) to permit application of the core 88 around the tissue. The core 88 and the cylinders 90, 92 are enclosed by a soft elastic hose 96 except at the joint 94 and the inlet member 86. The hose 96 has an outer tubular wall 98 and a central coaxial inner tubular wall 100, which is fixed to the outer wall 98 by spoke members 102 (FIG. 711-14). The core 88 is loosely fit in the inner tubular wall 100. By supplying fluid to or withdrawing fluid from the inlet 86 the pistons 82 and 84 will move towards or from each other, so that the restriction opening defined by the core 88 is changed by the longitudinal displacement of the bars 90, 92.

FIGS. 711-15-711-17 show an embodiment which is identical to the embodiment of FIGS. 711-11-711-14, except that the adjustment device comprises an elongated voltage responsive element 104 secured to the opposite ends of the core 88, so that the core 88 and the element 104 form the restriction member. The element 104 is capable of bending inwardly into a bow in response to a voltage applied across the element 104. The radius of curvature of the bow is adjustable by changing the level of the voltage applied to element 104.

FIG. 711-18 shows an embodiment comprising a loop forming means in the form of a substantially rigid outer circular element 106 with a releasable and lockable joint 108. In this embodiment the restriction member comprises an elastic inner circular element 110 formed by the innermost wall portion of an elastic hose 112 extending along the outer element 106. The inner circular element 110 is disposed concentrically within the outer circular element 106. The adjustment device comprises a plurality of threads 114 secured to the elastic inner element 110 along the circumference thereof and running from the inner element 110 via guide members 116 attached to the outer element 106. By pulling all the threads 114 the inner elastic element 110 is pulled under expansion radially outwardly towards the outer element 106.

FIG. 711-19 shows an embodiment which is identical to the embodiment of FIG. 711-9, except that it comprises a loop forming means in the form of a substantially rigid outer circular element 118 supporting the helical springs 60, 62, and a soft elastic inner wall 120 extending along the springs 60, 62. When the motor 68 rotates the helical springs 60, 62 in a direction that enlarges the coils of the springs 60, 62, the coils are forced by the rigid outer element 118 to expand radially inwardly thereby reducing the size of the restriction opening formed by the circumferential confinement surface of the restriction member (springs 60, 62 and body 64).

FIG. 711-20 shows an embodiment in which a restriction member comprises a plurality of arcuate lamellae 122 arranged like the conventional adjustable aperture mechanism of a camera. The adjustment device, not shown, is conventional and is operated by a motor 124 to adjust the lamellae 122 to change the size of an restriction opening defined by the lamellae 122.

FIGS. 711-21-711-23 show an embodiment in which a restriction member comprises two semi-circular elements 126 and 128 which are hinged together such that the semi-circular elements 126, 128 are swingable relative to each other between a fully open state in which they substantially form a circle, illustrated in FIG. 711-22 and an angular state, in which the size of the restriction opening defined by the semi-circular elements 126, 128 is reduced, illustrated in FIG. 711-23. The adjustment device, not shown, is conventional and is operated by a motor 130 to swing the semi-circular elements 126, 128 relative to each other.

FIGS. 711-24-711-27 show an embodiment in which a restriction member comprises an elastic belt 130 forming a circle and having a substantially oval cross-section. The restriction member 130 is provided with a releasable and lockable joint 132. An elastic double walled hose 134 encloses the belt 130 except at the joint 132. The adjustment device, not shown, is conventional and is operated by a motor 136 to turn the belt 130 around the longitudinal extension thereof between a fully open state, in which the inner broader side of the belt 130 forms a substantially cylindrical surface, illustrated in FIG. 711-26, and a reduced open state, in which the inner broader side of the belt 130 forms a substantially conical surface, illustrated in FIG. 711-27.

FIG. 711-28 schematically illustrates a cushion arrangement for protecting the human tissue, comprising a plurality of cushions 138 disposed in series along a substantially circular holding member 140. This cushion arrangement may be utilized in any of the above described embodiments of the invention.

FIGS. 711-29A-D provide a block diagram of four different hydraulic transmission configurations. FIG. 711-29A shows an adjustment device 202, a separate reservoir 204, a one way pump 206 and an alternate valve 208. FIG. 711-29B shows the adjustment device 202 and an adjustable reservoir 210. FIG. 711-29C shows the adjustment device 202, a two-way pump 212 and the reservoir 204. FIG. 711-29D shows a reverse servo system with a first closed system controlling a second system. The servo system comprises an adjustable servo reservoir 210 and a passive adjustable fluid supply reservoir 214. Any of the reservoirs can be the active reservoir, either the servo reservoir 210 or the fluid supply reservoir 214. The reservoir 214 controls a larger adjustable reservoir 216, which is used for the operation of the adjustment device 202 for changing the restriction opening of the restriction member.

FIGS. 711-30A-D are cross-sectional views of a pump mechanism adapted to pump fluid in both directions only by mechanically pushing a separate sealing wall portion 218 in one direction. FIG. 711-30A shows a piston 220 pushed forwards against a spring 222 towards the wall portion 218 and located in a pump housing 224 conducting fluid from a right upper fluid passage 226 of the housing 224 to a left fluid passage 228 of the housing 224. A main valve 230 is open and a nonreturn valve 232 is closed. FIG. 711-30B illustrates the first pump movement in which the piston 220 has moved forwards and reaches the wall portion 218. FIG. 711-30C illustrates how the piston 220 moves backwards by the action of the spring 222. The main valve 230 is now closed and the nonreturn valve 232 is open for fluid from the right upper passage 226. FIG. 711-30D illustrates how the piston 220 is moved further downwards from its position according to FIG. 711-30B while pushing the wall portion 218 downwards against a second spring 234 that is stronger than spring 222, so that fluid escapes from a right lower fluid passage 236. When moving the piston 220 backwards from the position of FIG. 711-30D, fluid enters the left fluid passage 228 and a valve 238 in the lower right fluid passage 236 closes.

FIG. 711-31 is a cross-sectional view of a reservoir 240 defining a chamber 242, the volume of which is variable and is controlled by a remote controlled motor 244, in accordance with FIG. 711-29B or 711-29D. The reservoir 240 and the motor 244 are placed in a housing 246. The chamber 242 is varied by moving a large wall 248. The wall 248 is secured to a nut 250, which is threaded on a rotatable spindle 252. The spindle 252 is rotated by the motor 244 via an angular gearing, which comprises two conical gear wheels 254 and 256 in mesh with each other. The motor 244 is powered by a battery 258 placed in the housing 246. A signal receiver 260 for controlling the motor 244 is also placed in the housing 246. Alternatively, the battery 258 and the signal receiver 260 may be mounted in a separate place. The signal receiver may comprise any known or conventional device which is capable of receiving a control signal and then operating the motor 244.

FIG. 711-32 is a cross-sectional view of a reservoir 262 defining a chamber 264, the volume of which is variable and is controlled by manual manipulation. A gable wall portion 266 of an open ended inner cylindrical housing 68 is adapted to be pushed downwards to fit in a desired locking groove 270 of a plurality of locking grooves 270 on the mantle wall of the cylindrical housing 268, to reduce the volume of the chamber 64. The inner cylindrical housing 268 is suspended by springs 272 and is telescopically applied on an outer cylindrical housing 274. When pushing the inner cylindrical housing 268 it moves downwards relative to the outer cylindrical housing 274 causing the gable wall portion 266 to release from the locking groove 270 and move upwards relative to the inner cylindrical housing 268. When the inner housing 268 is moved upwardly by the action of the springs 272 the volume of the chamber 264 is increased.

FIGS. 711-33A and 711-33B show a reverse servo comprising a main ring-shaped fluid reservoir 276 defining a chamber 278, the volume of which is variable. Centrally positioned in the main ring-shaped reservoir 276 there is a servo fluid reservoir 280 defining a chamber 282, the volume of which is variable. The chamber 282 of the servo reservoir 280 is significantly smaller than the chamber 278 of the main reservoir 276. The two reservoirs 276 and 280 are situated between two opposite separate walls 284 and 286, and are secured thereto. When changing the amount of fluid in the servo reservoir 280, the two opposite walls 284, 286 are moved towards or away from each other, whereby the volume of the chamber 278 of the main reservoir 276 is changed.

FIG. 711-34 shows the basic parts of a remote control system of the apparatus including a motor, for instance the electric motor 22. In this case, the remote control system is based on the transmission of an electromagnetic wave signal, often of a high frequency in the order of 100 kHz-1 gHz, through the skin 330 of the patient. In FIG. 34, all parts placed to the left of the skin 330 are located outside the patient's body and all parts placed to the right of the skin 330 are implanted in the patient's body.

An external signal transmitting antenna 332 is to be positioned close to a signal receiving antenna 334 implanted in the patient's body close to the skin 330. As an alternative, the receiving antenna 334 may be placed for example inside the abdomen of the patient. The receiving antenna 334 comprises a coil, approximately 1-100 mm, preferably 25 mm in diameter, wound with a very thin wire and tuned with a capacitor to a specific high frequency. A small coil is chosen if it is to be implanted under the skin of the patient and a large coil is chosen if it is to be implanted in the abdomen of the patient. The transmitting antenna 332 comprises a coil having about the same size as the coil of the receiving antenna 334 but wound with a thick wire that can handle the larger currents that is necessary. The coil of the transmitting antenna 332 is tuned to the same specific high frequency as the coil of the receiving antenna 334.

An external control unit 336 comprises a microprocessor, a high frequency electromagnetic signal generator and a power amplifier. The microprocessor of the control unit 336 is adapted to switch on/off the generator and to modulate signals generated by the generator to send digital information via the power amplifier and the antennas 332, 334 to an implanted control unit 338. To avoid that accidental random high frequency fields trigger control commands, digital signal codes are used. A keypad placed on the external control unit 336 is connected to the microprocessor thereof. The keypad is used to order the microprocessor to send a digital signal to either increase or decrease the size of the restriction opening defined by the loop of the restriction member (e.g. as described above). The microprocessor starts a command by applying a high frequency signal on the antenna 332. After a short time, when the signal has energized the implanted parts of the control system, commands are sent to increase or decrease the size of the restriction opening of the restriction member in predefined steps. The commands are sent as digital packets in the form illustrated below.

| | | | |
|---|---|---|---|
| Start pattern, | Command, | Count, | Checksum, |
| 8 bits | 8 bits | 8 bits | 8 bits |

The commands are sent continuously during a rather long time period (e.g. 30 seconds or more). When a new increase or decrease step is desired the Count byte is increased by one to allow the implanted control unit 338 to decode and understand that another step is demanded by the external control unit 336. If any part of the digital packet is erroneous, its content is simply ignored.

Through a line 340, an implanted energizer unit 326 draws energy from the high frequency electromagnetic wave signal received by the receiving antenna 334. The energizer unit 326 stores the energy in a power supply, such as a large capacitor, powers the control unit 338 and powers the electric motor 22 via a line 342.

The control unit 338 comprises a demodulator and a microprocessor. The demodulator demodulates digital signals sent from the external control unit 336. The microprocessor of the control unit 338 receives the digital packet, decodes it and, provided that the power supply of the energizer unit 326 has sufficient energy stored, sends a signal via a signal line 344 to the motor 22 to either increase or decrease the size of the restriction opening of the restriction member depending on the received command code.

Alternatively, the energy stored in the power supply of the energizer unit may only be used for powering a switch, and the energy for powering the motor 22 may be obtained from another implanted power source of relatively high capacity, for example a battery. In this case the switch is adapted to connect the battery to the control unit 338 in an “on” mode when the switch is powered by the power supply and to keep the battery disconnected from the control unit in a “standby” mode when the switch is unpowered.

With reference to FIG. 711-35, the remote control system schematically described above will now be described in accordance with a more detailed embodiment. The external control unit 336 comprises a microprocessor 346, a signal generator 348 and a power amplifier 350 connected thereto. The microprocessor 346 is adapted to switch the signal generator 348 on/off and to modulate signals generated by the signal generator 348 with digital commands that are sent to implanted components of the device of the invention. The power amplifier 350 amplifies the signals and sends them to the external signal transmitting antenna 332. The antenna 332 is connected in parallel with a capacitor 352 to form a resonant circuit tuned to the frequency generated by the signal generator 348.

The implanted signal receiving antenna coil 334 forms together with a capacitor 354 a resonant circuit that is tuned to the same frequency as the transmitting antenna 332. The signal receiving antenna coil 334 induces a current from the received high frequency electromagnetic waves and a rectifying diode 360 rectifies the induced current, which charges a storage capacitor 358. A coil 356 connected between the antenna coil 334 and the diode 360 prevents the capacitor 358 and the diode 360 from loading the circuit of the signal receiving antenna 334 at higher frequencies. Thus, the coil 356 makes it possible to charge the capacitor 358 and to transmit digital information using amplitude modulation.

A capacitor 362 and a resistor 364 connected in parallel and a diode 366 forms a detector used to detect amplitude modulated digital information. A filter circuit is formed by a resistor 368 connected in series with a resistor 370 connected in series with a capacitor 372 connected in series with the resistor 368 via ground, and a capacitor 374, one terminal of which is connected between the resistors 368, 370 and the other terminal of which is connected between the diode 366 and the circuit formed by the capacitor 362 and resistor 364. The filter circuit is used to filter out undesired low and high frequencies. The detected and filtered signals are fed to an implanted microprocessor 376 that decodes the digital information and controls the motor 22 via an H-bridge 378 comprising transistors 380, 382, 384 and 386. The motor 22 can be driven in two opposite directions by the H-bridge 378.

The microprocessor 376 also monitors the amount of stored energy in the storage capacitor 358. Before sending signals to activate the motor 22, the microprocessor 376 checks whether the energy stored in the storage capacitor 358 is enough. If the stored energy is not enough to perform the requested operation, the microprocessor 376 waits for the received signals to charge the storage capacitor 358 before activating the motor 22.

FIGS. 711-36A and 711-36B show an embodiment of the apparatus comprising a restriction device 402 having an elongated flexible restriction member 404, such as a belt, a cord or the like. The flexible member 404 extends in a loop around the tissue, suitably the rectum or colon or its prolongation. (Alternatively, the flexible member 404 may comprise two separate parts on opposite sides of the colon or rectum or the prolongation thereof). One portion 404A of member 404 is attached to a frame 408 and another portion 404B of member 404 opposite portion 404A in the loop of the flexible member 404 is connected to an adjustment device 410, which is fixed to the frame 408. The adjustment device 410 pulls the flexible member 404 in the direction from portion 404A to squeeze the colon or rectum or the prolongation thereof between two opposite lengths of the flexible member 404 to thereby restrict the fecal passageway in the colon or rectum 406 or the prolongation thereof, see FIG. 36A, and releases the colon or rectum or the prolongation thereof from the flexible member 404 to thereby increase the fecal passageway to allow defecation, see FIG. 711-36B.

FIGS. 711-37A and 711-37B show an embodiment of the apparatus comprising a restriction device 412 having two plate or bar elements 414 on opposite sides of the rectum 406. An adjustment device 416 moves the elements 412 in parallel towards each other to squeeze the rectum 406 between the elements 412 to thereby restrict the blood flow in the rectum 406, see FIG. 711-37A, and moves the elements 412 away from each other to release the rectum 406, see FIG. 711-37B.

FIG. 711-38 shows an embodiment of the apparatus comprising a restriction device 418 having two rigid articulated clamping elements 420 positioned on opposite sides of the rectum 406. An adjustment device 422 turns the clamping elements 420 toward each other to clamp the rectum 406 between the clamping elements 420 to thereby restrict the fecal passageway in the rectum 406, and turns the clamping elements 420 away from each other to release the rectum 406 from the clamping elements 420 to thereby increase the restriction of the fecal passageway to allow defecation.

FIGS. 711-39A, 711-39B and 711-39C show an embodiment of the apparatus comprising a restriction device 424 having three bending members in the form of cylindrical rollers 426, 428 and 430 displaced relative one another in a row along the rectum 406 and positioned alternately on opposite sides of the rectum 406. (Alternatively, each roller 426, 428 and 430 may take the shape of an hour-glass.) An adjustment device 432 moves the two outer rollers 426, 430 laterally against the rectum 406 in one direction and the intermediate roller 428 against the rectum 406 in the opposite direction to. bend the rectum to thereby restrict the fecal passageway in the rectum 406, see FIG. 39B. To increase the fecal passageway to allow defecation, the adjustment device 432 moves the rollers 426-430 away from the rectum 406 to release the latter from the rollers 426-430, see FIG. 39C.

FIGS. 711-40A through 711-44B schematically illustrates modifications of the above embodiment according to FIGS. 711-39A-711-39C. Thus, FIGS. 711-40A and 711-40B show an embodiment similar to that of FIGS. 39A-39C except that the bending members are oval and not rotatable. FIGS. 711-41A and 711-41B show an embodiment similar to that of FIGS. 711-40A and 711-40B except that the oval bending members are rotatable to release the rectum, see FIG. 711-41A, and squeeze the rectum, see FIG. 711-41B. FIGS. 711-42A and 711-42B show an embodiment similar to that of FIGS. 39A-39C except that the intermediate roller has a changeable diameter to release the rectum, see FIG. 711-42A, and squeeze the rectum, see FIG. 711-42B. FIGS. 711-43A and 711-43B show an embodiment similar to that of FIGS. 711-37A-711-37C except that the rigid elements are replaced by two cylindrical rollers positioned on opposite sides of the rectum. Finally, FIGS. 711-44A and 711-44B show an embodiment substantially similar to that of FIGS. 711-43A and 711-43B except that the restriction device is curved to form an S-shaped curvature of the rectum.

FIG. 711-45 schematically illustrates how any of the above-described embodiments of the anal incontinence treatment apparatus may be implanted in a patient. Thus, the apparatus comprises an adjustable restriction device 434 extending around the rectum 435 of the patient and a motor operated adjustment device 436 for mechanically adjusting the restriction device 434 to squeeze the rectum to thereby restrict the fecal passageway in the rectum. The motor, not shown, is integrated in the adjustment device 436 and—is reversible to operate the adjustment device 436 to release the rectum from the restriction device 434 to allow defecation. A wireless remote control of the apparatus comprises an external signal transmitter 438 incorporated in a portable remote-control unit and an implanted signal receiver 440, which comprises a control unit for controlling the adjustment device 436 in response to a control signal, for example an electromagnetic wave signal, from the transmitter 438. The signal receiver 440 further comprises an energizer unit which transfers energy from the control signal transmitted by the transmitter 438 into electric energy for energy consuming implanted components of the apparatus, such as the motor for operating the adjustment device 436. The electric energy is conducted via an implanted conductor 442 from the signal receiver 440 to the motor. When the patient needs to relieve himself (defecation), he readily uses the portable remote control unit to activate the implanted adjustment device 436 to temporarily adjust the implanted restriction device 434 to start release the fecal passageway to allow defecation. A pressure sensor 439 is implanted for sensing the pressure on the restriction device 434. The control unit of the signal receiver 449 controls the adjustment device 436 to release the restriction device 434 in response to the pressure sensor 439 sensing an abnormal high pressure. There are a number of other conceivable alternative embodiments that give the same result as the above-described embodiments. For example, the microprocessor of the external and implanted, respectively, control unit may be replaced by discrete components. The power amplifier of the external control unit may be omitted if the signals generated by the signal generator are strong enough. Therefore the invention is to be accorded the broadest interpretation of the appended claims to encompass all equivalent structures and assemblies.

### Legend

The following legend lists references used in some of the figures, description, and claims. Note that the same feature may have several labels or terms associated with it. The labels and terms in this legend are not to be seen as limiting and other nomenclature may be used in within this document in relation to specific references. The meanings of the labels and terms herein should chiefly be considered with reference to the definitions within this document and only secondarily with external uses and meanings. The references are primarily in the form of reference numerals. References for method steps are excluded from this legend.
C1-C3 Electrical/conductive/wired connection/communication 1-5
W1-W6 Wireless connection/communication 1-6
WS Wake signal
100 Implant
100a Internal control unit
101 Active unit
102 Communication unit
103 Wired transceiver/first transceiver/internal wired transceiver/first internal transceiver/internal transceiver/transceiver/wired transmitter
10a First power supply
10b Second power supply
104 Energy storage/Internal energy storage/internal energy source
104a First energy storage/first internal energy storage
104b Second energy storage/second internal energy storage
104c Energy source indicator
105 Energy receiver
105a First energy receiver
105b Second energy receiver
106 Computing unit/internal computing unit/processing unit/control unit
107 Memory/internal memory
108 Wireless transceiver/transceiver/internal wireless transceiver/transceiver/wireless receiver
1091 Wireless receiver/first wireless receiver
1092 Second wireless receiver
110 Control program/first control program
112 Second control program
114 Third control program
116 Reset function
118 Computer operating properly (COP) timer
118a First reset function
118b Second reset function
119 Energy provider
120 Energy source
120a Signal transmitter
121 Frequency detector
122 Receiver unit
1281 First communication system
1282 Second communication system
149 Feedback unit/internal feedback unit
150 Internal sensor/sensor/first sensor/at least one sensor
160 Internal clock
171 Implantable sensor/at least one sensor
172 Implantable manual receiver
173 Implantable switch
181 Sensation generator
181a Sensation generator to be worn in contact with the skin of a patient
181b Sensation generator generating the sensation without physical contact of a patient
182 Internal encryption unit/encryptor
183 Motor
200 External device
201 Conductive member/conduction member/electrical conduction element/communication unit
201a Case
203 Second wired transceiver/wired transceiver/external wired transceiver/first external transmitter/transceiver/communication unit
204 Energy storage/external energy storage
205 Energy transmitter/external charger/external energy source
206 Computing unit/external computing unit
207 Memory/external memory
208 Wireless transceiver/external wireless transceiver/wireless transmitter/transceiver/wireless receiver
2018 Wireless communication interface
2081 First wireless transceiver
2082 Second wireless transceiver
209 External encryption unit
210 External feedback unit
220 Verification unit
222 Fingerprint reader
250 External sensor/sensor/second sensor
260 External clock
270 Instruction provider
274 Remote control
280 Signal provider
281 Coil or magnet
282 Frequency indicator
290 External communication unit
300 Second external device/Controller
Energy storage unit
320′ Health Care Provider Patient External Interrogation Device
320″ Remote control
320′″ Patient External Interrogation Device
325 Energy transmitter
326 Computing unit
328 Wireless transceiver
330 Dedicated Data Infrastructure
332 Health Care Provider Dedicated Device
333 Hardware key
333′ Patient private key device
333″ Health Care Provider private key device
333′″ Master private key device
334 Display device
334i Control interface
335 Control buttons
336 Auxiliary device
339 NFC-transmitter
344 QR-code
395 Wireless receiver
411-422 Wireless connections
400 Third external device
500 Another external device (being the generator of the second key)
550 Sensor
57 Vascular portion
58 Needle operating device
59 Intestinal portion
60 Cardiac portion
61 Pulmonary portion
62 Urinary portion
800 At least one point (where destructive/constructive interference occurs)
801 First point/first transmitter
802 Second point/second transmitter
811 First slit
812 Second slit

### Numbered Embodiments

In the following, exemplifying numbered embodiments are provided and numbered, with Arabic numerals, in groups according to their aspect. The numbered embodiments are not to be seen as limiting the scope of the invention, which is defined by the appended claims. The reference numerals in the different numbered embodiments are to be seen only as examples of elements in the appended drawings which correspond to elements described in the numbered embodiments.

All embodiments or part of embodiments in the different aspects herein could be combined with any and/or all other embodiments or parts thereof in any order, thus comprising; embodiments or parts thereof in the same aspect in any order or combined with any and/or all embodiments or parts thereof in any different aspect in any order. The connection herein between the aspects and any of its embodiments or parts thereof are just examples and they are intended to be combined with each other in any combination or order. The embodiments or parts thereof may therefore be connected to each other in any order of connection between the embodiments or parts thereof.

Thus, all the different aspects or parts thereof could be combined with each other in any combination. Any and/or all embodiments or parts thereof in one aspect could be combined with any and/or all embodiments or parts thereof in any and/or all other aspects, embodiment(s) or parts thereof, described elsewhere in any order or combination.

### Aspect 309B eHealth Broadcasting Data

1. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implant comprising: at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
7. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
   the sensed parameter being above a predetermined threshold,
   the sensed parameter being below a predetermined threshold,
   the sensed parameter being outside of a predetermined range,
   a predetermined point in time,
   an expiry of a time period,
   a predetermined event, or
   a use of the implant, wherein
the implant comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
   the sensed parameter being above a predetermined threshold,
   the sensed parameter being below a predetermined threshold,
   the sensed parameter being outside of a predetermined range,
   a predetermined point in time,
   an expiry of a time period,
   a predetermined event, or
   a use of the implant, wherein
the implant comprises a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a system for treating a female patient to avoid or promote pregnancy comprising a restriction device adapted to postoperatively restrict and release an oviduct of the patient.
18. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The implant according to any one of embodiment 1-21 or 32-36, wherein the communication unit is configured to broadcast the information using a short to mid-range transmitting protocol.
23. The implant according to any one of embodiment 1-21 or 32-36, wherein the information is broadcasted using at least one of:
Radio Frequency type protocol
RFID type protocol
WLAN type protocol
Bluetooth type protocol
BLE type protocol
NFC type protocol
3G/4G/5G type protocol
GSM type protocol.
24. The implant according to any one of embodiment 1-21 or 32-36, wherein the implant further comprises a control unit connected to the sensor and to the communication unit, wherein the control unit is configured to anonymize the information.
25. The implant according to any one of embodiment 1-21 or 32-36, wherein the implant further comprises a control unit connected to the sensor and to the communication unit, wherein the control unit is configured to encrypt the information.
26. The implant according to any one of embodiment 1-21 or 32-36, wherein the communication unit further is configured to broadcast the information periodically.
27. The implant according to any one of embodiment 1-21 or 32-36, further comprising a control unit configured to cause the communication unit to broadcast the information in response to a second parameter being above a predetermined threshold.
28. The implant according to any one of embodiment 1-21 or 32-36, wherein the sensed parameter is at least one of a temperature, a pulse, a glucose level, an activity of an organ, or an acceleration.
29. The implant according to any one of embodiment 1-21 or 32-36, further comprising an implantable energy source and an energy source indicator, wherein the energy source indicator is configured to indicate a functional status of the implantable energy source.
30. The implant according to embodiment 29, wherein the functional status indicates at least one of charge level and temperature of the implantable energy source.
31. The implant according to any one of embodiment 1-21 or 32-36, wherein the functional parameter is a parameter relating to the internal control unit.
32. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
33. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
34. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
   the sensed parameter being above a predetermined threshold,
   the sensed parameter being below a predetermined threshold,
   the sensed parameter being outside of a predetermined range,
   a predetermined point in time,
   an expiry of a time period,
   a predetermined event, or
   a use of the implant, wherein
the implant comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
35. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
36. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises
a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 310C eHealth Double Encryption, Embodiments 1-30

1. A system comprising:
an implant comprising:
a communication unit configured to transmit data from the body of the patient to an external device, and
an encryption unit for encrypting the data to be transmitted, and
an external device configured to receive the data transmitted by the communication unit, authenticate, encrypt or authenticate and encrypt the received data using a first key and transmit the encrypted received data to a third device.
2. The system according to embodiment 1, wherein the encryption unit is configured to encrypt the data to be transmitted using a second key.
3. The system according to any of embodiments 1 or 2, wherein the first key or the second key is implant specific information, a secret key associated with the external device, an identifier of the implant or an identifier of the communication unit.
4. The system according to any of the preceding embodiments, wherein the second key is a key transmitted by the external device to the internal device.
5. The system according to any of embodiments 1-3, wherein the second key is a combined key comprising a third key received by the implant form the external device.
6. The system according to any preceding embodiment, wherein the first key is a combined key comprising a fourth key, wherein the fourth key is received by the external device from a verification unit connected to or comprised in the external device.
7. The system according to any preceding embodiment, wherein the verification unit is configured to receive authentication input from a user, for authenticating the communication between the implant and the external device.
8. The system according to embodiment 7, wherein the authentication input is a code.
9. The system according to embodiment 7, wherein the authentication input is based on a biometric technique selected from the list of: a fingerprint, a palm vein structure, image recognition, face recognition, iris recognition, a retinal scan, a hand geometry, and genome comparison.
10. The system according to embodiment 9, wherein the verification unit is configured to receive a fingerprint from a fingerprint reader.
11. A system according to any preceding embodiment, wherein the information is broadcasted using a short to mid-range transmitting protocol.
12. A system according to any preceding embodiment, wherein the information is transmitted using at least one of:
Radio Frequency type protocol
RFID type protocol
WLAN type protocol
Bluetooth type protocol
BLE type protocol
NFC type protocol
3G/4G/5G type protocol
GSM type protocol.
Bluetooth 5
13. A system according to any preceding embodiment, wherein the internal device comprises a first conductive member and the external device comprises a second conductive member, wherein the first and the second conductive members are configured to transmit the data using the body as a conductor.
14. A system according to any preceding embodiment, wherein the communication unit is configured to encrypt the data before transmitting the data.
15. A system according to embodiment 6 wherein the external device is configured to decrypt the received data and encrypt it before transmitting the data to the third device.
16. A system according to any preceding embodiment, wherein the external device is configured to transmit a request for data to the communication unit, and the communication unit is configured to in response to a request for data transmit the data to the external device.
17. A system according to any preceding embodiment, wherein the communication unit further is configured to broadcast the information periodically.
18. A system according to any preceding embodiment, further comprising an internal control unit configured to cause the communication unit to broadcast the information in response to a second parameter being above a predetermined threshold.
19. A method for encrypted communication between an implant, when implanted in a patient's body, and an external device, the method comprising:
   encrypting, by the implant, data relating to the implant or the operation thereof;
   transmitting, by a first communication unit comprised in the implant, the data;
   receiving, by a second communication unit comprised the external device, the data;
   encrypting, by the external device, the data using an encryption key to obtain encrypted data; and
   transmitting the encrypted data to a third external device.
20. The method according to embodiment 19, wherein the encrypting, by the implant, comprises encrypting the data using a second key.
21. The method according to embodiments 19 or 20, wherein the first or the second key is implant specific information, a secret key associated with the external device, an identifier of the implant or an identifier of the communication unit.
22. The method according to any of embodiments 19-21, wherein the second key is a key transmitted by the external device to the internal device.
23. The method according to any of embodiments 19-22, wherein the second key is a combined key comprising a third key; and the method further comprises: receiving, at the implant via a conductive member or wirelessly, the third key from the external device.
24. The method according to any of embodiments 19-23 further comprising: receiving, at the external device, a fourth key from a verification unit connected to or comprised in the external device,
wherein the verification unit is configured to receive authentication input from a user, for authenticating the communication between the implant and the external device, and
wherein the first key is a combined key comprising a fourth key.
25. The method according to ay of embodiments 19-24, wherein the authentication input is a code.
26. The method according to embodiment 25, wherein the authentication input is based on a biometric technique selected from the list of: a fingerprint, a palm vein structure, image recognition, face recognition, iris recognition, a retinal scan, a hand geometry, and genome comparison.
27. The method according to embodiment 25, wherein the verification unit is configured to receive a fingerprint from a fingerprint reader.
28. A computer program product comprising a computer-readable storage medium with instructions adapted to carry out the method of any one of embodiments 19-28 and/or with instructions adapted to carry out an action in any of the system embodiments 1-19, when executed by a computing unit in an external device having processing capability.
29. A computer program product comprising a computer-readable storage medium with instructions adapted to carry out the method of any one of embodiments 19-27 and/or with instructions adapted to carry out an action in any of the system embodiments 1-19, when executed by a computing unit in the implant having processing capability.
30. The implant according to any one of the following, alone or in any combination; system embodiments 1-19, with ability to perform method embodiments 19-28, and ability to use program product embodiments 28-29, comprising an internal control unit adapted to be involved in at least a part of the actions performed by the implant in at least a part of any one of the embodiments 1-29 above.
31. A system according to aspect 1-30 of Aspect 310SE comprising:
a second key placed in the implant adapted to authenticate, encrypt or authenticate and encrypt the data before being transmitted using the second key and transmit the encrypted received data to the external device.
32. A system according to aspect 31 of Aspect 310SE wherein at least one of the following:
a) the second key is a software key;
b) the second key is a hardware key;
c) the first key is a software key; and
d) the first key is a hardware key.
33. A system according to aspect 31-32 of Aspect 310SE, wherein the third device is adapted to authenticate the implant, the external device, or the implant and the external device, and decrypt the received data using a third key.
34. A system according to aspect 31-33 of Aspect 310SE, wherein the third key comprises at least one of the following:
a) a software key; and
b) a hardware key.
35. A system according to aspect 31-34 of Aspect 310SE, wherein the third device comprises: a storage unit adapted to store the received data.

### Aspect 310D eHealth Double Decryption Circuits, Embodiments 1-12

1. A system comprising:
an implant adapted to be implanted in a human or mammal comprising:
a communication unit configured to receive data from an external device, wherein the communication unit is controlled by a first chip, a first circuit, or a first chip and a first circuit,
a decryption unit for decrypting the data received, controlled by a second chip, a second circuit, or a second chip and a second circuit adapted to not be directly in contact with the external device,
a narrow secure communication tunnel between the first chip, a first circuit, or a first chip and a first circuit, and the second chip, a second circuit, or a second chip and a second circuit, wherein the communication unit is adapted to only communicate with the decryption unit via the narrow secure communication tunnel, and wherein the decryption unit is adapted to authenticate the original generator of the received data and authenticate that the data has not been tampered with.
2. A system according to aspect 1 of Aspect 310D, wherein the decryption unit is adapted to decrypt the received data and use the decrypted data after the decryption unit has authenticated the original generator of the received data and authenticated that the information has not been tampered with.
3. A system according to aspect 1-2 of Aspect 310D, wherein the implant comprises preprogrammed steps of action, wherein use of the decrypted data comprises instructions to select any one of the pre-programmed steps of actions of the implant.
4. A system according to aspect 1-3 of Aspect 310D, wherein use of the decrypted data comprises reprogramming part of a software of the implant to reprogram and add selectable preprogrammed steps of actions of the implant.
5. A system according to aspect 1-4 of Aspect 310D, wherein use of the decrypted data comprises reprogramming part of the implant software to change any one of usable information and actions of the implant.
6. A system according to aspect 1-5 of Aspect 310D, wherein the implant is adapted to receive data from the external device wherein the data is authenticated, encrypted or authenticated and encrypted before received.
7. A system according to aspect 1-6 of Aspect 310D, wherein a third device is adapted to authenticate, encrypt or authenticate and encrypt the data and adapted to transmit the information to the external device, wherein the external device is adapted to transmit the information to the implant untouched, relaying the information from the third device.
8. A system according to aspect 1-7 of Aspect 310D, wherein the implant is adapted to authenticate, decrypt or authenticate and decrypt the received data using a first key.
9. A system according to aspect 1-8 of Aspect 310D, wherein the external device is adapted to authenticate, encrypt, or authenticate and encrypt the transmitted data before transmitting the data, using a second key.
10. A system according to aspect 1-9 of Aspect 310D, wherein the third device is adapted to authenticate, encrypt, or authenticate and encrypt the transmitted data before transmitting the data, using a third key.
11. A system according to aspect 8-10 of Aspect 310D wherein at least one of the following:
a) the third key is a software key;
b) the third key is a hardware key;
c) the second key is a software key;
d) the second key is a hardware key;
e) the first key is a software key; and
f) the first key is a hardware key.
12. A system according to aspect 1-11 of Aspect 310D, wherein the decryption unit comprises a storage unit comprising information comprising at least one of authenticating information and key related information related to at least one of the external device and the third device, wherein the decryption unit is adapted to compare stored information with received data related to at least one of authentication and decryption.

### Aspect 311C eHealth Data Integrity

1. A method for evaluating a functional parameter of an implant implanted in a patient, the implant comprising a processor, a sensor for measuring the functional parameter, and an internal communication unit, the method comprising:
measuring, using the sensor, the functional parameter to obtain measurement data,
establishing a connection between the internal communication unit and an external device, a third device, an external device relying the data from a third device unchanged, or an external device and a third device configured to receive data from the implant,
determining, by the processor, a cryptographic hash or a metadata relating to the measurement data and adapted to be used by the external device to verify the integrity of the received data, and
transmitting the cryptographic hash or metadata, and
transmitting, from the communication unit, the measurement data.
2. The method according to embodiment 1, further comprising, at the external device,
receiving the transmitted cryptographic hash or metadata,
receiving the measurement data, and
verifying the integrity of the measurement data with the cryptographic hash, metadata or information relating to the functional parameter.
3. The method according to any of embodiments 1-2, wherein the cryptographic hash or metadata comprises a cryptographic hash, and wherein the verifying the integrity of the measurement data comprises:
   calculating a second cryptographic hash for the received measurement data using a same cryptographic hash algorithm as the processor, and
   determining that the measurement data has been correctly received based on that the cryptographic hash and the second cryptographic hash are equal.
4. The method according to embodiment 3, wherein the cryptographic hash algorithm comprises one of: MD5, SHA1, or SHA 256.
5. The method according to any of embodiments 3-4, wherein the cryptographic hash is a signature obtained by using a private key of the implant, and wherein the verifying, by the external device, comprises verifying the signature using a public key corresponding to the private key.
6. The method according to any of embodiments 2-5, wherein the cryptographic hash or metadata comprises a metadata, and wherein the verifying the integrity of the data comprises:
   obtaining a second metadata for the received measurement data relating to the functional parameter, and
   determining that the data has been correctly received based on that metadata and the second metadata are equal.
7. The method according to embodiment 5, wherein the metadata comprises: a length of the data, a timestamp, or a sensor measurement.
8. The method according to any preceding embodiment, further comprising, at the external device, evaluating the measurement data relating to the functional parameter.
9. The method according to any preceding embodiment, wherein the sensor is a pressure sensor, an electrical sensor, a clock, a temperature sensor, a motion sensor, an optical sensor, a sonic sensor, an ultrasonic sensor.
10. The method according to any preceding embodiment, wherein the functional parameter is at least one of a temperature, a pressure, a battery status indicator, a time period length, or a pressure at a sphincter.
11. The method according to any preceding embodiment, further comprising, at the external device, to determining, based on the evaluating, that the implant is functioning correctly.
12. The method according to any preceding embodiment, further comprising, at the external device, determining based on the evaluating that the implant is not functioning correctly.
13. The method according to embodiment 12, further comprising
   sending, from the external device, a corrective command to the implant,
   receiving the corrective command at the implant, and
   correcting the functioning of the implant according to the corrective command.
14. The method according to any embodiment,
wherein the transmitting of the measurement data is transmitted in a plurality of data packets,
wherein the cryptographic mash or metadata comprises a plurality of cryptographic hashes or metadata each corresponding to a respective data packet, and
wherein the transmitting of each the cryptographic hashes or metadata is performed for each of the corresponding data packets.
15. The method according to any preceding embodiment, wherein the method is for evaluating a pressure at a sphincter of the patient.
16. A method of communicating instructions from an external device to an implant implanted in a patient, the method comprising:
establishing a first connection between the external device and the implant,
establishing a second connection between a second external device and the implant,
transmitting, from the external device, a first set of instructions to the implant over the first connection,
transmitting, from the second external device, a first cryptographic hash or metadata corresponding to the first set of instructions to the implant,
at the implant, verifying the integrity of the first set of instructions and the first cryptographic hash, based on the first cryptographic hash.
17. The method according to embodiment 16, wherein the verifying of the integrity of the first set of instructions comprises a cyclic redundancy check.
18. The method according to any of embodiments 16-17, wherein the cryptographic hash or metadata comprises a cryptographic hash, and wherein the verifying the integrity of the first set of instructions comprises:
   calculating a second cryptographic hash for the received first set of instructions using a same cryptographic hash algorithm as the processor, and
   determining that the first set of instructions has been correctly received based on that the cryptographic hash and the second cryptographic hash are equal.
19. The method according to embodiment 18, wherein the cryptographic hash algorithm comprises at least one of MD5, SHA1, or SHA 256.
20. The method according to any of embodiments 16-17, wherein the cryptographic hash is a signature obtained by using a private key of the implant, and wherein the verifying comprises verifying the signature using a public key corresponding to the private key.
21. The method according to any of embodiment 17-20, wherein the cryptographic hash or metadata comprises a metadata, and wherein the verifying the integrity of the data comprises:
   obtaining a second metadata for the received first set of instructions, and
   determining that the first set of instructions has been correctly received based on that metadata and the second metadata are equal.
22. The method according to embodiment 21, wherein the metadata comprises at least one of a length of the data, and a timestamp.
32. The method according to any one of the preceding embodiments, wherein the external device is separate from the second external device.
33. The method according to any one of the preceding embodiments, wherein communication using the second connection is performed using a different protocol than a protocol used for communication using the first communication channel.
34. The method according to any one of the preceding embodiments, wherein the first connection is a wireless connection and the second connection is an electrical connection.
35. The method according to embodiment 34, wherein the second connection is an electrical connection using the patient's body as a conductor.
36. The method according to any preceding embodiment, further comprising:
transmitting, by the implant, information relating to the received first set of instructions,
receiving, by the external device, the information, and
verifying, by the external device, that the information corresponds to the first set of instructions sent by the external device.
37. The method according to embodiment 36, wherein the information comprises a length of the first set of instructions.
38. The method according to any preceding embodiment, further comprising:
at the implant, verifying the authenticity of the first set of instructions by
   i. calculating a second cryptographic hash for the first set of instructions,
   ii. comparing the second cryptographic hash with the first cryptographic hash,
   iii. determining that the first set of instructions are authentic based on that the second cryptographic hash is equal to the first cryptographic hash
upon verification of the authenticity of the first set of instructions, storing them at the implant.
39. The method according to any preceding embodiment, wherein the first set of instructions comprises a cryptographic hash corresponding to a previous set of instructions.
40. The method according to any preceding embodiment, further comprising:
measuring, by the implant using a first sensor, a parameter relating to the body of the patient to obtain a first measurement,
measuring, by the external device using a second sensor, the parameter relating to the body of the patient to obtain a second measurement,
wherein the first set of instructions comprises the second measurement relating to the body of the patient, and wherein the verification of the authenticity of the first set of instructions comprises comparing the first and the second measurements.
41. The method according to embodiment 40, wherein the first and second parameters relate to a pulse of the patient, a respiration rate of the patient, a temperature of the patient, a sound of the patient, or a physical movement of the patient.
42. The method according to embodiment 40 or 41, wherein the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.
43. A system for communication instructions, the system comprising:
   an implant adapted to be implanted in a patient, the implant comprising an active unit, an internal communication unit and an internal controller,
   an external device comprising an external communication unit configured to transmit a first set of instructions to the internal communication unit over a first communications connection,
   a second external device comprising a third communication unit configured to transmit a first cryptographic hash to the internal communication unit,
   wherein the internal controller is configured to receive, via the internal communication unit, the first set of instructions and the first cryptographic hash and verify the integrity of the first set of instructions based on the first cryptographic hash.
44. The system according to embodiment 43, wherein the internal controller is configured to verify the integrity of the first set of instructions using a cyclic redundancy check.
45. The system according to any of embodiments 43-44, wherein the cryptographic hash or metadata comprises a cryptographic hash, and wherein the internal controller is configured to verifying the integrity of the first set of instructions by:
   calculating a second cryptographic hash for the received first set of instructions using a same cryptographic hash algorithm as the processor, and
   determining that the first set of instructions has been correctly received based on that the cryptographic hash and the second cryptographic hash are equal.
46. The system according to embodiment 45, wherein the cryptographic hash algorithm comprises one of
47. The system according to any of embodiments 45-46, wherein the cryptographic hash is a signature obtained by using a private key of the implant, and wherein the internal controller is configured to verifying the first set of instructions by the signature using a public key corresponding to the private key.
48. The system according to any of embodiments 45-47, wherein the cryptographic hash or metadata comprises a metadata, and wherein the internal controller is configured to verifying the integrity of the data by:
   obtaining a second metadata for the received first set of instructions, and
   determining that the first set of instructions has been correctly received based on that metadata and the second metadata are equal.
49. The method according to embodiment 48, wherein the metadata comprises: a length of the data, a timestamp.
50. The system according to any of embodiments 43-49, wherein the external device is separate from the second external device.
51. The system according to any of embodiments 43-50, wherein the internal controller is configured to communicate with the second external device using a different protocol than a protocol used for communication with the external device.
52. The system according to any of embodiments 43-51, wherein the internal communication unit comprises a wireless transceiver for communication with the external device, and a conductive member for communicating with the second external device, wherein the second external device comprises a second conductive member.
53. The system according to embodiment 52, wherein the communication between the internal communication unit and the second external device is performed using the patient's body as a conductor.
54. The system according to any of embodiments 43-53, wherein the internal controller is configured to transmit information relating to the received first set of instructions to the external device, and the external device is configured to confirm that the information relates to the first set of instructions transmitted by the external device.
55. The system according to any of embodiments 43-54, wherein the internal controller is configured to:
   calculating a second cryptographic hash for the first set of instructions,
   comparing the second cryptographic hash with the first cryptographic hash,
   determining that the first set of instructions are authentic based on that the second cryptographic hash is equal to the first cryptographic hash, and
upon verification of the authenticity of the first set of instructions, storing them at the implant.
56. The system according to any of embodiments 43-55, wherein the external device is configured to transmit the first set of instructions, and wherein the first set of instructions comprises a cryptographic hash corresponding to a previous set of instructions.
57. The system according to any of embodiments 43-56, wherein
the internal controller is connected to or comprising a first sensor adapted to obtain a measurement of a parameter relating to the body of the patient,
the external device is connected to or comprising a second sensor adapted to obtain a measurement of the parameter relating to the body of the patient,
wherein the first set of instructions comprises the second measurement, and wherein the internal controller is configured to verify the authenticity of the first set of instructions at least based on a comparison of the first and second measurements.
58. The system according to embodiment 57, wherein the first and second parameters relate to a pulse of the patient, a respiration rate of the patient, a temperature of the patient, a sound of the patient, or a physical movement of the patient.
59. The system according to any of embodiments 57-58, wherein the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.
60. A computer program product comprising a computer-readable storage medium with instructions adapted to carry out the method of any one of embodiments 1-42 and/or with instructions adapted to carry out an action in any of the system embodiments 53-59, when executed by a computing unit in an external device having processing capability.
61. A computer program product comprising a computer-readable storage medium with instructions adapted to carry out the method of any one of embodiments 1-42 and/or with instructions adapted to carry out an action in any of the system embodiments 53-59, when executed by a computing unit in the implant having processing capability.
62. The implant according to any one of the following, alone or in any combination; system embodiments 43-59, with ability to perform method embodiments 1-42, and ability to use program product embodiments 60-61, comprising an internal control unit adapted to be involved in at least a part of the actions performed by the implant in at least a part of any one of the embodiments 1-61 above.

### Aspect 311D eHealth Data Integrity

1. A system for communicating with an implant when implanted in a mammal or patient, the implant comprising a processor, and an internal communication unit, the implant is adapted to communicate with a third device directly and indirectly being external to the body, wherein
the internal communication unit is adapted to receive data from the third device directly and indirectly, wherein the data is adapted to include a cryptographic hash or a metadata relating to the data adapted to be used by the internal communication unit to verify the authentication, unhampering of, or authentication and unhampering of the received data, wherein
the cryptographic hash or metadata is compared to the storage unit comprising such hash or data stored.
2. The method according to embodiment 1, further comprising, at the external device,
receiving the transmitted cryptographic hash or metadata,
receiving the measurement data, and
verifying the integrity of the measurement data with the cryptographic hash, metadata or information relating to the functional parameter.
3. The method according to any of embodiments 1-2, wherein the cryptographic hash or metadata comprises a cryptographic hash, and wherein the verifying the integrity of the measurement data comprises:
   calculating a second cryptographic hash for the received measurement data using a same cryptographic hash algorithm as the processor, and
   determining that the measurement data has been correctly received based on that the cryptographic hash and the second cryptographic hash are equal.
4. The method according to embodiment 3, wherein the cryptographic hash algorithm comprises one of MD5, SHA1, or SHA 256.
5. The method according to any of embodiments 3-4, wherein the cryptographic hash is a signature obtained by using a private key of the implant, and wherein the verifying, by the external device, comprises verifying the signature using a public key corresponding to the private key.
6. The method according to any of embodiments 2-5, wherein the cryptographic hash or metadata comprises a metadata, and wherein the verifying the integrity of the data comprises:
   obtaining a second metadata for the received measurement data relating to the functional parameter, and
   determining that the data has been correctly received based on that metadata and the second metadata are equal.
7. The method according to embodiment 5, wherein the metadata comprises: a length of the data, a timestamp, or a sensor measurement.
8. The method according to any preceding embodiment, further comprising, at the external device, evaluating the measurement data relating to the functional parameter.
9. The method according to any preceding embodiment, wherein the sensor is a pressure sensor, an electrical sensor, a clock, a temperature sensor, a motion sensor, an optical sensor, a sonic sensor, an ultrasonic sensor.
10. The method according to any preceding embodiment, wherein the functional parameter is at least one of a temperature, a pressure, a battery status indicator, a time period length, or a pressure at a sphincter.
11. The method according to any preceding embodiment, further comprising, at the external device, to determining, based on the evaluating, that the implant is functioning correctly.
12. The method according to any preceding embodiment, further comprising, at the external device, determining based on the evaluating that the implant is not functioning correctly.
13. The method according to embodiment 12, further comprising
   sending, from the external device, a corrective command to the implant,
   receiving the corrective command at the implant, and
   correcting the functioning of the implant according to the corrective command.
14. The method according to any embodiment,
wherein the transmitting of the measurement data is transmitted in a plurality of data packets,
wherein the cryptographic mash or metadata comprises a plurality of cryptographic hashes or metadata each corresponding to a respective data packet, and
wherein the transmitting of each the cryptographic hashes or metadata is performed for each of the corresponding data packets.
15. The method according to any preceding embodiment, wherein the method is for evaluating a pressure at a sphincter of the patient.
16. A method of communicating instructions from an external device to an implant implanted in a patient, the method comprising:
establishing a first connection between the external device and the implant,
establishing a second connection between a second external device and the implant,
transmitting, from the external device, a first set of instructions to the implant over the first connection,
transmitting, from the second external device, a first cryptographic hash or metadata corresponding to the first set of instructions to the implant,
at the implant, verifying the integrity of the first set of instructions and the first cryptographic hash, based on the first cryptographic hash.
17. The method according to embodiment 16, wherein the verifying of the integrity of the first set of instructions comprises a cyclic redundancy check.
18. The method according to any of embodiments 16-17, wherein the cryptographic hash or metadata comprises a cryptographic hash, and wherein the verifying the integrity of the first set of instructions comprises:
   calculating a second cryptographic hash for the received first set of instructions using a same cryptographic hash algorithm as the processor, and
   determining that the first set of instructions has been correctly received based on that the cryptographic hash and the second cryptographic hash are equal.
19. The method according to embodiment 18, wherein the cryptographic hash algorithm comprises at least one of MD5, SHA1, or SHA 256.
20. The method according to any of embodiments 16-17, wherein the cryptographic hash is a signature obtained by using a private key of the implant, and wherein the verifying comprises verifying the signature using a public key corresponding to the private key.
21. The method according to any of embodiment 17-20, wherein the cryptographic hash or metadata comprises a metadata, and wherein the verifying the integrity of the data comprises:
   obtaining a second metadata for the received first set of instructions, and
   determining that the first set of instructions has been correctly received based on that metadata and the second metadata are equal.
22. The method according to embodiment 21, wherein the metadata comprises at least one of: a length of the data, and a timestamp.
32. The method according to any one of the preceding embodiments, wherein the external device is separate from the second external device.
33. The method according to any one of the preceding embodiments, wherein communication using the second connection is performed using a different protocol than a protocol used for communication using the first communication channel.
34. The method according to any one of the preceding embodiments, wherein the first connection is a wireless connection and the second connection is an electrical connection.
35. The method according to embodiment 34, wherein the second connection is an electrical connection using the patient's body as a conductor.
36. The method according to any preceding embodiment, further comprising:
   transmitting, by the implant, information relating to the received first set of instructions,
   receiving, by the external device, the information, and
   verifying, by the external device, that the information corresponds to the first set of instructions sent by the external device.
37. The method according to embodiment 36, wherein the information comprises a length of the first set of instructions.
38. The method according to any preceding embodiment, further comprising: at the implant, verifying the authenticity of the first set of instructions by
   i. calculating a second cryptographic hash for the first set of instructions,
   ii. comparing the second cryptographic hash with the first cryptographic hash,
   iii. determining that the first set of instructions are authentic based on that the second cryptographic hash is equal to the first cryptographic hash upon verification of the authenticity of the first set of instructions, storing them at the implant.
39. The method according to any preceding embodiment, wherein the first set of instructions comprises a cryptographic hash corresponding to a previous set of instructions.
40. The method according to any preceding embodiment, further comprising:
measuring, by the implant using a first sensor, a parameter relating to the body of the patient to obtain a first measurement,
measuring, by the external device using a second sensor, the parameter relating to the body of the patient to obtain a second measurement,
wherein the first set of instructions comprises the second measurement relating to the body of the patient, and wherein the verification of the authenticity of the first set of instructions comprises comparing the first and the second measurements.
41. The method according to embodiment 40, wherein the first and second parameters relate to a pulse of the patient, a respiration rate of the patient, a temperature of the patient, a sound of the patient, or a physical movement of the patient.
42. The method according to embodiment 40 or 41, wherein the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.
43. A system for communication instructions, the system comprising:
   an implant adapted to be implanted in a patient, the implant comprising an active unit, an internal communication unit and an internal controller,
   an external device comprising an external communication unit configured to transmit a first set of instructions to the internal communication unit over a first communications connection,
   a second external device comprising a third communication unit configured to transmit a first cryptographic hash to the internal communication unit,
   wherein the internal controller is configured to receive, via the internal communication unit, the first set of instructions and the first cryptographic hash and verify the integrity of the first set of instructions based on the first cryptographic hash.
44. The system according to embodiment 43, wherein the internal controller is configured to verify the integrity of the first set of instructions using a cyclic redundancy check.
45. The system according to any of embodiments 43-44, wherein the cryptographic hash or metadata comprises a cryptographic hash, and wherein the internal controller is configured to verifying the integrity of the first set of instructions by:
   calculating a second cryptographic hash for the received first set of instructions using a same cryptographic hash algorithm as the processor, and
   determining that the first set of instructions has been correctly received based on that the cryptographic hash and the second cryptographic hash are equal.
46. The system according to embodiment 45, wherein the cryptographic hash algorithm comprises one of
47. The system according to any of embodiments 45-46, wherein the cryptographic hash is a signature obtained by using a private key of the implant, and wherein the internal controller is configured to verifying the first set of instructions by the signature using a public key corresponding to the private key.
48. The system according to any of embodiments 45-47, wherein the cryptographic hash or metadata comprises a metadata, and wherein the internal controller is configured to verifying the integrity of the data by:
   obtaining a second metadata for the received first set of instructions, and
   determining that the first set of instructions has been correctly received based on that metadata and the second metadata are equal.
49. The method according to embodiment 48, wherein the metadata comprises: a length of the data, a timestamp.
50. The system according to any of embodiments 43-49, wherein the external device is separate from the second external device.
51. The system according to any of embodiments 43-50, wherein the internal controller is configured to communicate with the second external device using a different protocol than a protocol used for communication with the external device.
52. The system according to any of embodiments 43-51, wherein the internal communication unit comprises a wireless transceiver for communication with the external device, and a conductive member for communicating with the second external device, wherein the second external device comprises a second conductive member.
53. The system according to embodiment 52, wherein the communication between the internal communication unit and the second external device is performed using the patient's body as a conductor.
54. The system according to any of embodiments 43-53, wherein the internal controller is configured to transmit information relating to the received first set of instructions to the external device, and the external device is configured to confirm that the information relates to the first set of instructions transmitted by the external device.
55. The system according to any of embodiments 43-54, wherein the internal controller is configured to:
   calculating a second cryptographic hash for the first set of instructions,
   comparing the second cryptographic hash with the first cryptographic hash,
   determining that the first set of instructions are authentic based on that the second cryptographic hash is equal to the first cryptographic hash, and
upon verification of the authenticity of the first set of instructions, storing them at the implant.
56. The system according to any of embodiments 43-55, wherein the external device is configured to transmit the first set of instructions, and wherein the first set of instructions comprises a cryptographic hash corresponding to a previous set of instructions.
57. The system according to any of embodiments 43-56, wherein
the internal controller is connected to or comprising a first sensor adapted to obtain a measurement of a parameter relating to the body of the patient,
the external device is connected to or comprising a second sensor adapted to obtain a measurement of the parameter relating to the body of the patient,
wherein the first set of instructions comprises the second measurement, and wherein the internal controller is configured to verify the authenticity of the first set of instructions at least based on a comparison of the first and second measurements.
58. The system according to embodiment 57, wherein the first and second parameters relate to a pulse of the patient, a respiration rate of the patient, a temperature of the patient, a sound of the patient, or a physical movement of the patient.
59. The system according to any of embodiments 57-58, wherein the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.
60. A computer program product comprising a computer-readable storage medium with instructions adapted to carry out the method of any one of embodiments 1-42 and/or with instructions adapted to carry out an action in any of the system embodiments 53-59, when executed by a computing unit in an external device having processing capability.
61. A computer program product comprising a computer-readable storage medium with instructions adapted to carry out the method of any one of embodiments 1-42 and/or with instructions adapted to carry out an action in any of the system embodiments 53-59, when executed by a computing unit in the implant having processing capability.
62. The implant according to any one of the following, alone or in any combination; system embodiments 43-59, with ability to perform method embodiments 1-42, and ability to use program product embodiments 60-61, comprising an internal control unit adapted to be involved in at least a part of the actions performed by the implant in at least a part of any one of the embodiments 1-61 above.

### Aspect 312C eHealth Programming Predefined Steps, Embodiments 1-46

1. An implant comprising:
an internal computing unit configured to control a function of said implant, said internal computing unit comprises an internal memory configured to store:
i. a first control program for controlling the internal computing unit, and
ii. a second, configurable or updatable or selectable, predefined program steps, control program for controlling said function of said implant,
iii. a set of predefined program steps for updating and selecting predefined program steps of the second control program,
an internal communication unit connected to said internal computing unit and configured to communicate with an external device, a third device, or an external device receiving a data from a third device, wherein said internal computing unit is configured to receive an update and selection of preprogrammed steps to the second control program via said internal communication unit, and
a verification unit or function of, connected to, or transmitted to said internal computing unit, said verification function being configured to verify that the received update and selection of preprogrammed steps to the second control program comprises program steps comprised in the set of predefined program steps.
2. The implant according to any preceding embodiment, wherein the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.
3. The implant according to any preceding embodiment, wherein the verification function is configured to reject the update in response to the update comprising program steps not comprised in the set of predefined program steps.
4. The implant according to any preceding embodiment, wherein the verification function is configured to allow the update in response to the update only comprising program steps comprised in the set of predefined program steps.
5. The implant according to embodiment 1, wherein the internal communication unit is configured to communicate with the external device via a first wireless connection for receiving the update to the second control program, and a second connection for performing an authentication of the communication with the external device.
6. The implant according to embodiment 5, wherein the second connection is a wireless short-range connection.
7. The implant according to embodiment 5 or 6, wherein the authentication second connection is an electrical connection using the patient's body as a conductor
8. The implant according to any preceding embodiment, wherein the internal computing unit is further configured to, upon verification, installing the update.
9. The implant according to any preceding embodiment, wherein the internal computing unit has a sleep mode and an active mode, and the implant further comprises a sensor configured to detect a wake signal, and wherein the implant is configured to in response to a detected wake signal set the internal computing unit to the active mode.
10. The implant according to embodiment 9, wherein sensor is configured to detect an acoustic signal as wake signal or wherein the sensor is configured to detect a magnetic signal as the wake signal
11. The implant according to any of embodiments 9-10, wherein
the sensor is configured to detect the received signal strength of a signal; and
the implant is further configured to set the internal computing unit to the active mode in response to the sensor detecting a signal exceeding a threshold signal strength.
12. The implant according to any of embodiments 9-11, further comprising a second internal computing unit, and wherein the implant is configured to set the internal computing unit to the active mode via the second internal computing unit.
13. The implant according to any of embodiments 9-12, wherein the internal computing unit in the sleep mode is substantially without power, and wherein setting the internal computing unit in the active mode comprises providing the internal computing unit with power.
14. The implant according to embodiment 13, wherein the implant comprises an energy controller for controlling the power supplied to the internal computing unit.
15. The implant according to embodiment 14, wherein the sensor is configured to provide the energy controller with a second wake signal in response to detecting the wake signal, and wherein the energy controller is configured to set the computing unit in the active mode in response to the second wake signal.
16. The implant according to any preceding embodiment, wherein
the sensor is configured to detect the received signal strength of a signal; and
the internal control unit is further configured to set the internal computing unit to the active mode in response to the sensor detecting a signal exceeding a threshold signal strength.
17. The implant according to any preceding embodiment, wherein
the wake signal comprises a predetermined signal pattern; and
the implant is further configured to set the processing unit to the active mode in response to the sensor detecting the predetermined signal pattern.
18. The implant according to any preceding embodiment, wherein the sensor is a hall effect sensor, a fluxgate sensor, an ultra-sensitive magnetic field sensor or a magneto-resistive sensor.
19. The implant according to any preceding embodiment, wherein the sensor comprises a third coil having an iron core.
20. The implant according to any preceding embodiment, wherein the sensor is comprised in the internal communication unit.
21. The implant according to any preceding embodiment, further comprising a frequency detector, communicatively coupled to the internal computing unit and configured to detect a frequency for data communication between the internal communication unit and an external device configured to transmit a frequency indicator signal.
22. The implant according to embodiment 21, wherein the frequency detector comprises an antenna.
23. The implant according to any preceding embodiment, wherein the internal communication unit comprises a coil or a high-sensitivity magnetic field detector for communicating with the external device.
24. The implant according to any preceding embodiment, further comprising:
a sensation generator configured to generate a sensation detectable by a sense of the patient, the sensation generator being communicatively coupled to the internal control unit and being configured to, upon request, generate the sensation when the implant is implanted in the patient.
25. The implant according to embodiment 24, wherein the sensation generator is configured to receive the request from the internal control unit of the implant.
26. The implant according to any of embodiments 24-25, wherein the sensation generator is configured to create the sensation or sensation components by at least one of
a vibration of the sensation generator;
producing a sound;
providing a photonic signal;
providing a light signal;
providing an electric signal; and
a heat signal.
27. The implant according to any of embodiments 24-26, wherein the sensation generator is configured to be implanted in the patient.
28. The implant according to any of embodiments 24-27, wherein the sensation generator is configured to be worn in contact with the skin of the patient.
29. the implant according to any of embodiments 24-27, the sensation generator is configured generate the sensation without being in physical contact with the patient.
30. A method for programming an implant by an external device, implant comprising an internal computing unit configured to control a function of said implant and an internal memory configured to store: a first control program for controlling the internal computing unit, a second, updatable or configurable, control program for controlling said function of said implant, and a set of predefined program steps for updating the second control program, the external device being configured to communicate with the implant via a first connection, comprising:
providing, at the internal computing unit, a set of predefined program steps for updating the second control program;
transmitting, by the external device, an update comprising a subset of the predefined program steps over the first connection;
receiving, at the internal computing unit, the update,
verifying, by the internal computing unit, that the update comprises a subset of the predefined program steps, and
upon verification of the instructions, running the update at the implant.
31. The method according to embodiment 30, wherein the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.
32. The method according to any of embodiments 30-31, wherein the verifying comprises rejecting the update in response to the update comprising program steps not comprised in the set of predefined program steps.
33. The method according to any of embodiments 30-32, wherein the verifying comprises allowing the update in response to the update only comprising program steps comprised in the set of predefined program steps.
34. The method according to any of embodiments 30-33, further comprising:
authenticating the communication between the implant and the external device over a second connection.
35. The method according to any of embodiments 30-34, wherein the second connection is a wireless short-range connection.
36. The method according to any of embodiments 34-35, wherein the second connection is an electrical connection using the patient's body as a conductor.
37. The method according to any of embodiments 30-36, further comprising, upon verification, installing the update.
38. The method according to any of embodiments 30-36, further comprising:
monitoring for signals by a sensor connected to the internal computing unit;
providing, from a signal provider comprised in the external control unit, a wake signal;
setting, by the internal computing unit and in response to a detected wake signal, a mode of a portion of the internal control unit from a sleep mode to an active mode.
39. The method according to embodiment 38, wherein the portion of the internal computing unit is the first control program or the second control program.
40. The method according to any of embodiments 38-39, further comprising
detecting, using a frequency detector, a frequency for the first communication channel between a first communication unit and a second communication unit, the first communication unit being associated with the internal control unit and the second communication unit being associated with the external device,
wherein the frequency detector is communicatively coupled to the internal computing unit.
41. The method according to embodiment 40, further comprising:
   determining, using the frequency detector, the frequency for the first communication channel.
42. The method according to embodiment 40, further comprising:
generating, using a sensation generator communicatively coupled to the internal control unit, a sensation detectable by a sense of the patient in response to verifying the update, in response to running the update or in response to the update being installed at the implant.
43. The method according to embodiment 42, wherein the generating comprises at least one of:
Providing a vibration of the sensation generator;
producing a sound;
providing a photonic signal;
providing a light signal;
providing an electric signal; and
providing a heat signal.
44. A computer program product comprising a computer-readable storage medium with instructions adapted to carry out the method of any one of embodiments 30-43 and/or with instructions adapted to carry out an action in any of the implant embodiments 1-29, when executed by a computing unit in an external device having processing capability.
45. A computer program product comprising a computer-readable storage medium with instructions adapted to carry out the method of any one of embodiments 30-43 and/or with instructions adapted to carry out an action in any of the implant embodiments 1-29, when executed by a computing unit in the implant having processing capability.
46. The implant according to any one of the following, alone or in any combination; implant embodiments 1-29, with ability to perform method embodiments 30-43, and ability to use program product embodiments 44-45, comprising an internal control unit adapted to be involved in at least a part of the actions performed by the implant in at least a part of any one of the embodiments 1-45 above.

### Aspect 312D eHealth Reprogramming of the Preprogrammed Steps, Embodiments 1

1. An implant comprising:
an internal computing unit configured to control a function of said implant, said internal computing unit comprises an internal memory configured to store:
i. a first control program for controlling the internal computing unit, and
ii. a second, configurable or updatable or programmable, of predefined program steps, control program for controlling said function of said implant,
iii. a set of predefined program steps for selecting predefined program steps of the second control program,
an internal communication unit connected to said internal computing unit and configured to communicate with an external device, a third device, or an external device receiving a data from a third device, wherein said internal computing unit is configured to receive an update and change to reprogram the preprogrammed steps of the second control program via said internal communication unit, and
a verification unit or function of, connected to, or transmitted to said internal computing unit, said verification unit or function being configured to verify that the received update and reprograming with change of the preprogrammed steps to the second control program, comprises program steps comprised in the set of program limitation stored in the internal memory.
2. The implant according to any preceding embodiment, wherein the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.
3. The implant according to any preceding embodiment, wherein the verification function is configured to reject the update in response to the update comprising program steps not comprised in the set of predefined program steps.
4. The implant according to any preceding embodiment, wherein the verification function is configured to allow the update in response to the update only comprising program steps comprised in the set of predefined program steps.
5. The implant according to embodiment 1, wherein the internal communication unit is configured to communicate with the external device via a first wireless connection for receiving the update to the second control program, and a second connection for performing an authentication of the communication with the external device.
6. The implant according to embodiment 5, wherein the second connection is a wireless short-range connection.
7. The implant according to embodiment 5 or 6, wherein the authentication second connection is an electrical connection using the patient's body as a conductor
8. The implant according to any preceding embodiment, wherein the internal computing unit is further configured to, upon verification, installing the update.
9. The implant according to any preceding embodiment, wherein the internal computing unit has a sleep mode and an active mode, and the implant further comprises a sensor configured to detect a wake signal, and wherein the implant is configured to in response to a detected wake signal set the internal computing unit to the active mode.
10. The implant according to embodiment 9, wherein sensor is configured to detect an acoustic signal as wake signal or wherein the sensor is configured to detect a magnetic signal as the wake signal
11. The implant according to any of embodiments 9-10, wherein
the sensor is configured to detect the received signal strength of a signal; and
the implant is further configured to set the internal computing unit to the active mode in response to the sensor detecting a signal exceeding a threshold signal strength.
12. The implant according to any of embodiments 9-11, further comprising a second internal computing unit, and wherein the implant is configured to set the internal computing unit to the active mode via the second internal computing unit.
13. The implant according to any of embodiments 9-12, wherein the internal computing unit in the sleep mode is substantially without power, and wherein setting the internal computing unit in the active mode comprises providing the internal computing unit with power.
14. The implant according to embodiment 13, wherein the implant comprises an energy controller for controlling the power supplied to the internal computing unit.
15. The implant according to embodiment 14, wherein the sensor is configured to provide the energy controller with a second wake signal in response to detecting the wake signal, and wherein the energy controller is configured to set the computing unit in the active mode in response to the second wake signal.
16. The implant according to any preceding embodiment, wherein
the sensor is configured to detect the received signal strength of a signal; and
the internal control unit is further configured to set the internal computing unit to the active mode in response to the sensor detecting a signal exceeding a threshold signal strength.
17. The implant according to any preceding embodiment, wherein
the wake signal comprises a predetermined signal pattern; and
the implant is further configured to set the processing unit to the active mode in response to the sensor detecting the predetermined signal pattern.
18. The implant according to any preceding embodiment, wherein the sensor is a hall effect sensor, a fluxgate sensor, an ultra-sensitive magnetic field sensor or a magneto-resistive sensor.
19. The implant according to any preceding embodiment, wherein the sensor comprises a third coil having an iron core.
20. The implant according to any preceding embodiment, wherein the sensor is comprised in the internal communication unit.
21. The implant according to any preceding embodiment, further comprising a frequency detector, communicatively coupled to the internal computing unit and configured to detect a frequency for data communication between the internal communication unit and an external device configured to transmit a frequency indicator signal.
22. The implant according to embodiment 21, wherein the frequency detector comprises an antenna.
23. The implant according to any preceding embodiment, wherein the internal communication unit comprises a coil or a high-sensitivity magnetic field detector for communicating with the external device.
24. The implant according to any preceding embodiment, further comprising: a sensation generator configured to generate a sensation detectable by a sense of the patient, the sensation generator being communicatively coupled to the internal control unit and being configured to, upon request, generate the sensation when the implant is implanted in the patient.
25. The implant according to embodiment 24, wherein the sensation generator is configured to receive the request from the internal control unit of the implant.
26. The implant according to any of embodiments 24-25, wherein the sensation generator is configured to create the sensation or sensation components by at least one of:
a vibration of the sensation generator;
producing a sound;
providing a photonic signal;
providing a light signal;
providing an electric signal; and
a heat signal.
27. The implant according to any of embodiments 24-26, wherein the sensation generator is configured to be implanted in the patient.
28. The implant according to any of embodiments 24-27, wherein the sensation generator is configured to be worn in contact with the skin of the patient.
29. the implant according to any of embodiments 24-27, the sensation generator is configured generate the sensation without being in physical contact with the patient.
30. A method for programming an implant by an external device, implant comprising an internal computing unit configured to control a function of said implant and an internal memory configured to store: a first control program for controlling the internal computing unit, a second, updatable or configurable, control program for controlling said function of said implant, and a set of predefined program steps for updating the second control program, the external device being configured to communicate with the implant via a first connection, comprising:
providing, at the internal computing unit, a set of predefined program steps for updating the second control program;
transmitting, by the external device, an update comprising a subset of the predefined program steps over the first connection;
receiving, at the internal computing unit, the update,
verifying, by the internal computing unit, that the update comprises a subset of the predefined program steps, and
upon verification of the instructions, running the update at the implant.
31. The method according to embodiment 30, wherein the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.
32. The method according to any of embodiments 30-31, wherein the verifying comprises rejecting the update in response to the update comprising program steps not comprised in the set of predefined program steps.
33. The method according to any of embodiments 30-32, wherein the verifying comprises allowing the update in response to the update only comprising program steps comprised in the set of predefined program steps.
34. The method according to any of embodiments 30-33, further comprising: authenticating the communication between the implant and the external device over a second connection.
35. The method according to any of embodiments 30-34, wherein the second connection is a wireless short-range connection.
36. The method according to any of embodiments 34-35, wherein the second connection is an electrical connection using the patient's body as a conductor.
37. The method according to any of embodiments 30-36, further comprising, upon verification, installing the update.
38. The method according to any of embodiments 30-36, further comprising:
monitoring for signals by a sensor connected to the internal computing unit;
providing, from a signal provider comprised in the external control unit, a wake signal;
setting, by the internal computing unit and in response to a detected wake signal, a mode of a portion of the internal control unit from a sleep mode to an active mode.
39. The method according to embodiment 38, wherein the portion of the internal computing unit is the first control program or the second control program.
40. The method according to any of embodiments 38-39, further comprising
detecting, using a frequency detector, a frequency for the first communication channel between a first communication unit and a second communication unit, the first communication unit being associated with the internal control unit and the second communication unit being associated with the external device,
wherein the frequency detector is communicatively coupled to the internal computing unit.
41. The method according to embodiment 40, further comprising:
   determining, using the frequency detector, the frequency for the first communication channel.
42. The method according to embodiment 40, further comprising:
generating, using a sensation generator communicatively coupled to the internal control unit, a sensation detectable by a sense of the patient in response to verifying the update, in response to running the update or in response to the update being installed at the implant.
43. The method according to embodiment 42, wherein the generating comprises at least one of:
Providing a vibration of the sensation generator;
producing a sound;
providing a photonic signal;
providing a light signal;
providing an electric signal; and
providing a heat signal.
44. A computer program product comprising a computer-readable storage medium with instructions adapted to carry out the method of any one of embodiments 30-43 and/or with instructions adapted to carry out an action in any of the implant embodiments 1-29, when executed by a computing unit in an external device having processing capability.
45. A computer program product comprising a computer-readable storage medium with instructions adapted to carry out the method of any one of embodiments 30-43 and/or with instructions adapted to carry out an action in any of the implant embodiments 1-29, when executed by a computing unit in the implant having processing capability.
46. The implant according to any one of the following, alone or in any combination; implant embodiments 1-29, with ability to perform method embodiments 30-43, and ability to use program product embodiments 44-45, comprising an internal control unit adapted to be involved in at least a part of the actions performed by the implant in at least a part of any one of the embodiments 1-45 above.

### Aspect 316C eHealth Relay Instructions, Embodiments 1-25

1. A method for transmitting an instruction from a first external device to an implant, comprising:
transmitting an instruction for the implant from the first external device to a second external device, the instruction relating to a function of the implant,
encrypting, at the first external device and using a first encryption key, the instruction into an encrypted instruction, and
transmitting the encrypted instruction from the first external device to the second external device, wherein the second external device is relaying the instruction without decrypting the instruction to the implant,
decrypting, at the implant, the instructions using a second authentication, encryption, or authentication and encryption key corresponding or relating to the first encryption key.
2. The method according to embodiment 1, wherein the transmitting of the encrypted instruction from the second external device to the implant comprises:
transmitting the encrypted instruction from the second external device to the first external device, and transmitting the encrypted instruction from the first external device to the implant.
3. The method according to embodiment 1, wherein the transmitting of the encrypted instruction from the second external device to the implant comprises:
transmitting the encrypted instruction from the second external device to a third external device, and transmitting the encrypted instruction from the third external device to the implant.
4. The method according to any preceding embodiment, wherein the second external device is an encryption device communicatively coupled to the first external device, and wherein the communication of the instruction between the second external device and the implant is relayed through the first external device.
5. The method according to any preceding embodiment, further comprising, at the implant, running the instruction.
6. The method according to any preceding embodiment, further comprising receiving, at the first external device, the instruction.
7. The method according to embodiment 6, further comprising displaying, at the external device, a user interface for receiving the instruction.
8. The method according to embodiment 7, wherein the implant comprises a set of a predefined program steps, and wherein the method further comprises verifying, by the implant, that the received instruction is comprised in the predefined program steps.
9. The method according to embodiment 8, wherein the verifying comprises rejecting the instruction in response to the instruction not being comprised in the set of predefined program steps.
10. The method according to any of embodiments 8-9, wherein the verifying comprises allowing the instruction in response to the instruction being comprised in the set of predefined program steps.
11. The method according to any preceding embodiment, wherein the first external device and the implant are configured to communicate over a wireless connection.
12. The method according to embodiment 11, wherein the wireless connection comprises at least one of the following protocols:
Radio Frequency type protocol
RFID type protocol
WLAN type protocol
Bluetooth type protocol
BLE type protocol
NFC type protocol
3G/4G/5G type protocol
GSM type protocol
Bluetooth 5.
13. The method according to any preceding embodiment, wherein the transmitting of data between the first external device and the second external device is performed a wireless connection.
14. The method according to any preceding embodiment, further comprising authenticating the connection between the first external device and the implant over which the encrypted instruction is to be transmitted.
15. The method according to any preceding embodiment, wherein the implant comprises an internal control unit for controlling a function of the implant, and wherein the internal control unit is configured to run the instruction.
16. A system for transmitting an instruction from a first external device to an implant, comprising:
   an implant implanted in a human patient, the implant comprising an internal control unit configured to control a function of the implant and configured to receive an instruction from an external device;
   a first external device configured to receive or determine an instruction to be transmitted to the implant, and to transmit the instruction to a second external device; and
   a second external device configured to receive the instruction transmitted from the first external device, encrypt the instruction, and transmit the encrypted instruction to the implant,
   wherein the implant is configured to received and decrypt the instruction.
17. The system according to embodiment 16, wherein the second external device is configured to transmit the encrypted instruction by transmitting the encrypted instruction to the first external device, and wherein the first external device is configured to transmit the encrypted instruction to the implant.
18. The system according to embodiment 16, wherein the second external device is configured to transmit the encrypted instruction by transmitting the encrypted instruction to a third external device, and
wherein the third external device is configured to transmit the encrypted instruction to the implant.
19. The system according to any of embodiments 16-18, wherein the second external device is an encryption device communicatively coupled to the first external device, and wherein any communication between the implant and the second external device is relayed through the first external device.
20. The system according to any one of embodiments 16-19, wherein the internal control unit is configured to run the decrypted instruction for controlling a function of the implant.
21. The system according to any one of embodiments 16-20, wherein the first external device is configured to display a user interface for receiving the instruction.
22. The system according to any one of embodiments 16-21, wherein the implant comprises a set of a predefined program steps, and wherein the implant is configured to verify that the received instruction is comprised in the predefined program steps.
23. The system according to embodiment 22, wherein the implant is configured to reject the instruction in response to the instruction not being comprised in the set of predefined program steps.
24. The system according to any of embodiments 21-22, wherein the implant is configured to allow the instruction in response to the instruction being comprised in the set of predefined program steps.
25. The system according to any of embodiments 16-24, wherein the first external device and the implant are configured to communicate over a wireless connection.
26. A computer program product comprising a computer-readable storage medium with instructions adapted to carry out the method of any one of embodiments 1-15 and/or with instructions adapted to carry out an action in any of the system embodiments 16-25, when executed by a computing unit in an external device having processing capability.
27. A computer program product comprising a computer-readable storage medium with instructions adapted to carry out the method of any one of embodiments 1-15 and/or with instructions adapted to carry out an action in any of the system embodiments 16-25, when executed by a computing unit in the implant having processing capability.

### Aspect 317C Energy General Microphone, Embodiments 1-19

1. An implantable controller for controlling an energized implant, when implanted in a patient, the controller comprises:
   a computing unit,
at least one microphone, at least one accelerometer, or at least one microphone and at least one accelerometer, wherein the at least one microphone, at least one accelerometer, or at least one microphone and at least one accelerometer is configured to register a sound, a vibration or a movement related to at least one of: a bodily function, and a function of the implant.
2. The implantable controller according to embodiment 1, wherein the implantable controller further comprises at least one implantable housing for sealing against fluid, and wherein the computing unit and the microphone are placed inside of the housing.
3. The implantable controller according to any one of embodiments 1 and 2, wherein the computing unit is configured to derive a pulse of the patient from the registered sound related to a bodily function.
4. The implantable controller according to any one of embodiments 1 and 2, wherein the computing unit is configured to derive information related to the patient urinating from the registered sound related to a bodily function.
5. The implantable controller according to any one of embodiments 1 and 2, wherein the computing unit is configured to derive information related to a bowel activity of the patient from the registered sound related to a bodily function.
6. The implantable controller according to any one of embodiments 1 and 2, wherein the computing unit is configured to derive information related to a functional status of the implant from the registered sound related to a function of the implant.
7. The implantable controller according to embodiment 6, wherein the computing unit is configured to derive information related to the functional status of an operation device of the implant, from the registered sound related to a function of the implant.
8. The implantable controller according to embodiment 7, wherein the computing unit is configured to derive information related to the functional status of at least one of: a motor, a pump and a transmission of the operation device of the implant from, the registered sound related to a function of the implant.
9. The implantable controller according to any one of the preceding embodiments, further comprising a transceiver, and wherein the controller is configured to transmit a parameter derived from the sound registered by the at least one microphone using the transceiver.
10. A method of authenticating at least one of an energized implant implanted in a patent, an external device, and a connection between the energized implant and the external device performed in a system comprising the energized implant and the external device, the energized implant comprising at least one microphone, and a transmitter, and the external device comprising a receiver and a computing unit, the method comprising:
registering a sound related to at least one of: a bodily function and a function of the implant, using the at least one microphone,
transmitting a signal derived from the registered sound, using the transmitter,
receiving, in the external device, the signal derived from the registered sound, using the receiver, and
comparing, in the external device, a parameter derived from the received signal with a reference parameter, using the computing unit.
11. The method according to embodiment 9, further comprising the step of authenticating at least one of:
the energized implant,
the external device, and
the connection between the energized implant and the external device the energized implant on the basis of the comparison.
12. The method according to any one of embodiments 10 and 11, further comprising receiving, at the receiver of the external device, a parameter to be used as reference parameter.
13. The method according to embodiment 12, wherein the step of receiving a parameter to be used as reference parameter comprises receiving the parameter from a sensor external to the patient.
14. The method according to any one of embodiments 10-13, wherein the registered sound is related to a pulse of the patient, and wherein the reference parameter is related to the pulse of the patient.
15. A method of authenticating at least one of an energized implant implanted in a patent, an external device, and a connection between the energized implant and the external device, performed in a system comprising the energized implant and the external device, the energized implant comprising at least one microphone, a receiver, and a computing unit, and the external device comprising a transmitter, the method comprising:
registering a sound related to at least one of: a bodily function and a function of the implant, using the at least one microphone,
deriving a parameter from the sound using the computing unit,
receiving, in the energized implant, a reference parameter, from the external device, using the receiver, and
comparing, in the energized implant, the parameter derived from the sound with the received reference parameter, using the computing unit.
16. The method according to embodiment 15, further comprising the step of authenticating at least one of:
the energized implant,
the external device, and
the connection between the energized implant and the external device on the basis of the comparison.
17. The method according to any one of embodiments 15 and 16, further comprising receiving, at a receiver of the external device, a parameter to be used as reference parameter.
18. The method according to embodiment 17, wherein the step of receiving a parameter to be used as reference parameter comprises receiving the parameter from a sensor external to the patient.
19. The method according to any one of embodiments 15-18, wherein the registered sound is related to a pulse of the patient, and wherein the reference parameter is related to the pulse of the patient.

### Aspect 384SE—eHealth_General_Communication_Dual, Embodiments 1-24

1. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device.
2. The external device according to embodiment 1, wherein the first wireless transceiver comprises an UWB transceiver.
3. The external device according to embodiment 1, wherein the first wireless transceiver is configured for transcutaneous energy transfer for at least one of:
   powering an energy consuming component of the implantable medical device, and charging an implantable energy storage unit.
4. The external device according to any one of the preceding embodiments, wherein the second network protocol is a standard network protocol.
5. The external device according to any one of the preceding embodiments, wherein the second wireless transceiver comprises a Bluetooth transceiver.
6. The external device according to any one of the preceding embodiments, wherein the external device is further configured to communicate with a second external device using said at least one wireless transceiver.
7. The external device according to any one of the preceding embodiments, wherein the external device is configured for determining a distance between the external device and the implantable medical device by determining the RSSI.
8. The external device according to any one of embodiments 4-7, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
9. The external device according to any one of the preceding embodiments, wherein a communication range of the first network protocol is less than a communication range of the second network protocol.
10. The external device according to any one of the preceding embodiments, wherein a frequency band of the first network protocol differs from a frequency band of the second network protocol.
11. The external device according to any one of the preceding embodiments, wherein the external device is configured to authenticate the implantable medical device if the determined distance between the external device and the implantable medical device is less than a predetermined threshold value.
12. The external device according to embodiment 11, wherein the external device is configured to allow the transfer of data between the external device and the implantable medical device after the implantable medical device has been authenticated.
13. The external device according to any one of the preceding embodiments, wherein the external device is one from the list of:
   a wearable external device, and
   a handset.
14. An implantable medical device configured for communication with an external device, the implantable medical device comprising:
   at least one first wireless transceiver configured for communication with the external device using a first network protocol, for determining a distance between the external device and the implantable medical device, and
   at least one second wireless transceiver configured for communication with the external device using a second network protocol, for transferring data between the external device and the implantable medical device.
15. The implantable medical device according to embodiment 14, wherein the first wireless transceiver comprises an UWB transceiver.
16. The implantable medical device according to embodiment 14, wherein the first wireless transceiver is configured for transcutaneous energy transfer for at least one of:
   powering an energy consuming component of the implantable medical device, and
   charging an implantable energy storage unit.
17. The implantable medical device according to any one of embodiments 14-16, wherein the second network protocol is a standard network protocol.
18. The implantable medical device according to any one of embodiments 14-17, wherein the second wireless transceiver comprises a Bluetooth transceiver.
19. The implantable medical device according to any one of embodiments 14-18, wherein the implantable medical device is further configured to communicate with a second external device using said at least one wireless transceiver.
20. The implantable medical device according to any one of embodiments 14-19, wherein the implantable medical device is configured for determining a distance between the external device and the implantable medical device by determining the RSSI.
21. The implantable medical device according to any one of embodiments 14-20, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
22. The implantable medical device according to any one of the embodiments 14-21, wherein a communication range of the first network protocol is less than a communication range of the second network protocol.
23. The implantable medical device according to any one of embodiments 14-22, wherein a frequency band of the first network protocol differs from a frequency band of the second network protocol.
24. The implantable medical device according to any one of embodiments 14-23, wherein the implantable medical device is configured to authenticate the external device if the determined distance between the external device and the implantable medical device is less than a predetermined threshold value.
25. The implantable medical device according to embodiment 24, wherein the implantable medical device is configured to allow the transfer of data between the implantable medical device and the external device after the external device has been authenticated.
26. The implantable medical device according to any one of embodiments 14-25, wherein the implantable medical device comprises at least one of
   an external heart compression device,
   an apparatus assisting the pump function of a heart of the patient,
   an apparatus assisting the pump function comprising a turbine bump placed within a patient's blood vessel for assisting the pump function of the heart,
   an operable artificial heart valve,
   an operable artificial heart valve for increasing the blood flow to the coronary arteries.
   an implantable drug delivery device,
   an implantable drug delivery device for injecting directly into a blood vessel and change the position of the injection site, all from within the patient's body,
   an implantable drug delivery device for injecting potency enhancing drugs into an erectile tissue of the patient,
   a hydraulic, mechanic, and/or electric constriction implant,
   an operable volume filling device,
   an operable gastric band,
   an operable implant for stretching the stomach wall of the patient for creating satiety,
   an implant configured to sense the frequency of the patient ingesting food,
   an operable cosmetic implant,
   an operable cosmetic implant for adjust the shape and/or size in the breast region of a patient,
   an implant controlling medical device for the emptying of a urinary bladder,
   an implant hindering urinary leakage,
   an implant hindering anal incontinence,
   an implant controlling the emptying of fecal matter,
   an implant monitoring an aneurysm,
   an implant for hindering the expansion of an aneurysm,
   an implant lubricating a joint,
   an implant for affecting the blood flow to an erectile tissue of the patient,
   an implant for simulating the engorgement of an erectile tissue,
   an implant with a reservoir for holding bodily fluids,
   an implant storing and/or emptying a bodily reservoir or a surgically created reservoir,
   an implant communicating with a database outside the body,
   an implant able to be programmed from outside the body,
   an implant able to be programmed from outside the body with a wireless signal,
   an implant treating impotence,
   an implant controlling the flow of eggs in the uterine tube,
   an implant controlling the flow of sperms in the uterine tube,
   an implant controlling the flow of sperms in the vas deferens,
   an implant for hindering the transportation of the sperm in the vas deferens,
   an implant treating osteoarthritis,
   an implant performing a test of parameters inside the body,
   an implant controlling specific treatment parameters from inside the body,
   an implant controlling bodily parameters from inside the body,
   an implant controlling the blood pressure,
   an implant controlling the blood pressure by affecting the dilatation of the renal artery,
   an implant controlling a drug treatment parameter,
   an implant controlling a parameter in the blood,
   an implant for adjusting or replacing any bone part of a body of the patient,
   an implant replacing an organ of the patient or part of an organ of the patient or the function thereof,
   a vascular treatment device,
   an implant adapted to move fluid inside the body of the patient,
   an implant configured to sense a parameter related to the patient swallowing,
   an implant configured to exercise a muscle with electrical or mechanical stimulation,
   an implant configured for emptying an intestine portion on command,
   an operable implant configured to be invaginated in the stomach of the patient to reduce the volume of the stomach substantially more than the volume of the device,
   an implant configured for emptying the urinary bladder from within the patient's body by compressing the bladder,
   an implant configured for draining fluid from within the patient's body,
   an implant configured for the active lubrication of a joint with an added lubrication fluid,
   an implant configured for removing clots and particles from the patient's blood stream,
   an implant configured for elongating or straightening a bone in the patient, to reduce scoliosis,
   a device to stimulate the brain for a several position to a focused point,
   an artificial stomach replacing the function of the natural stomach,
   an implant configured for adjusting the position of a female's urinary tract or bladder neck,
   an implant configured for stimulating the ampulla vas deference and creating temporary constriction.

### Aspect 384 B—eHealth_General_Communication_Dual, Embodiments 1-24

1. A system comprising
an implantable medical device;
an external device configured for communication with the implantable medical device when implanted in a patient, the external device comprising:
at least one first wireless transceiver configured for communication with the implantable medical device using a first network protocol, for determining a distance between the external device and the implantable medical device, and
at least one second wireless transceiver configured for communication with the implantable medical device using a second network protocol, for transferring data between the external device and the implantable medical device, wherein
the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device, wherein
   the implant comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a system for treating a female patient to avoid or promote pregnancy comprising a restriction device adapted to postoperatively restrict and release an oviduct of the patient.
18. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 67-71, wherein the first wireless transceiver comprises an UWB transceiver.
23. The system according to any one of embodiments 1-21 or 67-71, wherein the first wireless transceiver is configured for transcutaneous energy transfer for at least one of: powering an energy consuming component of the implantable medical device, and charging an implantable energy storage unit.
24. The system according to any one of the preceding embodiments or 67-71, wherein the second network protocol is a standard network protocol.
25. The system according to any one of the preceding embodiments or 67-71, wherein the second wireless transceiver comprises a Bluetooth transceiver.
26. The system according to any one of the preceding embodiments or 67-71, wherein the external device is further configured to communicate with a second external device using said at least one wireless transceiver.
27. The system according to any one of the preceding embodiments or 67-71, wherein the external device is configured for determining a distance between the external device and the implantable medical device by determining the RSSI.
28. The system according to any one of embodiments 24-27, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
29. The system according to any one of the preceding embodiments or 67-71, wherein a communication range of the first network protocol is less than a communication range of the second network protocol.
30. The system according to any one of the preceding embodiments or 67-71, wherein a frequency band of the first network protocol differs from a frequency band of the second network protocol.
31. The system according to any one of the preceding embodiments or 67-71, wherein the external device is configured to authenticate the implantable medical device if the determined distance between the external device and the implantable medical device is less than a predetermined threshold value.
32. The system according to embodiment 31, wherein the external device is configured to allow the transfer of data between the external device and the implantable medical device after the implantable medical device has been authenticated.
33. The system according to any one of the preceding embodiments or 67-71, wherein the external device is one from the list of:
   a wearable external device, and
   a handset.
34. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
35. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
36. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
37. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
38. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
39. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
40. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
41. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
42. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an apparatus for drainage of a body fluid or movement of hydraulic treatment fluid in a human or mammal patient comprising a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
43. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
44. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
45. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
46. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
47. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
48. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
49. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
50. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a system for treating a female patient to avoid or promote pregnancy comprising a restriction device adapted to postoperatively restrict and release an oviduct of the patient.
51. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
52. An implant comprising:
at least one sensor for sensing at least one physiological parameter of the patient or a functional parameter of the implant to obtain a sensed parameter, and
a communication unit configured to broadcast data;
wherein the sensor is configured to periodically sense the parameter and wherein the communication unit is configured to broadcast the data relating to the sensed parameter in response to at least one of
the sensed parameter being above a predetermined threshold,
the sensed parameter being below a predetermined threshold,
the sensed parameter being outside of a predetermined range,
a predetermined point in time,
an expiry of a time period,
a predetermined event, or
a use of the implant, wherein
the implant comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
53. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
54. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
55. The implantable medical device according to any one of embodiments 34-54 or 71-75,
wherein the first wireless transceiver comprises an UWB transceiver.
56. The implantable medical device according to any one of embodiments 34-54 or 71-75,
wherein the first wireless transceiver is configured for transcutaneous energy transfer for at least one of: powering an energy consuming component of the implantable medical device, and charging an implantable energy storage unit.
57. The implantable medical device according to any one of embodiments 34-56 or 71-75,
wherein the second network protocol is a standard network protocol.
58. The implantable medical device according to any one of embodiments 34-57 or 71-75,
wherein the second wireless transceiver comprises a Bluetooth transceiver.
59. The implantable medical device according to any one of embodiments 34-58 or 71-75,
wherein the implantable medical device is further configured to communicate with a second external device using said at least one wireless transceiver.
60. The implantable medical device according to any one of embodiments 34-59 or 71-75,
wherein the implantable medical device is configured for determining a distance between the external device and the implantable medical device by determining the RSSI.
61. The implantable medical device according to any one of embodiments 34-60 or 71-75,
wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
62. The implantable medical device according to any one of the embodiments 34-61 or 71-75, wherein a communication range of the first network protocol is less than a communication range of the second network protocol.
63. The implantable medical device according to any one of embodiments 34-62 or 71-75,
wherein a frequency band of the first network protocol differs from a frequency band of the second network protocol.
64. The implantable medical device according to any one of embodiments 34-63 or 71-75,
wherein the implantable medical device is configured to authenticate the external device if the determined distance between the external device and the implantable medical device is less than a predetermined threshold value.
65. The implantable medical device according to embodiment 64, wherein the implantable medical device is configured to allow the transfer of data between the implantable medical device and the external device after the external device has been authenticated.
66. A system comprising
an implantable medical device;
an external device configured for communication with the implantable medical device when implanted in a patient, the external device comprising:
at least one first wireless transceiver configured for communication with the implantable medical device using a first network protocol, for determining a distance between the external device and the implantable medical device, and
at least one second wireless transceiver configured for communication with the implantable medical device using a second network protocol, for transferring data between the external device and the implantable medical device, wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
67. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a device for treating obesity of a patient, the device comprising
      at least one operable stretching device (10; 110) implantable in the patient and adapted to stretch a portion of the patient's stomach wall (12), and
      an implantable control unit (42) for automatically controlling the operable stretching device, when the control unit and stretching device are implanted, to stretch the stomach wall portion in connection with the patient eating such that satiety is created,
      wherein the stretching device comprises a first and a second engaging part (10″; 110a; 110b; 210a; 210b), wherein:
      the first part is adapted to be engaged to a first area of the stomach wall, and
      the second part is adapted to be engaged to a second area of the stomach wall, and wherein
      the stretching device is adapted to stretch a portion of the stomach wall between the first area and the second area.
68. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises an obesity treatment device comprising:
      at least one operable stretching device (10; 10″; 110a; 110b; 410b; 50; 810) implantable in a patient and adapted to stretch a portion of the patient's stomach wall (12) and
      an implantable operation device (90; 42; 54; 40; 217; 218; 452) for operating the stretching device, when implanted, to stretch the stomach wall portion such that satiety is created,
      wherein the operable stretching device is adapted to be placed against the outside of the stomach wall.
69. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a device for treating obesity of a patient, the device comprising:
      at least one operable stretching device implantable in the patient and adapted to stretch a portion of the patient's stomach wall, and an implantable control unit for controlling the operable stretching device, when the control unit and the stretching device are implanted, to stretch the stomach wall portion in connection with the patient eating such that satiety is created,
      wherein the stretching device comprises a first engaging part and a second engaging part, wherein:
      the first engaging part is adapted to be engaged to a first area of the stomach wall, and
      the second engaging part is adapted to be engaged to a second area of the stomach wall, and wherein
      the stretching device is adapted to stretch a portion of the stomach wall between the first area and the second area.
70. A system comprising
   an implantable medical device;
   an external device configured for communication with the implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.
71. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
72. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
73. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
74. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
75. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device, wherein
the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 385SE—eHealth_General_General_Remote Display Portal, Embodiments 1-35

1. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device.
2. The patient external device according to embodiment 1, wherein the wireless communication unit comprises a wireless transceiver for:
   wireless transmission of control commands to the implantable medical device, and
   wireless transmission of the control interface as the remote display portal to the patient display device.
3. The patient external device according to embodiment 1, wherein the wireless communication unit comprises:
   a first wireless transceiver for wireless transmission of control commands to the implantable medical device, and
   a second wireless transceiver for wireless transmission of the control interface to the patient display device.
4. The patient external device according to any one of the preceding embodiments, wherein the wireless communication unit is configured for wireless communication with the patient display device using a standard network protocol.
5. The patient external device according to any one of the preceding embodiments, wherein the wireless communication unit is configured for wireless communication with the implantable medical device using a proprietary network protocol.
6. The patient external device according to any one of the preceding embodiments, wherein the wireless communication unit comprises a Bluetooth transceiver.
7. The patient external device according to embodiment 6, wherein at least one of the first and second wireless transceiver comprises a Bluetooth transceiver.
8. The patient external device according to any one of embodiments 1-5, wherein the wireless communication unit comprises a UWB transceiver.
9. The patient external device according to embodiment 8, wherein at least one of the first and second wireless transceiver comprises a UWB transceiver.
10. The patient external device according to embodiment 1, wherein the wireless communication unit comprises:
   at least one first wireless transceiver configured for communication with the implantable medical device using a first network protocol, for determining a distance between the patient external device and the implantable medical device, and
   at least one second wireless transceiver configured for communication with the implantable medical device using a second network protocol, for transferring data between the patient external device and the implantable medical device.
11. The patient external device according to embodiment 3, wherein the first wireless transceiver is configured for transcutaneous energy transfer for at least one of:
   powering an energy consuming component of the implantable medical device, and
   charging an implantable energy storage unit.
12. The patient external device according to embodiment 4, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
13. The patient external device according to any one of embodiments 3-12, wherein a communication range of the first wireless transceiver is less than a communication range of the second wireless transceiver.
14. The patient external device according to any one of the preceding embodiments, wherein at least one of:
   the patient external device is configured to authenticate the implantable medical device if a distance between the patient external device and the implantable medical device is less than a predetermined threshold value,
   the patient external device is configured to be authenticated by the implantable medical device if a distance between the patient external device and the implantable medical device is less than a predetermined threshold value,
   the patient external device is configured to authenticate the patient display device if a distance between the patient external device and the patient display device is less than a predetermined threshold value, and
   the patient external device is configured to be authenticated by the implantable medical device if a distance between the patient external device and the patient display device is less than a predetermined threshold value.
15. The patient external device according to embodiment 14, wherein the patient external device is configured to allow the transfer of data between at least one of
   the patient external device and the implantable medical device, and
   the patient external device and the patient display device, on the basis of the authentication.
16. The communication system according to any one of the preceding embodiments, wherein the computing unit is configured to encrypt at least one of the control interface and the control commands.
17. The implantable medical device according to any one of preceding embodiments, wherein the implantable medical device comprises at least one of
   an external heart compression device,
   an apparatus assisting the pump function of a heart of the patient,
   an apparatus assisting the pump function comprising a turbine bump placed within a patient's blood vessel for assisting the pump function of the heart,
   an operable artificial heart valve,
   an operable artificial heart valve for increasing the blood flow to the coronary arteries.
   an implantable drug delivery device,
   an implantable drug delivery device for injecting directly into a blood vessel and change the position of the injection site, all from within the patient's body,
   an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
   an implant with a reservoir for holding bodily fluids,
   an implant storing and/or emptying a bodily reservoir or a surgically created reservoir,
   an implant communicating with a database outside the body,
   an implant able to be programmed from outside the body,
   an implant able to be programmed from outside the body with a wireless signal,
   an implant treating impotence,
   an implant controlling the flow of eggs in the uterine tube,
   an implant controlling the flow of sperms in the uterine tube,
   an implant controlling the flow of sperms in the vas deferens,
   an implant for hindering the transportation of the sperm in the vas deferens,
   an implant treating osteoarthritis,
   an implant performing a test of parameters inside the body,
   an implant controlling specific treatment parameters from inside the body,
   an implant controlling bodily parameters from inside the body,
   an implant controlling the blood pressure,
   an implant controlling the blood pressure by affecting the dilatation of the renal artery,
   an implant controlling a drug treatment parameter,
   an implant controlling a parameter in the blood,
   an implant for adjusting or replacing any bone part of a body of the patient,
   an implant replacing an organ of the patient or part of an organ of the patient or the function thereof,
   a vascular treatment device,
   an implant adapted to move fluid inside the body of the patient,
   an implant configured to sense a parameter related to the patient swallowing,
   an implant configured to exercise a muscle with electrical or mechanical stimulation,
   an implant configured for emptying an intestine portion on command,
   an operable implant configured to be invaginated in the stomach of the patient to reduce the volume of the stomach substantially more than the volume of the device,
   an implant configured for emptying the urinary bladder from within the patient's body by compressing the bladder,
   an implant configured for draining fluid from within the patient's body,
   an implant configured for the active lubrication of a joint with an added lubrication fluid,
   an implant configured for removing clots and particles from the patient's blood stream,
   an implant configured for elongating or straightening a bone in the patient, to reduce scoliosis,
   a device to stimulate the brain for a several position to a focused point,
   an artificial stomach replacing the function of the natural stomach,
   an implant configured for adjusting the position of a female's urinary tract or bladder neck,
   an implant configured for stimulating the ampulla vas deference and creating temporary constriction.
18. A patient display device for communication with a patient remote external device for communication with an implantable medical device, the patient display device comprising:
   a wireless communication unit configured for wirelessly receiving an implant control interface as a remote display portal from the patient remote external device and configured for wirelessly transmitting implant control user input to the patient remote external device,
   a display for displaying the received implant control interface, and
   an input device for receiving implant control input from the user.
19. The patient display device according to embodiment 18 wherein the patient display device further comprises an auxiliary wireless communication unit, and wherein the auxiliary wireless communication unit is configured to be disabled to enable at least one of:
   wirelessly receiving the implant control interface as the remote display portal from the patient remote external device, and wirelessly transmitting implant control user input to the patient remote external device.
20. The patient display device according to any one of embodiments 18-19, wherein the wireless communication unit is configured for wireless communication with the patient remote external device using a standard network protocol.
21. The patient display device according to any one of embodiments 18-20, wherein the wireless communication unit is configured for wireless communication with the patient remote external device using a proprietary network protocol.
22. The patient display device according to any one of embodiments 18-20, wherein the wireless communication unit comprises a Bluetooth transceiver.
23. The patient display device according to any one of embodiments 18-22, wherein the wireless communication unit comprises a UWB transceiver.
24. The patient display device according to embodiment 20, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
25. The patient display device according to embodiment 19, wherein a communication range of the wireless communication unit is less than a communication range of the auxiliary wireless communication unit.
26. The patient display device according to any one of embodiments 18-25, wherein at least one of:
   the patient display device is configured to authenticate the patient remote external device if a distance between the patient display device and the patient remote external device is less than a predetermined threshold value, and
   the patient display device is configured to be authenticated by the patient remote external device if a distance between the patient display device and the patient remote external device is less than a predetermined threshold value.
27. The patient display device according to embodiment 26, wherein the patient display device is configured to allow the transfer of data between the patient display device and the patient remote external device on the basis of the authentication.
28. The patient display device according to any one of the preceding embodiments, wherein the patient display device is a wearable external device or a handset.
29. A communication system for enabling communication between a patient display device and an implantable medical device, when implanted, the communication system comprising:
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device.
30. The communication system according to embodiment 29, wherein the computing unit is configured to encrypt at least one of the control interface and the control commands.
31. The communication system according to any one of embodiments 29 and 30, wherein the patient display device is configured to encrypt the user input.
32. The communication system according to any one of embodiments 29-31, wherein the server is configured to encrypt at least one of the user input received from the patient display device and the control interface received from the patient remote external device.
33. The communication system according to embodiment 29, wherein the computing unit is configured to encrypt the control interface and the patient display device is configured to decrypt the encrypted control interface.
34. The communication system according to embodiment 33, wherein the server is configured to act as a router, transferring the encrypted control interface from the patient remote external device to the patient display device without decryption.
35. The communication system or patient display device according to any one of the preceding embodiments, wherein the implantable medical device comprises at least one of
   an external heart compression device,
   an apparatus assisting the pump function of a heart of the patient,
   an apparatus assisting the pump function comprising a turbine bump placed within a patient's blood vessel for assisting the pump function of the heart,
   an operable artificial heart valve,
   an operable artificial heart valve for increasing the blood flow to the coronary arteries.
   an implantable drug delivery device,
   an implantable drug delivery device for injecting directly into a blood vessel and change the position of the injection site, all from within the patient's body,
   an implantable drug delivery device for injecting potency enhancing drugs into an erectile tissue of the patient,
   a hydraulic, mechanic, and/or electric constriction implant,
   an operable volume filling device,
   an operable gastric band,
   an operable implant for stretching the stomach wall of the patient for creating satiety,
   an implant configured to sense the frequency of the patient ingesting food,
   an operable cosmetic implant,
   an operable cosmetic implant for adjust the shape and/or size in the breast region of a patient,
   an implant controlling medical device for the emptying of a urinary bladder,
   an implant hindering urinary leakage,
   an implant hindering anal incontinence,
   an implant controlling the emptying of fecal matter,
   an implant monitoring an aneurysm,
   an implant for hindering the expansion of an aneurysm,
   an implant lubricating a joint,
   an implant for affecting the blood flow to an erectile tissue of the patient,
   an implant for simulating the engorgement of an erectile tissue,
   an implant with a reservoir for holding bodily fluids,
   an implant storing and/or emptying a bodily reservoir or a surgically created reservoir,
   an implant communicating with a database outside the body,
   an implant able to be programmed from outside the body,
   an implant able to be programmed from outside the body with a wireless signal,
   an implant treating impotence,
   an implant controlling the flow of eggs in the uterine tube,
   an implant controlling the flow of sperms in the uterine tube,
   an implant controlling the flow of sperms in the vas deferens,
   an implant for hindering the transportation of the sperm in the vas deferens,
   an implant treating osteoarthritis,
   an implant performing a test of parameters inside the body,
   an implant controlling specific treatment parameters from inside the body,
   an implant controlling bodily parameters from inside the body,
   an implant controlling the blood pressure,
   an implant controlling the blood pressure by affecting the dilatation of the renal artery,
   an implant controlling a drug treatment parameter,
   an implant controlling a parameter in the blood,
   an implant for adjusting or replacing any bone part of a body of the patient,
   an implant replacing an organ of the patient or part of an organ of the patient or the function thereof,
   a vascular treatment device,
   an implant adapted to move fluid inside the body of the patient,
   an implant configured to sense a parameter related to the patient swallowing,
   an implant configured to exercise a muscle with electrical or mechanical stimulation,
   an implant configured for emptying an intestine portion on command,
   an operable implant configured to be invaginated in the stomach of the patient to reduce the volume of the stomach substantially more than the volume of the device,
   an implant configured for emptying the urinary bladder from within the patient's body by compressing the bladder,
   an implant configured for draining fluid from within the patient's body,
   an implant configured for the active lubrication of a joint with an added lubrication fluid,
   an implant configured for removing clots and particles from the patient's blood stream,
   an implant configured for elongating or straightening a bone in the patient, to reduce scoliosis,
   a device to stimulate the brain for a several position to a focused point,
   an artificial stomach replacing the function of the natural stomach,
   an implant configured for adjusting the position of a female's urinary tract or bladder neck,
   an implant configured for stimulating the ampulla vas deference and creating temporary constriction.
36. The communication system according to any one of the preceding embodiments, further comprising a server comprising:
a wireless communication unit configured for wirelessly receiving
an implant control interface received from the patient remote external device and wirelessly transmitting the implant control interface as a remote display portal to the patient display device, the wireless communication unit further being configured for wirelessly receiving implant control user input from a patient EID external device and wirelessly transmitting the implant control user input to the patient display device.
37. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
38. The system according to anyone of the preceding embodiments, wherein the patient remote external device and the patient EID external device are an integrated unit.
39. The system according to anyone of the preceding embodiments, wherein the HCP dedicated device and the HCP EID external device are an integrated unit.
40. The system according to anyone of the preceding embodiments, comprising a measurement device or sensor adapted to deliver a measurement to at least one of the DDI, patent EID external device and a patient display device.
41. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.

### Aspect 385B—eHealth_General_General_Remote Display Portal, Embodiments 1-35

1. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A communication system for enabling communication between a patient display device and an implantable medical device, when implanted, the communication system comprising:
   an implantable medical device,
   a patient display device,
   a server, and
   a patient remote external device, wherein:
   the patient display device comprises:
   a wireless communication unit configured for wirelessly receiving an implant control interface as a remote display portal being provided by the patient remote external device, the wireless communication unit further being configured for wirelessly transmitting implant control user input to the server, destined for the patient remote external device,
   a display for displaying the received remote display portal, and
   an input device for receiving implant control input from the user,
   the patient remote external device comprising:
   a wireless communication unit configured for wireless transmission of control commands to the implantable medical device, and
   a computing unit configured for:
   running a control software for creating the control commands for the operation of the implantable medical device,
   transmitting a control interface to the patient display device,
   receiving implant control user input generated at the patient display device, from the server, and
   transforming the user input into the control commands for wireless transmission to the implantable medical device, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises a system for treating a female patient to avoid or promote pregnancy comprising a restriction device adapted to postoperatively restrict and release an oviduct of the patient.
18. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The communication system according to any one of embodiments 1-21 or 28-32,
wherein the computing unit is configured to encrypt at least one of the control interface and the control commands.
23. The communication system according to any one of embodiments 1-22 or 28-32,
wherein the patient display device is configured to encrypt the user input.
24. The communication system according to any one of embodiments 1-23 or 28-32,
wherein the server is configured to encrypt at least one of the user input received from the patient display device and the control interface received from the patient remote external device.
25. The communication system according to any one of embodiments 1-21 or 28-32,
wherein the computing unit is configured to encrypt the control interface and the patient display device is configured to decrypt the encrypted control interface.
26. The communication system according to embodiment 25, wherein the server is configured to act as a router, transferring the encrypted control interface from the patient remote external device to the patient display device without decryption.
27. The communication system according to any one of embodiments 1-26 or 28-32, further comprising a server comprising:
a wireless communication unit configured for wirelessly receiving
an implant control interface received from the patient remote external device and wirelessly transmitting the implant control interface as a remote display portal to the patient display device, the wireless communication unit further being configured for wirelessly receiving implant control user input from a patient EID external device and wirelessly transmitting the implant control user input to the patient display device.
28. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
29. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
30. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
31. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
32. A communication system for enabling communication between a patient display device and an implantable medical device, when implanted, the communication system comprising:
an implantable medical device,
a patient display device,
a server, and
a patient remote external device, wherein:
the patient display device comprises:
a wireless communication unit configured for wirelessly receiving an implant control interface as a remote display portal being provided by the patient remote external device, the wireless communication unit further being configured for wirelessly transmitting implant control user input to the server, destined for the patient remote external device,
a display for displaying the received remote display portal, and
an input device for receiving implant control input from the user,
the patient remote external device comprising:
a wireless communication unit configured for wireless transmission of control commands to the implantable medical device, and
a computing unit configured for:
running a control software for creating the control commands for the operation of the implantable medical device,
transmitting a control interface to the patient display device,
receiving implant control user input generated at the patient display device, from the server, and
transforming the user input into the control commands for wireless transmission to the implantable medical device, wherein
the implantable medical device comprises a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 386SE—eHealth_General_General_App_in_App, Embodiments 1-43

1. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application.
2. The patient display device according to embodiment 1, wherein the first log-in is a PIN-based log-in.
3. The patient display device according to embodiment 1, wherein at least one of the first and second log-in is a log-in based on a biometric input or a hardware key.
4. The patient display device according to any one of embodiments 1-3, wherein the patient display device further comprises an auxiliary wireless communication unit, and wherein the auxiliary wireless communication unit is configured to be disabled to enable wireless communication with the patient external device.
5. The patient display device according to any one of embodiments 1-4, wherein the patient display device is configured to wirelessly receive an implant control interface as a remote display portal from the patient external device to be displayed on the display.
6. The patient display device according to any one of embodiments 1-5, wherein the wireless communication unit is configured for wireless communication with the patient external device using a standard network protocol.
7. The patient display device according to any one of embodiments 1-5, wherein the wireless communication unit is configured for wireless communication with the patient external device using a proprietary network protocol.
8. The patient display device according to any one of embodiments 1-5, wherein the wireless communication unit is configured for wireless communication with the patient external device using a first network protocol and with the server using a second network protocol.
9. The patient display device according to any one of embodiments 1-5, wherein the wireless communication unit is configured for wireless communication with the patient external device using a first frequency band and with the server using a second frequency band.
10. The patient display device according to any one of embodiments 1-9, wherein the wireless communication unit comprises a Bluetooth transceiver.
11. The patient display device according to any one of embodiments 1-10, wherein the wireless communication unit comprises a UWB transceiver.
12. The patient display device according to embodiment 6, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
13. The patient display device according to embodiment 4, wherein a communication range of the wireless communication unit is less than a communication range of the auxiliary wireless communication unit.
14. The patient display device according to any one of embodiments 1-13, wherein the wireless communication unit comprises a first wireless transceiver for communication with the patient external device and a second wireless transceiver for communication with the server.
15. The patient display device according to embodiment 14, wherein the second wireless transceiver is configured to be disabled to enable wireless communication using the first wireless transceiver.
16. The patient display device according to any one of embodiments 1-15, wherein at least one of:
   the patient display device is configured to authenticate the patient external device if a distance between the patient display device and the patient external device is less than a predetermined threshold value, and
   the patient display device is configured to be authenticated by the patient external device if a distance between the patient display device and the patient external device is less than a predetermined threshold value.
17. The patient display device according to embodiment 16, wherein the patient display device is configured to allow the transfer of data between the patient display device and the patient external device on the basis of the authentication.
18. The patient display device according to any one of the preceding embodiments, wherein the patient display device is a wearable external device or a handset.
19. The patient display device according to any one of the preceding embodiments, wherein the second application is configured to receive data related to a parameter of the implanted medical device.
20. The patient display device according to embodiment 19, wherein the second application is configured to receive data related to a sensor value received from the implanted medical device.
21. The patient display device according to embodiment 19, wherein the second application is configured to receive data related to a parameter related to at least one of:
   a battery status,
   a temperature,
   a time, and
   an error.
22. The patient display device according to any one of the preceding embodiments, wherein the patient display device is configured to encrypt the user input.
23. The patient display device according to embodiment 22, wherein the display is configured to encrypt the user input for decryption by the implantable medical device.
24. The patient display device according to any one of the preceding embodiments, wherein the patient display device is configured to decrypt the control interface received from the patient external device, for displaying the control interface on the display.
25. The patient display device according to any one of the preceding embodiments, wherein at least one of the first and second application is configured to receive data from an auxiliary external device and present the received data to the user.
26. The patient display device according to embodiment 25, wherein at least one of the first and second application is configured to receive data from an auxiliary external device comprising a scale for determining the weight of the user.
27. The patient display device according to embodiment 26, wherein at least one of the first and second application is configured to receive data related to the weight of the user from an auxiliary external device comprising a scale.
28. The patient display device according to embodiment 27, wherein the patient display device is configured to:
   wirelessly transmit the data related to the weight of the user to the patient external device, or
   wirelessly transmit an instruction derived from the data related to the weight of the user, or
   wirelessly transmit an instruction derived from a combination of the data related to the weight of the user and the implant control input received from the user.
29. A communication system for enabling communication between a patient display device and an implantable medical device, when implanted, the communication system comprising:
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
   wherein the patient remote external device comprises a wireless communication unit configured for wireless transmission of control commands based on the implant control input to the implantable medical device and configured for wireless communication with the patient display device.
30. The communication system according to embodiment 29, wherein the patient display device comprises a first log-in function and a second log-in function, and wherein the first log-in function gives the user access to the first application and wherein the first and second log-in function in combination gives the user access to the second application.
31. The communication system according to any one of embodiments 29 and 30, wherein the second application is configured to receive data related to a parameter of the implanted medical device.
32. The communication system according to embodiment 31, wherein the second application is configured to receive data related to a sensor value received from the implanted medical device.
33. The communication system according to embodiment 31, wherein the second application is configured to receive data related to a parameter related to at least one of
   a battery status,
   a temperature,
   a time, or
   an error.
34. The communication system according to any one of embodiments 29-33, wherein the patient display device is configured to encrypt the user input.
35. The communication system according to embodiment 34, wherein the display is configured to encrypt the user input for decryption by the implantable medical device.
36. The communication system according to embodiment 35, wherein the patient remote external device is configured to act as a router, transferring the encrypted user input from the patient display device to the implantable medical device without decryption.
37. The communication system according to any one of embodiments 29-36, wherein the patient remote external device is configured to encrypt at least one of the control interface and the control commands.
38. The communication system according to any one of embodiments 29-37, wherein the patient remote external device is configured to encrypt the control interface and wherein the patient display device is configured to decrypt the encrypted control interface.
39. A computer program product configured to run in a patient display device comprising a wireless communication unit, a display for displaying the received implant control interface as a remote display portal, and an input device for receiving implant control input from a user, the computer program product comprising:
   a first application for communication with a server or DDI,
   a second application for communication with an patient remote external device for transmission of the implant control input via the remote display portal of the patient remote external device for the communication with an implantable medical device, wherein the second application is configured to be accessed through the first application,
   a first log-in function using at least one of a password, pin code, fingerprint, or face recognition, and
   a second log-in function within the first application, using a private key from the user to authenticate for a defined time period a second hardware key of the patient remote external device,
wherein
   the first log-in function gives the user access to the first application and the first and second log-in function in combination gives the user access to the second application.
40. The computer program product according to embodiment 39, wherein the second application is configured to receive data related to a parameter of the implanted medical device.
41. The computer program product according to embodiment 40, wherein the second application is configured to receive data related to a sensor value received from the implanted medical device.
42. The computer program product according to embodiment 40, wherein the second application is configured to receive data related to a parameter related to at least one of
   a battery status,
   a temperature,
   a time, or
   an error.
43. The communication system, patient display device or computer program product according to any one of the preceding embodiments, wherein the implantable medical device comprises at least one of:
   an external heart compression device,
   an apparatus assisting the pump function of a heart of the patient,
   an apparatus assisting the pump function comprising a turbine bump placed within a patient's blood vessel for assisting the pump function of the heart,
   an operable artificial heart valve,
   an operable artificial heart valve for increasing the blood flow to the coronary arteries.
   an implantable drug delivery device,
   an implantable drug delivery device for injecting directly into a blood vessel and change the position of the injection site, all from within the patient's body,
   an implantable drug delivery device for injecting potency enhancing drugs into an erectile tissue of the patient,
   a hydraulic, mechanic, and/or electric constriction implant,
   an operable volume filling device,
   an operable gastric band,
   an operable implant for stretching the stomach wall of the patient for creating satiety,
   an implant configured to sense the frequency of the patient ingesting food,
   an operable cosmetic implant,
   an operable cosmetic implant for adjust the shape and/or size in the breast region of a patient,
   an implant controlling medical device for the emptying of a urinary bladder,
   an implant hindering urinary leakage,
   an implant hindering anal incontinence,
   an implant controlling the emptying of fecal matter,
   an implant monitoring an aneurysm,
   an implant for hindering the expansion of an aneurysm,
   an implant lubricating a joint,
   an implant for affecting the blood flow to an erectile tissue of the patient,
   an implant for simulating the engorgement of an erectile tissue,
   an implant with a reservoir for holding bodily fluids,
   an implant storing and/or emptying a bodily reservoir or a surgically created reservoir,
   an implant communicating with a database outside the body,
   an implant able to be programmed from outside the body,
   an implant able to be programmed from outside the body with a wireless signal,
   an implant treating impotence,
   an implant controlling the flow of eggs in the uterine tube,
   an implant controlling the flow of sperms in the uterine tube,
   an implant controlling the flow of sperms in the vas deferens,
   an implant for hindering the transportation of the sperm in the vas deferens,
   an implant treating osteoarthritis,
   an implant performing a test of parameters inside the body,
   an implant controlling specific treatment parameters from inside the body,
   an implant controlling bodily parameters from inside the body,
   an implant controlling the blood pressure,
   an implant controlling the blood pressure by affecting the dilatation of the renal artery,
   an implant controlling a drug treatment parameter,
   an implant controlling a parameter in the blood,
   an implant for adjusting or replacing any bone part of a body of the patient,
   an implant replacing an organ of the patient or part of an organ of the patient or the function thereof,
   a vascular treatment device,
   an implant adapted to move fluid inside the body of the patient,
   an implant configured to sense a parameter related to the patient swallowing,
   an implant configured to exercise a muscle with electrical or mechanical stimulation,
   an implant configured for emptying an intestine portion on command,
   an operable implant configured to be invaginated in the stomach of the patient to reduce the volume of the stomach substantially more than the volume of the device,
   an implant configured for emptying the urinary bladder from within the patient's body by compressing the bladder,
   an implant configured for draining fluid from within the patient's body,
   an implant configured for the active lubrication of a joint with an added lubrication fluid,
   an implant configured for removing clots and particles from the patient's blood stream,
   an implant configured for elongating or straightening a bone in the patient, to reduce scoliosis,
   a device to stimulate the brain for a several position to a focused point,
   an artificial stomach replacing the function of the natural stomach,
   an implant configured for adjusting the position of a female's urinary tract or bladder neck,
   an implant configured for stimulating the ampulla vas deference and creating temporary constriction.
44. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
45. The system according to anyone of the preceding embodiments, wherein the patient remote external device and the patient EID external device are an integrated unit.
46. The system according to anyone of the preceding embodiments, wherein the HCP dedicated device and the HCP EID external device are an integrated unit.
47. The system according to anyone of the preceding embodiments, comprising a measurement device or sensor adapted to deliver a measurement to at least one of the DDI, patent EID external device and a patient display device.
48. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.
49. A patient display device for communication with a patient external device for communication with the implantable medical device of any of embodiments 1-28 of aspect 386SE, wherein the patient display device comprises a first log-in function and a second log-in function, and wherein the first log-in function gives the user access to the first application and wherein the first and second log-in function in combination gives the user access to the second application.
50 A patient display device for communication with a patient external device for communication with the implantable medical device of any of embodiments 1-28 of aspect 386SE, wherein the first log-in function is configured to use at least one of a password, pin code, fingerprint, voice and face recognition, and
a second log-in function within the first application is configured to use a private key from the user to authenticate, for a defined time period, a second hardware key of the patient external device.
51. A patient display device for communication with a patient external device for communication with an implantable medical device, when implanted, the patient display device comprising:
a wireless communication unit,
a display, and
an input device for receiving implant control input from the user, wherein the patient display device is configured to:
run a first application for wireless communication with a server and/or DDI, and
run a second application for wireless communication with the patient external device for transmission of the implant control input to a remote display portal of the patient external device for the communication with the implantable medical device, wherein the second application is configured to be accessed through the first application.
52. A patient display device for communication with a patient external device for communication with the implantable medical device of any of embodiments 1-28 of aspect 386SE, wherein the patient display device comprises a first log-in function and a second log-in function, and wherein the first log-in function gives the user access to the first application and wherein the first and second log-in function in combination gives the user access to the second application.

### Aspect 386B—eHealth_General_General_App_in_App, Embodiments 1-43

1. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical
   device and configured for wireless communication with the patient display device, wherein
the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A communication system for enabling communication between a patient display device and an implantable medical device, when implanted, the communication system comprising:
   an implantable medical device,
   a patient display device, a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm, a control unit adapted to control pressure adjustments of the implantable member.
13. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device, wherein
the implantable medical device comprises a system for treating a female patient to avoid or promote pregnancy comprising a restriction device adapted to postoperatively restrict and release an oviduct of the patient.
18. A communication system for enabling communication between a patient display device and an implantable medical device, when implanted, the communication system comprising:
   an implantable medical device,
   a patient display device, a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A communication system for enabling communication between a patient display device and an implantable medical device, when implanted, the communication system comprising:
   an implantable medical device,
   a patient display device, a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The communication system according to any one of embodiments 1-21 or 31-35, wherein the patient display device comprises a first log-in function and a second log-in function, and wherein the first log-in function gives the user access to the first application and wherein the first and second log-in function in combination gives the user access to the second application.
23. The communication system according to any one of embodiments 1-21 or 31-35, wherein the second application is configured to receive data related to a parameter of the implanted medical device.
24. The communication system according to embodiment 23, wherein the second application is configured to receive data related to a sensor value received from the implanted medical device.
25. The communication system according to embodiment 23, wherein the second application is configured to receive data related to a parameter related to at least one of:
   a battery status,
   a temperature,
   a time, or
   an error.
26. The communication system according to any one of embodiments any one of embodiments 1-25 or 31-35, wherein the patient display device is configured to encrypt the user input.
27. The communication system according to embodiment 26, wherein the display is configured to encrypt the user input for decryption by the implantable medical device.
28. The communication system according to embodiment 27, wherein the patient remote external device is configured to act as a router, transferring the encrypted user input from the patient display device to the implantable medical device without decryption.
29. The communication system according to any one of embodiments 1-28 or 31-35, wherein the patient remote external device is configured to encrypt at least one of the control interface and the control commands.
30. The communication system according to any one of embodiments 1-29 or 31-35, wherein the patient remote external device is configured to encrypt the control interface and wherein the patient display device is configured to decrypt the encrypted control interface.
31. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an apparatus for treating obesity of an patient having a stomach with a food cavity, the apparatus comprising:
      a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
      at least one adjustable stretching device adapted to be at least substantially invaginated by a stomach wall portion of the patient with the outer surface of the stretching device resting against the stomach wall and adapted to stretch a portion of stomach wall, and
      a fluid connection device interconnecting the volume filling device and the stretching device.
32. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a device for treating obesity of a patient, the device comprising
      at least one operable stretching device (10; 110) implantable in the patient and adapted to stretch a portion of the patient's stomach wall (12), and
      an implantable control unit (42) for automatically controlling the operable stretching device, when the control unit and stretching device are implanted, to stretch the stomach wall portion in connection with the patient eating such that satiety is created,
      wherein the stretching device comprises a first and a second engaging part (10″; 110a; 110b; 210a; 210b), wherein:
      the first part is adapted to be engaged to a first area of the stomach wall, and
      the second part is adapted to be engaged to a second area of the stomach wall, and wherein
      the stretching device is adapted to stretch a portion of the stomach wall between the first area and the second area.
33. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises an obesity treatment device comprising:
      at least one operable stretching device (10; 10″; 110a; 110b; 410b; 50; 810) implantable in a patient and adapted to stretch a portion of the patient's stomach wall (12) and
      an implantable operation device (90; 42; 54; 40; 217; 218; 452) for operating the stretching device, when implanted, to stretch the stomach wall portion such that satiety is created,
      wherein the operable stretching device is adapted to be placed against the outside of the stomach wall.
34. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a device for treating obesity of a patient, the device comprising:
      at least one operable stretching device implantable in the patient and adapted to stretch a portion of the patient's stomach wall, and
      an implantable control unit for controlling the operable stretching device, when the control unit and the stretching device are implanted, to stretch the stomach wall portion in connection with the patient eating such that satiety is created,
      wherein the stretching device comprises a first engaging part and a second engaging part, wherein:
      the first engaging part is adapted to be engaged to a first area of the stomach wall, and
      the second engaging part is adapted to be engaged to a second area of the stomach wall, and wherein
      the stretching device is adapted to stretch a portion of the stomach wall between the first area and the second area.
35. A communication system for enabling communication between a patient display device and an implantable medical device, when implanted, the communication system comprising:
   an implantable medical device,
   a patient display device,
   a server or DDI, and
   a patient remote external device, wherein:
   the patient display device comprises:
   a wireless communication unit configured for wirelessly receiving an implant control interface as a remote display portal from the patient remote external device, the wireless communication unit further being configured for wirelessly transmitting implant control user input to the patient remote external device,
   a display for displaying the received implant control interface as a remote display portal, and
   an input device for receiving implant control input from the user,
   wherein the patient display device is configured:
   to run a first application for wireless communication with the server, and
   to run a second application for wireless communication with the patient remote external device for transmission of the implant control input to the remote display portal of the patient remote external device for the communication with the implantable medical device, and
wherein the patient remote external device comprises a wireless communication unit configured for wireless transmission of control commands based on the implant control input to the implantable medical device and configured for wireless communication with the patient display device, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 387SE—eHealth_General_Encryption_End-to-End, Embodiments 1-28

1. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption.
2. The communication system according to embodiment 1, wherein the patient display device is configured to wirelessly receive an implant control interface from the patient external device to be displayed on the display.
3. The communication system according to any one of embodiments 1-2, wherein at least two of:
   the wireless communication unit of the server,
   the wireless communication unit of the patient display device,
   the wireless communication unit of the patient external device, and
   the wireless communication unit of the implantable medical device,
   is configured for wireless communication using a standard network protocol.
4. The communication system according to any one of embodiments 1-2, wherein at least two of:
   the wireless communication unit of the server,
   the wireless communication unit of the patient display device,
   the wireless communication unit of the patient external device, and
   the wireless communication unit of the implantable medical device,
   is configured for wireless communication using a proprietary network protocol.
5. The communication system according to any one of embodiments 1-4, wherein the wireless communication unit of the patient external device is configured to:
   use a first network protocol for communication with the implantable medical device and use a second network protocol for communication with the server, or
   use a first network protocol for communication with the implantable medical device and use a second network protocol for communication with the patient display device.
6. The communication system according to any one of embodiments 1-5, wherein the wireless communication unit of the patient external device is configured to:
   use a first frequency band for communication with the implantable medical device and use a second frequency band for communication with the server, or
   use a first frequency band for communication with the implantable medical device and use a second frequency band for communication with the patient display device.
7. The communication system according to any one of embodiments 1-6, wherein the wireless communication unit of the patient display device is configured to use a first network protocol for communication with the patient external device and use a second network protocol for communication with the server.
8. The communication system according to any one of embodiments 1-7, wherein the wireless communication unit of the patient display device is configured to use a first frequency band for communication with the patient external device and use a second frequency band for communication with the server.
9. The communication system according to any one of embodiments 1-8, wherein the wireless communication unit of the server is configured to use a first network protocol for communication with the patient external device and use a second network protocol for communication with the patient display device.
10. The communication system according to any one of embodiments 1-9, wherein the wireless communication unit of the server is configured to use a first frequency band for communication with the patient external device and use a second frequency band for communication with the patient display device.
11. The communication system according to any one of embodiments 1-10, wherein the wireless communication unit of at least one of the server, the patient display device, the patient external device, and the implantable medical device comprises a Bluetooth transceiver.
12. The communication system according to any one of embodiments 1-11, wherein the wireless communication unit of at least one of the server, the patient display device, the patient external device, and the implantable medical device comprises a UWB transceiver.
13. The communication system according to embodiment 3, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
14. The communication system according to any one of the preceding embodiments, wherein the wireless communication unit of the patient external device comprises a first wireless transceiver for wireless communication with the implantable medical device, and a second wireless transceiver for wireless communication with the server, and wherein the second wireless transceiver has a longer effective range than the first wireless transceiver.
15. The communication system according to any one of the preceding embodiments, wherein the wireless communication unit of the patient external device comprises a first wireless transceiver for wireless communication with the implantable medical device, and a second wireless transceiver for wireless communication with the patient display device, and wherein the second wireless transceiver has a longer effective range than the first wireless transceiver.
16. The communication system according to any one of the preceding embodiments, wherein the wireless communication unit of the patient display device comprises a first wireless transceiver for wireless communication with the patient external device, and a second wireless transceiver for wireless communication with the server, and wherein the second wireless transceiver has a longer effective range than the first wireless transceiver.
17. The communication system according to any one of embodiment 14-17, wherein the second wireless transceiver has an effective range being one of 2 times, 4 times, 8 times 20 times, 50 times or 100 times longer than the first wireless transceiver.
18. The communication system according to any one of embodiments 14-17, wherein the second wireless transceiver is configured to be disabled to enable wireless communication using the first wireless transceiver.
19. The communication system according to any one of the preceding embodiments, wherein at least one of
   the patient display device is configured to authenticate the patient external device if a distance between the patient display device and the patient external device is less than a predetermined threshold value,
   the patient display device is configured to be authenticated by the patient external device if a distance between the patient display device and the patient external device is less than a predetermined threshold value,
   the patient display device is configured to authenticate the implantable medical device if a distance between the patient display device and the implantable medical device is less than a predetermined threshold value,
   the patient display device is configured to be authenticated by the implantable medical device if a distance between the patient display device and the implantable medical device is less than a predetermined threshold value,
   the patient external device is configured to authenticate the patient display device if a distance between the patient external device and the patient display device is less than a predetermined threshold value,
   the patient external device is configured to be authenticated by the patient display device if a distance between the patient external device and the patient display device is less than a predetermined threshold value,
   the patient external device is configured to authenticate the implantable medical device if a distance between the patient external device and the implantable medical device is less than a predetermined threshold value, and
   the patient external device is configured to be authenticated by the implantable medical device if a distance between the patient external device and the implantable medical device is less than a predetermined threshold value.
20. The communication system according to embodiment 19, wherein the patient display device is configured to allow the transfer of data between the patient display device and the patient external device on the basis of the authentication.
21. The communication system according to embodiment 19, wherein the patient external device is configured to allow the transfer of data between the patient display device and the patient external device on the basis of the authentication.
22. The communication system according to embodiment 19, wherein the patient external device is configured to allow the transfer of data between the patient external device and the implantable medical device on the basis of the authentication.
23. The communication system according to any one of the preceding embodiments, wherein the patient display device is a wearable patient external device or a handset.
24. The communication system according to any one of the preceding embodiments, wherein the data encrypted by the implantable medical device is related to at least one of
   a battery status,
   a temperature,
   a time, or
   an error.
25. A server for use in the communication system according to any one of embodiments 1-24.
26. A patient display device for use in the communication system according to any one of embodiments 1-24.
27. A patient external device for use in the communication system according to any one of embodiments 1-24.
28. An implantable medical device for use in the communication system according to any one of embodiments 1-24.
29. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
30. The system according to anyone of the preceding embodiments, wherein the patient remote external device and the patient EID external device are an integrated unit.
31. The system according to anyone of the preceding embodiments, wherein the HCP dedicated device and the HCP EID external device are an integrated unit.
32. The system according to anyone of the preceding embodiments, comprising a measurement device or sensor adapted to deliver a measurement to at least one of the DDI, patent EID external device and a patient display device.
33. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.

### Aspect 387B—eHealth_General_Encryption_End-to-End, Embodiments 1-49

1. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with
at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with
at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with
at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A communication system for enabling communication between a patient display device, a patient external device, a server and an implantable medical device, the communication system comprising: a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable lubrication device comprising: a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein the implantable medical device, wherein
   the implantable medical device comprises a system for treating a female patient to avoid or promote pregnancy comprising a restriction device adapted to postoperatively restrict and release an oviduct of the patient.
18. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The communication system according to any one of embodiments 1-22 or 50-54, wherein the patient display device is configured to wirelessly receive an implant control interface from the patient external device to be displayed on the display.
23. The communication system according to any one of embodiments 1-23 or 50-54, wherein at least two of
   the wireless communication unit of the server,
   the wireless communication unit of the patient display device,
   the wireless communication unit of the patient external device, and
   the wireless communication unit of the implantable medical device,
   is configured for wireless communication using a standard network protocol.
24. The communication system according to any one of embodiments 1-22 or 50-54, wherein at least two of
   the wireless communication unit of the server,
   the wireless communication unit of the patient display device,
   the wireless communication unit of the patient external device, and
   the wireless communication unit of the implantable medical device,
   is configured for wireless communication using a proprietary network protocol.
25. The communication system according to any one of embodiments 1-24 or 50-54, wherein the wireless communication unit of the patient external device is configured to:
   use a first network protocol for communication with the implantable medical device and use a second network protocol for communication with the server, or
   use a first network protocol for communication with the implantable medical device and use a second network protocol for communication with the patient display device.
26. The communication system according to any one of embodiments 1-25 or 50-54, wherein the wireless communication unit of the patient external device is configured to:
   use a first frequency band for communication with the implantable medical device and use a second frequency band for communication with the server, or
   use a first frequency band for communication with the implantable medical device and use a second frequency band for communication with the patient display device.
27. The communication system according to any one of embodiments 1-26 or 50-54, wherein the wireless communication unit of the patient display device is configured to use a first network protocol for communication with the patient external device and use a second network protocol for communication with the server.
28. The communication system according to any one of embodiments 1-27 or 50-54, wherein the wireless communication unit of the patient display device is configured to use a first frequency band for communication with the patient external device and use a second frequency band for communication with the server.
29. The communication system according to any one of embodiments 1-28 or 50-54, wherein the wireless communication unit of the server is configured to use a first network protocol for communication with the patient external device and use a second network protocol for communication with the patient display device.
30. The communication system according to any one of embodiments 1-29 or 50-54, wherein the wireless communication unit of the server is configured to use a first frequency band for communication with the patient external device and use a second frequency band for communication with the patient display device.
31. The communication system according to any one of embodiments 1-30 or 50-54, wherein the wireless communication unit of at least one of the server, the patient display device, the patient external device, and the implantable medical device comprises a Bluetooth transceiver.
32. The communication system according to any one of embodiments 1-31 or 50-54, wherein the wireless communication unit of at least one of the server, the patient display device, the patient external device, and the implantable medical device comprises a UWB transceiver.
33. The communication system according to embodiment 23, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
34. The communication system according to any one of the preceding embodiments or 50-54, wherein the wireless communication unit of the patient external device comprises a first wireless transceiver for wireless communication with the implantable medical device, and a second wireless transceiver for wireless communication with the server, and wherein the second wireless transceiver has a longer effective range than the first wireless transceiver.
36. The communication system according to any one of the preceding embodiments or 50-54, wherein the wireless communication unit of the patient external device comprises a first wireless transceiver for wireless communication with the implantable medical device, and a second wireless transceiver for wireless communication with the patient display device, and wherein the second wireless transceiver has a longer effective range than the first wireless transceiver.
37. The communication system according to any one of the preceding embodiments or 50-54, wherein the wireless communication unit of the patient display device comprises a first wireless transceiver for wireless communication with the patient external device, and a second wireless transceiver for wireless communication with the server, and wherein the second wireless transceiver has a longer effective range than the first wireless transceiver.
38. The communication system according to any one of embodiment 34-36, wherein the second wireless transceiver has an effective range being one of: 2 times, 4 times, 8 times 20 times, 50 times or 100 times longer than the first wireless transceiver.
39. The communication system according to any one of embodiments 34-36, wherein the second wireless transceiver is configured to be disabled to enable wireless communication using the first wireless transceiver.
40. The communication system according to any one of the preceding embodiments or 50-54, wherein at least one of:
   the patient display device is configured to authenticate the patient external device if a distance between the patient display device and the patient external device is less than a predetermined threshold value,
   the patient display device is configured to be authenticated by the patient external device if a distance between the patient display device and the patient external device is less than a predetermined threshold value,
   the patient display device is configured to authenticate the implantable medical device if a distance between the patient display device and the implantable medical device is less than a predetermined threshold value,
   the patient display device is configured to be authenticated by the implantable medical device if a distance between the patient display device and the implantable medical device is less than a predetermined threshold value,
   the patient external device is configured to authenticate the patient display device if a distance between the patient external device and the patient display device is less than a predetermined threshold value,
   the patient external device is configured to be authenticated by the patient display device if a distance between the patient external device and the patient display device is less than a predetermined threshold value,
   the patient external device is configured to authenticate the implantable medical device if a distance between the patient external device and the implantable medical device is less than a predetermined threshold value, and
   the patient external device is configured to be authenticated by the implantable medical device if a distance between the patient external device and the implantable medical device is less than a predetermined threshold value.
41. The communication system according to embodiment 40, wherein the patient display device is configured to allow the transfer of data between the patient display device and the patient external device on the basis of the authentication.
42. The communication system according to embodiment 40, wherein the patient external device is configured to allow the transfer of data between the patient display device and the patient external device on the basis of the authentication.
43. The communication system according to embodiment 40, wherein the patient external device is configured to allow the transfer of data between the patient external device and the implantable medical device on the basis of the authentication.
44. The communication system according to any one of the preceding embodiments or 50-54,
wherein the patient display device is a wearable patient external device or a handset.
45. The communication system according to any one of the preceding embodiments or 50-54, wherein the data encrypted by the implantable medical device is related to at least one of:
   a battery status,
   a temperature,
   a time, or
   an error.
46. A server for use in the communication system according to any one of embodiments 1-45 or 50-54.
47. A patient display device for use in the communication system according to any one of embodiments 1-45 or 50-54.
48. A patient external device for use in the communication system according to any one of embodiments 1-45 or 50-54.
49. An implantable medical device for use in the communication system according to any one of embodiments 1-45 or 50-54.
50. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
51. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
the implantable medical device comprises a device for treating obesity of a patient, the device comprising
   at least one operable stretching device (10; 110) implantable in the patient and adapted to stretch a portion of the patient's stomach wall (12), and an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
52. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
53. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
54. A communication system for enabling communication between a patient display device, a patient external device, a server and an implantable medical device, the communication system comprising:
a server,
a patient external device, and
an implantable medical device,
wherein a patient display device is adapted to co-operate with at least one of the patient external device and the server and further adapted to use:
a wireless communication unit for wirelessly communicating with at least one of the patient external device and the server,
a display, and
an input device for receiving input from the user,
wherein the patient external device comprises a wireless communication unit configured for wireless transmission of control commands to the implantable medical device and configured for wireless communication with at least one of the patient display device and the server,
wherein the server comprises a wireless communication unit configured for wireless communication with at least one of the patient display device and the patient external device,
wherein the implantable medical device comprises a wireless communication unit configured for wireless communication with the patient external device,
wherein the implantable medical device comprises an encryption unit and is configured to: encrypt data destined for the server, transmit the data to the server via at least one of the patient external device and a second external device or patient EID, wherein at least one of the patient external device and the patient EID,
wherein the implantable medical device comprises an encryption unit and is configured to: encrypt data destined for the patient display device, transmit the data to the patient display device via the patient external device, wherein the patient external device acts as a router transferring the data without full decryption, or
wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
wherein the patient display device comprises an encryption unit and is configured to: encrypt data destined for the implantable medical device, transmit the data to the implantable medical device via the patient external device, wherein the patient external device acts as a router transferring the data with or without full decryption, or
wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption wherein
the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 387C—eHealth_General_Encryption_End-to-End

1. A communication system for communication of data to or from an implantable medical device, the communication system comprising:
an implantable medical device,
a server,
a patient external device comprising:
a first wireless communication unit configured for wireless transmission of control commands or data to or from the implantable medical device, and
a second wireless communication unit for wireless communication with the server,
a patient display device adapted to communicate with at least one of the patient external device and the server,
wherein the implantable medical device comprises an encryption unit and is configured to: encrypt data destined for the server,
transmit the data to the server via at least one of the patient external device.
2. A communication system for enabling communication to or from an implantable medical device, the communication system comprising:
a server,
a patient external device comprising:
a first wireless communication unit configured for wireless transmission of control commands or data to or from the implantable medical device, and
a second wireless communication unit for wireless communication with the server,
an implantable medical device,
a patient display device adapted to communicate with at least one of the patient external device and the server,
wherein the implantable medical device comprises an encryption unit and is configured to: encrypt data destined for the patient display device,
transmit the data to the patient display device via the patient external device,
wherein the patient external device acts as a router transferring the data without full decryption.
3. A communication system for enabling communication to or from an implantable medical implant, the communication system comprising:
a server,
a patient external device comprising:
a first wireless communication unit configured for wireless transmission of control commands or data to or from the implantable medical device, and
a second wireless communication unit for wireless communication with the server,
an implantable medical device,
a patient display device adapted to communicate with at least one of the patient external device and the server,
wherein the server comprises an encryption unit and is configured to: encrypt data destined for the implantable medical device,
transmit the data to the implantable medical device via the patient external device,
wherein the patient external device acts as a router transferring the data without full decryption.
4. A communication system for enabling communication to or from a medical implant, the communication system comprising:
a server,
a patient external device comprising:
a first wireless communication unit configured for wireless transmission of control commands or data to or from the implantable medical device, and
a second wireless communication unit for wireless communication with the server,
an implantable medical device,
a patient display device adapted to communicate with at least one of the patient external device and the server,
wherein the patient display device comprises an encryption unit and is configured to: encrypt data destined for the implantable medical device,
transmit the data to the implantable medical device via the patient external device, wherein the patient external device acts as a router transferring the data with or without full decryption.
5. A communication system for enabling communication to or from a medical implant, the communication system comprising:
a server,
a patient external device comprising:
a first wireless communication unit configured for wireless transmission of control commands or data to or from the implantable medical device, and
a second wireless communication unit for wireless communication with the server,
an implantable medical device,
a patient display device adapted to communicate with at least one of the patient external device and the server,
wherein the patient display device comprises an encryption unit and is configured to: encrypt data destined for the implantable medical device,
transmit the data to the implantable medical device via the server and/or the patient external device,
wherein the server and/or the patient external device acts as a router transferring the data with or without full decryption.
6. The communication system according to any one of the preceding embodiments, further comprising: a display connected to the patient display device, and
an input device for receiving input from the user, the input device being connected to the patient display device,
wherein the patient display device is configured to receive an implant control interface from the patient external device to be displayed on the display, and wherein the patient display device is configured to transmit any control input received form the input device to the patient external device.
7. The communication system according to any one of the preceding embodiments, wherein at least two of:
the server,
the patient display device,
the patient external device, and
the implantable medical device,
are configured for wireless communication using a standard network protocol.
8. The communication system according to any one of the preceding embodiments, wherein at least two of:
the server,
the patient display device,
the patient external device, and
the implantable medical device,
are configured for wireless communication using a proprietary network protocol.
9. The communication system according to any one of the preceding embodiments, wherein the patient external device is configured to:
use a first network protocol for communication with the implantable medical device and use a second network protocol for communication with the server, or
use a first network protocol for communication with the implantable medical device and use a second network protocol for communication with the patient display device.
6. The communication system according to any one of the preceding embodiments, wherein the patient external device is configured to:
use a first frequency band for communication with the implantable medical device and use a second frequency band for communication with the server, or
use a first frequency band for communication with the implantable medical device and use a second frequency band for communication with the patient display device.
10. The communication system according to any one of the preceding embodiments, wherein the patient display device is configured to use a first network protocol for communication with the patient external device and use a second network protocol for communication with the server.
11. The communication system according to any one of the preceding embodiments, wherein the patient display device is configured to use a first frequency band for communication with the patient external device and use a second frequency band for communication with the server.
12. The communication system according to any one of the preceding embodiments, wherein at least one of the patient display device, the patient external device, and the implantable medical device comprises a Bluetooth transceiver.
13. The communication system according to any one of the preceding embodiments, wherein at least one the patient display device, the patient external device, and the implantable medical device comprises a UWB transceiver.
14. The communication system according to embodiment 3, wherein the standard network protocol is one from the list of Radio Frequency type protocol, RFID type protocol, WLAN type protocol, Bluetooth type protocol, BLE type protocol, NFC type protocol, 3G/4G/5G type protocol, and GSM type protocol.
15. The communication system according to any one of the preceding embodiments, wherein the patient external device comprises a first wireless transceiver for wireless communication with the implantable medical device, and a second wireless transceiver for wireless communication with the server, and wherein the second wireless transceiver has a longer effective range than the first wireless transceiver.
16. The communication system according to any one of the preceding embodiments, wherein the patient external device comprises a first wireless transceiver for wireless communication with the implantable medical device, and a second wireless transceiver for wireless communication with the patient display device, and wherein the second wireless transceiver has a longer effective range than the first wireless transceiver.
17. The communication system according to any one of the preceding embodiments, wherein the patient display device comprises a first wireless transceiver for wireless communication with the patient external device, and a second wireless transceiver for wireless communication with the server, and wherein the second wireless transceiver has a longer effective range than the first wireless transceiver.
18. The communication system according to any one of embodiment 15-17, wherein the second wireless transceiver has an effective range being one of 2 times, 4 times, 8 times 20 times, 50 times or 100 times longer than the first wireless transceiver.
19. The communication system according to any one of embodiments 15-17, wherein the second wireless transceiver is configured to be disabled while the first wireless transceiver is enabled.
20. The communication system according to any one of the preceding embodiments, wherein the patient display device is configured to allow the transfer of data between the patient display device and the patient external device based on that a distance between the patient display device and the patient external device is less than a predetermined threshold value.
21. The communication system according to any one of the preceding embodiments, wherein the patient external device is configured to allow the transfer of data between the patient display device and the patient external device based on that a distance between the patient display device and the patient external device is less than a predetermined threshold value.
22. The communication system according to any one of the preceding embodiments, wherein the patient external device is configured to allow the transfer of data between the patient external device and the implantable medical device based on that a distance between the patient display device and the patient external device is less than a predetermined threshold value.
23. The communication system according to any one of the preceding embodiments, wherein the patient display device is a wearable patient display device or a handset.
24. The communication system according to any one of the preceding embodiments, wherein the implantable medical device is configured to transmit encrypted data related to at least one of a battery status, a temperature, a time, or an error.
25. A server for use in the communication system according to any one of embodiments 1-24.
26. A patient display device for use in the communication system according to any one of embodiments 1-24.
27. A patient external device for use in the communication system according to any one of embodiments 1-24.
28. An implantable medical device for use in the communication system according to any one of embodiments 1-24.
29. The communication system according to any one of the preceding embodiments, wherein at least one of:
The server comprises or is adapted to receive a health care provider private key for encrypting and/or decrypting data,
the patient display device comprises or is adapted to receive a patient display private key for encrypting and/or decrypting data,
the patient external device comprises or is adapted to receive a patient private key for encrypting and/or decrypting data, and
the implantable medical device comprises a medical device private key for encrypting or decrypting data.
30. The communication system according to embodiment 29, wherein at least one of the health care provider private key, patient display private key, the patient private key, the medical device private key comprises a hardware or a software key.

### Aspect 396SE—eHealth_General_Communication_Dual

1. A system configured for changing pre-programmed treatment settings of an implantable medical device, when implanted in a patient, from a distant remote location in relation to the patient, the system comprising:
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function.
2. The system configured for changing pre-programmed treatment settings of an implantable medical device according to claim 1, wherein the patient providing a patient private key device adapted to be provided to the patient EID external device by the patient via at least one of: a reading slot or comparable for the patient private key device, a RFID communication or other close distance wireless activation communication or electrical direct contact,
wherein said patient EID external device comprises at least one of:
a reading slot or comparable for the HCP private key device,
a RFID communication, and
other close distance wireless activation communication or electrical direct contact.
3. The system configured for changing pre-programmed treatment settings of an implantable medical device according to claim 1-2, wherein the wherein said patient EID external device further comprises at least one wireless transceiver configured for communication with the implanted medical device through a second network protocol.
4. The system according to embodiment 1-3, wherein at least one of the patient private key device or HCP private key device comprises a hardware key.
5. The system according to any preceding embodiment, wherein the private key device is at least one of, a smartcard, a key-ring device, a watch an arm or wrist band a neckless or any shaped device.
6. The system according to any of the preceding embodiments, wherein at least two of:
   the HCP EID external device,
   the patient EID external device,
   the HCP private key device,
   the patient private key device, and
   the DDI is configured for wireless communication using a standard network protocol.
7. The system according to any of the preceding embodiments, wherein at least two of:
   the HCP EID external device,
   the patient EID external device,
   the HCP private key device,
   the patient private key device, and
   the DDI is configured for wireless communication using a proprietary network protocol.
8. The system according to any of the preceding embodiments, wherein the patient EID external device is configured to use a first network protocol for communication with the implantable medical device and use a second network protocol for communication with the DDI.
9. The system according to any of the preceding embodiments, wherein the patient EID external device is configured to use a first frequency band for communication with the implantable medical device and use a second frequency band for communication with the DDI.
10. The system according to any of the preceding embodiments, wherein the DDI is configured to
   use a first frequency band for communication with the patient EID external device and a second frequency band for communication with the patient private key device.
11. The system according to any of the preceding embodiments, wherein at least one of the HCP EID external device, the patient EID external device, the HCP private key device, the patient private key device and the DDI comprises a Bluetooth transceiver.
12. The system according to any of the preceding embodiments, wherein at least one of the HCP EID external device, the patient EID external device, the HCP private key device, the patient private key device and the DDI comprises a UWB transceiver.
13. The system according to embodiment 4, wherein the standard network protocol is one from the list of
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
14. The system according to any of the preceding embodiments, wherein the patient EID external device comprises a first wireless transceiver for wireless communication with the implantable medical device, and a second wireless transceiver for wireless communication with the DDI, and wherein the second wireless transceiver has longer effective range than the first wireless transceiver.
15. The system according to any of the preceding embodiments, wherein the patient private key device comprises a first wireless transceiver for wireless communication with the HCP EID external device, and a second wireless transceiver for wireless communication with the DDI, and wherein the second wireless transceiver has longer effective range than the first wireless transceiver.
16. The system according to embodiments 12 or 13, wherein the second wireless transceiver has an effective range being one of 2 times, 4 times, 8 time, 20 times, 50 times or 100 times longer than the effective range of the first wireless transceiver.
17. The system according to any of embodiments 12-14, wherein the second wireless transceiver is configured to be disabled to enable wireless communication using the first wireless transceiver.
18. The system according to any of the preceding embodiments, wherein the patient EID external device is configured to allow transfer of data between the EID external device and the implantable medical device on the basis of an authentication of the patient EID external device.
19. The system according to any of the preceding embodiments, wherein the patient EID external device is a wearable patient external device or a handset.
20. The system according to any of the preceding embodiments, wherein the data encrypted by the implantable medical device is related to at least one of
   a battery status,
   a temperature,
   a time, or
   an error.
21. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
22. The system according to anyone of the preceding embodiments, wherein the patient remote external device and the patient EID external device are an integrated unit.
23. The system according to anyone of the preceding embodiments, wherein the HCP dedicated device and the HCP EID external device are an integrated unit.
24. The system according to anyone of the preceding embodiments, comprising a measurement device or sensor adapted to deliver a measurement to at least one of the DDI, patent EID external device and a patient display device.
25. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.
26. A system configured for changing pre-programmed treatment settings of an implantable medical device according to claim 1-25, wherein
the HCP, HCP EID, or HCP via HCP EID are adapted to be providing changed pre-programmed settings of an implant of a human or mammal to:
a communication unit configured to receive such changed pre-programmed settings in an authenticated file, wherein the communication unit is controlled by a first chip, a first circuit, or a first chip and a first circuit, adapted to further provide the authenticated file to:
an decryption unit for decrypting the file received, controlled by a second chip, a second circuit, or a second chip and a second circuit adapted to not be directly in contact with any external device, adapted to be received via:
a narrow secure communication tunnel between the first chip, a first circuit, or a first chip and a first circuit, and the second chip, a second circuit, or a second chip and a second circuit, wherein the communication unit is adapted to only communicate with the decryption unit via the narrow secure communication tunnel, and wherein the decryption unit is adapted to authenticate the HCP provider of the authenticated file and that the data of the file has not been tampered with.
27. A system according to claim 1-26, wherein the authenticated file is adapted to be received indirect by the decryption unit adapted to decrypt the received data and use the decrypted data after the decryption unit has authenticated the HCP, HCP EID, or HCP and HCP EID as originator of the received data of the authenticated file and that the file or data has not been tampered with.
28. A system according to claim 1-27, wherein the authenticated file comprises instruction about selected programmed settings of steps of action, wherein use of the decrypted data of the authenticated file comprises instructions to select anyone of the pre-programmed settings of steps of actions of the implant.
29. A system according to claim 1-28, wherein the implant is adapted to authenticate, decrypt or authenticate and decrypt the received data using a first key.
30. A system according to claim 1-29, wherein the HCP private key, patient private key, or HCP private key and patient private key comprising a hardware key.
31. A system according to claim 1-30, wherein the HCP private key, patient private key, or HCP private key and patient private key comprising an advanced software key.
32. A system according to claim 1-31, wherein authenticated file is adapted to be received by a storage unit of the decryption unit comprising at least one of authenticating information and key related information, related to the HCP, HCP EID or HCP and HCP EID, wherein the decryption unit is adapted to compare stored information with received authenticated file data related to at least one of authentication and decryption.

### Aspect 396B—eHealth_General_Communication_Dual

1. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of: a reading slot or comparable for the HCP private key device, a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact; wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact; wherein said HCP EID external device further comprises at least one wireless transceiver configured for communication with a data infrastructure server, DDI, through a first network protocol, wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol, wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
a reading slot or comparable for the HCP private key device,
a RFID communication, and other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of: a reading slot or comparable for the HCP private key device, a RFID communication, and other close distance wireless activation communication or electrical direct contact;
wherein said HCP EID external device further comprises at least one wireless transceiver configured for communication with a data infrastructure server, DDI, through a first network protocol,
wherein said system further comprises: a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device, wherein
the implantable medical device comprises a system for treating a female patient to avoid or promote pregnancy comprising a restriction device adapted to postoperatively restrict and release an oviduct of the patient.
18. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact; wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other
close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device, a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system configured for changing pre-programmed treatment settings of an implantable medical device according to any one of embodiments 1-21 or 53-57, wherein the patient providing a patient private key device adapted to be provided to the patient EID external device by the patient via at least one of a reading slot or comparable for the patient private key device, a RFID communication or other close distance wireless activation communication or electrical direct contact,
wherein said patient EID external device comprises at least one of:
a reading slot or comparable for the HCP private key device,
a RFID communication, and
other close distance wireless activation communication or electrical direct contact.
23. The system configured for changing pre-programmed treatment settings of an implantable medical device according to any one of embodiments 1-21 or 53-57, wherein the wherein said patient EID external device further comprises at least one wireless transceiver configured for communication with the implanted medical device through a second network protocol.
24. The system configured for changing pre-programmed treatment settings of an implantable medical device according to any one of embodiments 1-23 or 53-57, wherein at least one of the patient private key device or HCP private key device comprises a hardware key.
25. The system configured for changing pre-programmed treatment settings of an implantable medical device according to any one of embodiments 1-24 or 53-57, wherein the private key device is at least one of, a smartcard, a key-ring device, a watch an arm or wrist band a neckless or any shaped device.
26. The system configured for changing pre-programmed treatment settings of an implantable medical device according to any one of embodiments 1-25 or 53-57, wherein at least two of:
   the HCP EID external device,
   the patient EID external device,
   the HCP private key device,
   the patient private key device, and
   the DDI is configured for wireless communication using a standard network protocol.
27. The system configured for changing pre-programmed treatment settings of an implantable medical device according to any one of embodiments 1-26 or 53-57, wherein at least two of:
   the HCP EID external device,
   the patient EID external device,
   the HCP private key device,
   the patient private key device, and
   the DDI is configured for wireless communication using a proprietary network protocol.
28. The system configured for changing pre-programmed treatment settings of an implantable medical device according to any one of embodiments 1-27 or 53-57, wherein the patient EID external device is configured to use a first network protocol for communication with the implantable medical device and use a second network protocol for communication with the DDI.
29. The system configured for changing pre-programmed treatment settings of an implantable medical device according to any one of embodiments 1-28 or 53-57, wherein the patient EID external device is configured to use a first frequency band for communication with the implantable medical device and use a second frequency band for communication with the DDI.
30. The system configured for changing pre-programmed treatment settings of an implantable medical device according to any one of embodiments 1-29 or 53-57, wherein the DDI is configured to use a first frequency band for communication with the patient EID external device and a second frequency band for communication with the patient private key device.
31. The system configured for changing pre-programmed treatment settings of an implantable medical device according to any one of embodiments 1-30 or 53-57, wherein at least one of the HCP EID external device, the patient EID external device, the HCP private key device, the patient private key device and the DDI comprises a Bluetooth transceiver.
32. The system configured for changing pre-programmed treatment settings of an implantable medical device according to any one of embodiments 1-31 or 53-57, wherein at least one of the HCP EID external device, the patient EID external device, the HCP private key device, the patient private key device and the DDI comprises a UWB transceiver.
33. The system configured for changing pre-programmed treatment settings of an implantable medical device according to embodiment 24, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
34. The system configured for changing pre-programmed treatment settings of an implantable medical device according to any one of embodiments 1-33 or 53-57, wherein the patient EID external device comprises a first wireless transceiver for wireless communication with the implantable medical device, and a second wireless transceiver for wireless communication with the DDI, and wherein the second wireless transceiver has longer effective range than the first wireless transceiver.
35. The system configured for changing pre-programmed treatment settings of an implantable medical device according to any one of embodiments 1-34 or 53-57, wherein the patient private key device comprises a first wireless transceiver for wireless communication with the HCP EID external device, and a second wireless transceiver for wireless communication with the DDI, and wherein the second wireless transceiver has longer effective range than the first wireless transceiver.
36. The system configured for changing pre-programmed treatment settings of an implantable medical device according to any one of embodiments 32 or 33, wherein the second wireless transceiver has an effective range being one of: 2 times, 4 times, 8 time, 20 times, 50 times or 100 times longer than the effective range of the first wireless transceiver.
37. The system configured for changing pre-programmed treatment settings of an implantable medical device according to any one of embodiments 32-34, wherein the second wireless transceiver is configured to be disabled to enable wireless communication using the first wireless transceiver.
38. The system configured for changing pre-programmed treatment settings of an implantable medical device according to any one of embodiments 1-38 or 53-57, wherein the patient EID external device is configured to allow transfer of data between the EID external device and the implantable medical device on the basis of an authentication of the patient EID external device.
39. The system configured for changing pre-programmed treatment settings of an implantable medical device according to any one of embodiments 1-40 or 53-57, wherein the patient EID external device is a wearable patient external device or a handset.
40. The system configured for changing pre-programmed treatment settings of an implantable medical device according to any one of embodiments 1-39 or 53-57, wherein the data encrypted by the implantable medical device is related to at least one of
   a battery status,
   a temperature,
   a time, or
   an error.
41. The system configured for changing pre-programmed treatment settings of an implantable medical device according to any one of embodiments 1-40 or 53-57, comprising a master private key device configured to allow issuance of new private key device, wherein the HCP or HCP admin have such master private key device adapted to be able to replace and pair a new patient private key device or HCP private key device into the system, through the HCP EID external device.
42. The system configured for changing pre-programmed treatment settings of an implantable medical device according to any one of embodiments 1-42 or 53-57, wherein the patient remote external device and the patient EID external device are an integrated unit.
43. The system configured for changing pre-programmed treatment settings of an implantable medical device according to any one of embodiments 1-43 or 53-57, wherein the HCP dedicated device and the HCP EID external device are an integrated unit.
44. The system configured for changing pre-programmed treatment settings of an implantable medical device according to any one of embodiments 1-43 or 53-57, comprising a measurement device or sensor adapted to deliver a measurement to at least one of the DDI, patent EID external device and a patient display device.
45. The system configured for changing pre-programmed treatment settings of an implantable medical device according to any one of embodiments 1-44 or 53-57, comprising a food sensor, adapted to measure at least if the patient swallows solid food or is drinking fluid, wherein said food sensor is connected to the control unit of a medical device to cause an action to stretch the stomach after a determined amount of food intake.
46. A system configured for changing pre-programmed treatment settings of an implantable medical device according to any one of embodiments 1-45 or 53-57, wherein the HCP, HCP EID, or HCP via HCP EID are adapted to be providing changed pre-programmed settings of an implant of a human or mammal to:
a communication unit configured to receive such changed pre-programmed settings in an authenticated file, wherein the communication unit is controlled by a first chip, a first circuit, or a first chip and a first circuit, adapted to further provide the authenticated file to:
an decryption unit for decrypting the file received, controlled by a second chip, a second circuit, or a second chip and a second circuit adapted to not be directly in contact with any external device, adapted to be received via:
a narrow secure communication tunnel between the first chip, a first circuit, or a first chip and a first circuit, and the second chip, a second circuit, or a second chip and a second circuit, wherein the communication unit is adapted to only communicate with the decryption unit via the narrow secure communication tunnel, and wherein the decryption unit is adapted to authenticate the HCP provider of the authenticated file and that the data of the file has not been tampered with.
47. A system according to embodiment 46, wherein the authenticated file is adapted to be received indirect by the decryption unit adapted to decrypt the received data and use the decrypted data after the decryption unit has authenticated the HCP, HCP EID, or HCP and HCP EID as originator of the received data of the authenticated file and that the file or data has not been tampered with.
48. A system according to any one of embodiments 46-47, wherein the authenticated file comprises instruction about selected programmed settings of steps of action, wherein use of the decrypted data of the authenticated file comprises instructions to select anyone of the pre-programmed settings of steps of actions of the implant.
49. A system according to any one of embodiments 46-48, wherein the implant is adapted to authenticate, decrypt or authenticate and decrypt the received data using a first key.
50. A system according any one of embodiments 46-49, wherein the HCP private key, patient private key, or HCP private key and patient private key comprising a hardware key.
51. A system according to any one of embodiments 46-49, wherein the HCP private key, patient private key, or HCP private key and patient private key comprising an advanced software key.
52. A system according to any one of embodiments 1-51 or 53-57, wherein authenticated file is adapted to be received by a storage unit of the decryption unit comprising at least one of authenticating information and key related information, related to the HCP, HCP EID or HCP and HCP EID, wherein the decryption unit is adapted to compare stored information with received authenticated file data related to at least one of authentication and decryption.
53. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein the implantable medical device comprises an apparatus for treating obesity of an patient having a stomach with a food cavity, the apparatus comprising:
      a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
      at least one adjustable stretching device adapted to be at least substantially invaginated by a stomach wall portion of the patient with the outer surface of the stretching device resting against the stomach wall and adapted to stretch a portion of stomach wall, and
      a fluid connection device interconnecting the volume filling device and the stretching device.
54. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein the implantable medical device comprises a device for treating obesity of a patient, the device comprising
      at least one operable stretching device (10; 110) implantable in the patient and adapted to stretch a portion of the patient's stomach wall (12), and
      an implantable control unit (42) for automatically controlling the operable stretching device, when the control unit and stretching device are implanted, to stretch the stomach wall portion in connection with the patient eating such that satiety is created,
      wherein the stretching device comprises a first and a second engaging part (10″; 110a; 110b; 210a; 210b), wherein:
      the first part is adapted to be engaged to a first area of the stomach wall, and
      the second part is adapted to be engaged to a second area of the stomach wall, and wherein
      the stretching device is adapted to stretch a portion of the stomach wall between the first area and the second area.
55. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein the implantable medical device comprises an obesity treatment device comprising:
      at least one operable stretching device (10; 10″; 110a; 110b; 410b; 50; 810) implantable in a patient and adapted to stretch a portion of the patient's stomach wall (12) and
      an implantable operation device (90; 42; 54; 40; 217; 218; 452) for operating the stretching device, when implanted, to stretch the stomach wall portion such that satiety is created,
      wherein the operable stretching device is adapted to be placed against the outside of the stomach wall.
56. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device,
wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein the implantable medical device comprises a device for treating obesity of a patient, the device comprising:
      at least one operable stretching device implantable in the patient and adapted to stretch a portion of the patient's stomach wall, and
      an implantable control unit for controlling the operable stretching device, when the control unit and the stretching device are implanted, to stretch the stomach wall portion in connection with the patient eating such that satiety is created,
      wherein the stretching device comprises a first engaging part and a second engaging part, wherein:
      the first engaging part is adapted to be engaged to a first area of the stomach wall, and
      the second engaging part is adapted to be engaged to a second area of the stomach wall, and wherein
      the stretching device is adapted to stretch a portion of the stomach wall between the first area and the second area.
57. A system configured for changing pre-programmed treatment settings of an implantable medical device, when implanted in a patient, from a distant remote location in relation to the patient, the system comprising:
an implantable medical device,
at least one health care provider, HCP, EID external device, and
a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
a reading slot or comparable for the HCP private key device,
a RFID communication, and
other close distance wireless activation communication or electrical direct contact;
wherein said HCP EID external device further comprises at least one wireless transceiver configured for communication with a data infrastructure server, DDI, through a first network protocol,
wherein said system further comprises:
a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 397SE—eHealth_General_Communication_Dual

1. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted.
2. The system according to embodiment 1, wherein the HCP EID external device further comprises a wireless transceiver configured for communication with the implanted medical device through a second network protocol.
3. The system according to embodiment 1 or 2, wherein the HCP private key device is adapted to be provided to the at least one HCP external device via at least one of;
   a reading slot or comparable for the HCP private key device,
   a RFID communication, and
   a close distance wireless activation communication unit, or electrical direct contact.
4. The system according to anyone of the preceding embodiments, wherein the HCP EID external device comprises at least one of:
   reading slot or comparable for the HCP private key device,
   a RFID communication and
   a close distance wireless activation communication unit, or electrical direct contact.
5. The system according to any of the preceding embodiments, wherein the HCP EID external device is adapted to receive a command from a HCP dedicated device to change said pre-programmed treatment steps of the implantable medical device, when implanted, wherein the HCP dedicated device is further adapted to be activated, authenticated, and allowed to perform said command by the HCP providing their private key.
6. The system according to any of the preceding embodiments, wherein at least two of:
   the HCP EID external device,
   the patient EID external device,
   the HCP private key device, and
   the patient private key device,
   are configured for wireless communication using a standard network protocol.
7. The system according to any of the preceding embodiments, wherein at least two of:
   the HCP EID external device,
   the patient EID external device,
   the HCP private key device, and
   the patient private key device,
   are configured for wireless communication using a proprietary network protocol.
8. The system according to any of the preceding embodiments, wherein the patient EID external device is configured to use a first network protocol for communication with the implantable medical device and use a second network protocol for communication with the patient private key device.
9. The system according to any of the preceding embodiments, wherein the patient EID external device is configured to use a first frequency band for communication with the implantable medical device and use a second frequency band for communication with the patient private key device.
10. The system according to any of the preceding embodiments, wherein at least one of the HCP EID external device, the patient EID external device, the HCP private key device, and the patient private key device comprises a Bluetooth transceiver.
11. The system according to any of the preceding embodiments, wherein at least one of the HCP EID external device, the patient EID external device, the HCP private key device, and the patient private key device comprises a UWB transceiver.
12. The system according to embodiment 6, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
13. The system according to any of the preceding embodiments, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
14. The system according to embodiment 13, wherein the second wireless transceiver has an effective range being one of: 2 times, 4 times, 8 time, 20 times, 50 times or 100 times longer than the effective range of the first wireless transceiver.
15. The system according to embodiments 13 or 14, wherein the second wireless transceiver is configured to be disabled to enable wireless communication using the first wireless transceiver.
16. The system according to any of the preceding embodiments, wherein the patient EID external device is configured to allow transfer of data between the EID external device and the implantable medical device on the basis of an authentication of the patient EID external device.
17. The system according to any of the preceding embodiments, wherein the patient EID external device is a wearable patient external device or a handset.
18. The system according to any of the preceding embodiments, wherein the data encrypted by the implantable medical device is related to at least one of:
   a battery status,
   a temperature,
   a time, or
   an error.
19. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
20. The system according to anyone of the preceding embodiments, wherein the patient remote external device and the patient EID external device are an integrated unit.
21. The system according to anyone of the preceding embodiments, wherein the HCP dedicated device and the HCP EID external device are an integrated unit.
22. The system according to anyone of the preceding embodiments, comprising a measurement device or sensor adapted to deliver a measurement to at least one of the DDI, patent EID external device and a patient display device.
23. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.
24. A system configured for changing pre-programmed treatment settings of an implantable medical device according to claim 1-23, wherein the HCP private key and the patient private key are required in the same location normally at the Hospital to activate the HCP EID external device for performing at least one of the actions by the HCP EID external device to at least one of: to change pre-programmed settings, and to update software of the implantable medical device, when the implantable medical device is implanted.

### Aspect 397B—eHealth_General_Communication_Dual

1. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for changing pre-programmed treatment settings of an implantable medical device, when implanted in a patient, by a health care provider, HCP, in the physical presence of the patient, the system comprising:
an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted, wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
the implantable medical device comprises a system for treating a female patient to avoid or promote pregnancy comprising a restriction device adapted to postoperatively restrict and release an oviduct of the patient.
18. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 53-57, wherein the HCP EID external device further comprises a wireless transceiver configured for communication with the implanted medical device through a second network protocol.
23. The system according to any one of embodiments 1-22 or 53-57, wherein the HCP private key device is adapted to be provided to the at least one HCP external device via at least one of;
   a reading slot or comparable for the HCP private key device,
   a RFID communication, and
   a close distance wireless activation communication unit, or electrical direct contact.
24. The system according to any one of embodiments 1-23 or 53-57, wherein the HCP EID external device comprises at least one of:
   reading slot or comparable for the HCP private key device,
   a RFID communication and
   a close distance wireless activation communication unit, or electrical direct contact.
25. The system according to any one of embodiments 1-24 or 53-57, wherein the HCP EID external device is adapted to receive a command from a HCP dedicated device to change said pre-programmed treatment steps of the implantable medical device, when implanted, wherein the HCP dedicated device is further adapted to be activated, authenticated, and allowed to perform said command by the HCP providing their private key.
26. The system according to any one of embodiments 1-25 or 53-57, wherein at least two of:
   the HCP EID external device,
   the patient EID external device,
   the HCP private key device, and
   the patient private key device,
   are configured for wireless communication using a standard network protocol.
27. The system according to any one of embodiments 1-26 or 53-57, wherein at least two of:
   the HCP EID external device,
   the patient EID external device,
   the HCP private key device, and
   the patient private key device,
   are configured for wireless communication using a proprietary network protocol.
28. The system according to any one of embodiments 1-27 or 53-57, wherein the patient EID external device is configured to use a first network protocol for communication with the implantable medical device and use a second network protocol for communication with the patient private key device.
29. The system according to any one of embodiments 1-28 or 53-57, wherein the patient EID external device is configured to use a first frequency band for communication with the implantable medical device and use a second frequency band for communication with the patient private key device.
30. The system according to any one of embodiments 1-29 or 53-57, wherein at least one of the HCP EID external device, the patient EID external device, the HCP private key device, and the patient private key device comprises a Bluetooth transceiver.
31. The system according to any one of embodiments 1-30 or 53-57, wherein at least one of the HCP EID external device, the patient EID external device, the HCP private key device, and the patient private key device comprises a UWB transceiver.
32. The system according to embodiment 26, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
33. The system according to any one of embodiments 1-32 or 53-57, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
34. The system according to embodiment 33, wherein the second wireless transceiver has an effective range being one of: 2 times, 4 times, 8 time, 20 times, 50 times or 100 times longer than the effective range of the first wireless transceiver.
35. The system according to embodiments 33 or 34, wherein the second wireless transceiver is configured to be disabled to enable wireless communication using the first wireless transceiver.
36. The system according to any one of embodiments 1-34 or 53-57, wherein the patient EID external device is configured to allow transfer of data between the EID external device and the implantable medical device on the basis of an authentication of the patient EID external device.
37. The system according to any one of embodiments 1-36 or 53-57, wherein the patient EID external device is a wearable patient external device or a handset.
38. The system according to any one of embodiments 1-37 or 53-57, wherein the data encrypted by the implantable medical device is related to at least one of:
   a battery status,
   a temperature,
   a time, or
   an error.
39. The system according to any one of embodiments 1-38 or 53-57, comprising a master private key device configured to allow issuance of new private key device, wherein the HCP or HCP admin have such master private key device adapted to be able to replace and pair a new patient private key device or HCP private key device into the system, through the HCP EID external device.
40. The system according to any one of embodiments 1-39 or 53-57, wherein the patient remote external device and the patient EID external device are an integrated unit.
41. The system according to any one of embodiments 1-40 or 53-57, wherein the HCP dedicated device and the HCP EID external device are an integrated unit.
42. The system according to any one of embodiments 1-41 or 53-57, comprising a measurement device or sensor adapted to deliver a measurement to at least one of the DDI, patent EID external device and a patient display device.
43. The system according to any one of embodiments 1-42 or 53-57, comprising a food sensor, adapted to measure at least if the patient swallows solid food or is drinking fluid, wherein said food sensor is connected to the control unit of a medical device to cause an action to stretch the stomach after a determined amount of food intake.
44. A system configured for changing pre-programmed treatment settings of an implantable medical device according to claim 1-43 or 53-57, wherein the HCP private key and the patient private key are required in the same location normally at the Hospital to activate the HCP EID external device for performing at least one of the actions by the HCP EID external device to at least one of: to change pre-programmed settings, and to update software of the implantable medical device, when the implantable medical device is implanted.
52. A system according to any one of embodiments 1-51 or 53-57, wherein authenticated file is adapted to be received by a storage unit of the decryption unit comprising at least one of authenticating information and key related information, related to the HCP, HCP EID or HCP and HCP EID, wherein the decryption unit is adapted to compare stored information with received authenticated file data related to at least one of authentication and decryption.
53. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
54. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a device for treating obesity of a patient, the device comprising
   at least one operable stretching device (10; 110) implantable in the patient and adapted to stretch a portion of the patient's stomach wall (12), and an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
55. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
56 A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
57. A system configured for changing pre-programmed treatment settings of an implantable medical device, when implanted in a patient, by a health care provider, HCP, in the physical presence of the patient, the system comprising:
   an implantable medical device,
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted, wherein
the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 397C Implant Programming_Dual_Local

1. A system for updating software of an implantable medical device, when implanted in a patient, by a health care provider, HCP, the system comprising:
a health care provider, HCP, external device, adapted to receive a HCP private key from a HCP private key device,
a patient external device adapted to receive a patient private key from a patient private key device;
a server,
wherein:
the HCP external device is adapted to receive a user command to change said pre-programmed treatment settings, the HCP external device being configured to authorize said user command by receiving the HCP private key device and via the patient private key device, and to transmit said user command and authorization to the server,
the server is adapted to receive said command from the HCP external device and to relay the received command to the patient external device, and
the patient eternal device is adapted to receive the user command and to send said user command to the implanted medical device based on the HCP external device authorization.
2. The system according to embodiment 1, wherein the HCP external device further comprises a wireless transceiver configured for communication with the implanted medical device through a second network protocol.
3. The system according to embodiment 1 or 2, wherein the HCP external device comprises at least one of:
a reading slot or comparable for the HCP private key device,
a RFID communication means, and
a close distance wireless activation communication unit, or electrical direct contact, for receiving the HCP private key from the HCP private key device or for receiving a patient private key from the patient private key device.
4. The system according to any of the preceding embodiments, wherein the HCP external device is adapted to receive a command from a user to change said software of the implantable medical device.
5. The system according to any of the preceding embodiments, wherein at least two of:
the HCP external device,
   the patient external device,
   the HCP private key device, and
the patient private key device,
are configured for wireless communication using a standard network protocol.
6. The system according to any of the preceding embodiments, wherein at least two of:
the HCP EID external device,
the patient external device,
   the HCP private key device, and
the patient private key device,
are configured for wireless communication using a proprietary network protocol.
7. The system according to any of the preceding embodiments, wherein the patient external device is configured to use a first network protocol for communication with the implantable medical device and use a second network protocol for communication with the patient private key device.
8. The system according to any of the preceding embodiments, wherein the patient external device is configured to use a first frequency band for communication with the implantable medical device and use a second frequency band for communication with the server.
9. The system according to any of the preceding embodiments, wherein at least one of the HCP external device, the patient EID external device, the HCP private key device, and the patient private key device comprises a Bluetooth transceiver.
10. The system according to any of the preceding embodiments, wherein at least one of the HCP external device, the patient external device, the HCP private key device, and the patient private key device comprises a UWB transceiver.
11. The system according to embodiment 5, wherein the standard network protocol is one from the list of: Radio Frequency type protocol, RFID type protocol, WLAN type protocol, Bluetooth type protocol, BLE type protocol, NFC type protocol, 3G/4G/5G type protocol, and GSM type protocol.
12. The system according to any of the preceding embodiments, wherein the patient external device comprises a first wireless transceiver for wireless communication with the implantable medical device, and a second wireless transceiver for wireless communication with the server, and wherein the second wireless transceiver has longer effective range than the first wireless transceiver.
13. The system according to any of the preceding embodiments, wherein the patient external device is a wearable external device or a handset.
14. The system according to any of the preceding embodiments, wherein the implantable medical implant is adapted to transmit data to the patient external device, the data being comprising at least one of: a battery status, a temperature, a time, or an error.
15. The system according to anyone of the preceding embodiments, wherein the implantable medical device comprises a measurement device or sensor.
16. The system according to any one of the preceding embodiments, wherein the patient EID is adapted to send said command to the medical implant in response to the HCP external device and the patient external device being within a pre-determined distance, or in response to the HCP external device and the patient external device being connected to each other, or in response to receiving the HCP private key.

### Aspect 398SE—eHealth_General_Communication_Dual

1. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device.
2. The system according to embodiment 1, wherein the wireless transceiver, the remote display portal, and the remote display portal are comprised in the patient remote external device.
3. The system according to embodiments 1 or 2, further comprising the patient display device comprising:
   a supporting application,
   a display which hosts the Remote Display Portal, and
   a patient display device private key.
4. The system according to embodiment 3, wherein the remote display portal is capable of generating a command to be signed by the patient display device private key.
5 The system according to anyone of the preceding embodiments, wherein the patient remote external device is adapted to accept input from the patient via said patient display device through its remote display portal.
6. The system according to anyone of the preceding embodiments, wherein the patient remote external device comprises a graphical user interface arranged on a touch-responsive display exposing buttons to express actuation functions of the implanted medical device.
7. The system according to anyone of the preceding embodiments, configured to allow the patient to actuate the implant at home through the patient remote external device by means of an authorization granted by a patient private key.
8. The system according to embodiment 7, wherein the patient private key comprises at least one of:
   a smart card,
   a keyring device,
   a watch,
   an arm or wrist band,
   a necklace, and
   any shaped device.
9. The system according to anyone of the preceding embodiments, configured to allow the patient to actuate the implantable medical device, when implanted, at home through the patient remote external device, using an authorization granted by the patient private key.
10. The system according to anyone of the preceding embodiments, wherein the system further comprises a patient EID external device comprising at least one of:
   a reading slot or comparable for the patient private key device,
   a RFID communication, and a close distance wireless activation communication, or electrical direct contact.
11. The system according to anyone of the preceding embodiments, wherein the patient EID external device is adapted to be synchronized with the patient remote external device.
12. The system according to anyone of the preceding embodiments, wherein said patient EID external device further comprises at least one of:
   a wireless transceiver configured for communication with the patient, a remote external device, and a wired connector for communication with the patient remote external device.
13. The system according to anyone of the preceding embodiments, wherein the patient EID external device is adapted to generate an authorization to be signed by the patient private key to be installed into at least one of:
   the patient remote external device through the patient EID external device, and
   the implantable medical device.
14. The system according to anyone of the preceding embodiment, comprising a patient display device comprising a supporting application capable of displaying the remote display portal with content delivered from the patient remote external device.
15. The system according to embodiment 14, wherein said remote display portal and patient remote external device are adapted to expose buttons to express the will to actuate the functions of the implanted medical device by the patient through the patient remote external device.
16. The system according to embodiment 14 or 15, wherein the patient display device comprises at least one of:
   a display which hosts the remote display portal, and
   a patient display device private key.
17. The system according to any of embodiments 14-16, wherein said remote display portal is capable of generating a command to be signed by the patient private key.
18. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
19. The system according to anyone of the preceding embodiments, wherein the patient remote external device and the patient EID external device are an integrated unit.
20. The system according to anyone of the preceding embodiments, wherein the HCP dedicated device and the HCP EID external device are an integrated unit.
21. The system according to anyone of the preceding embodiments, comprising a measurement device or sensor adapted to deliver a measurement to at least one of the DDI, patent EID external device and a patient display device.
22. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.

### Aspect 398B—eHealth_General_Communication_Dual

1. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
   the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a system for treating a patient having a disorder related to the patient's intestine, the system comprises an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and the accumulator is adapted to be charged wirelessly with energy and to be arranged so as to supply energy directly or indirectly to at least one energy consuming part of said artificial intestine section.
3. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises a system for treating a female patient to avoid or promote pregnancy comprising a restriction device adapted to postoperatively restrict and release an oviduct of the patient.
18. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device
wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 43-47, wherein the wireless transceiver, the remote display portal, and the remote display portal are comprised in the patient remote external device.
23. The system according to any one of embodiments 1-21 or 43-47, further comprising the patient display device comprising:
a supporting application,
a display which hosts the Remote Display Portal, and
a patient display device private key.
24. The system according to embodiment 23, wherein the remote display portal is capable of generating a command to be signed by the patient display device private key.
25 The system according to anyone of the preceding embodiments or 43-47, wherein the patient remote external device is adapted to accept input from the patient via said patient display device through its remote display portal.
26. The system according to anyone of the preceding embodiments or 43-47, wherein the patient remote external device comprises a graphical user interface arranged on a touch-responsive display exposing buttons to express actuation functions of the implanted medical device.
27. The system according to anyone of the preceding embodiments or 43-47, configured to allow the patient to actuate the implant at home through the patient remote external device by means of an authorization granted by a patient private key.
28. The system according to embodiment 27, wherein the patient private key comprises at least one of:
   a smart card,
   a keyring device,
   a watch,
   an arm or wrist band,
   a necklace, and
   any shaped device.
29. The system according to anyone of the preceding embodiments or 43-47, configured to allow the patient to actuate the implantable medical device, when implanted, at home through the patient remote external device, using an authorization granted by the patient private key.
30. The system according to anyone of the preceding embodiments or 43-47, wherein the system further comprises a patient EID external device comprising at least one of:
   a reading slot or comparable for the patient private key device,
   a RFID communication, and
   a close distance wireless activation communication, or electrical direct contact.
31. The system according to anyone of the preceding embodiments or 43-47, wherein the patient EID external device is adapted to be synchronized with the patient remote external device.
32. The system according to anyone of the preceding embodiments or 43-47, wherein said patient EID external device further comprises at least one of:
   a wireless transceiver configured for communication with the patient, a remote external device, and
   a wired connector for communication with the patient remote external device.
33. The system according to anyone of the preceding embodiments or 43-47, wherein the patient EID external device is adapted to generate an authorization to be signed by the patient private key to be installed into at least one of:
   the patient remote external device through the patient EID external device, and
   the implantable medical device.
34. The system according to anyone of the preceding embodiment or 43-47, comprising a patient display device comprising a supporting application capable of displaying the remote display portal with content delivered from the patient remote external device.
35. The system according to embodiment 34, wherein said remote display portal and patient remote external device are adapted to expose buttons to express the will to actuate the functions of the implanted medical device by the patient through the patient remote external device.
36. The system according to embodiment 34 or 35, wherein the patient display device comprises at least one of:
   a display which hosts the remote display portal, and
   a patient display device private key.
37. The system according to any of embodiments 34-36, wherein said remote display portal is capable of generating a command to be signed by the patient private key.
38. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
39. The system according to anyone of the preceding embodiments or 43-47, wherein the patient remote external device and the patient EID external device are an integrated unit.
40. The system according to anyone of the preceding embodiments or 43-47, wherein the HCP dedicated device and the HCP EID external device are an integrated unit.
41. The system according to anyone of the preceding embodiments or 43-47, comprising a measurement device or sensor adapted to deliver a measurement to at least one of the DDI, patent EID external device and a patient display device.
42. The system according to anyone of the preceding aspects or 43-47, comprising a food sensor, adapted to measure at least if the patient swallows solid food or is drinking fluid, wherein said food sensor is connected to the control unit of a medical device to cause an action to stretch the stomach after a determined amount of food intake.
43. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
44. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
   the implantable medical device comprises a device for treating obesity of a patient, the device comprising
   at least one operable stretching device (10; 110) implantable in the patient and adapted to stretch a portion of the patient's stomach wall (12), and an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
45. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall. 46A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
47. A system configured to change pre-programmed and pre-selected treatment actions of an implantable medical device, when implanted in a patient, by command from the patient, the system comprising:
   an implantable medical device,
   a patient remote external device, comprising
   a wireless transceiver configured for communication with the implantable medical device, when the medical device is implanted, and
   a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 398C eHealth_Pre-Programmed Steps_Patient Remote Control

1. A system for changing pre-programmed steps or pre-selected treatment actions of an implantable medical device, when implanted in a patient, the system comprising:
an implantable medical device,
a patient external device, comprising a wireless transceiver for communication with the implantable medical device, and
a patient display device configured to receive a control interface from the patient external device for receiving user input for actuating a function of the implanted medical device and transmitting the user input to the patient remote external device,
wherein the patient external device is configured to receive the user input form the patient display device, generate a command for the medical implant based on the user input, cryptographically sign the command and transmit the command to the implantable medical device.
2. The system according to embodiment 1, wherein the wireless transceiver and the patient display device are comprised in the patient external device.
3. The system according to embodiments 1 or 2, wherein the patient display device comprises a patient display device private key.
4. The system according to embodiment 3, wherein the patient display device is configured to generate a command signed by the patient display device private key.
5. The system according to anyone of the preceding embodiments, wherein the patient external device comprises a graphical user interface arranged on a touch-responsive display exposing buttons to invoke functions of the implanted medical device.
6. The system according to embodiment 7, wherein the patient private key comprises at least one of: a smart card, a keyring device, a watch, an arm or wrist band, a necklace.
7. The system according to anyone of the preceding embodiments, wherein the patient external device is configured to receive the patient private key for authorizing and in response to receiving the patient private key, transmit a the command to the implantable medical device.
8. The system according to anyone of the preceding embodiments, wherein patient external device comprises at least one of: a reading slot, a RFID communication, and a close distance wireless activation communication, or electrical direct contact, for receiving the patient private key from the patient private key device.
9. The system according to anyone of the preceding embodiments, wherein the patient external device is adapted to generate an authorization for the command to be signed by the patient private key to be installed into at least one of:
   the patient external device, and
   the implantable medical device.
14. The system according to anyone of the preceding embodiment, wherein the patient display device comprises a supporting application capable of displaying the a display portal with content delivered from the patient external device.
15. The system according to embodiment 14, wherein said display portal and patient remote external device are adapted to expose buttons to actuate the functions of the implanted medical device by the patient through the patient remote external device.
16. The system according to embodiment 14 or 15, wherein the patient display device comprises a display for displaying the display portal.
17. The system according to any of embodiments 14-16, wherein said display portal is capable of generating a command to be signed by the patient private key.
18. The system according to any of the preceding embodiments, comprising a master private key device configured to allow issuance of new patient private key device, the master private key device adapted to be able to create a new patient private key for a patient private key device.

### Aspect 399SE—eHealth_General_Implant Information

1. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol.
2. The system according to embodiment 1, wherein the at least one patient EID external device 320′″ is adapted to receive information from the implant, through a second network protocol.
3. The system according to embodiments 1 or 2, comprising the DDI, wherein the DD1 is adapted to receive information from said patient EID external device, and wherein the DDI comprises a wireless transceiver configured for communication with said patient EID external device.
4. The system according to anyone of the preceding embodiments, wherein the patient EID external device is adapted to receive a command relayed by the DDI, to further send the command to the implanted medical device to change said pre-programmed treatment settings of the implanted medical device, and further adapted to be activated and authenticated and allowed to perform said command by the patient providing the patient private key.
5. The system according to anyone of the preceding embodiments, wherein the patient private key device is adapted to provide the patient private key to the patient EID external device by the patient via at least one of; a reading slot or comparable for the patient private key device, an RFID communication or other close distance wireless activation communication, or electrical direct contact.
6. The system according to anyone of the preceding embodiments, said patient EID external device comprising at least one of:
   a reading slot or comparable for the HCP private key device,
   a RFID communication, and
   other close distance wireless activation communication, or direct electrical contact.
7. The system according to anyone of the preceding embodiments, said patient EID external device further comprising at least one wireless transceiver configured for communication with the implanted medical device through a second network protocol.
8. The system according to anyone of the preceding embodiments, comprising the implantable medical device adapted to, when implanted, treat the patient or perform a bodily function.
9. The system according to anyone of the preceding embodiment, wherein the patient private key comprises at least one of:
   a smart card,
   a keyring device,
   a watch, an arm band or wrist band,
   a necklace, and
   any shaped device.
10. The system according to any of the preceding embodiments, wherein at least two of:
   the patient EID external device, the IDD, and
   the patient private key device,
   are configured for wireless communication using a standard network protocol.
11. The system according to any of the preceding embodiments, wherein at least two of:
   the patient EID external device,
   the IDD, and
   the patient private key device,
   are configured for wireless communication using a proprietary network protocol.
12. The system according to any of the preceding embodiments, wherein the patient EID external device is configured to use a first network protocol for communication with the implantable medical device and use a second network protocol for communication with the patient private key device.
13. The system according to any of the preceding embodiments, wherein the patient EID external device is configured to use a first frequency band for communication with the implantable medical device and use a second frequency band for communication with the patient private key device.
14. The system according to any of the preceding embodiments, wherein at least one of the patient EID external device, the patient private key device and the IDD comprises a Bluetooth transceiver.
15. The system according to any of the preceding embodiments, wherein at least one of the patient EID external device, the patient private key device and the IDD comprises a UWB transceiver.
16. The system according to embodiment 10, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
17. The system according to any of the preceding embodiments, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
18. The system according to embodiment 17, wherein the second wireless transceiver has an effective range being one of: 2 times, 4 times, 8 time, 20 times, 50 times or 100 times longer than the effective range of the first wireless transceiver.
19. The system according to embodiments 17 or 18, wherein the second wireless transceiver is configured to be disabled to enable wireless communication using the first wireless transceiver.
20. The system according to any of the preceding embodiments, wherein the patient EID external device is a wearable patient external device or a handset.
21. The system according to any of the preceding embodiments, wherein the data encrypted by the implantable medical device is related to at least one of:
   a battery status,
   a temperature,
   a time, or
   an error.
22. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
23. The system according to anyone of the preceding embodiments, wherein the patient remote external device and the patient EID external device are an integrated unit.
24. The system according to anyone of the preceding embodiments, wherein the HCP dedicated device and the HCP EID external device are an integrated unit.
25. The system according to anyone of the preceding embodiments, comprising a measurement device or sensor adapted to deliver a measurement to at least one of the DDI, patent EID external device and a patient display device.
26. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.

### Aspect 399B1—eHealth_General_Implant Information

1. A system configured for providing information from an implantable medical device, when implanted in a patient, to a distant remote location in relation to the patient, the system comprising:
   an implantable medical device, at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection, wherein said patient EID external device comprises at least one of;
   a reading slot or comparable for the patient private key device, an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system configured for providing information from an implantable medical device, when implanted in a patient, to a distant remote location in relation to the patient, the system comprising:
   an implantable medical device, at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device, an RFID communication, and
   other close distance wireless activation communication or direct electrical contact; wherein said patient EID external device further comprises at least one wireless transceiver configured for communication with the DDI, through a first network protocol, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system configured for providing information from an implantable medical device, when implanted in a patient, to a distant remote location in relation to the patient, the system comprising:
   an implantable medical device, at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of:
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14 A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a system for treating a female patient to avoid or promote pregnancy comprising a restriction device adapted to postoperatively restrict and release an oviduct of the patient.
18. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 47-51, wherein the at least one patient EID external device 320′″ is adapted to receive information from the implant, through a second network protocol.
23. The system according to embodiments 21 or 22 or 47-51, comprising the DDI, wherein the DD1 is adapted to receive information from said patient EID external device, and wherein the DDI comprises
   a wireless transceiver configured for communication with said patient EID external device.
24. The system according to anyone of the preceding embodiments or 47-51, wherein the patient EID external device is adapted to receive a command relayed by the DDI, to further send the command to the implanted medical device to change said pre-programmed treatment settings of the implanted medical device, and further adapted to be activated and authenticated and allowed to perform said command by the patient providing the patient private key.
25. The system according to anyone of the preceding embodiments or 47-51, wherein the patient private key device is adapted to provide the patient private key to the patient EID external device by the patient via at least one of; a reading slot or comparable for the patient private key device, an RFID communication or other close distance wireless activation communication, or electrical direct contact.
26. The system according to anyone of the preceding embodiments or 47-51, said patient EID external device comprising at least one of:
   a reading slot or comparable for the HCP private key device,
   a RFID communication, and
   other close distance wireless activation communication, or direct electrical contact.
27. The system according to anyone of the preceding embodiments or 47-51, said patient EID external device further comprising at least one wireless transceiver configured for communication with the implanted medical device through a second network protocol.
28. The system according to anyone of the preceding embodiments or 47-51, comprising the implantable medical device adapted to, when implanted, treat the patient or perform a bodily function.
29. The system according to anyone of the preceding embodiment or 47-51, wherein the patient private key comprises at least one of:
   a smart card,
   a keyring device,
   a watch, an arm band or wrist band,
   a necklace, and
   any shaped device.
30. The system according to any of the preceding embodiments or 47-51, wherein at least two of:
   the patient EID external device, the IDD, and
   the patient private key device,
   are configured for wireless communication using a standard network protocol.
31. The system according to any of the preceding embodiments or 47-51, wherein at least two of:
   the patient EID external device, the IDD, and
   the patient private key device,
   are configured for wireless communication using a proprietary network protocol.
32. The system according to any of the preceding embodiments or 47-51, wherein the patient EID external device is configured to use a first network protocol for communication with the implantable medical device and use a second network protocol for communication with the patient private key device.
33. The system according to any of the preceding embodiments or 47-51, wherein the patient EID external device is configured to use a first frequency band for communication with the implantable medical device and use a second frequency band for communication with the patient private key device.
34. The system according to any of the preceding embodiments or 47-51, wherein at least one of the patient EID external device, the patient private key device and the IDD comprises a Bluetooth transceiver.
35. The system according to any of the preceding embodiments or 47-51, wherein at least one of the patient EID external device, the patient private key device and the IDD comprises a UWB transceiver.
36. The system according to embodiment 30, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
37. The system according to any of the preceding embodiments or 47-51, wherein the patient EID external device comprises a first wireless transceiver for wireless communication with the implantable medical device, and a second wireless transceiver for wireless communication with the patient private key device, and wherein the second wireless transceiver has longer effective range than the first wireless transceiver.
38. The system according to embodiment 37, wherein the second wireless transceiver has an effective range being one of: 2 times, 4 times, 8 time, 20 times, 50 times or 100 times longer than the effective range of the first wireless transceiver.
39. The system according to embodiments 37 or 38, wherein the second wireless transceiver is configured to be disabled to enable wireless communication using the first wireless transceiver.
40. The system according to any of the preceding embodiments or 47-51, wherein the patient EID external device is a wearable patient external device or a handset.
41. The system according to any of the preceding embodiments or 47-51, wherein the data encrypted by the implantable medical device is related to at least one of:
   a battery status,
   a temperature,
   a time, or
   an error.
42. The system according to any of the preceding embodiments or 47-51, comprising a master private key device configured to allow issuance of new private key device, wherein the HCP or HCP admin have such master private key device adapted to be able to replace and pair a new patient private key device or HCP private key device into the system, through the HCP EID external device.
43. The system according to anyone of the preceding embodiments or 47-51, wherein the patient remote external device and the patient EID external device are an integrated unit.
44. The system according to anyone of the preceding embodiments or 47-51, wherein the HCP dedicated device and the HCP EID external device are an integrated unit.
45. The system according to anyone of the preceding embodiments or 47-51, comprising a measurement device or sensor adapted to deliver a measurement to at least one of the DDI, patent EID external device and a patient display device.
46. The system according to anyone of the preceding aspects or 47-51, comprising a food sensor, adapted to measure at least if the patient swallows solid food or is drinking fluid, wherein said food sensor is connected to the control unit of a medical device to cause an action to stretch the stomach after a determined amount of food intake.
47. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an apparatus for treating obesity of an patient having a stomach with a food cavity, the apparatus comprising:
      a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
      at least one adjustable stretching device adapted to be at least substantially invaginated by a stomach wall portion of the patient with the outer surface of the stretching device resting against the stomach wall and adapted to stretch a portion of stomach wall, and
      a fluid connection device interconnecting the volume filling device and the stretching device.
48. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
49. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
50. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a device for treating obesity of a patient, the device comprising:
      at least one operable stretching device implantable in the patient and adapted to stretch a portion of the patient's stomach wall, and
      an implantable control unit for controlling the operable stretching device, when the control unit and the stretching device are implanted, to stretch the stomach wall portion in connection with the patient eating such that satiety is created,
      wherein the stretching device comprises a first engaging part and a second engaging part, wherein:
      the first engaging part is adapted to be engaged to a first area of the stomach wall, and
      the second engaging part is adapted to be engaged to a second area of the stomach wall, and wherein
      the stretching device is adapted to stretch a portion of the stomach wall between the first area and the second area.
51. A system configured for providing information from an implantable medical device, when implanted in a patient, to a distant remote location in relation to the patient, the system comprising:
   an implantable medical device,
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication with the DDI, through a first network protocol, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 399BSE—eHealth_General_Implant Information

1. A system configured for providing information to an implantable medical device, when implanted in a patient, from a distant remote location in relation to the patient, the system comprising:
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol.

Aspect 399BB—eHealth_General_implant information
1. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a system for treating a female patient to avoid or promote pregnancy comprising a restriction device adapted to postoperatively restrict and release an oviduct of the patient.
18. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of:
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
24. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
25 A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
26. A system configured for providing information to an implantable medical device, when implanted in a patient, from a distant remote location in relation to the patient, the system comprising:
   an implantable medical device,
   at least one patient EID external device 320′″ adapted to transmit information to the implant, further adapted to be activated and authenticated and allowed to transmit said information to the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information about the transmitted information with a server or DDI, through a first network protocol, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 399CSE—eHealth_General_Implant Information

1. A system configured for providing information to an implantable medical device, when implanted in a patient, from a distant remote location in relation to the patient, the system comprising:
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol.

### Aspect 399CB—eHealth_General_Implant Information

1. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a system for treating a female patient to avoid or promote pregnancy comprising a restriction device adapted to postoperatively restrict and release an oviduct of the patient.
18. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.22.

A system configured for providing information to an implantable medical device, when implanted in a patient, from a distant remote location in relation to the patient, the system comprising:
an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein the implantable medical device comprises an apparatus for treating obesity of an patient having a stomach with a food cavity, the apparatus comprising:
      a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
      at least one adjustable stretching device adapted to be at least substantially invaginated by a stomach wall portion of the patient with the outer surface of the stretching device resting against the stomach wall and adapted to stretch a portion of stomach wall, and
      a fluid connection device interconnecting the volume filling device and the stretching device.
23. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
24. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
25 A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
26. A system configured for providing information to an implantable medical device, when implanted in a patient, from a distant remote location in relation to the patient, the system comprising:
   an implantable medical device,
   at least one patient EID external device 320′″ adapted to charge the implant, with energy, further adapted to be activated and authenticated and allowed to charge the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact; wherein said patient EID external device further comprises at least one wireless transceiver configured for communication of log information relating to such charging or implant feedback with a server or DDI, through a first network protocol wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 399D eHealth_Communication_Information from Implant_Remote

1. A system for transmitting information from an implantable medical device, when implanted in a patient, the system comprising:
   an implantable medical device; a patient external device (320′″) adapted to provide an authorization to the implantable medical device for the patient external device to receive information from the implantable medical device, the authorization being based on the patient external device receiving a patient private key, the patient external device being further configured to send the information to a server (330),
   a patient private key device comprising the patient private key adapted to be provided to the patient external device.
2. The system according to embodiment 1, wherein the patient external device comprises at least one of a reading slot, a RFID communication or other close distance wireless communication, or direct electrical connection, for receiving the patient private key device.
3. The system according to embodiment 1 or 2, wherein the authorization is valid for a predetermined period of time, or wherein the authorization is provided at the time of sending the information.
4. The system according to any one of the preceding embodiments, wherein the authorization is provided during charging of the patient external device.
5. The system according to any one of the preceding embodiments, wherein the authorization is provided using a magnetic field, near-field magnetic induction, an electrical field, ultrasound or light, and wherein
   the implantable medical device comprises a receiver for a magnetic field, an electrical field, ultrasound or light.
5. The system according to any one of the preceding embodiments, wherein the patient external device 320′″ is adapted to encrypt the information before sending the information to the server.
6. The system according to any one of the preceding embodiments, wherein the implantable medical device is configured to encrypt the data before sending it to the patient external device.
7. The system according to any one of the preceding embodiments, wherein the patient external device is adapted to transmit information to the server using a first network protocol and receive information from the implant using a second network protocol.
8. The system according to anyone of the preceding embodiments, comprising the implantable medical device, the implantable medical device being adapted to treat the patient or perform a bodily function.
9. The system according to anyone of the preceding embodiment, wherein the patient private key device comprises at least one of a smart card, a keyring device, a watch, an arm band or wrist band, a necklace.
10. The system according to any of the preceding embodiments, wherein at least two of: the patient external device, the server, and the patient private key device, are configured for wireless communication using a standard network protocol.
11. The system according to any of the preceding embodiments, wherein at least two of: the patient external device, the server, and the patient private key device, are configured for wireless communication using a proprietary network protocol.
12. The system according to any of the preceding embodiments, wherein the patient external device is configured to use a first network protocol for communication with the implantable medical device and use a second network protocol for communication with the patient private key device or the server.
13. The system according to any of the preceding embodiments, wherein the patient external device is configured to use a first frequency band for communication with the implantable medical device and use a second frequency band for communication with the patient private key device or the server.
14. The system according to any of the preceding embodiments, wherein at least one of the patient external device, the patient private key device and the server comprises a Bluetooth transceiver.
15. The system according to any of the preceding embodiments, wherein at least one of the patient external device, the patient private key device and the server comprises a UWB transceiver.
16. The system according to embodiment 6, wherein the standard network protocol is one from the list of: Radio Frequency type protocol, RFID type protocol, WLAN type protocol, Bluetooth type protocol,
   BLE type protocol, NFC type protocol, 3G/4G/5G type protocol, and GSM type protocol.
17. The system according to any of the preceding embodiments, wherein the patient external device comprises a first wireless transceiver for wireless communication with the implantable medical device, and a second wireless transceiver for wireless communication with the patient private key device or the server, and wherein the second wireless transceiver has longer effective range than the first wireless transceiver.
18. The system according to embodiment 13, wherein the second wireless transceiver has an effective range being one of: 2 times, 4 times, 8 times, 20 times, 50 times or 100 times longer than the effective range of the first wireless transceiver.
19. The system according to embodiments 17 or 18, wherein the second wireless transceiver is configured to be disabled to enable wireless communication using the first wireless transceiver.
20. The system according to any of the preceding embodiments, wherein the patient external device is a wearable patient external device or a handset.
21. The system according to any of the preceding embodiments, wherein the data encrypted by the implantable medical device is related to at least one of:
   a battery status,
   a temperature,
   a time, or
   an error.
22. The system according to any of the preceding embodiments, comprising a master private key device configured to issue of a new private key device and configured to replace and pair a new patient private key device or HCP private key device into the system, through the HCP external device.
23. The system according to anyone of the preceding embodiments, comprising a measurement device or sensor adapted to deliver a measurement to at least one of the server, patent external device and a patient display device.

### Aspect 330SE eHealth General Communication Housing

1. An external device configured for communication with an implantable medical device implanted in a patient, the external device comprising: a display device,
a housing unit configured to mechanically, disconnectably connect to the display device, the housing unit comprising:
   a first communication unit for receiving communication from the display device, and
a second communication unit for wirelessly transmitting communication to the implantable medical device.
2. The external device according to aspect 1, wherein the external device comprises a handheld electronic device.
3. The external device according to any one of aspects 1 and 2, wherein the external device is configured for communicating with the implantable medical device for changing an operational state of
   the implantable medical device.
4. The external device according to any one of the preceding aspects, wherein the first communication unit is a wireless communication unit for wireless communication with the display device.
5. The external device according to aspect 4, wherein:
the first communication unit is configured to communicate wirelessly with the display device using a first communication frequency,
the second communication unit is configured to communicate wirelessly with the implantable medical device (10) using a second communication frequency, and the first and second communication frequencies are different.
6. The external device according to any one of the preceding aspects, wherein the second communication unit is configured to communicate wirelessly with the implantable medical device (10) using electromagnetic waves at a frequency below 100 kHz.
7. The external device according to any one of the preceding aspects, wherein the second communication unit is configured to communicate wirelessly with the implantable medical device (10) using electromagnetic waves at a frequency below 40 kHz.
8. The external device according to any one of aspects 4-7, wherein the first communication unit is configured to communicate wirelessly with the display device using electromagnetic waves at a frequency above 100 kHz.
9. The external device according to any one of the preceding aspects, wherein:
the first communication unit is configured to communicate with the display device using a first communication protocol,
the second communication unit is configured to communicate wirelessly with the implantable medical device (10) using a second communication protocol, and the first and second communication protocols are different.
10. The external device according to any one of aspects 3-9, wherein the housing unit comprises: a first antenna configured for wireless communication with the display device, and a second antenna configured for wireless communication with the implantable medical device (10).
11. The external device according to any one of aspects 1-3, wherein the first communication unit is a wire-based communication unit for wire-based communication with the display device.
12. The external device according to any one of the preceding aspects, wherein the display device comprises: a first communication unit for communication with the housing unit, and a second communication unit for wireless communication with a second external device.
13. The external device according to aspect 12, wherein the second communication unit of the display device is configured for communicating with the second external device over the Internet.
14. The external device according to any one of aspects 12 and 13, wherein the first communication unit of the display device is a wireless communication unit for wireless communication with the housing unit.
15. The external device according to aspect 14, wherein:
the first communication unit of the display device is configured to communicate wirelessly with the housing unit using a first communication frequency,
the second communication unit of the display device is configured to communicate wirelessly with the second external device using a second communication frequency, and the first and second communication frequencies are different.
16. The external device according to any one of aspects 14 and 15, wherein:
the first communication unit of the display device is configured to communicate wirelessly with the housing unit using a first communication protocol,
the second communication unit of the display device is configured to communicate wirelessly with the second external device using a second communication protocol, and the first and second communication protocols are different.
17. The external device according to any one of aspects 14-16, wherein the display device comprises:
a first antenna configured for wireless communication with the housing, and
a second antenna configured for wireless communication with the second external device.
18. The external device according to any one of aspects 12-13, wherein the first communication unit is a wire-based communication unit for wire-based communication with the housing unit.
19. The external device according to any one of the preceding aspects, wherein the display device is configured to display a user interface to the patient.
20. The external device according to any one of the preceding aspects, wherein the housing unit is configured to transmit information pertaining to the display of the user interface to the display device.
21. The external device according to any one of aspects 19 and 20, wherein the display device is configured to:
receive input pertaining to communication to or from the implantable medical device from the patient, and
transmit communication based on the received input to the housing unit.
22. The external device according to any one of aspects 19-21, wherein the display device comprises a touch screen configured to display the user interface and receive the input from the patient.
23. The external device according to any one of the preceding aspects, wherein the housing unit is configured to display a user interface to the patient.
24. The external device according to any one of the preceding aspects, wherein the first communication unit of the housing unit is configured to receive communication from the implantable medical device pertaining to input from the patient, and wirelessly transmit communication based on the received input to the implantable medical device, using the second communication unit.
25. The external device according to any one of the preceding aspects, wherein the second communication unit of the housing unit is configured for wireless communication with the implantable medical device using a standard network protocol.
26. The external device according to aspect 25, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
27. The external device according to aspect 25, wherein the second communication unit of the housing unit comprises a Bluetooth transceiver.
27. The external device according to any one of the preceding aspects, wherein the second communication unit of the housing unit is configured for wireless communication with the implantable medical device using a proprietary network protocol.
28. The external device according to any one of aspects 25-27, wherein the second communication unit of the housing unit comprises a UWB transceiver.
29. The external device according to any one of aspects 4-28, wherein the first communication unit of the housing unit is configured for wireless communication with the display device using a standard network protocol.
30. The external device according to aspect 29, wherein the standard network protocol is an NFC type protocol.
31. The external device according to any one of aspects 4-28, wherein the first communication unit of the housing unit is configured for wireless communication with the display device using a proprietary network protocol.
32. The external device according to any one of aspects 4-31, wherein a communication range of
the first communication unit of the housing unit is less than a communication range of the second communication unit of the housing unit.
33. The external device according to any one of aspects 14-32, wherein a communication range of
the first communication unit of the display device is less than a communication range of the second communication unit of the display device.
34. The external device according to any one of the preceding aspects, wherein at least one of the housing unit and the display device is configured allow communication between the housing unit and the display device on the basis of a distance between the housing unit and the display device.
35. The external device according to any one of the preceding aspects, wherein at least one of the housing unit and the display device is configured allow communication between the housing unit and the display device on the basis of the housing unit being mechanically connected to the display device.
36. The external device according to any one of the preceding aspects, wherein the housing unit is configured allow communication between the housing unit and the implantable medical device on the basis of a distance between the housing unit and the implantable medical device.
37. The external device according to any one of the preceding aspects, wherein the housing unit further comprises an encryption unit configured to encrypt communication received from the display device.
38. The external device according to aspect 37, wherein the housing unit is further adapted to transmit the encrypted communication, using the second communication unit, to the implantable medical device.
39. The external device according to any one of aspects 14-38, wherein the second communication unit of the display device is configured to be disabled to enable at least one of: communication between the display device and the housing unit, and communication between the housing unit and the implantable medical device.
40. The external device according to any one of the preceding aspects, wherein the display device is a wearable device or a handset.
41. The external device according to aspect 40, wherein the housing unit comprises a case for the wearable device or handset.
42. The external device according to any one of the preceding aspects, wherein the implantable medical device is an implantable medical device configured to exert a force on a body portion of the patient.
43. The external device according to any one of the preceding aspects, wherein the implantable medical device comprises an electrical motor and a controller (300) for controlling the electrical motor.
44. The external device according to any one of aspects 1-41 wherein the implantable medical device comprises at least one of
an external heart compression device,
an apparatus assisting the pump function of a heart of the patient,
an apparatus assisting the pump function comprising a turbine bump placed within a patient's blood
vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of
the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
   an implant configured to exercise a muscle with electrical or mechanical stimulation,
an implant configured for emptying an intestine portion on command,
an operable implant configured to be invaginated in the stomach of the patient to reduce the volume of
the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
   an implant configured for stimulating the ampulla vas deference and creating temporary constriction.
45. A housing unit configured for communication with an implantable medical device (10) when implanted in a patient, the housing unit being configured to mechanically, disconnectably connect to a display device and comprising:
   a first communication unit for receiving communication from the display device, and a second communication unit for wirelessly transmitting communication to the implantable medical device.
46. The housing unit according to aspect 45, wherein the display device is a wearable device or a handset and the housing unit comprises a case for the wearable device or handset.
47. The housing unit according to any one of aspects 45-46, wherein the first communication unit is a wireless communication unit for wireless communication with the display device.
48. The housing unit according to aspect 47, wherein:
the first communication unit is configured to communicate wirelessly with the display device using a first communication frequency,
the second communication unit is configured to communicate wirelessly with the implantable medical device using a second communication frequency, and the first and second communication frequencies are different.
49. The housing unit according to any one of aspects 45-48, wherein the second communication unit is configured to communicate wirelessly with the implantable medical device (10) using electromagnetic waves at a frequency below 100 kHz.
50. The housing unit according to any one of aspects 45-49, wherein the second communication unit is configured to communicate wirelessly with the implantable medical device using electromagnetic waves at a frequency below 40 kHz.
51. The housing unit according to any one of aspects 47-50, wherein the first communication unit is configured to communicate wirelessly with the display device using electromagnetic waves at a frequency above 100 kHz.
52. The housing unit according to any one of aspects 45-51, wherein:
the first communication unit is configured to communicate wirelessly with the display device using a first communication protocol,
the second communication unit is configured to communicate wirelessly with the implantable medical device using a second communication protocol, and
the first and second communication protocols are different.
53. The housing unit according to any one of aspects 47-52, wherein the housing unit comprises:
a first antenna configured for wireless communication with the display device, and
a second antenna configured for wireless communication with the implantable medical device (10).
54. The housing unit according to any one of aspects 45-46, wherein the first communication unit is a wire-based communication unit for wire-based communication with the display device.
55. The housing unit according to any one of aspects 45-54, wherein the housing unit is configured to transmit information pertaining to the display of a user interface to the display device.
56. The housing unit according to any one of aspects 45-55, wherein the housing unit is configured to receive patient input from the display device.
57. The housing unit according to any one of aspects 45-56, wherein the housing unit is configured to display a user interface to the patient.
58. The housing unit according to any one of aspects 45-57, wherein the second communication unit is configured for wireless communication with the implantable medical device using a standard network protocol.
59. The housing unit according to aspect 58, wherein the standard network protocol is one selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
60. The housing unit according to aspect 58, wherein the second communication unit comprises a Bluetooth transceiver.
61. The housing unit according to any one of aspects 45-57, wherein the second communication unit is configured for wireless communication with the implantable medical device using a proprietary network protocol.
62. The housing unit according to any one of aspects 58-61, wherein the second communication unit of the housing unit comprises a UWB transceiver.
63. The housing unit according to any one of aspects 47-62, wherein the first communication unit of the housing unit is configured for wireless communication with the display device using a standard network protocol.
64. The housing unit according to aspect 63, wherein the standard network protocol is an NFC type protocol.
65. The housing unit according to any one of aspects 47-62, wherein the first communication unit of the housing unit is configured for wireless communication with the display device using a proprietary network protocol.
66. The housing unit according to any one of aspects 47-65, wherein a communication range of the first communication unit is less than a communication range of the second communication unit.
67. The housing unit according to any one of aspects 45-66, wherein the housing unit is configured allow communication between the housing unit and the display device on the basis of a distance between the housing unit and the display device.
68. The housing unit according to any one of aspects 45-67, wherein the housing unit is configured allow communication between the housing unit and the display device on the basis of the housing unit being mechanically connected to the display device.
69. The housing unit according to any one of aspects 45-68, wherein the housing unit is configured allow communication between the housing unit and the implantable medical device on the basis of a distance between the housing unit and the implantable medical device.
70. The housing unit according to any one of aspects 45-69, wherein the housing unit further comprises an encryption unit configured to encrypt communication received from the display device.
71. The housing unit according to aspect 70, wherein the housing unit is further adapted to transmit the encrypted communication, using the second communication unit, to the implantable medical device.
72. The housing unit according to aspects 45-71, wherein the minimum bounding box of the housing unit and the display device when mechanically connected, is no more than: 10% wider, 10% longer or 100% higher, than the minimum bounding box of the display device.
73. The housing unit according to aspects 45-72, wherein the housing unit comprises one or more switches configured to, when the housing is not mechanically connected to the display device, be used by the patient.
74. The housing unit according to aspect 73, wherein the switches are at least partly covered by the display device, when the display device is mechanically connected to the housing unit.
75. The housing unit according to any one of aspects 45-74, wherein at least a part of the housing unit is configured to bend to mechanically connect to the display device.
76. The housing unit according to any one of aspects 45-75, wherein at least a part of the housing unit is configured to covers at least one side of the display device.
77. The housing unit according to any one of aspects 45-76, wherein the housing unit is configured to clasp the display device.
78. The housing unit according to any one of aspects 45-76, wherein the housing unit is configured to mechanically connect to the display unit by an attachment device mechanically connected to the housing unit and to the display device.
79. The housing unit according to any one of aspects 45-76, wherein the housing unit comprises a magnet for magnetically attaching the housing unit to the display device.
80. The housing unit according to any one of aspects 45-79, wherein the housing unit is configured to communicate with an implantable medical device configured to exert a force on a body portion of the patient.
81. The external device according to any one of aspects 45-80, wherein the housing unit is configured to communicate with an implantable medical device comprising an electrical motor and a controller (300) for controlling the electrical motor.
82. The external device according to any one of aspects 45-81, wherein the housing unit is configured to communicate with an implantable medical device comprising at least one of
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof, a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
   an implant configured for stimulating the ampulla vas deference and creating temporary constriction.

### Aspect 330B eHealth General Communication Housing

1. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable energized medical device configured to be held in position by a tissue portion of a patient, the medical device comprising:
      a first portion configured to be placed on a first side of the tissue portion, the first portion having a first cross-sectional area in a first plane and comprising a first surface configured to face a first tissue surface of the first side of the tissue portion,
      a second portion configured to be placed on a second side of the tissue portion, the second side opposing the first side, the second portion having a second cross-sectional area in a second plane and comprising a second surface configured to engage a second tissue surface of the second side of the tissue portion, and
      a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
2. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an artificial flow control device implantable in the patient's body and adapted to control flow of the intestinal contents from said reservoir the artificial flow control device comprises at least one pump adapted to act on said intestinal wall so as to reduce the reservoir's volume in order to empty the reservoir.
4. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a system for treating a female patient to avoid or promote pregnancy comprising a restriction device adapted to postoperatively restrict and release an oviduct of the patient.
18. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to embodiment 1-21 or 65-69, wherein the external device comprises a handheld electronic device.
23. The system according to any one of embodiments 1-22 or 65-69, wherein the external device is configured for communicating with the implantable medical device for changing an operational state of
   the implantable medical device.
24. The system according to any one of the preceding embodiments or 65-69, wherein the first communication unit is a wireless communication unit for wireless communication with the display device.
25. The system according to embodiment 24, wherein:
the first communication unit is configured to communicate wirelessly with the display device using a first communication frequency,
the second communication unit is configured to communicate wirelessly with the implantable medical device (10) using a second communication frequency, and
the first and second communication frequencies are different.
26. The system according to any one of the preceding embodiments or 65-69, wherein the second communication unit is configured to communicate wirelessly with the implantable medical device (10) using electromagnetic waves at a frequency below 100 kHz.
27. The system according to any one of the preceding embodiments or 65-69, wherein the second communication unit is configured to communicate wirelessly with the implantable medical device (10) using electromagnetic waves at a frequency below 40 kHz.
28. The system according to any one of embodiments 24-27, wherein the first communication unit is configured to communicate wirelessly with the display device using electromagnetic waves at a frequency above 100 kHz.
29. The system according to any one of the preceding embodiments or 65-69, wherein:
the first communication unit is configured to communicate with the display device using a first communication protocol,
the second communication unit is configured to communicate wirelessly with the implantable medical device (10) using a second communication protocol, and
the first and second communication protocols are different.
30. The system according to any one of embodiments 23-29, wherein the housing unit comprises:
a first antenna configured for wireless communication with the display device, and
a second antenna configured for wireless communication with the implantable medical device (10).
31. The system according to any one of embodiments 1-23 or 65-69, wherein the first communication unit is a wire-based communication unit for wire-based communication with the display device.
32. The system according to any one of the preceding embodiments or 65-69, wherein the display device comprises:
   a first communication unit for communication with the housing unit, and a second communication unit for wireless communication with a second external device.
33. The system according to embodiment 32, wherein the second communication unit of the display device is configured for communicating with the second external device over the Internet.
34. The system according to any one of embodiments 32 and 33, wherein the first communication unit of the display device is a wireless communication unit for wireless communication with the housing unit.
35. The system according to embodiment 34, wherein:
the first communication unit of the display device is configured to communicate wirelessly with the housing unit using a first communication frequency,
the second communication unit of the display device is configured to communicate wirelessly with the second external device using a second communication frequency, and
the first and second communication frequencies are different.
36. The system according to any one of embodiments 34 and 35, wherein:
the first communication unit of the display device is configured to communicate wirelessly with the housing unit using a first communication protocol,
the second communication unit of the display device is configured to communicate wirelessly with the second external device using a second communication protocol, and
the first and second communication protocols are different.
37. The system according to any one of embodiments 34-36, wherein the display device comprises:
a first antenna configured for wireless communication with the housing, and
a second antenna configured for wireless communication with the second external device.
38. The system according to any one of embodiments 32-33, wherein the first communication unit is a wire-based communication unit for wire-based communication with the housing unit.
39. The system according to any one of the preceding embodiments or 65-69, wherein the display device is configured to display a user interface to the patient.
40. The system according to any one of the preceding embodiments or 65-69, wherein the housing unit is configured to transmit information pertaining to the display of the user interface to the display device.
41. The system according to any one of the embodiments 39 and 40, wherein the display device is configured to:
receive input pertaining to communication to or from the implantable medical device from the patient, and
transmit communication based on the received input to the housing unit.
42. The system according to any one of embodiments 39-41, wherein the display device comprises a touch screen configured to display the user interface and receive the input from the patient.
43. The system according to any one of the preceding embodiments or 65-69, wherein the housing unit is configured to display a user interface to the patient.
44. The system according to any one of the preceding embodiments or 65-69, wherein the first communication unit of the housing unit is configured to receive communication from the implantable medical device pertaining to input from the patient, and wirelessly transmit communication based on the received input to the implantable medical device, using the second communication unit.
45. The system according to any one of the preceding embodiments or 65-69, wherein the second communication unit of the housing unit is configured for wireless communication with the implantable medical device using a standard network protocol.
46. The system according to any one of the preceding embodiments or 65-69, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
47. The system according to embodiment 45, wherein the second communication unit of the housing unit comprises a Bluetooth transceiver.
48. The system according to any one of the preceding embodiments or 65-69, wherein the second communication unit of the housing unit is configured for wireless communication with the implantable medical device using a proprietary network protocol.
49. The system according to any one of embodiments 45-48, wherein the second communication unit of the housing unit comprises a UWB transceiver.
50. The system according to any one of embodiments 24-49, wherein the first communication unit of the housing unit is configured for wireless communication with the display device using a standard network protocol.
51. The system according to embodiment 50, wherein the standard network protocol is an NFC type protocol.
52. The system according to any one of embodiments 24-51, wherein the first communication unit of the housing unit is configured for wireless communication with the display device using a proprietary network protocol.
53. The system according to any one of embodiments 24-52, wherein a communication range of
the first communication unit of the housing unit is less than a communication range of the second communication unit of the housing unit.
54. The system according to any one of embodiments 34-53, wherein a communication range of
the first communication unit of the display device is less than a communication range of the second communication unit of the display device.
55. The system according to any one of the preceding embodiments or 65-69, wherein at least one of the housing unit and the display device is configured allow communication between the housing unit and the display device on the basis of a distance between the housing unit and the display device.
56. The system according to any one of the preceding embodiments or 65-69, wherein at least one of the housing unit and the display device is configured allow communication between the housing unit and the display device on the basis of the housing unit being mechanically connected to the display device.
57. The system according to any one of the preceding embodiments or 65-69, wherein the housing unit is configured allow communication between the housing unit and the implantable medical device on the basis of a distance between the housing unit and the implantable medical device.
58. The system according to any one of the preceding embodiments or 65-69, wherein the housing unit further comprises an encryption unit configured to encrypt communication received from the display device.
59. The system according to embodiment 58, wherein the housing unit is further adapted to transmit the encrypted communication, using the second communication unit, to the implantable medical device.
60. The system according to any one of embodiments 34-59, wherein the second communication unit of the display device is configured to be disabled to enable at least one of: communication between the display device and the housing unit, and communication between the housing unit and the implantable medical device.
61. The system according to any one of the preceding embodiments or 65-69, wherein the display device is a wearable device or a handset.
62. The system according to embodiment 61, wherein the housing unit comprises a case for the wearable device or handset.
63. The system according to any one of the preceding embodiments or 65-69, wherein the implantable medical device is an implantable medical device configured to exert a force on a body portion of the patient.
64. The system according to any one of the preceding embodiments or 65-69, wherein the implantable medical device comprises an electrical motor and a controller (300) for controlling the electrical motor.
65. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
66. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
67. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
68. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
69. A system comprising
   an implantable medical device,
   an external device configured for communication with an implantable medical device implanted in a patient, the external device comprising:
a display device,
a housing unit configured to mechanically, disconnectably connect to the display device, the housing unit comprising:
   a first communication unit for receiving communication from the display device, and a second communication unit for wirelessly transmitting communication to the implantable medical device, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 331SE eHealth General Security Module

1. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
         a combination of the response communication and the received communication from the external device.
2. The implantable controller according to claim 1, wherein the security module comprises a set of rules for accepting communication from the central unit.
3. The implantable controller according to claim 2, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
4. The implantable controller according to claim 4, wherein the set of rules comprises a rule stipulating that communication from the central unit is only accepted when the wireless transceiver has been placed in the off-mode for a specific time period.
5. The implantable controller according to any one of the preceding claims wherein the central unit is configured to verify a digital signature of the received communication from the external device.
6. The implantable controller according to claim 4, wherein the set of rules comprises a rule stipulating that communication from the central unit is only accepted when the digital signature of the received communication has been verified by the central unit.
7. The implantable controller according to any one of the preceding claims, wherein the central unit is configured to verify the size of the received communication from the external device.
8. The implantable controller according to claim 7, wherein the set of rules comprises a rule stipulating that communication from the central unit is only accepted when the size of the received communication has been verified by the central unit.
9. The implantable controller according to any one of the preceding claims, wherein:
   the wireless transceiver is configured to receive a message from the external device being encrypted with at least a first and second layer of encryption,
   the central unit is configured to decrypt a first layer of decryption and transmit at least a portion of the message comprising the second layer of encryption to the security model, and
   the security module is configured to decrypt the second layer of encryption and transmit a response communication to the central unit based on the portion of the message decrypted by the security module.
10. The implantable controller according to claim 9, wherein the central unit is configured to decrypt a portion of the message comprising a digital signature, such that the digital signature can be verified by the central unit.
11. The implantable controller according to claim 9, wherein the central unit is configured to decrypt a portion of the message comprising message size information, such that the message size can be verified by the central unit.
12. The implantable controller according to claim 9, wherein the central unit is configured to decrypt a first and second portion of the message, and wherein the first portion comprises a checksum for verifying the authenticity of the second portion.
13. The implantable controller according to any one of claims 9-12, wherein the response communication transmitted from the security module comprises a checksum, and wherein the central unit is configured to verify the authenticity of at least a portion of the message decrypted by the central unit using the received checksum.
14. The implantable controller according to claim 4, wherein the set of rules comprises a rule related to the rate of data transfer between the central unit and the security module.
15. The implantable controller according to any one of claims 9-14, wherein the security module is configured to decrypt a portion of the message comprising a digital signature, encrypted with the second layer of encryption, such that the digital signature can be verified by the security module.
16. The implantable controller according to any one of claims 4-15, wherein the central unit is only capable of decrypting a portion of the receive communication from the external device when the wireless transceiver is placed in the off-mode.
17. The implantable controller according to any one of claims 4-16, wherein the central unit is only capable of communicating the at least one instruction to the implantable medical device when the wireless transceiver is placed in the off-mode.
18. The implantable controller according to any one of the preceding claims, wherein the implantable controller is configured to:
   receive, using the wireless transceiver, a message from the external device comprising a first un-encrypted portion and a second encrypted portion,
   decrypt the encrypted portion, and
   use the decrypted portion to verify the authenticity of the un-encrypted portion.
19. The implantable controller according to claim 18, wherein the central unit is configured to:
   transmit the encrypted portion to the security module,
   receive a response communication from the security module, based on information contained in the encrypted portion being decrypted by the security module,
   and use the response communication to verify the authenticity of the un-encrypted portion.
20. The implantable controller according to any one of claims 18-19, wherein the un-encrypted portion comprises at least a portion of the at least one instruction to the implantable medical device.
21. The implantable controller according to any one of the preceding claims, wherein the implantable controller is configured to:
   receive, using the wireless transceiver, a message from the external device comprising information related to at least one of a physiological parameter of the patient and a physical parameter of the implanted medical device, and use the received information to verify the authenticity of the message.
22. The implantable controller according to claim 21, wherein the physiological parameter of the patient comprises at least one of: a temperature, a heart rate and a saturation value.
23. The implantable controller according to claim 21, wherein the physical parameter of the implanted medical device comprises at least one of a current setting or value of the implanted medical device, a prior instruction sent to the implanted medical device or an ID of the implanted medical device.
24. The implantable controller according to any one of claims 21-23, wherein the portion of the message comprising the information is encrypted, and wherein the central unit is configured to transmit the encrypted portion to the security module and receive a response communication from the security module, based on the information having been decrypted by the security module.
25. The implantable controller according to any one of the preceding claims, wherein the security module comprises a hardware security module comprising at least one hardware-based key.
26. The implantable controller according to claim 25, wherein the hardware-based key corresponds to a hardware-based key in the external device.
27. The implantable controller according to claim 25, wherein the hardware-based key corresponds to a hardware-based key on a key-card connectable to the external device.
28. The implantable controller according to any one of the preceding claims, wherein the security module comprises a software security module comprising at least one software-based key.
29. The implantable controller according to claim 28, wherein the software-based key corresponds to a software-based key in the external device.
30. The implantable controller according to claim 28, wherein the software-based key corresponds to a software-based key on a key-card connectable to the external device.
31. The implantable controller according to any one of the preceding claims, wherein the security module comprises a combination of a software-based key and a hardware-based key.
32. The implantable controller according to any one of the preceding claims, wherein the security module comprises at least one crypto processor.
33. The implantable controller according to any one of the preceding claims, wherein the wireless transceiver is configured to receive communication from a handheld external device.
34. The implantable controller according to any one of the preceding claims, wherein the at least one instruction to the implantable medical device comprises an instruction for changing an operational state of the implantable medical device.
35. The implantable controller according to any one of the preceding claims, wherein the wireless transceiver is configured to communicate wirelessly with the external device using electromagnetic waves at a frequency below 100 kHz.
36. The implantable controller according to claim 35, wherein the wireless transceiver is configured to communicate wirelessly with the external device using electromagnetic waves at a frequency below 40 kHz.
37. The implantable controller according to any one of the preceding claims, wherein:
   the wireless transceiver is configured to communicate wirelessly with the external device using a first communication protocol,
   the central unit is configured to communicate with the security module using a second communication protocol, and
   the first and second communication protocols are different.
38. The implantable controller according to any one of the preceding claims, wherein the wireless transceiver is configured to communicate wirelessly with the external device using a standard network protocol.
39. The implantable controller according to claim 38, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
40. The implantable controller according to any one of claims 1-37, wherein the wireless transceiver is configured to communicate wirelessly with the external device using a proprietary network protocol.
41. The implantable controller according to any one of claims 1-40, wherein the wireless transceiver comprises a UWB transceiver.
42. The external device according to any one of the preceding claims, wherein the security module and the central unit are comprised in a controller.
43. The external device according to claim 42, wherein the wireless transceiver is comprised in the controller.
44. The external device according to any one of the preceding claims, wherein the implantable medical device is an implantable medical device configured to exert a force on a body portion of the patient.
45. The external device according to any one of the preceding claims, wherein the implantable medical device comprises an electrical motor and wherein the controller is configured for controlling the electrical motor.
46. The external device according to any one of claims 1-43 wherein the implantable medical device comprises at least one of
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

### Aspect 331B eHealth General Security Module

1. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device,
wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a system for treating a female patient to avoid or promote pregnancy comprising a restriction device adapted to postoperatively restrict and release an oviduct of the patient.
18. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 66-70, wherein the security module comprises a set of rules for accepting communication from the central unit.
23. The system according to embodiment 22, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
24. The system according to embodiment 23, wherein the set of rules comprises a rule stipulating that communication from the central unit is only accepted when the wireless transceiver has been placed in the off-mode for a specific time period.
25. The system according to any one of the preceding embodiments or 66-70 wherein the central unit is configured to verify a digital signature of the received communication from the external device.
26. The system according to embodiment 24, wherein the set of rules comprises a rule stipulating that communication from the central unit is only accepted when the digital signature of the received communication has been verified by the central unit.
27. The system according to any one of the preceding embodiments or 66-70, wherein the central unit is configured to verify the size of the received communication from the external device.
28. The system according to embodiment 27, wherein the set of rules comprises a rule stipulating that communication from the central unit is only accepted when the size of the received communication has been verified by the central unit.
29. The system according to any one of the preceding embodiments or 66-70, wherein:
   the wireless transceiver is configured to receive a message from the external device being encrypted with at least a first and second layer of encryption,
      the central unit is configured to decrypt a first layer of decryption and transmit at least a portion of the message comprising the second layer of encryption to the security model, and
      the security module is configured to decrypt the second layer of encryption and transmit a response communication to the central unit based on the portion of the message decrypted by the security module.
30. The system according to embodiment 29, wherein the central unit is configured to decrypt a portion of the message comprising a digital signature, such that the digital signature can be verified by the central unit.
31. The system according to embodiment 29, wherein the central unit is configured to decrypt a portion of the message comprising message size information, such that the message size can be verified by the central unit.
32. The system according to embodiment 29, wherein the central unit is configured to decrypt a first and second portion of the message, and wherein the first portion comprises a checksum for verifying the authenticity of the second portion.
33. The system according to any one of embodiments 29-32, wherein the response communication transmitted from the security module comprises a checksum, and wherein the central unit is configured to verify the authenticity of at least a portion of the message decrypted by the central unit using the received checksum.
34. The system according to embodiment 24, wherein the set of rules comprises a rule related to the rate of data transfer between the central unit and the security module.
35. The system according to any one of embodiments 29-34, wherein the security module is configured to decrypt a portion of the message comprising a digital signature, encrypted with the second layer of encryption, such that the digital signature can be verified by the security module.
36. The system according to any one of embodiments 24-35, wherein the central unit is only capable of decrypting a portion of the receive communication from the external device when the wireless transceiver is placed in the off-mode.
37. The system according to any one of embodiments 24-36, wherein the central unit is only capable of communicating the at least one instruction to the implantable medical device when the wireless transceiver is placed in the off-mode.
38. The system according to any one of the preceding embodiments or 66-70, wherein the implantable controller is configured to:
   receive, using the wireless transceiver, a message from the external device comprising a first un-encrypted portion and a second encrypted portion,
   decrypt the encrypted portion, and
   use the decrypted portion to verify the authenticity of the un-encrypted portion.
39. The system according to embodiment 29, wherein the central unit is configured to: transmit the encrypted portion to the security module, receive a response communication from the security module, based on information contained in the encrypted portion being decrypted by the security module, and use the response communication to verify the authenticity of the un-encrypted portion.
40. The system according to any one of embodiments 38-39, wherein the un-encrypted portion comprises at least a portion of the at least one instruction to the implantable medical device.
41. The system according to any one of the preceding embodiments or 66-70, wherein the implantable controller is configured to:
   receive, using the wireless transceiver, a message from the external device comprising information related to at least one of: a physiological parameter of the patient and a physical parameter of the implanted medical device, and use the received information to verify the authenticity of the message.
42. The system according to embodiment 41, wherein the physiological parameter of the patient comprises at least one of: a temperature, a heart rate and a saturation value.
43. The system according to embodiment 41, wherein the physical parameter of the implanted medical device comprises at least one of: a current setting or value of the implanted medical device, a prior instruction sent to the implanted medical device or an ID of the implanted medical device.
44. The implantable controller according to any one of claims 21-23, wherein the portion of the message comprising the information is encrypted, and wherein the central unit is configured to transmit the encrypted portion to the security module and receive a response communication from the security module, based on the information having been decrypted by the security module.
45. The system according to any one of the preceding embodiments, wherein the security module comprises a hardware security module comprising at least one hardware-based key.
46. The system according to embodiment 45, wherein the hardware-based key corresponds to a hardware-based key in the external device.
47. The system according to embodiment 45, wherein the hardware-based key corresponds to a hardware-based key on a key-card connectable to the external device.
48. The system according to any one of the preceding embodiments or 66-70, wherein the security module comprises a software security module comprising at least one software-based key.
49. The system according to embodiment 48, wherein the software-based key corresponds to a software-based key in the external device.
50. The system according to embodiment 48, wherein the software-based key corresponds to a software-based key on a key-card connectable to the external device.
51. The system according to any one of the preceding embodiments or 66-70, wherein the security module comprises a combination of a software-based key and a hardware-based key.
52. The system according to any one of the preceding embodiments or 66-70, wherein the security module comprises at least one crypto processor.
53. The system according to any one of the preceding embodiments or 66-70, wherein the wireless transceiver is configured to receive communication from a handheld external device.
54. The system according to any one of the preceding embodiments or 66-70, wherein the at least one instruction to the implantable medical device comprises an instruction for changing an operational state of the implantable medical device.
55. The system according to any one of the preceding embodiments or 66-70, wherein the wireless transceiver is configured to communicate wirelessly with the external device using electromagnetic waves at a frequency below 100 kHz.
56. The system according to embodiment 55, wherein the wireless transceiver is configured to communicate wirelessly with the external device using electromagnetic waves at a frequency below 40 kHz.
57. The system according to any one of the preceding embodiments or 66-70, wherein:
   the wireless transceiver is configured to communicate wirelessly with the external device using a first communication protocol,
      the central unit is configured to communicate with the security module using a second communication protocol, and
the first and second communication protocols are different.
58. The system according to any one of the preceding embodiments, wherein the wireless transceiver is configured to communicate wirelessly with the external device using a standard network protocol.
59. The system according to embodiment 38, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
60. The system according to any one of embodiments 1-57, wherein the wireless transceiver is configured to communicate wirelessly with the external device using a proprietary network protocol.
61. The system according to any one of embodiments 1-60, wherein the wireless transceiver comprises a UWB transceiver.
62. The system according to any one of the preceding embodiments or 66-70, wherein the security module and the central unit are comprised in a controller.
63. The system according to embodiment 62, wherein the wireless transceiver is comprised in the controller.
64. The system according to any one of the preceding embodiments or 66-70, wherein the implantable medical device is an implantable medical device configured to exert a force on a body portion of the patient.
65. The system according to any one of the preceding embodiments or 66-70, wherein the implantable medical device comprises an electrical motor and wherein the controller is configured for controlling the electrical motor.
66. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
67. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
68. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
69. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
a combination of the response communication and the received communication from the external device, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
70. A system comprising
   an implantable medical device,
   an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
         a combination of the response communication and the received communication from the external device, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 331C—eHealth General Security Module

1. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and
communicate the at least one instruction to the implantable medical device based on a successful decryption or verification of the secure communication.
2. The implantable controller according to embodiment 1, wherein the security module comprises a set of rules for accepting communication from the central unit.
3. The implantable controller according to embodiment 2, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
4. The implantable controller according to embodiment 3, wherein the set of rules comprises a rule stipulating that data from the central unit is only accepted when the wireless transceiver has been placed in the off-mode for a specific time period.
5. The implantable controller according to any one of the preceding embodiments wherein the central unit is configured to verify a digital signature of the received data from the external device and on a positive verification send the received data to the security module.
6. The implantable controller according to embodiment 4, wherein the set of rules comprises a rule stipulating that data from the central unit is only accepted when the digital signature of the received communication has been verified by the central unit.
7. The implantable controller according to any one of the preceding embodiments, wherein the central unit is configured to verify the size of the received data from the external device.
8. The implantable controller according to embodiment 7, wherein the set of rules comprises a rule stipulating that data from the central unit is only accepted when the size of the received data has been verified by the central unit.
9. The implantable controller according to any one of the preceding embodiments, wherein:
the wireless transceiver is configured to receive data from the external device being encrypted with at least a first and second layer of encryption,
the central unit is configured to decrypt a first layer of decryption of the data to obtain a first decrypted data, and transmit the first decrypted data comprising the second layer of encryption to the security model, and
the security module is configured to decrypt the second layer of encryption of the first decrypted data and transmit the at least one instruction to the implantable medical.
10. The implantable controller according to embodiment 9, wherein the central unit is configured to decrypt a portion of the data comprising a digital signature, such that the digital signature can be verified by the central unit.
11. The implantable controller according to embodiment 9, wherein the central unit is configured to decrypt a portion of the data comprising message size information, such that the data size can be verified by the central unit.
12. The implantable controller according to embodiment 9, wherein the central unit is configured to decrypt a first and second portion of the data, and wherein the first portion comprises a checksum for verifying the authenticity of the second portion.
13. The implantable controller according to embodiment 4, wherein the set of rules comprises a rule related to the rate of data transfer between the central unit and the security module.
14. The implantable controller according to any one of embodiments 9-13, wherein the security module is configured to decrypt a portion of the data comprising a digital signature, encrypted with the second layer of encryption, such that the digital signature can be verified by the security module.
15. The implantable controller according to any one of embodiments 4-14, wherein the central unit is only able to decrypt a portion of the data received from the external device when the wireless transceiver is placed in the off-mode.
16. The implantable controller according to any one of embodiments 4-15, wherein the security unit is only able to communicate the at least one instruction to the implantable medical device when the wireless transceiver is placed in the off-mode.
17. The implantable controller according to any one of the preceding embodiments, wherein the implantable controller is configured to:
receive, using the wireless transceiver, a message from the external device comprising a first un-encrypted portion and a second encrypted portion,
decrypt the encrypted portion, and
use the decrypted portion to verify the authenticity of the un-encrypted portion.
18. The implantable controller according to any one of embodiment 17, wherein the un-encrypted portion comprises at least a portion of the at least one instruction to the implantable medical device.
19. The implantable controller according to any one of the preceding embodiments, wherein the implantable controller is configured to:
receive, using the wireless transceiver, a message from the external device comprising information related to at least one of: a physiological parameter of the patient and a physical parameter of the implanted medical device, and
use the received information to verify the authenticity of the message.
20. The implantable controller according to embodiment 19, wherein the physiological parameter of the patient comprises at least one of: a temperature, a heart rate and a saturation value.
21. The implantable controller according to embodiment 19, wherein the physical parameter of the implanted medical device comprises at least one of: a current setting or value of the implanted medical device, a prior instruction sent to the implanted medical device or an ID of the implanted medical device.
22. The implantable controller according to any one of embodiments 19-21, wherein the portion of the message comprising the information is encrypted, and wherein the central unit is configured to transmit the encrypted portion to the security module.
23. The implantable controller according to any one of the preceding embodiments, wherein the security module comprises a hardware security module comprising at least one hardware-based key.
24. The implantable controller according to embodiment 23, wherein the hardware-based key corresponds to a hardware-based key in the external device.
25. The implantable controller according to embodiment 23, wherein the hardware-based key corresponds to a hardware-based key on a key-card connectable to the external device.
26. The implantable controller according to any one of the preceding embodiments, wherein the security module comprises a software security module comprising at least one software-based key.
27. The implantable controller according to embodiment 26, wherein the software-based key corresponds to a software-based key in the external device.
28. The implantable controller according to embodiment 26, wherein the software-based key corresponds to a software-based key on a key-card connectable to the external device.
29. The implantable controller according to any one of the preceding embodiments, wherein the security module comprises a combination of a software-based key and a hardware-based key.
30. The implantable controller according to any one of the preceding embodiments, wherein the security module comprises at least one crypto processor.
31. The implantable controller according to any one of the preceding embodiments, wherein the wireless transceiver is configured to receive communication from a handheld external device.
32. The implantable controller according to any one of the preceding embodiments, wherein the at least one instruction to the implantable medical device comprises an instruction for changing an operational state of the implantable medical device.
33. The implantable controller according to any one of the preceding embodiments, wherein the wireless transceiver is configured to communicate wirelessly with the external device using electromagnetic waves at a frequency below 100 kHz.
34. The implantable controller according to embodiment 33, wherein the wireless transceiver is configured to communicate wirelessly with the external device using electromagnetic waves at a frequency below 40 kHz.
35. The implantable controller according to any one of the preceding embodiments, wherein:
   the wireless transceiver is configured to communicate wirelessly with the external device using a first communication protocol,
      the central unit is configured to communicate with the security module using a second communication protocol, and
the first and second communication protocols are different.
36. The implantable controller according to any one of the preceding embodiments, wherein the wireless transceiver is configured to communicate wirelessly with the external device using a standard network protocol.
37. The implantable controller according to embodiment 36, wherein the standard network protocol is selected from a list comprising:
   RFID type protocol,
   WLAN type protocol,
   Bluetooth type protocol,
   BLE type protocol,
   NFC type protocol,
   3G/4G/5G type protocol, and
   GSM type protocol.
38. The implantable controller according to any one of embodiments 1-35, wherein the wireless transceiver is configured to communicate wirelessly with the external device using a proprietary network protocol.
39. The implantable controller according to any one of embodiments 1-38, wherein the wireless transceiver comprises a UWB transceiver.
40. The implantable controller according to any one of the preceding embodiments, wherein the security module and the central unit are comprised in a controller.
41. The implantable controller according to any one of the preceding embodiments, wherein the central module and the security module are implemented in a respective processor on a chip.
42. The implantable controller according to embodiment 40 or 41, wherein the wireless transceiver is comprised in the controller.
43. The implantable controller according to any one of the preceding embodiments, wherein the implantable medical device is an implantable medical device configured to exert a force on a body portion of the patient.
44. The implantable controller according to any one of the preceding embodiments, wherein the implantable medical device comprises an electrical motor and wherein the controller is configured for controlling the electrical motor.
45. The implantable controller according to any one of embodiments 1-44 wherein the implantable medical device comprises at least one of
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

### Aspect 331CB eHealth General Security Module

1. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and
communicate the at least one instruction to the implantable medical device based on a successful decryption or verification of the secure communication,
the implantable medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable controller for an implantable medical device, the implantable controller comprises:
   a wireless transceiver for communicating wirelessly with an external device,
   a security module, and
   a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. An implantable controller for an implantable medical device, the implantable controller comprises:
   a wireless transceiver for communicating wirelessly with an external device,
   a security module, and
   a central unit configured to be in communication with the wireless transceiver and the security module. wherein:
      the wireless transceiver is configured to receive data from the external device including at least one instruction to the implantable medical device, and transmit the received communication to the central unit,
      the central unit is configured to send the data to the security module, derived from the received communication from the external device, and
      the security module is configured to:
         decrypt at least a portion of the data or verify the authenticity of the data, and communicate the at least one instruction to the implantable medical device based on a successful decryption or verification of the secure communication,
   the implantable medical device comprising: a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An implantable controller for an implantable medical device, the implantable controller comprises:
   a wireless transceiver for communicating wirelessly with an external device,
   a security module, and
   a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein the operative supply of lubricating fluid to the damaged joint is controlled continuously, intermittently, periodically or depending on a physical parameter of the patient, such as a fluid level within the joint.
7. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a device for bone adjustment in a mammal, comprising
two or more anchoring devices for attaching to a bone in said mammal, an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable controller for an implantable medical device, the implantable controller comprises:
   a wireless transceiver for communicating wirelessly with an external device,
   a security module, and
   a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a male sexual impotence treatment prosthesis apparatus, comprising
an operable prosthesis implantable in the cavities of the corpora cavernosa of an impotent patient to provide erect penile condition, when the prosthesis is operated, and
an energy transmission device for wireless transmission of energy from outside the patient's body to inside the patient's body for use in connection with the operation of the prosthesis, when the prosthesis is implanted.
12. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: a system for treating a female patient to avoid or promote pregnancy comprising a restriction device adapted to postoperatively restrict and release an oviduct of the patient.
18. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising: an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
a device for treating obesity of a patient, the device comprising:
   at least one operable stretching device implantable in the patient and adapted to stretch a portion of the patient's stomach wall, and
   an implantable control unit for automatically controlling the operable stretching device, when the control unit and stretching device are implanted, to stretch the stomach wall portion in connection with the patient eating such that satiety is created,
   wherein the stretching device comprises a first and a second engaging part, wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
24. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
an obesity treatment device comprising:
   at least one operable stretching device implantable in a patient and adapted to stretch a portion of the patient's stomach wall, and
   an implantable operation device for operating the stretching device, when implanted, to stretch the stomach wall portion such that satiety is created,
   wherein the operable stretching device is adapted to be placed against the outside of the stomach wall.
25. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
26. An implantable controller for an implantable medical device, the implantable controller comprises:
   a wireless transceiver for communicating wirelessly with an external device,
   a security module, and
a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication,
the implantable medical device comprising:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 432SE—eHealth_Variable_Impedance_b 1

1. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value.
2. The implantable medical device according to aspect 1, wherein the controller is configured to vary the variable impedance in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
6. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
7. The implantable medical device according to aspect 6, wherein the controller is configured to control the variable impedance in response to the pulse pattern deviating from a predefined pulse pattern.
8. The implantable medical device according to aspect 6, wherein the controller is configured to control the switch for switching off the electrical connection between the variable impedance and the coil in response to the pulse pattern deviating from a predefined pulse pattern.
9. The implantable medical device according to any one of the preceding aspects, wherein the measurement unit is configured to measure a temperature in the implantable medical device or in the body of the patient, and wherein the controller is configured to control the first and second switch in response to the measured temperature.
10. The implantable medical device according to any one of the preceding aspects, wherein the variable impedance comprises a resistor and a capacitor.
11. The implantable medical device according to any one of the preceding aspects, wherein the variable impedance comprises a resistor and an inductor.
12. The implantable medical device according to any one of the preceding aspects, wherein the variable impedance comprises an inductor and a capacitor.
13. The implantable medical device according to any one of the preceding aspects, wherein the variable impedance comprises a digitally tuned capacitor.
14. The implantable medical device according to any one of the preceding aspects, wherein the variable impedance comprises a digital potentiometer.
15. The implantable medical device according to any one of the preceding aspects, wherein the variable impedance comprises a variable inductor.
16. The implantable medical device according to any one of the preceding aspects, wherein the variation of the impedance is configured to lower the active power that is received by the receiving unit.
17. The implantable medical device according to any one of the preceding aspects, wherein the variable impedance is placed in series with the coil.
18. The implantable medical device according to any one of aspects 1-16, wherein the variable impedance is placed parallel to the coil.
19. The implantable medical device according to any one of the preceding aspects, further comprising an energy storage unit connected to the receiving unit, and wherein the energy storage unit is configured for storing energy received by the receiving unit.
20. The implantable medical device according to any one of the preceding aspects, further comprising an energy consuming part.
21. The implantable medical device according to aspect 20, wherein the energy consuming part of the implantable medical device is configured to exert a force on a body portion of the patient.
22. The implantable medical device according to aspect 20, wherein the energy consuming part of the implantable medical device comprises an electrical motor and wherein the controller is configured for controlling the electrical motor.
23. The implantable medical device according to aspect 20, wherein the energy consuming part comprises at least one of:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

### Aspect 432B—eHealth_Variable_Impedance_b 1

1. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a system for treating a female patient to avoid or promote pregnancy comprising a restriction device adapted to postoperatively restrict and release an oviduct of the patient.
18. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The implantable medical device according to any one of aspects 1-21 or 43-47, wherein the controller is configured to vary the variable impedance in response to the measured parameter exceeding a threshold value.
23. The implantable medical device according to any one of aspects 1-23 or 43-47, wherein the measurement unit is configured to measure a parameter related to the energy received by the coil over a time period.
24. The implantable medical device according to any one of the preceding aspects or
43-47, wherein the measurement unit is configured to measure a parameter related to a change in energy received by the coil.
25. The implantable medical device according to any one of the preceding aspects or
43-47, wherein the first switch is placed at a first end portion of the coil, and wherein the implantable medical device further comprises a second switch placed at a second end portion of the coil, such that the coil can be completely disconnected from other portions of the implantable medical device.
26. The implantable medical device according to any one of the preceding aspects or
43-47, wherein the receiving unit is configured to receive transcutaneously transferred energy in pulses according to a pulse pattern, and wherein the measurement unit is configured to measure a parameter related to the pulse pattern.
27. The implantable medical device according to aspect 6, wherein the controller is configured to control the variable impedance in response to the pulse pattern deviating from a predefined pulse pattern.
28. The implantable medical device according to aspect 6, wherein the controller is configured to control the switch for switching off the electrical connection between the variable impedance and the coil in response to the pulse pattern deviating from a predefined pulse pattern.
29. The implantable medical device according to any one of the preceding aspects or
43-47, wherein the measurement unit is configured to measure a temperature in the implantable medical device or in the body of the patient, and wherein the controller is configured to control the first and second switch in response to the measured temperature.
30. The implantable medical device according to any one of the preceding aspects or
43-47, wherein the variable impedance comprises a resistor and a capacitor.
31. The implantable medical device according to any one of the preceding aspects or
43-47, wherein the variable impedance comprises a resistor and an inductor.
32. The implantable medical device according to any one of the preceding aspects or
43-47, wherein the variable impedance comprises an inductor and a capacitor.
33. The implantable medical device according to any one of the preceding aspects or
43-47, wherein the variable impedance comprises a digitally tuned capacitor.
34. The implantable medical device according to any one of the preceding aspects or
43-47, wherein the variable impedance comprises a digital potentiometer.
35. The implantable medical device according to any one of the preceding aspects or
43-47, wherein the variable impedance comprises a variable inductor.
36. The implantable medical device according to any one of the preceding aspects or
43-47, wherein the variation of the impedance is configured to lower the active power that is received by the receiving unit.
37. The implantable medical device according to any one of the preceding aspects or
43-47, wherein the variable impedance is placed in series with the coil.
38. The implantable medical device according to any one of aspects 1-21, or 43-47, wherein the variable impedance is placed parallel to the coil.
39. The implantable medical device according to any one of the preceding aspects or
43-47, further comprising an energy storage unit connected to the receiving unit, and wherein the energy storage unit is configured for storing energy received by the receiving unit.
40. The implantable medical device according to any one of the preceding aspects or
43-47, further comprising an energy consuming part.
41. The implantable medical device according to aspect 40, wherein the energy consuming part of the implantable medical device is configured to exert a force on a body portion of the patient.
42. The implantable medical device according to aspect 40, wherein the energy consuming part of the implantable medical device comprises an electrical motor and wherein the controller is configured for controlling the electrical motor.
43. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
control the switch for switching off the electrical connection between the variable impedance and the coil in response to the measured parameter exceeding a threshold value, wherein
the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
44. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
45. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
46. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
47. An implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
   control the variable impedance for varying the impedance and thereby tune the coil based on the measured parameter, and
   control the switch for switching off the electrical connection between the variable impedance and the coil in response to the measured parameter exceeding a threshold value, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 433SE—eHealth_Variable_Impedance_b 2

1. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter.
2. The implantable medical device according to aspect 1, wherein the controller is configured to control the first and second switch in response to the measured parameter exceeding a threshold value.
3. The implantable medical device according to any one of aspects 1 and 2, wherein the measurement unit is configured to measure a parameter related to the energy received by the coil over a time period.
4. The implantable medical device according to any one of the preceding aspects, wherein the measurement unit is configured to measure a parameter related to a change in energy received by the coil.
5. The implantable medical device according to any one of the preceding aspects, wherein the receiving unit is configured to receive transcutaneously transferred energy in pulses according to a pulse pattern, and wherein the measurement unit is configured to measure a parameter related to the pulse pattern.
6. The implantable medical device according to aspect 5, wherein the controller is configured to control the first and second switch in response to the pulse pattern deviating from a predefined pulse pattern.
7. The implantable medical device according to any one of the preceding aspects, wherein the measurement unit is configured to measure a temperature in the implantable medical device or in the body of the patient, and wherein the controller is configured to control the first and second switch in response to the measured temperature.
8. The implantable medical device according to any one of the preceding aspects, further comprising an energy storage unit connected to the receiving unit, and wherein the energy storage unit is configured for storing energy received by the receiving unit.
9. The implantable medical device according to any one of the preceding aspects, further comprising an energy consuming part.
10. The implantable medical device according to aspect 9, wherein the energy consuming part of the implantable medical device is configured to exert a force on a body portion of the patient.
11. The implantable medical device according to aspect 9, wherein the energy consuming part of the implantable medical device comprises an electrical motor and wherein the controller is configured for controlling the electrical motor.
12. The implantable medical device according to aspect 9, wherein the energy consuming part comprises at least one of:
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

### Aspect 433B—eHealth_Variable_Impedance_b 2

1. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter,
wherein
   the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a system for treating a female patient to avoid or promote pregnancy comprising a restriction device adapted to postoperatively restrict and release an oviduct of the patient.
18. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The implantable medical device according to any one of aspects 1-21 or 32-36, wherein the controller is configured to control the first and second switch in response to the measured parameter exceeding a threshold value.
23. The implantable medical device according to any one of aspects 1-22 or 32-36, wherein the measurement unit is configured to measure a parameter related to the energy received by the coil over a time period.
24. The implantable medical device according to any one of the preceding aspects or
32-36, wherein the measurement unit is configured to measure a parameter related to a change in energy received by the coil.
25. The implantable medical device according to any one of the preceding aspects or
32-36, wherein the receiving unit is configured to receive transcutaneously transferred energy in pulses according to a pulse pattern, and wherein the measurement unit is configured to measure a parameter related to the pulse pattern.
26. The implantable medical device according to aspect 5, wherein the controller is configured to control the first and second switch in response to the pulse pattern deviating from a predefined pulse pattern.
27. The implantable medical device according to any one of the preceding aspects or
32-36, wherein the measurement unit is configured to measure a temperature in the implantable medical device or in the body of the patient, and wherein the controller is configured to control the first and second switch in response to the measured temperature.
28. The implantable medical device according to any one of the preceding aspects or
32-36, further comprising an energy storage unit connected to the receiving unit, and wherein the energy storage unit is configured for storing energy received by the receiving unit.
29. The implantable medical device according to any one of the preceding aspects or
32-36, further comprising an energy consuming part.
30. The implantable medical device according to aspect 29, wherein the energy consuming part of the implantable medical device is configured to exert a force on a body portion of the patient.
31. The implantable medical device according to aspect 29, wherein the energy consuming part of the implantable medical device comprises an electrical motor and wherein the controller is configured for controlling the electrical motor.
32. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
33. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
34 An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
35. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
36. An implantable medical device comprising a receiving unit comprising:
at least one coil configured for receiving transcutaneously transferred energy,
a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter, wherein
   the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 434SE—eHealth_Variable_Impedance_b 3

1. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern.
2. The implantable medical device according to aspect 1, further comprising at least one switch placed in series with the coil for switching of the coil, wherein the controller is configured to control the switch to switch of the coil in response to the pulse pattern of the received energy deviating from a predetermined pulse pattern.
3. The implantable medical device according to aspect 1, further comprising a variable impedance electrically connected to the coil, for varying the impedance and thereby tuning the coil, and wherein the controller is configured to control the variable impedance in response to the pulse pattern of the received energy deviating from a predetermined pulse pattern.
4. The implantable medical device according to any one of the preceding aspects, wherein the measurement unit is configured to measure a parameter related to the energy received by the coil over a time period.
5. The implantable medical device according to any one of the preceding aspects, wherein the measurement unit is configured to measure a parameter related to a change in energy received by the coil.
6. The implantable medical device according to any one of the preceding aspects, wherein the measurement unit is configured to measure a temperature in the implantable medical device or in the body of the patient, and wherein the controller is configured to control the first and second switch in response to the measured temperature.
7. The implantable medical device according to any one of the preceding aspects, wherein the first switch is placed at a first end portion of the coil, and wherein the implantable medical device further comprises a second switch placed at a second end portion of the coil, such that the coil can be completely disconnected from other portions of the implantable medical device.
8. The implantable medical device according to aspect 3, wherein the variable impedance comprises a resistor and a capacitor.
9. The implantable medical device according to aspect 3, wherein the variable impedance comprises a resistor and an inductor.
10. The implantable medical device according to aspect 3, wherein the variable impedance comprises an inductor and a capacitor.
11. The implantable medical device according to aspect 3, wherein the variable impedance comprises a digitally tuned capacitor.
12. The implantable medical device according to aspect 3, wherein the variable impedance comprises a digital potentiometer.
13. The implantable medical device according to aspect 3, wherein the variable impedance comprises a variable inductor.
14. The implantable medical device according to any one of aspects 3-12, wherein the variation of the impedance is configured to lower the active power that is received by the receiving unit.
15. The implantable medical device according to any one of aspects 3-13, wherein the variable impedance is placed in series with the coil.
16. The implantable medical device according to any one of aspects 3-13, wherein the variable impedance is placed parallel to the coil.
17. The implantable medical device according to any one of the preceding aspects, further comprising an energy storage unit connected to the receiving unit, and wherein the energy storage unit is configured for storing energy received by the receiving unit.
18. The implantable medical device according to any one of the preceding aspects, further comprising an energy consuming part.
19. The implantable medical device according to aspect 18, wherein the energy consuming part of the implantable medical device is configured to exert a force on a body portion of the patient.
20. The implantable medical device according to aspect 18, wherein the energy consuming part of the implantable medical device comprises an electrical motor and wherein the controller is configured for controlling the electrical motor.
21. The implantable medical device according to aspect 18, wherein the energy consuming part comprises at least one of
an external heart compression device,
an apparatus assisting the pump function of a heart of the patient,
an apparatus assisting the pump function comprising a turbine bump placed within a patient's blood vessel for assisting the pump function of the heart,
an operable artificial heart valve,
an operable artificial heart valve for increasing the blood flow to the coronary arteries.
an implantable drug delivery device,
an implantable drug delivery device for injecting directly into a blood vessel and change the position of the injection site, all from within the patient's body,
an implantable drug delivery device for injecting potency enhancing drugs into an erectile tissue of the patient,
a hydraulic, mechanic, and/or electric constriction implant,
an operable volume filling device,
an operable gastric band,
an operable implant for stretching the stomach wall of the patient for creating satiety,
an implant configured to sense the frequency of the patient ingesting food,
an operable cosmetic implant,
an operable cosmetic implant for adjust the shape and/or size in the breast region of a patient,
an implant controlling medical device for the emptying of a urinary bladder,
an implant hindering urinary leakage,
an implant hindering anal incontinence,
an implant controlling the emptying of fecal matter,
an implant monitoring an aneurysm,
an implant for hindering the expansion of an aneurysm,
an implant lubricating a joint,
an implant for affecting the blood flow to an erectile tissue of the patient,
an implant for simulating the engorgement of an erectile tissue,
an implant with a reservoir for holding bodily fluids,
an implant storing and/or emptying a bodily reservoir or a surgically created reservoir,
an implant communicating with a database outside the body,
an implant able to be programmed from outside the body,
an implant able to be programmed from outside the body with a wireless signal,
an implant treating impotence,
an implant controlling the flow of eggs in the uterine tube,
an implant controlling the flow of sperms in the uterine tube,
an implant controlling the flow of sperms in the vas deferens,
an implant for hindering the transportation of the sperm in the vas deferens,
an implant treating osteoarthritis,
an implant performing a test of parameters inside the body,
an implant controlling specific treatment parameters from inside the body,
an implant controlling bodily parameters from inside the body,
an implant controlling the blood pressure,
an implant controlling the blood pressure by affecting the dilatation of the renal artery,
an implant controlling a drug treatment parameter,
an implant controlling a parameter in the blood,
an implant for adjusting or replacing any bone part of a body of the patient,
an implant replacing an organ of the patient or part of an organ of the patient or the function thereof,
a vascular treatment device,
an implant adapted to move fluid inside the body of the patient,
an implant configured to sense a parameter related to the patient swallowing,
an implant configured to exercise a muscle with electrical or mechanical stimulation,
an implant configured for emptying an intestine portion on command,
an operable implant configured to be invaginated in the stomach of the patient to reduce the volume of the stomach substantially more than the volume of the device,
an implant configured for emptying the urinary bladder from within the patient's body by compressing the bladder,
an implant configured for draining fluid from within the patient's body,
an implant configured for the active lubrication of a joint with an added lubrication fluid,
an implant configured for removing clots and particles from the patient's blood stream,
an implant configured for elongating or straightening a bone in the patient, to reduce scoliosis,
   a device to stimulate the brain for a several position to a focused point,
   an artificial stomach replacing the function of the natural stomach,
an implant configured for adjusting the position of a female's urinary tract or bladder neck,
an implant configured for stimulating the ampulla vas deference and creating temporary constriction.

### Aspect 434B—eHealth_Variable_Impedance_b 3

1. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
9. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a system for treating a female patient to avoid or promote pregnancy comprising a restriction device adapted to postoperatively restrict and release an oviduct of the patient.
18. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The implantable medical device according to any one of aspects 1-21 or 41-45, further comprising at least one switch placed in series with the coil for switching of the coil, wherein the controller is configured to control the switch to switch of the coil in response to the pulse pattern of the received energy deviating from a predetermined pulse pattern.
23. The implantable medical device according to any one of aspects 1-21 or 41-45, further comprising a variable impedance electrically connected to the coil, for varying the impedance and thereby tuning the coil, and wherein the controller is configured to control the variable impedance in response to the pulse pattern of the received energy deviating from a predetermined pulse pattern.
24. The implantable medical device according to any one of the preceding aspects or
41-45, wherein the measurement unit is configured to measure a parameter related to the energy received by the coil over a time period.
25. The implantable medical device according to any one of the preceding aspects or
41-45, wherein the measurement unit is configured to measure a parameter related to a change in energy received by the coil.
26. The implantable medical device according to any one of the preceding aspects or
41-45, wherein the measurement unit is configured to measure a temperature in the implantable medical device or in the body of the patient, and wherein the controller is configured to control the first and second switch in response to the measured temperature.
27. The implantable medical device according to any one of the preceding aspects or
41-45, wherein the first switch is placed at a first end portion of the coil, and wherein the implantable medical device further comprises a second switch placed at a second end portion of the coil, such that the coil can be completely disconnected from other portions of the implantable medical device.
28. The implantable medical device according to aspect 23, wherein the variable impedance comprises a resistor and a capacitor.
29. The implantable medical device according to aspect 23, wherein the variable impedance comprises a resistor and an inductor.
30. The implantable medical device according to aspect 23, wherein the variable impedance comprises an inductor and a capacitor.
31. The implantable medical device according to aspect 23, wherein the variable impedance comprises a digitally tuned capacitor.
32. The implantable medical device according to aspect 23, wherein the variable impedance comprises a digital potentiometer.
33. The implantable medical device according to aspect 23, wherein the variable impedance comprises a variable inductor.
34. The implantable medical device according to any one of aspects 23-32, wherein the variation of the impedance is configured to lower the active power that is received by the receiving unit.
35. The implantable medical device according to any one of aspects 23-33, wherein the variable impedance is placed in series with the coil.
36. The implantable medical device according to any one of aspects 23-33, wherein the variable impedance is placed parallel to the coil.
37. The implantable medical device according to any one of the preceding aspects or
41-45, further comprising an energy storage unit connected to the receiving unit, and wherein the energy storage unit is configured for storing energy received by the receiving unit.
38. The implantable medical device according to any one of the preceding aspects or
41-45, further comprising an energy consuming part.
39. The implantable medical device according to aspect 38, wherein the energy consuming part of the implantable medical device is configured to exert a force on a body portion of the patient.
40. The implantable medical device according to aspect 38, wherein the energy consuming part of the implantable medical device comprises an electrical motor and wherein the controller is configured for controlling the electrical motor.
41. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises A device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises An obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. An implantable medical device comprising a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern, wherein
   the implantable medical device comprises A device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 307B Communication Remote Control

1. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises a system for treating a female patient to avoid or promote pregnancy comprising a restriction device adapted to postoperatively restrict and release an oviduct of the patient.
18. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
   an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiment 1-21, wherein:
the signal provider is an acoustic source configured to provide an acoustic signal as the wake signal.
23. The system according to any one of embodiment 1-21, wherein:
the signal provider is a magnetic source configured to provide a magnetic signal as the wake signal.
24. The system according to any one of embodiment 1-21, wherein:
the sensor is configured to detect the received signal strength of a signal; and the internal control unit is further configured to set the processing unit to the active mode in response to the sensor detecting a signal exceeding a threshold signal strength.
25. The system according to any one of embodiment 1-21, wherein:
the sensor is configured to provide a control signal indicative of a wake signal, the internal control unit is configured to set the processing unit to the active mode in response to the control signal, and the internal control unit is configured to control a supply of energy to the processing unit in response to the control signal.
26. The system according to any one of embodiment 1-21, wherein:
the wake signal comprises a predetermined signal pattern; and the internal control unit is further configured to set the processing unit to the active mode in response to the sensor detecting the predetermined signal pattern.
27. The system according to any one of embodiment 1-21, wherein:
the magnetic source comprises a first coil.
28. The system according to embodiment 27, wherein:
the magnetic source further comprises a second coil arranged perpendicular to the first coil, whereby to collectively provide a substantially even magnetic field.
29. The system according to embodiment 27 or embodiment 28, wherein:
the first coil and/or the second coil is configured to provide a signal as a magnetic field with a frequency of 9 to 315 kilohertz, kHz.
30. The system according to embodiment 29, wherein:
the frequency is less than or equal to 125 kHz, preferably less than 58 kHz.
31. The system according to embodiment 30, wherein:
the frequency is less than 50 kHz, preferably less than 20 kHz, more preferably less than 10 kHz.
32. The system according to any of embodiments 23 to 31, wherein:
the magnetic source comprises a magnet.
33. The system according to embodiment 32, wherein:
the magnet is a permanent magnet.
34. The system according to any of embodiments 23 to 34, wherein:
the magnetic source has an off state in which the magnetic source does provides a magnetic field and an on state in which the magnetic source provides a magnetic field.
35. The system according to embodiment 34 wherein the magnetic source further comprises a shielding means for preventing, when the magnetic source is in the off state, the magnetic source from providing a magnetic field.
36. The system according to any of embodiments 23 to 35, wherein the sensor comprises a hall effect sensor, a fluxgate sensor, an ultra-sensitive magnetic field sensor or a magneto-resistive sensor.
37. The system according to any of embodiments 23 to 36, wherein the sensor comprises a third coil having an iron core.
38. The system according to any one of embodiment 1-21, wherein:
the internal control unit comprises a first communication unit for receiving and/or transmitting data from and/or to the external control unit; and the external control unit comprises a second communication unit for transmitting and/or receiving data to and/or from the internal control unit.
39. The system according to embodiment 38, wherein the sensor is comprised in the first communication unit.
40. The system according to embodiment 38 or embodiment 39, further comprising: a frequency detector communicatively coupled to the internal control unit and configured to detect a frequency for data communication between the first communication unit and the second communication unit.
41. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. A system for controlling a medical implant implanted in a patient, comprising:
an internal control unit adapted to be arranged within the patient's body and communicatively coupled to the medical implant, the internal control unit comprising:
a processing unit having a sleep mode and an active mode, and
a sensor configured to detect a wake signal; and
an external control unit adapted to be arranged outside of the patient's body, the external control unit comprising:
a signal provider configured to provide the wake signal;
wherein the internal control unit is further configured to set the processing unit to the active mode in response to the sensor detecting the wake signal, and wherein the medical implant comprises

A device for treating obesity of a patient, the device comprising:
a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 308B Energy Power-Supply Capacitor

1. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a system for treating a female patient to avoid or promote pregnancy comprising a restriction device adapted to postoperatively restrict and release an oviduct of the patient.
18. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The apparatus according to any one of embodiments 1-21 or 41-45, wherein the discharging from the implantable energy source during startup of the energy consuming part is slower than the energy needed for startup of the energy consuming part.
23. The apparatus according to any one of embodiments 1-21 or 41-45, wherein a maximum energy consumption of the energy consuming part is higher than the maximum energy capable of being delivered by the implantable energy source without causing damage to the implantable energy source, and wherein the energy provider is adapted to deliver an energy burst corresponding to difference between the required energy consumption and the maximum energy capable of being delivered by the implantable energy source.
24. The apparatus according to any one of embodiments 1-21 or 41-45, wherein the implantable energy source is a re-chargeable battery.
25. The apparatus according to any one of embodiments 1-21 or 41-45, wherein the implantable energy source is a solid-state battery.
26. The apparatus according to embodiment 25, wherein the battery is a trionychoid battery.
27. The apparatus according to any one of embodiments 1-21 or 41-45, wherein the implantable energy source is connected to the energy consuming part and configured to power the energy consuming part after it has been started using the energy provider.
28. The apparatus according to any one of embodiments 1-21 or 41-45, wherein the energy provider is a capacitor.
29. The apparatus according to any one of embodiments 1-21 or 41-45, wherein the energy provider is a start capacitor.
30. The apparatus according to any one of embodiments 1-21 or 41-45, wherein the energy provider is a run capacitor.
31. The apparatus according to any one of embodiments 1-21 or 41-45, wherein the energy provider is a dual run capacitor.
32. The apparatus according to any one of embodiments 1-21 or 41-45, further comprising a second energy provider configured to be charged by the implantable energy source and to provide the energy consuming part with electrical power.
33. The apparatus according to any one of embodiments 1-21 or 41-45, wherein the energy provider is a supercapacitor.
34. The apparatus according to any one of embodiments 1-21 or 41-45, wherein the energy consuming part is a motor for operating a device or function of the implant.
35. The apparatus according to any one of embodiments 1-21 or 41-45, wherein the energy consuming part is at least one of:
   a device for providing electrical stimulation to a tissue portion of the body of the patient,
   a CPU for encrypting information
   a transmitting and/or receiving unit for communication with an external unit
   a measurement unit or a sensor
   a data collection unit
   a solenoid
   a piezo-electrical element
   a memory metal unit.
36. The apparatus according to any one of embodiments 1-21 or 41-45, wherein the energy consuming part is motor for powering a hydraulic pump.
37. The apparatus according to any one of embodiments 1-21 or 41-45, wherein the energy consuming part is a feedback unit.
38. The apparatus according to embodiment 37, wherein the feedback unit is a vibrator.
39. The apparatus according to any one of embodiments 1-21 or 41-45, wherein the energy consuming part is configured to operate a valve comprised in the implant.
40. The apparatus according to any one of embodiments 1-21 or 41-45, wherein the energy consuming part is a control unit for controlling at least a part of the implant.
41. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. An apparatus for powering an implant for a human patient, comprising:
an implantable energy source for providing energy to the implant,
an energy provider connected to the implantable energy source and connected to an energy consuming part of the implant, the energy provider being configured to store energy to provide a burst of energy to the energy consuming part,
wherein the energy provider is configured to be charged by the implantable energy source and to provide the energy consuming part with electrical power during startup of the energy consuming part, and wherein the medical implant comprises
a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 310B eHealth Double Encryption

1. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a system for treating a female patient to avoid or promote pregnancy comprising a restriction device adapted to postoperatively restrict and release an oviduct of the patient.
18. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 39-43, wherein the encryption unit is configured to encrypt the data to be transmitted using a second key.
23. The system according to any one of embodiments 1-21 or 39-43, wherein the first key or the second key is implant specific information, a secret key associated with the external device, an identifier of the implant or an identifier of the communication unit.
24. The system according to any one of embodiments 1-21 or 39-43, wherein the second key is a key transmitted by the external device to the internal device.
25. The system according to any one of embodiments 1-21 or 39-43, wherein the second key is a combined key comprising a third key received by the implant form the external device.
26. The system according to any one of embodiments 1-21 or 39-43, wherein the first key is a combined key comprising a fourth key, wherein the fourth key is received by the external device from a verification unit connected to or comprised in the external device.
27. The system according to any one of embodiments 1-21 or 39-43, wherein the verification unit is configured to receive authentication input from a user, for authenticating the communication between the implant and the external device.
28. The system according to embodiment 27, wherein the authentication input is a code.
29. The system according to embodiment 27, wherein the authentication input is based on a biometric technique selected from the list of a fingerprint, a palm vein structure, image recognition, face recognition, iris recognition, a retinal scan, a hand geometry, and genome comparison.
30. The system according to embodiment 29, wherein the verification unit is configured to receive a fingerprint from a fingerprint reader.
31. A system according to any preceding embodiment, wherein the information is broadcasted using a short to mid-range transmitting protocol.
32. A system according to any preceding embodiment, wherein the information is transmitted using at least one of
   Radio Frequency type protocol
   RFID type protocol
   WLAN type protocol
   Bluetooth type protocol
   BLE type protocol
   NFC type protocol
   3G/4G/5G type protocol
   GSM type protocol.
   Bluetooth 5
33. A system according to any one of embodiments 1-21 or 39-43, wherein the internal device comprises a first conductive member and the external device comprises a second conductive member, wherein the first and the second conductive members are configured to transmit the data using the body as a conductor.
34. A system according to any one of embodiments 1-21 or 39-43, wherein the communication unit is configured to encrypt the data before transmitting the data.
35. A system according to embodiment 26 wherein the external device is configured to decrypt the received data and encrypt it before transmitting the data to the third device.
36. A system according to any one of embodiments 1-21 or 39-43, wherein the external device is configured to transmit a request for data to the communication unit, and the communication unit is configured to in response to a request for data transmit the data to the external device.
37. A system according to any one of embodiments 1-21 or 39-43, wherein the communication unit further is configured to broadcast the information periodically.
38. A system according to any one of embodiments 1-21 or 39-43, further comprising an internal control unit configured to cause the communication unit to broadcast the information in response to a second parameter being above a predetermined threshold.
39. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
40. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
41. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
42. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
43. A system comprising:
an implant comprising: a communication unit configured to transmit data from the body of the patient to an external device, and
an encryption unit for encrypting the data to be transmitted, and
an external device configured to receive the data transmitted by the communication unit, encrypt the received data using a first key and transmit the encrypted received data to a third device, wherein the implant comprises
a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 311B Communication Remote Control

1. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the joint.
7. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a system for treating a female patient to avoid or promote pregnancy comprising a restriction device adapted to postoperatively restrict and release an oviduct of the patient.
18. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 38-42, wherein the internal controller is configured to verify the integrity of the first set of instructions using a cyclic redundancy check.
23. The system according to any one of embodiments 1-21 or 38-42, wherein the cryptographic hash or metadata comprises a cryptographic hash, and wherein the internal controller is configured to verifying the integrity of the first set of instructions by:
   calculating a second cryptographic hash for the received first set of instructions using a same cryptographic hash algorithm as the processor, and
   determining that the first set of instructions has been correctly received based on that the cryptographic hash and the second cryptographic hash are equal.
24. The system according to embodiment 23, wherein the cryptographic hash algorithm comprises one of
25. The system according to any of embodiments 23-24, wherein the cryptographic hash is a signature obtained by using a private key of the implant, and wherein the internal controller is configured to verifying the first set of instructions by the signature using a public key corresponding to the private key.
26. The system according to any of embodiments 23-25, wherein the cryptographic hash or metadata comprises a metadata, and wherein the internal controller is configured to verifying the integrity of the data by:
   obtaining a second metadata for the received first set of instructions, and
   determining that the first set of instructions has been correctly received based on that metadata and the second metadata are equal.
27. The method according to embodiment 26, wherein the metadata comprises: a length of the data, a timestamp,
28. The system according to any of embodiments 21-27, wherein the external device is separate from the second external device.
29. The system according to any of embodiments 21-28, wherein the internal controller is configured to communicate with the second external device using a different protocol than a protocol used for communication with the external device.
30. The system according to any of embodiments 21-29, wherein the internal communication unit comprises a wireless transceiver for communication with the external device, and a conductive member for communicating with the second external device, wherein the second external device comprises a second conductive member.
31. The system according to embodiment 30, wherein the communication between the internal communication unit and the second external device is performed using the patient's body as a conductor.
32. The system according to any of embodiments 21-31, wherein the internal controller is configured to transmit information relating to the received first set of instructions to the external device, and the external device is configured to confirm that the information relates to the first set of instructions transmitted by the external device.
33. The system according to any of embodiments 21-32, wherein the internal controller is configured to: calculating a second cryptographic hash for the first set of instructions, comparing the second cryptographic hash with the first cryptographic hash, determining that the first set of instructions are authentic based on that the second cryptographic hash is equal to the first cryptographic hash, and upon verification of the authenticity of the first set of instructions, storing them at the implant.
34. The system according to any of embodiments 21-33, wherein the external device is configured to transmit the first set of instructions, and wherein the first set of instructions comprises a cryptographic hash corresponding to a previous set of instructions.
35. The system according to any of embodiments 21-34, wherein the internal controller is connected to or comprising a first sensor adapted to obtain a measurement of a parameter relating to the body of the patient, the external device is connected to or comprising a second sensor adapted to obtain a measurement of the parameter relating to the body of the patient, wherein the first set of instructions comprises the second measurement, and wherein the internal controller is configured to verify the authenticity of the first set of instructions at least based on a comparison of the first and second measurements.
36. The system according to embodiment 35, wherein the first and second parameters relate to a pulse of the patient, a respiration rate of the patient, a temperature of the patient, a sound of the patient, or a physical movement of the patient.
37. The system according to any of embodiments 35-36, wherein the measured parameter by the external device is provided with a timestamp, and the measured parameter measured by the implant is provided with a timestamp, wherein the comparison of the parameter measured at the implant to the parameter measured by the external device comprises comparing the timestamp of the measured parameter received from the implant to the timestamp of the measured parameter by the external device.
38. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
39. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a device for treating obesity of a patient, the device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
40. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
41. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
42. A system for communication instructions, the system comprising:
an implant adapted to be implanted in a patient, the implant comprising an active unit, an internal communication unit and an internal controller,
an external device comprising an external communication unit configured to transmit a first set of instructions to the internal communication unit over a first communications connection,
a second external device comprising a third communication unit configured to transmit a first cryptographic hash to the internal communication unit,
wherein the internal controller is configured to receive, via the internal communication unit, the first set of instructions and the first cryptographic hash and verify the integrity of the first set of instructions based on the first cryptographic hash, and wherein the active portion comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 312B eHealth Programming Predefined Steps

1. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
   the operative supply of lubricating fluid to the damaged joint is controlled continuously, intermittently, periodically or depending on a physical parameter of the patient, such as a fluid level within the Joint.
7. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a male sexual impotence treatment prosthesis apparatus, comprising
   an operable prosthesis implantable in the cavities of the corpora cavernosa of an impotent patient to provide erect penile condition, when the prosthesis is operated,
   characterized by an energy transmission device for wireless transmission of energy from outside the patient's body to inside the patient's body for use in connection with the operation of the prosthesis, when the prosthesis is implanted.
12. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a device for treatment or monitoring of an aneurysm, comprising
   an implantable member adapted to hold fluid, the member being adapted to be placed in connection with a blood vessel having the aneurysm and to exercise a pressure on the aneurysm,
   a control unit adapted to control pressure adjustments of the implantable member.
13. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises a system for treating a female patient to avoid or promote pregnancy comprising a restriction device adapted to postoperatively restrict and release an oviduct of the patient.
18. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The implant according to any one of embodiments 1-21 or 41-45, wherein the predefined program steps comprise setting a variable related to a pressure, a time, a minimum or maximum temperature, a current, a voltage, an intensity, a frequency, an amplitude of electrical stimulation, a feedback, a post-operative mode or a normal mode, a catheter mode, a fibrotic tissue mode, an time open after urination, a time open after urination before bed-time.
23. The implant according to any one of embodiments 1-21 or 41-45, wherein the verification function is configured to reject the update in response to the update comprising program steps not comprised in the set of predefined program steps.
24. The implant according to any one of embodiments 1-21 or 41-45, wherein the verification function is configured to allow the update in response to the update only comprising program steps comprised in the set of predefined program steps.
25. The implant according to any one of embodiments 1-21 or 41-45, wherein the internal communication unit is configured to communicate with the external device via a first wireless connection for receiving the update to the second control program, and a second connection for performing an authentication of the communication with the external device.
26. The implant according to embodiment 25, wherein the second connection is a wireless short-range connection.
27. The implant according to embodiment 25 or 26, wherein the authentication second connection is an electrical connection using the patient's body as a conductor
28. The implant according to any one of embodiments 1-21 or 41-45, wherein the internal computing unit is further configured to, upon verification, installing the update.
29. The implant according to any one of embodiments 1-21 or 41-45, wherein the internal computing unit has a sleep mode and an active mode, and the implant further comprises a sensor configured to detect a wake signal, and wherein the implant is configured to in response to a detected wake signal set the internal computing unit to the active mode.
30. The implant according to embodiment 29, wherein sensor is configured to detect an acoustic signal as wake signal or wherein the sensor is configured to detect a magnetic signal as the wake signal
31. The implant according to any of embodiments 29-30, wherein the sensor is configured to detect the received signal strength of a signal; and the implant is further configured to set the internal computing unit to the active mode in response to the sensor detecting a signal exceeding a threshold signal strength.
32. The implant according to any of embodiments 29-31, further comprising a second internal computing unit, and wherein the implant is configured to set the internal computing unit to the active mode via the second internal computing unit.
33. The implant according to any of embodiments 29-32, wherein the internal computing unit in the sleep mode is substantially without power, and wherein setting the internal computing unit in the active mode comprises providing the internal computing unit with power.
34. The implant according to embodiment 33, wherein the implant comprises an energy controller for controlling the power supplied to the internal computing unit.
35. The implant according to embodiment 34, wherein the sensor is configured to provide the energy controller with a second wake signal in response to detecting the wake signal, and wherein the energy controller is configured to set the computing unit in the active mode in response to the second wake signal.
36. The implant according to any one of embodiments 1-21 or 41-45, wherein the sensor is configured to detect the received signal strength of a signal; and the internal control unit is further configured to set the internal computing unit to the active mode in response to the sensor detecting a signal exceeding a threshold signal strength.
37. The implant according to any one of embodiments 1-21 or 41-45, wherein the wake signal comprises a predetermined signal pattern; and the implant is further configured to set the processing unit to the active mode in response to the sensor detecting the predetermined signal pattern.
38. The implant according to any one of embodiments 1-21 or 41-45, wherein the sensor is a hall effect sensor, a fluxgate sensor, an ultra-sensitive magnetic field sensor or a magneto-resistive sensor.
39. The implant according to any one of embodiments 1-21 or 41-45, wherein the sensor comprises a third coil having an iron core.
40. The implant according to any one of embodiments 1-21 or 41-45, wherein the sensor is comprised in the internal communication unit.
41. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising:
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. An implant comprising:
an internal computing unit configured to control a function of said implant, said internal computing unit comprises an internal memory configured to store:
i. a first control program for controlling the internal computing unit, and
ii. a second, configurable or updatable, with predefined program steps, control program for controlling said function of said implant,
iii. a set of predefined program steps for updating the second control program,
an internal communication unit connected to said internal computing unit and configured to communicate with an external device, wherein said internal computing unit is configured to receive an update to the second control program via said internal communication unit, and
a verification function of, connected to, or transmitted to said internal computing unit, said verification function being configured to verify that the received update to the second control program comprises program steps comprised in the set of predefined program steps, wherein the implant further comprises an implantable energized medical device configured to be held in position by a tissue portion of a patient, the medical device comprising a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 313B eHealth Watchdog

1. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
   an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
an implantable lubrication device comprising:
a reservoir that stores a lubricating fluid and,
a fluid connection that introduces the stored lubricating fluid into a damaged joint when the lubrication device is implanted in a patient's body.
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a male sexual impotence treatment prosthesis apparatus, comprising
   an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
12. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
13. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a system for treating a female patient to avoid or promote pregnancy comprising a restriction device adapted to postoperatively restrict and release an oviduct of the patient.
18. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to anyone of embodiment 1-21 or 41-45, wherein the first control program comprises a second reset function for resetting the timer of the first reset function.
23. The system according to anyone of embodiment 1-21 or 41-45, wherein the first reset function comprises a timer and the second reset function is configured to reset the timer.
24. The system according to anyone of embodiment 1-21 or 41-45, wherein the reset function comprises a first reset function and a second reset function, wherein the first reset function is configured to trigger a corrective function for correcting the first control program, and wherein the second reset function is configured to restart the first control program after the corrective function has been triggered.
25. The system according to anyone of embodiment 1-21 or 41-45, wherein the first or second reset function is configured to invoke a hardware reset by activating an internal or external pulse generator which is configured to create a reset pulse for the internal computing unit or the first control program.
26. The system according to anyone of embodiment 1-21 or 41-45, wherein the internal computing unit is configured to have an active mode and a sleep mode, and wherein the first reset function is configured to have an active mode and a sleep mode corresponding to the active mode and the sleep mode of the internal computing unit.
27. The system according to anyone of embodiment 1-21 or 41-45, further comprising a sensor for measuring a physiological parameter of the patient or a parameter of the implant, and wherein the sensor is configured to invoke the reset function in response to the parameter being above or below a predetermined value.
28. The system according to anyone of embodiment 1-21 or 41-45, wherein the sensor is a pressure sensor adapted to measure a pressure in a part of the implant.
29. The system according to anyone of embodiment 1-21 or 41-45, wherein the pressure sensor is configured to measure a pressure in a reservoir or a restriction device of the implant.
30. The system according to anyone of embodiment 1-21 or 41-45, wherein the sensor is a pressure sensor adapted to measure a pressure in an organ of the patient's body.
31. The system according to anyone of embodiment 1-21 or 41-45, wherein the reset function is configured to be invoked by an electrical reset pulse, and wherein the sensor is adapted to invoke the reset function by activating an internal or external pulse generator which is configured to create a reset pulse for the reset function.
32. The system according to anyone of embodiment 1-21 or 41-45, wherein the physiological parameter of the patient or a parameter of the implant is a temperature.
32. The system according to anyone of embodiment 1-21 or 41-45, wherein the reset function comprises invoking a second control program comprising a safety measure.
33. The system according to anyone of embodiment 1-21 or 41-45, wherein the safety measure comprises controlling a function of the implant.
34. The system according to anyone of embodiment 1-21 or 41-45, wherein the internal computing unit is configured to invoke the reset function periodically.
35. The system according to anyone of embodiment 1-21 or 41-45, wherein periodically comprises every 24 hours.
36. The system according to anyone of embodiment 1-21 or 41-45, wherein the internal computing unit further comprises a monitoring function for monitoring a function of the implant or the first control program, and wherein the reset function is configured to in response to an incorrect or absent response for the monitoring program, reset or restart the first control program.
37. The system according to anyone of embodiment 1-21 or 41-45,
wherein the internal computing unit has an active mode and a sleep mode, the sleep mode having a lower energy consumption than the active mode, and
wherein the implant further comprises an internal control unit connected to the internal computing unit and adapted to control the mode of the internal computing unit.
38. The system according to anyone of embodiment 1-21 or 41-45,
wherein the implant further comprises a second sensor for measuring a physiological parameter of the patient or a parameter of the implant, the second sensor being connected to the internal control unit, and
wherein, in response to a sensor measurement differing from, exceeding or being less than a predetermined value, setting the internal computing unit in the active mode.
39. The system according to anyone of embodiment 1-21 or 41-45, wherein the sensor is configured to measure the physical parameter periodically.
40. The system according to anyone of embodiment 1-21 or 41-45, wherein the sensor and the second sensor is the same sensor.
41. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. A system comprising an implant comprising:
an internal processor comprising:
a first control program for controlling a function of the implant, and
   a first reset function, said first reset function being configured to restart or reset said first control program in response to:
a timer of the first reset function has not been reset, or a malfunction in the first control program
   the system further comprising a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 314B eHealth Logging

1. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
   an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second communication channel, the first and second communication channels being different communication channels
   the system further comprising
a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second communication channel, the first and second communication channels being different communication channels
   the system further comprising
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second communication channel, the first and second communication channels being different communication channels
   the system further comprising an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
   communication channel, the first and second communication channels being different communication channels
   the system further comprising
a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an implantable lubrication device comprising:
   a reservoir that stores a lubricating fluid and,
   a fluid connection that introduces the stored lubricating fluid into a damaged joint when the lubrication device is implanted in a patient's body.
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising a male sexual impotence treatment prosthesis apparatus, comprising
   an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
12. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
13. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising a system for treating a female patient to avoid or promote pregnancy comprising a restriction device adapted to postoperatively restrict and release an oviduct of the patient.
18. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21v, wherein the update or configuration comprises a set of instructions for the control program.
23. The system according to any one of embodiments 1-21, wherein the steps comprise a subset of a set of predefined steps.
24. The system according to any one of embodiments 1-21 or 37-41, wherein the second external device is configured to confirm that the update or configuration is correct based on the received logging data.
25. The system according to any one of embodiments 1-21 or 37-41, wherein the logging data is related to the receipt of the update or configuration, and the internal computing unit is configured to install the update or configuration in response to receipt of a confirmation that the logging data relates to a correct set of instructions.
26. The system according to any one of embodiments 1-21 or 37-41, wherein the logging data is related to the installation of the update or configuration, and wherein the internal computing unit is configured to activate the installation in response to a confirmation that the update or configuration is correct.
27. The system according to any one of embodiments 1-21 or 37-41, wherein the update or configuration comprises a plurality of steps, and the update or configuration is received by the internal computing unit in two or more sub steps.
28. The system according to any one of embodiments 1-21 or 37-41, further comprising a sensation generator adapted to create a sensation detectable by the user.
29. The system according to any one of embodiments 1-21 or 37-41, wherein the internal computing unit is configured to cause the sensation generator to create a sensation detectable by the user in response to the update or configuration being received, in response to the update or configuration being installer or in response to the update or configuration being confirmed.
30. The system according to any one of embodiments 1-21 or 37-41, wherein the sensation generator is a vibrator or a speaker.
31. The system according to any one of embodiments 1-21 or 37-41, wherein the configuration or update comprises a value for a predetermined parameter.
32. The system according to any one of embodiments 1-21 or 37-41, wherein the configuration or update comprises a step from a set of predetermined steps.
33. The system according to any one of embodiments 1-21 or 37-41, wherein communication over the first communication channel is performed using a first network protocol, and communication over the second communication channel is performed using a second network protocol, the first and second protocols being different.
34. The system according to any one of embodiments 1-21 or 37-41, wherein the network protocol is one from the list of:
   Radio Frequency type protocol
   RFID type protocol
   WLAN type protocol
   Bluetooth type protocol
   BLE type protocol
   NFC type protocol
   3G/4G/5G type protocol
   GSM type protocol.
35. The system according to any one of embodiments 1-21 or 37-41, wherein the second network protocol is one from the list of:
   Radio Frequency type protocol
   RFID type protocol
   WLAN type protocol
   Bluetooth type protocol
   BLE type protocol
   NFC type protocol
   3G/4G/5G type protocol
   GSM type protocol.
36. The system according to any one of embodiments 1-21 or 37-41, wherein the second communication channel is an electrical connection.
37. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
39. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
40. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
   the system further comprising
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
41. A system comprising an implant adapted for communication with a first external device and a second external device, when the implant is adapted to be implanted in a patient, the implant comprising:
a communication unit comprising a wireless receiver configured to receive data from the first external device, and a transmitter configured to transmit data to the second external device,
an internal computing unit comprising an updatable control program for controlling a function of said implant, the internal computing unit being connected to the communication unit, and being configured to receive an update or a configuration to the updatable control program from the first external via the communication unit, and the internal computing unit being configured to, when updating the control program, transmit logging data relating to the update to the second external device, and
wherein the communication unit is configured to receive data from the first external device via a first communication channel and transmit data to the second external device via a second
communication channel, the first and second communication channels being different communication channels
the system further comprising
a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 315B eHealth Sleeping Internal Control Unit

1. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
   an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an implantable lubrication device comprising:
   a reservoir that stores a lubricating fluid and,
a fluid connection that introduces the stored lubricating fluid into a damaged joint when the lubrication device is implanted in a patient's body.
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising a male sexual impotence treatment prosthesis apparatus, comprising
   an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
12. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
13. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising a system for treating a female patient to avoid or promote pregnancy comprising a restriction device adapted to postoperatively restrict and release an oviduct of the patient.
18. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to anyone of embodiment 1-21 or 41-45, the sensor is configured to measure periodically.
23. The implant according to anyone of embodiment 1-21 or 41-45, wherein the sensor is a mechanical sensor.
24. The implant according to anyone of embodiment 1-21 or 41-45, wherein the sensor comprises a pressure sensor, a piezoelectric sensor, or a bimetal.
25. The implant according to anyone of embodiment 1-21 or 41-45, wherein:
the sensor is configured to measure a physiological parameter of the patient; and the sensor is a pressure sensor.
26. The implant according to anyone of embodiment 1-21 or 41-45, wherein:
the pressure sensor is adapted to measure a pressure in one or more of: an organ of a patient; a reservoir; and a restriction device.
27. The system according to anyone of embodiment 1-21 or 41-45, wherein:
the sensor is configured to measure a parameter of the implant; and the sensor is adapted to measure one or more of:
   a battery status of a battery of the implant; and
   a temperature of the implant.
28. The system according to anyone of embodiment 1-21 or 41-45, wherein the sensor is an analog sensor or a digital sensor.
29. The system according to anyone of embodiment 1-21 or 41-45, further comprising a sensation generator configured to, upon request, generate a sensation detectable by a sense of the patient.
30. The system according to anyone of embodiment 1-21 or 41-45, wherein the sensation generator is configured to receive the request from the controller of the implant.
31. The system according to anyone of embodiment 1-21 or 41-45, wherein the request is generated by the controller in response to the sensor measurement having the value outside of the predetermined interval.
32. The system according to anyone of embodiment 1-21 or 41-45, wherein the sensation generator is configured to receive the request from an external controller.
33. The system according to anyone of embodiment 1-21 or 41-45, wherein the generated sensation comprises a plurality of sensation components.
34. The system according to anyone of embodiment 1-21 or 41-45, wherein the sensation generator is configured to create the sensation or sensation components by at least one of: a vibration of the sensation generator;
producing a sound;
providing a photonic signal;
providing a light signal;
providing an electric signal; and
a heat signal.
35. The system according to anyone of embodiment 1-21 or 41-45, further comprising an active unit, communicatively coupled to the processor, for performing controlling or monitoring a bodily function in the patient.
36. The system according to anyone of embodiment 1-21 or 41-45, wherein:
the sensor is configured to measure a physiological parameter of the patient; and the active unit is configured to perform the controlling or monitoring in response to a sensor measurement having a value outside of the predetermined interval, after the processor has been set in the active state.
37. The system according to anyone of embodiment 1-21 or 41-45, wherein a communication unit communicatively coupled to the processor, wherein:
the processor is configured to transmit data relating to the measurement via the communication unit.
38. The system according to anyone of embodiment 1-21 or 41-45, further comprising: a frequency detector, communicatively coupled to the controller and configured to detect a frequency for data communication to or from the communication unit.
39. The system according to anyone of embodiment 1-21 or 41-45, wherein:
the frequency detector comprises an antenna.
40. The system according to anyone of embodiment 1-21 further comprising: the implant according to any of embodiments 17 to 19; and an external controller, adapted to be arranged outside of the patient's body, configured to communicate with the communication unit.
41. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
43. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
44. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part,
wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. A system comprising an implant for implanting in a patient, comprising:
a controller connected to or comprised in the implant, the controller comprising:
a sensor, the sensor being a passive sensor; and
a processor having a sleep mode and an active mode;
wherein:
the sensor is configured to measure a physiological parameter of the patient or a parameter of the implant, and
the controller is further configured to, in response to a sensor measurement having a value outside of a predetermined interval, set the processor in the active mode,
   the system further comprising
a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 316B eHealth Relay Instructions

1. A system for transmitting an instruction from a first external device to an implant, comprising:
an implant implanted in a human patient, the implant comprising an internal control unit configured to control a function of the implant and configured to receive an instruction from an external device; a first external device configured to receive or determine an instruction to be transmitted to the implant, and to transmit the instruction to a second external device; and a second external device configured to receive the instruction transmitted from the first external device, encrypt the instruction, and transmit the encrypted instruction to the implant, wherein the implant is configured to received and decrypt the instruction,
   the system further comprising
   an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system for transmitting an instruction from a first external device to an implant, comprising:
an implant implanted in a human patient, the implant comprising an internal control unit configured to control a function of the implant and configured to receive an instruction from an external device: a first external device configured to receive or determine an instruction to be transmitted to the implant, and to transmit the instruction to a second external device; and a second external device configured to receive the instruction transmitted from the first external device, encrypt the instruction, and transmit the encrypted instruction to the implant, wherein the implant is configured to received and decrypt the instruction,
   the system further comprising
a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an implantable lubrication device comprising:
   a reservoir that stores a lubricating fluid and,
   a fluid connection that introduces the stored lubricating fluid into a damaged joint when the lubrication device is implanted in a patient's body.
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising a male sexual impotence treatment prosthesis apparatus, comprising
   an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
12. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
13. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising a system for treating a female patient to avoid or promote pregnancy comprising a restriction device adapted to postoperatively restrict and release an oviduct of the patient.
18. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to anyone of embodiment 1-21 or 31-35, wherein the second external device is configured to transmit the encrypted instruction by transmitting the encrypted instruction to the first external device, and wherein the first external device is configured to transmit the encrypted instruction to the implant.
23. The system according to anyone of embodiment 1-21 or 31-35, wherein the second external device is configured to transmit the encrypted instruction by transmitting the encrypted instruction to a third external device, and wherein the third external device is configured to transmit the encrypted instruction to the implant.
24. The system according to anyone of embodiment 1-21 or 31-35, wherein the second external device is an encryption device communicatively coupled to the first external device, and wherein any communication between the implant and the second external device is relayed through the first external device.
25. The system according to anyone of embodiment 1-21 or 31-35, wherein the internal control unit is configured to run the decrypted instruction for controlling a function of the implant.
26. The system according to anyone of embodiment 1-21 or 31-35, wherein the first external device is configured to display a user interface for receiving the instruction.
27. The system according to anyone of embodiment 1-21 or 31-35, wherein the implant comprises a set of a predefined program steps, and wherein the implant is configured to verify that the received instruction is comprised in the predefined program steps.
28. The system according to anyone of embodiment 1-21 or 31-35, wherein the implant is configured to reject the instruction in response to the instruction not being comprised in the set of predefined program steps.
29. The system according to anyone of embodiment 1-21 or 31-35, wherein the implant is configured to allow the instruction in response to the instruction being comprised in the set of predefined program steps.
30. The system according to anyone of embodiment 1-21 or 31-35, wherein the first external device and the implant are configured to communicate over a wireless connection.
31. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
32. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
33. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
34. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part,
wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
35. A system for transmitting an instruction from a first external device to an implant, comprising:
an implant implanted in a human patient, the implant comprising an internal control unit configured to control a function of the implant and configured to receive an instruction from an external device;
a first external device configured to receive or determine an instruction to be transmitted to the implant, and to transmit the instruction to a second external device; and
a second external device configured to receive the instruction transmitted from the first external device, encrypt the instruction, and transmit the encrypted instruction to the implant, wherein the implant is configured to received and decrypt the instruction,
   the system further comprising
a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 317B Energy General Microphone

1. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising
   an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising
a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
3. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
   an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
4. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising
a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising an implantable lubrication device comprising:
   a reservoir that stores a lubricating fluid and,
   a fluid connection that introduces the stored lubricating fluid into a damaged joint when the lubrication device is implanted in a patient's body.
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising a device for bone adjustment in a mammal, comprising
   two or more anchoring devices for attaching to a bone in said mammal, an adjustment device for exerting force on said anchoring devices to adjust the distance between or orientation of at least two of said anchoring devices,
wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
8. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising apparatus for drainage of a body fluid or movement of hydraulic treatment fluid in a human or mammal patient comprising
   a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising a male sexual impotence treatment prosthesis apparatus, comprising
   an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
12. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
13. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
15. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
16. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising a system for treating a female patient to avoid or promote pregnancy comprising a restriction device adapted to postoperatively restrict and release an oviduct of the patient.
18. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 30-34, wherein the implantable controller further comprises at least one implantable housing for sealing against fluid, and wherein the computing unit and the microphone are placed inside of the housing.
23. The system according to any one of embodiments 1-21 or 30-34, wherein the computing unit is configured to derive a pulse of the patient from the registered sound related to a bodily function.
24. The system according to any one of embodiments 1-21 or 30-34, wherein the computing unit is configured to derive information related to the patient urinating from the registered sound related to a bodily function.
25. The system according to any one of embodiments 1-21 or 30-34, wherein the computing unit is configured to derive information related to a bowel activity of the patient from the registered sound related to a bodily function.
26. The system according to any one of embodiments 1-21 or 30-34, wherein the computing unit is configured to derive information related to a functional status of the implant from the registered sound related to a function of the implant.
27. The system according to embodiment 26, wherein the computing unit is configured to derive information related to the functional status of an operation device of the implant, from the registered sound related to a function of the implant.
28. The system according to embodiment 27, wherein the computing unit is configured to derive information related to the functional status of at least one of: a motor, a pump and a transmission of the operation device of the implant from, the registered sound related to a function of the implant.
29. The system according to any one of the preceding embodiments or 30-34, further comprising a transceiver, and wherein the controller is configured to transmit a parameter derived from the sound registered by the at least one microphone using the transceiver.
30. A system comprising an implantable controller for controlling an energized implant, when implanted in a patient, the controller comprises:
a computing unit,
at least one microphone, wherein the at least one microphone is configured to register a sound related to at least one of: a bodily function, and a function of the implant,
   the system further comprising an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
31. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
32. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
33. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part,
wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
34. A system comprising an implantable controller for controlling an energized implant, when implanted in a patient, the controller comprises:
a computing unit,
at least one microphone, wherein the at least one microphone is configured to register a sound related to at least one of a bodily function, and a function of the implant,
   the system further comprising
a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 400SE—eHealth_General_System, DDI Inactivation of Remote or Private Key

1. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key.
2. The system according to embodiment 1, wherein the at least one patient remote external device comprises a patient remote external device private key, wherein the DDI via the patient EID external device is able to inactivate the authority and authenticating function of the patient remote external device, thereby inactivating the patient remote external device.
2. The system according to embodiment 1 or 2, wherein said patient EID external device comprises at least one wireless transceiver configured for communication with the DD1 via a first network protocol.
3. The system according to anyone of the preceding embodiments, comprising the DDI, wherein the DDI is adapted to receive command from a HCP EID external device, and to send the received command to the patient EID external device, wherein the DDI comprises a wireless transceiver configured for communication with said patient external device.
4. The system according to anyone of the preceding embodiments, wherein the patient EID external device is adapted to receive the command from the DDI, wherein the command originates from a health care provider, HCP, and wherein the patient EID is adapted to inactivate the patient private key and to send the command to the implanted medical device.
5. The system according to anyone of the preceding embodiments, wherein the patient EID external device is adapted to receive the command from the DDI, wherein the command originates from a health care provider, HCP, wherein the patient EID external device is adapted to receive the command from the HCP via the DDI to inactivate the patient remote external device comprising a patient remote external device private key, and wherein the patient EID external device is further adapted to send this command to the implanted medical device.
6. The system according to anyone of the preceding embodiments, wherein the patient EID external device further comprises at least one wireless transceiver configured for communication with the implanted medical device through a second network protocol.
7. The system according to embodiment any preceding embodiment, wherein at least one of
   the patient private key and a patient remote external device private key comprises a hardware key.
8. The system according to any preceding embodiment, wherein the private key device is at least one of, a smartcard, a key-ring device, a watch an arm or wrist band a neckless or any shaped device.
9. The system according to any preceding embodiment, wherein at least two of:
   the patient remote external device,
   the patient EID external device,
   the patient private key device, and the DDI,
   are configured for wireless communication using a standard network protocol.
10. The system according to any preceding embodiment, wherein at least two of:
   the patient remote external device,
   the patient EID external device,
   the patient private key device, and the DDI,
   are configured for wireless communication using a proprietary network protocol.
11. The system according to any of the preceding embodiments, wherein the patient EID external device is configured to use a first network protocol for communication with the implantable medical device and use a second network protocol for communication with the patient private key device.
12. The system according to any of the preceding embodiments, wherein the patient EID external device is configured to use a first frequency band for communication with the implantable medical device and use a second frequency band for communication with the patient private key device.
13. The system according to any of the preceding embodiments, wherein at least one of the patient remote external device, the patient EID external device, the patient private key device, and the DDI, comprise a Bluetooth transceiver.
14. The system according to any of the preceding embodiments, wherein at least one of the patient remote external device, the patient EID external device, the patient private key device, and the DDI, comprise an UWB transceiver.
15. The system according to embodiment 9, wherein the standard network protocol is one from the list of:
   Radio Frequency type protocol,
   RFID type protocol,
   WLAN type protocol,
   Bluetooth type protocol,
   BLE type protocol,
   NFC type protocol,
   3G/4G/5G type protocol, and
   GSM type protocol.
16. The system according to any of the preceding embodiments, patient EID external device comprises a first wireless transceiver for wireless communication with the implantable medical device, and a second wireless transceiver for wireless communication with the patient private key device, and wherein the second wireless transceiver has longer effective range than the first wireless transceiver.
17. The system according to embodiment 16, wherein the second wireless transceiver has an effective range being one of: 2 times, 4 times, 8 time, 20 times, 50 times or 100 times longer than the effective range of the first wireless transceiver.
18. The system according to embodiments 16 or 17, wherein the second wireless transceiver is configured to be disabled to enable wireless communication using the first wireless transceiver.
19. The system according to any of the preceding embodiments, wherein the patient EID external device is a wearable patient external device or a handset.
20. The system according to any of the preceding embodiments, wherein the data encrypted by the implantable medical device is related to at least one of:
   a battery status,
   a temperature,
   a time, or
   an error.
21. The system according to any of the preceding embodiments, comprising a master private key device configured to allow issuance of new private key device, wherein the HCP or HCP admin have such master private key device adapted to be able to replace and pair a new patient private key device or HCP private key device into the system, through the HCP EID external device.
22. The system according to anyone of the preceding embodiments, wherein the patient remote external device and the patient EID external device are an integrated unit.
23. The system according to anyone of the preceding embodiments, wherein the HCP dedicated device and the HCP EID external device are an integrated unit.
24. The system according to anyone of the preceding embodiments, comprising a measurement device or sensor adapted to deliver a measurement to at least one of the DDI, patent EID external device and a patient display device.
25. The system according to anyone of the preceding aspects, comprising a food sensor, adapted to measure at least if the patient swallows solid food or is drinking fluid, wherein said food sensor is connected to the control unit of a medical device to cause an action to stretch the stomach after a determined amount of food intake.

### Aspect 400 B—eHealth_General_System, DDI Inactivation of Remote or Private Key

1. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device is configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device is configured for treating a patient having a disorder related to the patient's intestine, the implantable medical device comprises
an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
7. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a device for bone adjustment in a mammal, comprising
two or more anchoring devices for attaching to a bone in said mammal, an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a breast implant system comprising a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a system for treating a female patient to avoid or promote pregnancy comprising a restriction device adapted to postoperatively restrict and release an oviduct of the patient.
18. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 47-51, wherein the at least one patient remote external device comprises a patient remote external device private key, wherein the DDI via the patient EID external device is able to inactivate the authority and authenticating function of
the patient remote external device, thereby inactivating the patient remote external device.
23. The system according to any one of embodiments 1-21 or 22 or 47-51, wherein said patient EID external device comprises at least one wireless transceiver configured for communication with the DD1 via a first network protocol.
24. The system according to anyone of the preceding embodiments, comprising the DDI, wherein the DDI is adapted to receive command from a HCP EID external device, and to send the received command to the patient EID external device, wherein the DDI comprises a wireless transceiver configured for communication with said patient external device.
25. The system according to anyone of the preceding embodiments or 47-51, wherein the patient EID external device is adapted to receive the command from the DDI, wherein the command originates from a health care provider, HCP, and wherein the patient EID is adapted to inactivate the patient private key and to send the command to the implanted medical device.
26. The system according to anyone of the preceding embodiments or 47-51, wherein the patient EID external device is adapted to receive the command from the DDI, wherein the command originates from a health care provider, HCP, wherein the patient EID external device is adapted to receive the command from the HCP via the DDI to inactivate the patient remote external device comprising a patient remote external device private key, and wherein the patient EID external device is further adapted to send this command to the implanted medical device.
27. The system according to anyone of the preceding embodiments or 47-51, wherein the patient EID external device further comprises at least one wireless transceiver configured for communication with the implanted medical device through a second network protocol.
28. The system according to embodiment any preceding embodiment or 47-51, wherein at least one of the patient private key and a patient remote external device private key comprises a hardware key.
29. The system according to any preceding embodiment, wherein the private key device is at least one of, a smartcard, a key-ring device, a watch an arm or wrist band a neckless or any shaped device.
30. The system according to any preceding embodiment or 47-51, wherein at least two of:
the patient remote external device,
the patient EID external device,
the patient private key device, and the DDI,
are configured for wireless communication using a standard network protocol.
31. The system according to any preceding embodiment or 47-51, wherein at least two of:
the patient remote external device,
the patient EID external device,
the patient private key device, and the DDI,
are configured for wireless communication using a proprietary network protocol.
32. The system according to any of the preceding embodiments or 47-51, wherein the patient EID external device is configured to use a first network protocol for communication with the implantable medical device and use a second network protocol for communication with the patient private key device.
33. The system according to any of the preceding embodiments or 47-51, wherein the patient EID external device is configured to use a first frequency band for communication with the implantable medical device and use a second frequency band for communication with the patient private key device.
34. The system according to any of the preceding embodiments or 47-51, wherein at least one of the patient remote external device, the patient EID external device, the patient private key device, and the DDI, comprise a Bluetooth transceiver.
35. The system according to any of the preceding embodiments or 47-51, wherein at least one of the patient remote external device, the patient EID external device, the patient private key device, and the DDI, comprise an UWB transceiver.
36. The system according to embodiment 9, wherein the standard network protocol is one from the list of:
Radio Frequency type protocol,
RFID type protocol,
WLAN type protocol,
Bluetooth type protocol,
BLE type protocol,
NFC type protocol,
3G/4G/5G type protocol, and
GSM type protocol.
37. The system according to any of the preceding embodiments or 47-51, patient EID external device comprises a first wireless transceiver for wireless communication with the implantable medical device, and a second wireless transceiver for wireless communication with the patient private key device, and wherein the second wireless transceiver has longer effective range than the first wireless transceiver.
38. The system according to embodiment 16, wherein the second wireless transceiver has an effective range being one of: 2 times, 4 times, 8 time, 20 times, 50 times or 100 times longer than the effective range of the first wireless transceiver.
39. The system according to embodiments 16 or 17, wherein the second wireless transceiver is configured to be disabled to enable wireless communication using the first wireless transceiver.
40. The system according to any of the preceding embodiments or 47-51, wherein the patient EID external device is a wearable patient external device or a handset.
41. The system according to any of the preceding embodiments or 47-51, wherein the data encrypted by the implantable medical device is related to at least one of:
a battery status,
a temperature,
a time, or
an error.
42. The system according to any of the preceding embodiments or 47-51, comprising a master private key device configured to allow issuance of new private key device, wherein the HCP or HCP admin have such master private key device adapted to be able to replace and pair a new patient private key device or HCP private key device into the system, through the HCP EID external device.
43. The system according to anyone of the preceding embodiments or 47-51, wherein the patient remote external device and the patient EID external device are an integrated unit.
44. The system according to anyone of the preceding embodiments or 47-51, wherein the HCP dedicated device and the HCP EID external device are an integrated unit.
45. The system according to anyone of the preceding embodiments or 47-51, comprising a measurement device or sensor adapted to deliver a measurement to at least one of the DDI, patent EID external device and a patient display device.
46. The system according to anyone of the preceding aspects or 47-51, comprising a food sensor, adapted to measure at least if the patient swallows solid food or is drinking fluid, wherein said food sensor is connected to the control unit of a medical device to cause an action to stretch the stomach after a determined amount of food intake.
47. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
48. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
49. A system comprising,
   an implantable medical device adapted to, when implanted in a patient, to communicate with an external device, the external device comprising at least one of a patient remote external device or a patient EID external device, the system further comprising:
   the patient EID external device adapted to communicate with and send commands to the implantable medical device when implanted, to change pre-programmed settings,
   a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
   the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
50. A system comprising,
   an implantable medical device adapted to, when implanted in a patient, to communicate with an external device, the external device comprising at least one of a patient remote external device or a patient EID external device, the system further comprising:
   the patient EID external device adapted to communicate with and send commands to the implantable medical device when implanted, to change pre-programmed settings,
   a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
   the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part,
wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
51. A system comprising,
an implantable medical device adapted to, when implanted in a patient, to communicate with an external device, the external device comprising at least one of a patient remote external device or a patient EID external device, the system further comprising:
the patient EID external device adapted to communicate with and send commands to the implantable medical device when implanted, to change pre-programmed settings,
a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and
a data infrastructure server, DDI, adapted to send commands to the patient EID external device for further transport to the implanted medical device, to inactivate the authority and authenticating function of the patient private key, wherein
the implantable medical device comprises a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 401SE—eHealth_General_Patient_at Hospital

1. A system configured for changing pre-programmed treatment settings in steps of an implantable medical device, when implanted in a patient, by a health care provider, HCP, either in the physical presence of the patient or remotely with the patient on distance, the system comprising:
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device.
2. The system according to anyone of the preceding embodiments, comprising a master private key device that allow issuance of new private key device wherein the HCP or HCP admin have such master private key device adapted to be able to replace and pair a new patient private key device or HCP private key device into the system, through the HCP EID external device.
3. The system according to anyone of the preceding embodiments, wherein the patient remote external device and the patient EID external device are an integrated unit.
4. The system according to anyone of the preceding embodiments, wherein the HCP dedicated device and the HCP EID external device are an integrated unit.
5. The system according to anyone of the preceding embodiments, further comprising a measurement device or sensor adapted to deliver a measurement to at least one of the DDI, patent EID external device and a patient display device.
6. The system according to anyone of the preceding embodiments, further comprising a food sensor adapted to measure at least if the patient swallows solid food or is drinking fluid, wherein said food sensor is configured to be connected to the control unit of a medical device to cause an action to stretch the stomach after a determined amount of food intake.
7. The system according to any preceding embodiment, wherein the HCP EID external device further comprises a wireless transceiver configured for communication with the implanted medical device through a second network protocol.
8. The system according to embodiment any preceding embodiment, wherein the HCP private key device is adapted to be provided to the at least one HCP external device via at least one of;
a reading slot or comparable for the HCP private key device,
a RFID communication, and
a close distance wireless activation communication unit, or electrical direct contact.
9. The system according to anyone of the preceding embodiments, wherein the HCP EID external device comprises at least one of:
reading slot or comparable for the HCP private key device,
a RFID communication, and
a close distance wireless activation communication unit, or electrical direct contact.
10. The system according to any of the preceding embodiments, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
11. The system according to any of the preceding embodiments, wherein the HCP EID external device and the HCP private key device are configured for wireless communication using a standard network protocol.
12. The system according to any of the preceding embodiments, wherein the HCP EID external device and the HCP private key device are configured for wireless communication using a proprietary network protocol.
13. The system according to any of the preceding embodiments, wherein the HCP EID external device is configured to use a first network protocol for communication with the implantable medical device and use a second network protocol for communication with the HCP private key device.
14. The system according to any of the preceding embodiments, wherein the HPC EID external device is configured to use a first frequency band for communication with the implantable medical device and use a second frequency band for communication with the HCP private key device.
15. The system according to any of the preceding embodiments, wherein at least one of the HCP EID external device and the HCP private key device comprises a Bluetooth transceiver.
16. The system according to any of the preceding embodiments, wherein at least one of the HCP EID external device and the HCP private key device comprises a UWB transceiver.

### Aspect 401 B—eHealth_General_Patient_at Hospital

1. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, the implantable medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device is configured for treating a patient having a disorder related to the patient's intestine, the implantable medical device comprises
an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
7. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a device for bone adjustment in a mammal, comprising
two or more anchoring devices for attaching to a bone in said mammal, an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device; wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a breast implant system comprising a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a system for treating a female patient to avoid or promote pregnancy comprising a restriction device adapted to postoperatively restrict and release an oviduct of the patient.
18. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to anyone of the preceding embodiments or 37-41, comprising a master private key device that allow issuance of new private key device wherein the HCP or HCP admin have such master private key device adapted to be able to replace and pair a new patient private key device or HCP private key device into the system, through the HCP EID external device.
23. The system according to anyone of the preceding embodiments or 37-41, wherein the patient remote external device and the patient EID external device are an integrated unit.
24. The system according to anyone of the preceding embodiments or 37-41, wherein the HCP dedicated device and the HCP EID external device are an integrated unit.
25. The system according to anyone of the preceding embodiments or 37-41, further comprising a measurement device or sensor adapted to deliver a measurement to at least one of the DDI, patent EID external device and a patient display device.
26. The system according to anyone of the preceding embodiments or 37-41, further comprising a food sensor adapted to measure at least if the patient swallows solid food or is drinking fluid, wherein said food sensor is configured to be connected to the control unit of a medical device to cause an action to stretch the stomach after a determined amount of food intake.
27. The system according to any preceding embodiment or 37-41, wherein the HCP EID external device further comprises a wireless transceiver configured for communication with the implanted medical device through a second network protocol.
28. The system according to embodiment any preceding embodiment or 37-41, wherein the HCP private key device is adapted to be provided to the at least one HCP external device via at least one of;
   a reading slot or comparable for the HCP private key device,
   a RFID communication, and
   a close distance wireless activation communication unit, or electrical direct contact.
29. The system according to anyone of the preceding embodiments or 37-41, wherein the HCP EID external device comprises at least one of:
   reading slot or comparable for the HCP private key device,
   a RFID communication, and
   a close distance wireless activation communication unit, or electrical direct contact.
30. The system according to any of the preceding embodiments or 37-41, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
31. The system according to any of the preceding embodiments or 37-41, wherein the HCP EID external device and the HCP private key device are configured for wireless communication using a standard network protocol.
32. The system according to any of the preceding embodiments or 37-41, wherein the HCP EID external device and the HCP private key device are configured for wireless communication using a proprietary network protocol.
33. The system according to any of the preceding embodiments or 37-41, wherein the HCP EID external device is configured to use a first network protocol for communication with the implantable medical device and use a second network protocol for communication with the HCP private key device.
34. The system according to any of the preceding embodiments or 37-41, wherein the HPC EID external device is configured to use a first frequency band for communication with the implantable medical device and use a second frequency band for communication with the HCP private key device.
35. The system according to any of the preceding embodiments or 37-41, wherein at least one of the HCP EID external device and the HCP private key device comprises a Bluetooth transceiver.
36. The system according to any of the preceding embodiments or 37-41, wherein at least one of the HCP EID external device and the HCP private key device comprises a UWB transceiver.
37. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
38. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises
a device for treating obesity of a patient, the device comprising
at least one operable stretching device (10; 110) implantable in the patient and adapted to stretch a portion of the patient's stomach wall (12), and
an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
39. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises an obesity treatment device comprising:
at least one operable stretching device (10; 10″; 110a; 110b; 410b; 50; 810) implantable in a patient and adapted to stretch a portion of the patient's stomach wall (12) and
an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
40. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
41. A system configured for changing pre-programmed treatment settings in steps of an implantable medical device, when implanted in a patient, by a health care provider, HCP, either in the physical presence of the patient or remotely with the patient on distance, the system comprising:
an implantable medical device,
at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing:
a HCP private key device comprising a HCP private key, comprising at least one of:
a smart card,
a keyring device,
a watch,
an arm or wrist band,
a necklace, and
any shaped device;
wherein the system further comprises:
a patient private key device comprising a patient private key, comprising at least one of:
a smart card,
a keyring device,
a watch,
an arm or wrist band,
a necklace, and
any shaped device;
wherein both the HCP and patient private key is required for performing said action by the HCP EID external device to change the pre-programmed settings in the implant and to update software of the implantable medical device, when the implantable medical device is implanted, and
wherein the patient private key is adapted to activate, be authenticated, and allowed to perform said command provided by the HCP, either via the HCP EID external device or when the action is performed remotely via a patient EID external device, wherein
the implantable medical device comprises device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 402SE—eHealth_General_EID and DDI

1. A system configured for changing pre-programmed treatment settings in steps of an implantable medical device, when implanted in a patient, by a health care provider, HCP, with the patient on remote on distance, the system comprising:
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device.
2. The system according to embodiment 1, comprising the HCP private key device comprising a HCP private key, comprising at least one of:
a smart card,
a keyring device,
a watch,
an arm or wrist band,
a necklace, and
any shaped device.
3. The system according to anyone of the preceding embodiments, comprising:
the patient private key device comprising a patient private key, comprising at least one of:
a smart card,
a keyring device,
a watch,
an arm or wrist band,
a necklace, and
any shaped device.
4. The system according to anyone of the preceding embodiments, wherein the patient private key is adapted to activate, be authenticated, and allowed to perform said command provided by the HCP, either via the HCP EID external device or when the action is performed remotely via a patient EID external device.
5. The system according to anyone of the preceding embodiments, further comprising a dedicated data infrastructure, DDI, the patient EID external device, and the HCP EID external device, wherein the communication between the patient EID external device and the HCP EID external device is performed via the DDI.
6. The system according to anyone of the preceding embodiments, comprising a master private key device that allows issuance of new private key device wherein the HCP or HCP admin have such master private key device adapted to be able to replace and pair a new patient private key device or HCP private key device into the system.
7. The system according to anyone of the preceding embodiments, wherein the patient remote external device and the patient EID external device are an integrated unit.
8. The system according to anyone of the preceding embodiments, wherein the HCP dedicated device and the HCP EID external device are an integrated unit.
9. The system according to anyone of the preceding embodiments, further comprising a measurement device or sensor adapted to deliver a measurement to at least one of the DDI, patent EID external device and patient display device.
10. The system according to anyone of the preceding embodiments, comprising a food sensor, adapted to measure at least if the patient swallow solid food or is drinking fluid, wherein said food sensor is connected to the control unit of a medical device to cause an action to stretch the stomach after a determined amount of food intake.
11. The system according to any preceding embodiment, wherein the HCP EID external device further comprises a wireless transceiver configured for communication with the implanted medical device through a second network protocol.
12. The system according to embodiment any preceding embodiment, wherein the HCP private key device is adapted to be provided to the at least one HCP external device via at least one of:
   a reading slot or comparable for the HCP private key device,
   a RFID communication, and
   a close distance wireless activation communication unit, or electrical direct contact.
13. The system according to anyone of the preceding embodiments, wherein the HCP EID external device comprises at least one of:
   reading slot or comparable for the HCP private key device,
   a RFID communication, and
   a close distance wireless activation communication unit, or electrical direct contact.
14. The system according to any of the preceding embodiments, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
15. The system according to any of the preceding embodiments, wherein the HCP EID external device and the HCP private key device are configured for wireless communication using a standard network protocol.
16. The system according to any of the preceding embodiments, wherein the HCP EID external device and the HCP private key device are configured for wireless communication using a proprietary network protocol.
17. The system according to any of the preceding embodiments, wherein the HCP EID external device is configured to use a first network protocol for communication with the implantable medical device and use a second network protocol for communication with the HCP private key device.
18. The system according to any of the preceding embodiments, wherein the HPC EID external device is configured to use a first frequency band for communication with the implantable medical device and use a second frequency band for communication with the HCP private key device.
19. The system according to any of the preceding embodiments, wherein at least one of the HCP EID external device and the HCP private key device comprises a Bluetooth transceiver.
20. The system according to any of the preceding embodiments, wherein at least one of the HCP EID external device and the HCP private key device comprises a UWB transceiver.

### Aspect 402B—eHealth_General_EID and DDI

1. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
the implantable medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion,
wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
2. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device is configured for treating a patient having a disorder related to the patient's intestine, the implantable medical device comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises a drug delivery device for injecting a drug into a patient's body such as stimulation of penis erection wherein
the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable lubrication device comprising:
   a reservoir that stores a lubricating fluid and,
   a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
7. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises a device for bone adjustment in a mammal, comprising
two or more anchoring devices for attaching to a bone in said mammal, an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device a device for treatment or monitoring of an aneurysm, comprising
an implantable member adapted to hold fluid, the member being adapted to be placed in connection with a blood vessel having the aneurysm and to exercise a pressure on the aneurysm,
   a control unit adapted to control pressure adjustments of the implantable member.
13. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
14. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises a breast implant system comprising a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises a system for treating a female patient to avoid or promote pregnancy comprising a restriction device adapted to postoperatively restrict and release an oviduct of the patient.
18. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 41-45, comprising the HCP private key device comprising a HCP private key, comprising at least one of:
a smart card,
a keyring device,
a watch,
an arm or wrist band,
a necklace, and
any shaped device.
23. The system according to anyone of the preceding embodiments or 41-45, comprising:
the patient private key device comprising a patient private key, comprising at least one of:
a smart card,
a keyring device,
a watch,
an arm or wrist band,
a necklace, and
any shaped device.
24. The system according to anyone of the preceding embodiments or 41-45, wherein the patient private key is adapted to activate, be authenticated, and allowed to perform said command provided by the HCP, either via the HCP EID external device or when the action is performed remotely via a patient EID external device.
25. The system according to anyone of the preceding embodiments or 41-45, further comprising a dedicated data infrastructure, DDI, the patient EID external device, and the HCP EID external device, wherein the communication between the patient EID external device and the HCP EID external device is performed via the DDI.
26. The system according to anyone of the preceding embodiments or 41-45, comprising a master private key device that allows issuance of new private key device wherein the HCP or HCP admin have such master private key device adapted to be able to replace and pair a new patient private key device or HCP private key device into the system.
27. The system according to anyone of the preceding embodiments or 41-45, wherein the patient remote external device and the patient EID external device are an integrated unit.
28. The system according to anyone of the preceding embodiments or 41-45, wherein the HCP dedicated device and the HCP EID external device are an integrated unit.
29. The system according to anyone of the preceding embodiments or 41-45, further comprising a measurement device or sensor adapted to deliver a measurement to at least one of the DDI, patent EID external device and patient display device.
30. The system according to anyone of the preceding embodiments or 41-45, comprising a food sensor, adapted to measure at least if the patient swallow solid food or is drinking fluid, wherein said food sensor is connected to the control unit of a medical device to cause an action to stretch the stomach after a determined amount of food intake.
31. The system according to any preceding embodiment or 41-45, wherein the HCP EID external device further comprises a wireless transceiver configured for communication with the implanted medical device through a second network protocol.
32. The system according to embodiment any preceding embodiment or 41-45, wherein the HCP private key device is adapted to be provided to the at least one HCP external device via at least one of:
   a reading slot or comparable for the HCP private key device,
   a RFID communication, and
   a close distance wireless activation communication unit, or electrical direct contact.
33. The system according to anyone of the preceding embodiments or 41-45, wherein the HCP EID external device comprises at least one of:
   reading slot or comparable for the HCP private key device,
   a RFID communication, and
   a close distance wireless activation communication unit, or electrical direct contact.
34. The system according to any of the preceding embodiments or 41-45, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
35. The system according to any of the preceding embodiments or 41-45, wherein the HCP EID external device and the HCP private key device are configured for wireless communication using a standard network protocol.
36. The system according to any of the preceding embodiments or 41-45, wherein the HCP EID external device and the HCP private key device are configured for wireless communication using a proprietary network protocol.
37. The system according to any of the preceding embodiments or 41-45, wherein the HCP EID external device is configured to use a first network protocol for communication with the implantable medical device and use a second network protocol for communication with the HCP private key device.
38. The system according to any of the preceding embodiments or 41-45, wherein the HPC EID external device is configured to use a first frequency band for communication with the implantable medical device and use a second frequency band for communication with the HCP private key device.
39. The system according to any of the preceding embodiments or 41-45, wherein at least one of the HCP EID external device and the HCP private key device comprises a Bluetooth transceiver.
40. The system according to any of the preceding embodiments or 41-45, wherein at least one of the HCP EID external device and the HCP private key device comprises a UWB transceiver.
41. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
42. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
wherein the stretching device comprises a first and a second engaging part (10″; 110a; 110b; 210a; 210b), wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
43. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises: an obesity treatment device comprising:
at least one operable stretching device (10; 10″; 110a; 110b; 410b; 50; 810) implantable in a patient and adapted to stretch a portion of the patient's stomach wall (12) and
an implantable operation device (90; 42; 54; 40; 217; 218; 452) for operating the stretching device, when implanted, to stretch the stomach wall portion such that satiety is created,
wherein the operable stretching device is adapted to be placed against the outside of the stomach wall.
44 A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
45. A system configured for changing pre-programmed treatment settings in steps of an implantable medical device, when implanted in a patient, by a health care provider, HCP, with the patient on remote on distance, the system comprising:
an implantable medical device;
at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP;
wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 403SE—eHealth_General

1. A system configured for changing pre-programmed treatment settings of an implantable medical device, when implanted in a patient, from a distant remote location in relation to the patient, the system comprising:
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.
2. The system according to embodiment 1, wherein at least one of the patient private key device or HCP private key device comprises a hardware key.
3. The system according to embodiment 1 or 2, wherein the private key device is at least one of, a smartcard, a key-ring device, a watch an arm or wrist band a neckless or any shaped device.
4. The system according to anyone of the preceding embodiments, comprising a master private key device that allow issuance of new private key device wherein the HCP or HCP admin have such master private key device adapted to be able to replace and pair a new patient private key device or HCP private key device into the system, through the HCP EID external device.
5. The system according to anyone of the preceding embodiments, wherein the patient remote external device and the patient EID external device is an integrated unit.
6. The system according to anyone of the preceding embodiments, wherein the HCP dedicated device and the HCP EID external device are an integrated unit.
7. The system according to anyone of the preceding embodiments, comprising a measurement device or sensor adapted to deliver a measurement to at least one of the DDI, patent EID external device and a patient display device.
8. The system according to anyone of the preceding embodiments, comprising a food sensor, adapted to measure at least if the patient swallow solid food or is drinking fluid, wherein said food sensor is connected to the control unit of a medical device to cause an action to stretch the stomach after a determined amount of food intake.
9. The system according to any preceding embodiment, wherein the HCP EID external device further comprises a wireless transceiver configured for communication with the implanted medical device through a second network protocol.
10. The system according to embodiment any preceding embodiment, wherein the HCP private key device is adapted to be provided to the at least one HCP external device via at least one of;
a reading slot or comparable for the HCP private key device,
a RFID communication, and
a close distance wireless activation communication unit, or electrical direct contact.
11. The system according to anyone of the preceding embodiments, wherein the HCP EID external device comprises at least one of:
reading slot or comparable for the HCP private key device,
a RFID communication, and
a close distance wireless activation communication unit, or electrical direct contact.
12. The system according to any of the preceding embodiments, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
13. The system according to any of the preceding embodiments, wherein the HCP EID external device and the HCP private key device are configured for wireless communication using a standard network protocol.
14. The system according to any of the preceding embodiments, wherein the HCP EID external device and the HCP private key device are configured for wireless communication using a proprietary network protocol.
15. The system according to any of the preceding embodiments, wherein the HCP EID external device is configured to use a first network protocol for communication with the implantable medical device and use a second network protocol for communication with the HCP private key device.
16. The system according to any of the preceding embodiments, wherein the HPC EID external device is configured to use a first frequency band for communication with the implantable medical device and use a second frequency band for communication with the HCP private key device.
17. The system according to any of the preceding embodiments, wherein at least one of the HCP EID external device and the HCP private key device comprises a Bluetooth transceiver.
18. The system according to any of the preceding embodiments, wherein at least one of the HCP EID external device and the HCP private key device comprises a UWB transceiver.

### Aspect 403 B—eHealth_General

1. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, the implantable medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
   a reading slot or comparable for the HCP private key device,
   a RFID communication, and
   other close distance wireless activation communication or electrical direct contact,
wherein the HCP EID external device further comprising at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device is configured for treating a patient having a disorder related to the patient's intestine, the implantable medical device comprises
   an artificial intestine section adapted to being implanted inside a patient's body, along with an accumulator for accumulating energy, the artificial intestine section has a first open end portion and a second open end portion in flow communication with one another, wherein at least the first open end is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable lubrication device comprising:
a reservoir that stores a lubricating fluid and,
a fluid connection that introduces the stored lubricating fluid into a damaged joint when the lubrication device is implanted in a patient's body, wherein the lubricating device is configured to be completely implanted into the patient's body such that a damaged joint can post-operatively be lubricated from within the patient's body, and wherein
the operative supply of lubricating fluid to the damaged joint is controlled continuously, intermittently, periodically or depending on a physical parameter of the patient, such as a fluid level within the joint.
7. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a breast implant system comprising a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a system for treating a female patient to avoid or promote pregnancy comprising a restriction device adapted to postoperatively restrict and release an oviduct of the patient.
18. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an apparatus for controlling a flow of sperms in a uterine tube of a female patient, the apparatus comprising: an implantable constriction device for constricting at least one portion of the uterine tube wall to at least partly constrict the uterine tube to influence the flow of sperms in the uterine tube.
19. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. The system according to any one of embodiments 1-21 or 39-43, wherein at least one of
the patient private key device or HCP private key device comprises a hardware key.
23. The system according to any one of the preceding embodiments or 39-43, or 41 wherein the private key device is at least one of, a smartcard, a key-ring device, a watch an arm or wrist band a neckless or any shaped device.
24. The system according to anyone of the preceding embodiments or 39-43, comprising a master private key device that allow issuance of new private key device wherein the HCP or HCP admin have such master private key device adapted to be able to replace and pair a new patient private key device or HCP private key device into the system, through the HCP EID external device.
25. The system according to anyone of the preceding embodiments or 39-43, wherein the patient remote external device and the patient EID external device is an integrated unit.
26. The system according to anyone of the preceding embodiments or 39-43, wherein the HCP dedicated device and the HCP EID external device are an integrated unit.
27. The system according to anyone of the preceding embodiments or 39-43, comprising a measurement device or sensor adapted to deliver a measurement to at least one of the DDI, patent EID external device and a patient display device.
28. The system according to anyone of the preceding embodiments or 39-43, comprising a food sensor, adapted to measure at least if the patient swallow solid food or is drinking fluid, wherein said food sensor is connected to the control unit of a medical device to cause an action to stretch the stomach after a determined amount of food intake.
29. The system according to any preceding embodiment or 39-43, wherein the HCP EID external device further comprises a wireless transceiver configured for communication with the implanted medical device through a second network protocol.
30. The system according to embodiment any preceding embodiment or 39-43, wherein the HCP private key device is adapted to be provided to the at least one HCP external device via at least one of;
a reading slot or comparable for the HCP private key device,
a RFID communication, and
a close distance wireless activation communication unit, or electrical direct contact.
31. The system according to anyone of the preceding embodiments or 39-43, wherein the HCP EID external device comprises at least one of:
reading slot or comparable for the HCP private key device,
a RFID communication, and
a close distance wireless activation communication unit, or electrical direct contact.
32. The system according to any of the preceding embodiments or 39-43, wherein the HCP EID external device is adapted to receive a command from an HCP dedicated device to change said pre-programmed treatment steps of the implantable medical device, when implanted, wherein the HCP dedicated device is further adapted to be activated, authenticated, and allowed to perform said command by the HCP providing their private key.
33. The system according to any of the preceding embodiments or 39-43, wherein the HCP EID external device and the HCP private key device are configured for wireless communication using a standard network protocol.
34. The system according to any of the preceding embodiments or 39-43, wherein the HCP EID external device and the HCP private key device are configured for wireless communication using a proprietary network protocol.
35. The system according to any of the preceding embodiments or 39-43, wherein the HCP EID external device is configured to use a first network protocol for communication with the implantable medical device and use a second network protocol for communication with the HCP private key device.
36. The system according to any of the preceding embodiments or 39-43, wherein the HPC EID external device is configured to use a first frequency band for communication with the implantable medical device and use a second frequency band for communication with the HCP private key device.
37. The system according to any of the preceding embodiments or 39-43, wherein at least one of the HCP EID external device and the HCP private key device comprises a Bluetooth transceiver.
38. The system according to any of the preceding embodiments or 39-43, wherein at least one of the HCP EID external device and the HCP private key device comprises a UWB transceiver.
39. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device, wherein
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
40. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises:
a device for treating obesity of a patient, the device comprising
   at least one operable stretching device (10; 110) implantable in the patient and adapted to stretch a portion of the patient's stomach wall (12), and
   an implantable control unit (42) for automatically controlling the operable stretching device, when the control unit and stretching device are implanted, to stretch the stomach wall portion in connection with the patient eating such that satiety is created,
   wherein the stretching device comprises a first and a second engaging part (10″; 110a; 110b; 210a; 210b), wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
41. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises an obesity treatment device comprising:
   at least one operable stretching device (10; 10″; 110a; 110b; 410b; 50; 810) implantable in a patient and adapted to stretch a portion of the patient's stomach wall (12) and
   an implantable operation device (90; 42; 54; 40; 217; 218; 452) for operating the stretching device, when implanted, to stretch the stomach wall portion such that satiety is created,
   wherein the operable stretching device is adapted to be placed against the outside of the stomach wall.
42 A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
43. A system configured for changing pre-programmed treatment settings of an implantable medical device, when implanted in a patient, from a distant remote location in relation to the patient, the system comprising:
an implantable medical device;
at least one health care provider, HCP, external device adapted to receive a command from the HCP to change said pre-programmed treatment settings of an implanted medical device, further adapted to be activated and authenticated and allowed to perform said command by the HCP providing,
a HCP private key device adapted to be provided to an HCP EID external device via at least one of; a reading slot or comparable for the HCP private key device, a RFID communication or other close distance wireless activation communication,
wherein the HCP EID external device comprising at least one of;
a reading slot or comparable for the HCP private key device,
a RFID communication, and
other close distance wireless activation communication or electrical direct contact,
wherein the HCP EID external device further comprising at least one wireless transceiver configured for communication with a patient EID external device, through a first network protocol,
wherein the system comprises the patient EID external device, the patient EID external device being adapted to receive command from said HCP external device, and to relay the received command without modifying said command to the implanted medical device,
wherein the patient EID external device comprising one wireless transceiver configured for communication with said patient external device,
wherein the patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key, wherein
the implantable medical device comprises
a device for treating obesity of a patient, the device comprising:
a. at least one operable stretching device implantable in the patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 447B Data_Packet_Encryption—External Device

1. An external system for providing remote instructions to an implantable medical device, the external system being configured to:
   provide instructions to be transmitted to the implantable medical device,
   derive a checksum from the instructions,
   electronically sign the instructions and the checksum,
   form a data packet from the instructions, the electronic signature and the checksum, wherein the external system comprises a wireless transmitter configured to wirelessly send the data packet to the implantable medical device.
2. The external system according to claim 1, wherein the external system is further configured to encrypt the data packet at the external system.
3. The external system according to any one of claims 1 and 2, wherein the wireless transmitter is part of a wireless transceiver comprised in the external system.
4. The external system according to any one of the preceding claims, wherein the external system comprises a first external device and a second external device, and wherein the first external device is configured to transmit the data packet to the second external device, and wherein the second external device is configured to transmit the data packet wirelessly to the implantable medical device without changing the data packet.
5. The external system according to any one of the preceding claims, wherein the external system comprises a first external device and a second external device, and wherein the first external device is configured to transmit the data packet to the second external device, and wherein the second external device is configured to transmit the data packet wirelessly to the implantable medical device without full decryption of the data packet.
6. The external system according to any one of the preceding claims, wherein the external system is configured to transmit at least one instruction for altering the control program of the implantable medical device, to the implantable medical device.
7. The external system according to any one of the preceding claims, wherein the external system is configured to provide at least one instruction to the implantable medical device for altering at least one parameter for affecting the control of the implantable medical device.
8. The external system according to claim 7, wherein the external system is configured to provide at least one instruction for updating at least one parameter of the control program to a parameter value comprised in a set of parameter values stored in the implantable medical device.
9. The external system according to any one of the preceding claims, wherein the first external device is configured to send the data packet from the first external device to the second external device using a first network protocol and send the data packet from the second external device to the implantable medical device using a second network protocol.
10. The external system according to any one of the preceding claims, wherein the first external device is configured to send the data packet from the first external device to the second external device using wired communication and send the data packet from the second external device to the implantable medical device using wireless communication.
11. The external system according to any one of claims 1-9, wherein the first external device is configured to wirelessly send the data packet from the first external device to the second external device using a first network protocol, and wirelessly send the data packet from the second external device to the implantable medical device using a second network protocol.
12. The external system according to any one of claims 1-9 or 11, wherein the first external device is configured to wirelessly send the data packet from the first external device to the second external device using a first frequency band, and wirelessly send the data packet from the second external device to the implantable medical device using a second frequency band.
13. The external system according to any one of the preceding claims, wherein the first external device is configured to wirelessly send the data packet from the first external device to the second external device using a first wireless technology, and wirelessly send the data packet from the second external device to the implantable medical device using a second wireless technology.
14. The external system according to any one of the preceding claims, wherein the external system is configured to electronically sign the instructions at the external system using a key of the external system.
15. The external system according to claim 14, wherein the key is a non-extractable key.
16. The external system according to any one of claims 14 and 15, wherein the second external device is configured to perform a proof of possession operation comprising the steps of:
   transmitting, form the first external device to the second external device, a query based on a public key associated with the private of the external system,
   receiving, at the second external device, a response based on the possession of the private key in the first external device, and
   verifying that the response based on the possession of the private key matches the query based on a public key.
17. The external system according to any one of the preceding claims, wherein:
   the first external device is configured to form the data packet and electronically sign the instruction using a first private key, and
   the second external device is configured to:
      receive the data packet from the first external device,
      verify that the first external device is a trusted transmitter,
      in response to the verification, electronically sign the data packet using
a second private key, and
   transmit the data packet from the second external device to the implantable medical device.
18. The external system according to any one of the preceding claims, wherein the checksum is configured to verify that no changes have been made to the bit stream forming the instructions.
19. The external system according to any one of the preceding claims, wherein the first external device is configured to at least one of: electronically sign the instructions and encrypt the data packet using a key placed on a key device external to the first external device.
20. The external system according to any one of the preceding claims, wherein the external system further comprises a key device configured to hold at least one private key.
21. The external system according to claim 20, wherein the key device comprises a wireless transmitter for wirelessly transmitting the at least one private key or a signal based on the private key, to the first external device.
22. The external system according to any one of the preceding claims, wherein the second external device is configured to at least one of: electronically sign the instructions and encrypt the data packet using a key placed on a key device external to the second external device.
23. The external system according to any one of claims 14-22, wherein the external system further comprises a second key device configured to hold at least one second private key.
24. The external system according to claim 23, wherein the second key device comprises a wireless transmitter for wirelessly transmitting the at least one private key or a signal based on the private key to the second external device.
25. The external system according to claim 14, further comprising a second key device comprising a wireless transmitter for wirelessly transmitting at least one second private key or a signal based on the second private key to the first external device.
26. The external system according to any one of claims 14-25, wherein at least one of the key device and the second key device comprises at least one of: a key card, a wearable device and a handset.
27. The external system according to any one of the preceding claims, wherein the first external device is configured to be unlocked by user credentials provided to the first external device.
28. The external system according to claim 27, wherein the first external device is configured to be unlocked by user credentials comprising a username and a password.
29. The external system according to claim 28, wherein the first external device is configured to be unlocked by user credentials comprising a PIN-code.
30. The external system according to any one of claims 27-29, wherein the first external device is configured to verify the user credentials by comparing the user credentials with user credentials stored in the first external device.
31. The external system according to claim 30, wherein the first external device is configured to verify the user credentials by comparing the user credentials with user credentials stored in the first external device by the manufacturer of the first external device.
32. The external system according to any one of claims 27-31, wherein the first external device is configured verify the user credentials by comparing the user credentials with user credentials stored as hardware or software in the first external device.
33. The external system according to any one of claims 27-32, wherein the first external device is configured verify the user credentials by communicating with a remote server.
34. The external system according to any one of the preceding claims, wherein the second external device is configured to be unlocked by user credentials provided to the second external device.
35. The external system according to claim 34, wherein the first external device is configured to be unlocked by user credentials comprising a username and a password.
36. The external system according to claim 34, wherein the first external device is configured to be unlocked by user credentials comprising a PIN-code.
37. The external system according to claim 36, wherein the second external device is configured to verify the user credentials by comparing the user credentials with user credentials stored in the second external device.
38. The external system according to claim 37, wherein the second external device is configured to verify the user credentials by comparing the user credentials with user credentials stored in the second external device by the manufacturer of the second external device.
39. The external system according to any one of claims 37 and 38, wherein the second external device is configured verify the user credentials by comparing the user credentials with user credentials stored as hardware or software in the second external device.
40. The external system according to any one of claims 37-39, wherein the second external device is configured verify the user credentials by communicating with a remote server.
41. The external system according to any one of the preceding claims, wherein the external system is configured to function without connection to the Internet.
42. The external system according to any one of the preceding claims, wherein the external system is configured to communicate with the implantable medical device independently of time.
43. The external system according to any one of the preceding claims 14-42, wherein the first and second private keys are different.
44. The external system according to claim 43, wherein the first and second private keys comprises at least one common element.
45. The external system according to any one of claims 14-44, wherein at least one first and second external device are configured to be unlocked by at least one of the first and second private key.
46. The external system according to any one of the preceding claims, wherein the external system comprises a central server, and wherein the central server is configured to form a data packet from the instructions, the electronic signature and the checksum and further configured to provide the formed data packet to the first external device.
47. The external system according to claim 46, wherein the central server can be accessed by at least one healthcare professional, such that the healthcare professional can provide input to the central server for forming the instructions to be sent to the implantable medical device.
48. The external system according to claim 46, wherein the central server can be accessed by at least one patient, such that the patient can provide input to the central server for verifying at least one of: the authenticity of the healthcare professional and the correctness of the instructions.
49. The external system according to claim 48, wherein the healthcare provider can electronically sign the instructions at the central server.
50. The external system according to any one of claims 48 and 49, wherein the patient can electronically sign the instructions at the central server.
51. The external system according to any one of claims 46-50, wherein the central server is configured to verify the authenticity of the first and second key and electronically sign the instructions using the first and second key.
52. The external system according to any one of the preceding claims, wherein the second key is a user key, and wherein the external system is configured to use the second key for at least one of:
   approving that communication is transmitted to the implantable medical device, and
   approving that a healthcare provider prepares an instruction to the implantable medical device.
53. The external system according to claim 52, wherein the approval step can be performed by first or second external device.
54. The external system according to any one of claims 14-53, wherein the first key is required to create an instruction to the implantable medical device and the second key is required to transmit the created instruction to the implantable medical device.
55. The external system according to any one of claims 2-54, wherein at least one of the first and second external device comprises an input button configured to be used for verifying user presence.
56. The external system according to claim 55, wherein the input button con be configured to replace at least one of:
   input of at least one key to at least one of the first and second external device, and
   input of credentials into at least one of the first and second external device.
56. The external system according to claim 55, wherein the input button is configured to replace the second key.
57. The external system according to any one of the preceding claims, wherein the external system is configured to transmit the data packet to the implantable medical device, and wherein the data packet comprises:
   at least one instruction signed by a first key and
   a public key including information about which root have created the public key.
58. The external system according to any one of claims 2-57, wherein at least one of the first and second external device is configured to enable communication with the implantable medical device based on at least one password being provided to at least one of the first and second external device.
59. The external system according to claim 58, wherein at least one of the first and second external device is configured to enable communication with the implantable medical device based on two passwords being provided to at least one of the first and second external device.
60. The external system according to claim 59, wherein at least one of the first and second external device is configured to enable communication with the implantable medical device based on one patient password and one healthcare provider passwords being provided to at least one of the first and second external device.
61. The external system according to any one of the preceding claims, wherein at least one of the first and second external devices are configured to perform a verification query operation with at least one of the first and second key device, the verification query operation comprising:
   transmitting, from the first or second external devices, a query comprising a computational challenge to at least one of the first and second key device,
   receiving, at the first or second external devices, a response based on the transmitted computational challenge, and
   verifying, at the first or second external devices, the received response.
62. The external system according to claim 61, wherein at least one of the first and second external devices are configured to perform a verification query operation in the form of a proof of possession operation comprising:
   receiving a public key of at least one of the first and second key devices, the public key being associated with a private key of the first or second key device,
   transmitting, from at least one of the first and second external devices, a computational challenge to the first or second key device, based on the public key received from the first or second key device,
   receiving a response from the first or second key device based on the possession of the private key in the first or second key device, and
   verifying that the response based on the possession of the private key matches the query based on a public key.
63. A medical system comprising the external system according to any one of the preceding claims and an implantable medical device.
64. The medical system according to claim 63, wherein the implantable medical device comprises an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion,
wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
65. The medical system according to claim 63, wherein the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
66. The medical system according to claim 63, wherein the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
67. The medical system according to claim 63, wherein the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
68. The medical system according to claim 63, wherein the implantable medical device comprises a drug delivery device for injecting a drug into a patient's body such as stimulation of penis erection wherein
   the drug is injected into the patient's body into at least one of
   both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
69. The medical system according to claim 63, wherein the implantable medical device comprises an implantable lubrication device comprising:
   a reservoir that stores a lubricating fluid and,
   a fluid connection that introduces the stored lubricating fluid into a damaged joint when the lubrication device is implanted in a patient's body.
   wherein the lubricating device is configured to be completely implanted into the patient's body such that a damaged joint can post-operatively be lubricated from within the patient's body,
   and wherein the operative supply of lubricating fluid to the damaged joint is controlled continuously, intermittently, periodically or depending on a physical parameter of the patient, such as a fluid level within the joint.
70. The medical system according to claim 63, wherein the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
71. The medical system according to claim 63, wherein the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
72. The medical system according to claim 63, wherein the implantable medical device comprises a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
73. The medical system according to claim 63, wherein the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
74. The medical system according to claim 63, wherein the implantable medical device comprises a male sexual impotence treatment prosthesis apparatus, comprising
an operable prosthesis implantable in the cavities of the corpora cavernosa of an impotent patient to provide erect penile condition, when the prosthesis is operated, wherein an energy transmission device for wireless transmission of energy from outside the patient's body to inside the patient's body for use in connection with the operation of the prosthesis, when the prosthesis is implanted.
75. The medical system according to claim 63, wherein the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
76. The medical system according to claim 63, wherein the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
77. The medical system according to claim 63, wherein the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
78. The medical system according to claim 63, wherein the implantable medical device comprises a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
79. The medical system according to claim 63, wherein the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
80. The medical system according to claim 63, wherein the implantable medical device comprises a system for treating a female patient to avoid or promote pregnancy comprising a restriction device adapted to postoperatively restrict and release an oviduct of the patient.
81. The medical system according to claim 63, wherein the implantable medical device comprises an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
82. The medical system according to claim 63, wherein the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
83. The medical system according to claim 63, wherein the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
84. The medical system according to claim 63, wherein the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
85. The medical system according to claim 63, wherein the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
86. The medical system according to claim 63, wherein the implantable medical device comprises a device for treating obesity of a patient, the device comprising:
   at least one operable stretching device implantable in the patient and adapted to stretch a portion of the patient's stomach wall, and
   an implantable control unit for automatically controlling the operable stretching device, when the control unit and stretching device are implanted, to stretch the stomach wall portion in connection with the patient eating such that satiety is created,
   wherein the stretching device comprises a first and a second engaging part, wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
87. The medical system according to claim 63, wherein the implantable medical device comprises an obesity treatment device comprising:
   at least one operable stretching device implantable in a patient and adapted to stretch a portion of the patient's stomach wall and
   an implantable operation device for operating the stretching device, when implanted, to stretch the stomach wall portion such that satiety is created,
   wherein the operable stretching device is adapted to be placed against the outside of the stomach wall.
88. The medical system according to claim 63, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
89. The medical system according to claim 63, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 447BB Data_Packet_Encryption—External Device

1. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the
   checksum, wherein the external system comprises a wireless transmitter configured to
   wirelessly send the data packet to the implantable medical device,
wherein the implantable medical device comprises:
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion,
wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
2. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: a system for treating a female patient to avoid or promote pregnancy comprising a restriction device adapted to postoperatively restrict and release an oviduct of the patient.
18. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the Joint cavity for lubricating the synovial Joint.
20. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises: an obesity treatment device comprising:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. An external system for providing remote instructions to an implantable medical device, the external system being configured to:
   provide instructions to be transmitted to the implantable medical device,
      derive a checksum from the instructions,
      electronically sign the instructions and the checksum,
      form a data packet from the instructions, the electronic signature and the checksum, wherein the external system comprises a wireless transmitter configured to wirelessly send the data packet to the implantable medical device,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 447A Data_Packet_Encryption—Implant

1. An implantable medical device configured to receive remote instructions from an external system, the implantable medical device comprising:
   a wireless receiver configured to receive wirelessly transmitted data packets from the external system,
   a computing unit configured to:
      verify the electronic signature, and use a checksum provided in the data packet to verify the integrity of the
         instructions.
2. The implantable medical device according to claim 1, wherein the computing unit is configured to decrypt the data packet.
3. The implantable medical device according to any one of claims 1 and 2, wherein the computing unit is configured to use the checksum to verify that the bit stream making up the instructions is unchanged.
4. The implantable medical device according to any one of claims 1 and 2, wherein the wireless receiver is part of a wireless transceiver.
5. The implantable medical device according to any one of claims 1-4, wherein the computing unit comprises a memory unit configured to store electronic signatures, and wherein the computing unit is configured to verify the electronic signature my comparing the electronic signature with the electronic signatures stored in the memory unit.
6. The implantable medical device according to any one of the preceding claims, wherein the implantable medical device comprises a control program configured to control at least one function of
the implantable medical device, and wherein computing unit is configured to alter the control program on the basis of the received instructions.
7. The implantable medical device according to any one of the preceding claims, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
8. The implantable medical device according to claim 7, wherein the computing unit comprises a memory unit configured to store parameter values, and wherein the method further comprises the step of verifying that the instructions for altering the at least one parameter will result in the at least one parameter being updated to a parameter value comprised in the set of stored parameter values.
9. The implantable medical device according to any one of the preceding claims, wherein the implantable medical device comprises a central unit, comprising at least one of a wireless receiver and a wireless transceiver, and a security module connected to the central unit, wherein the implantable medical device is configured to transfer the data packet from the central unit to the security module and wherein the security module is configured to performing at least a portion of at least one of the decryption and the signature verification.
10. The implantable medical device according to claim 9, wherein the security module comprises a set of rules for accepting communication from the central unit, and wherein the security module is configured to verify compliance with the set of rules.
11. The implantable medical device according to claim 10, wherein wireless receiver or wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
12. The implantable medical device according to any one of the preceding claims, wherein the implantable medical device is configured to at least one of decrypting the data packet and verifying the electronic signature using a private key of the implantable medical device.
13. The implantable medical device according to any one of claims 10-12, wherein the private key is a non-extractable key.
14. The implantable medical device according to any one of claims 10-13, wherein the implantable medical device is configured to perform a proof of possession operation comprising: transmitting, from the implantable medical device to the external system, a query based on a public key associated with the private key of the external system, receiving, at the implantable medical device, a response based on the possession of the private key in the external system, and verifying that the response based on the possession of the private key matches the query based on a public key.
15. The implantable medical device according to any one of the preceding claims, wherein the implantable medical device is configured to communicate with the external system independently of time.
16. The implantable medical device according to any one of the preceding claims, wherein the private key is provided in the implantable medical device by the manufacturer of the implantable medical device.
17. The implantable medical device according to claim 16, wherein the private key is stored as hardware or software in the implantable medical device.
18. The implantable medical device according to any one of the preceding claims 12-17, wherein the implantable medical device is configured to:
   verify a first electronic signature made using at least one of a first key and a second key, and
   verifying a second electronic signature made using at least one of a first key and a second key.
19. The implantable medical device according to claim 18, wherein at least one of the first and second keys is a private key.
20. The implantable medical device according to claim 18, wherein the first and second keys are different.
21. The implantable medical device according to claim 20, wherein the first and second keys comprises at least one common element.
22. The implantable medical device according to any one of claims 18-21, wherein the implantable medical device is configured to:
   verify a first electronic signature to allow communication from the external system to the implantable medical device, and
   verify a second electronic signature to allow an instruction received in the communication to alter the control program running on the implantable medical device.
23. The implantable medical device according to claim 22, wherein the first electronic signature is an electronic signature linked to the user of the implantable medical device and the second electronic signature is an electronic signature linked to a healthcare provider.
24. The implantable medical device according to any one of claims 12-23, wherein only a portion of the private key is needed to at least one of: decrypt the data packet and verify the electronic signature.
25. The implantable medical device according to any one of claims 12-23, wherein the implantable medical device trusts any external device holding the private key.
26. The implantable medical device according to any one of the preceding claims, wherein the implantable medical device is configured to receive the data packet comprising:
   at least one instruction signed by a private key of the external system, and
   a public key including information about which root have created the public key.
27. The implantable medical device according to any one of the preceding claims, wherein the implantable medical device is configured to accept communication from an external system based on at least one password being provided to the implantable medical device.
28. The implantable medical device according to claim 27, wherein the implantable medical device is configured to accept communication from an external system based on two passwords being provided to the implantable medical device.
29. The implantable medical device according to claim 28, wherein the implantable medical device is configured to accept communication from an external system based on one patient password and one healthcare provider passwords being provided to the implantable medical device.
30. The implantable medical device according to any one of the preceding claims, wherein the implantable medical device comprises an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
   wherein the connecting portion is configured to connect the first portion to the second portion, wherein:
      the first, second, third and fourth planes are parallel to each other,
      the third cross-sectional area is smaller than the first, second and fourth cross-sectional areas, such that the first portion, second portion and connecting portion are prevented from travelling through the hole in the tissue portion in a direction perpendicular to the first, second and third planes, and
      the first portion is detachably connected to at least one of the connecting portion and the second portion.
31. The implantable medical device according to any one of the preceding claims, wherein the implantable medical device comprises a system for treating a patient having a disorder related to the patient's intestine, the system comprises
   an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
   the accumulator is adapted to be charged wirelessly with energy and to be arranged so as to supply energy directly or indirectly to at least one energy consuming part of said artificial intestine section.
32. The implantable medical device according to any one of the preceding claims, wherein the implantable medical device comprises an artificial flow control device implantable in the patient's body and adapted to control flow of the intestinal contents from said reservoir
   the artificial flow control device comprises at least one pump adapted to act on said intestinal wall so as to reduce the reservoir's volume in order to empty the reservoir.
33. The implantable medical device according to any one of the preceding claims, wherein the implantable medical device comprises an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
34. The implantable medical device according to any one of the preceding claims, wherein the implantable medical device comprises a drug delivery device for injecting a drug into a patient's body such as stimulation of penis erection wherein
   the drug is injected into the patient's body into at least one of both a right and left corpus cavernosum,
   two deep arteries of the right and left corpus cavernosum,
   muscle tissue regulating blood flow through the right and left corpus cavernosum, and
   tissue in close proximity to the left and right corpus cavernosum,
   wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
35. The implantable medical device according to any one of the preceding claims, wherein the implantable medical device comprises an implantable lubrication device comprising:
   a reservoir that stores a lubricating fluid and,
   a fluid connection that introduces the stored lubricating fluid into a damaged joint when the lubrication device is implanted in a patient's body.
   wherein the lubricating device is configured to be completely implanted into the patient's body such that a damaged joint can post-operatively be lubricated from within the patient's body,
   and wherein the operative supply of lubricating fluid to the damaged joint is controlled continuously, intermittently, periodically or depending on a physical parameter of the patient, such as a fluid level within the joint.
36. The implantable medical device according to any one of the preceding claims, wherein the implantable medical device comprises a device for bone adjustment in a mammal, comprising
   two or more anchoring devices for attaching to a bone in said mammal,
   an adjustment device for exerting force on said anchoring devices to adjust the distance between or orientation of at least two of said anchoring devices,
   wherein
   said anchoring devices and said adjustment device are implanted intramedullary in said mammal and wherein said adjustment device is constructed to postoperatively adjust said distance, and
   the adjustment is a lengthening of a bone, a healing of a fracture, a changing of a bone angle, a reshaping of a bone, a compression of a bone, a torsion of a bone, or a combination thereof.
37. The implantable medical device according to any one of the preceding claims, wherein the implantable medical device comprises an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
38. The implantable medical device according to any one of the preceding claims, wherein the implantable medical device comprises a fluid movement device for pumping hydraulic treatment fluid or body fluid, the fluid movement device being powered by an energy source and powered by an electrical or a hydraulic motor
   at least one connecting tube connected to the fluid movement device so that the fluid movement device and the tube form a drainage or hydraulic arrangement
   wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
39. The implantable medical device according to any one of the preceding claims, wherein the implantable medical device comprises an implantable device for improving the pump function of the heart of a human patient by applying an external force on the heart muscle, said device comprising
   at least one heart contacting organ, periodically exerting force onto the heart following the heart contractions and adding force thereto,
   a drive unit to create kinetic movement to be used by the heart contacting organ,
   a fixation device adapted to be mounted in a stable position to human bone allowing said drive unit and kinetic movement to get necessary contra force,
   wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
40. The implantable medical device according to any one of the preceding claims, wherein the implantable medical device comprises a male sexual impotence treatment prosthesis apparatus, comprising
   an operable prosthesis implantable in the cavities of the corpora cavernosa of an impotent patient to provide erect penile condition, when the prosthesis is operated,
   wherein an energy transmission device for wireless transmission of energy from outside the patient's body to inside the patient's body for use in connection with the operation of the prosthesis, when the prosthesis is implanted.
41. The implantable medical device according to any one of the preceding claims, wherein the implantable medical device comprises a device for treatment or monitoring of an aneurysm, comprising
   an implantable member adapted to hold fluid, the member being adapted to be placed in connection with a blood vessel having the aneurysm and to exercise a pressure on the aneurysm, wherein the controller functions as a control unit adapted to control pressure adjustments of the implantable member.
42. The implantable medical device according to any one of the preceding claims, wherein the implantable medical device comprises an implantable blood clot removal device for removing blood clots from the vascular system of a patient comprising:
   a blood flow passageway to be connected to the patient's vascular system to allow circulation of the patient's blood through the blood flow passageway,
   a filter provided in the blood flow passageway for collecting blood clots occurring in the blood flowing through the blood flow passageway, and
   a cleaning device for moving blood clots collected by the filter out of the blood flow passageway.
43. The implantable medical device according to any one of the preceding claims, wherein the implantable medical device comprises an implantable apparatus for treating urinary retention of a mammal patient comprising:
   an implantable powered member adapted exert a force from the outside on a selected part of the urinary bladder in order to discharge urine from the urinary bladder, and
   a control device for controlling the operation of the powered member.
44. The implantable medical device according to any one of the preceding claims, wherein the implantable medical device comprises a breast implant system comprises a plurality of chambers including one or more first fluid chambers and one or more second fluid chambers, wherein:
   at least the first fluid chamber is to be implanted in the human body to form part of a breast implant,
   the second fluid chambers is implanted to form part of the breast implant or implanted inside the patient's body remote from the breast implant,
   the first fluid chamber is interconnected with the second fluid chamber, such that fluid can be exchanged between the first and second fluid chambers, so as to change their respective fluid content.
45. The implantable medical device according to any one of the preceding claims, wherein the implantable medical device comprises an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
46. The implantable medical device according to any one of the preceding claims, wherein the implantable medical device comprises a system for treating a female patient to avoid or promote pregnancy comprising a restriction device adapted to postoperatively restrict and release an oviduct of the patient.
47. The implantable medical device according to any one of the preceding claims, wherein the implantable medical device comprises an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
48. The medical system according to claim 47, wherein the implantable medical device comprises an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
49. The implantable medical device according to any one of the preceding claims, wherein the implantable medical device comprises an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
50. The implantable medical device according to any one of the preceding claims, wherein the implantable medical device comprises an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
51. The implantable medical device according to any one of the preceding claims, wherein the implantable medical device comprises an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
52. The implantable medical device according to any one of the preceding claims, wherein the implantable medical device comprises a device for treating obesity of a patient, the device comprising:
   at least one operable stretching device implantable in the patient and adapted to stretch a portion of the patient's stomach wall, and
   an implantable control unit for automatically controlling the operable stretching device, when the control unit and stretching device are implanted, to stretch the stomach wall portion in connection with the patient eating such that satiety is created,
   wherein the stretching device comprises a first and a second engaging part, wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
53. The implantable medical device according to any one of the preceding claims, wherein the implantable medical device comprises an obesity treatment device comprising:
   at least one operable stretching device implantable in a patient and adapted to stretch a portion of the patient's stomach wall and
   an implantable operation device for operating the stretching device, when implanted, to stretch the stomach wall portion such that satiety is created,
   wherein the operable stretching device is adapted to be placed against the outside of the stomach wall.
54. The implantable medical device according to any one of the preceding claims, wherein the implantable medical device comprises a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
55. The implantable medical device according to any one of the preceding claims, wherein the implantable medical device comprises a device for treating obesity of a patient, the device comprising:
   a. at least one operable stretching device implantable in the patient operating via mechanical, pneumatic, and/or hydraulic action to stretch a portion of the patient's stomach wall, and
   b. an implantable control unit for automatically controlling the operable stretching device, when the control unit and stretching device are implanted, to stretch the stomach wall portion in connection with the patient eating such that satiety is created.

### Aspect 447AB Data_Packet_Encryption—Implant

1. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: a system for treating a female patient to avoid or promote pregnancy comprising a restriction device adapted to postoperatively restrict and release an oviduct of the patient.
18. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises: an obesity treatment device comprising:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. An implantable medical device configured to receive remote instructions from an external system, the implantable medical device comprising:
   a wireless receiver configured to receive wirelessly transmitted data packets from the external system,
   a computing unit configured to:
      verify the electronic signature, and
use a checksum provided in the data packet to verify the integrity of the instructions,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 447C Data Packet_Encryption-Method

1. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions.
2. The method according to claim 1, further comprising the steps of encrypting the data packet at the external system using a private key of the external system, and decrypting, at the implantable medical device, the data packet using a private key of the implantable medical device.
3. The method according to any one of claims 1 and 2, wherein the step of verifying the electronic signature comprises comparing the electronic signature with electronic signatures stored in the implantable medical device.
4. The method according to any one of claims 1-3, wherein the step of wirelessly sending the data packet to the implantable medical device comprises sending the data packet from a first external device to a second external device using wired communication and wirelessly sending the data packet from the second external device to the implantable medical device.
5. The method according to any one of claims 1-4, wherein the step of wirelessly sending the data packet to the implantable medical device comprises sending the data packet from a first external device to a second external device and further wirelessly sending the data packet from the second external device to the implantable medical device, and wherein the second external device transmits the data packet without changing the data packet.
6. The method according to any one of claims 1-5, wherein the step of wirelessly sending the data packet to the implantable medical device comprises sending the data packet from a first external device to a second external device and further wirelessly sending the data packet from the second external device to the implantable medical device, and wherein the second external device transmits the data packet without full decryption.
7. The method according to any one of the preceding claims, wherein the implantable medical device comprises a control program configured to control at least one function of the implantable medical device, and wherein the method further comprises altering the control program on the basis of the received instructions.
8. The method according to any one of the preceding claims, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
9. The method according to claim 8, wherein the implantable medical device comprises a set of stored parameter values, and wherein the method further comprises the step of verifying that the instructions for altering the at least one parameter will result in the at least one parameter being updated to a parameter value comprised in the set of stored parameter values.
10. The method according to any one of the preceding claims, wherein the step of wirelessly sending the data packet to the implantable medical device comprises: wirelessly sending the data packet from a first external device to a second external device using a first network protocol, and wirelessly sending the data packet from the second external device to the implantable medical device using a second network protocol.
11. The method according to any one of the preceding claims, wherein the step of wirelessly sending the data packet to the implantable medical device comprises: wirelessly sending the data packet from a first external device to a second external device using a first frequency band, and wirelessly sending the data packet from the second external device to the implantable medical device using a second frequency band.
12. The method according to any one of the preceding claims, wherein the step of wirelessly sending the data packet to the implantable medical device comprises:
   wirelessly sending the data packet from a first external device to a second external device using a first wireless technology, and
   wirelessly sending the data packet from the second external device to the implantable medical device using a second wireless technology, wherein the first wireless technology has an effective range being one of 2 times, 4 times, 8 times 20 times, 50 times or 100 times longer than the first wireless technology.
13. The method according to any one of the preceding claims, wherein the implantable medical device comprises a central unit, comprising a wireless transceiver, and a security module connected to the central unit, wherein the step of decrypting, at the implantable medical device, the data packet, comprises transferring the data packet from the central unit to the security module, and performing at least a portion of the decryption in the security module.
14. The method according to claim 13, wherein the security module comprises a set of rules for accepting communication from the central unit, and wherein the step of transferring the data packet from the receiving unit of the implant to the security module comprises verifying compliance with the set of rules.
15. The method according to claim 14, wherein wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
16. The method according to any one of the preceding claims, wherein the step of electronically signing the instructions at the external system comprises electronically signing the instructions at the external system using a private key of the external system.
17. The method according to claim 16, wherein the private key is a non-extractable key.
18. The method according to any one of claims 16 and 17, wherein the step of verifying the electronic signature comprises performing a proof of possession operation comprising the steps of:
   transmitting, form the medical device to the external system, a query based on a public key associated with the private of the external system,
   receiving, at the medical device, a response based on the possession of the private key in the external system, and
   verifying that the response based on the possession of the private key matches the query based on a public key.
19. The method according to any one of the preceding claims, wherein the step of forming the data packet is performed at a first external device, and wherein the step of electronically signing the instructions comprises electronically signing the instruction using a first private key, and wherein the method further comprises:
   transmitting the data packet from the first external device to a second external device,
   verifying, at the second external device, that the transmitter is a trusted transmitter,
   in response to the verification, electronically signing the data packet using a second private key, and
   transmitting the data packet from the second external device to the implantable medical device,
   and verifying, at the implantable medical device, the electronic signatures generated using the first and second private keys.
   using the checksum to verify the integrity of the instructions.
20. The method according to any one of the preceding claims, wherein the step of electronically signing the instructions and the checksum, at the external system, comprising signing the instructions and the checksum with the use of a key placed on a key device separate from at least one of the first and second external device.
21. The method according to any one of the preceding claims, wherein the step of electronically signing the instructions and the checksum, at the external system, comprising signing the instructions and the checksum with the use of a key placed on a key device comprising a wireless transmitter for wirelessly transmitting the at least one private key to at least one of the first and second external device.
22. The method according to any one of claims 20 and 21, wherein the step of electronically signing the instructions and the checksum, at the external system, further comprises signing the instructions and the checksum with the use of a second key placed on the key device or on a second key device, separate from at least one of the first and second external device.
23. The method according to any one of claims 21 and 22, wherein at least one of the key device and the second key device comprises at least one of: a key card, a wearable device and a handset.
24. The method according to any one of the preceding claims, further comprising the step of unlocking at least one of the first and second external device using user credentials provided to the first and/or second external device.
25. The method according to claim 24, wherein the step of unlocking at least one of the first and second external devices comprises unlocking at least one of the first and second external devices using a username and a password.
26. The method according to claim 24, wherein the step of unlocking at least one of the first and second external devices comprises unlocking at least one of the first and second external devices using a PIN-code.
27. The method according to claim 24, wherein the step of unlocking at least one of the first and second external devices comprises verifying, at the at least one first or second external devices, the user credentials by comparing the user credentials with user credentials stored in at least one of the first and second external devices.
28. The method according to claim 24, wherein the step of unlocking at least one of the first and second external devices comprises verifying, at the at least one first or second external devices, the user credentials by comparing the user credentials with user credentials stored in at least one of the first and second external devices by the manufacturer of at least one of the first and second external devices.
29. The method according to any one of claims 24-28, wherein the step of unlocking at least one of the first and second external devices comprises verifying, at the at least one first or second external devices, the user credentials by comparing the user credentials with user credentials stored as hardware or software in at least one of the first and second external devices.
30. The method according to any one of claims 24-29, wherein the step of unlocking at least one of the first and second external devices comprises verifying, at the at least one first or second external devices, the user credentials by communicating with a remote server.
31. The method according to any one of the preceding claims, wherein the method is performed without connection to the Internet.
32. The method according to any one of the preceding claims, wherein the method is configured to be performed independently of time.
33. The method according to any one of the preceding claims, wherein the first and second keys are different.
34. The method according to any one of the preceding claims, wherein at least one of the first and second keys are private.
35. The method according to claim 34, wherein the first and second private keys comprises at least one common element.
36. The method according to any one of claims 4-35, comprising unlocking at least one of the first and second external devices using at least one of the first and second private key.
37. The method according to any one of the preceding claims, wherein the step of electronically signing the instructions and the checksum is performed at a central server of the external system.
38. The method according to claim 37, further comprising:
   the central server being accessed by at least one healthcare professional, and
   the healthcare professional providing input to the central server for forming the instructions to be sent to the implantable medical device.
39. The method according to claim 37, further comprising the central server being accessed by at least one patient, such that the patient can provide input to the central server for verifying at least one of: the authenticity of the healthcare professional and the correctness of the instructions.
40. The method according to claim 38, further comprising the healthcare electronically signing the instructions at the central server.
41. The method according to claim 38, further comprising the patient electronically signing the instructions at the central server.
42. The method according to any one of claims 37-41, further comprising the steps of:
   verifying the authenticity of the first and second key at the central server, and
   electronically sign the instructions using the first and second key.
42. The method according to any one of the preceding claims, wherein the second key is a user key, and wherein the method comprises the steps of using the second key for at least one of:
   approving that communication is transmitted to the implantable medical device, and
   approving that a healthcare provider prepares an instruction to the implantable medical device.
43. The method according to claim 42, wherein the approval step can be performed by first or second external device.
44. The method according to any one of claims 4-43, wherein the first key is required to create an instruction to the implantable medical device and the second key is required to transmit the created instruction to the implantable medical device.
45. The method according to any one of claims 4-44, wherein at least one of the first and second external device comprises an input button, and wherein the method further comprises the step of pressing the button for verifying user presence.
46. The method according to claim 45, wherein the input button is placed on the second external device.
47. The method according to any one of the preceding claims, wherein the trental of the data packet comprises transmittal of:
   at least one instruction signed by a first key, and
   a public key including information about which root have created the public key.
48. The method according to any one of claims 4-47, further comprising enabling communication between the implantable medical device and at least one of the first and second medical device based on at least one password being provided to at least one of the first and second external device.
49. The method according to any one of claims 4-47, further comprising enabling communication between the implantable medical device and at least one of the first and second medical device based on two passwords being provided to at least one of the first and second external device.
50. The method according to claim 49, wherein the first password is a patient password and the second password is a healthcare provider passwords.
51. The method according to any one of the preceding claims, further comprising at least one of the first and second external devices performing a verification query operation with at least one of the first and second key devices, the verification query operation comprising:
   transmitting, from the first or second external devices, a query comprising a computational challenge to at least one of the first and second key device,
   receiving, at the first or second external devices, a response based on the transmitted computational challenge, and
   verifying, at the first or second external devices, the received response.
52. The method according to claim 51, wherein the verification query operation is in the form of a proof of possession operation comprising:
   receiving a public key of at least one of the first and second key devices, the public key being associated with a private key of the first or second key device,
   transmitting, from at least one of the first and second external devices, a computational challenge to the first or second key device, based on the public key received from the first or second key device,
   receiving a response from the first or second key device based on the possession of the private key in the first or second key device, and
   verifying that the response based on the possession of the private key matches the query based on a public key.

### Aspect 447CB Data_Packet_Encryption—Method

1. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion,
wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
2. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: a system for treating a female patient to avoid or promote pregnancy comprising a restriction device adapted to postoperatively restrict and release an oviduct of the patient.
18. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 447D Single-Use_Codes_Encryption

1. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes.
2. The method according to claim 1, further comprising the steps of:
   wirelessly sending position information from the external device to the implantable medical device, and
   using the information at the implantable medical device for selecting the code from the list of codes.
3. The method according to claim 1, wherein the step of encrypting, at the external system, the instructions using a code from a position on the list of codes comprises selecting the code on a current position on the list of codes, wherein the method further comprises the step of updating the current position to a new current position after using the code.
4. The method according to any one of claims 1 and 3, wherein the step of decrypting, at the implantable medical device, the instructions using a code from a position on the list of codes comprises selecting the code on a current position on the list of codes, wherein the method further comprises the step of updating the current position to a new current position after using the code.
5. The method according to any one of claims 3 and 4, wherein the current position comprises a number and wherein the step of updating the current position comprises updating the number to a sequential number.
6. The method according to any one of claims 1-5, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without changing the encrypted instructions.
7. The method according to any one of claims 1-6, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without full decryption.
8. The method according to any one of the preceding claims, wherein the implantable medical device comprises a control program configured to control at least one function of the implantable medical device, and wherein the method further comprises altering the control program on the basis of the received instructions.
9. The method according to any one of the preceding claims, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
10. The method according to claim 9, wherein the implantable medical device comprises a set of stored parameter values, and wherein the method further comprises the step of verifying that the instructions for altering the at least one parameter will result in the at least one parameter being updated to a parameter value comprised in the set of stored parameter values.
11. The method according to any one of the preceding claims, wherein the step of wirelessly sending the encrypted instructions to the implantable medical device comprises:
   wirelessly sending the encrypted instructions from a first external device to a second external device using a first network protocol, and
   wirelessly sending the encrypted instructions from the second external device to the implantable medical device using a second network protocol.
11. The method according to any one of the preceding claims, wherein the step of wirelessly sending the encrypted instructions to the implantable medical device comprises:
   wirelessly sending the encrypted instructions from a first external device to a second external device using a first frequency band, and
   wirelessly sending the encrypted instructions from the second external device to the implantable medical device using a second frequency band.
12. The method according to any one of the preceding claims, wherein the step of wirelessly sending the encrypted instructions to the implantable medical device comprises:
   wirelessly sending the encrypted instructions from a first external device to a second external device using a first wireless technology, and
   wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.
13. The method according to any one of the preceding claims, wherein the implantable medical device comprises a central unit, comprising a wireless transceiver, and a security module connected to the central unit, wherein the step of decrypting, at the implantable medical device, the encrypted instructions, comprises transferring the encrypted instructions from the central unit to the security module, and performing at least a portion of the decryption in the security module.
14. The method according to claim 13, wherein the security module comprises a set of rules for accepting communication from the central unit, and wherein the step of transferring the encrypted instructions from the receiving unit of the implant to the security module comprises verifying compliance with the set of rules.
15. The method according to claim 14, wherein wireless transceiver is configured to be placed in an off-mode, in which no wireless communication can be received by the wireless transceiver, and wherein the set of rules comprises a rule stipulating that communication from the central unit is only accepted at the security module when the wireless transceiver is placed in the off-mode.
16. The method according to any one of the preceding claims, wherein the step of electronically signing the instructions at the external system comprises electronically signing the instructions at the external system using a private key of the external system.
17. The method according to claim 16, wherein the private key is a non-extractable key.
18. The method according to any one of the preceding claims, wherein the step of wirelessly sending the encrypted instructions to the implantable medical device comprises:
   wirelessly sending the encrypted instructions from a first external device to a second external device using a first wireless technology, and
   wirelessly sending the encrypted instructions from the second external device to the implantable medical device using a second wireless technology, wherein the first wireless technology has an effective range being one of: 2 times, 4 times, 8 times 20 times, 50 times or 100 times longer than the first wireless technology.

### Aspect 447DB Single Use Codes

1. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
2. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: a system for treating a female patient to avoid or promote pregnancy comprising a restriction device adapted to postoperatively restrict and release an oviduct of the patient.
18. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.

### Aspect 377-Electro_Subcutaneous_Control_Pop-Rivet2_Bellows

1. An implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion, and
a hermetic seal arrangement configured to enclose the connecting portion so as to prevent fluid from the patient to enter the connecting portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes, and
the connecting portion comprises a flexible structure enabling the connecting portion to flex.
2. The implantable energized medical device according to aspect 1, wherein the flexible structure is configured to allow the connecting portion to flex in more than one direction.
3. The implantable energized medical device according to aspect 2, wherein the flexible structure is configured to allow the connecting portion to flex in all directions.
4. The implantable energized medical device according to any one of the preceding aspects, wherein the flexible structure comprises a bellows.
5. The implantable energized medical device according to aspect 4, wherein the bellows is a metallic bellows.
6. The implantable energized medical device according to aspect 5, wherein the metallic bellows is welded.
7. The implantable energized medical device according to any one of aspects 4 to 6, wherein the bellows is a titanium bellows.
8. The implantable energized medical device according to any one of aspects 4 to 8, wherein the bellows form part of the hermetic seal arrangement.
9. The implantable energized medical device according to any one of the preceding aspects, wherein the flexible structure comprises elevated and lowered portions enabling said flexing of the connecting portion.
10. The implantable energized medical device according to aspect 9, wherein the elevated and lowered portions are configured to enable the connecting portion to be compressed and/or expanded.
11. The implantable energized medical device according to any one of the preceding aspects, wherein the flexible structure has a substantially cylindrical shape.
12. The implantable energized medical device according to any one of the preceding aspects, wherein the flexible structure is configured to seal against the first portion and/or the second portion.
13. The implantable energized medical device according to any one of the preceding aspects, wherein the connecting portion and the second portion are hermetically sealed from the first portion.
14. The implantable energized medical device according to aspect 13, wherein the hermetic seal arrangement encloses the connecting portion and the second portion so as to hermetically seal the connecting portion and the second portion from the first portion.
15. The implantable energized medical device according to any one of the preceding aspects, wherein the first portion comprises a first wireless energy receiver for receiving energy transmitted wirelessly by an external wireless energy transmitter, and an internal wireless energy transmitter configured to transmit energy wirelessly to the second portion, and the second portion comprises a second wireless energy receiver configured to receive energy transmitted wirelessly by the internal wireless energy transmitter.
16. The implantable energized medical device according to aspect 15, wherein the first portion comprises a first energy storage unit connected to the first wireless energy receiver.
17. The implantable energized medical device according to any one of aspects 15 and 16, wherein the second portion comprises a second energy storage unit connected to the second wireless energy receiver.
18. The implantable energized medical device according to aspect 17, wherein at least one of the first and second energy storage unit is a solid-state battery.
19. The implantable energized medical device according to aspect 18, wherein the solid-state battery is a thionyl-chloride battery.
20. The implantable energized medical device according to any one of aspects 17-19, wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
21. The implantable energized medical device according to any one of aspects 15-20, wherein the first portion comprises a first controller comprising at least one processing unit.
22. The implantable energized medical device according to any one of aspects 15-21, wherein the second portion comprises a second controller comprising at least one processing unit.
23. The implantable energized medical device according to any one of aspects 21 and 22, wherein at least one of the first and second controller is connected to a wireless transceiver for communicating wirelessly with an external device.
24. The implantable energized medical device according to any one of aspects 21 and 22, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
25. The implantable energized medical device according to aspect 24, wherein the second controller is connected to the second wireless communication receiver for receiving wireless communication from the first portion.
26. The implantable energized medical device according to any one of aspects 15-25, wherein the first wireless energy receiver comprises a first coil and the wireless energy transmitter comprises a second coil.
27. The implantable energized medical device according to any one of aspects 15-26, wherein the first portion comprises a combined coil, wherein the combined coil is configured to receive energy wirelessly from an external wireless energy transmitter, and transmit energy wirelessly to the second wireless receiver of the second portion.
28. The implantable energized medical device according to any one of aspects 26 and 27, wherein at least one of the coils are embedded in a ceramic material.
29. The implantable energized medical device according to any one of the preceding aspects, further comprising a housing configured to enclose at least the first portion, and wherein a first portion of the housing is made from titanium and a second portion of the housing is made from a ceramic material.
30. The implantable energized medical device according to aspect 29, wherein the portion of the housing made from a ceramic material comprises at least one coil embedded in the ceramic material.
31. The implantable energized medical device according to any one of the preceding aspects, further comprising a housing configured to enclose at least the second portion, and wherein a first portion of the housing is made from titanium and a second portion of the housing is made from a ceramic material.
32. The implantable energized medical device according to aspect 31, wherein the portion of the housing made from a ceramic material comprises at least one coil embedded in the ceramic material.
33. The implantable energized medical device according to any one of the preceding aspects, wherein the first portion is detachably connected to at least one of the second portion and the connecting portion.
34. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
35. The implantable energized medical device according to any one of the preceding aspects, wherein a connecting interface between the connecting portion and the second portion is excentric with respect to the second portion.
36. The implantable energized medical device according to any one of the preceding aspects, wherein a connecting interface between the connecting portion and the first portion is excentric with respect to the first portion.
37. The implantable energized medical device according to any one of the preceding aspects, wherein the second portion has a first end and a second end opposing the first end, wherein the second portion has a length between the first and second end.
38. The implantable energized medical device according to aspect 37, wherein the first end and second end are separated in a direction parallel to the second plane.
39. The implantable energized medical device according to aspect 37 or 38, wherein the first and second ends comprise an elliptical point respectively.
40. The implantable energized medical device according to any one of aspects 37-39, wherein the first and second ends comprise a hemispherical end cap respectively.
41. The implantable energized medical device according to any one of aspects 37-40, wherein the second portion has at least one circular cross-section along the length between the first and second end.
42. The implantable energized medical device according to any one of aspects 37-41, wherein the second portion has at least one oval cross-section along the length between the first and second end.
43. The implantable energized medical device according to any one of aspects 37-42, wherein the second portion has at least one elliptical cross-section along the length between the first and second end.
44. The implantable energized medical device according to any one of the preceding aspects, further comprising a gear arrangement and an electric motor, wherein the gear arrangement is configured to reduce the velocity and increase the force of the movement generated by the electric motor.
45. The implantable energized medical device according to aspect 44, wherein the gear arrangement is configured to transfer a force with a high velocity into a stronger force with lower velocity.
46. The implantable energized medical device according to aspect 44 or 45, wherein the gear arrangement is configured to transfer a rotating force into a linear force.
47. The implantable energized medical device according to any one of aspects 44-46, wherein the gear arrangement comprises a gear system.
48. The implantable energized medical device according to any one of aspects 44-47, wherein the second portion comprises a magnetic coupling for transferring mechanical work from the electric motor through one of:
a barrier separating a first chamber of the second portion from a second chamber of the second portion,
a housing enclosing at least the second portion.
49. The implantable energized medical device according to any one of the preceding aspects, wherein the second portion comprises at least one hydraulic pump.
50. The implantable energized medical device according to aspect 49, wherein the hydraulic pump comprises a pump comprising at least one compressible hydraulic reservoir.

### Aspect 434_Electro_Subcutaneous_Control_Pop-Rivet2_Decreasing-Area

1. An implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and being configured to connect the first portion to the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes,
wherein the connecting portion and the second portion are configured to form a unit having a central axis extending from a first end of said unit to a second end of said unit, the first end being proximal to the first portion and the second end being distal to the first portion,
wherein a physical footprint of said unit perpendicular to the central axis decreases continuously or stepwise from the first end to the second end of said unit.
2. The implantable energized medical device according to aspect 1, wherein said physical footprint comprises a cross-sectional area perpendicular to the central axis.
3. The implantable energized medical device according to aspect 1 or 2, wherein the connecting portion and the second portion are one of:
configured to reversibly connect to each other to form said unit; or
configured to irreversibly connect to each other to form said unit; or
configured as a single body forming said unit.
4. The implantable energized medical device according to any one of the preceding aspects, wherein said unit comprises an angled section forming a bend in said unit.
5. The implantable energized medical device according to aspect 4, wherein the bend is between 15° and 165°, such as between 30° and 150°, such as between 45° and 135°, such as substantially 90°.
6. The implantable energized medical device according to any one of the preceding aspects, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
7. The implantable energized medical device according to aspect 6, wherein the first portion comprises a first energy storage unit connected to the first wireless energy receiver.
8. The implantable energized medical device according to any one of aspects 6 and 7, wherein the second portion comprises a second energy storage unit connected to the second wireless energy receiver.
9. The implantable energized medical device according to aspect 8, wherein at least one of the first and second energy storage unit is a solid-state battery.
10. The implantable energized medical device according to aspect 9, wherein the solid-state battery is a thionyl-chloride battery.
11. The implantable energized medical device according to any one of aspects 8-10, wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
12. The implantable energized medical device according to any one of aspects 6-11, wherein the first portion comprises a first controller comprising at least one processing unit.
13. The implantable energized medical device according to any one of aspects 6-12, wherein the second portion comprises a second controller comprising at least one processing unit.
14. The implantable energized medical device according to any one of aspects 12 and 13, wherein at least one of the first and second controller is connected to a wireless transceiver for communicating wirelessly with an external device.
15. The implantable energized medical device according to any one of aspects 12 and 13, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
16. The implantable energized medical device according to aspect 15, wherein the second controller is connected to the second wireless communication receiver for receiving wireless communication from the first portion.
17. The implantable energized medical device according to any one of aspects 6-16, wherein the first wireless energy receiver comprises a first coil and the wireless energy transmitter comprises a second coil.
18. The implantable energized medical device according to any one of aspects 6-17, wherein the first portion comprises a combined coil, wherein the combined coil is configured to receive energy wirelessly from an external wireless energy transmitter, and transmit energy wirelessly to the second wireless receiver of the second portion.
19. The implantable energized medical device according to any one of aspects 17 and 18, wherein at least one of the coils are embedded in a ceramic material.
20. The implantable energized medical device according to any one of the preceding aspects, further comprising a housing configured to enclose at least the first portion, and wherein a first portion of the housing is made from titanium and a second portion of the housing is made from a ceramic material.
21. The implantable energized medical device according to aspect 20, wherein the portion of the housing made from a ceramic material comprises at least one coil embedded in the ceramic material.
22. The implantable energized medical device according to any one of the preceding aspects, further comprising a housing configured to enclose at least the second portion, and wherein a first portion of the housing is made from titanium and a second portion of the housing is made from a ceramic material.
23. The implantable energized medical device according to aspect 22, wherein the portion of the housing made from a ceramic material comprises at least one coil embedded in the ceramic material.
24. The implantable energized medical device according to any one of the preceding aspects, wherein the first portion is detachably connected to at least one of the second portion and the connecting portion.
25. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
26. The implantable energized medical device according to any one of the preceding aspects, wherein a connecting interface between the connecting portion and the second portion is excentric with respect to the second portion.
27. The implantable energized medical device according to any one of the preceding aspects, wherein a connecting interface between the connecting portion and the first portion is excentric with respect to the first portion.
28. The implantable energized medical device according to any one of the preceding aspects, wherein the second portion has a first end and a second end opposing the first end, wherein the second portion has a length between the first and second end.
29. The implantable energized medical device according to aspect 28, wherein the first end and second end are separated in a direction parallel to the second plane.
30. The implantable energized medical device according to aspect 28 or 29, wherein the first and second ends comprise an elliptical point respectively.
31. The implantable energized medical device according to any one of aspects 28-30, wherein the first and second ends comprise a hemispherical end cap respectively.
32. The implantable energized medical device according to any one of aspects 28-31, wherein the second portion has at least one circular cross-section along the length between the first and second end.
33. The implantable energized medical device according to any one of aspects 28-32, wherein the second portion has at least one oval cross-section along the length between the first and second end.
34. The implantable energized medical device according to any one of aspects 28-33, wherein the second portion has at least one elliptical cross-section along the length between the first and second end.
35. The implantable energized medical device according to any one of the preceding aspects, further comprising a gear arrangement and an electric motor, wherein the gear arrangement is configured to reduce the velocity and increase the force of the movement generated by the electric motor.
36. The implantable energized medical device according to aspect 35, wherein the gear arrangement is configured to transfer a force with a high velocity into a stronger force with lower velocity.
37. The implantable energized medical device according to aspect 35 or 36, wherein the gear arrangement is configured to transfer a rotating force into a linear force.
38. The implantable energized medical device according to any one of aspects 35-37, wherein the gear arrangement comprises a gear system.
39. The implantable energized medical device according to any one of aspects 35-38, wherein the second portion comprises a magnetic coupling for transferring mechanical work from the electric motor through one of:
a barrier separating a first chamber of the second portion from a second chamber of the second portion,
a housing enclosing at least the second portion.
40. The implantable energized medical device according to any one of the preceding aspects, wherein the second portion comprises at least one hydraulic pump.
41. The implantable energized medical device according to aspect 40, wherein the hydraulic pump comprises a pump comprising at least one compressible hydraulic reservoir.

Alternatively, aspect 1 of this aspect group (ASPECT_434) may read as follows, and the dependent aspects 2-41 may apply also to this alternative:

An implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and being configured to connect the first portion to the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes,
wherein the connecting portion is configured to extend between the first portion and the second portion along a central extension axis, and wherein the second portion is configured to extend in a length direction being divergent with the central extension axis, and wherein the connecting portion has a decreasing cross-sectional area in a direction from the first portion towards the second portion along the central extension axis, wherein a largest cross-sectional area of the second portion in the length direction is smaller than a smallest cross-sectional area of the connecting portion in said direction from the first portion towards the second portion along the central extension axis, and wherein the second portion further has a decreasing cross-sectional area in the length direction from a first end of the second portion proximal to the connecting portion to a second end of the second portion distal to the connecting portion.

### Aspect 435_Electro_Subcutaneous_Control_Pop-Rivet2_Electric-Motor-Orientation

1. An implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion, and
an electric motor,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes,
at least part of the electric motor is arranged within the connecting portion.
2. The implantable energized medical device according to aspect 1, wherein the electric motor is arranged within the connecting portion within an imaginary boundary defined by the first surface of the first portion extending through the connecting portion.
3. The implantable energized medical device according to aspect 1 or 2, wherein the electric motor is arranged within the connecting portion within an imaginary boundary defined by the second surface of the second portion extending through the connecting portion.
4. The implantable energized medical device according to aspect 1, wherein the electric motor is fully arranged in the connecting portion within imaginary boundaries defined by the first surface of the first portion extending through the connecting portion and the second surface of the second portion extending through the connecting portion respectively.
3. The implantable energized medical device according to aspect 1 or 2, wherein the electric motor is arranged such that its longest dimension extends in a direction substantially perpendicular to the first, second and third cross-sectional areas.
4. The implantable energized medical device according to any one of the preceding aspects, wherein the electric motor is arranged such that its longest dimension extends in a direction between the first portion and the second portion.
5. The implantable energized medical device according to aspect 5, wherein the worm drive is configured to transfer mechanical force from the electric motor to an implantable body engaging portion being external to the implantable energized medical device.
6. The implantable energized medical device according to any one of the preceding aspects, wherein the electric motor extends through the connecting portion into the first portion and/or the second portion.
7. The implantable energized medical device according to aspect 6, wherein the electric motor extends through an imaginary boundary defined by the first surface of the first portion extending through the connecting portion.
8. The implantable energized medical device according to aspect 6, wherein the electric motor extends through an imaginary boundary defined by the second surface of the second portion extending through the connecting portion.
9. The implantable energized medical device according to aspect 6, wherein the electric motor extends through imaginary boundaries defined by the first surface of the first portion extending through the connecting portion and the second surface of the second portion extending through the connecting portion respectively.
10. The implantable energized medical device according to any one of the preceding aspects, further comprising a gear arrangement operatively connected to the electric motor wherein the gear arrangement is partly or fully arranged in one of the first portion and the second portion.
11. The implantable energized medical device according to aspect 10, wherein the gear arrangement is arranged within the connecting portion within an imaginary boundary defined by the first surface of the first portion extending through the connecting portion.
12. The implantable energized medical device according to aspect 10 or 11, wherein the gear arrangement is arranged within the connecting portion within an imaginary boundary defined by the second surface of the second portion extending through the connecting portion.
13. The implantable energized medical device according to any one of aspects 10 to 12, wherein the gear arrangement is fully arranged in the connecting portion within imaginary boundaries defined by the first surface of the first portion extending through the connecting portion and the second surface of the second portion extending through the connecting portion respectively.
14. The implantable energized medical device according to any one of aspects 10 to 13, wherein the gear arrangement extends through the connecting portion into the first portion and/or the second portion.
15. The implantable energized medical device according to aspect 14, wherein the gear arrangement extends through an imaginary boundary defined by the first surface of the first portion extending through the connecting portion.
16. The implantable energized medical device according to aspect 14, wherein the gear arrangement extends through an imaginary boundary defined by the second surface of the second portion extending through the connecting portion.
17. The implantable energized medical device according to aspect 14, wherein the gear arrangement extends through imaginary boundaries defined by the first surface of the first portion extending through the connecting portion and the second surface of the second portion extending through the connecting portion respectively.
18. The implantable energized medical device according to any one of aspects 10 to 17, wherein the gear arrangement is configured to transfer mechanical force from the electric motor to an implantable body engaging portion being external to the implantable energized medical device.
19. The implantable energized medical device according to any one of aspects 10 to 18, wherein the gear arrangement is a worm drive or comprises a worm drive.
20. The implantable energized medical device according to any one of the preceding aspects, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
21. The implantable energized medical device according to aspect 20, wherein the first portion comprises a first energy storage unit connected to the first wireless energy receiver.
22. The implantable energized medical device according to any one of aspects 20 and 21, wherein the second portion comprises a second energy storage unit connected to the second wireless energy receiver.
23. The implantable energized medical device according to aspect 22, wherein at least one of the first and second energy storage unit is a solid-state battery.
24. The implantable energized medical device according to aspect 23, wherein the solid-state battery is a thionyl-chloride battery.
25. The implantable energized medical device according to any one of aspects 22-24,
wherein: the first wireless energy receiver is configured to receive energy transmitted wirelessly by the external wireless energy transmitter and store the received energy in the first energy storage unit, the internal wireless energy transmitter is configured to wirelessly transmit energy stored in the first energy storage unit to the second wireless energy receiver, and the second wireless energy receiver is configured to receive energy transmitted wirelessly by the internal wireless energy transmitter and store the received energy in the second energy storage unit.
26. The implantable energized medical device according to any one of aspects 20-25, wherein the first portion comprises a first controller comprising at least one processing unit.
27. The implantable energized medical device according to any one of aspects 20-26, wherein the second portion comprises a second controller comprising at least one processing unit.
28. The implantable energized medical device according to any one of aspects 26 and 27, wherein at least one of the first and second controller is connected to a wireless transceiver for communicating wirelessly with an external device.
29. The implantable energized medical device according to any one of aspects 26 and 27, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
30. The implantable energized medical device according to aspect 29, wherein the second controller is connected to the second wireless communication receiver for receiving wireless communication from the first portion.
31. The implantable energized medical device according to any one of aspects 20-30, wherein the first wireless energy receiver comprises a first coil and the wireless energy transmitter comprises a second coil.
32. The implantable energized medical device according to any one of aspects 20-31, wherein the first portion comprises a combined coil, wherein the combined coil is configured to receive energy wirelessly from an external wireless energy transmitter, and transmit energy wirelessly to the second wireless receiver of the second portion.
33. The implantable energized medical device according to any one of aspects 31 and 32, wherein at least one of the coils are embedded in a ceramic material.
34. The implantable energized medical device according to any one of the preceding aspects, further comprising a housing configured to enclose at least the first portion, and wherein a first portion of the housing is made from titanium and a second portion of the housing is made from a ceramic material.
35. The implantable energized medical device according to aspect 34, wherein the portion of the housing made from a ceramic material comprises at least one coil embedded in the ceramic material.
36. The implantable energized medical device according to any one of the preceding aspects, further comprising a housing configured to enclose at least the second portion, and wherein a first portion of the housing is made from titanium and a second portion of the housing is made from a ceramic material.
37. The implantable energized medical device according to aspect 36, wherein the portion of the housing made from a ceramic material comprises at least one coil embedded in the ceramic material.
38. The implantable energized medical device according to any one of the preceding aspects, wherein the first portion is detachably connected to at least one of the second portion and the connecting portion.
39. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
40. The implantable energized medical device according to any one of the preceding aspects, wherein a connecting interface between the connecting portion and the second portion is excentric with respect to the second portion.
41. The implantable energized medical device according to any one of the preceding aspects, wherein a connecting interface between the connecting portion and the first portion is excentric with respect to the first portion.
42. The implantable energized medical device according to any one of the preceding aspects, wherein the second portion has a first end and a second end opposing the first end, wherein the second portion has a length between the first and second end.
43. The implantable energized medical device according to aspect 42, wherein the first end and second end are separated in a direction parallel to the second plane.
44. The implantable energized medical device according to aspect 42 or 43, wherein the first and second ends comprise an elliptical point respectively.
45. The implantable energized medical device according to any one of aspects 42-44, wherein the first and second ends comprise a hemispherical end cap respectively.
46. The implantable energized medical device according to any one of aspects 42-45, wherein the second portion has at least one circular cross-section along the length between the first and second end.
47. The implantable energized medical device according to any one of aspects 42-46, wherein the second portion has at least one oval cross-section along the length between the first and second end.
48. The implantable energized medical device according to any one of aspects 42-47, wherein the second portion has at least one elliptical cross-section along the length between the first and second end.
49. The implantable energized medical device according to any one of the preceding aspects, wherein the gear arrangement is configured to reduce the velocity and increase the force of the movement generated by the electric motor.
50. The implantable energized medical device according to aspect 49, wherein the gear arrangement is configured to transfer a force with a high velocity into a stronger force with lower velocity.
51. The implantable energized medical device according to aspect 49 or 50, wherein the gear arrangement is configured to transfer a rotating force into a linear force.
52. The implantable energized medical device according to any one of aspects 49-51, wherein the gear arrangement comprises a gear system.
53. The implantable energized medical device according to any one of aspects 49-52, wherein the second portion comprises a magnetic coupling for transferring mechanical work from the electric motor through one of:
a barrier separating a first chamber of the second portion from a second chamber of the second portion,
a housing enclosing at least the second portion.
54. The implantable energized medical device according to any one of the preceding aspects, wherein the second portion comprises at least one hydraulic pump.
55. The implantable energized medical device according to aspect 54, wherein the hydraulic pump comprises a pump comprising at least one compressible hydraulic reservoir.

### Aspect 436_Electro_Subcutaneous_Control_Pop-Rivet2_First-Portion

1. An implantable energized medical device configured to be held in position by a tissue portion of a patient, the medical device comprising:
a first portion configured to be placed on a first side of the tissue portion, the first portion comprising a first surface configured to face a first tissue surface of the first side of the tissue portion, the first portion being further configured to connect, directly or indirectly, to a second portion placed on a second side of the tissue portion opposing the first side,
wherein the first portion comprises an internal wireless energy transmitter configured to transmit energy wirelessly to the second portion.
2. The implantable energized medical device according to aspect 1, wherein the first portion is configured to connect, directly or indirectly, to the second portion, via a connecting portion configured to extend through a hole in the tissue portion, the hole extending between the first side of the tissue portion and the second side of the tissue portion.
3. The implantable energized medical device according to aspect 2, further comprising the connecting portion.
4. The implantable energized medical device according to aspect 3, wherein the connecting portion is integrally formed with the first portion.
5. The implantable energized medical device according to aspect 3, wherein the connecting portion is a separate component with regard to the first portion, the connecting portion being configured to be connected to the first portion.
6. The implantable energized medical device according to any one of aspects 2-5, wherein the first portion has a first cross-sectional area in a first plane and the connecting portion has a second cross-sectional area in a second plane, wherein the first and second planes are parallel to each other, wherein the second cross-sectional area is smaller than the first cross-sectional area, such that the first portion and the second portion are prevented from travelling through the hole in the tissue portion in a direction perpendicular to the first and second planes.
7. The implantable energized medical device according to any one of the preceding aspects, wherein the first portion is configured to detachably connect, directly or indirectly, to the second portion.
8. The implantable energized medical device according to any one of the preceding aspects, wherein the first portion comprises a first wireless energy receiver for receiving energy transmitted wirelessly by an external wireless energy transmitter.
9. The implantable energized medical device according to aspect 8, wherein the first portion comprises a first energy storage unit connected to the first wireless energy receiver.
10. The implantable energized medical device according to aspect 9, wherein the first energy storage unit is a solid-state battery.
11. The implantable energized medical device according to aspect 10, wherein the solid-state battery is a thionyl-chloride battery.
12. The implantable energized medical device according to any one of aspects 8-11, wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to a second wireless energy receiver in the second portion.
13. The implantable energized medical device according to any one of the preceding aspects, wherein the first portion comprises a first controller comprising at least one processing unit.
14. The implantable energized medical device according to aspect 13, wherein the first controller is connected to a wireless transceiver for communicating wirelessly with an external device.
15. The implantable energized medical device according to aspect 13, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
16. The implantable energized medical device according to any one of aspects 8-15, wherein the first wireless energy receiver comprises a first coil and the wireless energy transmitter comprises a second coil.
17. The implantable energized medical device according to any one of aspects 8-16, wherein the first portion comprises a combined coil, wherein the combined coil is configured to receive energy wirelessly from an external wireless energy transmitter, and transmit energy wirelessly to the second wireless receiver of the second portion.
18. The implantable energized medical device according to any one of aspects 16 and 17, wherein at least one of the coils are embedded in a ceramic material.
19. The implantable energized medical device according to aspect 3, wherein the connecting portion comprises a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
20. The implantable energized medical device according to any one of the preceding aspects, wherein a connecting interface between the connecting portion and the first portion is excentric with respect to the first portion.

### Aspect 437_Electro_Subcutaneous_Control_Pop-Rivet2_Remote-Parts

1. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit;
an implantable reservoir configured to hold a fluid;
an implantable pump configured to transfer fluid to and from the implantable reservoir and the body engaging implant respectively via a conduit; and
an implantable electric motor connected to the implantable energy storage unit, the implantable electric motor being configured to operate the implantable pump;
wherein the implantable energy storage unit, the implantable reservoir, the implantable pump and the implantable electric motor are arranged externally to the implantable energized medical device.
2. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
an implantable energy storage unit arranged in the first portion, the connecting portion or the second portion;
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable reservoir configured to hold a fluid;
an implantable pump configured to transfer fluid to and from the implantable reservoir and the body engaging implant respectively via a conduit; and
an implantable electric motor connected to the implantable energy storage unit, the implantable electric motor being configured to operate the implantable pump; wherein the implantable reservoir, the implantable pump and the implantable electric motor are arranged externally to the implantable energized medical device.
3. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
      a second portion configured to be placed on a second side of
the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion, and
an implantable electric motor arranged in the first portion, the connecting portion or the second portion;
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit;
an implantable reservoir configured to hold a fluid;
an implantable pump configured to transfer fluid to and from the implantable reservoir and the body engaging implant respectively via a conduit; wherein the implantable electric motor is connected to the implantable energy storage unit, and the implantable electric motor is configured to operate the implantable pump;
wherein the implantable energy storage unit, the implantable reservoir and the implantable pump are arranged externally to the implantable energized medical device.
4. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
   an implantable reservoir configured to hold a fluid, the implantable reservoir being arranged in the first portion, the connecting portion or the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit;
an implantable pump configured to transfer fluid to and from the reservoir and the body engaging implant respectively via a conduit; and
an implantable electric motor connected to the implantable energy storage unit, the implantable electric motor being configured to operate the implantable pump;
wherein the implantable energy storage unit, the implantable pump and the implantable electric motor are arranged externally to the implantable energized medical device.
5. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion, and
an implantable pump arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit;
an implantable reservoir configured to hold a fluid; and
an implantable electric motor connected to the implantable energy storage unit, the implantable electric motor being configured to operate the implantable pump; wherein the implantable pump is configured to transfer fluid to and from the implantable reservoir and the body engaging implant respectively via a conduit;
wherein the implantable energy storage unit, the implantable reservoir, and the implantable electric motor are arranged externally to the implantable energized medical device.
6. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion, and
a connecting portion configured to be placed through a hole in the tissue portion extending between the first and second sides of the tissue portion, the connecting portion having a third cross-sectional area in a third plane and being configured to connect the first portion to the second portion, wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit; and
an implantable electric motor connected to the implantable energy storage unit, the implantable electric motor being configured to operate the body engaging implant; wherein the implantable energy storage unit and the implantable electric motor are arranged externally to the implantable energized medical device.
7. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion,
an implantable energy storage unit arranged in the first portion, the connecting portion or the second portion, and
an implantable electric motor arranged in the first portion, the connecting portion or the second portion, the implantable electric motor being connected to the implantable energy storage unit,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable reservoir configured to hold a fluid; and
an implantable pump configured to transfer fluid to and from the implantable reservoir and the body engaging implant respectively via a conduit; wherein the implantable electric motor is configured to operate the implantable pump; wherein the implantable reservoir and the implantable pump are arranged externally to the implantable energized medical device.
8. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion,
an implantable reservoir configured to hold a fluid arranged in the first portion, the connecting portion or the second portion, and
an implantable electric motor arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ; an implantable energy storage unit, and
an implantable pump configured to transfer fluid to and from the implantable reservoir and the body engaging implant respectively via a conduit; wherein the implantable electric motor is connected to the implantable energy storage unit and configured to operate the implantable pump; wherein the implantable energy storage unit and the implantable pump are arranged externally to the implantable energized medical device.
9. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion,
an implantable reservoir configured to hold a fluid, the implantable reservoir being arranged in the first portion, the connecting portion or the second portion, and
an implantable pump arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit; and
an implantable electric motor connected to the implantable energy storage unit, the implantable electric motor being configured to operate the implantable pump; wherein the implantable pump is configured to transfer fluid to and from the implantable reservoir and the body engaging implant respectively via a conduit; and wherein the implantable energy storage unit and the implantable electric motor are arranged externally to the implantable energized medical device.
10. A system comprising
an implantable energized medical device configured to be held in position by a tissue portion of a patient, the medical device comprising:
a first portion configured to be placed on a first side of the tissue portion, the first portion having a first cross-sectional area in a first plane and comprising a first surface configured to face a first tissue surface of the first side of the tissue portion,
a second portion configured to be placed on a second side of the tissue portion, the second side opposing the first side, the second portion having a second cross-sectional area in a second plane and comprising a second surface configured to engage a second tissue surface of the second side of the tissue portion,
a connecting portion configured to be placed through a hole in the tissue portion extending between the first and second sides of the tissue portion, the connecting portion having a third cross-sectional area in a third plane and being configured to connect the first portion to the second portion,
an implantable pump arranged in the first portion, the connecting portion or the second portion, and
an implantable energy storage unit arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable reservoir configured to hold a fluid;
an implantable electric motor connected to the implantable energy storage unit, the implantable electric motor being configured to operate the implantable pump; wherein the implantable pump is configured to transfer fluid to and from the implantable reservoir and the body engaging implant respectively via a conduit wherein the implantable reservoir and the implantable electric motor are arranged externally to the implantable energized medical device.
11. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
an implantable electric motor arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit; wherein the implantable electric motor is connected to the implantable energy storage unit, the implantable electric motor being configured to operate the body engaging implant; wherein the implantable energy storage unit is arranged externally to the implantable energized medical device.
12. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
an implantable energy storage unit arranged in the first portion, the connecting portion or the second portion,
an implantable electric motor arranged in the first portion, the connecting portion or the second portion, the implantable electric motor being connected to the implantable energy storage unit, and
an implantable reservoir configured to hold a fluid, the implantable reservoir being arranged in the first portion, the connecting portion or the second portion,
wherein:
the first, second, and third planes are parallel to each other,
the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable pump configured to transfer fluid to and from the implantable reservoir and the body engaging implant respectively via a conduit; wherein the implantable electric motor is configured to operate the implantable pump; wherein the implantable pump is arranged externally to the implantable energized medical device.
13. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
   an implantable reservoir configured to hold a fluid arranged in the first portion, the connecting portion or the second portion,
   an implantable electric motor arranged in the first portion, the connecting portion or the second portion, and
   an implantable pump arranged in the first portion, the connecting portion or the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable energy storage unit, and
wherein the implantable pump is configured to transfer fluid to and from the implantable reservoir and the body engaging implant respectively via a conduit;
wherein the implantable electric motor is connected to the implantable energy storage unit and configured to operate the implantable pump;
wherein the implantable energy storage unit is arranged externally to the implantable energized medical device.
14. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
   an implantable reservoir configured to hold a fluid, the implantable reservoir being arranged in the first portion, the connecting portion or the second portion,
   an implantable pump arranged in the first portion, the connecting portion or the second portion, and
   an implantable energy storage unit arranged in the first portion, the connecting portion or the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable electric motor connected to the implantable energy storage unit, the implantable electric motor being configured to operate the implantable pump;
wherein the implantable pump is configured to transfer fluid to and from the implantable reservoir and the body engaging implant respectively via a conduit; and
wherein the implantable electric motor is arranged externally to the implantable energized medical device.
15. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
   an implantable pump arranged in the first portion, the connecting portion or the second portion,
   an implantable energy storage unit arranged in the first portion, the connecting portion or the second portion, and
   an electric motor arranged in the first portion, the connecting portion or the second portion,
   wherein:
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable reservoir configured to hold a fluid;
wherein the implantable electric motor is connected to the implantable energy storage unit, the implantable electric motor being configured to operate the implantable pump;
wherein the implantable pump is configured to transfer fluid to and from the implantable reservoir and the body engaging implant respectively via a conduit
wherein the implantable reservoir is arranged externally to the implantable energized medical device.
16. A system comprising
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion, and
   an implantable energy storage unit arranged in the first portion, the connecting portion or the second portion,
   wherein: wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes;
a body engaging implant configured to at least one of stretch, contract, expand, stimulate, and exert a force on body tissue or a body organ;
an implantable electric motor connected to the implantable energy storage unit, the implantable electric motor being configured to operate the body engaging implant;
wherein the implantable electric motor is arranged externally to the implantable energized medical device.
17. The system according to any one of aspects 1-16, wherein the implantable energized medical device further comprises a first wireless communication receiver configured to receive communication signals from outside the patient's body.
18. The system according to aspect 17, wherein the implantable energized medical device further comprises a second wireless communication transmitter arranged in the second portion, wherein the second wireless communication transmitter is configured to transmit communication signals to the first wireless communication receiver.
19. The system according to any one of the preceding aspects, wherein the implantable energized medical device further comprises a first wireless communication transmitter arranged in the first portion, the first wireless communication transmitter being configured to transmit communication signals outside of the patient's body.
20. The system according to aspect 19, wherein the implantable energized medical device further comprises a second wireless communication receiver arranged in the second portion, wherein the first wireless communication transmitter is configured to transmit communication signals to the second wireless communication receiver.
21. The system according to any one of the preceding aspects, wherein the implantable energized medical device further comprises a wireless energy receiver configured to receive energy transmitted wirelessly from outside the patient's body and deliver the received energy to the implantable energy storage unit.
22. The system according to any one of the preceding aspects, wherein the implantable energized medical device further comprises a control unit configured to control at least one of the body engaging implant, the implantable energy storage unit, the implantable pump, and the implantable electric motor.
23. The system according to any one of the preceding aspects, wherein the implantable electric motor is operatively connected to the implantable pump via a rotatable shaft.
24. The system according to any one of the preceding aspects, wherein the implantable electric motor is operatively connected to the implantable pump via a magnetic coupling.
25. The system according to any one of the preceding aspects, further comprising a gear arrangement arranged in the implantable energized medical device and operatively connected to the electric motor, the gear arrangement being configured to reduce the velocity and increase the force of movement generated by the electric motor.
26. The system according to any one of aspects 1-24, further comprising a gear arrangement arranged externally to the implantable energized medical device and operatively connected to the electric motor, the gear arrangement being configured to reduce the velocity and increase the force of movement generated by the electric motor.
27. The system according to any one of the preceding aspects, further comprising a housing configured to enclose at least the first portion, and wherein a first portion of the housing is made from titanium and a second portion of the housing is made from a ceramic material.
28. The system according to aspect 27, wherein the portion of the housing made from a ceramic material comprises at least one coil embedded in the ceramic material.
29. The system according to any one of the preceding aspects, further comprising a housing configured to enclose at least the second portion, and wherein a first portion of the housing is made from titanium and a second portion of the housing is made from a ceramic material.
30. The system according to aspect 29, wherein the portion of the housing made from a ceramic material comprises at least one coil embedded in the ceramic material.
31. The system according to any one of the preceding aspects, wherein the first portion is detachably connected to at least one of the second portion and the connecting portion.
32. The system according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
33. The system according to any one of the preceding aspects, wherein a connecting interface between the connecting portion and the second portion is excentric with respect to the second portion.
34. The system according to any one of the preceding aspects, wherein a connecting interface between the connecting portion and the first portion is excentric with respect to the first portion.
35. The system according to any one of the preceding aspects, wherein the second portion has a first end and a second end opposing the first end, wherein the second portion has a length between the first and second end.
36. The system according to aspect 35, wherein the first end and second end are separated in a direction parallel to the second plane.
37. The system according to aspect 35 or 36, wherein the first and second ends comprise an elliptical point respectively.
38. The system according to any one of aspects 35-37, wherein the first and second ends comprise a hemispherical end cap respectively.
39. The system according to any one of aspects 35-38, wherein the second portion has at least one circular cross-section along the length between the first and second end.
40. The system according to any one of aspects 35-39, wherein the second portion has at least one oval cross-section along the length between the first and second end.
41. The system according to any one of aspects 35-40, wherein the second portion has at least one elliptical cross-section along the length between the first and second end.
42. The system according to any one of the preceding aspects, further comprising a gear arrangement, wherein the gear arrangement is configured to reduce the velocity and increase the force of the movement generated by the electric motor.
43. The system according to aspect 42, wherein the gear arrangement is configured to transfer a force with a high velocity into a stronger force with lower velocity.
44. The system according to aspect 42 or 43, wherein the gear arrangement is configured to transfer a rotating force into a linear force.
45. The system according to any one of aspects 42-44, wherein the gear arrangement comprises a gear system.
46. The system according to any one of aspects 42-45, wherein the second portion comprises a magnetic coupling for transferring mechanical work from the electric motor through one of:
a barrier separating a first chamber of the second portion from a second chamber of the second portion,
a housing enclosing at least the second portion.
47. The system according to any one of the preceding aspects, wherein the pump is an hydraulic pump.
48. The system according to aspect 47, wherein the hydraulic pump comprises a pump comprising at least one compressible hydraulic reservoir.

### Aspect 438_Electro_Subcutaneous_Control_Pop-Rivet2_Same-Shape-A

1. An implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   the first portion is configured to be placed subcutaneously in the patient, and wherein the first portion comprises a connecting interface arrangement for transferring wired energy and/or wired communication signals and/or fluid to an additional implant in the patient.
2. The implantable energized medical device according to aspect 1, wherein a height of the first portion measured in a plane perpendicular to the first plane is 15 mm or less, such as 10 mm or less, such as 7 mm or less, such as 5 mm or less.
3. The implantable energized medical device according to aspect 1 or 2, wherein the connecting interface arrangement comprises a port for transferring fluid from the first portion to said additional implant.
4. The implantable energized medical device according to aspect 3, further comprising at least one conduit or tube for transferring said fluid, wherein the at least one conduit or tube is connected to the port.
5. The implantable energized medical device according to any one of the preceding aspects, further comprising at least one wire for energy and/or communication signals connected to the connecting interface arrangement.
6. The implantable energized medical device according to aspect 2, wherein the height of the first portion is a maximum height.
7. The implantable energized medical device according to any one of the preceding aspects, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
8. The implantable energized medical device according to aspect 7, wherein the first portion comprises a first energy storage unit connected to the first wireless energy receiver.
9. The implantable energized medical device according to any one of aspects 7 and 8, wherein the second portion comprises a second energy storage unit connected to the second wireless energy receiver.
10. The implantable energized medical device according to aspect 9, wherein at least one of the first and second energy storage unit is a solid-state battery.
11. The implantable energized medical device according to aspect 10, wherein the solid-state battery is a thionyl-chloride battery.
12. The implantable energized medical device according to any one of aspects 9-11, wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
13. The implantable energized medical device according to any one of aspects 7-12, wherein the first portion comprises a first controller comprising at least one processing unit.
14. The implantable energized medical device according to any one of aspects 7-13, wherein the second portion comprises a second controller comprising at least one processing unit.
15. The implantable energized medical device according to any one of aspects 13 and 14, wherein at least one of the first and second controller is connected to a wireless transceiver for communicating wirelessly with an external device.
16. The implantable energized medical device according to any one of aspects 13 and 14,
wherein: the first controller is connected to a first wireless communication receiver in the first portion for receiving wireless communication from an external device, the first controller is connected to a first wireless communication transmitter in the first portion for transmitting wireless communication to a second wireless communication receiver in the second portion.
17. The implantable energized medical device according to aspect 16, wherein the second controller is connected to the second wireless communication receiver for receiving wireless communication from the first portion.
18. The implantable energized medical device according to any one of aspects 7-17, wherein the first wireless energy receiver comprises a first coil and the wireless energy transmitter comprises a second coil.
19. The implantable energized medical device according to any one of aspects 7-18, wherein the first portion comprises a combined coil, wherein the combined coil is configured to receive energy wirelessly from an external wireless energy transmitter, and transmit energy wirelessly to the second wireless receiver of the second portion.
20. The implantable energized medical device according to any one of aspects 18 and 19, wherein at least one of the coils are embedded in a ceramic material.
21. The implantable energized medical device according to any one of the preceding aspects, further comprising a housing configured to enclose at least the first portion, and wherein a first portion of the housing is made from titanium and a second portion of the housing is made from a ceramic material.
22. The implantable energized medical device according to aspect 21, wherein the portion of the housing made from a ceramic material comprises at least one coil embedded in the ceramic material.
23. The implantable energized medical device according to any one of the preceding aspects, further comprising a housing configured to enclose at least the second portion, and wherein a first portion of the housing is made from titanium and a second portion of the housing is made from a ceramic material.
24. The implantable energized medical device according to aspect 23, wherein the portion of the housing made from a ceramic material comprises at least one coil embedded in the ceramic material.
25. The implantable energized medical device according to any one of the preceding aspects, wherein the first portion is detachably connected to at least one of the second portion and the connecting portion.
26. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
27. The implantable energized medical device according to any one of the preceding aspects, wherein a connecting interface between the connecting portion and the second portion is excentric with respect to the second portion.
28. The implantable energized medical device according to any one of the preceding aspects, wherein a connecting interface between the connecting portion and the first portion is excentric with respect to the first portion.
29. The implantable energized medical device according to any one of the preceding aspects, wherein the second portion has a first end and a second end opposing the first end, wherein the second portion has a length between the first and second end.
30. The implantable energized medical device according to aspect 29, wherein the first end and second end are separated in a direction parallel to the second plane.
31. The implantable energized medical device according to aspect 29 or 30, wherein the first and second ends comprise an elliptical point respectively.
32. The implantable energized medical device according to any one of aspects 29-31, wherein the first and second ends comprise a hemispherical end cap respectively.
33. The implantable energized medical device according to any one of aspects 29-32, wherein the second portion has at least one circular cross-section along the length between the first and second end.
34. The implantable energized medical device according to any one of aspects 29-33, wherein the second portion has at least one oval cross-section along the length between the first and second end.
35. The implantable energized medical device according to any one of aspects 29-34, wherein the second portion has at least one elliptical cross-section along the length between the first and second end.
36. The implantable energized medical device according to any one of the preceding aspects, further comprising a gear arrangement and an electric motor, wherein the gear arrangement is configured to reduce the velocity and increase the force of the movement generated by the electric motor.
37. The implantable energized medical device according to aspect 36, wherein the gear arrangement is configured to transfer a force with a high velocity into a stronger force with lower velocity.
38. The implantable energized medical device according to aspect 36 or 37, wherein the gear arrangement is configured to transfer a rotating force into a linear force.
39. The implantable energized medical device according to any one of aspects 36-38, wherein the gear arrangement comprises a gear system.
40. The implantable energized medical device according to any one of aspects 36-39, wherein the second portion comprises a magnetic coupling for transferring mechanical work from the electric motor through one of:
a barrier separating a first chamber of the second portion from a second chamber of the second portion,
a housing enclosing at least the second portion.
41. The implantable energized medical device according to any one of the preceding aspects, wherein the second portion comprises at least one hydraulic pump.
42. The implantable energized medical device according to aspect 41, wherein the hydraulic pump comprises a pump comprising at least one compressible hydraulic reservoir.

### Aspect 438_Electro_Subcutaneous_Control_Pop-Rivet2_Same-Shape-B

1. An implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   the first portion and the second portion are configured to be placed subcutaneously in the patient, such that the implantable energized medical device can be placed with either of the first portion and the second portion on the first side of the tissue portion.
2. The implantable energized medical device according to aspect 1, wherein a height of the second portion measured in a plane perpendicular to the second plane is 15 mm or less, such as 10 mm or less, such as 7 mm or less, such as 5 mm or less.
3. The implantable energized medical device according to aspect 1 or 2, wherein the first portion has a length in a plane parallel to the first plane, wherein the second portion has a length in a plane parallel to the second plane, and wherein the length of the first portion differ no more than 30% with regard to the length of the second portion, such as wherein the length of the first portion differ no more than 15% with regard to the length of the second portion, such as wherein the length of the first portion differ no more than 5% with regard to the length of the second portion, such as wherein the length of the first portion differ no more than 1% with regard to the length of the second portion.
4. The implantable energized medical device according to any one of the preceding aspects, wherein the first portion has a width in a plane parallel to the first plane, wherein the second portion has a width in a plane parallel to the second plane, and wherein the width of the first portion differ no more than 30% with regard to the width of the second portion, such as wherein the width of the first portion differ no more than 15% with regard to the width of the second portion, such as wherein the width of the first portion differ no more than 5% with regard to the width of the second portion, such as wherein the width of the first portion differ no more than 1% with regard to the width of the second portion.
5. The implantable energized medical device according to any one of the preceding aspects, wherein the first portion has a height in a plane perpendicular to the first plane, and wherein the height of the first portion differ no more than 30% with regard to the height of the second portion, such as wherein the height of the first portion differ no more than 15% with regard to the height of the second portion, such as wherein the height of the first portion differ no more than 5% with regard to the height of the second portion, such as wherein the height of the first portion differ no more than 1% with regard to the height of the second portion.
6. The implantable energized medical device according to any one of the preceding aspects, wherein a height of the first portion measured in a plane perpendicular to the first plane is 15 mm or less, such as 10 mm or less, such as 7 mm or less, such as 5 mm or less.
7. The implantable energized medical device according to any one of the preceding aspects, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
8. The implantable energized medical device according to aspect 7, wherein the first portion comprises a first energy storage unit connected to the first wireless energy receiver.
9. The implantable energized medical device according to any one of aspects 7 and 8, wherein the second portion comprises a second energy storage unit connected to the second wireless energy receiver.
10. The implantable energized medical device according to aspect 9, wherein at least one of the first and second energy storage unit is a solid-state battery.
11. The implantable energized medical device according to aspect 10, wherein the solid-state battery is a thionyl-chloride battery.
12. The implantable energized medical device according to any one of aspects 9-11, wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
13. The implantable energized medical device according to any one of aspects 7-12, wherein the first portion comprises a first controller comprising at least one processing unit.
14. The implantable energized medical device according to any one of aspects 7-13, wherein the second portion comprises a second controller comprising at least one processing unit.
15. The implantable energized medical device according to any one of aspects 13 and 14, wherein at least one of the first and second controller is connected to a wireless transceiver for communicating wirelessly with an external device.
16. The implantable energized medical device according to any one of aspects 13 and 14, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
17. The implantable energized medical device according to aspect 16, wherein the second controller is connected to the second wireless communication receiver for receiving wireless communication from the first portion.
18. The implantable energized medical device according to any one of aspects 7-17, wherein the first wireless energy receiver comprises a first coil and the wireless energy transmitter comprises a second coil.
19. The implantable energized medical device according to any one of aspects 7-18, wherein the first portion comprises a combined coil, wherein the combined coil is configured to receive energy wirelessly from an external wireless energy transmitter, and transmit energy wirelessly to the second wireless receiver of the second portion.
20. The implantable energized medical device according to any one of aspects 18 and 19, wherein at least one of the coils are embedded in a ceramic material.
21. The implantable energized medical device according to any one of the preceding aspects, further comprising a housing configured to enclose at least the first portion, and wherein a first portion of the housing is made from titanium and a second portion of the housing is made from a ceramic material.
22. The implantable energized medical device according to aspect 21, wherein the portion of the housing made from a ceramic material comprises at least one coil embedded in the ceramic material.
23. The implantable energized medical device according to any one of the preceding aspects, further comprising a housing configured to enclose at least the second portion, and wherein a first portion of the housing is made from titanium and a second portion of the housing is made from a ceramic material.
24. The implantable energized medical device according to aspect 23, wherein the portion of the housing made from a ceramic material comprises at least one coil embedded in the ceramic material.
25. The implantable energized medical device according to any one of the preceding aspects, wherein the first portion is detachably connected to at least one of the second portion and the connecting portion.
26. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
27. The implantable energized medical device according to any one of the preceding aspects, wherein a connecting interface between the connecting portion and the second portion is excentric with respect to the second portion.
28. The implantable energized medical device according to any one of the preceding aspects, wherein a connecting interface between the connecting portion and the first portion is excentric with respect to the first portion.
29. The implantable energized medical device according to any one of the preceding aspects, wherein the second portion has a first end and a second end opposing the first end, wherein the second portion has a length between the first and second end.
30. The implantable energized medical device according to aspect 29, wherein the first end and second end are separated in a direction parallel to the second plane.
31. The implantable energized medical device according to aspect 29 or 30, wherein the first and second ends comprise an elliptical point respectively.
32. The implantable energized medical device according to any one of aspects 29-31, wherein the first and second ends comprise a hemispherical end cap respectively.
33. The implantable energized medical device according to any one of aspects 29-32, wherein the second portion has at least one circular cross-section along the length between the first and second end.
34. The implantable energized medical device according to any one of aspects 29-33, wherein the second portion has at least one oval cross-section along the length between the first and second end.
35. The implantable energized medical device according to any one of aspects 29-34, wherein the second portion has at least one elliptical cross-section along the length between the first and second end.
36. The implantable energized medical device according to any one of the preceding aspects, further comprising a gear arrangement and an electric motor, wherein the gear arrangement is configured to reduce the velocity and increase the force of the movement generated by the electric motor.
37. The implantable energized medical device according to aspect 36, wherein the gear arrangement is configured to transfer a force with a high velocity into a stronger force with lower velocity.
38. The implantable energized medical device according to aspect 36 or 37, wherein the gear arrangement is configured to transfer a rotating force into a linear force.
39. The implantable energized medical device according to any one of aspects 36-38, wherein the gear arrangement comprises a gear system.
40. The implantable energized medical device according to any one of aspects 36-39, wherein the second portion comprises a magnetic coupling for transferring mechanical work from the electric motor through one of:
a barrier separating a first chamber of the second portion from a second chamber of the second portion,
a housing enclosing at least the second portion.
41. The implantable energized medical device according to any one of the preceding aspects, wherein the second portion comprises at least one hydraulic pump.
42. The implantable energized medical device according to aspect 41, wherein the hydraulic pump comprises a pump comprising at least one compressible hydraulic reservoir.

### Aspect 439_Electro_Subcutaneous_Control_Pop-Rivet2_First-Portion-Polymer

1. An implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion,
   a connecting portion configured to be placed through a hole in the tissue portion extending between the first and second sides of the tissue portion, the connecting portion having a third cross-sectional area in a third plane and being configured to connect the first portion to the second portion,
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes,
   the second portion is hermetically sealed by means of an outer wall of the second portion comprising titanium.
2. The implantable energized medical device according to aspect 1, wherein the first portion comprises an outer wall comprising a polymer material.
3. The implantable energized medical device according to aspect 2, wherein the outer wall of the first portion consists of the polymer material.
4. The implantable energized medical device according to any one of the preceding aspects, wherein the second portion is hermetically sealed with respect to the connecting portion and the first portion.
5. The implantable energized medical device according to any one of the preceding aspects, wherein the outer wall of the second portion comprises a ceramic portion integrated in, or brazed to, the titanium.
6. The implantable energized medical device according to aspect 5, wherein the ceramic portion of the second portion comprises at least one metallic lead travelling through the ceramic portion for transferring electrical energy or information from within the second portion to an outside of the second portion and/or from the outside of the second portion to an inside of the second portion.
7. The implantable energized medical device according to aspect 5 or 6, wherein the at least one metallic lead is integrated in, or brazed to, the ceramic portion of the second portion, such that the at least one metallic lead can pass said ceramic portion without being further insulated.
8. The implantable energized medical device according to any one of aspects 5 to 7, wherein the connecting portion comprises an outer wall comprising titanium.
9. The implantable energized medical device according to aspect 8, wherein the outer wall of the connecting portion comprises a ceramic portion integrated in, or brazed to, the titanium.
10. The implantable energized medical device according to aspect 9, wherein the ceramic portion of the connecting portion comprises at least one metallic lead travelling through said ceramic portion for transferring electrical energy or information from within the connecting portion to an outside of the connecting portion and/or from the outside of the connecting portion to an inside of the connecting portion.
11. The implantable energized medical device according to aspect 9 or 10, wherein the at least one metallic lead is integrated in, or brazed to, the ceramic portion of the connecting portion, such that the at least one metallic lead can pass said ceramic portion without being further insulated.
12. The implantable energized medical device according to any one of the preceding aspects, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
13. The implantable energized medical device according to aspect 12, wherein the first portion comprises a first energy storage unit connected to the first wireless energy receiver.
14. The implantable energized medical device according to any one of aspects 12 and 13, wherein the second portion comprises a second energy storage unit connected to the second wireless energy receiver.
15. The implantable energized medical device according to aspect 14, wherein at least one of the first and second energy storage unit is a solid-state battery.
16. The implantable energized medical device according to aspect 15, wherein the solid-state battery is a thionyl-chloride battery.
17. The implantable energized medical device according to any one of aspects 14-16,
wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
18. The implantable energized medical device according to any one of aspects 12-17, wherein the first portion comprises a first controller comprising at least one processing unit.
19. The implantable energized medical device according to any one of aspects 12-18, wherein the second portion comprises a second controller comprising at least one processing unit.
20. The implantable energized medical device according to any one of aspects 18 and 19, wherein at least one of the first and second controller is connected to a wireless transceiver for communicating wirelessly with an external device.
21. The implantable energized medical device according to any one of aspects 18 and 19, wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
22. The implantable energized medical device according to aspect 21, wherein the second controller is connected to the second wireless communication receiver for receiving wireless communication from the first portion.
23. The implantable energized medical device according to any one of aspects 12-22, wherein the first wireless energy receiver comprises a first coil and the wireless energy transmitter comprises a second coil.
24. The implantable energized medical device according to any one of aspects 12-23, wherein the first portion comprises a combined coil, wherein the combined coil is configured to receive energy wirelessly from an external wireless energy transmitter, and transmit energy wirelessly to the second wireless receiver of the second portion.
25. The implantable energized medical device according to any one of aspects 23 and 24, wherein at least one of the coils are embedded in a ceramic material.
26. The implantable energized medical device according to any one of the preceding aspects, wherein the first portion is detachably connected to at least one of the second portion and the connecting portion.
27. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
28. The implantable energized medical device according to any one of the preceding aspects, wherein a connecting interface between the connecting portion and the second portion is excentric with respect to the second portion.
29. The implantable energized medical device according to any one of the preceding aspects, wherein a connecting interface between the connecting portion and the first portion is excentric with respect to the first portion.
30. The implantable energized medical device according to any one of the preceding aspects, wherein the second portion has a first end and a second end opposing the first end, wherein the second portion has a length between the first and second end.
31. The implantable energized medical device according to aspect 30, wherein the first end and second end are separated in a direction parallel to the second plane.
32. The implantable energized medical device according to aspect 30 or 31, wherein the first and second ends comprise an elliptical point respectively.
33. The implantable energized medical device according to any one of aspects 30-32, wherein the first and second ends comprise a hemispherical end cap respectively.
34. The implantable energized medical device according to any one of aspects 30-33, wherein the second portion has at least one circular cross-section along the length between the first and second end.
35. The implantable energized medical device according to any one of aspects 30-34, wherein the second portion has at least one oval cross-section along the length between the first and second end.
36. The implantable energized medical device according to any one of aspects 30-35, wherein the second portion has at least one elliptical cross-section along the length between the first and second end.
37. The implantable energized medical device according to any one of the preceding aspects, further comprising a gear arrangement and an electric motor, wherein the gear arrangement is configured to reduce the velocity and increase the force of the movement generated by the electric motor.
38. The implantable energized medical device according to aspect 37, wherein the gear arrangement is configured to transfer a force with a high velocity into a stronger force with lower velocity.
39. The implantable energized medical device according to aspect 37 or 38, wherein the gear arrangement is configured to transfer a rotating force into a linear force.
40. The implantable energized medical device according to any one of aspects 37-39, wherein the gear arrangement comprises a gear system.
41. The implantable energized medical device according to any one of aspects 37-40, wherein the second portion comprises a magnetic coupling for transferring mechanical work from the electric motor through one of
a barrier separating a first chamber of the second portion from a second chamber of the second portion,
a housing enclosing at least the second portion.
42. The implantable energized medical device according to any one of the preceding aspects, wherein the second portion comprises at least one hydraulic pump.
43. The implantable energized medical device according to aspect 42, wherein the hydraulic pump comprises a pump comprising at least one compressible hydraulic reservoir.

### Aspect 448_Electro_Subcutaneous_Control_Pop-Rivet2_Second-Portion-Reservoir

1. An implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and being configured to connect the first portion to the second portion,
   wherein the second portion comprises or forms a reservoir for holding a fluid;
   the implantable energized medical device further comprising:
   a sealed container configured to protrude into the reservoir;
   an actuator connected to the sealed container, the actuator being configured to expand or retract the sealed container to change the volume of the sealed container for pumping fluid to or from the reservoir;
   wherein:
   the first, second, and third planes are parallel to each other,
   the third cross-sectional area is smaller than the first and second cross-sectional areas, such that the first portion and second portion are prevented from travelling through the hole in the tissue portion in a direction perpendicular to the first, second and third planes.
2. The implantable energized medical device according to aspect 1, wherein the actuator comprises an electric motor.
3. The implantable energized medical device according to aspect 1 or 2, wherein the actuator is arranged in the connecting portion.
4. The implantable energized medical device according to any one of the preceding aspects, wherein the actuator is partly or fully arranged inside the sealed container.
5. The implantable energized medical device according to any one of the preceding aspects, wherein the second portion comprises a port in fluid communication with the reservoir for transferring fluid between the reservoir and an additional implant in the patient.
6. The implantable energized medical device according to aspect 5, further comprising a conduit connected to the port, the conduit being configured to transfer fluid between the reservoir and the additional implant.
7. The implantable energized medical device according to any one of the preceding aspects, further comprising an injection port for introducing fluid, the injection port being arranged in the first portion.
8. The implantable energized medical device according to aspect 7, further comprising an internal conduit connecting the injection port to the reservoir.
9. The implantable energized medical device according to any one of the preceding aspects, wherein the sealed container is a bellows.
10. The implantable energized medical device according to aspect 9, wherein the bellows is a metallic bellows.
11. The implantable energized medical device according to any one of the preceding aspects, wherein at least a portion of the sealed container configured to be in contact with fluid comprises metal.
12. The implantable energized medical device according to any one of the preceding aspects, wherein the volume of the sealed container can be altered such that the volume of the sealed container is more than 60% of the maximum volume of the reservoir.
13. The implantable energized medical device according to any one of the preceding aspects, wherein the sealed container comprises at least one flexible portion, and wherein the flexible portion enable at least one of compression and expansion of the sealed container.
14. The implantable energized medical device according to any one of the preceding aspects, wherein the sealed container comprises at least one elastic portion, and wherein the elastic portion enable at least one of compression and expansion of the sealed container.
18. The implantable energized medical device according to any one of the preceding aspects, further comprising a first energy storage unit and/or a second energy storage unit for powering the actuator.
19. The implantable energized medical device according to aspect 18, wherein
the first portion comprises a first wireless energy receiver for receiving energy transmitted wirelessly by an external wireless energy transmitter, and an internal wireless energy transmitter configured to transmit energy wirelessly to the second portion, and
the second portion comprises a second wireless energy receiver configured to receive energy transmitted wirelessly by the internal wireless energy transmitter.
21. The implantable energized medical device according to aspect 19, wherein the first energy storage unit is connected to the first wireless energy receiver.
22. The implantable energized medical device according to aspect 19 or 20, wherein the second portion comprises the second energy storage unit, wherein the second energy storage unit is connected to the second wireless energy receiver.
23. The implantable energized medical device according to aspect 21, wherein at least one of the first and second energy storage unit is a solid-state battery.
24. The implantable energized medical device according to aspect 22, wherein the solid-state battery is a thionyl-chloride battery.
25. The implantable energized medical device according to any one of aspects 22-24,
wherein:
the first wireless energy receiver is configured to receive energy transmitted wirelessly by the external wireless energy transmitter and store the received energy in the first energy storage unit,
the internal wireless energy transmitter is configured to wirelessly transmit energy stored in the first energy storage unit to the second wireless energy receiver, and
the second wireless energy receiver is configured to receive energy transmitted wirelessly by the internal wireless energy transmitter and store the received energy in the second energy storage unit.
26. The implantable energized medical device according to any one of the preceding aspects, wherein the first portion comprises a first controller comprising at least one processing unit.
27. The implantable energized medical device according to any one of the preceding aspects, wherein the second portion comprises a second controller comprising at least one processing unit.
28. The implantable energized medical device according to aspect 26 or 27, wherein the first controller and/or the second controller is configured to control the actuator.
29. The implantable energized medical device according to any one of aspects 26-28, wherein at least one of the first and second controller is connected to a wireless transceiver for communicating wirelessly with an external device.
30. The implantable energized medical device according to any one of aspects 26-28,
wherein:
the first controller is connected to a first wireless communication receiver in the first portion for receiving wireless communication from an external device,
the first controller is connected to a first wireless communication transmitter in the first portion for transmitting wireless communication to a second wireless communication receiver in the second portion.
31. The implantable energized medical device according to aspect 30, wherein the second controller is connected to the second wireless communication receiver for receiving wireless communication from the first portion.
32. The implantable energized medical device according to any one of aspects 19-31, wherein the first wireless energy receiver comprises a first coil and the wireless energy transmitter comprises a second coil.
33. The implantable energized medical device according to any one of aspects 19-32, wherein the first portion comprises a combined coil, wherein the combined coil is configured to receive energy wirelessly from an external wireless energy transmitter, and transmit energy wirelessly to the second wireless receiver of the second portion.
34. The implantable energized medical device according to any one of aspects 32 and 33, wherein at least one of the coils are embedded in a ceramic material.
35. The implantable energized medical device according to any one of the preceding aspects, further comprising a housing configured to enclose at least the first portion, and wherein a first portion of the housing is made from titanium and a second portion of the housing is made from a ceramic material.
36. The implantable energized medical device according to aspect 35, wherein the portion of the housing made from a ceramic material comprises at least one coil embedded in the ceramic material.
37. The implantable energized medical device according to any one of the preceding aspects, further comprising a housing configured to enclose at least the second portion, and wherein a first portion of the housing is made from titanium and a second portion of the housing is made from a ceramic material.
38. The implantable energized medical device according to aspect 37, wherein the portion of the housing made from a ceramic material comprises at least one coil embedded in the ceramic material.
39. The implantable energized medical device according to any one of the preceding aspects, wherein the first portion is detachably connected to at least one of the second portion and the connecting portion.
40. The implantable energized medical device according to any one of the preceding aspects, wherein the connecting portion comprising a flange having a flange area being larger than a cross-section area of the hole in the tissue portion, such that the flange is hindered from travelling through the hole in the tissue portion, such that the second portion and the connecting portion can be held in position by the tissue portion of the patient also when the first portion is disconnected from the connecting portion.
41. The implantable energized medical device according to any one of the preceding aspects, wherein a connecting interface between the connecting portion and the second portion is excentric with respect to the second portion.
42. The implantable energized medical device according to any one of the preceding aspects, wherein a connecting interface between the connecting portion and the first portion is excentric with respect to the first portion.
43. The implantable energized medical device according to any one of the preceding aspects, wherein the second portion has a first end and a second end opposing the first end, wherein the second portion has a length between the first and second end.
44. The implantable energized medical device according to aspect 43, wherein the first end and second end are separated in a direction parallel to the second plane.
45. The implantable energized medical device according to aspect 43 or 44, wherein the first and second ends comprise an elliptical point respectively.
46. The implantable energized medical device according to any one of aspects 43-45, wherein the first and second ends comprise a hemispherical end cap respectively.
47. The implantable energized medical device according to any one of aspects 43-46, wherein the second portion has at least one circular cross-section along the length between the first and second end.
48. The implantable energized medical device according to any one of aspects 43-47, wherein the second portion has at least one oval cross-section along the length between the first and second end.
49. The implantable energized medical device according to any one of aspects 43-48, wherein the second portion has at least one elliptical cross-section along the length between the first and second end.
50. The implantable energized medical device according to aspect 2, further comprising a gear arrangement, wherein the gear arrangement is configured to reduce the velocity and increase the force of the movement generated by the electric motor.
51. The implantable energized medical device according to aspect 50, wherein the gear arrangement is configured to transfer a force with a high velocity into a stronger force with lower velocity.
52. The implantable energized medical device according to aspect 50 or 51, wherein the gear arrangement is configured to transfer a rotating force into a linear force.
53. The implantable energized medical device according to any one of aspects 50-52, wherein the gear arrangement comprises a gear system.
54. The implantable energized medical device according to any one of aspects 50-53, wherein the second portion comprises a magnetic coupling for transferring mechanical work from the electric motor through one of:
a barrier separating a first chamber of the second portion from a second chamber of the second portion,
a housing enclosing at least the second portion.

### Aspect 454 Dual Remote Controls

1. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user.
2. The communication system according to embodiment 1, wherein the first remote control comprises an input device for receiving a first user input, and wherein the first remote control is configured to transmit the first user input to the implantable medical device.
3. The communication system according to any one of the preceding embodiments, wherein the second remote control is configured to receive second user input from the patient display device and to transmit the second user input to the implantable medical device.
4. The communication system according to any one of the preceding embodiments, wherein the data comprises a control command for the medical device.
5. The communication system according to any one of the preceding embodiments, wherein at least one of the first wireless communication unit and the second wireless communication unit is configured to send or receive data using near-field magnetic induction.
6. The communication system according to embodiment 5, wherein at least one of the first wireless communication unit and the second wireless communication unit comprises a transmitter coil for modulating a magnetic field for transmitting the data, and wherein the implantable medical device comprises a receiving coil and an NFMI receiver connected to the receiving coil to receive the data.
7. The communication system according to embodiment 6, wherein the transmitter coil is configured to modulate a magnetic field, and the NFMI receiver is adapted to measure the magnetic field in the receiving coil.
8. The communication system according to any one of the preceding embodiments, wherein at least one of the first wireless communication unit and the second wireless communication unit is configured to wirelessly charge the medical device using near-field magnetic induction.
9. The communication system according to embodiment 9, wherein the medical device comprises a coil for receiving wireless energy for charging the implant via near-field magnetic induction.
10. The communication system according to any one of the preceding embodiments, wherein the second and third communication units are configured to transmit and/or receive data using different network protocols.
11. The communication system according to any one of the preceding embodiments, wherein the second and third communication units are configured to transmit and/or receive data using different frequency bands.
12. The communication system according to any one of the preceding embodiments, wherein at least one of the first remote control, the second remote control and the implantable medical device comprises a Bluetooth transceiver.
13. The communication system according to any one of the preceding embodiments, wherein at least one of first remote control, the second remote control and the implantable medical device comprises a UWB transceiver.
14. The communication system according to embodiment 10, wherein the network protocol is one from the list of: Radio Frequency type protocol, RFID type protocol, WLAN type protocol,
Bluetooth type protocol, BLE type protocol, NFC type protocol, 3G/4G/5G type protocol, and GSM type protocol.
15. The communication system according to any one of the preceding embodiments, wherein the second communication unit has a longer effective range than the third communication unit.
16. The communication system according to any one of the preceding embodiments, wherein the second remote control is configured to communicate with a consumer electronics device.
17. The communication system according to embodiment 16, wherein the patient display device comprises the consumer electronics device.
18. The communication system according to any one of the preceding embodiments, wherein the first remote control is configured to control functions of the implantable medical device based on user input to the first remote control.

### Aspect 454B Dual Remote Controls

1. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a drug delivery device for injecting a drug into a patient's body such as stimulation of penis erection wherein
the drug is injected into the patient's body into at least one of
both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated,
characterized by an energy transmission device for wireless transmission of energy from outside the patient's body to inside the patient's body for use in connection with the operation of the prosthesis, when the prosthesis is implanted.
12. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises a system for treating a female patient to avoid or promote pregnancy comprising a restriction device adapted to postoperatively restrict and release an oviduct of the patient.
18. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an obesity treatment device comprising:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
27. The communication system according to embodiment 1-26, wherein the first remote control comprises an input device for receiving a first user input, and wherein the first remote control is configured to transmit the first user input to the implantable medical device.
28. The communication system according to any one of the preceding embodiments, wherein the second remote control is configured to receive second user input from the patient display device and to transmit the second user input to the implantable medical device.
29. The communication system according to any one of the preceding embodiments, wherein the data comprises a control command for the medical device.
30. The communication system according to any one of the preceding embodiments, wherein at least one of the first wireless communication unit and the second wireless communication unit is configured to send or receive data using near-field magnetic induction.
31. The communication system according to embodiment 30, wherein at least one of the first wireless communication unit and the second wireless communication unit comprises a transmitter coil for modulating a magnetic field for transmitting the data, and wherein the implantable medical device comprises a receiving coil and an NFMI receiver connected to the receiving coil to receive the data.
32. The communication system according to embodiment 31, wherein the transmitter coil is configured to modulate a magnetic field, and the NFMI receiver is adapted to measure the magnetic field in the receiving coil.
33. The communication system according to any one of the preceding embodiments, wherein at least one of the first wireless communication unit and the second wireless communication unit is configured to wirelessly charge the medical device using near-field magnetic induction.
34. The communication system according to embodiment 33, wherein the medical device comprises a coil for receiving wireless energy for charging the implant via near-field magnetic induction.
35. The communication system according to any one of the preceding embodiments, wherein the second and third communication units are configured to transmit and/or receive data using different network protocols.
36. The communication system according to any one of the preceding embodiments, wherein the second and third communication units are configured to transmit and/or receive data using different frequency bands.
37. The communication system according to any one of the preceding embodiments, wherein at least one of the first remote control, the second remote control and the implantable medical device comprises a Bluetooth transceiver.
38. The communication system according to any one of the preceding embodiments, wherein at least one of first remote control, the second remote control and the implantable medical device comprises a UWB transceiver.
39. The communication system according to embodiment 38, wherein the network protocol is one from the list of: Radio Frequency type protocol, RFID type protocol, WLAN type protocol,
Bluetooth type protocol, BLE type protocol, NFC type protocol, 3G/4G/5G type protocol, and GSM type protocol.
40. The communication system according to any one of the preceding embodiments, wherein the second communication unit has a longer effective range than the third communication unit.
41. The communication system according to any one of the preceding embodiments, wherein the second remote control is configured to communicate with a consumer electronics device.
42. The communication system according to embodiment 41, wherein the patient display device comprises the consumer electronics device.
43. The communication system according to any one of the preceding embodiments, wherein the first remote control is configured to control functions of the implantable medical device based on user input to the first remote control.

### Aspect 457 Controlling Energy Transfer Accumulated/PID

1. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal.
2. The method according to embodiment 1, wherein determining an accumulated amount of received energy is determined by the internal energy receiver.
3. The method according to any one of the preceding embodiments, wherein determining a current change is performed by the internal energy receiver.
4. The method according to any one of the preceding embodiments, wherein the internal energy received comprises a PID regulator for controlling the energy transfer.
5. The method according to embodiment 4, wherein the PID regulator is implemented in a microcontroller.
6. The method according to any one of the preceding embodiments, wherein determining a control signal is performed by the internal energy receiver.
7. The method according to embodiment 6, wherein the control signal is transmitted to the external energy source, and wherein the external energy source is configured to adjust the transmitted energy based on the control signal.
8. The method according to any one of the preceding embodiments, wherein controlling the energy transfer is controlled by the internal energy receiver.
9. The method according to any one of the preceding embodiments, wherein controlling the energy transfer is performed by the external energy source.
10. The method according to any one of the preceding embodiments, wherein controlling the energy transfer comprises adjusting the energy transfer efficiency.
11. The method according any one of the preceding embodiments, wherein the external device comprises a transmitter coil for modulating a magnetic field for transmitting data or transmitting energy, and
wherein the implantable medical device comprises a receiving coil and an NFMI receiver connected to the receiving coil to receive the data or the energy.
12. The method according to any one of the preceding embodiments, wherein at least one of the first wireless communication unit and the second wireless communication unit is configured to wirelessly charge the implantable medical device using near-field magnetic induction.
13. The method according to embodiment 9, wherein the implantable medical device comprises a coil for receiving wireless energy for charging the implant via near-field magnetic induction.
14. The method according to any one of the preceding embodiments, further comprising: receiving energy in pulses according to a pulse pattern, and measuring the received pulse pattern.
15. The method according to embodiment 15, further comprising: determining that the pulse pattern deviates from a predefined pulse pattern, and controlling the energy transfer based on the determination.
16. The method according to any one of the preceding embodiments, further comprising: measuring a temperature in the implantable medical device or in the body of the patient, and controlling the energy transfer in response to the measured temperature.
17. The method according to any one of the preceding embodiments, wherein the implantable medical device comprises at least one coil connected to a variable impedance, the method further comprising controlling the energy transfer by controlling the variable impedance.
18. The method according to any one of the preceding embodiments, wherein the implantable medical device comprises at least one coil having a plurality of windings, wherein the plurality of windings each are connected to a respective variable impedance, the method further comprising controlling the energy transfer by controlling the respective variable impedance individually.

### Aspect 457B Controlling Energy Transfer Accumulated/PID

1. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion,
wherein:
the first, second, third and fourth planes are parallel to each other,
the third cross-sectional area is smaller than the first, second and fourth cross-sectional areas, such that the first portion, second portion and connecting portion are prevented from travelling through the hole in the tissue portion in a direction perpendicular to the first, second and third planes, and
the first portion is detachably connected to at least one of the connecting portion and the second portion.
2. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: a system for treating a female patient to avoid or promote pregnancy comprising a restriction device adapted to postoperatively restrict and release an oviduct of the patient.
18. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an apparatus for treating obesity of an patient having a stomach with a food cavity, the apparatus comprising:
a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
at least one adjustable stretching device adapted to be at least substantially invaginated by a stomach wall portion of the patient with the outer surface of the stretching device resting against the stomach wall and adapted to stretch a portion of stomach wall, and
a fluid connection device interconnecting the volume filling device and the stretching device.
23. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises: an obesity treatment device comprising:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part,
wherein:
the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
27. The method according to any one of the preceding embodiments, wherein determining an accumulated amount of received energy is determined by the internal energy receiver.
28. The method according to any one of the preceding embodiments, wherein determining a current change is performed by the internal energy receiver.
29. The method according to any one of the preceding embodiments, wherein the internal energy received comprises a PID regulator for controlling the energy transfer.
30. The method according to embodiment 29, wherein the PID regulator is implemented in a microcontroller.
31. The method according to any one of the preceding embodiments, wherein determining a control signal is performed by the internal energy receiver.
32. The method according to embodiment 31, wherein the control signal is transmitted to the external energy source, and wherein the external energy source is configured to adjust the transmitted energy based on the control signal.
33. The method according to any one of the preceding embodiments, wherein controlling the energy transfer is controlled by the internal energy receiver.
34. The method according to any one of the preceding embodiments, wherein controlling the energy transfer is performed by the external energy source.
35. The method according to any one of the preceding embodiments, wherein controlling the energy transfer comprises adjusting the energy transfer efficiency.
36. The method according any one of the preceding embodiments, wherein the external device comprises a transmitter coil for modulating a magnetic field for transmitting data or transmitting energy, and
wherein the implantable medical device comprises a receiving coil and an NFMI receiver connected to the receiving coil to receive the data or the energy.
37. The method according to any one of the preceding embodiments, wherein at least one of the first wireless communication unit and the second wireless communication unit is configured to wirelessly charge the implantable medical device using near-field magnetic induction.
38. The method according to embodiment 37, wherein the implantable medical device comprises a coil for receiving wireless energy for charging the implant via near-field magnetic induction.
39. The method according to any one of the preceding embodiments, further comprising: receiving energy in pulses according to a pulse pattern, and measuring the received pulse pattern.
40. The method according to embodiment 39, further comprising: determining that the pulse pattern deviates from a predefined pulse pattern, and controlling the energy transfer based on the determination.
41. The method according to any one of the preceding embodiments, further comprising: measuring a temperature in the implantable medical device or in the body of the patient, and controlling the energy transfer in response to the measured temperature.
42. The method according to any one of the preceding embodiments, wherein the implantable medical device comprises at least one coil connected to a variable impedance, the method further comprising controlling the energy transfer by controlling the variable impedance.
43. The method according to any one of the preceding embodiments, wherein the implantable medical device comprises at least one coil having a plurality of windings, wherein the plurality of windings each are connected to a respective variable impedance, the method further comprising controlling the energy transfer by controlling the respective variable impedance individually.

### Aspect 453 Voice Control

1. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant.
2. The method according to embodiment 1, wherein adjusting the amplitude comprises at least one of: filtering, cancelling and amplifying the at least one frequency.
3. The method according to any one of the preceding embodiments, wherein at least one of the first and second audio training phrase is a spoken audio training phrase.
4. The method according to embodiment 3, wherein the spoken audio training phrase is spoken by the patient the implant is implanted in.
5. The method according to any one of the preceding embodiments, wherein the first audio training phrase comprises the at least one voice command related to an instruction for the control of the medical implant.
6. The method according to any one of the preceding embodiments, wherein the first and second audio training phrases is the same voice command.
7. The method according to any one of the preceding embodiments, wherein the first and second audio training phrases are different.
8. The method according to any one of the preceding embodiments, wherein creating the transfer function comprises amplifying frequencies muffled by the location of the medical implant in the body of the patient.
9. The method according to any one of the preceding embodiments, wherein creating the transfer function comprises filtering or cancelling noise generated by the body.
10. The method according to any one of the preceding embodiments, wherein the medical implant is configured to receive voice commands related to an instruction for control of the medical implant.
11. The method according to any one of the preceding embodiments, wherein the voice command relates to at least one of
   performing a function of the medical device;
   using a sensor to measure a parameter relating to a condition of the patient or a condition of the medial implant;
   sending or receiving data from the medical implant.
12. A method of using a voice command to control a medical implant, wherein the method comprises:
   receiving an audio command phrase for the medical device;
   applying a transfer function to create an enhanced audio command phrase;
   determining a corresponding command for the medical based on the enhance audio command phrase; and
   sending the command to the medical device.
13. The method according to embodiment 12, further comprising executing, by the medical device, the command.

### Aspect 453B Voice Control

1. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising:
a drug delivery device for injecting a drug into a patient's body such as stimulation of penis erection wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant, the medical implant comprising: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: a system for treating a female patient to avoid or promote pregnancy comprising a restriction device adapted to postoperatively restrict and release an oviduct of the patient.
18. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
   an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
a device for treating obesity of a patient, the device comprising:
   at least one operable stretching device implantable in the patient and adapted to stretch a portion of the patient's stomach wall, and
   an implantable control unit for automatically controlling the operable stretching device, when the control unit and stretching device are implanted, to stretch the stomach wall portion in connection with the patient eating such that satiety is created,
   wherein the stretching device comprises a first and a second engaging part, wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
24. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising: an obesity treatment device comprising:
   at least one operable stretching device implantable in a patient and adapted to stretch a portion of the patient's stomach wall, and
   an implantable operation device for operating the stretching device, when implanted, to stretch the stomach wall portion such that satiety is created,
   wherein the operable stretching device is adapted to be placed against the outside of the stomach wall.
25. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
26. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
the medical implant comprising:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
27. The method according to any one of the preceding embodiments, wherein adjusting the amplitude comprises at least one of filtering, cancelling and amplifying the at least one frequency.
28. The method according to any one of the preceding embodiments, wherein at least one of the first and second audio training phrase is a spoken audio training phrase.
29. The method according to embodiment 28, wherein the spoken audio training phrase is spoken by the patient the implant is implanted in.
30. The method according to any one of the preceding embodiments, wherein the first audio training phrase comprises the at least one voice command related to an instruction for the control of the medical implant.
31. The method according to any one of the preceding embodiments, wherein the first and second audio training phrases is the same voice command.
32. The method according to any one of the preceding embodiments, wherein the first and second audio training phrases are different.
33. The method according to any one of the preceding embodiments, wherein creating the transfer function comprises amplifying frequencies muffled by the location of the medical implant in the body of the patient.
34. The method according to any one of the preceding embodiments, wherein creating the transfer function comprises filtering or cancelling noise generated by the body.
35. The method according to any one of the preceding embodiments, wherein the medical implant is configured to receive voice commands related to an instruction for control of the medical implant.
36. The method according to any one of the preceding embodiments, wherein the voice command relates to at least one of:
   performing a function of the medical device;
   using a sensor to measure a parameter relating to a condition of the patient or a condition of the medial implant;
   sending or receiving data from the medical implant.

### Aspect Large Coil

1. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil.
2. The system according to any one of the preceding embodiments, wherein the system further comprises:
an internal controller connected to the internal energy receiver, for controlling the amount of energy received by the internal energy receiver.
3. The system according to any one of the preceding embodiments, wherein the internal energy receiver further comprises a measurement unit for measuring a parameter related to the implantable medical device or the body of the patient.
4. The system according to any one of the preceding embodiments, wherein the controller is configured to measure the accumulated energy received by the internal energy receiver over a period of time and to measure a current change in energy received, and to control the energy received based on the accumulated energy and the current change.
5. The system according to any one of the preceding embodiments, wherein the controlled comprises a Proportional-Integral-Derivative, PID, regulator for controlling the received energy.
6. The system according to any one of the preceding embodiments, wherein the internal energy received comprises a variable impedance.
7. The system according to embodiment 6, when depending on any one of embodiments 2-5, wherein the internal energy receiver is configured to control the resonant frequency by controlling the variable impedance.
8. The system according to embodiment 7, wherein the controller is configured to vary the variable impedance in response to a measured parameter deviating from a predetermined interval or exceeding a threshold value.
9. The system according to embodiment 7, wherein the parameter relates to the energy received by the coil over a time period.
10. The system according to embodiment 8 or 9, wherein the measurement unit is configured to measure a parameter related to a change in energy received by the coil.
11. The system according to embodiment 3-10, wherein the receiving unit is configured to receive transferred energy in pulses according to a pulse pattern, and wherein the measurement unit is configured to measure a parameter related to the pulse pattern.
12. The system according to embodiment 3-10, wherein the receiving unit is configured to receive transferred energy in pulses according to a pulse pattern, and wherein the measurement unit is configured to measure a parameter related to the pulse pattern.
13. The system according to embodiment 3-11, wherein the controller is configured to control the variable impedance in response to the pulse pattern deviating from a predefined pulse pattern.
14. The system according to embodiment 6-13, wherein:
the variable impedance comprises a resistor and a capacitor,
the variable impedance comprises a resistor and an inductor,
the variable impedance comprises an inductor and a capacitor,
the variable impedance comprises a digitally tuned capacitor,
the variable impedance comprises a digital potentiometer, or
the variable impedance comprises a variable inductor.
15. The system according any one of the preceding embodiments, wherein the diameter of the primary coil is more than 0.5 cm.
16. The system according to embodiment 15, wherein the diameter of the primary coil is more than
10 cm.
17. The system according to embodiment 16, wherein the diameter of the primary coil is more than
15 cm.
18. The system according to embodiment 17, wherein the diameter of the primary coil is more than
20 cm.
19. The system according to embodiment 18, wherein the diameter of the primary coil is more than
30 cm.
20. The system according to embodiment 19, wherein the diameter of the primary coil is more than
50 cm.
21. The system according to any one of embodiments 1-15, wherein the area of the primary coil is more than 0.5 cm2.
22. The system according to embodiment 21, wherein the area of the primary coil is more than 2 cm2.
23. The system according to embodiment 22, wherein the area of the primary coil is more than 10 cm2.
24. The system according to embodiment 23, wherein the area of the primary coil is more than 100 cm2.
25. The system according to embodiment 24, wherein the area of the primary coil is more than 300 cm2.
26. The system according to embodiment 25, wherein the area of the primary coil is more than 500 cm2.
27. The system according to embodiment 26, wherein the area of the primary coil is more than 800 cm2.

### Aspect Large Coil B

1. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a drug delivery device for injecting a drug into a patient's body such as stimulation of penis erection
wherein
the drug is injected into the patient's body into at least one of both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising: an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising: an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising: a male sexual impotence treatment prosthesis apparatus, comprising
an operable prosthesis implantable in the cavities of the corpora cavernosa of an impotent patient to provide erect penile condition, when the prosthesis is operated, and an energy transmission device for wireless transmission of energy from outside the patient's body to inside the patient's body for use in connection with the operation of the prosthesis, when the prosthesis is implanted.
12. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising: an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a system for treating a female patient to avoid or promote pregnancy comprising a restriction device adapted to postoperatively restrict and release an oviduct of the patient.
18. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising: an obesity treatment device comprising:
at least one operable stretching device implantable in a patient and adapted to stretch a portion of the patient's stomach wall, and
an implantable operation device for operating the stretching device, when implanted, to stretch the stomach wall portion such that satiety is created,
wherein the operable stretching device is adapted to be placed against the outside of the stomach wall.
25. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a device for treating obesity of a patient, the device comprising:
at least one operable stretching device implantable in the patient and adapted to stretch a portion of the patient's stomach wall, and
an implantable control unit for controlling the operable stretching device, when the control unit and the stretching device are implanted, to stretch the stomach wall portion in connection with the patient eating such that satiety is created,
wherein the stretching device comprises a first engaging part and a second engaging part, wherein:
the first engaging part is adapted to be engaged to a first area of the stomach wall, and
the second engaging part is adapted to be engaged to a second area of the stomach wall, and
wherein
the stretching device is adapted to stretch a portion of the stomach wall between the first area and the second area.
26. A system for wirelessly charging an implantable medical device, when implanted in a body of a patient, the system comprising:
an internal energy receiver comprising a secondary coil, the internal energy receiver being connected to
the implantable medical device;
an external energy transmitter comprising a primary coil for wirelessly transmitting energy to the internal energy receiver via the secondary coil;
wherein a diameter of the primary coil is larger than a diameter of the secondary coil, the implantable medical device comprising:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
27. The system according to any one of the preceding embodiments, wherein the system further comprises:
an internal controller connected to the internal energy receiver, for controlling the amount of energy received by the internal energy receiver.
28. The system according to any one of the preceding embodiments, wherein the internal energy receiver further comprises a measurement unit for measuring a parameter related to the implantable medical device or the body of the patient.
29. The system according to any one of the preceding embodiments, wherein the controller is configured to measure the accumulated energy received by the internal energy receiver over a period of time and to measure a current change in energy received, and to control the energy received based on the accumulated energy and the current change.
30. The system according to any one of the preceding embodiments, wherein the controlled comprises a Proportional-Integral-Derivative, PID, regulator for controlling the received energy.
31. The system according to any one of the preceding embodiments, wherein the internal energy received comprises a variable impedance.
32. The system according to embodiment 6, when depending on any one of embodiments 28-31, wherein the internal energy receiver is configured to control the resonant frequency by controlling the variable impedance.
33. The system according to embodiment 32, wherein the controller is configured to vary the variable impedance in response to a measured parameter deviating from a predetermined interval or exceeding a threshold value.
34. The system according to embodiment 33, wherein the parameter relates to the energy received by the coil over a time period.
35. The system according to embodiment 32 or 33, wherein the measurement unit is configured to measure a parameter related to a change in energy received by the coil.
36. The system according to embodiment 28-35, wherein the receiving unit is configured to receive transferred energy in pulses according to a pulse pattern, and wherein the measurement unit is configured to measure a parameter related to the pulse pattern.
37. The system according to embodiment 28-36, wherein the receiving unit is configured to receive transferred energy in pulses according to a pulse pattern, and wherein the measurement unit is configured to measure a parameter related to the pulse pattern.
38. The system according to embodiment 28-37, wherein the controller is configured to control the variable impedance in response to the pulse pattern deviating from a predefined pulse pattern.
39. The system according to embodiment 28-38, wherein:
the variable impedance comprises a resistor and a capacitor,
the variable impedance comprises a resistor and an inductor,
the variable impedance comprises an inductor and a capacitor,
the variable impedance comprises a digitally tuned capacitor,
the variable impedance comprises a digital potentiometer, or
the variable impedance comprises a variable inductor.
40. The system according any one of the preceding embodiments, wherein the diameter of the primary coil is more than 0.5 cm.
41. The system according to embodiment 40, wherein the diameter of the primary coil is more than
10 cm.
42. The system according to embodiment 41, wherein the diameter of the primary coil is more than
15 cm.
43. The system according to embodiment 42, wherein the diameter of the primary coil is more than
20 cm.
44. The system according to embodiment 43, wherein the diameter of the primary coil is more than
30 cm.
45. The system according to embodiment 44, wherein the diameter of the primary coil is more than
50 cm.
46. The system according to any one of embodiments 28-39, wherein the area of the primary coil is more than 0.5 cm2.
47. The system according to embodiment 46, wherein the area of the primary coil is more than 2 cm2.
48. The system according to embodiment 47, wherein the area of the primary coil is more than 10 cm2.
49. The system according to embodiment 48, wherein the area of the primary coil is more than 100 cm2.
50. The system according to embodiment 49, wherein the area of the primary coil is more than 300 cm2.
51. The system according to embodiment 50, wherein the area of the primary coil is more than 500 cm2.
52. The system according to embodiment 51, wherein the area of the primary coil is more than 800 cm2.

### Aspect 456 NFMI Communication and Energy Transfer

1. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction.
2. The system according to embodiment 1, wherein:
   the internal communication unit comprises an internal NFMI receiver and an internal coil connected to the internal NFMI receiver, the internal NFMI receiver being configured to measure an induced voltage in the internal coil,
   the external communications unit comprises an external NFMI transmitter and an external coil connected to the external NFMI transmitter, and
   the external coil and the external NFMI transmitter are configured to modulate a magnetic field for sending data to the implantable medical device via the internal coil.
3. The system according to embodiment 2, wherein the external NFMI transmitter further comprises a capacitor for tuning the external coil and the external NFMI transmitter.
4. The system according to embodiment 3, wherein the internal NFMI receiver comprises a tunable resistor and capacitor tank for turning the internal coil and the internal NFMI receiver.
5. The system according to any one of the preceding embodiments, wherein:
the internal communication unit comprises an internal NFMI transmitter and an internal coil connected to the internal NFMI transmitter,
the external communications unit comprises an external NFMI receiver and an external coil connected to the external NFMI receiver, the external NFMI receiver being configured to measure an induced voltage in the external coil,
the internal coil and the internal NFMI transmitter are configured to modulate a magnetic field for sending data to the external communications unit via the external coil.
6. The system according to embodiment 5, wherein the internal NFMI transmitter further comprises a capacitor for tuning the internal coil and the internal NFMI receiver.
7. The system according to embodiment 5 or 6, wherein the external NFMI receiver comprises a tunable resistor and capacitor tank for turning the external NFMI receiver and the external coil.
8. The system according to any one of the preceding embodiments, wherein the implantable medical device comprises an active portion configured to monitor, treat or perform a function of a body of a patient.
9. The system according to embodiment 8, wherein the active portion is not a pacemaker, hearing aid or a neurostimulation implant.
10. The system according to any one of the preceding embodiments, wherein the internal communications unit is adapted to be implanted at a tissue depth of at least 8 cm or at least 15 cm.
11. The system according to any one of the preceding embodiments, wherein the internal communications unit is adapted to be implanted in an abdomen of a patient.
12. The system according to any one of the preceding embodiments, wherein the external communications unit is configured to communicate with another external device.
13. The system according to any one of the preceding embodiments, wherein the internal communications unit is configured to encrypt data before transmitting it to the external communications unit.
14. The system according to embodiment 13, wherein the external communications unit is configured to relay the encrypted data to the another external device without decrypting it.

### Aspect 456B NFMI

1. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a drug delivery device for injecting a drug into a patient's body such as stimulation of penis erection wherein
the drug is injected into the patient's body into at least one of
both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavernosa in close proximity thereto so as to supply the drugs through the catheter or the catheter may be adapted to be implanted in at least one of the corpora cavernosa so as to supply the drugs directly into the corpus cavernosum through the catheter.
6. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. A method for wireless energy transfer from an external energy source located outside the patient to an internal energy receiver located inside the patient, the internal energy receiver being connected to an implantable medical device for supplying received energy thereto, the method comprising:
determining an accumulated amount of received energy over a time period;
determining a current change in the received energy;
determining a control signal reflecting the accumulated received energy and the change in the transferred or received energy;
controlling the energy transfer based on the control signal,
wherein the implantable medical device comprises:
an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a male sexual impotence treatment prosthesis apparatus, comprising
an operable prosthesis implantable in the cavities of the corpora cavernosa of an impotent patient to provide erect penile condition, when the prosthesis is operated, and
an energy transmission device for wireless transmission of energy from outside the patient's body to inside the patient's body for use in connection with the operation of the prosthesis, when the prosthesis is implanted.
12. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a system for treating a female patient to avoid or promote pregnancy comprising a restriction device adapted to postoperatively restrict and release an oviduct of the patient.
18. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a device for treating obesity of a patient, the device comprising:
at least one operable stretching device implantable in the patient and adapted to stretch a portion of the patient's stomach wall, and
an implantable control unit for automatically controlling the operable stretching device, when the control unit and stretching device are implanted, to stretch the stomach wall portion in connection with the patient eating such that satiety is created,
wherein the stretching device comprises a first and a second engaging part, wherein:
the first part is adapted to be engaged to a first area of the stomach wall, and
the second part is adapted to be engaged to a second area of the stomach wall, and wherein
the stretching device is adapted to stretch a portion of the stomach wall between the first area and the second area.
24. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
an obesity treatment device comprising:
   at least one operable stretching device implantable in a patient and adapted to stretch a portion of the patient's stomach wall, and
   an implantable operation device for operating the stretching device, when implanted, to stretch the stomach wall portion such that satiety is created,
   wherein the operable stretching device is adapted to be placed against the outside of the stomach wall.
25. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
the second engaging part is adapted to be engaged to a second area of the stomach wall, and wherein
the stretching device is adapted to stretch a portion of the stomach wall between the first area and the second area.
26. A system for communication with an implantable medical device, when implanted in a body of a patient, comprising:
an internal communications unit, connected to or comprised in the implantable medical device;
an external communications unit,
wherein the internal communications unit and the external communications unit are configured to communicate using near field magnetic induction,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
26. The system according to any one of the preceding embodiment, wherein:
   the internal communication unit comprises an internal NFMI receiver and an internal coil connected to the internal NFMI receiver, the internal NFMI receiver being configured to measure an induced voltage in the internal coil,
   the external communications unit comprises an external NFMI transmitter and an external coil connected to the external NFMI transmitter, and
   the external coil and the external NFMI transmitter are configured to modulate a magnetic field for sending data to the implantable medical device via the internal coil.
28. The system according to embodiment 27, wherein the external NFMI transmitter further comprises a capacitor for tuning the external coil and the external NFMI transmitter.
29. The system according to embodiment 28, wherein the internal NFMI receiver comprises a tunable resistor and capacitor tank for turning the internal coil and the internal NFMI receiver.
30. The system according to any one of the preceding embodiments, wherein:
   the internal communication unit comprises an internal NFMI transmitter and an internal coil connected to the internal NFMI transmitter,
   the external communications unit comprises an external NFMI receiver and an external coil connected to the external NFMI receiver, the external NFMI receiver being configured to measure an induced voltage in the external coil,
   the internal coil and the internal NFMI transmitter are configured to modulate a magnetic field for sending data to the external communications unit via the external coil.
31. The system according to embodiment 30, wherein the internal NFMI transmitter further comprises a capacitor for tuning the internal coil and the internal NFMI receiver.
32. The system according to embodiment 30 or 31, wherein the external NFMI receiver comprises a tunable resistor and capacitor tank for turning the external NFMI receiver and the external coil.
33. The system according to any one of the preceding embodiments, wherein the implantable medical device comprises an active portion configured to monitor, treat or perform a function of a body of a patient.
34. The system according to embodiment 33, wherein the active portion is not a pacemaker, hearing aid or a neurostimulation implant.
35. The system according to any one of the preceding embodiments, wherein the internal communications unit is adapted to be implanted at a tissue depth of at least 8 cm or at least 15 cm.
36. The system according to any one of the preceding embodiments, wherein the internal communications unit is adapted to be implanted in an abdomen of a patient.
37. The system according to any one of the preceding embodiments, wherein the external communications unit is configured to communicate with another external device.
38. The system according to any one of the preceding embodiments, wherein the internal communications unit is configured to encrypt data before transmitting it to the external communications unit.
38. The system according to embodiment 38, wherein the external communications unit is configured to relay the encrypted data to the another external device without decrypting it.

### Aspect 459 Resonant Circuit

1. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit.
2. The implantable medical device according to embodiment 1, wherein the first energy receiving unit has a first resonant frequency based on the inductance of the first coil and the impedance of the first impedance unit, and the second energy receiving unit has a second resonant frequency based on the inductance of the second coil and the impedance of second impedance unit.
3. The implantable medical device according to embodiment 2, wherein the first receiving unit has a resonant frequency different from the resonant frequency of the second receiving unit.
4. The implantable medical device according to any preceding embodiment, wherein the first and second impedance units are connected in parallel to the respective coil.
5. An implantable medical device adapted to receive transcutaneously and wirelessly transmitted energy, the implantable medical device comprising:
   an energy consuming part,
a receiving unit configured for receiving transcutaneously transferred energy and transferring the received energy to the energy consuming part, comprising:
   a first coil portion and a second coil portion, and
   a first impedance unit and a second impedance unit, wherein the first impedance unit is connected to the first coil portion and the second impedance unit is connected to the second coil portion,
a measurement unit configured to measure a parameter related to energy transfer, and
a controller configured to control the subcutaneously received energy based on the parameter by controlling the first or the second impedance unit.
6. The implantable medical device according to embodiment 5, wherein the first coil portion and the second coil portion are at least one of portions of the same coil, or portions or different coils connected in series.
7. The implantable medical device according to embodiment 5 or 6, wherein the first coil portion and the second coil portion have the same inductance, or the first coil portion has a different inductance than the second coil portion.
8. The implantable medical device according to any one of embodiments 5-7, wherein the first impedance is connected in parallel to the first coil portion and the second impedance is connected in parallel to the second coil portion.
9. The implantable medical device according to any one of embodiments 5-8, wherein one of the first coil portion and the second coil portion are overlapping the other of the first coil portion and the second coil portion, or the first coil portion and the second coil portion are not overlapping with the other of the first coil portion and the second coil portion.
10. The implantable medical device according to any one of embodiments 5-9, wherein the first coil portion and the first impedance unit has a first resonance frequency, and the second coil portion and the second impedance unit has a second resonance frequency.
11. The implantable medical device according to embodiment 10, wherein the first resonance frequency is different from the second resonance frequency.
12. The implantable medical device according to any preceding embodiment, wherein the first or second impedance unit is a capacitor.
13. The implantable medical device according to any preceding embodiment, wherein the first impedance unit and the second impedance unit have different impedances.
14. An implantable medical device comprising:
   an energy consuming part,
a first receiving unit comprising:
   a first coil configured for receiving transcutaneously transferred energy and transferring the received energy to the energy consuming part,
   a first impedance electrically connected to the coil,
a second receiving unit comprising:
   a second coil portion and a third coil portion configured for receiving transcutaneously transferred energy and transferring the received energy to the energy consuming part, and
   a second impedance unit and a third impedance unit, wherein the second impedance unit is connected to the second coil portion and the third impedance unit is connected to the third coil portion,
a measurement unit configured to measure a parameter related to energy transfer, and
a controller configured to control the subcutaneously received energy based on the parameter by controlling the first, the second or the third impedance unit.

### Aspect 459B Resonant Circuit

1. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
   wherein the implantable medical device comprises:
an implantable energized medical device configured to be held in position by a tissue portion of a patient, the medical device comprising:
   a first portion configured to be placed on a first side of the tissue portion, the first portion having a first cross-sectional area in a first plane and comprising a first surface configured to face a first tissue surface of the first side of the tissue portion,
   a second portion configured to be placed on a second side of the tissue portion, the second side opposing the first side, the second portion having a second cross-sectional area in a second plane and comprising a second surface configured to engage a second tissue surface of the second side of the tissue portion, and
   a connecting portion configured to be placed through a hole in the tissue portion extending between the first and second sides of the tissue portion, the connecting portion having a third cross-sectional area in a third plane and a fourth cross-sectional area in a fourth plane and a third surface configured to engage the first tissue surface of the first side of the tissue portion,
wherein the connecting portion is configured to connect the first portion to the second portion, wherein:
   the first, second, third and fourth planes are parallel to each other,
   the third cross-sectional area is smaller than the first, second and fourth cross-sectional areas, such that the first portion, second portion and connecting portion are prevented from travelling through the hole in the tissue portion in a direction perpendicular to the first, second and third planes, and
   the first portion is detachably connected to at least one of the connecting portion and the second portion.
2. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
   wherein the implantable medical device comprises:
a system for treating a patient having a disorder related to the patient's intestine, the system comprises
an artificial intestine section adapted to being implanted inside a patient's body, along with an accumulator for accumulating energy, the artificial intestine section has a first open end portion and a second open end portion in flow communication with one another, wherein at least the first open end portion and possibly also the second open end portion is adapted to being connected to a surgically created opening in the patient's intestine, and
the accumulator is adapted to be charged wirelessly with energy and to be arranged so as to supply energy directly or indirectly to at least one energy consuming part of said artificial intestine section.
3. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by
controlling the first or the second impedance unit,
wherein the implantable medical device comprises:
a system that acts on an intestinal reservoir formed from surgically modified intestine that has been cut along a mutual contact line of laterally adjacent sections of a bent portion of intestine and connected so that the resulting upper and lower halves of the intestine form an intestinal wall of the reservoir, the system comprises
an artificial flow control device implantable in the patient's body and adapted to control flow of the intestinal contents from said reservoir
the artificial flow control device comprises at least one pump adapted to act on said intestinal wall so as to reduce the reservoir's volume in order to empty the reservoir.
4. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an infusion device comprising an infusion needle and a drive unit coupled to the infusion needle and arranged for advancing the tip end of the infusion needle to penetrate fibrosis when the device is implanted in the patient's body, wherein at least the infusion needle and the drive unit are sized and formed for implantation in the patient's body.
5. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a drug delivery device for injecting a drug into a patient's body such as stimulation of penis erection wherein
the drug is injected into the patient's body into at least one of
both a right and left corpus cavernosum,
two deep arteries of the right and left corpus cavernosum,
muscle tissue regulating blood flow through the right and left corpus cavernosum, and
tissue in close proximity to the left and right corpus cavernosum,
wherein the drug delivery device comprises a catheter adapted to be implanted outside the corpora cavemosa in close proximity thereto so as to supply the drugs through the catheter or the catheter may be adapted to be implanted in at least one of the corpora cavemosa so as to supply the drugs directly into the corpus cavernosum through the catheter.
6. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable lubrication device comprising:
a reservoir that stores a lubricating fluid and,
a fluid connection that introduces the stored lubricating fluid into a damaged joint when the lubrication device is implanted in a patient's body,
wherein the lubricating device is configured to be completely implanted into the patient's body such that a damaged joint can post-operatively be lubricated from within the patient's body,
and wherein the operative supply of lubricating fluid to the damaged joint is controlled continuously, intermittently, periodically or depending on a physical parameter of the patient, such as a fluid level within the joint.
7. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a device for bone adjustment in a mammal, comprising
two or more anchoring devices for attaching to a bone in said mammal,
an adjustment device for exerting force on said anchoring devices to adjust the distance between or orientation of at least two of said anchoring devices,
wherein
said anchoring devices and said adjustment device are implanted intramedullary in said mammal and wherein said adjustment device is constructed to postoperatively adjust said distance, and
the adjustment is a lengthening of a bone, a healing of a fracture, a changing of a bone angle, a reshaping of a bone, a compression of a bone, a torsion of a bone, or a combination thereof.
8. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable drain adapted to move body fluid or a hydraulic reservoir with hydraulic treatment fluid to move hydraulic fluid, from one part of the body to another part of the body, a fluid movement device that which is completely implanted and which does not have any mechanical structure penetrating through the skin of the patient is obtained.
9. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an apparatus for drainage of a body fluid or movement of hydraulic treatment fluid in a human or mammal patient comprising
a fluid movement device for pumping hydraulic treatment fluid or body fluid, the fluid movement device being powered by an energy source and powered by an electrical or a hydraulic motor
at least one connecting tube connected to the fluid movement device so that the fluid movement device and the tube form a drainage or hydraulic arrangement
wherein the hydraulic arrangement is adapted to be implanted inside the body of the patient, and placed so that the tube interconnects one part of the body with another part of the body and where fluid movement device is adapted to suck body fluid from the one part of the body via the tube to the other part of the body.
10. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable device for improving the pump function of the heart of a human patient by applying an external force on the heart muscle, said device comprising
at least one heart contacting organ, periodically exerting force onto the heart following the heart contractions and adding force thereto,
a drive unit to create kinetic movement to be used by the heart contacting organ,
a fixation device adapted to be mounted in a stable position to human bone allowing said drive unit and kinetic movement to get necessary contra force,
wherein said drive unit further comprises a respiration movement compensator for compensating for the respiratory movement of the heart in relation to the stable bone position, and said drive unit is adapted to allow a movement to compensate for the respiratory movement in relation between said heart contacting organ and said bone.
11. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a male sexual impotence treatment prosthesis apparatus, comprising
an operable prosthesis implantable in the cavities of the corpora cavernosa of an impotent patient to provide erect penile condition, when the prosthesis is operated, and
an energy transmission device for wireless transmission of energy from outside the patient's body to inside the patient's body for use in connection with the operation of the prosthesis, when the prosthesis is implanted.
12. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a device for treatment or monitoring of an aneurysm, comprising
an implantable member adapted to hold fluid, the member being adapted to be placed in connection with a blood vessel having the aneurysm and to exercise a pressure on the aneurysm,
a control unit adapted to control pressure adjustments of the implantable member.
13. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable blood clot removal device for removing blood clots from the vascular system of a patient comprising:
a blood flow passageway to be connected to the patient's vascular system to allow circulation of the patient's blood through the blood flow passageway,
a filter provided in the blood flow passageway for collecting blood clots occurring in the blood flowing through the blood flow passageway, and
a cleaning device for moving blood clots collected by the filter out of the blood flow passageway.
14. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable apparatus for treating urinary retention of a mammal patient comprising:
an implantable powered member adapted exert a force from the outside on a selected part of the urinary bladder in order to discharge urine from the urinary bladder, and
a control device for controlling the operation of the powered member.
15. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a breast implant system comprises a plurality of chambers including one or more first fluid chambers and one or more second fluid chambers, wherein:
at least the first fluid chamber is to be implanted in the human body to form part of a breast implant,
the second fluid chambers is implanted to form part of the breast implant or implanted inside the patient's body remote from the breast implant,
the first fluid chamber is interconnected with the second fluid chamber, such that fluid can be exchanged between the first and second fluid chambers, so as to change their respective fluid content.
16. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an apparatus for treating obesity and/or reflux comprising at least one volume filling device adapted to be at least substantially invaginated by a stomach wall portion of the patient, wherein the volume filling device is adapted to be placed outside of the stomach wall with the outer surface of the volume filling device resting against the outside of the stomach wall, such that the volume of the food cavity is reduced in size by a volume substantially exceeding the volume of the volume filling device.
17. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a system for treating a female patient to avoid or promote pregnancy comprising a restriction device adapted to postoperatively restrict and release an oviduct of the patient.
18. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an apparatus for controlling a flow of sperms in an uterine tube of a female patient, the apparatus comprising: an implantable constriction device for constricting at least one portion of the uterine tube wall to at least partly constrict the uterine tube to influence the flow of sperms in the uterine tube.
19. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an implantable medical device for lubrication of a synovial joint having a joint cavity, the implantable device comprising a solid lubricant and a feeding device, wherein said feeding device is adapted to feed said solid lubricant into the joint cavity for lubricating the synovial joint.
20. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an artificial valve for implantation in a mammal body, in or adjacent to a mammal blood vessel, the artificial valve comprises a casing and an opening and closing mechanism, at least part of the opening and closing mechanism comprises a first moving part adapted to make an opening and a closing movement relative to the casing, the movements comprising movements to assume an open and a closed position for opening and closing, respectively, the blood flow through said blood vessel.
21. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an apparatus adapted to control the flow of fluids and/or other bodily matter in a lumen that is formed by the tissue wall of a bodily organ, the apparatus comprising an implantable constriction device for constricting a portion of the tissue wall to influence the flow in the lumen, a stimulation device for stimulating the wall portion of the tissue wall, and a control device for controlling the stimulation device to stimulate the wall portion as the constriction device constricts the wall portion to cause contraction of the wall portion to further influence the flow in the lumen.
22. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
an apparatus for treating obesity of an patient having a stomach with a food cavity, the apparatus comprising:
   a volume filling device adapted to be at least substantially invaginated by a stomach wall portion of the patient with the outer surface of the volume filling device resting against the stomach wall, such that the volume of the food cavity is reduced in size by a volume substantially exceeding the volume of the volume filling device,
   at least one adjustable stretching device adapted to be at least substantially invaginated by a stomach wall portion of the patient with the outer surface of the stretching device resting against the stomach wall and adapted to stretch a portion of stomach wall, and
   a fluid connection device interconnecting the volume filling device and the stretching device.
23. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a device for treating obesity of a patient, the device comprising:
   at least one operable stretching device implantable in the patient and adapted to stretch a portion of the patient's stomach wall, and
   an implantable control unit for automatically controlling the operable stretching device, when the control unit and stretching device are implanted, to stretch the stomach wall portion in connection with the patient eating such that satiety is created,
   wherein the stretching device comprises a first and a second engaging part, wherein:
   the first part is adapted to be engaged to a first area of the stomach wall, and
   the second part is adapted to be engaged to a second area of the stomach wall, and wherein
   the stretching device is adapted to stretch a portion of the stomach wall between the first area and the second area.
24. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises: an obesity treatment device comprising:
   at least one operable stretching device implantable in a patient and adapted to stretch a portion of the patient's stomach wall, and
   an implantable operation device for operating the stretching device, when implanted, to stretch the stomach wall portion such that satiety is created,
   wherein the operable stretching device is adapted to be placed against the outside of the stomach wall.
25. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a device for treating obesity of a patient, the device comprising:
   at least one operable stretching device implantable in the patient and adapted to stretch a portion of the patient's stomach wall, and
   an implantable control unit for controlling the operable stretching device, when the control unit and the stretching device are implanted, to stretch the stomach wall portion in connection with the patient eating such that satiety is created,
   wherein the stretching device comprises a first engaging part and a second engaging part, wherein:
   the first engaging part is adapted to be engaged to a first area of the stomach wall, and
   the second engaging part is adapted to be engaged to a second area of the stomach wall, and wherein
   the stretching device is adapted to stretch a portion of the stomach wall between the first area and the second area.
26. An implantable medical device adapted to receive transcutaneously and wirelessly transmitted energy, the implantable medical device comprising:
an energy consuming part,
a first energy receiving unit, comprising a first coil configured for receiving transcutaneously transferred energy, and a first impedance unit electrically connected to the first coil, the receiving unit being configured to transfer the received energy to the energy consuming part
a second energy receiving unit, comprising a second coil configured for receiving transcutaneously transferred energy and a second impedance unit electrically connected to the second coil, the receiving unit being configured to transfer the received energy to the energy consuming part,
a measurement unit configured to measure a parameter related to energy transfer, and
a controller configured to control the subcutaneously received energy based on the parameter by controlling the first or the second impedance unit,
wherein the implantable medical device comprises:
a device for treating obesity of a patient, the device comprising:
a. at least one operable stretching device implantable in a patient operating via mechanical, pneumatic, and/or hydraulic action to stretch a portion of the patient's stomach wall, and
b. an implantable control unit for automatically controlling the operable stretching device, when the control unit and stretching device are implanted, to stretch the stomach wall portion in connection with the patient eating such that satiety is created.
27. The implantable medical device according to any one of the preceding embodiments, wherein the first energy receiving unit has a first resonant frequency based on the inductance of the first coil and the impedance of the first impedance unit, and the second energy receiving unit has a second resonant frequency based on the inductance of the second coil and the impedance of second impedance unit.
28. The implantable medical device according to embodiment 27, wherein the first receiving unit has a resonant frequency different from the resonant frequency of the second receiving unit.
29. The implantable medical device according to any preceding embodiment, wherein the first and second impedance units are connected in parallel to the respective coil. Aspect 453C Voice control
2. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
3. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
wherein the implantable medical device comprises:
a breast implant system, comprising:
a plurality of chambers including
at least one first fluid chamber (1) which is adapted to allow an increase and a decrease of its volume, and
at least one second fluid chamber (2) which is adapted to allow an increase and a decrease of its volume,
said first and second fluid chambers (1, 2) being adapted for implantation in the human body with the at least one first fluid chamber (1) forming part of a breast implant (10),
said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
4. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
wherein the implantable medical device comprises:
a breast implant system comprising:
at least one casing with a flexible outer shape for permanent implantation in a patient's body so as to form part of a breast implant,
at least one first element contained in the casing, and
a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
5. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
wherein the implantable medical device comprises:
a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
6. A method of teaching a voice-controlled medical implant to recognize a voice command, the method comprising:
inputting a first audio training phrase to the medical implant, when the medical implant is implanted in the body of the patient,
creating a transfer function, the transfer function being based on the first audio training phrase, wherein the transfer function is configured to adjust the amplitude of at least one frequency of audio received at the medical device for enhancing audio received at the medical implant to facilitate detection of voice commands,
inputting a second audio training phrase to the medical implant, the second audio training phrase comprising the voice command, the voice command comprising an instruction for the control of the medical implant,
using the transfer function for generating an enhanced second audio training phrase in the medical implant, and
associating the enhanced second audio training phrase with the instruction for the control of the medical implant,
wherein the implantable medical device comprises:
an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
7. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
8. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
9. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
10. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
11. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
12. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
13. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
14. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
15. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
16. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
17. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
18. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
19. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
20. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
21. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
22. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
23. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
24. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
25. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
26. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
27. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
28. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
29. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
30. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
31. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
32. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body, said infusion needle and drive unit being designed for implantation in a patient's body, wherein the tip end of the infusion needle is laterally movable within the body of the device for variation of an injection site.
33. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
34. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
35. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
36. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
37. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
38. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
39. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
40. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
41. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
42. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the apparatus is wherein the force of the powered member exerted on the urinary bladder is at least partly exerted against the support structure when discharging urine from the urinary bladder.
43. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
44. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
45. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
46. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
47. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
48. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
49. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
50. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
51. A method of teaching a voice-controlled medical implant to recognize a voice command, the method comprising:
   inputting a first audio training phrase to the medical implant, when the medical implant is implanted in the body of the patient,
   creating a transfer function, the transfer function being based on the first audio training phrase, wherein the transfer function is configured to adjust the amplitude of at least one frequency of audio received at the medical device for enhancing audio received at the medical implant to facilitate detection of voice commands,
   inputting a second audio training phrase to the medical implant, the second audio training phrase comprising the voice command, the voice command comprising an instruction for the control of the medical implant,
   using the transfer function for generating an enhanced second audio training phrase in the medical implant, and
   associating the enhanced second audio training phrase with the instruction for the control of the medical implant,
   wherein the implantable medical device comprises:
   a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
51. The method according to any one of the preceding embodiments, wherein adjusting the amplitude comprises at least one of: filtering, cancelling and amplifying the at least one frequency.
52. The method according to any one of the preceding embodiments, wherein at least one of the first and second audio training phrase is a spoken audio training phrase.
53. The method according to embodiment 53, wherein the spoken audio training phrase is spoken by the patient the implant is implanted in.
54. The method according to any one of the preceding embodiments, wherein the first audio training phrase comprises the at least one voice command related to an instruction for the control of the medical implant.
55. The method according to any one of the preceding embodiments, wherein the first and second audio training phrases is the same voice command.
56. The method according to any one of the preceding embodiments, wherein the first and second audio training phrases are different.
57. The method according to any one of the preceding embodiments, wherein creating the transfer function comprises amplifying frequencies muffled by the location of the medical implant in the body of the patient.
58. The method according to any one of the preceding embodiments, wherein creating the transfer function comprises filtering or cancelling noise generated by the body.
59. The method according to any one of the preceding embodiments, wherein the medical implant is configured to receive voice commands related to an instruction for control of the medical implant.
60. The method according to any one of the preceding embodiments, wherein the voice command relates to at least one of:
   performing a function of the medical device;
   using a sensor to measure a parameter relating to a condition of the patient or a condition of the medial implant;
   sending or receiving data from the medical implant.

### Aspect 454C Dual Remote Controls

1. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for bone adjustment in a mammal, the device comprising:
a bone adjusting housing, sized and adapted to be implanted entirely intramedullary in a femoral or tibial bone in said mammal said housing comprising
two or more anchoring devices adapted to be attached to the femoral or tibial bone in said mammal,
an adjustment device for exerting a force on said two or more anchoring devices, said adjustment device being adapted to postoperatively adjust at least one of a distance between or orientation of, at least two of said two or more anchoring devices,
an operation device comprising a motor, wherein said operation device is adapted to operate said adjustment device,
a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
a control device adapted to control the operation device, wherein the control device is programmed to control the device according to a pre-programmed time-schedule comprising a program of incremental changes.
2. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a breast implant system, comprising:
a plurality of chambers including
at least one first fluid chamber (1) which is adapted to allow an increase and a decrease of its volume, and
at least one second fluid chamber (2) which is adapted to allow an increase and a decrease of its volume,
said first and second fluid chambers (1, 2) being adapted for implantation in the human body with the at least one first fluid chamber (1) forming part of a breast implant (10),
said fluid chambers (1, 2) being interconnected when implanted such that fluid can be exchanged between the first and second fluid chambers (1, 2) so as to change their respective fluid content, and
a pump (5) adapted for pumping fluid between the first and second fluid chambers (1, 2).
3. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a breast implant system comprising:
at least one casing with a flexible outer shape for permanent implantation in a patient's body so as to form part of a breast implant,
at least one first element contained in the casing, and
a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a pregnancy control system for promoting pregnancy of a female human patient, comprising:
an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
the restriction device (2) is adapted to be positioned on the outside of the oviduct (31a, 31b); and
the pregnancy control system further comprises a control device (32, 33) operable from outside the patient's body to control the restriction device, wherein the control device comprises an internal control unit (33) adapted to be implanted in the patient's body, the internal control unit controlling the restriction device,
wherein the restriction device comprises at least two restriction areas adapted to restrict the oviduct lumen at different places on the oviduct, the restriction device being adapted to change the restriction of the oviduct lumen over time from one restriction area to another restriction area.
19. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
an electrical stimulation device (3) configured to electrically stimulate at least a first and a second portion of a wall of the uterine tube for at least restricting the uterine tube, and
a flow control device (4) configured to control the electrical stimulation device (3) to electrically stimulate the first and second portion of the wall of the uterine tube in succession for influencing the flow of sperms in the uterine tube to control pregnancy.
20. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a filtering device for removing particles from a bodily fluid of a patient, the filtering device being implantable in the patient's body and comprising:
a tube forming a main fluid passageway for bodily fluid, through which the bodily fluid of the patient passes when the tube is implanted in the patient,
a filter connected to the tube and, when in use, arranged for filtering the bodily fluid passing through said main fluid passageway and collecting said particles to be removed passing through the tube, and
a filter cleaning device for cleaning the filter, the filter cleaning device being in contact with the filter and configured to remove particles from the filter via a movement of the filter cleaning device, and
an implantable chamber arranged and adapted for receiving the removed particles from said filter cleaning device via the movement of the filter cleaning device,
wherein the filter comprises a plurality of openings configured to allow the bodily fluid to pass through the filter.
22. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a filtering device for removing particles from a bodily fluid of a patient, the filtering device being implantable in the patient's body and comprising:
a tube forming a main fluid passageway for bodily fluid, through which the bodily fluid of the patient passes when the tube is implanted in the patient,
a filter connected to the tube and, when in use, arranged for filtering the bodily fluid passing through said main fluid passageway and collecting said particles to be removed passing through the tube,
wherein the filter comprises a plurality of openings configured to allow the bodily fluid to pass through the filter, and
a filter cleaning device for cleaning the filter, the filter cleaning device being in contact with the filter and configured to remove particles from the filter via a movement of the filter cleaning device.
23. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a filtering device for removing particles from a fluid of a patient, the filtering device being implantable in the patient's body and comprising:
a tube forming a fluid passageway, and
an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a filtering device for removing particles from a fluid of a patient, the filtering device being implantable in the patient's body and comprising:
a tube forming a fluid passageway, and
an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus for moving a fluid to or from a cavity in a body of a human or mammal patient, comprising:
a fluid movement device;
an energy source adapted to supply energy to said fluid movement device;
at least one connecting tube having a tube end, the at least one connecting tube being connected to the fluid movement device; and
a flexible patch;
wherein:
the fluid movement device and the at least one connecting tube form a fluid moving arrangement adapted to be implanted inside the body of the human or mammal patient;
the tube end is adapted to be inserted into an organ of the patient;
at least a portion of the apt least one connecting tube is adapted to be invaginated in a wall of the organ so as to interconnect a treatment area of the body with a delivery area;
the fluid movement device is adapted to move fluid from the treatment area via the at least one connecting tube to the delivery area;
the flexible patch comprises a net structure adapted to be overgrown by human fibrotic tissue when implanted in the human or mammal patient; and
the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body, said infusion needle and drive unit being designed for implantation in a patient's body,
wherein the tip end of the infusion needle is laterally movable within the body of the device for variation of an injection site.
32. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises: an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
wherein the infusion device is completely implantable in a patient's body.
33. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an infusion device implantable in a patient's body, the infusion device comprising:
a body implantable in the patient's body and having an outer wall,
an infusion needle disposed within the body of the infusion device and having a tip end, and
a drive unit coupled to the infusion needle which advances and retracts the tip end of the infusion needle to penetrate any fibrosis surrounding the infusion device, when the infusion device is implanted in the patient's body,
said infusion needle and drive unit being implantable in the patient's body, the drive unit being operable when implanted in the patient's body,
wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an implantable lubrication device for lubricating a joint of a human or mammal patient by adding and circulating lubricating fluid, the lubrication device comprises:
a reservoir adapted to store an added lubricating fluid, and
a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus for treating obesity of an obese patient having a stomach with a food cavity, the apparatus comprising:
at least one inflatable volume filling device adapted to be completely invaginated by a stomach wall portion of the patient,
wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment prosthesis apparatus, comprising:
an operable prosthesis implantable in the cavities of the corpora cavernosa of an impotent patient to provide erect penile condition, when the prosthesis is operated,
an energy transmission device for wireless transmission of energy of a first form from outside the patient's body to inside the patient's body for use in connection with the operation of the prosthesis, when the prosthesis is implanted, and
an energy transforming device implantable in the patient for transforming the energy of the first form wirelessly transmitted by the energy transmission device into energy of a second form, the penile prosthesis being operable in response to the energy of the second form,
wherein the energy transforming device includes at least one element having a positive region and a negative region, the element creates an energy field between the positive and negative regions when exposed to the energy of the first form transmitted by the energy transmission device, and the energy field produces the energy of the second form.
40. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a male sexual impotence treatment prosthesis—an operable non-inflatable penile prosthesis implantable in cavities of corpora cavernosa of an impotent patient's body to provide an erect penile condition, when the prosthesis is implanted,
a source of energy implantable in the patient's body for supplying energy for operating the penile prosthesis,
a control device operable from outside the patient's body for controlling a supply of energy from the source of energy, to control an erect and a flaccid state of the patient's penis, and
an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
an apparatus adapted to treatment of urinary retention of a mammal patient by discharging urine from the urinary bladder, comprising:
an implantable powered member (100) with a urinary bladder surface contact part, said powered member is adapted to exert a force from the outside on a selected part of the urinary bladder in order to discharge urine from the urinary bladder;
a support structure adapted to support against at least one of, a bone or other human tissue, such as the pelvic bone, pubic bone or sacrum, other human tissue such as peritoneum, the abdominal or pelvic wall or the urine bladder itself; and
a control device (200) for controlling the operation of the powered member,
wherein the apparatus is wherein the force of the powered member exerted on the urinary bladder is at least partly exerted against the support structure when discharging urine from the urinary bladder.
42. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
   an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for treating an aneurysm of a human or mammal patient comprising:
an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
a pressure regulator adapted to regulate the pressure in the member
a sensor or measuring device for sensing or measuring an expansion of the aneurysm
a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user, wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user, wherein the implantable medical device comprises:
a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A communication system for transmission of data to or from an implantable medical device, the communication system comprising:
   an implantable medical device;
   a first remote control comprising a first wireless communication unit configured for wireless transmission of data to or from the implantable medical device, the first remote control being operable by a user; and
   a second remote control comprising a second wireless communication unit configured for wireless transmission of control commands or data to or from the implantable medical device, and a third communication unit for communicating with a patient display device, the second remote control being inoperable by a user,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
move the removed blood clots away from the blood flow passageway.
51. The communication system according to any one of the preceding embodiments, wherein the first remote control comprises an input device for receiving a first user input, and wherein the first remote control is configured to transmit the first user input to the implantable medical device.
52. The communication system according to any one of the preceding embodiments, wherein the second remote control is configured to receive second user input from the patient display device and to transmit the second user input to the implantable medical device.
53. The communication system according to any one of the preceding embodiments, wherein the data comprises a control command for the medical device.
54. The communication system according to any one of the preceding embodiments, wherein at least one of the first wireless communication unit and the second wireless communication unit is configured to send or receive data using near-field magnetic induction.
55. The communication system according to embodiment 54, wherein at least one of the first wireless communication unit and the second wireless communication unit comprises a transmitter coil for modulating a magnetic field for transmitting the data, and wherein the implantable medical device comprises a receiving coil and an NFMI receiver connected to the receiving coil to receive the data.
56. The communication system according to embodiment 55, wherein the transmitter coil is configured to modulate a magnetic field, and the NFMI receiver is adapted to measure the magnetic field in the receiving coil.
57. The communication system according to any one of the preceding embodiments, wherein at least one of the first wireless communication unit and the second wireless communication unit is configured to wirelessly charge the medical device using near-field magnetic induction.
58. The communication system according to embodiment 57, wherein the medical device comprises a coil for receiving wireless energy for charging the implant via near-field magnetic induction.
59. The communication system according to any one of the preceding embodiments, wherein the second and third communication units are configured to transmit and/or receive data using different network protocols.
60. The communication system according to any one of the preceding embodiments, wherein the second and third communication units are configured to transmit and/or receive data using different frequency bands.
61. The communication system according to any one of the preceding embodiments, wherein at least one of the first remote control, the second remote control and the implantable medical device comprises a Bluetooth transceiver.
62. The communication system according to any one of the preceding embodiments, wherein at least one of first remote control, the second remote control and the implantable medical device comprises a UWB transceiver.
63. The communication system according to embodiment 62, wherein the network protocol is one from the list of Radio Frequency type protocol, RFID type protocol, WLAN type protocol, Bluetooth type protocol, BLE type protocol, NFC type protocol, 3G/4G/5G type protocol, and GSM type protocol.
64. The communication system according to any one of the preceding embodiments, wherein the second communication unit has a longer effective range than the third communication unit.
65. The communication system according to any one of the preceding embodiments, wherein the second remote control is configured to communicate with a consumer electronics device.
66. The communication system according to embodiment 16, wherein the patient display device comprises the consumer electronics device.
67. The communication system according to any one of the preceding embodiments, wherein the first remote control is configured to control functions of the implantable medical device based on user input to the first remote control.

### Aspect 456C NFMI Communication and Energy Transfer

6. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
7. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
8. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
9. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
10. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
11. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
12. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
13. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
14. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
15. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
16. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
17. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
18. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
19. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
20. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
21. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
22. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
23. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
24. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
25. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
26. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
27. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
28. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
29. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
30. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
31. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an apparatus for moving a fluid to or from a cavity in a body of a human or mammal patient, comprising:
   a fluid movement device;
   an energy source adapted to supply energy to said fluid movement device; at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
32. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
33. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
34. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
35. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
36. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body, said infusion needle and drive unit being designed for implantation in a patient's body,
   wherein the tip end of the infusion needle is laterally movable within the body of the device for variation of an injection site.
37. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
38. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an infusion device implantable in a patient's body, the infusion device comprising:
   a body implantable in the patient's body and having an outer wall, an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
39. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
40. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
41. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
42. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
43. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
44. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
45. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
46. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member, wherein the force of the powered member exerted on the urinary bladder is at least partly exerted against the support structure when discharging urine from the urinary bladder.
47. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
48. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
      wherein the implantable medical device comprises:
   an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
49. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
50. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
51. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
52. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
53. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
54. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
55. A system for communication with an implantable medical device, when implanted in a body of a patient, comprising:
   an internal communications unit, connected to or comprised in the implantable medical device;
   an external communications unit,
   wherein the internal communications unit and the external communications unit are configured to communicate using near field magnetic induction,
   wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
56. The system according to any one of the preceding embodiments, wherein:
   the internal communication unit comprises an internal NFMI receiver and an internal coil connected to the internal NFMI receiver, the internal NFMI receiver being configured to measure an induced voltage in the internal coil,
   the external communications unit comprises an external NFMI transmitter and an external coil connected to the external NFMI transmitter, and the external coil and the external NFMI transmitter are configured to modulate a magnetic field for sending data to the implantable medical device via the internal coil.
57. The system according to embodiment 56, wherein the external NFMI transmitter further comprises a capacitor for tuning the external coil and the external NFMI transmitter.
58. The system according to embodiment 57, wherein the internal NFMI receiver comprises a tunable resistor and capacitor tank for turning the internal coil and the internal NFMI receiver.
59. The system according to any one of the preceding embodiments, wherein: the internal communication unit comprises an internal NFMI transmitter and an internal coil connected to the internal NFMI transmitter,
the external communications unit comprises an external NFMI receiver and an external coil connected to the external NFMI receiver, the external NFMI receiver being configured to measure an induced voltage in the external coil,
the internal coil and the internal NFMI transmitter are configured to modulate a magnetic field for sending data to the external communications unit via the external coil.
60. The system according to embodiment 59, wherein the internal NFMI transmitter further comprises a capacitor for tuning the internal coil and the internal NFMI receiver.
61. The system according to embodiment 59 or 60, wherein the external NFMI receiver comprises a tunable resistor and capacitor tank for turning the external NFMI receiver and the external coil.
62. The system according to any one of the preceding embodiments, wherein the implantable medical device comprises an active portion configured to monitor, treat or perform a function of a body of a patient.
63. The system according to embodiment 62, wherein the active portion is not a pacemaker, hearing aid or a neurostimulation implant.
64. The system according to any one of the preceding embodiments, wherein the internal communications unit is adapted to be implanted at a tissue depth of at least 8 cm or at least 15 cm.
65. The system according to any one of the preceding embodiments, wherein the internal communications unit is adapted to be implanted in an abdomen of a patient.
66. The system according to any one of the preceding embodiments, wherein the external communications unit is configured to communicate with another external device.
67. The system according to any one of the preceding embodiments, wherein the internal communications unit is configured to encrypt data before transmitting it to the external communications unit.
68. The system according to embodiment 67, wherein the external communications unit is configured to relay the encrypted data to the another external device without decrypting it.

### Aspect 457C Controlling Energy Transfer Accumulated/PID

1. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
      a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and
   a pump (5) adapted for pumping fluid between the first and second fluid chambers (1, 2).
3. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
      a breast implant system comprising:
   at least one casing with a flexible outer shape for permanent implantation in a patient's body so
   as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and
   wherein the breast implant system is adapted to allow the lubricating fluid to be at least partly removed from the casing back into the reservoir.
4. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
      a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body, said infusion needle and drive unit being designed for implantation in a patient's body, wherein the tip end of the infusion needle is laterally movable within the body of the device for variation of an injection site.
32. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises: an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
      controlling the energy transfer based on the control signal,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A method for wireless energy transfer from an external energy source located outside the patient to an internal energy receiver located inside the patient, the internal energy receiver being connected to an implantable medical device for supplying received energy thereto, the method comprising:
   determining an accumulated amount of received energy over a time period;
   determining a current change in the received energy;
   determining a control signal reflecting the accumulated received energy and the change in the transferred or received energy;
   controlling the energy transfer based on the control signal,
   wherein the implantable medical device comprises:
   a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
51. The method according to any one of the preceding embodiments, wherein determining an accumulated amount of received energy is determined by the internal energy receiver.
52. The method according to any one of the preceding embodiments, wherein determining a current change is performed by the internal energy receiver.
53. The method according to any one of the preceding embodiments, wherein the internal energy received comprises a PID regulator for controlling the energy transfer.
54. The method according to embodiment 53, wherein the PID regulator is implemented in a microcontroller.
55. The method according to any one of the preceding embodiments, wherein determining a control signal is performed by the internal energy receiver.
56. The method according to embodiment 55, wherein the control signal is transmitted to the external energy source, and wherein the external energy source is configured to adjust the transmitted energy based on the control signal.
57. The method according to any one of the preceding embodiments, wherein controlling the energy transfer is controlled by the internal energy receiver.
58. The method according to any one of the preceding embodiments, wherein controlling the energy transfer is performed by the external energy source.
59. The method according to any one of the preceding embodiments, wherein controlling the energy transfer comprises adjusting the energy transfer efficiency.
60. The method according any one of the preceding embodiments, wherein the external device comprises a transmitter coil for modulating a magnetic field for transmitting data or transmitting energy, and wherein the implantable medical device comprises a receiving coil and an NFMI receiver connected to the receiving coil to receive the data or the energy.
61. The method according to any one of the preceding embodiments, wherein at least one of the first wireless communication unit and the second wireless communication unit is configured to wirelessly charge the implantable medical device using near-field magnetic induction.
62. The method according to embodiment 61, wherein the implantable medical device comprises a coil for receiving wireless energy for charging the implant via near-field magnetic induction.
63. The method according to any one of the preceding embodiments, further comprising: receiving energy in pulses according to a pulse pattern, and measuring the received pulse pattern.
64. The method according to embodiment 63, further comprising: determining that the pulse pattern deviates from a predefined pulse pattern, and controlling the energy transfer based on the determination.
65. The method according to any one of the preceding embodiments, further comprising: measuring a temperature in the implantable medical device or in the body of the patient, and controlling the energy transfer in response to the measured temperature.
66. The method according to any one of the preceding embodiments, wherein the implantable medical device comprises at least one coil connected to a variable impedance, the method further comprising controlling the energy transfer by controlling the variable impedance.
67. The method according to any one of the preceding embodiments, wherein the implantable medical device comprises at least one coil having a plurality of windings, wherein the plurality of windings each are connected to a respective variable impedance, the method further comprising controlling the energy transfer by controlling the respective variable impedance individually.

### Aspect 459C Resonant Circuit

1. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and
   a pump (5) adapted for pumping fluid between the first and second fluid chambers (1, 2).
3. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavemosa, when the drug delivery device is implanted, sand wherein the drug delivery device is further adapted to deliver drugs through the infusion needle into the corpus cavernosum, whenever the needle is advanced into the corpus cavernosum.
31. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body, said infusion needle and drive unit being designed for implantation in a patient's body, wherein the tip end of the infusion needle is laterally movable within the body of the device for variation of an injection site.
32. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises: an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
   wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. An implantable medical device adapted to receive transcutaneously and wirelessly transmitted energy, the implantable medical device comprising:
   an energy consuming part,
   a first energy receiving unit, comprising a first coil configured for receiving transcutaneously transferred energy, and a first impedance unit electrically connected to the first coil, the receiving unit being configured to transfer the received energy to the energy consuming part
   a second energy receiving unit, comprising a second coil configured for receiving transcutaneously transferred energy and a second impedance unit electrically connected to the second coil, the receiving unit being configured to transfer the received energy to the energy consuming part,
   a measurement unit configured to measure a parameter related to energy transfer, and
   a controller configured to control the subcutaneously received energy based on the parameter by controlling the first or the second impedance unit,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
51. The implantable medical device according to any one of the preceding embodiments, wherein the first energy receiving unit has a first resonant frequency based on the inductance of the first coil and the impedance of the first impedance unit, and the second energy receiving unit has a second resonant frequency based on the inductance of the second coil and the impedance of second impedance unit.
52. The implantable medical device according to embodiment 51, wherein the first receiving unit has a resonant frequency different from the resonant frequency of the second receiving unit.
53. The implantable medical device according to any preceding embodiment, wherein the first and second impedance units are connected in parallel to the respective coil. Aspect 447AC Data_packet_encryption-Implant
1. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and a flow control device (4) configured to control the electrical stimulation device (3) to electrically stimulate the first and second portion of the wall of the uterine tube in succession for influencing the flow of sperms in the uterine tube to control pregnancy.
20. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a filtering device for removing particles from a bodily fluid of a patient, the filtering device being implantable in the patient's body and comprising: a tube forming a main fluid passageway for bodily fluid, through which the bodily fluid of the patient passes when the tube is implanted in the patient, a filter connected to the tube and, when in use, arranged for filtering the bodily fluid passing through said main fluid passageway and collecting said particles to be removed passing through the tube, and a filter cleaning device for cleaning the filter, the filter cleaning device being in contact with the filter and configured to remove particles from the filter via a movement of the filter cleaning device, and an implantable chamber arranged and adapted for receiving the removed particles from said filter cleaning device via the movement of the filter cleaning device, wherein the filter comprises a plurality of openings configured to allow the bodily fluid to pass through the filter.
22. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a filtering device for removing particles from a fluid of a patient, the filtering device being implantable in the patient's body and comprising:
   a tube forming a fluid passageway, and an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an apparatus for moving a fluid to or from a cavity in a body of a human or mammal patient, comprising:
   a fluid movement device;
   an energy source adapted to supply energy to said fluid movement device; at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area; the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body, said infusion needle and drive unit being designed for implantation in a patient's body, wherein the tip end of the infusion needle is laterally movable within the body of the device for variation of an injection site.
32. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
an infusion device implantable in a patient's body, the infusion device comprising:
   a body implantable in the patient's body and having an outer wall, an infusion needle disposed within the body of the infusion device and having a tip end, and a drive unit coupled to the infusion needle which advances and retracts the tip end of the infusion needle to penetrate any fibrosis surrounding the infusion device, when the infusion device is implanted in the patient's body, said infusion needle and drive unit being implantable in the patient's body, the drive unit being operable when implanted in the patient's body, wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gearbox, and wherein the control device comprises a control unit for controlling the gear box.
41. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a blood clot removal device for removing blood clots from the vascular system of a patient, the blood clot removal device being adapted to be implantable in the patient's body and comprising: a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway, a cleaning device (16) for moving blood clots collected by the first filter out of the blood flow passageway, and at least one second filter, wherein the cleaning device is adapted to replace the first filter in the blood flow passageway together with collected blood clots with the second filter.
49. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and a cleaning device for cleaning the filter, wherein the cleaning device is adapted to move from the first side wall towards the second side wall to move blood clots away from the blood flow passageway.
50. An implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions,
   wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and a mechanical filter cleaning device in contact with the filter, wherein said mechanical filter cleaning device is configured to: remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 447BC Data_Packet_Encryption—External Device

1. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and
   a pump (5) adapted for pumping fluid between the first and second fluid chambers (1, 2).
3. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and
   an implantable operation device (6, 8; 10; 12; 10, 14; 76-86) for operating the adjustment device, characterized in that the operation device comprises a powered operation device.
8. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device, wherein the filter comprises a plurality of openings configured to allow the bodily fluid to pass through the filter.
22. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement, wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area; the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body, said infusion needle and drive unit being designed for implantation in a patient's body, wherein the tip end of the infusion needle is laterally movable within the body of the device for variation of an injection site.
32. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
   wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder; a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member, wherein the force of the powered member exerted on the urinary bladder is at least partly exerted against the support structure when discharging urine from the urinary bladder.
42. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume, an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel, a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises:
   a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and a cleaning device for cleaning the filter, wherein the cleaning device is adapted to move from the first side wall towards the second side wall to move blood clots away from the blood flow passageway.
50. An external system for providing remote instructions to an implantable medical device, the external system being configured to:
   provide instructions to be transmitted to the implantable medical device,
      derive a checksum from the instructions,
      electronically sign the instructions and the checksum, form a data packet from the instructions, the electronic signature and the checksum, wherein the external system comprises a wireless transmitter configured to wirelessly send the data packet to the implantable medical device,
   wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 447CC Data_Packet_Encryption—Method

1. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A method of providing remote instructions from an external system to an implantable medical device, the method comprising:
   deriving a checksum, at the external system, from the instructions to be sent to the implantable medical device,
      electronically signing the instructions and the checksum, at the external system, wherein: the instructions, the checksum and the electronic signature form a data packet, wirelessly sending the data packet to the implantable medical device,
   verifying the electronic signature, and
   using the checksum to verify the integrity of the instructions,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and
   a pump (5) adapted for pumping fluid between the first and second fluid chambers (1, 2).
3. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A method of providing remote instructions from an external system to an implantable medical device, the method comprising:
   deriving a checksum, at the external system, from the instructions to be sent to the implantable medical device,
      electronically signing the instructions and the checksum, at the external system, wherein: the instructions, the checksum and the electronic signature form a data packet, wirelessly sending the data packet to the implantable medical device, verifying the electronic signature, and using the checksum to verify the integrity of the instructions,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A method of providing remote instructions from an external system to an implantable medical device, the method comprising:
   deriving a checksum, at the external system, from the instructions to be sent to the implantable medical device,
      electronically signing the instructions and the checksum, at the external system, wherein: the instructions, the checksum and the electronic signature form a data packet, wirelessly sending the data packet to the implantable medical device, verifying the electronic signature, and using the checksum to verify the integrity of the instructions,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A method of providing remote instructions from an external system to an implantable medical device, the method comprising:
   deriving a checksum, at the external system, from the instructions to be sent to the implantable medical device,
      electronically signing the instructions and the checksum, at the external system, wherein: the instructions, the checksum and the electronic signature form a data packet, wirelessly sending the data packet to the implantable medical device, verifying the electronic signature, and using the checksum to verify the integrity of the instructions,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusti ng the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and a flow control device (4) configured to control the electrical stimulation device (3) to electrically stimulate the first and second portion of the wall of the uterine tube in succession for influencing the flow of sperms in the uterine tube to control pregnancy.
20. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device, wherein the filter comprises a plurality of openings configured to allow the bodily fluid to pass through the filter.
22. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area; the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
an infusion device implantable in a patient's body, the infusion device comprising:
   a body implantable in the patient's body and having an outer wall, an infusion needle disposed within the body of the infusion device and having a tip end, and a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gearbox, and wherein the control device comprises a control unit for controlling the gear box.
41. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a blood clot removal device for removing blood clots from the vascular system of a patient, the blood clot removal device being adapted to be implantable in the patient's body and comprising: a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway, a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A method of providing remote instructions from an external system to an implantable medical device, the method comprising:
   deriving a checksum, at the external system, from the instructions to be sent to the implantable medical device,
   electronically signing the instructions and the checksum, at the external system, wherein: the instructions, the checksum and the electronic signature form a data packet,
   wirelessly sending the data packet to the implantable medical device,
   verifying the electronic signature, and
   using the checksum to verify the integrity of the instructions,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
50. The method according to any one of embodiments 1-50, further comprising the steps of encrypting the data packet at the external system using a private key of the external system, and decrypting, at the implantable medical device, the data packet using a private key of the implantable medical device.
51. The method according to any one of the preceding embodiments, wherein the step of verifying the electronic signature comprises comparing the electronic signature with electronic signatures stored in the implantable medical device.
52. The method according to any one of the preceding embodiments, wherein the step of wirelessly sending the data packet to the implantable medical device comprises sending the data packet from a first external device to a second external device using wired communication and wirelessly sending the data packet from the second external device to the implantable medical device.
53. The method according to any one of the preceding embodiments, wherein the step of wirelessly sending the data packet to the implantable medical device comprises sending the data packet from a first external device to a second external device and further wirelessly sending the data packet from the second external device to the implantable medical device, and wherein the second external device transmits the data packet without changing the data packet.
54. The method according to any one of the preceding embodiments, wherein the step of wirelessly sending the data packet to the implantable medical device comprises sending the data packet from a first external device to a second external device and further wirelessly sending the data packet from the second external device to the implantable medical device, and wherein the second external device transmits the data packet without full decryption.
55. The method according to any one of the preceding embodiments, wherein the implantable medical device comprises a control program configured to control at least one function of the implantable medical device, and wherein the method further comprises altering the control program on the basis of the received instructions.
56. The method according to any one of the preceding embodiments, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
57. The method according to claim 56, wherein the implantable medical device comprises a set of stored parameter values, and wherein the method further comprises the step of verifying that the instructions for altering the at least one parameter will result in the at least one parameter being updated to a parameter value comprised in the set of stored parameter values.

### Aspect 447DC Single-Use_Codes_Encryption

1. A method of providing remote instructions from an external system to an implantable medical device, wherein the implantable medical device comprises a list of codes and the external system comprises a list of codes, the method comprising: encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and a flow control device (4) configured to control the electrical stimulation device (3) to electrically stimulate the first and second portion of the wall of the uterine tube in succession for influencing the flow of sperms in the uterine tube to control pregnancy.
20. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device, wherein the filter comprises a plurality of openings configured to allow the bodily fluid to pass through the filter.
22. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body,
   wherein the tip end of the infusion needle is laterally movable within the body of the device for variation of an injection site.
32. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
an infusion device implantable in a patient's body, the infusion device comprising:
   a body implantable in the patient's body and having an outer wall, an infusion needle disposed within the body of the infusion device and having a tip end, and a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
   wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and
   decrypting, at the implantable medical device, the instructions using a code from a position on the list of codes,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a blood clot removal device for removing blood clots from the vascular system of a patient, the blood clot removal device being adapted to be implantable in the patient's body and comprising: a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway, a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and a cleaning device for cleaning the filter, wherein the cleaning device is adapted to move from the first side wall towards the second side wall to move blood clots away from the blood flow passageway.
50. A method of providing remote instructions from an external system to an implantable medical device, wherein the implantable medical device comprises a list of codes and the external system comprises a list of codes, the method comprising:
   encrypting the instructions at the external system using a code from a position on the list of codes,
   wirelessly sending the encrypted instructions to the implantable medical device, and decrypting, at the implantable medical device, the instructions using a code from a position on the list of codes,
   wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and a mechanical filter cleaning device in contact with the filter, wherein said mechanical filter cleaning device is configured to: remove blood clots from the filter by moving along the filter in a direction perpendicular to a side wall of the blood flow passageway to collect the blood clots at the side wall of the blood flow passageway, and
   move the removed blood clots away from the blood flow passageway.
51. The method according to any one of the preceding embodiments, further comprising the steps of: wirelessly sending position information from the external device to the implantable medical device, and using the information at the implantable medical device for selecting the code from the list of codes.
52. The method according to any one of the preceding embodiments, wherein the step of encrypting, at the external system, the instructions using a code from a position on the list of codes comprises selecting the code on a current position on the list of codes, wherein the method further comprises the step of updating the current position to a new current position after using the code.
53. The method according to any one of the preceding embodiments, wherein the step of decrypting, at the implantable medical device, the instructions using a code from a position on the list of codes comprises selecting the code on a current position on the list of codes, wherein the method further comprises the step of updating the current position to a new current position after using the code.
54. The method according to any one of claims 52 and 53, wherein the current position comprises a number and wherein the step of updating the current position comprises updating the number to a sequential number.
55. The method according to any one of the preceding embodiments, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without changing the encrypted instructions.
56. The method according to any one of the preceding embodiments, wherein the step of wirelessly sending the encrypted instructions to the implantable medical device comprises sending the encrypted instructions from a first external device to a second external device and further wirelessly sending the encrypted instructions from the second external device to the implantable medical device, and wherein the second external device transmits the encrypted instructions without full decryption.
57. The method according to any one of the preceding embodiments, wherein the implantable medical device comprises a control program configured to control at least one function of the implantable medical device, and wherein the method further comprises altering the control program on the basis of the received instructions.
58. The method according to any one of the preceding embodiments, wherein the implantable medical device comprises an internal computing unit configured to run a control program for controlling a function of the implantable medical device, wherein the control program comprises at least one adjustable parameter affecting the control of the implantable medical device, and wherein the method of providing remote instructions comprises providing instructions for altering the at least one parameter for affecting the control of the implantable medical device.
59. The method according to embodiment 58, wherein the implantable medical device comprises a set of stored parameter values, and wherein the method further comprises the step of verifying that the instructions for altering the at least one parameter will result in the at least one parameter being updated to a parameter value comprised in the set of stored parameter values.

### Aspect 331CC eHealth General Security Module

1. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
wherein the implantable medical device comprises:
a breast implant system, comprising:
a plurality of chambers including
at least one first fluid chamber (1) which is adapted to allow an increase and a decrease of its volume, and
at least one second fluid chamber (2) which is adapted to allow an increase and a decrease of its volume,
said first and second fluid chambers (1, 2) being adapted for implantation in the human body with the at least one first fluid chamber (1) forming part of a breast implant (10),
said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system comprising
an implantable medical device,
an implantable controller for the implantable medical device, the implantable controller comprises:
   a wireless transceiver for communicating wirelessly with an external device, a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system comprising
an implantable medical device,
an implantable controller for the implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or
      a combination of the response communication and the received communication from the external device,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
51. The implantable controller according to any one of the preceding embodiments, wherein the security module comprises a set of rules for accepting communication from the central unit.
52. The implantable controller according to embodiment 51, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
52. The implantable controller according to embodiment 51, wherein the set of rules comprises a rule stipulating that data from the central unit is only accepted when the wireless transceiver has been placed in the off-mode for a specific time period.
53. The implantable controller according to any one of the preceding embodiments wherein the central unit is configured to verify a digital signature of the received data from the external device and on a positive verification send the received data to the security module.
54. The implantable controller according to embodiment 52, wherein the set of rules comprises a rule stipulating that data from the central unit is only accepted when the digital signature of the received communication has been verified by the central unit.
55. The implantable controller according to any one of the preceding embodiments, wherein the central unit is configured to verify the size of the received data from the external device.
56. The implantable controller according to embodiment 55, wherein the set of rules comprises a rule stipulating that data from the central unit is only accepted when the size of the received data has been verified by the central unit.
57. The implantable controller according to any one of the preceding embodiments, wherein: the wireless transceiver is configured to receive data from the external device being encrypted with at least a first and second layer of encryption, the central unit is configured to decrypt a first layer of decryption of the data to obtain a first decrypted data, and transmit the first decrypted data comprising the second layer of encryption to the security model, and the security module is configured to decrypt the second layer of encryption of the first decrypted data and transmit the at least one instruction to the implantable medical.
58. The implantable controller according to embodiment 57, wherein the central unit is configured to decrypt a portion of the data comprising a digital signature, such that the digital signature can be verified by the central unit.

### Aspect 330C eHealth General Communication Housing

1. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An external device configured for communication with an implantable medical device implanted in a patient, the external device comprising:
   a display device,
   a housing unit configured to mechanically, disconnectably connect to the display device, the housing unit comprising: a first communication unit for receiving communication from the display device, and a second communication unit for wirelessly transmitting communication to the implantable medical device,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. An external device configured for communication with an implantable medical device implanted in a patient, the external device comprising:
   a display device,
   a housing unit configured to mechanically, disconnectably connect to the display device, the housing unit comprising:
      a first communication unit for receiving communication from the display device, and
      a second communication unit for wirelessly transmitting communication to the implantable medical device,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.
51. The external device according to any one of the preceding aspects, wherein the external device comprises a handheld electronic device.
52. The external device according to any one of the preceding aspects, wherein the external device is configured for communicating with the implantable medical device for changing an operational state of the implantable medical device.
53. The external device according to any one of the preceding aspects, wherein the first communication unit is a wireless communication unit for wireless communication with the display device.
54. The external device according to aspect 53, wherein: the first communication unit is configured to communicate wirelessly with the display device using a first communication frequency, the second communication unit is configured to communicate wirelessly with the implantable medical device (10) using a second communication frequency, and the first and second communication frequencies are different.
55. The external device according to any one of the preceding aspects, wherein the second communication unit is configured to communicate wirelessly with the implantable medical device (10) using electromagnetic waves at a frequency below 100 kHz.
56. The external device according to any one of the preceding aspects, wherein the second communication unit is configured to communicate wirelessly with the implantable medical device (10) using electromagnetic waves at a frequency below 40 kHz.
57. The external device according to any one of aspects 54-56, wherein the first communication unit is configured to communicate wirelessly with the display device using electromagnetic waves at a frequency above 100 kHz.
58. The external device according to any one of the preceding aspects, wherein: the first communication unit is configured to communicate with the display device using a first communication protocol, the second communication unit is configured to communicate wirelessly with the implantable medical device (10) using a second communication protocol, and the first and second communication protocols are different.
59. The external device according to any one of aspects 52-58, wherein the housing unit comprises: a first antenna configured for wireless communication with the display device, and a second antenna configured for wireless communication with the implantable medical device (10).

### Aspect 331SEC eHealth General Security Module

1. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of:
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m3, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall,
   wherein the inflatable volume filling device further comprises an expandable structure, adapted to expand, when the inflatable volume filling device is invaginated in the stomach wall, wherein said expandable structure comprises a bellows.
39. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. An implantable controller for an implantable medical device, the implantable controller comprises:
   a wireless transceiver for communicating wirelessly with an external device,
   a security module, and
   a central unit configured to be in communication with the wireless transceiver, the security module and the implantable medical device:
      the wireless transceiver is configured to receive communication from the external device including at least one instruction to the implantable medical device, and transmit the received communication to the central unit,
      the central unit is configured to send secure communication to the security module, derived from the received communication from the external device, and
      the security module is configured to at least one of
         decrypt at least a portion of the secure communication, and
         verify the authenticity of the secure communication, and
      the security module is configured to transmit a response communication to the central unit, and
      the central unit is configured to communicate the at least one instruction to the implantable medical device, the at least one instruction being based on:
         the response communication, or a combination of the response communication and the received communication from the external device,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 384C—eHealth_General_Communication_Dual

1. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m3, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall,
   wherein the inflatable volume filling device further comprises an expandable structure, adapted to expand, when the inflatable volume filling device is invaginated in the stomach wall, wherein said expandable structure comprises a bellows.
39. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the apparatus is wherein the force of the powered member exerted on the urinary bladder is at least partly exerted against the support structure when discharging urine from the urinary bladder.
42. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. An external device configured for communication with an implantable medical device when implanted in a patient, the external device comprising:
   at least one first wireless transceiver configured for communication with the implantable medical device using a first network protocol, for determining a distance between the external device and the implantable medical device, and
   at least one second wireless transceiver configured for communication with the implantable medical device using a second network protocol, for transferring data between the external device and the implantable medical device,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 385C—eHealth_General_General_Remote Display Portal

1. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611-619′), which is also comprised in the artificial valve and arranged to be placed external to said blood vessel.
30. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises: a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A patient external device configured for communication with an implantable medical device when implanted in a patient, the patient external device comprising:
   a wireless communication unit configured for wireless transmission of control commands to the implantable medical device and configured for wireless communication with a patient display device, and
   a computing unit configured for running a control software for creating the control commands for the operation of the implantable medical device, wherein the computing unit is configured to:
   transmit a control interface as a remote display portal to a patient display device configured to display the control interface to a user,
   receive user input from the patient display device, and
   transform the user input into the control commands for wireless transmission to the implantable medical device,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 386C—eHealth_General_General_App_in_App

1. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecalpassageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611-619′), which is also comprised in the artificial valve and arranged to be placed external to said blood vessel.
30. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
   wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m3, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall,
   wherein the inflatable volume filling device further comprises an expandable structure, adapted to expand, when the inflatable volume filling device is invaginated in the stomach wall, wherein said expandable structure comprises a bellows.
39. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A patient display device for communication with a patient external device for communication with an implantable medical device, when implanted, the patient display device comprising:
   a wireless communication unit,
   a display, and
   an input device for receiving implant control input from the user, wherein the patient display device is configured to:
   run a first application for wireless communication with a server and/or DDI, and
   run a second application for wireless communication with the patient external device for transmission of the implant control input to a remote display portal of the patient external device for the communication with the implantable medical device, wherein the second application is configured to be accessed through the first application,
   wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 387SEC—eHealth_General_Encryption_End-to-End

1. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
      a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume, said first and second fluid chambers (1, 2) being adapted for implantation in the human body with the at least one first fluid chamber (1) forming part of a breast implant (10), said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
      a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
      an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device, wherein the filter comprises a plurality of openings configured to allow the bodily fluid to pass through the filter.
22. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
      a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
      a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area; the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
an infusion device implantable in a patient's body, the infusion device comprising:
   a body implantable in the patient's body and having an outer wall, an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
      a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m3, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
      a male sexual impotence treatment prosthesis apparatus, comprising:
      an operable prosthesis implantable in the cavities of the corpora cavernosa of an impotent patient to provide erect penile condition, when the prosthesis is operated,
      an energy transmission device for wireless transmission of energy of a first form from outside the patient's body to inside the patient's body for use in connection with the operation of the prosthesis, when the prosthesis is implanted, and
      an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gearbox, and wherein the control device comprises a control unit for controlling the gear box.
41. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume, an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
   an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel, a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a blood clot removal device for removing blood clots from the vascular system of a patient, the blood clot removal device being adapted to be implantable in the patient's body and comprising: a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway, a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and a cleaning device for cleaning the filter, wherein the cleaning device is adapted to move from the first side wall towards the second side wall to move blood clots away from the blood flow passageway.
50. A communication system for enabling communication between a patient display device, a patient external device, a server and an implantable medical device, the communication system comprising:
   a server,
   a patient external device, and
   an implantable medical device,
   wherein a patient display device is adapted to co-operate with at least one of the patient external device and the server and further adapted to use:
      a wireless communication unit for wirelessly communicating with at least one of the patient external device and the server,
   a display, and
      an input device for receiving input from the user,
   wherein the patient external device comprises a wireless communication unit configured for wireless transmission of control commands to the implantable medical device and configured for wireless communication with at least one of the patient display device and the server,
   wherein the server comprises a wireless communication unit configured for wireless communication with at least one of the patient display device and the patient external device,
   wherein the implantable medical device comprises a wireless communication unit configured for wireless communication with the patient external device,
   wherein the implantable medical device comprises an encryption unit and is configured to: encrypt data destined for the server, transmit the data to the server via at least one of the patient external device and a second external device or patient EID, wherein at least one of the patient external device and the patient EID,
   wherein the implantable medical device comprises an encryption unit and is configured to: encrypt data destined for the patient display device, transmit the data to the patient display device via the patient external device, wherein the patient external device acts as a router transferring the data without full decryption, or
   wherein the server comprises an encryption unit and is configured to: encrypt data destined for the implantable medical device, transmit the data to the implantable medical device via the patient external device or patient EID, wherein the patient external device or patient EID acts as a router transferring the data authenticated and with or without full decryption, or
   wherein the server comprises an encryption unit and is configured to: encrypt data destined for the implantable medical device, transmit the data to the implantable medical device via the patient display device and the patient external device, wherein the patient display device and the patient external device acts as a router transferring the data with or without full decryption, or
   wherein the patient display device comprises an encryption unit and is configured to: encrypt data destined for the implantable medical device, transmit the data to the implantable medical device via the patient external device, wherein the patient external device acts as a router transferring the data with or without full decryption, or
   wherein the patient display device comprises an encryption unit and is configured to: encrypt data destined for the implantable medical device, transmit the data to the implantable medical device via the server and the patient external device or patient EID, wherein the server and the patient external device or the patient EID acts as a router transferring the data with or without full decryption,
   wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and a mechanical filter cleaning device in contact with the filter, wherein said mechanical filter cleaning device is configured to: remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 396C—eHealth_General_Communication_Dual

1. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured for changing pre-programmed treatment settings of an implantable medical device, when implanted in a patient, from a distant remote location in relation to the patient, the system comprising:
   at least one health care provider, HCP, EID external device, and a HCP private key device, wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication; wherein said HCP EID external device comprises at least one of a reading slot or comparable for the HCP private key device,
   a RFID communication, and other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
      an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement, wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area; the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
an infusion device implantable in a patient's body, the infusion device comprising:
   a body implantable in the patient's body and having an outer wall, an infusion needle disposed within the body of the infusion device and having a tip end, and a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises: a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel, a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises: a blood clot removal device for removing blood clots from the vascular system of a patient, the blood clot removal device being adapted to be implantable in the patient's body and comprising: a blood flow passageway (14) being adapted be connected to the patient's vascular system to allow
   circulation of the patient's blood through the blood flow passageway, a first filter (12) provided in the blood flow passageway for collecting blood clots occurring in the blood flowing through the blood flow passageway, a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of: a reading slot or comparable for the HCP private key device, and a RFID communication or other close distance wireless activation communication;
wherein said HCP EID external device comprises at least one of:
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured for changing pre-programmed treatment settings of an implantable medical device, when implanted in a patient, from a distant remote location in relation to the patient, the system comprising:
   at least one health care provider, HCP, EID external device, and
   a HCP private key device,
wherein the HCP EID external device is adapted to receive a command from the HCP to change said pre-programmed treatment settings of an implanted medical device, and further adapted to be activated and authenticated and allowed to perform said command by the HCP providing the HCP private key device, wherein the HCP private key device is adapted to be provided to the HCP EID external device via at least one of a reading slot or comparable for the HCP private key device, and a RFID communication or other close distance wireless activation communication;
wherein said HCP EID external device comprises at least one of
   a reading slot or comparable for the HCP private key device,
   a RFID communication, and
   other close distance wireless activation communication or electrical direct contact;
wherein said HCP EID external device further comprises at least one wireless transceiver configured for communication with a data infrastructure server, DDI, through a first network protocol,
wherein said system further comprises:
   a data infrastructure server, DDI, adapted to receive command from said HCP EID external device and to relay the received command without modifying said command to a patient EID external device, wherein the DDI comprises one wireless transceiver configured for communication with said patient external device, and
   a patient EID external device adapted to receive the command from the HCP EID relayed by the DDI, further adapted to send this command to the implanted medical device, said command adapted to change said pre-programmed treatment settings of the implanted medical device, and further adapted to be activated and authenticated and allowed to perform said command by the implant, wherein the implanted medical device is configured to treat the patient or perform a bodily function,
wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and a mechanical filter cleaning device in contact with the filter, wherein said mechanical filter cleaning device is configured to: remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 397SEC—eHealth_General_Communication_Dual

1. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted, rein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of
   a smart card,
   a keyring device,
   a watch,
   an arm or wrist band,
   a necklace, and
   any shaped device;
   wherein the HCP EID external device is adapted to be involved in at least one of receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured for changing pre-programmed treatment settings of an implantable medical device, when implanted in a patient, by a health care provider, HCP, in the physical presence of the patient, the system comprising:
   at least one HCP EID external device adapted to receive a command from the HCP, directly or indirectly, to change said pre-programmed treatment settings in steps of an implantable medical device, when implanted, wherein the HCP EID external device is further adapted to be activated, authenticated, and allowed to perform said command by the HCP providing a HCP private key device comprising a HCP private key,
   wherein the HCP private key device comprises at least one of:
   a smart card,
   a keyring device,
   a watch,
   an arm or wrist band,
   a necklace, and
   any shaped device;
   wherein the HCP EID external device is adapted to be involved in at least one of: receiving information from the implant, receiving information from a patient EID, receiving information direct or indirect from a remote external device, actuating the implanted medical device, changing pre-programmed settings, and updating software of the implantable medical device, when implanted,
   wherein the HCP EID external device is adapted to be activated, authenticated, and allowed to perform said command also by the patient,
   wherein the system further comprises:
   a patient private key device comprising a patient private key, the patient private key device comprising at least one of:
   a smart card,
   a keyring device,
   a watch,
   an arm or wrist band,
   a necklace, and
   any shaped device;
   wherein the HCP private key and the patient private key are required for performing said actions by the HCP EID external device to at least one of: receive information from the implant, to receive information direct or indirect from a patient remote external device, to actuate the implanted medical device, to change pre-programmed settings, and to update software of the implantable medical device, when the implantable medical device is implanted,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 398SEC—eHealth_General_Communication_Dual

1. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured to change pre-programmed and pre-selected treatment actions of an implantable medical device, when implanted in a patient, by command from the patient, thesystem comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured to change pre-programmed and pre-selected treatment actions of an implantable medical device, when implanted in a patient, by command from the patient, the system comprising:
   an implantable medical device, a patient remote external device, comprising a wireless transceiver configured for communication with the implantable medical device, when the medical device is implanted, and a remote display portal configured to receive content delivered from the patient remote external device to expose buttons to express the will to actuate the functions of the implanted medical device by the patient through the patient remote external device, and further configured to present the display portal remotely on a patient display device allowing the patient to actuate the functions of the implanted medical device through the display portal of the patient remote external device visualized on the patient display device,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 399SEC—eHealth_General_implant information

1. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key, a patient private key device comprising the private key adapted to be provided to the patient EID external device via at least one of: a reading slot or comparable for the patient private key device, a RFID communication or other close distance wireless activation communication or direct electrical connection, wherein said patient EID external device comprises at least one of; a reading slot or comparable for the patient private key device, an RFID communication, and other close distance wireless activation communication or direct electrical contact; wherein said patient EID external device further comprises at least one wireless transceiver configured for communication with the DDI, through a first network protocol,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key, a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of; a reading slot or comparable for the patient private key device, an RFID communication, and other close distance wireless activation communication or direct electrical contact; wherein said patient EID external device further comprises at least one wireless transceiver configured for communication with the DDI, through a first network protocol,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key, a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of; a reading slot or comparable for the patient private key device, an RFID communication, and other close distance wireless activation communication or direct electrical contact; wherein said patient EID external device further comprises at least one wireless transceiver configured for communication with the DDI, through a first network protocol,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection, wherein said patient EID external device comprises at least one of: a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611-619′), which is also comprised in the artificial valve and arranged to be placed external to said blood vessel.
30. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection, wherein said patient EID external device comprises at least one of: a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured for providing information from an implantable medical device, when implanted in a patient, to a distant remote location in relation to the patient, the system comprising:
   at least one patient EID external device 320′″ adapted to receive information from the implant, adapted to send such information further on to a server or dedicated data infrastructure, DDI, 330, further adapted to be activated and authenticated and allowed to receive said information by the implanted medical device by the patient providing a private key,
   a patient private key device comprising the private key adapted to be provided to the patient EID external device via at least one of: a reading slot or comparable for the patient private key device, a RFID communication or other close distance wireless activation communication or direct electrical connection,
   wherein said patient EID external device comprises at least one of;
   a reading slot or comparable for the patient private key device,
   an RFID communication, and
   other close distance wireless activation communication or direct electrical contact;
   wherein said patient EID external device further comprises at least one wireless transceiver configured for communication with the DDI, through a first network protocol,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 400C—eHealth_General_system, DDI inactivation of remote or private key

1. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611-619′), which is also comprised in the artificial valve and arranged to be placed external to said blood vessel.
30. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system comprising, an implantable medical device adapted to, when implanted in a patient, to communicate with an external device, the external device comprising at least one of a patient remote external device or a patient EID external device, the system further comprising: the patient EID external device adapted to communicate with and send commands to the implantable medical device when implanted, to change pre-programmed settings, a patient private key device comprising a patient private key, adapted to activate and authenticate and allow to perform said command by the patient EID external device, wherein said private key is adapted to be provided to the external device via at least one of: a reading slot or comparable for the HCP private key device, an RFID communication or other close distance wireless activation communication, or direct electrical contact, and a data infrastructure server, DDI, adapted to send commands to the patient EID external device for further transport to the implanted medical device, to inactivate the authority and authenticating function of the patient private key,
wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and a mechanical filter cleaning device in contact with the filter, wherein said mechanical filter cleaning device is configured to: remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect 401C—eHealth_General_patient_at hospital

1. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured for changing pre-programmed treatment settings in steps of an implantable medical device, when implanted in a patient, by a health care provider, HCP, either in the physical presence of the patient or remotely with the patient on distance, the system comprising:
   at least one HCP EID external device adapted to receive a command directly or indirectly from the HCP to change said pre-programmed treatment settings in steps of the implantable medical device, when implanted, wherein the HCP EID external device is further adapted to be activated, authenticated, and allowed to perform said command by the HCP providing: a HCP private key device comprising a HCP private key, comprising at least one of:
   a smart card,
   a keyring device,
   a watch,
   an arm or wrist band,
   a necklace, and
   any shaped device; wherein the system further comprises: a patient private key device comprising a patient private key, comprising at least one of:
   a smart card,
   a keyring device,
   a watch,
   an arm or wrist band,
   a necklace, and
   any shaped device; wherein both the HCP and patient private key is required for performing said action by the HCP EID external device to change the pre-programmed settings in the implant and to update software of the implantable medical device, when the implantable medical device is implanted, and wherein the patient private key is adapted to activate, be authenticated, and allowed to perform said command provided by the HCP, either via the HCP EID external device or when the action is performed remotely via a patient EID external device,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway. Aspect 402C—eHealth_General_EID and DDI
1. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and
   the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube,
   wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an apparatus for drainage of a body fluid in a human or mammal patient, comprising:
   a drainage device,
   an energy source adapted to supply energy to said drainage device, and
   at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein:
   the fluid movement device and the at least one connecting tube form a fluid moving arrangement adapted to be implanted inside the body of the human or mammal patient;
   the tube end is adapted to be inserted into an organ of the patient;
   at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises: an infusion device, comprising
   a body
   an infusion needle having a tip end, and
   a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   an implantable infusion device, comprising:
   an infusion needle having a tip end,
   a reservoir coupled to the infusion needle, and
   a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m3, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and
   an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and a control device for controlling an operation of the powered member,
   wherein the force of the powered member exerted on the urinary bladder is at least partly exerted against the support structure when discharging urine from the urinary bladder.
43. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
an apparatus for treating urinary retention of a patient by assisting the discharging of urine from a natural urinary bladder of a patient, comprising:
   an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and
   a cleaning device for cleaning the filter, wherein the cleaning device is adapted to move from the first side wall towards the second side wall to move blood clots away from the blood flow passageway.
50. A system configured for changing pre-programmed treatment settings in steps of an implantable medical device, when implanted in a patient, by a health care provider, HCP, with the patient on remote on distance, the system comprising:
   at least one HCP EID external device adapted to receive a command from the HCP direct or indirect, to change said pre-programmed treatment settings in steps of an implantable medical device, when implanted, wherein the HCP EID external device is further adapted to be activated, authenticated, and allowed to perform said command by the HCP; wherein said action by the HCP EID external device to change pre-programmed settings in the implant and to update software of the implantable medical device, when the implantable medical device is implanted, is adapted to be authenticated by a HCP private key device and a patient private key device,
wherein the implantable medical device comprises:
a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and
   move the removed blood clots away from the blood flow passageway.

### Aspect Large coil C

1. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a device for bone adjustment in a mammal, the device comprising:
   a bone adjusting housing, sized and adapted to be implanted entirely intramedullary in a femoral or tibial bone in said mammal said housing comprising
   two or more anchoring devices adapted to be attached to the femoral or tibial bone in said mammal,
   an adjustment device for exerting a force on said two or more anchoring devices, said adjustment device being adapted to postoperatively adjust at least one of a distance between or orientation of, at least two of said two or more anchoring devices,
   an operation device comprising a motor, wherein said operation device is adapted to operate said adjustment device,
   a gearbox connected to the motor, wherein an outgoing speed from the gear box is lower than a speed by the motor alone, accomplished by the gearbox, and wherein the outgoing speed of the gearbox is less than 10 turn per second during normal operation, and wherein said device for bone adjustments further comprises
   a control device adapted to control the operation device, wherein the control device is programmed to control the device according to a pre-programmed time-schedule comprising a program of incremental changes.
2. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a breast implant system, comprising:
   a plurality of chambers including
   at least one first fluid chamber (1) which is adapted to allow an increase and a decrease of its volume, and
   at least one second fluid chamber (2) which is adapted to allow an increase and a decrease of its volume,
   said first and second fluid chambers (1, 2) being adapted for implantation in the human body with the at least one first fluid chamber (1) forming part of a breast implant (10),
   said fluid chambers (1, 2) being interconnected when implanted such that fluid can be exchanged between the first and second fluid chambers (1, 2) so as to change their respective fluid content, and a pump (5) adapted for pumping fluid between the first and second fluid chambers (1, 2).
3. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a breast implant system comprising:
   at least one casing with a flexible outer shape for permanent implantation in a patient's body so as to form part of a breast implant,
   at least one first element contained in the casing, and
   a reservoir adapted to be implanted and filled with a lubricating fluid, the reservoir being connected to the casing so as to allow the lubricating fluid to be supplied from the reservoir to the casing between an outer surface of said at least one first element contained in the casing and surfaces contacting said outer surface in order to reduce surface friction between the outer surface of said at least one first element and the surfaces contacting said outer surface, and wherein the breast implant system is adapted to allow the lubricating fluid to be at least partly removed from the casing back into the reservoir.
4. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a breast implant system for implantation in a patient's body so as to form part of a breast implant, comprising at least one first element and at least one second element, each of the first and second elements comprising an outer wall defining a volume which is filled or fillable with a filling material, wherein the first element is displaceable relative to the second element by allowing the first element to be moved, when implanted in the patient's breast, between different spots within the patient's breast so as to change the outer shape of the breast implant, and wherein the breast implant system further comprises at least one reservoir in fluid connection with the at least one first element, such that fluid can be exchanged between the reservoir and the at least one first element.
5. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an anal incontinence treatment apparatus, comprising: an energy transmission means for wireless transmission of energy of a first form from outside a patient's body; an operable restriction device implantable in a patient for engaging the colon or rectum to form a restricted passageway in the colon or rectum, the device operable in response to energy of a second form different than the energy of the first form to vary the restricted passageway; and an energy transforming means implantable in the patient for transforming energy of the first form wirelessly transmitted by the energy transmission means into energy of the second form, which is used for the operation of the restriction device.
6. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: an anal incontinence disease treatment apparatus for treatment of a patient, who suffers from anal incontinence, comprising a restriction device implantable in the patient for engaging the colon, rectum or anus to restrict a fecal passageway therein, the restriction device being operable to change the restriction of the fecal passageway, characterized by an energy transmission device for wireless transmission of energy from outside the patient's body to inside the patient's body for use in connection with the operation of the restriction device including to change the restriction of the fecal passageway, when the restriction device is implanted.
7. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   anal incontinence treatment apparatus, comprising an adjustable restriction device (2) adapted to engage the colon, rectum or anus of a patient, who suffers from anal incontinence, to form a restricted fecal passageway in the colon, rectum or anus, an implantable adjustment device (5) for adjusting the restriction device (2) to restrict the colon, rectum or anus to close the fecal passageway, or release the colon, rectum or anus to open the fecal passageway, when the restriction device is implanted in the patient, and an implantable operation device (6, 8; 10; 12; 10, 14; 76-86) for operating the adjustment device, characterized in that the operation device comprises a powered operation device.
8. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an anal incontinence treatment apparatus for treatment of a patient, who suffers from anal incontinence, comprising an adjustable restriction device implantable in the patient for engaging a portion of the colon or rectum of the patient to restrict a fecal passageway therein, and an operable adjustment device implantable in the patient for adjusting the restriction device to change the restriction of the fecal passageway, wherein the adjustment device is adapted to mechanically adjust the restriction device.
9. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an anal incontinence treatment apparatus, comprising a restriction device implantable in a patient suffering from anal incontinence for engaging the colon or rectum to form a restricted fecal passageway in the colon or rectum, the restriction device being operable to change the restriction of the fecal passageway, wherein a source of energy is provided, and a control device operable from outside the patient's body is provided for controlling the source of energy to release energy for use in connection with the operation of the restriction device, when the restriction device is implanted.
10. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment apparatus, comprising an operable restriction device implantable in an impotent patient for engaging a tissue portion of the normal penile tissue or the prolongation thereof to form a restrictable penile exit blood flow passageway, and an energy transmission device for wireless transmission of energy from outside the patient's body to inside the patient's body for use in connection with the operation of the restriction device, including temporarily contracting said tissue portion to restrict the penile exit blood flow in the blood flow passageway to achieve erection.
11. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment apparatus, comprising an adjustable restriction device implantable in a male patient, who suffers from sexual impotence, for engaging the corpora cavernosa or crura or the prolongations thereof of the patient's penile tissue, an implantable adjustment device for adjusting the restriction device to temporarily contract the corpora cavernosa or crura or the prolongations thereof to restrict the blood flow leaving the penis, and an implantable operation device for operating the adjustment device, wherein in that the operation device comprises a powered operation device.
12. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment apparatus, comprising an adjustable restriction device implantable in a male impotent patient for directly engaging a portion of the normal penile tissue or the prolongation thereof of the patient, and an operable adjustment device implantable in the patient for adjusting the restriction device to temporarily contract said portion of the normal penile tissue or the prolongation thereof to restrict the blood flow leaving the penis, when the patient desires to achieve erection, wherein that the adjustment device is adapted to mechanically adjust the restriction device.
13. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment apparatus, comprising an operable restriction device implantable in a patient for engaging the penile tissue or the prolongation thereof to form a restricted blood flow passageway through which blood flow leaves the penis, the restriction device being operable to restrict the blood flow passageway to provide erection, wherein a source of energy is provided, and a control device operable from outside the patient's body is provided for controlling the source of energy to release energy for use in connection with the operation of the restriction device, when the restriction device is implanted.
14. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a urinary incontinence treatment apparatus, comprising a restriction device implantable in a patient, who suffers from urinary incontinence, for engaging the urethra or urine bladder, to form a restricted urine passageway in the urethra or urine bladder, the restriction device being operable to change the restriction of the urine passageway, and an energy transmission device for wireless transmission of energy from outside the patient's body to inside the patient's body for use in connection with the operation of the restriction device including enlarging or restricting the urine passageway, when the restriction device is implanted.
15. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a urinary incontinence treatment apparatus, comprising an adjustable restriction device adapted to engage the urethra or urine bladder of a patient, who suffers from urinary incontinence, to form a restricted urine passageway in the urethra or urine bladder, an implantable adjustment device for adjusting the restriction device to restrict the urethra or urine bladder to close the urine passageway, or release the urethra or urine bladder to open the urine passageway, when the restriction device is implanted in the patient, and an implantable operation device for operating the adjustment device, wherein the operation device comprises a powered operation device.
16. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a urinary incontinence treatment apparatus for treatment of a patient, who suffers from urinary incontinence, comprising an adjustable restriction device implantable in the patient for engaging a portion of the urethra or urine bladder of the patient to restrict a urine passageway therein, and an operable adjustment device adapted to mechanically adjust the restriction device to change the restriction of the urine passageway, wherein the adjustment device is inoperable by permanent, static magnetic energy.
17. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a urinary incontinence treatment apparatus, comprising a restriction device implantable in a patient suffering from urinary incontinence for engaging the urethra or urine bladder to form a restricted urine passageway in the urethra or urine bladder, the restriction device being operable to change the restriction of the urine passageway, characterized by a source of energy and a control device operable from outside the patient's body for controlling the source of energy to release energy for use in connection with the operation of the restriction device, when the restriction device is implanted.
18. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
a pregnancy control system for promoting pregnancy of a female human patient, comprising:
   an implantable restriction device (2) adapted to be postoperatively adjusted to restrict the lumen of an oviduct (31a, 31b) of the patient to accumulate at least one egg released from the ovary in the oviduct and to cease restricting the oviduct lumen to admit the egg in the oviduct to be transported to the uterus,
   the restriction device (2) is adapted to be positioned on the outside of the oviduct (31a, 31b); and the pregnancy control system further comprises a control device (32, 33) operable from outside the patient's body to control the restriction device, wherein the control device comprises an internal control unit (33) adapted to be implanted in the patient's body, the internal control unit controlling the restriction device,
   wherein the restriction device comprises at least two restriction areas adapted to restrict the oviduct lumen at different places on the oviduct, the restriction device being adapted to change the restriction of the oviduct lumen over time from one restriction area to another restriction area.
19. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a pregnancy control apparatus comprising an implantable flow influence device configured to be implanted in the body of a female human patient in connection with a uterine tube to influence a flow of sperms appearing in the lumen of the uterine tube of the human female patient, the implantable flow influence device comprising:
   an electrical stimulation device (3) configured to electrically stimulate at least a first and a second portion of a wall of the uterine tube for at least restricting the uterine tube, and
   a flow control device (4) configured to control the electrical stimulation device (3) to electrically stimulate the first and second portion of the wall of the uterine tube in succession for influencing the flow of sperms in the uterine tube to control pregnancy.
20. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a filtering device for removing particles from a fluid of a patient, the filtering device being implantable in the patient's body and comprising:
   a tube forming a fluid passageway, and
   a filter connected to the tube for filtering particles passing through the tube,
   wherein the filtering device further comprises
   a filter cleaning device comprising a first piston for mechanically cleaning the filter, said piston being movable in contact with said filter in a direction essentially perpendicular to the direction of the flow passageway and adapted to move the particles away from the fluid passageway upon movement of the first piston in a direction essentially perpendicular to the fluid passageway.
21. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, and
   a filter cleaning device for cleaning the filter, the filter cleaning device being in contact with the filter and configured to remove particles from the filter via a movement of the filter cleaning device, and
   an implantable chamber arranged and adapted for receiving the removed particles from said filter cleaning device via the movement of the filter cleaning device,
   wherein the filter comprises a plurality of openings configured to allow the bodily fluid to pass through the filter.
22. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a filtering device for removing particles from a bodily fluid of a patient, the filtering device being implantable in the patient's body and comprising:
   a tube forming a main fluid passageway for bodily fluid, through which the bodily fluid of the patient passes when the tube is implanted in the patient,
   a filter connected to the tube and, when in use, arranged for filtering the bodily fluid passing through said main fluid passageway and collecting said particles to be removed passing through the tube, wherein the filter comprises a plurality of openings configured to allow the bodily fluid to pass through the filter, and
   a filter cleaning device for cleaning the filter, the filter cleaning device being in contact with the filter and configured to remove particles from the filter via a movement of the filter cleaning device.
23. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the active filter comprising a cassette for changing the filter and comprising a revolving cylinder revolving over the fluid passage way.
24. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a filtering device for removing particles from a fluid of a patient, the filtering device being implantable in the patient's body and comprising:
   a tube forming a fluid passageway, and
   an active filter connected to the tube for filtering any particles passing through the tube, wherein said filter is adapted to move any particles away from the fluid passageway, post-operatively, when the filter being implanted in the patient's body, the device comprising a cleaning device for cleaning the filter and wherein the filter comprises a rotating member, having segments arranged to catch particles, the rotating member being arranged to move the particles to a rim of the rotating member, wherein the filtering device comprising an operation device, comprising a motor or pump.
25. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an apparatus for drainage of a body fluid in a human or mammal patient, comprising:
   a drainage device,
   an energy source adapted to supply energy to said drainage device, and
at least one connecting tube connected to said drainage device, the drainage device and the tube forming a drainage arrangement,
   wherein the drainage device is adapted to be implanted inside the body of the patient, and wherein said tube interconnects one part of the body with another part of the body and said drainage device is adapted to suck body fluid from the one part of the body via the connecting tube to the other part of the body, wherein the drainage device includes an expanding reservoir comprising:
   a bellow having an inlet with an inlet valve adapted to prevent fluid from exiting via the inlet when the bellow is compressed, and an outlet with an outlet valve adapted to prevent fluid from entering via the outlet when the bellow is expanded,
   a member adapted expand and compress the bellow, and
   a motor adapted to drive the member to expand the bellow to suck fluid and to compress the bellow and move fluid out, wherein said drainage device is adapted to continuously repeat expansion and compression of the bellow, whereby body fluid is removed from the treatment area to the delivery area.
26. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
an apparatus for moving a fluid to or from a cavity in a body of a human or mammal patient, comprising:
   a fluid movement device;
   an energy source adapted to supply energy to said fluid movement device;
   at least one connecting tube having a tube end, the at least one connecting tube being connected to the fluid movement device; and
   a flexible patch;
   wherein: the fluid movement device and the at least one connecting tube form a fluid moving arrangement adapted to be implanted inside the body of the human or mammal patient;
   the tube end is adapted to be inserted into an organ of the patient; at least a portion of the apt least one connecting tube is adapted to be invaginated in a wall of the organ so as to interconnect a treatment area of the body with a delivery area;
   the fluid movement device is adapted to move fluid from the treatment area via the at least one connecting tube to the delivery area;
   the flexible patch comprises a net structure adapted to be overgrown by human fibrotic tissue when implanted in the human or mammal patient; and
   the flexible patch is arranged at a distance from the tube end to allow the flexible patch to be attached to an outer surface of the organ when the at least one connecting tube is invaginated in the wall of the organ, wherein the flexible patch is further adapted to be arranged with a lateral extension along the outer surface of the organ so as to fixate and seal the at least one connecting tube to the organ by means of overgrowth of human fibrotic tissue.
27. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein the diaphragm contacting part is a grommet.
28. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: a medical device for assisting in the maintaining of an opening created in the thoracic diaphragm, said medical device comprises a diaphragm contacting part adapted to be placed in contact with the thoracic diaphragm and thereby assist in the maintaining of the opening created in the thoracic diaphragm, wherein said medical device further comprises a force transferring part, and wherein said force transferrin part is adapted to travel through the opening and transfer force between the abdominal side of the thoracic diaphragm and the thoracic side of the thoracic diaphragm or the pericardium, wherein said force transferring part comprises a mechanical element adapted to transfer mechanical force.
29. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an artificial valve (30, 50) for implantation in a mammal body, in or adjacent to a mammal blood vessel, the artificial valve comprising a casing (0′, O″; 61) and a closing mechanism, with at least part of said closing mechanism being a first moving part (31, 32, 33; 51) adapted to make movements relative to said casing (0′, O″; 61), and essentially perpendicular to the extension of the blood vessel, said movements being movements to assume an open and a closed position for opening and closing, respectively, the blood flow through said blood vessel, as well as to positions in between said open and closed positions, wherein the first moving part is adapted to have at least a portion of said first moving part (51) assuming a position at least partly external to said blood vessel in a non-closed position and receive energy for at least one of its movements at least in part from an energy device (801, 802, 803; 611-619, 611′-619′), which is also comprised in the artificial valve and arranged to be placed external to said blood vessel.
30. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a penis erection stimulation system, comprising a fully implantable drug delivery device for delivering a drug in relation to a penis to achieve erection of the penis, wherein the drug delivery device comprises at least one infusion needle adapted to be placed outside the corpora cavernosa in close proximity thereto, and wherein the infusion needle is adapted to be advanced into and retracted from at least one of the corpora cavernosa, when the drug delivery device is implanted, sand wherein the drug delivery device is further adapted to deliver drugs through the infusion needle into the corpus cavernosum, whenever the needle is advanced into the corpus cavernosum.
31. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: an infusion device, comprising
   a body
   an infusion needle having a tip end, and
a drive unit coupled to the infusion needle and arranged for axially advancing and retracting the tip end of the infusion needle to penetrate any fibrosis when the device is implanted in a patient's body,
   said infusion needle and drive unit being designed for implantation in a patient's body, wherein the tip end of the infusion needle is laterally movable within the body of the device for variation of an injection site.
32. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an implantable infusion device, comprising:
   an infusion needle having a tip end,
   a reservoir coupled to the infusion needle, and
a drive unit coupled to the infusion needle and arranged for advancing and retracting the tip end of the infusion needle to penetrate any fibrosis when the infusion device is implanted in a patient's body,
   wherein the infusion device is completely implantable in a patient's body.
33. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
an infusion device implantable in a patient's body, the infusion device comprising:
   a body implantable in the patient's body and having an outer wall,
   an infusion needle disposed within the body of the infusion device and having a tip end, and
   a drive unit coupled to the infusion needle which advances and retracts the tip end of the infusion needle to penetrate any fibrosis surrounding the infusion device, when the infusion device is implanted in the patient's body,
   said infusion needle and drive unit being implantable in the patient's body, the drive unit being operable when implanted in the patient's body,
   wherein the infusion needle is disposed within the body of the infusion device with the tip end of the infusion needle being arranged for penetrating a penetration area in the outer wall, the penetration area of the outer wall being made from a material which is self-sealing in respect of penetrations caused by the infusion needle, and wherein the drive unit further comprises an apparatus coupled to the infusion needle that laterally moves the tip end of the infusion needle within the body of the infusion device for variation of the injection site in the penetration area of the outer wall.
34. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
an artificial intestine section adapted to be implanted inside a patient's body, said intestine section having a first open end portion and a second open end portion in flow communication with one another, and further comprising at least one element adapted to directly or indirectly interact with intestinal contents contained in the artificial intestine section between the first and second open end portions thereof, wherein the at least one element comprises an artificial reservoir between the first and second open end portions for receiving and temporarily collecting therein intestinal contents supplied through the first open end portion, wherein at least the first open end portion is adapted to being connected to a surgically created lateral opening in a wall of the patient's intestine.
35. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a system for treating a patient having a disorder related to the patient's intestine, comprising an artificial reservoir adapted for receiving and temporarily collecting therein intestinal contents and further adapted to remain within the patient's body when emptying the reservoir, wherein the system further comprises an artificial flow control device implantable in the patient's body and adapted to control flow of the intestinal contents from the reservoir, and a passage in flow communication with the reservoir (40, 140), said passage being arranged for transferring intestinal contents to and from the reservoir (40, 140), wherein the flow control device comprises a pump (41, 110) for emptying the reservoir (40, 140).
36. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
an implantable medical device for lubrication of a synovial joint having a joint cavity, wherein said implantable device comprises a solid lubricant and an implantable feeding device, wherein said implantable feeding device is adapted to feed said solid lubricant into the joint cavity for lubricating the synovial joint, wherein the feeding device is adapted to propel the solid lubricant thereby feeding it into the joint, when implanted, wherein said implantable medical device further comprises a replaceable implantable cartridge containing said solid lubricant.
37. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an implantable lubrication device for lubricating a joint of a human or mammal patient by adding and circulating lubricating fluid, the lubrication device comprises:
   a reservoir adapted to store an added lubricating fluid, and
   a fluid connection device comprising a fluid connection,
wherein said fluid connection is:
   a. connected to said reservoir,
   b. adapted to extend through tissue of the patient,
   c. in connection with the joint when implanted, for introducing said lubricating fluid into said joint when the lubrication device is implanted in the patient's body, and
   a pump connected to said fluid connection device, said pump being adapted to establish said circulating lubricating fluid flow via said joint, and
   wherein the implantable lubrication device is adapted to establish a refillable circulating lubricating fluid flow via said joint, the implantable lubrication device further comprises,
   a refill injection port connected to said reservoir for refilling said reservoir with said added lubricating fluid, and
   said reservoir further comprises a chamber with an adjustable volume adapted to store the added lubricating fluid.
38. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
an apparatus for treating obesity of an obese patient having a stomach with a food cavity, the apparatus comprising:
   at least one inflatable volume filling device adapted to be completely invaginated by a stomach wall portion of the patient,
   wherein the inflatable volume filling device is adapted to be placed on the outside of the stomach wall with the outer surface of the inflatable volume filling device resting against the outside of the stomach wall, such that the volume of the food cavity is reduced in size by a volume substantially exceeding the volume of the inflatable volume filling device, wherein said inflatable volume filling device has a volume of less than 0.0002 m³, and
   wherein the at least one inflatable volume filling device is adapted to be completely invaginated by a stomach wall portion of the patient by suturing a first part of a stomach wall serosa towards a second, different part of a stomach wall serosa, the serosa being the outer layer of the stomach wall, wherein the inflatable volume filling device further comprises an expandable structure, adapted to expand, when the inflatable volume filling device is invaginated in the stomach wall, wherein said expandable structure comprises a bellows.
39. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment prosthesis apparatus, comprising:
   an operable prosthesis implantable in the cavities of the corpora cavernosa of an impotent patient to provide erect penile condition, when the prosthesis is operated,
   an energy transmission device for wireless transmission of energy of a first form from outside the patient's body to inside the patient's body for use in connection with the operation of the prosthesis, when the prosthesis is implanted, and an energy transforming device implantable in the patient for transforming the energy of the first form wirelessly transmitted by the energy transmission device into energy of a second form, the penile prosthesis being operable in response to the energy of the second form,
   wherein the energy transforming device includes at least one element having a positive region and a negative region, the element creates an energy field between the positive and negative regions when exposed to the energy of the first form transmitted by the energy transmission device, and the energy field produces the energy of the second form.
40. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a male sexual impotence treatment prosthesis—an operable non-inflatable penile prosthesis implantable in cavities of corpora cavernosa of an impotent patient's body to provide an erect penile condition, when the prosthesis is implanted,
   a source of energy implantable in the patient's body for supplying energy for operating the penile prosthesis,
   a control device operable from outside the patient's body for controlling a supply of energy from the source of energy, to control an erect and a flaccid state of the patient's penis, and
   an implantable operation device for operating the prosthesis with energy from the source of energy, wherein said operation device comprises a an electric motor, for operating the penile prosthesis through a transmission member, or for reversing the function of the penile prosthesis by a mechanical reversing device comprising an implantable gear box, and wherein the control device comprises a control unit for controlling the gear box.
41. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an apparatus adapted to treatment of urinary retention of a mammal patient by discharging urine from the urinary bladder, comprising:
   an implantable powered member (100) with a urinary bladder surface contact part, said powered member is adapted to exert a force from the outside on a selected part of the urinary bladder in order to discharge urine from the urinary bladder;
   a support structure adapted to support against at least one of, a bone or other human tissue, such as the pelvic bone, pubic bone or sacrum, other human tissue such as peritoneum, the abdominal or pelvic wall or the urine bladder itself; and
   a control device (200) for controlling the operation of the powered member,
   wherein the force of the powered member exerted on the urinary bladder is at least partly exerted against the support structure when discharging urine from the urinary bladder.
42. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an apparatus adapted to treatment of urinary retention of a mammal patient by discharging urine from a urinary bladder, comprising:
   an implantable powered member with a urinary bladder surface contact part, said powered member being adapted to exert a force from outside on a selected part of the urinary bladder in order to discharge urine from the urinary bladder;
   a support structure adapted to support against at least one of, a bone or other human tissue, and
   a control device for controlling an operation of the powered member,
   wherein the force of the powered member exerted on the urinary bladder is at least partly exerted against the support structure when discharging urine from the urinary bladder.
43. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   an apparatus for treating urinary retention of a patient by assisting the discharging of urine from a natural urinary bladder of a patient, comprising: an expandable member, adapted to be implanted inside the natural urinary bladder of the patient, for discharging urine from the natural urinary bladder as a result of its expansion in volume,
   an implantable control device for controlling the volume of the expandable member,
   an interconnecting device configured to connect the expandable member to the implantable control device, the interconnecting device being configured pass through the wall of the natural urinary bladder and seal against the wall of the natural urinary bladder, and
   an external energy transmission device for wireless transmission of energy from the outside of the patient's body to the inside of the patient's body configured to operate the expandable member.
44. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a device for treating an aneurysm of a human or mammal patient comprising:
   an implantable member adapted to hold fluid, the member being adapted to be placed in connection with the outside of a blood vessel having the aneurysm, and to exercise a pressure on the outside of the blood vessel having the aneurysm, wherein the device is adapted to prevent or reduce an expansion of the aneurysm by exerting said pressure on the outside of the blood vessel, and
   a pressure regulator adapted to regulate the pressure in the member
   a sensor or measuring device for sensing or measuring an expansion of the aneurysm
   a control unit adapted to control pressure adjustments based on a signal generated by the sensor and control the pressure regulator such that the provided pressure does not exceed a diastolic blood pressure of a treated patient having the diastolic blood pressure, such that the blood flow of the treated patient remains unaffected.
45. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a device for treating a vascular aneurysm of a human or mammal patient, comprising:
   an implantable member adapted to hold fluid, the implantable member being adapted to be placed against an outside of a blood vessel having the aneurysm and to exercise a pressure on the aneurysm to prevent or reduce an expansion of the aneurysm, wherein the implantable member comprises two or more segments that are arranged after each other in an axial direction of the blood vessel and individually adjustable in a radial direction of the blood vessel such that the implantable member can be adjusted to follow an outer contour of the aneurysm, and wherein said two or more segments are adapted to provide a pressure that is equal or less than the diastolic blood pressure of the human or mammal patient.
46. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a device for treating an aneurysm of a human or mammal patient, wherein the aneurysm may self-expand, leading to the aneurysm bursting with high risk for death of the human or mammal patient, the device comprising:
   an implantable member adapted to be placed in connection with the outside of a blood vessel having the aneurysm, and to exercise a pressure on the outside of the blood vessel having the aneurysm,
   a measuring device or sensor for measuring or sensing an expansion of the aneurysm, and
   a monitoring system adapted to monitor the expansion of the aneurysm based on a signal received from the measuring device or sensor.
47. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises:
   a device for monitoring an aneurysm of a human or mammal patient comprising:
   an implantable member adapted to be placed in connection with a blood vessel having an aneurysm and exert a pressure on the aneurysm of said blood vessel,
   a sensor adapted to be placed in relation to and outside a wall portion of the aneurysm for generating a signal corresponding to a parameter related to the aneurysm or the treatment of the aneurysm, and
   a control device adapted to receive input of a diastolic pressure over a time period and, during said time period, control the implantable member to exert the pressure on the aneurysm based on a signal generated by the sensor such that said pressure is equal to or less than the diastolic blood pressure of the treated patient.
48. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: a blood clot removal device for removing blood clots from the vascular system of a patient, the blood clot removal device being adapted to be implantable in the patient's body and comprising:
   a blood flow passageway (14) being adapted be connected to the patient's vascular system to allow
   circulation of the patient's blood through the blood flow passageway,
   a first filter (12) provided in the blood flow passageway for collecting blood clots occurring in the blood flowing through the blood flow passageway,
   a cleaning device (16) for moving blood clots collected by the first filter out of the blood flow passageway, and
   at least one second filter, wherein the cleaning device is adapted to replace the first filter in the blood flow passageway together with collected blood clots with the second filter.
49. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: a blood clot removal device for removing blood clots from the vascular system of a patient, the blood clot removal device being implantable in the patient's body and comprising:
   a blood flow passageway having a first side wall and a second side wall,
   a filter provided in the blood flow passageway for filtering blood clots, and a cleaning device for cleaning the filter, wherein the cleaning device is adapted to move from the first side wall towards the second side wall to move blood clots away from the blood flow passageway.
50. A system for wirelessly charging an implantable medical device, when implanted in a body of a patient, the system comprising: an internal energy receiver comprising a secondary coil, the internal energy receiver being connected to the implantable medical device; an external energy transmitter comprising a primary coil for wirelessly transmitting energy to the internal energy receiver via the secondary coil; wherein a diameter of the primary coil is larger than a diameter of the secondary coil,
wherein the implantable medical device comprises: a blood clot removal device for removing blood clots from the vascular system of a patient, the blood clot removal device being completely implantable in the patient's body and comprising:
   a blood flow passageway,
   a filter, provided in the blood flow passageway, for filtering blood clots, and
   a mechanical filter cleaning device in contact with the filter, wherein said mechanical filter cleaning device is configured to:
   remove blood clots from the filter by moving along the filter in a direction perpendicular to a side wall of the blood flow passageway to collect the blood clots at the side wall of the blood flow passageway, and move the removed blood clots away from the blood flow passageway.
51. The system according to any one of the preceding embodiments, wherein the system further comprises: an internal controller connected to the internal energy receiver, for controlling the amount of energy received by the internal energy receiver.
52. The system according to any one of the preceding embodiments, wherein the internal energy receiver further comprises a measurement unit for measuring a parameter related to the implantable medical device or the body of the patient.
53. The system according to any one of the preceding embodiments, wherein the controller is configured to measure the accumulated energy received by the internal energy receiver over a period of time and to measure a current change in energy received, and to control the energy received based on the accumulated energy and the current change.
54. The system according to any one of the preceding embodiments, wherein the controlled comprises a Proportional-Integral-Derivative, PID, regulator for controlling the received energy.
55. The system according to any one of the preceding embodiments, wherein the internal energy received comprises a variable impedance.
56. The system according to embodiment 55, when depending on any one of embodiments 52-54, wherein the internal energy receiver is configured to control the resonant frequency by controlling the variable impedance.
57. The system according to embodiment 55-56, wherein the controller is configured to vary the variable impedance in response to a measured parameter deviating from a predetermined interval or exceeding a threshold value.
58. The system according to embodiment 57, wherein the parameter relates to the energy received by the coil over a time period.
59. The system according to embodiment 57 or 58, wherein the measurement unit is configured to measure a parameter related to a change in energy received by the coil.

### Aspect StomaRestore 1

1. A laparoscopic surgical method of treating a patient, comprising the steps of
introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, any Serratus or any pyramidalis, the linea alba, a tendnous intersection, and the umbilicus,
blowing in pressurized gas through the needle or trocar or surgical instrument or any device inflating the abdominal cavity,
introducing working instruments comprising the steps of,
inserting at least one first trocar into the abdominal cavity,
introducing at least one camera through the trocar,
inserting at least a second trocar into the abdominal cavity,
inserting at least one instrument preferably through the second trocar,
inserting at least a third trocar into the abdominal cavity,
inserting at least one second instrument preferably through the third trocar,
dissecting an area of the jejunum or ileum,
identifying a suitable portion for creating a reservoir from the jejunum or ileum,
bending the portion of the jejunum or ileum creating a 180° bend,
using one of the first and second instrument making two small holes in the bent area,
introducing a stapler instrument into the abdomen,
separating the two halves of the stapler instrument,
introducing the two halves of the stapler instrument through the two small holes in the intestine,
clamping the intestine between the two halves of the stapler instrument,
stapling the two intestinal halves together with at least one staple line on each side of the instrument,
cutting the two intestinal halves in between the staple lines,
closing the two small holes in the intestine with sutures or staplers,
optionally bending a second intestinal area and repeating the process at least once as disclosed in the previous 8 points to increase the size of the reservoir,
implanting a flow control device so as to permanently reside inside the patient's body and be adapted to control flow of intestinal contents from the reservoir to outside the patient's body,
placing as part of the flow control device at least one electrode in one or more grows of two stapled halves of intestine laying side by side,
covering the electrode by suturing or stapling together intestine from both halves of intestine,
placing a flow control device implantable in the patient's body and adapted to control flow of the intestinal contents from the reservoir,
placing a control device in the body for controlling the flow control device,
placing at least one of a stimulation, hydraulic, pneumatic or mechanical valve for opening and closing the intestine to control flow of intestinal contents from the reservoir through the intestine out from the body,
extracting the instruments, camera and trocar, and in relation thereto
suturing, if necessary, the abdominal wall and permanently closing the skin.
2. A surgical method of treating a patient, comprising the steps of:
cutting the patient's skin and abdominal wall,
dissecting an area of the jejunum or ileum,
identifying a suitable portion for creating a reservoir from the jejunum or ileum,
bending the portion of the jejunum or ileum creating a 180° bend,
using one of the first and second instrument making two small holes in the bent area,
introducing a stapler instrument into the abdomen,
separating the two halves of the stapler instrument,
introducing the two halves of the stapler instrument through the two small holes in the intestine,
clamping the intestine between the two halves of the stapler instrument,
stapling the two intestinal halves together with at least one staple line on each side of the instrument,
cutting the two intestinal halves in between the staple lines,
closing the two small holes in the intestine with sutures or staplers,
optionally bending a second intestinal area and repeating the process at least once as disclosed in the previous 8 points to increase the size of the reservoir,
implanting a flow control device so as to permanently reside inside the patient's body and be adapted to control flow of intestinal contents from the reservoir to outside the patient's body,
placing as part of the flow control device at least one electrode in one or more grows of two stapled halves of intestine laying side by side,
covering the electrode by suturing or stapling together intestine from both halves of intestine,
placing a control device in the body,
placing a flow control device implantable in the patient's body and adapted to control flow of the intestinal contents from the reservoir,
placing at least one of a stimulation, hydraulic, pneumatic or mechanical valve for opening and closing the intestine to control flow of intestinal contents from the reservoir through the intestine out from the body,
extracting the instruments, and in relation thereto,
suturing, the abdominal wall and closing the skin
3. The method according to embodiment 1 or 2, wherein the control device comprises:
   a wireless transceiver for communicating wirelessly with an external device,
   a security module, and
   a central unit configured to be in communication with the wireless transceiver, the security module and the flow control device:
      the wireless transceiver is configured to receive communication from the external device including at least one instruction to the flow control device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the flow control device, the at least one instruction being based on:
         the response communication, or
      a combination of the response communication and the received communication from the external device.
4. The method of treating a patient according to embodiment 1 or 2, wherein the security module comprises a set of rules for accepting communication from the central unit.
5. The method of treating a patient according to embodiment 1 or 2, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
6. The method of treating a patient according to embodiment 1 or 2, wherein the set of rules comprises a rule stipulating that communication from the central unit is only accepted when the wireless transceiver has been placed in the off-mode for a specific time period.
7. The method of treating a patient according to embodiment 1 or 2, wherein the central unit is configured to verify a digital signature of the received communication from the external device.
8. The method of treating a patient according to embodiment 1 or 2, wherein the control device comprises an implantable controller for an implantable medical device, the implantable controller comprises:
   a wireless transceiver for communicating wirelessly with an external device,
   a security module, and a central unit configured to be in communication with the wireless transceiver and the security module.
   wherein: the wireless transceiver is configured to receive data from the external device including at least one instruction to the implantable medical device, and transmit the received communication to the central unit, the central unit is configured to send the data to the security module, derived from the received communication from the external device, and the security module is configured to: decrypt at least a portion of the data or verify the authenticity of the data, and communicate the at least one instruction to the flow control device based on a successful decryption or verification of the secure communication.
9. The method of treating a patient according to embodiment 8, wherein the security module comprises a set of rules for accepting communication from the central unit.
10. The method of treating a patient according to embodiment 8, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
11. The method of treating a patient according to embodiment 8, wherein the set of rules comprises a rule stipulating that data from the central unit is only accepted when the wireless transceiver has been placed in the off-mode for a specific time period.
12. The method of treating a patient according to embodiment 8, wherein the central unit is configured to verify a digital signature of the received data from the external device and on a positive verification send the received data to the security module.
13. The method of treating a patient according to embodiment 1 or 2, wherein the control device comprises an implantable medical device comprising a receiving unit comprising:
   at least one coil configured for receiving transcutaneously transferred energy,
   a measurement unit configured to measure a parameter related to the energy received by the coil, a variable impedance electrically connected to the coil, a switch placed between the variable impedance and the coil for switching off the electrical connection between the variable impedance and the coil, and a controller configured to: control the variable impedance for varying the impedance and thereby tune the coil based on the measured parameter, and control the switch for switching off the electrical connection between the variable impedance and the coil in response to the measured parameter exceeding a threshold value.
14. The method of treating a patient according to embodiment 13, wherein the controller is configured to vary the variable impedance in response to the measured parameter exceeding a threshold value.
15. The method of treating a patient according to embodiment 13, wherein the measurement unit is configured to measure a parameter related to the energy received by the coil over a time period.
16. The method of treating a patient according to embodiment 13, wherein the measurement unit is configured to measure a parameter related to a change in energy received by the coil.
17. The method of treating a patient according to embodiment 13, wherein the first switch is placed at a first end portion of the coil, and wherein the implantable medical device further comprises a second switch placed at a second end portion of the coil, such that the coil can be completely disconnected from other portions of the implantable medical device.
18. The method of treating a patient according to embodiment 1 or 2, wherein the control device comprises an implantable medical device comprising a receiving unit comprising:
   at least one coil configured for receiving transcutaneously transferred energy,
   a measurement unit configured to measure a parameter related to the energy received by the coil, a first switch is placed at a first end portion of the coil, a second switch placed at a second end portion of the coil, such that the coil can be completely disconnected from other portions of the implantable medical device, and
   a controller configured to control the first and second switch for completely disconnecting the coil from other portions of the implantable medical device on the basis of the measured parameter.
19. The method of treating a patient according to embodiment 18, wherein the controller is configured to control the first and second switch in response to the measured parameter exceeding a threshold value.
20. The method of treating a patient according to embodiment 18, wherein the measurement unit is configured to measure a parameter related to the energy received by the coil over a time period.
21. The method of treating a patient according to embodiment 18, wherein the measurement unit is configured to measure a parameter related to a change in energy received by the coil.
22. The method of treating a patient according to embodiment 18, wherein the receiving unit is configured to receive transcutaneously transferred energy in pulses according to a pulse pattern, and wherein the measurement unit is configured to measure a parameter related to the pulse pattern.
23. The method of treating a patient according to embodiment 1 or 2, wherein the control device comprises an implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions.
24. The method of treating a patient according to embodiment 23, wherein the computing unit is configured to decrypt the data packet.
25. The method of treating a patient according to embodiment 23, wherein the computing unit is configured to use the checksum to verify that the bit stream making up the instructions is unchanged.
26. The method of treating a patient according to embodiment 23, wherein the wireless receiver is part of a wireless transceiver.
27. The method of treating a patient according to embodiment 23, wherein the computing unit comprises a memory unit configured to store electronic signatures, and wherein the computing unit is configured to verify the electronic signature my comparing the electronic signature with the electronic signatures stored in the memory unit.
28. The method of treating a patient according to embodiment 1 or 2, wherein the control device is configured to receive remote instructions from an external system, wherein control device comprises a list of codes and the external system comprises a list of codes, the method comprising: wirelessly receiving encrypted instructions to the flow control device, and decrypting, at the flow control device, the instructions using a code from a position on the list of codes.
29. The method of treating a patient according to embodiment 28, further comprising the steps of: wirelessly sending position information from the external device to the flow control device, and using the information at the flow control device for selecting the code from the list of codes.
30. The method of treating a patient according to embodiment 28, wherein the step of encrypting, at the external system, the instructions using a code from a position on the list of codes comprises selecting the code on a current position on the list of codes, wherein the method further comprises the step of updating the current position to a new current position after using the code.
31. The method of treating a patient according to embodiment 28, wherein the step of decrypting, at the flow control device, the instructions using a code from a position on the list of codes comprises selecting the code on a current position on the list of codes, wherein the method further comprises the step of updating the current position to a new current position after using the code.
32. The method of treating a patient according to embodiment 30 or 31, wherein the current position comprises a number and wherein the step of updating the current position comprises updating the number to a sequential number.
33. The method of treating a patient according to embodiment 1 or 2, wherein the control device comprises an internal communications unit adapted to communicate with an external communications unit using near field magnetic induction.
34. The method of treating a patient according to embodiment 33, wherein: the internal communication unit comprises an internal NFMI receiver and an internal coil connected to the internal NFMI receiver, the internal NFMI receiver being configured to measure an induced voltage in the internal coil, the external communications unit comprises an external NFMI transmitter and an external coil connected to the external NFMI transmitter, and the external coil and the external NFMI transmitter are configured to modulate a magnetic field for sending data to the flow control device via the internal coil.
35. The method of treating a patient according to embodiment 34, wherein the external NFMI transmitter further comprises a capacitor for tuning the external coil and the external NFMI transmitter.
36. The method of treating a patient according to embodiment 35, wherein the internal NFMI receiver comprises a tunable resistor and capacitor tank for turning the internal coil and the internal NFMI receiver.
37. The method of treating a patient according to embodiment 33, wherein: the internal communication unit comprises an internal NFMI transmitter and an internal coil connected to the internal NFMI transmitter, the external communications unit comprises an external NFMI receiver and an external coil connected to the external NFMI receiver, the external NFMI receiver being configured to measure an induced voltage in the external coil, the internal coil and the internal NFMI transmitter are configured to modulate a magnetic field for sending data to the external communications unit via the external coil.
38. The method of treating a patient according to embodiment 1 or 2, wherein the control device is adapted to receive transcutaneously and wirelessly transmitted energy, the control device comprising: a first energy receiving unit, comprising a first coil configured for receiving transcutaneously transferred energy, and a first impedance unit electrically connected to the first coil, the receiving unit being configured to transfer the received energy to the flow control device,
   a second energy receiving unit, comprising a second coil configured for receiving transcutaneously transferred energy and a second impedance unit electrically connected to the second coil, the receiving unit being configured to transfer the received energy to the flow control device,
   a measurement unit configured to measure a parameter related to energy transfer, and
   a controller configured to control the subcutaneously received energy based on the parameter by controlling the first or the second impedance unit.
39. The method of treating a patient according to embodiment 1 or 2, wherein the control device is adapted to receive transcutaneously and wirelessly transmitted energy, the flow control device comprising: a receiving unit configured for receiving transcutaneously transferred energy and transferring the received energy to the flow control device, comprising: a first coil portion and a second coil portion, and a first impedance unit and a second impedance unit, wherein the first impedance unit is connected to the first coil portion and the second impedance unit is connected to the second coil portion,
   a measurement unit configured to measure a parameter related to energy transfer, and
   a controller configured to control the subcutaneously received energy based on the parameter by controlling the first or the second impedance unit.
40. The method of treating a patient according to embodiment 38 or 39, wherein the first impedance is connected in parallel to the first coil portion and the second impedance is connected in parallel to the second coil portion.
41. The method of treating a patient according to embodiment 38 or 39, wherein one of the first coil portion and the second coil portion are overlapping the other of the first coil portion and the second coil portion, or the first coil portion and the second coil portion are not overlapping with the other of the first coil portion and the second coil portion.
42. The method of treating a patient according to embodiment 38 or 39, wherein the first coil portion and the first impedance unit has a first resonance frequency, and the second coil portion and the second impedance unit has a second resonance frequency.
43. The method of treating a patient according to embodiment 1 or 2, wherein the control device comprises: a first receiving unit comprising: a first coil configured for receiving transcutaneously transferred energy and transferring the received energy to the flow control device, a first impedance electrically connected to the coil, a second receiving unit comprising: a second coil portion and a third coil portion configured for receiving transcutaneously transferred energy and transferring the received energy to the flow control device, and a second impedance unit and a third impedance unit, wherein the second impedance unit is connected to the second coil portion and the third impedance unit is connected to the third coil portion,
   a measurement unit configured to measure a parameter related to energy transfer, and
   a controller configured to control the subcutaneously received energy based on the parameter by controlling the first, the second or the third impedance unit.
44. The method of treating a patient according to embodiment 1 or 2, wherein the control device is configured to recieve wireless energy from an external energy source located outside the patient to an internal energy receiver located inside the patient, the internal energy receiver being connected to an flow control device for supplying received energy thereto, the control device being configured to:
   determining an accumulated amount of received energy over a time period;
   determining a current change in the received energy;
   determining a control signal reflecting the accumulated received energy and the change in the transferred or received energy;
   controlling the energy transfer based on the control signal.
45. The method of treating a patient according to embodiment 44, wherein determining an accumulated amount of received energy is determined by the internal energy receiver.
46. The method of treating a patient according to embodiment 44, wherein determining a current change is performed by the internal energy receiver.
47. The method of treating a patient according to embodiment 44, wherein the internal energy received comprises a PID regulator for controlling the energy transfer.
48. The method of treating a patient according to embodiment 44, wherein the PID regulator is implemented in a microcontroller.
49. The method of treating a patient according to embodiment 1 or 2, wherein the control device is configured to receive voice commands and is configured to: receive a first audio training phrase, when the flow control device is implanted in the body of the patient, create a transfer function, the transfer function being based on the first audio training phrase, wherein the transfer function is configured to adjust the amplitude of at least one frequency of audio received at the flow control device for enhancing audio received at the control device to facilitate detection of voice commands, receive a second audio training phrase comprising the voice command, the voice command comprising an instruction for the control of the flow control device, using the transfer function for generating an enhanced second audio training phrase, and
   associating the enhanced second audio training phrase with the instruction for the control of the flow control device.
50. The method of treating a patient according to embodiment 49, wherein adjusting the amplitude comprises at least one of filtering, cancelling and amplifying the at least one frequency.
51. The method of treating a patient according to embodiment 49, wherein at least one of the first and second audio training phrase is a spoken audio training phrase.
52. The method of treating a patient according to embodiment 51, wherein the spoken audio training phrase is spoken by the patient the implant is implanted in.
53. The method of treating a patient according to embodiment 49, wherein the first audio training phrase comprises the at least one voice command related to an instruction for the control of the flow control device.
54. The method of treating a patient according to embodiment 1 or 2, wherein the control device comprises a receiving unit comprising:
   at least one coil configured for receiving transcutaneously transferred energy,
   a measurement unit configured to measure a parameter related to the energy received by the coil, and a controller, wherein: the receiving unit is configured to receive transcutaneously transferred energy in pulses according to a pulse pattern, and the measurement unit is configured to measure a parameter related to the pulse pattern, and the controller is configured to control the receiving unit in response to the pulse pattern of the received energy deviating from a predetermined pulse pattern.
55. The method of treating a patient according to embodiment 54, further comprising at least one switch placed in series with the coil for switching of the coil, wherein the controller is configured to control the switch to switch of the coil in response to the pulse pattern of the received energy deviating from a predetermined pulse pattern.
56. The method of treating a patient according to embodiment 54, further comprising a variable impedance electrically connected to the coil, for varying the impedance and thereby tuning the coil, and wherein the controller is configured to control the variable impedance in response to the pulse pattern of the received energy deviating from a predetermined pulse pattern.
57. The method of treating a patient according to embodiment 54, wherein the measurement unit is configured to measure a parameter related to the energy received by the coil over a time period.
58. The method of treating a patient according to embodiment 54, wherein the measurement unit is configured to measure a parameter related to a change in energy received by the coil.
59. The method according to embodiment 1 or 2, comprising the additional steps of:
placing the valve close to the outlet of the intestine distal of the reservoir out from the body in one of the following positions; close to anus or close to a stoma opening,
60. The method according to embodiment 1 or 2, comprising the additional steps of:
fixating the device or intestine close to the device to pelvic bone, pelvic ligaments, muscle, fascia, bone, abdominal or pelvic wall, anal soft tissue.
61. The method according to embodiment 1 or 2, comprising the additional steps of:
placing at least two electrodes on the reservoir folds with at least 2 cm distance from each other to be able to execute a peristaltic type of emptying of the reservoir,
62. The method according to embodiment 1 or 2, comprising the additional steps of:
placing a holding device configured to hold at least two electrodes on the reservoir folds with at least 2 cm distance from each other to be able to execute a peristaltic type of emptying of the reservoir.
63. The method of using an pre-implanted flow control device defined in anyone of embodiments 1, 2, or
59-62, comprising the steps of:
transporting the power to the electrodes wirelessly or by wire from the control unit to the electrodes,
64. The method of using an pre-implanted flow control device defined in anyone of embodiments 1, 2, or 59-63, comprising the steps of:
stimulating by one or more electrical devices of the flow control device that form an electrical stimulation type pump advancing intestinal contents through the patient's intestine in a downstream direction on patient command,
65. The method of using an pre-implanted flow control device defined in anyone of embodiments 1, 2, or
59-64, comprising the steps of:
stimulating by the control device by the control device electrical stimulation to empty the reservoir via the at least one electrode simultaneously as the valve is opened,
66. The method of using an pre-implanted flow control device defined in anyone of embodiments 1, 2, or
59-65, comprising the steps of:
stimulating the electrodes on the reservoir folds with at least 1 second time in between the stimulation of the at least first and second electrode to be able to execute a peristaltic type of emptying of the reservoir,
67. The method according to anyone of embodiments 1, 2, or 59-66, comprising the steps of
training the intestinal wall at a different time point at the position of the valve by,
stimulating muscle or neural tissue of the intestine in a peristaltic way in the opposite direction of intestinal flow by at least two electrodes, and thereby,
68. The method according to anyone of embodiments 1, 2, or 59-67, comprising the steps of
training the intestinal muscle wall to an extent, that together with the valve that may be slightly released, constricting the intestine to hinder intestinal contents flowing through the patient's intestine in a downstream direction during the training execution.

### Aspect StomaRestore 2

1. A laparoscopic surgical method of treating a patient, comprising the steps of
introducing at least one of; a needle, a trocar, a tube, a tubular instrument and a surgical instrument, through the fascia of at least one of any muscles or any fibrotic intersections comprising; the rectus abdominalis, transversus abdominalis, the External Oblique, the Internal Oblique, any Serratus or any pyramidalis, the linea alba, a tendnous intersection, and the umbilicus,
blowing in pressurized gas through the needle or trocar or surgical instrument or any device inflating the abdominal cavity,
introducing working instruments comprising the steps of,
inserting at least one first trocar into the abdominal cavity,
introducing at least one camera through the trocar,
inserting at least a second trocar into the abdominal cavity,
inserting at least one instrument preferably through the second trocar,
inserting at least a third trocar into the abdominal cavity,
inserting at least one second instrument preferably through the third trocar,
dissecting an area of the jejunum or ileum,
identifying a suitable portion for creating a reservoir from the jejunum or ileum,
bending the portion of the jejunum or ileum creating a 180° bend,
using one of the first and second instrument making two small holes in the bent area,
introducing a stapler instrument into the abdomen,
separating the two halves of the stapler instrument,
introducing the two halves of the stapler instrument through the two small holes in the intestine,
clamping the intestine between the two halves of the stapler instrument,
stapling the two intestinal halves together with at least one staple line on each side of the instrument,
cutting the two intestinal halves in between the staple lines,
closing the two small holes in the intestine with sutures or staplers,
optionally bending a second intestinal area and repeating the process at least once as disclosed in the previous 8 points to increase the size of the reservoir,
implanting a flow control device so as to permanently reside inside the patient's body and be adapted to control flow of intestinal contents from the reservoir to outside the patient's body,
placing as part of the flow control device at least one electrode in one or more grows of two stapled halves of intestine laying side by side,
covering the electrode by suturing or stapling together intestine from both halves of intestine,
placing a flow control device implantable in the patient's body and adapted to control flow of the intestinal contents from the reservoir,
placing a control device in the body for controlling the flow control device,
placing at least one of a stimulation, hydraulic, pneumatic or mechanical valve for opening and closing the intestine to control flow of intestinal contents from the reservoir through the intestine out from the body,
extracting the instruments, camera and trocar, and in relation thereto suturing, if necessary, the abdominal wall and permanently closing the skin.
2. A surgical method of treating a patient, comprising the steps of:
cutting the patient's skin and abdominal wall,
dissecting an area of the jejunum or ileum,
identifying a suitable portion for creating a reservoir from the jejunum or ileum,
bending the portion of the jejunum or ileum creating a 180° bend,
using one of the first and second instrument making two small holes in the bent area,
introducing a stapler instrument into the abdomen,
separating the two halves of the stapler instrument,
introducing the two halves of the stapler instrument through the two small holes in the intestine,
clamping the intestine between the two halves of the stapler instrument,
stapling the two intestinal halves together with at least one staple line on each side of the instrument,
cutting the two intestinal halves in between the staple lines,
closing the two small holes in the intestine with sutures or staplers,
optionally bending a second intestinal area and repeating the process at least once as disclosed in the previous 8 points to increase the size of the reservoir,
implanting a flow control device so as to permanently reside inside the patient's body and be adapted to control flow of intestinal contents from the reservoir to outside the patient's body,
placing as part of the flow control device at least one electrode in one or more grows of two stapled halves of intestine laying side by side,
covering the electrode by suturing or stapling together intestine from both halves of intestine,
placing a control device in the body,
placing a flow control device implantable in the patient's body and adapted to control flow of the intestinal contents from the reservoir,
placing at least one of a stimulation, hydraulic, pneumatic or mechanical valve for opening and closing the intestine to control flow of intestinal contents from the reservoir through the intestine out from the body,
extracting the instruments, and in relation thereto,
suturing, the abdominal wall and closing the skin.
3. The method according to embodiment 1 or 2, wherein the control device comprises:
   a wireless transceiver for communicating wirelessly with an external device,
   a security module, and
   a central unit configured to be in communication with the wireless transceiver, the security module and the flow control device:
      the wireless transceiver is configured to receive communication from the external device including at least one instruction to the flow control device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the flow control device, the at least one instruction being based on:
         the response communication, or
      a combination of the response communication and the received communication from the external device.
4. The method according to embodiment 1 or 2, wherein the security module comprises a set of rules for accepting communication from the central unit.
5. The method according to embodiment 1 or 2, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
6. The method according to embodiment 1 or 2, wherein the set of rules comprises a rule stipulating that communication from the central unit is only accepted when the wireless transceiver has been placed in the off-mode for a specific time period.
7. The method according to embodiment 1 or 2, wherein the central unit is configured to verify a digital signature of the received communication from the external device.
8. The method according to embodiment 1 or 2, wherein the control device comprises an implantable controller for an implantable medical device, the implantable controller comprises:
   a wireless transceiver for communicating wirelessly with an external device,
   a security module, and a central unit configured to be in communication with the wireless transceiver and the security module.
   wherein: the wireless transceiver is configured to receive data from the external device including at least one instruction to the implantable medical device, and transmit the received communication to the central unit, the central unit is configured to send the data to the security module, derived from the received communication from the external device, and the security module is configured to: decrypt at least a portion of the data or verify the authenticity of the data, and communicate the at least one instruction to the implantable medical device based on a successful decryption or verification of the secure communication.
9. The method of treating a patient according to embodiment 8, wherein the security module comprises a set of rules
10. The method of treating a patient according to embodiment 8, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
11. The laparoscopic surgical method of treating a patient according to embodiment 8, wherein the set of rules comprises a rule stipulating that data from the central unit is only accepted when the wireless transceiver has been placed in the off-mode for a specific time period.
12. The method of treating a patient according to embodiment 8, wherein the central unit is configured to verify a digital signature of the received data from the external device and on a positive verification send the received data to the security module.
13. The method according to embodiment 1 or 2, wherein the control device comprises an implantable medical device comprising a receiving unit comprising:
   at least one coil configured for receiving transcutaneously transferred energy,
   a measurement unit configured to measure a parameter related to the energy received by the coil, a variable impedance electrically connected to the coil, a switch placed between the variable impedance and the coil for switching off the electrical connection between the variable impedance and the coil, and a controller configured to: control the variable impedance for varying the impedance and thereby tune the coil based on the measured parameter, and control the switch for switching off the electrical connection between the variable impedance and the coil in response to the measured parameter exceeding a threshold value.
14. The method of treating a patient according to embodiment 13, wherein the controller is configured to vary the variable impedance in response to the measured parameter exceeding a threshold value.
15. The method of treating a patient according to embodiment 13, wherein the measurement unit is configured to measure a parameter related to the energy received by the coil over a time period.
16. The method of treating a patient according to embodiment 13, wherein the measurement unit is configured to measure a parameter related to a change in energy received by the coil.
17. The method of treating a patient according to embodiment 13, wherein the first switch is placed at a first end portion of the coil, and wherein the implantable medical device further comprises a second switch placed at a second end portion of the coil, such that the coil can be completely disconnected from other portions of the implantable medical device.
18. The method according to embodiment 1 or 2, wherein the control device comprises an implantable medical device comprising a receiving unit comprising:
   at least one coil configured for receiving transcutaneously transferred energy,
   a measurement unit configured to measure a parameter related to the energy received by the coil, a first switch is placed at a first end portion of the coil, a second switch placed at a second end portion of the coil, such that the coil can be completely disconnected from other portions of the implantable medical device, and
   a controller configured to control the first and second switch for completely disconnecting the coil from other portions of the implantable medical device on the basis of the measured parameter.
19. The method of treating a patient according to embodiment 18, wherein the controller is configured to control the first and second switch in response to the measured parameter exceeding a threshold value.
20. The method of treating a patient according to embodiment 18, wherein the measurement unit is configured to measure a parameter related to the energy received by the coil over a time period.
21. The method of treating a patient according to embodiment 18, wherein the measurement unit is configured to measure a parameter related to a change in energy received by the coil.
22. The method of treating a patient according to embodiment 18, wherein the receiving unit is configured to receive transcutaneously transferred energy in pulses according to a pulse pattern, and wherein the measurement unit is configured to measure a parameter related to the pulse pattern.
23. The method according to embodiment 1 or 2, wherein the control device comprises an implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions.
24. The method of treating a patient according to embodiment 23, wherein the computing unit is configured to decrypt the data packet.
25. The method of treating a patient according to embodiment 23, wherein the computing unit is configured to use the checksum to verify that the bit stream making up the instructions is unchanged.
26. The method of treating a patient according to embodiment 23, wherein the wireless receiver is part of a wireless transceiver.
27. The method of treating a patient according to embodiment 23, wherein the computing unit comprises a memory unit configured to store electronic signatures, and wherein the computing unit is configured to verify the electronic signature my comparing the electronic signature with the electronic signatures stored in the memory unit.
28. The method according to embodiment 1 or 2, wherein the control device is configured to receive remote instructions from an external system, wherein control device comprises a list of codes and the external system comprises a list of codes, the method comprising: wirelessly receiving encrypted instructions to the flow control device, and decrypting, at the flow control device, the instructions using a code from a position on the list of codes.
29. The method of treating a patient according to embodiment 28, further comprising the steps of: wirelessly sending position information from the external device to the flow control device, and using the information at the flow control device for selecting the code from the list of codes.
30. The method of treating a patient according to embodiment 28, wherein the step of encrypting, at the external system, the instructions using a code from a position on the list of codes comprises selecting the code on a current position on the list of codes, wherein the method further comprises the step of updating the current position to a new current position after using the code.
31. The method of treating a patient according to embodiment 28, wherein the step of decrypting, at the flow control device, the instructions using a code from a position on the list of codes comprises selecting the code on a current position on the list of codes, wherein the method further comprises the step of updating the current position to a new current position after using the code.
32. The method of treating a patient according to embodiment 30 or 31, wherein the current position comprises a number and wherein the step of updating the current position comprises updating the number to a sequential number.
33. The method according to embodiment 1 or 2, wherein the control device comprises an internal communications unit adapted to communicate with an external communications unit using near field magnetic induction.
34. The method of treating a patient according to embodiment 33, wherein: the internal communication unit comprises an internal NFMI receiver and an internal coil connected to the internal NFMI receiver, the internal NFMI receiver being configured to measure an induced voltage in the internal coil, the external communications unit comprises an external NFMI transmitter and an external coil connected to the external NFMI transmitter, and the external coil and the external NFMI transmitter are configured to modulate a magnetic field for sending data to the flow control device via the internal coil.
35. The method of treating a patient according to embodiment 34, wherein the external NFMI transmitter further comprises a capacitor for tuning the external coil and the external NFMI transmitter.
36. The method of treating a patient according to embodiment 35, wherein the internal NFMI receiver comprises a tunable resistor and capacitor tank for turning the internal coil and the internal NFMI receiver.
37. The method of treating a patient according to embodiment 33, wherein: the internal communication unit comprises an internal NFMI transmitter and an internal coil connected to the internal NFMI transmitter, the external communications unit comprises an external NFMI receiver and an external coil connected to the external NFMI receiver, the external NFMI receiver being configured to measure an induced voltage in the external coil, the internal coil and the internal NFMI transmitter are configured to modulate a magnetic field for sending data to the external communications unit via the external coil.
38. The method according to embodiment 1 or 2, wherein the control device is adapted to receive transcutaneously and wirelessly transmitted energy, the control device comprising: a first energy receiving unit, comprising a first coil configured for receiving transcutaneously transferred energy, and a first impedance unit electrically connected to the first coil, the receiving unit being configured to transfer the received energy to flow control device,
   a second energy receiving unit, comprising a second coil configured for receiving transcutaneously transferred energy and a second impedance unit electrically connected to the second coil, the receiving unit being configured to transfer the received energy to the flow control device,
   a measurement unit configured to measure a parameter related to energy transfer, and
   a controller configured to control the subcutaneously received energy based on the parameter by controlling the first or the second impedance unit.
39. The method according to embodiment 1 or 2, wherein the control device is adapted to receive transcutaneously and wirelessly transmitted energy, the flow control device comprising: a receiving unit configured for receiving transcutaneously transferred energy and transferring the received energy to the flow control device, comprising: a first coil portion and a second coil portion, and a first impedance unit and a second impedance unit, wherein the first impedance unit is connected to the first coil portion and the second impedance unit is connected to the second coil portion,
   a measurement unit configured to measure a parameter related to energy transfer, and
   a controller configured to control the subcutaneously received energy based on the parameter by controlling the first or the second impedance unit.
40. The method of treating a patient according to embodiment 38 or 39, wherein the first impedance is connected in parallel to the first coil portion and the second impedance is connected in parallel to the second coil portion.
41. The method of treating a patient according to embodiment 38 or 39, wherein one of the first coil portion and the second coil portion are overlapping the other of the first coil portion and the second coil portion, or the first coil portion and the second coil portion are not overlapping with the other of the first coil portion and the second coil portion.
42. The method of treating a patient according to embodiment 38 or 39, wherein the first coil portion and the first impedance unit has a first resonance frequency, and the second coil portion and the second impedance unit has a second resonance frequency.
43. The method according to embodiment 1 or 2, wherein the control device comprises: a first receiving unit comprising: a first coil configured for receiving transcutaneously transferred energy and transferring the received energy to the flow control device, a first impedance electrically connected to the coil, a second receiving unit comprising: a second coil portion and a third coil portion configured for receiving transcutaneously transferred energy and transferring the received energy to the flow control device, and a second impedance unit and a third impedance unit, wherein the second impedance unit is connected to the second coil portion and the third impedance unit is connected to the third coil portion,
   a measurement unit configured to measure a parameter related to energy transfer, and
   a controller configured to control the subcutaneously received energy based on the parameter by controlling the first, the second or the third impedance unit.
44. The method according to embodiment 1 or 2, wherein the control device is configured to recieve wireless energy from an external energy source located outside the patient to an internal energy receiver located inside the patient, the internal energy receiver being connected to an implantable medical device for supplying received energy thereto, the control device being configured to:
   determining an accumulated amount of received energy over a time period;
   determining a current change in the received energy;
   determining a control signal reflecting the accumulated received energy and the change in the transferred or received energy;
   controlling the energy transfer based on the control signal.
45. The method of treating a patient according to embodiment 44, wherein determining an accumulated amount of received energy is determined by the internal energy receiver.
46. The method of treating a patient according to embodiment 44, wherein determining a current change is performed by the internal energy receiver.
47. The method of treating a patient according to embodiment 44, wherein the internal energy received comprises a PID regulator for controlling the energy transfer.
48. The method of treating a patient according to embodiment 44, wherein the PID regulator is implemented in a microcontroller.
49. The method of treating a patient according to embodiment 1 or 2, wherein the control device is configured to receive voice commands and is configured to: receive a first audio training phrase, when the flow control device is implanted in the body of the patient, create a transfer function, the transfer function being based on the first audio training phrase, wherein the transfer function is configured to adjust the amplitude of at least one frequency of audio received at the flow device for enhancing audio received at the control device to facilitate detection of voice commands, receive a second audio training phrase comprising the voice command, the voice command comprising an instruction for the control of the flow control device, using the transfer function for generating an enhanced second audio training phrase, and
   associating the enhanced second audio training phrase with the instruction for the control of the flow control device.
50. The method of treating a patient according to embodiment 49, wherein adjusting the amplitude comprises at least one of filtering, cancelling and amplifying the at least one frequency.
51. The method of treating a patient according to embodiment 49, wherein at least one of the first and second audio training phrase is a spoken audio training phrase.
52. The method of treating a patient according to embodiment 51, wherein the spoken audio training phrase is spoken by the patient the implant is implanted in.
53. The method of treating a patient according to embodiment 49, wherein the first audio training phrase comprises the at least one voice command related to an instruction for the control of the flow control device.
54. The method of treating a patient according to embodiment 1 or 2, wherein the control device comprises a receiving unit comprising:
   at least one coil configured for receiving transcutaneously transferred energy,
   a measurement unit configured to measure a parameter related to the energy received by the coil, and a controller, wherein: the receiving unit is configured to receive transcutaneously transferred energy in pulses according to a pulse pattern, and the measurement unit is configured to measure a parameter related to the pulse pattern, and the controller is configured to control the receiving unit in response to the pulse pattern of the received energy deviating from a predetermined pulse pattern.
55. The method of treating a patient according to embodiment 54, further comprising at least one switch placed in series with the coil for switching of the coil, wherein the controller is configured to control the switch to switch of the coil in response to the pulse pattern of the received energy deviating from a predetermined pulse pattern.
56. The method of treating a patient according to embodiment 54, further comprising a variable impedance electrically connected to the coil, for varying the impedance and thereby tuning the coil, and wherein the controller is configured to control the variable impedance in response to the pulse pattern of the received energy deviating from a predetermined pulse pattern.
57. The method of treating a patient according to embodiment 54, wherein the measurement unit is configured to measure a parameter related to the energy received by the coil over a time period.
58. The method of treating a patient according to embodiment 54, wherein the measurement unit is configured to measure a parameter related to a change in energy received by the coil.
59. The method according to embodiment 1 or 2, comprising the additional steps of:
placing the valve close to the outlet of the intestine distal of the reservoir out from the body in one of the following positions; close to anus or close to a stoma opening,
60. The method according to embodiment 1 or 2, comprising the additional steps of:
fixating the device or intestine close to the device to pelvic bone, pelvic ligaments, muscle, fascia, bone, abdominal or pelvic wall, anal soft tissue.
61. The method according to embodiment 1 or 2, comprising the additional steps of:
placing at least two electrodes on the reservoir folds with maximum 2 cm distance from each other to be able to execute a direct massive blow type of emptying of the reservoir,
62. The method according to embodiment 1 or 2, comprising the additional steps of:
placing a holding device configured to hold at least two electrodes on the reservoir folds with maximum 2 cm distance from each other to be able to execute a direct massive blow type of emptying of the reservoir.
63. The method of using an pre-implanted flow control device defined in anyone of embodiments 1, 2, or
59-62, comprising the steps of:
transporting the power to the electrodes wirelessly or by wire from the control unit to the electrodes,
64. The method of using an pre-implanted flow control device defined in anyone of embodiments 1, 2, or 59-63, comprising the steps of:
stimulating by one or more electrical devices of the flow control device that form an electrical stimulation type pump advancing intestinal contents through the patient's intestine in a downstream direction on patient command,
65. The method of using an pre-implanted flow control device defined in anyone of embodiments 1, 2, or
59-64, comprising the steps of:
stimulating by the control device by the control device electrical stimulation to empty the reservoir via the at least one electrode simultaneously as the valve is opened,
66. The method of using an pre-implanted flow control device defined in anyone of embodiments 1, 2, or
59-65, comprising the steps of:
stimulating the electrodes on the reservoir folds with maximum 1 second time in between the stimulation of the at least first and second electrode to be able to execute a direct massive blow type of emptying of the reservoir,
67. The method according to anyone of embodiments 1, 2, or 59-66, comprising the steps of:
training the intestinal wall at a different time point at the position of the valve by,
stimulating muscle or neural tissue of the intestine in a peristaltic way in the opposite direction of intestinal flow by at least two electrodes, and thereby,
68. The method according to anyone of embodiments 1, 2, or 59-67, comprising the steps of:
training the intestinal muscle wall to an extent, that together with the valve that may be slightly released, constricting the intestine to hinder intestinal contents flowing through the patient's intestine in a downstream direction during the training execution.

### Aspect Stoma Restore 3

1. An apparatus for treating a patient, comprising:
a flow control device adapted to permanently reside inside the patient's body and be adapted to control flow of intestinal contents from a reservoir made by patients intestine to empty to outside the patient's body, the flow control device comprising:
an electrical stimulation type pump advancing intestinal contents through the patient's intestine in a downstream direction on patient command, the reservoir created by: a selected suitable portion from the jejunum or ileum, that has been bent to create a 180° bend where an instrument has made two small holes in the bent area, wherein a stapler instrument with two separate two halves has been introduced through the two small holes in the intestine to clamp the intestines between the two halves of the stapler instrument to be stapled with at least one staple line on each side of the instrument and the two intestinal halves has been cut in between the staple lines to create a larger reservoir, wherein the two small holes in the intestine has been closed with sutures or staplers, and wherein optionally a second intestinal area has been bent to thereby repeat the process at least once as disclosed in this section to increase the size of the reservoir,
a control unit, wherein the control unit is configured to execute at least one of a peristaltic type of emptying of the reservoir,
at least one valve for opening and closing the intestine to control flow of intestinal contents from the reservoir through the intestine out from the body, the control unit is configured to:
stimulate the at least first and second electrode when placed on the reservoir folds with at least 1 second time in between the stimulation of the at least first and second electrode to be able to execute a peristaltic type of emptying of the reservoir, the flow control device further comprising:
at least two electrodes, at least first and second electrode, adapted to be placed on the reservoir folds of two stapled halves of intestine laying side by side at least 2 cm distance away from each other in one or more grows of the reservoir and adapted to be covered by suturing or stapling together intestine from both halves of the intestine, wherein the control unit is configured to execute a stimulation pattern creating a peristaltic type of emptying of the reservoir, wherein the control unit is configured to open the valve when the flow control device is emptying the reservoir.
2. The apparatus according to embodiment 1, wherein the control device comprises:
   a wireless transceiver for communicating wirelessly with an external device,
   a security module, and
   a central unit configured to be in communication with the wireless transceiver, the security module and the flow control device:
      the wireless transceiver is configured to receive communication from the external device including at least one instruction to the flow control device, and transmit the received communication to the central unit,
      the central unit is configured to send secure communication to the security module, derived from the received communication from the external device, and
      the security module is configured to at least one of: decrypt at least a portion of the secure communication, and
         verify the authenticity of the secure communication, and
      the security module is configured to transmit a response communication to the central unit, and
      the central unit is configured to communicate the at least one instruction to the flow control device, the at least one instruction being based on:
         the response communication, or
      a combination of the response communication and the received communication from the external device.
3. The apparatus according to embodiment 1, wherein the security module comprises a set of rules for accepting communication from the central unit.
4. The apparatus according to embodiment 1, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
5. The apparatus according to embodiment 1, wherein the set of rules comprises a rule stipulating that communication from the central unit is only accepted when the wireless transceiver has been placed in the off-mode for a specific time period.
6. The apparatus according to embodiment 1, wherein the central unit is configured to verify a digital signature of the received communication from the external device.
7. The apparatus according to embodiment 1, wherein the control device comprises an implantable controller for an implantable medical device, the implantable controller comprises:
   a wireless transceiver for communicating wirelessly with an external device,
   a security module, and a central unit configured to be in communication with the wireless transceiver and the security module.
   wherein: the wireless transceiver is configured to receive data from the external device including at least one instruction to the implantable medical device, and transmit the received communication to the central unit, the central unit is configured to send the data to the security module, derived from the received communication from the external device, and the security module is configured to: decrypt at least a portion of the data or verify the authenticity of the data, and communicate the at least one instruction to the implantable medical device based on a successful decryption or verification of the secure communication.
8. The apparatus for treating a patient according to embodiment 7, wherein the security module comprises a set of rules
9. The apparatus for treating a patient according to embodiment 8, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
10. The apparatus for treating a patient according to embodiment 7, wherein the set of rules comprises a rule stipulating that data from the central unit is only accepted when the wireless transceiver has been placed in the off-mode for a specific time period.
11. The apparatus for treating a patient according to embodiment 7, wherein the central unit is configured to verify a digital signature of the received data from the external device and on a positive verification send the received data to the security module.
12. The apparatus according to embodiment 1, wherein the control device comprises an implantable medical device comprising a receiving unit comprising:
   at least one coil configured for receiving transcutaneously transferred energy,
   a measurement unit configured to measure a parameter related to the energy received by the coil, a variable impedance electrically connected to the coil, a switch placed between the variable impedance and the coil for switching off the electrical connection between the variable impedance and the coil, and a controller configured to: control the variable impedance for varying the impedance and thereby tune the coil based on the measured parameter, and control the switch for switching off the electrical connection between the variable impedance and the coil in response to the measured parameter exceeding a threshold value.
13. The apparatus for treating a patient according to embodiment 12, wherein the controller is configured to vary the variable impedance in response to the measured parameter exceeding a threshold value.
14. The apparatus for treating a patient according to embodiment 12, wherein the measurement unit is configured to measure a parameter related to the energy received by the coil over a time period.
15. The apparatus for treating a patient according to embodiment 12, wherein the measurement unit is configured to measure a parameter related to a change in energy received by the coil.
16. The apparatus for f treating a patient according to embodiment 14, wherein the first switch is placed at a first end portion of the coil, and wherein the implantable medical device further comprises a second switch placed at a second end portion of the coil, such that the coil can be completely disconnected from other portions of the implantable medical device.
17. The apparatus according to embodiment 1, wherein the control device comprises an implantable medical device comprising a receiving unit comprising:
   at least one coil configured for receiving transcutaneously transferred energy,
   a measurement unit configured to measure a parameter related to the energy received by the coil, a first switch is placed at a first end portion of the coil, a second switch placed at a second end portion of the coil, such that the coil can be completely disconnected from other portions of the implantable medical device, and
   a controller configured to control the first and second switch for completely disconnecting the coil from other portions of the implantable medical device on the basis of the measured parameter.
18. The apparatus for treating a patient according to embodiment 17, wherein the controller is configured to control the first and second switch in response to the measured parameter exceeding a threshold value.
19. The apparatus for treating a patient according to embodiment 17, wherein the measurement unit is configured to measure a parameter related to the energy received by the coil over a time period.
20. The apparatus for treating a patient according to embodiment 17, wherein the measurement unit is configured to measure a parameter related to a change in energy received by the coil.
21. The apparatus for treating a patient according to embodiment 17, wherein the receiving unit is configured to receive transcutaneously transferred energy in pulses according to a pulse pattern, and wherein the measurement unit is configured to measure a parameter related to the pulse pattern.
22. The apparatus according to embodiment 1, wherein the control device comprises an implantable medical device configured to receive remote instructions from an external system, the implantable medical device comprising:
   a wireless receiver configured to receive wirelessly transmitted data packets from the external system,
   a computing unit configured to:
      verify the electronic signature, and
         use a checksum provided in the data packet to verify the integrity of the instructions.
23. The apparatus for treating a patient according to embodiment 22, wherein the computing unit is configured to decrypt the data packet.
24. The apparatus for treating a patient according to embodiment 22, wherein the computing unit is configured to use the checksum to verify that the bit stream making up the instructions is unchanged.
25. The apparatus for treating a patient according to embodiment 22, wherein the wireless receiver is part of a wireless transceiver.
26. The apparatus for treating a patient according to embodiment 22, wherein the computing unit comprises a memory unit configured to store electronic signatures, and wherein the computing unit is configured to verify the electronic signature my comparing the electronic signature with the electronic signatures stored in the memory unit.
27. The apparatus according to embodiment 1, wherein the control device is configured to receive remote instructions from an external system, wherein control device comprises a list of codes and the external system comprises a list of codes, the apparatus comprising: wirelessly receiving encrypted instructions to the flow control device, and decrypting, at the flow control device, the instructions using a code from a position on the list of codes.
28. The apparatus for treating a patient according to embodiment 27, further comprising the steps of.
wirelessly sending position information from the external device to the flow control device, and using the information at the flow control device for selecting the code from the list of codes.
29. The apparatus for treating a patient according to embodiment 27, wherein the step of encrypting, at the external system, the instructions using a code from a position on the list of codes comprises selecting the code on a current position on the list of codes, wherein the apparatus further comprises the step of updating the current position to a new current position after using the code.
30. The apparatus for treating a patient according to embodiment 27, wherein the step of decrypting, at the flow control device, the instructions using a code from a position on the list of codes comprises selecting the code on a current position on the list of codes, wherein the apparatus further comprises the step of updating the current position to a new current position after using the code.
31. The apparatus for treating a patient according to embodiment 29 or 30, wherein the current position comprises a number and wherein the step of updating the current position comprises updating the number to a sequential number.
32. The apparatus according to embodiment 1, wherein the control device comprises an internal communications unit adapted to communicate with an external communications unit using near field magnetic induction.
33. The apparatus for treating a patient according to embodiment 32, wherein: the internal communication unit comprises an internal NFMI receiver and an internal coil connected to the internal NFMI receiver, the internal NFMI receiver being configured to measure an induced voltage in the internal coil, the external communications unit comprises an external NFMI transmitter and an external coil connected to the external NFMI transmitter, and the external coil and the external NFMI transmitter are configured to modulate a magnetic field for sending data to the flow control device via the internal coil.
34. The apparatus for treating a patient according to embodiment 33, wherein the external NFMI transmitter further comprises a capacitor for tuning the external coil and the external NFMI transmitter.
35. The apparatus for treating a patient according to embodiment 34, wherein the internal NFMI receiver comprises a tunable resistor and capacitor tank for turning the internal coil and the internal NFMI receiver.
36. The apparatus for treating a patient according to embodiment 32, wherein: the internal communication unit comprises an internal NFMI transmitter and an internal coil connected to the internal NFMI transmitter, the external communications unit comprises an external NFMI receiver and an external coil connected to the external NFMI receiver, the external NFMI receiver being configured to measure an induced voltage in the external coil, the internal coil and the internal NFMI transmitter are configured to modulate a magnetic field for sending data to the external communications unit via the external coil.
37. The apparatus according to embodiment 1, wherein the control device is adapted to receive transcutaneously and wirelessly transmitted energy, the control device comprising: a first energy receiving unit, comprising a first coil configured for receiving transcutaneously transferred energy, and a first impedance unit electrically connected to the first coil, the receiving unit being configured to transfer the received energy to flow control device,
   a second energy receiving unit, comprising a second coil configured for receiving transcutaneously transferred energy and a second impedance unit electrically connected to the second coil, the receiving unit being configured to transfer the received energy to the flow control device,
   a measurement unit configured to measure a parameter related to energy transfer, and
   a controller configured to control the subcutaneously received energy based on the parameter by controlling the first or the second impedance unit.
38. The apparatus according to embodiment 1, wherein the control device is adapted to receive transcutaneously and wirelessly transmitted energy, the flow control device comprising: a receiving unit configured for receiving transcutaneously transferred energy and transferring the received energy to the flow control device, comprising: a first coil portion and a second coil portion, and a first impedance unit and a second impedance unit, wherein the first impedance unit is connected to the first coil portion and the second impedance unit is connected to the second coil portion,
   a measurement unit configured to measure a parameter related to energy transfer, and
   a controller configured to control the subcutaneously received energy based on the parameter by controlling the first or the second impedance unit.
39. The apparatus for treating a patient according to embodiment 37 or 38, wherein the first impedance is connected in parallel to the first coil portion and the second impedance is connected in parallel to the second coil portion.
40. The apparatus for treating a patient according to embodiment 37 or 38, wherein one of the first coil portion and the second coil portion are overlapping the other of the first coil portion and the second coil portion, or the first coil portion and the second coil portion are not overlapping with the other of the first coil portion and the second coil portion.
41. The apparatus for treating a patient according to embodiment 37 or 38, wherein the first coil portion and the first impedance unit has a first resonance frequency, and the second coil portion and the second impedance unit has a second resonance frequency.
42. The apparatus according to embodiment 1, wherein the control device comprises: a first receiving unit comprising: a first coil configured for receiving transcutaneously transferred energy and transferring the received energy to the flow control device, a first impedance electrically connected to the coil, a second receiving unit comprising: a second coil portion and a third coil portion configured for receiving transcutaneously transferred energy and transferring the received energy to the flow control device, and a second impedance unit and a third impedance unit, wherein the second impedance unit is connected to the second coil portion and the third impedance unit is connected to the third coil portion,
   a measurement unit configured to measure a parameter related to energy transfer, and
   a controller configured to control the subcutaneously received energy based on the parameter by controlling the first, the second or the third impedance unit.
43. The apparatus according to embodiment 1, wherein the control device is configured to recieve wireless energy from an external energy source located outside the patient to an internal energy receiver located inside the patient, the internal energy receiver being connected to an implantable medical device for supplying received energy thereto, the control device being configured to:
   determining an accumulated amount of received energy over a time period;
   determining a current change in the received energy;
   determining a control signal reflecting the accumulated received energy and the change in the transferred or received energy;
   controlling the energy transfer based on the control signal.
44. The apparatus for treating a patient according to embodiment 43, wherein determining an accumulated amount of received energy is determined by the internal energy receiver.
45. The apparatus for treating a patient according to embodiment 43, wherein determining a current change is performed by the internal energy receiver.
46. The apparatus for treating a patient according to embodiment 43, wherein the internal energy received comprises a PID regulator for controlling the energy transfer.
47. The apparatus for treating a patient according to embodiment 43, wherein the PID regulator is implemented in a microcontroller.
48. The apparatus for treating a patient according to embodiment 1, wherein the control device is configured to receive voice commands and is configured to: receive a first audio training phrase, when the flow control device is implanted in the body of the patient, create a transfer function, the transfer function being based on the first audio training phrase, wherein the transfer function is configured to adjust the amplitude of at least one frequency of audio received at the flow device for enhancing audio received at the control device to facilitate detection of voice commands, receive a second audio training phrase comprising the voice command, the voice command comprising an instruction for the control of the flow control device, using the transfer function for generating an enhanced second audio training phrase, and
   associating the enhanced second audio training phrase with the instruction for the control of the flow control device.
49. The apparatus for treating a patient according to embodiment 48, wherein adjusting the amplitude comprises at least one of: filtering, cancelling and amplifying the at least one frequency.
50. The apparatus for treating a patient according to embodiment 48, wherein at least one of the first and second audio training phrase is a spoken audio training phrase.
51. The apparatus for treating a patient according to embodiment 50, wherein the spoken audio training phrase is spoken by the patient the implant is implanted in.
52. The apparatus for treating a patient according to embodiment 48, wherein the first audio training phrase comprises the at least one voice command related to an instruction for the control of the flow control device.
53. The apparatus for treating a patient according to embodiment 1, wherein the control device comprises a receiving unit comprising:
   at least one coil configured for receiving transcutaneously transferred energy,
   a measurement unit configured to measure a parameter related to the energy received by the coil, and a controller, wherein: the receiving unit is configured to receive transcutaneously transferred energy in pulses according to a pulse pattern, and the measurement unit is configured to measure a parameter related to the pulse pattern, and the controller is configured to control the receiving unit in response to the pulse pattern of the received energy deviating from a predetermined pulse pattern.
54. The apparatus for treating a patient according to embodiment 53, further comprising at least one switch placed in series with the coil for switching of the coil, wherein the controller is configured to control the switch to switch of the coil in response to the pulse pattern of the received energy deviating from a predetermined pulse pattern.
55. The apparatus for treating a patient according to embodiment 53, further comprising a variable impedance electrically connected to the coil, for varying the impedance and thereby tuning the coil, and wherein the controller is configured to control the variable impedance in response to the pulse pattern of the received energy deviating from a predetermined pulse pattern.
56. The apparatus for treating a patient according to embodiment 53, wherein the measurement unit is configured to measure a parameter related to the energy received by the coil over a time period.
57. The apparatus for treating a patient according to embodiment 53, wherein the measurement unit is configured to measure a parameter related to a change in energy received by the coil.
58. The apparatus according to embodiment 1, wherein the control device is configured to empty the reservoir with electrical stimulation simultaneously as the valve is not stimulated and opened.
59. The apparatus according to embodiment 1 or 58, wherein the valve at least one of a stimulation, hydraulic, pneumatic, mechanical valve or any combination thereof.
60. The at least two electrodes according to anyone of embodiments 1 or 58-59, comprising a holding device adapted to be placed on the reservoir folds of two stapled halves of intestine laying side by side holding the electrodes with at least 2 cm distance away from each other to allow the peristaltic type of stimulation pattern and emptying of the reservoir.
61. The apparatus according to anyone of embodiments 1 or 58-60, wherein the control unit is configured to;
train the intestinal wall when the reservoir is not emptied.
62. The apparatus according to anyone of embodiments 1 or 58-61, wherein the control unit is configured to;
stimulate the muscle or neural tissue of the intestine in a peristaltic way in the opposite direction of intestinal flow by the at least two electrodes.
63. The apparatus according to anyone of embodiments 1 or 58-62, wherein the control unit is configured to;
training the intestinal muscle wall to an extent, that together with the valve that may be slightly released, constricting the intestine to hinder intestinal contents flowing through the patient's intestine in a downstream direction during the training execution.
64. The apparatus according to anyone of embodiments 1 or 58-63, comprising at least one sensor, configured to sense at least one of a physical parameter of the patient's body and a functional parameter of the apparatus.
65. The apparatus according to anyone of embodiments 1 or 58-64, comprising at least one of the following;
wherein the at least one sensor is configured to sense the physical parameter in the patient's body, comprising sensing at least one of the following physical parameters of the patient: a pressure measured on the outside of the reservoir, a pressure within the patient's intestine, an expansion of the reservoir, and a distension of an intestinal wall of the patient's intestine. wherein the at least one sensor is configured to sense the functional parameter of the apparatus when implanted, comprising sensing at least one of the following parameters: a pressure against a part of the system such as the valve, an electrical parameter such as voltage, current or energy balance, a position or movement of a movable part of the system, and any stimulation parameter in relation to the system.
66. The apparatus according to anyone of embodiments 1 or 58-65, configured to provide a signal when a value for the physical or functional parameter sensed is beyond a predetermined threshold value.
67. The apparatus according to anyone of embodiments 1 or 58-66, comprising a vibration generation unit for stimulating at least one of the intestinal portions forming the reservoir and the intestine closed by the valve.
68. The apparatus according to anyone of embodiments 1 or 58-67, wherein the vibration generating unit is attached to the casing, so that vibrations generated by the vibration generating unit can travel to the casing.
69. The apparatus according to anyone of embodiments 1 or 58-68, wherein the implantable vibration device (110) comprises an internal controller (CI).
70. The apparatus according to anyone of embodiments 1 or 58-69, wherein a internal controller (CI) is configured to wirelessly receive vibration control data for controlling the vibration of the implantable vibration device.
71. The apparatus according to anyone of embodiments 1 or 58-70, wherein the apparatus does not comprise any metallic parts and does not comprise any magnetic parts.
72. The apparatus according to anyone of embodiments 1 or 58-71, wherein the vibration generating unit is operated by a piezoelectric motor.
73. The apparatus according to anyone of embodiments 1 or 58-72, wherein at least one piezoelectric motor is arranged for driving a pump for pumping fluid to at least one of the valve or for supporting the emptying of the reservoir.
74. The apparatus according to anyone of embodiments 1 or 58-73, wherein the at least one piezoelectric motor comprises a piezoelectric inchworm motor.
75. The apparatus according to anyone of embodiments 1 or 58-74, wherein the at least one piezoelectric motor comprises a piezoelectric inertial motor.
76. The apparatus according to anyone of embodiments 1 or 58-75, wherein the at least one piezoelectric motor comprises a piezoelectric walk-drive motor.
77. The apparatus according to anyone of embodiments 1 or 58-76, wherein the piezoelectric motor is a linear piezoelectric motor.
78. The apparatus according to anyone of embodiments 1 or 58-77, wherein the linear piezoelectric motor operates with at least one of:
a speed in a range of 1 mm/s to 10 mm/s,
a stroke length in a range of 4 mm-30 mm, and
a force in a range of 2 N-30 N.
79. The apparatus according to anyone of embodiments 1 or 58-78, wherein the piezoelectric motor is a rotary piezoelectric motor.
80. The apparatus according to anyone of embodiments 1 or 58-79, wherein the rotary piezoelectric motor which operates with at least one of:
a rotational speed in a range of 1 mrad/s-100 mrad/s, and
a torque in a range of 100 Nmm-900 Nmm.
81. The apparatus according to anyone of embodiments 1 or 58-80, wherein the at least one piezoelectric motor comprises a piezoelectric ultrasonic motor.
82. The apparatus according to anyone of embodiments 1 or 58-81, wherein the piezoelectric ultrasonic motor is a traveling wave ultrasonic motor or a standing wave ultrasonic motor.
83. The apparatus according to anyone of embodiments 1 or 58-82, wherein the piezoelectric ultrasonic motor is a rotary piezoelectric ultrasonic motor which operates with at least one of:
a rotational speed in a range of 10 mrad/s-10,000 mrad/s, and
a torque in a range of 20 Nmm-450 Nmm.
84. The apparatus according to anyone of embodiments 1 or 58-83, wherein the piezoelectric ultrasonic motor is a linear piezoelectric ultrasonic motor which operates with at least one of:
a speed in a range of 4 mm/s-100 mm/s,
a stroke length in a range of 4 mm-30 mm, and
a force in a range of 0.5 N-30 N.
85. The apparatus according to anyone of embodiments 1 or 58-84, wherein the at least one piezoelectric motor comprises at least one bimorph piezoelectric actuator.
86. The apparatus according to anyone of embodiments 1 or 58-85, wherein the at least one piezoelectric motor is a reversible piezoelectric motor.
87. The apparatus according to anyone of embodiments 1 or 58-86, wherein the valve is adapted to be placed close to the outlet of the intestine distal of the reservoir out from the body in one of the following positions; close to anus or close to a stoma opening.
88. The apparatus of using a pre-implanted flow control device defined in anyone of embodiments 1 or 58-87, comprising the steps of:
advancing intestinal contents from the reservoir through the patient's intestine in a downstream direction on patient command to defecate, and
opening the valve placed close to anus or stoma before executing the emptying.
89. The apparatus of using a pre-implanted flow control device defined in anyone of embodiments 1 or 58-88, comprising the steps of:
stimulating the intestine in the reservoir thereby advancing intestinal contents from the reservoir through the patient's intestine in a downstream direction on patient command to defecate, and
opening the valve placed close to anus or stoma before executing the emptying.

### Aspect Stoma Restore 4

1. An apparatus for treating a patient, comprising:
a flow control device adapted to permanently reside inside the patient's body and be adapted to control flow of intestinal contents from a reservoir made by patients intestine to empty to outside the patient's body, the flow control device comprising:
an electrical stimulation type pump advancing intestinal contents through the patient's intestine in a downstream direction on patient command, the reservoir created by: a selected suitable portion from the jejunum or ileum, that has been bent to create a 1800 bend where an instrument has made two small holes in the bent area, wherein a stapler instrument with two separate two halves has been introduced through the two small holes in the intestine to clamp the intestines between the two halves of the stapler instrument to be stapled with at least one staple line on each side of the instrument and the two intestinal halves has been cut in between the staple lines to create a larger reservoir, wherein the two small holes in the intestine has been closed with sutures or staplers, and wherein optionally a second intestinal area has been bent to thereby repeat the process at least once as disclosed in this section to increase the size of the reservoir,
a control unit, wherein the control unit is configured to execute at least one of a peristaltic type of emptying of the reservoir and to execute a direct massive blow type of emptying of the reservoir,
at least one valve for opening and closing the intestine to control flow of intestinal contents from the reservoir through the intestine out from the body, the control unit is configured to at least one of:
stimulate the at least first and second electrode when placed on the reservoir folds with maximum 1 second time in between the stimulation of the at least first and second electrodes to be able to execute a stimulation pattern creating a direct massive blow type of emptying of the reservoir, the flow control device further comprising at least one of:
at least two electrodes, at least first and second electrode, adapted to be placed with on the reservoir folds of two stapled halves of intestine laying side by side maximum 2 cm distance from each other in one or more grows of the reservoir and adapted to be covered by suturing or stapling together intestine from both halves of the intestine, wherein the control unit is configured to execute a stimulation pattern creating a direct massive blow type of emptying of the reservoir, wherein the control unit is configured to open the valve when the flow control device is emptying the reservoir.
2. The apparatus according to embodiment 1, wherein the control device comprises:
   a wireless transceiver for communicating wirelessly with an external device,
   a security module, and
a central unit configured to be in communication with the wireless transceiver, the security module and the flow control device:
the wireless transceiver is configured to receive communication from the external device including at least one instruction to the flow control device, and transmit the received communication to the central unit,
the central unit is configured to send secure communication to the security module, derived from the received communication from the external device, and
the security module is configured to at least one of: decrypt at least a portion of the secure communication, and
verify the authenticity of the secure communication, and
the security module is configured to transmit a response communication to the central unit, and
the central unit is configured to communicate the at least one instruction to the flow control device, the at least one instruction being based on:
the response communication, or
a combination of the response communication and the received communication from the external device.
3. The apparatus according to embodiment 1, wherein the security module comprises a set of rules for accepting communication from the central unit.
4. The apparatus according to embodiment 1, wherein the wireless transceiver is configured to be placed in an off-mode, in which no wireless communication can be transmitted or received by the wireless transceiver, and wherein the set of rules comprises a rule stipulating that communication from the central unit is only accepted when the wireless transceiver is placed in the off-mode.
5. The apparatus according to embodiment 1, wherein the set of rules comprises a rule stipulating that communication from the central unit is only accepted when the wireless transceiver has been placed in the off-mode for a specific time period.
6. The apparatus according to embodiment 1, wherein the central unit is configured to verify a digital signature of the received communication from the external device.
7. The apparatus according to embodiment 1, wherein the control device comprises an implantable controller for an implantable medical device, the implantable controller comprises:
   a wireless transceiver for communicating wirelessly with an external device,
   a security module, and a central unit configured to be in communication with the wireless transceiver and the security module.
wherein:
the wireless transceiver is configured to receive data from the external device including at least one instruction to the implantable medical device, and transmit the received communication to the central unit,
the central unit is configured to send the data to the security module, derived from the received communication from the external device, and
the security module is configured to:
decrypt at least a portion of the data or verify the authenticity of the data, and communicate the at least one instruction to the implantable medical device based on a successful decryption or verification of the secure communication.
8. The apparatus for treating a patient according to embodiment 7, wherein the security module comprises a set of rules
9. The apparatus for treating a patient according to embodiment 8, wherein the wireless transceiver is configured to be placed in an off-mode, in which no wireless communication can be transmitted or received by the wireless transceiver, and wherein the set of rules comprises a rule stipulating that data from the central unit is only accepted by the security module when the wireless transceiver is placed in the off-mode.
10. The apparatus for treating a patient according to embodiment 7, wherein the set of rules comprises a rule stipulating that data from the central unit is only accepted when the wireless transceiver has been placed in the off-mode for a specific time period.
11. The apparatus for treating a patient according to embodiment 7, wherein the central unit is configured to verify a digital signature of the received data from the external device and on a positive verification send the received data to the security module.
12. The apparatus according to embodiment 1, wherein the control device comprises an implantable medical device comprising a receiving unit comprising:
at least one coil configured for receiving transcutaneously transferred energy,
a measurement unit configured to measure a parameter related to the energy received by the coil,
a variable impedance electrically connected to the coil,
a switch placed between the variable impedance and the coil for switching off the electrical connection between the variable impedance and the coil, and
a controller configured to:
control the variable impedance for varying the impedance and thereby tune the coil based on the measured parameter, and
control the switch for switching off the electrical connection between the variable impedance and the coil in response to the measured parameter exceeding a threshold value.
13. The apparatus for treating a patient according to embodiment 12, wherein the controller is configured to vary the variable impedance in response to the measured parameter exceeding a threshold value.
14. The apparatus for treating a patient according to embodiment 12, wherein the measurement unit is configured to measure a parameter related to the energy received by the coil over a time period.
15. The apparatus for treating a patient according to embodiment 12, wherein the measurement unit is configured to measure a parameter related to a change in energy received by the coil.
16. The apparatus for f treating a patient according to embodiment 14, wherein the first switch is placed at a first end portion of the coil, and wherein the implantable medical device further comprises a second switch placed at a second end portion of the coil, such that the coil can be completely disconnected from other portions of the implantable medical device.
17. The apparatus according to embodiment 1, wherein the control device comprises an implantable medical device comprising a receiving unit comprising:
at least one coil configured for receiving transcutaneously transferred energy, a measurement unit configured to measure a parameter related to the energy received by the coil,
a first switch is placed at a first end portion of the coil,
a second switch placed at a second end portion of the coil, such that the coil can be completely disconnected from other portions of the implantable medical device, and
a controller configured to control the first and second switch for completely disconnecting the coil from other portions of the implantable medical device on the basis of the measured parameter.
18. The apparatus for treating a patient according to embodiment 17, wherein the controller is configured to control the first and second switch in response to the measured parameter exceeding a threshold value.
19. The apparatus for treating a patient according to embodiment 17, wherein the measurement unit is configured to measure a parameter related to the energy received by the coil over a time period.
20. The apparatus for treating a patient according to embodiment 17, wherein the measurement unit is configured to measure a parameter related to a change in energy received by the coil.
21. The apparatus for treating a patient according to embodiment 17, wherein the receiving unit is configured to receive transcutaneously transferred energy in pulses according to a pulse pattern, and wherein the measurement unit is configured to measure a parameter related to the pulse pattern.
22. The apparatus according to embodiment 1, wherein the control device comprises an implantable medical device configured to receive remote instructions from an external system, the implantable medical device comprising:
   a wireless receiver configured to receive wirelessly transmitted data packets from the external system, a computing unit configured to:
      verify the electronic signature, and use a checksum provided in the data packet to verify the integrity of the instructions.
23. The apparatus for treating a patient according to embodiment 22, wherein the computing unit is configured to decrypt the data packet.
24. The apparatus for treating a patient according to embodiment 22, wherein the computing unit is configured to use the checksum to verify that the bit stream making up the instructions is unchanged.
25. The apparatus for treating a patient according to embodiment 22, wherein the wireless receiver is part of a wireless transceiver.
26. The apparatus for treating a patient according to embodiment 22, wherein the computing unit comprises a memory unit configured to store electronic signatures, and wherein the computing unit is configured to verify the electronic signature my comparing the electronic signature with the electronic signatures stored in the memory unit.
27. The apparatus according to embodiment 1, wherein the control device is configured to receive remote instructions from an external system, wherein control device comprises a list of codes and the external system comprises a list of codes, the apparatus comprising:
   wirelessly receiving encrypted instructions to the flow control device, and decrypting, at the flow control device, the instructions using a code from a position on the list of codes.
28. The apparatus for treating a patient according to embodiment 27, further comprising the steps of:
wirelessly sending position information from the external device to the flow control device, and using the information at the flow control device for selecting the code from the list of codes.
29. The apparatus for treating a patient according to embodiment 27, wherein the step of encrypting, at the external system, the instructions using a code from a position on the list of codes comprises selecting the code on a current position on the list of codes, wherein the apparatus further comprises the step of updating the current position to a new current position after using the code.
30. The apparatus for treating a patient according to embodiment 27, wherein the step of decrypting, at the flow control device, the instructions using a code from a position on the list of codes comprises selecting the code on a current position on the list of codes, wherein the apparatus further comprises the step of updating the current position to a new current position after using the code.
31. The apparatus for treating a patient according to embodiment 29 or 30, wherein the current position comprises a number and wherein the step of updating the current position comprises updating the number to a sequential number.
32. The apparatus according to embodiment 1, wherein the control device comprises an internal communications unit adapted to communicate with an external communications unit using near field magnetic induction.
33. The apparatus for treating a patient according to embodiment 32, wherein: the internal communication unit comprises an internal NFMI receiver and an internal coil connected to the internal NFMI receiver, the internal NFMI receiver being configured to measure an induced voltage in the internal coil,
the external communications unit comprises an external NFMI transmitter and an external coil connected to the external NFMI transmitter, and the external coil and the external NFMI transmitter are configured to modulate a magnetic field for sending data to the flow control device via the internal coil.
34. The apparatus for treating a patient according to embodiment 33, wherein the external NFMI transmitter further comprises a capacitor for tuning the external coil and the external NFMI transmitter.
35. The apparatus for treating a patient according to embodiment 34, wherein the internal NFMI receiver comprises a tunable resistor and capacitor tank for turning the internal coil and the internal NFMI receiver.
36. The apparatus for treating a patient according to embodiment 32, wherein: the internal communication unit comprises an internal NFMI transmitter and an internal coil connected to the internal NFMI transmitter,
the external communications unit comprises an external NFMI receiver and an external coil connected to the external NFMI receiver, the external NFMI receiver being configured to measure an induced voltage in the external coil,
the internal coil and the internal NFMI transmitter are configured to modulate a magnetic field for sending data to the external communications unit via the external coil.
37. The apparatus according to embodiment 1, wherein the control device is adapted to receive transcutaneously and wirelessly transmitted energy, the control device comprising:
a first energy receiving unit, comprising a first coil configured for receiving transcutaneously transferred energy, and a first impedance unit electrically connected to the first coil, the receiving unit being configured to transfer the received energy to flow control device, a second energy receiving unit, comprising a second coil configured for receiving transcutaneously transferred energy and a second impedance unit electrically connected to the second coil, the receiving unit being configured to transfer the received energy to the flow control device,
a measurement unit configured to measure a parameter related to energy transfer, and
a controller configured to control the subcutaneously received energy based on the parameter by controlling the first or the second impedance unit.
38. The apparatus according to embodiment 1, wherein the control device is adapted to receive transcutaneously and wirelessly transmitted energy, the flow control device comprising:
a receiving unit configured for receiving transcutaneously transferred energy and transferring the received energy to the flow control device, comprising:
a first coil portion and a second coil portion, and
a first impedance unit and a second impedance unit, wherein the first impedance unit is connected to the first coil portion and the second impedance unit is connected to the second coil portion,
a measurement unit configured to measure a parameter related to energy transfer, and
a controller configured to control the subcutaneously received energy based on the parameter by controlling the first or the second impedance unit.
39. The apparatus for treating a patient according to embodiment 37 or 38, wherein the first impedance is connected in parallel to the first coil portion and the second impedance is connected in parallel to the second coil portion.
40. The apparatus for treating a patient according to embodiment 37 or 38, wherein one of the first coil portion and the second coil portion are overlapping the other of the first coil portion and the second coil portion, or the first coil portion and the second coil portion are not overlapping with the other of the first coil portion and the second coil portion.
41. The apparatus for treating a patient according to embodiment 37 or 38, wherein the first coil portion and the first impedance unit has a first resonance frequency, and the second coil portion and the second impedance unit has a second resonance frequency.
42. The apparatus according to embodiment 1, wherein the control device comprises:
a first receiving unit comprising:
a first coil configured for receiving transcutaneously transferred energy and transferring the received energy to the flow control device,
a first impedance electrically connected to the coil,
a second receiving unit comprising:
a second coil portion and a third coil portion configured for receiving transcutaneously transferred energy and transferring the received energy to the flow control device, and a second impedance unit and a third impedance unit, wherein the second impedance unit is connected to the second coil portion and the third impedance unit is connected to the third coil portion,
a measurement unit configured to measure a parameter related to energy transfer, and a controller configured to control the subcutaneously received energy based on the parameter by controlling the first, the second or the third impedance unit.
43. The apparatus according to embodiment 1, wherein the control device is configured to recieve wireless energy from an external energy source located outside the patient to an internal energy receiver located inside the patient, the internal energy receiver being connected to an implantable medical device for supplying received energy thereto, the control device being configured to:
determining an accumulated amount of received energy over a time period;
determining a current change in the received energy;
determining a control signal reflecting the accumulated received energy and the change in the transferred or received energy;
controlling the energy transfer based on the control signal.
44. The apparatus for treating a patient according to embodiment 43, wherein determining an accumulated amount of received energy is determined by the internal energy receiver.
45. The apparatus for treating a patient according to embodiment 43, wherein determining a current change is performed by the internal energy receiver.
46. The apparatus for treating a patient according to embodiment 43, wherein the internal energy received comprises a PID regulator for controlling the energy transfer.
47. The apparatus for treating a patient according to embodiment 43, wherein the PID regulator is implemented in a microcontroller.
48. The apparatus for treating a patient according to embodiment 1, wherein the control device is configured to receive voice commands and is configured to:
receive a first audio training phrase, when the flow control device is implanted in the body of the patient,
create a transfer function, the transfer function being based on the first audio training phrase, wherein the transfer function is configured to adjust the amplitude of at least one frequency of audio received at the flow device for enhancing audio received at the control device to facilitate detection of voice commands,
receive a second audio training phrase comprising the voice command, the voice command comprising an instruction for the control of the flow control device,
using the transfer function for generating an enhanced second audio training phrase, and
associating the enhanced second audio training phrase with the instruction for the control of the flow control device.
49. The apparatus for treating a patient according to embodiment 48, wherein adjusting the amplitude comprises at least one of: filtering, cancelling and amplifying the at least one frequency.
50. The apparatus for treating a patient according to embodiment 48, wherein at least one of the first and second audio training phrase is a spoken audio training phrase.
51. The apparatus for treating a patient according to embodiment 50, wherein the spoken audio training phrase is spoken by the patient the implant is implanted in.
52. The apparatus for treating a patient according to embodiment 48, wherein the first audio training phrase comprises the at least one voice command related to an instruction for the control of the flow control device.
53. The apparatus for treating a patient according to embodiment 1, wherein the control device comprises a receiving unit comprising:
at least one coil configured for receiving transcutaneously transferred energy,
a measurement unit configured to measure a parameter related to the energy received by the coil, and
a controller, wherein:
the receiving unit is configured to receive transcutaneously transferred energy in pulses according to a pulse pattern, and
the measurement unit is configured to measure a parameter related to the pulse pattern, and
the controller is configured to control the receiving unit in response to the pulse pattern of the received energy deviating from a predetermined pulse pattern.
54. The apparatus for treating a patient according to embodiment 53, further comprising at least one switch placed in series with the coil for switching of the coil, wherein the controller is configured to control the switch to switch of the coil in response to the pulse pattern of the received energy deviating from a predetermined pulse pattern.
55. The apparatus for treating a patient according to embodiment 53, further comprising a variable impedance electrically connected to the coil, for varying the impedance and thereby tuning the coil, and wherein the controller is configured to control the variable impedance in response to the pulse pattern of the received energy deviating from a predetermined pulse pattern.
56. The apparatus for treating a patient according to embodiment 53, wherein the measurement unit is configured to measure a parameter related to the energy received by the coil over a time period.
57. The apparatus for treating a patient according to embodiment 53, wherein the measurement unit is configured to measure a parameter related to a change in energy received by the coil.
58. The apparatus according to embodiment 1, wherein the control device is configured to empty the reservoir with electrical stimulation simultaneously as the valve is not stimulated and opened.
59. The apparatus according to anyone of embodiments 1 or 58, wherein the valve at least one of a stimulation, hydraulic, pneumatic, mechanical valve or any combination thereof
60. The at least two electrodes according to anyone of embodiments 1 or 58-59, comprising a holding device adapted to be placed on the reservoir folds of two stapled halves of intestine laying side by side holding the electrodes with maximum 2 cm distance away from each other to allow the massive blow type of stimulation pattern and emptying of the reservoir.
61. The apparatus according to anyone of embodiments 1 or 58-60, wherein the control unit is configured to;
train the intestinal wall when the reservoir is not emptied.
62. The apparatus according to anyone of embodiments 1 or 58-61, wherein the control unit is configured to;
stimulate the muscle or neural tissue of the intestine in a peristaltic way in the opposite direction of intestinal flow by the at least two electrodes.
63. The apparatus according to anyone of embodiments 1 or 58-62, wherein the control unit is configured to;
training the intestinal muscle wall to an extent, that together with the valve that may be slightly released, constricting the intestine to hinder intestinal contents flowing through the patient's intestine in a downstream direction during the training execution.
64. The apparatus according to anyone of embodiments 1 or 58-63, comprising at least one sensor configured to sense at least one of a physical parameter of the patient's body and a functional parameter of the apparatus.
65. The apparatus according to anyone of embodiments 1 or 58-64, comprising at least one of the following;
wherein the at least one sensor is configured to sense the physical parameter in the patient's body, comprising sensing at least one of the following physical parameters of the patient: a pressure measured on the outside of the reservoir, a pressure within the patient's intestine, an expansion of the reservoir, and a distension of an intestinal wall of the patient's intestine. wherein the at least one sensor is configured to sense the functional parameter of the apparatus when implanted, comprising sensing at least one of the following parameters: a pressure against a part of the system such as the valve, an electrical parameter such as voltage, current or energy balance, a position or movement of a movable part of the system, and any stimulation parameter in relation to the system.
66. The apparatus according to anyone of embodiments 1 or 58-65, configured to provide a signal when a value for the physical or functional parameter sensed is beyond a predetermined threshold value.
67. The apparatus according to anyone of embodiments 1 or 58-66, comprising a vibration generation unit for stimulating at least one of the intestine forming the reservoir and the intestine closed by the valve.
68. The apparatus according to anyone of embodiments 1 or 58-67, wherein the vibration generating unit is attached to the casing, so that vibrations generated by the vibration generating unit can travel to the casing.
69. The apparatus according to anyone of embodiments 1 or 58-68, wherein the implantable vibration device (110) comprises an internal controller (CI).
70. The apparatus according to anyone of embodiments 1 or 58-69, wherein a internal controller (CI) is configured to wirelessly receive vibration control data for controlling the vibration of the implantable vibration device.
71. The apparatus according to anyone of embodiments 1 or 58-70, wherein the apparatus does not comprise any metallic parts and does not comprise any magnetic parts.
72. The apparatus according to anyone of embodiments 1 or 58-71, wherein the vibration generating unit is operated by a piezoelectric motor.
73. The apparatus according to anyone of embodiments 1 or 58-72, wherein at least one piezoelectric motor is arranged for driving a pump for pumping fluid to at least one of the valve or for supporting the emptying of the reservoir.
74. The apparatus according to anyone of embodiments 1 or 58-73, wherein the at least one piezoelectric motor comprises a piezoelectric inchworm motor.
75. The apparatus according to anyone of embodiments 1 or 58-74, wherein the at least one piezoelectric motor comprises a piezoelectric inertial motor.
76. The apparatus according to anyone of embodiments 1 or 58-75, wherein the at least one piezoelectric motor comprises a piezoelectric walk-drive motor.
77. The apparatus according to anyone of embodiments 1 or 58-76, wherein the piezoelectric motor is a linear piezoelectric motor.
78. The apparatus according to anyone of embodiments 1 or 58-77, wherein the linear piezoelectric motor operates with at least one of:
a speed in a range of 1 mm/s to 10 mm/s,
a stroke length in a range of 4 mm-30 mm, and
a force in a range of 2 N-30 N.
79. The apparatus according to anyone of embodiments 1 or 58-78, wherein the piezoelectric motor is a rotary piezoelectric motor.
80. The apparatus according to anyone of embodiments 1 or 58-79, wherein the rotary piezoelectric motor which operates with at least one of:
   a rotational speed in a range of 1 mrad/s-100 mrad/s, and
   a torque in a range of 100 Nmm-900 Nmm.
81. The apparatus according to anyone of embodiments 1 or 58-80, wherein the at least one piezoelectric motor comprises a piezoelectric ultrasonic motor.
82. The apparatus according to anyone of embodiments 1 or 58-81, wherein the piezoelectric ultrasonic motor is a traveling wave ultrasonic motor or a standing wave ultrasonic motor.
83. The apparatus according to anyone of embodiments 1 or 58-82, wherein the piezoelectric ultrasonic motor is a rotary piezoelectric ultrasonic motor which operates with at least one of:
   a rotational speed in a range of 10 mrad/s-10,000 mrad/s, and
   a torque in a range of 20 Nmm-450 Nmm.
84. The apparatus according to anyone of embodiments 1 or 58-83, wherein the piezoelectric ultrasonic motor is a linear piezoelectric ultrasonic motor which operates with at least one of:
   a speed in a range of 4 mm/s-100 mm/s,
   a stroke length in a range of 4 mm-30 mm, and
   a force in a range of 0.5 N-30 N.
85. The apparatus according to anyone of embodiments 1 or 58-84, wherein the at least one piezoelectric motor comprises at least one bimorph piezoelectric actuator.
86. The apparatus according to anyone of embodiments 1 or 58-85, wherein the at least one piezoelectric motor is a reversible piezoelectric motor.
87. The apparatus according to anyone of embodiments 1 or 58-86, wherein the valve is adapted to be placed close to the outlet of the intestine distal of the reservoir out from the body in one of the following positions; close to anus or close to a stoma opening.
88. The apparatus of using a pre-implanted flow control device defined in anyone of embodiments 1 or 58-87, comprising the steps of:
   advancing intestinal contents from the reservoir through the patient's intestine in a downstream direction on patient command to defecate, and
   opening the valve placed close to anus or stoma before executing the emptying.
89. The apparatus of using a pre-implanted flow control device defined in anyone of embodiments 1 or 58-88, comprising the steps of:
   stimulating the intestine in the reservoir thereby advancing intestinal contents from the reservoir through the patient's intestine in a downstream direction on patient command to defecate, and
   opening the valve placed close to anus or stoma before executing the emptying.

1. An external device configured for communication with an implantable medical device when implanted in a patient, the external device comprising:
at least one first wireless transceiver configured for communication with the implantable medical device using a first network protocol, for determining a distance between the external device and the implantable medical device, and
at least one second wireless transceiver configured for communication with the implantable medical device using a second network protocol, for transferring data between the external device and the implantable medical device.

2. The external device according to claim 1, wherein the first wireless transceiver comprises an UWB transceiver.

3. The external device according to claim 1, wherein the first wireless transceiver is configured for transcutaneous energy transfer for at least one of:
powering an energy consuming component of the implantable medical device, and
charging an implantable energy storage unit.

4. The external device according to claim 1, wherein the second network protocol is a standard network protocol.

5. The external device according to claim 1, wherein the second wireless transceiver comprises a Bluetooth transceiver.

6. The external device according to claim 1, wherein the external device is further configured to communicate with a second external device using said at least one wireless transceiver.

7. The external device according to claim 1, wherein the external device is configured for determining a distance between the external device and the implantable medical device by determining the RSSI.

8. (canceled)

9. The external device according claim 1, wherein a communication range of the first network protocol is less than a communication range of the second network protocol.

10. The external device according to claim 1, wherein a frequency band of the first network protocol differs from a frequency band of the second network protocol.

11. The external device according to claim 1, wherein the external device is configured to authenticate the implantable medical device if the determined distance between the external device and the implantable medical device is less than a predetermined threshold value.

12. The external device according to claim 11, wherein the external device is configured to allow the transfer of data between the external device and the implantable medical device after the implantable medical device has been authenticated.

14. An implantable medical device configured for communication with an external device, the implantable medical device comprising:
at least one first wireless transceiver configured for communication with the external device using a first network protocol, for determining a distance between the external device and the implantable medical device, and
at least one second wireless transceiver configured for communication with the external device using a second network protocol, for transferring data between the external device and the implantable medical device.

15. The implantable medical device according to claim 14, wherein the first wireless transceiver comprises an UWB transceiver, and/or wherein the second wireless transceiver comprises a Bluetooth transceiver.

16. The implantable medical device according to claim 13, wherein the first wireless transceiver is configured for transcutaneous energy transfer for at least one of:
powering an energy consuming component of the implantable medical device, and
charging an implantable energy storage unit.

17. (canceled)

18. (canceled)

19. The implantable medical device according to claim 12, wherein the implantable medical device is further configured to communicate with a second external device using said at least one wireless transceiver.

20. The implantable medical device according to claim 12, wherein the implantable medical device is configured for determining a distance between the external device and the implantable medical device by determining the RSSI.

21. (canceled)

22. The implantable medical device according to claim 12, wherein a communication range of the first network protocol is less than a communication range of the second network protocol.

23. The implantable medical device according to claim 12, wherein a frequency band of the first network protocol differs from a frequency band of the second network protocol.

24. The implantable medical device according to claim 12, wherein the implantable medical device is configured to authenticate the external device if the determined distance between the external device and the implantable medical device is less than a predetermined threshold value.

25. The implantable medical device according to claim 24, wherein the implantable medical device is configured to allow the transfer of data between the implantable medical device and the external device after the external device has been authenticated.

26. (canceled)
